(19) United States
(12) Patent Application Publication
(10) Pub. No.: US 2020/0377513 A1
(43) Pub. Date: Dec. 3, 2020

(54) NOVEL COMPOUND AND ORGANIC ELECTROLUMINESCENCE DEVICE

(71) Applicant: IDEMITSU KOSAN CO.,LTD., Tokyo (JP)

(72) Inventors: Keita SEDA, Sodegaura-shi (JP); Ryota TAKAHASHI, Sodegaura-shi (JP); Yuki NAKANO, Sodegaura-shi (JP); Yuichiro KAWAMURA, Sodegaura-shi (JP); Hidetsugu IKEDA, Sodegaura-shi (JP)

(73) Assignee: IDEMITSU KOSAN CO.,LTD., Tokyo (JP)

(21) Appl. No.: 16/759,803

(22) PCT Filed: Oct. 31, 2018
(86) PCT No.: PCT/JP2018/040575

§ 371 Date: Apr. 28, 2020

(30) Foreign Application Priority Data
Nov. 2, 2017 (JP) 2017-213234

(51) Int. Cl. C07D 493/04; H01L 51/00

(52) U.S. Cl. CPC C07D 493/04; H01L 51/0072; H01L 51/006; H01L 51/0073

(57) ABSTRACT

A compound represented by the following formula (1):

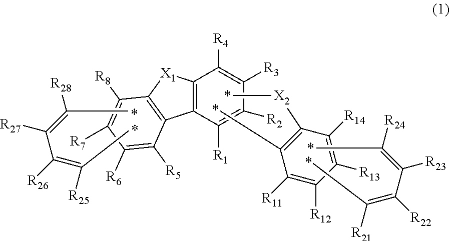

### TECHNICAL FIELD

The invention relates to a novel compound and an organic electroluminescence device using the same.

### BACKGROUND ART

When voltage is applied to an organic electroluminescence device (hereinafter, referred to as an organic EL device in several cases), holes and electrons are injected into an emitting layer from an anode and a cathode, respectively. Then, thus injected holes and electrons are recombined in the emitting layer, and excitons are formed therein.

The organic EL device includes the emitting layer between the anode and the cathode. Further, the organic EL device has a stacked structure including an organic layer such as a hole-injecting layer, a hole-transporting layer, an electron-injecting layer, and an electron-transporting layer in several cases.

Patent Document 1 to 3 discloses a compound used as a material for an organic electroluminescence device.

### RELATED ART DOCUMENTS

### Patent Documents

[Patent Document 1] WO2006122630A1
[Patent Document 2] JP 2010-045281 A
[Patent Document 3] JP 2012-028548 A

### SUMMARY OF THE INVENTION

It is an object of the present invention to provide a novel compound which is high in fluorescent quantum yield and can be used as a material for an organic electroluminescence device, as well as an organic electroluminescence device using the same.

According to an aspect of the present invention, a compound represented by the following formula (1) is provided.

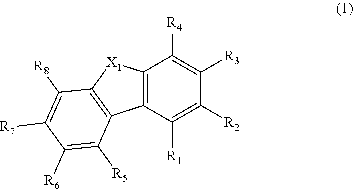

wherein in the formula (1),

one pair among R₁ and R₂, R₂ and R₃, and R₃ and R₄ is respectively bonded with a divalent group represented by the following formula (11);

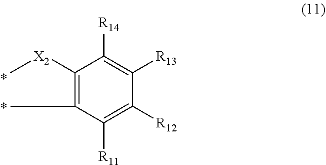

wherein X₁ and X₂ are independently O, S or C(R′)₂;

at least one pair among R₁₁ and R₁₂, R₁₂ and R₁₃, and R₁₃ and R₁₄ is respectively bonded with a divalent group represented by the following formula (12a);

at least one pair among R₅ and R₆, R₆ and R₇, and R₇ and R₈ is respectively bonded with a divalent group represented by the following formula (12b);

when a plurality of divalent groups represented by the following formulas (12a) and (12b) are present, the plurality of divalent groups represented by the following formulas (12a) and (12b) may be the same or different;

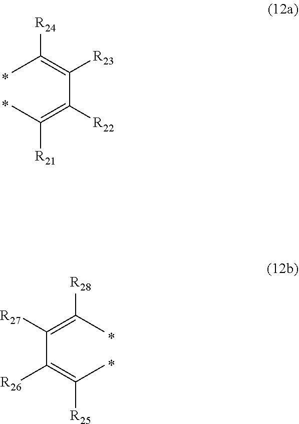

R′, R₂₁ to R₂₈, R₁ to R₄ which are not bonded with a divalent group represented by the formula (11), R₁₁ to R₁₄ which are not bonded with a divalent group represented by the formula (12a), and R₅ to R₈ which are not bonded with a divalent group represented by the formula (12b) independently represent a hydrogen atom, a substituted or unsubstituted alkyl group including 1 to 50 carbon atoms, a substituted or unsubstituted haloalkyl group including 1 to 50 carbon atoms, a substituted or unsubstituted alkenyl group including 2 to 50 carbon atoms, a substituted or unsubstituted alkynyl group including 2 to 50 carbon atoms, a substituted or unsubstituted cycloalkyl group including 3 to 50 carbon atoms that form a ring (hereinafter referred to as “ring carbon atoms”), a substituted or unsubstituted alkoxy group including 1 to 50 carbon atoms, a substituted or unsubstituted alkylthio group including 1 to 50 carbon atoms, a substituted or unsubstituted aryloxy group including 6 to 50 ring carbon atoms, a substituted or unsubstituted arylthio group including 6 to 50 ring carbon atoms, a substituted or unsubstituted aralkyl group including 7 to 50 carbon atoms, —Si(R₃₁)(R₃₂)(R₃₃), —C(═O)R₃₄, —COOR₃₅, a halogen atom, a cyano group, a nitro group, a substituted or unsubstituted aryl group including 6 to 50 ring carbon atoms, a substituted or unsubstituted monovalent heterocyclic group including 5 to 50 atoms that form a ring (hereinafter referred to as “ring atoms”), or a group represented by the following formula (13);

provided that at least one of R₂₁ to R₂₈, R₁ to R₄ which are not bonded with a divalent group represented by the formula (11), R₁ to R₁₄ which are not bonded with a divalent group represented by the formula (12a), and R₅ to R₈ which are not bonded with a divalent group represented by the formula (12b) is a group represented by the following formula (13);

two R's may be the same or different;

R₃₁ to R₃₅ are independently a hydrogen atom, a substituted or unsubstituted alkyl group including 1 to 50 carbon atoms, a substituted or unsubstituted aryl group including 6 to 50 ring carbon atoms, or a substituted or unsubstituted monovalent heterocyclic group including 5 to 50 ring atoms;

when a plurality of R₃₁ to R₃₅ are present, the plurality of R₃₁ to R₃₅ may be independently the same or different;

when a plurality of groups represented by the following formula (13) are present, the plurality of groups represented by the following formula (13) may be the same or different;

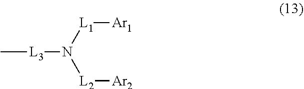

wherein in the formula (13),

L₁ to L₃ are independently a single bond, a substituted or unsubstituted alkylene group including 1 to 30 carbon atoms, a substituted or unsubstituted arylene group including 6 to 30 ring carbon atoms, or a substituted or unsubstituted divalent heterocyclic group including 5 to 30 ring atoms;

Ar₁ and Ar₂ are independently a substituted or unsubstituted alkyl group including 1 to 50 carbon atoms, a substituted or unsubstituted aryl group including 6 to 50 ring carbon atoms or a substituted or unsubstituted monovalent heterocyclic group including 5 to 50 ring atoms, and Ar₁ and Ar₂ may be bonded with each other via a single bond or via —O—,—S— or —C(R)₂—; and

R is a substituent, and two R's may be the same or different.

According to an aspect of the present invention, a material for an organic electroluminescence device containing a compound represented by the formula (1) is provided.

According to an aspect of the present invention, an organic electroluminescence device containing a cathode, an anode, and at least one organic layer disposed between the cathode and the anode, wherein at least one of the at least one organic layer contains the compound represented by the formula (1), is provided.

According to an aspect of the present invention, an electronic appliance provided with the organic electroluminescence device is provided.

According to the present invention, a novel compound which is high in fluorescent quantum yield and can be used as a material for an organic electroluminescence device can be provided.

### BRIEF DESCRIPTION OF THE DRAWINGS

FIG. 1 is a diagram showing a schematic configuration of an embodiment of an organic EL device according to an aspect of the present invention.

FIG. 2 is a diagram showing a schematic configuration of another embodiment of an organic EL device according to an aspect of the present invention.

### DETAILED DESCRIPTION OF THE INVENTION

In the present specification, a hydrogen atom means an atom including isotopes different in the number of neutrons, namely, a protium, a deuterium and a tritium.

In the present specification, the number of “ring carbon atoms” represents the number of carbon atoms among the atoms which forms a subject ring itself of a compound having a structure in which atoms are bonded in a ring form (for example, a monocyclic compound, a fused ring compound, a cross-linked compound, a carbocyclic compound or a heterocyclic compound). When the subject ring is substituted by a substituent, the carbon contained in the substituent is not included in the number of ring carbon atoms. The same shall apply to the number of “ring carbon atoms” described below, unless otherwise noted. For example, a benzene ring includes 6 ring carbon atoms, a naphthalene ring includes 10 ring carbon atoms, a pyridinyl group includes 5 ring carbon atoms, and a furanyl group includes 4 ring carbon atoms. Further, when the benzene ring or the naphthalene ring is substituted by, for example, an alkyl group as a substituent, the number of carbon atoms of the alkyl group is not included in the number of ring carbon atoms. When a fluorene ring is bonded with, for example, a fluorene ring as a substituent (including a spirofluorene ring), the number of carbon atoms of the fluorene ring as a substituent is not included in the number of ring carbon atoms.

In the present specification, the number of “ring atoms” represents the number of atoms forming a subject ring itself of a compound having a structure in which atoms are bonded in a ring form (for example, a monocycle, a fused ring and a ring assembly) (for example, a monocyclic compound, a fused ring compound, a cross-linked compound, a carbocyclic compound or a heterocyclic compound). The atoms that do not form the ring (e.g., a hydrogen atom that terminates the bond of the atoms that form the ring) or the atoms contained in a substituent where the ring is substituted by the substituent is not included in the number of ring atom. The same shall apply to the number of “ring atoms” described below, unless otherwise noted. For example, a pyridine ring includes 6 ring atoms, a quinazoline ring includes 10 ring atoms, and a furan ring includes 5 ring atoms. A hydrogen atom independently bonded with a carbon atom of the pyridine ring or the quinazoline ring or an atom forming the substituent is not included in the number of ring atoms. When a fluorene ring is bonded with, for example, a fluorene ring as a substituent (including a spirofluorene ring), the number of atoms of the fluorene ring as a substituent is not included in the number of ring atoms.

In the present specification, “including XX to YY carbon atoms” in the expression “a substituted or unsubstituted ZZ group including XX to YY carbon atoms” represents the number of carbon atoms when the ZZ group is unsubstituted, and does not include the number of carbon atoms of the substituent when the ZZ group is substituted. Here, “YY” is larger than “XX,” and “XX” and “YY” independently mean an integer of 1 or more.

In the present specification, “including XX to YY atoms” in the expression “a substituted or unsubstituted ZZ group including XX to YY atoms” represents the number of atoms when the ZZ group is unsubstituted, and does not include the number of atoms of the substituent when the ZZ group is substituted. Here, “YY” is larger than “XX,” and “XX” and “YY” independently mean an integer of 1 or more.

The term “unsubstituted” in the context of “a substituted or unsubstituted” means that the substituent is not bonded and a hydrogen atom is bonded.

Specific examples of each substituent in the present specification include the following.

Examples of the unsubstituted alkyl group including 1 to 50 carbon atoms (preferably 1 to 30, more preferably 1 to 18, and still more preferably 1 to 5) include, for example, a methyl group, an ethyl group, a propyl group, an isopropyl group, an n-butyl group, an s-butyl group, an isobutyl group, a t-butyl group, an n-pentyl group, an n-hexyl group, an n-heptyl group, an n-octyl group, and the like.

Examples of the substituted alkyl group including 1 to 50 carbon atoms (preferably 1 to 30, more preferably 1 to 18, and still more preferably 1 to 5) include a hydroxymethyl group, a 1-hydroxyethyl group, a 2-hydroxyethyl group, a 2-hydroxyisobutyl group, a 1,2-dihydroxyethyl group, a 1,3-dihydroxyisopropyl group, a 2,3-dihydroxy-t-butyl group, a 1,2,3-trihydroxypropyl group, a chloromethyl group, a 1-chloroethyl group, a 2-chloroethyl group, a 2-chloroisobutyl group, a 1,2-dichloroethyl group, a 1,3-dichloroisopropyl group, a 2,3-dichlorot-butyl group, a 1,2,3-trichloropropyl group, a bromomethyl group, a 1-bromoethyl group, a 2-bromoethyl group, a 2-bromoisobutyl group, a 1,2-dibromoethyl group, a 1,3-dibromoisopropyl group, a 2,3-dibromo-t-butyl group, a 1,2,3-tribromopropyl group, an iodomethyl group, a 1-iodoethyl group, a 2-iodoethyl group, a 2-iodoisobutyl group, a 1,2-diiodoethyl group, a 1,3-diiodoisopropyl group, a 2,3-diiodo-t-butyl group, a 1,2,3-triiodopropyl group, a cyanomethyl group, a 1-cyanoethyl group, a 2-cyanoethyl group, a 2-cyanoisobutyl group, a 1,2-dicyanoethyl group, a 1,3-dicyanoisopropyl group, a 2,3-dicyano-t-butyl group, a 1,2,3-tricyanopropyl group, a nitromethyl group, a 1-nitroethyl group, a 2-nitroethyl group, a 2-nitroisobutyl group, a 1,2-dinitroethyl group, a 1,3-dinitroisopropyl group, a 2,3-dinitro-t-butyl group, a 1,2,3-trinitropropyl group, a 1-pyrrolylmethyl group, a 2-(1-pyrrolylethyl group, a 1-hydroxy-2-phenylisopropyl group, a 1-chloro-2-phenylisopropyl group, and the like.

The substituted or unsubstituted haloalkyl group including 1 to 50 carbon atoms is a group in which one or more of hydrogen atoms of the alkyl group is substituted by a halogen atom. As the substituted or unsubstituted haloalkyl group including 1 to 50 carbon atoms, a group obtained by substituting one or more halogen atoms in the above-mentioned substituted or unsubstituted alkyl group including 1 to 50 carbon atoms can be given.

As the unsubstituted alkenyl group including 2 to 50 (preferably 2 to 30, more preferably 2 to 18) carbon atoms, a vinyl group, an allyl group, a 1-butenyl group, a 2-butenyl group, a 3-butenyl group, a 1,3-butanedienyl group, a 1-methylvinyl group, a 1-methylallyl group, a 1,1-dimethylallyl group, a 2-methylallygroup, a 1,2-dimethylallyl group or the like can be given.

As the unsubstituted alkynyl group including 2 to 50 (preferably 2 to 30, more preferably 2 to 18) carbon atoms, an ethynyl group or the like can be given.

As the unsubstituted cycloalkyl group including 3 to 50 (preferably 3 to 30, more preferably 3 to 18, and further preferably 3 to 6) ring carbon atoms, a cyclopropyl group, a cyclobutyl group, a cyclopentyl group, a cyclohexyl group, a 4-methylcyclohexyl group, a 1-adamantyl group, a 2-adamantyl group, a 1-norbomyl group, a 2-norbomyl group or the like can be given.

The unsubstituted alkoxy group including 1 to 50 (preferably 1 to 30, more preferably 1 to 18) carbon atoms is represented by —OX. As examples of X, the alkyl group including 1 to 50 carbon atoms mentioned above can be given, for example.

The unsubstituted alkylthio group including 1 to 50 (preferably 1 to 30, more preferably 1 to 18) carbon atoms is represented by —SX. As examples of X, the alkyl group including 1 to 50 carbon atoms mentioned above can be given, for example.

As the unsubstituted aryl group including 6 to 50 (preferably 6 to 30, more preferably 6 to 18) ring carbon atoms, a phenyl group, a 1-naphthyl group, a 2-naphthyl group, a 1-anthryl group, a 2-anthryl group, a 9-anthryl group, a 9-phenanthryl group, a 1-phenanthryl group, a 2-phenanthryl group, a 3-phenanthryl group, a 4-phenanthryl group, a 1-naphthacenyl group, a 2-naphthacenyl group, a 9-naphthacenyl group, a 1-pyrenyl group, a 2-pyrenyl group, a 4-pyrenyl group, a 2-biphenylyl group, a 3-biphenylyl group, a 4-biphenylyl group, a p-terphenyl-4-yl group, a p-terphenyl-3-yl group, a p-terphenyl-2-yl group, a m-terphenyl-4-yl group, a m-terphenyl-3-yl group, a m-terphenyl-2-yl group, a fluorenyl group or the like can be given.

Among these, a phenyl group, a naphthyl group, a biphenylyl group, a terphenyl group, a pyrenyl group, a phenanthryl group and a fluorenyl group are preferable. A phenyl group, a naphthyl group, a biphenylyl group, a terphenyl group, a pyrenyl group and a fluorenyl group are more preferable.

As the substituted aryl group including 6 to 50 (preferably 6 to 30, more preferably 6 to 18) ring carbon atoms, an o-tolyl group, a m-tolyl group, a p-tolyl group, a para-isopropylphenyl group, a meta-isopropylphenyl group, an ortho-isopropylphenyl group, a p-t-butylphenyl group, a meta-t-butylphenyl group, an ortho-t-butylphenyl group, a 3,4,5-trimethylphenyl group, a 4-phenoxyphenyl group, a 4-methoxyphenyl group, a 3,4-dimethoxyphenyl group, a 3,4,5-trimethoxyphenyl group, a 4-(phenylsulfanylphenyl group, a 4-(methylsulfanylphenyl group, a N′,N′-dimethyl-N-phenyl group, a N′,N′-dimethyl-N-phenyl group, a 2,6-dimethylphenyl group, a (2-phenylpropylphenyl group, a 3-methyl-2-naphthyl group, a 4-methyl-1-naphthyl group, a 4-methyl-1-anthryl group, a 4′-methylbiphenylyl group, a 4″-t-butyl-p-terphenyl-4-yl group, a 9,9-dimethylfluorenyl group, a 9,9-diphenylfluorenyl group, a 9,9′-spirobifluorenyl group, a 9,9-di(4-methylphenylfluorenyl group, a 9,9-di(4-isopropylphenylfluorenyl group, a 9,9-di(4-t-butylphenylfluorenyl group, a chrysenyl group, a fluoranthenyl group or the like can be given.

As the unsubstituted arylene group including 6 to 50 (preferably 6 to 30, more preferably 6 to 18) ring carbon atoms, a divalent group formed of an aromatic hydrocarbon ring constituting the aryl group including 6 to 50 ring carbon atoms exemplified above can be given.

The unsubstituted aryloxy group including 6 to 50 (preferably 6 to 30, more preferably 6 to 18) ring carbon atoms is represented by —OY As examples of Y the aryl group including 6 to 50 ring carbon atoms mentioned above can be given.

The unsubstituted arylthio group including 6 to 50 (preferably 6 to 30, more preferably 6 to 18) ring carbon atoms is represented by —SY As examples of Y the aryl group including 6 to 50 ring carbon atoms mentioned above can be given.

As the unsubstituted aralkyl group including 7 to 50 (preferably 7 to 30, more preferably 7 to 18) carbon atoms, a benzyl group, a 1-phenylethyl group, a 2-phenylethyl group, a 1-phenylisopropyl group, a 2-phenylisopropyl group, a phenyl-t-butyl group, a α-naphthylmethyl group, a 1-α-naphthylethyl group, a 2-α-naphthylethyl group, 1-α-naphthylisopropyl group, a 2-α-naphthylisopropyl group, a β-naphthylmethyl group, a 1-β-naphthylethyl group, a 2-pi-naphthylethyl group, a 1-β-naphthylisopropyl group, a 2-β-naphthylisopropyl group or the like can be given.

As the substituted aralkyl group including 7 to 50 (preferably 7 to 30, more preferably 7 to 18) carbon atoms, a p-methylbenzyl group, a m-methylbenzyl group, an o-methylbenzyl group, a p-chlorobenzyl group, a m-chlorobenzyl group, an o-chlorobenzyl group, a p-bromobenzyl group, a m-bromobenzyl group, an o-bromobenzyl group, a p-iodobenzyl group, a m-iodobenzyl group, an o-iodobenzyl group, a p-hydroxybenzyl group, a m-hydroxybenzyl group, an o-hydroxybenzyl group, a p-nitrobenzyl group, a m-nitrobenzyl group, an o-nitrobenzyl group, a p-cyanobenzyl group, a m-cyanobenzyl group, an o-cyanobenzyl group or the like can be given, for example.

As the unsubstituted monovalent heterocyclic group including 5 to 50 (preferably 5 to 30, more preferably 5 to 18) ring atoms, a pyrrolyl group, a pyrazinyl group, a pyridinyl group, an indolyl group, an isoindolyl group, a furyl group, a benzofuranyl group, an isobenzofuranyl group, a dibenzofuranyl group, a dibenzothiophenyl group, a quinolyl group, an isoquinolyl group, a quinoxalinyl group, a carbazolyl group, a phenanthridinyl group, an acridinyl group, a phenanthrolinyl group, and a thienyl group orthe like, and a monovalent group formed of a pyridine ring, a pyrazine ring, a pyrimidine ring, a pyridazine ring, a triazine ring, an indole ring, a quinoline ring, an acridine ring, a pyrrolidine ring, a dioxane ring, a piperidine ring, a morpholine ring, a piperazine ring, a carbazole ring, a furan ring, a thiophene ring, an oxazole ring, an oxadiazole ring, a benzoxazole ring, a thiazole ring, a thiadiazole ring, a benzothiazole ring, a triazole ring, an imidazole ring, a benzimidazole ring, a pyran ring, a dibenzofuran ring, a benzo[a]dibenzofuran ring, a benzo[b]dibenzofuran ring and benzo[c]dibenzofuran ring, a 1,3-benzodioxole ring, a 2,3-dihydro-1,4-benzodioxine ring, a phenanthro[4,5-bcd] furan ring, a benzophenoxazine ring or the like can be given.

As the hetero atom constituting the heterocyclic group, in addition to a typical hetero atom such as S, O, N or the like, Si, Ge, Se or the like can be given.

As the unsubstituted divalent heterocyclic group including 5 to 50 (preferably 5 to 30, more preferably 5 to 18) ring carbon atoms, a divalent group formed of the -exemplified groups and the monovalent heterocyclic group or the like can be given.

As the substituted or unsubstituted monovalent heterocyclic group including 5 to 50 ring atoms, the following groups are included. As the divalent heterocyclic group including 5 to 50 ring atoms, groups obtained by forming the following groups into divalent groups are also included.

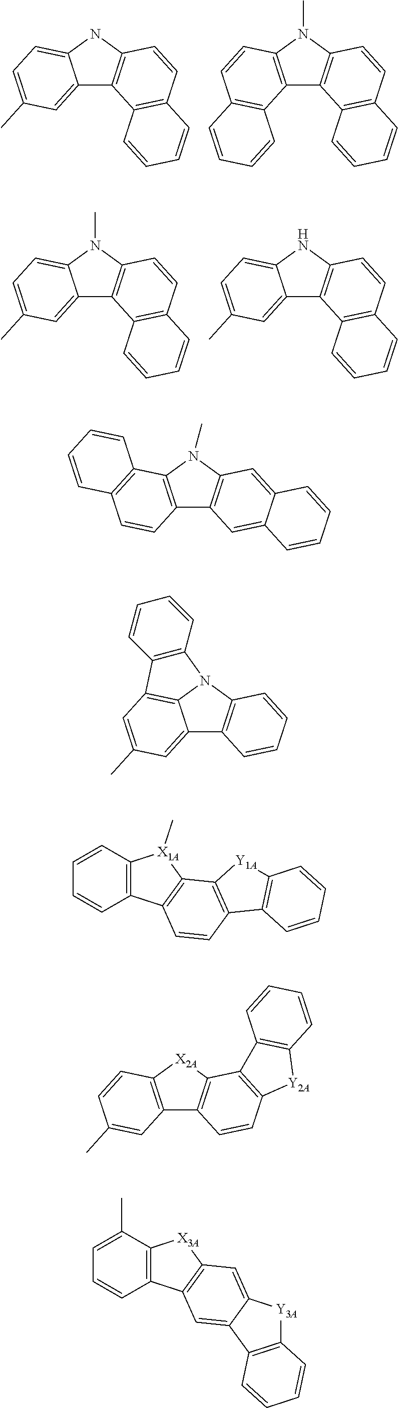

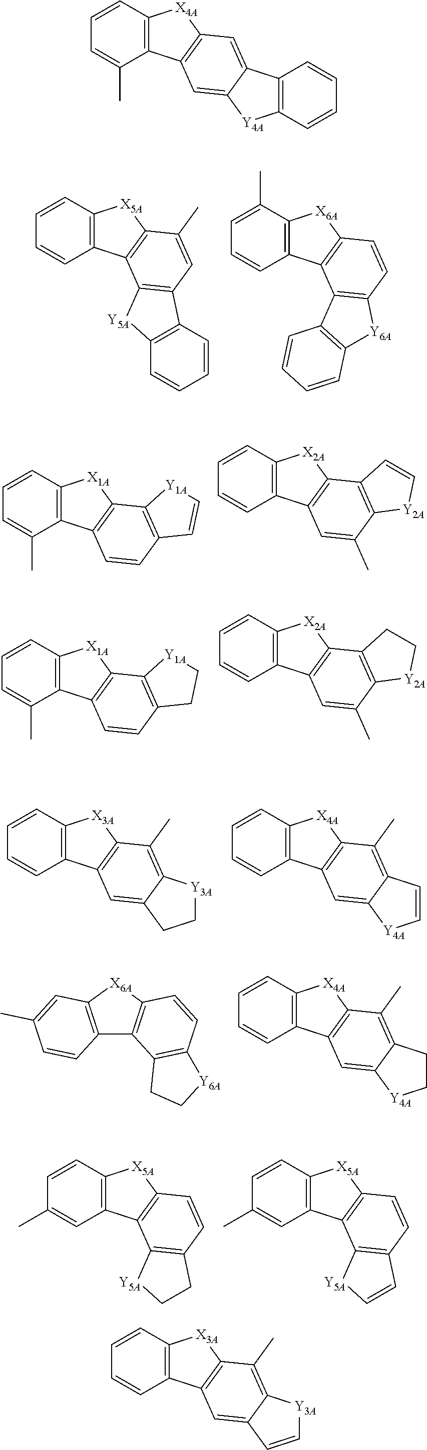

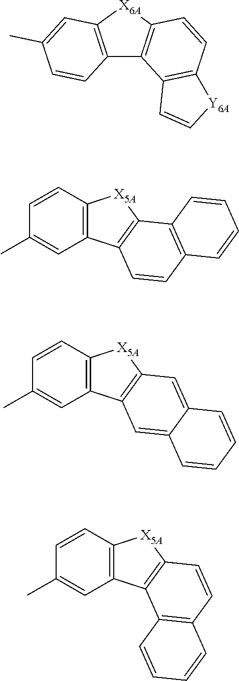

wherein X_{1A} to X_{6A} and Y_{1A} to Y_{6A} are independently an oxygen atom, a sulfur atom, a —NZ— group or a —NH— group. Z is a substituted or unsubstituted aryl group including 6 to 50 ring carbon atoms, a substituted or unsubstituted monovalent heterocyclic group including 5 to 50 ring atoms or a substituted or unsubstituted alkyl group including 1 to 50 carbon atoms. When two or more Zs are present, the two or more Zs may be the same or different.

As the halogen atom, a fluorine atom, a chlorine atom, a bromine atom, an iodine atom or the like can be given.

The new compound according to an aspect of the invention is represented by the following formula (1):

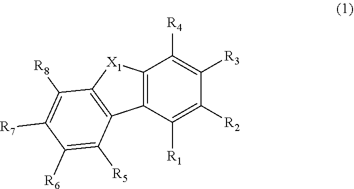

wherein in the formula (1),

one pair of R₁ and R₂, R₂ and R₃, and R₃ and R₄ is respectively bonded with a divalent group represented by the formula (11):

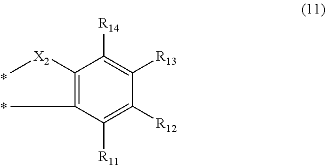

wherein X₁ and X₂ are independently O, S or C(R′)₂;

at least one pair among R₁₁ and R₁₂, R₁₂ and R₁₃, as well as R₁₃ and R₁₄ is respectively bonded with a divalent group represented by the following formula (12a);

at least one pair among R₅ and R₆, R₆ and R₇, as well as R₇ and R₈ is respectively bonded with a divalent group represented by the following formula (12b);

when a plurality of divalent groups represented by the following formulas (12a) and (12b) are present, the plurality of divalent groups represented by the following formulas (12a) and (12b) may be the same or different;

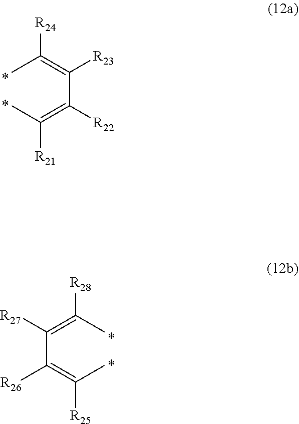

R′, R₂₁ to R₂₈, R₁ to R₄ which are not bonded with a divalent group represented by the formula (11), R₁₁ to R₁₄ which are not bonded with a divalent group represented by the formula (12a), and R₅ to R₈ which are not bonded with a divalent group represented by the formula (12b) independently represent a hydrogen atom, a substituted or unsubstituted alkyl group including 1 to 50 carbon atoms, a substituted or unsubstituted haloalkyl group including 1 to 50 carbon atoms, a substituted or unsubstituted alkenyl group including 2 to 50 carbon atoms, a substituted or unsubstituted alkynyl group including 2 to 50 carbon atoms, a substituted or unsubstituted cycloalkyl group including 3 to 50 ring carbon atoms, a substituted or unsubstituted alkoxy group including 1 to 50 carbon atoms, a substituted or unsubstituted alkylthio group including 1 to 50 carbon atoms, a substituted or unsubstituted aryloxy group including 6 to 50 ring carbon atoms, a substituted or unsubstituted arylthio group including 6 to 50 ring carbon atoms, a substituted or unsubstituted aralkyl group including 7 to 50 carbon atoms, —Si(R₃₁)(R₃₂)(R₃₃), —C(═O)R₃₄, —COOR₃₅, a halogen atom, a cyano group, a nitro group, a substituted or unsubstituted aryl group including 6 to 50 ring carbon atoms, a substituted or unsubstituted monovalent heterocyclic group including 5 to 50 ring atoms, or a group represented by the following formula (13);

provided that at least one of R₂₁ to R₂₈, R₁ to R₄ which are not bonded with a divalent group represented by the formula (11), R₁₁ to R₁₄ which are not bonded with a divalent group represented by the formula (12a), and R₅ to R₈ which are not bonded with a divalent group represented by the formula (12b) is a group represented by the following formula (13);

two R's may be the same or different;

R₃₁ to R₃₅ are independently a hydrogen atom, a substituted or unsubstituted alkyl group including 1 to 50 carbon atoms, a substituted or unsubstituted aryl group including 6 to 50 ring carbon atoms, or a substituted or unsubstituted monovalent heterocyclic group including 5 to 50 ring atoms;

when a plurality of R₃₁ to R₃₅ are present, the plurality of R₃₁ to R₃₅ may be the same or different;

when a plurality of groups represented by the following formula (13) are present, the plurality of groups represented by the following formula (13) may be the same or different;

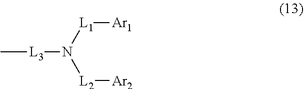

wherein in the formula (13),

L₁ to L₃ are independently a single bond, a substituted or unsubstituted alkylene group including 1 to 30 carbon atoms, a substituted or unsubstituted arylene group including 6 to 30 ring carbon atoms, or a substituted or unsubstituted divalent heterocyclic group including 5 to 30 ring atoms;

Ar₁ and Ar₂ are independently a substituted or unsubstituted alkyl group including 1 to 50 carbon atoms, a substituted or unsubstituted aryl group including 6 to 50 ring carbon atoms or a substituted or unsubstituted monovalent heterocyclic group including 5 to 50 ring atoms, and Ar₁ and Ar₂ may be bonded with each other via a single bond or via —O—,—S— or —C(R)₂—; and

R is a substituent, and two Rs may be the same or different.

The compound represented by the formula (1) (hereinafter may be referred to as the compound (1)) has at its both ends, for example, a naphthalene ring formed by condensation of a divalent group represented by the formula (12a) to a divalent group represented by the formula (11), and a naphthalene ring formed by condensation of a divalent group represented by the formula (12b) to a skeleton represented by the formula (1) and has at least one group represented by the formula (13) (a substituted amino group).

Here, “*” (asterisk) in the formula (11) indicates, for example, a partner which is bonded with R₁ and R₂, R₂ and R₃, R₃ and R₄, which are respectively bonded with the formula (11).

In the formula (11), two “*” are present. For example, when the group represented by the formula (11) is bonded with R₁ and R₂, the two “*” may be bonded with either R₁ or R₂ in the bonding of the formula (1) and the formula (11) as shown in the following formula:

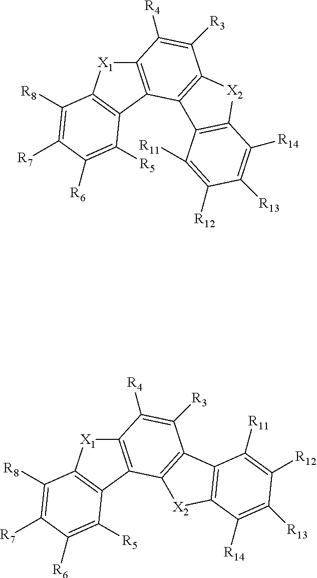

“*” in the formulas (12a) and (12b) has the same meaning as “*” in the formula (11).

“At least one pair among R₁₁ and R₁₂, R₁₂ and R₁₃, and R₁₃ and R₁₄” means that one, two or three pairs are simultaneously bonded with the formula (12a), respectively.

For example, when two pairs of R₁₁ and R₁₂, and R₁₃ and R₁₄ are simultaneously bonded with the formula (12a), the structure represented by the following formula is obtained.

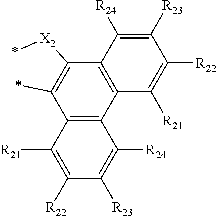

For example, when two pairs of R₁₁ and R₁₂, and R₁₂ and R₁₃ are simultaneously bonded with the formula (12a), and when three pairs of R₁₁ and R₁₂, R₁₂ and R₁₃, and R₁₃ and R₁₄ are simultaneously bonded with the formula (12a), two or three groups represented by the formula (12a) are condensed into the structures represented by the following formulas.

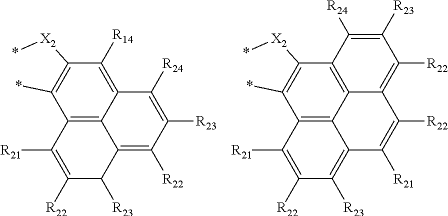

“At least one pair of R₅ and R₆, R₅ and R₇, and R₇ and R₈” means that one, two or three pairs are simultaneously bonded with the formula (12b), and the case when two or three pairs are simultaneously bonded is the same as above-described.

A compound (1) may be represented by the following formulas (1-1) to (1-6) depending on the bonding variation of the formula (11) to a skeleton represented by the formula (1).

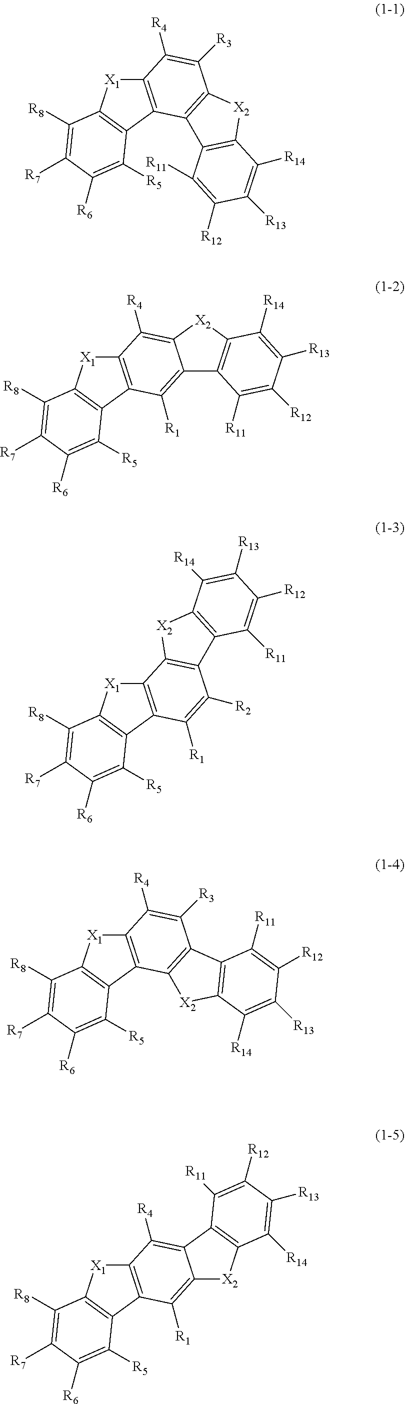

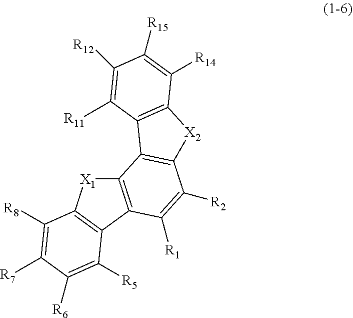

wherein in the formulas (1-1) to (1-6), X₁, X₂, R₁ to R₈ and R₁₁ to R₁₄ are as defined in the formula (1).

In one embodiment, the compound (1) is selected from the group consisting of compounds represented by the following formulas (1-1H) to (1-6H), wherein R₁ to R₄ are hydrogen atoms.

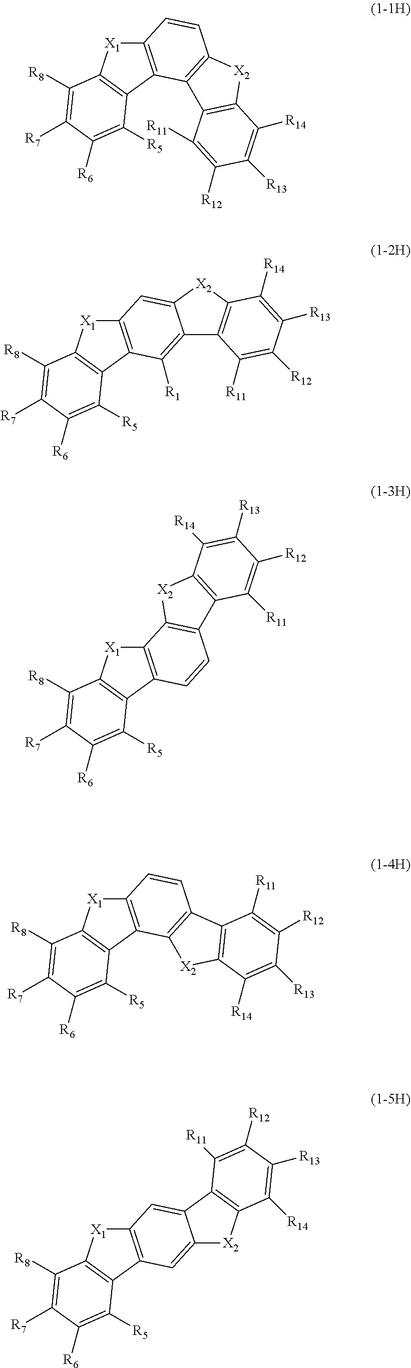

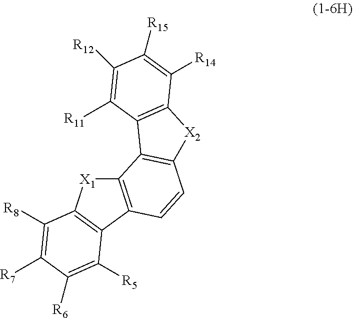

wherein in the formulas (1-1H) to (1-6H), X₁, X₂, R₅ to R₈ and R₁₁ to R₁₄ are as defined in the formula (1).

In one embodiment, it is preferable that the compound (1) be a compound represented by the formula (1-2H).

In one embodiment, dependent on the bonding variation of the formula (12a), the divalent group represented by the formula (11) is selected from the group consisting of divalent groups represented by the formulas (11-1) to (11-3):

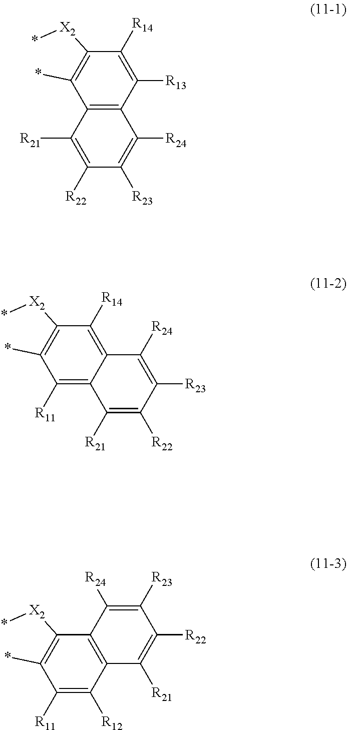

wherein in the formulas (11-1) to (11-3), X₂, R₁₁ to R₁₄ and R₂₁ to R₂₄ are as defined in the formula (1).

In one embodiment, the divalent group represented by the formula (11) is selected from the group consisting of divalent groups represented by the following formulas (11-1H) to (11-3H), wherein R₁₁ to R₁₄ are hydrogen atoms:

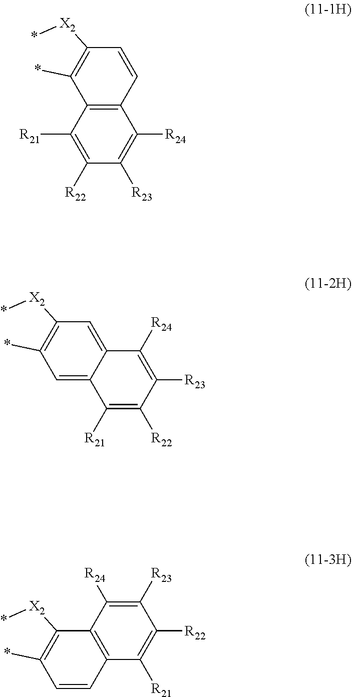

wherein in the formulas (11-1H) to (11-3H), X₂ and R₂₁ to R₂₄ are as defined in the formula (1).

In one embodiment, it is preferable that the group represented by the formula (11) be a divalent group represented by the formula (11-2H).

In one embodiment, the compound (1) is selected from the group consisting of compounds represented by the following formulas (1-21) to (1-23):

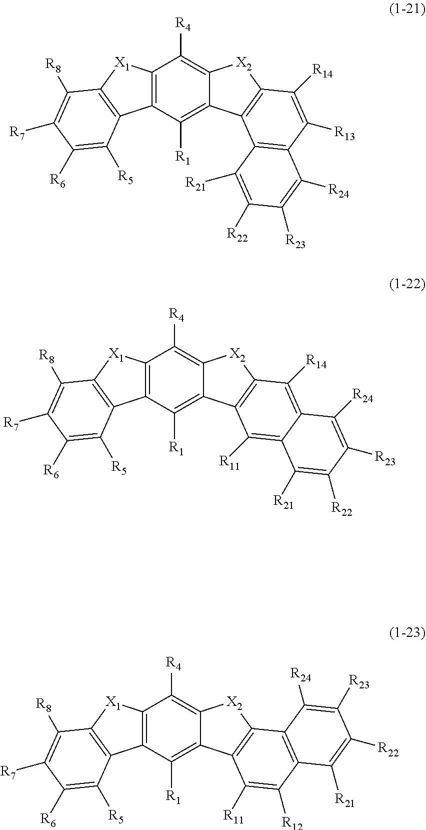

wherein in the formulas (1-21) to (1-23), X₁, X₂, R₁, R₄ to R₈, R₁₁ to R₁₄ and R₂₁ to R₂₄ are as defined in the formula (1).

In one embodiment, the compound (1) is a compound represented by the formula (1-22).

The compound (1) can also be represented by the following formulas (1-11) to (1-13) depending on the bonding variation of the formula (12b) to a skeleton represented by the formula (1):

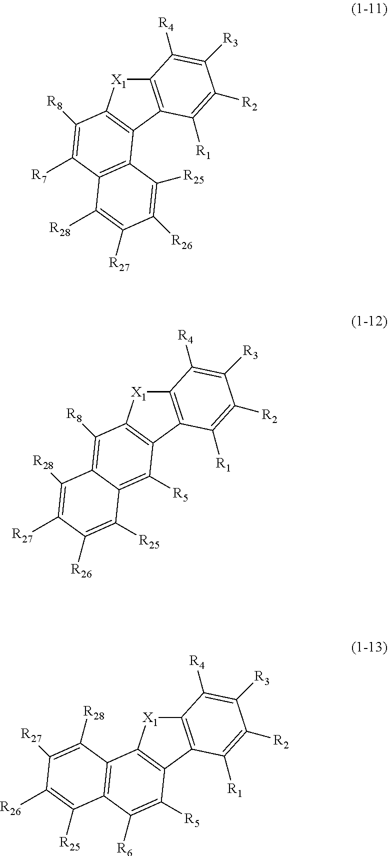

wherein in the formulas (1-11) to (1-13), X₁, R₁ to R₈ and R₂₅ to R₂₈ are as defined in the formula (1).

In one embodiment, the compound (1) is selected from the group consisting of compounds represented by the following formulas (1-11H) to (1-13H), wherein R₅ to R₈ are hydrogen atoms.

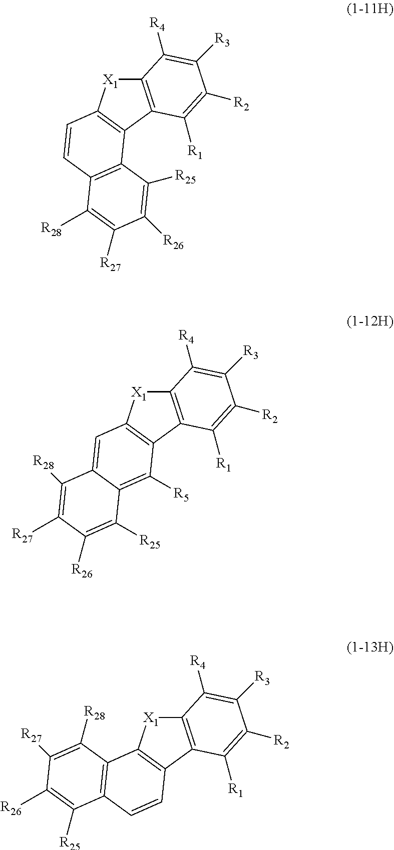

wherein in the formulas (1-11H) to (1-13H), X₁, R₁ to R₄ and R₂₅ to R₂₈ are as defined in the formula (1).

In one embodiment, the compound (1) is a compound represented by the formula (1-12H).

In one embodiment, the compound represented by the formula (1-2) is selected from the group consisting of compounds represented by the following formulas (1-24) to (1-26).

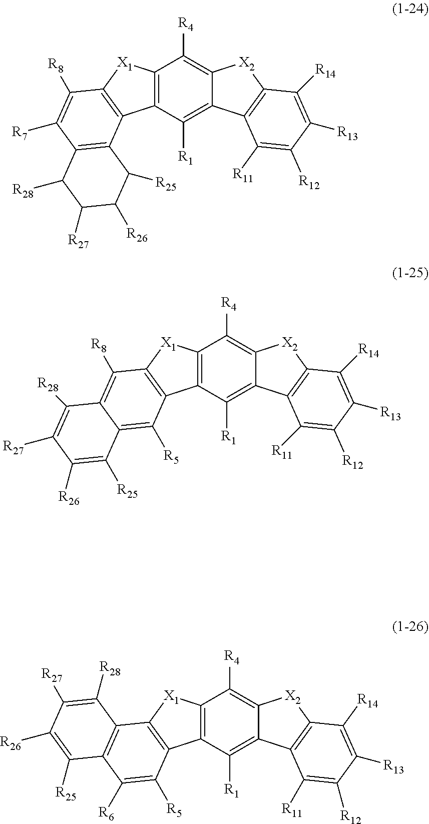

wherein in the formulas (1-24) to (1-26), X₁, X₂, R₁, R₄ to R₈, R₁₁ to R₁₄ and R₂₅ to R₂₈ are as defined in the formula (1).

In one embodiment, the compound represented by the formula (1-2) is a compound represented by the formula (1-25).

In one embodiment, the compound (1) is selected from the group consisting of compounds represented by the following formulas (1-31) to (1-35).

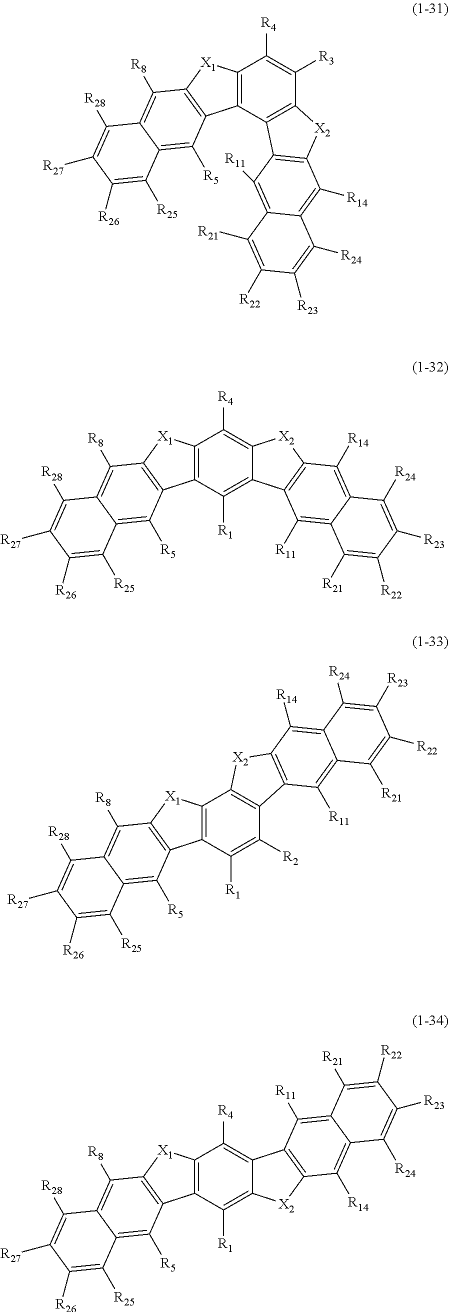

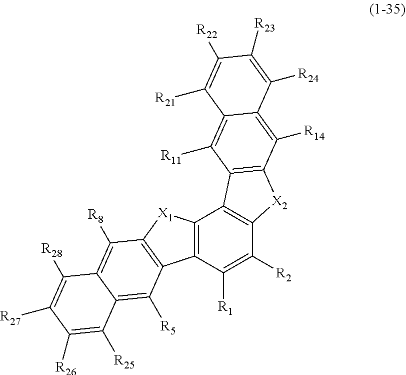

wherein in the formulas (1-31) to (1-35), X₁, X₂, R₁ to R₅, R₈, R₁₁, R₁₄ and R₂₁ to R₂₈ are as defined in the formula (1).

In one embodiment, the compound (1) is a compound represented by the formula (1-32).

In one embodiment, one of R₂₁ to R₂₄ and one of R₂₅ to R₂₈ are independently a group represented by the formula (13).

In one embodiment, the compound represented by the formula (1-32) is a compound represented by the following formula (1-40).

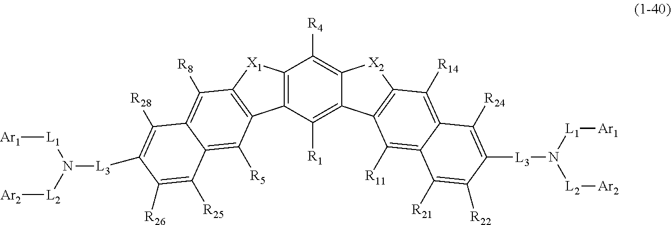

wherein in the formula (1-40), X₁, X₂, R₁, R₄, R₅, R₈, R₁₁, R₁₄, R₂₁, R₂₂, R₂₄ to R₂₆, R₂₆, L₁ to L₃, Ar₁ and Ar₂ are as defined in the formula (1); and

a plurality of L₁ to L₃, Ar₁ and Ar₂ may be the same or different, respectively.

In one embodiment, the compound represented by the formulas (1-31) and (1-33) to (1-35) is a compound represented by the following formulas (1-41) to (1-44).

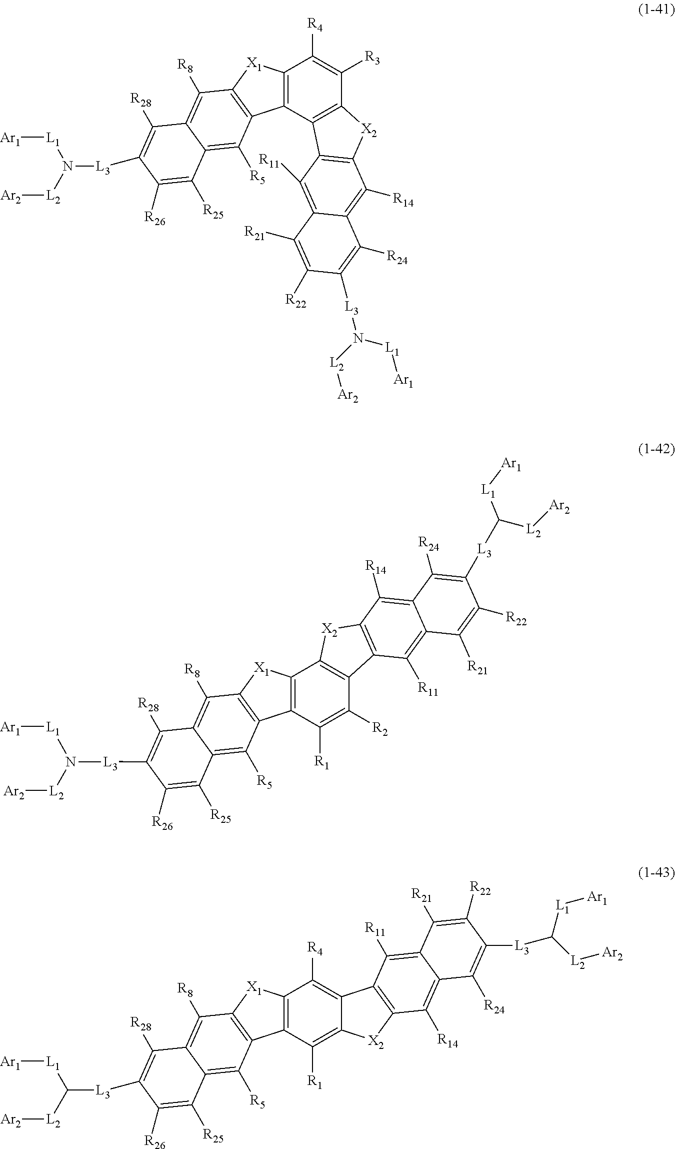

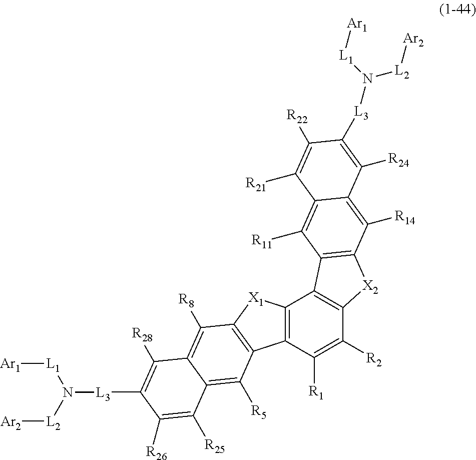

wherein in the formulas (1-41) to (1-44), X₁, X₂, R₁ to R₅, R₈, R₁₁, R₁₄, R₂₁, R₂₂, R₂₄ to R₂₆, R₂₈, L₁ to L₃, Ar₁ and Ar₂ are as defined in the formula (1); and

a plurality of L₁ to L₃, Ar₁ and Ar₂ may be the same or different, respectively.

In one embodiment, X₁ and X₂ are O (an oxygen atom).

In one embodiment, two R′ are independently a hydrogen atom or a methyl group.

In one embodiment, among R₂₁ to R₂₈, R₁ to R₄ which are not bonded with a divalent group represented by the formula (11), R₁ to R₁₄ which are not bonded to a divalent group represented by the formula (12a), and R₅ to R₈ which are not bonded to a divalent group represented by the formula (12b), moieties other than the group represented by the formula (13) are hydrogen atoms.

In one embodiment, L₃ is a single bond.

In one embodiment, Ar₁ and Ar₂ are independently a substituted or unsubstituted aryl group including 6 to 50 ring carbon atoms.

In one embodiment, one of Ar₁ and Ar₂ is a substituted or unsubstituted aryl group including 6 to 50 ring carbon atoms and the other of Ar₁ and Ar₂ is a substituted or unsubstituted monovalent heterocyclic group including 5 to 50 ring atoms.

In one embodiment, the group represented by the formula (13) is selected from the group consisting of groups represented by the following formulas (13-1) to (13-3).

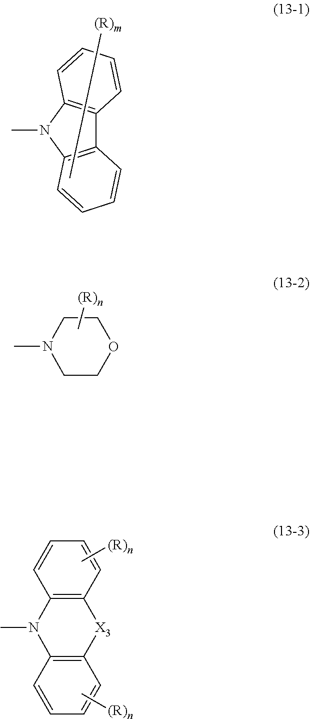

wherein in the formulas (13-1) to (13-3), R is a substituent, m is an integer of 0 to 8, n is an integer of 0 to 4, and when m or n is 2 or more, a plurality of R may be the same or different; and

X₃ in the formula (13-3) is —O—, —S— or —C(R)₂—, and two Rs may be the same or different.

The formula (13-1) represents a compound, wherein in the formula (13), L₁ to L₃ are single bonds, and Ar₁ and Ar₂ are phenyl groups and bonded with each other via a single bond to form a carbazole ring.

The formula (13-2) represents a compound, wherein in the formula (13), L₁ to L₃ are single bonds, and Ar₁ and Ar₂ are ethyl groups and bonded with each other via —O— to form a morpholine ring.

The formula (13-3) represents a compound, wherein in the formula (13), L₁ to L₃ are single bonds, and Ar₁ and Ar₂ are phenyl groups and bonded with each other via X₃ to form a six-membered ring.

The substituent in the case of “substituted or unsubstituted” in the compound (1) (hereinafter referred to as an arbitrary substituent) and the substituent represented by R are selected from the group consisting of an alkyl group including 1 to 50 carbon atoms, a haloalkyl group including 1 to 50 carbon atoms, an alkenyl group including 2 to 50 carbon atoms, an alkynyl group including 2 to 50 carbon atoms, a cycloalkyl group including 3 to 50 ring carbon atoms, an alkoxy group including 1 to 50 carbon atoms, an alkylthio group including 1 to 50 carbon atoms, an aryloxy group including 6 to 50 ring carbon atoms, an arylthio group including 6 to 50 ring carbon atoms, an aralkyl group including 7 to 50 carbon atoms, —Si(R₄₁)(R₄₂)(R₄₃), —C(═O)R₄₄, —COOR₄₅, —S(═O)₂R₄₆, —P(═O)(R₄₇)(R₄₈), —Ge(R)(R₅₀)(R₅₁), —N(R₅₂)(R₅₃), a hydroxy group, a halogen atom, a cyano group, a nitro group, an aryl group including 6 to 50 ring carbon atoms, and a heterocyclic group including 5 to 50 ring atoms, provided that R₄₁ to R₅₃ are independently a hydrogen atom, an alkyl group including 1 to 50 carbon atoms, an aryl group including 6 to 50 ring carbon atoms, or a heterocyclic group including 5 to 50 ring atoms, and provided that, when two or more of each of R₄₁ to R₅₃ are present, the two or more of each of R₄₁ to R₅₃ may be the same or different.

In one embodiment, the substituent in the case of “substituted or unsubstituted” in the compound (1) and the substituent represented by R are selected from the group consisting of an alkyl group including 1 to 50 carbon atoms, an aryl group including 6 to 50 ring carbon atoms and a heterocyclic group including 5 to 50 ring atoms.

In one embodiment, the substituent in the case of “substituted or unsubstituted” in the compound (1) and the substituent represented by R are selected from the group consisting of an alkyl group including 1 to 18 carbon atoms, an aryl group including 6 to 18 ring carbon atoms and a heterocyclic group including 5 to 18 ring atoms.

Specific examples of the substituent, the arbitrary substituent and the halogen atom of the compound (1) are the same as those described above.

Specific examples of the compound (1) include, for example, the following compounds.

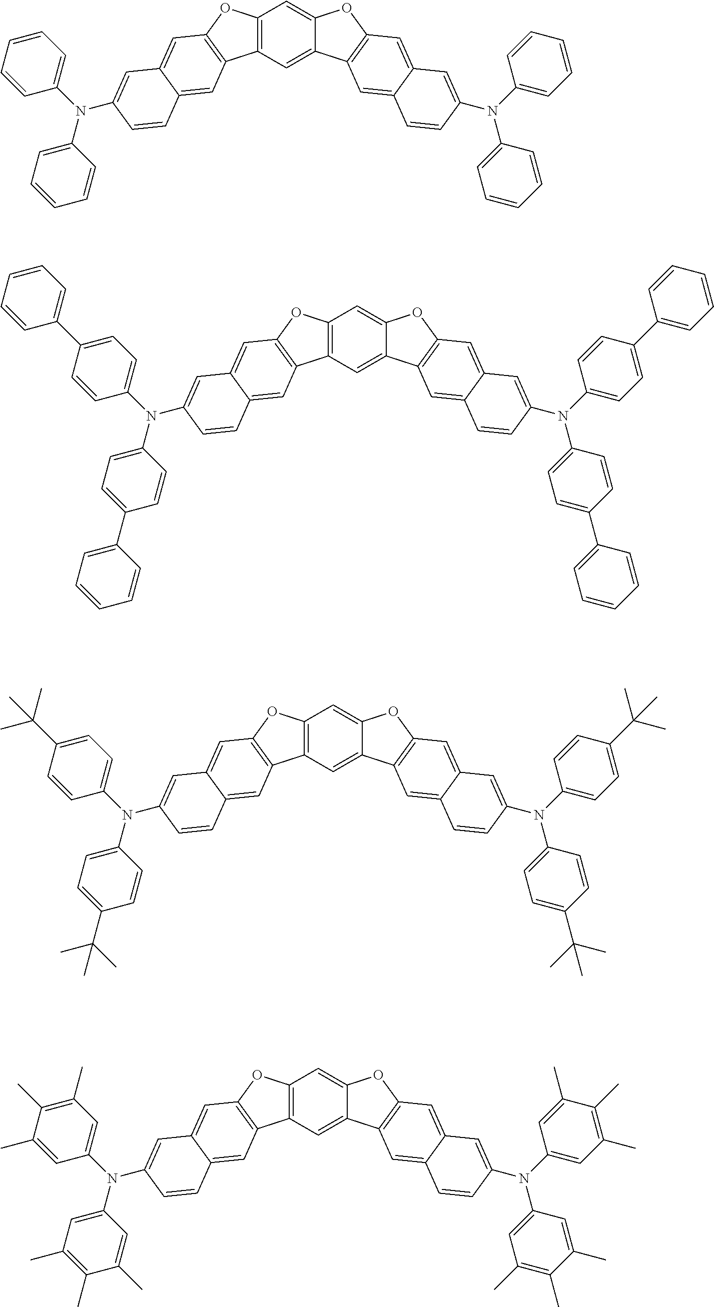

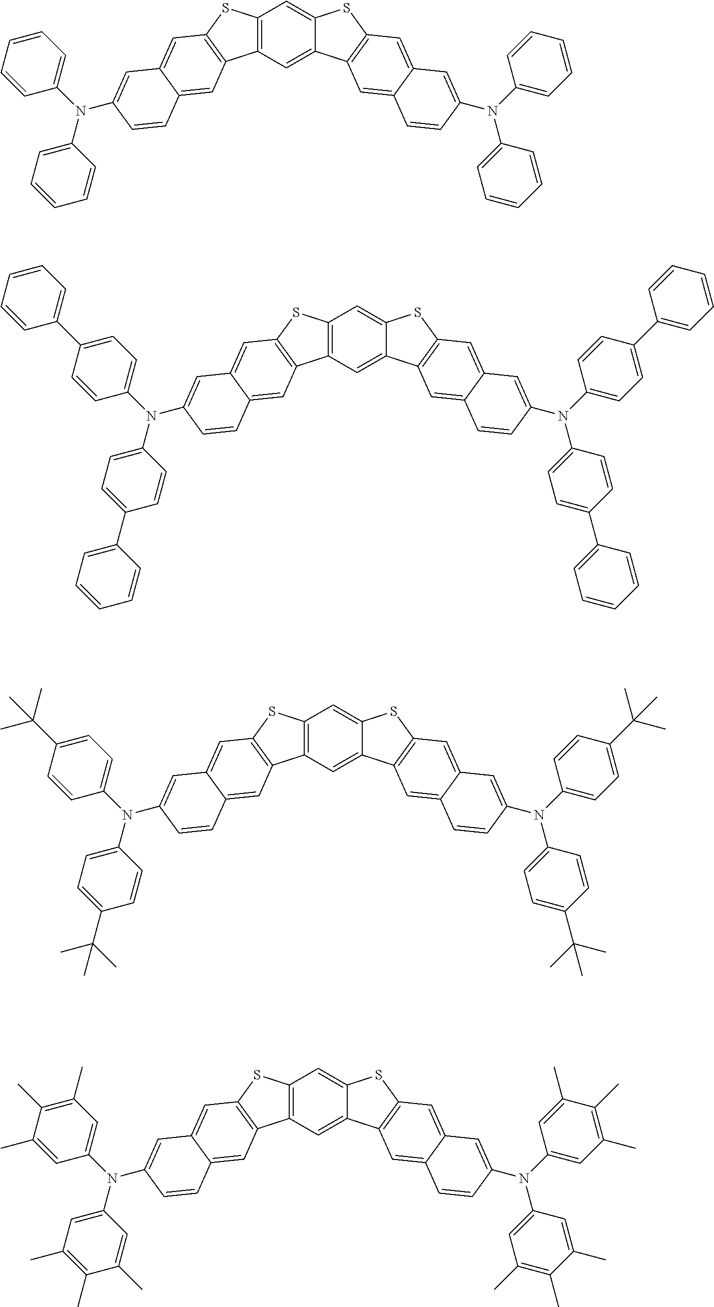

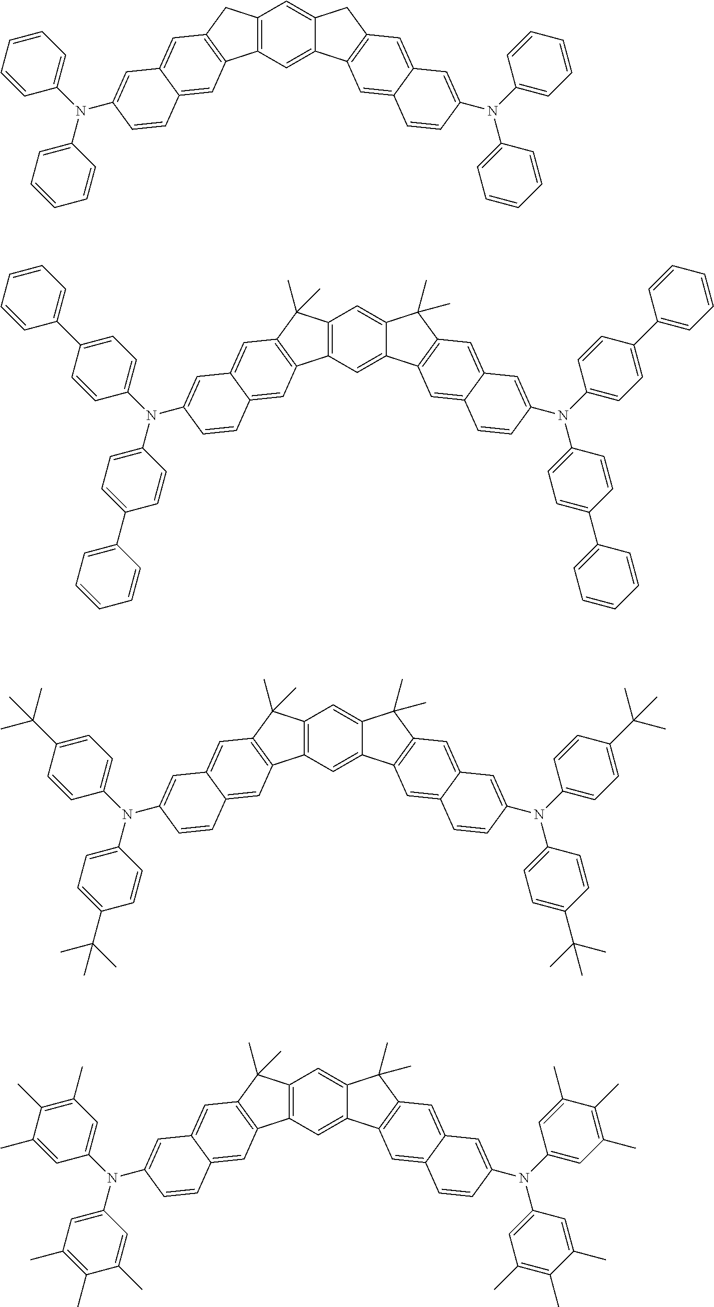

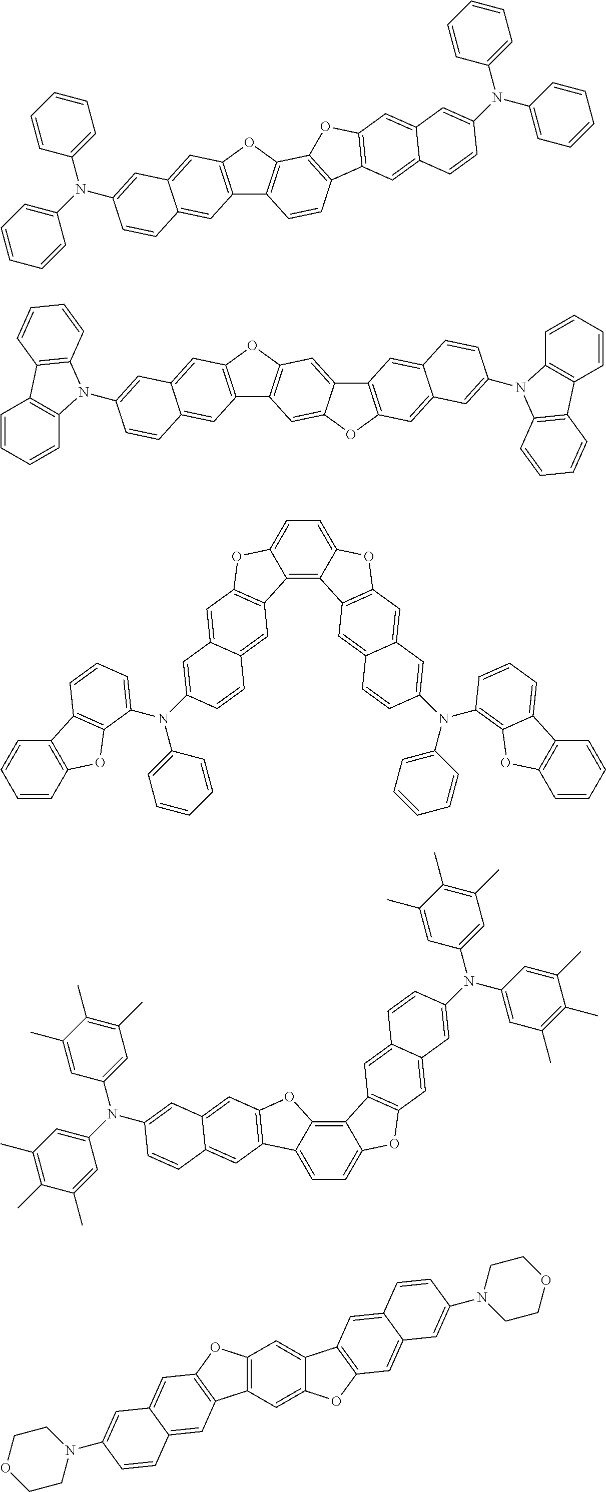

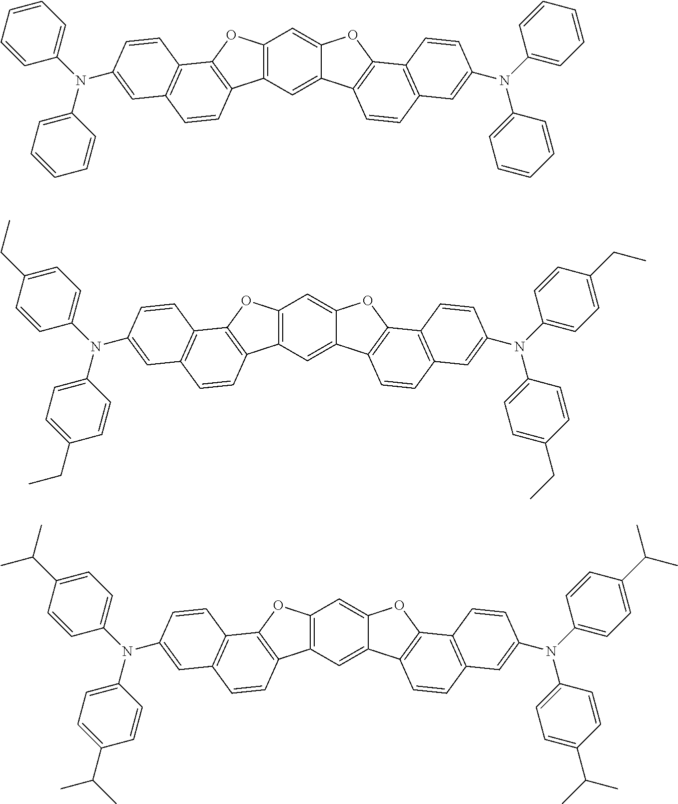

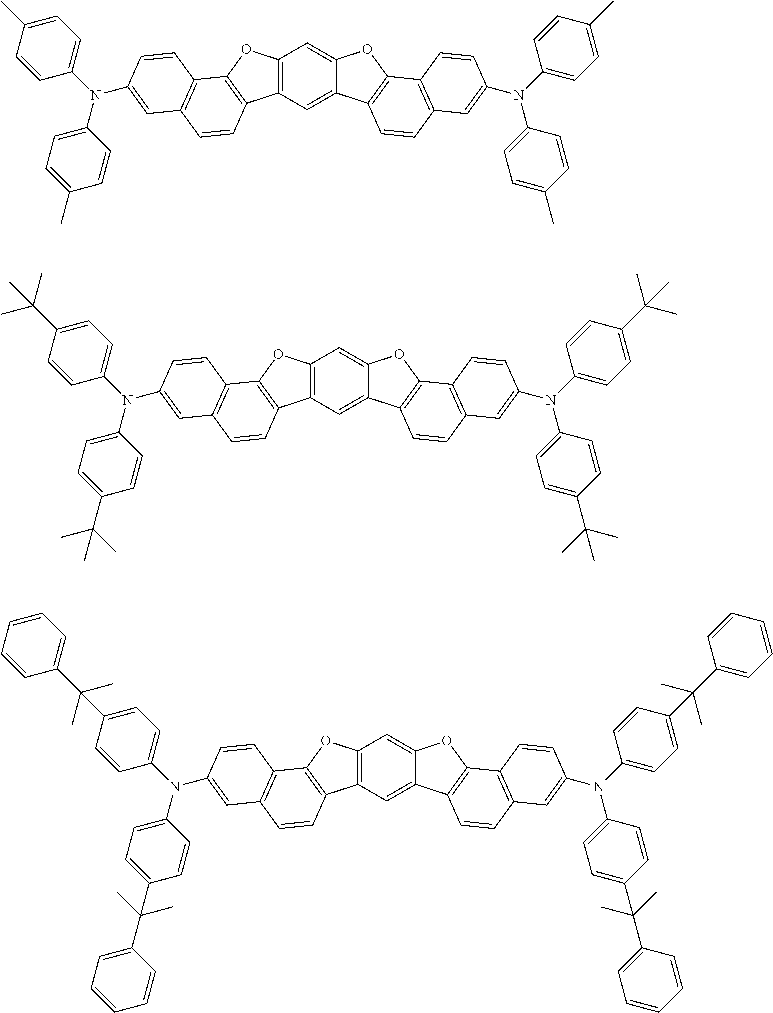

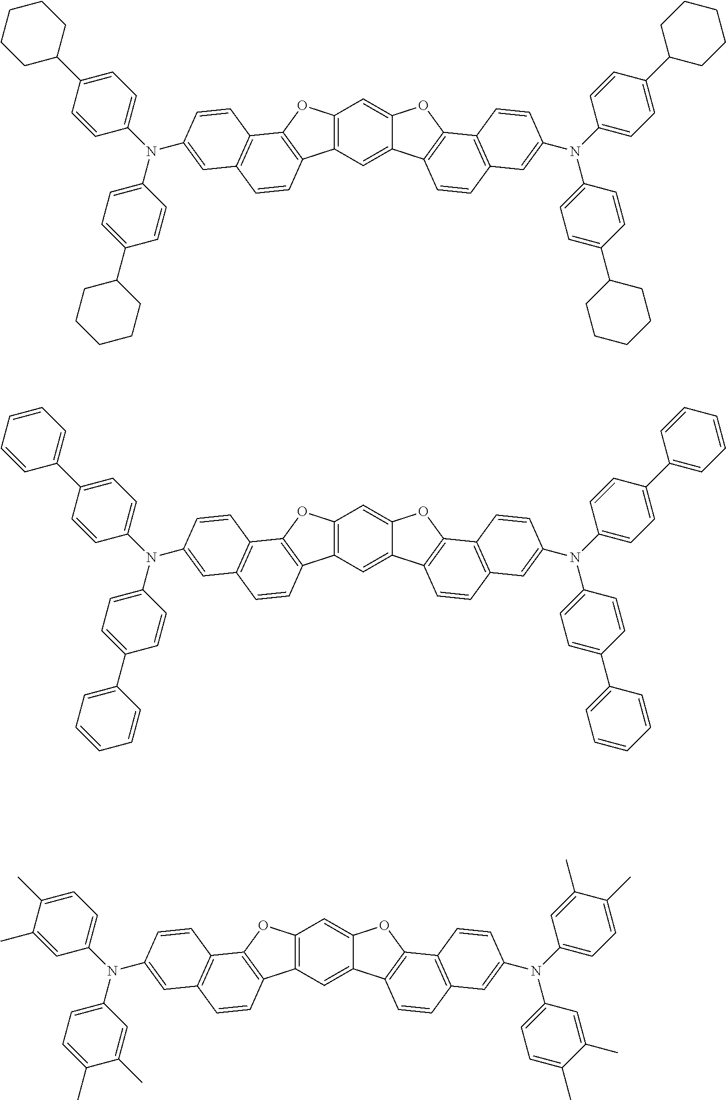

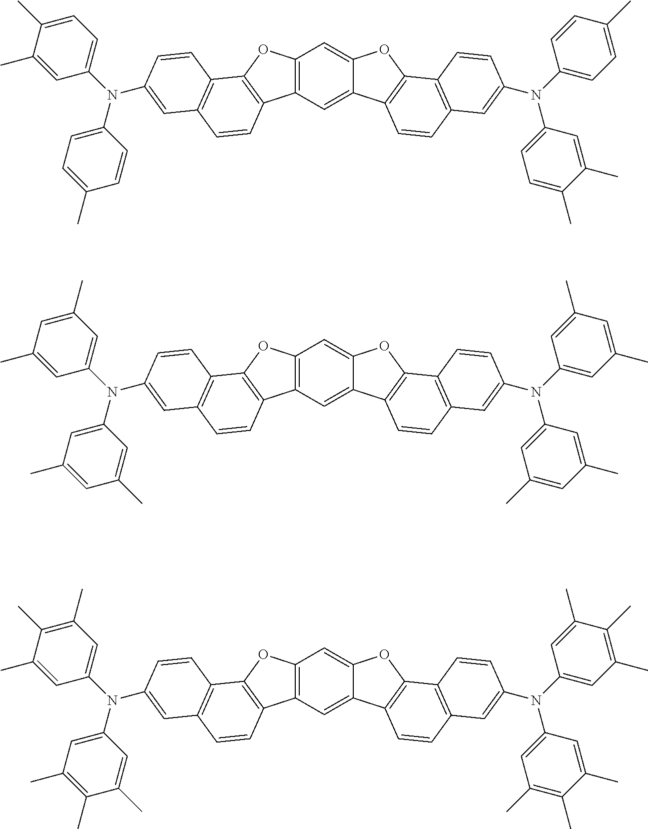

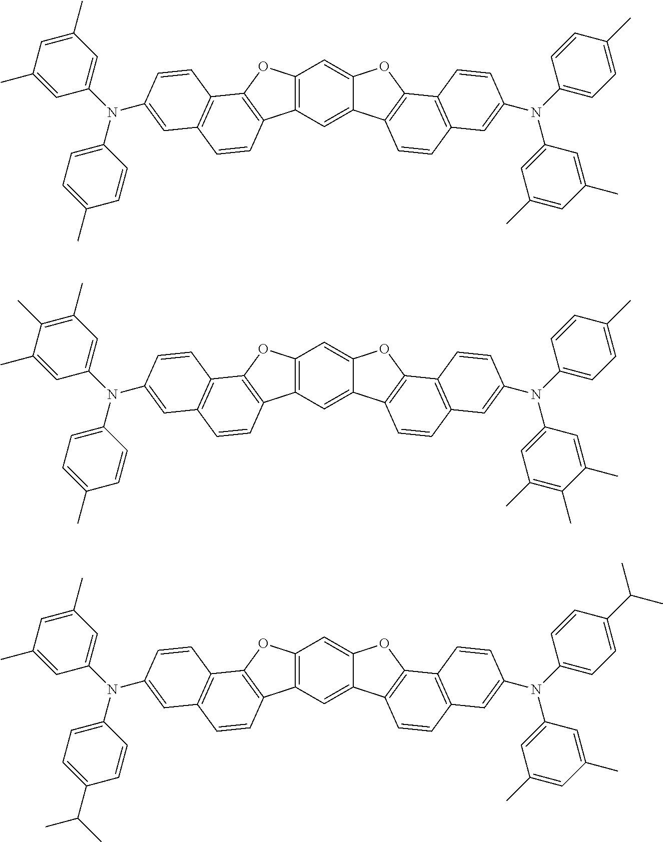

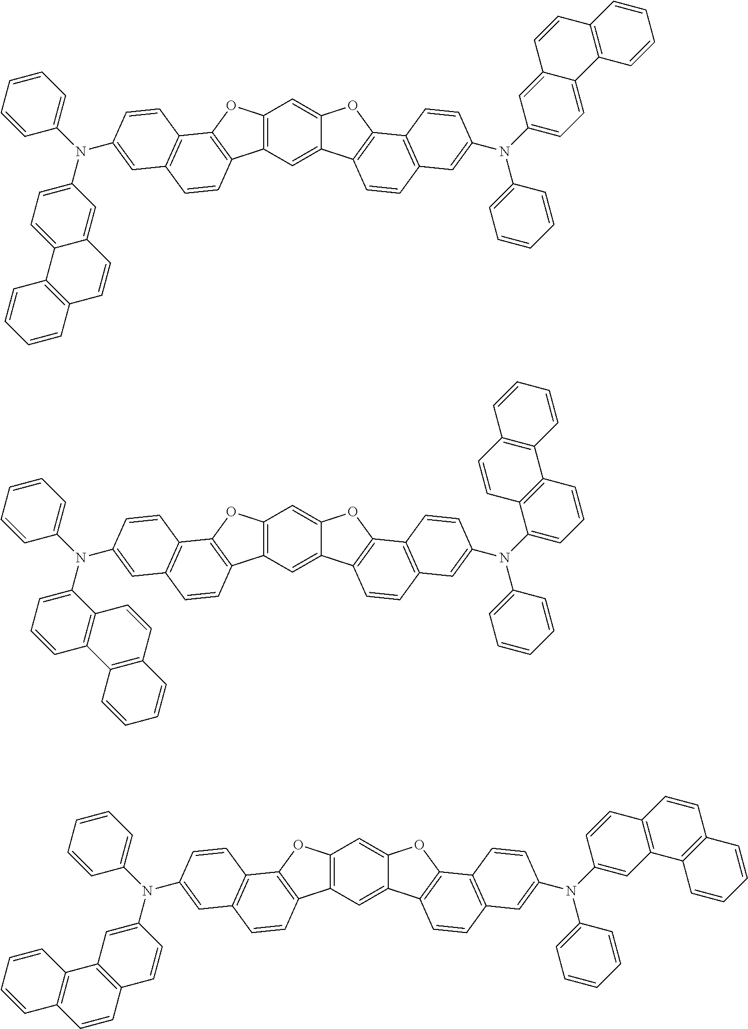 [SMILES: c1ccc(N(c2ccc3c(ccc4ccccc43)c2)c2ccc3c(ccc4c5cc6c(cc5oc34)oc3c4ccc(N(c5ccccc5)c5ccc7c(ccc8ccccc87)c5)cc4ccc63)c2)cc1 ; c1ccc(N(c2ccc3c(ccc4c5cc6c(cc5oc34)oc3c4ccc(N(c5ccccc5)c5cccc7c5ccc5ccccc57)cc4ccc63)c2)c2cccc3c2ccc2ccccc23)cc1 ; c1ccc(N(c2ccc3c(ccc4c5cc6c(cc5oc34)oc3c4ccc(N(c5ccccc5)c5ccc7ccc8ccccc8c7c5)cc4ccc63)c2)c2ccc3ccc4ccccc4c3c2)cc1]

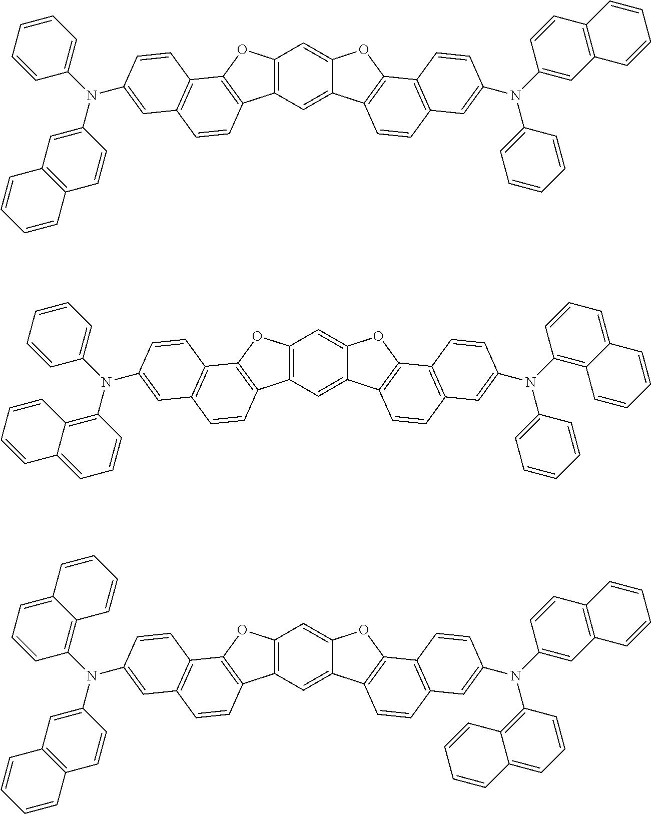 [SMILES: c1ccc(N(c2ccc3ccccc3c2)c2ccc3c(ccc4c5cc6c(cc5oc34)oc3c4ccc(N(c5ccccc5)c5ccc7ccccc7c5)cc4ccc63)c2)cc1 ; c1ccc(N(c2ccc3c(ccc4c5cc6c(cc5oc34)oc3c4ccc(N(c5ccccc5)c5cccc7ccccc57)cc4ccc63)c2)c2cccc3ccccc23)cc1 ; c1ccc2cc(N(c3ccc4c(ccc5c6cc7c(cc6oc45)oc4c5ccc(N(c6ccc8ccccc8c6)c6cccc8ccccc68)cc5ccc74)c3)c3cccc4ccccc34)ccc2c1]

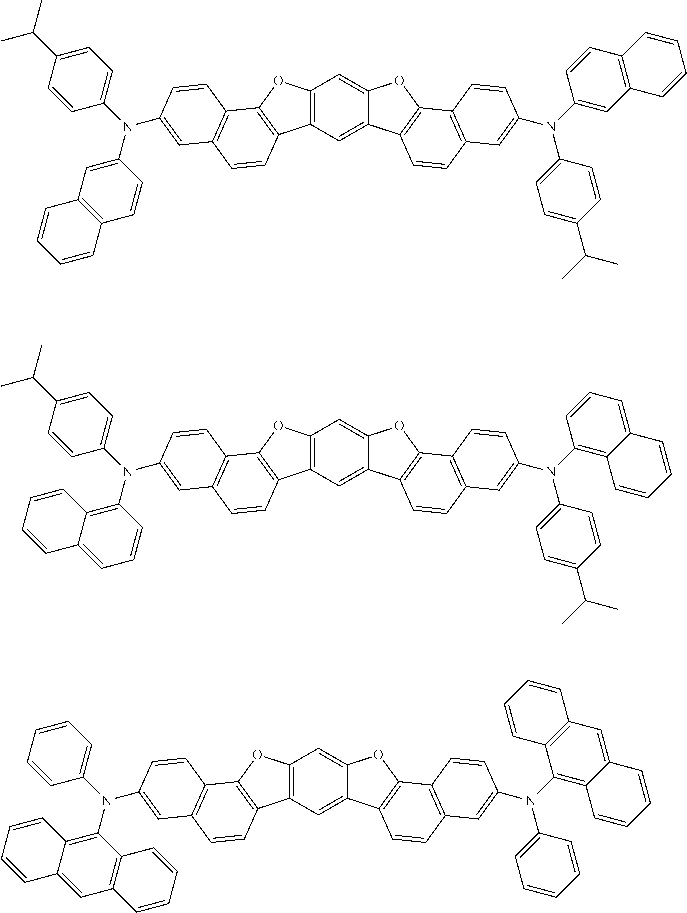 [SMILES: CC(C)c1ccc(N(c2ccc3ccccc3c2)c2ccc3c(ccc4c5cc6c(cc5oc34)oc3c4ccc(N(c5ccc(C(C)C)cc5)c5ccc7ccccc7c5)cc4ccc63)c2)cc1 ; CC(C)c1ccc(N(c2ccc3c(ccc4c5cc6c(cc5oc34)oc3c4ccc(N(c5ccc(C(C)C)cc5)c5cccc7ccccc57)cc4ccc63)c2)c2cccc3ccccc23)cc1 ; c1ccc(N(c2ccc3c(ccc4c5cc6c(cc5oc34)oc3c4ccc(N(c5ccccc5)c5c7ccccc7cc7ccccc57)cc4ccc63)c2)c2c3ccccc3cc3ccccc23)cc1]

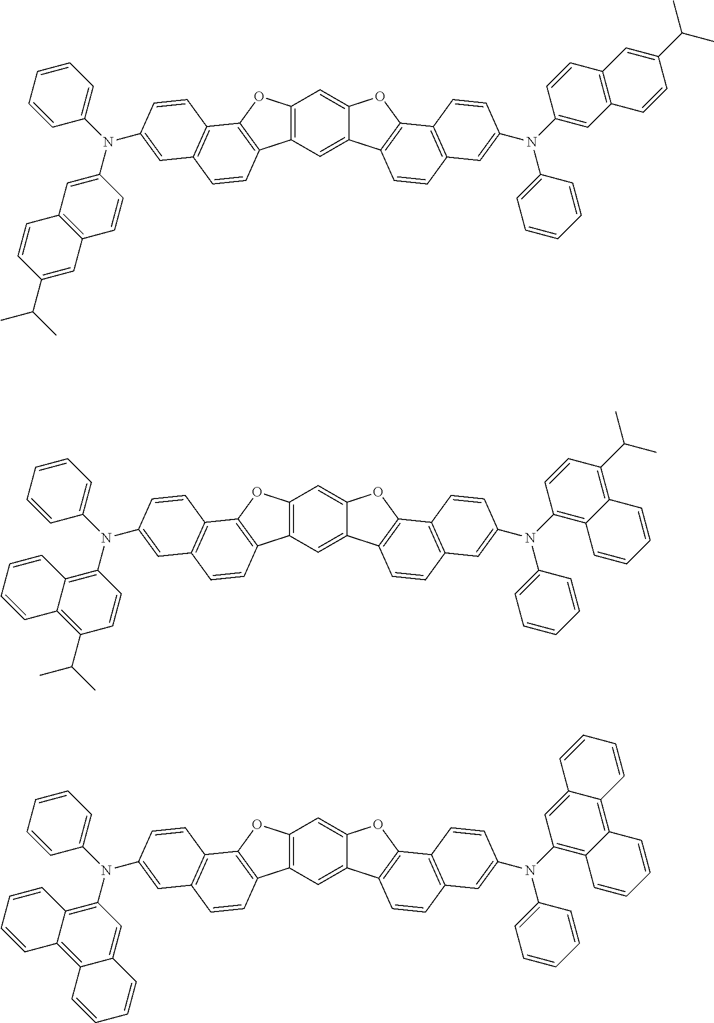 [SMILES: CC(C)c1ccc2cc(N(c3ccccc3)c3ccc4c(ccc5c6cc7c(cc6oc45)oc4c5ccc(N(c6ccccc6)c6ccc8cc(C(C)C)ccc8c6)cc5ccc74)c3)ccc2c1 ; CC(C)c1ccc(N(c2ccccc2)c2ccc3c(ccc4c5cc6c(cc5oc34)oc3c4ccc(N(c5ccccc5)c5ccc(C(C)C)c7ccccc57)cc4ccc63)c2)c2ccccc12 ; c1ccc(N(c2ccc3c(ccc4c5cc6c(cc5oc34)oc3c4ccc(N(c5ccccc5)c5cc7ccccc7c7ccccc57)cc4ccc63)c2)c2cc3ccccc3c3ccccc23)cc1]

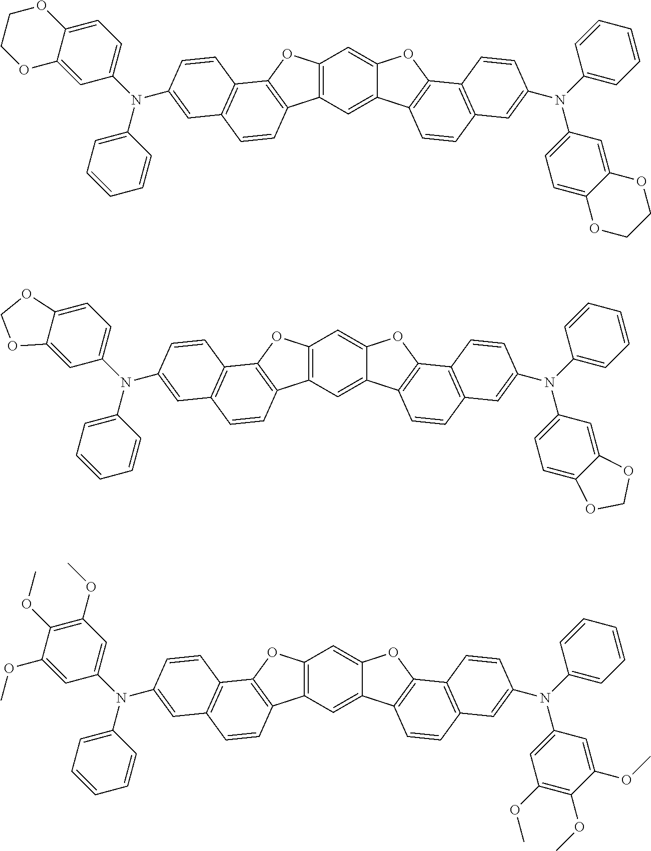

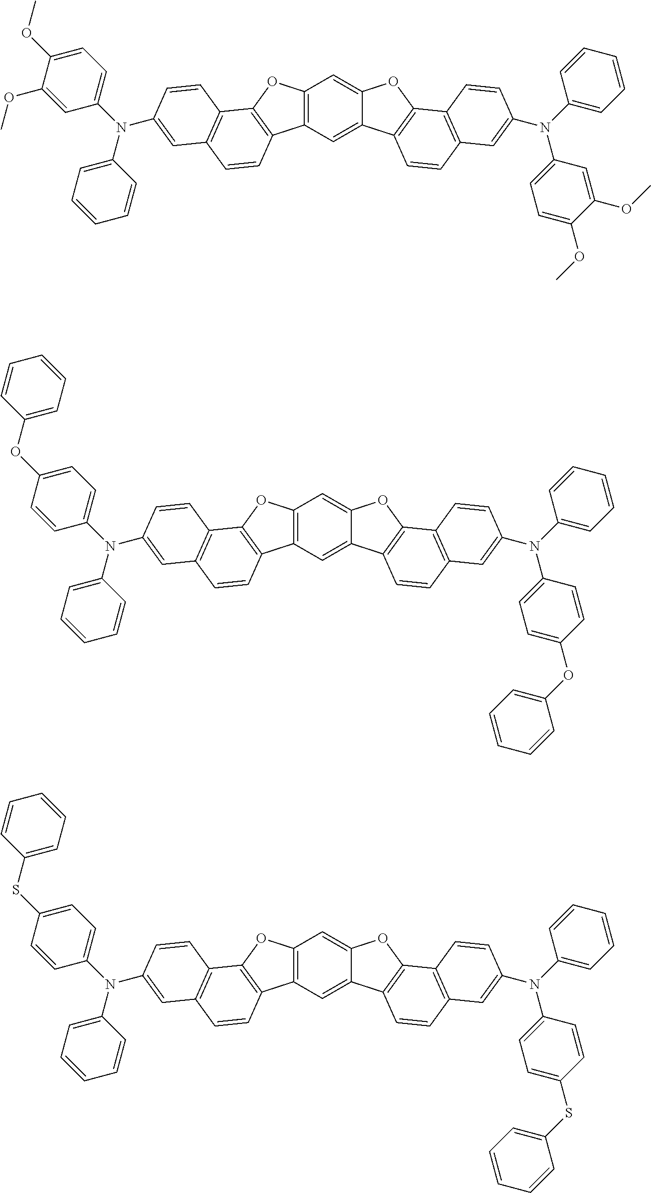

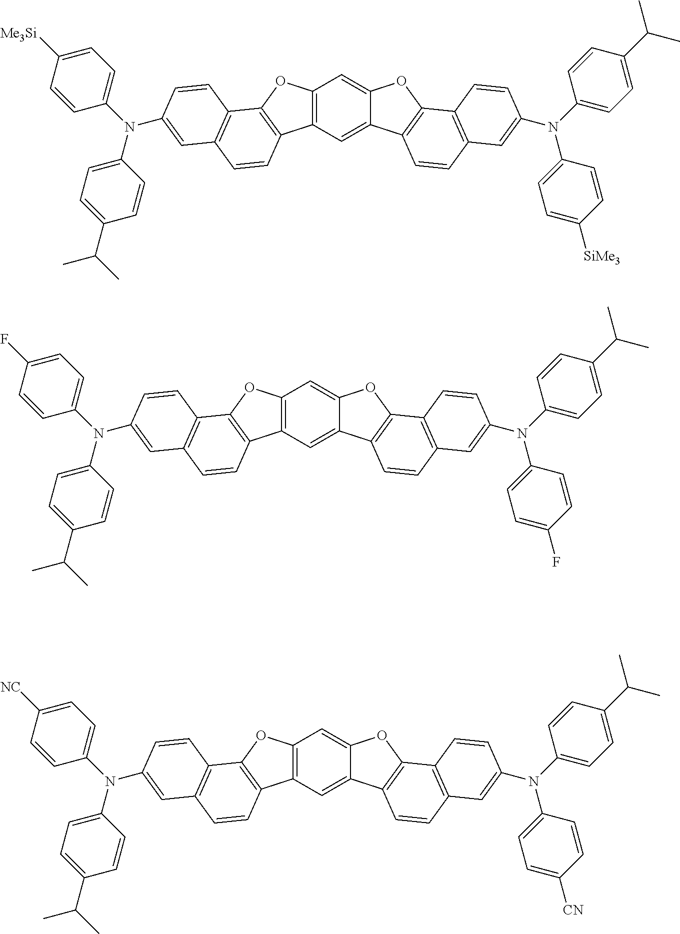 [SMILES: CC(C)c1ccc(N(c2ccc([Si](C)(C)C)cc2)c2ccc3c(ccc4c5cc6c(cc5oc34)oc3c4ccc(N(c5ccc(C(C)C)cc5)c5ccc([Si](C)(C)C)cc5)cc4ccc63)c2)cc1 ; CC(C)c1ccc(N(c2ccc(F)cc2)c2ccc3c(ccc4c5cc6c(cc5oc34)oc3c4ccc(N(c5ccc(F)cc5)c5ccc(C(C)C)cc5)cc4ccc63)c2)cc1 ; CC(C)c1ccc(N(c2ccc(C#N)cc2)c2ccc3c(ccc4c5cc6c(cc5oc34)oc3c4ccc(N(c5ccc(C#N)cc5)c5ccc(C(C)C)cc5)cc4ccc63)c2)cc1]

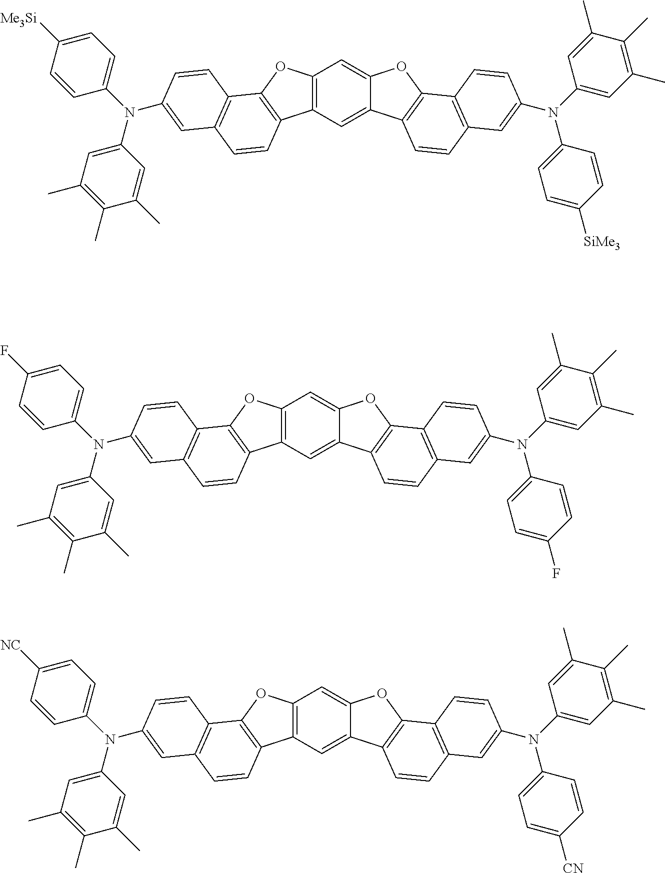 [SMILES: Cc1cc(N(c2ccc([Si](C)(C)C)cc2)c2ccc3c(ccc4c5cc6c(cc5oc34)oc3c4ccc(N(c5ccc([Si](C)(C)C)cc5)c5cc(C)c(C)c(C)c5)cc4ccc63)c2)cc(C)c1C ; Cc1cc(N(c2ccc(F)cc2)c2ccc3c(ccc4c5cc6c(cc5oc34)oc3c4ccc(N(c5ccc(F)cc5)c5cc(C)c(C)c(C)c5)cc4ccc63)c2)cc(C)c1C ; Cc1cc(N(c2ccc(C#N)cc2)c2ccc3c(ccc4c5cc6c(cc5oc34)oc3c4ccc(N(c5ccc(C#N)cc5)c5cc(C)c(C)c(C)c5)cc4ccc63)c2)cc(C)c1C]

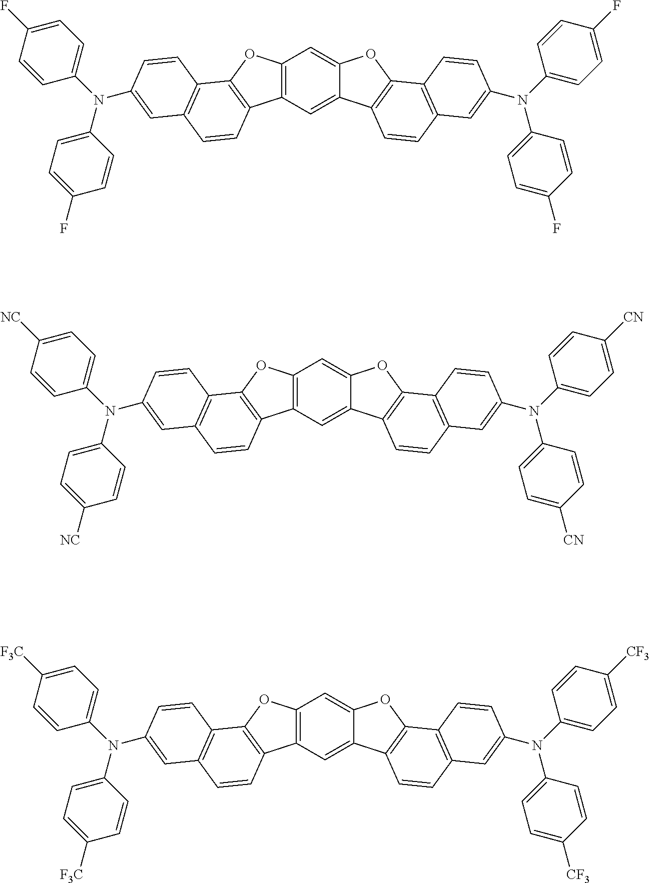 [SMILES: Fc1ccc(N(c2ccc(F)cc2)c2ccc3c(ccc4c5cc6c(cc5oc34)oc3c4ccc(N(c5ccc(F)cc5)c5ccc(F)cc5)cc4ccc63)c2)cc1 ; N#Cc1ccc(N(c2ccc(C#N)cc2)c2ccc3c(ccc4c5cc6c(cc5oc34)oc3c4ccc(N(c5ccc(C#N)cc5)c5ccc(C#N)cc5)cc4ccc63)c2)cc1 ; FC(F)(F)c1ccc(N(c2ccc(C(F)(F)F)cc2)c2ccc3c(ccc4c5cc6c(cc5oc34)oc3c4ccc(N(c5ccc(C(F)(F)F)cc5)c5ccc(C(F)(F)F)cc5)cc4ccc63)c2)cc1]

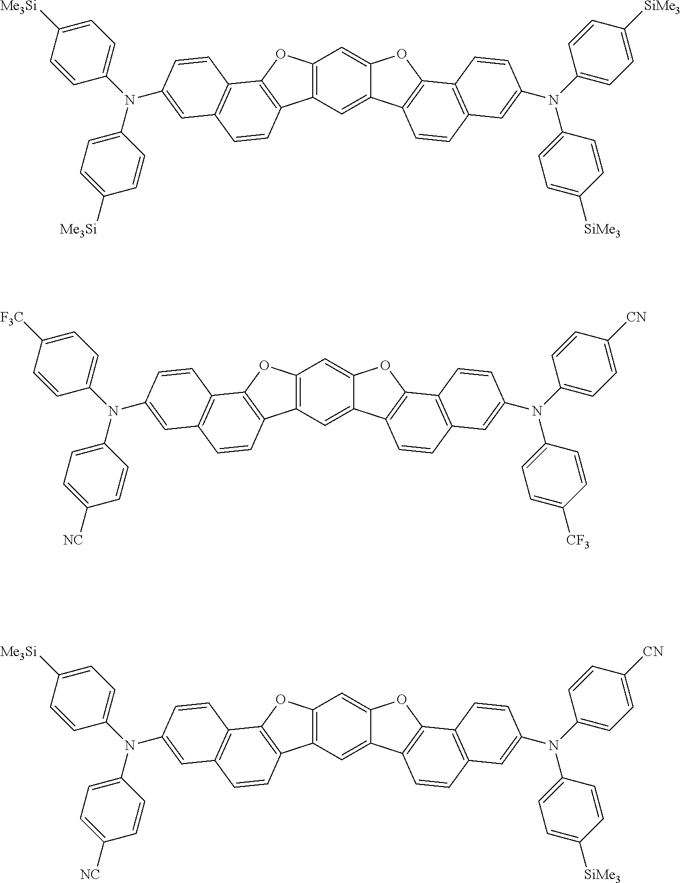 [SMILES: C[Si](C)(C)c1ccc(N(c2ccc([Si](C)(C)C)cc2)c2ccc3c(ccc4c5cc6c(cc5oc34)oc3c4ccc(N(c5ccc([Si](C)(C)C)cc5)c5ccc([Si](C)(C)C)cc5)cc4ccc63)c2)cc1 ; N#Cc1ccc(N(c2ccc(C(F)(F)F)cc2)c2ccc3c(ccc4c5cc6c(cc5oc34)oc3c4ccc(N(c5ccc(C#N)cc5)c5ccc(C(F)(F)F)cc5)cc4ccc63)c2)cc1 ; C[Si](C)(C)c1ccc(N(c2ccc(C#N)cc2)c2ccc3c(ccc4c5cc6c(cc5oc34)oc3c4ccc(N(c5ccc(C#N)cc5)c5ccc([Si](C)(C)C)cc5)cc4ccc63)c2)cc1]

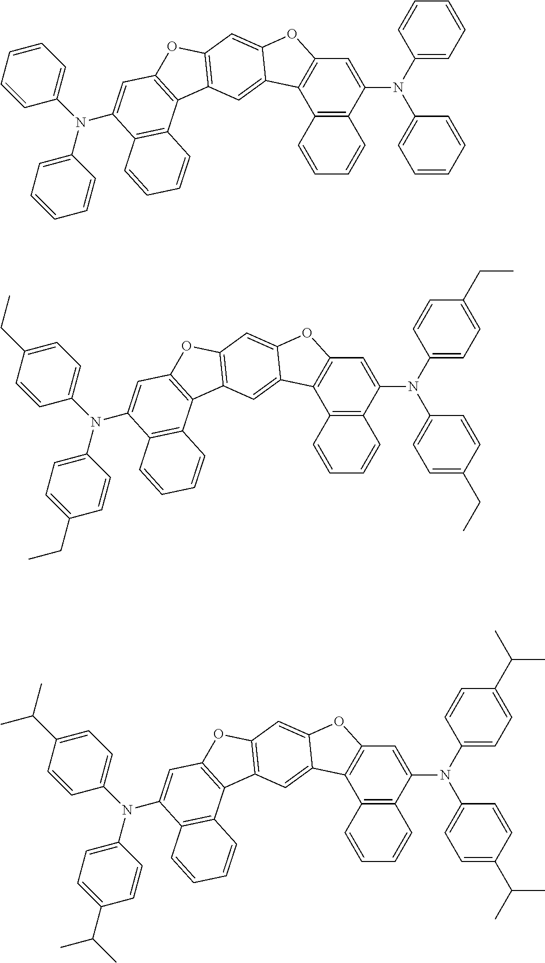

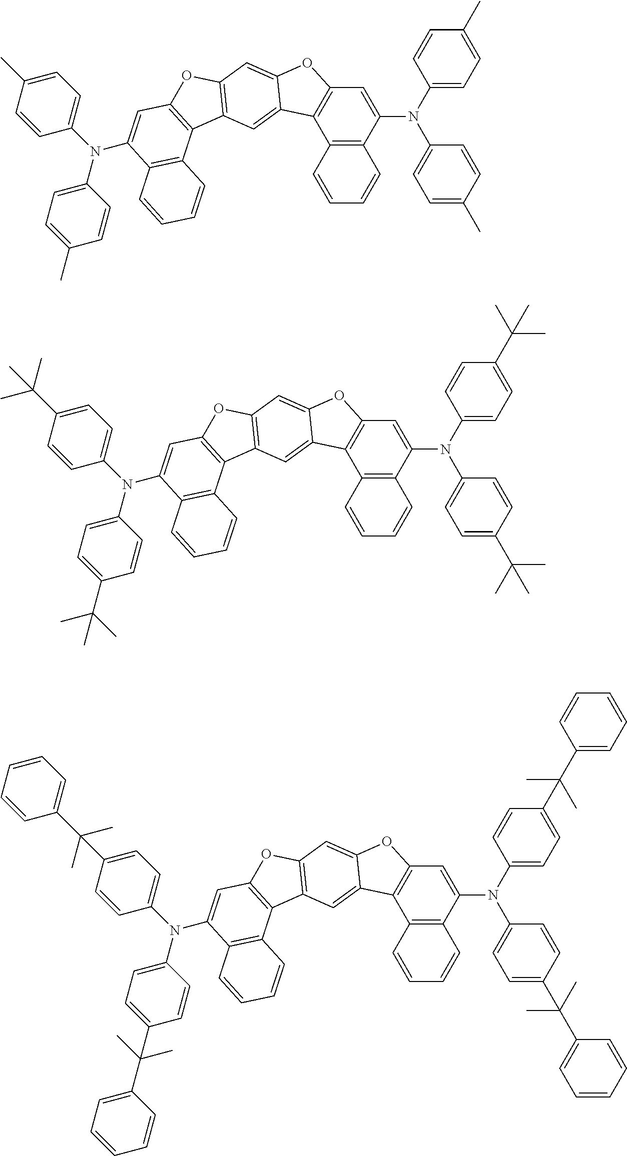

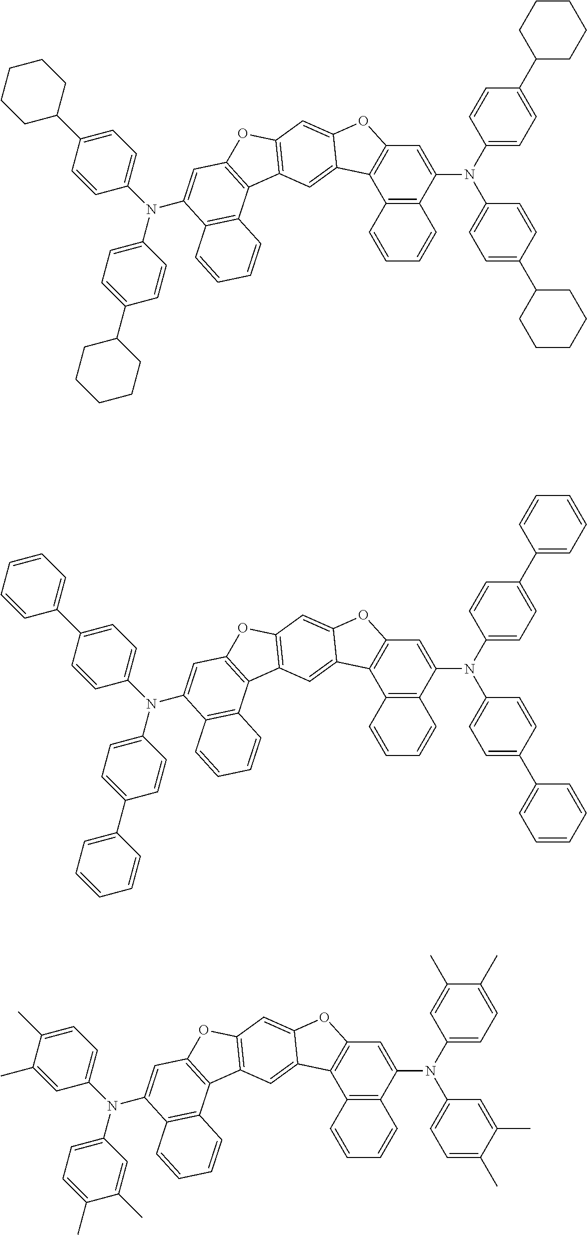

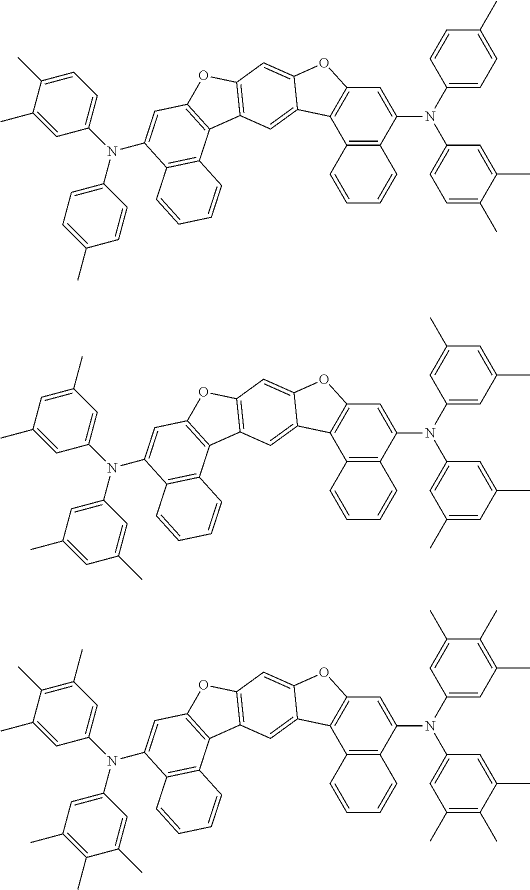

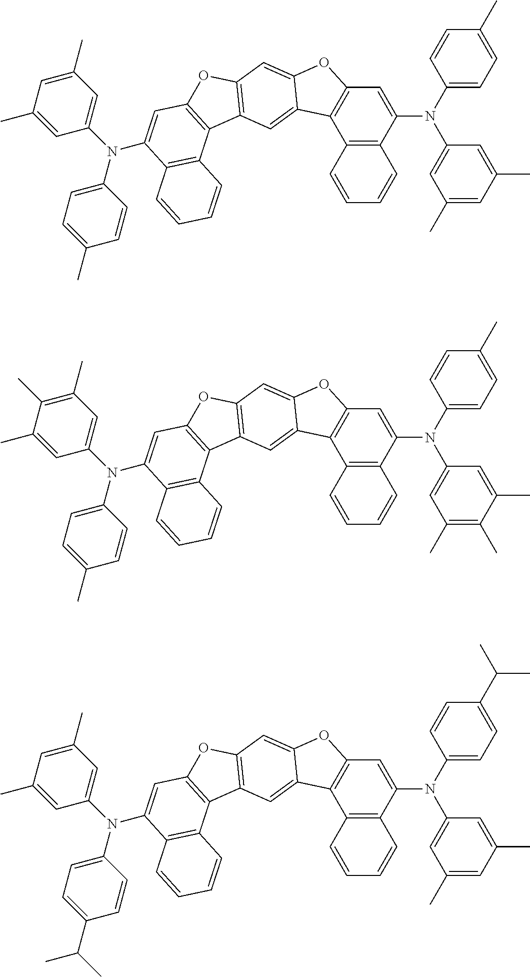

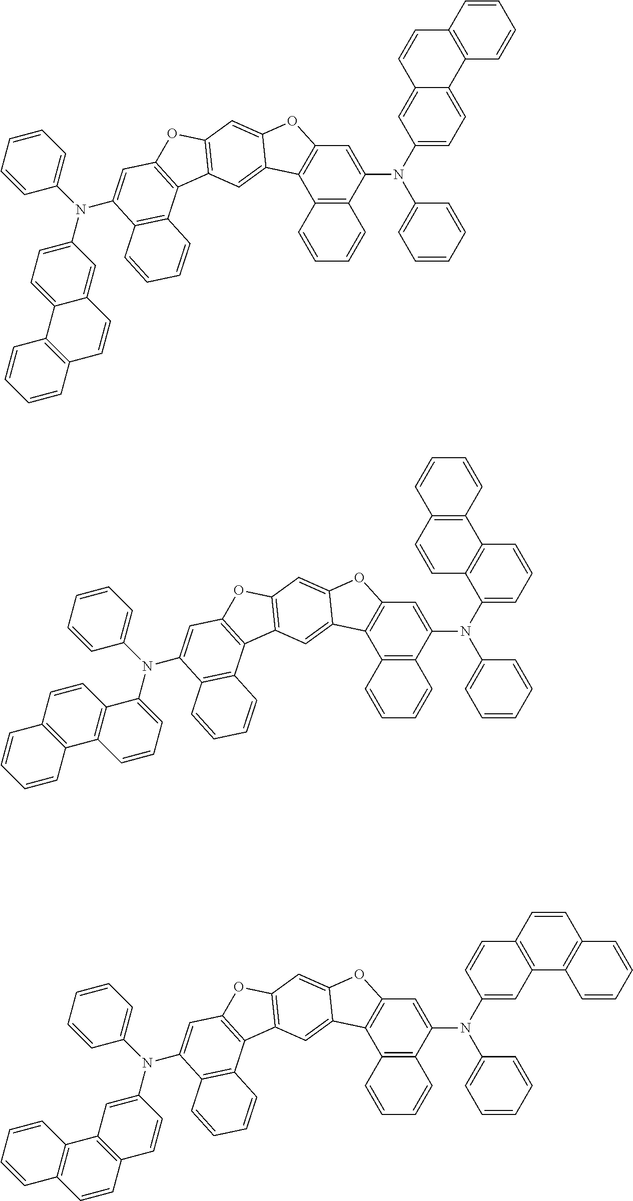 [SMILES: c1ccc(N(c2ccc3c(ccc4ccccc43)c2)c2cc3oc4cc5oc6cc(N(c7ccccc7)c7ccc8c(ccc9ccccc98)c7)c7ccccc7c6c5cc4c3c3ccccc23)cc1 ; c1ccc(N(c2cccc3c2ccc2ccccc23)c2cc3oc4cc5oc6cc(N(c7ccccc7)c7cccc8c7ccc7ccccc78)c7ccccc7c6c5cc4c3c3ccccc23)cc1 ; c1ccc(N(c2ccc3ccc4ccccc4c3c2)c2cc3oc4cc5oc6cc(N(c7ccccc7)c7ccc8ccc9ccccc9c8c7)c7ccccc7c6c5cc4c3c3ccccc23)cc1]

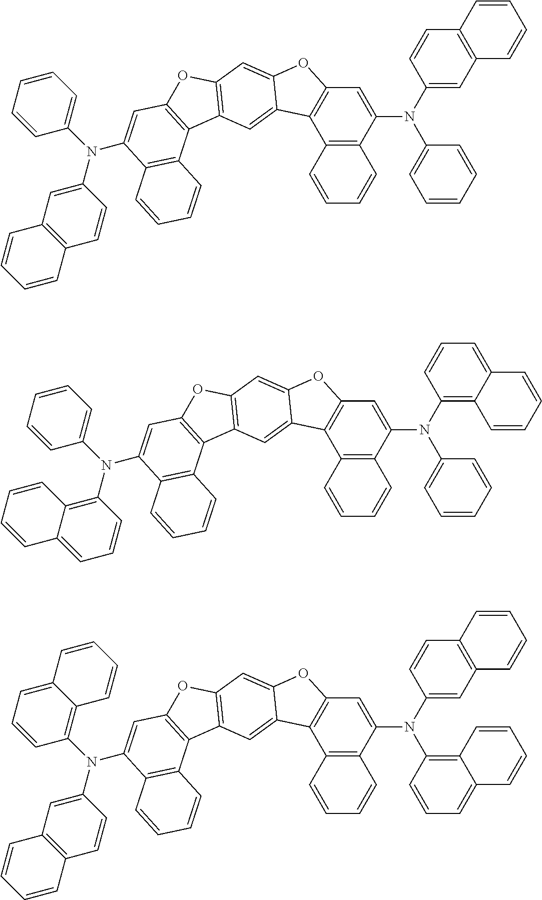 [SMILES: c1ccc(N(c2ccc3ccccc3c2)c2cc3oc4cc5oc6cc(N(c7ccccc7)c7ccc8ccccc8c7)c7ccccc7c6c5cc4c3c3ccccc23)cc1 ; c1ccc(N(c2cccc3ccccc23)c2cc3oc4cc5oc6cc(N(c7ccccc7)c7cccc8ccccc78)c7ccccc7c6c5cc4c3c3ccccc23)cc1 ; c1ccc2cc(N(c3cccc4ccccc34)c3cc4oc5cc6oc7cc(N(c8ccc9ccccc9c8)c8cccc9ccccc89)c8ccccc8c7c6cc5c4c4ccccc34)ccc2c1]

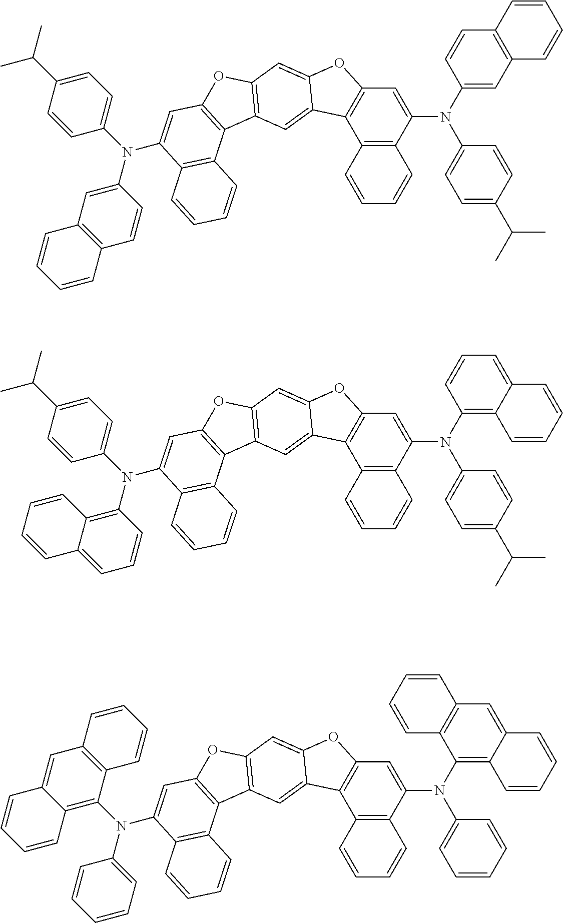 [SMILES: CC(C)c1ccc(N(c2ccc3ccccc3c2)c2cc3oc4cc5oc6cc(N(c7ccc(C(C)C)cc7)c7ccc8ccccc8c7)c7ccccc7c6c5cc4c3c3ccccc23)cc1 ; CC(C)c1ccc(N(c2cccc3ccccc23)c2cc3oc4cc5oc6cc(N(c7ccc(C(C)C)cc7)c7cccc8ccccc78)c7ccccc7c6c5cc4c3c3ccccc23)cc1 ; c1ccc(N(c2c3ccccc3cc3ccccc23)c2cc3oc4cc5oc6cc(N(c7ccccc7)c7c8ccccc8cc8ccccc78)c7ccccc7c6c5cc4c3c3ccccc23)cc1]

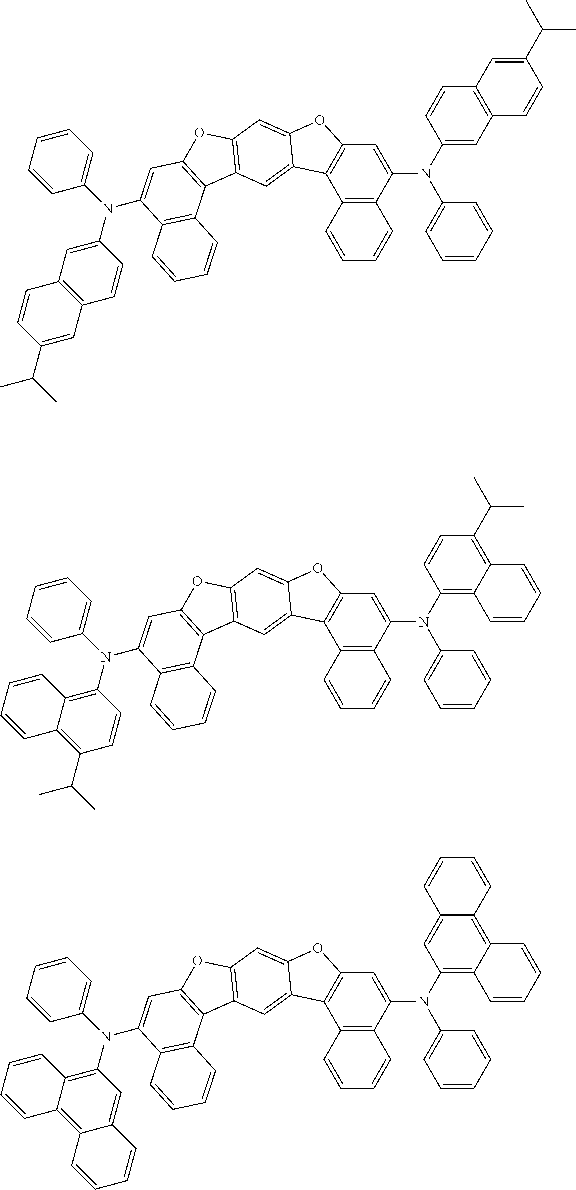 [SMILES: CC(C)c1ccc2cc(N(c3ccccc3)c3cc4oc5cc6oc7cc(N(c8ccccc8)c8ccc9cc(C(C)C)ccc9c8)c8ccccc8c7c6cc5c4c4ccccc34)ccc2c1 ; CC(C)c1ccc(N(c2ccccc2)c2cc3oc4cc5oc6cc(N(c7ccccc7)c7ccc(C(C)C)c8ccccc78)c7ccccc7c6c5cc4c3c3ccccc23)c2ccccc12 ; c1ccc(N(c2cc3ccccc3c3ccccc23)c2cc3oc4cc5oc6cc(N(c7ccccc7)c7cc8ccccc8c8ccccc78)c7ccccc7c6c5cc4c3c3ccccc23)cc1]

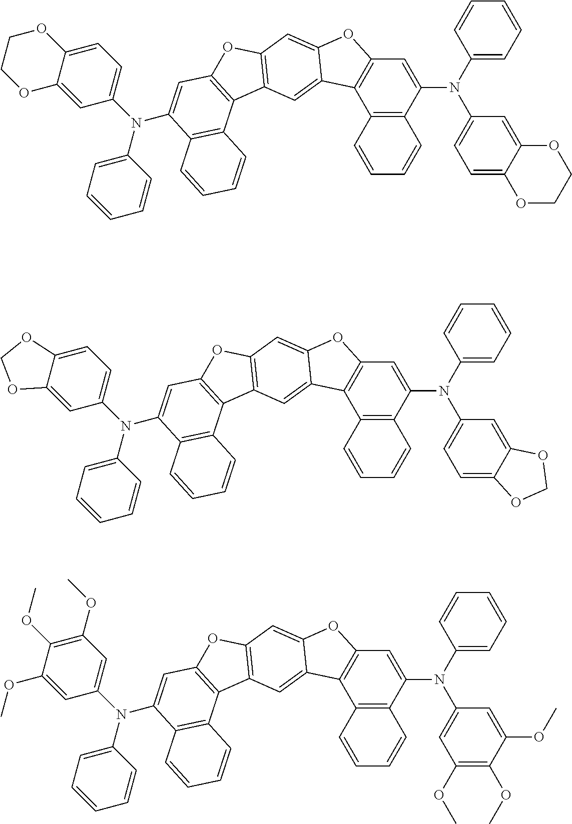

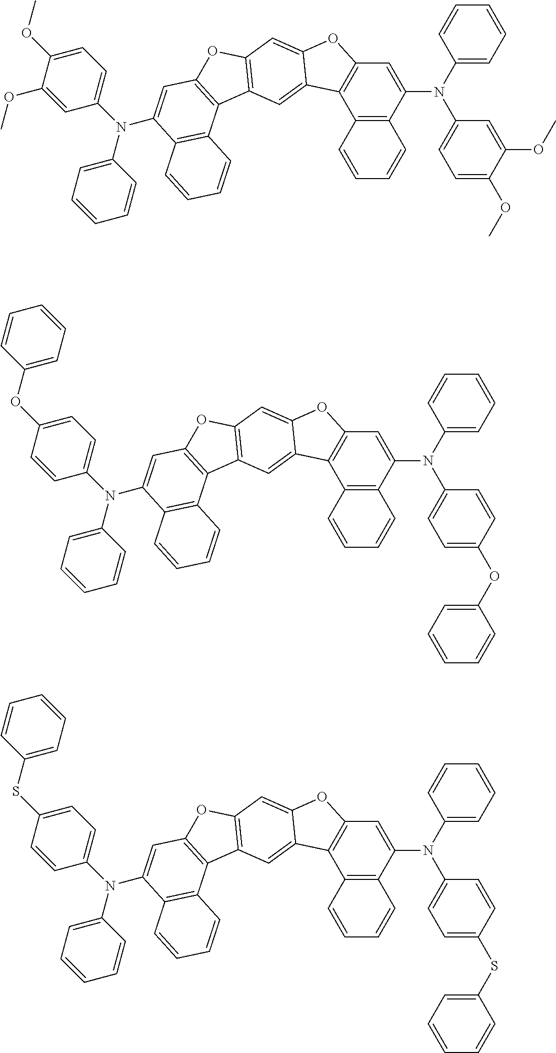

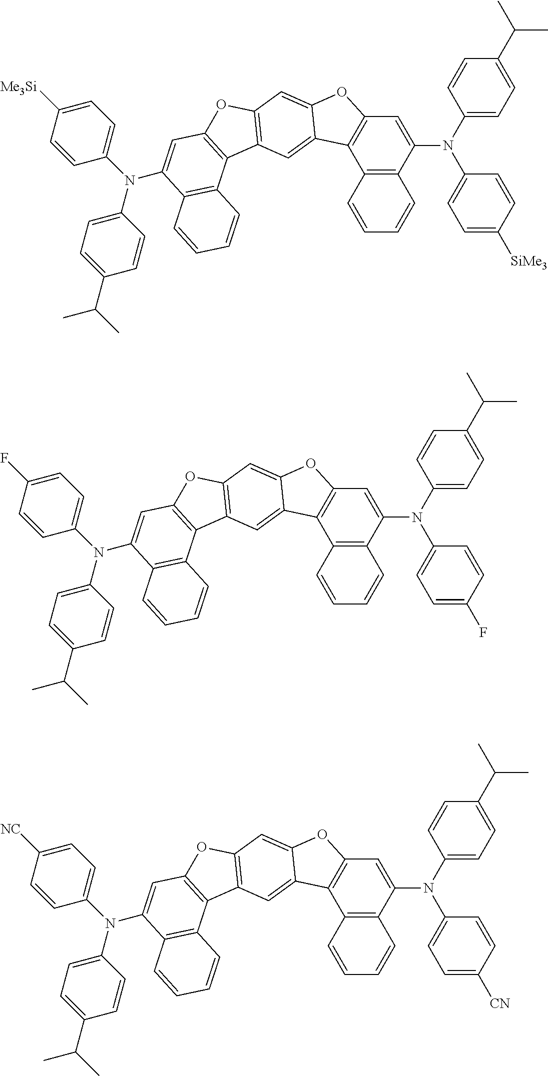 [SMILES: CC(C)c1ccc(N(c2ccc([Si](C)(C)C)cc2)c2cc3oc4cc5oc6cc(N(c7ccc(C(C)C)cc7)c7ccc([Si](C)(C)C)cc7)c7ccccc7c6c5cc4c3c3ccccc23)cc1 ; CC(C)c1ccc(N(c2ccc(F)cc2)c2cc3oc4cc5oc6cc(N(c7ccc(F)cc7)c7ccc(C(C)C)cc7)c7ccccc7c6c5cc4c3c3ccccc23)cc1 ; CC(C)c1ccc(N(c2ccc(C#N)cc2)c2cc3oc4cc5oc6cc(N(c7ccc(C#N)cc7)c7ccc(C(C)C)cc7)c7ccccc7c6c5cc4c3c3ccccc23)cc1]

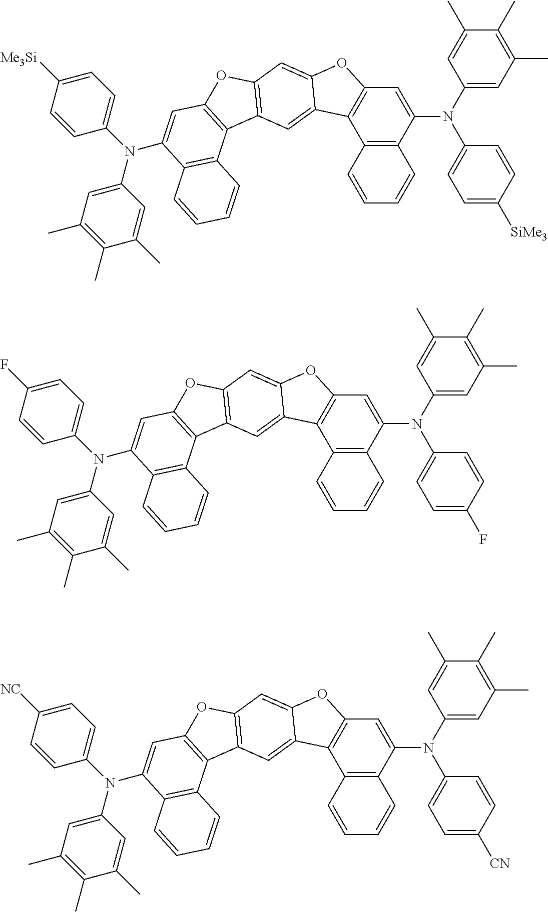 [SMILES: Cc1cc(N(c2ccc([Si](C)(C)C)cc2)c2cc3oc4cc5oc6cc(N(c7ccc([Si](C)(C)C)cc7)c7cc(C)c(C)c(C)c7)c7ccccc7c6c5cc4c3c3ccccc23)cc(C)c1C ; Cc1cc(N(c2ccc(F)cc2)c2cc3oc4cc5oc6cc(N(c7ccc(F)cc7)c7cc(C)c(C)c(C)c7)c7ccccc7c6c5cc4c3c3ccccc23)cc(C)c1C ; Cc1cc(N(c2ccc(C#N)cc2)c2cc3oc4cc5oc6cc(N(c7ccc(C#N)cc7)c7cc(C)c(C)c(C)c7)c7ccccc7c6c5cc4c3c3ccccc23)cc(C)c1C]

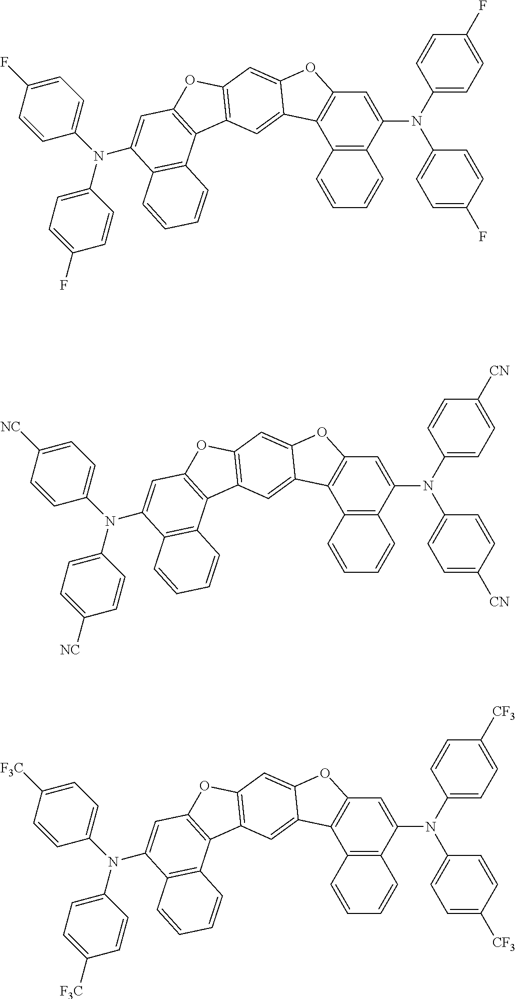 [SMILES: Fc1ccc(N(c2ccc(F)cc2)c2cc3oc4cc5oc6cc(N(c7ccc(F)cc7)c7ccc(F)cc7)c7ccccc7c6c5cc4c3c3ccccc23)cc1 ; N#Cc1ccc(N(c2ccc(C#N)cc2)c2cc3oc4cc5oc6cc(N(c7ccc(C#N)cc7)c7ccc(C#N)cc7)c7ccccc7c6c5cc4c3c3ccccc23)cc1 ; FC(F)(F)c1ccc(N(c2ccc(C(F)(F)F)cc2)c2cc3oc4cc5oc6cc(N(c7ccc(C(F)(F)F)cc7)c7ccc(C(F)(F)F)cc7)c7ccccc7c6c5cc4c3c3ccccc23)cc1]

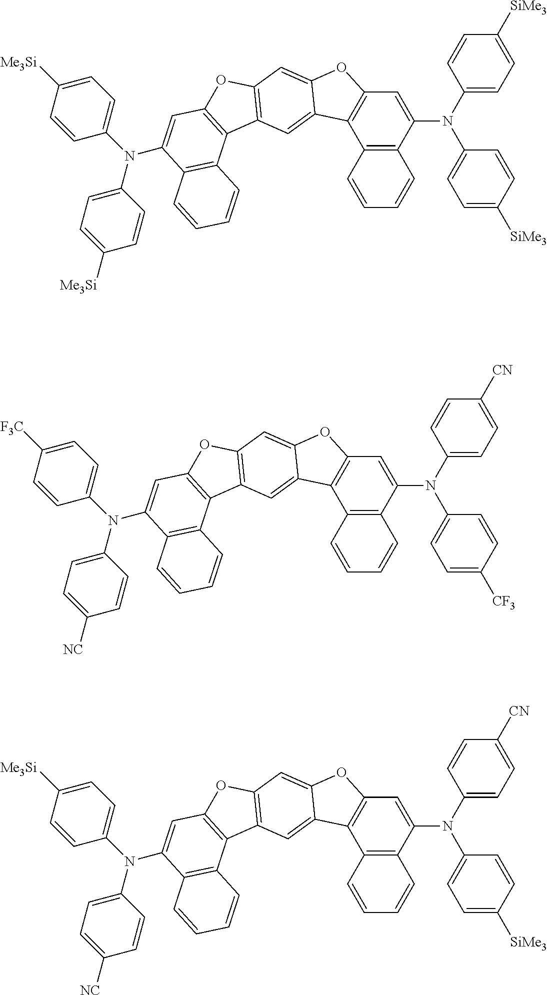 [SMILES: C[Si](C)(C)c1ccc(N(c2ccc([Si](C)(C)C)cc2)c2cc3oc4cc5oc6cc(N(c7ccc([Si](C)(C)C)cc7)c7ccc([Si](C)(C)C)cc7)c7ccccc7c6c5cc4c3c3ccccc23)cc1 ; N#Cc1ccc(N(c2ccc(C(F)(F)F)cc2)c2cc3oc4cc5oc6cc(N(c7ccc(C#N)cc7)c7ccc(C(F)(F)F)cc7)c7ccccc7c6c5cc4c3c3ccccc23)cc1 ; C[Si](C)(C)c1ccc(N(c2ccc(C#N)cc2)c2cc3oc4cc5oc6cc(N(c7ccc(C#N)cc7)c7ccc([Si](C)(C)C)cc7)c7ccccc7c6c5cc4c3c3ccccc23)cc1]

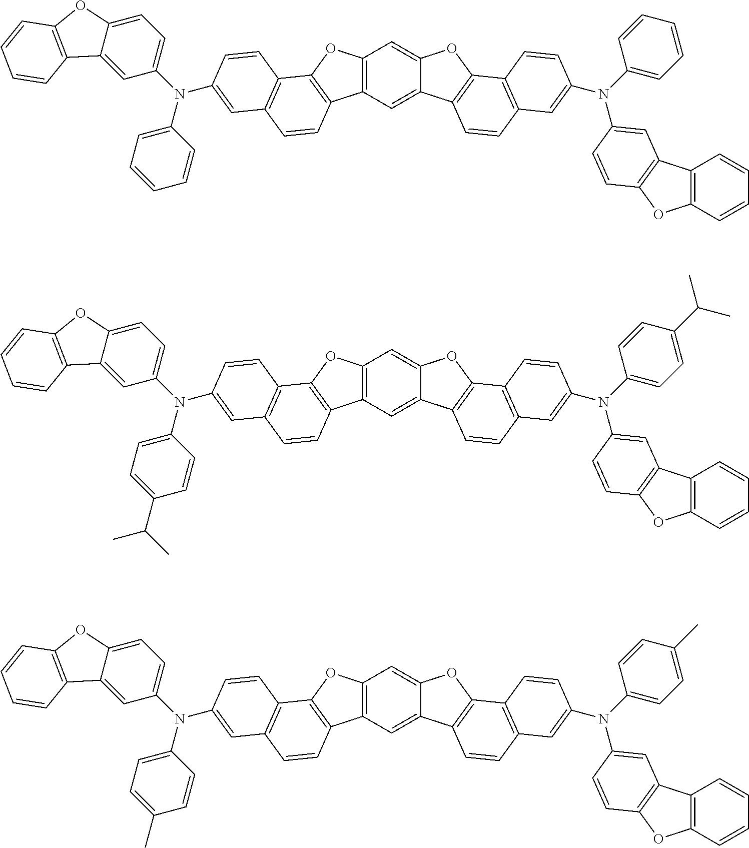 [SMILES: c1ccc(N(c2ccc3c(ccc4c5cc6c(cc5oc34)oc3c4ccc(N(c5ccccc5)c5ccc7oc8ccccc8c7c5)cc4ccc63)c2)c2ccc3oc4ccccc4c3c2)cc1 ; CC(C)c1ccc(N(c2ccc3c(ccc4c5cc6c(cc5oc34)oc3c4ccc(N(c5ccc(C(C)C)cc5)c5ccc7oc8ccccc8c7c5)cc4ccc63)c2)c2ccc3oc4ccccc4c3c2)cc1 ; Cc1ccc(N(c2ccc3c(ccc4c5cc6c(cc5oc34)oc3c4ccc(N(c5ccc(C)cc5)c5ccc7oc8ccccc8c7c5)cc4ccc63)c2)c2ccc3oc4ccccc4c3c2)cc1]

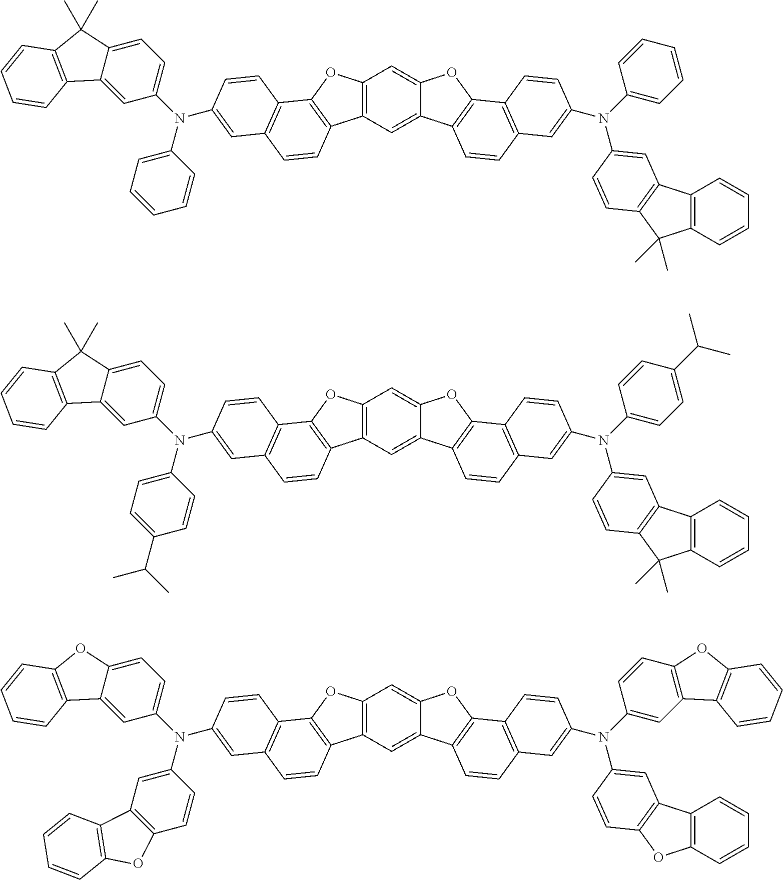 [SMILES: CC1(C)c2ccccc2-c2cc(N(c3ccccc3)c3ccc4c(ccc5c6cc7c(cc6oc45)oc4c5ccc(N(c6ccccc6)c6ccc8c(c6)-c6ccccc6C8(C)C)cc5ccc74)c3)ccc21 ; CC(C)c1ccc(N(c2ccc3c(c2)-c2ccccc2C3(C)C)c2ccc3c(ccc4c5cc6c(cc5oc34)oc3c4ccc(N(c5ccc(C(C)C)cc5)c5ccc7c(c5)-c5ccccc5C7(C)C)cc4ccc63)c2)cc1 ; c1ccc2c(c1)oc1ccc(N(c3ccc4c(ccc5c6cc7c(cc6oc45)oc4c5ccc(N(c6ccc8oc9ccccc9c8c6)c6ccc8oc9ccccc9c8c6)cc5ccc74)c3)c3ccc4oc5ccccc5c4c3)cc12]

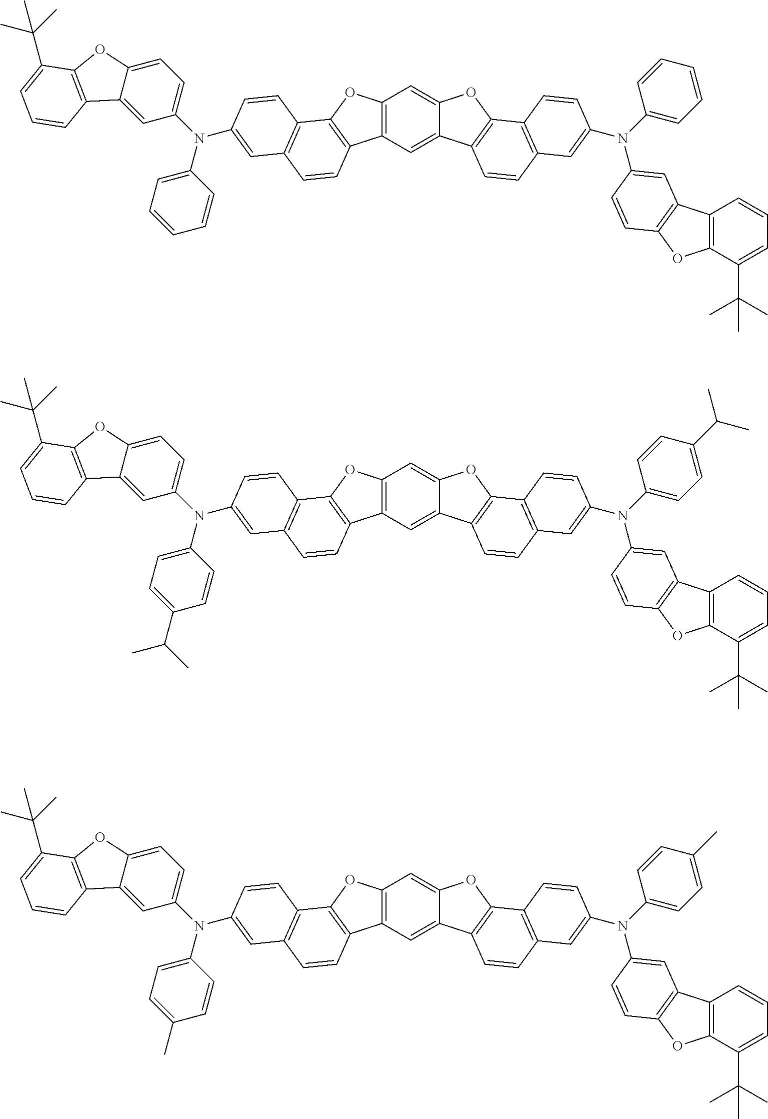

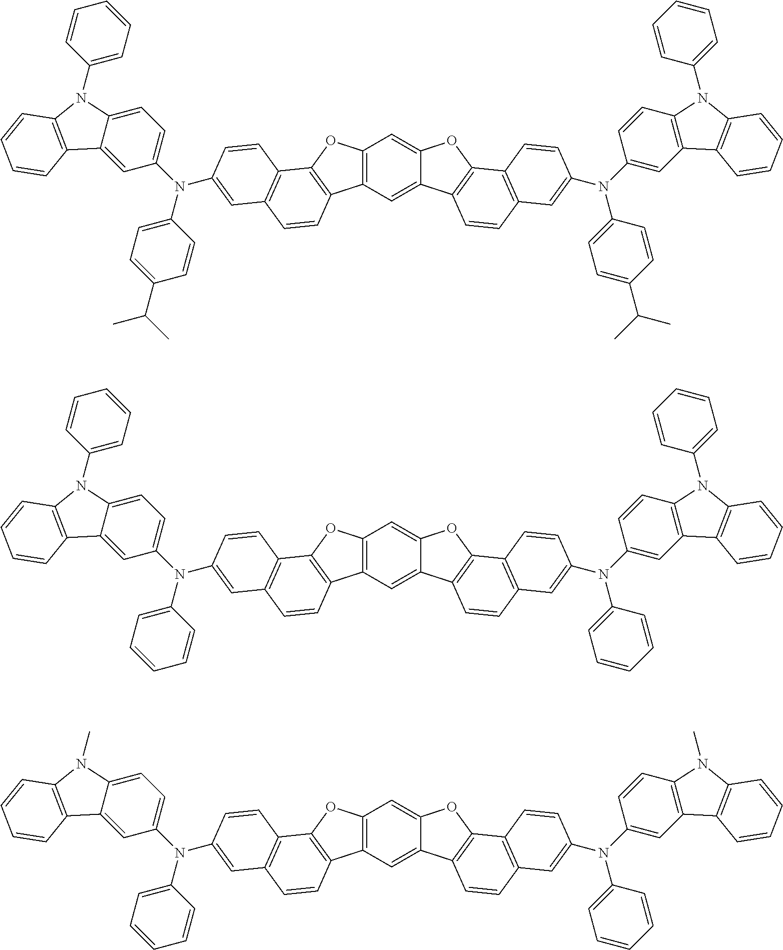 [SMILES: CC(C)c1ccc(N(c2ccc3c(ccc4c5cc6c(cc5oc34)oc3c4ccc(N(c5ccc(C(C)C)cc5)c5ccc7c(c5)c5ccccc5n7-c5ccccc5)cc4ccc63)c2)c2ccc3c(c2)c2ccccc2n3-c2ccccc2)cc1 ; c1ccc(N(c2ccc3c(ccc4c5cc6c(cc5oc34)oc3c4ccc(N(c5ccccc5)c5ccc7c(c5)c5ccccc5n7-c5ccccc5)cc4ccc63)c2)c2ccc3c(c2)c2ccccc2n3-c2ccccc2)cc1 ; Cn1c2ccccc2c2cc(N(c3ccccc3)c3ccc4c(ccc5c6cc7c(cc6oc45)oc4c5ccc(N(c6ccccc6)c6ccc8c(c6)c6ccccc6n8C)cc5ccc74)c3)ccc21]

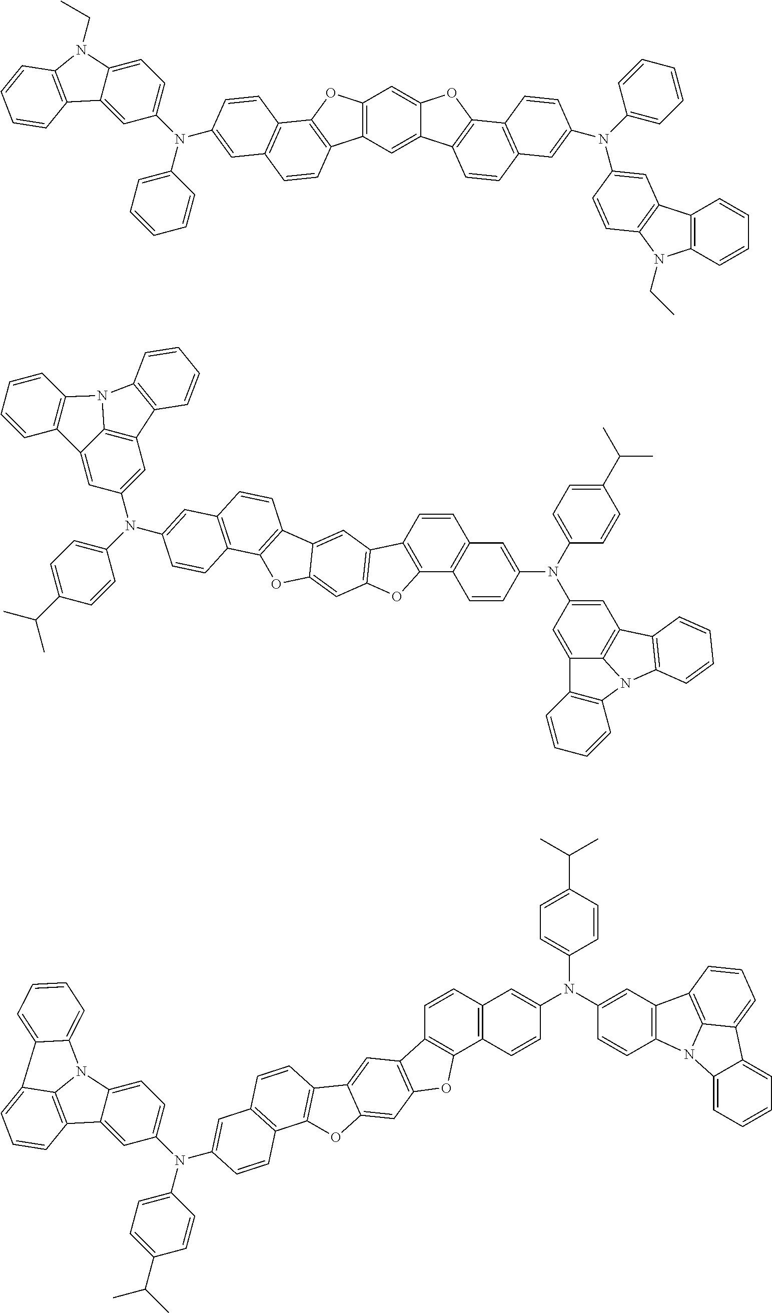 [SMILES: CCn1c2ccccc2c2cc(N(c3ccccc3)c3ccc4c(ccc5c6cc7c(cc6oc45)oc4c5ccc(N(c6ccccc6)c6ccc8c(c6)c6ccccc6n8CC)cc5ccc74)c3)ccc21 ; CC(C)c1ccc(N(c2ccc3c(ccc4c5cc6c(cc5oc34)oc3c4ccc(N(c5ccc(C(C)C)cc5)c5cc7c8ccccc8n8c9ccccc9c(c5)c78)cc4ccc63)c2)c2cc3c4ccccc4n4c5ccccc5c(c2)c34)cc1 ; CC(C)c1ccc(N(c2ccc3c(ccc4c5cc6c(cc5oc34)oc3c4ccc(N(c5ccc(C(C)C)cc5)c5ccc7c(c5)c5cccc8c9ccccc9n7c85)cc4ccc63)c2)c2ccc3c(c2)c2cccc4c5ccccc5n3c42)cc1]

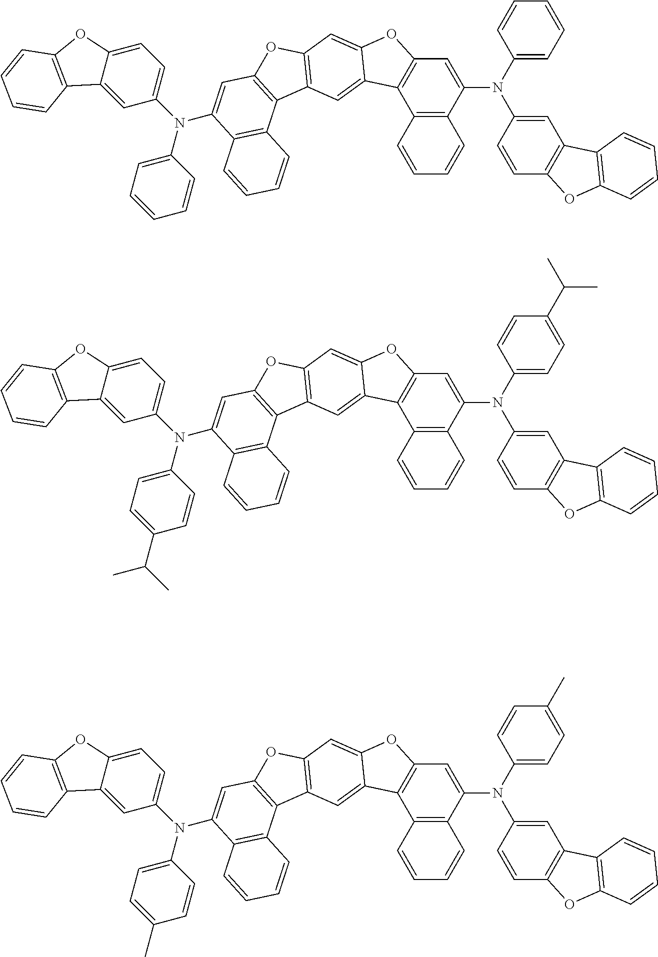 [SMILES: c1ccc(N(c2ccc3oc4ccccc4c3c2)c2cc3oc4cc5oc6cc(N(c7ccccc7)c7ccc8oc9ccccc9c8c7)c7ccccc7c6c5cc4c3c3ccccc23)cc1 ; CC(C)c1ccc(N(c2ccc3oc4ccccc4c3c2)c2cc3oc4cc5oc6cc(N(c7ccc(C(C)C)cc7)c7ccc8oc9ccccc9c8c7)c7ccccc7c6c5cc4c3c3ccccc23)cc1 ; Cc1ccc(N(c2ccc3oc4ccccc4c3c2)c2cc3oc4cc5oc6cc(N(c7ccc(C)cc7)c7ccc8oc9ccccc9c8c7)c7ccccc7c6c5cc4c3c3ccccc23)cc1]

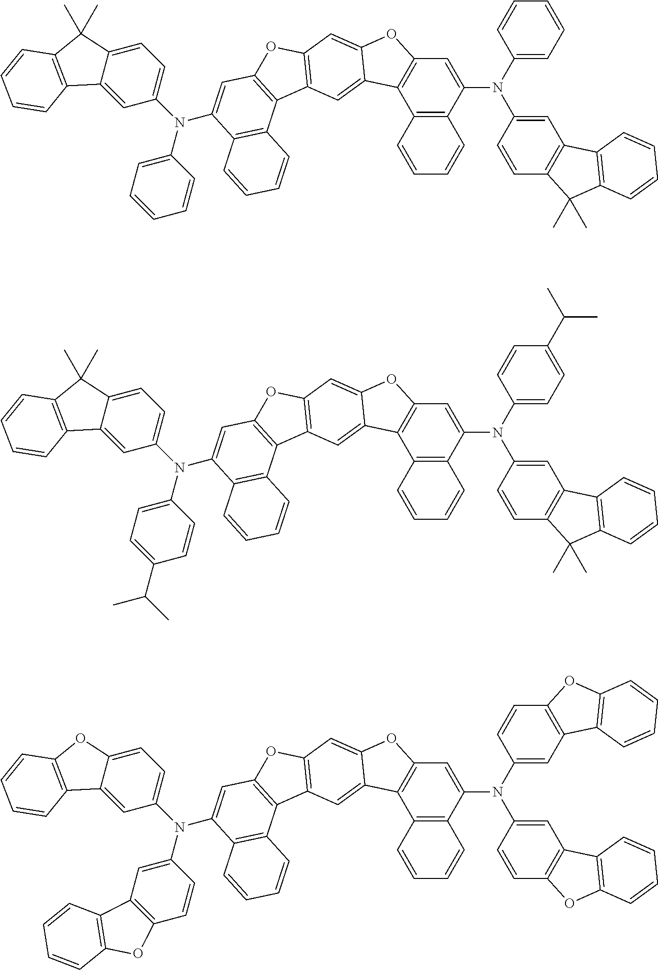 [SMILES: CC1(C)c2ccccc2-c2cc(N(c3ccccc3)c3cc4oc5cc6oc7cc(N(c8ccccc8)c8ccc9c(c8)-c8ccccc8C9(C)C)c8ccccc8c7c6cc5c4c4ccccc34)ccc21 ; CC(C)c1ccc(N(c2ccc3c(c2)-c2ccccc2C3(C)C)c2cc3oc4cc5oc6cc(N(c7ccc(C(C)C)cc7)c7ccc8c(c7)-c7ccccc7C8(C)C)c7ccccc7c6c5cc4c3c3ccccc23)cc1 ; c1ccc2c(c1)oc1ccc(N(c3ccc4oc5ccccc5c4c3)c3cc4oc5cc6oc7cc(N(c8ccc9oc%10ccccc%10c9c8)c8ccc9oc%10ccccc%10c9c8)c8ccccc8c7c6cc5c4c4ccccc34)cc12]

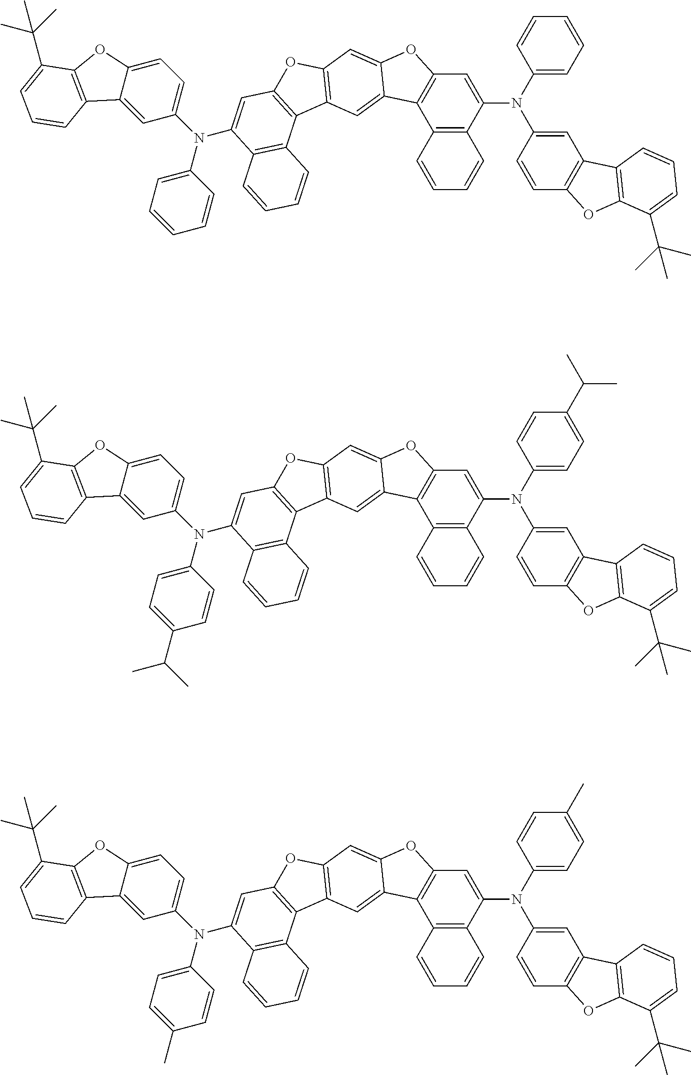

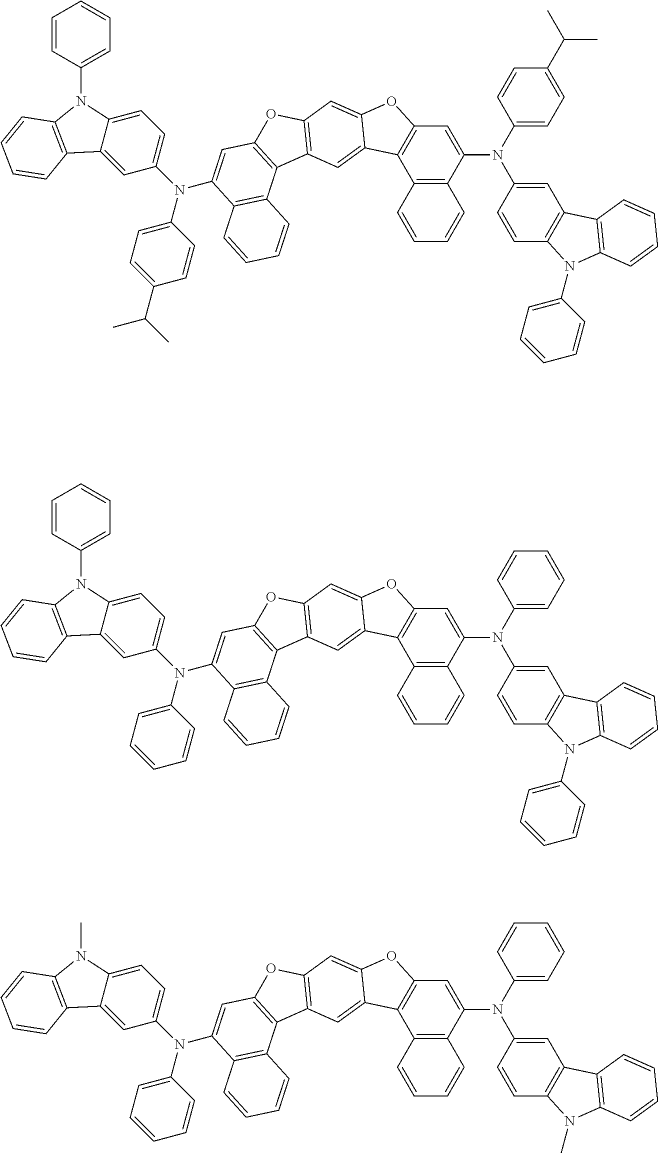 [SMILES: CC(C)c1ccc(N(c2ccc3c(c2)c2ccccc2n3-c2ccccc2)c2cc3oc4cc5oc6cc(N(c7ccc(C(C)C)cc7)c7ccc8c(c7)c7ccccc7n8-c7ccccc7)c7ccccc7c6c5cc4c3c3ccccc23)cc1 ; c1ccc(N(c2ccc3c(c2)c2ccccc2n3-c2ccccc2)c2cc3oc4cc5oc6cc(N(c7ccccc7)c7ccc8c(c7)c7ccccc7n8-c7ccccc7)c7ccccc7c6c5cc4c3c3ccccc23)cc1 ; Cn1c2ccccc2c2cc(N(c3ccccc3)c3cc4oc5cc6oc7cc(N(c8ccccc8)c8ccc9c(c8)c8ccccc8n9C)c8ccccc8c7c6cc5c4c4ccccc34)ccc21]

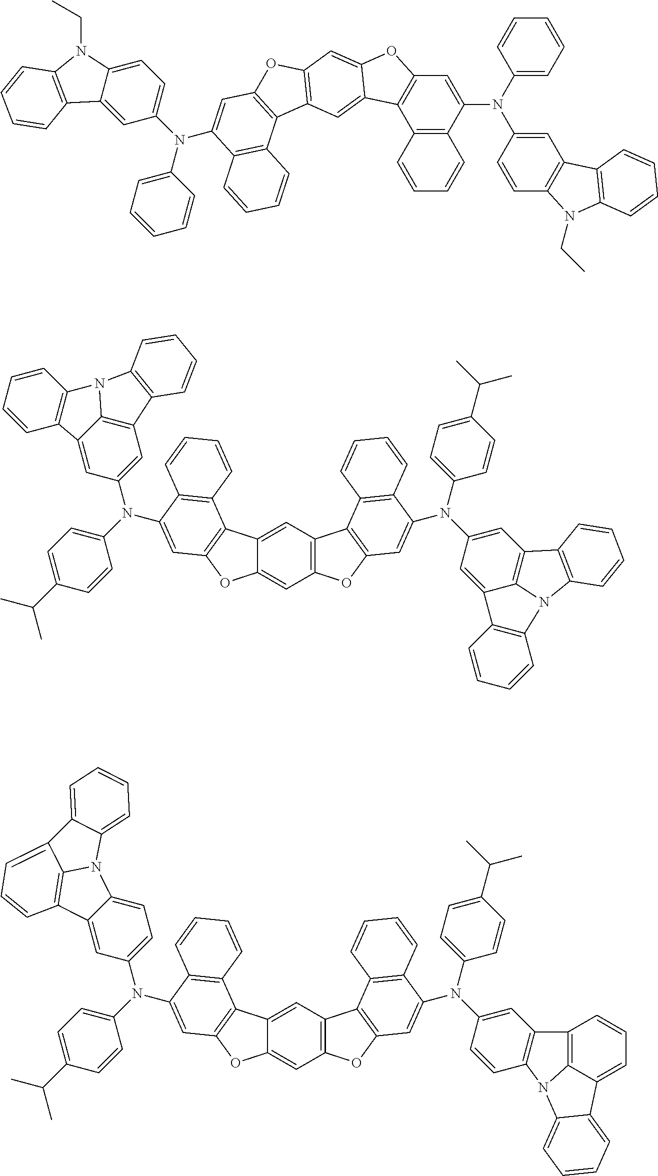 [SMILES: CCn1c2ccccc2c2cc(N(c3ccccc3)c3cc4oc5cc6oc7cc(N(c8ccccc8)c8ccc9c(c8)c8ccccc8n9CC)c8ccccc8c7c6cc5c4c4ccccc34)ccc21 ; CC(C)c1ccc(N(c2cc3c4ccccc4n4c5ccccc5c(c2)c34)c2cc3oc4cc5oc6cc(N(c7ccc(C(C)C)cc7)c7cc8c9ccccc9n9c%10ccccc%10c(c7)c89)c7ccccc7c6c5cc4c3c3ccccc23)cc1 ; CC(C)c1ccc(N(c2ccc3c(c2)c2cccc4c5ccccc5n3c42)c2cc3oc4cc5oc6cc(N(c7ccc(C(C)C)cc7)c7ccc8c(c7)c7cccc9c%10ccccc%10n8c97)c7ccccc7c6c5cc4c3c3ccccc23)cc1]

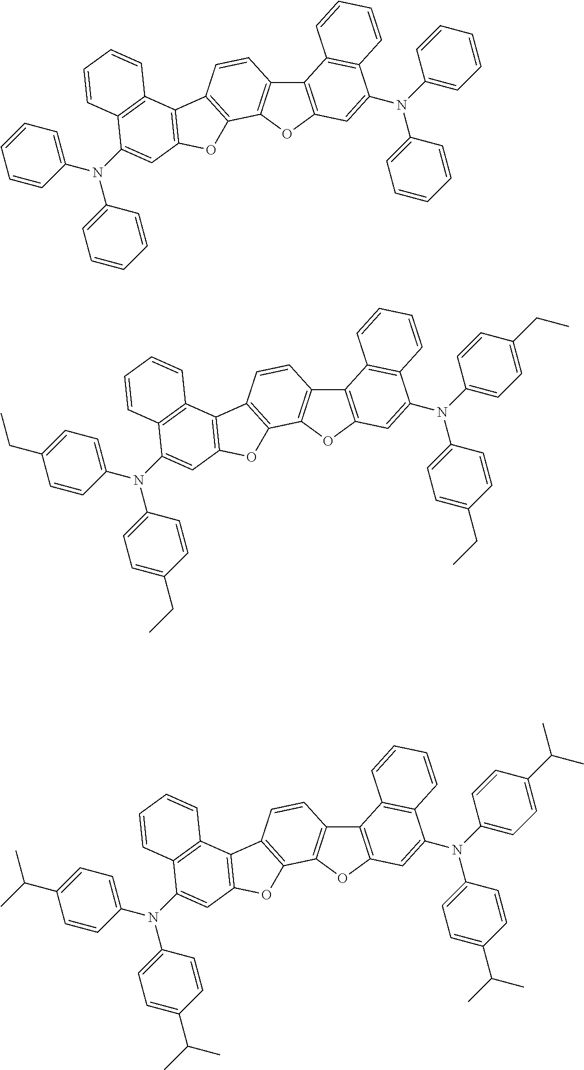

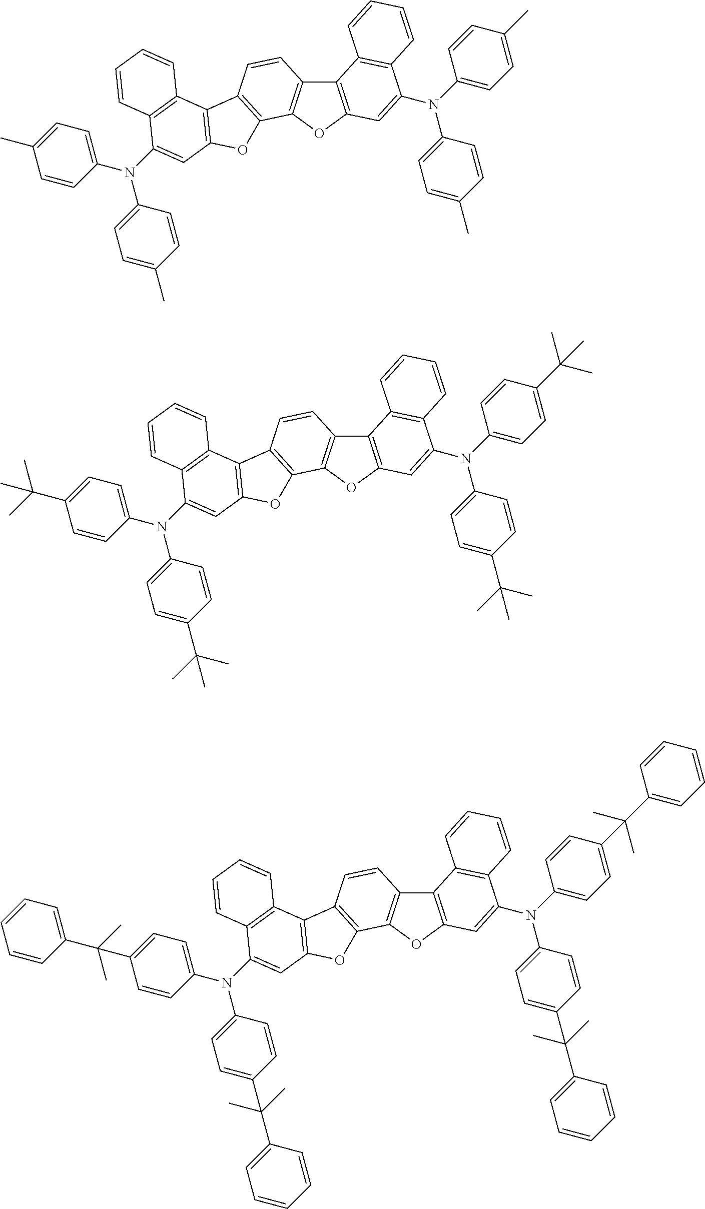

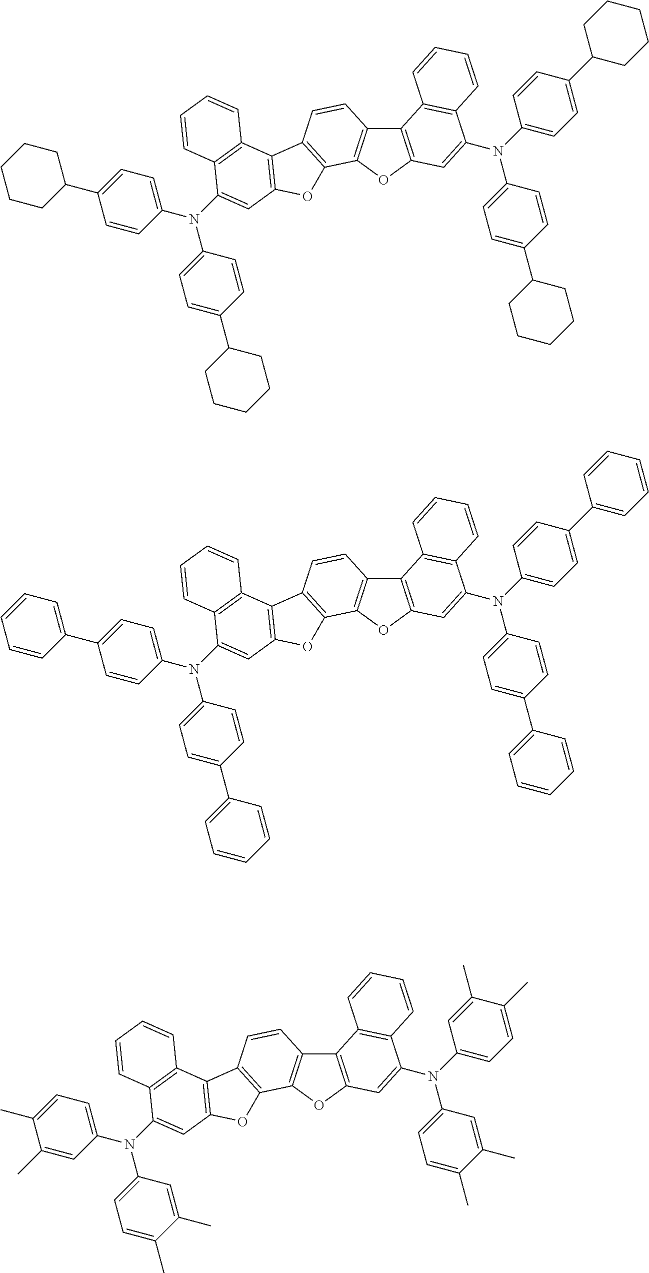

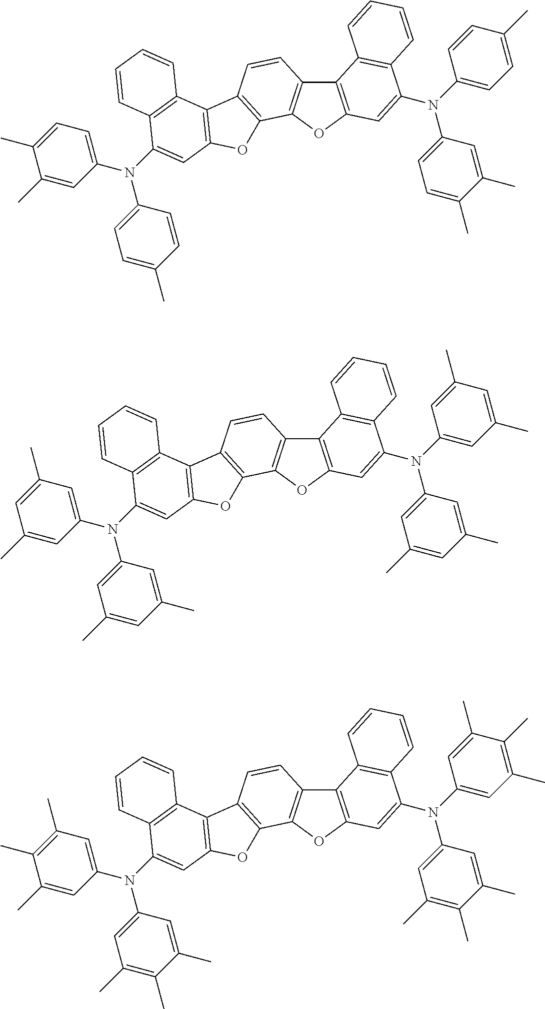

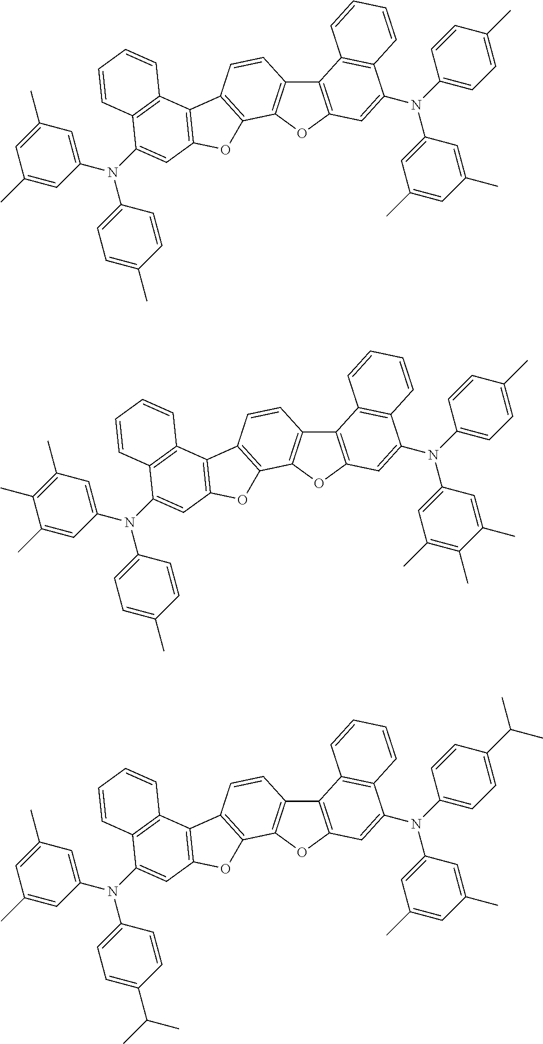

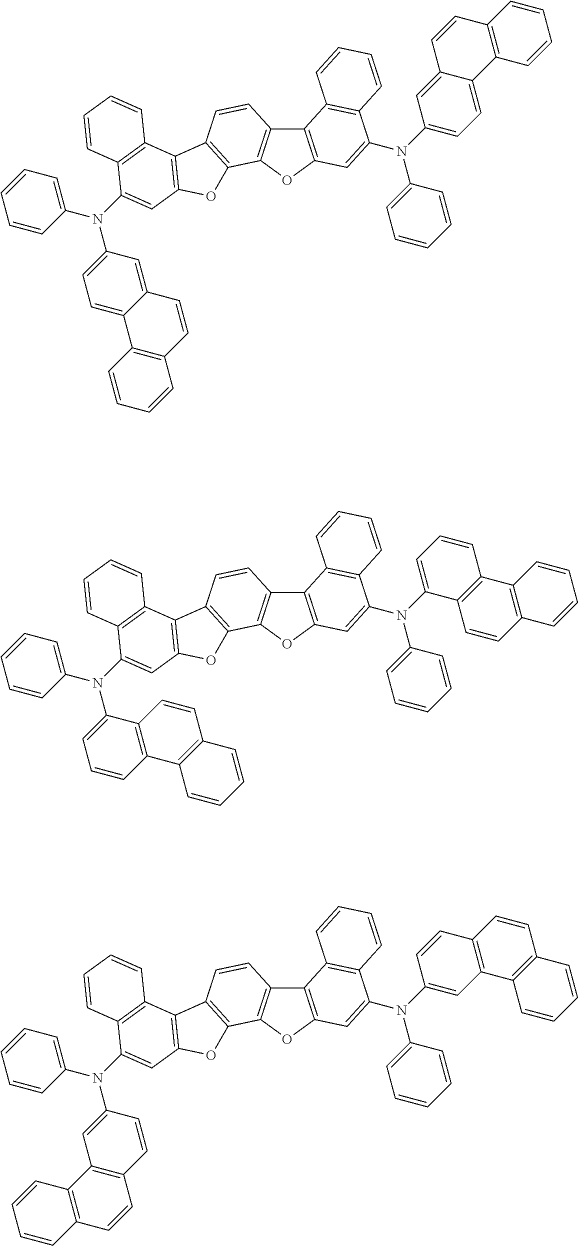 [SMILES: c1ccc(N(c2ccc3c(ccc4ccccc43)c2)c2cc3oc4c(ccc5c4oc4cc(N(c6ccccc6)c6ccc7c(ccc8ccccc87)c6)c6ccccc6c45)c3c3ccccc23)cc1 ; c1ccc(N(c2cccc3c2ccc2ccccc23)c2cc3oc4c(ccc5c4oc4cc(N(c6ccccc6)c6cccc7c6ccc6ccccc67)c6ccccc6c45)c3c3ccccc23)cc1 ; c1ccc(N(c2ccc3ccc4ccccc4c3c2)c2cc3oc4c(ccc5c4oc4cc(N(c6ccccc6)c6ccc7ccc8ccccc8c7c6)c6ccccc6c45)c3c3ccccc23)cc1]

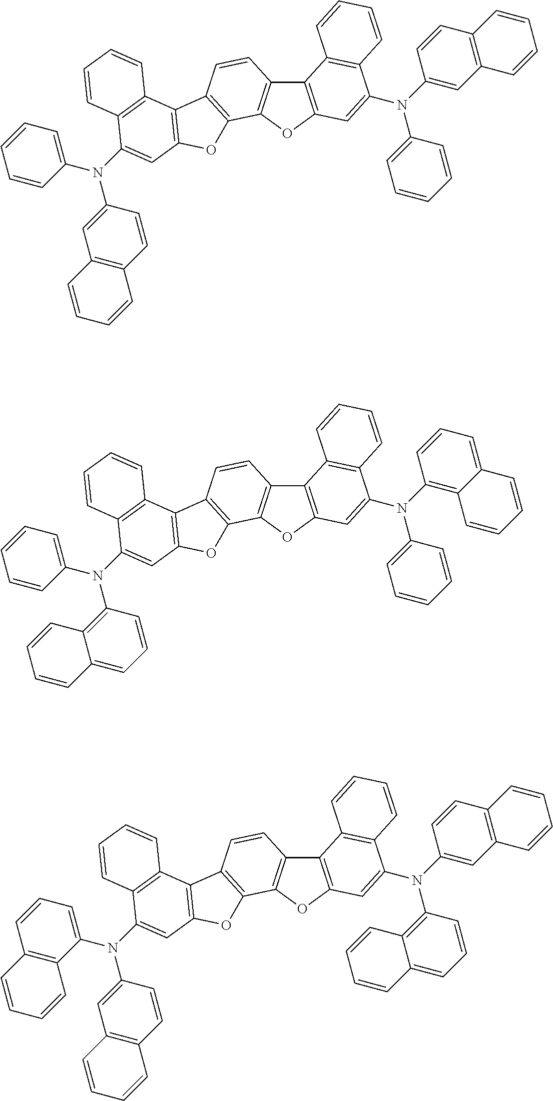 [SMILES: c1ccc(N(c2ccc3ccccc3c2)c2cc3oc4c(ccc5c4oc4cc(N(c6ccccc6)c6ccc7ccccc7c6)c6ccccc6c45)c3c3ccccc23)cc1 ; c1ccc(N(c2cccc3ccccc23)c2cc3oc4c(ccc5c4oc4cc(N(c6ccccc6)c6cccc7ccccc67)c6ccccc6c45)c3c3ccccc23)cc1 ; c1ccc2cc(N(c3cccc4ccccc34)c3cc4oc5c(ccc6c5oc5cc(N(c7ccc8ccccc8c7)c7cccc8ccccc78)c7ccccc7c56)c4c4ccccc34)ccc2c1]

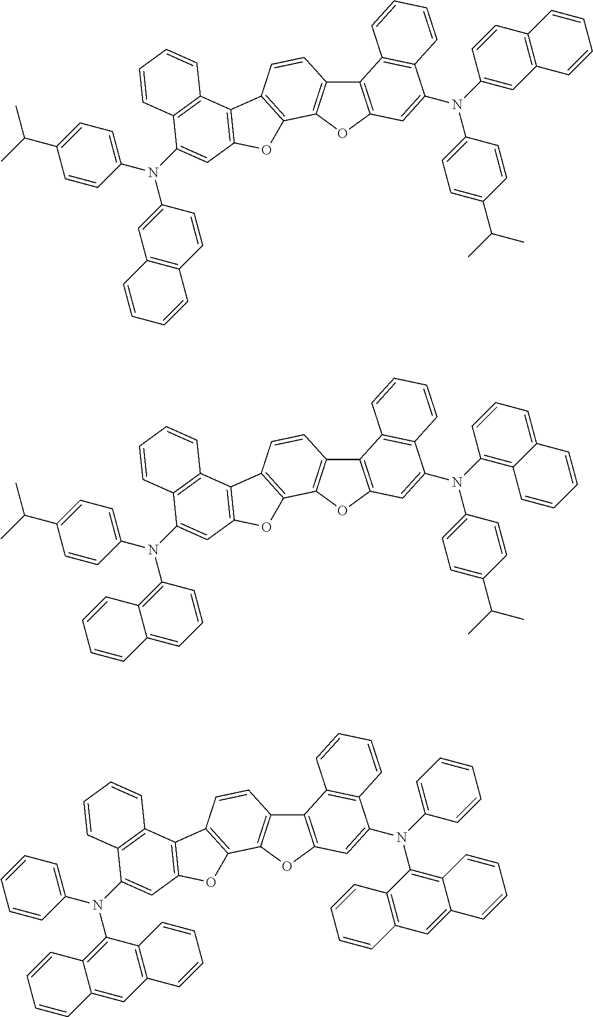 [SMILES: CC(C)c1ccc(N(c2ccc3ccccc3c2)c2cc3oc4c(ccc5c4oc4cc(N(c6ccc(C(C)C)cc6)c6ccc7ccccc7c6)c6ccccc6c45)c3c3ccccc23)cc1 ; CC(C)c1ccc(N(c2cccc3ccccc23)c2cc3oc4c(ccc5c4oc4cc(N(c6ccc(C(C)C)cc6)c6cccc7ccccc67)c6ccccc6c45)c3c3ccccc23)cc1 ; c1ccc(N(c2c3ccccc3cc3ccccc23)c2cc3oc4c(ccc5c4oc4cc(N(c6ccccc6)c6c7ccccc7cc7ccccc67)c6ccccc6c45)c3c3ccccc23)cc1]

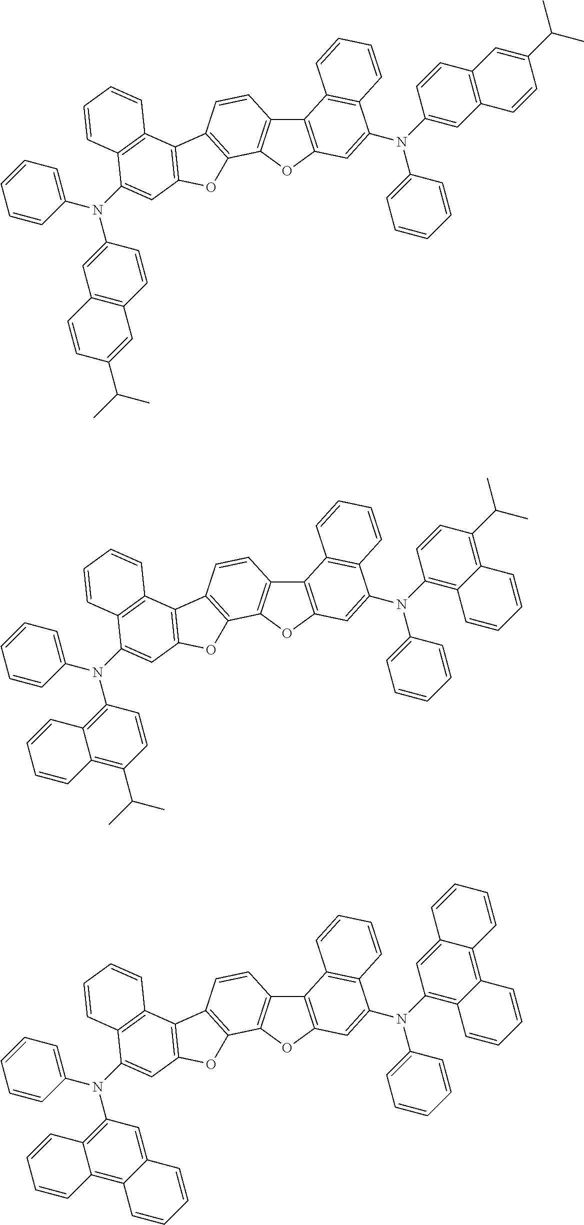 [SMILES: CC(C)c1ccc2cc(N(c3ccccc3)c3cc4oc5c(ccc6c5oc5cc(N(c7ccccc7)c7ccc8cc(C(C)C)ccc8c7)c7ccccc7c56)c4c4ccccc34)ccc2c1 ; CC(C)c1ccc(N(c2ccccc2)c2cc3oc4c(ccc5c4oc4cc(N(c6ccccc6)c6ccc(C(C)C)c7ccccc67)c6ccccc6c45)c3c3ccccc23)c2ccccc12 ; c1ccc(N(c2cc3ccccc3c3ccccc23)c2cc3oc4c(ccc5c4oc4cc(N(c6ccccc6)c6cc7ccccc7c7ccccc67)c6ccccc6c45)c3c3ccccc23)cc1]

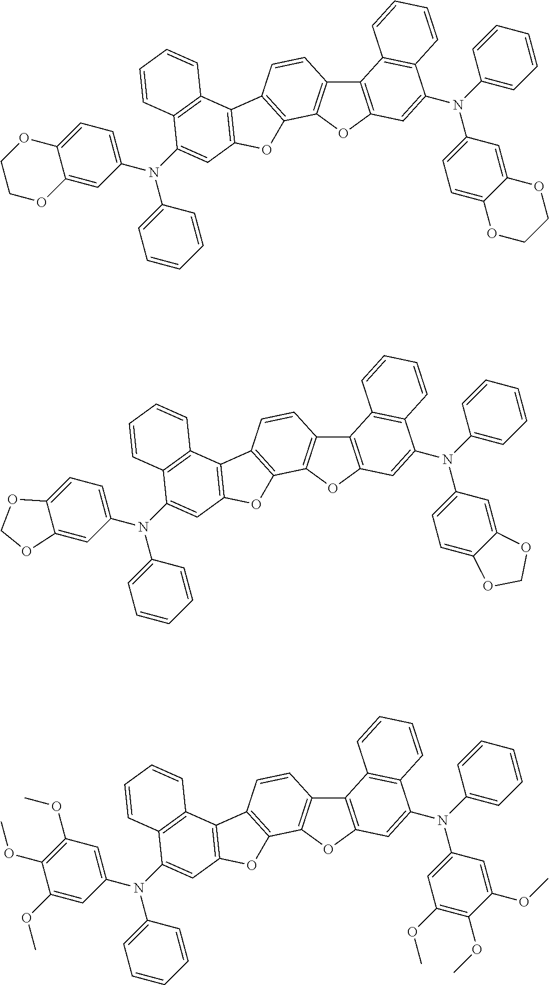

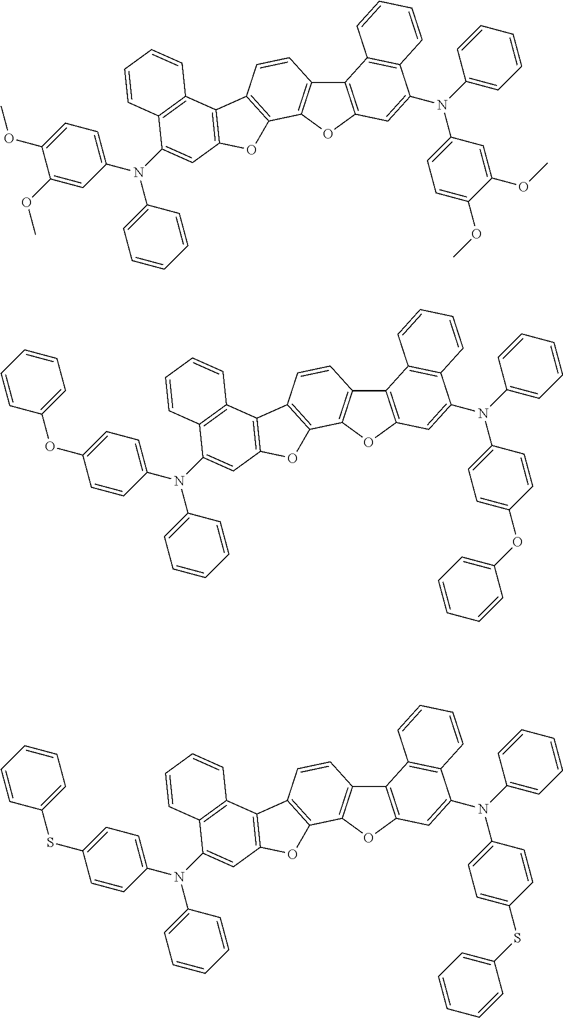

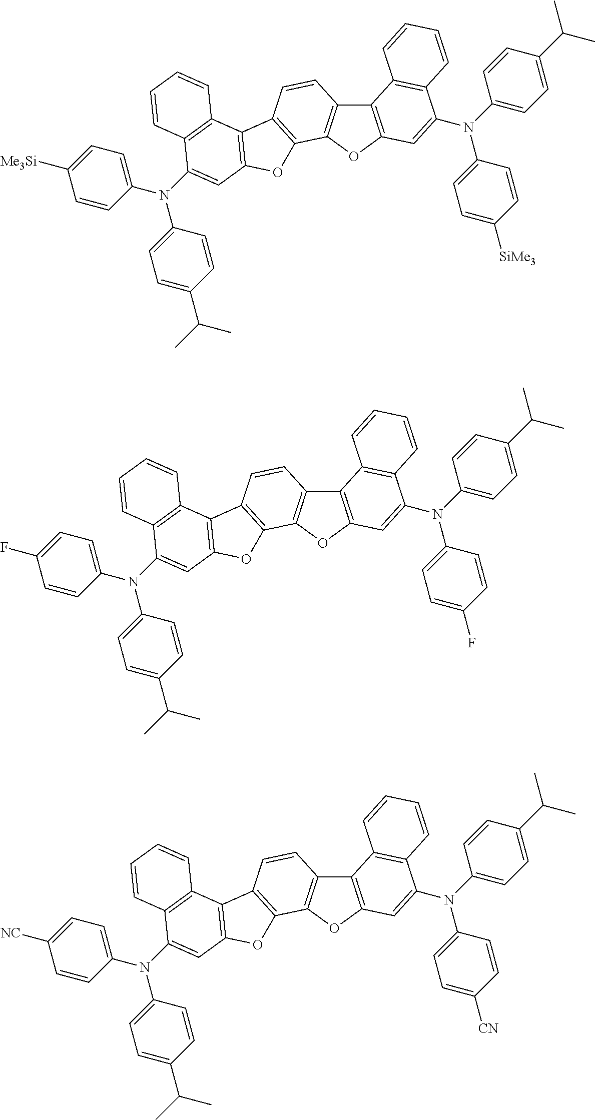 [SMILES: CC(C)c1ccc(N(c2ccc([Si](C)(C)C)cc2)c2cc3oc4c(ccc5c4oc4cc(N(c6ccc(C(C)C)cc6)c6ccc([Si](C)(C)C)cc6)c6ccccc6c45)c3c3ccccc23)cc1 ; CC(C)c1ccc(N(c2ccc(F)cc2)c2cc3oc4c(ccc5c4oc4cc(N(c6ccc(F)cc6)c6ccc(C(C)C)cc6)c6ccccc6c45)c3c3ccccc23)cc1 ; CC(C)c1ccc(N(c2ccc(C#N)cc2)c2cc3oc4c(ccc5c4oc4cc(N(c6ccc(C#N)cc6)c6ccc(C(C)C)cc6)c6ccccc6c45)c3c3ccccc23)cc1]

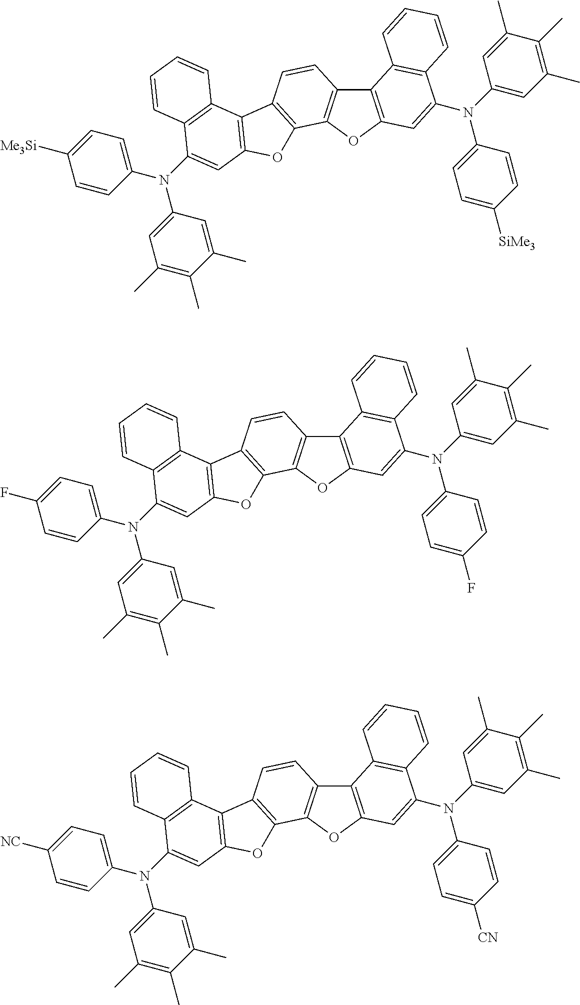 [SMILES: Cc1cc(N(c2ccc([Si](C)(C)C)cc2)c2cc3oc4c(ccc5c4oc4cc(N(c6ccc([Si](C)(C)C)cc6)c6cc(C)c(C)c(C)c6)c6ccccc6c45)c3c3ccccc23)cc(C)c1C ; Cc1cc(N(c2ccc(F)cc2)c2cc3oc4c(ccc5c4oc4cc(N(c6ccc(F)cc6)c6cc(C)c(C)c(C)c6)c6ccccc6c45)c3c3ccccc23)cc(C)c1C ; Cc1cc(N(c2ccc(C#N)cc2)c2cc3oc4c(ccc5c4oc4cc(N(c6ccc(C#N)cc6)c6cc(C)c(C)c(C)c6)c6ccccc6c45)c3c3ccccc23)cc(C)c1C]

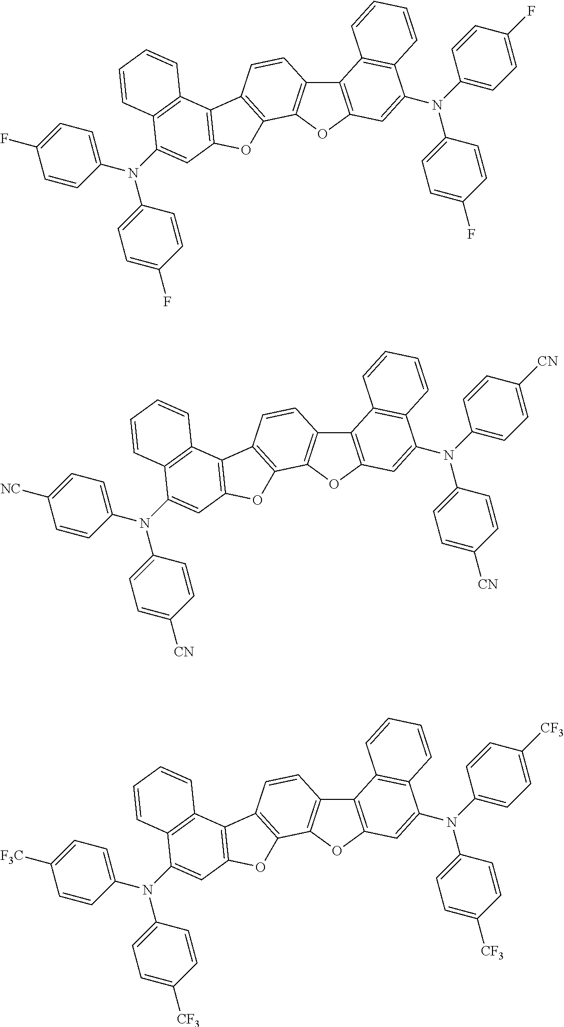 [SMILES: Fc1ccc(N(c2ccc(F)cc2)c2cc3oc4c(ccc5c4oc4cc(N(c6ccc(F)cc6)c6ccc(F)cc6)c6ccccc6c45)c3c3ccccc23)cc1 ; N#Cc1ccc(N(c2ccc(C#N)cc2)c2cc3oc4c(ccc5c4oc4cc(N(c6ccc(C#N)cc6)c6ccc(C#N)cc6)c6ccccc6c45)c3c3ccccc23)cc1 ; FC(F)(F)c1ccc(N(c2ccc(C(F)(F)F)cc2)c2cc3oc4c(ccc5c4oc4cc(N(c6ccc(C(F)(F)F)cc6)c6ccc(C(F)(F)F)cc6)c6ccccc6c45)c3c3ccccc23)cc1]

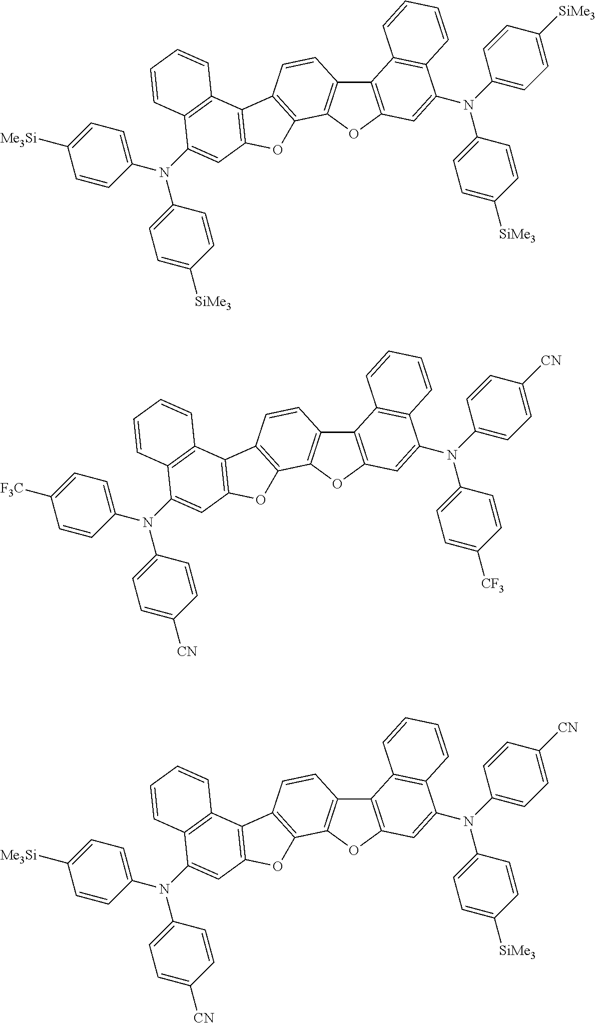 [SMILES: C[Si](C)(C)c1ccc(N(c2ccc([Si](C)(C)C)cc2)c2cc3oc4c(ccc5c4oc4cc(N(c6ccc([Si](C)(C)C)cc6)c6ccc([Si](C)(C)C)cc6)c6ccccc6c45)c3c3ccccc23)cc1 ; N#Cc1ccc(N(c2ccc(C(F)(F)F)cc2)c2cc3oc4c(ccc5c4oc4cc(N(c6ccc(C#N)cc6)c6ccc(C(F)(F)F)cc6)c6ccccc6c45)c3c3ccccc23)cc1 ; C[Si](C)(C)c1ccc(N(c2ccc(C#N)cc2)c2cc3oc4c(ccc5c4oc4cc(N(c6ccc(C#N)cc6)c6ccc([Si](C)(C)C)cc6)c6ccccc6c45)c3c3ccccc23)cc1]

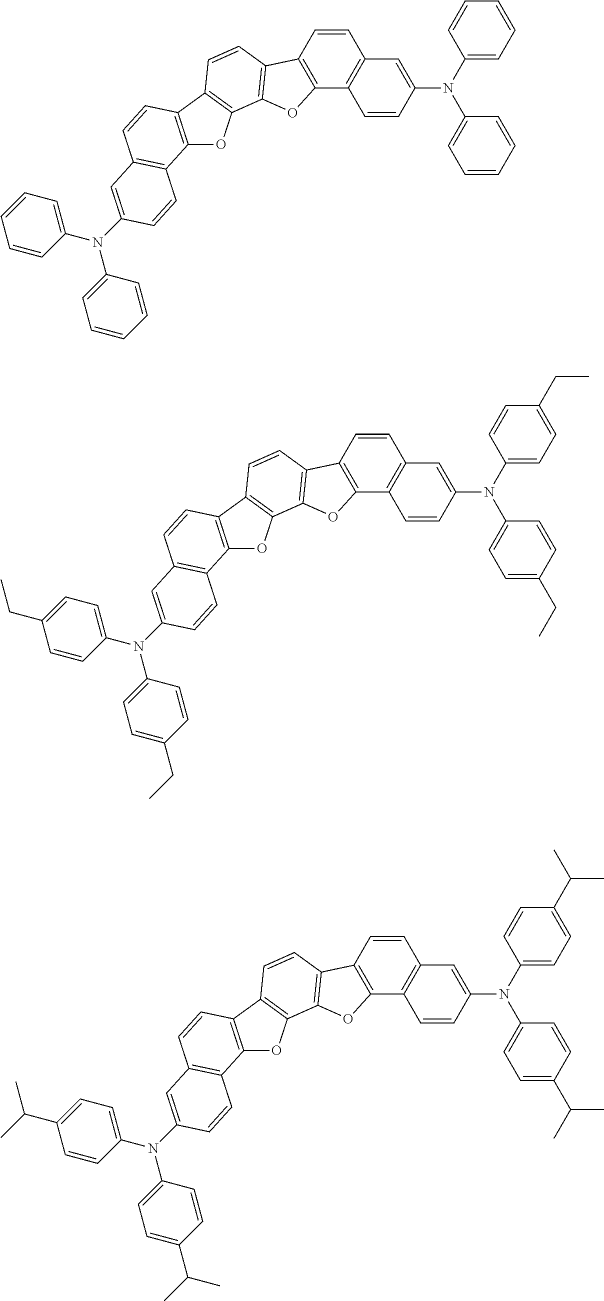

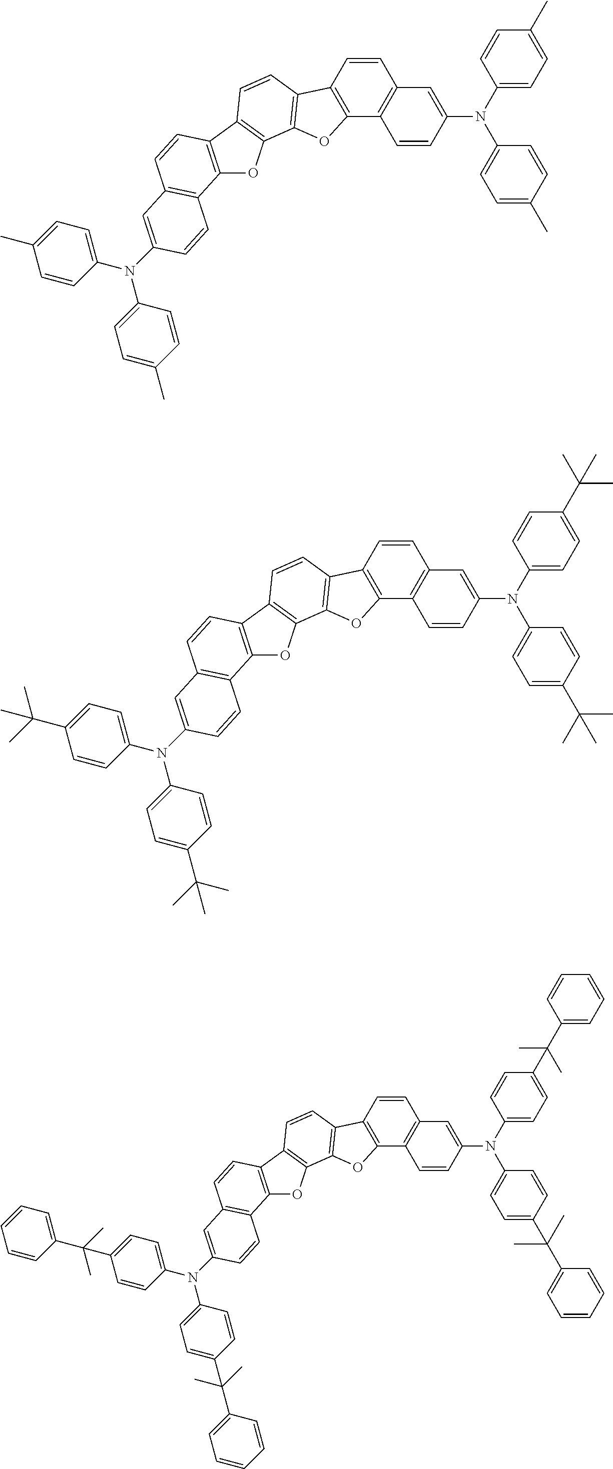

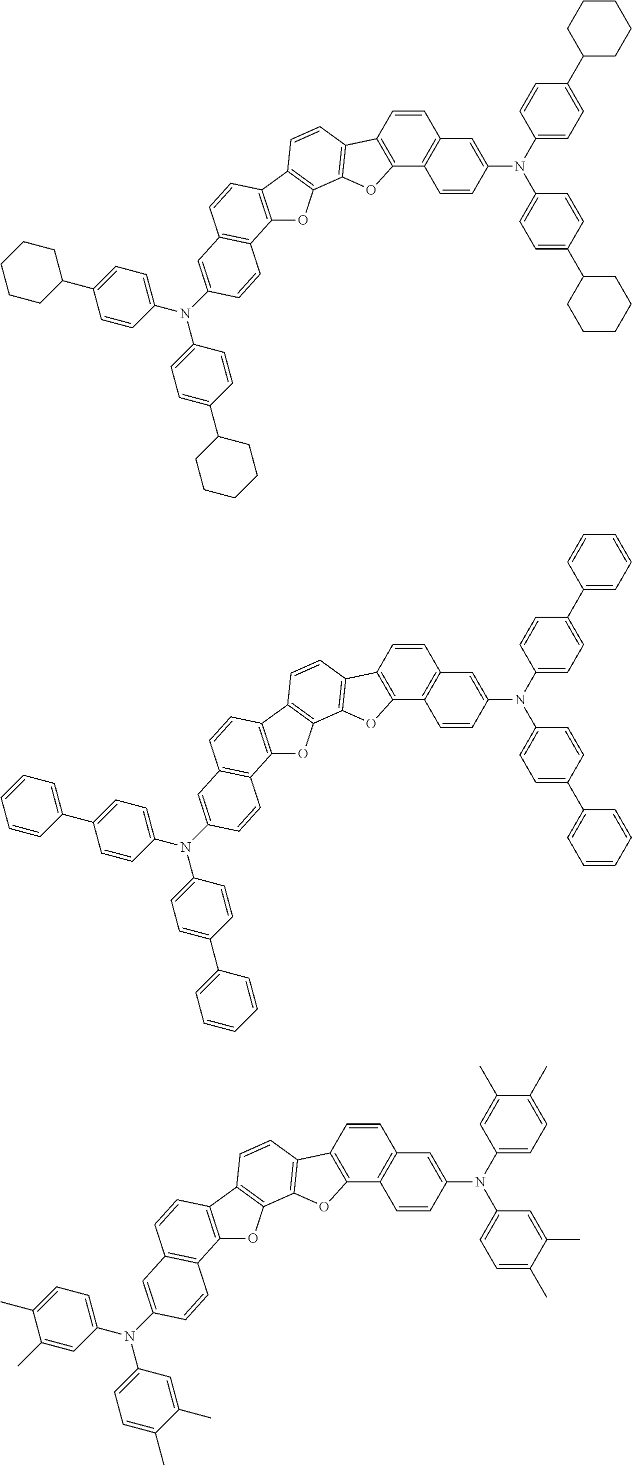

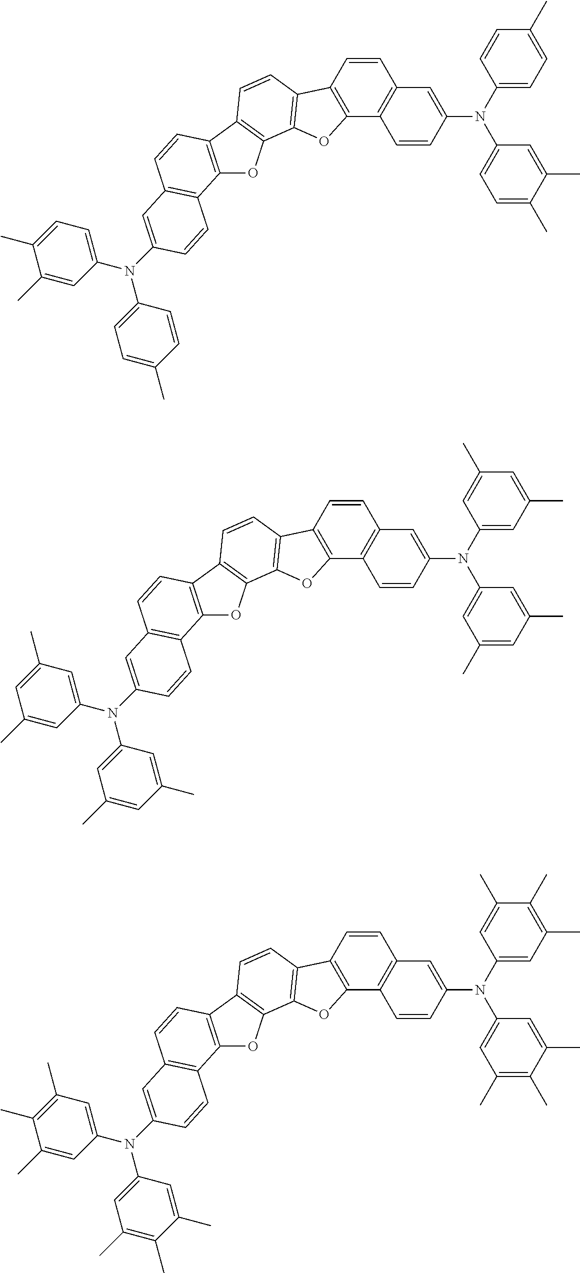

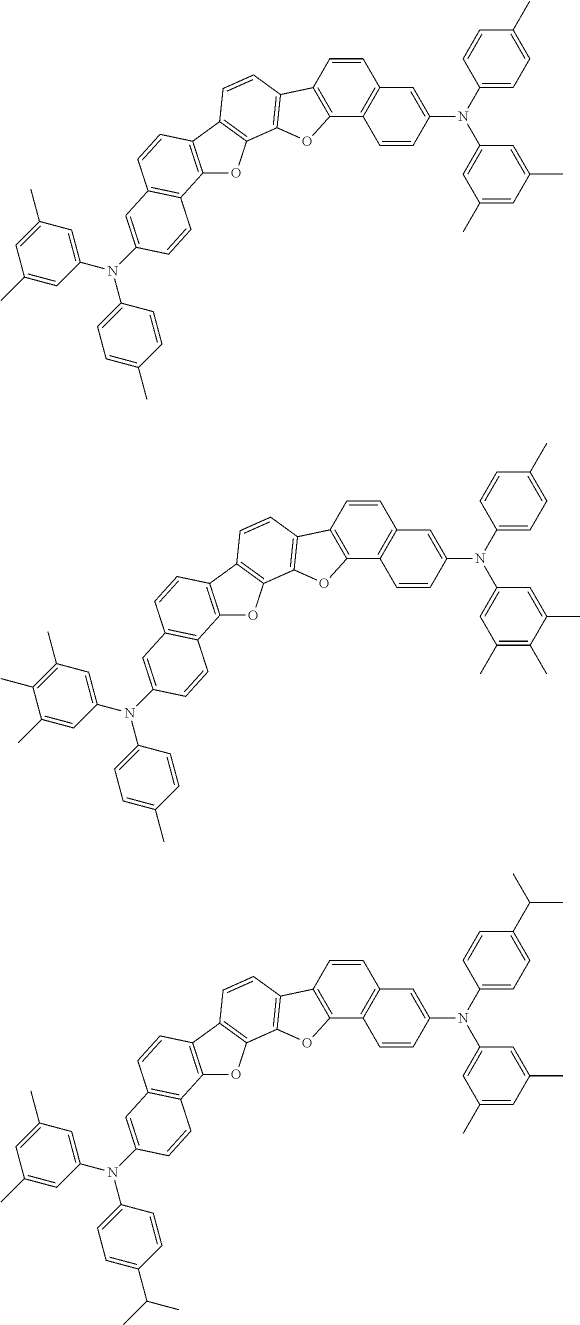

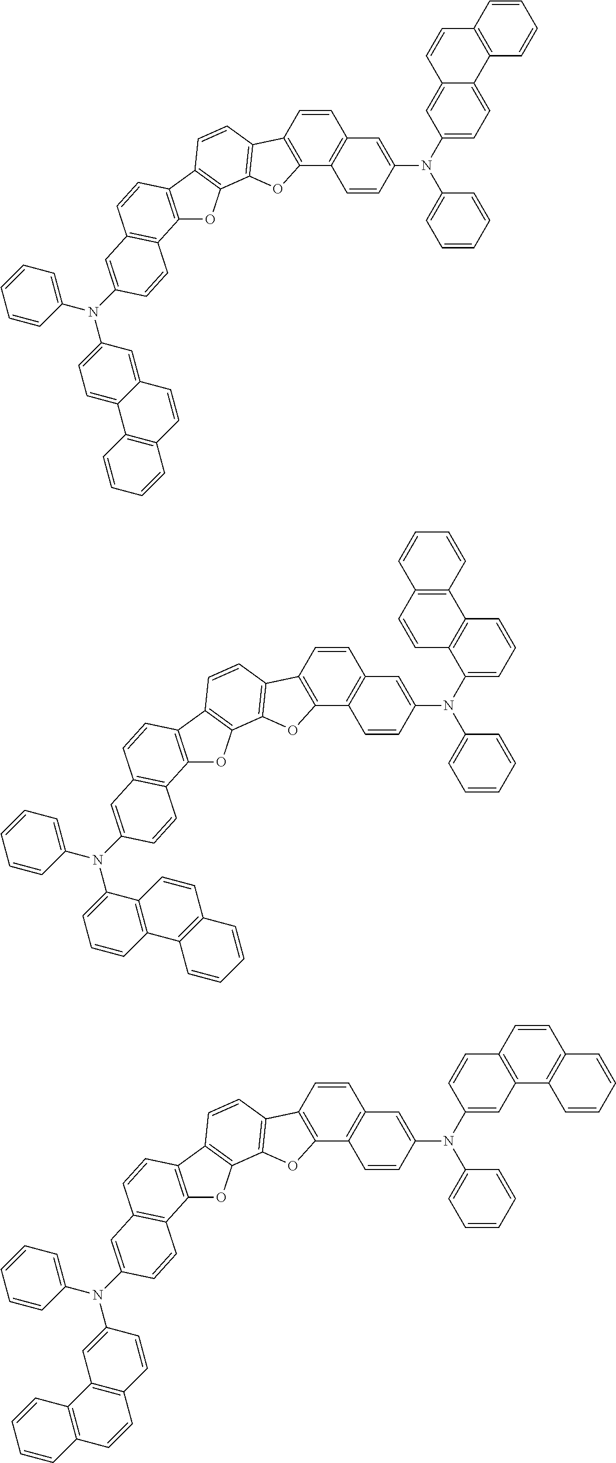 [SMILES: c1ccc(N(c2ccc3c(ccc4ccccc43)c2)c2ccc3c(ccc4c5ccc6c7ccc8cc(N(c9ccccc9)c9ccc%10c(ccc%11ccccc%11%10)c9)ccc8c7oc6c5oc34)c2)cc1 ; c1ccc(N(c2ccc3c(ccc4c5ccc6c7ccc8cc(N(c9ccccc9)c9cccc%10c9ccc9ccccc9%10)ccc8c7oc6c5oc34)c2)c2cccc3c2ccc2ccccc23)cc1 ; c1ccc(N(c2ccc3c(ccc4c5ccc6c7ccc8cc(N(c9ccccc9)c9ccc%10ccc%11ccccc%11c%10c9)ccc8c7oc6c5oc34)c2)c2ccc3ccc4ccccc4c3c2)cc1]

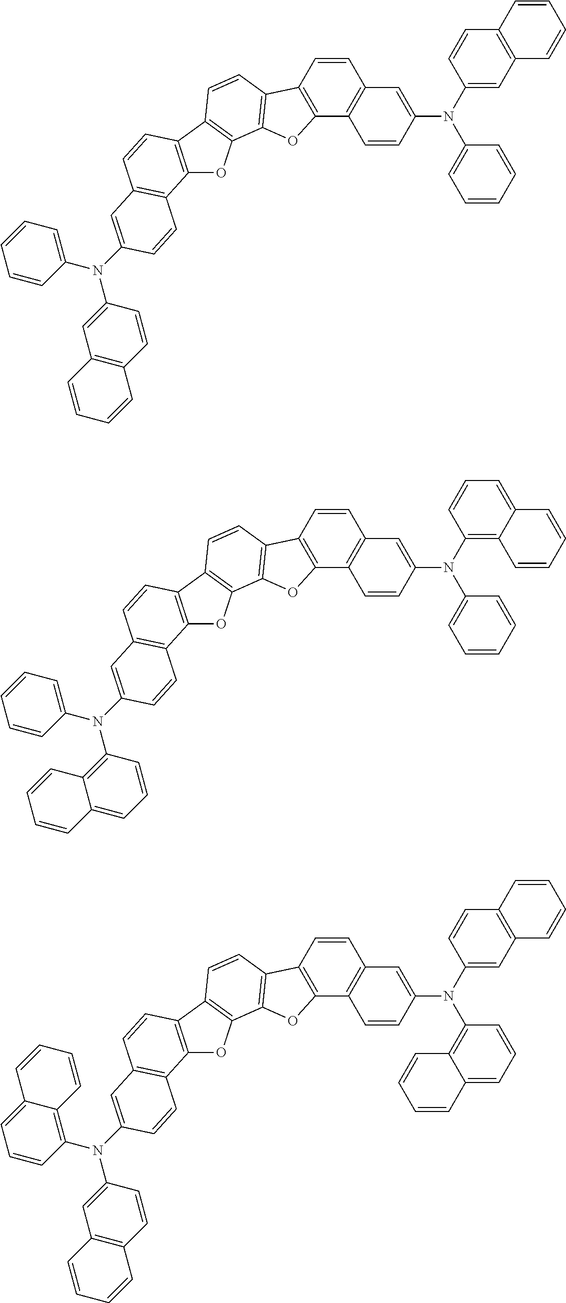 [SMILES: c1ccc(N(c2ccc3ccccc3c2)c2ccc3c(ccc4c5ccc6c7ccc8cc(N(c9ccccc9)c9ccc%10ccccc%10c9)ccc8c7oc6c5oc34)c2)cc1 ; c1ccc(N(c2ccc3c(ccc4c5ccc6c7ccc8cc(N(c9ccccc9)c9cccc%10ccccc9%10)ccc8c7oc6c5oc34)c2)c2cccc3ccccc23)cc1 ; c1ccc2cc(N(c3ccc4c(ccc5c6ccc7c8ccc9cc(N(c%10ccc%11ccccc%11c%10)c%10cccc%11ccccc%10%11)ccc9c8oc7c6oc45)c3)c3cccc4ccccc34)ccc2c1]

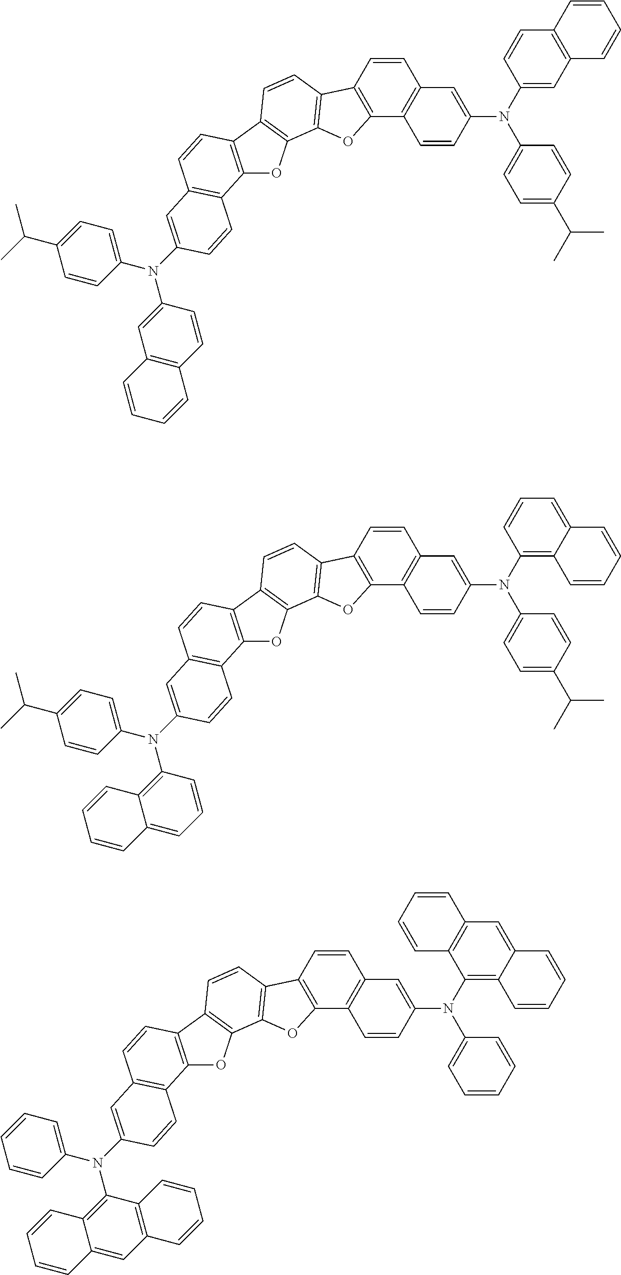 [SMILES: CC(C)c1ccc(N(c2ccc3ccccc3c2)c2ccc3c(ccc4c5ccc6c7ccc8cc(N(c9ccc(C(C)C)cc9)c9ccc%10ccccc%10c9)ccc8c7oc6c5oc34)c2)cc1 ; CC(C)c1ccc(N(c2ccc3c(ccc4c5ccc6c7ccc8cc(N(c9ccc(C(C)C)cc9)c9cccc%10ccccc9%10)ccc8c7oc6c5oc34)c2)c2cccc3ccccc23)cc1 ; c1ccc(N(c2ccc3c(ccc4c5ccc6c7ccc8cc(N(c9ccccc9)c9c%10ccccc%10cc%10ccccc9%10)ccc8c7oc6c5oc34)c2)c2c3ccccc3cc3ccccc23)cc1]

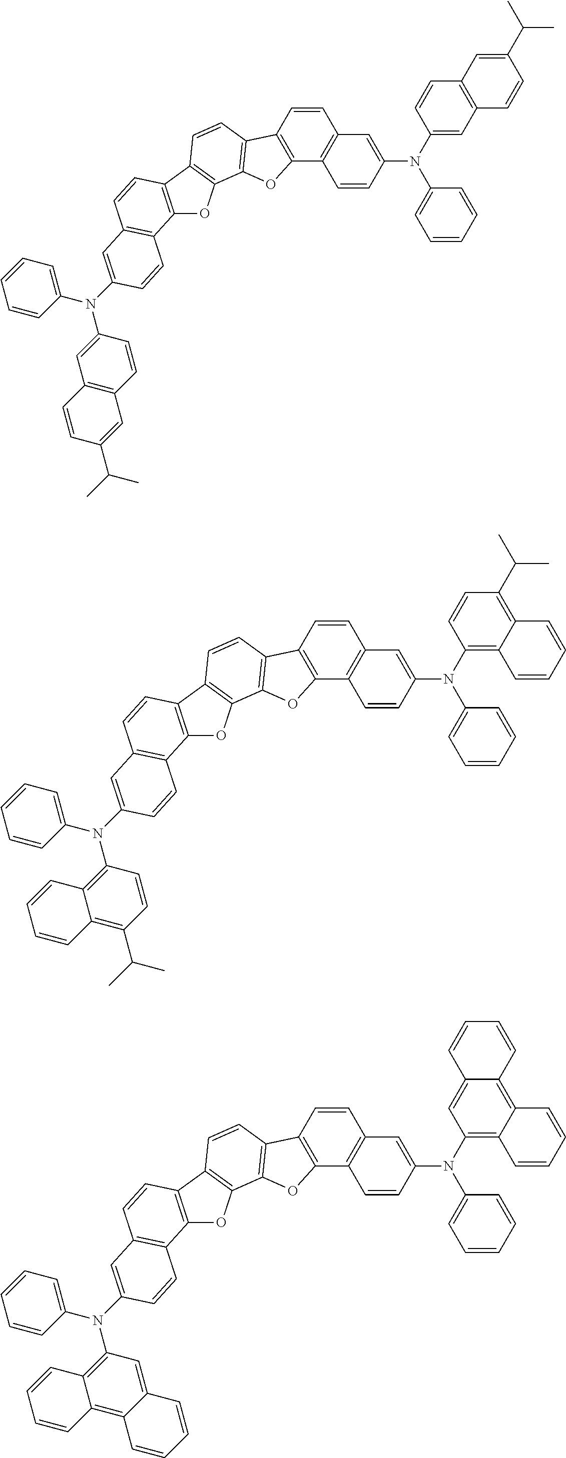 [SMILES: CC(C)c1ccc2cc(N(c3ccccc3)c3ccc4c(ccc5c6ccc7c8ccc9cc(N(c%10ccccc%10)c%10ccc%11cc(C(C)C)ccc%11c%10)ccc9c8oc7c6oc45)c3)ccc2c1 ; CC(C)c1ccc(N(c2ccccc2)c2ccc3c(ccc4c5ccc6c7ccc8cc(N(c9ccccc9)c9ccc(C(C)C)c%10ccccc9%10)ccc8c7oc6c5oc34)c2)c2ccccc12 ; c1ccc(N(c2ccc3c(ccc4c5ccc6c7ccc8cc(N(c9ccccc9)c9cc%10ccccc%10c%10ccccc9%10)ccc8c7oc6c5oc34)c2)c2cc3ccccc3c3ccccc23)cc1]

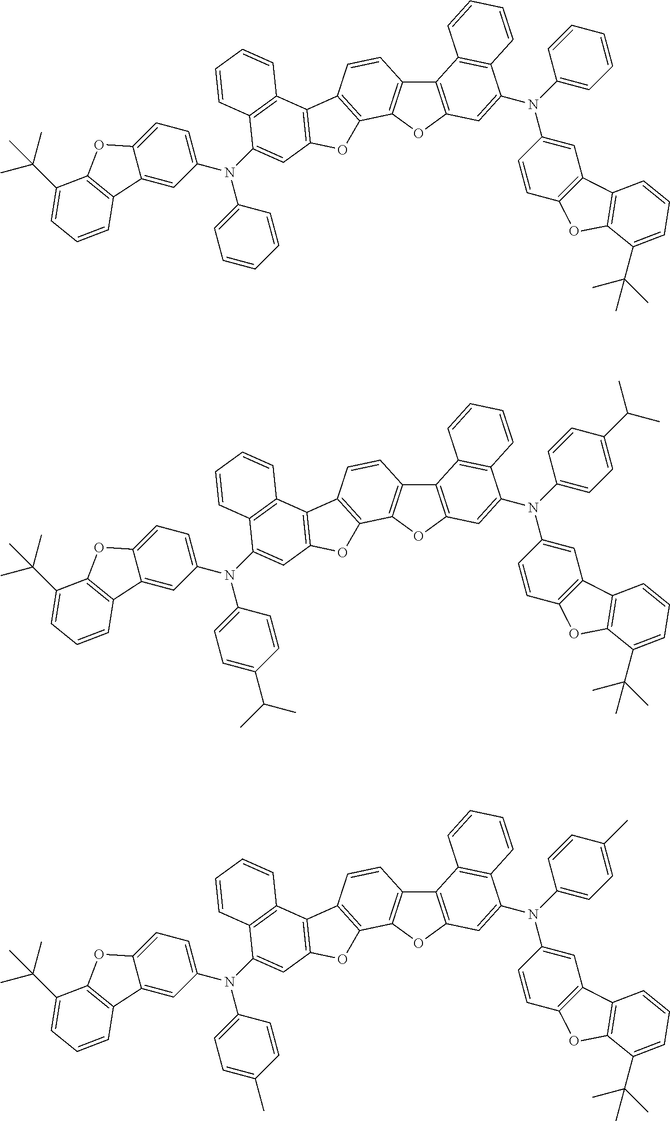 [SMILES: CC(C)(C)c1cccc2c1oc1ccc(N(c3ccccc3)c3cc4oc5c(ccc6c5oc5cc(N(c7ccccc7)c7ccc8oc9c(C(C)(C)C)cccc9c8c7)c7ccccc7c56)c4c4ccccc34)cc12 ; CC(C)c1ccc(N(c2ccc3oc4c(C(C)(C)C)cccc4c3c2)c2cc3oc4c(ccc5c4oc4cc(N(c6ccc(C(C)C)cc6)c6ccc7oc8c(C(C)(C)C)cccc8c7c6)c6ccccc6c45)c3c3ccccc23)cc1 ; Cc1ccc(N(c2ccc3oc4c(C(C)(C)C)cccc4c3c2)c2cc3oc4c(ccc5c4oc4cc(N(c6ccc(C)cc6)c6ccc7oc8c(C(C)(C)C)cccc8c7c6)c6ccccc6c45)c3c3ccccc23)cc1]

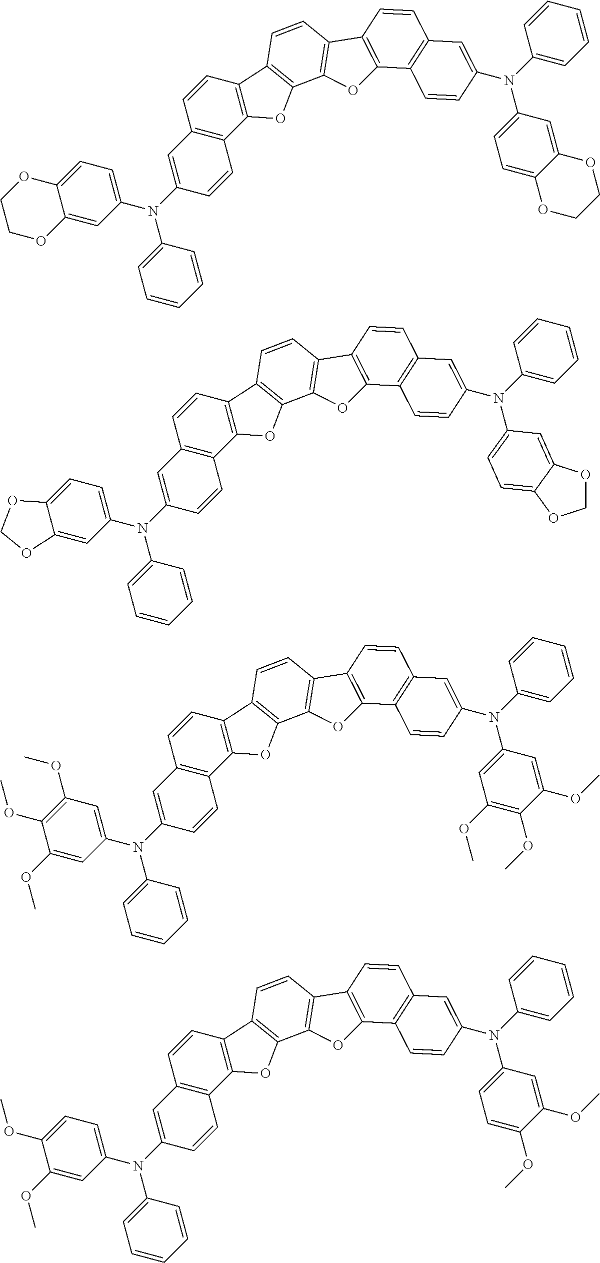

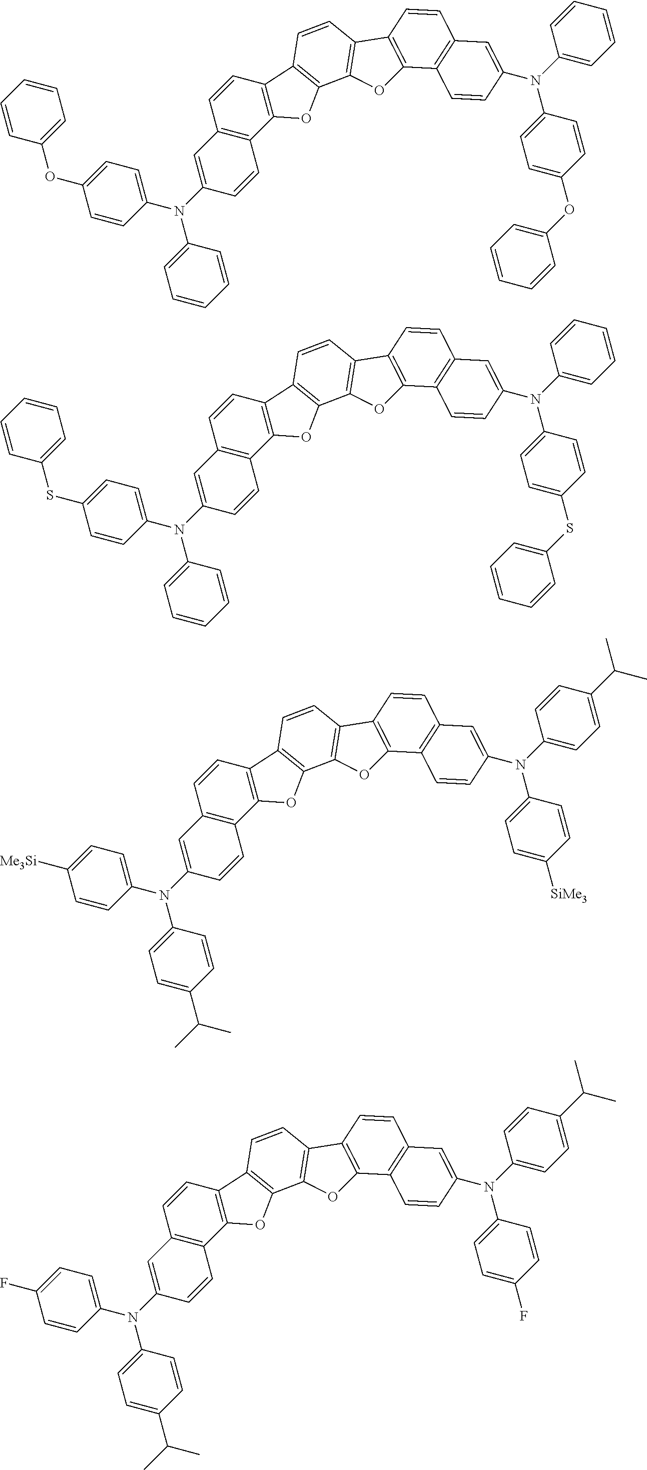 [SMILES: c1ccc(Oc2ccc(N(c3ccccc3)c3ccc4c(ccc5c6ccc7c8ccc9cc(N(c%10ccccc%10)c%10ccc(Oc%11ccccc%11)cc%10)ccc9c8oc7c6oc45)c3)cc2)cc1 ; c1ccc(Sc2ccc(N(c3ccccc3)c3ccc4c(ccc5c6ccc7c8ccc9cc(N(c%10ccccc%10)c%10ccc(Sc%11ccccc%11)cc%10)ccc9c8oc7c6oc45)c3)cc2)cc1 ; CC(C)c1ccc(N(c2ccc([Si](C)(C)C)cc2)c2ccc3c(ccc4c5ccc6c7ccc8cc(N(c9ccc(C(C)C)cc9)c9ccc([Si](C)(C)C)cc9)ccc8c7oc6c5oc34)c2)cc1 ; CC(C)c1ccc(N(c2ccc(F)cc2)c2ccc3c(ccc4c5ccc6c7ccc8cc(N(c9ccc(F)cc9)c9ccc(C(C)C)cc9)ccc8c7oc6c5oc34)c2)cc1]

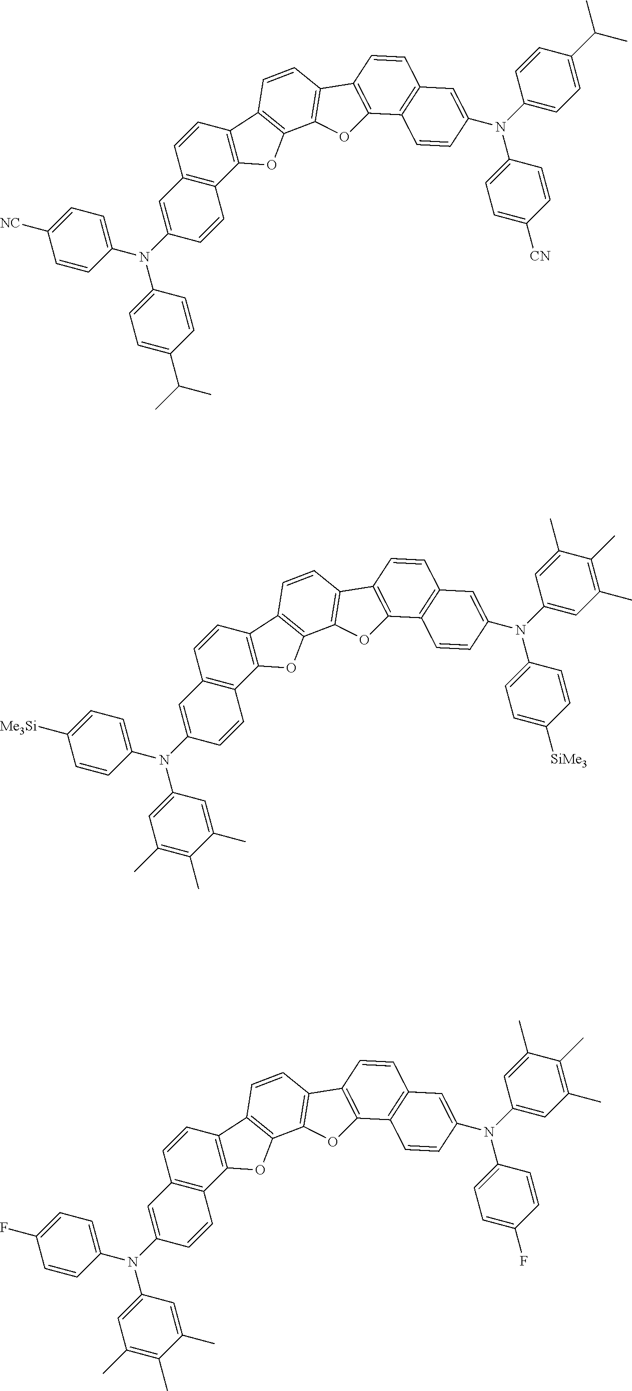 [SMILES: CC(C)c1ccc(N(c2ccc(C#N)cc2)c2ccc3c(ccc4c5ccc6c7ccc8cc(N(c9ccc(C#N)cc9)c9ccc(C(C)C)cc9)ccc8c7oc6c5oc34)c2)cc1 ; Cc1cc(N(c2ccc([Si](C)(C)C)cc2)c2ccc3c(ccc4c5ccc6c7ccc8cc(N(c9ccc([Si](C)(C)C)cc9)c9cc(C)c(C)c(C)c9)ccc8c7oc6c5oc34)c2)cc(C)c1C ; Cc1cc(N(c2ccc(F)cc2)c2ccc3c(ccc4c5ccc6c7ccc8cc(N(c9ccc(F)cc9)c9cc(C)c(C)c(C)c9)ccc8c7oc6c5oc34)c2)cc(C)c1C]

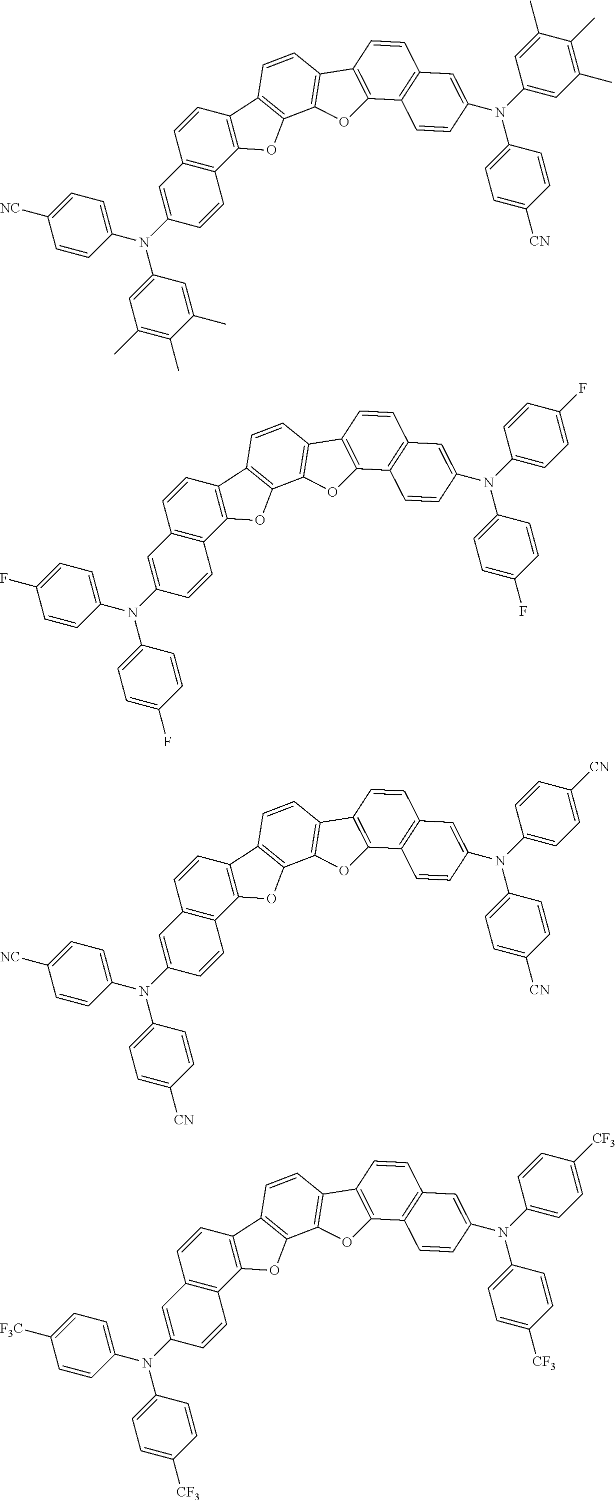 [SMILES: Cc1cc(N(c2ccc(C#N)cc2)c2ccc3c(ccc4c5ccc6c7ccc8cc(N(c9ccc(C#N)cc9)c9cc(C)c(C)c(C)c9)ccc8c7oc6c5oc34)c2)cc(C)c1C ; Fc1ccc(N(c2ccc(F)cc2)c2ccc3c(ccc4c5ccc6c7ccc8cc(N(c9ccc(F)cc9)c9ccc(F)cc9)ccc8c7oc6c5oc34)c2)cc1 ; N#Cc1ccc(N(c2ccc(C#N)cc2)c2ccc3c(ccc4c5ccc6c7ccc8cc(N(c9ccc(C#N)cc9)c9ccc(C#N)cc9)ccc8c7oc6c5oc34)c2)cc1 ; FC(F)(F)c1ccc(N(c2ccc(C(F)(F)F)cc2)c2ccc3c(ccc4c5ccc6c7ccc8cc(N(c9ccc(C(F)(F)F)cc9)c9ccc(C(F)(F)F)cc9)ccc8c7oc6c5oc34)c2)cc1]

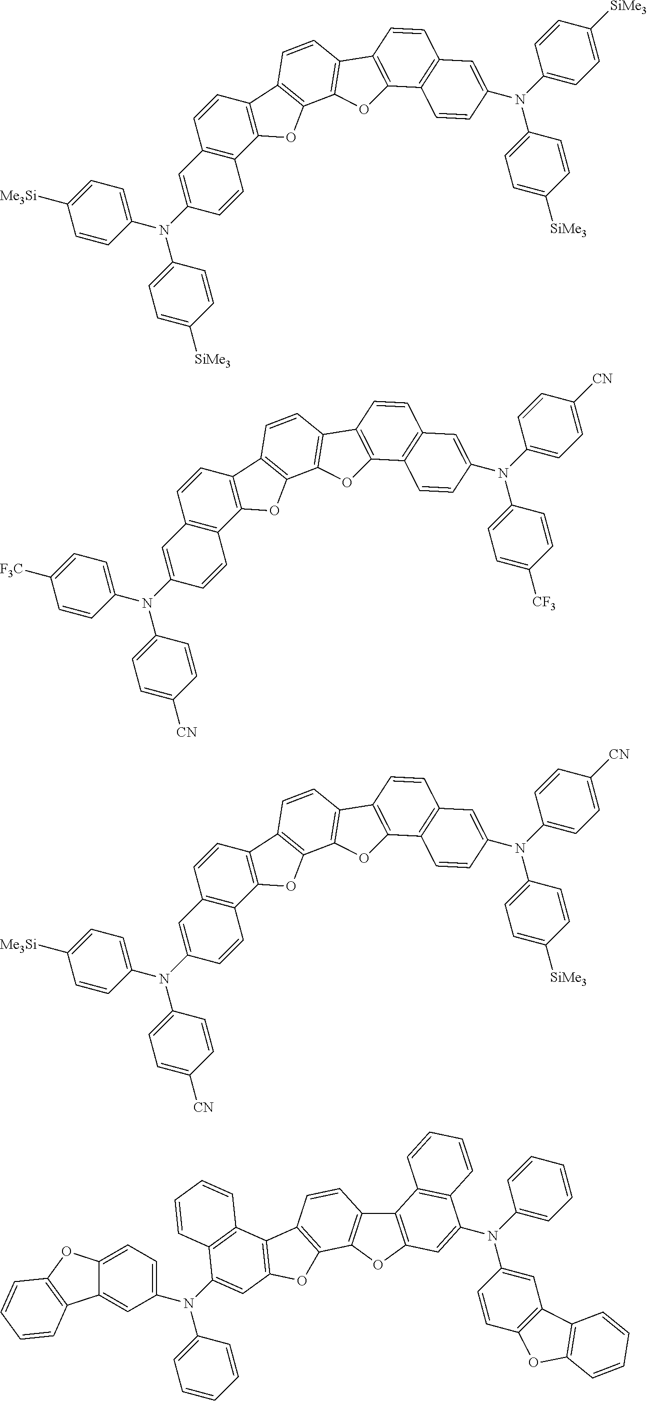 [SMILES: C[Si](C)(C)c1ccc(N(c2ccc([Si](C)(C)C)cc2)c2ccc3c(ccc4c5ccc6c7ccc8cc(N(c9ccc([Si](C)(C)C)cc9)c9ccc([Si](C)(C)C)cc9)ccc8c7oc6c5oc34)c2)cc1 ; N#Cc1ccc(N(c2ccc(C(F)(F)F)cc2)c2ccc3c(ccc4c5ccc6c7ccc8cc(N(c9ccc(C#N)cc9)c9ccc(C(F)(F)F)cc9)ccc8c7oc6c5oc34)c2)cc1 ; C[Si](C)(C)c1ccc(N(c2ccc(C#N)cc2)c2ccc3c(ccc4c5ccc6c7ccc8cc(N(c9ccc(C#N)cc9)c9ccc([Si](C)(C)C)cc9)ccc8c7oc6c5oc34)c2)cc1 ; c1ccc(N(c2ccc3oc4ccccc4c3c2)c2cc3oc4c(ccc5c4oc4cc(N(c6ccccc6)c6ccc7oc8ccccc8c7c6)c6ccccc6c45)c3c3ccccc23)cc1]

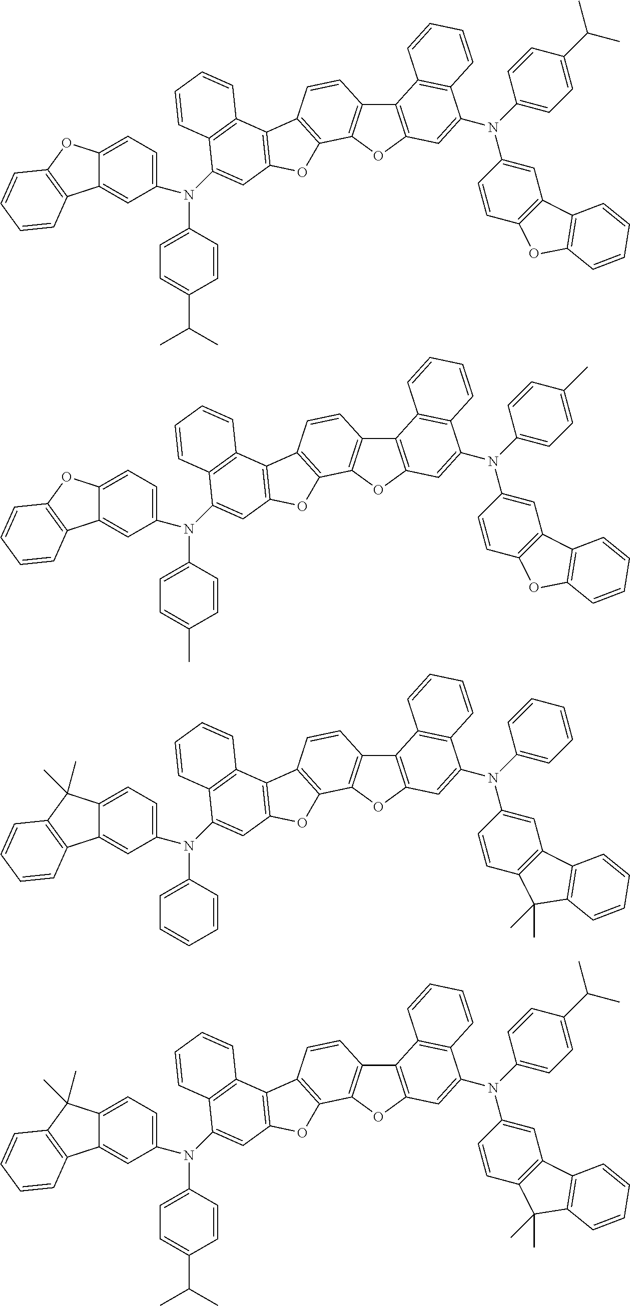

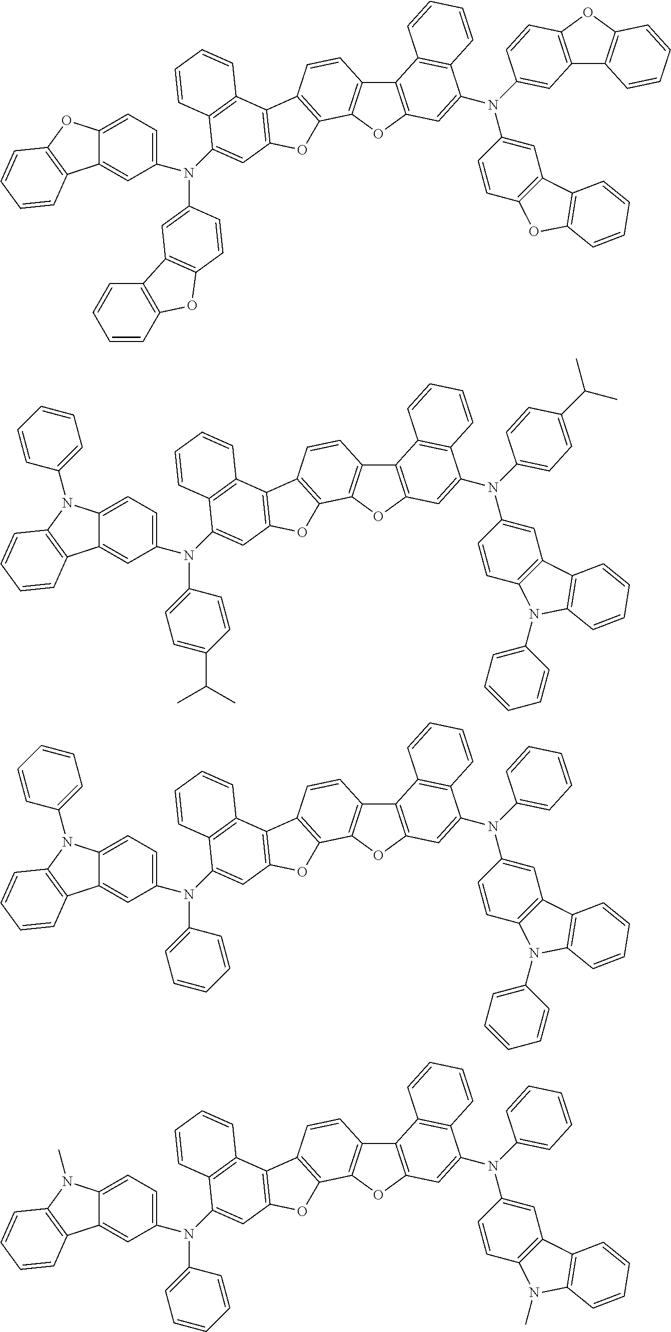

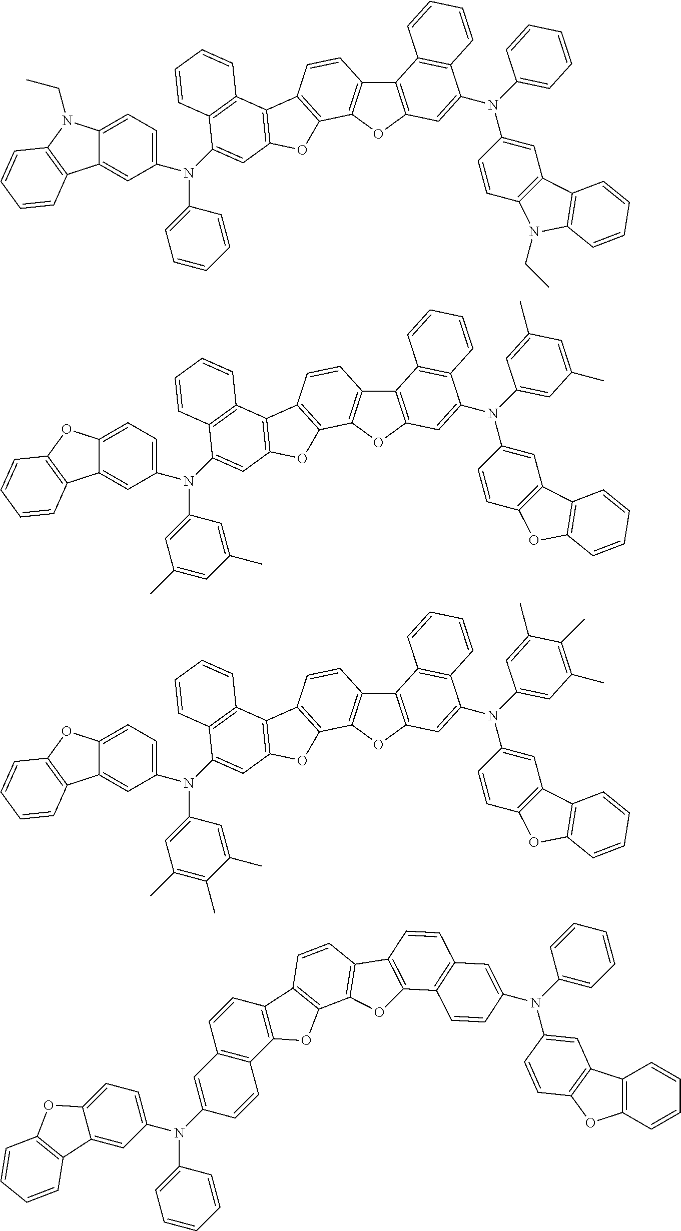

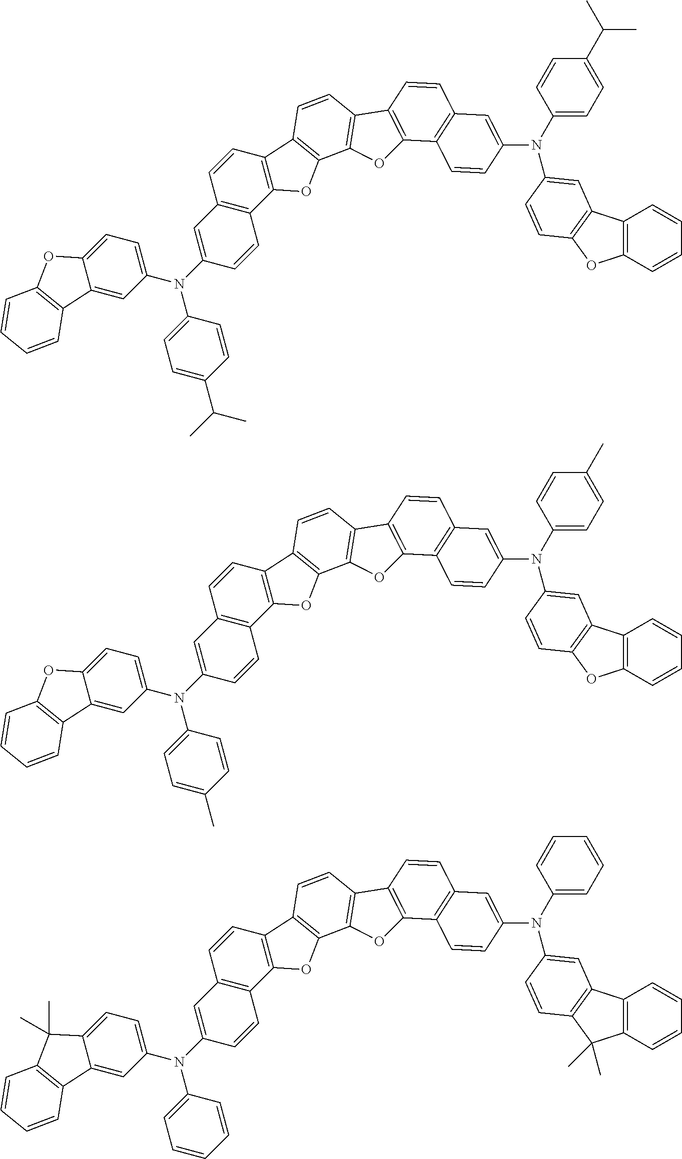 [SMILES: CC(C)c1ccc(N(c2ccc3c(ccc4c5ccc6c7ccc8cc(N(c9ccc(C(C)C)cc9)c9ccc%10oc%11ccccc%11c%10c9)ccc8c7oc6c5oc34)c2)c2ccc3oc4ccccc4c3c2)cc1 ; Cc1ccc(N(c2ccc3c(ccc4c5ccc6c7ccc8cc(N(c9ccc(C)cc9)c9ccc%10oc%11ccccc%11c%10c9)ccc8c7oc6c5oc34)c2)c2ccc3oc4ccccc4c3c2)cc1 ; CC1(C)c2ccccc2-c2cc(N(c3ccccc3)c3ccc4c(ccc5c6ccc7c8ccc9cc(N(c%10ccccc%10)c%10ccc%11c(c%10)-c%10ccccc%10C%11(C)C)ccc9c8oc7c6oc45)c3)ccc21]

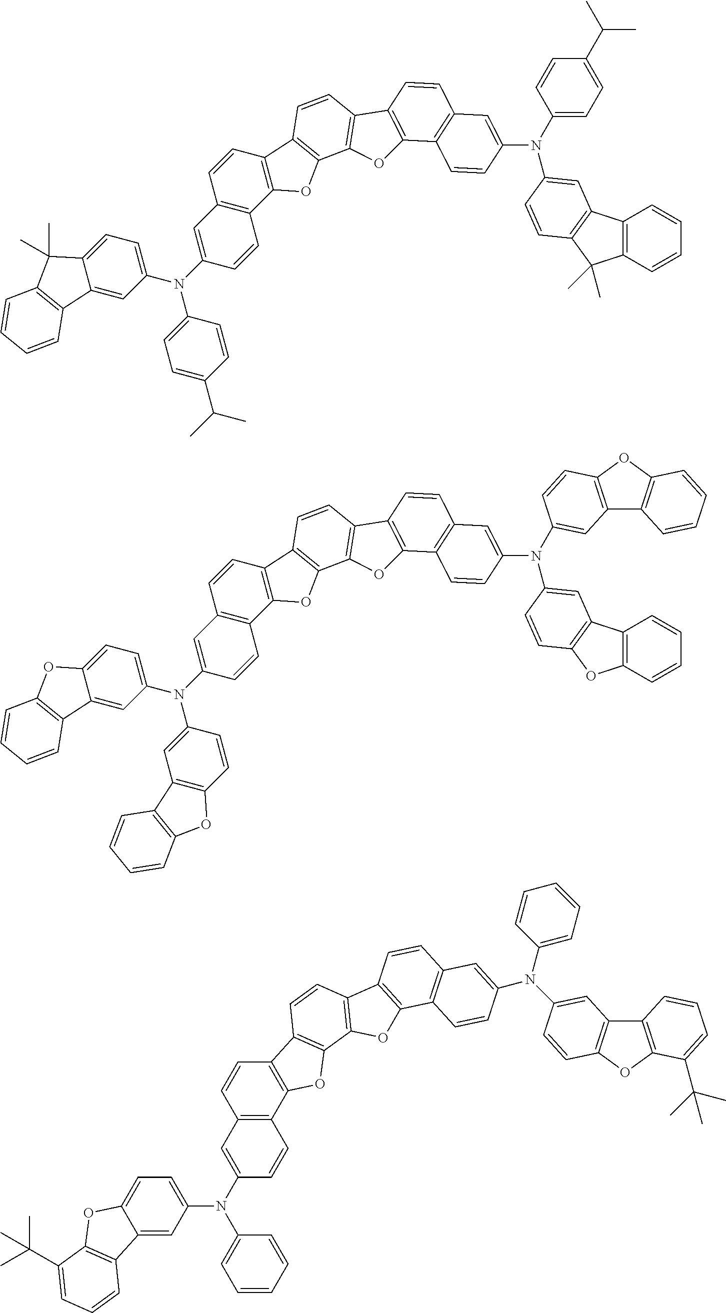 [SMILES: CC(C)c1ccc(N(c2ccc3c(c2)-c2ccccc2C3(C)C)c2ccc3c(ccc4c5ccc6c7ccc8cc(N(c9ccc(C(C)C)cc9)c9ccc%10c(c9)-c9ccccc9C%10(C)C)ccc8c7oc6c5oc34)c2)cc1 ; c1ccc2c(c1)oc1ccc(N(c3ccc4c(ccc5c6ccc7c8ccc9cc(N(c%10ccc%11oc%12ccccc%12c%11c%10)c%10ccc%11oc%12ccccc%12c%11c%10)ccc9c8oc7c6oc45)c3)c3ccc4oc5ccccc5c4c3)cc12 ; CC(C)(C)c1cccc2c1oc1ccc(N(c3ccccc3)c3ccc4c(ccc5c6ccc7c8ccc9cc(N(c%10ccccc%10)c%10ccc%11oc%12c(C(C)(C)C)cccc%12c%11c%10)ccc9c8oc7c6oc45)c3)cc12]

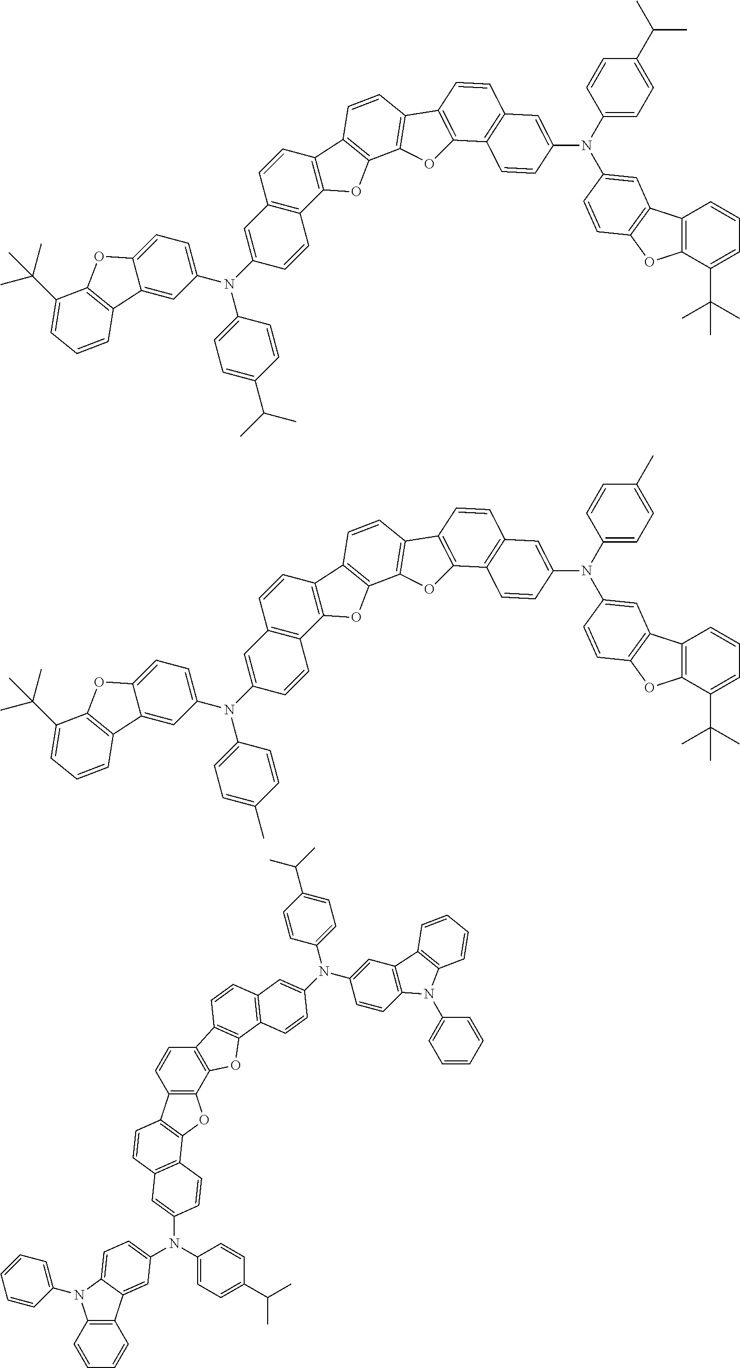

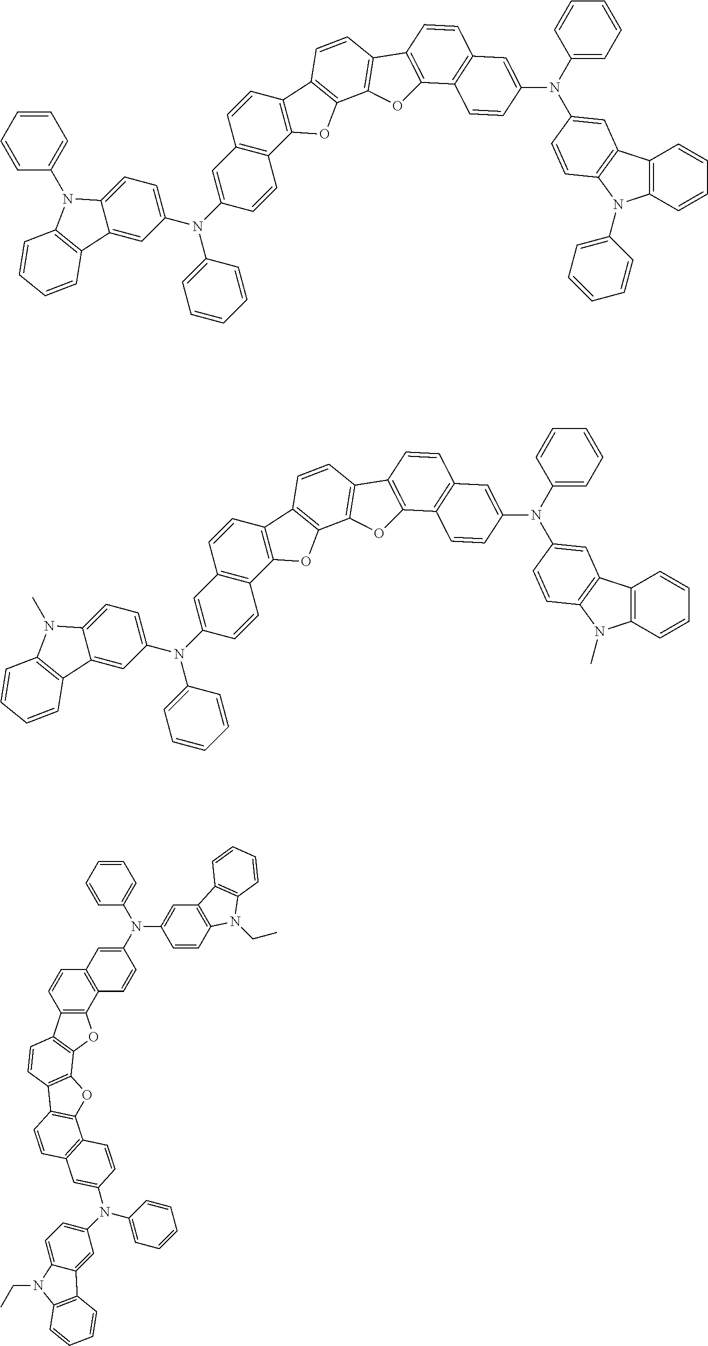 [SMILES: c1ccc(N(c2ccc3c(ccc4c5ccc6c7ccc8cc(N(c9ccccc9)c9ccc%10c(c9)c9ccccc9n%10-c9ccccc9)ccc8c7oc6c5oc34)c2)c2ccc3c(c2)c2ccccc2n3-c2ccccc2)cc1 ; Cn1c2ccccc2c2cc(N(c3ccccc3)c3ccc4c(ccc5c6ccc7c8ccc9cc(N(c%10ccccc%10)c%10ccc%11c(c%10)c%10ccccc%10n%11C)ccc9c8oc7c6oc45)c3)ccc21 ; CCn1c2ccccc2c2cc(N(c3ccccc3)c3ccc4c(ccc5c6ccc7c8ccc9cc(N(c%10ccccc%10)c%10ccc%11c(c%10)c%10ccccc%10n%11CC)ccc9c8oc7c6oc45)c3)ccc21]

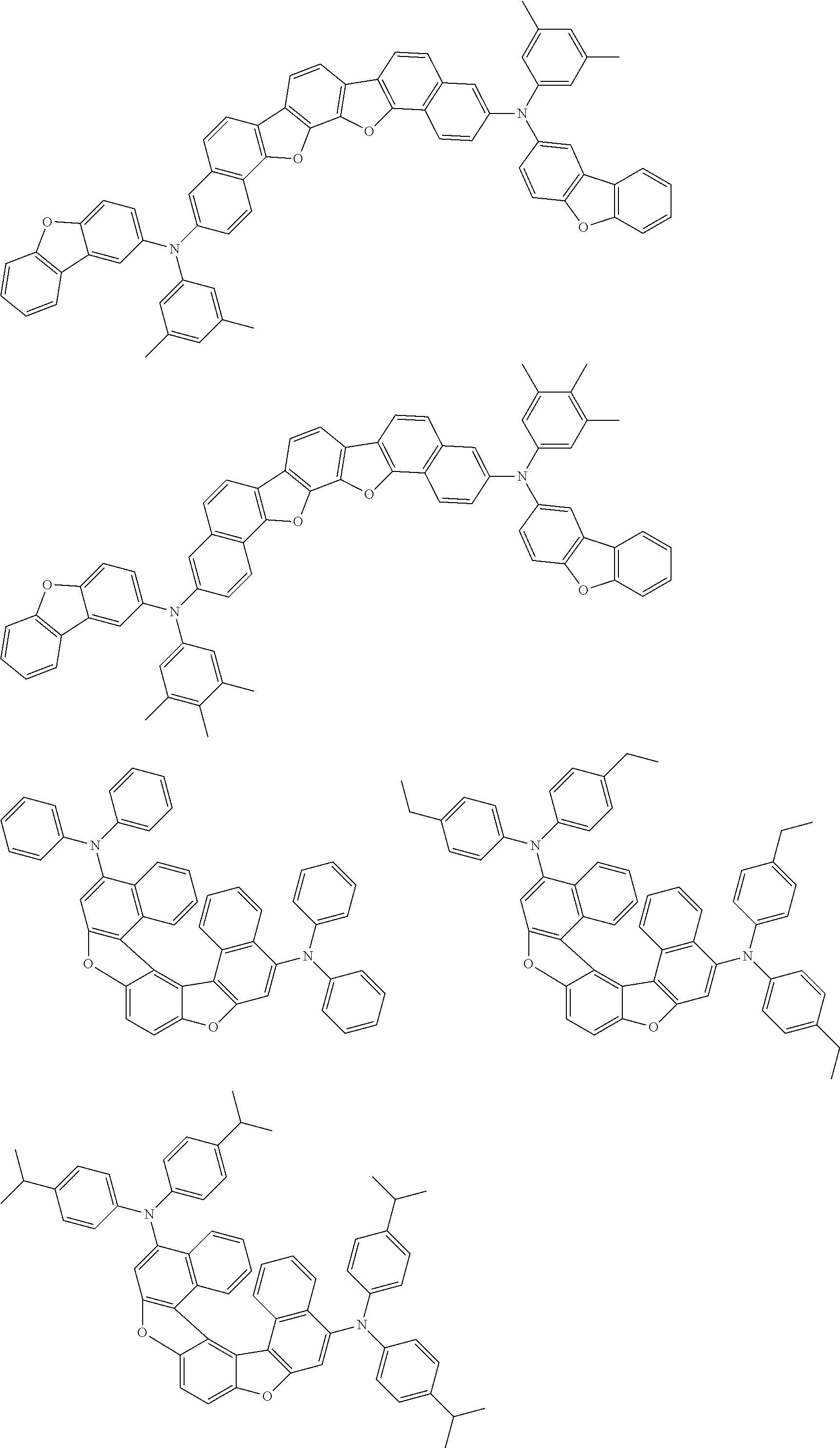

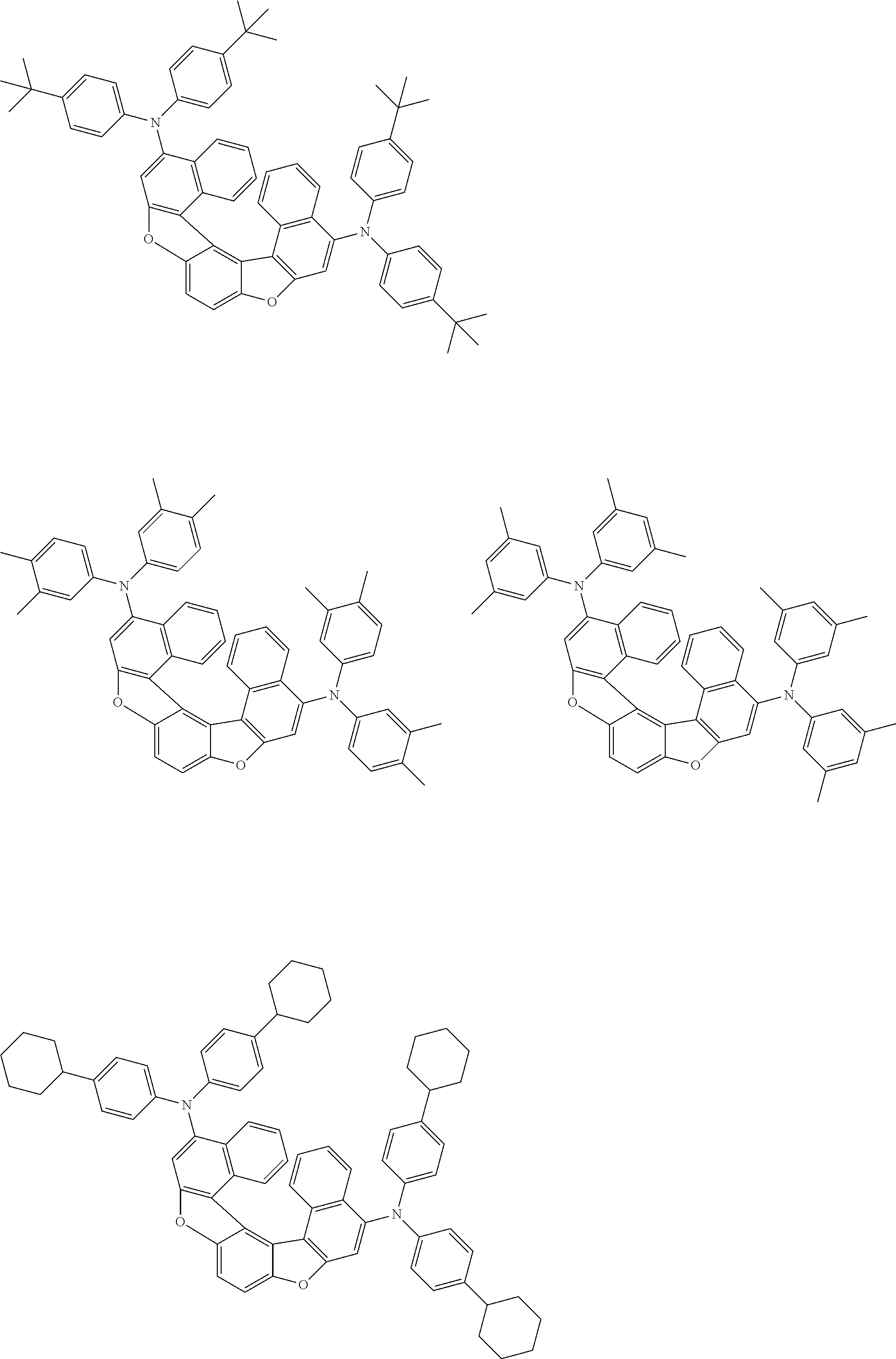

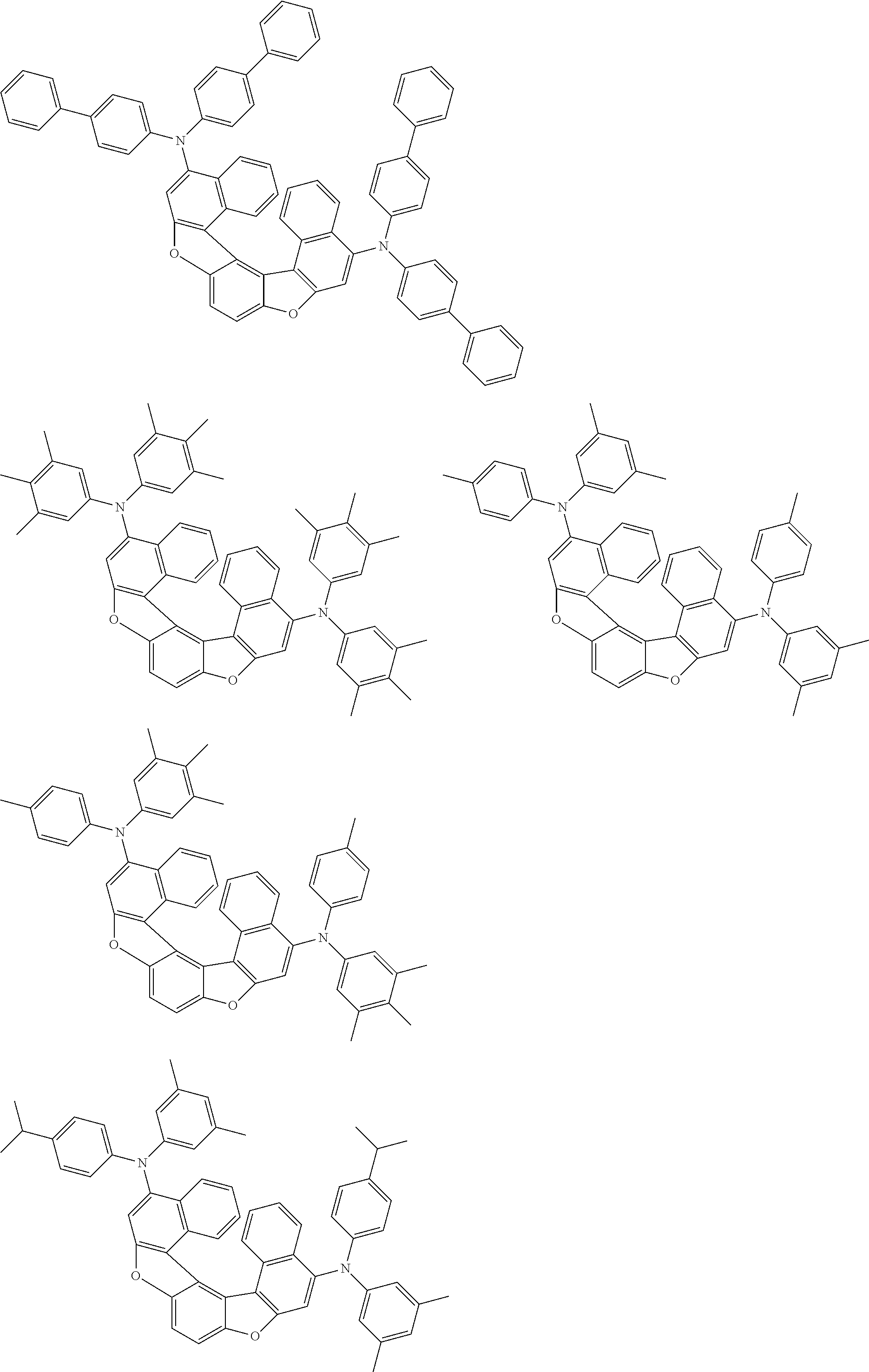

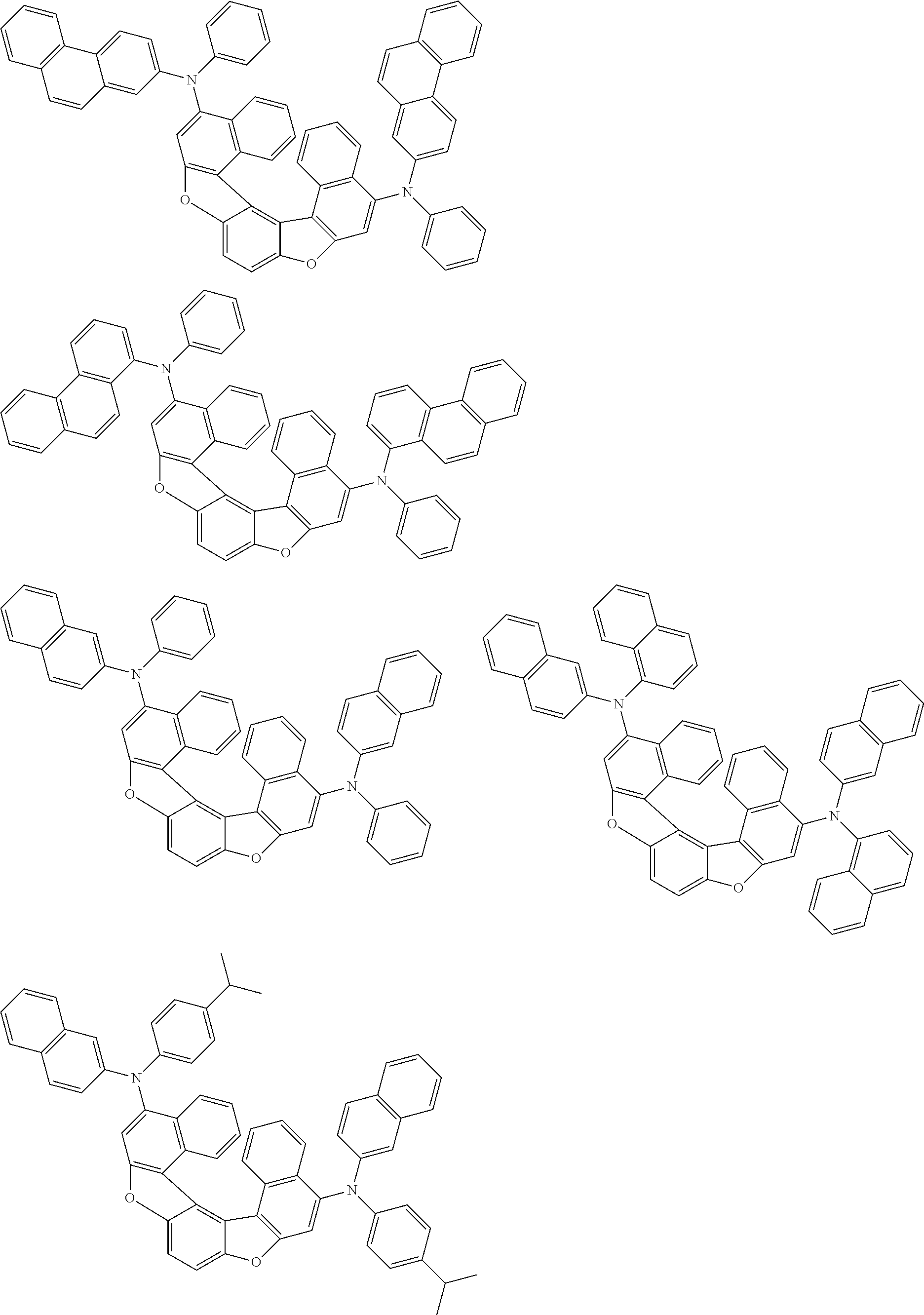

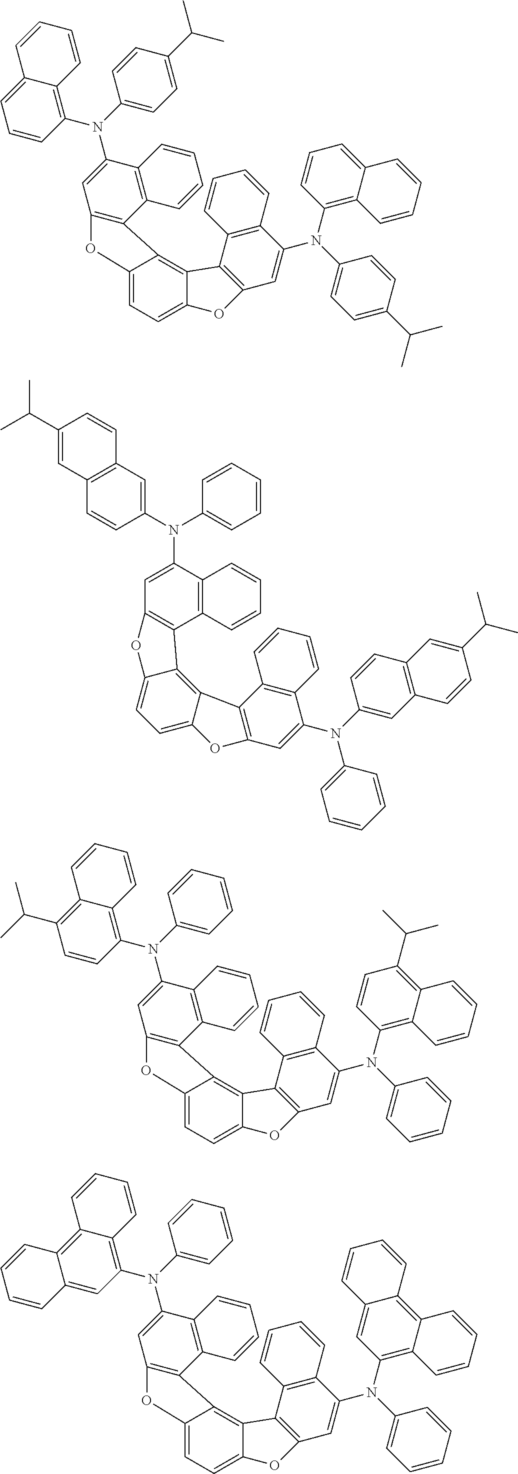

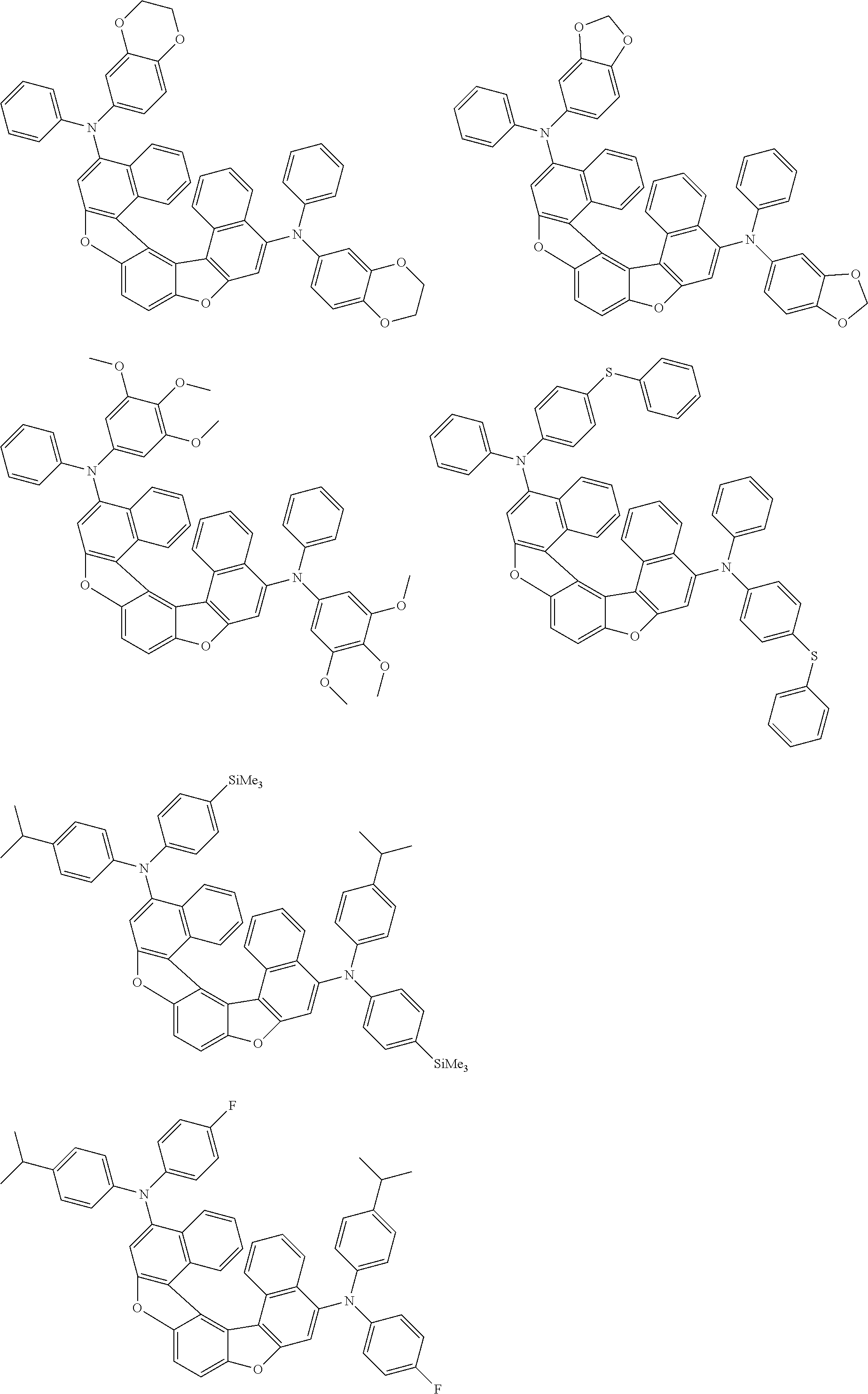

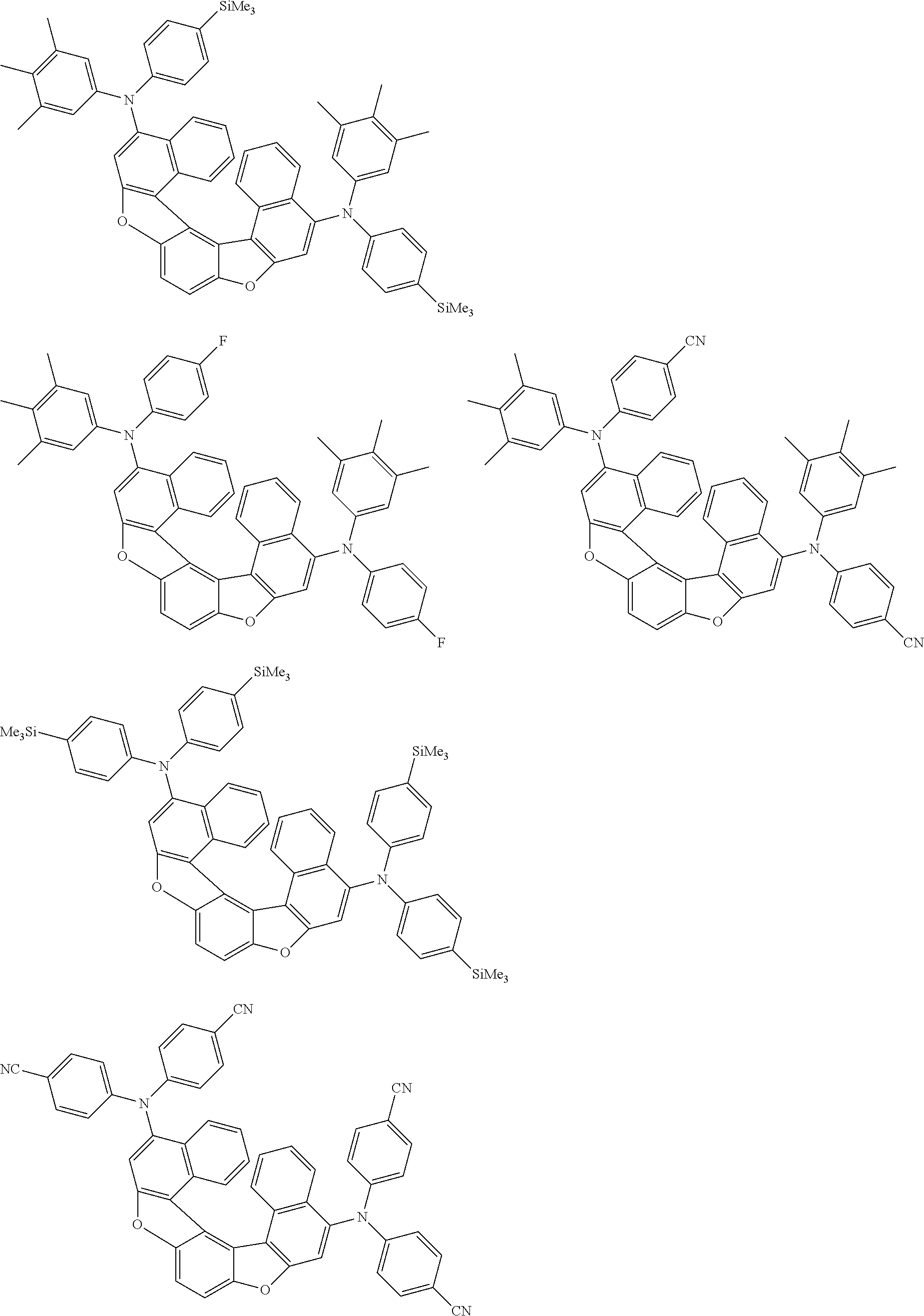

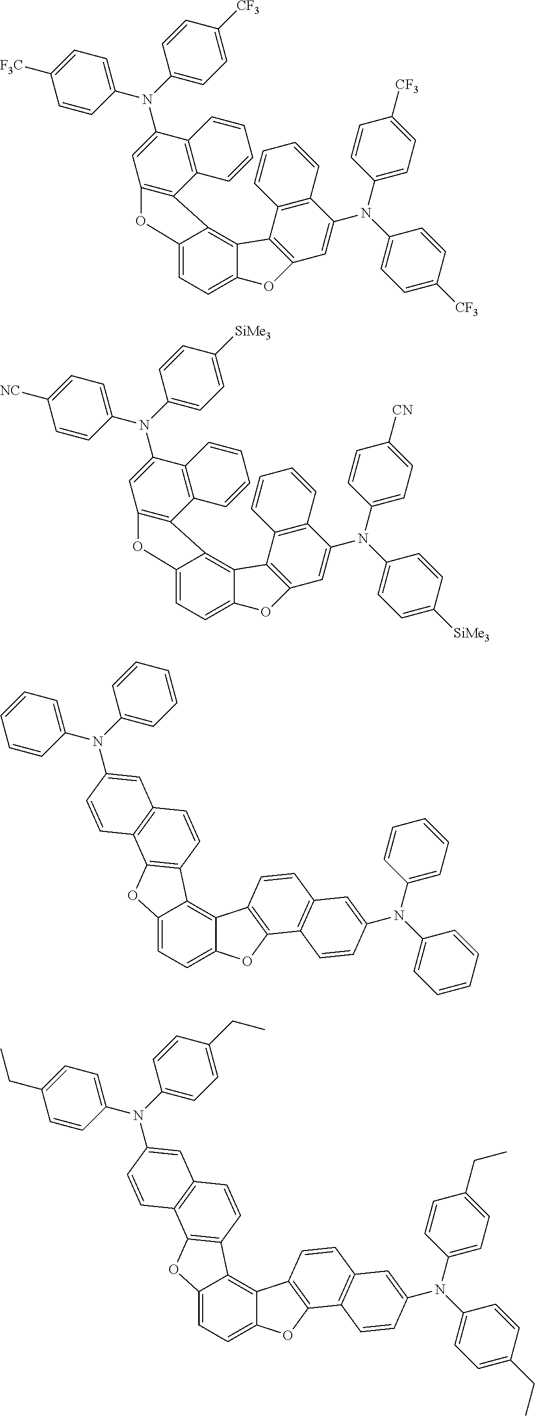 [SMILES: FC(F)(F)c1ccc(N(c2ccc(C(F)(F)F)cc2)c2cc3oc4ccc5oc6cc(N(c7ccc(C(F)(F)F)cc7)c7ccc(C(F)(F)F)cc7)c7ccccc7c6c5c4c3c3ccccc23)cc1 ; C[Si](C)(C)c1ccc(N(c2ccc(C#N)cc2)c2cc3oc4ccc5oc6cc(N(c7ccc(C#N)cc7)c7ccc([Si](C)(C)C)cc7)c7ccccc7c6c5c4c3c3ccccc23)cc1 ; c1ccc(N(c2ccccc2)c2ccc3c(ccc4c3oc3ccc5oc6c7ccc(N(c8ccccc8)c8ccccc8)cc7ccc6c5c34)c2)cc1 ; CCc1ccc(N(c2ccc(CC)cc2)c2ccc3c(ccc4c3oc3ccc5oc6c7ccc(N(c8ccc(CC)cc8)c8ccc(CC)cc8)cc7ccc6c5c34)c2)cc1]

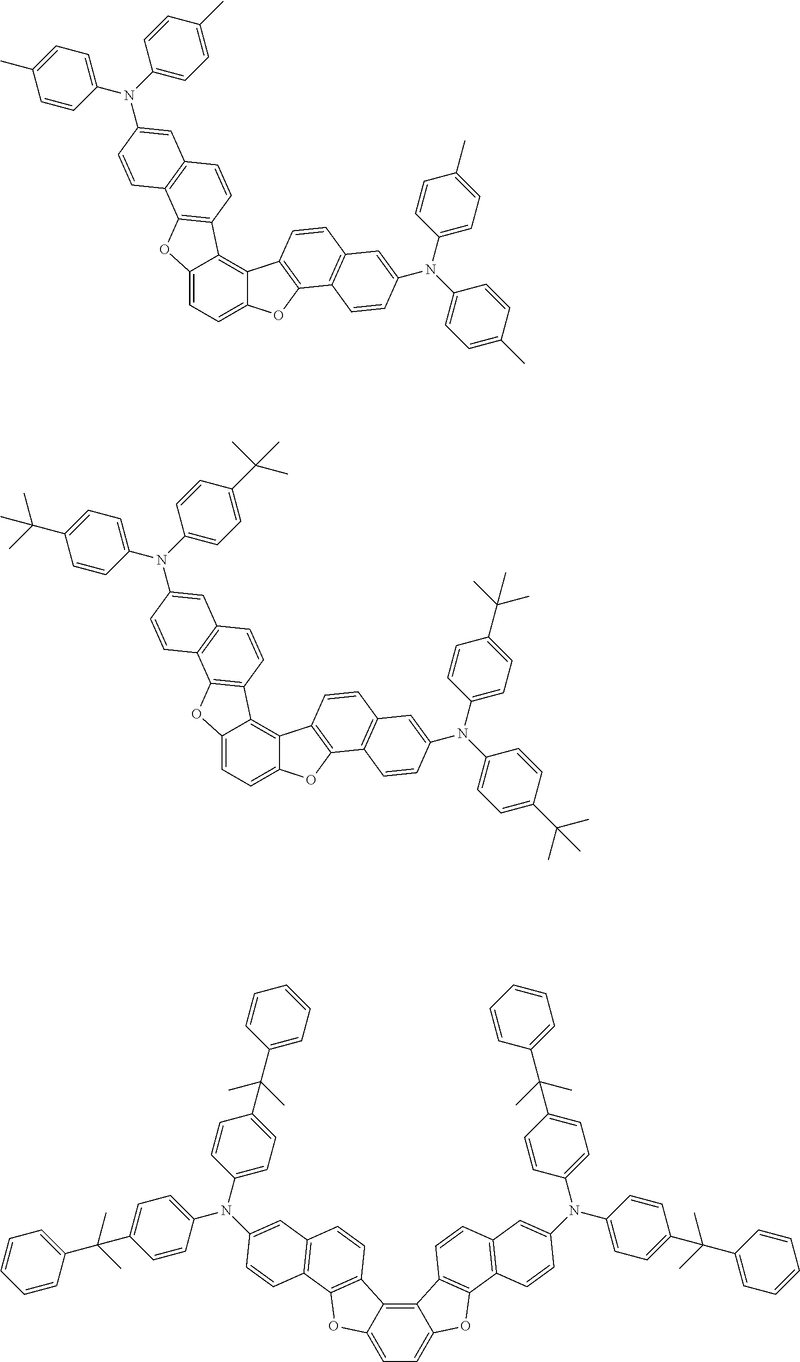

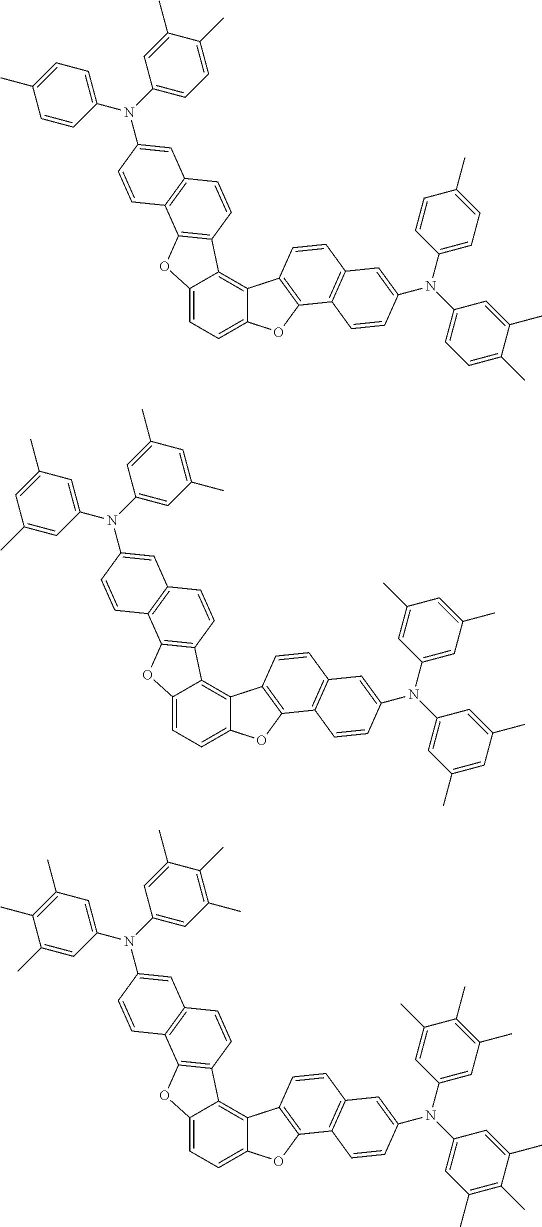

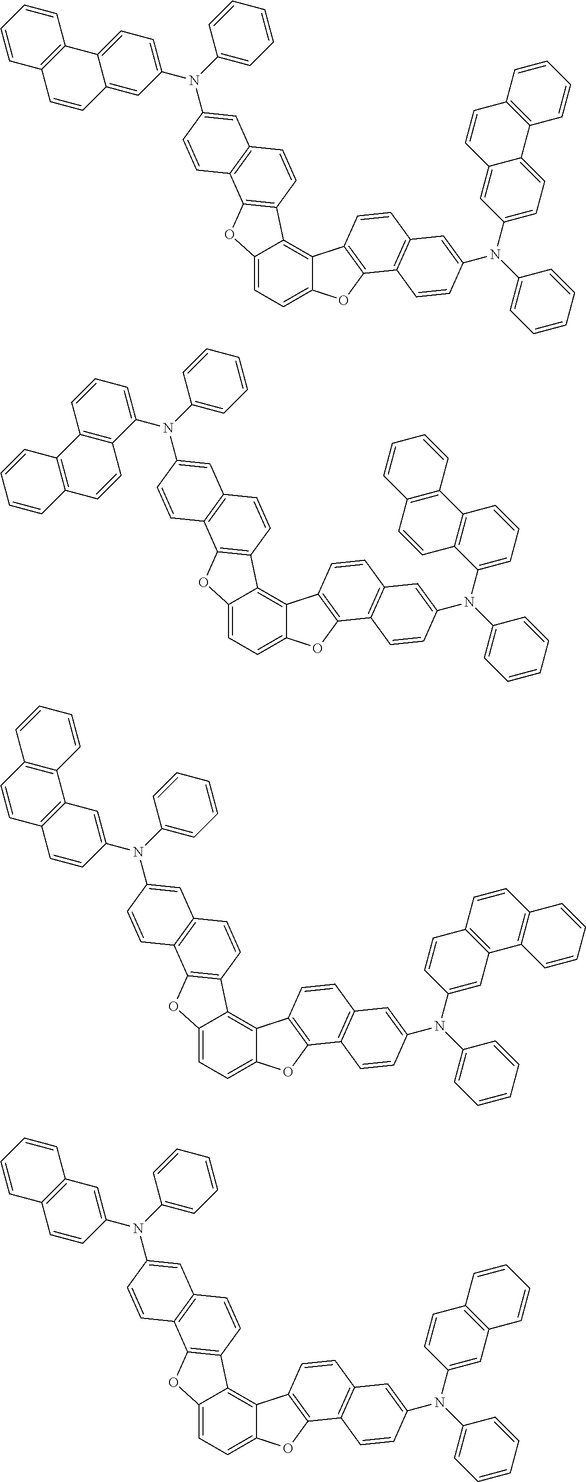 [SMILES: c1ccc(N(c2ccc3c(ccc4ccccc43)c2)c2ccc3c(ccc4c3oc3ccc5oc6c7ccc(N(c8ccccc8)c8ccc9c(ccc%10ccccc%109)c8)cc7ccc6c5c34)c2)cc1 ; c1ccc(N(c2ccc3c(ccc4c3oc3ccc5oc6c7ccc(N(c8ccccc8)c8cccc9c8ccc8ccccc89)cc7ccc6c5c34)c2)c2cccc3c2ccc2ccccc23)cc1 ; c1ccc(N(c2ccc3c(ccc4c3oc3ccc5oc6c7ccc(N(c8ccccc8)c8ccc9ccc%10ccccc%10c9c8)cc7ccc6c5c34)c2)c2ccc3ccc4ccccc4c3c2)cc1 ; c1ccc(N(c2ccc3ccccc3c2)c2ccc3c(ccc4c3oc3ccc5oc6c7ccc(N(c8ccccc8)c8ccc9ccccc9c8)cc7ccc6c5c34)c2)cc1]

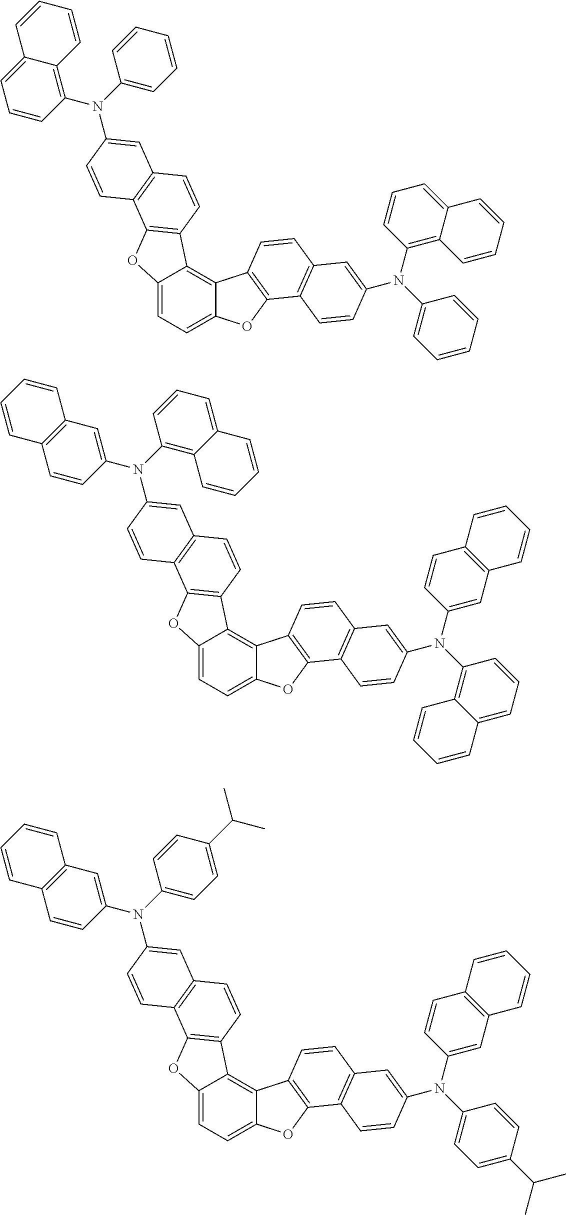 [SMILES: c1ccc(N(c2ccc3c(ccc4c3oc3ccc5oc6c7ccc(N(c8ccccc8)c8cccc9ccccc89)cc7ccc6c5c34)c2)c2cccc3ccccc23)cc1 ; c1ccc2cc(N(c3ccc4c(ccc5c4oc4ccc6oc7c8ccc(N(c9ccc%10ccccc%10c9)c9cccc%10ccccc9%10)cc8ccc7c6c45)c3)c3cccc4ccccc34)ccc2c1 ; CC(C)c1ccc(N(c2ccc3ccccc3c2)c2ccc3c(ccc4c3oc3ccc5oc6c7ccc(N(c8ccc(C(C)C)cc8)c8ccc9ccccc9c8)cc7ccc6c5c34)c2)cc1]

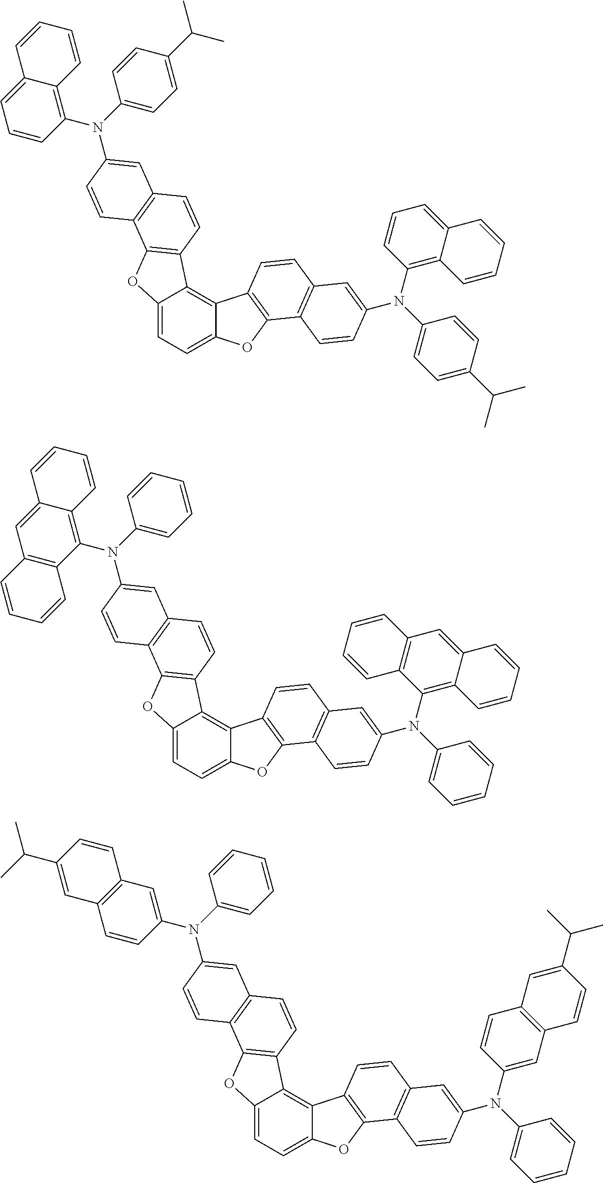

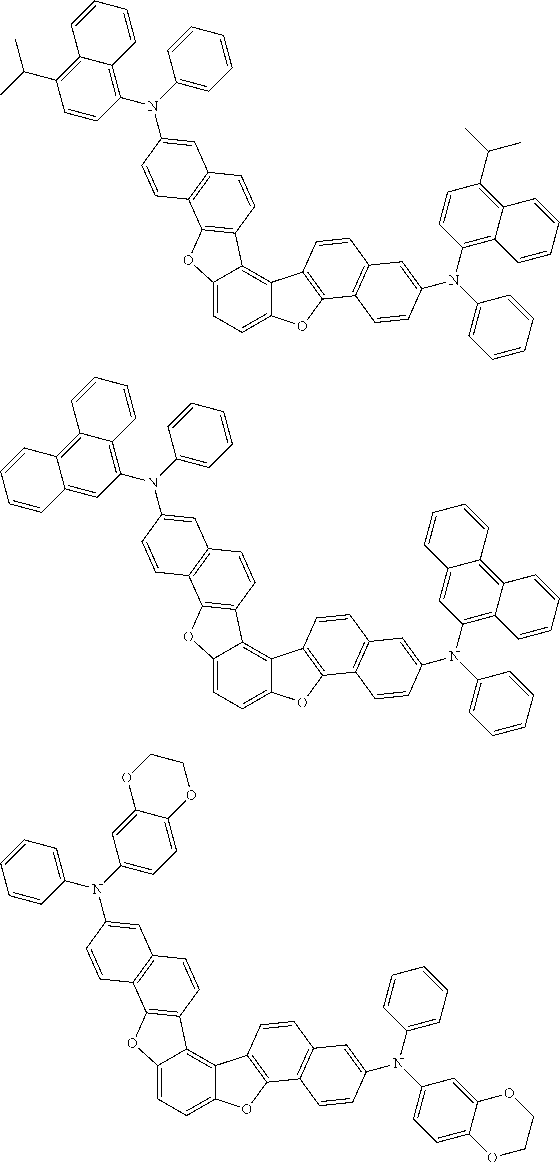 [SMILES: CC(C)c1ccc(N(c2ccccc2)c2ccc3c(ccc4c3oc3ccc5oc6c7ccc(N(c8ccccc8)c8ccc(C(C)C)c9ccccc89)cc7ccc6c5c34)c2)c2ccccc12 ; c1ccc(N(c2ccc3c(ccc4c3oc3ccc5oc6c7ccc(N(c8ccccc8)c8cc9ccccc9c9ccccc89)cc7ccc6c5c34)c2)c2cc3ccccc3c3ccccc23)cc1 ; c1ccc(N(c2ccc3c(c2)OCCO3)c2ccc3c(ccc4c3oc3ccc5oc6c7ccc(N(c8ccccc8)c8ccc9c(c8)OCCO9)cc7ccc6c5c34)c2)cc1]

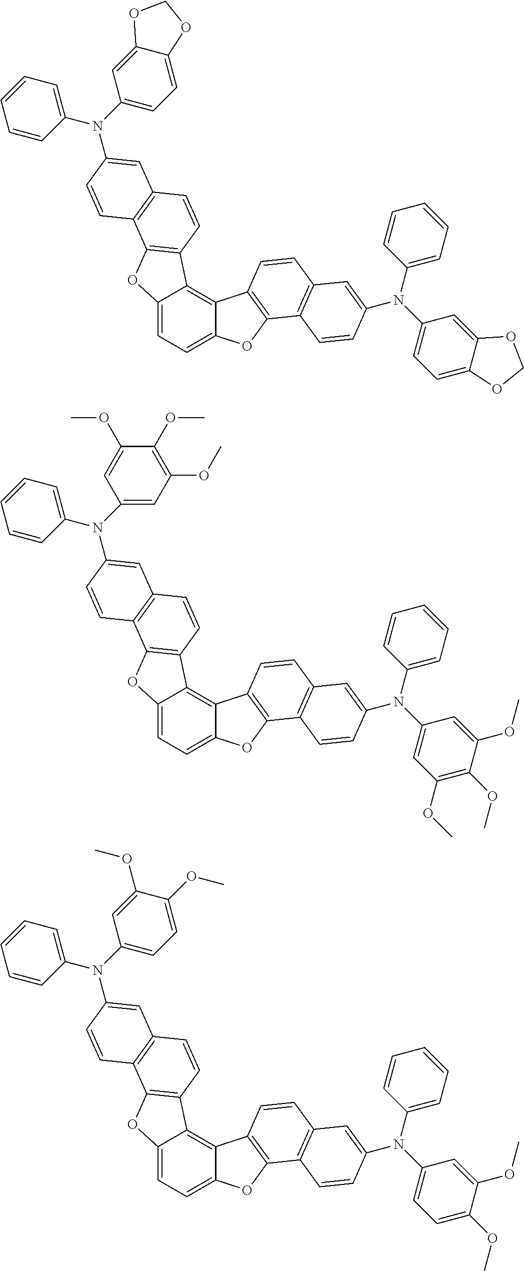 [SMILES: c1ccc(N(c2ccc3c(c2)OCO3)c2ccc3c(ccc4c3oc3ccc5oc6c7ccc(N(c8ccccc8)c8ccc9c(c8)OCO9)cc7ccc6c5c34)c2)cc1 ; COc1cc(N(c2ccccc2)c2ccc3c(ccc4c3oc3ccc5oc6c7ccc(N(c8ccccc8)c8cc(OC)c(OC)c(OC)c8)cc7ccc6c5c34)c2)cc(OC)c1OC ; COc1ccc(N(c2ccccc2)c2ccc3c(ccc4c3oc3ccc5oc6c7ccc(N(c8ccccc8)c8ccc(OC)c(OC)c8)cc7ccc6c5c34)c2)cc1OC]

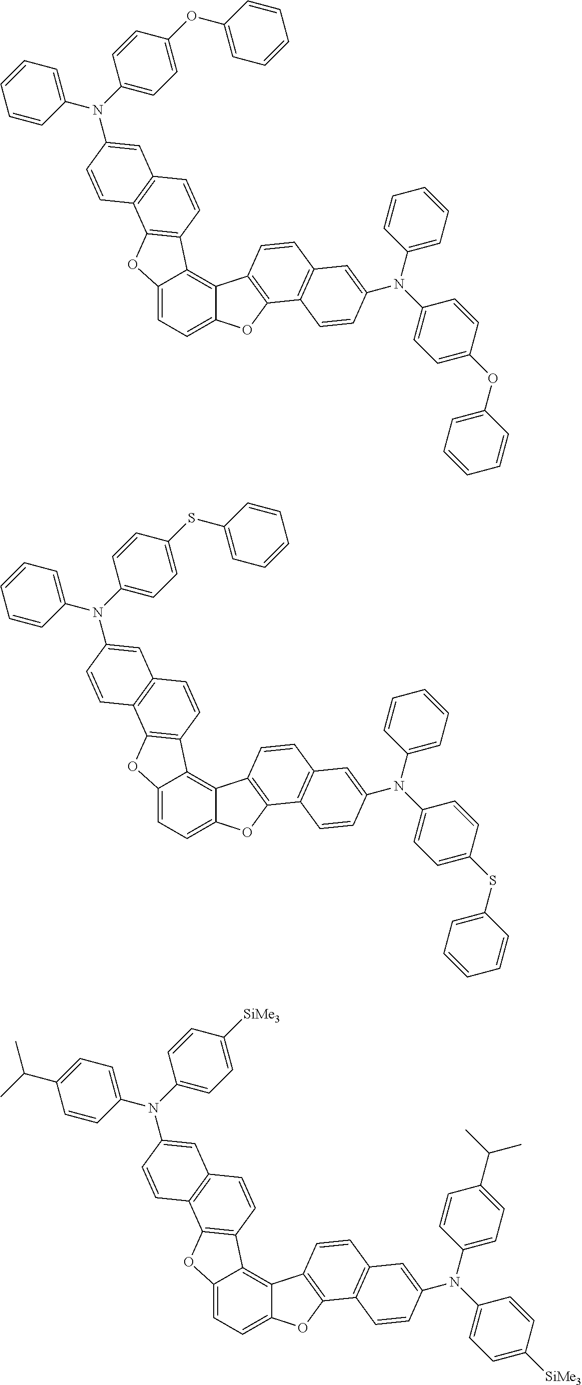 [SMILES: c1ccc(Oc2ccc(N(c3ccccc3)c3ccc4c(ccc5c4oc4ccc6oc7c8ccc(N(c9ccccc9)c9ccc(Oc%10ccccc%10)cc9)cc8ccc7c6c45)c3)cc2)cc1 ; c1ccc(Sc2ccc(N(c3ccccc3)c3ccc4c(ccc5c4oc4ccc6oc7c8ccc(N(c9ccccc9)c9ccc(Sc%10ccccc%10)cc9)cc8ccc7c6c45)c3)cc2)cc1 ; CC(C)c1ccc(N(c2ccc([Si](C)(C)C)cc2)c2ccc3c(ccc4c3oc3ccc5oc6c7ccc(N(c8ccc(C(C)C)cc8)c8ccc([Si](C)(C)C)cc8)cc7ccc6c5c34)c2)cc1]

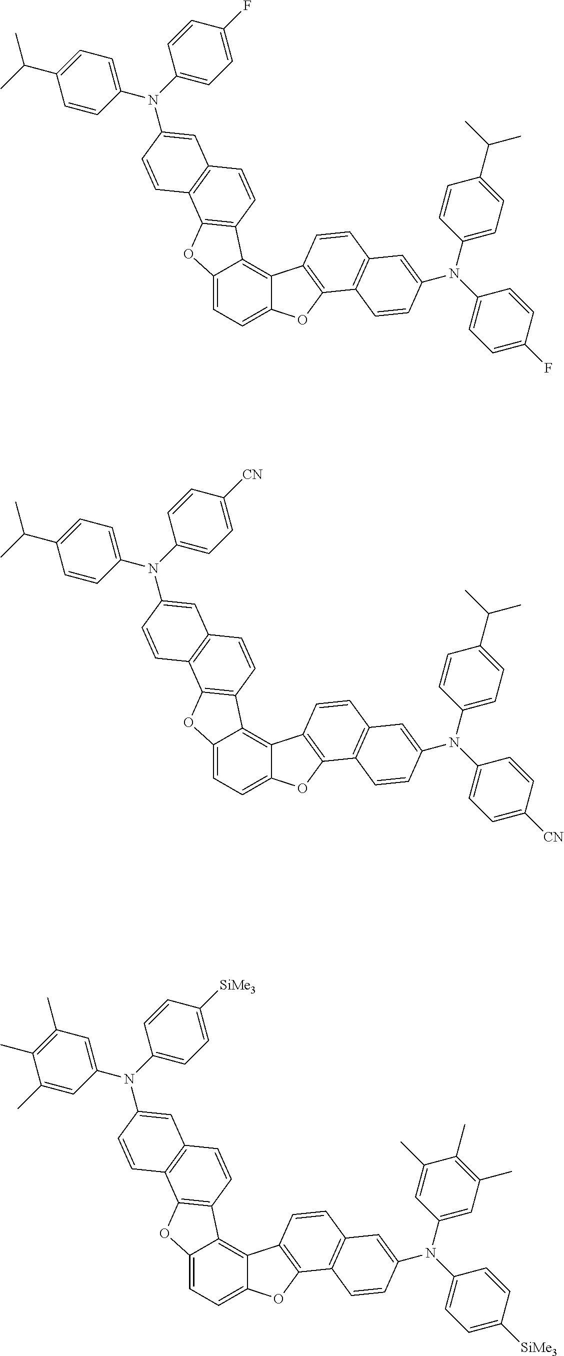 [SMILES: CC(C)c1ccc(N(c2ccc(F)cc2)c2ccc3c(ccc4c3oc3ccc5oc6c7ccc(N(c8ccc(F)cc8)c8ccc(C(C)C)cc8)cc7ccc6c5c34)c2)cc1 ; CC(C)c1ccc(N(c2ccc(C#N)cc2)c2ccc3c(ccc4c3oc3ccc5oc6c7ccc(N(c8ccc(C#N)cc8)c8ccc(C(C)C)cc8)cc7ccc6c5c34)c2)cc1 ; Cc1cc(N(c2ccc([Si](C)(C)C)cc2)c2ccc3c(ccc4c3oc3ccc5oc6c7ccc(N(c8ccc([Si](C)(C)C)cc8)c8cc(C)c(C)c(C)c8)cc7ccc6c5c34)c2)cc(C)c1C]

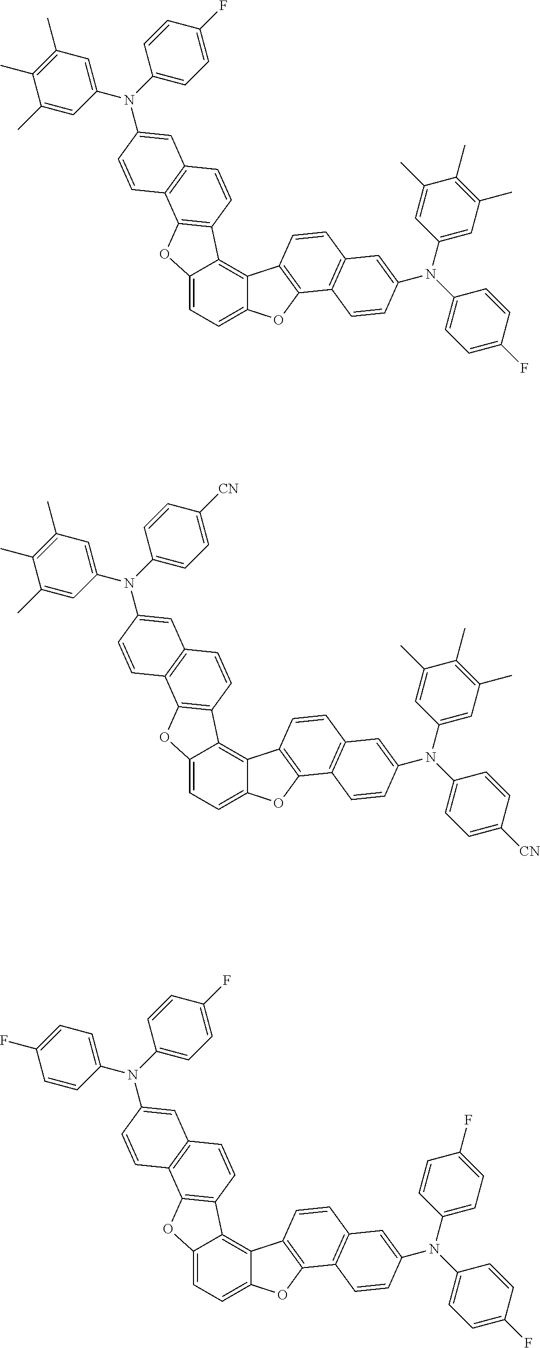 [SMILES: Cc1cc(N(c2ccc(F)cc2)c2ccc3c(ccc4c3oc3ccc5oc6c7ccc(N(c8ccc(F)cc8)c8cc(C)c(C)c(C)c8)cc7ccc6c5c34)c2)cc(C)c1C ; Cc1cc(N(c2ccc(C#N)cc2)c2ccc3c(ccc4c3oc3ccc5oc6c7ccc(N(c8ccc(C#N)cc8)c8cc(C)c(C)c(C)c8)cc7ccc6c5c34)c2)cc(C)c1C ; Fc1ccc(N(c2ccc(F)cc2)c2ccc3c(ccc4c3oc3ccc5oc6c7ccc(N(c8ccc(F)cc8)c8ccc(F)cc8)cc7ccc6c5c34)c2)cc1]

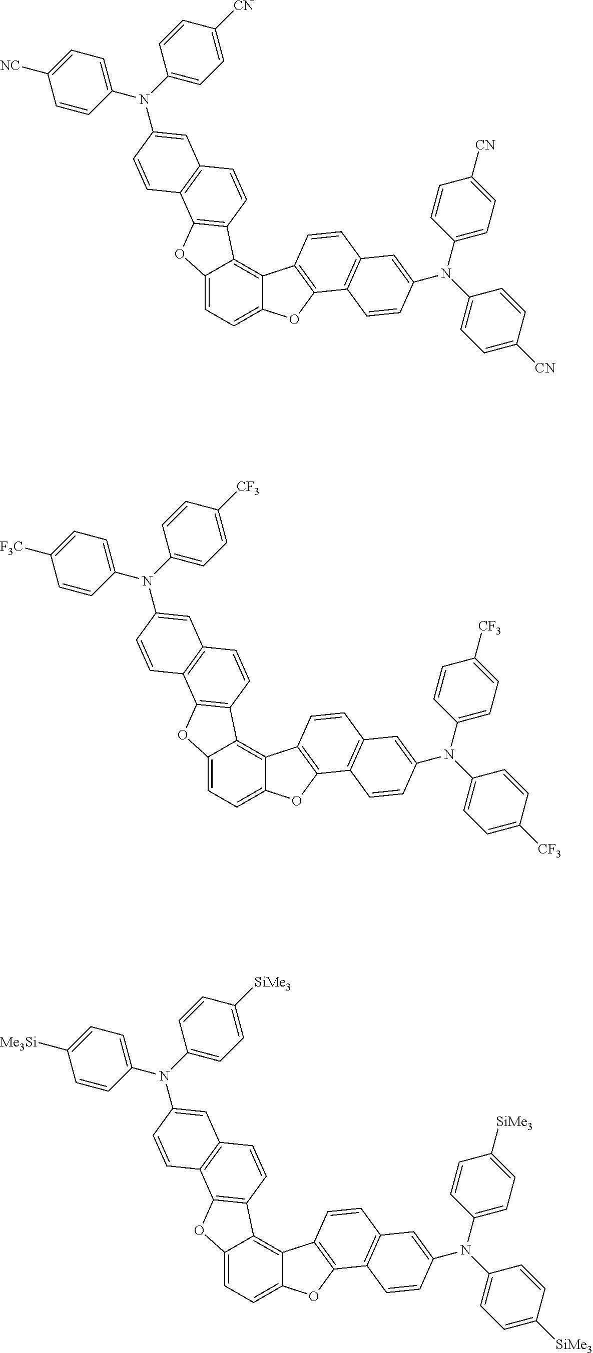 [SMILES: N#Cc1ccc(N(c2ccc(C#N)cc2)c2ccc3c(ccc4c3oc3ccc5oc6c7ccc(N(c8ccc(C#N)cc8)c8ccc(C#N)cc8)cc7ccc6c5c34)c2)cc1 ; FC(F)(F)c1ccc(N(c2ccc(C(F)(F)F)cc2)c2ccc3c(ccc4c3oc3ccc5oc6c7ccc(N(c8ccc(C(F)(F)F)cc8)c8ccc(C(F)(F)F)cc8)cc7ccc6c5c34)c2)cc1 ; C[Si](C)(C)c1ccc(N(c2ccc([Si](C)(C)C)cc2)c2ccc3c(ccc4c3oc3ccc5oc6c7ccc(N(c8ccc([Si](C)(C)C)cc8)c8ccc([Si](C)(C)C)cc8)cc7ccc6c5c34)c2)cc1]

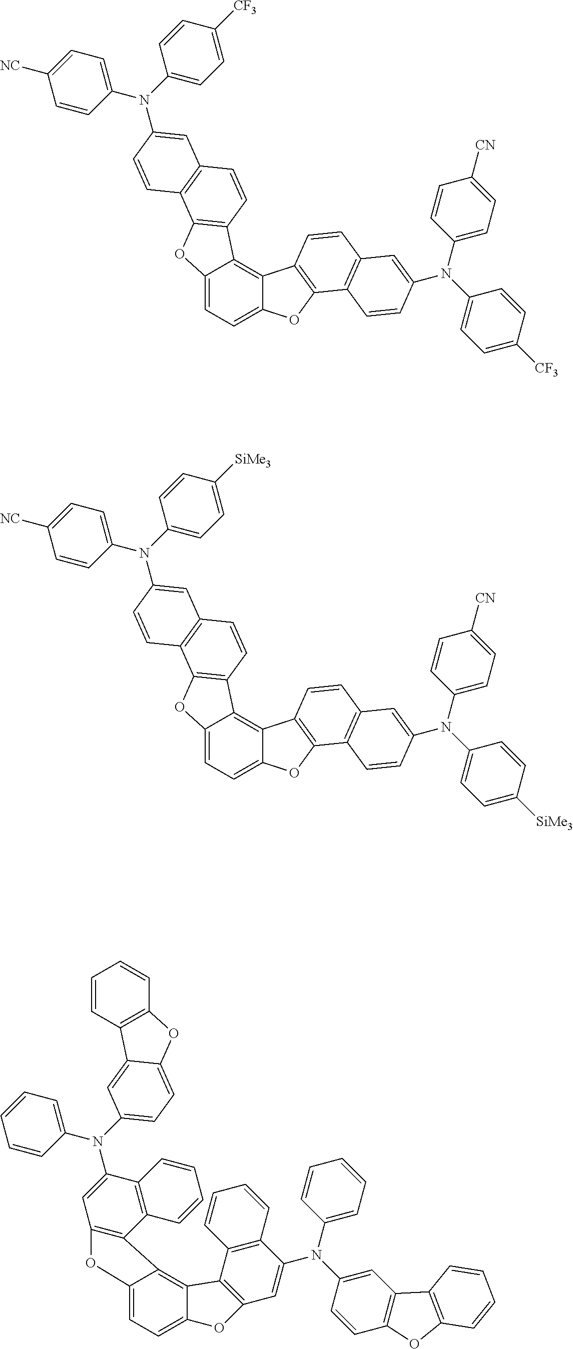 [SMILES: N#Cc1ccc(N(c2ccc(C(F)(F)F)cc2)c2ccc3c(ccc4c3oc3ccc5oc6c7ccc(N(c8ccc(C#N)cc8)c8ccc(C(F)(F)F)cc8)cc7ccc6c5c34)c2)cc1 ; C[Si](C)(C)c1ccc(N(c2ccc(C#N)cc2)c2ccc3c(ccc4c3oc3ccc5oc6c7ccc(N(c8ccc(C#N)cc8)c8ccc([Si](C)(C)C)cc8)cc7ccc6c5c34)c2)cc1 ; c1ccc(N(c2ccc3oc4ccccc4c3c2)c2cc3oc4ccc5oc6cc(N(c7ccccc7)c7ccc8oc9ccccc9c8c7)c7ccccc7c6c5c4c3c3ccccc23)cc1]

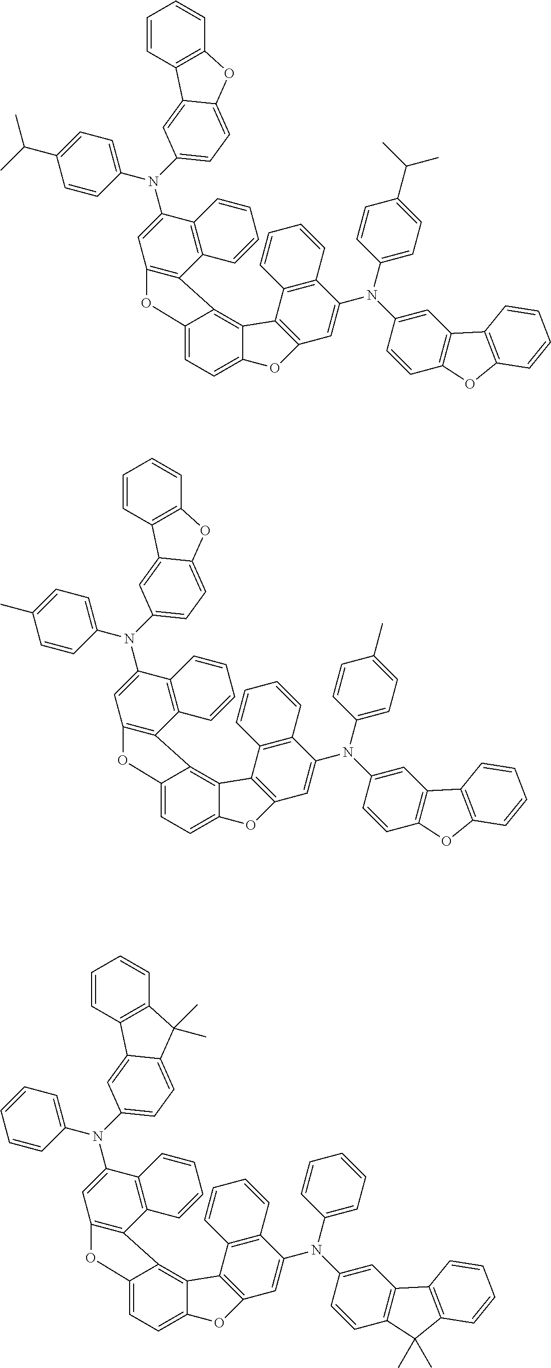 [SMILES: CC(C)c1ccc(N(c2ccc3oc4ccccc4c3c2)c2cc3oc4ccc5oc6cc(N(c7ccc(C(C)C)cc7)c7ccc8oc9ccccc9c8c7)c7ccccc7c6c5c4c3c3ccccc23)cc1 ; Cc1ccc(N(c2ccc3oc4ccccc4c3c2)c2cc3oc4ccc5oc6cc(N(c7ccc(C)cc7)c7ccc8oc9ccccc9c8c7)c7ccccc7c6c5c4c3c3ccccc23)cc1 ; CC1(C)c2ccccc2-c2cc(N(c3ccccc3)c3cc4oc5ccc6oc7cc(N(c8ccccc8)c8ccc9c(c8)-c8ccccc8C9(C)C)c8ccccc8c7c6c5c4c4ccccc34)ccc21]

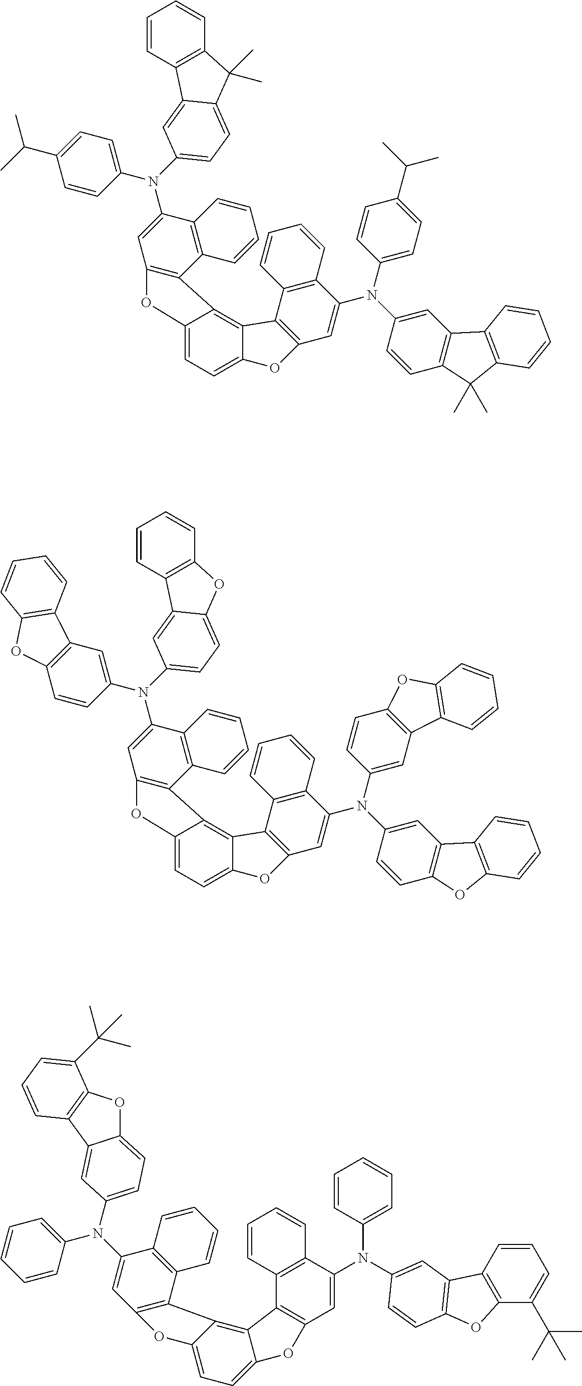 [SMILES: CC(C)c1ccc(N(c2ccc3c(c2)-c2ccccc2C3(C)C)c2cc3oc4ccc5oc6cc(N(c7ccc(C(C)C)cc7)c7ccc8c(c7)-c7ccccc7C8(C)C)c7ccccc7c6c5c4c3c3ccccc23)cc1 ; c1ccc2c(c1)oc1ccc(N(c3ccc4oc5ccccc5c4c3)c3cc4oc5ccc6oc7cc(N(c8ccc9oc%10ccccc%10c9c8)c8ccc9oc%10ccccc%10c9c8)c8ccccc8c7c6c5c4c4ccccc34)cc12 ; CC(C)(C)c1cccc2c1oc1ccc(N(c3ccccc3)c3cc4oc5ccc6oc7cc(N(c8ccccc8)c8ccc9oc%10c(C(C)(C)C)cccc%10c9c8)c8ccccc8c7c6c5c4c4ccccc34)cc12]

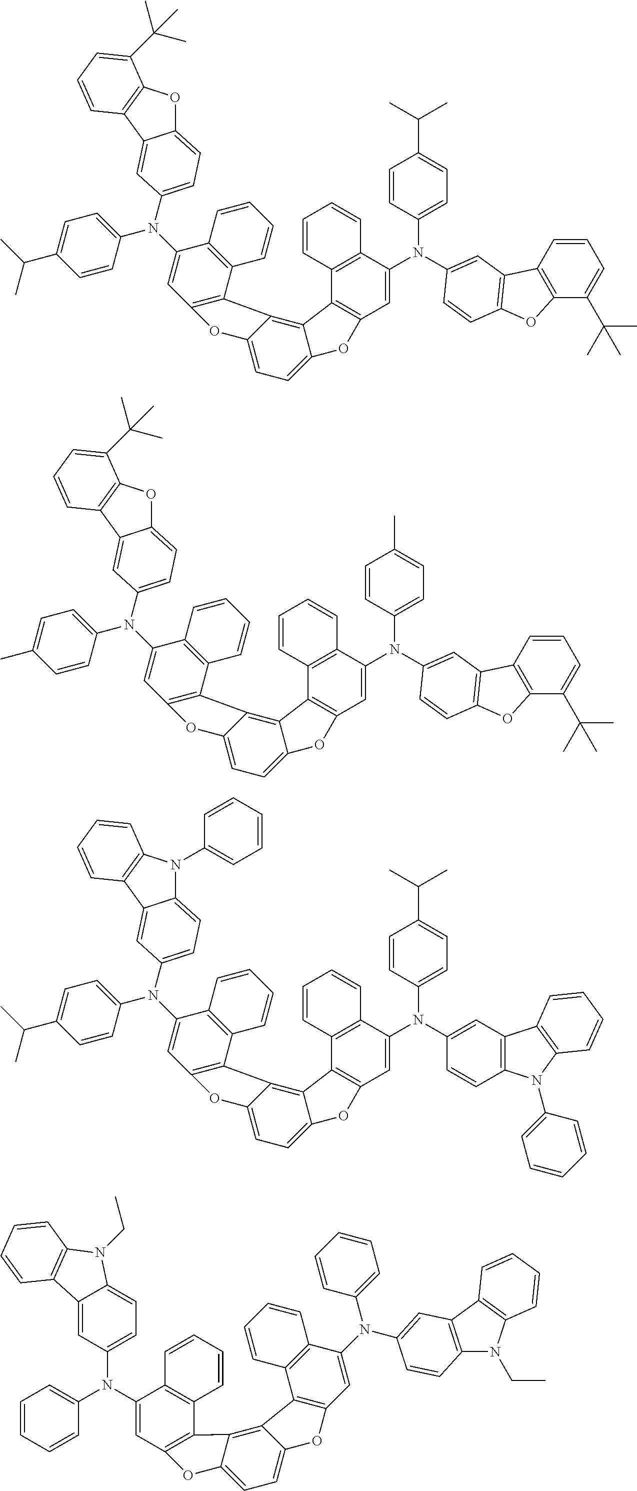

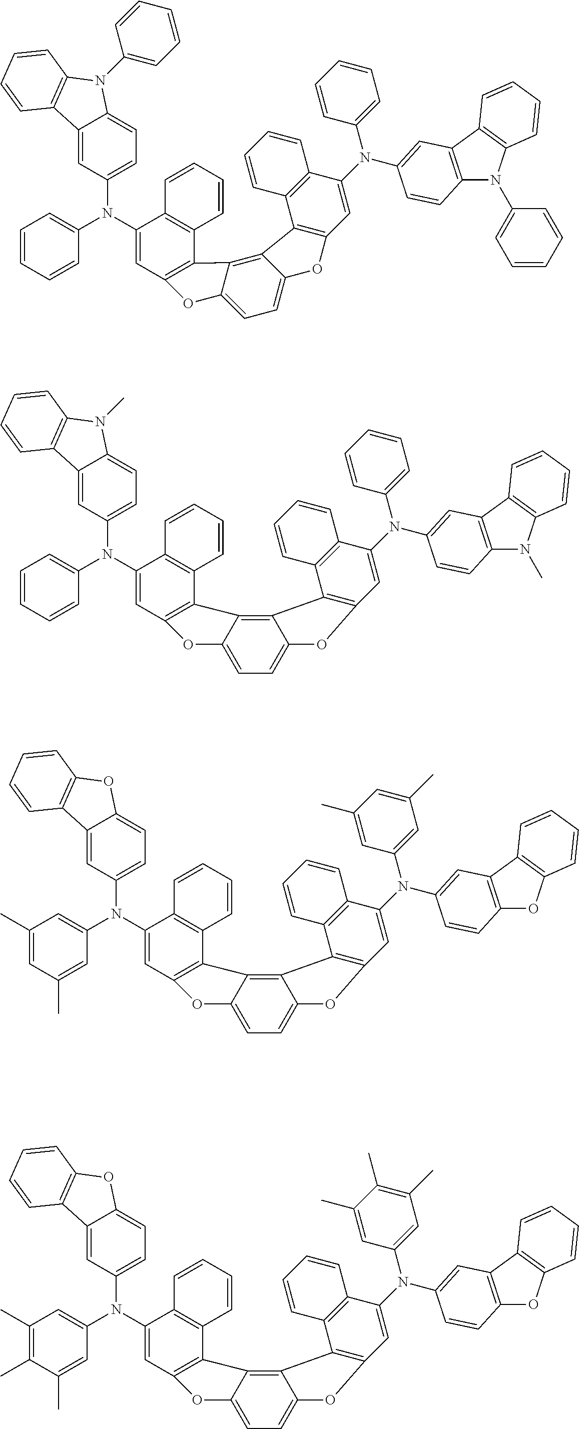 [SMILES: c1ccc(N(c2ccc3c(c2)c2ccccc2n3-c2ccccc2)c2cc3oc4ccc5oc6cc(N(c7ccccc7)c7ccc8c(c7)c7ccccc7n8-c7ccccc7)c7ccccc7c6c5c4c3c3ccccc23)cc1 ; Cn1c2ccccc2c2cc(N(c3ccccc3)c3cc4oc5ccc6oc7cc(N(c8ccccc8)c8ccc9c(c8)c8ccccc8n9C)c8ccccc8c7c6c5c4c4ccccc34)ccc21 ; Cc1cc(C)cc(N(c2ccc3oc4ccccc4c3c2)c2cc3oc4ccc5oc6cc(N(c7cc(C)cc(C)c7)c7ccc8oc9ccccc9c8c7)c7ccccc7c6c5c4c3c3ccccc23)c1 ; Cc1cc(N(c2ccc3oc4ccccc4c3c2)c2cc3oc4ccc5oc6cc(N(c7cc(C)c(C)c(C)c7)c7ccc8oc9ccccc9c8c7)c7ccccc7c6c5c4c3c3ccccc23)cc(C)c1C]

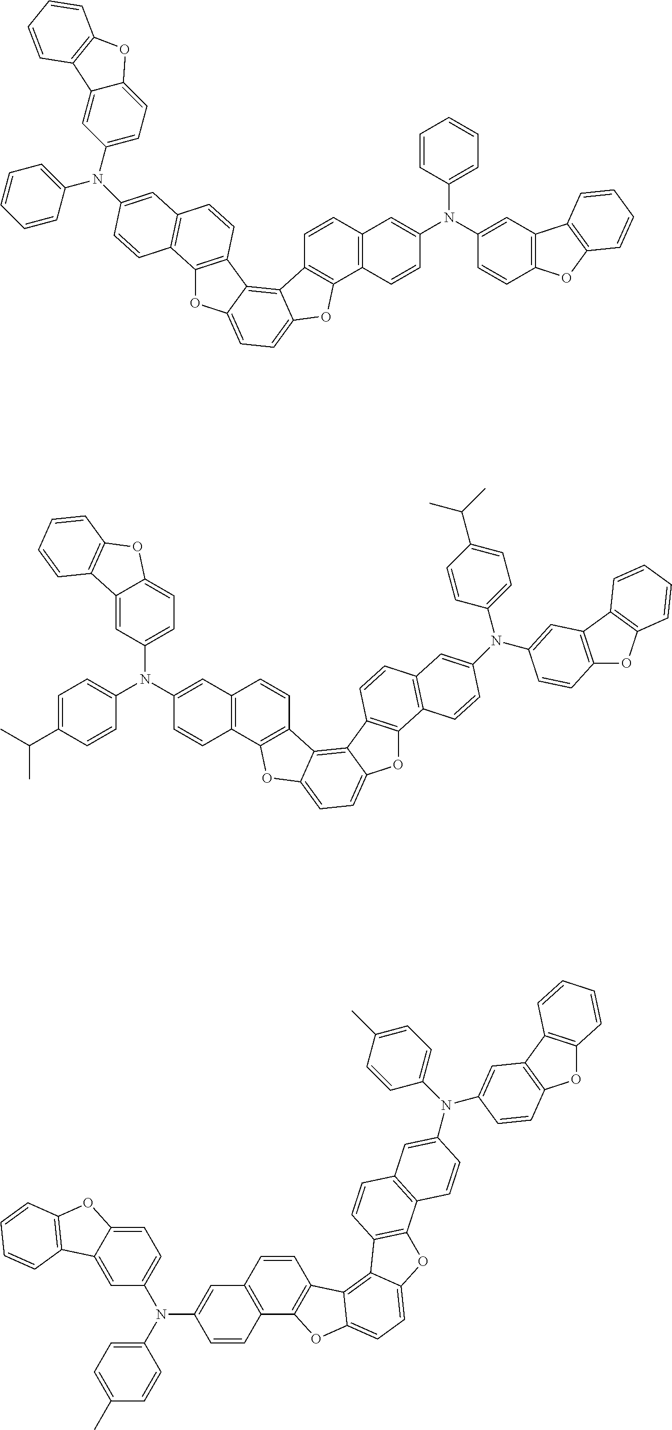 [SMILES: c1ccc(N(c2ccc3c(ccc4c3oc3ccc5oc6c7ccc(N(c8ccccc8)c8ccc9oc%10ccccc%10c9c8)cc7ccc6c5c34)c2)c2ccc3oc4ccccc4c3c2)cc1 ; CC(C)c1ccc(N(c2ccc3c(ccc4c3oc3ccc5oc6c7ccc(N(c8ccc(C(C)C)cc8)c8ccc9oc%10ccccc%10c9c8)cc7ccc6c5c34)c2)c2ccc3oc4ccccc4c3c2)cc1 ; Cc1ccc(N(c2ccc3c(ccc4c3oc3ccc5oc6c7ccc(N(c8ccc(C)cc8)c8ccc9oc%10ccccc%10c9c8)cc7ccc6c5c34)c2)c2ccc3oc4ccccc4c3c2)cc1]

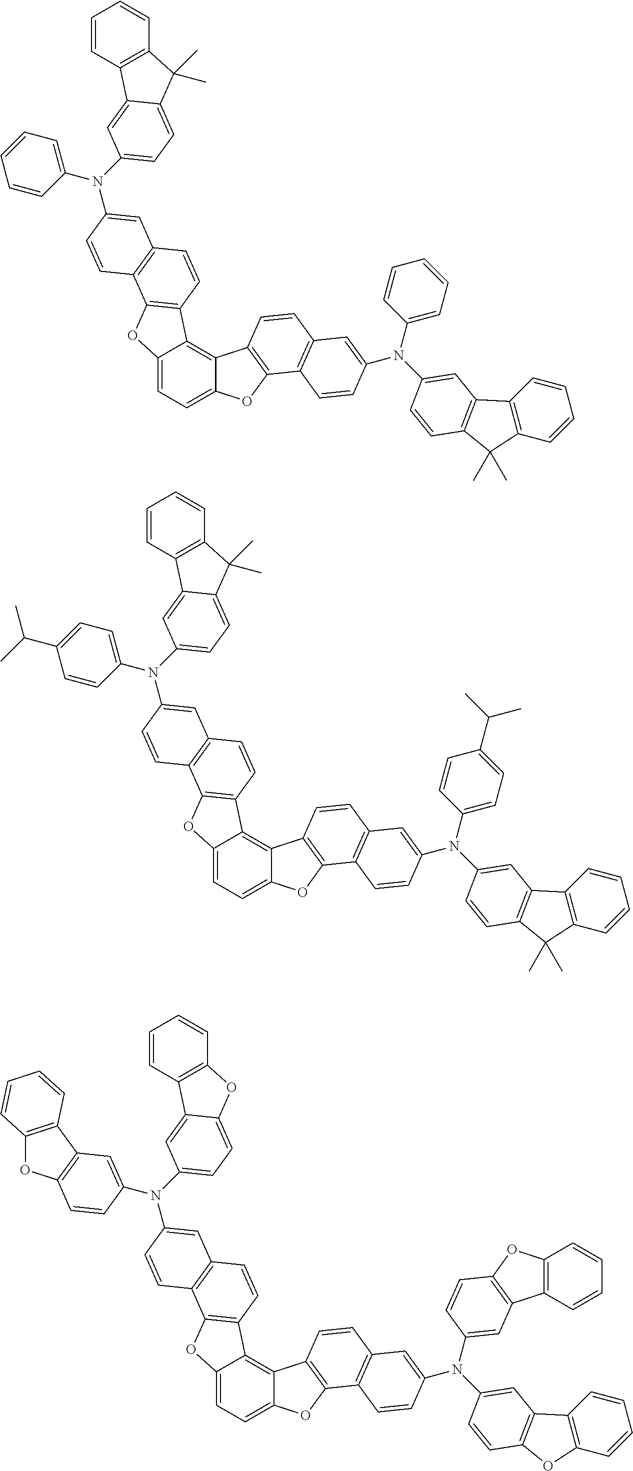 [SMILES: CC1(C)c2ccccc2-c2cc(N(c3ccccc3)c3ccc4c(ccc5c4oc4ccc6oc7c8ccc(N(c9ccccc9)c9ccc%10c(c9)-c9ccccc9C%10(C)C)cc8ccc7c6c45)c3)ccc21 ; CC(C)c1ccc(N(c2ccc3c(c2)-c2ccccc2C3(C)C)c2ccc3c(ccc4c3oc3ccc5oc6c7ccc(N(c8ccc(C(C)C)cc8)c8ccc9c(c8)-c8ccccc8C9(C)C)cc7ccc6c5c34)c2)cc1 ; c1ccc2c(c1)oc1ccc(N(c3ccc4c(ccc5c4oc4ccc6oc7c8ccc(N(c9ccc%10oc%11ccccc%11c%10c9)c9ccc%10oc%11ccccc%11c%10c9)cc8ccc7c6c45)c3)c3ccc4oc5ccccc5c4c3)cc12]

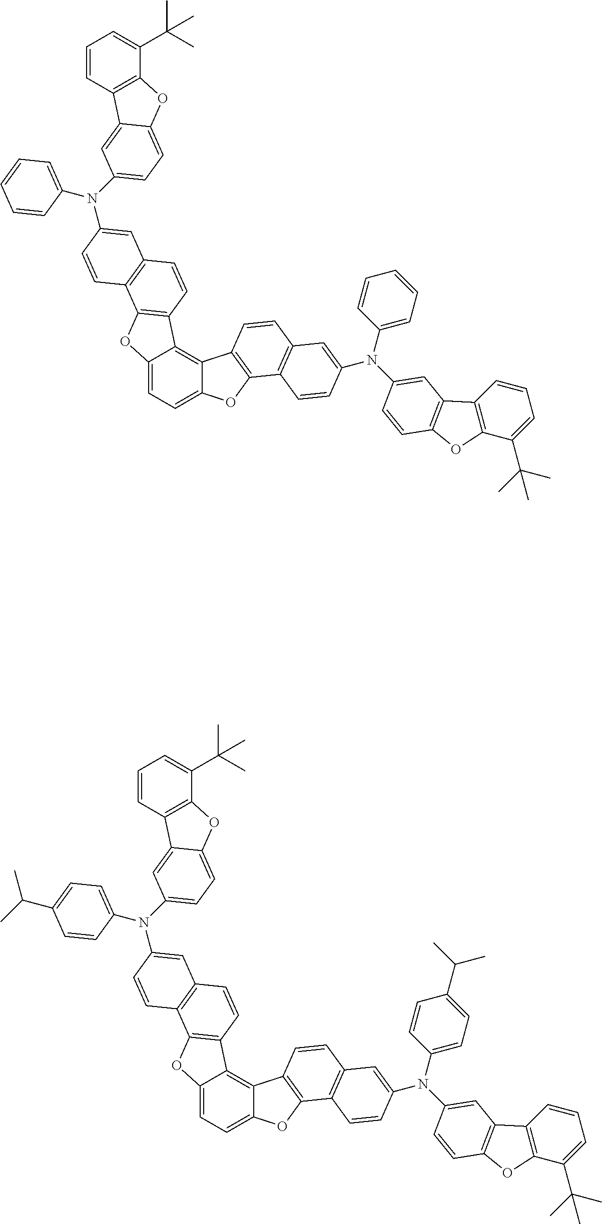

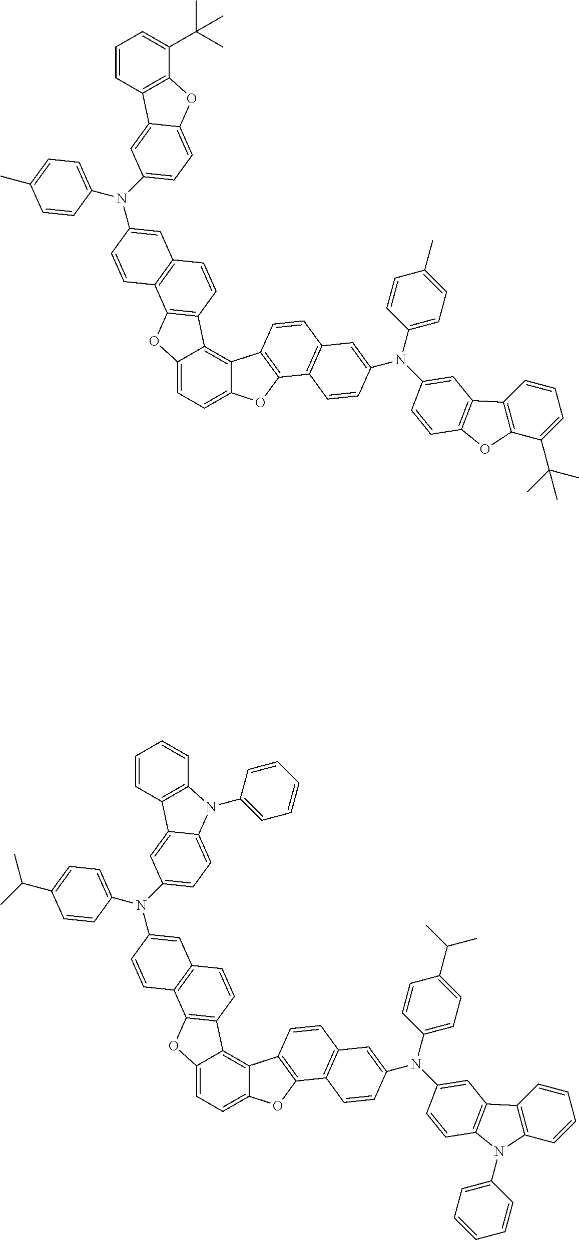

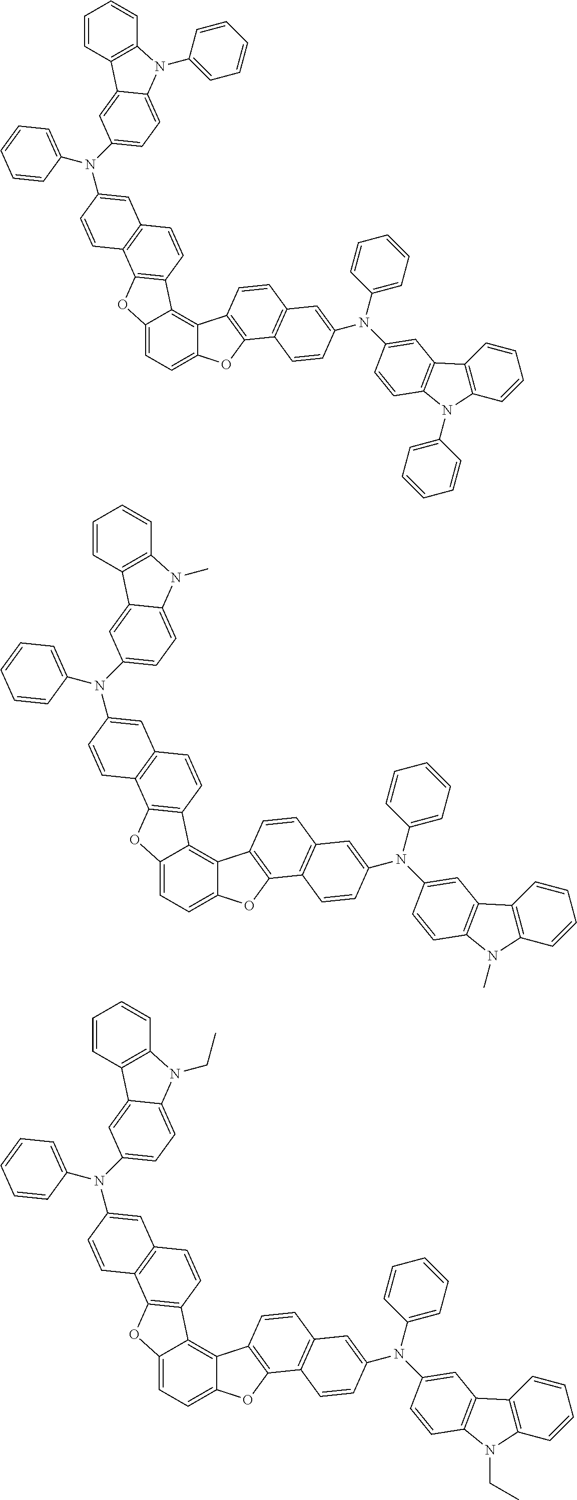 [SMILES: c1ccc(N(c2ccc3c(ccc4c3oc3ccc5oc6c7ccc(N(c8ccccc8)c8ccc9c(c8)c8ccccc8n9-c8ccccc8)cc7ccc6c5c34)c2)c2ccc3c(c2)c2ccccc2n3-c2ccccc2)cc1 ; Cn1c2ccccc2c2cc(N(c3ccccc3)c3ccc4c(ccc5c4oc4ccc6oc7c8ccc(N(c9ccccc9)c9ccc%10c(c9)c9ccccc9n%10C)cc8ccc7c6c45)c3)ccc21 ; CCn1c2ccccc2c2cc(N(c3ccccc3)c3ccc4c(ccc5c4oc4ccc6oc7c8ccc(N(c9ccccc9)c9ccc%10c(c9)c9ccccc9n%10CC)cc8ccc7c6c45)c3)ccc21]

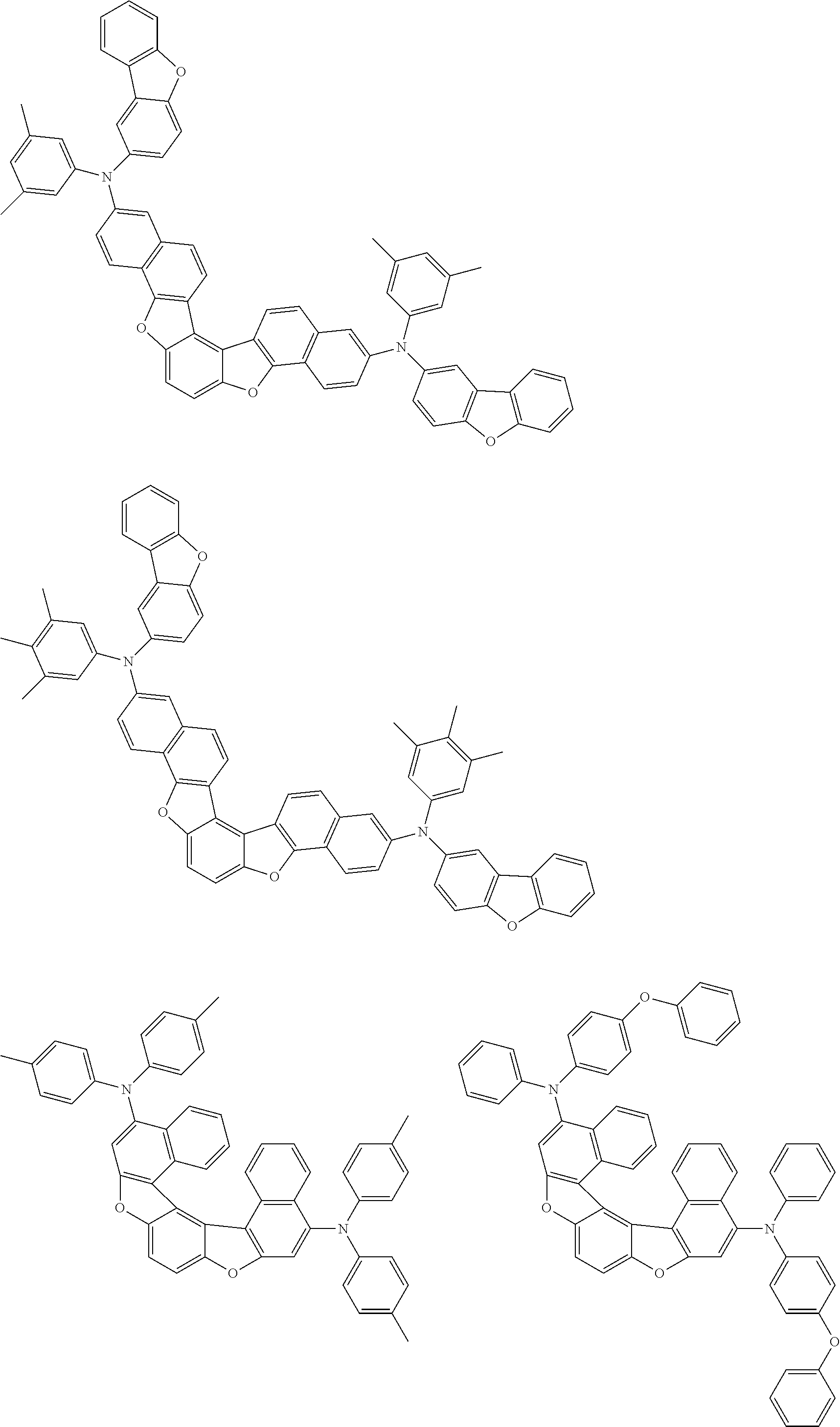

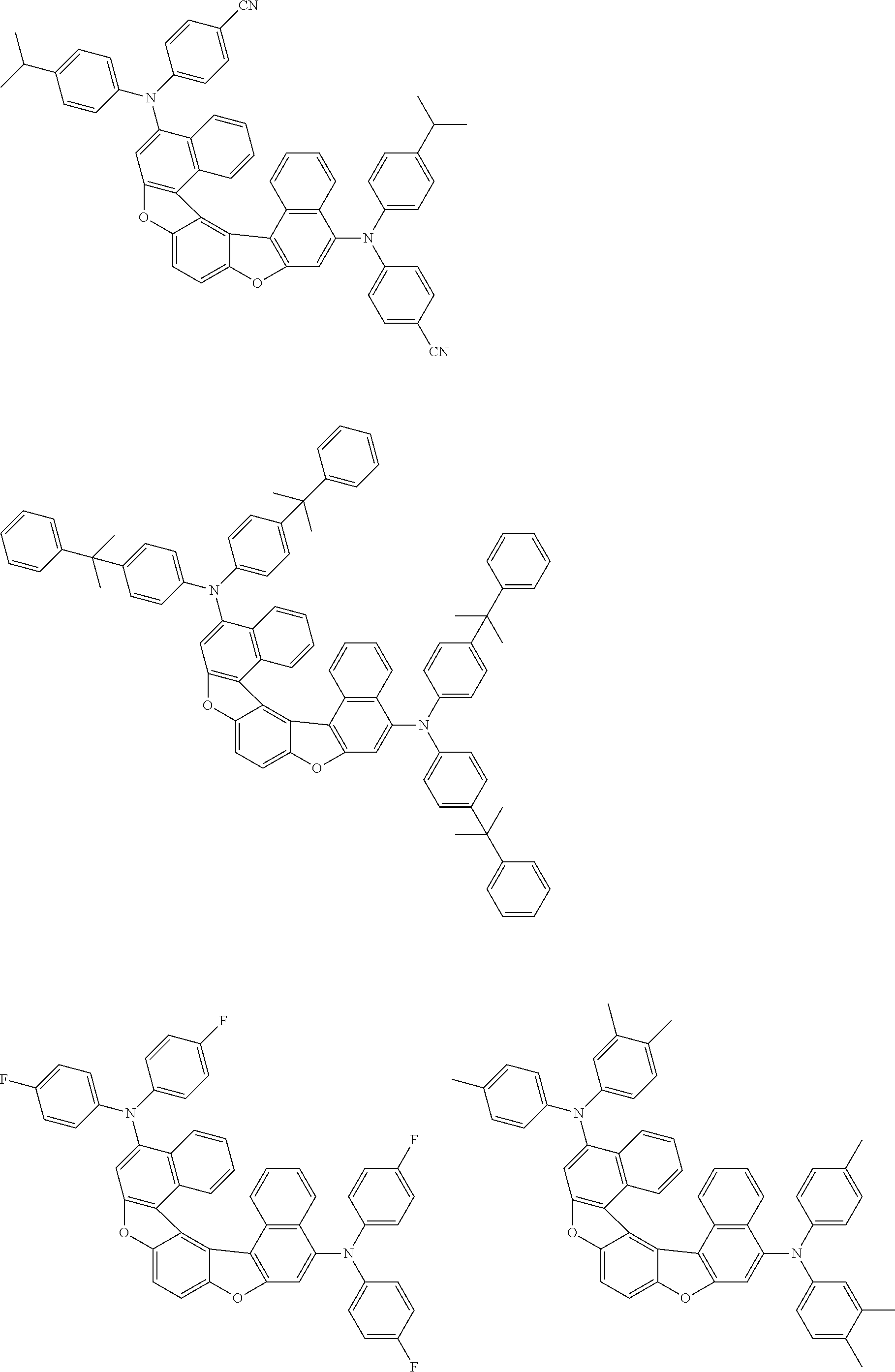

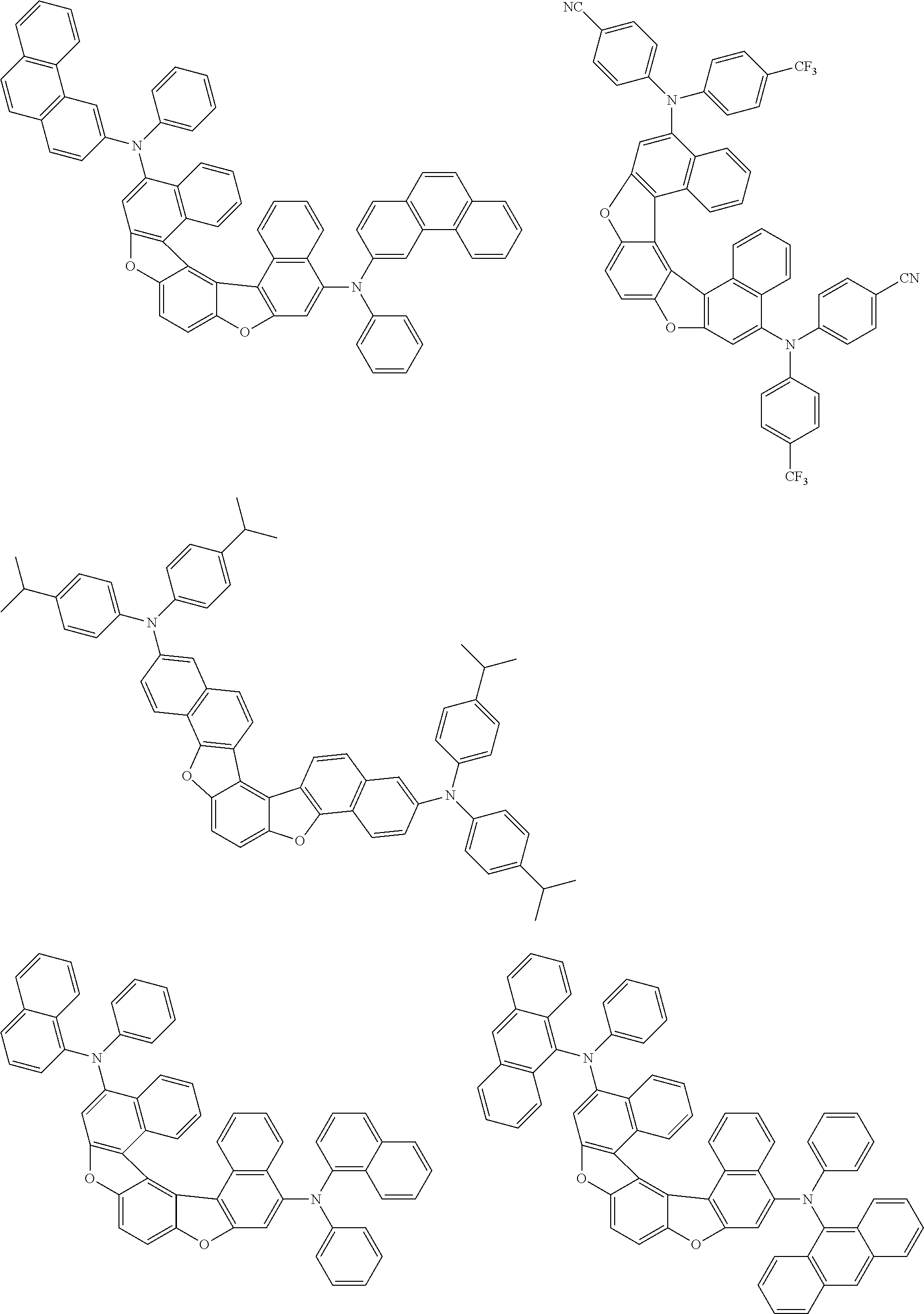

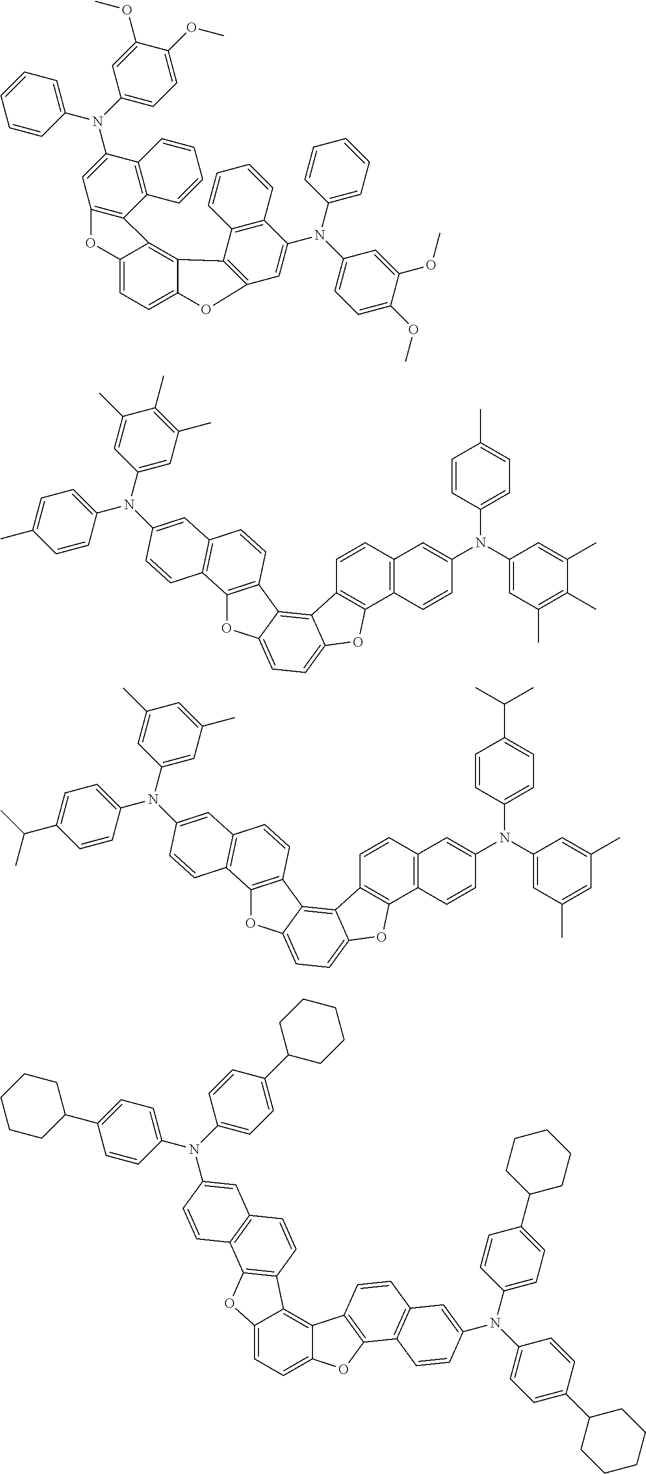

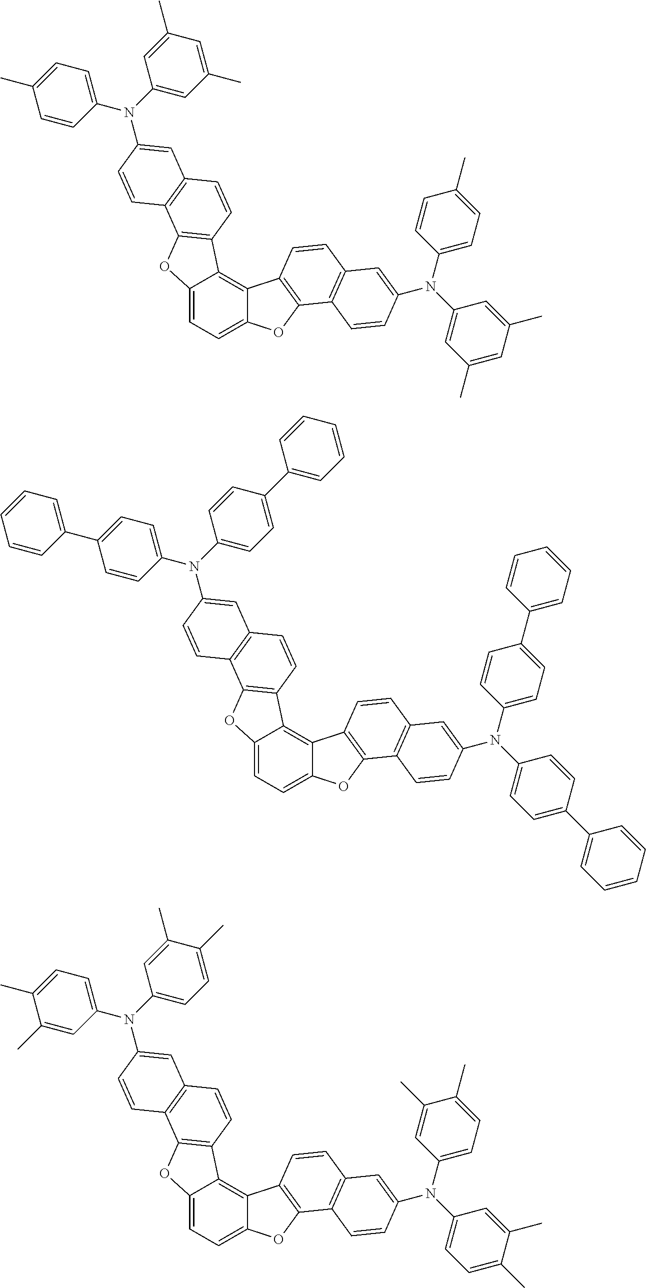

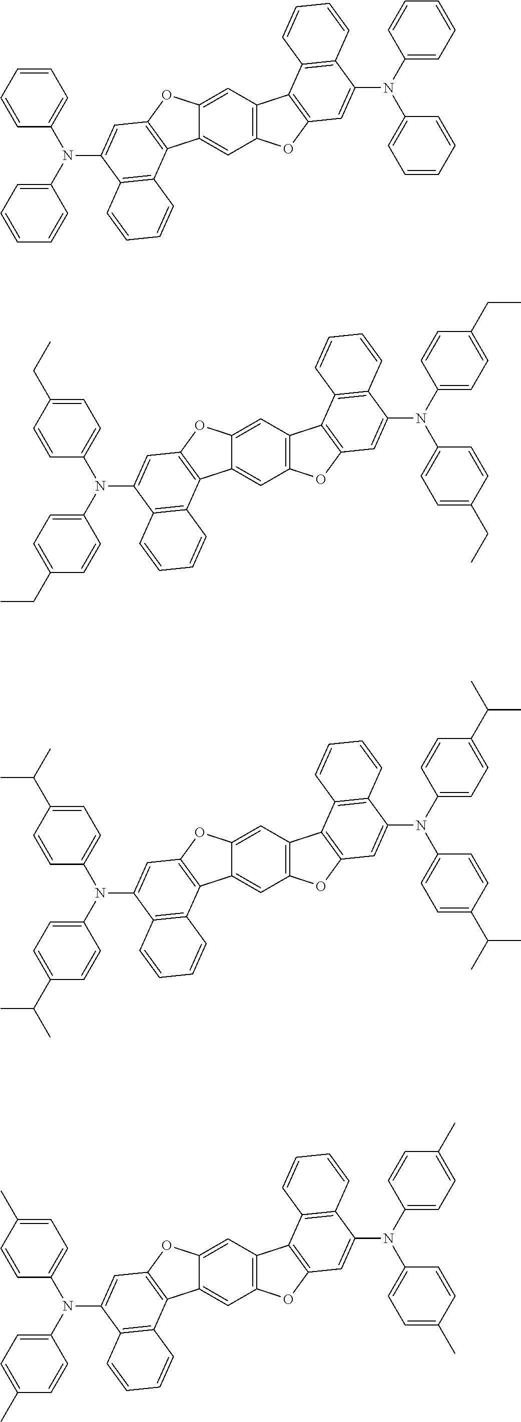

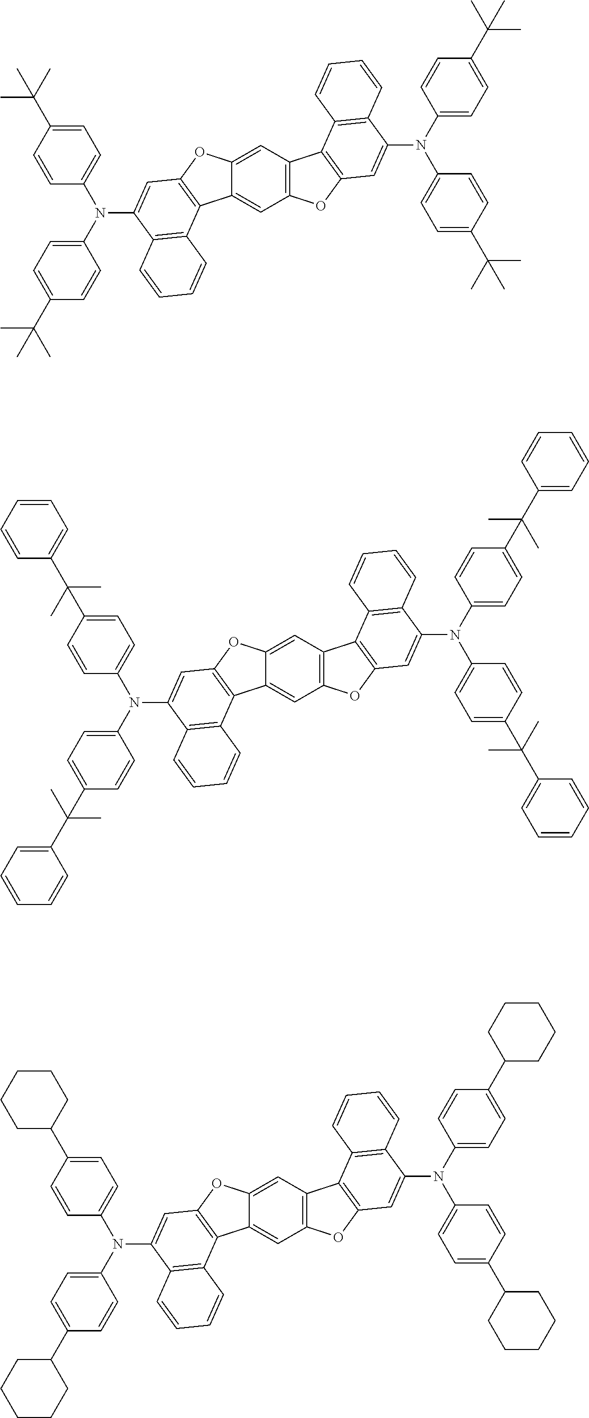 [SMILES: CC(C)(C)c1ccc(N(c2ccc(C(C)(C)C)cc2)c2cc3oc4cc5c(cc4c3c3ccccc23)oc2cc(N(c3ccc(C(C)(C)C)cc3)c3ccc(C(C)(C)C)cc3)c3ccccc3c25)cc1 ; CC(C)(c1ccccc1)c1ccc(N(c2ccc(C(C)(C)c3ccccc3)cc2)c2cc3oc4cc5c(cc4c3c3ccccc23)oc2cc(N(c3ccc(C(C)(C)c4ccccc4)cc3)c3ccc(C(C)(C)c4ccccc4)cc3)c3ccccc3c25)cc1 ; c1ccc2c(c1)c(N(c1ccc(C3CCCCC3)cc1)c1ccc(C3CCCCC3)cc1)cc1oc3cc4c(cc3c12)oc1cc(N(c2ccc(C3CCCCC3)cc2)c2ccc(C3CCCCC3)cc2)c2ccccc2c14]

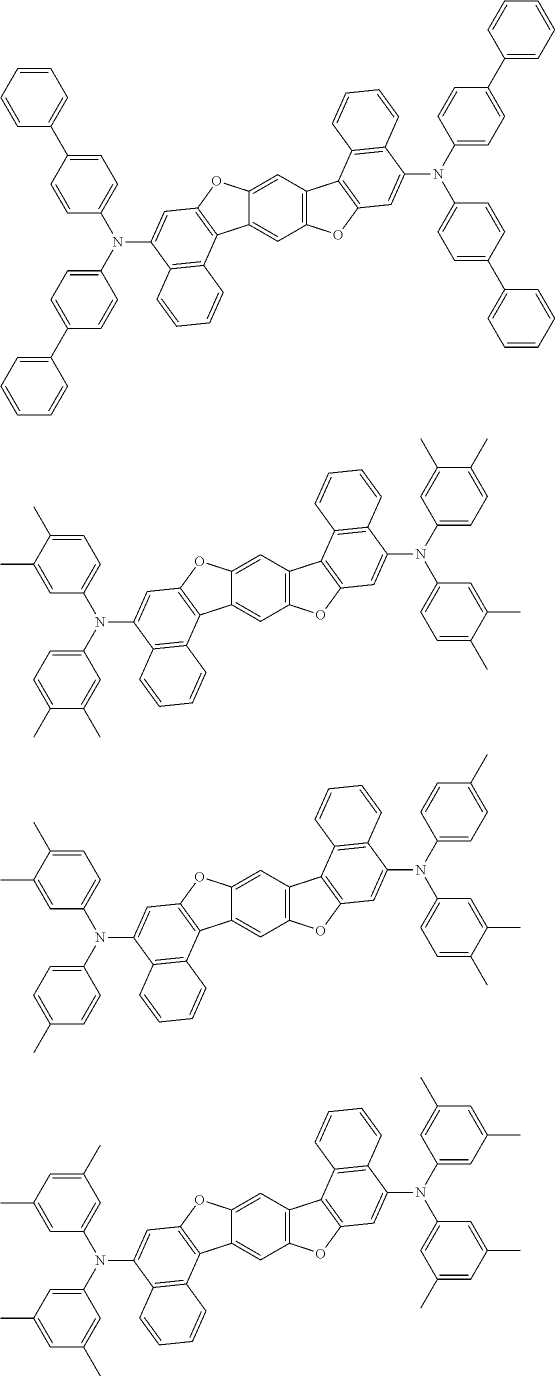

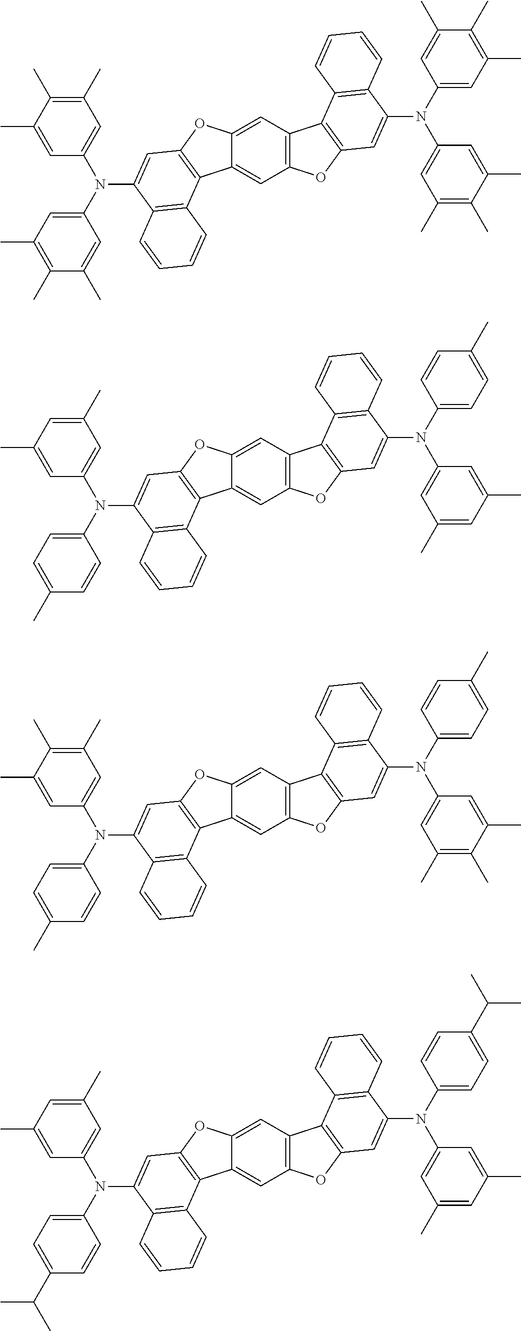

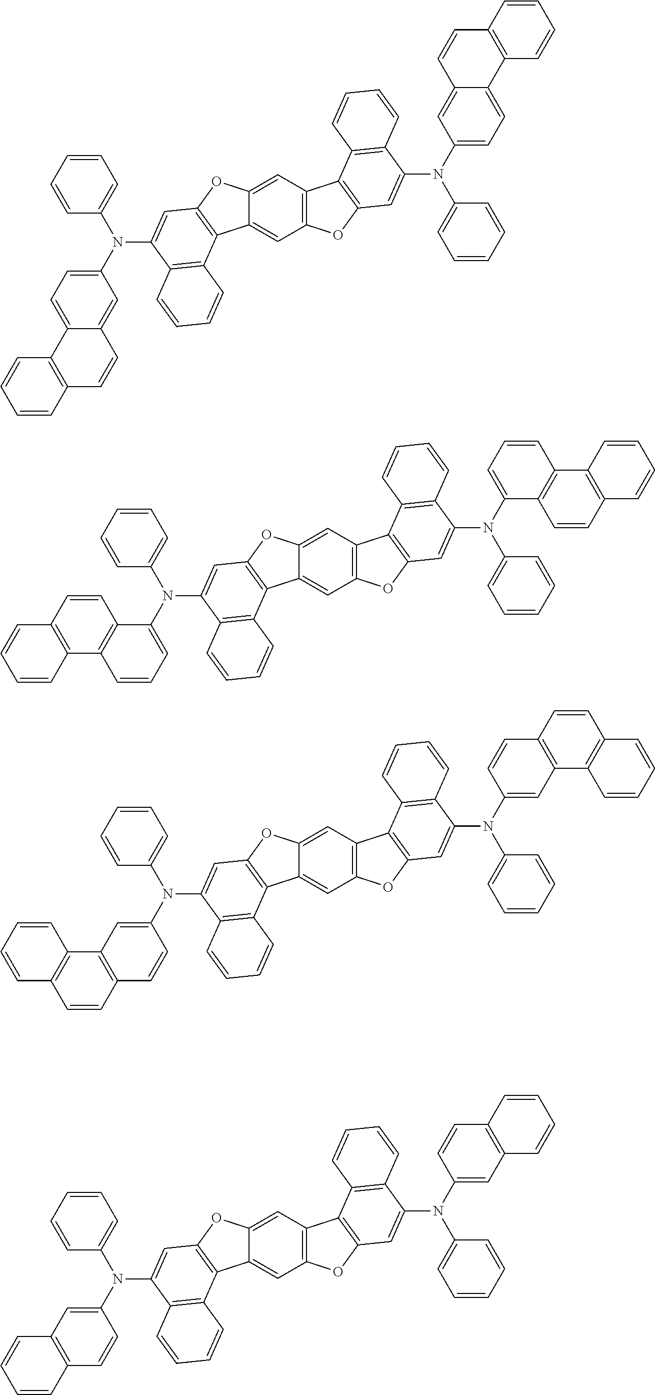 [SMILES: c1ccc(N(c2ccc3c(ccc4ccccc43)c2)c2cc3oc4cc5c(cc4c3c3ccccc23)oc2cc(N(c3ccccc3)c3ccc4c(ccc6ccccc64)c3)c3ccccc3c25)cc1 ; c1ccc(N(c2cccc3c2ccc2ccccc23)c2cc3oc4cc5c(cc4c3c3ccccc23)oc2cc(N(c3ccccc3)c3cccc4c3ccc3ccccc34)c3ccccc3c25)cc1 ; c1ccc(N(c2ccc3ccc4ccccc4c3c2)c2cc3oc4cc5c(cc4c3c3ccccc23)oc2cc(N(c3ccccc3)c3ccc4ccc6ccccc6c4c3)c3ccccc3c25)cc1 ; c1ccc(N(c2ccc3ccccc3c2)c2cc3oc4cc5c(cc4c3c3ccccc23)oc2cc(N(c3ccccc3)c3ccc4ccccc4c3)c3ccccc3c25)cc1]

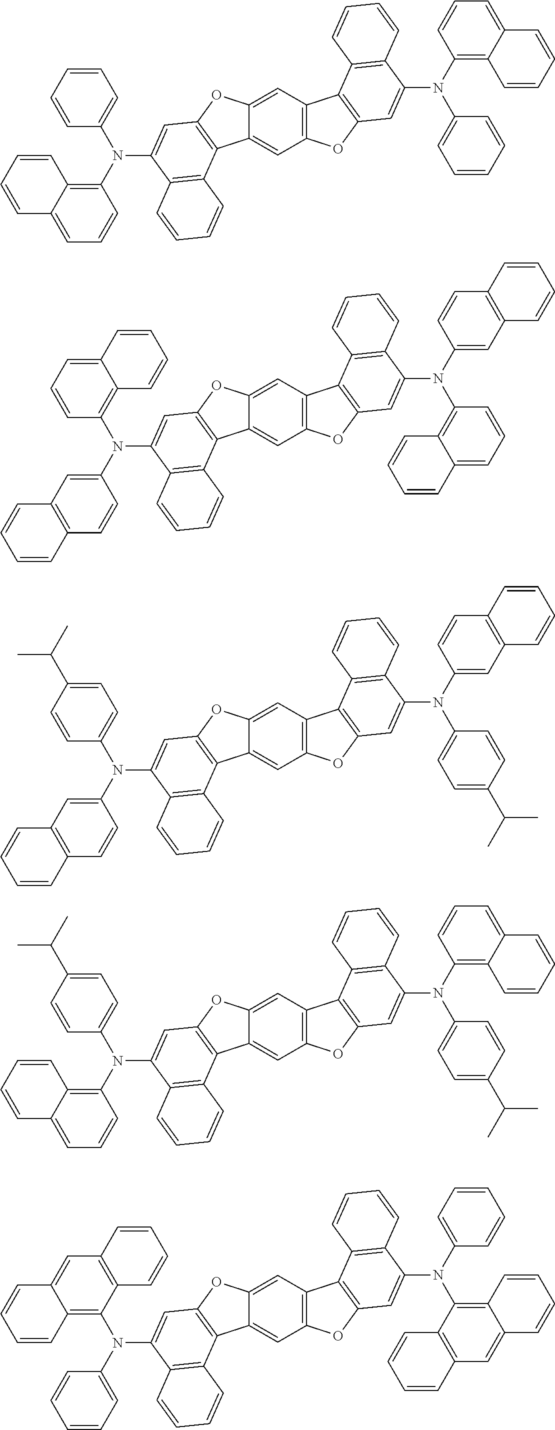

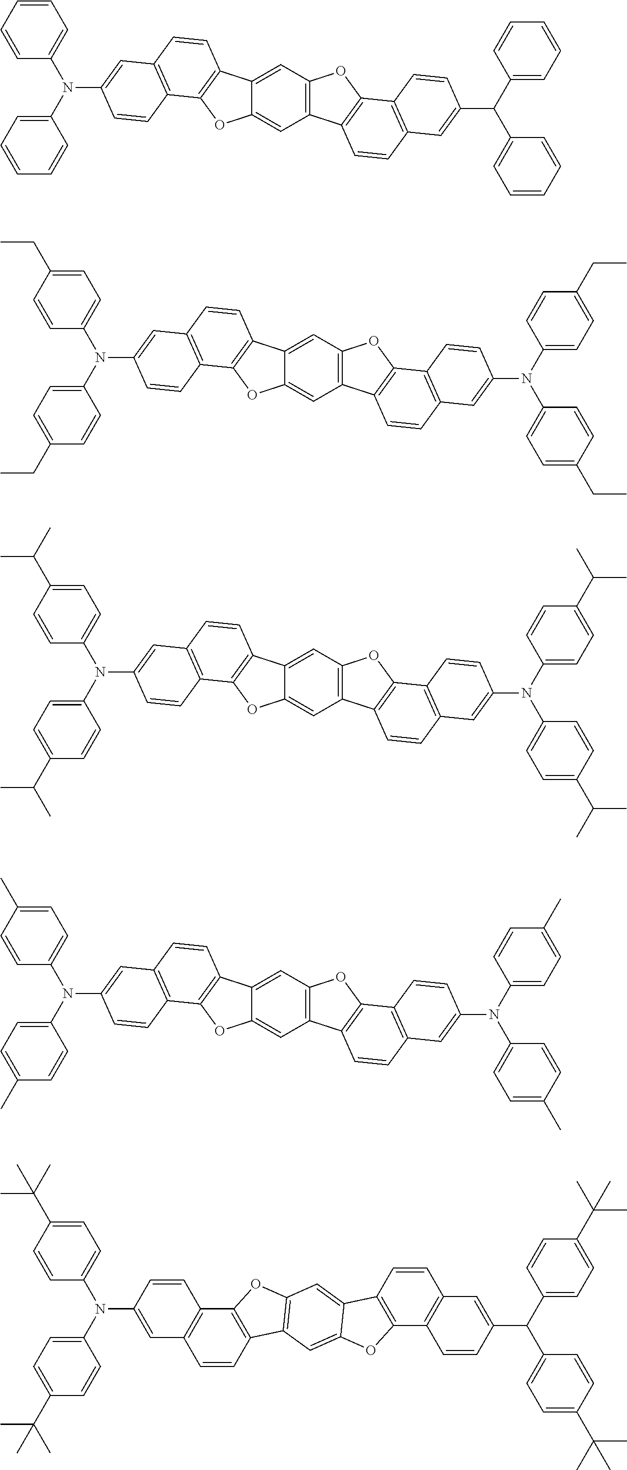

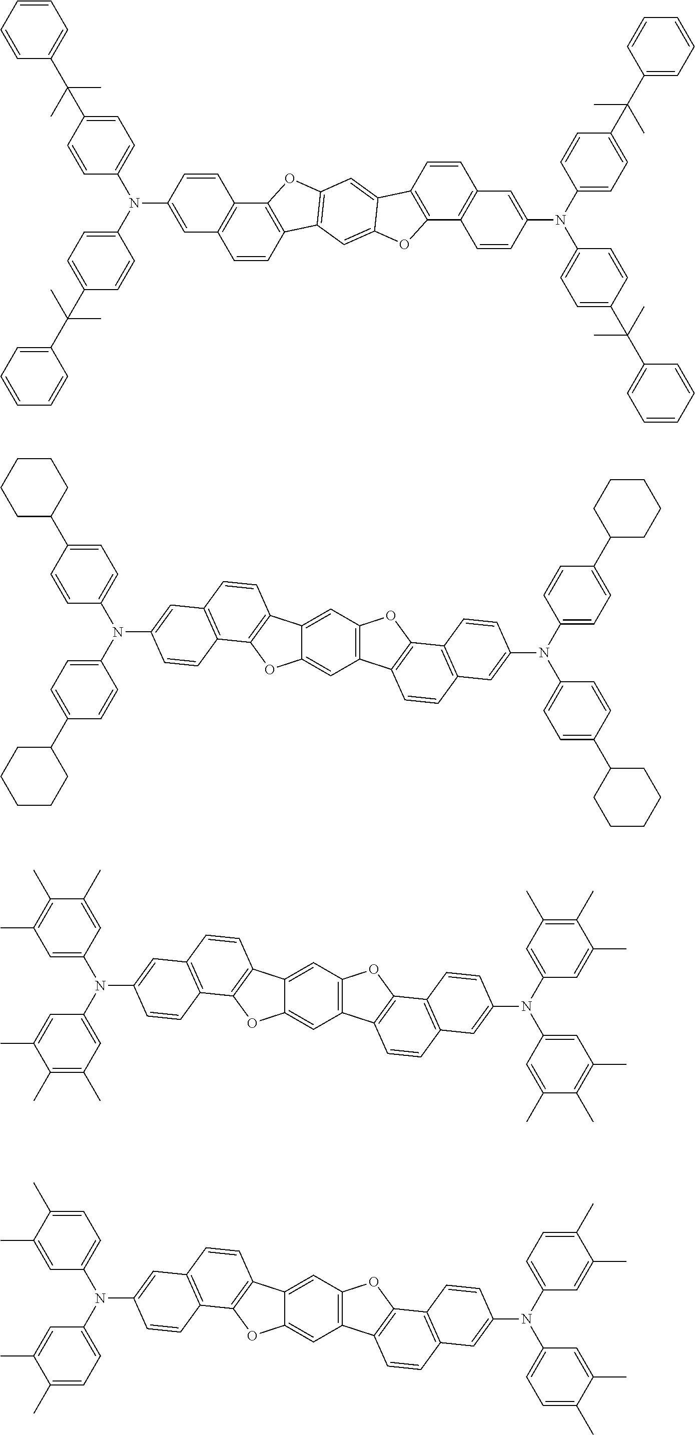

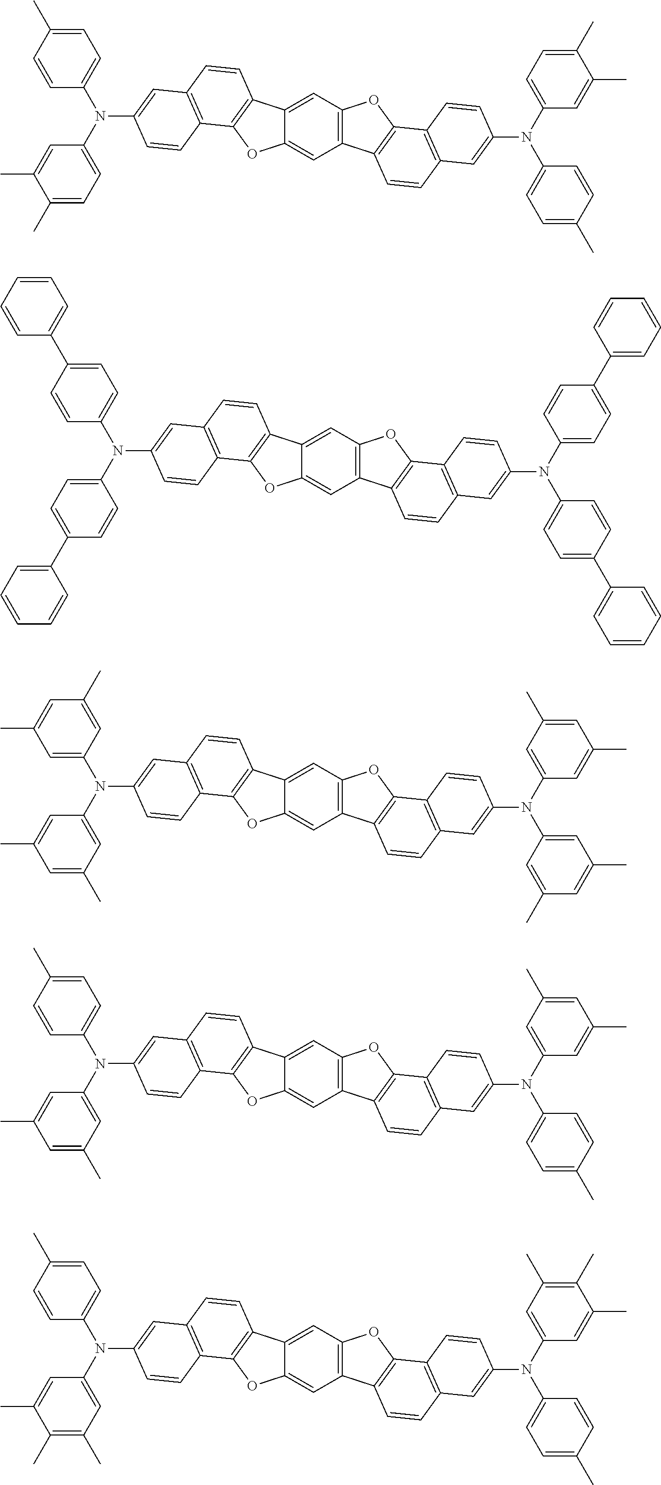

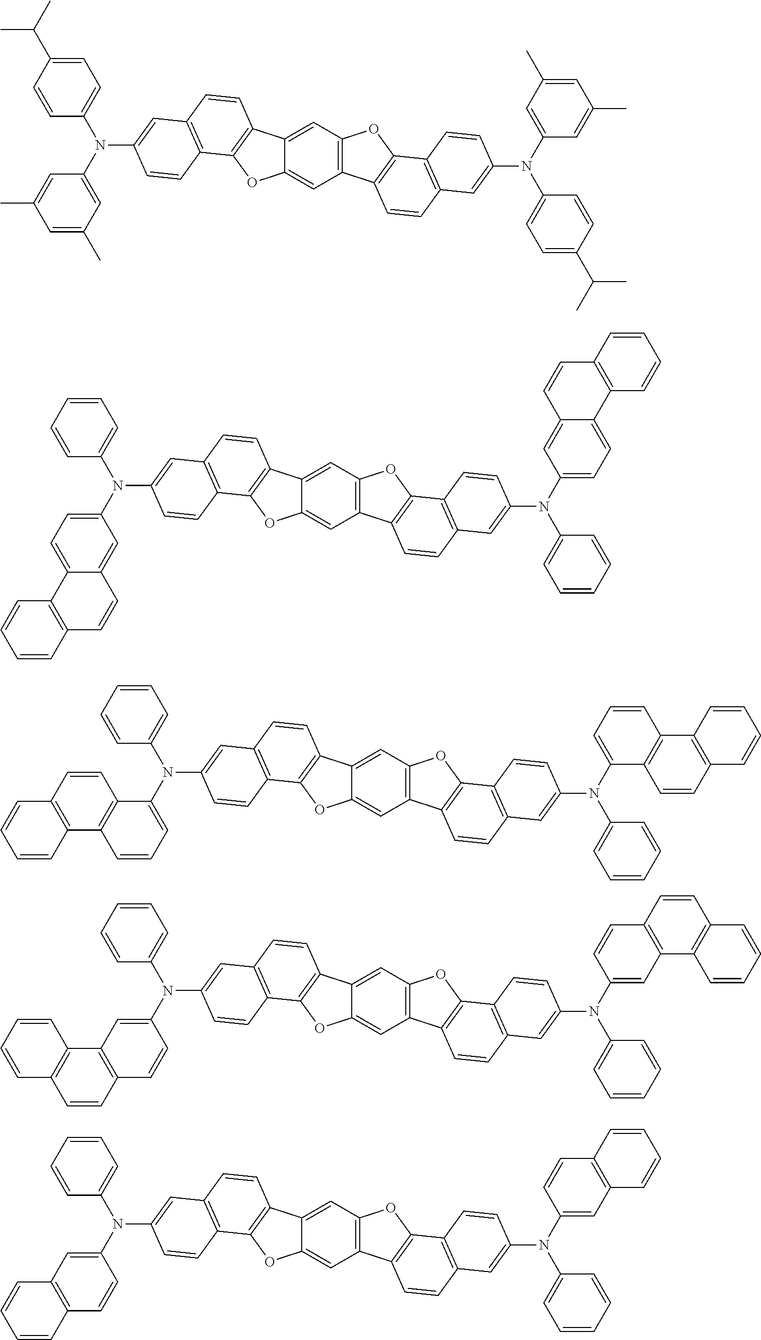

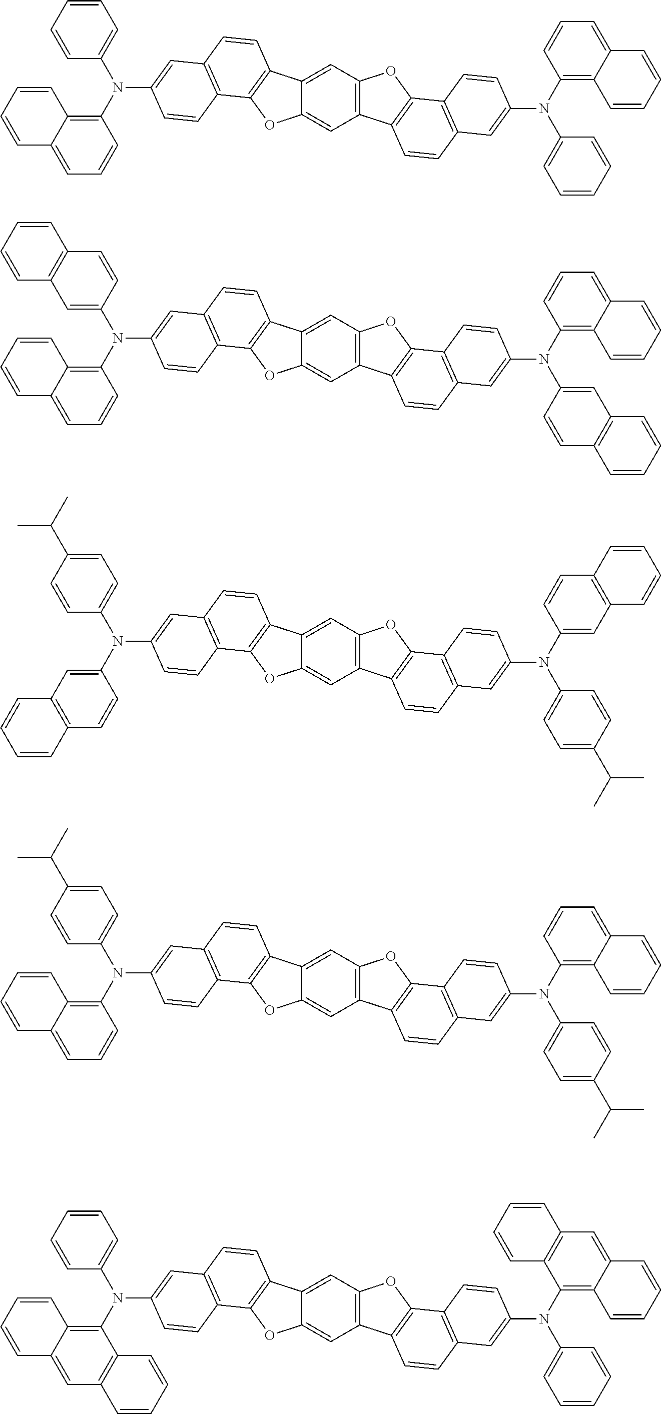

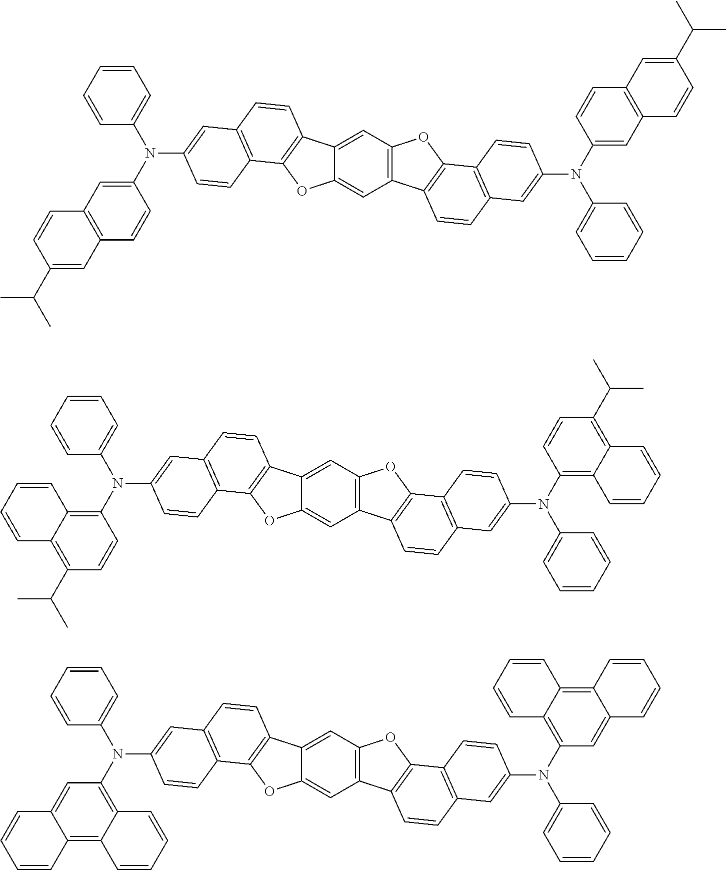

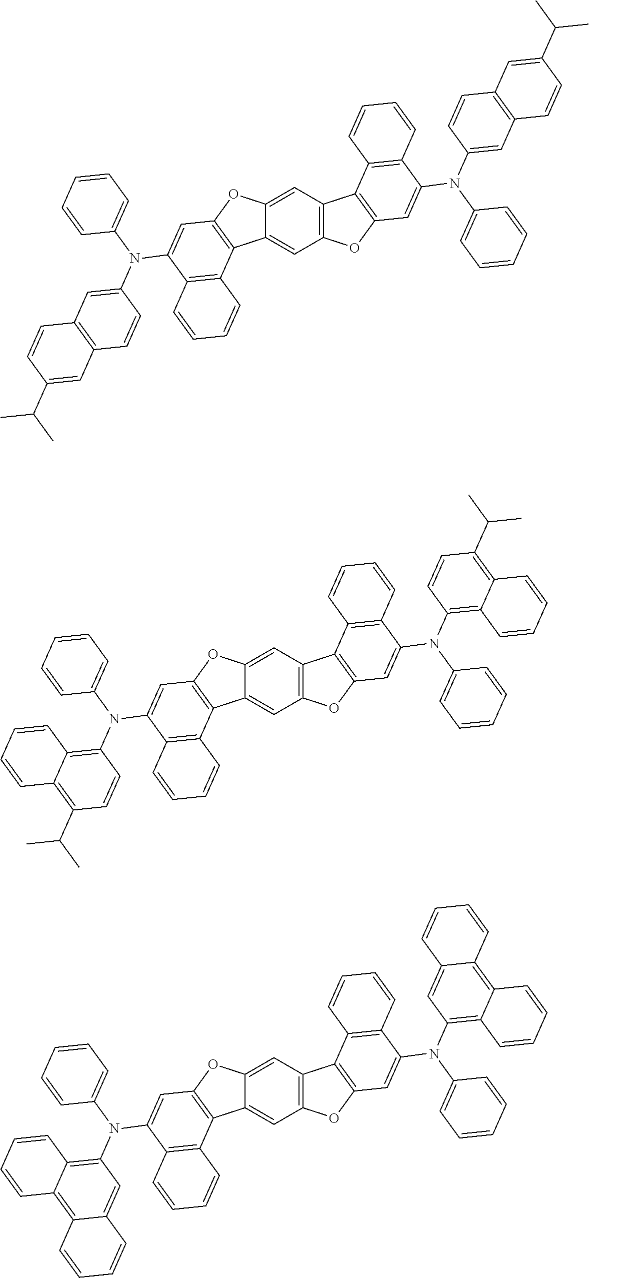 [SMILES: CC(C)c1ccc2cc(N(c3ccccc3)c3cc4oc5cc6c(cc5c4c4ccccc34)oc3cc(N(c4ccccc4)c4ccc5cc(C(C)C)ccc5c4)c4ccccc4c36)ccc2c1 ; CC(C)c1ccc(N(c2ccccc2)c2cc3oc4cc5c(cc4c3c3ccccc23)oc2cc(N(c3ccccc3)c3ccc(C(C)C)c4ccccc34)c3ccccc3c25)c2ccccc12 ; c1ccc(N(c2cc3ccccc3c3ccccc23)c2cc3oc4cc5c(cc4c3c3ccccc23)oc2cc(N(c3ccccc3)c3cc4ccccc4c4ccccc34)c3ccccc3c25)cc1]

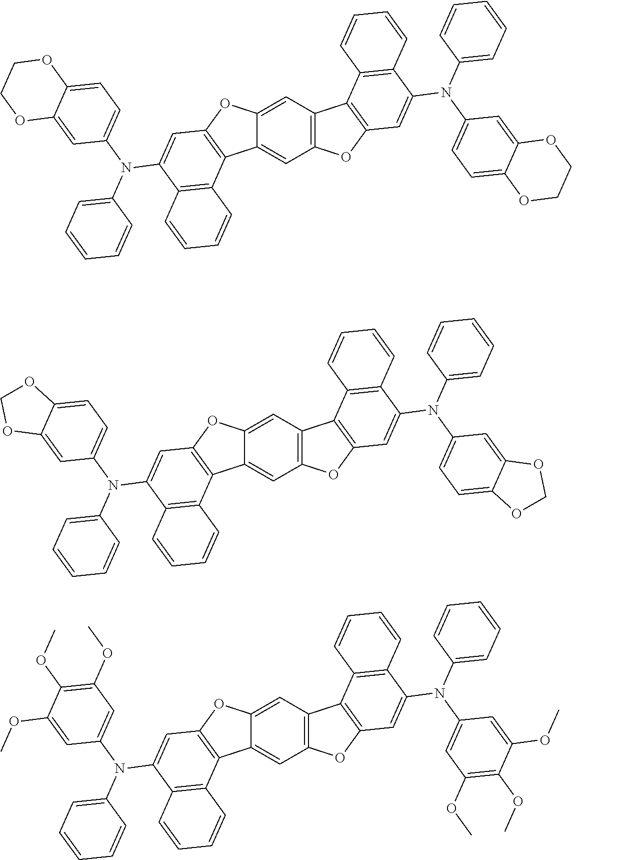

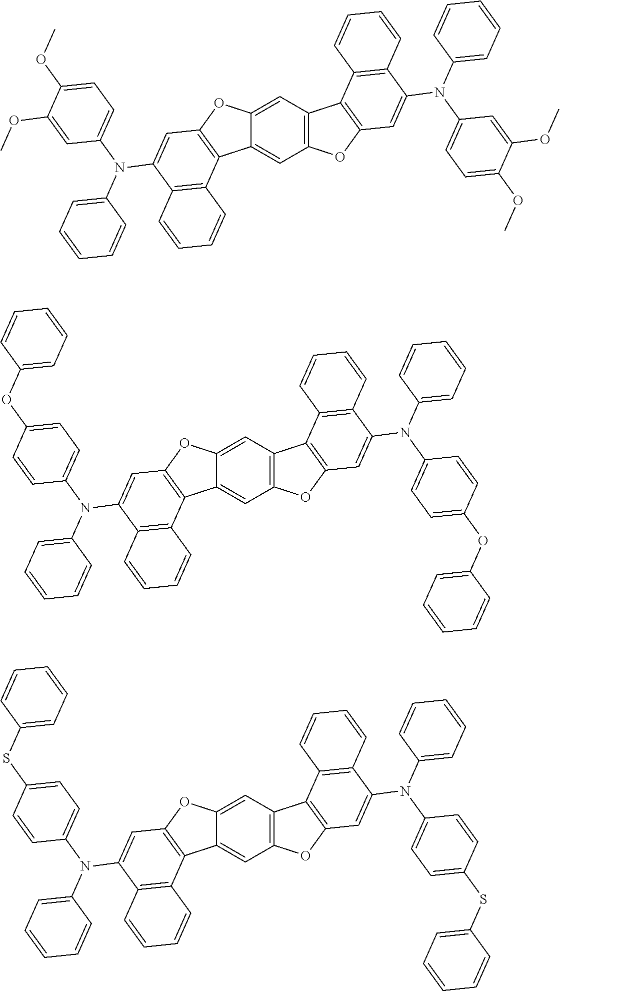

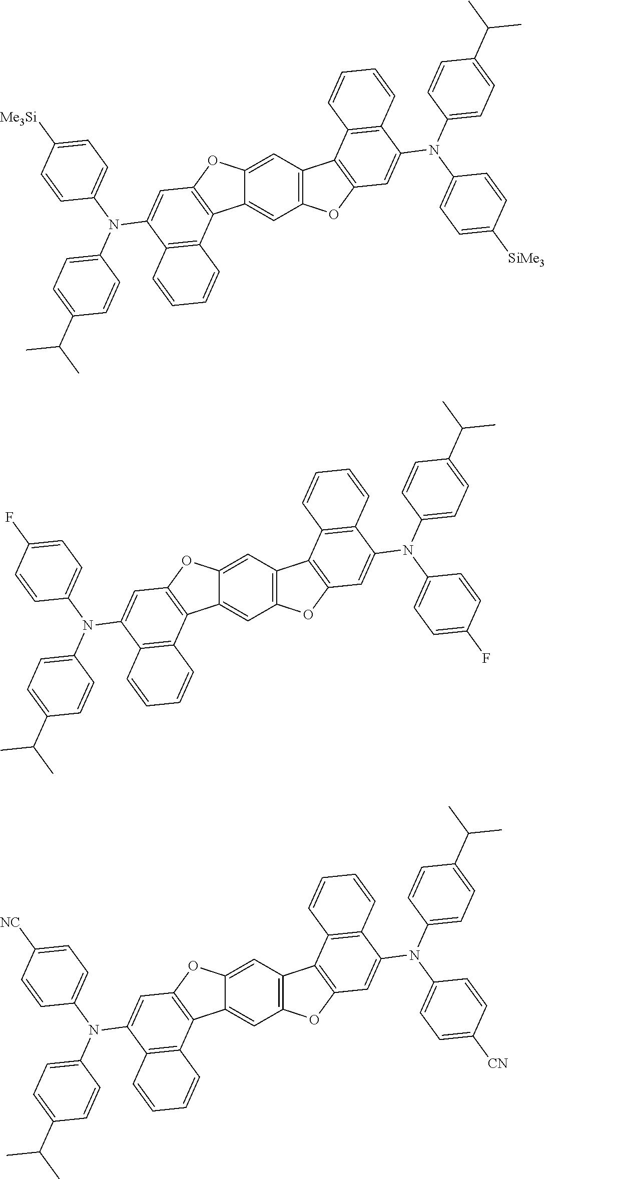

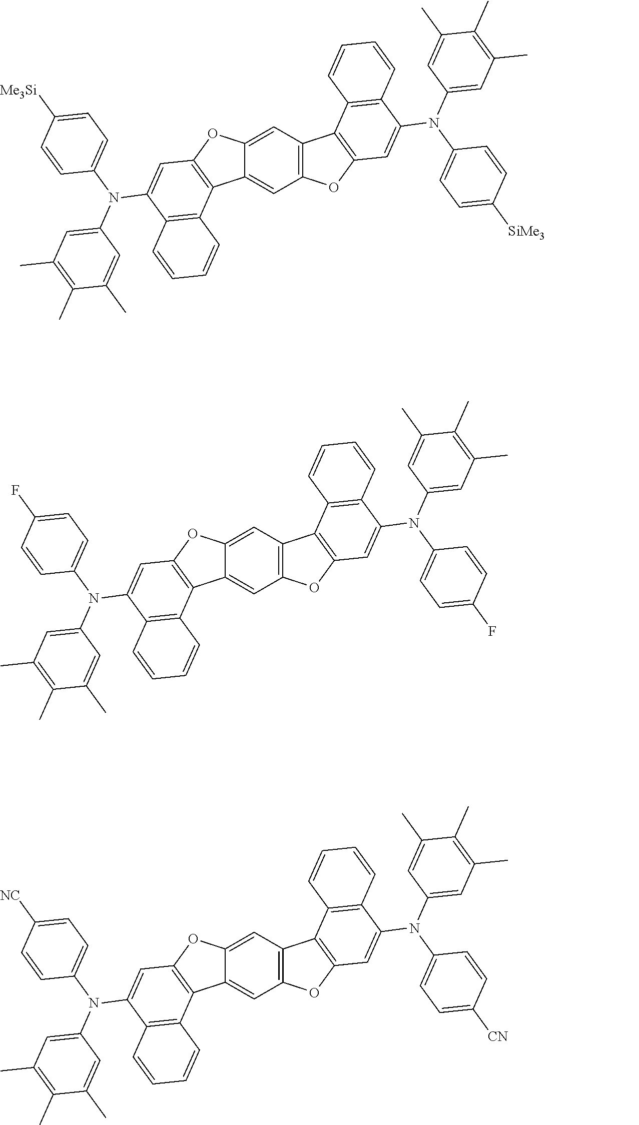

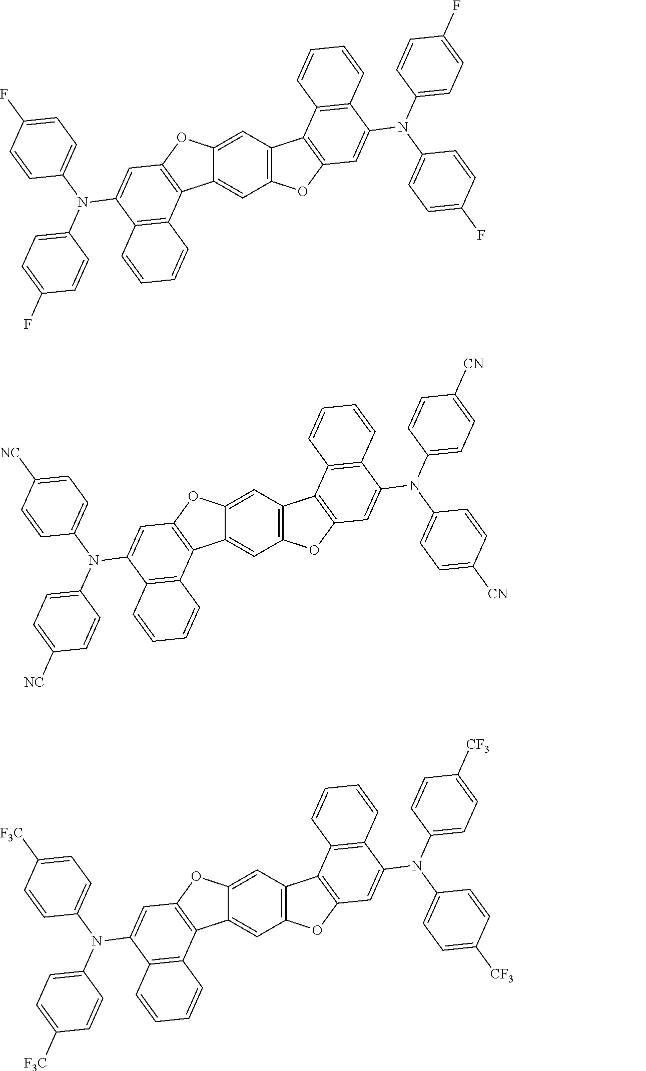 [SMILES: Fc1ccc(N(c2ccc(F)cc2)c2cc3oc4cc5c(cc4c3c3ccccc23)oc2cc(N(c3ccc(F)cc3)c3ccc(F)cc3)c3ccccc3c25)cc1 ; N#Cc1ccc(N(c2ccc(C#N)cc2)c2cc3oc4cc5c(cc4c3c3ccccc23)oc2cc(N(c3ccc(C#N)cc3)c3ccc(C#N)cc3)c3ccccc3c25)cc1 ; FC(F)(F)c1ccc(N(c2ccc(C(F)(F)F)cc2)c2cc3oc4cc5c(cc4c3c3ccccc23)oc2cc(N(c3ccc(C(F)(F)F)cc3)c3ccc(C(F)(F)F)cc3)c3ccccc3c25)cc1]

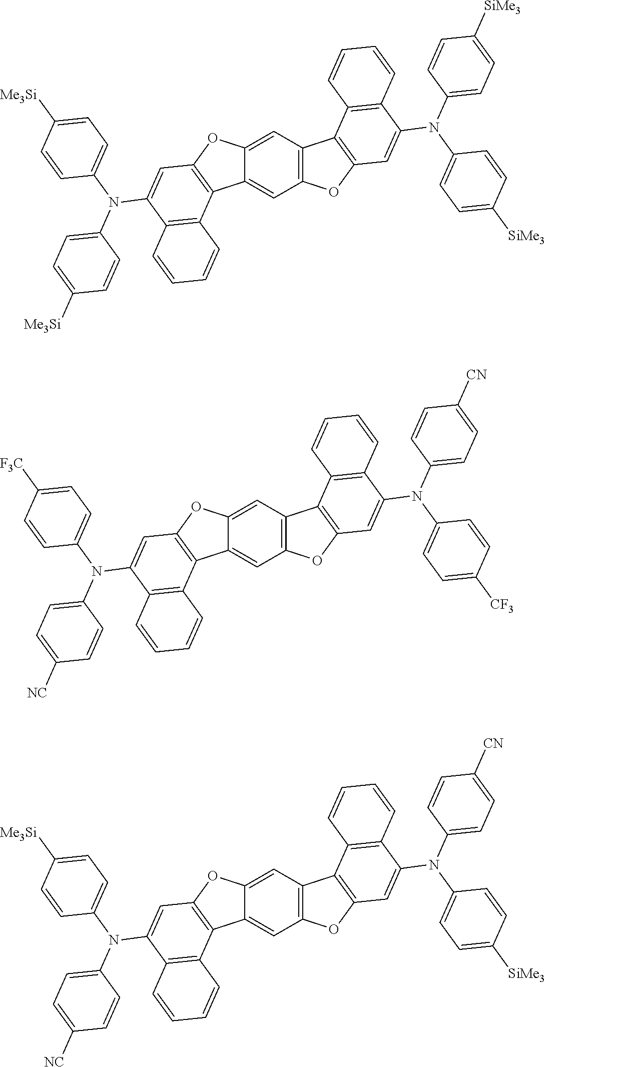 [SMILES: C[Si](C)(C)c1ccc(N(c2ccc([Si](C)(C)C)cc2)c2cc3oc4cc5c(cc4c3c3ccccc23)oc2cc(N(c3ccc([Si](C)(C)C)cc3)c3ccc([Si](C)(C)C)cc3)c3ccccc3c25)cc1 ; N#Cc1ccc(N(c2ccc(C(F)(F)F)cc2)c2cc3oc4cc5c(cc4c3c3ccccc23)oc2cc(N(c3ccc(C#N)cc3)c3ccc(C(F)(F)F)cc3)c3ccccc3c25)cc1 ; C[Si](C)(C)c1ccc(N(c2ccc(C#N)cc2)c2cc3oc4cc5c(cc4c3c3ccccc23)oc2cc(N(c3ccc(C#N)cc3)c3ccc([Si](C)(C)C)cc3)c3ccccc3c25)cc1]

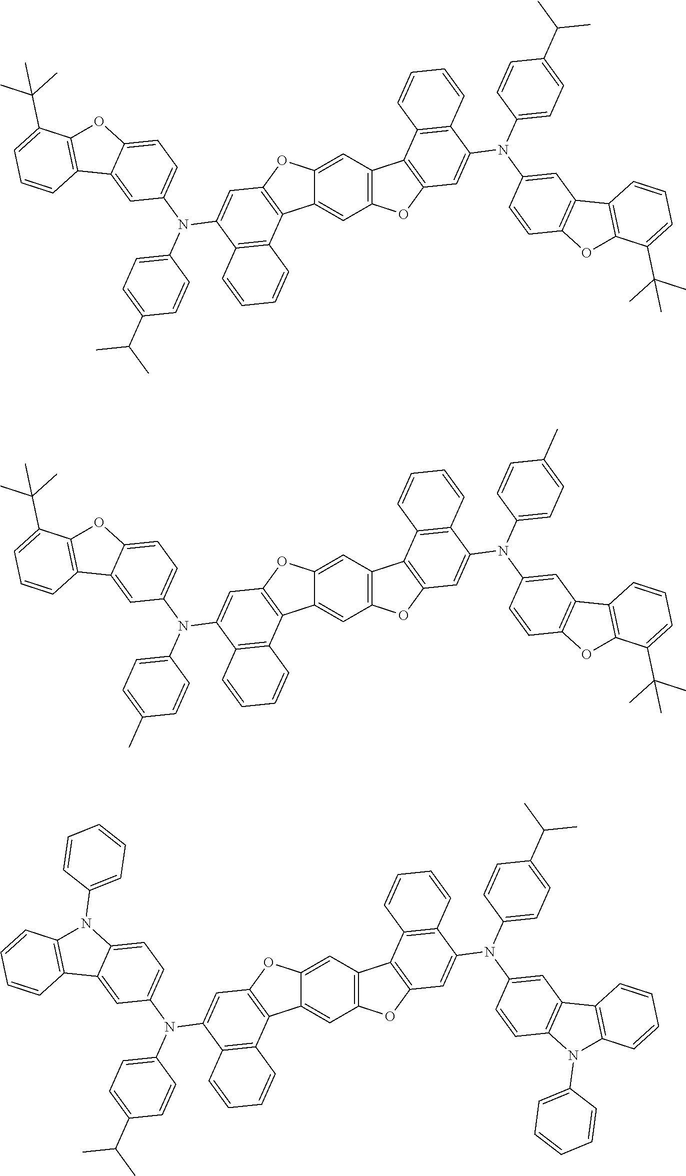

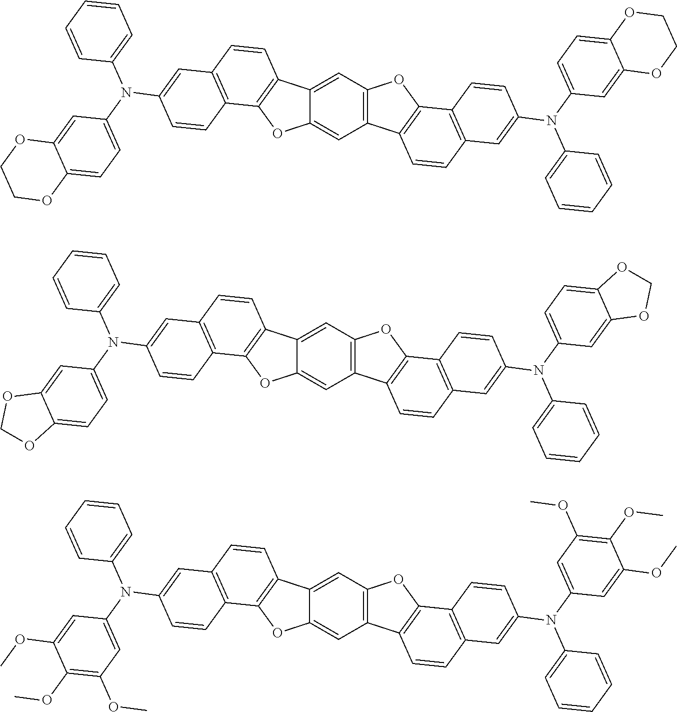

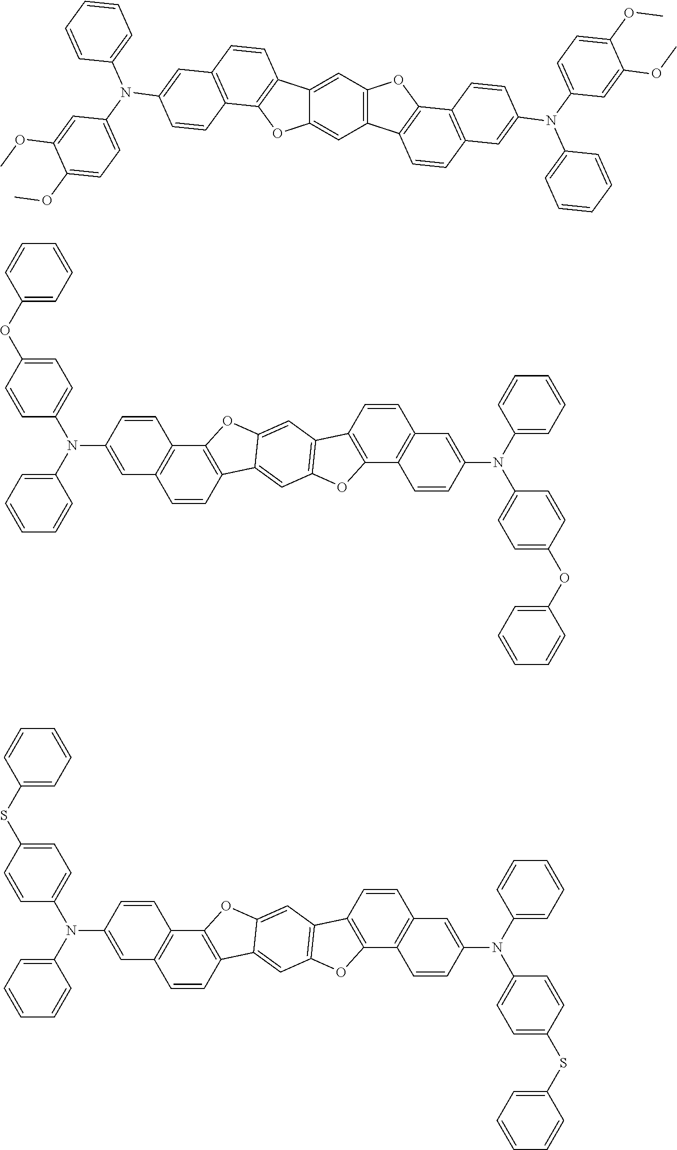

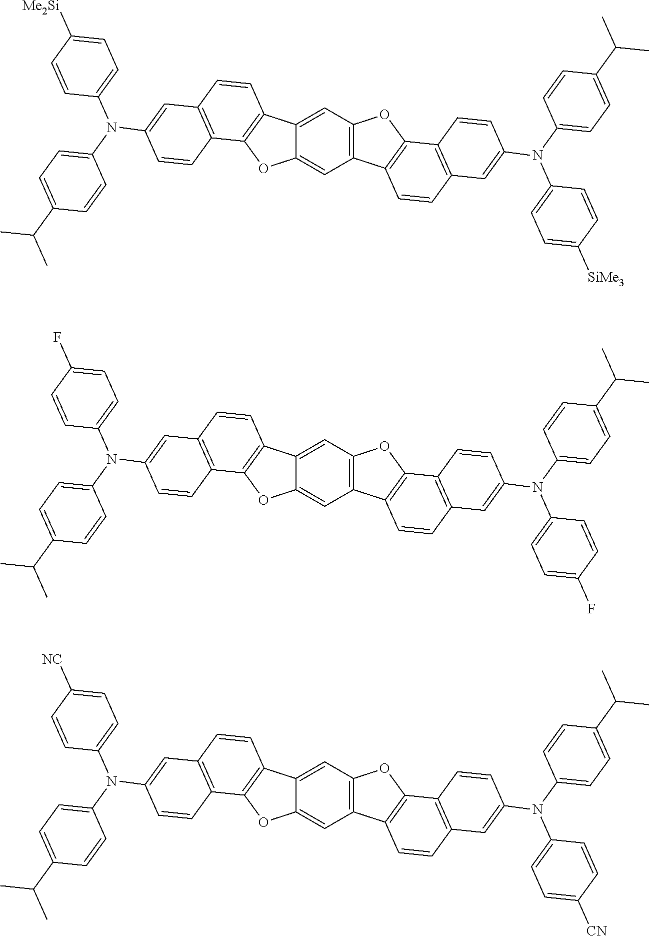

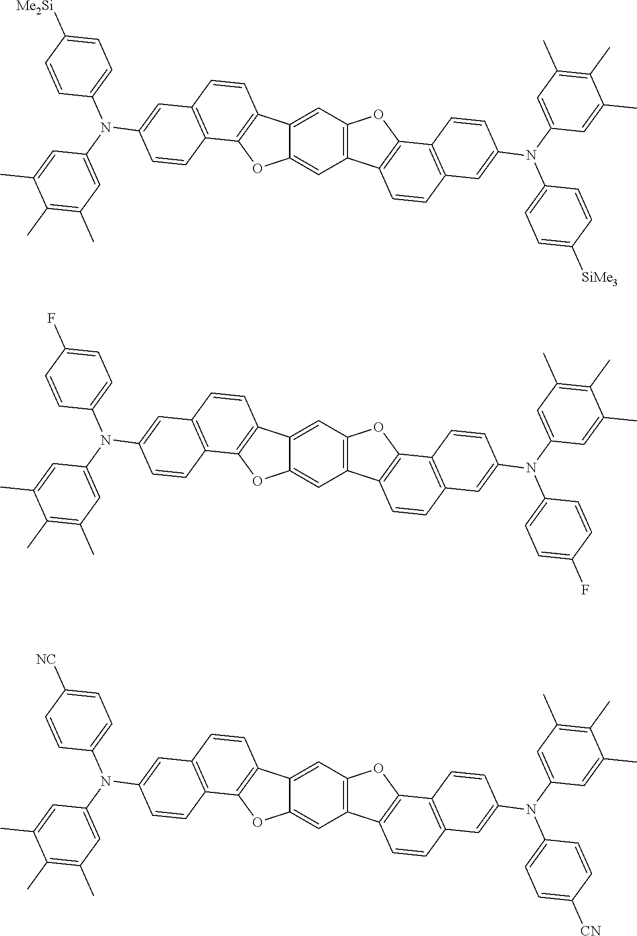

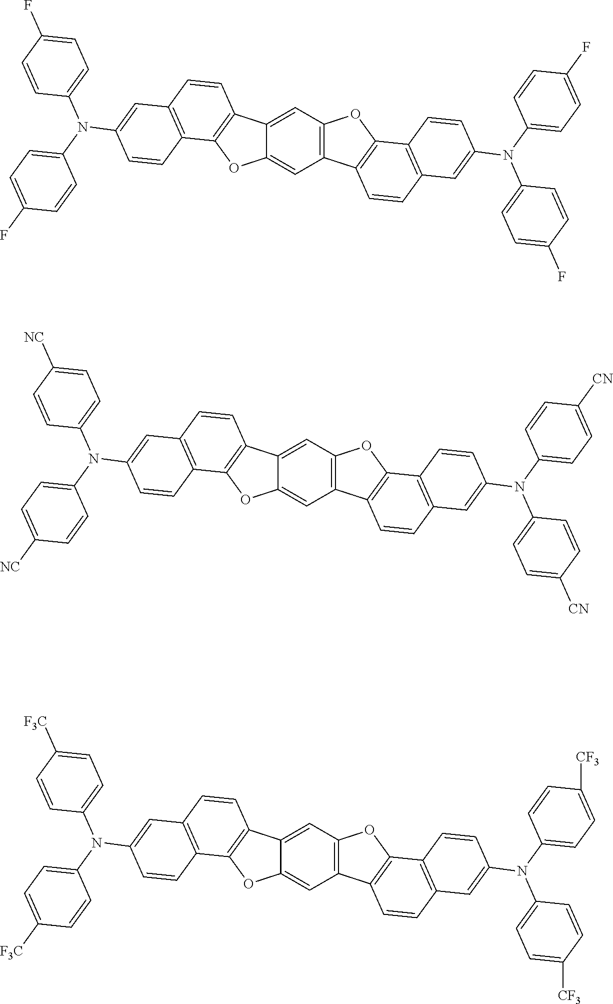 [SMILES: Fc1ccc(N(c2ccc(F)cc2)c2ccc3c(ccc4c5cc6oc7c8ccc(N(c9ccc(F)cc9)c9ccc(F)cc9)cc8ccc7c6cc5oc34)c2)cc1 ; N#Cc1ccc(N(c2ccc(C#N)cc2)c2ccc3c(ccc4c5cc6oc7c8ccc(N(c9ccc(C#N)cc9)c9ccc(C#N)cc9)cc8ccc7c6cc5oc34)c2)cc1 ; FC(F)(F)c1ccc(N(c2ccc(C(F)(F)F)cc2)c2ccc3c(ccc4c5cc6oc7c8ccc(N(c9ccc(C(F)(F)F)cc9)c9ccc(C(F)(F)F)cc9)cc8ccc7c6cc5oc34)c2)cc1]

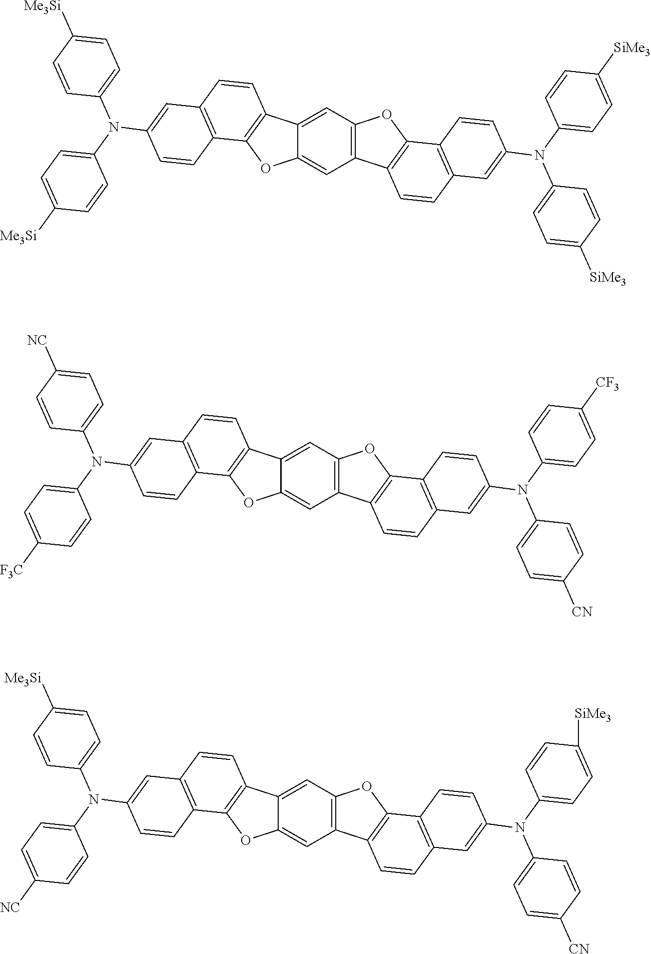 [SMILES: C[Si](C)(C)c1ccc(N(c2ccc([Si](C)(C)C)cc2)c2ccc3c(ccc4c5cc6oc7c8ccc(N(c9ccc([Si](C)(C)C)cc9)c9ccc([Si](C)(C)C)cc9)cc8ccc7c6cc5oc34)c2)cc1 ; N#Cc1ccc(N(c2ccc(C(F)(F)F)cc2)c2ccc3c(ccc4c5cc6oc7c8ccc(N(c9ccc(C#N)cc9)c9ccc(C(F)(F)F)cc9)cc8ccc7c6cc5oc34)c2)cc1 ; C[Si](C)(C)c1ccc(N(c2ccc(C#N)cc2)c2ccc3c(ccc4c5cc6oc7c8ccc(N(c9ccc(C#N)cc9)c9ccc([Si](C)(C)C)cc9)cc8ccc7c6cc5oc34)c2)cc1]

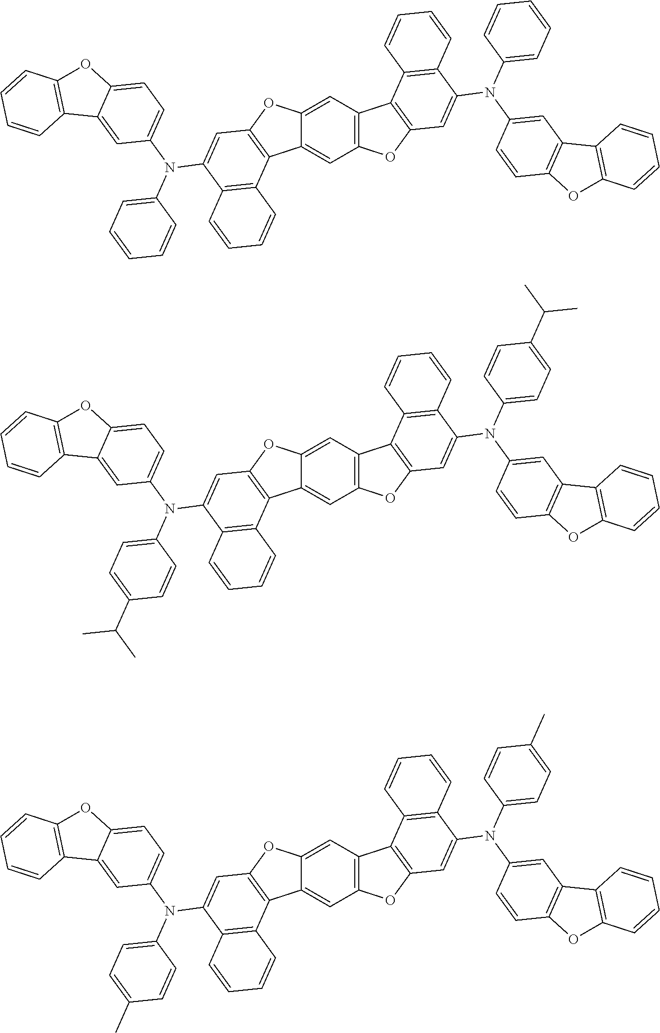 [SMILES: c1ccc(N(c2ccc3oc4ccccc4c3c2)c2cc3oc4cc5c(cc4c3c3ccccc23)oc2cc(N(c3ccccc3)c3ccc4oc6ccccc6c4c3)c3ccccc3c25)cc1 ; CC(C)c1ccc(N(c2ccc3oc4ccccc4c3c2)c2cc3oc4cc5c(cc4c3c3ccccc23)oc2cc(N(c3ccc(C(C)C)cc3)c3ccc4oc6ccccc6c4c3)c3ccccc3c25)cc1 ; Cc1ccc(N(c2ccc3oc4ccccc4c3c2)c2cc3oc4cc5c(cc4c3c3ccccc23)oc2cc(N(c3ccc(C)cc3)c3ccc4oc6ccccc6c4c3)c3ccccc3c25)cc1]

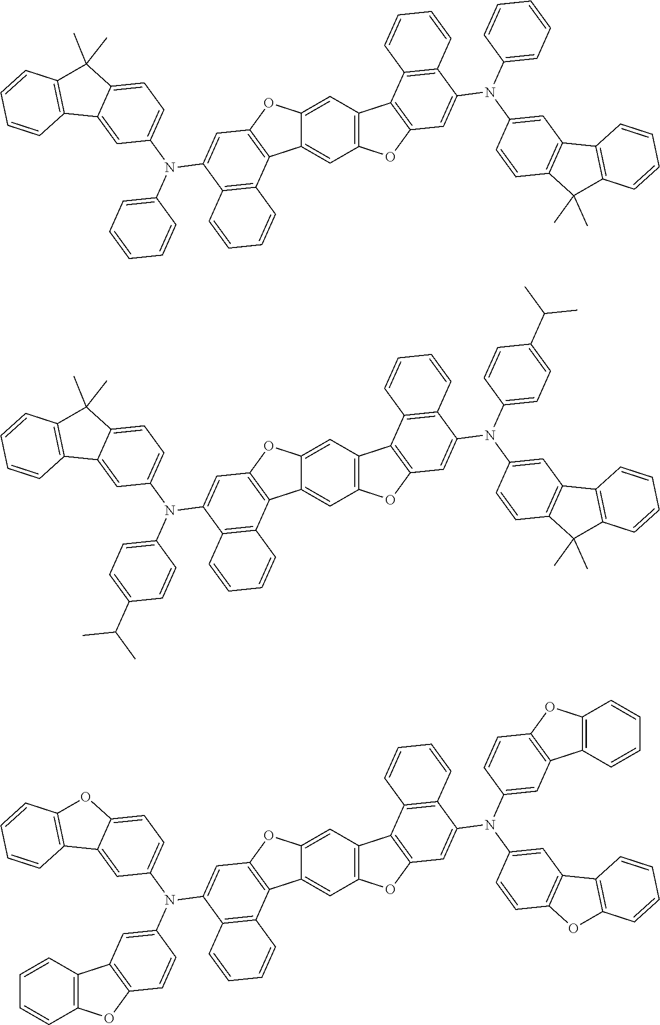 [SMILES: CC1(C)c2ccccc2-c2cc(N(c3ccccc3)c3cc4oc5cc6c(cc5c4c4ccccc34)oc3cc(N(c4ccccc4)c4ccc5c(c4)-c4ccccc4C5(C)C)c4ccccc4c36)ccc21 ; CC(C)c1ccc(N(c2ccc3c(c2)-c2ccccc2C3(C)C)c2cc3oc4cc5c(cc4c3c3ccccc23)oc2cc(N(c3ccc(C(C)C)cc3)c3ccc4c(c3)-c3ccccc3C4(C)C)c3ccccc3c25)cc1 ; c1ccc2c(c1)oc1ccc(N(c3ccc4oc5ccccc5c4c3)c3cc4oc5cc6c(cc5c4c4ccccc34)oc3cc(N(c4ccc5oc7ccccc7c5c4)c4ccc5oc7ccccc7c5c4)c4ccccc4c36)cc12]

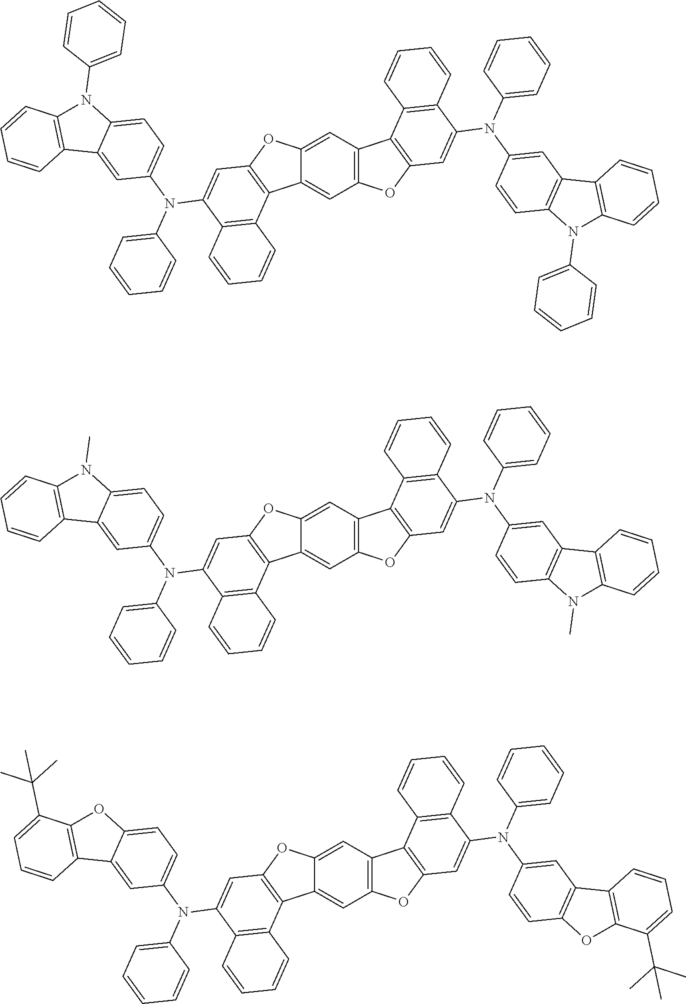 [SMILES: c1ccc(N(c2ccc3c(c2)c2ccccc2n3-c2ccccc2)c2cc3oc4cc5c(cc4c3c3ccccc23)oc2cc(N(c3ccccc3)c3ccc4c(c3)c3ccccc3n4-c3ccccc3)c3ccccc3c25)cc1 ; Cn1c2ccccc2c2cc(N(c3ccccc3)c3cc4oc5cc6c(cc5c4c4ccccc34)oc3cc(N(c4ccccc4)c4ccc5c(c4)c4ccccc4n5C)c4ccccc4c36)ccc21 ; CC(C)(C)c1cccc2c1oc1ccc(N(c3ccccc3)c3cc4oc5cc6c(cc5c4c4ccccc34)oc3cc(N(c4ccccc4)c4ccc5oc7c(C(C)(C)C)cccc7c5c4)c4ccccc4c36)cc12]

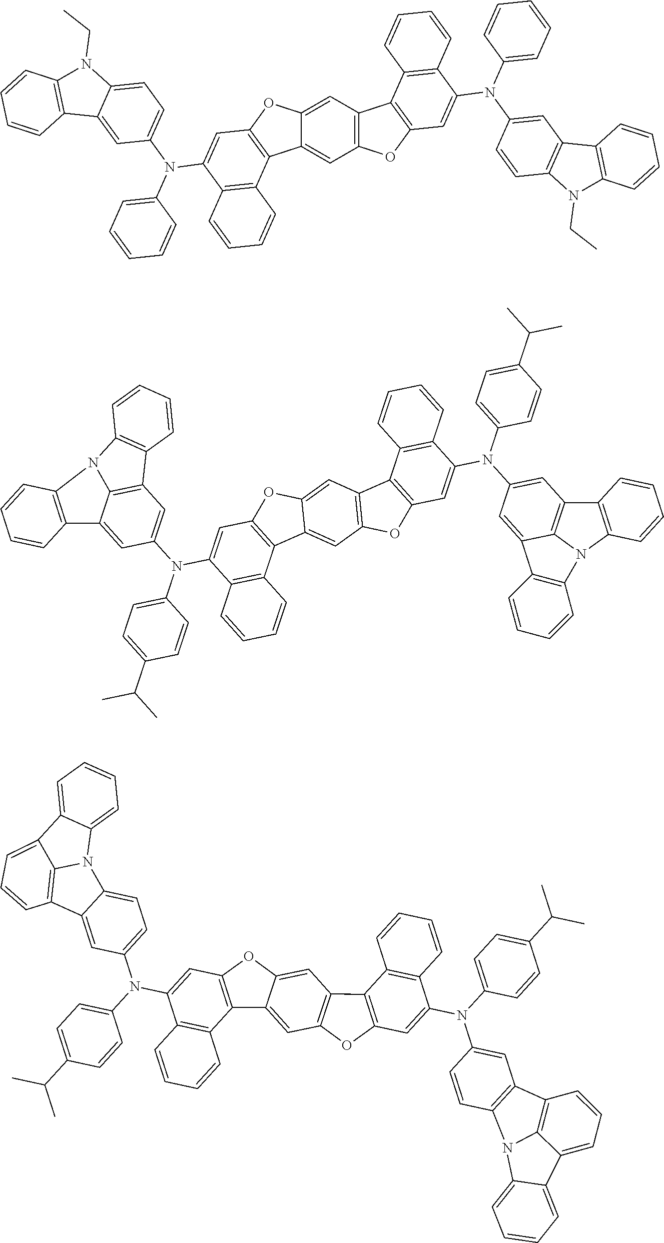 [SMILES: CCn1c2ccccc2c2cc(N(c3ccccc3)c3cc4oc5cc6c(cc5c4c4ccccc34)oc3cc(N(c4ccccc4)c4ccc5c(c4)c4ccccc4n5CC)c4ccccc4c36)ccc21 ; CC(C)c1ccc(N(c2cc3c4ccccc4n4c5ccccc5c(c2)c34)c2cc3oc4cc5c(cc4c3c3ccccc23)oc2cc(N(c3ccc(C(C)C)cc3)c3cc4c6ccccc6n6c7ccccc7c(c3)c46)c3ccccc3c25)cc1 ; CC(C)c1ccc(N(c2ccc3c(c2)c2cccc4c5ccccc5n3c42)c2cc3oc4cc5c(cc4c3c3ccccc23)oc2cc(N(c3ccc(C(C)C)cc3)c3ccc4c(c3)c3cccc6c7ccccc7n4c63)c3ccccc3c25)cc1]

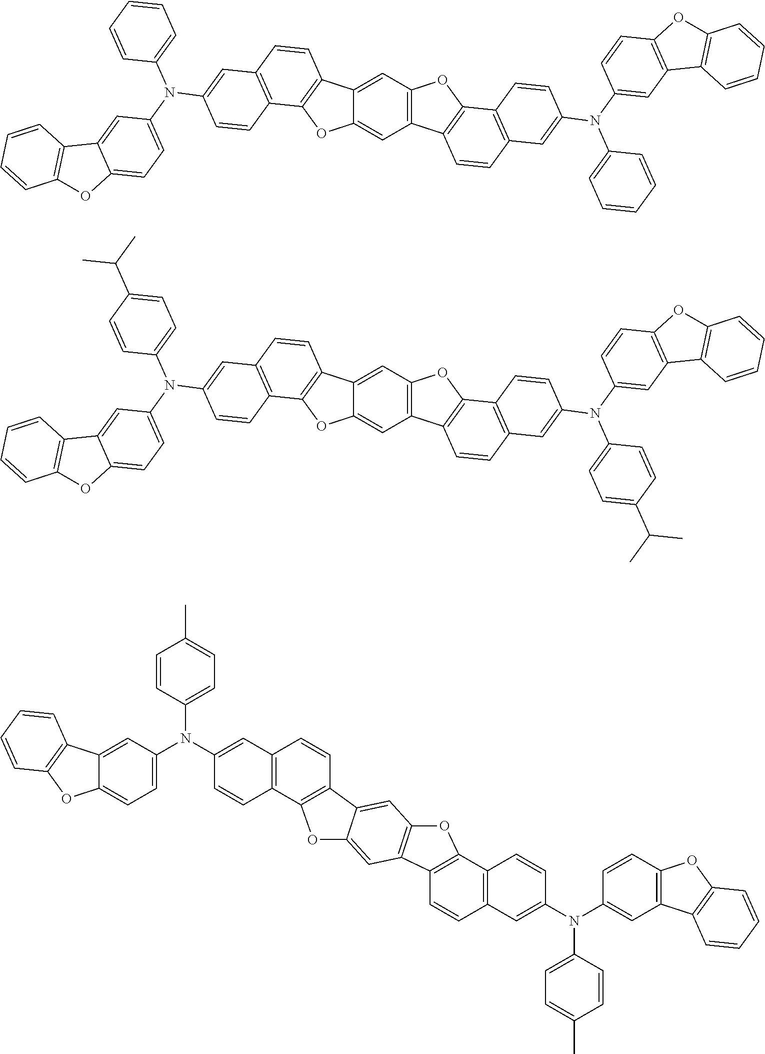 [SMILES: c1ccc(N(c2ccc3c(ccc4c5cc6oc7c8ccc(N(c9ccccc9)c9ccc%10oc%11ccccc%11c%10c9)cc8ccc7c6cc5oc34)c2)c2ccc3oc4ccccc4c3c2)cc1 ; CC(C)c1ccc(N(c2ccc3c(ccc4c5cc6oc7c8ccc(N(c9ccc(C(C)C)cc9)c9ccc%10oc%11ccccc%11c%10c9)cc8ccc7c6cc5oc34)c2)c2ccc3oc4ccccc4c3c2)cc1 ; Cc1ccc(N(c2ccc3c(ccc4c5cc6oc7c8ccc(N(c9ccc(C)cc9)c9ccc%10oc%11ccccc%11c%10c9)cc8ccc7c6cc5oc34)c2)c2ccc3oc4ccccc4c3c2)cc1]

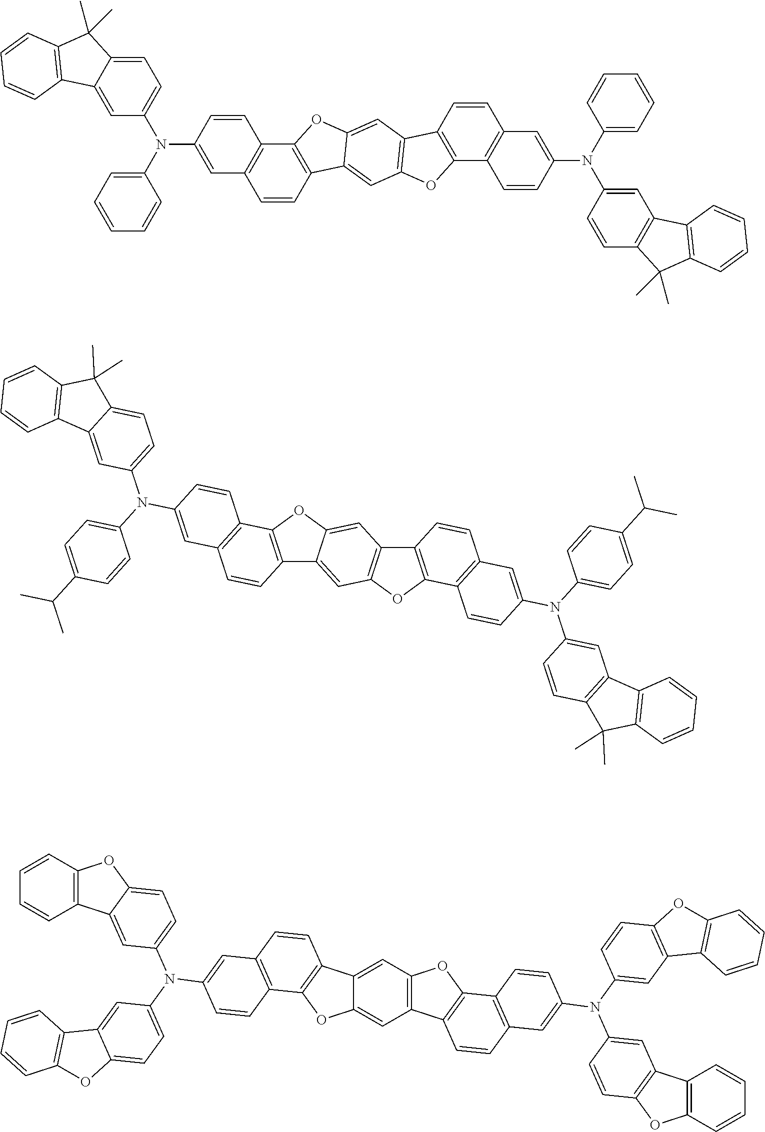 [SMILES: CC1(C)c2ccccc2-c2cc(N(c3ccccc3)c3ccc4c(ccc5c6cc7oc8c9ccc(N(c%10ccccc%10)c%10ccc%11c(c%10)-c%10ccccc%10C%11(C)C)cc9ccc8c7cc6oc45)c3)ccc21 ; CC(C)c1ccc(N(c2ccc3c(c2)-c2ccccc2C3(C)C)c2ccc3c(ccc4c5cc6oc7c8ccc(N(c9ccc(C(C)C)cc9)c9ccc%10c(c9)-c9ccccc9C%10(C)C)cc8ccc7c6cc5oc34)c2)cc1 ; c1ccc2c(c1)oc1ccc(N(c3ccc4c(ccc5c6cc7oc8c9ccc(N(c%10ccc%11oc%12ccccc%12c%11c%10)c%10ccc%11oc%12ccccc%12c%11c%10)cc9ccc8c7cc6oc45)c3)c3ccc4oc5ccccc5c4c3)cc12]

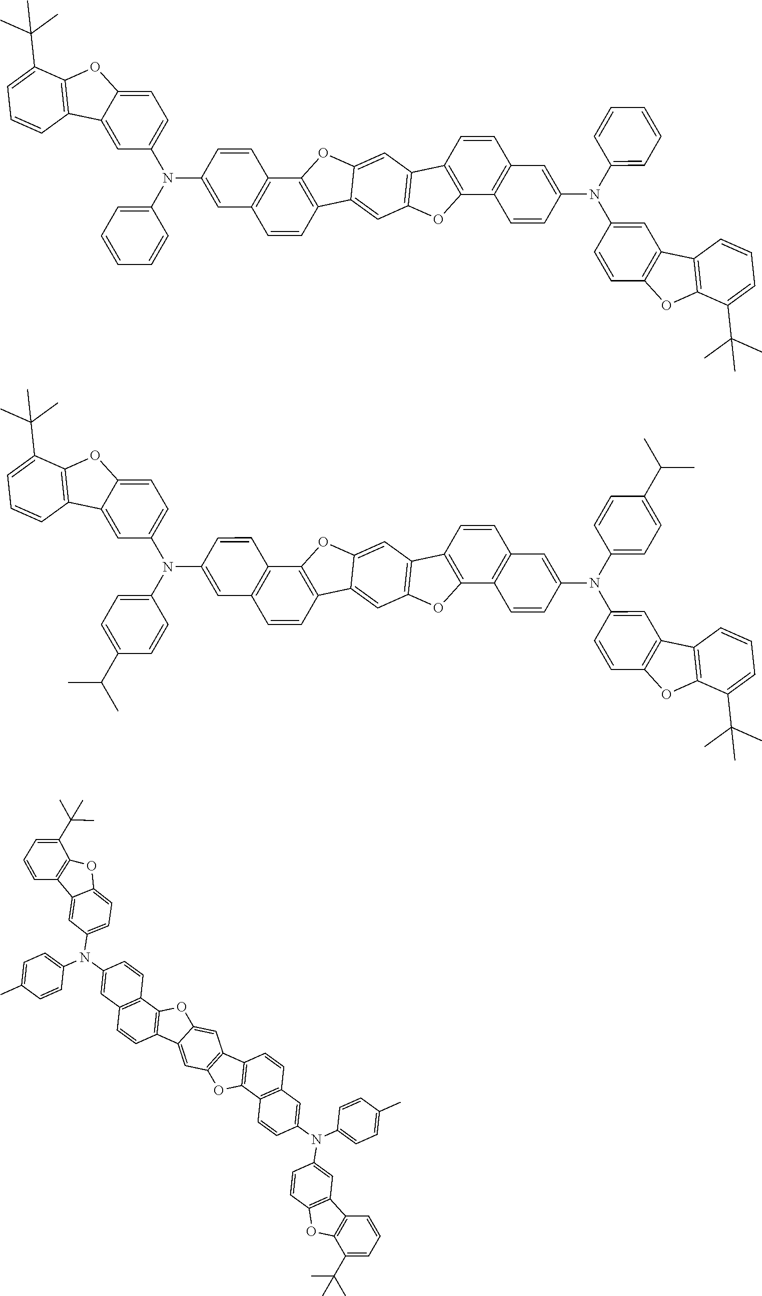

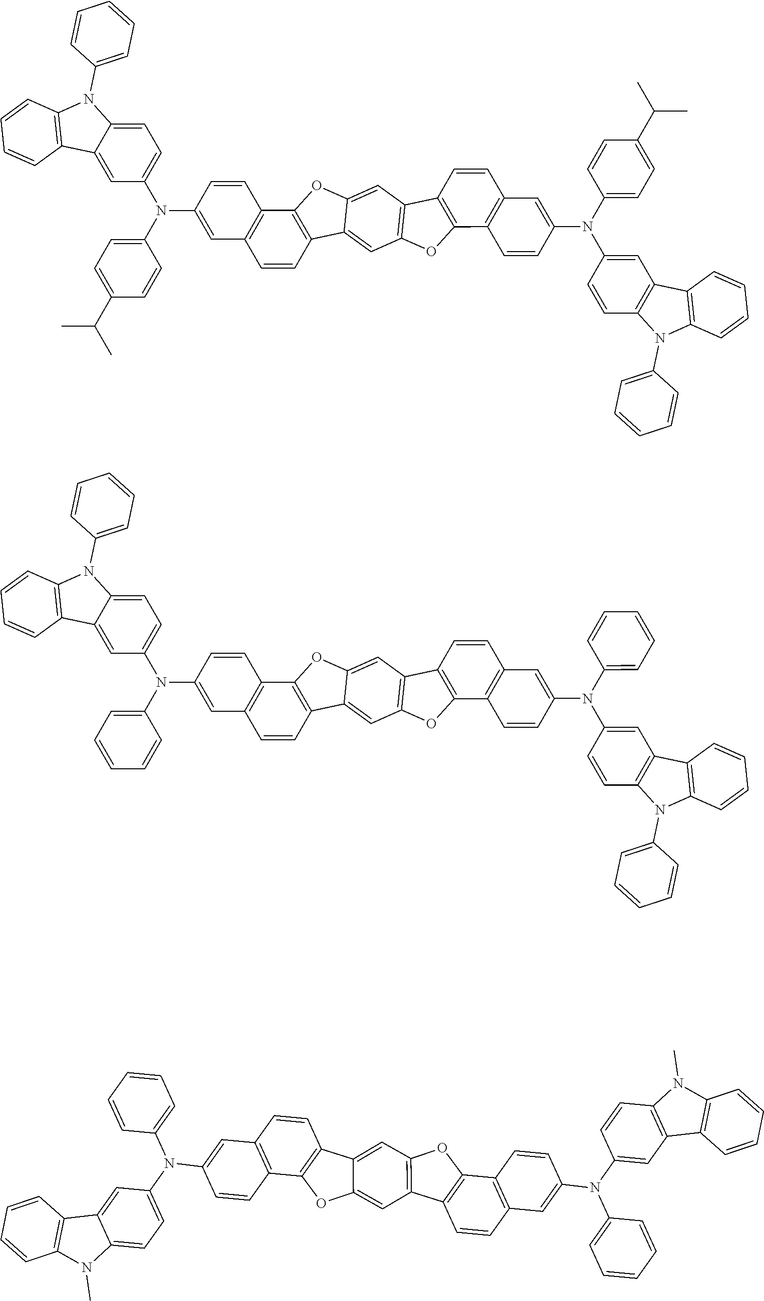 [SMILES: CC(C)c1ccc(N(c2ccc3c(ccc4c5cc6oc7c8ccc(N(c9ccc(C(C)C)cc9)c9ccc%10c(c9)c9ccccc9n%10-c9ccccc9)cc8ccc7c6cc5oc34)c2)c2ccc3c(c2)c2ccccc2n3-c2ccccc2)cc1 ; c1ccc(N(c2ccc3c(ccc4c5cc6oc7c8ccc(N(c9ccccc9)c9ccc%10c(c9)c9ccccc9n%10-c9ccccc9)cc8ccc7c6cc5oc34)c2)c2ccc3c(c2)c2ccccc2n3-c2ccccc2)cc1 ; Cn1c2ccccc2c2cc(N(c3ccccc3)c3ccc4c(ccc5c6cc7oc8c9ccc(N(c%10ccccc%10)c%10ccc%11c(c%10)c%10ccccc%10n%11C)cc9ccc8c7cc6oc45)c3)ccc21]

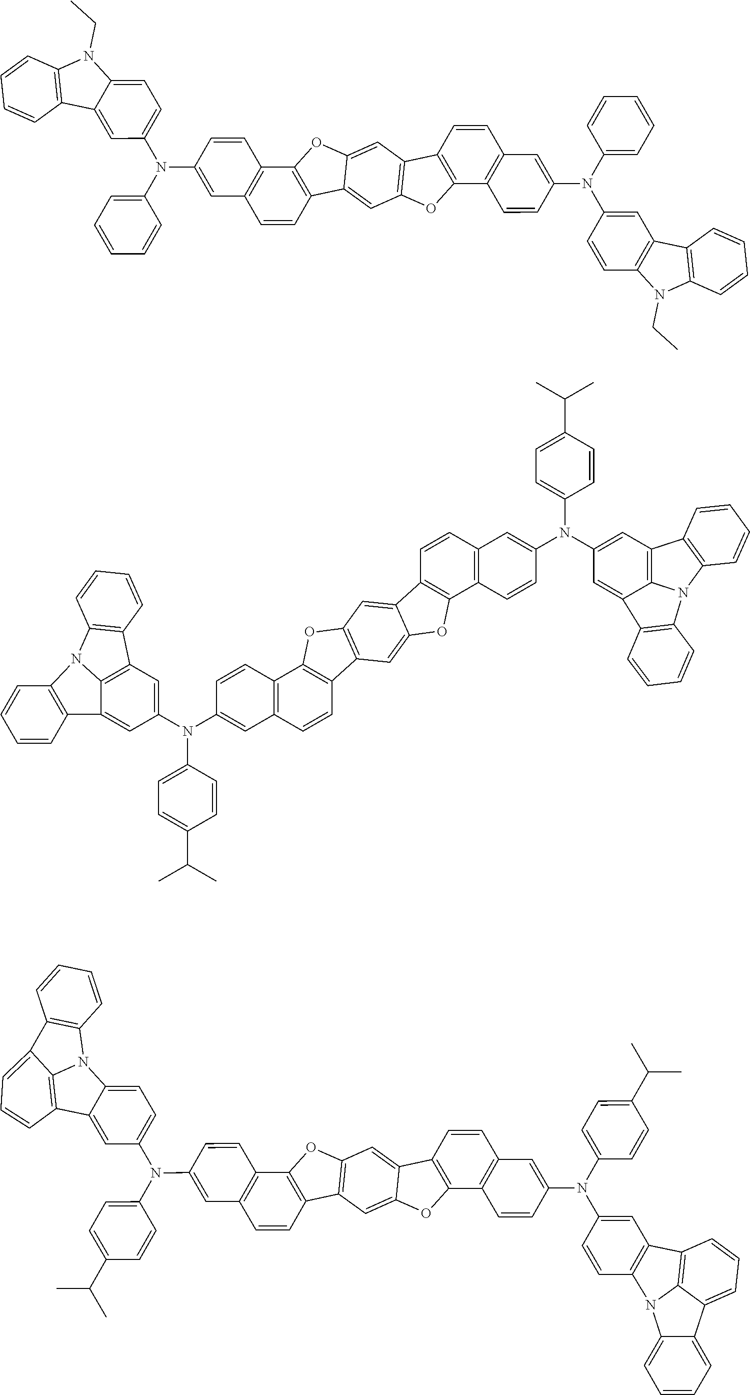 [SMILES: CCn1c2ccccc2c2cc(N(c3ccccc3)c3ccc4c(ccc5c6cc7oc8c9ccc(N(c%10ccccc%10)c%10ccc%11c(c%10)c%10ccccc%10n%11CC)cc9ccc8c7cc6oc45)c3)ccc21 ; CC(C)c1ccc(N(c2ccc3c(ccc4c5cc6oc7c8ccc(N(c9ccc(C(C)C)cc9)c9cc%10c%11ccccc%11n%11c%12ccccc%12c(c9)c%10%11)cc8ccc7c6cc5oc34)c2)c2cc3c4ccccc4n4c5ccccc5c(c2)c34)cc1 ; CC(C)c1ccc(N(c2ccc3c(ccc4c5cc6oc7c8ccc(N(c9ccc(C(C)C)cc9)c9ccc%10c(c9)c9cccc%11c%12ccccc%12n%10c%119)cc8ccc7c6cc5oc34)c2)c2ccc3c(c2)c2cccc4c5ccccc5n3c42)cc1]

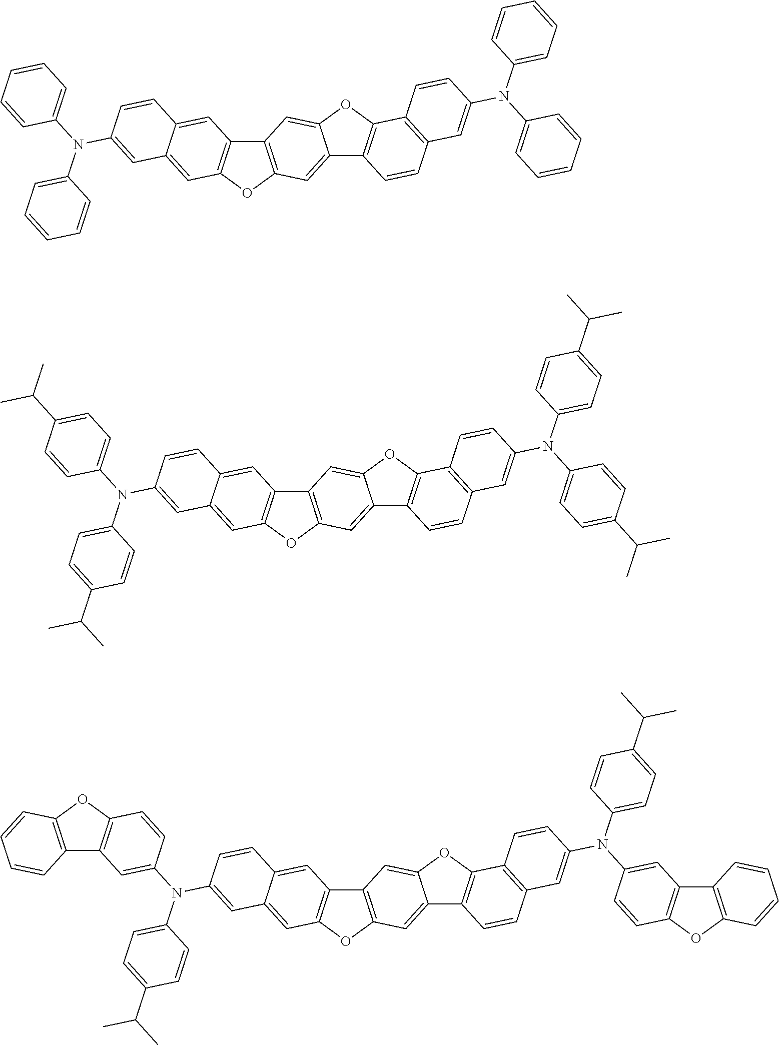 [SMILES: c1ccc(N(c2ccccc2)c2ccc3cc4c(cc3c2)oc2cc3c(cc24)oc2c4ccc(N(c5ccccc5)c5ccccc5)cc4ccc32)cc1 ; CC(C)c1ccc(N(c2ccc(C(C)C)cc2)c2ccc3cc4c(cc3c2)oc2cc3c(cc24)oc2c4ccc(N(c5ccc(C(C)C)cc5)c5ccc(C(C)C)cc5)cc4ccc32)cc1 ; CC(C)c1ccc(N(c2ccc3cc4c(cc3c2)oc2cc3c(cc24)oc2c4ccc(N(c5ccc(C(C)C)cc5)c5ccc6oc7ccccc7c6c5)cc4ccc32)c2ccc3oc4ccccc4c3c2)cc1]

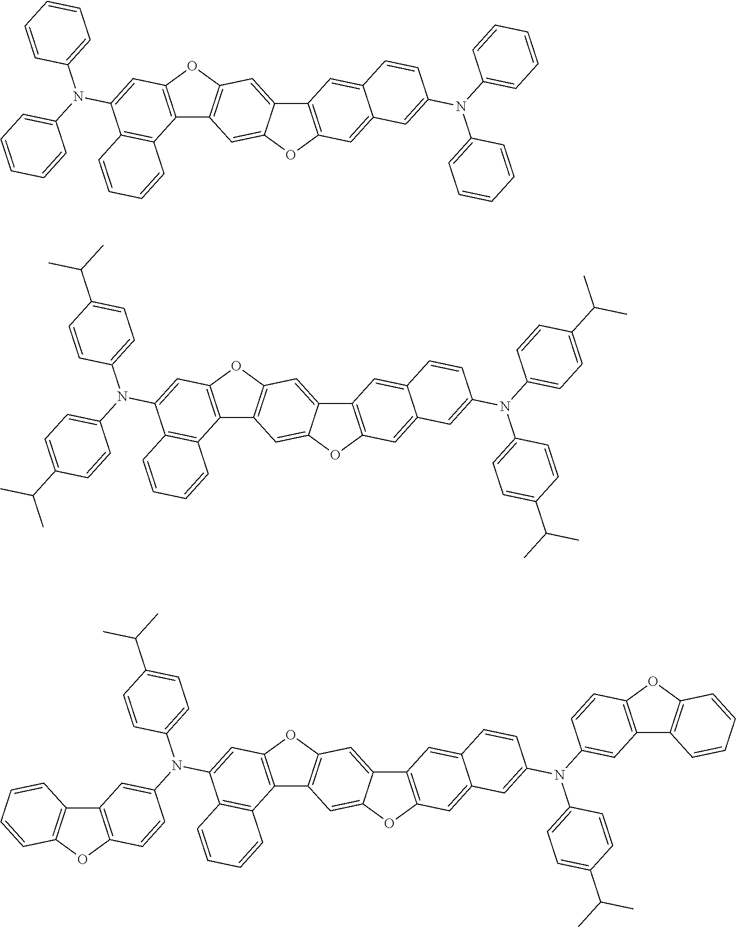 [SMILES: c1ccc(N(c2ccccc2)c2ccc3cc4c(cc3c2)oc2cc3c(cc24)oc2cc(N(c4ccccc4)c4ccccc4)c4ccccc4c23)cc1 ; CC(C)c1ccc(N(c2ccc(C(C)C)cc2)c2ccc3cc4c(cc3c2)oc2cc3c(cc24)oc2cc(N(c4ccc(C(C)C)cc4)c4ccc(C(C)C)cc4)c4ccccc4c23)cc1 ; CC(C)c1ccc(N(c2ccc3cc4c(cc3c2)oc2cc3c(cc24)oc2cc(N(c4ccc(C(C)C)cc4)c4ccc5oc6ccccc6c5c4)c4ccccc4c23)c2ccc3oc4ccccc4c3c2)cc1]

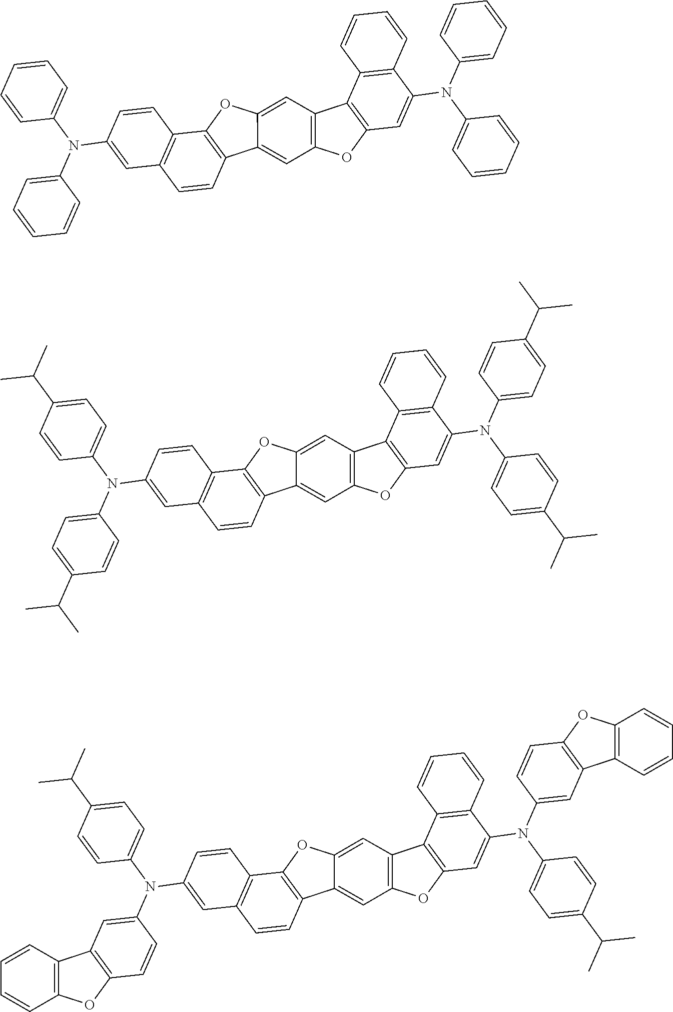 [SMILES: c1ccc(N(c2ccccc2)c2ccc3c(ccc4c5cc6oc7cc(N(c8ccccc8)c8ccccc8)c8ccccc8c7c6cc5oc34)c2)cc1 ; CC(C)c1ccc(N(c2ccc(C(C)C)cc2)c2ccc3c(ccc4c5cc6oc7cc(N(c8ccc(C(C)C)cc8)c8ccc(C(C)C)cc8)c8ccccc8c7c6cc5oc34)c2)cc1 ; CC(C)c1ccc(N(c2ccc3c(ccc4c5cc6oc7cc(N(c8ccc(C(C)C)cc8)c8ccc9oc%10ccccc%10c9c8)c8ccccc8c7c6cc5oc34)c2)c2ccc3oc4ccccc4c3c2)cc1]

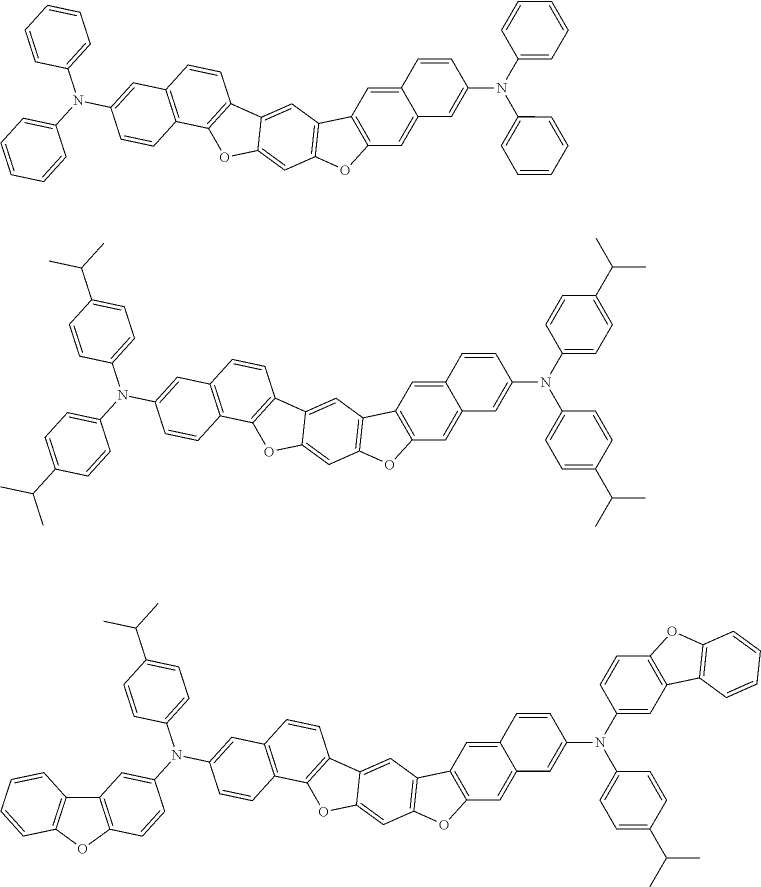 [SMILES: c1ccc(N(c2ccccc2)c2ccc3cc4c(cc3c2)oc2cc3oc5c6ccc(N(c7ccccc7)c7ccccc7)cc6ccc5c3cc24)cc1 ; CC(C)c1ccc(N(c2ccc(C(C)C)cc2)c2ccc3cc4c(cc3c2)oc2cc3oc5c6ccc(N(c7ccc(C(C)C)cc7)c7ccc(C(C)C)cc7)cc6ccc5c3cc24)cc1 ; CC(C)c1ccc(N(c2ccc3cc4c(cc3c2)oc2cc3oc5c6ccc(N(c7ccc(C(C)C)cc7)c7ccc8oc9ccccc9c8c7)cc6ccc5c3cc24)c2ccc3oc4ccccc4c3c2)cc1]

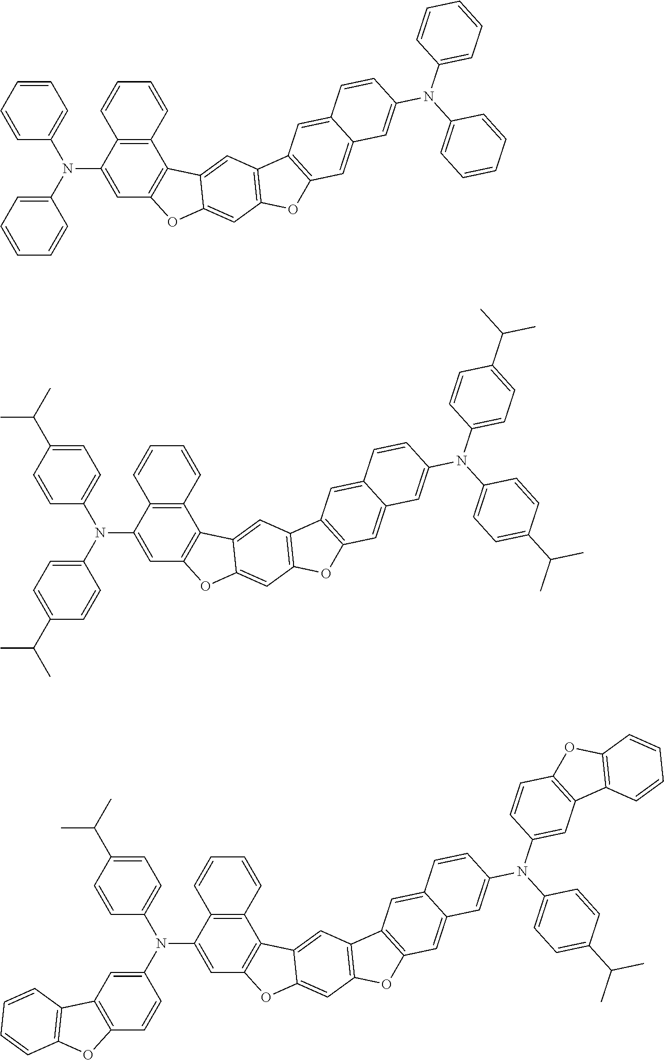 [SMILES: c1ccc(N(c2ccccc2)c2ccc3cc4c(cc3c2)oc2cc3oc5cc(N(c6ccccc6)c6ccccc6)c6ccccc6c5c3cc24)cc1 ; CC(C)c1ccc(N(c2ccc(C(C)C)cc2)c2ccc3cc4c(cc3c2)oc2cc3oc5cc(N(c6ccc(C(C)C)cc6)c6ccc(C(C)C)cc6)c6ccccc6c5c3cc24)cc1 ; CC(C)c1ccc(N(c2ccc3cc4c(cc3c2)oc2cc3oc5cc(N(c6ccc(C(C)C)cc6)c6ccc7oc8ccccc8c7c6)c6ccccc6c5c3cc24)c2ccc3oc4ccccc4c3c2)cc1]

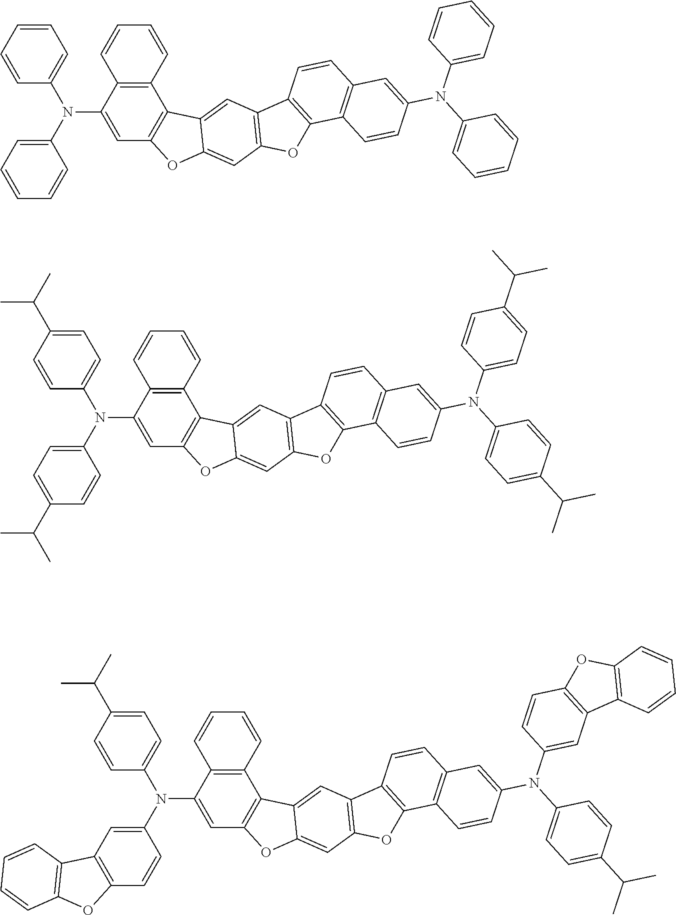 [SMILES: c1ccc(N(c2ccccc2)c2ccc3c(ccc4c5cc6c(cc5oc34)oc3cc(N(c4ccccc4)c4ccccc4)c4ccccc4c36)c2)cc1 ; CC(C)c1ccc(N(c2ccc(C(C)C)cc2)c2ccc3c(ccc4c5cc6c(cc5oc34)oc3cc(N(c4ccc(C(C)C)cc4)c4ccc(C(C)C)cc4)c4ccccc4c36)c2)cc1 ; CC(C)c1ccc(N(c2ccc3c(ccc4c5cc6c(cc5oc34)oc3cc(N(c4ccc(C(C)C)cc4)c4ccc5oc7ccccc7c5c4)c4ccccc4c36)c2)c2ccc3oc4ccccc4c3c2)cc1]

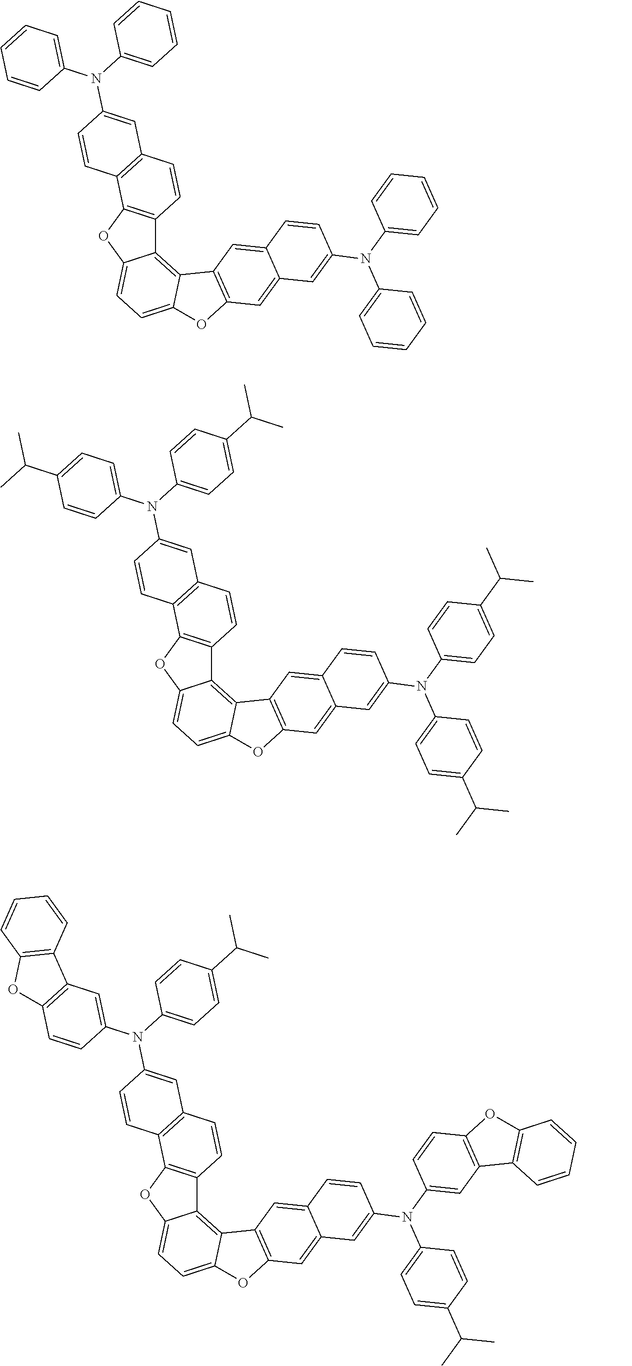 [SMILES: c1ccc(N(c2ccccc2)c2ccc3cc4c(cc3c2)oc2ccc3oc5c6ccc(N(c7ccccc7)c7ccccc7)cc6ccc5c3c24)cc1 ; CC(C)c1ccc(N(c2ccc(C(C)C)cc2)c2ccc3cc4c(cc3c2)oc2ccc3oc5c6ccc(N(c7ccc(C(C)C)cc7)c7ccc(C(C)C)cc7)cc6ccc5c3c24)cc1 ; CC(C)c1ccc(N(c2ccc3cc4c(cc3c2)oc2ccc3oc5c6ccc(N(c7ccc(C(C)C)cc7)c7ccc8oc9ccccc9c8c7)cc6ccc5c3c24)c2ccc3oc4ccccc4c3c2)cc1]

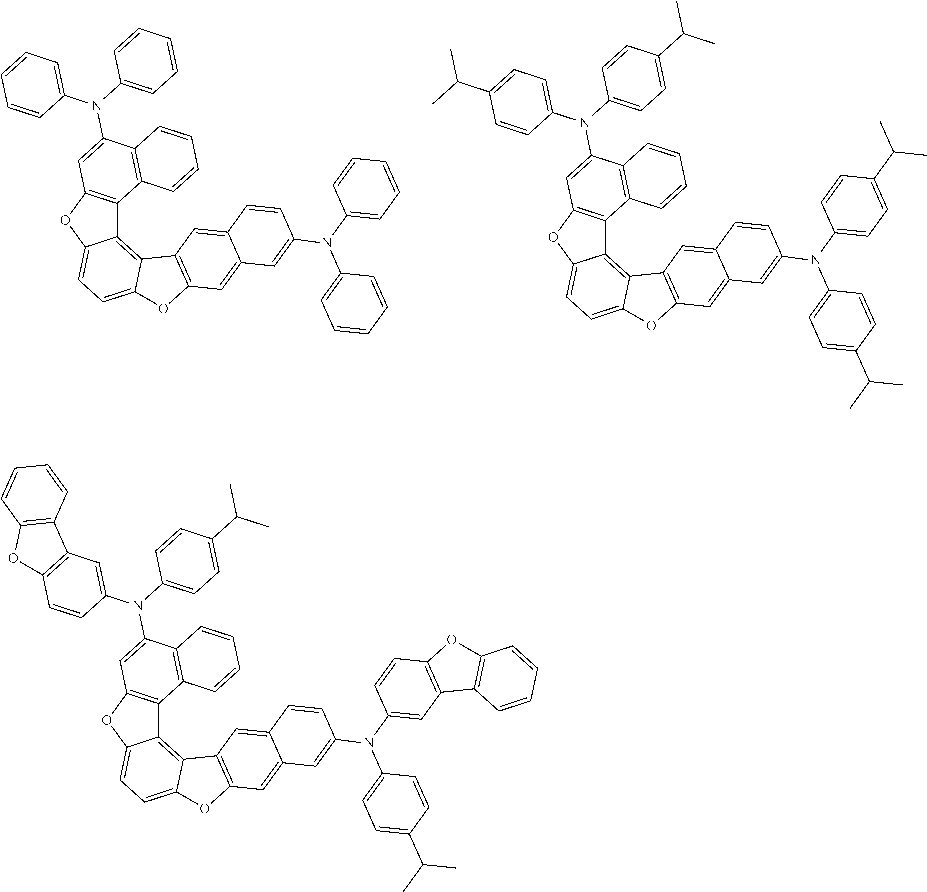 [SMILES: c1ccc(N(c2ccccc2)c2ccc3cc4c(cc3c2)oc2ccc3oc5cc(N(c6ccccc6)c6ccccc6)c6ccccc6c5c3c24)cc1 ; CC(C)c1ccc(N(c2ccc(C(C)C)cc2)c2ccc3cc4c(cc3c2)oc2ccc3oc5cc(N(c6ccc(C(C)C)cc6)c6ccc(C(C)C)cc6)c6ccccc6c5c3c24)cc1 ; CC(C)c1ccc(N(c2ccc3cc4c(cc3c2)oc2ccc3oc5cc(N(c6ccc(C(C)C)cc6)c6ccc7oc8ccccc8c7c6)c6ccccc6c5c3c24)c2ccc3oc4ccccc4c3c2)cc1]

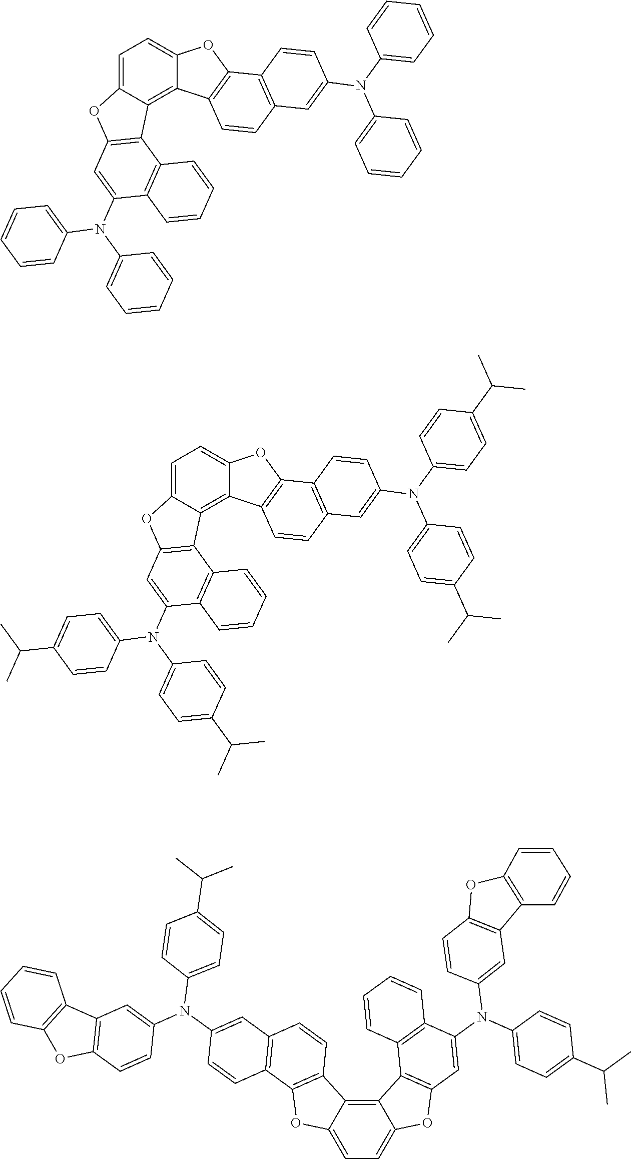 [SMILES: c1ccc(N(c2ccccc2)c2ccc3c(ccc4c3oc3ccc5oc6cc(N(c7ccccc7)c7ccccc7)c7ccccc7c6c5c34)c2)cc1 ; CC(C)c1ccc(N(c2ccc(C(C)C)cc2)c2ccc3c(ccc4c3oc3ccc5oc6cc(N(c7ccc(C(C)C)cc7)c7ccc(C(C)C)cc7)c7ccccc7c6c5c34)c2)cc1 ; CC(C)c1ccc(N(c2ccc3c(ccc4c3oc3ccc5oc6cc(N(c7ccc(C(C)C)cc7)c7ccc8oc9ccccc9c8c7)c7ccccc7c6c5c34)c2)c2ccc3oc4ccccc4c3c2)cc1]

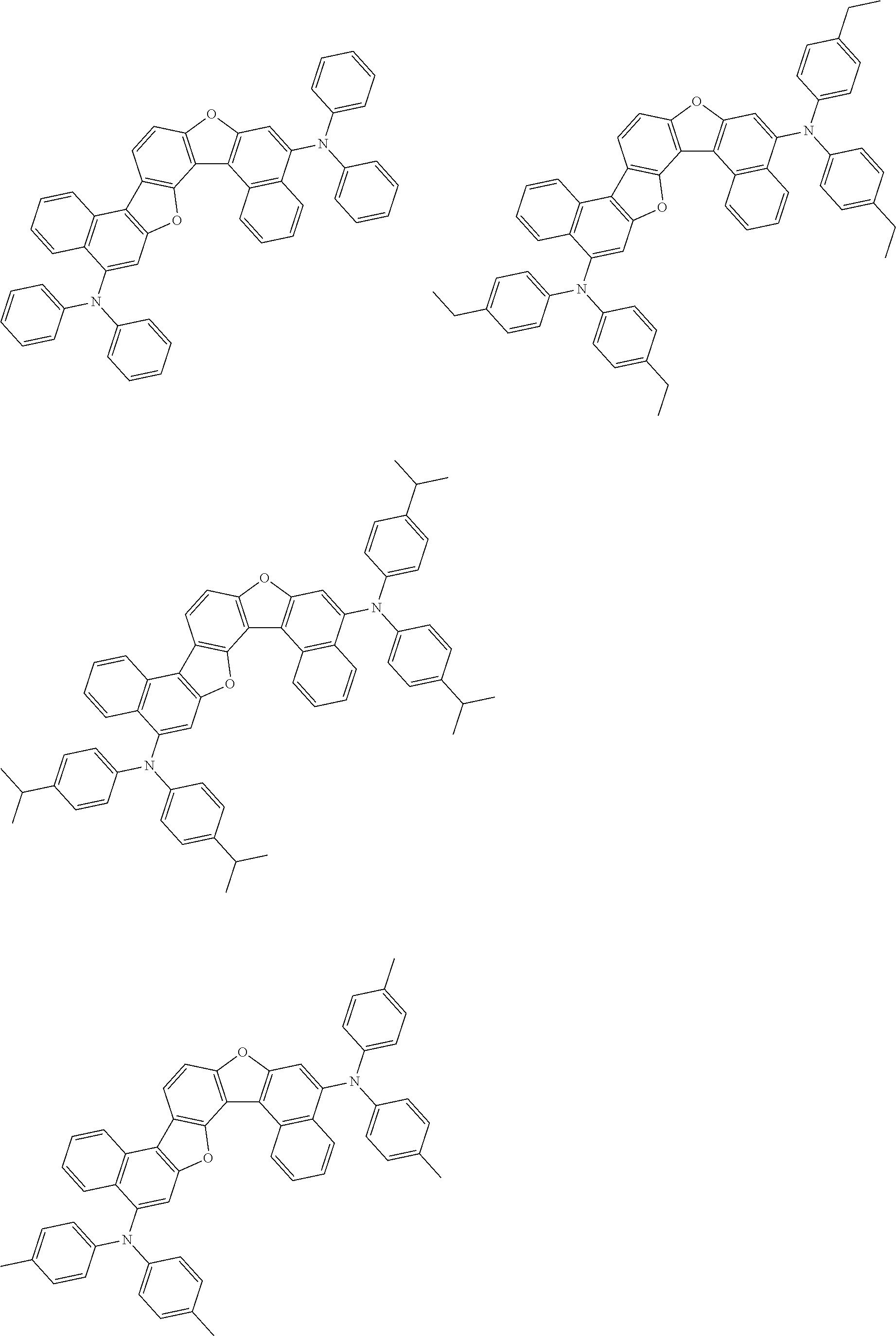

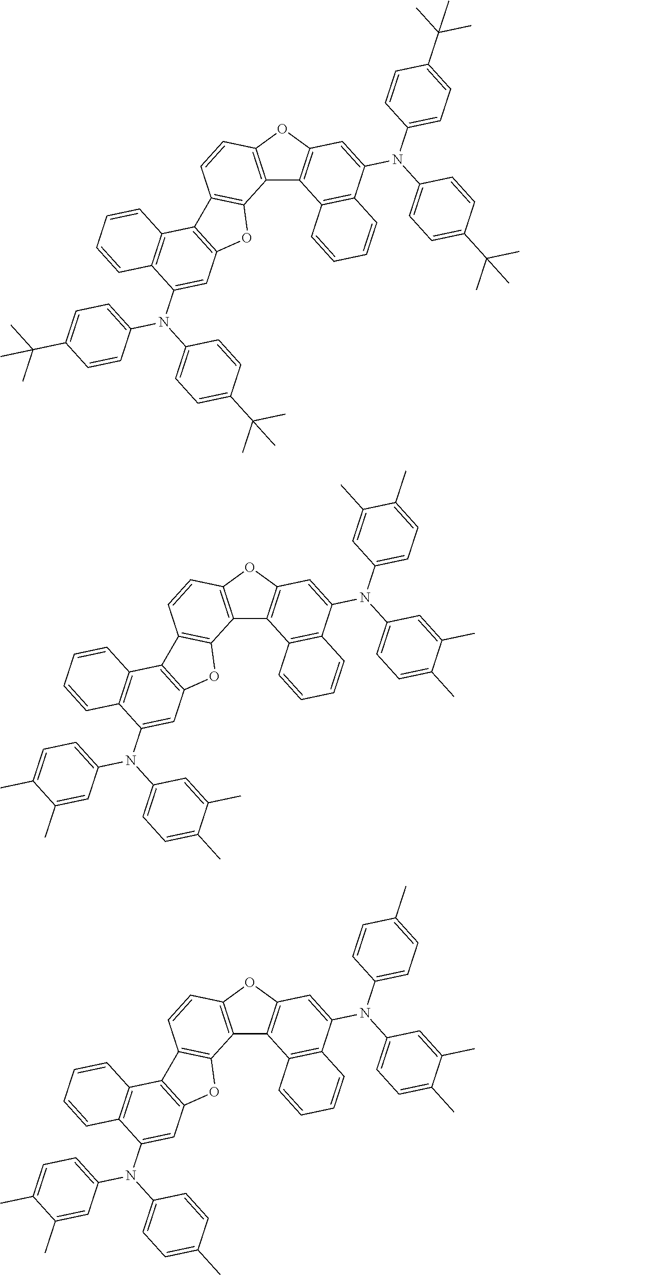

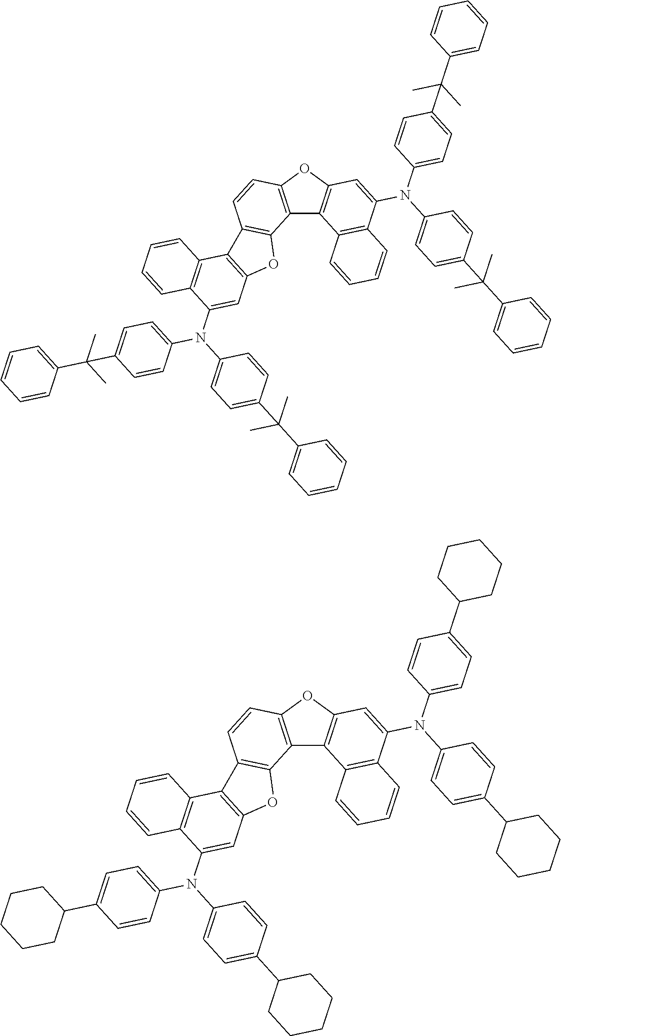 [SMILES: CC(C)(c1ccccc1)c1ccc(N(c2ccc(C(C)(C)c3ccccc3)cc2)c2cc3oc4c(ccc5oc6cc(N(c7ccc(C(C)(C)c8ccccc8)cc7)c7ccc(C(C)(C)c8ccccc8)cc7)c7ccccc7c6c54)c3c3ccccc23)cc1 ; c1ccc2c(c1)c(N(c1ccc(C3CCCCC3)cc1)c1ccc(C3CCCCC3)cc1)cc1oc3c(ccc4oc5cc(N(c6ccc(C7CCCCC7)cc6)c6ccc(C7CCCCC7)cc6)c6ccccc6c5c43)c12]

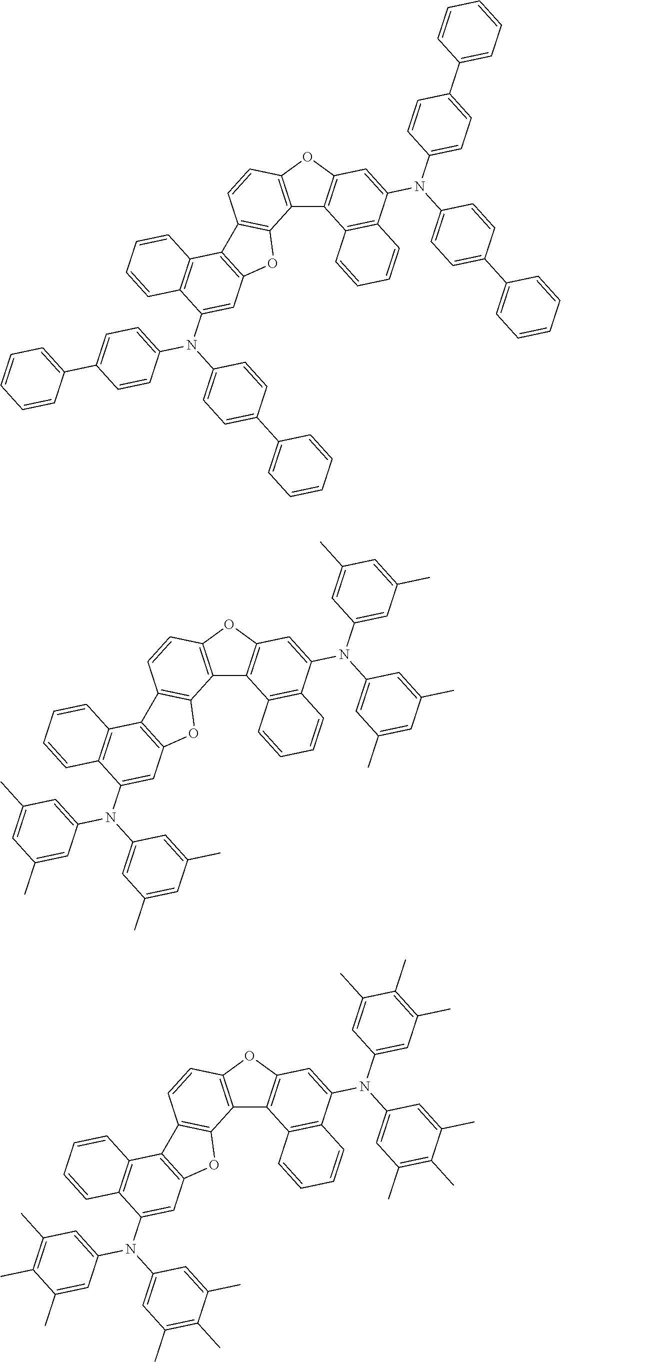

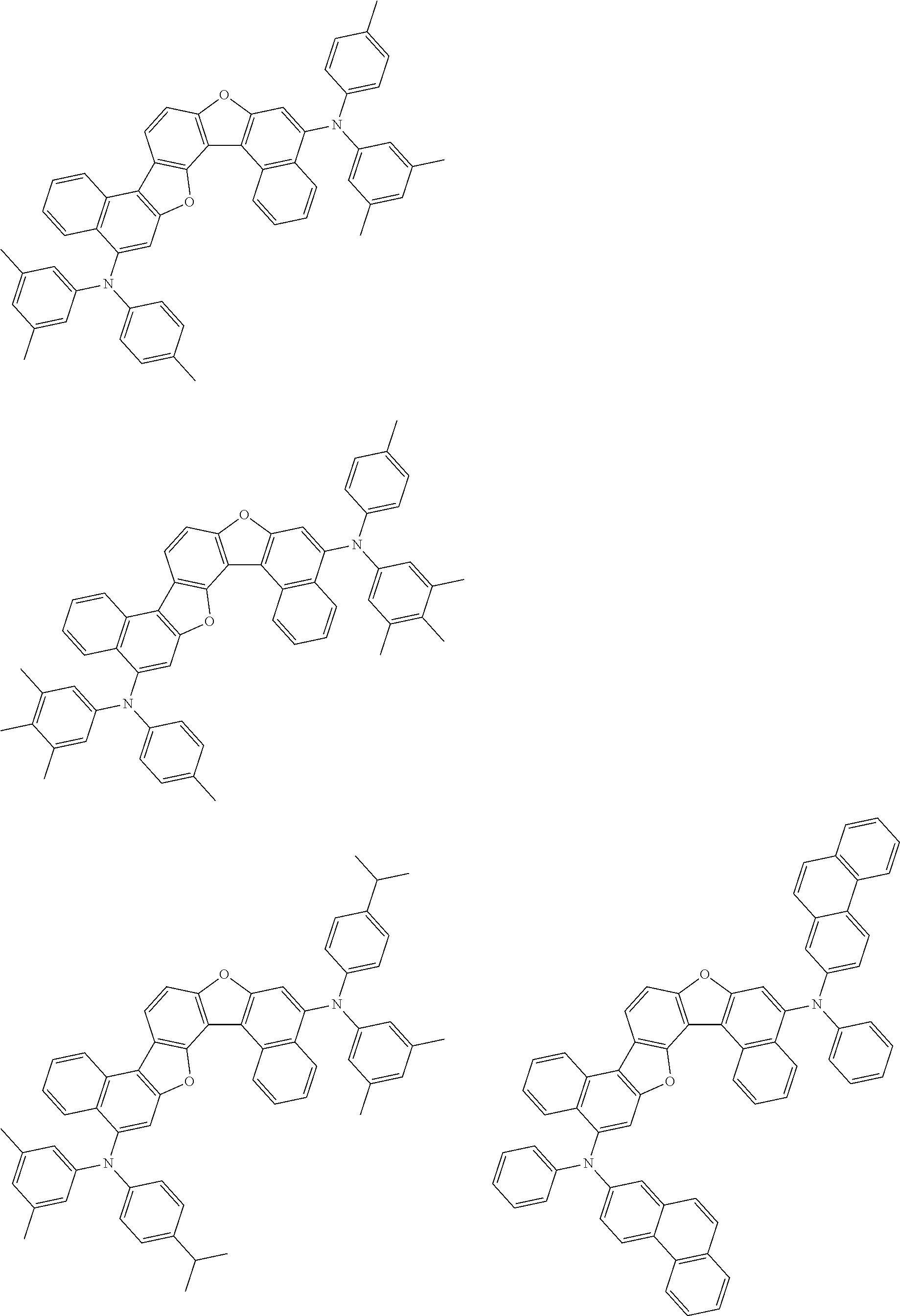

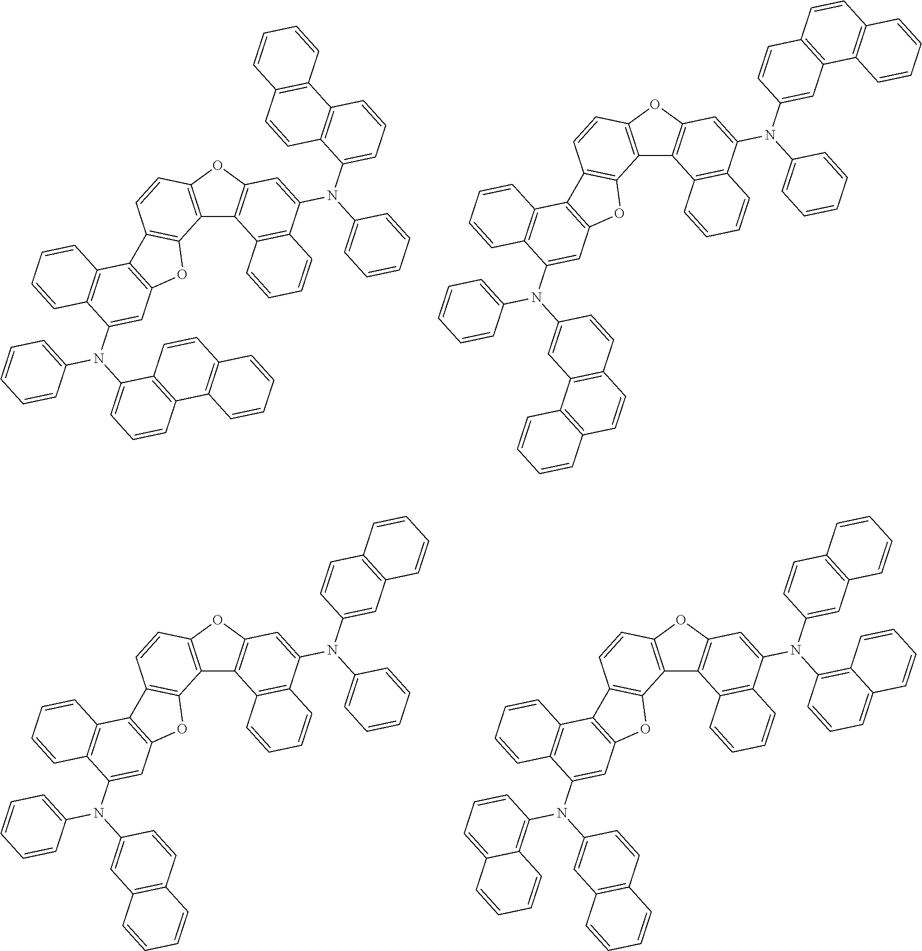 [SMILES: c1ccc(N(c2cccc3c2ccc2ccccc23)c2cc3oc4c(ccc5oc6cc(N(c7ccccc7)c7cccc8c7ccc7ccccc78)c7ccccc7c6c54)c3c3ccccc23)cc1 ; c1ccc(N(c2ccc3ccc4ccccc4c3c2)c2cc3oc4c(ccc5oc6cc(N(c7ccccc7)c7ccc8ccc9ccccc9c8c7)c7ccccc7c6c54)c3c3ccccc23)cc1 ; c1ccc(N(c2ccc3ccccc3c2)c2cc3oc4c(ccc5oc6cc(N(c7ccccc7)c7ccc8ccccc8c7)c7ccccc7c6c54)c3c3ccccc23)cc1 ; c1ccc2cc(N(c3cccc4ccccc34)c3cc4oc5c(ccc6oc7cc(N(c8ccc9ccccc9c8)c8cccc9ccccc89)c8ccccc8c7c65)c4c4ccccc34)ccc2c1]

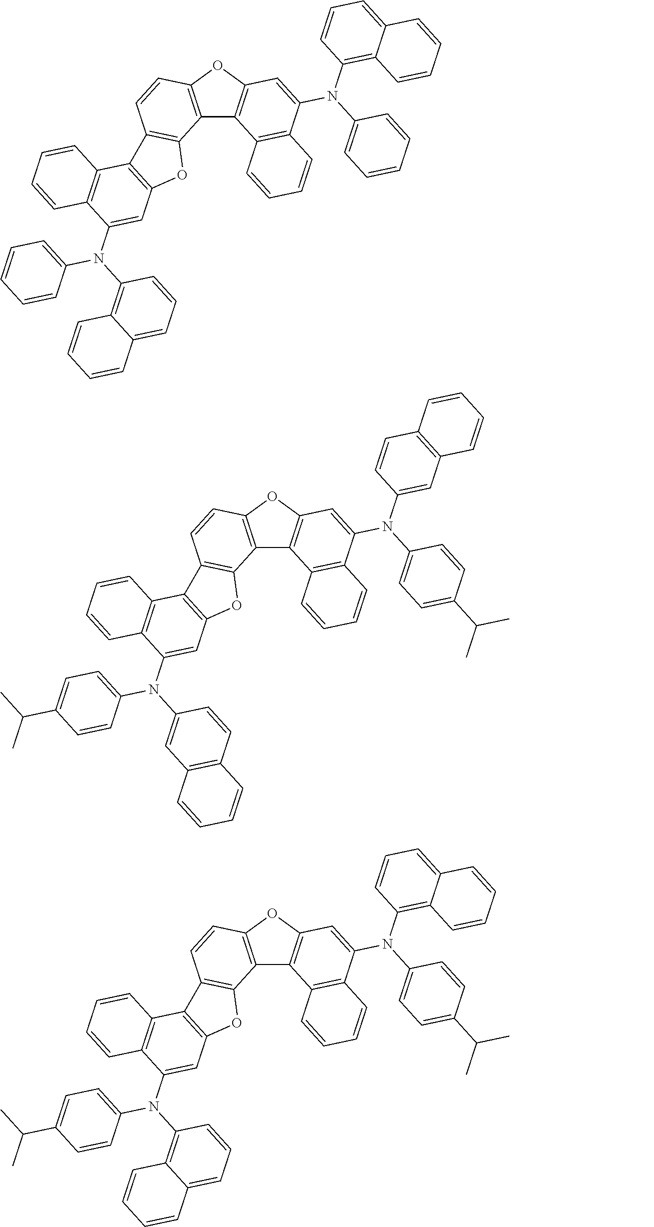

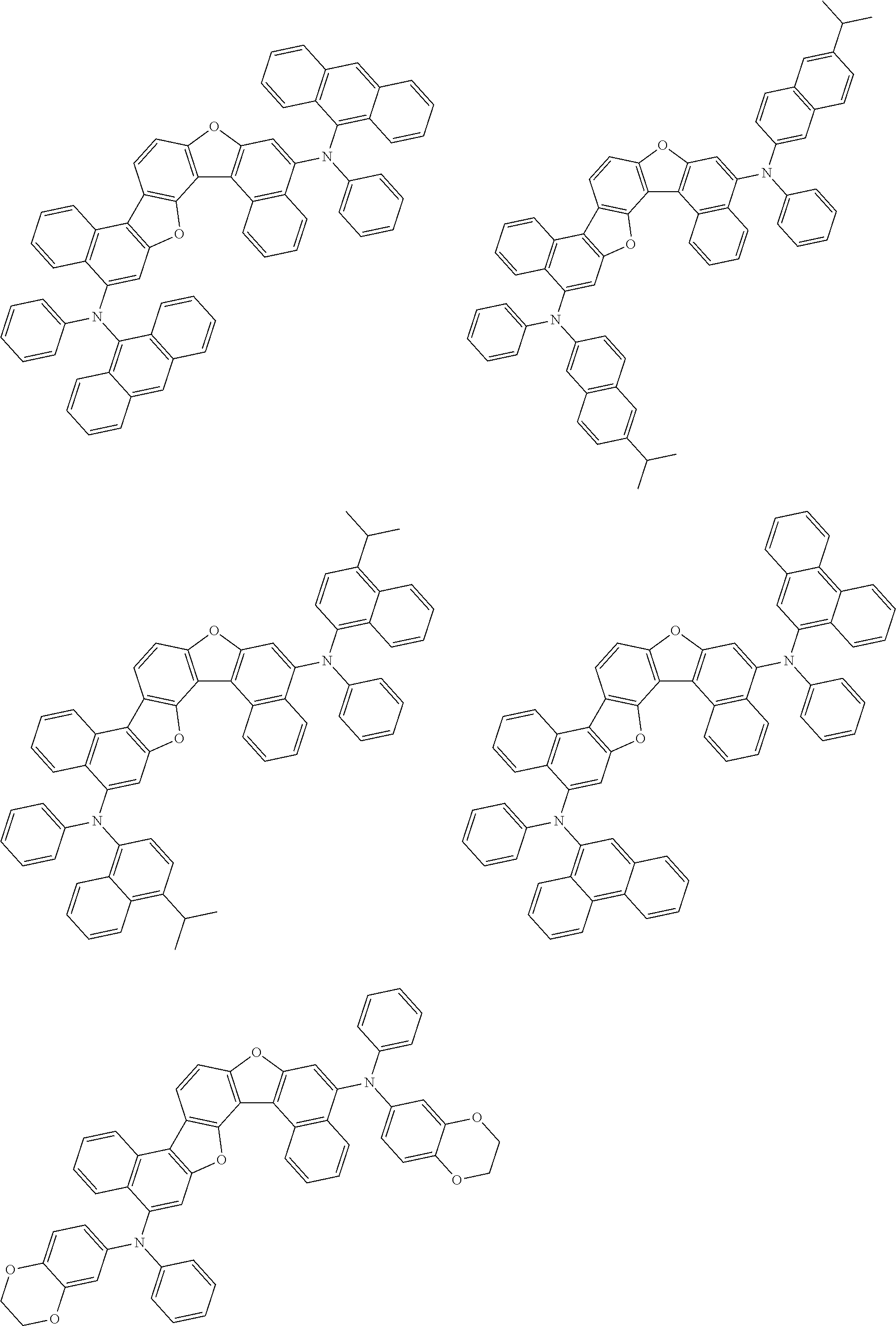

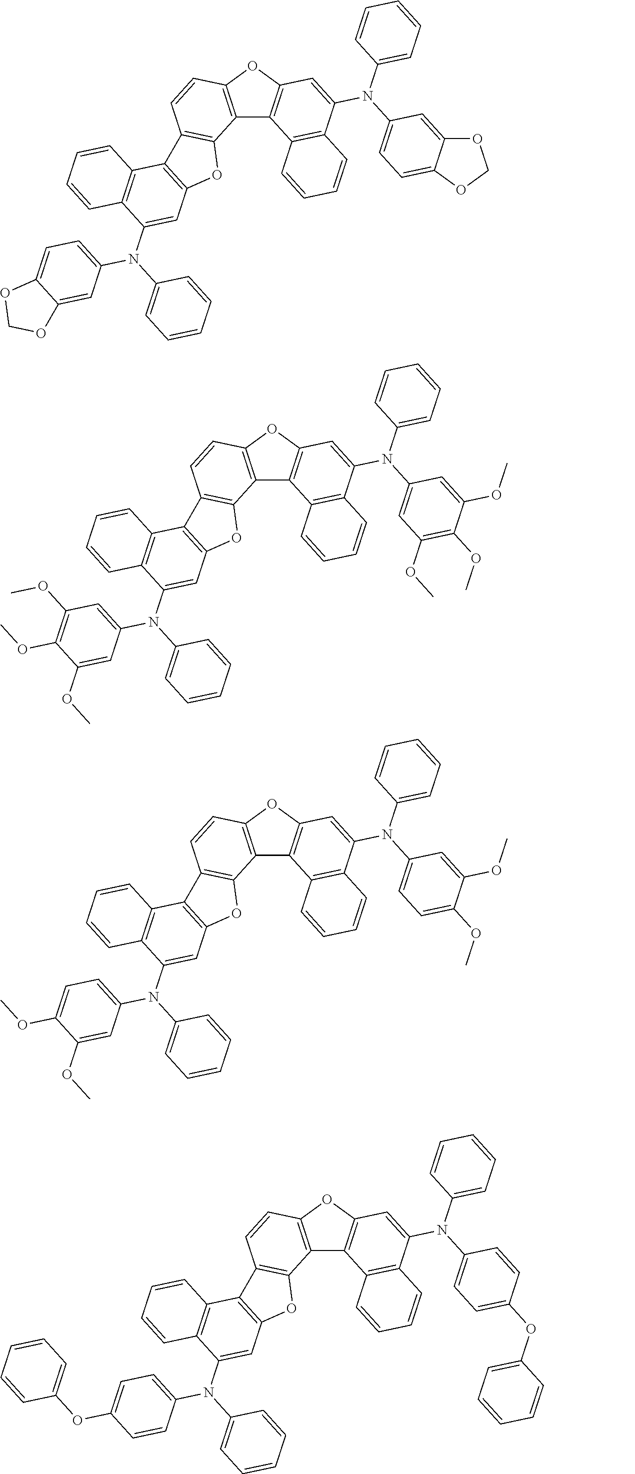

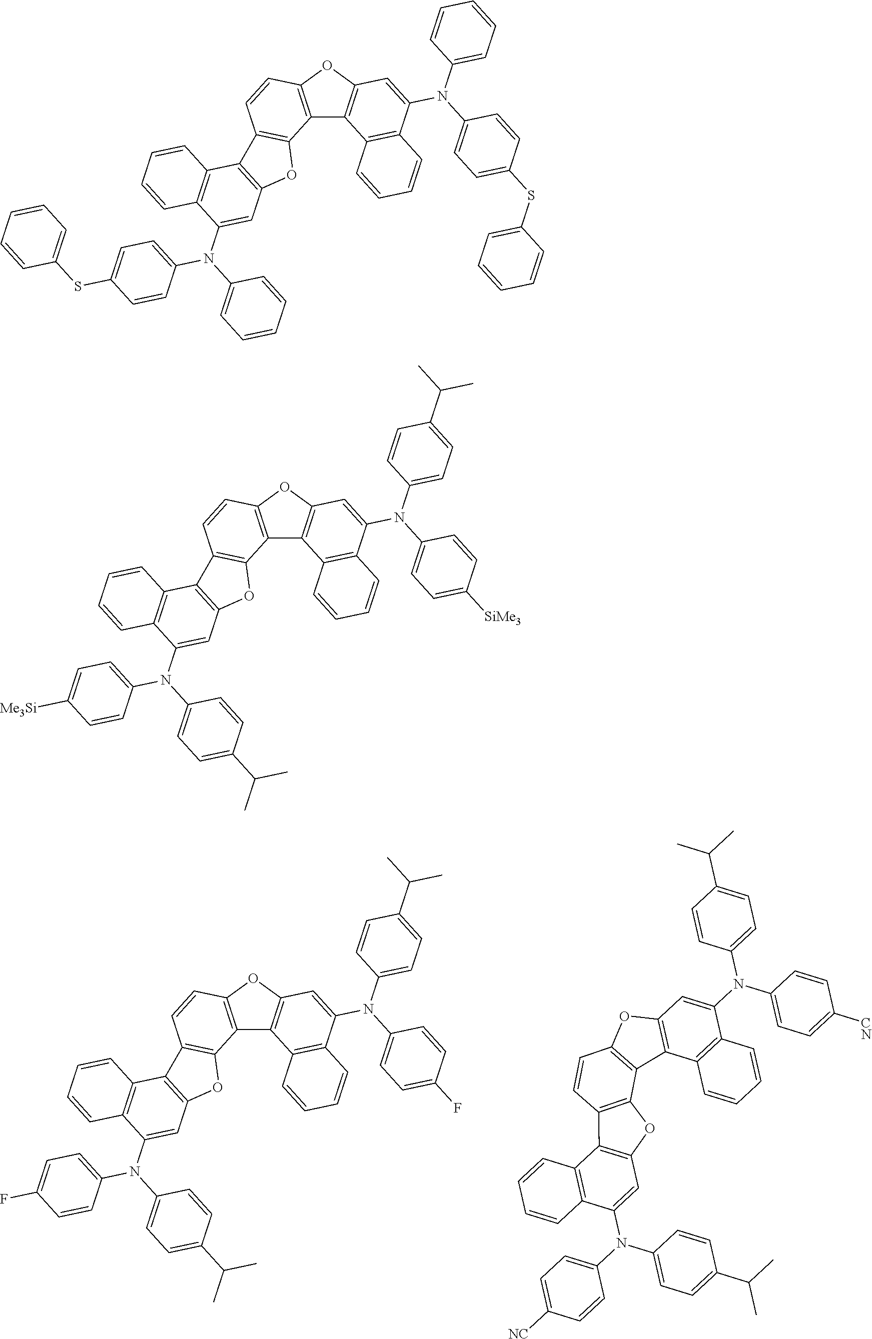 [SMILES: c1ccc(Sc2ccc(N(c3ccccc3)c3cc4oc5c(ccc6oc7cc(N(c8ccccc8)c8ccc(Sc9ccccc9)cc8)c8ccccc8c7c65)c4c4ccccc34)cc2)cc1 ; CC(C)c1ccc(N(c2ccc([Si](C)(C)C)cc2)c2cc3oc4c(ccc5oc6cc(N(c7ccc(C(C)C)cc7)c7ccc([Si](C)(C)C)cc7)c7ccccc7c6c54)c3c3ccccc23)cc1 ; CC(C)c1ccc(N(c2ccc(F)cc2)c2cc3oc4c(ccc5oc6cc(N(c7ccc(F)cc7)c7ccc(C(C)C)cc7)c7ccccc7c6c54)c3c3ccccc23)cc1 ; CC(C)c1ccc(N(c2ccc(C#N)cc2)c2cc3oc4c(ccc5oc6cc(N(c7ccc(C#N)cc7)c7ccc(C(C)C)cc7)c7ccccc7c6c54)c3c3ccccc23)cc1]

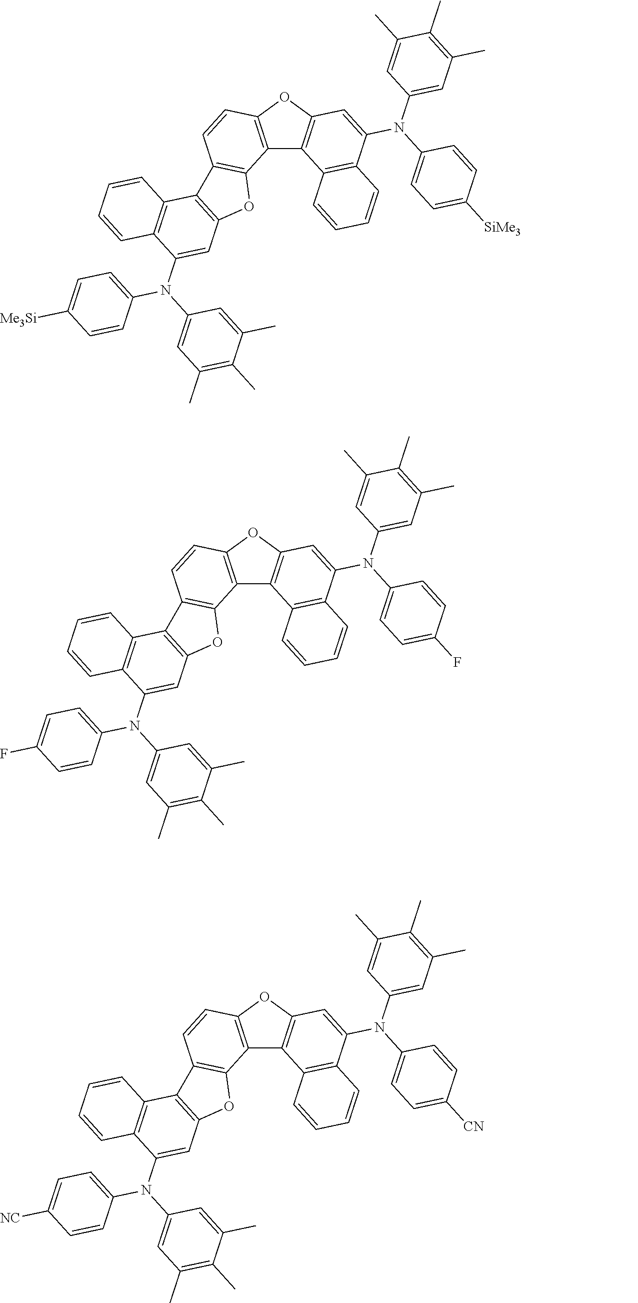 [SMILES: Cc1cc(N(c2ccc([Si](C)(C)C)cc2)c2cc3oc4c(ccc5oc6cc(N(c7ccc([Si](C)(C)C)cc7)c7cc(C)c(C)c(C)c7)c7ccccc7c6c54)c3c3ccccc23)cc(C)c1C ; Cc1cc(N(c2ccc(F)cc2)c2cc3oc4c(ccc5oc6cc(N(c7ccc(F)cc7)c7cc(C)c(C)c(C)c7)c7ccccc7c6c54)c3c3ccccc23)cc(C)c1C ; Cc1cc(N(c2ccc(C#N)cc2)c2cc3oc4c(ccc5oc6cc(N(c7ccc(C#N)cc7)c7cc(C)c(C)c(C)c7)c7ccccc7c6c54)c3c3ccccc23)cc(C)c1C]

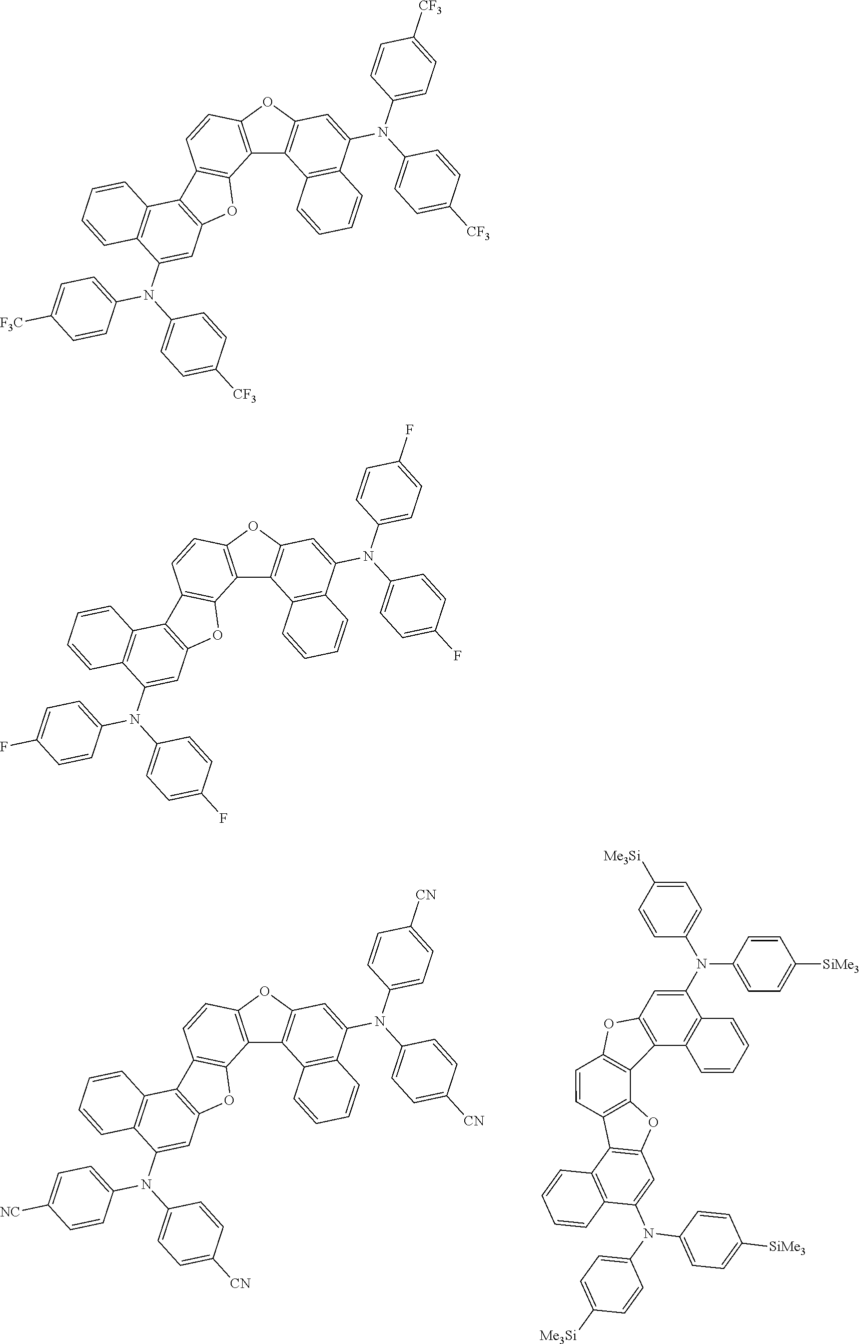 [SMILES: FC(F)(F)c1ccc(N(c2ccc(C(F)(F)F)cc2)c2cc3oc4c(ccc5oc6cc(N(c7ccc(C(F)(F)F)cc7)c7ccc(C(F)(F)F)cc7)c7ccccc7c6c54)c3c3ccccc23)cc1 ; Fc1ccc(N(c2ccc(F)cc2)c2cc3oc4c(ccc5oc6cc(N(c7ccc(F)cc7)c7ccc(F)cc7)c7ccccc7c6c54)c3c3ccccc23)cc1 ; N#Cc1ccc(N(c2ccc(C#N)cc2)c2cc3oc4c(ccc5oc6cc(N(c7ccc(C#N)cc7)c7ccc(C#N)cc7)c7ccccc7c6c54)c3c3ccccc23)cc1 ; C[Si](C)(C)c1ccc(N(c2ccc([Si](C)(C)C)cc2)c2cc3oc4c(ccc5oc6cc(N(c7ccc([Si](C)(C)C)cc7)c7ccc([Si](C)(C)C)cc7)c7ccccc7c6c54)c3c3ccccc23)cc1]

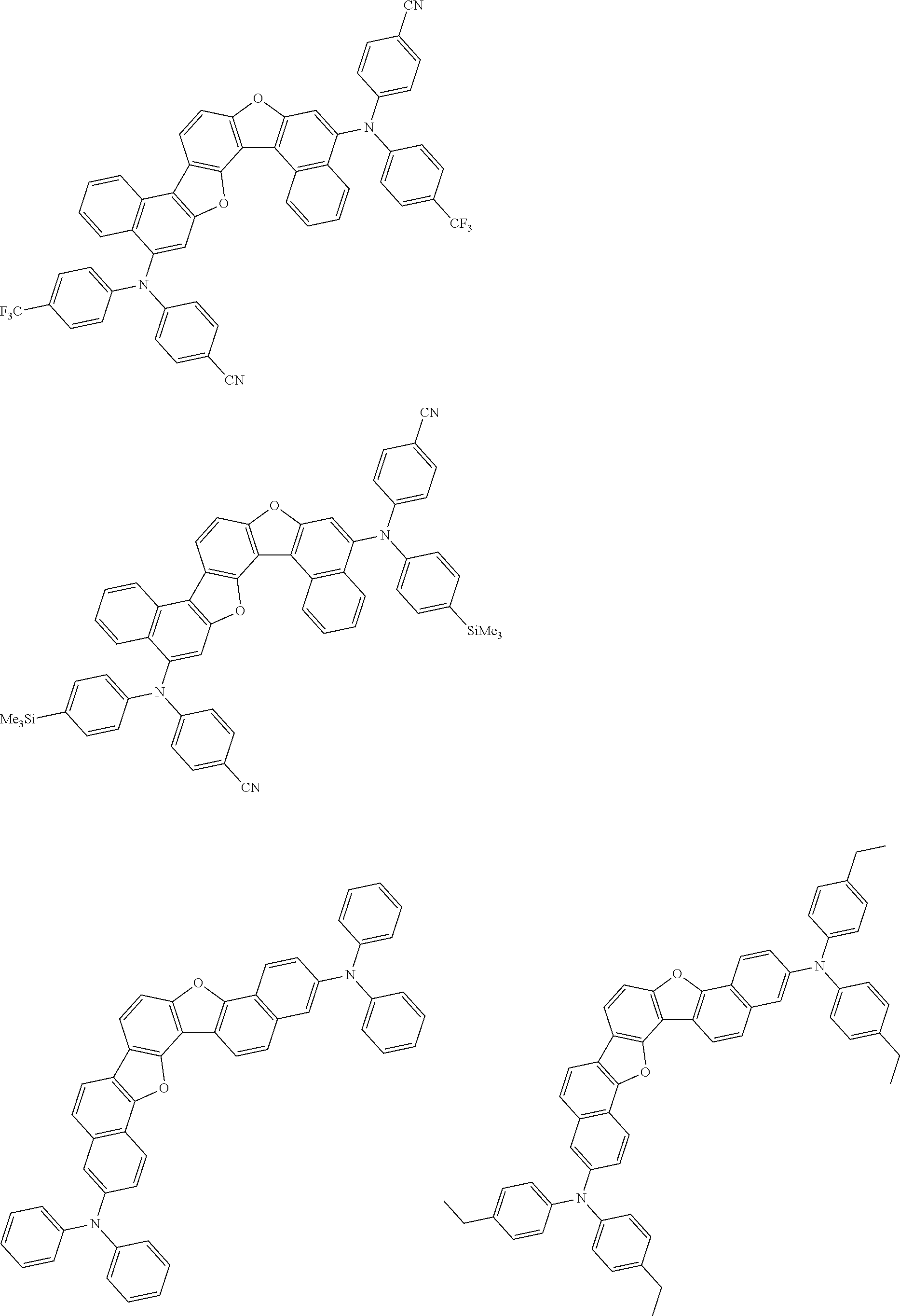 [SMILES: N#Cc1ccc(N(c2ccc(C(F)(F)F)cc2)c2cc3oc4c(ccc5oc6cc(N(c7ccc(C#N)cc7)c7ccc(C(F)(F)F)cc7)c7ccccc7c6c54)c3c3ccccc23)cc1 ; C[Si](C)(C)c1ccc(N(c2ccc(C#N)cc2)c2cc3oc4c(ccc5oc6cc(N(c7ccc(C#N)cc7)c7ccc([Si](C)(C)C)cc7)c7ccccc7c6c54)c3c3ccccc23)cc1 ; c1ccc(N(c2ccccc2)c2ccc3c(ccc4c5ccc6oc7c8ccc(N(c9ccccc9)c9ccccc9)cc8ccc7c6c5oc34)c2)cc1 ; CCc1ccc(N(c2ccc(CC)cc2)c2ccc3c(ccc4c5ccc6oc7c8ccc(N(c9ccc(CC)cc9)c9ccc(CC)cc9)cc8ccc7c6c5oc34)c2)cc1]

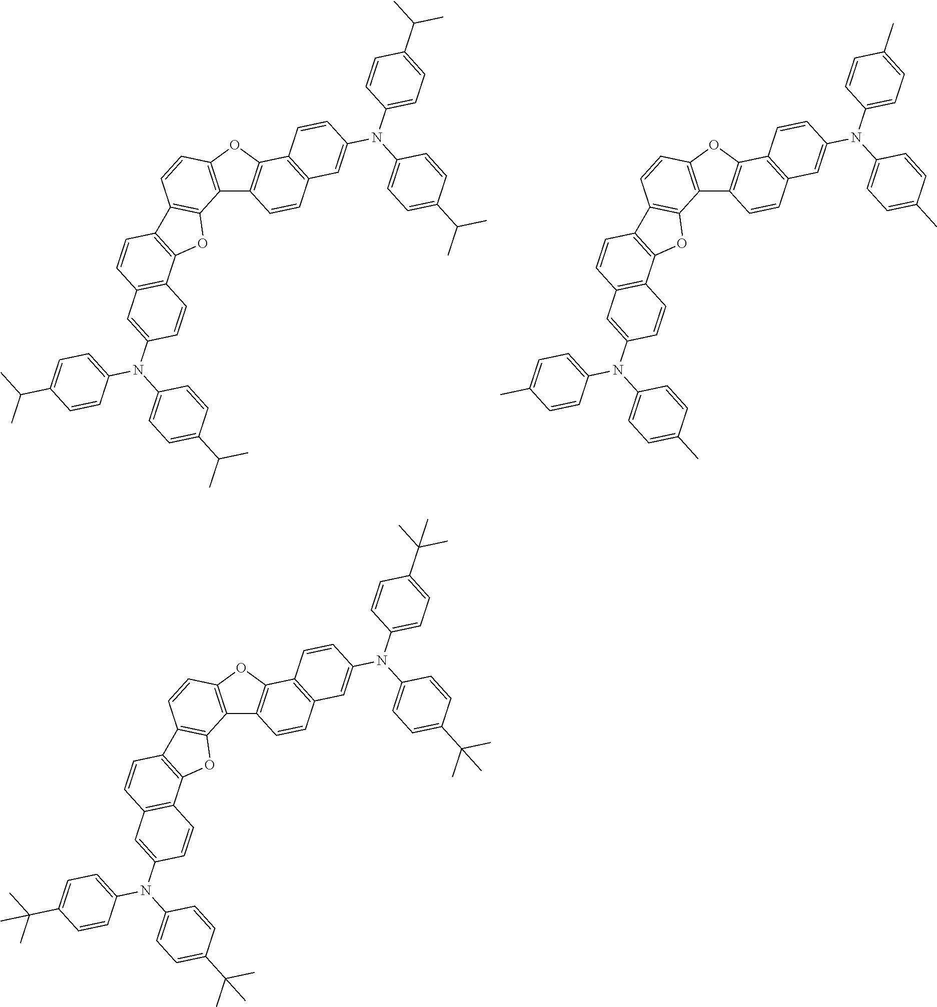

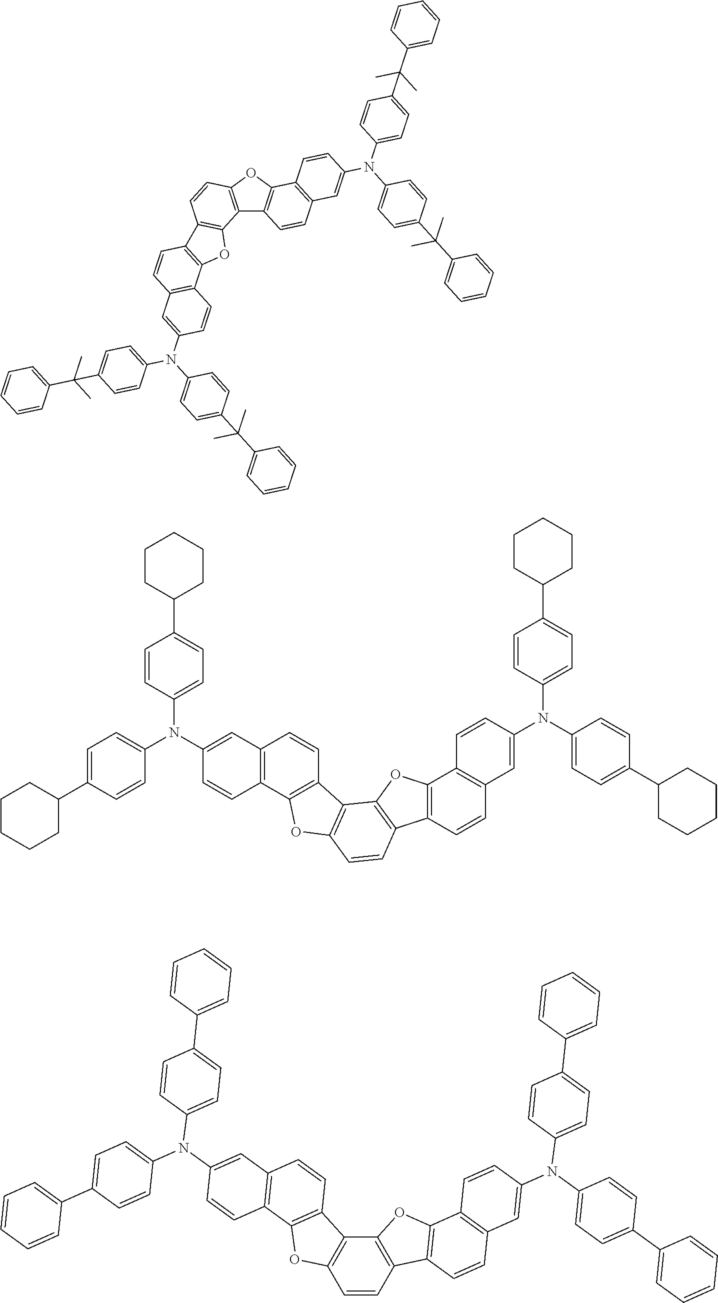 [SMILES: CC(C)(c1ccccc1)c1ccc(N(c2ccc(C(C)(C)c3ccccc3)cc2)c2ccc3c(ccc4c5ccc6oc7c8ccc(N(c9ccc(C(C)(C)c%10ccccc%10)cc9)c9ccc(C(C)(C)c%10ccccc%10)cc9)cc8ccc7c6c5oc34)c2)cc1 ; c1cc(N(c2ccc(C3CCCCC3)cc2)c2ccc3c(ccc4c5ccc6oc7c8ccc(N(c9ccc(C%10CCCCC%10)cc9)c9ccc(C%10CCCCC%10)cc9)cc8ccc7c6c5oc34)c2)ccc1C1CCCCC1 ; c1ccc(-c2ccc(N(c3ccc(-c4ccccc4)cc3)c3ccc4c(ccc5c6ccc7oc8c9ccc(N(c%10ccc(-c%11ccccc%11)cc%10)c%10ccc(-c%11ccccc%11)cc%10)cc9ccc8c7c6oc45)c3)cc2)cc1]

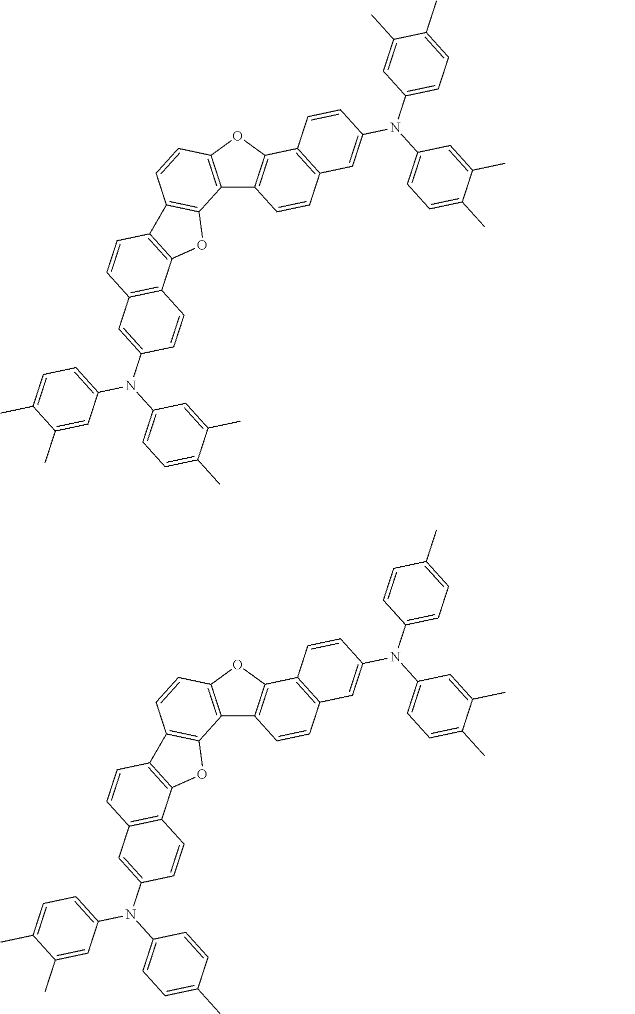

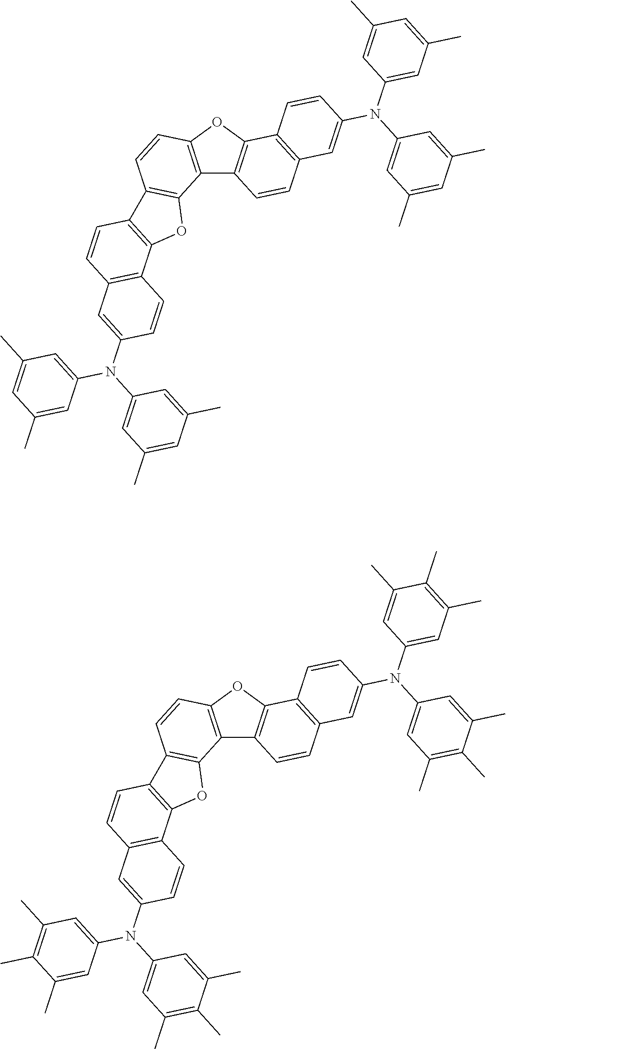

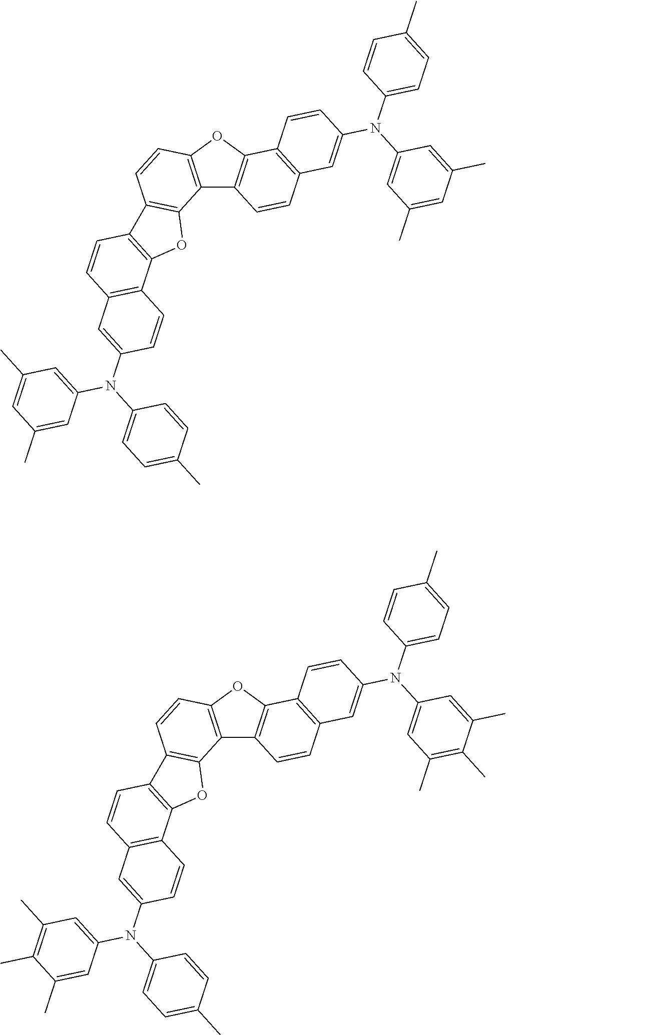

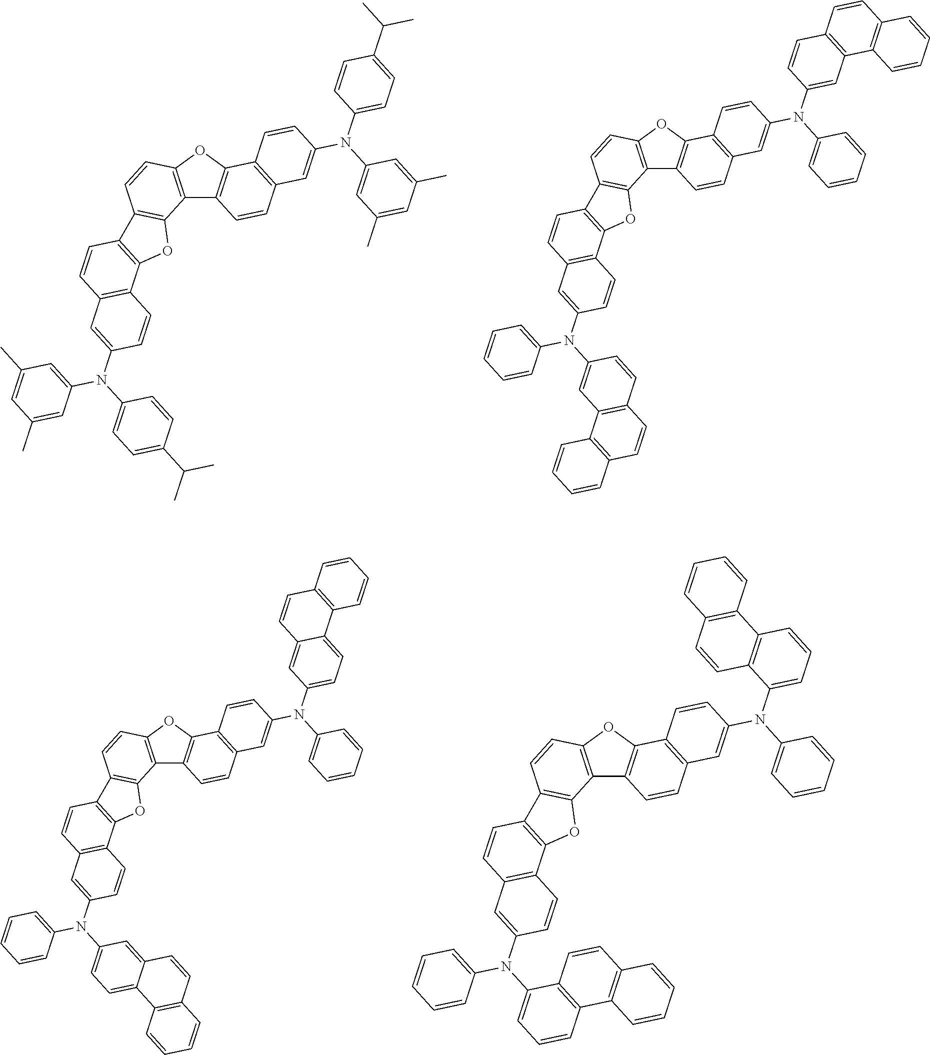

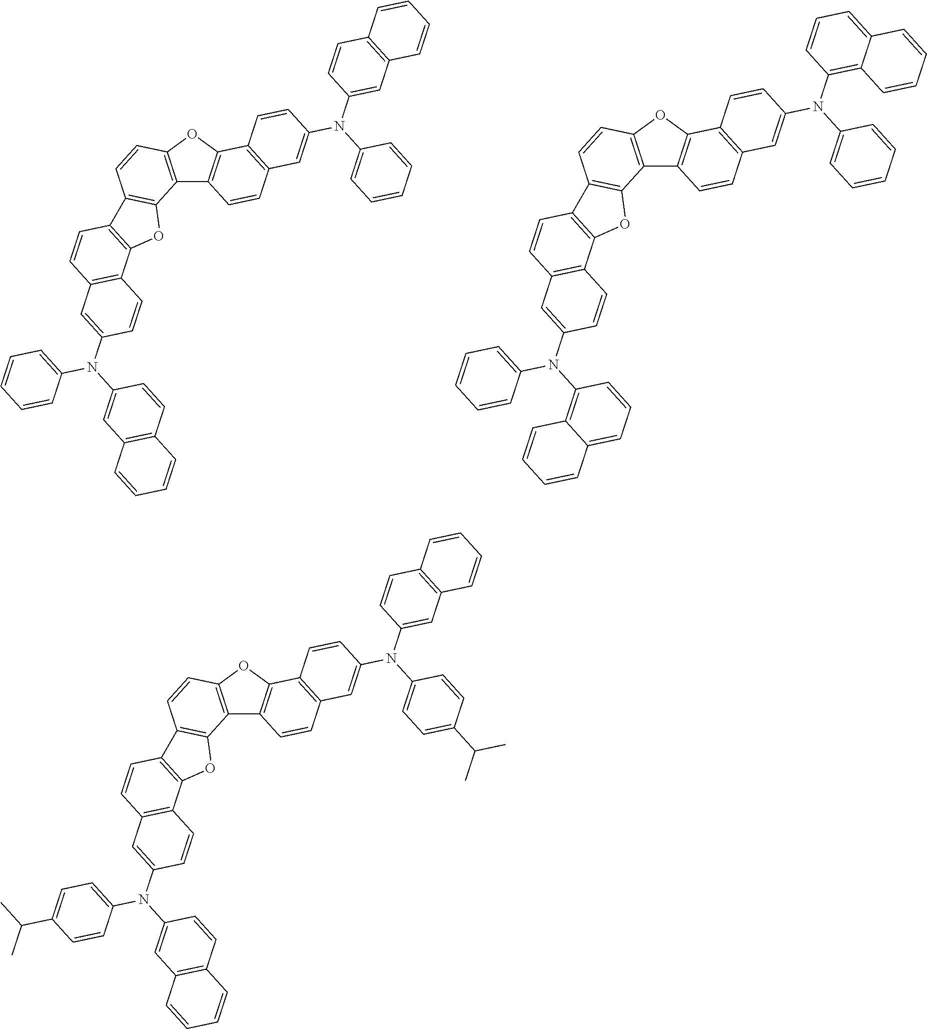 [SMILES: c1ccc(N(c2ccc3ccccc3c2)c2ccc3c(ccc4c5ccc6oc7c8ccc(N(c9ccccc9)c9ccc%10ccccc%10c9)cc8ccc7c6c5oc34)c2)cc1 ; c1ccc(N(c2ccc3c(ccc4c5ccc6oc7c8ccc(N(c9ccccc9)c9cccc%10ccccc9%10)cc8ccc7c6c5oc34)c2)c2cccc3ccccc23)cc1 ; CC(C)c1ccc(N(c2ccc3ccccc3c2)c2ccc3c(ccc4c5ccc6oc7c8ccc(N(c9ccc(C(C)C)cc9)c9ccc%10ccccc%10c9)cc8ccc7c6c5oc34)c2)cc1]

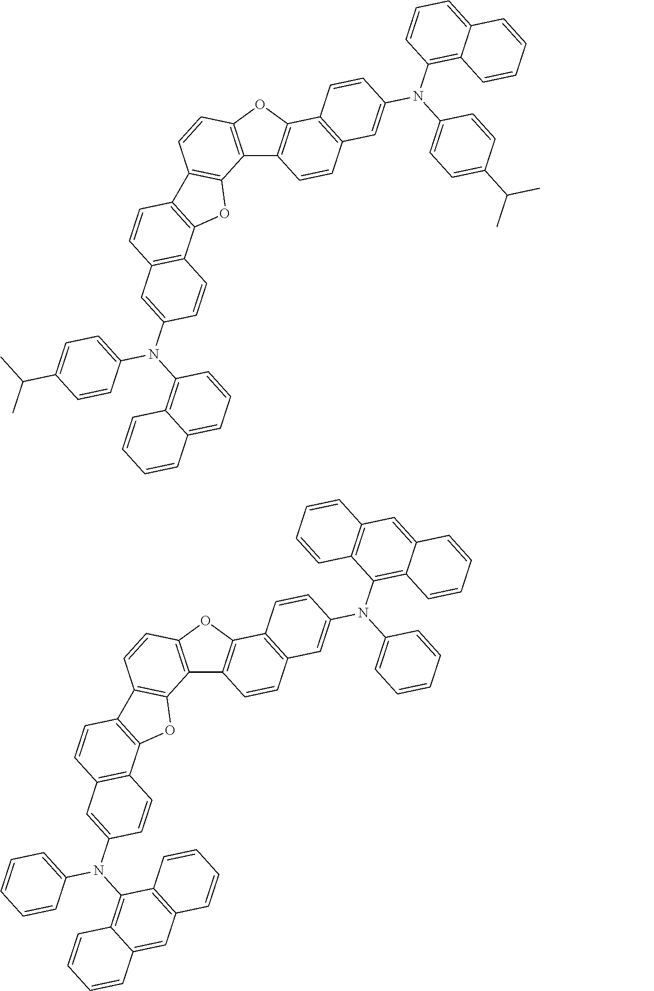 [SMILES: CC(C)c1ccc(N(c2ccc3c(ccc4c5ccc6oc7c8ccc(N(c9ccc(C(C)C)cc9)c9cccc%10ccccc9%10)cc8ccc7c6c5oc34)c2)c2cccc3ccccc23)cc1 ; c1ccc(N(c2ccc3c(ccc4c5ccc6oc7c8ccc(N(c9ccccc9)c9c%10ccccc%10cc%10ccccc9%10)cc8ccc7c6c5oc34)c2)c2c3ccccc3cc3ccccc23)cc1]

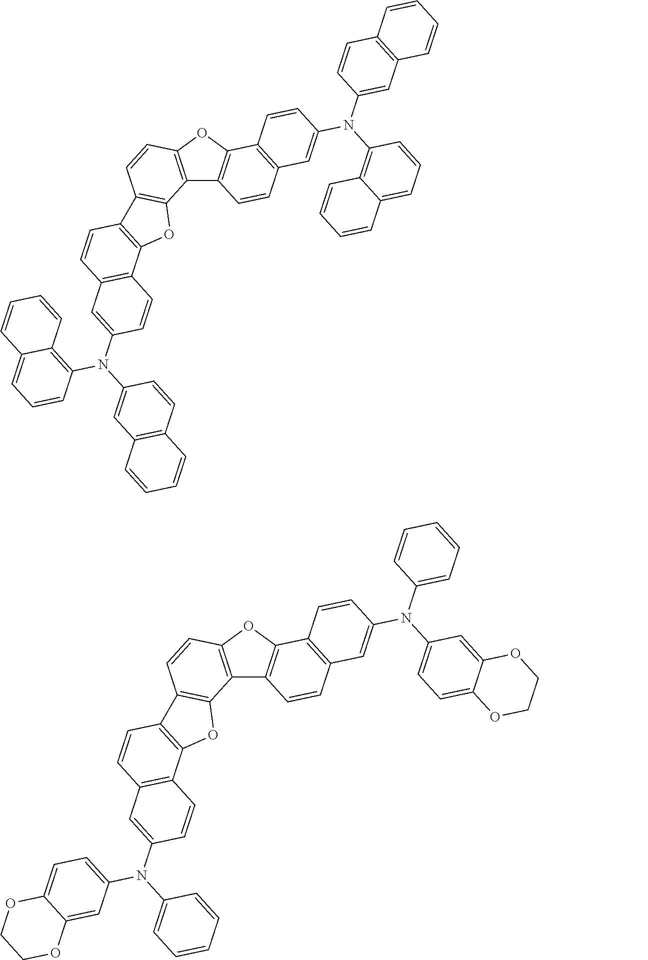 [SMILES: c1ccc2cc(N(c3ccc4c(ccc5c6ccc7oc8c9ccc(N(c%10ccc%11ccccc%11c%10)c%10cccc%11ccccc%10%11)cc9ccc8c7c6oc45)c3)c3cccc4ccccc34)ccc2c1 ; c1ccc(N(c2ccc3c(c2)OCCO3)c2ccc3c(ccc4c5ccc6oc7c8ccc(N(c9ccccc9)c9ccc%10c(c9)OCCO%10)cc8ccc7c6c5oc34)c2)cc1]

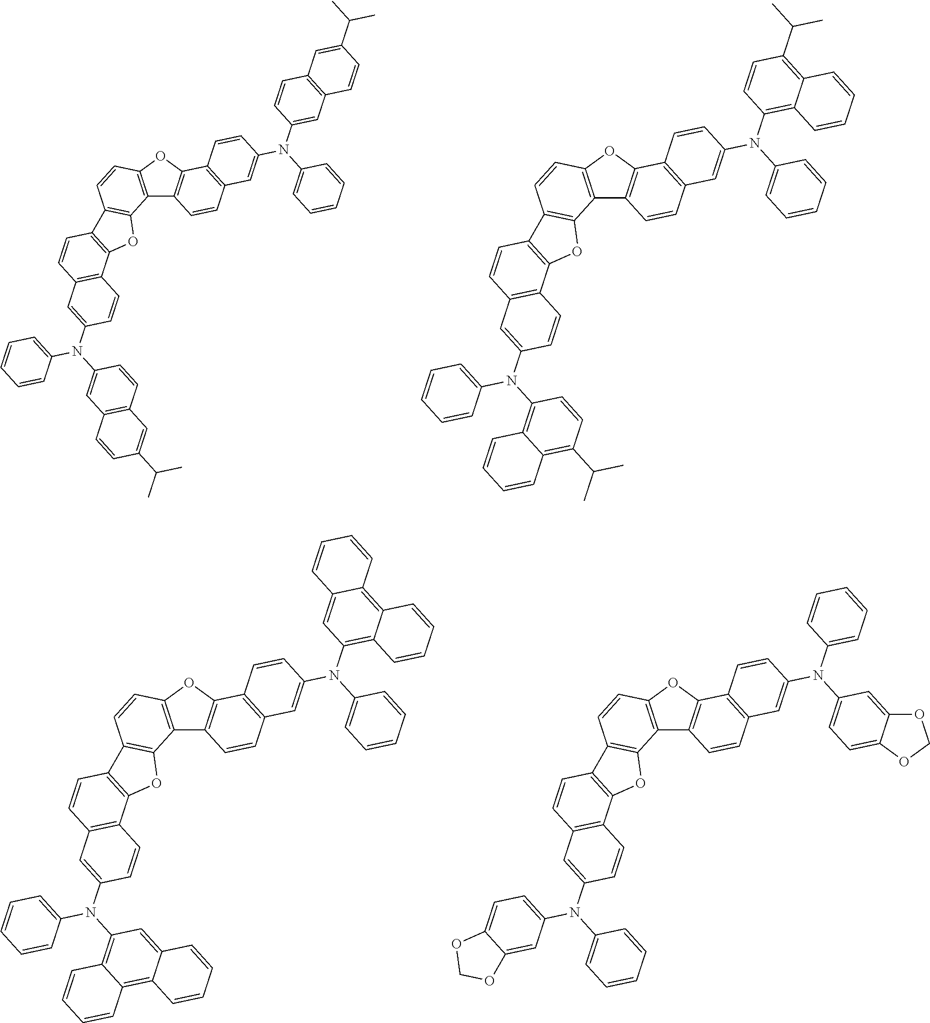 [SMILES: CC(C)c1ccc2cc(N(c3ccccc3)c3ccc4c(ccc5c6ccc7oc8c9ccc(N(c%10ccccc%10)c%10ccc%11cc(C(C)C)ccc%11c%10)cc9ccc8c7c6oc45)c3)ccc2c1 ; CC(C)c1ccc(N(c2ccccc2)c2ccc3c(ccc4c5ccc6oc7c8ccc(N(c9ccccc9)c9ccc(C(C)C)c%10ccccc9%10)cc8ccc7c6c5oc34)c2)c2ccccc12 ; c1ccc(N(c2ccc3c(ccc4c5ccc6oc7c8ccc(N(c9ccccc9)c9cc%10ccccc%10c%10ccccc9%10)cc8ccc7c6c5oc34)c2)c2cc3ccccc3c3ccccc23)cc1 ; c1ccc(N(c2ccc3c(c2)OCO3)c2ccc3c(ccc4c5ccc6oc7c8ccc(N(c9ccccc9)c9ccc%10c(c9)OCO%10)cc8ccc7c6c5oc34)c2)cc1]

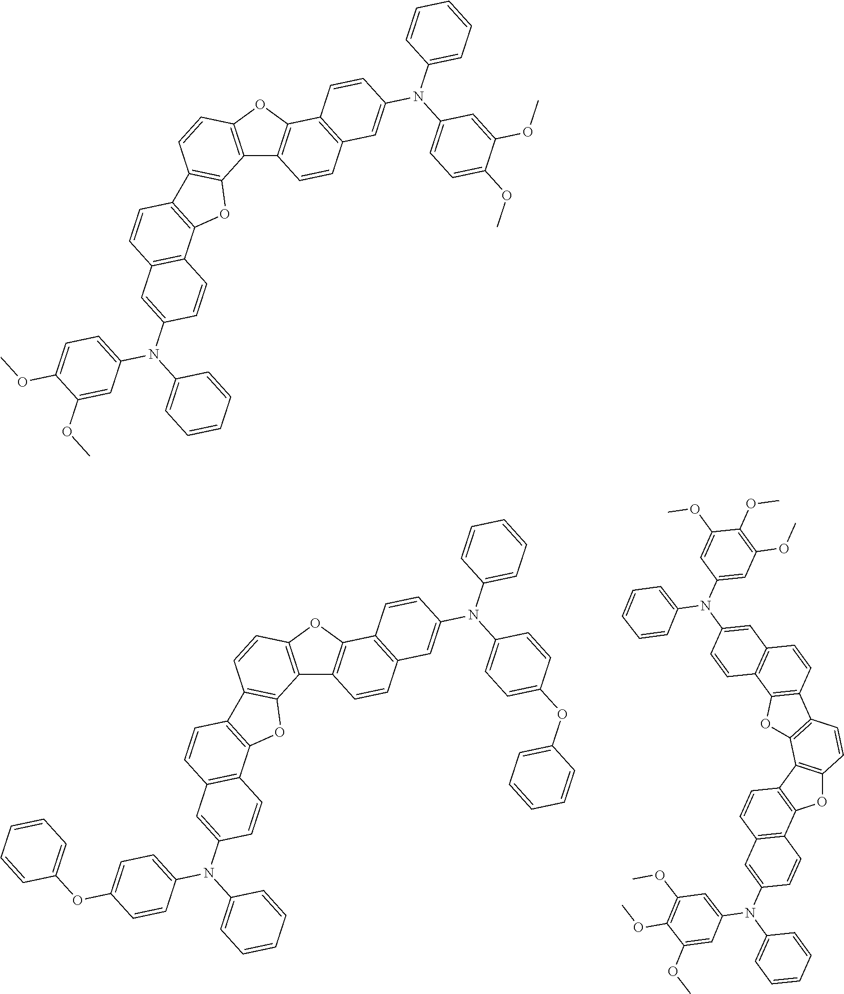

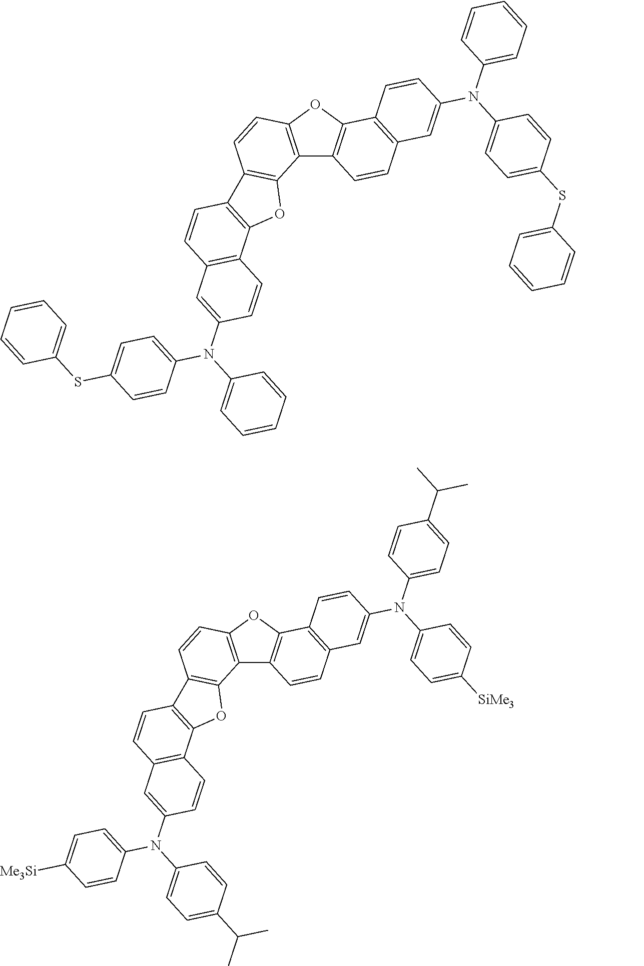 [SMILES: c1ccc(Sc2ccc(N(c3ccccc3)c3ccc4c(ccc5c6ccc7oc8c9ccc(N(c%10ccccc%10)c%10ccc(Sc%11ccccc%11)cc%10)cc9ccc8c7c6oc45)c3)cc2)cc1 ; CC(C)c1ccc(N(c2ccc([Si](C)(C)C)cc2)c2ccc3c(ccc4c5ccc6oc7c8ccc(N(c9ccc(C(C)C)cc9)c9ccc([Si](C)(C)C)cc9)cc8ccc7c6c5oc34)c2)cc1]

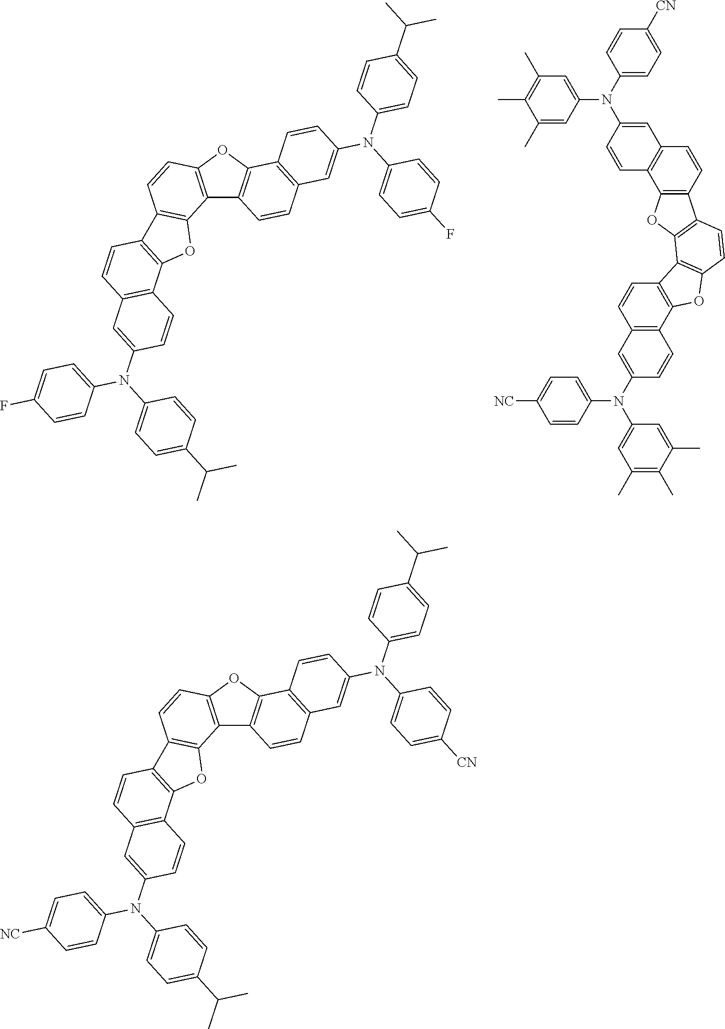 [SMILES: CC(C)c1ccc(N(c2ccc(F)cc2)c2ccc3c(ccc4c5ccc6oc7c8ccc(N(c9ccc(F)cc9)c9ccc(C(C)C)cc9)cc8ccc7c6c5oc34)c2)cc1 ; Cc1cc(N(c2ccc(C#N)cc2)c2ccc3c(ccc4c5ccc6oc7c8ccc(N(c9ccc(C#N)cc9)c9cc(C)c(C)c(C)c9)cc8ccc7c6c5oc34)c2)cc(C)c1C ; CC(C)c1ccc(N(c2ccc(C#N)cc2)c2ccc3c(ccc4c5ccc6oc7c8ccc(N(c9ccc(C#N)cc9)c9ccc(C(C)C)cc9)cc8ccc7c6c5oc34)c2)cc1]

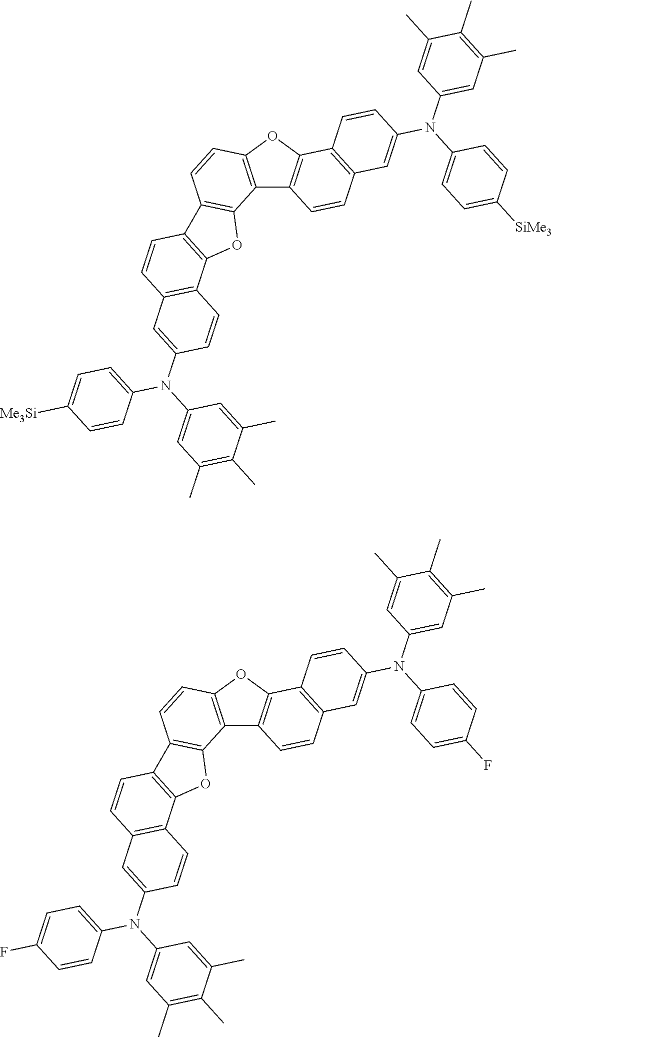 [SMILES: Cc1cc(N(c2ccc([Si](C)(C)C)cc2)c2ccc3c(ccc4c5ccc6oc7c8ccc(N(c9ccc([Si](C)(C)C)cc9)c9cc(C)c(C)c(C)c9)cc8ccc7c6c5oc34)c2)cc(C)c1C ; Cc1cc(N(c2ccc(F)cc2)c2ccc3c(ccc4c5ccc6oc7c8ccc(N(c9ccc(F)cc9)c9cc(C)c(C)c(C)c9)cc8ccc7c6c5oc34)c2)cc(C)c1C]

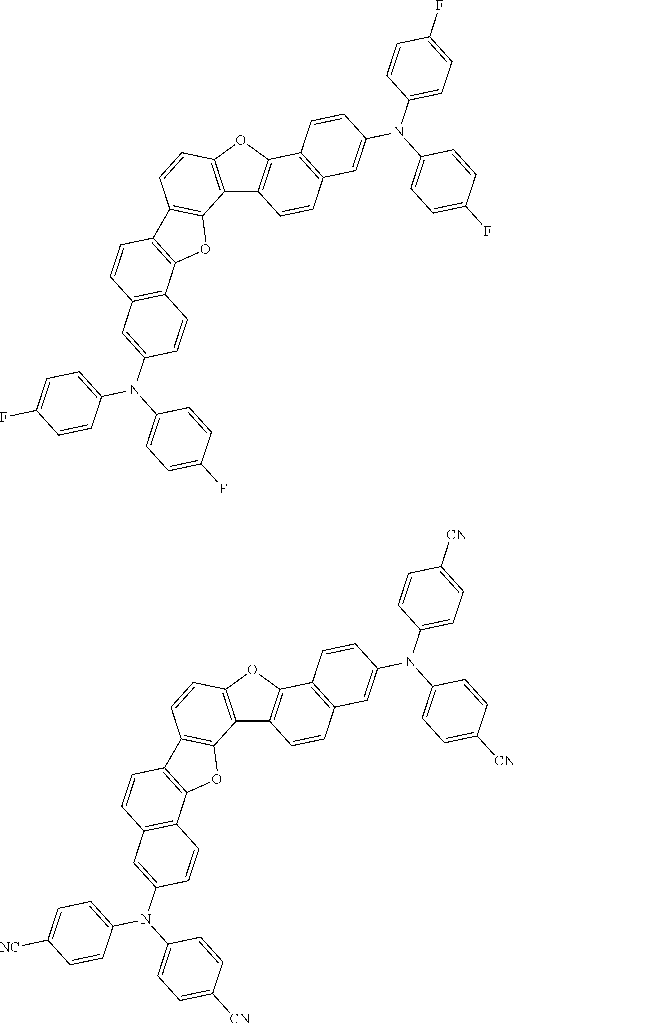 [SMILES: Fc1ccc(N(c2ccc(F)cc2)c2ccc3c(ccc4c5ccc6oc7c8ccc(N(c9ccc(F)cc9)c9ccc(F)cc9)cc8ccc7c6c5oc34)c2)cc1 ; N#Cc1ccc(N(c2ccc(C#N)cc2)c2ccc3c(ccc4c5ccc6oc7c8ccc(N(c9ccc(C#N)cc9)c9ccc(C#N)cc9)cc8ccc7c6c5oc34)c2)cc1]

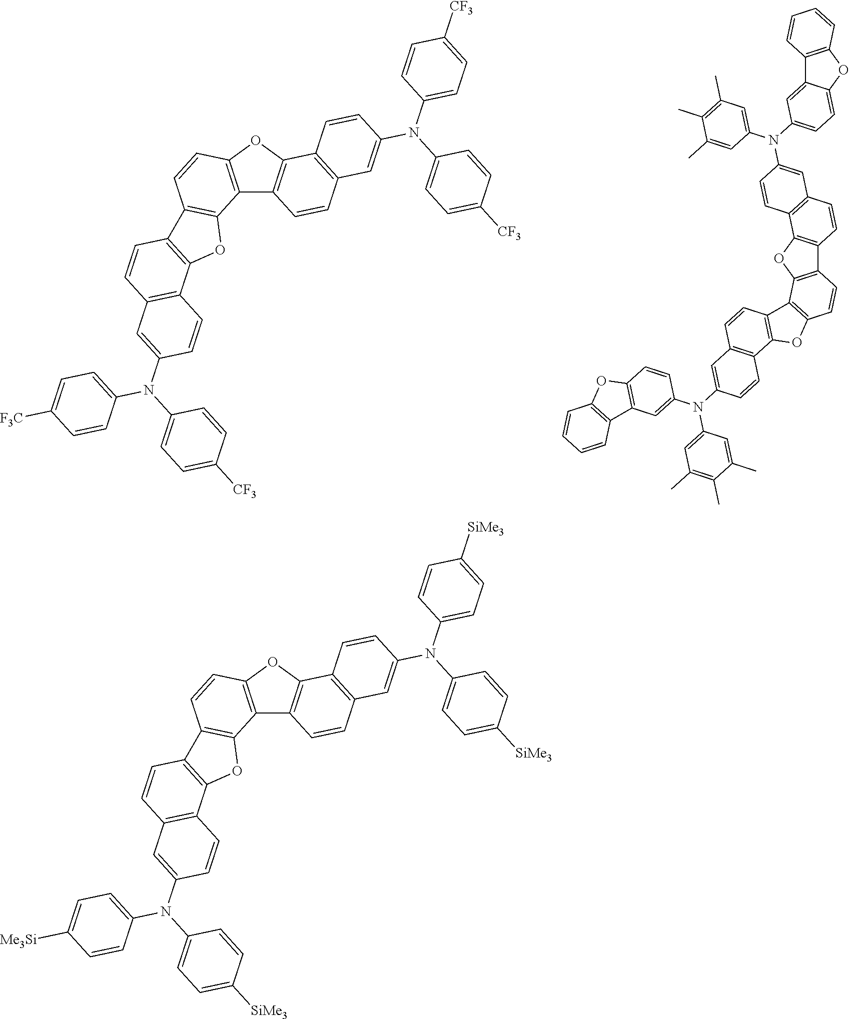 [SMILES: FC(F)(F)c1ccc(N(c2ccc(C(F)(F)F)cc2)c2ccc3c(ccc4c5ccc6oc7c8ccc(N(c9ccc(C(F)(F)F)cc9)c9ccc(C(F)(F)F)cc9)cc8ccc7c6c5oc34)c2)cc1 ; Cc1cc(N(c2ccc3c(ccc4c5ccc6oc7c8ccc(N(c9cc(C)c(C)c(C)c9)c9ccc%10oc%11ccccc%11c%10c9)cc8ccc7c6c5oc34)c2)c2ccc3oc4ccccc4c3c2)cc(C)c1C ; C[Si](C)(C)c1ccc(N(c2ccc([Si](C)(C)C)cc2)c2ccc3c(ccc4c5ccc6oc7c8ccc(N(c9ccc([Si](C)(C)C)cc9)c9ccc([Si](C)(C)C)cc9)cc8ccc7c6c5oc34)c2)cc1]

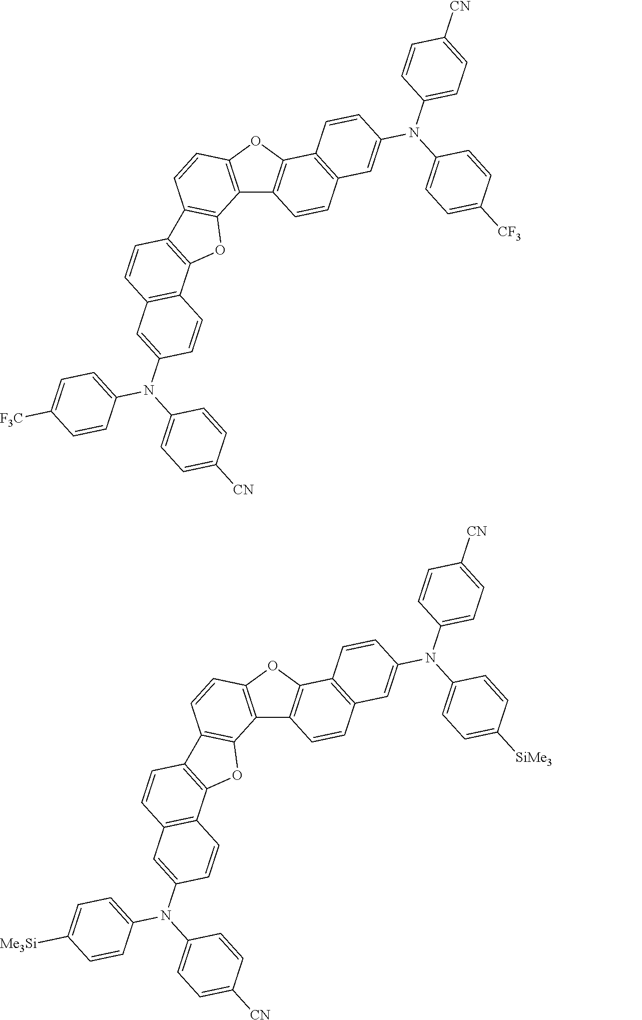 [SMILES: N#Cc1ccc(N(c2ccc(C(F)(F)F)cc2)c2ccc3c(ccc4c5ccc6oc7c8ccc(N(c9ccc(C#N)cc9)c9ccc(C(F)(F)F)cc9)cc8ccc7c6c5oc34)c2)cc1 ; C[Si](C)(C)c1ccc(N(c2ccc(C#N)cc2)c2ccc3c(ccc4c5ccc6oc7c8ccc(N(c9ccc(C#N)cc9)c9ccc([Si](C)(C)C)cc9)cc8ccc7c6c5oc34)c2)cc1]

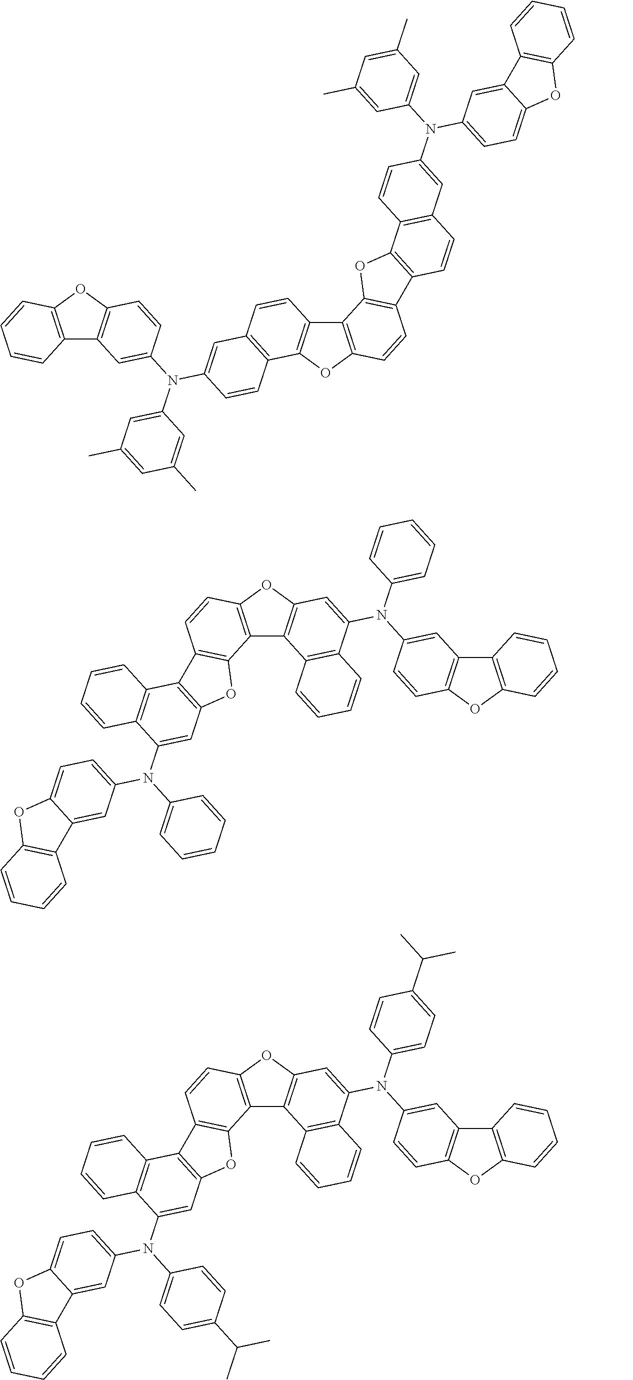 [SMILES: Cc1cc(C)cc(N(c2ccc3c(ccc4c5ccc6oc7c8ccc(N(c9cc(C)cc(C)c9)c9ccc%10oc%11ccccc%11c%10c9)cc8ccc7c6c5oc34)c2)c2ccc3oc4ccccc4c3c2)c1 ; c1ccc(N(c2ccc3oc4ccccc4c3c2)c2cc3oc4c(ccc5oc6cc(N(c7ccccc7)c7ccc8oc9ccccc9c8c7)c7ccccc7c6c54)c3c3ccccc23)cc1 ; CC(C)c1ccc(N(c2ccc3oc4ccccc4c3c2)c2cc3oc4c(ccc5oc6cc(N(c7ccc(C(C)C)cc7)c7ccc8oc9ccccc9c8c7)c7ccccc7c6c54)c3c3ccccc23)cc1]

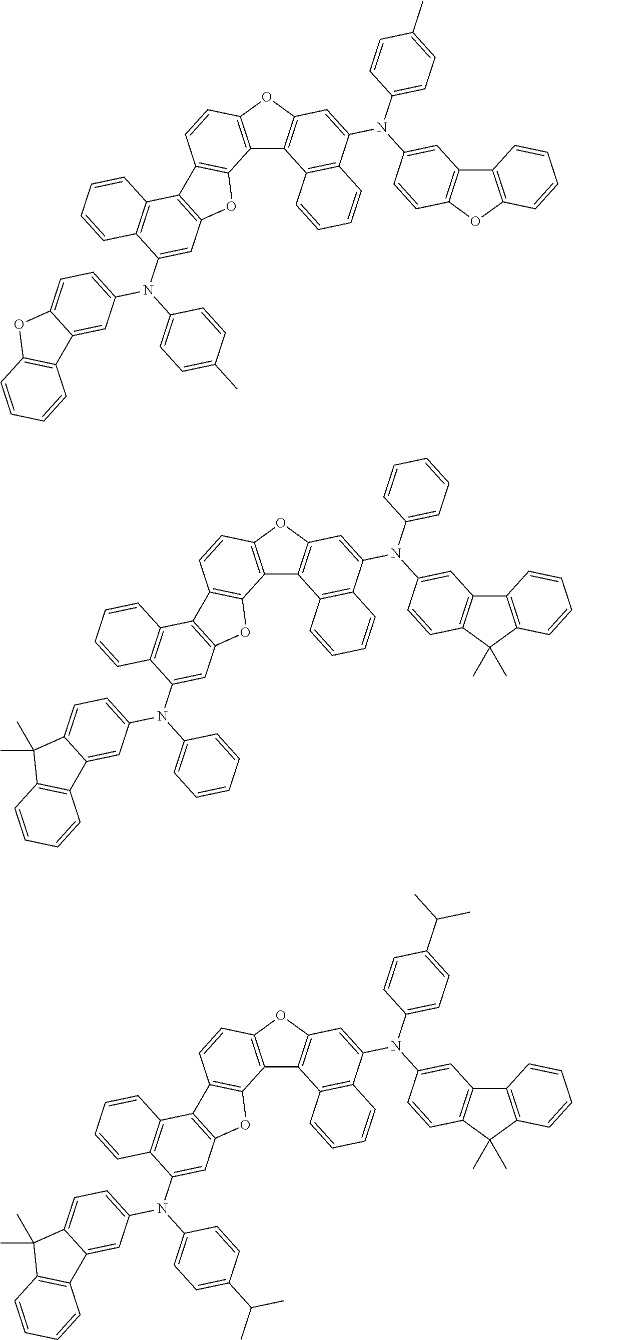 [SMILES: Cc1ccc(N(c2ccc3oc4ccccc4c3c2)c2cc3oc4c(ccc5oc6cc(N(c7ccc(C)cc7)c7ccc8oc9ccccc9c8c7)c7ccccc7c6c54)c3c3ccccc23)cc1 ; CC1(C)c2ccccc2-c2cc(N(c3ccccc3)c3cc4oc5c(ccc6oc7cc(N(c8ccccc8)c8ccc9c(c8)-c8ccccc8C9(C)C)c8ccccc8c7c65)c4c4ccccc34)ccc21 ; CC(C)c1ccc(N(c2ccc3c(c2)-c2ccccc2C3(C)C)c2cc3oc4c(ccc5oc6cc(N(c7ccc(C(C)C)cc7)c7ccc8c(c7)-c7ccccc7C8(C)C)c7ccccc7c6c54)c3c3ccccc23)cc1]

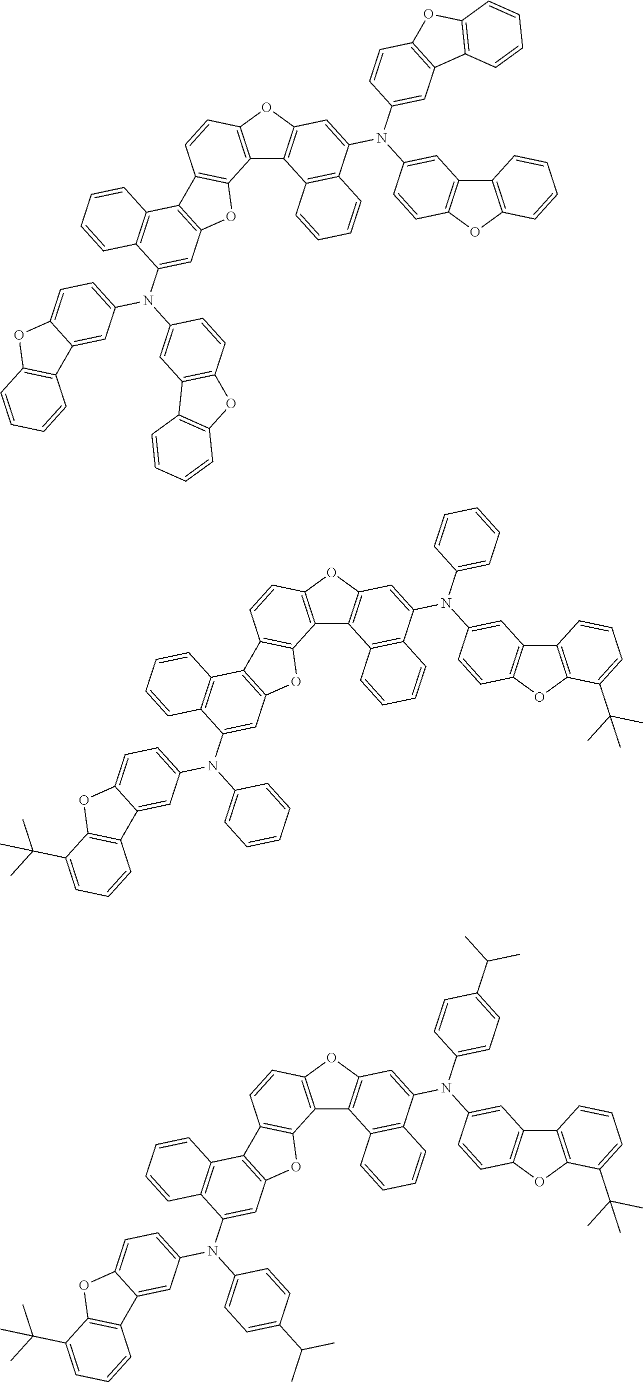

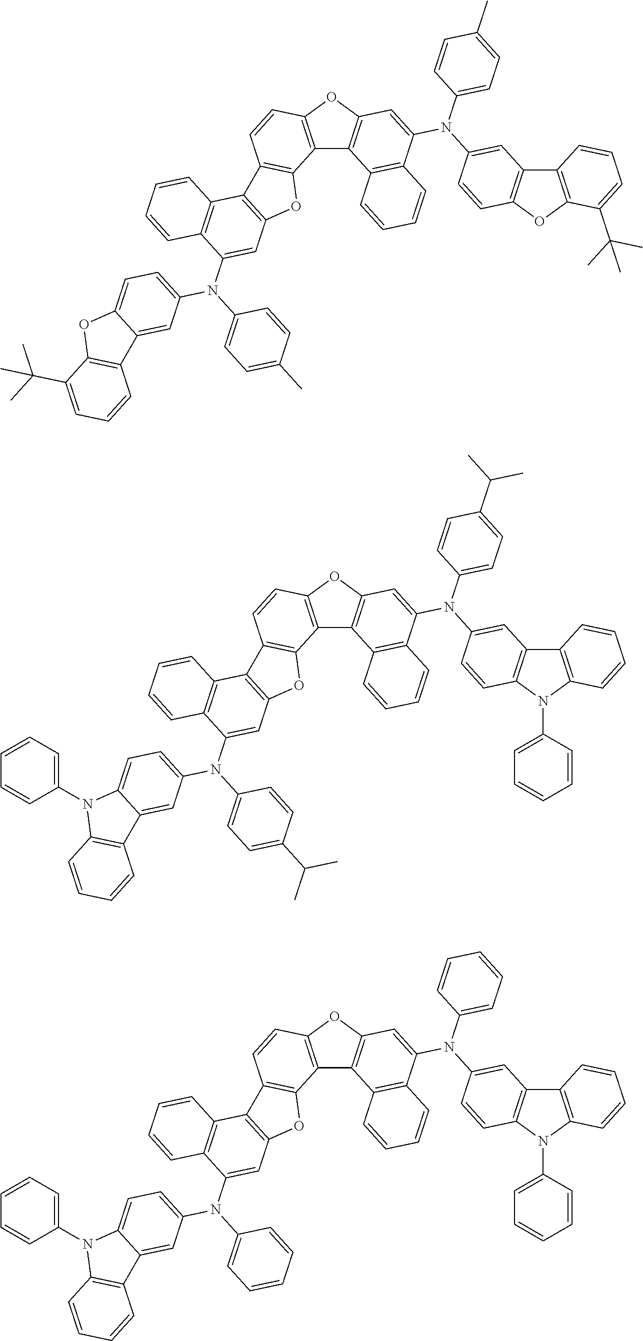 [SMILES: Cc1ccc(N(c2ccc3oc4c(C(C)(C)C)cccc4c3c2)c2cc3oc4c(ccc5oc6cc(N(c7ccc(C)cc7)c7ccc8oc9c(C(C)(C)C)cccc9c8c7)c7ccccc7c6c54)c3c3ccccc23)cc1 ; CC(C)c1ccc(N(c2ccc3c(c2)c2ccccc2n3-c2ccccc2)c2cc3oc4c(ccc5oc6cc(N(c7ccc(C(C)C)cc7)c7ccc8c(c7)c7ccccc7n8-c7ccccc7)c7ccccc7c6c54)c3c3ccccc23)cc1 ; c1ccc(N(c2ccc3c(c2)c2ccccc2n3-c2ccccc2)c2cc3oc4c(ccc5oc6cc(N(c7ccccc7)c7ccc8c(c7)c7ccccc7n8-c7ccccc7)c7ccccc7c6c54)c3c3ccccc23)cc1]

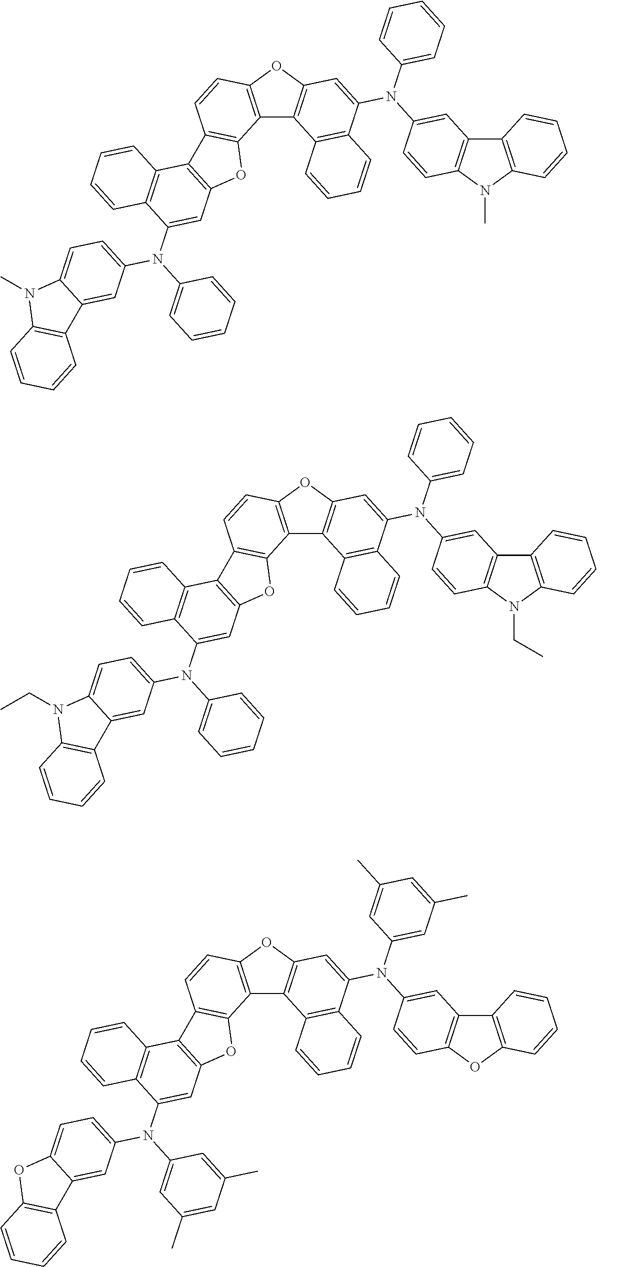 [SMILES: Cn1c2ccccc2c2cc(N(c3ccccc3)c3cc4oc5c(ccc6oc7cc(N(c8ccccc8)c8ccc9c(c8)c8ccccc8n9C)c8ccccc8c7c65)c4c4ccccc34)ccc21 ; CCn1c2ccccc2c2cc(N(c3ccccc3)c3cc4oc5c(ccc6oc7cc(N(c8ccccc8)c8ccc9c(c8)c8ccccc8n9CC)c8ccccc8c7c65)c4c4ccccc34)ccc21 ; Cc1cc(C)cc(N(c2ccc3oc4ccccc4c3c2)c2cc3oc4c(ccc5oc6cc(N(c7cc(C)cc(C)c7)c7ccc8oc9ccccc9c8c7)c7ccccc7c6c54)c3c3ccccc23)c1]

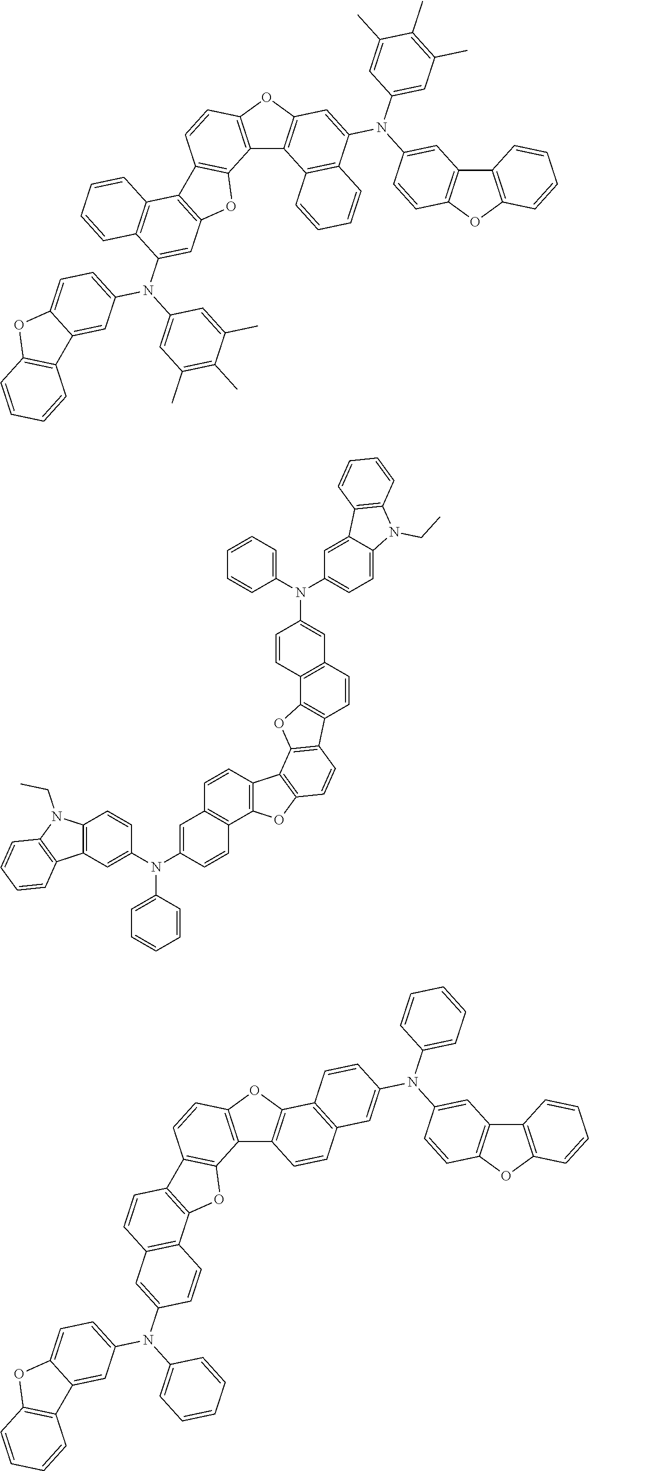 [SMILES: Cc1cc(N(c2ccc3oc4ccccc4c3c2)c2cc3oc4c(ccc5oc6cc(N(c7cc(C)c(C)c(C)c7)c7ccc8oc9ccccc9c8c7)c7ccccc7c6c54)c3c3ccccc23)cc(C)c1C ; CCn1c2ccccc2c2cc(N(c3ccccc3)c3ccc4c(ccc5c6ccc7oc8c9ccc(N(c%10ccccc%10)c%10ccc%11c(c%10)c%10ccccc%10n%11CC)cc9ccc8c7c6oc45)c3)ccc21 ; c1ccc(N(c2ccc3c(ccc4c5ccc6oc7c8ccc(N(c9ccccc9)c9ccc%10oc%11ccccc%11c%10c9)cc8ccc7c6c5oc34)c2)c2ccc3oc4ccccc4c3c2)cc1]

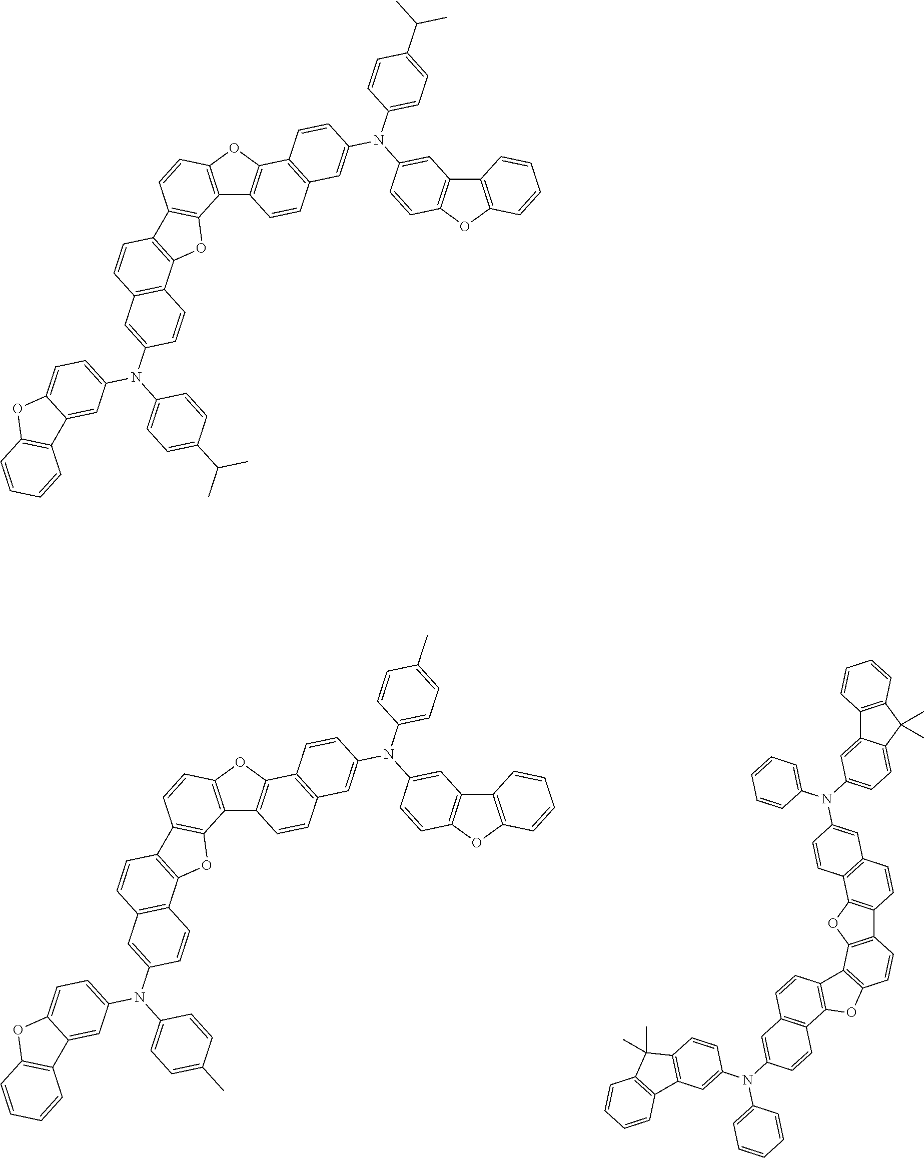 [SMILES: CC(C)c1ccc(N(c2ccc3c(ccc4c5ccc6oc7c8ccc(N(c9ccc(C(C)C)cc9)c9ccc%10oc%11ccccc%11c%10c9)cc8ccc7c6c5oc34)c2)c2ccc3oc4ccccc4c3c2)cc1 ; Cc1ccc(N(c2ccc3c(ccc4c5ccc6oc7c8ccc(N(c9ccc(C)cc9)c9ccc%10oc%11ccccc%11c%10c9)cc8ccc7c6c5oc34)c2)c2ccc3oc4ccccc4c3c2)cc1 ; CC1(C)c2ccccc2-c2cc(N(c3ccccc3)c3ccc4c(ccc5c6ccc7oc8c9ccc(N(c%10ccccc%10)c%10ccc%11c(c%10)-c%10ccccc%10C%11(C)C)cc9ccc8c7c6oc45)c3)ccc21]

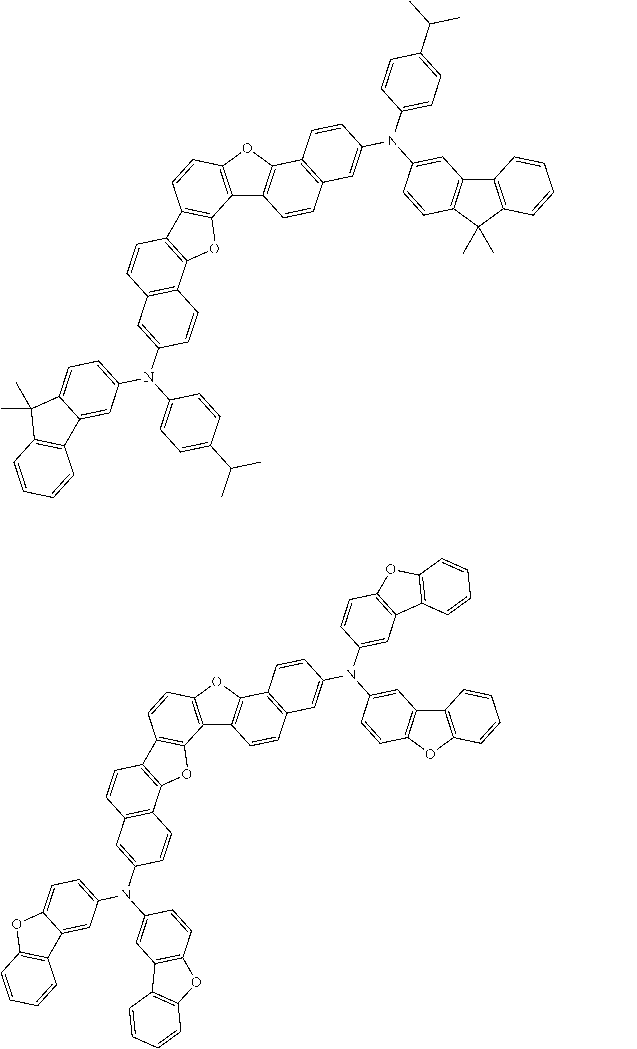 [SMILES: CC(C)c1ccc(N(c2ccc3c(c2)-c2ccccc2C3(C)C)c2ccc3c(ccc4c5ccc6oc7c8ccc(N(c9ccc(C(C)C)cc9)c9ccc%10c(c9)-c9ccccc9C%10(C)C)cc8ccc7c6c5oc34)c2)cc1 ; c1ccc2c(c1)oc1ccc(N(c3ccc4c(ccc5c6ccc7oc8c9ccc(N(c%10ccc%11oc%12ccccc%12c%11c%10)c%10ccc%11oc%12ccccc%12c%11c%10)cc9ccc8c7c6oc45)c3)c3ccc4oc5ccccc5c4c3)cc12]

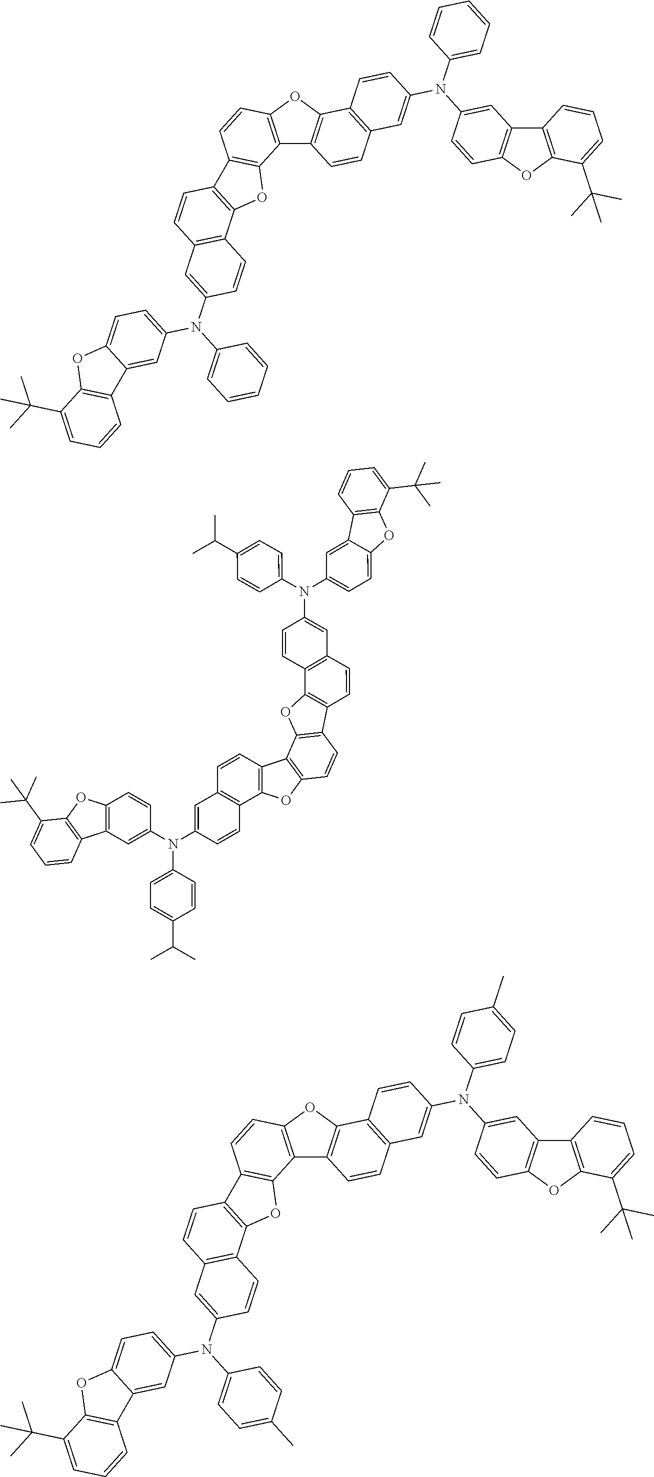

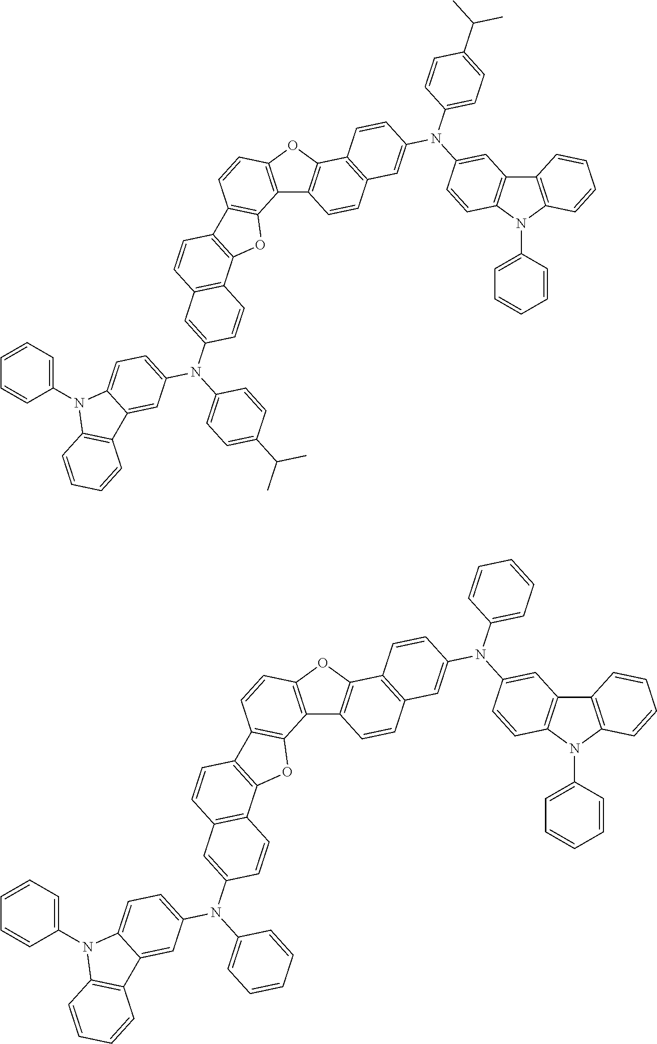 [SMILES: CC(C)c1ccc(N(c2ccc3c(ccc4c5ccc6oc7c8ccc(N(c9ccc(C(C)C)cc9)c9ccc%10c(c9)c9ccccc9n%10-c9ccccc9)cc8ccc7c6c5oc34)c2)c2ccc3c(c2)c2ccccc2n3-c2ccccc2)cc1 ; c1ccc(N(c2ccc3c(ccc4c5ccc6oc7c8ccc(N(c9ccccc9)c9ccc%10c(c9)c9ccccc9n%10-c9ccccc9)cc8ccc7c6c5oc34)c2)c2ccc3c(c2)c2ccccc2n3-c2ccccc2)cc1]

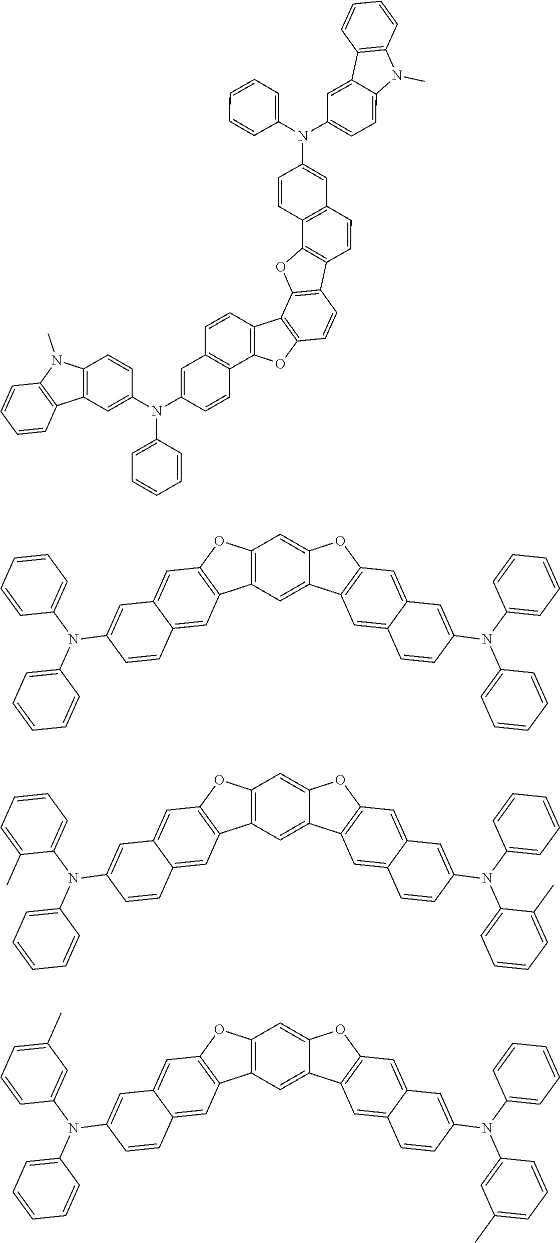 [SMILES: Cn1c2ccccc2c2cc(N(c3ccccc3)c3ccc4c(ccc5c6ccc7oc8c9ccc(N(c%10ccccc%10)c%10ccc%11c(c%10)c%10ccccc%10n%11C)cc9ccc8c7c6oc45)c3)ccc21 ; c1ccc(N(c2ccccc2)c2ccc3cc4c(cc3c2)oc2cc3oc5cc6cc(N(c7ccccc7)c7ccccc7)ccc6cc5c3cc24)cc1 ; Cc1ccccc1N(c1ccccc1)c1ccc2cc3c(cc2c1)oc1cc2oc4cc5cc(N(c6ccccc6)c6ccccc6C)ccc5cc4c2cc13 ; Cc1cccc(N(c2ccccc2)c2ccc3cc4c(cc3c2)oc2cc3oc5cc6cc(N(c7ccccc7)c7cccc(C)c7)ccc6cc5c3cc24)c1]

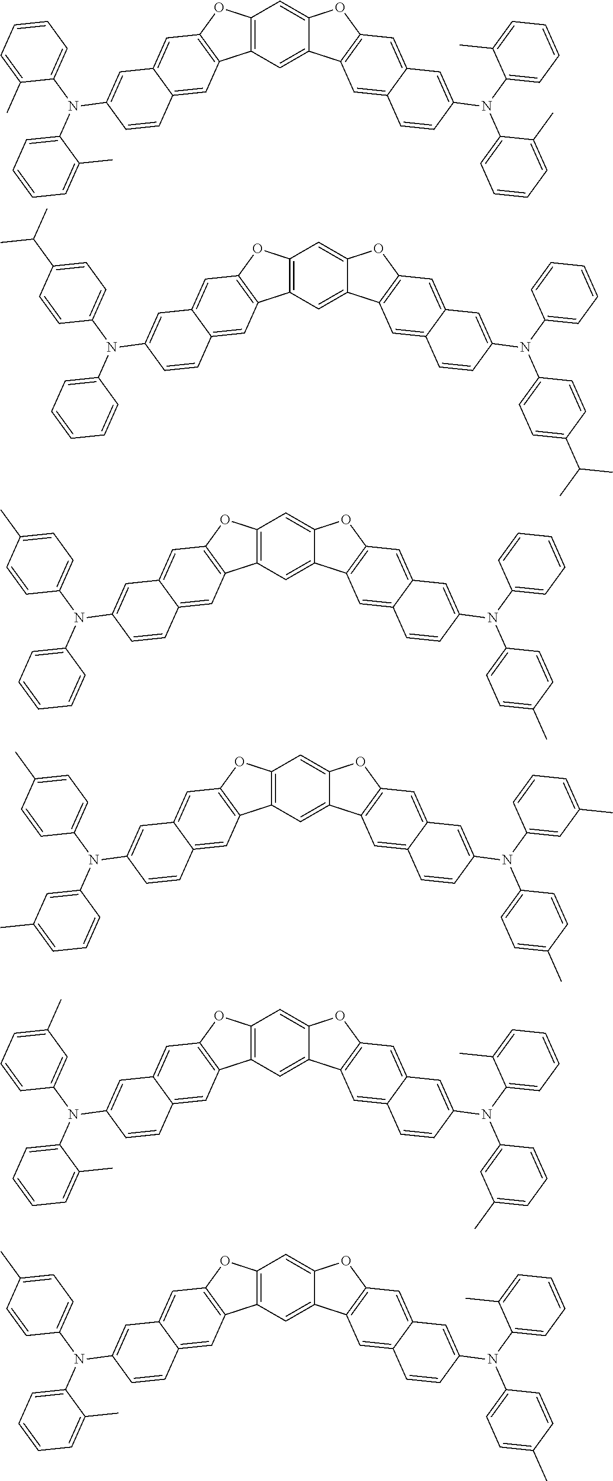

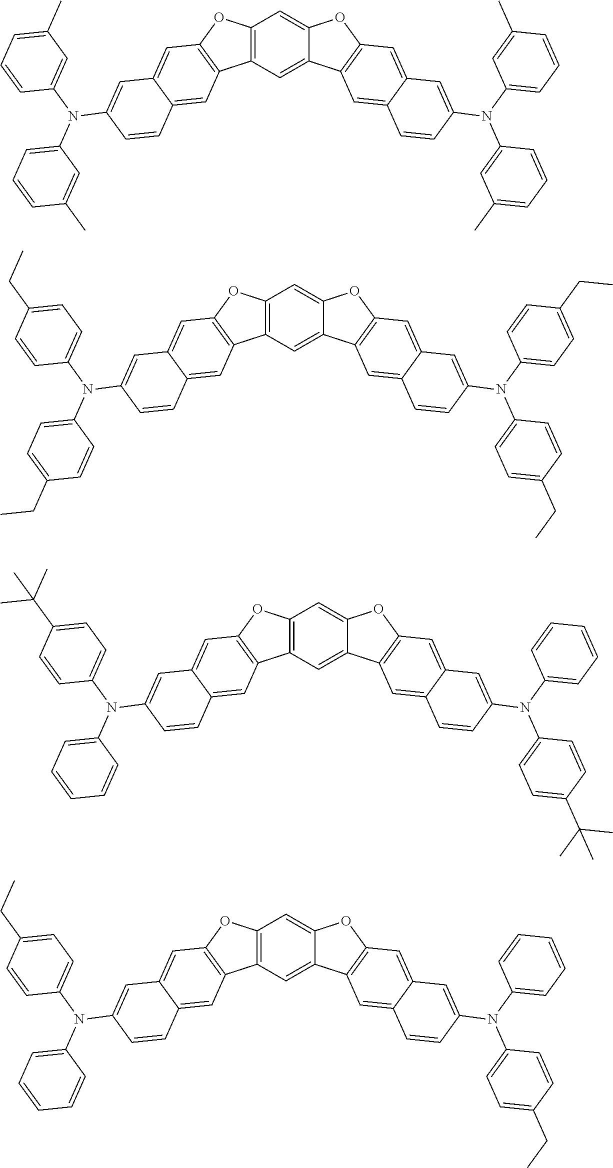

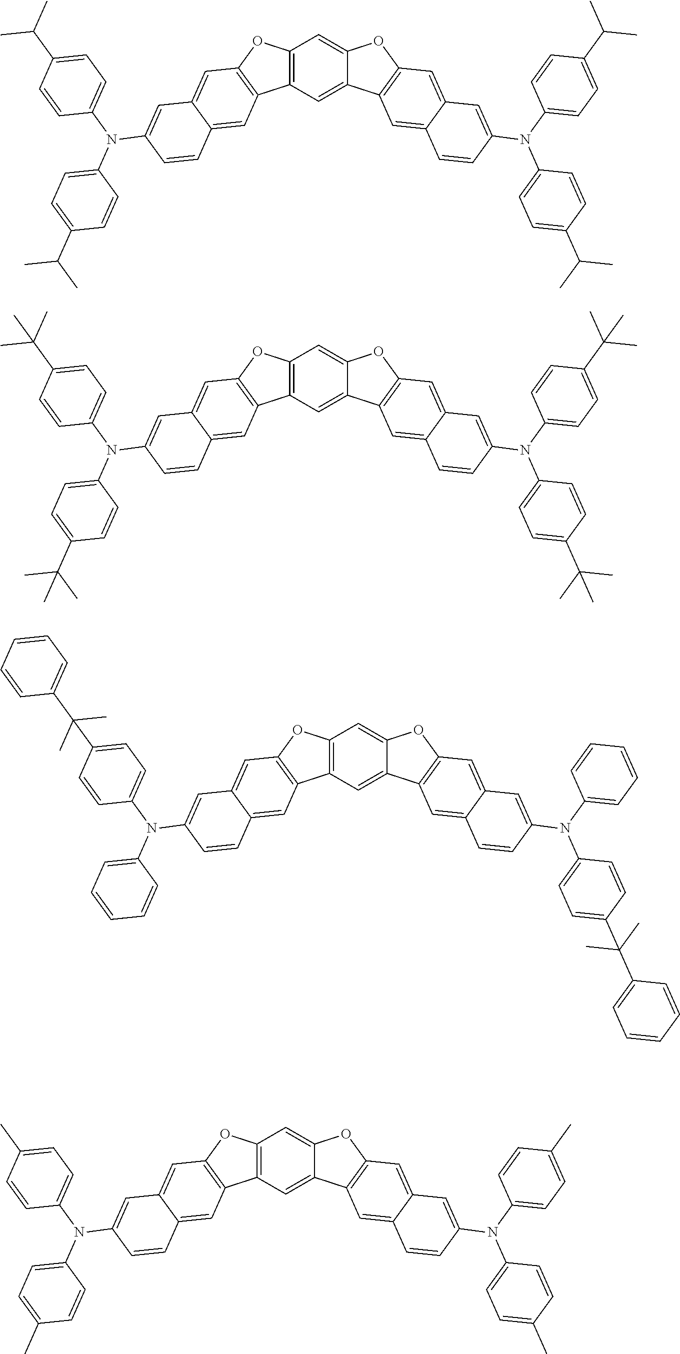

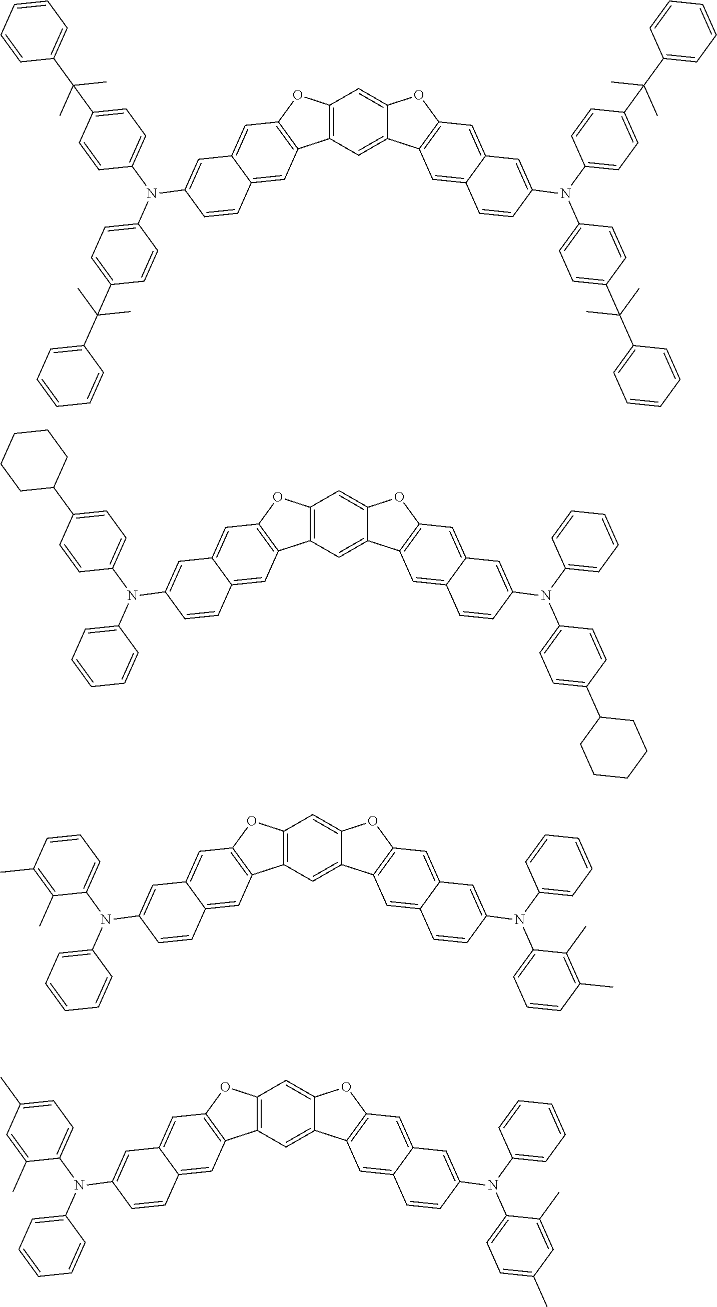

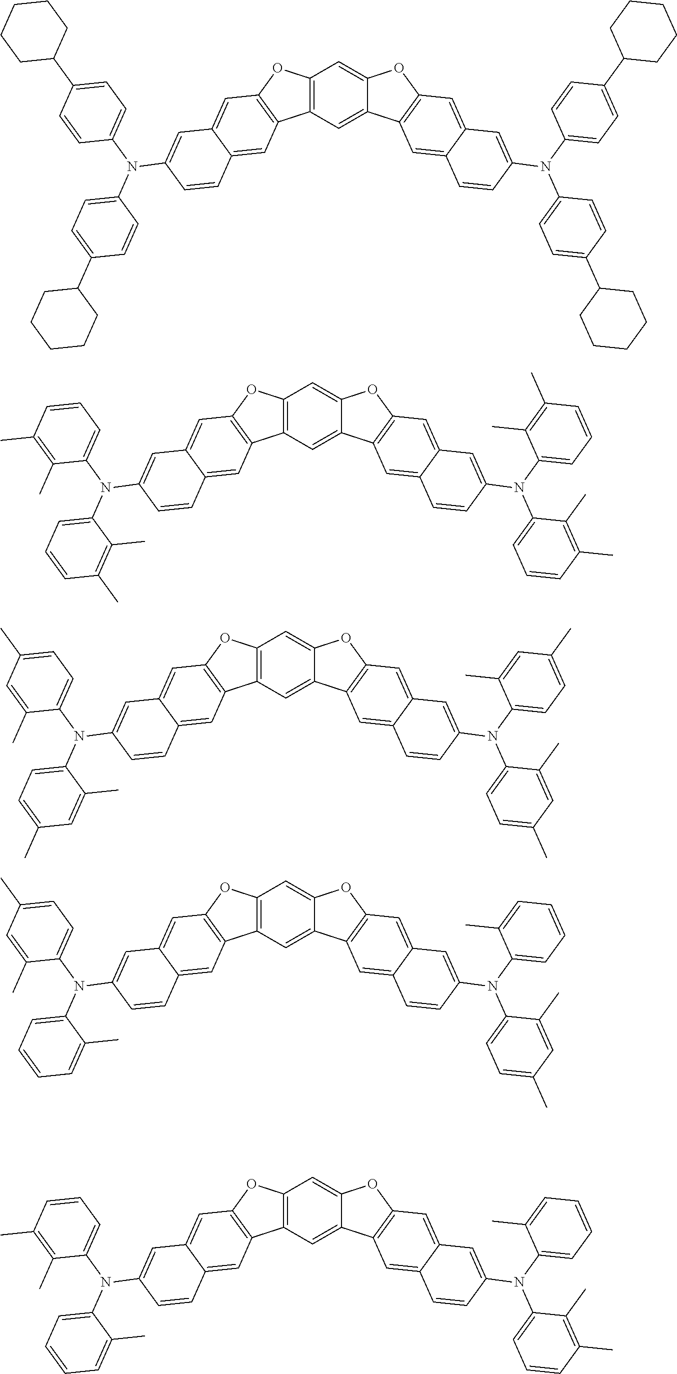

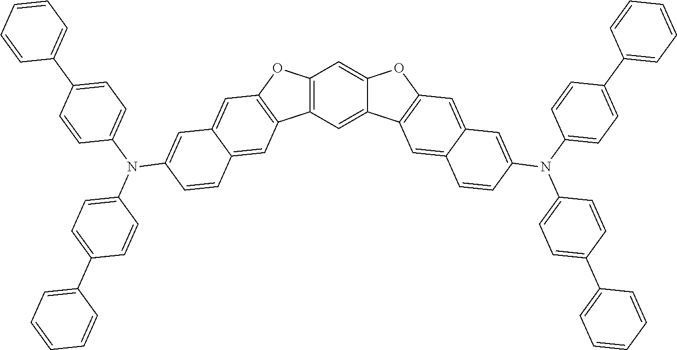 [SMILES: c1ccc(-c2ccc(N(c3ccc(-c4ccccc4)cc3)c3ccc4cc5c(cc4c3)oc3cc4oc6cc7cc(N(c8ccc(-c9ccccc9)cc8)c8ccc(-c9ccccc9)cc8)ccc7cc6c4cc35)cc2)cc1]

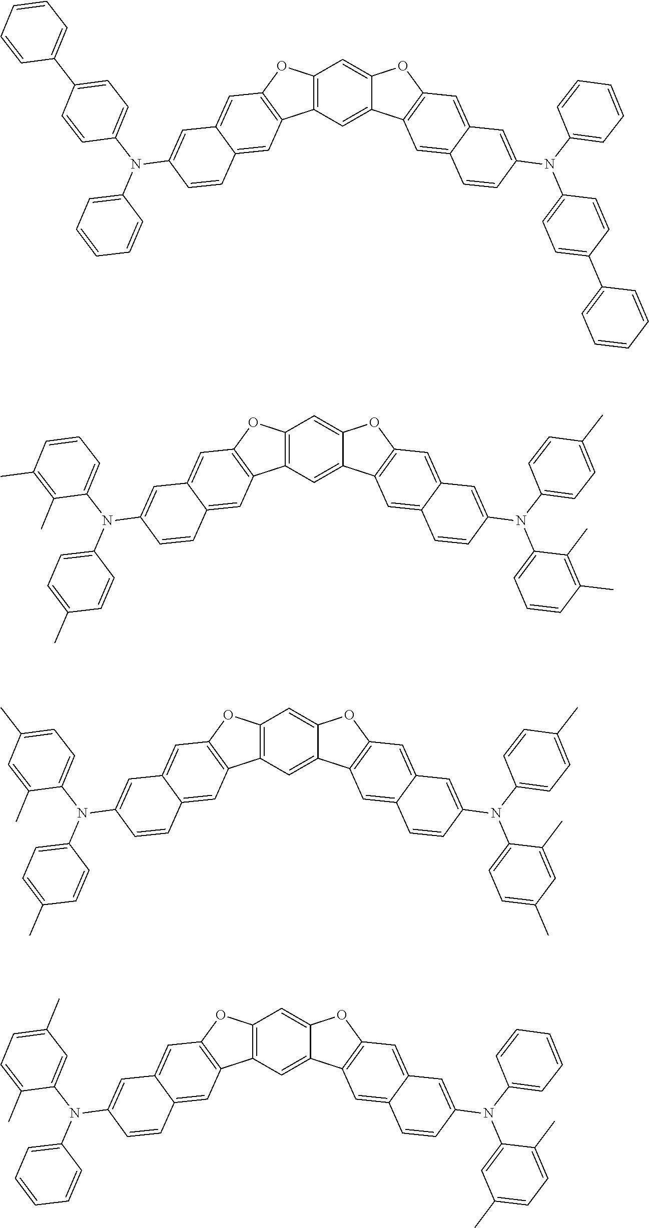

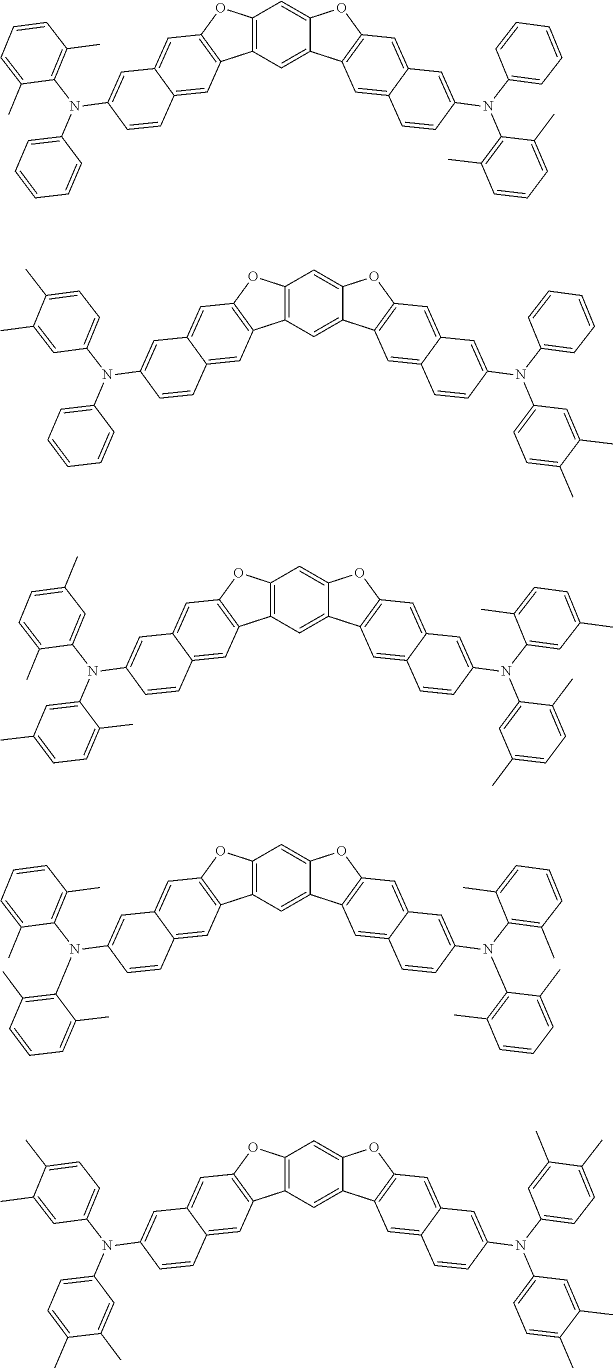

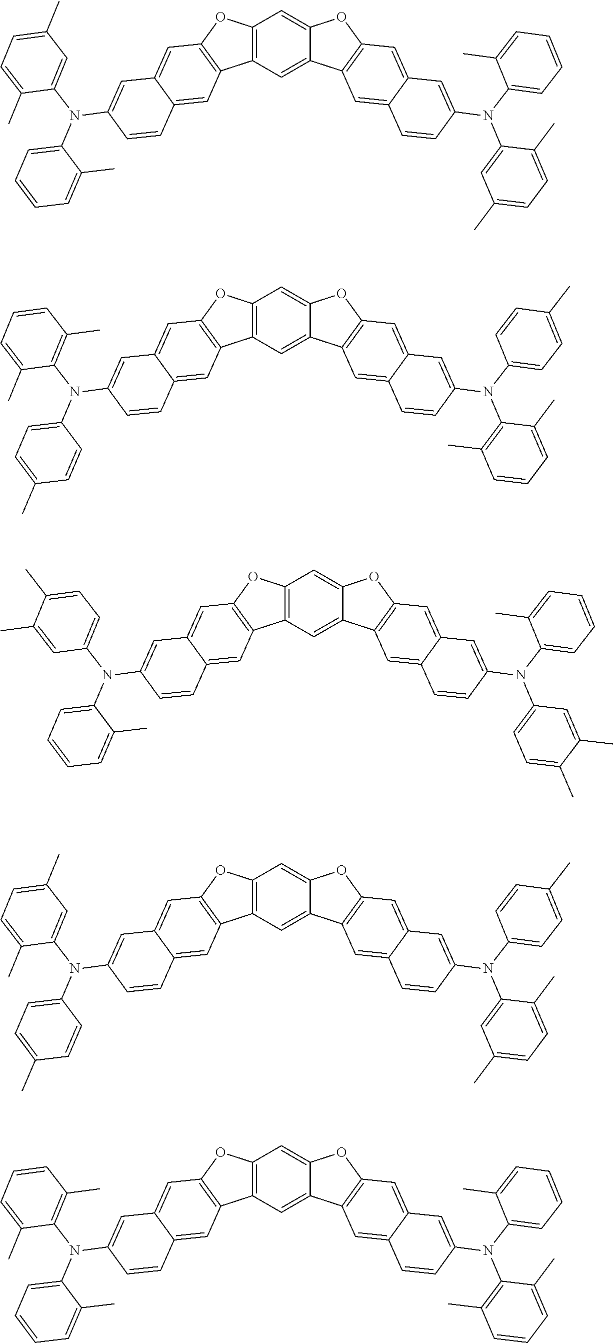

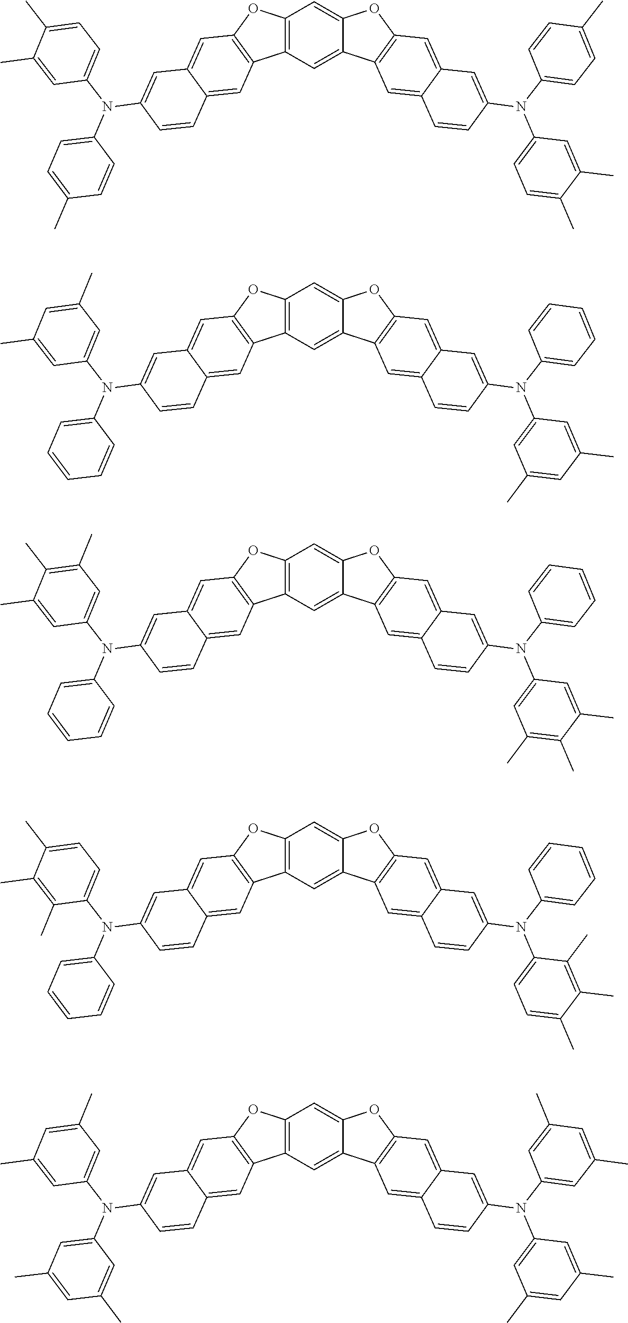

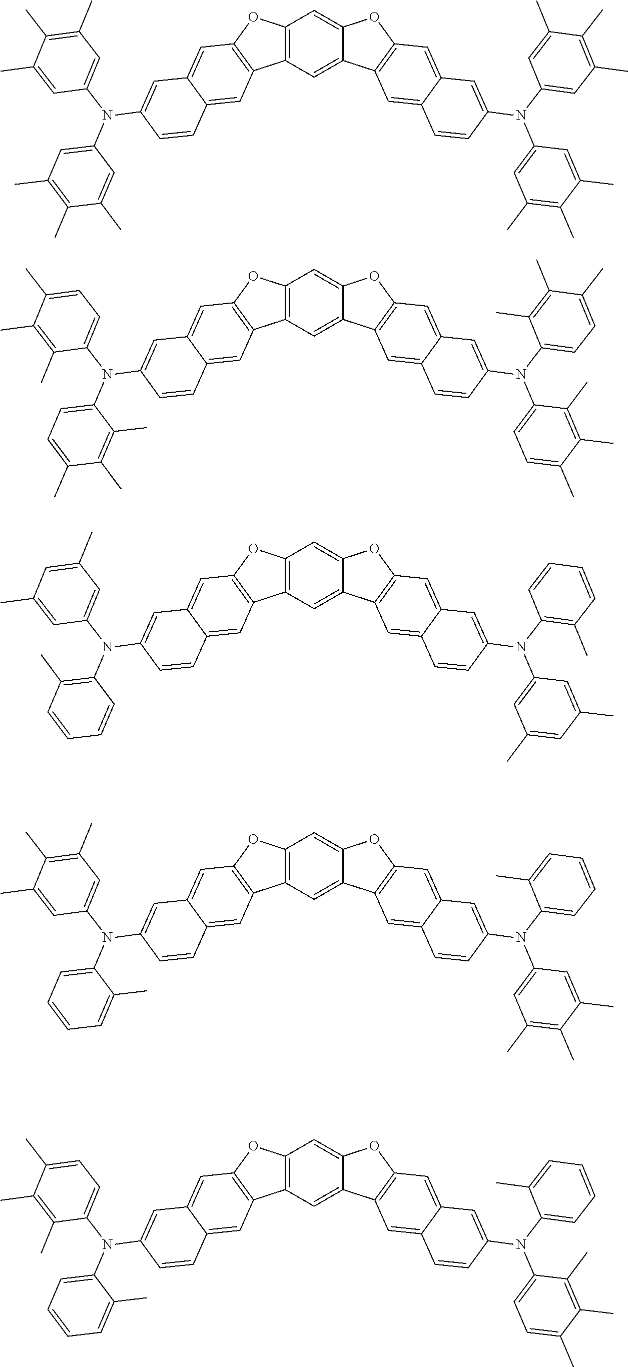

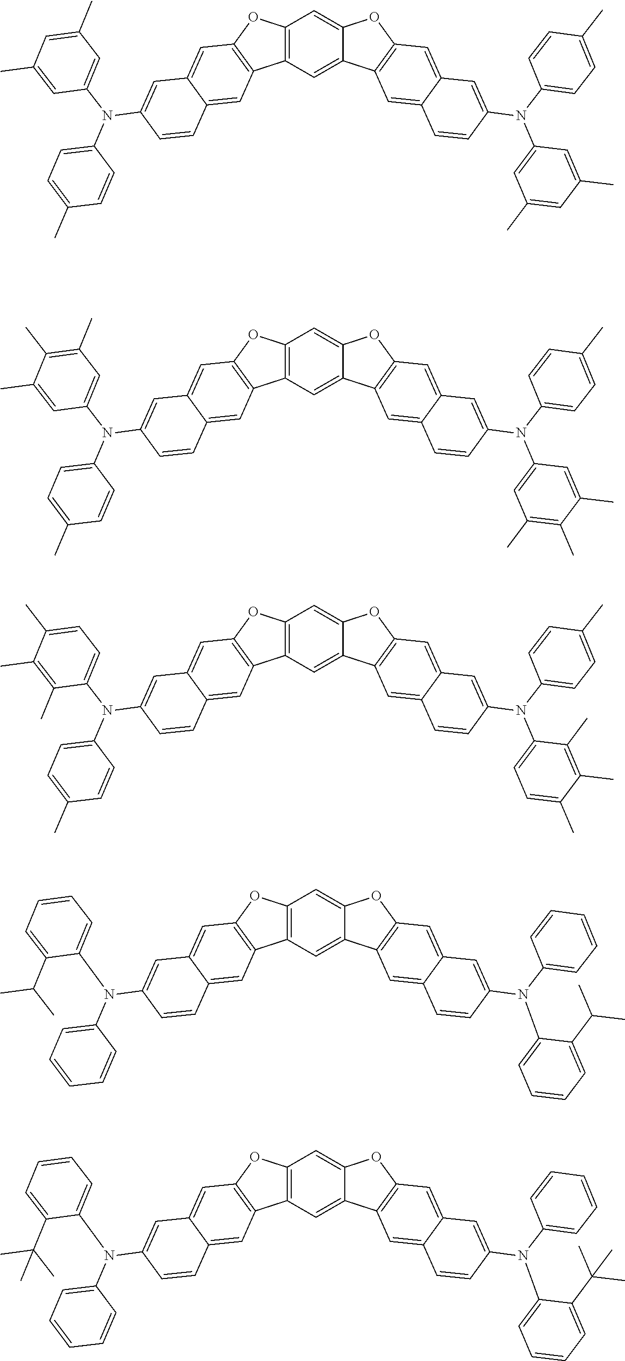

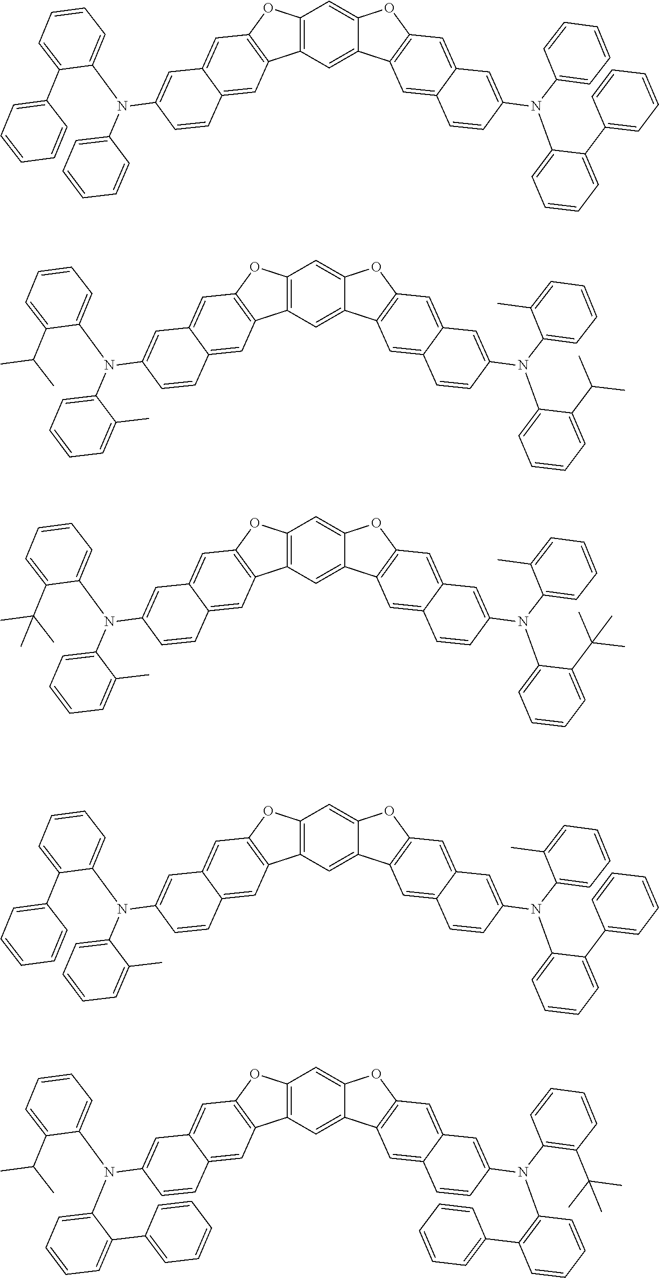

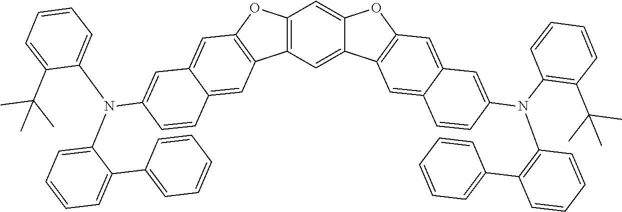 [SMILES: CC(C)(C)c1ccccc1N(c1ccc2cc3c(cc2c1)oc1cc2oc4cc5cc(N(c6ccccc6-c6ccccc6)c6ccccc6C(C)(C)C)ccc5cc4c2cc13)c1ccccc1-c1ccccc1]

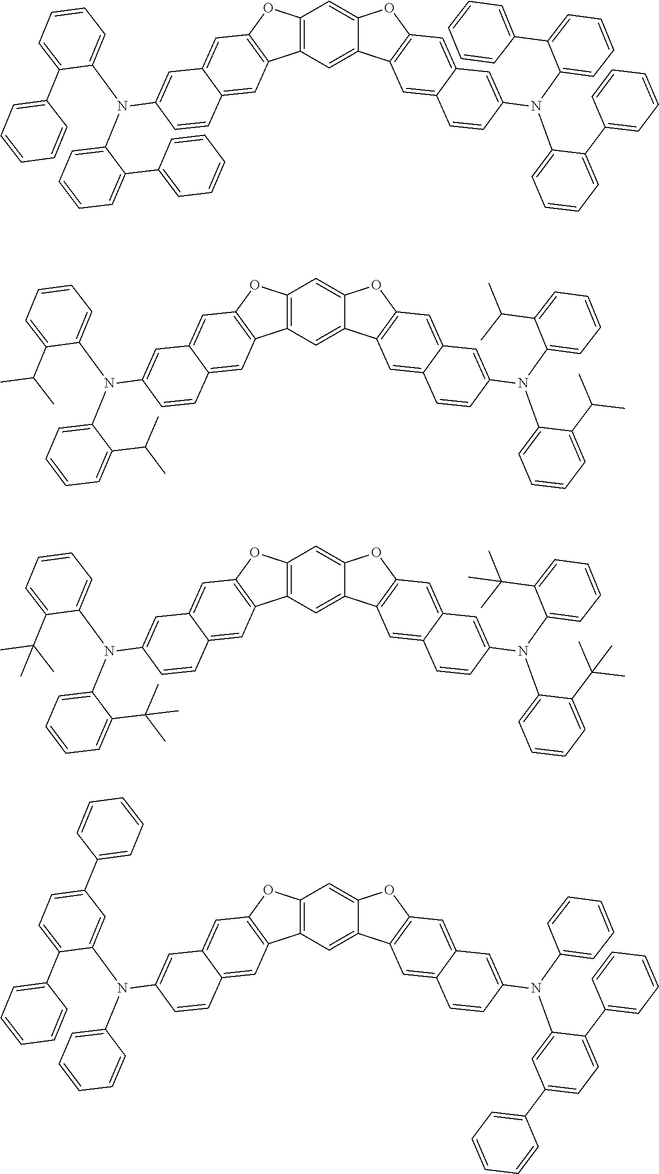

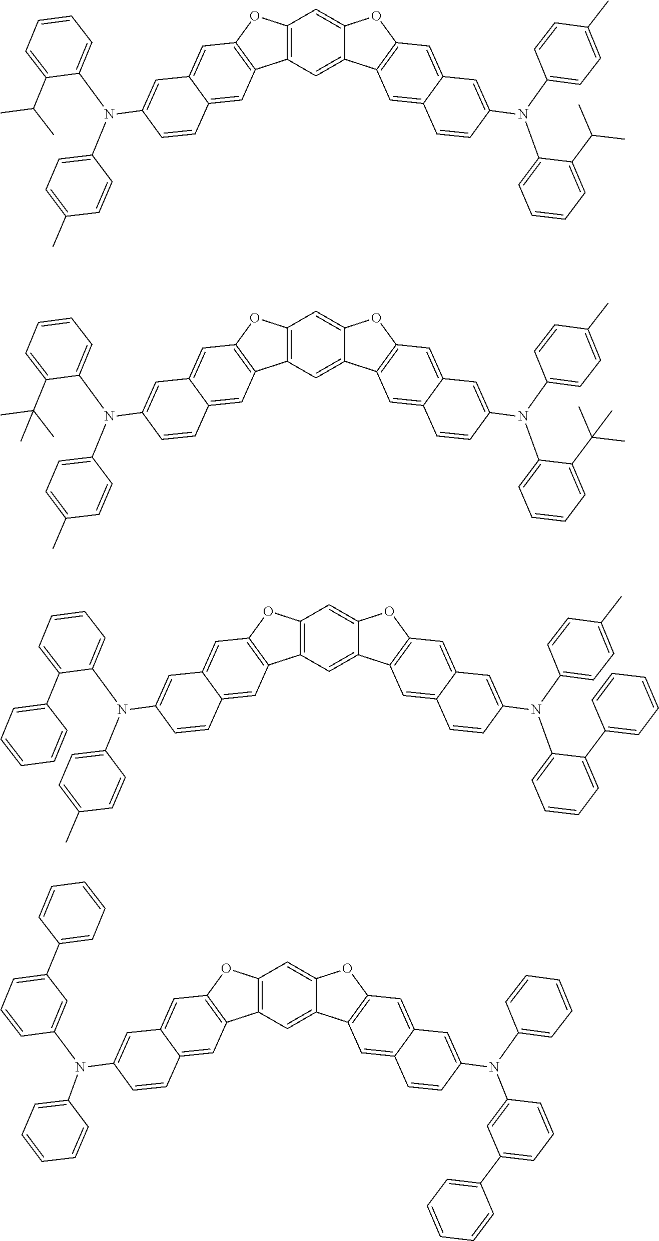

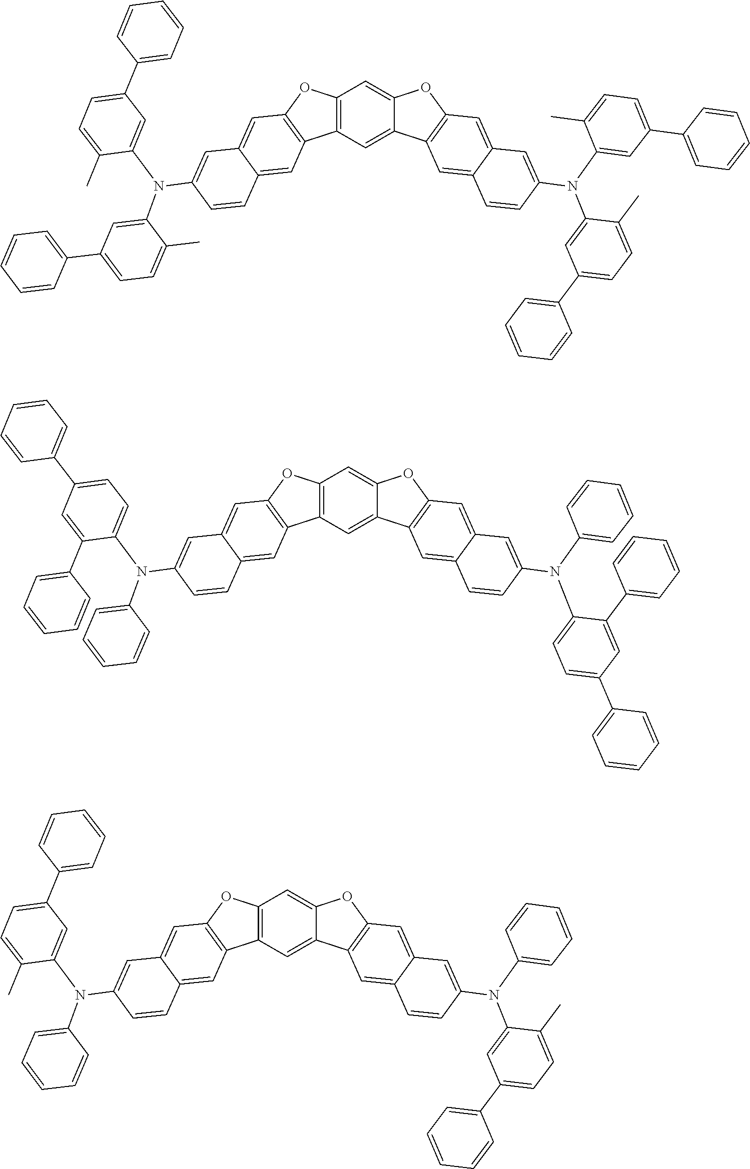 [SMILES: Cc1ccc(-c2ccccc2)cc1N(c1ccc2cc3c(cc2c1)oc1cc2oc4cc5cc(N(c6cc(-c7ccccc7)ccc6C)c6cc(-c7ccccc7)ccc6C)ccc5cc4c2cc13)c1cc(-c2ccccc2)ccc1C ; c1ccc(-c2ccc(N(c3ccccc3)c3ccc4cc5c(cc4c3)oc3cc4oc6cc7cc(N(c8ccccc8)c8ccc(-c9ccccc9)cc8-c8ccccc8)ccc7cc6c4cc35)c(-c3ccccc3)c2)cc1 ; Cc1ccc(-c2ccccc2)cc1N(c1ccccc1)c1ccc2cc3c(cc2c1)oc1cc2oc4cc5cc(N(c6ccccc6)c6cc(-c7ccccc7)ccc6C)ccc5cc4c2cc13]

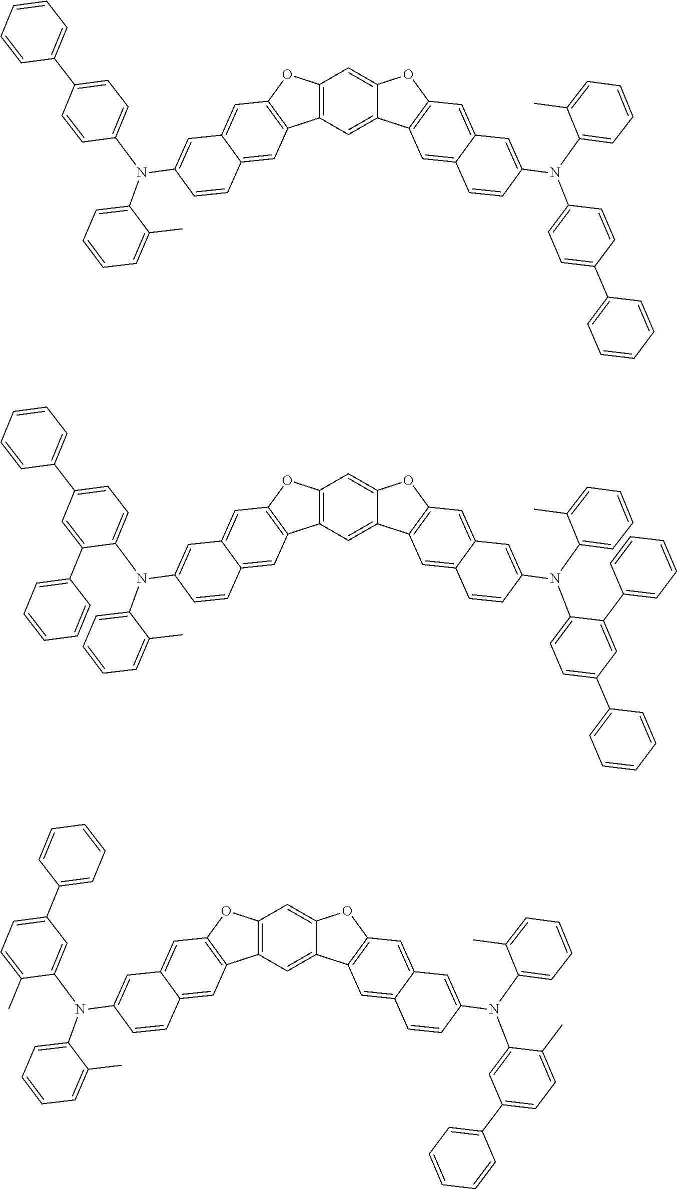 [SMILES: Cc1ccccc1N(c1ccc(-c2ccccc2)cc1)c1ccc2cc3c(cc2c1)oc1cc2oc4cc5cc(N(c6ccc(-c7ccccc7)cc6)c6ccccc6C)ccc5cc4c2cc13 ; Cc1ccccc1N(c1ccc2cc3c(cc2c1)oc1cc2oc4cc5cc(N(c6ccccc6C)c6ccc(-c7ccccc7)cc6-c6ccccc6)ccc5cc4c2cc13)c1ccc(-c2ccccc2)cc1-c1ccccc1 ; Cc1ccccc1N(c1ccc2cc3c(cc2c1)oc1cc2oc4cc5cc(N(c6ccccc6C)c6cc(-c7ccccc7)ccc6C)ccc5cc4c2cc13)c1cc(-c2ccccc2)ccc1C]

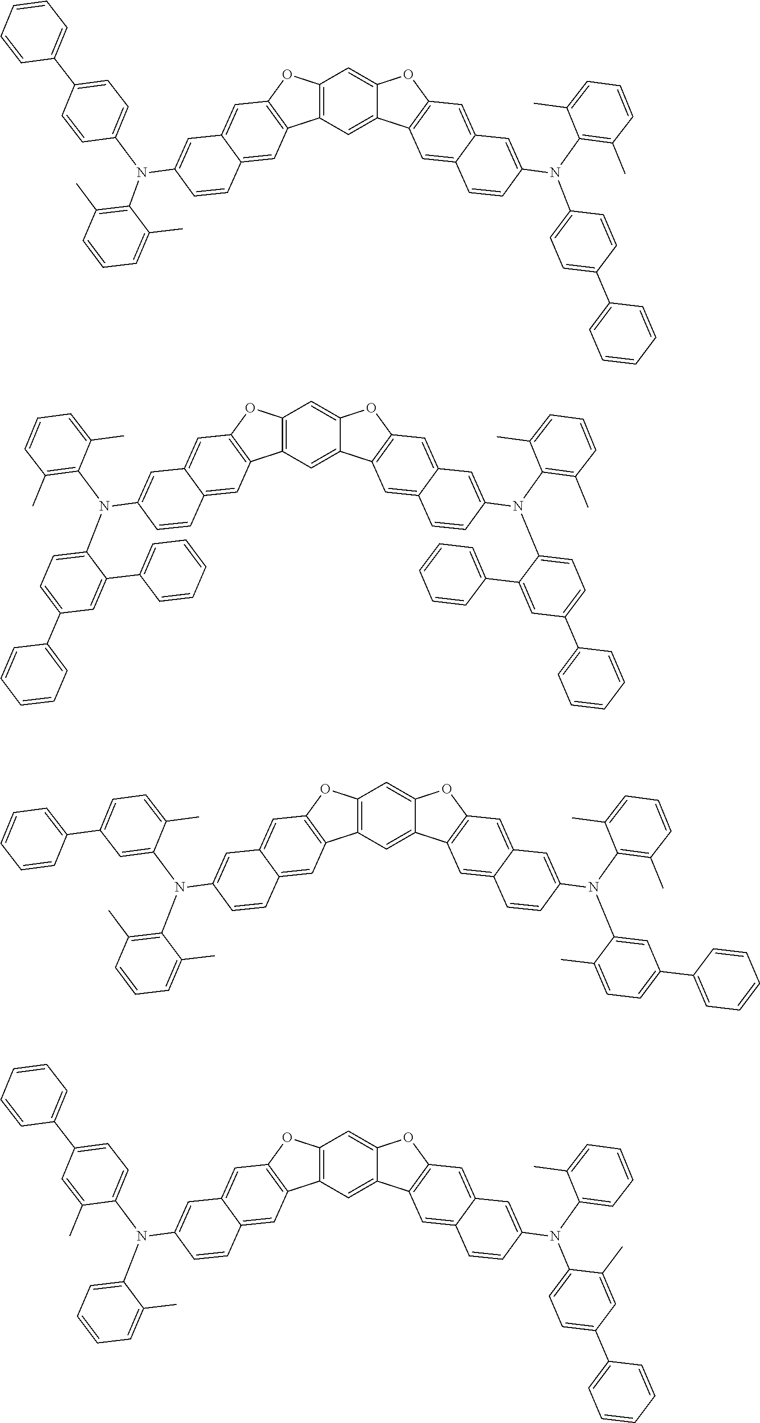 [SMILES: Cc1cccc(C)c1N(c1ccc(-c2ccccc2)cc1)c1ccc2cc3c(cc2c1)oc1cc2oc4cc5cc(N(c6ccc(-c7ccccc7)cc6)c6c(C)cccc6C)ccc5cc4c2cc13 ; Cc1cccc(C)c1N(c1ccc2cc3c(cc2c1)oc1cc2oc4cc5cc(N(c6ccc(-c7ccccc7)cc6-c6ccccc6)c6c(C)cccc6C)ccc5cc4c2cc13)c1ccc(-c2ccccc2)cc1-c1ccccc1 ; Cc1ccc(-c2ccccc2)cc1N(c1ccc2cc3c(cc2c1)oc1cc2oc4cc5cc(N(c6cc(-c7ccccc7)ccc6C)c6c(C)cccc6C)ccc5cc4c2cc13)c1c(C)cccc1C ; Cc1ccccc1N(c1ccc2cc3c(cc2c1)oc1cc2oc4cc5cc(N(c6ccccc6C)c6ccc(-c7ccccc7)cc6C)ccc5cc4c2cc13)c1ccc(-c2ccccc2)cc1C]

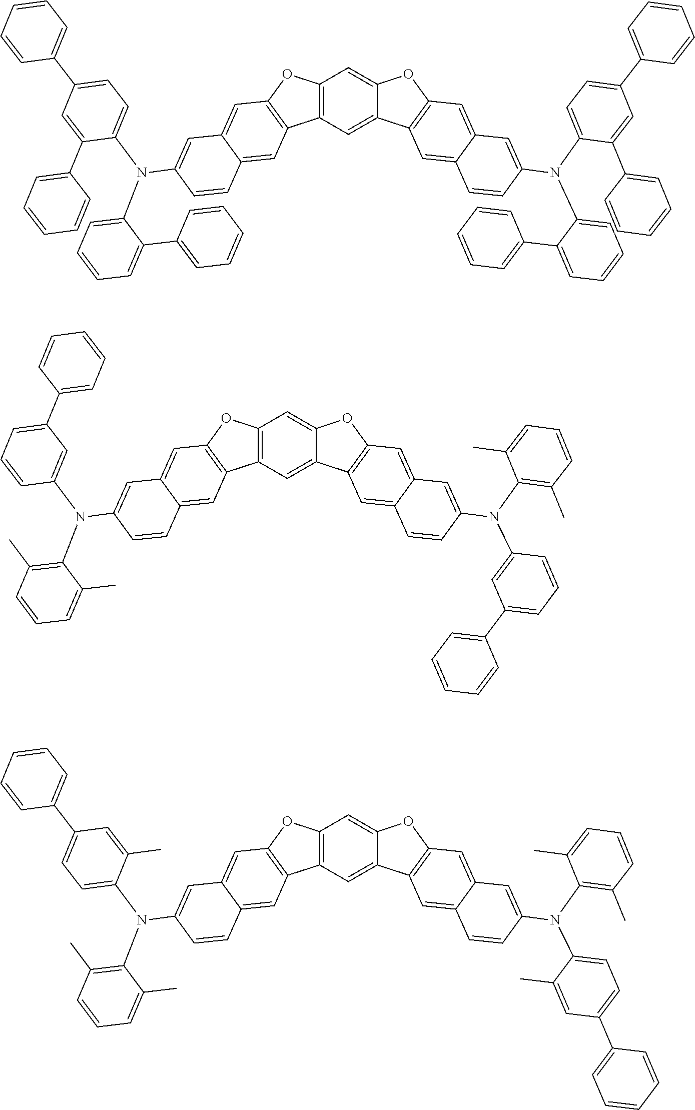 [SMILES: c1ccc(-c2ccc(N(c3ccc4cc5c(cc4c3)oc3cc4oc6cc7cc(N(c8ccccc8-c8ccccc8)c8ccc(-c9ccccc9)cc8-c8ccccc8)ccc7cc6c4cc35)c3ccccc3-c3ccccc3)c(-c3ccccc3)c2)cc1 ; Cc1cccc(C)c1N(c1cccc(-c2ccccc2)c1)c1ccc2cc3c(cc2c1)oc1cc2oc4cc5cc(N(c6cccc(-c7ccccc7)c6)c6c(C)cccc6C)ccc5cc4c2cc13 ; Cc1cc(-c2ccccc2)ccc1N(c1ccc2cc3c(cc2c1)oc1cc2oc4cc5cc(N(c6ccc(-c7ccccc7)cc6C)c6c(C)cccc6C)ccc5cc4c2cc13)c1c(C)cccc1C]

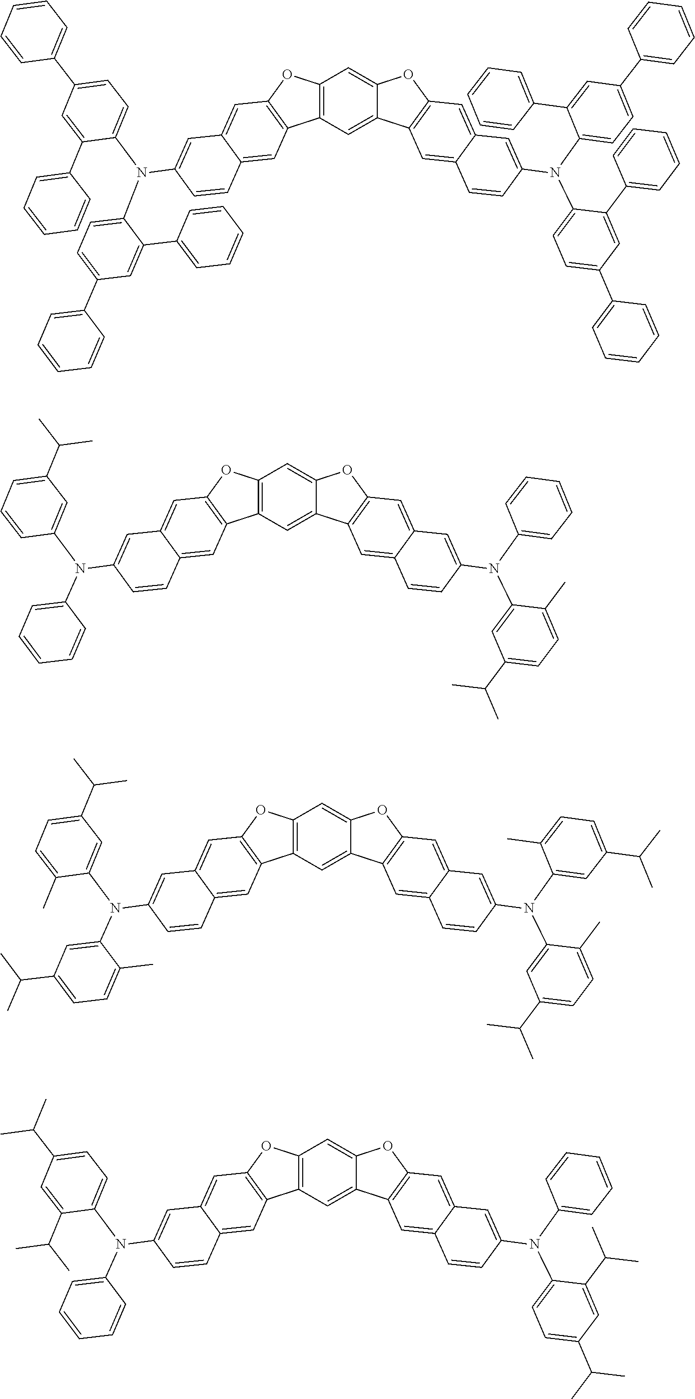

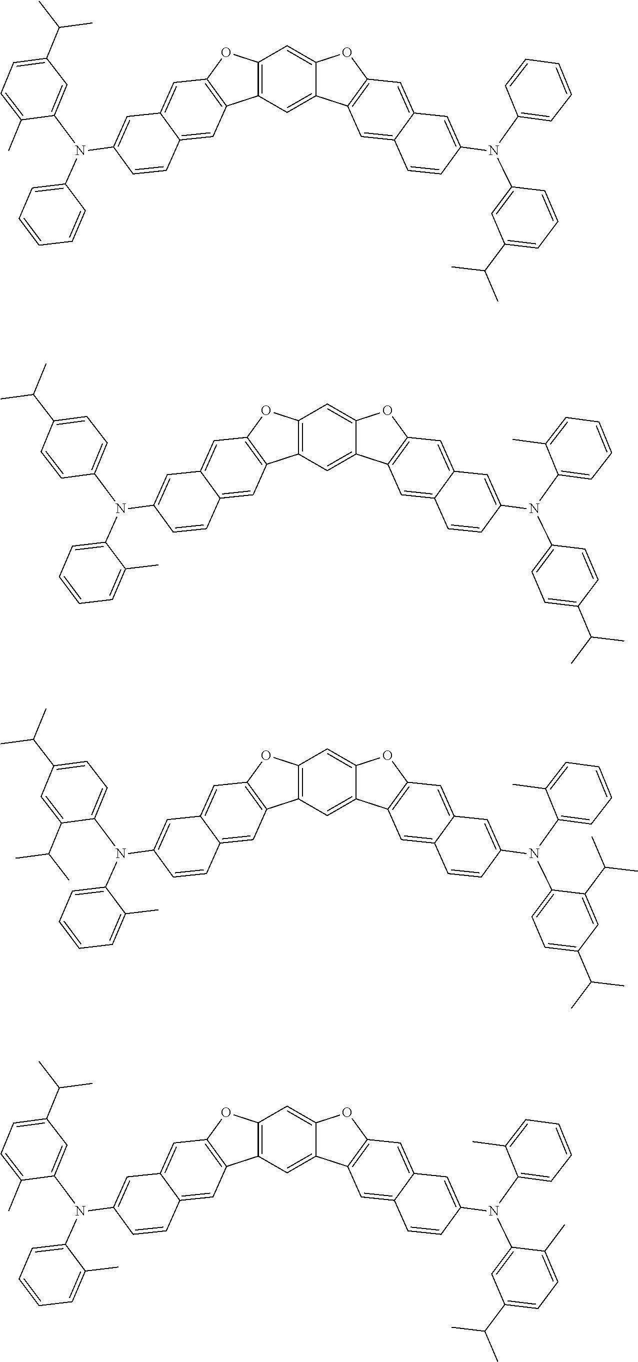

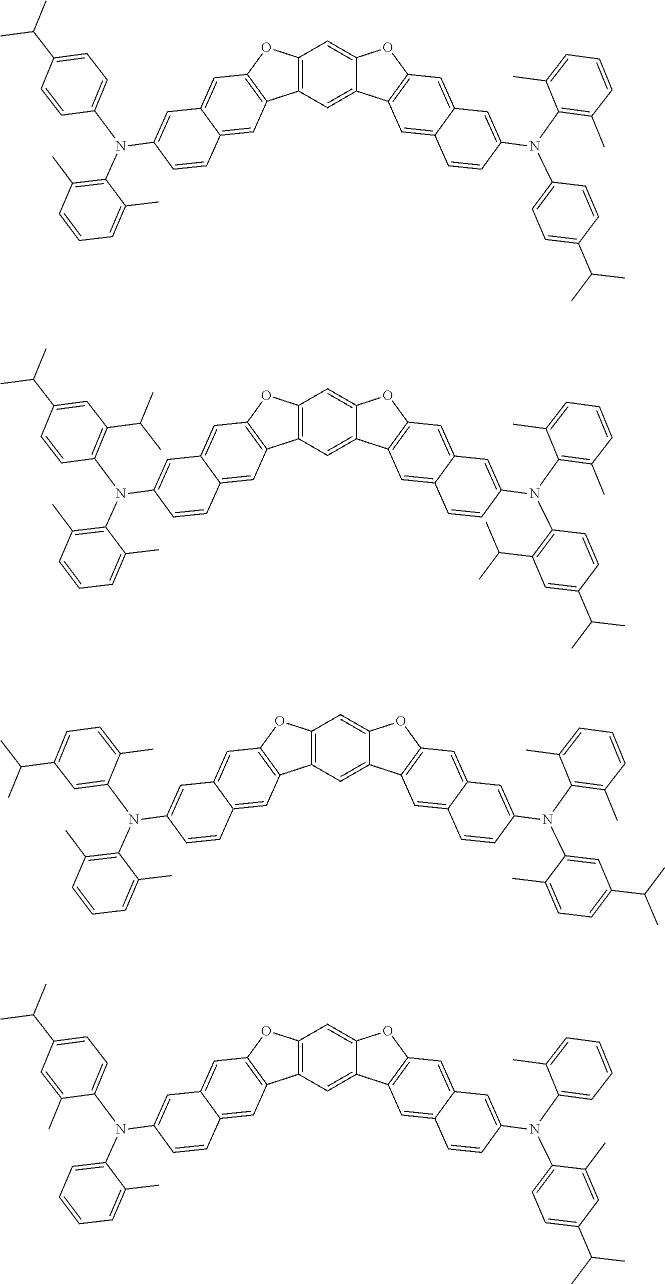

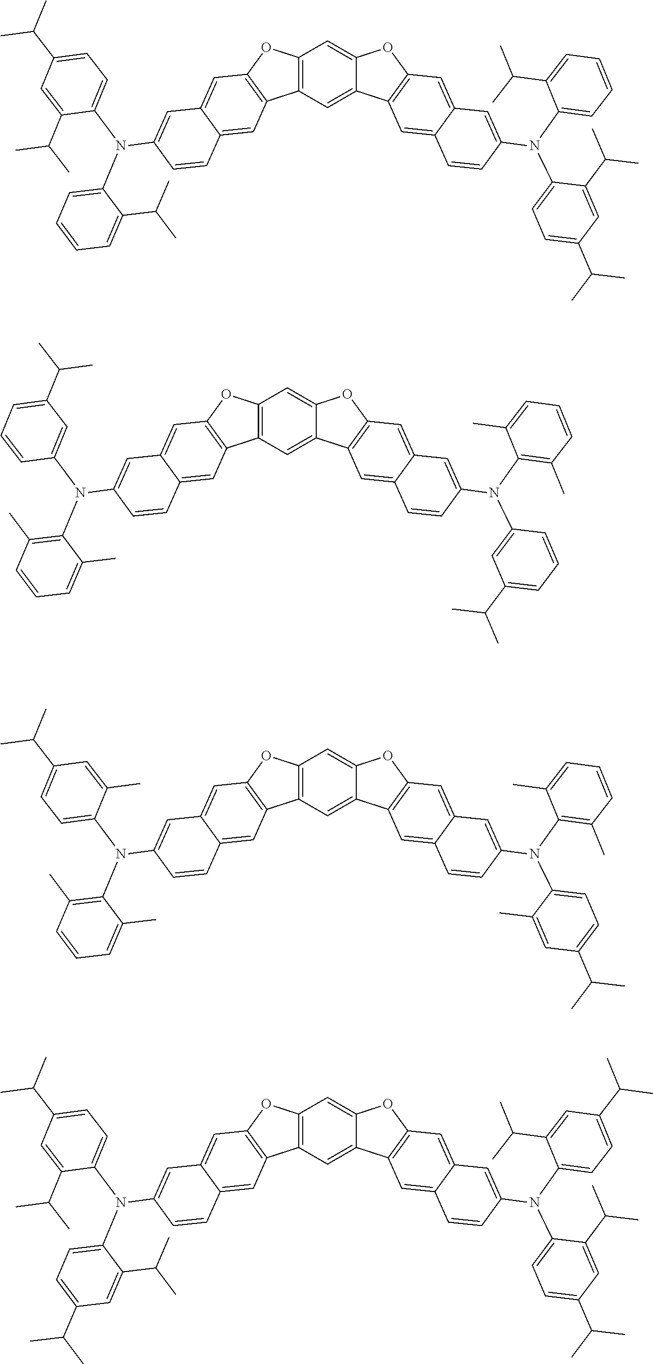

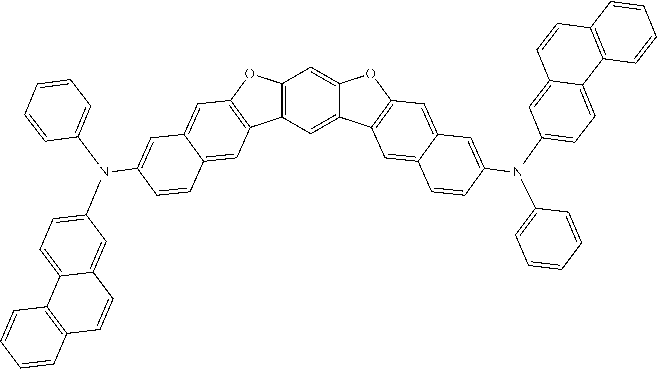 [SMILES: c1ccc(N(c2ccc3cc4c(cc3c2)oc2cc3oc5cc6cc(N(c7ccccc7)c7ccc8c(ccc9ccccc98)c7)ccc6cc5c3cc24)c2ccc3c(ccc4ccccc43)c2)cc1]

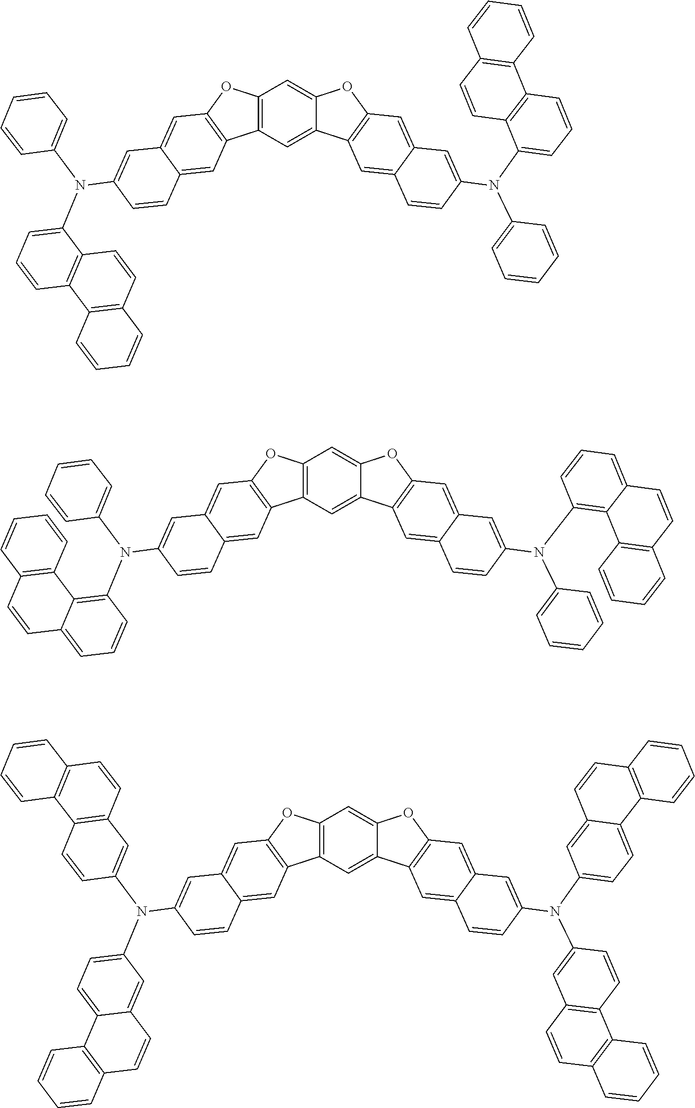 [SMILES: c1ccc(N(c2ccc3cc4c(cc3c2)oc2cc3oc5cc6cc(N(c7ccccc7)c7cccc8c7ccc7ccccc78)ccc6cc5c3cc24)c2cccc3c2ccc2ccccc23)cc1 ; c1ccc(N(c2ccc3cc4c(cc3c2)oc2cc3oc5cc6cc(N(c7ccccc7)c7cccc8ccc9ccccc9c78)ccc6cc5c3cc24)c2cccc3ccc4ccccc4c23)cc1 ; c1ccc2c(c1)ccc1cc(N(c3ccc4cc5c(cc4c3)oc3cc4oc6cc7cc(N(c8ccc9c(ccc%10ccccc%109)c8)c8ccc9c(ccc%10ccccc%109)c8)ccc7cc6c4cc35)c3ccc4c(ccc5ccccc54)c3)ccc12]

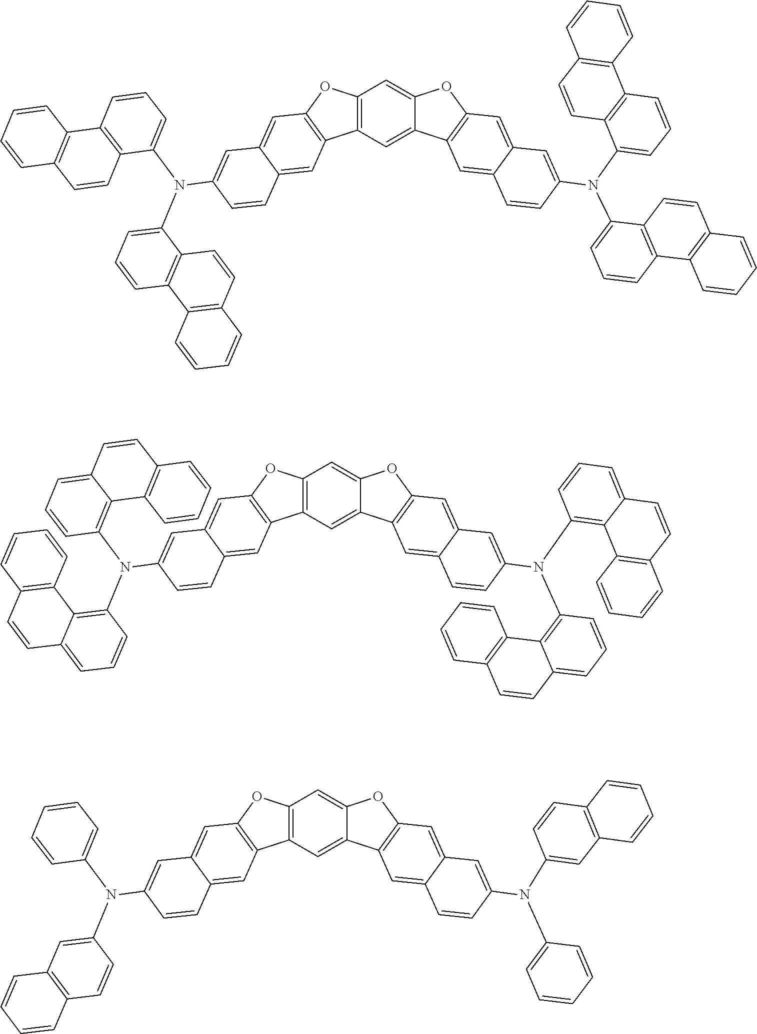 [SMILES: c1ccc2c(c1)ccc1c(N(c3ccc4cc5c(cc4c3)oc3cc4oc6cc7cc(N(c8cccc9c8ccc8ccccc89)c8cccc9c8ccc8ccccc89)ccc7cc6c4cc35)c3cccc4c3ccc3ccccc34)cccc12 ; c1ccc2c(c1)ccc1cccc(N(c3ccc4cc5c(cc4c3)oc3cc4oc6cc7cc(N(c8cccc9ccc%10ccccc%10c89)c8cccc9ccc%10ccccc%10c89)ccc7cc6c4cc35)c3cccc4ccc5ccccc5c34)c12 ; c1ccc(N(c2ccc3ccccc3c2)c2ccc3cc4c(cc3c2)oc2cc3oc5cc6cc(N(c7ccccc7)c7ccc8ccccc8c7)ccc6cc5c3cc24)cc1]

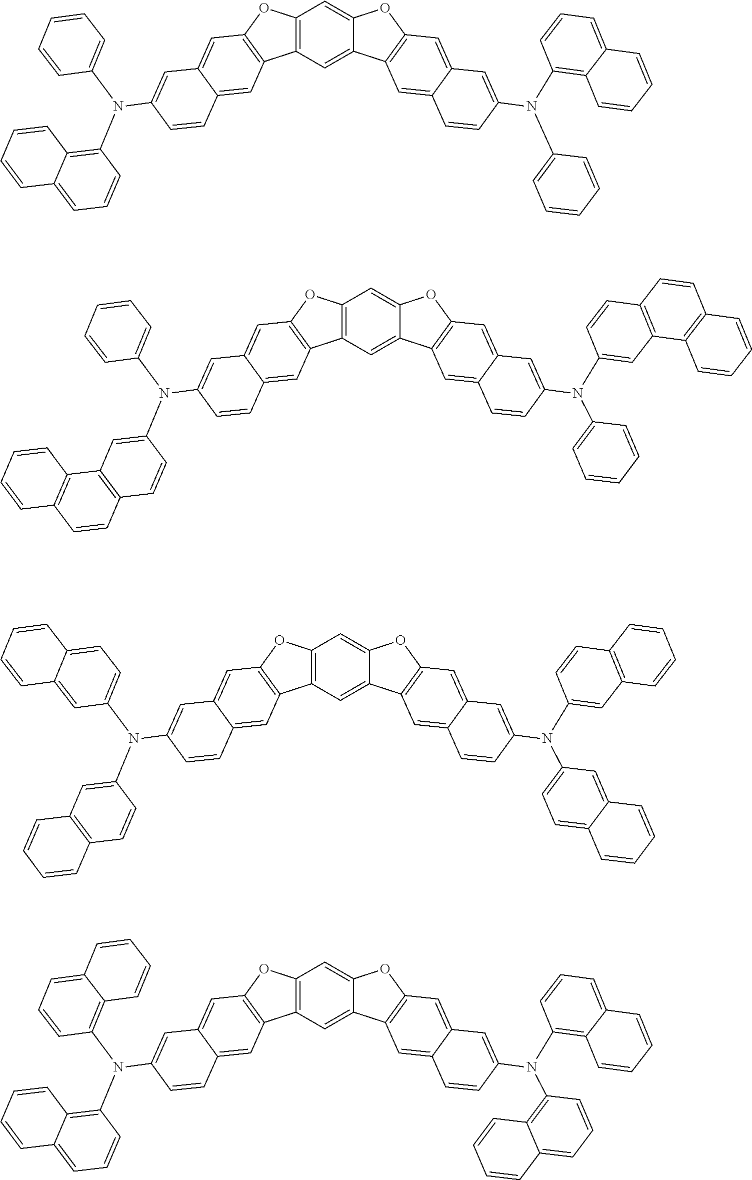 [SMILES: c1ccc(N(c2ccc3cc4c(cc3c2)oc2cc3oc5cc6cc(N(c7ccccc7)c7cccc8ccccc78)ccc6cc5c3cc24)c2cccc3ccccc23)cc1 ; c1ccc(N(c2ccc3cc4c(cc3c2)oc2cc3oc5cc6cc(N(c7ccccc7)c7ccc8ccc9ccccc9c8c7)ccc6cc5c3cc24)c2ccc3ccc4ccccc4c3c2)cc1 ; c1ccc2cc(N(c3ccc4ccccc4c3)c3ccc4cc5c(cc4c3)oc3cc4oc6cc7cc(N(c8ccc9ccccc9c8)c8ccc9ccccc9c8)ccc7cc6c4cc35)ccc2c1 ; c1ccc2c(N(c3ccc4cc5c(cc4c3)oc3cc4oc6cc7cc(N(c8cccc9ccccc89)c8cccc9ccccc89)ccc7cc6c4cc35)c3cccc4ccccc34)cccc2c1]

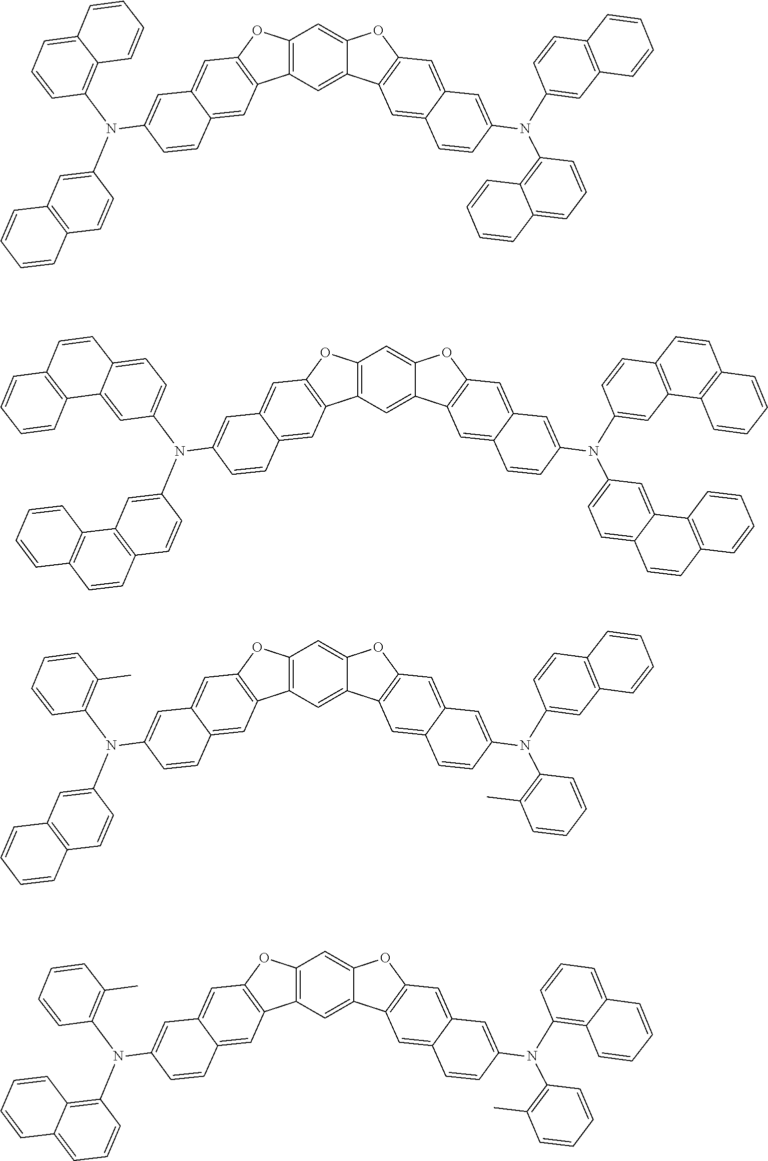 [SMILES: c1ccc2cc(N(c3ccc4cc5c(cc4c3)oc3cc4oc6cc7cc(N(c8ccc9ccccc9c8)c8cccc9ccccc89)ccc7cc6c4cc35)c3cccc4ccccc34)ccc2c1 ; c1ccc2c(c1)ccc1ccc(N(c3ccc4cc5c(cc4c3)oc3cc4oc6cc7cc(N(c8ccc9ccc%10ccccc%10c9c8)c8ccc9ccc%10ccccc%10c9c8)ccc7cc6c4cc35)c3ccc4ccc5ccccc5c4c3)cc12 ; Cc1ccccc1N(c1ccc2ccccc2c1)c1ccc2cc3c(cc2c1)oc1cc2oc4cc5cc(N(c6ccc7ccccc7c6)c6ccccc6C)ccc5cc4c2cc13 ; Cc1ccccc1N(c1ccc2cc3c(cc2c1)oc1cc2oc4cc5cc(N(c6ccccc6C)c6cccc7ccccc67)ccc5cc4c2cc13)c1cccc2ccccc12]

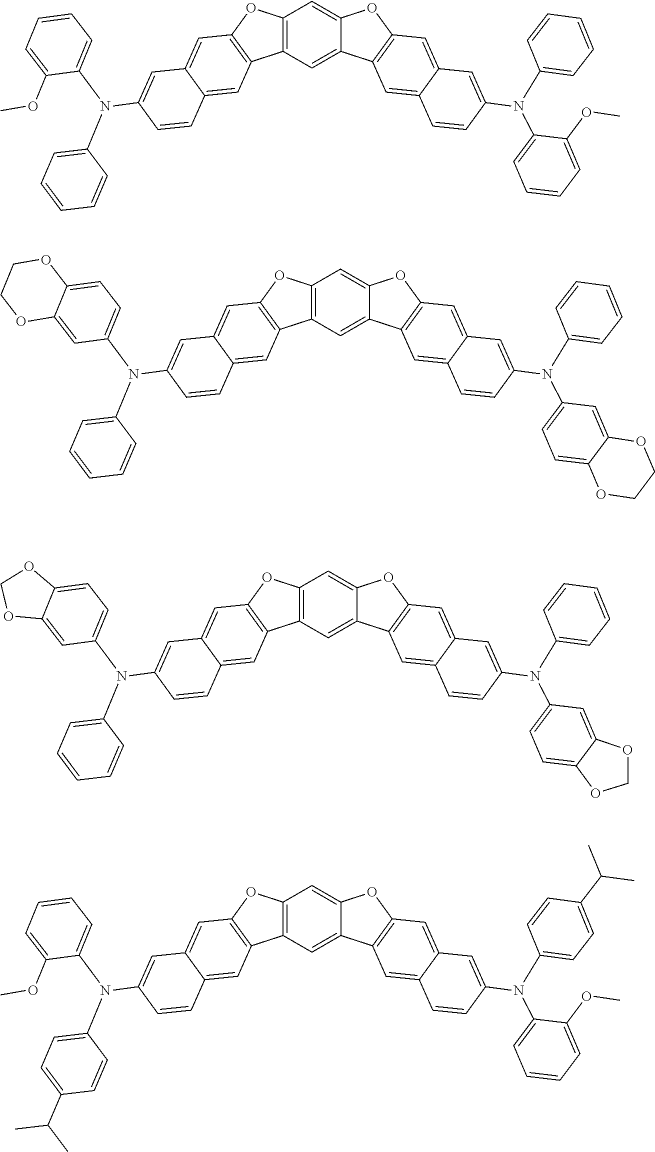

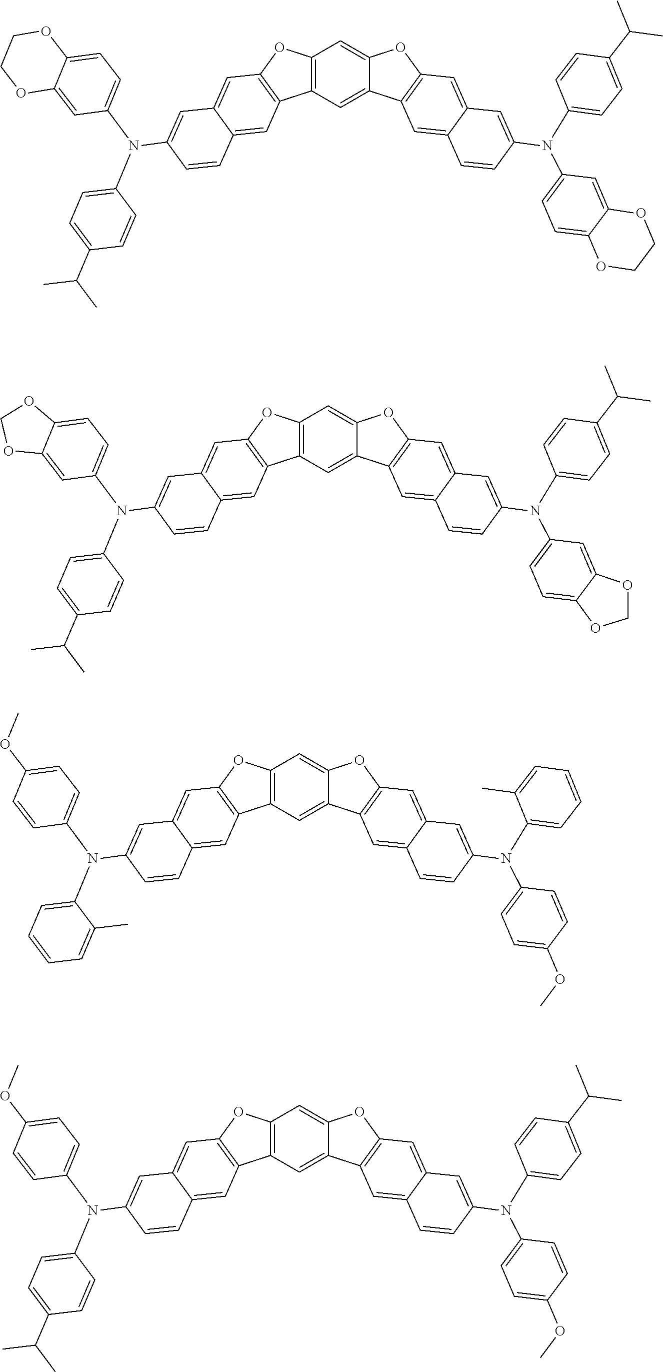

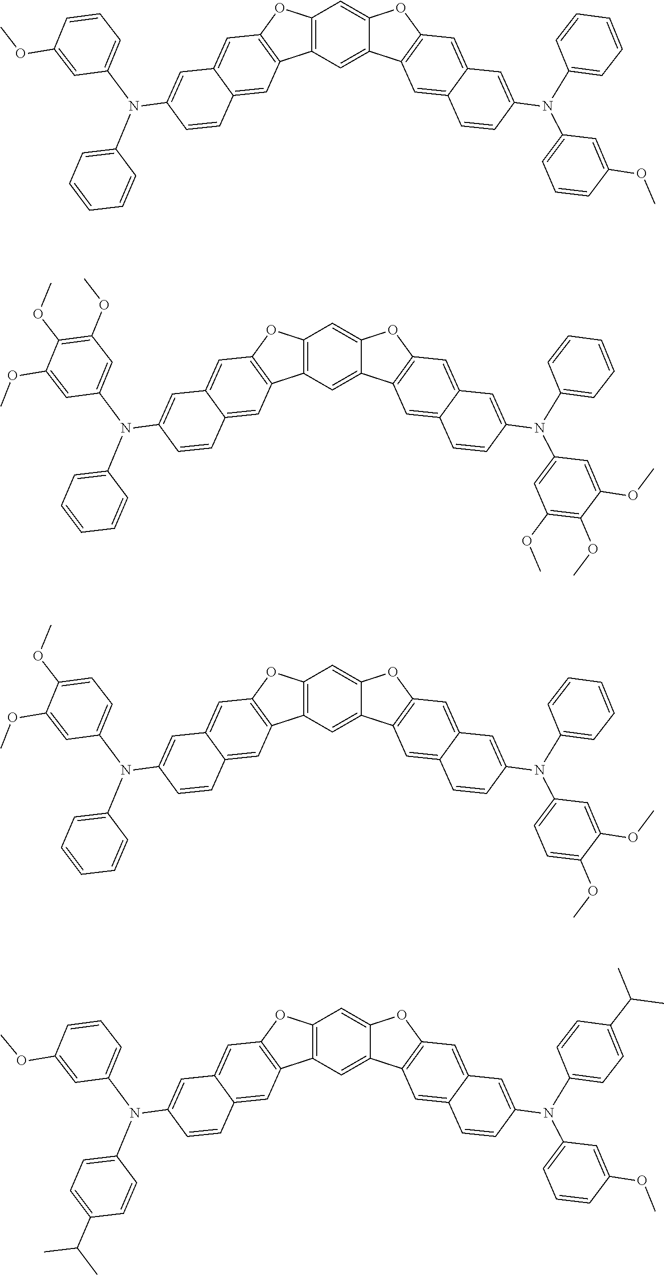

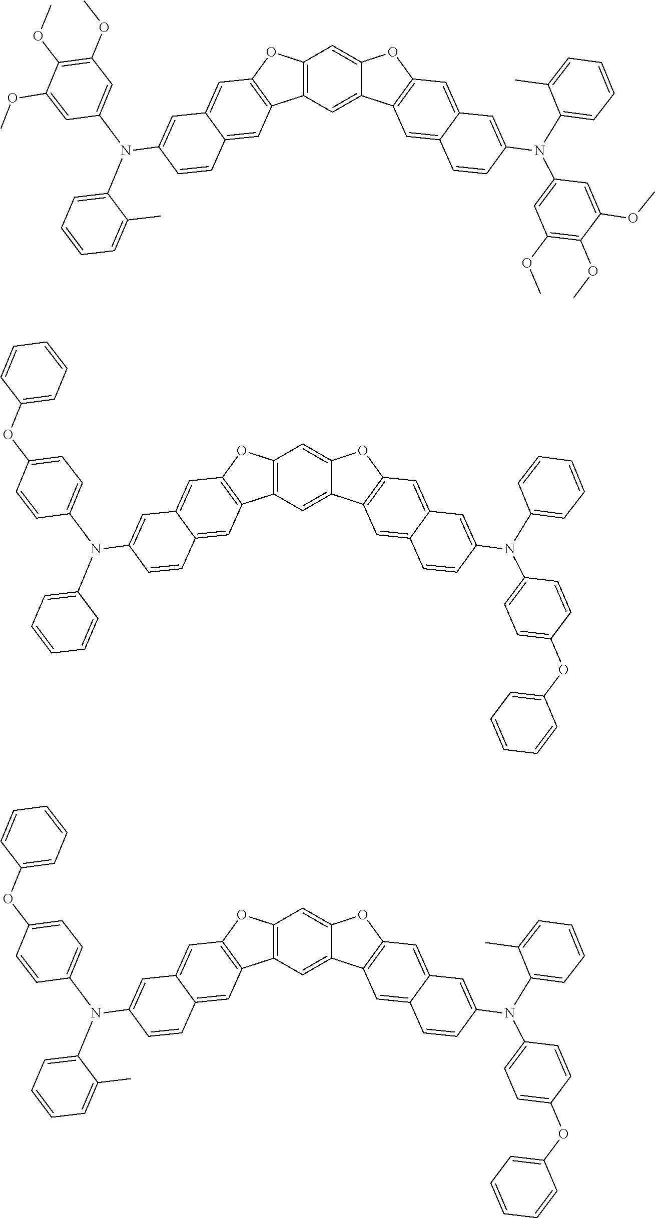

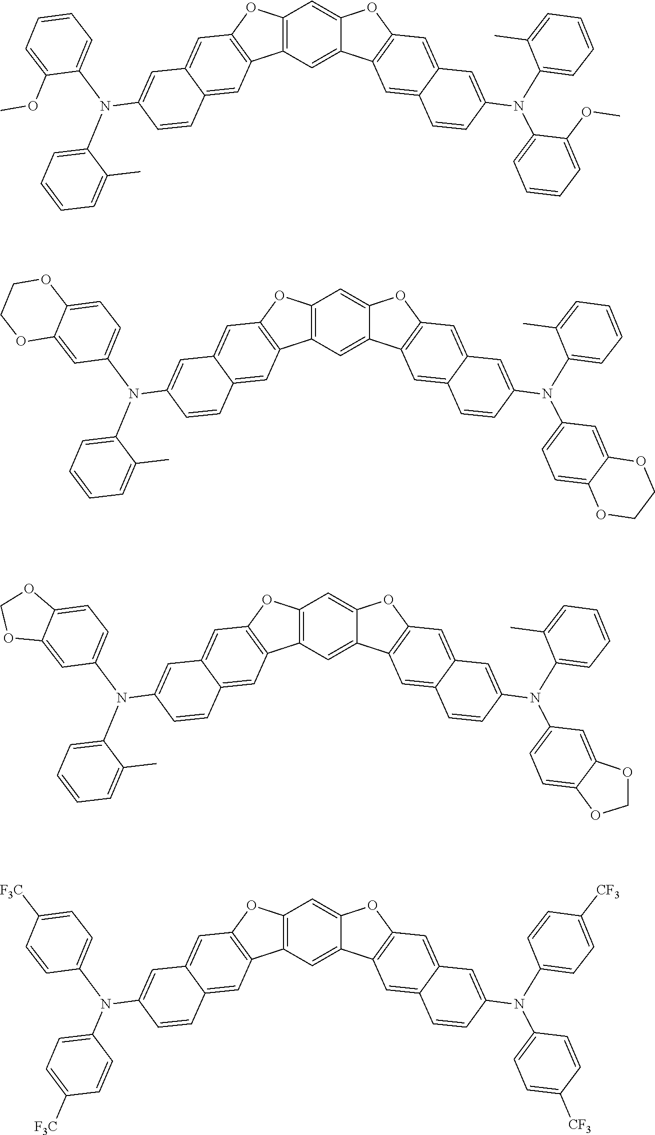

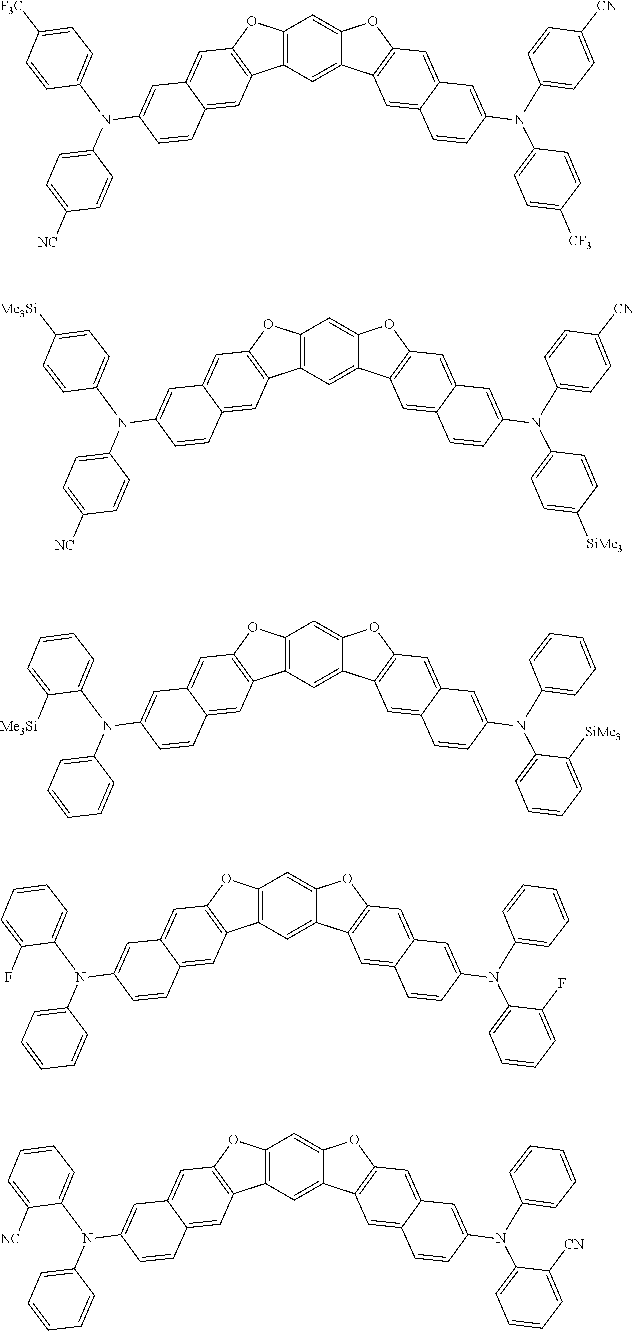

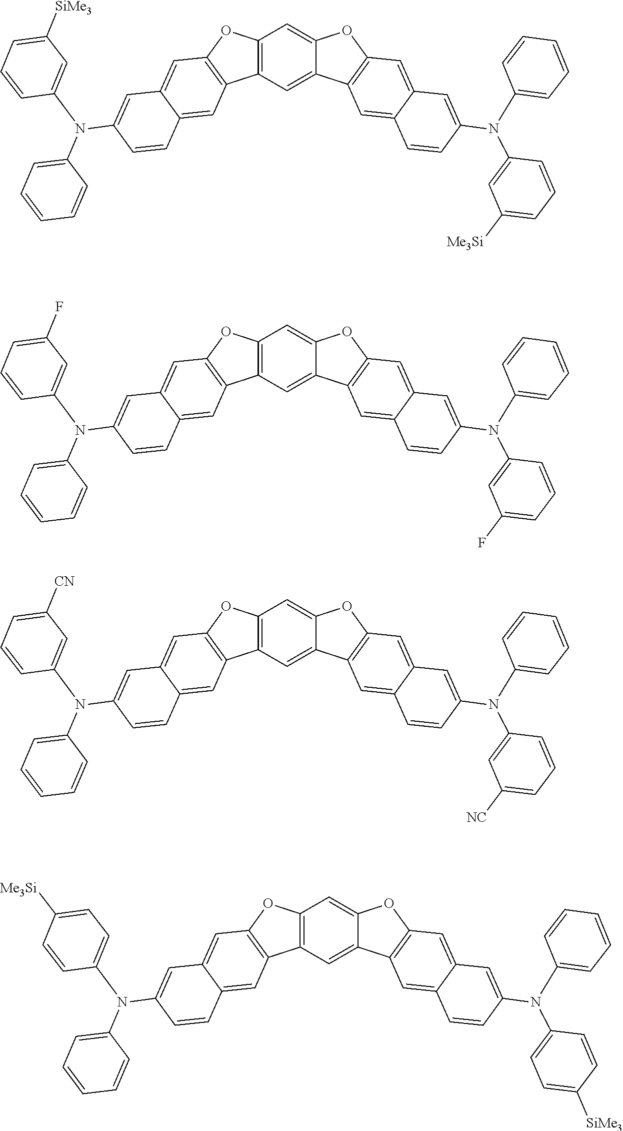 [SMILES: C[Si](C)(C)c1cccc(N(c2ccccc2)c2ccc3cc4c(cc3c2)oc2cc3oc5cc6cc(N(c7ccccc7)c7cccc([Si](C)(C)C)c7)ccc6cc5c3cc24)c1 ; Fc1cccc(N(c2ccccc2)c2ccc3cc4c(cc3c2)oc2cc3oc5cc6cc(N(c7ccccc7)c7cccc(F)c7)ccc6cc5c3cc24)c1 ; N#Cc1cccc(N(c2ccccc2)c2ccc3cc4c(cc3c2)oc2cc3oc5cc6cc(N(c7ccccc7)c7cccc(C#N)c7)ccc6cc5c3cc24)c1 ; C[Si](C)(C)c1ccc(N(c2ccccc2)c2ccc3cc4c(cc3c2)oc2cc3oc5cc6cc(N(c7ccccc7)c7ccc([Si](C)(C)C)cc7)ccc6cc5c3cc24)cc1]

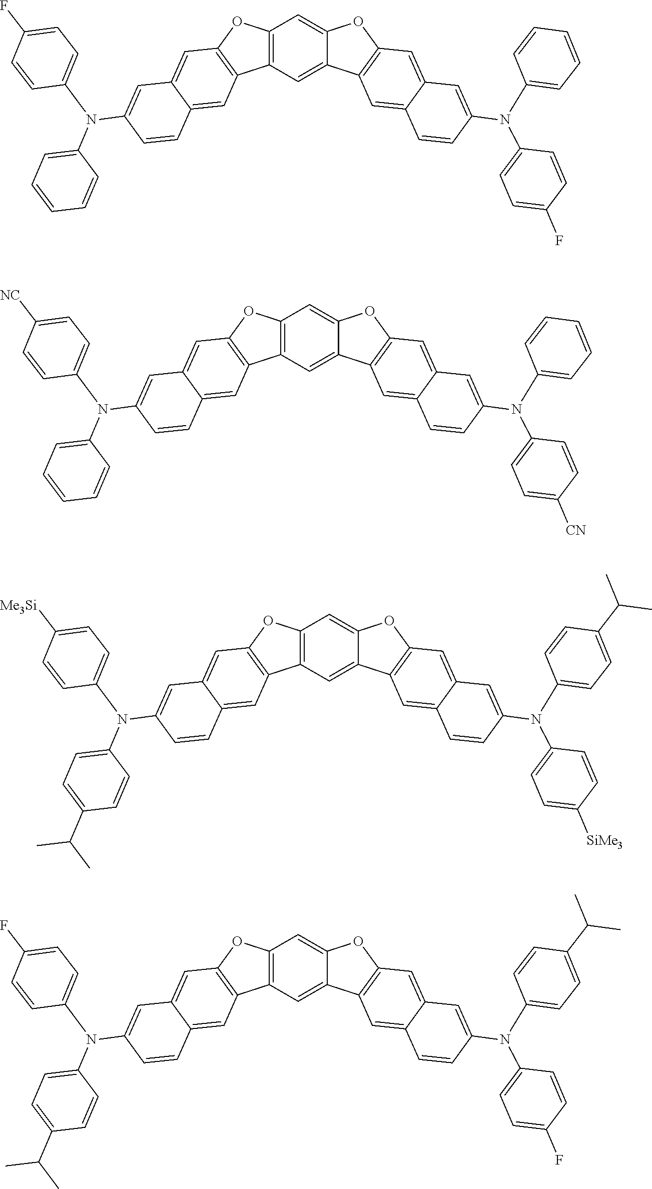 [SMILES: Fc1ccc(N(c2ccccc2)c2ccc3cc4c(cc3c2)oc2cc3oc5cc6cc(N(c7ccccc7)c7ccc(F)cc7)ccc6cc5c3cc24)cc1 ; N#Cc1ccc(N(c2ccccc2)c2ccc3cc4c(cc3c2)oc2cc3oc5cc6cc(N(c7ccccc7)c7ccc(C#N)cc7)ccc6cc5c3cc24)cc1 ; CC(C)c1ccc(N(c2ccc([Si](C)(C)C)cc2)c2ccc3cc4c(cc3c2)oc2cc3oc5cc6cc(N(c7ccc(C(C)C)cc7)c7ccc([Si](C)(C)C)cc7)ccc6cc5c3cc24)cc1 ; CC(C)c1ccc(N(c2ccc(F)cc2)c2ccc3cc4c(cc3c2)oc2cc3oc5cc6cc(N(c7ccc(F)cc7)c7ccc(C(C)C)cc7)ccc6cc5c3cc24)cc1]

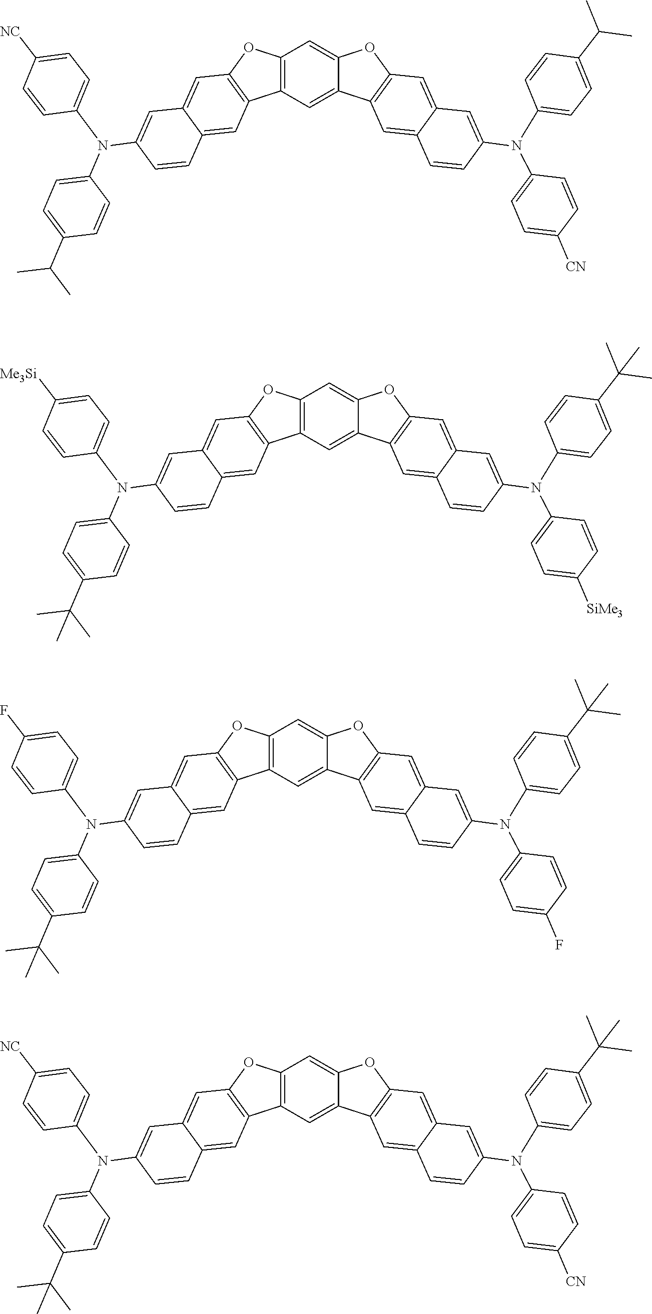 [SMILES: CC(C)c1ccc(N(c2ccc(C#N)cc2)c2ccc3cc4c(cc3c2)oc2cc3oc5cc6cc(N(c7ccc(C#N)cc7)c7ccc(C(C)C)cc7)ccc6cc5c3cc24)cc1 ; CC(C)(C)c1ccc(N(c2ccc([Si](C)(C)C)cc2)c2ccc3cc4c(cc3c2)oc2cc3oc5cc6cc(N(c7ccc(C(C)(C)C)cc7)c7ccc([Si](C)(C)C)cc7)ccc6cc5c3cc24)cc1 ; CC(C)(C)c1ccc(N(c2ccc(F)cc2)c2ccc3cc4c(cc3c2)oc2cc3oc5cc6cc(N(c7ccc(F)cc7)c7ccc(C(C)(C)C)cc7)ccc6cc5c3cc24)cc1 ; CC(C)(C)c1ccc(N(c2ccc(C#N)cc2)c2ccc3cc4c(cc3c2)oc2cc3oc5cc6cc(N(c7ccc(C#N)cc7)c7ccc(C(C)(C)C)cc7)ccc6cc5c3cc24)cc1]

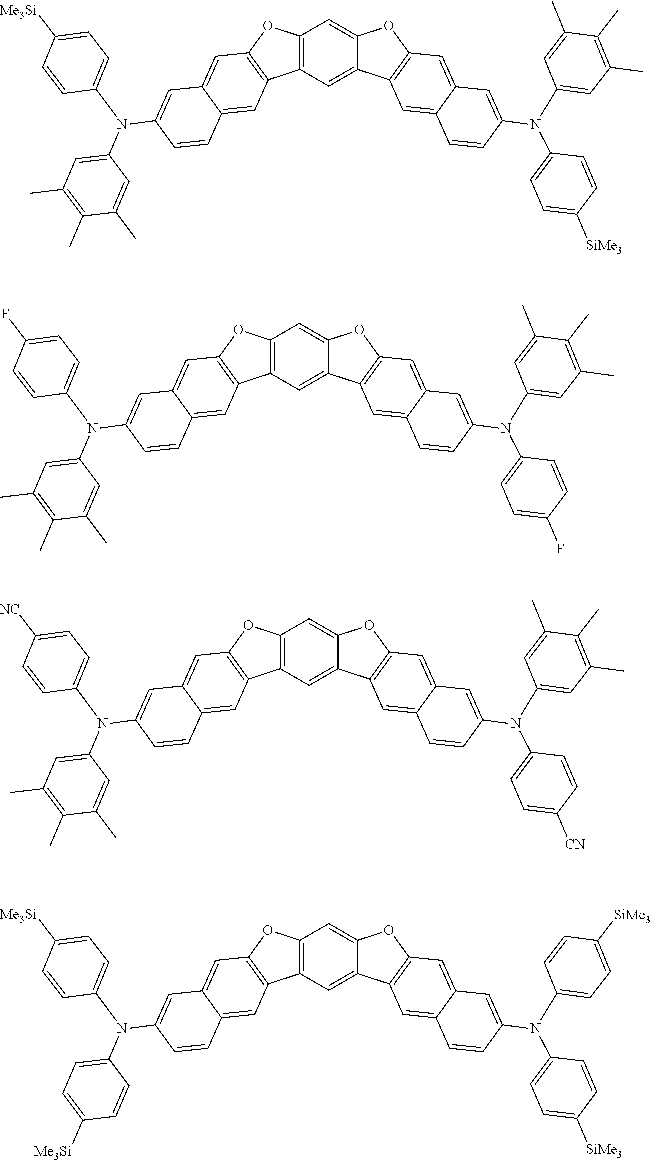

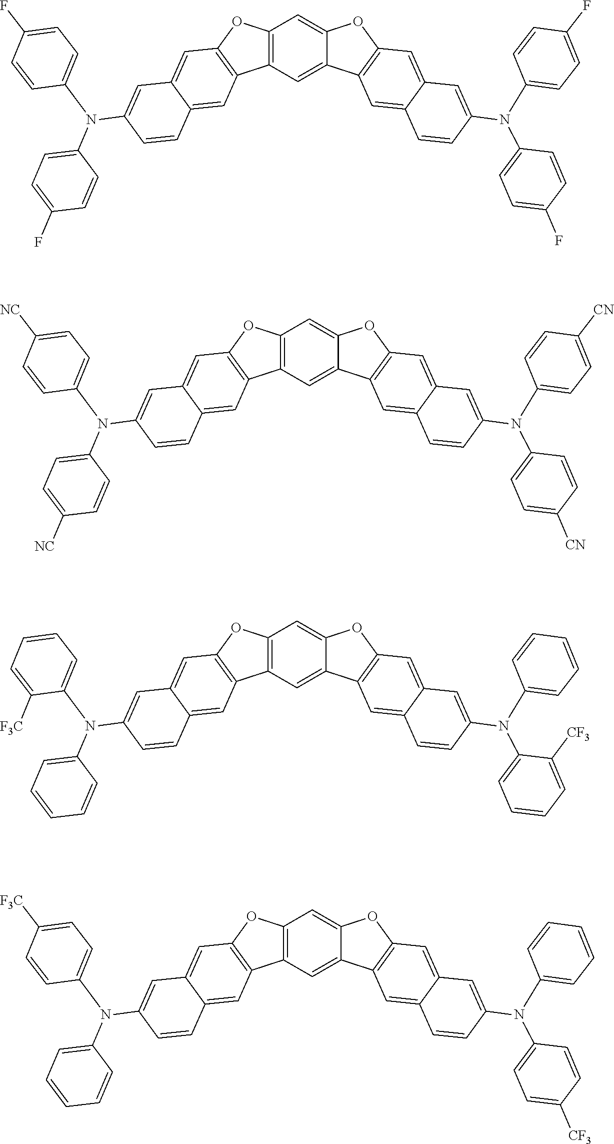 [SMILES: Fc1ccc(N(c2ccc(F)cc2)c2ccc3cc4c(cc3c2)oc2cc3oc5cc6cc(N(c7ccc(F)cc7)c7ccc(F)cc7)ccc6cc5c3cc24)cc1 ; N#Cc1ccc(N(c2ccc(C#N)cc2)c2ccc3cc4c(cc3c2)oc2cc3oc5cc6cc(N(c7ccc(C#N)cc7)c7ccc(C#N)cc7)ccc6cc5c3cc24)cc1 ; FC(F)(F)c1ccccc1N(c1ccccc1)c1ccc2cc3c(cc2c1)oc1cc2oc4cc5cc(N(c6ccccc6)c6ccccc6C(F)(F)F)ccc5cc4c2cc13 ; FC(F)(F)c1ccc(N(c2ccccc2)c2ccc3cc4c(cc3c2)oc2cc3oc5cc6cc(N(c7ccccc7)c7ccc(C(F)(F)F)cc7)ccc6cc5c3cc24)cc1]

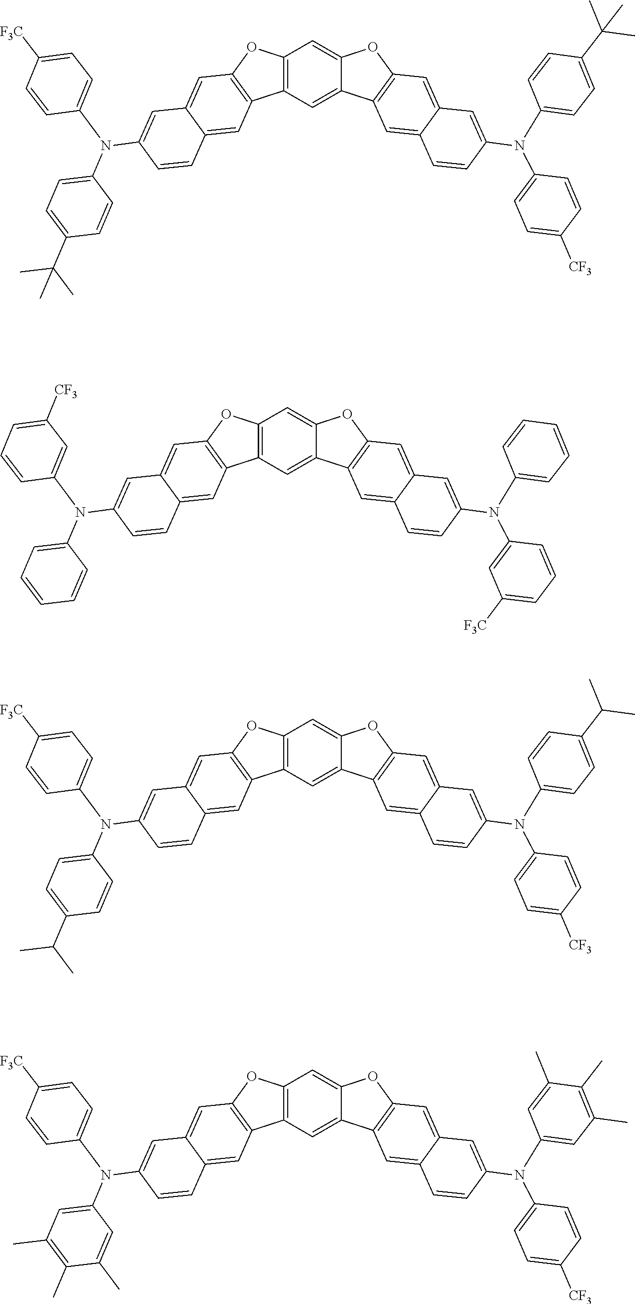

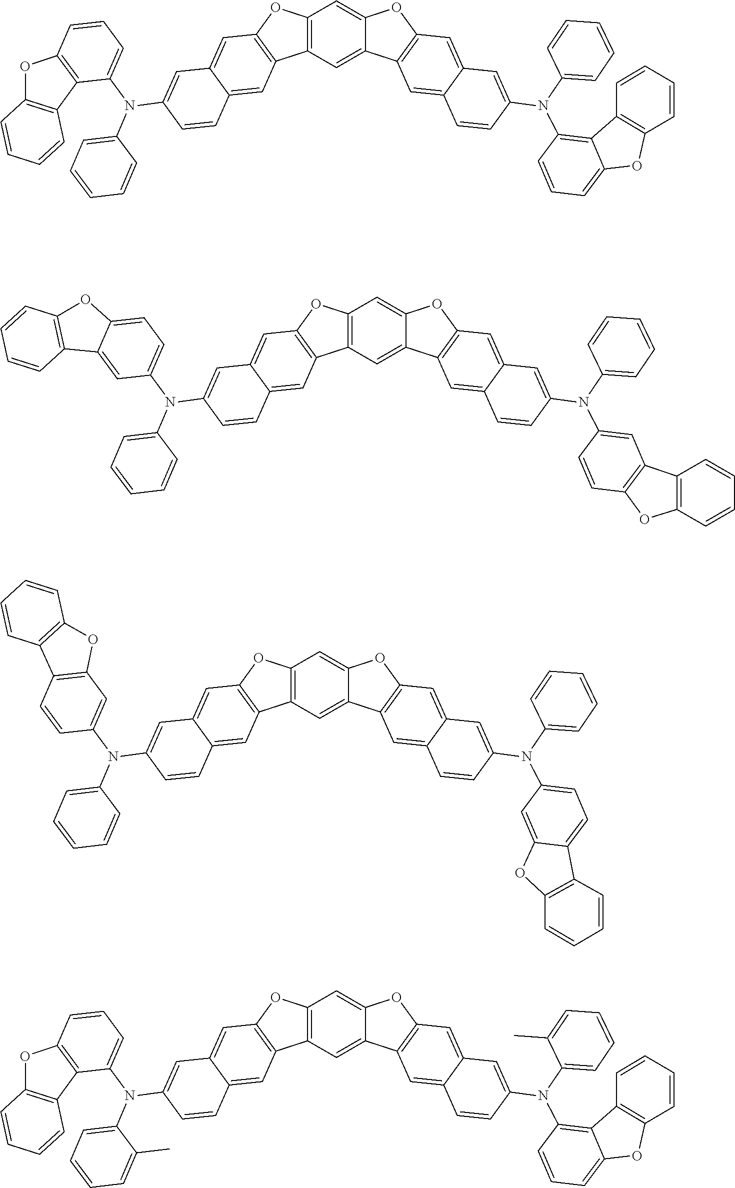 [SMILES: c1ccc(N(c2ccc3cc4c(cc3c2)oc2cc3oc5cc6cc(N(c7ccccc7)c7cccc8oc9ccccc9c78)ccc6cc5c3cc24)c2cccc3oc4ccccc4c23)cc1 ; c1ccc(N(c2ccc3cc4c(cc3c2)oc2cc3oc5cc6cc(N(c7ccccc7)c7ccc8oc9ccccc9c8c7)ccc6cc5c3cc24)c2ccc3oc4ccccc4c3c2)cc1 ; c1ccc(N(c2ccc3cc4c(cc3c2)oc2cc3oc5cc6cc(N(c7ccccc7)c7ccc8c(c7)oc7ccccc78)ccc6cc5c3cc24)c2ccc3c(c2)oc2ccccc23)cc1 ; Cc1ccccc1N(c1ccc2cc3c(cc2c1)oc1cc2oc4cc5cc(N(c6ccccc6C)c6cccc7oc8ccccc8c67)ccc5cc4c2cc13)c1cccc2oc3ccccc3c12]

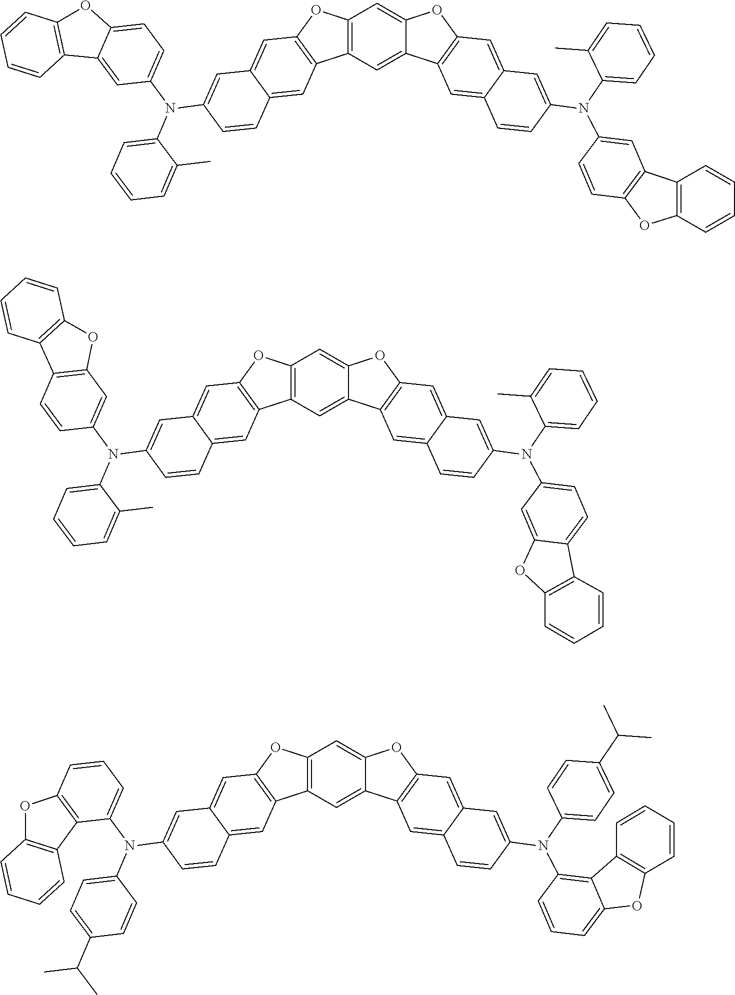 [SMILES: Cc1ccccc1N(c1ccc2cc3c(cc2c1)oc1cc2oc4cc5cc(N(c6ccc7oc8ccccc8c7c6)c6ccccc6C)ccc5cc4c2cc13)c1ccc2oc3ccccc3c2c1 ; Cc1ccccc1N(c1ccc2cc3c(cc2c1)oc1cc2oc4cc5cc(N(c6ccc7c(c6)oc6ccccc67)c6ccccc6C)ccc5cc4c2cc13)c1ccc2c(c1)oc1ccccc12 ; CC(C)c1ccc(N(c2ccc3cc4c(cc3c2)oc2cc3oc5cc6cc(N(c7ccc(C(C)C)cc7)c7cccc8oc9ccccc9c78)ccc6cc5c3cc24)c2cccc3oc4ccccc4c23)cc1]

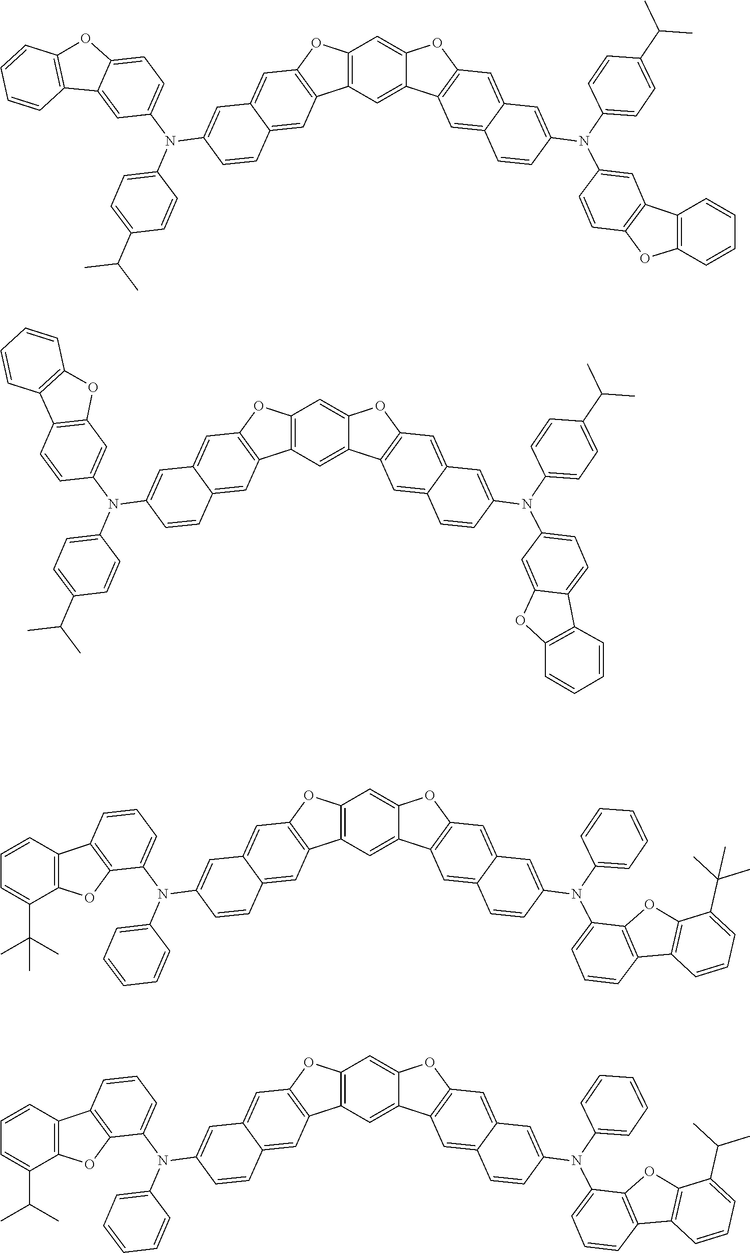 [SMILES: CC(C)c1ccc(N(c2ccc3cc4c(cc3c2)oc2cc3oc5cc6cc(N(c7ccc(C(C)C)cc7)c7ccc8oc9ccccc9c8c7)ccc6cc5c3cc24)c2ccc3oc4ccccc4c3c2)cc1 ; CC(C)c1ccc(N(c2ccc3cc4c(cc3c2)oc2cc3oc5cc6cc(N(c7ccc(C(C)C)cc7)c7ccc8c(c7)oc7ccccc78)ccc6cc5c3cc24)c2ccc3c(c2)oc2ccccc23)cc1 ; CC(C)(C)c1cccc2c1oc1c(N(c3ccccc3)c3ccc4cc5c(cc4c3)oc3cc4oc6cc7cc(N(c8ccccc8)c8cccc9c8oc8c(C(C)(C)C)cccc89)ccc7cc6c4cc35)cccc12 ; CC(C)c1cccc2c1oc1c(N(c3ccccc3)c3ccc4cc5c(cc4c3)oc3cc4oc6cc7cc(N(c8ccccc8)c8cccc9c8oc8c(C(C)C)cccc89)ccc7cc6c4cc35)cccc12]

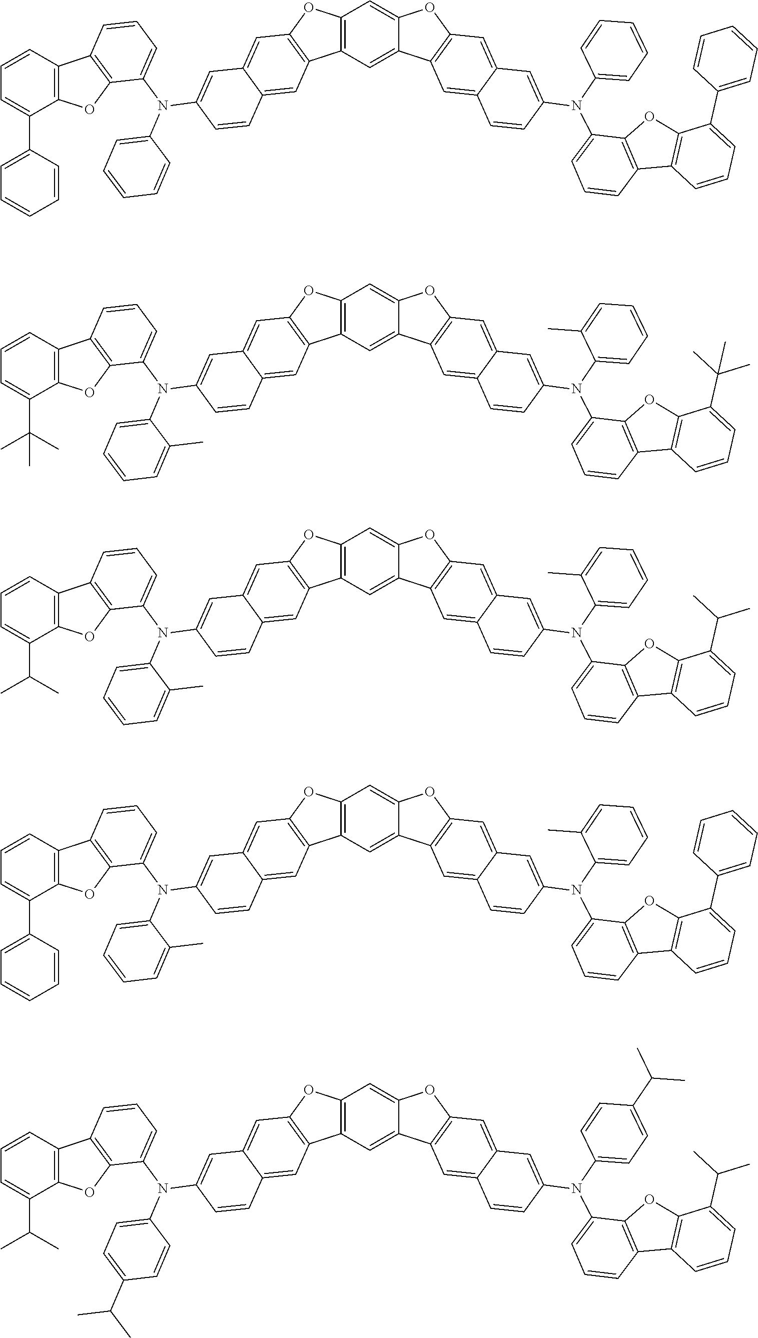

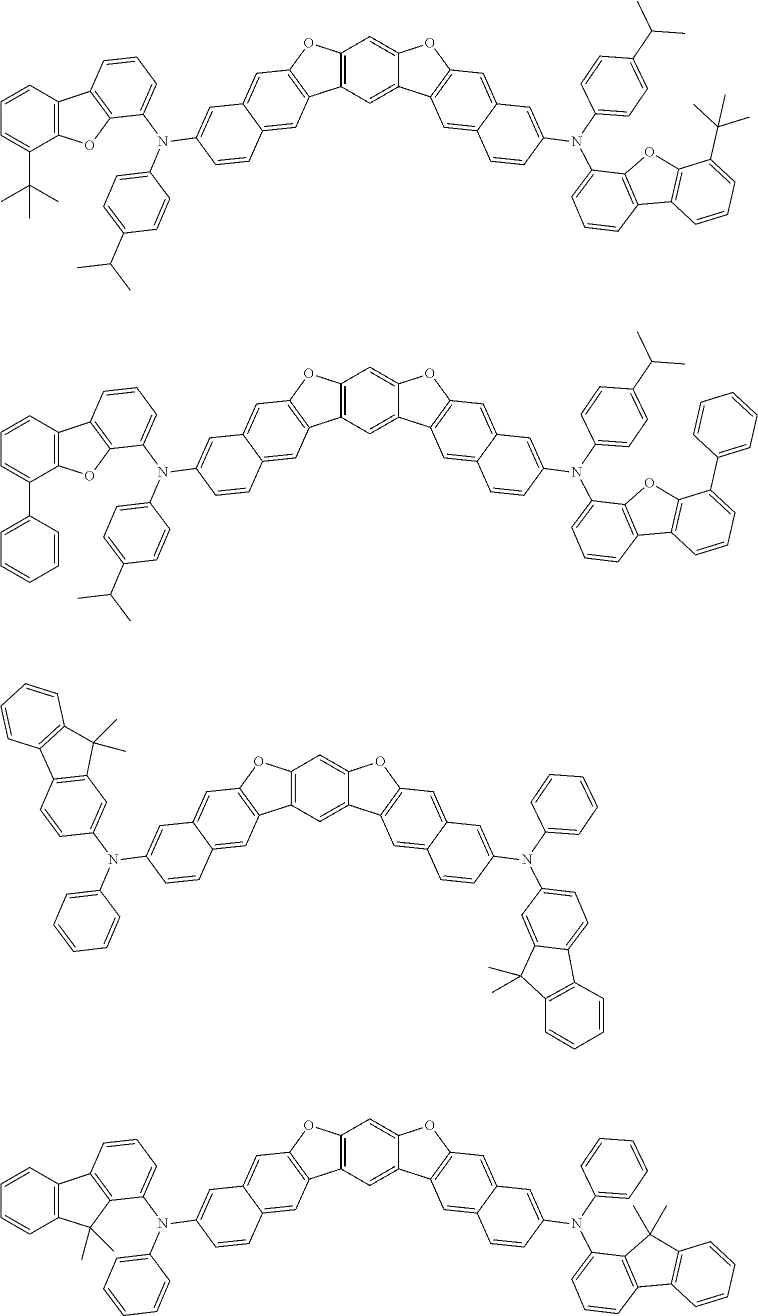

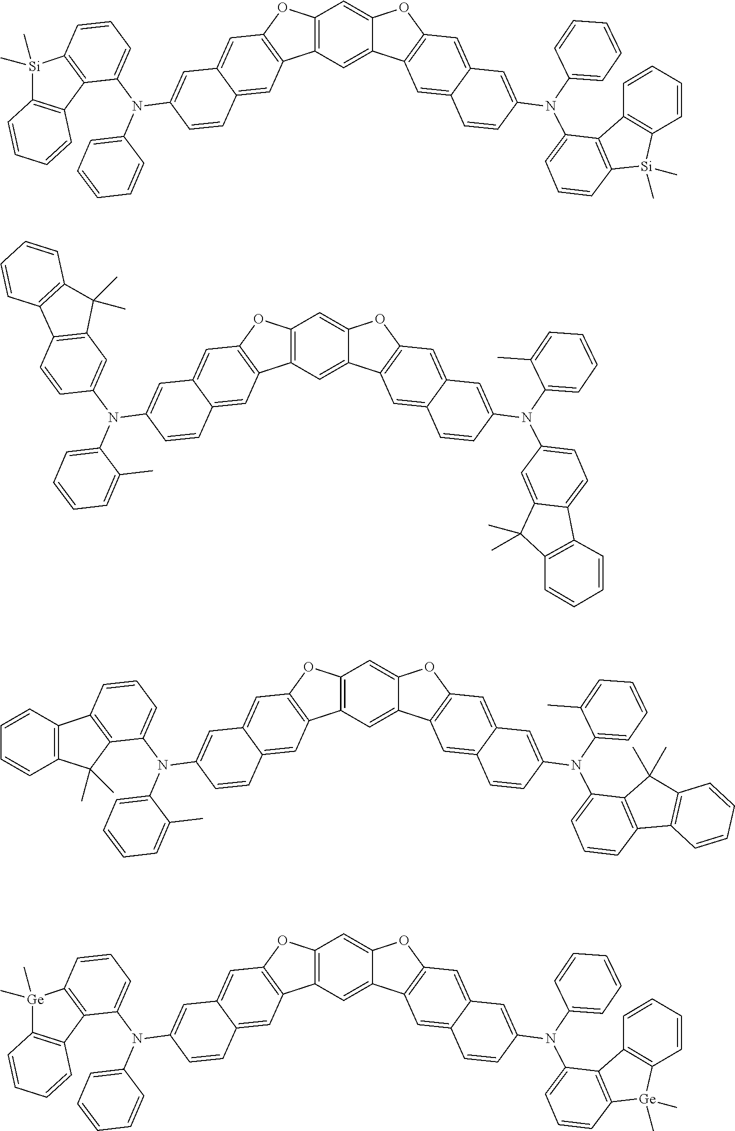

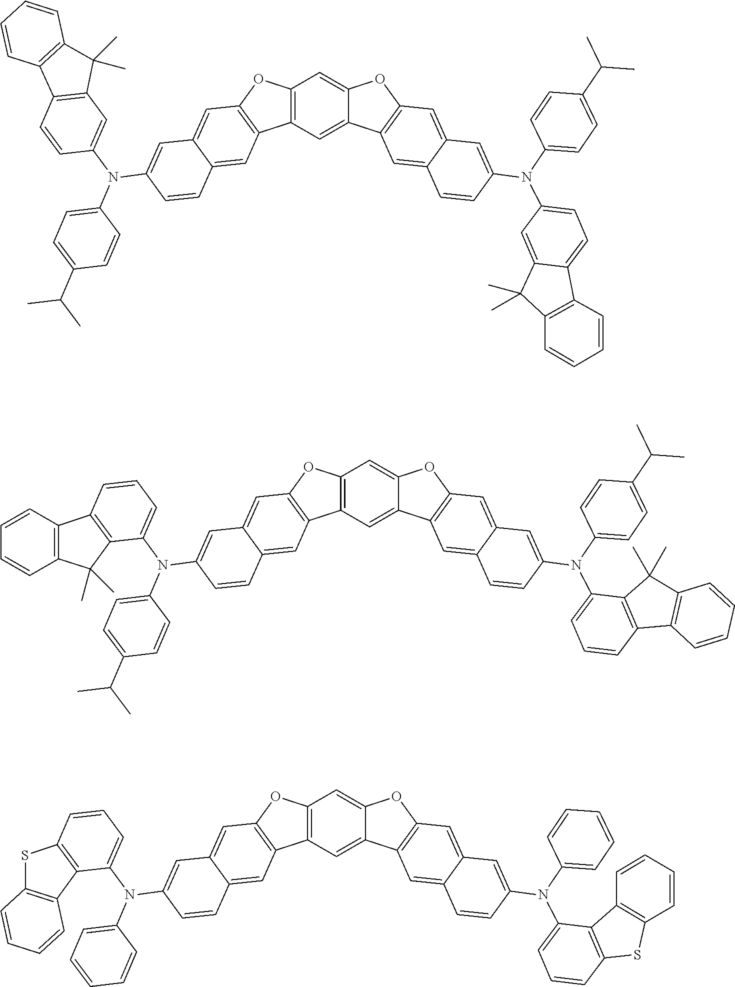 [SMILES: CC(C)c1ccc(N(c2ccc3c(c2)C(C)(C)c2ccccc2-3)c2ccc3cc4c(cc3c2)oc2cc3oc5cc6cc(N(c7ccc(C(C)C)cc7)c7ccc8c(c7)C(C)(C)c7ccccc7-8)ccc6cc5c3cc24)cc1 ; CC(C)c1ccc(N(c2ccc3cc4c(cc3c2)oc2cc3oc5cc6cc(N(c7ccc(C(C)C)cc7)c7cccc8c7C(C)(C)c7ccccc7-8)ccc6cc5c3cc24)c2cccc3c2C(C)(C)c2ccccc2-3)cc1 ; c1ccc(N(c2ccc3cc4c(cc3c2)oc2cc3oc5cc6cc(N(c7ccccc7)c7cccc8sc9ccccc9c78)ccc6cc5c3cc24)c2cccc3sc4ccccc4c23)cc1]

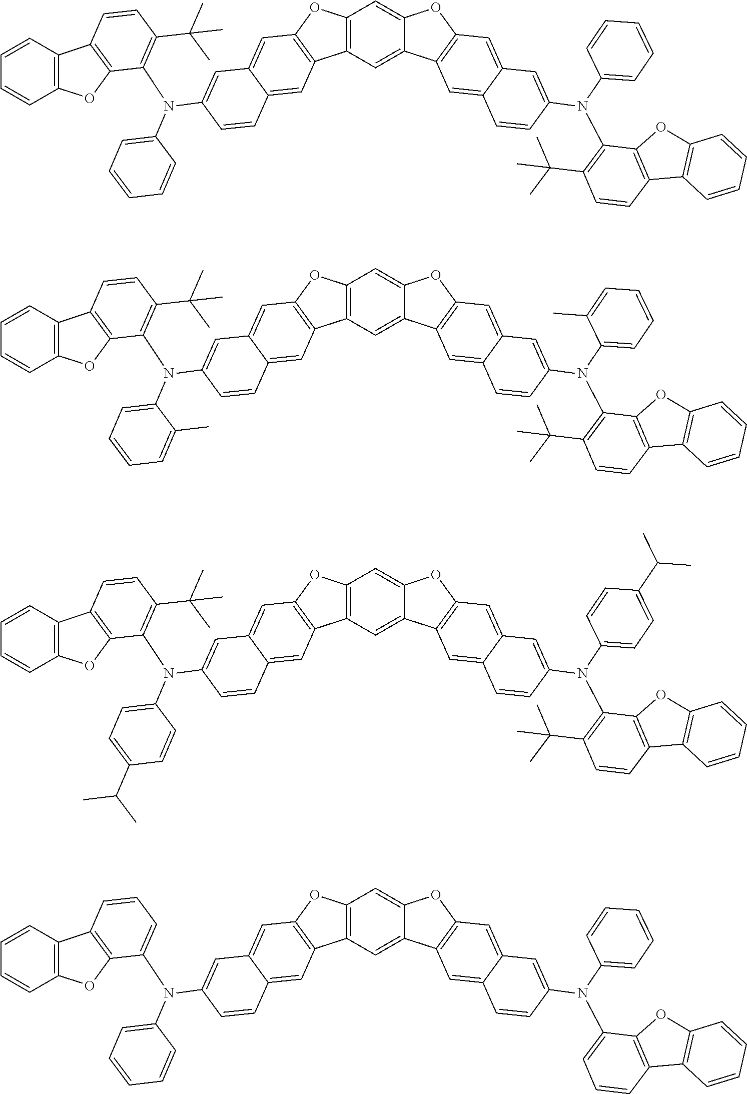

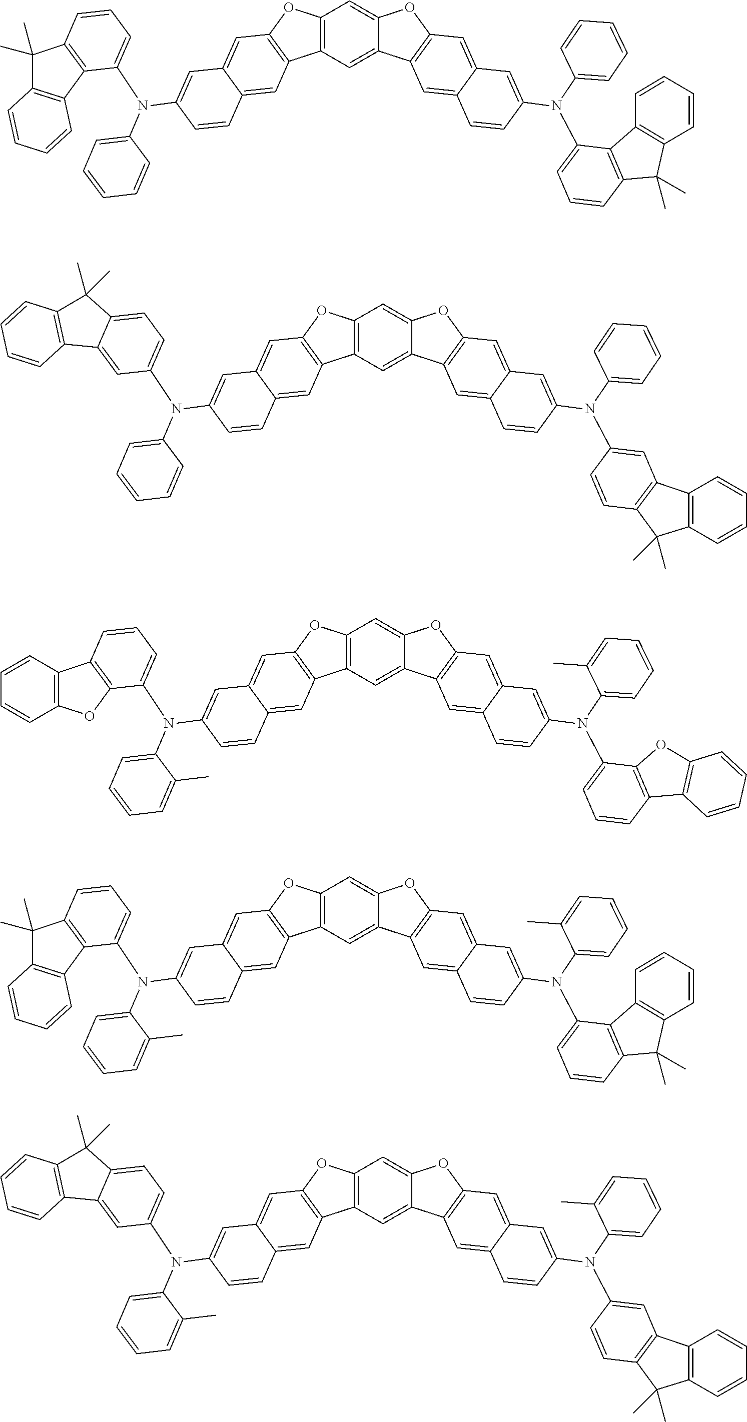

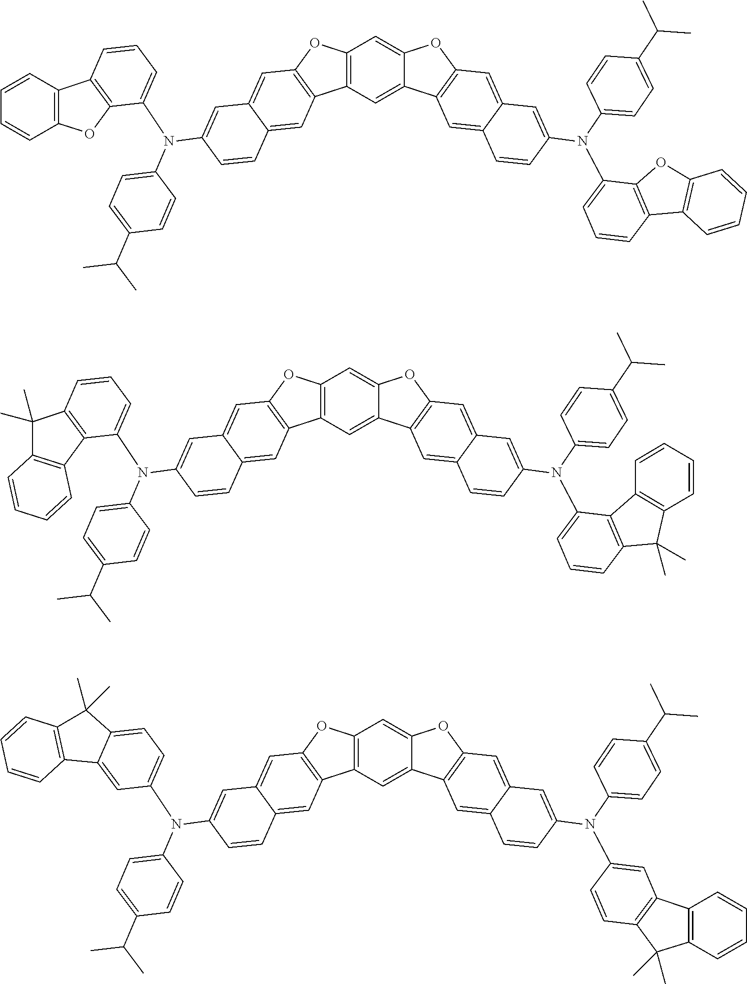 [SMILES: CC(C)c1ccc(N(c2ccc3cc4c(cc3c2)oc2cc3oc5cc6cc(N(c7ccc(C(C)C)cc7)c7cccc8c7oc7ccccc78)ccc6cc5c3cc24)c2cccc3c2oc2ccccc23)cc1 ; CC(C)c1ccc(N(c2ccc3cc4c(cc3c2)oc2cc3oc5cc6cc(N(c7ccc(C(C)C)cc7)c7cccc8c7-c7ccccc7C8(C)C)ccc6cc5c3cc24)c2cccc3c2-c2ccccc2C3(C)C)cc1 ; CC(C)c1ccc(N(c2ccc3c(c2)-c2ccccc2C3(C)C)c2ccc3cc4c(cc3c2)oc2cc3oc5cc6cc(N(c7ccc(C(C)C)cc7)c7ccc8c(c7)-c7ccccc7C8(C)C)ccc6cc5c3cc24)cc1]

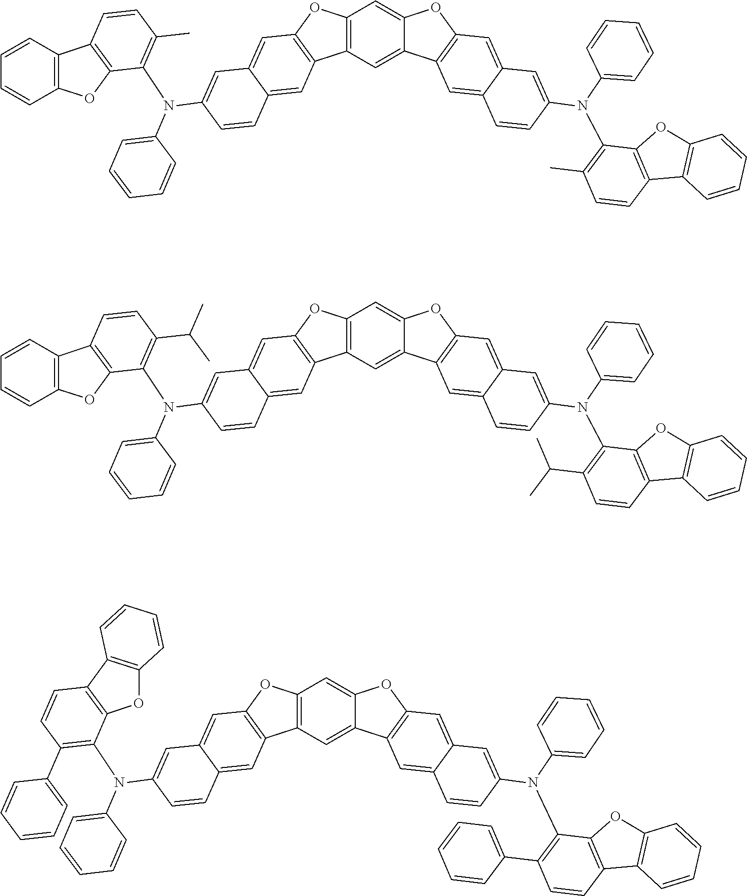 [SMILES: Cc1ccc2c(oc3ccccc32)c1N(c1ccccc1)c1ccc2cc3c(cc2c1)oc1cc2oc4cc5cc(N(c6ccccc6)c6c(C)ccc7c6oc6ccccc67)ccc5cc4c2cc13 ; CC(C)c1ccc2c(oc3ccccc32)c1N(c1ccccc1)c1ccc2cc3c(cc2c1)oc1cc2oc4cc5cc(N(c6ccccc6)c6c(C(C)C)ccc7c6oc6ccccc67)ccc5cc4c2cc13 ; c1ccc(-c2ccc3c(oc4ccccc43)c2N(c2ccccc2)c2ccc3cc4c(cc3c2)oc2cc3oc5cc6cc(N(c7ccccc7)c7c(-c8ccccc8)ccc8c7oc7ccccc78)ccc6cc5c3cc24)cc1]

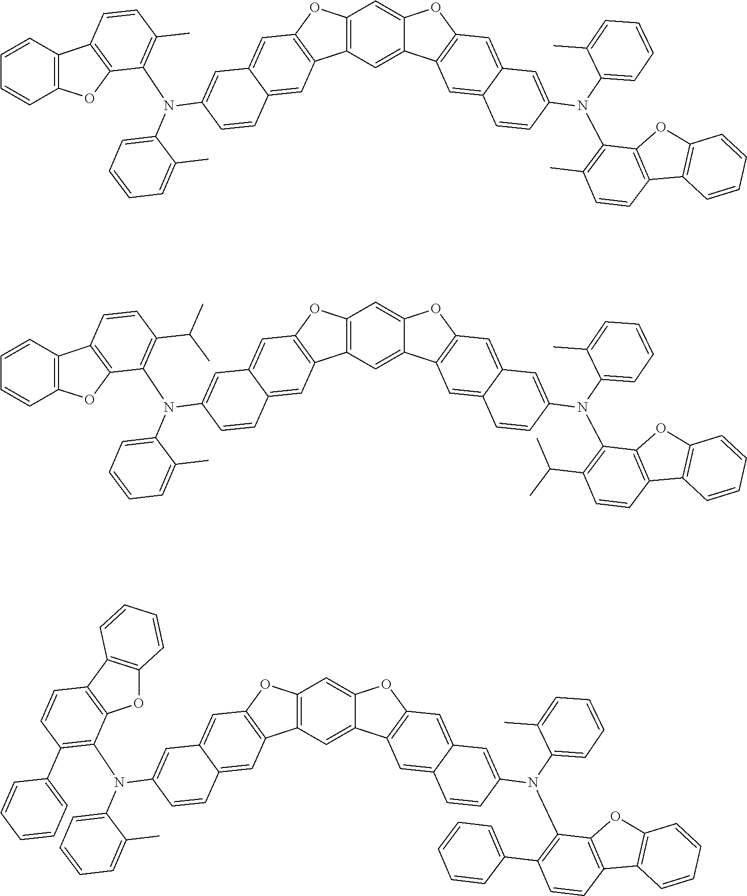 [SMILES: Cc1ccccc1N(c1ccc2cc3c(cc2c1)oc1cc2oc4cc5cc(N(c6ccccc6C)c6c(C)ccc7c6oc6ccccc67)ccc5cc4c2cc13)c1c(C)ccc2c1oc1ccccc12 ; Cc1ccccc1N(c1ccc2cc3c(cc2c1)oc1cc2oc4cc5cc(N(c6ccccc6C)c6c(C(C)C)ccc7c6oc6ccccc67)ccc5cc4c2cc13)c1c(C(C)C)ccc2c1oc1ccccc12 ; Cc1ccccc1N(c1ccc2cc3c(cc2c1)oc1cc2oc4cc5cc(N(c6ccccc6C)c6c(-c7ccccc7)ccc7c6oc6ccccc67)ccc5cc4c2cc13)c1c(-c2ccccc2)ccc2c1oc1ccccc12]

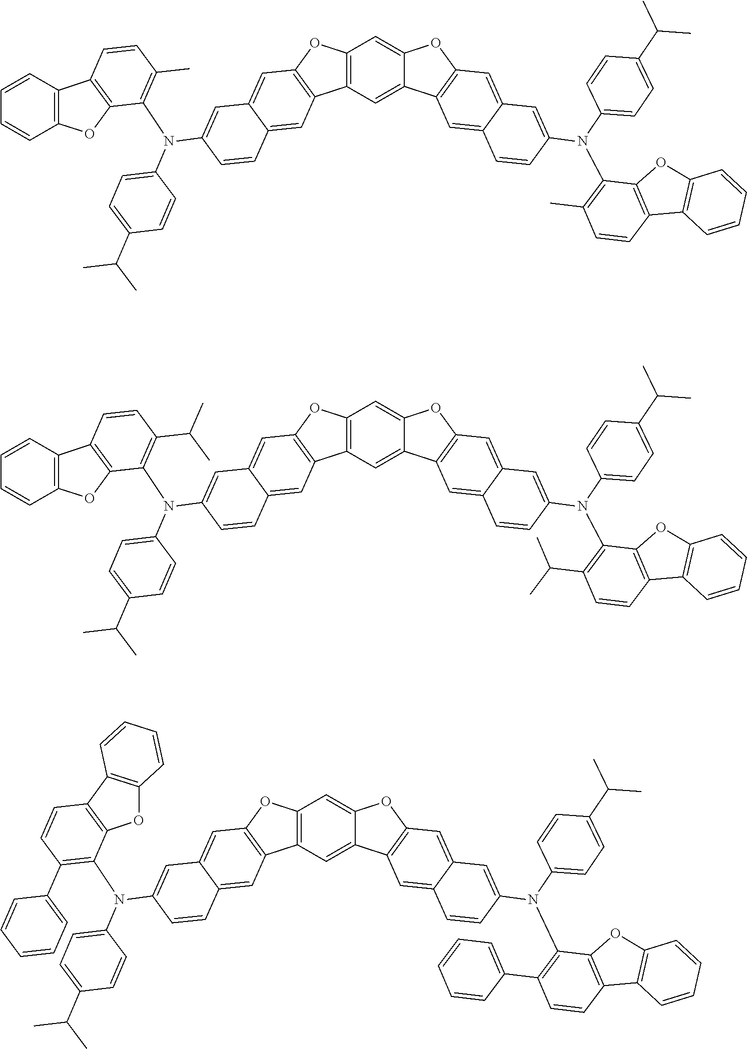 [SMILES: Cc1ccc2c(oc3ccccc32)c1N(c1ccc(C(C)C)cc1)c1ccc2cc3c(cc2c1)oc1cc2oc4cc5cc(N(c6ccc(C(C)C)cc6)c6c(C)ccc7c6oc6ccccc67)ccc5cc4c2cc13 ; CC(C)c1ccc(N(c2ccc3cc4c(cc3c2)oc2cc3oc5cc6cc(N(c7ccc(C(C)C)cc7)c7c(C(C)C)ccc8c7oc7ccccc78)ccc6cc5c3cc24)c2c(C(C)C)ccc3c2oc2ccccc23)cc1 ; CC(C)c1ccc(N(c2ccc3cc4c(cc3c2)oc2cc3oc5cc6cc(N(c7ccc(C(C)C)cc7)c7c(-c8ccccc8)ccc8c7oc7ccccc78)ccc6cc5c3cc24)c2c(-c3ccccc3)ccc3c2oc2ccccc23)cc1]

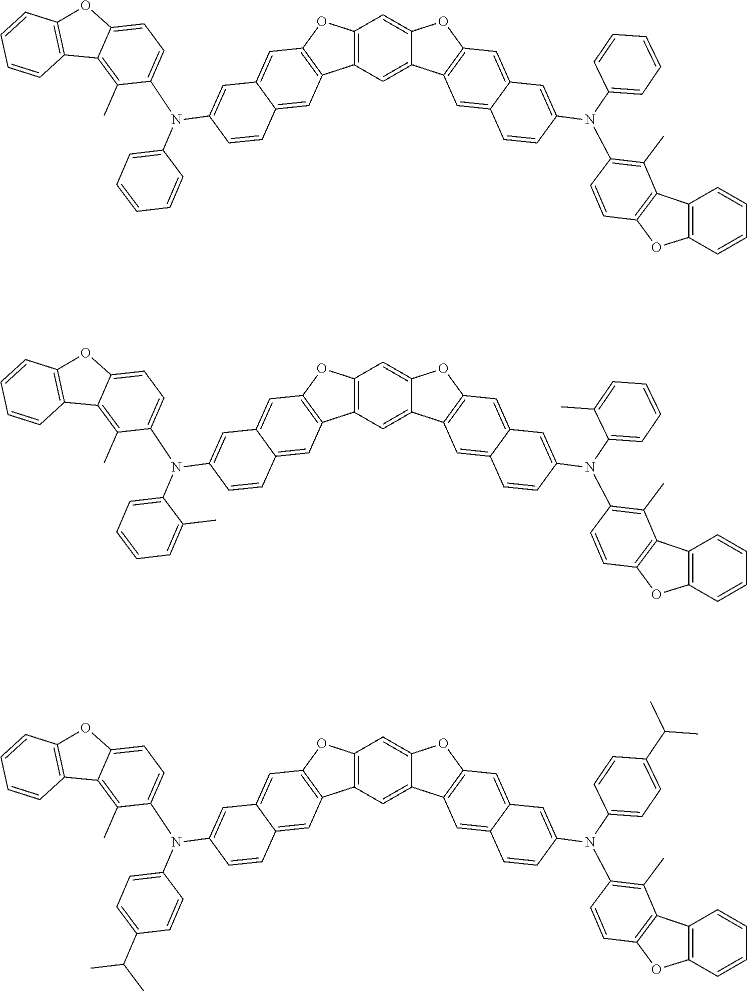 [SMILES: Cc1c(N(c2ccccc2)c2ccc3cc4c(cc3c2)oc2cc3oc5cc6cc(N(c7ccccc7)c7ccc8oc9ccccc9c8c7C)ccc6cc5c3cc24)ccc2oc3ccccc3c12 ; Cc1ccccc1N(c1ccc2cc3c(cc2c1)oc1cc2oc4cc5cc(N(c6ccccc6C)c6ccc7oc8ccccc8c7c6C)ccc5cc4c2cc13)c1ccc2oc3ccccc3c2c1C ; Cc1c(N(c2ccc(C(C)C)cc2)c2ccc3cc4c(cc3c2)oc2cc3oc5cc6cc(N(c7ccc(C(C)C)cc7)c7ccc8oc9ccccc9c8c7C)ccc6cc5c3cc24)ccc2oc3ccccc3c12]

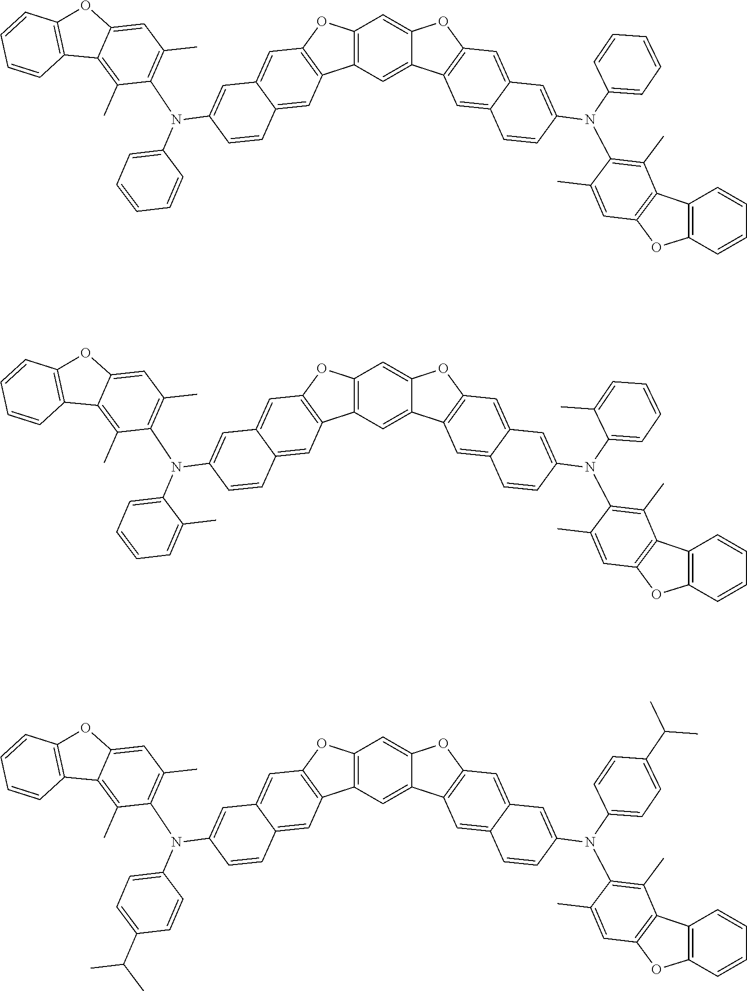 [SMILES: Cc1cc2oc3ccccc3c2c(C)c1N(c1ccccc1)c1ccc2cc3c(cc2c1)oc1cc2oc4cc5cc(N(c6ccccc6)c6c(C)cc7oc8ccccc8c7c6C)ccc5cc4c2cc13 ; Cc1ccccc1N(c1ccc2cc3c(cc2c1)oc1cc2oc4cc5cc(N(c6ccccc6C)c6c(C)cc7oc8ccccc8c7c6C)ccc5cc4c2cc13)c1c(C)cc2oc3ccccc3c2c1C ; Cc1cc2oc3ccccc3c2c(C)c1N(c1ccc(C(C)C)cc1)c1ccc2cc3c(cc2c1)oc1cc2oc4cc5cc(N(c6ccc(C(C)C)cc6)c6c(C)cc7oc8ccccc8c7c6C)ccc5cc4c2cc13]

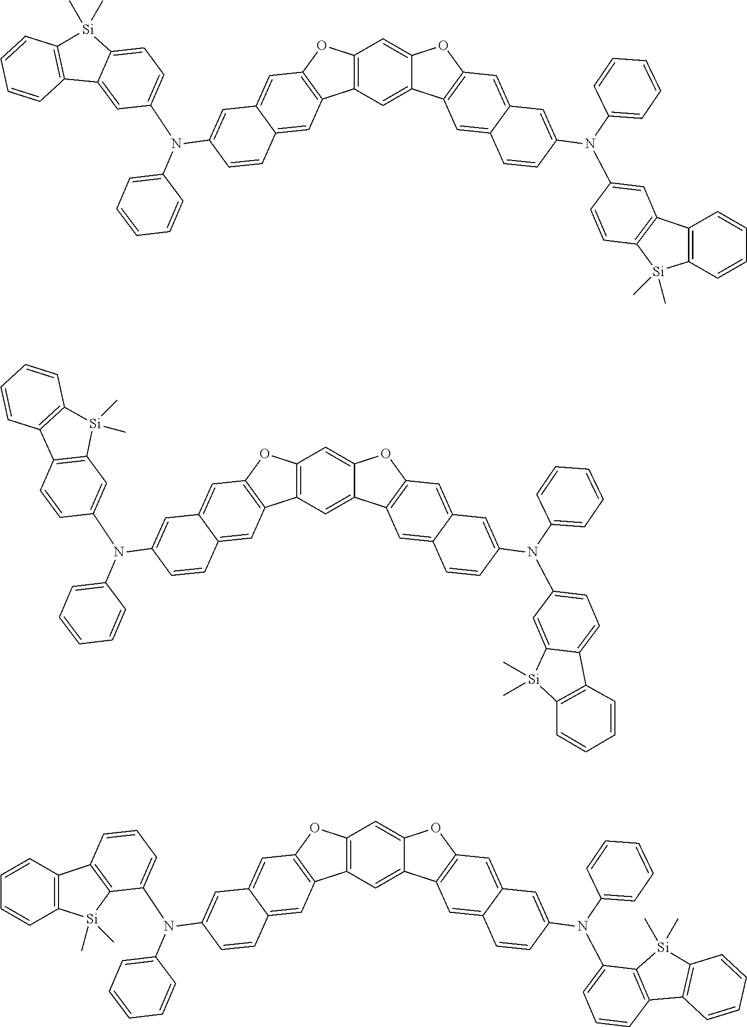 [SMILES: C[Si]1(C)c2ccccc2-c2cc(N(c3ccccc3)c3ccc4cc5c(cc4c3)oc3cc4oc6cc7cc(N(c8ccccc8)c8ccc9c(c8)-c8ccccc8[Si]9(C)C)ccc7cc6c4cc35)ccc21 ; C[Si]1(C)c2ccccc2-c2ccc(N(c3ccccc3)c3ccc4cc5c(cc4c3)oc3cc4oc6cc7cc(N(c8ccccc8)c8ccc9c(c8)[Si](C)(C)c8ccccc8-9)ccc7cc6c4cc35)cc21 ; C[Si]1(C)c2ccccc2-c2cccc(N(c3ccccc3)c3ccc4cc5c(cc4c3)oc3cc4oc6cc7cc(N(c8ccccc8)c8cccc9c8[Si](C)(C)c8ccccc8-9)ccc7cc6c4cc35)c21]

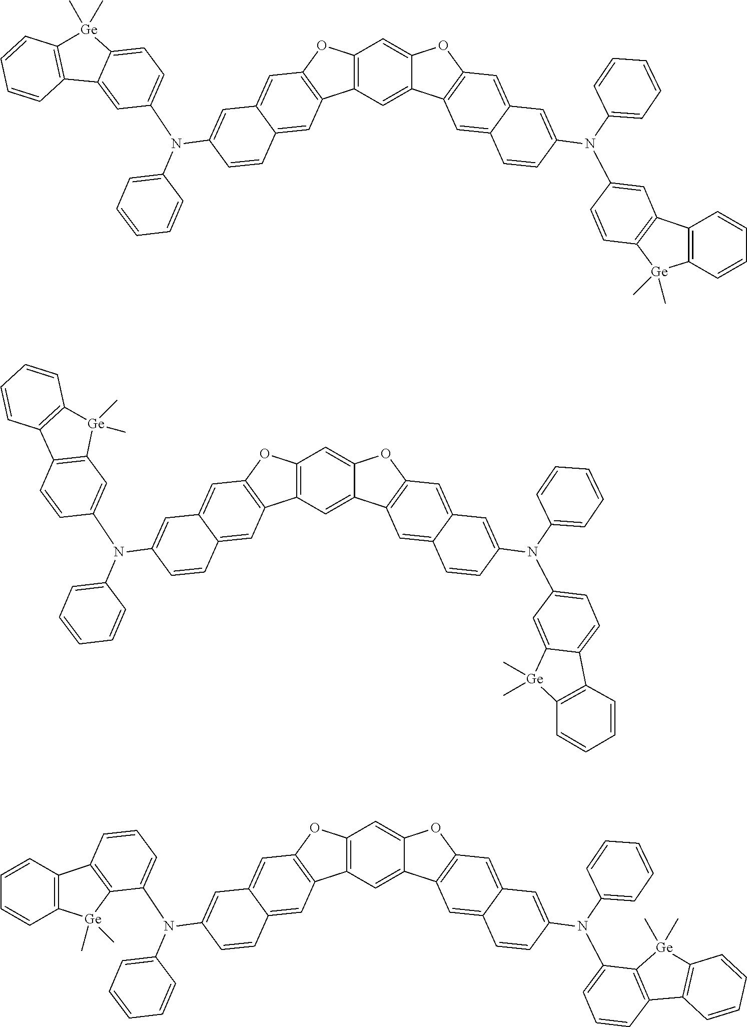

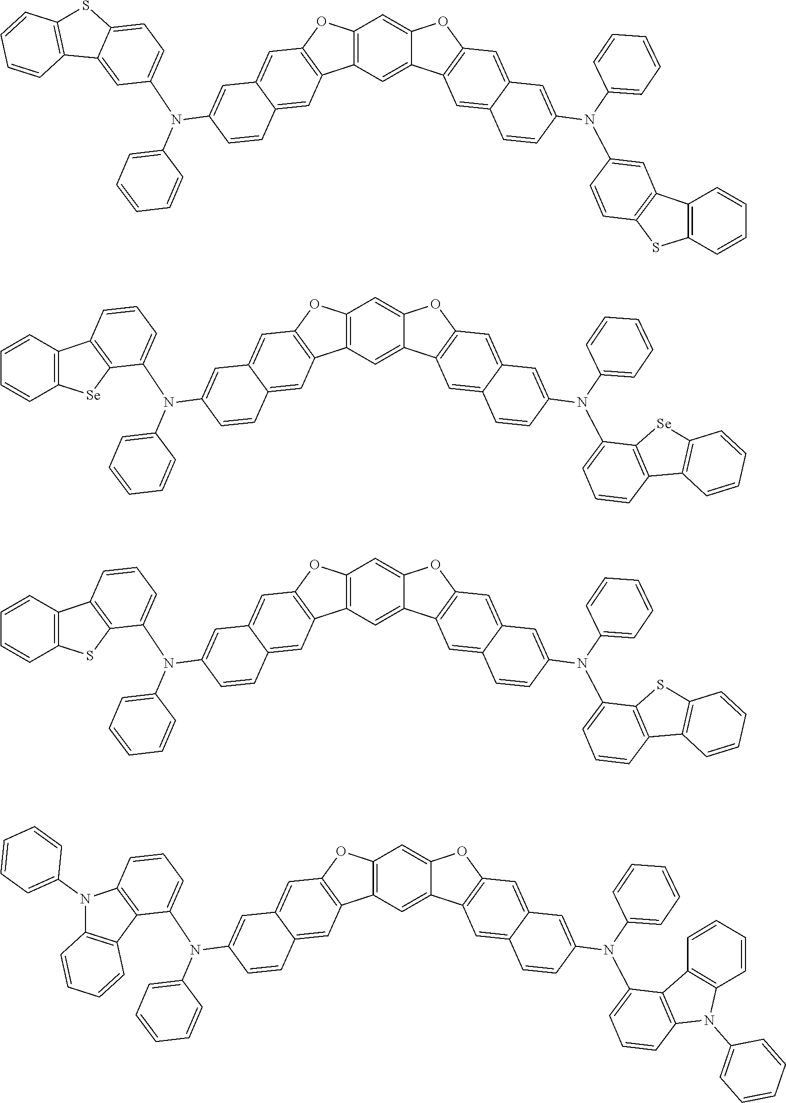 [SMILES: c1ccc(N(c2ccc3cc4c(cc3c2)oc2cc3oc5cc6cc(N(c7ccccc7)c7ccc8sc9ccccc9c8c7)ccc6cc5c3cc24)c2ccc3sc4ccccc4c3c2)cc1 ; c1ccc(N(c2ccc3cc4c(cc3c2)oc2cc3oc5cc6cc(N(c7ccccc7)c7cccc8c7[se]c7ccccc78)ccc6cc5c3cc24)c2cccc3c2[se]c2ccccc23)cc1 ; c1ccc(N(c2ccc3cc4c(cc3c2)oc2cc3oc5cc6cc(N(c7ccccc7)c7cccc8c7sc7ccccc78)ccc6cc5c3cc24)c2cccc3c2sc2ccccc23)cc1 ; c1ccc(N(c2ccc3cc4c(cc3c2)oc2cc3oc5cc6cc(N(c7ccccc7)c7cccc8c7c7ccccc7n8-c7ccccc7)ccc6cc5c3cc24)c2cccc3c2c2ccccc2n3-c2ccccc2)cc1]

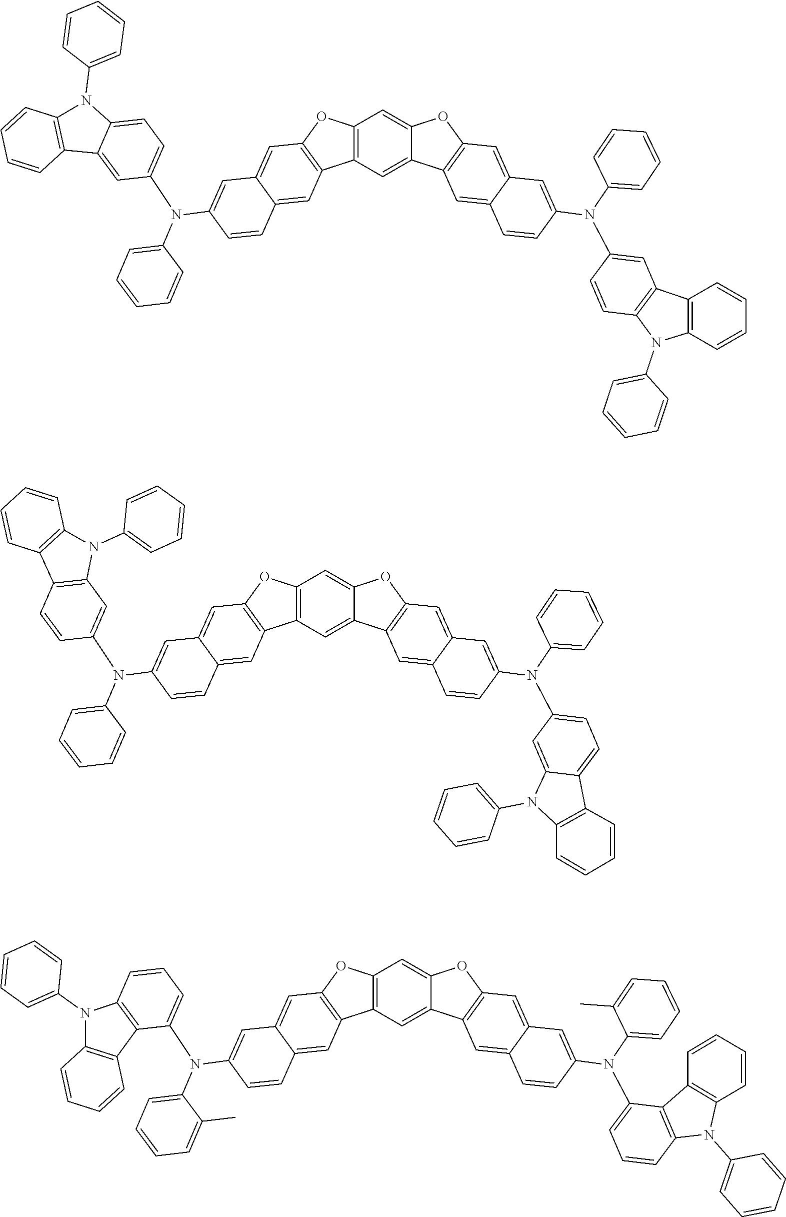 [SMILES: c1ccc(N(c2ccc3cc4c(cc3c2)oc2cc3oc5cc6cc(N(c7ccccc7)c7ccc8c(c7)c7ccccc7n8-c7ccccc7)ccc6cc5c3cc24)c2ccc3c(c2)c2ccccc2n3-c2ccccc2)cc1 ; c1ccc(N(c2ccc3cc4c(cc3c2)oc2cc3oc5cc6cc(N(c7ccccc7)c7ccc8c9ccccc9n(-c9ccccc9)c8c7)ccc6cc5c3cc24)c2ccc3c4ccccc4n(-c4ccccc4)c3c2)cc1 ; Cc1ccccc1N(c1ccc2cc3c(cc2c1)oc1cc2oc4cc5cc(N(c6ccccc6C)c6cccc7c6c6ccccc6n7-c6ccccc6)ccc5cc4c2cc13)c1cccc2c1c1ccccc1n2-c1ccccc1]

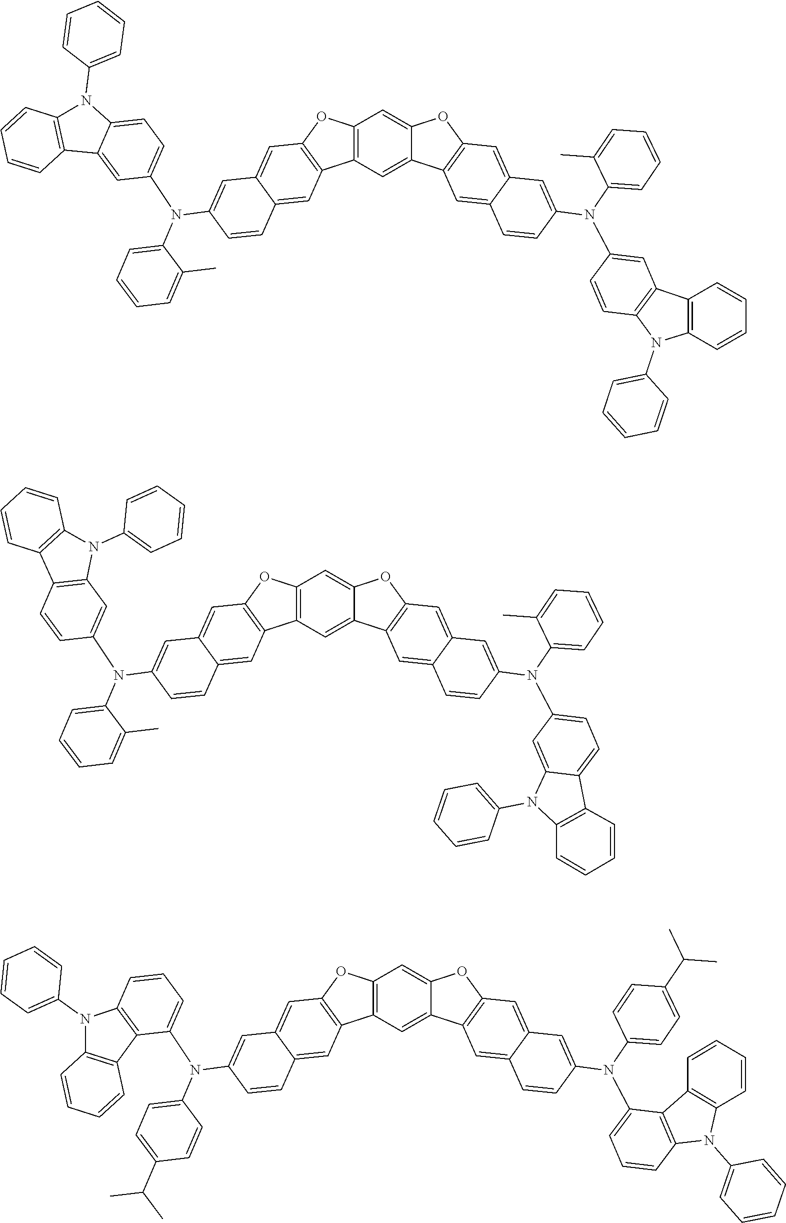 [SMILES: Cc1ccccc1N(c1ccc2cc3c(cc2c1)oc1cc2oc4cc5cc(N(c6ccc7c(c6)c6ccccc6n7-c6ccccc6)c6ccccc6C)ccc5cc4c2cc13)c1ccc2c(c1)c1ccccc1n2-c1ccccc1 ; Cc1ccccc1N(c1ccc2cc3c(cc2c1)oc1cc2oc4cc5cc(N(c6ccc7c8ccccc8n(-c8ccccc8)c7c6)c6ccccc6C)ccc5cc4c2cc13)c1ccc2c3ccccc3n(-c3ccccc3)c2c1 ; CC(C)c1ccc(N(c2ccc3cc4c(cc3c2)oc2cc3oc5cc6cc(N(c7ccc(C(C)C)cc7)c7cccc8c7c7ccccc7n8-c7ccccc7)ccc6cc5c3cc24)c2cccc3c2c2ccccc2n3-c2ccccc2)cc1]

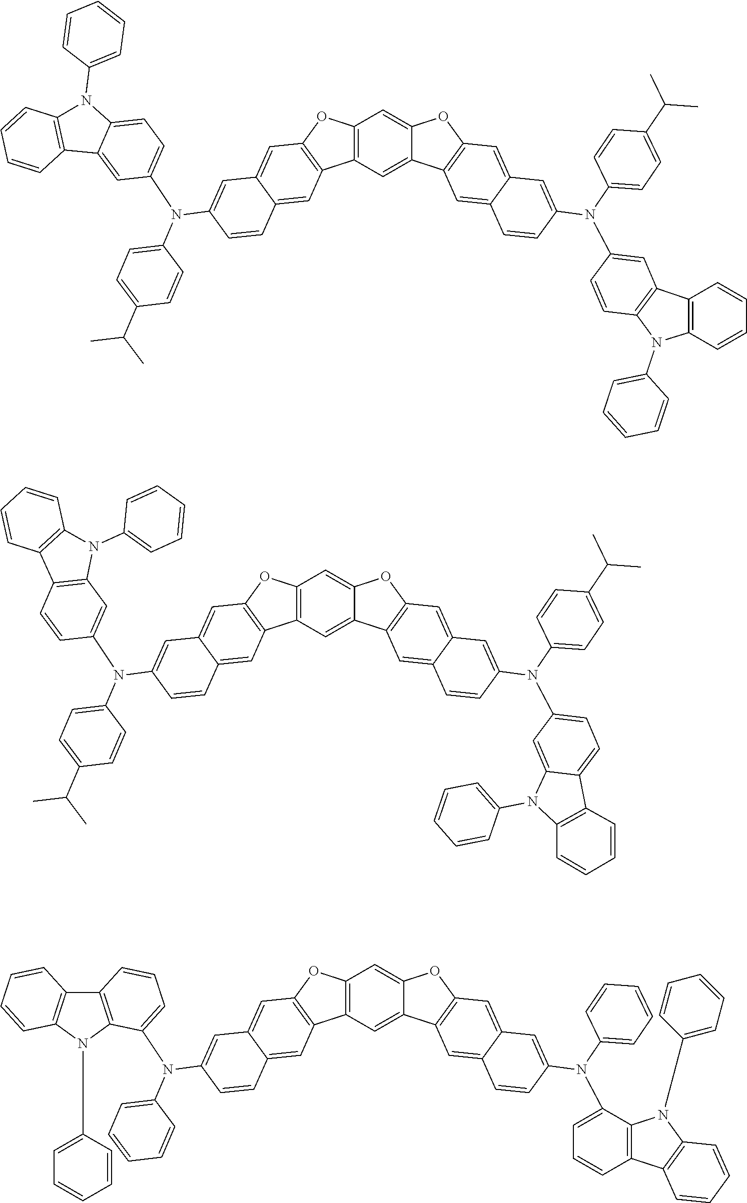 [SMILES: CC(C)c1ccc(N(c2ccc3cc4c(cc3c2)oc2cc3oc5cc6cc(N(c7ccc(C(C)C)cc7)c7ccc8c(c7)c7ccccc7n8-c7ccccc7)ccc6cc5c3cc24)c2ccc3c(c2)c2ccccc2n3-c2ccccc2)cc1 ; CC(C)c1ccc(N(c2ccc3cc4c(cc3c2)oc2cc3oc5cc6cc(N(c7ccc(C(C)C)cc7)c7ccc8c9ccccc9n(-c9ccccc9)c8c7)ccc6cc5c3cc24)c2ccc3c4ccccc4n(-c4ccccc4)c3c2)cc1 ; c1ccc(N(c2ccc3cc4c(cc3c2)oc2cc3oc5cc6cc(N(c7ccccc7)c7cccc8c9ccccc9n(-c9ccccc9)c78)ccc6cc5c3cc24)c2cccc3c4ccccc4n(-c4ccccc4)c23)cc1]

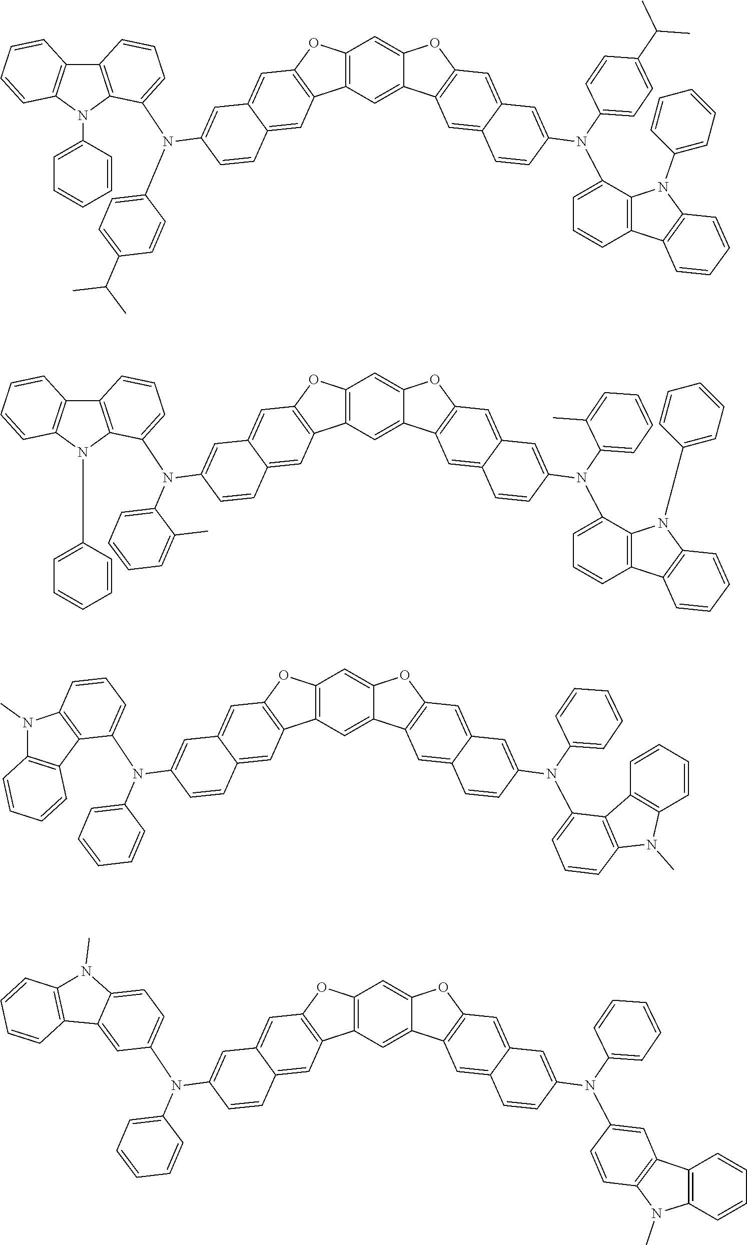

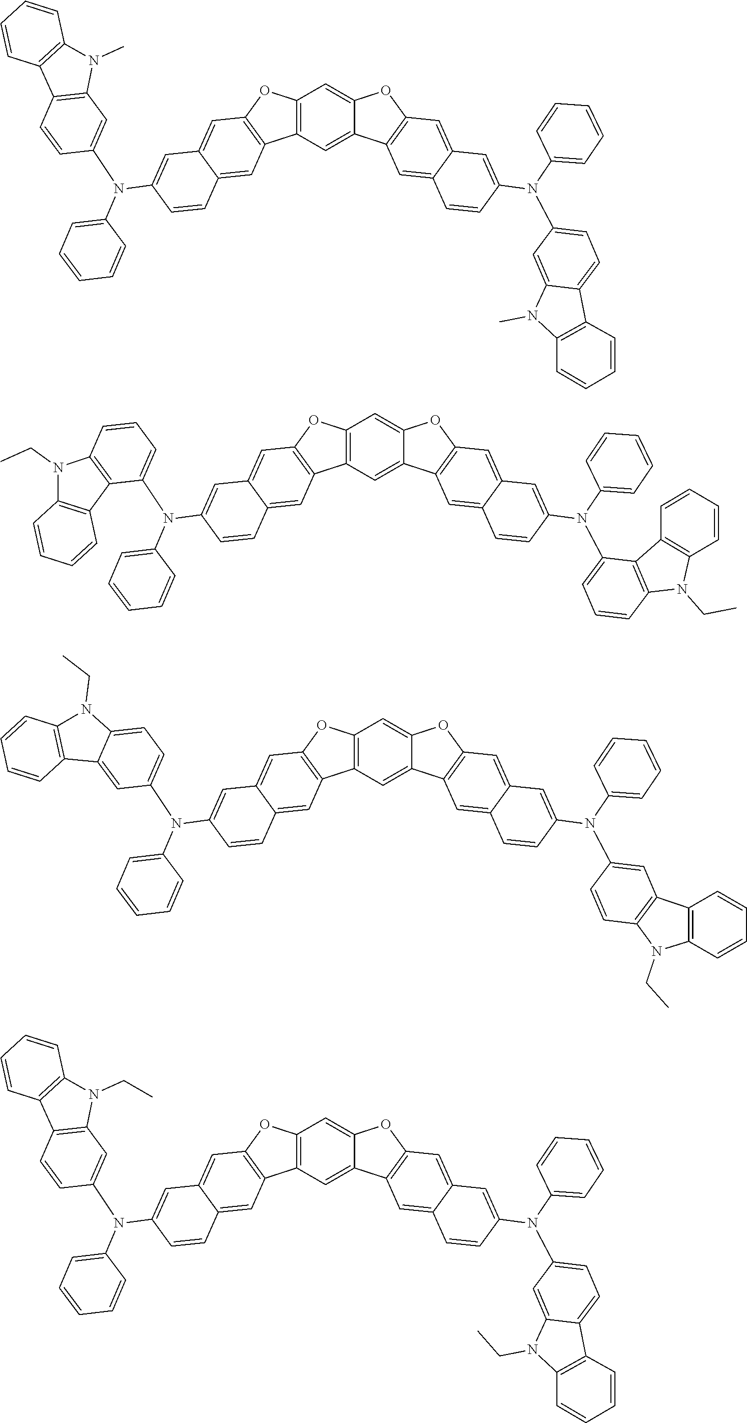 [SMILES: Cn1c2ccccc2c2ccc(N(c3ccccc3)c3ccc4cc5c(cc4c3)oc3cc4oc6cc7cc(N(c8ccccc8)c8ccc9c%10ccccc%10n(C)c9c8)ccc7cc6c4cc35)cc21 ; CCn1c2ccccc2c2c(N(c3ccccc3)c3ccc4cc5c(cc4c3)oc3cc4oc6cc7cc(N(c8ccccc8)c8cccc9c8c8ccccc8n9CC)ccc7cc6c4cc35)cccc21 ; CCn1c2ccccc2c2cc(N(c3ccccc3)c3ccc4cc5c(cc4c3)oc3cc4oc6cc7cc(N(c8ccccc8)c8ccc9c(c8)c8ccccc8n9CC)ccc7cc6c4cc35)ccc21 ; CCn1c2ccccc2c2ccc(N(c3ccccc3)c3ccc4cc5c(cc4c3)oc3cc4oc6cc7cc(N(c8ccccc8)c8ccc9c%10ccccc%10n(CC)c9c8)ccc7cc6c4cc35)cc21]

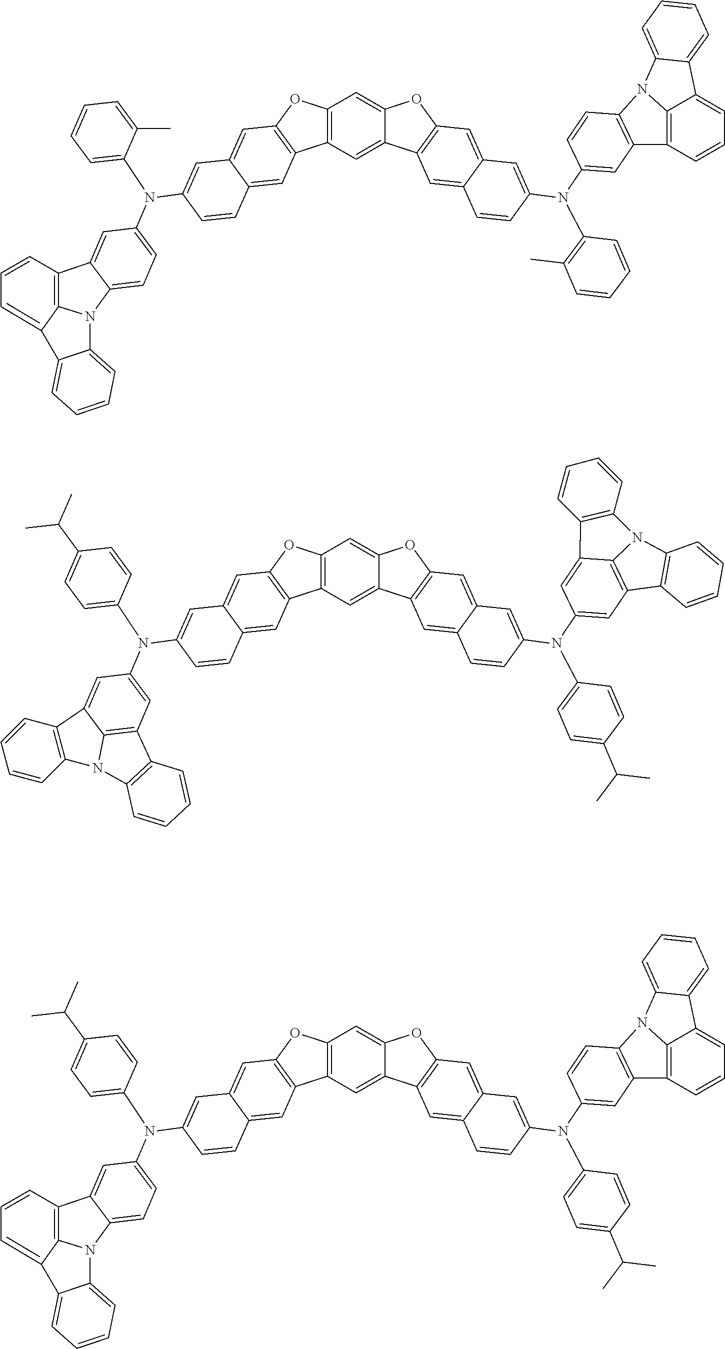 [SMILES: Cc1ccccc1N(c1ccc2cc3c(cc2c1)oc1cc2oc4cc5cc(N(c6ccc7c(c6)c6cccc8c9ccccc9n7c86)c6ccccc6C)ccc5cc4c2cc13)c1ccc2c(c1)c1cccc3c4ccccc4n2c31 ; CC(C)c1ccc(N(c2ccc3cc4c(cc3c2)oc2cc3oc5cc6cc(N(c7ccc(C(C)C)cc7)c7cc8c9ccccc9n9c%10ccccc%10c(c7)c89)ccc6cc5c3cc24)c2cc3c4ccccc4n4c5ccccc5c(c2)c34)cc1 ; CC(C)c1ccc(N(c2ccc3cc4c(cc3c2)oc2cc3oc5cc6cc(N(c7ccc(C(C)C)cc7)c7ccc8c(c7)c7cccc9c%10ccccc%10n8c97)ccc6cc5c3cc24)c2ccc3c(c2)c2cccc4c5ccccc5n3c42)cc1]

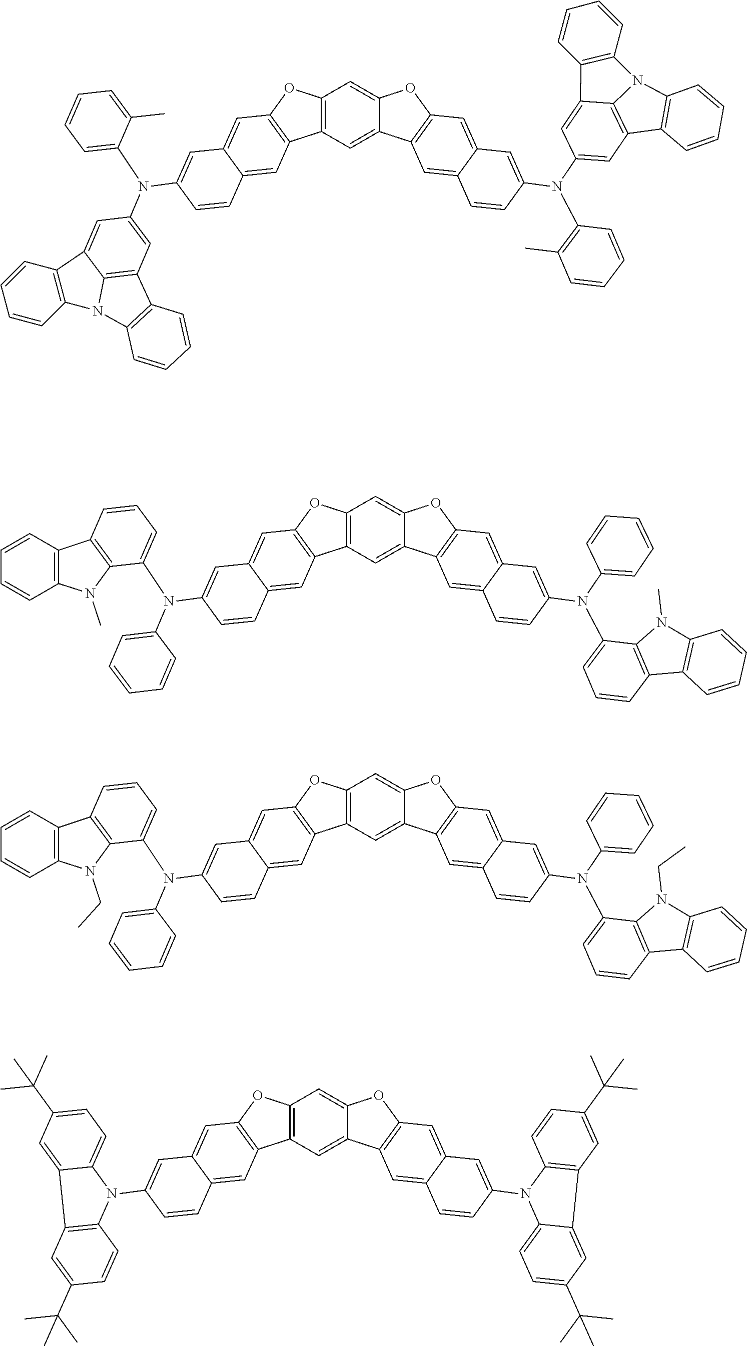

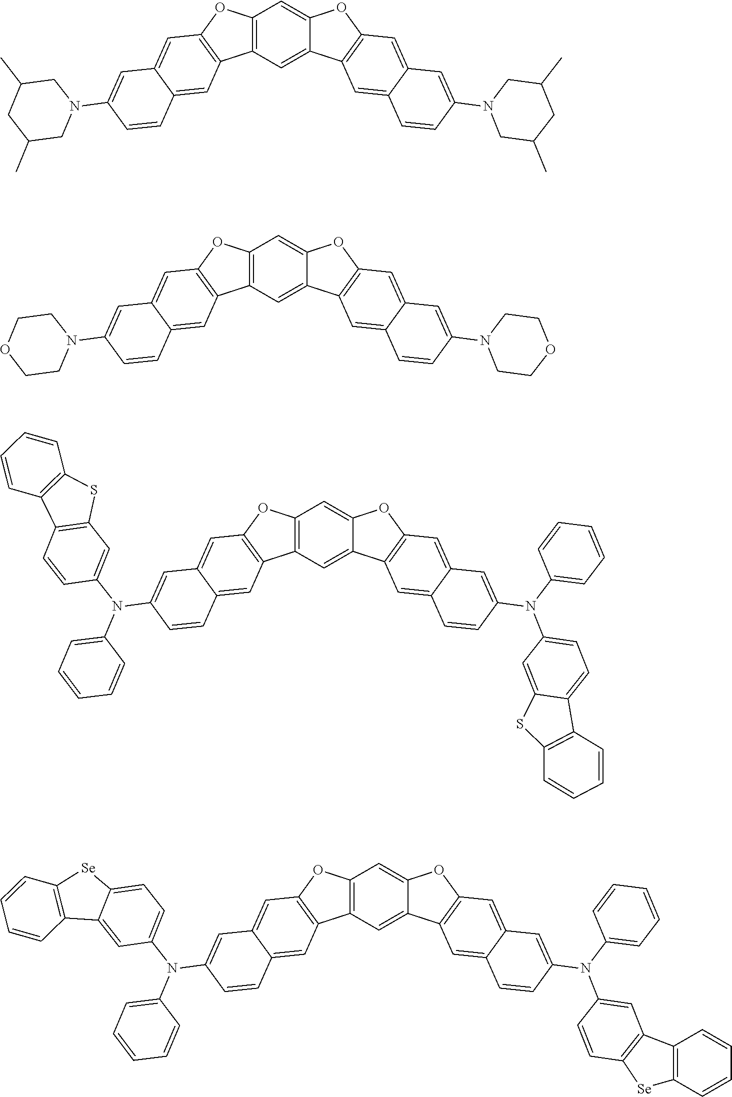

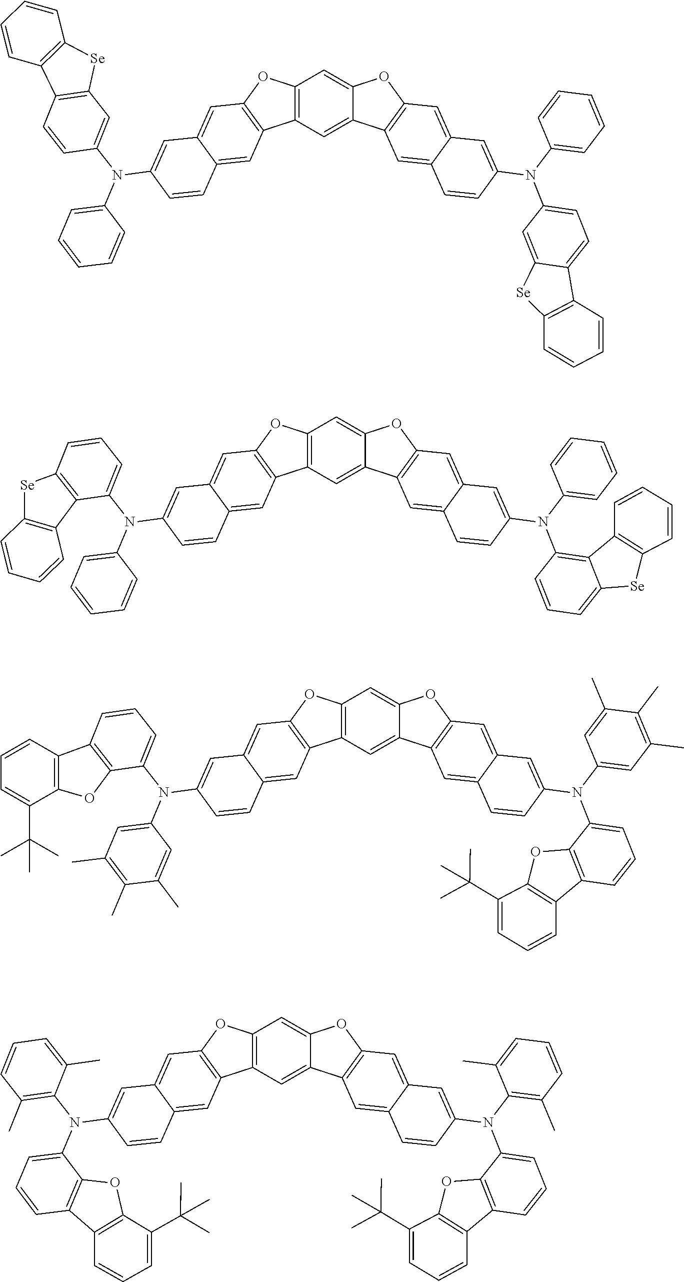

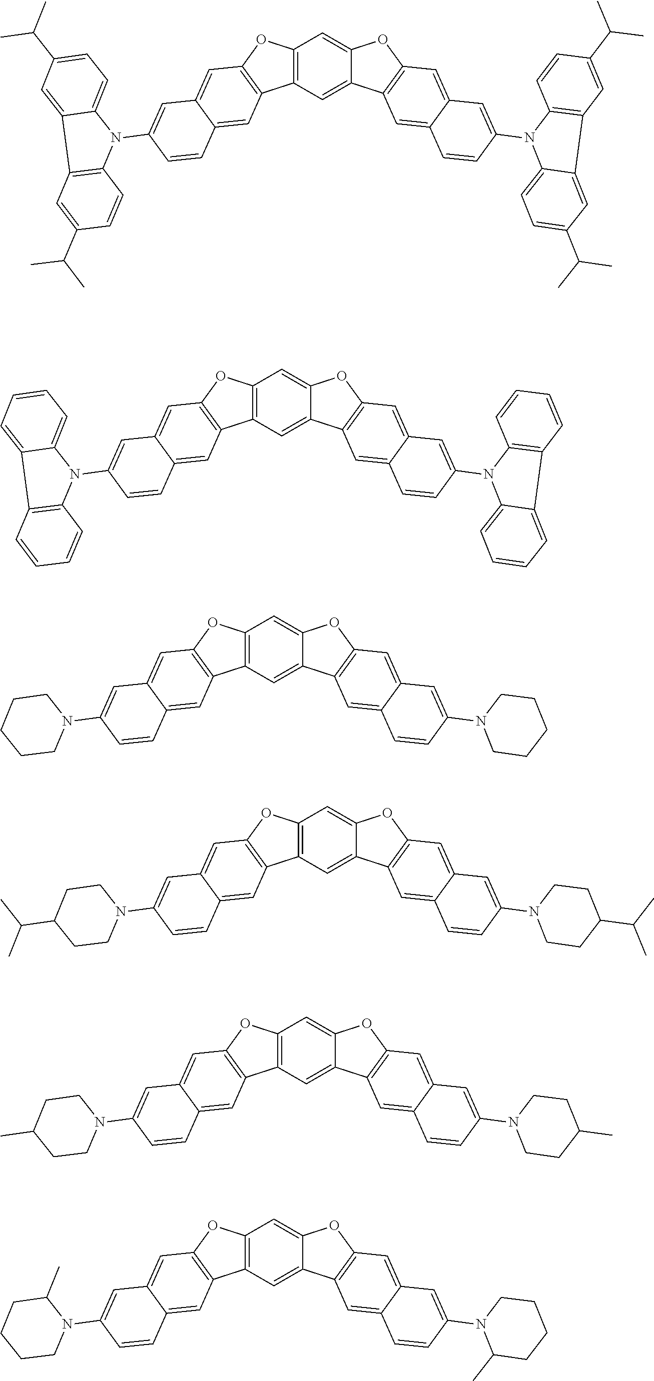

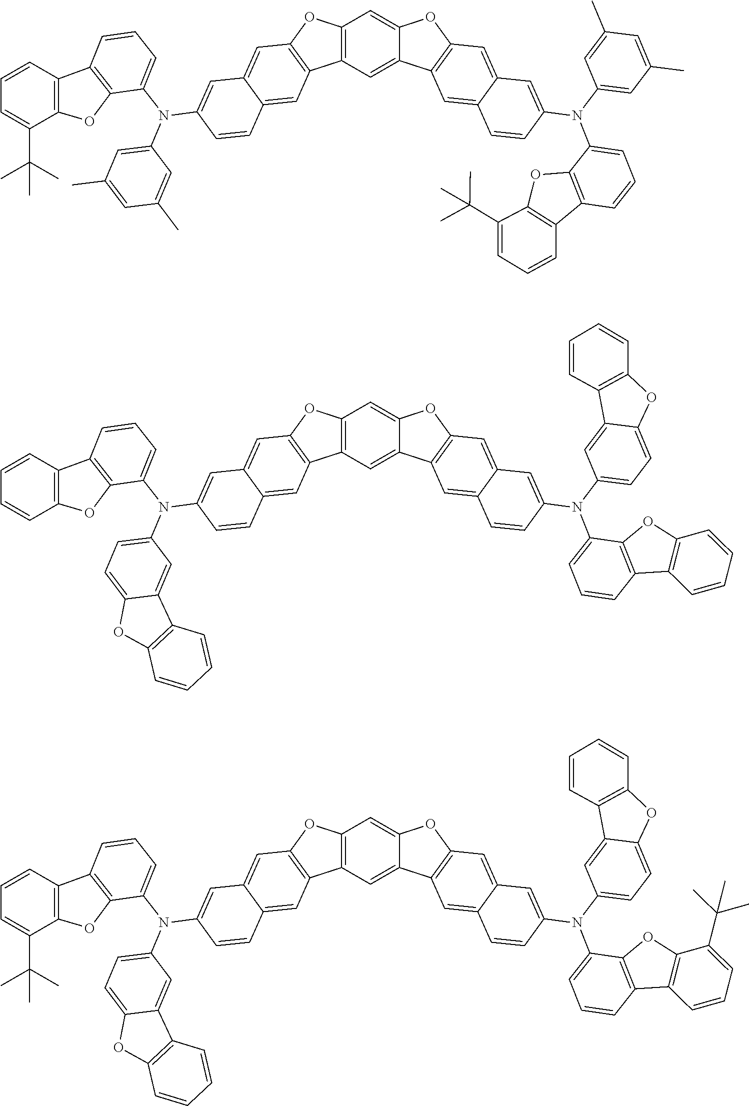

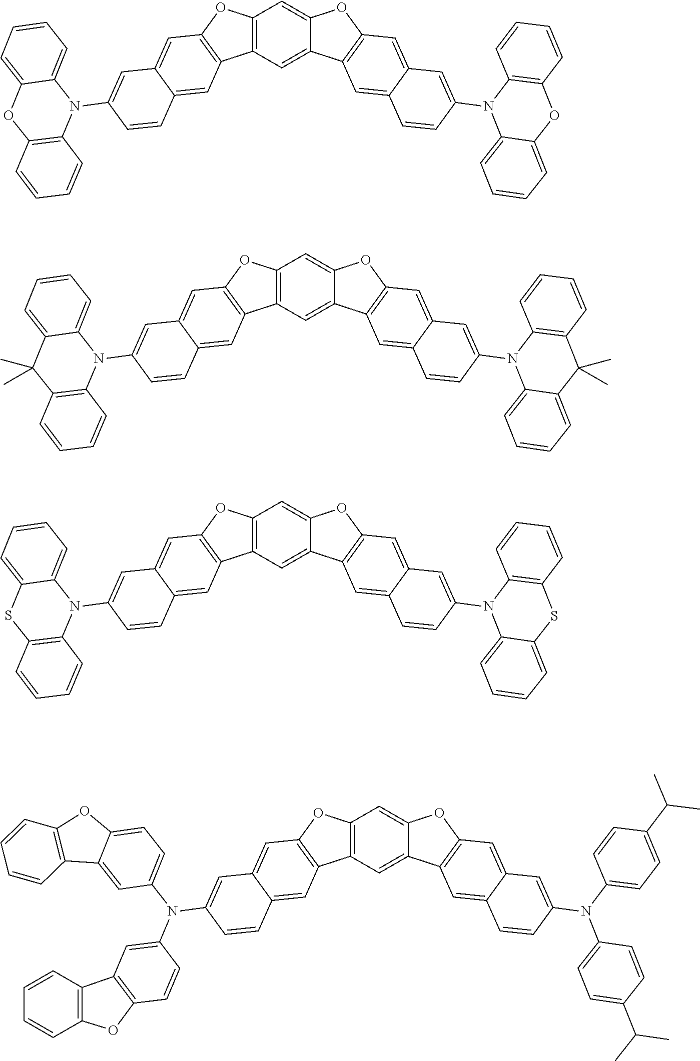

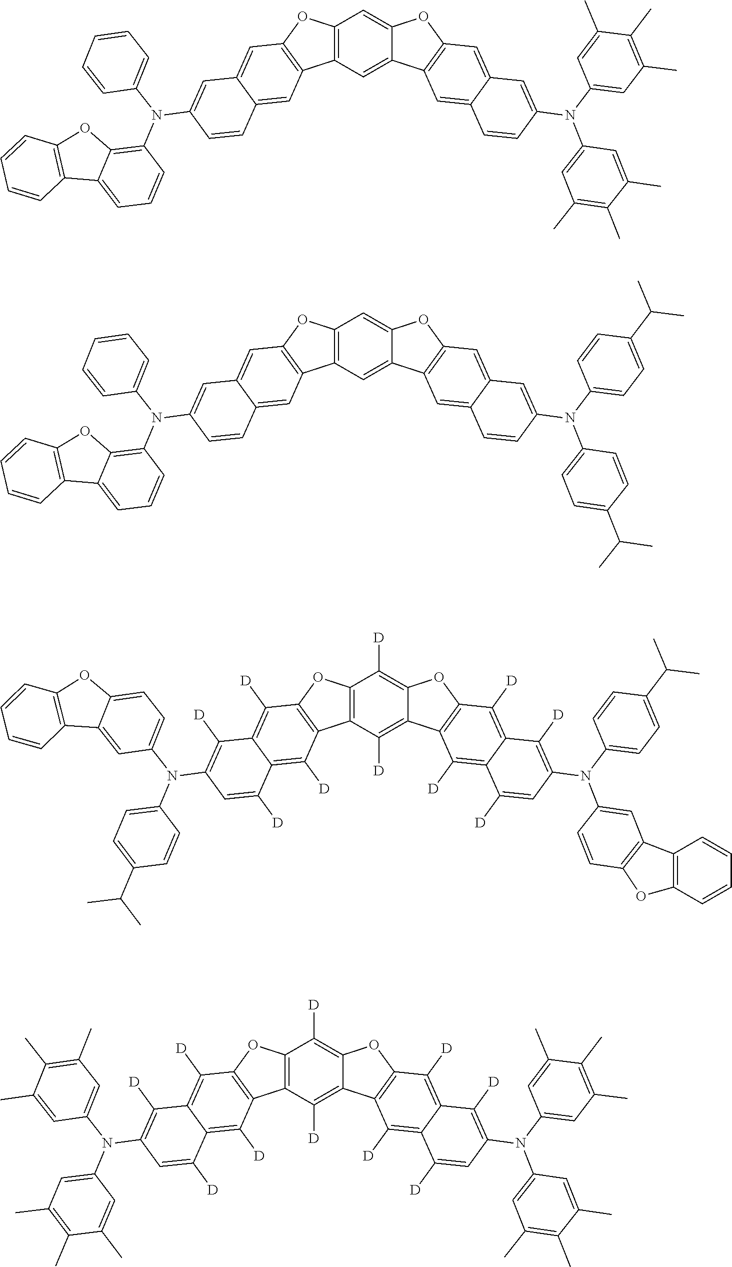

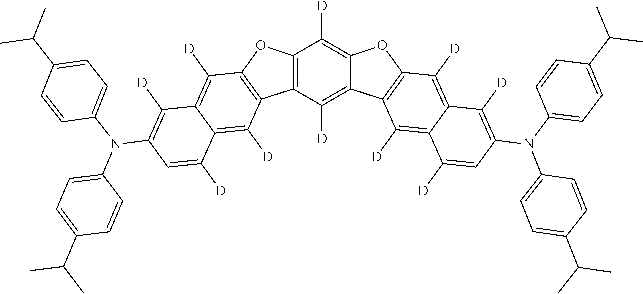 [SMILES: [2H]c1c2oc3c([2H])c4c([2H])c(N(c5ccc(C(C)C)cc5)c5ccc(C(C)C)cc5)cc([2H])c4c([2H])c3c2c([2H])c2c1oc1c([2H])c3c([2H])c(N(c4ccc(C(C)C)cc4)c4ccc(C(C)C)cc4)cc([2H])c3c([2H])c12]

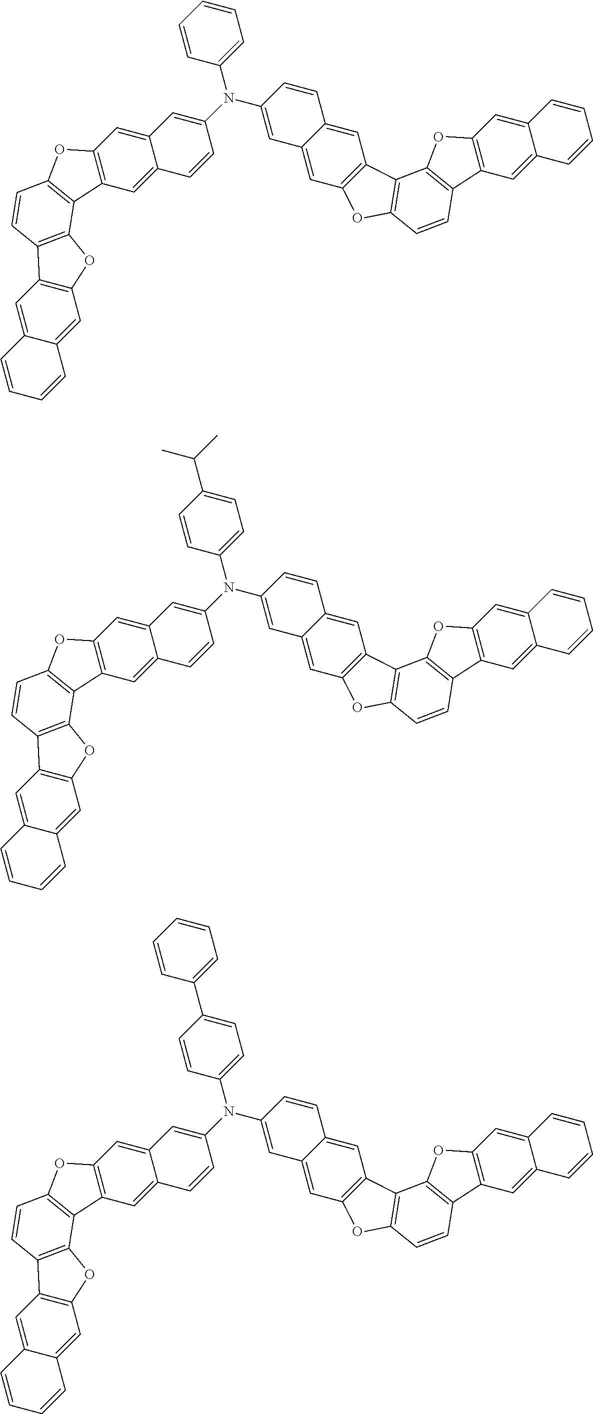 [SMILES: c1ccc(N(c2ccc3cc4c(cc3c2)oc2ccc3c5cc6ccccc6cc5oc3c24)c2ccc3cc4c(cc3c2)oc2ccc3c5cc6ccccc6cc5oc3c24)cc1 ; CC(C)c1ccc(N(c2ccc3cc4c(cc3c2)oc2ccc3c5cc6ccccc6cc5oc3c24)c2ccc3cc4c(cc3c2)oc2ccc3c5cc6ccccc6cc5oc3c24)cc1 ; c1ccc(-c2ccc(N(c3ccc4cc5c(cc4c3)oc3ccc4c6cc7ccccc7cc6oc4c35)c3ccc4cc5c(cc4c3)oc3ccc4c6cc7ccccc7cc6oc4c35)cc2)cc1]

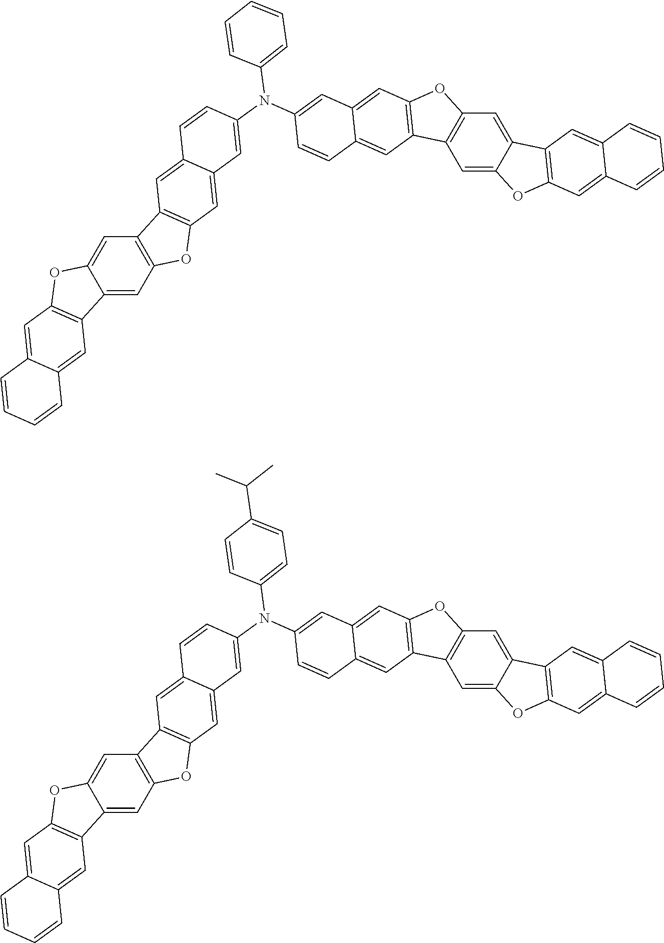 [SMILES: c1ccc(N(c2ccc3cc4c(cc3c2)oc2cc3c(cc24)oc2cc4ccccc4cc23)c2ccc3cc4c(cc3c2)oc2cc3c(cc24)oc2cc4ccccc4cc23)cc1 ; CC(C)c1ccc(N(c2ccc3cc4c(cc3c2)oc2cc3c(cc24)oc2cc4ccccc4cc23)c2ccc3cc4c(cc3c2)oc2cc3c(cc24)oc2cc4ccccc4cc23)cc1]

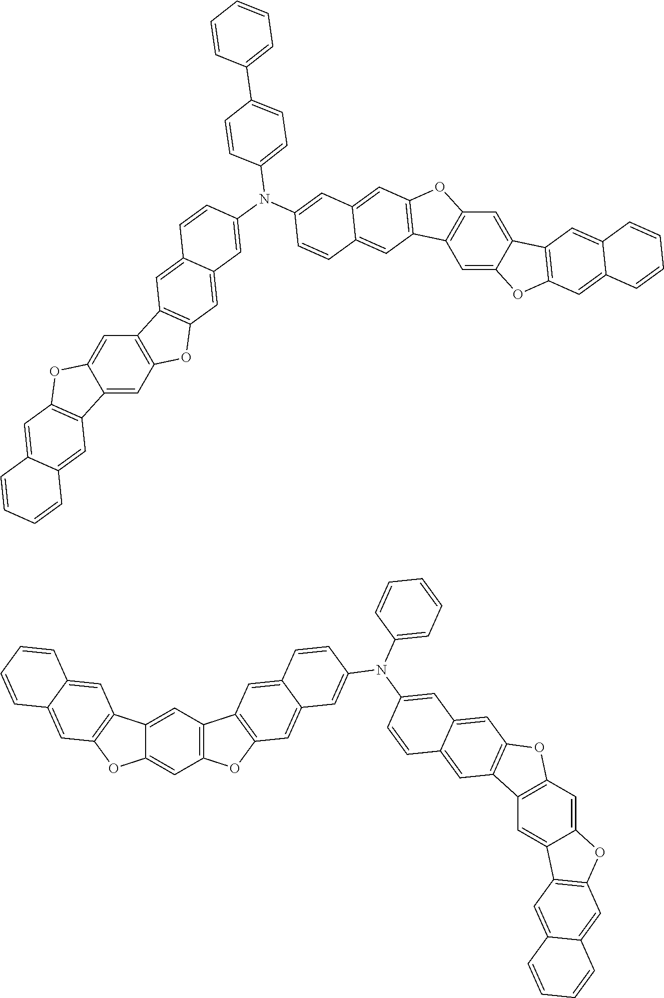 [SMILES: c1ccc(-c2ccc(N(c3ccc4cc5c(cc4c3)oc3cc4c(cc35)oc3cc5ccccc5cc34)c3ccc4cc5c(cc4c3)oc3cc4c(cc35)oc3cc5ccccc5cc34)cc2)cc1 ; c1ccc(N(c2ccc3cc4c(cc3c2)oc2cc3oc5cc6ccccc6cc5c3cc24)c2ccc3cc4c(cc3c2)oc2cc3oc5cc6ccccc6cc5c3cc24)cc1]

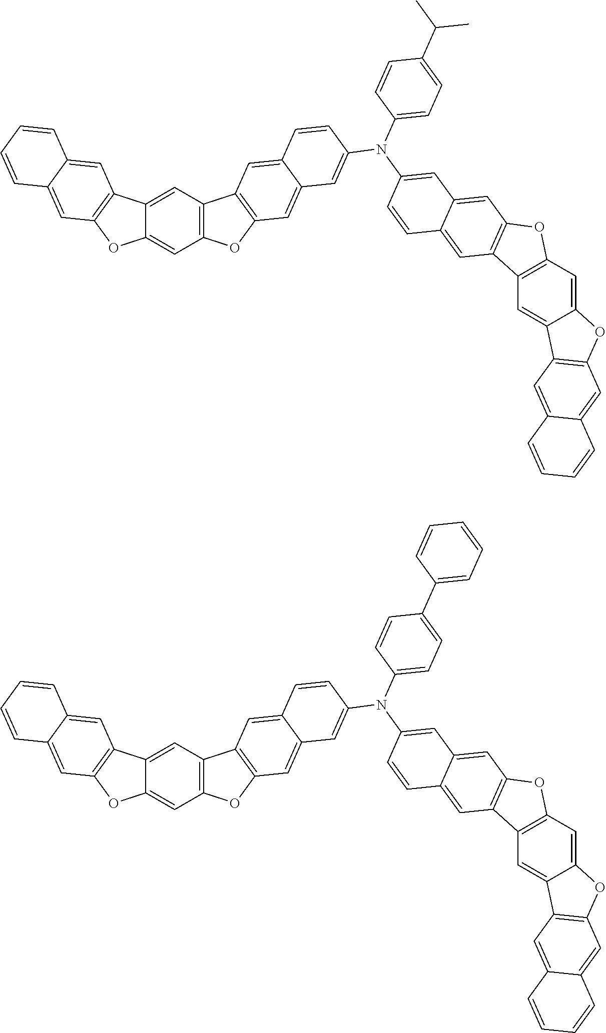 [SMILES: CC(C)c1ccc(N(c2ccc3cc4c(cc3c2)oc2cc3oc5cc6ccccc6cc5c3cc24)c2ccc3cc4c(cc3c2)oc2cc3oc5cc6ccccc6cc5c3cc24)cc1 ; c1ccc(-c2ccc(N(c3ccc4cc5c(cc4c3)oc3cc4oc6cc7ccccc7cc6c4cc35)c3ccc4cc5c(cc4c3)oc3cc4oc6cc7ccccc7cc6c4cc35)cc2)cc1]

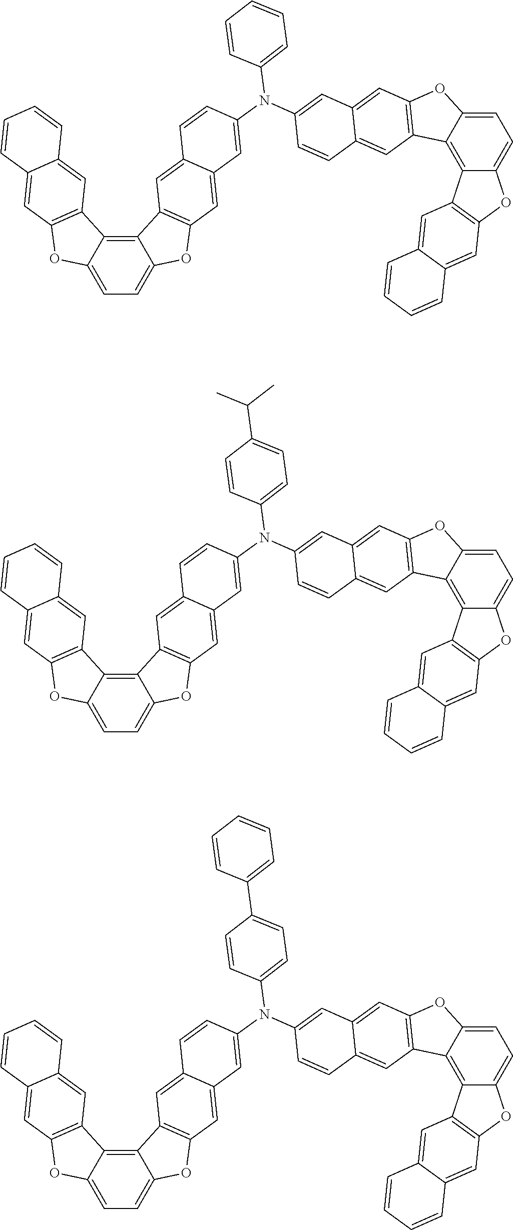 [SMILES: c1ccc(N(c2ccc3cc4c(cc3c2)oc2ccc3oc5cc6ccccc6cc5c3c24)c2ccc3cc4c(cc3c2)oc2ccc3oc5cc6ccccc6cc5c3c24)cc1 ; CC(C)c1ccc(N(c2ccc3cc4c(cc3c2)oc2ccc3oc5cc6ccccc6cc5c3c24)c2ccc3cc4c(cc3c2)oc2ccc3oc5cc6ccccc6cc5c3c24)cc1 ; c1ccc(-c2ccc(N(c3ccc4cc5c(cc4c3)oc3ccc4oc6cc7ccccc7cc6c4c35)c3ccc4cc5c(cc4c3)oc3ccc4oc6cc7ccccc7cc6c4c35)cc2)cc1]

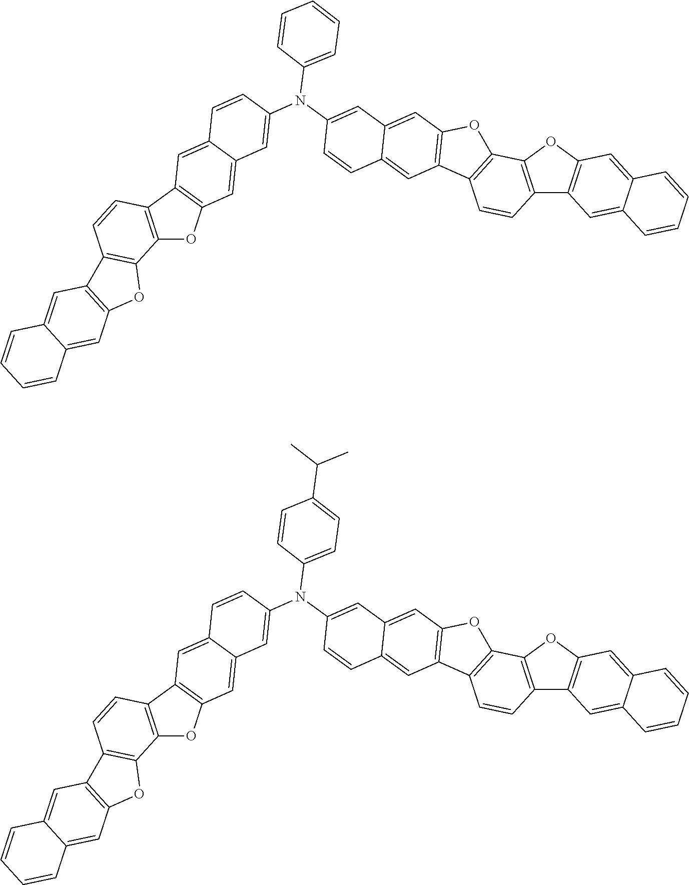 [SMILES: c1ccc(N(c2ccc3cc4c(cc3c2)oc2c4ccc3c4cc5ccccc5cc4oc32)c2ccc3cc4c(cc3c2)oc2c4ccc3c4cc5ccccc5cc4oc32)cc1 ; CC(C)c1ccc(N(c2ccc3cc4c(cc3c2)oc2c4ccc3c4cc5ccccc5cc4oc32)c2ccc3cc4c(cc3c2)oc2c4ccc3c4cc5ccccc5cc4oc32)cc1]

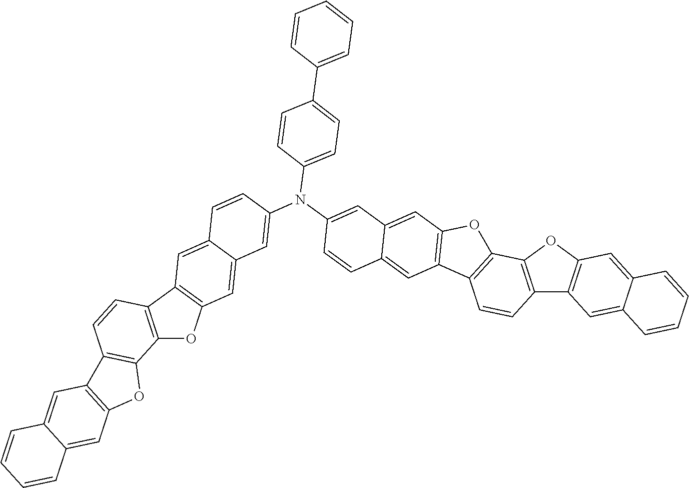 [SMILES: c1ccc(-c2ccc(N(c3ccc4cc5c(cc4c3)oc3c5ccc4c5cc6ccccc6cc5oc43)c3ccc4cc5c(cc4c3)oc3c5ccc4c5cc6ccccc6cc5oc43)cc2)cc1]

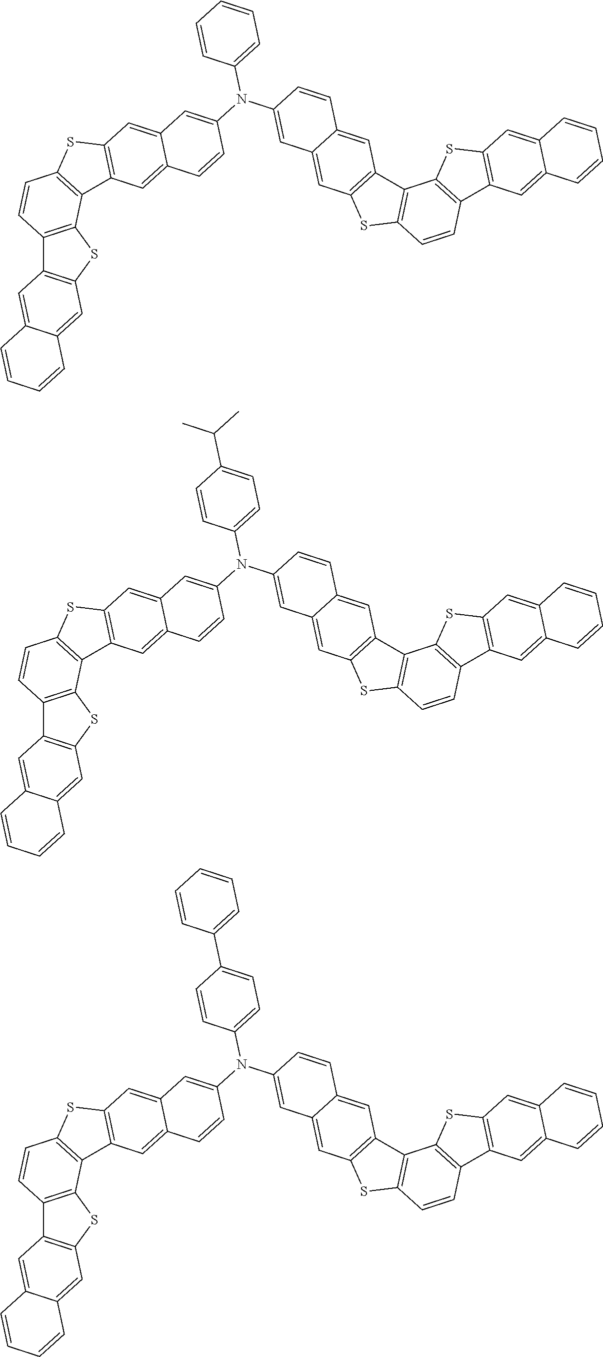 [SMILES: c1ccc(N(c2ccc3cc4c(cc3c2)sc2ccc3c5cc6ccccc6cc5sc3c24)c2ccc3cc4c(cc3c2)sc2ccc3c5cc6ccccc6cc5sc3c24)cc1 ; CC(C)c1ccc(N(c2ccc3cc4c(cc3c2)sc2ccc3c5cc6ccccc6cc5sc3c24)c2ccc3cc4c(cc3c2)sc2ccc3c5cc6ccccc6cc5sc3c24)cc1 ; c1ccc(-c2ccc(N(c3ccc4cc5c(cc4c3)sc3ccc4c6cc7ccccc7cc6sc4c35)c3ccc4cc5c(cc4c3)sc3ccc4c6cc7ccccc7cc6sc4c35)cc2)cc1]

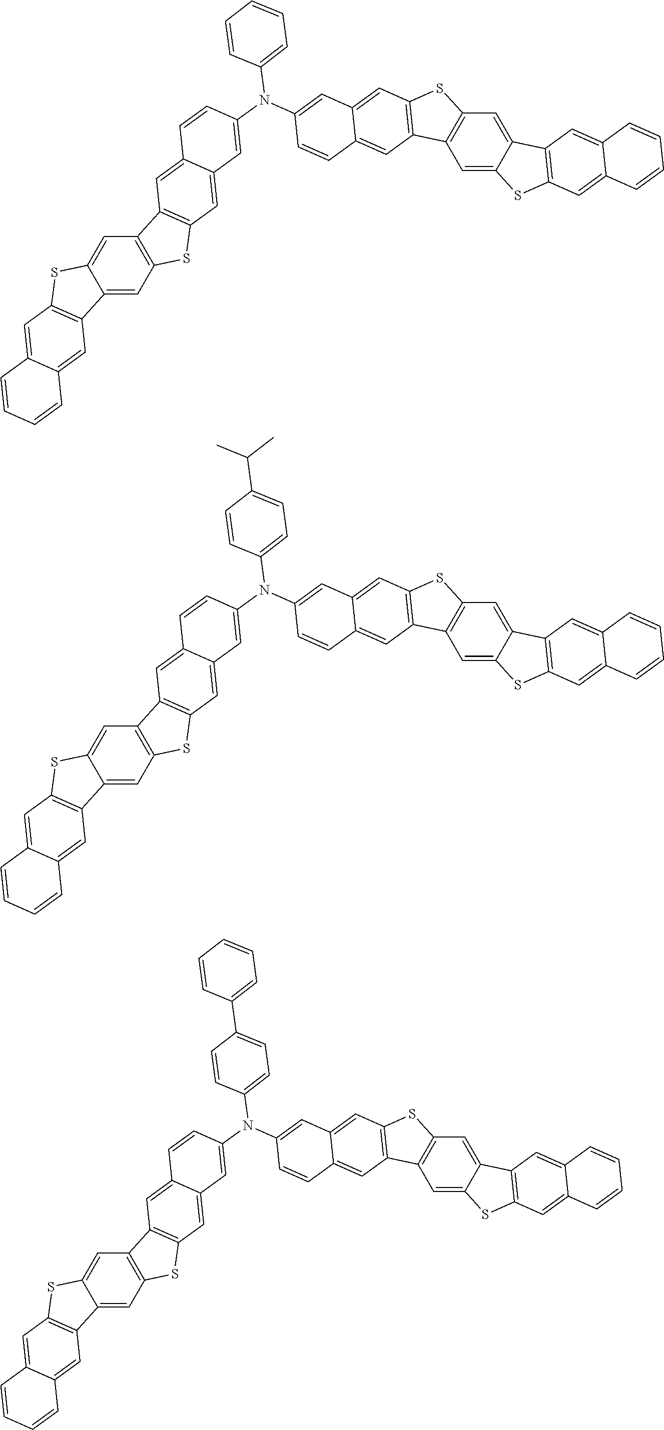 [SMILES: c1ccc(N(c2ccc3cc4c(cc3c2)sc2cc3c(cc24)sc2cc4ccccc4cc23)c2ccc3cc4c(cc3c2)sc2cc3c(cc24)sc2cc4ccccc4cc23)cc1 ; CC(C)c1ccc(N(c2ccc3cc4c(cc3c2)sc2cc3c(cc24)sc2cc4ccccc4cc23)c2ccc3cc4c(cc3c2)sc2cc3c(cc24)sc2cc4ccccc4cc23)cc1 ; c1ccc(-c2ccc(N(c3ccc4cc5c(cc4c3)sc3cc4c(cc35)sc3cc5ccccc5cc34)c3ccc4cc5c(cc4c3)sc3cc4c(cc35)sc3cc5ccccc5cc34)cc2)cc1]

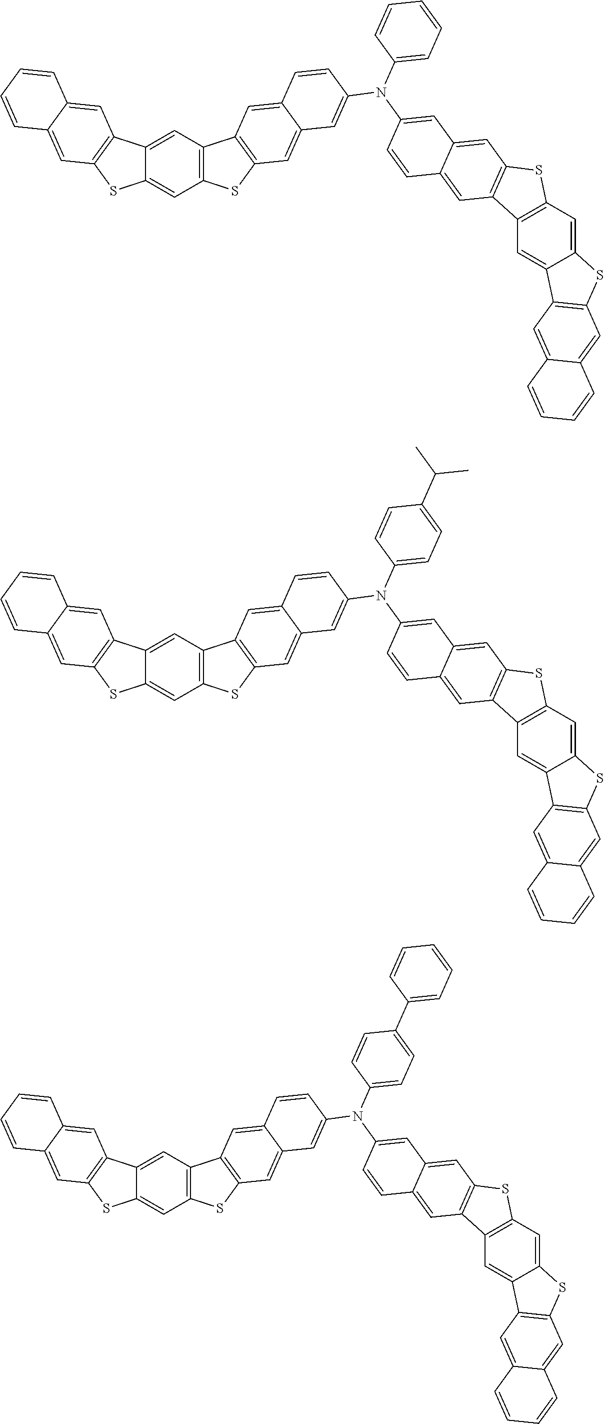 [SMILES: c1ccc(N(c2ccc3cc4c(cc3c2)sc2cc3sc5cc6ccccc6cc5c3cc24)c2ccc3cc4c(cc3c2)sc2cc3sc5cc6ccccc6cc5c3cc24)cc1 ; CC(C)c1ccc(N(c2ccc3cc4c(cc3c2)sc2cc3sc5cc6ccccc6cc5c3cc24)c2ccc3cc4c(cc3c2)sc2cc3sc5cc6ccccc6cc5c3cc24)cc1 ; c1ccc(-c2ccc(N(c3ccc4cc5c(cc4c3)sc3cc4sc6cc7ccccc7cc6c4cc35)c3ccc4cc5c(cc4c3)sc3cc4sc6cc7ccccc7cc6c4cc35)cc2)cc1]

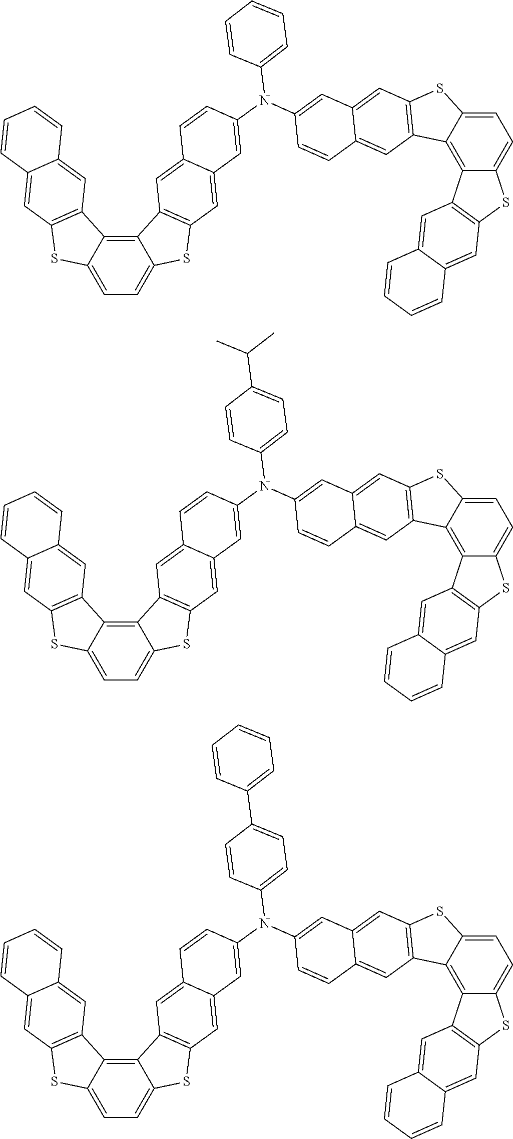 [SMILES: c1ccc(N(c2ccc3cc4c(cc3c2)sc2ccc3sc5cc6ccccc6cc5c3c24)c2ccc3cc4c(cc3c2)sc2ccc3sc5cc6ccccc6cc5c3c24)cc1 ; CC(C)c1ccc(N(c2ccc3cc4c(cc3c2)sc2ccc3sc5cc6ccccc6cc5c3c24)c2ccc3cc4c(cc3c2)sc2ccc3sc5cc6ccccc6cc5c3c24)cc1 ; c1ccc(-c2ccc(N(c3ccc4cc5c(cc4c3)sc3ccc4sc6cc7ccccc7cc6c4c35)c3ccc4cc5c(cc4c3)sc3ccc4sc6cc7ccccc7cc6c4c35)cc2)cc1]

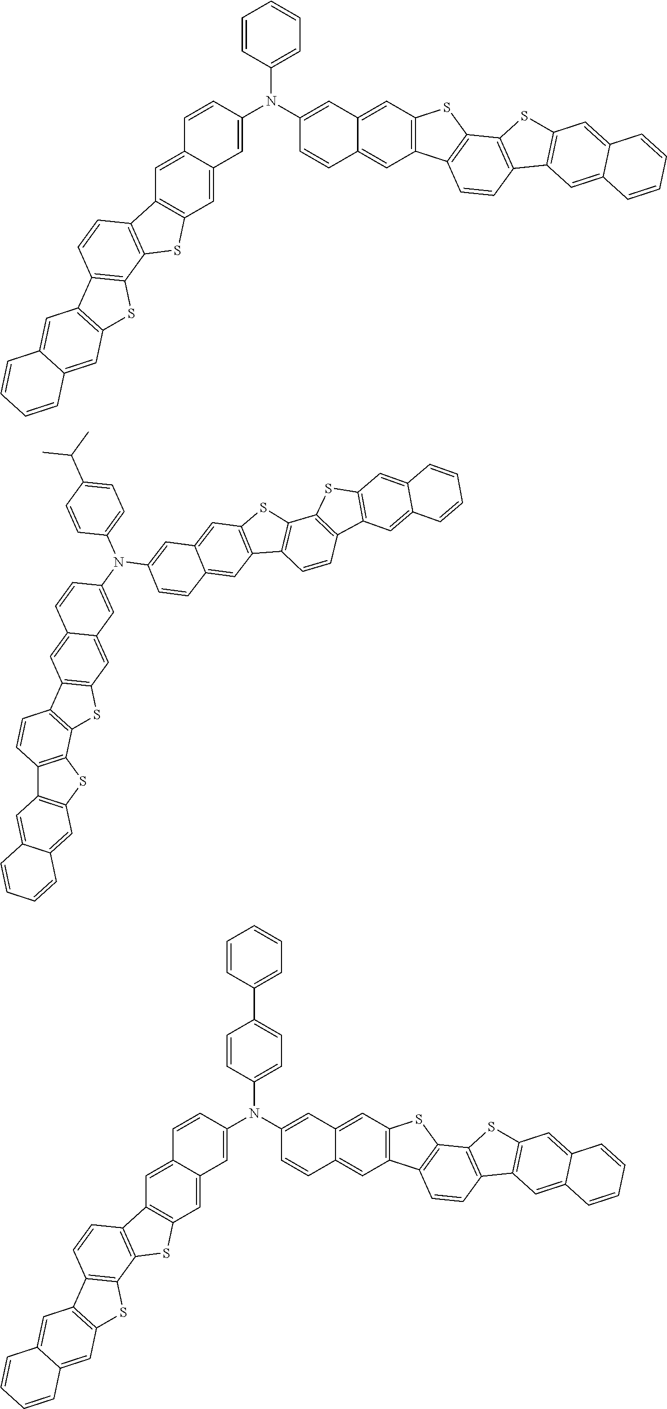

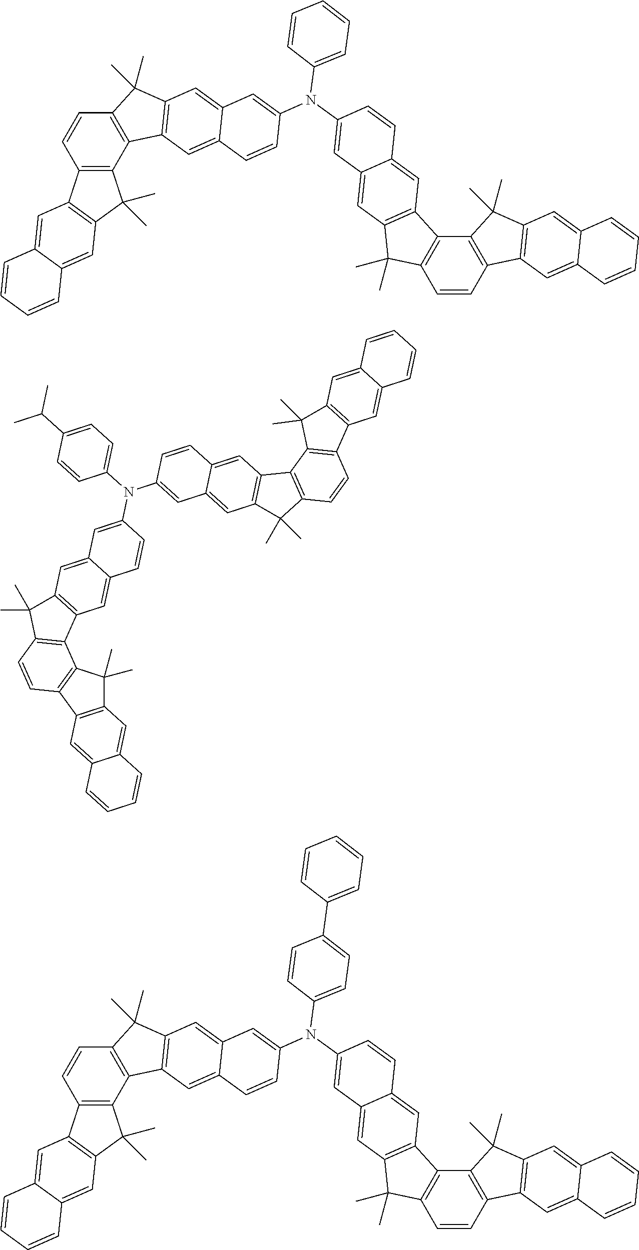

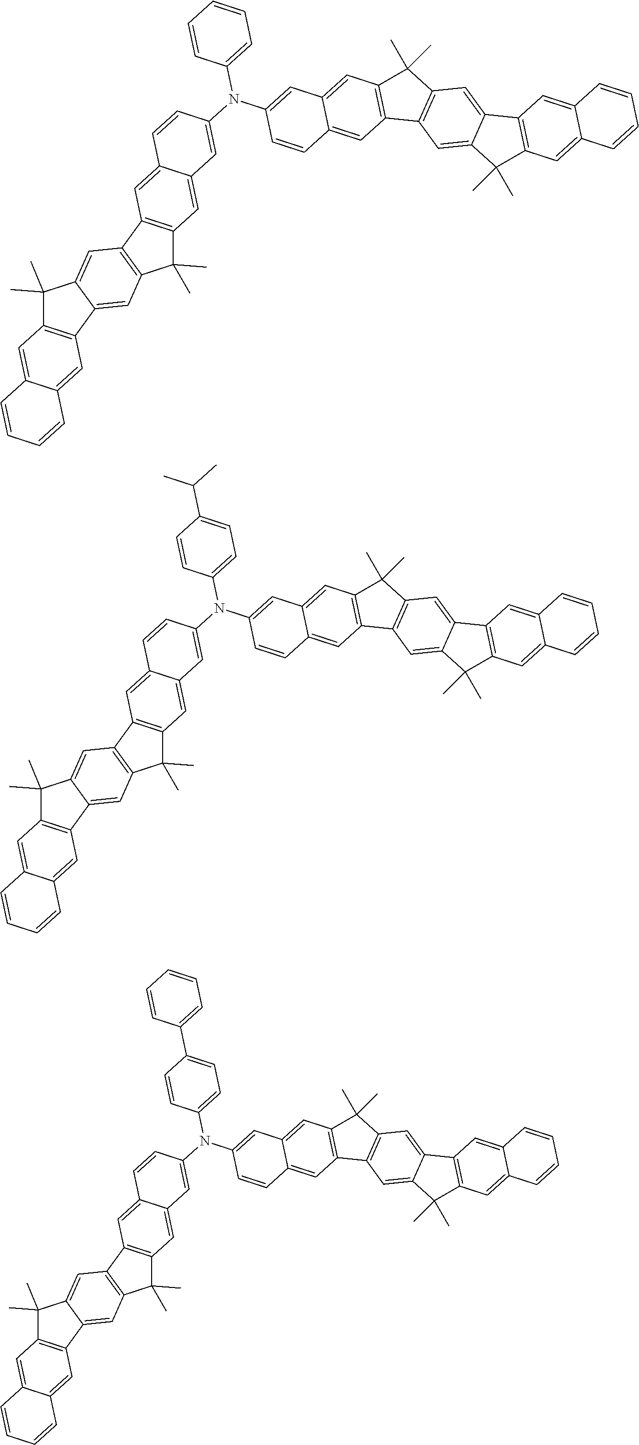 [SMILES: CC1(C)c2cc3c(cc2-c2cc4ccccc4cc21)C(C)(C)c1cc2cc(N(c4ccccc4)c4ccc5cc6c(cc5c4)C(C)(C)c4cc5c(cc4-6)C(C)(C)c4cc6ccccc6cc4-5)ccc2cc1-3 ; CC(C)c1ccc(N(c2ccc3cc4c(cc3c2)C(C)(C)c2cc3c(cc2-4)C(C)(C)c2cc4ccccc4cc2-3)c2ccc3cc4c(cc3c2)C(C)(C)c2cc3c(cc2-4)C(C)(C)c2cc4ccccc4cc2-3)cc1 ; CC1(C)c2cc3c(cc2-c2cc4ccccc4cc21)C(C)(C)c1cc2cc(N(c4ccc(-c5ccccc5)cc4)c4ccc5cc6c(cc5c4)C(C)(C)c4cc5c(cc4-6)C(C)(C)c4cc6ccccc6cc4-5)ccc2cc1-3]

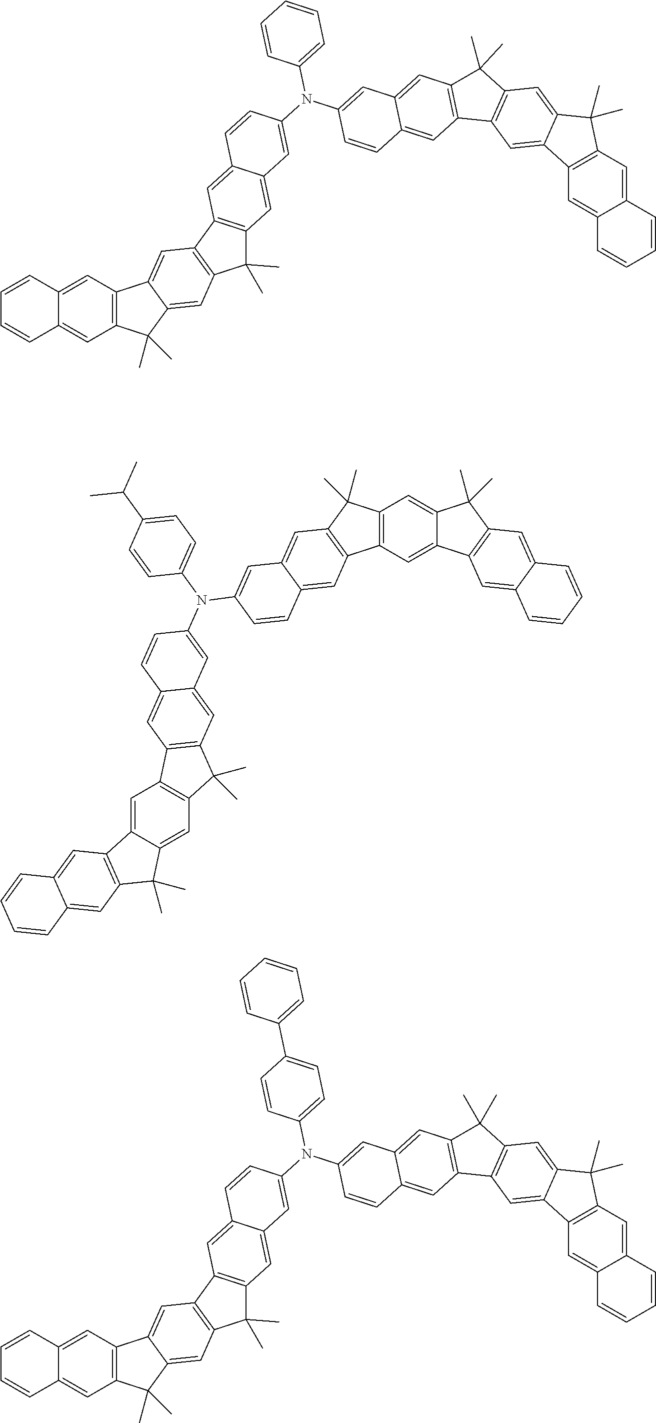 [SMILES: CC1(C)c2cc3c(cc2-c2cc4ccccc4cc21)-c1cc2ccc(N(c4ccccc4)c4ccc5cc6c(cc5c4)C(C)(C)c4cc5c(cc4-6)-c4cc6ccccc6cc4C5(C)C)cc2cc1C3(C)C ; CC(C)c1ccc(N(c2ccc3cc4c(cc3c2)C(C)(C)c2cc3c(cc2-4)-c2cc4ccccc4cc2C3(C)C)c2ccc3cc4c(cc3c2)C(C)(C)c2cc3c(cc2-4)-c2cc4ccccc4cc2C3(C)C)cc1 ; CC1(C)c2cc3c(cc2-c2cc4ccccc4cc21)-c1cc2ccc(N(c4ccc(-c5ccccc5)cc4)c4ccc5cc6c(cc5c4)C(C)(C)c4cc5c(cc4-6)-c4cc6ccccc6cc4C5(C)C)cc2cc1C3(C)C]

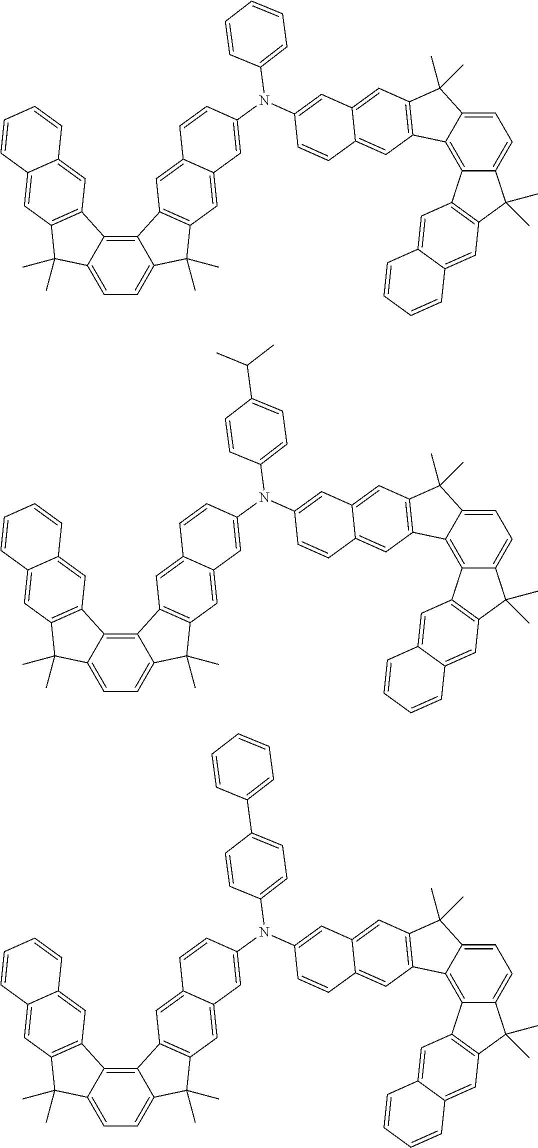 [SMILES: CC1(C)c2cc3ccccc3cc2-c2c1ccc1c2-c2cc3ccc(N(c4ccccc4)c4ccc5cc6c(cc5c4)C(C)(C)c4ccc5c(c4-6)-c4cc6ccccc6cc4C5(C)C)cc3cc2C1(C)C ; CC(C)c1ccc(N(c2ccc3cc4c(cc3c2)C(C)(C)c2ccc3c(c2-4)-c2cc4ccccc4cc2C3(C)C)c2ccc3cc4c(cc3c2)C(C)(C)c2ccc3c(c2-4)-c2cc4ccccc4cc2C3(C)C)cc1 ; CC1(C)c2cc3ccccc3cc2-c2c1ccc1c2-c2cc3ccc(N(c4ccc(-c5ccccc5)cc4)c4ccc5cc6c(cc5c4)C(C)(C)c4ccc5c(c4-6)-c4cc6ccccc6cc4C5(C)C)cc3cc2C1(C)C]

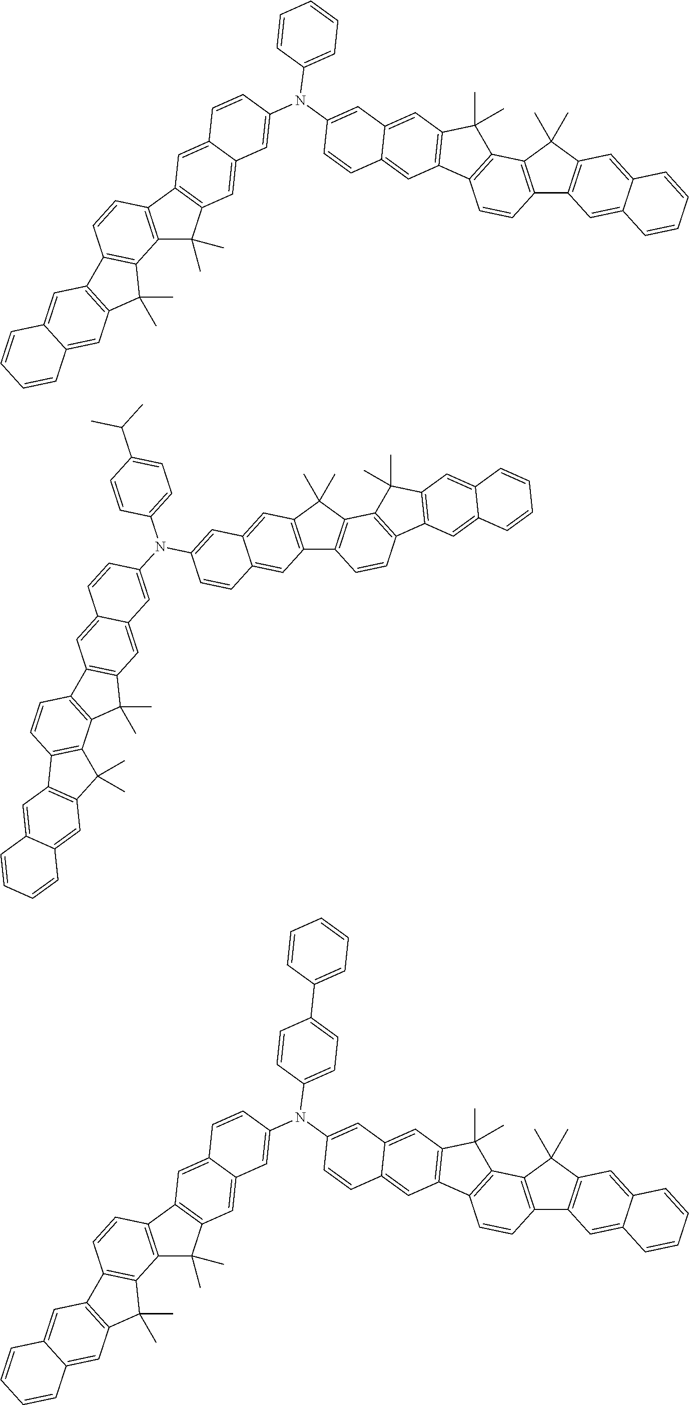 [SMILES: CC1(C)c2cc3ccccc3cc2-c2ccc3c(c21)C(C)(C)c1cc2cc(N(c4ccccc4)c4ccc5cc6c(cc5c4)C(C)(C)c4c-6ccc5c4C(C)(C)c4cc6ccccc6cc4-5)ccc2cc1-3 ; CC(C)c1ccc(N(c2ccc3cc4c(cc3c2)C(C)(C)c2c-4ccc3c2C(C)(C)c2cc4ccccc4cc2-3)c2ccc3cc4c(cc3c2)C(C)(C)c2c-4ccc3c2C(C)(C)c2cc4ccccc4cc2-3)cc1 ; CC1(C)c2cc3ccccc3cc2-c2ccc3c(c21)C(C)(C)c1cc2cc(N(c4ccc(-c5ccccc5)cc4)c4ccc5cc6c(cc5c4)C(C)(C)c4c-6ccc5c4C(C)(C)c4cc6ccccc6cc4-5)ccc2cc1-3]

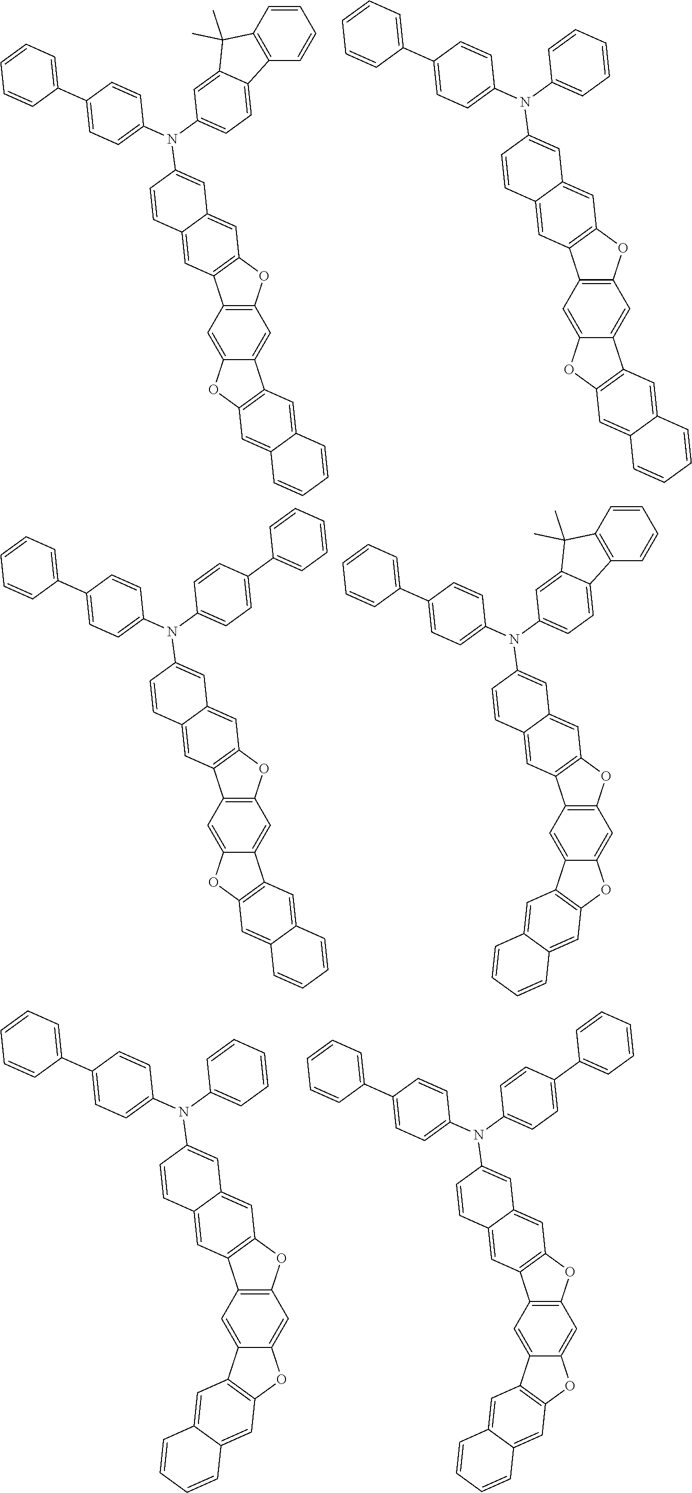

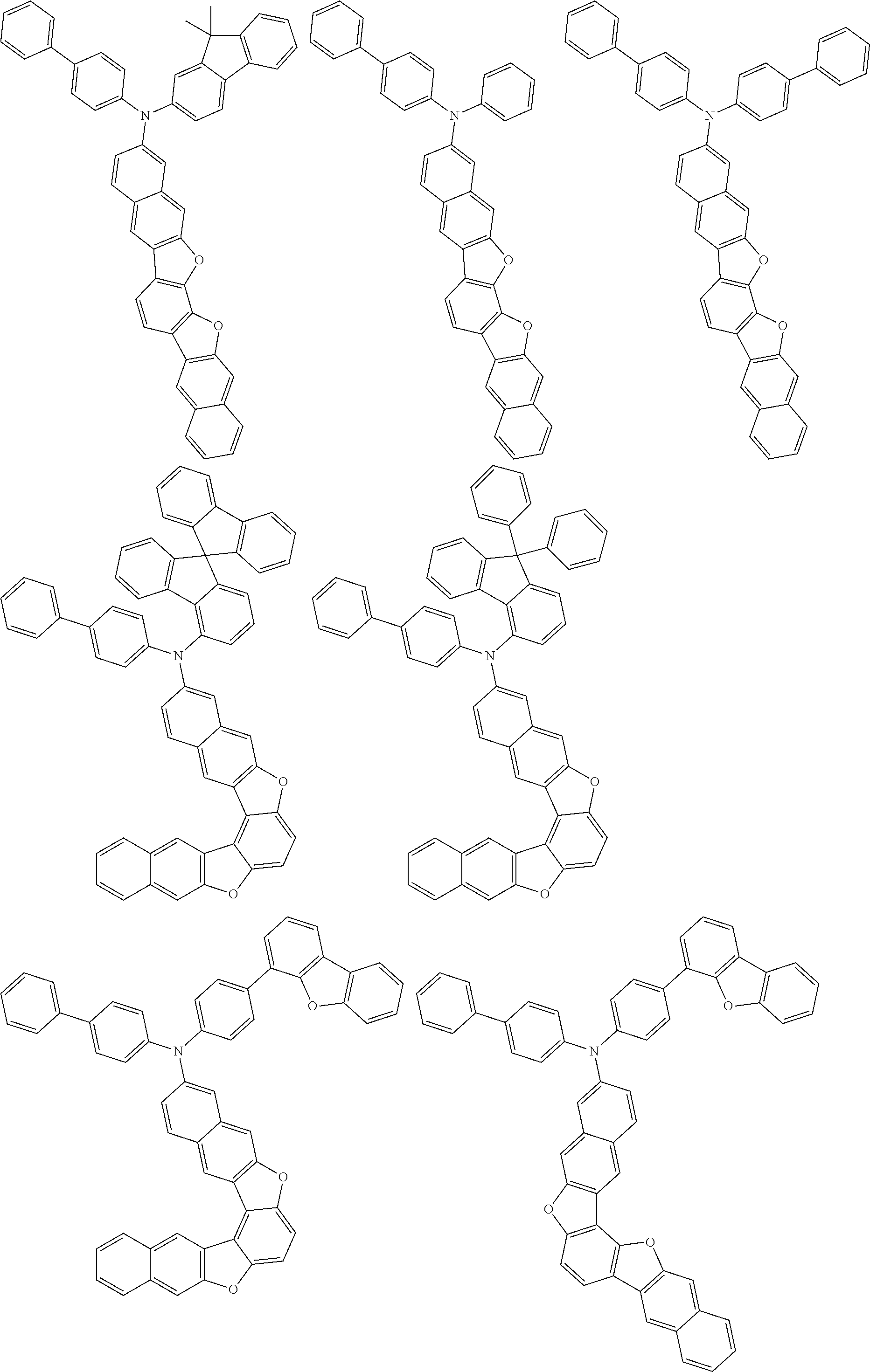

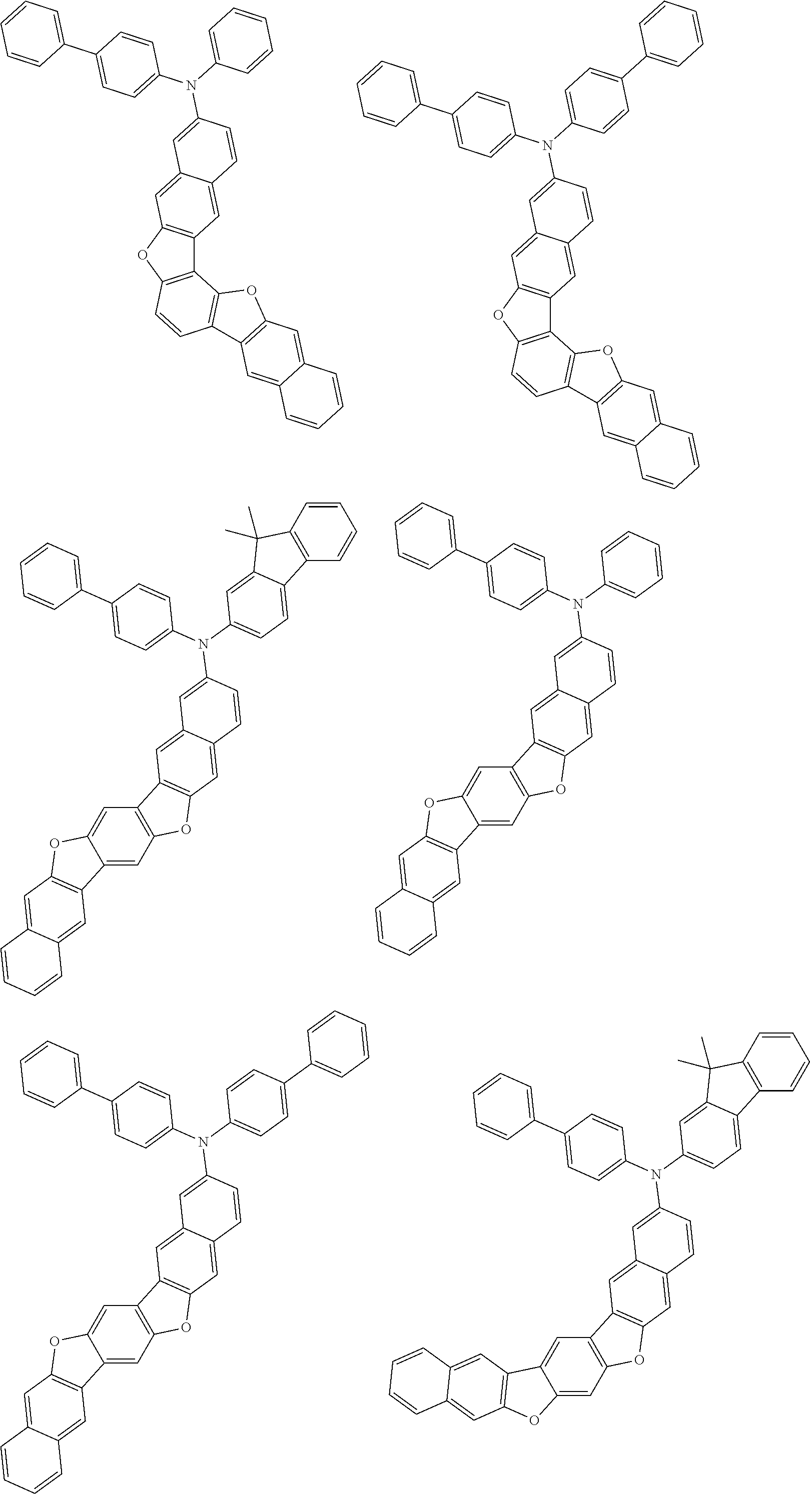

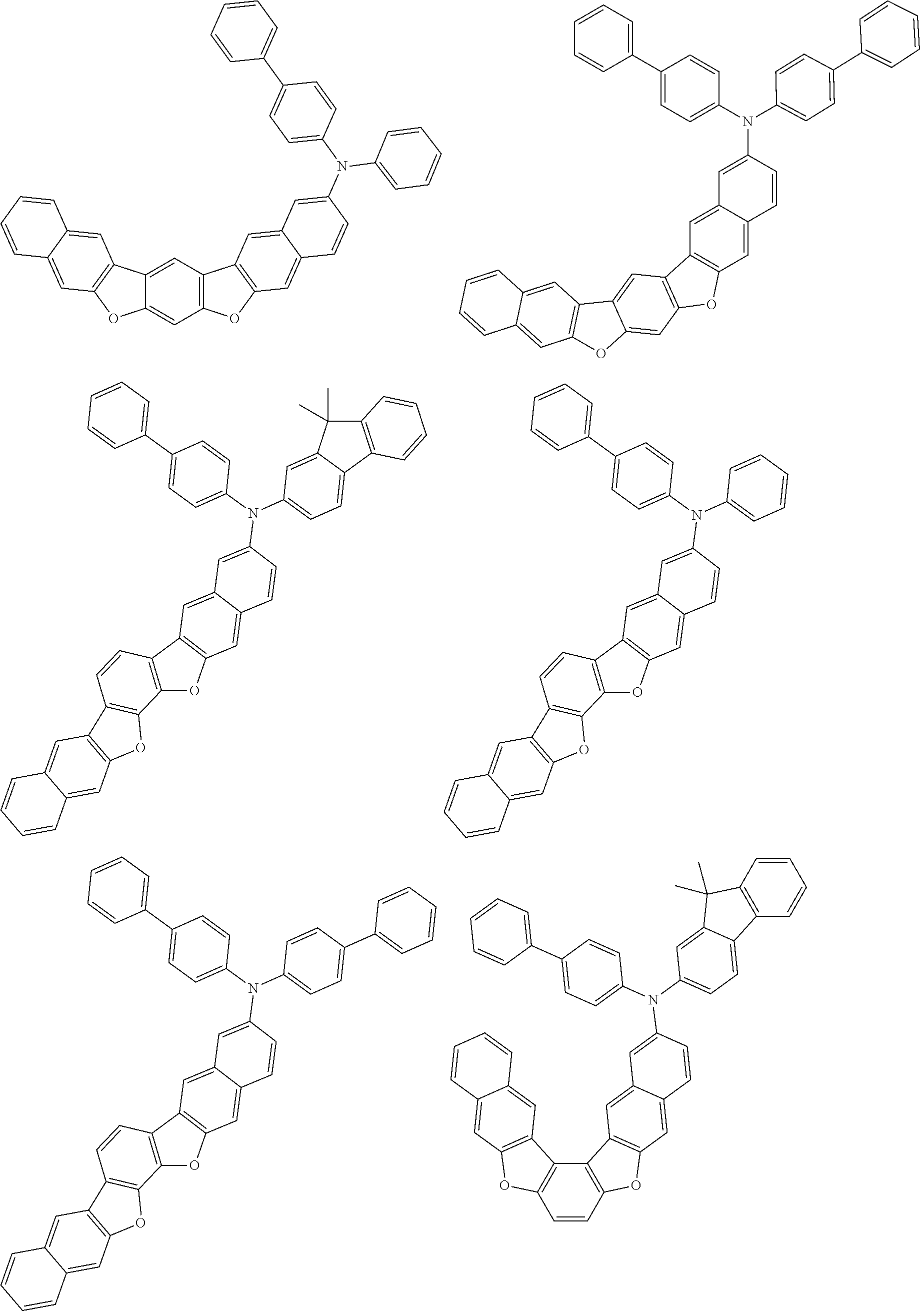

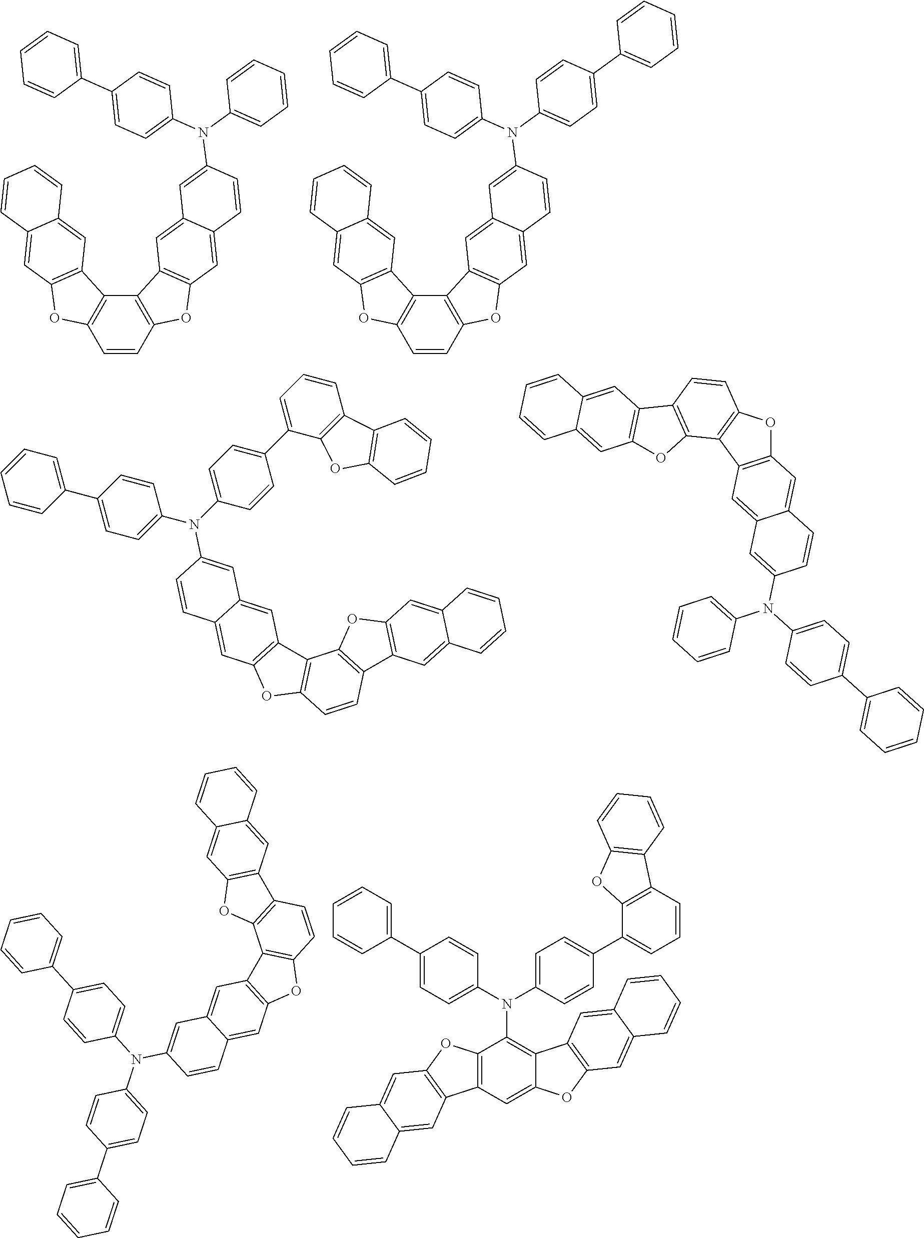

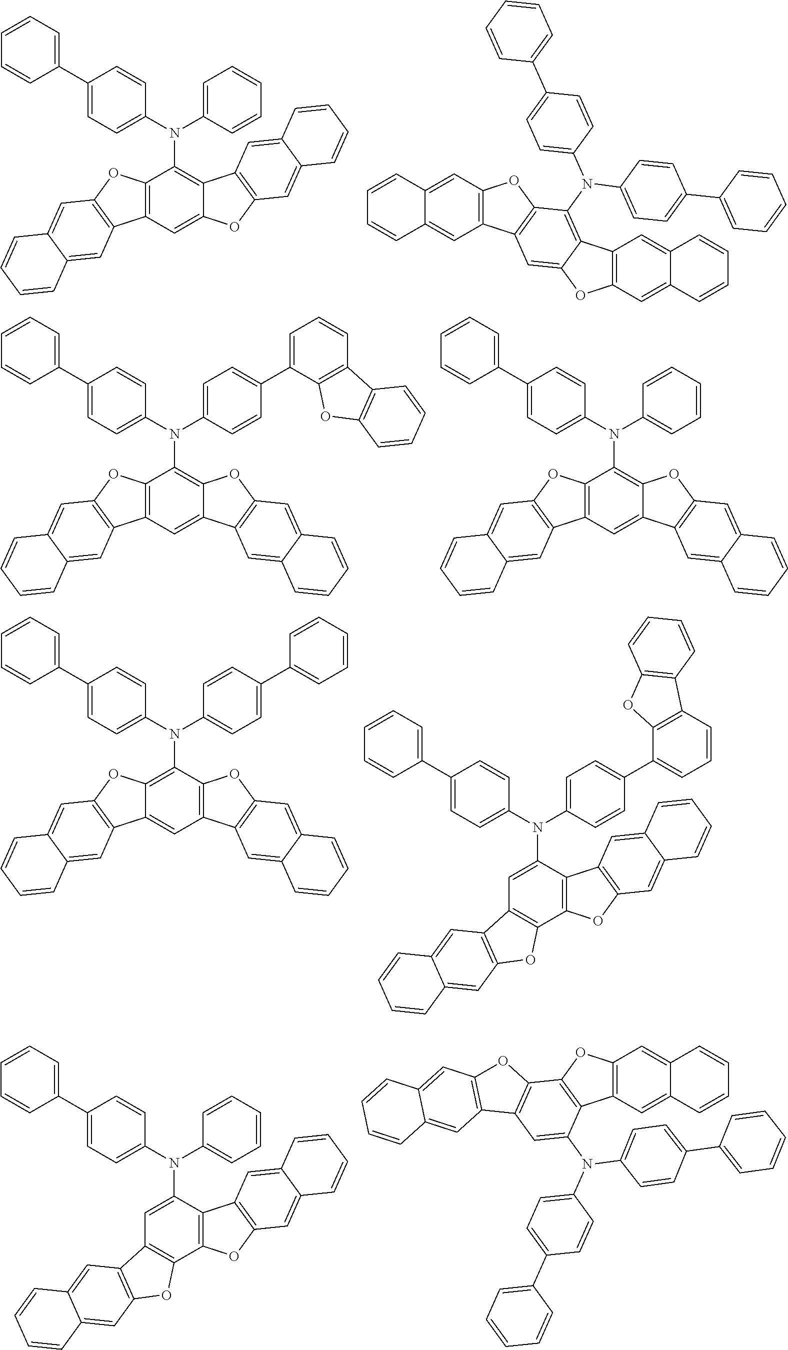

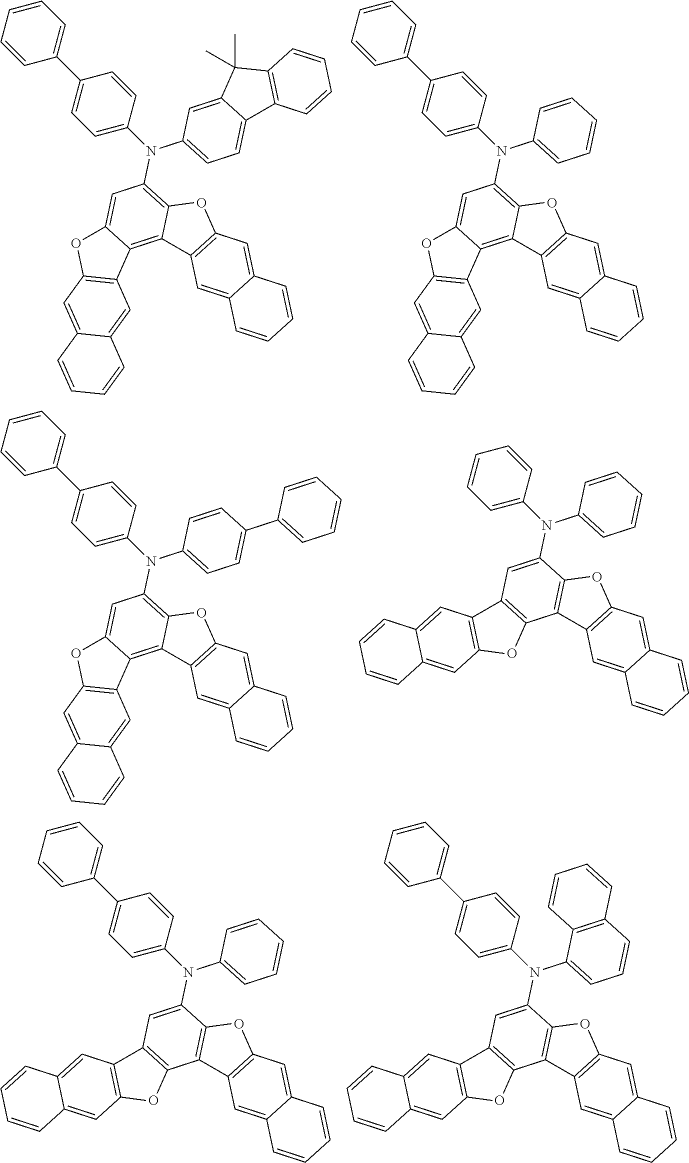

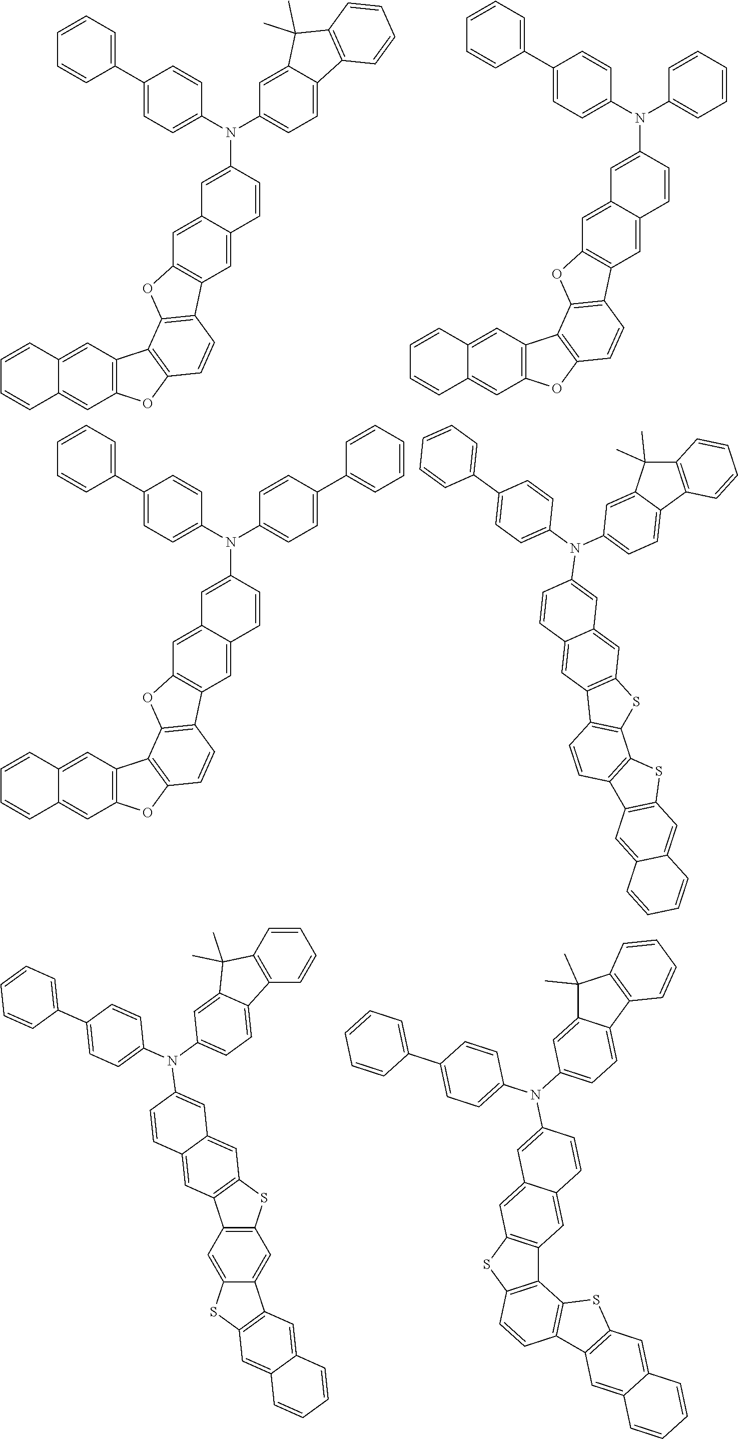

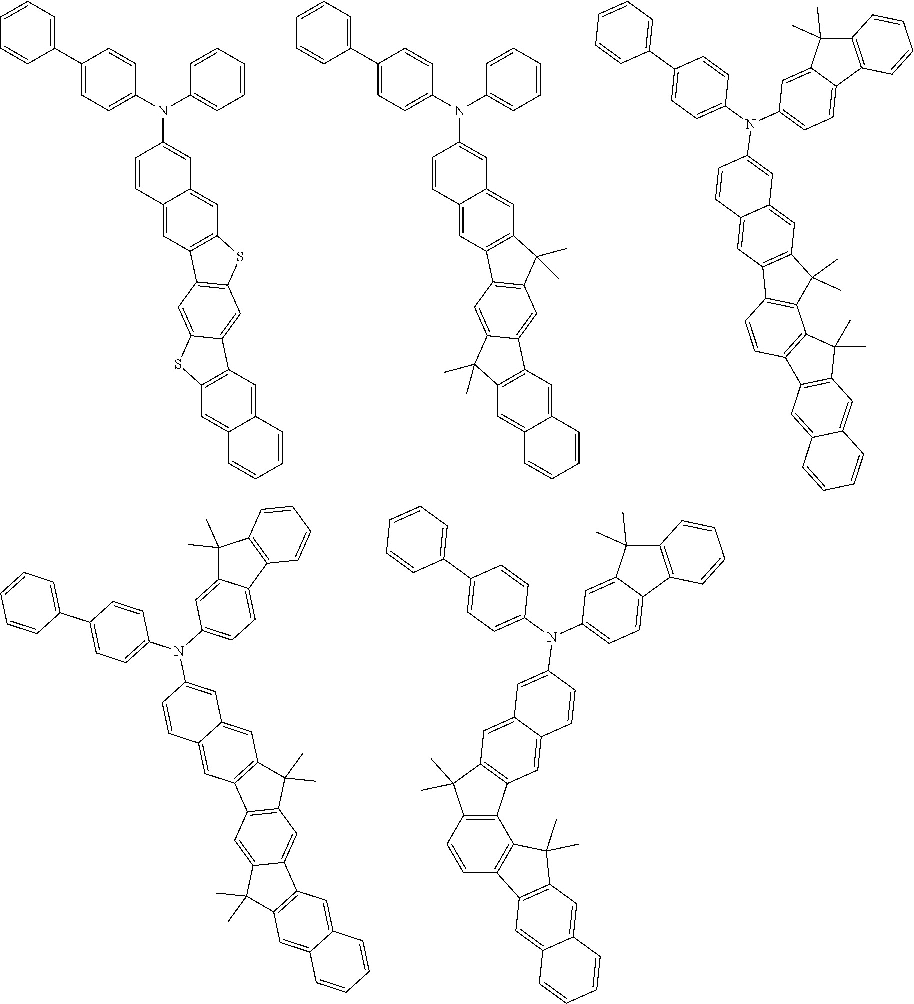

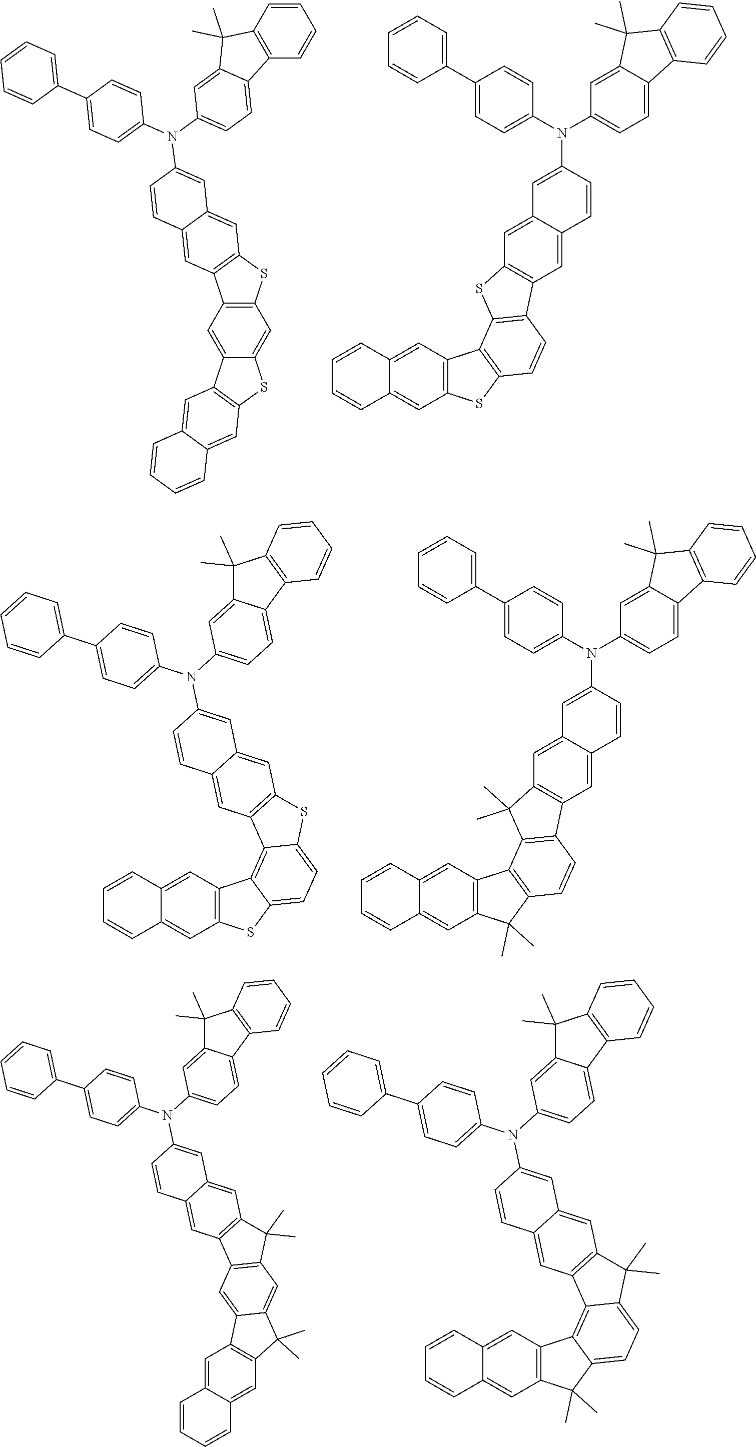

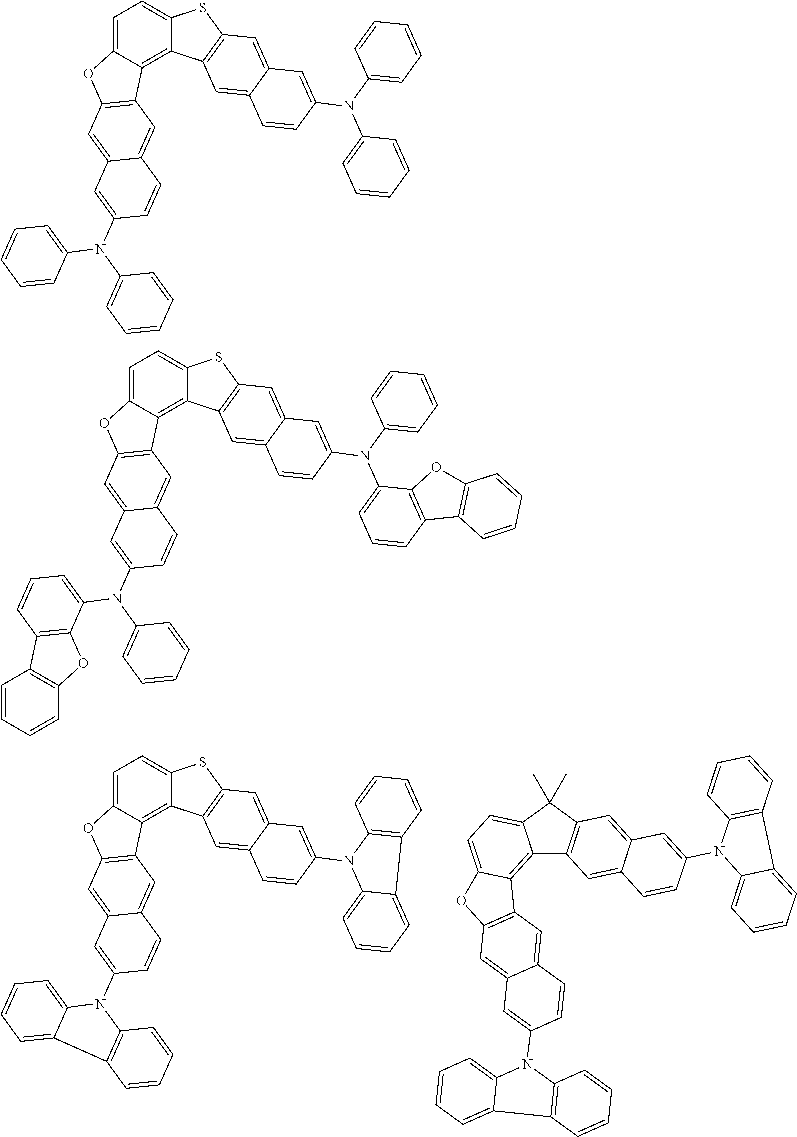

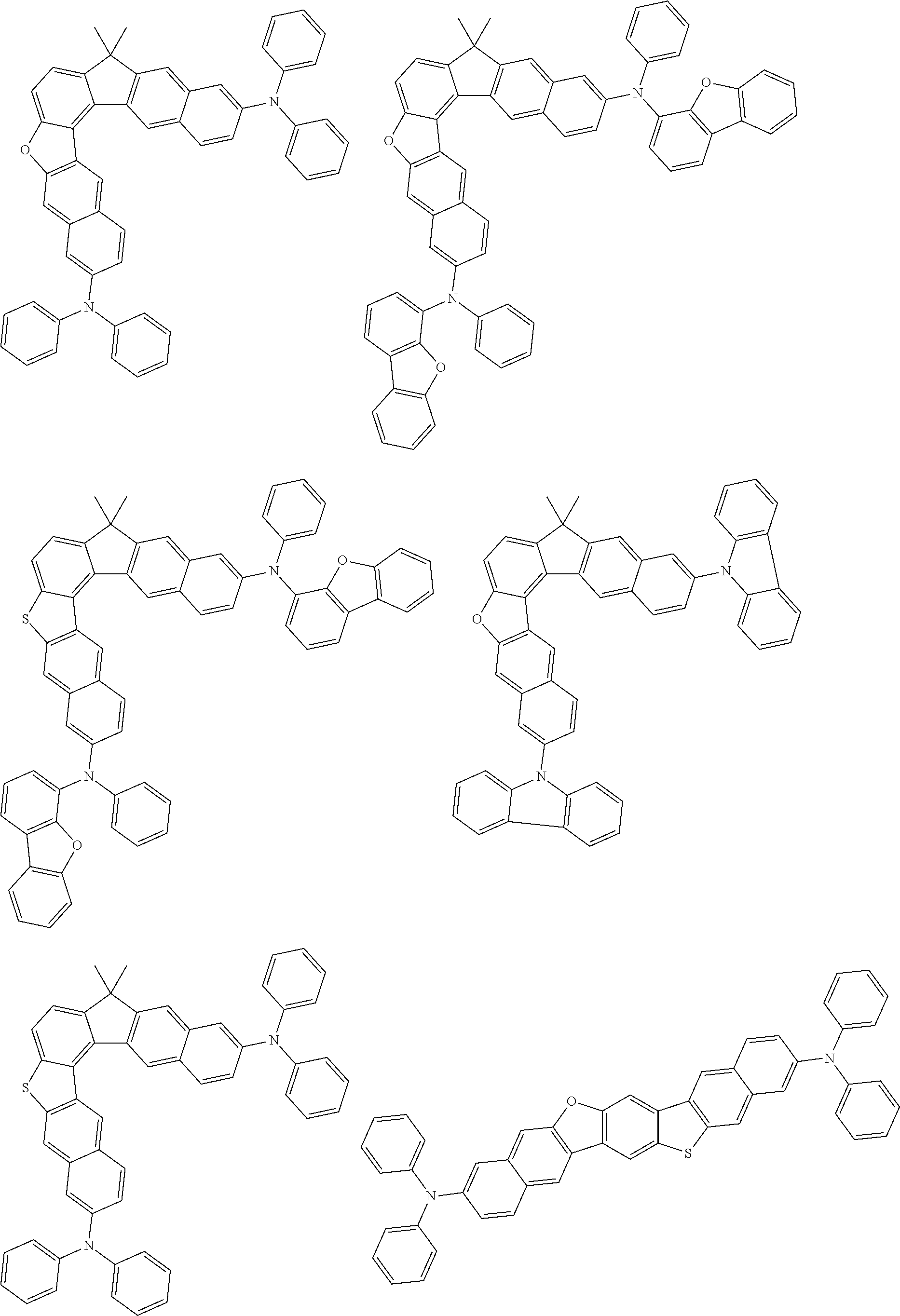

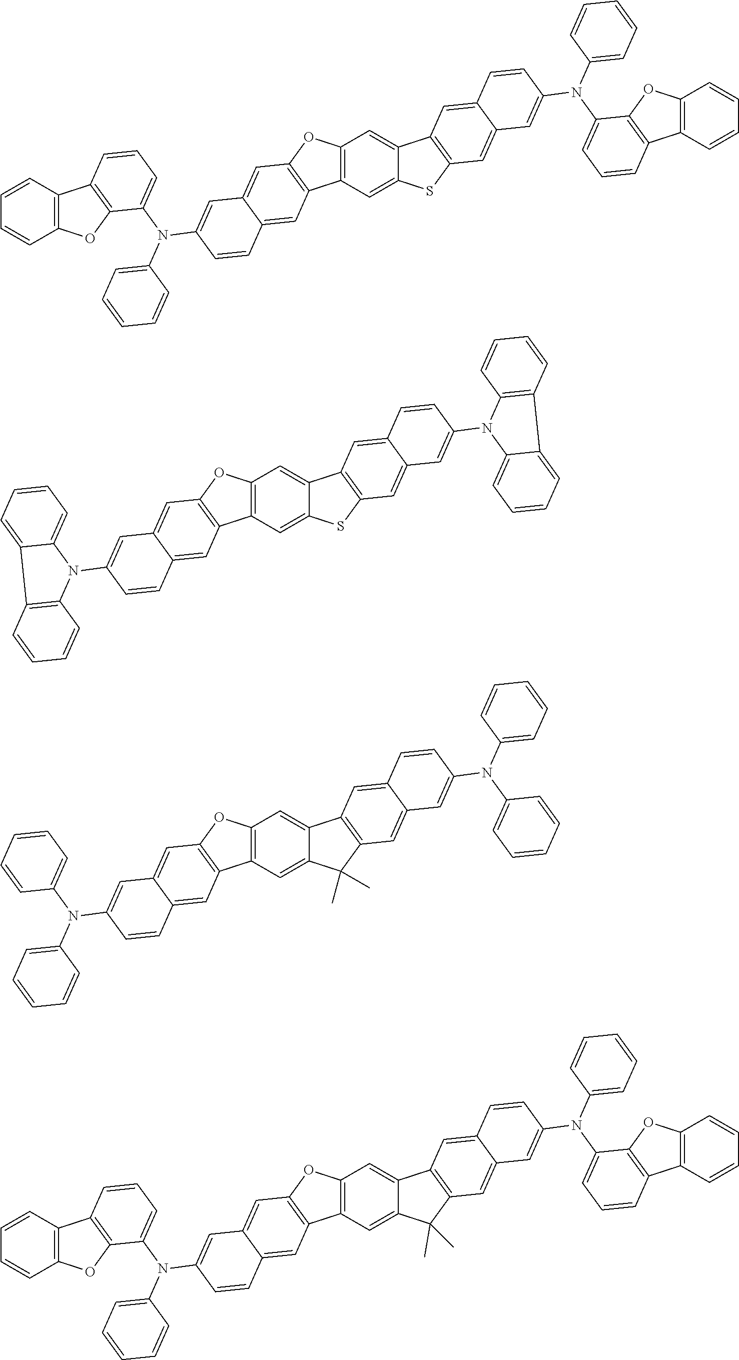 [SMILES: c1ccc(N(c2ccc3cc4c(cc3c2)oc2cc3c(cc24)sc2cc4cc(N(c5ccccc5)c5cccc6c5oc5ccccc56)ccc4cc23)c2cccc3c2oc2ccccc23)cc1 ; c1ccc2c(c1)c1ccccc1n2-c1ccc2cc3c(cc2c1)oc1cc2c(cc13)sc1cc3cc(-n4c5ccccc5c5ccccc54)ccc3cc12 ; CC1(C)c2cc3cc(N(c4ccccc4)c4ccccc4)ccc3cc2-c2cc3oc4cc5cc(N(c6ccccc6)c6ccccc6)ccc5cc4c3cc21 ; CC1(C)c2cc3cc(N(c4ccccc4)c4cccc5c4oc4ccccc45)ccc3cc2-c2cc3oc4cc5cc(N(c6ccccc6)c6cccc7c6oc6ccccc67)ccc5cc4c3cc21]

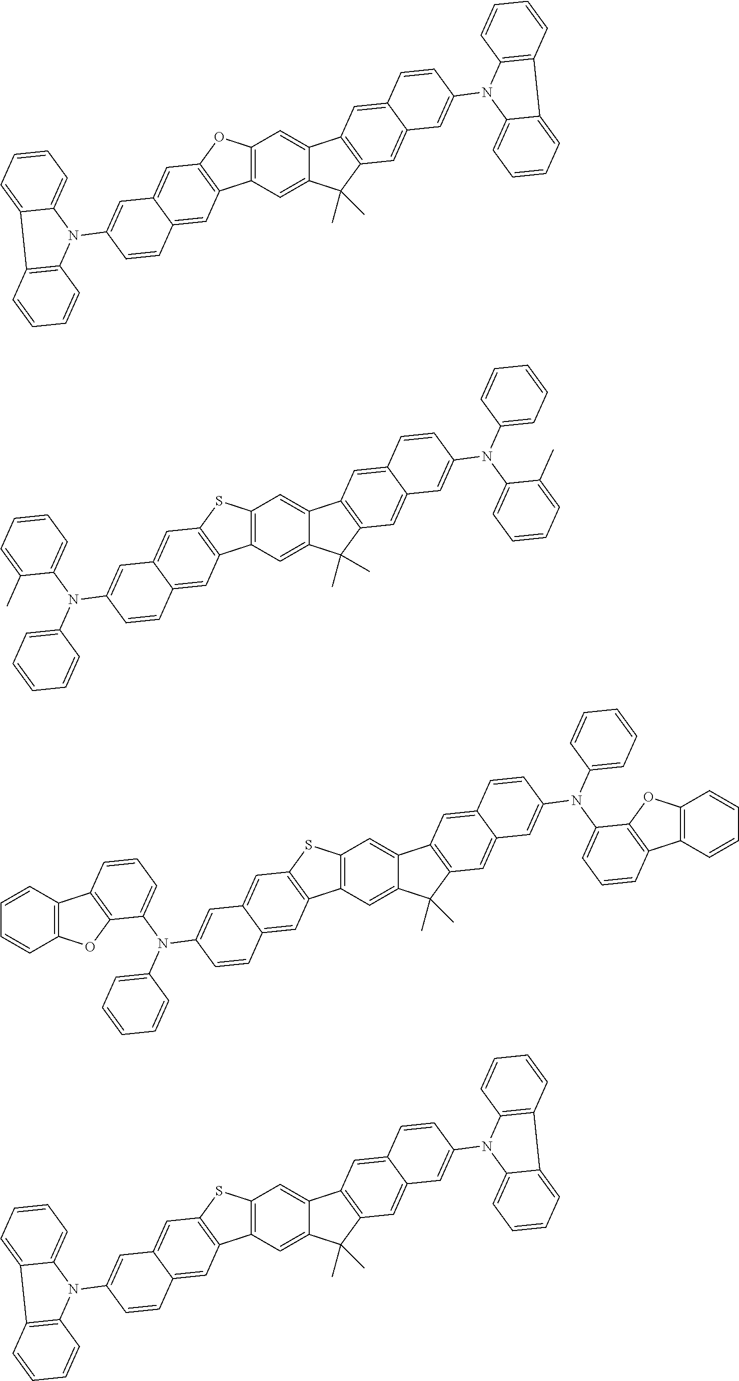 [SMILES: CC1(C)c2cc3cc(-n4c5ccccc5c5ccccc54)ccc3cc2-c2cc3oc4cc5cc(-n6c7ccccc7c7ccccc76)ccc5cc4c3cc21 ; Cc1ccccc1N(c1ccccc1)c1ccc2cc3c(cc2c1)C(C)(C)c1cc2c(cc1-3)sc1cc3cc(N(c4ccccc4)c4ccccc4C)ccc3cc12 ; CC1(C)c2cc3cc(N(c4ccccc4)c4cccc5c4oc4ccccc45)ccc3cc2-c2cc3sc4cc5cc(N(c6ccccc6)c6cccc7c6oc6ccccc67)ccc5cc4c3cc21 ; CC1(C)c2cc3cc(-n4c5ccccc5c5ccccc54)ccc3cc2-c2cc3sc4cc5cc(-n6c7ccccc7c7ccccc76)ccc5cc4c3cc21]

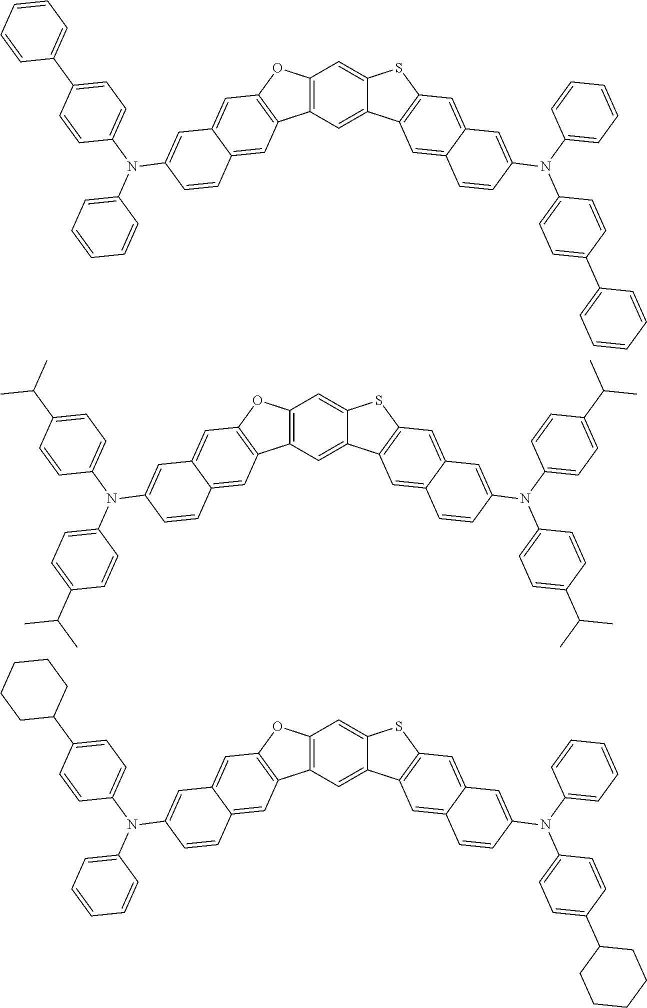

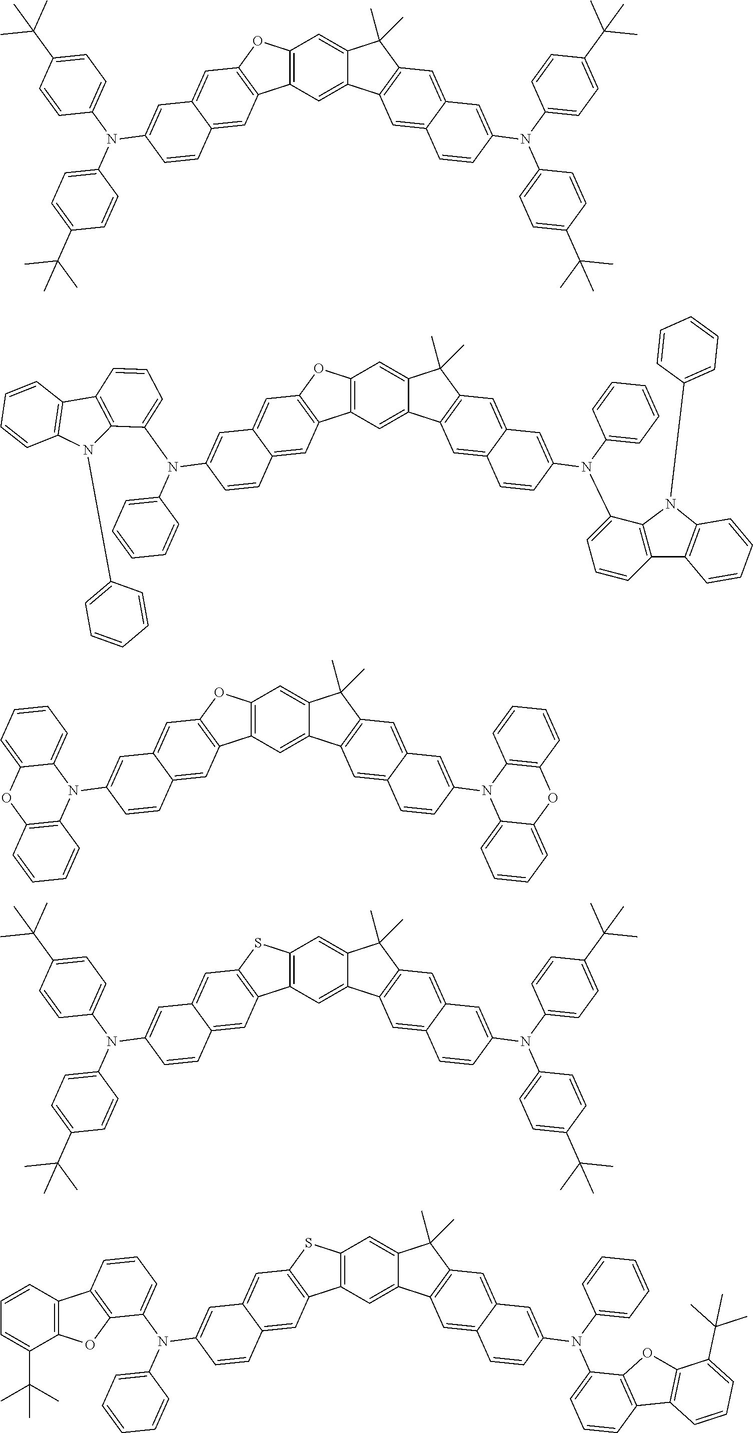

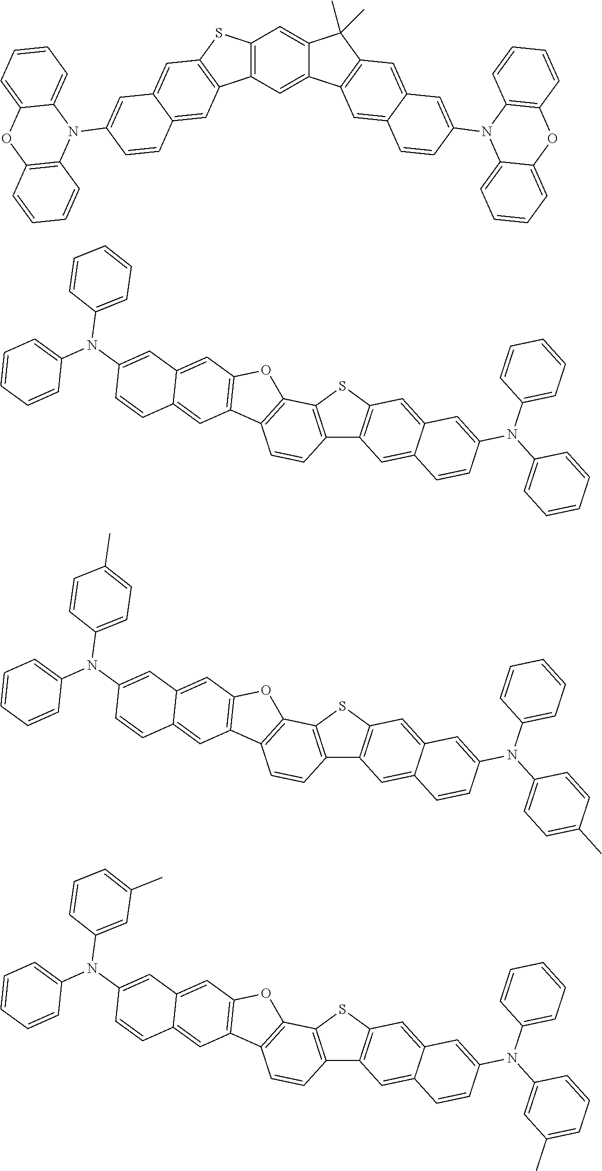

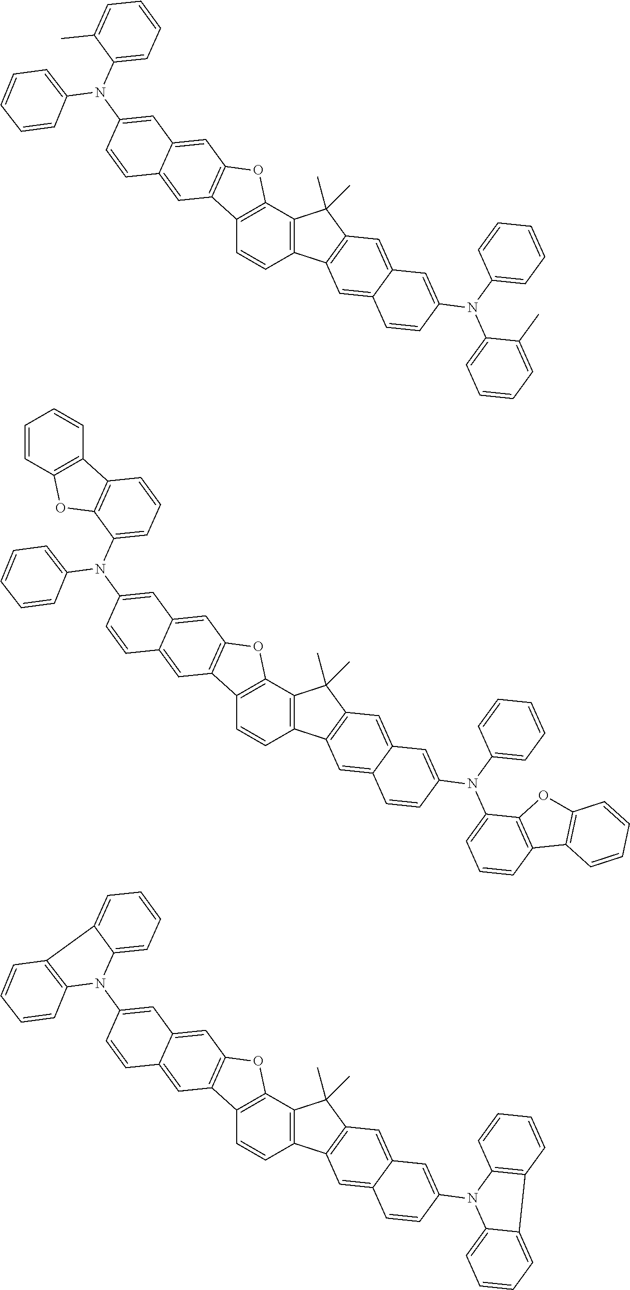 [SMILES: Cc1ccccc1N(c1ccccc1)c1ccc2cc3c(cc2c1)C(C)(C)c1c-3ccc2c1oc1cc3cc(N(c4ccccc4)c4ccccc4C)ccc3cc12 ; CC1(C)c2cc3cc(N(c4ccccc4)c4cccc5c4oc4ccccc45)ccc3cc2-c2ccc3c(oc4cc5cc(N(c6ccccc6)c6cccc7c6oc6ccccc67)ccc5cc43)c21 ; CC1(C)c2cc3cc(-n4c5ccccc5c5ccccc54)ccc3cc2-c2ccc3c(oc4cc5cc(-n6c7ccccc7c7ccccc76)ccc5cc43)c21]

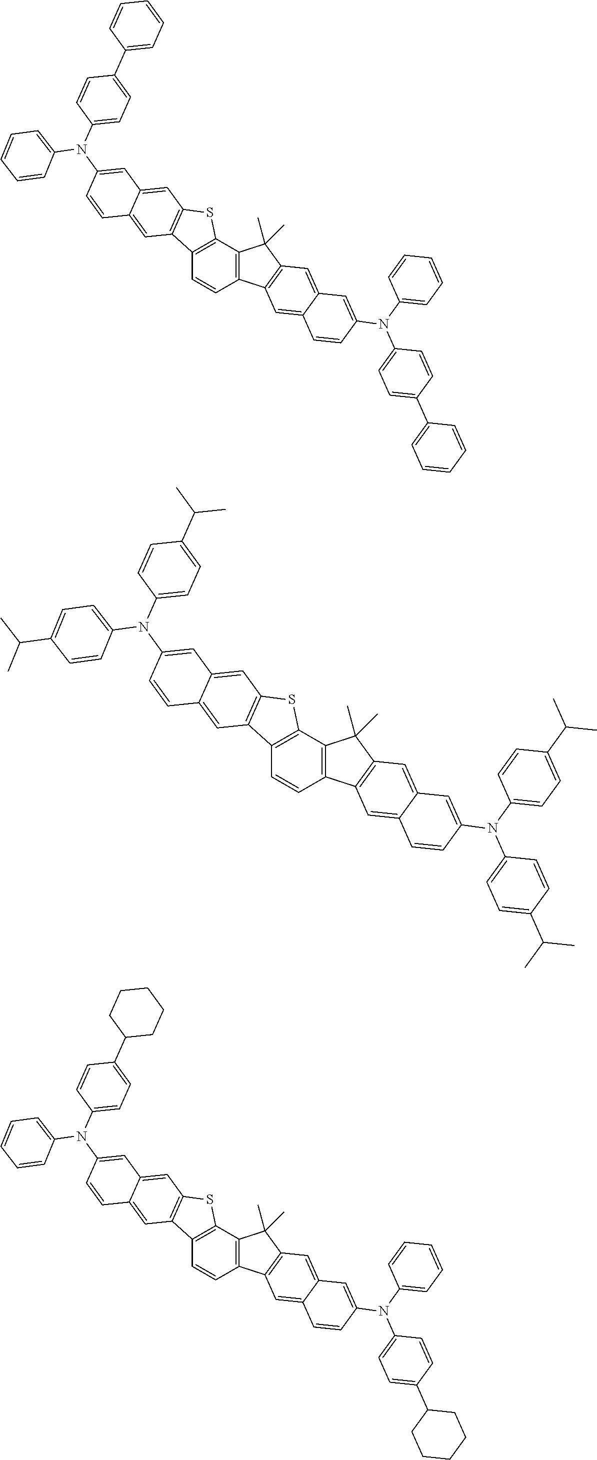 [SMILES: CC1(C)c2cc3cc(N(c4ccccc4)c4ccc(-c5ccccc5)cc4)ccc3cc2-c2ccc3c(sc4cc5cc(N(c6ccccc6)c6ccc(-c7ccccc7)cc6)ccc5cc43)c21 ; CC(C)c1ccc(N(c2ccc(C(C)C)cc2)c2ccc3cc4c(cc3c2)C(C)(C)c2c-4ccc3c2sc2cc4cc(N(c5ccc(C(C)C)cc5)c5ccc(C(C)C)cc5)ccc4cc23)cc1 ; CC1(C)c2cc3cc(N(c4ccccc4)c4ccc(C5CCCCC5)cc4)ccc3cc2-c2ccc3c(sc4cc5cc(N(c6ccccc6)c6ccc(C7CCCCC7)cc6)ccc5cc43)c21]

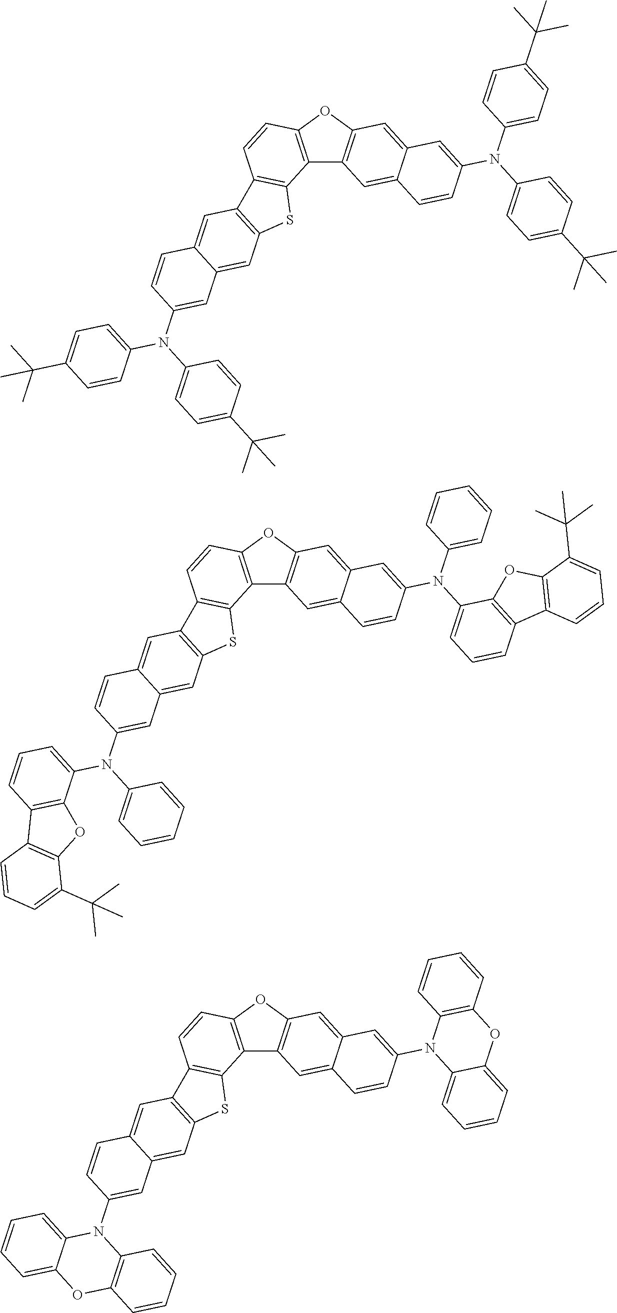 [SMILES: CC(C)(C)c1ccc(N(c2ccc(C(C)(C)C)cc2)c2ccc3cc4c(cc3c2)oc2ccc3c5cc6ccc(N(c7ccc(C(C)(C)C)cc7)c7ccc(C(C)(C)C)cc7)cc6cc5sc3c24)cc1 ; CC(C)(C)c1cccc2c1oc1c(N(c3ccccc3)c3ccc4cc5c(cc4c3)oc3ccc4c6cc7ccc(N(c8ccccc8)c8cccc9c8oc8c(C(C)(C)C)cccc89)cc7cc6sc4c35)cccc12 ; c1ccc2c(c1)Oc1ccccc1N2c1ccc2cc3c(cc2c1)oc1ccc2c4cc5ccc(N6c7ccccc7Oc7ccccc76)cc5cc4sc2c13]

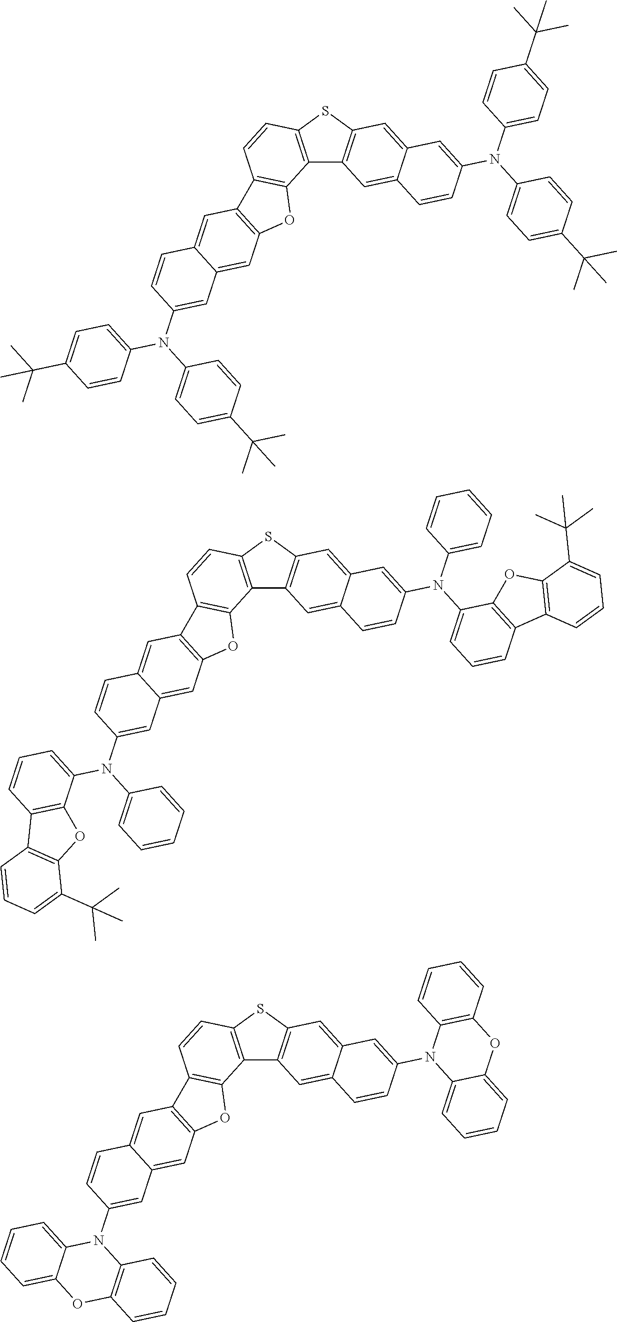 [SMILES: CC(C)(C)c1ccc(N(c2ccc(C(C)(C)C)cc2)c2ccc3cc4c(cc3c2)oc2c4ccc3sc4cc5cc(N(c6ccc(C(C)(C)C)cc6)c6ccc(C(C)(C)C)cc6)ccc5cc4c32)cc1 ; CC(C)(C)c1cccc2c1oc1c(N(c3ccccc3)c3ccc4cc5c(cc4c3)oc3c5ccc4sc5cc6cc(N(c7ccccc7)c7cccc8c7oc7c(C(C)(C)C)cccc78)ccc6cc5c43)cccc12 ; c1ccc2c(c1)Oc1ccccc1N2c1ccc2cc3c(cc2c1)oc1c3ccc2sc3cc4cc(N5c6ccccc6Oc6ccccc65)ccc4cc3c21]

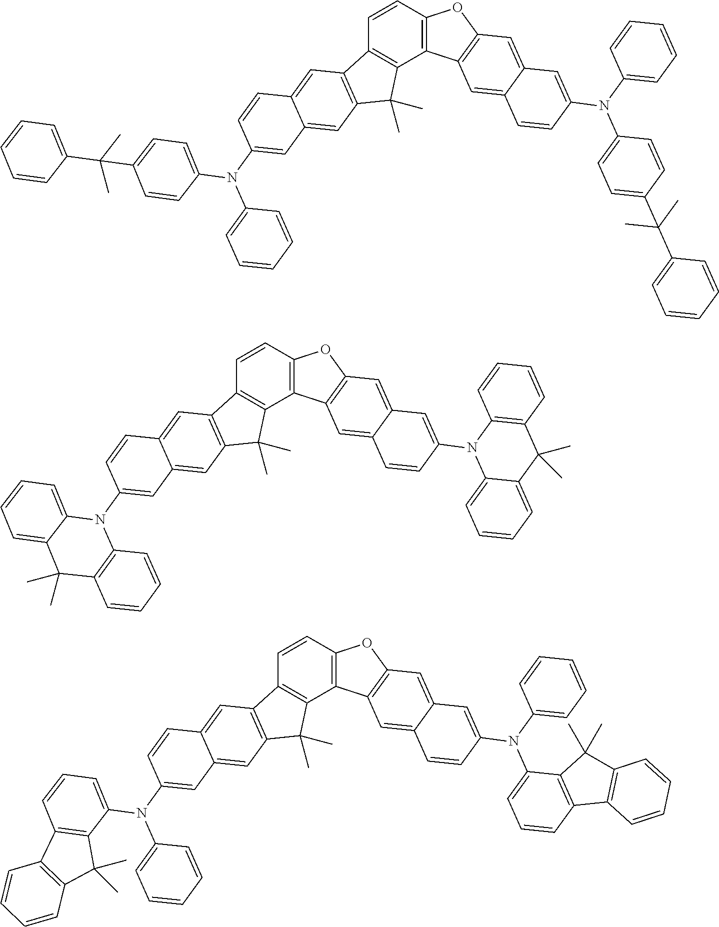 [SMILES: CC(C)(c1ccccc1)c1ccc(N(c2ccccc2)c2ccc3cc4c(cc3c2)C(C)(C)c2c-4ccc3oc4cc5cc(N(c6ccccc6)c6ccc(C(C)(C)c7ccccc7)cc6)ccc5cc4c23)cc1 ; CC1(C)c2ccccc2N(c2ccc3cc4c(cc3c2)C(C)(C)c2c-4ccc3oc4cc5cc(N6c7ccccc7C(C)(C)c7ccccc76)ccc5cc4c23)c2ccccc21 ; CC1(C)c2ccccc2-c2cccc(N(c3ccccc3)c3ccc4cc5c(cc4c3)C(C)(C)c3c-5ccc4oc5cc6cc(N(c7ccccc7)c7cccc8c7C(C)(C)c7ccccc7-8)ccc6cc5c34)c21]

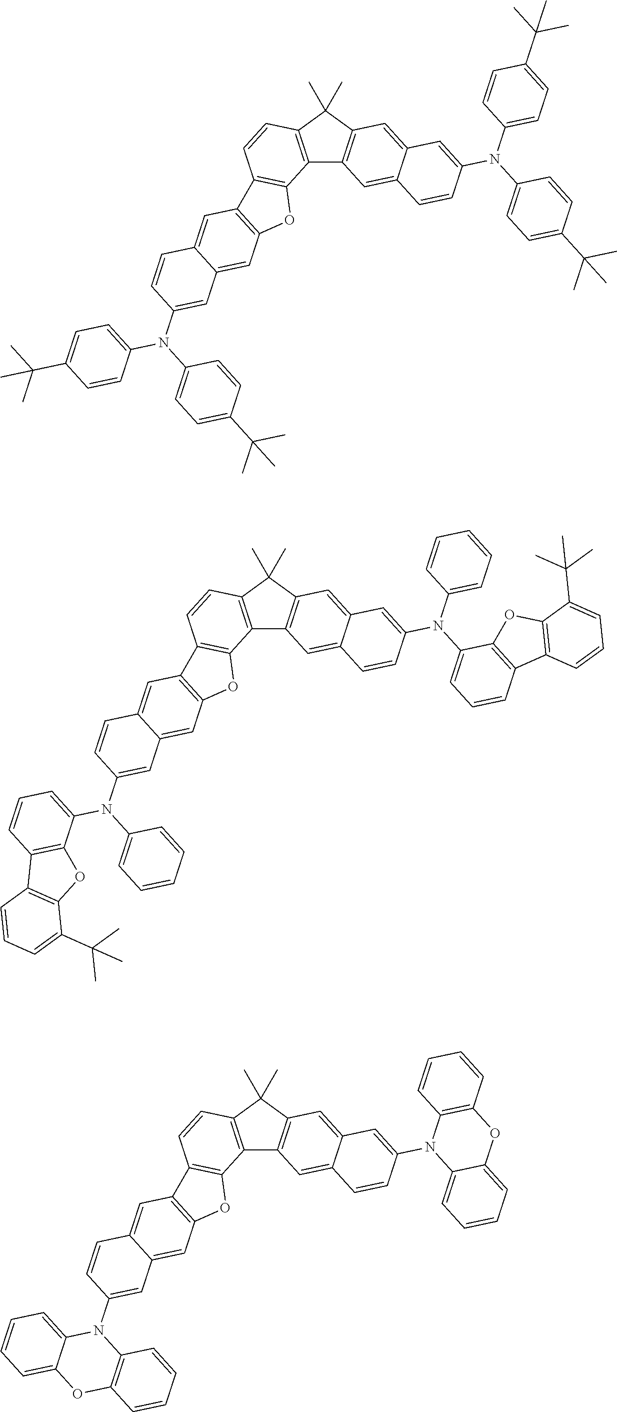 [SMILES: CC(C)(C)c1ccc(N(c2ccc(C(C)(C)C)cc2)c2ccc3cc4c(cc3c2)C(C)(C)c2ccc3c(oc5cc6cc(N(c7ccc(C(C)(C)C)cc7)c7ccc(C(C)(C)C)cc7)ccc6cc53)c2-4)cc1 ; CC(C)(C)c1cccc2c1oc1c(N(c3ccccc3)c3ccc4cc5c(cc4c3)C(C)(C)c3ccc4c(oc6cc7cc(N(c8ccccc8)c8cccc9c8oc8c(C(C)(C)C)cccc89)ccc7cc64)c3-5)cccc12 ; CC1(C)c2cc3cc(N4c5ccccc5Oc5ccccc54)ccc3cc2-c2c1ccc1c2oc2cc3cc(N4c5ccccc5Oc5ccccc54)ccc3cc21]

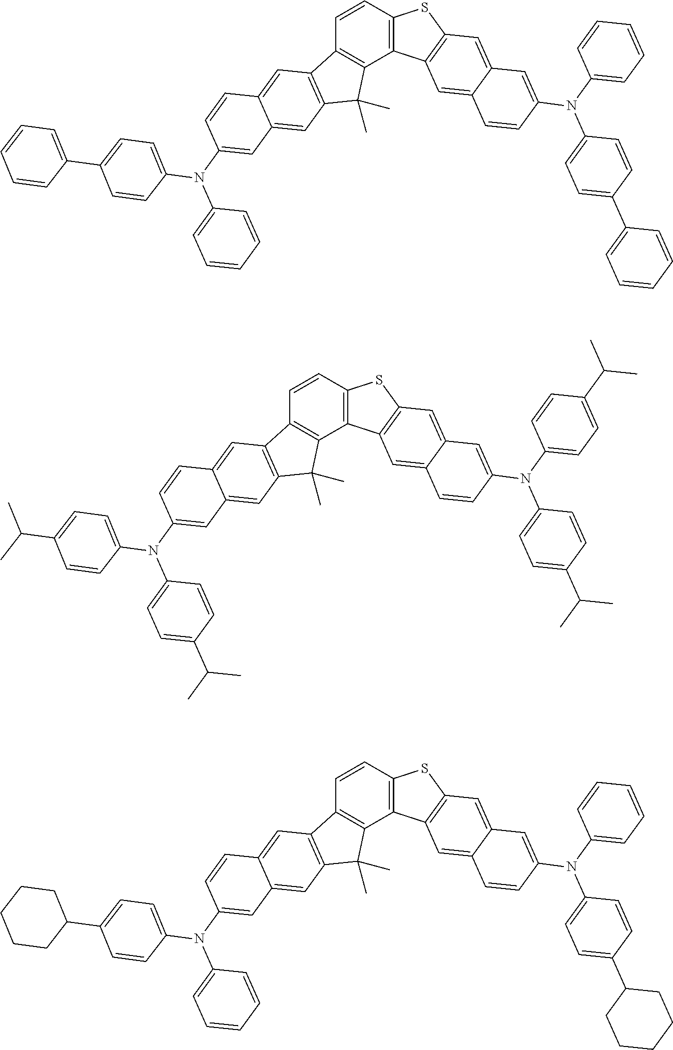 [SMILES: CC1(C)c2cc3cc(N(c4ccccc4)c4ccc(-c5ccccc5)cc4)ccc3cc2-c2ccc3sc4cc5cc(N(c6ccccc6)c6ccc(-c7ccccc7)cc6)ccc5cc4c3c21 ; CC(C)c1ccc(N(c2ccc(C(C)C)cc2)c2ccc3cc4c(cc3c2)C(C)(C)c2c-4ccc3sc4cc5cc(N(c6ccc(C(C)C)cc6)c6ccc(C(C)C)cc6)ccc5cc4c23)cc1 ; CC1(C)c2cc3cc(N(c4ccccc4)c4ccc(C5CCCCC5)cc4)ccc3cc2-c2ccc3sc4cc5cc(N(c6ccccc6)c6ccc(C7CCCCC7)cc6)ccc5cc4c3c21]

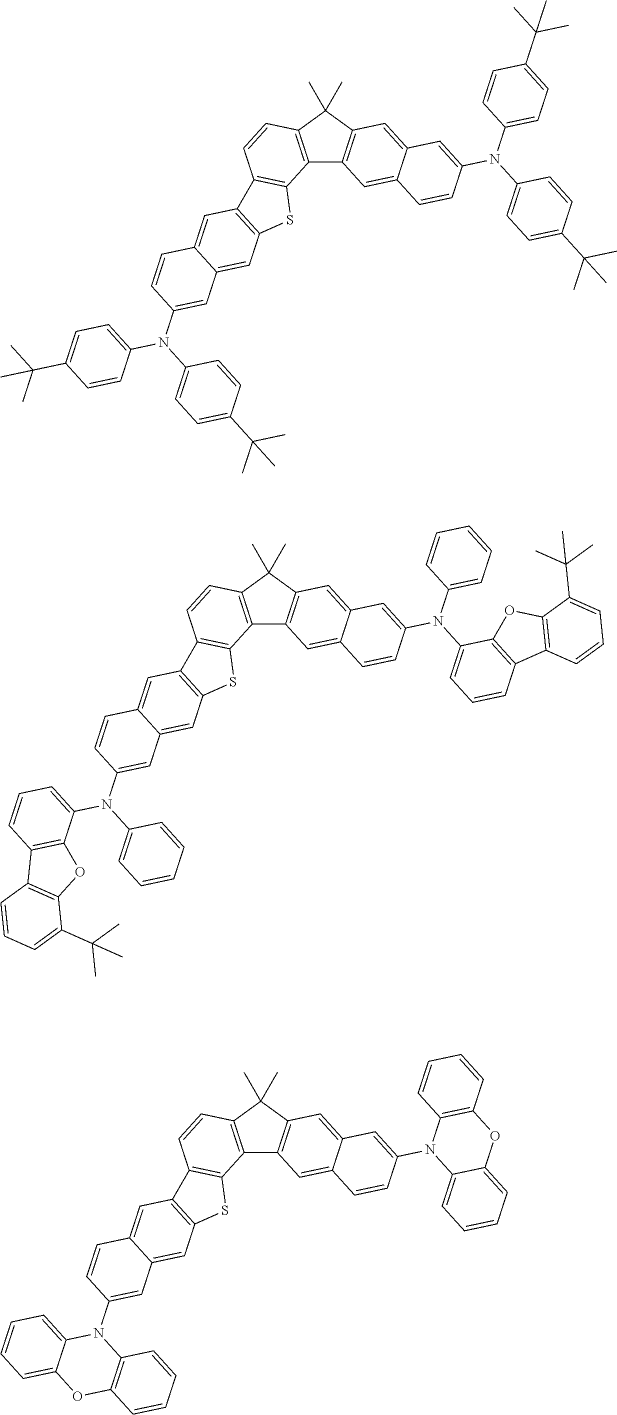 [SMILES: CC(C)(C)c1ccc(N(c2ccc(C(C)(C)C)cc2)c2ccc3cc4c(cc3c2)C(C)(C)c2ccc3c(sc5cc6cc(N(c7ccc(C(C)(C)C)cc7)c7ccc(C(C)(C)C)cc7)ccc6cc53)c2-4)cc1 ; CC(C)(C)c1cccc2c1oc1c(N(c3ccccc3)c3ccc4cc5c(cc4c3)C(C)(C)c3ccc4c(sc6cc7cc(N(c8ccccc8)c8cccc9c8oc8c(C(C)(C)C)cccc89)ccc7cc64)c3-5)cccc12 ; CC1(C)c2cc3cc(N4c5ccccc5Oc5ccccc54)ccc3cc2-c2c1ccc1c2sc2cc3cc(N4c5ccccc5Oc5ccccc54)ccc3cc21]

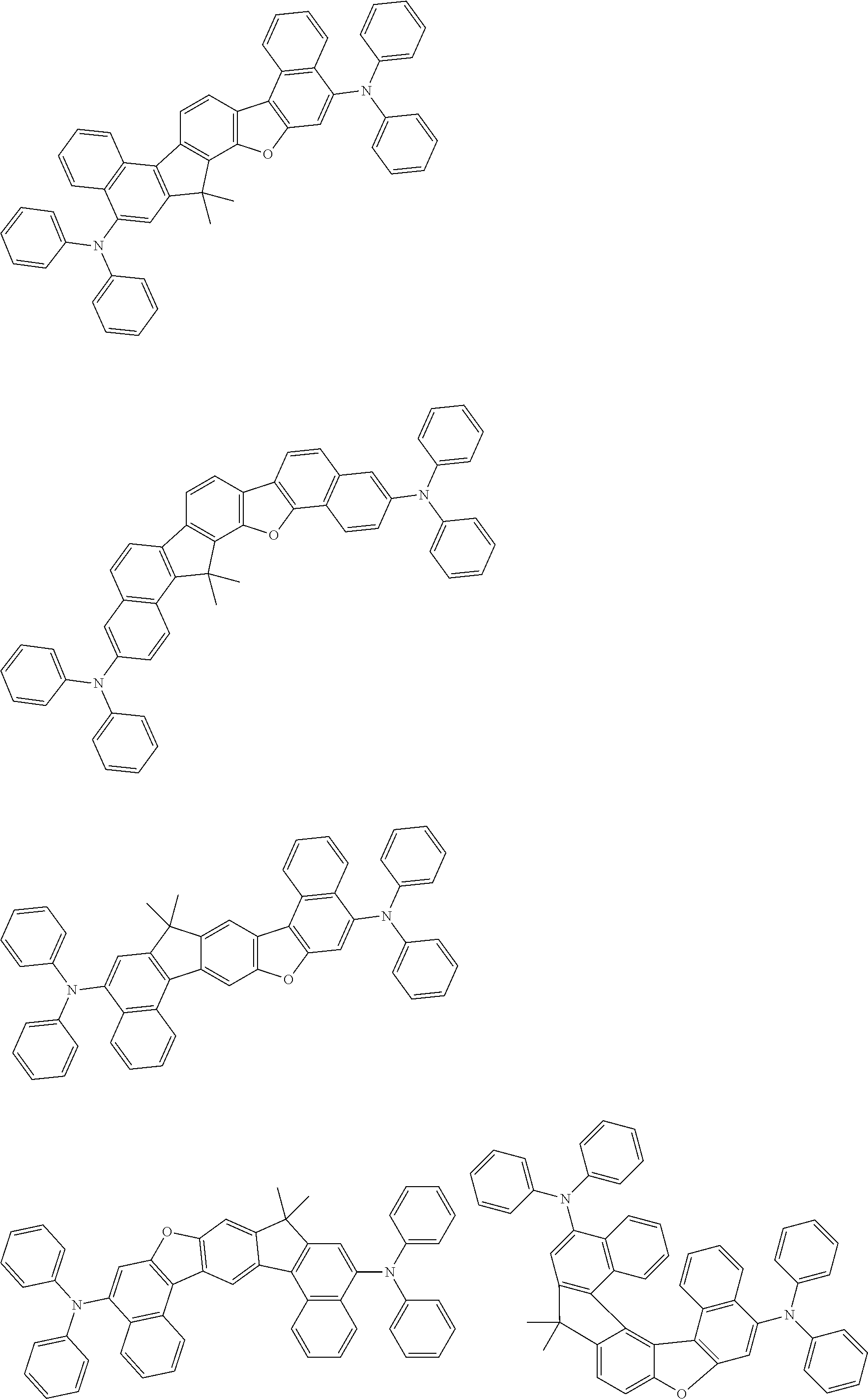

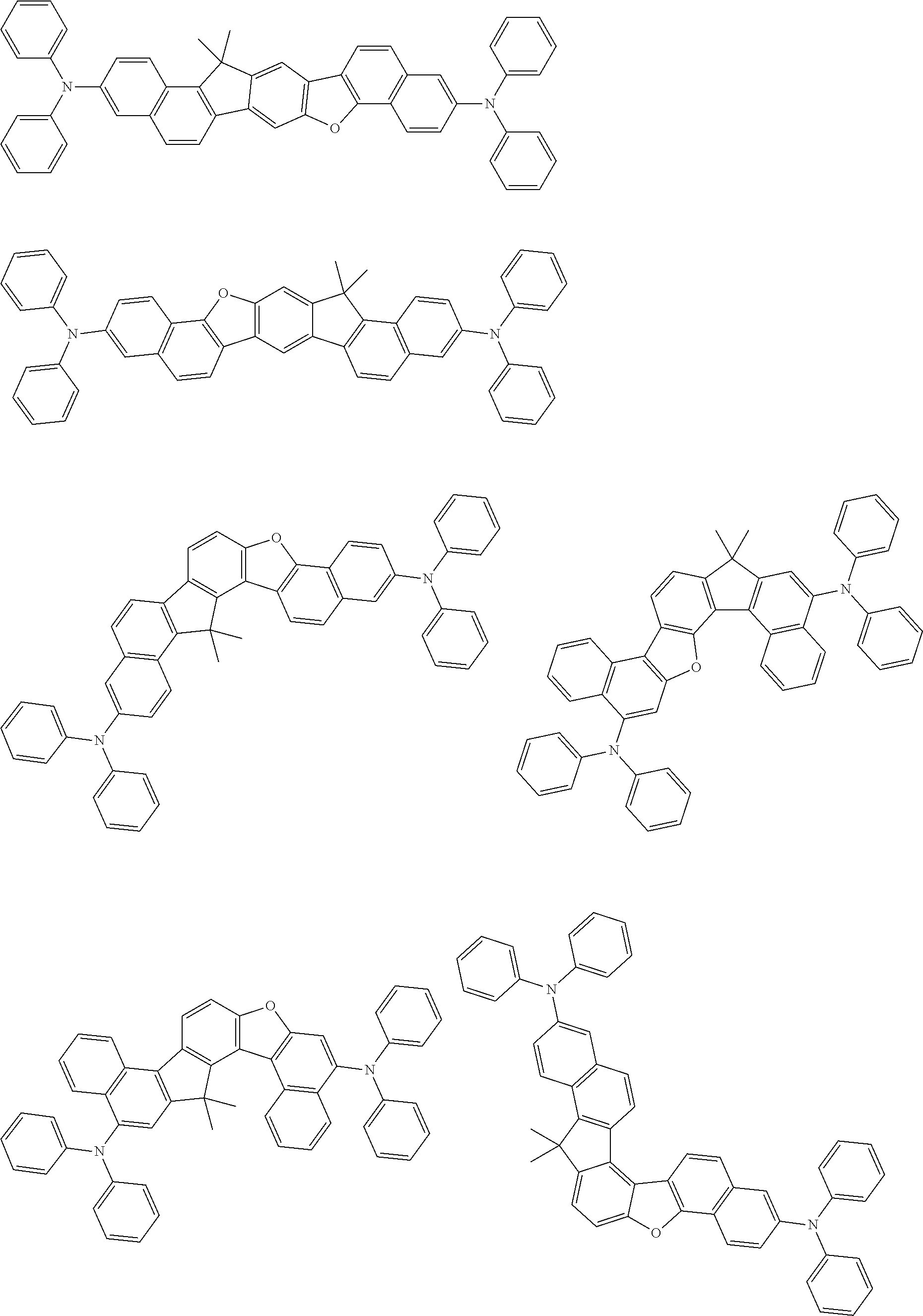

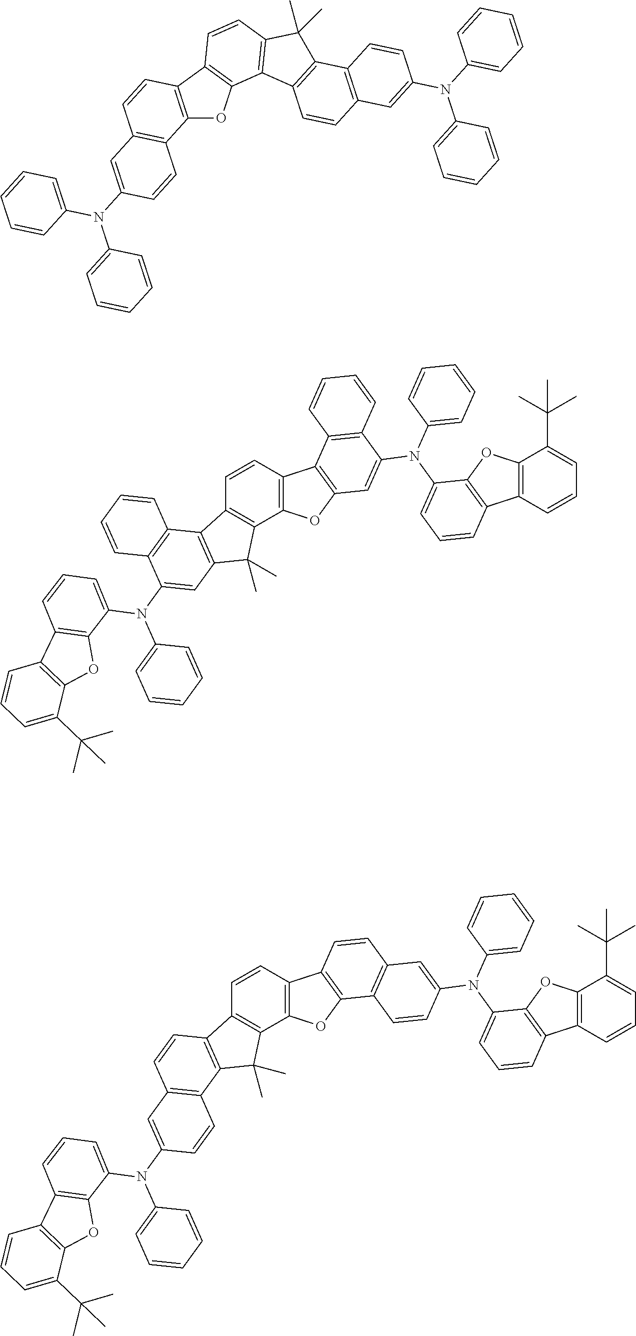 [SMILES: CC1(C)c2ccc3c(oc4c5ccc(N(c6ccccc6)c6ccccc6)cc5ccc34)c2-c2ccc3cc(N(c4ccccc4)c4ccccc4)ccc3c21 ; CC(C)(C)c1cccc2c1oc1c(N(c3ccccc3)c3cc4c(c5ccccc35)-c3ccc5c(oc6cc(N(c7ccccc7)c7cccc8c7oc7c(C(C)(C)C)cccc78)c7ccccc7c65)c3C4(C)C)cccc12 ; CC(C)(C)c1cccc2c1oc1c(N(c3ccccc3)c3ccc4c5c(ccc4c3)-c3ccc4c(oc6c7ccc(N(c8ccccc8)c8cccc9c8oc8c(C(C)(C)C)cccc89)cc7ccc46)c3C5(C)C)cccc12]

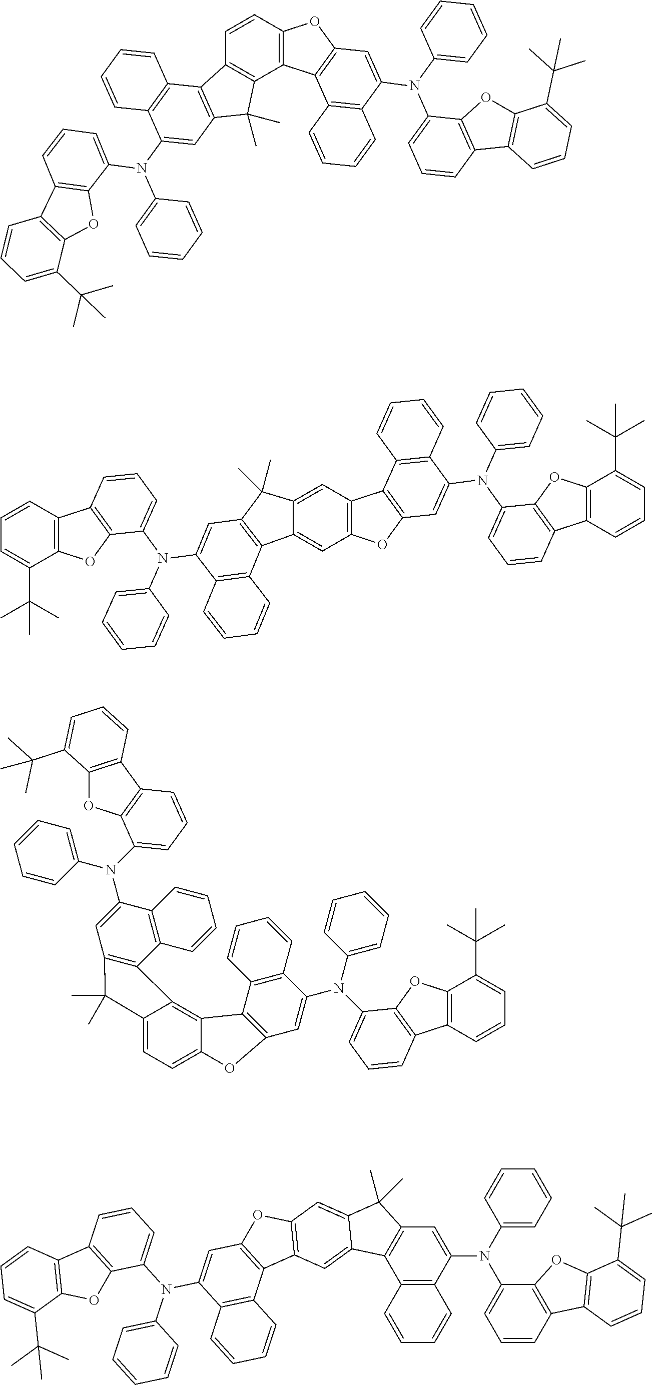

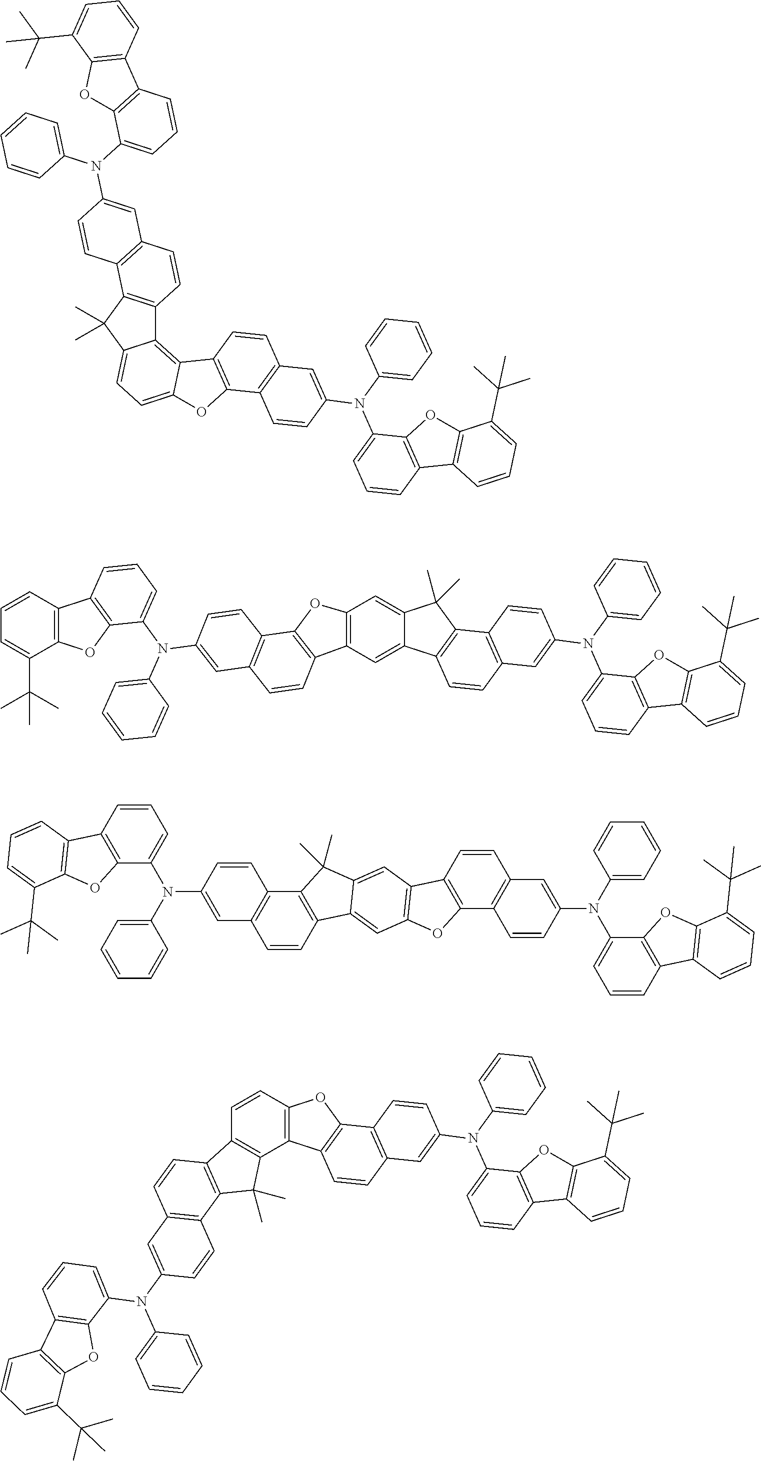

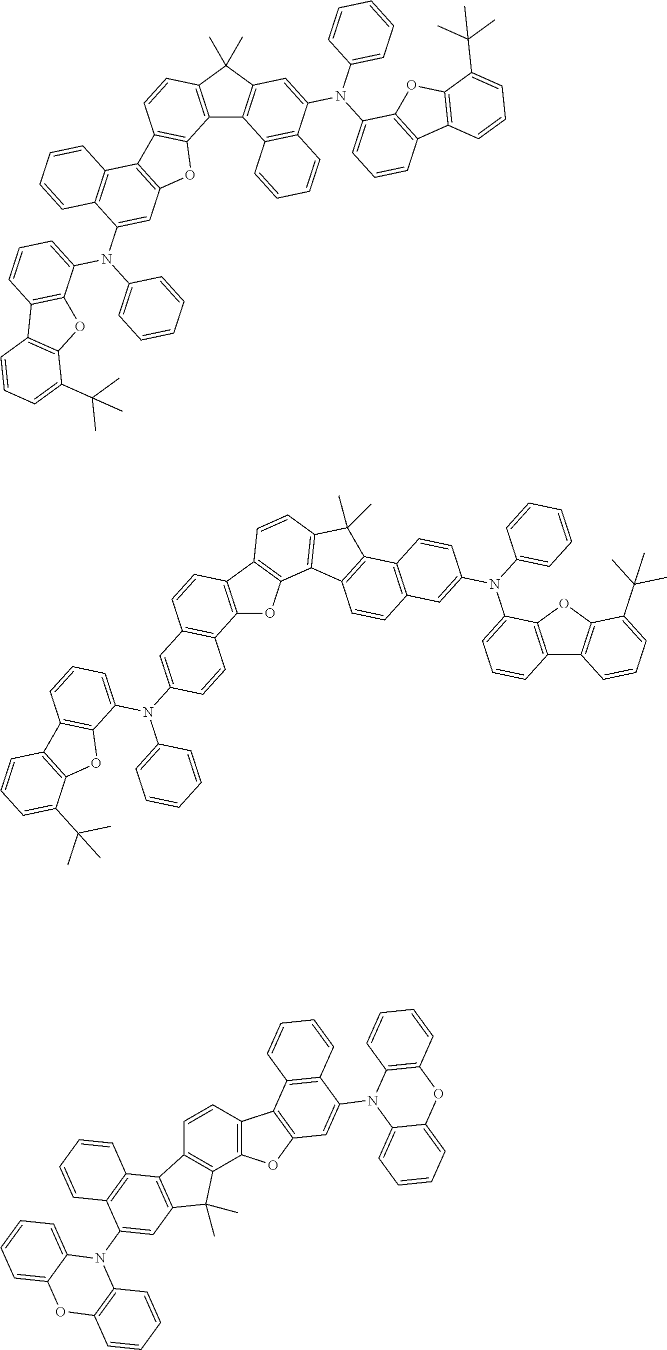 [SMILES: CC(C)(C)c1cccc2c1oc1c(N(c3ccccc3)c3cc4c(c5ccccc35)-c3c(ccc5c3oc3cc(N(c6ccccc6)c6cccc7c6oc6c(C(C)(C)C)cccc67)c6ccccc6c35)C4(C)C)cccc12 ; CC(C)(C)c1cccc2c1oc1c(N(c3ccccc3)c3ccc4c5c(ccc4c3)-c3c(ccc4c3oc3c6ccc(N(c7ccccc7)c7cccc8c7oc7c(C(C)(C)C)cccc78)cc6ccc43)C5(C)C)cccc12 ; CC1(C)c2cc(N3c4ccccc4Oc4ccccc43)c3ccccc3c2-c2ccc3c(oc4cc(N5c6ccccc6Oc6ccccc65)c5ccccc5c43)c21]

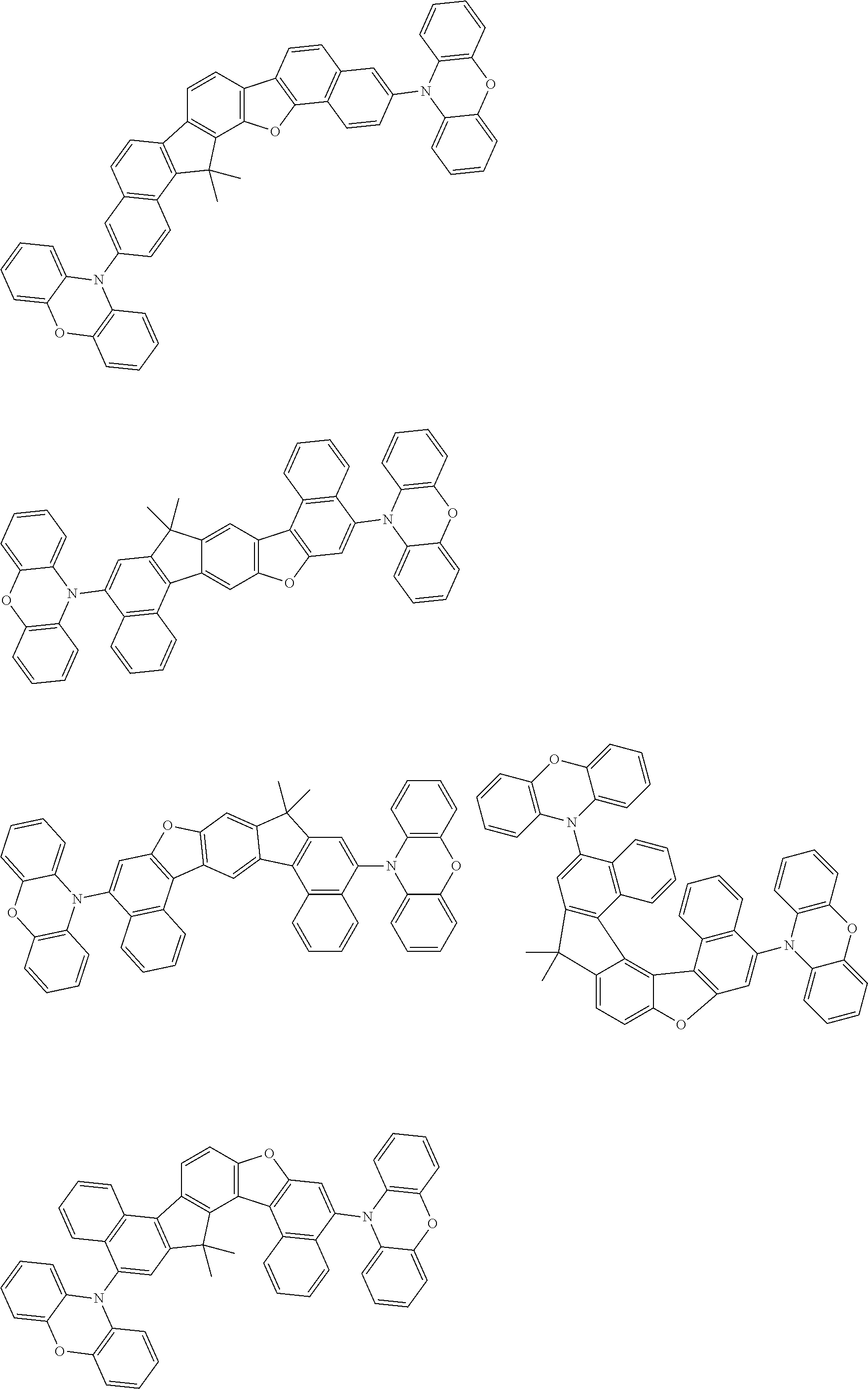

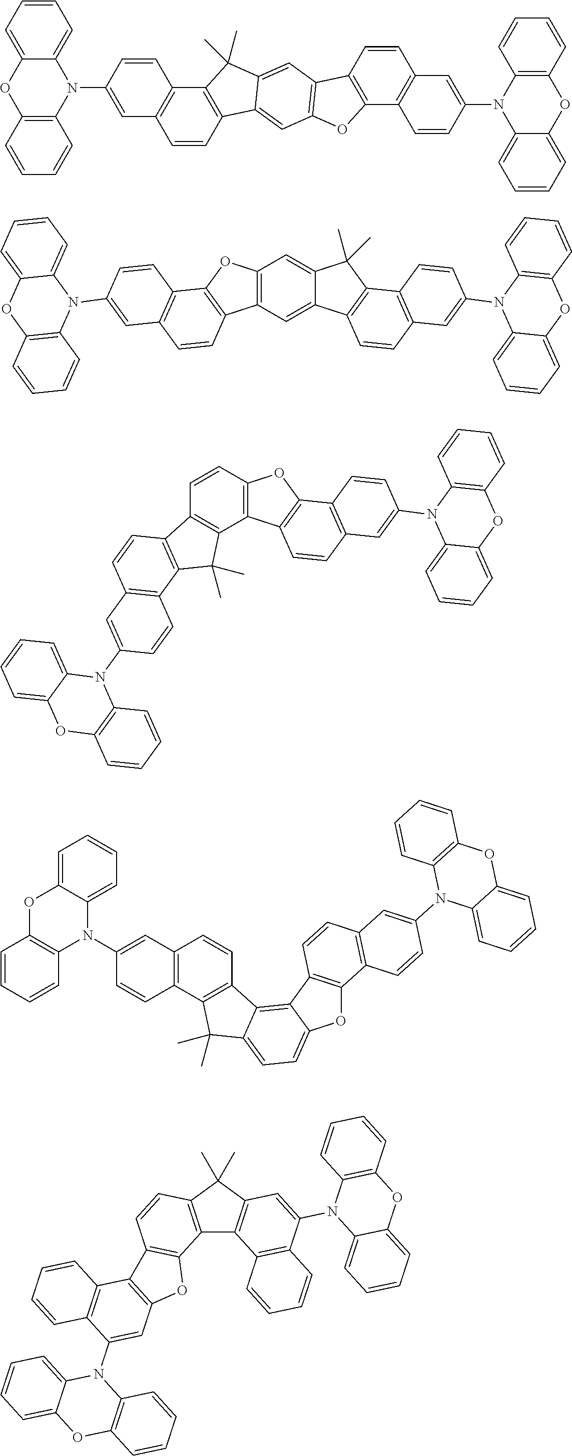

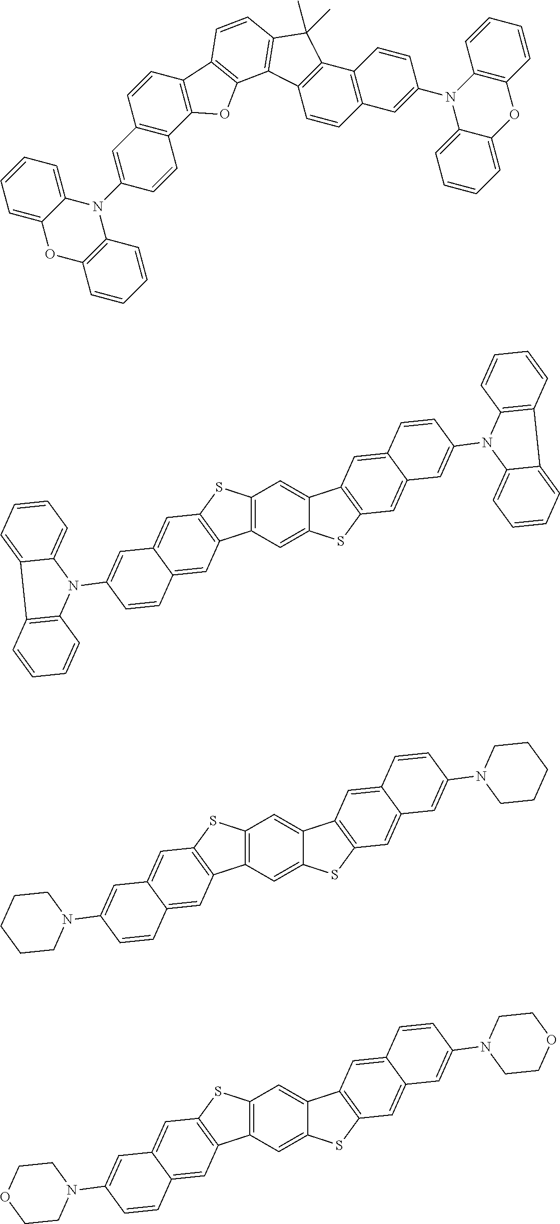 [SMILES: CC1(C)c2ccc3c(oc4c5ccc(N6c7ccccc7Oc7ccccc76)cc5ccc34)c2-c2ccc3cc(N4c5ccccc5Oc5ccccc54)ccc3c21 ; c1ccc2c(c1)c1ccccc1n2-c1ccc2cc3c(cc2c1)sc1cc2c(cc13)sc1cc3cc(-n4c5ccccc5c5ccccc54)ccc3cc12 ; c1cc2cc3c(cc2cc1N1CCCCC1)sc1cc2c(cc13)sc1cc3cc(N4CCCCC4)ccc3cc12 ; c1cc2cc3c(cc2cc1N1CCOCC1)sc1cc2c(cc13)sc1cc3cc(N4CCOCC4)ccc3cc12]

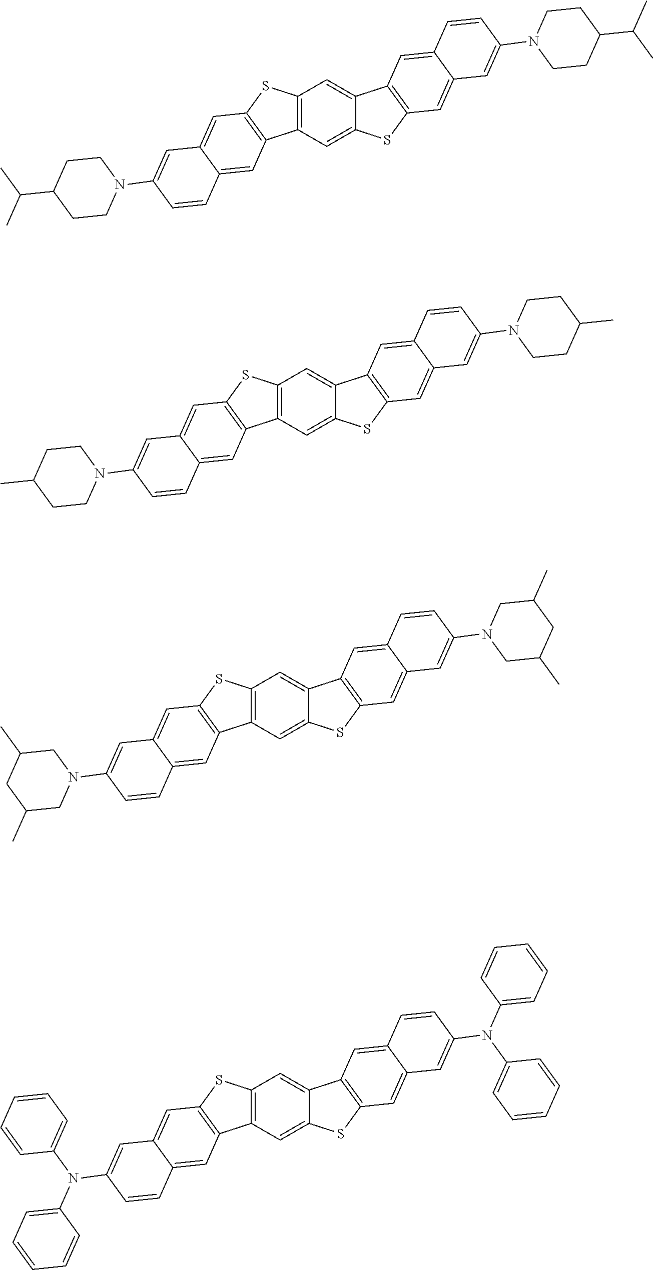

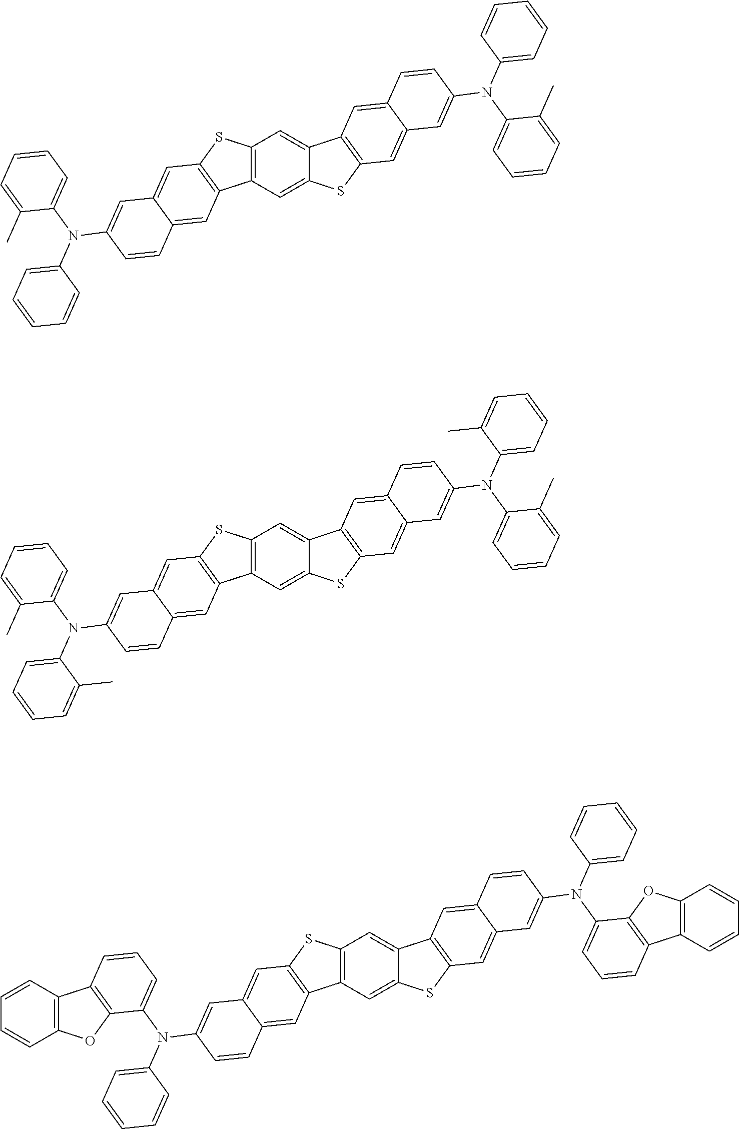 [SMILES: Cc1ccccc1N(c1ccccc1)c1ccc2cc3c(cc2c1)sc1cc2c(cc13)sc1cc3cc(N(c4ccccc4)c4ccccc4C)ccc3cc12 ; Cc1ccccc1N(c1ccc2cc3c(cc2c1)sc1cc2c(cc13)sc1cc3cc(N(c4ccccc4C)c4ccccc4C)ccc3cc12)c1ccccc1C ; c1ccc(N(c2ccc3cc4c(cc3c2)sc2cc3c(cc24)sc2cc4cc(N(c5ccccc5)c5cccc6c5oc5ccccc56)ccc4cc23)c2cccc3c2oc2ccccc23)cc1]

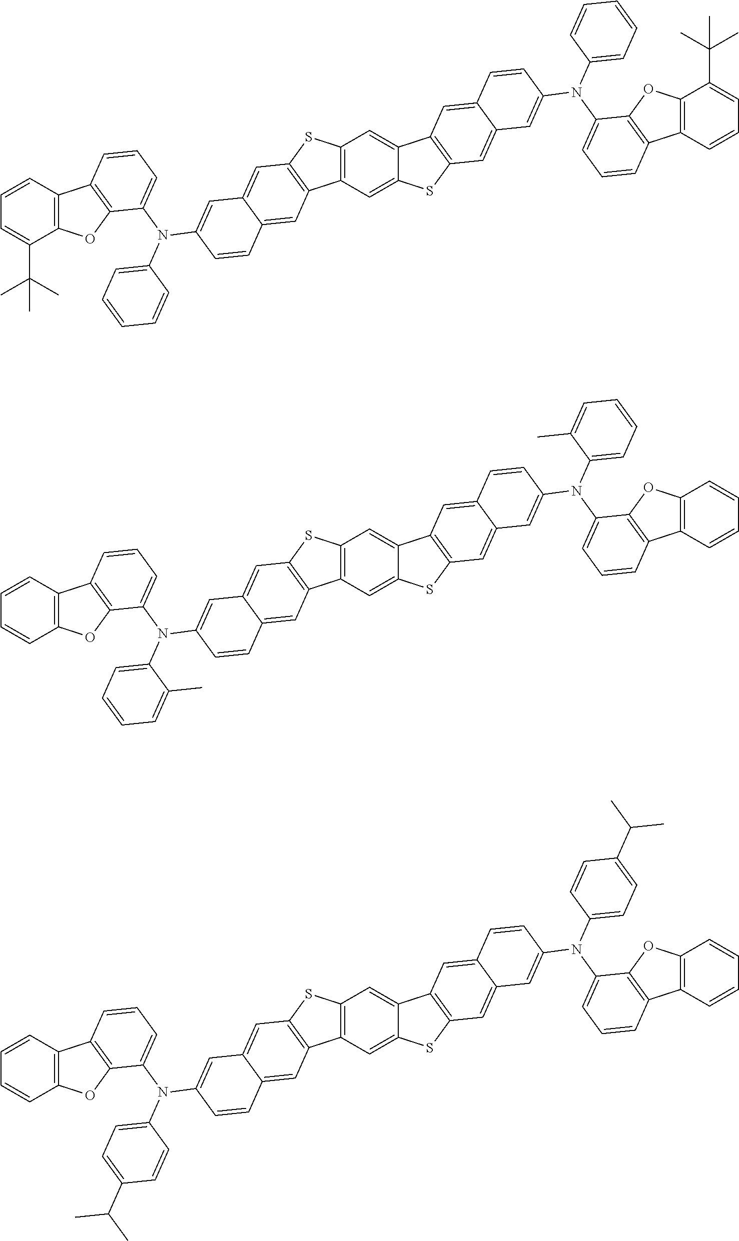 [SMILES: CC(C)(C)c1cccc2c1oc1c(N(c3ccccc3)c3ccc4cc5c(cc4c3)sc3cc4c(cc35)sc3cc5cc(N(c6ccccc6)c6cccc7c6oc6c(C(C)(C)C)cccc67)ccc5cc34)cccc12 ; Cc1ccccc1N(c1ccc2cc3c(cc2c1)sc1cc2c(cc13)sc1cc3cc(N(c4ccccc4C)c4cccc5c4oc4ccccc45)ccc3cc12)c1cccc2c1oc1ccccc12 ; CC(C)c1ccc(N(c2ccc3cc4c(cc3c2)sc2cc3c(cc24)sc2cc4cc(N(c5ccc(C(C)C)cc5)c5cccc6c5oc5ccccc56)ccc4cc23)c2cccc3c2oc2ccccc23)cc1]

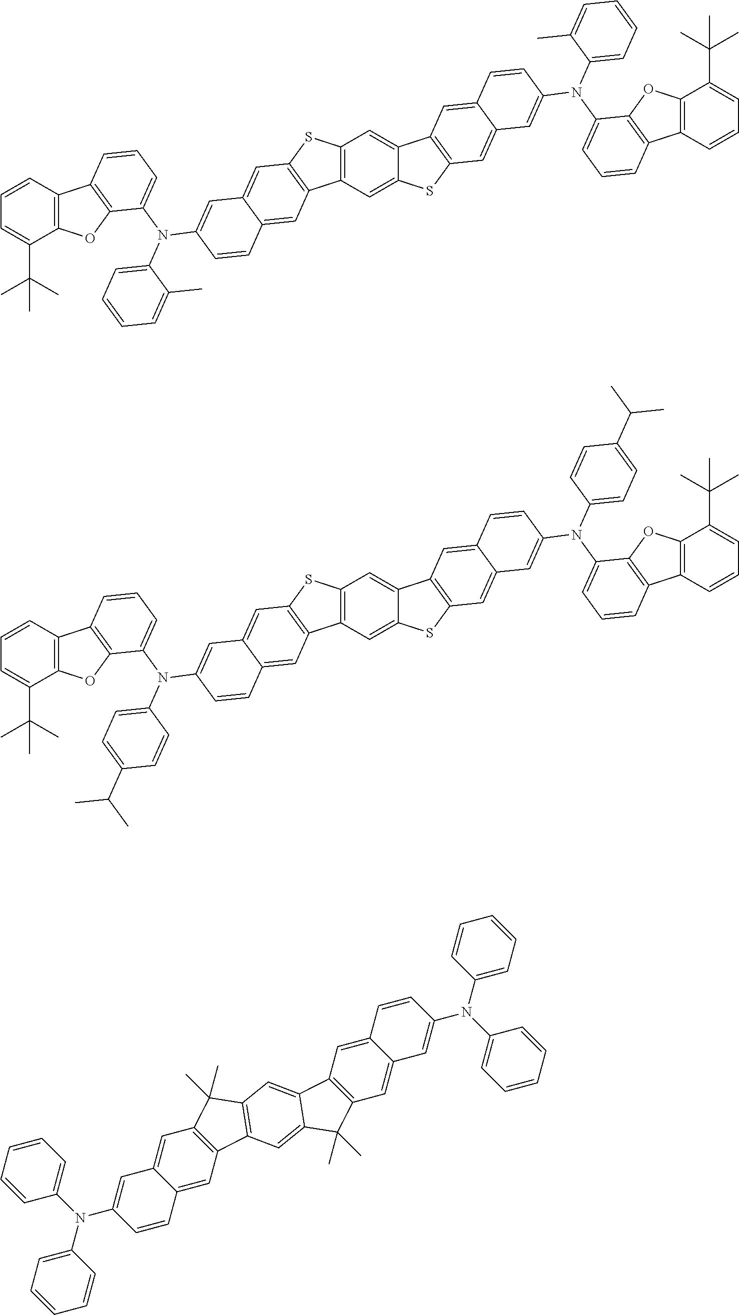 [SMILES: Cc1ccccc1N(c1ccc2cc3c(cc2c1)sc1cc2c(cc13)sc1cc3cc(N(c4ccccc4C)c4cccc5c4oc4c(C(C)(C)C)cccc45)ccc3cc12)c1cccc2c1oc1c(C(C)(C)C)cccc12 ; CC(C)c1ccc(N(c2ccc3cc4c(cc3c2)sc2cc3c(cc24)sc2cc4cc(N(c5ccc(C(C)C)cc5)c5cccc6c5oc5c(C(C)(C)C)cccc56)ccc4cc23)c2cccc3c2oc2c(C(C)(C)C)cccc23)cc1 ; CC1(C)c2cc3c(cc2-c2cc4ccc(N(c5ccccc5)c5ccccc5)cc4cc21)C(C)(C)c1cc2cc(N(c4ccccc4)c4ccccc4)ccc2cc1-3]

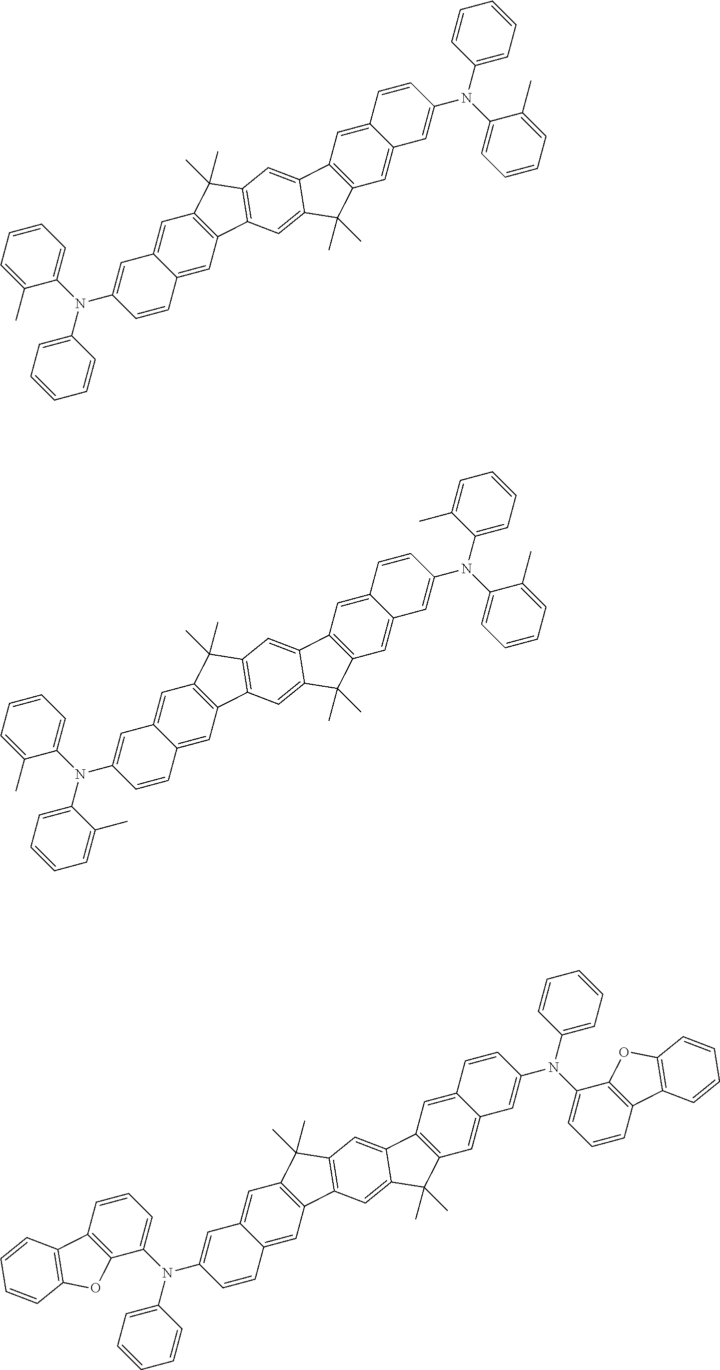 [SMILES: Cc1ccccc1N(c1ccccc1)c1ccc2cc3c(cc2c1)C(C)(C)c1cc2c(cc1-3)C(C)(C)c1cc3cc(N(c4ccccc4)c4ccccc4C)ccc3cc1-2 ; Cc1ccccc1N(c1ccc2cc3c(cc2c1)C(C)(C)c1cc2c(cc1-3)C(C)(C)c1cc3cc(N(c4ccccc4C)c4ccccc4C)ccc3cc1-2)c1ccccc1C ; CC1(C)c2cc3c(cc2-c2cc4ccc(N(c5ccccc5)c5cccc6c5oc5ccccc56)cc4cc21)C(C)(C)c1cc2cc(N(c4ccccc4)c4cccc5c4oc4ccccc45)ccc2cc1-3]

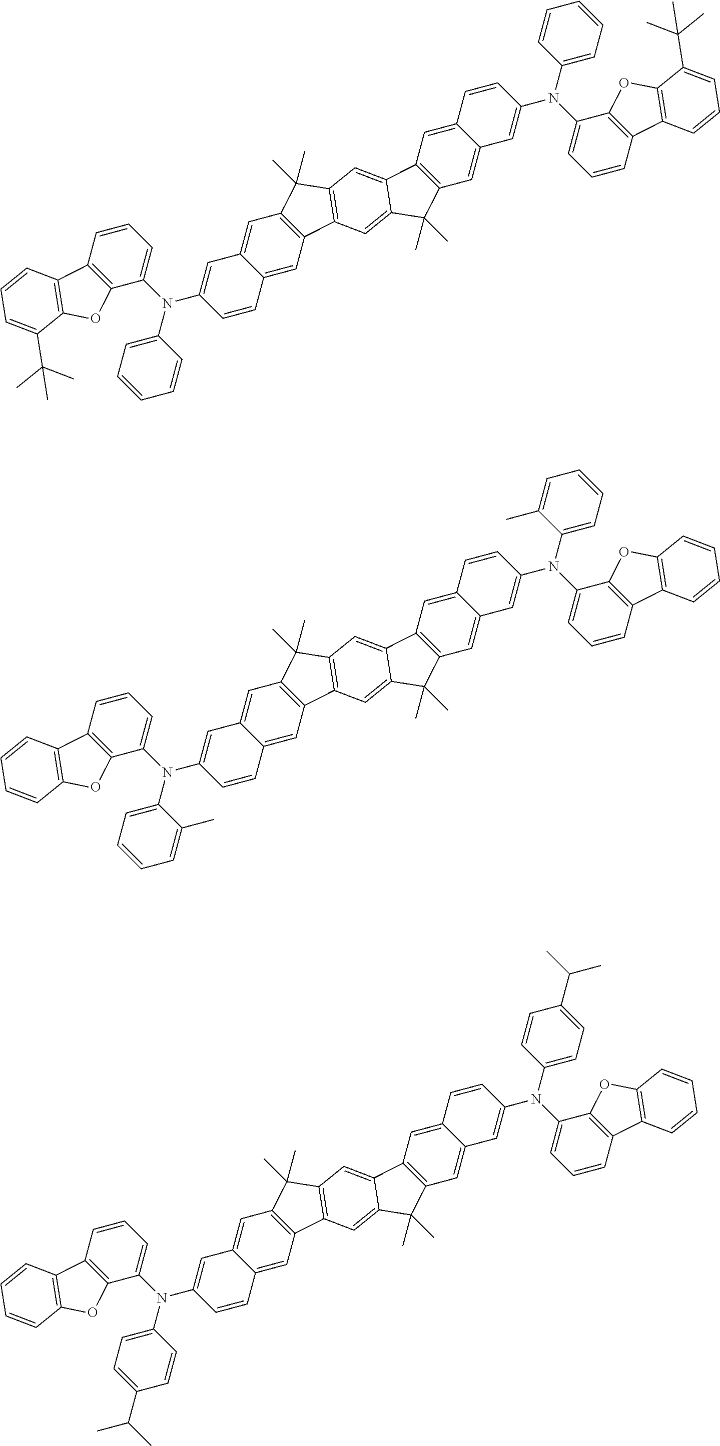 [SMILES: CC(C)(C)c1cccc2c1oc1c(N(c3ccccc3)c3ccc4cc5c(cc4c3)C(C)(C)c3cc4c(cc3-5)C(C)(C)c3cc5cc(N(c6ccccc6)c6cccc7c6oc6c(C(C)(C)C)cccc67)ccc5cc3-4)cccc12 ; Cc1ccccc1N(c1ccc2cc3c(cc2c1)C(C)(C)c1cc2c(cc1-3)C(C)(C)c1cc3cc(N(c4ccccc4C)c4cccc5c4oc4ccccc45)ccc3cc1-2)c1cccc2c1oc1ccccc12 ; CC(C)c1ccc(N(c2ccc3cc4c(cc3c2)C(C)(C)c2cc3c(cc2-4)C(C)(C)c2cc4cc(N(c5ccc(C(C)C)cc5)c5cccc6c5oc5ccccc56)ccc4cc2-3)c2cccc3c2oc2ccccc23)cc1]

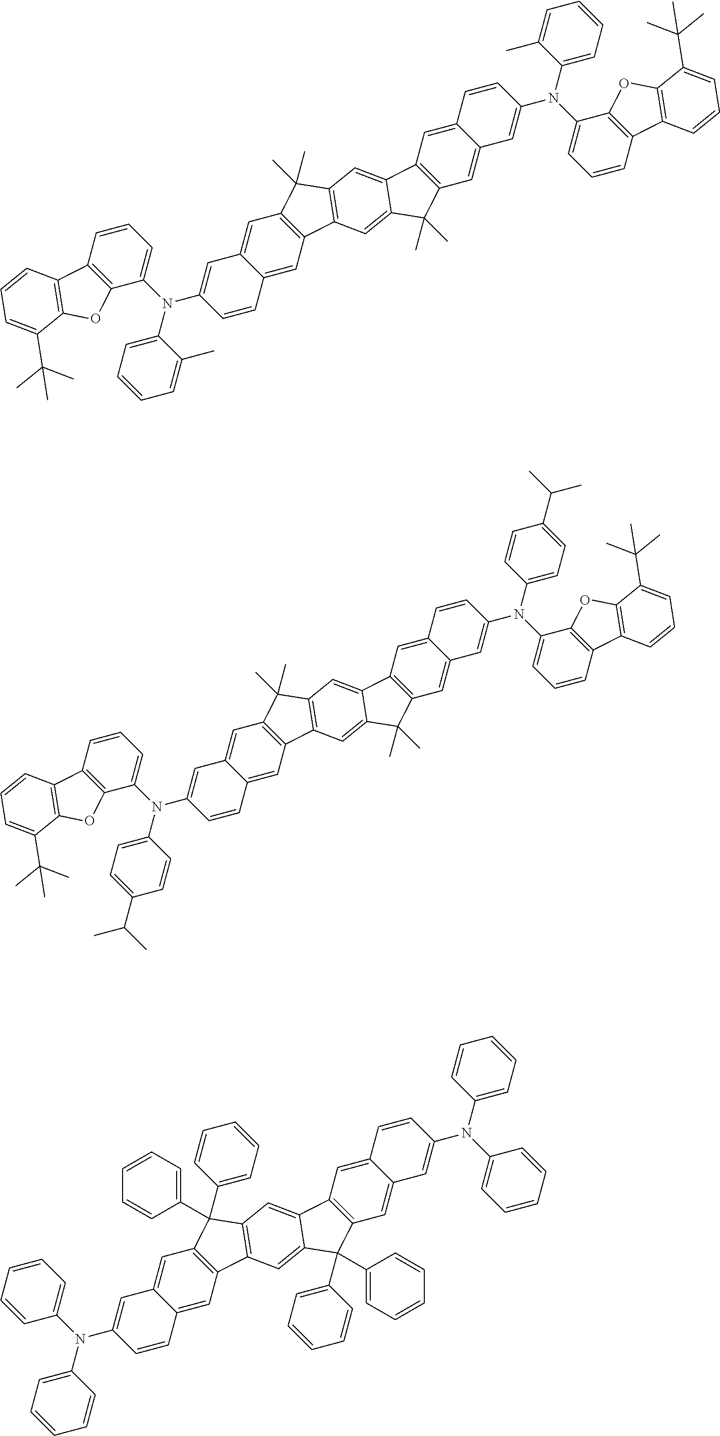 [SMILES: Cc1ccccc1N(c1ccc2cc3c(cc2c1)C(C)(C)c1cc2c(cc1-3)C(C)(C)c1cc3cc(N(c4ccccc4C)c4cccc5c4oc4c(C(C)(C)C)cccc45)ccc3cc1-2)c1cccc2c1oc1c(C(C)(C)C)cccc12 ; CC(C)c1ccc(N(c2ccc3cc4c(cc3c2)C(C)(C)c2cc3c(cc2-4)C(C)(C)c2cc4cc(N(c5ccc(C(C)C)cc5)c5cccc6c5oc5c(C(C)(C)C)cccc56)ccc4cc2-3)c2cccc3c2oc2c(C(C)(C)C)cccc23)cc1 ; c1ccc(N(c2ccccc2)c2ccc3cc4c(cc3c2)C(c2ccccc2)(c2ccccc2)c2cc3c(cc2-4)C(c2ccccc2)(c2ccccc2)c2cc4cc(N(c5ccccc5)c5ccccc5)ccc4cc2-3)cc1]

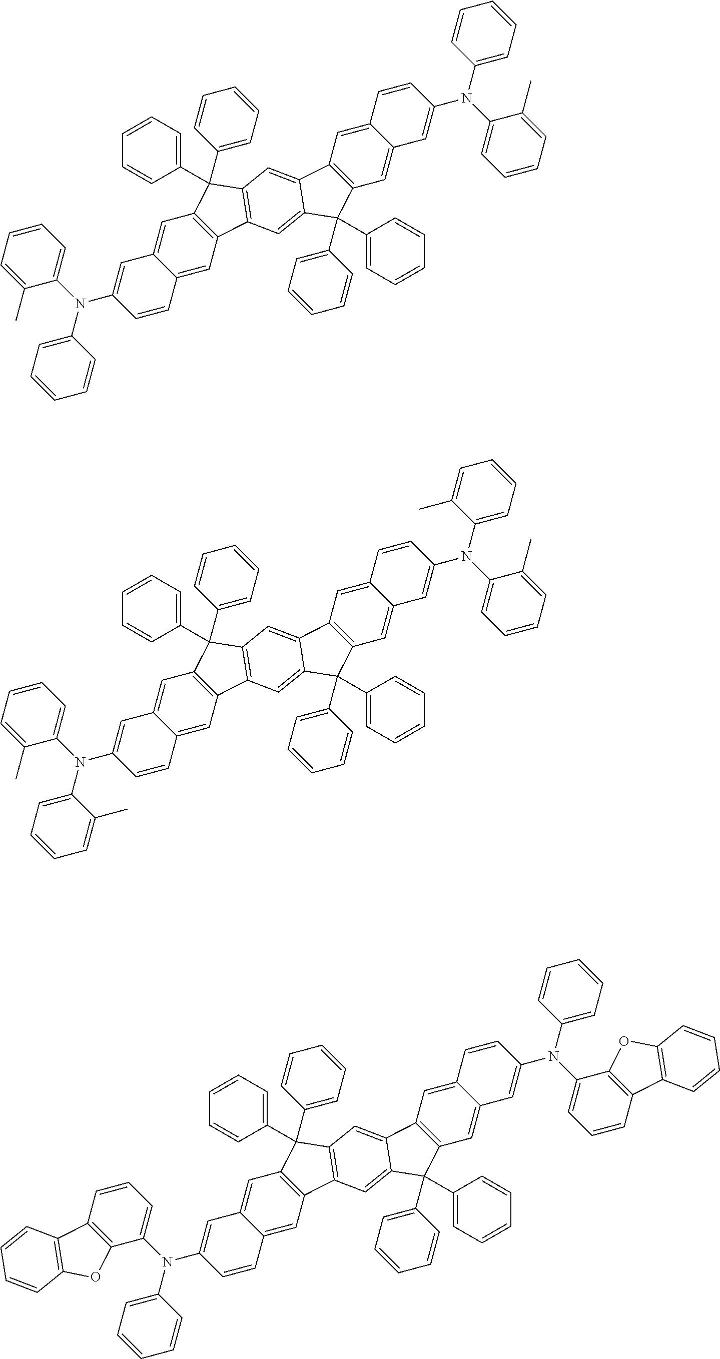 [SMILES: Cc1ccccc1N(c1ccccc1)c1ccc2cc3c(cc2c1)C(c1ccccc1)(c1ccccc1)c1cc2c(cc1-3)C(c1ccccc1)(c1ccccc1)c1cc3cc(N(c4ccccc4)c4ccccc4C)ccc3cc1-2 ; Cc1ccccc1N(c1ccc2cc3c(cc2c1)C(c1ccccc1)(c1ccccc1)c1cc2c(cc1-3)C(c1ccccc1)(c1ccccc1)c1cc3cc(N(c4ccccc4C)c4ccccc4C)ccc3cc1-2)c1ccccc1C ; c1ccc(N(c2ccc3cc4c(cc3c2)C(c2ccccc2)(c2ccccc2)c2cc3c(cc2-4)C(c2ccccc2)(c2ccccc2)c2cc4cc(N(c5ccccc5)c5cccc6c5oc5ccccc56)ccc4cc2-3)c2cccc3c2oc2ccccc23)cc1]

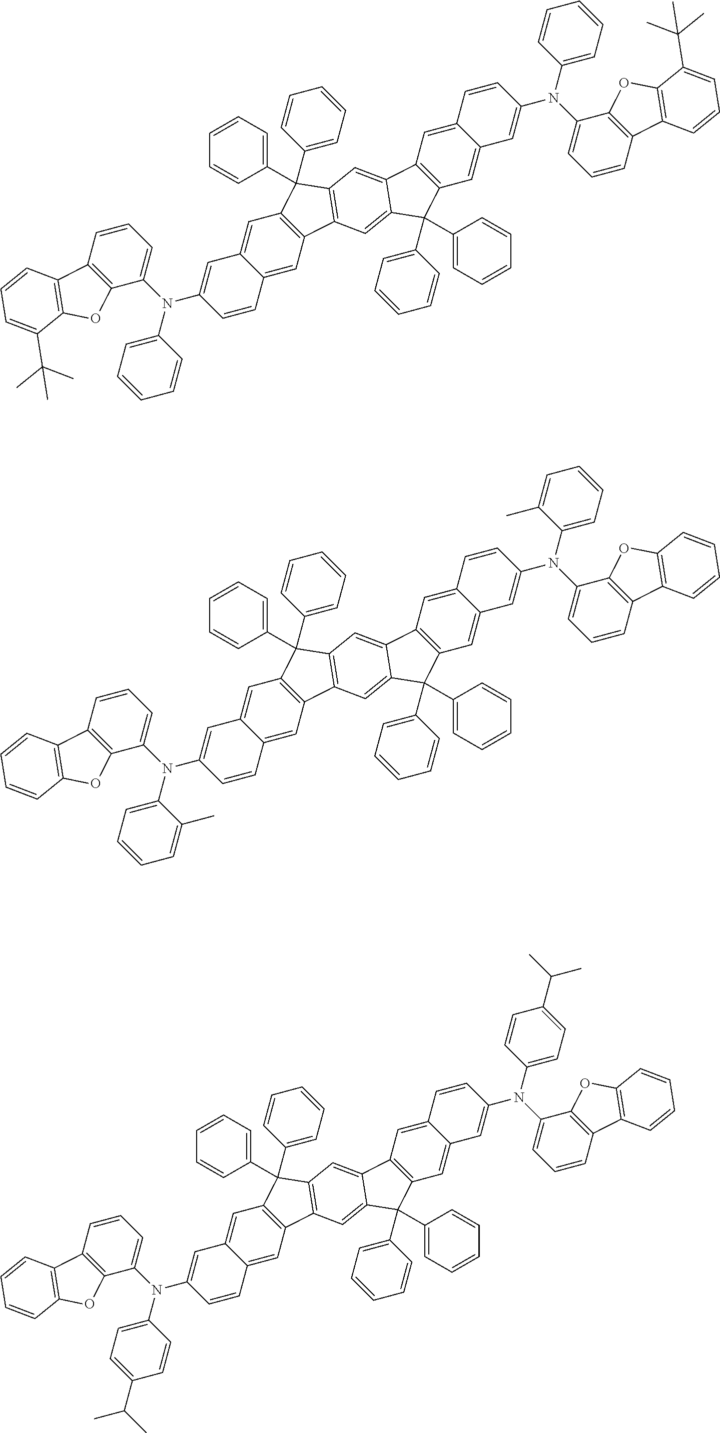 [SMILES: CC(C)(C)c1cccc2c1oc1c(N(c3ccccc3)c3ccc4cc5c(cc4c3)C(c3ccccc3)(c3ccccc3)c3cc4c(cc3-5)C(c3ccccc3)(c3ccccc3)c3cc5cc(N(c6ccccc6)c6cccc7c6oc6c(C(C)(C)C)cccc67)ccc5cc3-4)cccc12 ; Cc1ccccc1N(c1ccc2cc3c(cc2c1)C(c1ccccc1)(c1ccccc1)c1cc2c(cc1-3)C(c1ccccc1)(c1ccccc1)c1cc3cc(N(c4ccccc4C)c4cccc5c4oc4ccccc45)ccc3cc1-2)c1cccc2c1oc1ccccc12 ; CC(C)c1ccc(N(c2ccc3cc4c(cc3c2)C(c2ccccc2)(c2ccccc2)c2cc3c(cc2-4)C(c2ccccc2)(c2ccccc2)c2cc4cc(N(c5ccc(C(C)C)cc5)c5cccc6c5oc5ccccc56)ccc4cc2-3)c2cccc3c2oc2ccccc23)cc1]

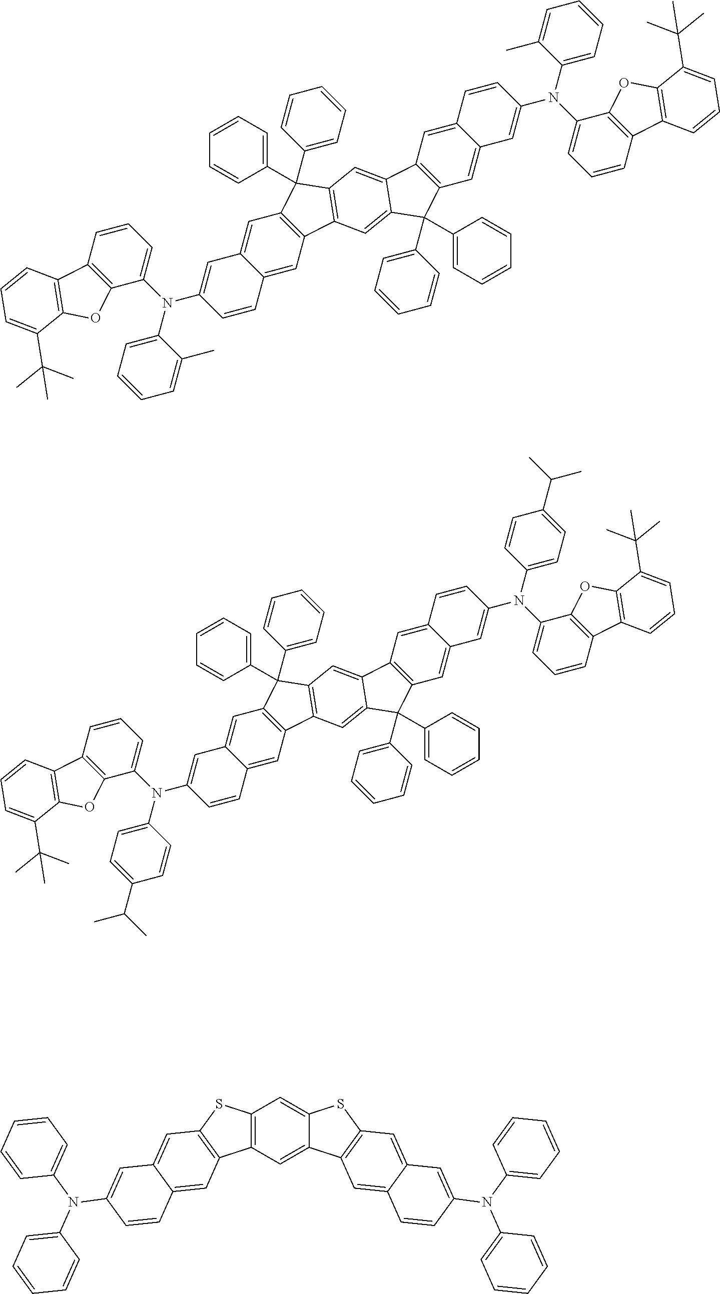 [SMILES: Cc1ccccc1N(c1ccc2cc3c(cc2c1)C(c1ccccc1)(c1ccccc1)c1cc2c(cc1-3)C(c1ccccc1)(c1ccccc1)c1cc3cc(N(c4ccccc4C)c4cccc5c4oc4c(C(C)(C)C)cccc45)ccc3cc1-2)c1cccc2c1oc1c(C(C)(C)C)cccc12 ; CC(C)c1ccc(N(c2ccc3cc4c(cc3c2)C(c2ccccc2)(c2ccccc2)c2cc3c(cc2-4)C(c2ccccc2)(c2ccccc2)c2cc4cc(N(c5ccc(C(C)C)cc5)c5cccc6c5oc5c(C(C)(C)C)cccc56)ccc4cc2-3)c2cccc3c2oc2c(C(C)(C)C)cccc23)cc1 ; c1ccc(N(c2ccccc2)c2ccc3cc4c(cc3c2)sc2cc3sc5cc6cc(N(c7ccccc7)c7ccccc7)ccc6cc5c3cc24)cc1]

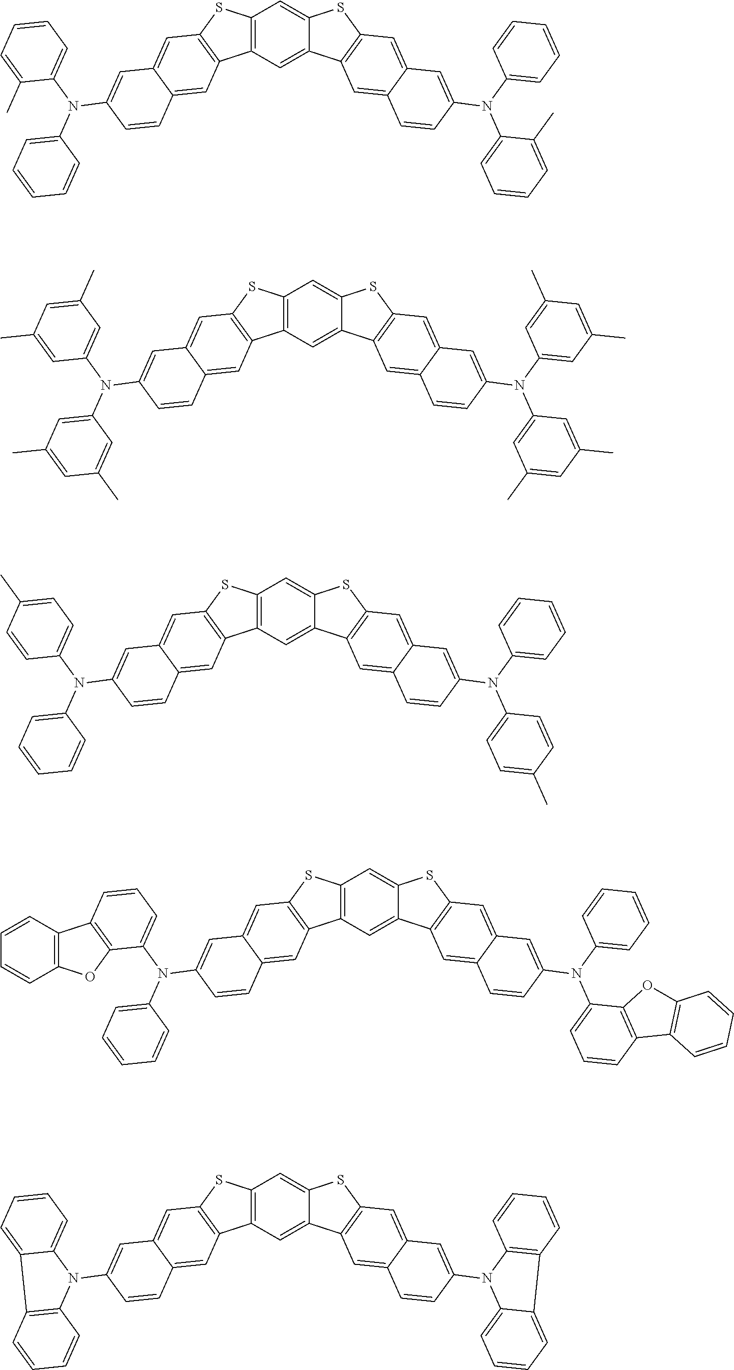

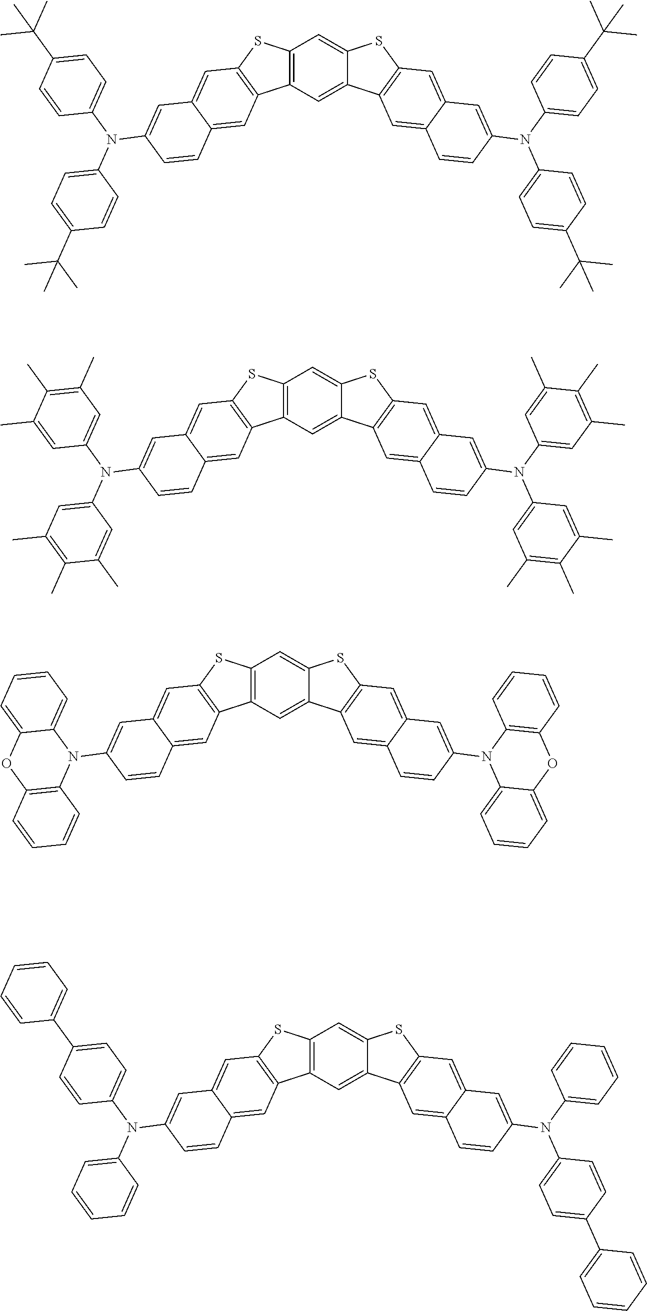

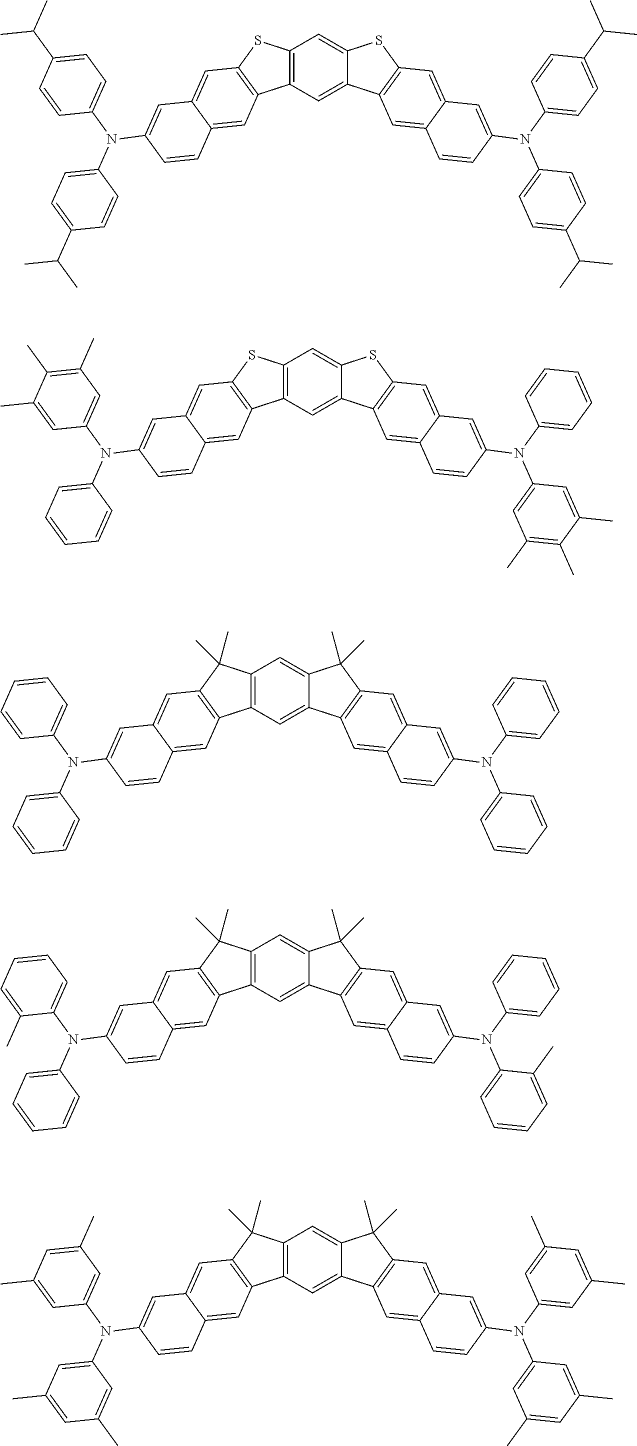

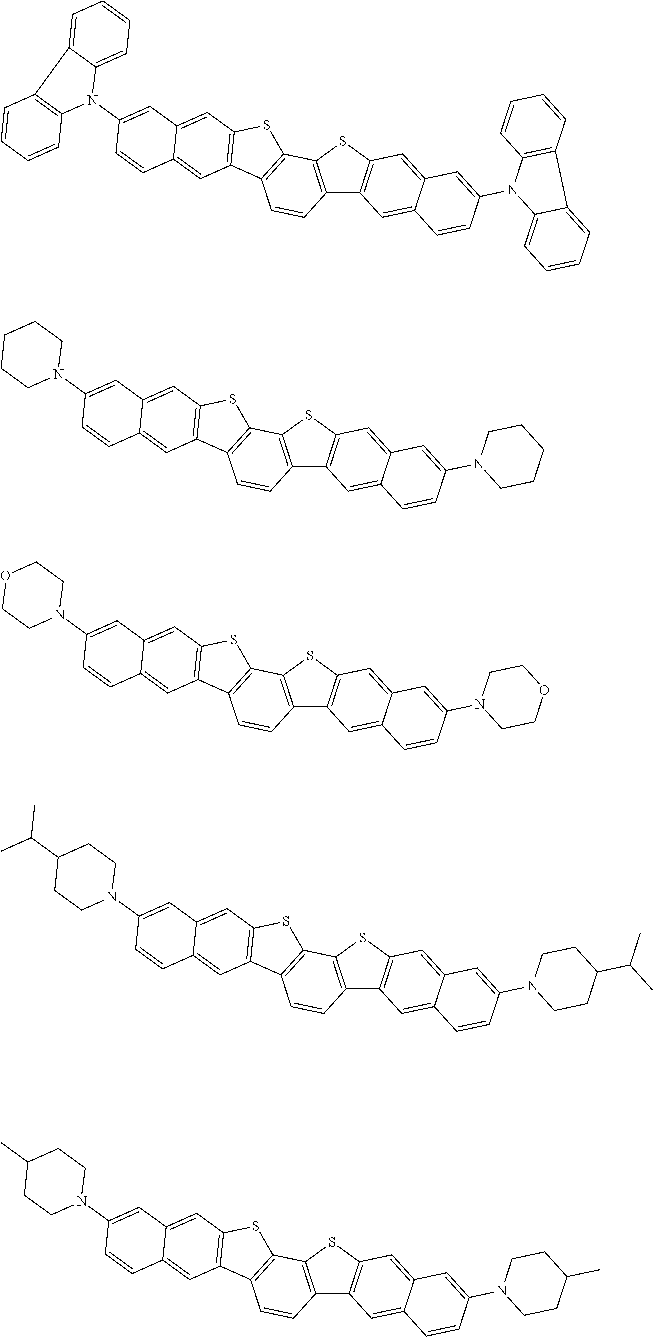

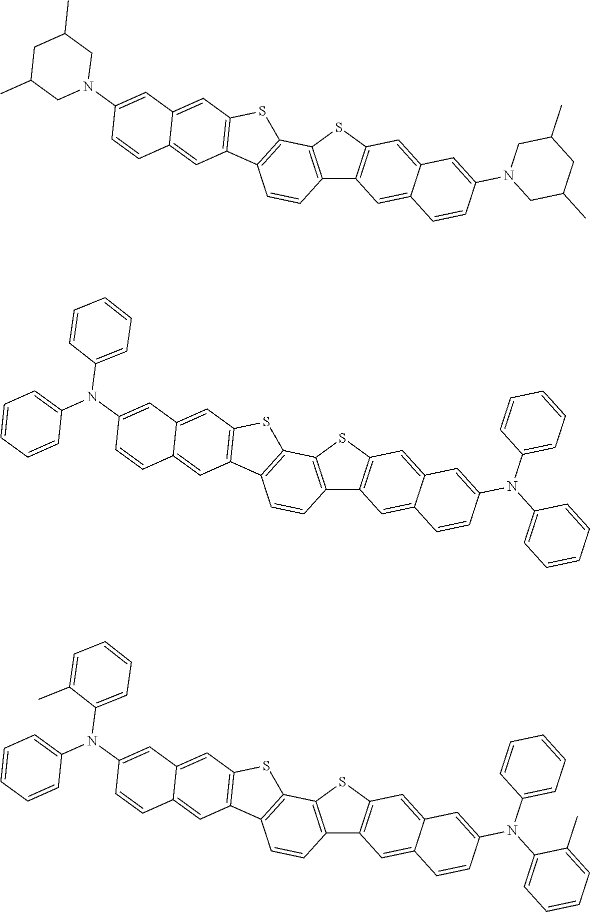

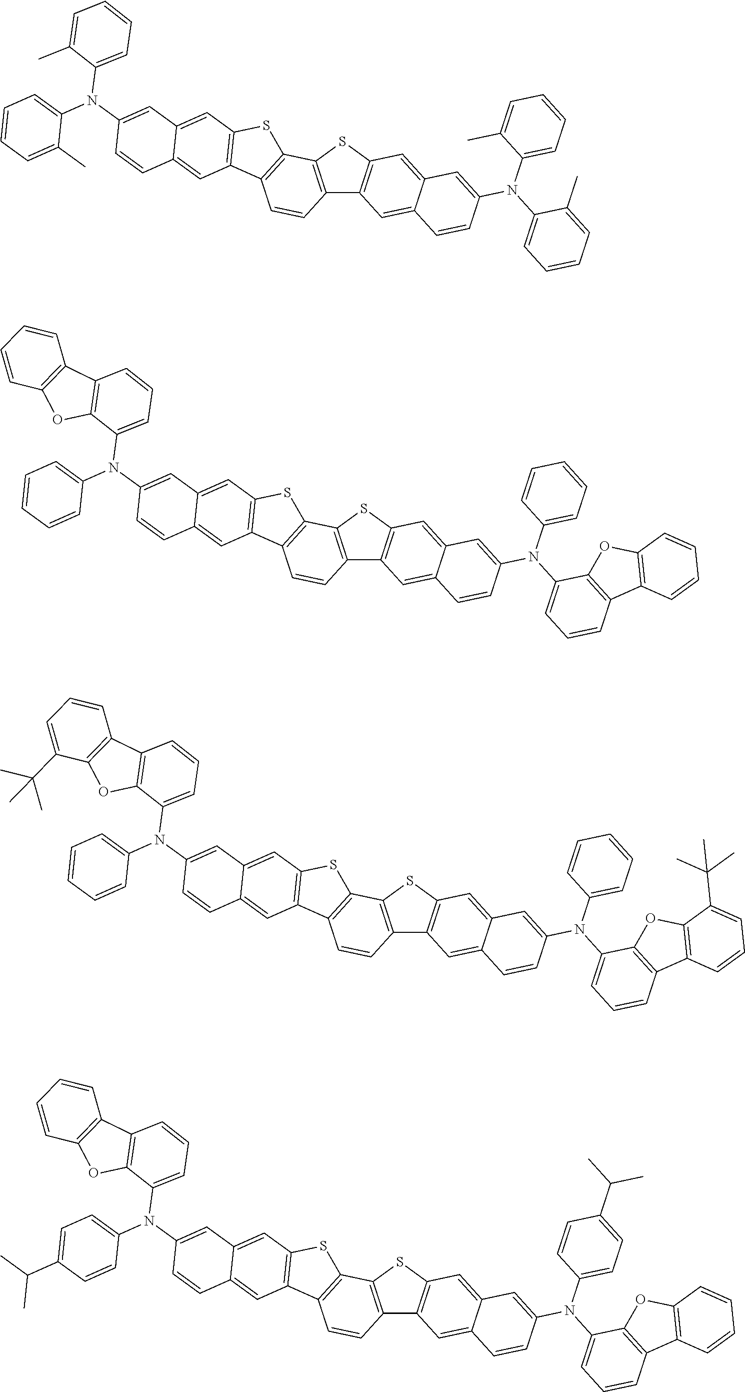 [SMILES: Cc1ccccc1N(c1ccc2cc3c(cc2c1)sc1c3ccc2c3cc4ccc(N(c5ccccc5C)c5ccccc5C)cc4cc3sc21)c1ccccc1C ; c1ccc(N(c2ccc3cc4c(cc3c2)sc2c4ccc3c4cc5ccc(N(c6ccccc6)c6cccc7c6oc6ccccc67)cc5cc4sc32)c2cccc3c2oc2ccccc23)cc1 ; CC(C)(C)c1cccc2c1oc1c(N(c3ccccc3)c3ccc4cc5c(cc4c3)sc3c5ccc4c5cc6ccc(N(c7ccccc7)c7cccc8c7oc7c(C(C)(C)C)cccc78)cc6cc5sc43)cccc12 ; CC(C)c1ccc(N(c2ccc3cc4c(cc3c2)sc2c4ccc3c4cc5ccc(N(c6ccc(C(C)C)cc6)c6cccc7c6oc6ccccc67)cc5cc4sc32)c2cccc3c2oc2ccccc23)cc1]

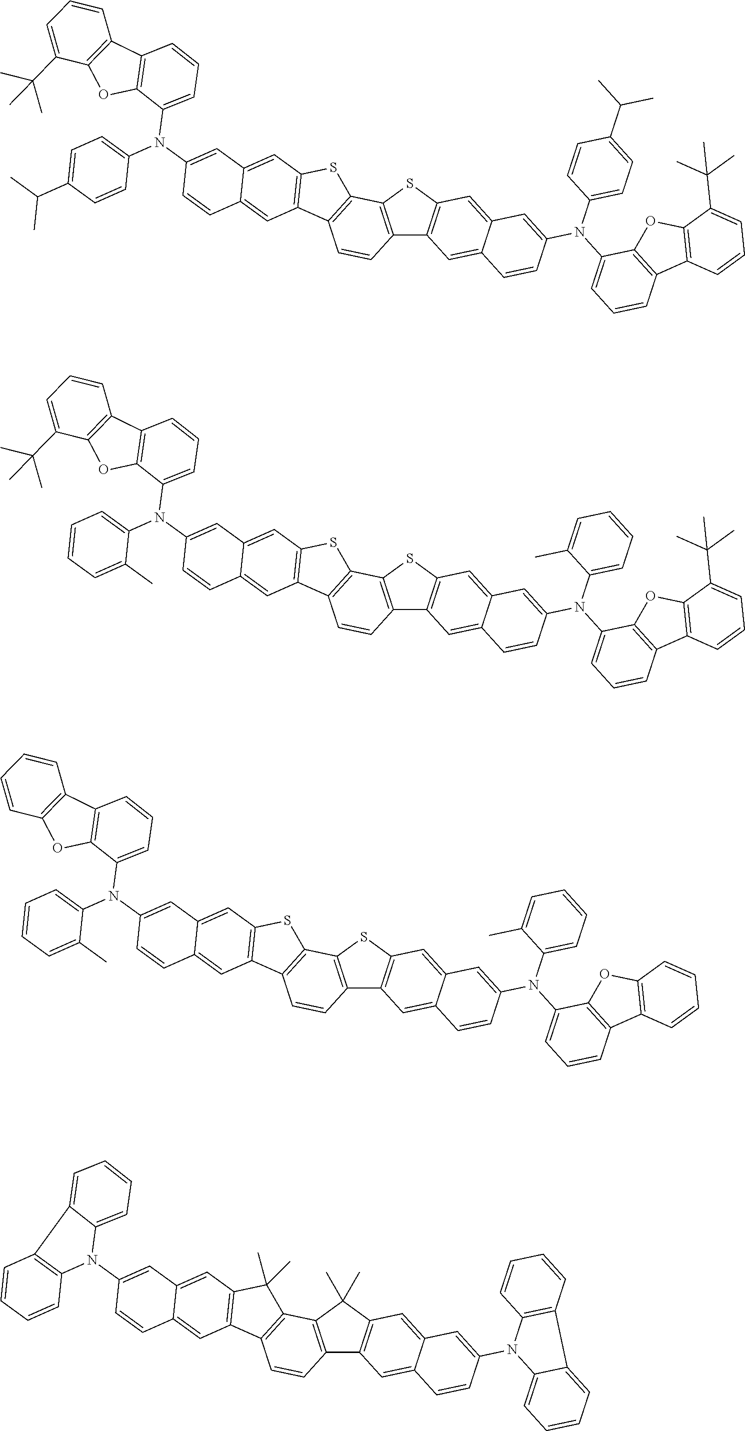 [SMILES: CC(C)c1ccc(N(c2ccc3cc4c(cc3c2)sc2c4ccc3c4cc5ccc(N(c6ccc(C(C)C)cc6)c6cccc7c6oc6c(C(C)(C)C)cccc67)cc5cc4sc32)c2cccc3c2oc2c(C(C)(C)C)cccc23)cc1 ; Cc1ccccc1N(c1ccc2cc3c(cc2c1)sc1c3ccc2c3cc4ccc(N(c5ccccc5C)c5cccc6c5oc5c(C(C)(C)C)cccc56)cc4cc3sc21)c1cccc2c1oc1c(C(C)(C)C)cccc12 ; Cc1ccccc1N(c1ccc2cc3c(cc2c1)sc1c3ccc2c3cc4ccc(N(c5ccccc5C)c5cccc6c5oc5ccccc56)cc4cc3sc21)c1cccc2c1oc1ccccc12 ; CC1(C)c2cc3cc(-n4c5ccccc5c5ccccc54)ccc3cc2-c2ccc3c(c21)C(C)(C)c1cc2cc(-n4c5ccccc5c5ccccc54)ccc2cc1-3]

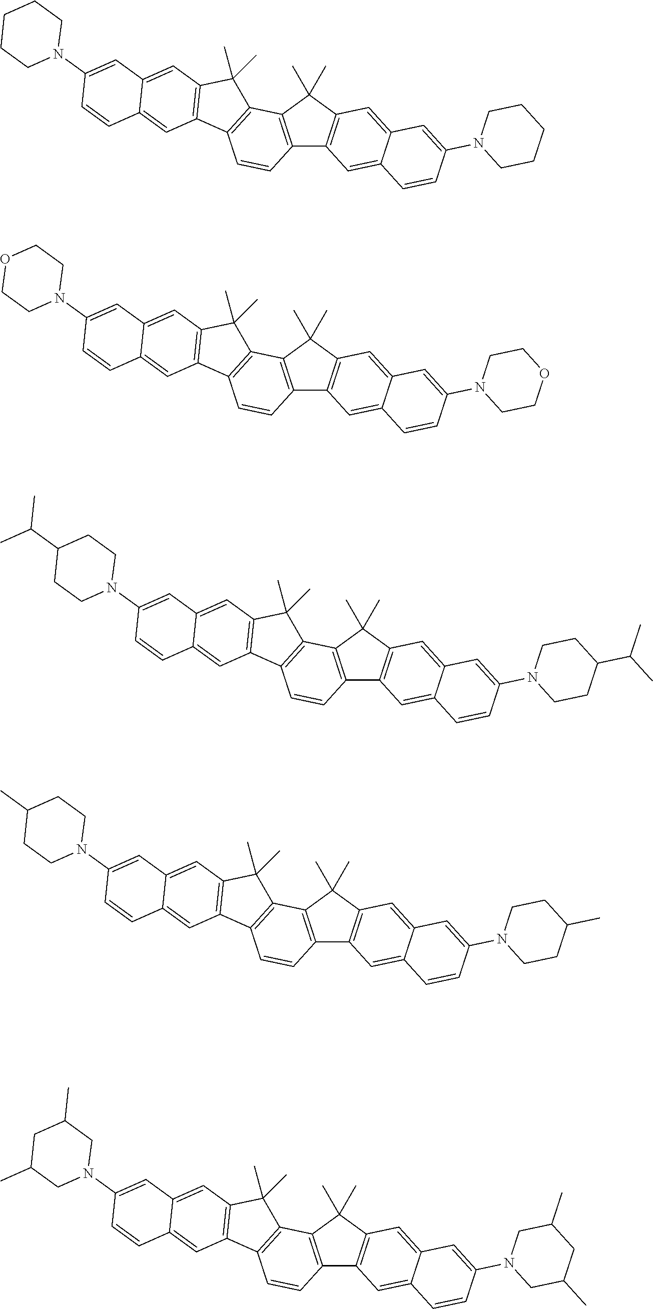

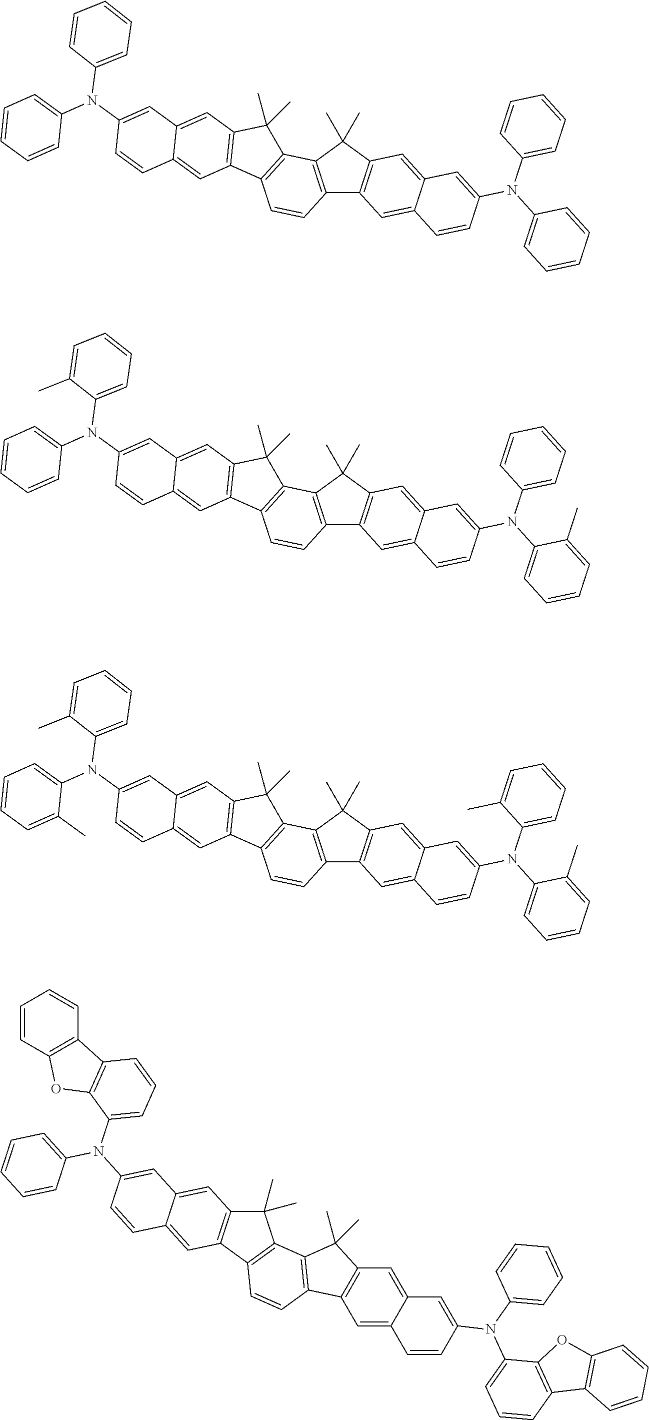 [SMILES: CC1(C)c2cc3cc(N(c4ccccc4)c4ccccc4)ccc3cc2-c2ccc3c(c21)C(C)(C)c1cc2cc(N(c4ccccc4)c4ccccc4)ccc2cc1-3 ; Cc1ccccc1N(c1ccccc1)c1ccc2cc3c(cc2c1)C(C)(C)c1c-3ccc2c1C(C)(C)c1cc3cc(N(c4ccccc4)c4ccccc4C)ccc3cc1-2 ; Cc1ccccc1N(c1ccc2cc3c(cc2c1)C(C)(C)c1c-3ccc2c1C(C)(C)c1cc3cc(N(c4ccccc4C)c4ccccc4C)ccc3cc1-2)c1ccccc1C ; CC1(C)c2cc3cc(N(c4ccccc4)c4cccc5c4oc4ccccc45)ccc3cc2-c2ccc3c(c21)C(C)(C)c1cc2cc(N(c4ccccc4)c4cccc5c4oc4ccccc45)ccc2cc1-3]

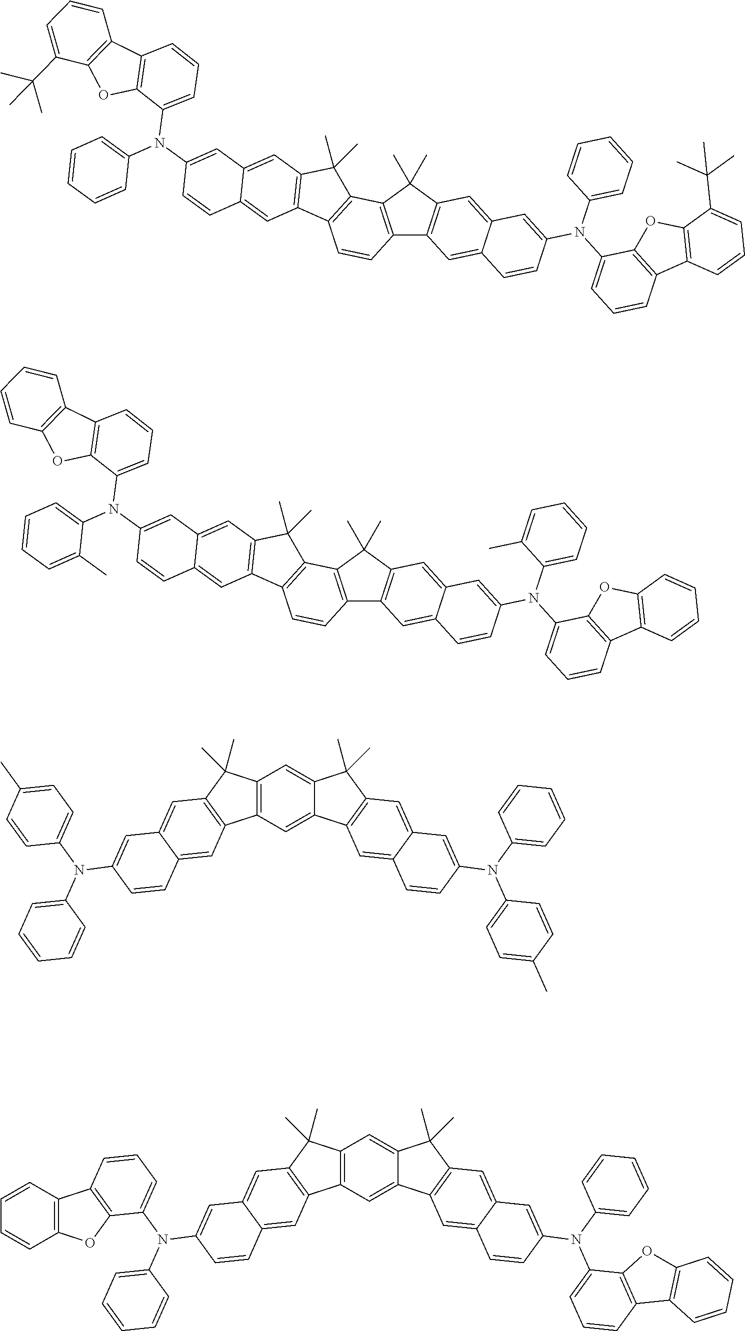 [SMILES: CC(C)(C)c1cccc2c1oc1c(N(c3ccccc3)c3ccc4cc5c(cc4c3)C(C)(C)c3c-5ccc4c3C(C)(C)c3cc5cc(N(c6ccccc6)c6cccc7c6oc6c(C(C)(C)C)cccc67)ccc5cc3-4)cccc12 ; Cc1ccccc1N(c1ccc2cc3c(cc2c1)C(C)(C)c1c-3ccc2c1C(C)(C)c1cc3cc(N(c4ccccc4C)c4cccc5c4oc4ccccc45)ccc3cc1-2)c1cccc2c1oc1ccccc12 ; Cc1ccc(N(c2ccccc2)c2ccc3cc4c(cc3c2)C(C)(C)c2cc3c(cc2-4)-c2cc4ccc(N(c5ccccc5)c5ccc(C)cc5)cc4cc2C3(C)C)cc1 ; CC1(C)c2cc3c(cc2-c2cc4ccc(N(c5ccccc5)c5cccc6c5oc5ccccc56)cc4cc21)-c1cc2ccc(N(c4ccccc4)c4cccc5c4oc4ccccc45)cc2cc1C3(C)C]

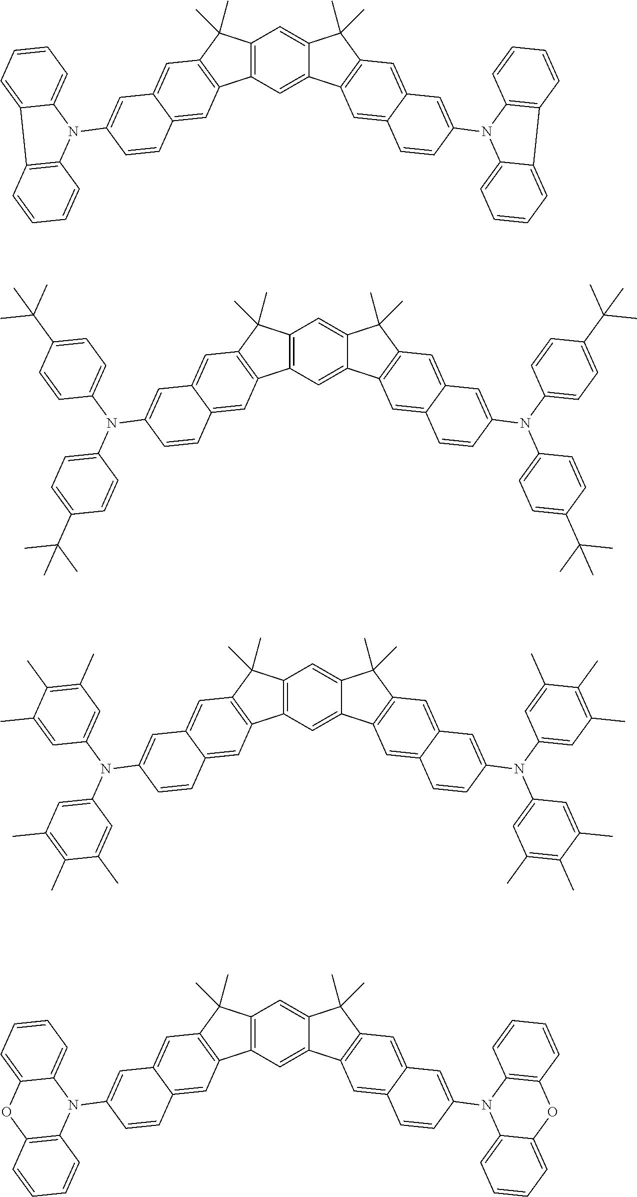

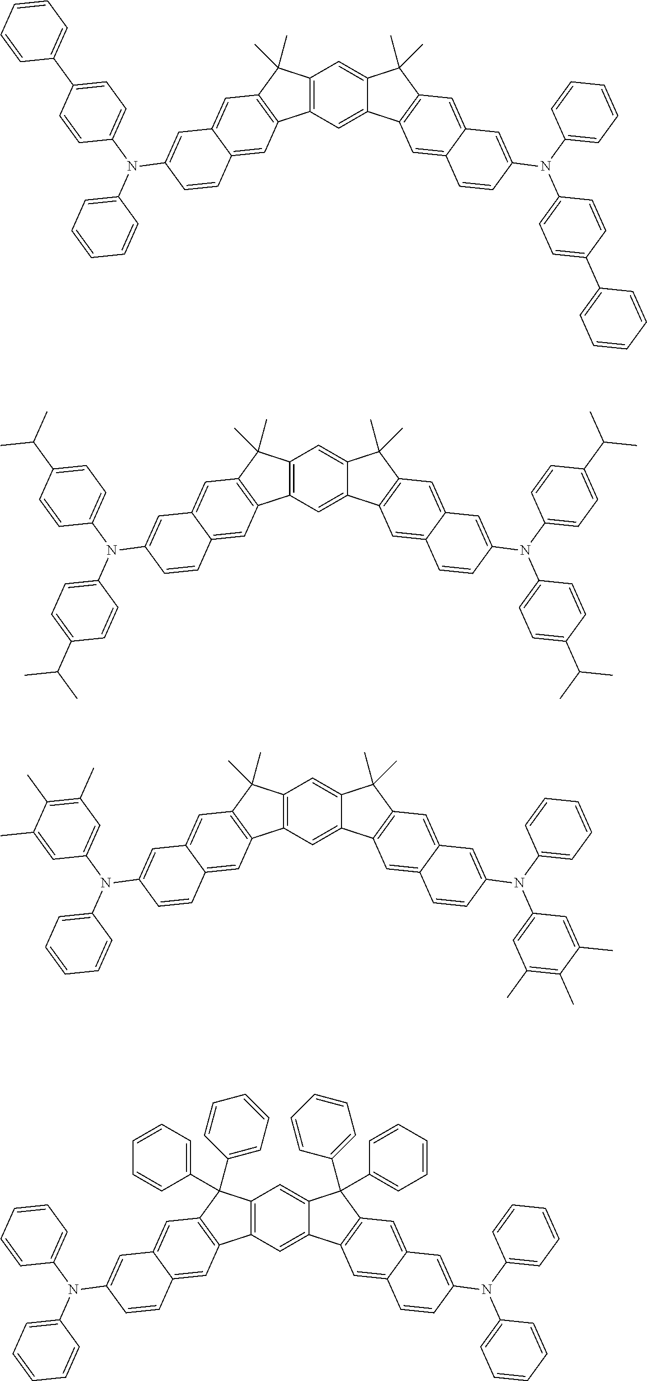

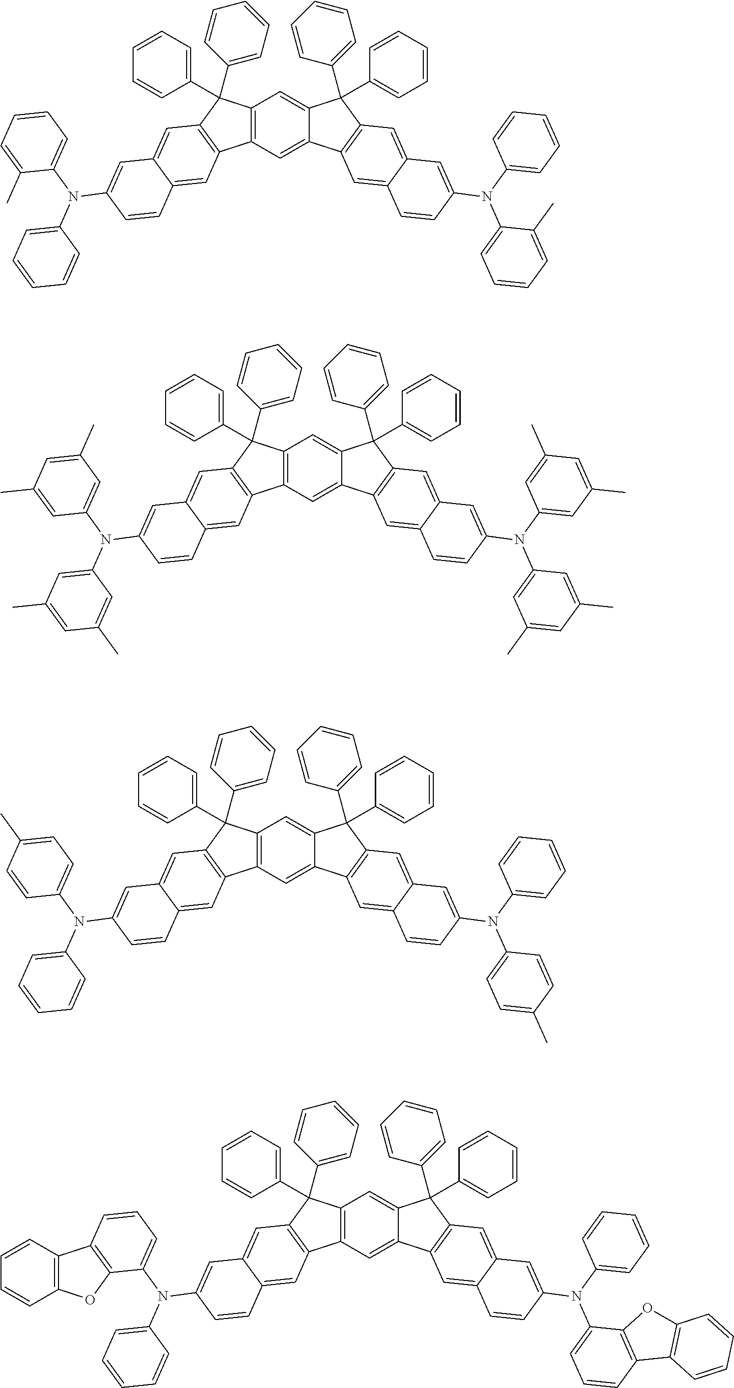

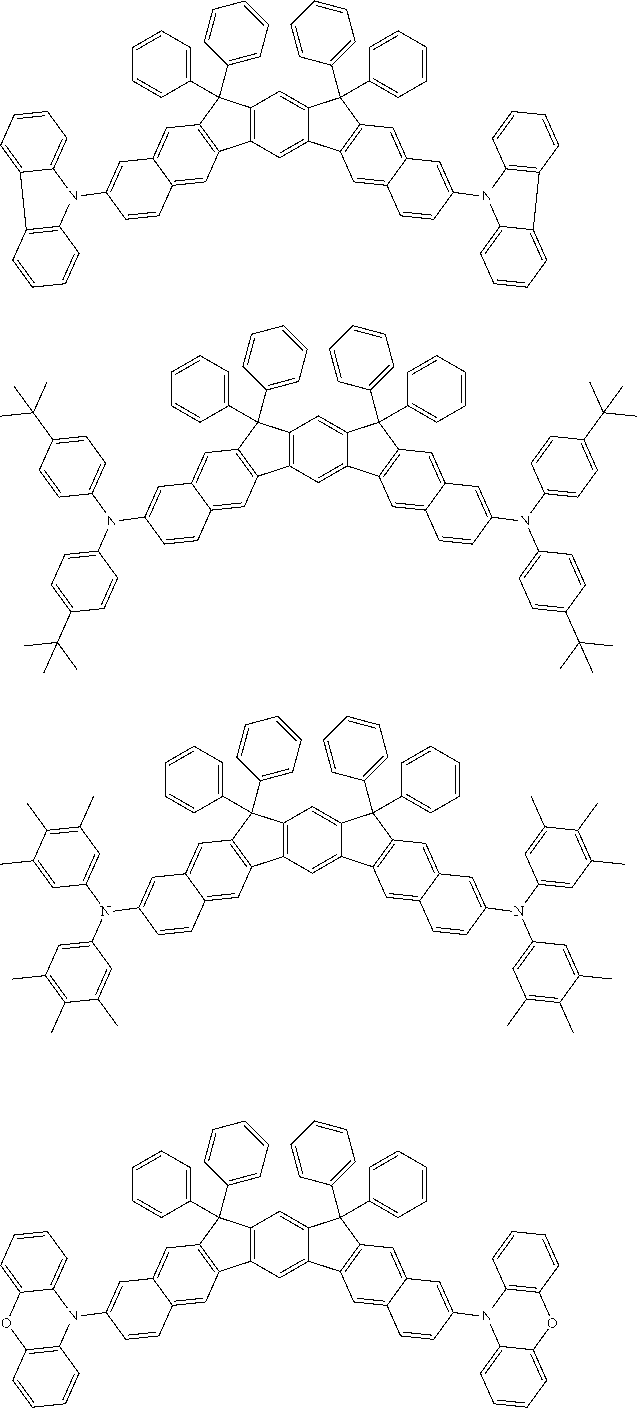

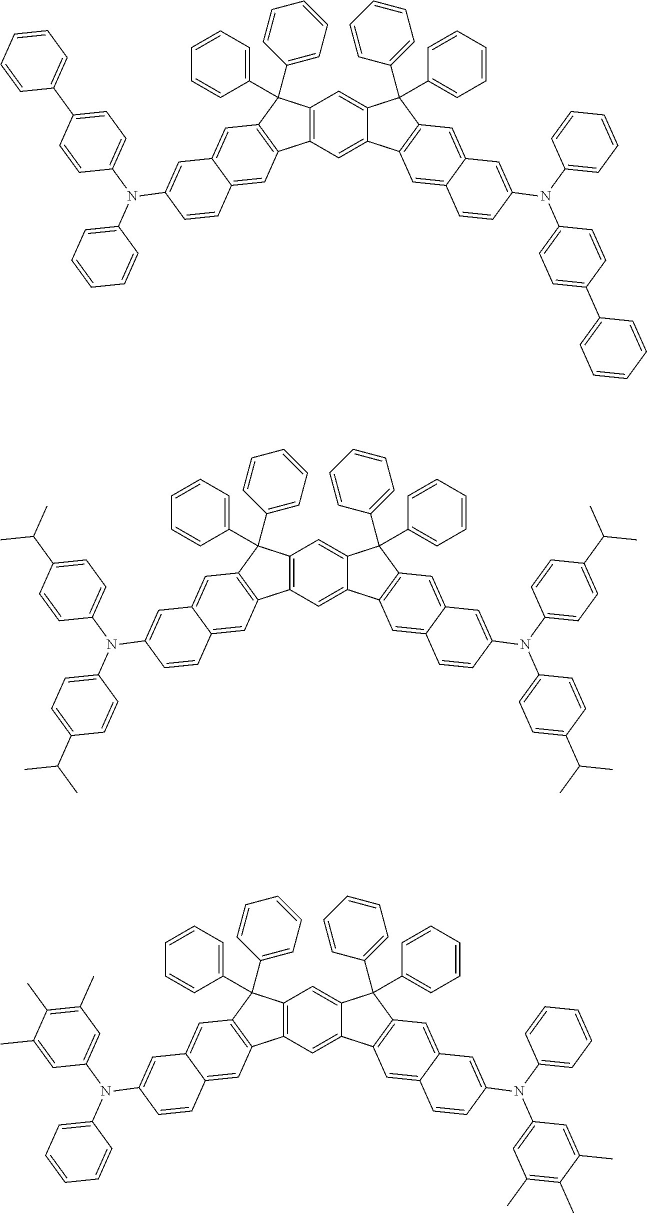

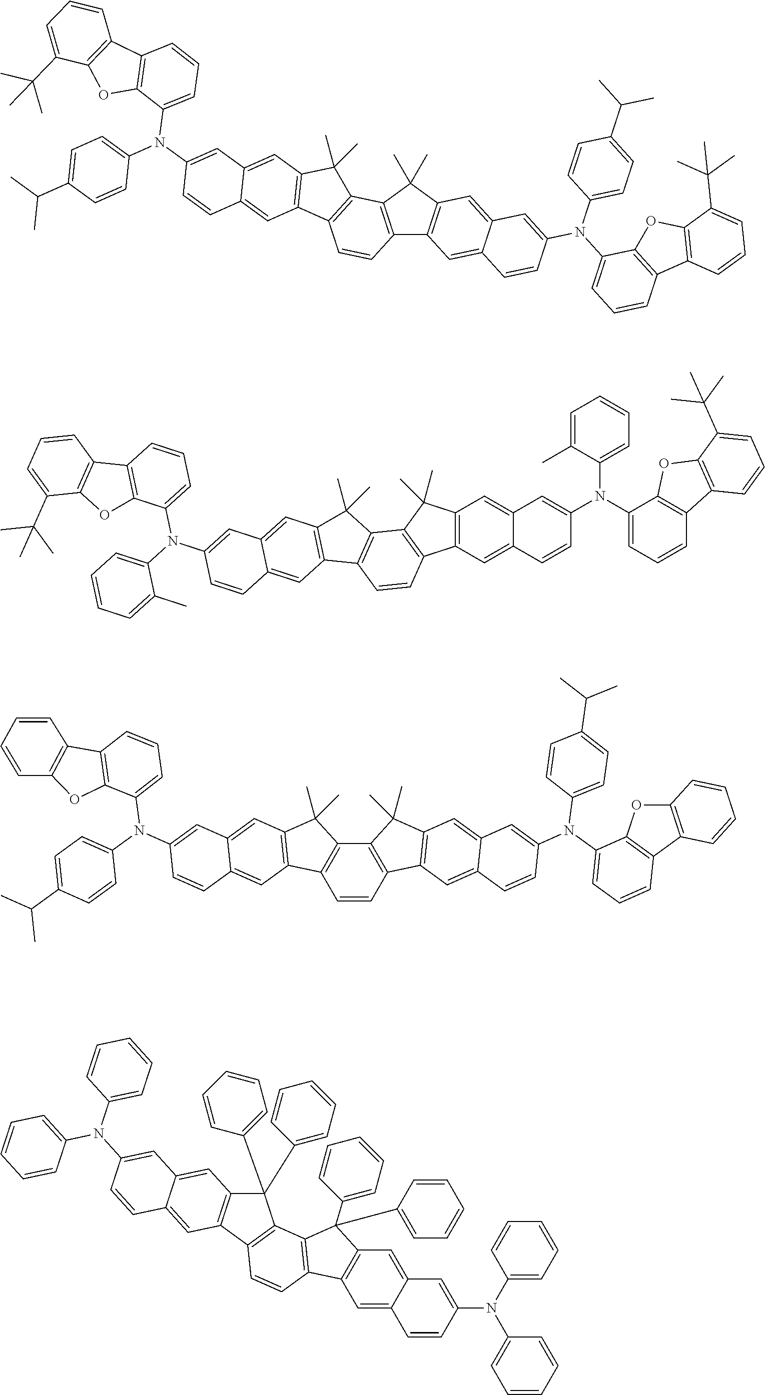

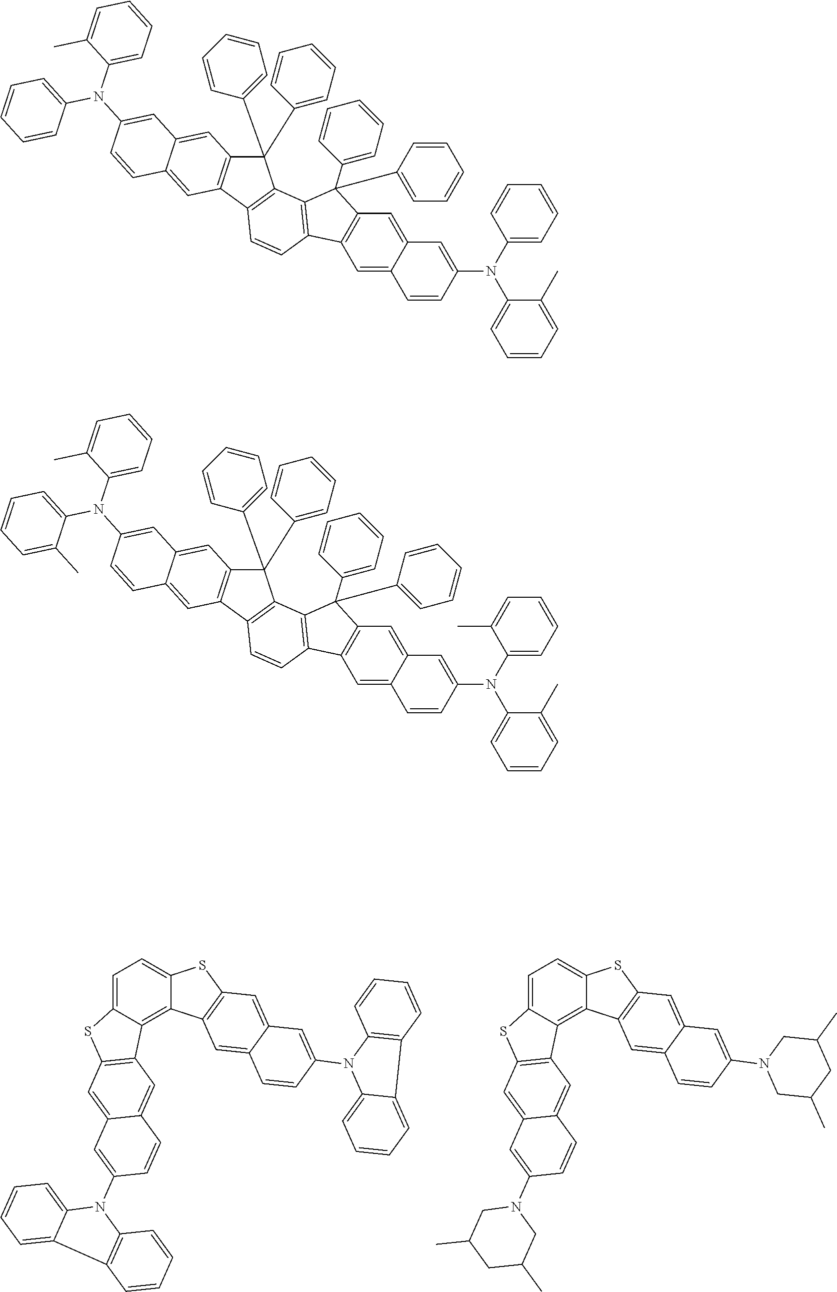

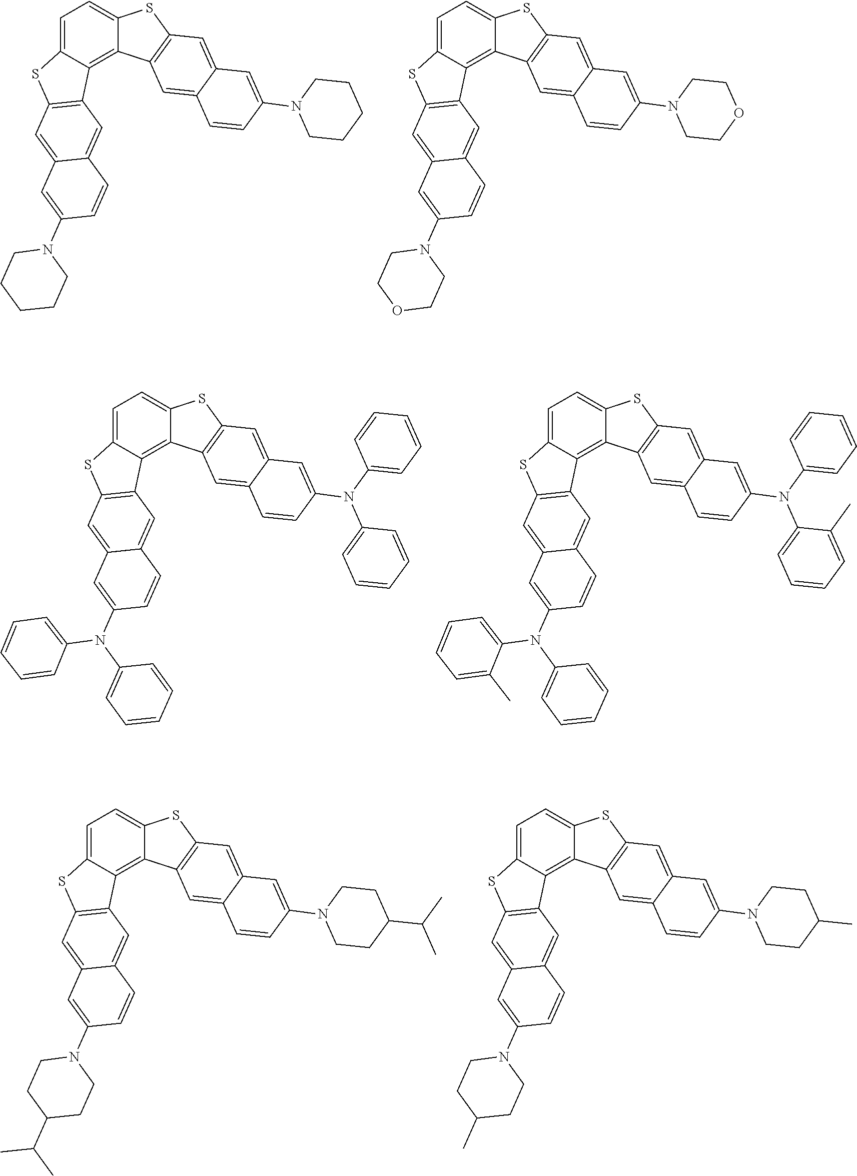 [SMILES: c1cc2cc3c(cc2cc1N1CCCCC1)sc1ccc2sc4cc5cc(N6CCCCC6)ccc5cc4c2c13 ; c1cc2cc3c(cc2cc1N1CCOCC1)sc1ccc2sc4cc5cc(N6CCOCC6)ccc5cc4c2c13 ; c1ccc(N(c2ccccc2)c2ccc3cc4c(cc3c2)sc2ccc3sc5cc6cc(N(c7ccccc7)c7ccccc7)ccc6cc5c3c24)cc1 ; Cc1ccccc1N(c1ccccc1)c1ccc2cc3c(cc2c1)sc1ccc2sc4cc5cc(N(c6ccccc6)c6ccccc6C)ccc5cc4c2c13 ; CC(C)C1CCN(c2ccc3cc4c(cc3c2)sc2ccc3sc5cc6cc(N7CCC(C(C)C)CC7)ccc6cc5c3c24)CC1 ; CC1CCN(c2ccc3cc4c(cc3c2)sc2ccc3sc5cc6cc(N7CCC(C)CC7)ccc6cc5c3c24)CC1]

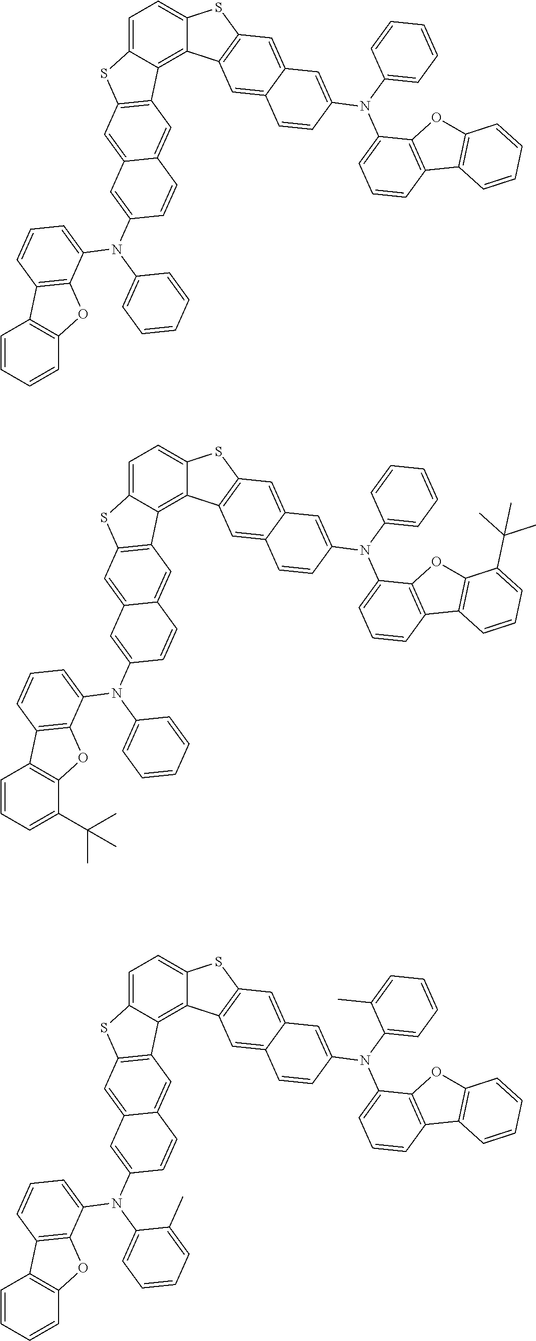 [SMILES: c1ccc(N(c2ccc3cc4c(cc3c2)sc2ccc3sc5cc6cc(N(c7ccccc7)c7cccc8c7oc7ccccc78)ccc6cc5c3c24)c2cccc3c2oc2ccccc23)cc1 ; CC(C)(C)c1cccc2c1oc1c(N(c3ccccc3)c3ccc4cc5c(cc4c3)sc3ccc4sc6cc7cc(N(c8ccccc8)c8cccc9c8oc8c(C(C)(C)C)cccc89)ccc7cc6c4c35)cccc12 ; Cc1ccccc1N(c1ccc2cc3c(cc2c1)sc1ccc2sc4cc5cc(N(c6ccccc6C)c6cccc7c6oc6ccccc67)ccc5cc4c2c13)c1cccc2c1oc1ccccc12]

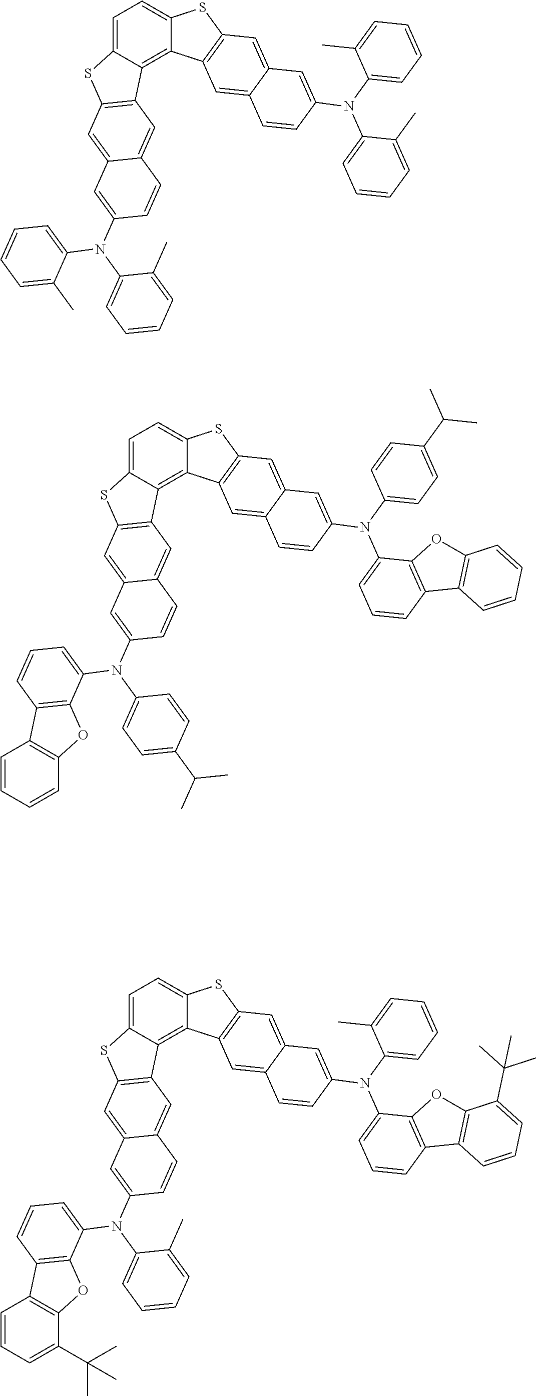 [SMILES: Cc1ccccc1N(c1ccc2cc3c(cc2c1)sc1ccc2sc4cc5cc(N(c6ccccc6C)c6ccccc6C)ccc5cc4c2c13)c1ccccc1C ; CC(C)c1ccc(N(c2ccc3cc4c(cc3c2)sc2ccc3sc5cc6cc(N(c7ccc(C(C)C)cc7)c7cccc8c7oc7ccccc78)ccc6cc5c3c24)c2cccc3c2oc2ccccc23)cc1 ; Cc1ccccc1N(c1ccc2cc3c(cc2c1)sc1ccc2sc4cc5cc(N(c6ccccc6C)c6cccc7c6oc6c(C(C)(C)C)cccc67)ccc5cc4c2c13)c1cccc2c1oc1c(C(C)(C)C)cccc12]

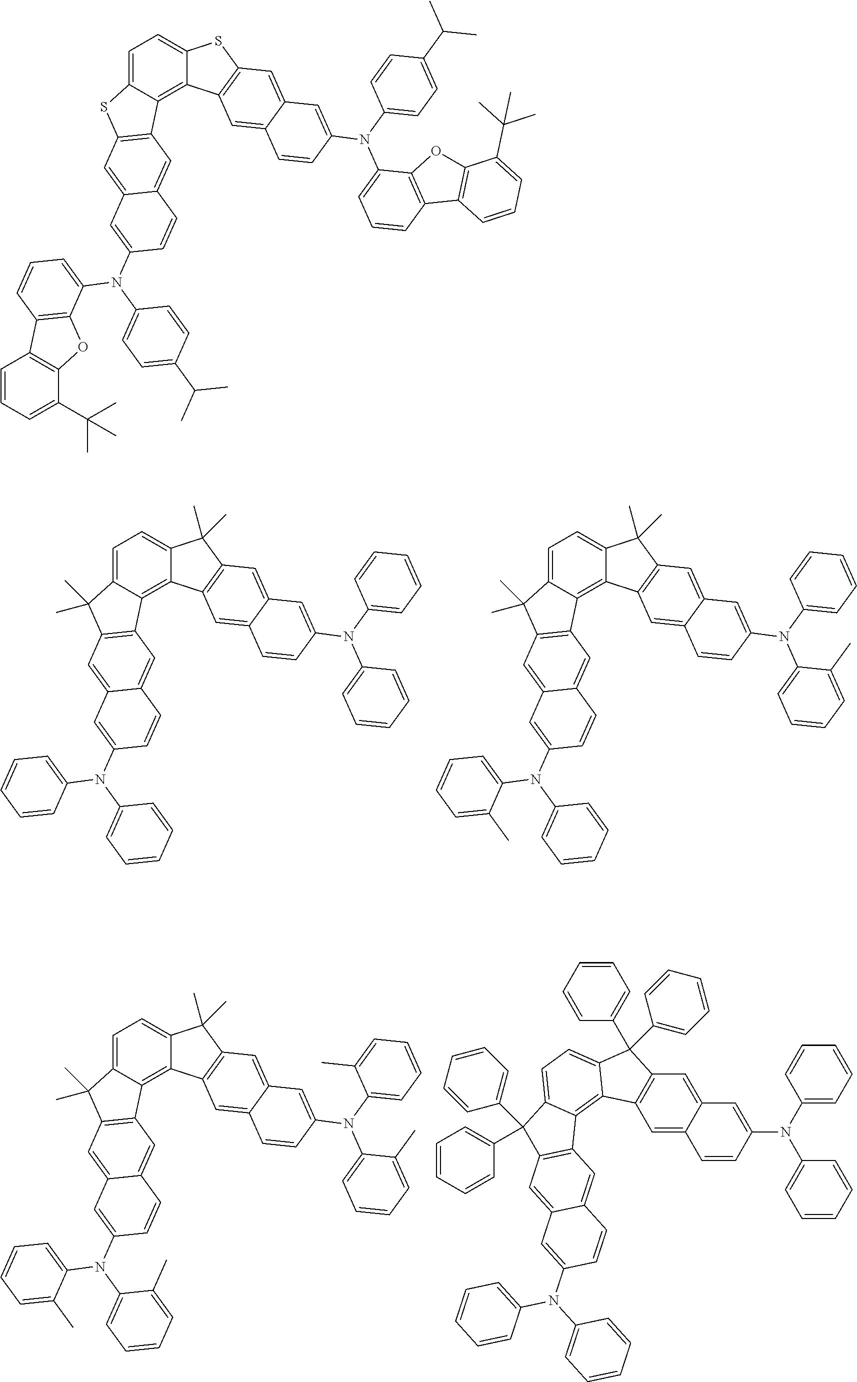

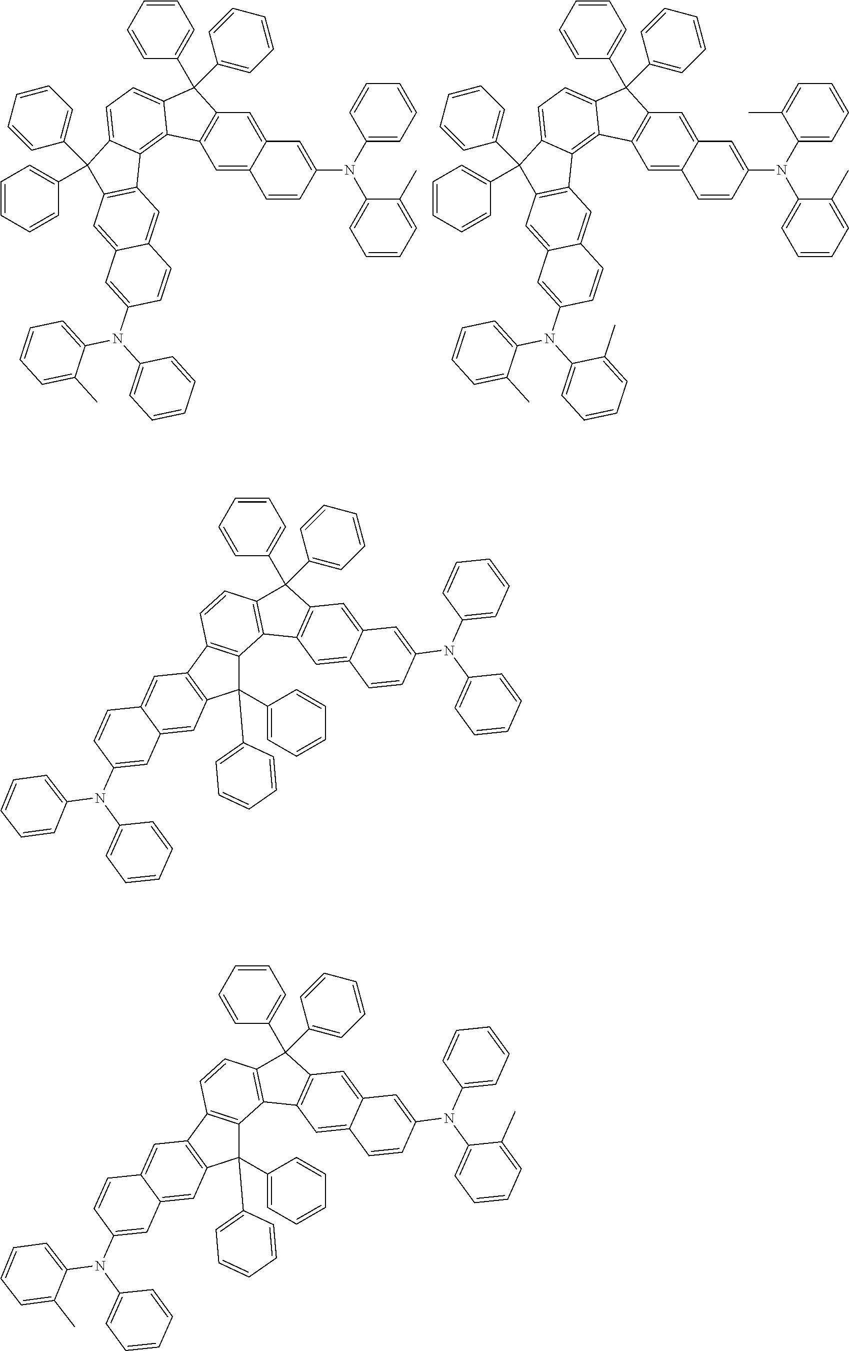

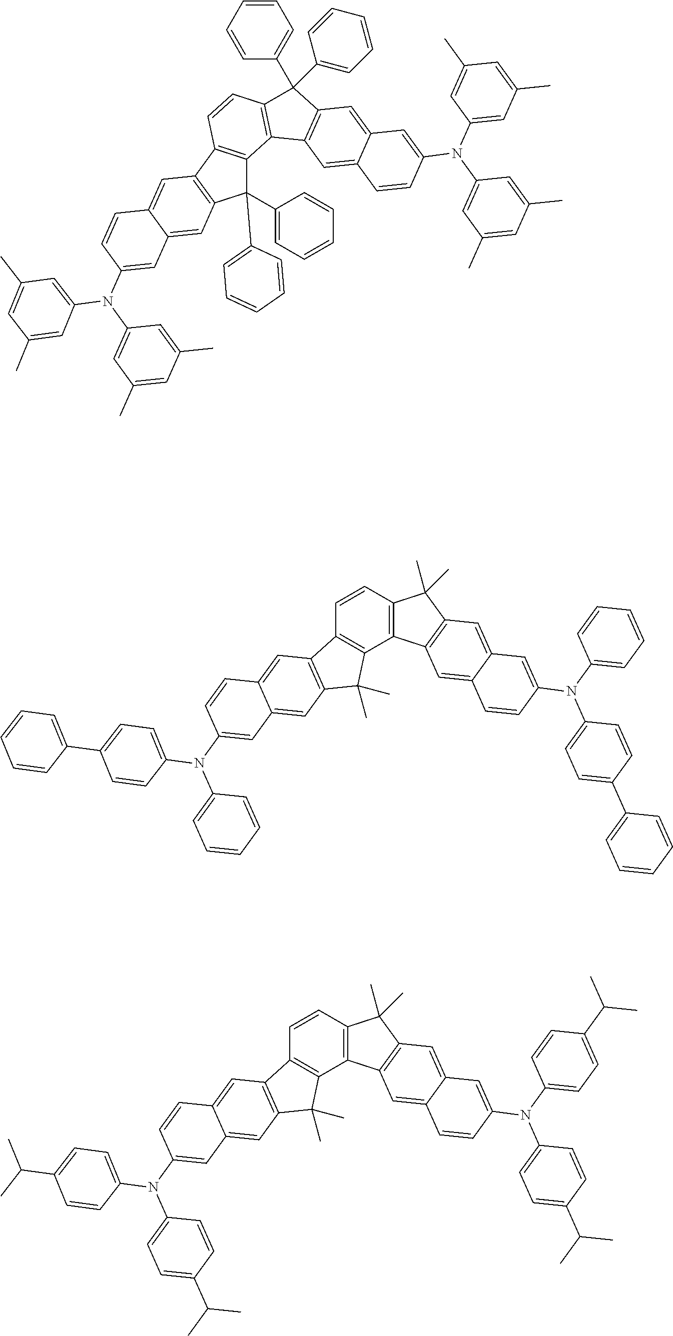

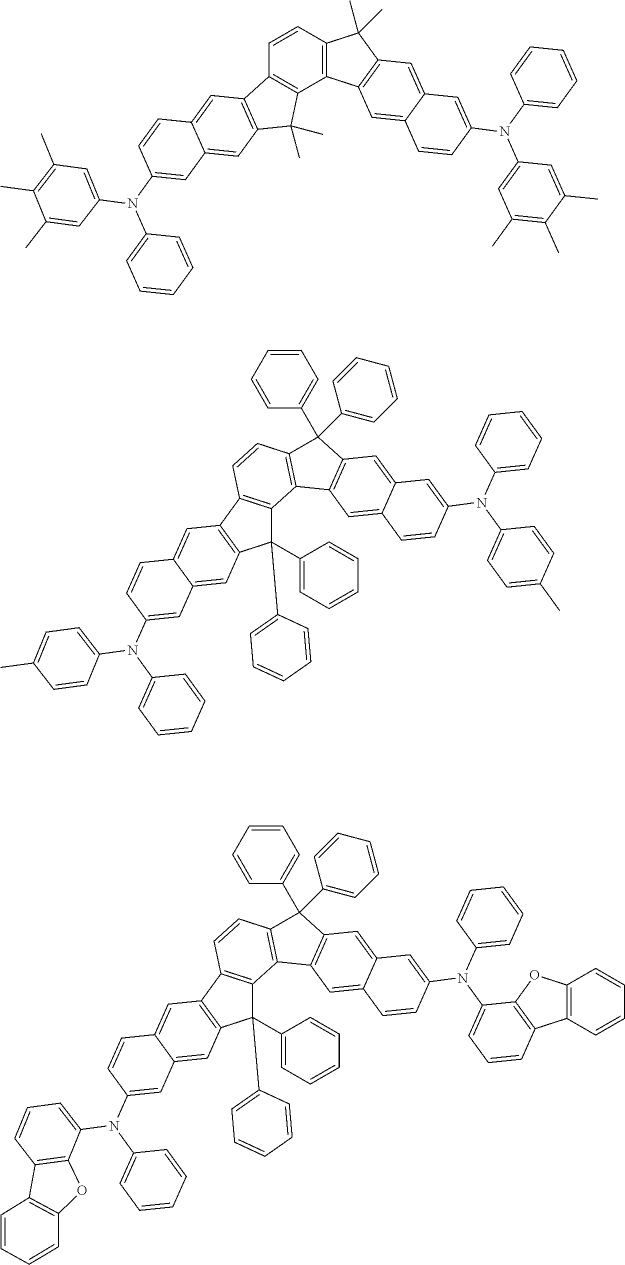

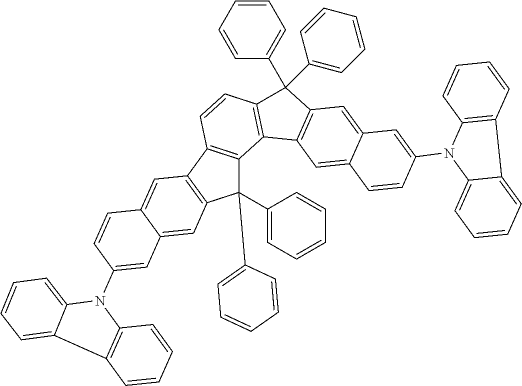 [SMILES: c1ccc(C2(c3ccccc3)c3cc4cc(-n5c6ccccc6c6ccccc65)ccc4cc3-c3c2ccc2c3C(c3ccccc3)(c3ccccc3)c3cc4cc(-n5c6ccccc6c6ccccc65)ccc4cc3-2)cc1]

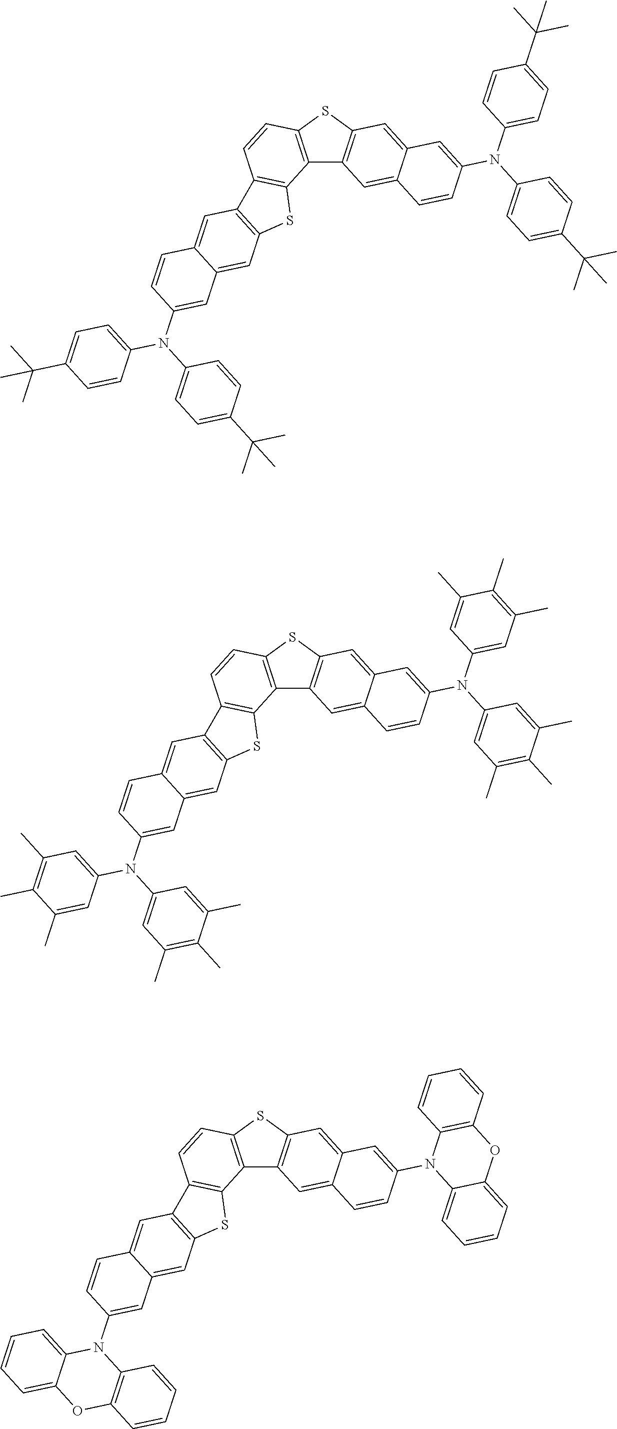

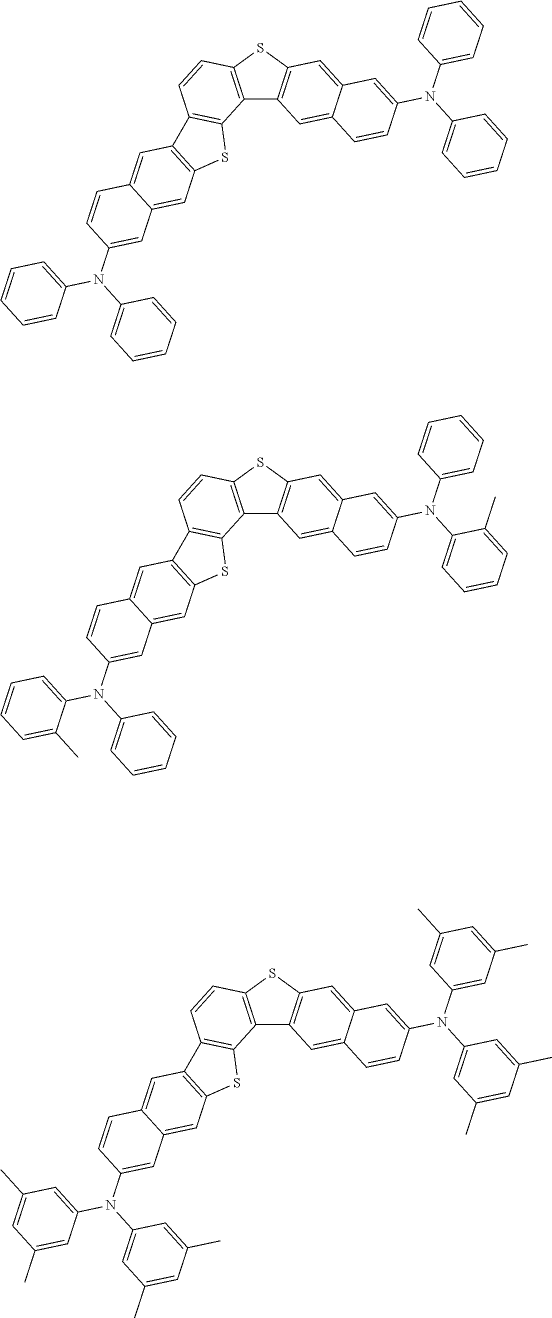

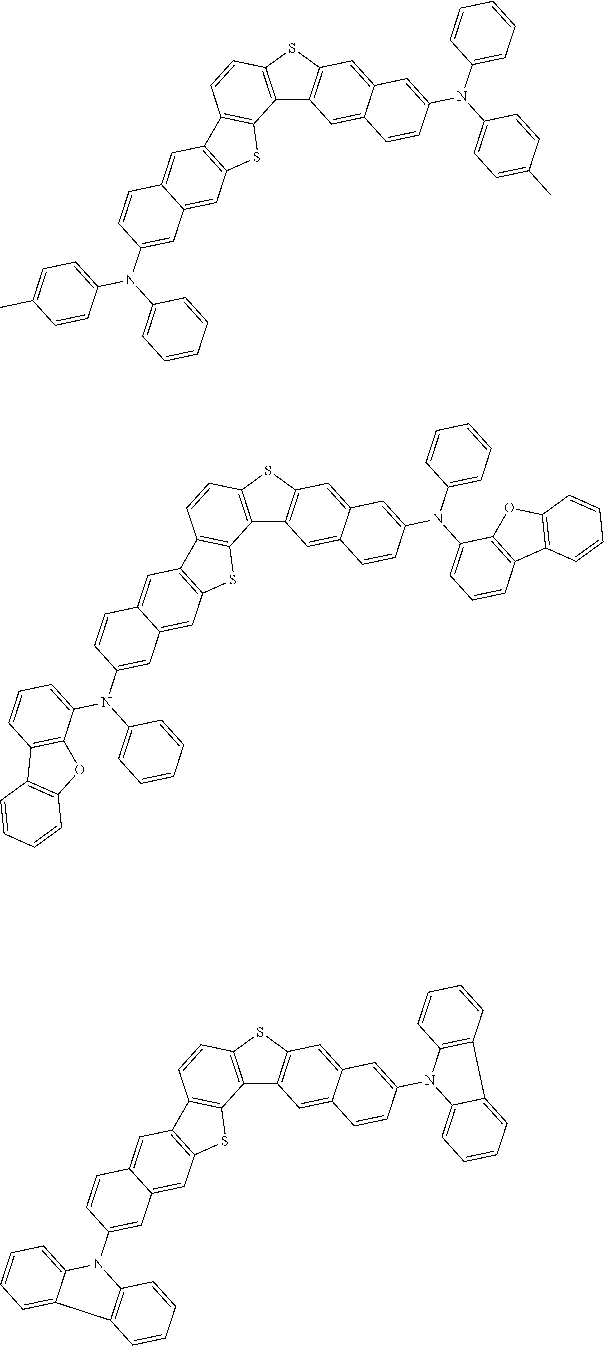

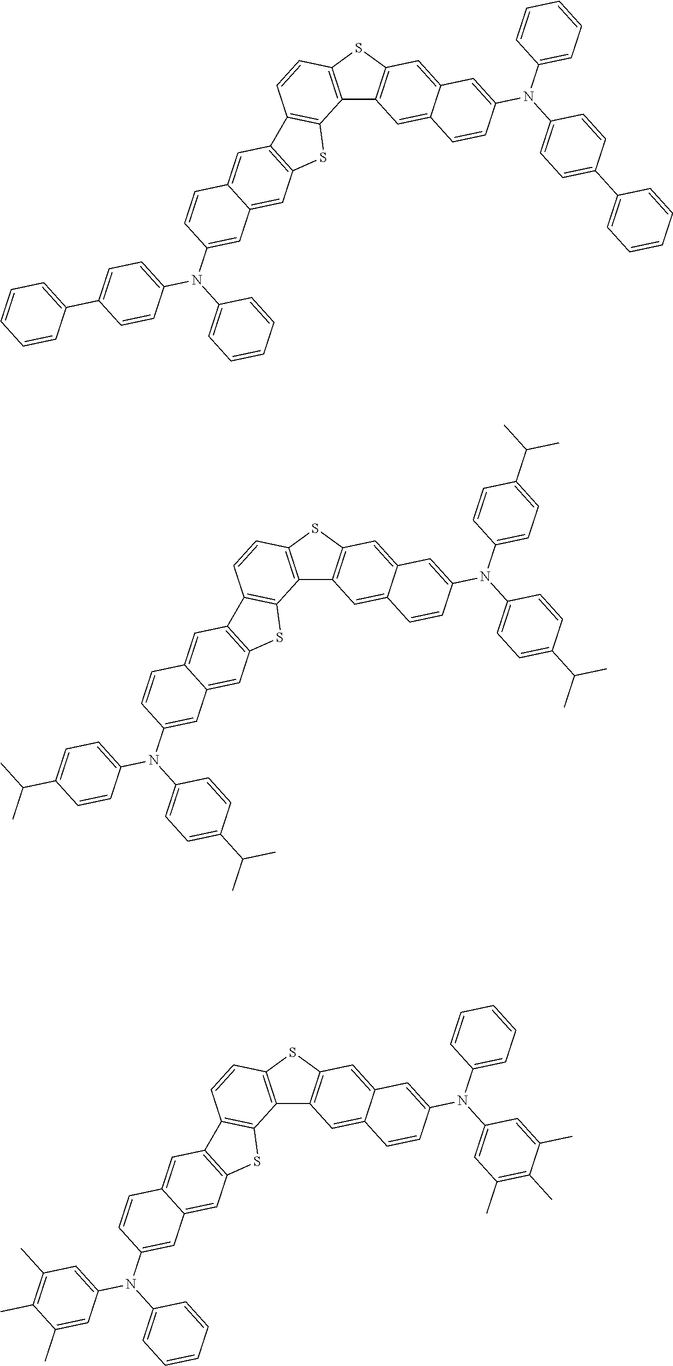

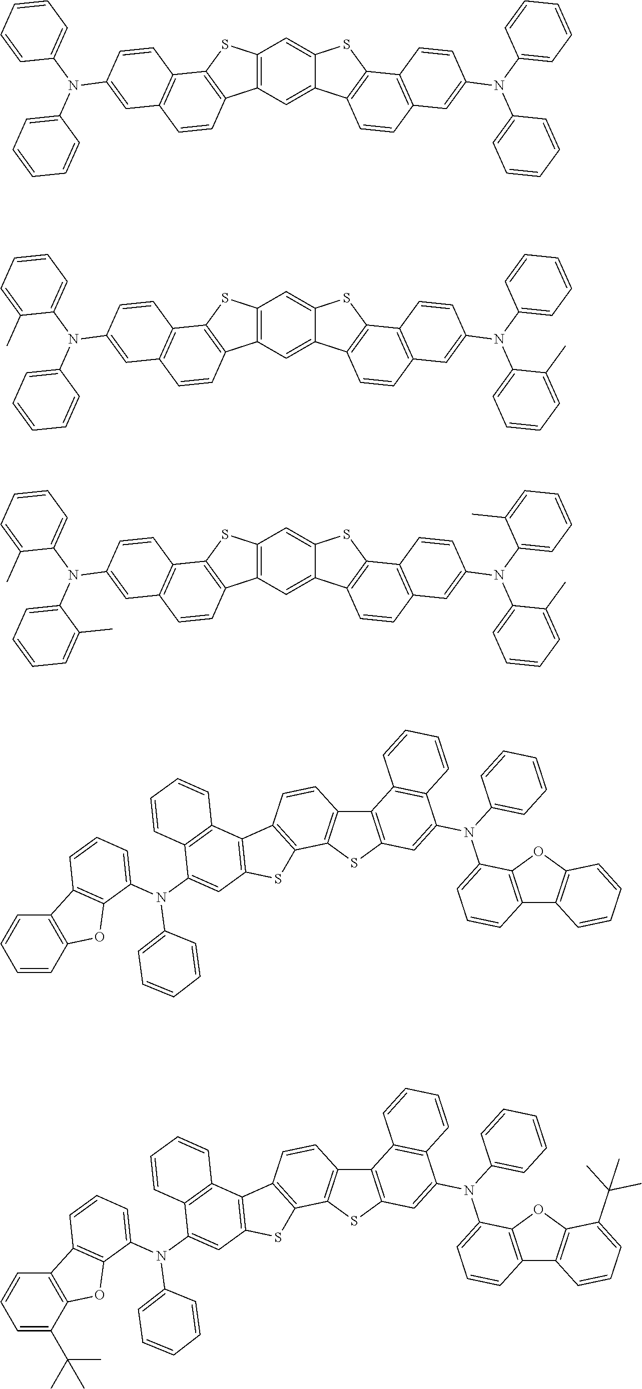

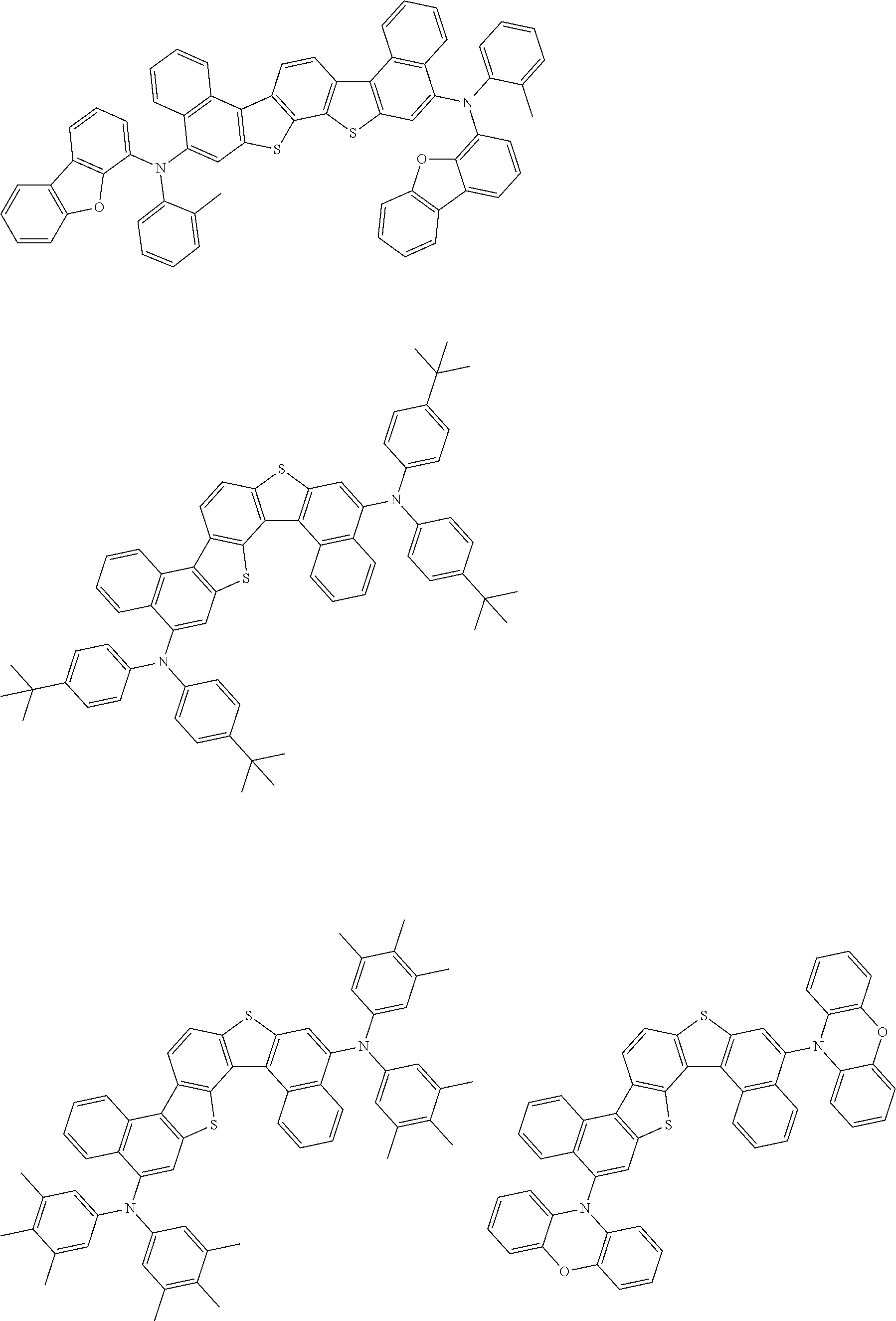

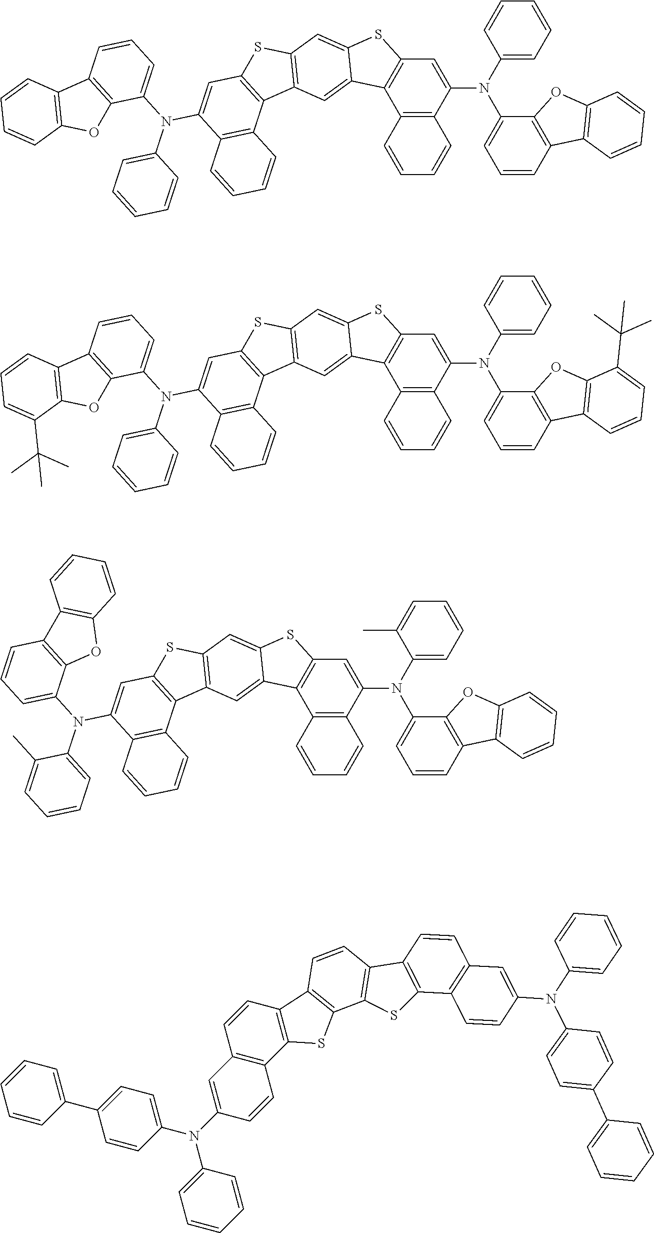 [SMILES: c1ccc(N(c2cc3sc4cc5sc6cc(N(c7ccccc7)c7cccc8c7oc7ccccc78)c7ccccc7c6c5cc4c3c3ccccc23)c2cccc3c2oc2ccccc23)cc1 ; CC(C)(C)c1cccc2c1oc1c(N(c3ccccc3)c3cc4sc5cc6sc7cc(N(c8ccccc8)c8cccc9c8oc8c(C(C)(C)C)cccc89)c8ccccc8c7c6cc5c4c4ccccc34)cccc12 ; Cc1ccccc1N(c1cc2sc3cc4sc5cc(N(c6ccccc6C)c6cccc7c6oc6ccccc67)c6ccccc6c5c4cc3c2c2ccccc12)c1cccc2c1oc1ccccc12 ; c1ccc(-c2ccc(N(c3ccccc3)c3ccc4c(ccc5c6ccc7c8ccc9cc(N(c%10ccccc%10)c%10ccc(-c%11ccccc%11)cc%10)ccc9c8sc7c6sc45)c3)cc2)cc1]

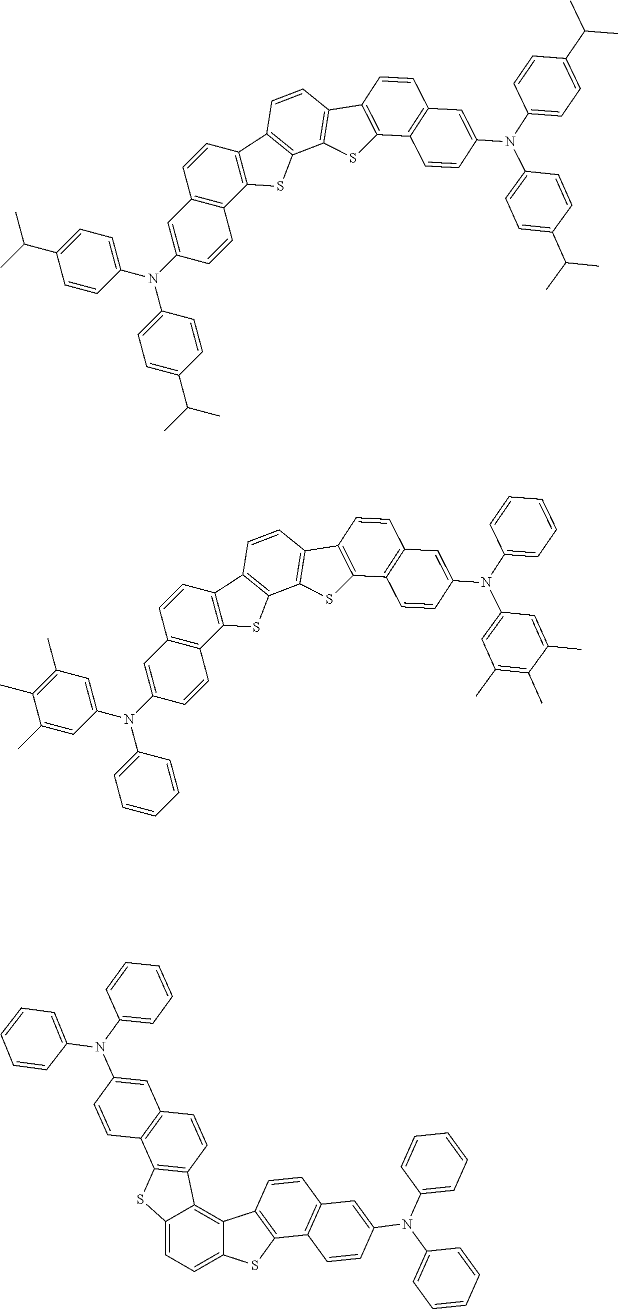

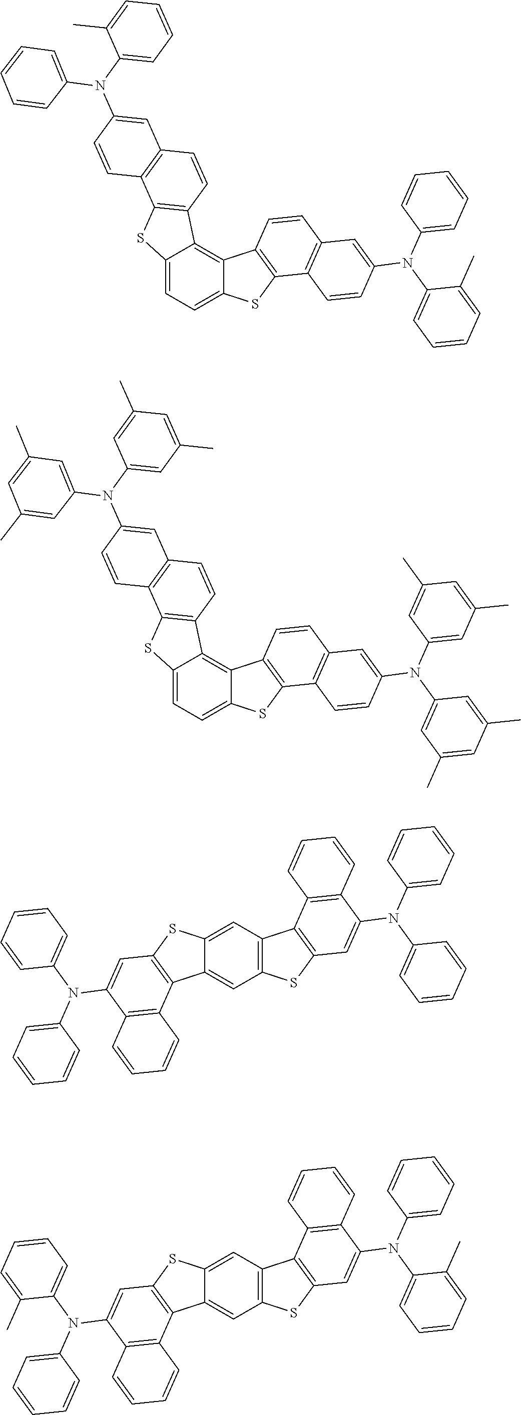

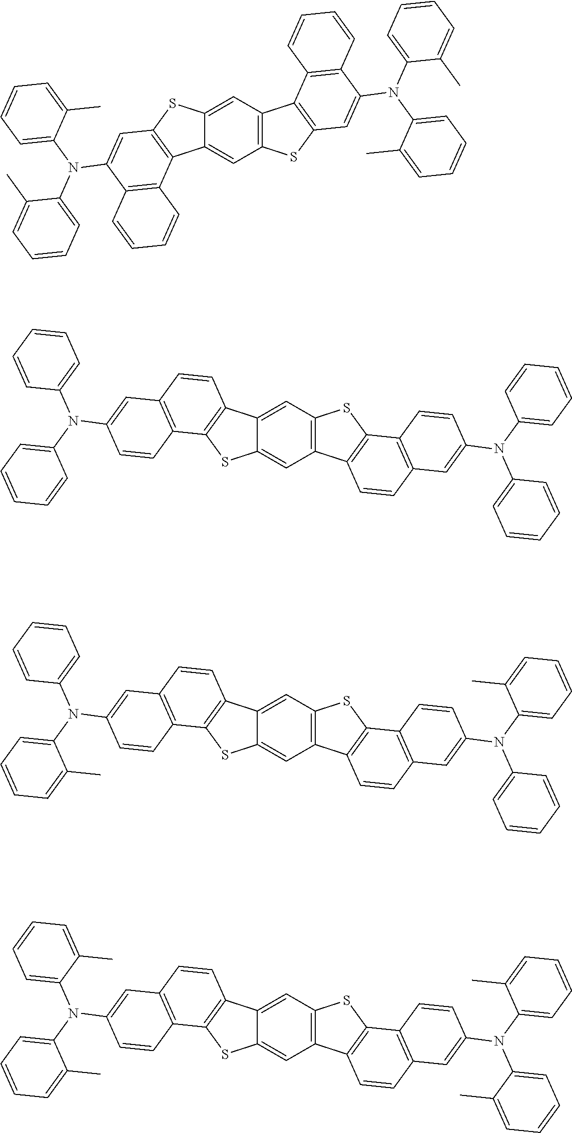 [SMILES: Cc1ccccc1N(c1ccccc1C)c1cc2sc3cc4c(cc3c2c2ccccc12)sc1cc(N(c2ccccc2C)c2ccccc2C)c2ccccc2c14 ; c1ccc(N(c2ccccc2)c2ccc3c(ccc4c5cc6sc7c8ccc(N(c9ccccc9)c9ccccc9)cc8ccc7c6cc5sc34)c2)cc1 ; Cc1ccccc1N(c1ccccc1)c1ccc2c(ccc3c4cc5sc6c7ccc(N(c8ccccc8)c8ccccc8C)cc7ccc6c5cc4sc23)c1 ; Cc1ccccc1N(c1ccc2c(ccc3c4cc5sc6c7ccc(N(c8ccccc8C)c8ccccc8C)cc7ccc6c5cc4sc23)c1)c1ccccc1C]

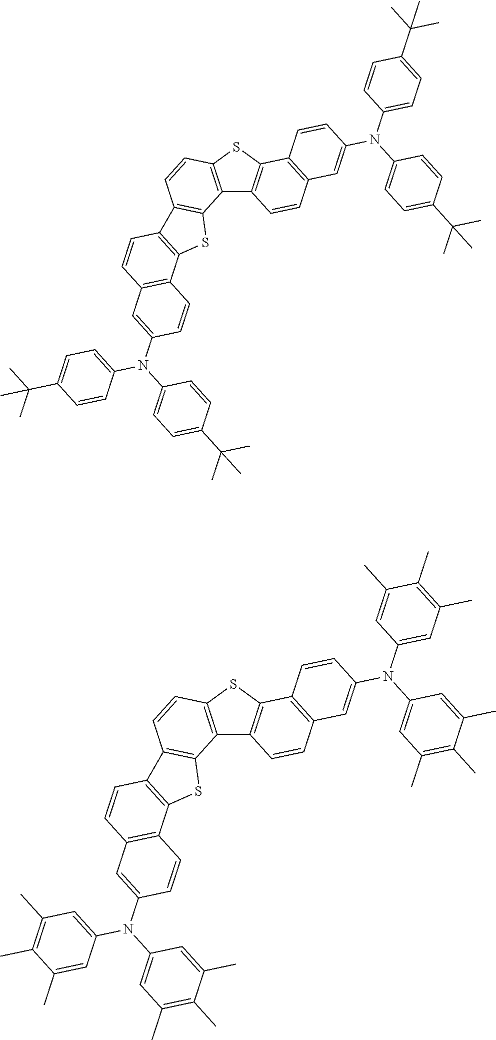

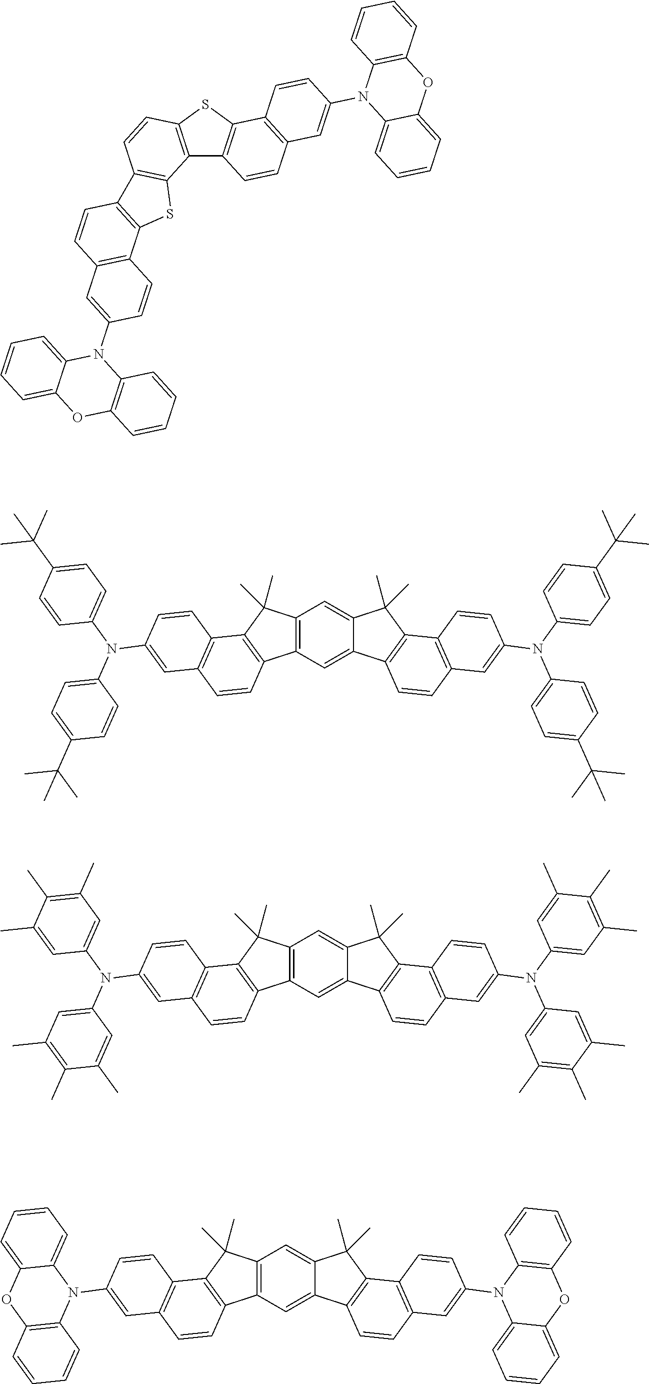

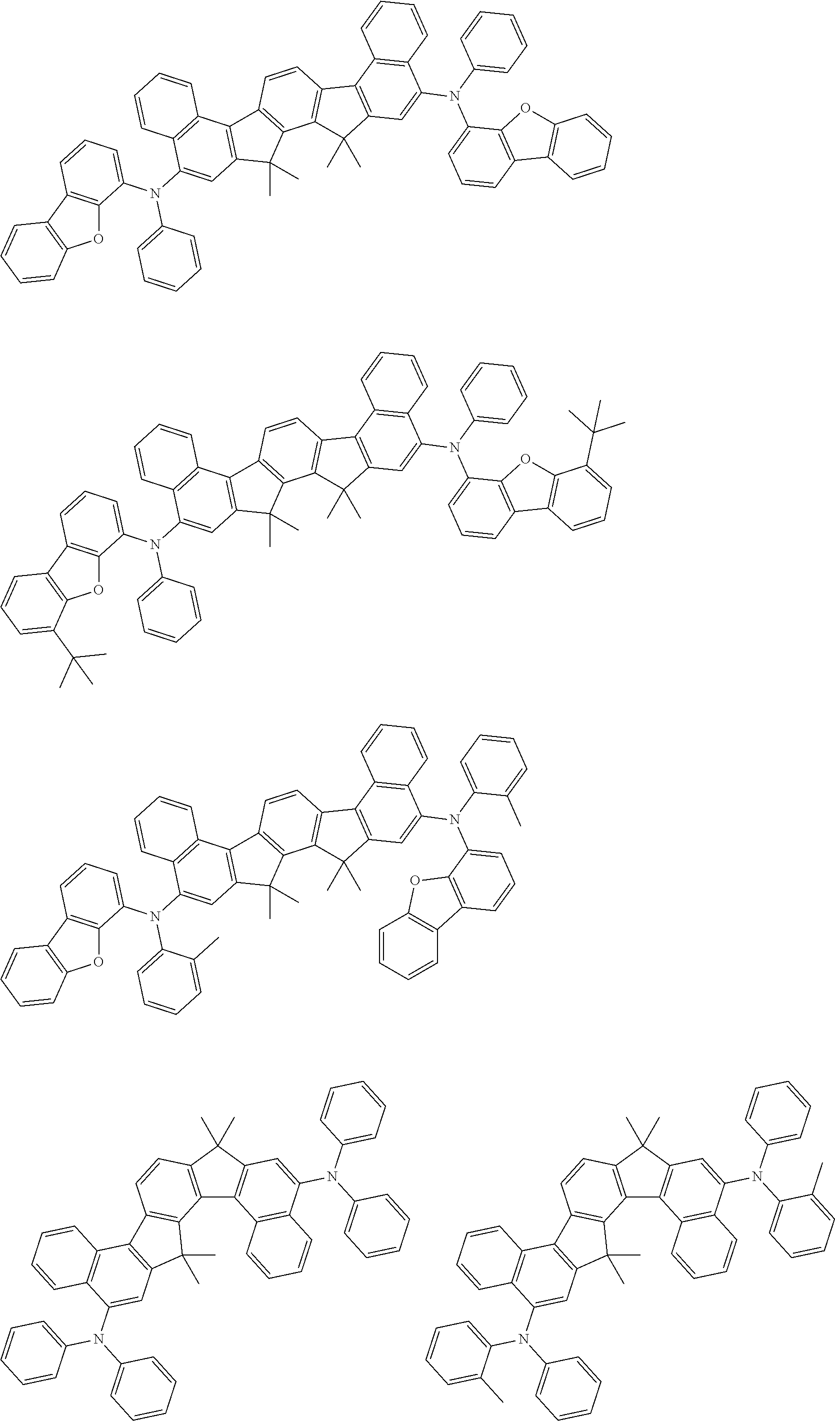

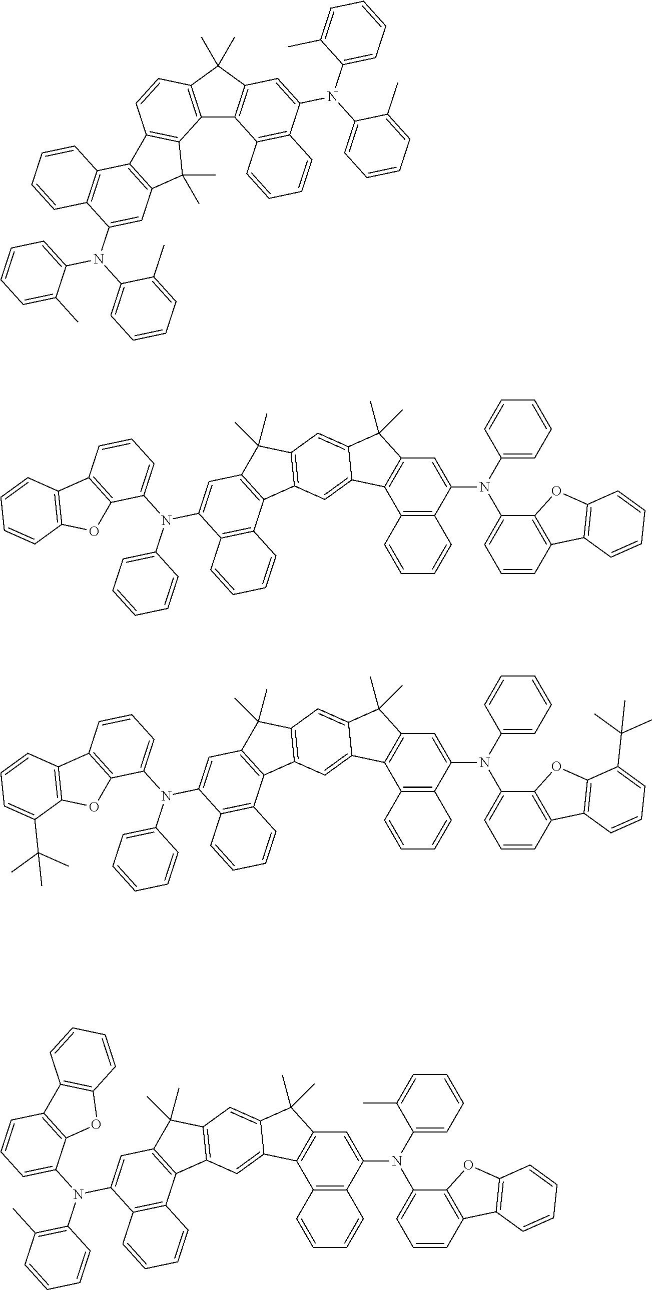 [SMILES: Cc1ccccc1N(c1ccccc1C)c1cc2c(c3ccccc13)-c1ccc3c(c1C2(C)C)-c1c(cc(N(c2ccccc2C)c2ccccc2C)c2ccccc12)C3(C)C ; CC1(C)c2cc3c(cc2-c2c1cc(N(c1ccccc1)c1cccc4c1oc1ccccc14)c1ccccc21)-c1c(cc(N(c2ccccc2)c2cccc4c2oc2ccccc24)c2ccccc12)C3(C)C ; CC(C)(C)c1cccc2c1oc1c(N(c3ccccc3)c3cc4c(c5ccccc35)-c3cc5c(cc3C4(C)C)C(C)(C)c3cc(N(c4ccccc4)c4cccc6c4oc4c(C(C)(C)C)cccc46)c4ccccc4c3-5)cccc12 ; Cc1ccccc1N(c1cc2c(c3ccccc13)-c1cc3c(cc1C2(C)C)C(C)(C)c1cc(N(c2ccccc2C)c2cccc4c2oc2ccccc24)c2ccccc2c1-3)c1cccc2c1oc1ccccc12]

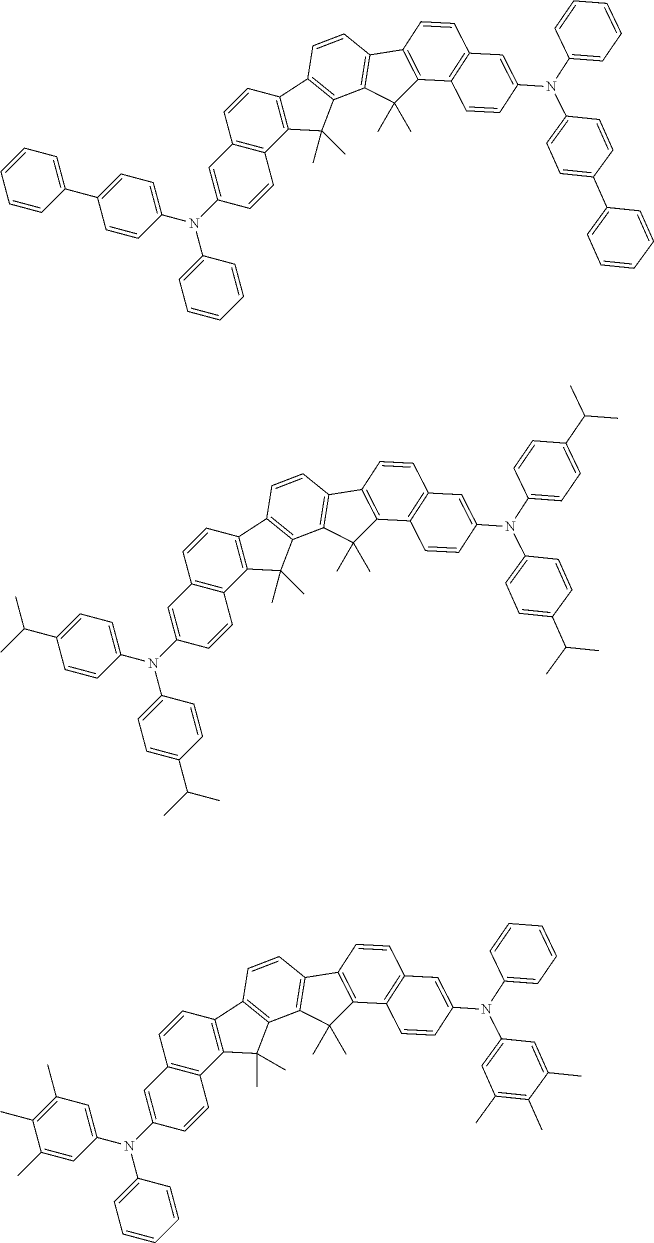

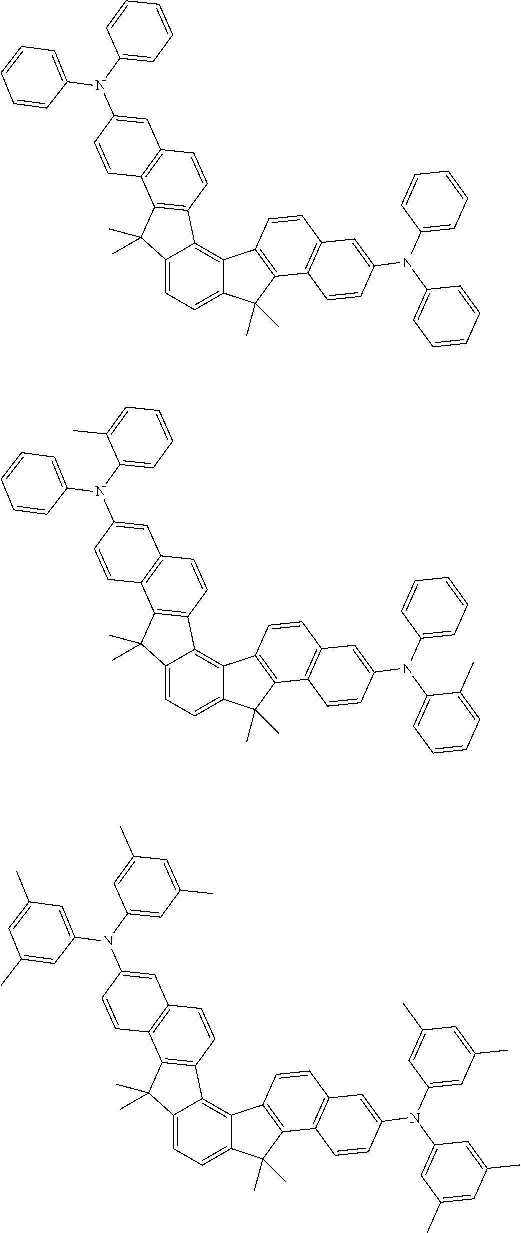

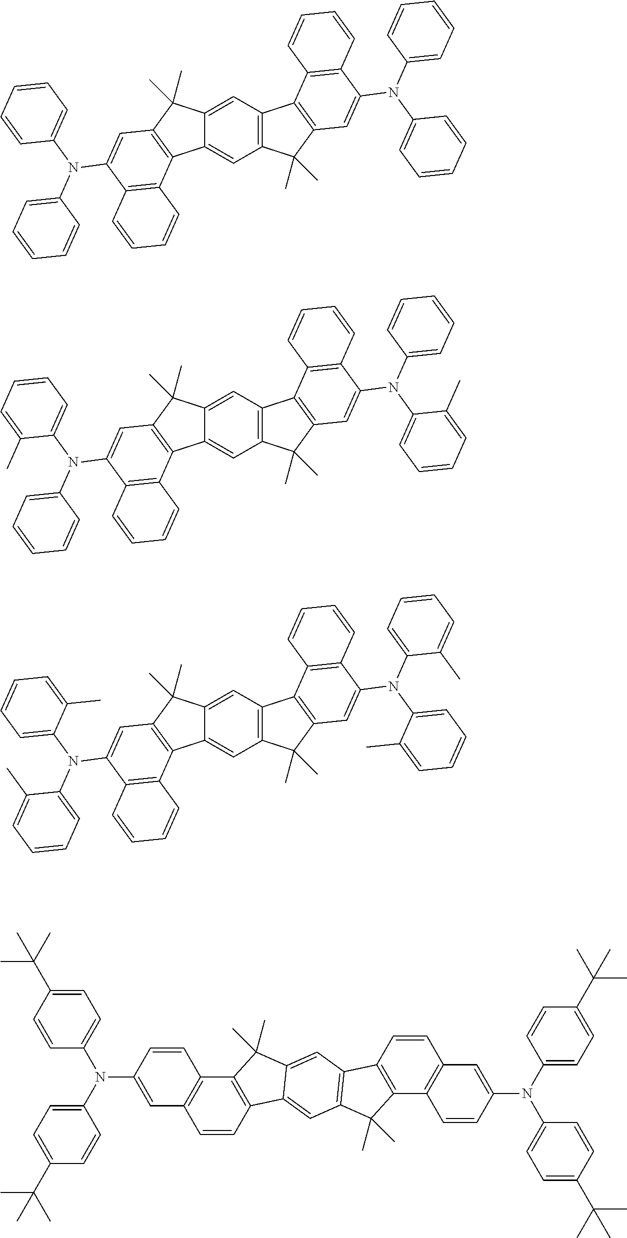 [SMILES: CC1(C)c2cc3c(cc2-c2c1cc(N(c1ccccc1)c1ccccc1)c1ccccc21)C(C)(C)c1cc(N(c2ccccc2)c2ccccc2)c2ccccc2c1-3 ; Cc1ccccc1N(c1ccccc1)c1cc2c(c3ccccc13)-c1cc3c(cc1C2(C)C)-c1c(cc(N(c2ccccc2)c2ccccc2C)c2ccccc12)C3(C)C ; Cc1ccccc1N(c1ccccc1C)c1cc2c(c3ccccc13)-c1cc3c(cc1C2(C)C)-c1c(cc(N(c2ccccc2C)c2ccccc2C)c2ccccc12)C3(C)C ; CC(C)(C)c1ccc(N(c2ccc(C(C)(C)C)cc2)c2ccc3c4c(ccc3c2)-c2cc3c(cc2C4(C)C)-c2ccc4cc(N(c5ccc(C(C)(C)C)cc5)c5ccc(C(C)(C)C)cc5)ccc4c2C3(C)C)cc1]

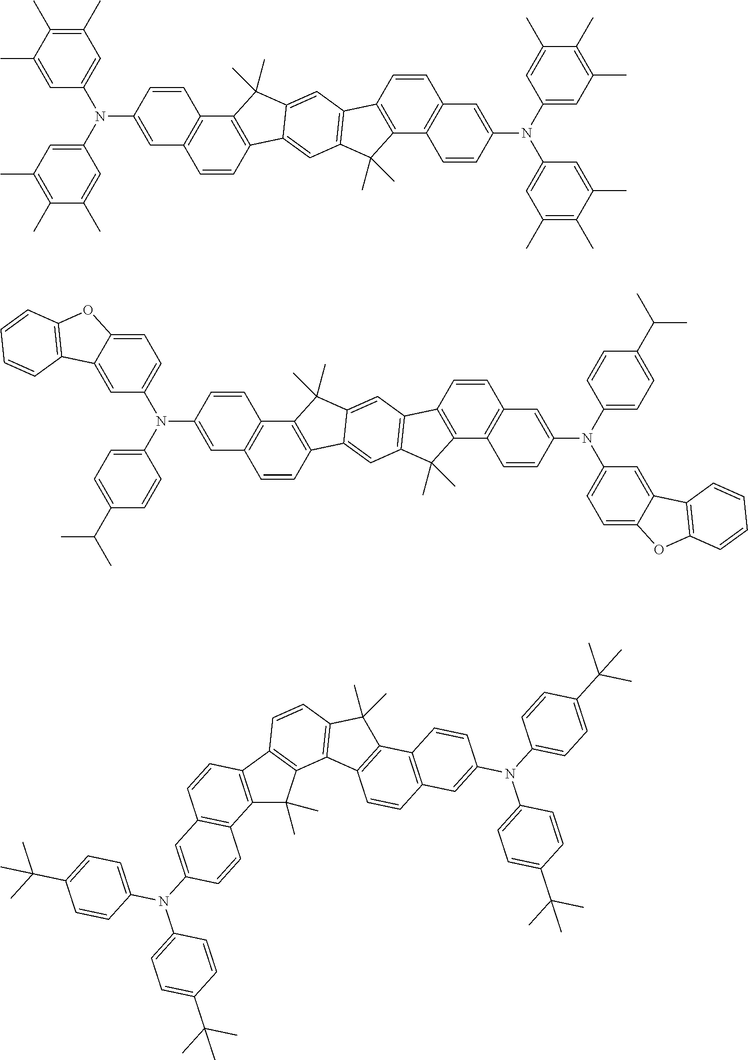

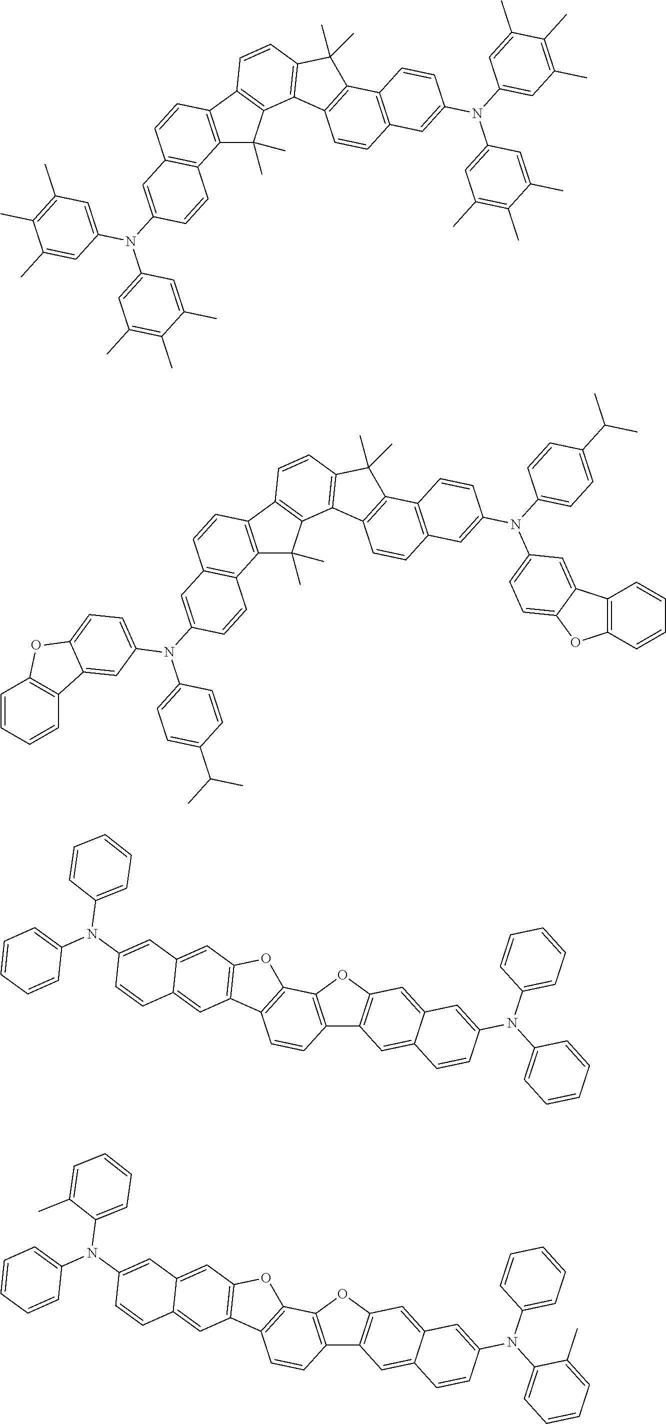

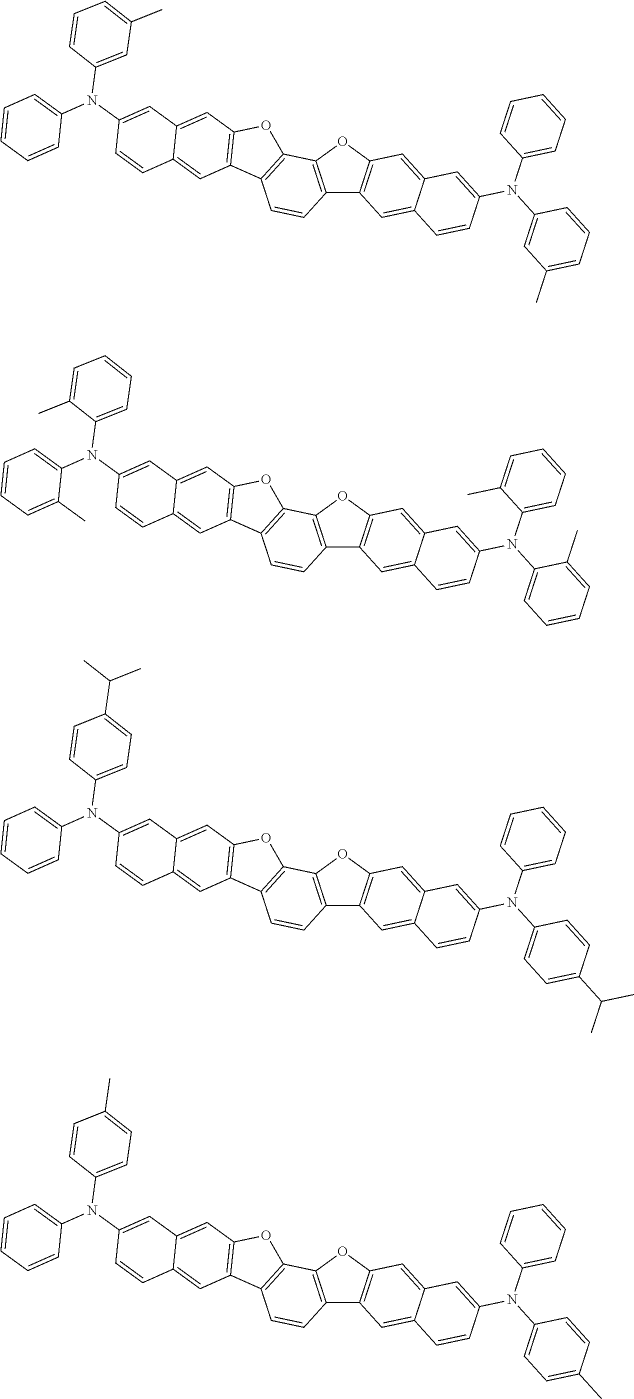 [SMILES: Cc1cccc(N(c2ccccc2)c2ccc3cc4c(cc3c2)oc2c4ccc3c4cc5ccc(N(c6ccccc6)c6cccc(C)c6)cc5cc4oc32)c1 ; Cc1ccccc1N(c1ccc2cc3c(cc2c1)oc1c3ccc2c3cc4ccc(N(c5ccccc5C)c5ccccc5C)cc4cc3oc21)c1ccccc1C ; CC(C)c1ccc(N(c2ccccc2)c2ccc3cc4c(cc3c2)oc2c4ccc3c4cc5ccc(N(c6ccccc6)c6ccc(C(C)C)cc6)cc5cc4oc32)cc1 ; Cc1ccc(N(c2ccccc2)c2ccc3cc4c(cc3c2)oc2c4ccc3c4cc5ccc(N(c6ccccc6)c6ccc(C)cc6)cc5cc4oc32)cc1]

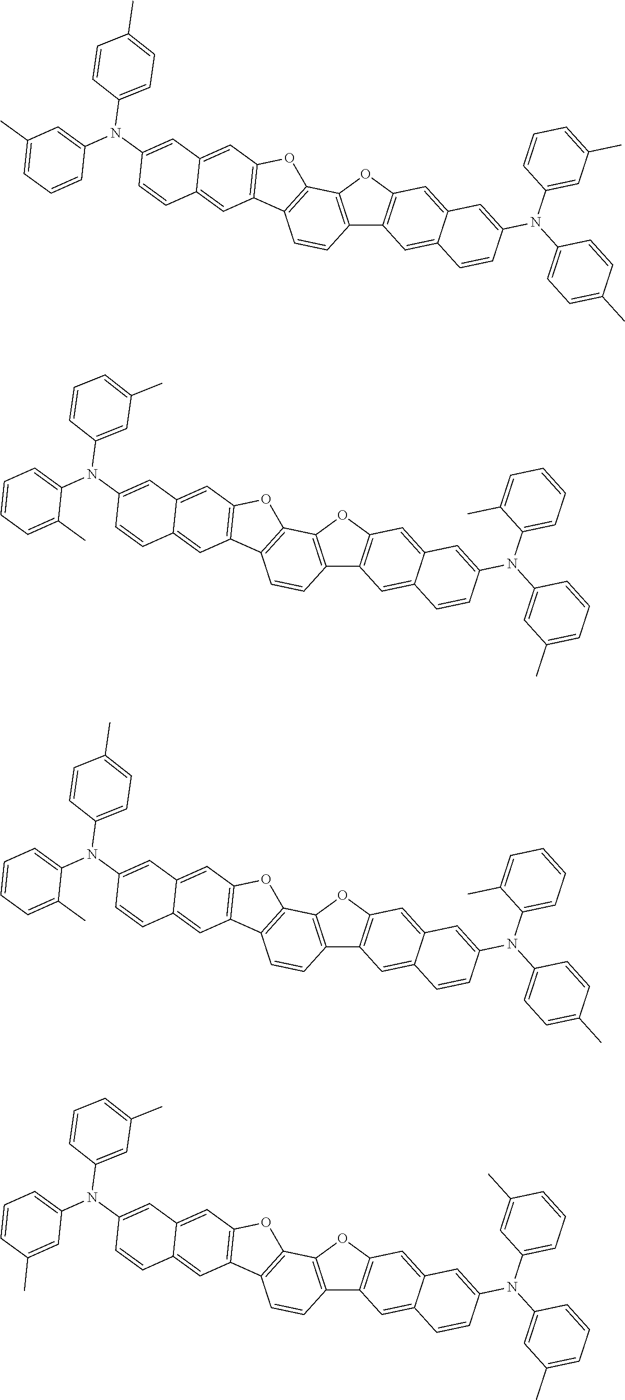

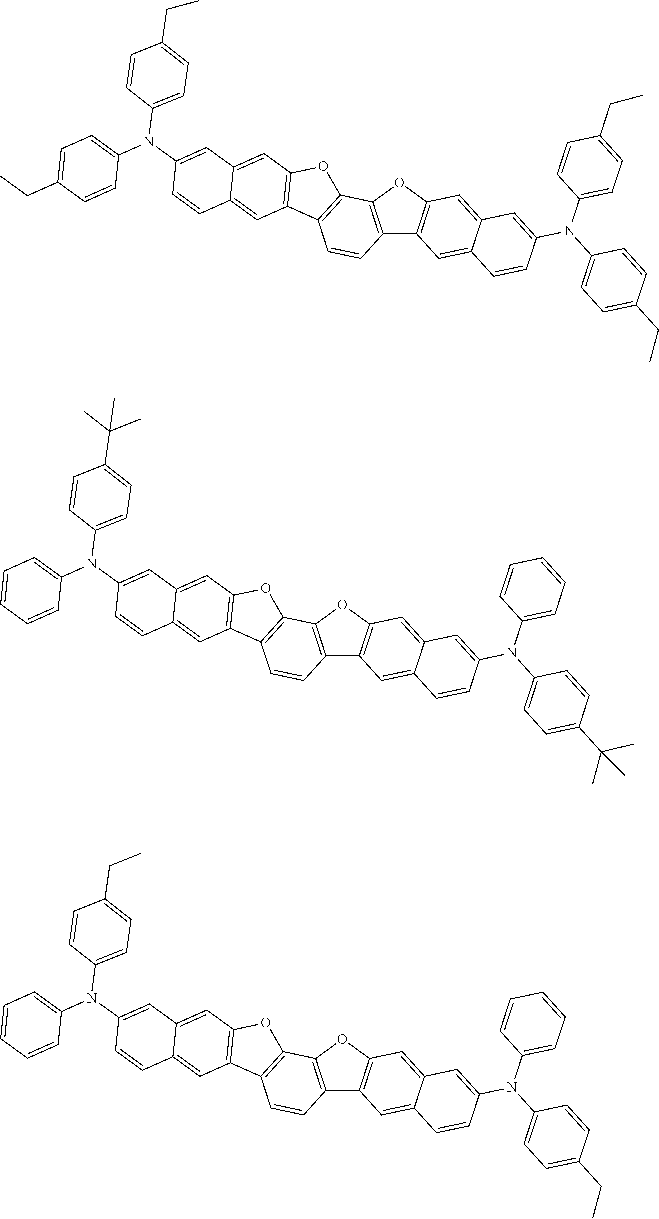 [SMILES: CCc1ccc(N(c2ccc(CC)cc2)c2ccc3cc4c(cc3c2)oc2c4ccc3c4cc5ccc(N(c6ccc(CC)cc6)c6ccc(CC)cc6)cc5cc4oc32)cc1 ; CC(C)(C)c1ccc(N(c2ccccc2)c2ccc3cc4c(cc3c2)oc2c4ccc3c4cc5ccc(N(c6ccccc6)c6ccc(C(C)(C)C)cc6)cc5cc4oc32)cc1 ; CCc1ccc(N(c2ccccc2)c2ccc3cc4c(cc3c2)oc2c4ccc3c4cc5ccc(N(c6ccccc6)c6ccc(CC)cc6)cc5cc4oc32)cc1]

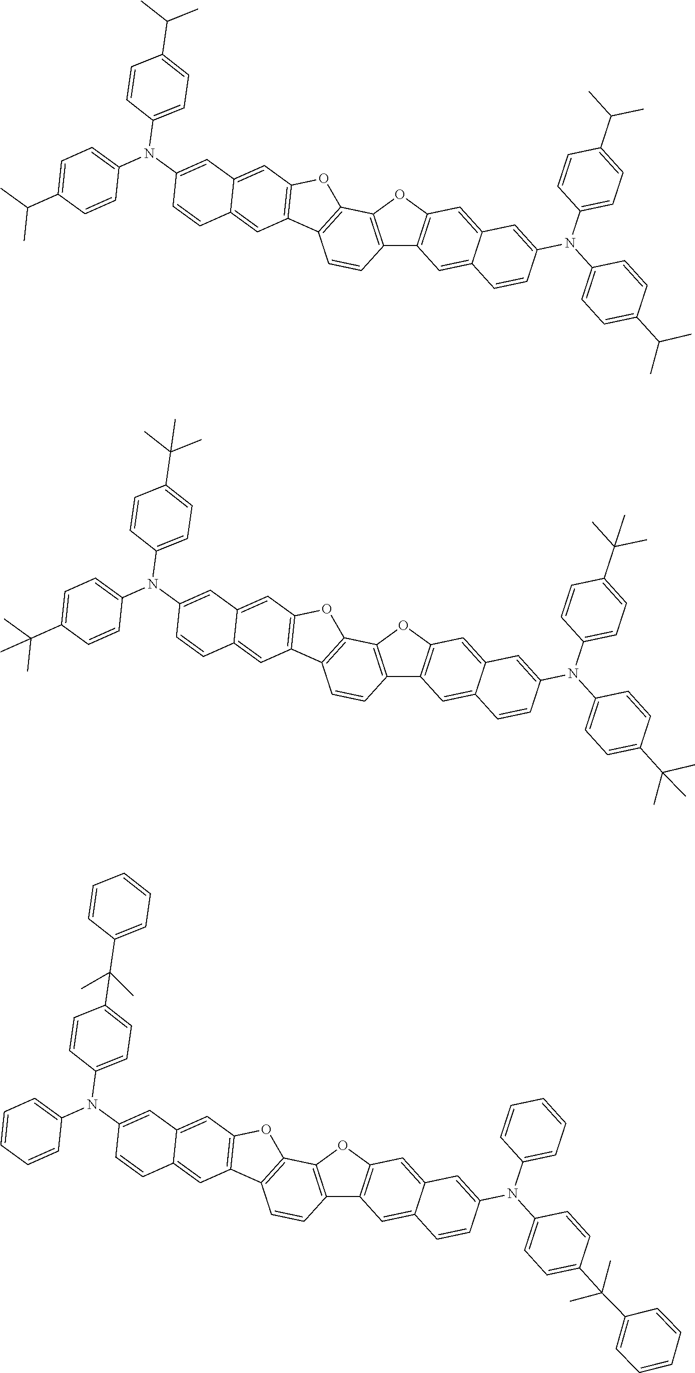

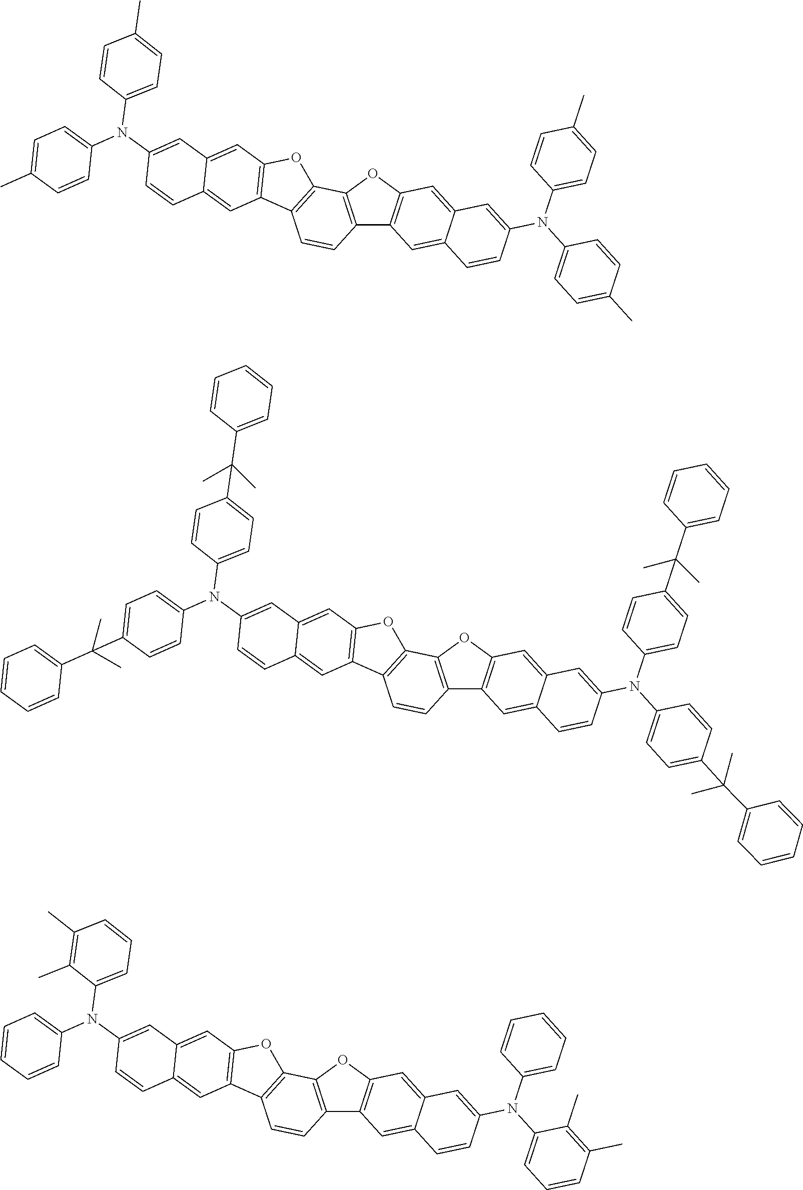

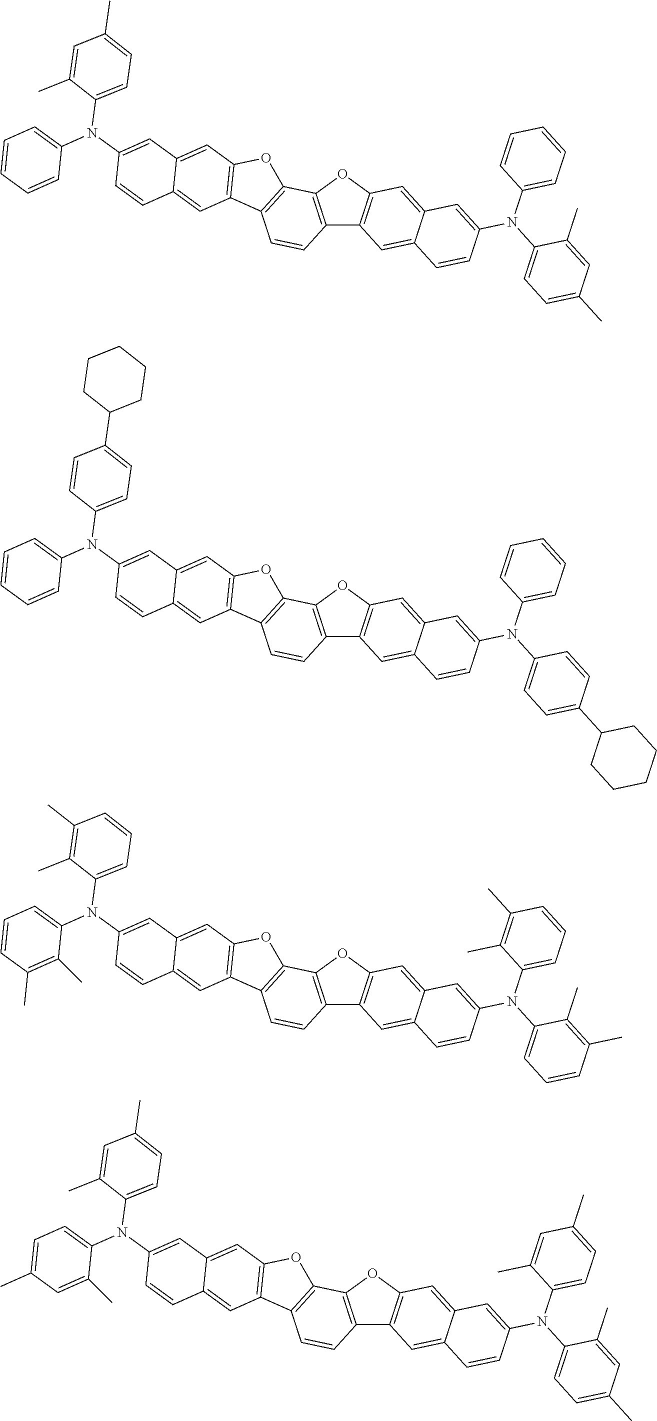

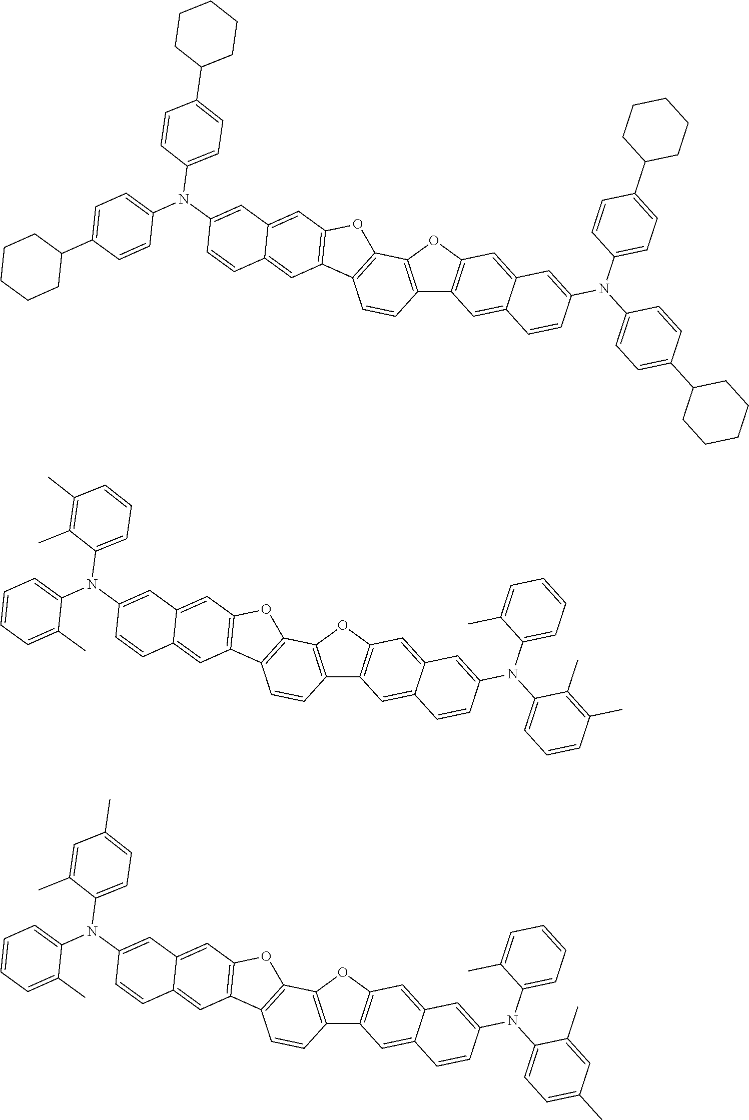 [SMILES: c1cc(N(c2ccc(C3CCCCC3)cc2)c2ccc3cc4c(cc3c2)oc2c4ccc3c4cc5ccc(N(c6ccc(C7CCCCC7)cc6)c6ccc(C7CCCCC7)cc6)cc5cc4oc32)ccc1C1CCCCC1 ; Cc1ccccc1N(c1ccc2cc3c(cc2c1)oc1c3ccc2c3cc4ccc(N(c5ccccc5C)c5cccc(C)c5C)cc4cc3oc21)c1cccc(C)c1C ; Cc1ccc(N(c2ccc3cc4c(cc3c2)oc2c4ccc3c4cc5ccc(N(c6ccccc6C)c6ccc(C)cc6C)cc5cc4oc32)c2ccccc2C)c(C)c1]

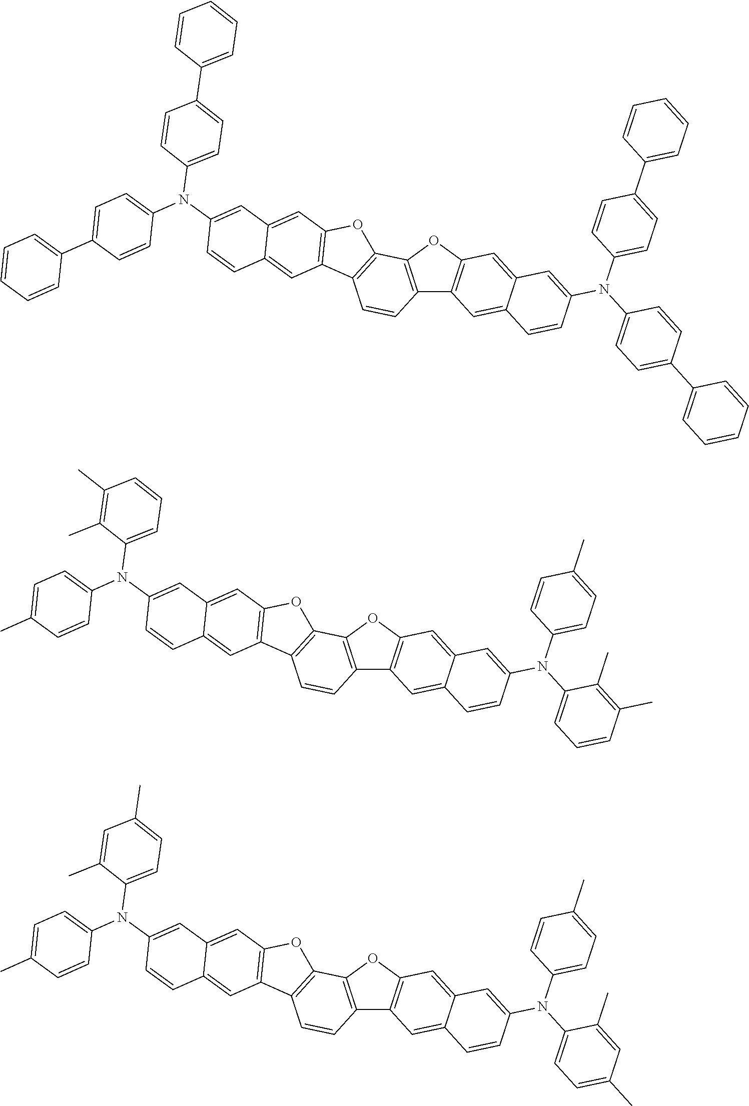

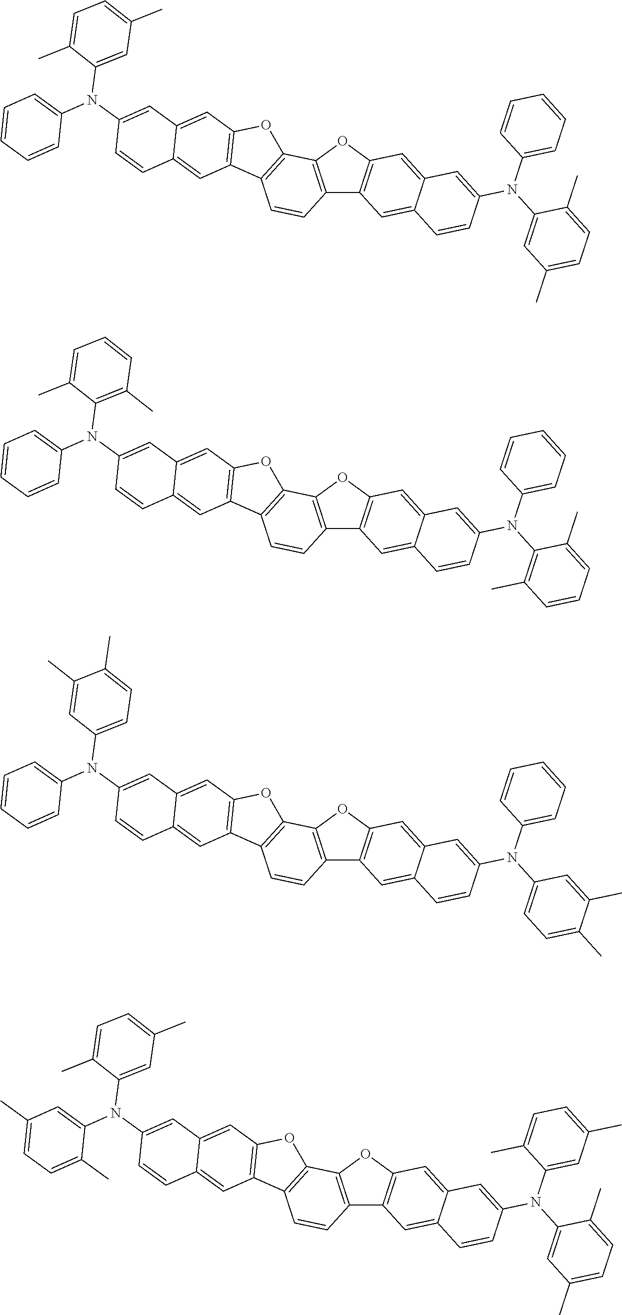

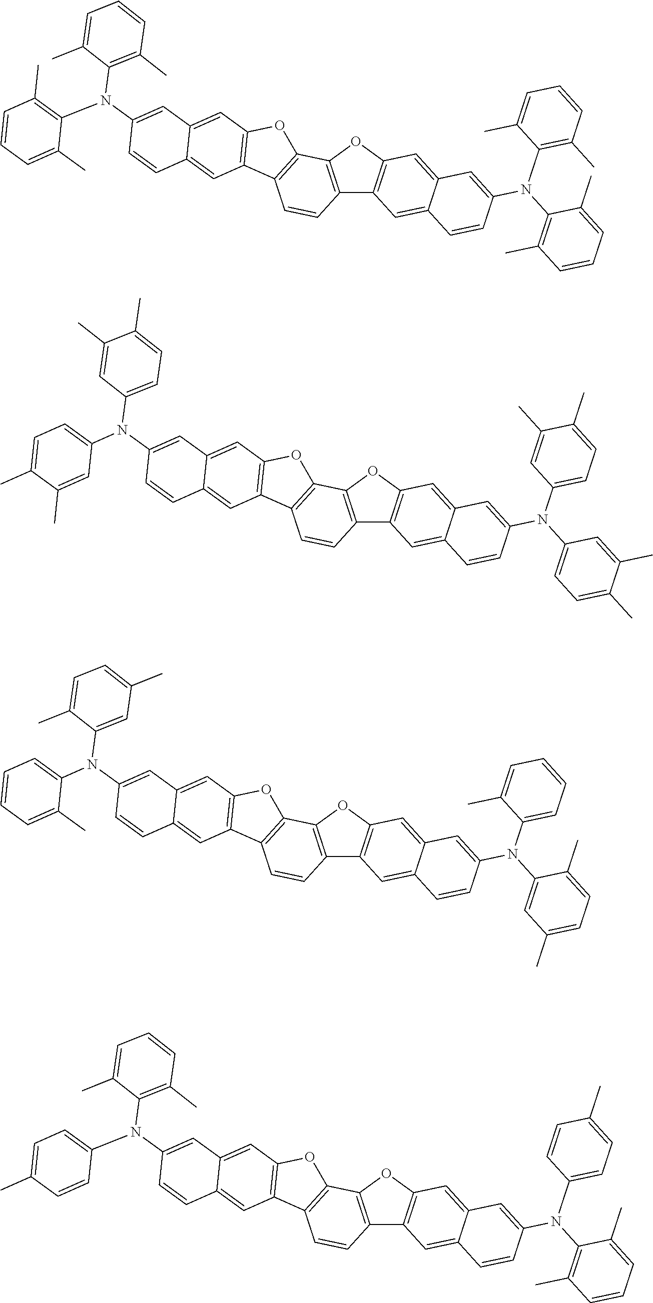

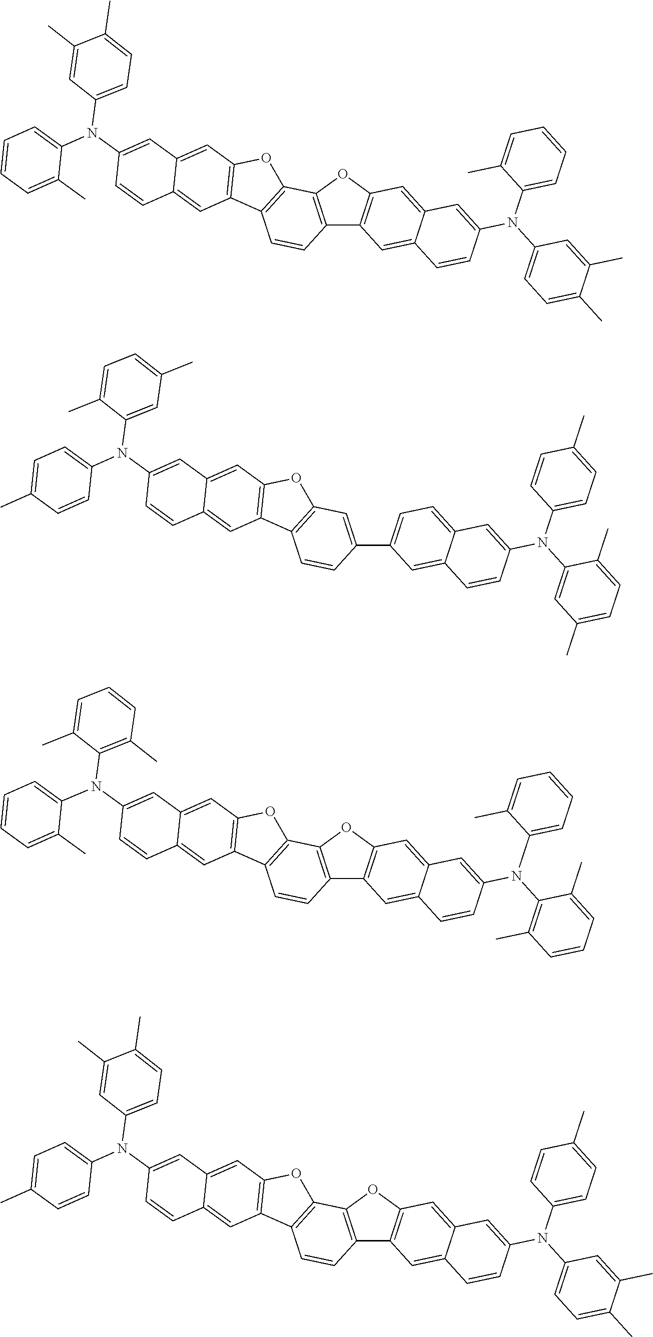

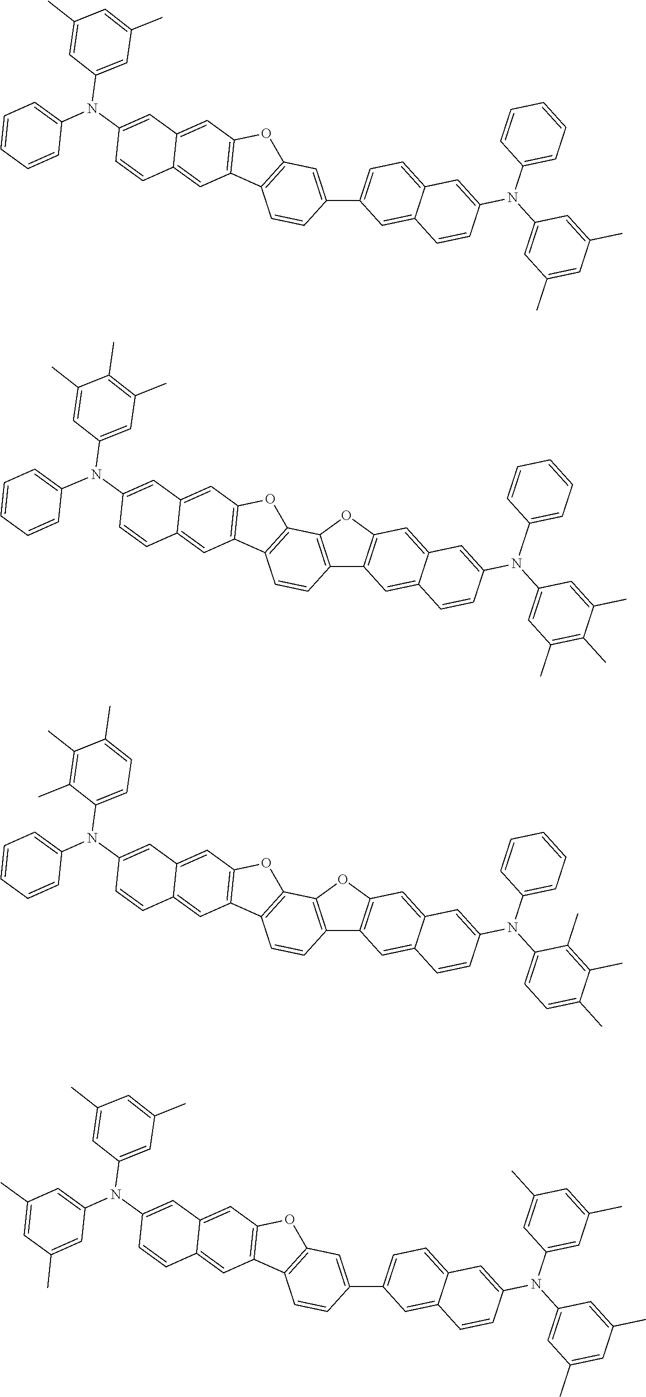

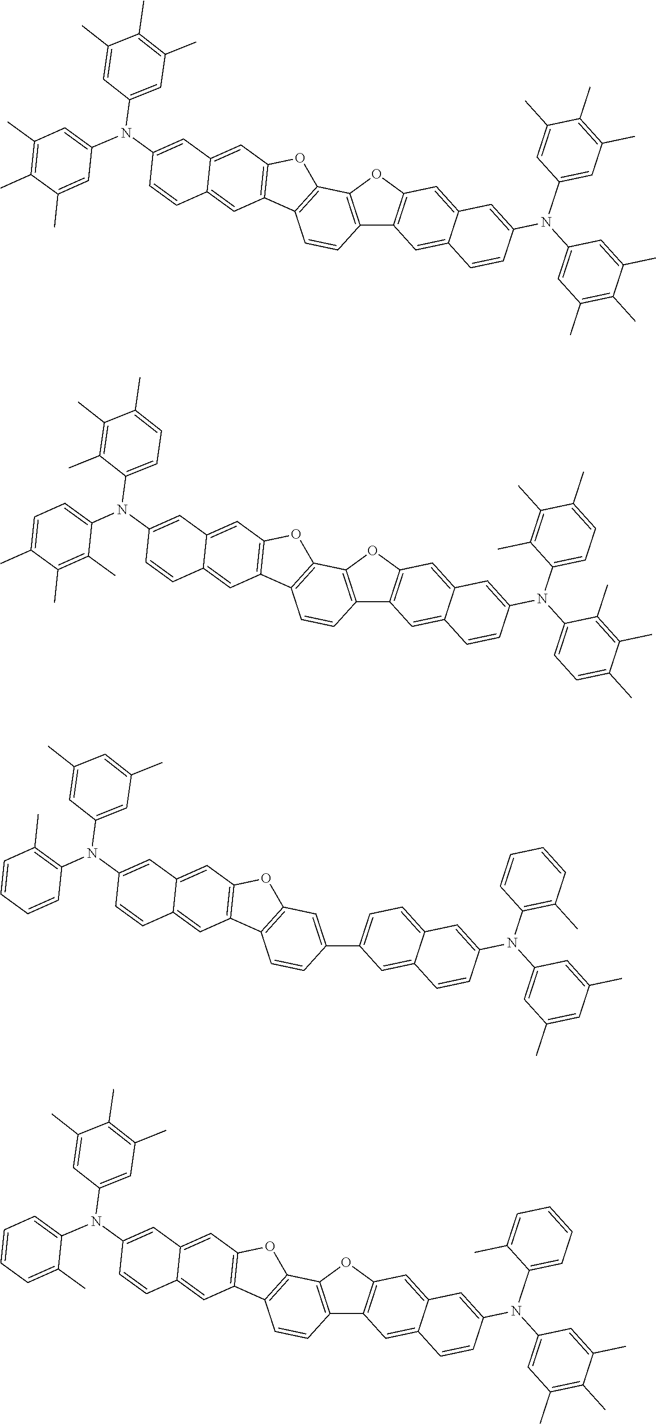

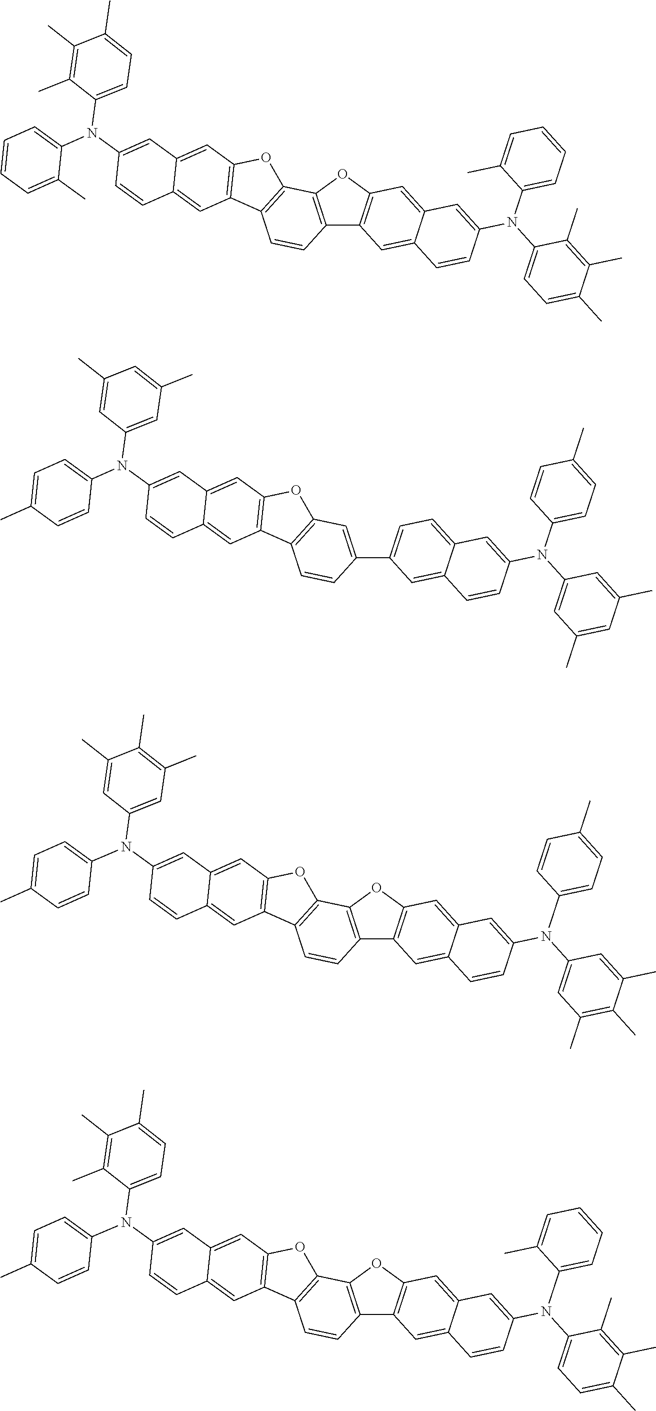

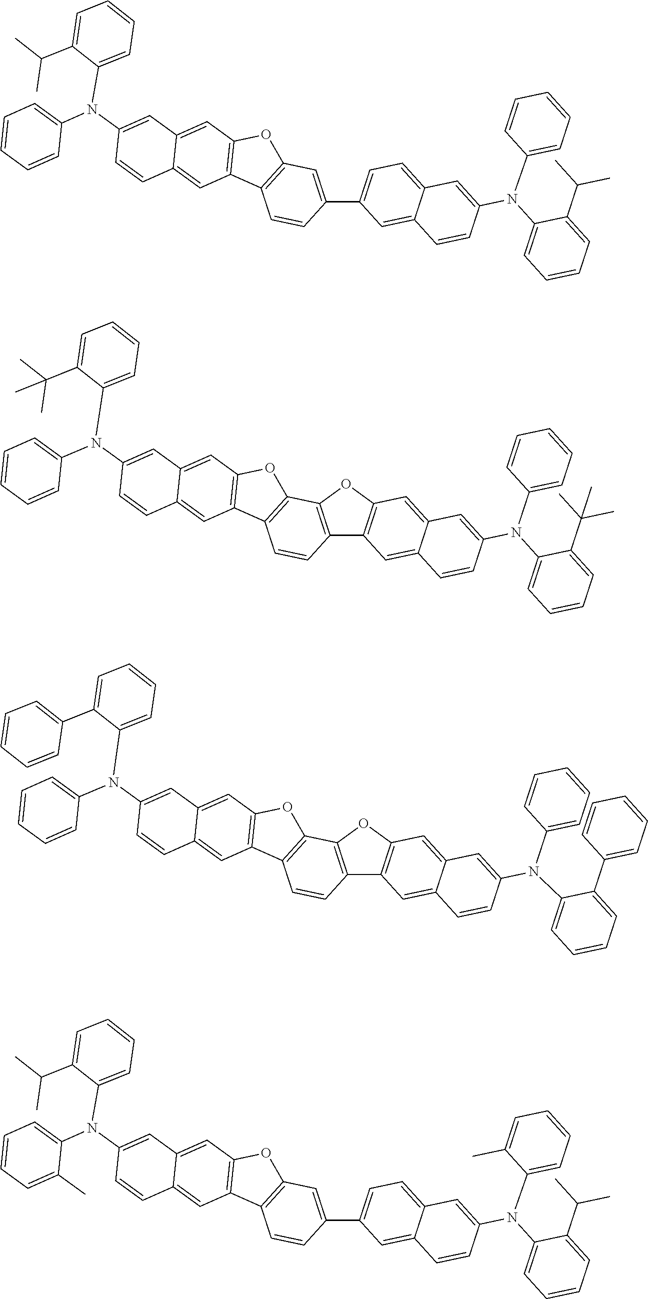 [SMILES: CC(C)c1ccccc1N(c1ccccc1)c1ccc2cc(-c3ccc4c(c3)oc3cc5cc(N(c6ccccc6)c6ccccc6C(C)C)ccc5cc34)ccc2c1 ; CC(C)(C)c1ccccc1N(c1ccccc1)c1ccc2cc3c(cc2c1)oc1c3ccc2c3cc4ccc(N(c5ccccc5)c5ccccc5C(C)(C)C)cc4cc3oc21 ; c1ccc(-c2ccccc2N(c2ccccc2)c2ccc3cc4c(cc3c2)oc2c4ccc3c4cc5ccc(N(c6ccccc6)c6ccccc6-c6ccccc6)cc5cc4oc32)cc1 ; Cc1ccccc1N(c1ccc2cc(-c3ccc4c(c3)oc3cc5cc(N(c6ccccc6C)c6ccccc6C(C)C)ccc5cc34)ccc2c1)c1ccccc1C(C)C]

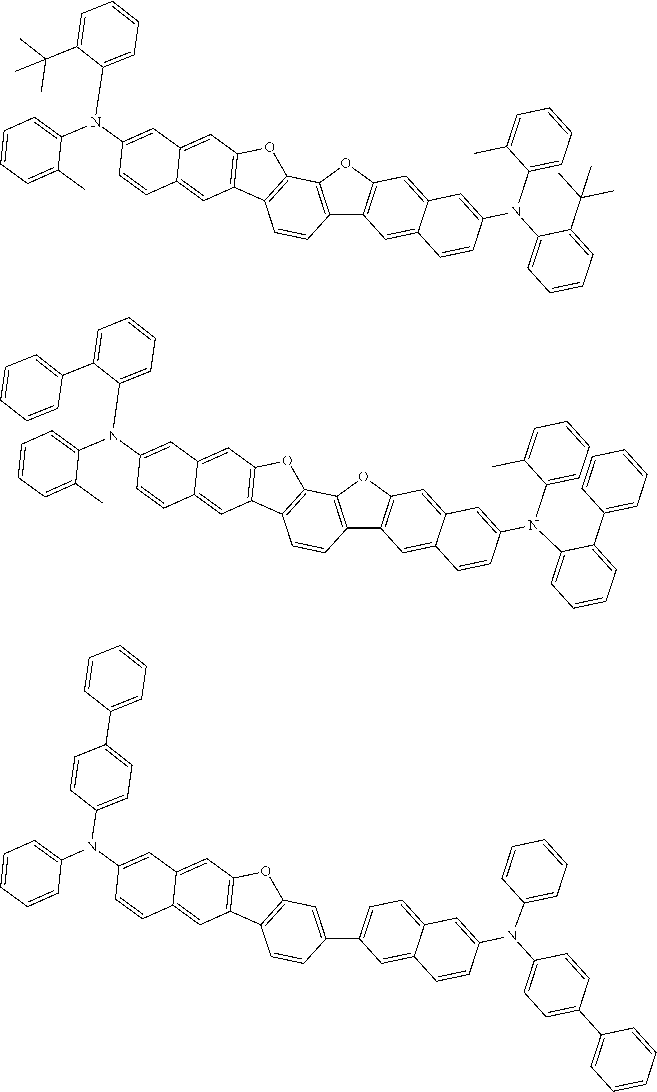 [SMILES: Cc1ccccc1N(c1ccc2cc3c(cc2c1)oc1c3ccc2c3cc4ccc(N(c5ccccc5C)c5ccccc5C(C)(C)C)cc4cc3oc21)c1ccccc1C(C)(C)C ; Cc1ccccc1N(c1ccc2cc3c(cc2c1)oc1c3ccc2c3cc4ccc(N(c5ccccc5C)c5ccccc5-c5ccccc5)cc4cc3oc21)c1ccccc1-c1ccccc1 ; c1ccc(-c2ccc(N(c3ccccc3)c3ccc4cc(-c5ccc6c(c5)oc5cc7cc(N(c8ccccc8)c8ccc(-c9ccccc9)cc8)ccc7cc56)ccc4c3)cc2)cc1]

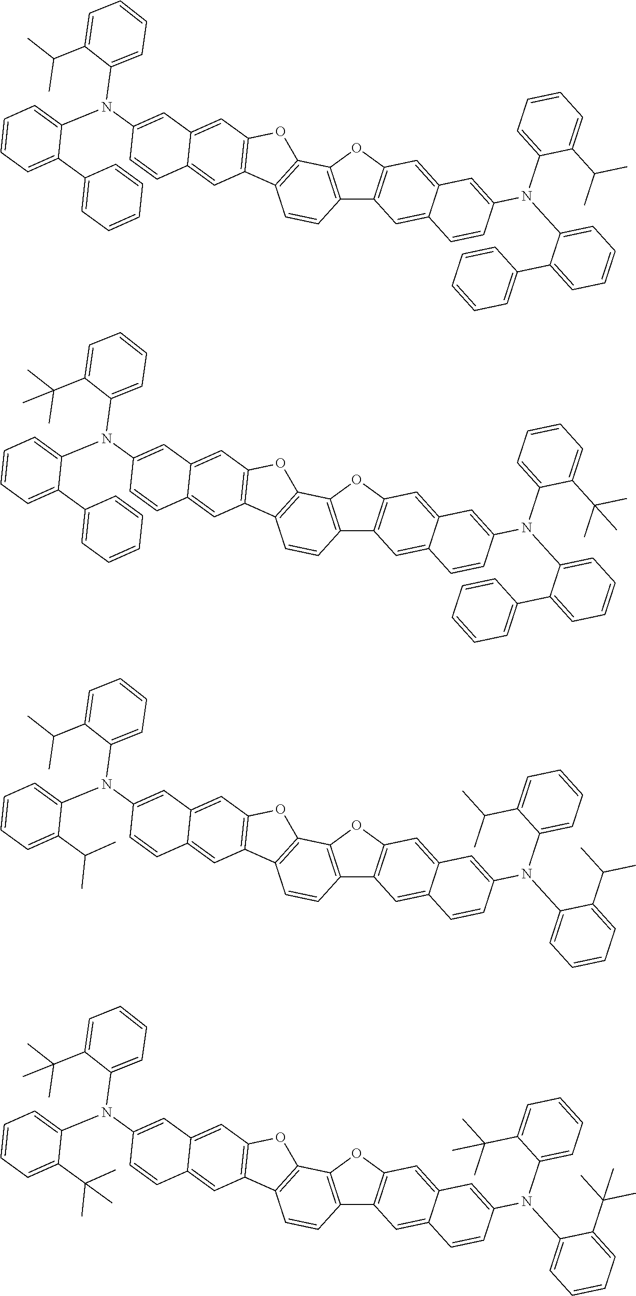 [SMILES: CC(C)c1ccccc1N(c1ccc2cc3c(cc2c1)oc1c3ccc2c3cc4ccc(N(c5ccccc5-c5ccccc5)c5ccccc5C(C)C)cc4cc3oc21)c1ccccc1-c1ccccc1 ; CC(C)(C)c1ccccc1N(c1ccc2cc3c(cc2c1)oc1c3ccc2c3cc4ccc(N(c5ccccc5-c5ccccc5)c5ccccc5C(C)(C)C)cc4cc3oc21)c1ccccc1-c1ccccc1 ; CC(C)c1ccccc1N(c1ccc2cc3c(cc2c1)oc1c3ccc2c3cc4ccc(N(c5ccccc5C(C)C)c5ccccc5C(C)C)cc4cc3oc21)c1ccccc1C(C)C ; CC(C)(C)c1ccccc1N(c1ccc2cc3c(cc2c1)oc1c3ccc2c3cc4ccc(N(c5ccccc5C(C)(C)C)c5ccccc5C(C)(C)C)cc4cc3oc21)c1ccccc1C(C)(C)C]

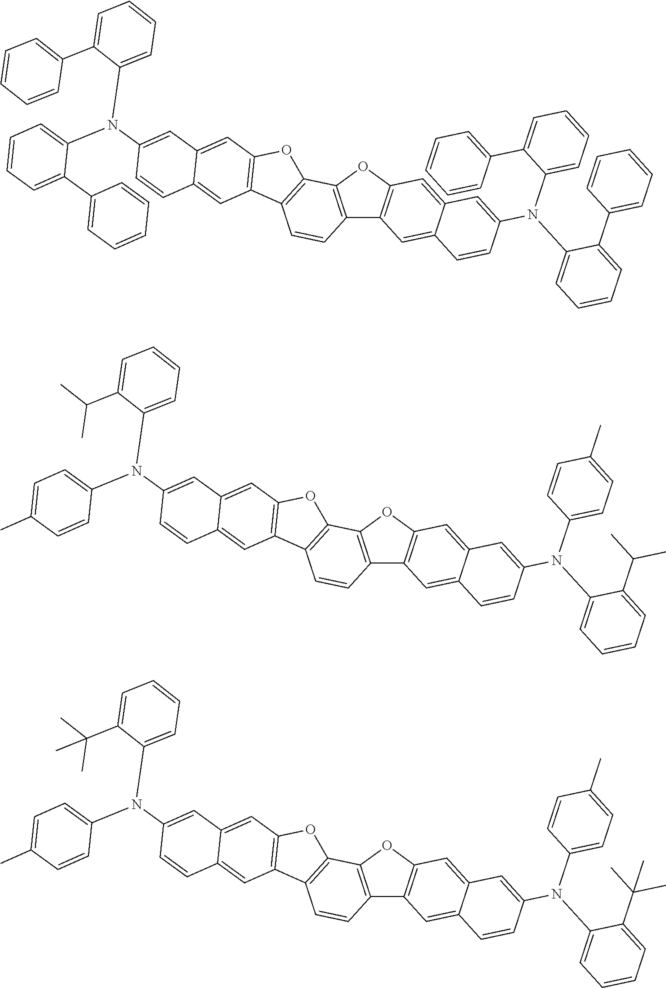

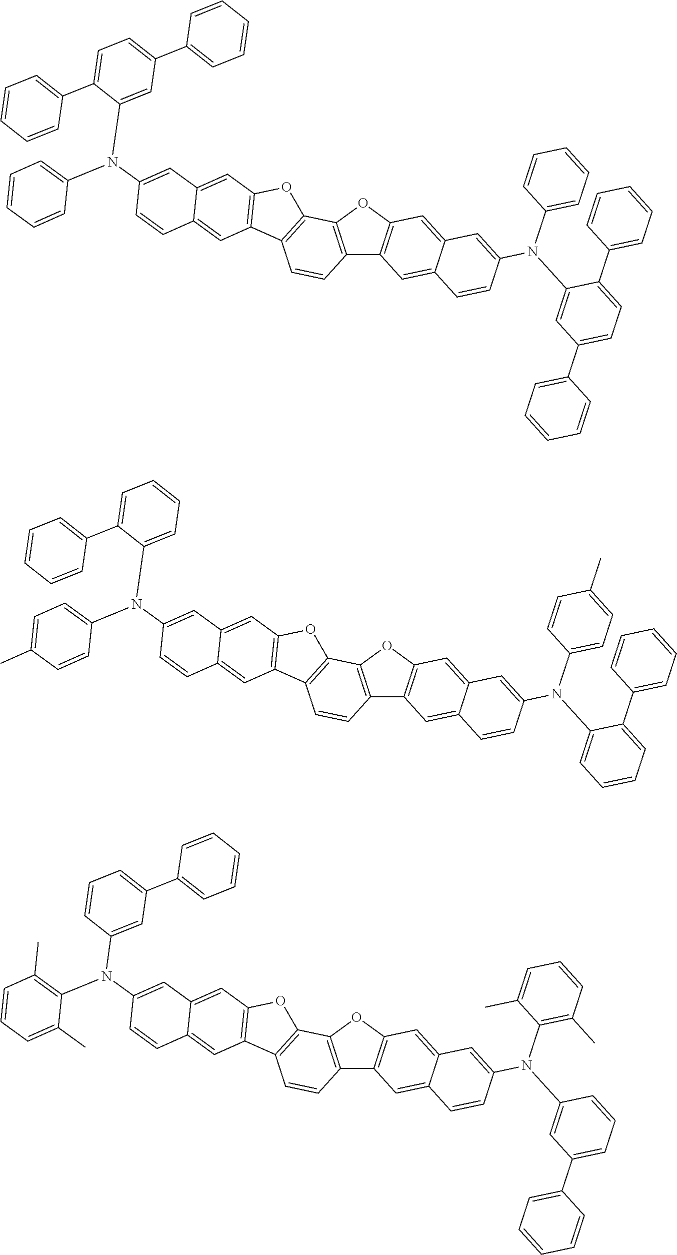 [SMILES: c1ccc(-c2ccc(-c3ccccc3)c(N(c3ccccc3)c3ccc4cc5c(cc4c3)oc3c5ccc4c5cc6ccc(N(c7ccccc7)c7cc(-c8ccccc8)ccc7-c7ccccc7)cc6cc5oc43)c2)cc1 ; Cc1ccc(N(c2ccc3cc4c(cc3c2)oc2c4ccc3c4cc5ccc(N(c6ccc(C)cc6)c6ccccc6-c6ccccc6)cc5cc4oc32)c2ccccc2-c2ccccc2)cc1 ; Cc1cccc(C)c1N(c1cccc(-c2ccccc2)c1)c1ccc2cc3c(cc2c1)oc1c3ccc2c3cc4ccc(N(c5cccc(-c6ccccc6)c5)c5c(C)cccc5C)cc4cc3oc21]

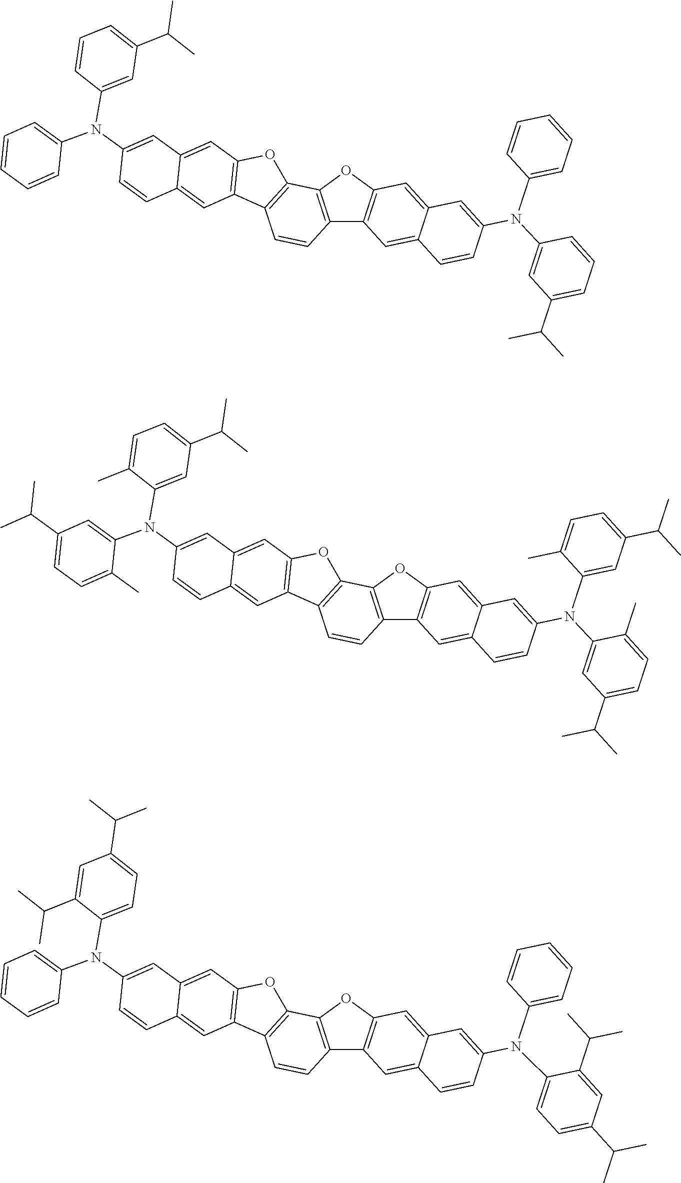 [SMILES: CC(C)c1cccc(N(c2ccccc2)c2ccc3cc4c(cc3c2)oc2c4ccc3c4cc5ccc(N(c6ccccc6)c6cccc(C(C)C)c6)cc5cc4oc32)c1 ; Cc1ccc(C(C)C)cc1N(c1ccc2cc3c(cc2c1)oc1c3ccc2c3cc4ccc(N(c5cc(C(C)C)ccc5C)c5cc(C(C)C)ccc5C)cc4cc3oc21)c1cc(C(C)C)ccc1C ; CC(C)c1ccc(N(c2ccccc2)c2ccc3cc4c(cc3c2)oc2c4ccc3c4cc5ccc(N(c6ccccc6)c6ccc(C(C)C)cc6C(C)C)cc5cc4oc32)c(C(C)C)c1]

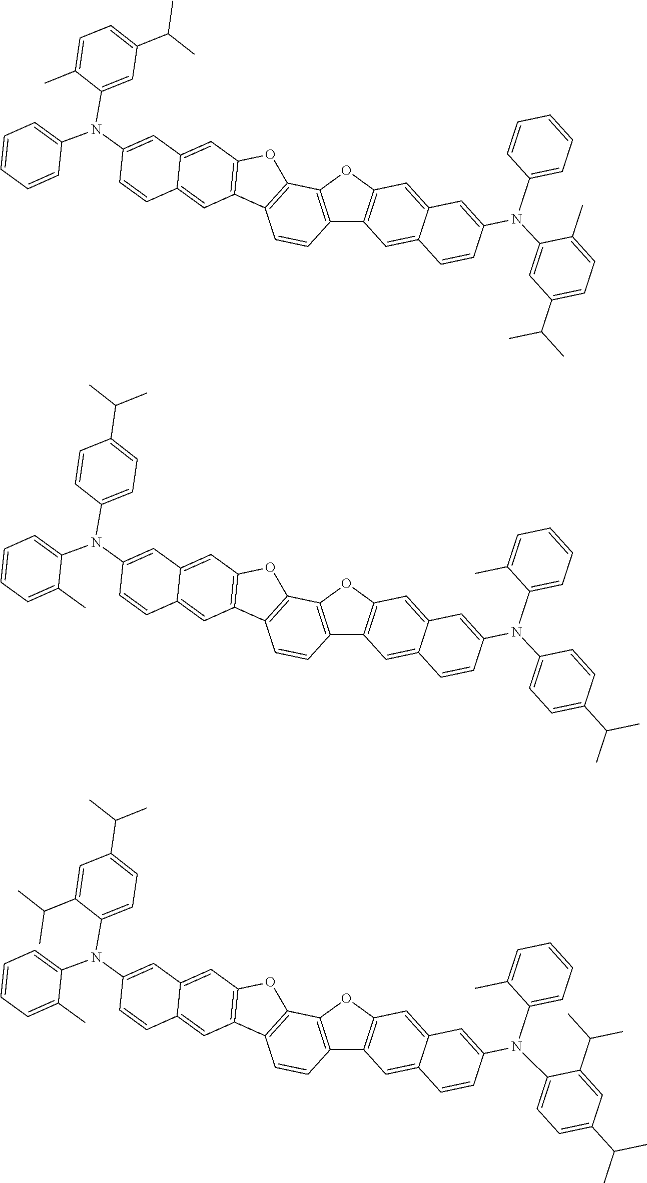 [SMILES: Cc1ccc(C(C)C)cc1N(c1ccccc1)c1ccc2cc3c(cc2c1)oc1c3ccc2c3cc4ccc(N(c5ccccc5)c5cc(C(C)C)ccc5C)cc4cc3oc21 ; Cc1ccccc1N(c1ccc(C(C)C)cc1)c1ccc2cc3c(cc2c1)oc1c3ccc2c3cc4ccc(N(c5ccc(C(C)C)cc5)c5ccccc5C)cc4cc3oc21 ; Cc1ccccc1N(c1ccc2cc3c(cc2c1)oc1c3ccc2c3cc4ccc(N(c5ccccc5C)c5ccc(C(C)C)cc5C(C)C)cc4cc3oc21)c1ccc(C(C)C)cc1C(C)C]

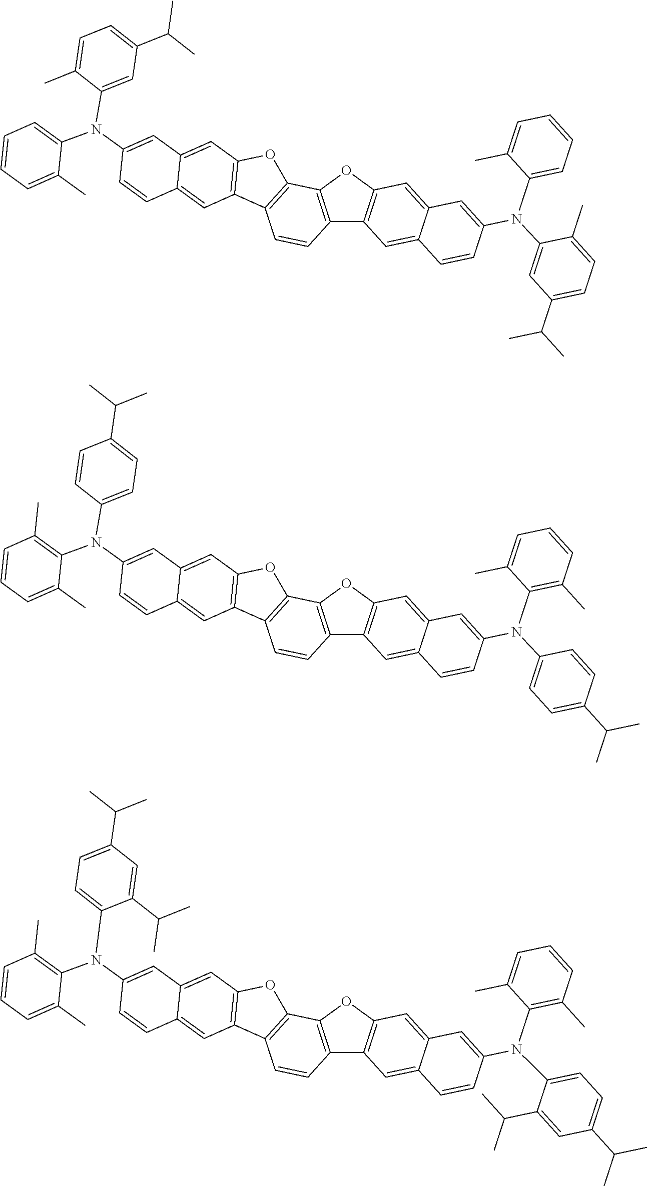 [SMILES: Cc1ccccc1N(c1ccc2cc3c(cc2c1)oc1c3ccc2c3cc4ccc(N(c5ccccc5C)c5cc(C(C)C)ccc5C)cc4cc3oc21)c1cc(C(C)C)ccc1C ; Cc1cccc(C)c1N(c1ccc(C(C)C)cc1)c1ccc2cc3c(cc2c1)oc1c3ccc2c3cc4ccc(N(c5ccc(C(C)C)cc5)c5c(C)cccc5C)cc4cc3oc21 ; Cc1cccc(C)c1N(c1ccc2cc3c(cc2c1)oc1c3ccc2c3cc4ccc(N(c5ccc(C(C)C)cc5C(C)C)c5c(C)cccc5C)cc4cc3oc21)c1ccc(C(C)C)cc1C(C)C]

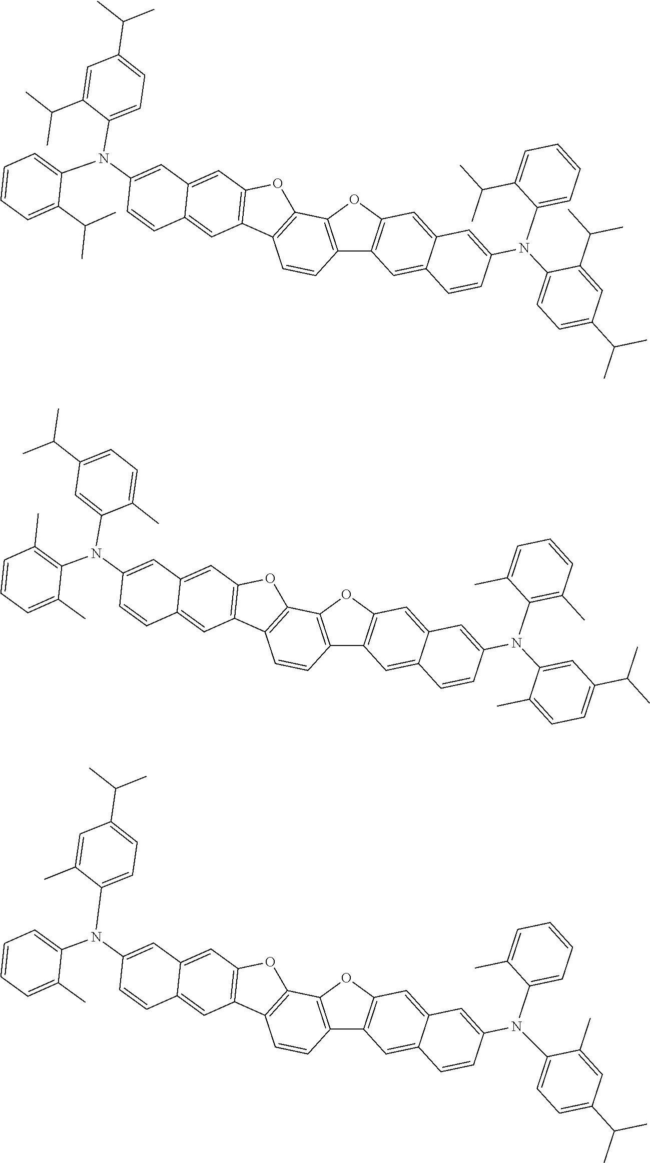 [SMILES: CC(C)c1ccc(N(c2ccc3cc4c(cc3c2)oc2c4ccc3c4cc5ccc(N(c6ccccc6C(C)C)c6ccc(C(C)C)cc6C(C)C)cc5cc4oc32)c2ccccc2C(C)C)c(C(C)C)c1 ; Cc1ccc(C(C)C)cc1N(c1ccc2cc3c(cc2c1)oc1c3ccc2c3cc4ccc(N(c5cc(C(C)C)ccc5C)c5c(C)cccc5C)cc4cc3oc21)c1c(C)cccc1C ; Cc1ccccc1N(c1ccc2cc3c(cc2c1)oc1c3ccc2c3cc4ccc(N(c5ccccc5C)c5ccc(C(C)C)cc5C)cc4cc3oc21)c1ccc(C(C)C)cc1C]

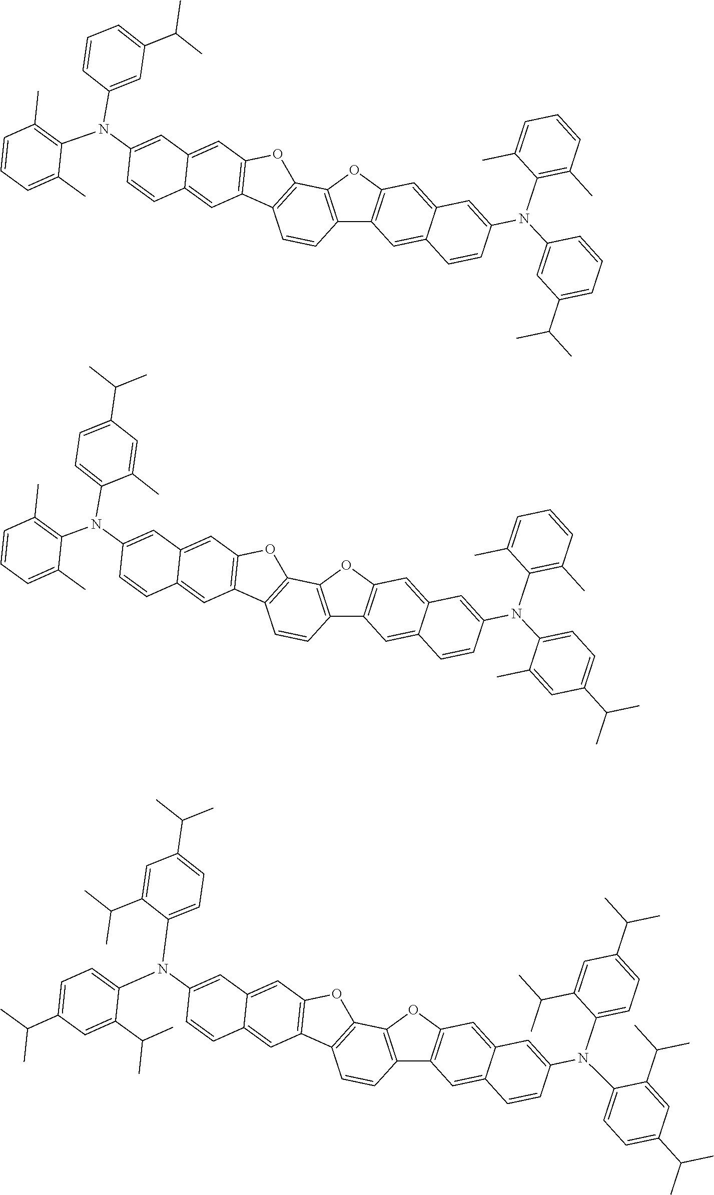

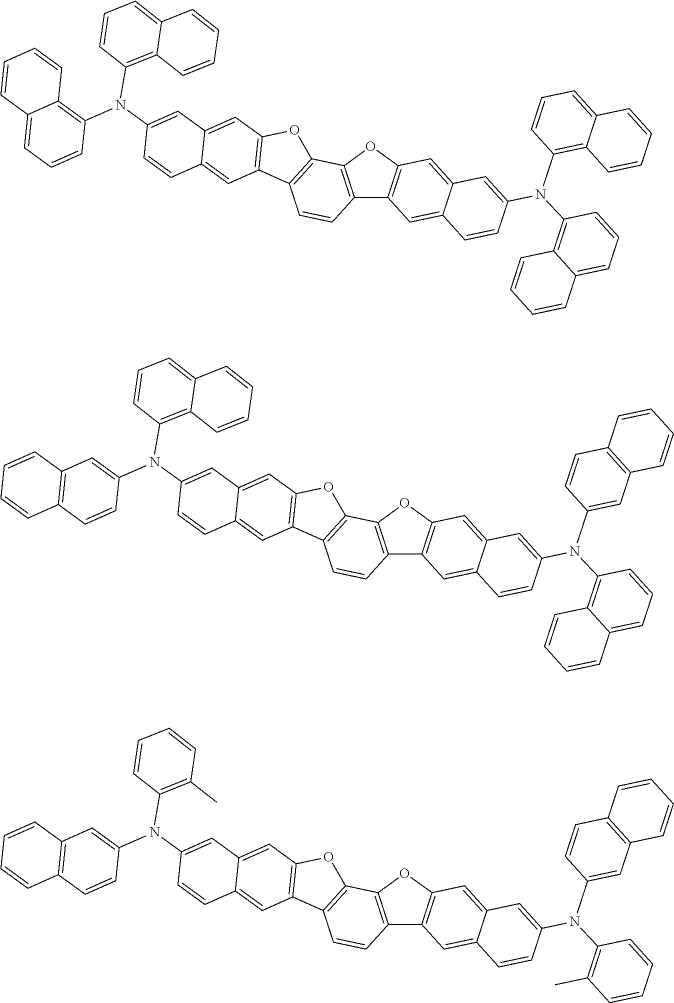 [SMILES: c1ccc2c(N(c3ccc4cc5c(cc4c3)oc3c5ccc4c5cc6ccc(N(c7cccc8ccccc78)c7cccc8ccccc78)cc6cc5oc43)c3cccc4ccccc34)cccc2c1 ; c1ccc2cc(N(c3ccc4cc5c(cc4c3)oc3c5ccc4c5cc6ccc(N(c7ccc8ccccc8c7)c7cccc8ccccc78)cc6cc5oc43)c3cccc4ccccc34)ccc2c1 ; Cc1ccccc1N(c1ccc2ccccc2c1)c1ccc2cc3c(cc2c1)oc1c3ccc2c3cc4ccc(N(c5ccc6ccccc6c5)c5ccccc5C)cc4cc3oc21]

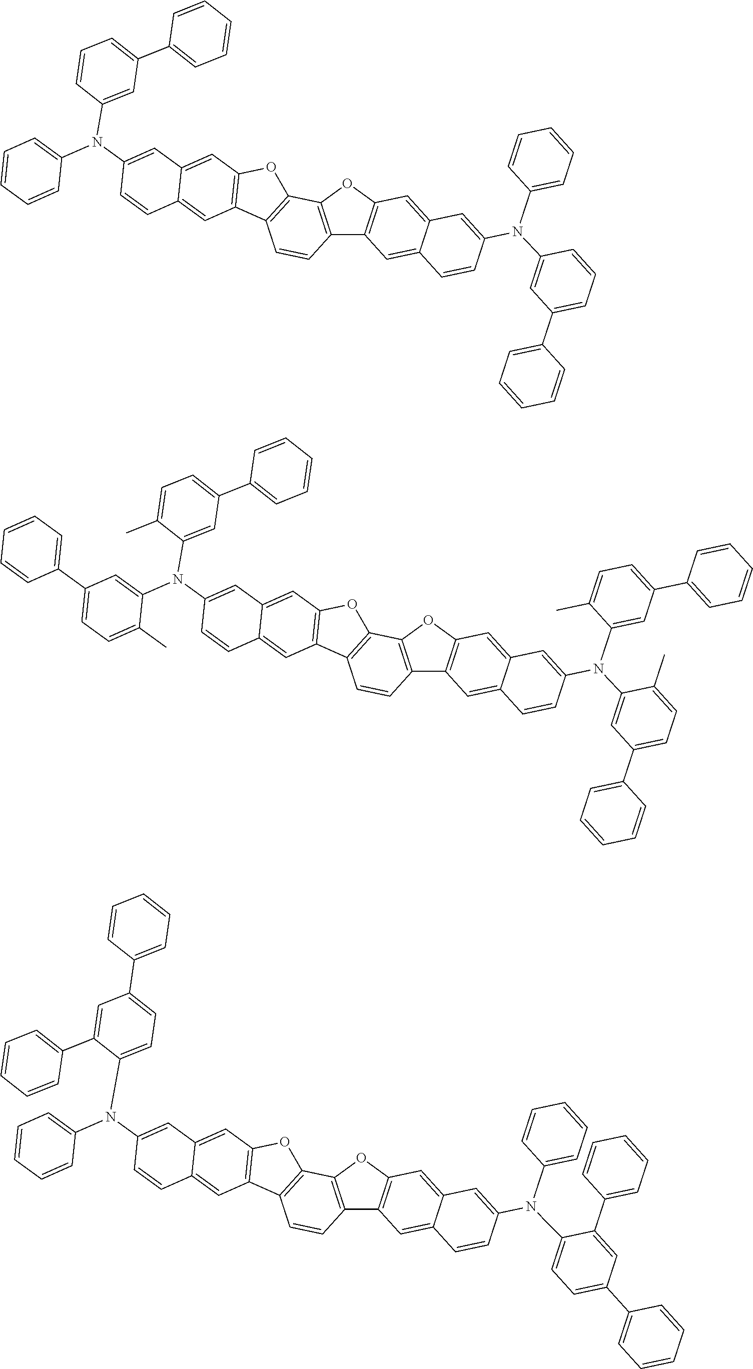 [SMILES: c1ccc(-c2cccc(N(c3ccccc3)c3ccc4cc5c(cc4c3)oc3c5ccc4c5cc6ccc(N(c7ccccc7)c7cccc(-c8ccccc8)c7)cc6cc5oc43)c2)cc1 ; Cc1ccc(-c2ccccc2)cc1N(c1ccc2cc3c(cc2c1)oc1c3ccc2c3cc4ccc(N(c5cc(-c6ccccc6)ccc5C)c5cc(-c6ccccc6)ccc5C)cc4cc3oc21)c1cc(-c2ccccc2)ccc1C ; c1ccc(-c2ccc(N(c3ccccc3)c3ccc4cc5c(cc4c3)oc3c5ccc4c5cc6ccc(N(c7ccccc7)c7ccc(-c8ccccc8)cc7-c7ccccc7)cc6cc5oc43)c(-c3ccccc3)c2)cc1]

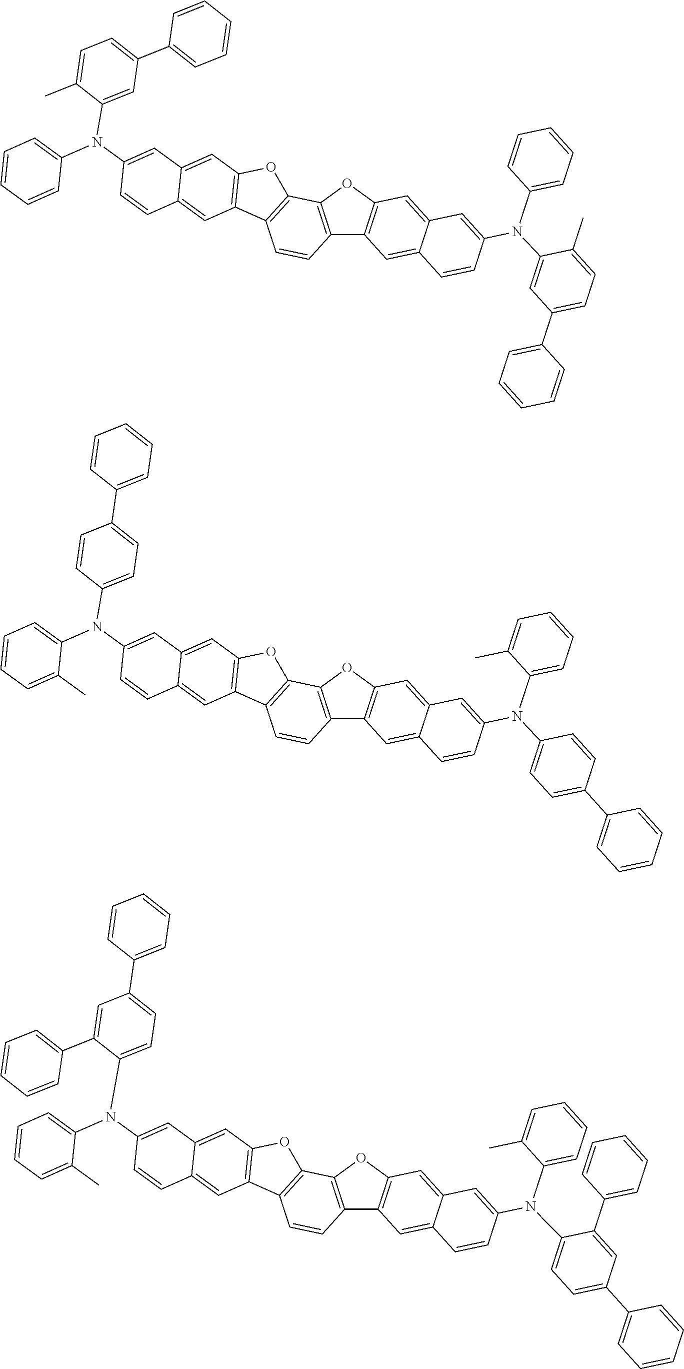 [SMILES: Cc1ccc(-c2ccccc2)cc1N(c1ccccc1)c1ccc2cc3c(cc2c1)oc1c3ccc2c3cc4ccc(N(c5ccccc5)c5cc(-c6ccccc6)ccc5C)cc4cc3oc21 ; Cc1ccccc1N(c1ccc(-c2ccccc2)cc1)c1ccc2cc3c(cc2c1)oc1c3ccc2c3cc4ccc(N(c5ccc(-c6ccccc6)cc5)c5ccccc5C)cc4cc3oc21 ; Cc1ccccc1N(c1ccc2cc3c(cc2c1)oc1c3ccc2c3cc4ccc(N(c5ccccc5C)c5ccc(-c6ccccc6)cc5-c5ccccc5)cc4cc3oc21)c1ccc(-c2ccccc2)cc1-c1ccccc1]

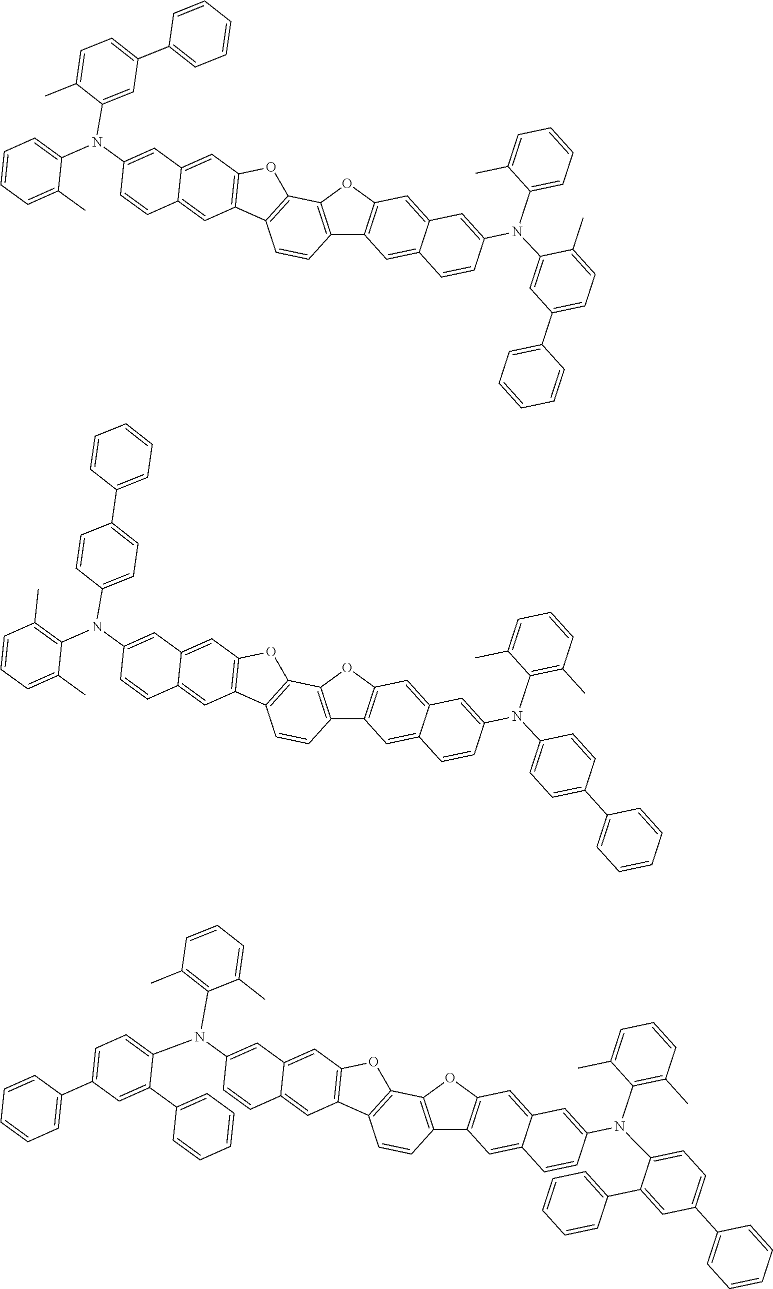 [SMILES: Cc1ccccc1N(c1ccc2cc3c(cc2c1)oc1c3ccc2c3cc4ccc(N(c5ccccc5C)c5cc(-c6ccccc6)ccc5C)cc4cc3oc21)c1cc(-c2ccccc2)ccc1C ; Cc1cccc(C)c1N(c1ccc(-c2ccccc2)cc1)c1ccc2cc3c(cc2c1)oc1c3ccc2c3cc4ccc(N(c5ccc(-c6ccccc6)cc5)c5c(C)cccc5C)cc4cc3oc21 ; Cc1cccc(C)c1N(c1ccc2cc3c(cc2c1)oc1c3ccc2c3cc4ccc(N(c5ccc(-c6ccccc6)cc5-c5ccccc5)c5c(C)cccc5C)cc4cc3oc21)c1ccc(-c2ccccc2)cc1-c1ccccc1]

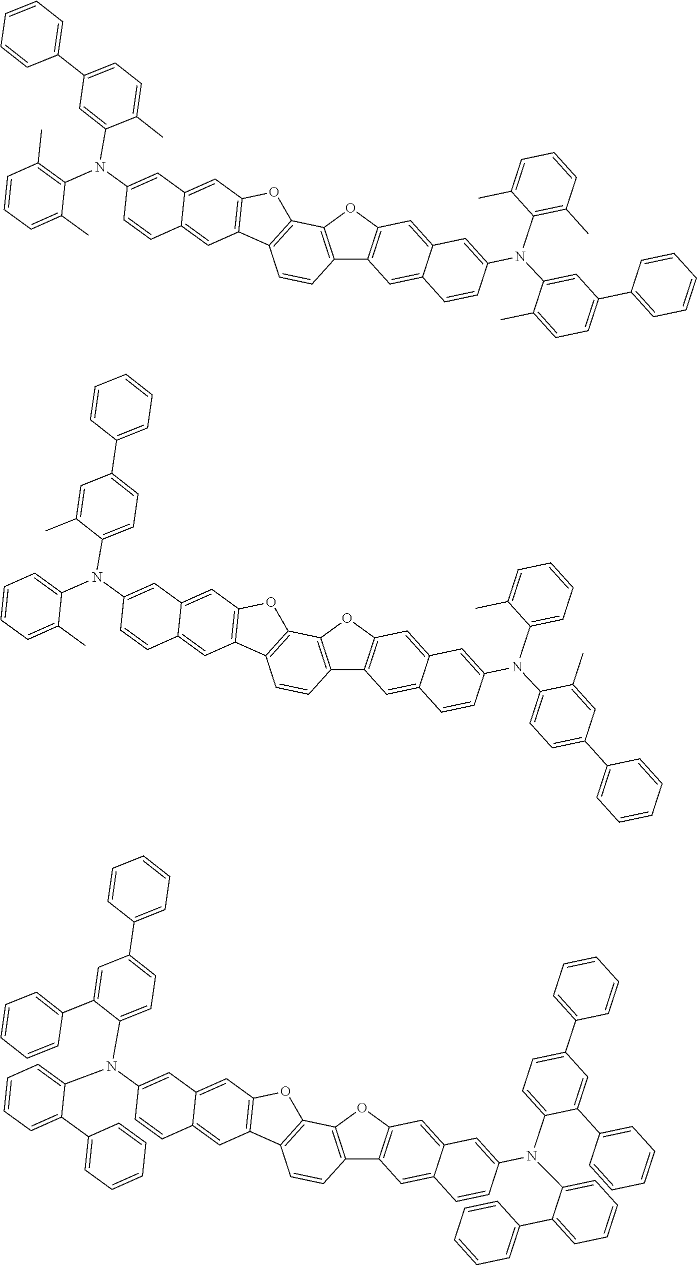 [SMILES: Cc1ccc(-c2ccccc2)cc1N(c1ccc2cc3c(cc2c1)oc1c3ccc2c3cc4ccc(N(c5cc(-c6ccccc6)ccc5C)c5c(C)cccc5C)cc4cc3oc21)c1c(C)cccc1C ; Cc1ccccc1N(c1ccc2cc3c(cc2c1)oc1c3ccc2c3cc4ccc(N(c5ccccc5C)c5ccc(-c6ccccc6)cc5C)cc4cc3oc21)c1ccc(-c2ccccc2)cc1C ; c1ccc(-c2ccc(N(c3ccc4cc5c(cc4c3)oc3c5ccc4c5cc6ccc(N(c7ccccc7-c7ccccc7)c7ccc(-c8ccccc8)cc7-c7ccccc7)cc6cc5oc43)c3ccccc3-c3ccccc3)c(-c3ccccc3)c2)cc1]

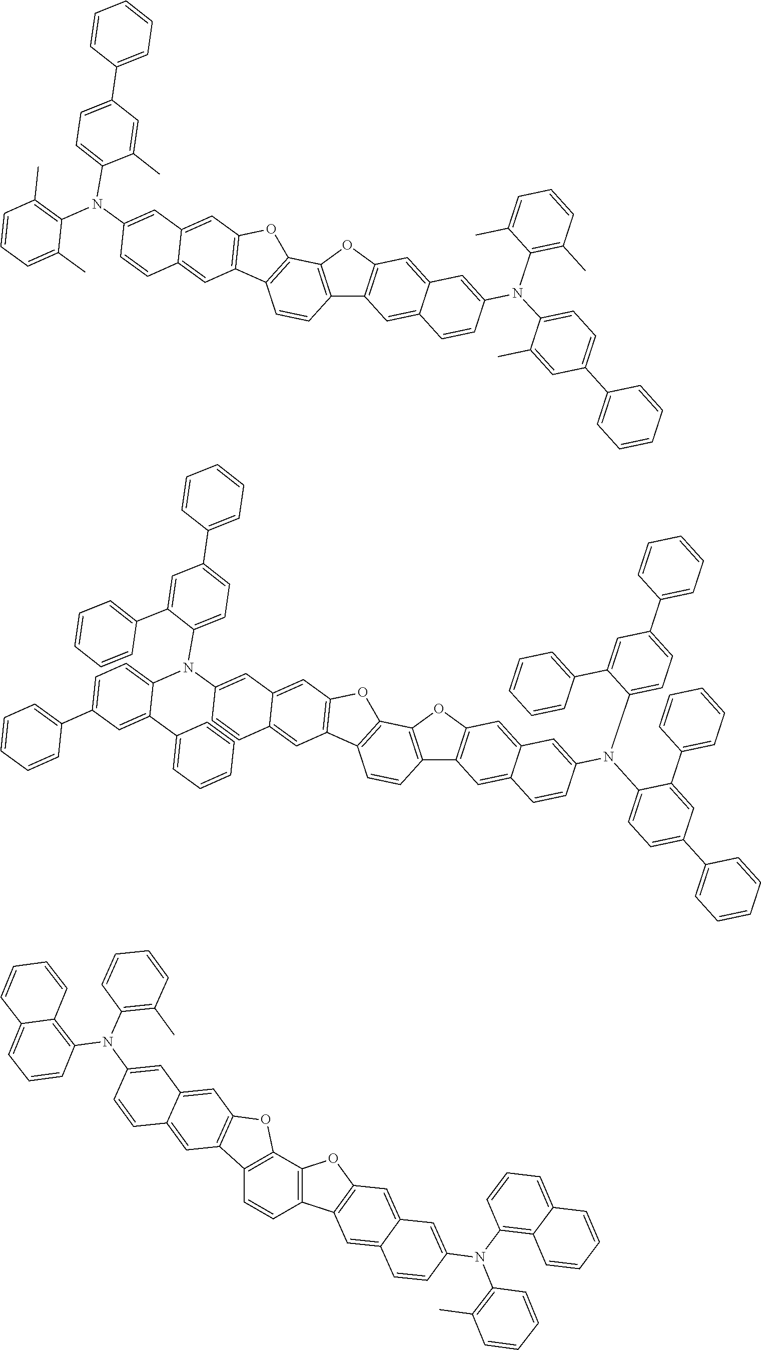 [SMILES: Cc1cc(-c2ccccc2)ccc1N(c1ccc2cc3c(cc2c1)oc1c3ccc2c3cc4ccc(N(c5ccc(-c6ccccc6)cc5C)c5c(C)cccc5C)cc4cc3oc21)c1c(C)cccc1C ; c1ccc(-c2ccc(N(c3ccc4cc5c(cc4c3)oc3c5ccc4c5cc6ccc(N(c7ccc(-c8ccccc8)cc7-c7ccccc7)c7ccc(-c8ccccc8)cc7-c7ccccc7)cc6cc5oc43)c3ccc(-c4ccccc4)cc3-c3ccccc3)c(-c3ccccc3)c2)cc1 ; Cc1ccccc1N(c1ccc2cc3c(cc2c1)oc1c3ccc2c3cc4ccc(N(c5ccccc5C)c5cccc6ccccc56)cc4cc3oc21)c1cccc2ccccc12]

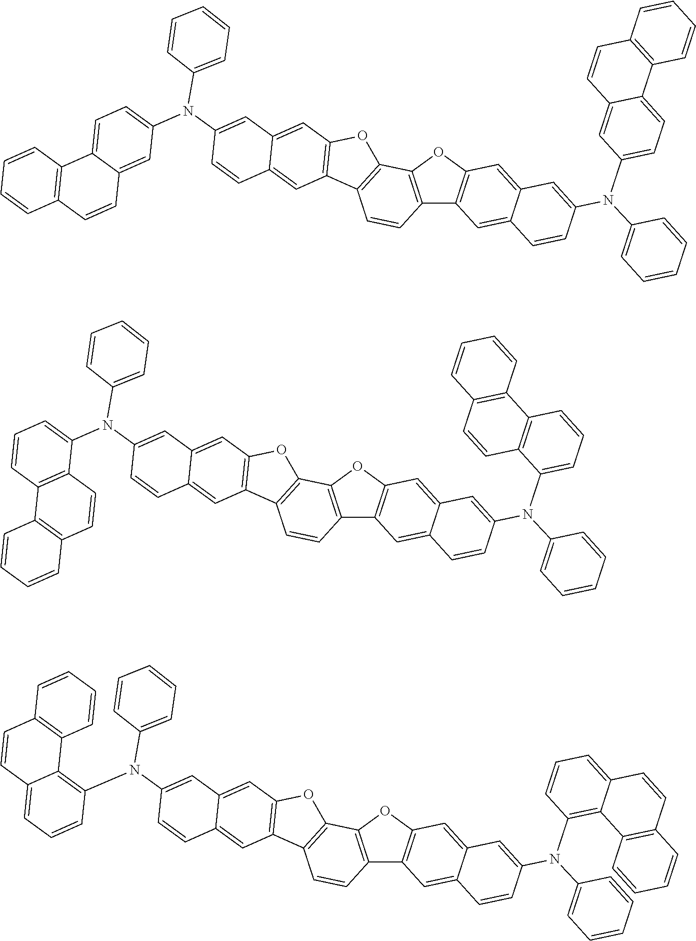 [SMILES: c1ccc(N(c2ccc3cc4c(cc3c2)oc2c4ccc3c4cc5ccc(N(c6ccccc6)c6ccc7c(ccc8ccccc87)c6)cc5cc4oc32)c2ccc3c(ccc4ccccc43)c2)cc1 ; c1ccc(N(c2ccc3cc4c(cc3c2)oc2c4ccc3c4cc5ccc(N(c6ccccc6)c6cccc7c6ccc6ccccc67)cc5cc4oc32)c2cccc3c2ccc2ccccc23)cc1 ; c1ccc(N(c2ccc3cc4c(cc3c2)oc2c4ccc3c4cc5ccc(N(c6ccccc6)c6cccc7ccc8ccccc8c67)cc5cc4oc32)c2cccc3ccc4ccccc4c23)cc1]

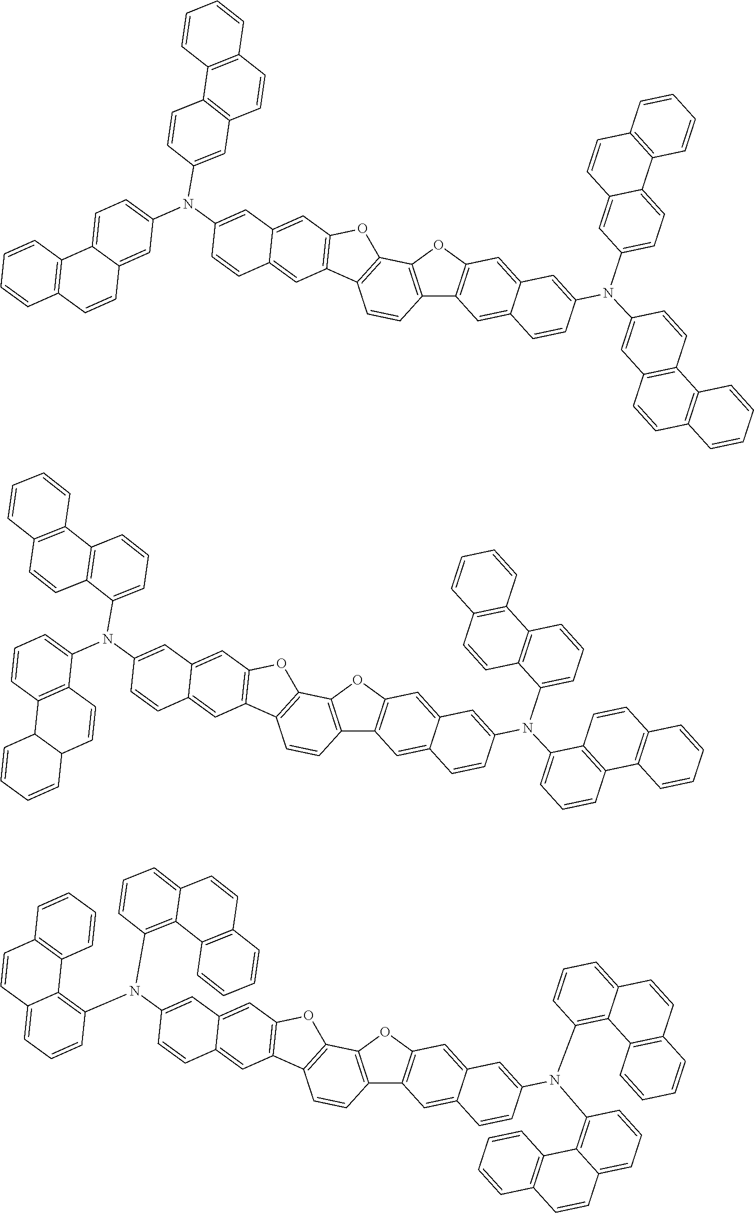 [SMILES: c1ccc2c(c1)ccc1cc(N(c3ccc4cc5c(cc4c3)oc3c5ccc4c5cc6ccc(N(c7ccc8c(ccc9ccccc98)c7)c7ccc8c(ccc9ccccc98)c7)cc6cc5oc43)c3ccc4c(ccc5ccccc54)c3)ccc12 ; C1=CC2C=Cc3c(cccc3N(c3ccc4cc5c(cc4c3)oc3c5ccc4c5cc6ccc(N(c7cccc8c7ccc7ccccc78)c7cccc8c7ccc7ccccc78)cc6cc5oc43)c3cccc4c3ccc3ccccc34)C2C=C1 ; c1ccc2c(c1)ccc1cccc(N(c3ccc4cc5c(cc4c3)oc3c5ccc4c5cc6ccc(N(c7cccc8ccc9ccccc9c78)c7cccc8ccc9ccccc9c78)cc6cc5oc43)c3cccc4ccc5ccccc5c34)c12]

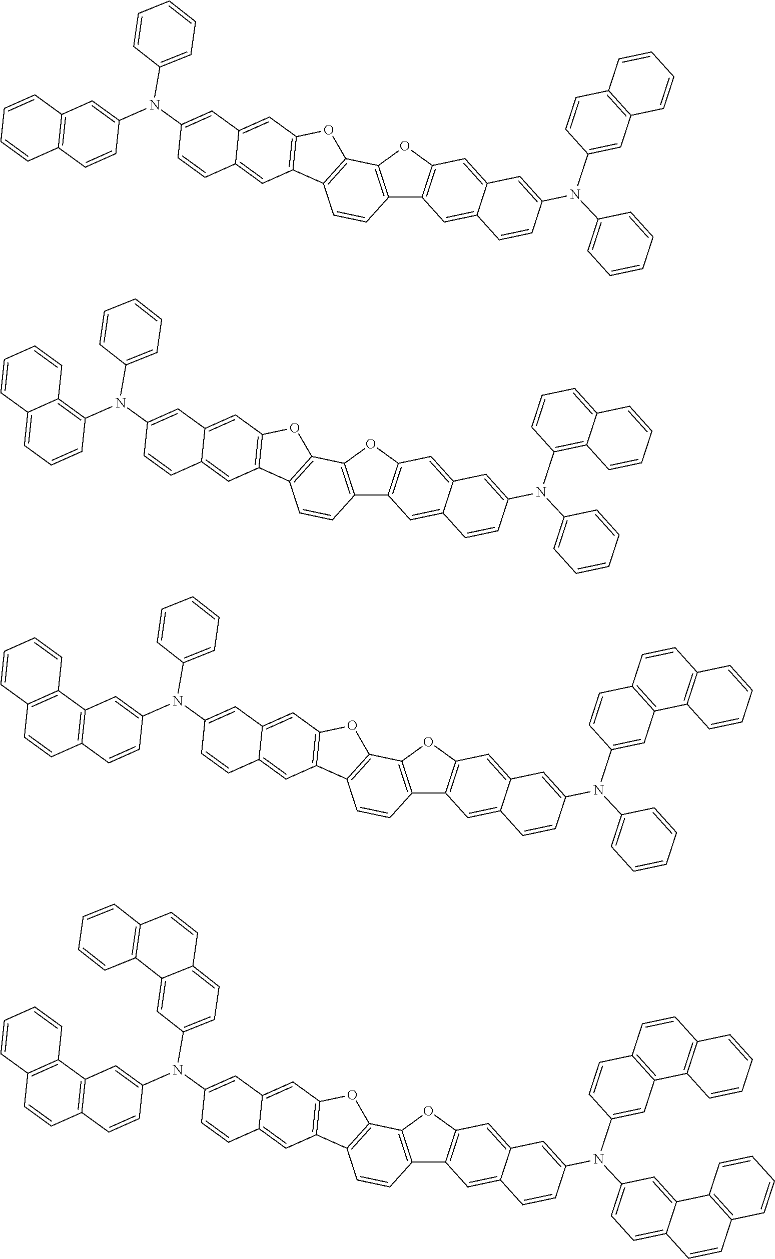 [SMILES: c1ccc(N(c2ccc3ccccc3c2)c2ccc3cc4c(cc3c2)oc2c4ccc3c4cc5ccc(N(c6ccccc6)c6ccc7ccccc7c6)cc5cc4oc32)cc1 ; c1ccc(N(c2ccc3cc4c(cc3c2)oc2c4ccc3c4cc5ccc(N(c6ccccc6)c6cccc7ccccc67)cc5cc4oc32)c2cccc3ccccc23)cc1 ; c1ccc(N(c2ccc3cc4c(cc3c2)oc2c4ccc3c4cc5ccc(N(c6ccccc6)c6ccc7ccc8ccccc8c7c6)cc5cc4oc32)c2ccc3ccc4ccccc4c3c2)cc1 ; c1ccc2c(c1)ccc1ccc(N(c3ccc4cc5c(cc4c3)oc3c5ccc4c5cc6ccc(N(c7ccc8ccc9ccccc9c8c7)c7ccc8ccc9ccccc9c8c7)cc6cc5oc43)c3ccc4ccc5ccccc5c4c3)cc12]

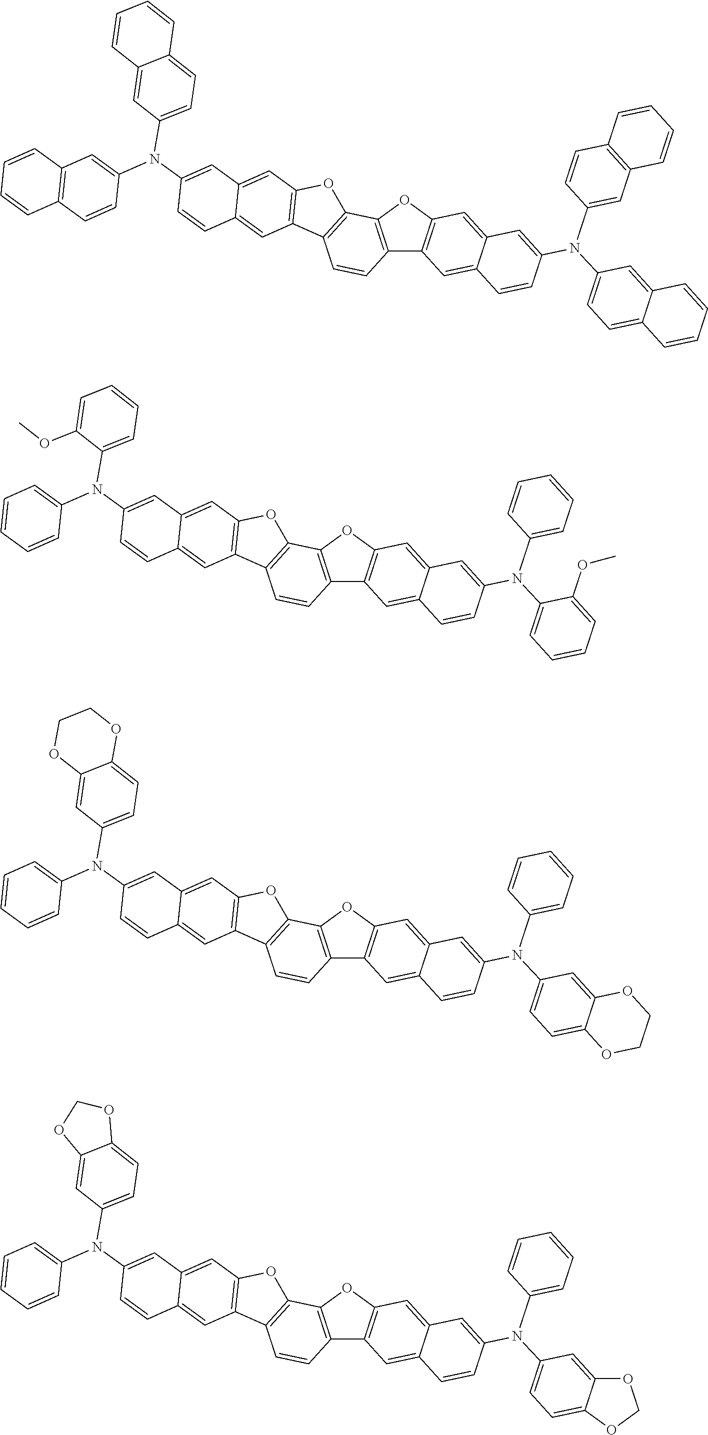 [SMILES: c1ccc2cc(N(c3ccc4ccccc4c3)c3ccc4cc5c(cc4c3)oc3c5ccc4c5cc6ccc(N(c7ccc8ccccc8c7)c7ccc8ccccc8c7)cc6cc5oc43)ccc2c1 ; COc1ccccc1N(c1ccccc1)c1ccc2cc3c(cc2c1)oc1c3ccc2c3cc4ccc(N(c5ccccc5)c5ccccc5OC)cc4cc3oc21 ; c1ccc(N(c2ccc3c(c2)OCCO3)c2ccc3cc4c(cc3c2)oc2c4ccc3c4cc5ccc(N(c6ccccc6)c6ccc7c(c6)OCCO7)cc5cc4oc32)cc1 ; c1ccc(N(c2ccc3c(c2)OCO3)c2ccc3cc4c(cc3c2)oc2c4ccc3c4cc5ccc(N(c6ccccc6)c6ccc7c(c6)OCO7)cc5cc4oc32)cc1]

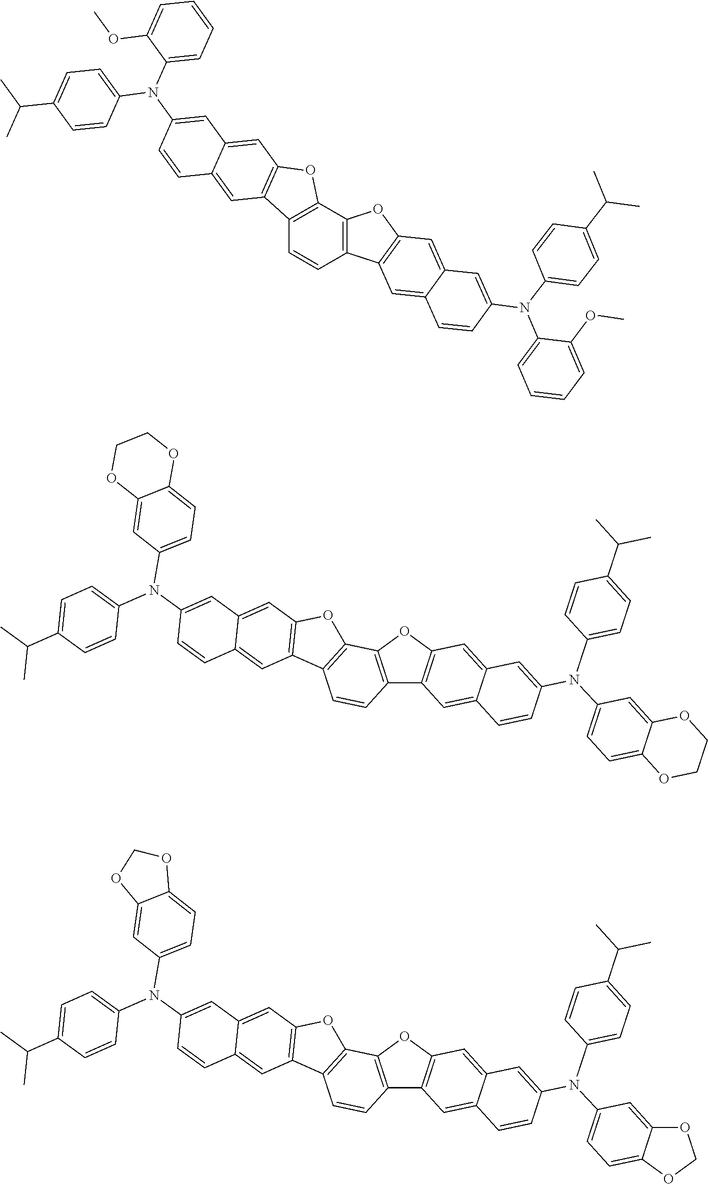

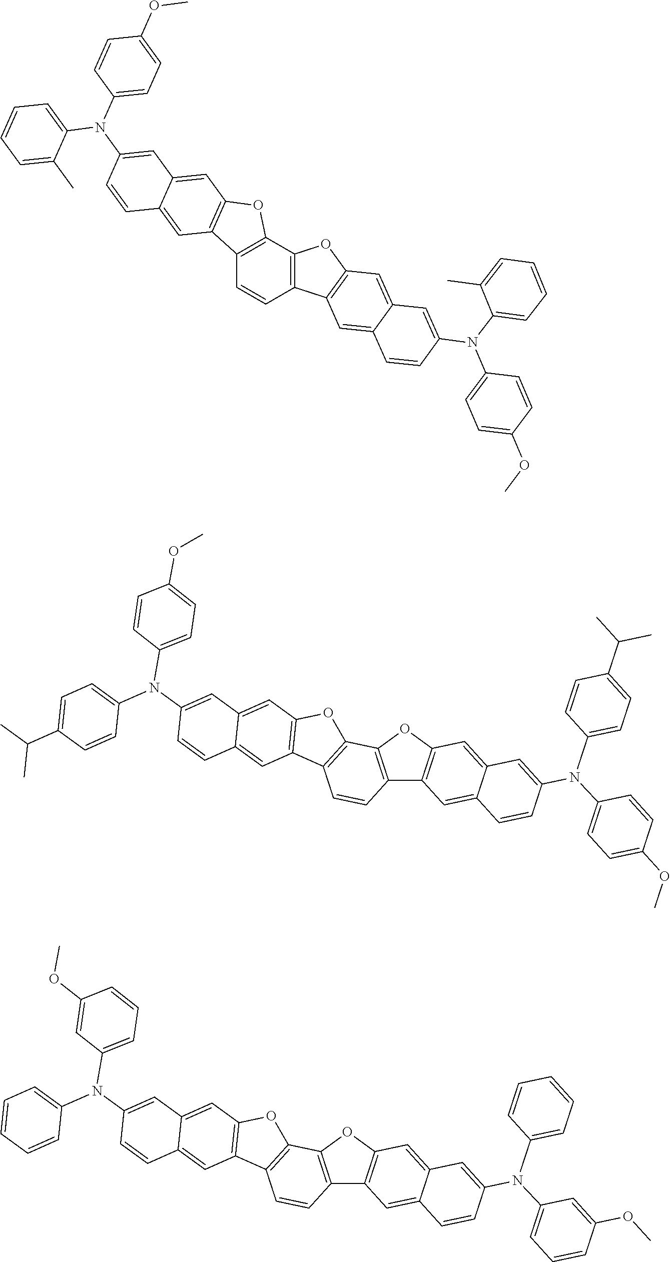 [SMILES: COc1ccc(N(c2ccc3cc4c(cc3c2)oc2c4ccc3c4cc5ccc(N(c6ccc(OC)cc6)c6ccccc6C)cc5cc4oc32)c2ccccc2C)cc1 ; COc1ccc(N(c2ccc(C(C)C)cc2)c2ccc3cc4c(cc3c2)oc2c4ccc3c4cc5ccc(N(c6ccc(OC)cc6)c6ccc(C(C)C)cc6)cc5cc4oc32)cc1 ; COc1cccc(N(c2ccccc2)c2ccc3cc4c(cc3c2)oc2c4ccc3c4cc5ccc(N(c6ccccc6)c6cccc(OC)c6)cc5cc4oc32)c1]

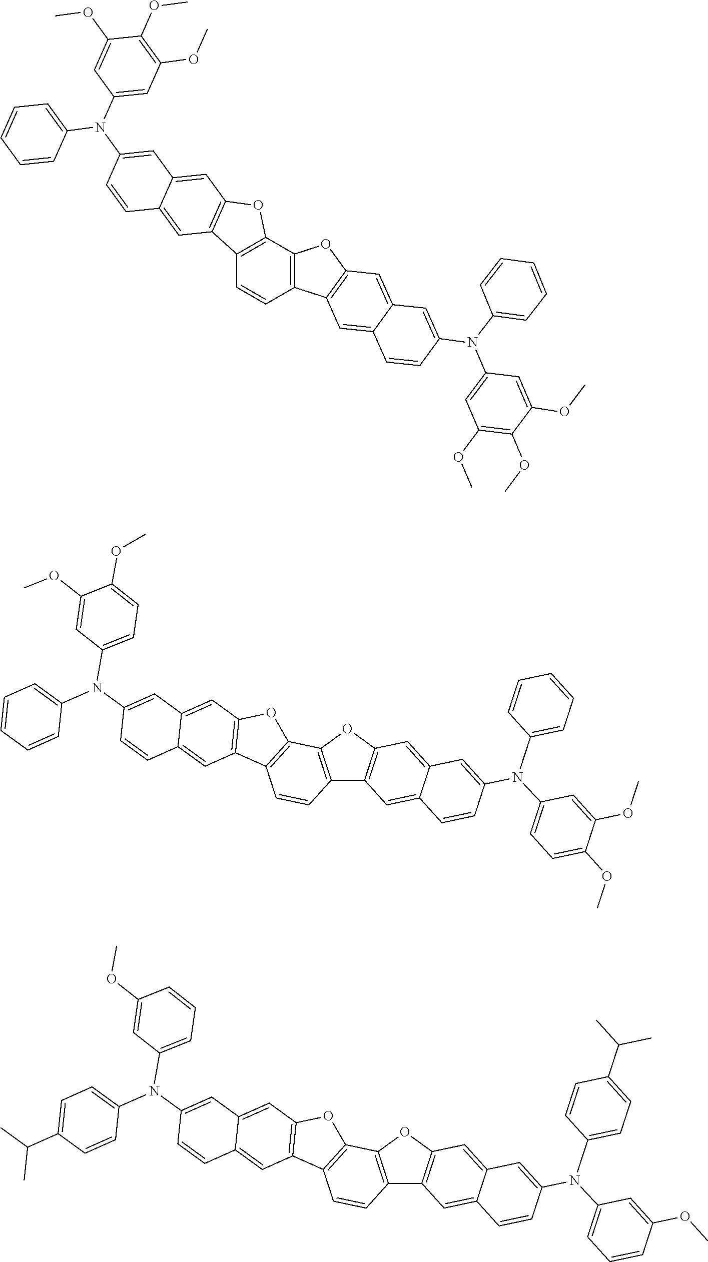

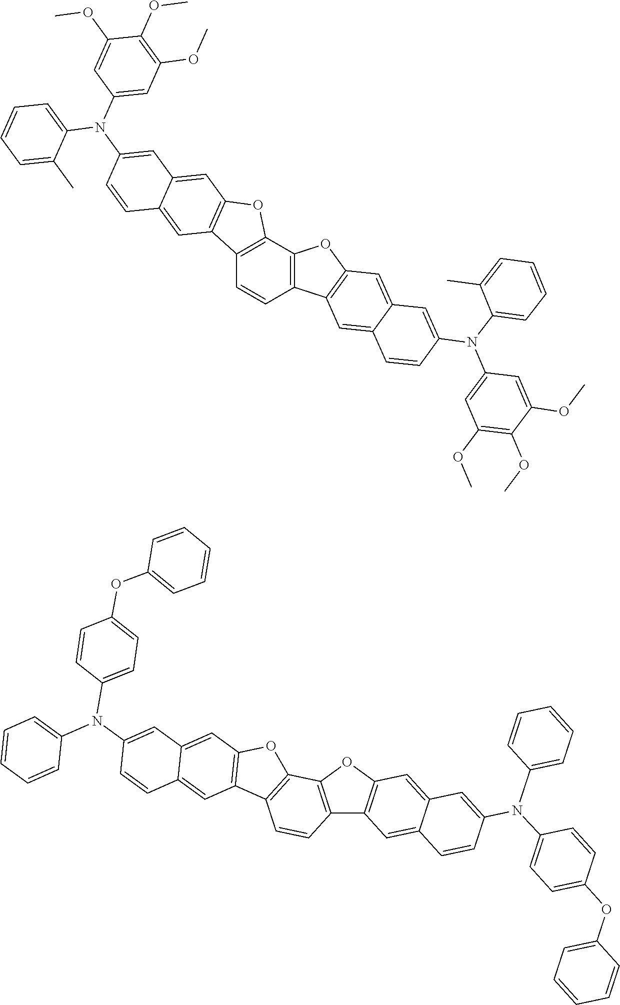 [SMILES: COc1cc(N(c2ccc3cc4c(cc3c2)oc2c4ccc3c4cc5ccc(N(c6cc(OC)c(OC)c(OC)c6)c6ccccc6C)cc5cc4oc32)c2ccccc2C)cc(OC)c1OC ; c1ccc(Oc2ccc(N(c3ccccc3)c3ccc4cc5c(cc4c3)oc3c5ccc4c5cc6ccc(N(c7ccccc7)c7ccc(Oc8ccccc8)cc7)cc6cc5oc43)cc2)cc1]

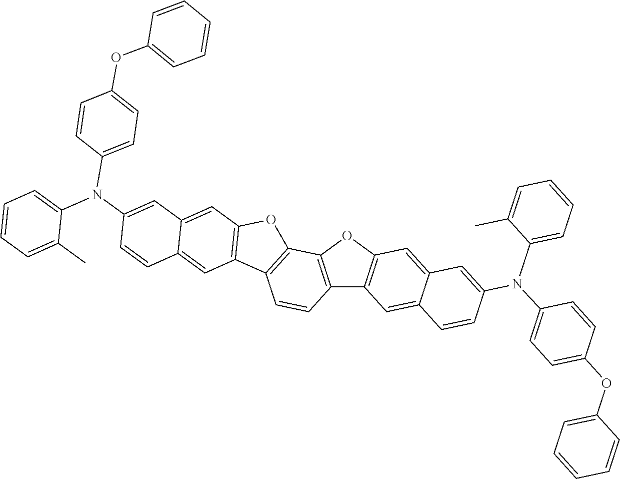 [SMILES: Cc1ccccc1N(c1ccc(Oc2ccccc2)cc1)c1ccc2cc3c(cc2c1)oc1c3ccc2c3cc4ccc(N(c5ccc(Oc6ccccc6)cc5)c5ccccc5C)cc4cc3oc21]

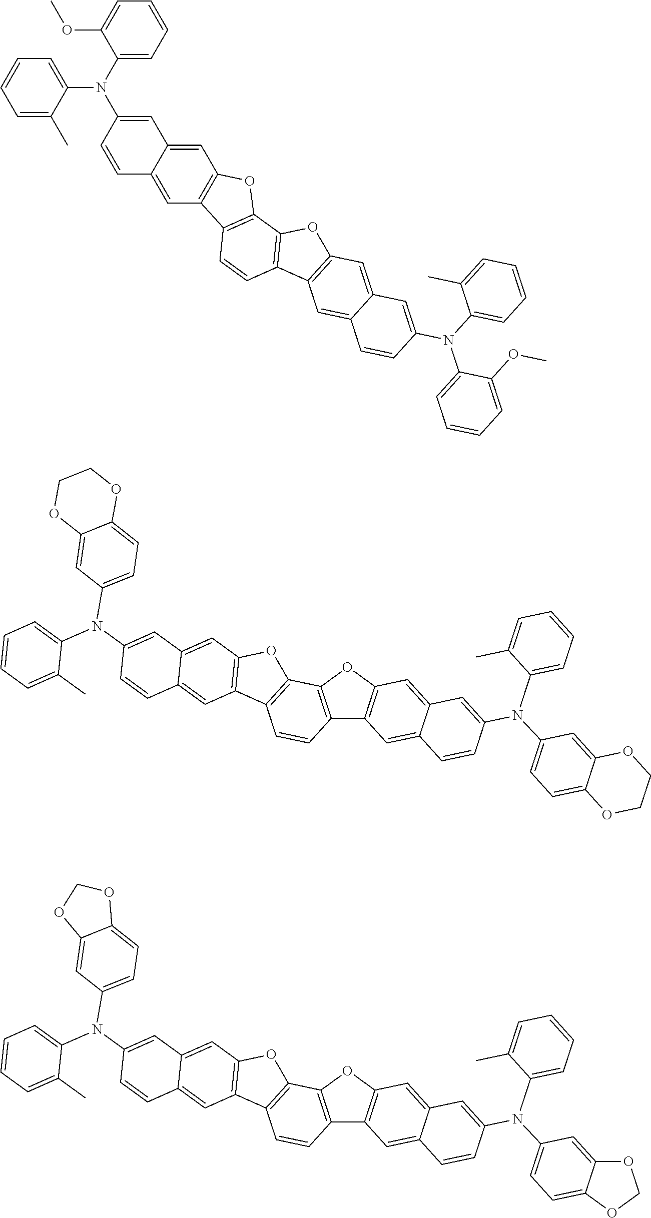 [SMILES: COc1ccccc1N(c1ccc2cc3c(cc2c1)oc1c3ccc2c3cc4ccc(N(c5ccccc5C)c5ccccc5OC)cc4cc3oc21)c1ccccc1C ; Cc1ccccc1N(c1ccc2c(c1)OCCO2)c1ccc2cc3c(cc2c1)oc1c3ccc2c3cc4ccc(N(c5ccc6c(c5)OCCO6)c5ccccc5C)cc4cc3oc21 ; Cc1ccccc1N(c1ccc2c(c1)OCO2)c1ccc2cc3c(cc2c1)oc1c3ccc2c3cc4ccc(N(c5ccc6c(c5)OCO6)c5ccccc5C)cc4cc3oc21]

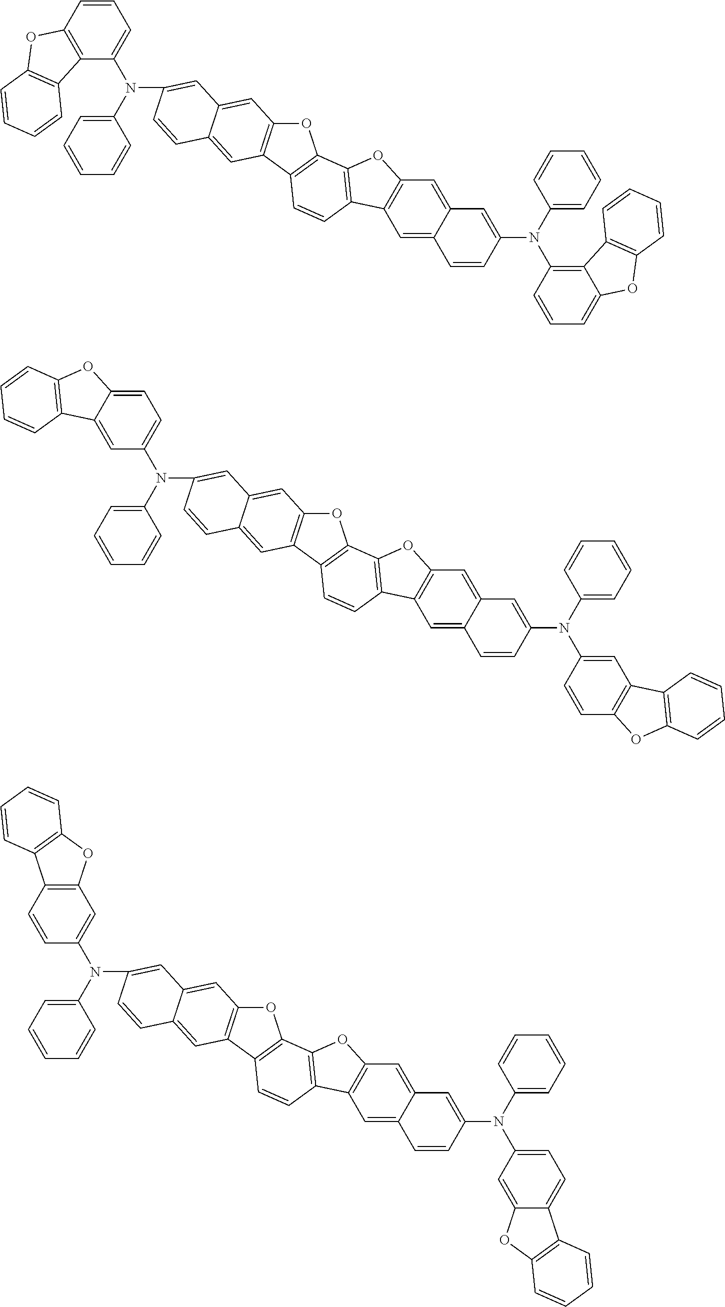 [SMILES: c1ccc(N(c2ccc3cc4c(cc3c2)oc2c4ccc3c4cc5ccc(N(c6ccccc6)c6cccc7oc8ccccc8c67)cc5cc4oc32)c2cccc3oc4ccccc4c23)cc1 ; c1ccc(N(c2ccc3cc4c(cc3c2)oc2c4ccc3c4cc5ccc(N(c6ccccc6)c6ccc7oc8ccccc8c7c6)cc5cc4oc32)c2ccc3oc4ccccc4c3c2)cc1 ; c1ccc(N(c2ccc3cc4c(cc3c2)oc2c4ccc3c4cc5ccc(N(c6ccccc6)c6ccc7c(c6)oc6ccccc67)cc5cc4oc32)c2ccc3c(c2)oc2ccccc23)cc1]

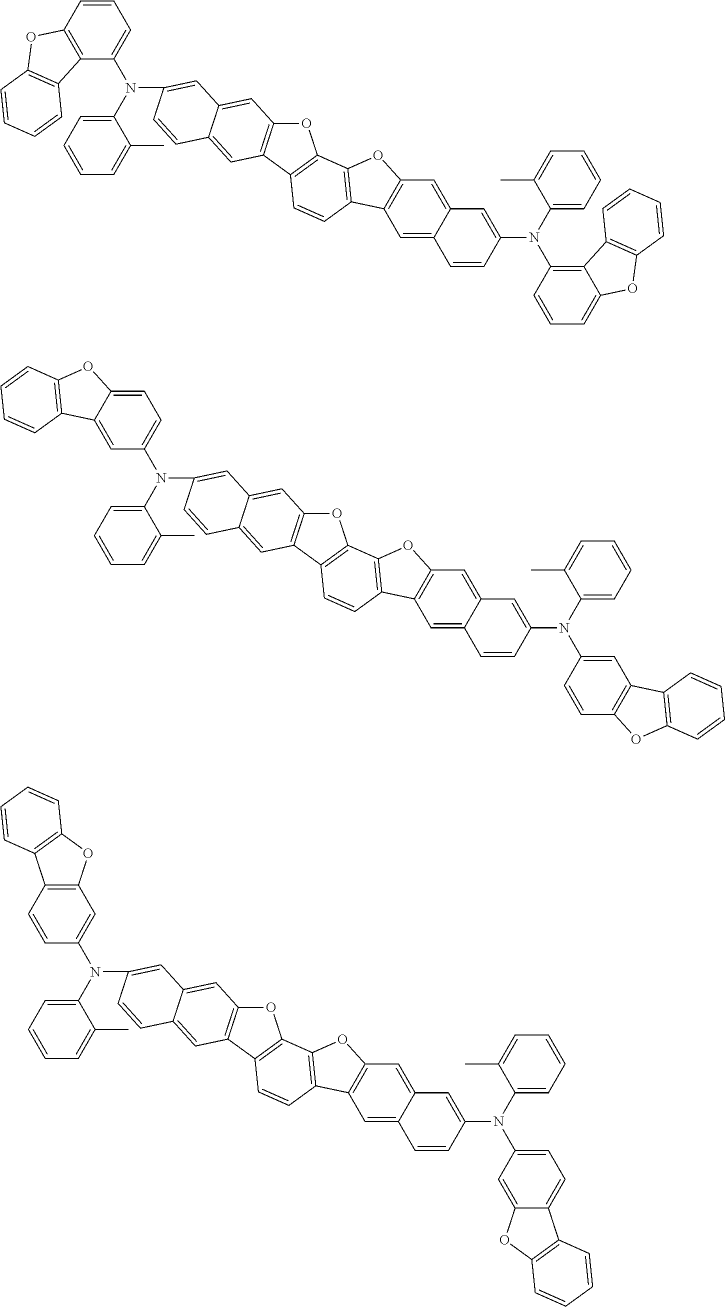 [SMILES: Cc1ccccc1N(c1ccc2cc3c(cc2c1)oc1c3ccc2c3cc4ccc(N(c5ccccc5C)c5cccc6oc7ccccc7c56)cc4cc3oc21)c1cccc2oc3ccccc3c12 ; Cc1ccccc1N(c1ccc2cc3c(cc2c1)oc1c3ccc2c3cc4ccc(N(c5ccc6oc7ccccc7c6c5)c5ccccc5C)cc4cc3oc21)c1ccc2oc3ccccc3c2c1 ; Cc1ccccc1N(c1ccc2cc3c(cc2c1)oc1c3ccc2c3cc4ccc(N(c5ccc6c(c5)oc5ccccc56)c5ccccc5C)cc4cc3oc21)c1ccc2c(c1)oc1ccccc12]

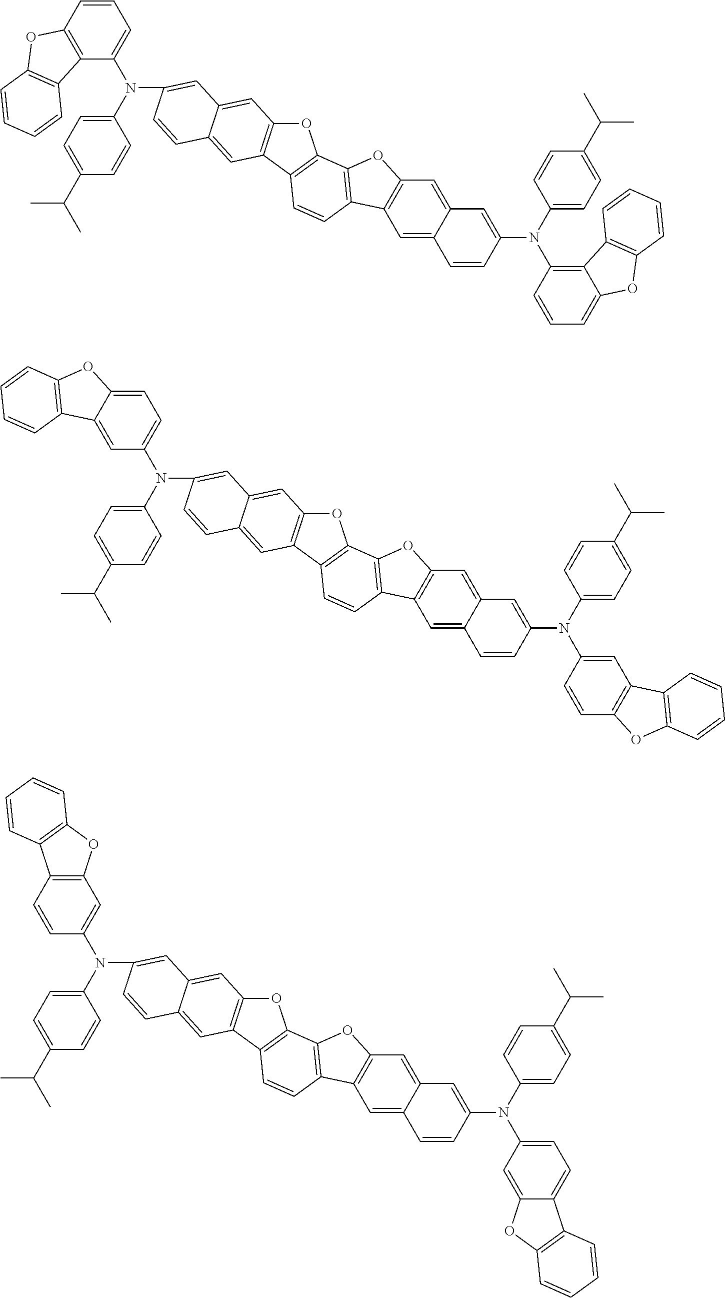 [SMILES: CC(C)c1ccc(N(c2ccc3cc4c(cc3c2)oc2c4ccc3c4cc5ccc(N(c6ccc(C(C)C)cc6)c6cccc7oc8ccccc8c67)cc5cc4oc32)c2cccc3oc4ccccc4c23)cc1 ; CC(C)c1ccc(N(c2ccc3cc4c(cc3c2)oc2c4ccc3c4cc5ccc(N(c6ccc(C(C)C)cc6)c6ccc7oc8ccccc8c7c6)cc5cc4oc32)c2ccc3oc4ccccc4c3c2)cc1 ; CC(C)c1ccc(N(c2ccc3cc4c(cc3c2)oc2c4ccc3c4cc5ccc(N(c6ccc(C(C)C)cc6)c6ccc7c(c6)oc6ccccc67)cc5cc4oc32)c2ccc3c(c2)oc2ccccc23)cc1]

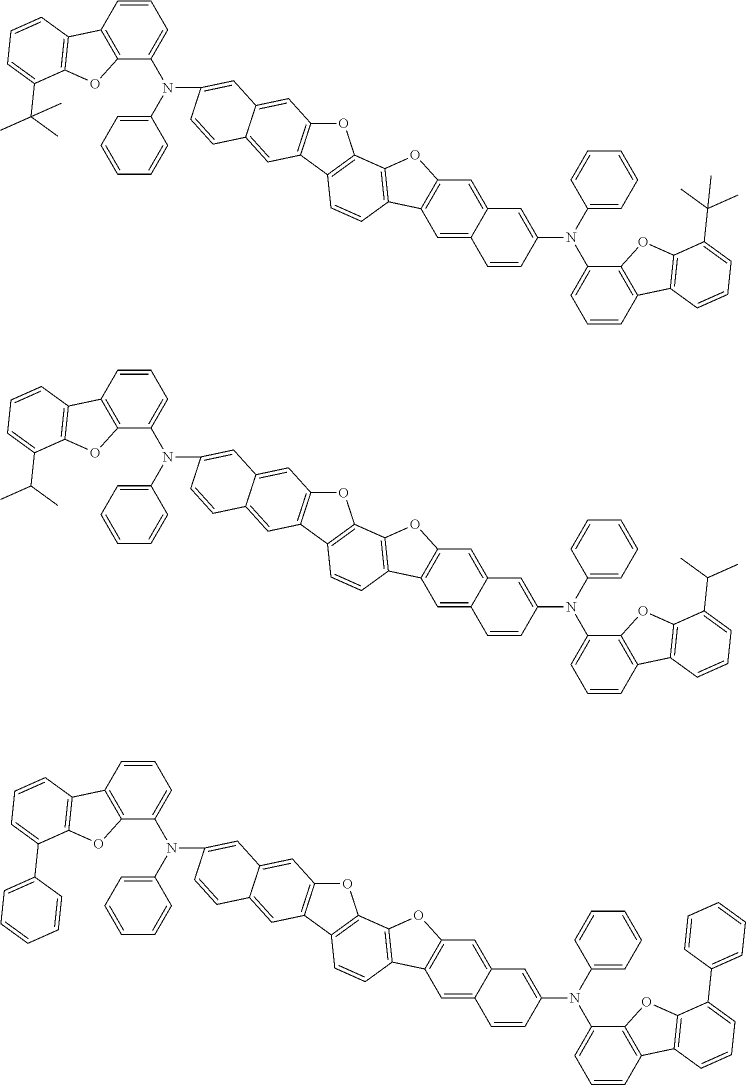

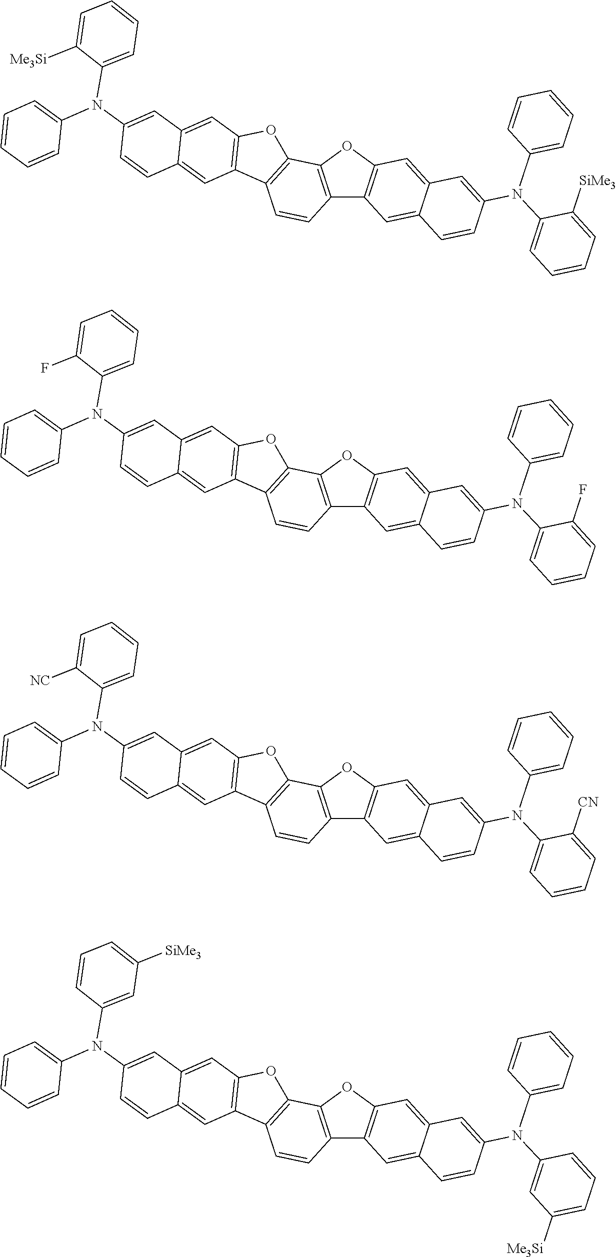 [SMILES: C[Si](C)(C)c1ccccc1N(c1ccccc1)c1ccc2cc3c(cc2c1)oc1c3ccc2c3cc4ccc(N(c5ccccc5)c5ccccc5[Si](C)(C)C)cc4cc3oc21 ; Fc1ccccc1N(c1ccccc1)c1ccc2cc3c(cc2c1)oc1c3ccc2c3cc4ccc(N(c5ccccc5)c5ccccc5F)cc4cc3oc21 ; N#Cc1ccccc1N(c1ccccc1)c1ccc2cc3c(cc2c1)oc1c3ccc2c3cc4ccc(N(c5ccccc5)c5ccccc5C#N)cc4cc3oc21 ; C[Si](C)(C)c1cccc(N(c2ccccc2)c2ccc3cc4c(cc3c2)oc2c4ccc3c4cc5ccc(N(c6ccccc6)c6cccc([Si](C)(C)C)c6)cc5cc4oc32)c1]

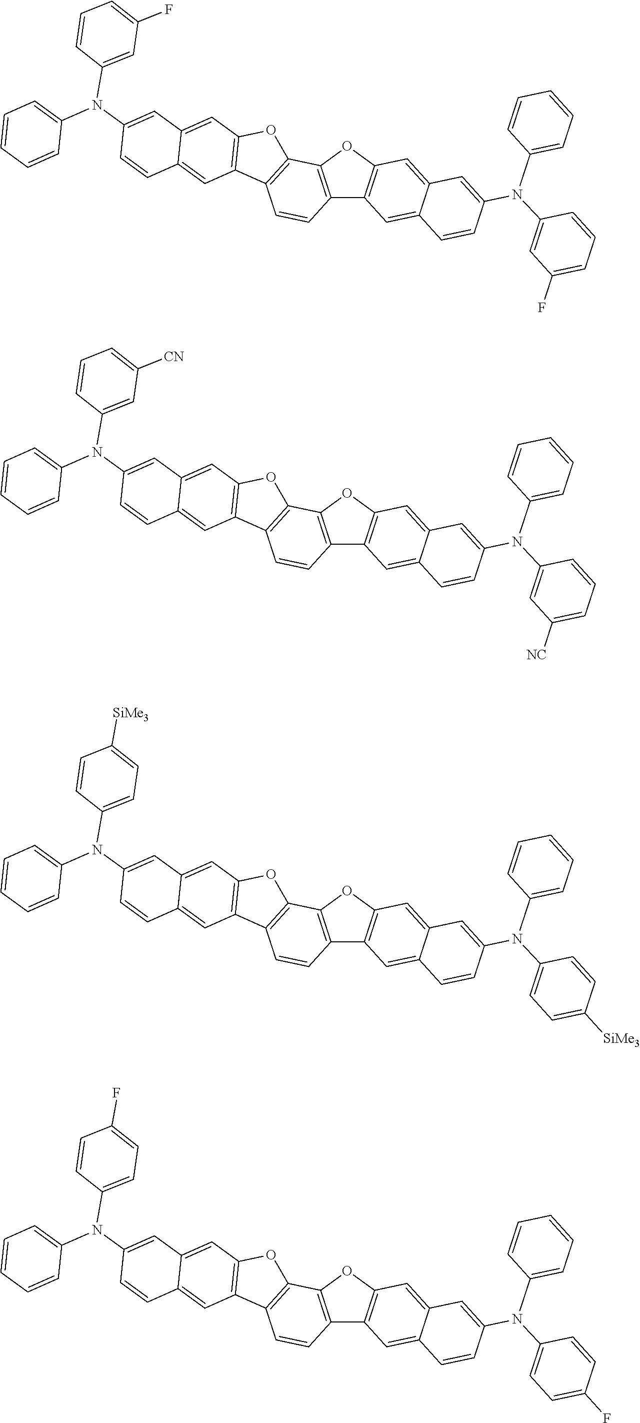 [SMILES: Fc1cccc(N(c2ccccc2)c2ccc3cc4c(cc3c2)oc2c4ccc3c4cc5ccc(N(c6ccccc6)c6cccc(F)c6)cc5cc4oc32)c1 ; N#Cc1cccc(N(c2ccccc2)c2ccc3cc4c(cc3c2)oc2c4ccc3c4cc5ccc(N(c6ccccc6)c6cccc(C#N)c6)cc5cc4oc32)c1 ; C[Si](C)(C)c1ccc(N(c2ccccc2)c2ccc3cc4c(cc3c2)oc2c4ccc3c4cc5ccc(N(c6ccccc6)c6ccc([Si](C)(C)C)cc6)cc5cc4oc32)cc1 ; Fc1ccc(N(c2ccccc2)c2ccc3cc4c(cc3c2)oc2c4ccc3c4cc5ccc(N(c6ccccc6)c6ccc(F)cc6)cc5cc4oc32)cc1]

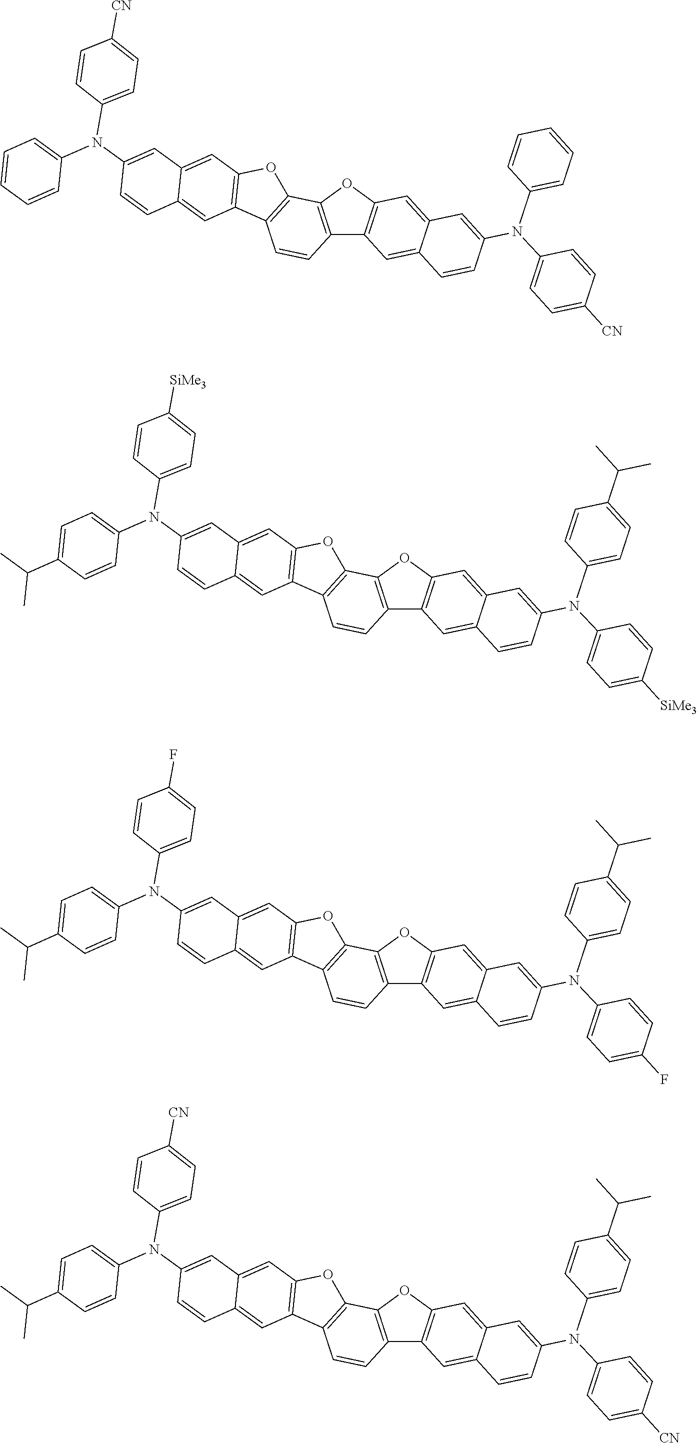 [SMILES: N#Cc1ccc(N(c2ccccc2)c2ccc3cc4c(cc3c2)oc2c4ccc3c4cc5ccc(N(c6ccccc6)c6ccc(C#N)cc6)cc5cc4oc32)cc1 ; CC(C)c1ccc(N(c2ccc([Si](C)(C)C)cc2)c2ccc3cc4c(cc3c2)oc2c4ccc3c4cc5ccc(N(c6ccc(C(C)C)cc6)c6ccc([Si](C)(C)C)cc6)cc5cc4oc32)cc1 ; CC(C)c1ccc(N(c2ccc(F)cc2)c2ccc3cc4c(cc3c2)oc2c4ccc3c4cc5ccc(N(c6ccc(F)cc6)c6ccc(C(C)C)cc6)cc5cc4oc32)cc1 ; CC(C)c1ccc(N(c2ccc(C#N)cc2)c2ccc3cc4c(cc3c2)oc2c4ccc3c4cc5ccc(N(c6ccc(C#N)cc6)c6ccc(C(C)C)cc6)cc5cc4oc32)cc1]

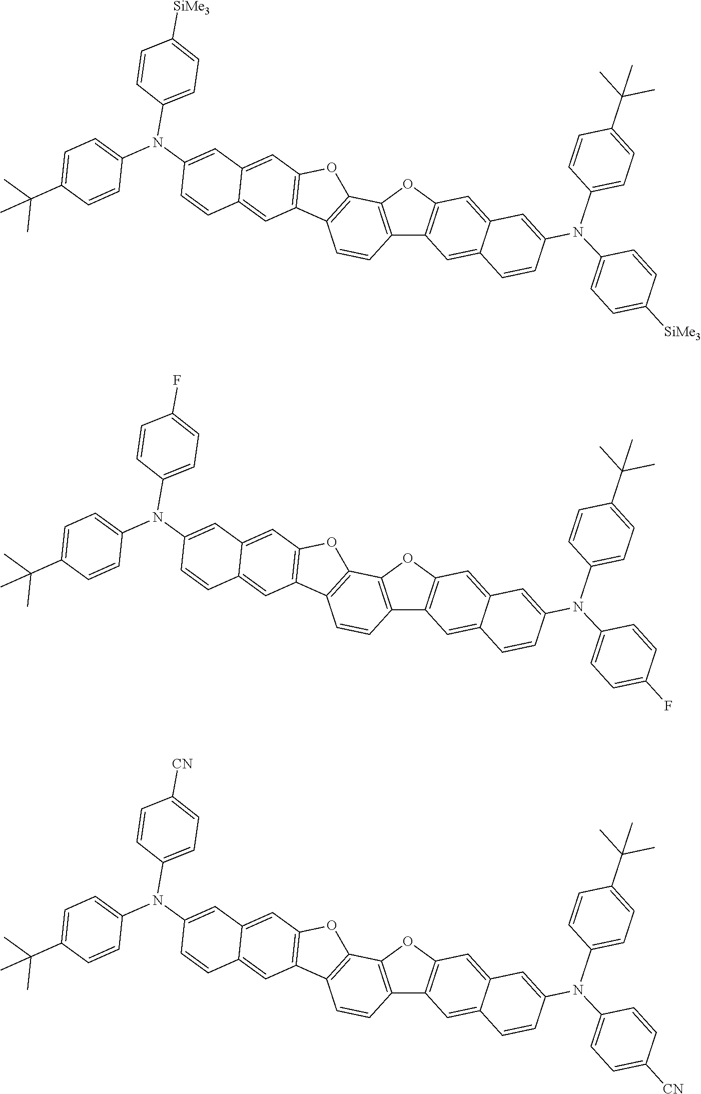 [SMILES: CC(C)(C)c1ccc(N(c2ccc([Si](C)(C)C)cc2)c2ccc3cc4c(cc3c2)oc2c4ccc3c4cc5ccc(N(c6ccc(C(C)(C)C)cc6)c6ccc([Si](C)(C)C)cc6)cc5cc4oc32)cc1 ; CC(C)(C)c1ccc(N(c2ccc(F)cc2)c2ccc3cc4c(cc3c2)oc2c4ccc3c4cc5ccc(N(c6ccc(F)cc6)c6ccc(C(C)(C)C)cc6)cc5cc4oc32)cc1 ; CC(C)(C)c1ccc(N(c2ccc(C#N)cc2)c2ccc3cc4c(cc3c2)oc2c4ccc3c4cc5ccc(N(c6ccc(C#N)cc6)c6ccc(C(C)(C)C)cc6)cc5cc4oc32)cc1]

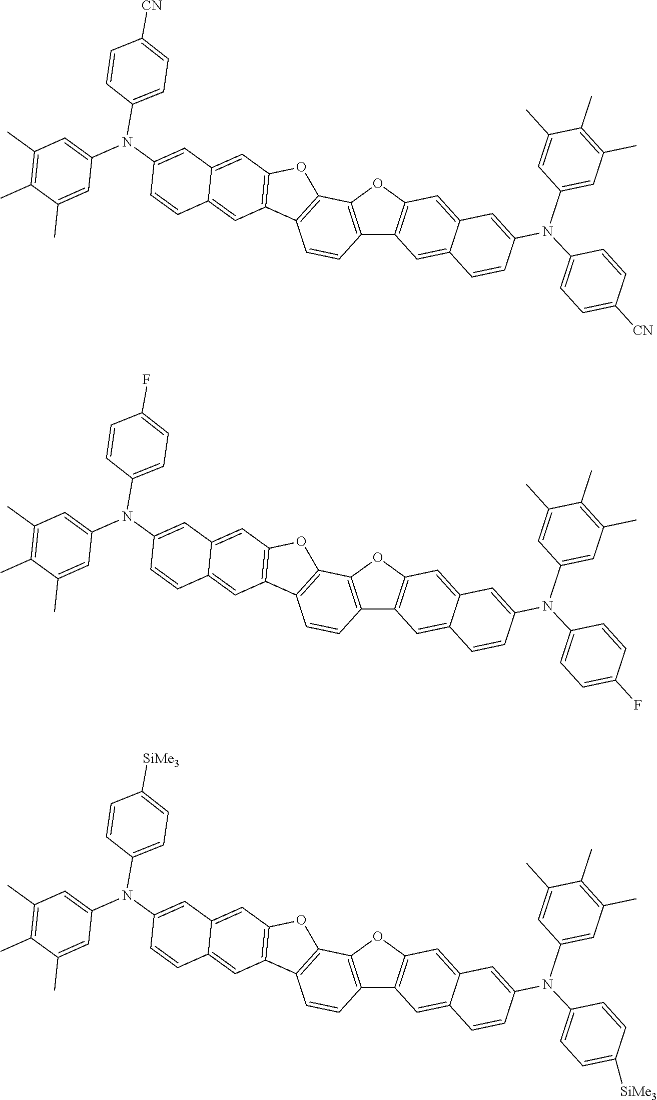

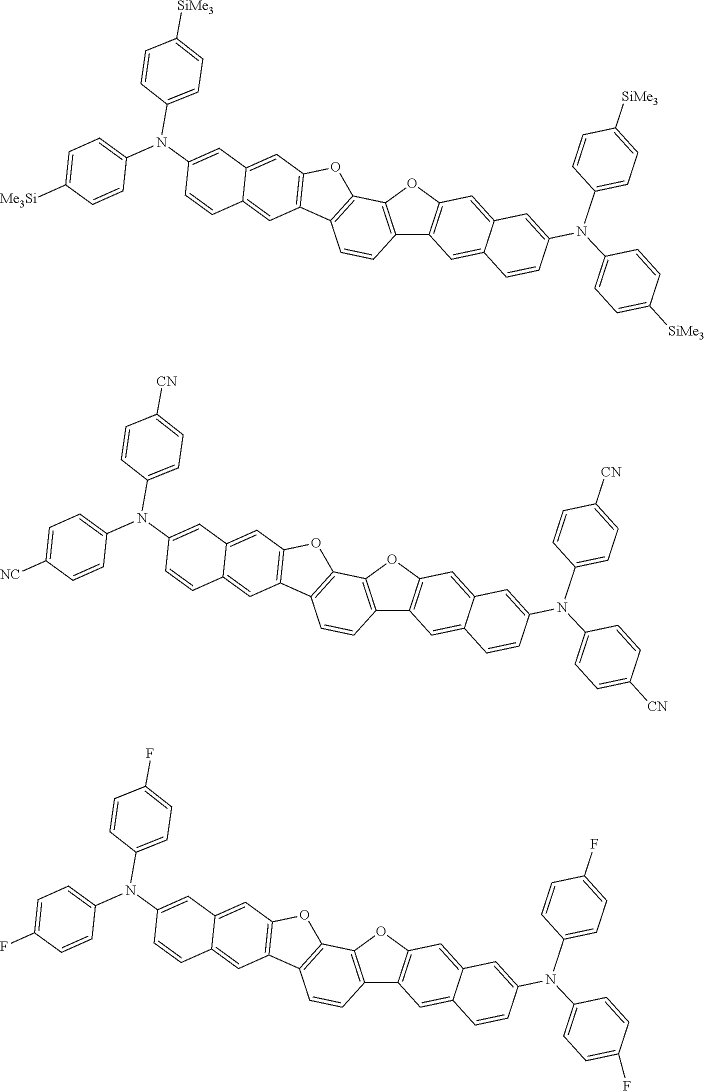 [SMILES: C[Si](C)(C)c1ccc(N(c2ccc([Si](C)(C)C)cc2)c2ccc3cc4c(cc3c2)oc2c4ccc3c4cc5ccc(N(c6ccc([Si](C)(C)C)cc6)c6ccc([Si](C)(C)C)cc6)cc5cc4oc32)cc1 ; N#Cc1ccc(N(c2ccc(C#N)cc2)c2ccc3cc4c(cc3c2)oc2c4ccc3c4cc5ccc(N(c6ccc(C#N)cc6)c6ccc(C#N)cc6)cc5cc4oc32)cc1 ; Fc1ccc(N(c2ccc(F)cc2)c2ccc3cc4c(cc3c2)oc2c4ccc3c4cc5ccc(N(c6ccc(F)cc6)c6ccc(F)cc6)cc5cc4oc32)cc1]

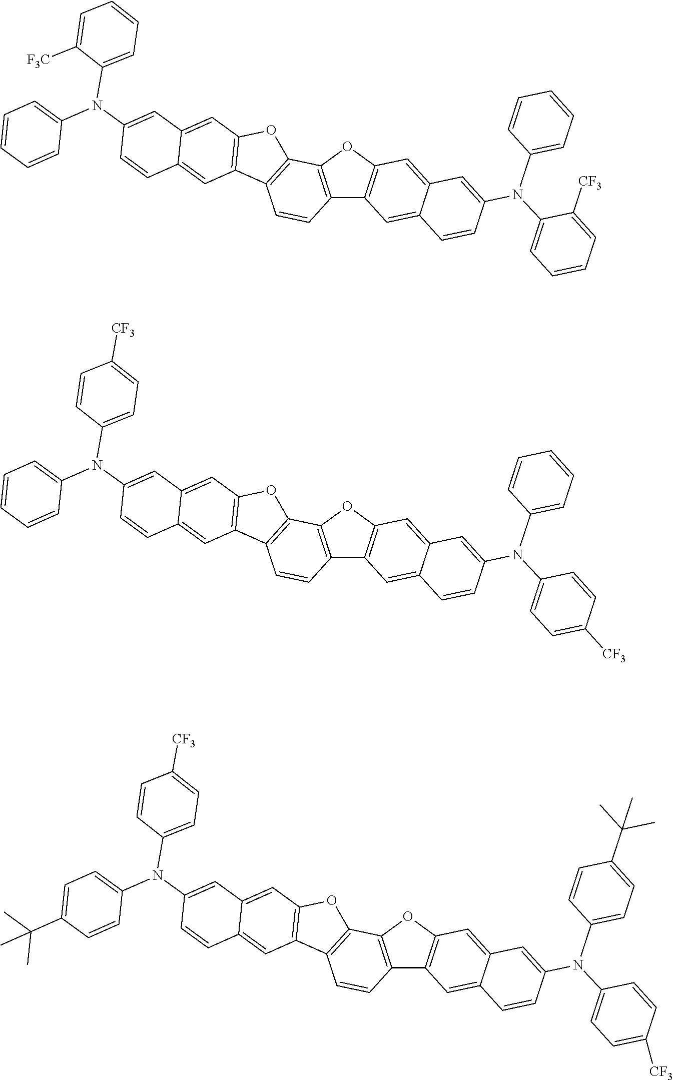 [SMILES: FC(F)(F)c1ccccc1N(c1ccccc1)c1ccc2cc3c(cc2c1)oc1c3ccc2c3cc4ccc(N(c5ccccc5)c5ccccc5C(F)(F)F)cc4cc3oc21 ; FC(F)(F)c1ccc(N(c2ccccc2)c2ccc3cc4c(cc3c2)oc2c4ccc3c4cc5ccc(N(c6ccccc6)c6ccc(C(F)(F)F)cc6)cc5cc4oc32)cc1 ; CC(C)(C)c1ccc(N(c2ccc(C(F)(F)F)cc2)c2ccc3cc4c(cc3c2)oc2c4ccc3c4cc5ccc(N(c6ccc(C(C)(C)C)cc6)c6ccc(C(F)(F)F)cc6)cc5cc4oc32)cc1]

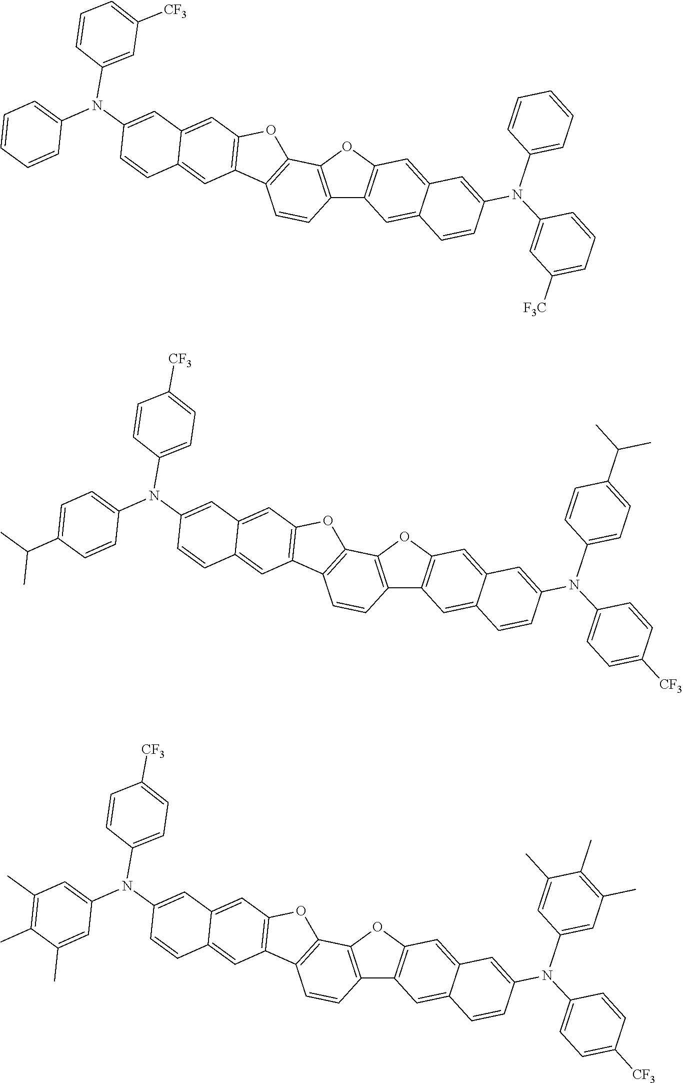 [SMILES: FC(F)(F)c1cccc(N(c2ccccc2)c2ccc3cc4c(cc3c2)oc2c4ccc3c4cc5ccc(N(c6ccccc6)c6cccc(C(F)(F)F)c6)cc5cc4oc32)c1 ; CC(C)c1ccc(N(c2ccc(C(F)(F)F)cc2)c2ccc3cc4c(cc3c2)oc2c4ccc3c4cc5ccc(N(c6ccc(C(C)C)cc6)c6ccc(C(F)(F)F)cc6)cc5cc4oc32)cc1 ; Cc1cc(N(c2ccc(C(F)(F)F)cc2)c2ccc3cc4c(cc3c2)oc2c4ccc3c4cc5ccc(N(c6ccc(C(F)(F)F)cc6)c6cc(C)c(C)c(C)c6)cc5cc4oc32)cc(C)c1C]

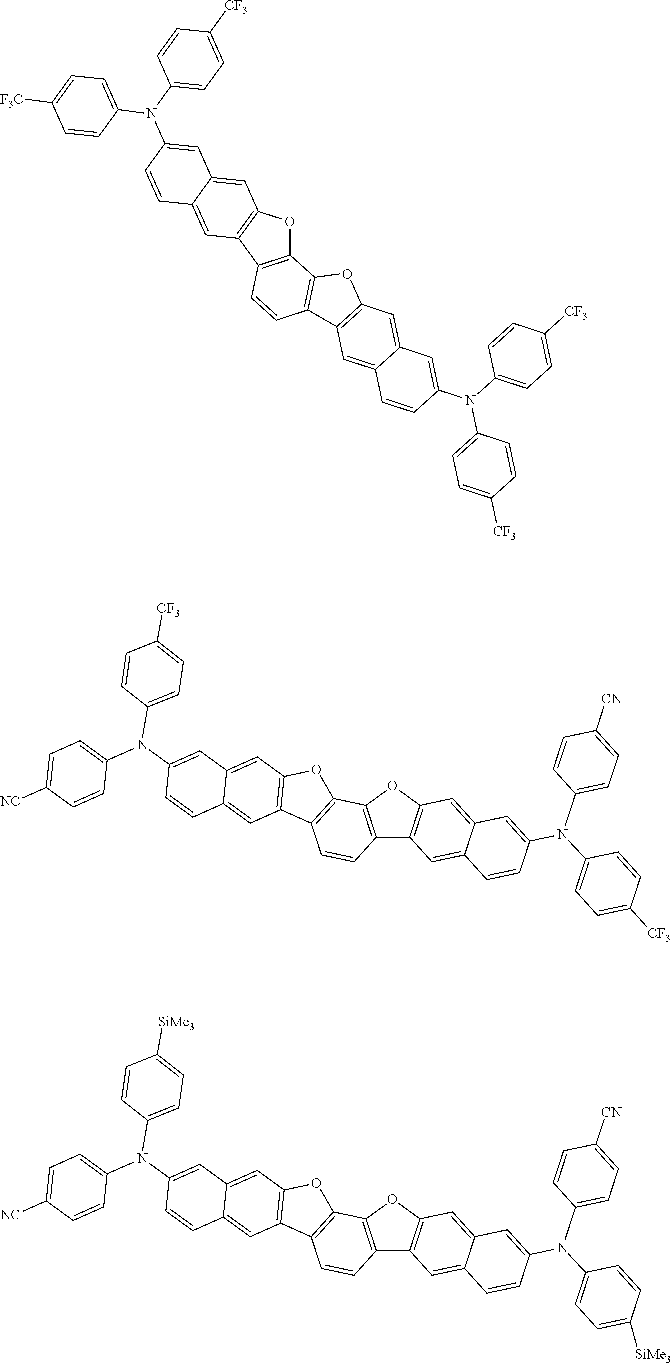 [SMILES: FC(F)(F)c1ccc(N(c2ccc(C(F)(F)F)cc2)c2ccc3cc4c(cc3c2)oc2c4ccc3c4cc5ccc(N(c6ccc(C(F)(F)F)cc6)c6ccc(C(F)(F)F)cc6)cc5cc4oc32)cc1 ; N#Cc1ccc(N(c2ccc(C(F)(F)F)cc2)c2ccc3cc4c(cc3c2)oc2c4ccc3c4cc5ccc(N(c6ccc(C#N)cc6)c6ccc(C(F)(F)F)cc6)cc5cc4oc32)cc1 ; C[Si](C)(C)c1ccc(N(c2ccc(C#N)cc2)c2ccc3cc4c(cc3c2)oc2c4ccc3c4cc5ccc(N(c6ccc(C#N)cc6)c6ccc([Si](C)(C)C)cc6)cc5cc4oc32)cc1]

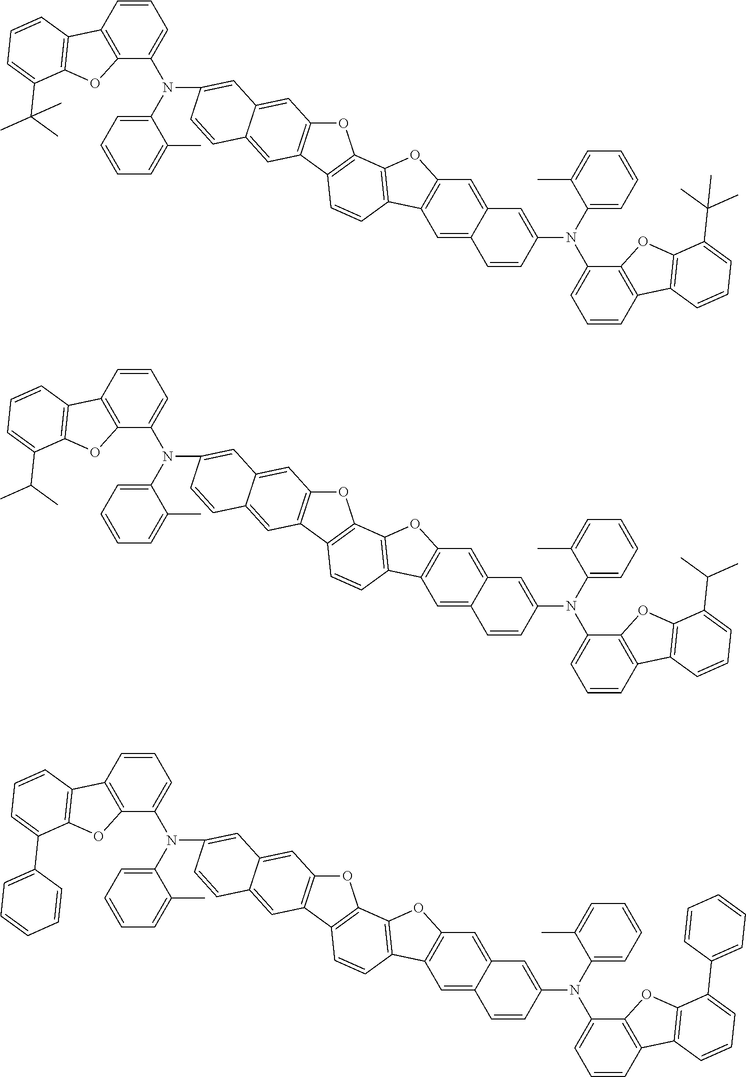

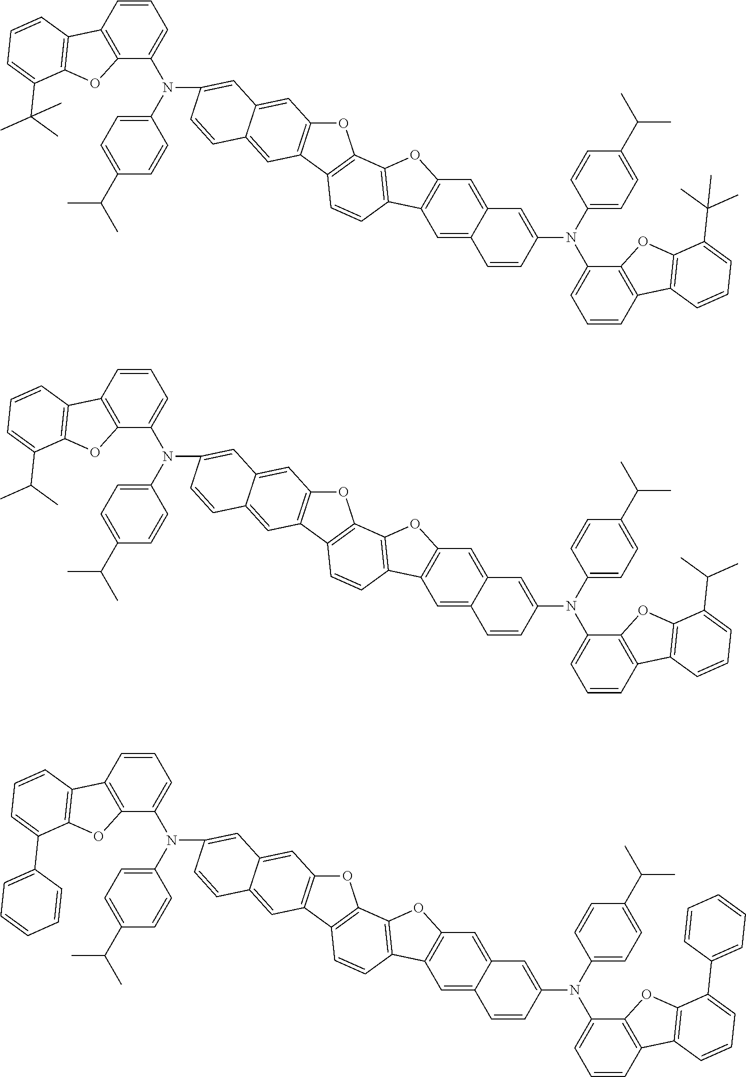

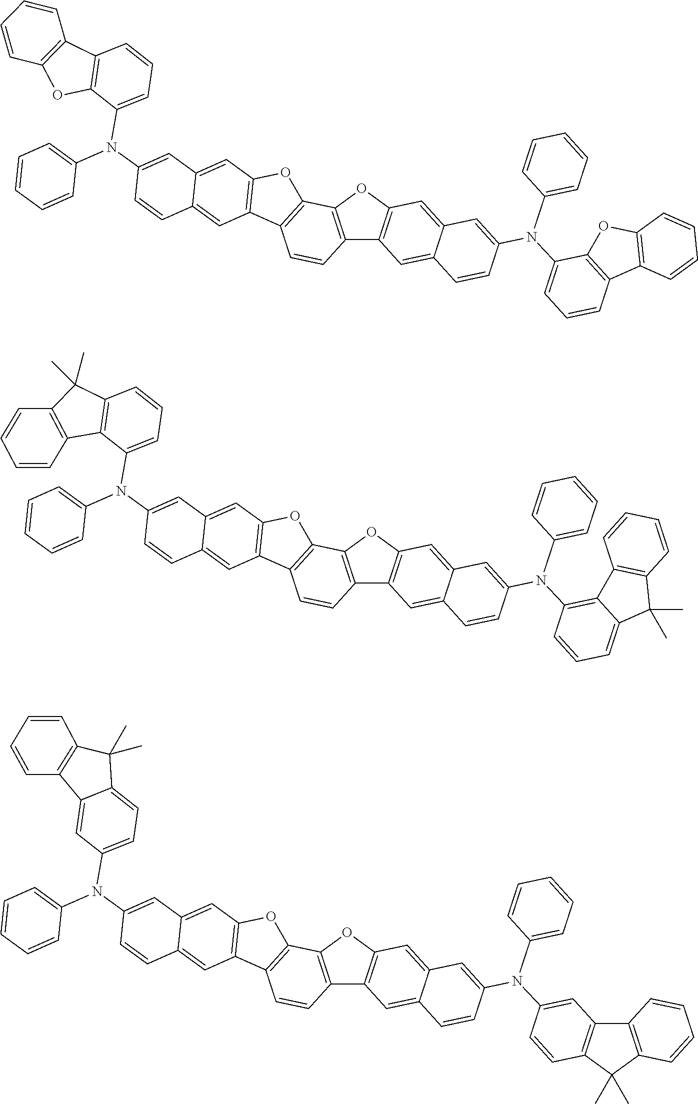 [SMILES: c1ccc(N(c2ccc3cc4c(cc3c2)oc2c4ccc3c4cc5ccc(N(c6ccccc6)c6cccc7c6oc6ccccc67)cc5cc4oc32)c2cccc3c2oc2ccccc23)cc1 ; CC1(C)c2ccccc2-c2c(N(c3ccccc3)c3ccc4cc5c(cc4c3)oc3c5ccc4c5cc6ccc(N(c7ccccc7)c7cccc8c7-c7ccccc7C8(C)C)cc6cc5oc43)cccc21 ; CC1(C)c2ccccc2-c2cc(N(c3ccccc3)c3ccc4cc5c(cc4c3)oc3c5ccc4c5cc6ccc(N(c7ccccc7)c7ccc8c(c7)-c7ccccc7C8(C)C)cc6cc5oc43)ccc21]

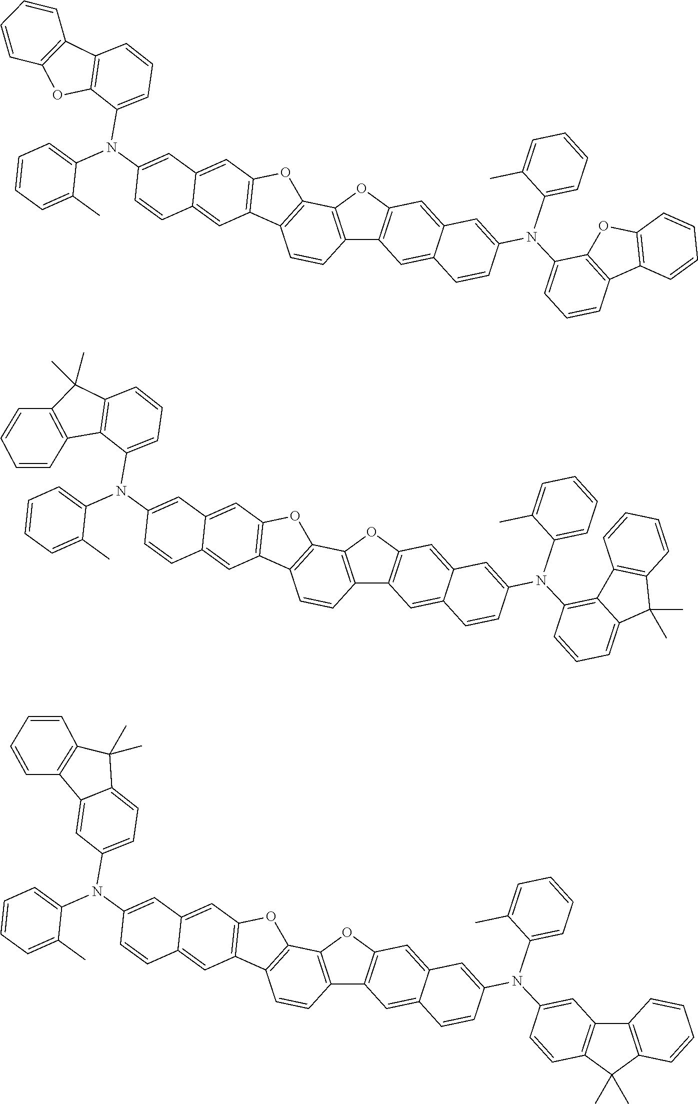 [SMILES: Cc1ccccc1N(c1ccc2cc3c(cc2c1)oc1c3ccc2c3cc4ccc(N(c5ccccc5C)c5cccc6c5oc5ccccc56)cc4cc3oc21)c1cccc2c1oc1ccccc12 ; Cc1ccccc1N(c1ccc2cc3c(cc2c1)oc1c3ccc2c3cc4ccc(N(c5ccccc5C)c5cccc6c5-c5ccccc5C6(C)C)cc4cc3oc21)c1cccc2c1-c1ccccc1C2(C)C ; Cc1ccccc1N(c1ccc2c(c1)-c1ccccc1C2(C)C)c1ccc2cc3c(cc2c1)oc1c3ccc2c3cc4ccc(N(c5ccc6c(c5)-c5ccccc5C6(C)C)c5ccccc5C)cc4cc3oc21]

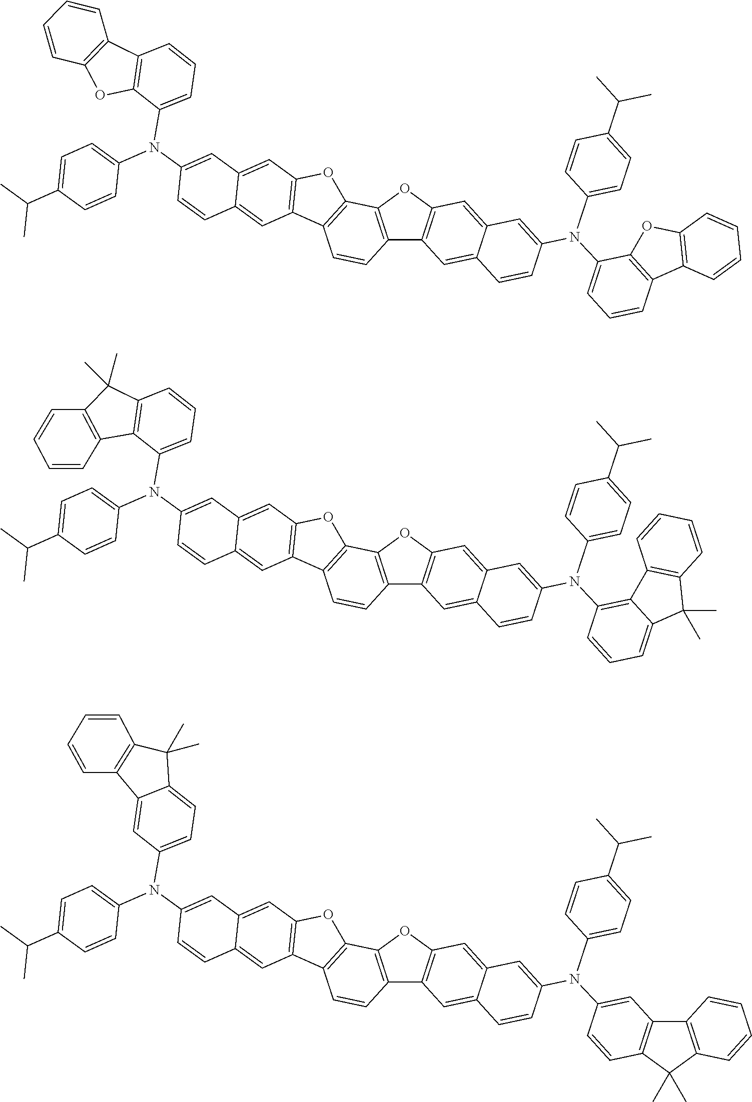

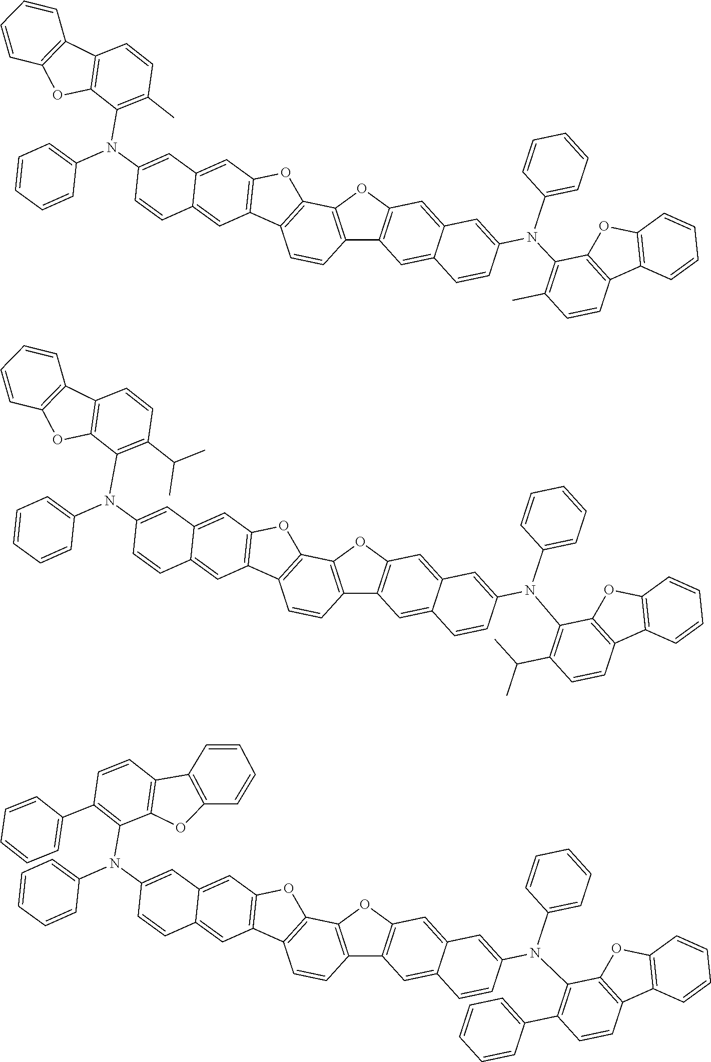 [SMILES: Cc1ccc2c(oc3ccccc32)c1N(c1ccccc1)c1ccc2cc3c(cc2c1)oc1c3ccc2c3cc4ccc(N(c5ccccc5)c5c(C)ccc6c5oc5ccccc56)cc4cc3oc21 ; CC(C)c1ccc2c(oc3ccccc32)c1N(c1ccccc1)c1ccc2cc3c(cc2c1)oc1c3ccc2c3cc4ccc(N(c5ccccc5)c5c(C(C)C)ccc6c5oc5ccccc56)cc4cc3oc21 ; c1ccc(-c2ccc3c(oc4ccccc43)c2N(c2ccccc2)c2ccc3cc4c(cc3c2)oc2c4ccc3c4cc5ccc(N(c6ccccc6)c6c(-c7ccccc7)ccc7c6oc6ccccc67)cc5cc4oc32)cc1]

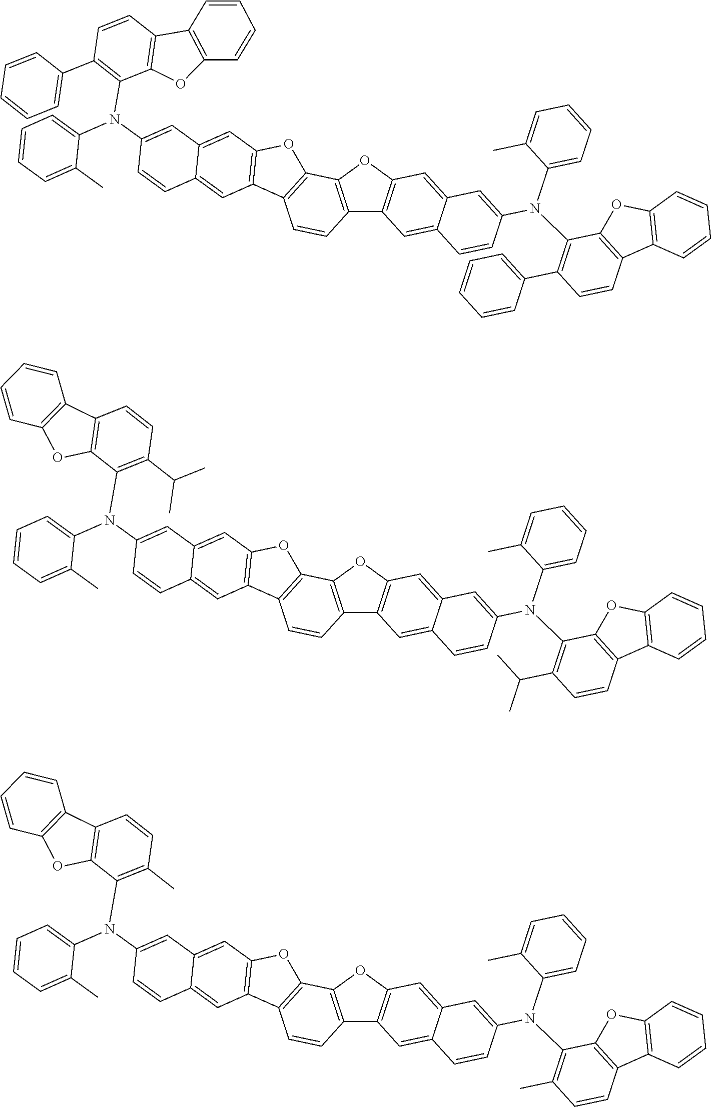 [SMILES: Cc1ccccc1N(c1ccc2cc3c(cc2c1)oc1c3ccc2c3cc4ccc(N(c5ccccc5C)c5c(-c6ccccc6)ccc6c5oc5ccccc56)cc4cc3oc21)c1c(-c2ccccc2)ccc2c1oc1ccccc12 ; Cc1ccccc1N(c1ccc2cc3c(cc2c1)oc1c3ccc2c3cc4ccc(N(c5ccccc5C)c5c(C(C)C)ccc6c5oc5ccccc56)cc4cc3oc21)c1c(C(C)C)ccc2c1oc1ccccc12 ; Cc1ccccc1N(c1ccc2cc3c(cc2c1)oc1c3ccc2c3cc4ccc(N(c5ccccc5C)c5c(C)ccc6c5oc5ccccc56)cc4cc3oc21)c1c(C)ccc2c1oc1ccccc12]

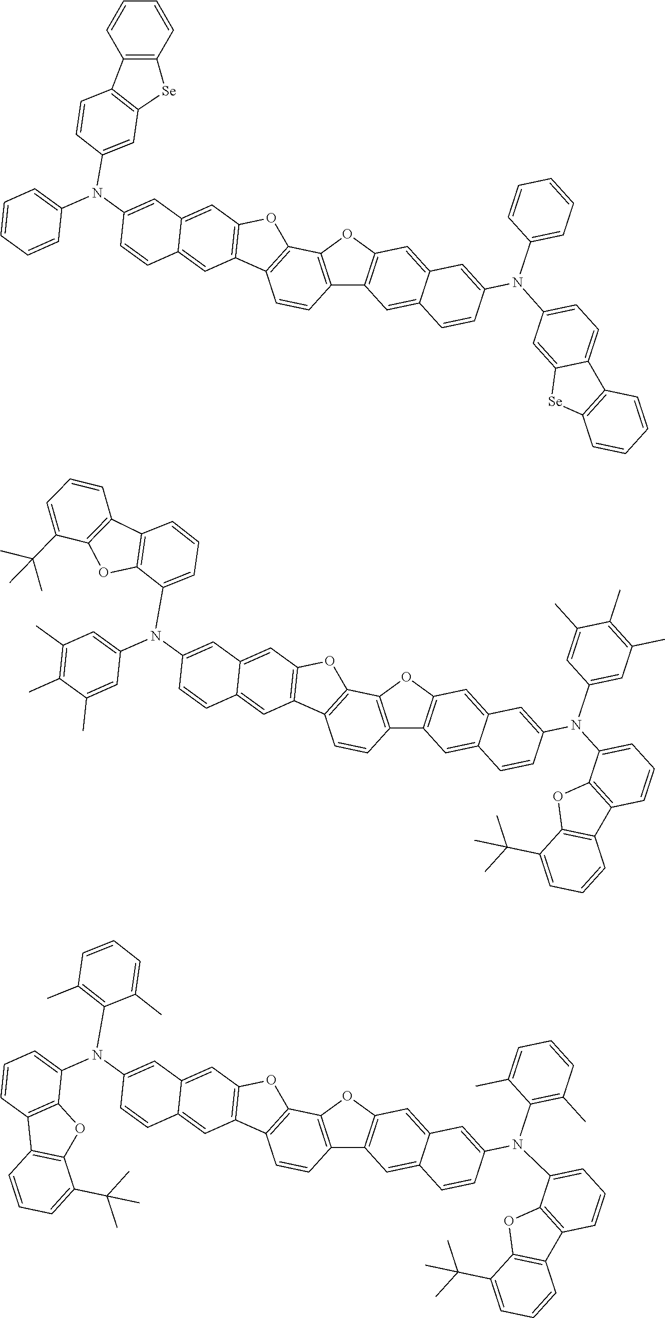 [SMILES: c1ccc(N(c2ccc3cc4c(cc3c2)oc2c4ccc3c4cc5ccc(N(c6ccccc6)c6ccc7c(c6)[se]c6ccccc67)cc5cc4oc32)c2ccc3c(c2)[se]c2ccccc23)cc1 ; Cc1cc(N(c2ccc3cc4c(cc3c2)oc2c4ccc3c4cc5ccc(N(c6cc(C)c(C)c(C)c6)c6cccc7c6oc6c(C(C)(C)C)cccc67)cc5cc4oc32)c2cccc3c2oc2c(C(C)(C)C)cccc23)cc(C)c1C ; Cc1cccc(C)c1N(c1ccc2cc3c(cc2c1)oc1c3ccc2c3cc4ccc(N(c5c(C)cccc5C)c5cccc6c5oc5c(C(C)(C)C)cccc56)cc4cc3oc21)c1cccc2c1oc1c(C(C)(C)C)cccc12]

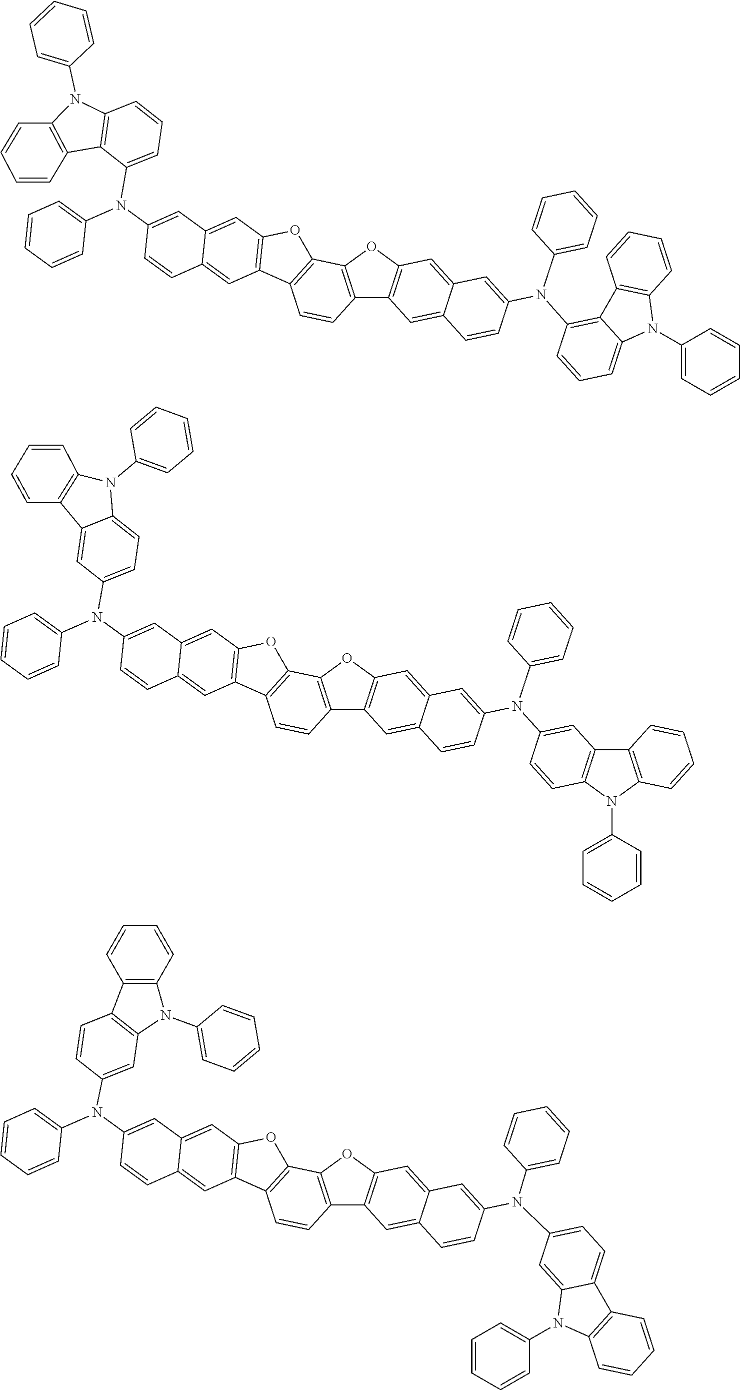 [SMILES: c1ccc(N(c2ccc3cc4c(cc3c2)oc2c4ccc3c4cc5ccc(N(c6ccccc6)c6cccc7c6c6ccccc6n7-c6ccccc6)cc5cc4oc32)c2cccc3c2c2ccccc2n3-c2ccccc2)cc1 ; c1ccc(N(c2ccc3cc4c(cc3c2)oc2c4ccc3c4cc5ccc(N(c6ccccc6)c6ccc7c(c6)c6ccccc6n7-c6ccccc6)cc5cc4oc32)c2ccc3c(c2)c2ccccc2n3-c2ccccc2)cc1 ; c1ccc(N(c2ccc3cc4c(cc3c2)oc2c4ccc3c4cc5ccc(N(c6ccccc6)c6ccc7c8ccccc8n(-c8ccccc8)c7c6)cc5cc4oc32)c2ccc3c4ccccc4n(-c4ccccc4)c3c2)cc1]

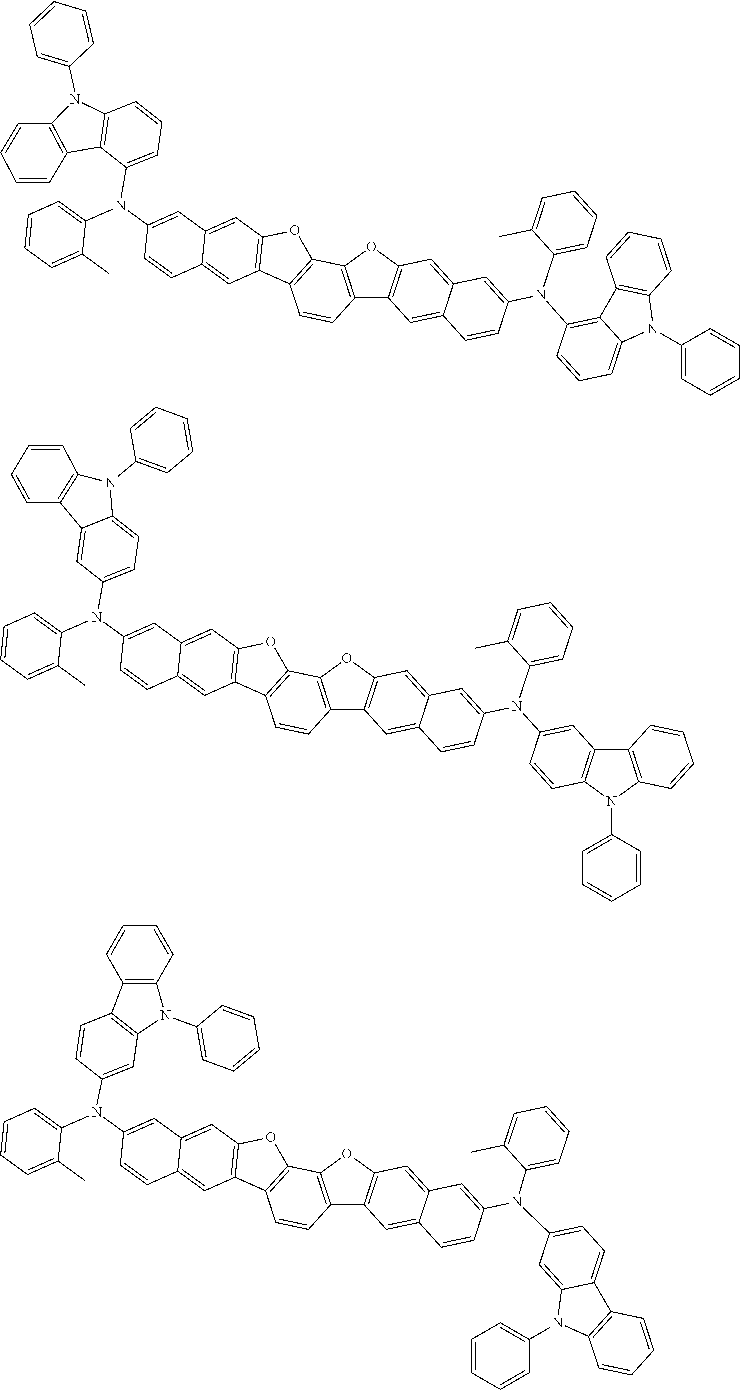 [SMILES: Cc1ccccc1N(c1ccc2cc3c(cc2c1)oc1c3ccc2c3cc4ccc(N(c5ccccc5C)c5cccc6c5c5ccccc5n6-c5ccccc5)cc4cc3oc21)c1cccc2c1c1ccccc1n2-c1ccccc1 ; Cc1ccccc1N(c1ccc2cc3c(cc2c1)oc1c3ccc2c3cc4ccc(N(c5ccc6c(c5)c5ccccc5n6-c5ccccc5)c5ccccc5C)cc4cc3oc21)c1ccc2c(c1)c1ccccc1n2-c1ccccc1 ; Cc1ccccc1N(c1ccc2cc3c(cc2c1)oc1c3ccc2c3cc4ccc(N(c5ccc6c7ccccc7n(-c7ccccc7)c6c5)c5ccccc5C)cc4cc3oc21)c1ccc2c3ccccc3n(-c3ccccc3)c2c1]

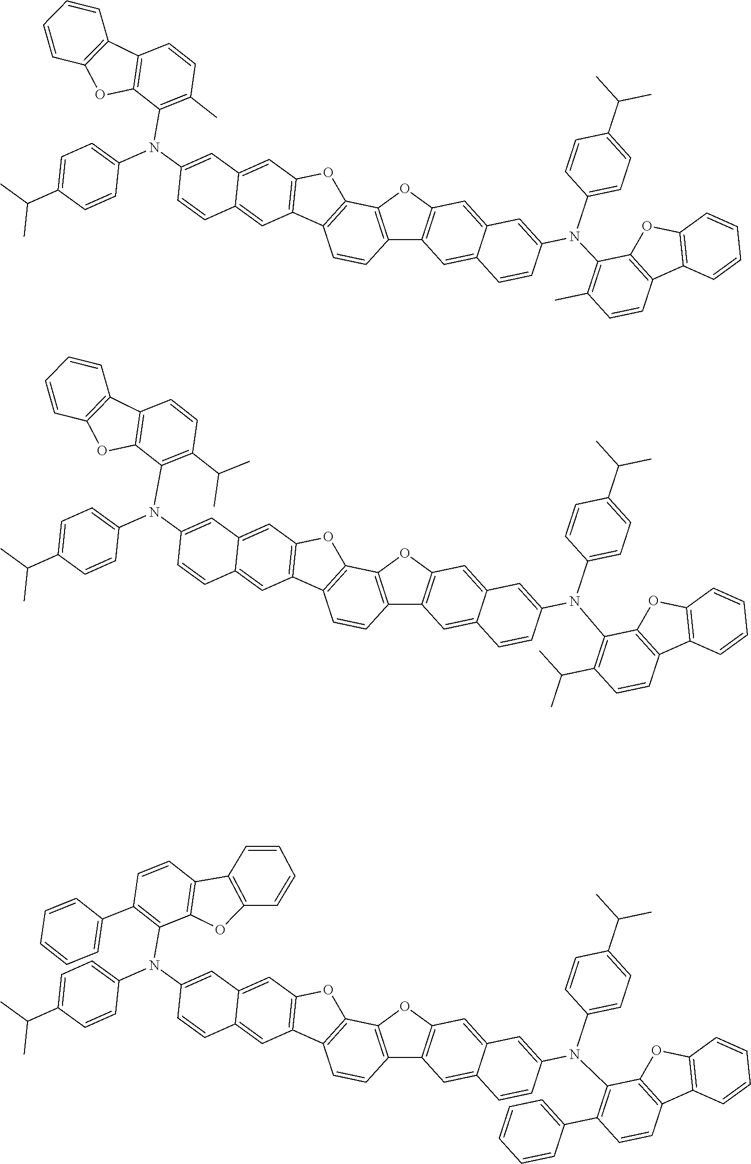 [SMILES: Cc1ccc2c(oc3ccccc32)c1N(c1ccc(C(C)C)cc1)c1ccc2cc3c(cc2c1)oc1c3ccc2c3cc4ccc(N(c5ccc(C(C)C)cc5)c5c(C)ccc6c5oc5ccccc56)cc4cc3oc21 ; CC(C)c1ccc(N(c2ccc3cc4c(cc3c2)oc2c4ccc3c4cc5ccc(N(c6ccc(C(C)C)cc6)c6c(C(C)C)ccc7c6oc6ccccc67)cc5cc4oc32)c2c(C(C)C)ccc3c2oc2ccccc23)cc1 ; CC(C)c1ccc(N(c2ccc3cc4c(cc3c2)oc2c4ccc3c4cc5ccc(N(c6ccc(C(C)C)cc6)c6c(-c7ccccc7)ccc7c6oc6ccccc67)cc5cc4oc32)c2c(-c3ccccc3)ccc3c2oc2ccccc23)cc1]

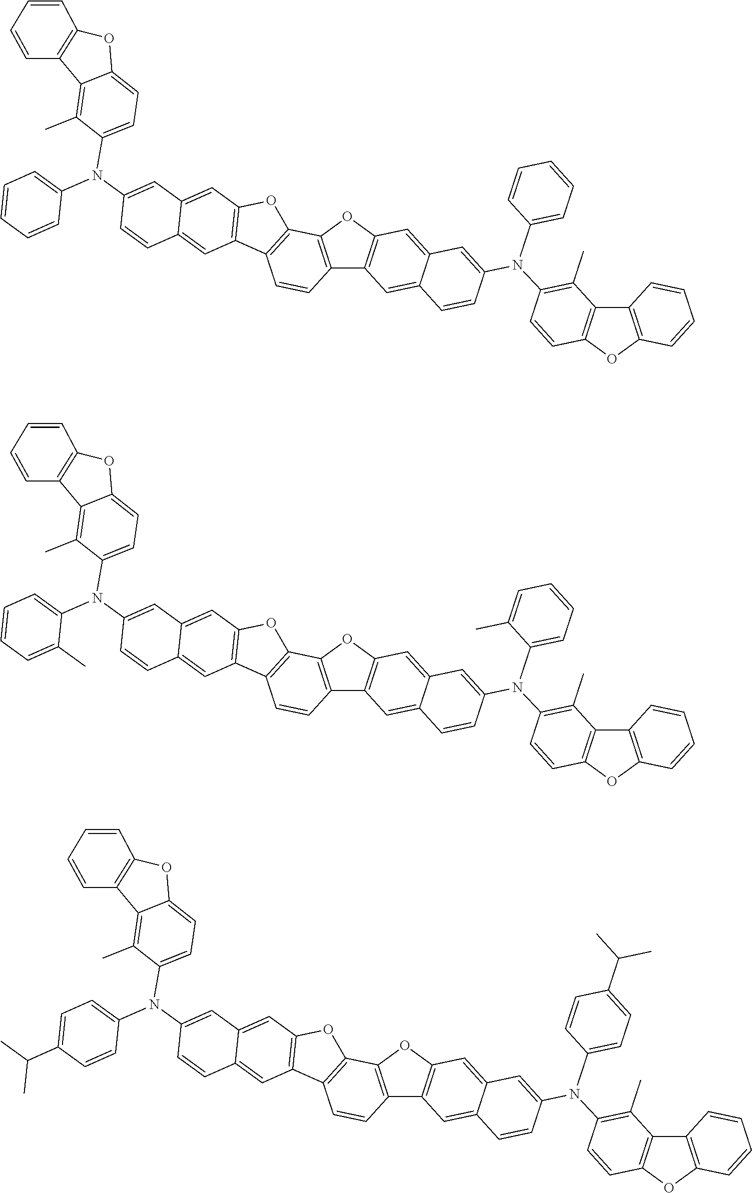 [SMILES: Cc1c(N(c2ccccc2)c2ccc3cc4c(cc3c2)oc2c4ccc3c4cc5ccc(N(c6ccccc6)c6ccc7oc8ccccc8c7c6C)cc5cc4oc32)ccc2oc3ccccc3c12 ; Cc1ccccc1N(c1ccc2cc3c(cc2c1)oc1c3ccc2c3cc4ccc(N(c5ccccc5C)c5ccc6oc7ccccc7c6c5C)cc4cc3oc21)c1ccc2oc3ccccc3c2c1C ; Cc1c(N(c2ccc(C(C)C)cc2)c2ccc3cc4c(cc3c2)oc2c4ccc3c4cc5ccc(N(c6ccc(C(C)C)cc6)c6ccc7oc8ccccc8c7c6C)cc5cc4oc32)ccc2oc3ccccc3c12]

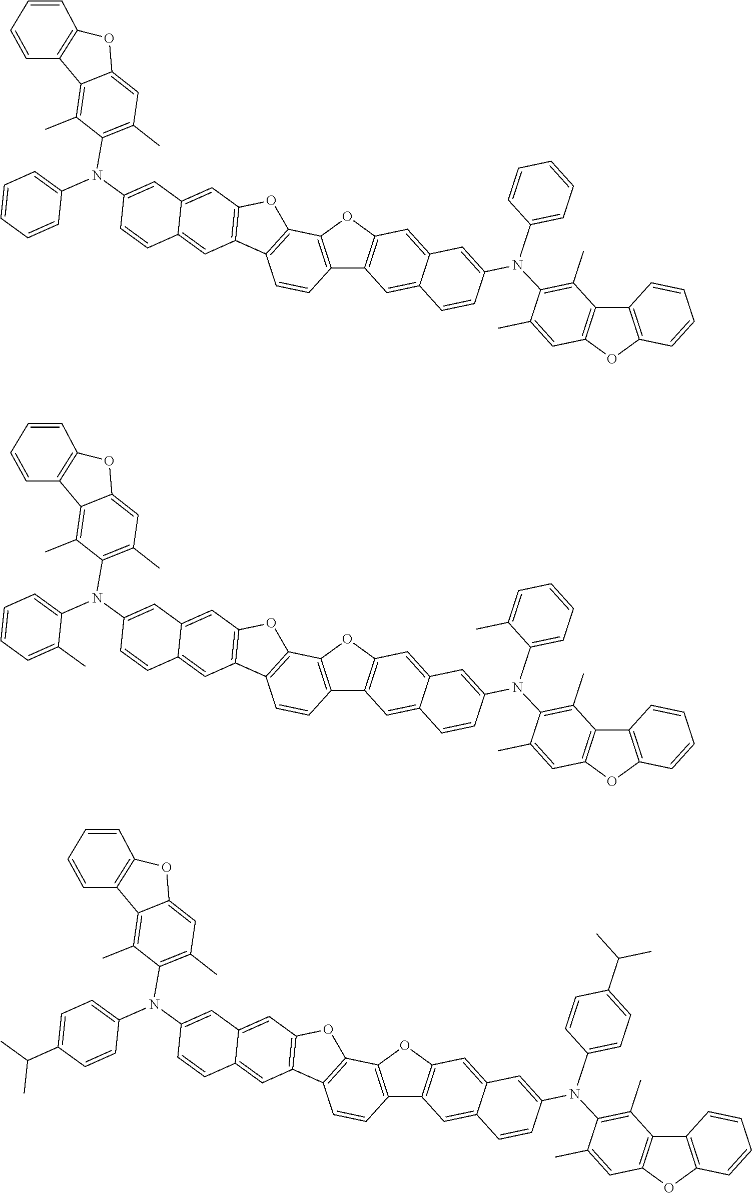 [SMILES: Cc1cc2oc3ccccc3c2c(C)c1N(c1ccccc1)c1ccc2cc3c(cc2c1)oc1c3ccc2c3cc4ccc(N(c5ccccc5)c5c(C)cc6oc7ccccc7c6c5C)cc4cc3oc21 ; Cc1ccccc1N(c1ccc2cc3c(cc2c1)oc1c3ccc2c3cc4ccc(N(c5ccccc5C)c5c(C)cc6oc7ccccc7c6c5C)cc4cc3oc21)c1c(C)cc2oc3ccccc3c2c1C ; Cc1cc2oc3ccccc3c2c(C)c1N(c1ccc(C(C)C)cc1)c1ccc2cc3c(cc2c1)oc1c3ccc2c3cc4ccc(N(c5ccc(C(C)C)cc5)c5c(C)cc6oc7ccccc7c6c5C)cc4cc3oc21]

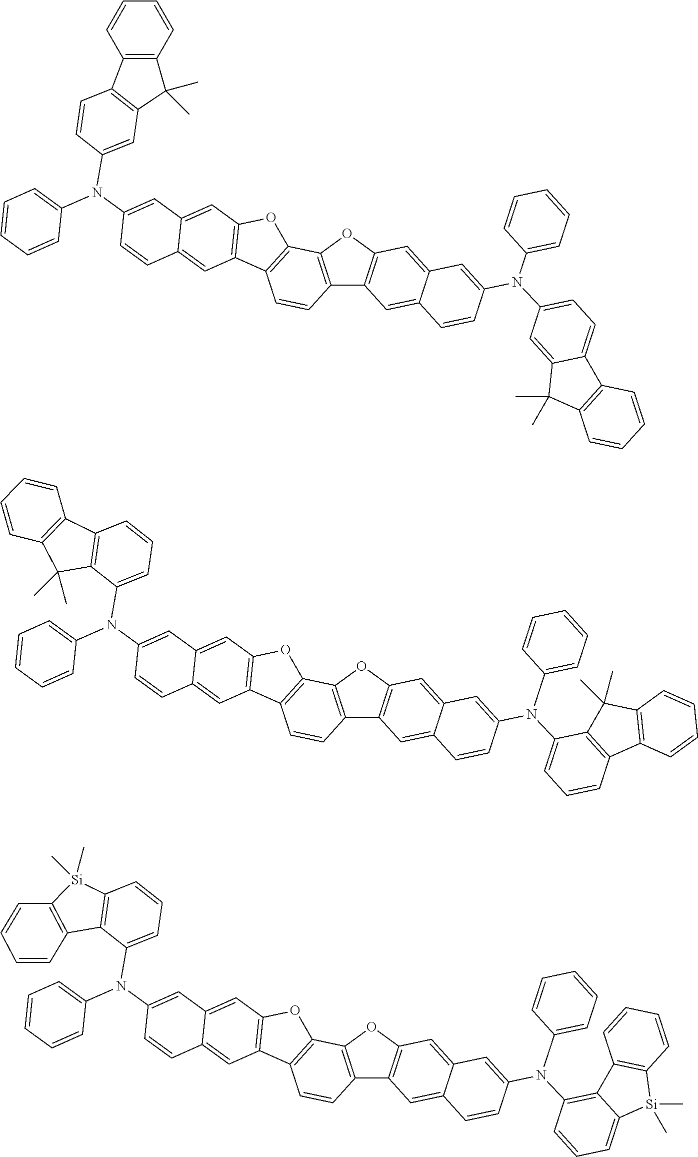 [SMILES: CC1(C)c2ccccc2-c2ccc(N(c3ccccc3)c3ccc4cc5c(cc4c3)oc3c5ccc4c5cc6ccc(N(c7ccccc7)c7ccc8c(c7)C(C)(C)c7ccccc7-8)cc6cc5oc43)cc21 ; CC1(C)c2ccccc2-c2cccc(N(c3ccccc3)c3ccc4cc5c(cc4c3)oc3c5ccc4c5cc6ccc(N(c7ccccc7)c7cccc8c7C(C)(C)c7ccccc7-8)cc6cc5oc43)c21 ; C[Si]1(C)c2ccccc2-c2c(N(c3ccccc3)c3ccc4cc5c(cc4c3)oc3c5ccc4c5cc6ccc(N(c7ccccc7)c7cccc8c7-c7ccccc7[Si]8(C)C)cc6cc5oc43)cccc21]

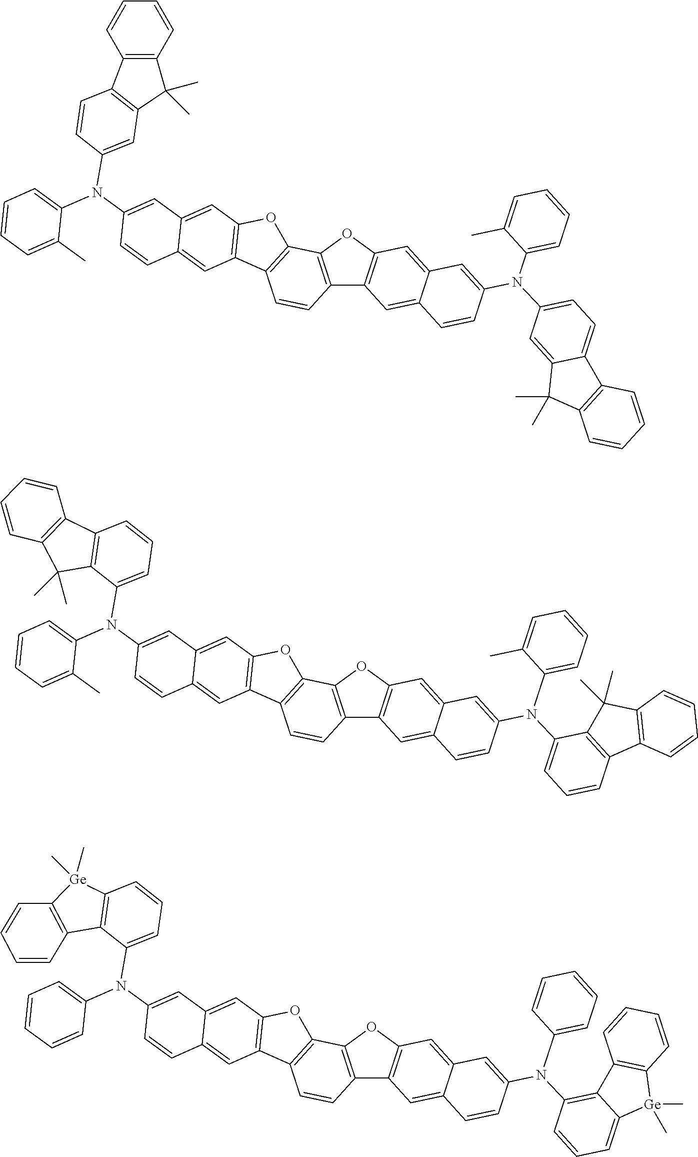

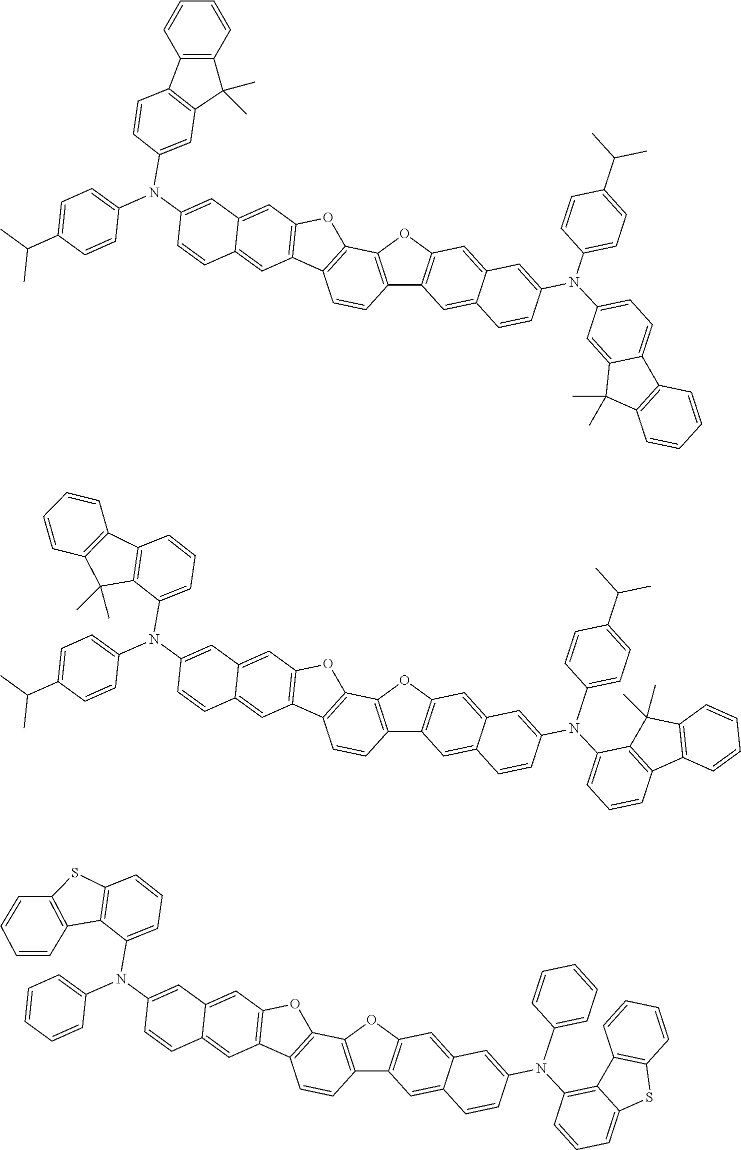 [SMILES: CC(C)c1ccc(N(c2ccc3c(c2)C(C)(C)c2ccccc2-3)c2ccc3cc4c(cc3c2)oc2c4ccc3c4cc5ccc(N(c6ccc(C(C)C)cc6)c6ccc7c(c6)C(C)(C)c6ccccc6-7)cc5cc4oc32)cc1 ; CC(C)c1ccc(N(c2ccc3cc4c(cc3c2)oc2c4ccc3c4cc5ccc(N(c6ccc(C(C)C)cc6)c6cccc7c6C(C)(C)c6ccccc6-7)cc5cc4oc32)c2cccc3c2C(C)(C)c2ccccc2-3)cc1 ; c1ccc(N(c2ccc3cc4c(cc3c2)oc2c4ccc3c4cc5ccc(N(c6ccccc6)c6cccc7sc8ccccc8c67)cc5cc4oc32)c2cccc3sc4ccccc4c23)cc1]

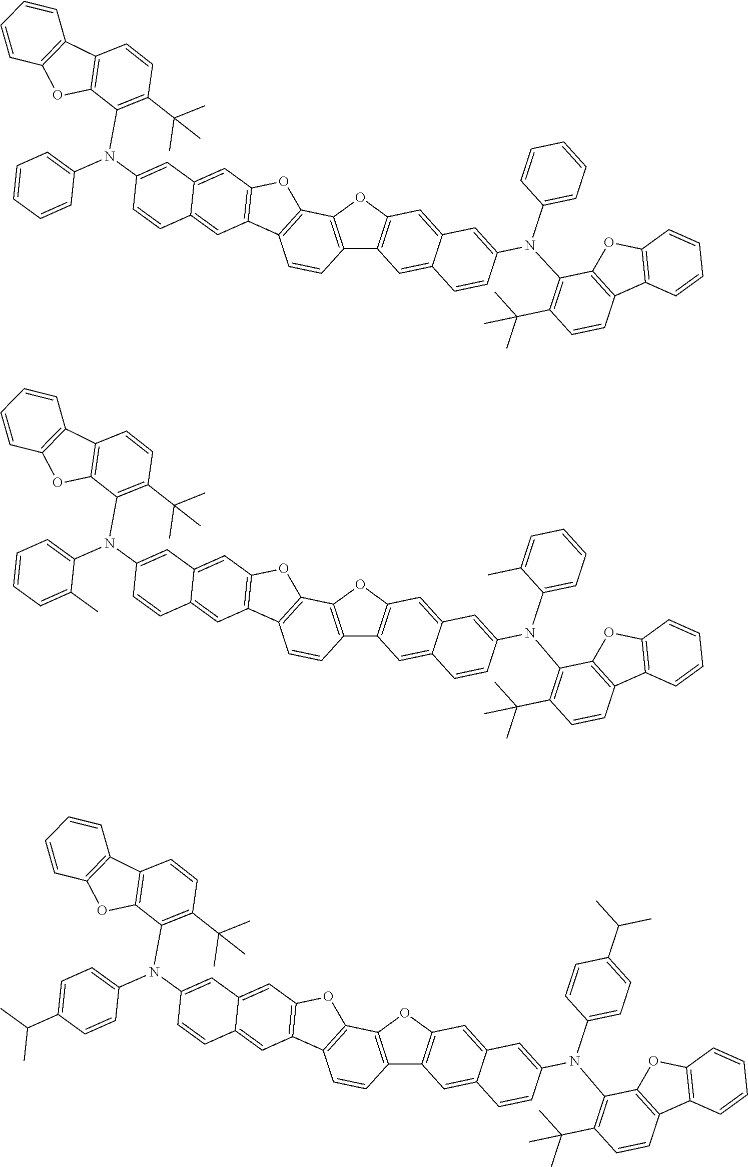 [SMILES: CC(C)(C)c1ccc2c(oc3ccccc32)c1N(c1ccccc1)c1ccc2cc3c(cc2c1)oc1c3ccc2c3cc4ccc(N(c5ccccc5)c5c(C(C)(C)C)ccc6c5oc5ccccc56)cc4cc3oc21 ; Cc1ccccc1N(c1ccc2cc3c(cc2c1)oc1c3ccc2c3cc4ccc(N(c5ccccc5C)c5c(C(C)(C)C)ccc6c5oc5ccccc56)cc4cc3oc21)c1c(C(C)(C)C)ccc2c1oc1ccccc12 ; CC(C)c1ccc(N(c2ccc3cc4c(cc3c2)oc2c4ccc3c4cc5ccc(N(c6ccc(C(C)C)cc6)c6c(C(C)(C)C)ccc7c6oc6ccccc67)cc5cc4oc32)c2c(C(C)(C)C)ccc3c2oc2ccccc23)cc1]

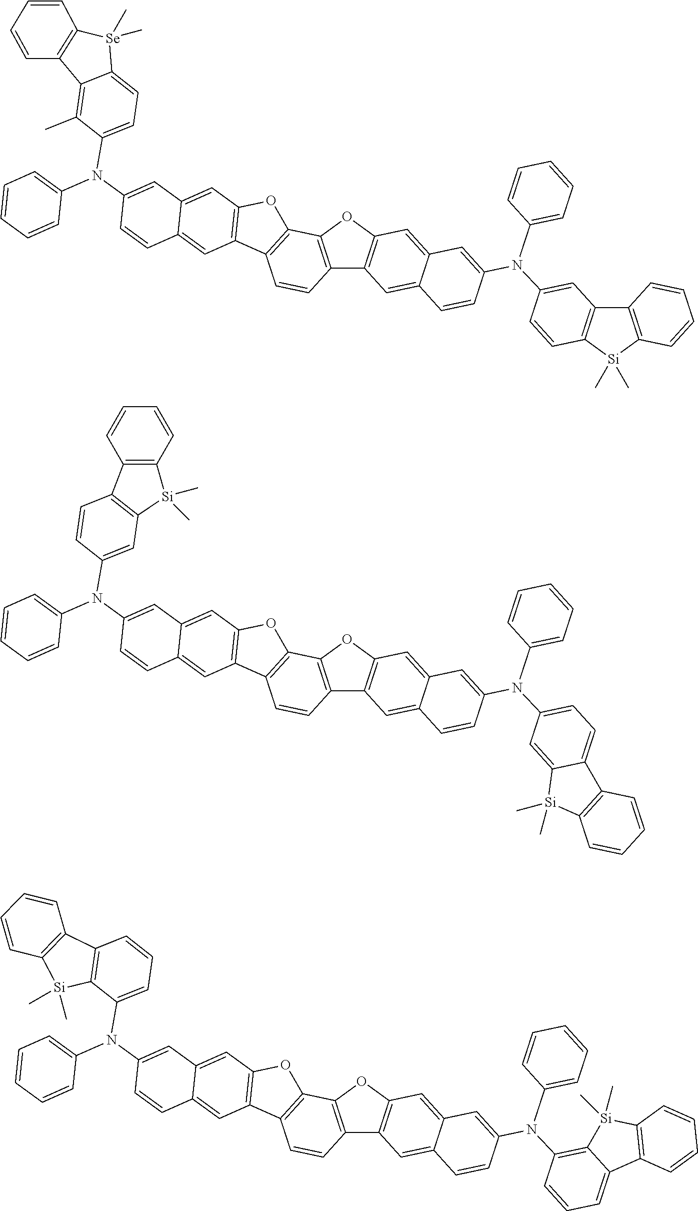 [SMILES: Cc1c(N(c2ccccc2)c2ccc3cc4c(cc3c2)oc2c4ccc3c4cc5ccc(N(c6ccccc6)c6ccc7c(c6)-c6ccccc6[Si]7(C)C)cc5cc4oc32)ccc2c1-c1ccccc1[Se]2(C)C ; C[Si]1(C)c2ccccc2-c2ccc(N(c3ccccc3)c3ccc4cc5c(cc4c3)oc3c5ccc4c5cc6ccc(N(c7ccccc7)c7ccc8c(c7)[Si](C)(C)c7ccccc7-8)cc6cc5oc43)cc21 ; C[Si]1(C)c2ccccc2-c2cccc(N(c3ccccc3)c3ccc4cc5c(cc4c3)oc3c5ccc4c5cc6ccc(N(c7ccccc7)c7cccc8c7[Si](C)(C)c7ccccc7-8)cc6cc5oc43)c21]

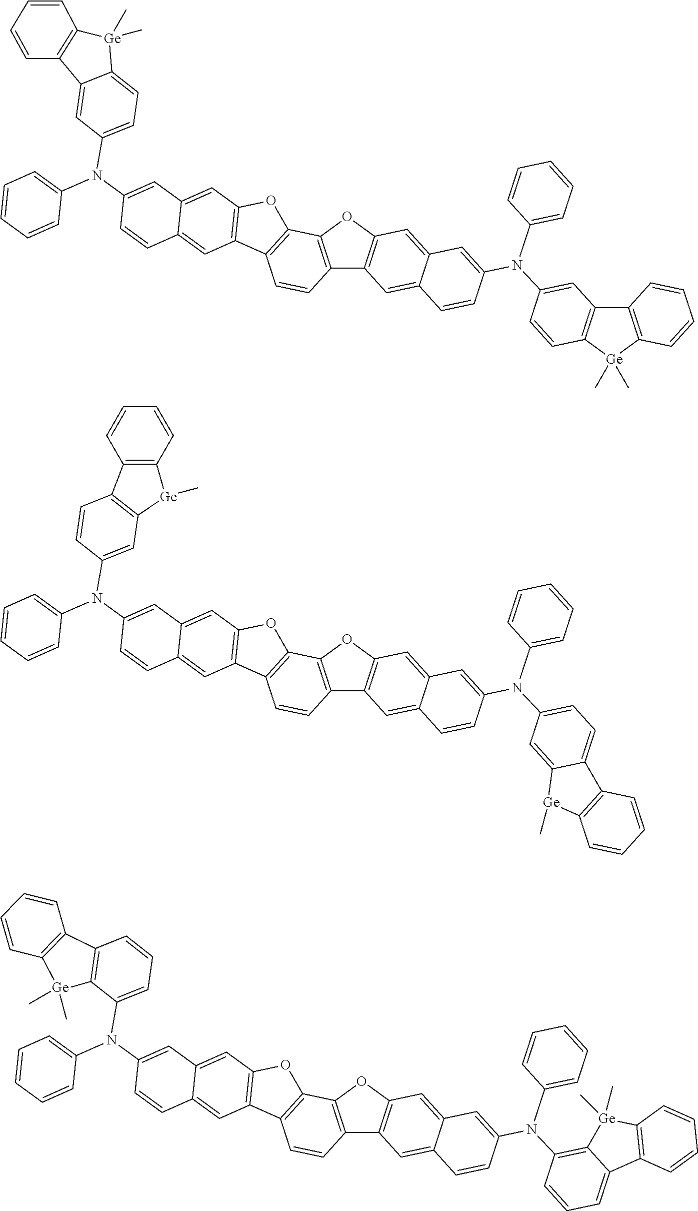

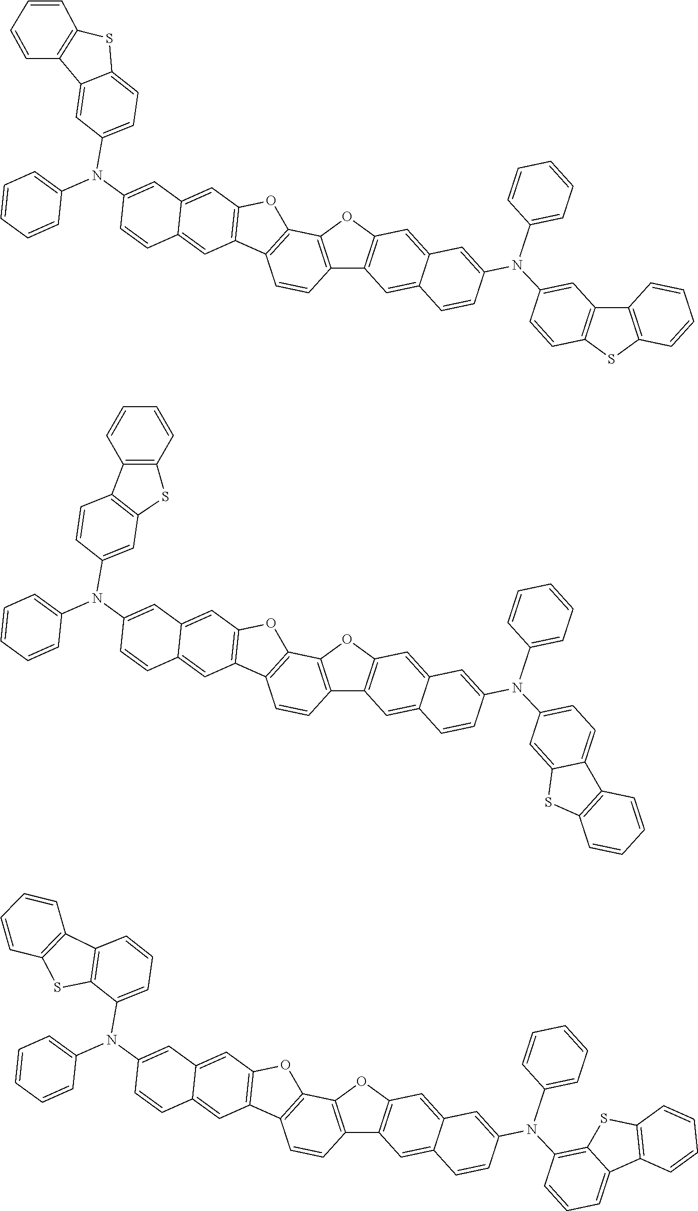 [SMILES: c1ccc(N(c2ccc3cc4c(cc3c2)oc2c4ccc3c4cc5ccc(N(c6ccccc6)c6ccc7sc8ccccc8c7c6)cc5cc4oc32)c2ccc3sc4ccccc4c3c2)cc1 ; c1ccc(N(c2ccc3cc4c(cc3c2)oc2c4ccc3c4cc5ccc(N(c6ccccc6)c6ccc7c(c6)sc6ccccc67)cc5cc4oc32)c2ccc3c(c2)sc2ccccc23)cc1 ; c1ccc(N(c2ccc3cc4c(cc3c2)oc2c4ccc3c4cc5ccc(N(c6ccccc6)c6cccc7c6sc6ccccc67)cc5cc4oc32)c2cccc3c2sc2ccccc23)cc1]

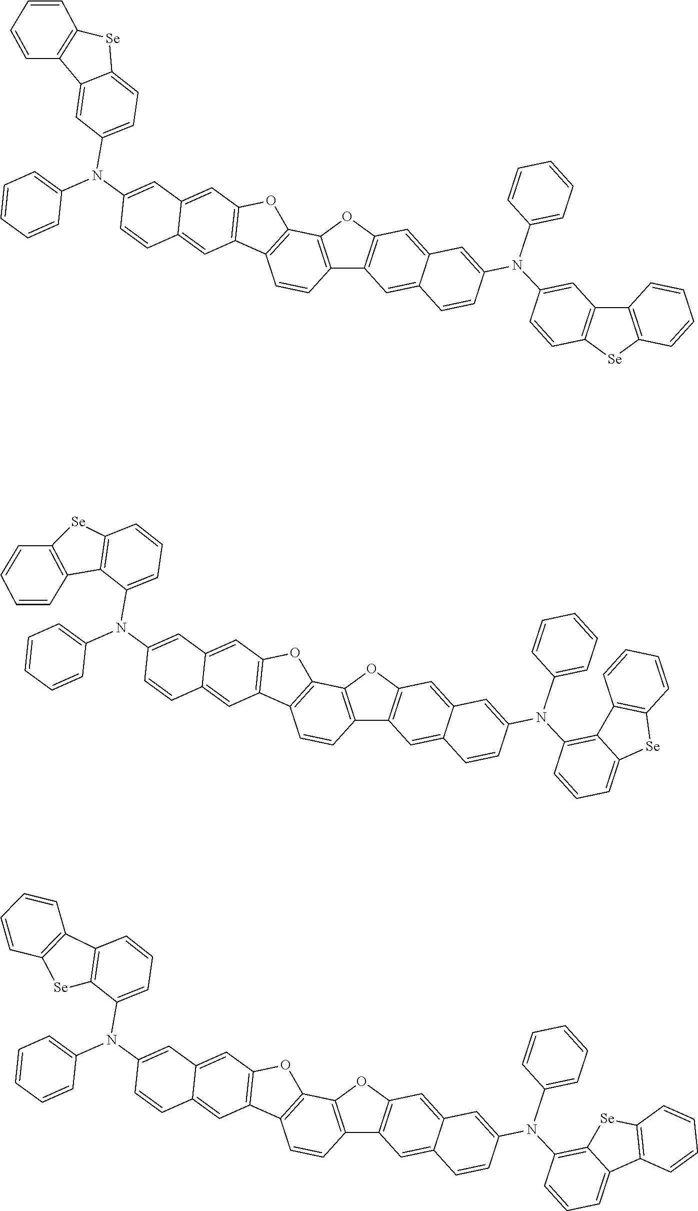 [SMILES: c1ccc(N(c2ccc3cc4c(cc3c2)oc2c4ccc3c4cc5ccc(N(c6ccccc6)c6ccc7[se]c8ccccc8c7c6)cc5cc4oc32)c2ccc3[se]c4ccccc4c3c2)cc1 ; c1ccc(N(c2ccc3cc4c(cc3c2)oc2c4ccc3c4cc5ccc(N(c6ccccc6)c6cccc7[se]c8ccccc8c67)cc5cc4oc32)c2cccc3[se]c4ccccc4c23)cc1 ; c1ccc(N(c2ccc3cc4c(cc3c2)oc2c4ccc3c4cc5ccc(N(c6ccccc6)c6cccc7c6[se]c6ccccc67)cc5cc4oc32)c2cccc3c2[se]c2ccccc23)cc1]

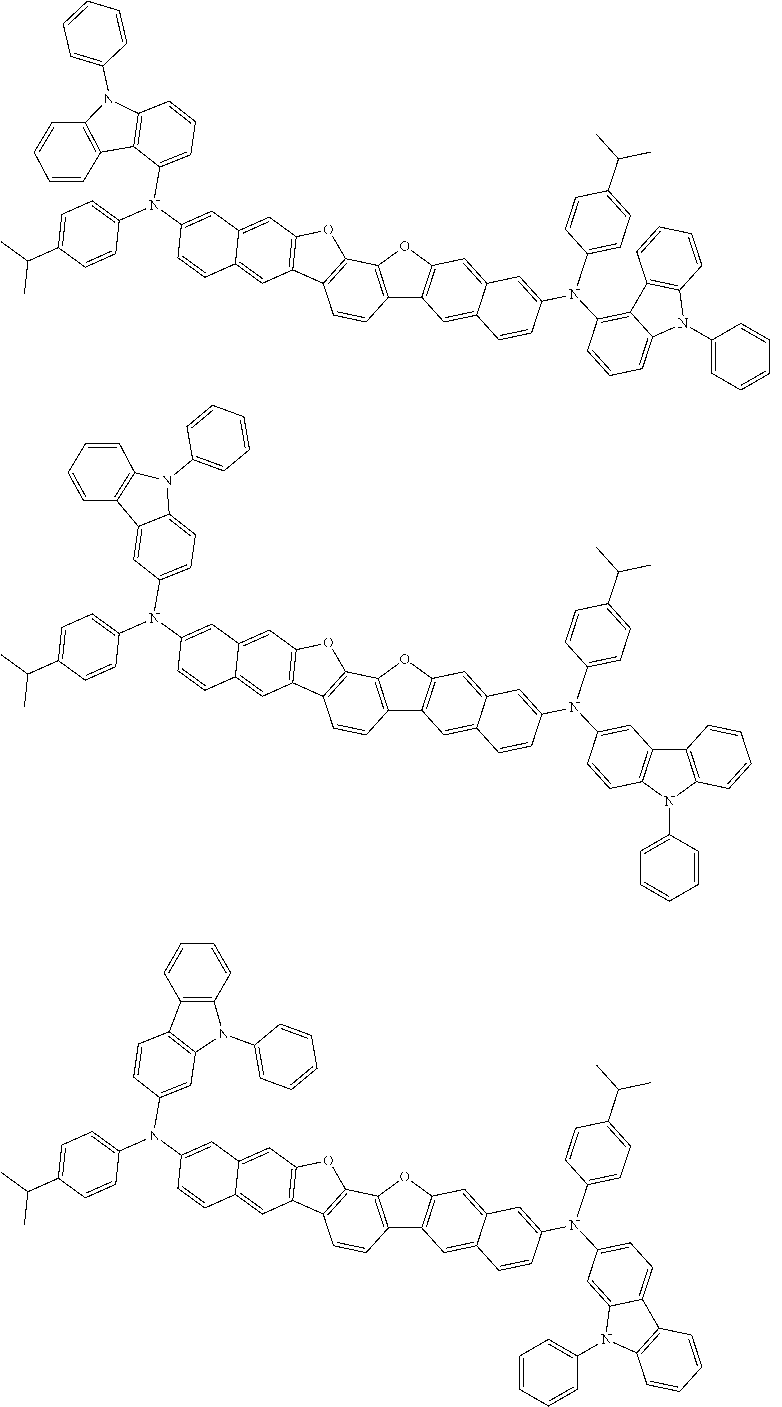

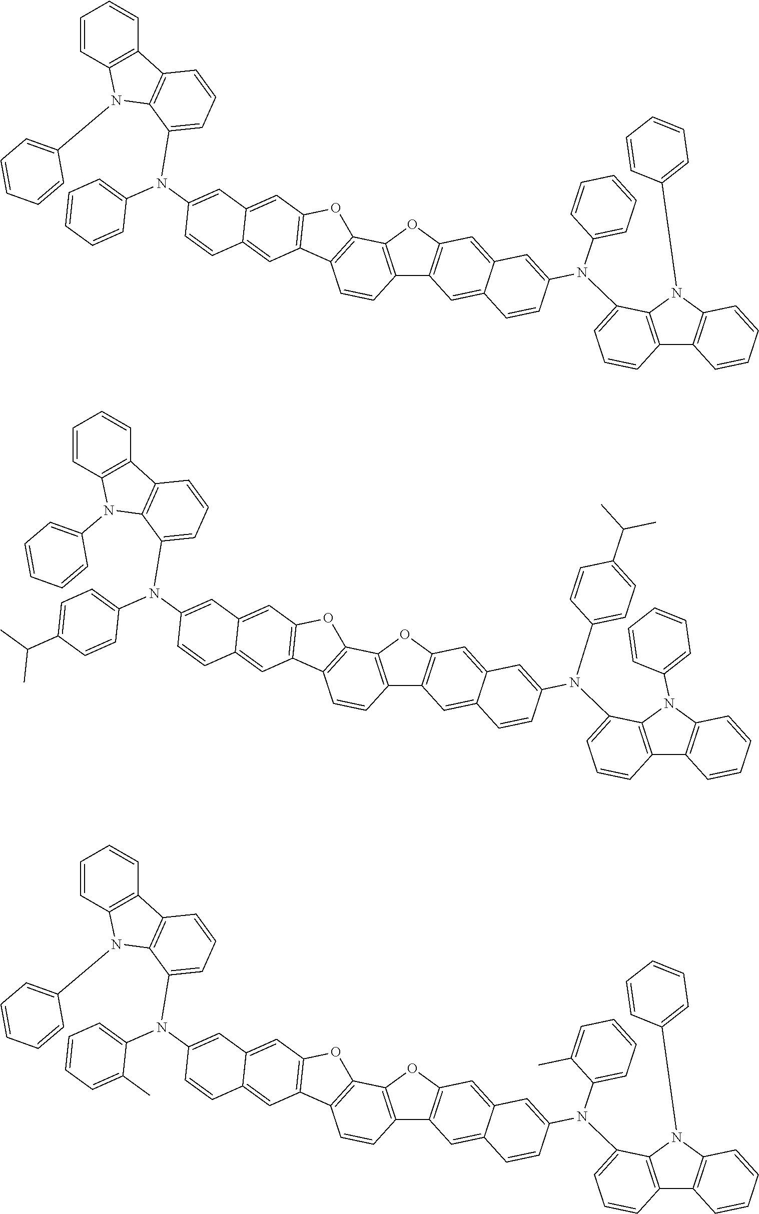

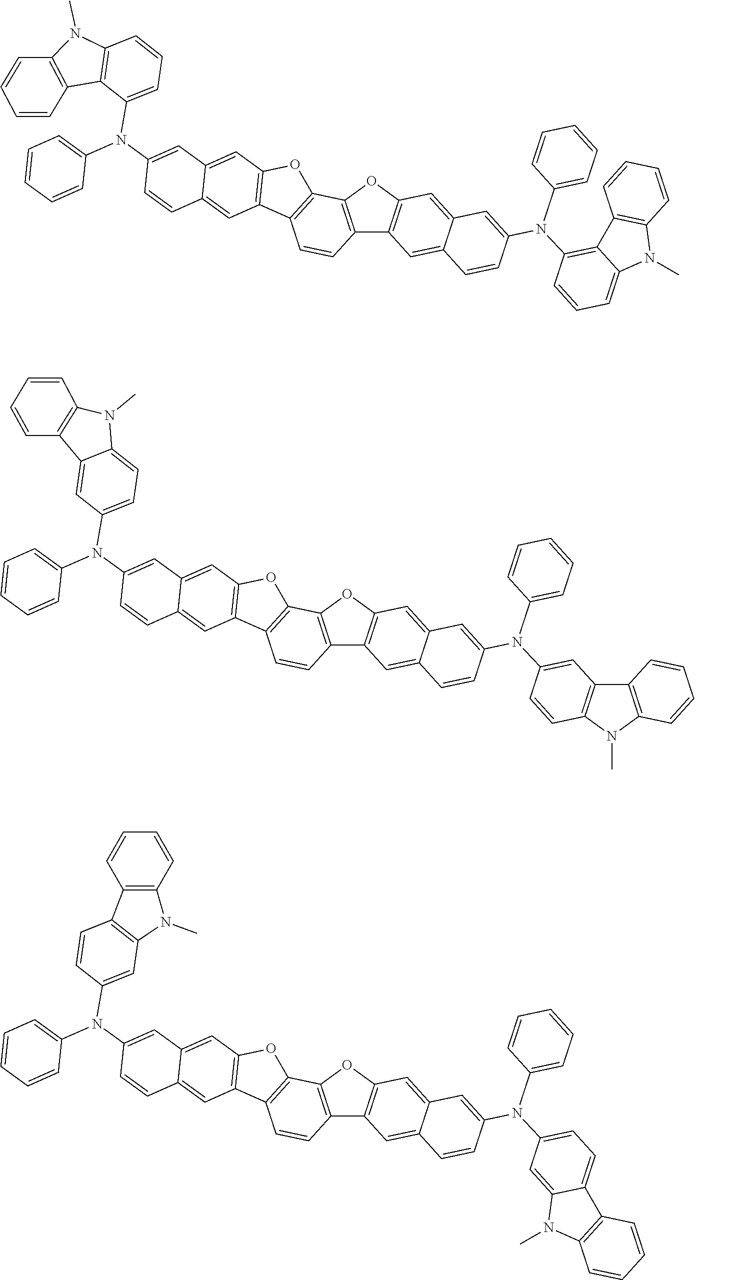

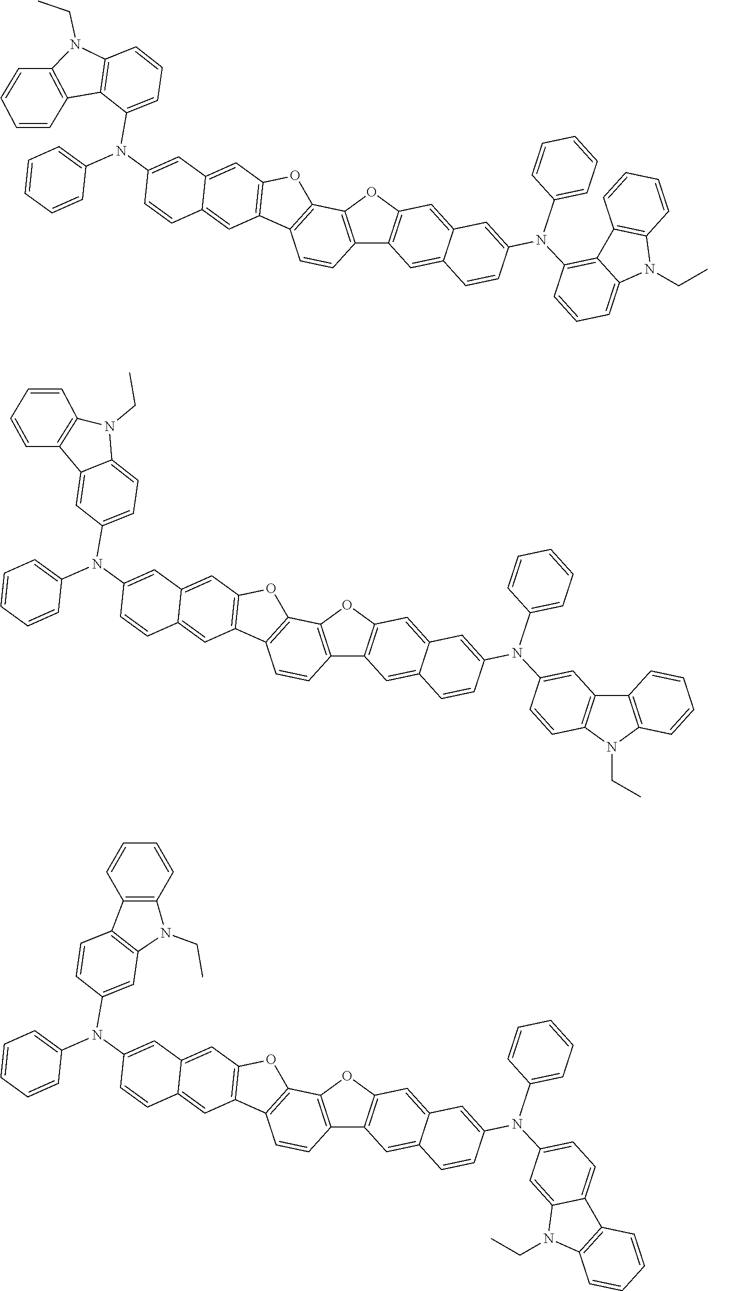

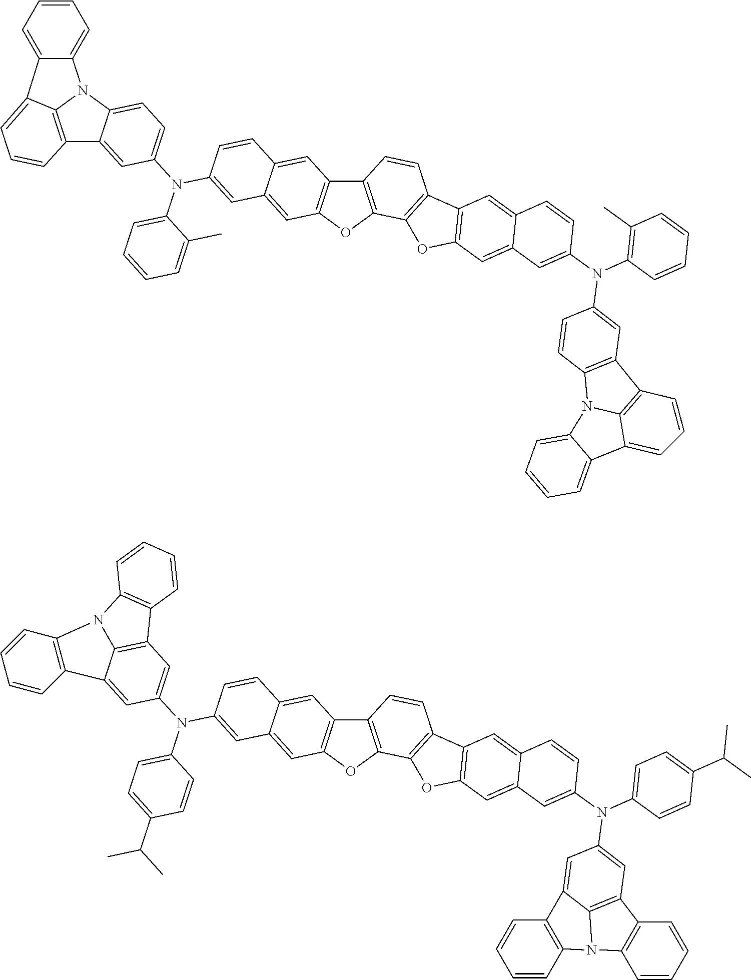 [SMILES: Cc1ccccc1N(c1ccc2cc3c(cc2c1)oc1c3ccc2c3cc4ccc(N(c5ccc6c(c5)c5cccc7c8ccccc8n6c75)c5ccccc5C)cc4cc3oc21)c1ccc2c(c1)c1cccc3c4ccccc4n2c31 ; CC(C)c1ccc(N(c2ccc3cc4c(cc3c2)oc2c4ccc3c4cc5ccc(N(c6ccc(C(C)C)cc6)c6cc7c8ccccc8n8c9ccccc9c(c6)c78)cc5cc4oc32)c2cc3c4ccccc4n4c5ccccc5c(c2)c34)cc1]

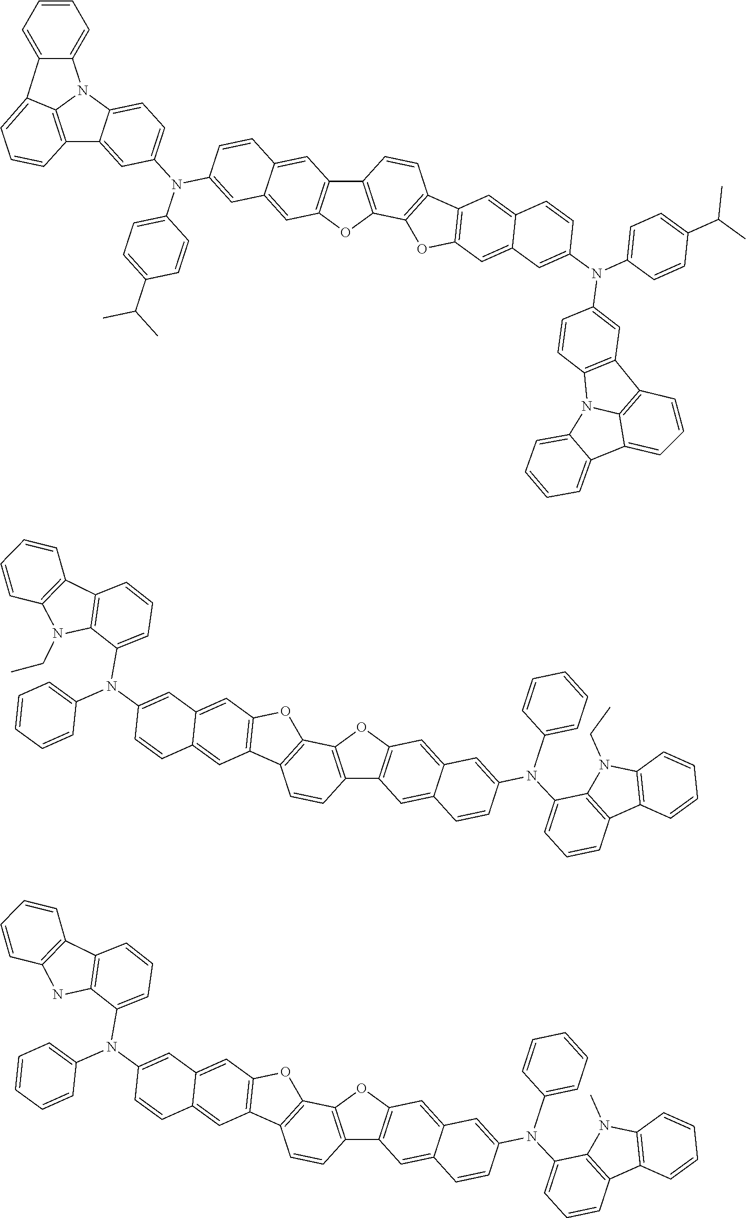 [SMILES: CC(C)c1ccc(N(c2ccc3cc4c(cc3c2)oc2c4ccc3c4cc5ccc(N(c6ccc(C(C)C)cc6)c6ccc7c(c6)c6cccc8c9ccccc9n7c86)cc5cc4oc32)c2ccc3c(c2)c2cccc4c5ccccc5n3c42)cc1 ; CCn1c2ccccc2c2cccc(N(c3ccccc3)c3ccc4cc5c(cc4c3)oc3c5ccc4c5cc6ccc(N(c7ccccc7)c7cccc8c9ccccc9n(CC)c78)cc6cc5oc43)c21 ; Cn1c2ccccc2c2cccc(N(c3ccccc3)c3ccc4cc5c(cc4c3)oc3c5ccc4c5cc6ccc(N(c7ccccc7)c7cccc8c7[nH]c7ccccc78)cc6cc5oc43)c21]

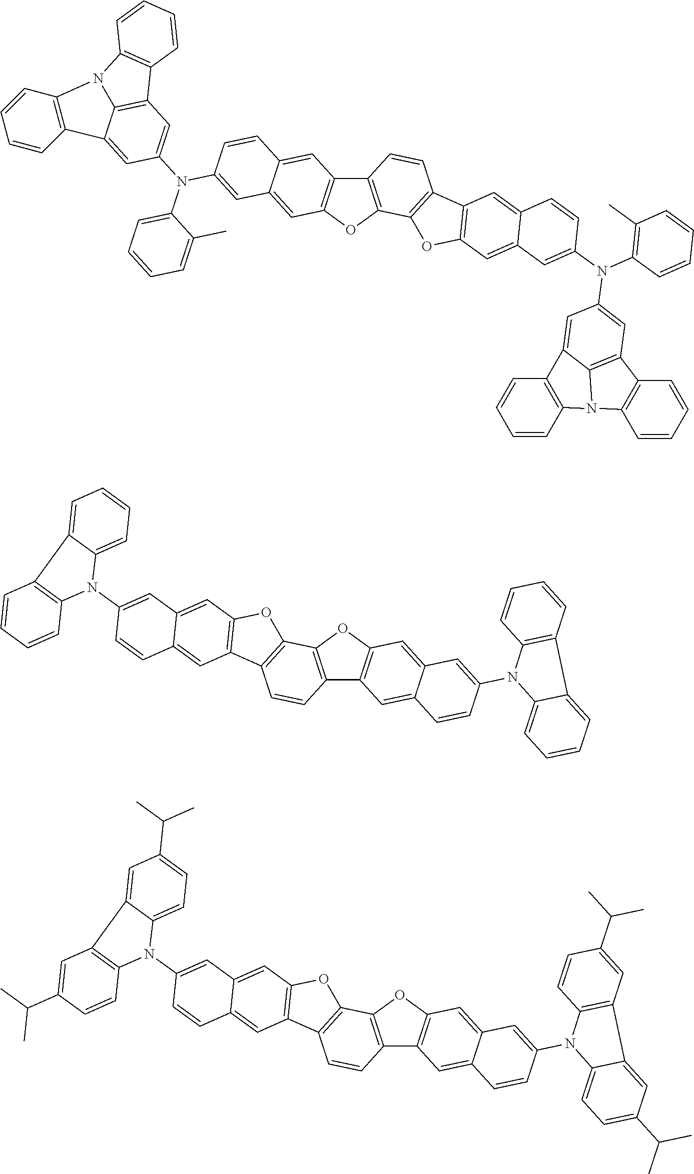 [SMILES: Cc1ccccc1N(c1ccc2cc3c(cc2c1)oc1c3ccc2c3cc4ccc(N(c5cc6c7ccccc7n7c8ccccc8c(c5)c67)c5ccccc5C)cc4cc3oc21)c1cc2c3ccccc3n3c4ccccc4c(c1)c23 ; c1ccc2c(c1)c1ccccc1n2-c1ccc2cc3c(cc2c1)oc1c3ccc2c3cc4ccc(-n5c6ccccc6c6ccccc65)cc4cc3oc21 ; CC(C)c1ccc2c(c1)c1cc(C(C)C)ccc1n2-c1ccc2cc3c(cc2c1)oc1c3ccc2c3cc4ccc(-n5c6ccc(C(C)C)cc6c6cc(C(C)C)ccc65)cc4cc3oc21]

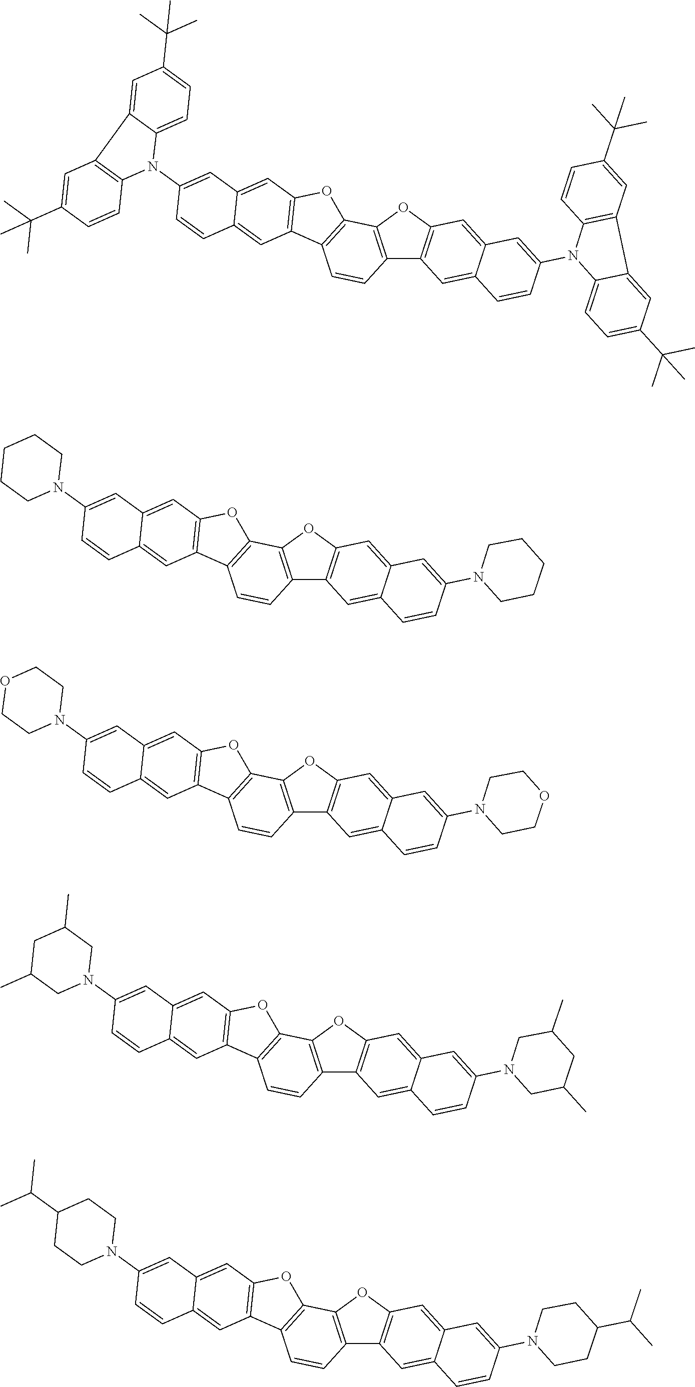

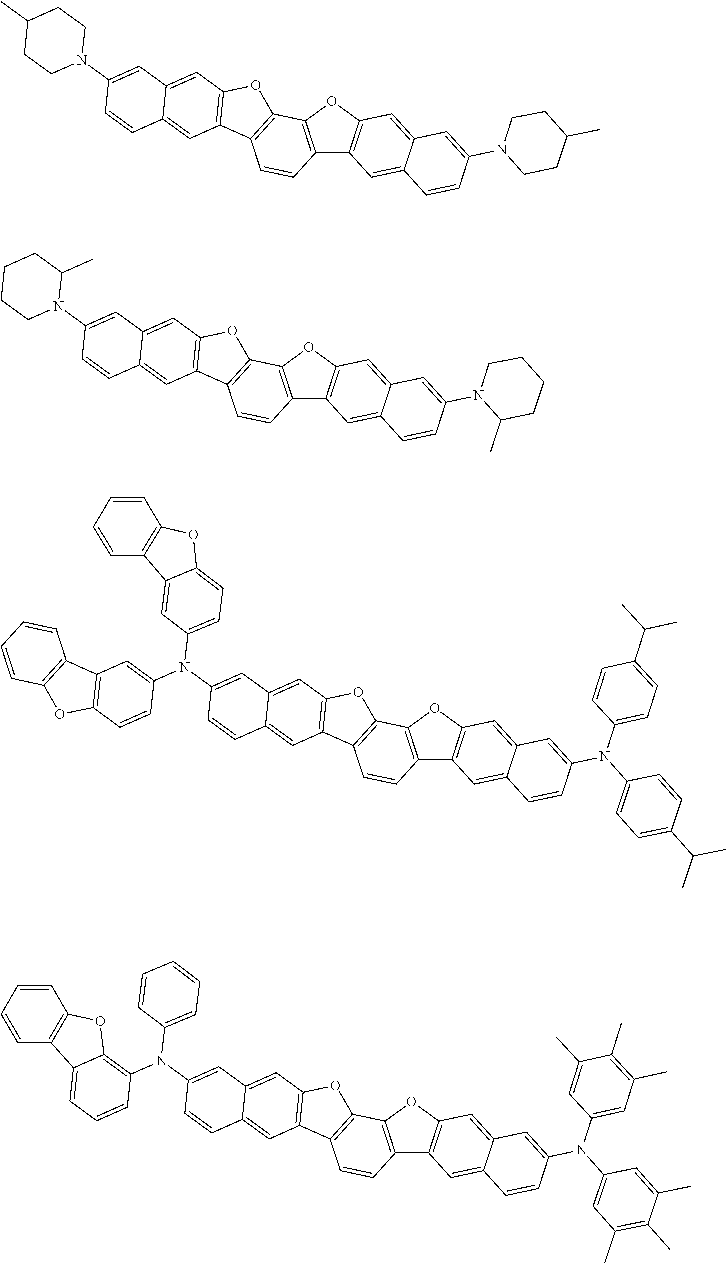

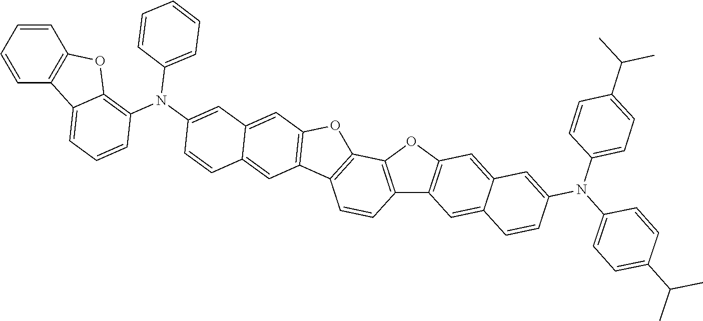 [SMILES: CC(C)c1ccc(N(c2ccc(C(C)C)cc2)c2ccc3cc4c(cc3c2)oc2c4ccc3c4cc5ccc(N(c6ccccc6)c6cccc7c6oc6ccccc67)cc5cc4oc32)cc1]

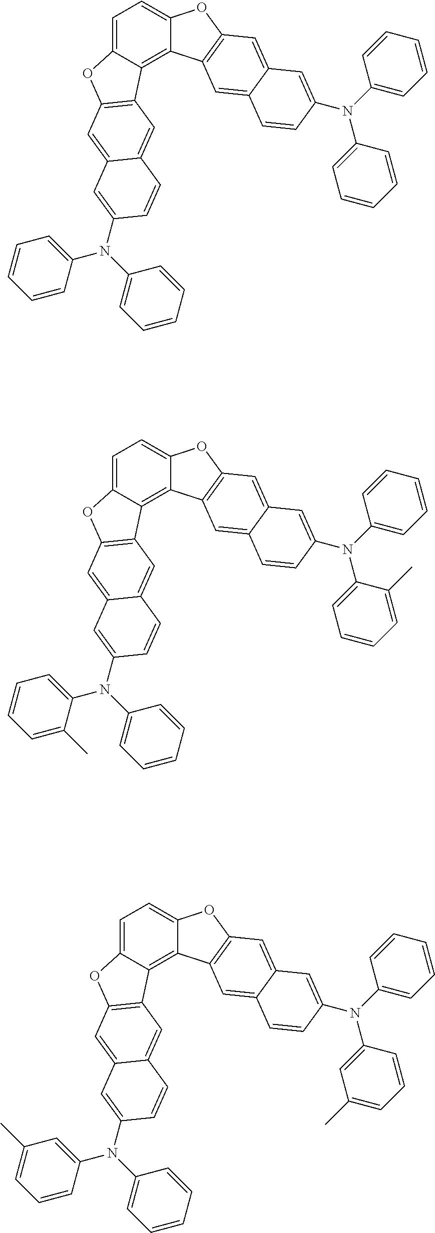 [SMILES: c1ccc(N(c2ccccc2)c2ccc3cc4c(cc3c2)oc2ccc3oc5cc6cc(N(c7ccccc7)c7ccccc7)ccc6cc5c3c24)cc1 ; Cc1ccccc1N(c1ccccc1)c1ccc2cc3c(cc2c1)oc1ccc2oc4cc5cc(N(c6ccccc6)c6ccccc6C)ccc5cc4c2c13 ; Cc1cccc(N(c2ccccc2)c2ccc3cc4c(cc3c2)oc2ccc3oc5cc6cc(N(c7ccccc7)c7cccc(C)c7)ccc6cc5c3c24)c1]

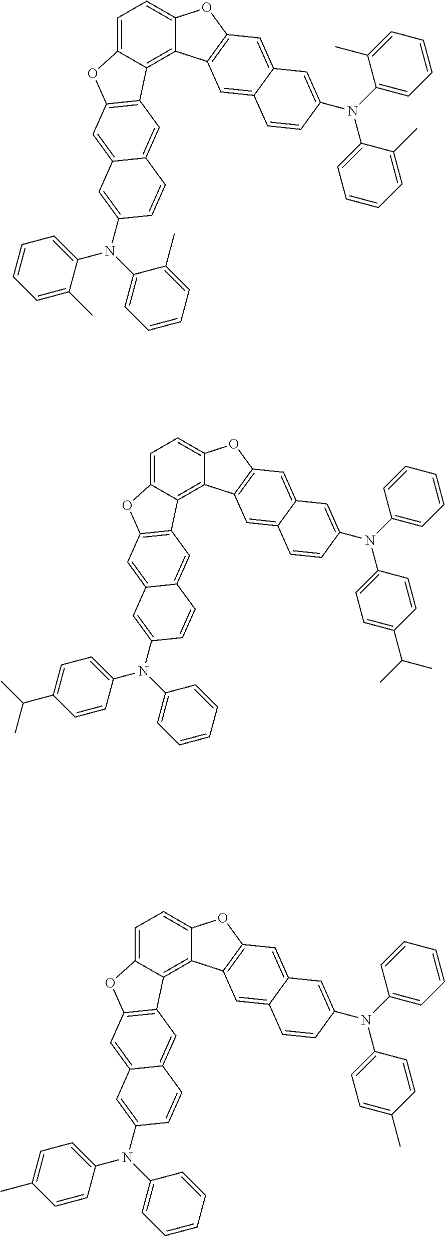 [SMILES: Cc1ccccc1N(c1ccc2cc3c(cc2c1)oc1ccc2oc4cc5cc(N(c6ccccc6C)c6ccccc6C)ccc5cc4c2c13)c1ccccc1C ; CC(C)c1ccc(N(c2ccccc2)c2ccc3cc4c(cc3c2)oc2ccc3oc5cc6cc(N(c7ccccc7)c7ccc(C(C)C)cc7)ccc6cc5c3c24)cc1 ; Cc1ccc(N(c2ccccc2)c2ccc3cc4c(cc3c2)oc2ccc3oc5cc6cc(N(c7ccccc7)c7ccc(C)cc7)ccc6cc5c3c24)cc1]

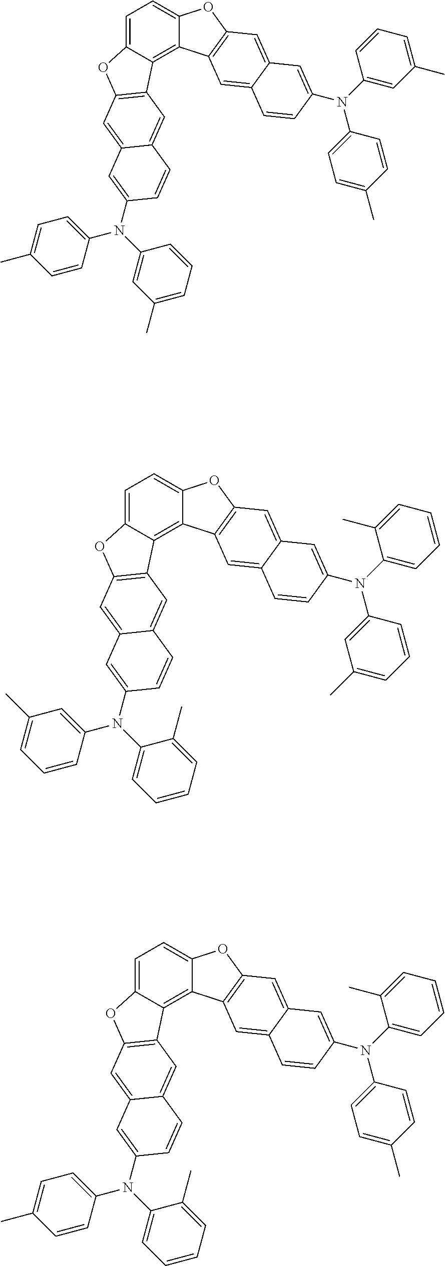

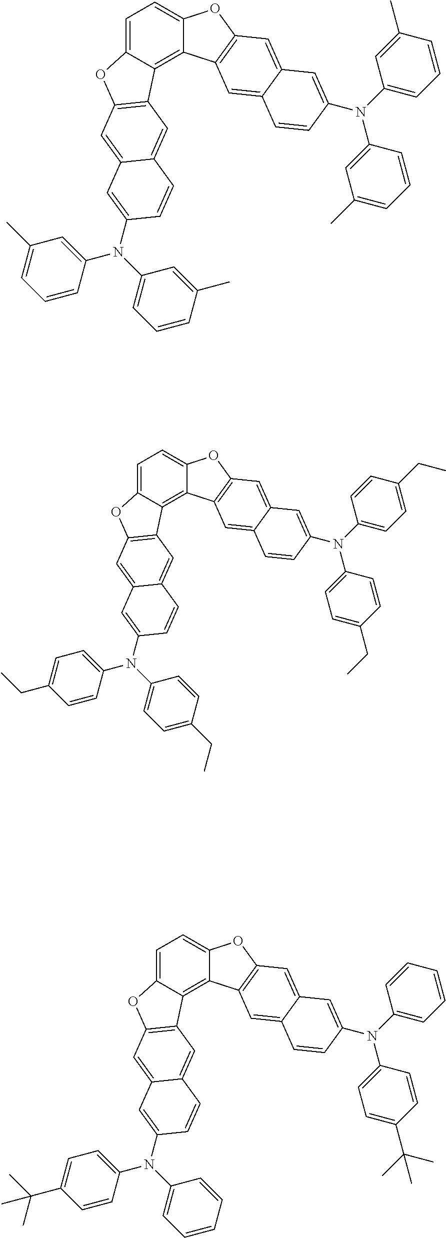

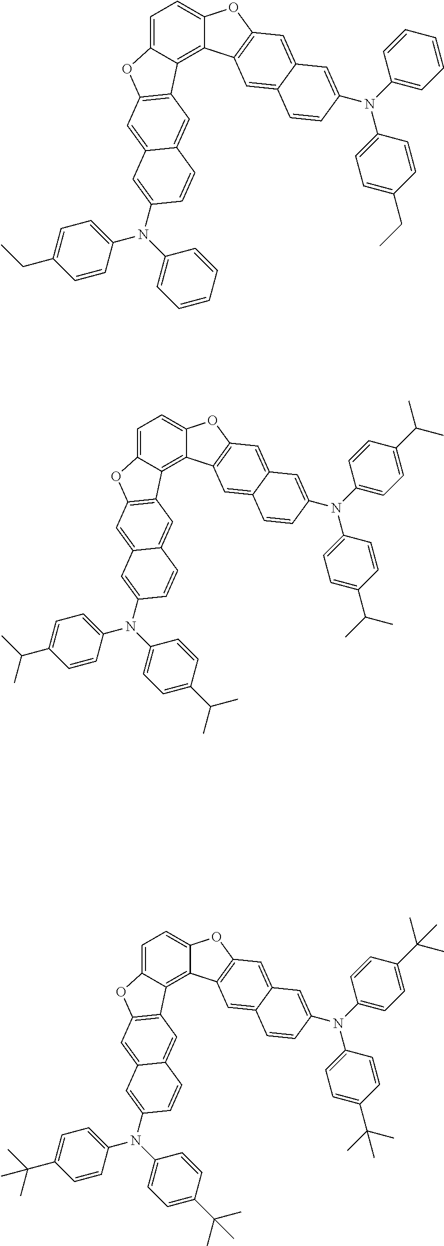

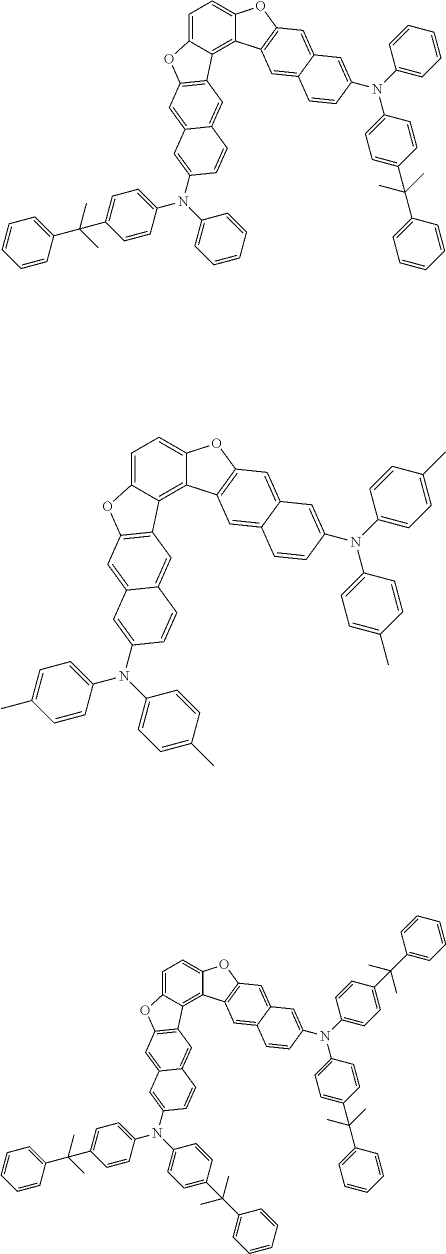

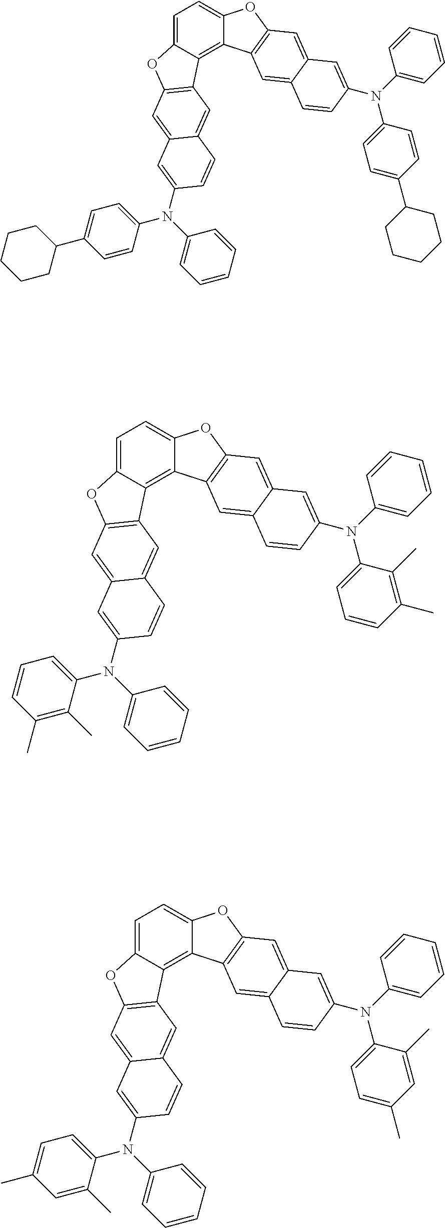

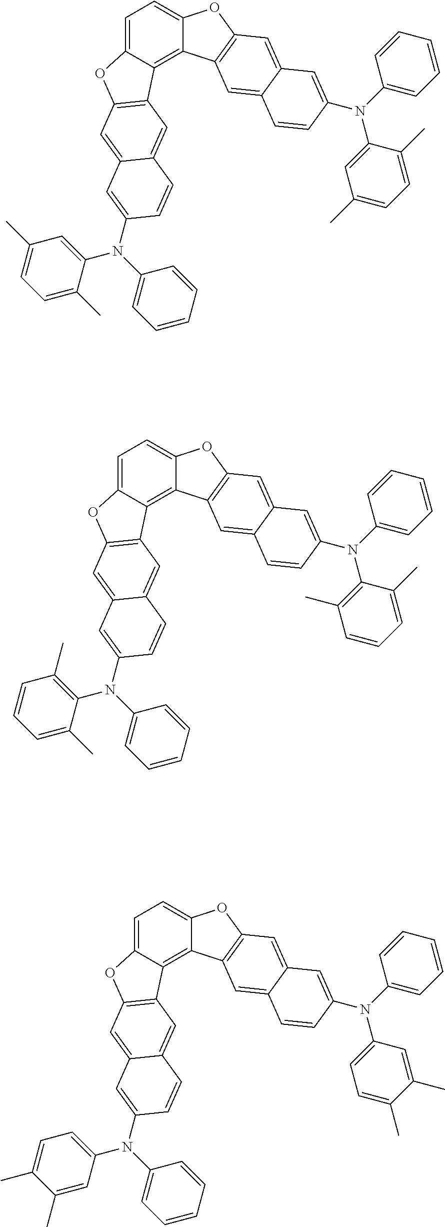

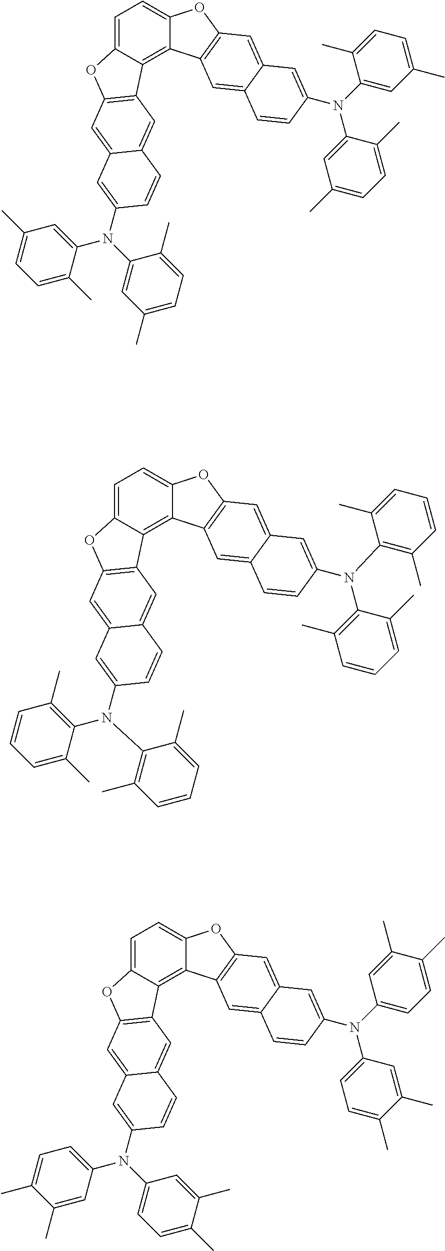

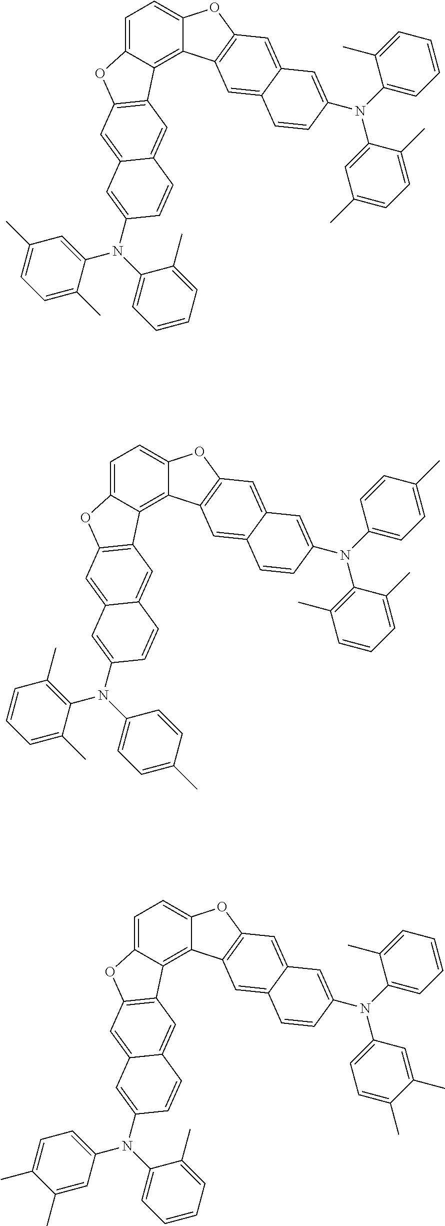

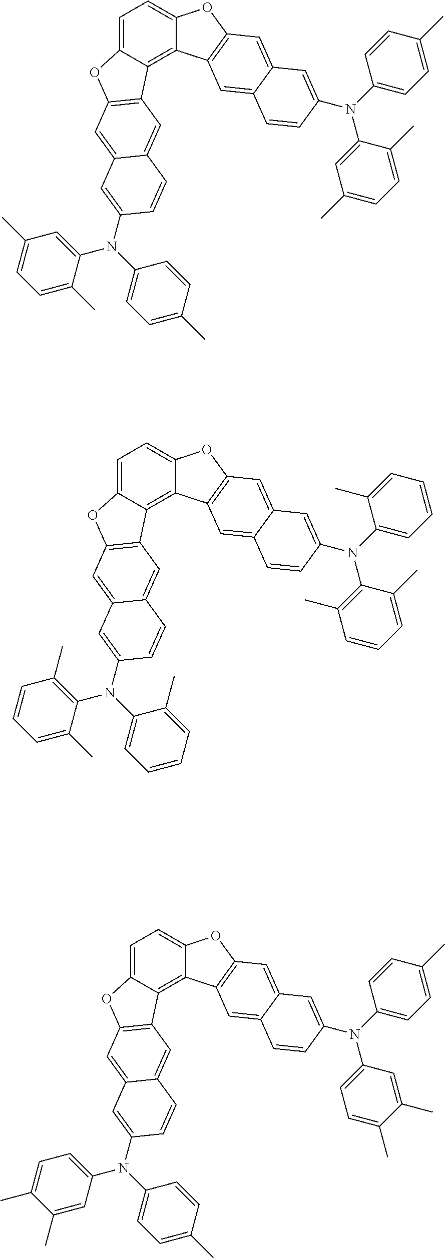

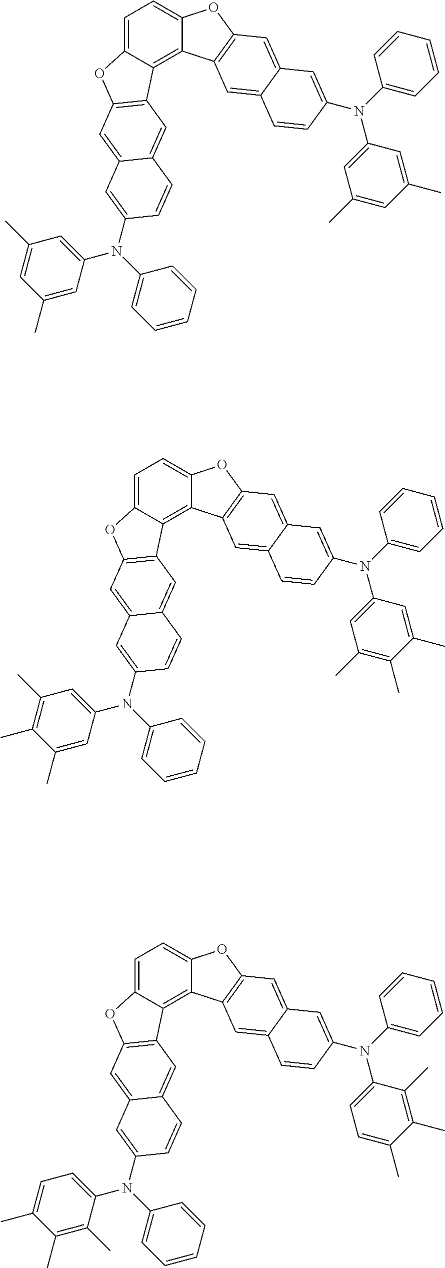

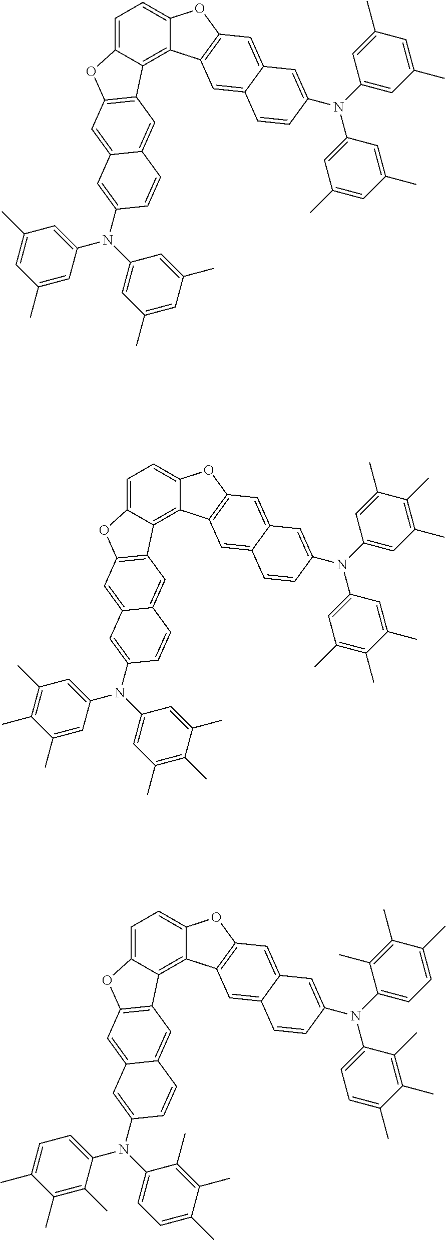

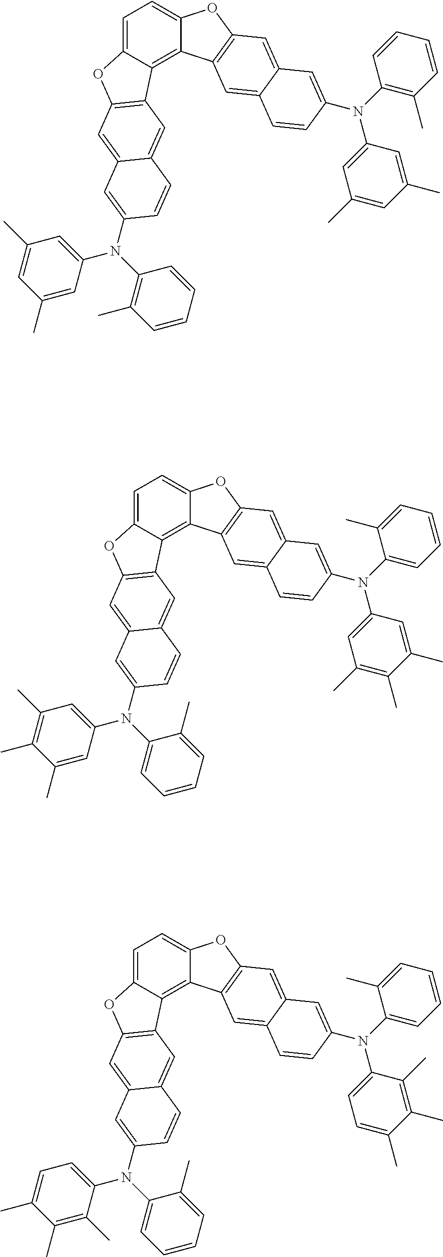

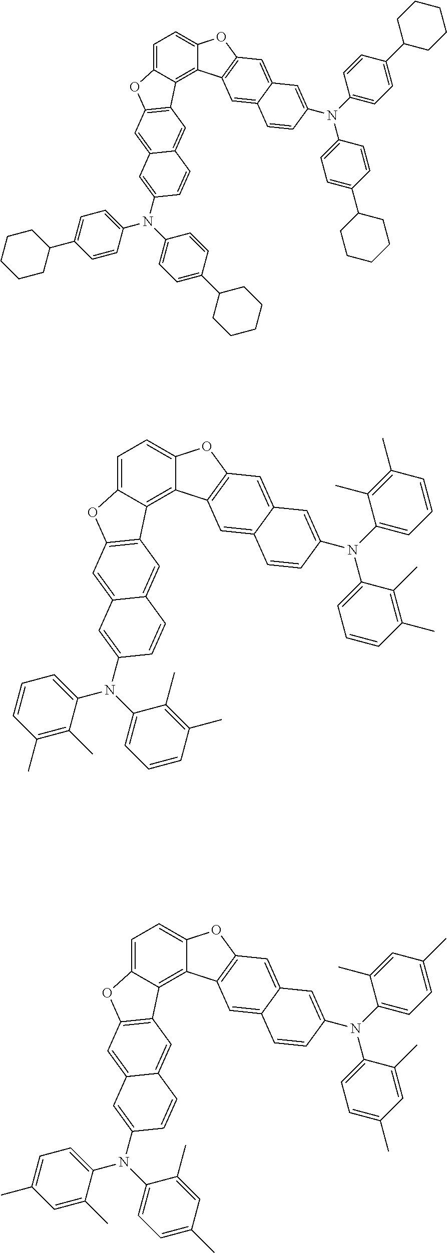

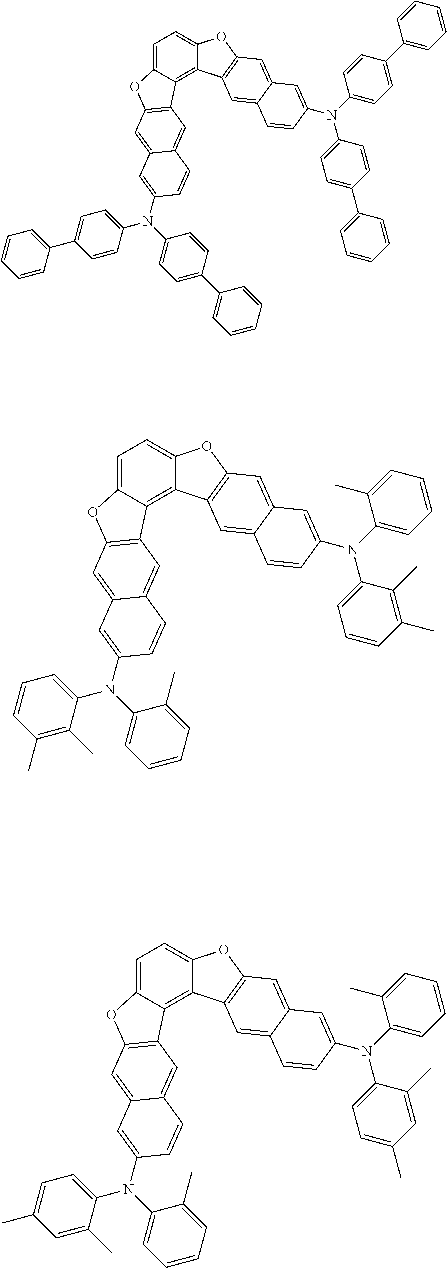

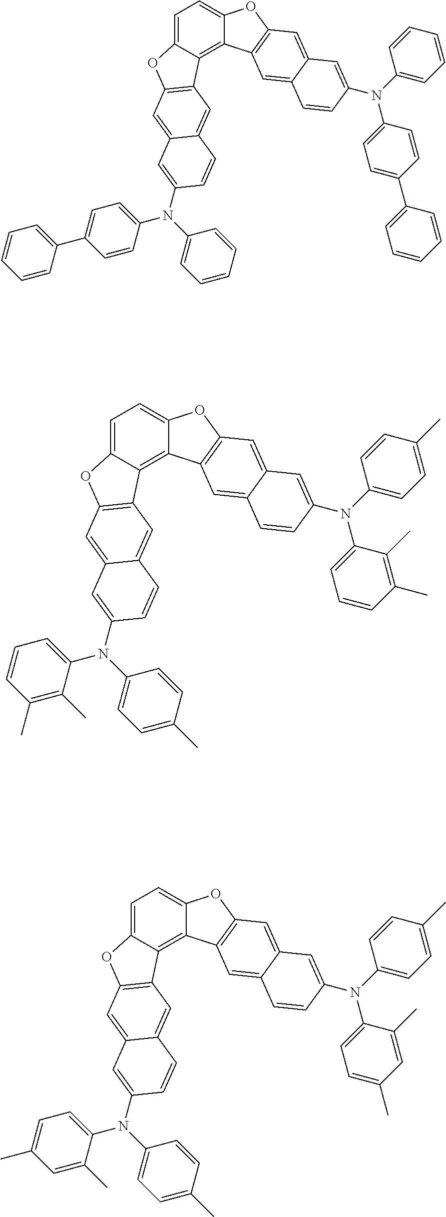

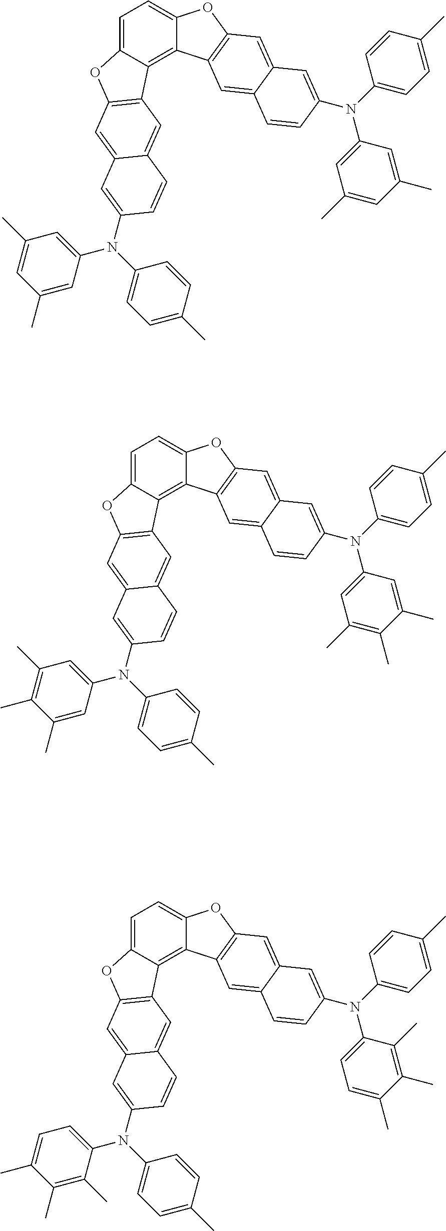

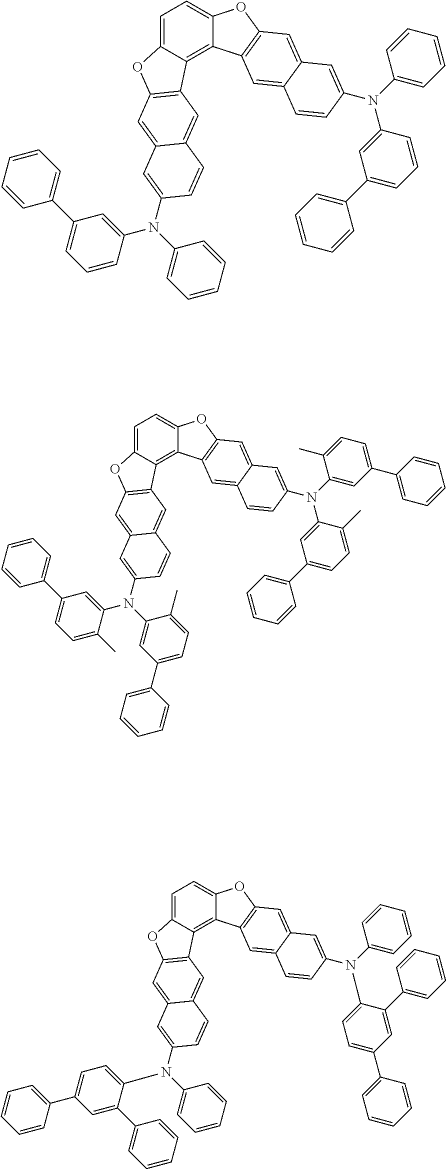 [SMILES: c1ccc(-c2cccc(N(c3ccccc3)c3ccc4cc5c(cc4c3)oc3ccc4oc6cc7cc(N(c8ccccc8)c8cccc(-c9ccccc9)c8)ccc7cc6c4c35)c2)cc1 ; Cc1ccc(-c2ccccc2)cc1N(c1ccc2cc3c(cc2c1)oc1ccc2oc4cc5cc(N(c6cc(-c7ccccc7)ccc6C)c6cc(-c7ccccc7)ccc6C)ccc5cc4c2c13)c1cc(-c2ccccc2)ccc1C ; c1ccc(-c2ccc(N(c3ccccc3)c3ccc4cc5c(cc4c3)oc3ccc4oc6cc7cc(N(c8ccccc8)c8ccc(-c9ccccc9)cc8-c8ccccc8)ccc7cc6c4c35)c(-c3ccccc3)c2)cc1]

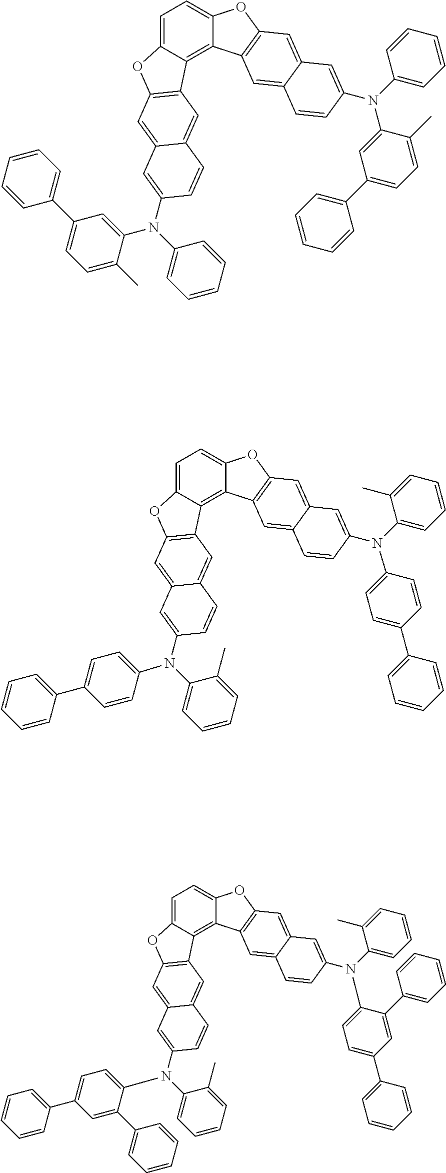 [SMILES: Cc1ccc(-c2ccccc2)cc1N(c1ccccc1)c1ccc2cc3c(cc2c1)oc1ccc2oc4cc5cc(N(c6ccccc6)c6cc(-c7ccccc7)ccc6C)ccc5cc4c2c13 ; Cc1ccccc1N(c1ccc(-c2ccccc2)cc1)c1ccc2cc3c(cc2c1)oc1ccc2oc4cc5cc(N(c6ccc(-c7ccccc7)cc6)c6ccccc6C)ccc5cc4c2c13 ; Cc1ccccc1N(c1ccc2cc3c(cc2c1)oc1ccc2oc4cc5cc(N(c6ccccc6C)c6ccc(-c7ccccc7)cc6-c6ccccc6)ccc5cc4c2c13)c1ccc(-c2ccccc2)cc1-c1ccccc1]

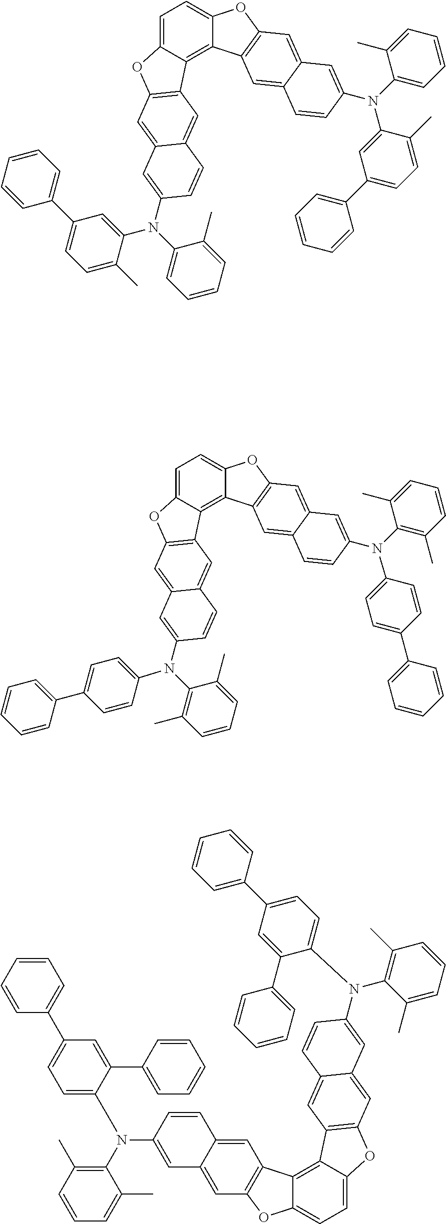 [SMILES: Cc1ccccc1N(c1ccc2cc3c(cc2c1)oc1ccc2oc4cc5cc(N(c6ccccc6C)c6cc(-c7ccccc7)ccc6C)ccc5cc4c2c13)c1cc(-c2ccccc2)ccc1C ; Cc1cccc(C)c1N(c1ccc(-c2ccccc2)cc1)c1ccc2cc3c(cc2c1)oc1ccc2oc4cc5cc(N(c6ccc(-c7ccccc7)cc6)c6c(C)cccc6C)ccc5cc4c2c13 ; Cc1cccc(C)c1N(c1ccc2cc3c(cc2c1)oc1ccc2oc4cc5cc(N(c6ccc(-c7ccccc7)cc6-c6ccccc6)c6c(C)cccc6C)ccc5cc4c2c13)c1ccc(-c2ccccc2)cc1-c1ccccc1]

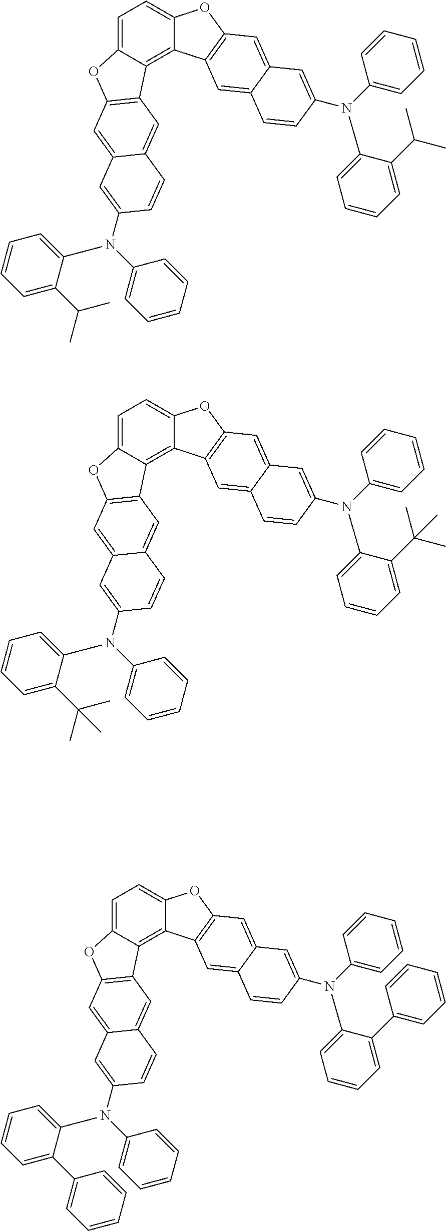 [SMILES: CC(C)c1ccccc1N(c1ccccc1)c1ccc2cc3c(cc2c1)oc1ccc2oc4cc5cc(N(c6ccccc6)c6ccccc6C(C)C)ccc5cc4c2c13 ; CC(C)(C)c1ccccc1N(c1ccccc1)c1ccc2cc3c(cc2c1)oc1ccc2oc4cc5cc(N(c6ccccc6)c6ccccc6C(C)(C)C)ccc5cc4c2c13 ; c1ccc(-c2ccccc2N(c2ccccc2)c2ccc3cc4c(cc3c2)oc2ccc3oc5cc6cc(N(c7ccccc7)c7ccccc7-c7ccccc7)ccc6cc5c3c24)cc1]

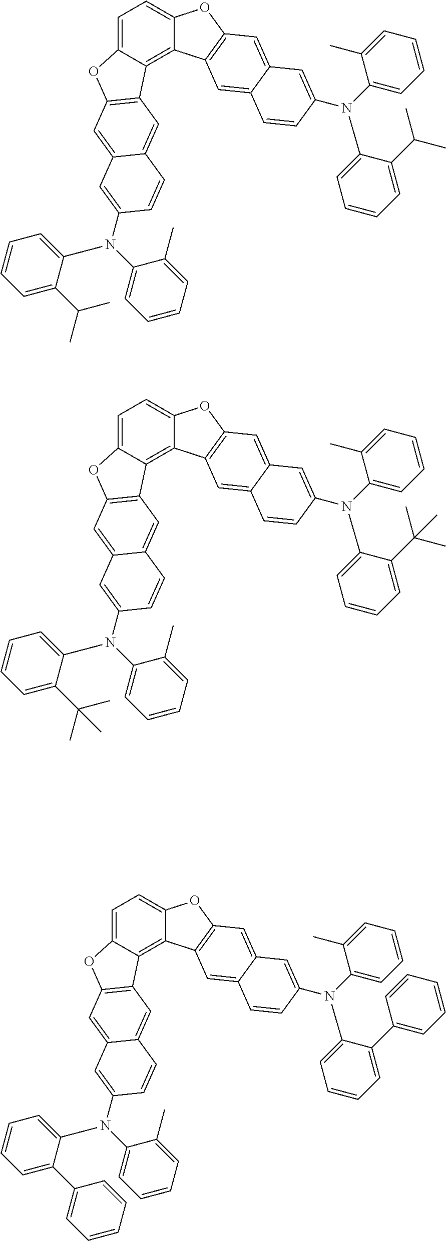 [SMILES: Cc1ccccc1N(c1ccc2cc3c(cc2c1)oc1ccc2oc4cc5cc(N(c6ccccc6C)c6ccccc6C(C)C)ccc5cc4c2c13)c1ccccc1C(C)C ; Cc1ccccc1N(c1ccc2cc3c(cc2c1)oc1ccc2oc4cc5cc(N(c6ccccc6C)c6ccccc6C(C)(C)C)ccc5cc4c2c13)c1ccccc1C(C)(C)C ; Cc1ccccc1N(c1ccc2cc3c(cc2c1)oc1ccc2oc4cc5cc(N(c6ccccc6C)c6ccccc6-c6ccccc6)ccc5cc4c2c13)c1ccccc1-c1ccccc1]

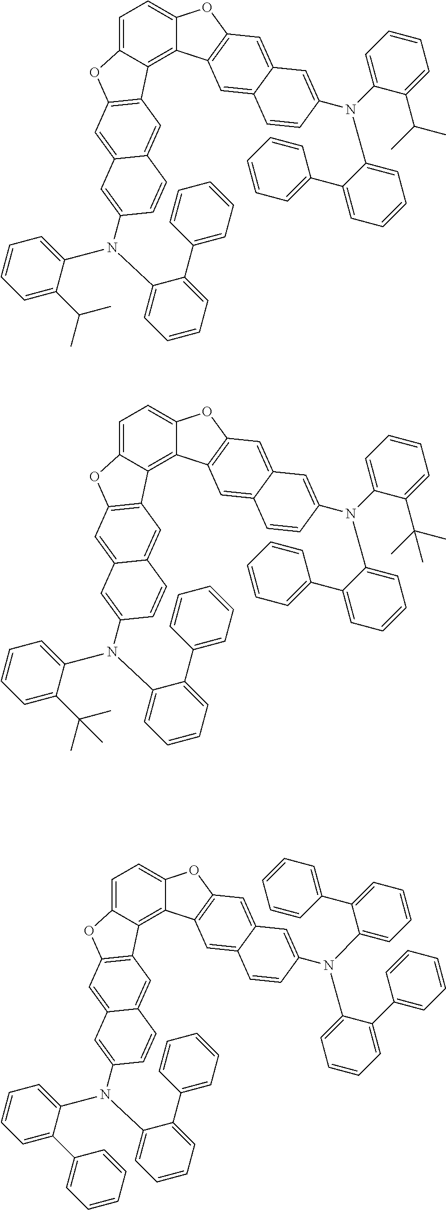 [SMILES: CC(C)c1ccccc1N(c1ccc2cc3c(cc2c1)oc1ccc2oc4cc5cc(N(c6ccccc6-c6ccccc6)c6ccccc6C(C)C)ccc5cc4c2c13)c1ccccc1-c1ccccc1 ; CC(C)(C)c1ccccc1N(c1ccc2cc3c(cc2c1)oc1ccc2oc4cc5cc(N(c6ccccc6-c6ccccc6)c6ccccc6C(C)(C)C)ccc5cc4c2c13)c1ccccc1-c1ccccc1 ; c1ccc(-c2ccccc2N(c2ccc3cc4c(cc3c2)oc2ccc3oc5cc6cc(N(c7ccccc7-c7ccccc7)c7ccccc7-c7ccccc7)ccc6cc5c3c24)c2ccccc2-c2ccccc2)cc1]

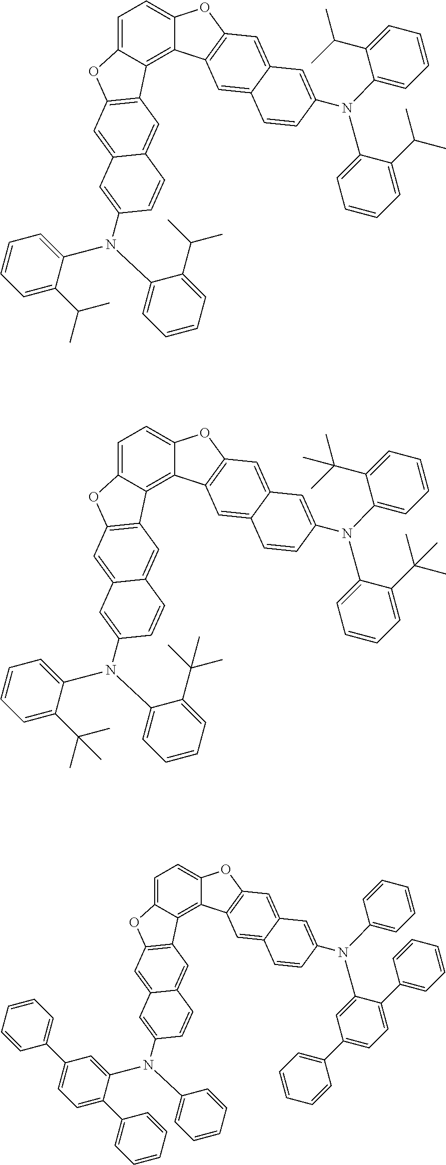 [SMILES: CC(C)c1ccccc1N(c1ccc2cc3c(cc2c1)oc1ccc2oc4cc5cc(N(c6ccccc6C(C)C)c6ccccc6C(C)C)ccc5cc4c2c13)c1ccccc1C(C)C ; CC(C)(C)c1ccccc1N(c1ccc2cc3c(cc2c1)oc1ccc2oc4cc5cc(N(c6ccccc6C(C)(C)C)c6ccccc6C(C)(C)C)ccc5cc4c2c13)c1ccccc1C(C)(C)C ; c1ccc(-c2ccc(-c3ccccc3)c(N(c3ccccc3)c3ccc4cc5c(cc4c3)oc3ccc4oc6cc7cc(N(c8ccccc8)c8cc(-c9ccccc9)ccc8-c8ccccc8)ccc7cc6c4c35)c2)cc1]

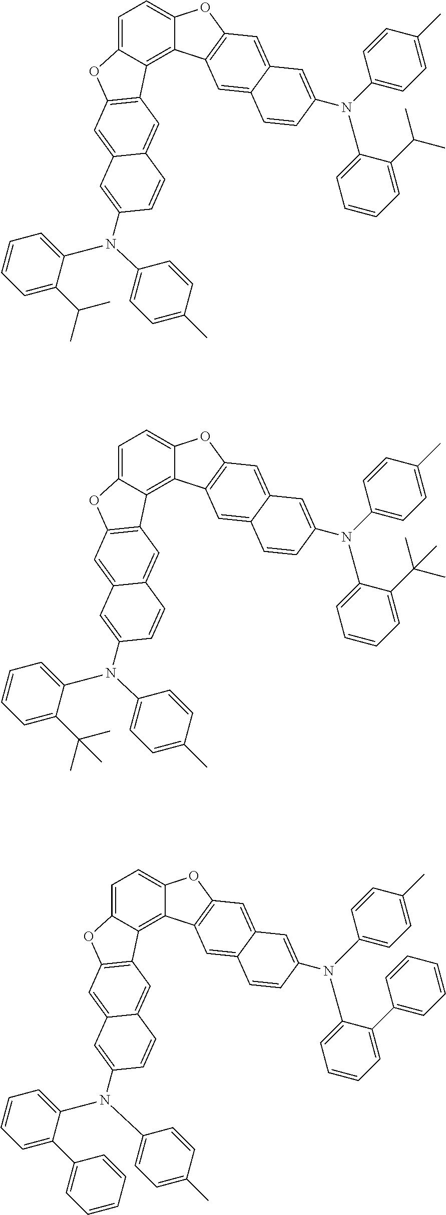 [SMILES: Cc1ccc(N(c2ccc3cc4c(cc3c2)oc2ccc3oc5cc6cc(N(c7ccc(C)cc7)c7ccccc7C(C)C)ccc6cc5c3c24)c2ccccc2C(C)C)cc1 ; Cc1ccc(N(c2ccc3cc4c(cc3c2)oc2ccc3oc5cc6cc(N(c7ccc(C)cc7)c7ccccc7C(C)(C)C)ccc6cc5c3c24)c2ccccc2C(C)(C)C)cc1 ; Cc1ccc(N(c2ccc3cc4c(cc3c2)oc2ccc3oc5cc6cc(N(c7ccc(C)cc7)c7ccccc7-c7ccccc7)ccc6cc5c3c24)c2ccccc2-c2ccccc2)cc1]

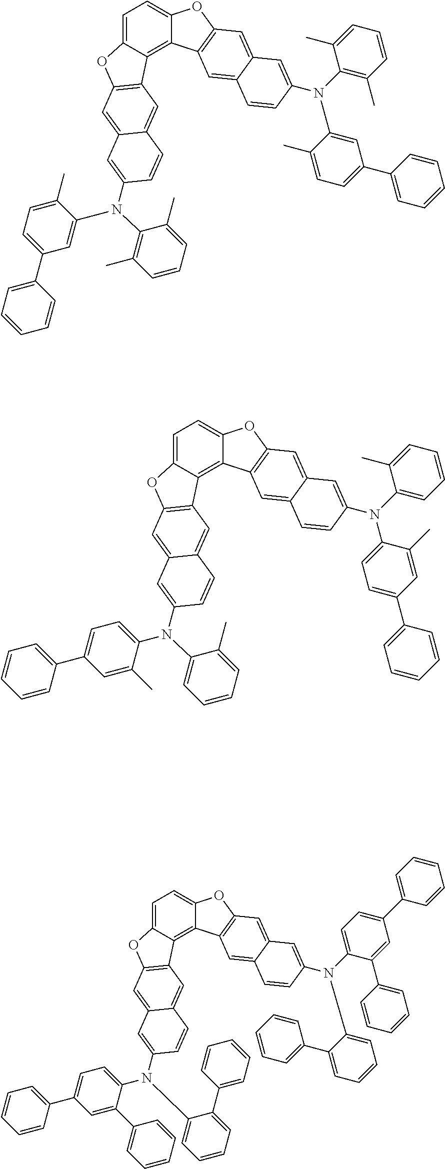 [SMILES: Cc1ccc(-c2ccccc2)cc1N(c1ccc2cc3c(cc2c1)oc1ccc2oc4cc5cc(N(c6cc(-c7ccccc7)ccc6C)c6c(C)cccc6C)ccc5cc4c2c13)c1c(C)cccc1C ; Cc1ccccc1N(c1ccc2cc3c(cc2c1)oc1ccc2oc4cc5cc(N(c6ccccc6C)c6ccc(-c7ccccc7)cc6C)ccc5cc4c2c13)c1ccc(-c2ccccc2)cc1C ; c1ccc(-c2ccc(N(c3ccc4cc5c(cc4c3)oc3ccc4oc6cc7cc(N(c8ccccc8-c8ccccc8)c8ccc(-c9ccccc9)cc8-c8ccccc8)ccc7cc6c4c35)c3ccccc3-c3ccccc3)c(-c3ccccc3)c2)cc1]

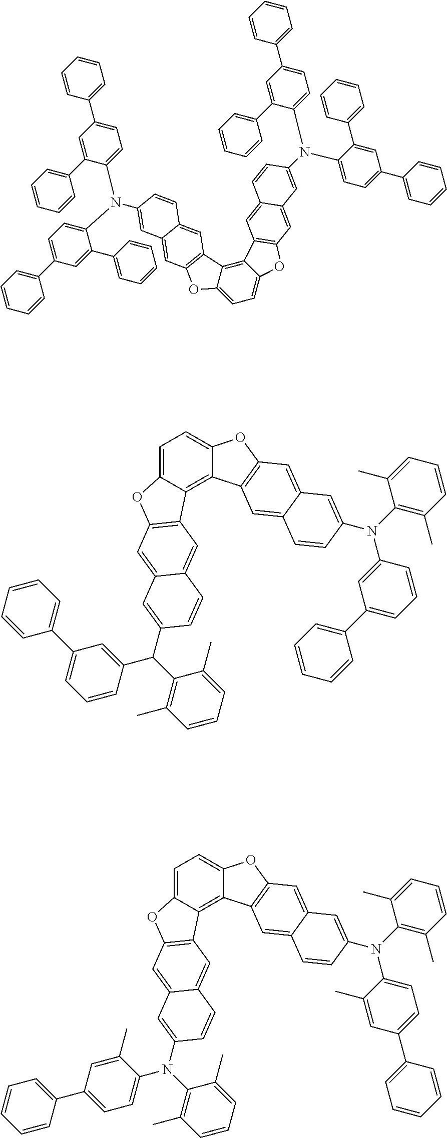 [SMILES: c1ccc(-c2ccc(N(c3ccc4cc5c(cc4c3)oc3ccc4oc6cc7cc(N(c8ccc(-c9ccccc9)cc8-c8ccccc8)c8ccc(-c9ccccc9)cc8-c8ccccc8)ccc7cc6c4c35)c3ccc(-c4ccccc4)cc3-c3ccccc3)c(-c3ccccc3)c2)cc1 ; Cc1cccc(C)c1C(c1cccc(-c2ccccc2)c1)c1ccc2cc3c(cc2c1)oc1ccc2oc4cc5cc(N(c6cccc(-c7ccccc7)c6)c6c(C)cccc6C)ccc5cc4c2c13 ; Cc1cc(-c2ccccc2)ccc1N(c1ccc2cc3c(cc2c1)oc1ccc2oc4cc5cc(N(c6ccc(-c7ccccc7)cc6C)c6c(C)cccc6C)ccc5cc4c2c13)c1c(C)cccc1C]

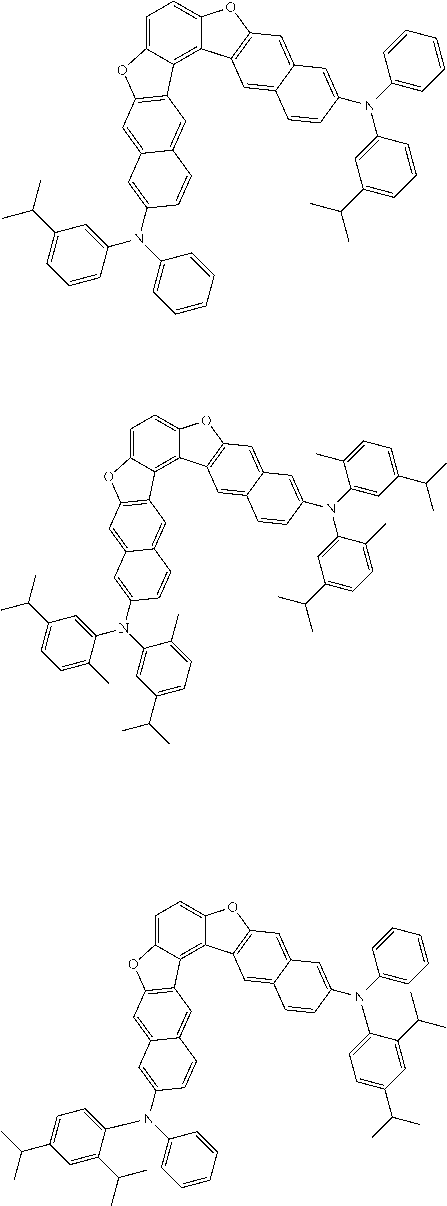

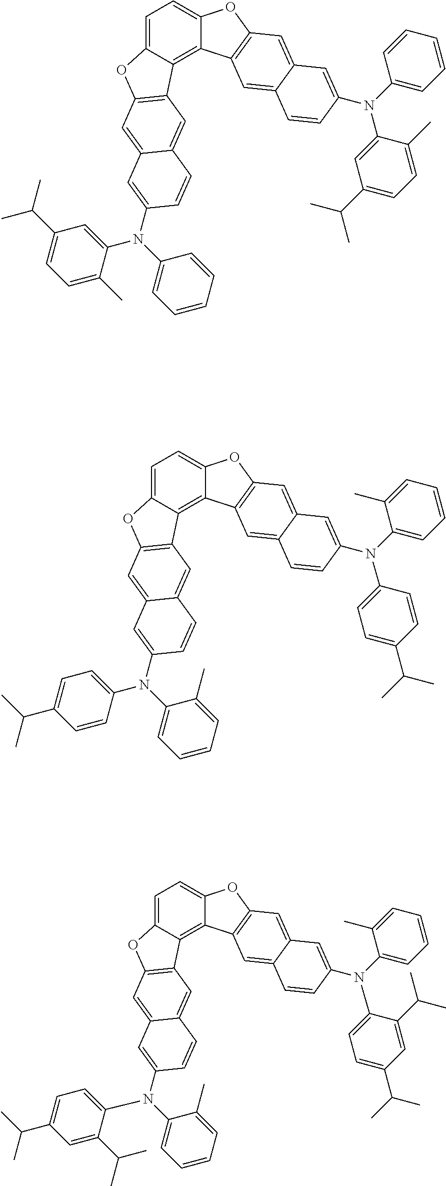 [SMILES: Cc1ccc(C(C)C)cc1N(c1ccccc1)c1ccc2cc3c(cc2c1)oc1ccc2oc4cc5cc(N(c6ccccc6)c6cc(C(C)C)ccc6C)ccc5cc4c2c13 ; Cc1ccccc1N(c1ccc(C(C)C)cc1)c1ccc2cc3c(cc2c1)oc1ccc2oc4cc5cc(N(c6ccc(C(C)C)cc6)c6ccccc6C)ccc5cc4c2c13 ; Cc1ccccc1N(c1ccc2cc3c(cc2c1)oc1ccc2oc4cc5cc(N(c6ccccc6C)c6ccc(C(C)C)cc6C(C)C)ccc5cc4c2c13)c1ccc(C(C)C)cc1C(C)C]

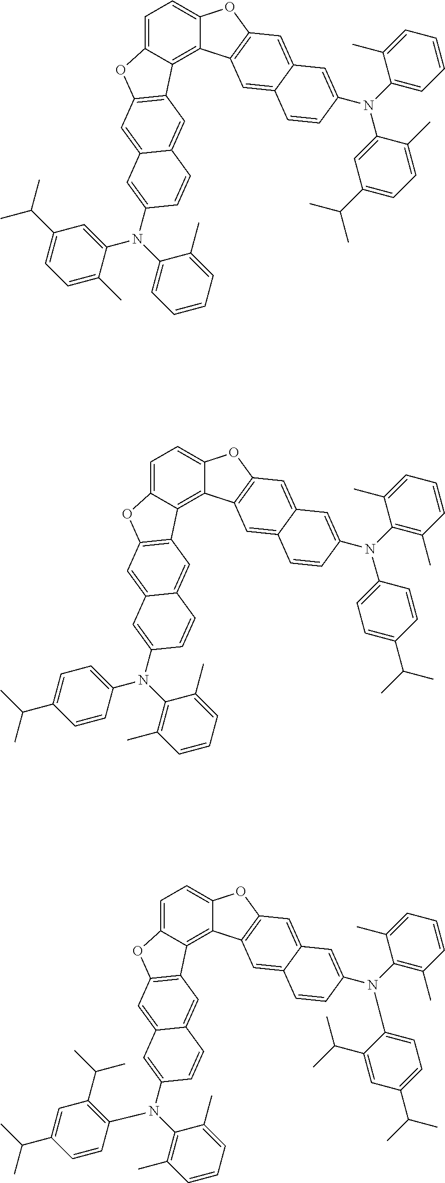 [SMILES: Cc1ccccc1N(c1ccc2cc3c(cc2c1)oc1ccc2oc4cc5cc(N(c6ccccc6C)c6cc(C(C)C)ccc6C)ccc5cc4c2c13)c1cc(C(C)C)ccc1C ; Cc1cccc(C)c1N(c1ccc(C(C)C)cc1)c1ccc2cc3c(cc2c1)oc1ccc2oc4cc5cc(N(c6ccc(C(C)C)cc6)c6c(C)cccc6C)ccc5cc4c2c13 ; Cc1cccc(C)c1N(c1ccc2cc3c(cc2c1)oc1ccc2oc4cc5cc(N(c6ccc(C(C)C)cc6C(C)C)c6c(C)cccc6C)ccc5cc4c2c13)c1ccc(C(C)C)cc1C(C)C]

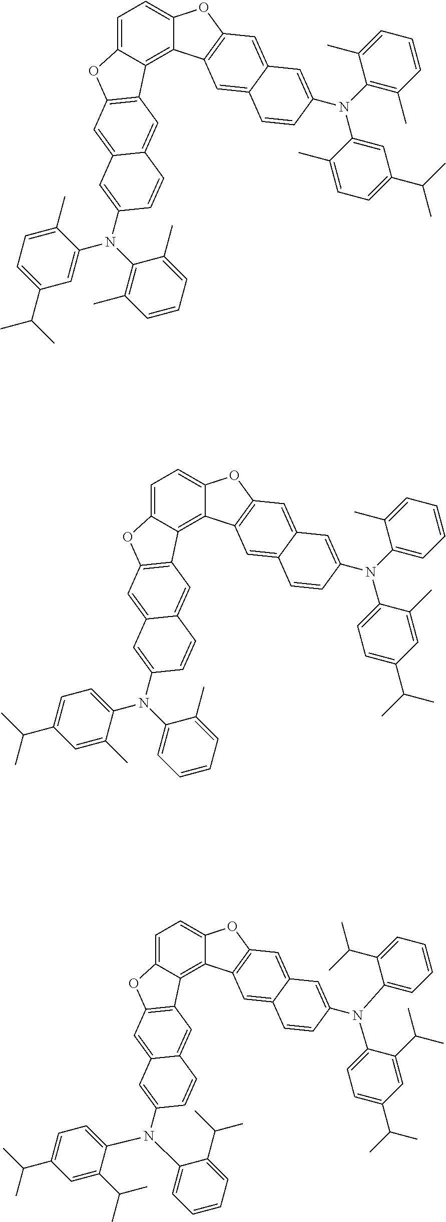

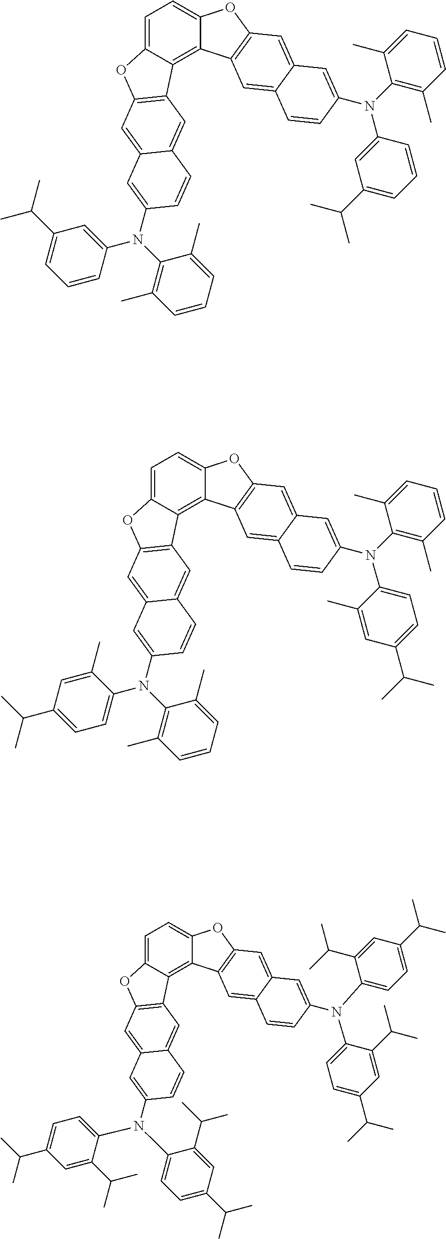 [SMILES: Cc1cccc(C)c1N(c1cccc(C(C)C)c1)c1ccc2cc3c(cc2c1)oc1ccc2oc4cc5cc(N(c6cccc(C(C)C)c6)c6c(C)cccc6C)ccc5cc4c2c13 ; Cc1cc(C(C)C)ccc1N(c1ccc2cc3c(cc2c1)oc1ccc2oc4cc5cc(N(c6ccc(C(C)C)cc6C)c6c(C)cccc6C)ccc5cc4c2c13)c1c(C)cccc1C ; CC(C)c1ccc(N(c2ccc3cc4c(cc3c2)oc2ccc3oc5cc6cc(N(c7ccc(C(C)C)cc7C(C)C)c7ccc(C(C)C)cc7C(C)C)ccc6cc5c3c24)c2ccc(C(C)C)cc2C(C)C)c(C(C)C)c1]

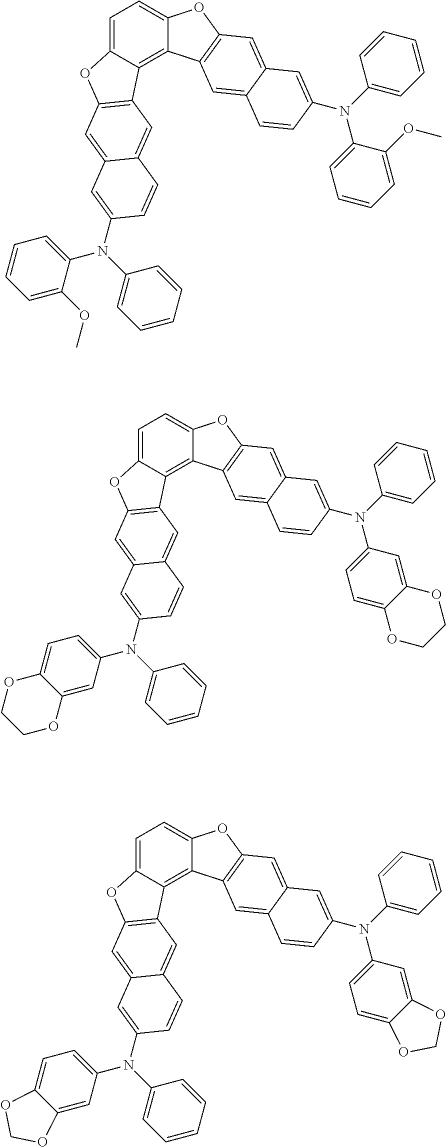 [SMILES: COc1ccccc1N(c1ccccc1)c1ccc2cc3c(cc2c1)oc1ccc2oc4cc5cc(N(c6ccccc6)c6ccccc6OC)ccc5cc4c2c13 ; c1ccc(N(c2ccc3c(c2)OCCO3)c2ccc3cc4c(cc3c2)oc2ccc3oc5cc6cc(N(c7ccccc7)c7ccc8c(c7)OCCO8)ccc6cc5c3c24)cc1 ; c1ccc(N(c2ccc3c(c2)OCO3)c2ccc3cc4c(cc3c2)oc2ccc3oc5cc6cc(N(c7ccccc7)c7ccc8c(c7)OCO8)ccc6cc5c3c24)cc1]

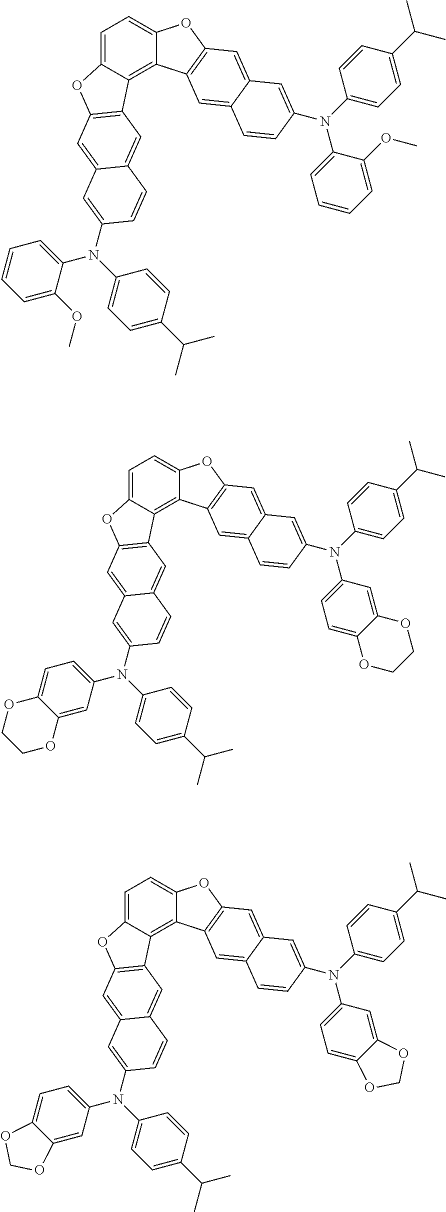 [SMILES: COc1ccccc1N(c1ccc(C(C)C)cc1)c1ccc2cc3c(cc2c1)oc1ccc2oc4cc5cc(N(c6ccc(C(C)C)cc6)c6ccccc6OC)ccc5cc4c2c13 ; CC(C)c1ccc(N(c2ccc3c(c2)OCCO3)c2ccc3cc4c(cc3c2)oc2ccc3oc5cc6cc(N(c7ccc(C(C)C)cc7)c7ccc8c(c7)OCCO8)ccc6cc5c3c24)cc1 ; CC(C)c1ccc(N(c2ccc3c(c2)OCO3)c2ccc3cc4c(cc3c2)oc2ccc3oc5cc6cc(N(c7ccc(C(C)C)cc7)c7ccc8c(c7)OCO8)ccc6cc5c3c24)cc1]

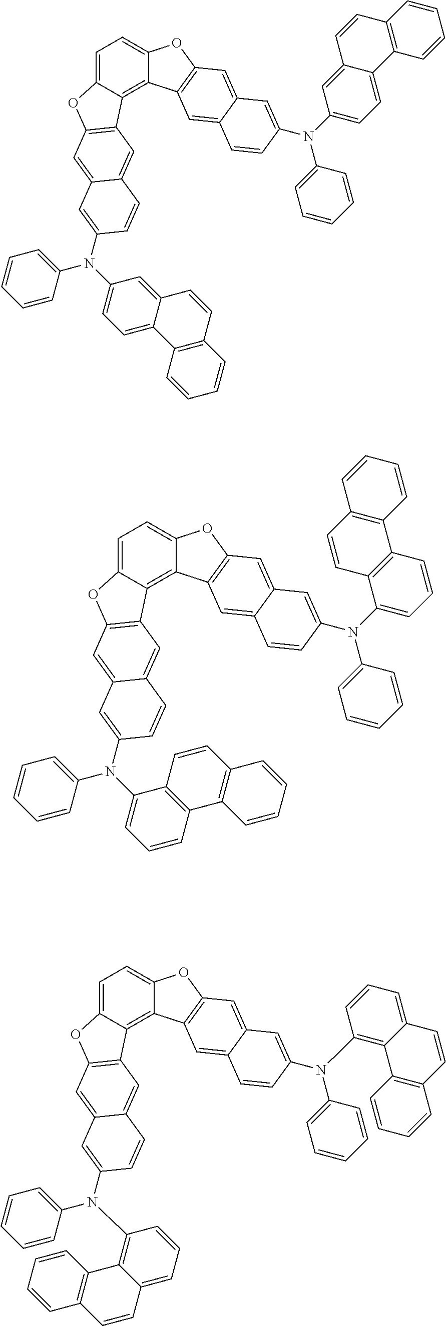 [SMILES: c1ccc(N(c2ccc3cc4c(cc3c2)oc2ccc3oc5cc6cc(N(c7ccccc7)c7ccc8c(ccc9ccccc98)c7)ccc6cc5c3c24)c2ccc3c(ccc4ccccc43)c2)cc1 ; c1ccc(N(c2ccc3cc4c(cc3c2)oc2ccc3oc5cc6cc(N(c7ccccc7)c7cccc8c7ccc7ccccc78)ccc6cc5c3c24)c2cccc3c2ccc2ccccc23)cc1 ; c1ccc(N(c2ccc3cc4c(cc3c2)oc2ccc3oc5cc6cc(N(c7ccccc7)c7cccc8ccc9ccccc9c78)ccc6cc5c3c24)c2cccc3ccc4ccccc4c23)cc1]

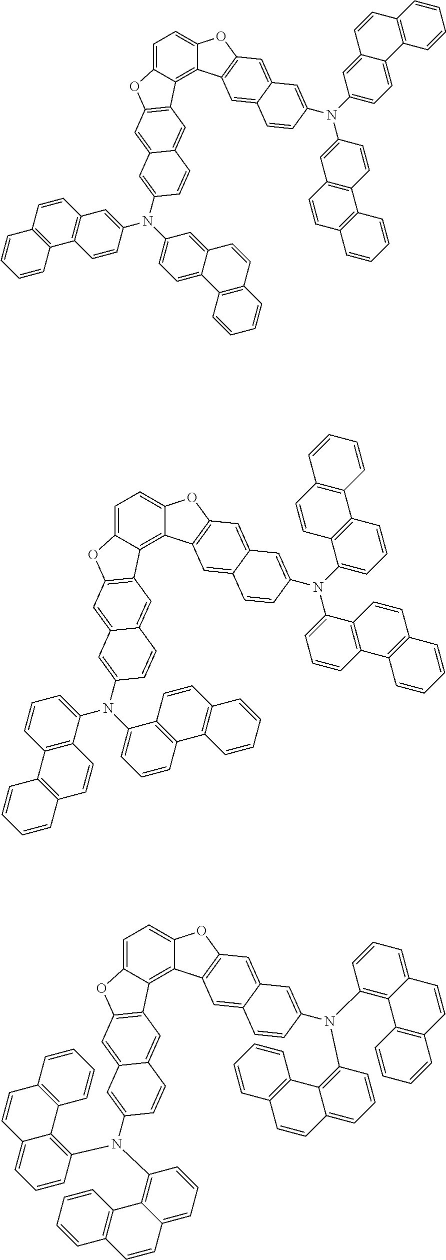 [SMILES: c1ccc2c(c1)ccc1cc(N(c3ccc4cc5c(cc4c3)oc3ccc4oc6cc7cc(N(c8ccc9c(ccc%10ccccc%109)c8)c8ccc9c(ccc%10ccccc%109)c8)ccc7cc6c4c35)c3ccc4c(ccc5ccccc54)c3)ccc12 ; c1ccc2c(c1)ccc1c(N(c3ccc4cc5c(cc4c3)oc3ccc4oc6cc7cc(N(c8cccc9c8ccc8ccccc89)c8cccc9c8ccc8ccccc89)ccc7cc6c4c35)c3cccc4c3ccc3ccccc34)cccc12 ; c1ccc2c(c1)ccc1cccc(N(c3ccc4cc5c(cc4c3)oc3ccc4oc6cc7cc(N(c8cccc9ccc%10ccccc%10c89)c8cccc9ccc%10ccccc%10c89)ccc7cc6c4c35)c3cccc4ccc5ccccc5c34)c12]

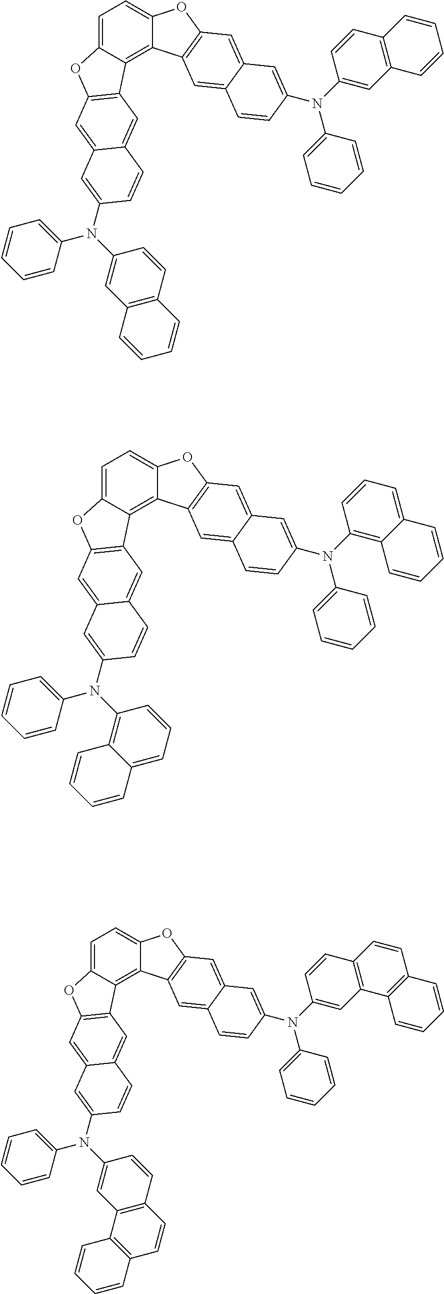 [SMILES: c1ccc(N(c2ccc3ccccc3c2)c2ccc3cc4c(cc3c2)oc2ccc3oc5cc6cc(N(c7ccccc7)c7ccc8ccccc8c7)ccc6cc5c3c24)cc1 ; c1ccc(N(c2ccc3cc4c(cc3c2)oc2ccc3oc5cc6cc(N(c7ccccc7)c7cccc8ccccc78)ccc6cc5c3c24)c2cccc3ccccc23)cc1 ; c1ccc(N(c2ccc3cc4c(cc3c2)oc2ccc3oc5cc6cc(N(c7ccccc7)c7ccc8ccc9ccccc9c8c7)ccc6cc5c3c24)c2ccc3ccc4ccccc4c3c2)cc1]

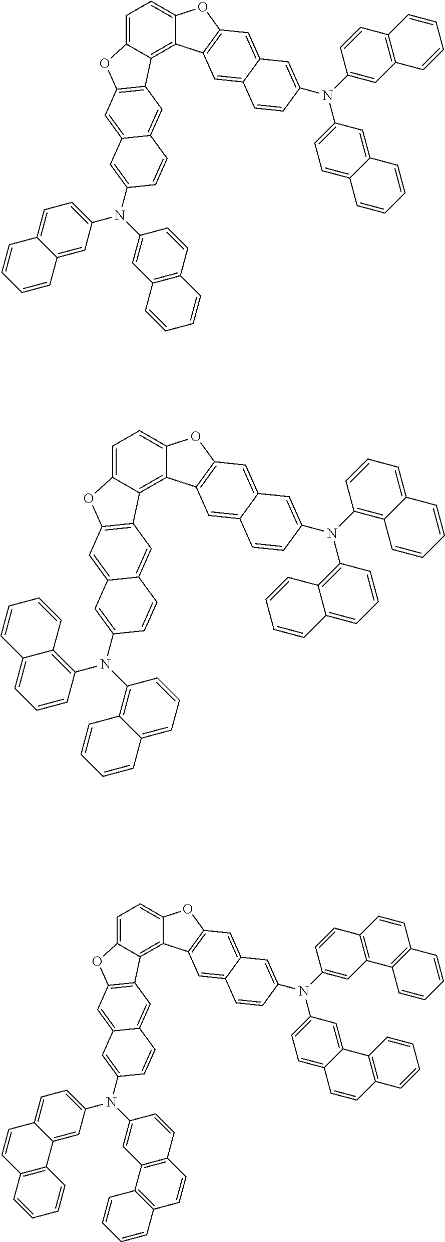 [SMILES: c1ccc2cc(N(c3ccc4ccccc4c3)c3ccc4cc5c(cc4c3)oc3ccc4oc6cc7cc(N(c8ccc9ccccc9c8)c8ccc9ccccc9c8)ccc7cc6c4c35)ccc2c1 ; c1ccc2c(N(c3ccc4cc5c(cc4c3)oc3ccc4oc6cc7cc(N(c8cccc9ccccc89)c8cccc9ccccc89)ccc7cc6c4c35)c3cccc4ccccc34)cccc2c1 ; c1ccc2c(c1)ccc1ccc(N(c3ccc4cc5c(cc4c3)oc3ccc4oc6cc7cc(N(c8ccc9ccc%10ccccc%10c9c8)c8ccc9ccc%10ccccc%10c9c8)ccc7cc6c4c35)c3ccc4ccc5ccccc5c4c3)cc12]

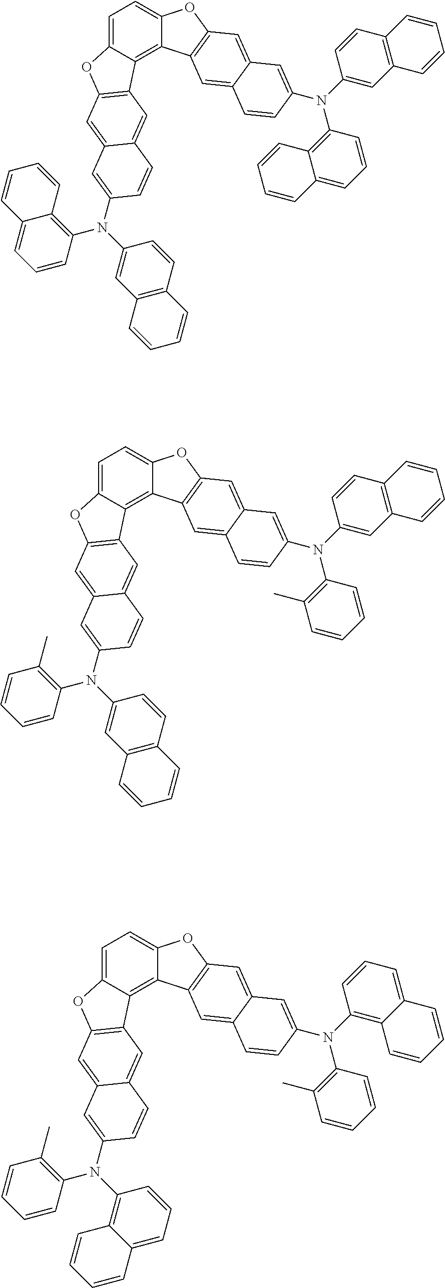 [SMILES: c1ccc2cc(N(c3ccc4cc5c(cc4c3)oc3ccc4oc6cc7cc(N(c8ccc9ccccc9c8)c8cccc9ccccc89)ccc7cc6c4c35)c3cccc4ccccc34)ccc2c1 ; Cc1ccccc1N(c1ccc2ccccc2c1)c1ccc2cc3c(cc2c1)oc1ccc2oc4cc5cc(N(c6ccc7ccccc7c6)c6ccccc6C)ccc5cc4c2c13 ; Cc1ccccc1N(c1ccc2cc3c(cc2c1)oc1ccc2oc4cc5cc(N(c6ccccc6C)c6cccc7ccccc67)ccc5cc4c2c13)c1cccc2ccccc12]

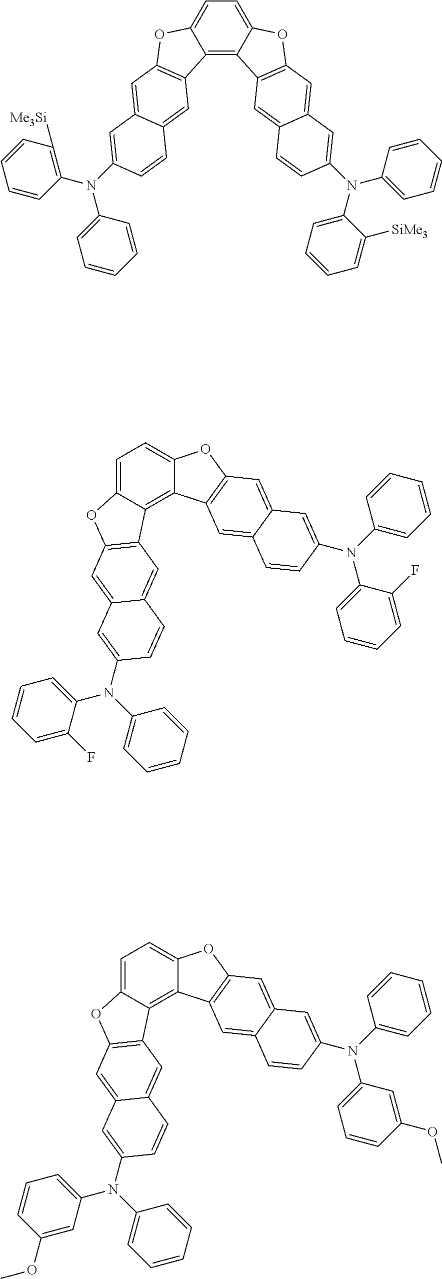

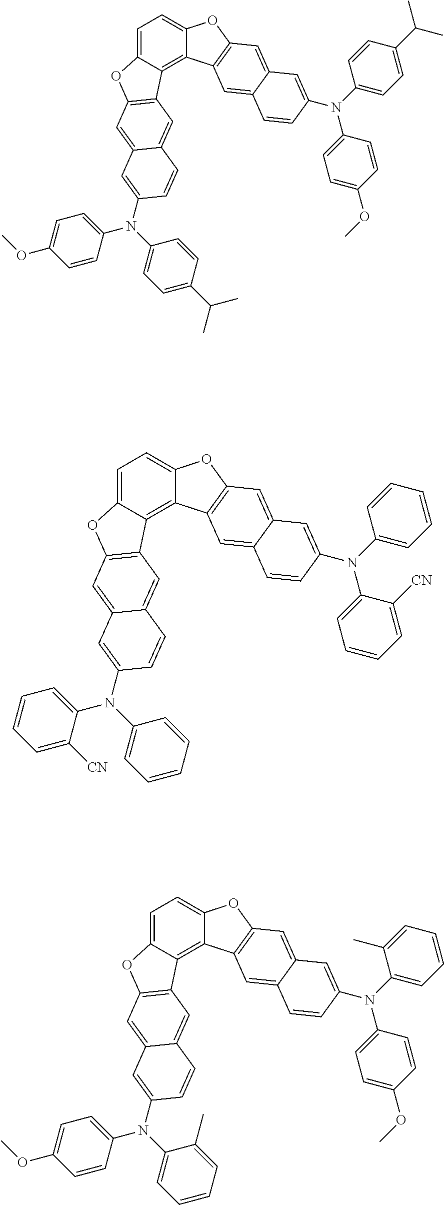

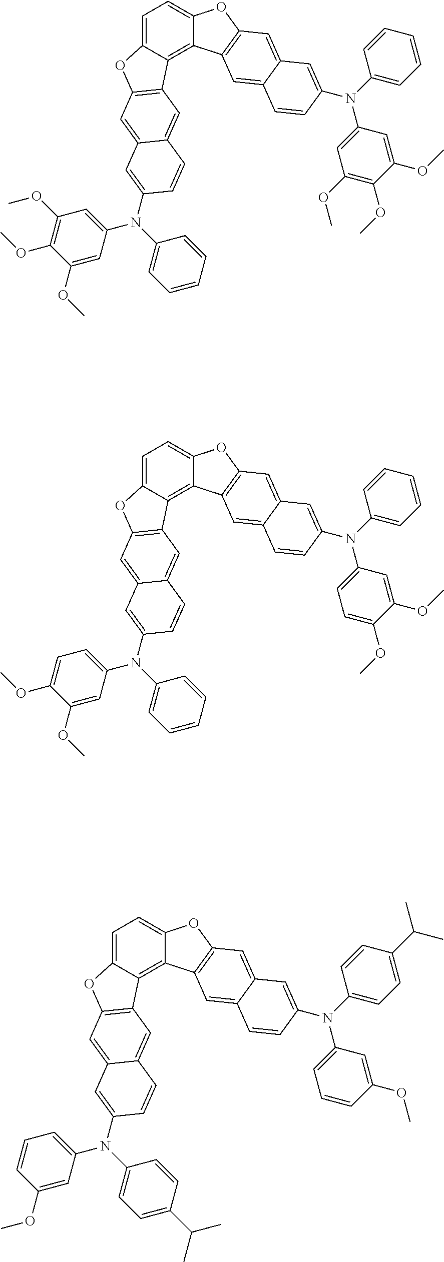

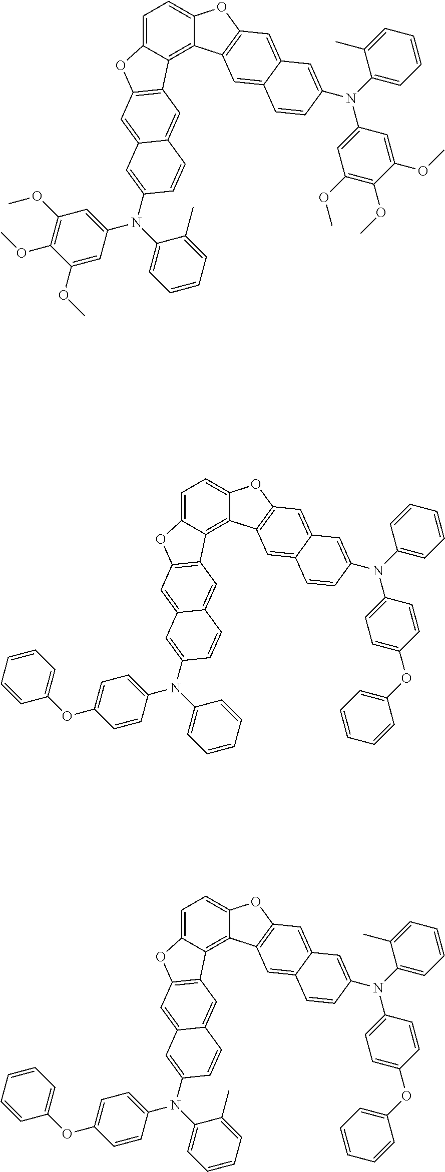

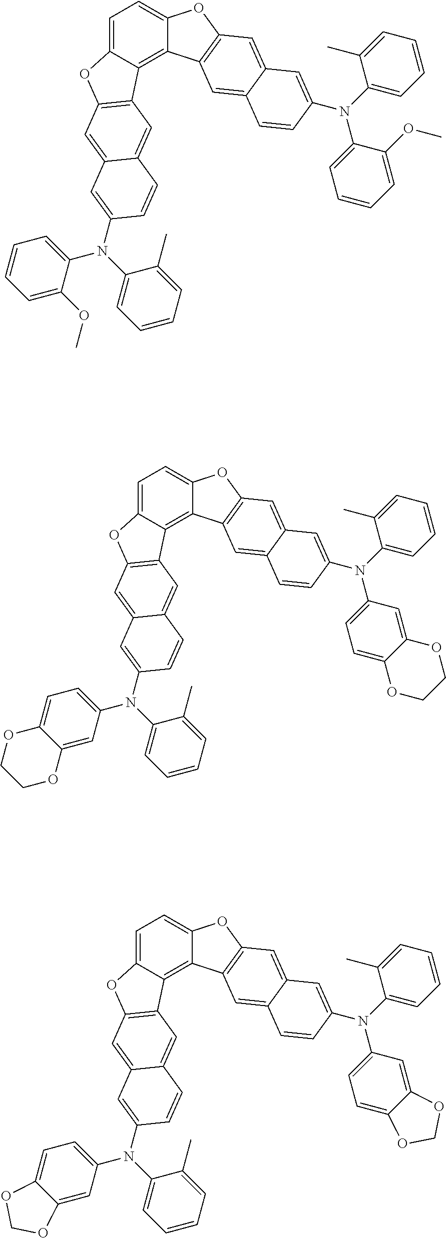 [SMILES: COc1ccccc1N(c1ccc2cc3c(cc2c1)oc1ccc2oc4cc5cc(N(c6ccccc6C)c6ccccc6OC)ccc5cc4c2c13)c1ccccc1C ; Cc1ccccc1N(c1ccc2c(c1)OCCO2)c1ccc2cc3c(cc2c1)oc1ccc2oc4cc5cc(N(c6ccc7c(c6)OCCO7)c6ccccc6C)ccc5cc4c2c13 ; Cc1ccccc1N(c1ccc2c(c1)OCO2)c1ccc2cc3c(cc2c1)oc1ccc2oc4cc5cc(N(c6ccc7c(c6)OCO7)c6ccccc6C)ccc5cc4c2c13]

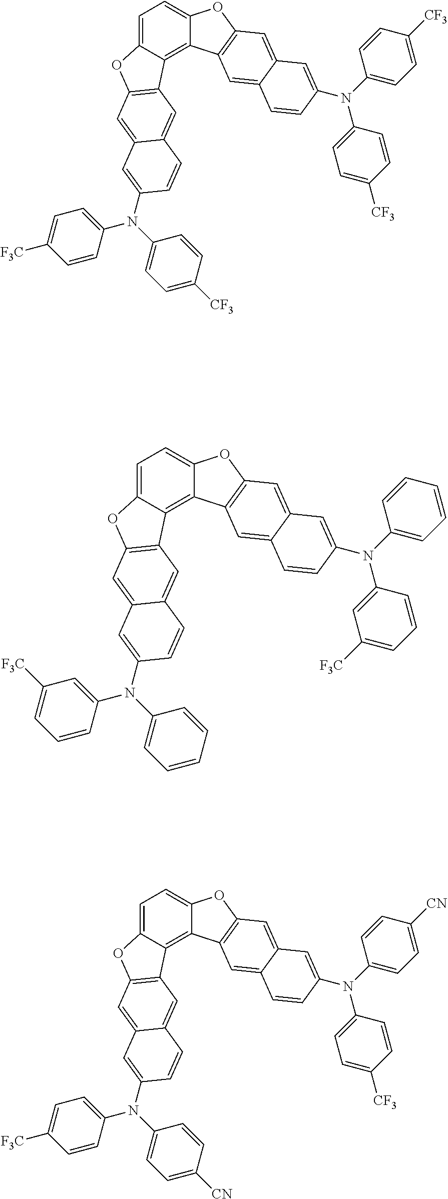 [SMILES: FC(F)(F)c1ccc(N(c2ccc(C(F)(F)F)cc2)c2ccc3cc4c(cc3c2)oc2ccc3oc5cc6cc(N(c7ccc(C(F)(F)F)cc7)c7ccc(C(F)(F)F)cc7)ccc6cc5c3c24)cc1 ; FC(F)(F)c1cccc(N(c2ccccc2)c2ccc3cc4c(cc3c2)oc2ccc3oc5cc6cc(N(c7ccccc7)c7cccc(C(F)(F)F)c7)ccc6cc5c3c24)c1 ; N#Cc1ccc(N(c2ccc(C(F)(F)F)cc2)c2ccc3cc4c(cc3c2)oc2ccc3oc5cc6cc(N(c7ccc(C#N)cc7)c7ccc(C(F)(F)F)cc7)ccc6cc5c3c24)cc1]

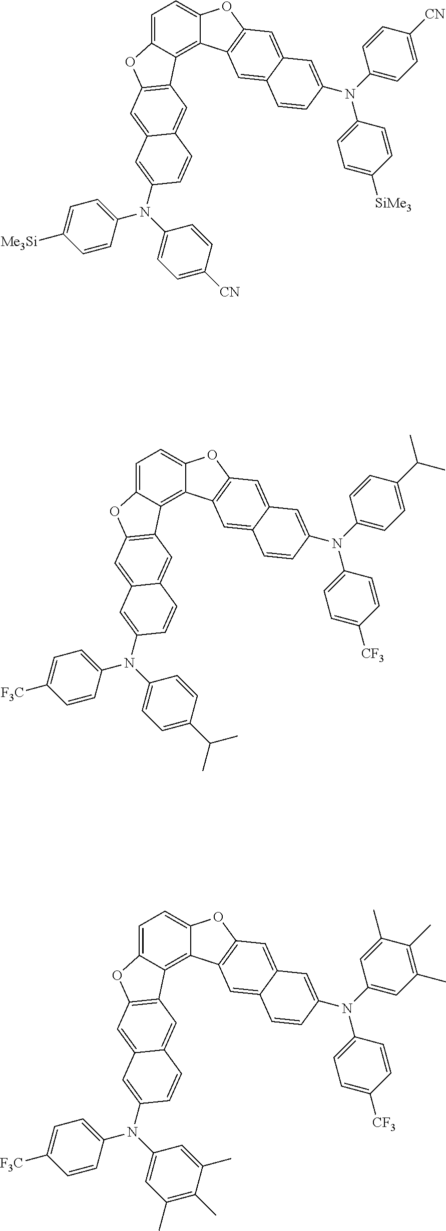 [SMILES: C[Si](C)(C)c1ccc(N(c2ccc(C#N)cc2)c2ccc3cc4c(cc3c2)oc2ccc3oc5cc6cc(N(c7ccc(C#N)cc7)c7ccc([Si](C)(C)C)cc7)ccc6cc5c3c24)cc1 ; CC(C)c1ccc(N(c2ccc(C(F)(F)F)cc2)c2ccc3cc4c(cc3c2)oc2ccc3oc5cc6cc(N(c7ccc(C(C)C)cc7)c7ccc(C(F)(F)F)cc7)ccc6cc5c3c24)cc1 ; Cc1cc(N(c2ccc(C(F)(F)F)cc2)c2ccc3cc4c(cc3c2)oc2ccc3oc5cc6cc(N(c7ccc(C(F)(F)F)cc7)c7cc(C)c(C)c(C)c7)ccc6cc5c3c24)cc(C)c1C]

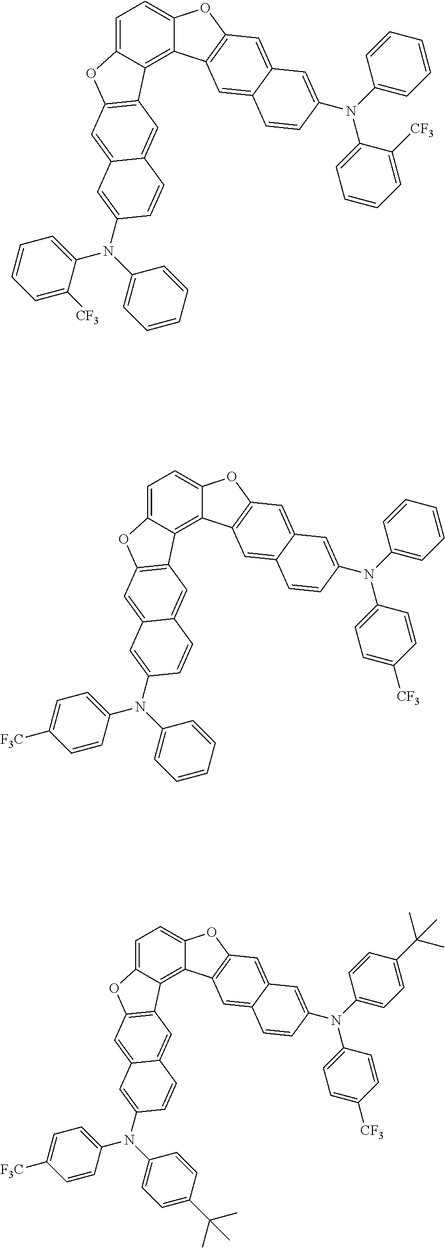 [SMILES: FC(F)(F)c1ccccc1N(c1ccccc1)c1ccc2cc3c(cc2c1)oc1ccc2oc4cc5cc(N(c6ccccc6)c6ccccc6C(F)(F)F)ccc5cc4c2c13 ; FC(F)(F)c1ccc(N(c2ccccc2)c2ccc3cc4c(cc3c2)oc2ccc3oc5cc6cc(N(c7ccccc7)c7ccc(C(F)(F)F)cc7)ccc6cc5c3c24)cc1 ; CC(C)(C)c1ccc(N(c2ccc(C(F)(F)F)cc2)c2ccc3cc4c(cc3c2)oc2ccc3oc5cc6cc(N(c7ccc(C(C)(C)C)cc7)c7ccc(C(F)(F)F)cc7)ccc6cc5c3c24)cc1]

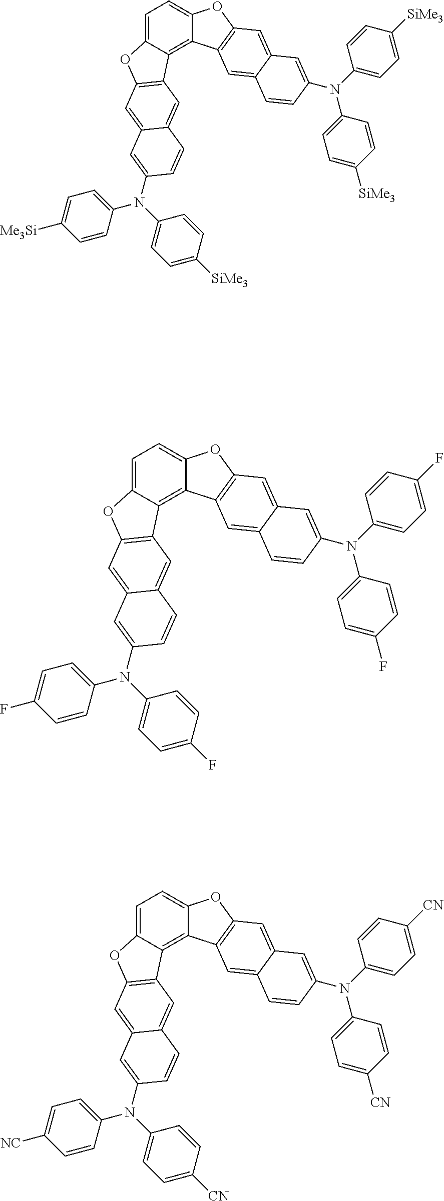 [SMILES: C[Si](C)(C)c1ccc(N(c2ccc([Si](C)(C)C)cc2)c2ccc3cc4c(cc3c2)oc2ccc3oc5cc6cc(N(c7ccc([Si](C)(C)C)cc7)c7ccc([Si](C)(C)C)cc7)ccc6cc5c3c24)cc1 ; Fc1ccc(N(c2ccc(F)cc2)c2ccc3cc4c(cc3c2)oc2ccc3oc5cc6cc(N(c7ccc(F)cc7)c7ccc(F)cc7)ccc6cc5c3c24)cc1 ; N#Cc1ccc(N(c2ccc(C#N)cc2)c2ccc3cc4c(cc3c2)oc2ccc3oc5cc6cc(N(c7ccc(C#N)cc7)c7ccc(C#N)cc7)ccc6cc5c3c24)cc1]

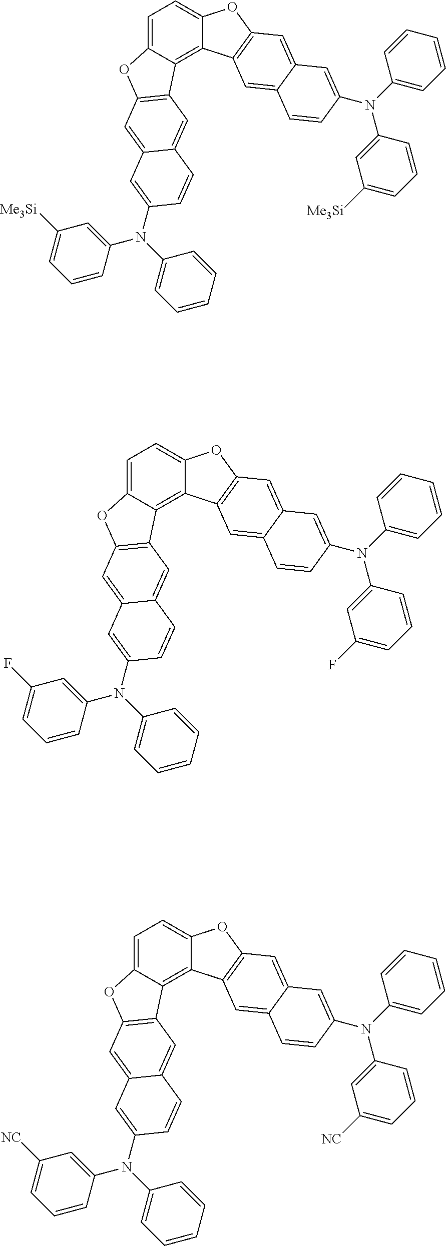 [SMILES: C[Si](C)(C)c1cccc(N(c2ccccc2)c2ccc3cc4c(cc3c2)oc2ccc3oc5cc6cc(N(c7ccccc7)c7cccc([Si](C)(C)C)c7)ccc6cc5c3c24)c1 ; Fc1cccc(N(c2ccccc2)c2ccc3cc4c(cc3c2)oc2ccc3oc5cc6cc(N(c7ccccc7)c7cccc(F)c7)ccc6cc5c3c24)c1 ; N#Cc1cccc(N(c2ccccc2)c2ccc3cc4c(cc3c2)oc2ccc3oc5cc6cc(N(c7ccccc7)c7cccc(C#N)c7)ccc6cc5c3c24)c1]

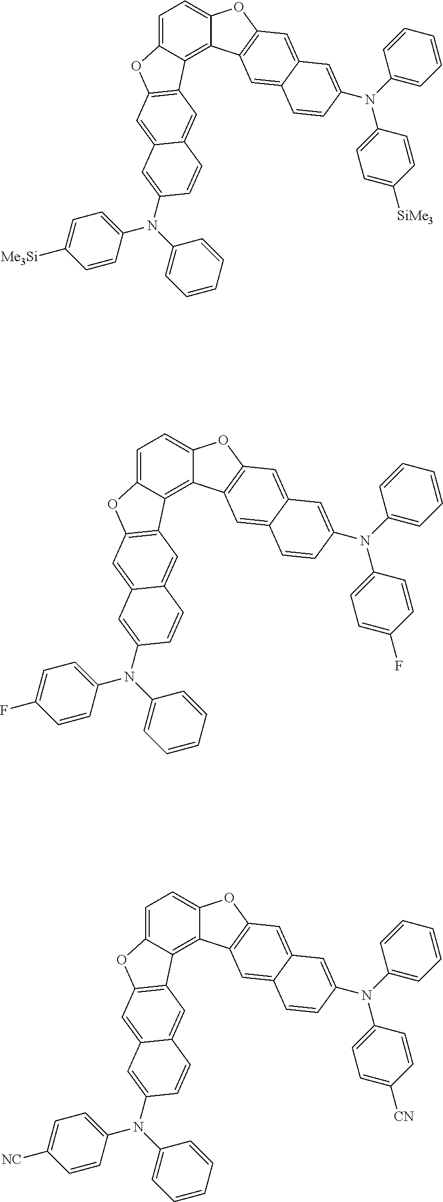 [SMILES: C[Si](C)(C)c1ccc(N(c2ccccc2)c2ccc3cc4c(cc3c2)oc2ccc3oc5cc6cc(N(c7ccccc7)c7ccc([Si](C)(C)C)cc7)ccc6cc5c3c24)cc1 ; Fc1ccc(N(c2ccccc2)c2ccc3cc4c(cc3c2)oc2ccc3oc5cc6cc(N(c7ccccc7)c7ccc(F)cc7)ccc6cc5c3c24)cc1 ; N#Cc1ccc(N(c2ccccc2)c2ccc3cc4c(cc3c2)oc2ccc3oc5cc6cc(N(c7ccccc7)c7ccc(C#N)cc7)ccc6cc5c3c24)cc1]

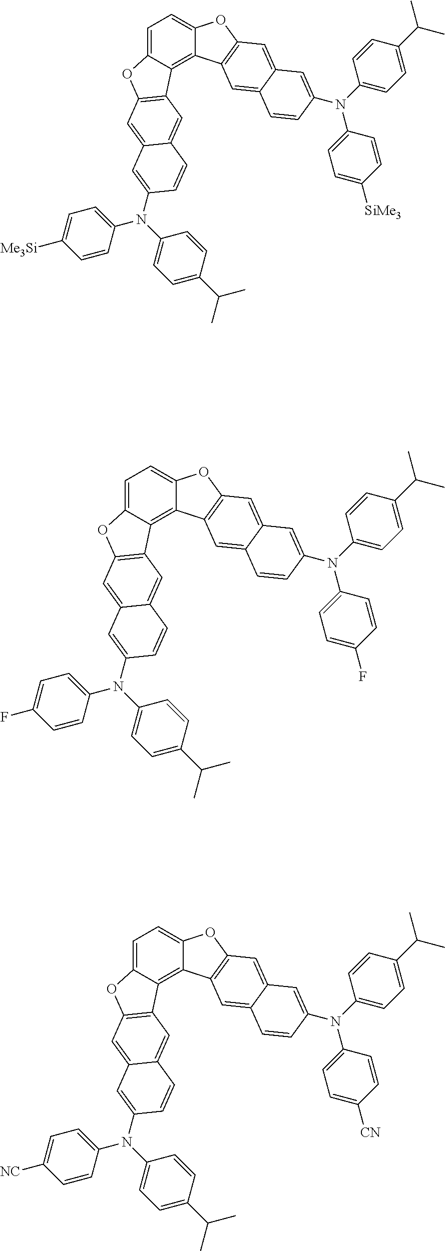 [SMILES: CC(C)c1ccc(N(c2ccc([Si](C)(C)C)cc2)c2ccc3cc4c(cc3c2)oc2ccc3oc5cc6cc(N(c7ccc(C(C)C)cc7)c7ccc([Si](C)(C)C)cc7)ccc6cc5c3c24)cc1 ; CC(C)c1ccc(N(c2ccc(F)cc2)c2ccc3cc4c(cc3c2)oc2ccc3oc5cc6cc(N(c7ccc(F)cc7)c7ccc(C(C)C)cc7)ccc6cc5c3c24)cc1 ; CC(C)c1ccc(N(c2ccc(C#N)cc2)c2ccc3cc4c(cc3c2)oc2ccc3oc5cc6cc(N(c7ccc(C#N)cc7)c7ccc(C(C)C)cc7)ccc6cc5c3c24)cc1]

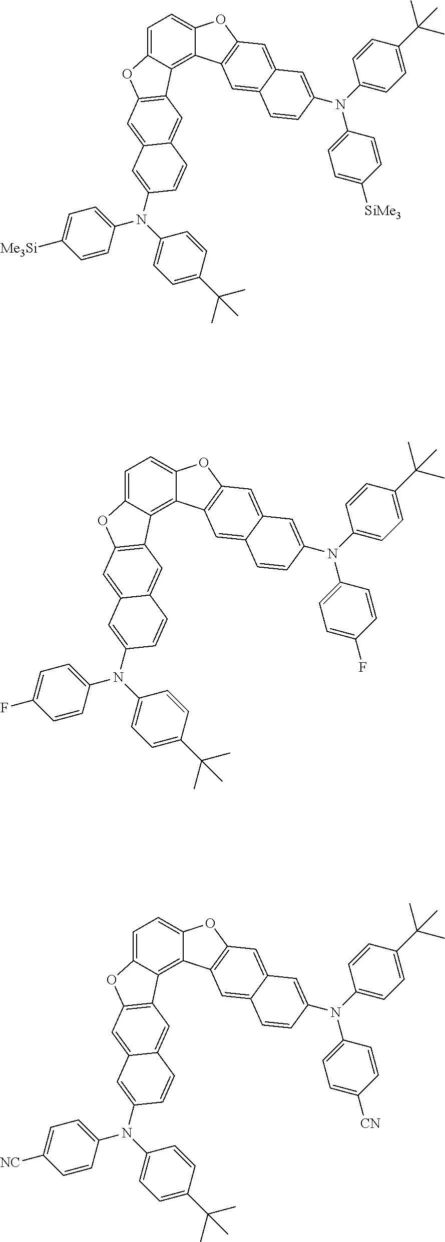 [SMILES: CC(C)(C)c1ccc(N(c2ccc([Si](C)(C)C)cc2)c2ccc3cc4c(cc3c2)oc2ccc3oc5cc6cc(N(c7ccc(C(C)(C)C)cc7)c7ccc([Si](C)(C)C)cc7)ccc6cc5c3c24)cc1 ; CC(C)(C)c1ccc(N(c2ccc(F)cc2)c2ccc3cc4c(cc3c2)oc2ccc3oc5cc6cc(N(c7ccc(F)cc7)c7ccc(C(C)(C)C)cc7)ccc6cc5c3c24)cc1 ; CC(C)(C)c1ccc(N(c2ccc(C#N)cc2)c2ccc3cc4c(cc3c2)oc2ccc3oc5cc6cc(N(c7ccc(C#N)cc7)c7ccc(C(C)(C)C)cc7)ccc6cc5c3c24)cc1]

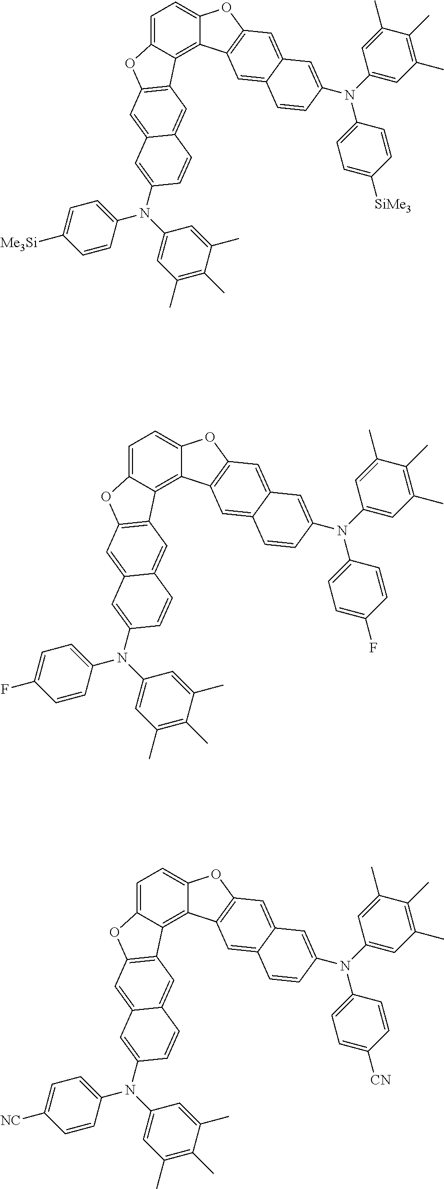 [SMILES: Cc1cc(N(c2ccc([Si](C)(C)C)cc2)c2ccc3cc4c(cc3c2)oc2ccc3oc5cc6cc(N(c7ccc([Si](C)(C)C)cc7)c7cc(C)c(C)c(C)c7)ccc6cc5c3c24)cc(C)c1C ; Cc1cc(N(c2ccc(F)cc2)c2ccc3cc4c(cc3c2)oc2ccc3oc5cc6cc(N(c7ccc(F)cc7)c7cc(C)c(C)c(C)c7)ccc6cc5c3c24)cc(C)c1C ; Cc1cc(N(c2ccc(C#N)cc2)c2ccc3cc4c(cc3c2)oc2ccc3oc5cc6cc(N(c7ccc(C#N)cc7)c7cc(C)c(C)c(C)c7)ccc6cc5c3c24)cc(C)c1C]

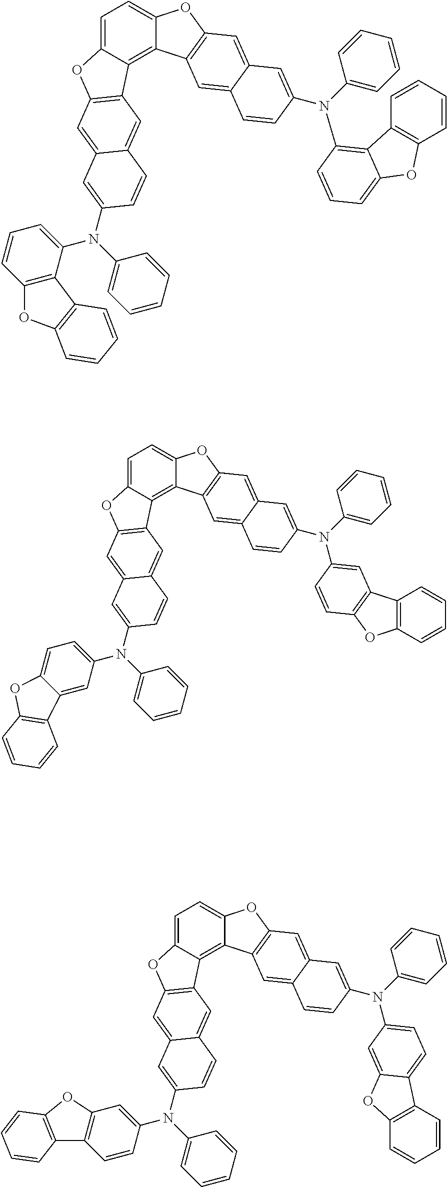 [SMILES: c1ccc(N(c2ccc3cc4c(cc3c2)oc2ccc3oc5cc6cc(N(c7ccccc7)c7cccc8oc9ccccc9c78)ccc6cc5c3c24)c2cccc3oc4ccccc4c23)cc1 ; c1ccc(N(c2ccc3cc4c(cc3c2)oc2ccc3oc5cc6cc(N(c7ccccc7)c7ccc8oc9ccccc9c8c7)ccc6cc5c3c24)c2ccc3oc4ccccc4c3c2)cc1 ; c1ccc(N(c2ccc3cc4c(cc3c2)oc2ccc3oc5cc6cc(N(c7ccccc7)c7ccc8c(c7)oc7ccccc78)ccc6cc5c3c24)c2ccc3c(c2)oc2ccccc23)cc1]

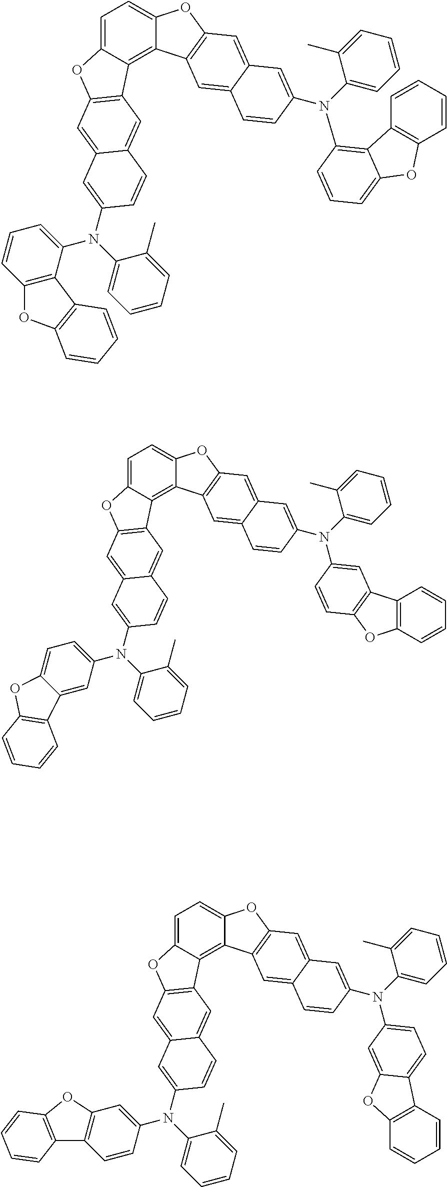 [SMILES: Cc1ccccc1N(c1ccc2cc3c(cc2c1)oc1ccc2oc4cc5cc(N(c6ccccc6C)c6cccc7oc8ccccc8c67)ccc5cc4c2c13)c1cccc2oc3ccccc3c12 ; Cc1ccccc1N(c1ccc2cc3c(cc2c1)oc1ccc2oc4cc5cc(N(c6ccc7oc8ccccc8c7c6)c6ccccc6C)ccc5cc4c2c13)c1ccc2oc3ccccc3c2c1 ; Cc1ccccc1N(c1ccc2cc3c(cc2c1)oc1ccc2oc4cc5cc(N(c6ccc7c(c6)oc6ccccc67)c6ccccc6C)ccc5cc4c2c13)c1ccc2c(c1)oc1ccccc12]

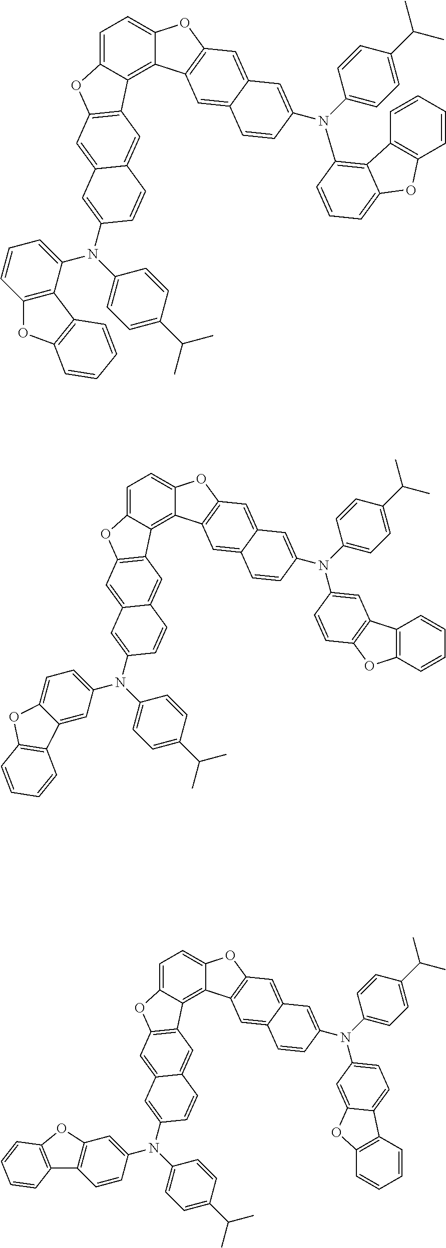 [SMILES: CC(C)c1ccc(N(c2ccc3cc4c(cc3c2)oc2ccc3oc5cc6cc(N(c7ccc(C(C)C)cc7)c7cccc8oc9ccccc9c78)ccc6cc5c3c24)c2cccc3oc4ccccc4c23)cc1 ; CC(C)c1ccc(N(c2ccc3cc4c(cc3c2)oc2ccc3oc5cc6cc(N(c7ccc(C(C)C)cc7)c7ccc8oc9ccccc9c8c7)ccc6cc5c3c24)c2ccc3oc4ccccc4c3c2)cc1 ; CC(C)c1ccc(N(c2ccc3cc4c(cc3c2)oc2ccc3oc5cc6cc(N(c7ccc(C(C)C)cc7)c7ccc8c(c7)oc7ccccc78)ccc6cc5c3c24)c2ccc3c(c2)oc2ccccc23)cc1]

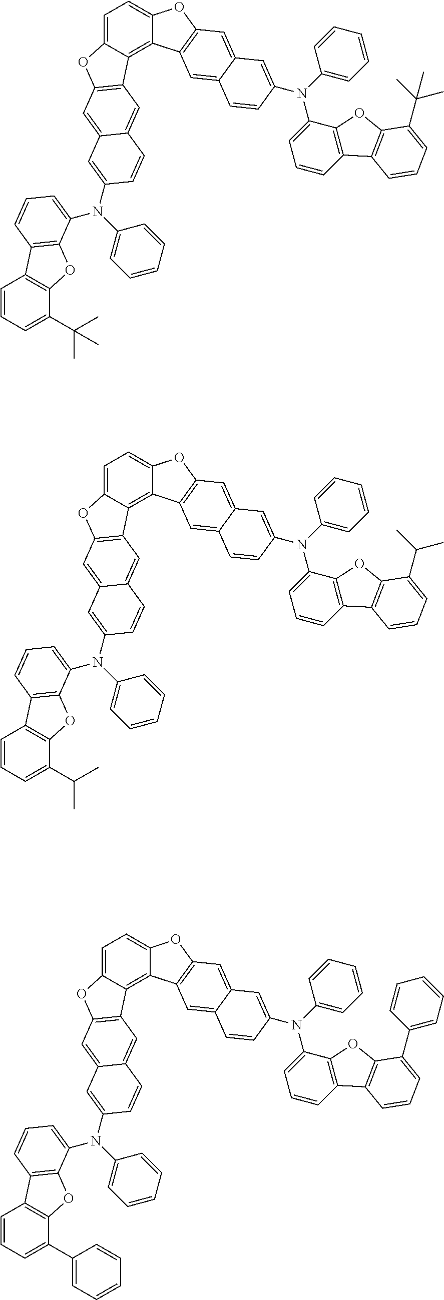 [SMILES: CC(C)(C)c1cccc2c1oc1c(N(c3ccccc3)c3ccc4cc5c(cc4c3)oc3ccc4oc6cc7cc(N(c8ccccc8)c8cccc9c8oc8c(C(C)(C)C)cccc89)ccc7cc6c4c35)cccc12 ; CC(C)c1cccc2c1oc1c(N(c3ccccc3)c3ccc4cc5c(cc4c3)oc3ccc4oc6cc7cc(N(c8ccccc8)c8cccc9c8oc8c(C(C)C)cccc89)ccc7cc6c4c35)cccc12 ; c1ccc(-c2cccc3c2oc2c(N(c4ccccc4)c4ccc5cc6c(cc5c4)oc4ccc5oc7cc8cc(N(c9ccccc9)c9cccc%10c9oc9c(-c%11ccccc%11)cccc9%10)ccc8cc7c5c46)cccc23)cc1]

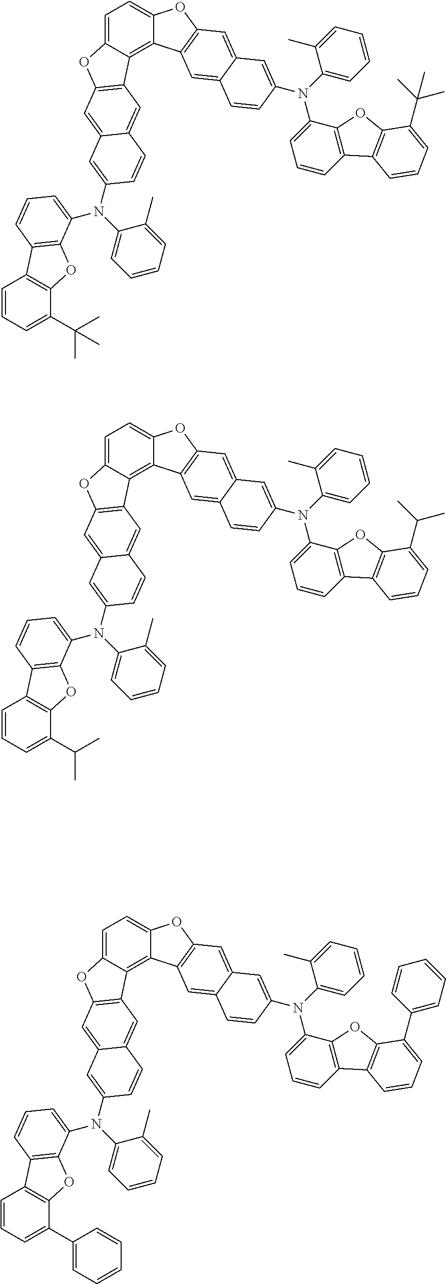 [SMILES: Cc1ccccc1N(c1ccc2cc3c(cc2c1)oc1ccc2oc4cc5cc(N(c6ccccc6C)c6cccc7c6oc6c(C(C)(C)C)cccc67)ccc5cc4c2c13)c1cccc2c1oc1c(C(C)(C)C)cccc12 ; Cc1ccccc1N(c1ccc2cc3c(cc2c1)oc1ccc2oc4cc5cc(N(c6ccccc6C)c6cccc7c6oc6c(C(C)C)cccc67)ccc5cc4c2c13)c1cccc2c1oc1c(C(C)C)cccc12 ; Cc1ccccc1N(c1ccc2cc3c(cc2c1)oc1ccc2oc4cc5cc(N(c6ccccc6C)c6cccc7c6oc6c(-c8ccccc8)cccc67)ccc5cc4c2c13)c1cccc2c1oc1c(-c3ccccc3)cccc12]

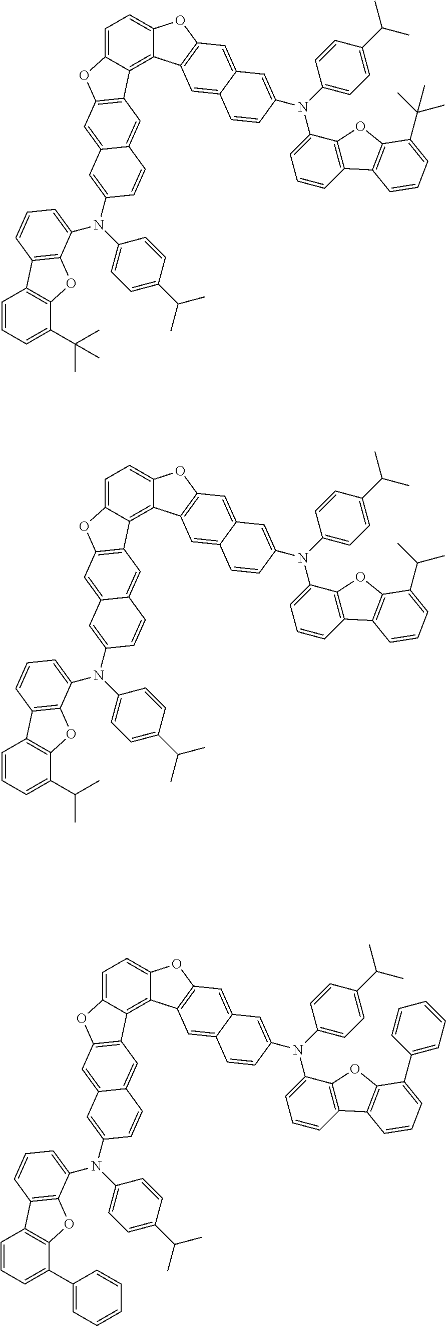 [SMILES: CC(C)c1ccc(N(c2ccc3cc4c(cc3c2)oc2ccc3oc5cc6cc(N(c7ccc(C(C)C)cc7)c7cccc8c7oc7c(C(C)(C)C)cccc78)ccc6cc5c3c24)c2cccc3c2oc2c(C(C)(C)C)cccc23)cc1 ; CC(C)c1ccc(N(c2ccc3cc4c(cc3c2)oc2ccc3oc5cc6cc(N(c7ccc(C(C)C)cc7)c7cccc8c7oc7c(C(C)C)cccc78)ccc6cc5c3c24)c2cccc3c2oc2c(C(C)C)cccc23)cc1 ; CC(C)c1ccc(N(c2ccc3cc4c(cc3c2)oc2ccc3oc5cc6cc(N(c7ccc(C(C)C)cc7)c7cccc8c7oc7c(-c9ccccc9)cccc78)ccc6cc5c3c24)c2cccc3c2oc2c(-c4ccccc4)cccc23)cc1]

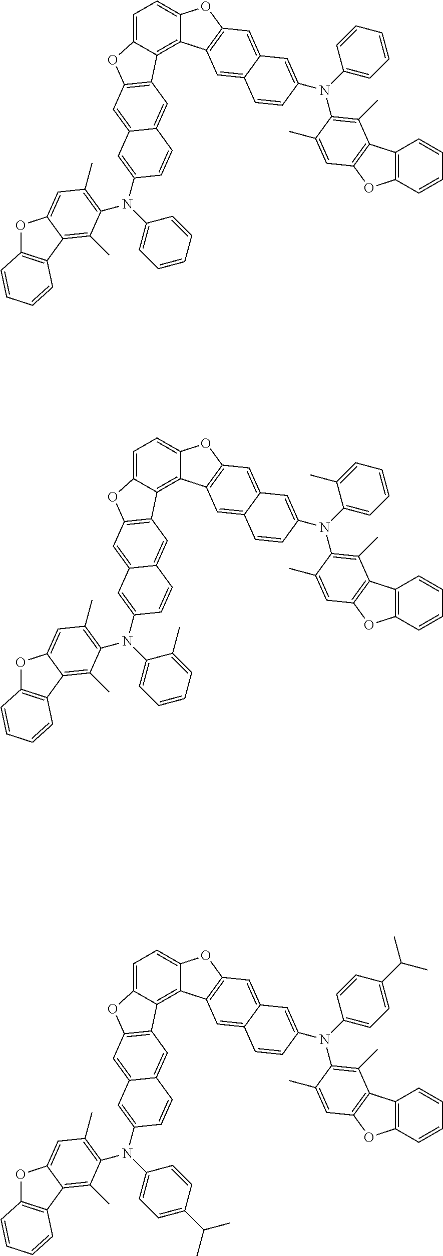 [SMILES: Cc1cc2oc3ccccc3c2c(C)c1N(c1ccccc1)c1ccc2cc3c(cc2c1)oc1ccc2oc4cc5cc(N(c6ccccc6)c6c(C)cc7oc8ccccc8c7c6C)ccc5cc4c2c13 ; Cc1ccccc1N(c1ccc2cc3c(cc2c1)oc1ccc2oc4cc5cc(N(c6ccccc6C)c6c(C)cc7oc8ccccc8c7c6C)ccc5cc4c2c13)c1c(C)cc2oc3ccccc3c2c1C ; Cc1cc2oc3ccccc3c2c(C)c1N(c1ccc(C(C)C)cc1)c1ccc2cc3c(cc2c1)oc1ccc2oc4cc5cc(N(c6ccc(C(C)C)cc6)c6c(C)cc7oc8ccccc8c7c6C)ccc5cc4c2c13]

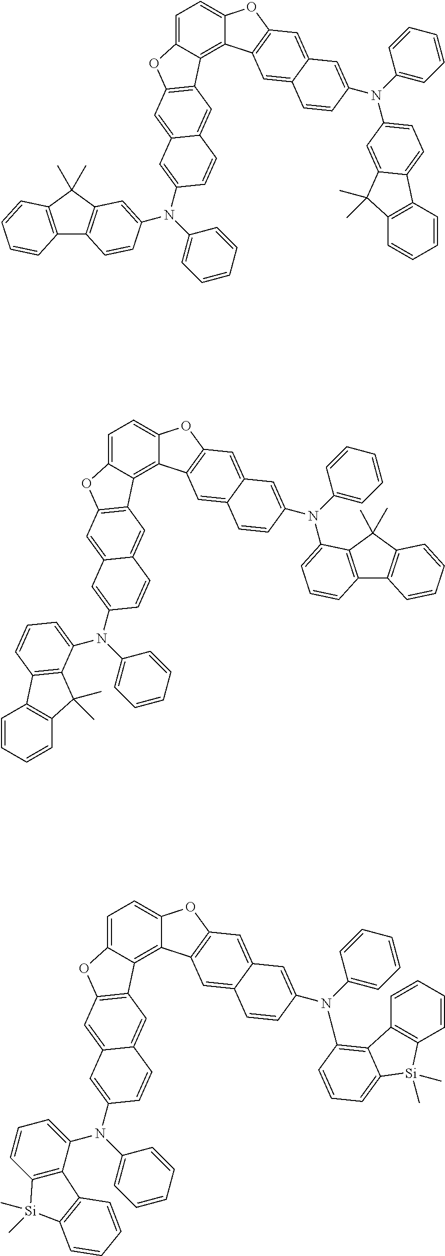 [SMILES: CC1(C)c2ccccc2-c2ccc(N(c3ccccc3)c3ccc4cc5c(cc4c3)oc3ccc4oc6cc7cc(N(c8ccccc8)c8ccc9c(c8)C(C)(C)c8ccccc8-9)ccc7cc6c4c35)cc21 ; CC1(C)c2ccccc2-c2cccc(N(c3ccccc3)c3ccc4cc5c(cc4c3)oc3ccc4oc6cc7cc(N(c8ccccc8)c8cccc9c8C(C)(C)c8ccccc8-9)ccc7cc6c4c35)c21 ; C[Si]1(C)c2ccccc2-c2c(N(c3ccccc3)c3ccc4cc5c(cc4c3)oc3ccc4oc6cc7cc(N(c8ccccc8)c8cccc9c8-c8ccccc8[Si]9(C)C)ccc7cc6c4c35)cccc21]

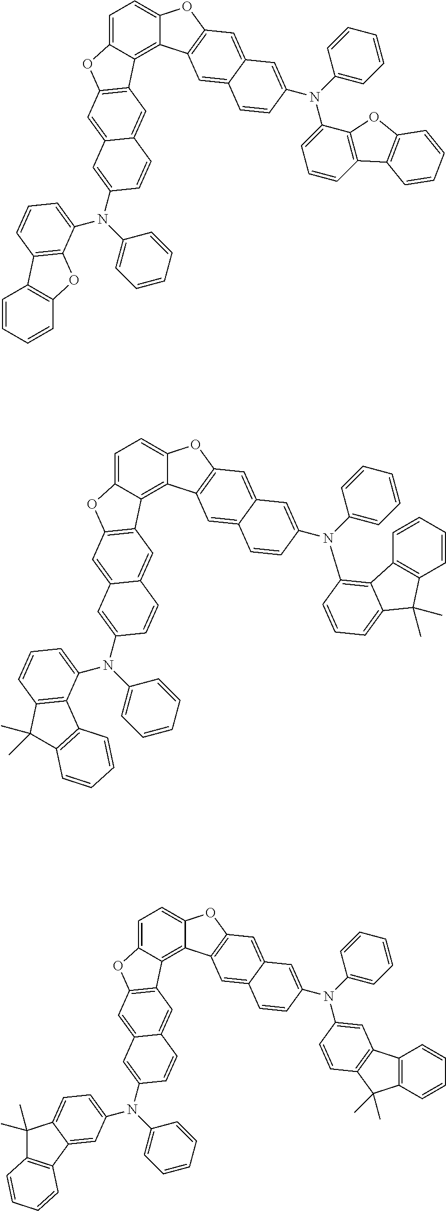 [SMILES: c1ccc(N(c2ccc3cc4c(cc3c2)oc2ccc3oc5cc6cc(N(c7ccccc7)c7cccc8c7oc7ccccc78)ccc6cc5c3c24)c2cccc3c2oc2ccccc23)cc1 ; CC1(C)c2ccccc2-c2c(N(c3ccccc3)c3ccc4cc5c(cc4c3)oc3ccc4oc6cc7cc(N(c8ccccc8)c8cccc9c8-c8ccccc8C9(C)C)ccc7cc6c4c35)cccc21 ; CC1(C)c2ccccc2-c2cc(N(c3ccccc3)c3ccc4cc5c(cc4c3)oc3ccc4oc6cc7cc(N(c8ccccc8)c8ccc9c(c8)-c8ccccc8C9(C)C)ccc7cc6c4c35)ccc21]

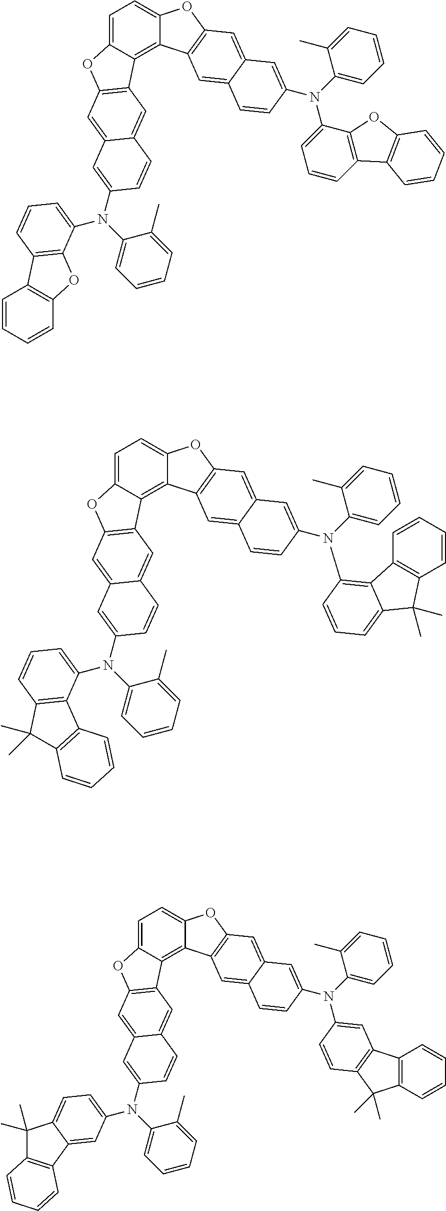 [SMILES: Cc1ccccc1N(c1ccc2cc3c(cc2c1)oc1ccc2oc4cc5cc(N(c6ccccc6C)c6cccc7c6oc6ccccc67)ccc5cc4c2c13)c1cccc2c1oc1ccccc12 ; Cc1ccccc1N(c1ccc2cc3c(cc2c1)oc1ccc2oc4cc5cc(N(c6ccccc6C)c6cccc7c6-c6ccccc6C7(C)C)ccc5cc4c2c13)c1cccc2c1-c1ccccc1C2(C)C ; Cc1ccccc1N(c1ccc2c(c1)-c1ccccc1C2(C)C)c1ccc2cc3c(cc2c1)oc1ccc2oc4cc5cc(N(c6ccc7c(c6)-c6ccccc6C7(C)C)c6ccccc6C)ccc5cc4c2c13]

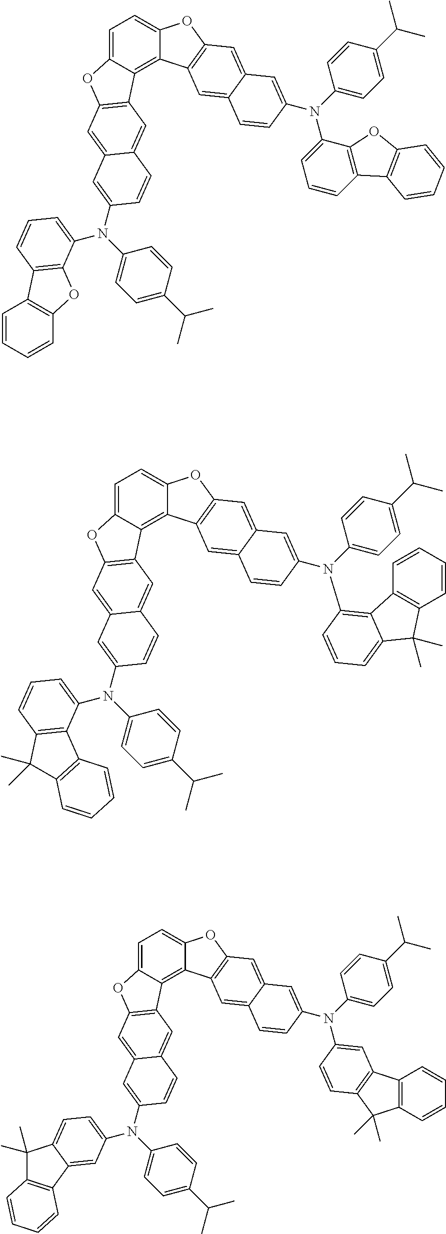 [SMILES: CC(C)c1ccc(N(c2ccc3cc4c(cc3c2)oc2ccc3oc5cc6cc(N(c7ccc(C(C)C)cc7)c7cccc8c7oc7ccccc78)ccc6cc5c3c24)c2cccc3c2oc2ccccc23)cc1 ; CC(C)c1ccc(N(c2ccc3cc4c(cc3c2)oc2ccc3oc5cc6cc(N(c7ccc(C(C)C)cc7)c7cccc8c7-c7ccccc7C8(C)C)ccc6cc5c3c24)c2cccc3c2-c2ccccc2C3(C)C)cc1 ; CC(C)c1ccc(N(c2ccc3c(c2)-c2ccccc2C3(C)C)c2ccc3cc4c(cc3c2)oc2ccc3oc5cc6cc(N(c7ccc(C(C)C)cc7)c7ccc8c(c7)-c7ccccc7C8(C)C)ccc6cc5c3c24)cc1]

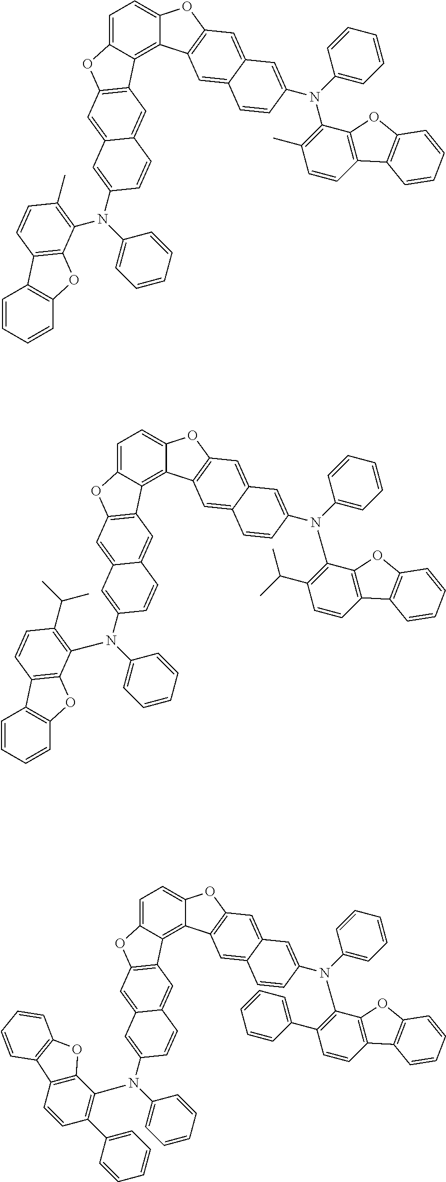 [SMILES: Cc1ccc2c(oc3ccccc32)c1N(c1ccccc1)c1ccc2cc3c(cc2c1)oc1ccc2oc4cc5cc(N(c6ccccc6)c6c(C)ccc7c6oc6ccccc67)ccc5cc4c2c13 ; CC(C)c1ccc2c(oc3ccccc32)c1N(c1ccccc1)c1ccc2cc3c(cc2c1)oc1ccc2oc4cc5cc(N(c6ccccc6)c6c(C(C)C)ccc7c6oc6ccccc67)ccc5cc4c2c13 ; c1ccc(-c2ccc3c(oc4ccccc43)c2N(c2ccccc2)c2ccc3cc4c(cc3c2)oc2ccc3oc5cc6cc(N(c7ccccc7)c7c(-c8ccccc8)ccc8c7oc7ccccc78)ccc6cc5c3c24)cc1]

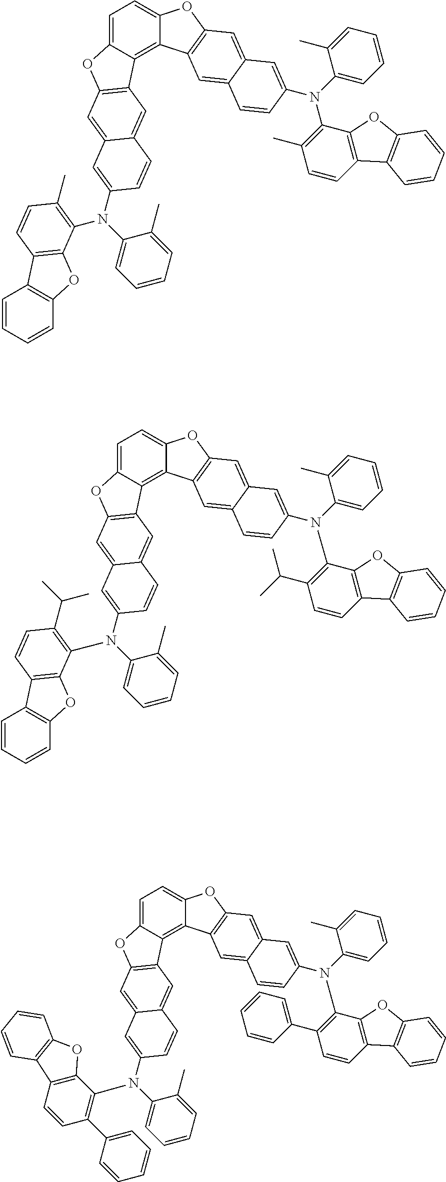 [SMILES: Cc1ccccc1N(c1ccc2cc3c(cc2c1)oc1ccc2oc4cc5cc(N(c6ccccc6C)c6c(C)ccc7c6oc6ccccc67)ccc5cc4c2c13)c1c(C)ccc2c1oc1ccccc12 ; Cc1ccccc1N(c1ccc2cc3c(cc2c1)oc1ccc2oc4cc5cc(N(c6ccccc6C)c6c(C(C)C)ccc7c6oc6ccccc67)ccc5cc4c2c13)c1c(C(C)C)ccc2c1oc1ccccc12 ; Cc1ccccc1N(c1ccc2cc3c(cc2c1)oc1ccc2oc4cc5cc(N(c6ccccc6C)c6c(-c7ccccc7)ccc7c6oc6ccccc67)ccc5cc4c2c13)c1c(-c2ccccc2)ccc2c1oc1ccccc12]

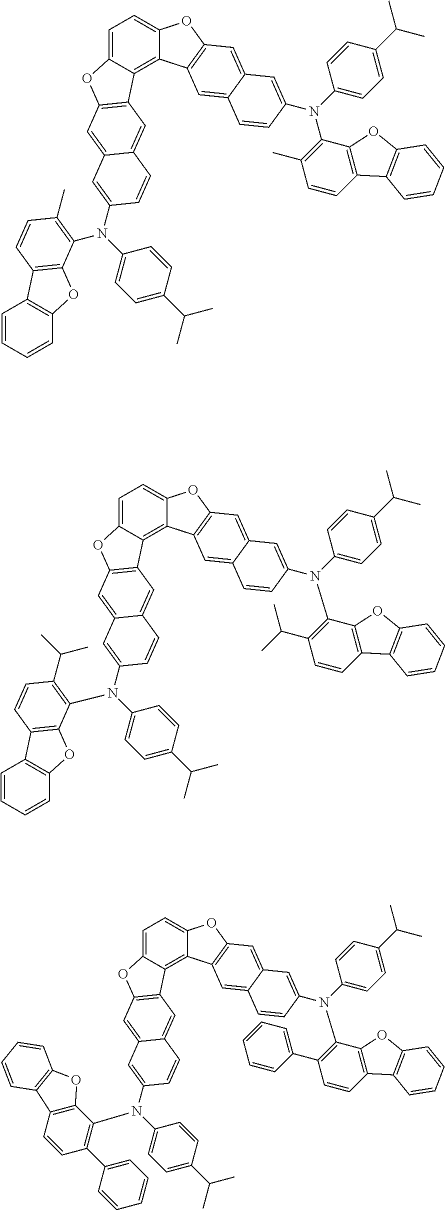 [SMILES: Cc1ccc2c(oc3ccccc32)c1N(c1ccc(C(C)C)cc1)c1ccc2cc3c(cc2c1)oc1ccc2oc4cc5cc(N(c6ccc(C(C)C)cc6)c6c(C)ccc7c6oc6ccccc67)ccc5cc4c2c13 ; CC(C)c1ccc(N(c2ccc3cc4c(cc3c2)oc2ccc3oc5cc6cc(N(c7ccc(C(C)C)cc7)c7c(C(C)C)ccc8c7oc7ccccc78)ccc6cc5c3c24)c2c(C(C)C)ccc3c2oc2ccccc23)cc1 ; CC(C)c1ccc(N(c2ccc3cc4c(cc3c2)oc2ccc3oc5cc6cc(N(c7ccc(C(C)C)cc7)c7c(-c8ccccc8)ccc8c7oc7ccccc78)ccc6cc5c3c24)c2c(-c3ccccc3)ccc3c2oc2ccccc23)cc1]

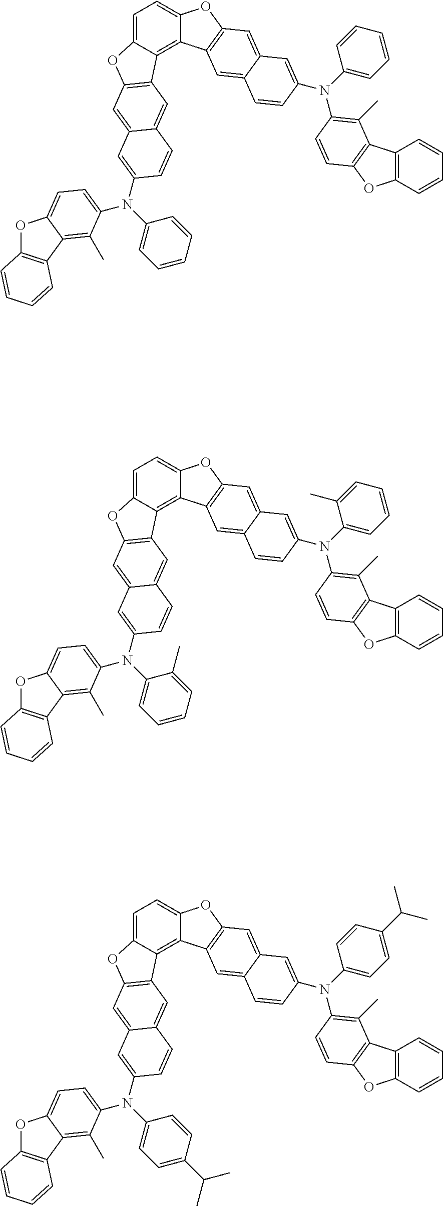 [SMILES: Cc1c(N(c2ccccc2)c2ccc3cc4c(cc3c2)oc2ccc3oc5cc6cc(N(c7ccccc7)c7ccc8oc9ccccc9c8c7C)ccc6cc5c3c24)ccc2oc3ccccc3c12 ; Cc1ccccc1N(c1ccc2cc3c(cc2c1)oc1ccc2oc4cc5cc(N(c6ccccc6C)c6ccc7oc8ccccc8c7c6C)ccc5cc4c2c13)c1ccc2oc3ccccc3c2c1C ; Cc1c(N(c2ccc(C(C)C)cc2)c2ccc3cc4c(cc3c2)oc2ccc3oc5cc6cc(N(c7ccc(C(C)C)cc7)c7ccc8oc9ccccc9c8c7C)ccc6cc5c3c24)ccc2oc3ccccc3c12]

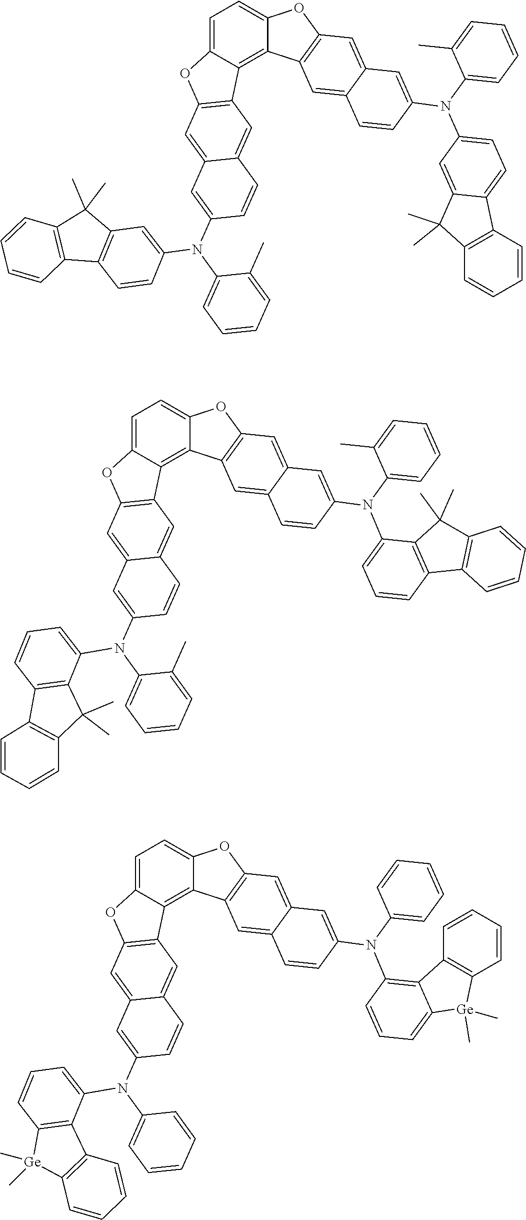

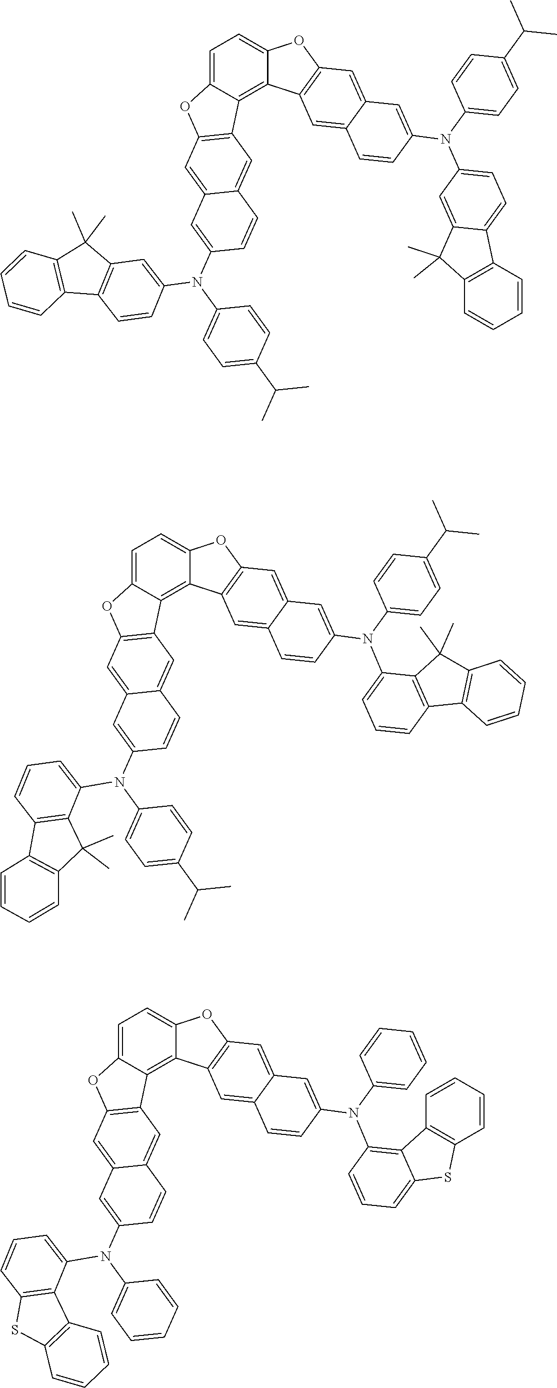 [SMILES: CC(C)c1ccc(N(c2ccc3c(c2)C(C)(C)c2ccccc2-3)c2ccc3cc4c(cc3c2)oc2ccc3oc5cc6cc(N(c7ccc(C(C)C)cc7)c7ccc8c(c7)C(C)(C)c7ccccc7-8)ccc6cc5c3c24)cc1 ; CC(C)c1ccc(N(c2ccc3cc4c(cc3c2)oc2ccc3oc5cc6cc(N(c7ccc(C(C)C)cc7)c7cccc8c7C(C)(C)c7ccccc7-8)ccc6cc5c3c24)c2cccc3c2C(C)(C)c2ccccc2-3)cc1 ; c1ccc(N(c2ccc3cc4c(cc3c2)oc2ccc3oc5cc6cc(N(c7ccccc7)c7cccc8sc9ccccc9c78)ccc6cc5c3c24)c2cccc3sc4ccccc4c23)cc1]

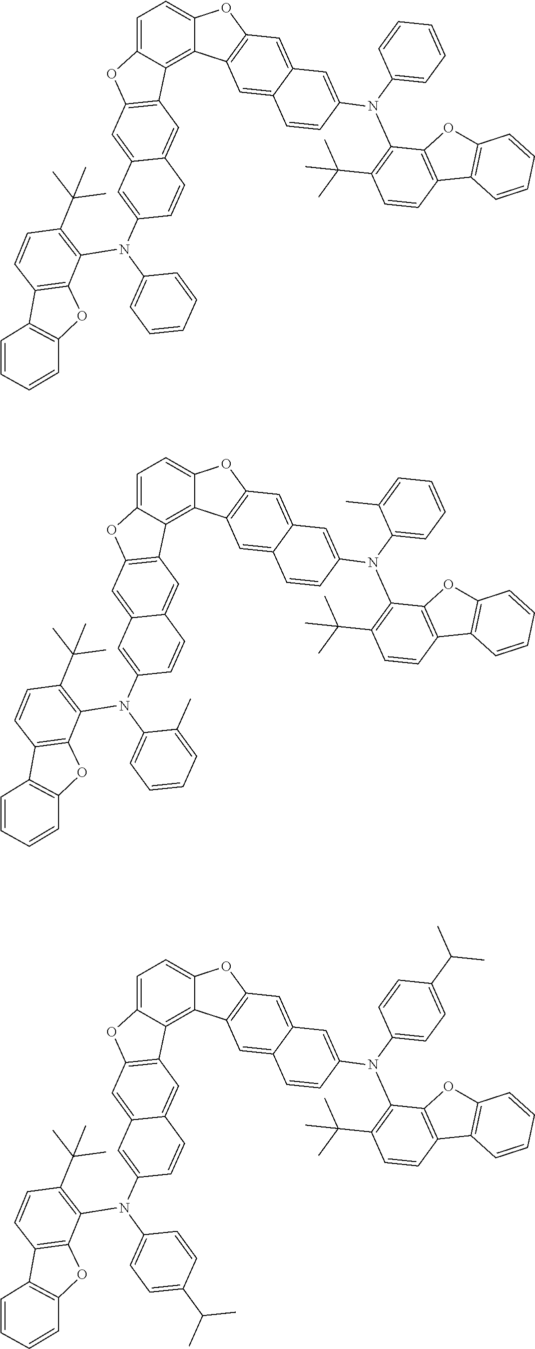 [SMILES: CC(C)(C)c1ccc2c(oc3ccccc32)c1N(c1ccccc1)c1ccc2cc3c(cc2c1)oc1ccc2oc4cc5cc(N(c6ccccc6)c6c(C(C)(C)C)ccc7c6oc6ccccc67)ccc5cc4c2c13 ; Cc1ccccc1N(c1ccc2cc3c(cc2c1)oc1ccc2oc4cc5cc(N(c6ccccc6C)c6c(C(C)(C)C)ccc7c6oc6ccccc67)ccc5cc4c2c13)c1c(C(C)(C)C)ccc2c1oc1ccccc12 ; CC(C)c1ccc(N(c2ccc3cc4c(cc3c2)oc2ccc3oc5cc6cc(N(c7ccc(C(C)C)cc7)c7c(C(C)(C)C)ccc8c7oc7ccccc78)ccc6cc5c3c24)c2c(C(C)(C)C)ccc3c2oc2ccccc23)cc1]

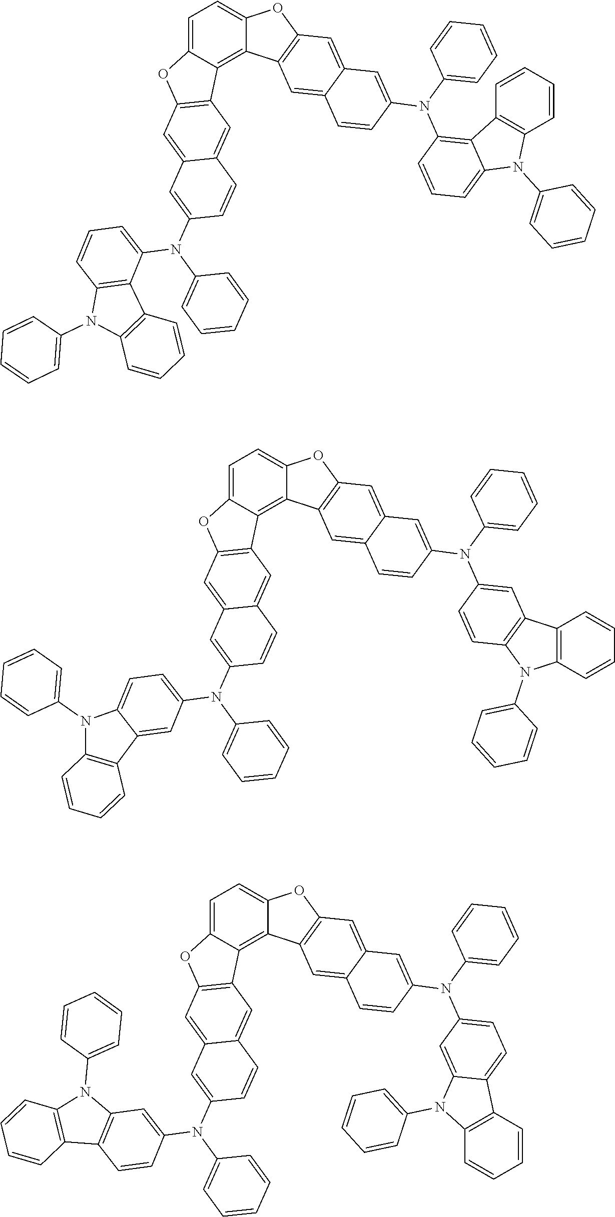 [SMILES: c1ccc(N(c2ccc3cc4c(cc3c2)oc2ccc3oc5cc6cc(N(c7ccccc7)c7cccc8c7c7ccccc7n8-c7ccccc7)ccc6cc5c3c24)c2cccc3c2c2ccccc2n3-c2ccccc2)cc1 ; c1ccc(N(c2ccc3cc4c(cc3c2)oc2ccc3oc5cc6cc(N(c7ccccc7)c7ccc8c(c7)c7ccccc7n8-c7ccccc7)ccc6cc5c3c24)c2ccc3c(c2)c2ccccc2n3-c2ccccc2)cc1 ; c1ccc(N(c2ccc3cc4c(cc3c2)oc2ccc3oc5cc6cc(N(c7ccccc7)c7ccc8c9ccccc9n(-c9ccccc9)c8c7)ccc6cc5c3c24)c2ccc3c4ccccc4n(-c4ccccc4)c3c2)cc1]

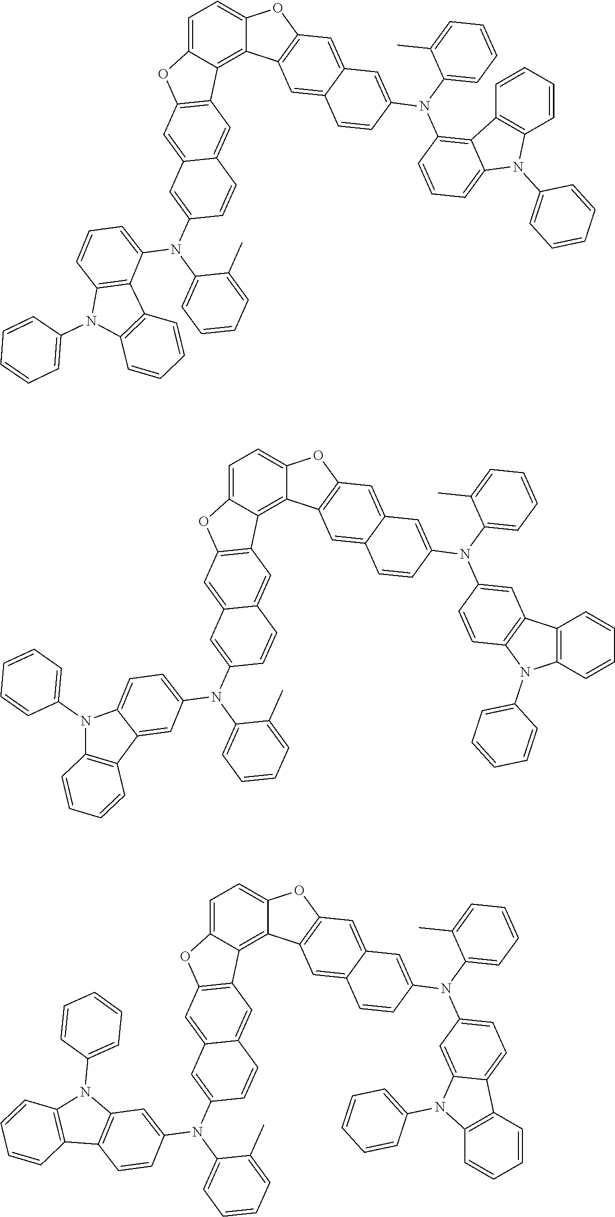 [SMILES: Cc1ccccc1N(c1ccc2cc3c(cc2c1)oc1ccc2oc4cc5cc(N(c6ccccc6C)c6cccc7c6c6ccccc6n7-c6ccccc6)ccc5cc4c2c13)c1cccc2c1c1ccccc1n2-c1ccccc1 ; Cc1ccccc1N(c1ccc2cc3c(cc2c1)oc1ccc2oc4cc5cc(N(c6ccc7c(c6)c6ccccc6n7-c6ccccc6)c6ccccc6C)ccc5cc4c2c13)c1ccc2c(c1)c1ccccc1n2-c1ccccc1 ; Cc1ccccc1N(c1ccc2cc3c(cc2c1)oc1ccc2oc4cc5cc(N(c6ccc7c8ccccc8n(-c8ccccc8)c7c6)c6ccccc6C)ccc5cc4c2c13)c1ccc2c3ccccc3n(-c3ccccc3)c2c1]

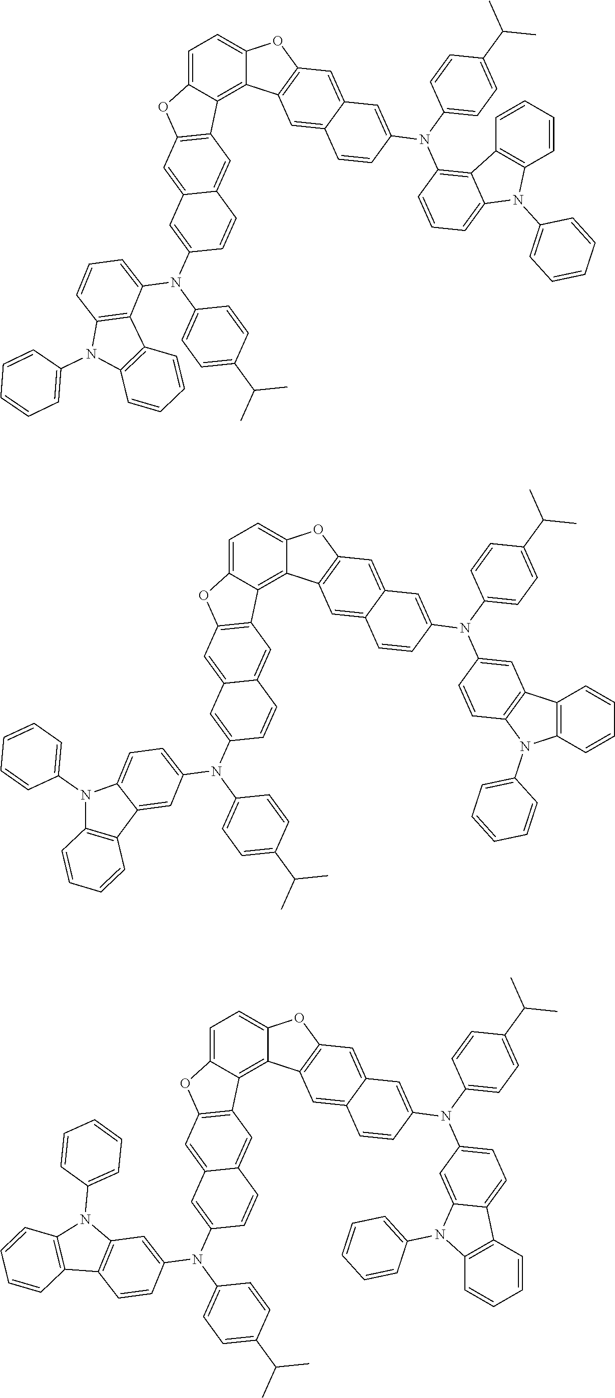 [SMILES: CC(C)c1ccc(N(c2ccc3cc4c(cc3c2)oc2ccc3oc5cc6cc(N(c7ccc(C(C)C)cc7)c7cccc8c7c7ccccc7n8-c7ccccc7)ccc6cc5c3c24)c2cccc3c2c2ccccc2n3-c2ccccc2)cc1 ; CC(C)c1ccc(N(c2ccc3cc4c(cc3c2)oc2ccc3oc5cc6cc(N(c7ccc(C(C)C)cc7)c7ccc8c(c7)c7ccccc7n8-c7ccccc7)ccc6cc5c3c24)c2ccc3c(c2)c2ccccc2n3-c2ccccc2)cc1 ; CC(C)c1ccc(N(c2ccc3cc4c(cc3c2)oc2ccc3oc5cc6cc(N(c7ccc(C(C)C)cc7)c7ccc8c9ccccc9n(-c9ccccc9)c8c7)ccc6cc5c3c24)c2ccc3c4ccccc4n(-c4ccccc4)c3c2)cc1]

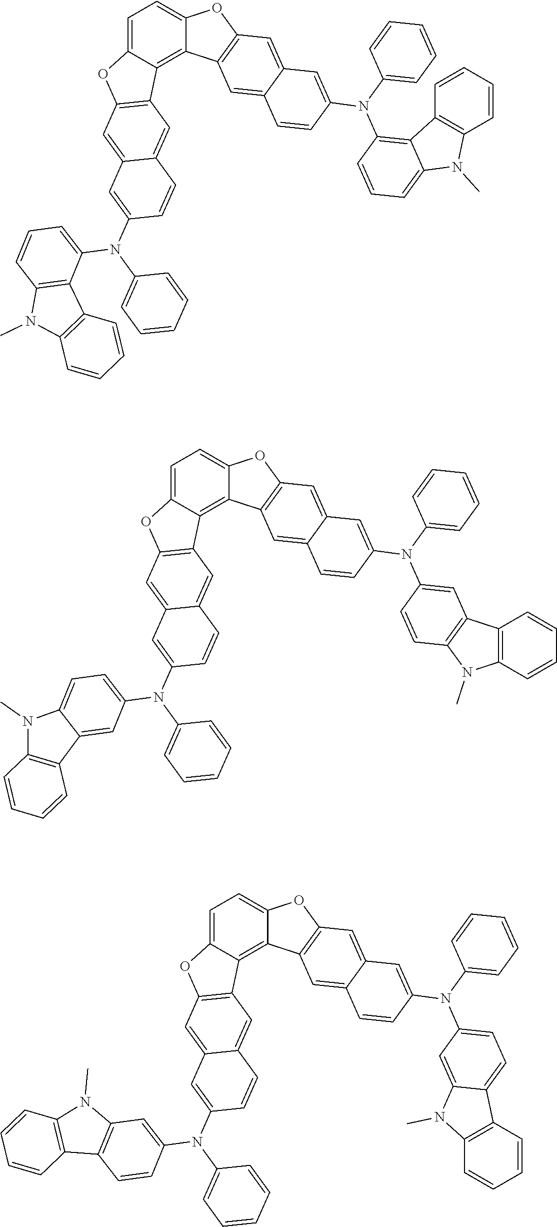 [SMILES: Cn1c2ccccc2c2c(N(c3ccccc3)c3ccc4cc5c(cc4c3)oc3ccc4oc6cc7cc(N(c8ccccc8)c8cccc9c8c8ccccc8n9C)ccc7cc6c4c35)cccc21 ; Cn1c2ccccc2c2cc(N(c3ccccc3)c3ccc4cc5c(cc4c3)oc3ccc4oc6cc7cc(N(c8ccccc8)c8ccc9c(c8)c8ccccc8n9C)ccc7cc6c4c35)ccc21 ; Cn1c2ccccc2c2ccc(N(c3ccccc3)c3ccc4cc5c(cc4c3)oc3ccc4oc6cc7cc(N(c8ccccc8)c8ccc9c%10ccccc%10n(C)c9c8)ccc7cc6c4c35)cc21]

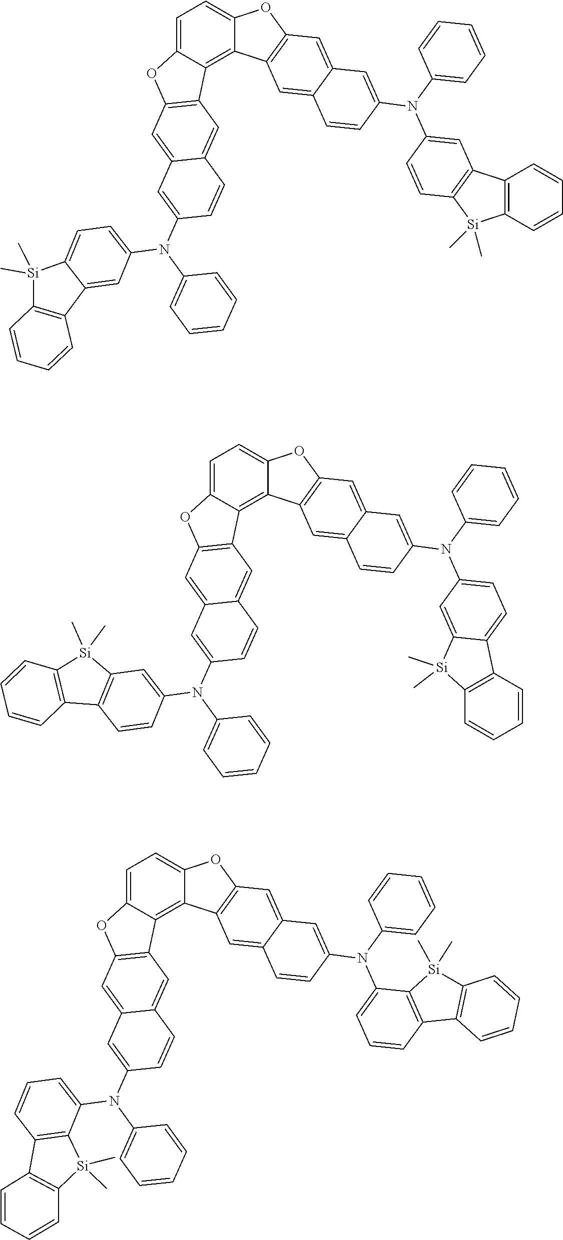 [SMILES: C[Si]1(C)c2ccccc2-c2cc(N(c3ccccc3)c3ccc4cc5c(cc4c3)oc3ccc4oc6cc7cc(N(c8ccccc8)c8ccc9c(c8)-c8ccccc8[Si]9(C)C)ccc7cc6c4c35)ccc21 ; C[Si]1(C)c2ccccc2-c2ccc(N(c3ccccc3)c3ccc4cc5c(cc4c3)oc3ccc4oc6cc7cc(N(c8ccccc8)c8ccc9c(c8)[Si](C)(C)c8ccccc8-9)ccc7cc6c4c35)cc21 ; C[Si]1(C)c2ccccc2-c2cccc(N(c3ccccc3)c3ccc4cc5c(cc4c3)oc3ccc4oc6cc7cc(N(c8ccccc8)c8cccc9c8[Si](C)(C)c8ccccc8-9)ccc7cc6c4c35)c21]

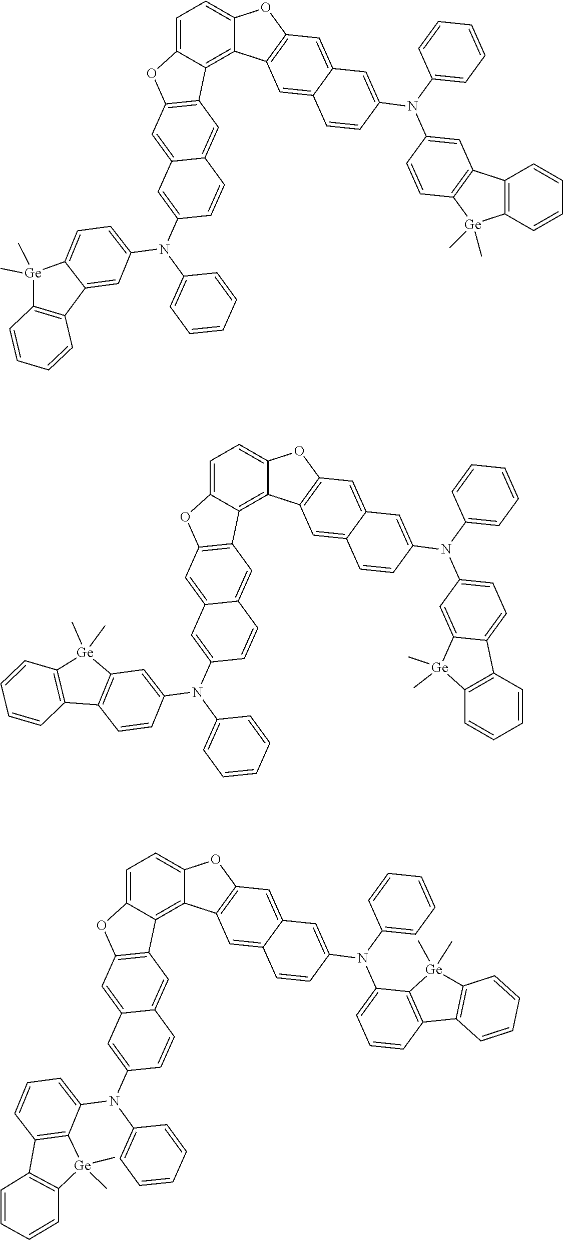

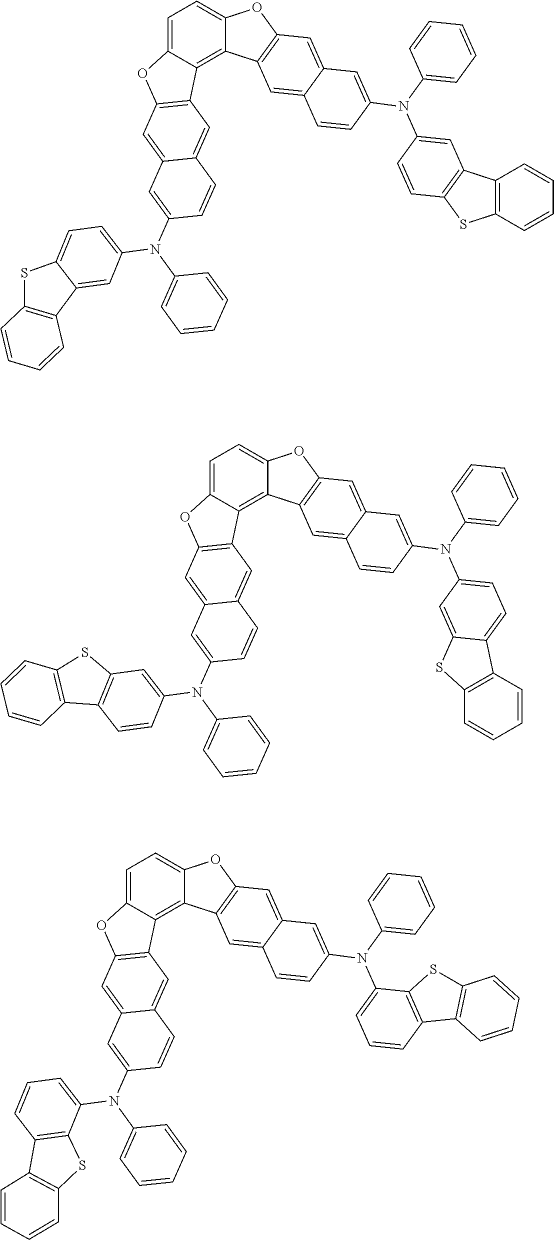 [SMILES: c1ccc(N(c2ccc3cc4c(cc3c2)oc2ccc3oc5cc6cc(N(c7ccccc7)c7ccc8sc9ccccc9c8c7)ccc6cc5c3c24)c2ccc3sc4ccccc4c3c2)cc1 ; c1ccc(N(c2ccc3cc4c(cc3c2)oc2ccc3oc5cc6cc(N(c7ccccc7)c7ccc8c(c7)sc7ccccc78)ccc6cc5c3c24)c2ccc3c(c2)sc2ccccc23)cc1 ; c1ccc(N(c2ccc3cc4c(cc3c2)oc2ccc3oc5cc6cc(N(c7ccccc7)c7cccc8c7sc7ccccc78)ccc6cc5c3c24)c2cccc3c2sc2ccccc23)cc1]

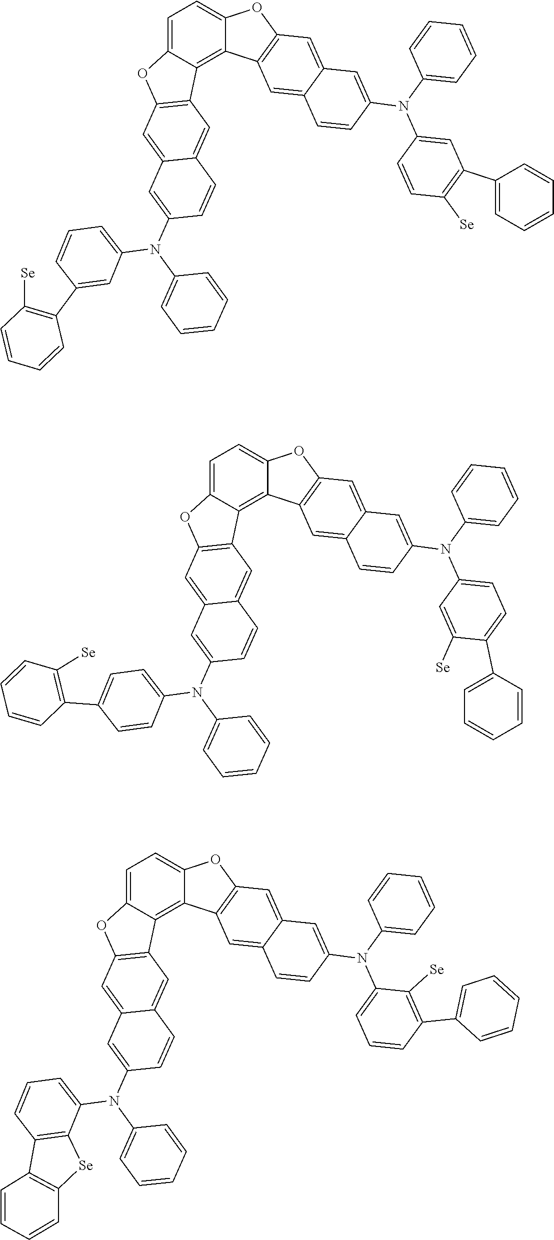

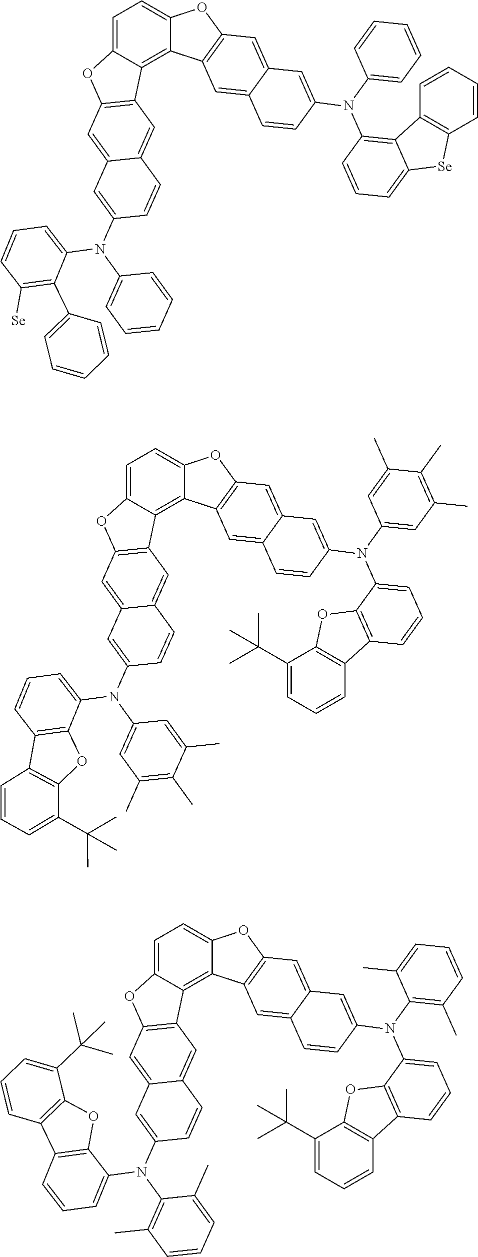

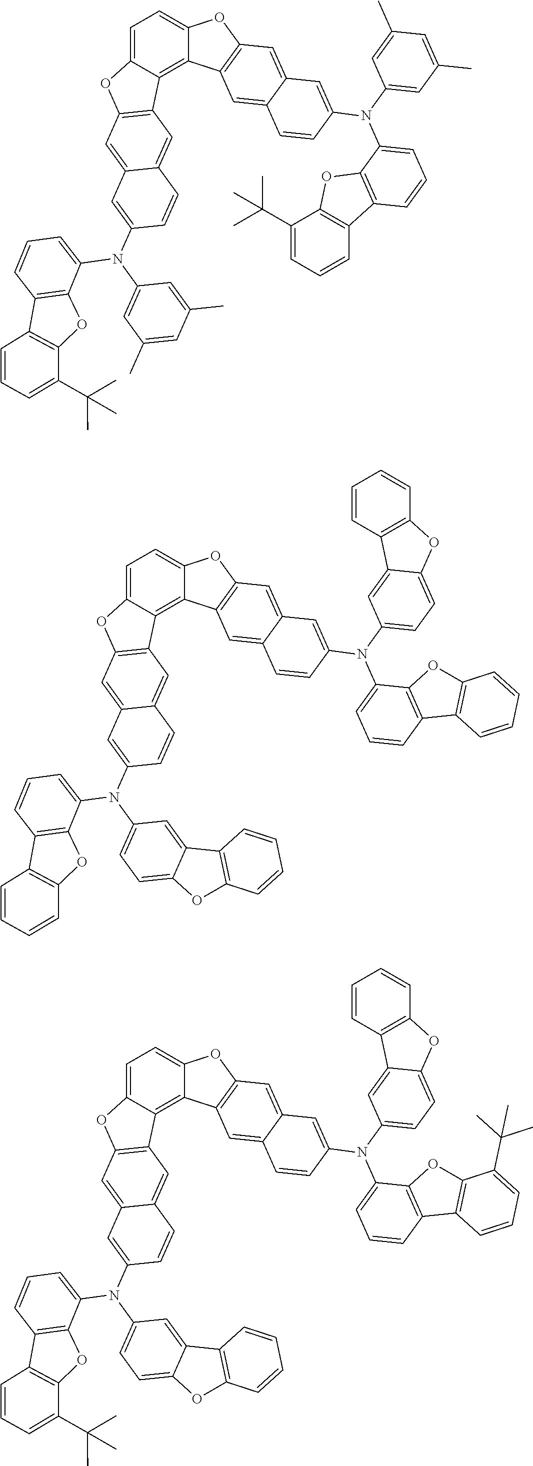 [SMILES: Cc1cc(C)cc(N(c2ccc3cc4c(cc3c2)oc2ccc3oc5cc6cc(N(c7cc(C)cc(C)c7)c7cccc8c7oc7c(C(C)(C)C)cccc78)ccc6cc5c3c24)c2cccc3c2oc2c(C(C)(C)C)cccc23)c1 ; c1ccc2c(c1)oc1ccc(N(c3ccc4cc5c(cc4c3)oc3ccc4oc6cc7cc(N(c8ccc9oc%10ccccc%10c9c8)c8cccc9c8oc8ccccc89)ccc7cc6c4c35)c3cccc4c3oc3ccccc34)cc12 ; CC(C)(C)c1cccc2c1oc1c(N(c3ccc4cc5c(cc4c3)oc3ccc4oc6cc7cc(N(c8ccc9oc%10ccccc%10c9c8)c8cccc9c8oc8c(C(C)(C)C)cccc89)ccc7cc6c4c35)c3ccc4oc5ccccc5c4c3)cccc12]

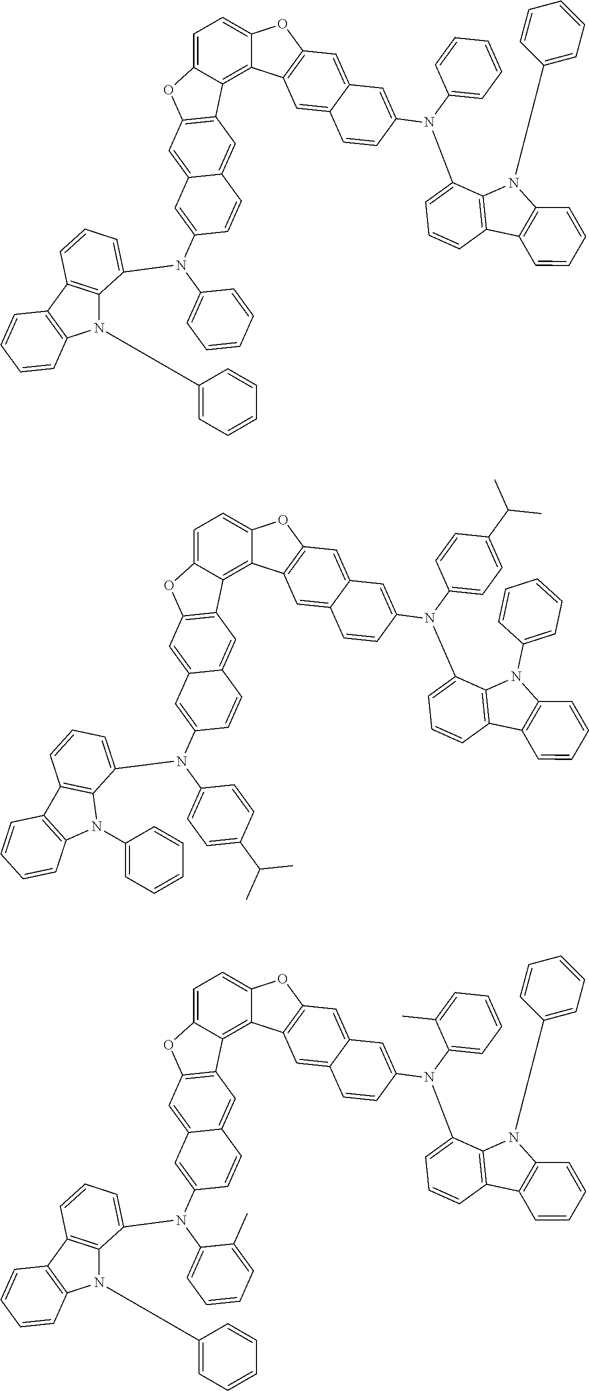 [SMILES: c1ccc(N(c2ccc3cc4c(cc3c2)oc2ccc3oc5cc6cc(N(c7ccccc7)c7cccc8c9ccccc9n(-c9ccccc9)c78)ccc6cc5c3c24)c2cccc3c4ccccc4n(-c4ccccc4)c23)cc1 ; CC(C)c1ccc(N(c2ccc3cc4c(cc3c2)oc2ccc3oc5cc6cc(N(c7ccc(C(C)C)cc7)c7cccc8c9ccccc9n(-c9ccccc9)c78)ccc6cc5c3c24)c2cccc3c4ccccc4n(-c4ccccc4)c23)cc1 ; Cc1ccccc1N(c1ccc2cc3c(cc2c1)oc1ccc2oc4cc5cc(N(c6ccccc6C)c6cccc7c8ccccc8n(-c8ccccc8)c67)ccc5cc4c2c13)c1cccc2c3ccccc3n(-c3ccccc3)c12]

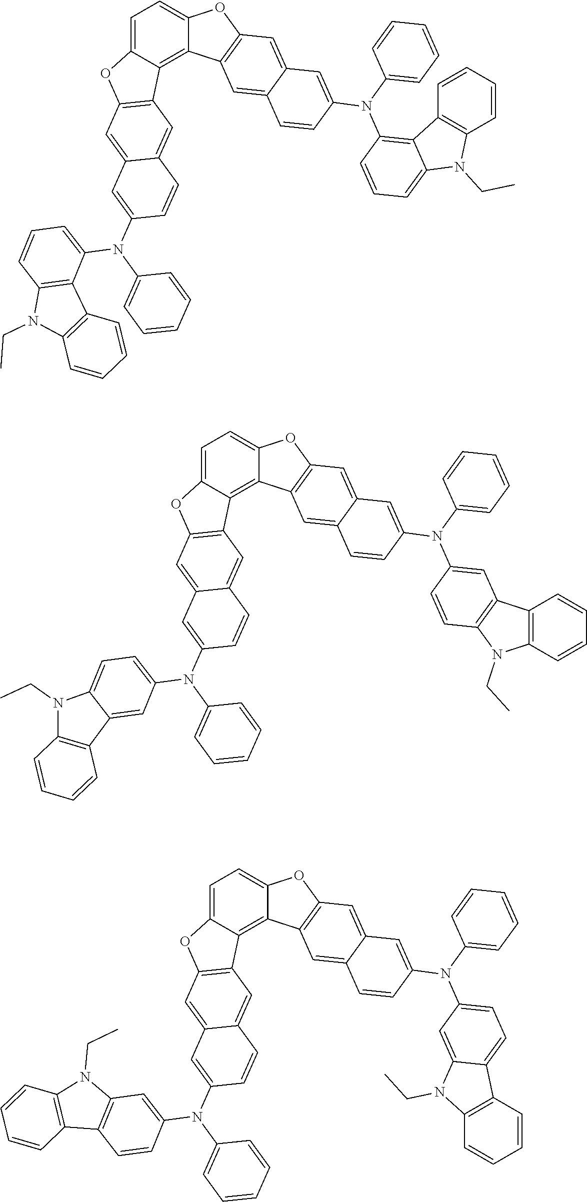

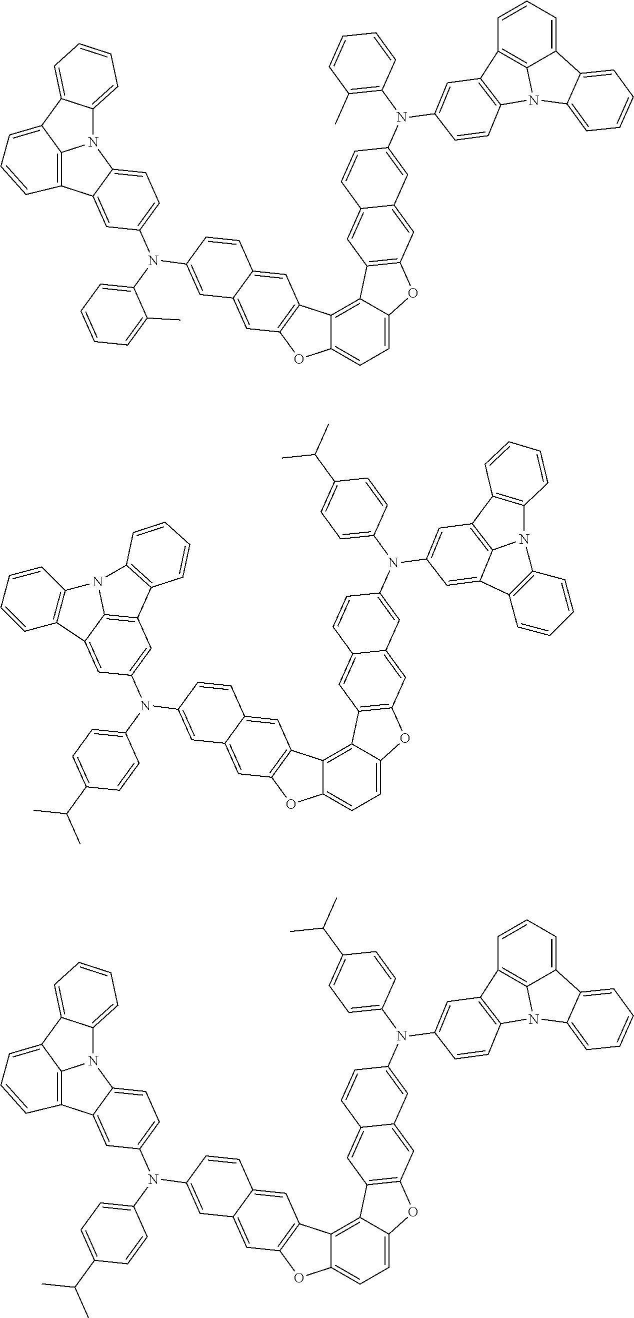 [SMILES: Cc1ccccc1N(c1ccc2cc3c(cc2c1)oc1ccc2oc4cc5cc(N(c6ccc7c(c6)c6cccc8c9ccccc9n7c86)c6ccccc6C)ccc5cc4c2c13)c1ccc2c(c1)c1cccc3c4ccccc4n2c31 ; CC(C)c1ccc(N(c2ccc3cc4c(cc3c2)oc2ccc3oc5cc6cc(N(c7ccc(C(C)C)cc7)c7cc8c9ccccc9n9c%10ccccc%10c(c7)c89)ccc6cc5c3c24)c2cc3c4ccccc4n4c5ccccc5c(c2)c34)cc1 ; CC(C)c1ccc(N(c2ccc3cc4c(cc3c2)oc2ccc3oc5cc6cc(N(c7ccc(C(C)C)cc7)c7ccc8c(c7)c7cccc9c%10ccccc%10n8c97)ccc6cc5c3c24)c2ccc3c(c2)c2cccc4c5ccccc5n3c42)cc1]

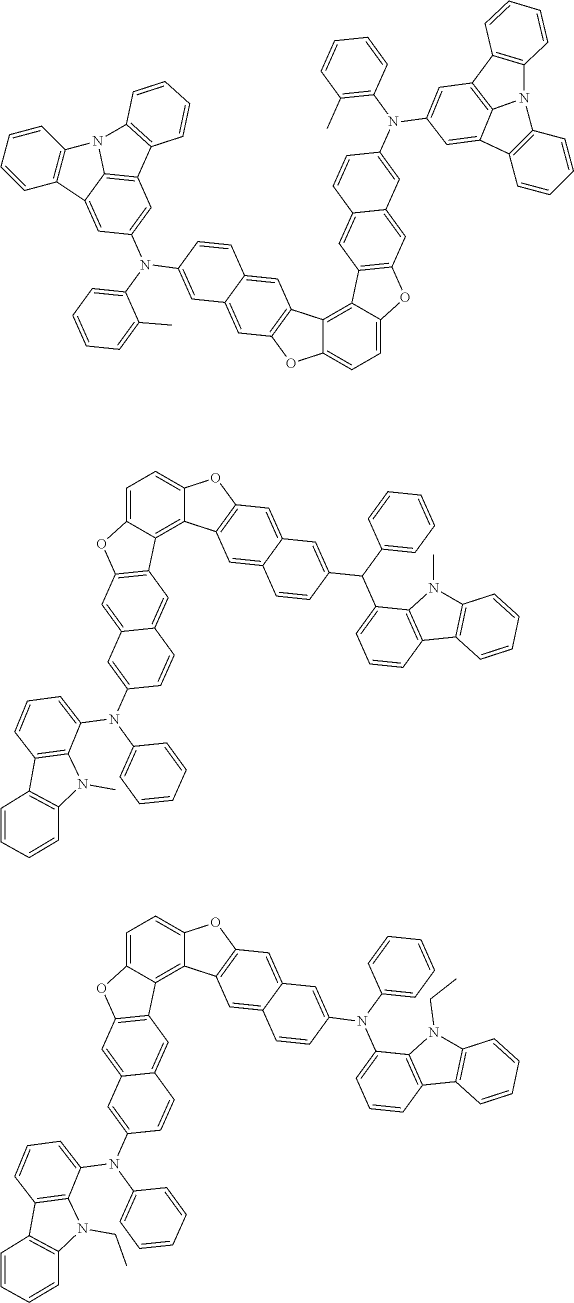 [SMILES: Cc1ccccc1N(c1ccc2cc3c(cc2c1)oc1ccc2oc4cc5cc(N(c6cc7c8ccccc8n8c9ccccc9c(c6)c78)c6ccccc6C)ccc5cc4c2c13)c1cc2c3ccccc3n3c4ccccc4c(c1)c23 ; Cn1c2ccccc2c2cccc(C(c3ccccc3)c3ccc4cc5c(cc4c3)oc3ccc4oc6cc7cc(N(c8ccccc8)c8cccc9c%10ccccc%10n(C)c89)ccc7cc6c4c35)c21 ; CCn1c2ccccc2c2cccc(N(c3ccccc3)c3ccc4cc5c(cc4c3)oc3ccc4oc6cc7cc(N(c8ccccc8)c8cccc9c%10ccccc%10n(CC)c89)ccc7cc6c4c35)c21]

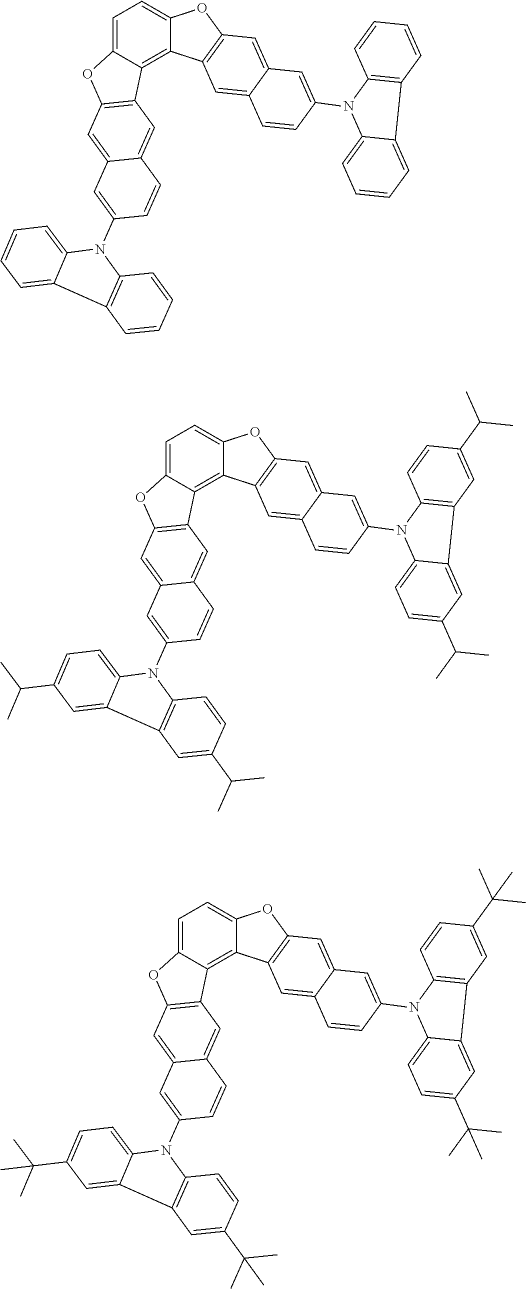

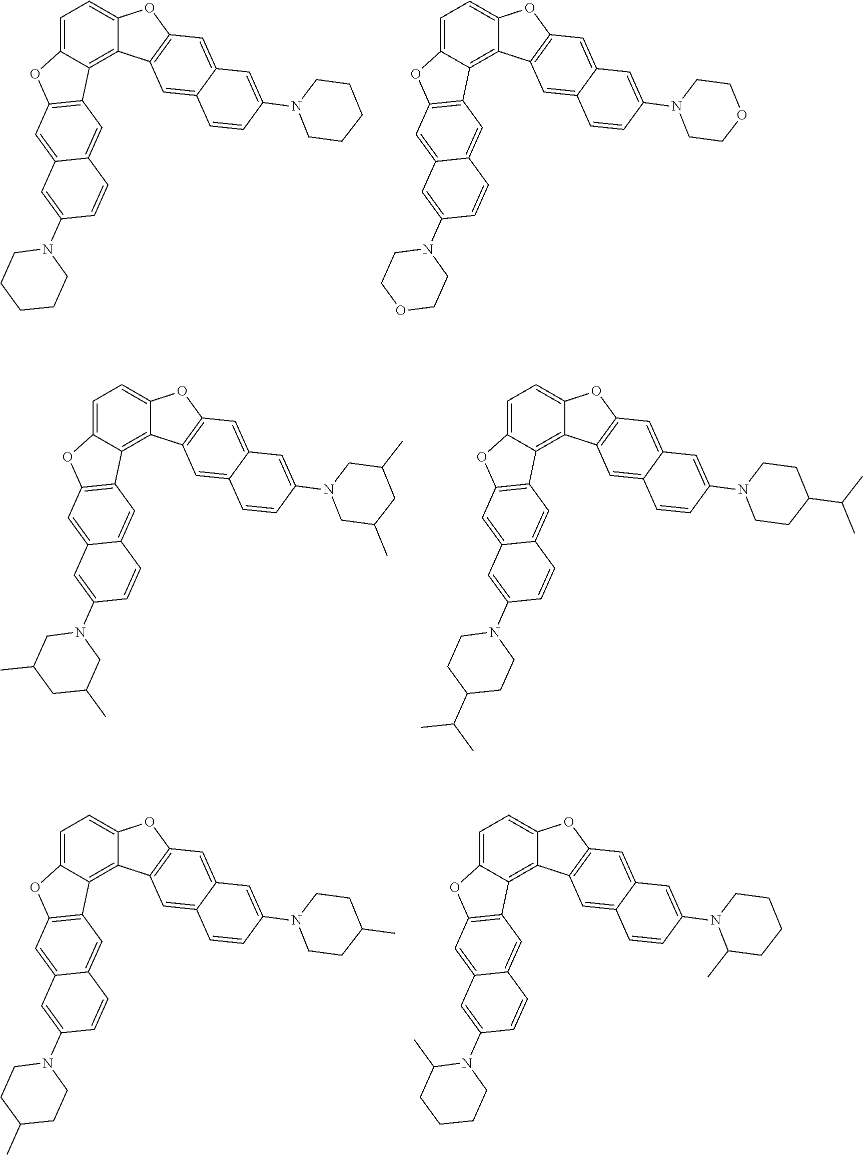

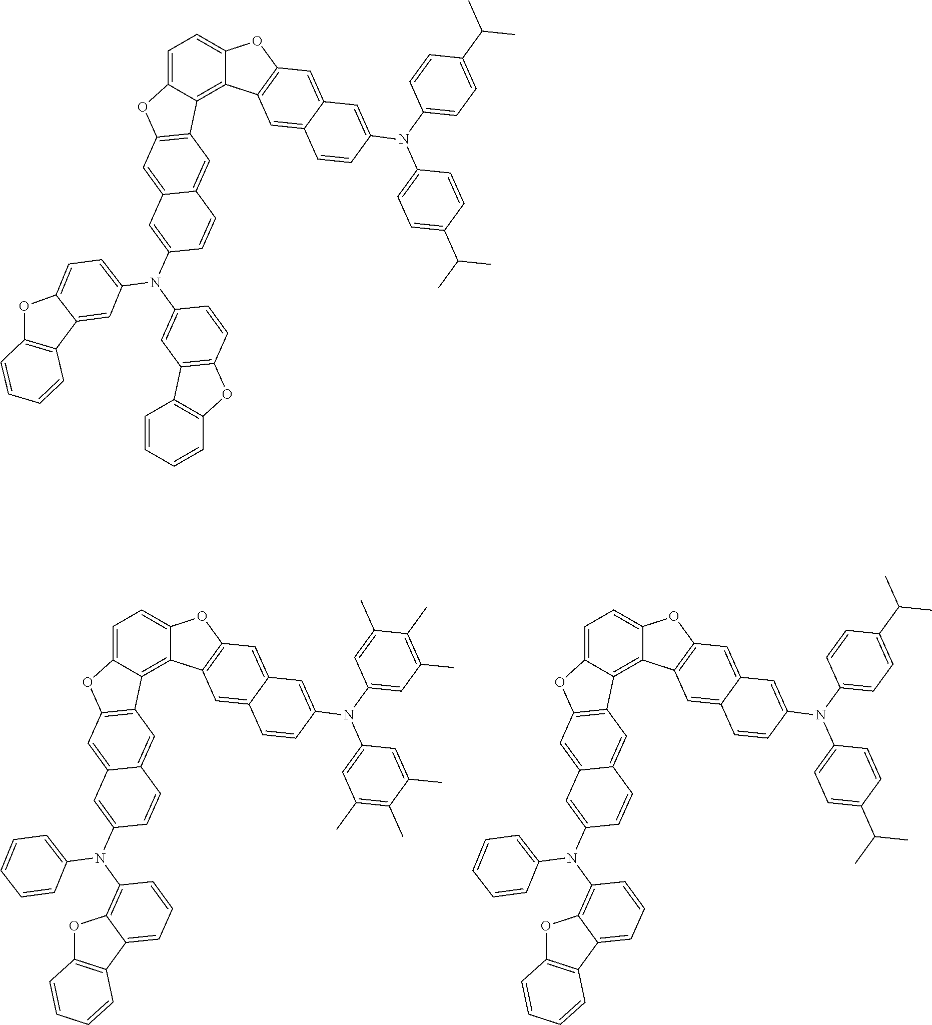 [SMILES: CC(C)c1ccc(N(c2ccc(C(C)C)cc2)c2ccc3cc4c(cc3c2)oc2ccc3oc5cc6cc(N(c7ccc8oc9ccccc9c8c7)c7ccc8oc9ccccc9c8c7)ccc6cc5c3c24)cc1 ; Cc1cc(N(c2cc(C)c(C)c(C)c2)c2ccc3cc4c(cc3c2)oc2ccc3oc5cc6cc(N(c7ccccc7)c7cccc8c7oc7ccccc78)ccc6cc5c3c24)cc(C)c1C ; CC(C)c1ccc(N(c2ccc(C(C)C)cc2)c2ccc3cc4c(cc3c2)oc2ccc3oc5cc6cc(N(c7ccccc7)c7cccc8c7oc7ccccc78)ccc6cc5c3c24)cc1]

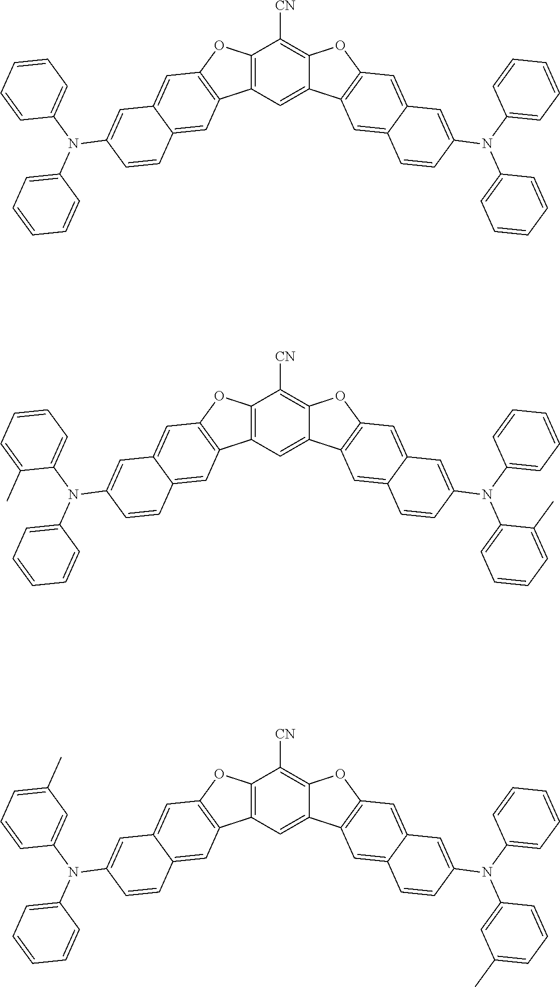 [SMILES: N#Cc1c2oc3cc4cc(N(c5ccccc5)c5ccccc5)ccc4cc3c2cc2c1oc1cc3cc(N(c4ccccc4)c4ccccc4)ccc3cc12 ; Cc1ccccc1N(c1ccccc1)c1ccc2cc3c(cc2c1)oc1c(C#N)c2oc4cc5cc(N(c6ccccc6)c6ccccc6C)ccc5cc4c2cc13 ; Cc1cccc(N(c2ccccc2)c2ccc3cc4c(cc3c2)oc2c(C#N)c3oc5cc6cc(N(c7ccccc7)c7cccc(C)c7)ccc6cc5c3cc24)c1]

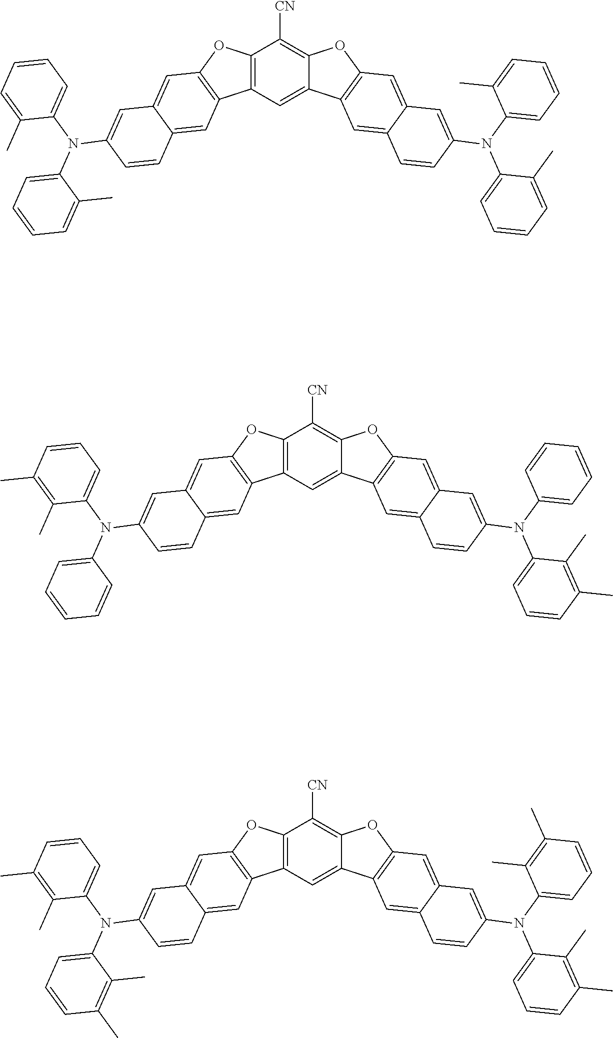

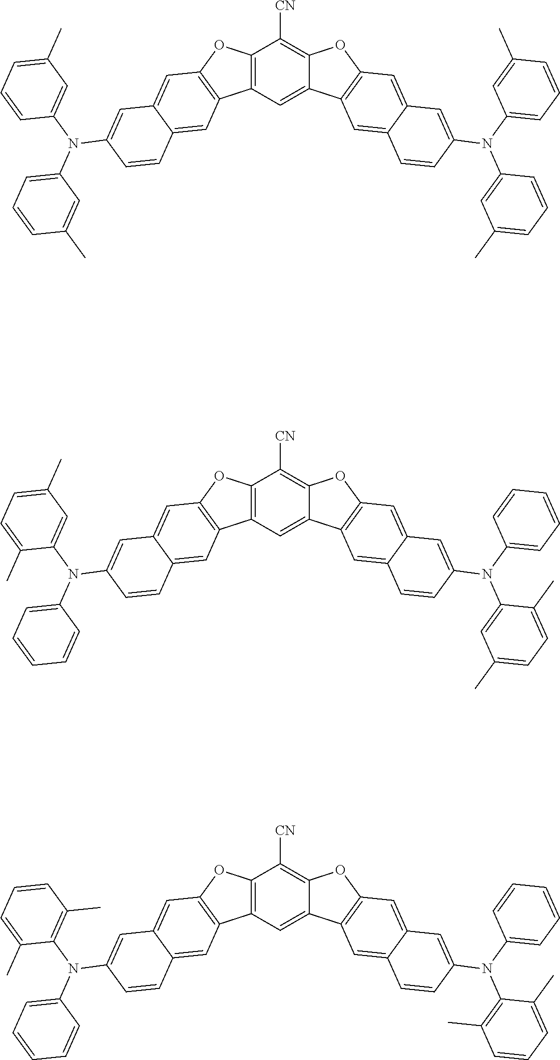 [SMILES: Cc1cccc(N(c2cccc(C)c2)c2ccc3cc4c(cc3c2)oc2c(C#N)c3oc5cc6cc(N(c7cccc(C)c7)c7cccc(C)c7)ccc6cc5c3cc24)c1 ; Cc1ccc(C)c(N(c2ccccc2)c2ccc3cc4c(cc3c2)oc2c(C#N)c3oc5cc6cc(N(c7ccccc7)c7cc(C)ccc7C)ccc6cc5c3cc24)c1 ; Cc1cccc(C)c1N(c1ccccc1)c1ccc2cc3c(cc2c1)oc1c(C#N)c2oc4cc5cc(N(c6ccccc6)c6c(C)cccc6C)ccc5cc4c2cc13]

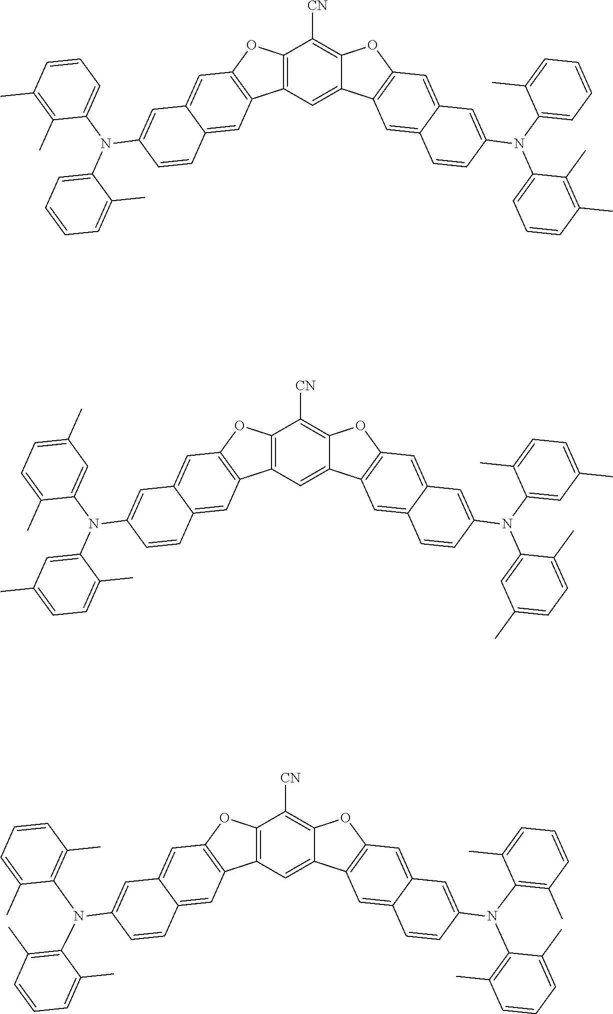

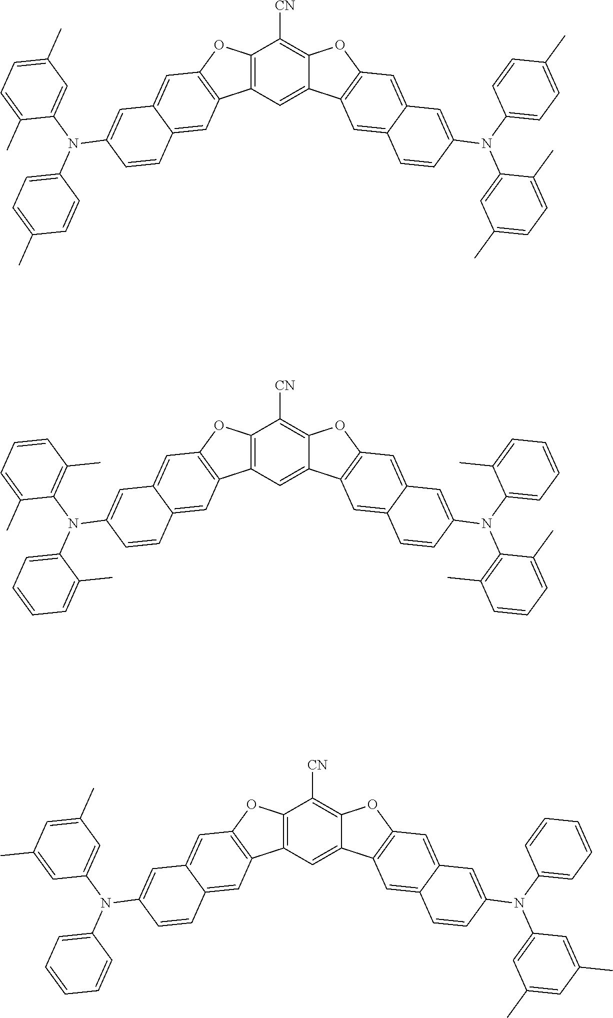

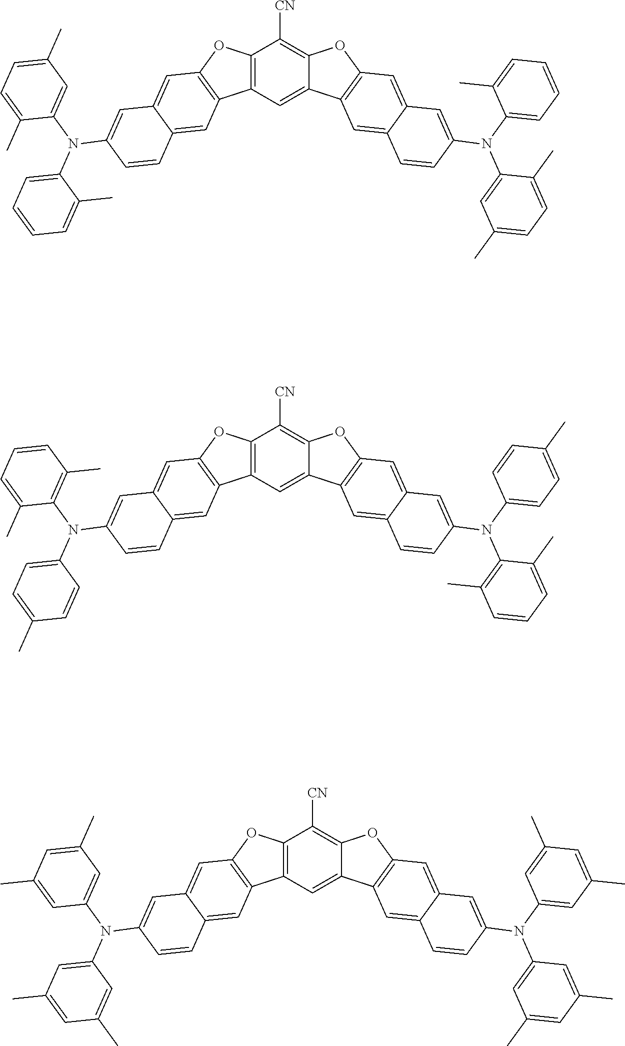

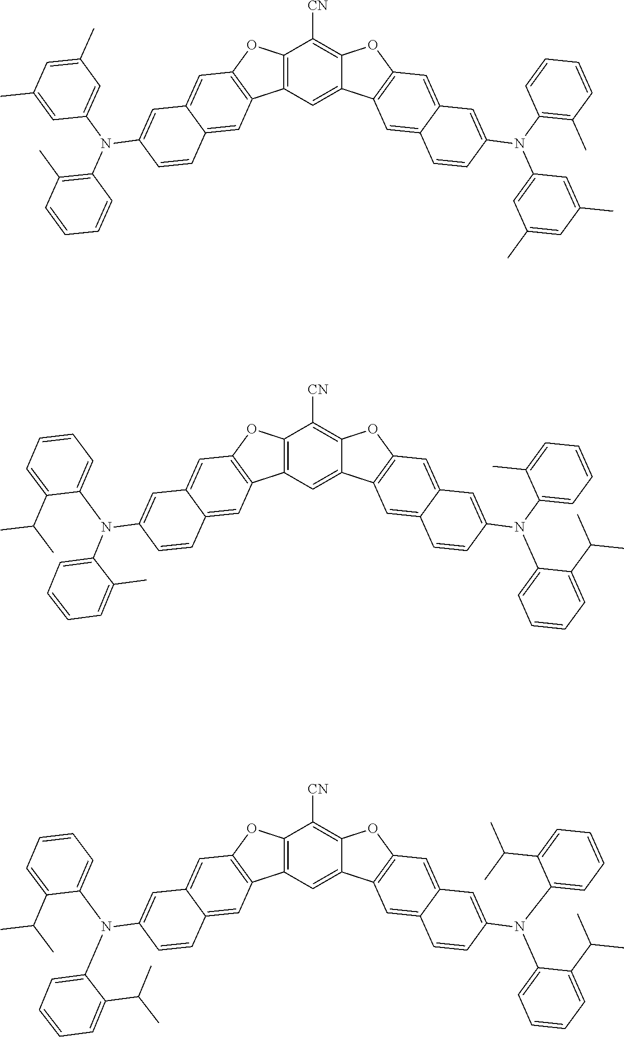

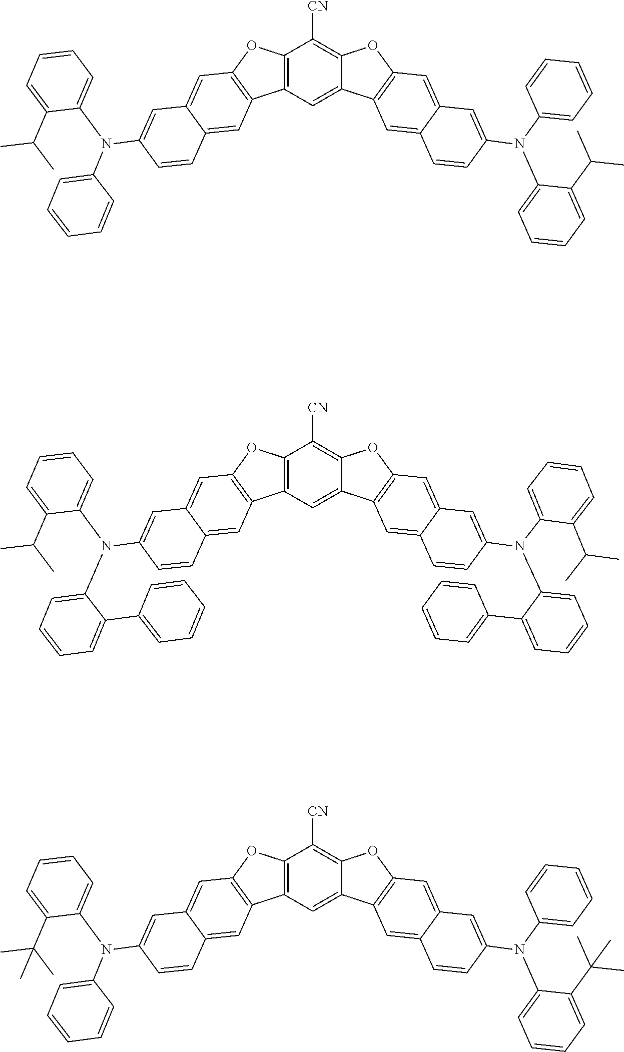

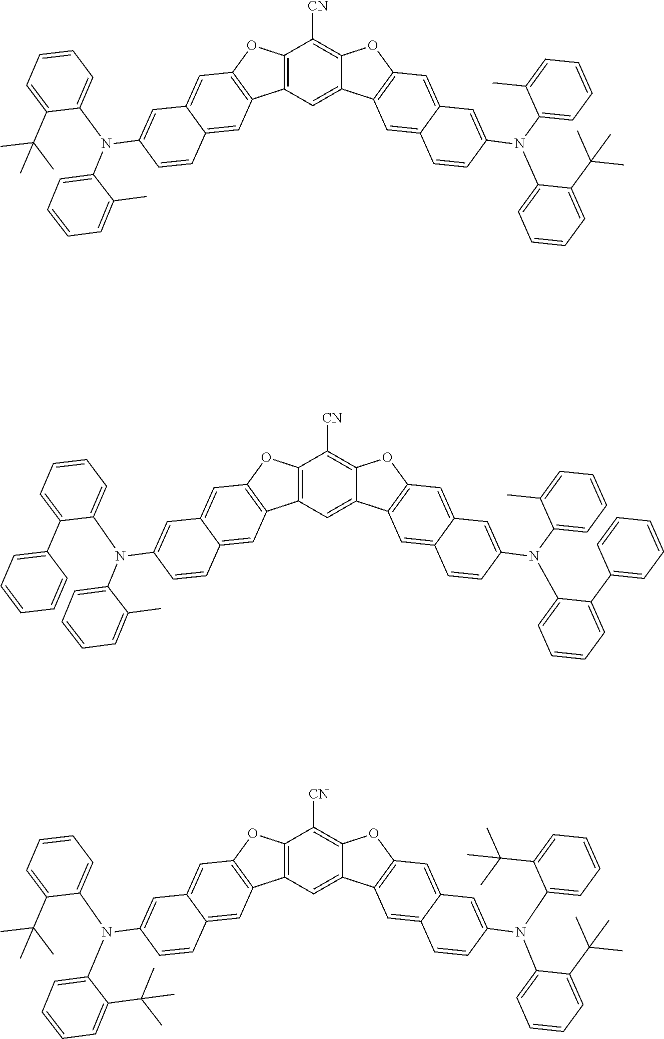

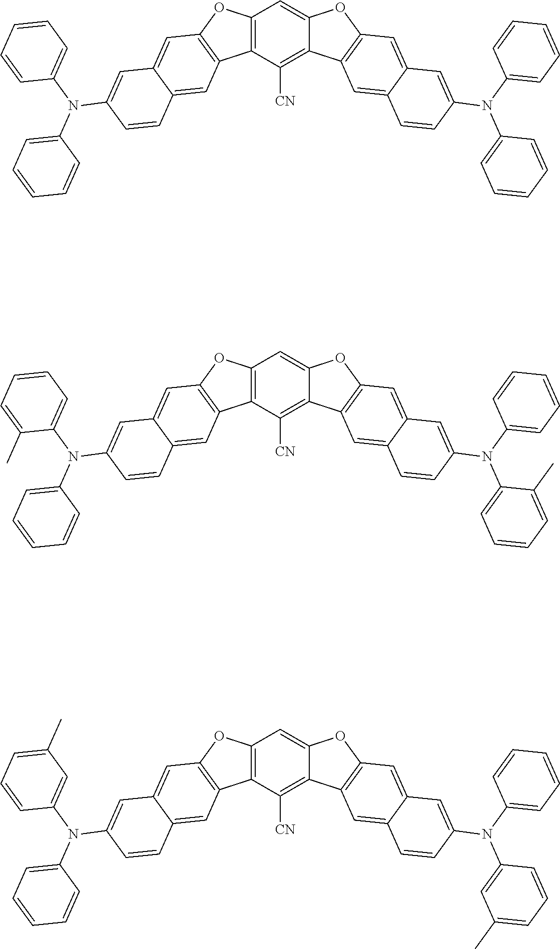 [SMILES: N#Cc1c2c(cc3oc4cc5cc(N(c6ccccc6)c6ccccc6)ccc5cc4c13)oc1cc3cc(N(c4ccccc4)c4ccccc4)ccc3cc12 ; Cc1ccccc1N(c1ccccc1)c1ccc2cc3c(cc2c1)oc1cc2oc4cc5cc(N(c6ccccc6)c6ccccc6C)ccc5cc4c2c(C#N)c13 ; Cc1cccc(N(c2ccccc2)c2ccc3cc4c(cc3c2)oc2cc3oc5cc6cc(N(c7ccccc7)c7cccc(C)c7)ccc6cc5c3c(C#N)c24)c1]

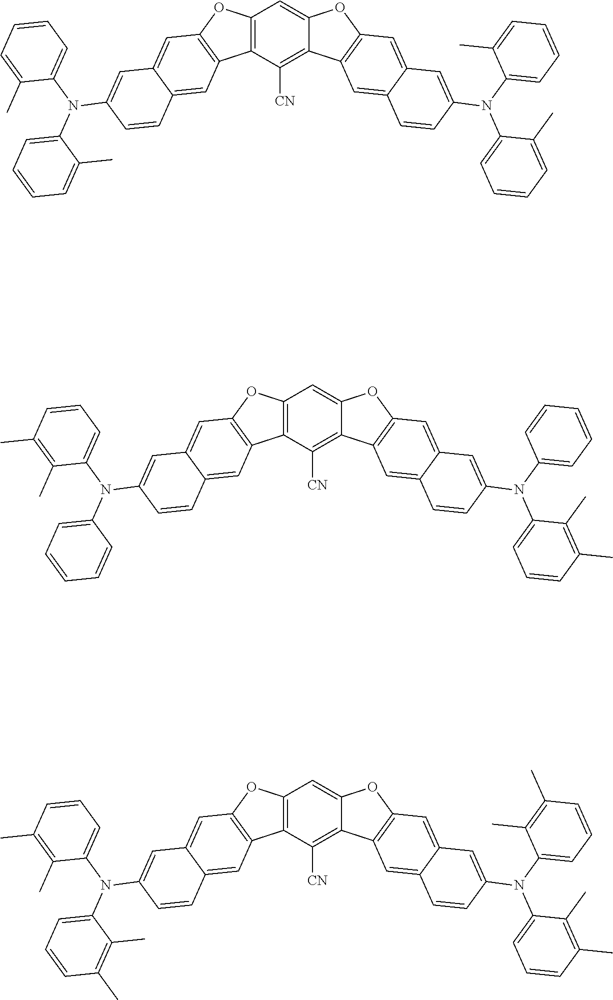

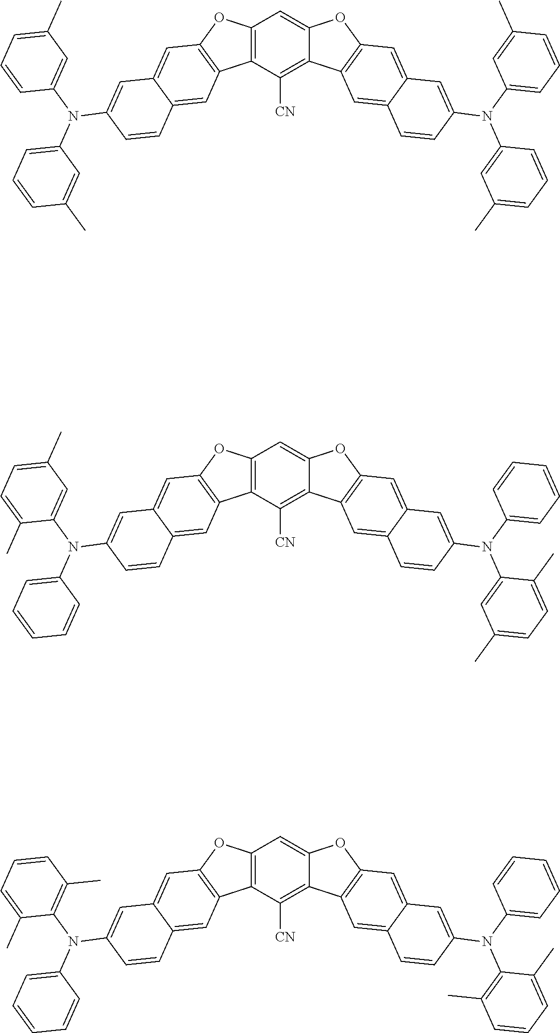 [SMILES: Cc1cccc(N(c2cccc(C)c2)c2ccc3cc4c(cc3c2)oc2cc3oc5cc6cc(N(c7cccc(C)c7)c7cccc(C)c7)ccc6cc5c3c(C#N)c24)c1 ; Cc1ccc(C)c(N(c2ccccc2)c2ccc3cc4c(cc3c2)oc2cc3oc5cc6cc(N(c7ccccc7)c7cc(C)ccc7C)ccc6cc5c3c(C#N)c24)c1 ; Cc1cccc(C)c1N(c1ccccc1)c1ccc2cc3c(cc2c1)oc1cc2oc4cc5cc(N(c6ccccc6)c6c(C)cccc6C)ccc5cc4c2c(C#N)c13]

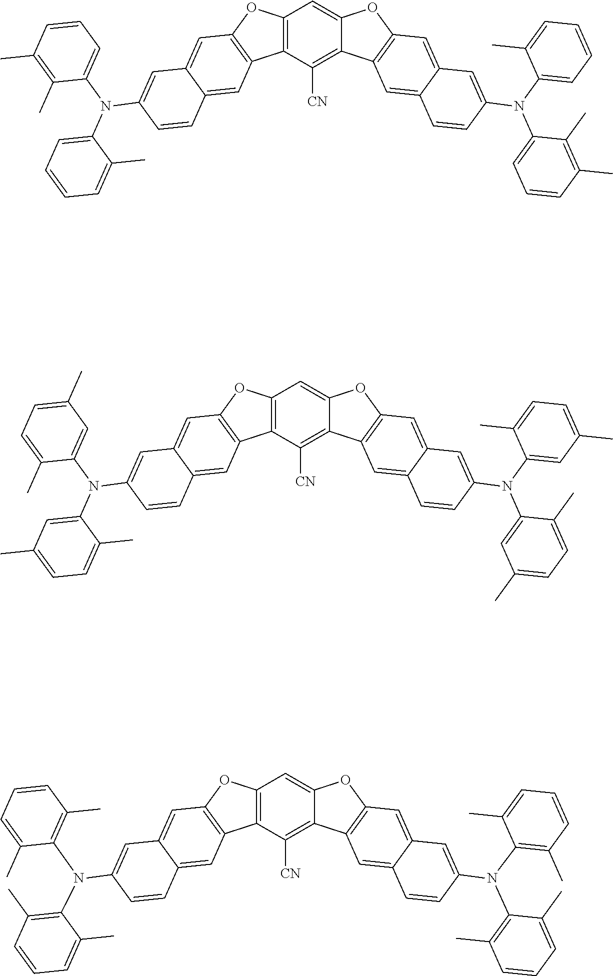

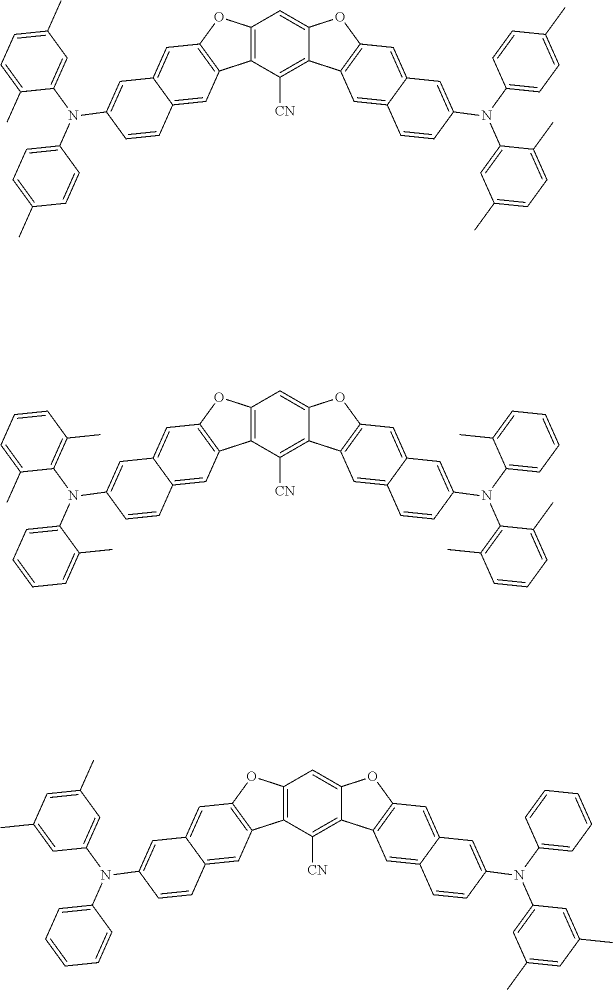

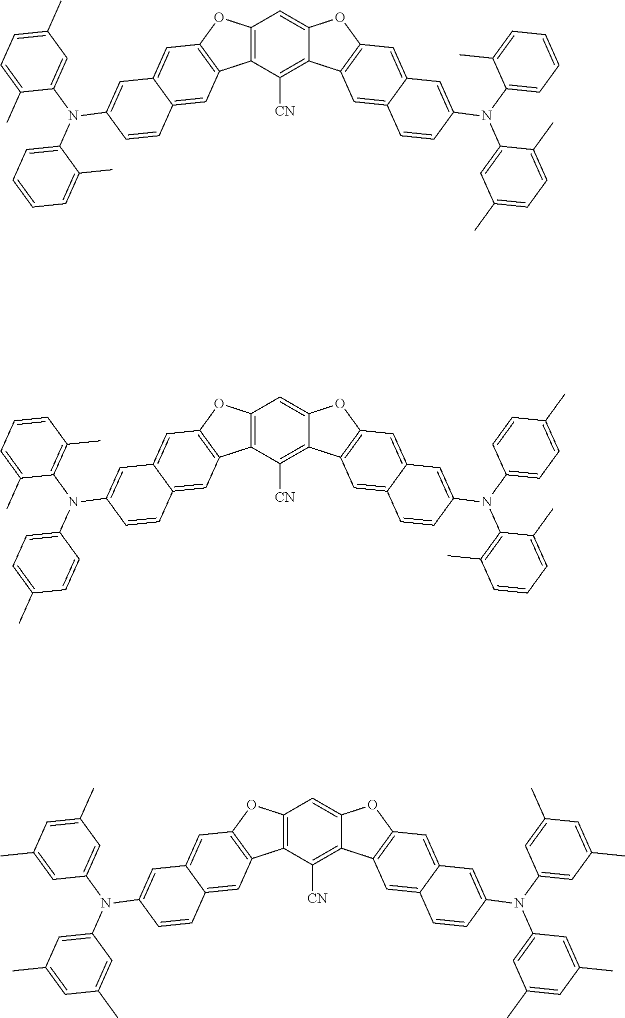

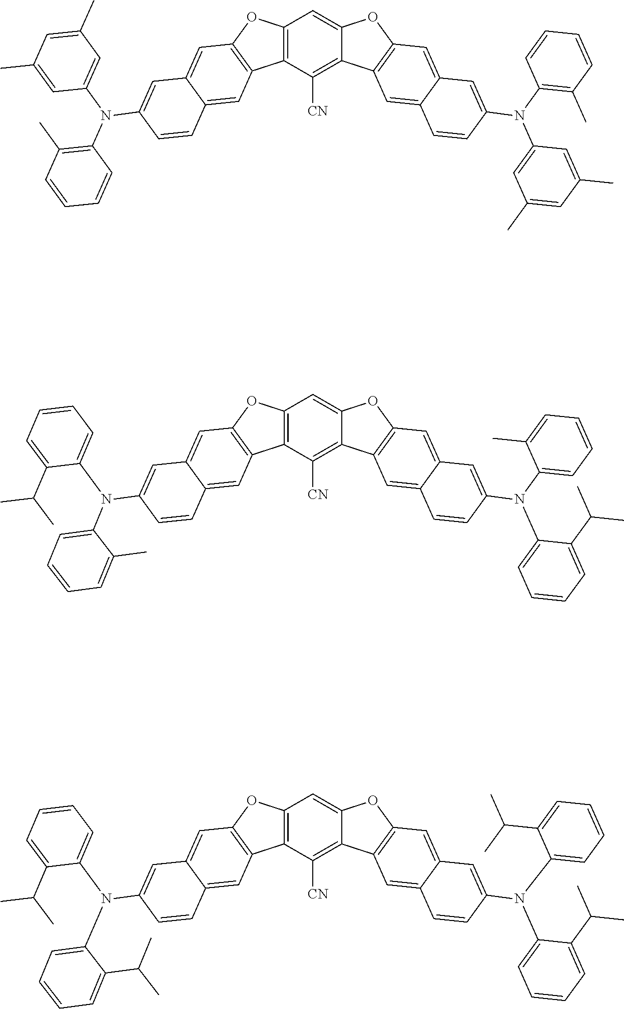

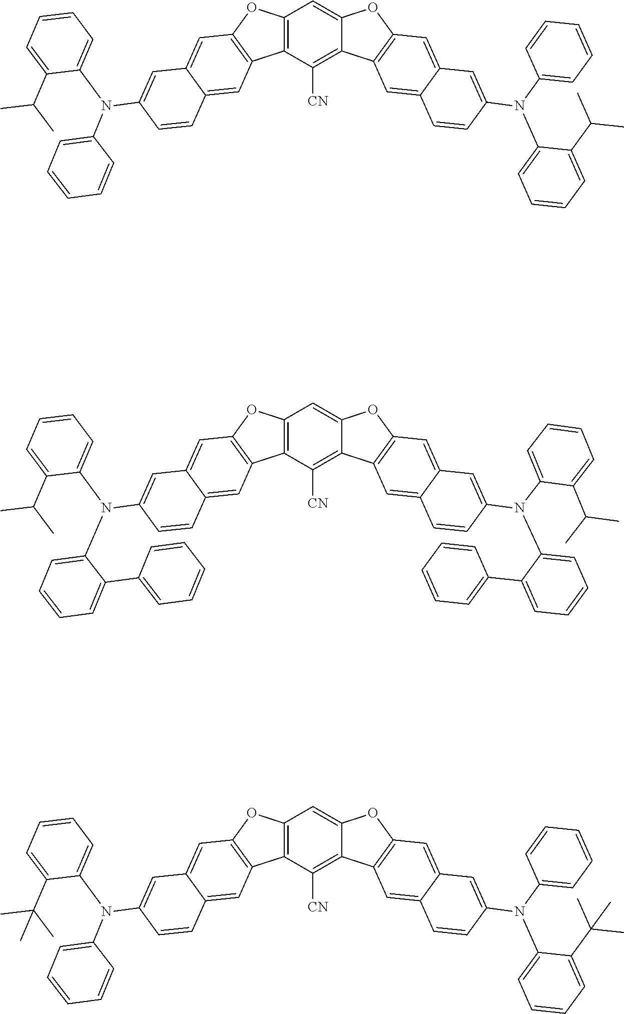

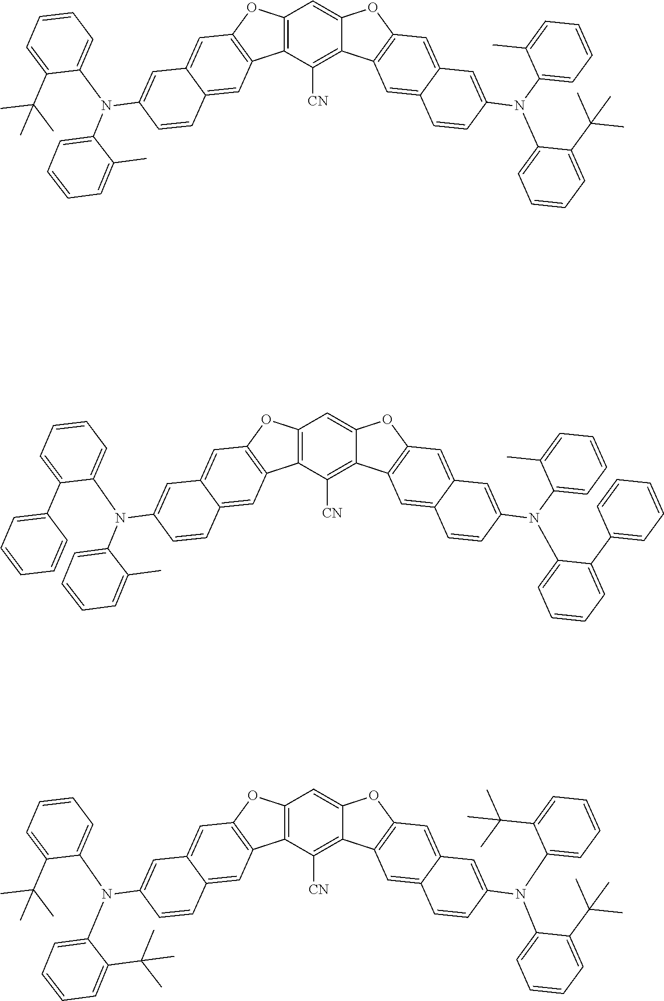

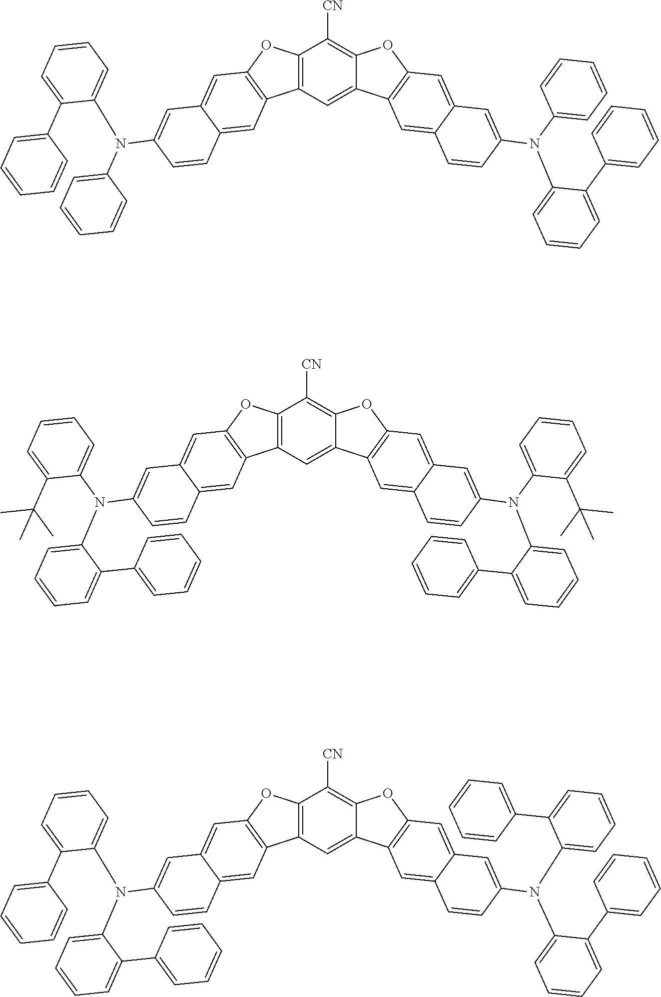 [SMILES: N#Cc1c2oc3cc4cc(N(c5ccccc5)c5ccccc5-c5ccccc5)ccc4cc3c2cc2c1oc1cc3cc(N(c4ccccc4)c4ccccc4-c4ccccc4)ccc3cc12 ; CC(C)(C)c1ccccc1N(c1ccc2cc3c(cc2c1)oc1c(C#N)c2oc4cc5cc(N(c6ccccc6-c6ccccc6)c6ccccc6C(C)(C)C)ccc5cc4c2cc13)c1ccccc1-c1ccccc1 ; N#Cc1c2oc3cc4cc(N(c5ccccc5-c5ccccc5)c5ccccc5-c5ccccc5)ccc4cc3c2cc2c1oc1cc3cc(N(c4ccccc4-c4ccccc4)c4ccccc4-c4ccccc4)ccc3cc12]

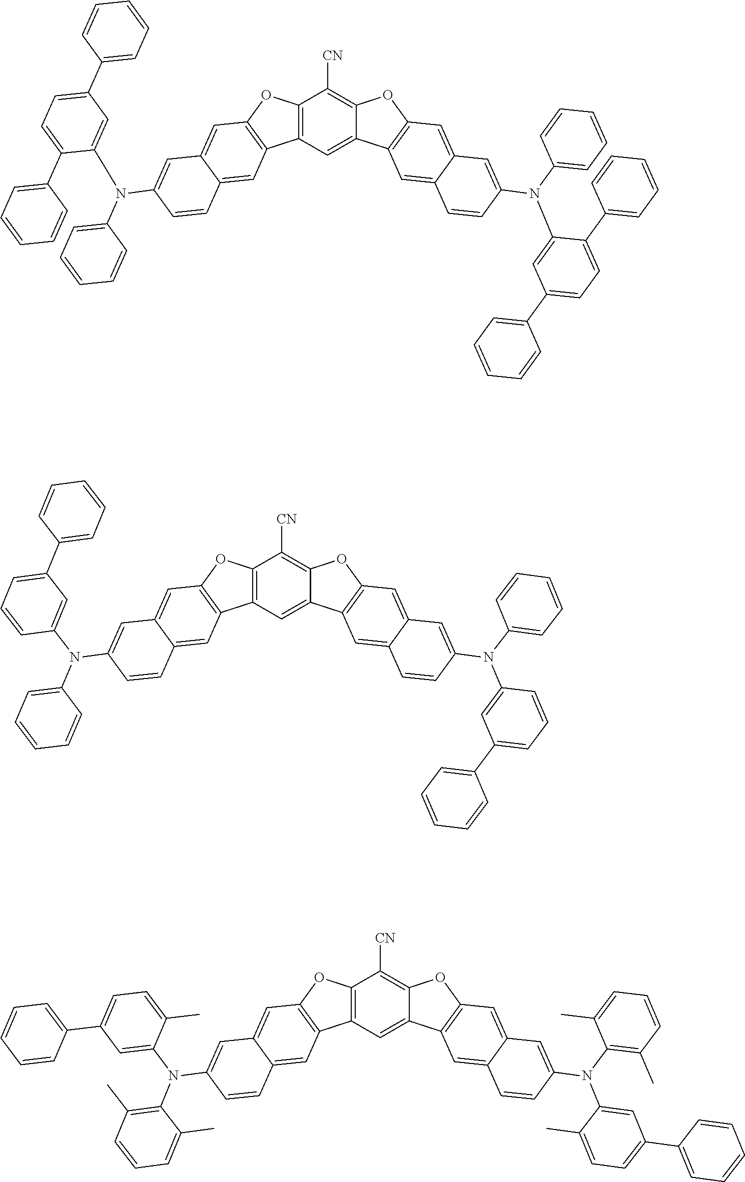 [SMILES: N#Cc1c2oc3cc4cc(N(c5ccccc5)c5cc(-c6ccccc6)ccc5-c5ccccc5)ccc4cc3c2cc2c1oc1cc3cc(N(c4ccccc4)c4cc(-c5ccccc5)ccc4-c4ccccc4)ccc3cc12 ; N#Cc1c2oc3cc4cc(N(c5ccccc5)c5cccc(-c6ccccc6)c5)ccc4cc3c2cc2c1oc1cc3cc(N(c4ccccc4)c4cccc(-c5ccccc5)c4)ccc3cc12 ; Cc1ccc(-c2ccccc2)cc1N(c1ccc2cc3c(cc2c1)oc1c(C#N)c2oc4cc5cc(N(c6cc(-c7ccccc7)ccc6C)c6c(C)cccc6C)ccc5cc4c2cc13)c1c(C)cccc1C]

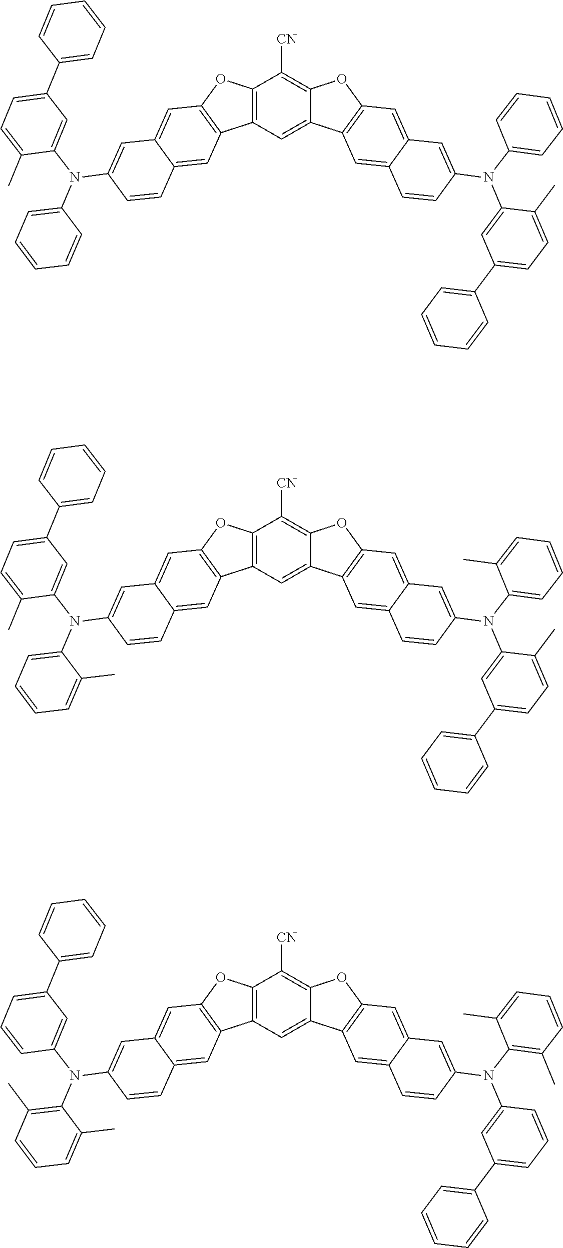 [SMILES: Cc1ccc(-c2ccccc2)cc1N(c1ccccc1)c1ccc2cc3c(cc2c1)oc1c(C#N)c2oc4cc5cc(N(c6ccccc6)c6cc(-c7ccccc7)ccc6C)ccc5cc4c2cc13 ; Cc1ccccc1N(c1ccc2cc3c(cc2c1)oc1c(C#N)c2oc4cc5cc(N(c6ccccc6C)c6cc(-c7ccccc7)ccc6C)ccc5cc4c2cc13)c1cc(-c2ccccc2)ccc1C ; Cc1cccc(C)c1N(c1cccc(-c2ccccc2)c1)c1ccc2cc3c(cc2c1)oc1c(C#N)c2oc4cc5cc(N(c6cccc(-c7ccccc7)c6)c6c(C)cccc6C)ccc5cc4c2cc13]

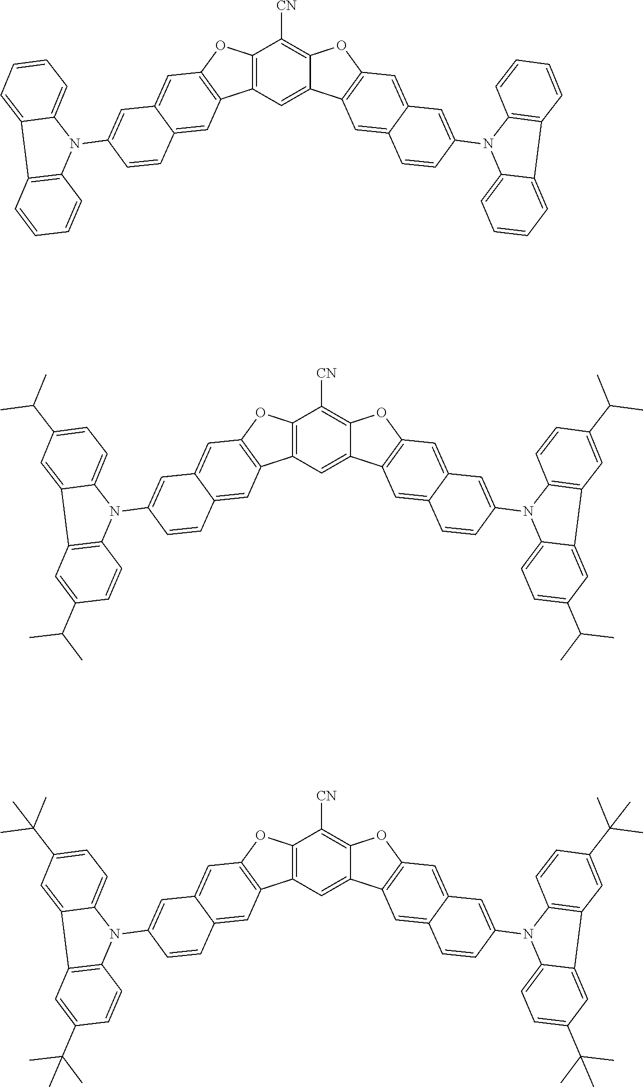

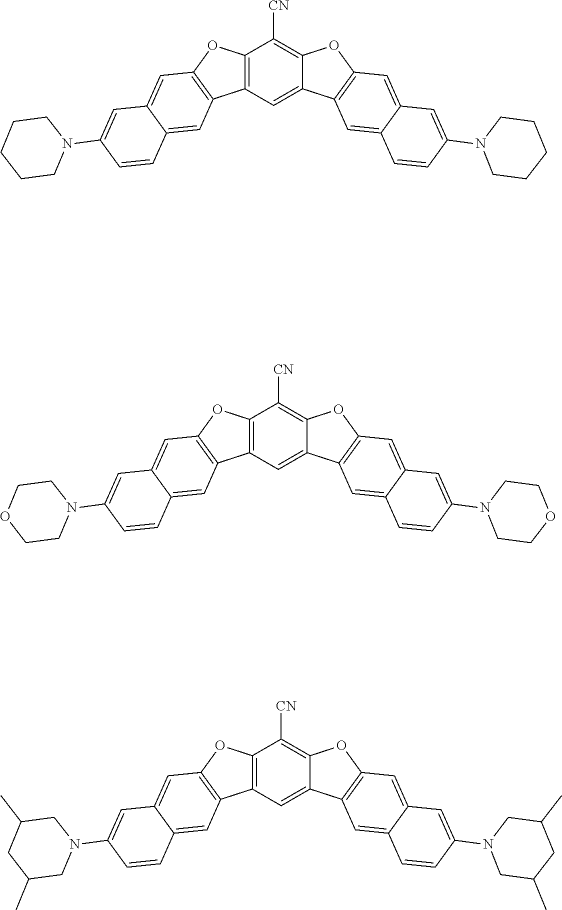

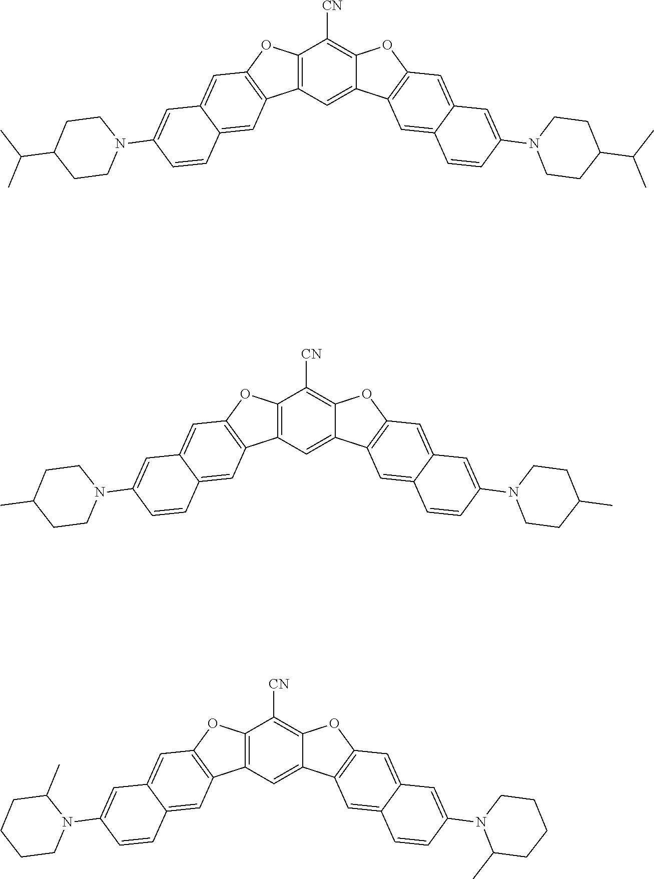

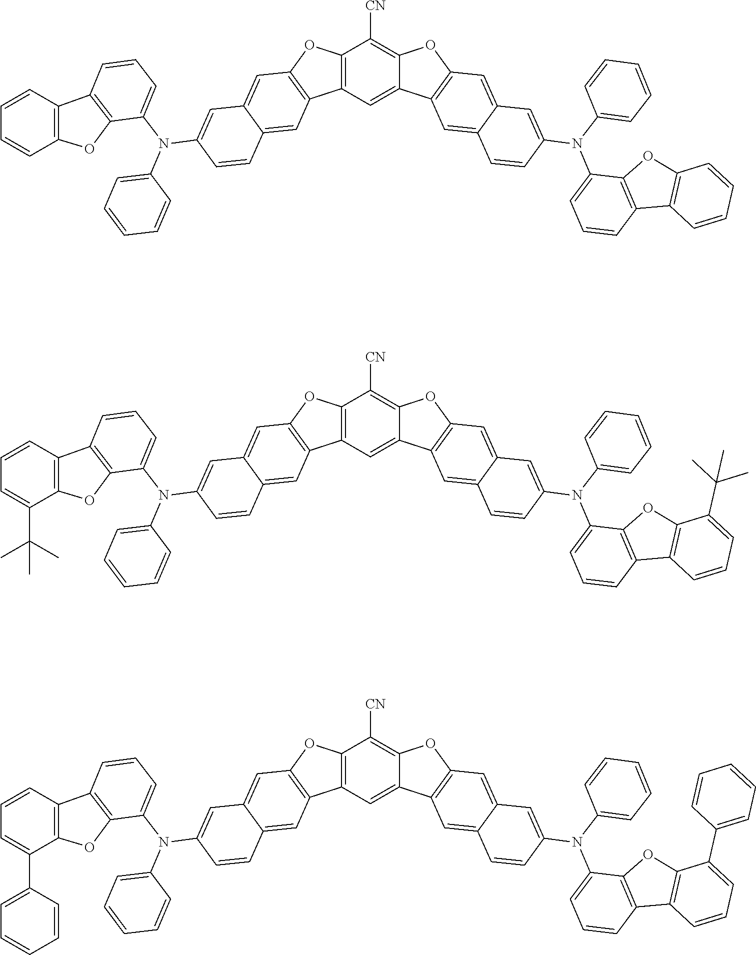 [SMILES: N#Cc1c2oc3cc4cc(N(c5ccccc5)c5cccc6c5oc5ccccc56)ccc4cc3c2cc2c1oc1cc3cc(N(c4ccccc4)c4cccc5c4oc4ccccc45)ccc3cc12 ; CC(C)(C)c1cccc2c1oc1c(N(c3ccccc3)c3ccc4cc5c(cc4c3)oc3c(C#N)c4oc6cc7cc(N(c8ccccc8)c8cccc9c8oc8c(C(C)(C)C)cccc89)ccc7cc6c4cc35)cccc12 ; N#Cc1c2oc3cc4cc(N(c5ccccc5)c5cccc6c5oc5c(-c7ccccc7)cccc56)ccc4cc3c2cc2c1oc1cc3cc(N(c4ccccc4)c4cccc5c4oc4c(-c6ccccc6)cccc45)ccc3cc12]

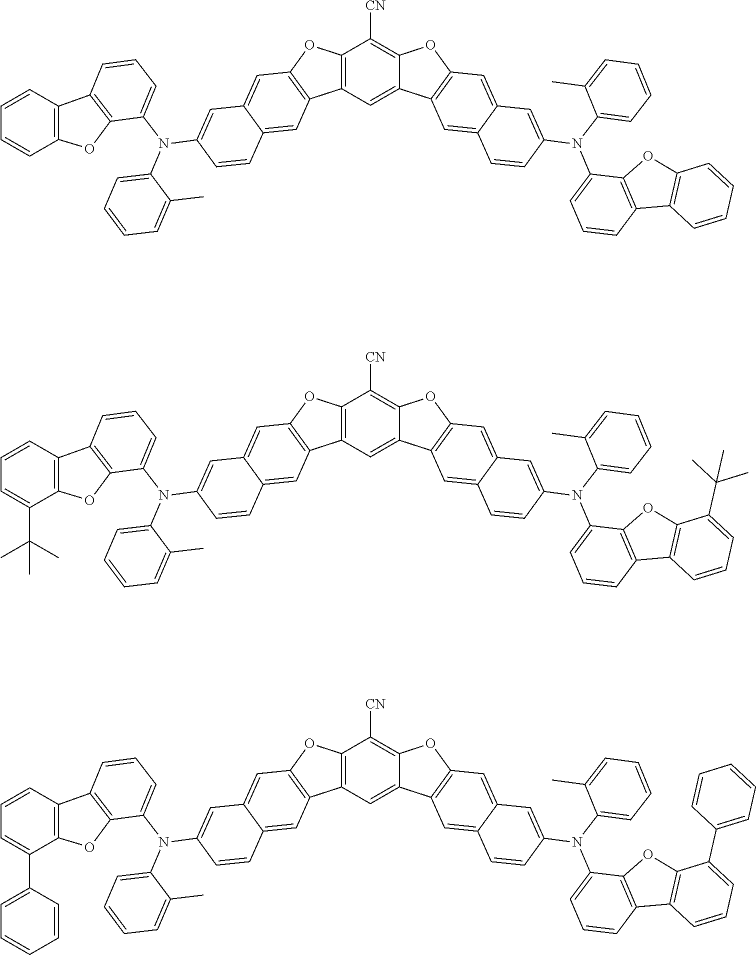 [SMILES: Cc1ccccc1N(c1ccc2cc3c(cc2c1)oc1c(C#N)c2oc4cc5cc(N(c6ccccc6C)c6cccc7c6oc6ccccc67)ccc5cc4c2cc13)c1cccc2c1oc1ccccc12 ; Cc1ccccc1N(c1ccc2cc3c(cc2c1)oc1c(C#N)c2oc4cc5cc(N(c6ccccc6C)c6cccc7c6oc6c(C(C)(C)C)cccc67)ccc5cc4c2cc13)c1cccc2c1oc1c(C(C)(C)C)cccc12 ; Cc1ccccc1N(c1ccc2cc3c(cc2c1)oc1c(C#N)c2oc4cc5cc(N(c6ccccc6C)c6cccc7c6oc6c(-c8ccccc8)cccc67)ccc5cc4c2cc13)c1cccc2c1oc1c(-c3ccccc3)cccc12]

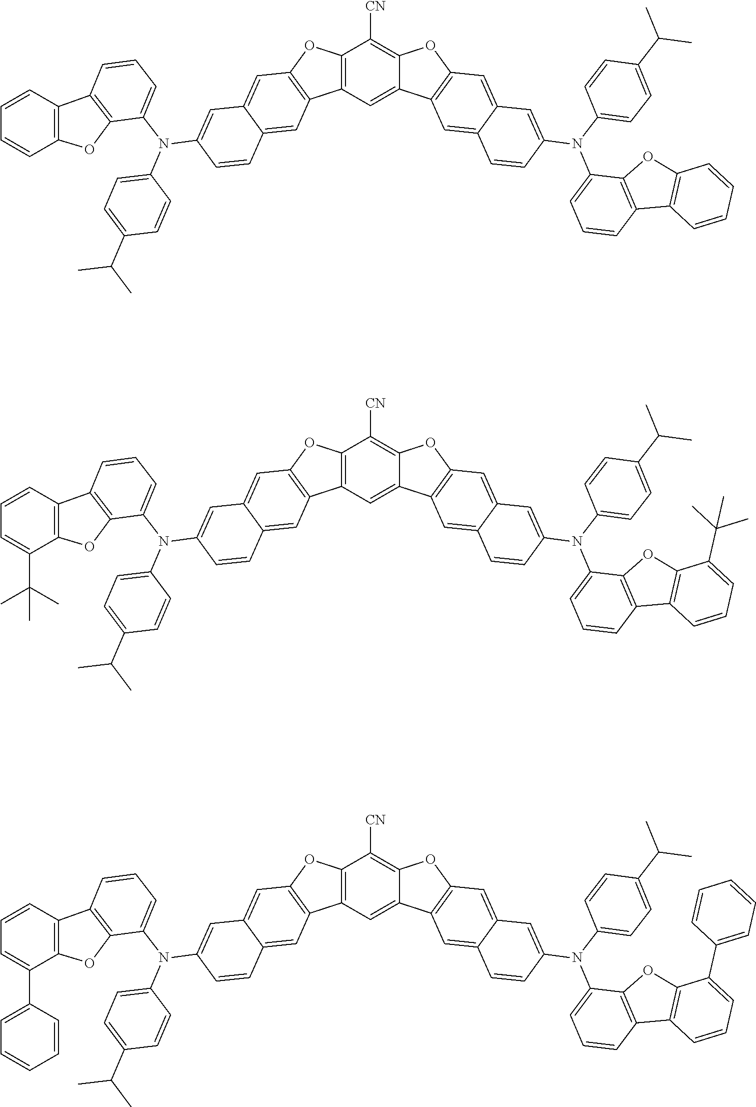 [SMILES: CC(C)c1ccc(N(c2ccc3cc4c(cc3c2)oc2c(C#N)c3oc5cc6cc(N(c7ccc(C(C)C)cc7)c7cccc8c7oc7ccccc78)ccc6cc5c3cc24)c2cccc3c2oc2ccccc23)cc1 ; CC(C)c1ccc(N(c2ccc3cc4c(cc3c2)oc2c(C#N)c3oc5cc6cc(N(c7ccc(C(C)C)cc7)c7cccc8c7oc7c(C(C)(C)C)cccc78)ccc6cc5c3cc24)c2cccc3c2oc2c(C(C)(C)C)cccc23)cc1 ; CC(C)c1ccc(N(c2ccc3cc4c(cc3c2)oc2c(C#N)c3oc5cc6cc(N(c7ccc(C(C)C)cc7)c7cccc8c7oc7c(-c9ccccc9)cccc78)ccc6cc5c3cc24)c2cccc3c2oc2c(-c4ccccc4)cccc23)cc1]

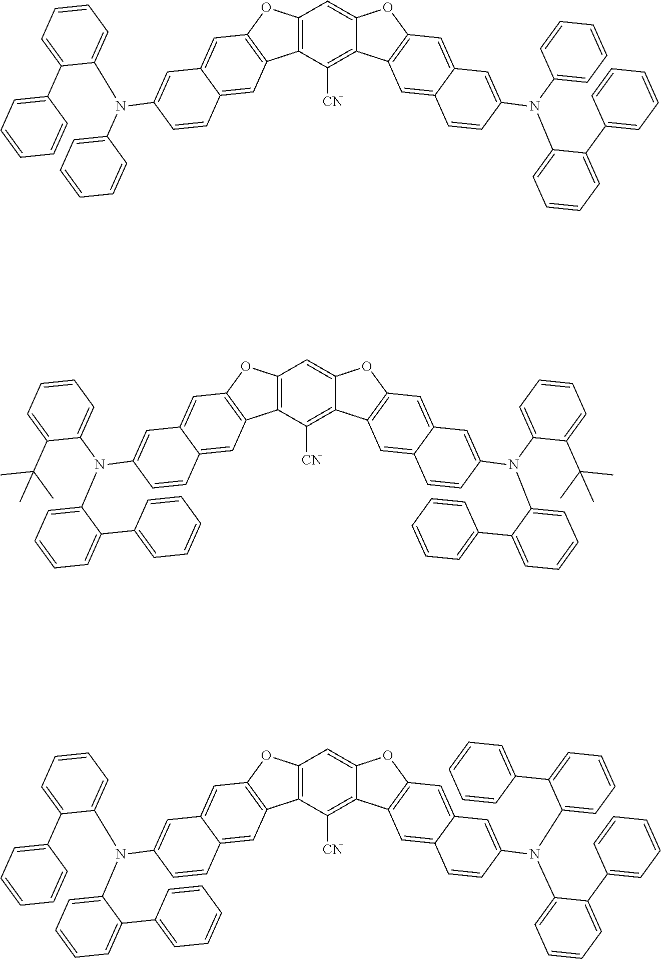 [SMILES: N#Cc1c2c(cc3oc4cc5cc(N(c6ccccc6)c6ccccc6-c6ccccc6)ccc5cc4c13)oc1cc3cc(N(c4ccccc4)c4ccccc4-c4ccccc4)ccc3cc12 ; CC(C)(C)c1ccccc1N(c1ccc2cc3c(cc2c1)oc1cc2oc4cc5cc(N(c6ccccc6-c6ccccc6)c6ccccc6C(C)(C)C)ccc5cc4c2c(C#N)c13)c1ccccc1-c1ccccc1 ; N#Cc1c2c(cc3oc4cc5cc(N(c6ccccc6-c6ccccc6)c6ccccc6-c6ccccc6)ccc5cc4c13)oc1cc3cc(N(c4ccccc4-c4ccccc4)c4ccccc4-c4ccccc4)ccc3cc12]

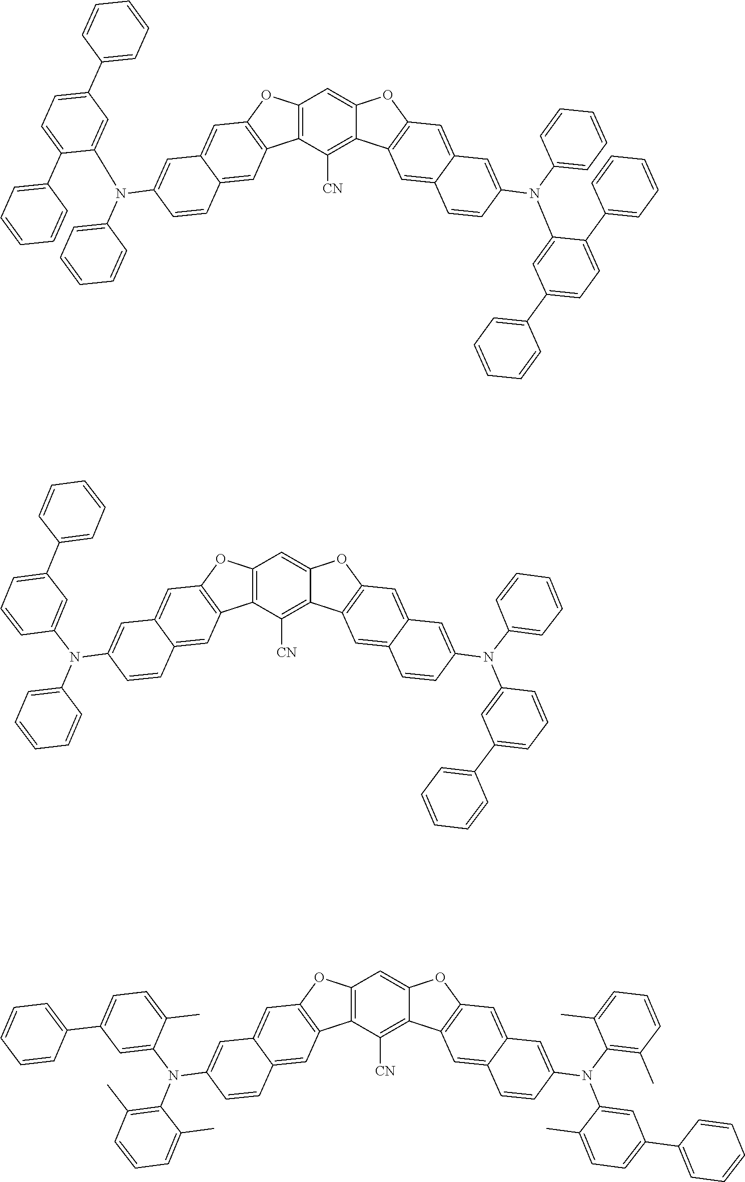 [SMILES: N#Cc1c2c(cc3oc4cc5cc(N(c6ccccc6)c6cc(-c7ccccc7)ccc6-c6ccccc6)ccc5cc4c13)oc1cc3cc(N(c4ccccc4)c4cc(-c5ccccc5)ccc4-c4ccccc4)ccc3cc12 ; N#Cc1c2c(cc3oc4cc5cc(N(c6ccccc6)c6cccc(-c7ccccc7)c6)ccc5cc4c13)oc1cc3cc(N(c4ccccc4)c4cccc(-c5ccccc5)c4)ccc3cc12 ; Cc1ccc(-c2ccccc2)cc1N(c1ccc2cc3c(cc2c1)oc1cc2oc4cc5cc(N(c6cc(-c7ccccc7)ccc6C)c6c(C)cccc6C)ccc5cc4c2c(C#N)c13)c1c(C)cccc1C]

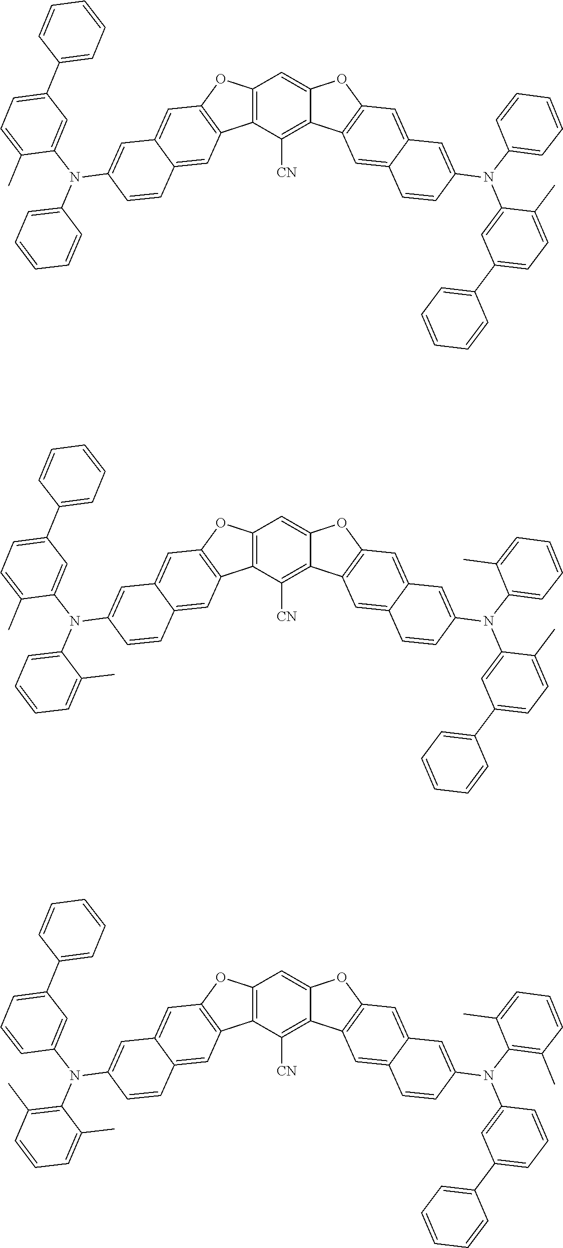 [SMILES: Cc1ccc(-c2ccccc2)cc1N(c1ccccc1)c1ccc2cc3c(cc2c1)oc1cc2oc4cc5cc(N(c6ccccc6)c6cc(-c7ccccc7)ccc6C)ccc5cc4c2c(C#N)c13 ; Cc1ccccc1N(c1ccc2cc3c(cc2c1)oc1cc2oc4cc5cc(N(c6ccccc6C)c6cc(-c7ccccc7)ccc6C)ccc5cc4c2c(C#N)c13)c1cc(-c2ccccc2)ccc1C ; Cc1cccc(C)c1N(c1cccc(-c2ccccc2)c1)c1ccc2cc3c(cc2c1)oc1cc2oc4cc5cc(N(c6cccc(-c7ccccc7)c6)c6c(C)cccc6C)ccc5cc4c2c(C#N)c13]

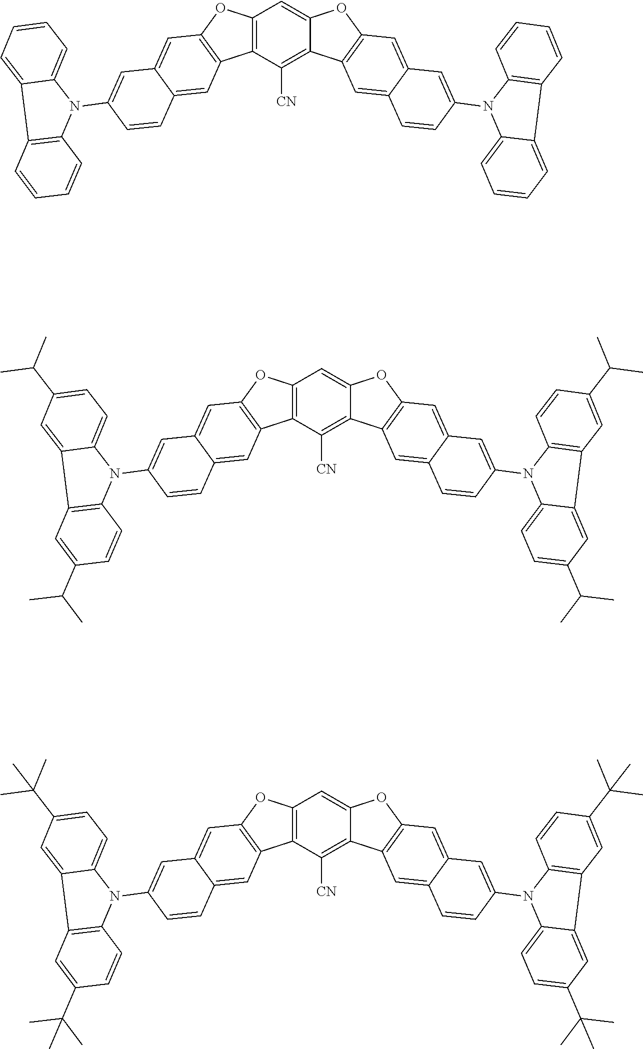

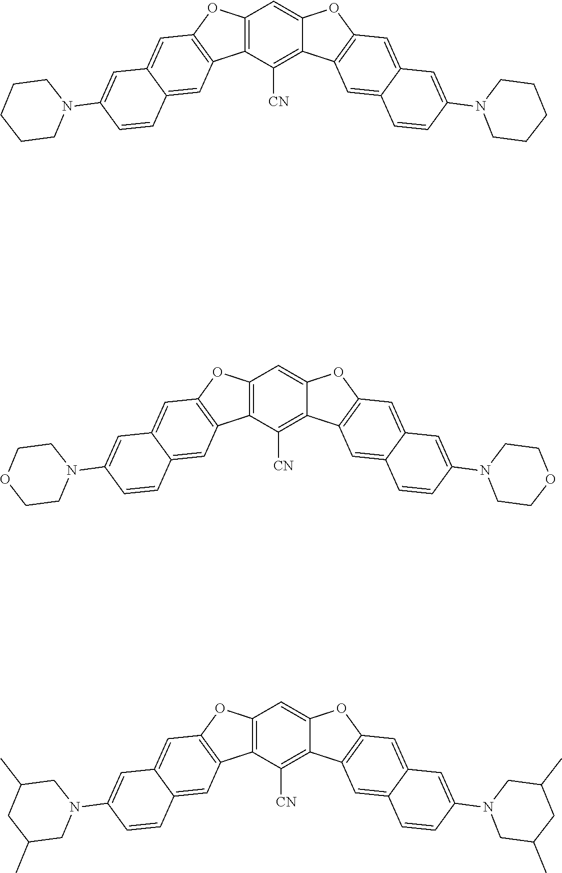

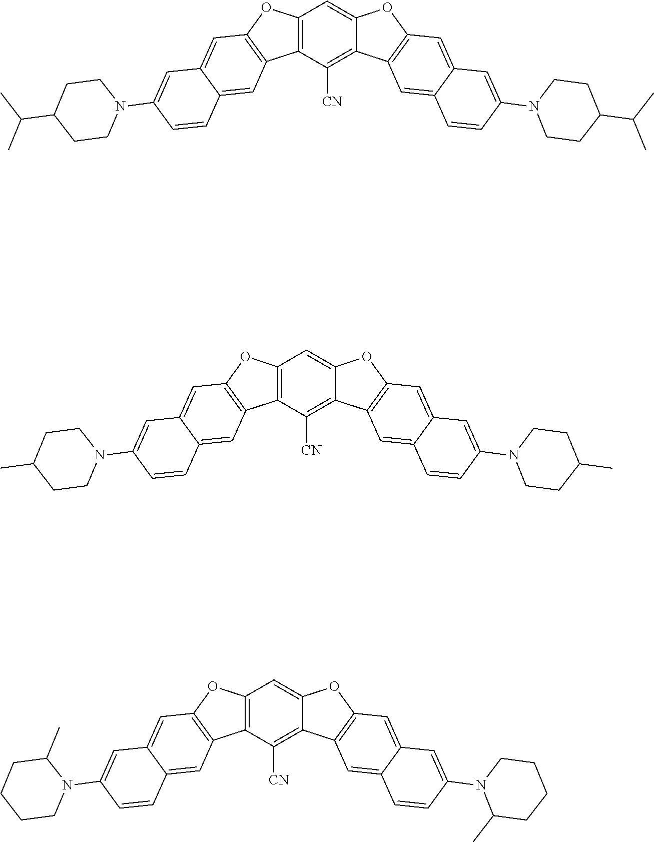

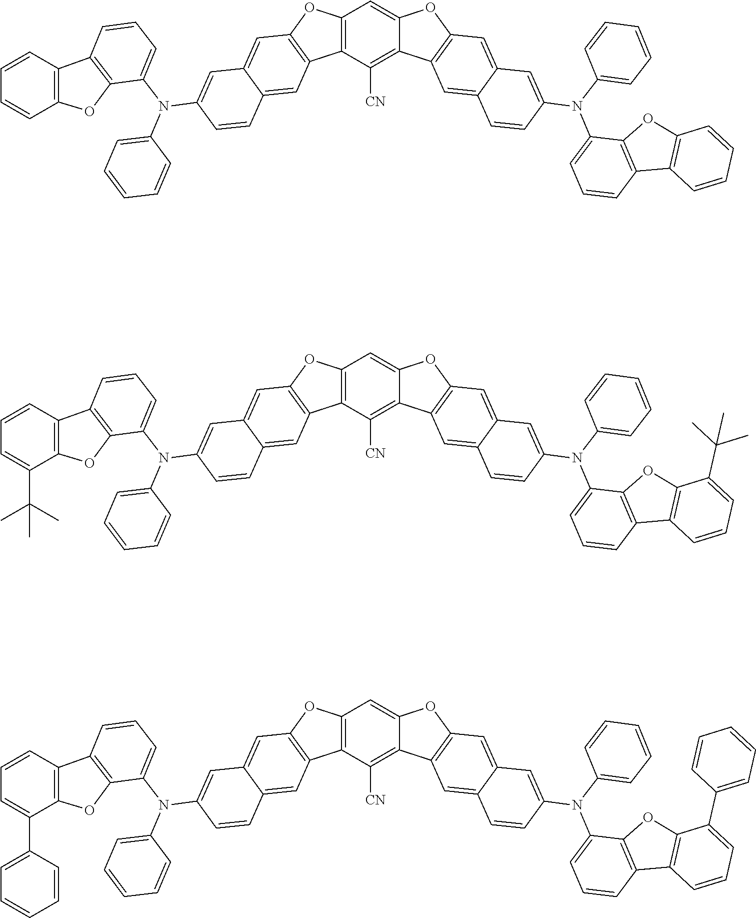 [SMILES: N#Cc1c2c(cc3oc4cc5cc(N(c6ccccc6)c6cccc7c6oc6ccccc67)ccc5cc4c13)oc1cc3cc(N(c4ccccc4)c4cccc5c4oc4ccccc45)ccc3cc12 ; CC(C)(C)c1cccc2c1oc1c(N(c3ccccc3)c3ccc4cc5c(cc4c3)oc3cc4oc6cc7cc(N(c8ccccc8)c8cccc9c8oc8c(C(C)(C)C)cccc89)ccc7cc6c4c(C#N)c35)cccc12 ; N#Cc1c2c(cc3oc4cc5cc(N(c6ccccc6)c6cccc7c6oc6c(-c8ccccc8)cccc67)ccc5cc4c13)oc1cc3cc(N(c4ccccc4)c4cccc5c4oc4c(-c6ccccc6)cccc45)ccc3cc12]

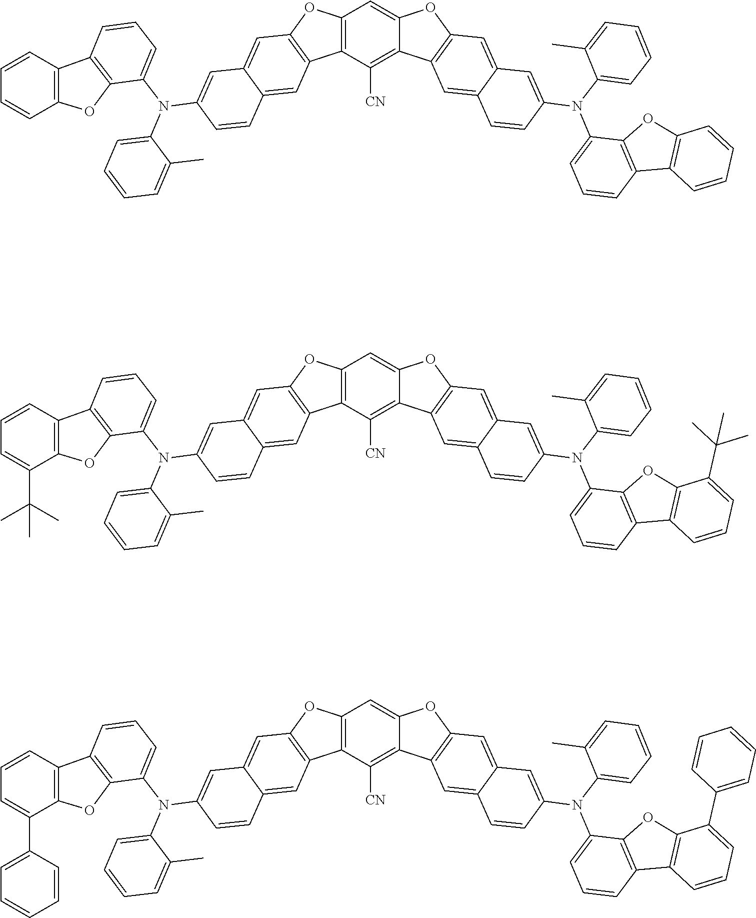 [SMILES: Cc1ccccc1N(c1ccc2cc3c(cc2c1)oc1cc2oc4cc5cc(N(c6ccccc6C)c6cccc7c6oc6ccccc67)ccc5cc4c2c(C#N)c13)c1cccc2c1oc1ccccc12 ; Cc1ccccc1N(c1ccc2cc3c(cc2c1)oc1cc2oc4cc5cc(N(c6ccccc6C)c6cccc7c6oc6c(C(C)(C)C)cccc67)ccc5cc4c2c(C#N)c13)c1cccc2c1oc1c(C(C)(C)C)cccc12 ; Cc1ccccc1N(c1ccc2cc3c(cc2c1)oc1cc2oc4cc5cc(N(c6ccccc6C)c6cccc7c6oc6c(-c8ccccc8)cccc67)ccc5cc4c2c(C#N)c13)c1cccc2c1oc1c(-c3ccccc3)cccc12]

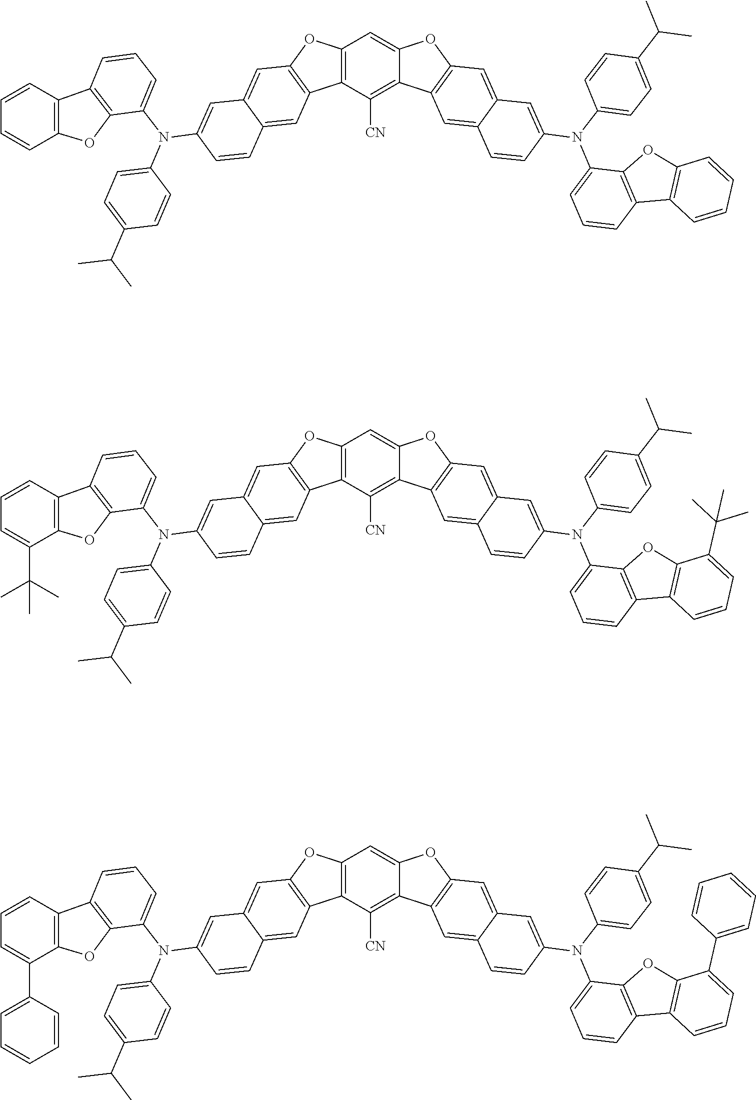 [SMILES: CC(C)c1ccc(N(c2ccc3cc4c(cc3c2)oc2cc3oc5cc6cc(N(c7ccc(C(C)C)cc7)c7cccc8c7oc7ccccc78)ccc6cc5c3c(C#N)c24)c2cccc3c2oc2ccccc23)cc1 ; CC(C)c1ccc(N(c2ccc3cc4c(cc3c2)oc2cc3oc5cc6cc(N(c7ccc(C(C)C)cc7)c7cccc8c7oc7c(C(C)(C)C)cccc78)ccc6cc5c3c(C#N)c24)c2cccc3c2oc2c(C(C)(C)C)cccc23)cc1 ; CC(C)c1ccc(N(c2ccc3cc4c(cc3c2)oc2cc3oc5cc6cc(N(c7ccc(C(C)C)cc7)c7cccc8c7oc7c(-c9ccccc9)cccc78)ccc6cc5c3c(C#N)c24)c2cccc3c2oc2c(-c4ccccc4)cccc23)cc1]

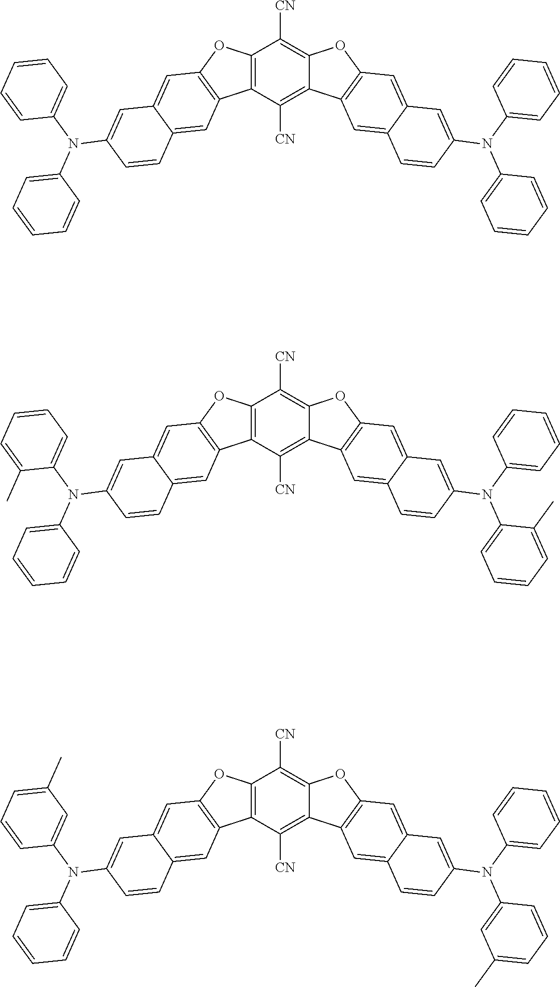 [SMILES: N#Cc1c2oc3cc4cc(N(c5ccccc5)c5ccccc5)ccc4cc3c2c(C#N)c2c1oc1cc3cc(N(c4ccccc4)c4ccccc4)ccc3cc12 ; Cc1ccccc1N(c1ccccc1)c1ccc2cc3c(cc2c1)oc1c(C#N)c2oc4cc5cc(N(c6ccccc6)c6ccccc6C)ccc5cc4c2c(C#N)c13 ; Cc1cccc(N(c2ccccc2)c2ccc3cc4c(cc3c2)oc2c(C#N)c3oc5cc6cc(N(c7ccccc7)c7cccc(C)c7)ccc6cc5c3c(C#N)c24)c1]

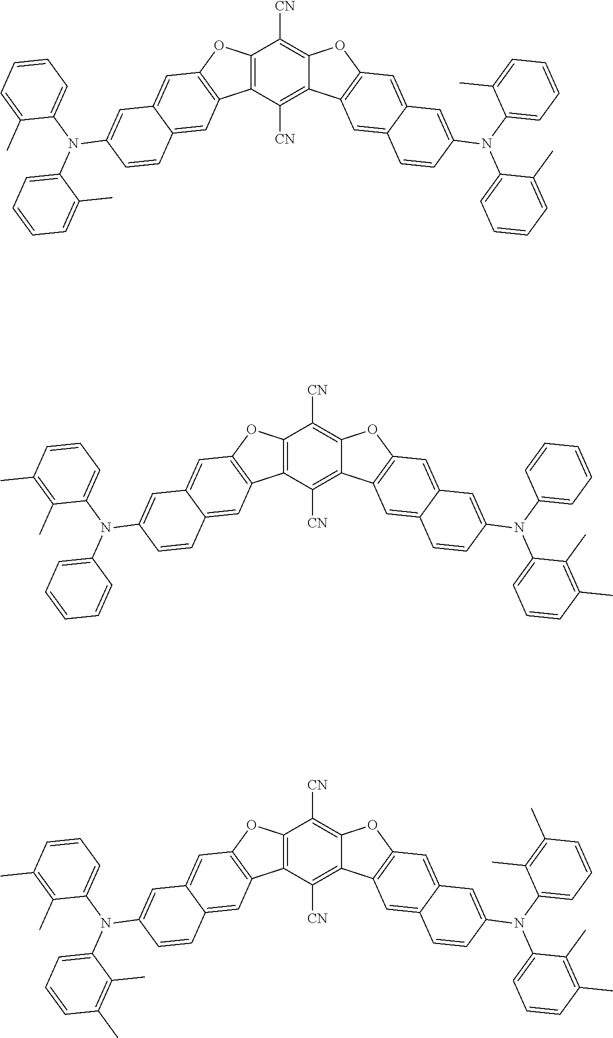

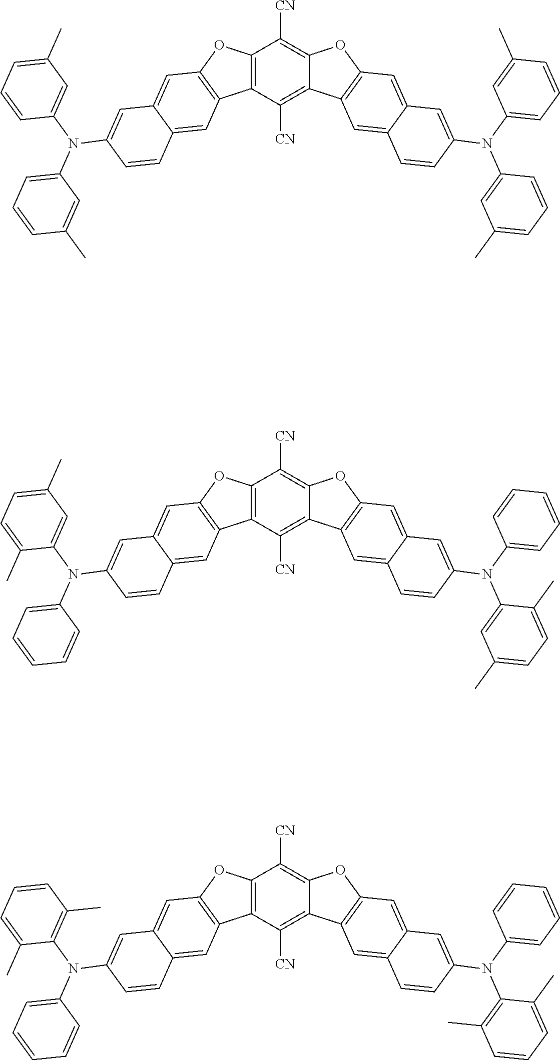 [SMILES: Cc1cccc(N(c2cccc(C)c2)c2ccc3cc4c(cc3c2)oc2c(C#N)c3oc5cc6cc(N(c7cccc(C)c7)c7cccc(C)c7)ccc6cc5c3c(C#N)c24)c1 ; Cc1ccc(C)c(N(c2ccccc2)c2ccc3cc4c(cc3c2)oc2c(C#N)c3oc5cc6cc(N(c7ccccc7)c7cc(C)ccc7C)ccc6cc5c3c(C#N)c24)c1 ; Cc1cccc(C)c1N(c1ccccc1)c1ccc2cc3c(cc2c1)oc1c(C#N)c2oc4cc5cc(N(c6ccccc6)c6c(C)cccc6C)ccc5cc4c2c(C#N)c13]

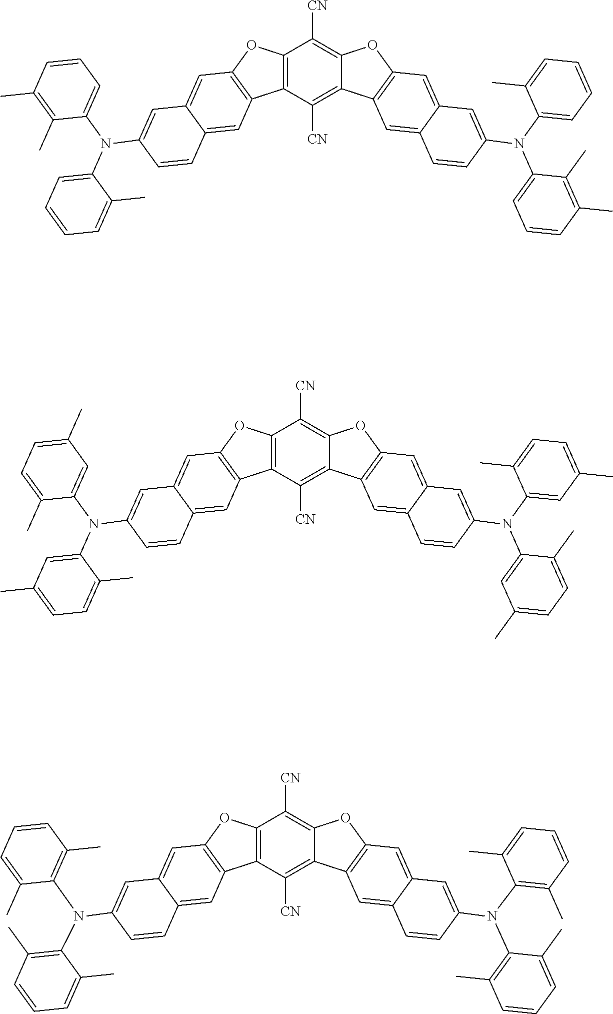

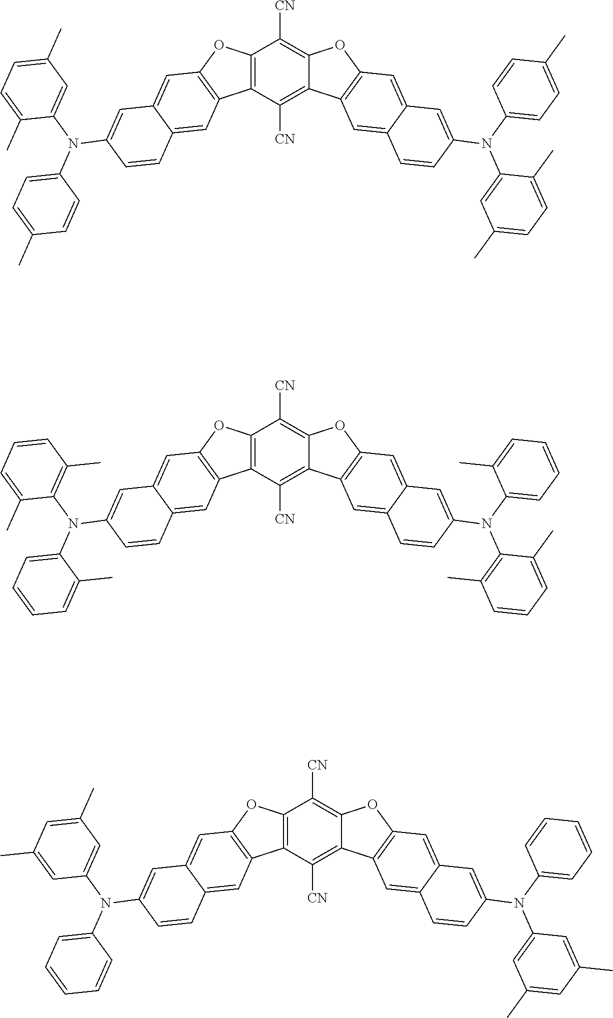

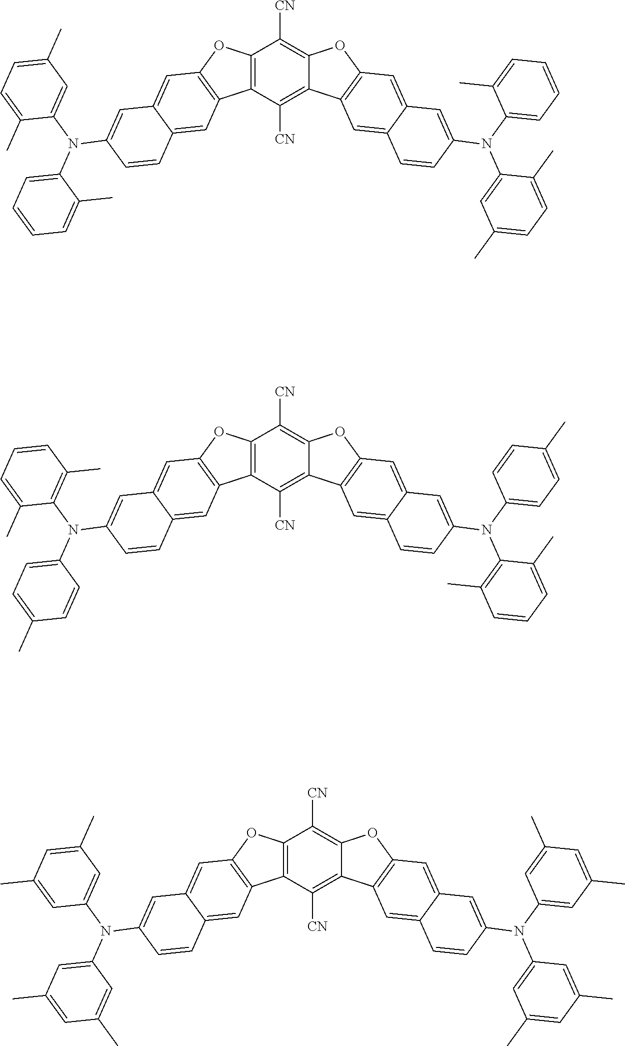

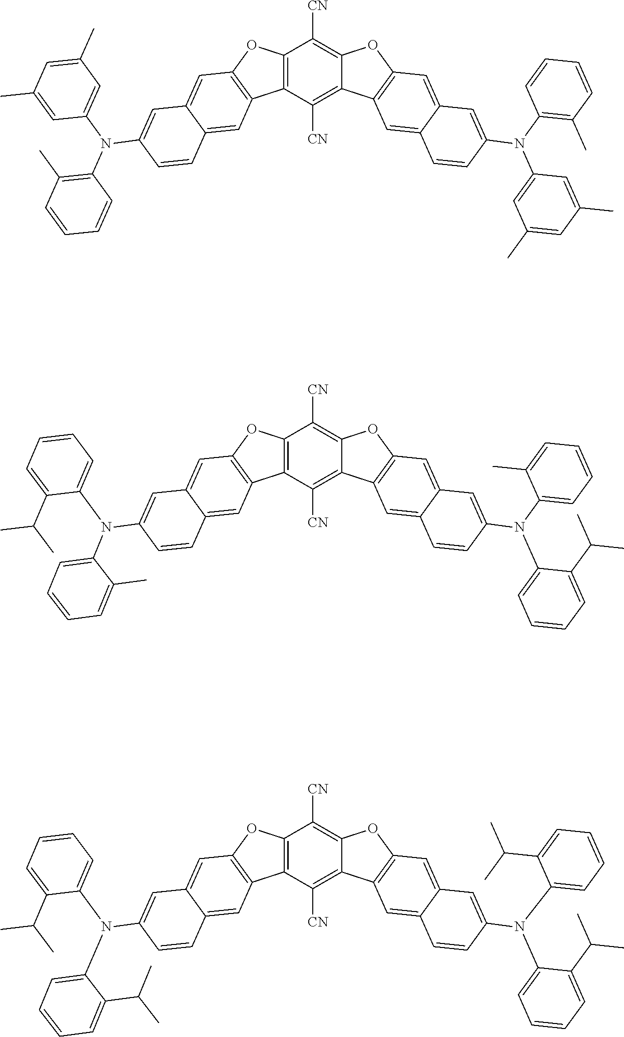

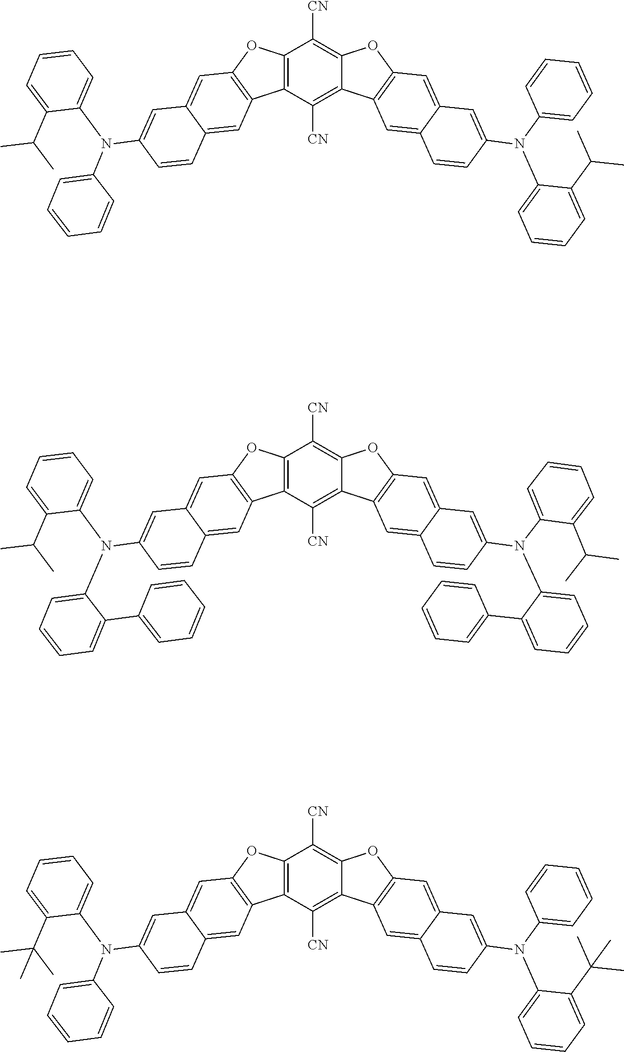

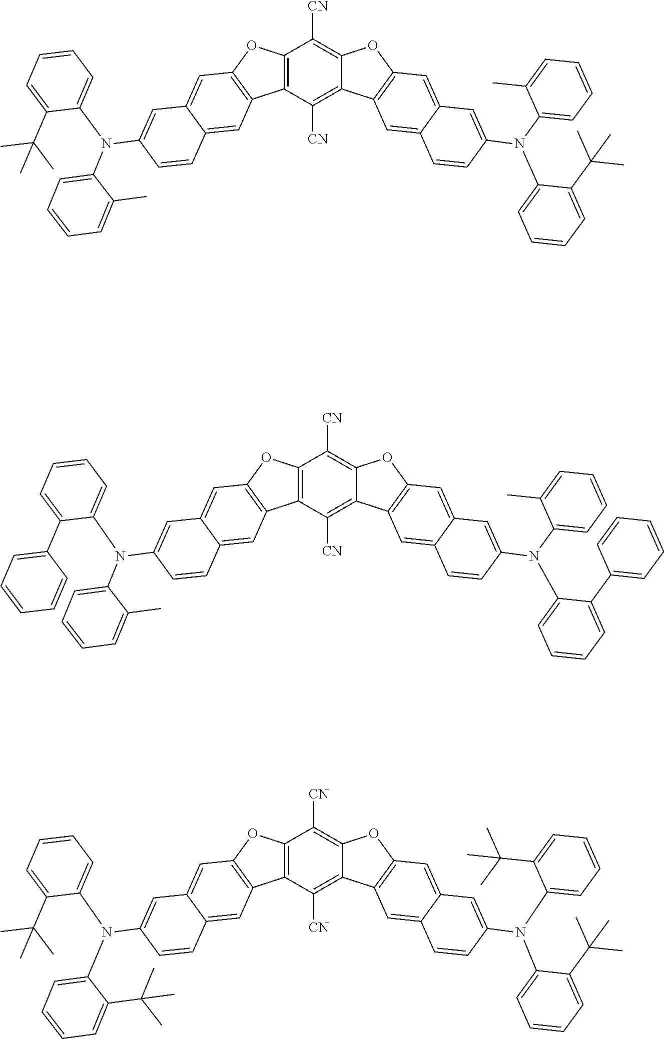

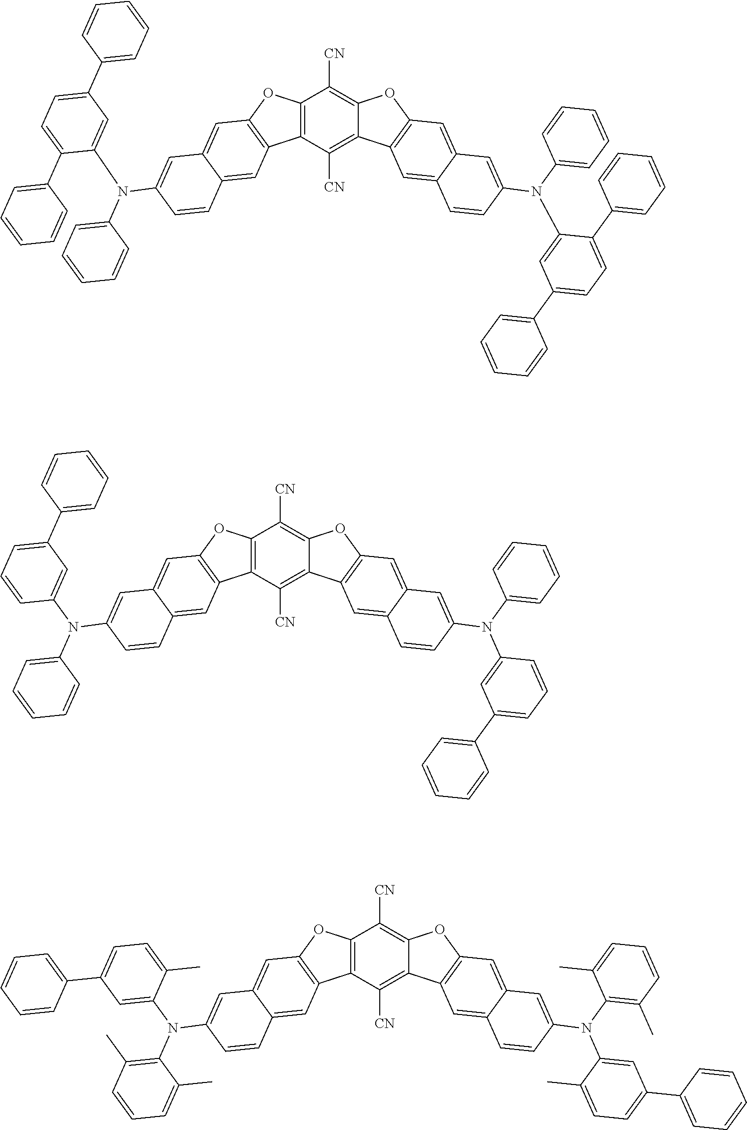 [SMILES: N#Cc1c2oc3cc4cc(N(c5ccccc5)c5cc(-c6ccccc6)ccc5-c5ccccc5)ccc4cc3c2c(C#N)c2c1oc1cc3cc(N(c4ccccc4)c4cc(-c5ccccc5)ccc4-c4ccccc4)ccc3cc12 ; N#Cc1c2oc3cc4cc(N(c5ccccc5)c5cccc(-c6ccccc6)c5)ccc4cc3c2c(C#N)c2c1oc1cc3cc(N(c4ccccc4)c4cccc(-c5ccccc5)c4)ccc3cc12 ; Cc1ccc(-c2ccccc2)cc1N(c1ccc2cc3c(cc2c1)oc1c(C#N)c2oc4cc5cc(N(c6cc(-c7ccccc7)ccc6C)c6c(C)cccc6C)ccc5cc4c2c(C#N)c13)c1c(C)cccc1C]

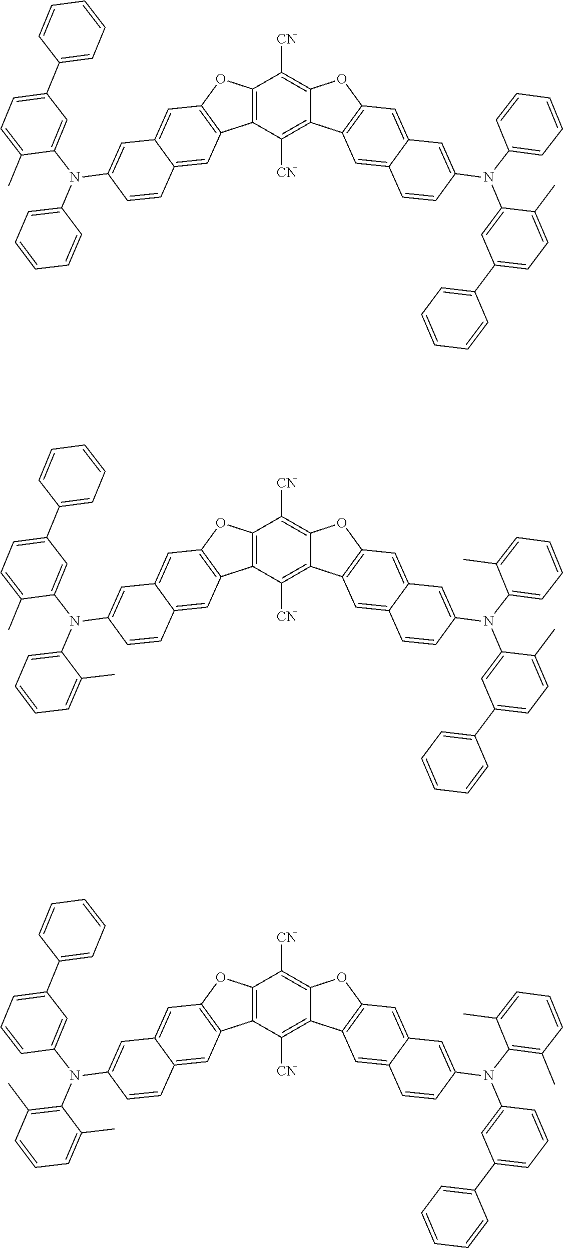 [SMILES: Cc1ccc(-c2ccccc2)cc1N(c1ccccc1)c1ccc2cc3c(cc2c1)oc1c(C#N)c2oc4cc5cc(N(c6ccccc6)c6cc(-c7ccccc7)ccc6C)ccc5cc4c2c(C#N)c13 ; Cc1ccccc1N(c1ccc2cc3c(cc2c1)oc1c(C#N)c2oc4cc5cc(N(c6ccccc6C)c6cc(-c7ccccc7)ccc6C)ccc5cc4c2c(C#N)c13)c1cc(-c2ccccc2)ccc1C ; Cc1cccc(C)c1N(c1cccc(-c2ccccc2)c1)c1ccc2cc3c(cc2c1)oc1c(C#N)c2oc4cc5cc(N(c6cccc(-c7ccccc7)c6)c6c(C)cccc6C)ccc5cc4c2c(C#N)c13]

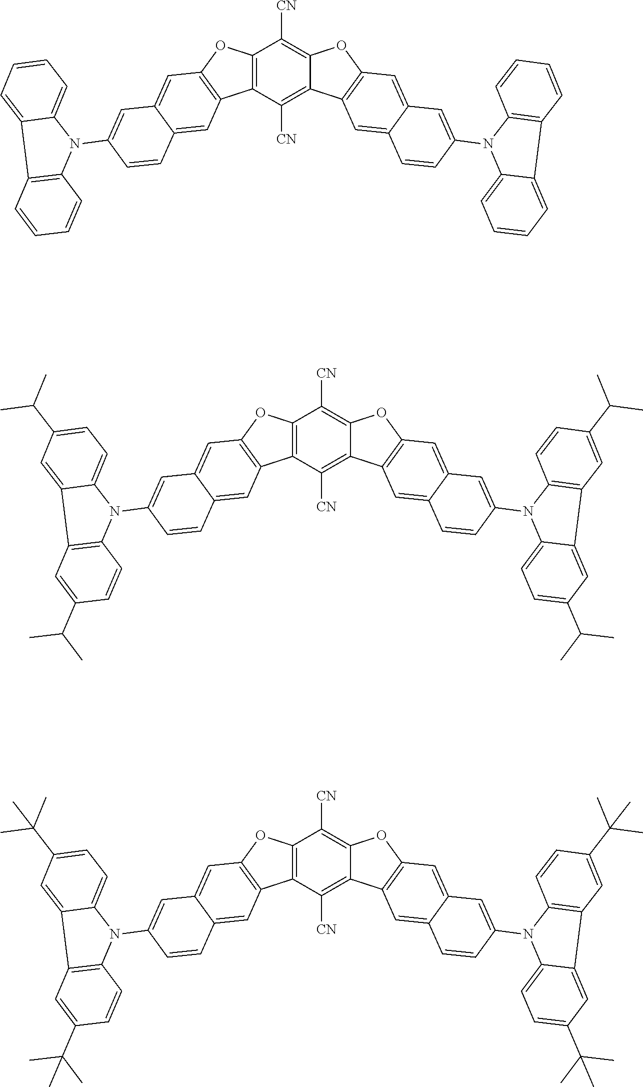

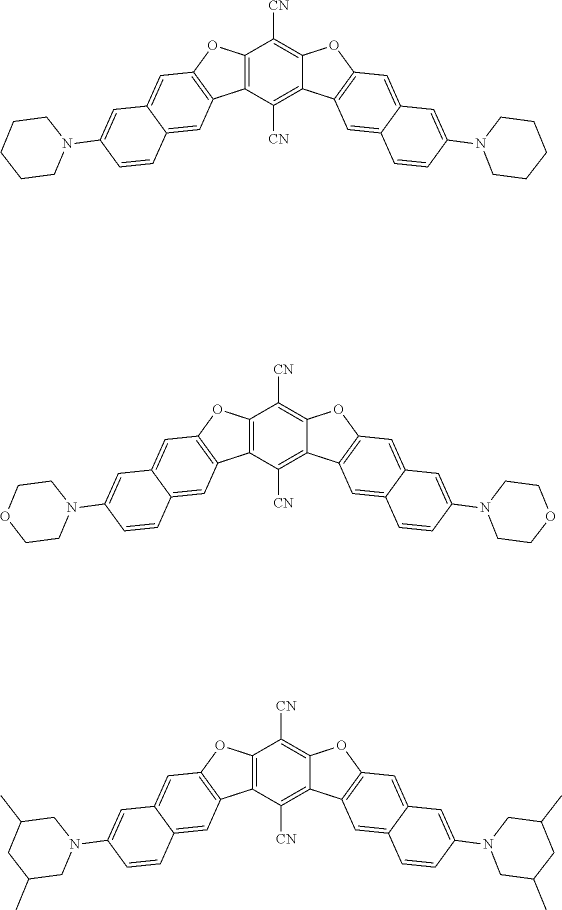

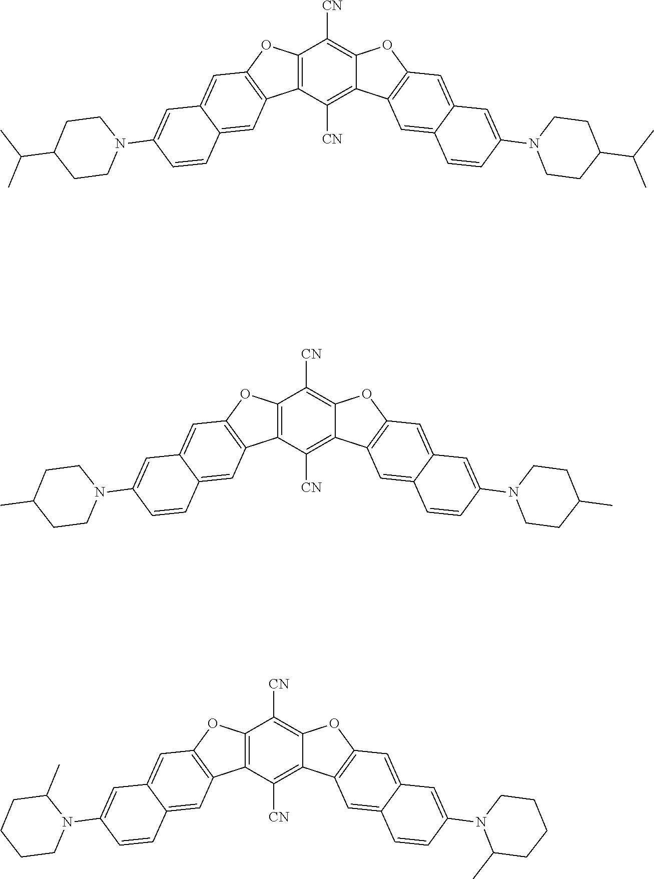

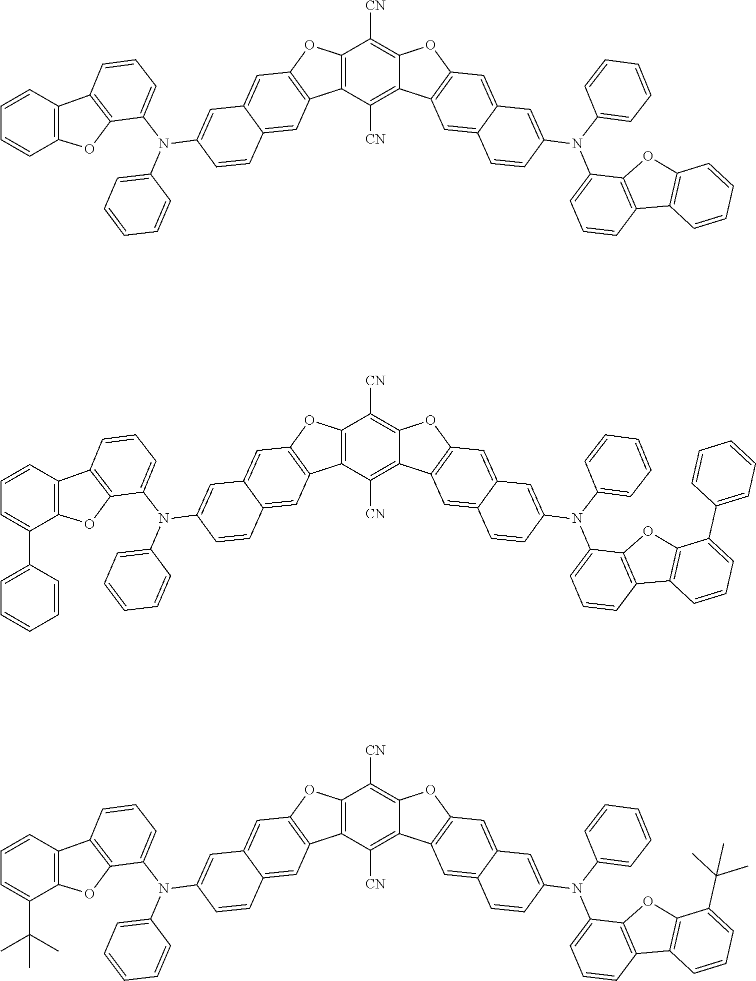 [SMILES: N#Cc1c2oc3cc4cc(N(c5ccccc5)c5cccc6c5oc5ccccc56)ccc4cc3c2c(C#N)c2c1oc1cc3cc(N(c4ccccc4)c4cccc5c4oc4ccccc45)ccc3cc12 ; N#Cc1c2oc3cc4cc(N(c5ccccc5)c5cccc6c5oc5c(-c7ccccc7)cccc56)ccc4cc3c2c(C#N)c2c1oc1cc3cc(N(c4ccccc4)c4cccc5c4oc4c(-c6ccccc6)cccc45)ccc3cc12 ; CC(C)(C)c1cccc2c1oc1c(N(c3ccccc3)c3ccc4cc5c(cc4c3)oc3c(C#N)c4oc6cc7cc(N(c8ccccc8)c8cccc9c8oc8c(C(C)(C)C)cccc89)ccc7cc6c4c(C#N)c35)cccc12]

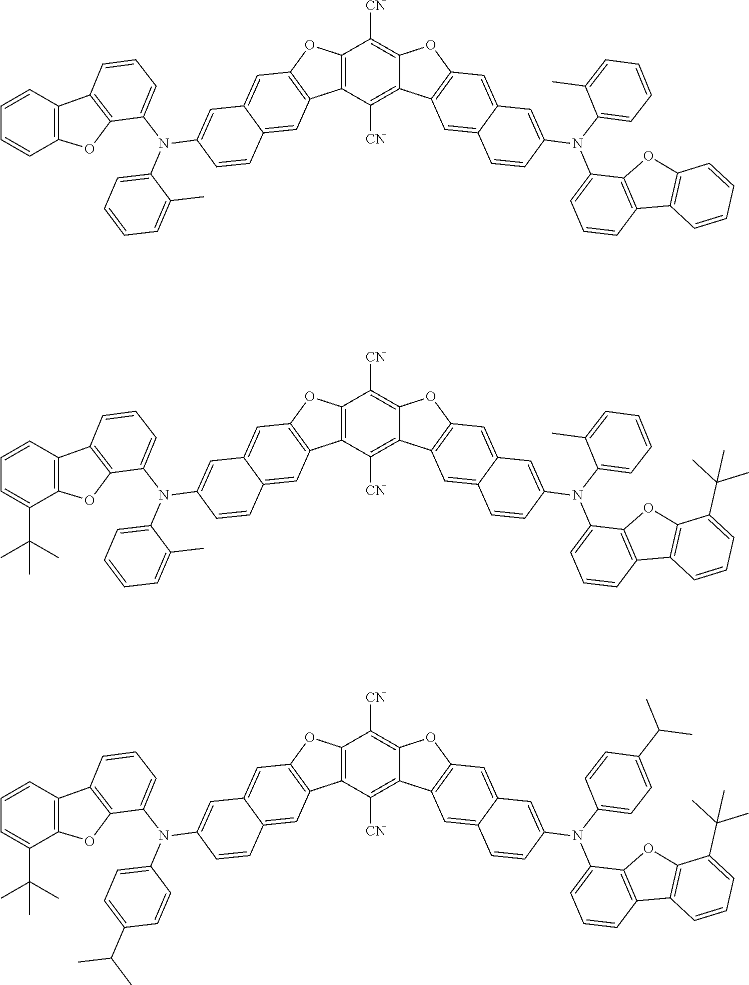

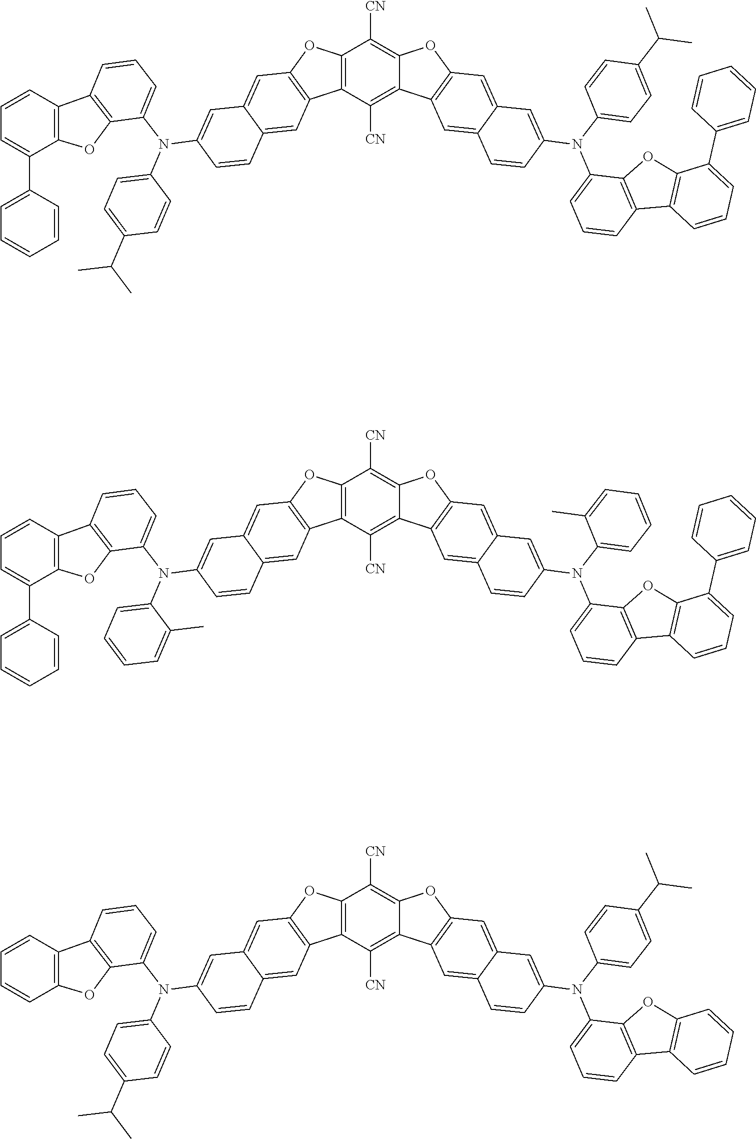 [SMILES: CC(C)c1ccc(N(c2ccc3cc4c(cc3c2)oc2c(C#N)c3oc5cc6cc(N(c7ccc(C(C)C)cc7)c7cccc8c7oc7c(-c9ccccc9)cccc78)ccc6cc5c3c(C#N)c24)c2cccc3c2oc2c(-c4ccccc4)cccc23)cc1 ; Cc1ccccc1N(c1ccc2cc3c(cc2c1)oc1c(C#N)c2oc4cc5cc(N(c6ccccc6C)c6cccc7c6oc6c(-c8ccccc8)cccc67)ccc5cc4c2c(C#N)c13)c1cccc2c1oc1c(-c3ccccc3)cccc12 ; CC(C)c1ccc(N(c2ccc3cc4c(cc3c2)oc2c(C#N)c3oc5cc6cc(N(c7ccc(C(C)C)cc7)c7cccc8c7oc7ccccc78)ccc6cc5c3c(C#N)c24)c2cccc3c2oc2ccccc23)cc1]

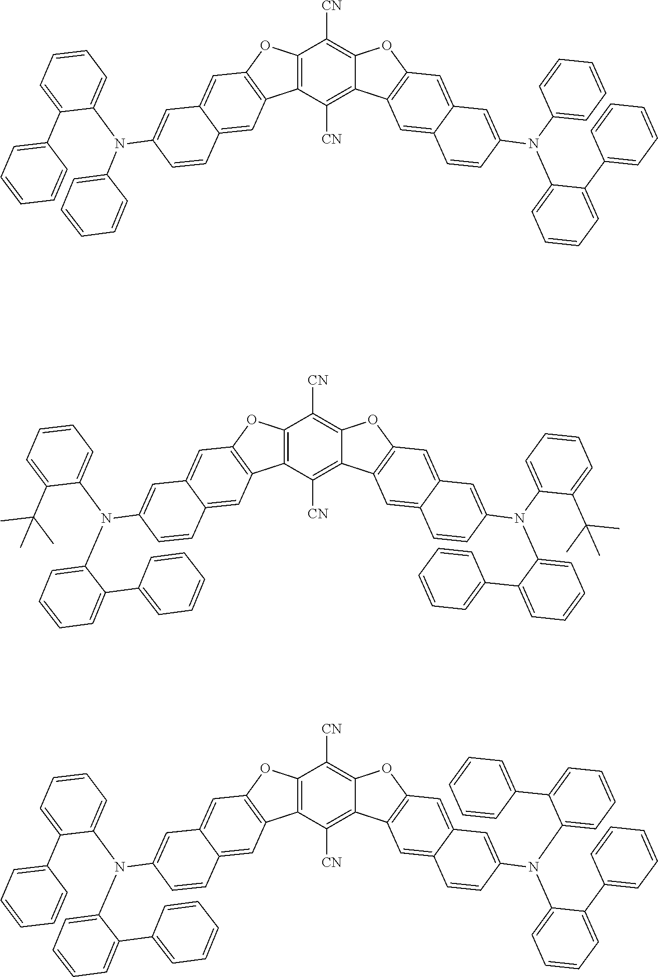 [SMILES: N#Cc1c2oc3cc4cc(N(c5ccccc5)c5ccccc5-c5ccccc5)ccc4cc3c2c(C#N)c2c1oc1cc3cc(N(c4ccccc4)c4ccccc4-c4ccccc4)ccc3cc12 ; CC(C)(C)c1ccccc1N(c1ccc2cc3c(cc2c1)oc1c(C#N)c2oc4cc5cc(N(c6ccccc6-c6ccccc6)c6ccccc6C(C)(C)C)ccc5cc4c2c(C#N)c13)c1ccccc1-c1ccccc1 ; N#Cc1c2oc3cc4cc(N(c5ccccc5-c5ccccc5)c5ccccc5-c5ccccc5)ccc4cc3c2c(C#N)c2c1oc1cc3cc(N(c4ccccc4-c4ccccc4)c4ccccc4-c4ccccc4)ccc3cc12]

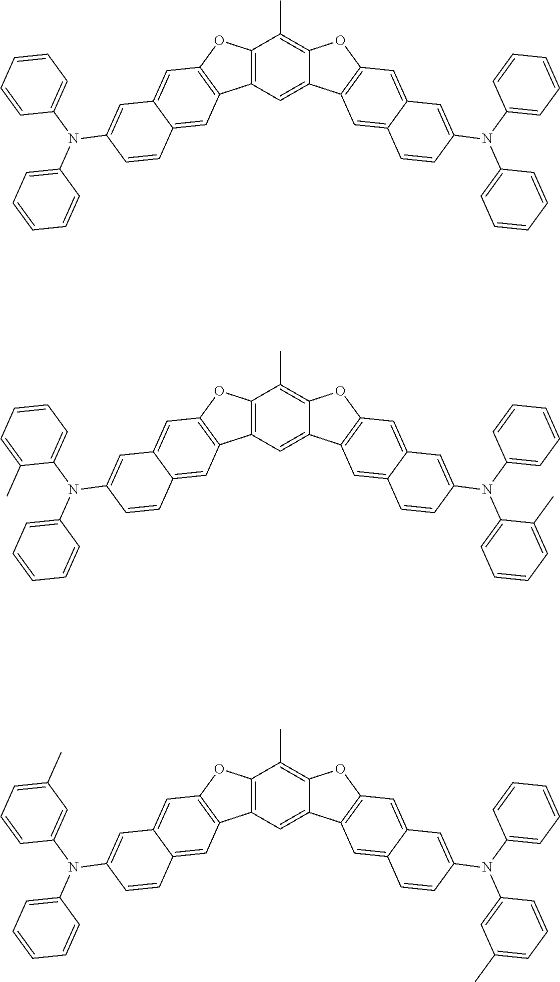 [SMILES: Cc1c2oc3cc4cc(N(c5ccccc5)c5ccccc5)ccc4cc3c2cc2c1oc1cc3cc(N(c4ccccc4)c4ccccc4)ccc3cc12 ; Cc1ccccc1N(c1ccccc1)c1ccc2cc3c(cc2c1)oc1c(C)c2oc4cc5cc(N(c6ccccc6)c6ccccc6C)ccc5cc4c2cc13 ; Cc1cccc(N(c2ccccc2)c2ccc3cc4c(cc3c2)oc2c(C)c3oc5cc6cc(N(c7ccccc7)c7cccc(C)c7)ccc6cc5c3cc24)c1]

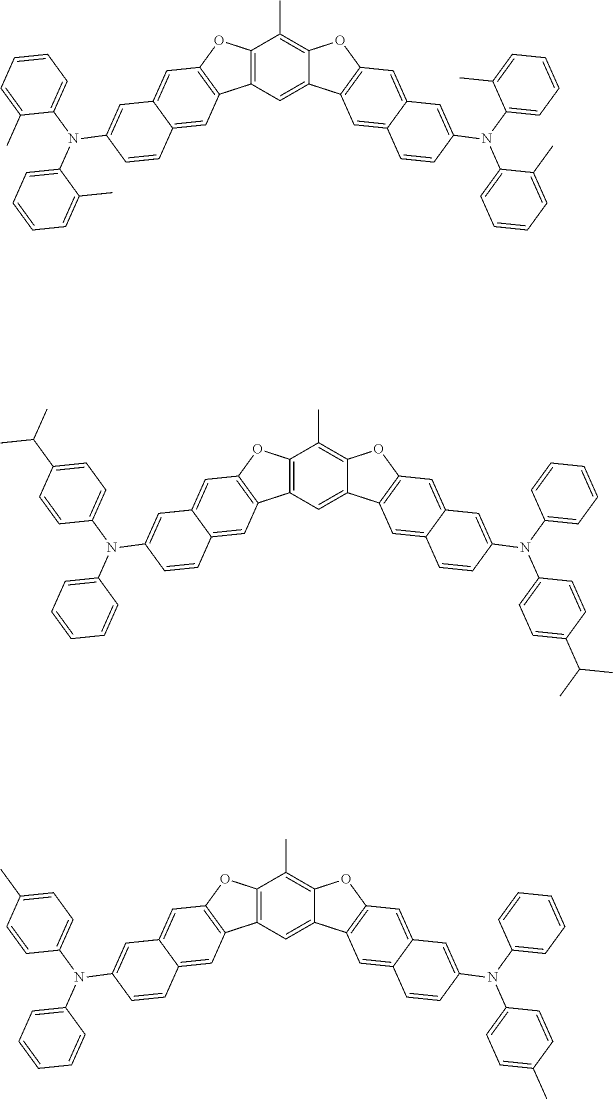

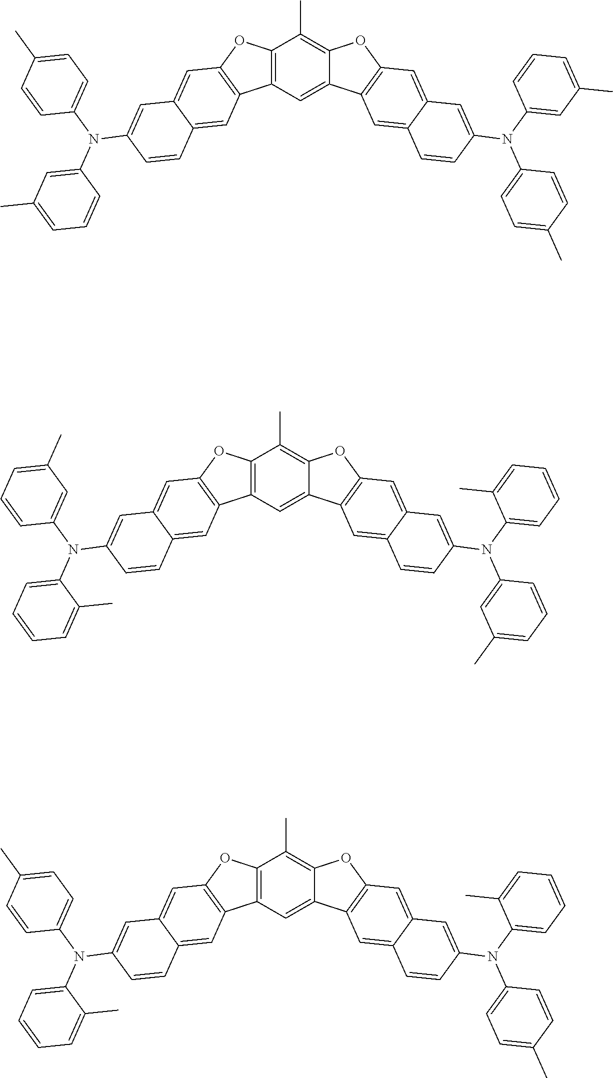

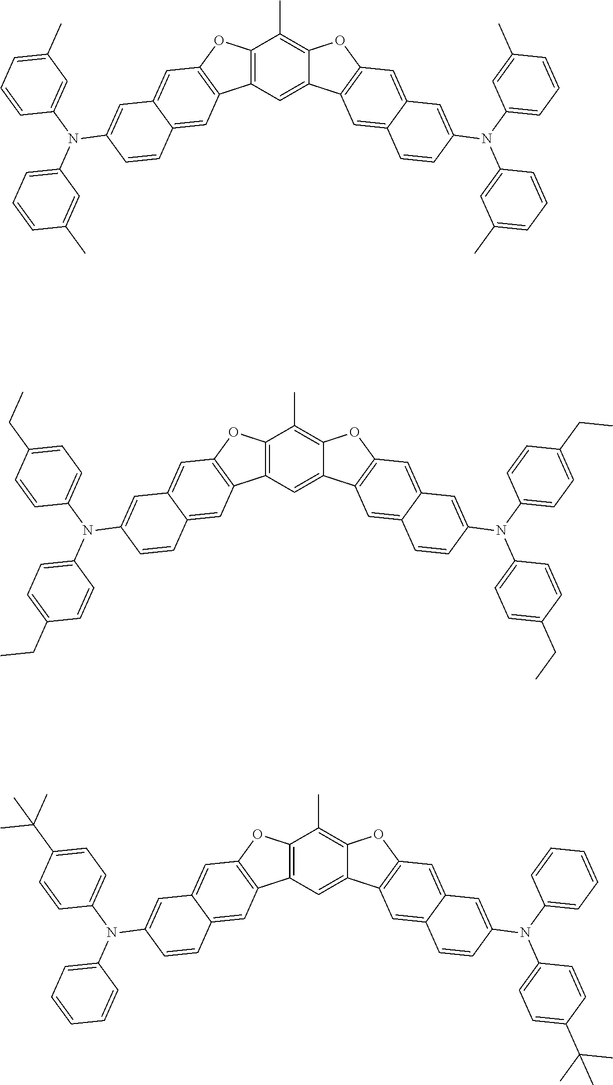

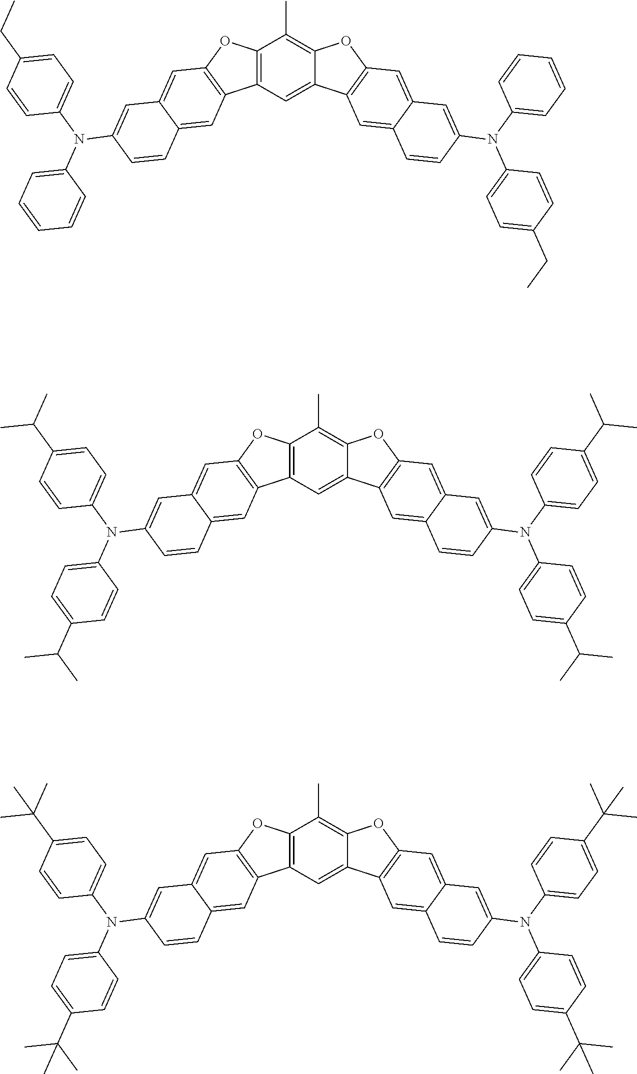

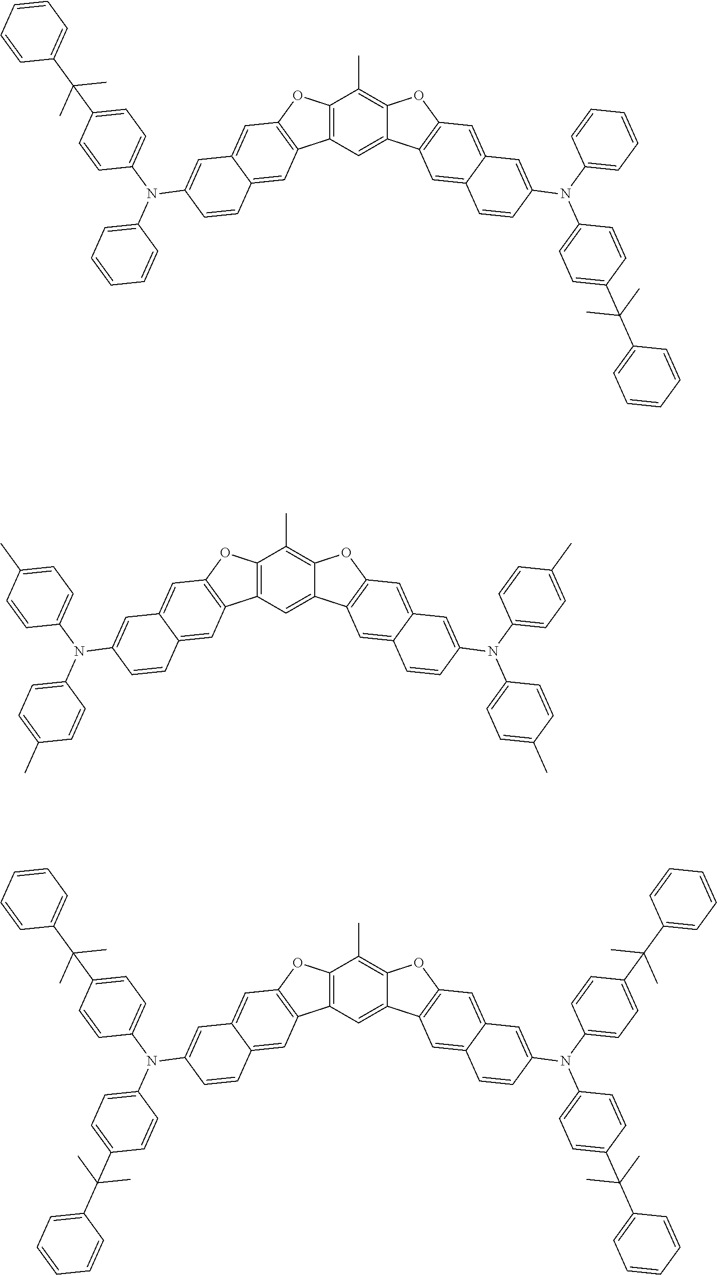

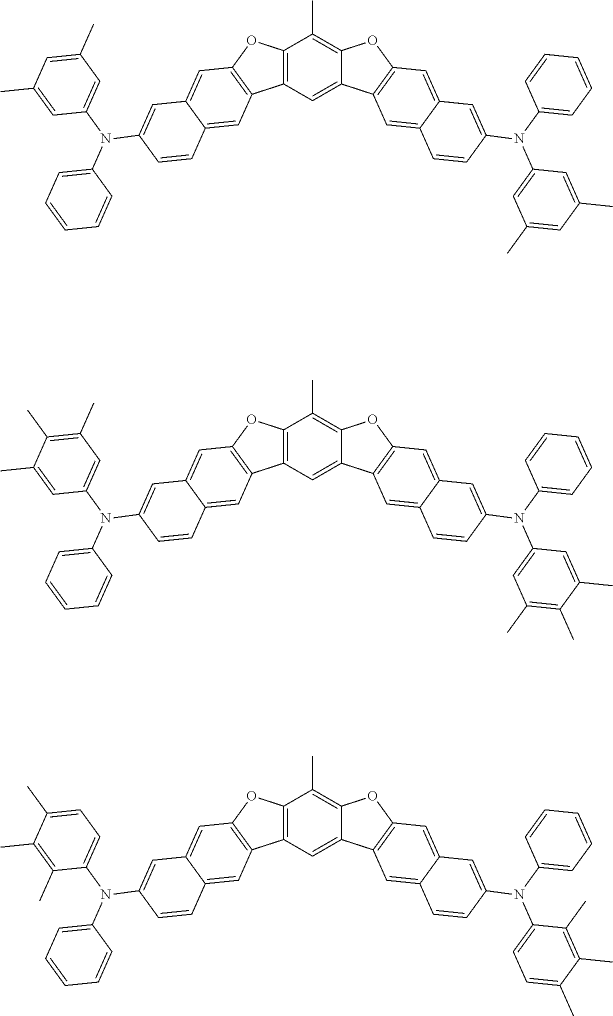

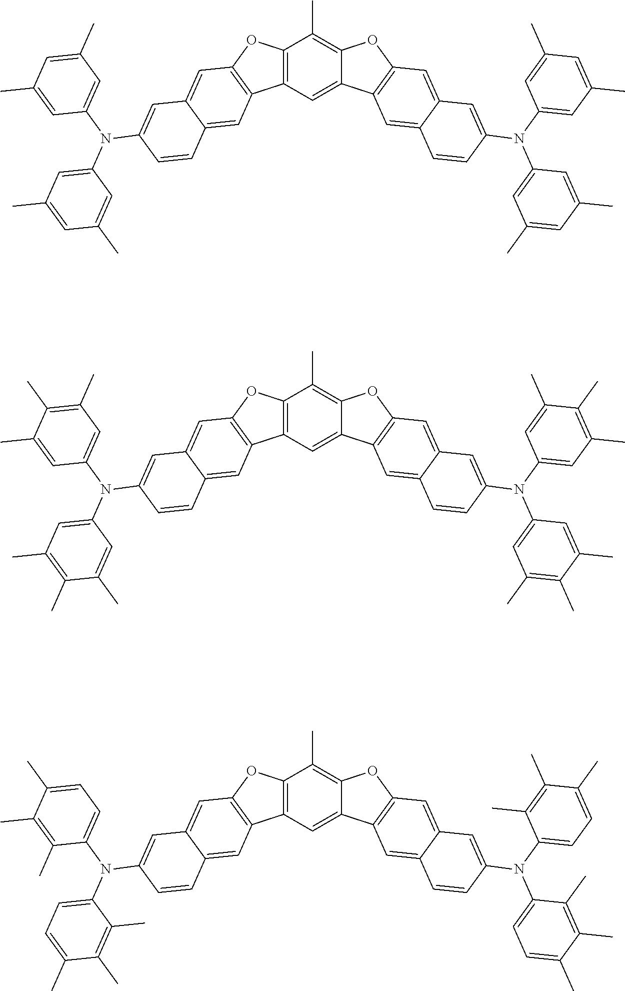

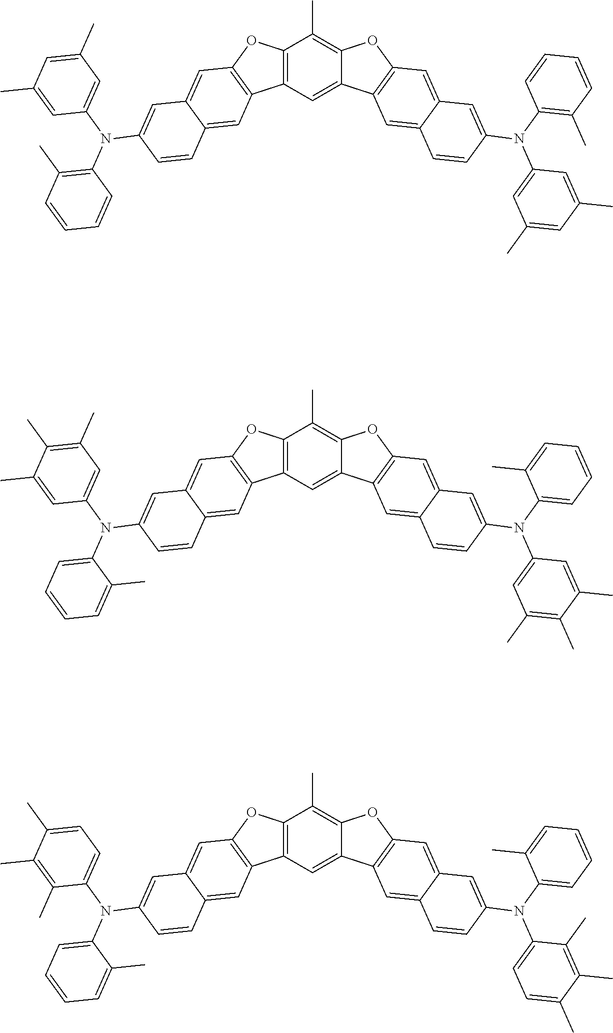

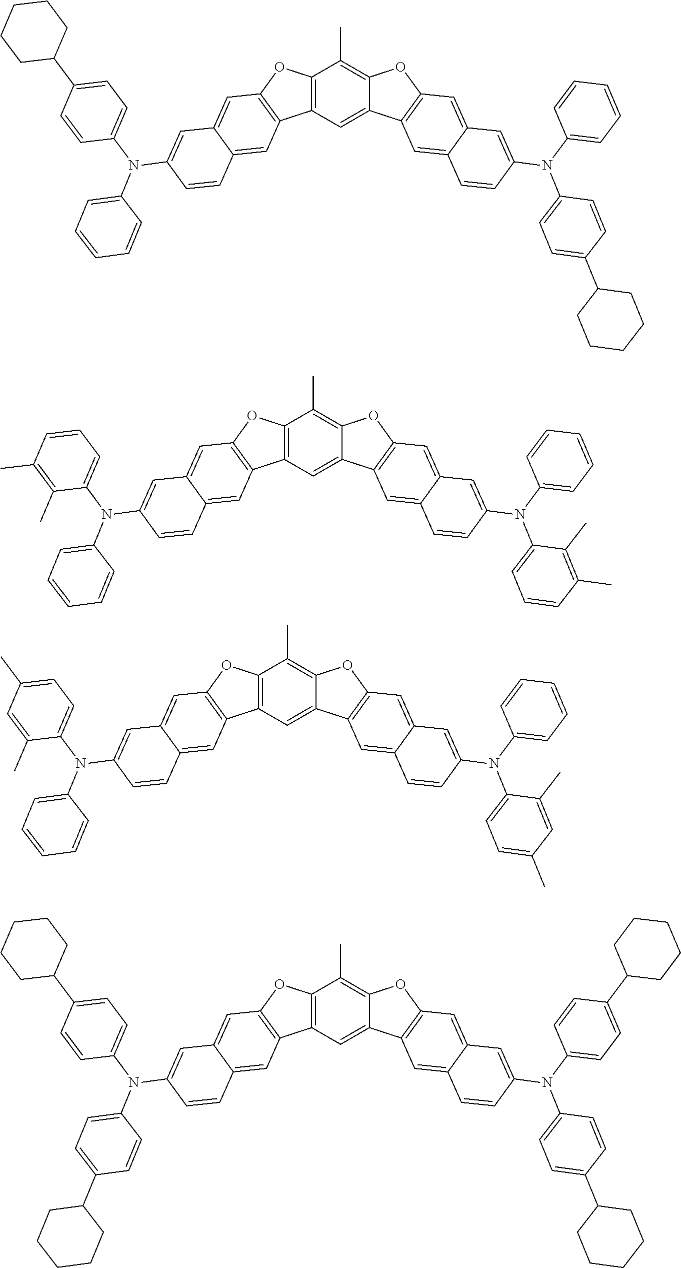

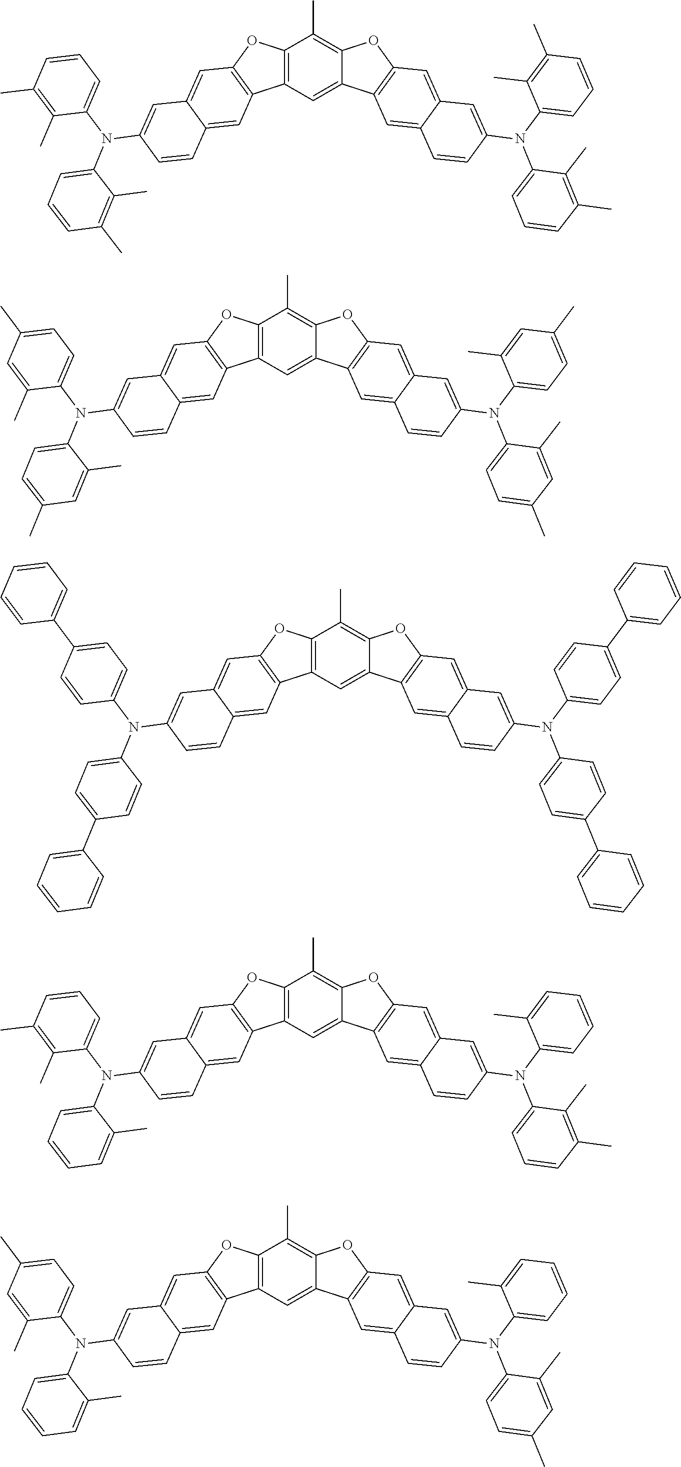

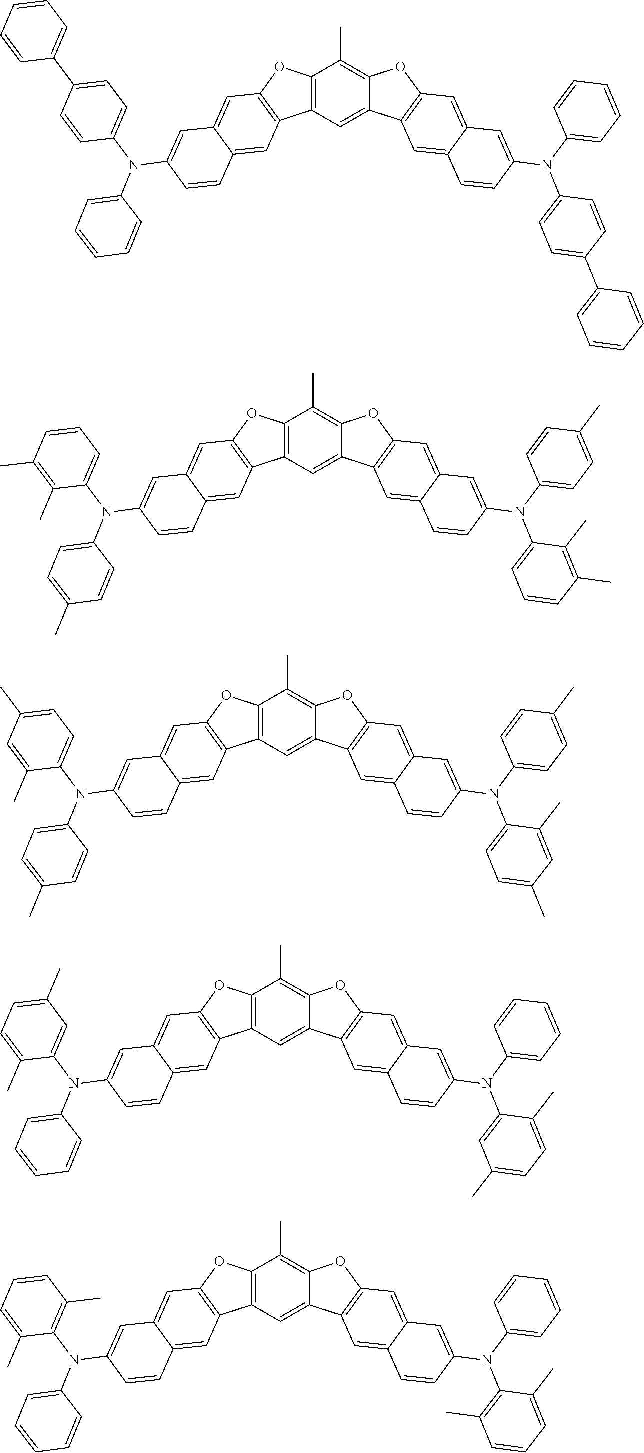

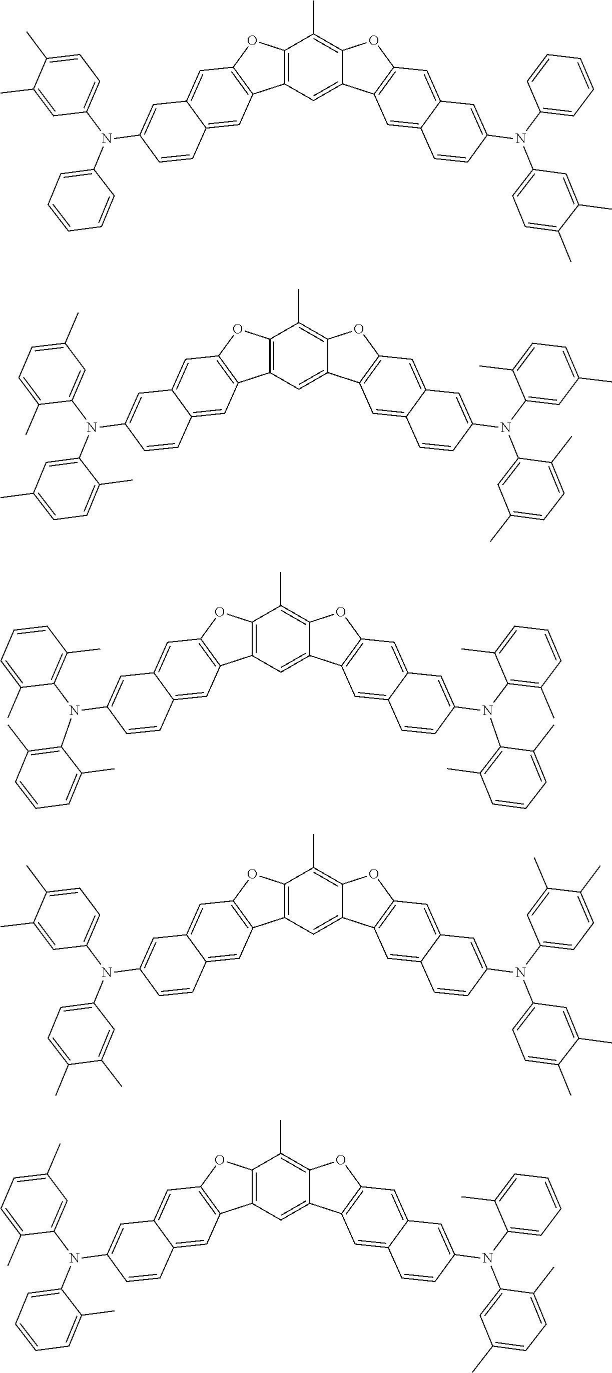

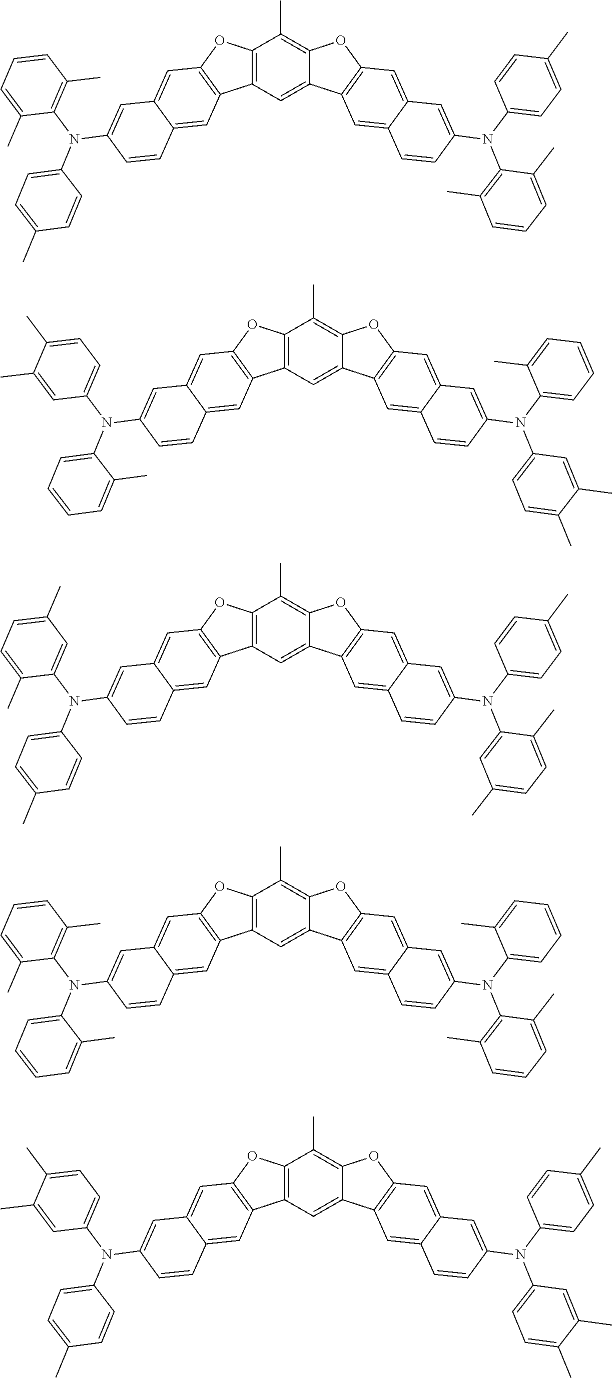

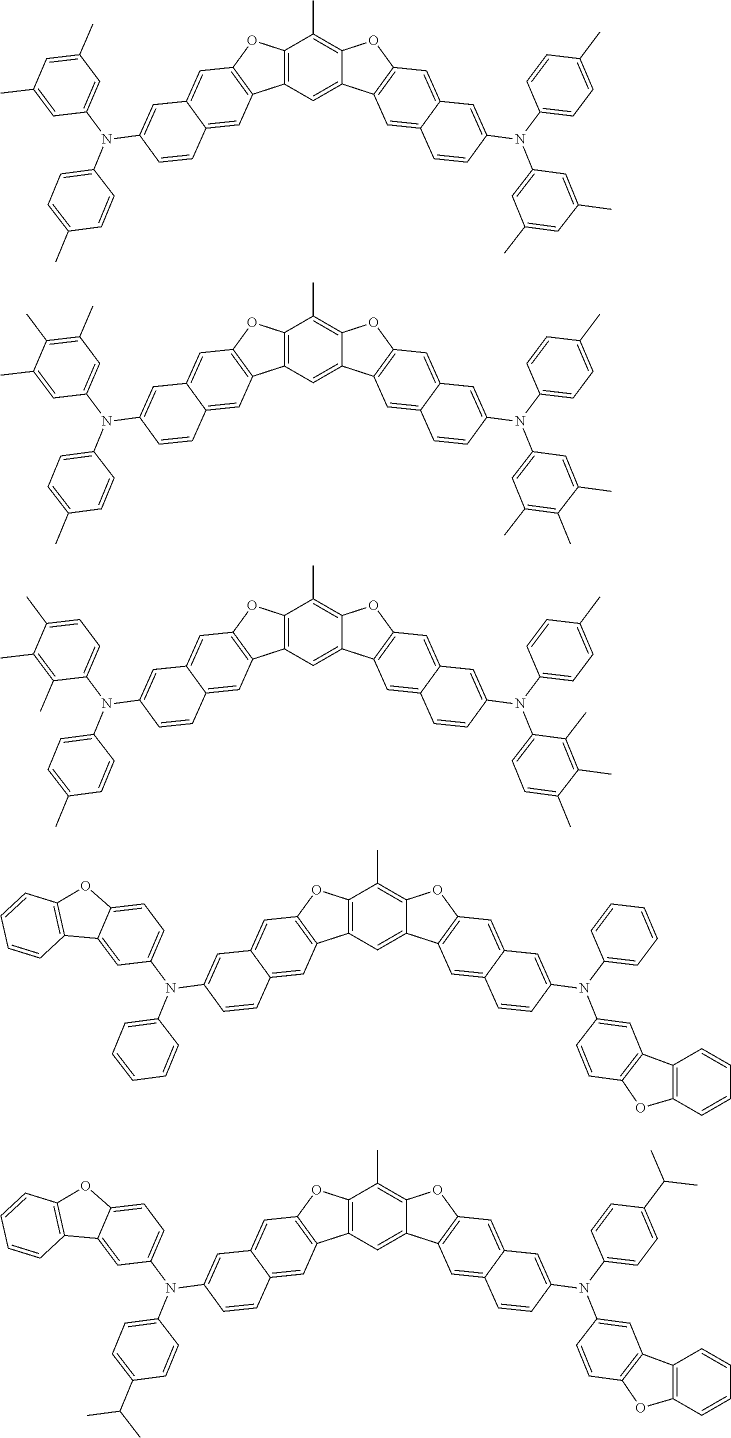

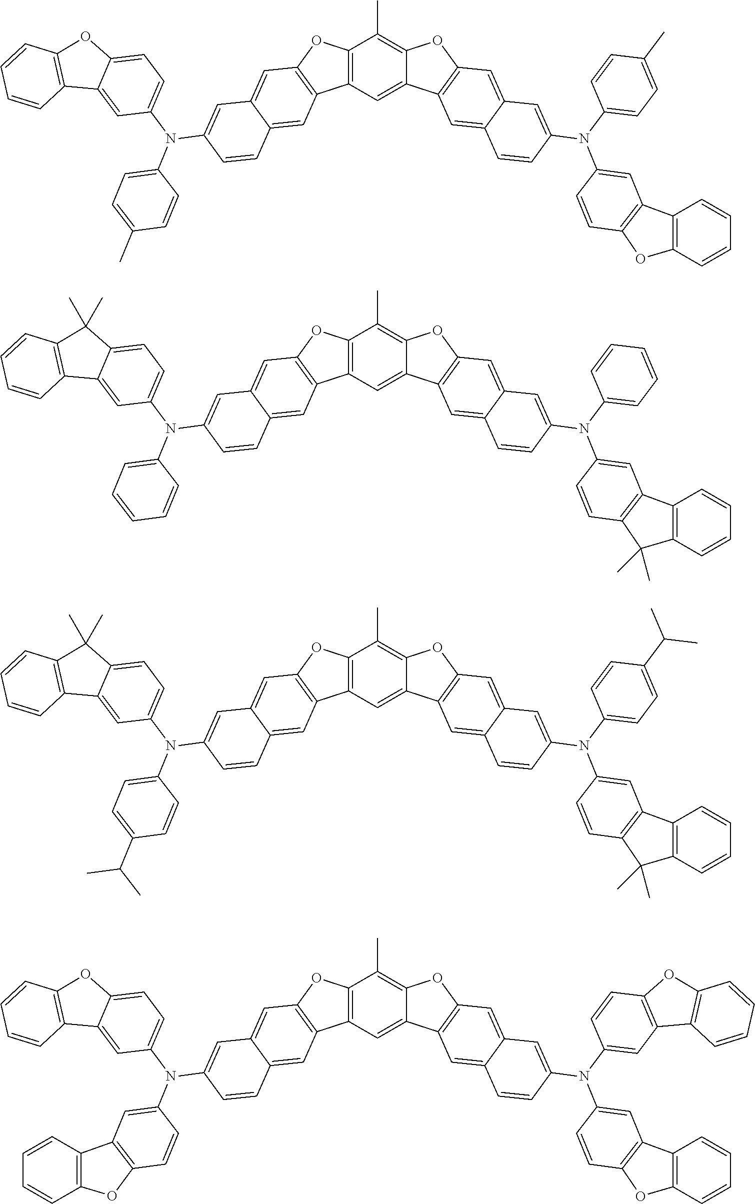

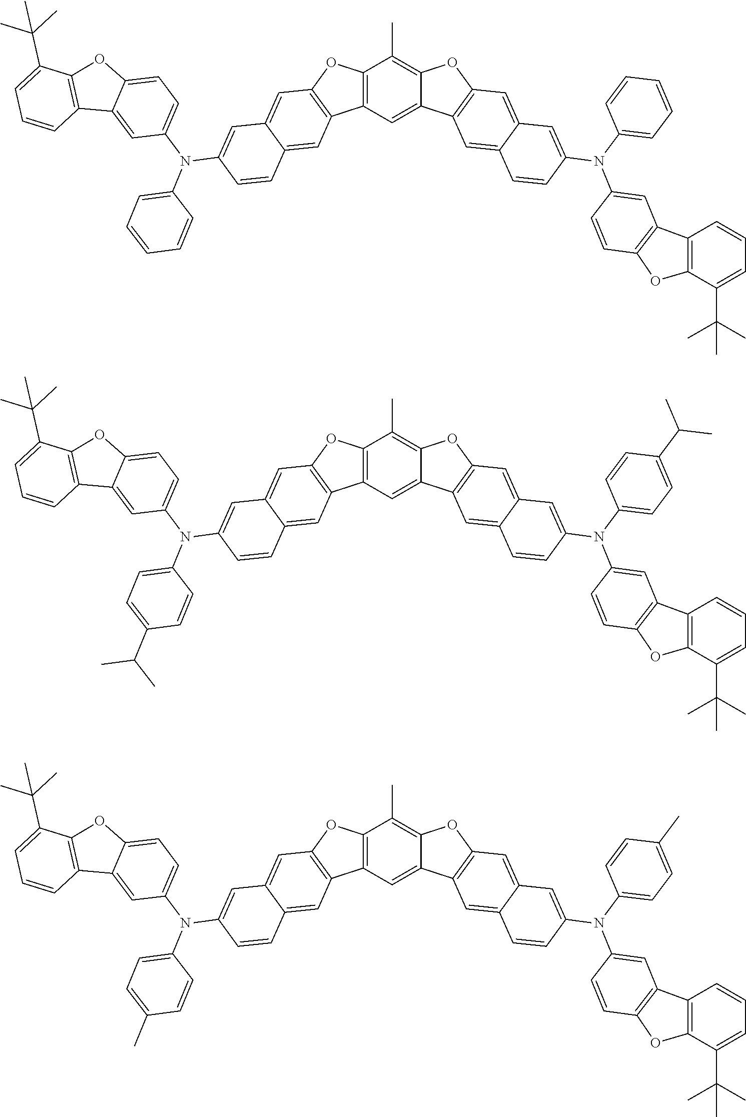

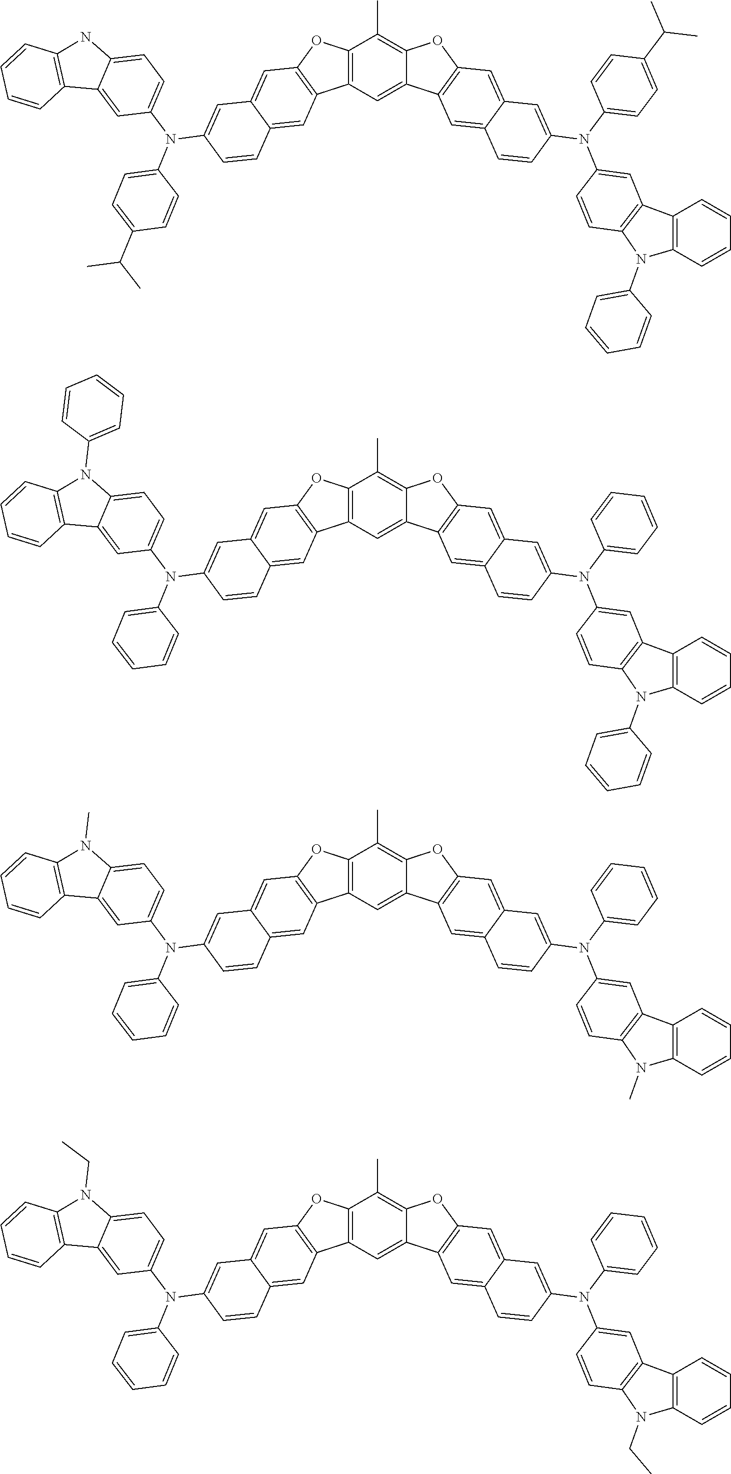

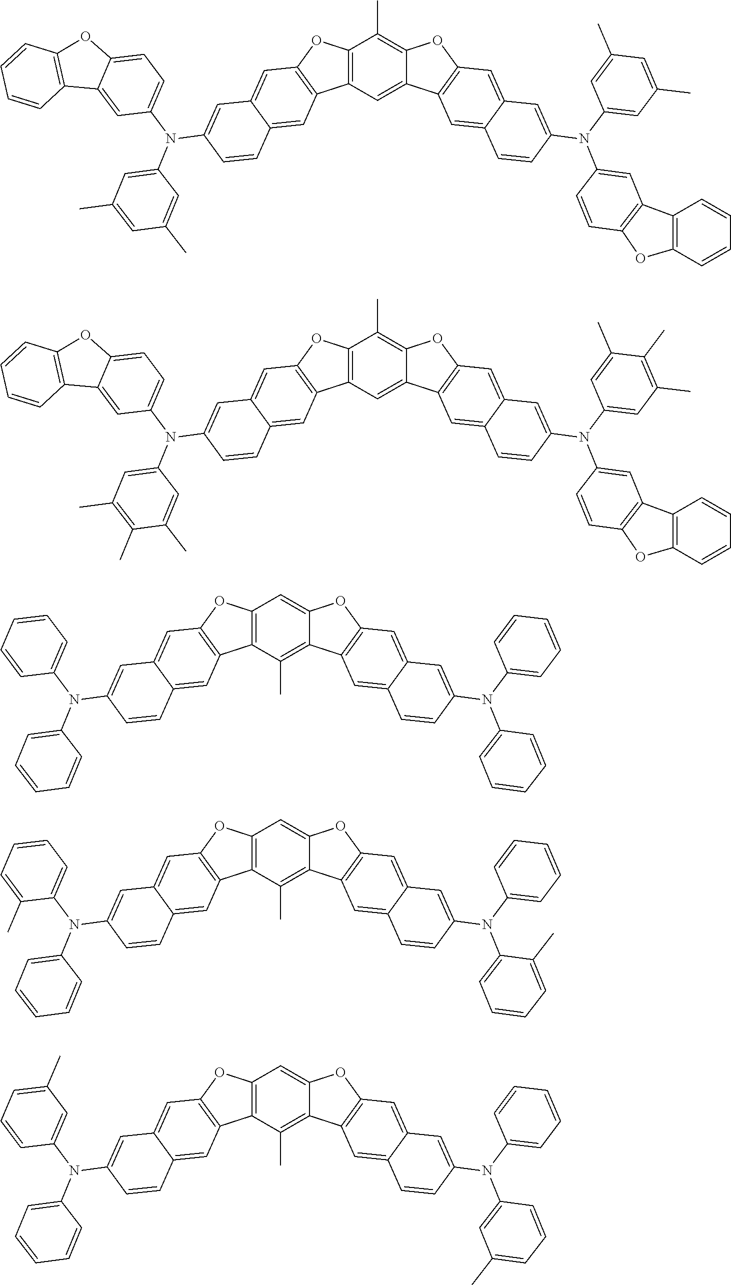

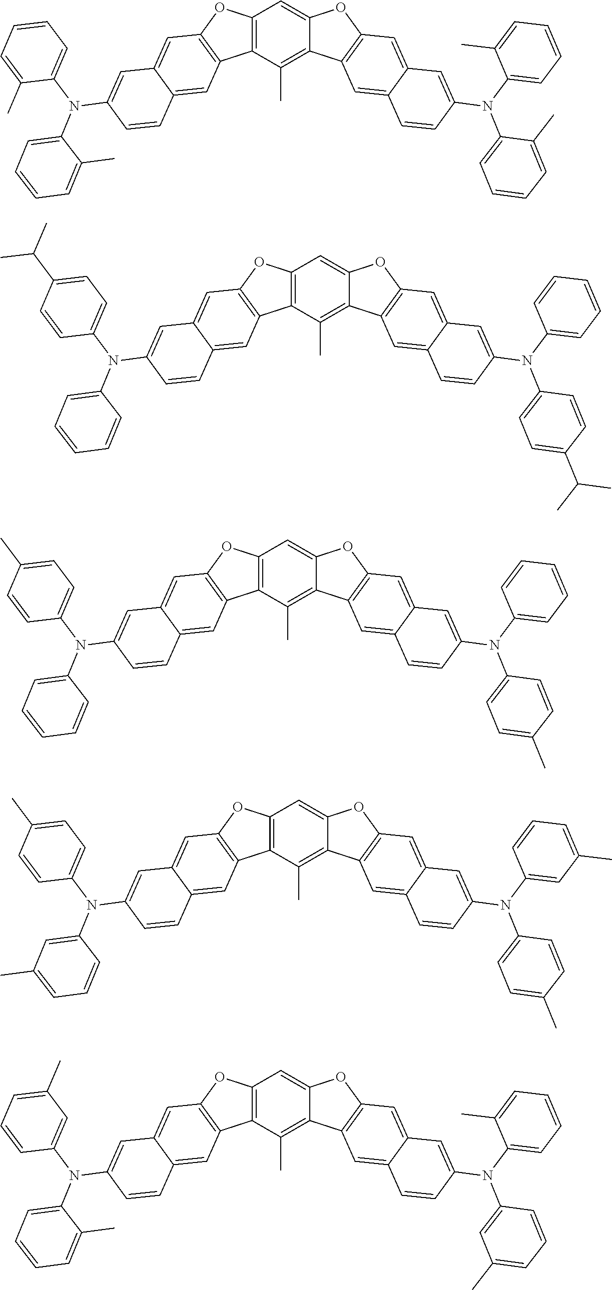

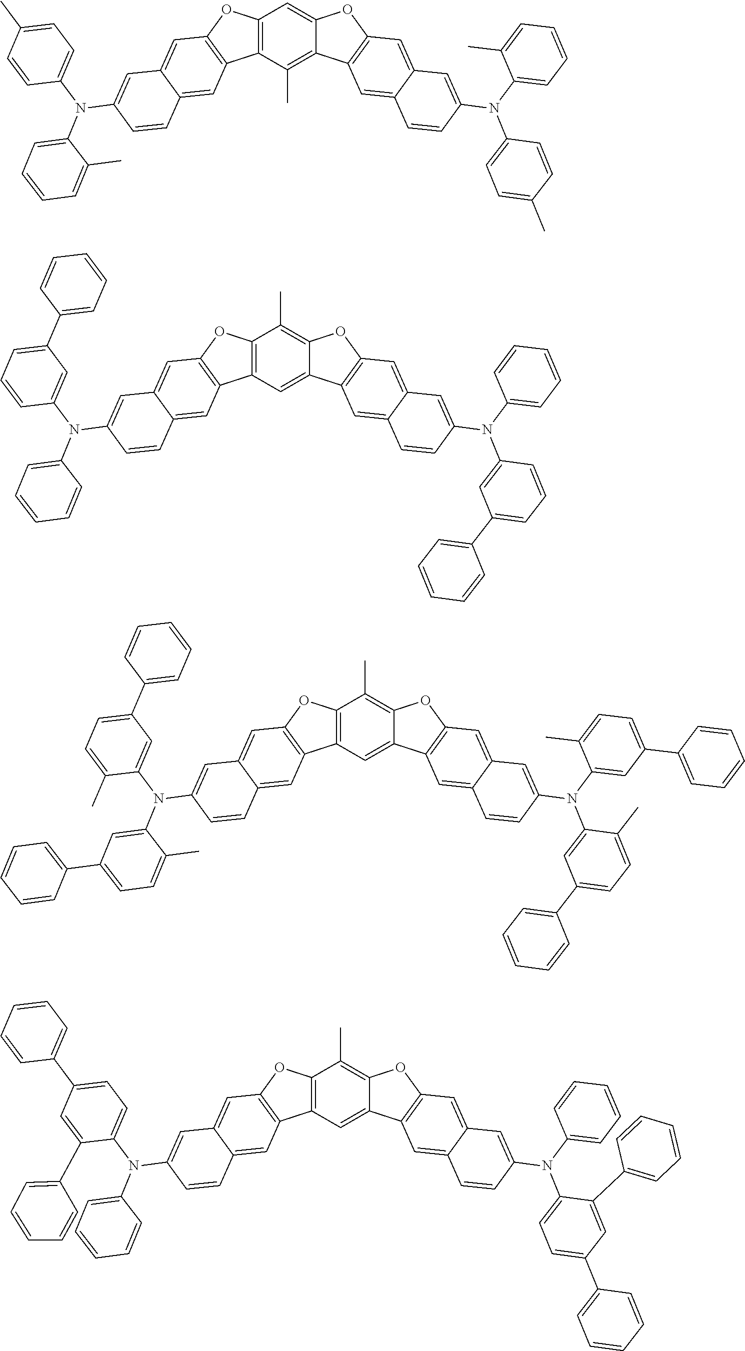 [SMILES: Cc1ccc(N(c2ccc3cc4c(cc3c2)oc2cc3oc5cc6cc(N(c7ccc(C)cc7)c7ccccc7C)ccc6cc5c3c(C)c24)c2ccccc2C)cc1 ; Cc1c2oc3cc4cc(N(c5ccccc5)c5cccc(-c6ccccc6)c5)ccc4cc3c2cc2c1oc1cc3cc(N(c4ccccc4)c4cccc(-c5ccccc5)c4)ccc3cc12 ; Cc1ccc(-c2ccccc2)cc1N(c1ccc2cc3c(cc2c1)oc1c(C)c2oc4cc5cc(N(c6cc(-c7ccccc7)ccc6C)c6cc(-c7ccccc7)ccc6C)ccc5cc4c2cc13)c1cc(-c2ccccc2)ccc1C ; Cc1c2oc3cc4cc(N(c5ccccc5)c5ccc(-c6ccccc6)cc5-c5ccccc5)ccc4cc3c2cc2c1oc1cc3cc(N(c4ccccc4)c4ccc(-c5ccccc5)cc4-c4ccccc4)ccc3cc12]

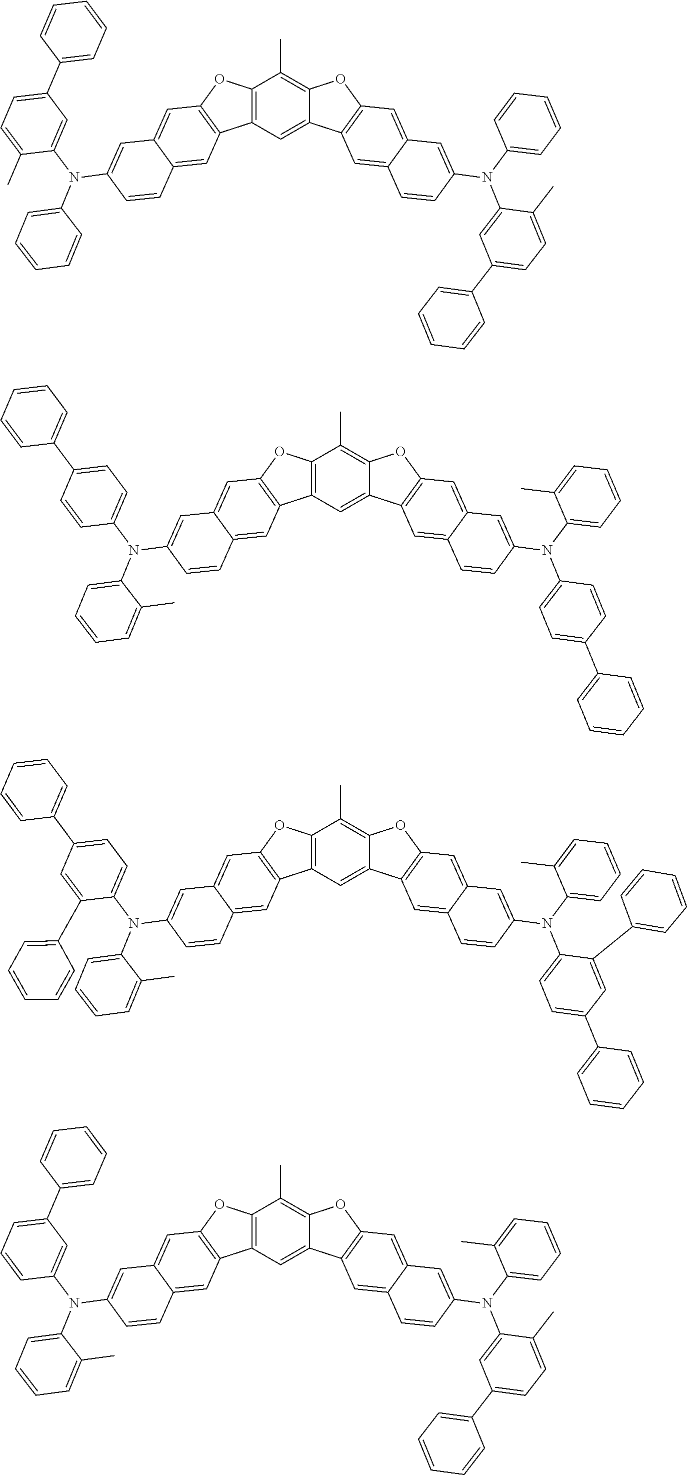 [SMILES: Cc1ccc(-c2ccccc2)cc1N(c1ccccc1)c1ccc2cc3c(cc2c1)oc1c(C)c2oc4cc5cc(N(c6ccccc6)c6cc(-c7ccccc7)ccc6C)ccc5cc4c2cc13 ; Cc1ccccc1N(c1ccc(-c2ccccc2)cc1)c1ccc2cc3c(cc2c1)oc1c(C)c2oc4cc5cc(N(c6ccc(-c7ccccc7)cc6)c6ccccc6C)ccc5cc4c2cc13 ; Cc1ccccc1N(c1ccc2cc3c(cc2c1)oc1c(C)c2oc4cc5cc(N(c6ccccc6C)c6ccc(-c7ccccc7)cc6-c6ccccc6)ccc5cc4c2cc13)c1ccc(-c2ccccc2)cc1-c1ccccc1 ; Cc1ccccc1N(c1cccc(-c2ccccc2)c1)c1ccc2cc3c(cc2c1)oc1c(C)c2oc4cc5cc(N(c6ccccc6C)c6cc(-c7ccccc7)ccc6C)ccc5cc4c2cc13]

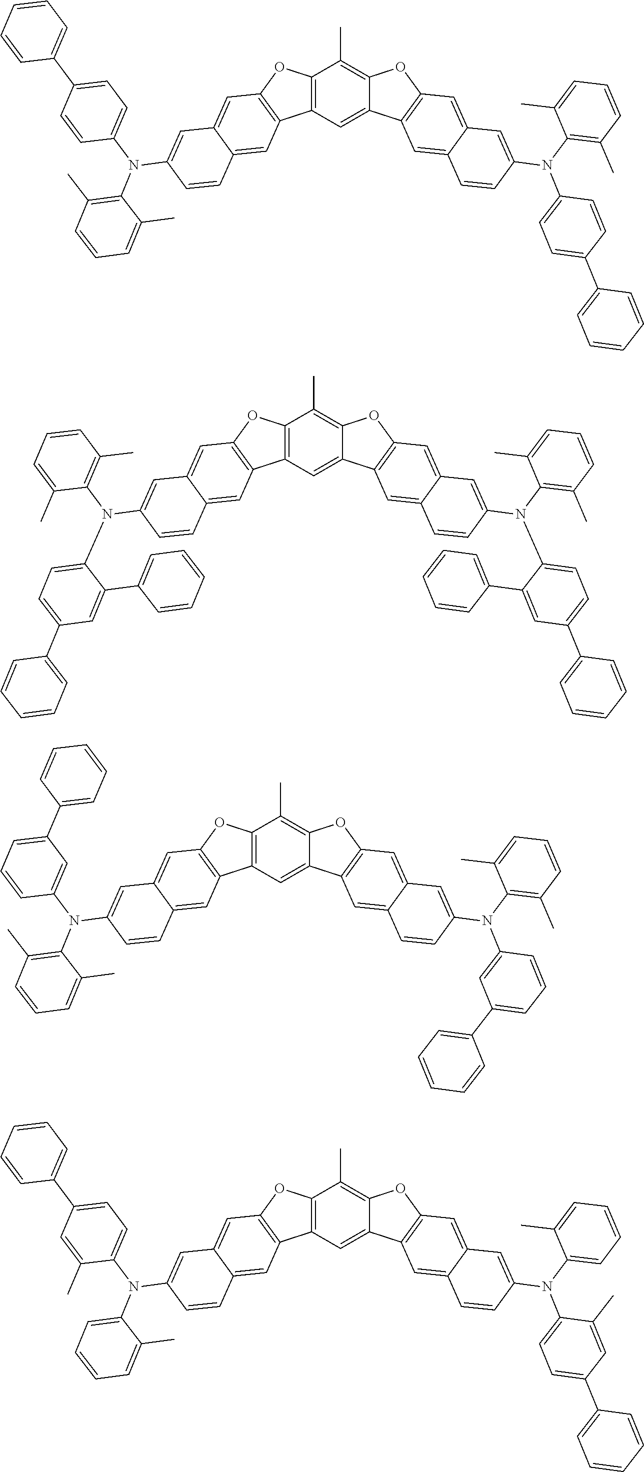

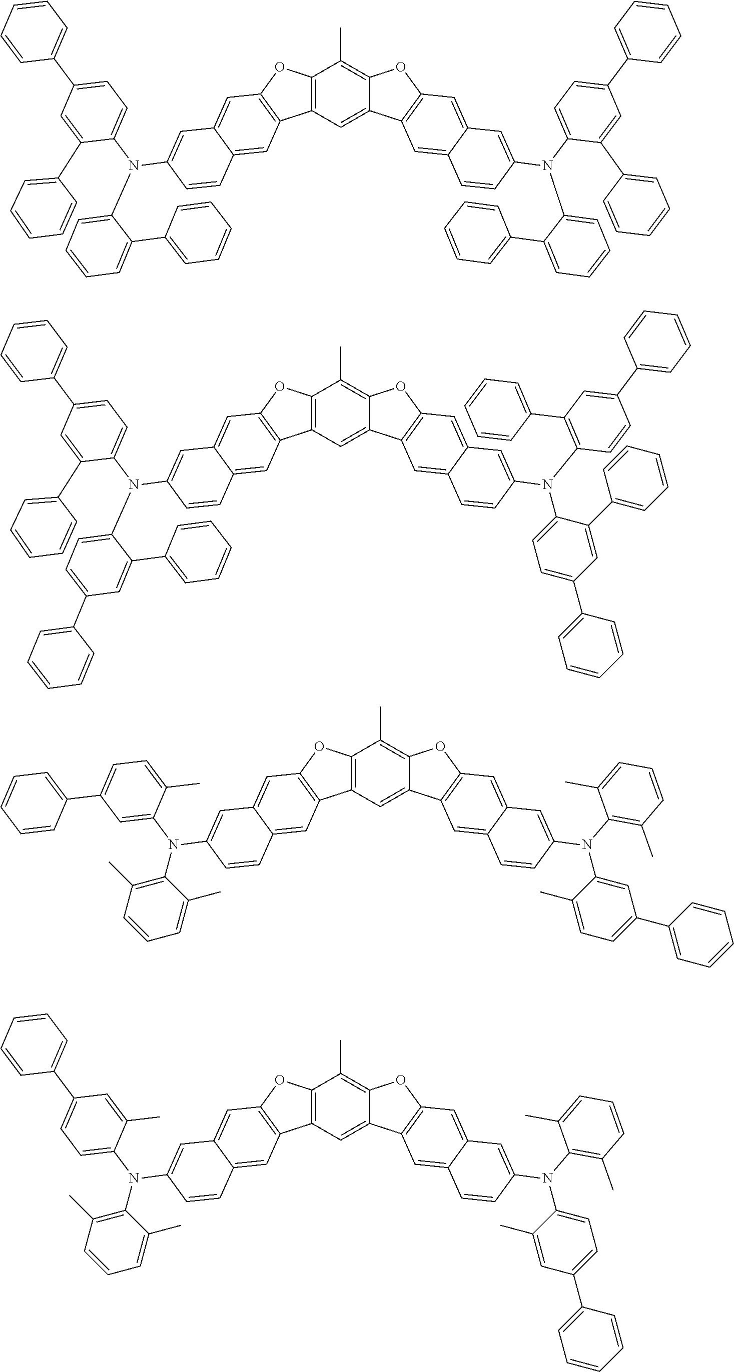

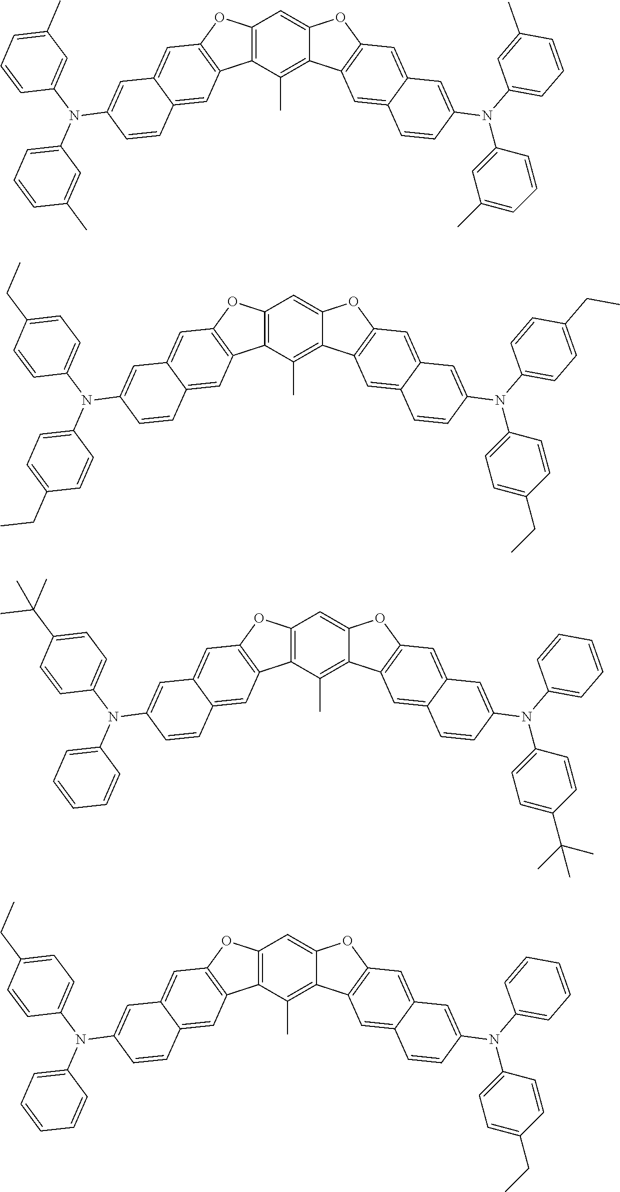

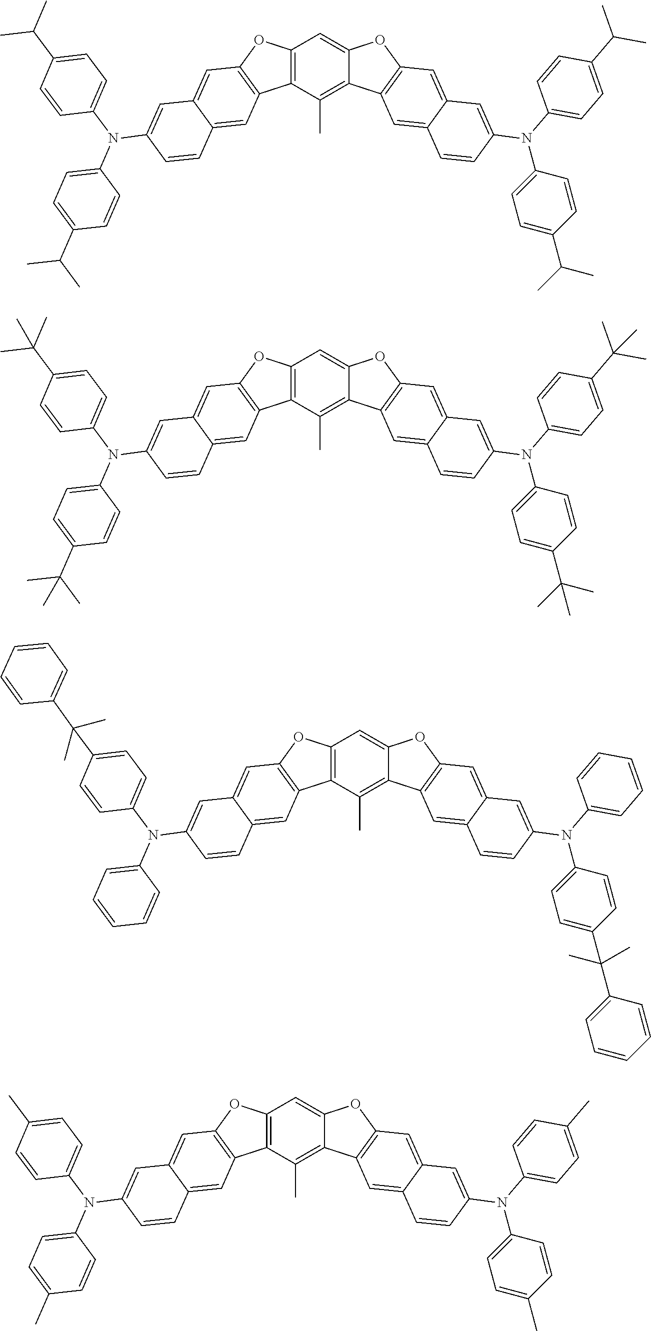

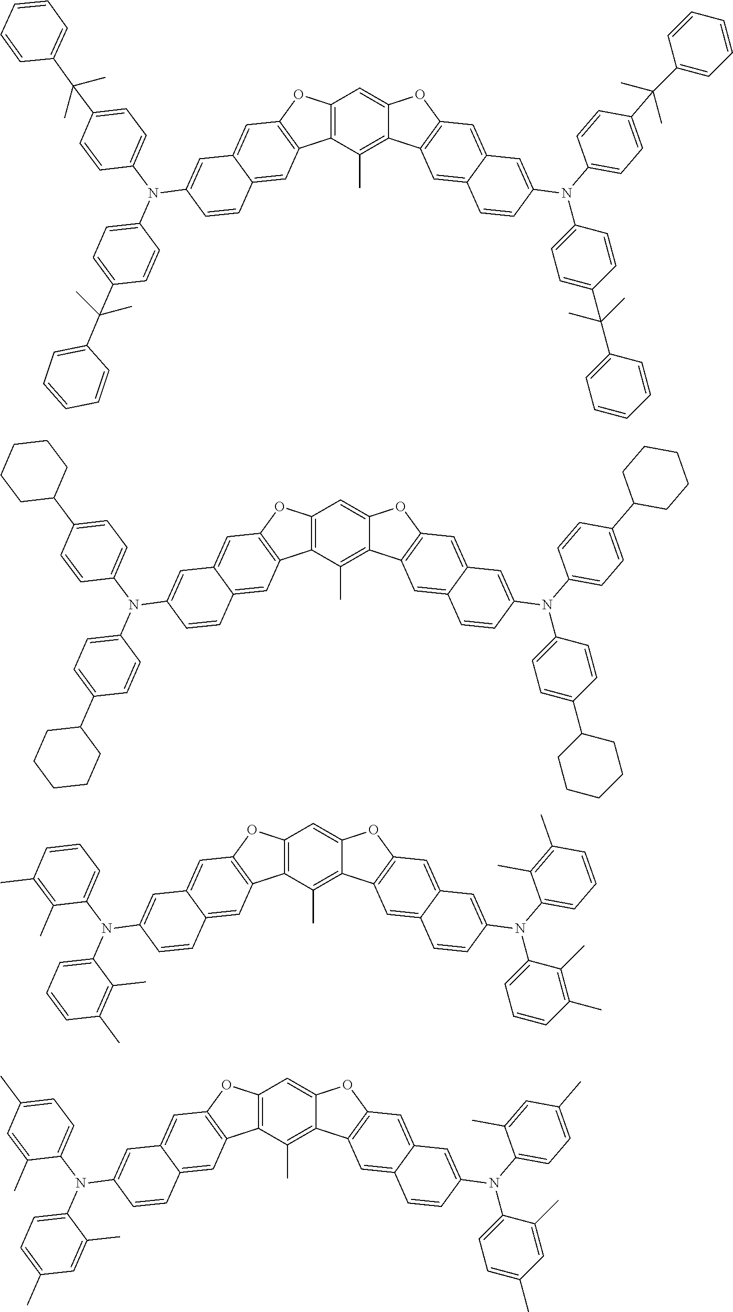

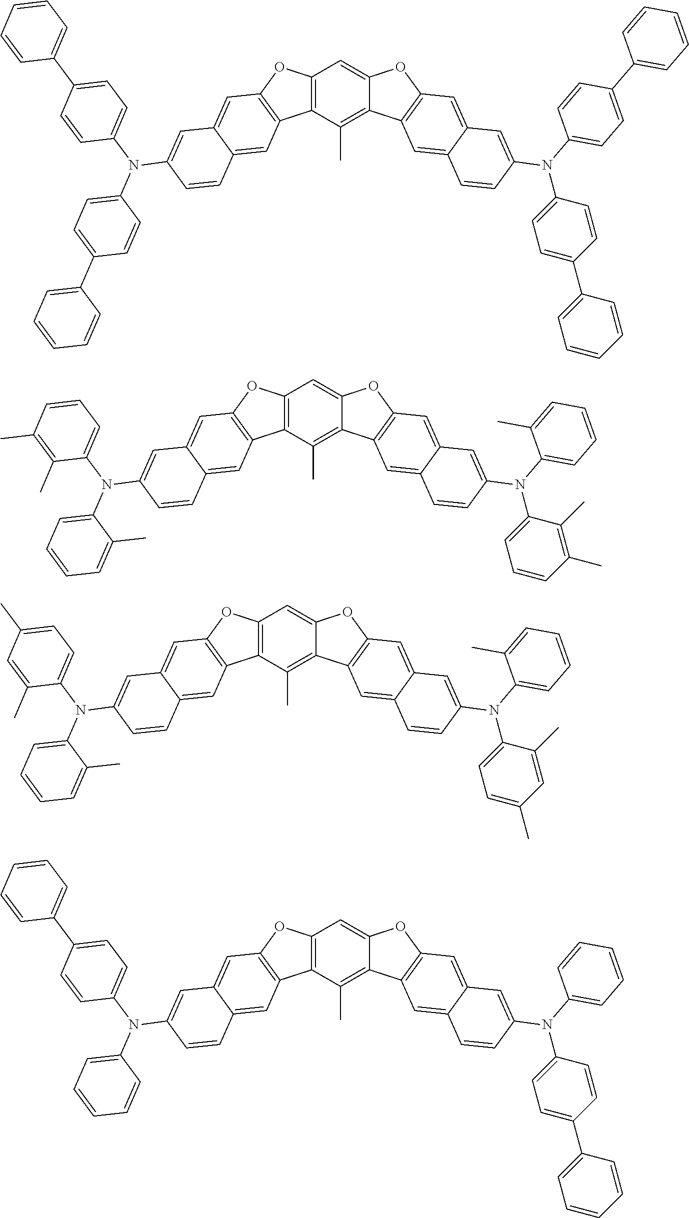

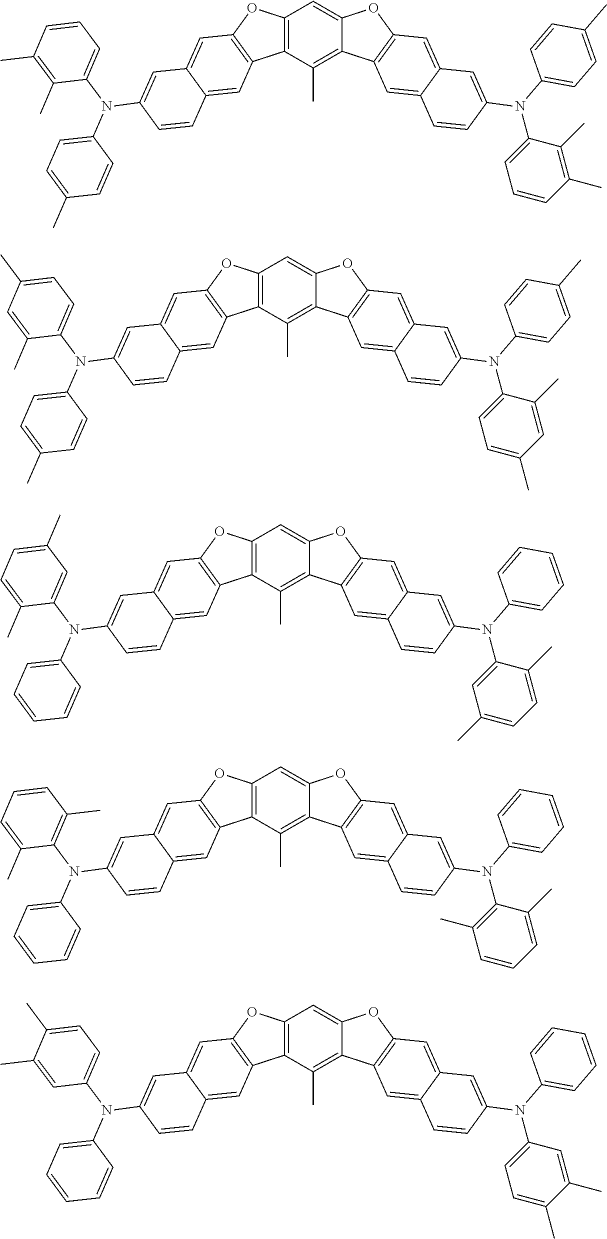

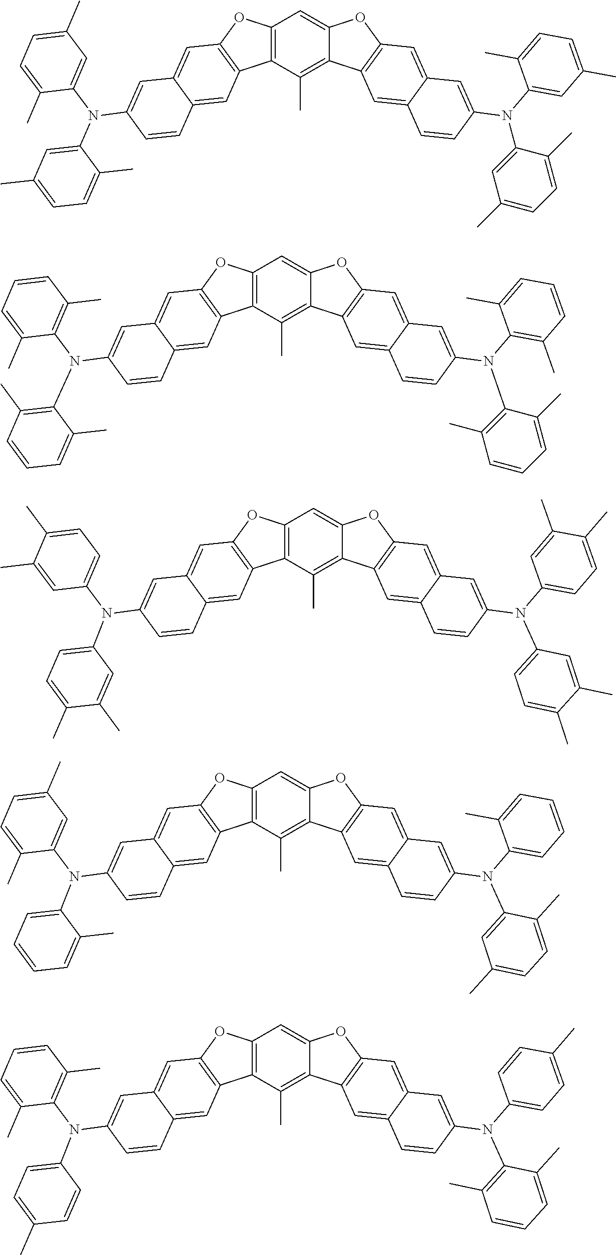

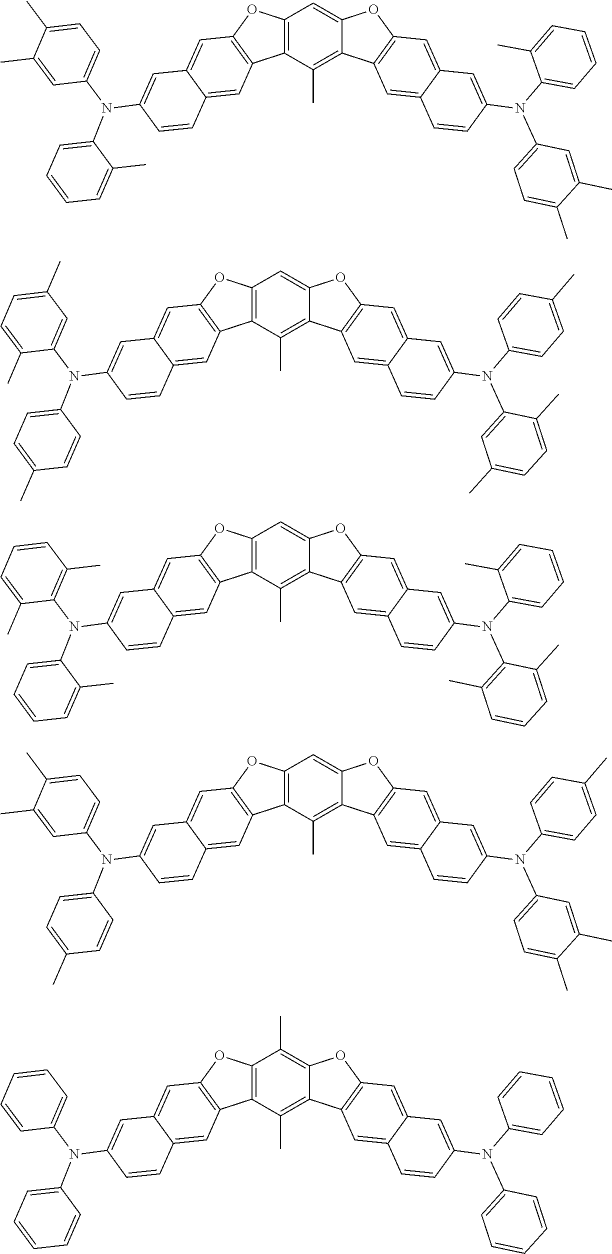

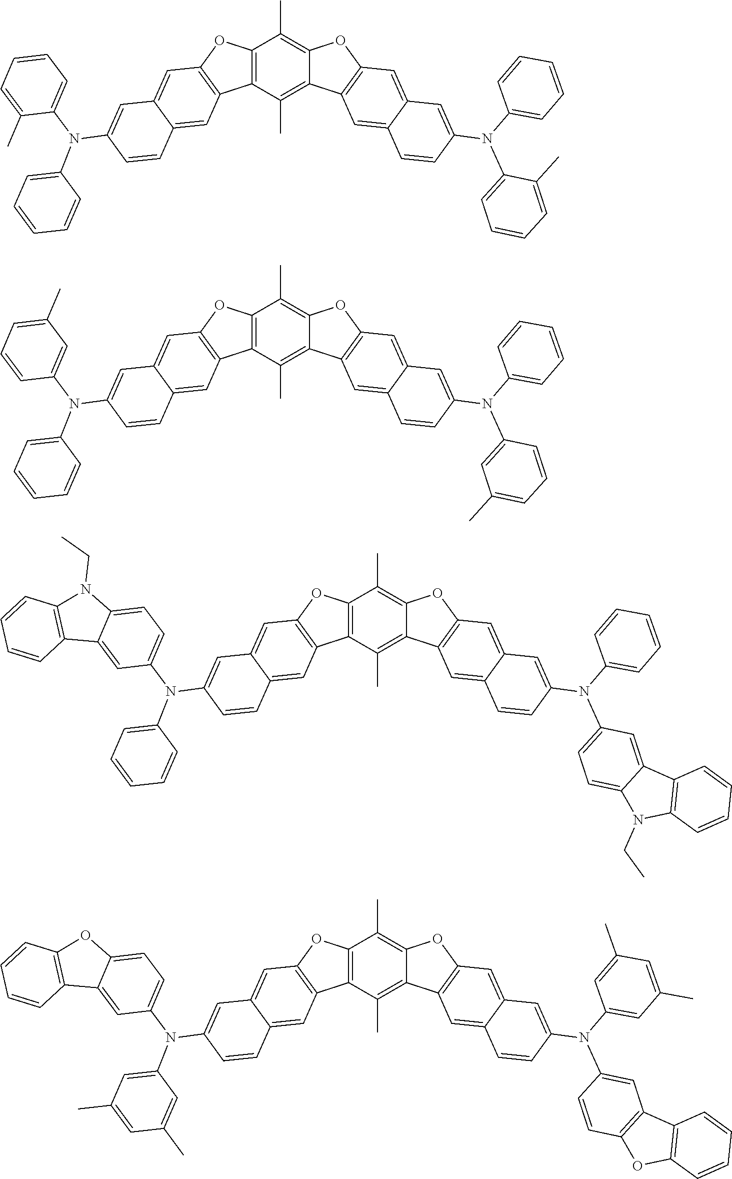

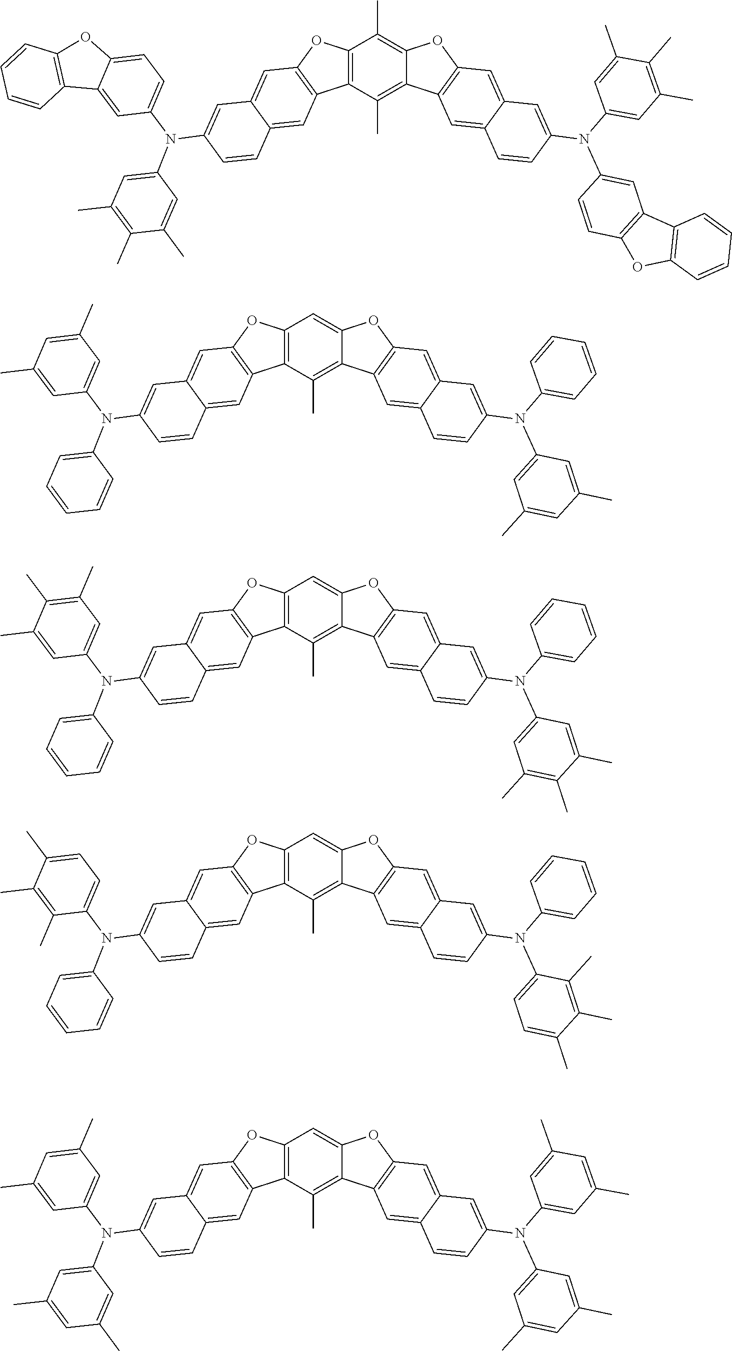

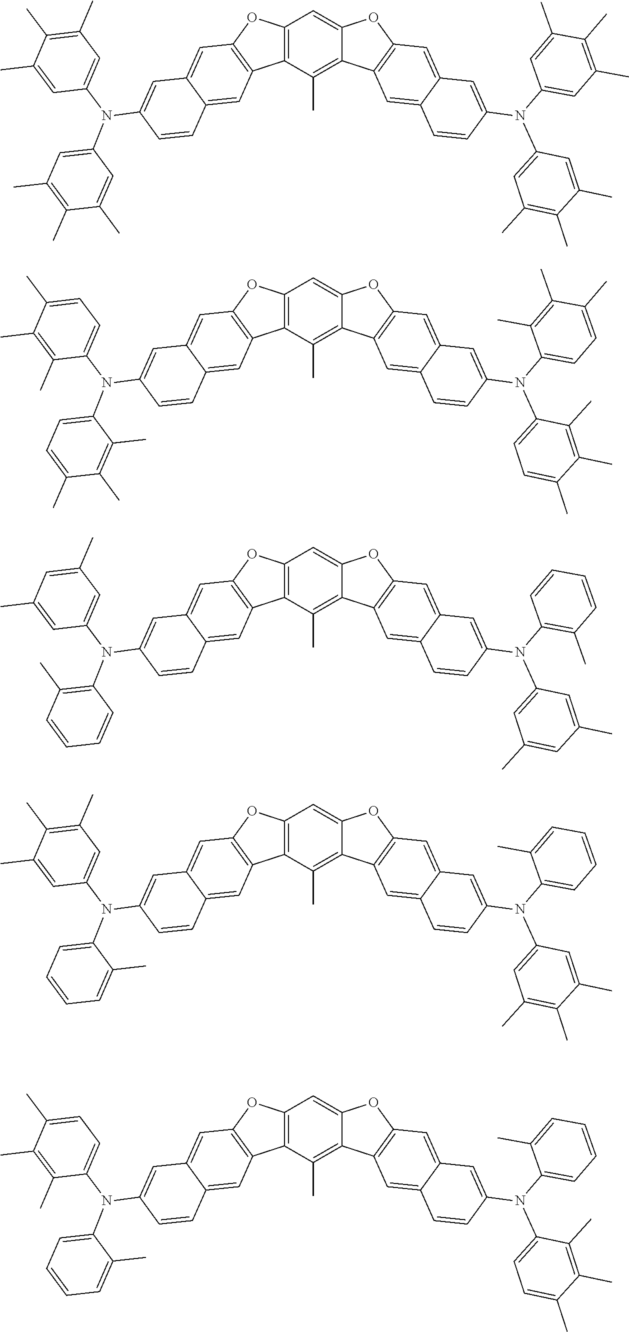

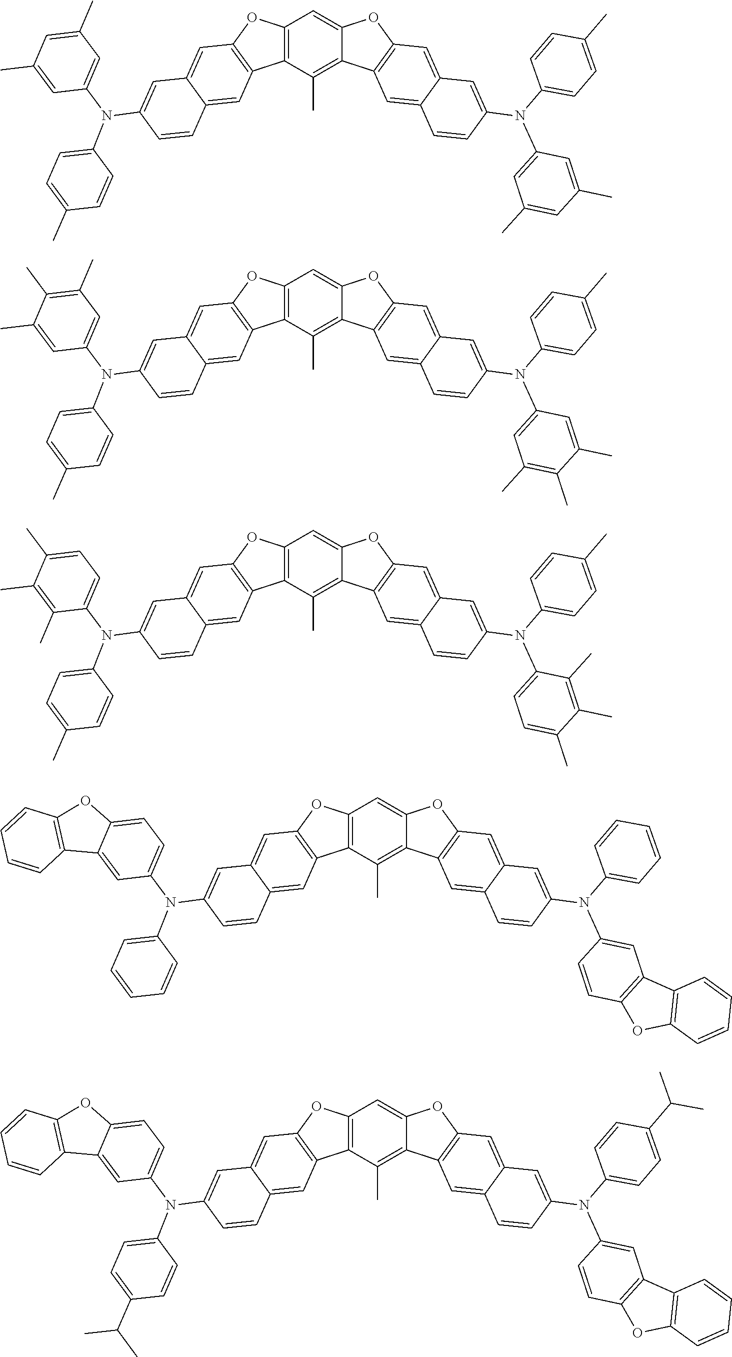

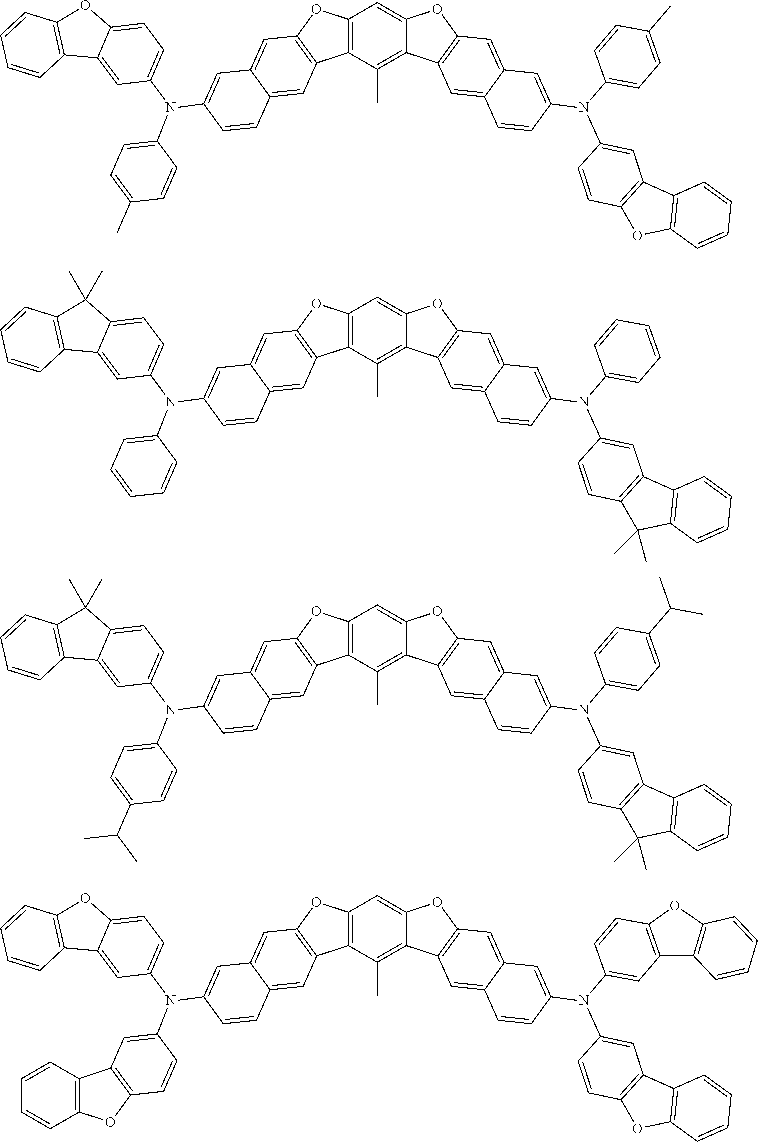

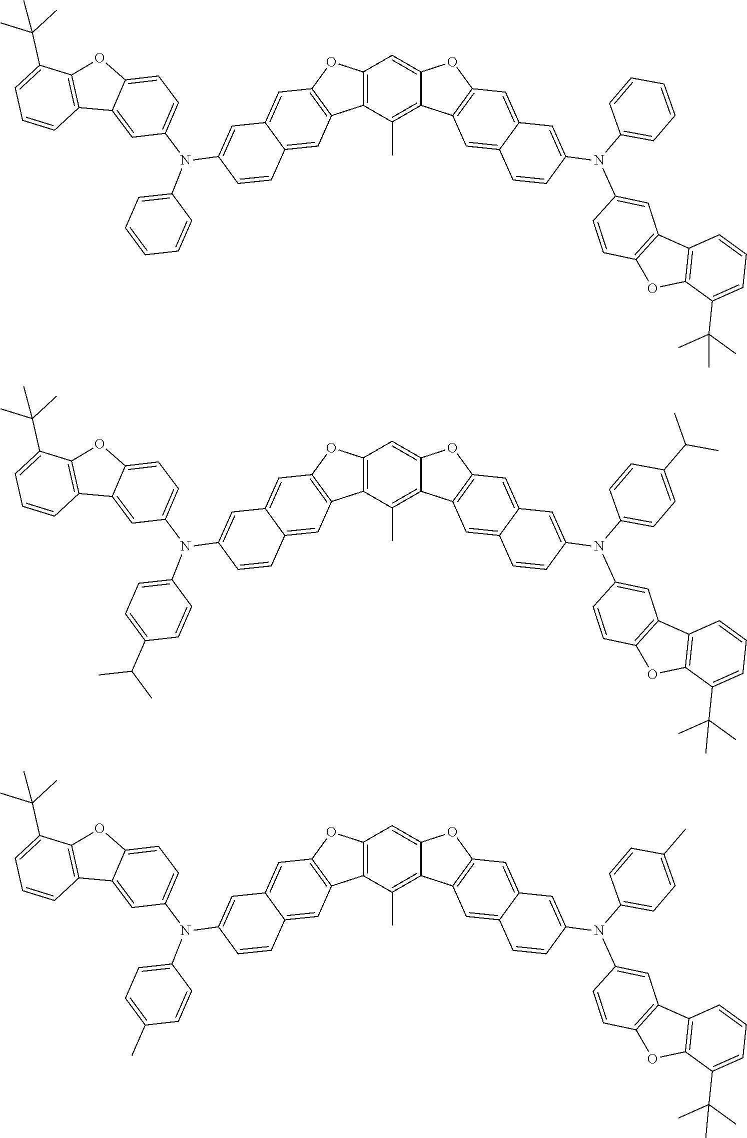 [SMILES: Cc1c2c(cc3oc4cc5cc(N(c6ccccc6)c6ccc7oc8c(C(C)(C)C)cccc8c7c6)ccc5cc4c13)oc1cc3cc(N(c4ccccc4)c4ccc5oc6c(C(C)(C)C)cccc6c5c4)ccc3cc12 ; Cc1c2c(cc3oc4cc5cc(N(c6ccc(C(C)C)cc6)c6ccc7oc8c(C(C)(C)C)cccc8c7c6)ccc5cc4c13)oc1cc3cc(N(c4ccc(C(C)C)cc4)c4ccc5oc6c(C(C)(C)C)cccc6c5c4)ccc3cc12 ; Cc1ccc(N(c2ccc3cc4c(cc3c2)oc2cc3oc5cc6cc(N(c7ccc(C)cc7)c7ccc8oc9c(C(C)(C)C)cccc9c8c7)ccc6cc5c3c(C)c24)c2ccc3oc4c(C(C)(C)C)cccc4c3c2)cc1]

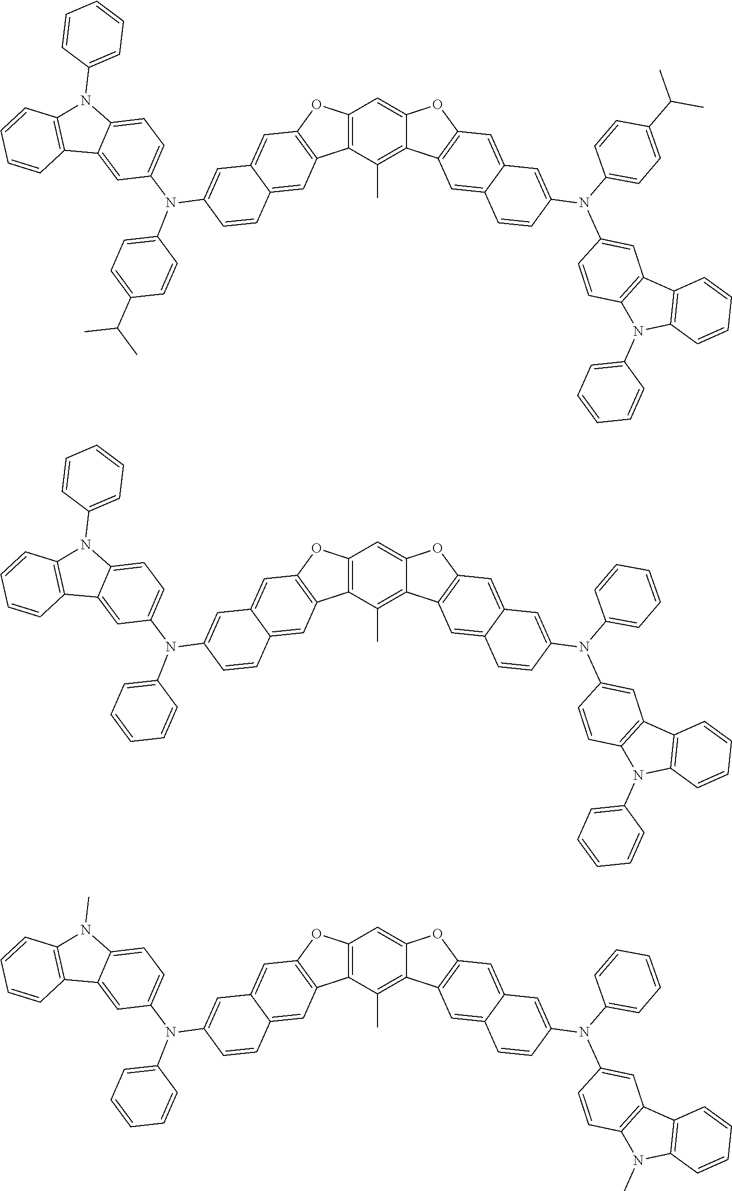 [SMILES: Cc1c2c(cc3oc4cc5cc(N(c6ccc(C(C)C)cc6)c6ccc7c(c6)c6ccccc6n7-c6ccccc6)ccc5cc4c13)oc1cc3cc(N(c4ccc(C(C)C)cc4)c4ccc5c(c4)c4ccccc4n5-c4ccccc4)ccc3cc12 ; Cc1c2c(cc3oc4cc5cc(N(c6ccccc6)c6ccc7c(c6)c6ccccc6n7-c6ccccc6)ccc5cc4c13)oc1cc3cc(N(c4ccccc4)c4ccc5c(c4)c4ccccc4n5-c4ccccc4)ccc3cc12 ; Cc1c2c(cc3oc4cc5cc(N(c6ccccc6)c6ccc7c(c6)c6ccccc6n7C)ccc5cc4c13)oc1cc3cc(N(c4ccccc4)c4ccc5c(c4)c4ccccc4n5C)ccc3cc12]

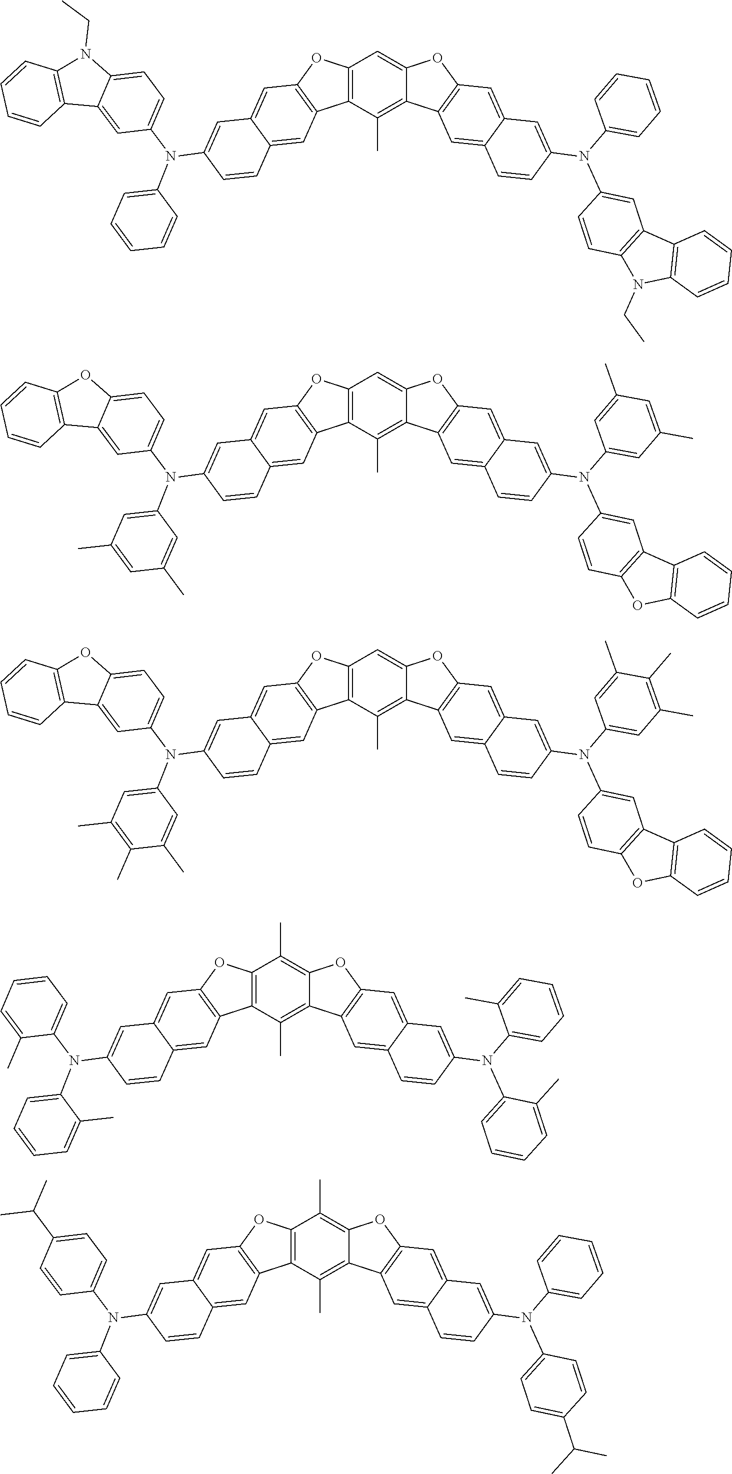

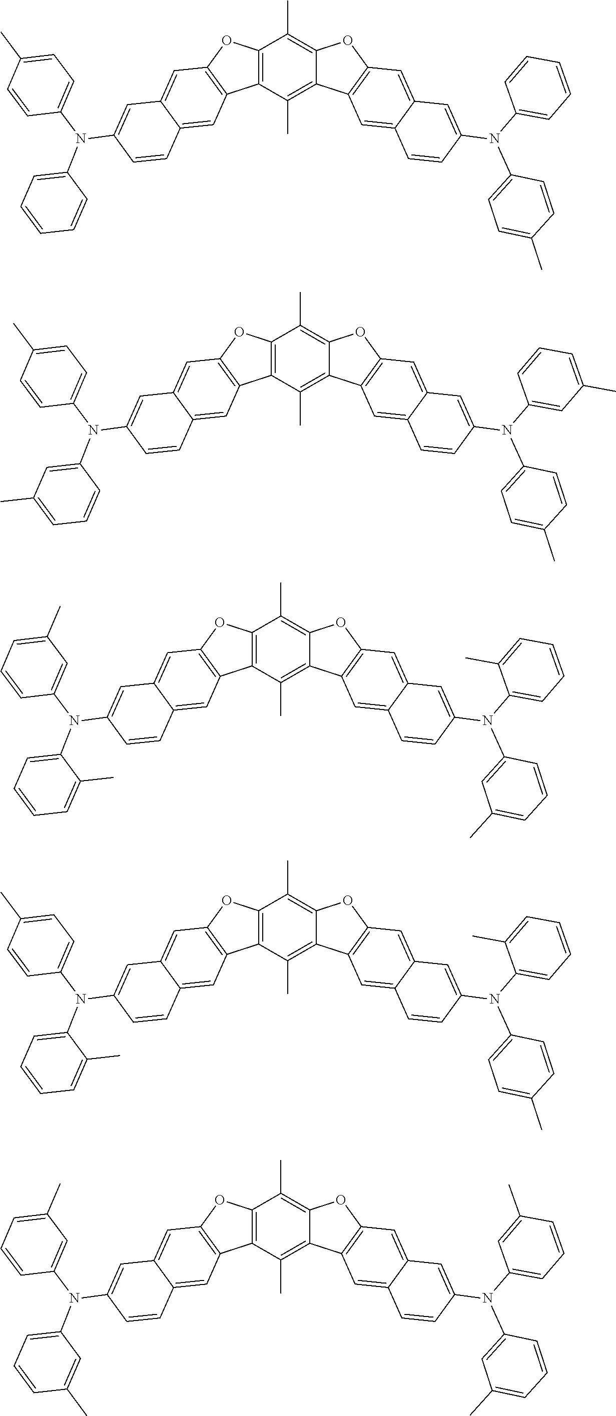

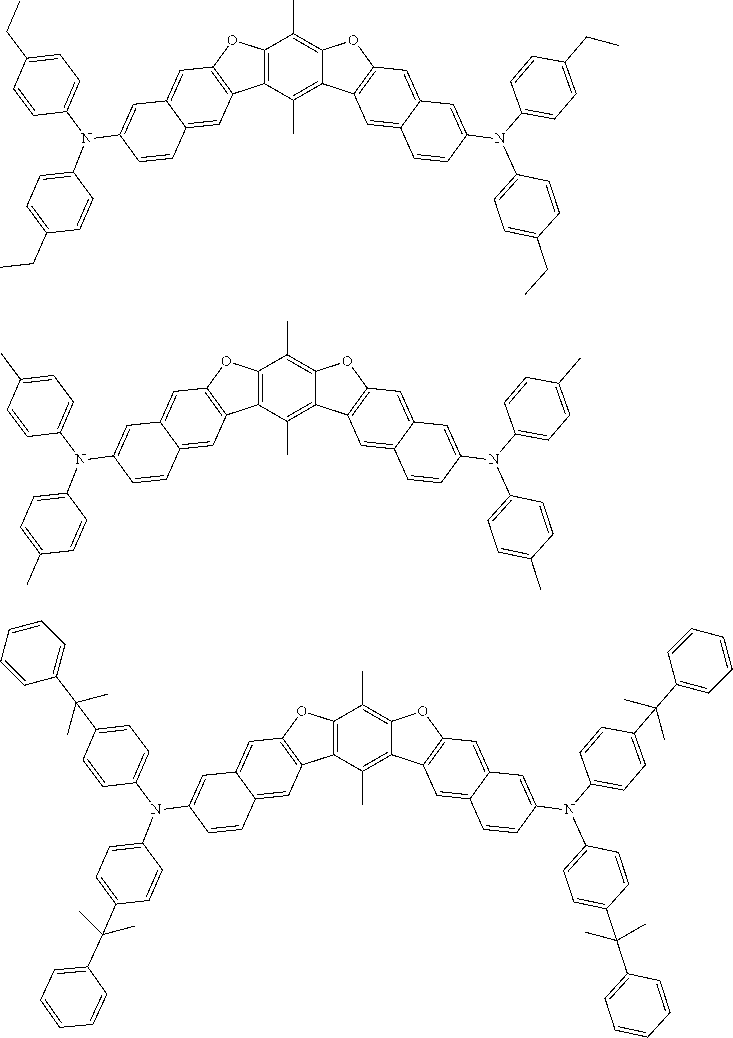

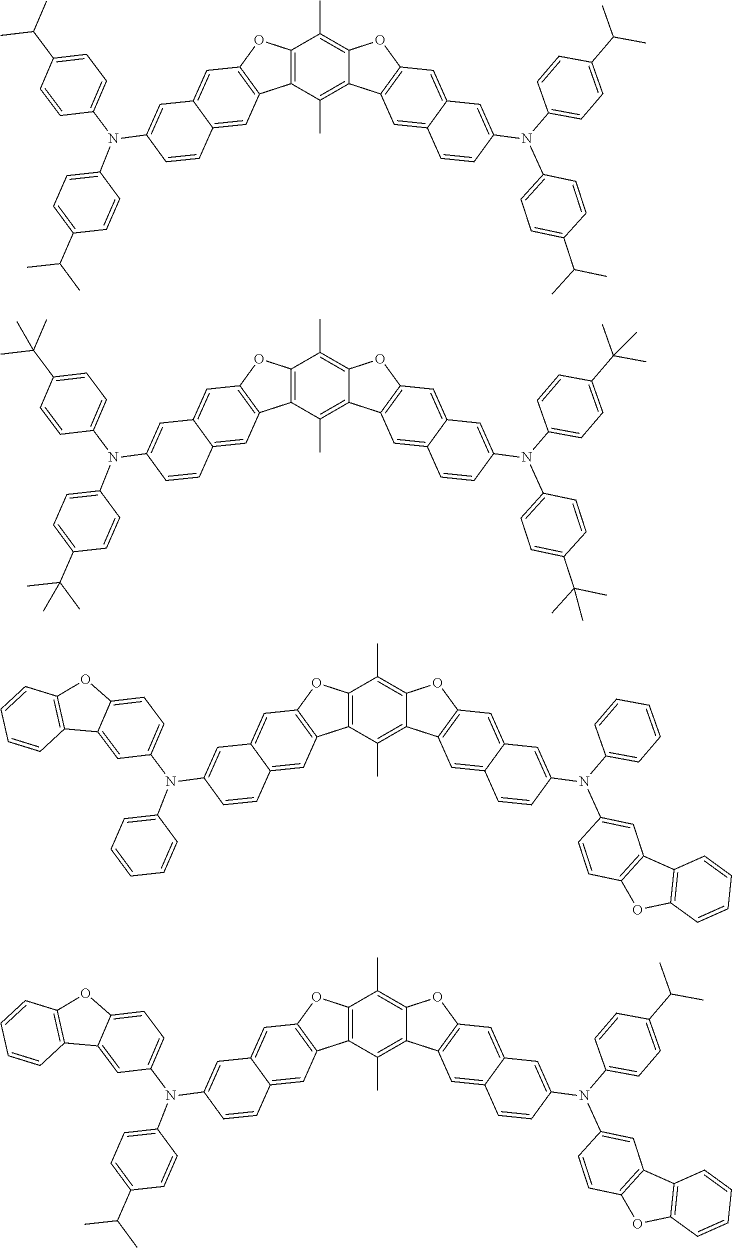

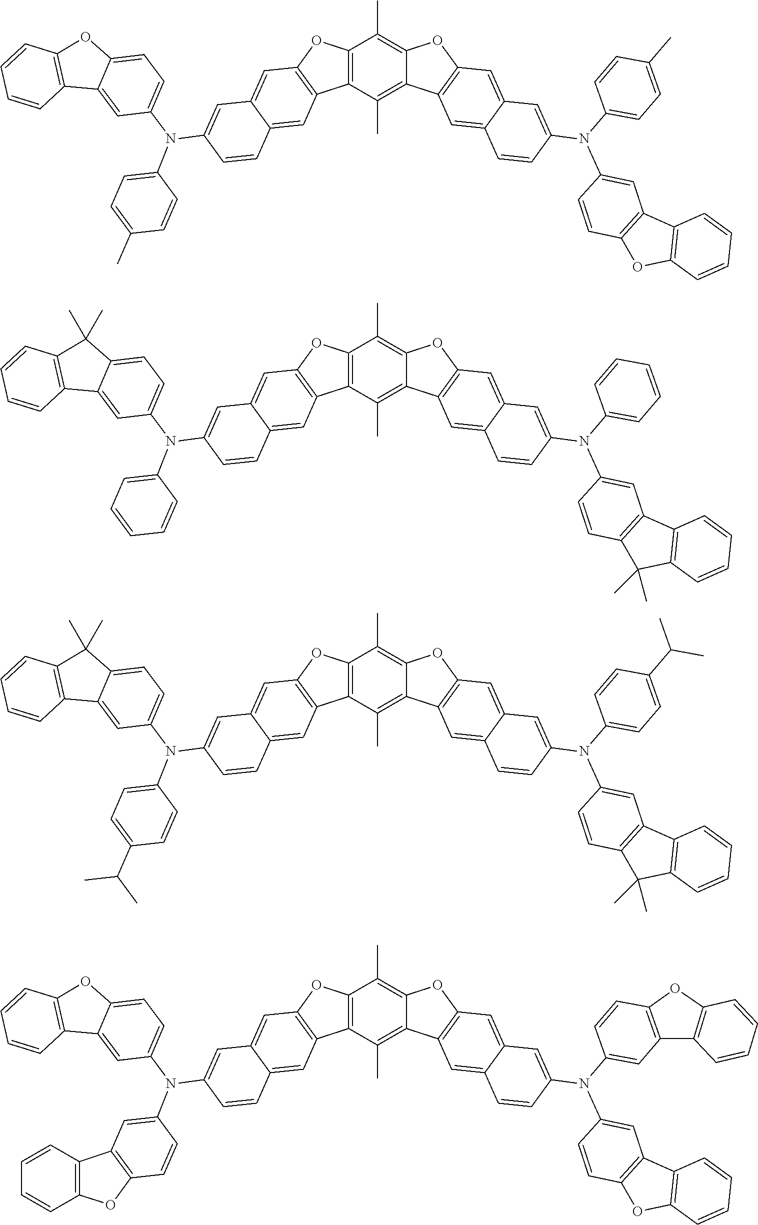

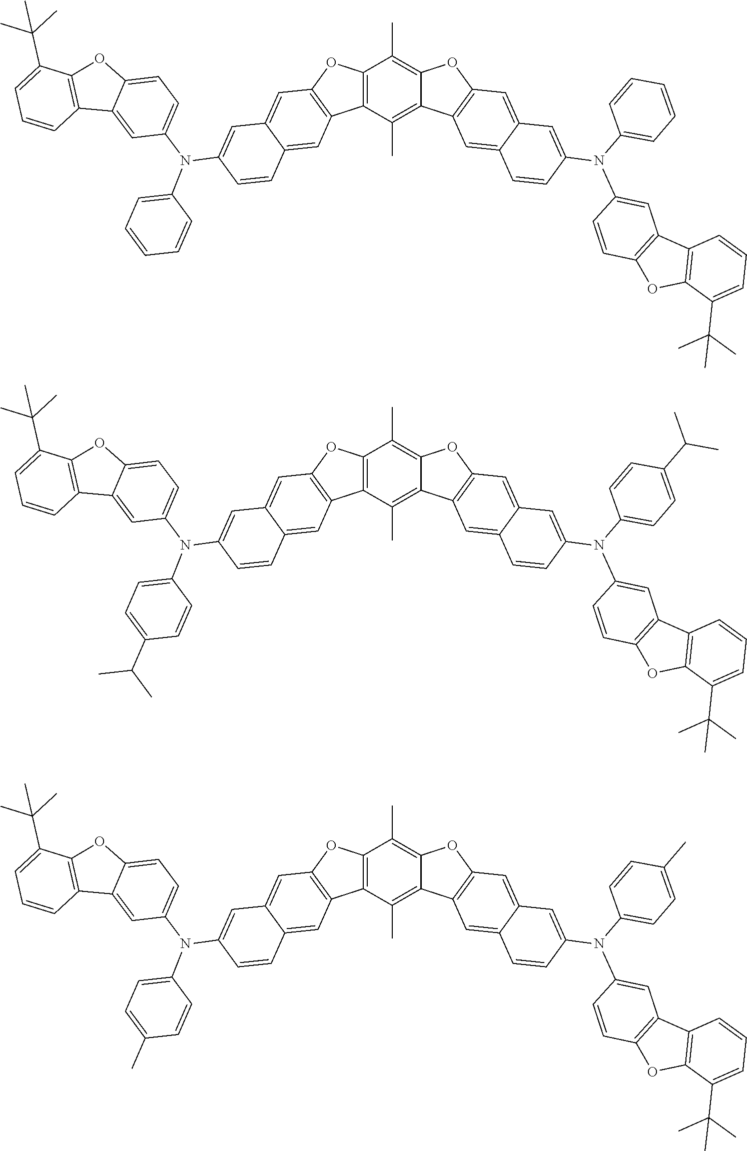 [SMILES: Cc1c2oc3cc4cc(N(c5ccccc5)c5ccc6oc7c(C(C)(C)C)cccc7c6c5)ccc4cc3c2c(C)c2c1oc1cc3cc(N(c4ccccc4)c4ccc5oc6c(C(C)(C)C)cccc6c5c4)ccc3cc12 ; Cc1c2oc3cc4cc(N(c5ccc(C(C)C)cc5)c5ccc6oc7c(C(C)(C)C)cccc7c6c5)ccc4cc3c2c(C)c2c1oc1cc3cc(N(c4ccc(C(C)C)cc4)c4ccc5oc6c(C(C)(C)C)cccc6c5c4)ccc3cc12 ; Cc1ccc(N(c2ccc3cc4c(cc3c2)oc2c(C)c3oc5cc6cc(N(c7ccc(C)cc7)c7ccc8oc9c(C(C)(C)C)cccc9c8c7)ccc6cc5c3c(C)c24)c2ccc3oc4c(C(C)(C)C)cccc4c3c2)cc1]

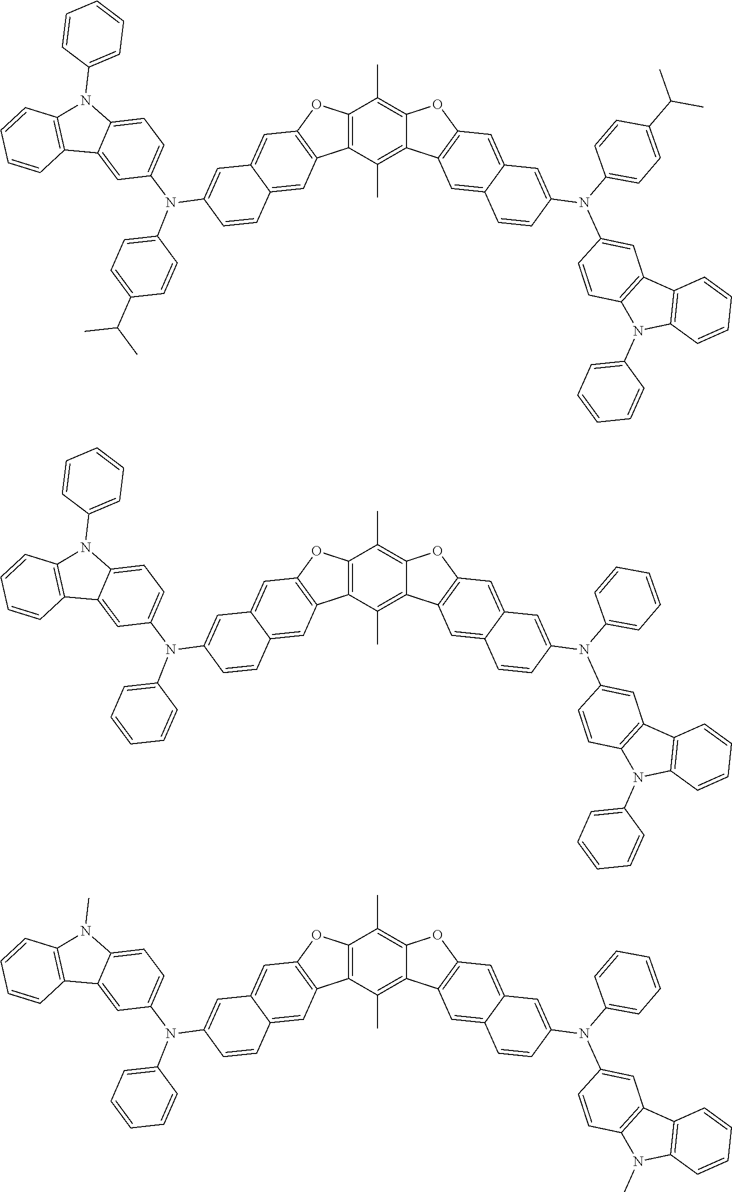 [SMILES: Cc1c2oc3cc4cc(N(c5ccc(C(C)C)cc5)c5ccc6c(c5)c5ccccc5n6-c5ccccc5)ccc4cc3c2c(C)c2c1oc1cc3cc(N(c4ccc(C(C)C)cc4)c4ccc5c(c4)c4ccccc4n5-c4ccccc4)ccc3cc12 ; Cc1c2oc3cc4cc(N(c5ccccc5)c5ccc6c(c5)c5ccccc5n6-c5ccccc5)ccc4cc3c2c(C)c2c1oc1cc3cc(N(c4ccccc4)c4ccc5c(c4)c4ccccc4n5-c4ccccc4)ccc3cc12 ; Cc1c2oc3cc4cc(N(c5ccccc5)c5ccc6c(c5)c5ccccc5n6C)ccc4cc3c2c(C)c2c1oc1cc3cc(N(c4ccccc4)c4ccc5c(c4)c4ccccc4n5C)ccc3cc12]

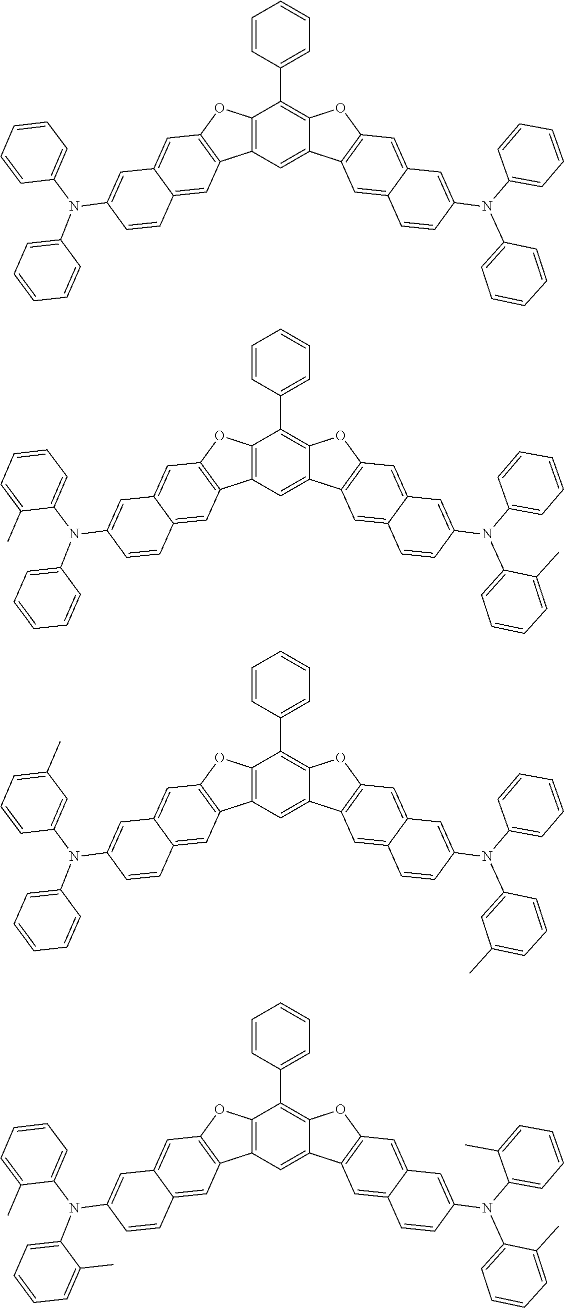 [SMILES: c1ccc(-c2c3oc4cc5cc(N(c6ccccc6)c6ccccc6)ccc5cc4c3cc3c2oc2cc4cc(N(c5ccccc5)c5ccccc5)ccc4cc23)cc1 ; Cc1ccccc1N(c1ccccc1)c1ccc2cc3c(cc2c1)oc1c(-c2ccccc2)c2oc4cc5cc(N(c6ccccc6)c6ccccc6C)ccc5cc4c2cc13 ; Cc1cccc(N(c2ccccc2)c2ccc3cc4c(cc3c2)oc2c(-c3ccccc3)c3oc5cc6cc(N(c7ccccc7)c7cccc(C)c7)ccc6cc5c3cc24)c1 ; Cc1ccccc1N(c1ccc2cc3c(cc2c1)oc1c(-c2ccccc2)c2oc4cc5cc(N(c6ccccc6C)c6ccccc6C)ccc5cc4c2cc13)c1ccccc1C]

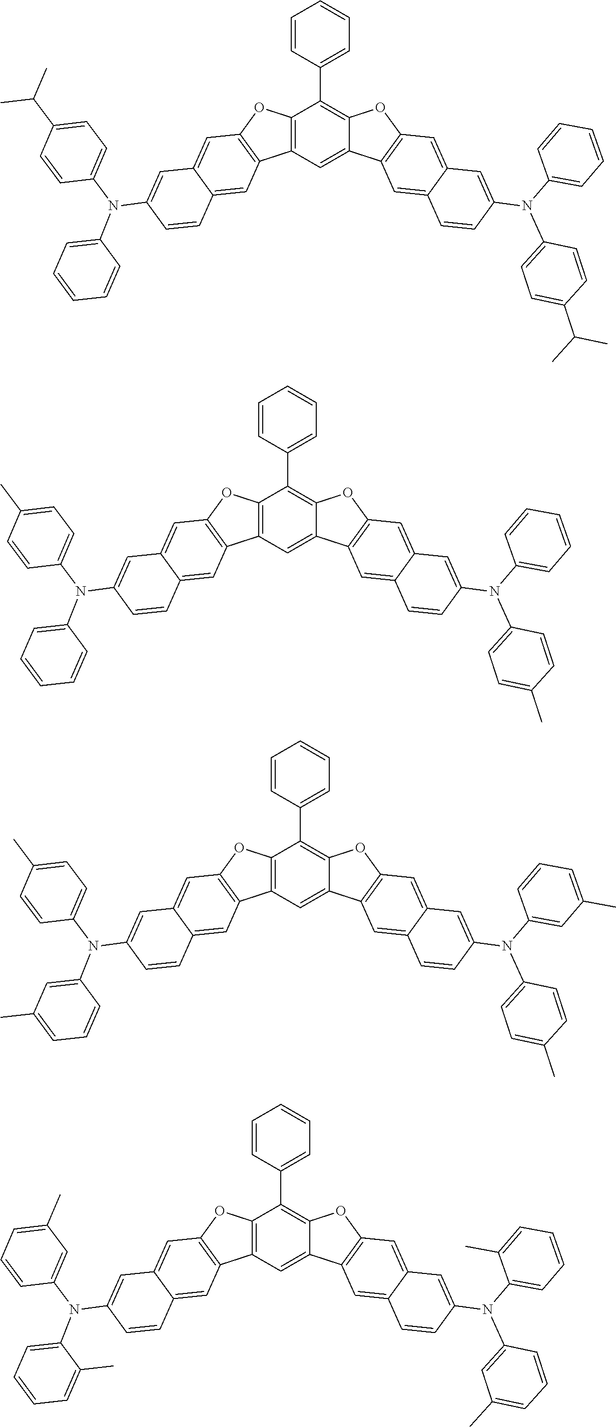

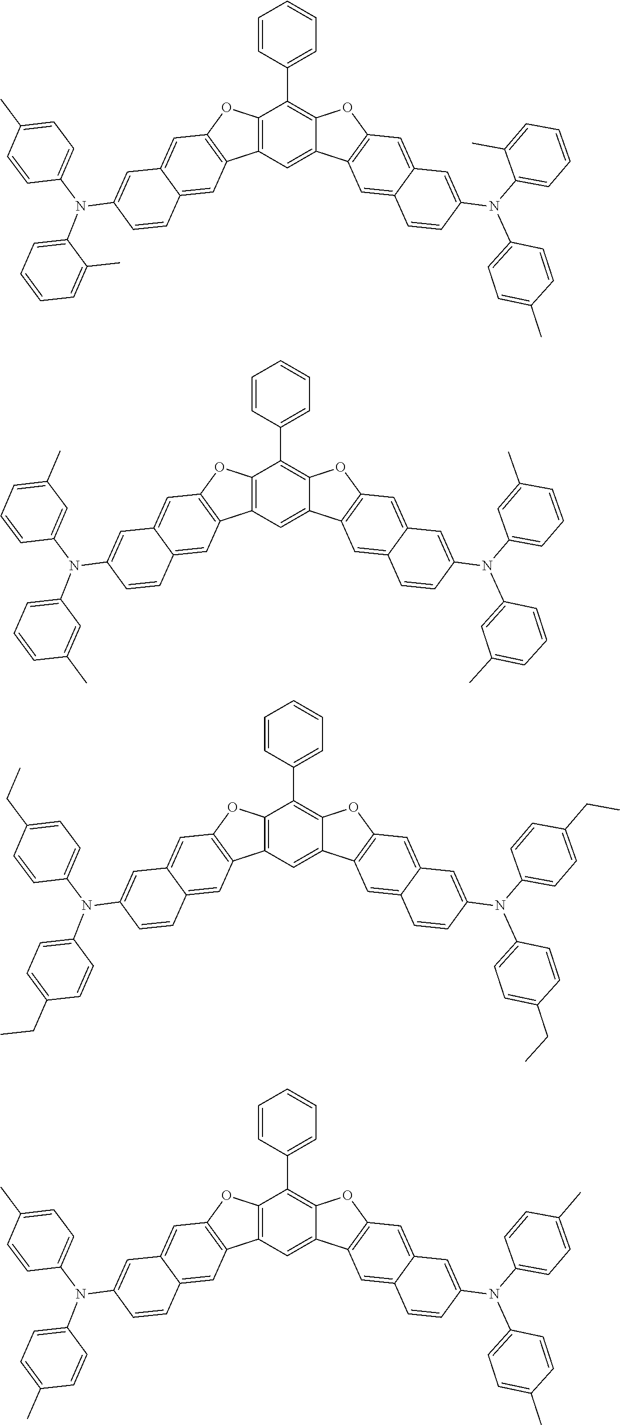

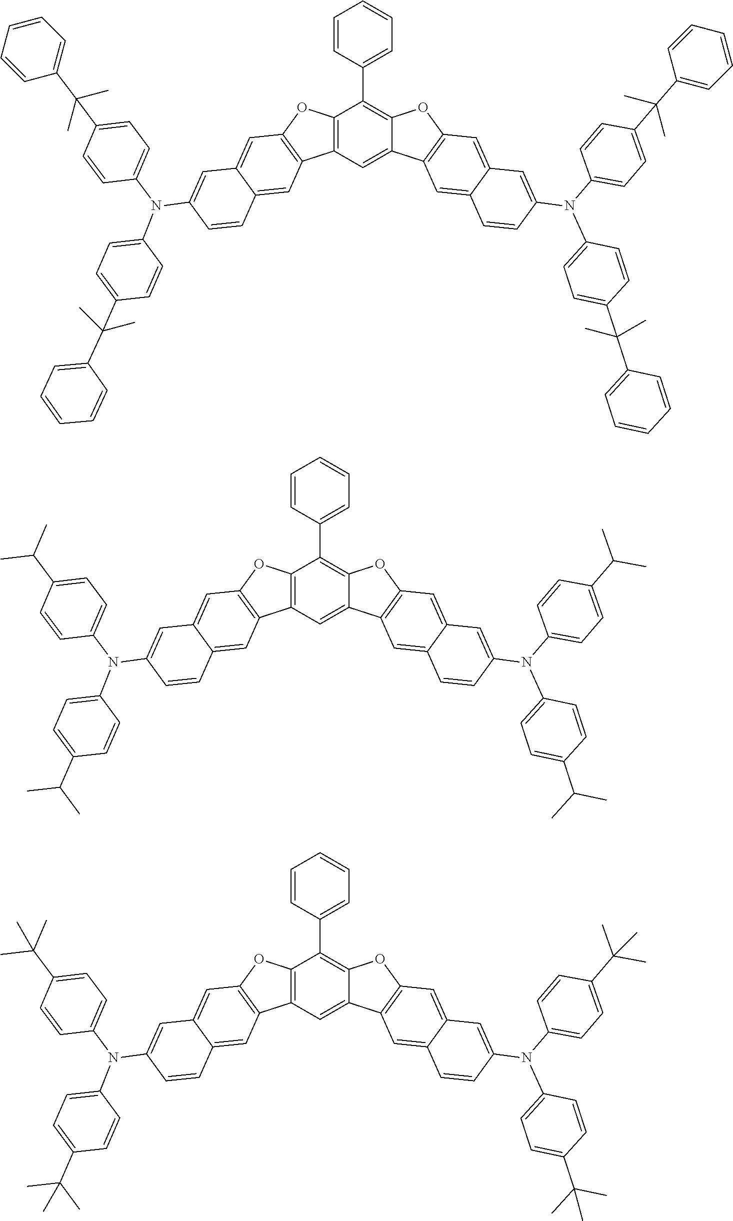

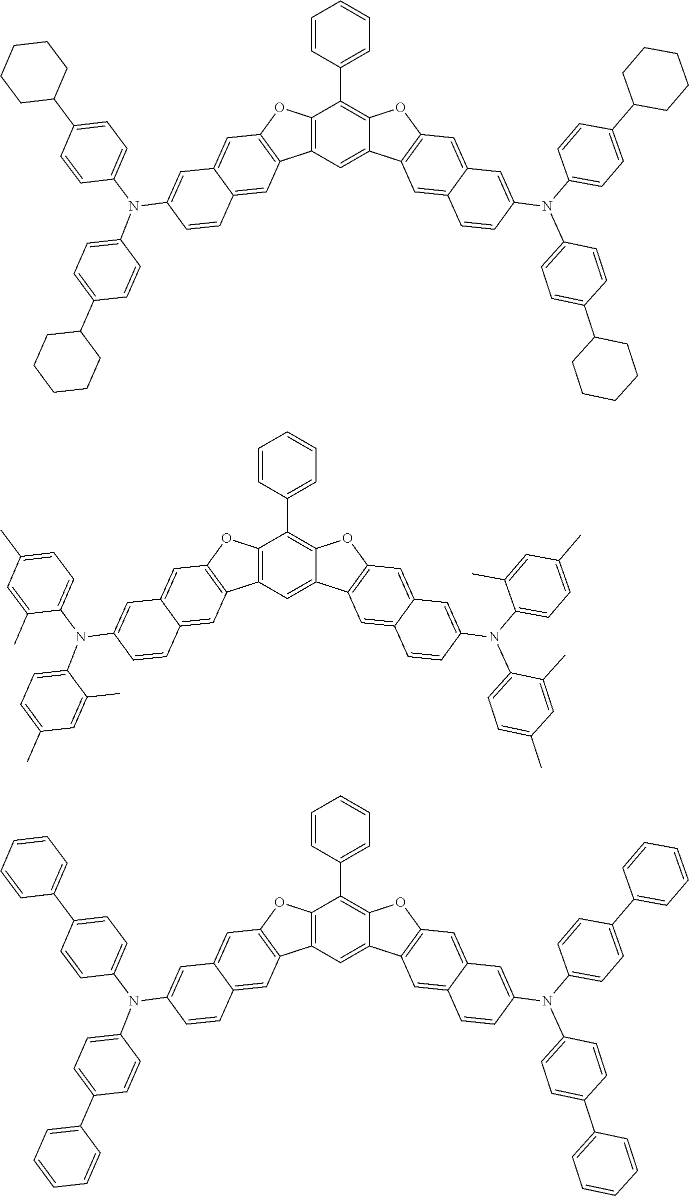

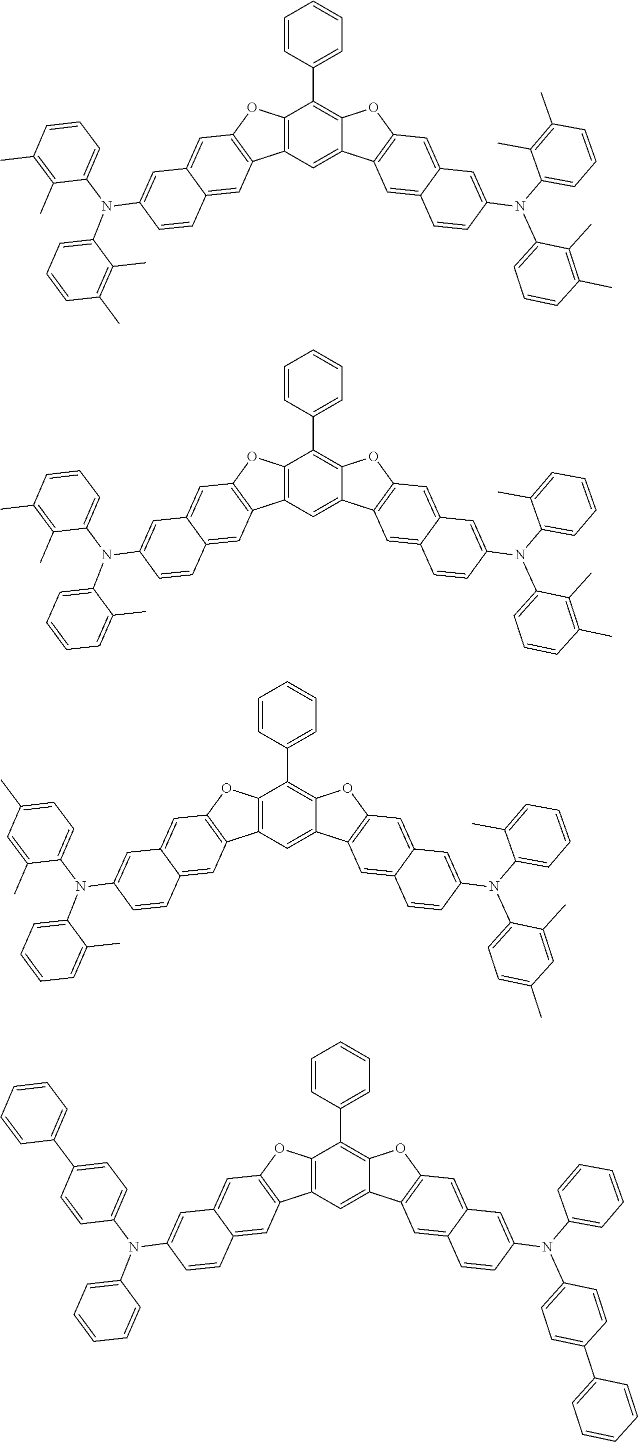

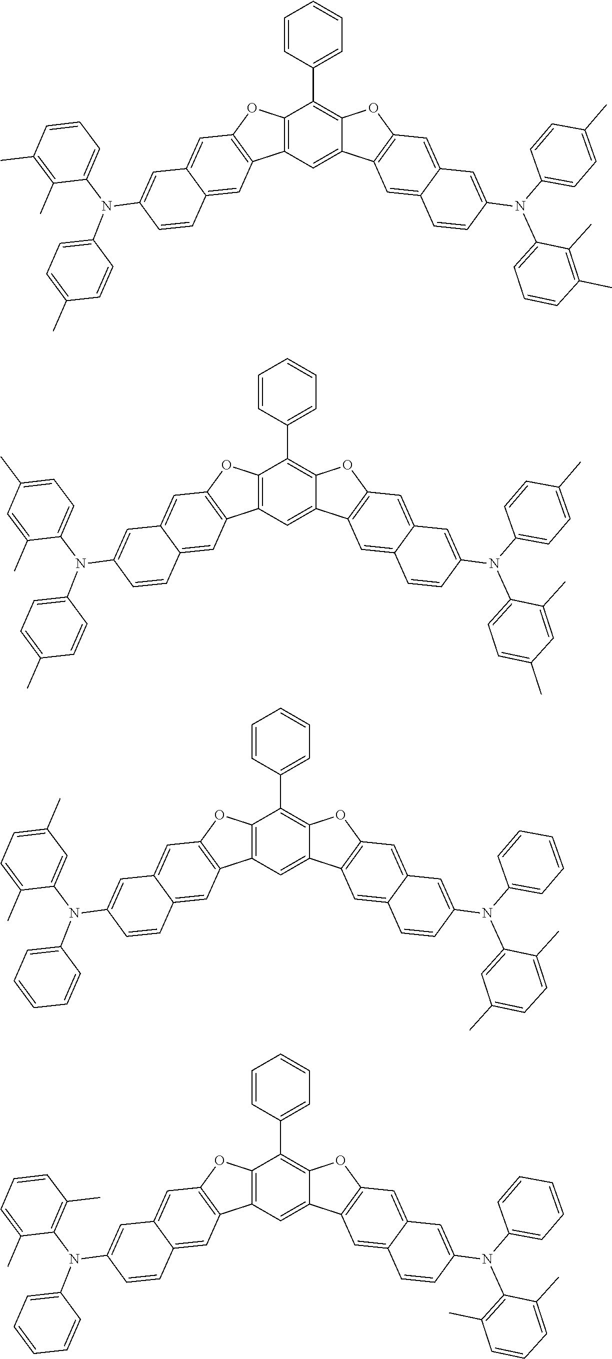

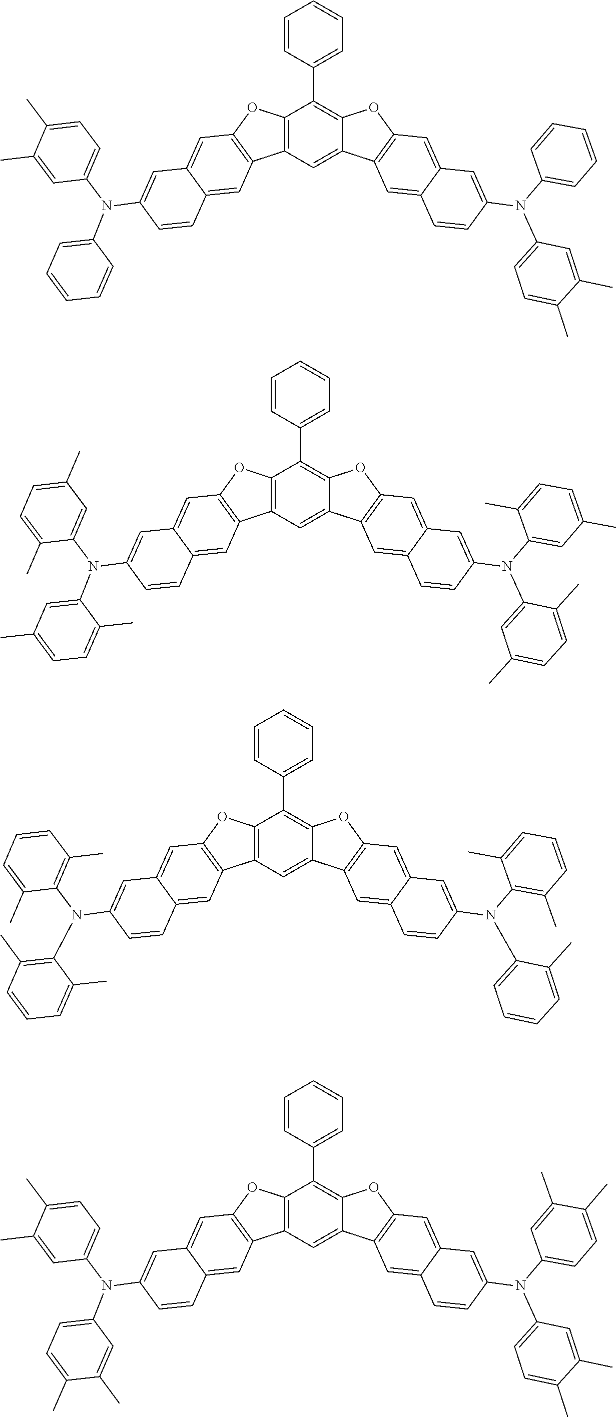

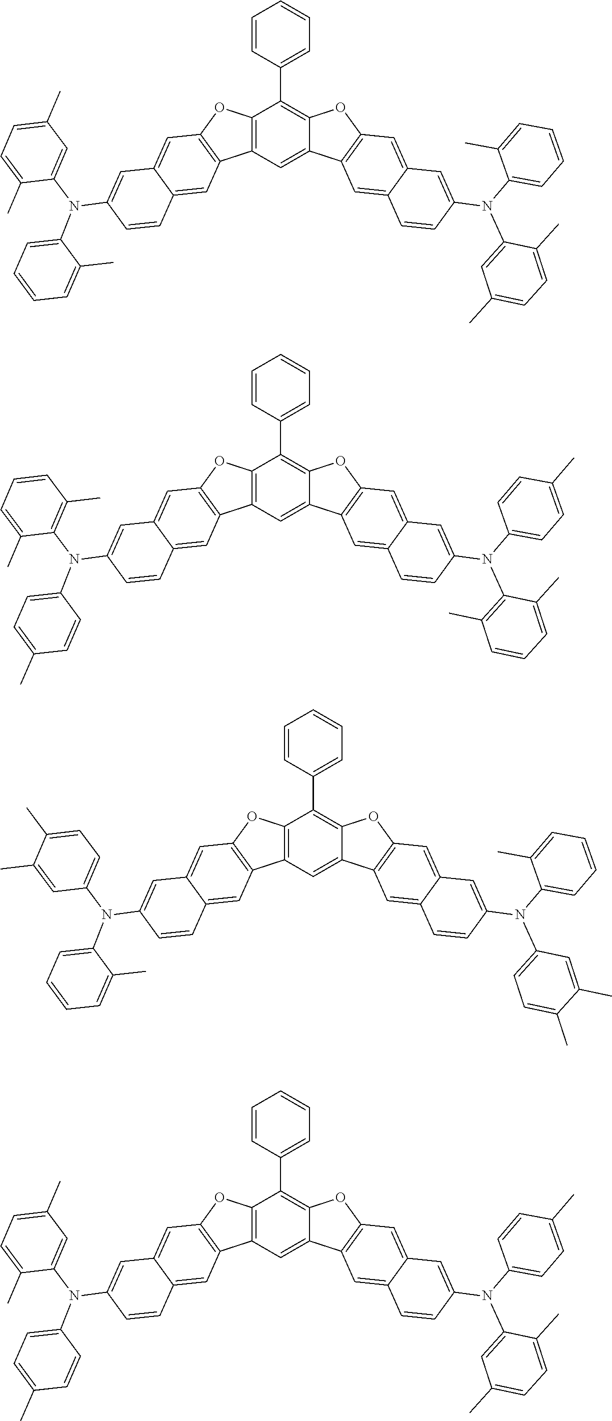

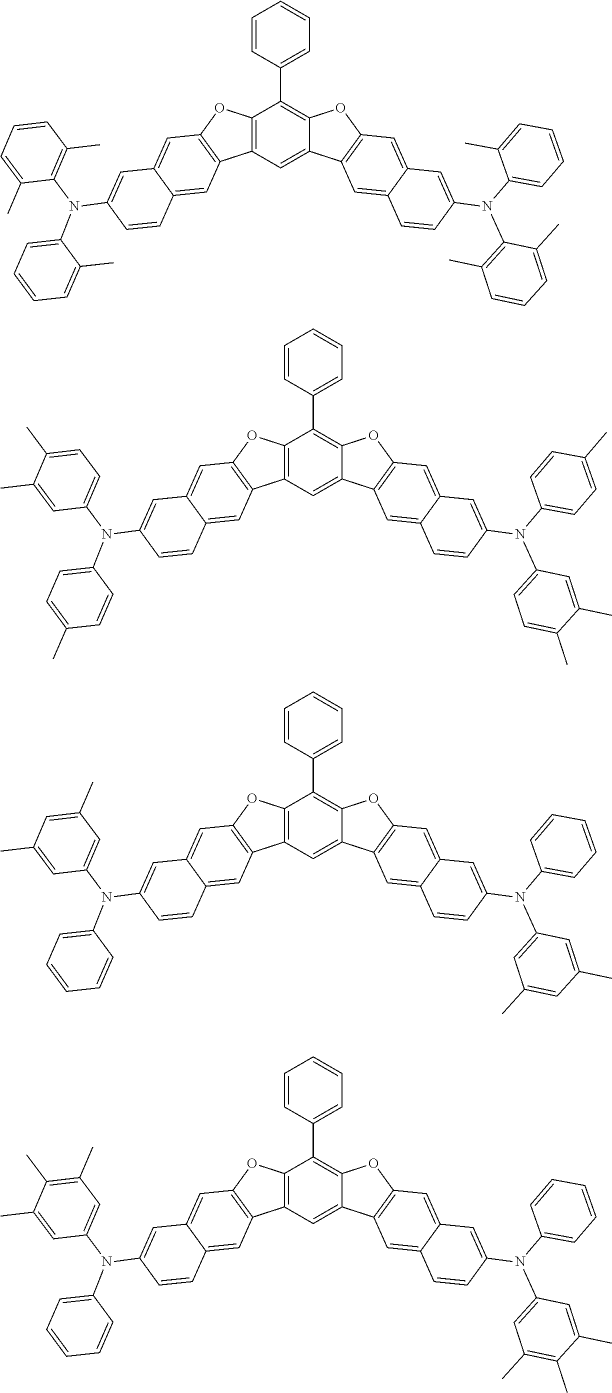

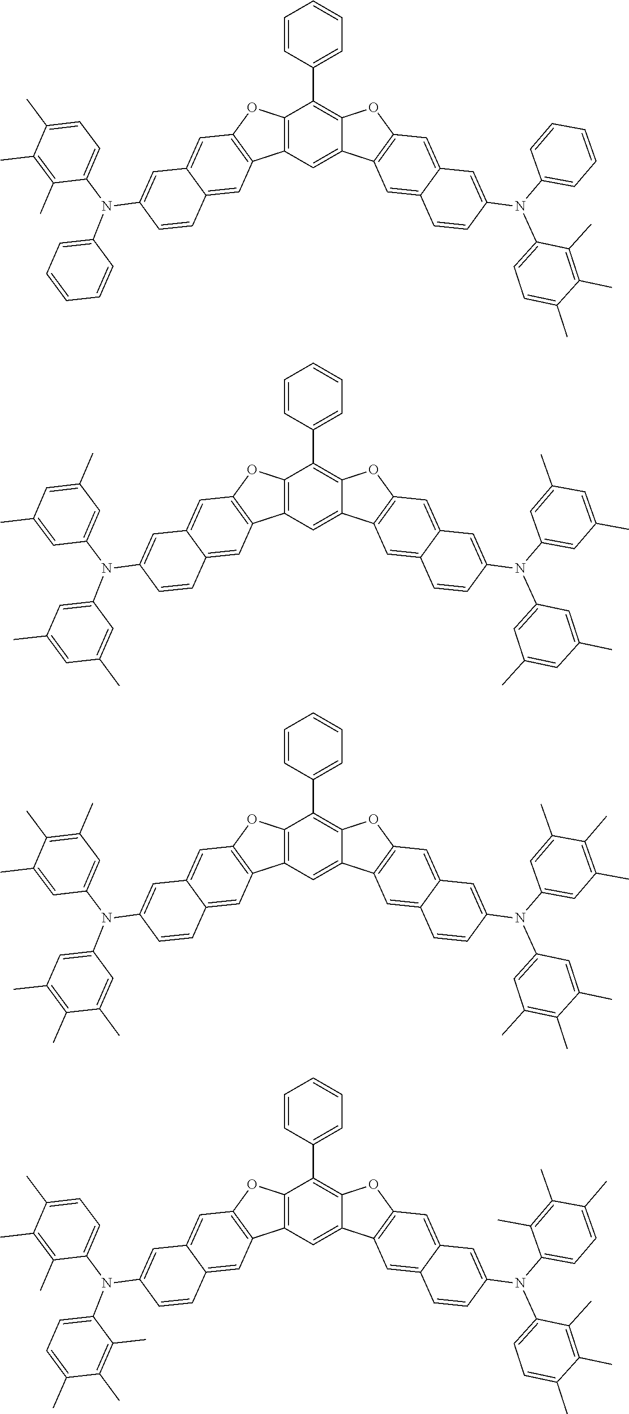

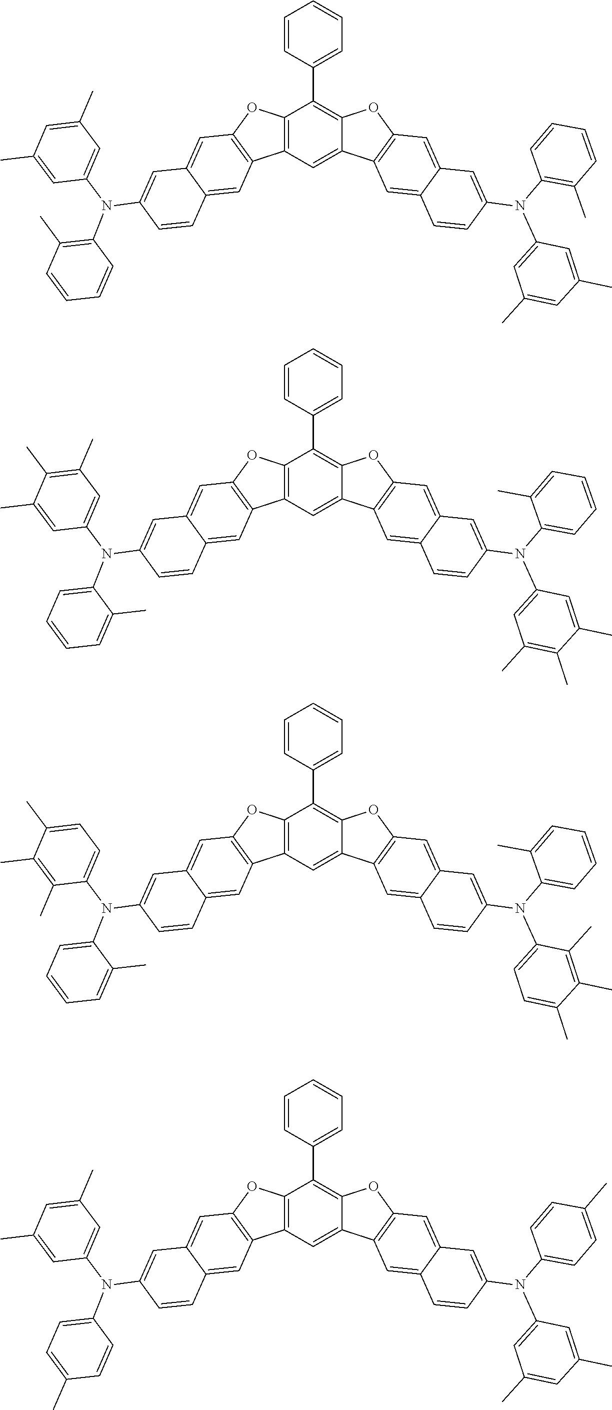

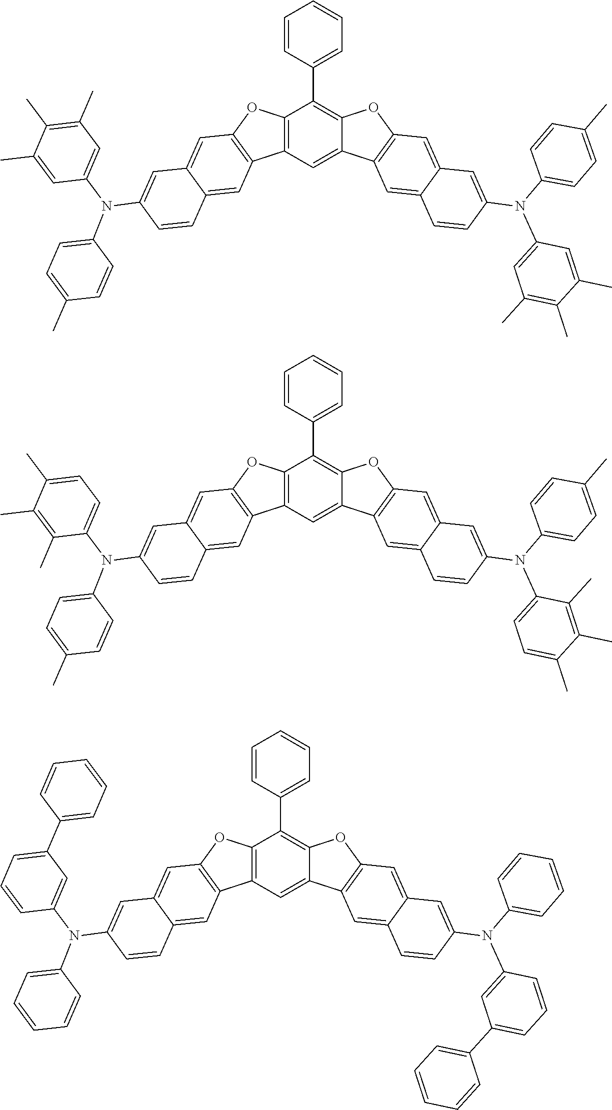

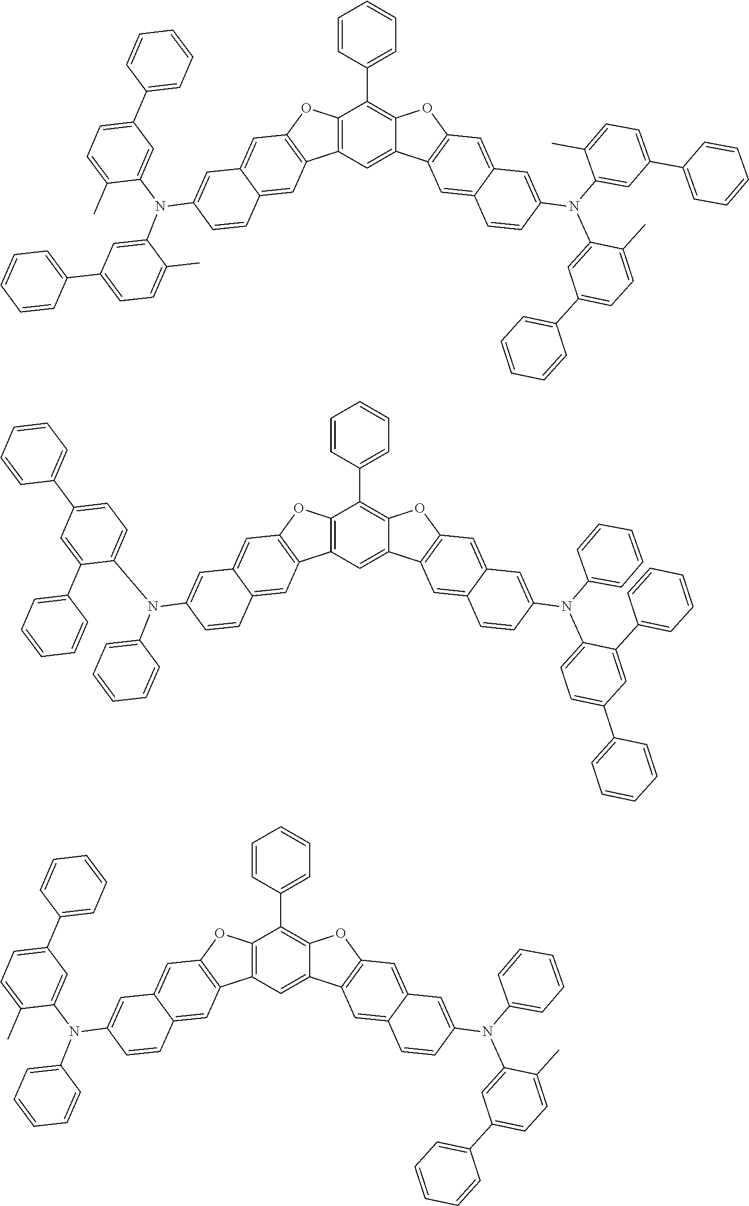 [SMILES: Cc1ccc(-c2ccccc2)cc1N(c1ccc2cc3c(cc2c1)oc1c(-c2ccccc2)c2oc4cc5cc(N(c6cc(-c7ccccc7)ccc6C)c6cc(-c7ccccc7)ccc6C)ccc5cc4c2cc13)c1cc(-c2ccccc2)ccc1C ; c1ccc(-c2ccc(N(c3ccccc3)c3ccc4cc5c(cc4c3)oc3c(-c4ccccc4)c4oc6cc7cc(N(c8ccccc8)c8ccc(-c9ccccc9)cc8-c8ccccc8)ccc7cc6c4cc35)c(-c3ccccc3)c2)cc1 ; Cc1ccc(-c2ccccc2)cc1N(c1ccccc1)c1ccc2cc3c(cc2c1)oc1c(-c2ccccc2)c2oc4cc5cc(N(c6ccccc6)c6cc(-c7ccccc7)ccc6C)ccc5cc4c2cc13]

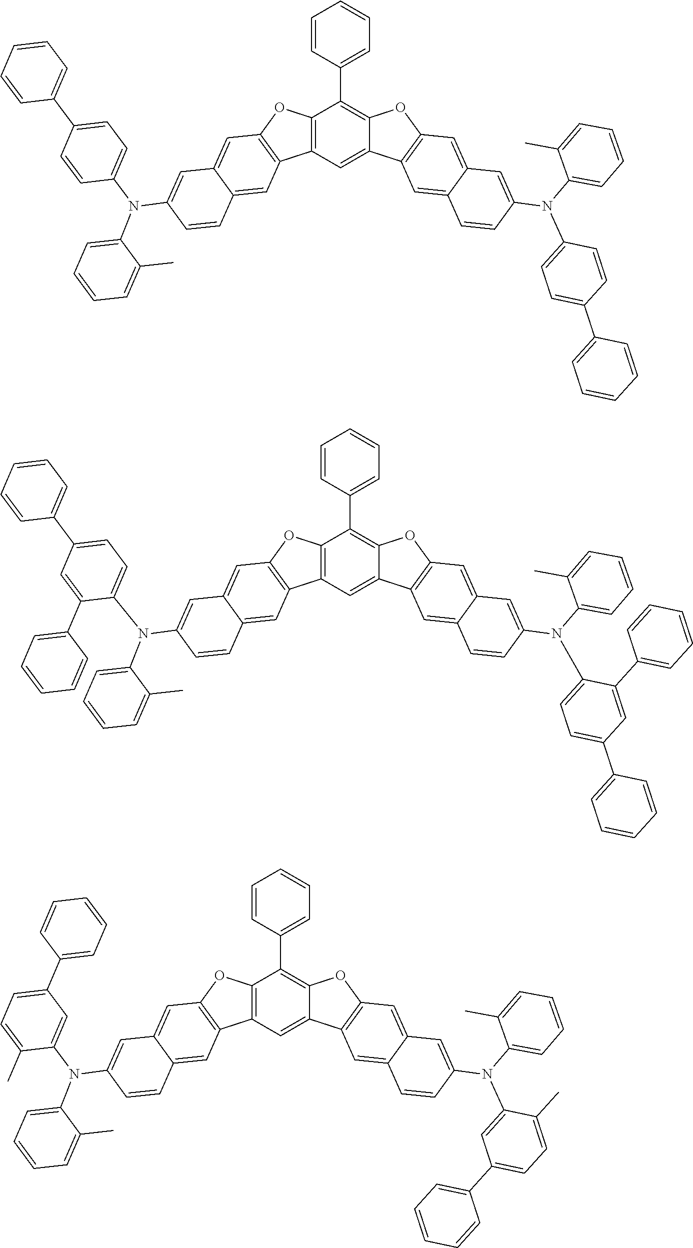 [SMILES: Cc1ccccc1N(c1ccc(-c2ccccc2)cc1)c1ccc2cc3c(cc2c1)oc1c(-c2ccccc2)c2oc4cc5cc(N(c6ccc(-c7ccccc7)cc6)c6ccccc6C)ccc5cc4c2cc13 ; Cc1ccccc1N(c1ccc2cc3c(cc2c1)oc1c(-c2ccccc2)c2oc4cc5cc(N(c6ccccc6C)c6ccc(-c7ccccc7)cc6-c6ccccc6)ccc5cc4c2cc13)c1ccc(-c2ccccc2)cc1-c1ccccc1 ; Cc1ccccc1N(c1ccc2cc3c(cc2c1)oc1c(-c2ccccc2)c2oc4cc5cc(N(c6ccccc6C)c6cc(-c7ccccc7)ccc6C)ccc5cc4c2cc13)c1cc(-c2ccccc2)ccc1C]

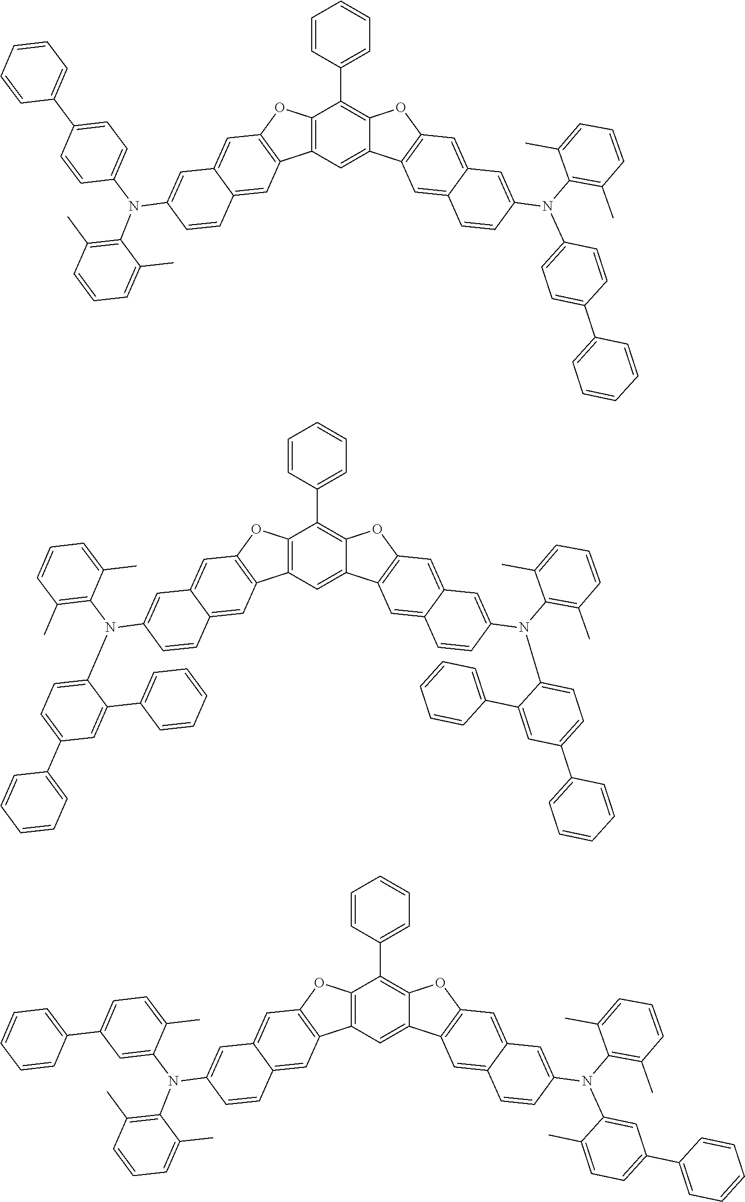 [SMILES: Cc1cccc(C)c1N(c1ccc(-c2ccccc2)cc1)c1ccc2cc3c(cc2c1)oc1c(-c2ccccc2)c2oc4cc5cc(N(c6ccc(-c7ccccc7)cc6)c6c(C)cccc6C)ccc5cc4c2cc13 ; Cc1cccc(C)c1N(c1ccc2cc3c(cc2c1)oc1c(-c2ccccc2)c2oc4cc5cc(N(c6ccc(-c7ccccc7)cc6-c6ccccc6)c6c(C)cccc6C)ccc5cc4c2cc13)c1ccc(-c2ccccc2)cc1-c1ccccc1 ; Cc1ccc(-c2ccccc2)cc1N(c1ccc2cc3c(cc2c1)oc1c(-c2ccccc2)c2oc4cc5cc(N(c6cc(-c7ccccc7)ccc6C)c6c(C)cccc6C)ccc5cc4c2cc13)c1c(C)cccc1C]

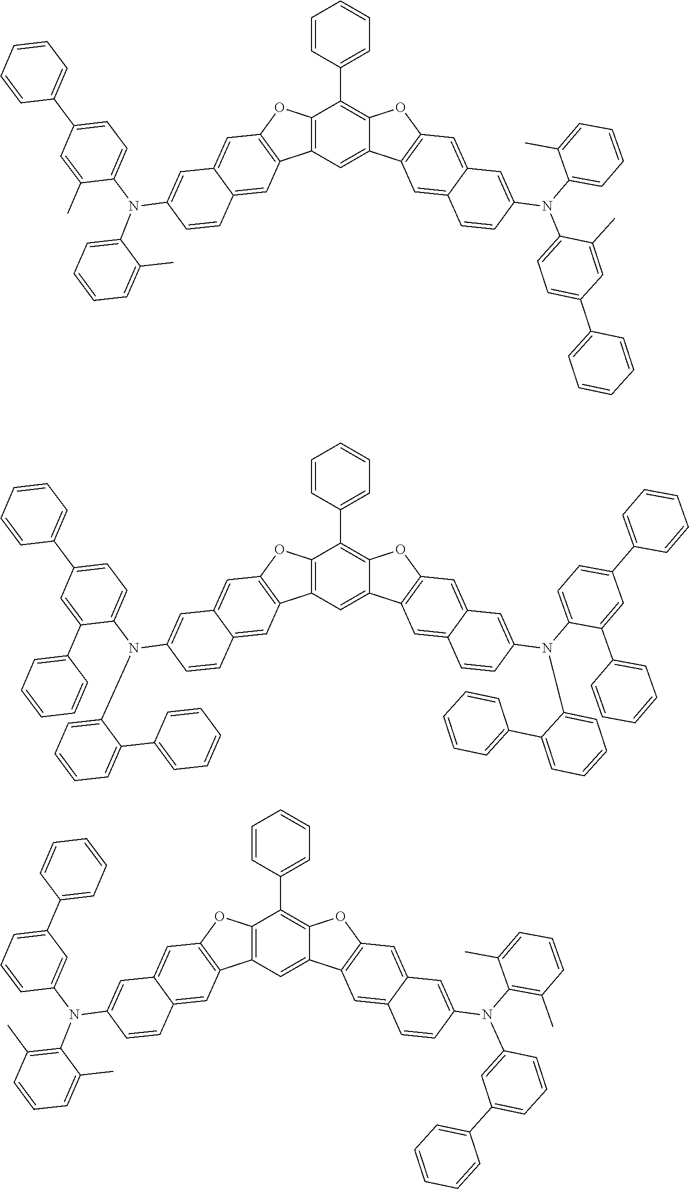 [SMILES: Cc1ccccc1N(c1ccc2cc3c(cc2c1)oc1c(-c2ccccc2)c2oc4cc5cc(N(c6ccccc6C)c6ccc(-c7ccccc7)cc6C)ccc5cc4c2cc13)c1ccc(-c2ccccc2)cc1C ; c1ccc(-c2ccc(N(c3ccc4cc5c(cc4c3)oc3c(-c4ccccc4)c4oc6cc7cc(N(c8ccccc8-c8ccccc8)c8ccc(-c9ccccc9)cc8-c8ccccc8)ccc7cc6c4cc35)c3ccccc3-c3ccccc3)c(-c3ccccc3)c2)cc1 ; Cc1cccc(C)c1N(c1cccc(-c2ccccc2)c1)c1ccc2cc3c(cc2c1)oc1c(-c2ccccc2)c2oc4cc5cc(N(c6cccc(-c7ccccc7)c6)c6c(C)cccc6C)ccc5cc4c2cc13]

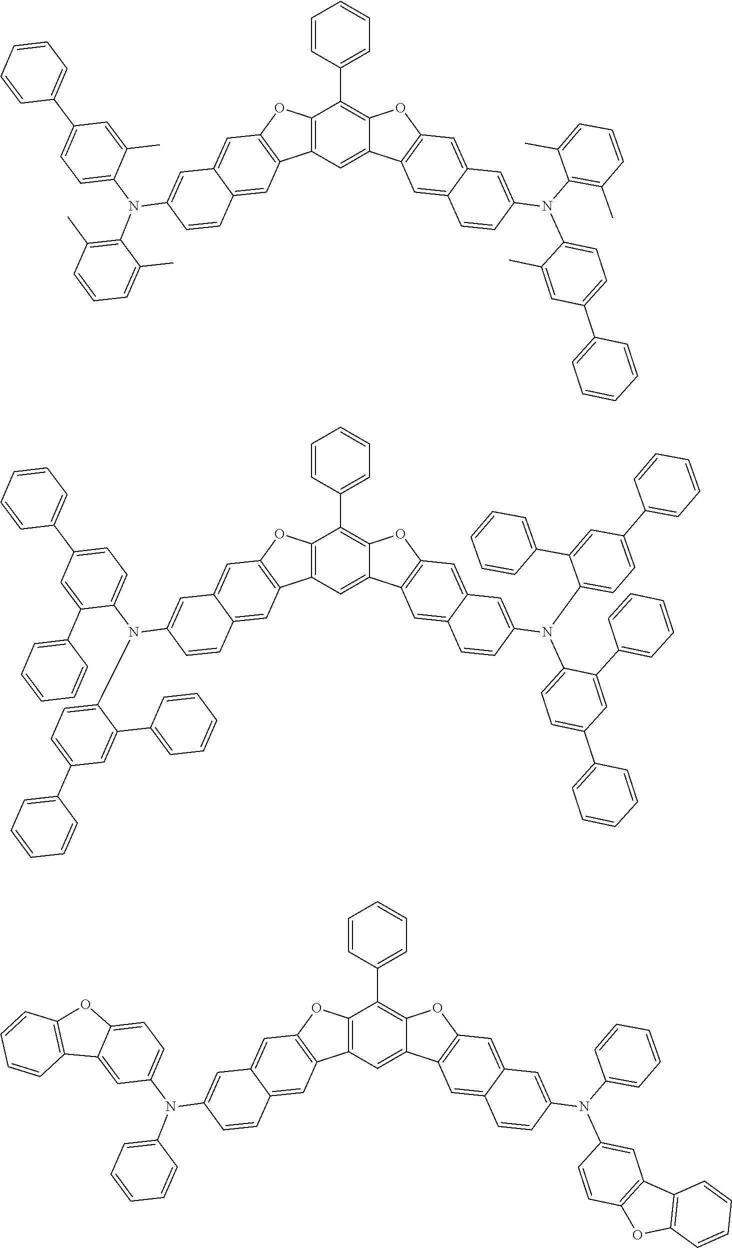 [SMILES: Cc1cc(-c2ccccc2)ccc1N(c1ccc2cc3c(cc2c1)oc1c(-c2ccccc2)c2oc4cc5cc(N(c6ccc(-c7ccccc7)cc6C)c6c(C)cccc6C)ccc5cc4c2cc13)c1c(C)cccc1C ; c1ccc(-c2ccc(N(c3ccc4cc5c(cc4c3)oc3c(-c4ccccc4)c4oc6cc7cc(N(c8ccc(-c9ccccc9)cc8-c8ccccc8)c8ccc(-c9ccccc9)cc8-c8ccccc8)ccc7cc6c4cc35)c3ccc(-c4ccccc4)cc3-c3ccccc3)c(-c3ccccc3)c2)cc1 ; c1ccc(-c2c3oc4cc5cc(N(c6ccccc6)c6ccc7oc8ccccc8c7c6)ccc5cc4c3cc3c2oc2cc4cc(N(c5ccccc5)c5ccc6oc7ccccc7c6c5)ccc4cc23)cc1]

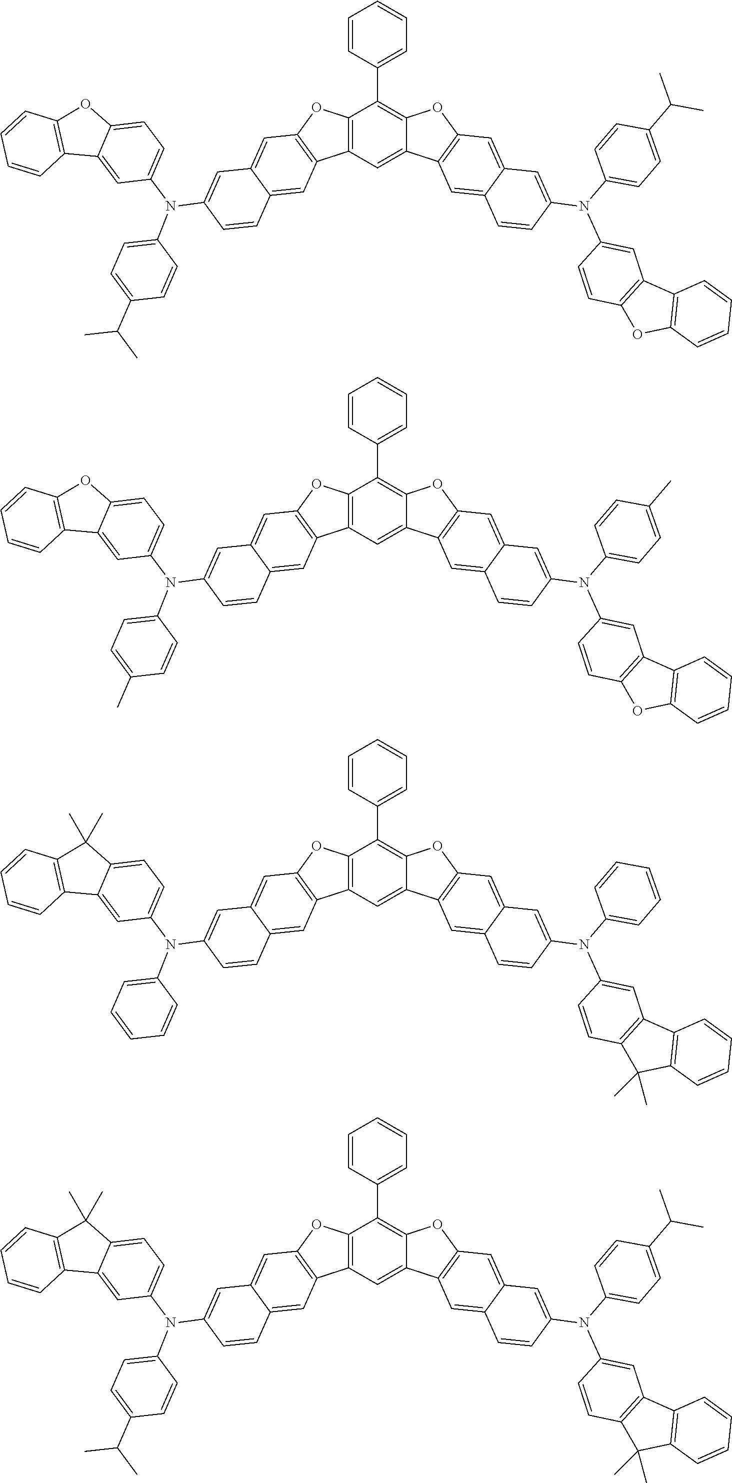

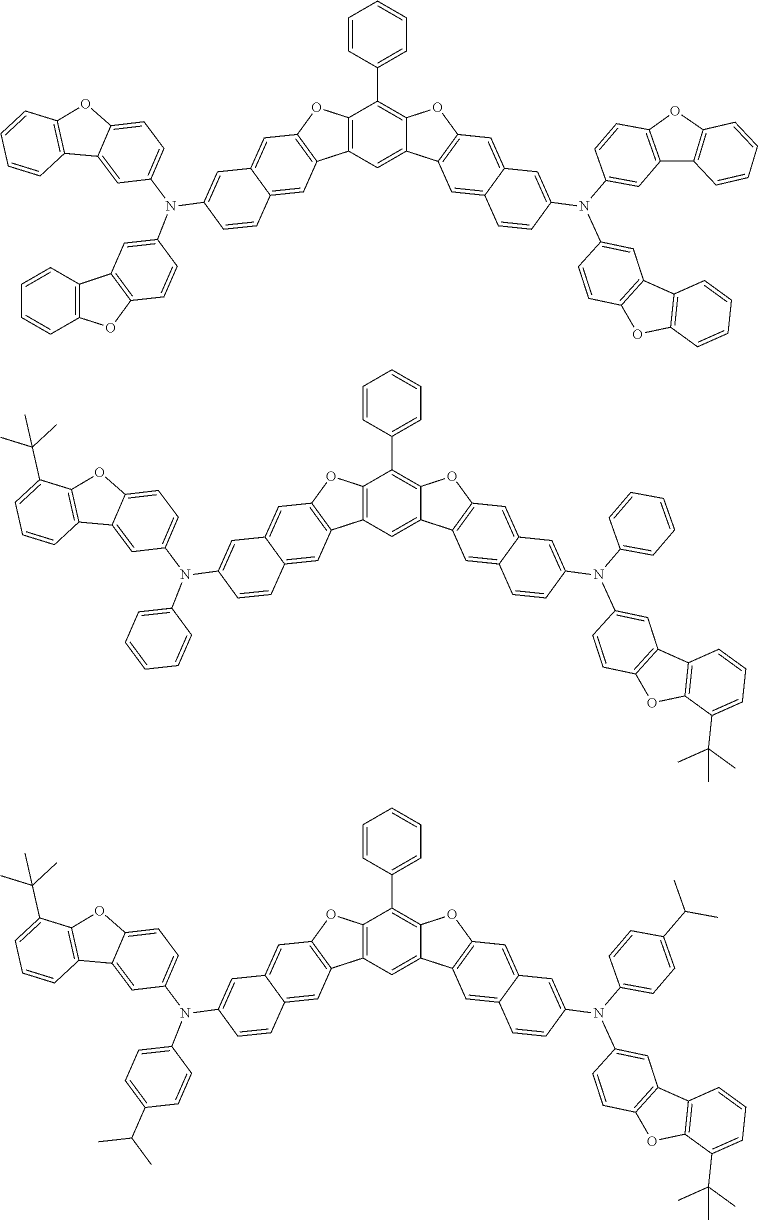 [SMILES: c1ccc(-c2c3oc4cc5cc(N(c6ccc7oc8ccccc8c7c6)c6ccc7oc8ccccc8c7c6)ccc5cc4c3cc3c2oc2cc4cc(N(c5ccc6oc7ccccc7c6c5)c5ccc6oc7ccccc7c6c5)ccc4cc23)cc1 ; CC(C)(C)c1cccc2c1oc1ccc(N(c3ccccc3)c3ccc4cc5c(cc4c3)oc3c(-c4ccccc4)c4oc6cc7cc(N(c8ccccc8)c8ccc9oc%10c(C(C)(C)C)cccc%10c9c8)ccc7cc6c4cc35)cc12 ; CC(C)c1ccc(N(c2ccc3cc4c(cc3c2)oc2c(-c3ccccc3)c3oc5cc6cc(N(c7ccc(C(C)C)cc7)c7ccc8oc9c(C(C)(C)C)cccc9c8c7)ccc6cc5c3cc24)c2ccc3oc4c(C(C)(C)C)cccc4c3c2)cc1]

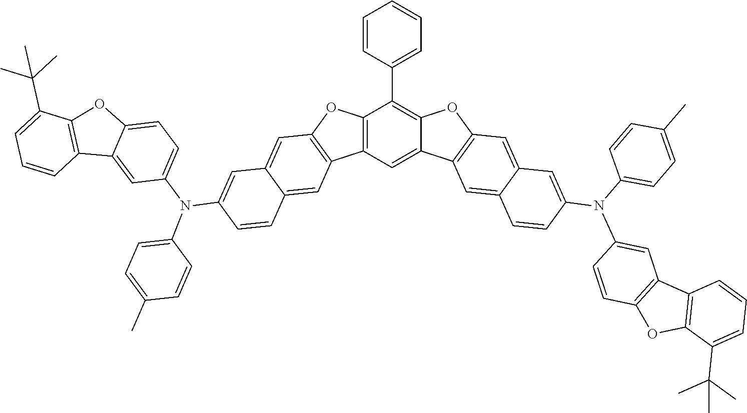 [SMILES: Cc1ccc(N(c2ccc3cc4c(cc3c2)oc2c(-c3ccccc3)c3oc5cc6cc(N(c7ccc(C)cc7)c7ccc8oc9c(C(C)(C)C)cccc9c8c7)ccc6cc5c3cc24)c2ccc3oc4c(C(C)(C)C)cccc4c3c2)cc1]

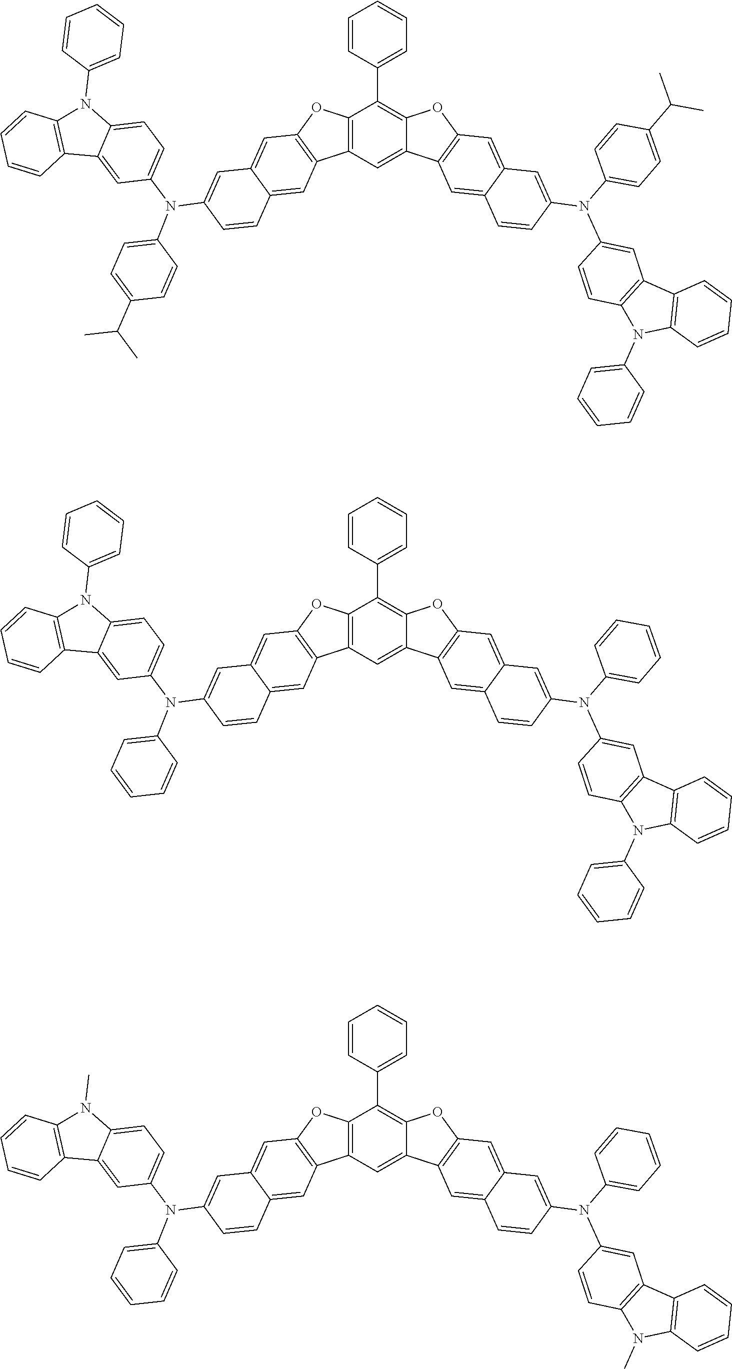 [SMILES: CC(C)c1ccc(N(c2ccc3cc4c(cc3c2)oc2c(-c3ccccc3)c3oc5cc6cc(N(c7ccc(C(C)C)cc7)c7ccc8c(c7)c7ccccc7n8-c7ccccc7)ccc6cc5c3cc24)c2ccc3c(c2)c2ccccc2n3-c2ccccc2)cc1 ; c1ccc(-c2c3oc4cc5cc(N(c6ccccc6)c6ccc7c(c6)c6ccccc6n7-c6ccccc6)ccc5cc4c3cc3c2oc2cc4cc(N(c5ccccc5)c5ccc6c(c5)c5ccccc5n6-c5ccccc5)ccc4cc23)cc1 ; Cn1c2ccccc2c2cc(N(c3ccccc3)c3ccc4cc5c(cc4c3)oc3c(-c4ccccc4)c4oc6cc7cc(N(c8ccccc8)c8ccc9c(c8)c8ccccc8n9C)ccc7cc6c4cc35)ccc21]

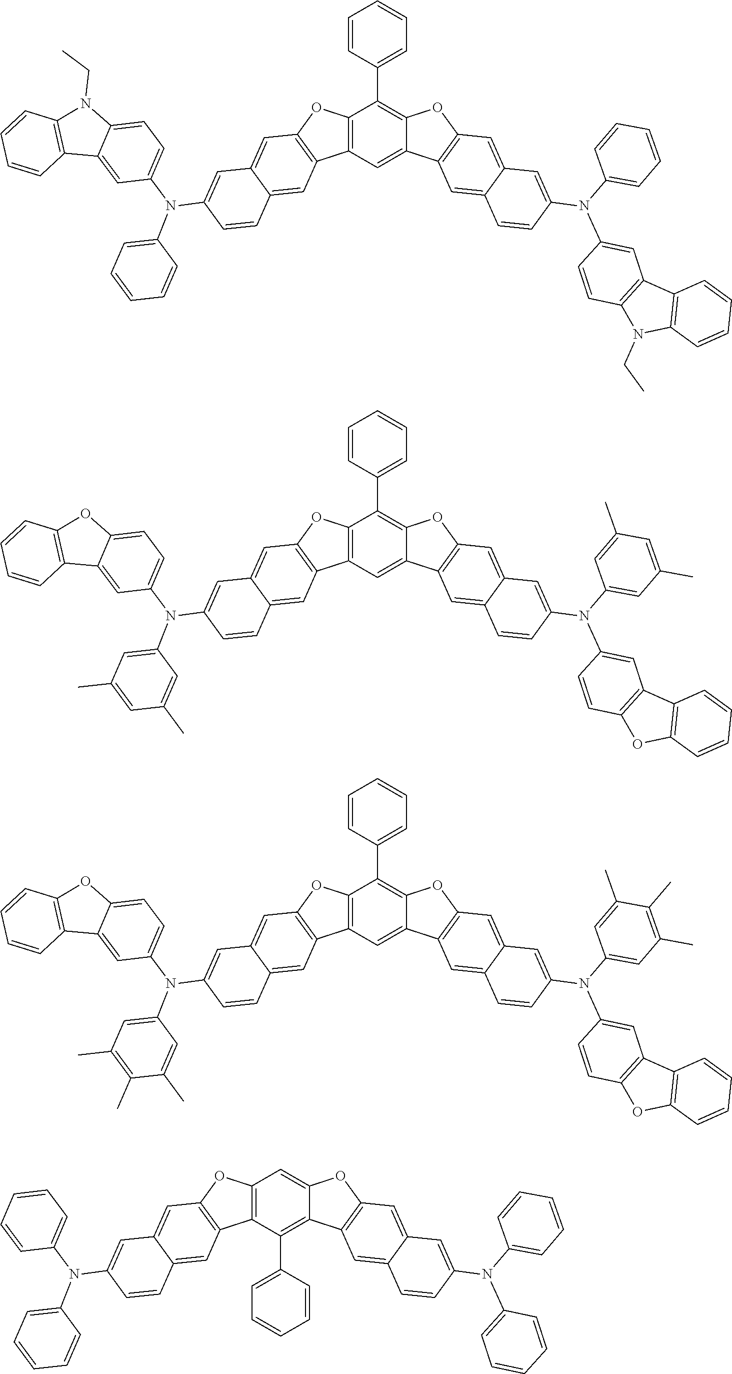 [SMILES: CCn1c2ccccc2c2cc(N(c3ccccc3)c3ccc4cc5c(cc4c3)oc3c(-c4ccccc4)c4oc6cc7cc(N(c8ccccc8)c8ccc9c(c8)c8ccccc8n9CC)ccc7cc6c4cc35)ccc21 ; Cc1cc(C)cc(N(c2ccc3cc4c(cc3c2)oc2c(-c3ccccc3)c3oc5cc6cc(N(c7cc(C)cc(C)c7)c7ccc8oc9ccccc9c8c7)ccc6cc5c3cc24)c2ccc3oc4ccccc4c3c2)c1 ; Cc1cc(N(c2ccc3cc4c(cc3c2)oc2c(-c3ccccc3)c3oc5cc6cc(N(c7cc(C)c(C)c(C)c7)c7ccc8oc9ccccc9c8c7)ccc6cc5c3cc24)c2ccc3oc4ccccc4c3c2)cc(C)c1C ; c1ccc(-c2c3c(cc4oc5cc6cc(N(c7ccccc7)c7ccccc7)ccc6cc5c24)oc2cc4cc(N(c5ccccc5)c5ccccc5)ccc4cc23)cc1]

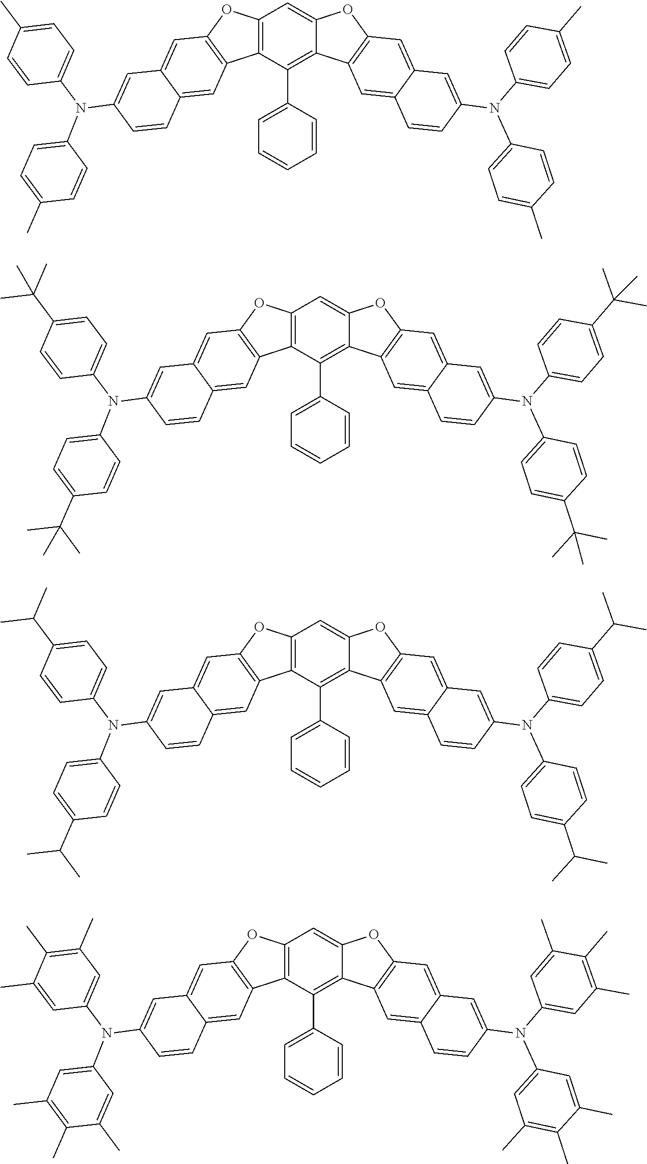

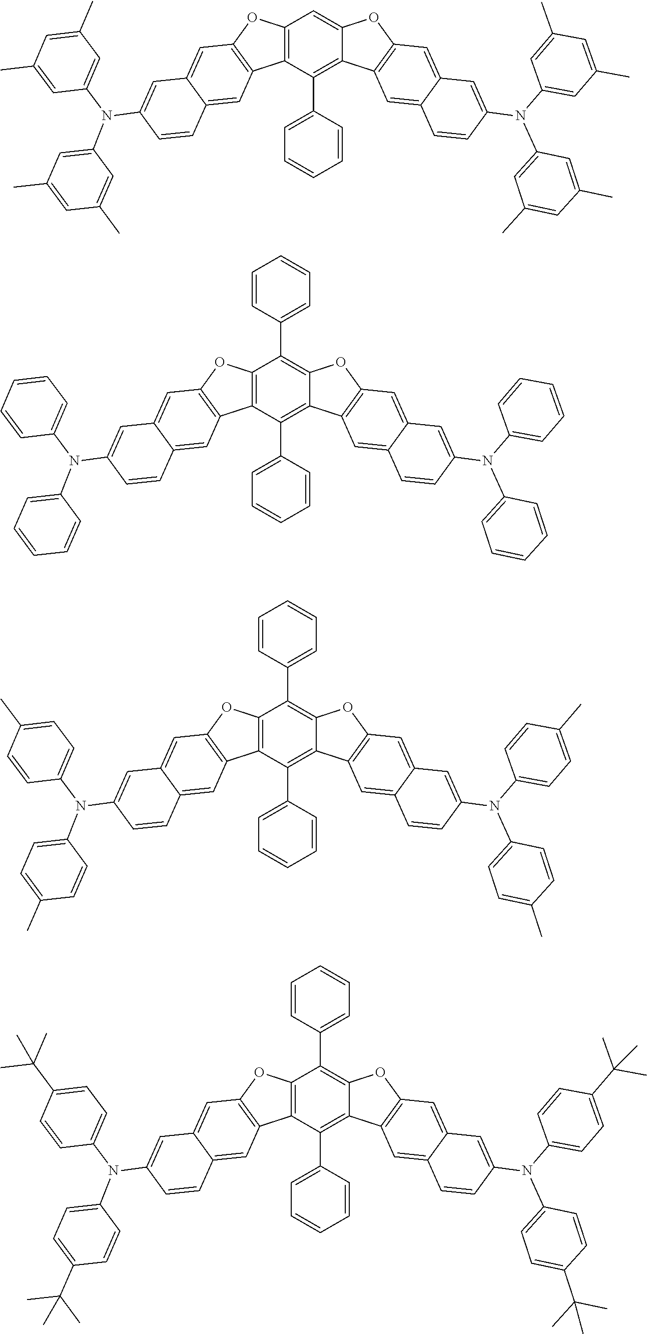

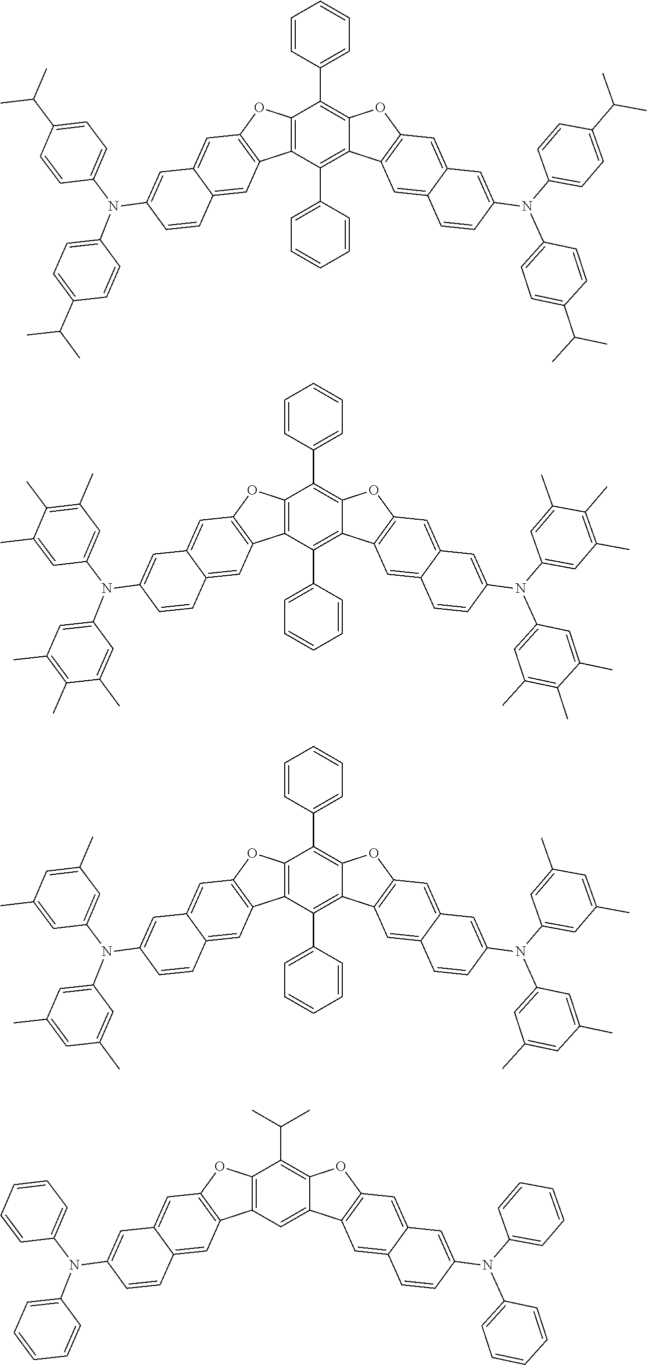

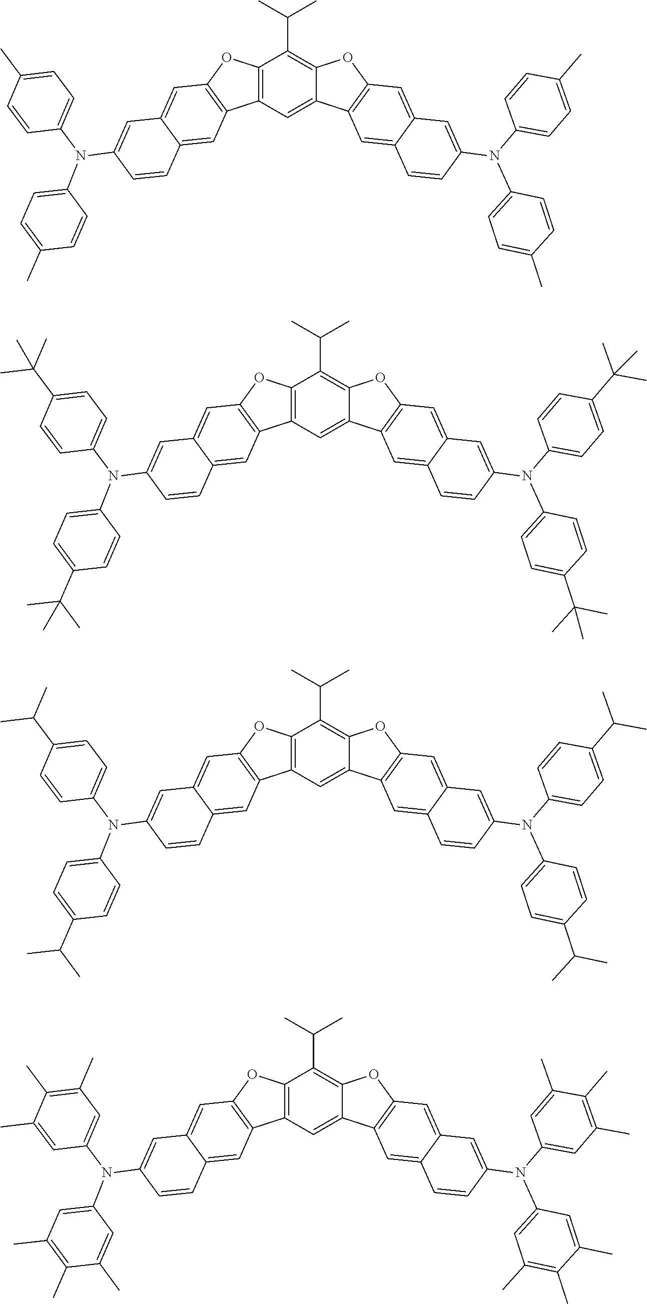

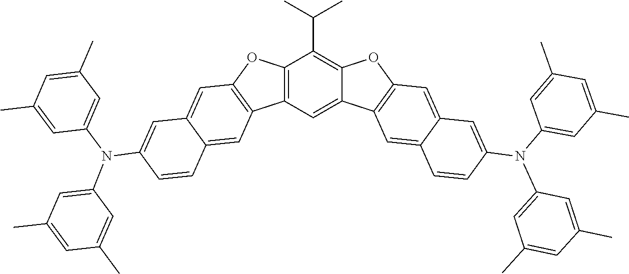

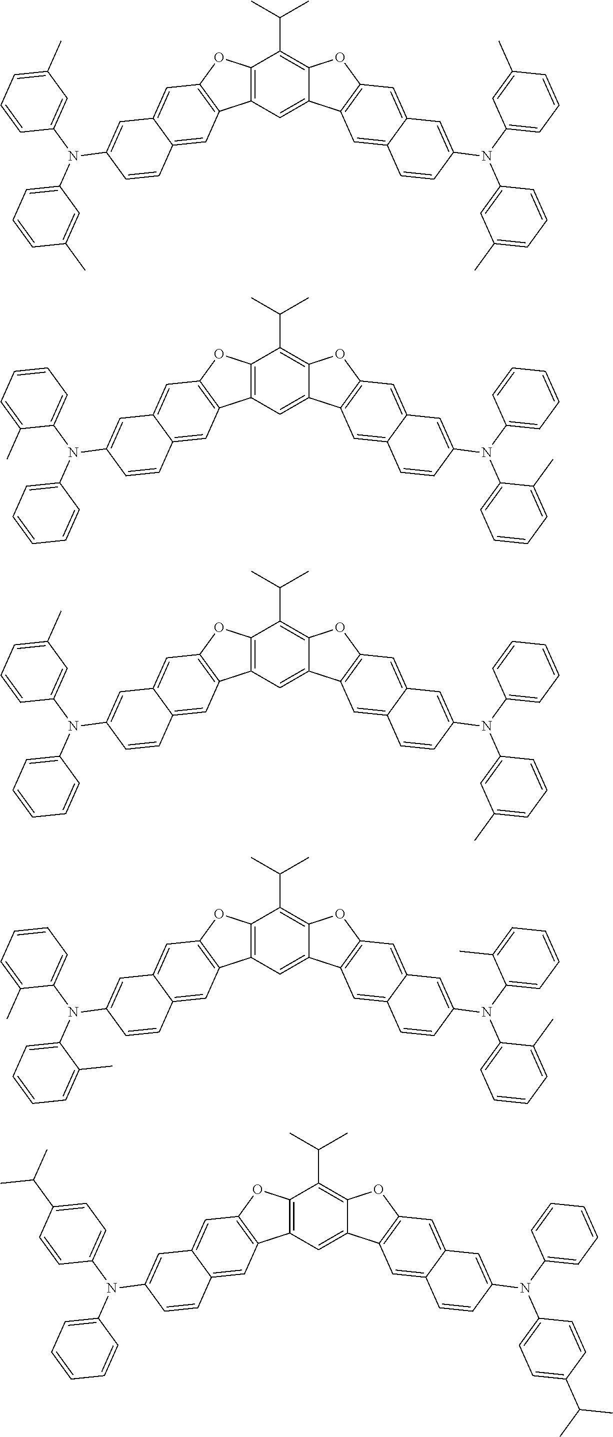

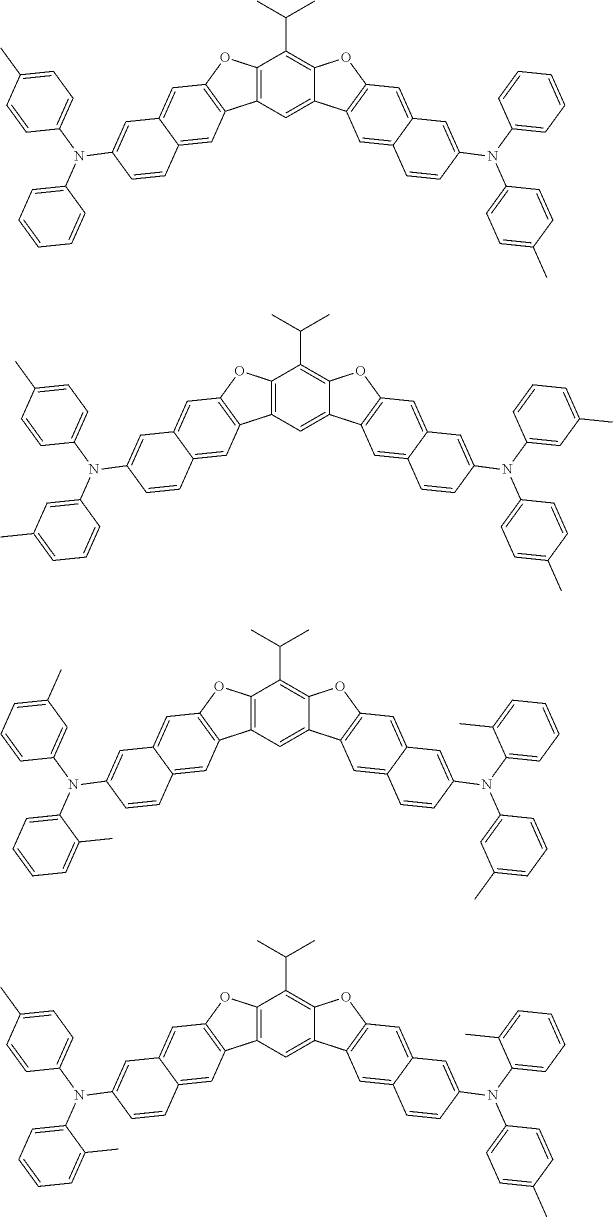

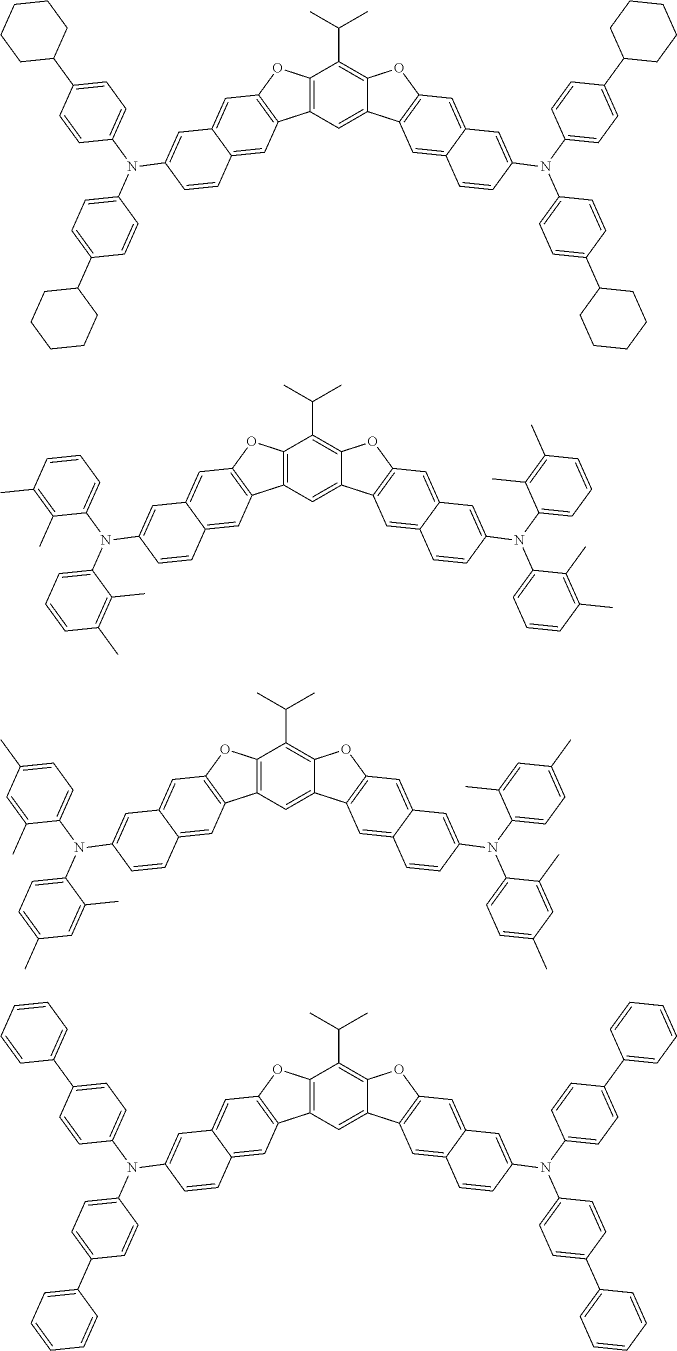

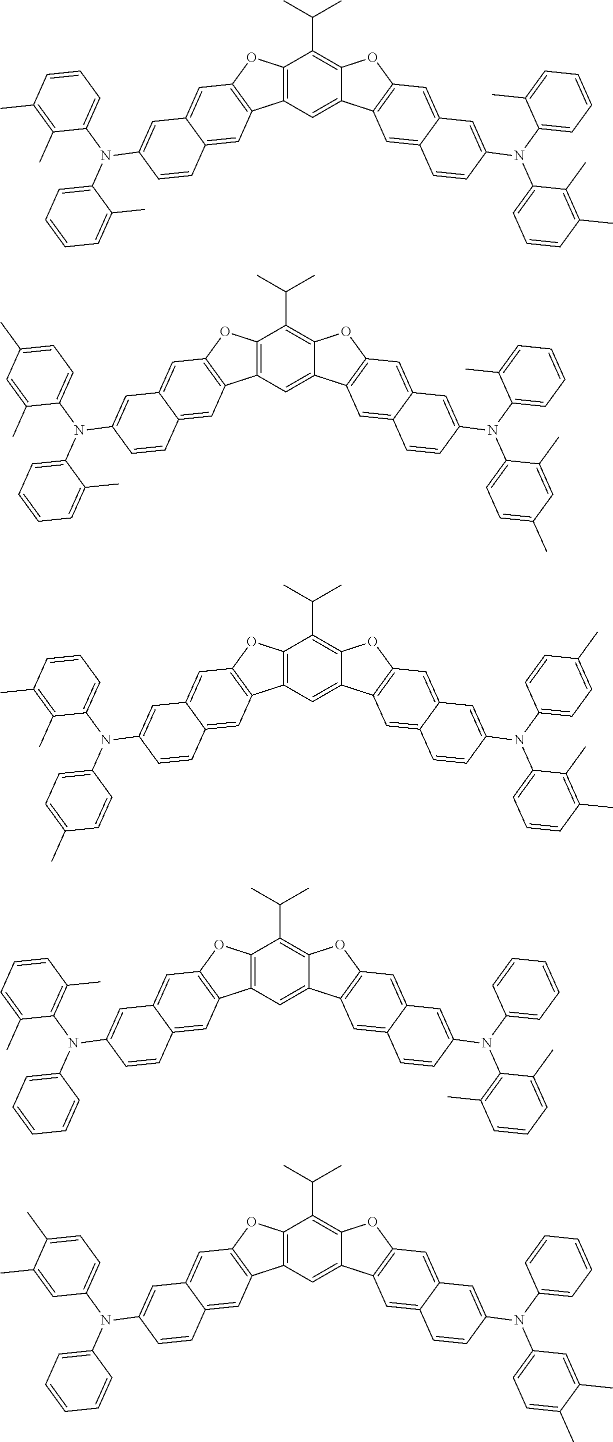

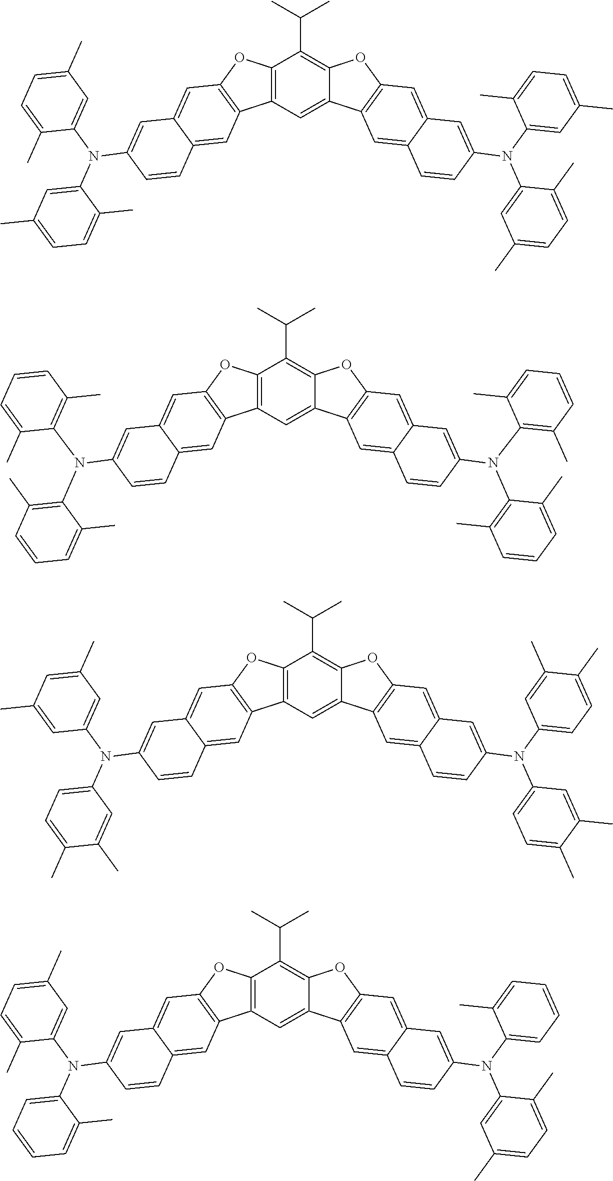

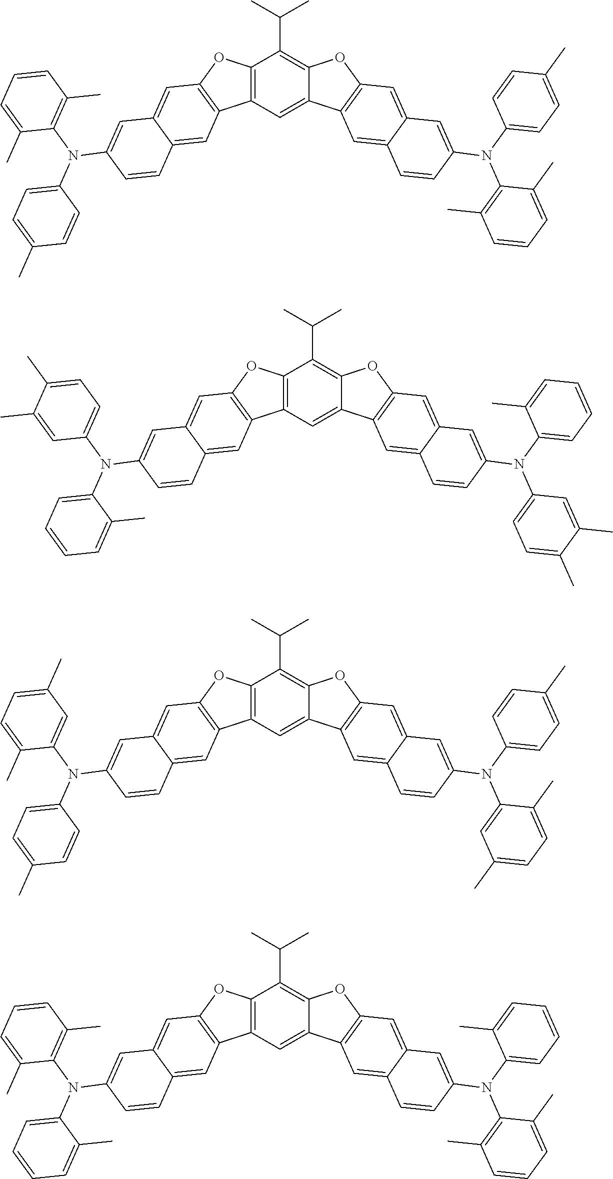

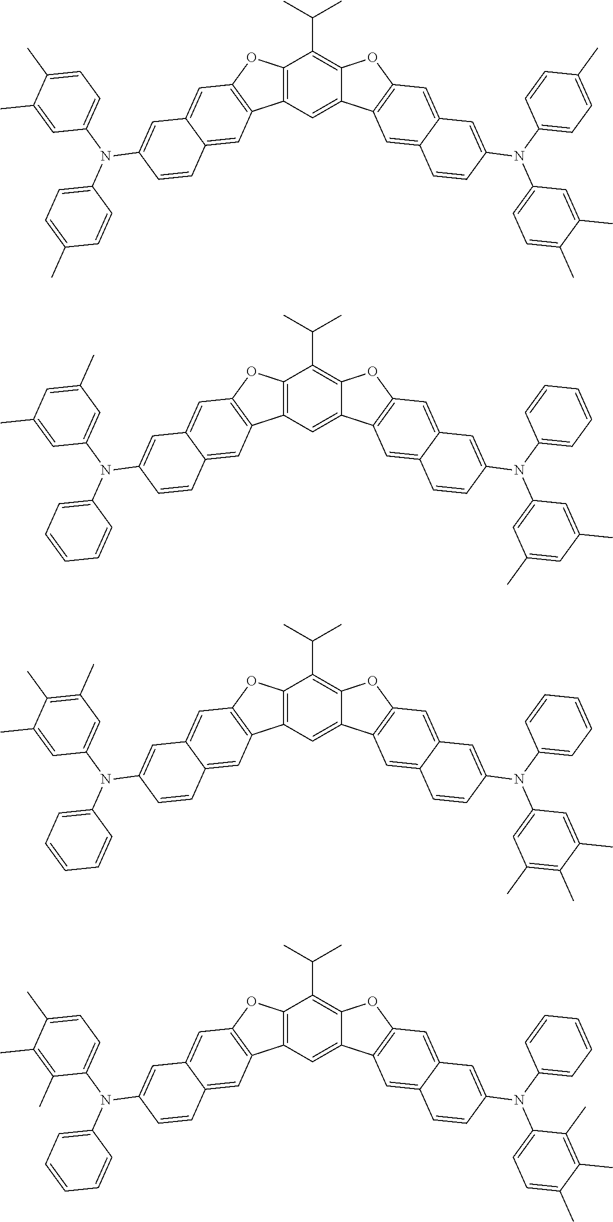

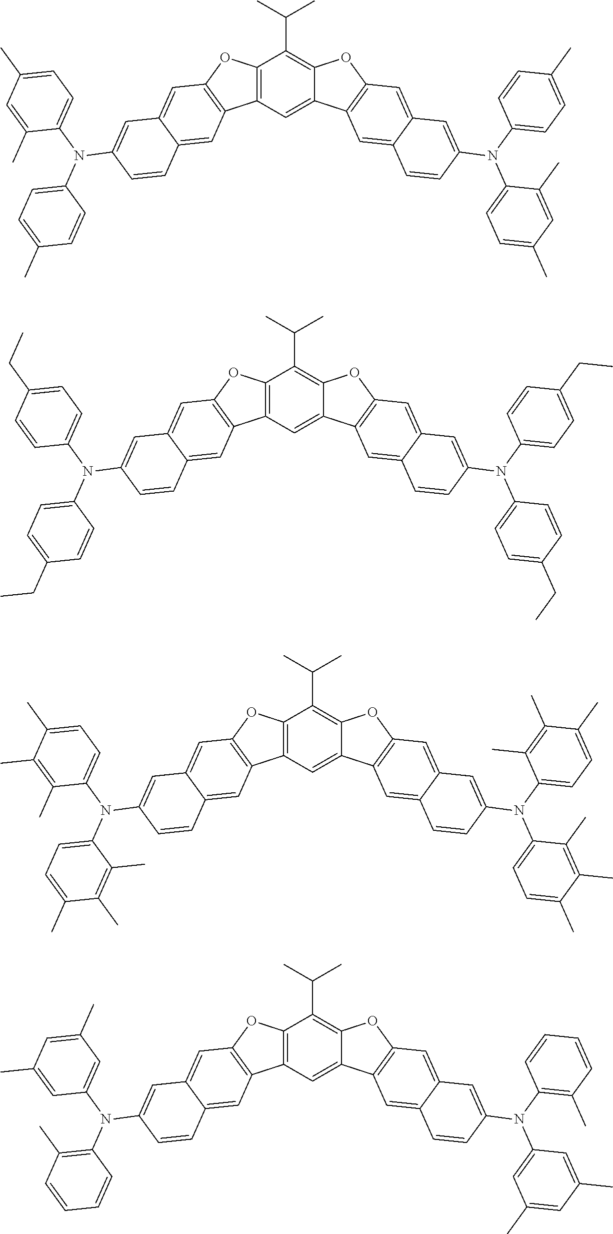

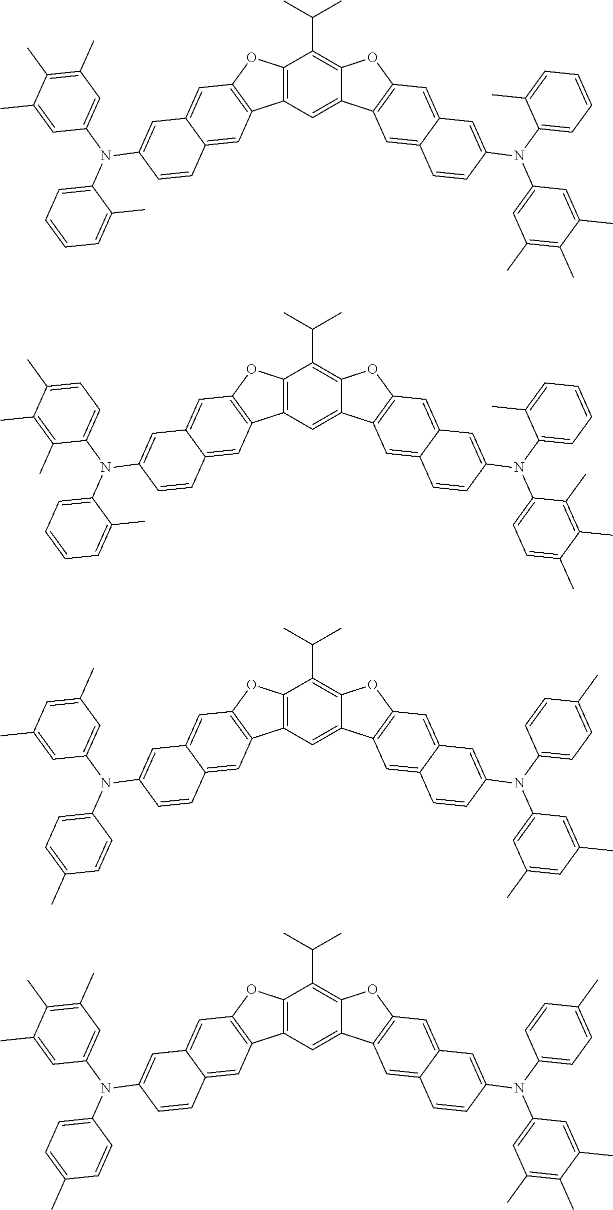

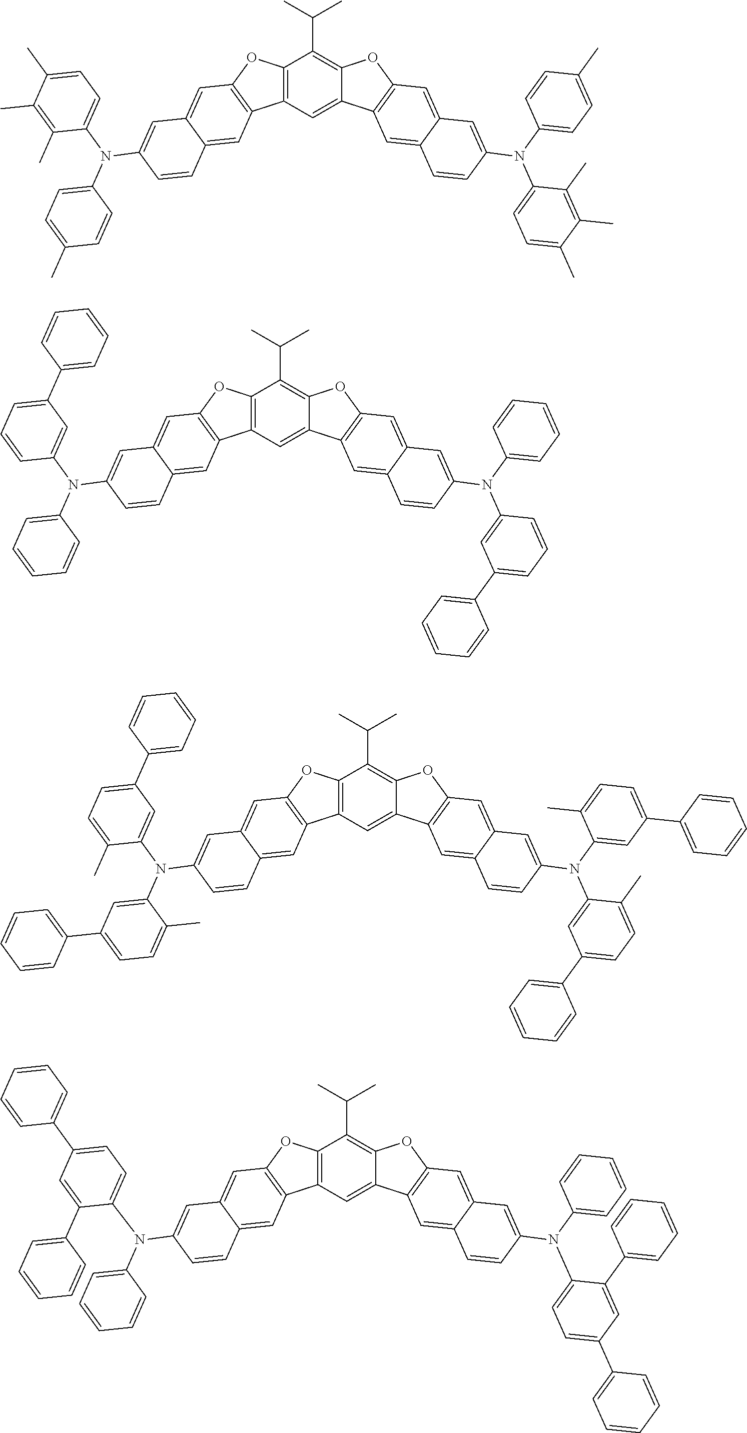

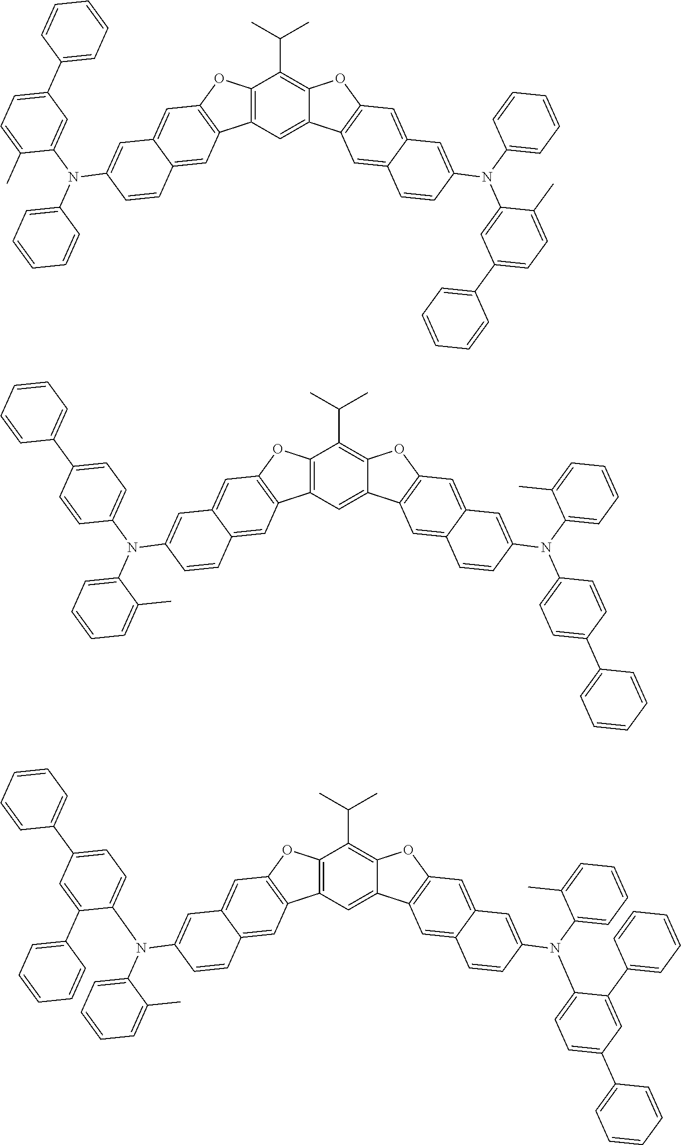 [SMILES: Cc1ccc(-c2ccccc2)cc1N(c1ccccc1)c1ccc2cc3c(cc2c1)oc1c(C(C)C)c2oc4cc5cc(N(c6ccccc6)c6cc(-c7ccccc7)ccc6C)ccc5cc4c2cc13 ; Cc1ccccc1N(c1ccc(-c2ccccc2)cc1)c1ccc2cc3c(cc2c1)oc1c(C(C)C)c2oc4cc5cc(N(c6ccc(-c7ccccc7)cc6)c6ccccc6C)ccc5cc4c2cc13 ; Cc1ccccc1N(c1ccc2cc3c(cc2c1)oc1c(C(C)C)c2oc4cc5cc(N(c6ccccc6C)c6ccc(-c7ccccc7)cc6-c6ccccc6)ccc5cc4c2cc13)c1ccc(-c2ccccc2)cc1-c1ccccc1]

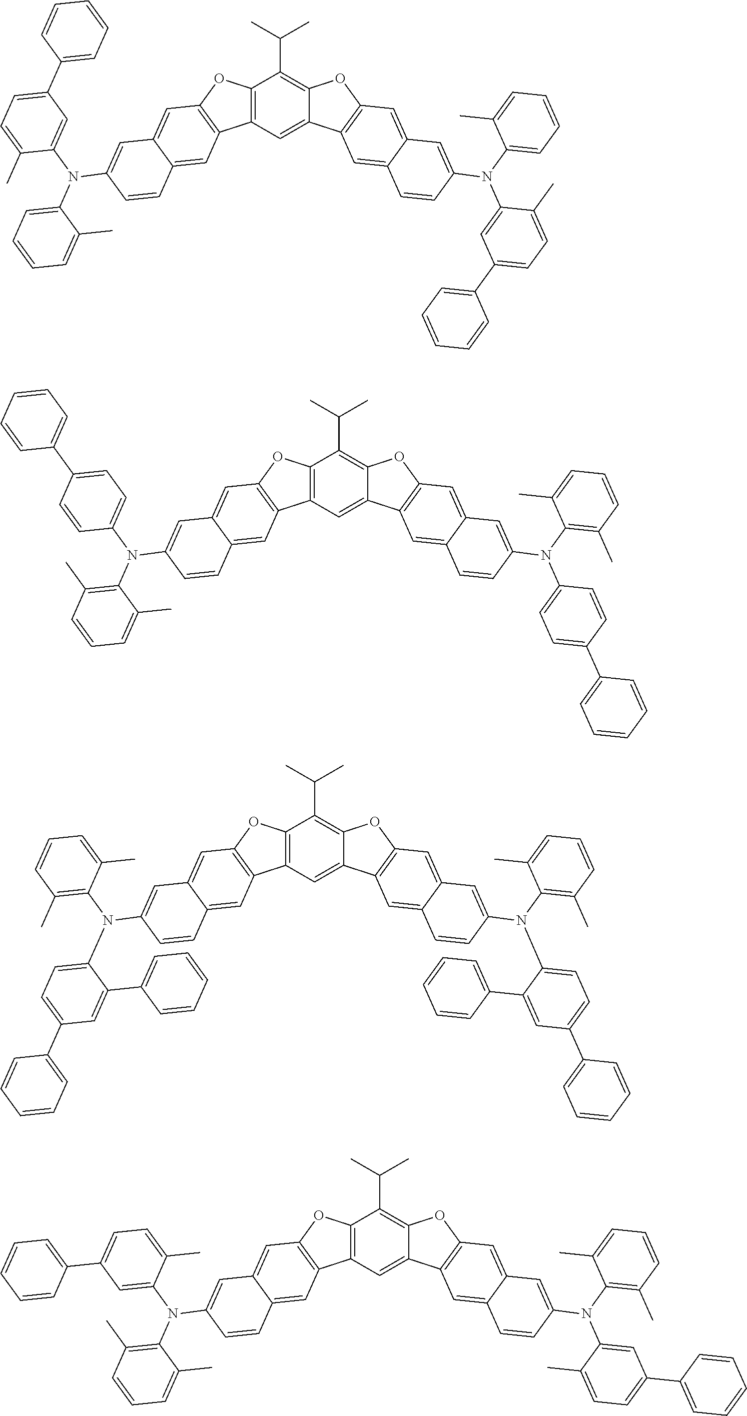

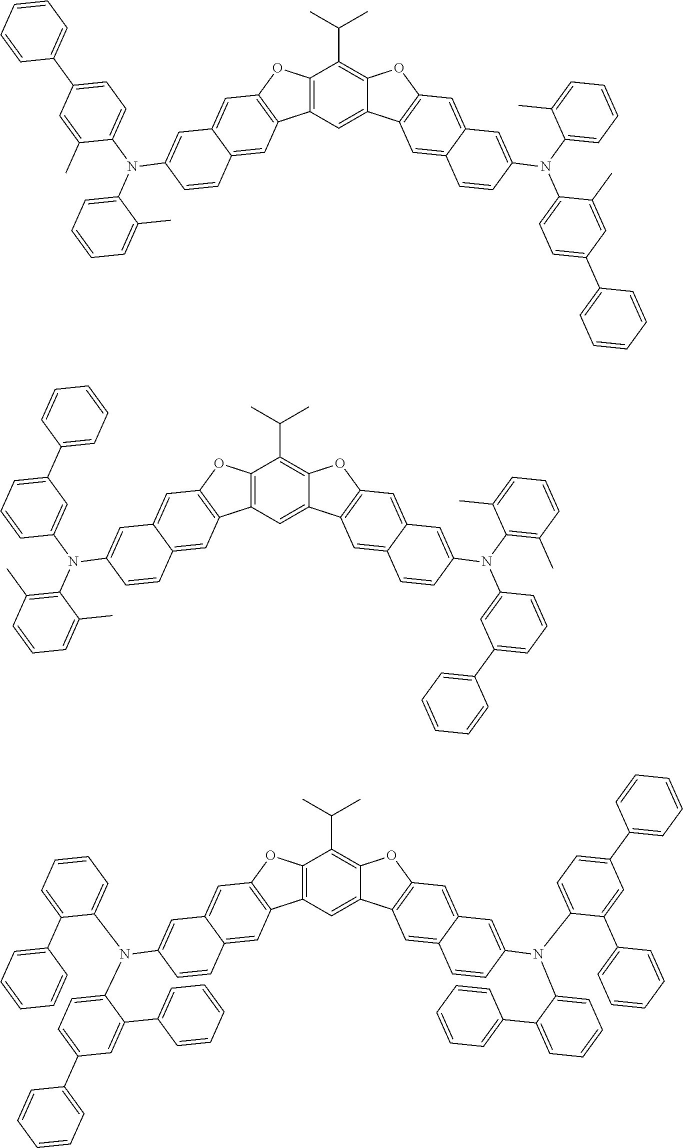 [SMILES: Cc1ccccc1N(c1ccc2cc3c(cc2c1)oc1c(C(C)C)c2oc4cc5cc(N(c6ccccc6C)c6ccc(-c7ccccc7)cc6C)ccc5cc4c2cc13)c1ccc(-c2ccccc2)cc1C ; Cc1cccc(C)c1N(c1cccc(-c2ccccc2)c1)c1ccc2cc3c(cc2c1)oc1c(C(C)C)c2oc4cc5cc(N(c6cccc(-c7ccccc7)c6)c6c(C)cccc6C)ccc5cc4c2cc13 ; CC(C)c1c2oc3cc4cc(N(c5ccccc5-c5ccccc5)c5ccc(-c6ccccc6)cc5-c5ccccc5)ccc4cc3c2cc2c1oc1cc3cc(N(c4ccccc4-c4ccccc4)c4ccc(-c5ccccc5)cc4-c4ccccc4)ccc3cc12]

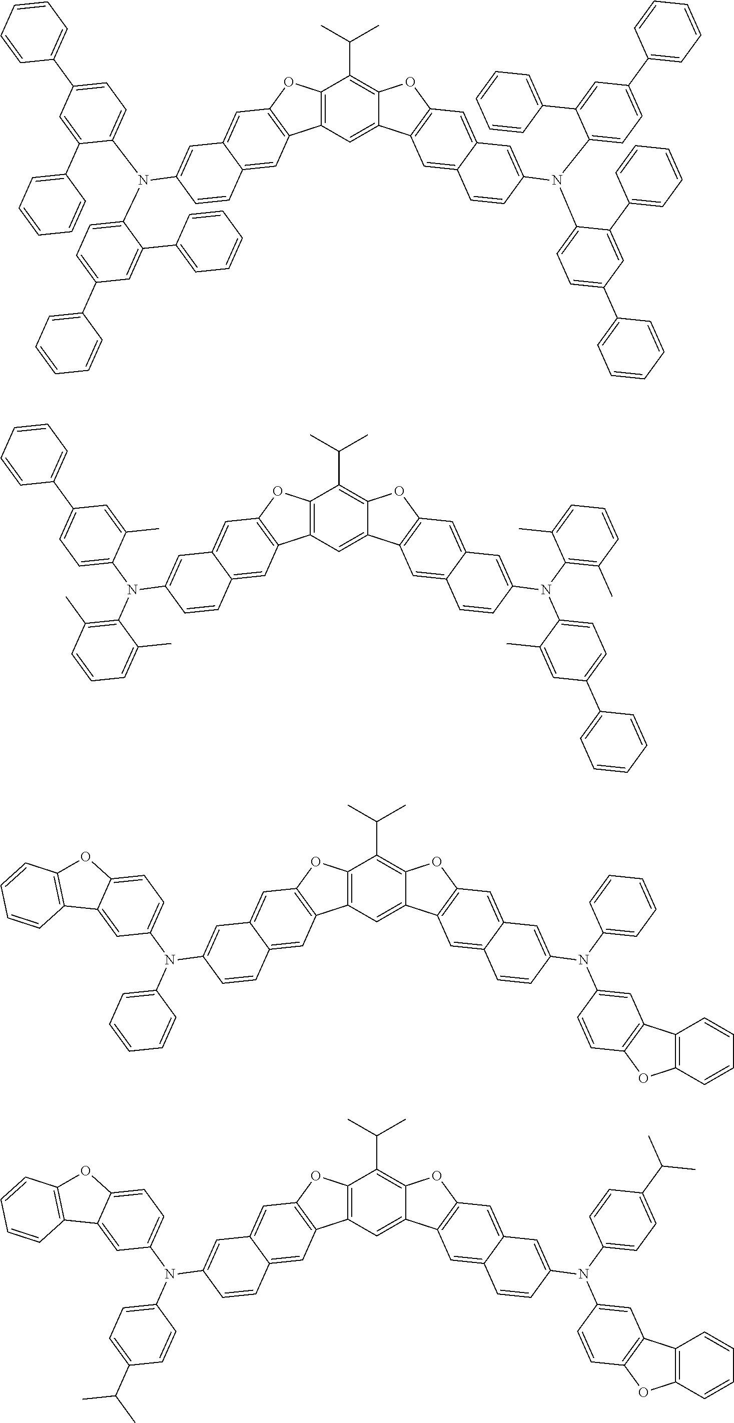

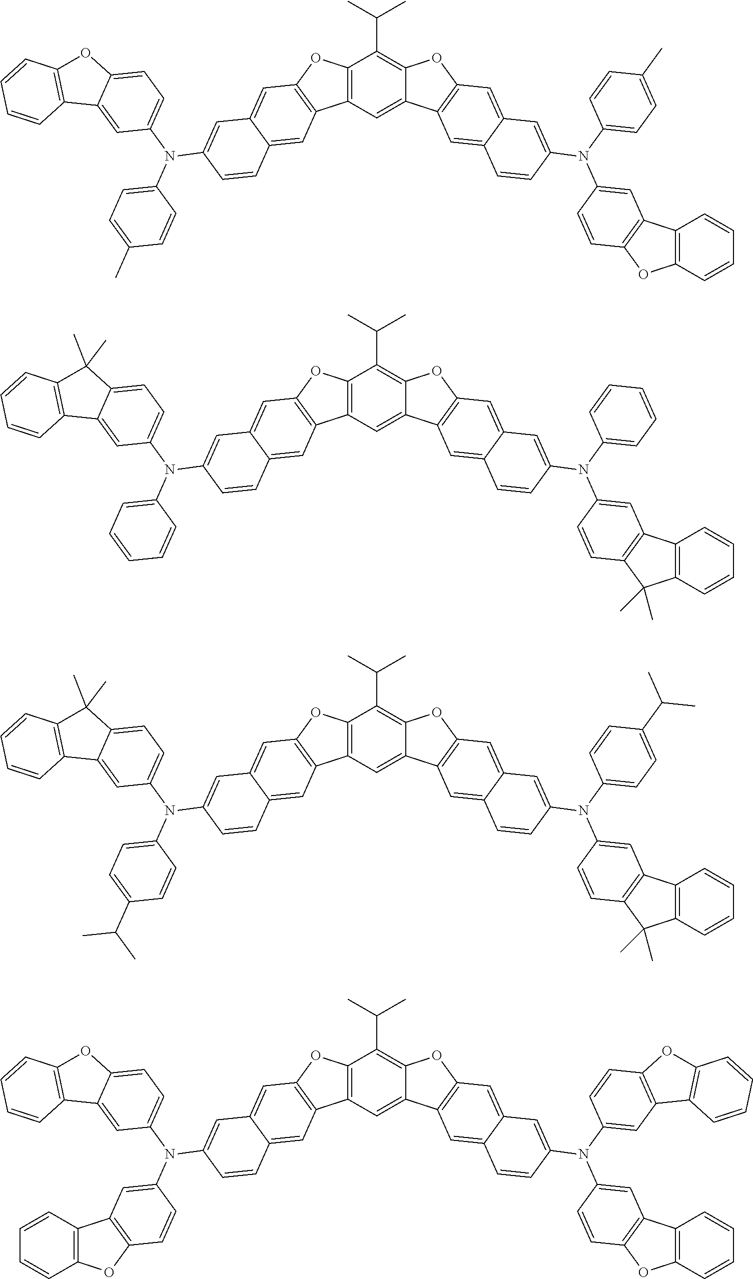

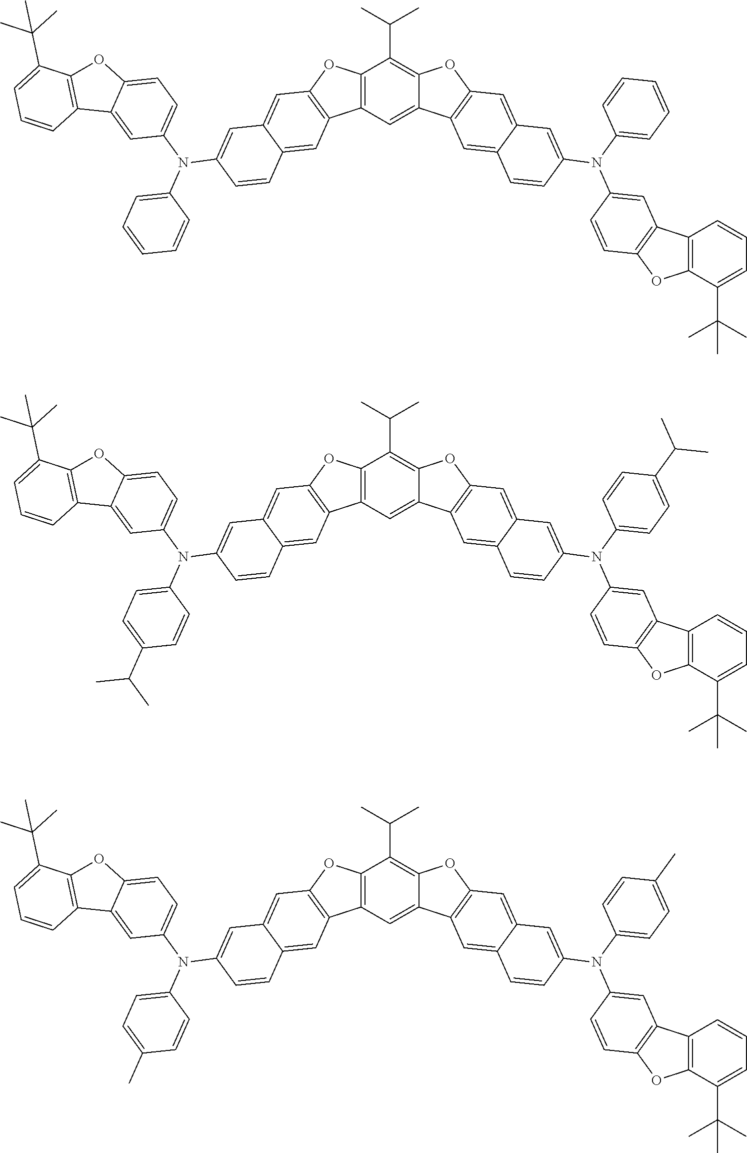

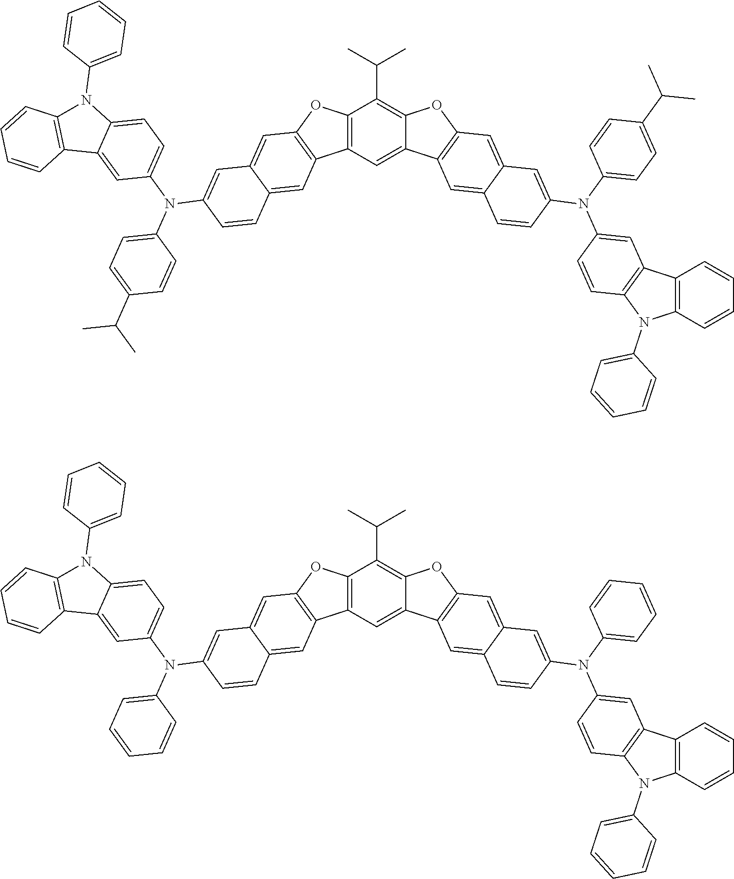 [SMILES: CC(C)c1ccc(N(c2ccc3cc4c(cc3c2)oc2c(C(C)C)c3oc5cc6cc(N(c7ccc(C(C)C)cc7)c7ccc8c(c7)c7ccccc7n8-c7ccccc7)ccc6cc5c3cc24)c2ccc3c(c2)c2ccccc2n3-c2ccccc2)cc1 ; CC(C)c1c2oc3cc4cc(N(c5ccccc5)c5ccc6c(c5)c5ccccc5n6-c5ccccc5)ccc4cc3c2cc2c1oc1cc3cc(N(c4ccccc4)c4ccc5c(c4)c4ccccc4n5-c4ccccc4)ccc3cc12]

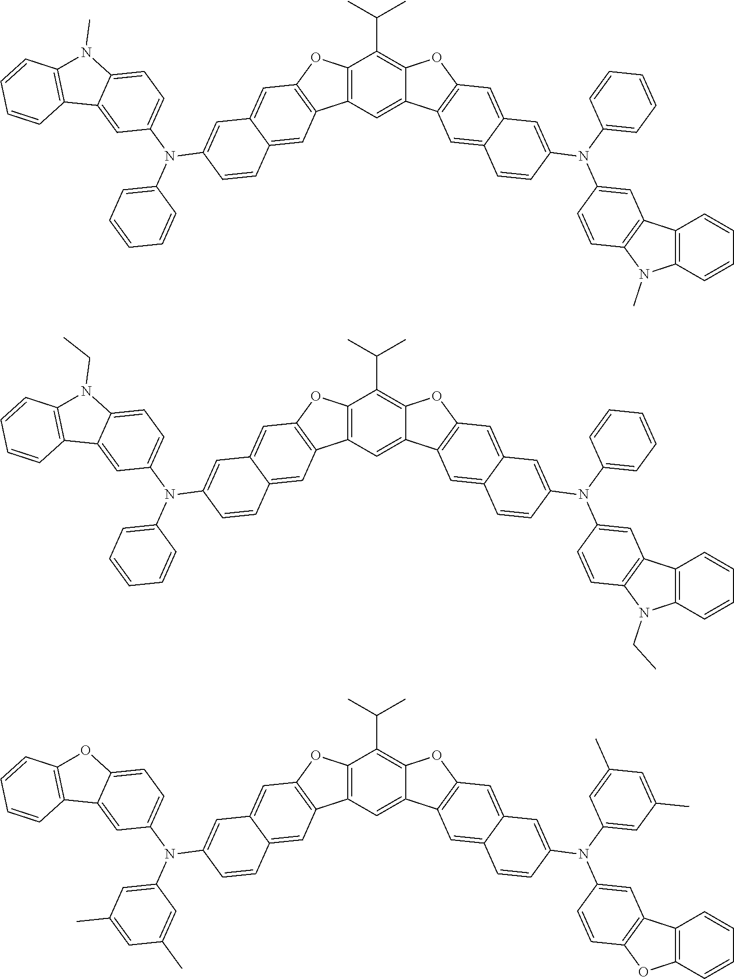 [SMILES: CC(C)c1c2oc3cc4cc(N(c5ccccc5)c5ccc6c(c5)c5ccccc5n6C)ccc4cc3c2cc2c1oc1cc3cc(N(c4ccccc4)c4ccc5c(c4)c4ccccc4n5C)ccc3cc12 ; CCn1c2ccccc2c2cc(N(c3ccccc3)c3ccc4cc5c(cc4c3)oc3c(C(C)C)c4oc6cc7cc(N(c8ccccc8)c8ccc9c(c8)c8ccccc8n9CC)ccc7cc6c4cc35)ccc21 ; Cc1cc(C)cc(N(c2ccc3cc4c(cc3c2)oc2c(C(C)C)c3oc5cc6cc(N(c7cc(C)cc(C)c7)c7ccc8oc9ccccc9c8c7)ccc6cc5c3cc24)c2ccc3oc4ccccc4c3c2)c1]

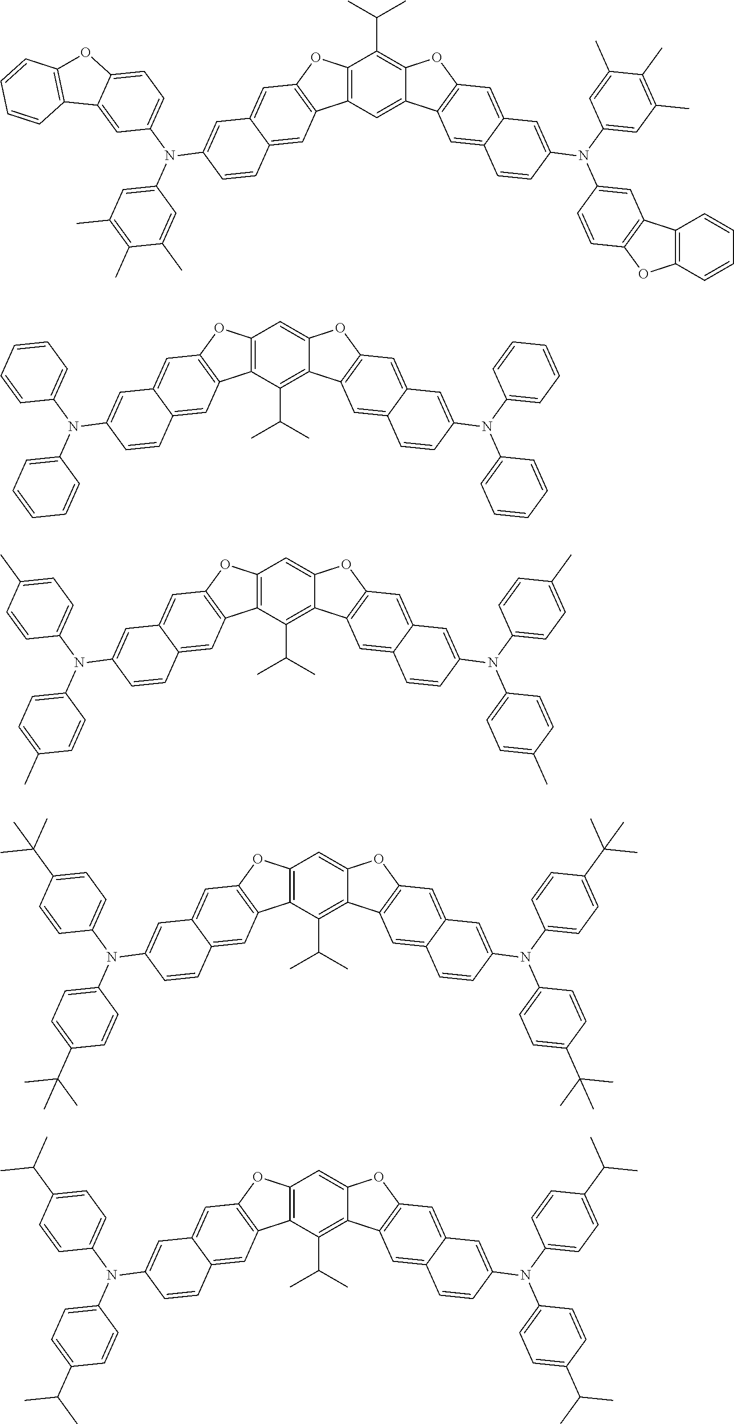

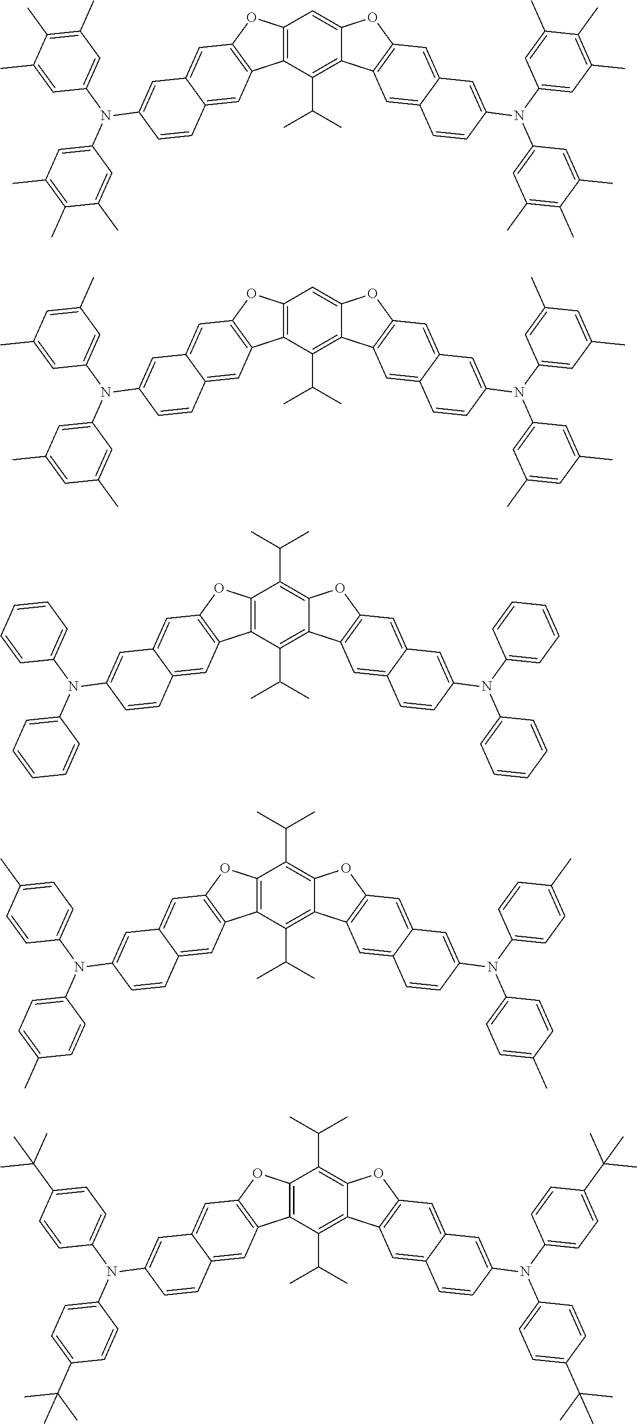

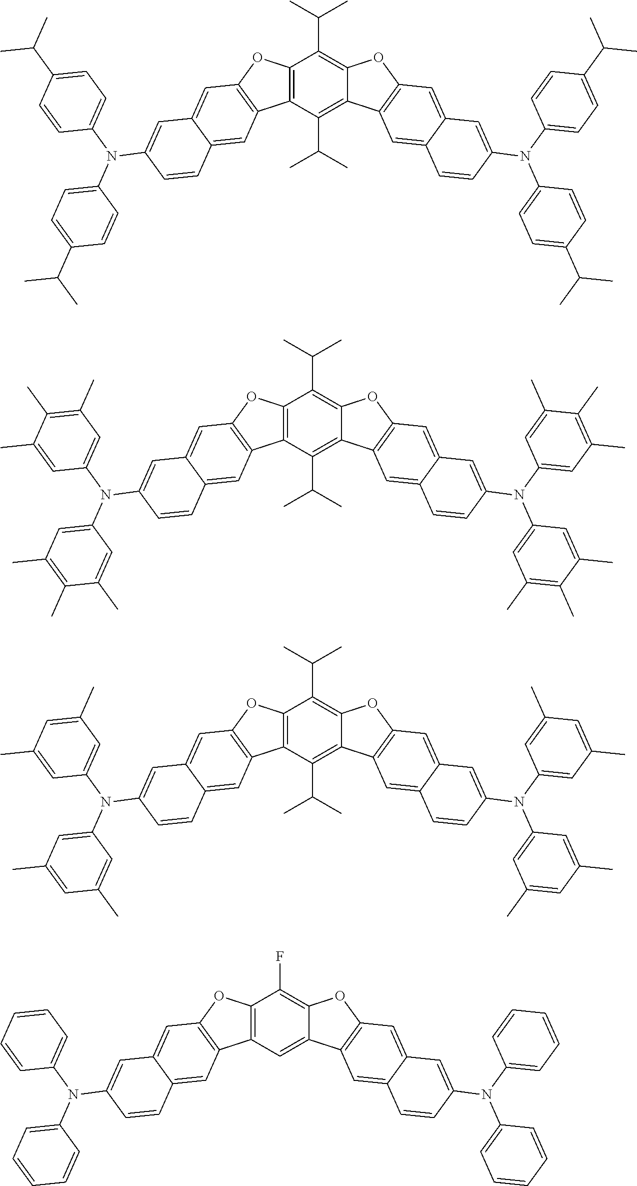

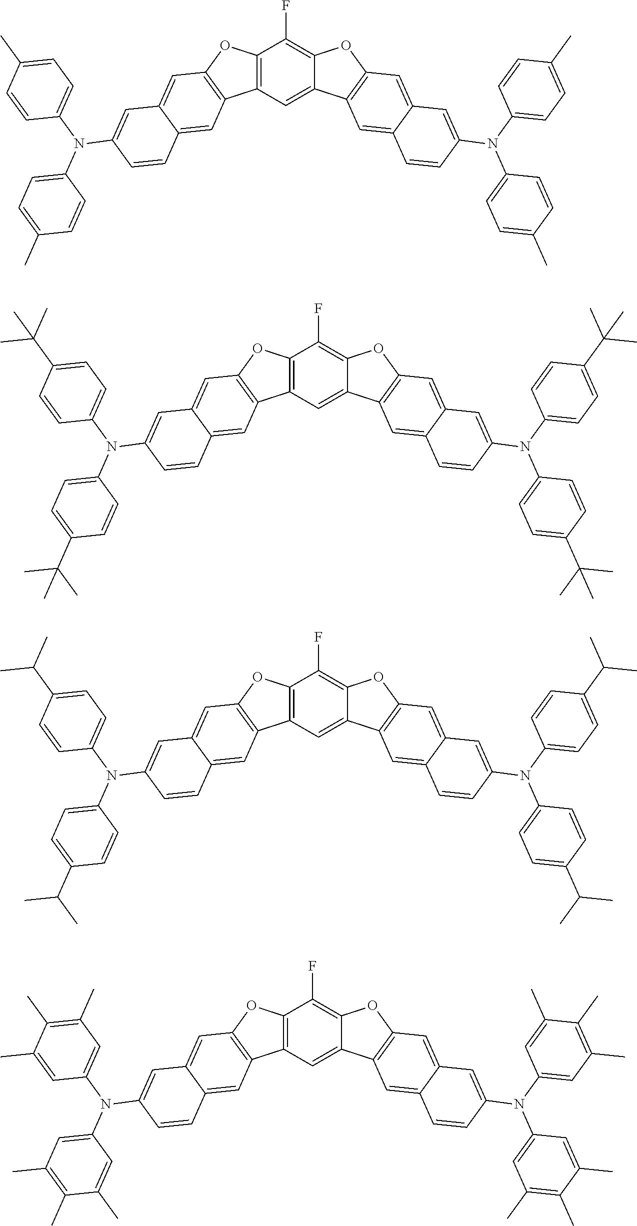

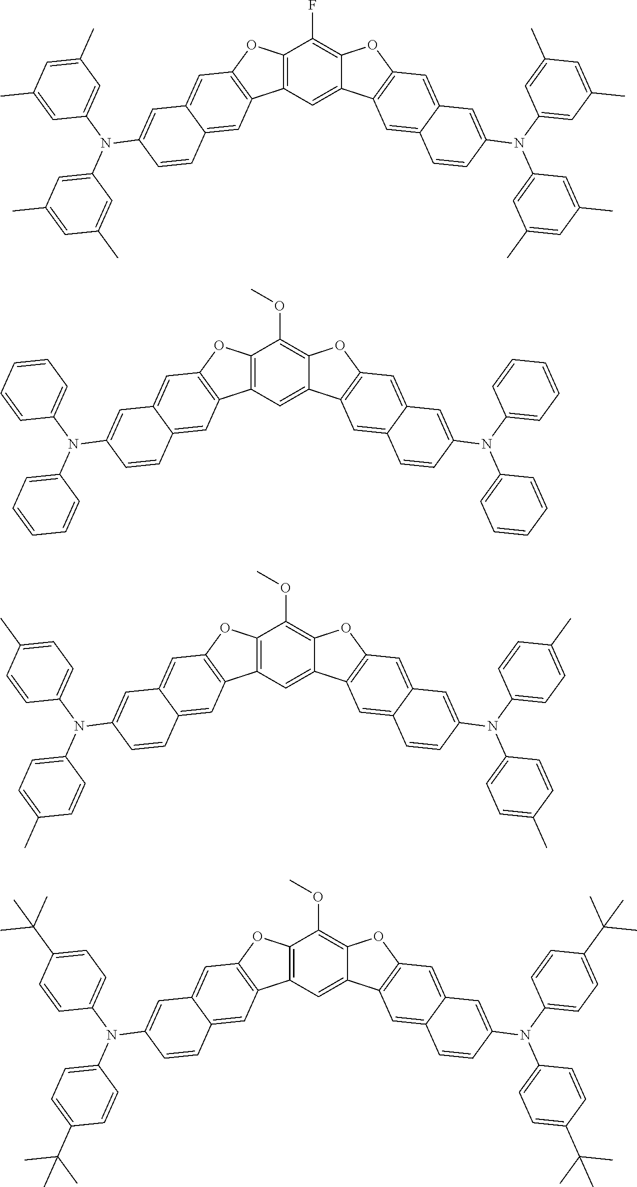

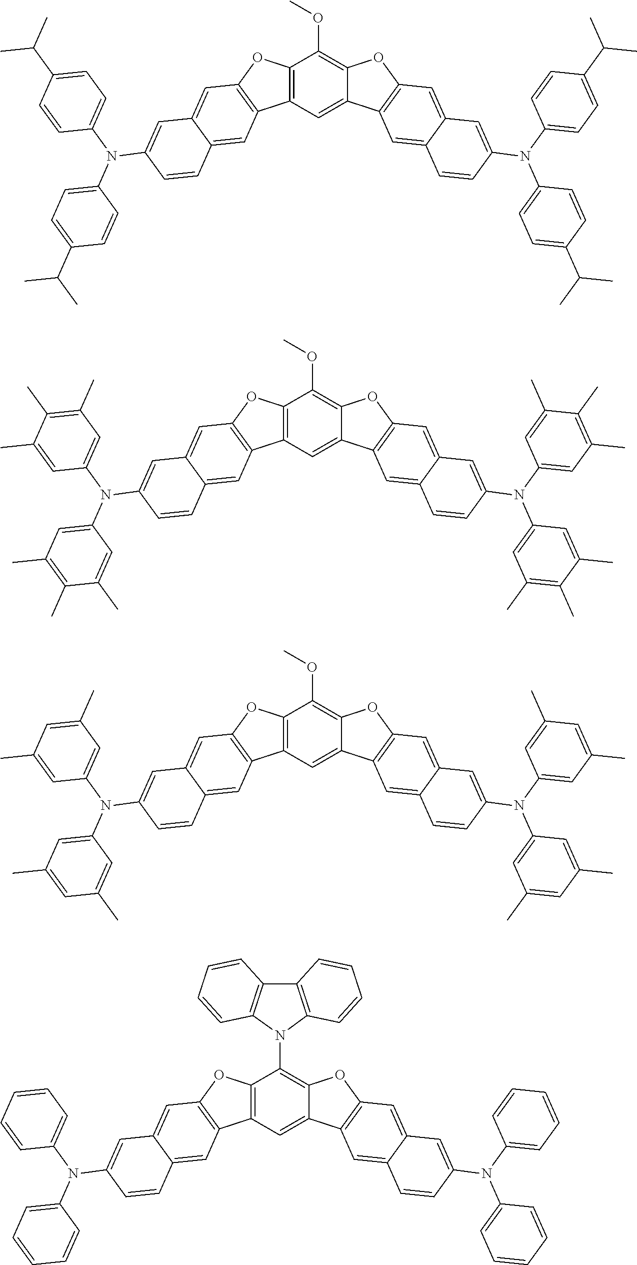

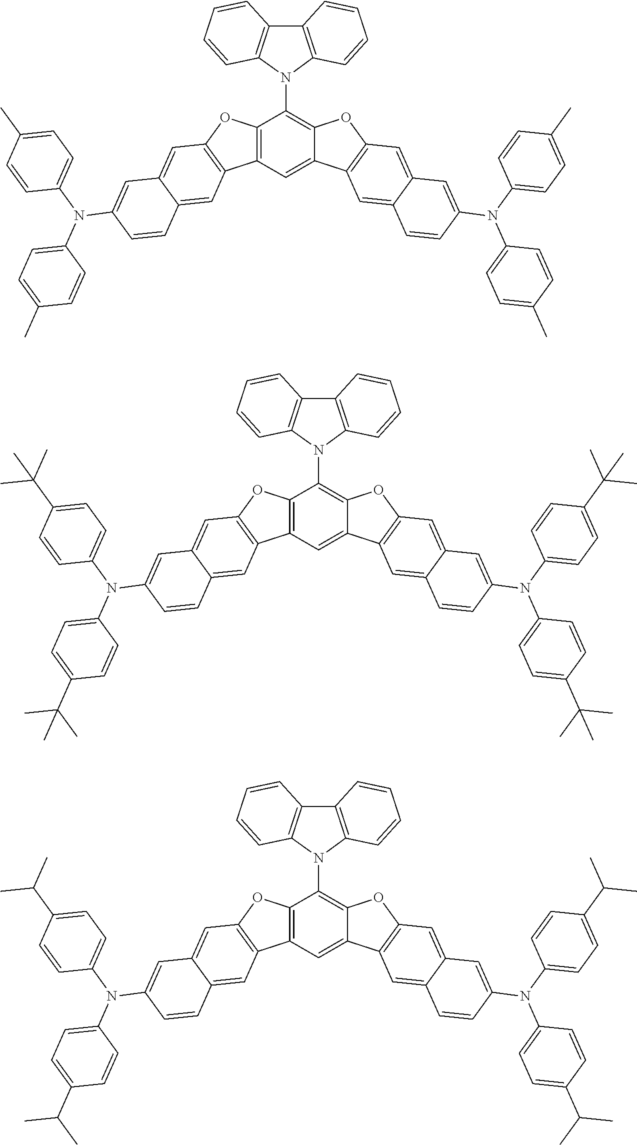

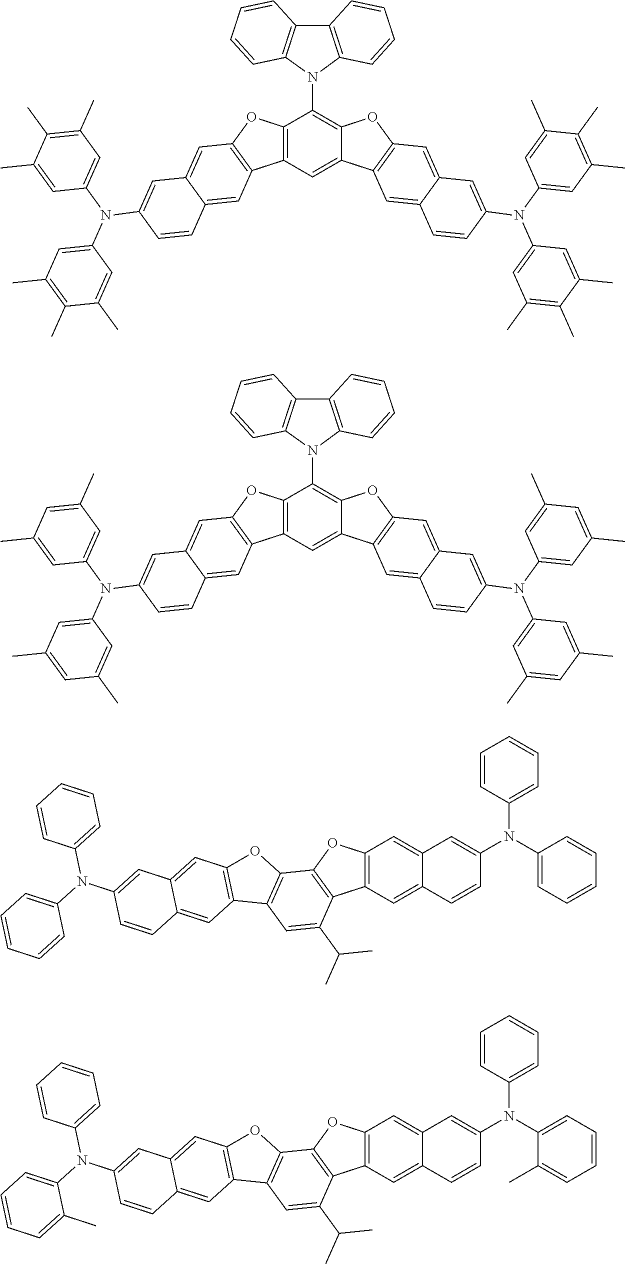

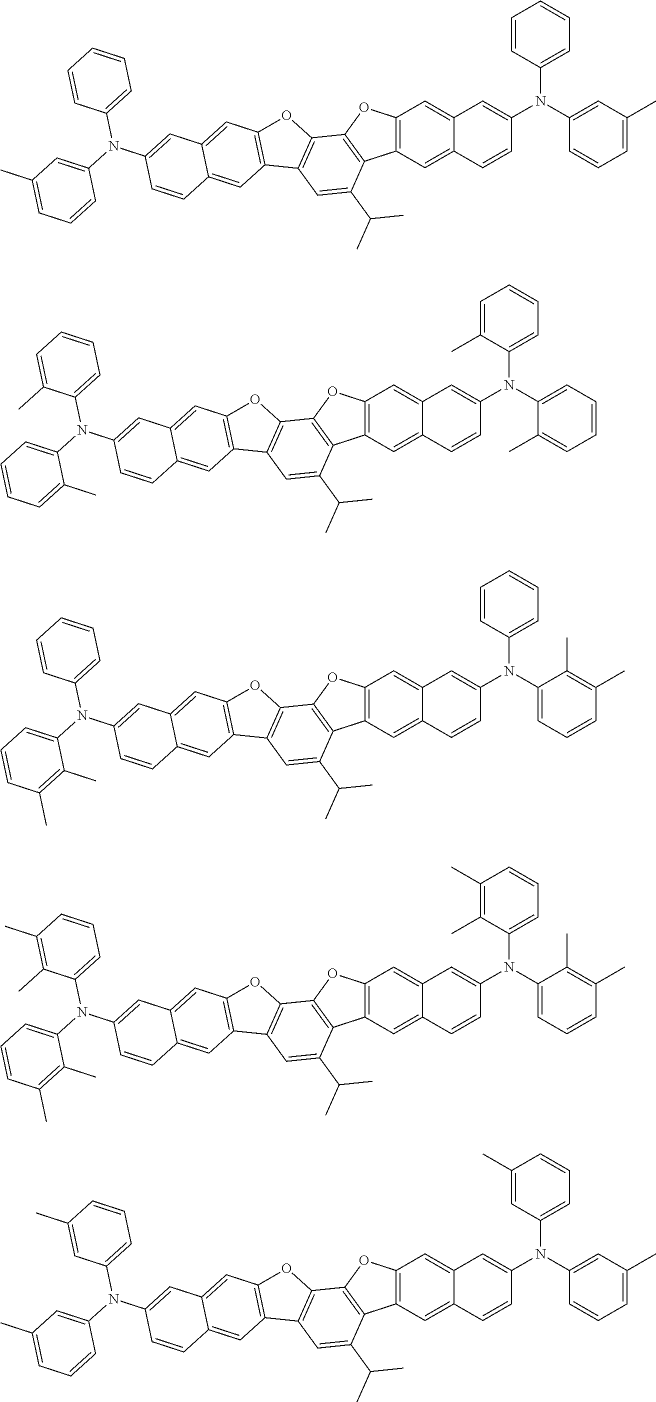

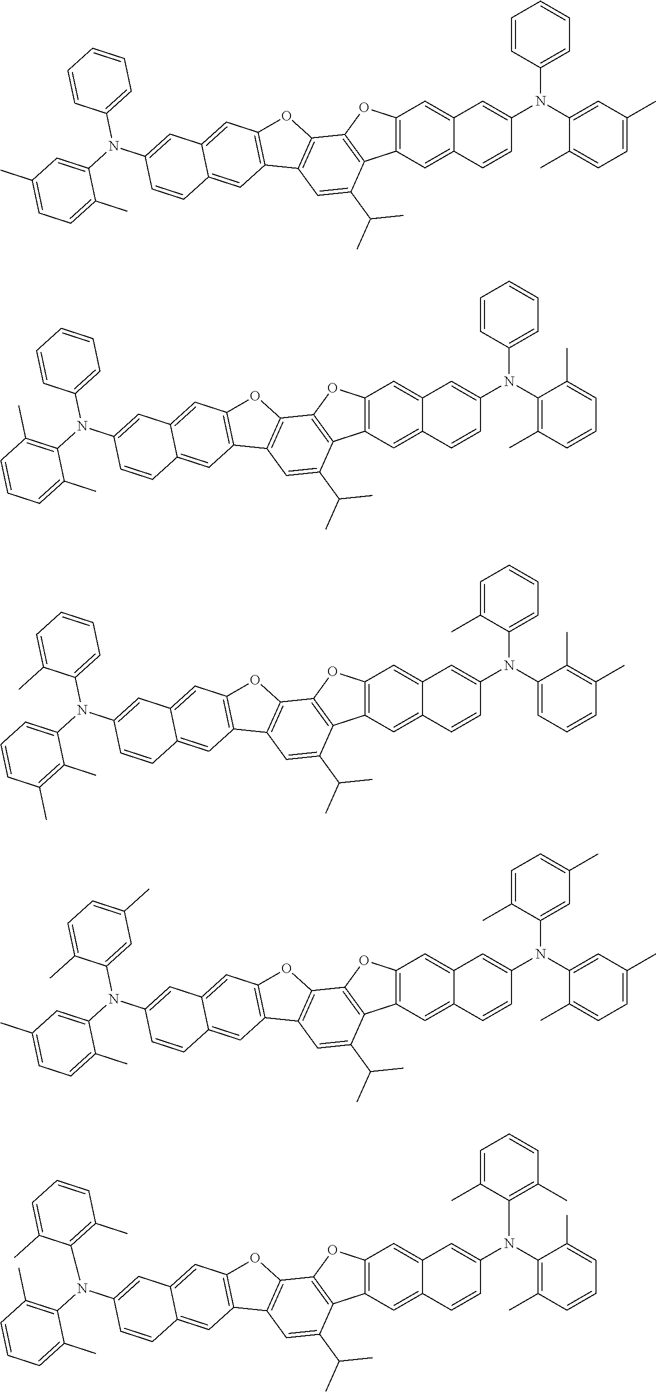

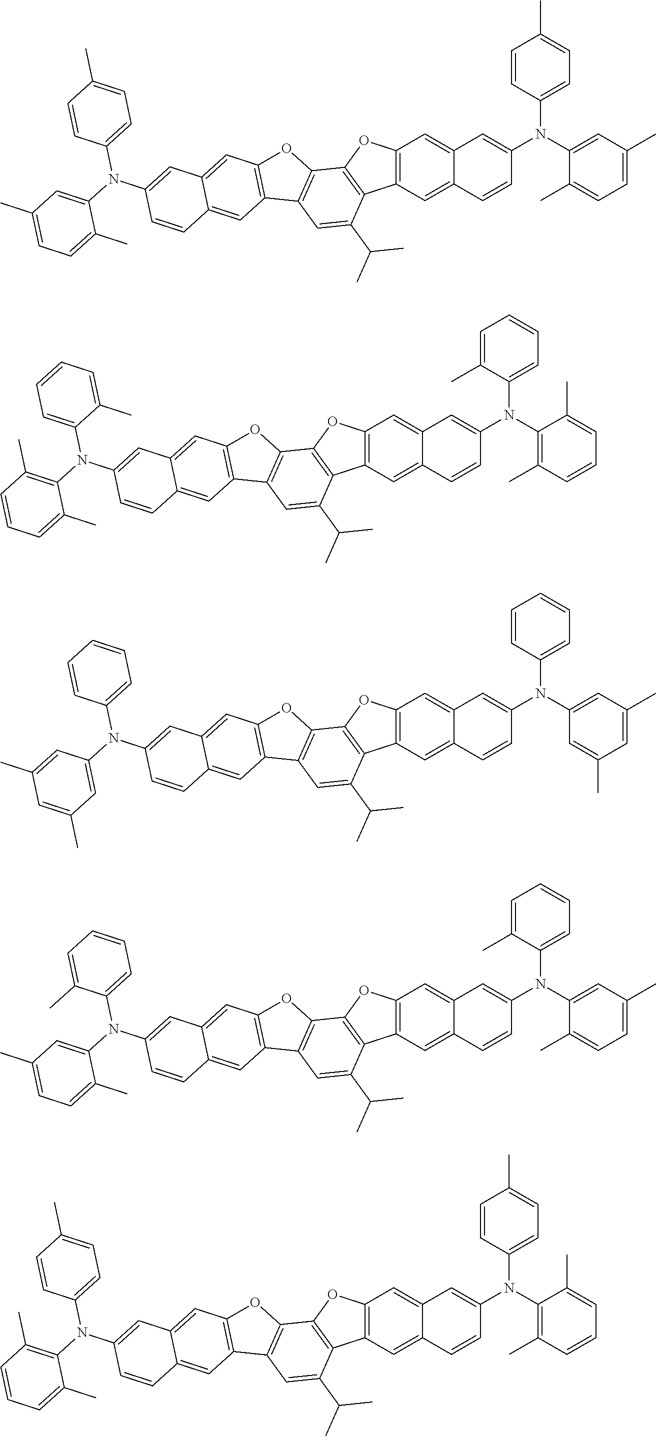

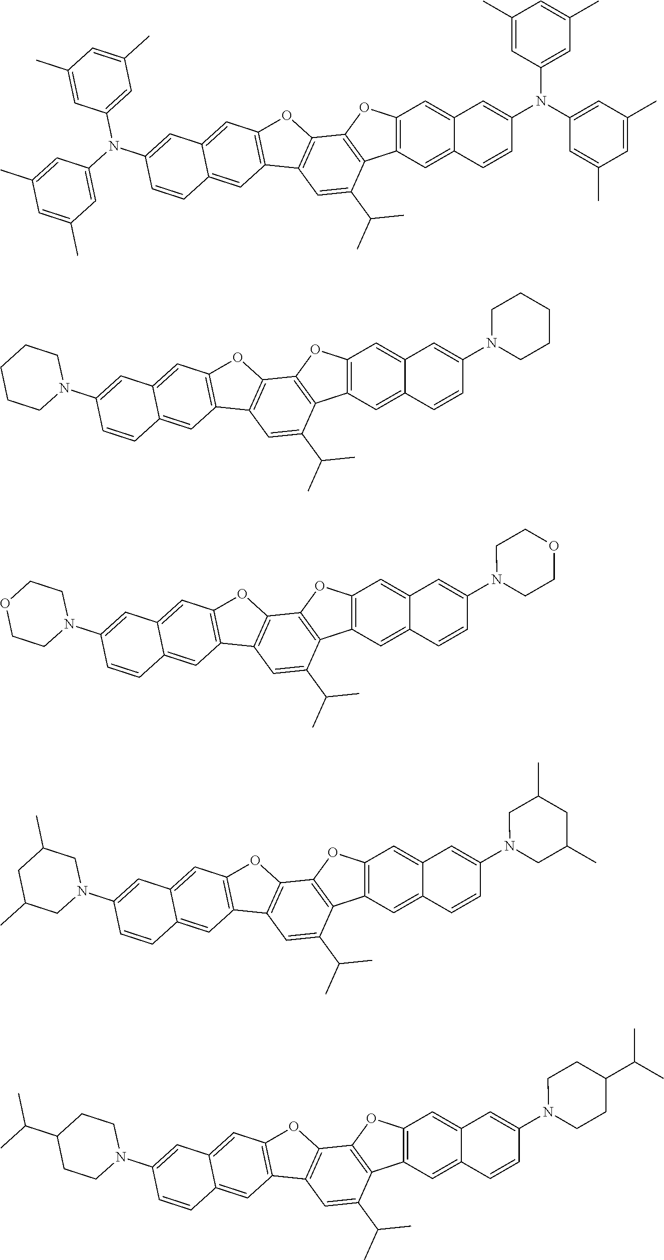

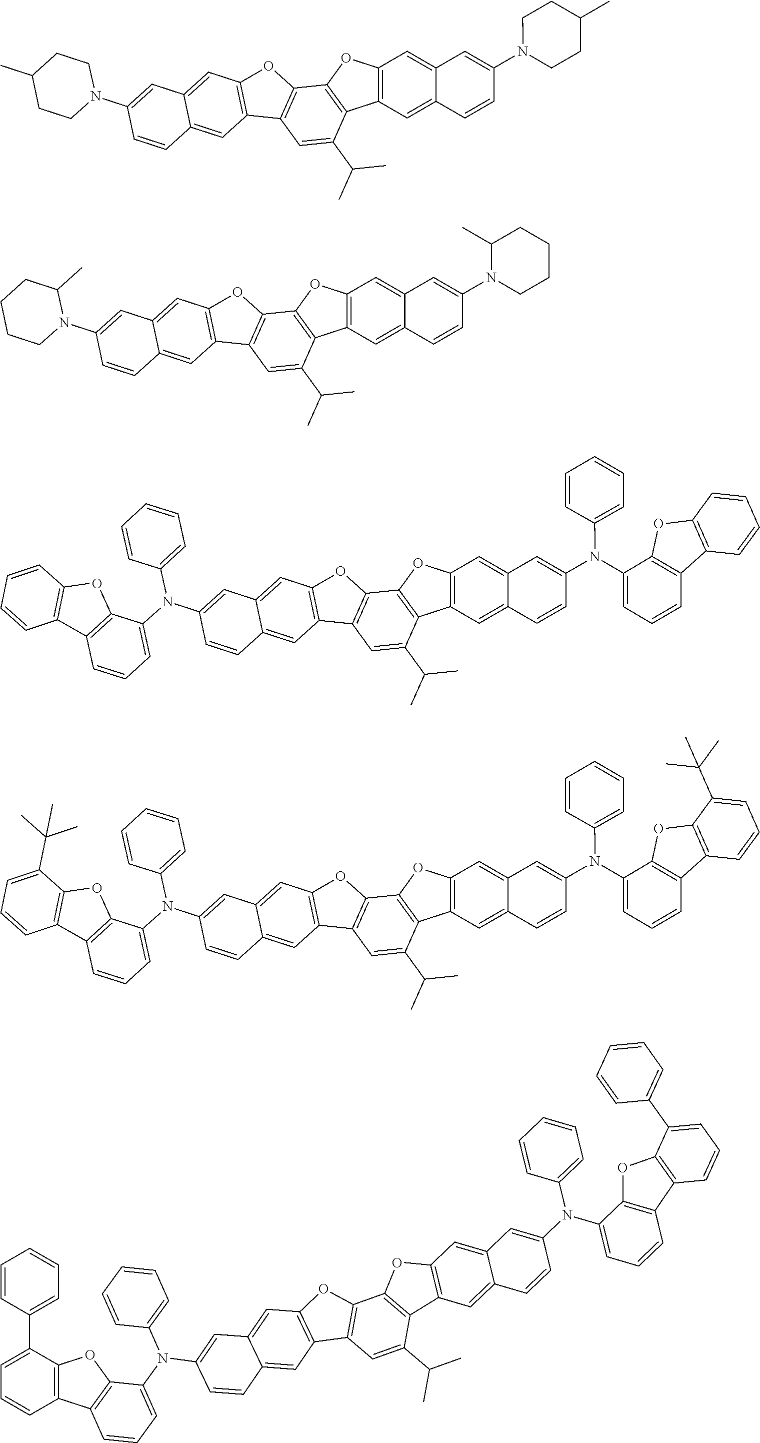

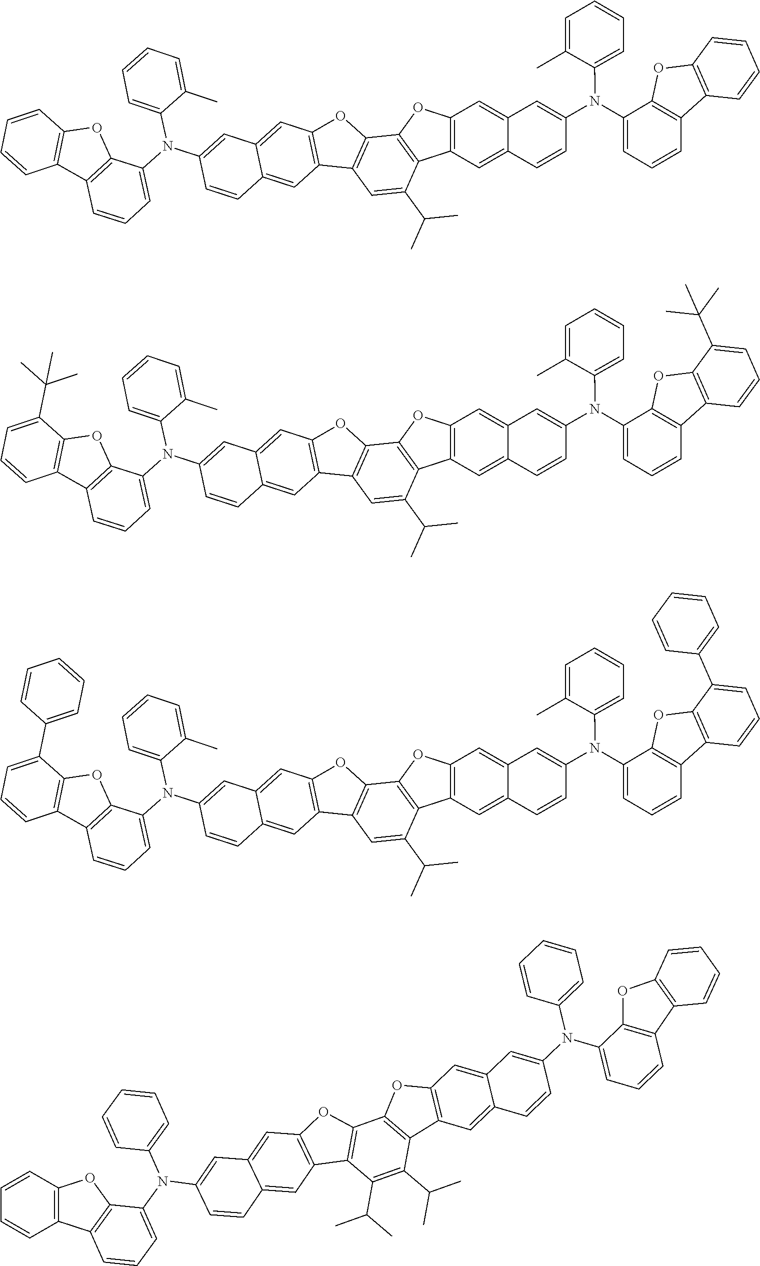

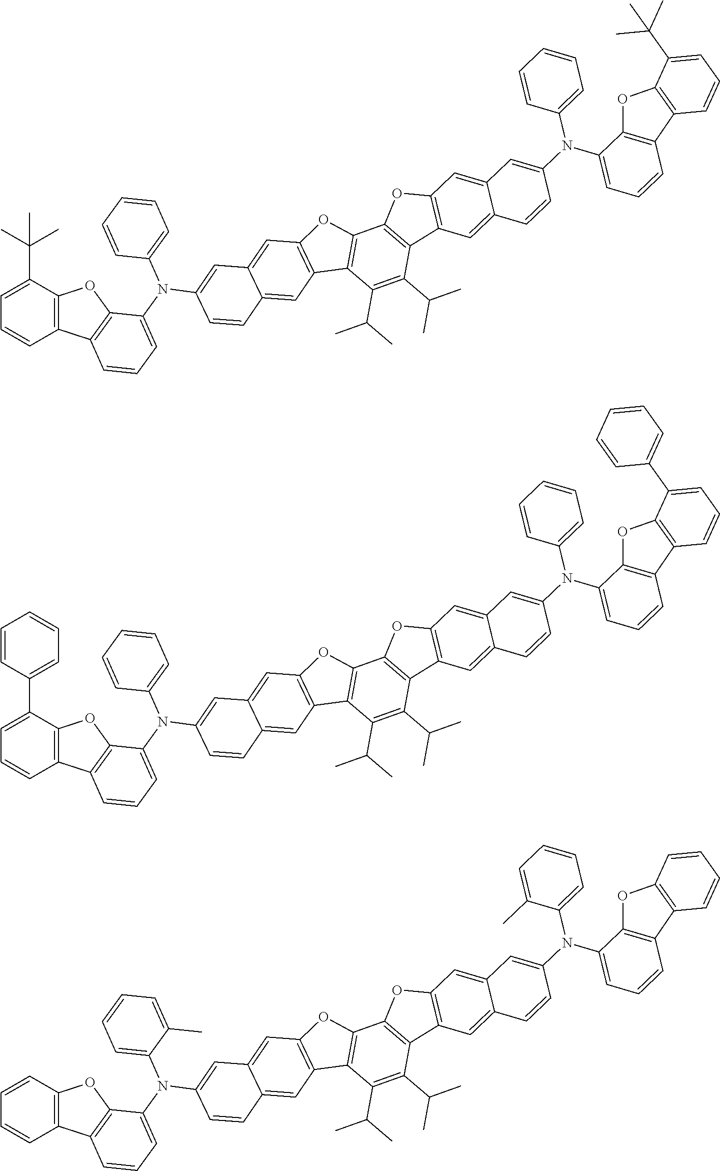 [SMILES: CC(C)c1c(C(C)C)c2c3cc4ccc(N(c5ccccc5)c5cccc6c5oc5c(C(C)(C)C)cccc56)cc4cc3oc2c2oc3cc4cc(N(c5ccccc5)c5cccc6c5oc5c(C(C)(C)C)cccc56)ccc4cc3c12 ; CC(C)c1c(C(C)C)c2c3cc4ccc(N(c5ccccc5)c5cccc6c5oc5c(-c7ccccc7)cccc56)cc4cc3oc2c2oc3cc4cc(N(c5ccccc5)c5cccc6c5oc5c(-c7ccccc7)cccc56)ccc4cc3c12 ; Cc1ccccc1N(c1ccc2cc3c(cc2c1)oc1c2oc4cc5cc(N(c6ccccc6C)c6cccc7c6oc6ccccc67)ccc5cc4c2c(C(C)C)c(C(C)C)c31)c1cccc2c1oc1ccccc12]

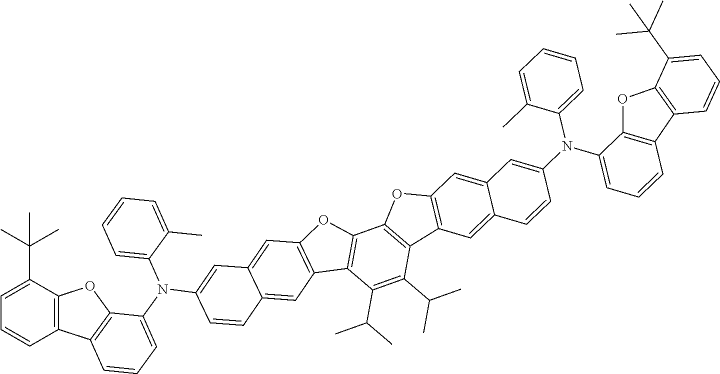 [SMILES: Cc1ccccc1N(c1ccc2cc3c(cc2c1)oc1c2oc4cc5cc(N(c6ccccc6C)c6cccc7c6oc6c(C(C)(C)C)cccc67)ccc5cc4c2c(C(C)C)c(C(C)C)c31)c1cccc2c1oc1c(C(C)(C)C)cccc12]

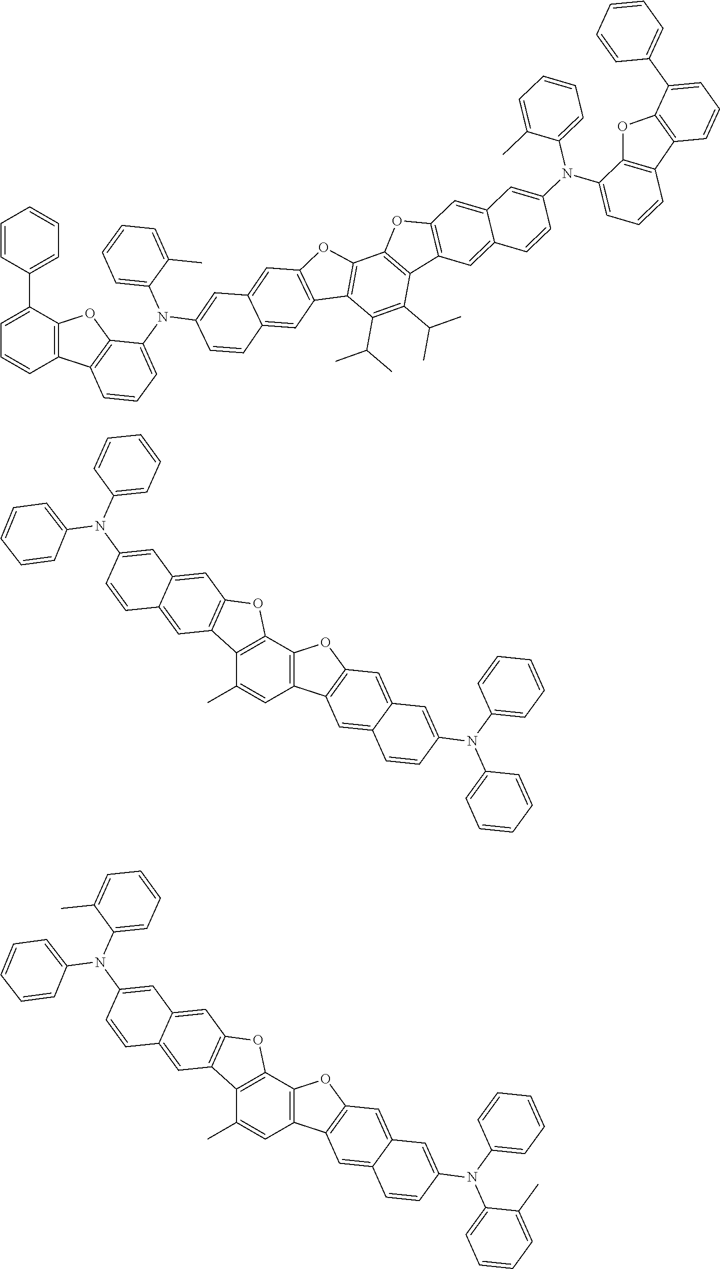

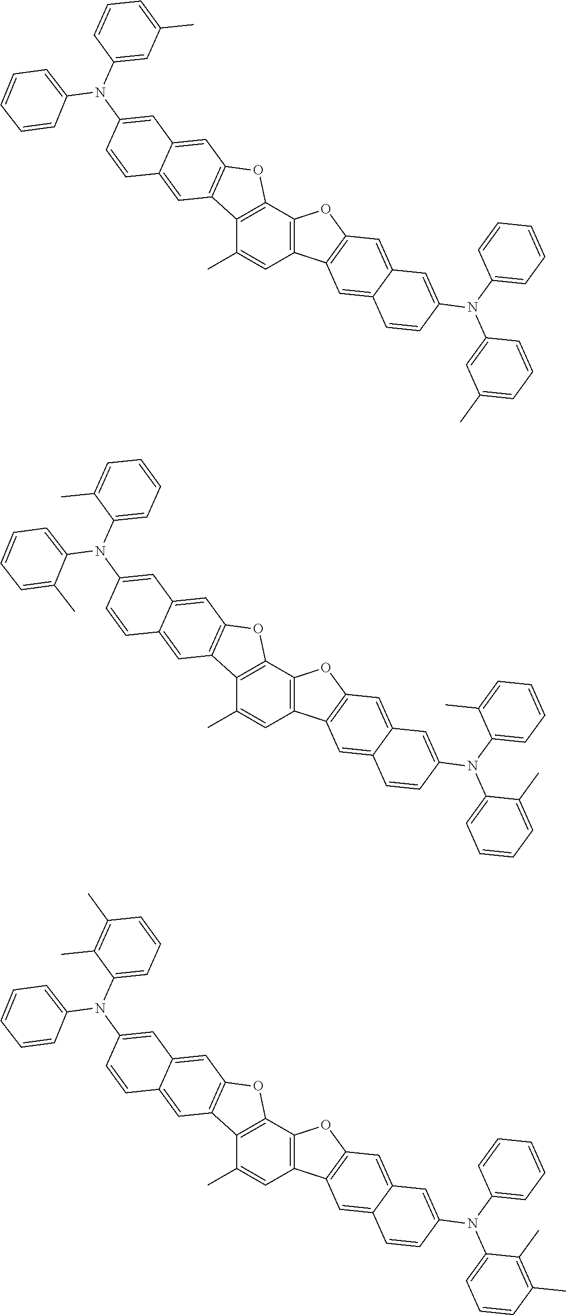 [SMILES: Cc1cccc(N(c2ccccc2)c2ccc3cc4c(cc3c2)oc2c4cc(C)c3c4cc5ccc(N(c6ccccc6)c6cccc(C)c6)cc5cc4oc23)c1 ; Cc1ccccc1N(c1ccc2cc3c(cc2c1)oc1c3cc(C)c2c3cc4ccc(N(c5ccccc5C)c5ccccc5C)cc4cc3oc12)c1ccccc1C ; Cc1cccc(N(c2ccccc2)c2ccc3cc4c(cc3c2)oc2c4cc(C)c3c4cc5ccc(N(c6ccccc6)c6cccc(C)c6C)cc5cc4oc23)c1C]

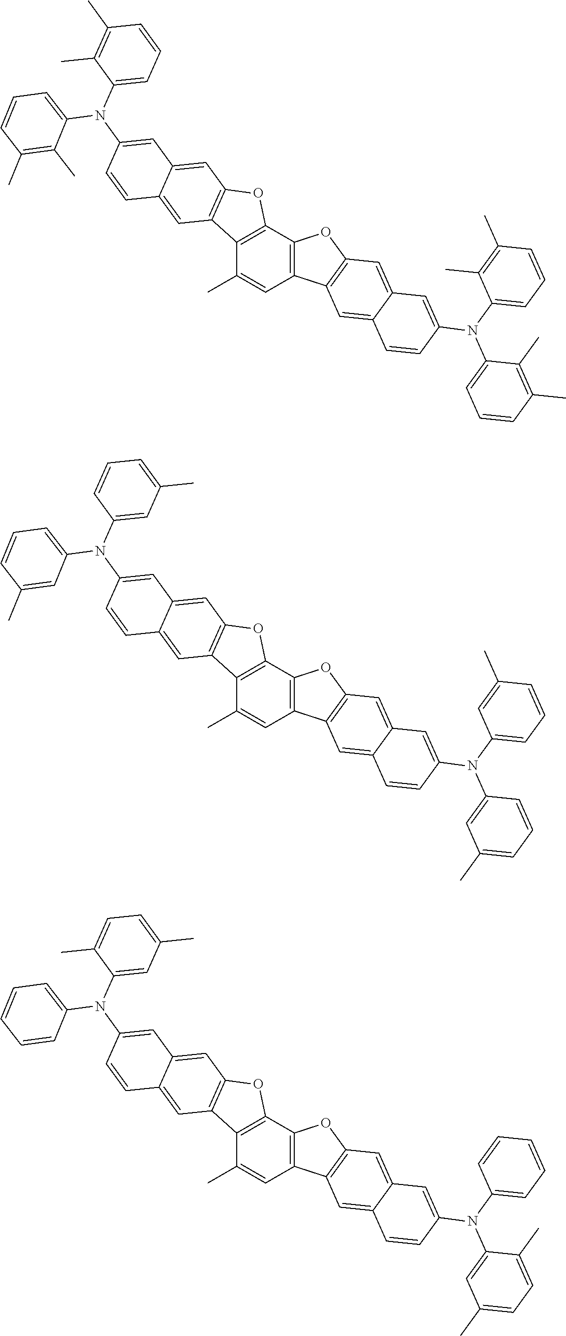 [SMILES: Cc1cccc(N(c2ccc3cc4c(cc3c2)oc2c4cc(C)c3c4cc5ccc(N(c6cccc(C)c6C)c6cccc(C)c6C)cc5cc4oc23)c2cccc(C)c2C)c1C ; Cc1cccc(N(c2cccc(C)c2)c2ccc3cc4c(cc3c2)oc2c4cc(C)c3c4cc5ccc(N(c6cccc(C)c6)c6cccc(C)c6)cc5cc4oc23)c1 ; Cc1ccc(C)c(N(c2ccccc2)c2ccc3cc4c(cc3c2)oc2c4cc(C)c3c4cc5ccc(N(c6ccccc6)c6cc(C)ccc6C)cc5cc4oc23)c1]

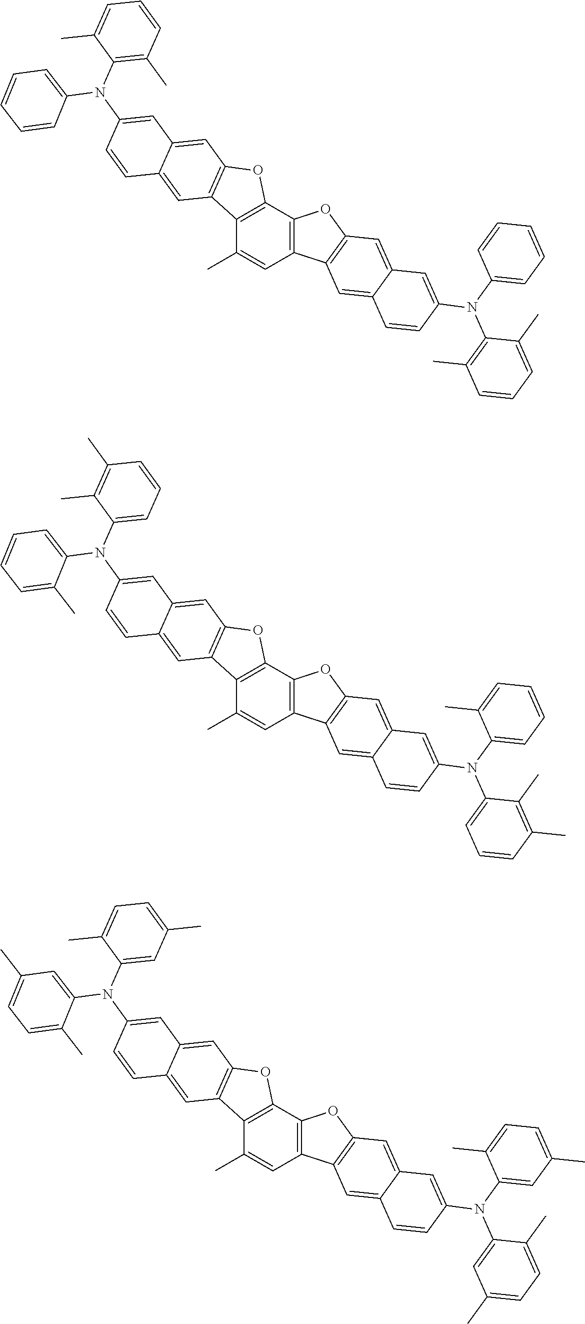

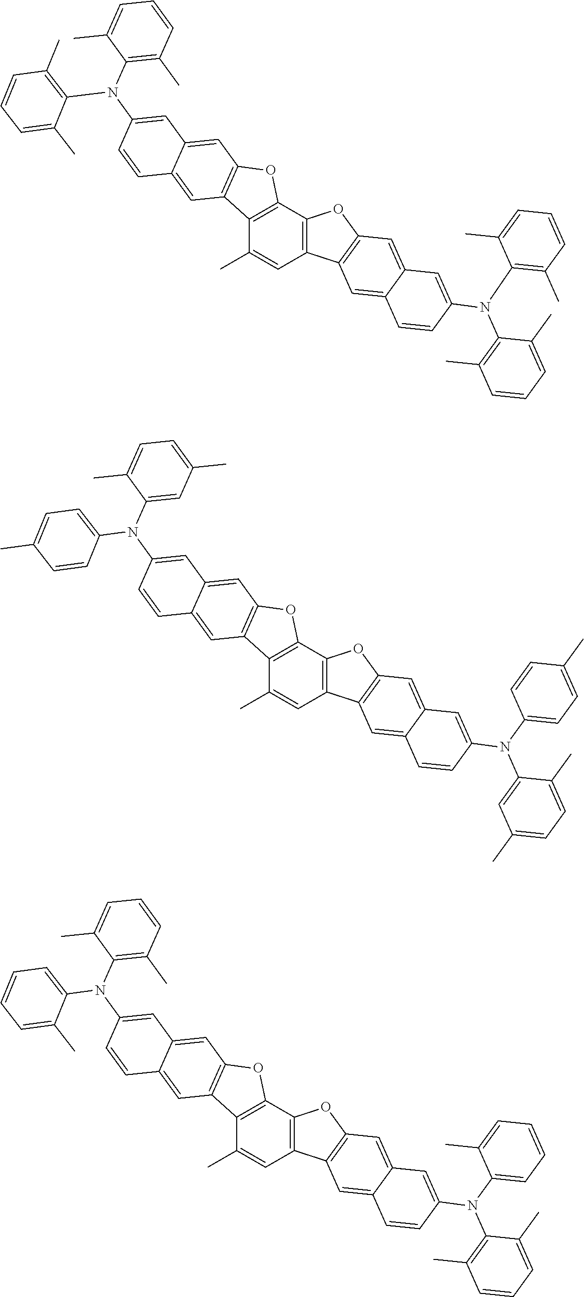

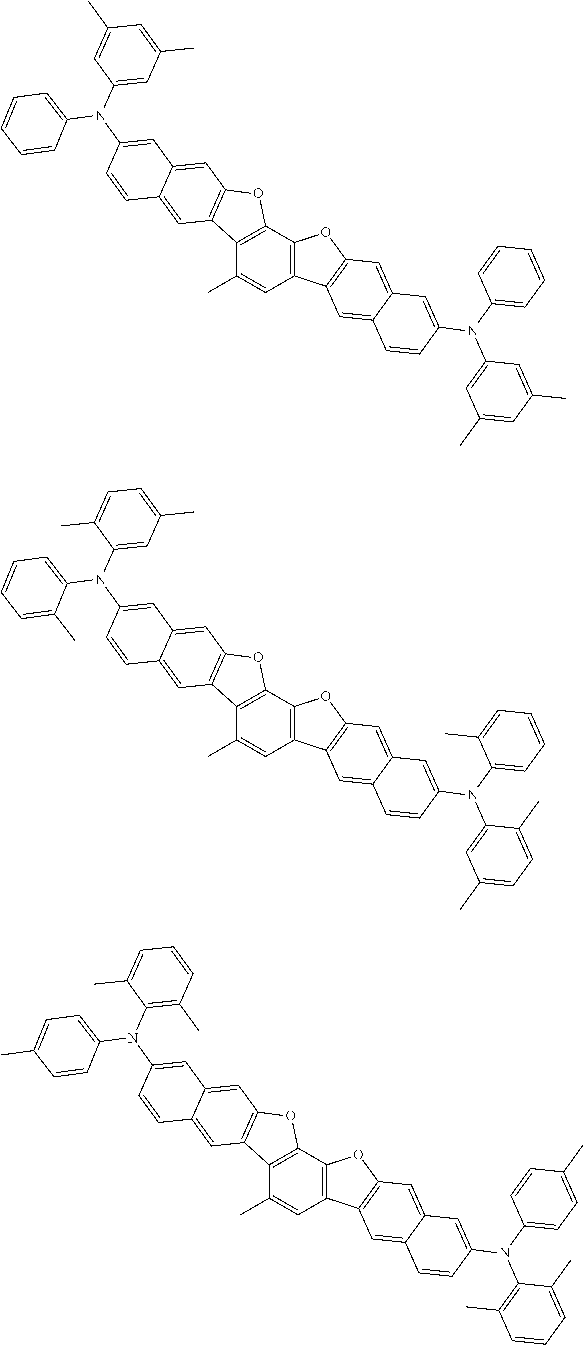

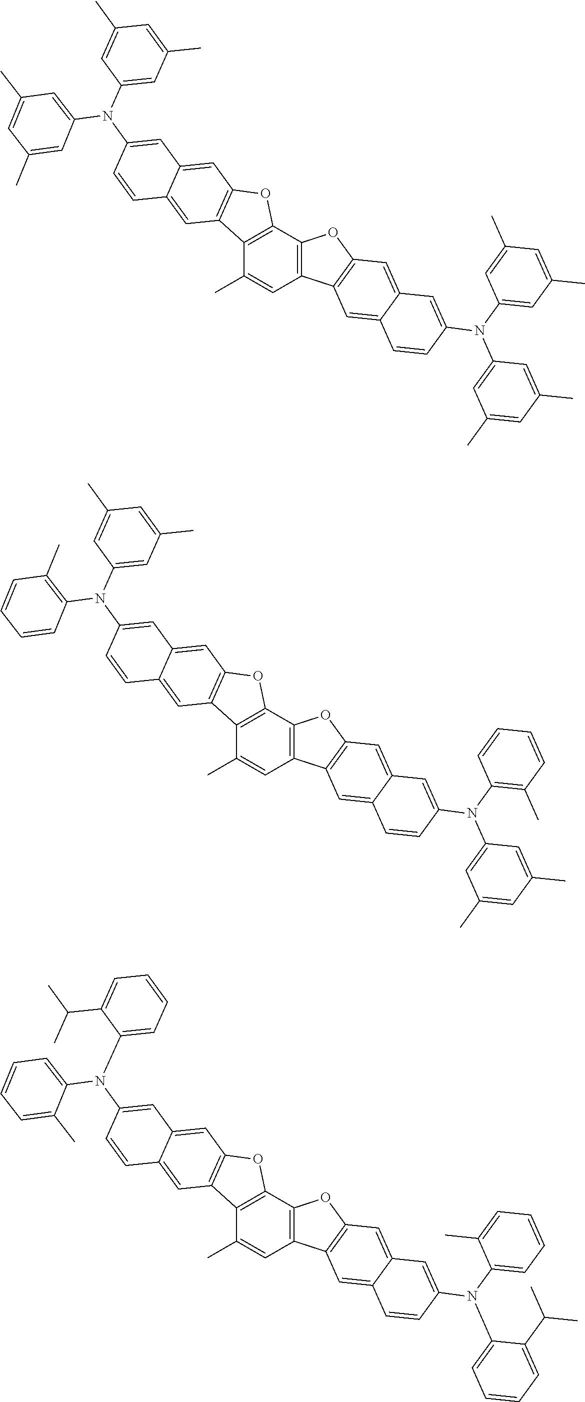

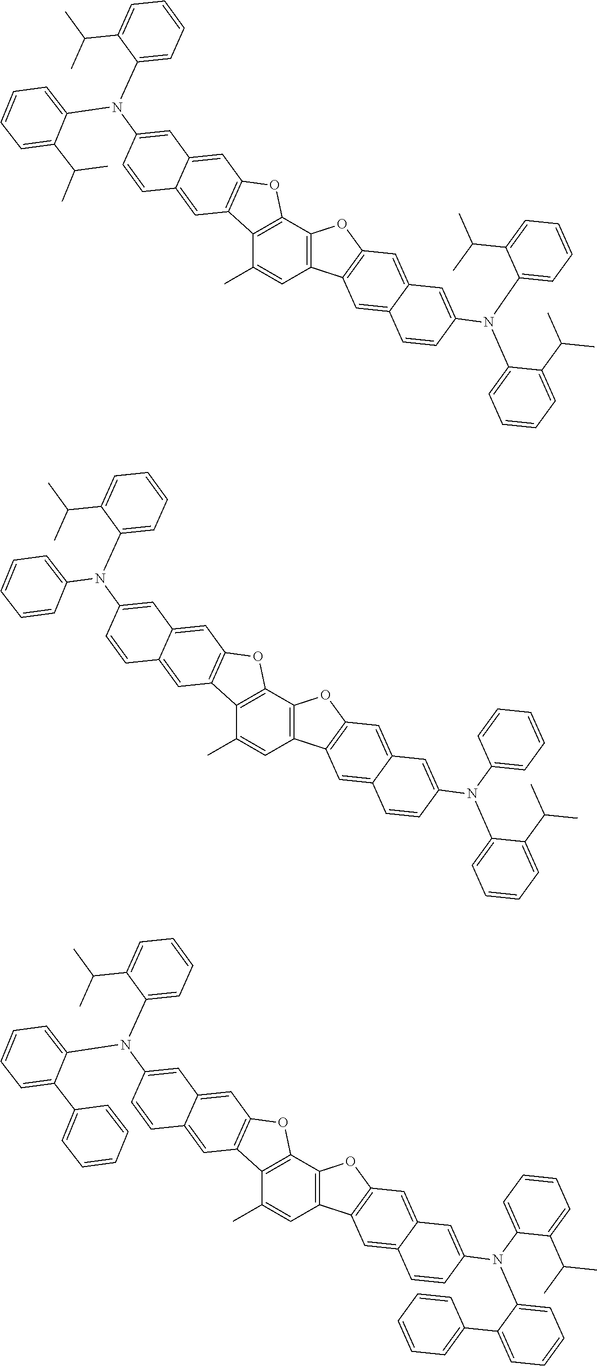 [SMILES: Cc1cc2c3cc4ccc(N(c5ccccc5C(C)C)c5ccccc5C(C)C)cc4cc3oc2c2oc3cc4cc(N(c5ccccc5C(C)C)c5ccccc5C(C)C)ccc4cc3c12 ; Cc1cc2c3cc4ccc(N(c5ccccc5)c5ccccc5C(C)C)cc4cc3oc2c2oc3cc4cc(N(c5ccccc5)c5ccccc5C(C)C)ccc4cc3c12 ; Cc1cc2c3cc4ccc(N(c5ccccc5-c5ccccc5)c5ccccc5C(C)C)cc4cc3oc2c2oc3cc4cc(N(c5ccccc5-c5ccccc5)c5ccccc5C(C)C)ccc4cc3c12]

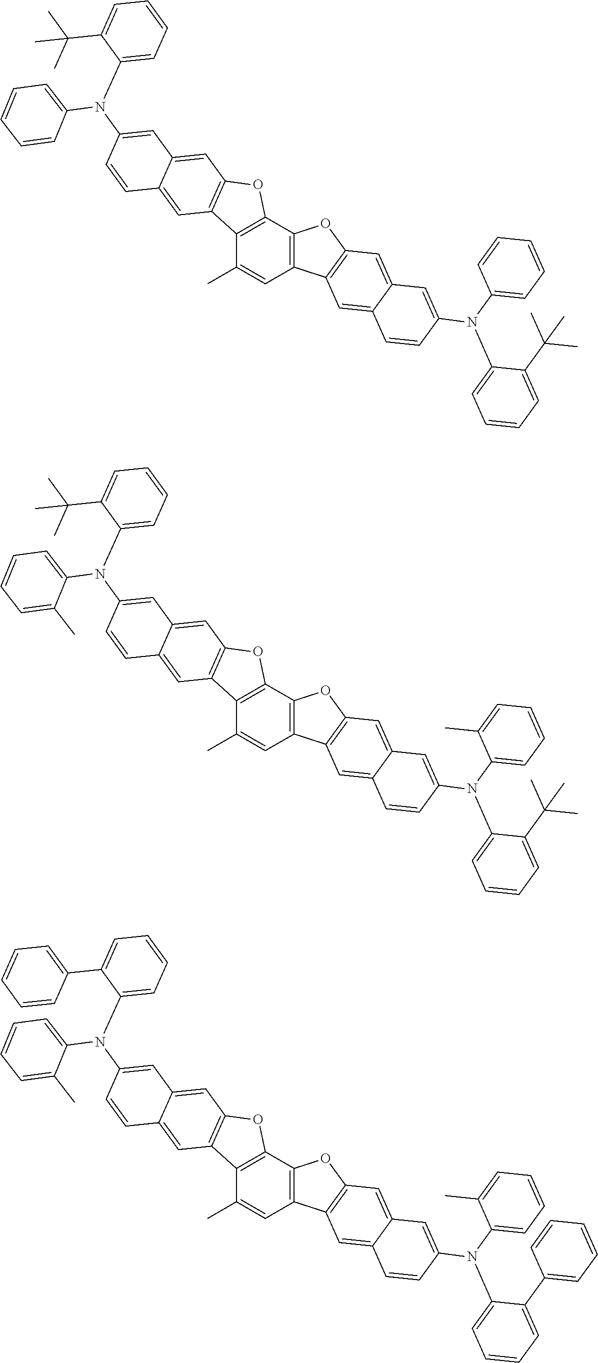 [SMILES: Cc1cc2c3cc4ccc(N(c5ccccc5)c5ccccc5C(C)(C)C)cc4cc3oc2c2oc3cc4cc(N(c5ccccc5)c5ccccc5C(C)(C)C)ccc4cc3c12 ; Cc1ccccc1N(c1ccc2cc3c(cc2c1)oc1c3cc(C)c2c3cc4ccc(N(c5ccccc5C)c5ccccc5C(C)(C)C)cc4cc3oc12)c1ccccc1C(C)(C)C ; Cc1ccccc1N(c1ccc2cc3c(cc2c1)oc1c3cc(C)c2c3cc4ccc(N(c5ccccc5C)c5ccccc5-c5ccccc5)cc4cc3oc12)c1ccccc1-c1ccccc1]

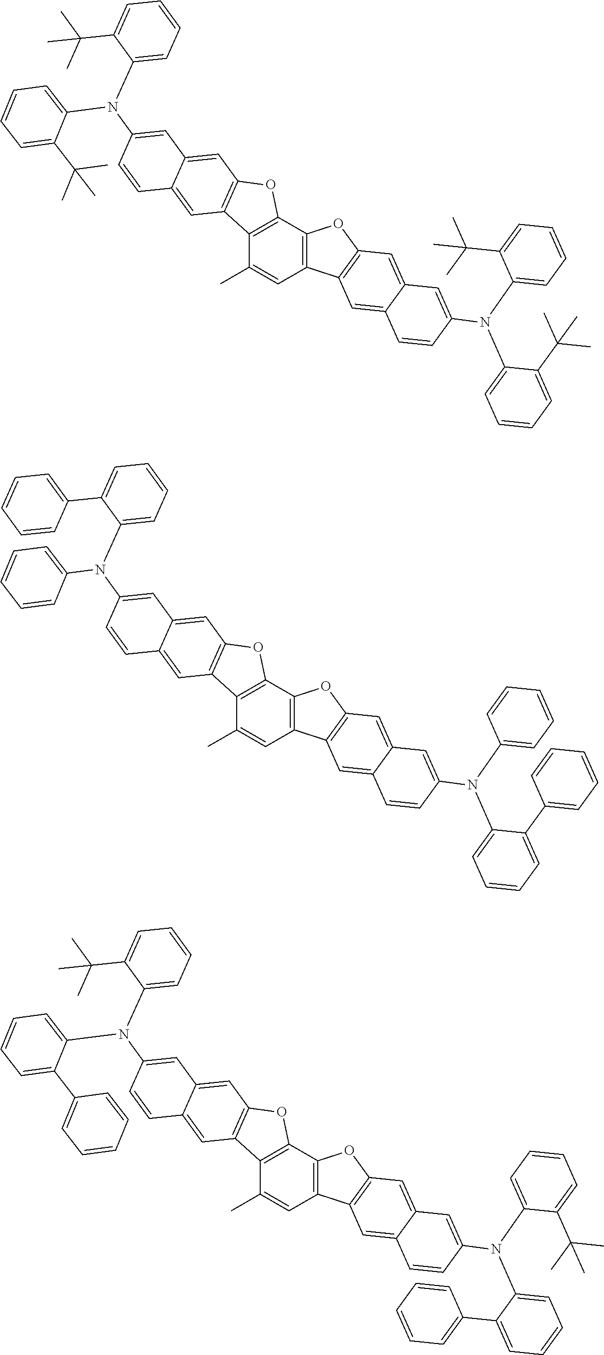 [SMILES: Cc1cc2c3cc4ccc(N(c5ccccc5C(C)(C)C)c5ccccc5C(C)(C)C)cc4cc3oc2c2oc3cc4cc(N(c5ccccc5C(C)(C)C)c5ccccc5C(C)(C)C)ccc4cc3c12 ; Cc1cc2c3cc4ccc(N(c5ccccc5)c5ccccc5-c5ccccc5)cc4cc3oc2c2oc3cc4cc(N(c5ccccc5)c5ccccc5-c5ccccc5)ccc4cc3c12 ; Cc1cc2c3cc4ccc(N(c5ccccc5-c5ccccc5)c5ccccc5C(C)(C)C)cc4cc3oc2c2oc3cc4cc(N(c5ccccc5-c5ccccc5)c5ccccc5C(C)(C)C)ccc4cc3c12]

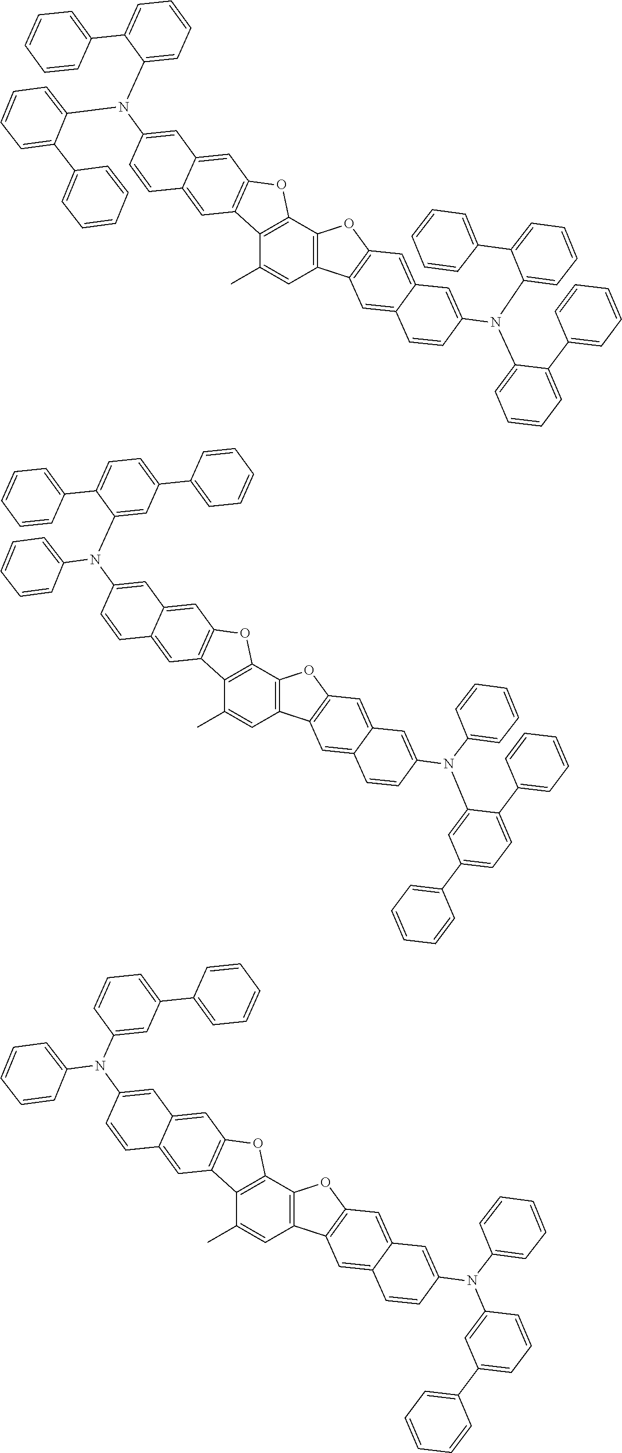 [SMILES: Cc1cc2c3cc4ccc(N(c5ccccc5-c5ccccc5)c5ccccc5-c5ccccc5)cc4cc3oc2c2oc3cc4cc(N(c5ccccc5-c5ccccc5)c5ccccc5-c5ccccc5)ccc4cc3c12 ; Cc1cc2c3cc4ccc(N(c5ccccc5)c5cc(-c6ccccc6)ccc5-c5ccccc5)cc4cc3oc2c2oc3cc4cc(N(c5ccccc5)c5cc(-c6ccccc6)ccc5-c5ccccc5)ccc4cc3c12 ; Cc1cc2c3cc4ccc(N(c5ccccc5)c5cccc(-c6ccccc6)c5)cc4cc3oc2c2oc3cc4cc(N(c5ccccc5)c5cccc(-c6ccccc6)c5)ccc4cc3c12]

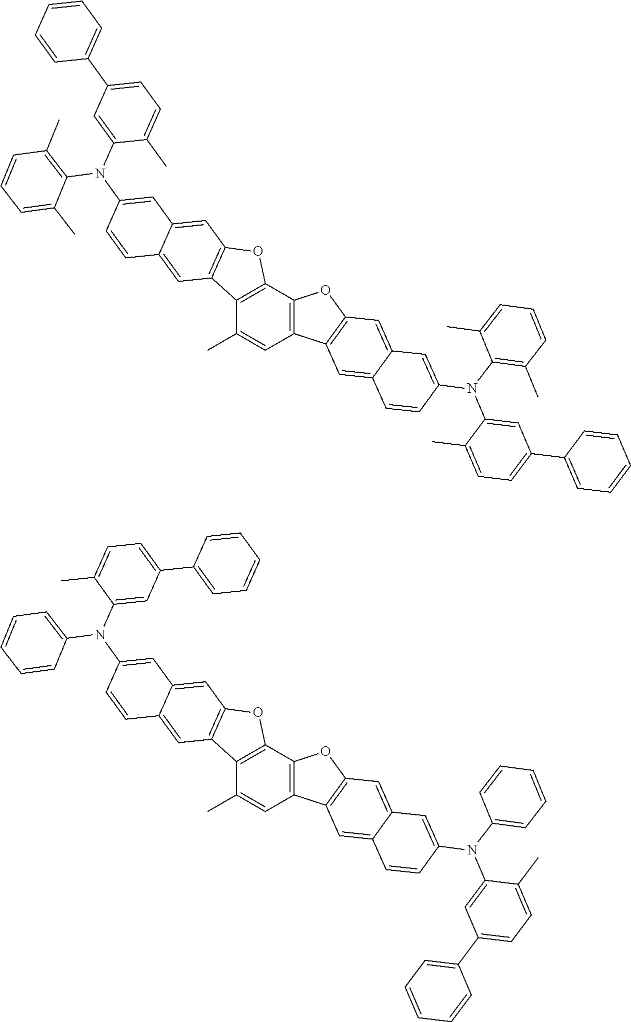 [SMILES: Cc1ccc(-c2ccccc2)cc1N(c1ccc2cc3c(cc2c1)oc1c3cc(C)c2c3cc4ccc(N(c5cc(-c6ccccc6)ccc5C)c5c(C)cccc5C)cc4cc3oc12)c1c(C)cccc1C ; Cc1ccc(-c2ccccc2)cc1N(c1ccccc1)c1ccc2cc3c(cc2c1)oc1c3cc(C)c2c3cc4ccc(N(c5ccccc5)c5cc(-c6ccccc6)ccc5C)cc4cc3oc12]

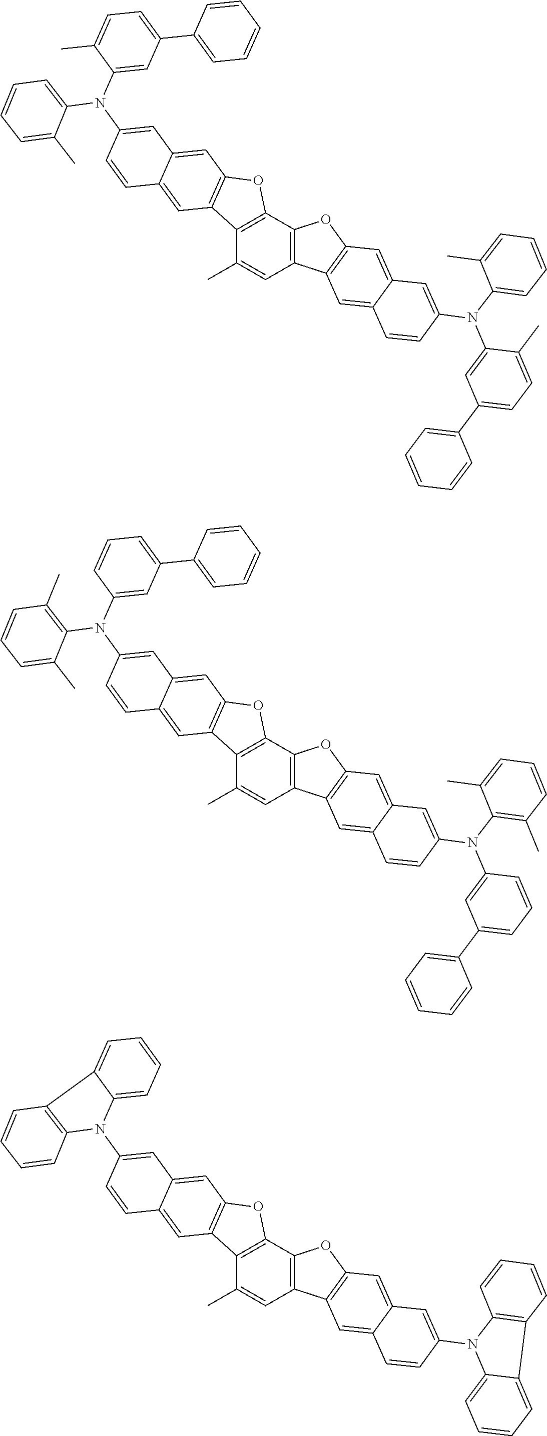 [SMILES: Cc1ccccc1N(c1ccc2cc3c(cc2c1)oc1c3cc(C)c2c3cc4ccc(N(c5ccccc5C)c5cc(-c6ccccc6)ccc5C)cc4cc3oc12)c1cc(-c2ccccc2)ccc1C ; Cc1cccc(C)c1N(c1cccc(-c2ccccc2)c1)c1ccc2cc3c(cc2c1)oc1c3cc(C)c2c3cc4ccc(N(c5cccc(-c6ccccc6)c5)c5c(C)cccc5C)cc4cc3oc12 ; Cc1cc2c3cc4ccc(-n5c6ccccc6c6ccccc65)cc4cc3oc2c2oc3cc4cc(-n5c6ccccc6c6ccccc65)ccc4cc3c12]

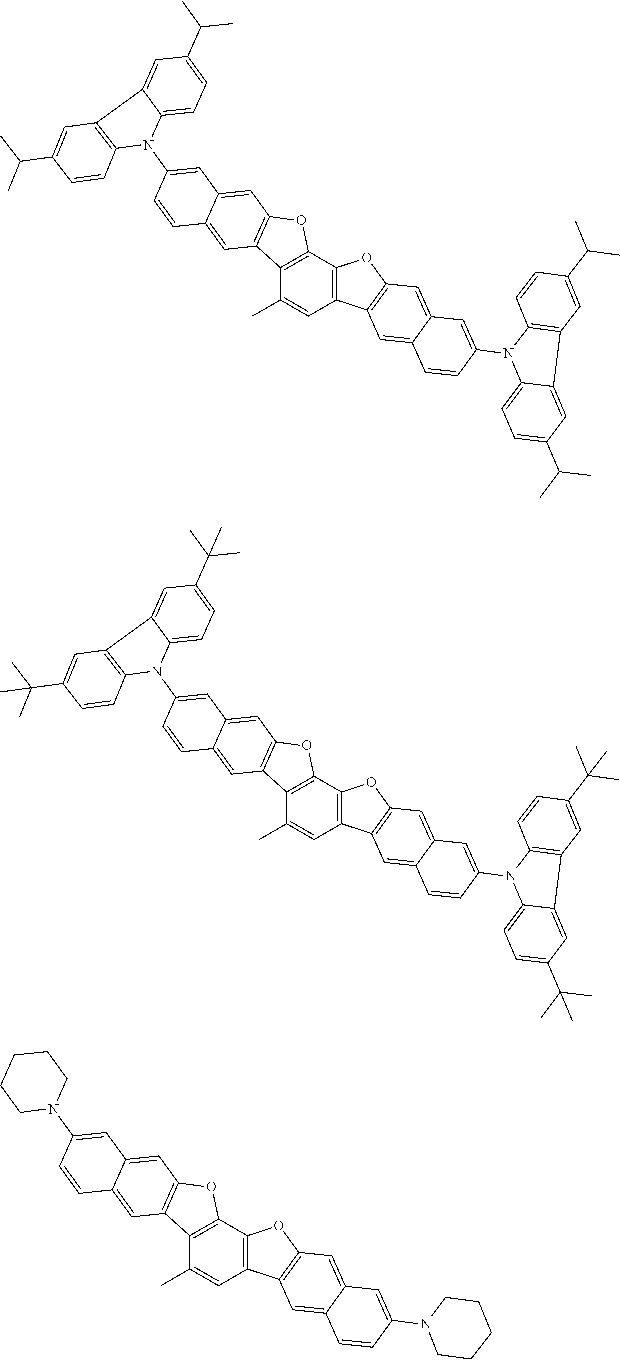

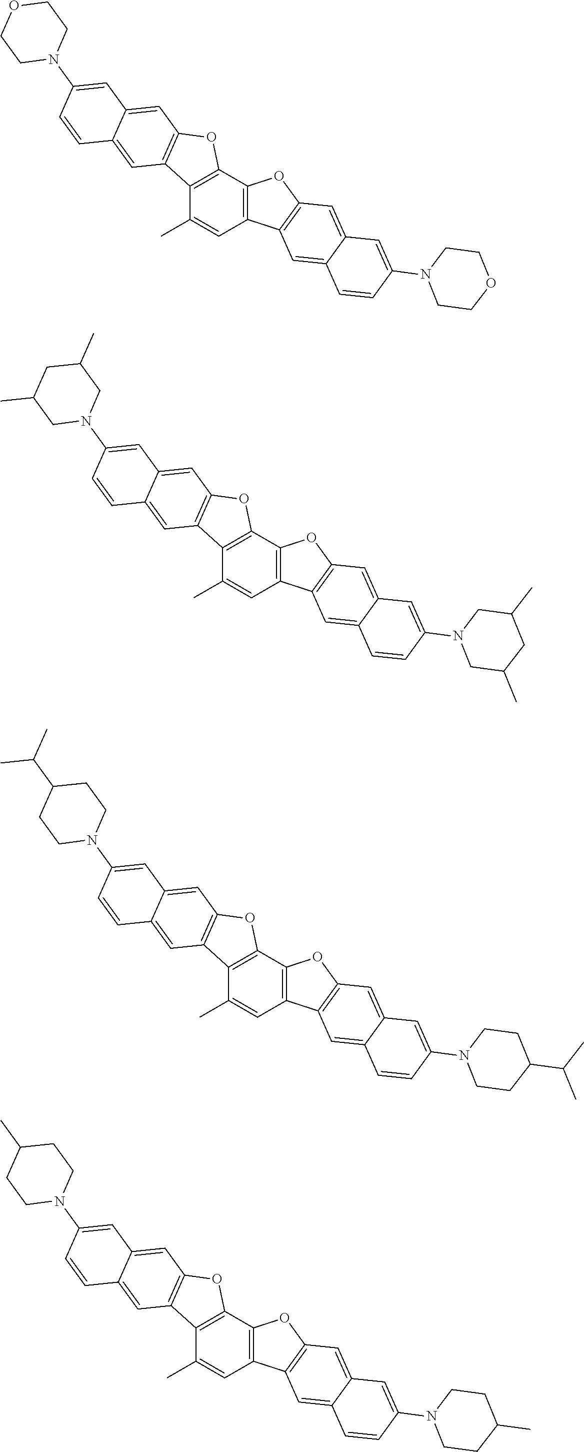

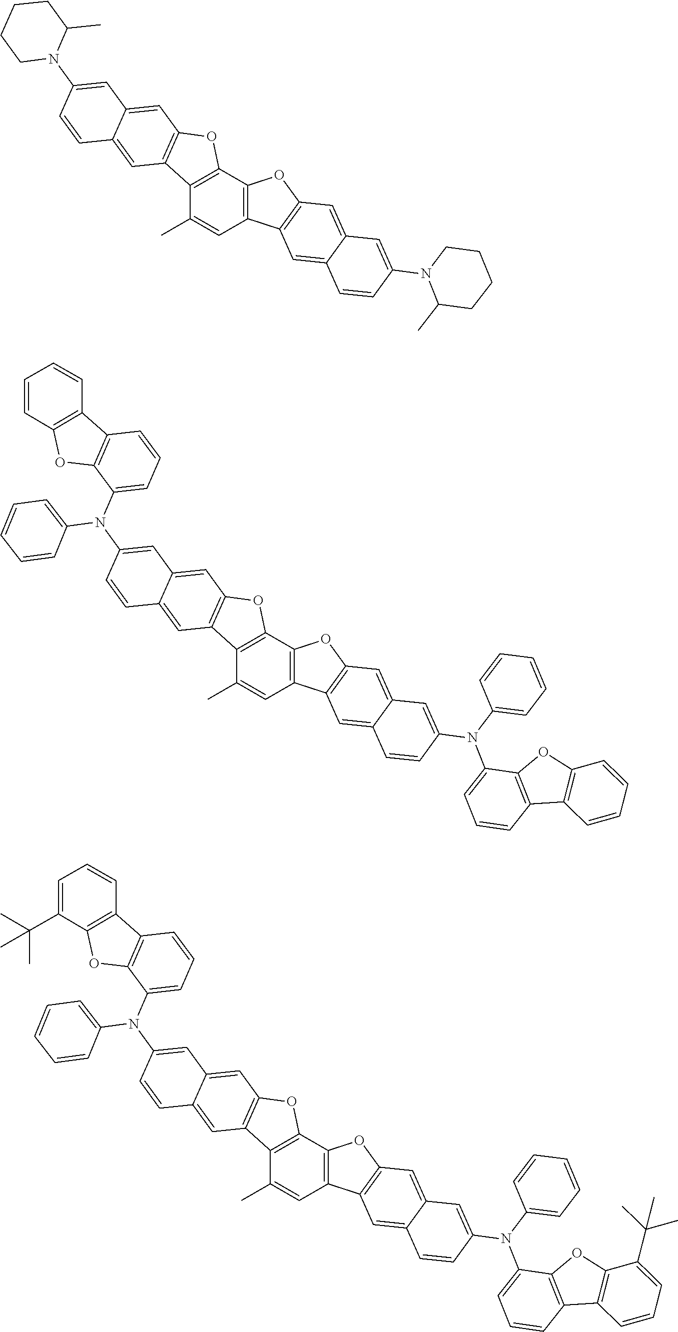 [SMILES: Cc1cc2c3cc4ccc(N5CCCCC5C)cc4cc3oc2c2oc3cc4cc(N5CCCCC5C)ccc4cc3c12 ; Cc1cc2c3cc4ccc(N(c5ccccc5)c5cccc6c5oc5ccccc56)cc4cc3oc2c2oc3cc4cc(N(c5ccccc5)c5cccc6c5oc5ccccc56)ccc4cc3c12 ; Cc1cc2c3cc4ccc(N(c5ccccc5)c5cccc6c5oc5c(C(C)(C)C)cccc56)cc4cc3oc2c2oc3cc4cc(N(c5ccccc5)c5cccc6c5oc5c(C(C)(C)C)cccc56)ccc4cc3c12]

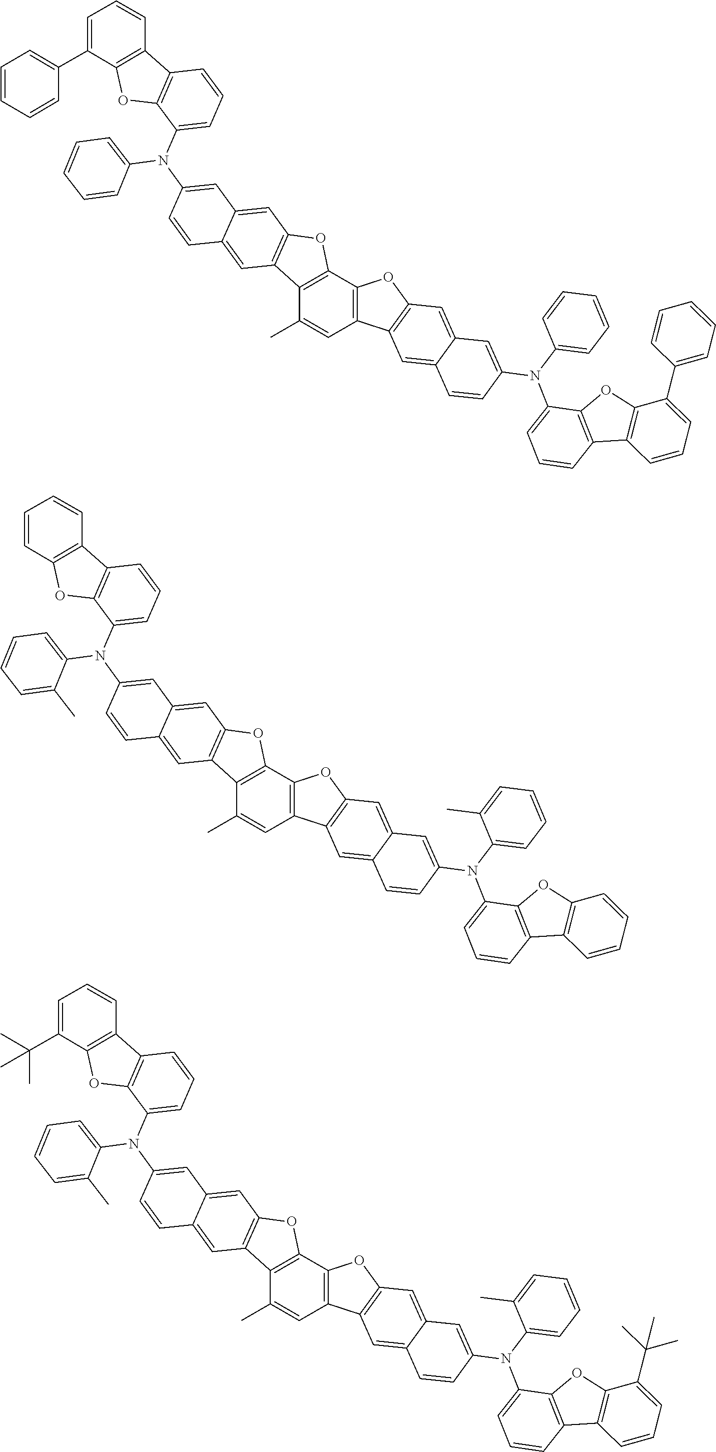 [SMILES: Cc1cc2c3cc4ccc(N(c5ccccc5)c5cccc6c5oc5c(-c7ccccc7)cccc56)cc4cc3oc2c2oc3cc4cc(N(c5ccccc5)c5cccc6c5oc5c(-c7ccccc7)cccc56)ccc4cc3c12 ; Cc1ccccc1N(c1ccc2cc3c(cc2c1)oc1c3cc(C)c2c3cc4ccc(N(c5ccccc5C)c5cccc6c5oc5ccccc56)cc4cc3oc12)c1cccc2c1oc1ccccc12 ; Cc1ccccc1N(c1ccc2cc3c(cc2c1)oc1c3cc(C)c2c3cc4ccc(N(c5ccccc5C)c5cccc6c5oc5c(C(C)(C)C)cccc56)cc4cc3oc12)c1cccc2c1oc1c(C(C)(C)C)cccc12]

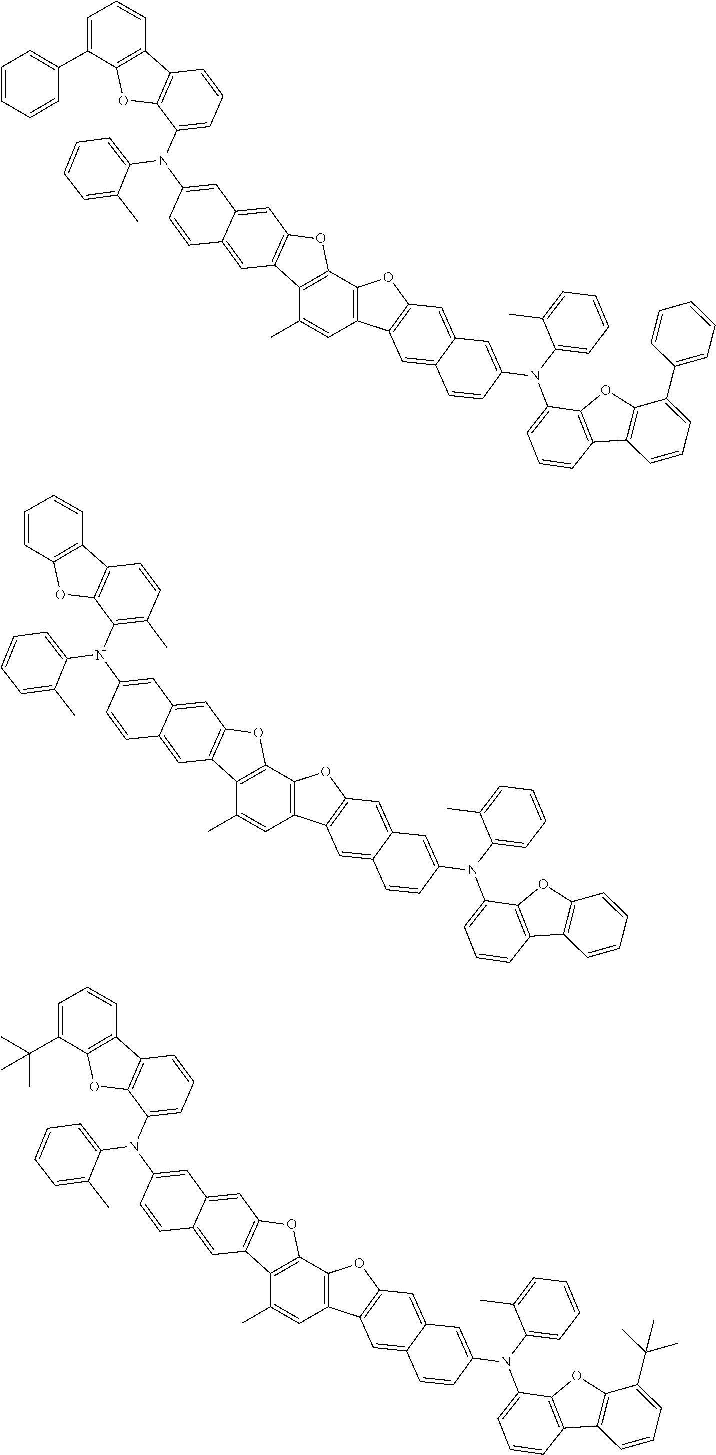 [SMILES: Cc1ccccc1N(c1ccc2cc3c(cc2c1)oc1c3cc(C)c2c3cc4ccc(N(c5ccccc5C)c5cccc6c5oc5c(-c7ccccc7)cccc56)cc4cc3oc12)c1cccc2c1oc1c(-c3ccccc3)cccc12 ; Cc1ccccc1N(c1ccc2cc3c(cc2c1)oc1c3cc(C)c2c3cc4ccc(N(c5ccccc5C)c5c(C)ccc6c5oc5ccccc56)cc4cc3oc12)c1cccc2c1oc1ccccc12 ; Cc1ccccc1N(c1ccc2cc3c(cc2c1)oc1c3cc(C)c2c3cc4ccc(N(c5ccccc5C)c5cccc6c5oc5c(C(C)(C)C)cccc56)cc4cc3oc12)c1cccc2c1oc1c(C(C)(C)C)cccc12]

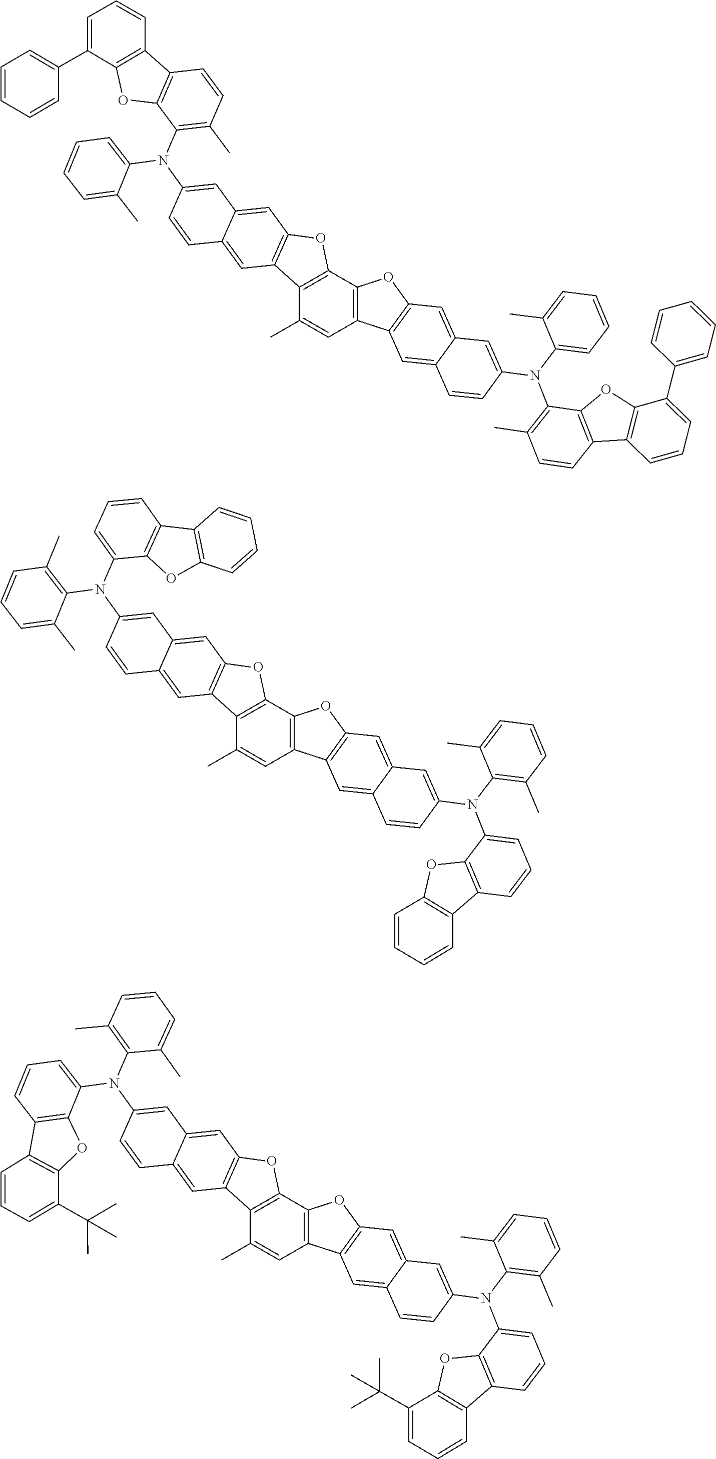 [SMILES: Cc1ccccc1N(c1ccc2cc3c(cc2c1)oc1c3cc(C)c2c3cc4ccc(N(c5ccccc5C)c5c(C)ccc6c5oc5c(-c7ccccc7)cccc56)cc4cc3oc12)c1c(C)ccc2c1oc1c(-c3ccccc3)cccc12 ; Cc1cccc(C)c1N(c1ccc2cc3c(cc2c1)oc1c3cc(C)c2c3cc4ccc(N(c5c(C)cccc5C)c5cccc6c5oc5ccccc56)cc4cc3oc12)c1cccc2c1oc1ccccc12 ; Cc1cccc(C)c1N(c1ccc2cc3c(cc2c1)oc1c3cc(C)c2c3cc4ccc(N(c5c(C)cccc5C)c5cccc6c5oc5c(C(C)(C)C)cccc56)cc4cc3oc12)c1cccc2c1oc1c(C(C)(C)C)cccc12]

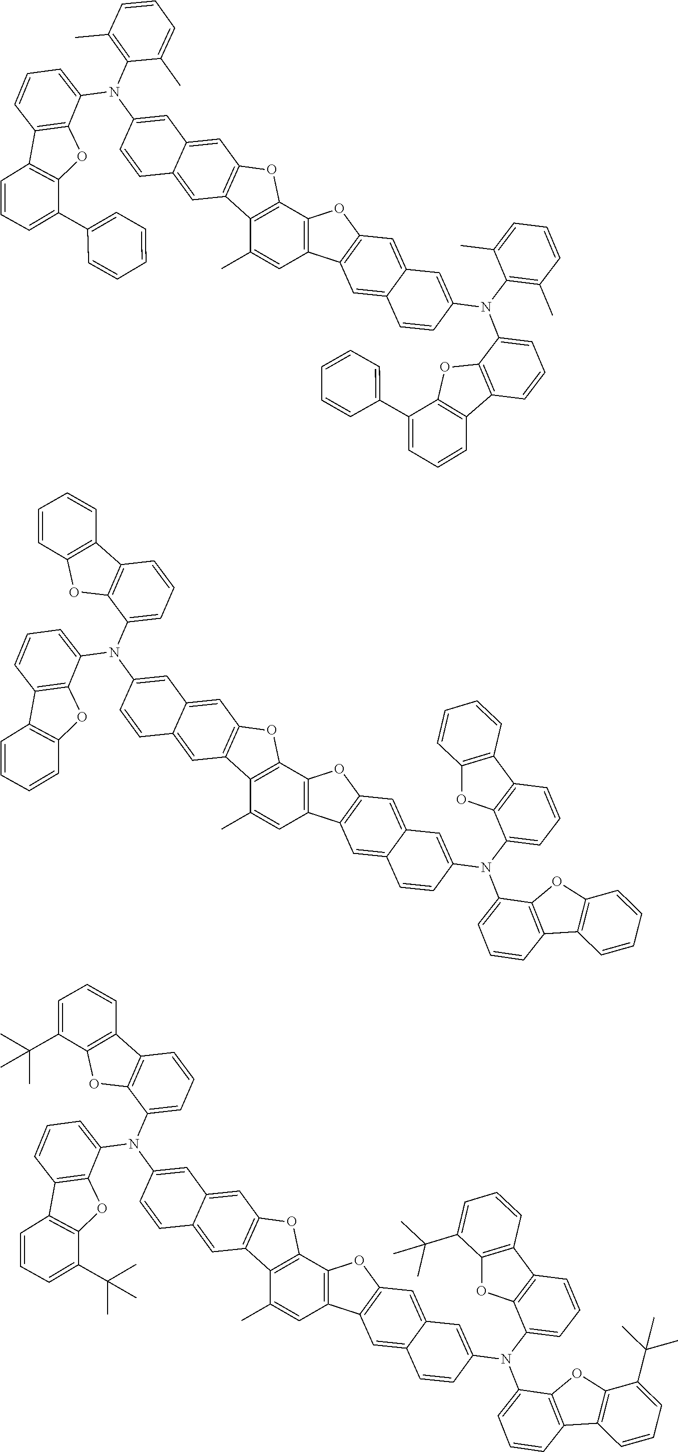 [SMILES: Cc1cccc(C)c1N(c1ccc2cc3c(cc2c1)oc1c3cc(C)c2c3cc4ccc(N(c5c(C)cccc5C)c5cccc6c5oc5c(-c7ccccc7)cccc56)cc4cc3oc12)c1cccc2c1oc1c(-c3ccccc3)cccc12 ; Cc1cc2c3cc4ccc(N(c5cccc6c5oc5ccccc56)c5cccc6c5oc5ccccc56)cc4cc3oc2c2oc3cc4cc(N(c5cccc6c5oc5ccccc56)c5cccc6c5oc5ccccc56)ccc4cc3c12 ; Cc1cc2c3cc4ccc(N(c5cccc6c5oc5c(C(C)(C)C)cccc56)c5cccc6c5oc5c(C(C)(C)C)cccc56)cc4cc3oc2c2oc3cc4cc(N(c5cccc6c5oc5c(C(C)(C)C)cccc56)c5cccc6c5oc5c(C(C)(C)C)cccc56)ccc4cc3c12]

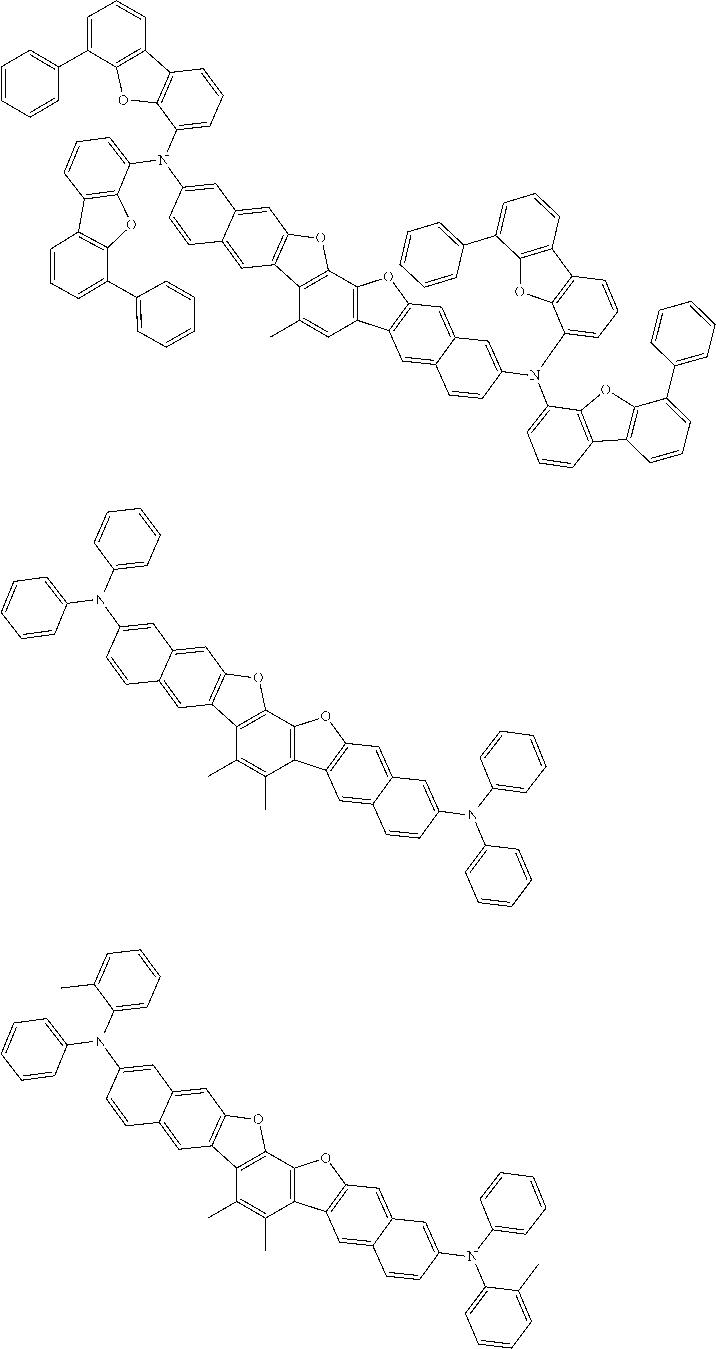 [SMILES: Cc1cc2c3cc4ccc(N(c5cccc6c5oc5c(-c7ccccc7)cccc56)c5cccc6c5oc5c(-c7ccccc7)cccc56)cc4cc3oc2c2oc3cc4cc(N(c5cccc6c5oc5c(-c7ccccc7)cccc56)c5cccc6c5oc5c(-c7ccccc7)cccc56)ccc4cc3c12 ; Cc1c(C)c2c3cc4ccc(N(c5ccccc5)c5ccccc5)cc4cc3oc2c2oc3cc4cc(N(c5ccccc5)c5ccccc5)ccc4cc3c12 ; Cc1ccccc1N(c1ccccc1)c1ccc2cc3c(cc2c1)oc1c2oc4cc5cc(N(c6ccccc6)c6ccccc6C)ccc5cc4c2c(C)c(C)c31]

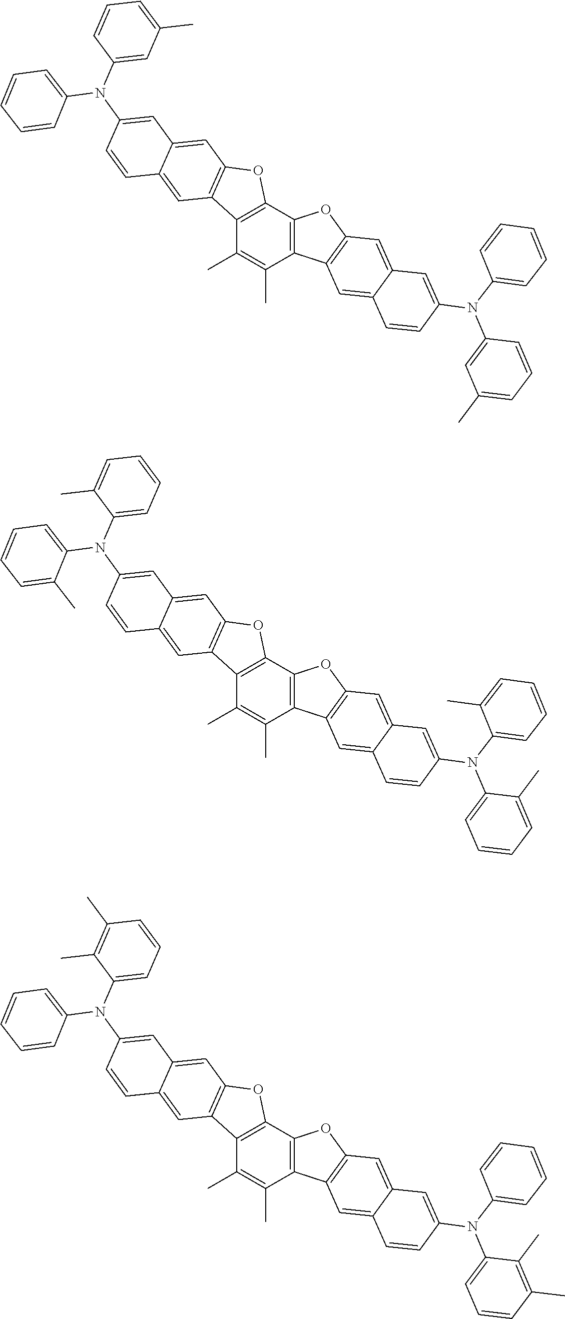 [SMILES: Cc1cccc(N(c2ccccc2)c2ccc3cc4c(cc3c2)oc2c3oc5cc6cc(N(c7ccccc7)c7cccc(C)c7)ccc6cc5c3c(C)c(C)c42)c1 ; Cc1ccccc1N(c1ccc2cc3c(cc2c1)oc1c2oc4cc5cc(N(c6ccccc6C)c6ccccc6C)ccc5cc4c2c(C)c(C)c31)c1ccccc1C ; Cc1cccc(N(c2ccccc2)c2ccc3cc4c(cc3c2)oc2c3oc5cc6cc(N(c7ccccc7)c7cccc(C)c7C)ccc6cc5c3c(C)c(C)c42)c1C]

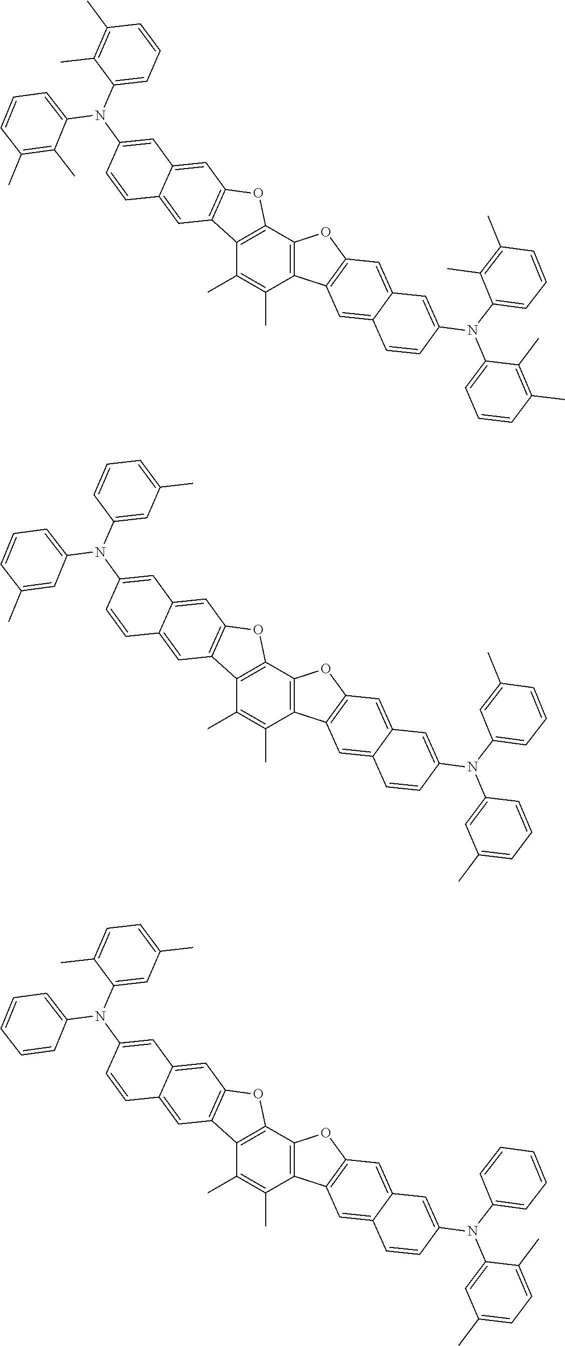 [SMILES: Cc1cccc(N(c2ccc3cc4c(cc3c2)oc2c3oc5cc6cc(N(c7cccc(C)c7C)c7cccc(C)c7C)ccc6cc5c3c(C)c(C)c42)c2cccc(C)c2C)c1C ; Cc1cccc(N(c2cccc(C)c2)c2ccc3cc4c(cc3c2)oc2c3oc5cc6cc(N(c7cccc(C)c7)c7cccc(C)c7)ccc6cc5c3c(C)c(C)c42)c1 ; Cc1ccc(C)c(N(c2ccccc2)c2ccc3cc4c(cc3c2)oc2c3oc5cc6cc(N(c7ccccc7)c7cc(C)ccc7C)ccc6cc5c3c(C)c(C)c42)c1]

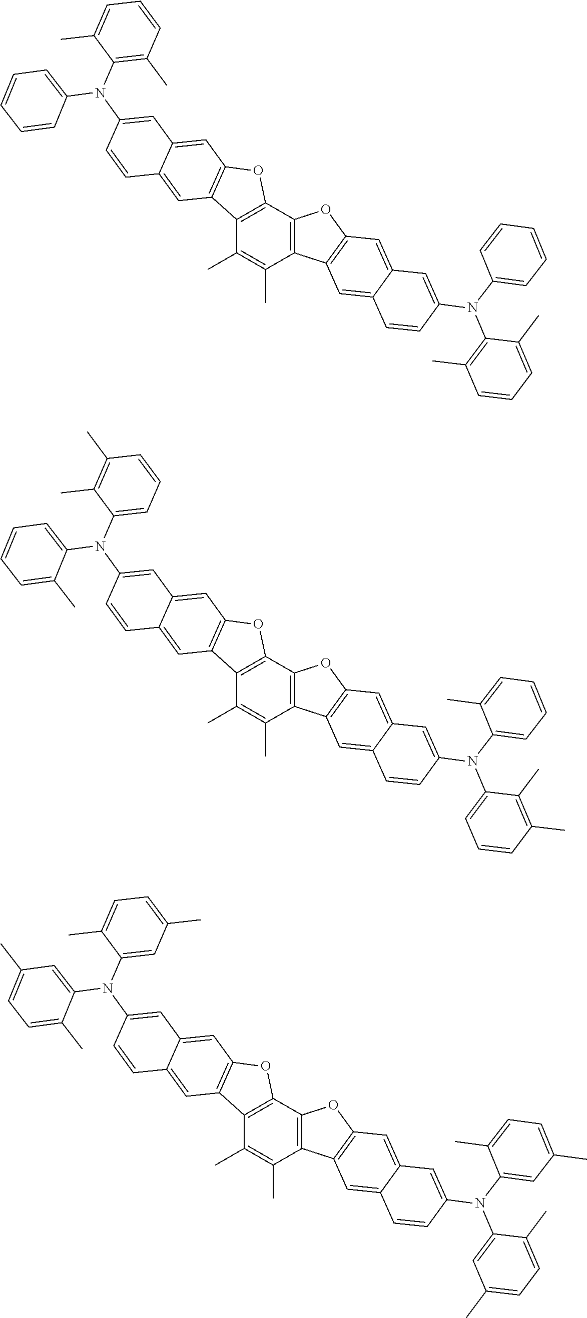

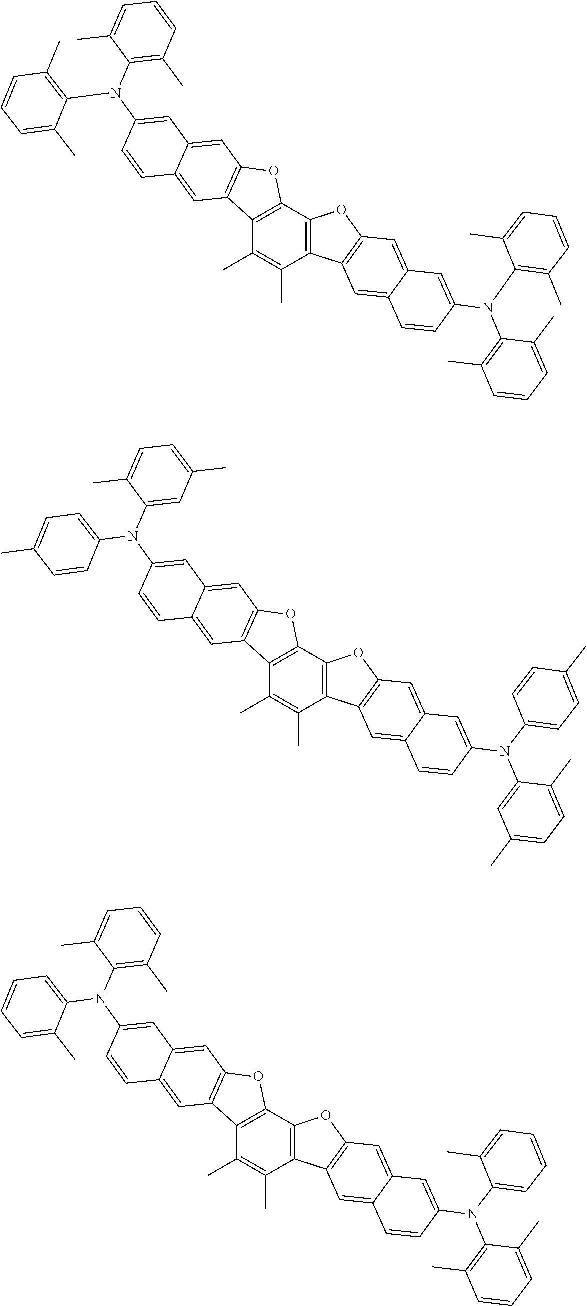

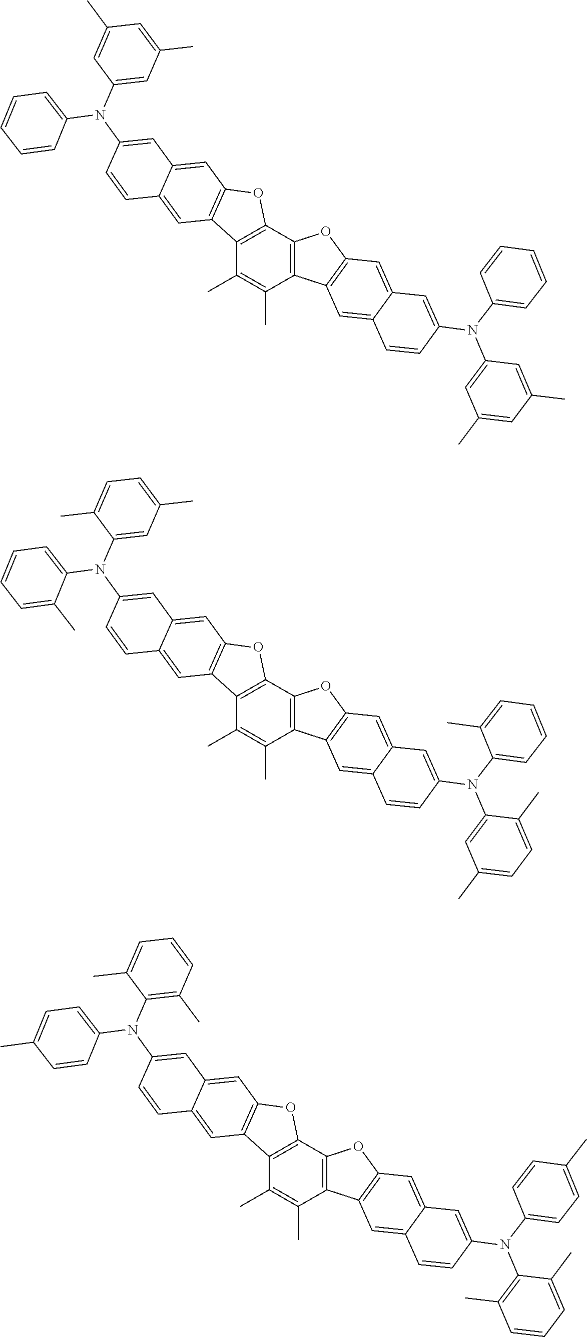

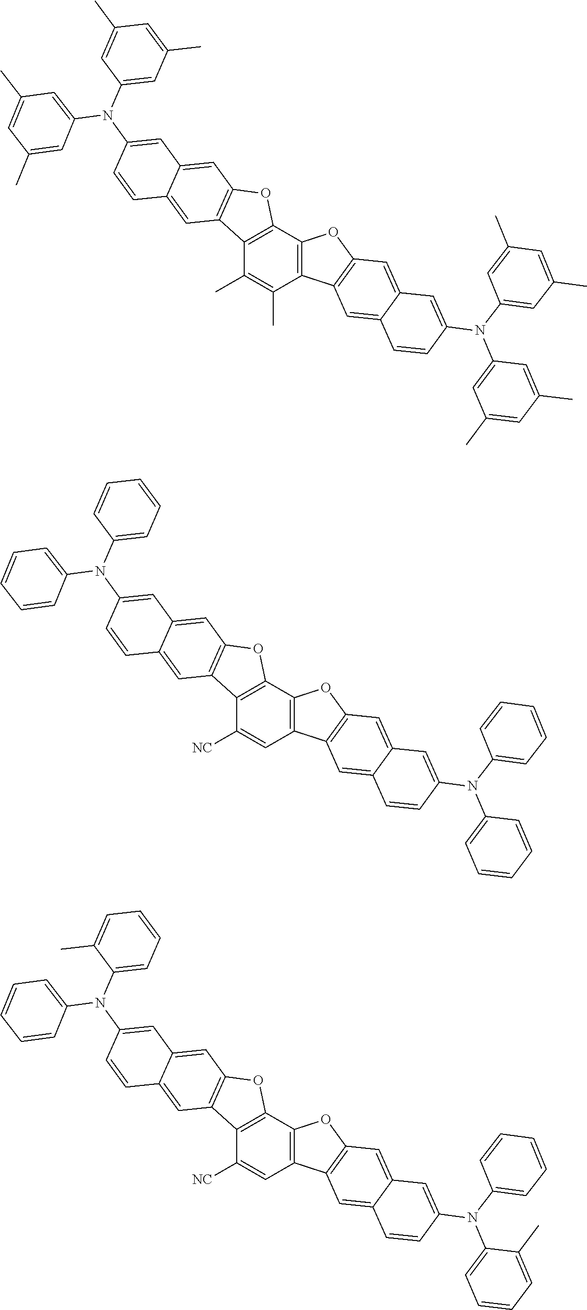

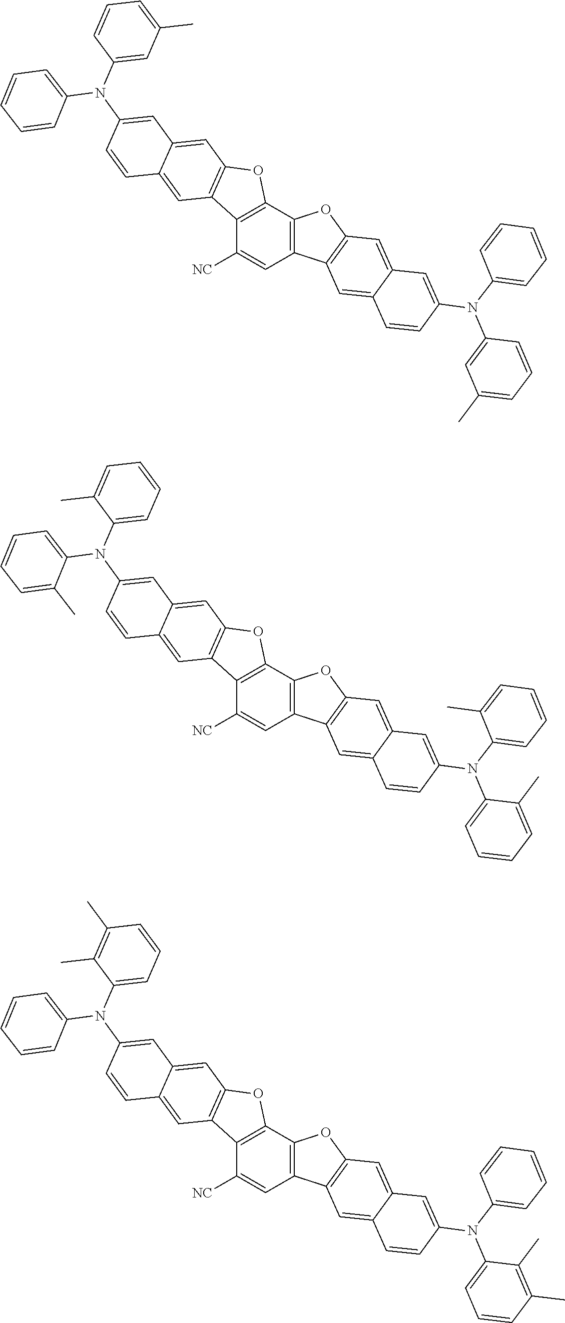 [SMILES: Cc1cccc(N(c2ccccc2)c2ccc3cc4c(cc3c2)oc2c4cc(C#N)c3c4cc5ccc(N(c6ccccc6)c6cccc(C)c6)cc5cc4oc23)c1 ; Cc1ccccc1N(c1ccc2cc3c(cc2c1)oc1c3cc(C#N)c2c3cc4ccc(N(c5ccccc5C)c5ccccc5C)cc4cc3oc12)c1ccccc1C ; Cc1cccc(N(c2ccccc2)c2ccc3cc4c(cc3c2)oc2c4cc(C#N)c3c4cc5ccc(N(c6ccccc6)c6cccc(C)c6C)cc5cc4oc23)c1C]

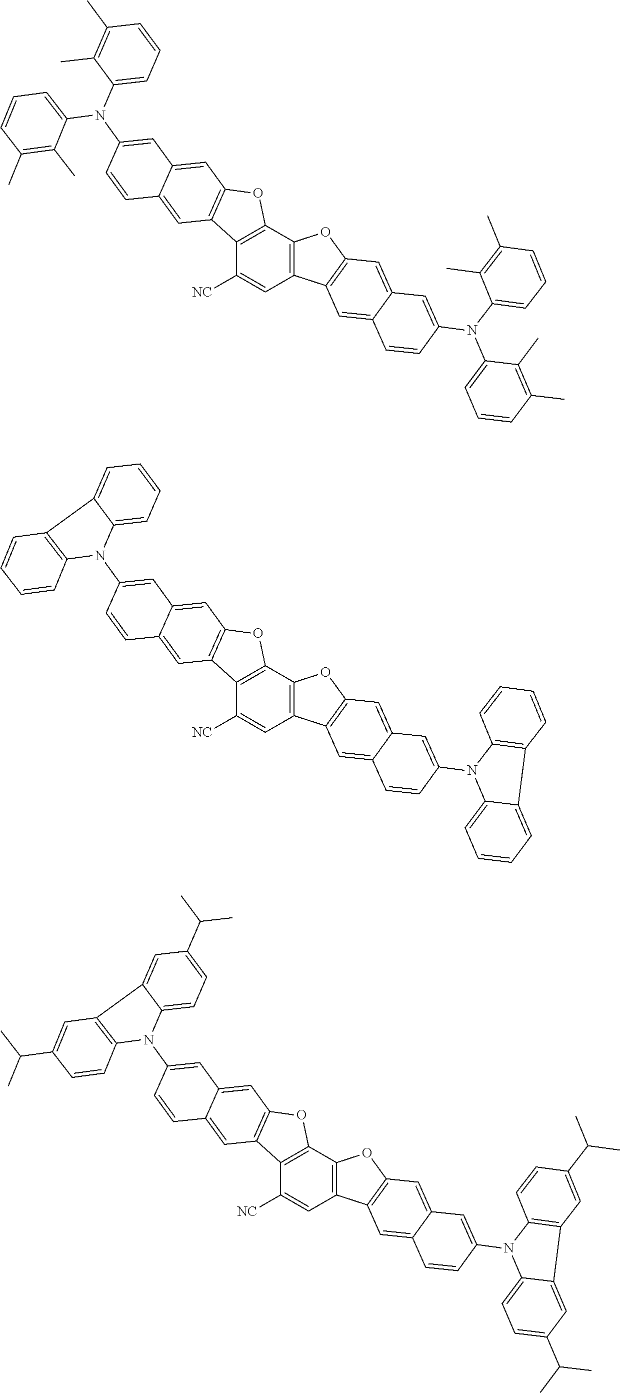

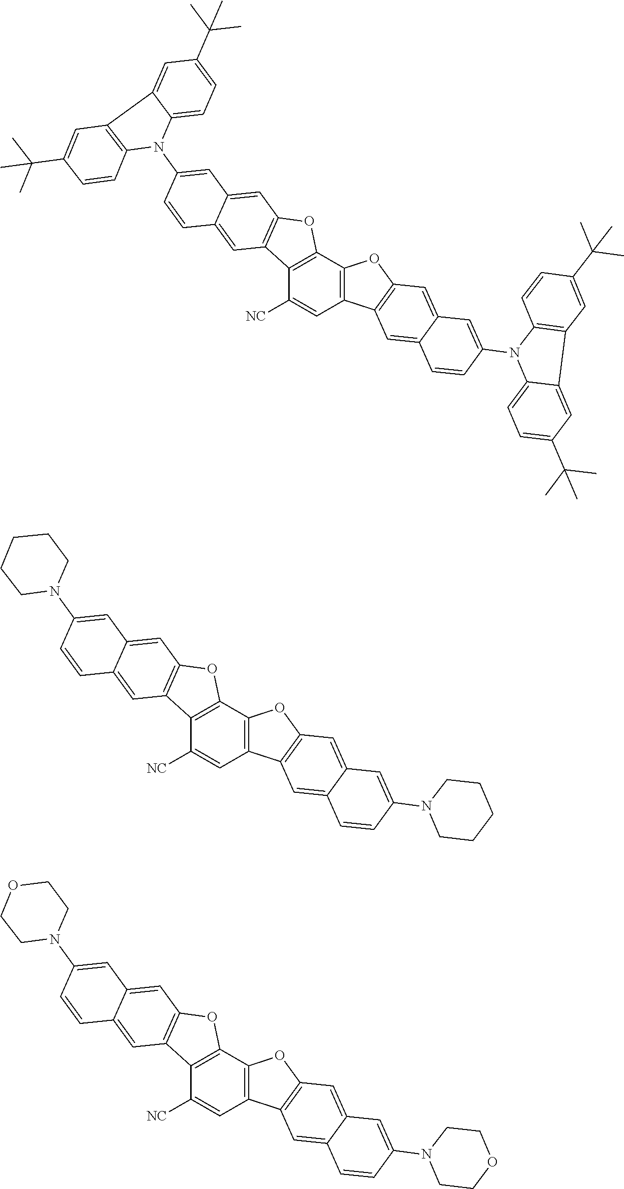 [SMILES: CC(C)(C)c1ccc2c(c1)c1cc(C(C)(C)C)ccc1n2-c1ccc2cc3c(cc2c1)oc1c3cc(C#N)c2c3cc4ccc(-n5c6ccc(C(C)(C)C)cc6c6cc(C(C)(C)C)ccc65)cc4cc3oc12 ; N#Cc1cc2c3cc4ccc(N5CCCCC5)cc4cc3oc2c2oc3cc4cc(N5CCCCC5)ccc4cc3c12 ; N#Cc1cc2c3cc4ccc(N5CCOCC5)cc4cc3oc2c2oc3cc4cc(N5CCOCC5)ccc4cc3c12]

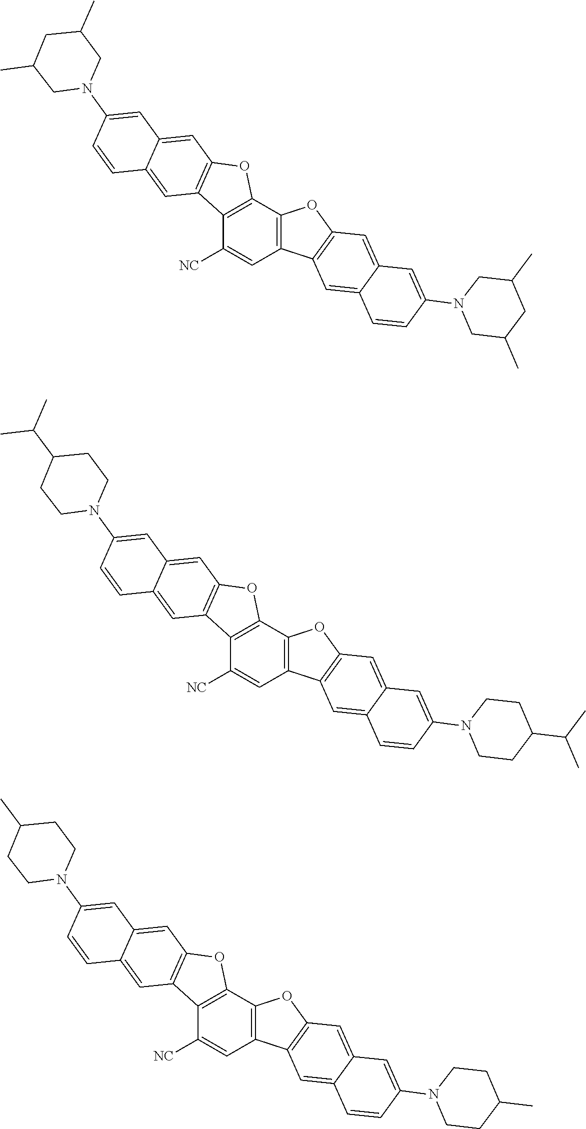

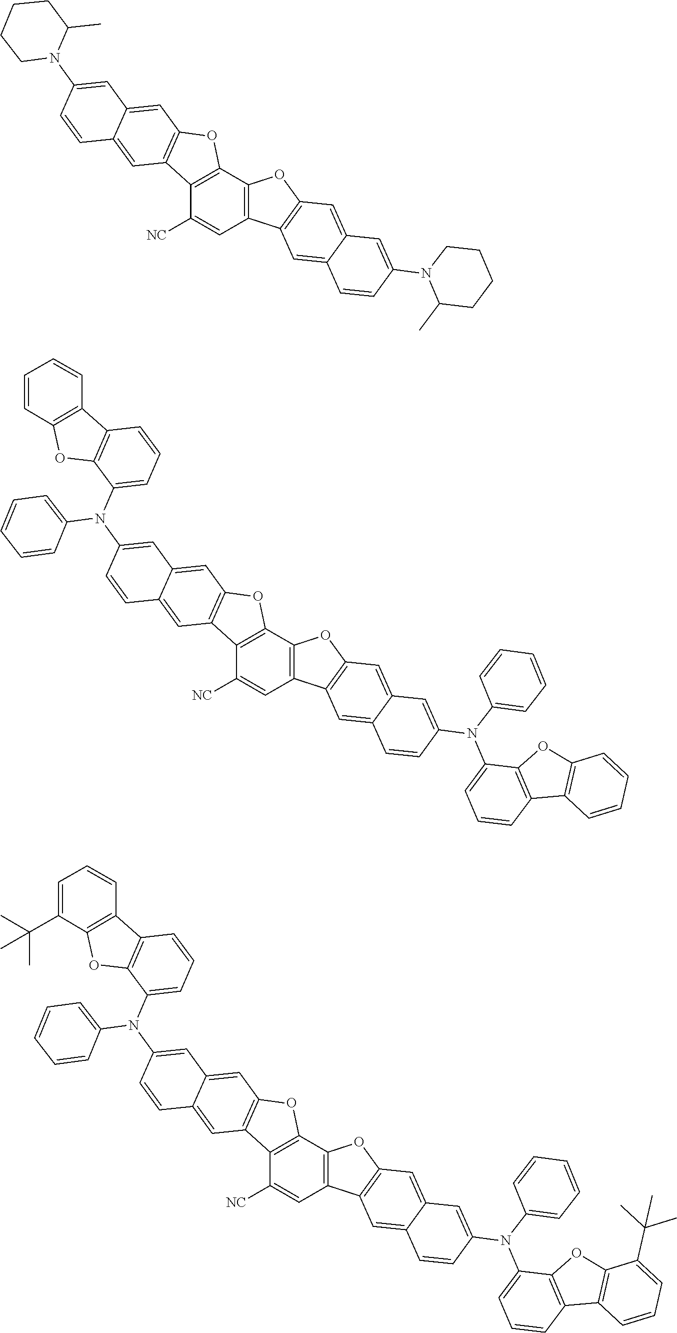 [SMILES: CC1CCCCN1c1ccc2cc3c(cc2c1)oc1c3cc(C#N)c2c3cc4ccc(N5CCCCC5C)cc4cc3oc12 ; N#Cc1cc2c3cc4ccc(N(c5ccccc5)c5cccc6c5oc5ccccc56)cc4cc3oc2c2oc3cc4cc(N(c5ccccc5)c5cccc6c5oc5ccccc56)ccc4cc3c12 ; CC(C)(C)c1cccc2c1oc1c(N(c3ccccc3)c3ccc4cc5c(cc4c3)oc3c5cc(C#N)c4c5cc6ccc(N(c7ccccc7)c7cccc8c7oc7c(C(C)(C)C)cccc78)cc6cc5oc34)cccc12]

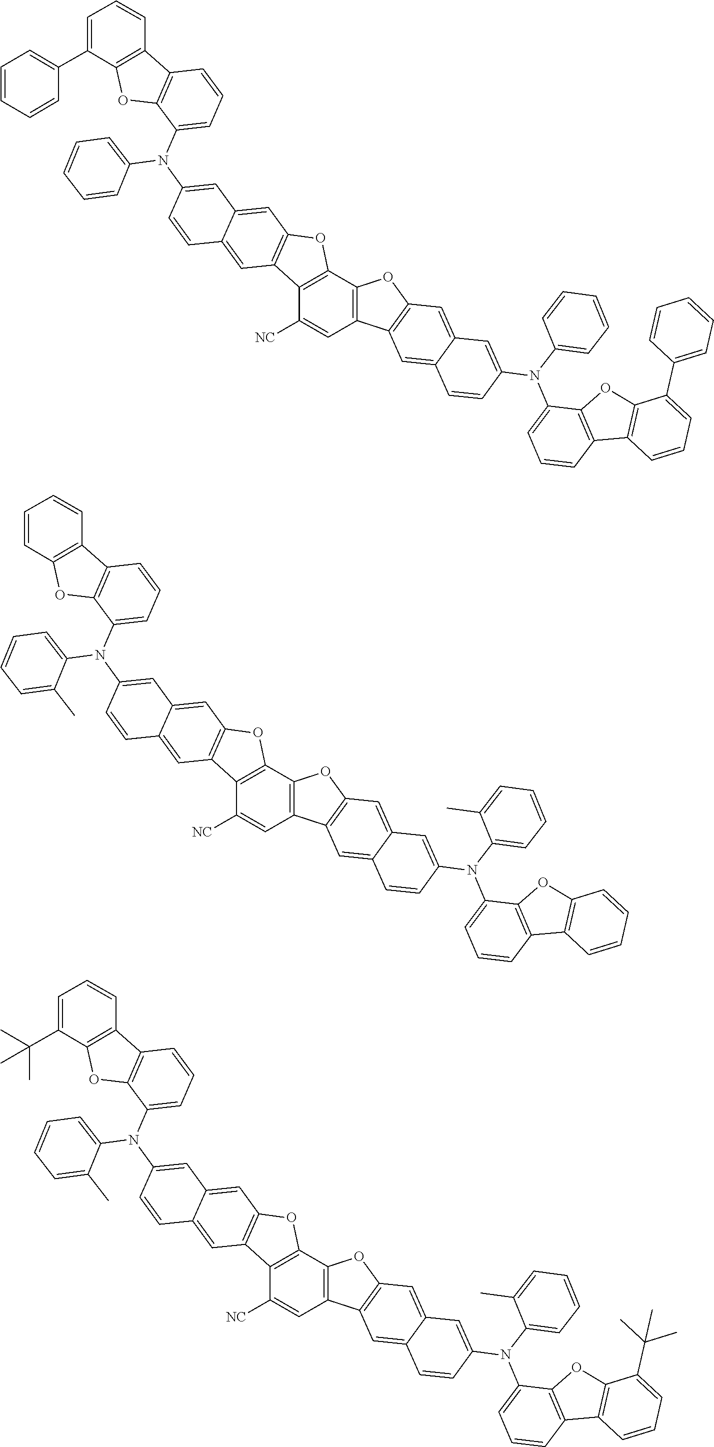 [SMILES: N#Cc1cc2c3cc4ccc(N(c5ccccc5)c5cccc6c5oc5c(-c7ccccc7)cccc56)cc4cc3oc2c2oc3cc4cc(N(c5ccccc5)c5cccc6c5oc5c(-c7ccccc7)cccc56)ccc4cc3c12 ; Cc1ccccc1N(c1ccc2cc3c(cc2c1)oc1c3cc(C#N)c2c3cc4ccc(N(c5ccccc5C)c5cccc6c5oc5ccccc56)cc4cc3oc12)c1cccc2c1oc1ccccc12 ; Cc1ccccc1N(c1ccc2cc3c(cc2c1)oc1c3cc(C#N)c2c3cc4ccc(N(c5ccccc5C)c5cccc6c5oc5c(C(C)(C)C)cccc56)cc4cc3oc12)c1cccc2c1oc1c(C(C)(C)C)cccc12]

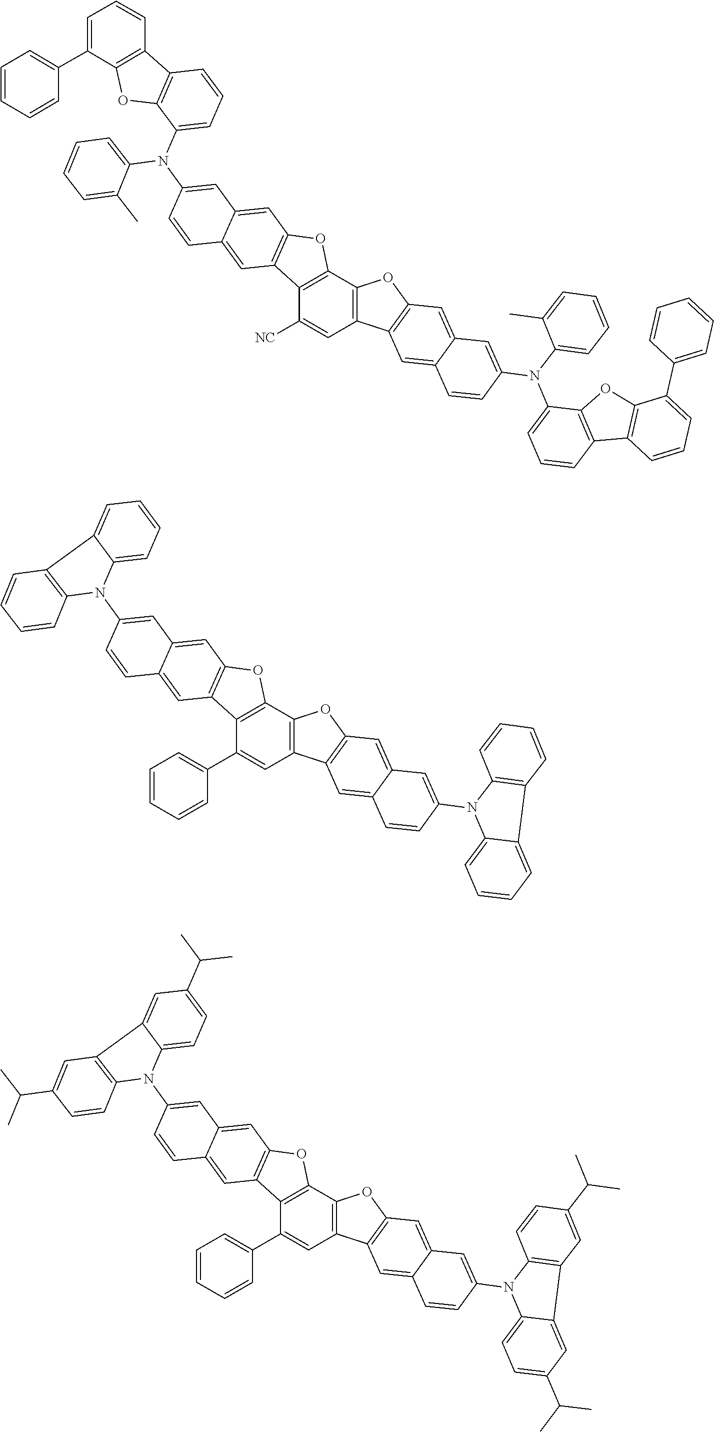 [SMILES: Cc1ccccc1N(c1ccc2cc3c(cc2c1)oc1c3cc(C#N)c2c3cc4ccc(N(c5ccccc5C)c5cccc6c5oc5c(-c7ccccc7)cccc56)cc4cc3oc12)c1cccc2c1oc1c(-c3ccccc3)cccc12 ; c1ccc(-c2cc3c4cc5ccc(-n6c7ccccc7c7ccccc76)cc5cc4oc3c3oc4cc5cc(-n6c7ccccc7c7ccccc76)ccc5cc4c23)cc1 ; CC(C)c1ccc2c(c1)c1cc(C(C)C)ccc1n2-c1ccc2cc3c(cc2c1)oc1c3cc(-c2ccccc2)c2c3cc4ccc(-n5c6ccc(C(C)C)cc6c6cc(C(C)C)ccc65)cc4cc3oc12]

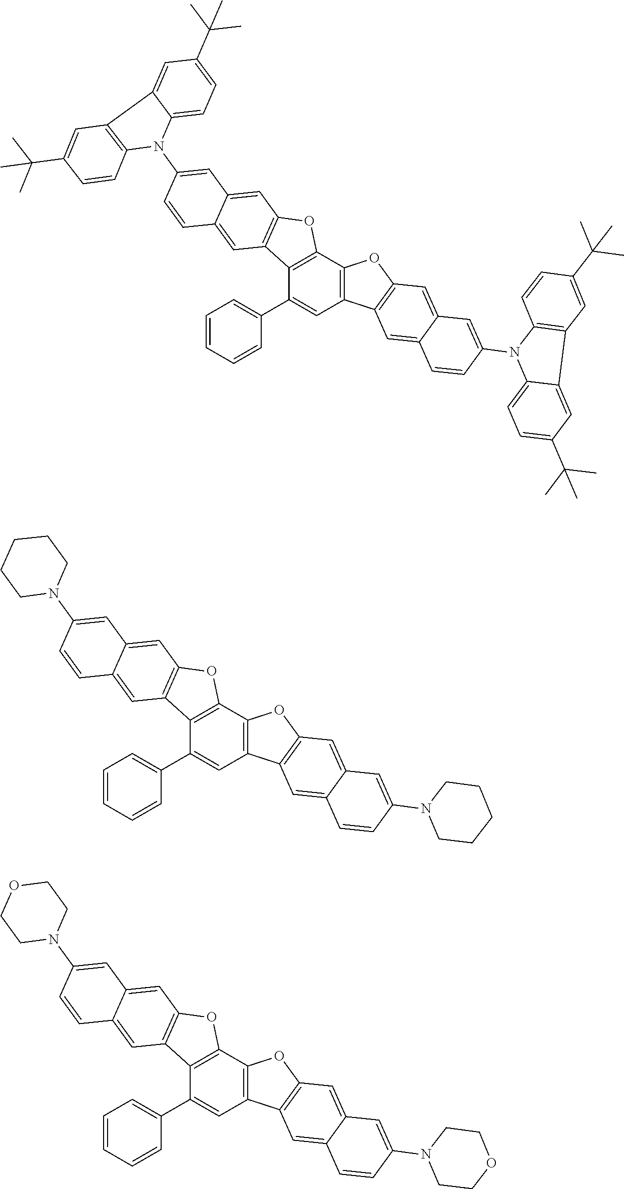 [SMILES: CC(C)(C)c1ccc2c(c1)c1cc(C(C)(C)C)ccc1n2-c1ccc2cc3c(cc2c1)oc1c3cc(-c2ccccc2)c2c3cc4ccc(-n5c6ccc(C(C)(C)C)cc6c6cc(C(C)(C)C)ccc65)cc4cc3oc12 ; c1ccc(-c2cc3c4cc5ccc(N6CCCCC6)cc5cc4oc3c3oc4cc5cc(N6CCCCC6)ccc5cc4c23)cc1 ; c1ccc(-c2cc3c4cc5ccc(N6CCOCC6)cc5cc4oc3c3oc4cc5cc(N6CCOCC6)ccc5cc4c23)cc1]

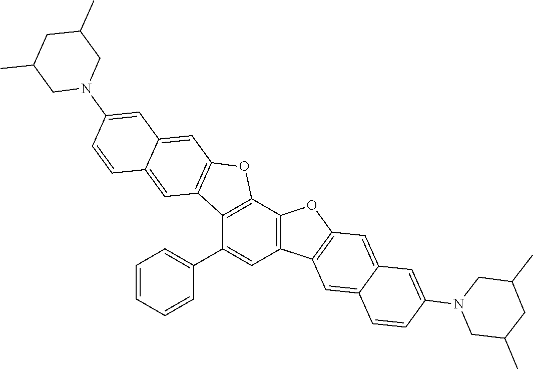

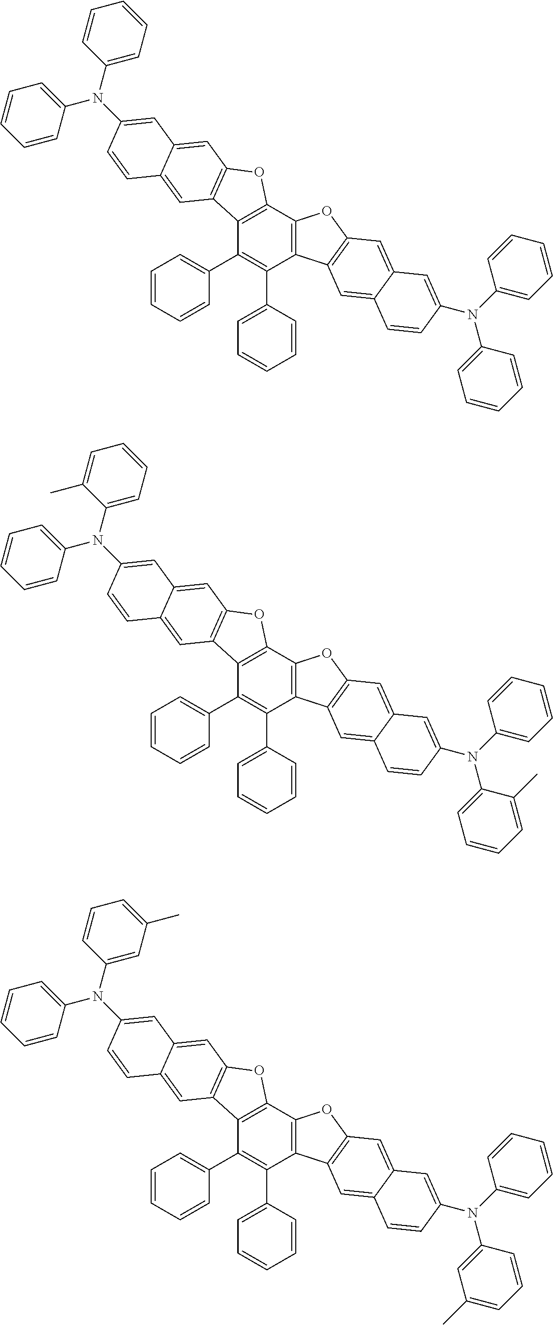 [SMILES: c1ccc(-c2c(-c3ccccc3)c3c4cc5ccc(N(c6ccccc6)c6ccccc6)cc5cc4oc3c3oc4cc5cc(N(c6ccccc6)c6ccccc6)ccc5cc4c23)cc1 ; Cc1ccccc1N(c1ccccc1)c1ccc2cc3c(cc2c1)oc1c2oc4cc5cc(N(c6ccccc6)c6ccccc6C)ccc5cc4c2c(-c2ccccc2)c(-c2ccccc2)c31 ; Cc1cccc(N(c2ccccc2)c2ccc3cc4c(cc3c2)oc2c3oc5cc6cc(N(c7ccccc7)c7cccc(C)c7)ccc6cc5c3c(-c3ccccc3)c(-c3ccccc3)c42)c1]

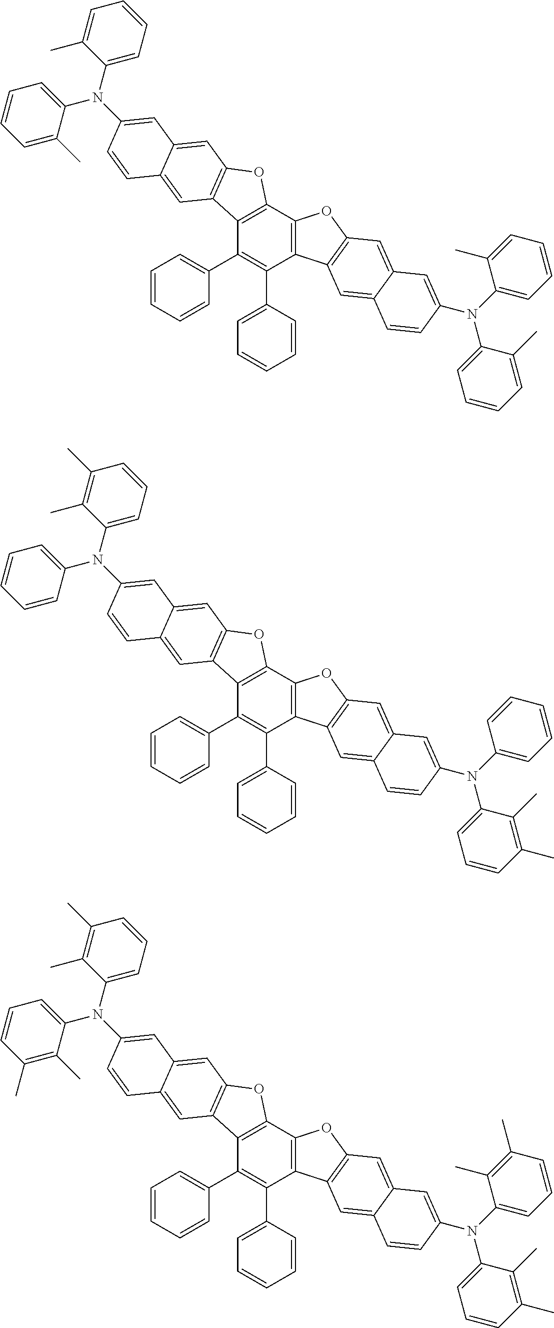 [SMILES: Cc1ccccc1N(c1ccc2cc3c(cc2c1)oc1c2oc4cc5cc(N(c6ccccc6C)c6ccccc6C)ccc5cc4c2c(-c2ccccc2)c(-c2ccccc2)c31)c1ccccc1C ; Cc1cccc(N(c2ccccc2)c2ccc3cc4c(cc3c2)oc2c3oc5cc6cc(N(c7ccccc7)c7cccc(C)c7C)ccc6cc5c3c(-c3ccccc3)c(-c3ccccc3)c42)c1C ; Cc1cccc(N(c2ccc3cc4c(cc3c2)oc2c3oc5cc6cc(N(c7cccc(C)c7C)c7cccc(C)c7C)ccc6cc5c3c(-c3ccccc3)c(-c3ccccc3)c42)c2cccc(C)c2C)c1C]

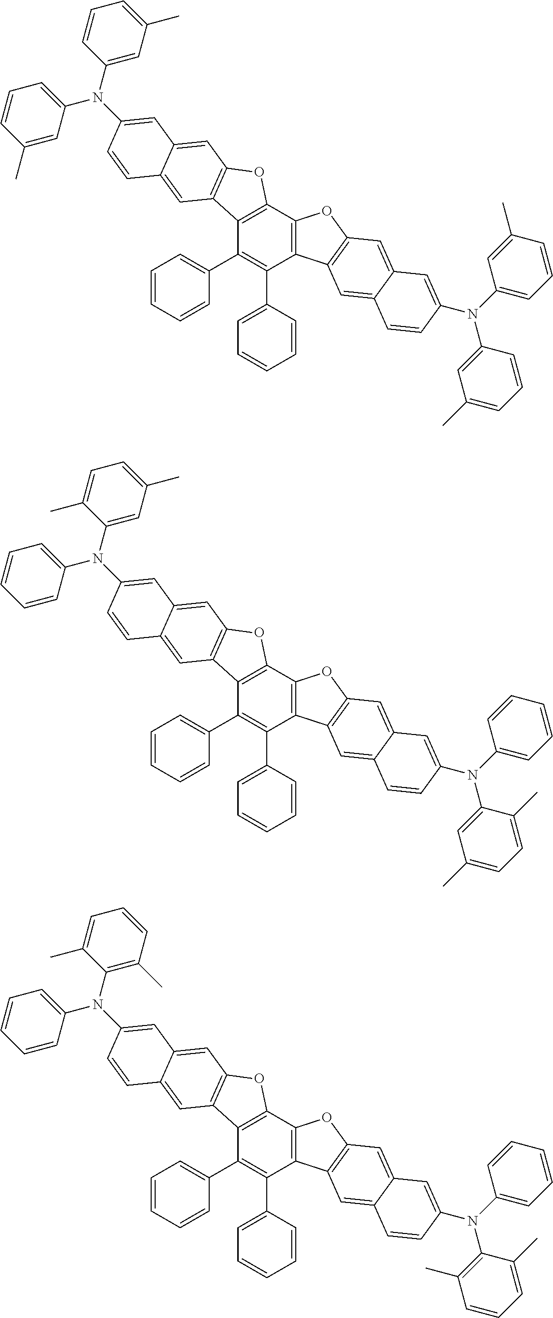 [SMILES: Cc1cccc(N(c2cccc(C)c2)c2ccc3cc4c(cc3c2)oc2c3oc5cc6cc(N(c7cccc(C)c7)c7cccc(C)c7)ccc6cc5c3c(-c3ccccc3)c(-c3ccccc3)c42)c1 ; Cc1ccc(C)c(N(c2ccccc2)c2ccc3cc4c(cc3c2)oc2c3oc5cc6cc(N(c7ccccc7)c7cc(C)ccc7C)ccc6cc5c3c(-c3ccccc3)c(-c3ccccc3)c42)c1 ; Cc1cccc(C)c1N(c1ccccc1)c1ccc2cc3c(cc2c1)oc1c2oc4cc5cc(N(c6ccccc6)c6c(C)cccc6C)ccc5cc4c2c(-c2ccccc2)c(-c2ccccc2)c31]

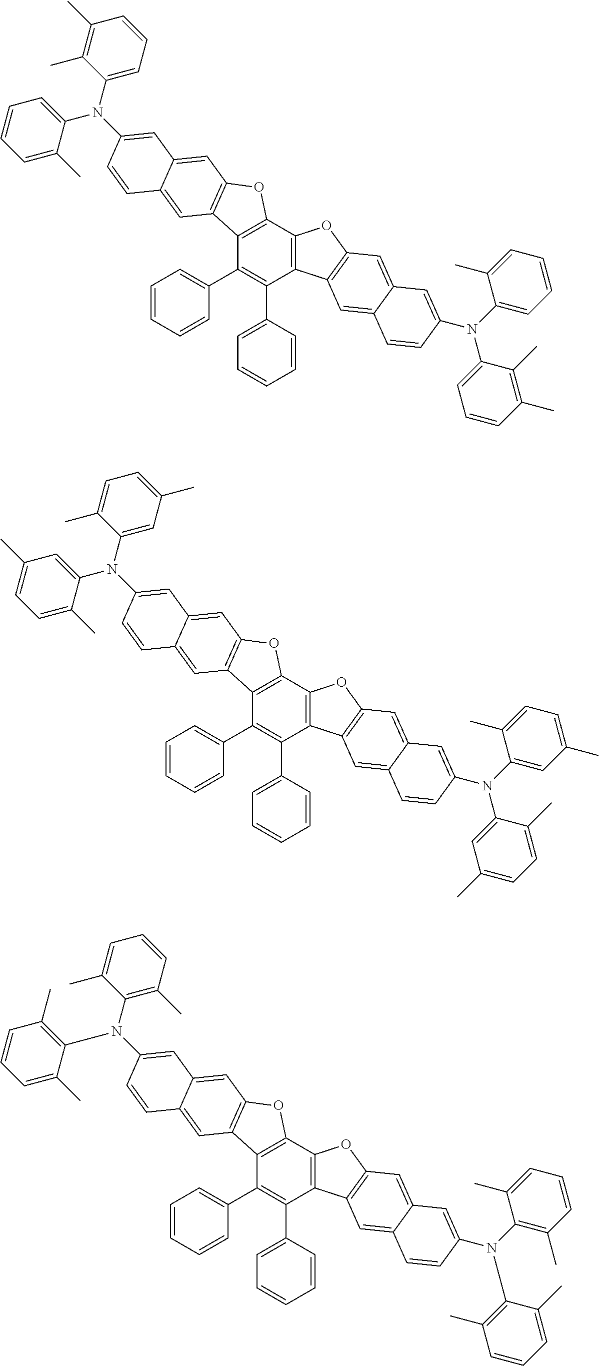

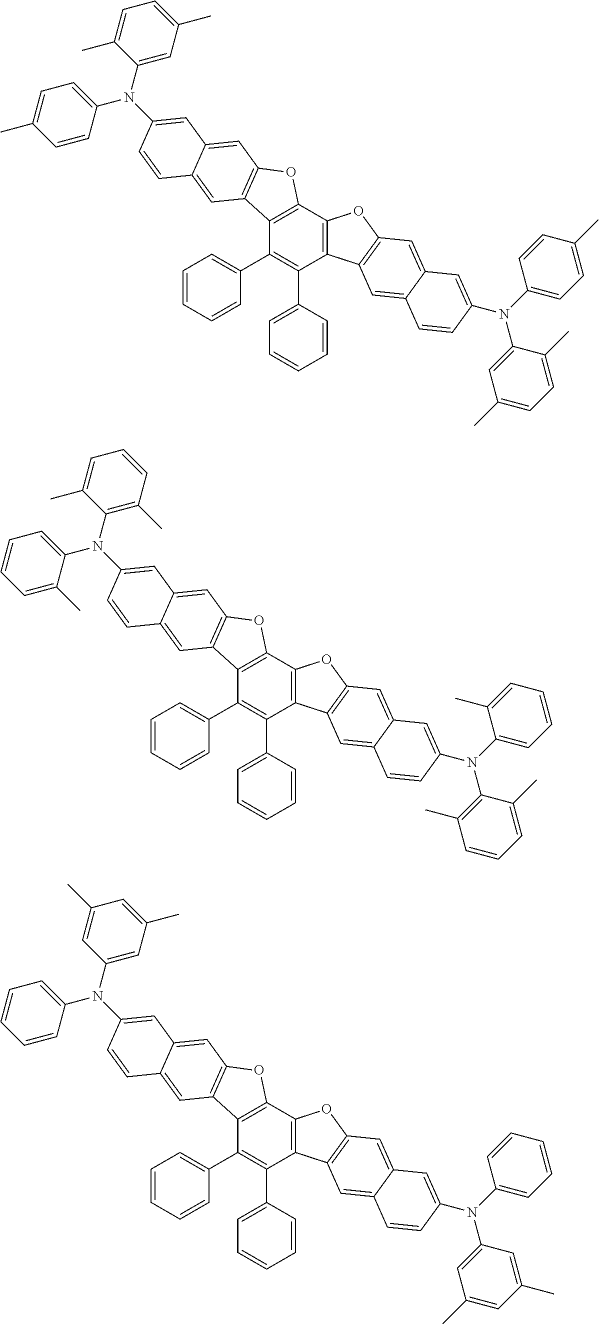

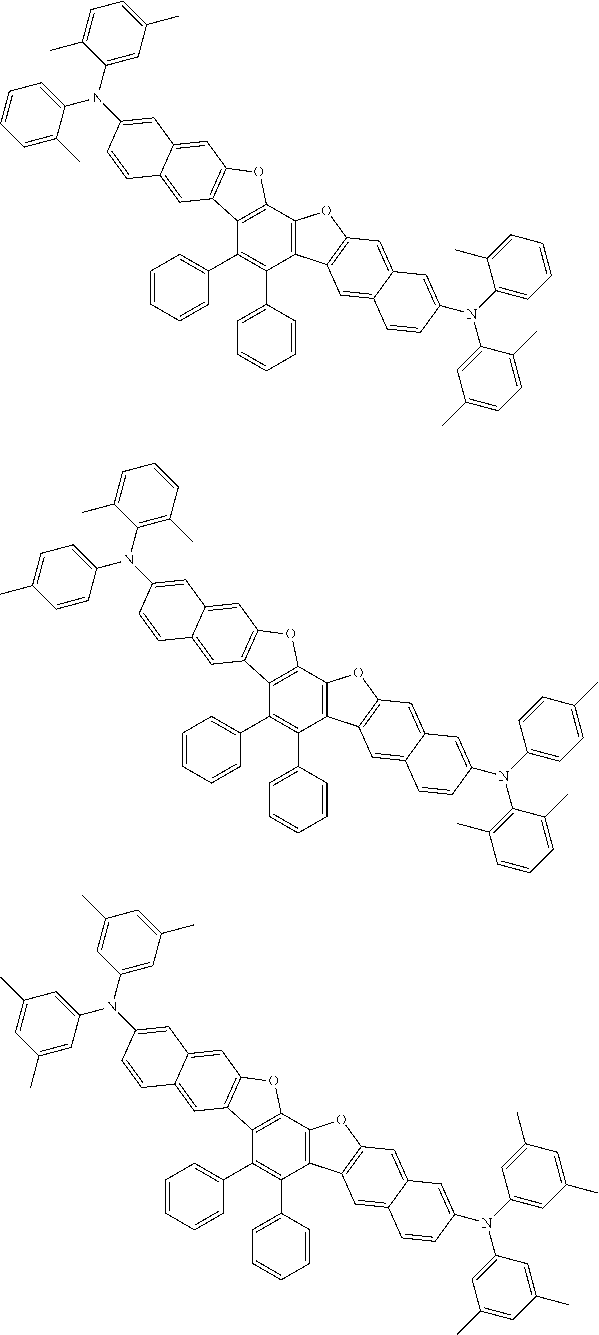

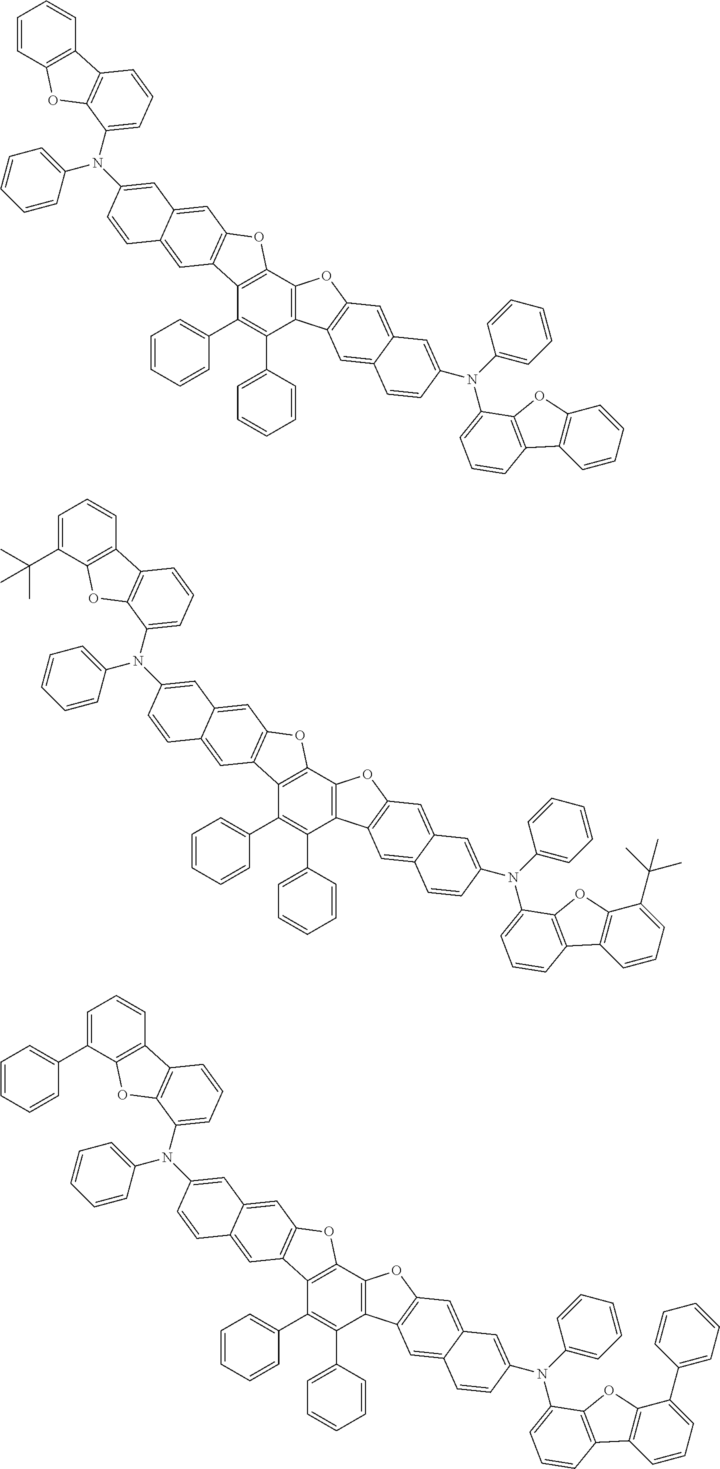 [SMILES: c1ccc(-c2c(-c3ccccc3)c3c4cc5ccc(N(c6ccccc6)c6cccc7c6oc6ccccc67)cc5cc4oc3c3oc4cc5cc(N(c6ccccc6)c6cccc7c6oc6ccccc67)ccc5cc4c23)cc1 ; CC(C)(C)c1cccc2c1oc1c(N(c3ccccc3)c3ccc4cc5c(cc4c3)oc3c4oc6cc7cc(N(c8ccccc8)c8cccc9c8oc8c(C(C)(C)C)cccc89)ccc7cc6c4c(-c4ccccc4)c(-c4ccccc4)c53)cccc12 ; c1ccc(-c2cccc3c2oc2c(N(c4ccccc4)c4ccc5cc6c(cc5c4)oc4c5oc7cc8cc(N(c9ccccc9)c9cccc%10c9oc9c(-c%11ccccc%11)cccc9%10)ccc8cc7c5c(-c5ccccc5)c(-c5ccccc5)c64)cccc23)cc1]

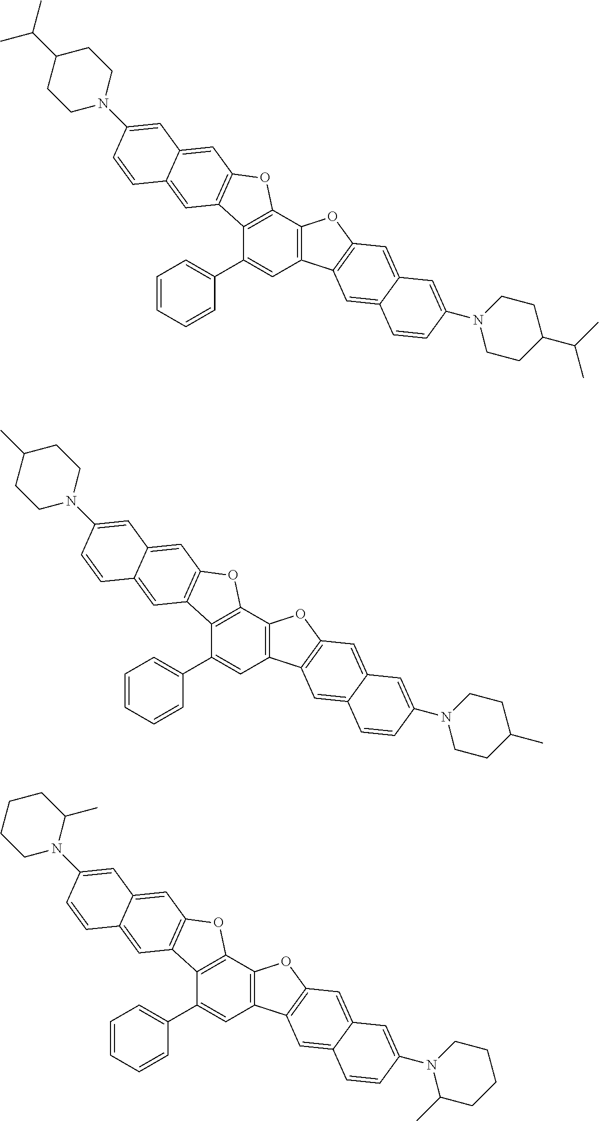

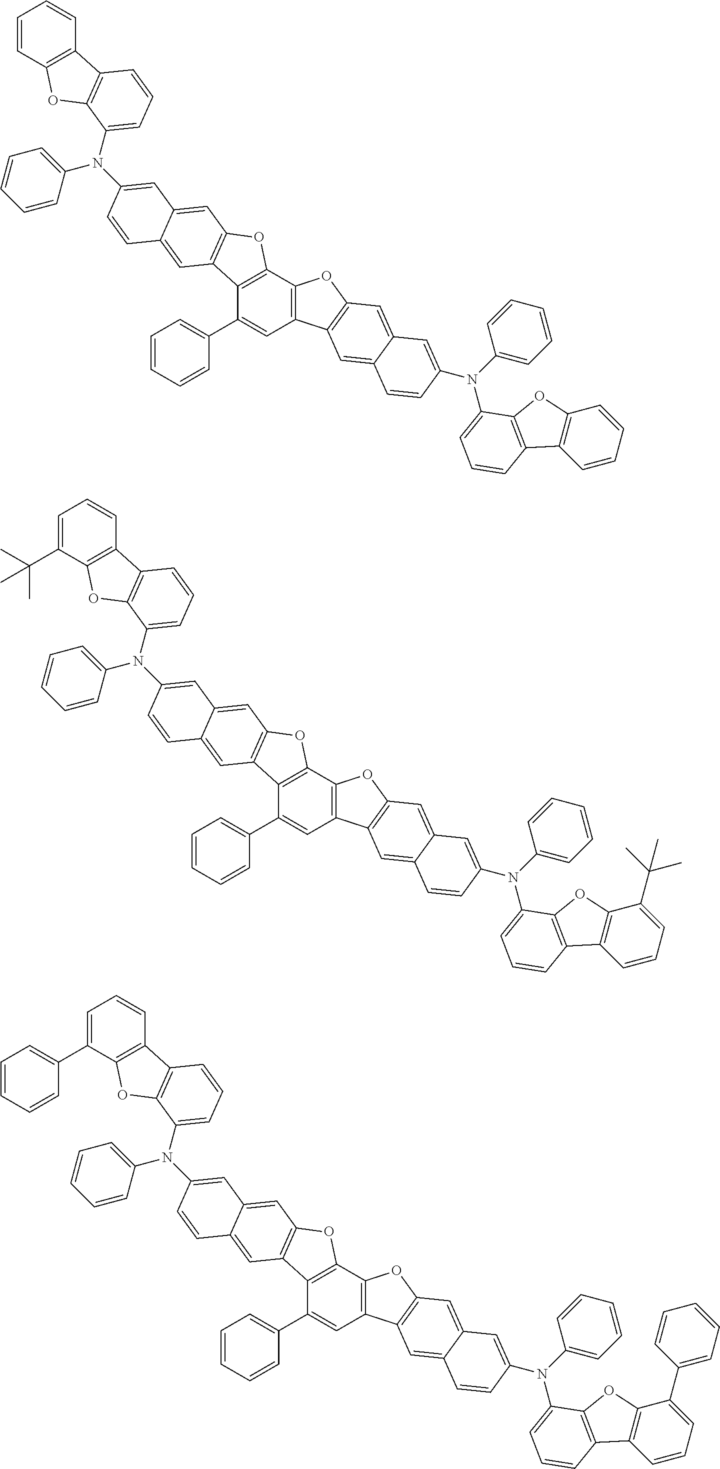 [SMILES: c1ccc(-c2cc3c4cc5ccc(N(c6ccccc6)c6cccc7c6oc6ccccc67)cc5cc4oc3c3oc4cc5cc(N(c6ccccc6)c6cccc7c6oc6ccccc67)ccc5cc4c23)cc1 ; CC(C)(C)c1cccc2c1oc1c(N(c3ccccc3)c3ccc4cc5c(cc4c3)oc3c5cc(-c4ccccc4)c4c5cc6ccc(N(c7ccccc7)c7cccc8c7oc7c(C(C)(C)C)cccc78)cc6cc5oc34)cccc12 ; c1ccc(-c2cccc3c2oc2c(N(c4ccccc4)c4ccc5cc6c(cc5c4)oc4c6cc(-c5ccccc5)c5c6cc7ccc(N(c8ccccc8)c8cccc9c8oc8c(-c%10ccccc%10)cccc89)cc7cc6oc45)cccc23)cc1]

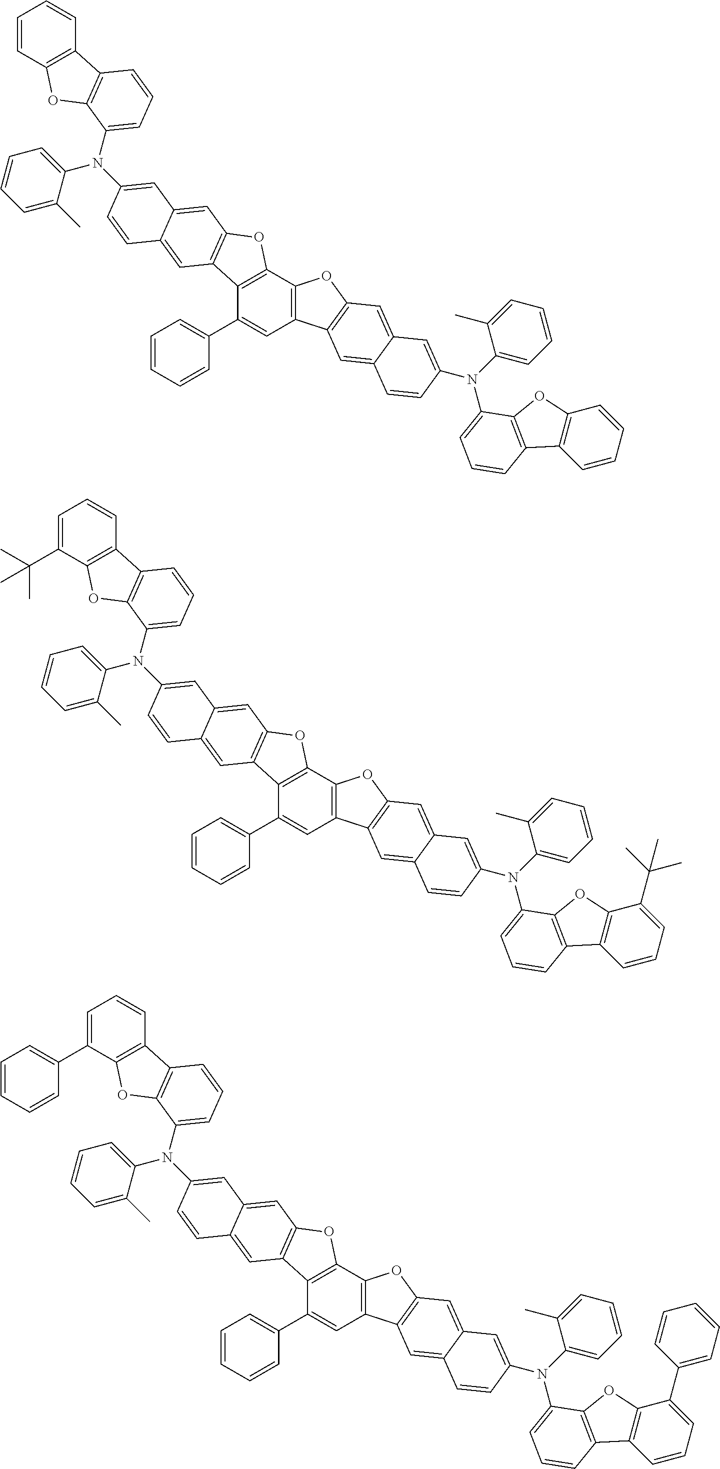 [SMILES: Cc1ccccc1N(c1ccc2cc3c(cc2c1)oc1c3cc(-c2ccccc2)c2c3cc4ccc(N(c5ccccc5C)c5cccc6c5oc5ccccc56)cc4cc3oc12)c1cccc2c1oc1ccccc12 ; Cc1ccccc1N(c1ccc2cc3c(cc2c1)oc1c3cc(-c2ccccc2)c2c3cc4ccc(N(c5ccccc5C)c5cccc6c5oc5c(C(C)(C)C)cccc56)cc4cc3oc12)c1cccc2c1oc1c(C(C)(C)C)cccc12 ; Cc1ccccc1N(c1ccc2cc3c(cc2c1)oc1c3cc(-c2ccccc2)c2c3cc4ccc(N(c5ccccc5C)c5cccc6c5oc5c(-c7ccccc7)cccc56)cc4cc3oc12)c1cccc2c1oc1c(-c3ccccc3)cccc12]

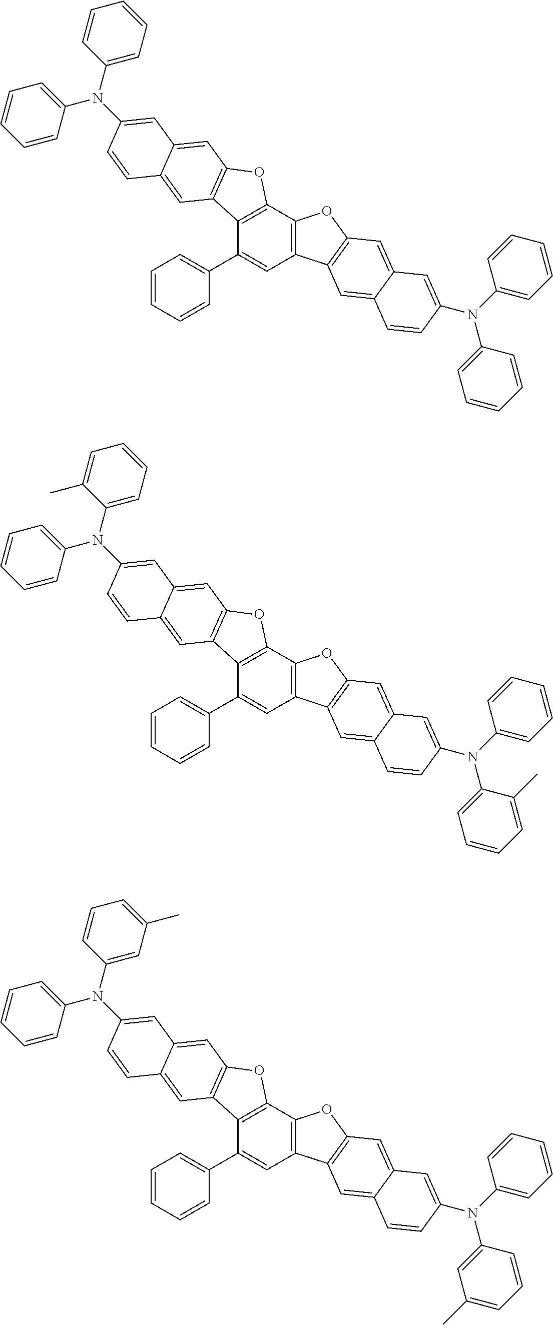 [SMILES: c1ccc(-c2cc3c4cc5ccc(N(c6ccccc6)c6ccccc6)cc5cc4oc3c3oc4cc5cc(N(c6ccccc6)c6ccccc6)ccc5cc4c23)cc1 ; Cc1ccccc1N(c1ccccc1)c1ccc2cc3c(cc2c1)oc1c3cc(-c2ccccc2)c2c3cc4ccc(N(c5ccccc5)c5ccccc5C)cc4cc3oc12 ; Cc1cccc(N(c2ccccc2)c2ccc3cc4c(cc3c2)oc2c4cc(-c3ccccc3)c3c4cc5ccc(N(c6ccccc6)c6cccc(C)c6)cc5cc4oc23)c1]

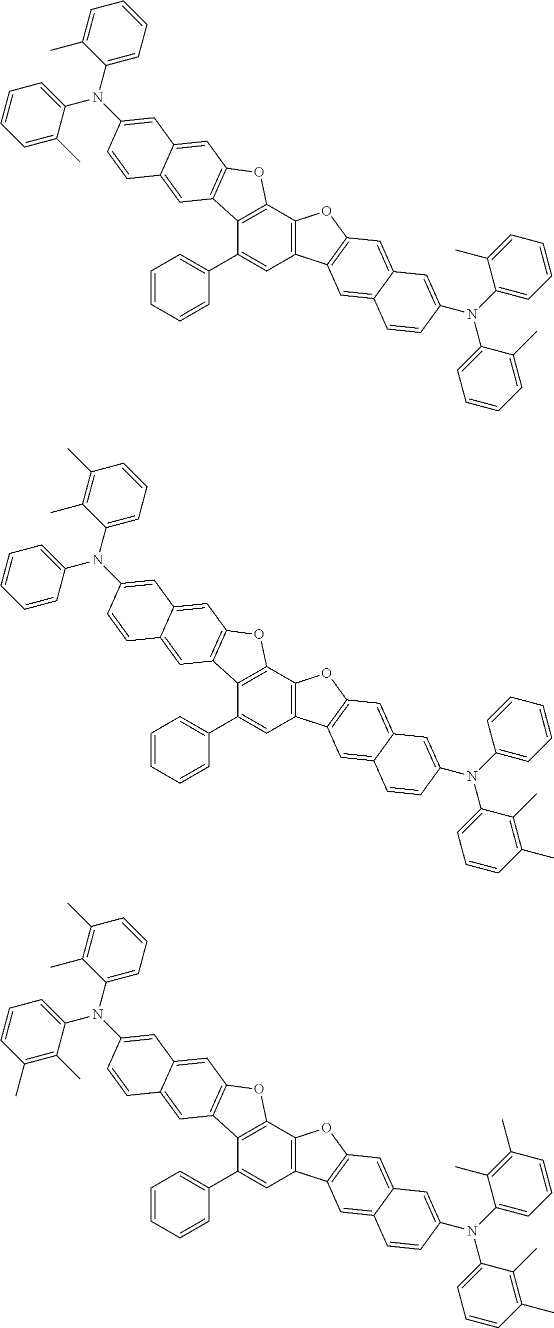 [SMILES: Cc1ccccc1N(c1ccc2cc3c(cc2c1)oc1c3cc(-c2ccccc2)c2c3cc4ccc(N(c5ccccc5C)c5ccccc5C)cc4cc3oc12)c1ccccc1C ; Cc1cccc(N(c2ccccc2)c2ccc3cc4c(cc3c2)oc2c4cc(-c3ccccc3)c3c4cc5ccc(N(c6ccccc6)c6cccc(C)c6C)cc5cc4oc23)c1C ; Cc1cccc(N(c2ccc3cc4c(cc3c2)oc2c4cc(-c3ccccc3)c3c4cc5ccc(N(c6cccc(C)c6C)c6cccc(C)c6C)cc5cc4oc23)c2cccc(C)c2C)c1C]

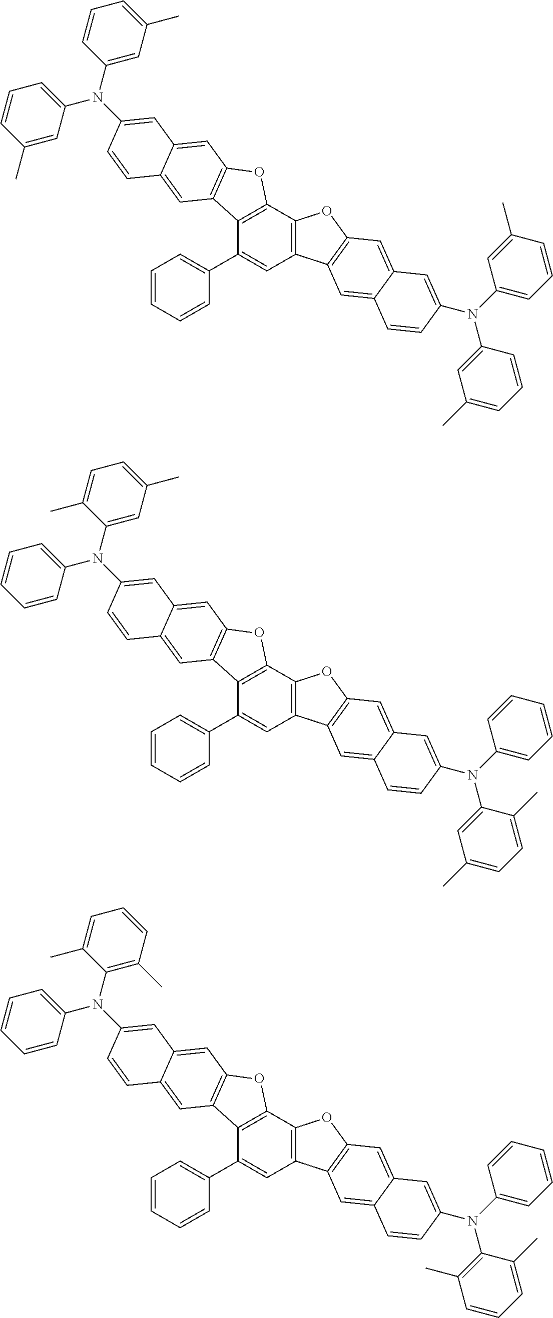 [SMILES: Cc1cccc(N(c2cccc(C)c2)c2ccc3cc4c(cc3c2)oc2c4cc(-c3ccccc3)c3c4cc5ccc(N(c6cccc(C)c6)c6cccc(C)c6)cc5cc4oc23)c1 ; Cc1ccc(C)c(N(c2ccccc2)c2ccc3cc4c(cc3c2)oc2c4cc(-c3ccccc3)c3c4cc5ccc(N(c6ccccc6)c6cc(C)ccc6C)cc5cc4oc23)c1 ; Cc1cccc(C)c1N(c1ccccc1)c1ccc2cc3c(cc2c1)oc1c3cc(-c2ccccc2)c2c3cc4ccc(N(c5ccccc5)c5c(C)cccc5C)cc4cc3oc12]

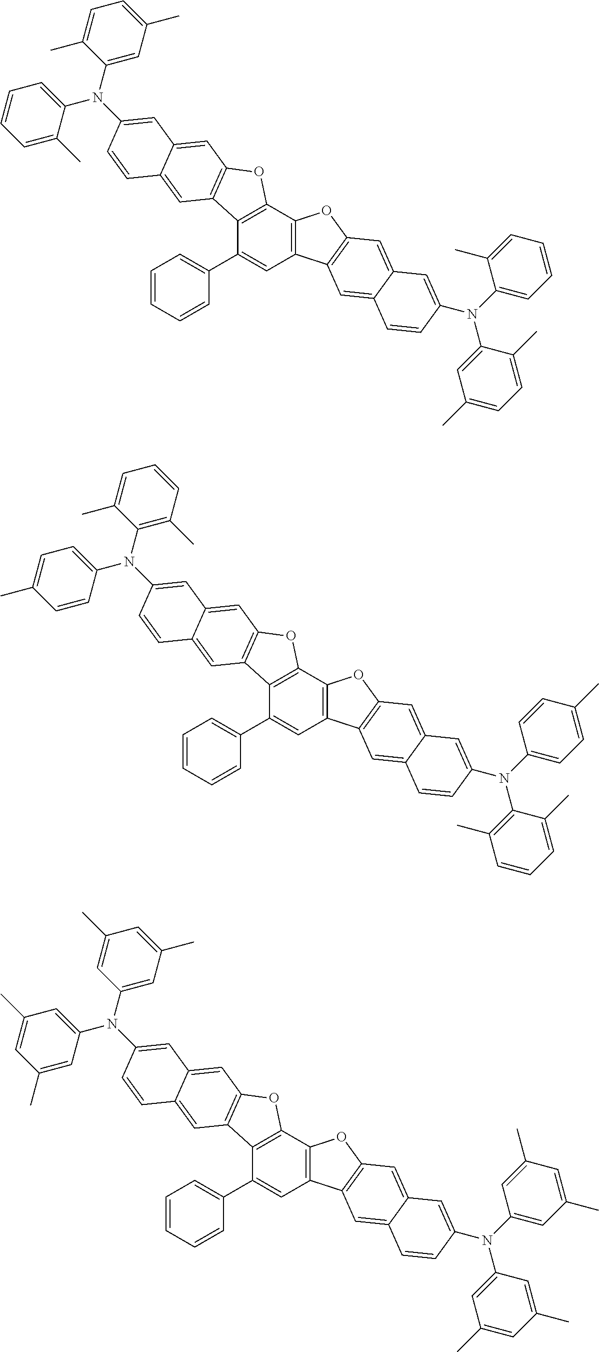

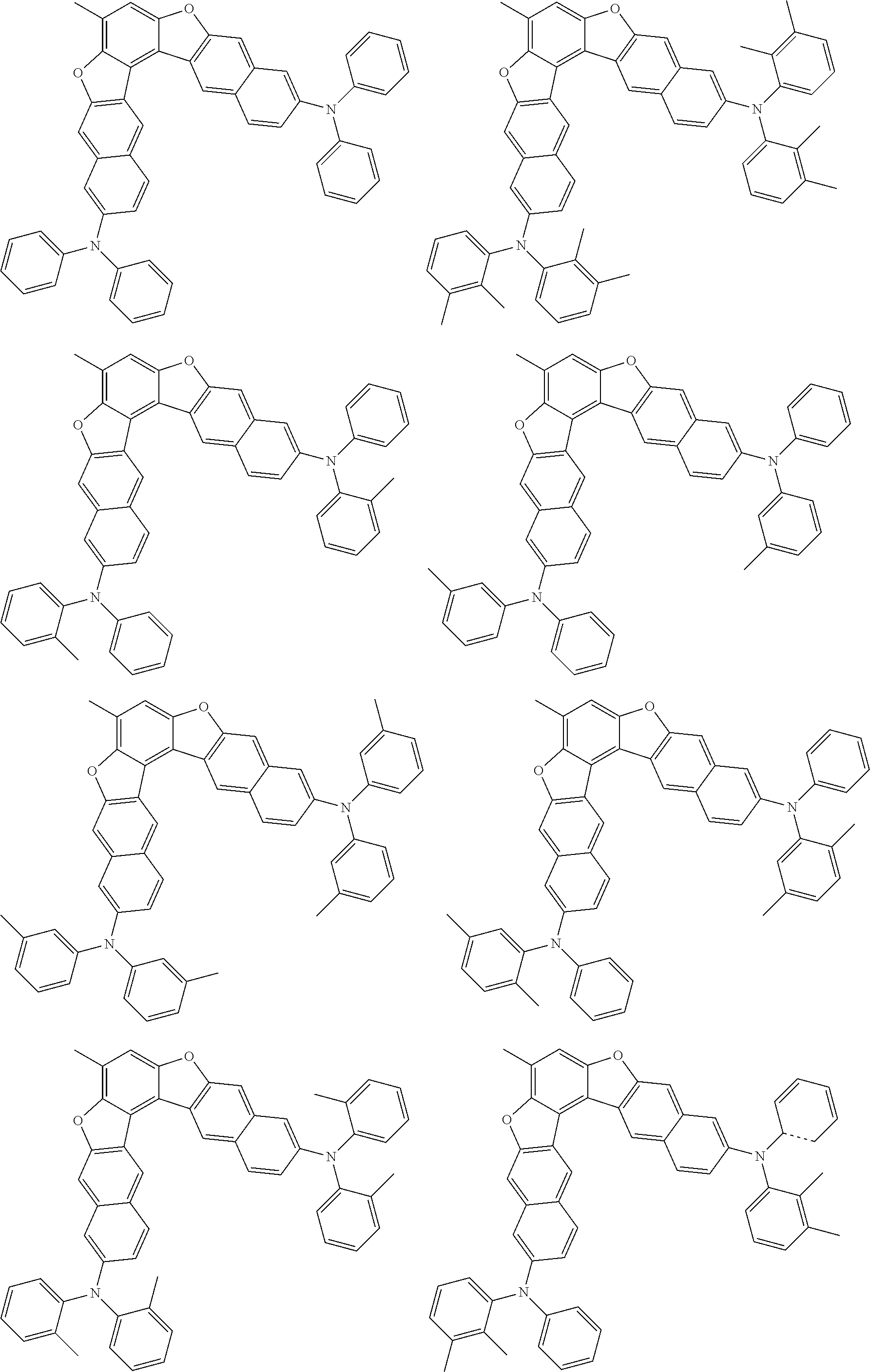

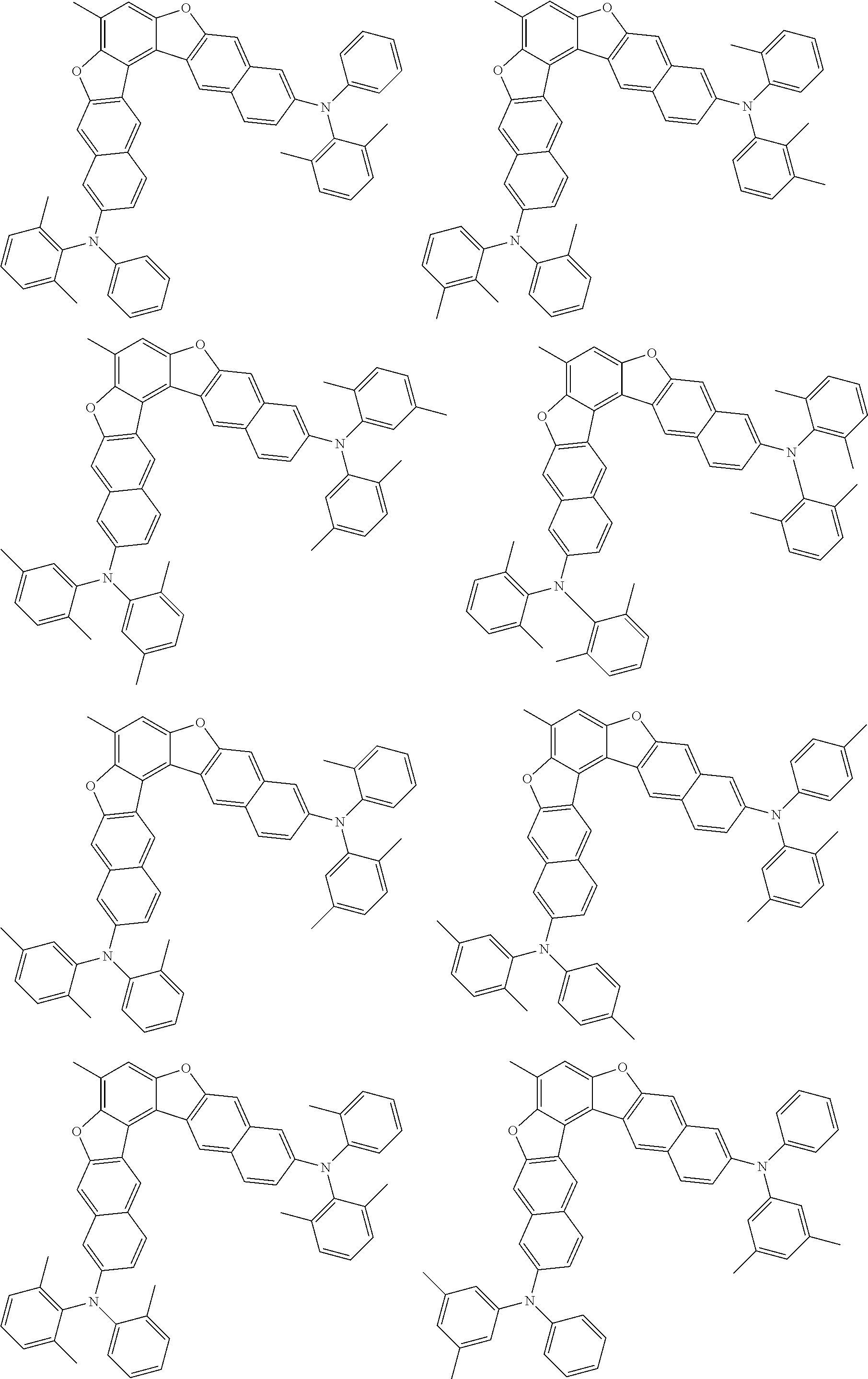

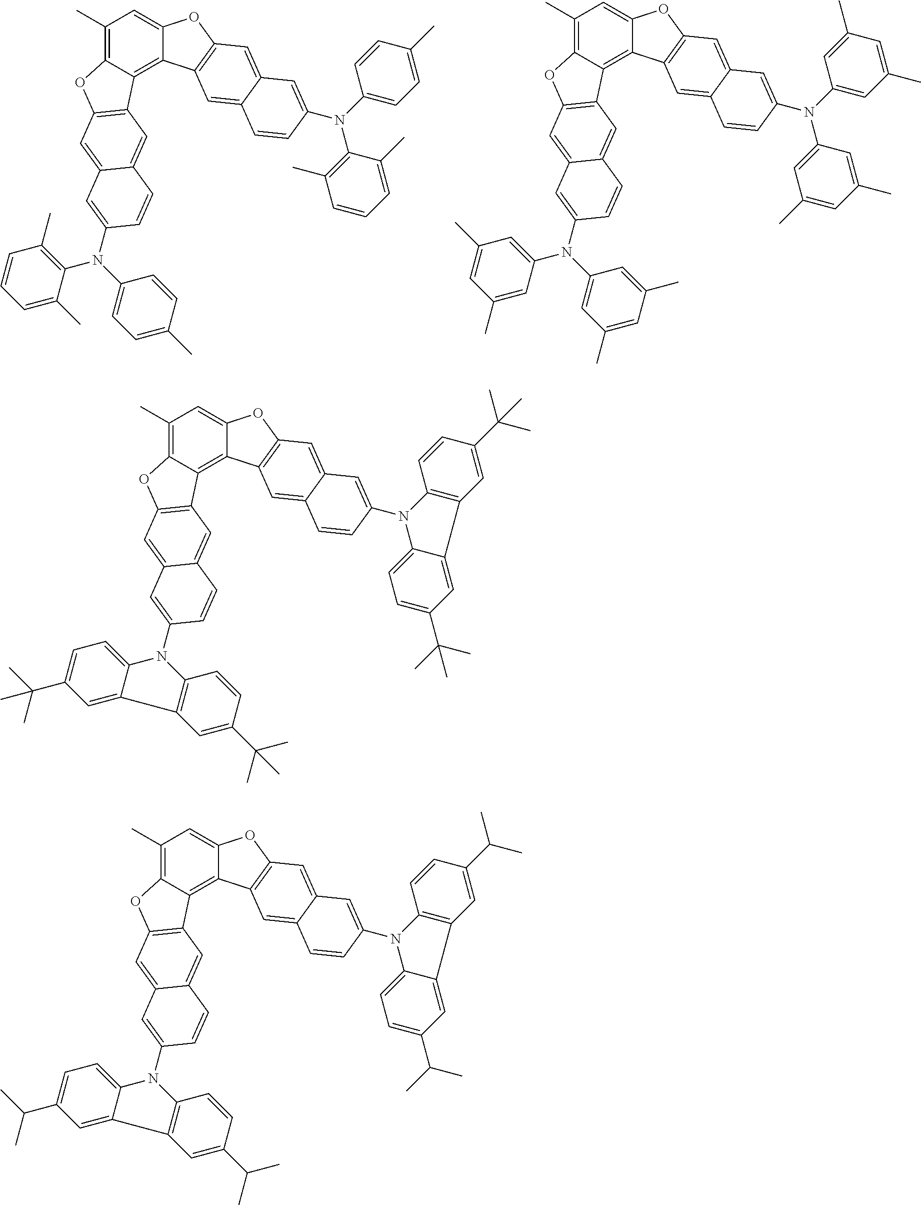

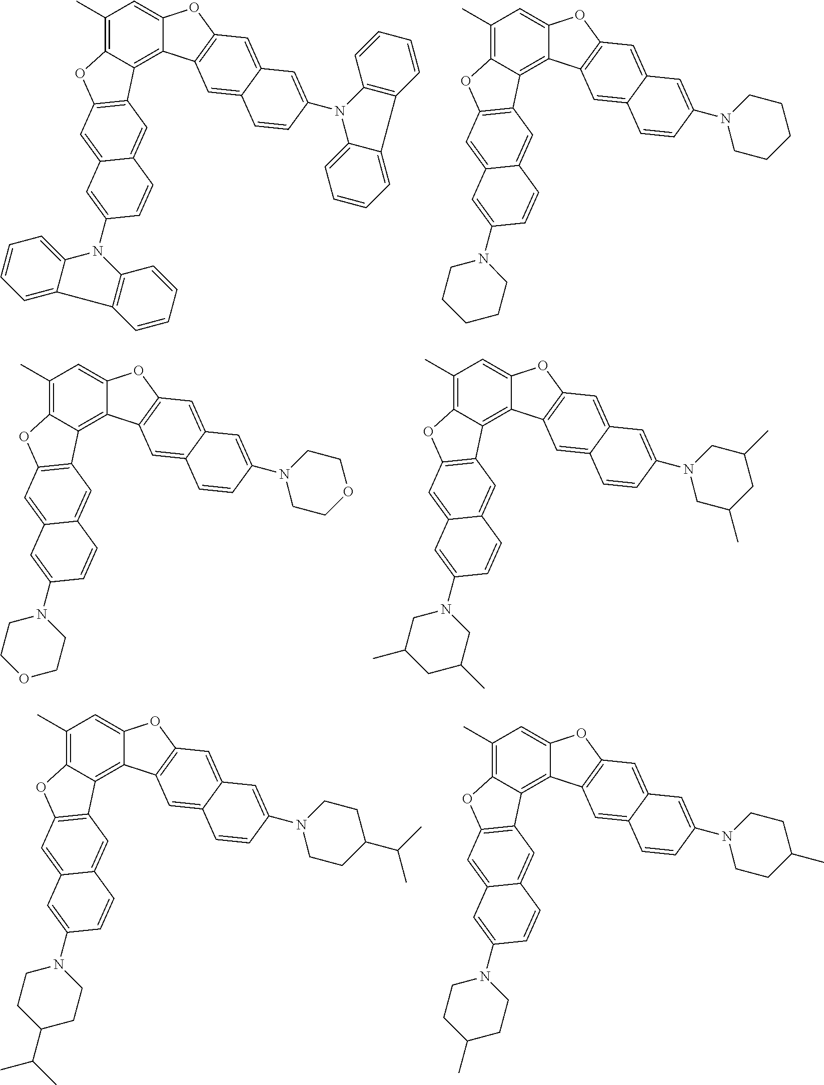

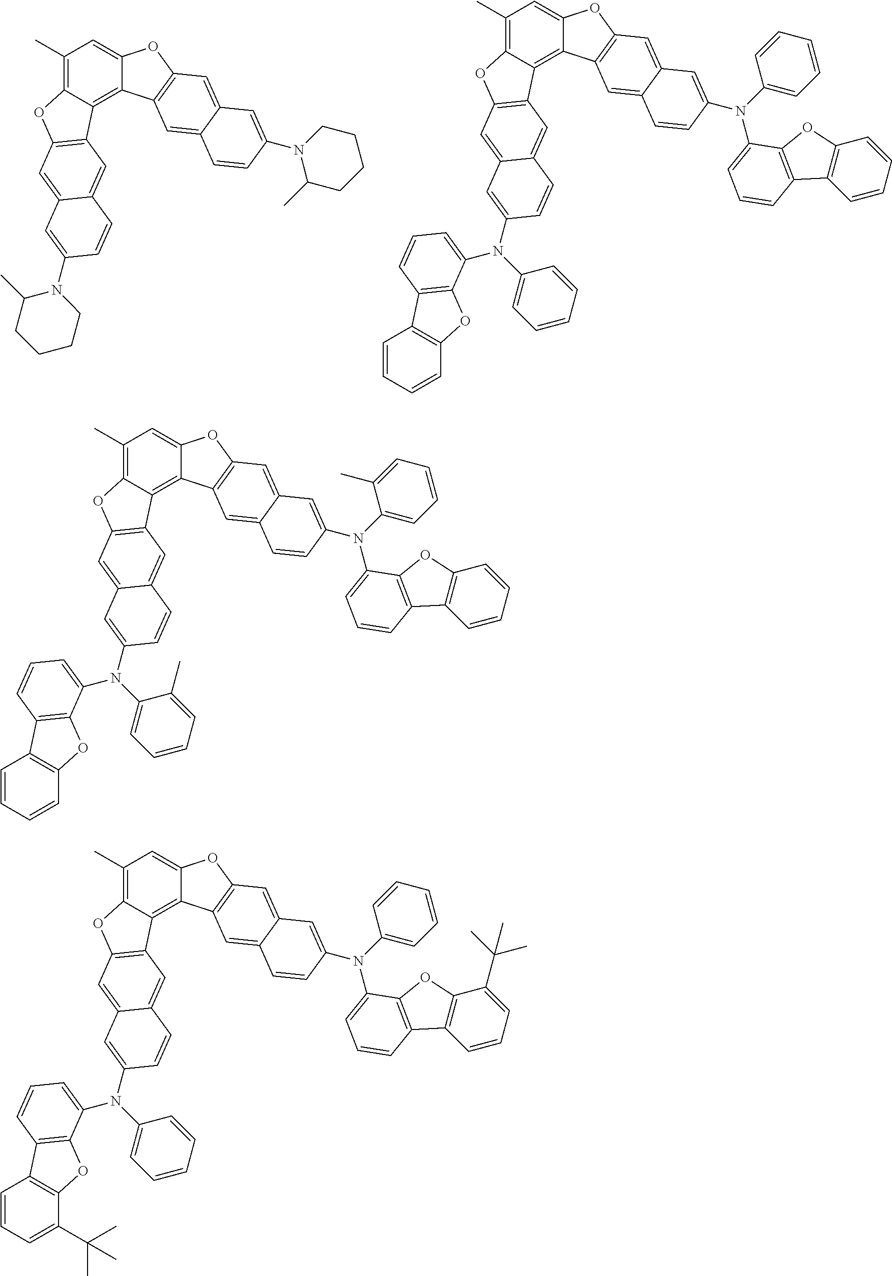 [SMILES: Cc1cc2oc3cc4cc(N5CCCCC5C)ccc4cc3c2c2c1oc1cc3cc(N4CCCCC4C)ccc3cc12 ; Cc1cc2oc3cc4cc(N(c5ccccc5)c5cccc6c5oc5ccccc56)ccc4cc3c2c2c1oc1cc3cc(N(c4ccccc4)c4cccc5c4oc4ccccc45)ccc3cc12 ; Cc1ccccc1N(c1ccc2cc3c(cc2c1)oc1cc(C)c2oc4cc5cc(N(c6ccccc6C)c6cccc7c6oc6ccccc67)ccc5cc4c2c13)c1cccc2c1oc1ccccc12 ; Cc1cc2oc3cc4cc(N(c5ccccc5)c5cccc6c5oc5c(C(C)(C)C)cccc56)ccc4cc3c2c2c1oc1cc3cc(N(c4ccccc4)c4cccc5c4oc4c(C(C)(C)C)cccc45)ccc3cc12]

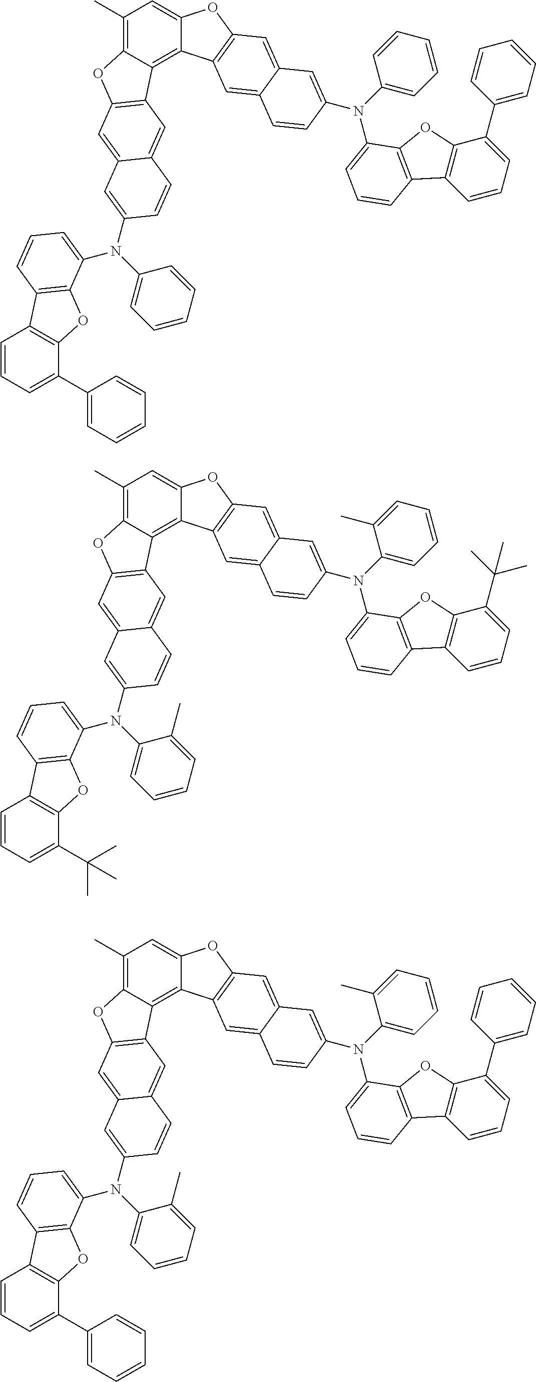

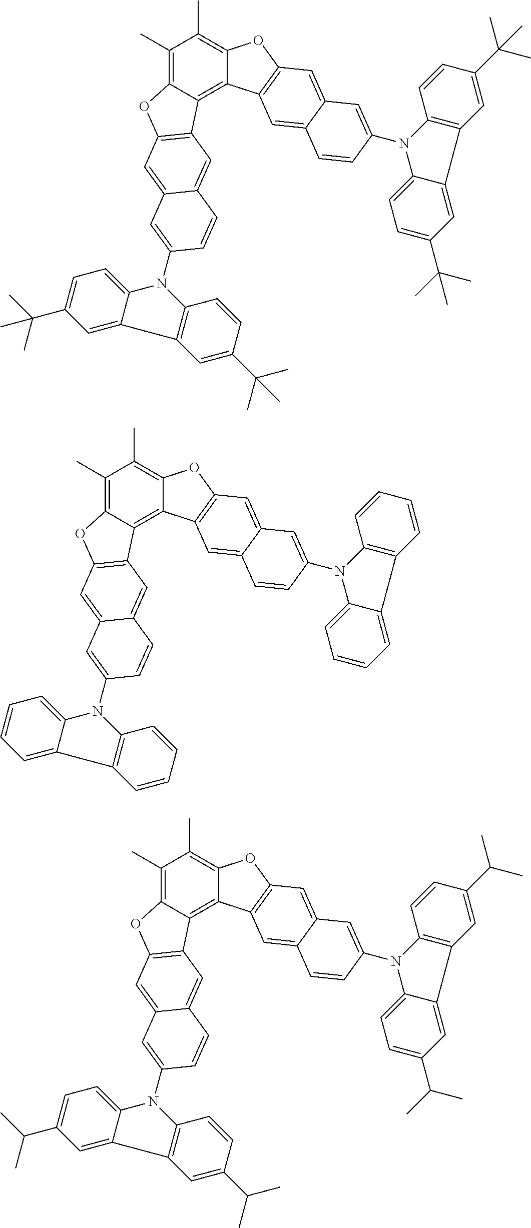

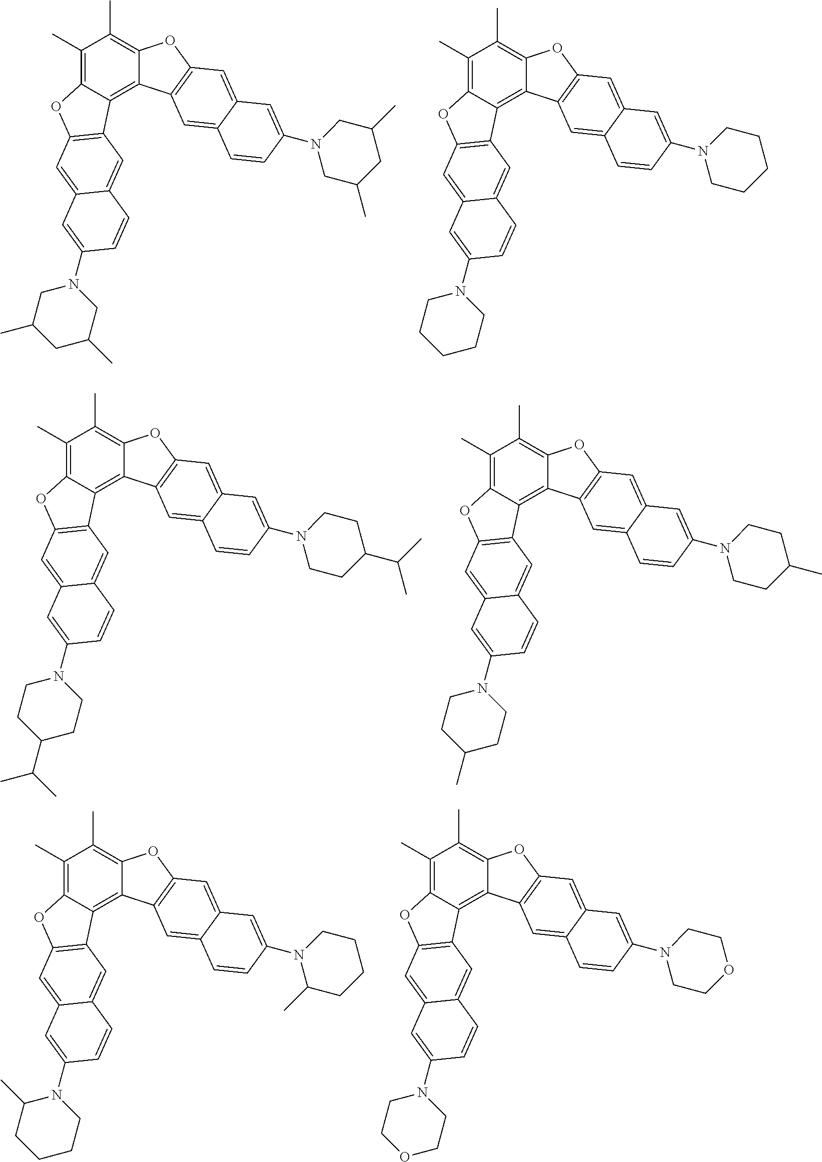

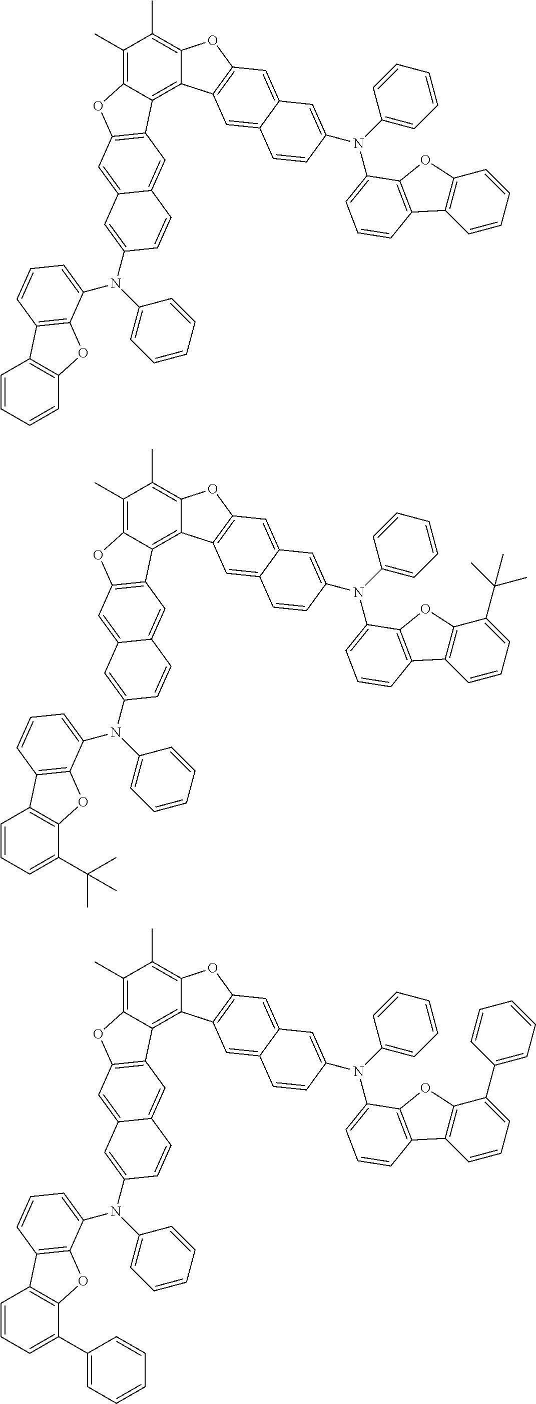 [SMILES: Cc1c(C)c2oc3cc4cc(N(c5ccccc5)c5cccc6c5oc5ccccc56)ccc4cc3c2c2c1oc1cc3cc(N(c4ccccc4)c4cccc5c4oc4ccccc45)ccc3cc12 ; Cc1c(C)c2oc3cc4cc(N(c5ccccc5)c5cccc6c5oc5c(C(C)(C)C)cccc56)ccc4cc3c2c2c1oc1cc3cc(N(c4ccccc4)c4cccc5c4oc4c(C(C)(C)C)cccc45)ccc3cc12 ; Cc1c(C)c2oc3cc4cc(N(c5ccccc5)c5cccc6c5oc5c(-c7ccccc7)cccc56)ccc4cc3c2c2c1oc1cc3cc(N(c4ccccc4)c4cccc5c4oc4c(-c6ccccc6)cccc45)ccc3cc12]

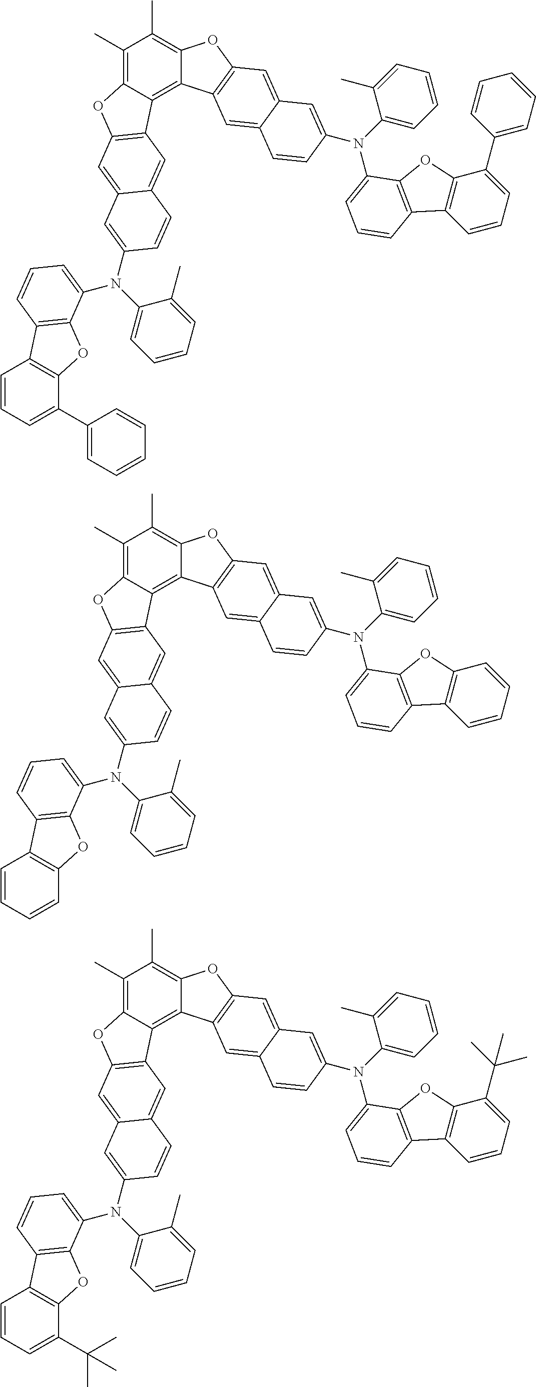

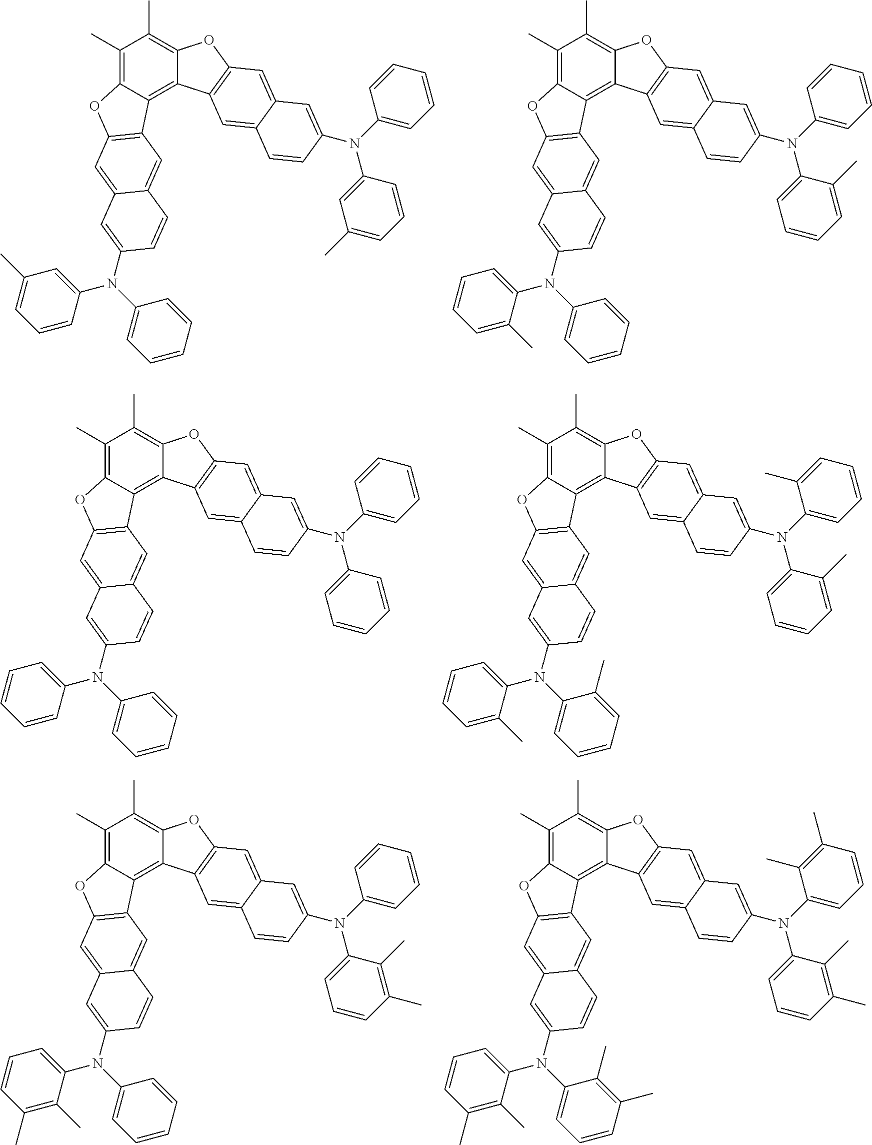

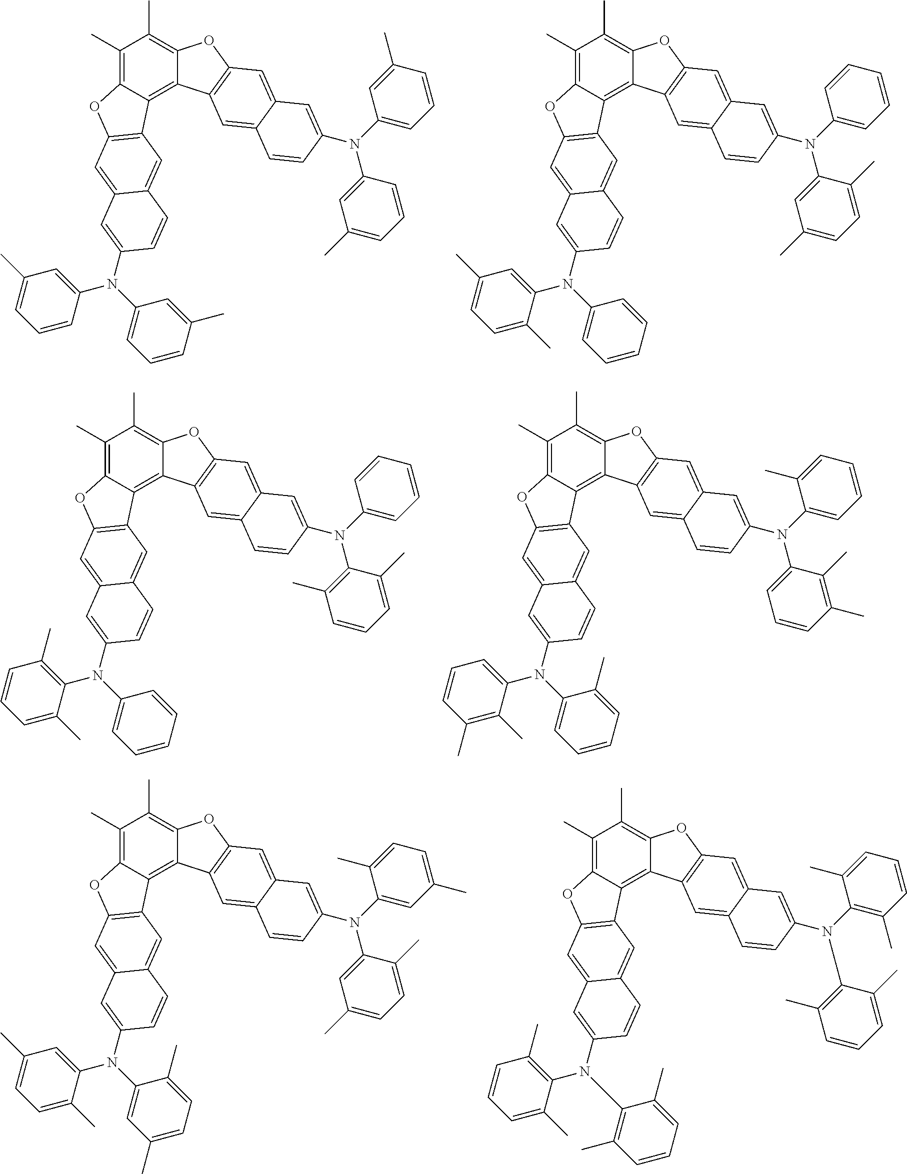

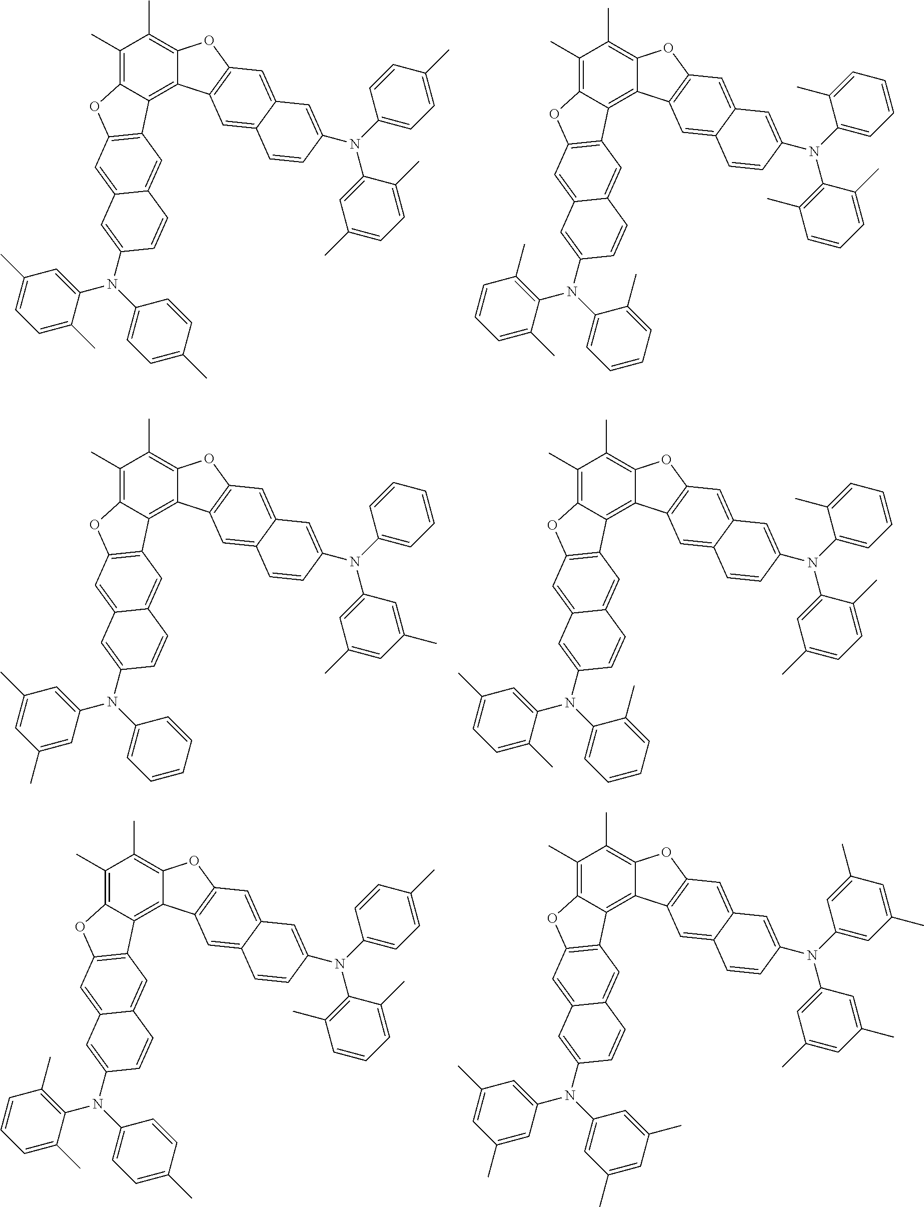

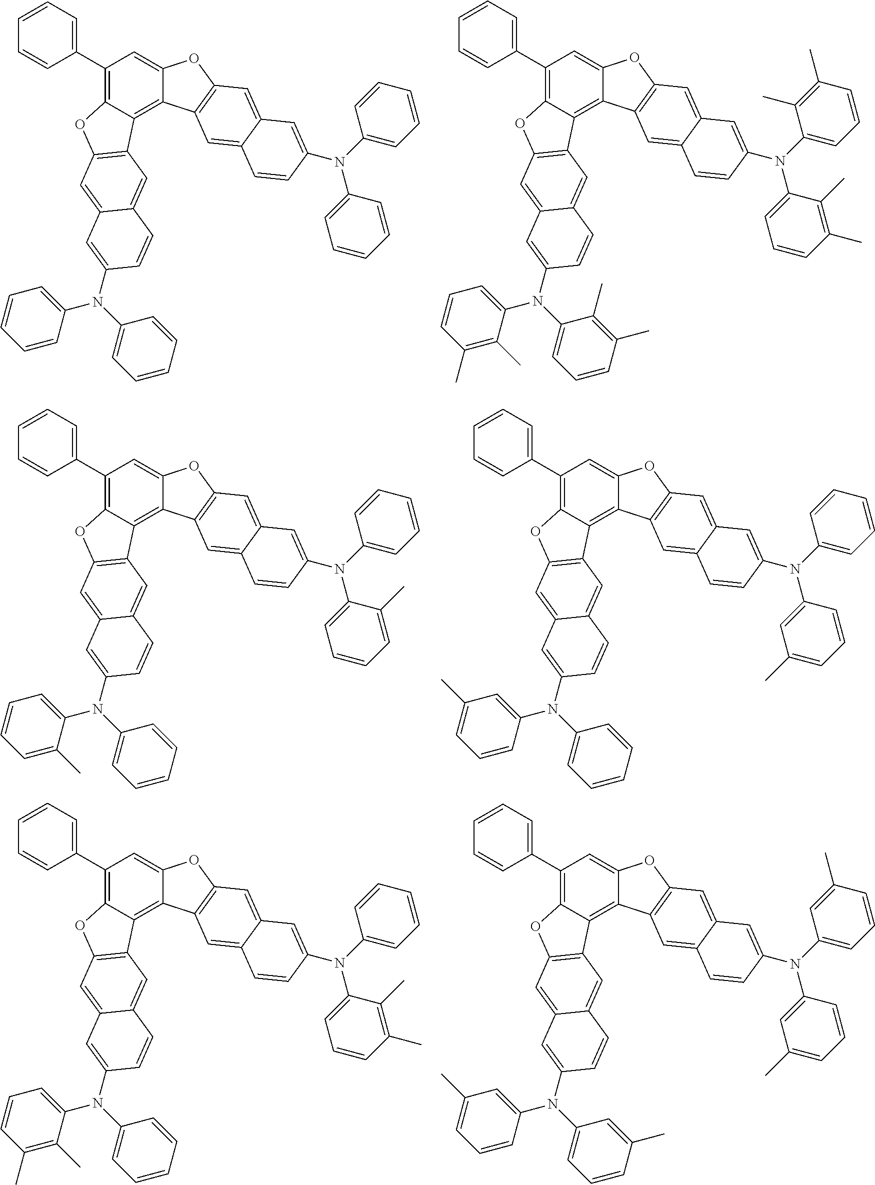

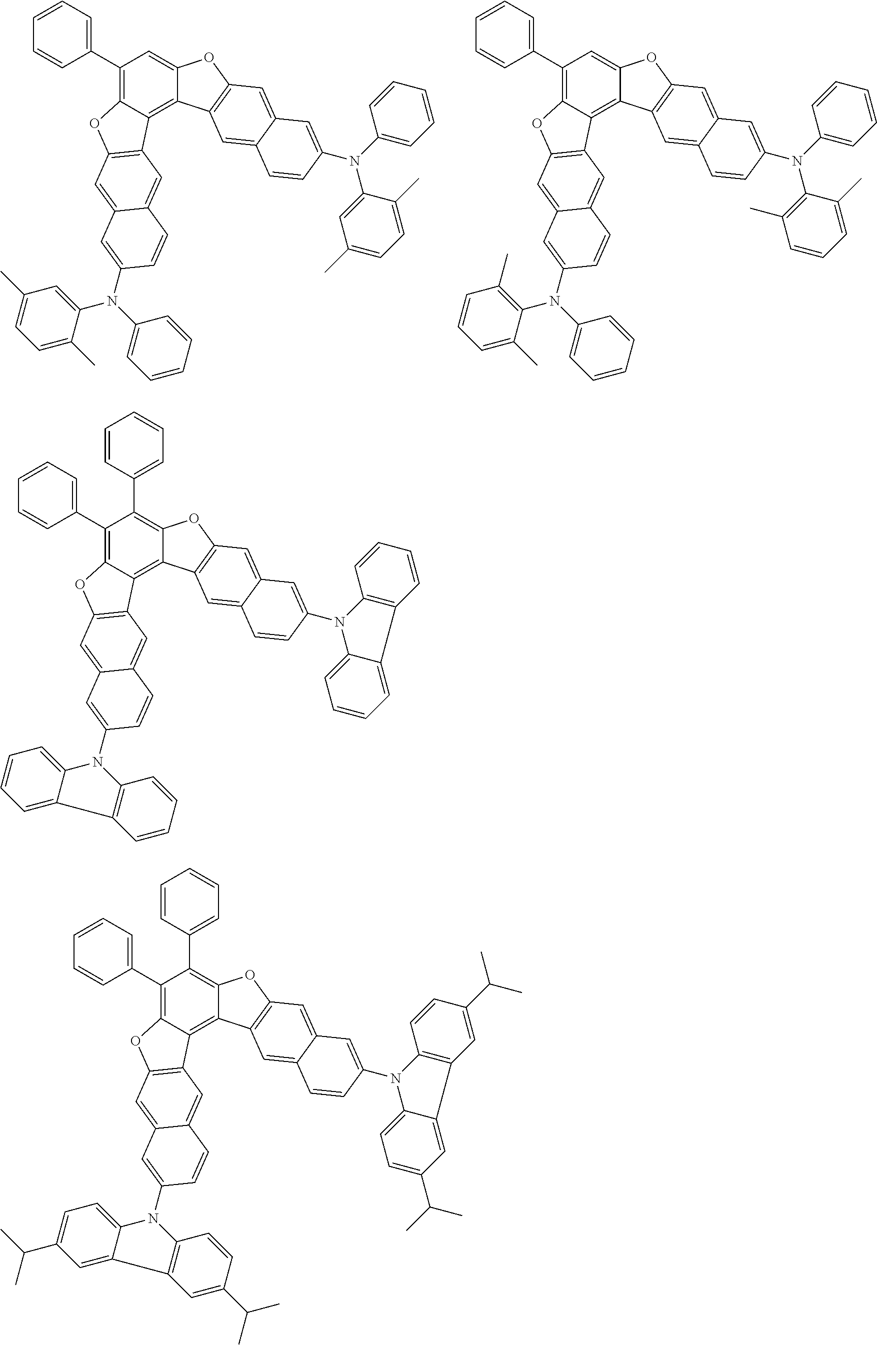

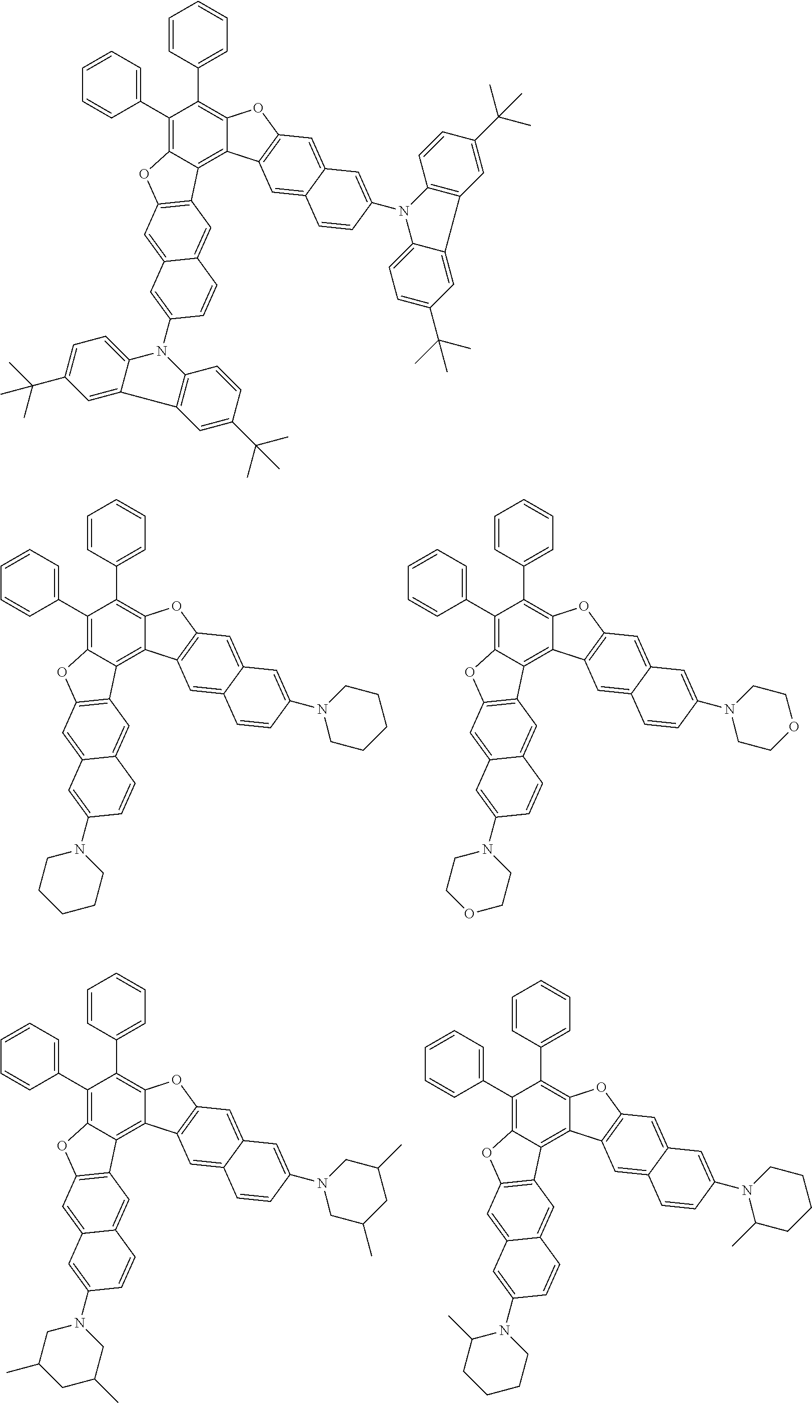

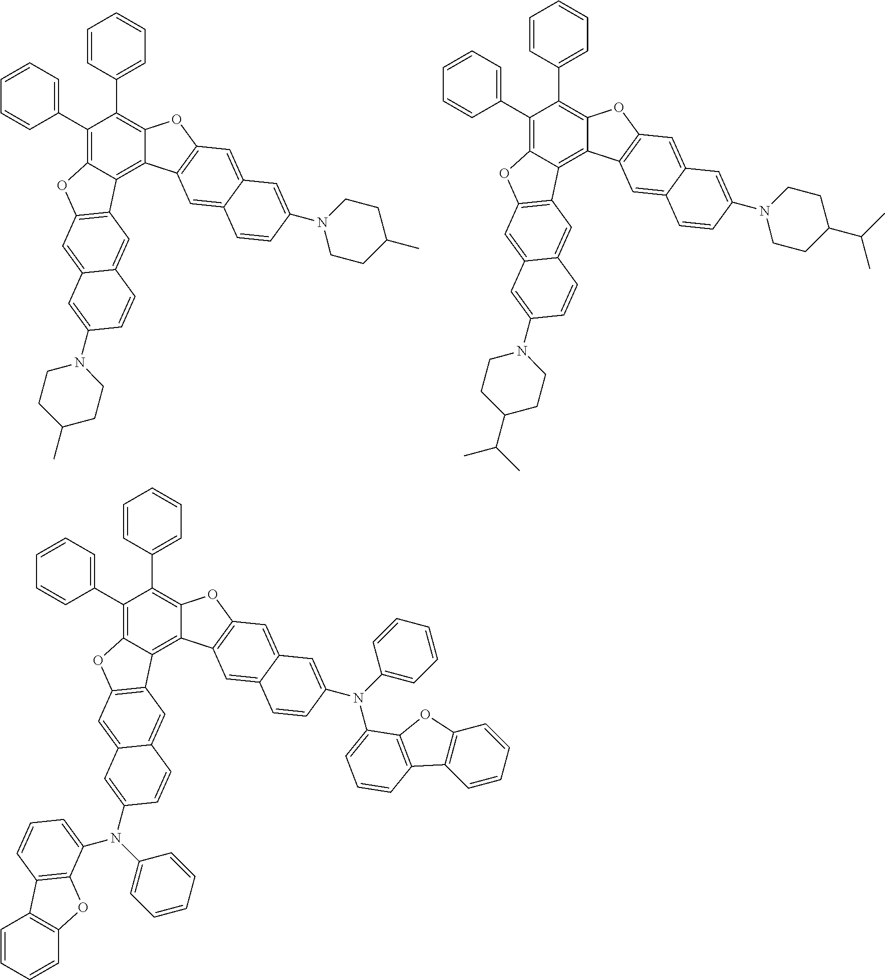 [SMILES: CC1CCN(c2ccc3cc4c(cc3c2)oc2c(-c3ccccc3)c(-c3ccccc3)c3oc5cc6cc(N7CCC(C)CC7)ccc6cc5c3c24)CC1 ; CC(C)C1CCN(c2ccc3cc4c(cc3c2)oc2c(-c3ccccc3)c(-c3ccccc3)c3oc5cc6cc(N7CCC(C(C)C)CC7)ccc6cc5c3c24)CC1 ; c1ccc(-c2c(-c3ccccc3)c3oc4cc5cc(N(c6ccccc6)c6cccc7c6oc6ccccc67)ccc5cc4c3c3c2oc2cc4cc(N(c5ccccc5)c5cccc6c5oc5ccccc56)ccc4cc23)cc1]

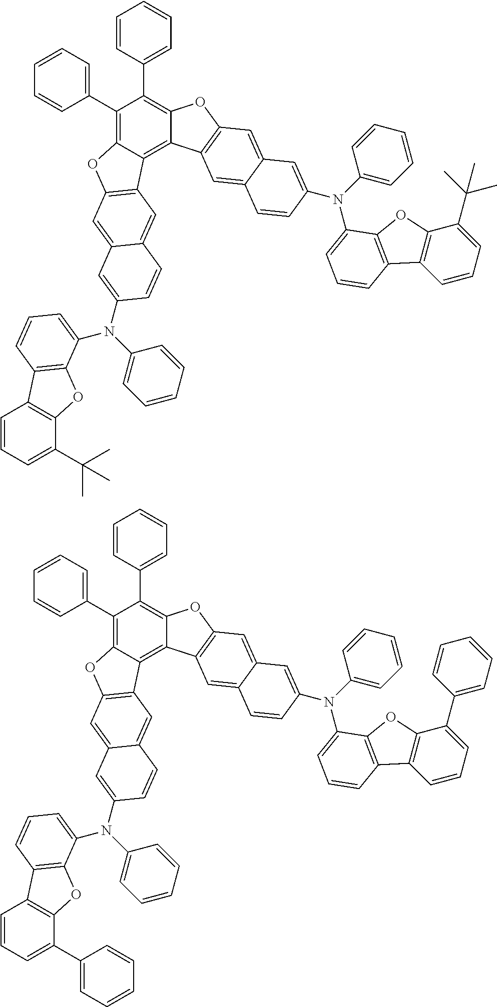

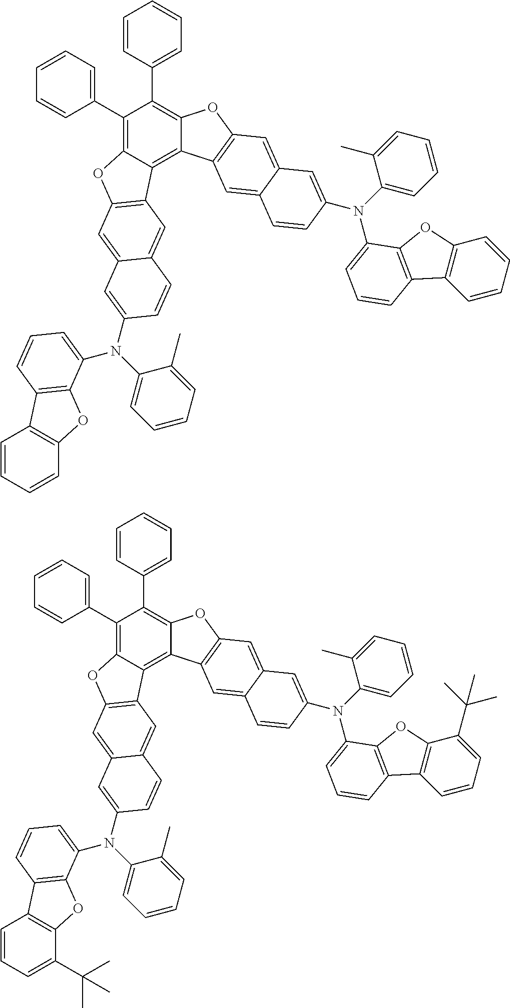 [SMILES: Cc1ccccc1N(c1ccc2cc3c(cc2c1)oc1c(-c2ccccc2)c(-c2ccccc2)c2oc4cc5cc(N(c6ccccc6C)c6cccc7c6oc6ccccc67)ccc5cc4c2c13)c1cccc2c1oc1ccccc12 ; Cc1ccccc1N(c1ccc2cc3c(cc2c1)oc1c(-c2ccccc2)c(-c2ccccc2)c2oc4cc5cc(N(c6ccccc6C)c6cccc7c6oc6c(C(C)(C)C)cccc67)ccc5cc4c2c13)c1cccc2c1oc1c(C(C)(C)C)cccc12]

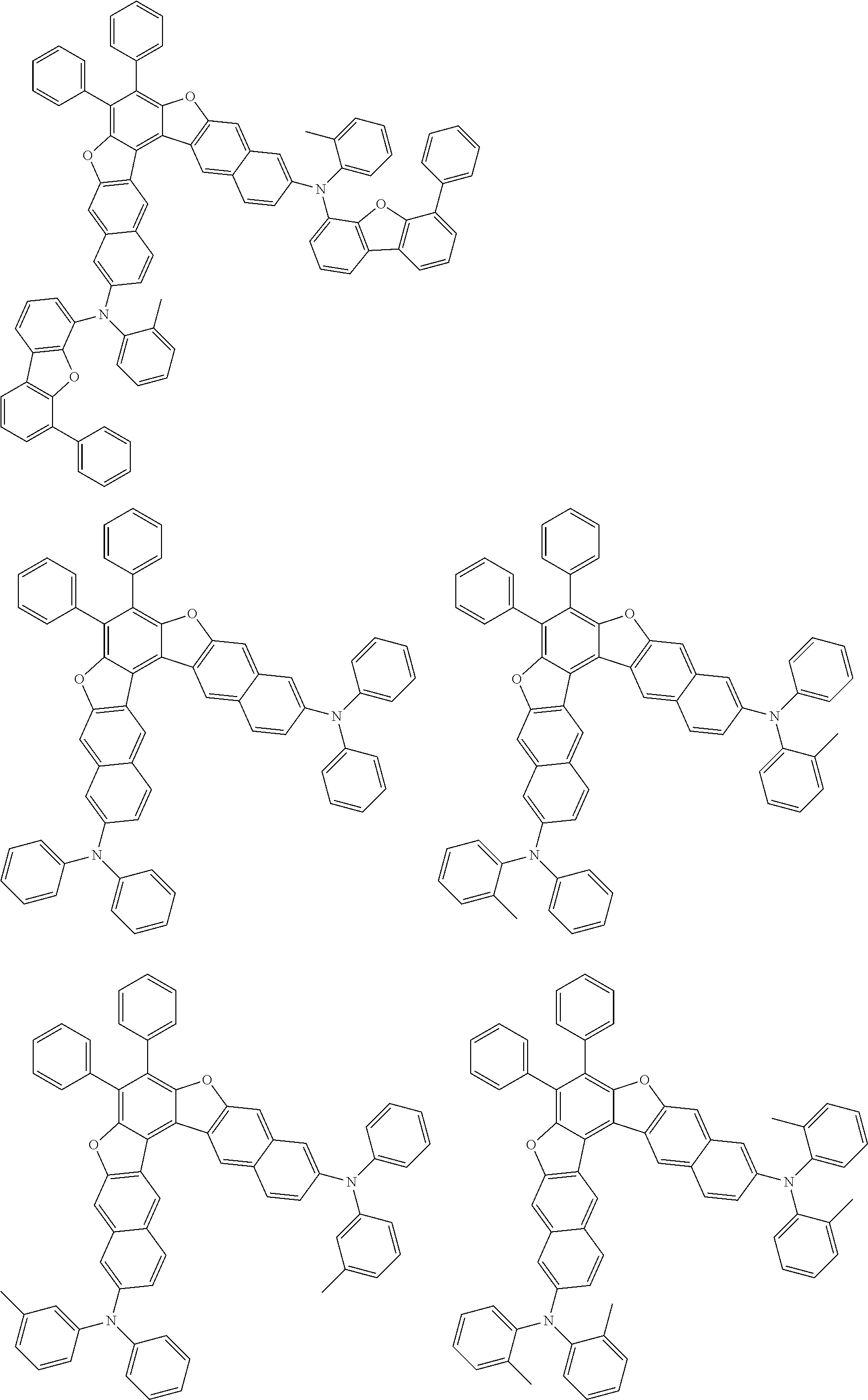

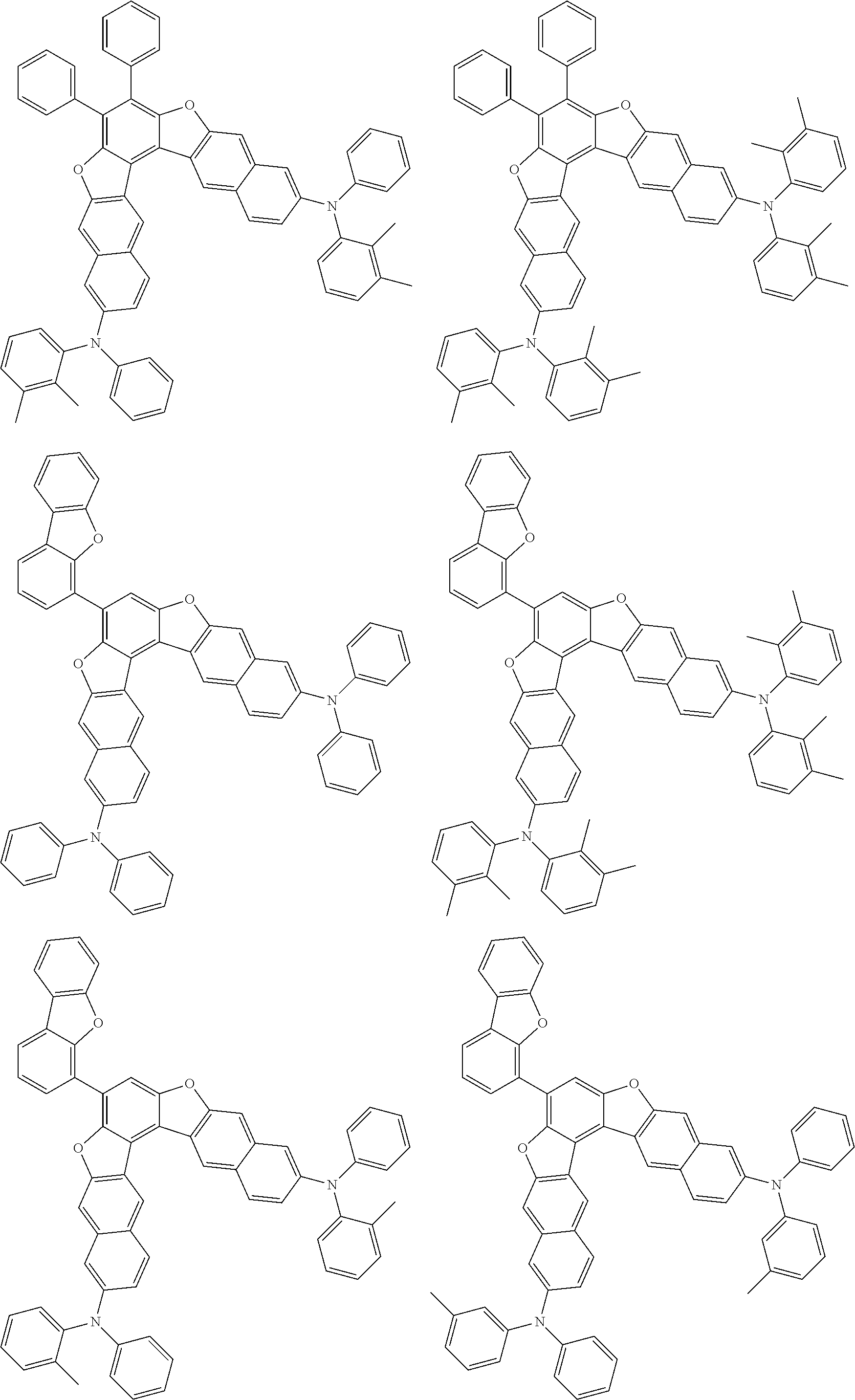

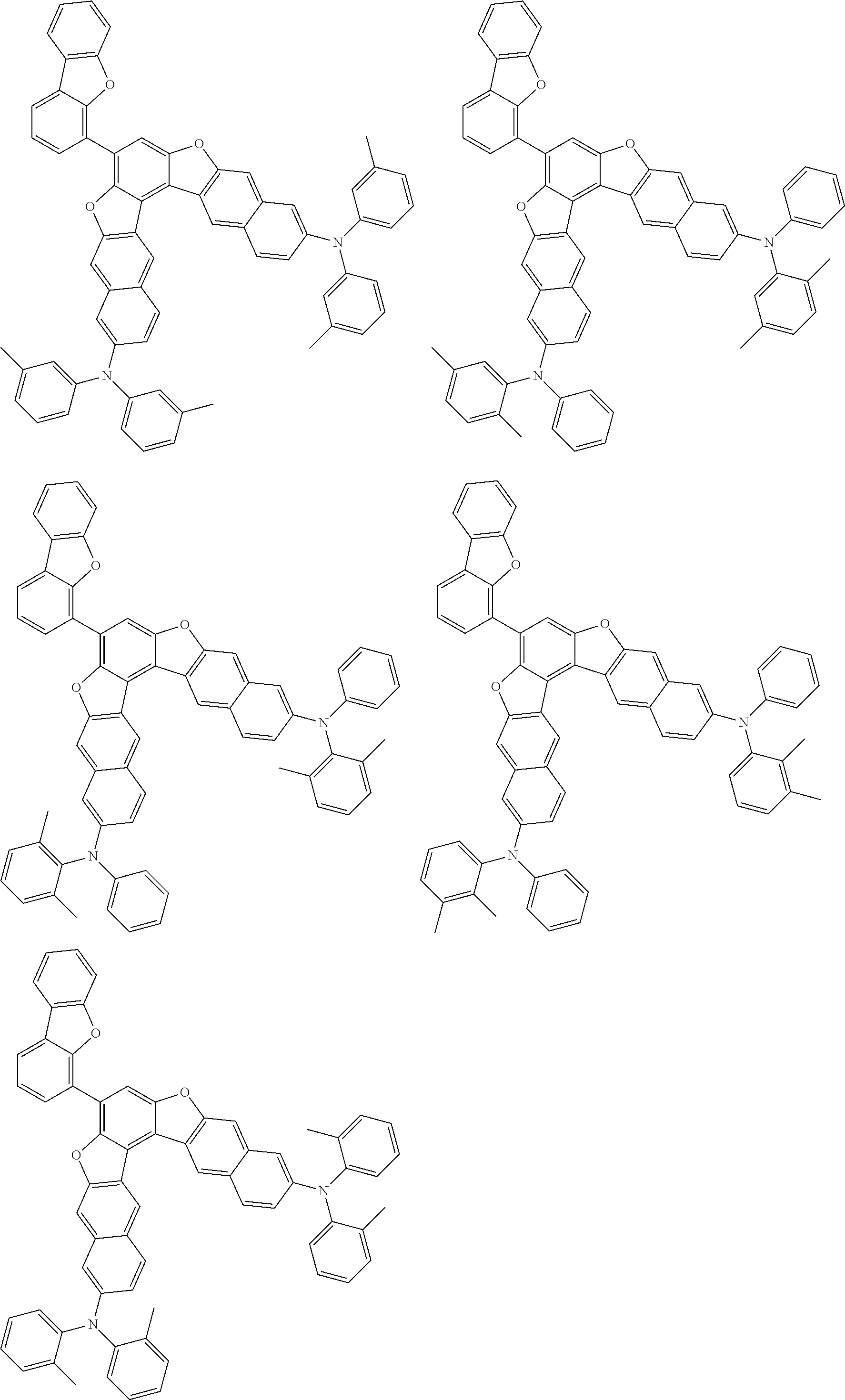

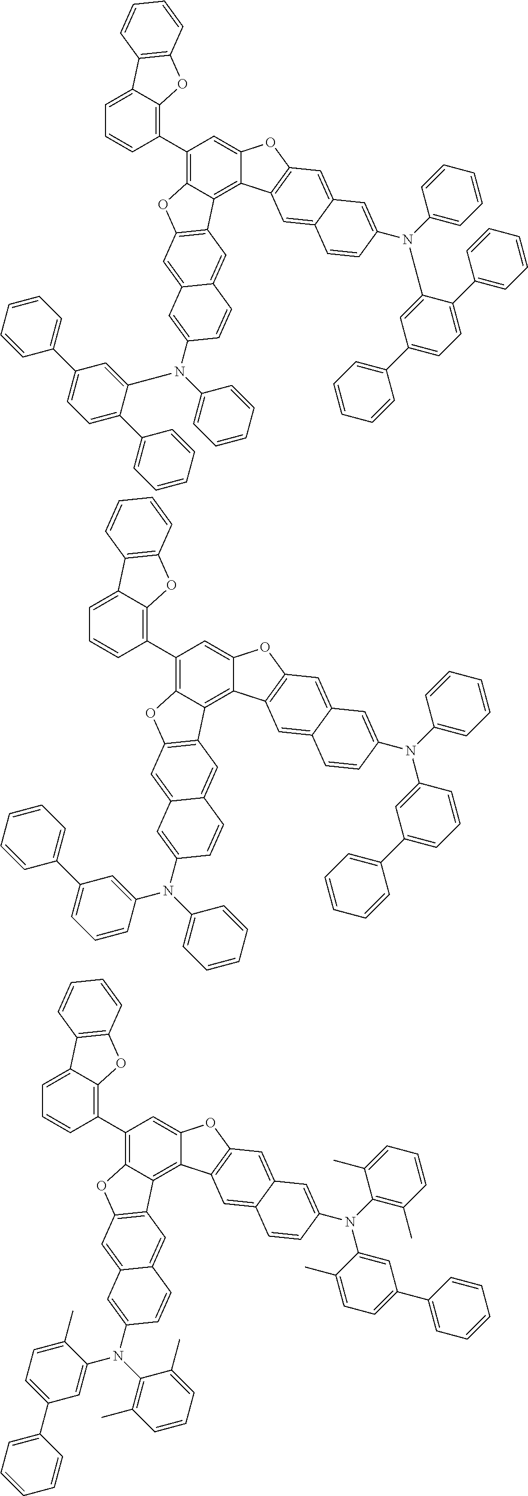

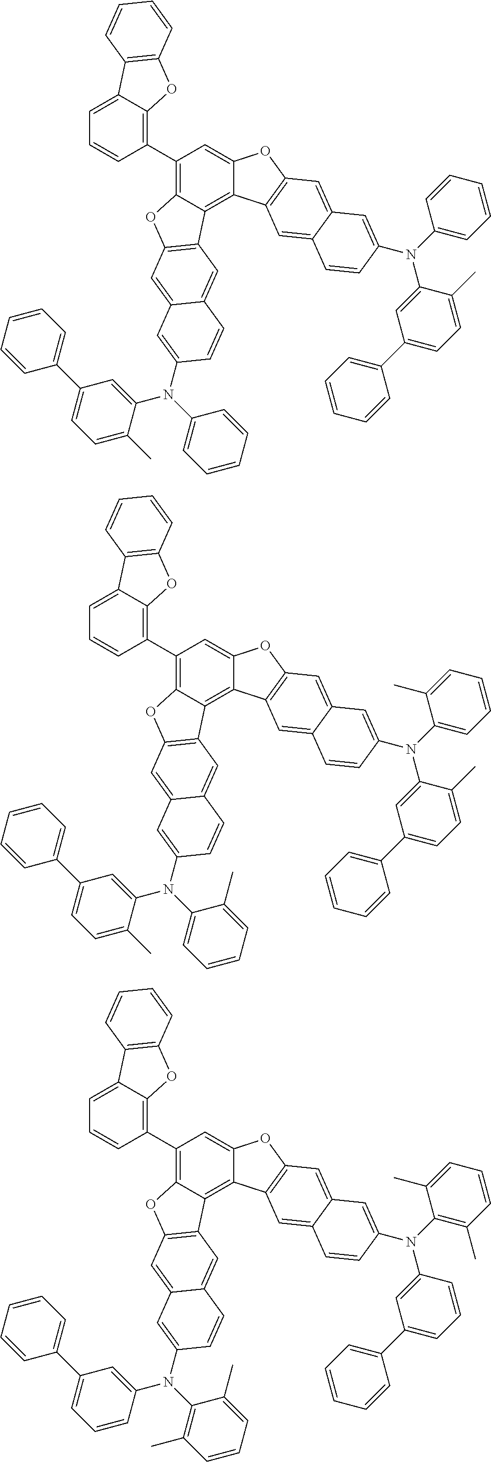

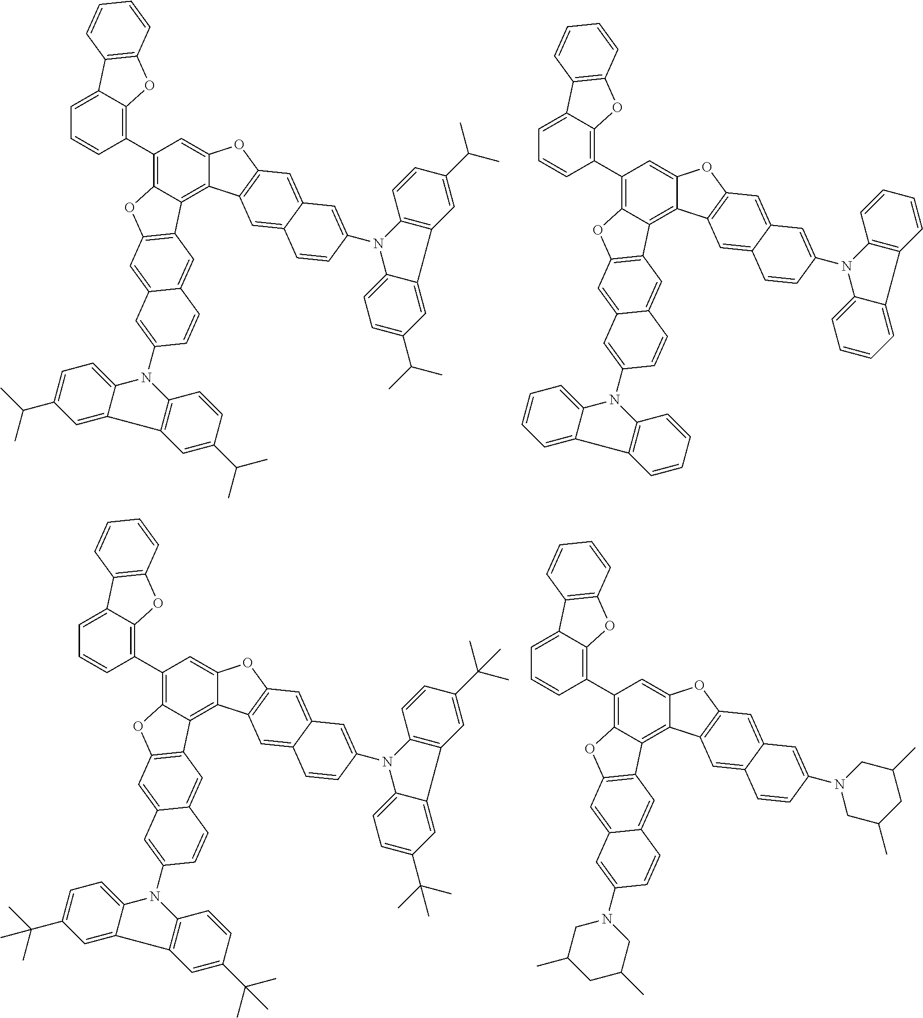

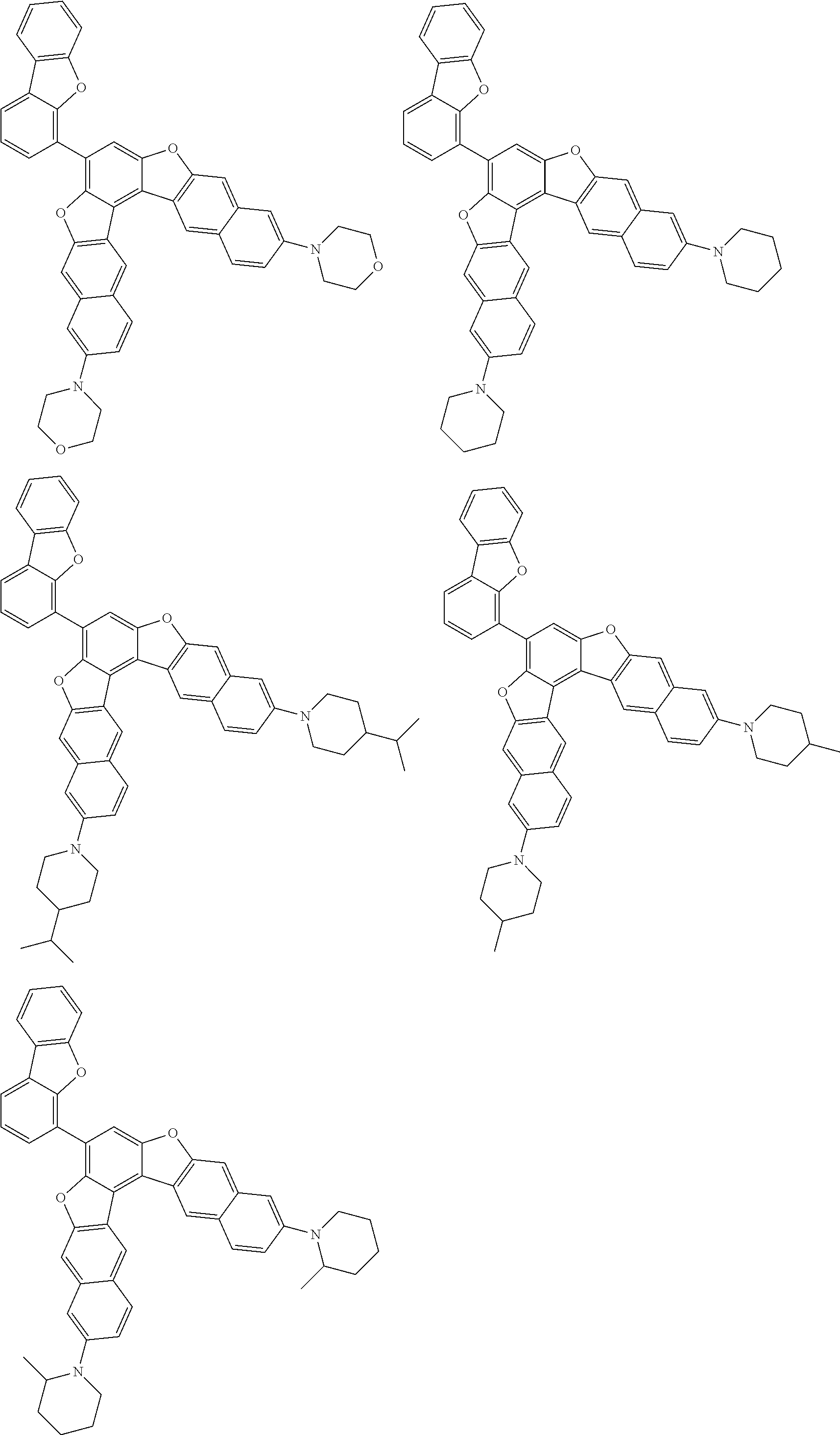

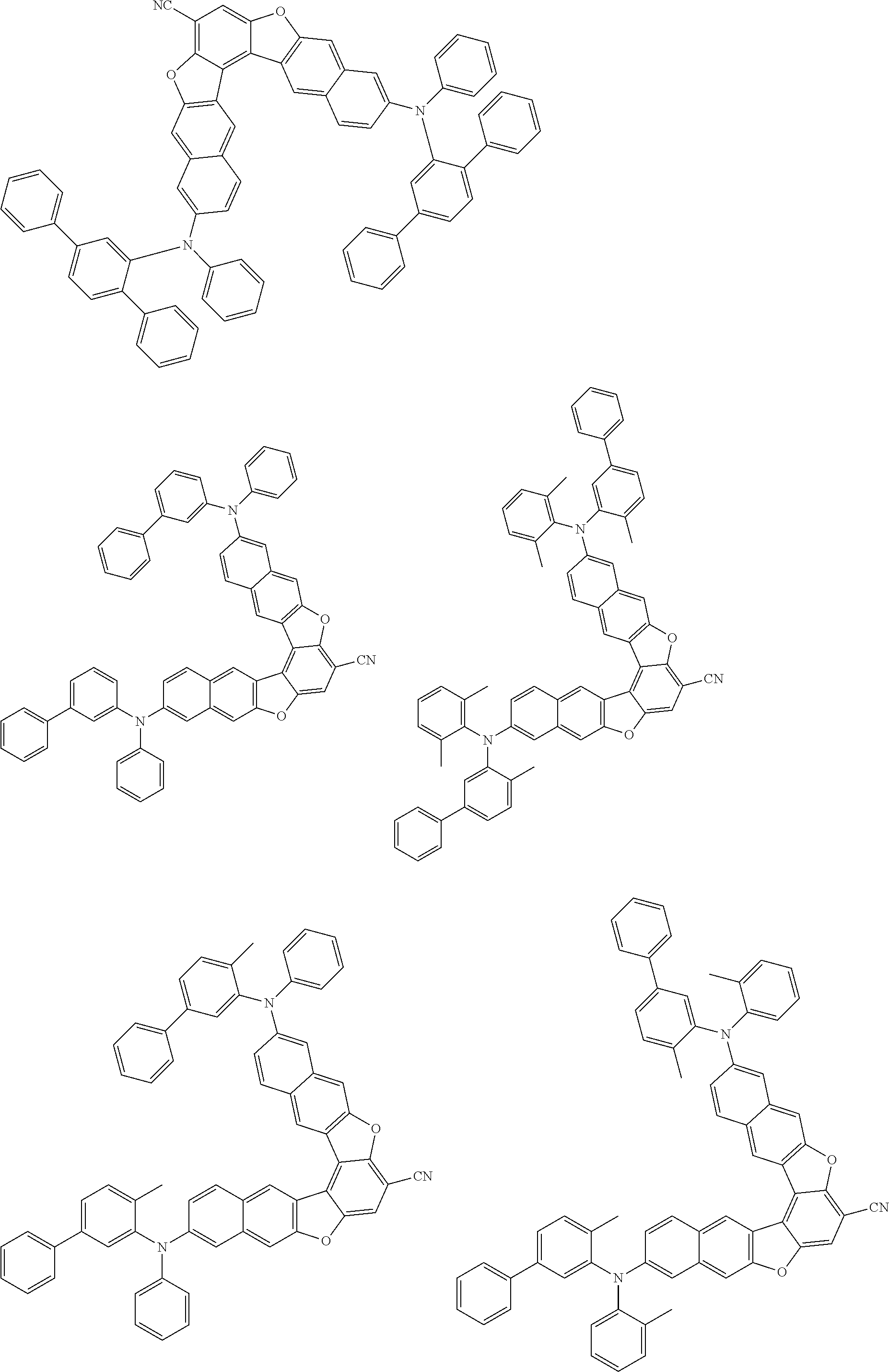

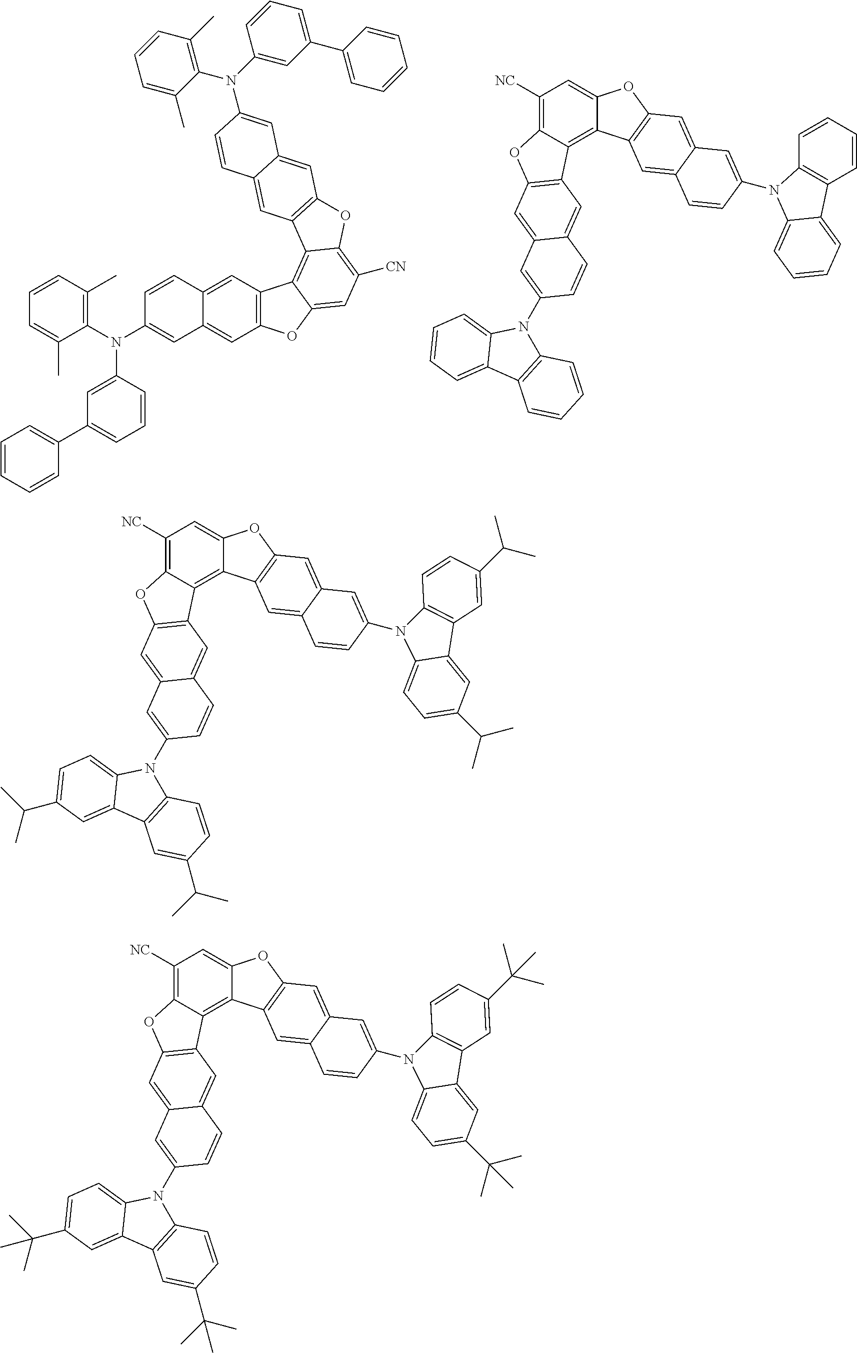

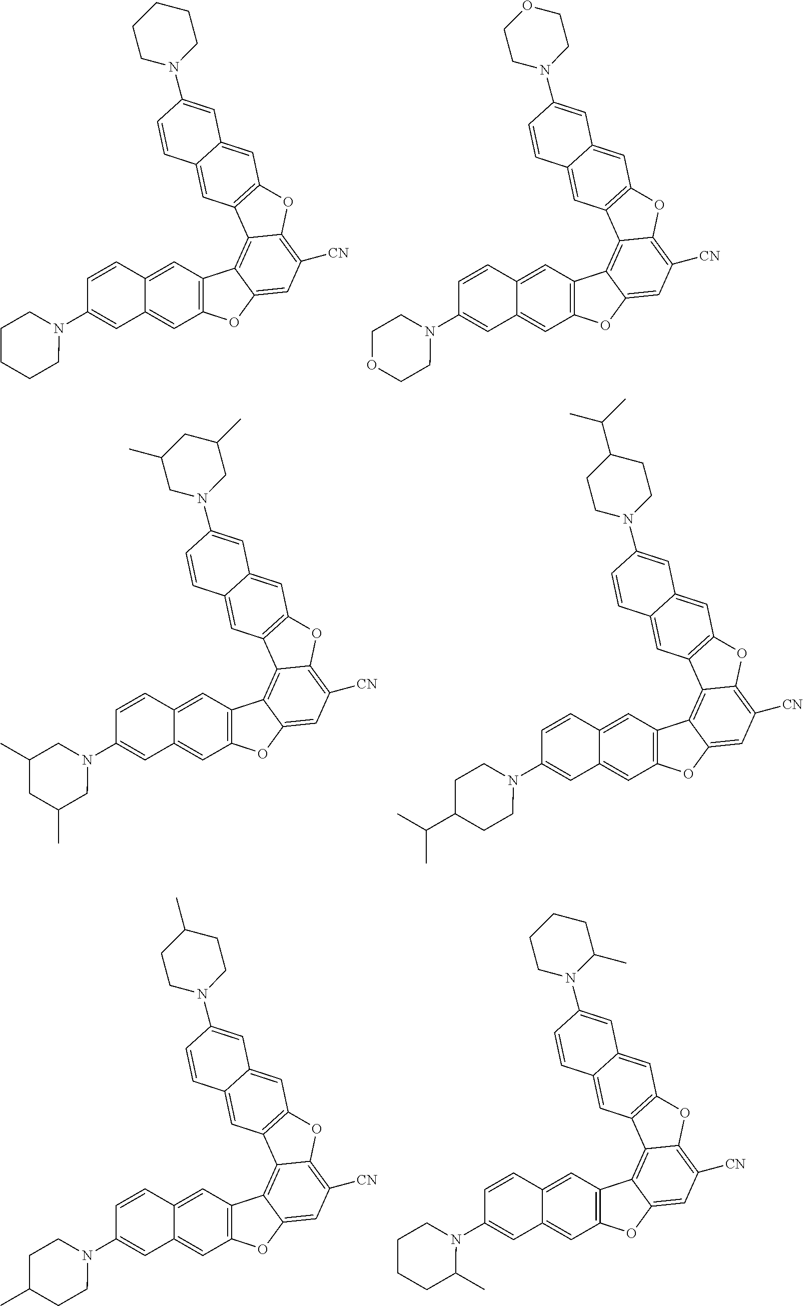 [SMILES: N#Cc1cc2oc3cc4cc(N5CCCCC5)ccc4cc3c2c2c1oc1cc3cc(N4CCCCC4)ccc3cc12 ; N#Cc1cc2oc3cc4cc(N5CCOCC5)ccc4cc3c2c2c1oc1cc3cc(N4CCOCC4)ccc3cc12 ; CC1CC(C)CN(c2ccc3cc4c(cc3c2)oc2cc(C#N)c3oc5cc6cc(N7CC(C)CC(C)C7)ccc6cc5c3c24)C1 ; CC(C)C1CCN(c2ccc3cc4c(cc3c2)oc2cc(C#N)c3oc5cc6cc(N7CCC(C(C)C)CC7)ccc6cc5c3c24)CC1 ; CC1CCN(c2ccc3cc4c(cc3c2)oc2cc(C#N)c3oc5cc6cc(N7CCC(C)CC7)ccc6cc5c3c24)CC1 ; CC1CCCCN1c1ccc2cc3c(cc2c1)oc1cc(C#N)c2oc4cc5cc(N6CCCCC6C)ccc5cc4c2c13]

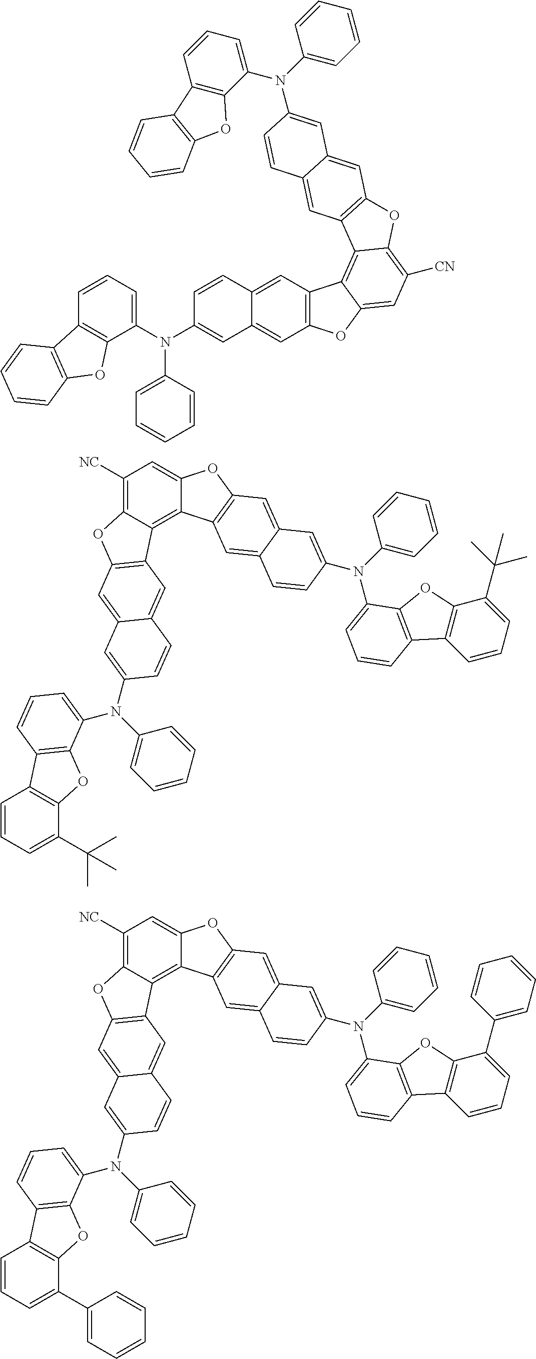

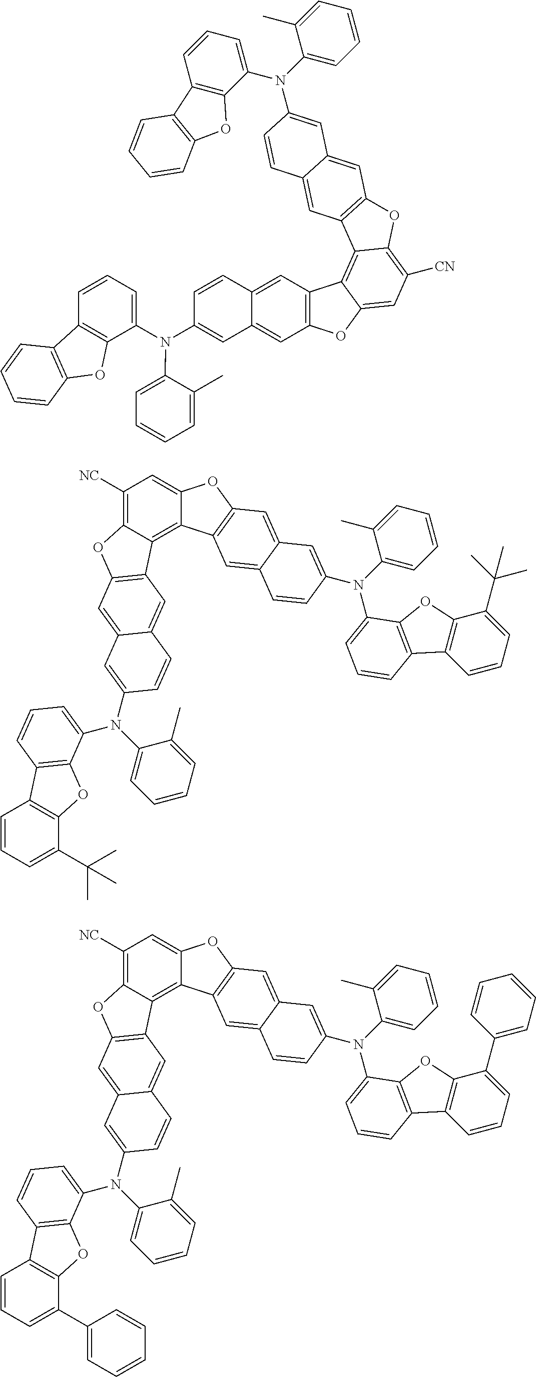

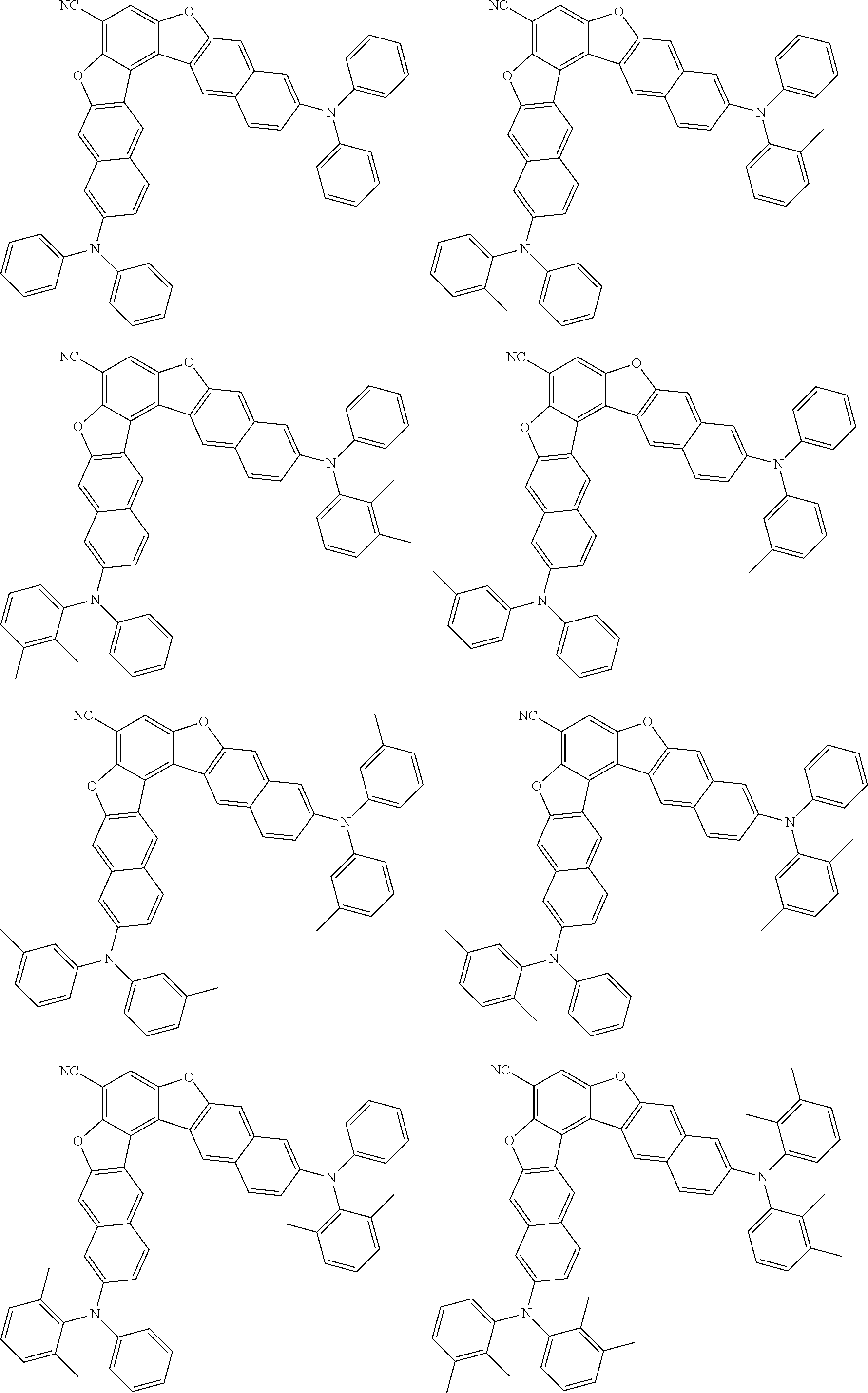

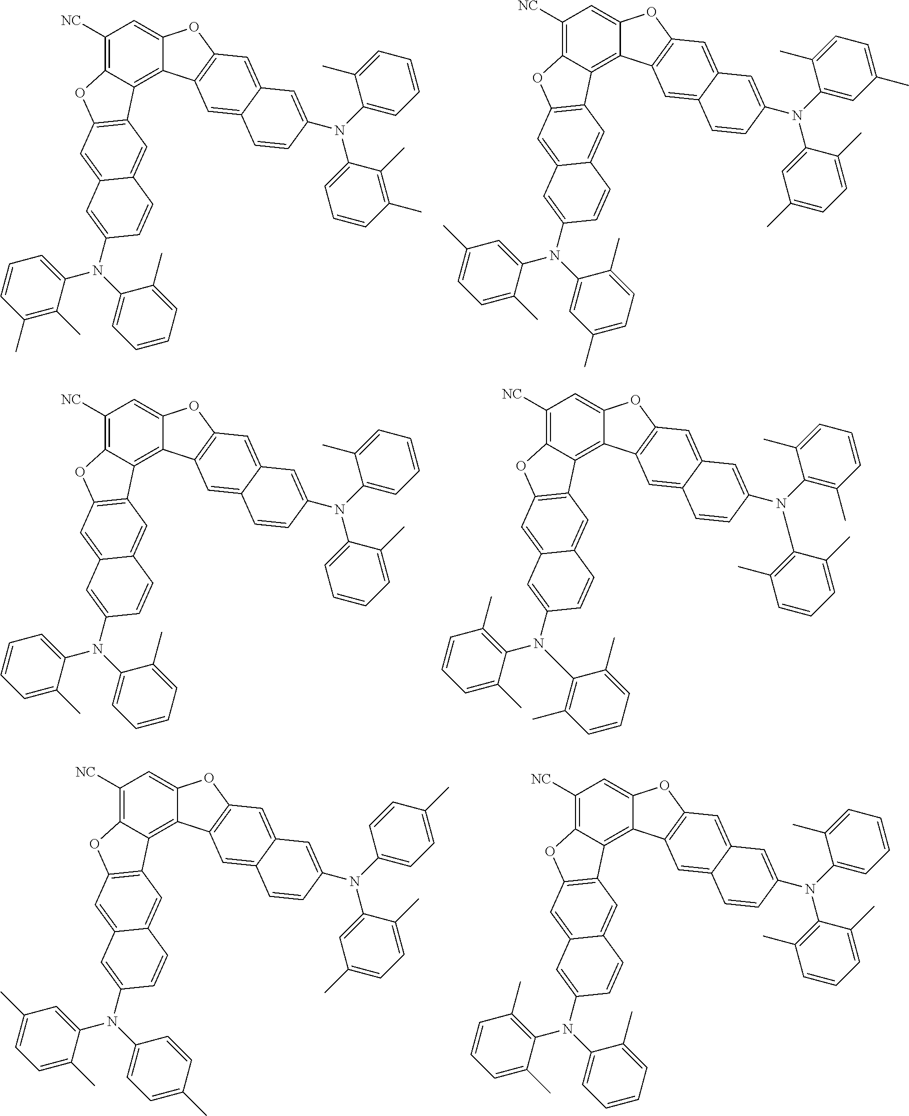

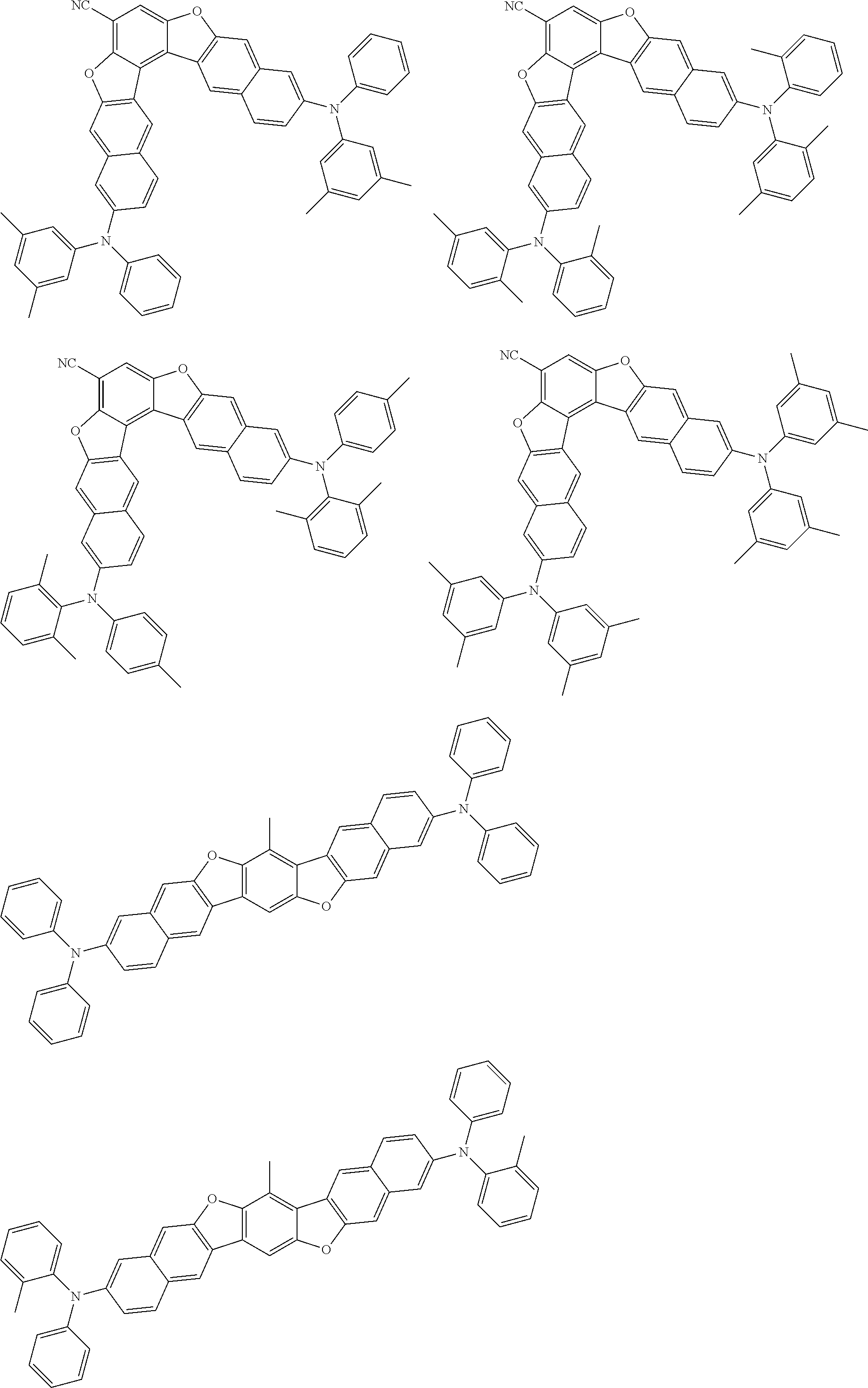

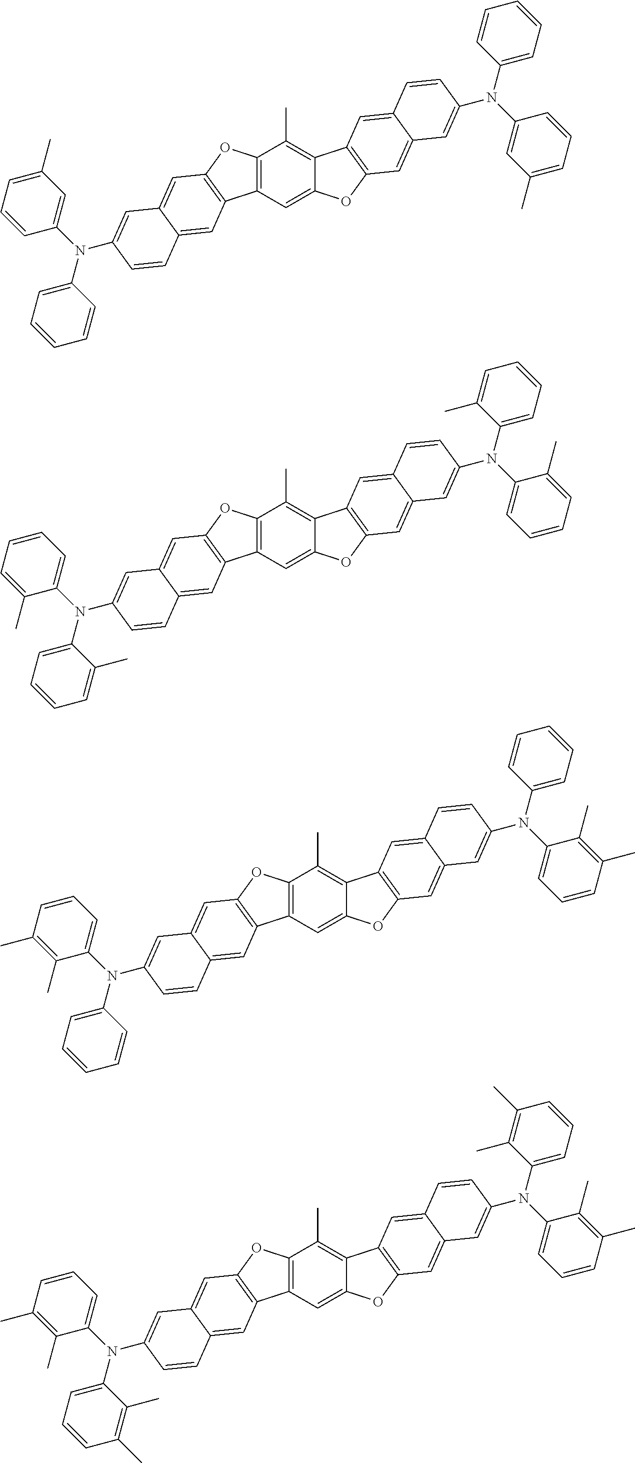

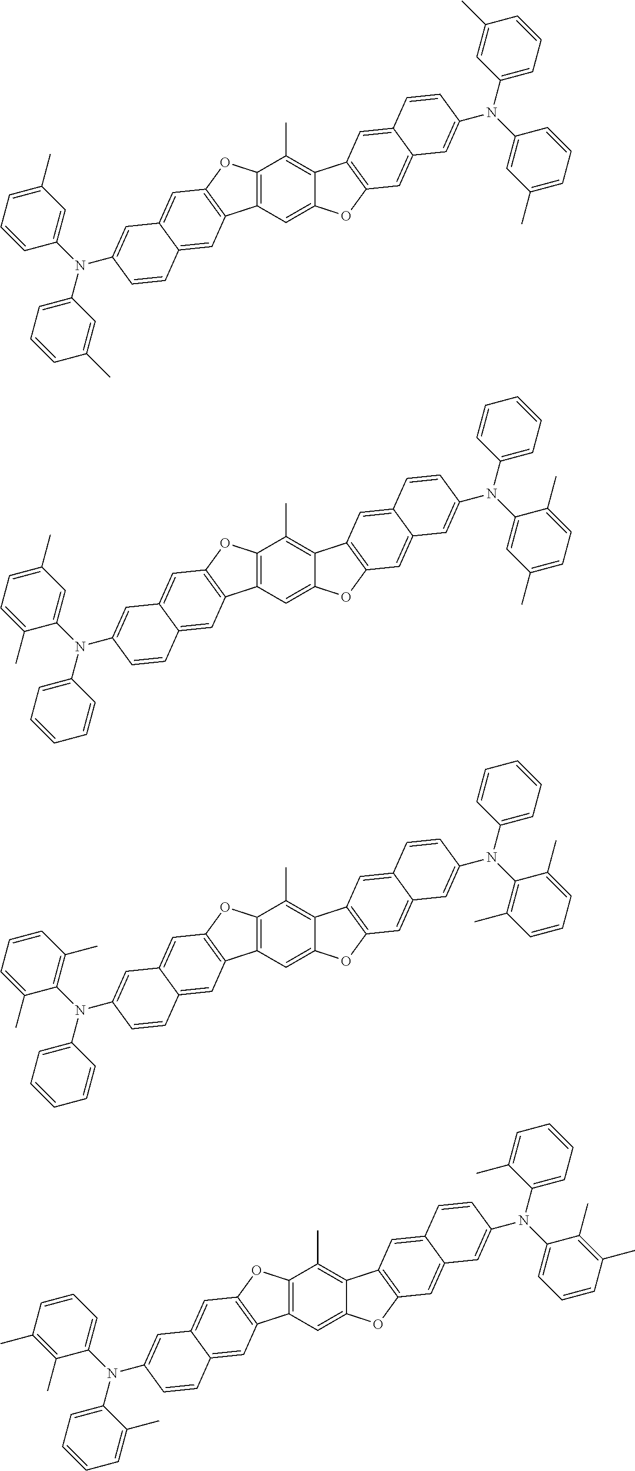

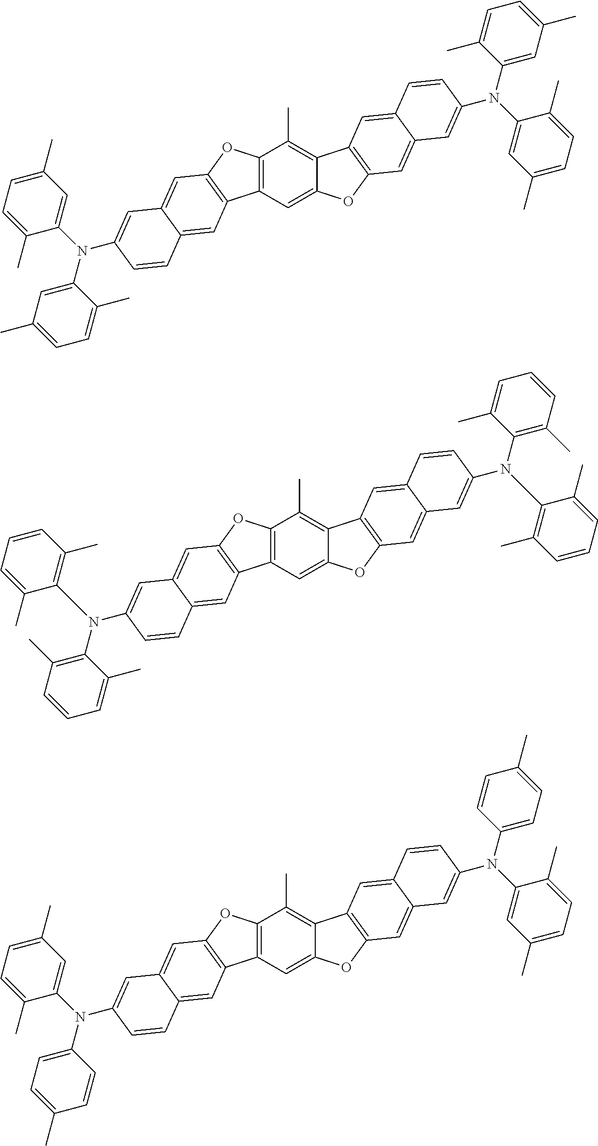

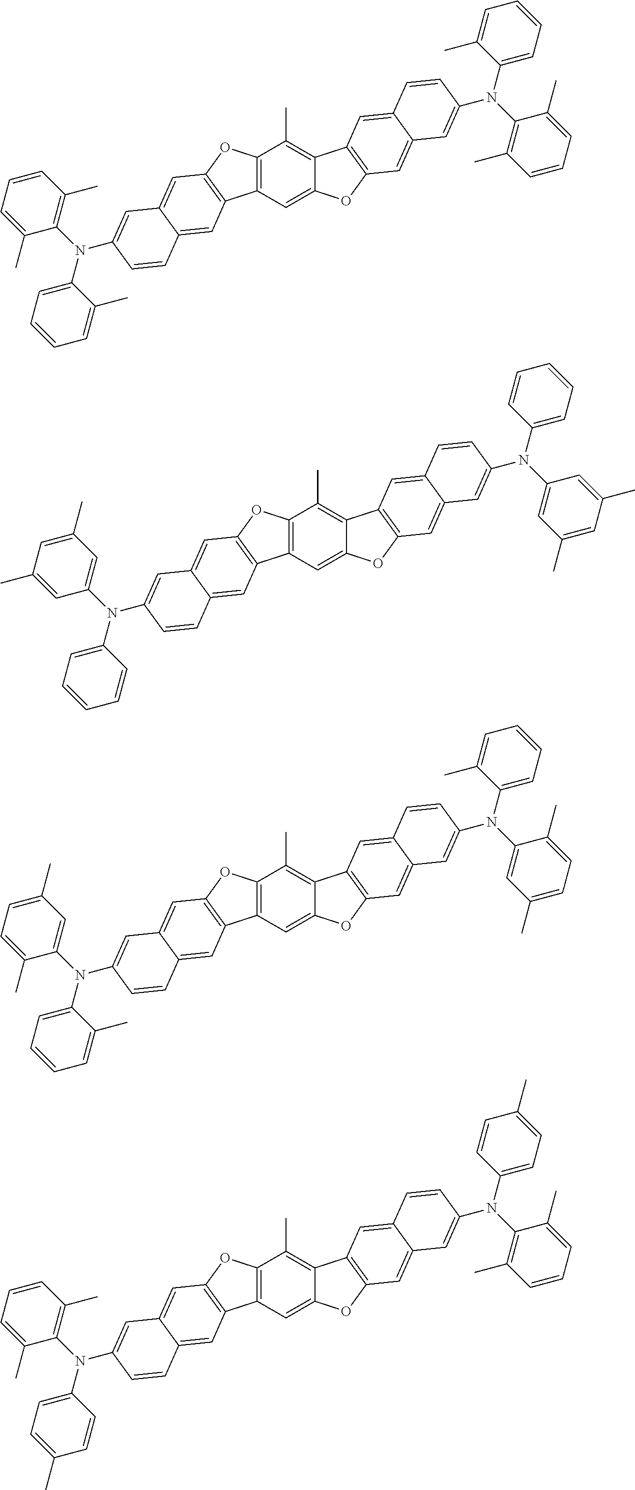

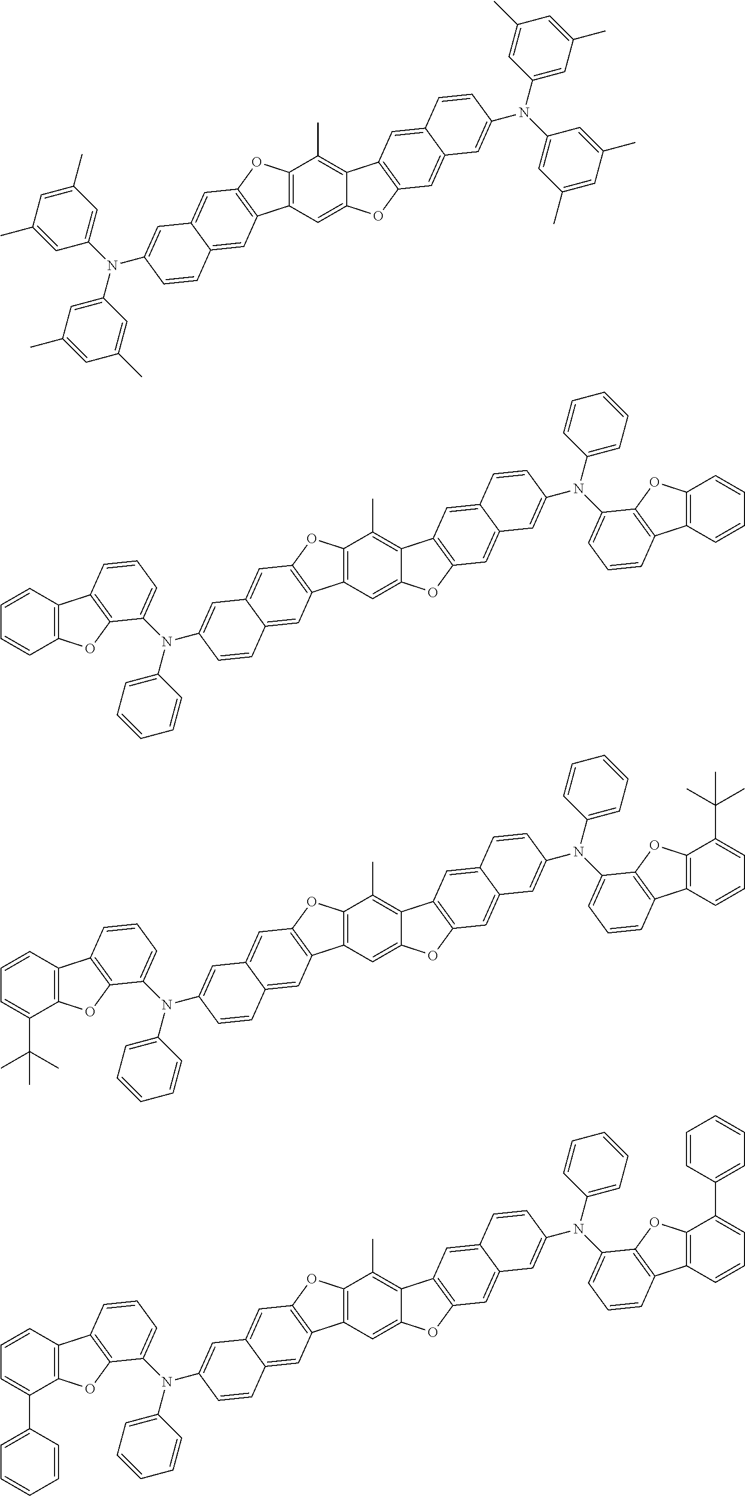

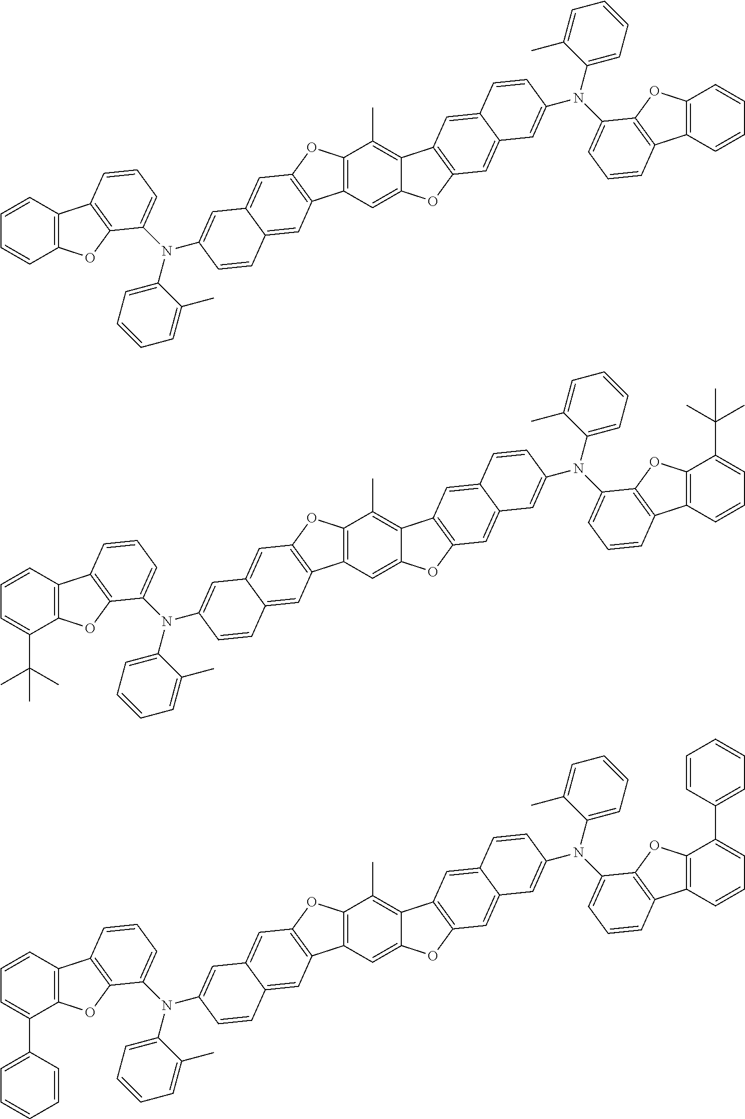 [SMILES: Cc1ccccc1N(c1ccc2cc3c(cc2c1)oc1c(C)c2c(cc13)oc1cc3cc(N(c4ccccc4C)c4cccc5c4oc4ccccc45)ccc3cc12)c1cccc2c1oc1ccccc12 ; Cc1ccccc1N(c1ccc2cc3c(cc2c1)oc1c(C)c2c(cc13)oc1cc3cc(N(c4ccccc4C)c4cccc5c4oc4c(C(C)(C)C)cccc45)ccc3cc12)c1cccc2c1oc1c(C(C)(C)C)cccc12 ; Cc1ccccc1N(c1ccc2cc3c(cc2c1)oc1c(C)c2c(cc13)oc1cc3cc(N(c4ccccc4C)c4cccc5c4oc4c(-c6ccccc6)cccc45)ccc3cc12)c1cccc2c1oc1c(-c3ccccc3)cccc12]

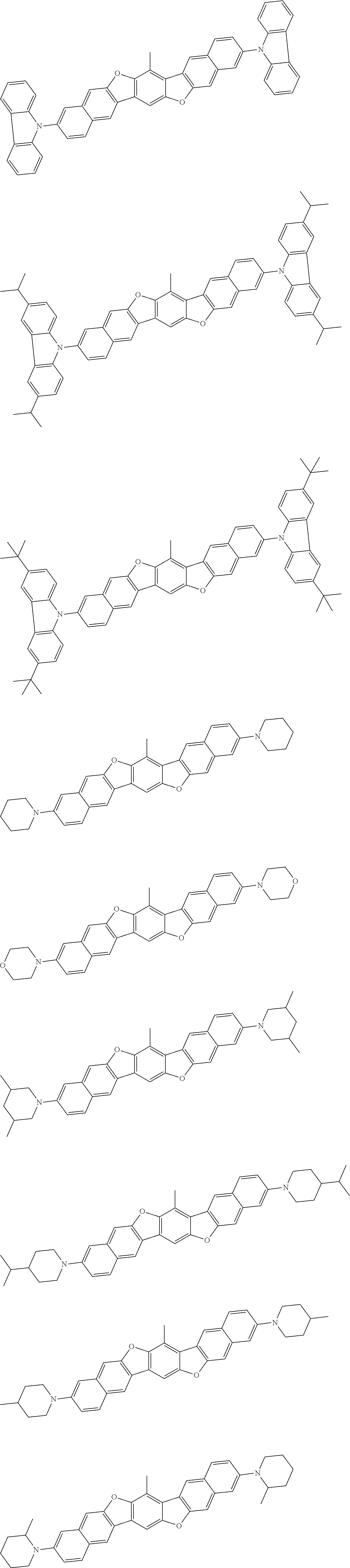

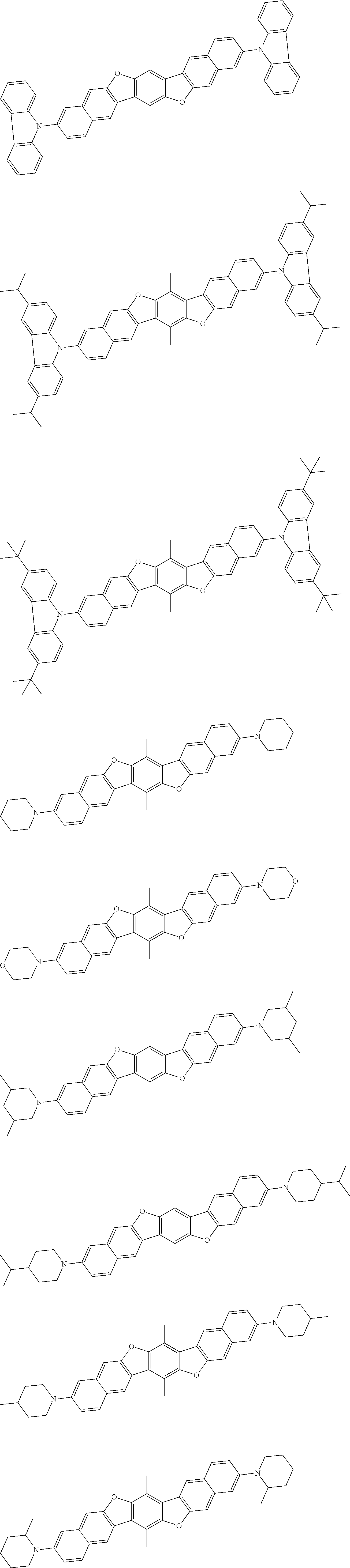

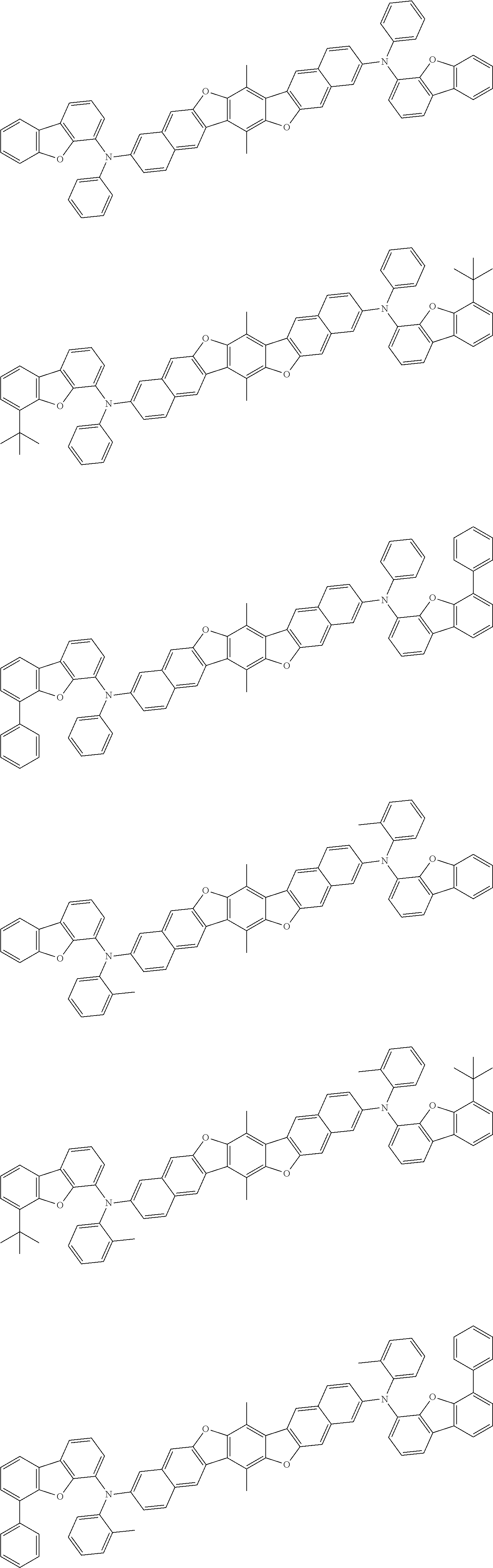

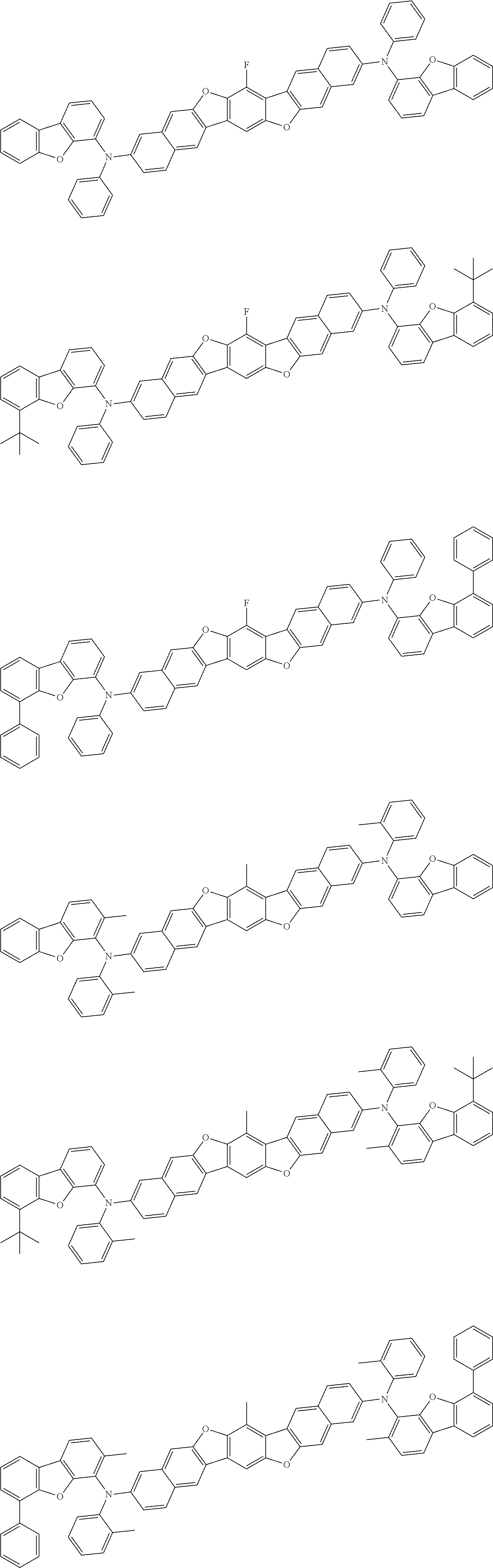

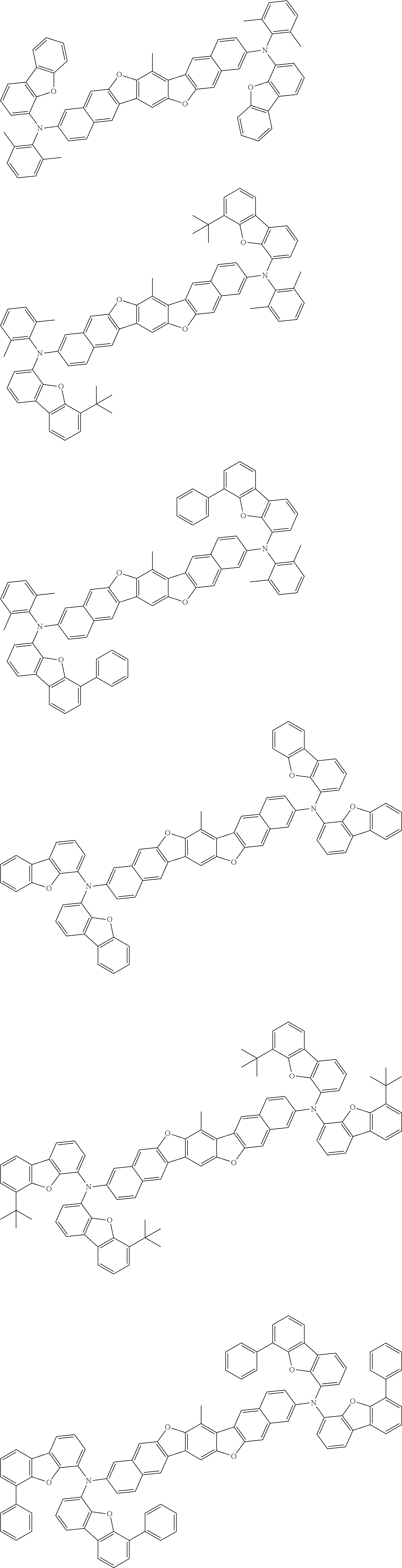

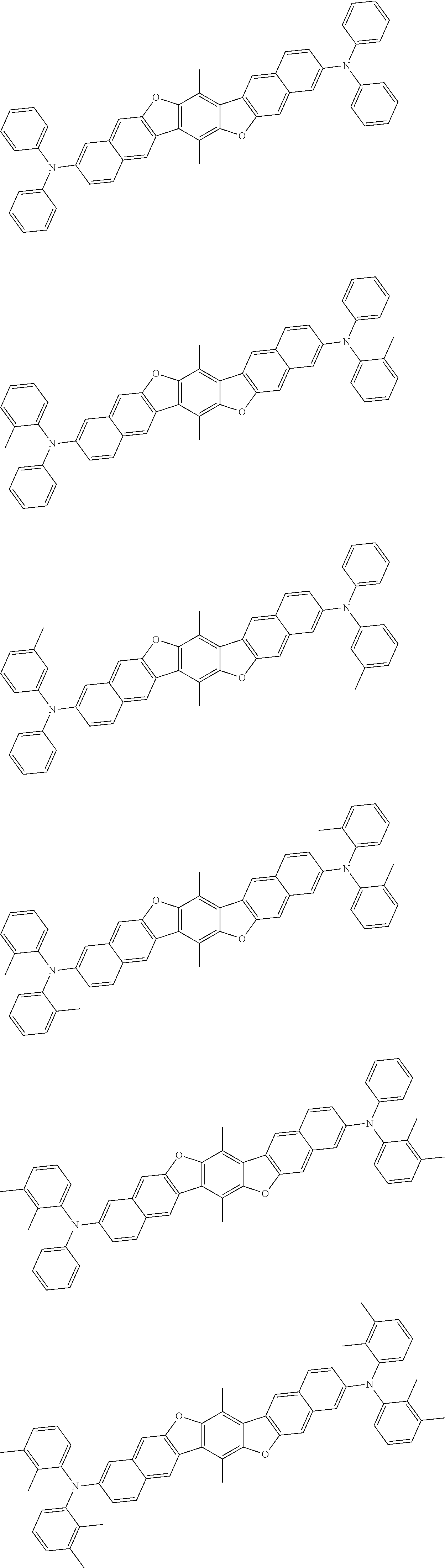

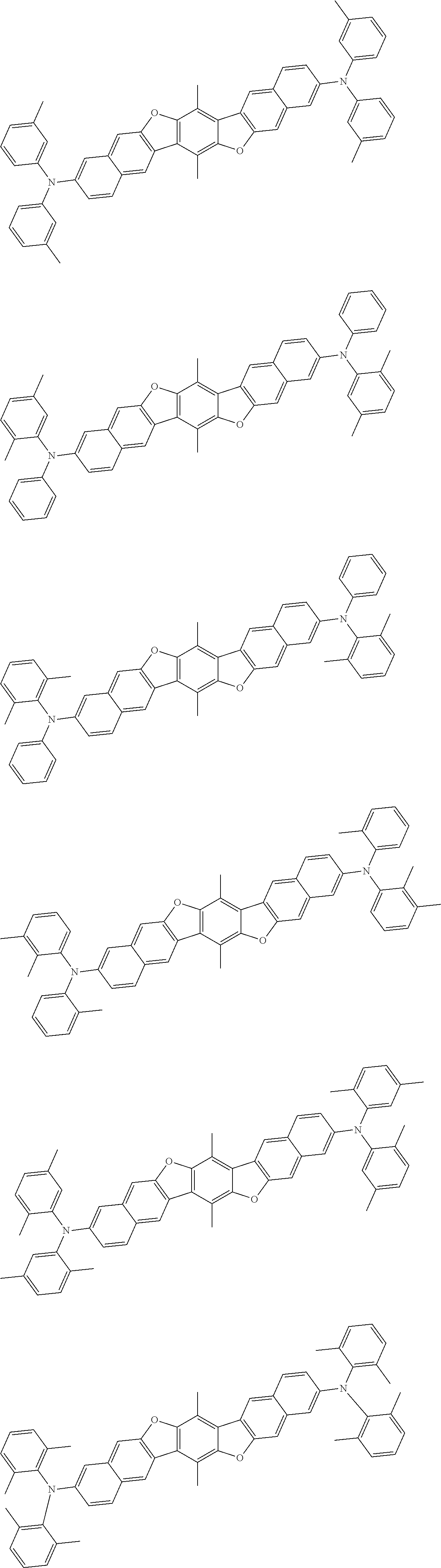

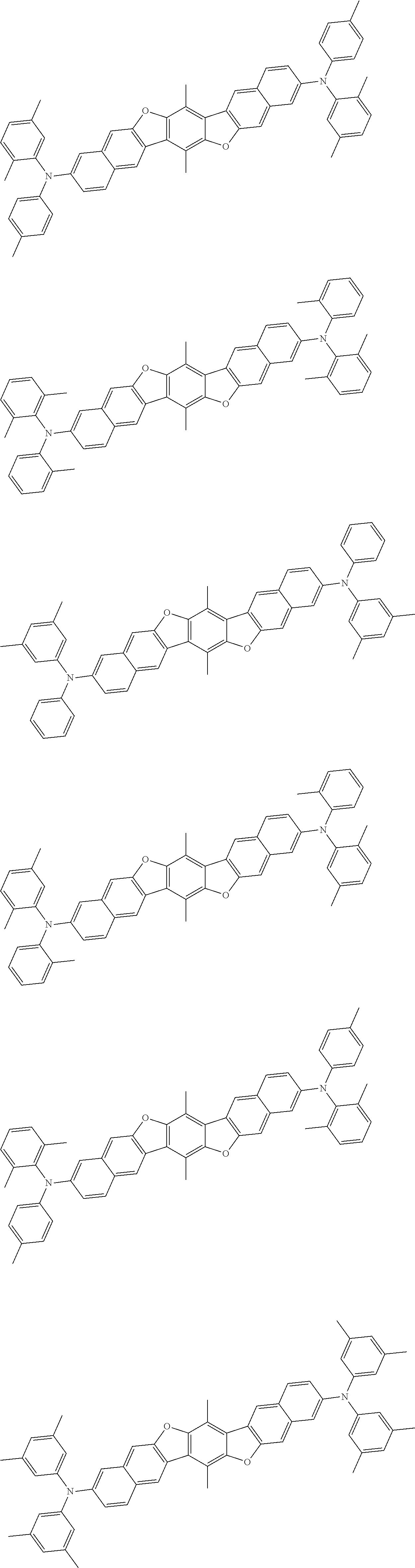

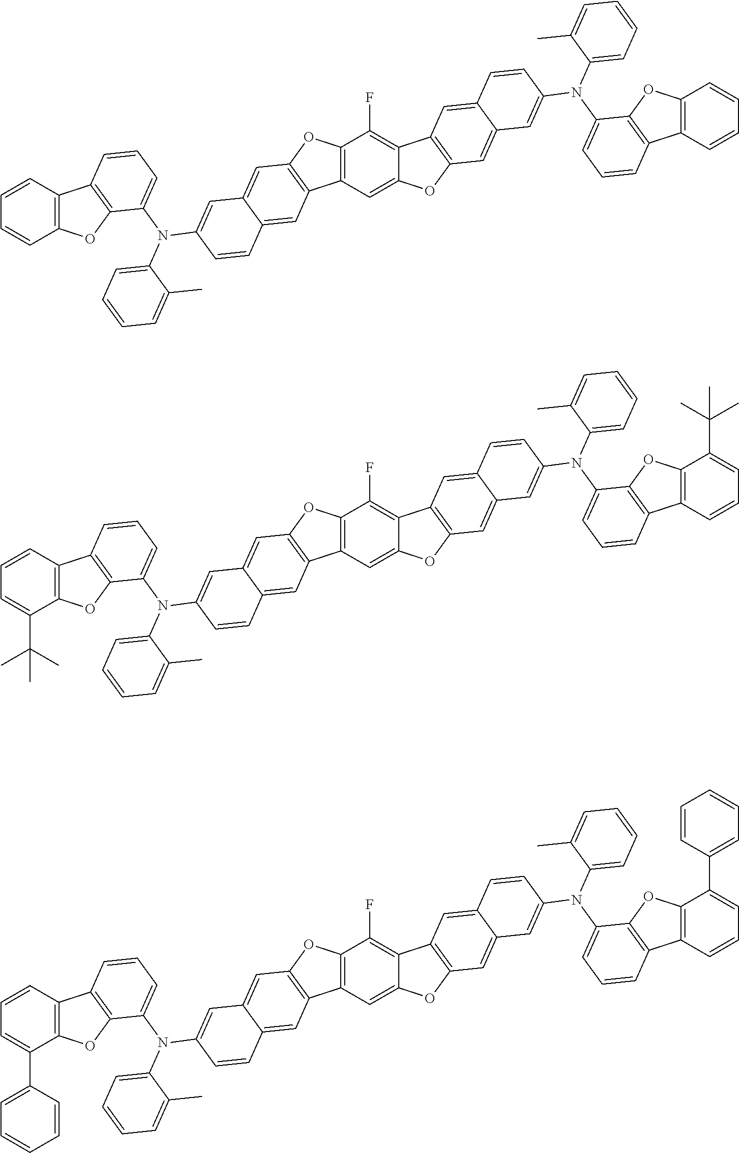 [SMILES: Cc1ccccc1N(c1ccc2cc3c(cc2c1)oc1c(F)c2c(cc13)oc1cc3cc(N(c4ccccc4C)c4cccc5c4oc4ccccc45)ccc3cc12)c1cccc2c1oc1ccccc12 ; Cc1ccccc1N(c1ccc2cc3c(cc2c1)oc1c(F)c2c(cc13)oc1cc3cc(N(c4ccccc4C)c4cccc5c4oc4c(C(C)(C)C)cccc45)ccc3cc12)c1cccc2c1oc1c(C(C)(C)C)cccc12 ; Cc1ccccc1N(c1ccc2cc3c(cc2c1)oc1c(F)c2c(cc13)oc1cc3cc(N(c4ccccc4C)c4cccc5c4oc4c(-c6ccccc6)cccc45)ccc3cc12)c1cccc2c1oc1c(-c3ccccc3)cccc12]

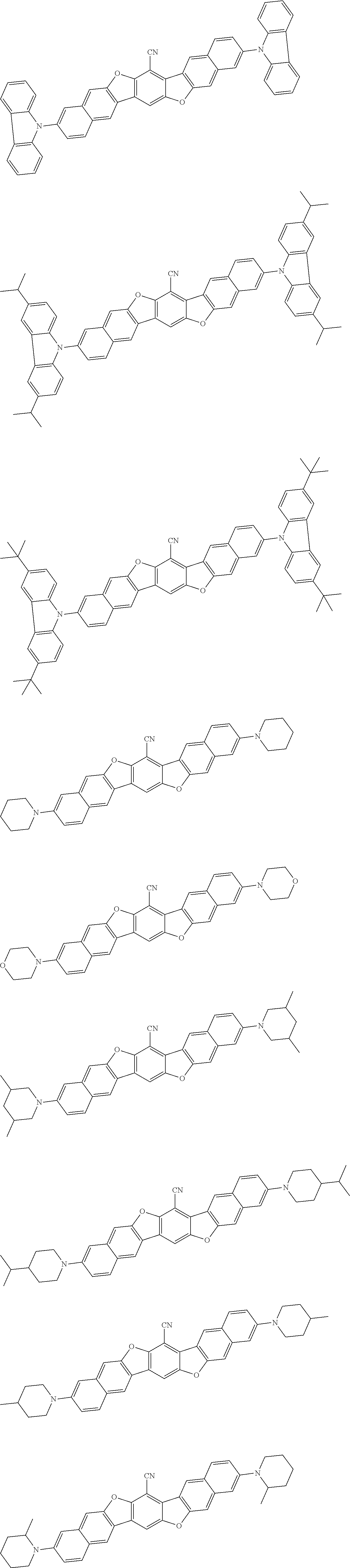

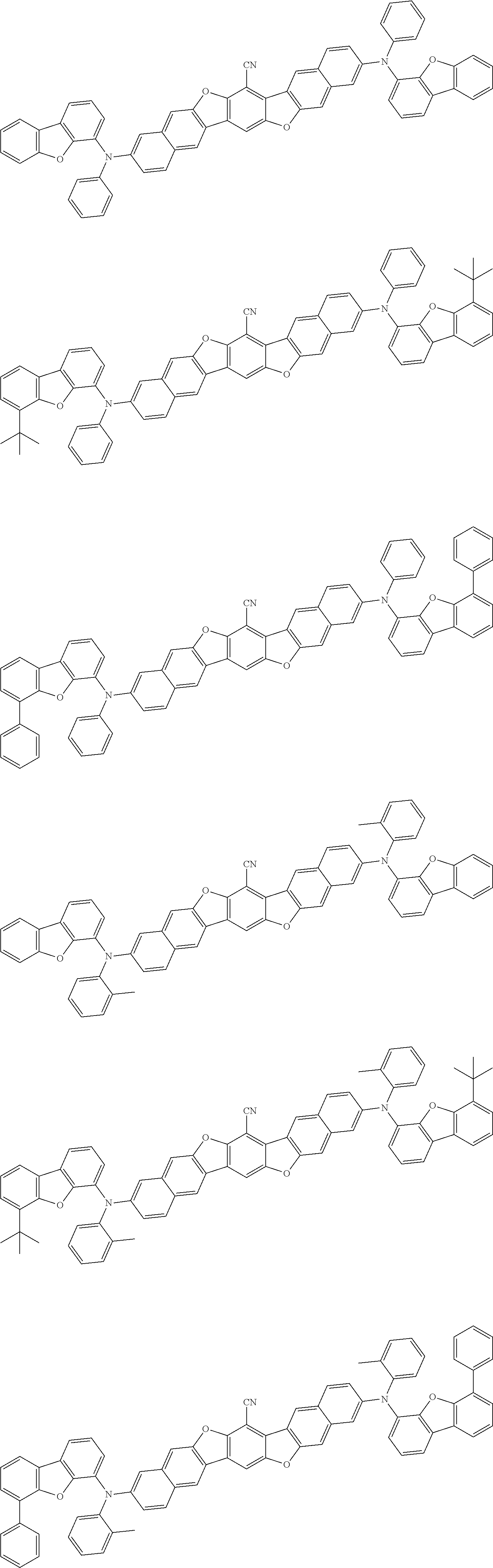

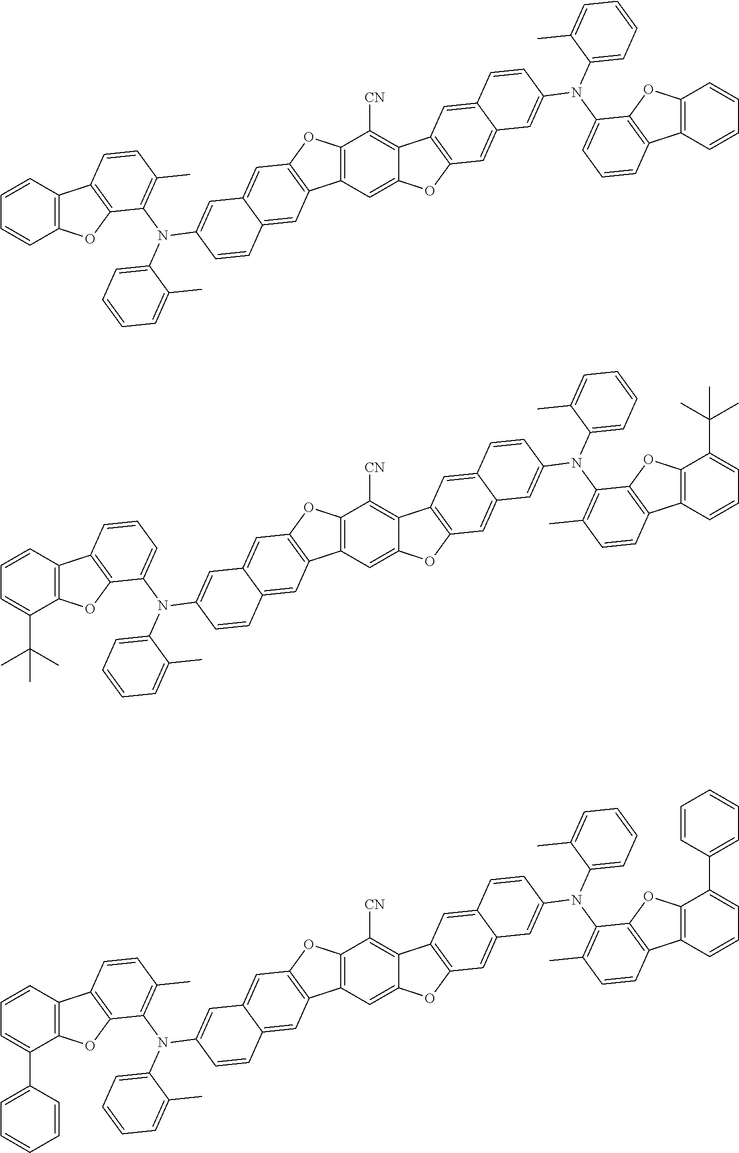 [SMILES: Cc1ccccc1N(c1ccc2cc3c(cc2c1)oc1cc2c(oc4cc5cc(N(c6ccccc6C)c6c(C)ccc7c6oc6ccccc67)ccc5cc42)c(C#N)c13)c1cccc2c1oc1ccccc12 ; Cc1ccccc1N(c1ccc2cc3c(cc2c1)oc1c(C#N)c2c(cc13)oc1cc3cc(N(c4ccccc4C)c4c(C)ccc5c4oc4c(C(C)(C)C)cccc45)ccc3cc12)c1cccc2c1oc1c(C(C)(C)C)cccc12 ; Cc1ccccc1N(c1ccc2cc3c(cc2c1)oc1c(C#N)c2c(cc13)oc1cc3cc(N(c4ccccc4C)c4c(C)ccc5c4oc4c(-c6ccccc6)cccc45)ccc3cc12)c1c(C)ccc2c1oc1c(-c3ccccc3)cccc12]

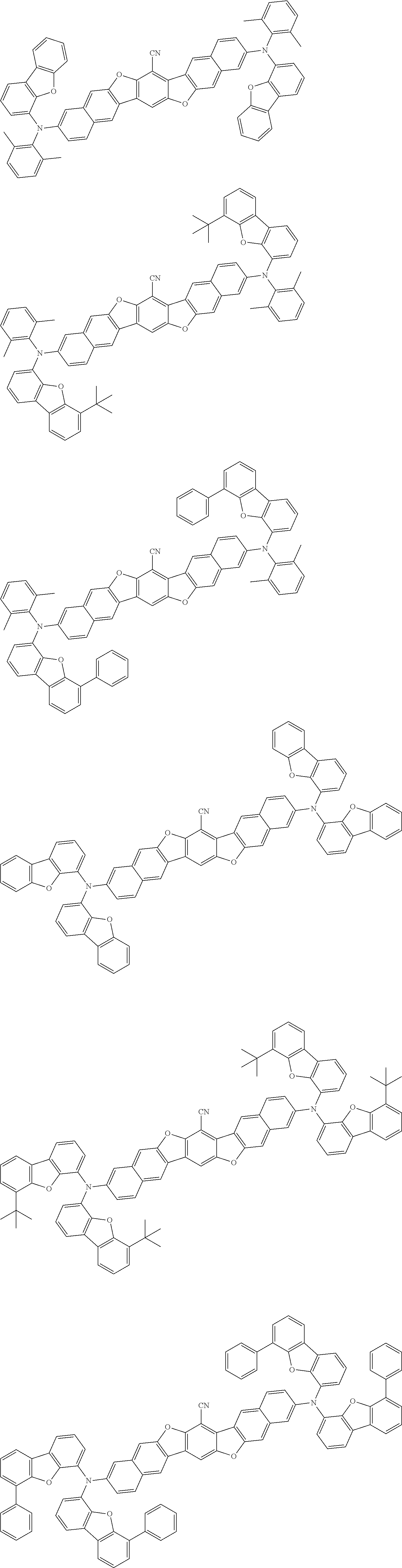

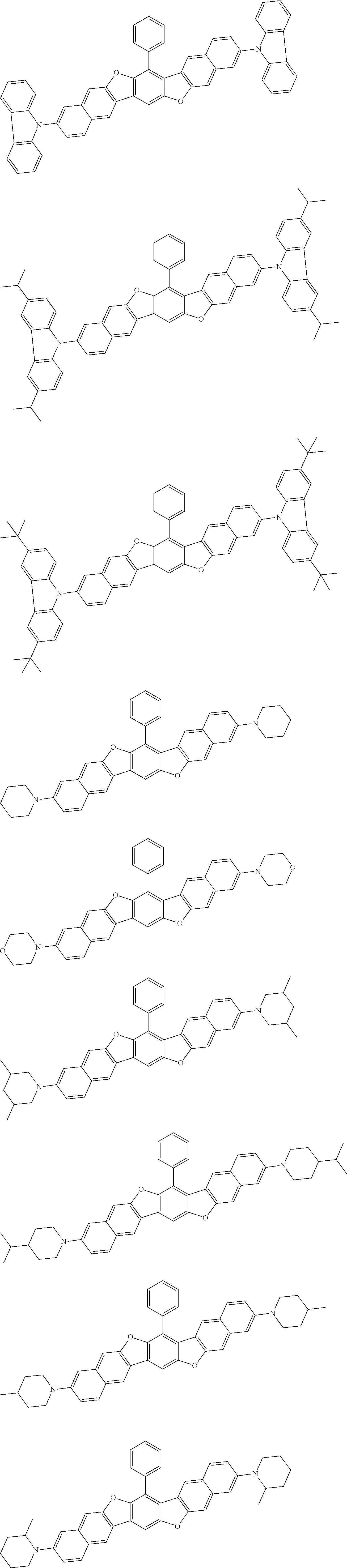

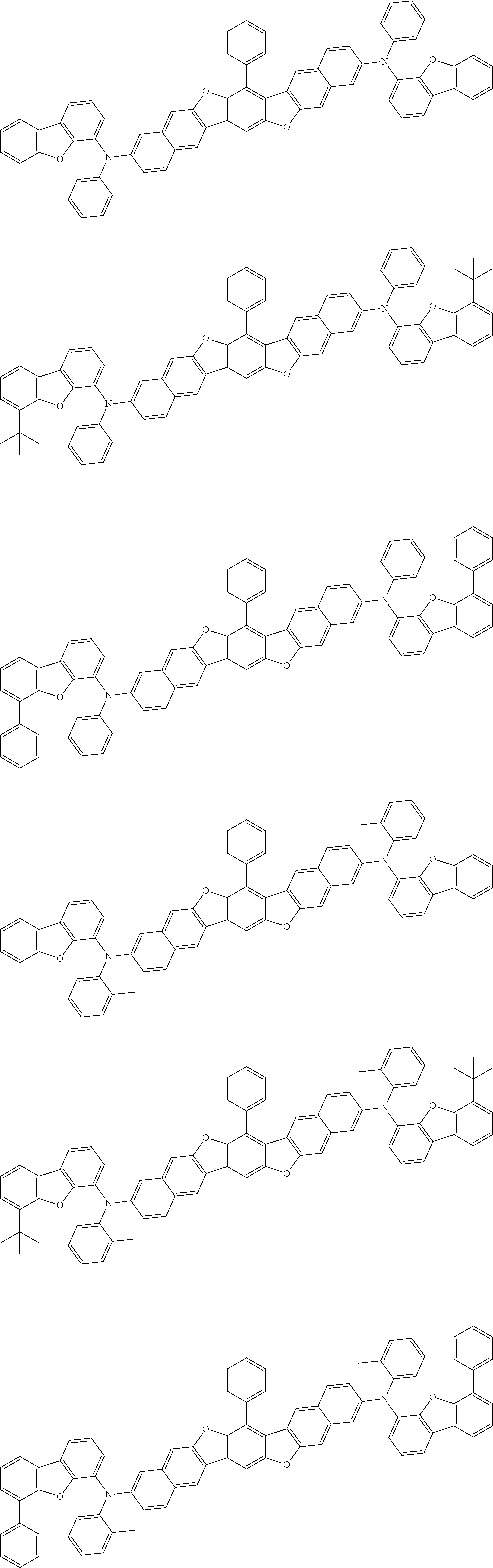

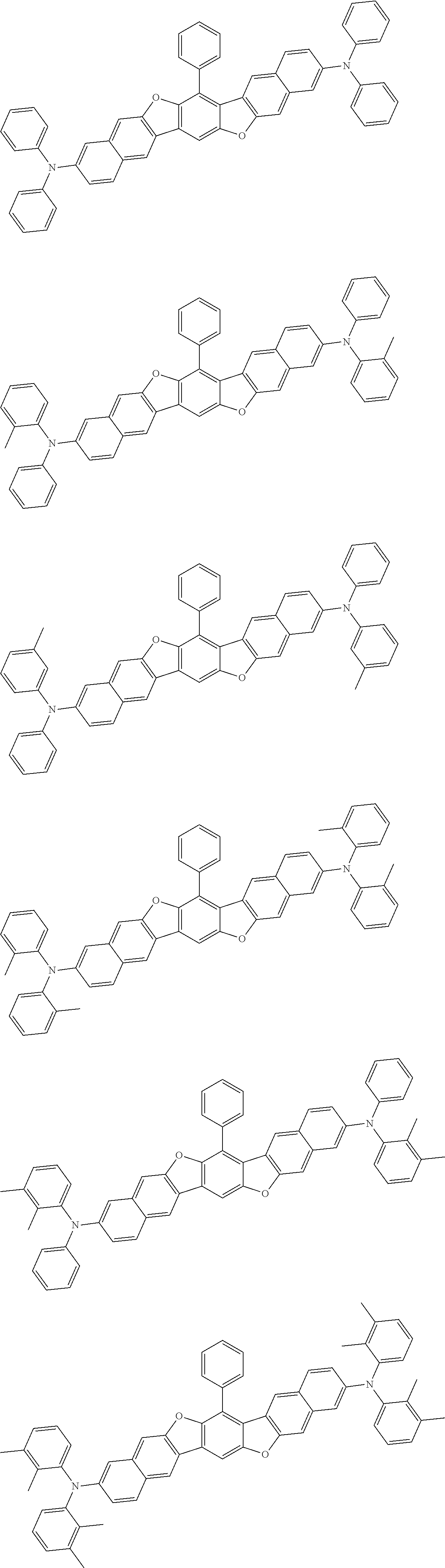

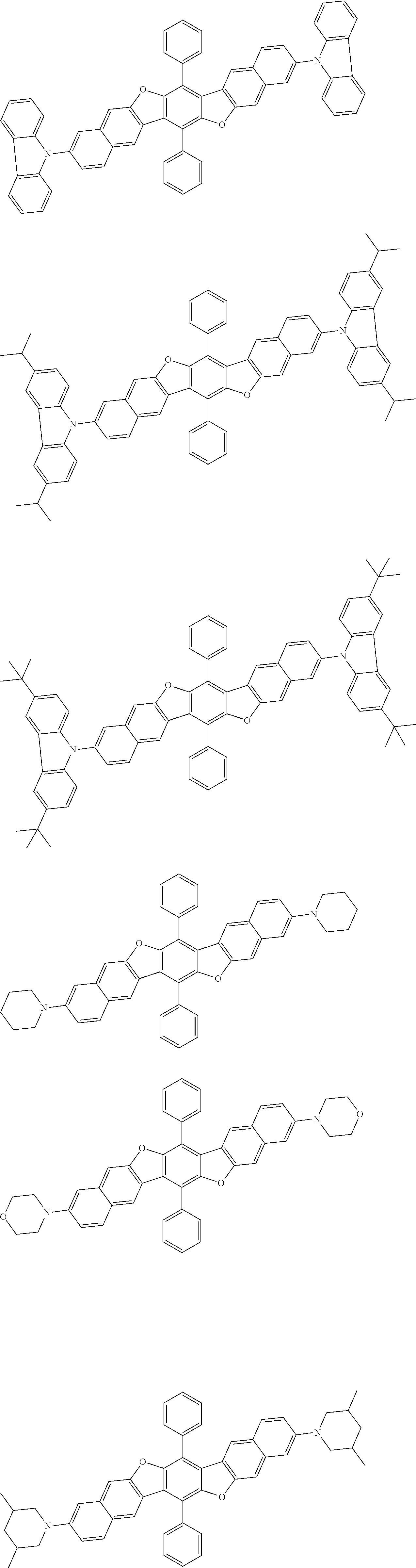

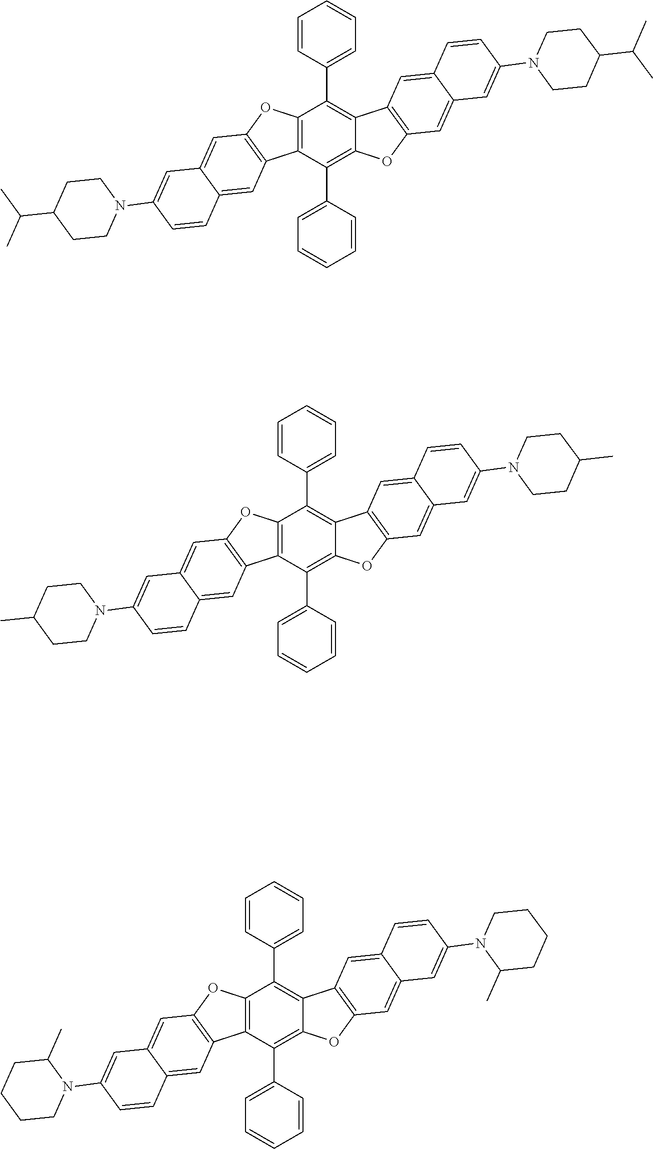

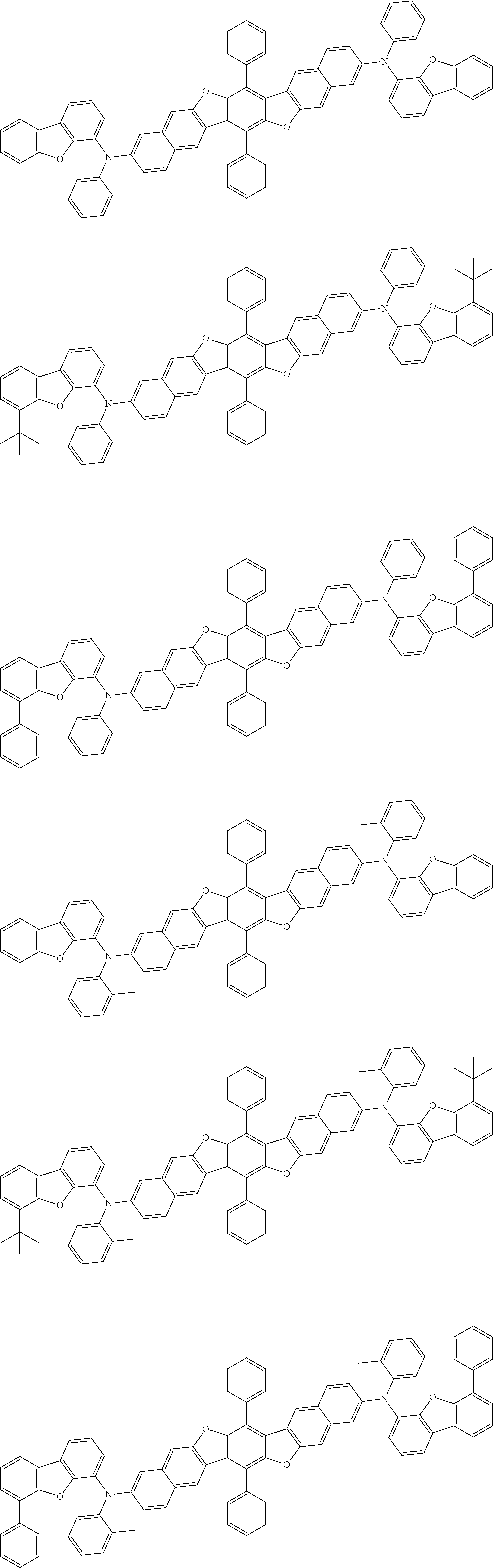

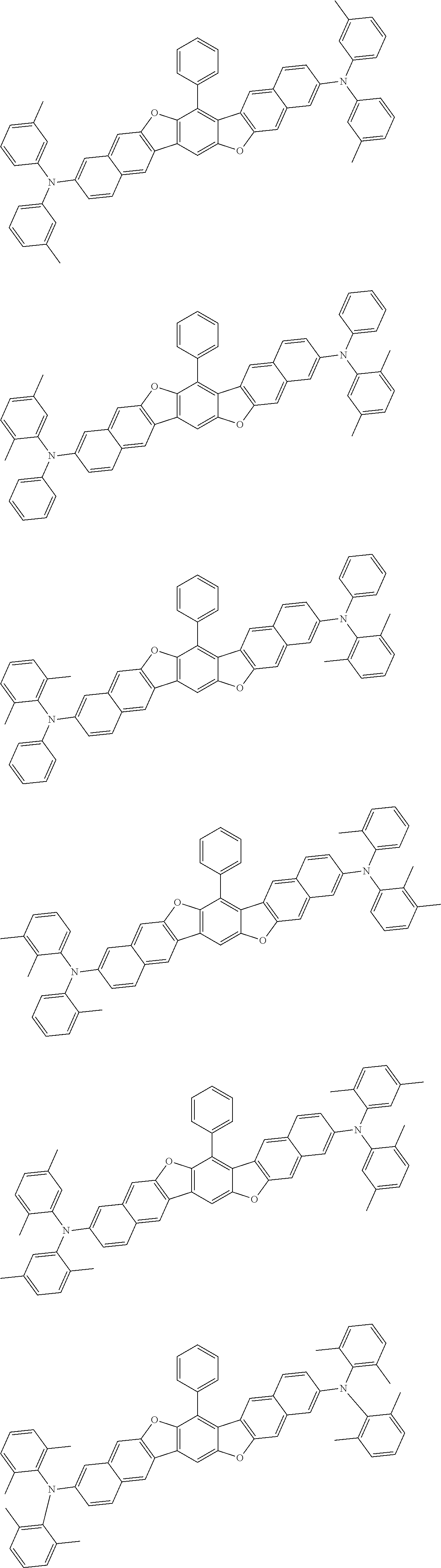

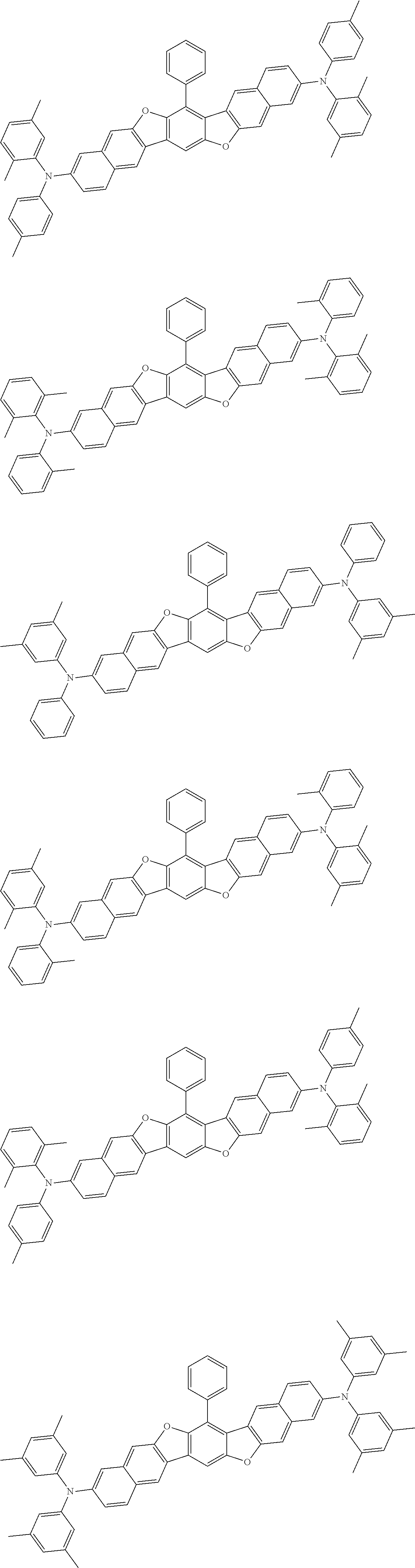

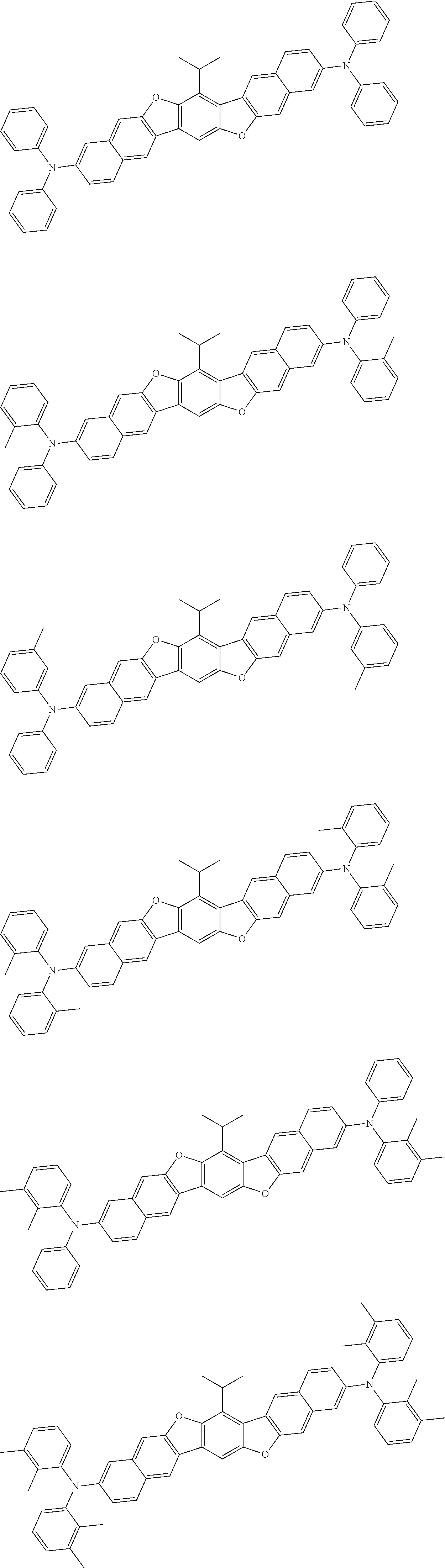

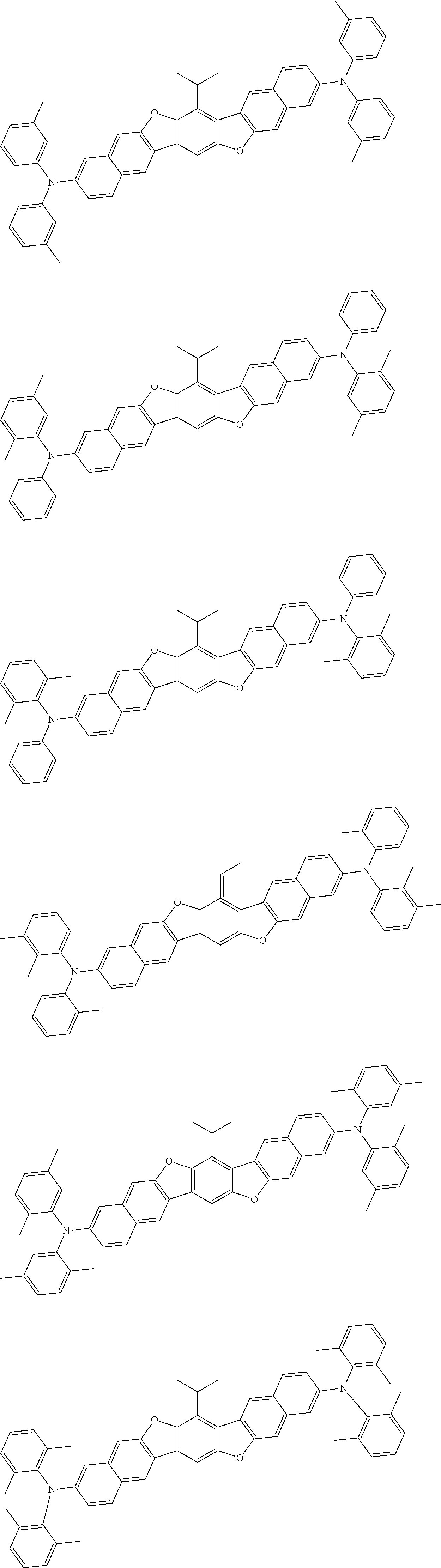

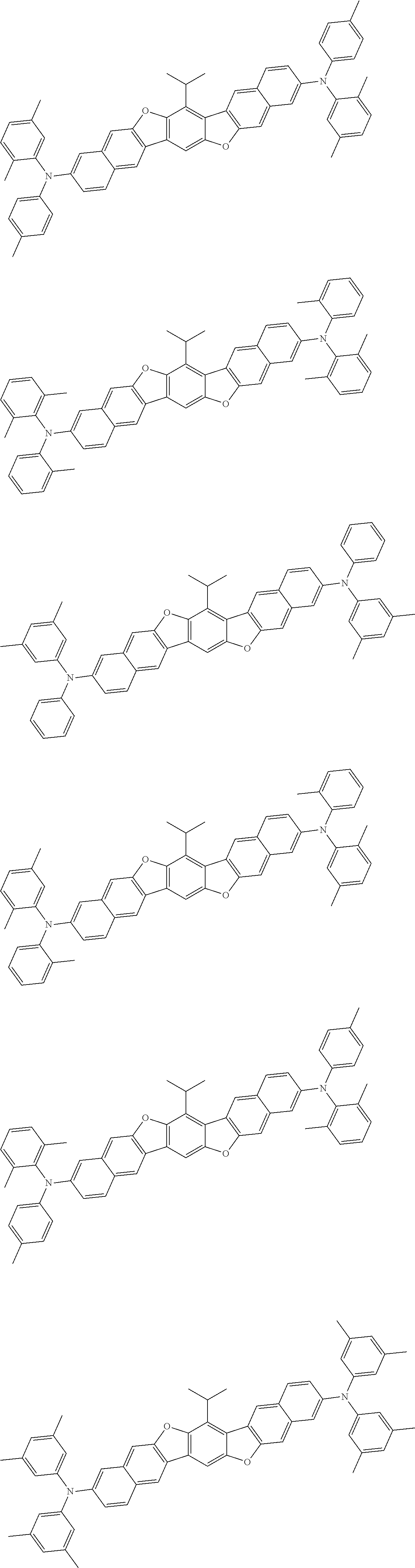

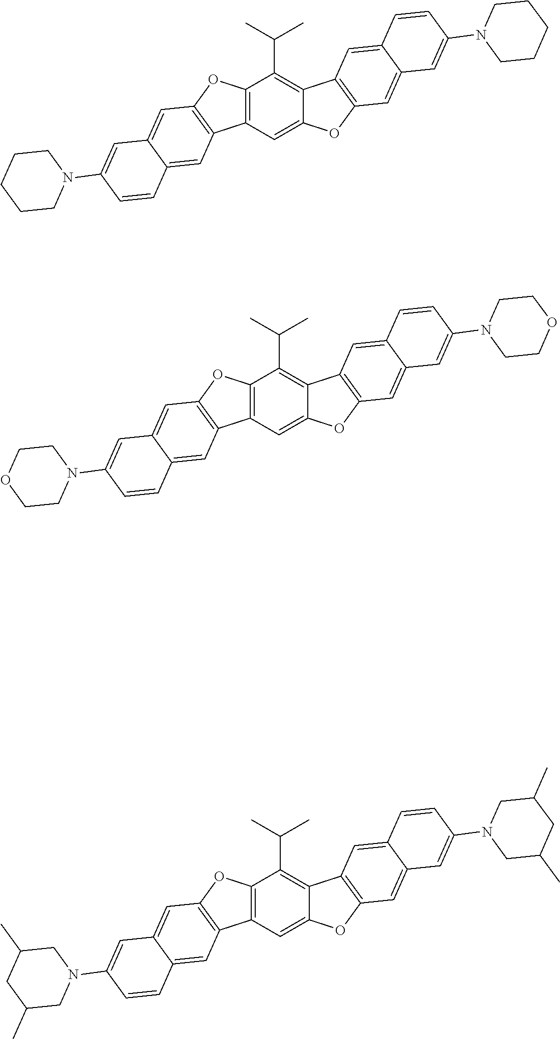

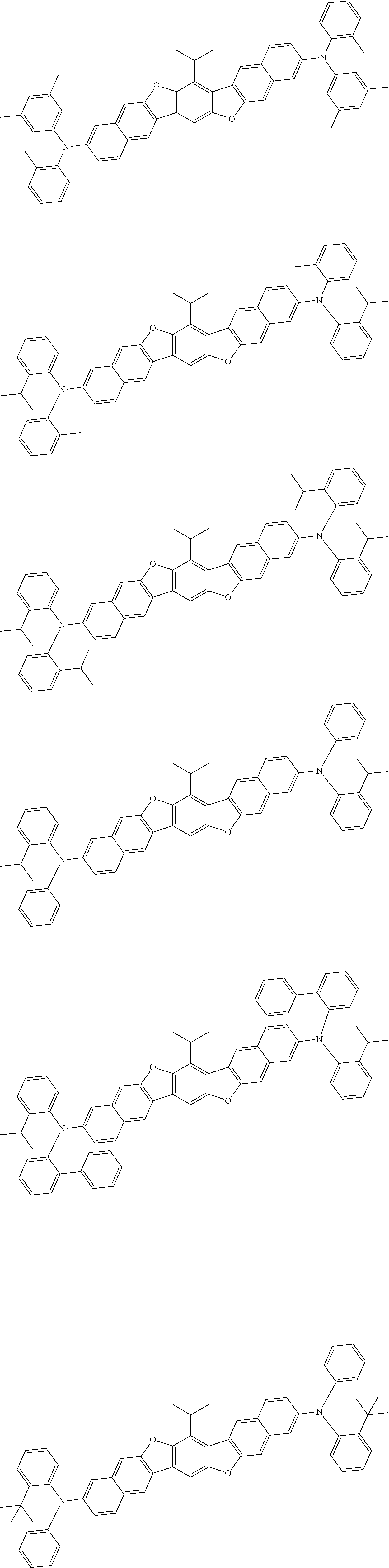

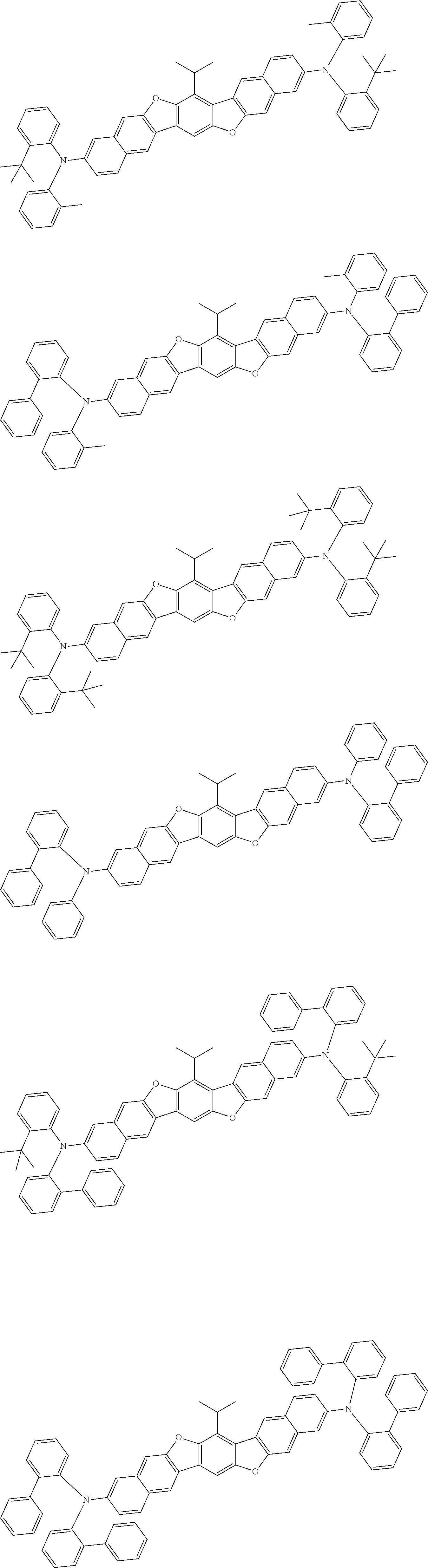

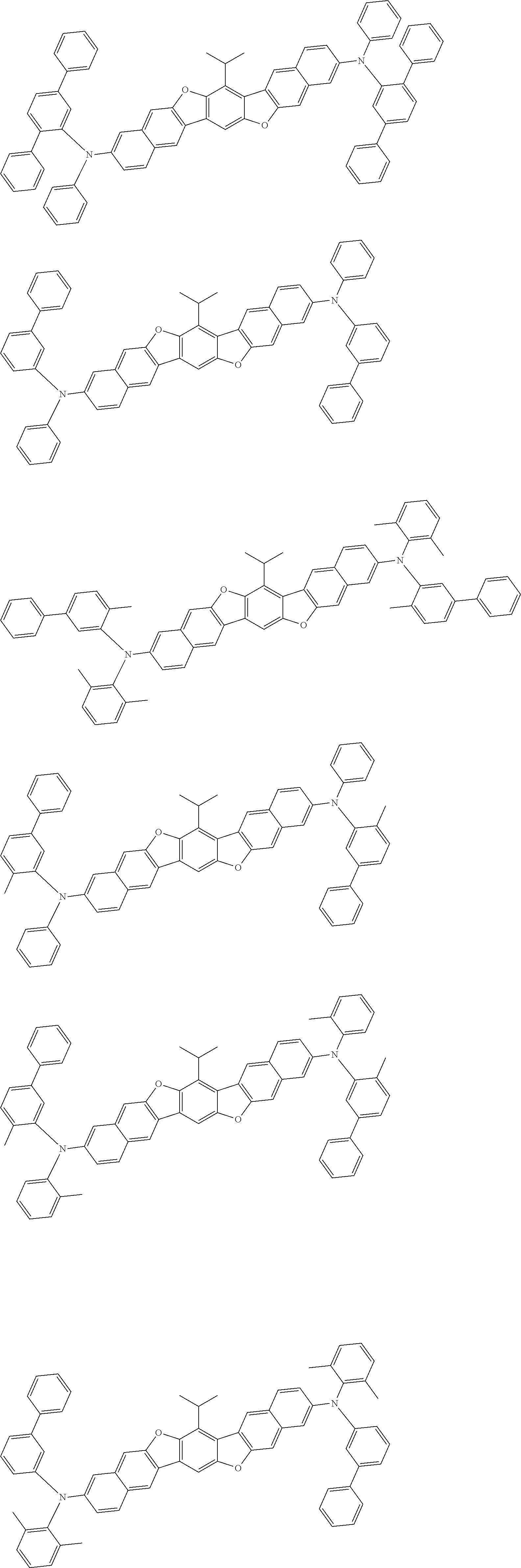

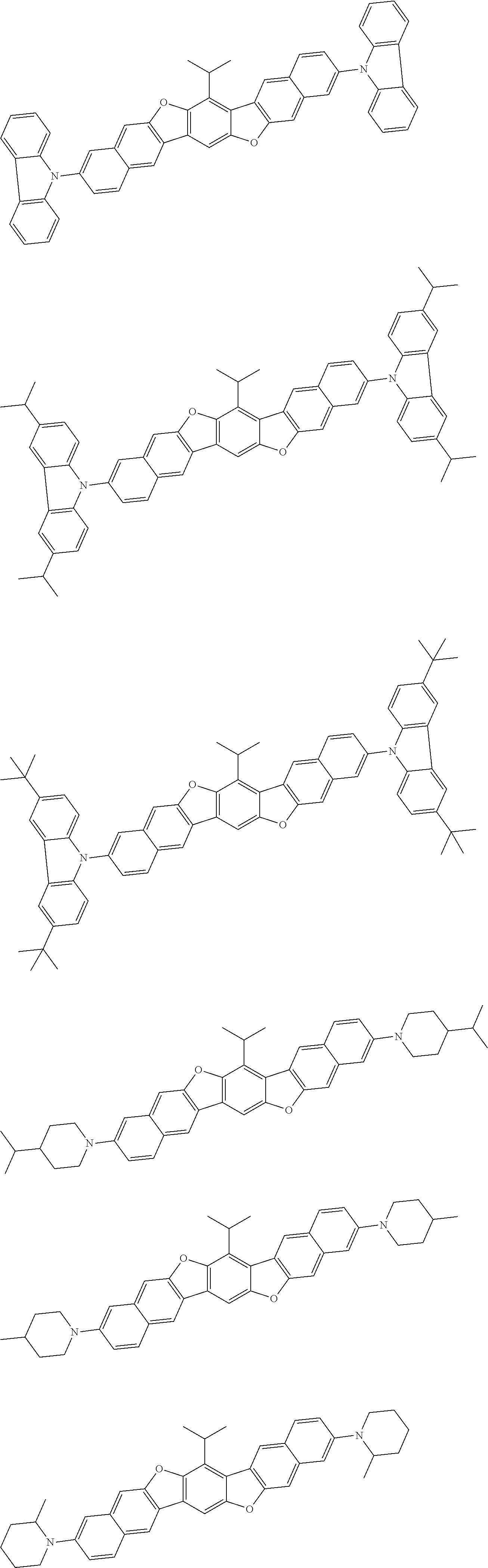

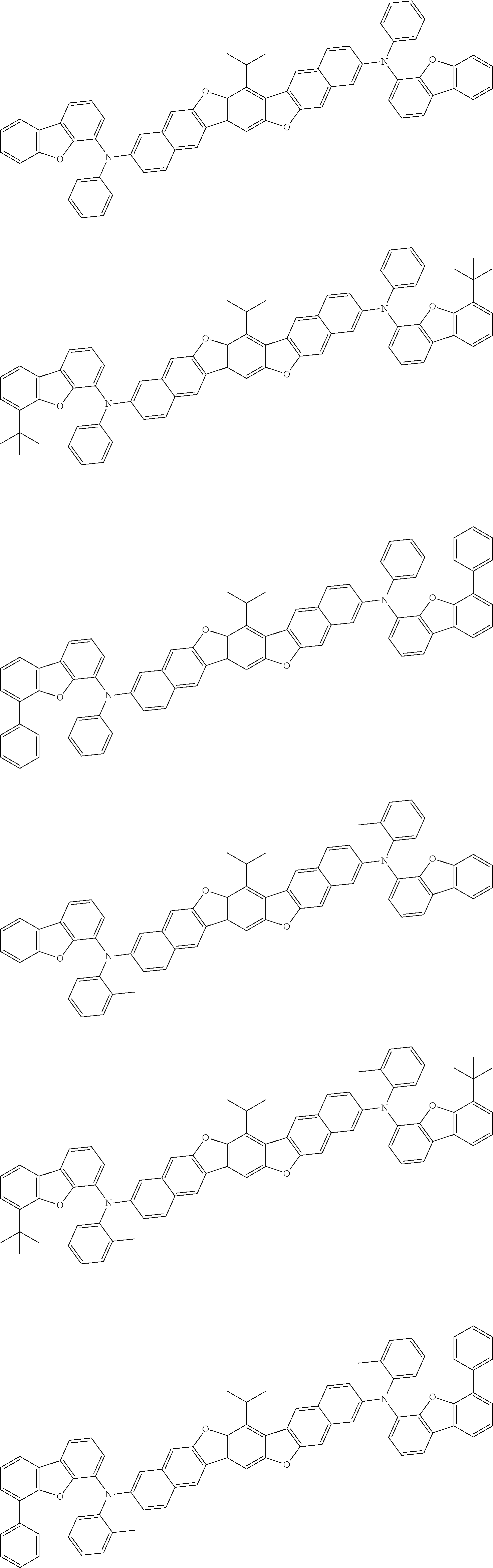

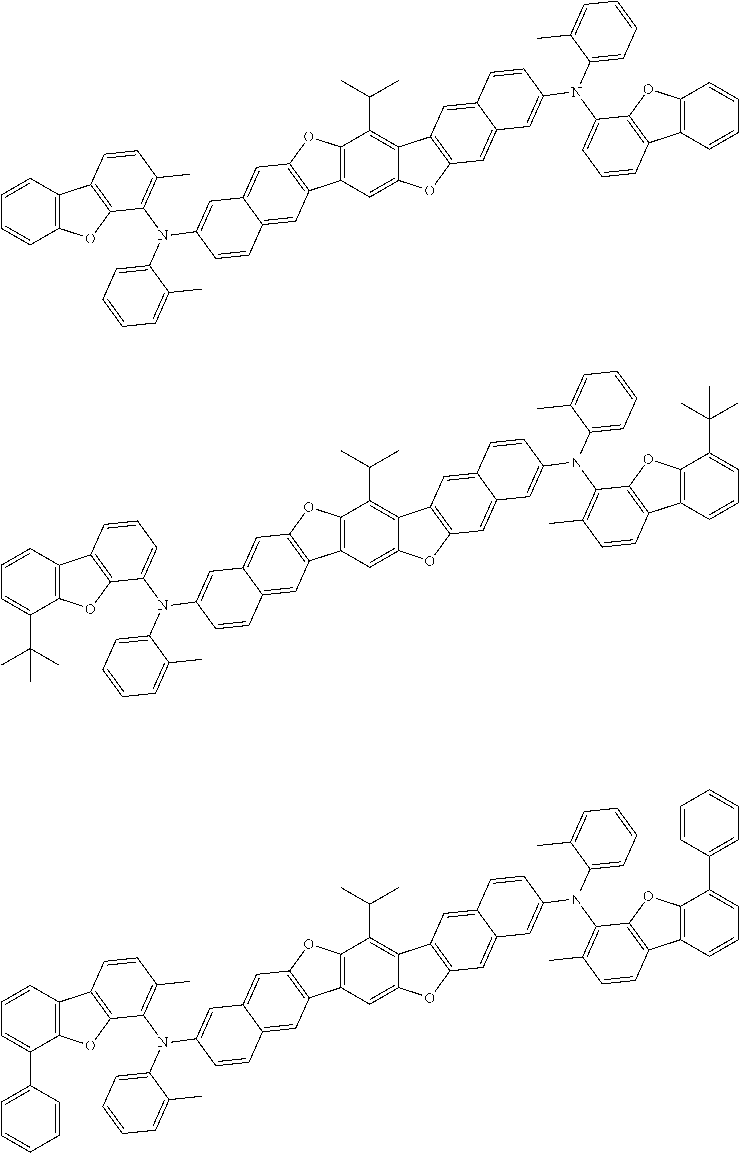 [SMILES: Cc1ccccc1N(c1ccc2cc3c(cc2c1)oc1cc2c(oc4cc5cc(N(c6ccccc6C)c6c(C)ccc7c6oc6ccccc67)ccc5cc42)c(C(C)C)c13)c1cccc2c1oc1ccccc12 ; Cc1ccccc1N(c1ccc2cc3c(cc2c1)oc1c(C(C)C)c2c(cc13)oc1cc3cc(N(c4ccccc4C)c4c(C)ccc5c4oc4c(C(C)(C)C)cccc45)ccc3cc12)c1cccc2c1oc1c(C(C)(C)C)cccc12 ; Cc1ccccc1N(c1ccc2cc3c(cc2c1)oc1c(C(C)C)c2c(cc13)oc1cc3cc(N(c4ccccc4C)c4c(C)ccc5c4oc4c(-c6ccccc6)cccc45)ccc3cc12)c1c(C)ccc2c1oc1c(-c3ccccc3)cccc12]

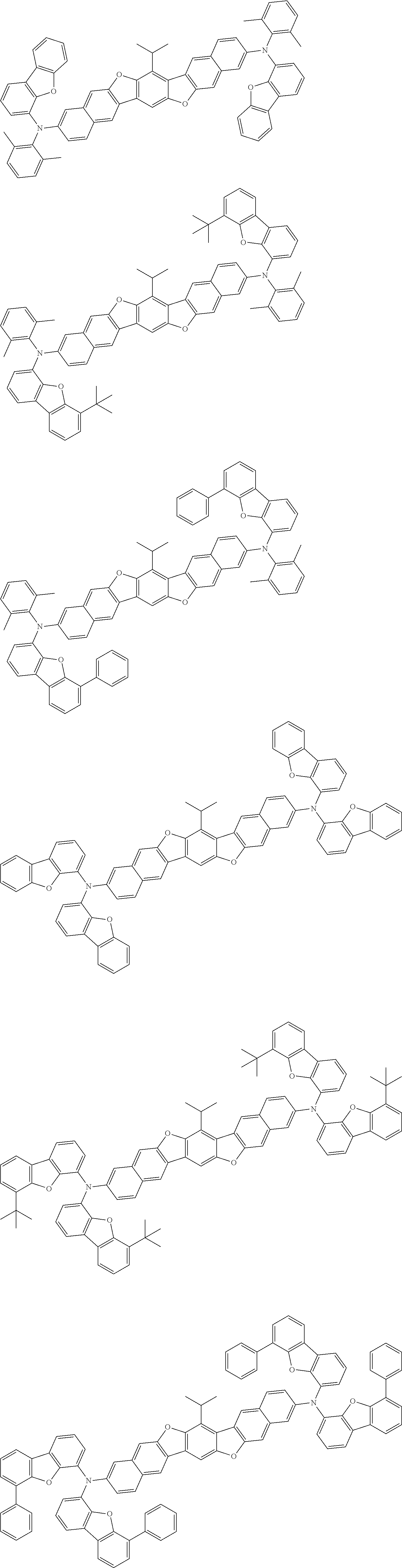

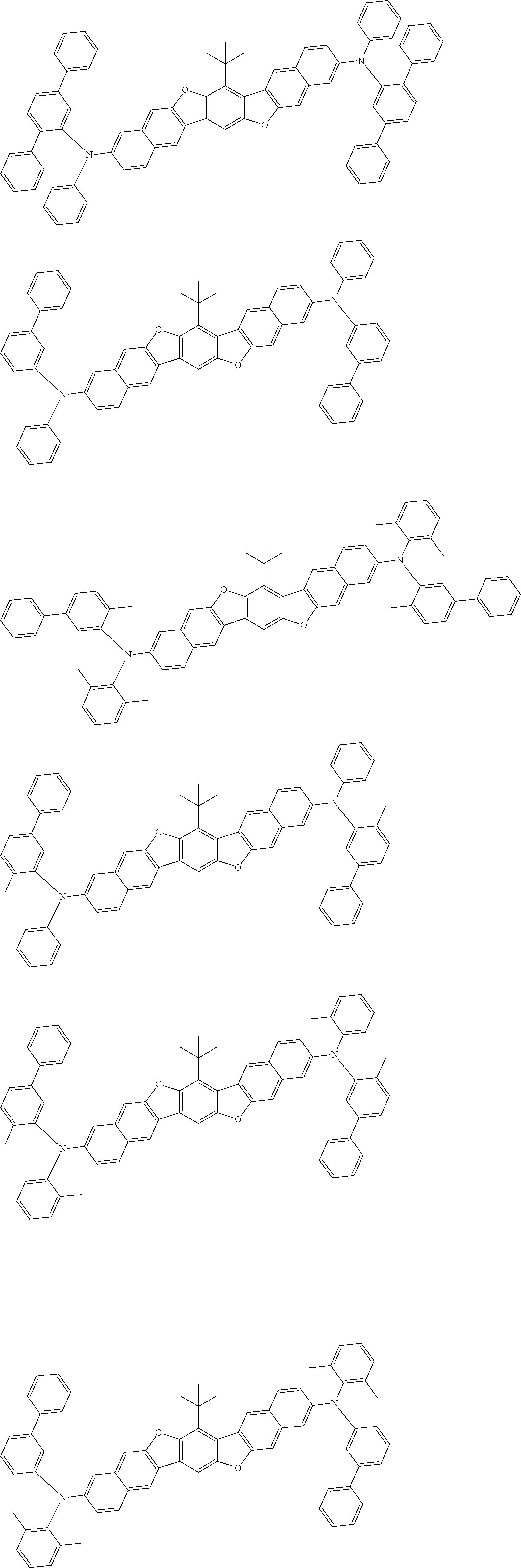

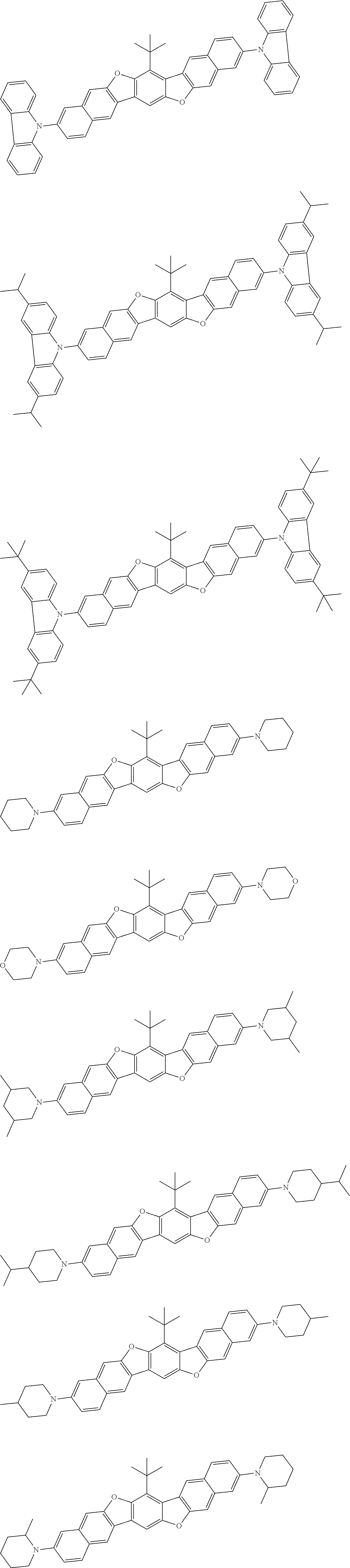

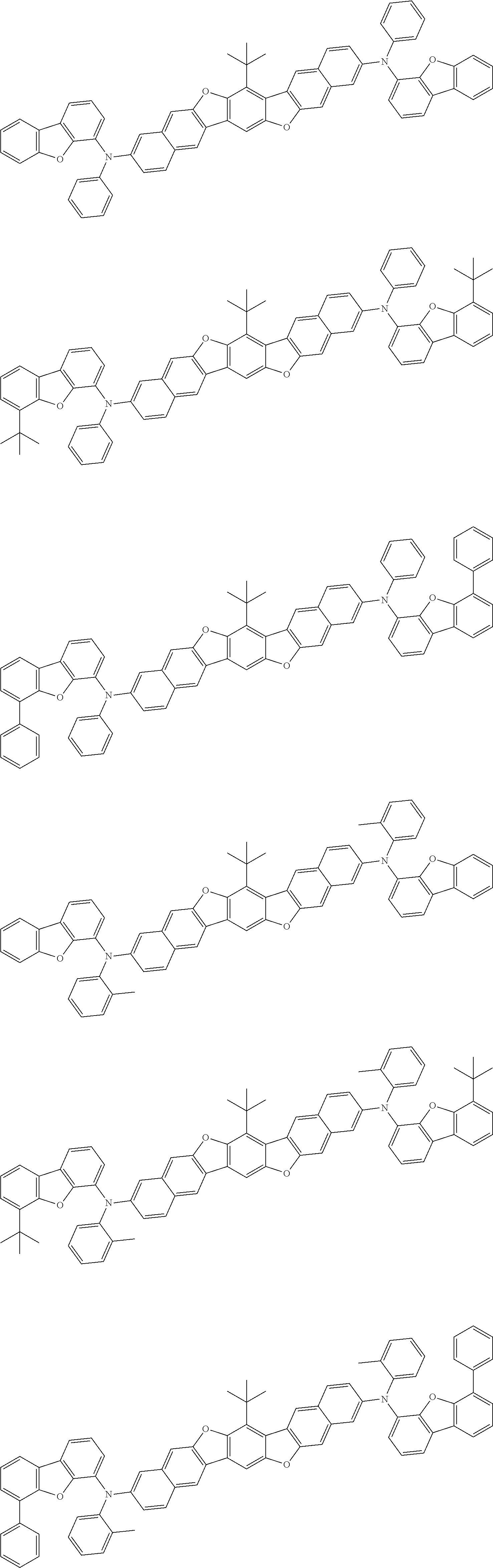

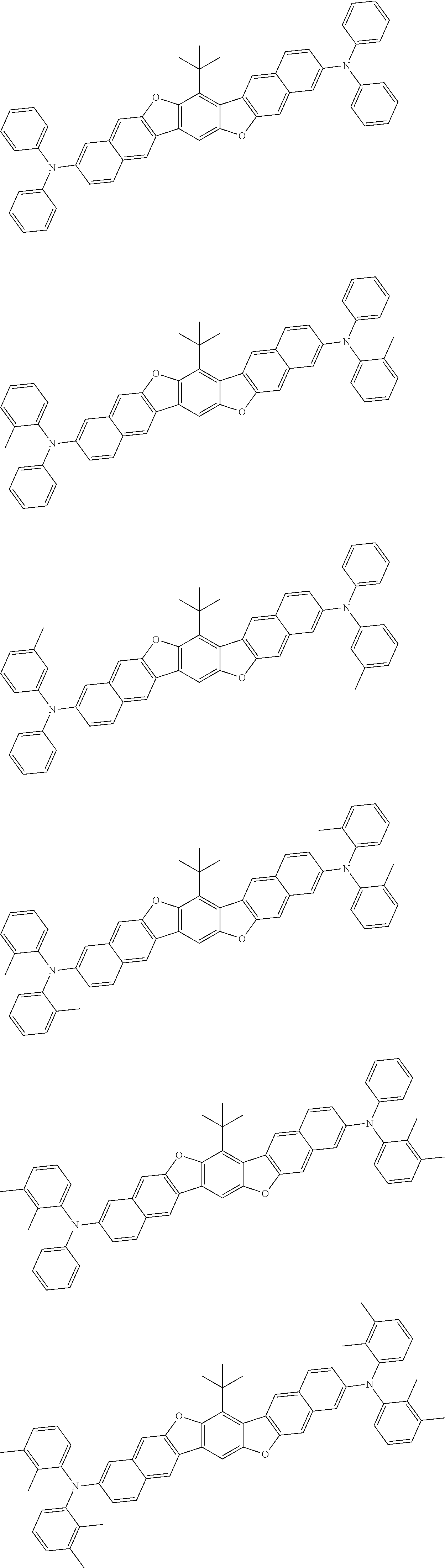

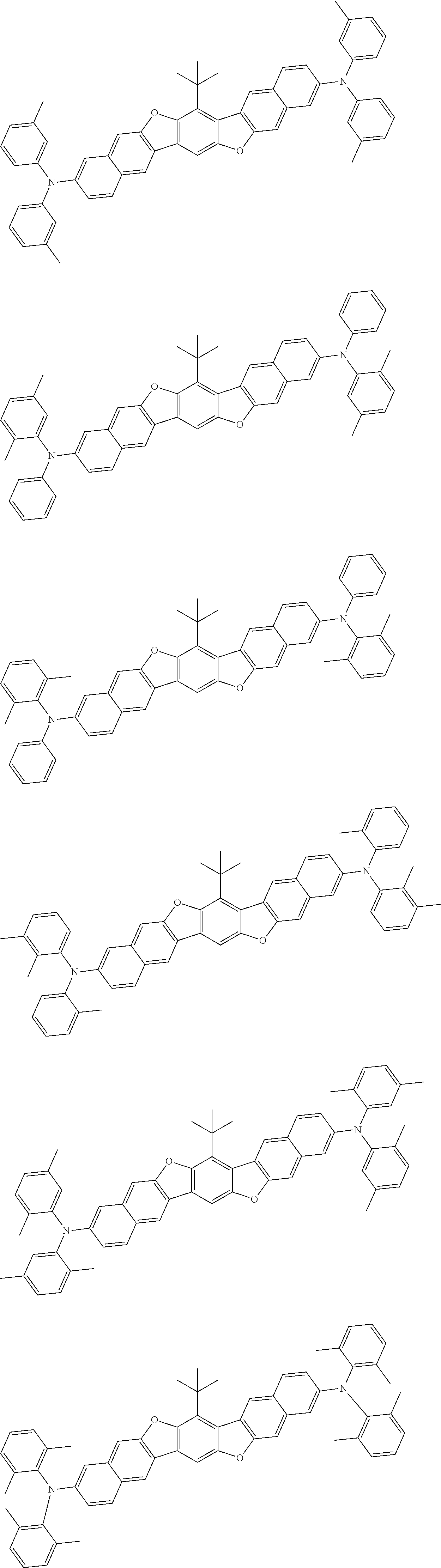

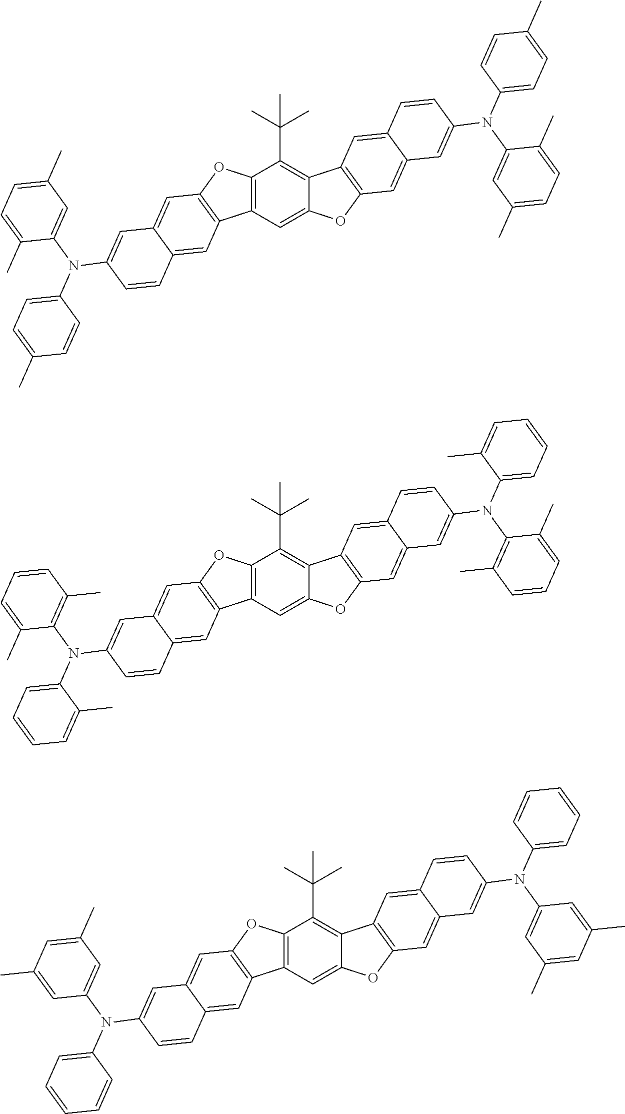

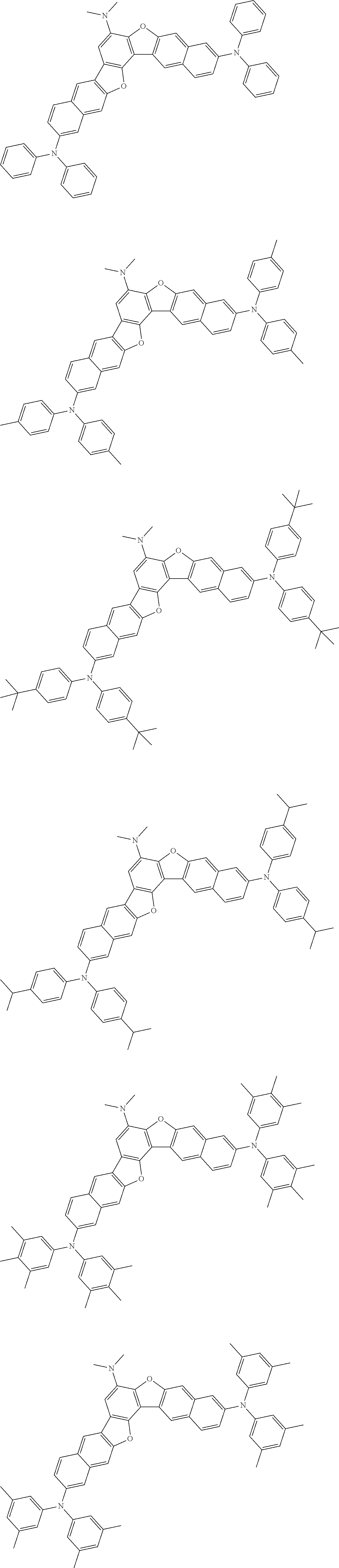

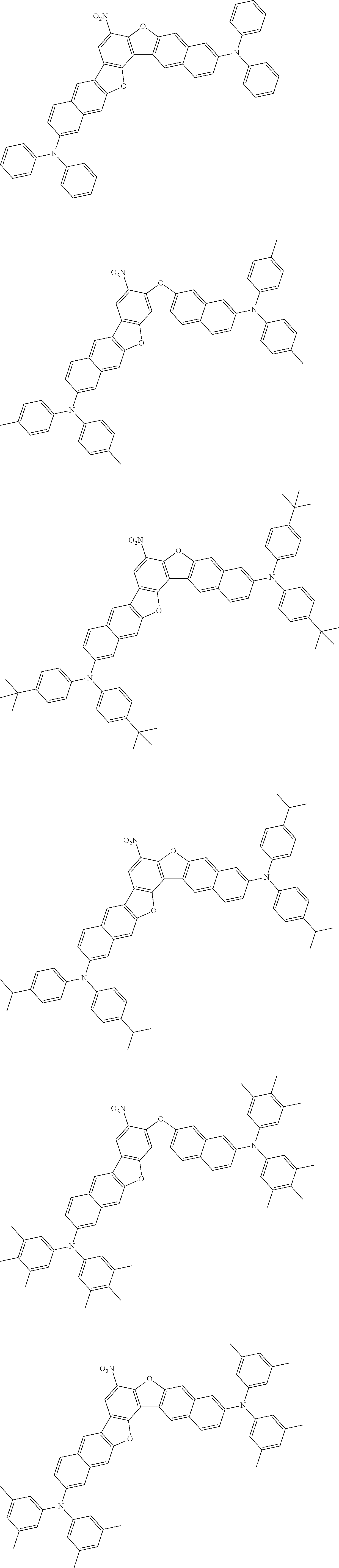

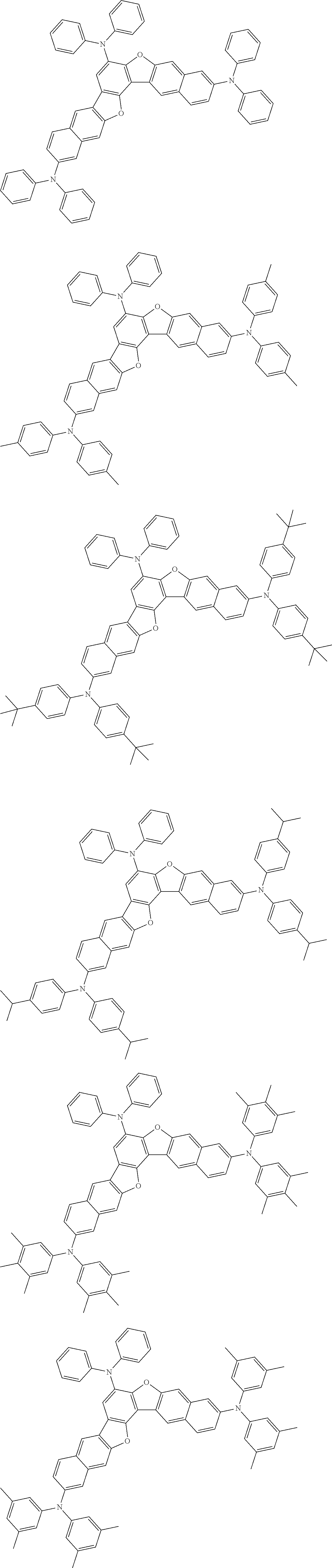

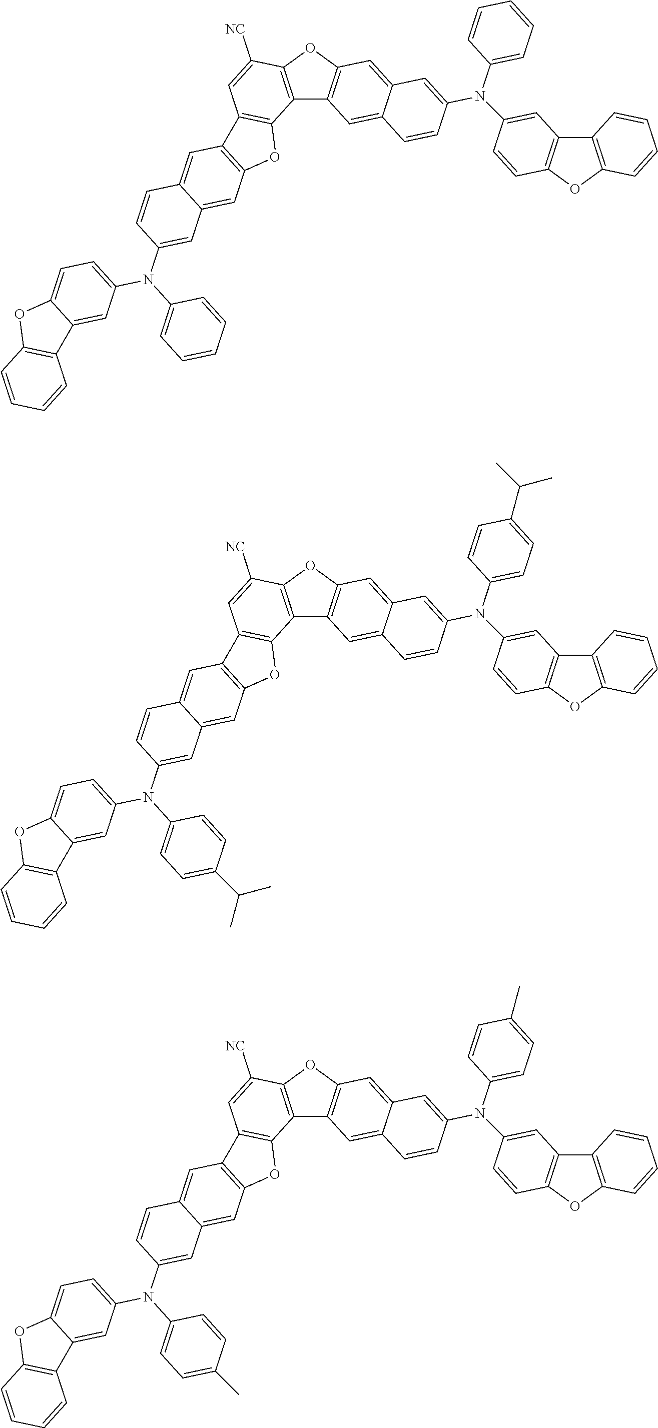

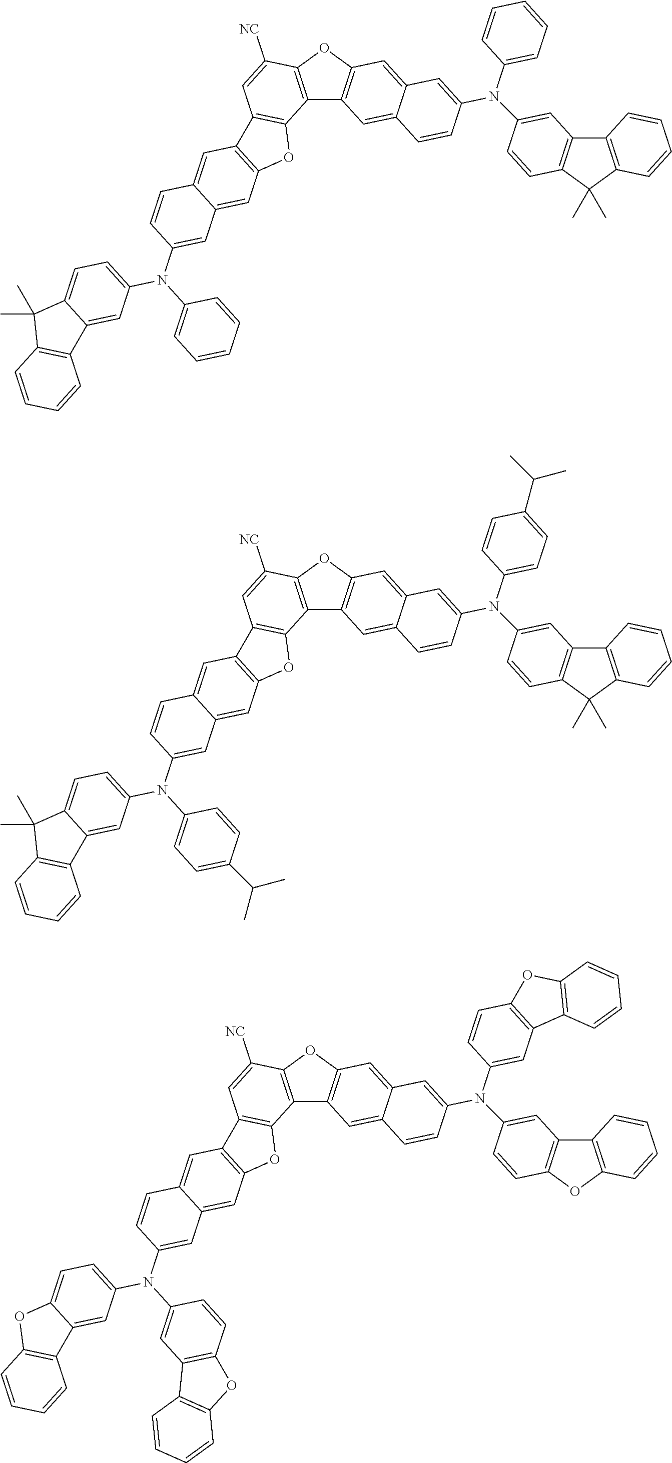

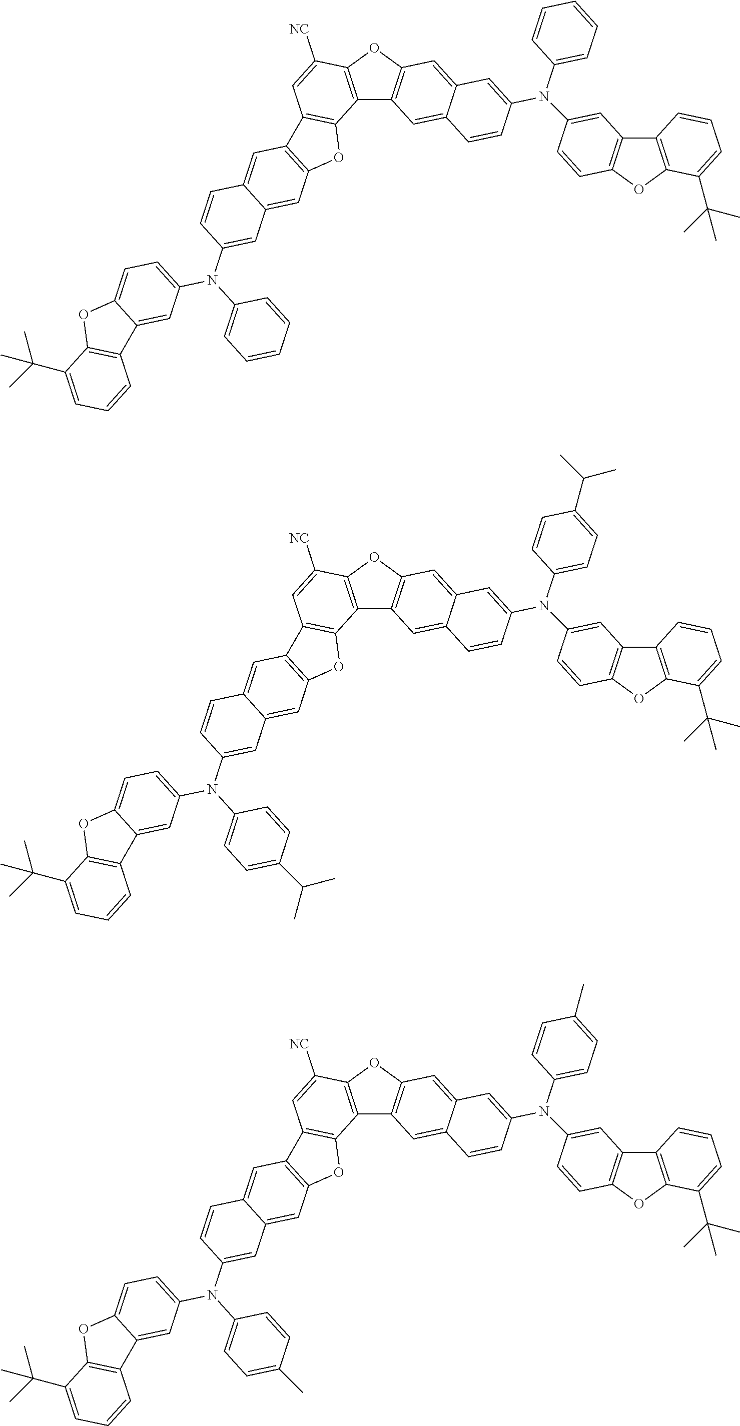

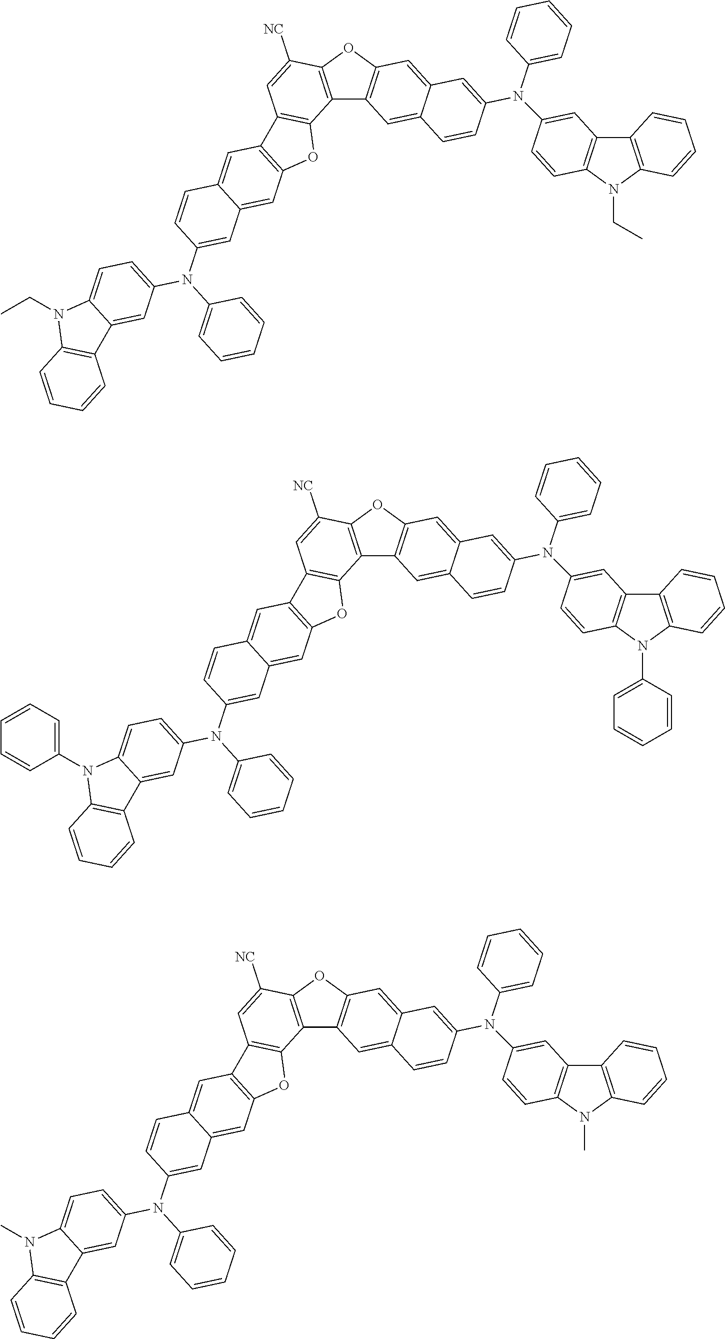

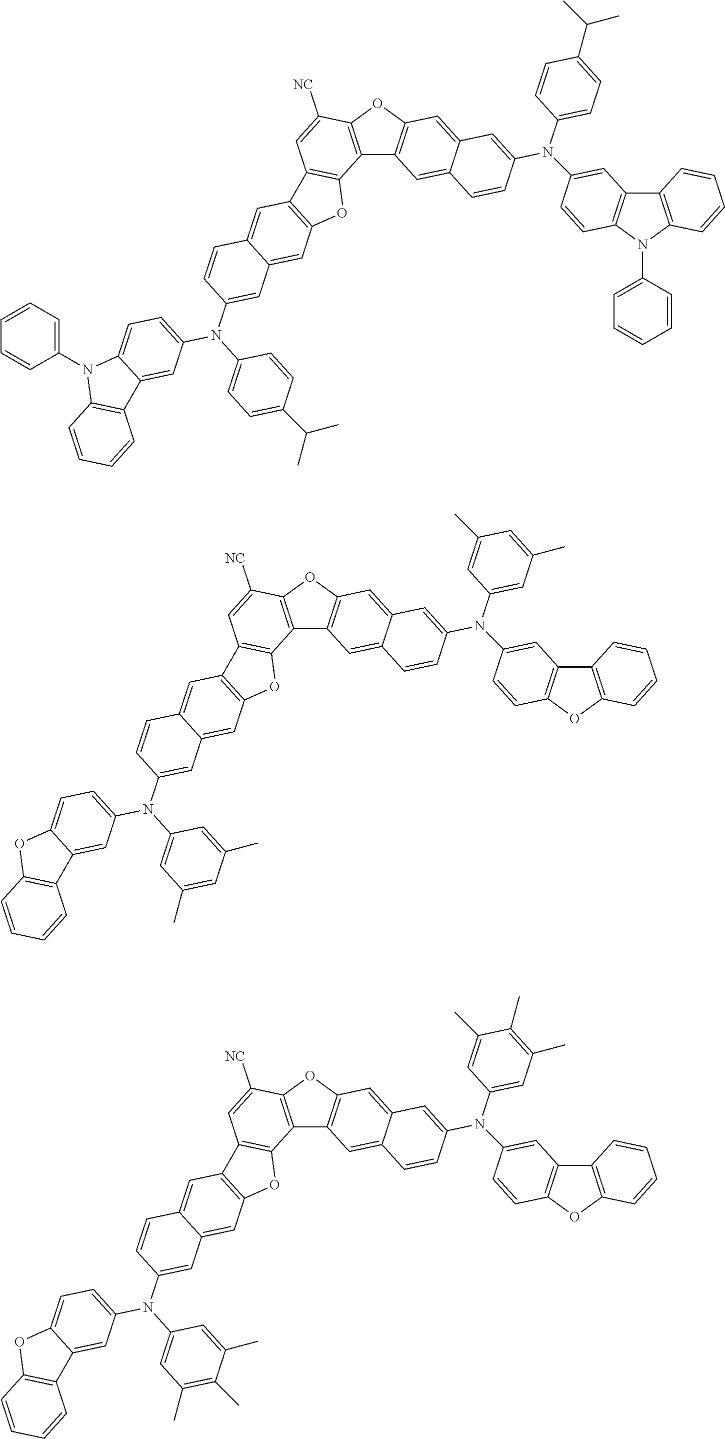

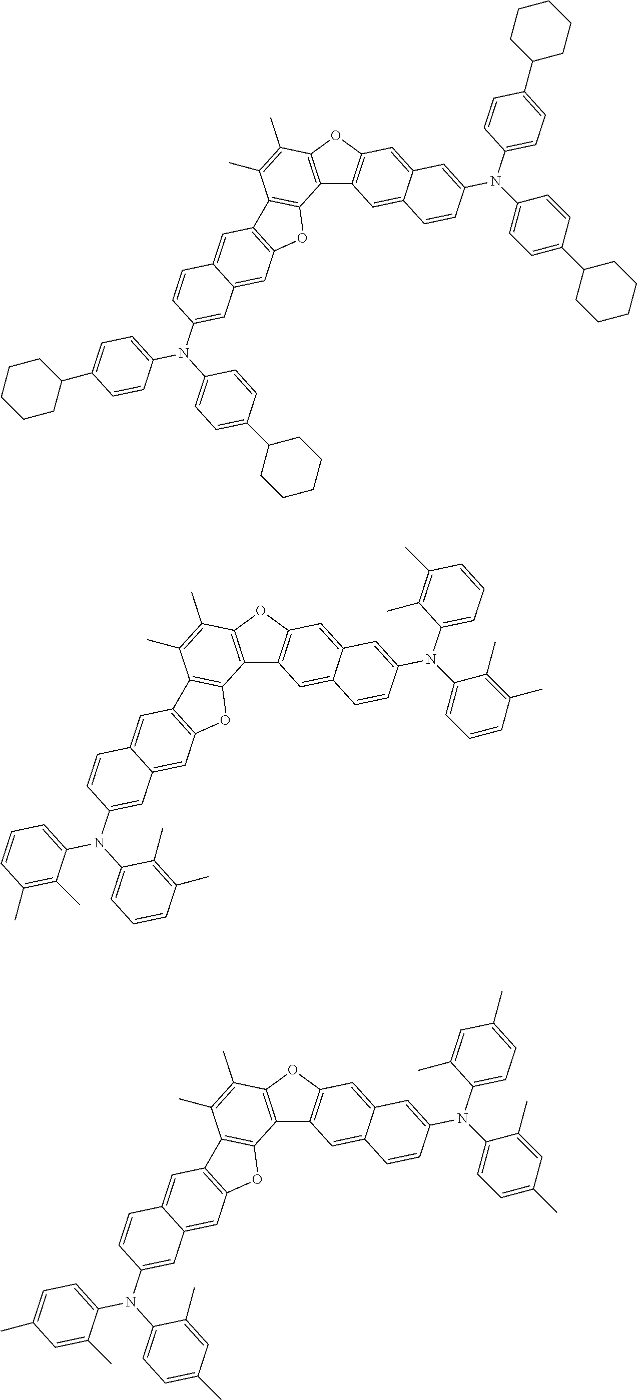

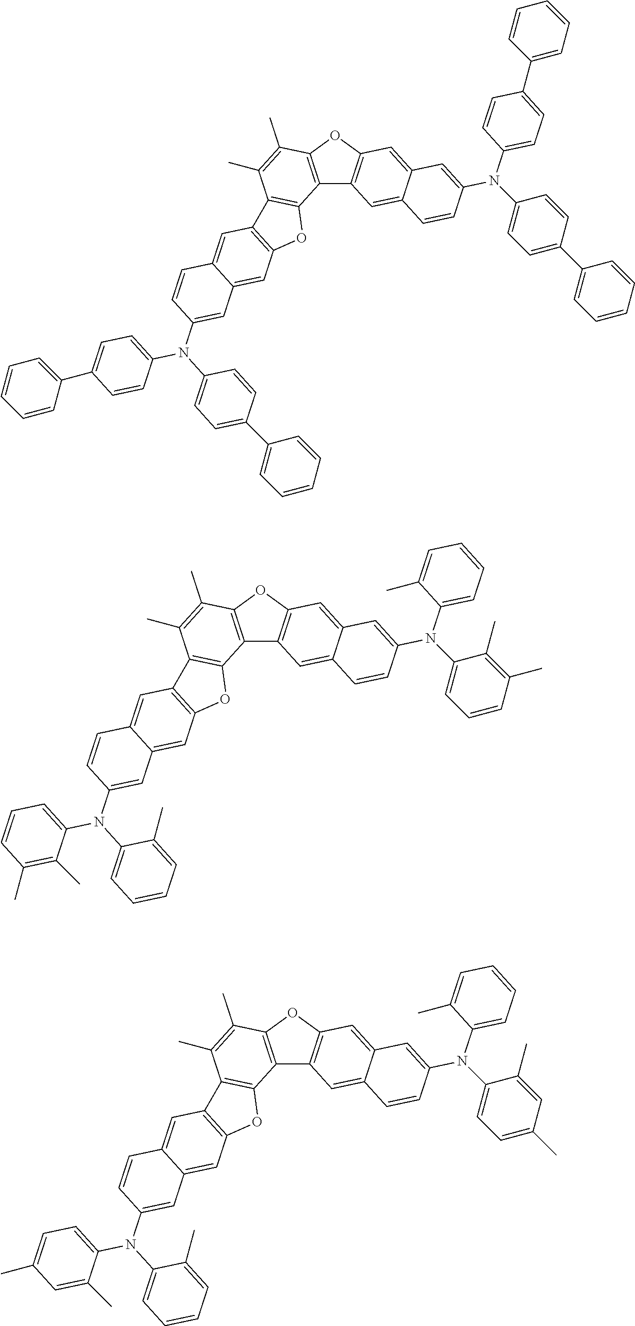

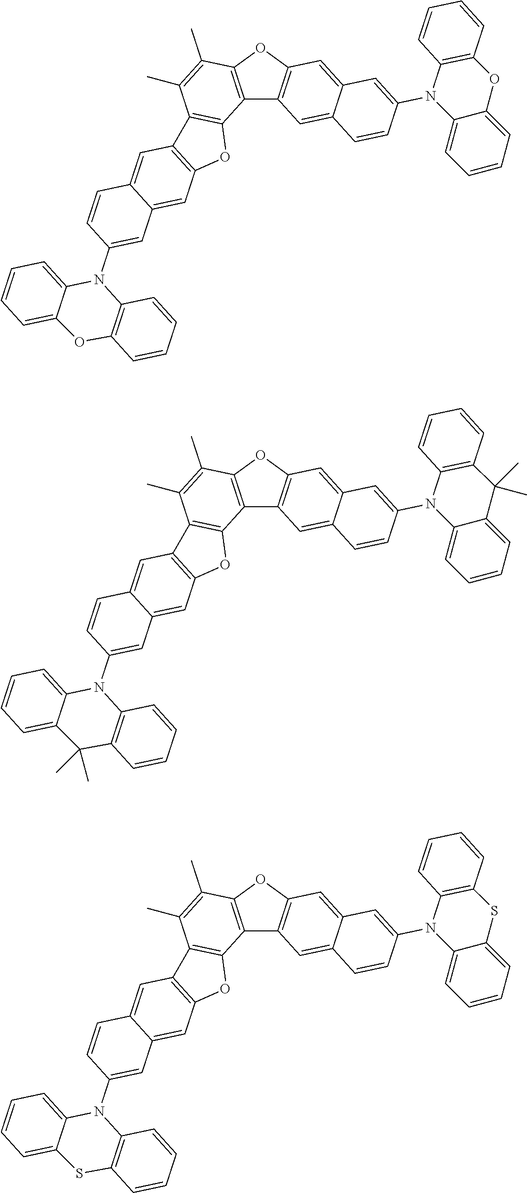

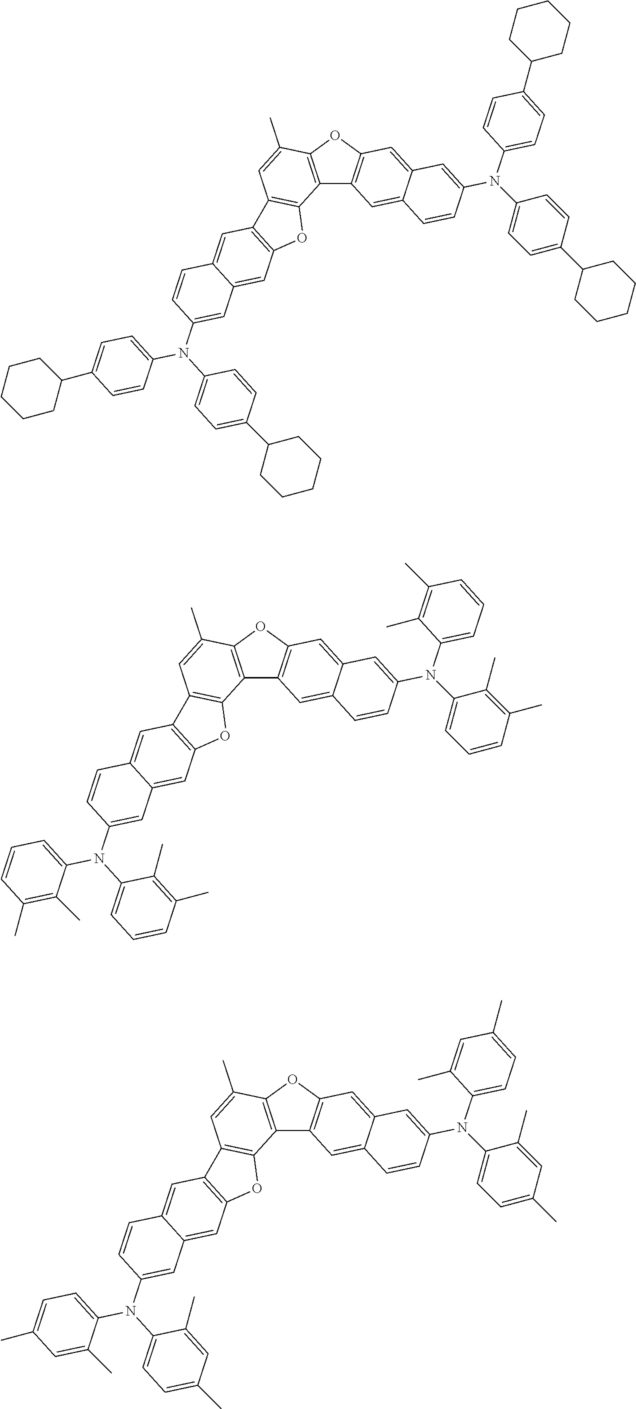

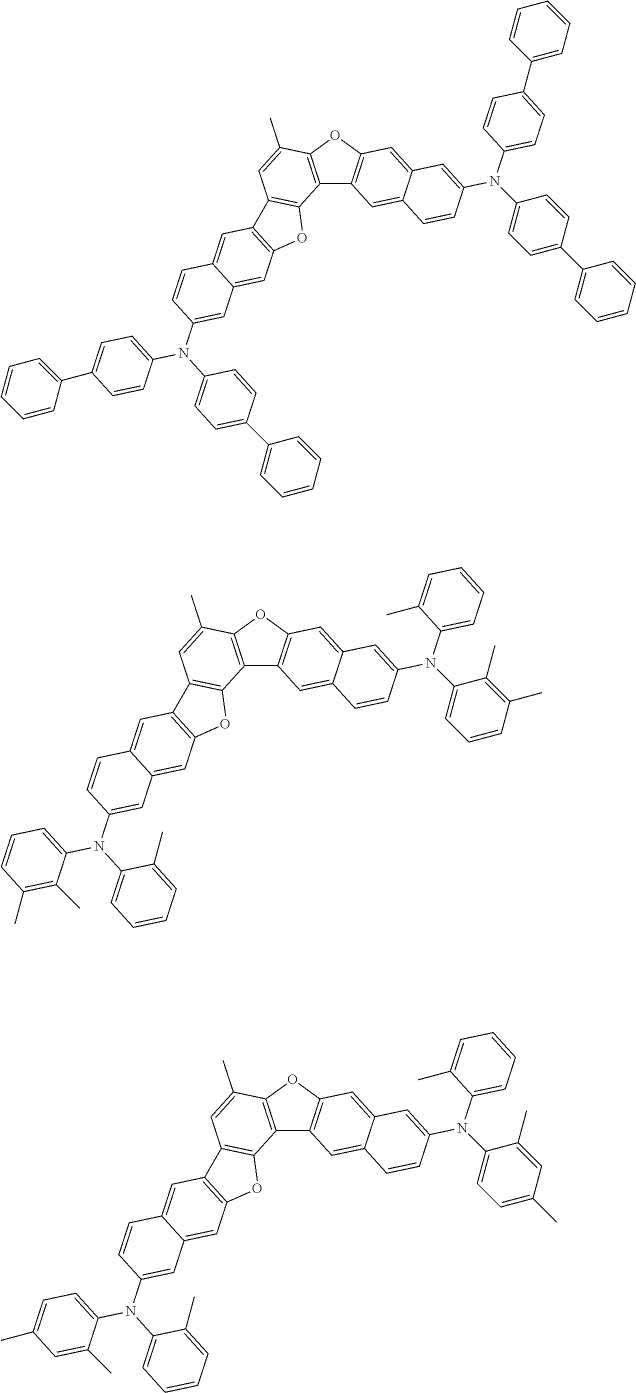

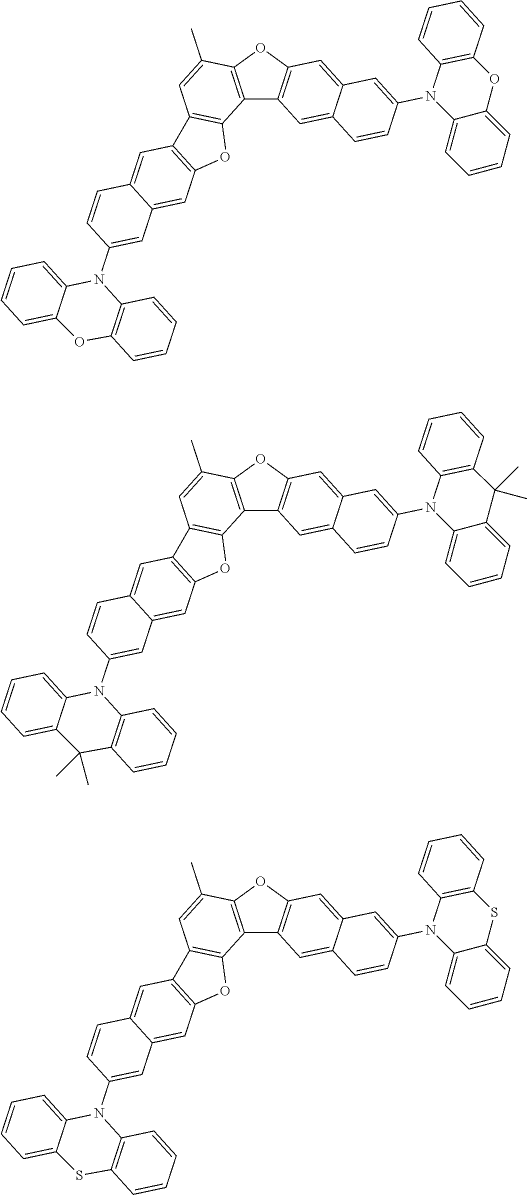

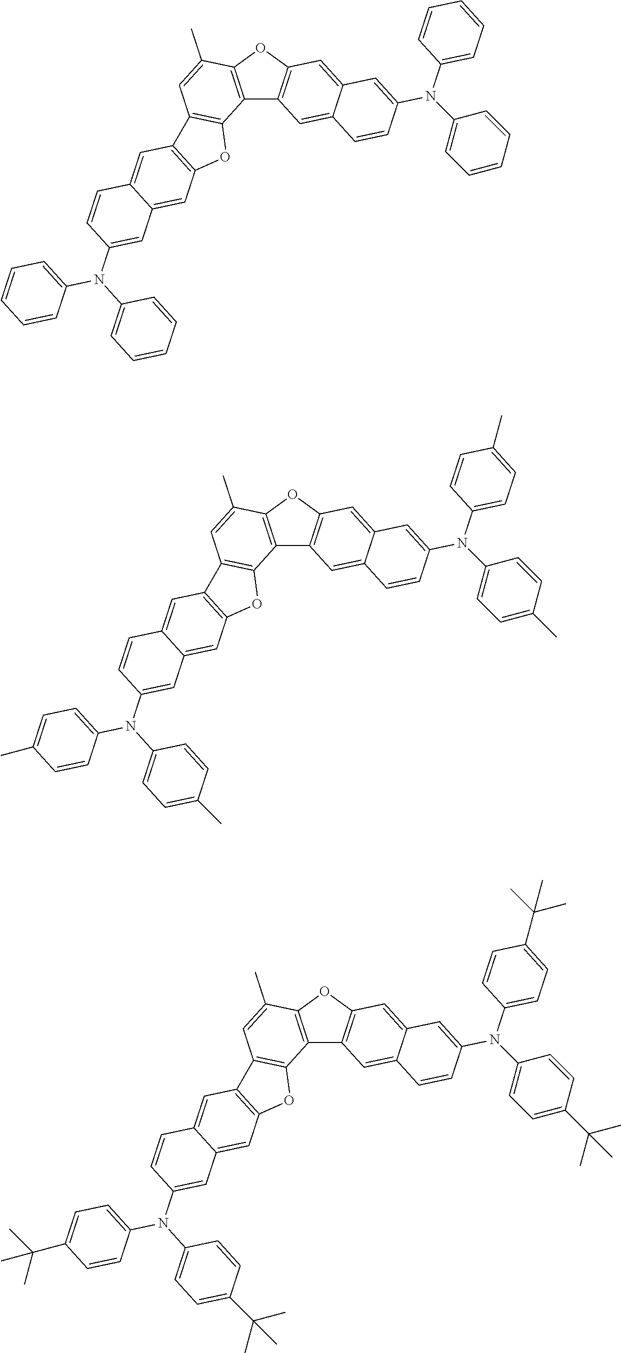

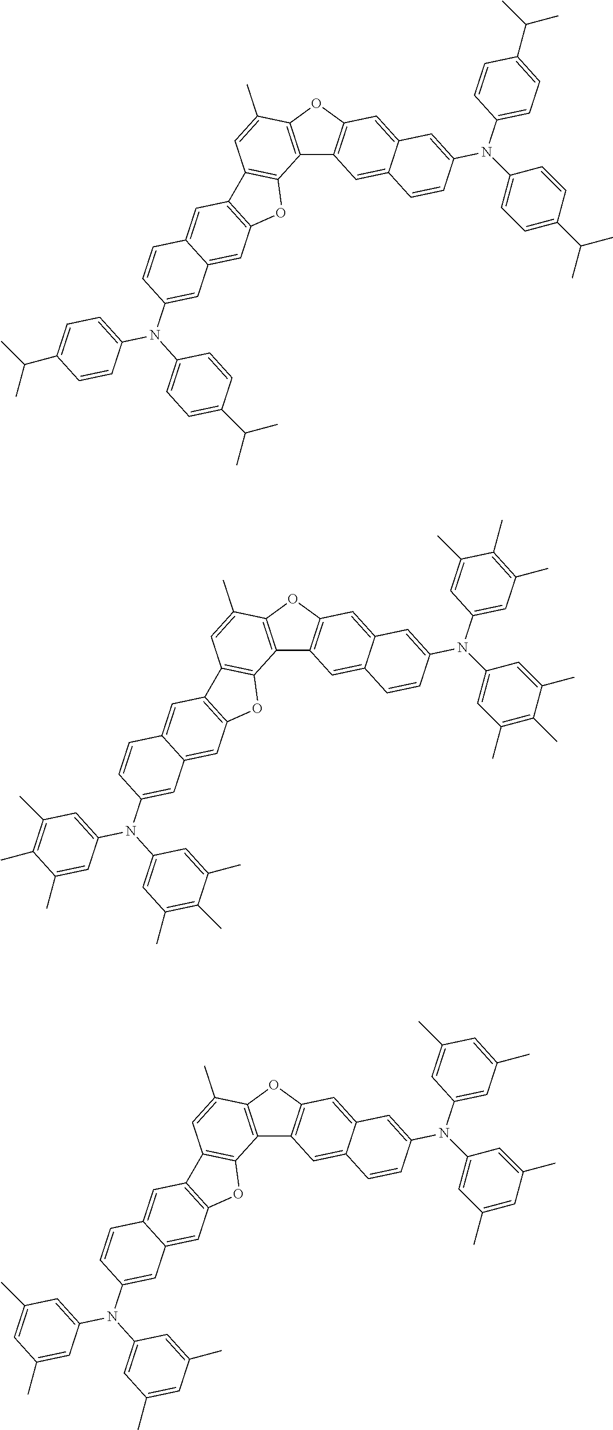

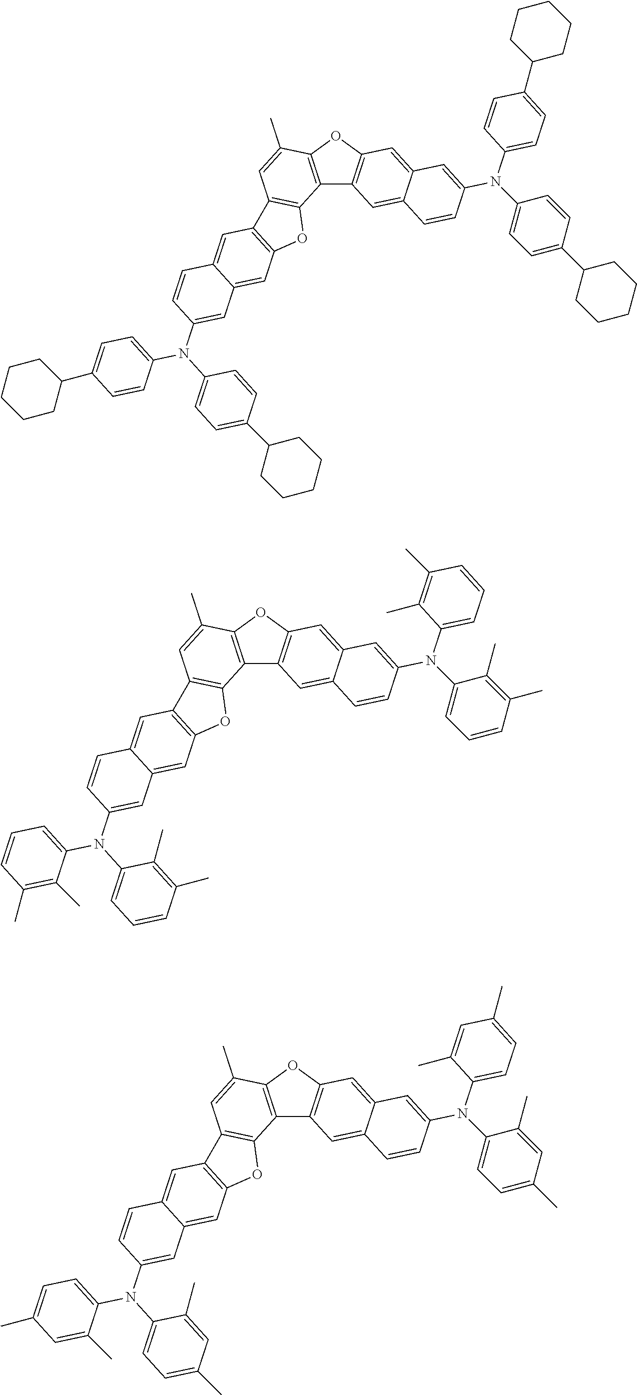

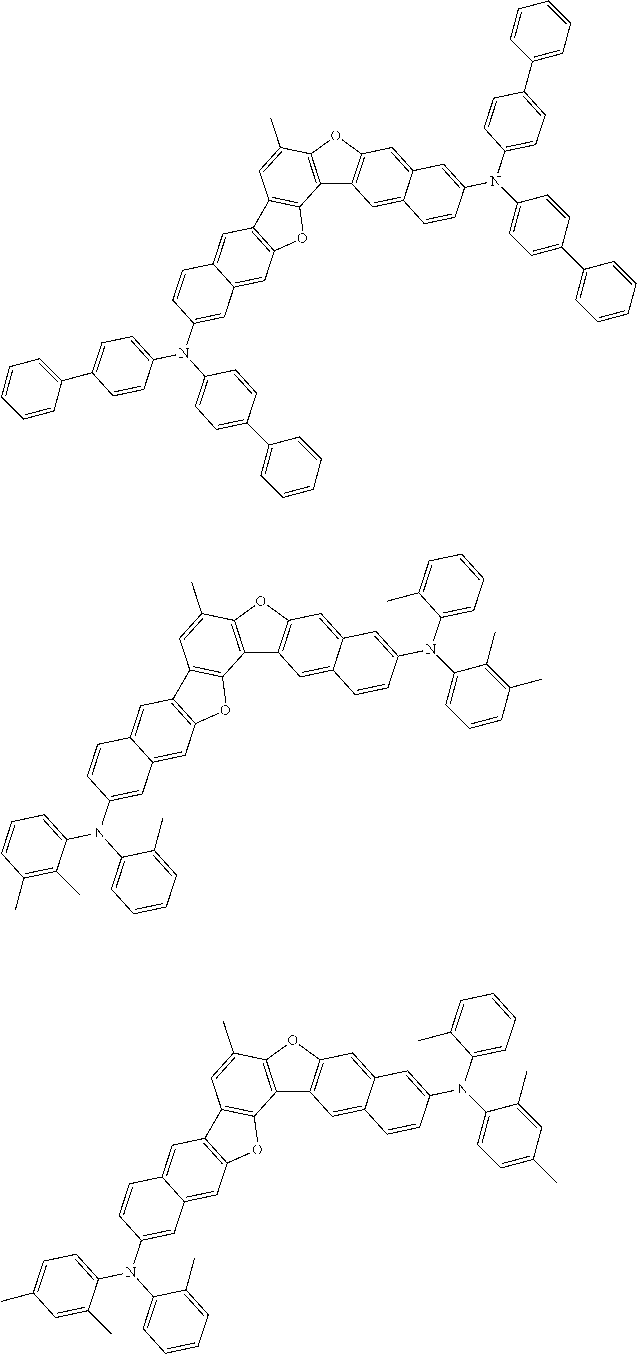

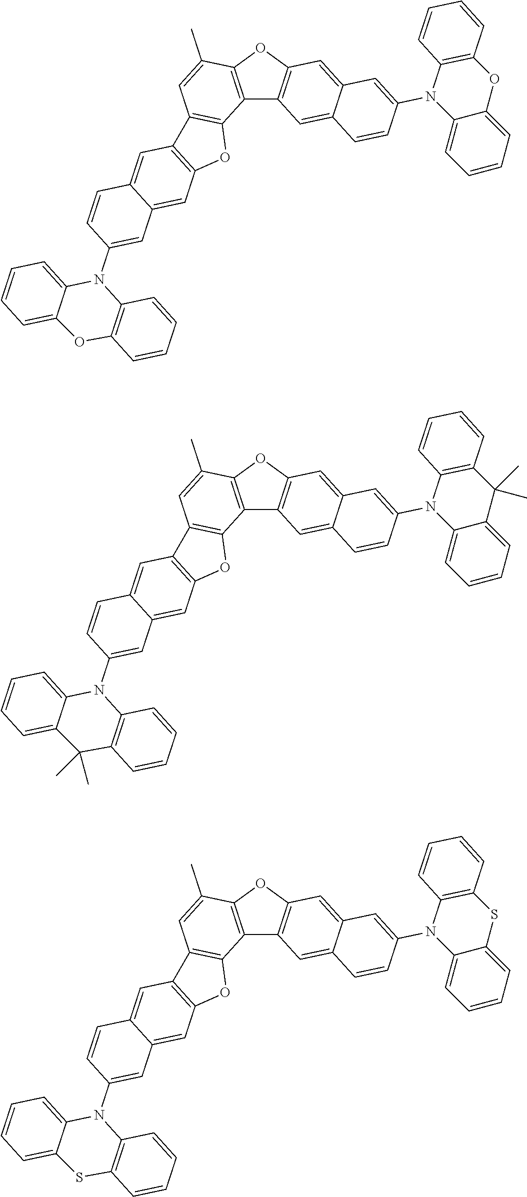

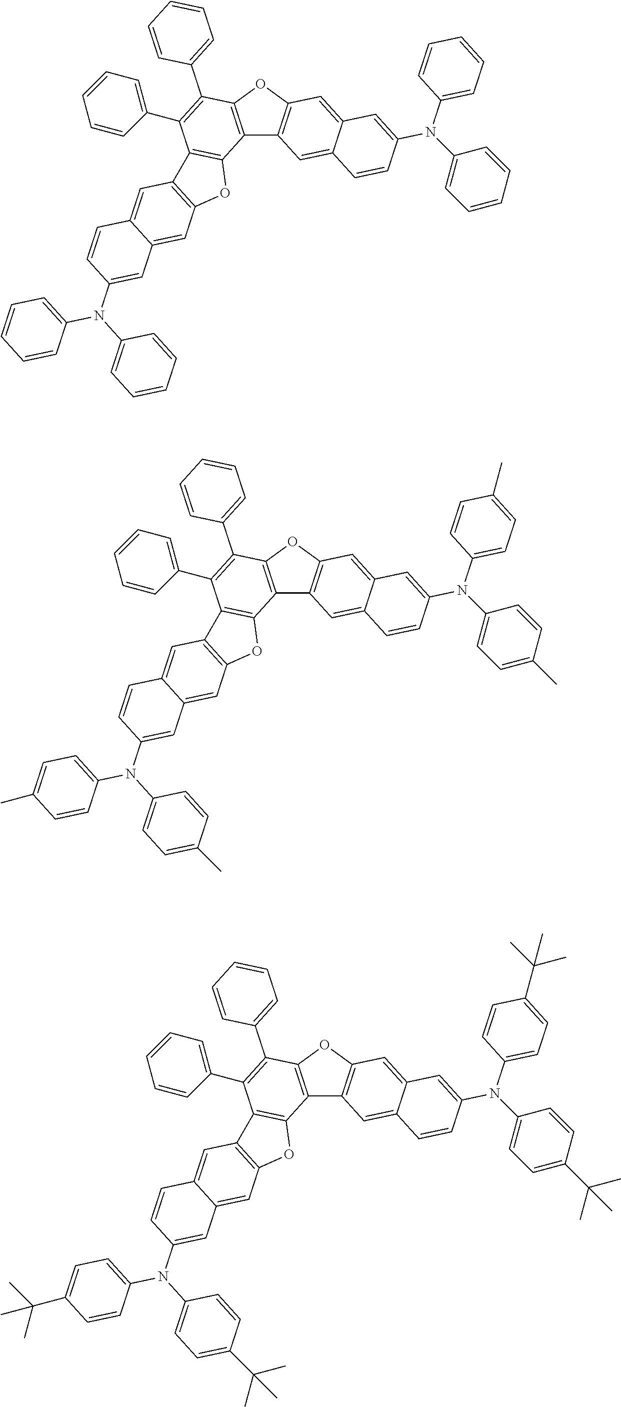

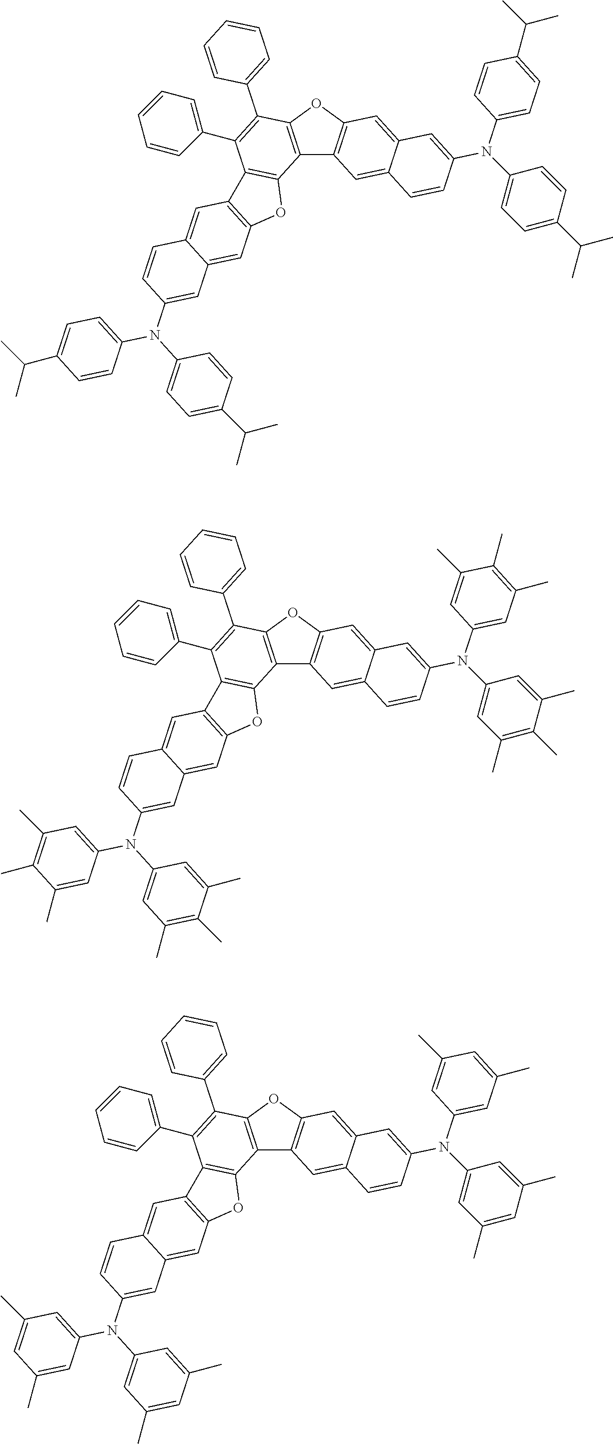

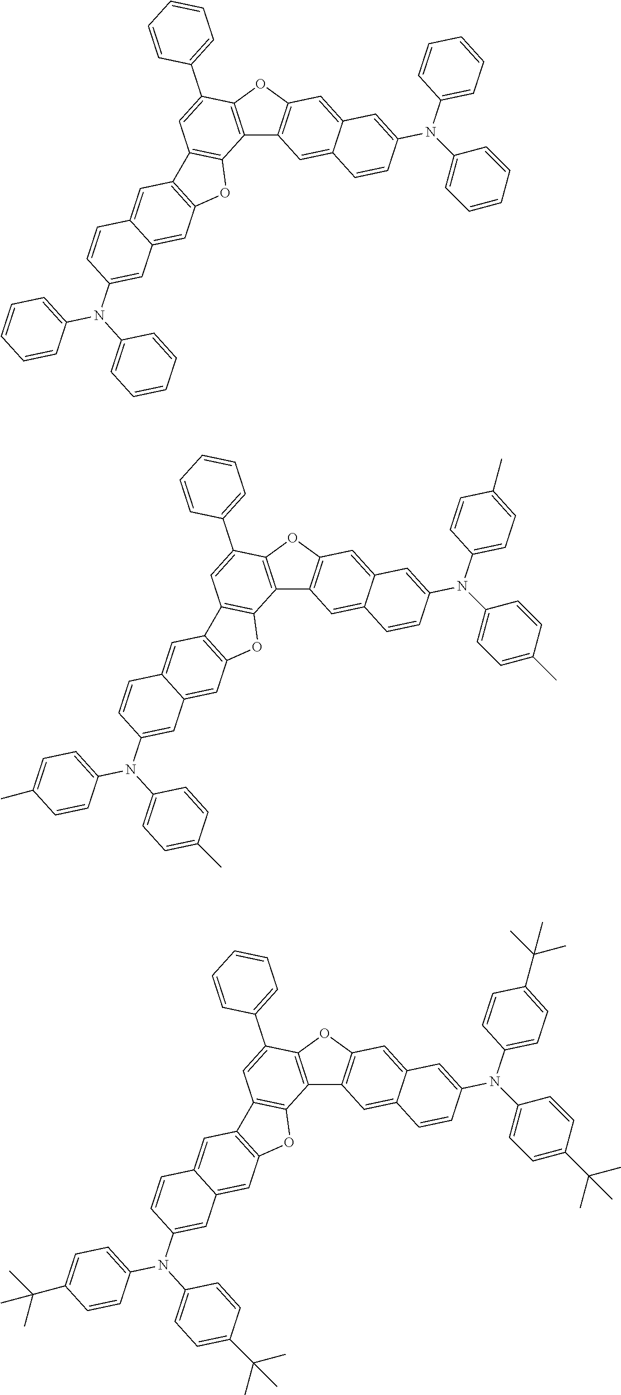

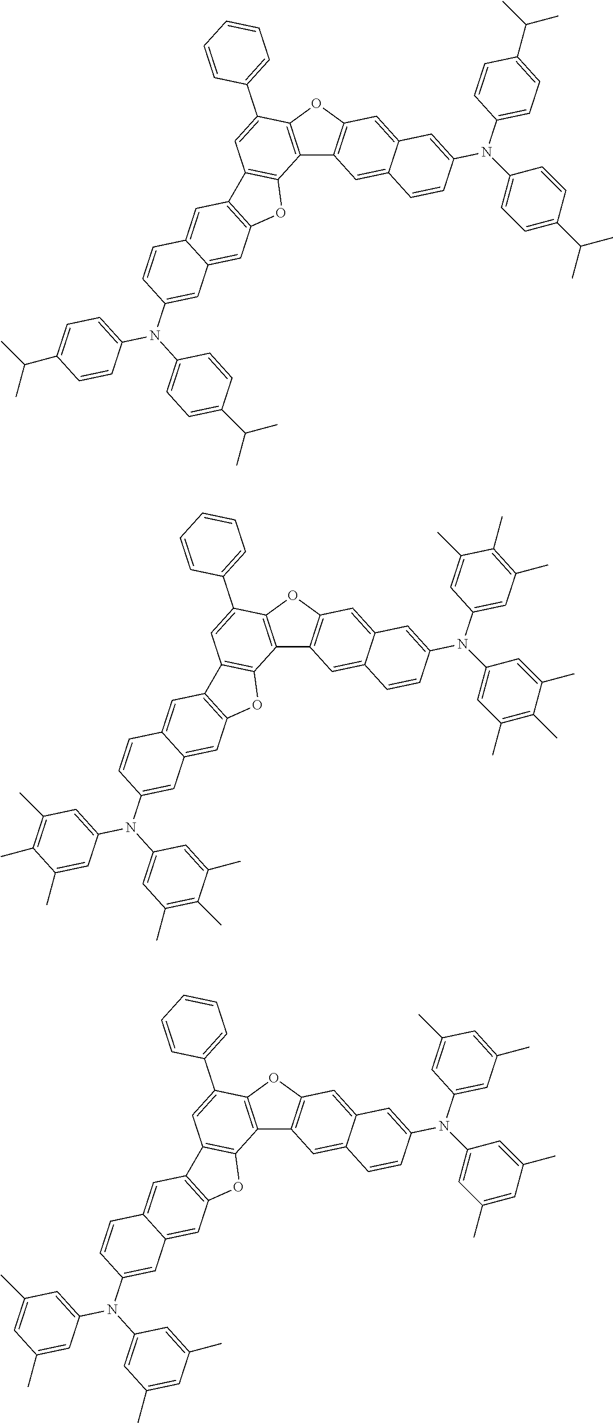

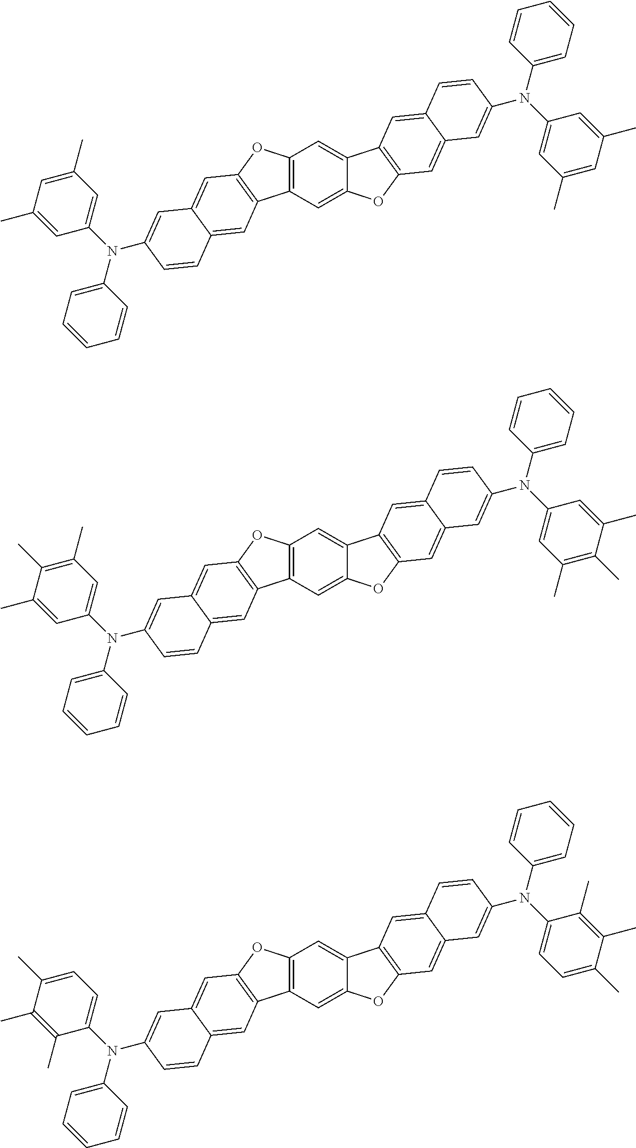

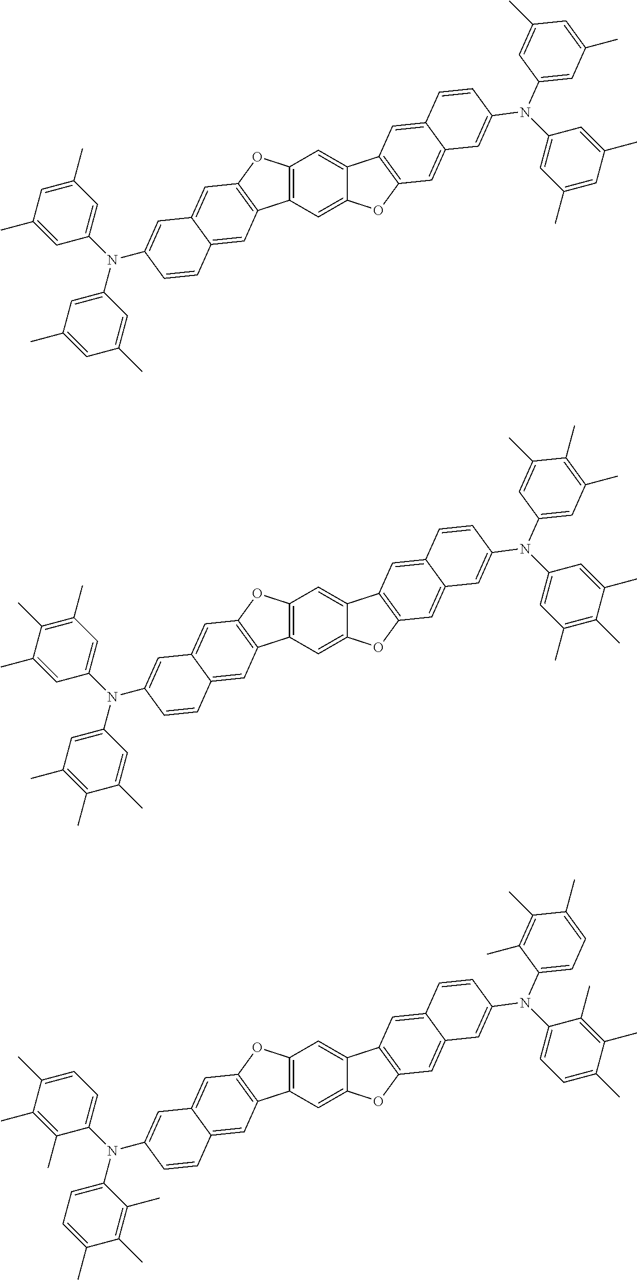

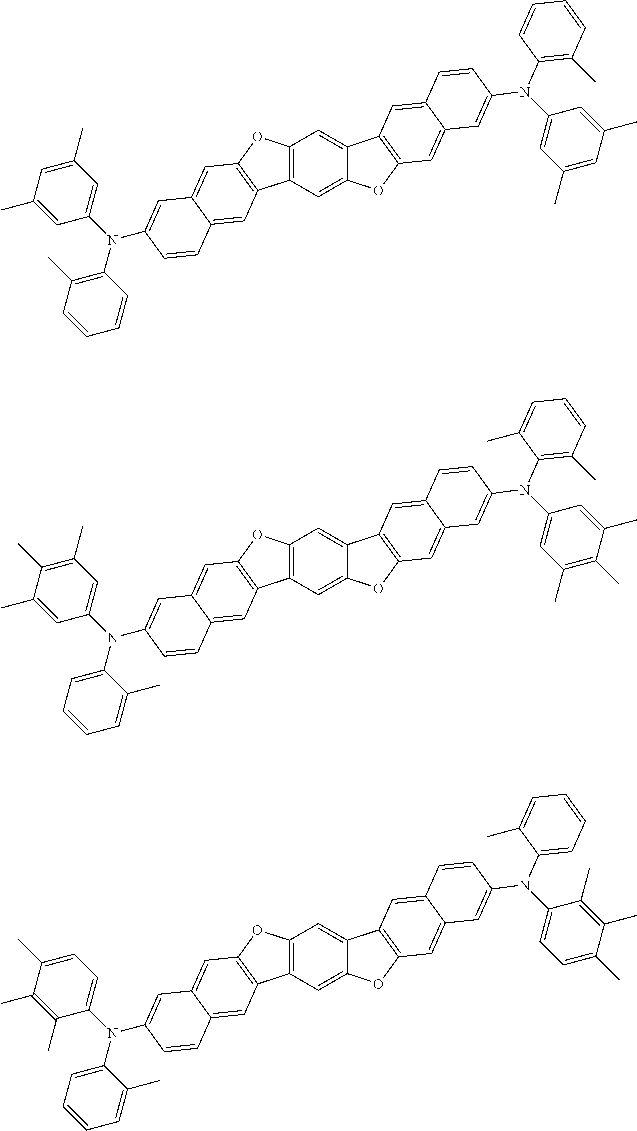

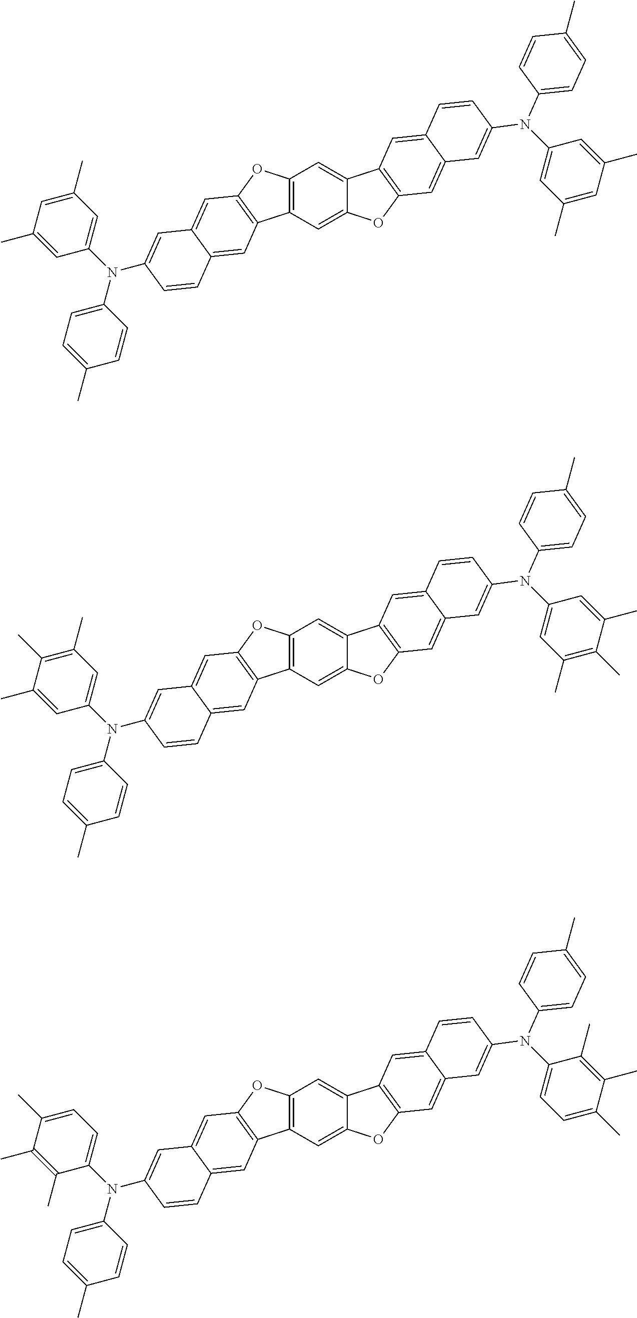

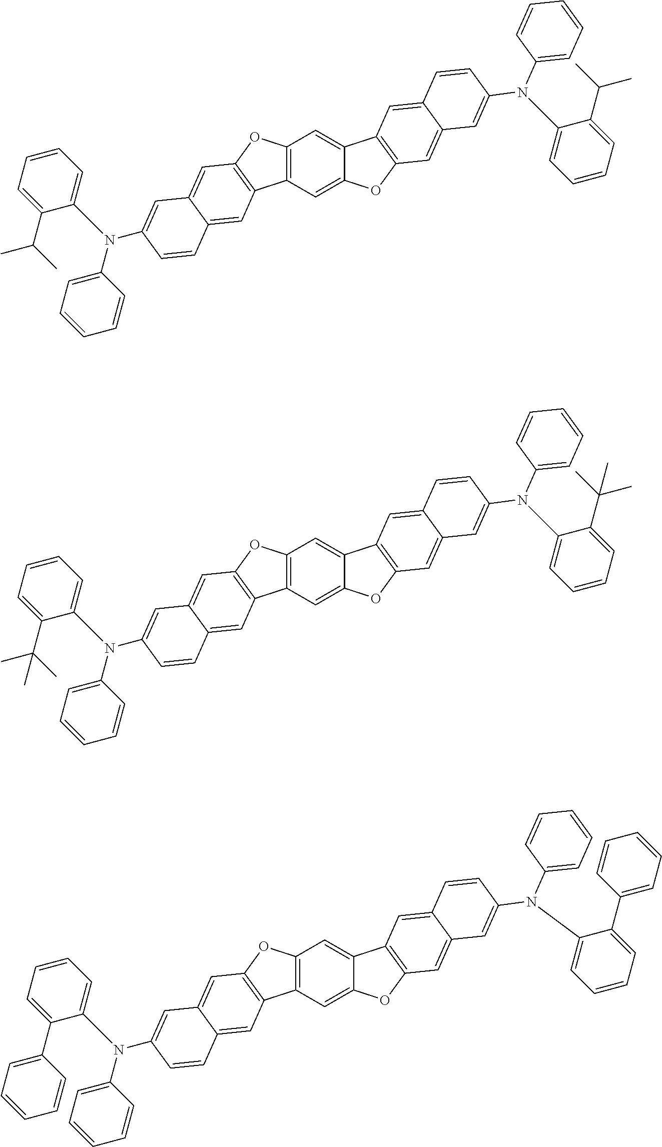

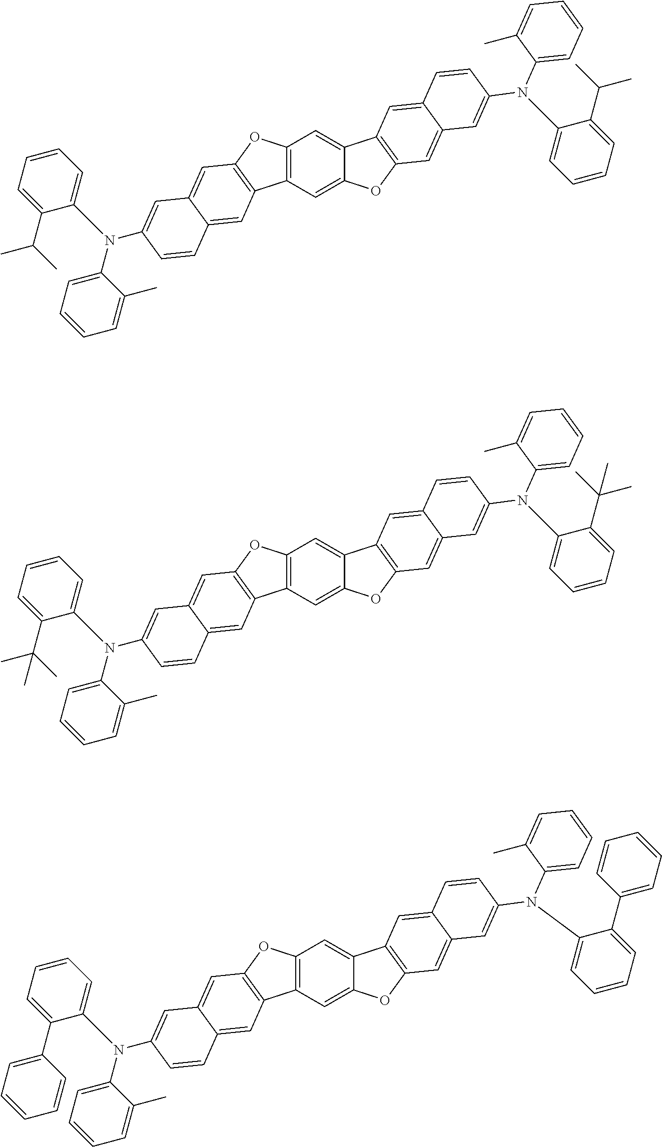

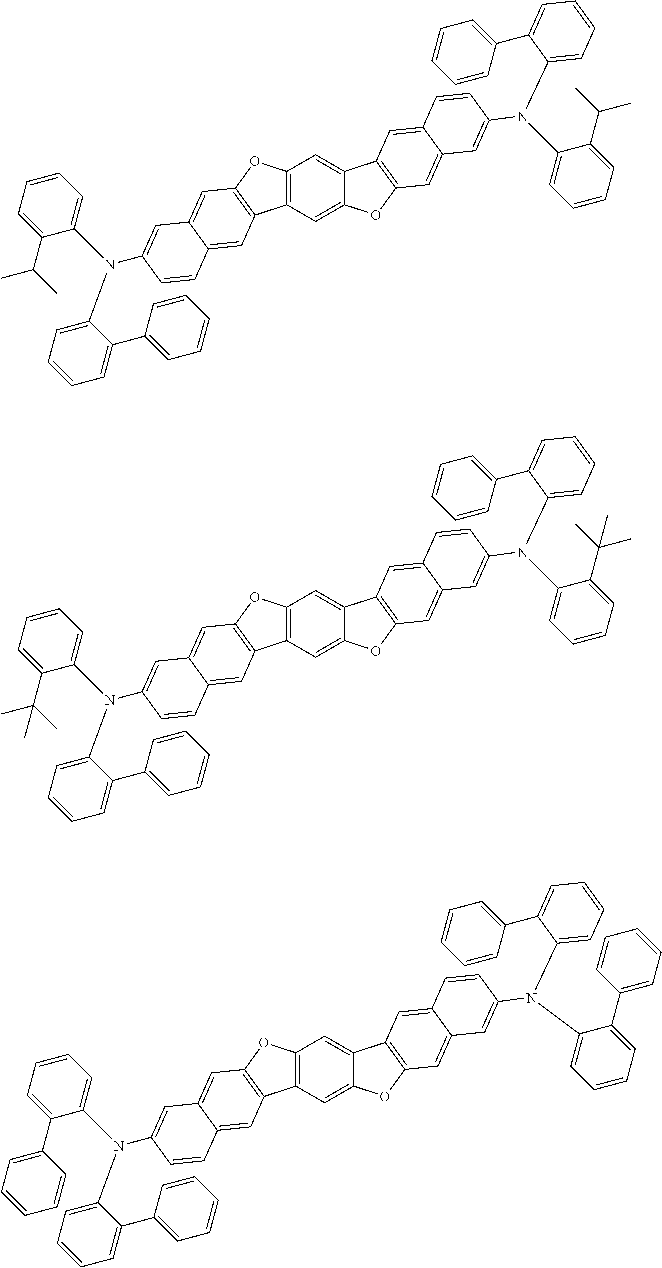

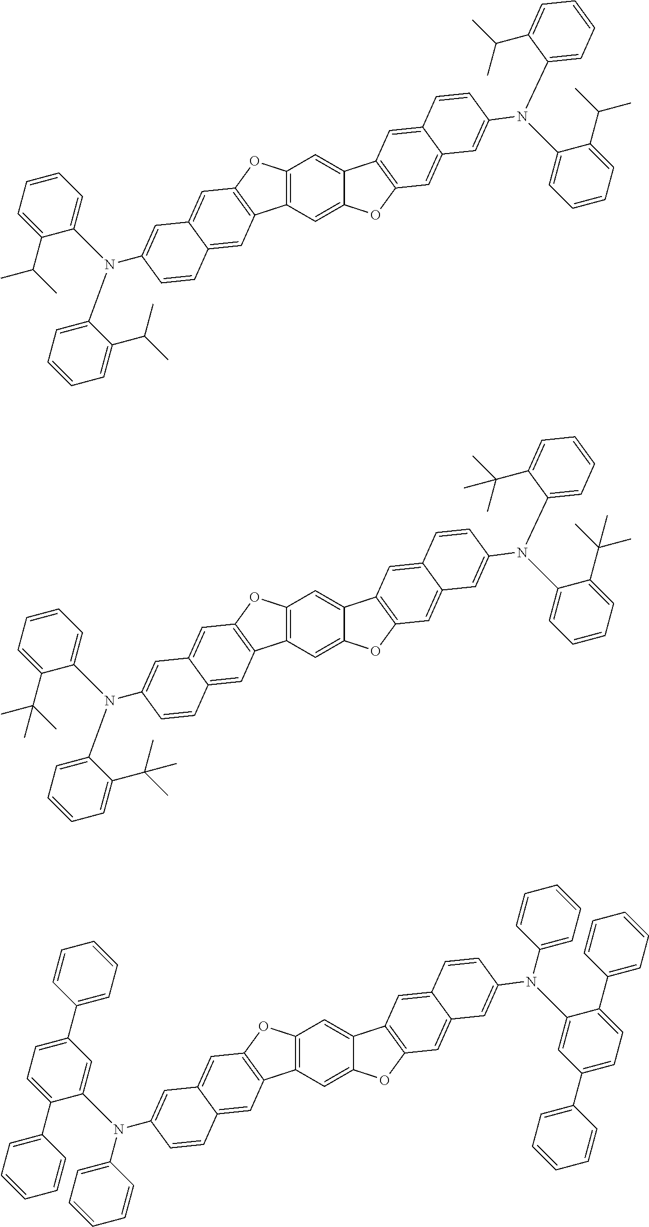

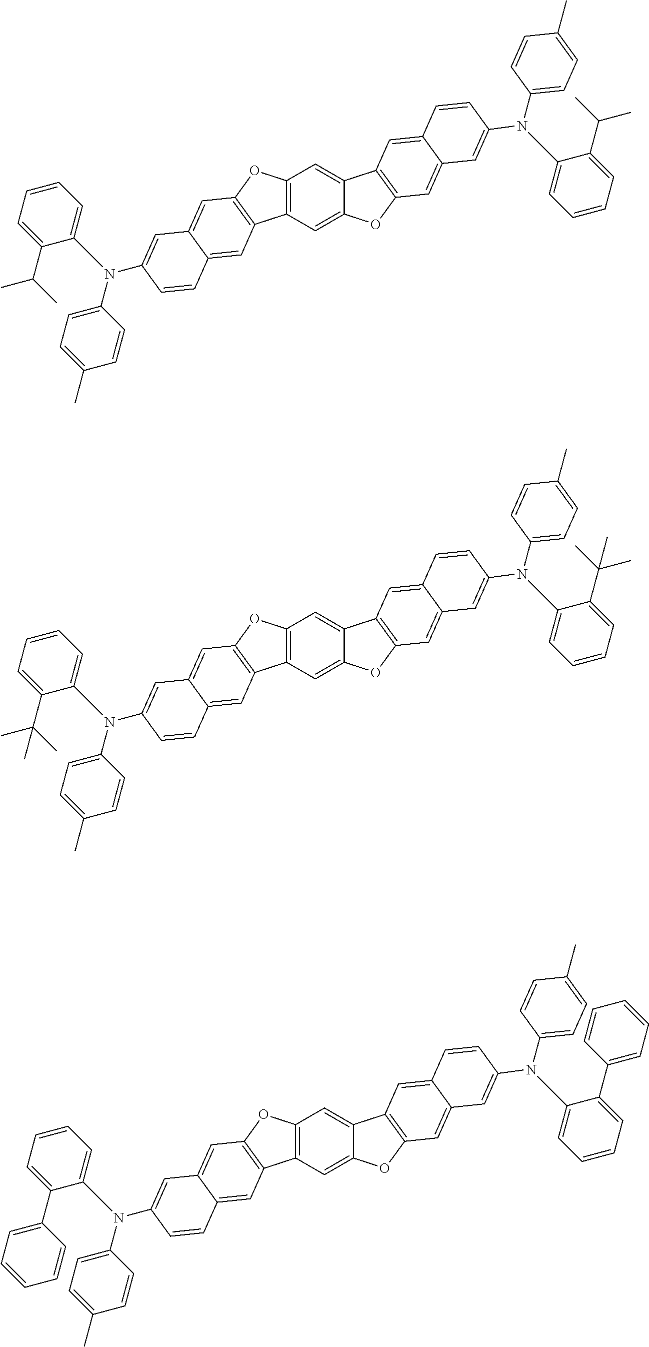

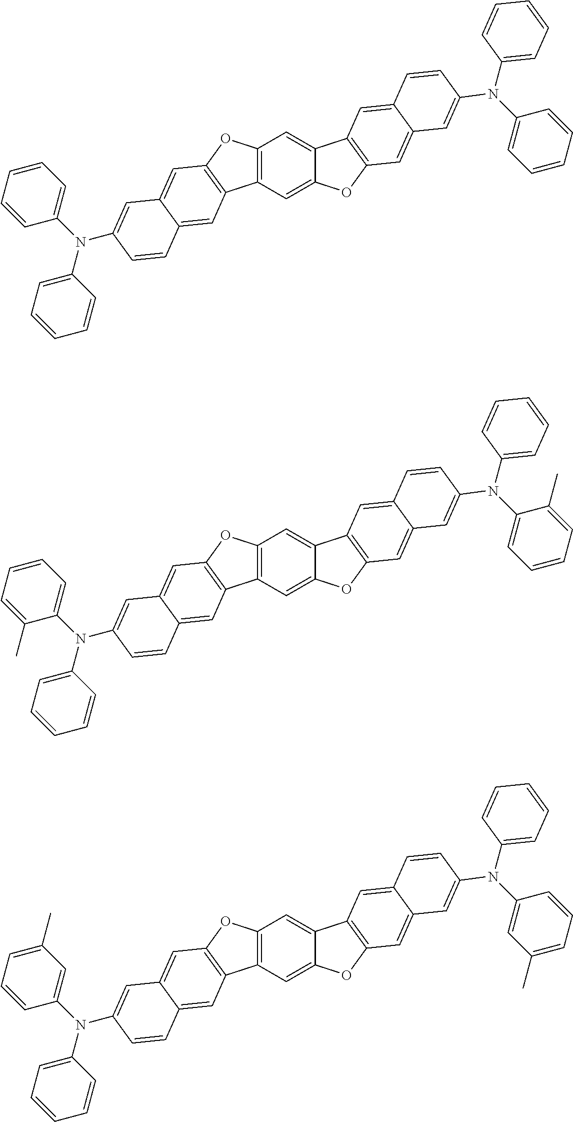 [SMILES: c1ccc(N(c2ccccc2)c2ccc3cc4c(cc3c2)oc2cc3c(cc24)oc2cc4cc(N(c5ccccc5)c5ccccc5)ccc4cc23)cc1 ; Cc1ccccc1N(c1ccccc1)c1ccc2cc3c(cc2c1)oc1cc2c(cc13)oc1cc3cc(N(c4ccccc4)c4ccccc4C)ccc3cc12 ; Cc1cccc(N(c2ccccc2)c2ccc3cc4c(cc3c2)oc2cc3c(cc24)oc2cc4cc(N(c5ccccc5)c5cccc(C)c5)ccc4cc23)c1]

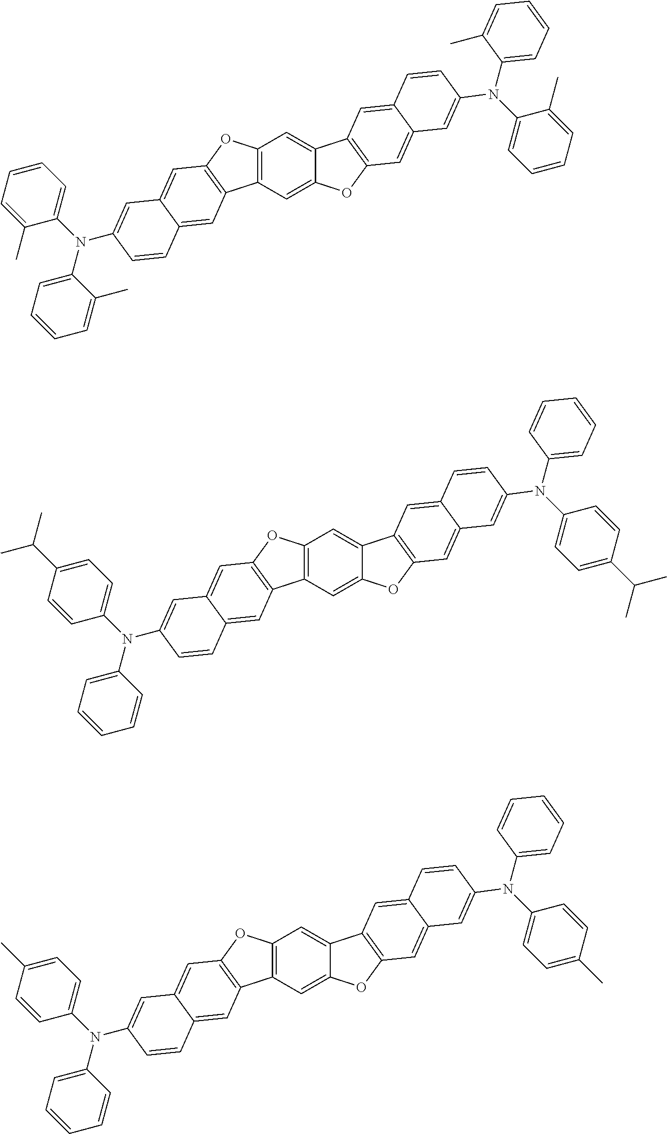

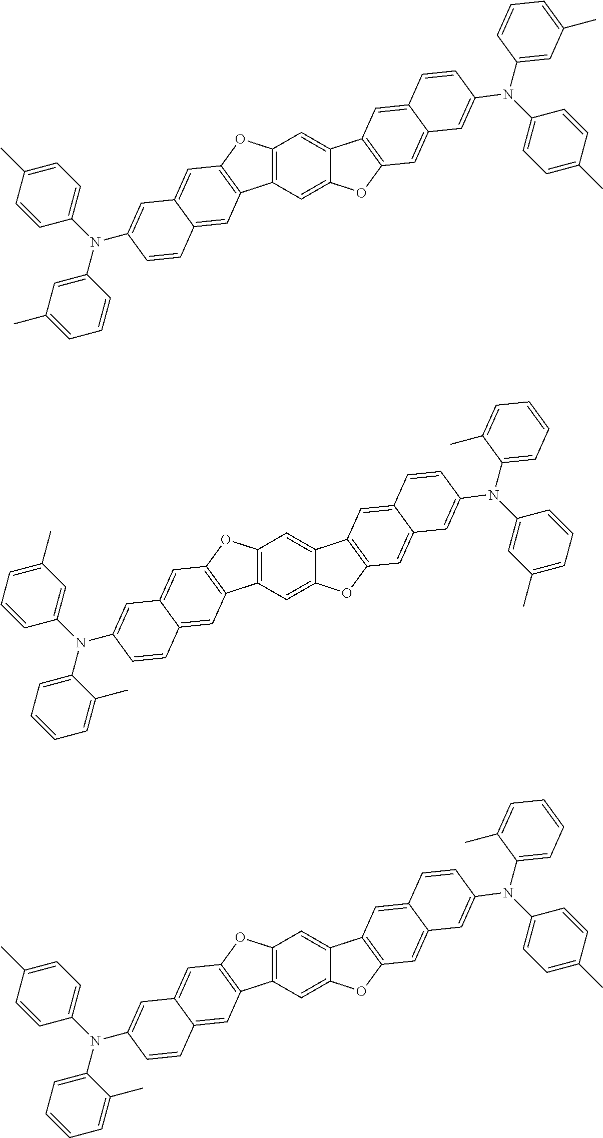

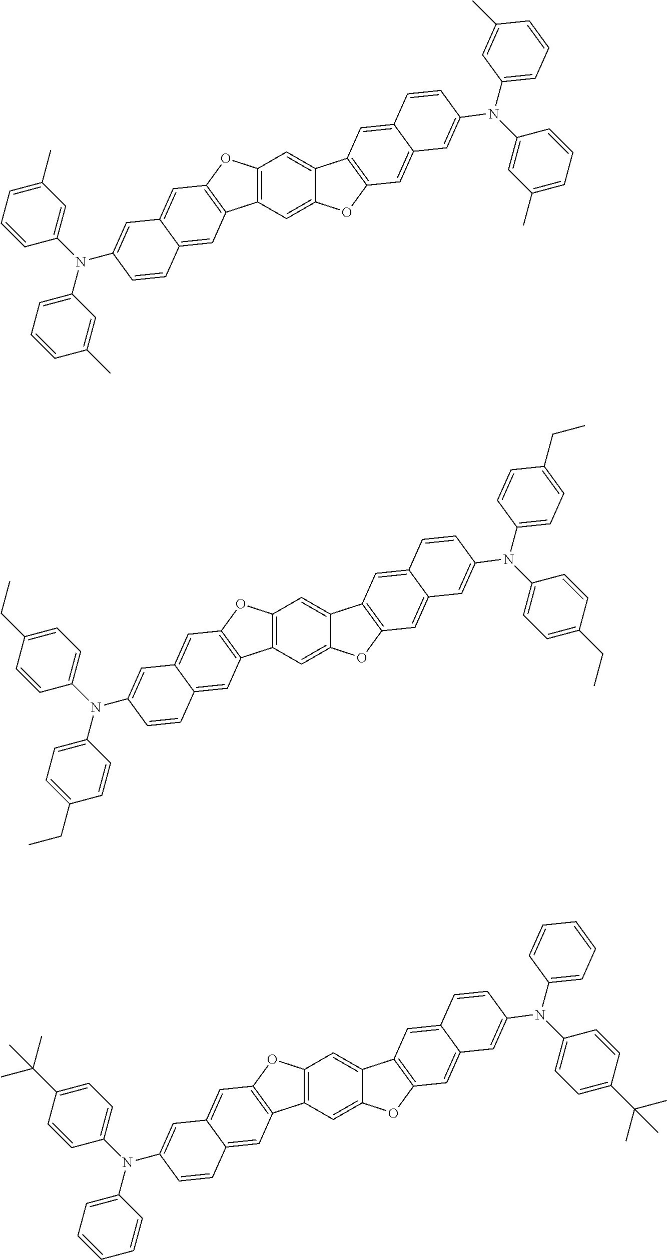 [SMILES: Cc1cccc(N(c2cccc(C)c2)c2ccc3cc4c(cc3c2)oc2cc3c(cc24)oc2cc4cc(N(c5cccc(C)c5)c5cccc(C)c5)ccc4cc23)c1 ; CCc1ccc(N(c2ccc(CC)cc2)c2ccc3cc4c(cc3c2)oc2cc3c(cc24)oc2cc4cc(N(c5ccc(CC)cc5)c5ccc(CC)cc5)ccc4cc23)cc1 ; CC(C)(C)c1ccc(N(c2ccccc2)c2ccc3cc4c(cc3c2)oc2cc3c(cc24)oc2cc4cc(N(c5ccccc5)c5ccc(C(C)(C)C)cc5)ccc4cc23)cc1]

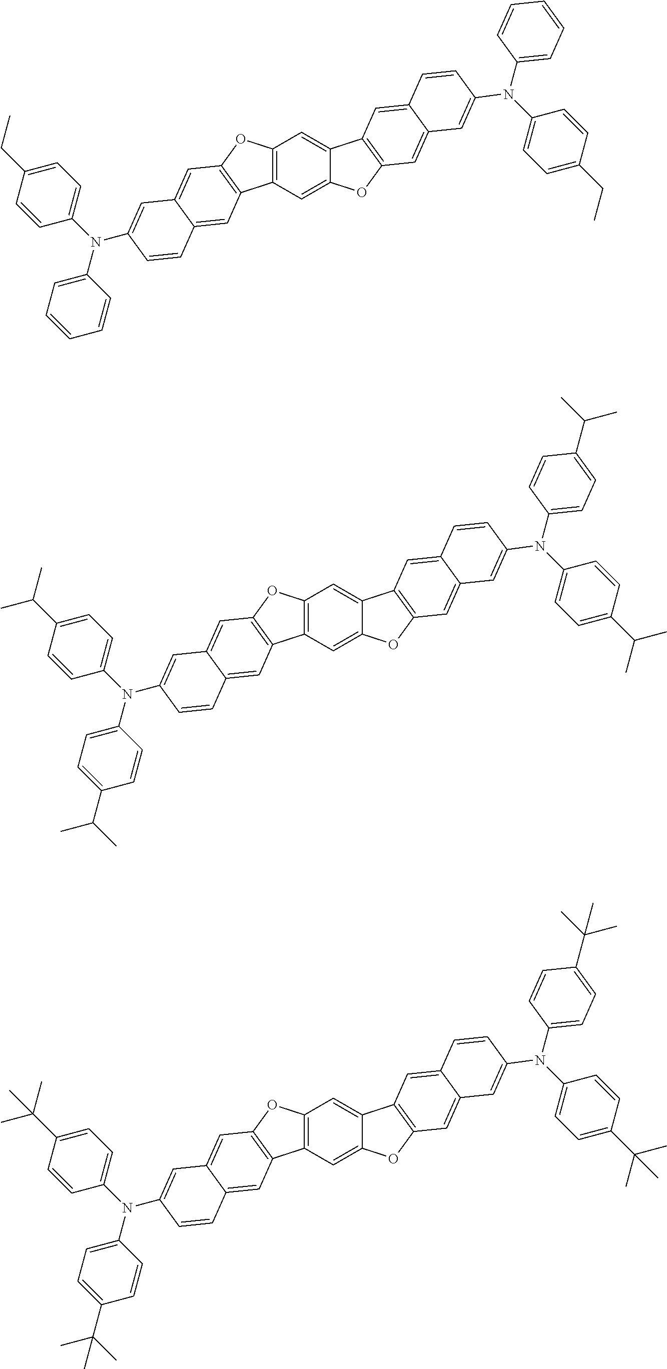

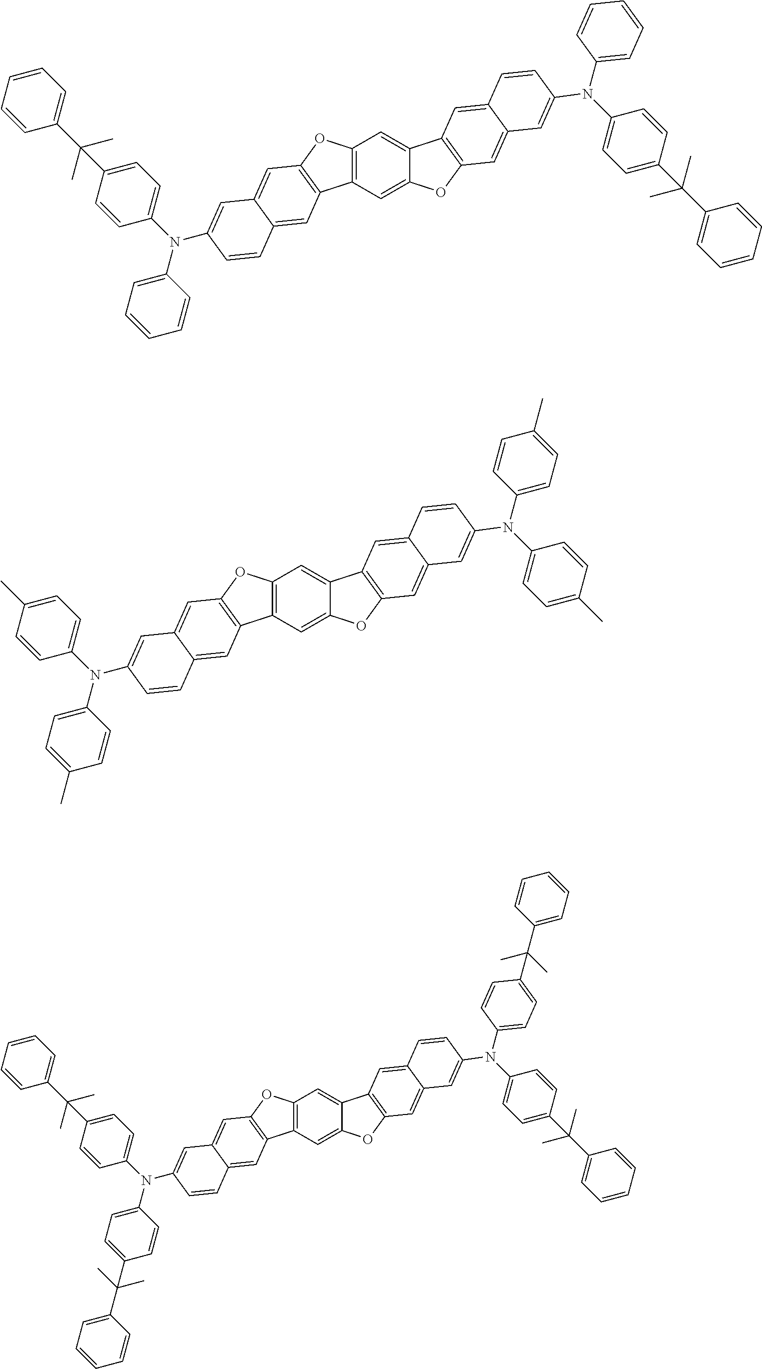

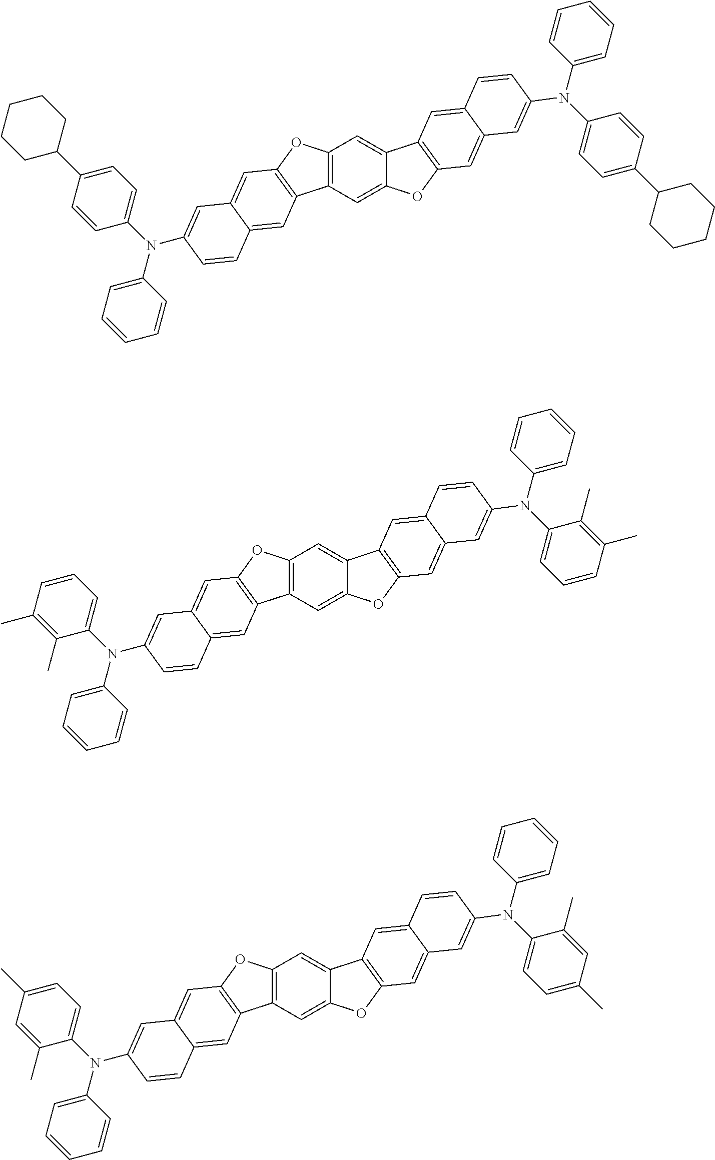

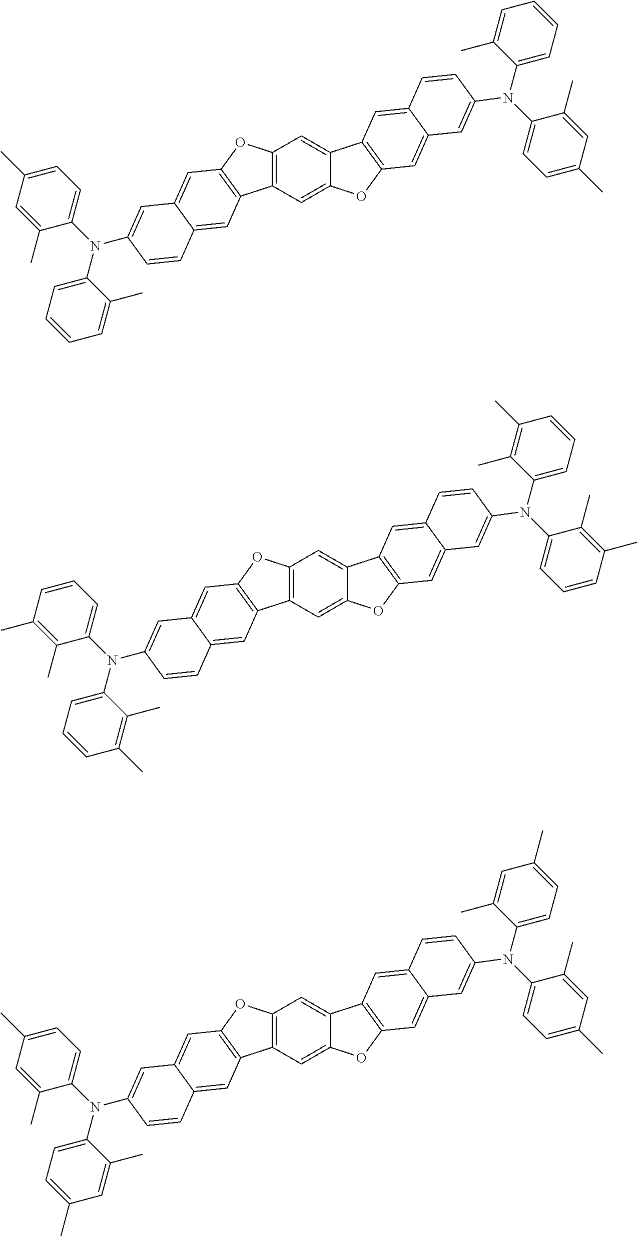

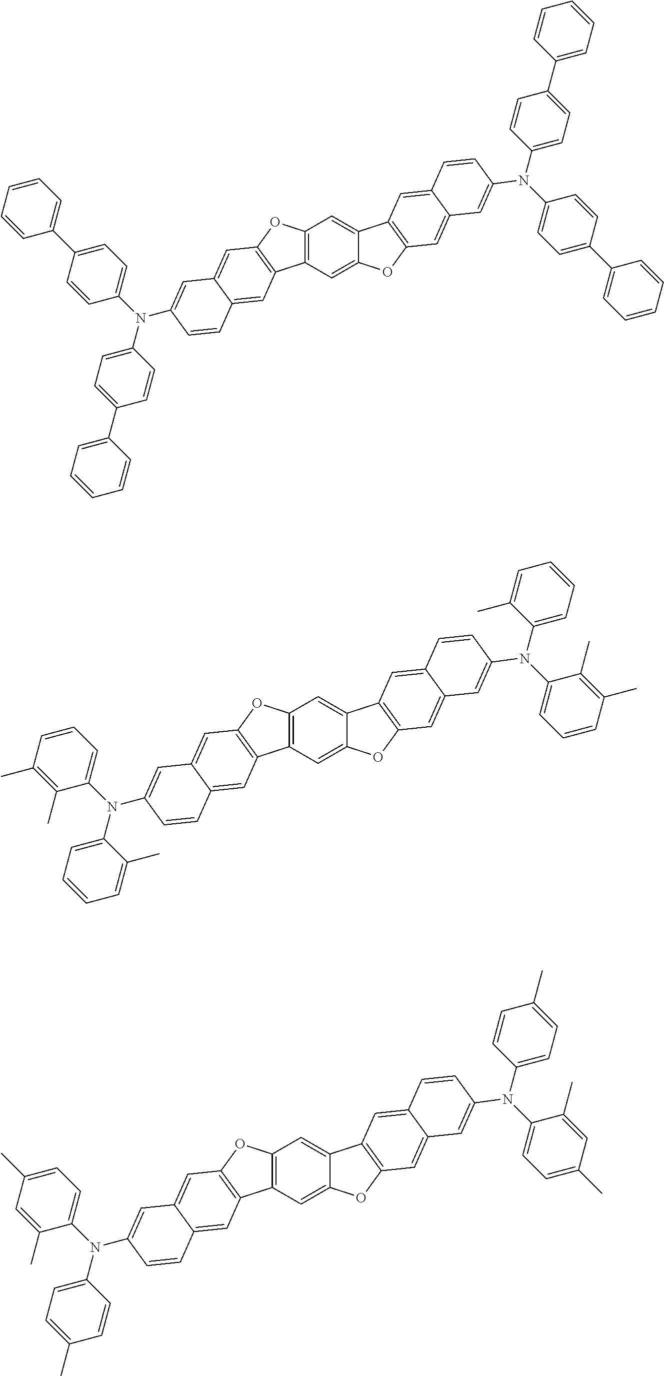

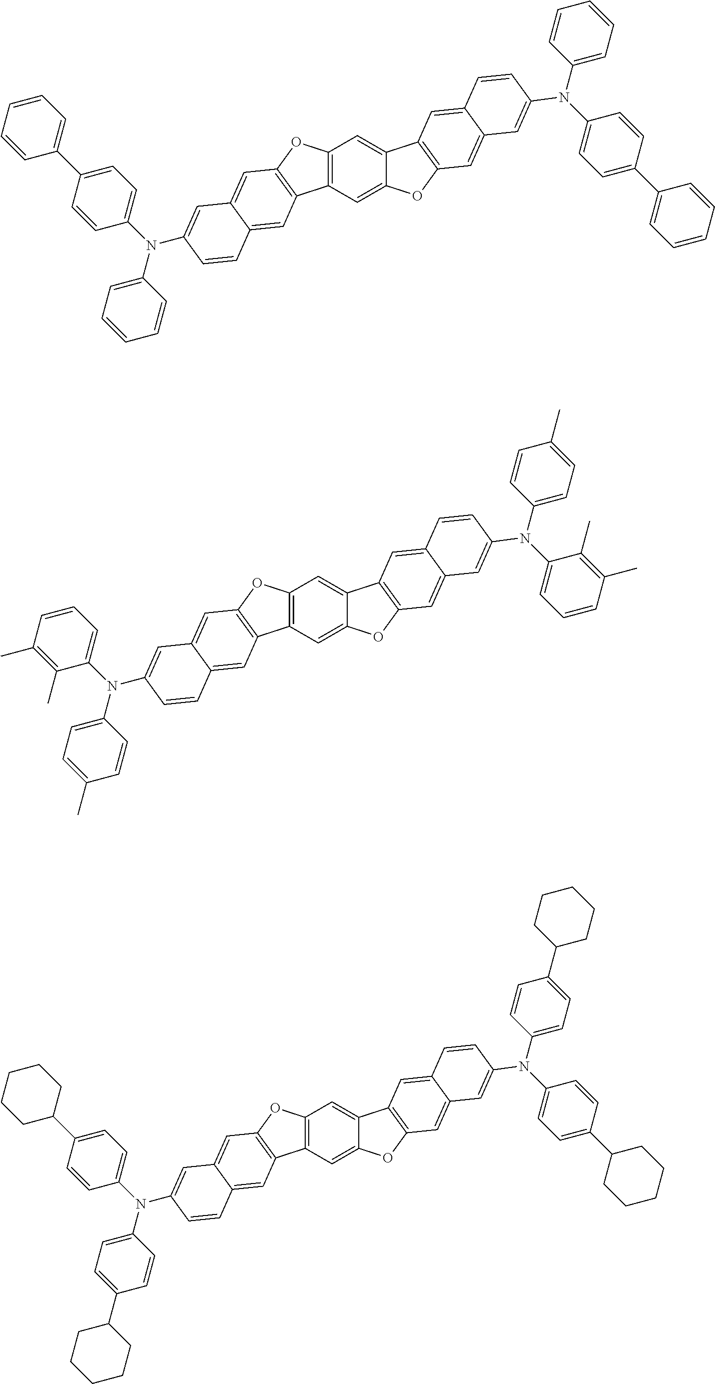

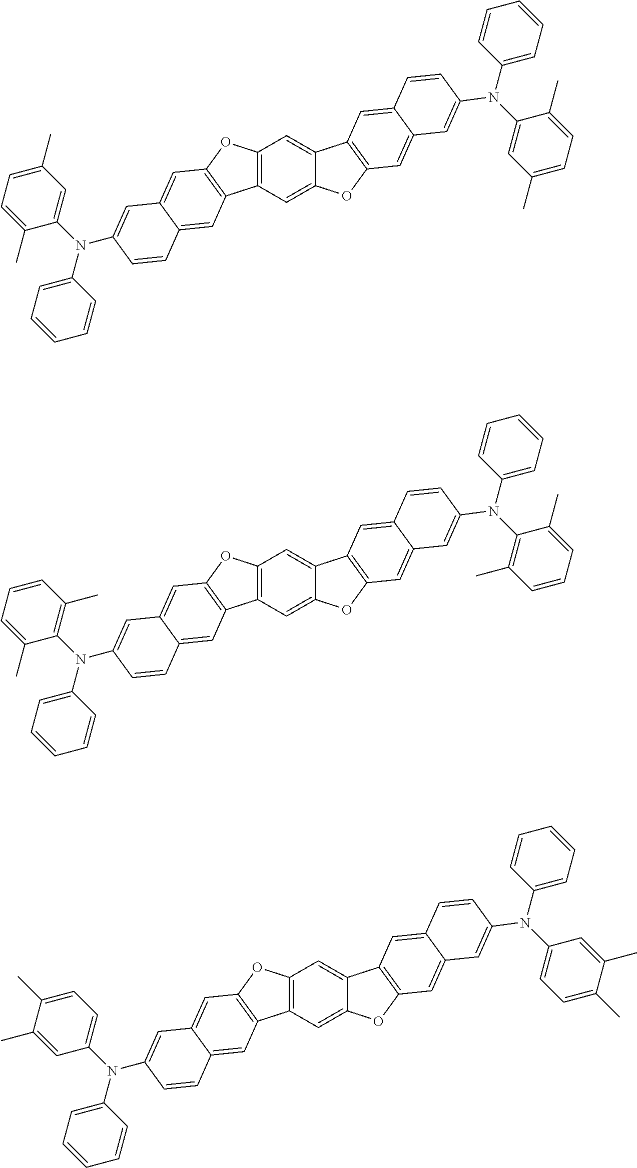

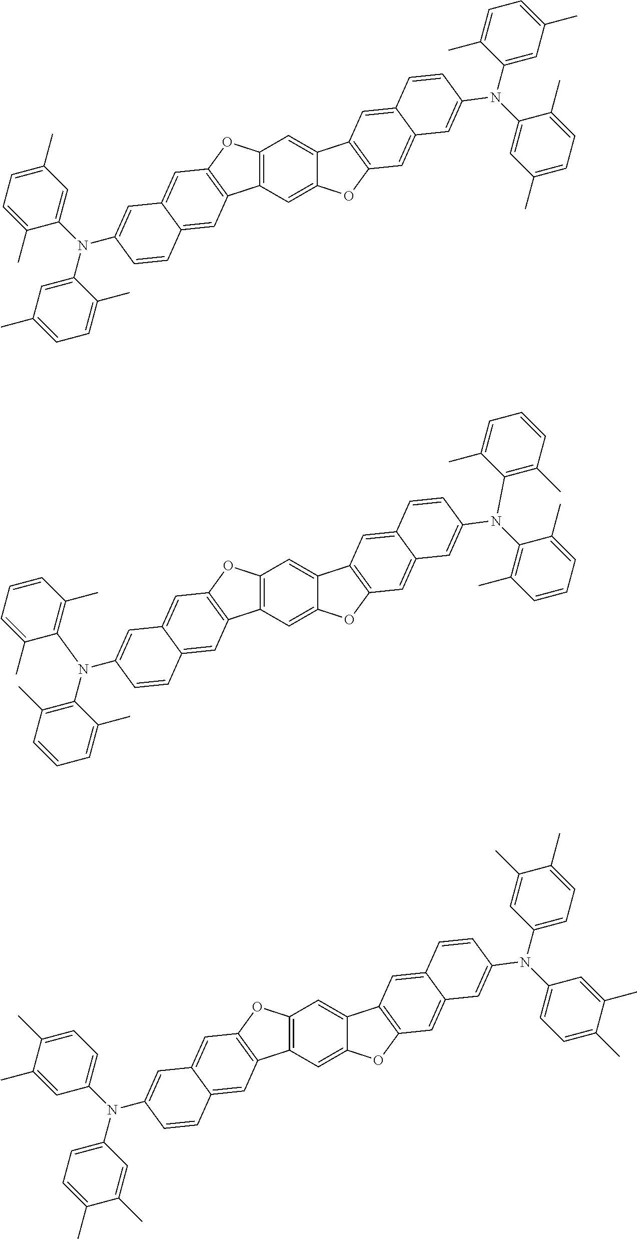

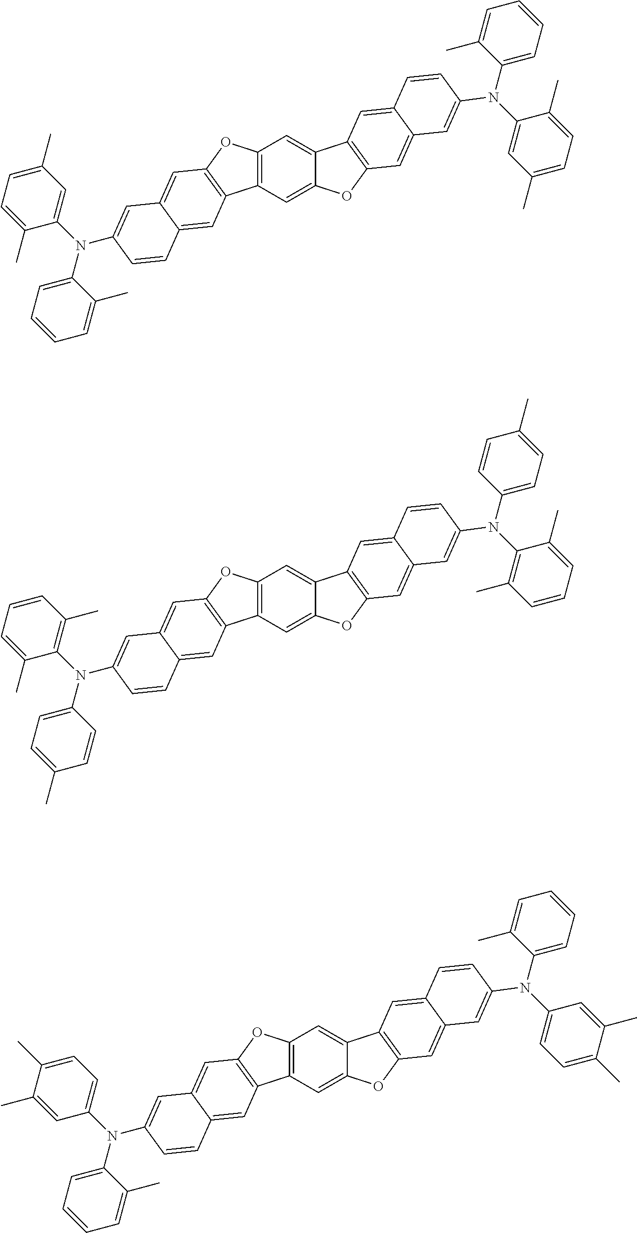

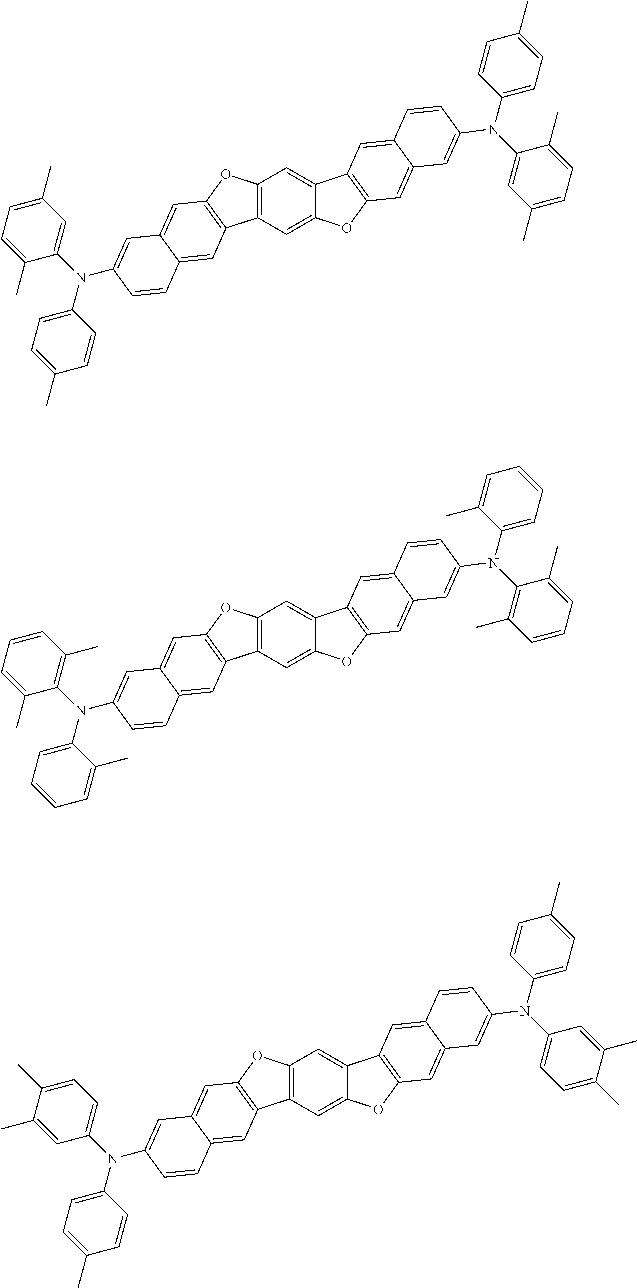

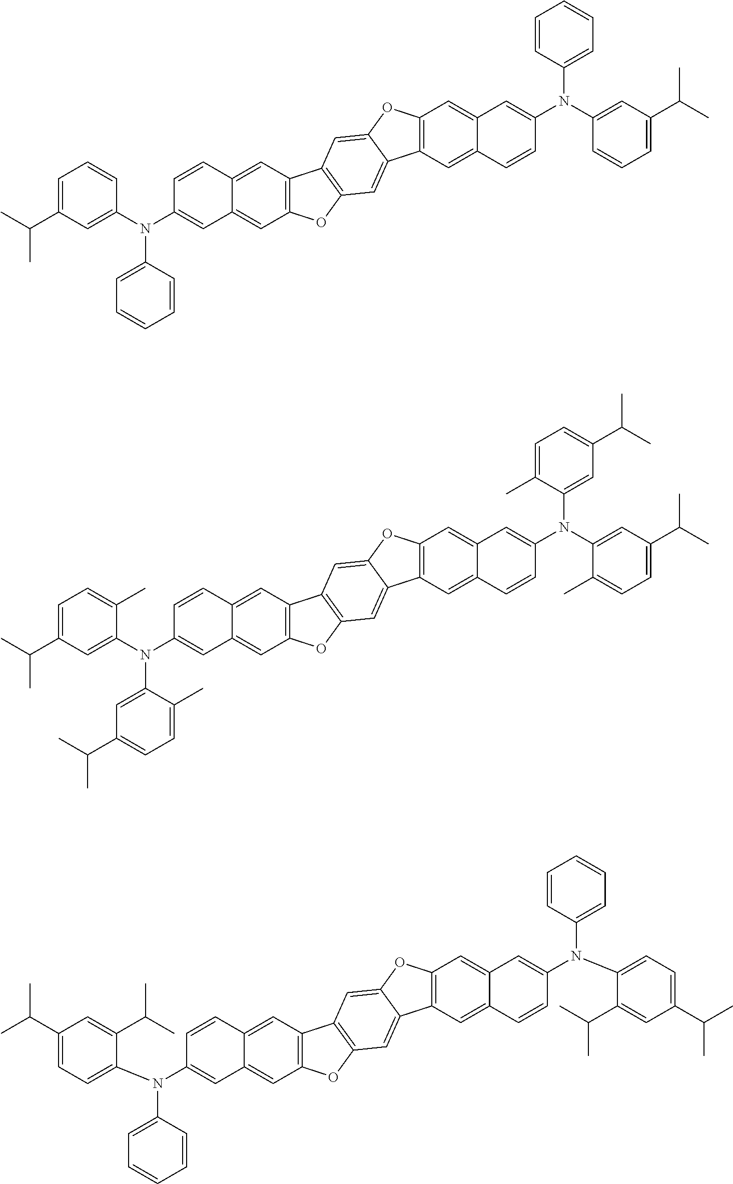

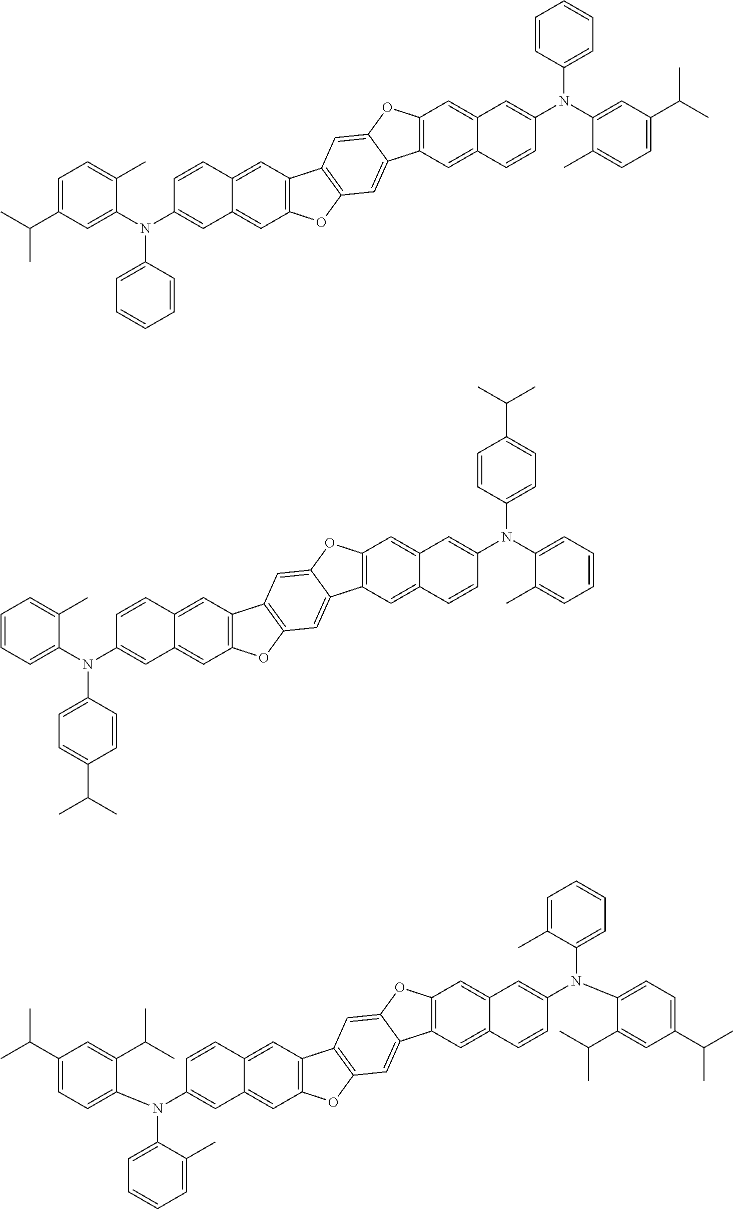

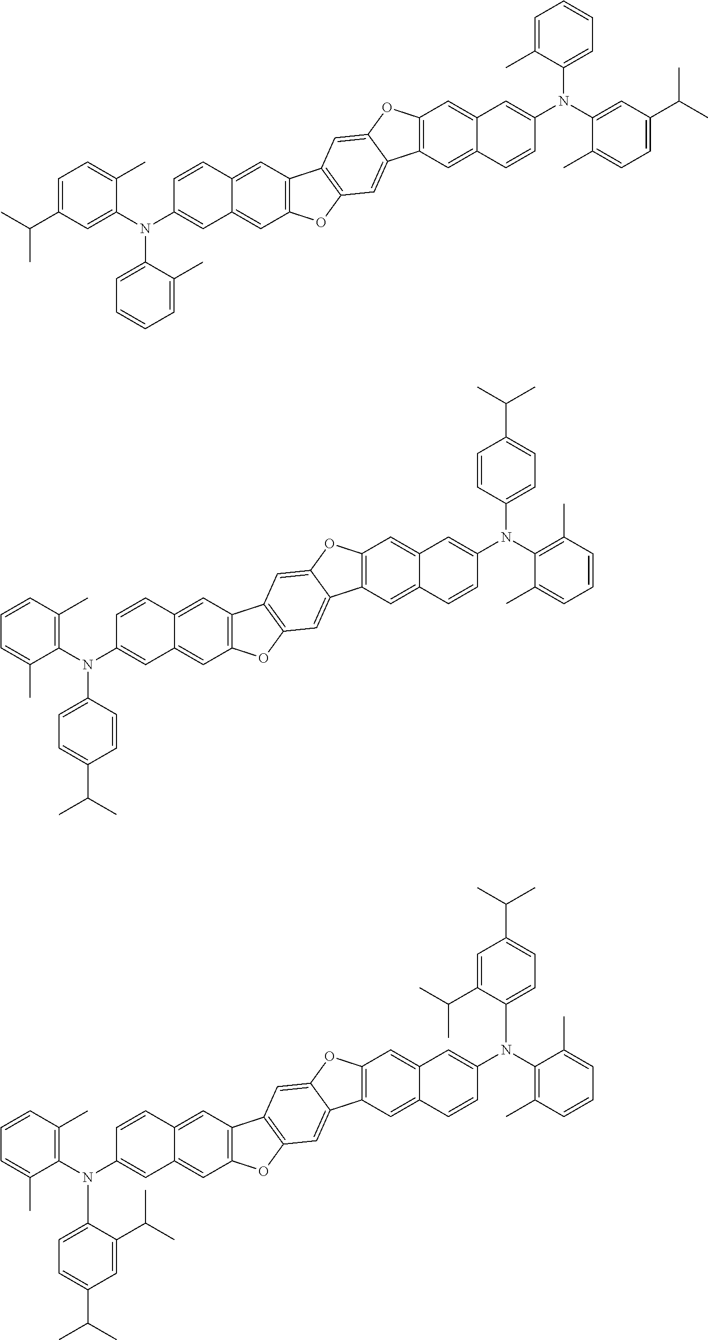 [SMILES: Cc1ccccc1N(c1ccc2cc3c(cc2c1)oc1cc2c(cc13)oc1cc3cc(N(c4ccccc4C)c4cc(C(C)C)ccc4C)ccc3cc12)c1cc(C(C)C)ccc1C ; Cc1cccc(C)c1N(c1ccc(C(C)C)cc1)c1ccc2cc3c(cc2c1)oc1cc2c(cc13)oc1cc3cc(N(c4ccc(C(C)C)cc4)c4c(C)cccc4C)ccc3cc12 ; Cc1cccc(C)c1N(c1ccc2cc3c(cc2c1)oc1cc2c(cc13)oc1cc3cc(N(c4ccc(C(C)C)cc4C(C)C)c4c(C)cccc4C)ccc3cc12)c1ccc(C(C)C)cc1C(C)C]

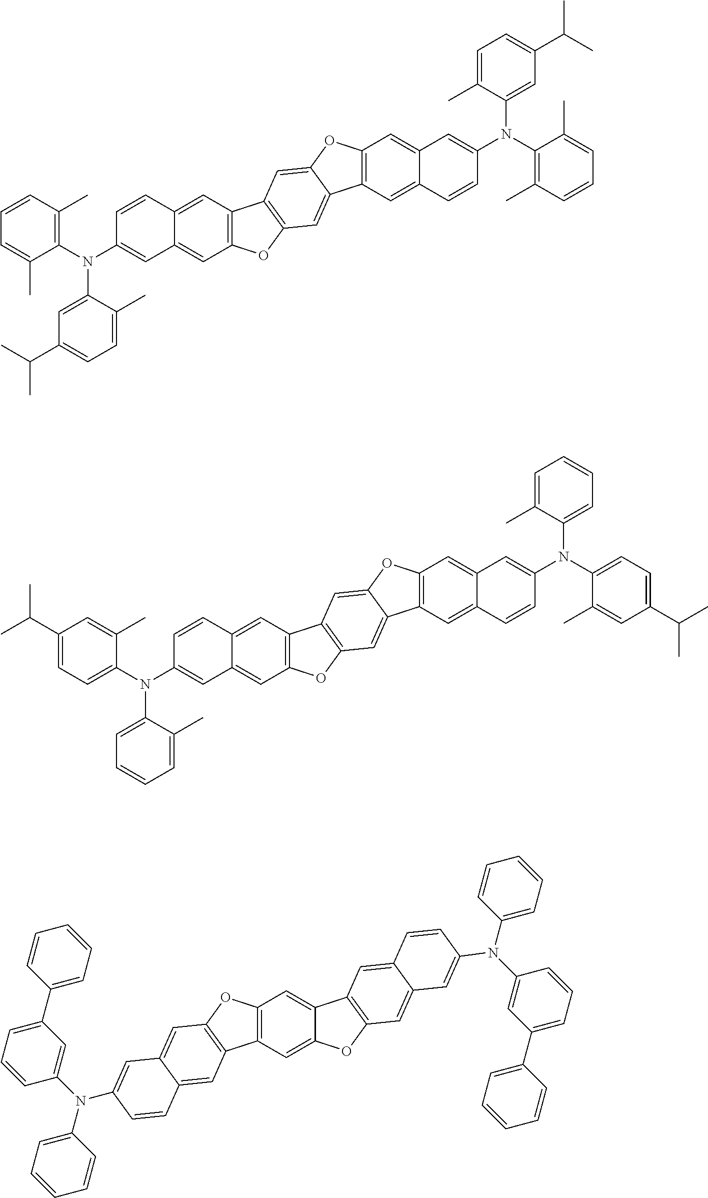

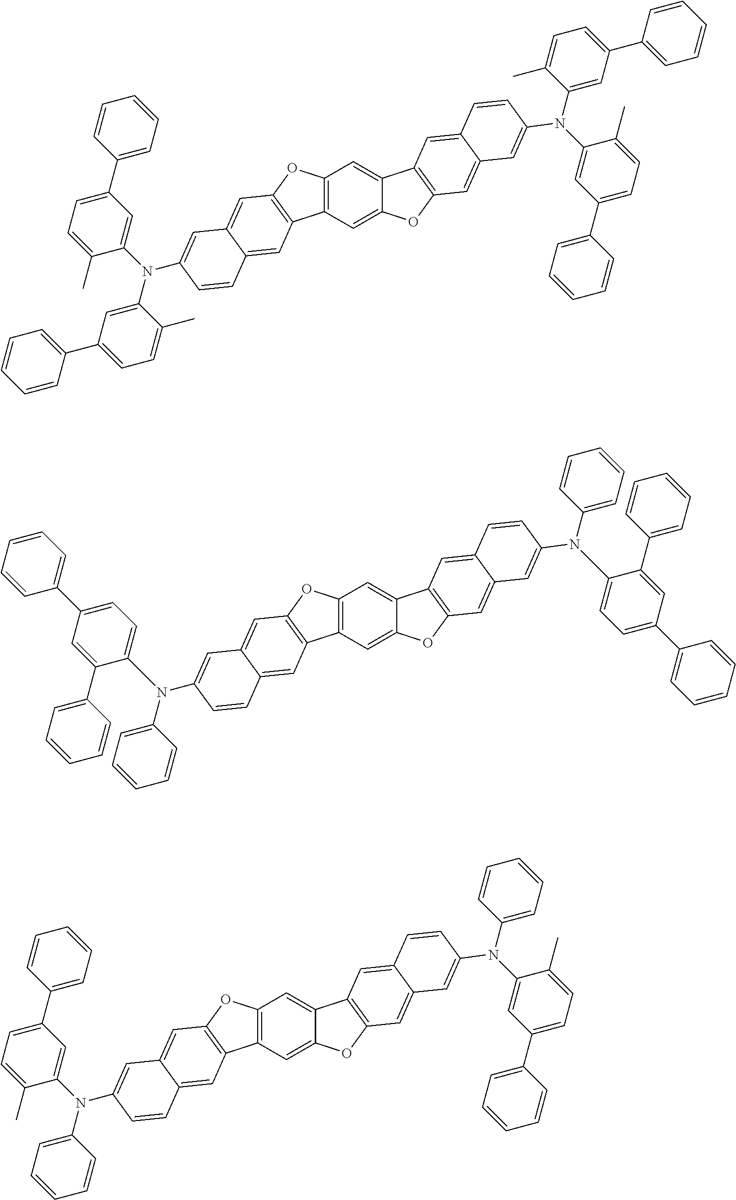 [SMILES: Cc1ccc(-c2ccccc2)cc1N(c1ccc2cc3c(cc2c1)oc1cc2c(cc13)oc1cc3cc(N(c4cc(-c5ccccc5)ccc4C)c4cc(-c5ccccc5)ccc4C)ccc3cc12)c1cc(-c2ccccc2)ccc1C ; c1ccc(-c2ccc(N(c3ccccc3)c3ccc4cc5c(cc4c3)oc3cc4c(cc35)oc3cc5cc(N(c6ccccc6)c6ccc(-c7ccccc7)cc6-c6ccccc6)ccc5cc34)c(-c3ccccc3)c2)cc1 ; Cc1ccc(-c2ccccc2)cc1N(c1ccccc1)c1ccc2cc3c(cc2c1)oc1cc2c(cc13)oc1cc3cc(N(c4ccccc4)c4cc(-c5ccccc5)ccc4C)ccc3cc12]

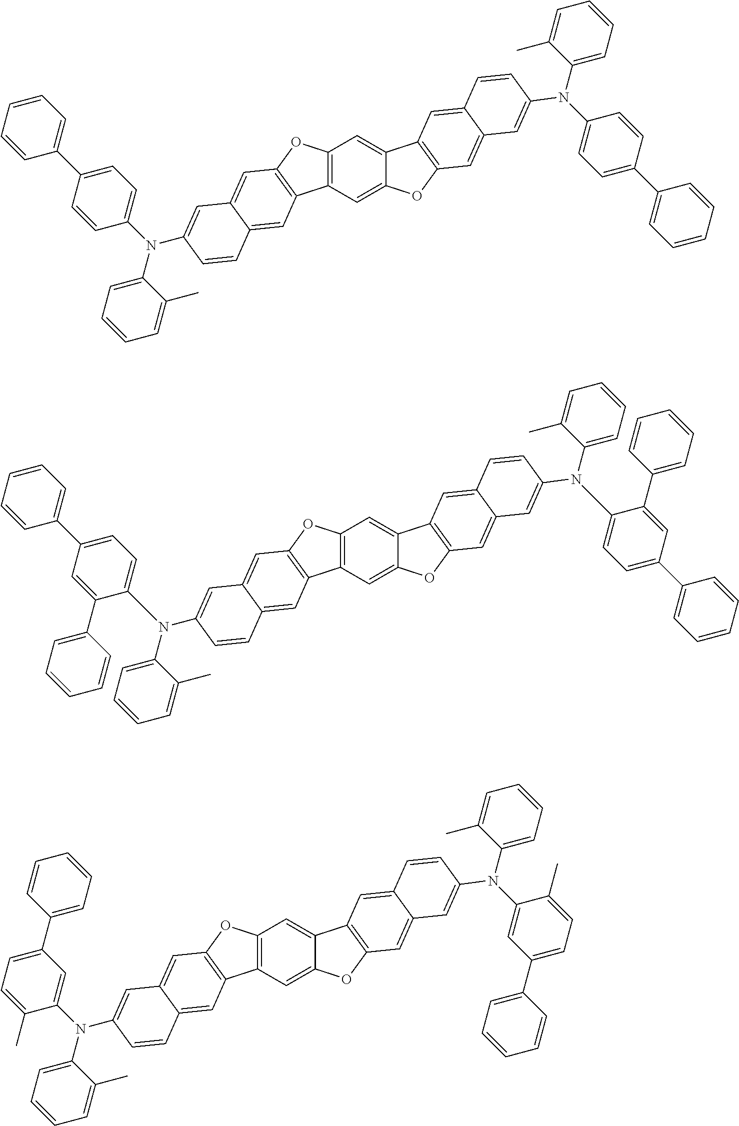 [SMILES: Cc1ccccc1N(c1ccc(-c2ccccc2)cc1)c1ccc2cc3c(cc2c1)oc1cc2c(cc13)oc1cc3cc(N(c4ccc(-c5ccccc5)cc4)c4ccccc4C)ccc3cc12 ; Cc1ccccc1N(c1ccc2cc3c(cc2c1)oc1cc2c(cc13)oc1cc3cc(N(c4ccccc4C)c4ccc(-c5ccccc5)cc4-c4ccccc4)ccc3cc12)c1ccc(-c2ccccc2)cc1-c1ccccc1 ; Cc1ccccc1N(c1ccc2cc3c(cc2c1)oc1cc2c(cc13)oc1cc3cc(N(c4ccccc4C)c4cc(-c5ccccc5)ccc4C)ccc3cc12)c1cc(-c2ccccc2)ccc1C]

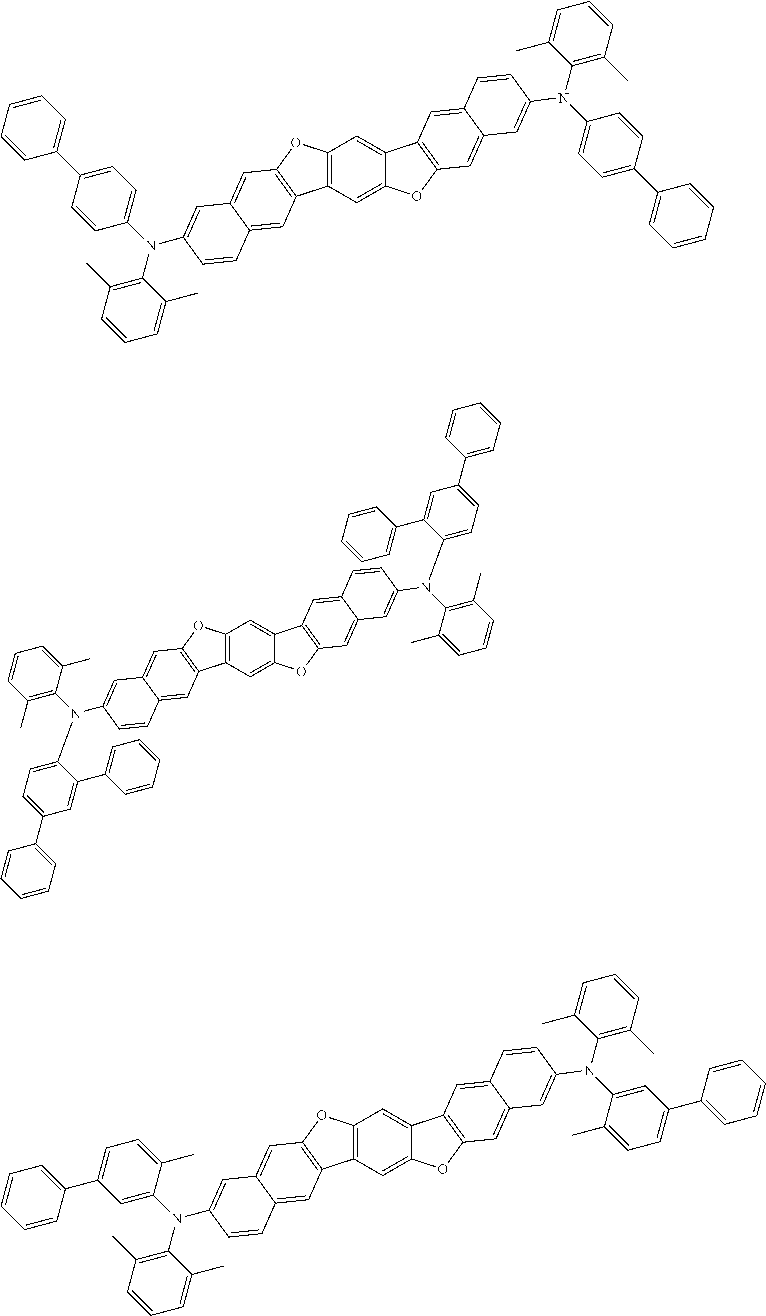 [SMILES: Cc1cccc(C)c1N(c1ccc(-c2ccccc2)cc1)c1ccc2cc3c(cc2c1)oc1cc2c(cc13)oc1cc3cc(N(c4ccc(-c5ccccc5)cc4)c4c(C)cccc4C)ccc3cc12 ; Cc1cccc(C)c1N(c1ccc2cc3c(cc2c1)oc1cc2c(cc13)oc1cc3cc(N(c4ccc(-c5ccccc5)cc4-c4ccccc4)c4c(C)cccc4C)ccc3cc12)c1ccc(-c2ccccc2)cc1-c1ccccc1 ; Cc1ccc(-c2ccccc2)cc1N(c1ccc2cc3c(cc2c1)oc1cc2c(cc13)oc1cc3cc(N(c4cc(-c5ccccc5)ccc4C)c4c(C)cccc4C)ccc3cc12)c1c(C)cccc1C]

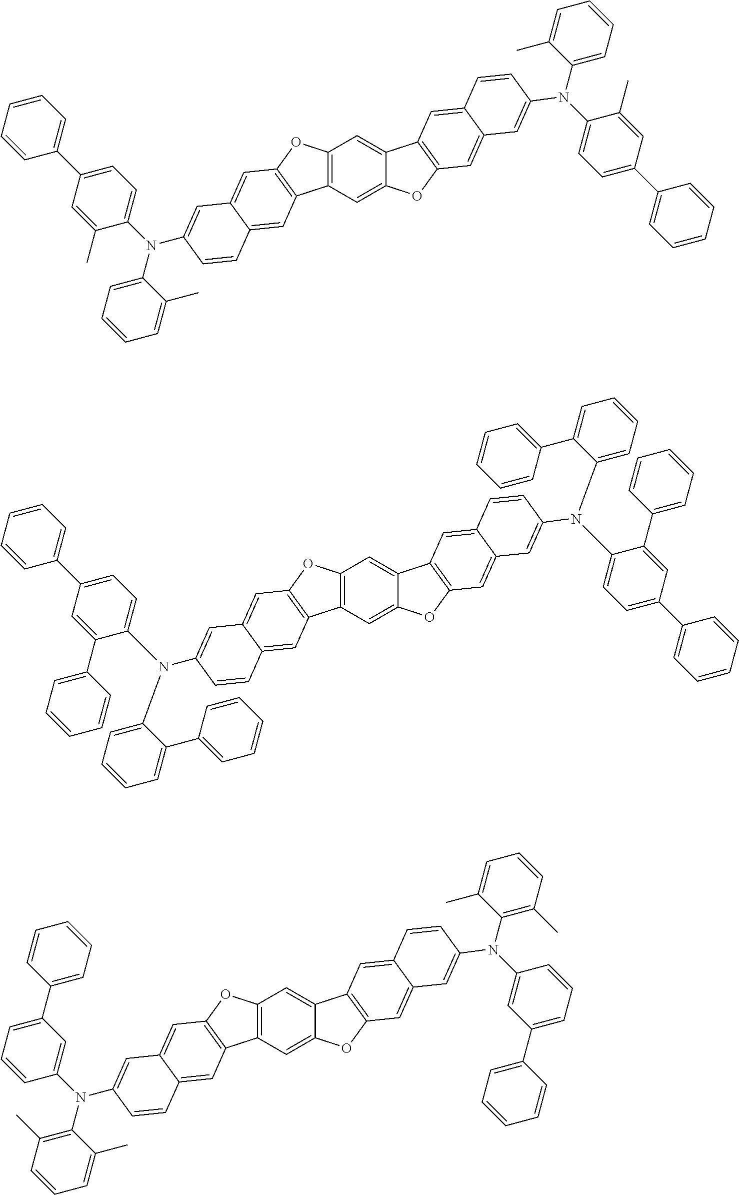 [SMILES: Cc1ccccc1N(c1ccc2cc3c(cc2c1)oc1cc2c(cc13)oc1cc3cc(N(c4ccccc4C)c4ccc(-c5ccccc5)cc4C)ccc3cc12)c1ccc(-c2ccccc2)cc1C ; c1ccc(-c2ccc(N(c3ccc4cc5c(cc4c3)oc3cc4c(cc35)oc3cc5cc(N(c6ccccc6-c6ccccc6)c6ccc(-c7ccccc7)cc6-c6ccccc6)ccc5cc34)c3ccccc3-c3ccccc3)c(-c3ccccc3)c2)cc1 ; Cc1cccc(C)c1N(c1cccc(-c2ccccc2)c1)c1ccc2cc3c(cc2c1)oc1cc2c(cc13)oc1cc3cc(N(c4cccc(-c5ccccc5)c4)c4c(C)cccc4C)ccc3cc12]

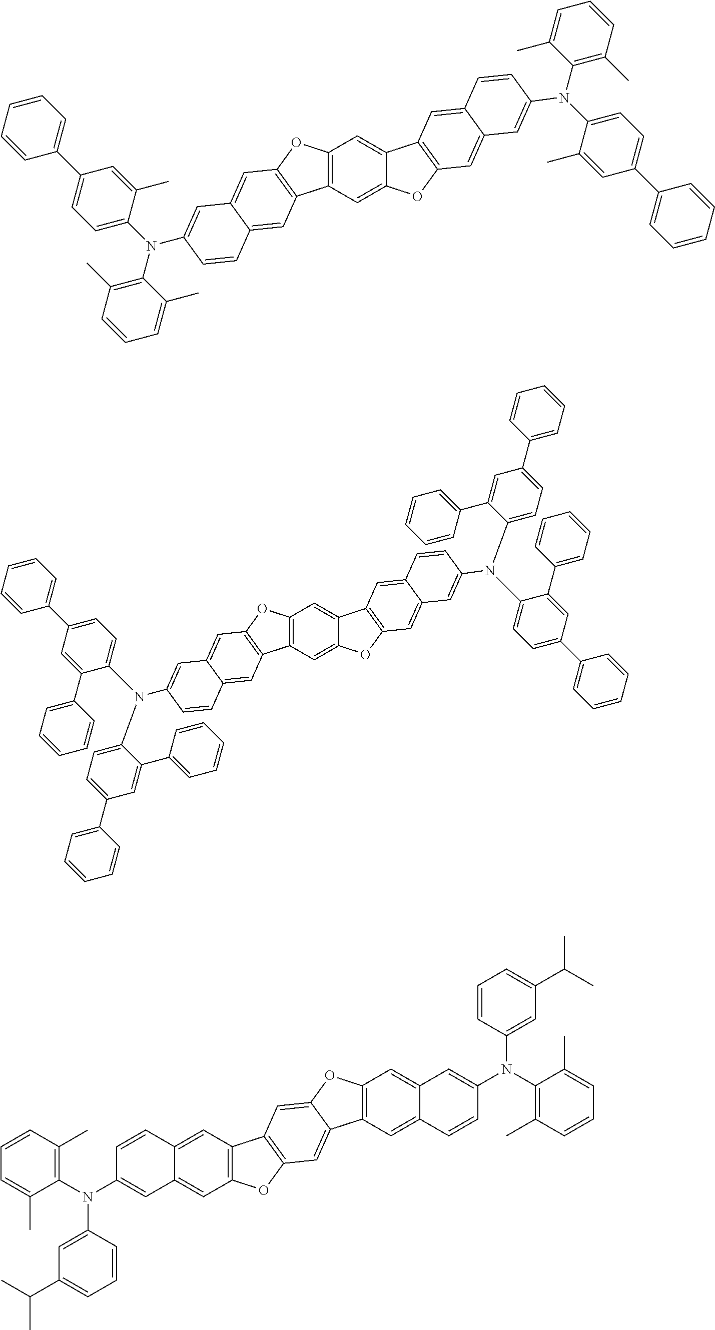 [SMILES: Cc1cc(-c2ccccc2)ccc1N(c1ccc2cc3c(cc2c1)oc1cc2c(cc13)oc1cc3cc(N(c4ccc(-c5ccccc5)cc4C)c4c(C)cccc4C)ccc3cc12)c1c(C)cccc1C ; c1ccc(-c2ccc(N(c3ccc4cc5c(cc4c3)oc3cc4c(cc35)oc3cc5cc(N(c6ccc(-c7ccccc7)cc6-c6ccccc6)c6ccc(-c7ccccc7)cc6-c6ccccc6)ccc5cc34)c3ccc(-c4ccccc4)cc3-c3ccccc3)c(-c3ccccc3)c2)cc1 ; Cc1cccc(C)c1N(c1cccc(C(C)C)c1)c1ccc2cc3c(cc2c1)oc1cc2c(cc13)oc1cc3cc(N(c4cccc(C(C)C)c4)c4c(C)cccc4C)ccc3cc12]

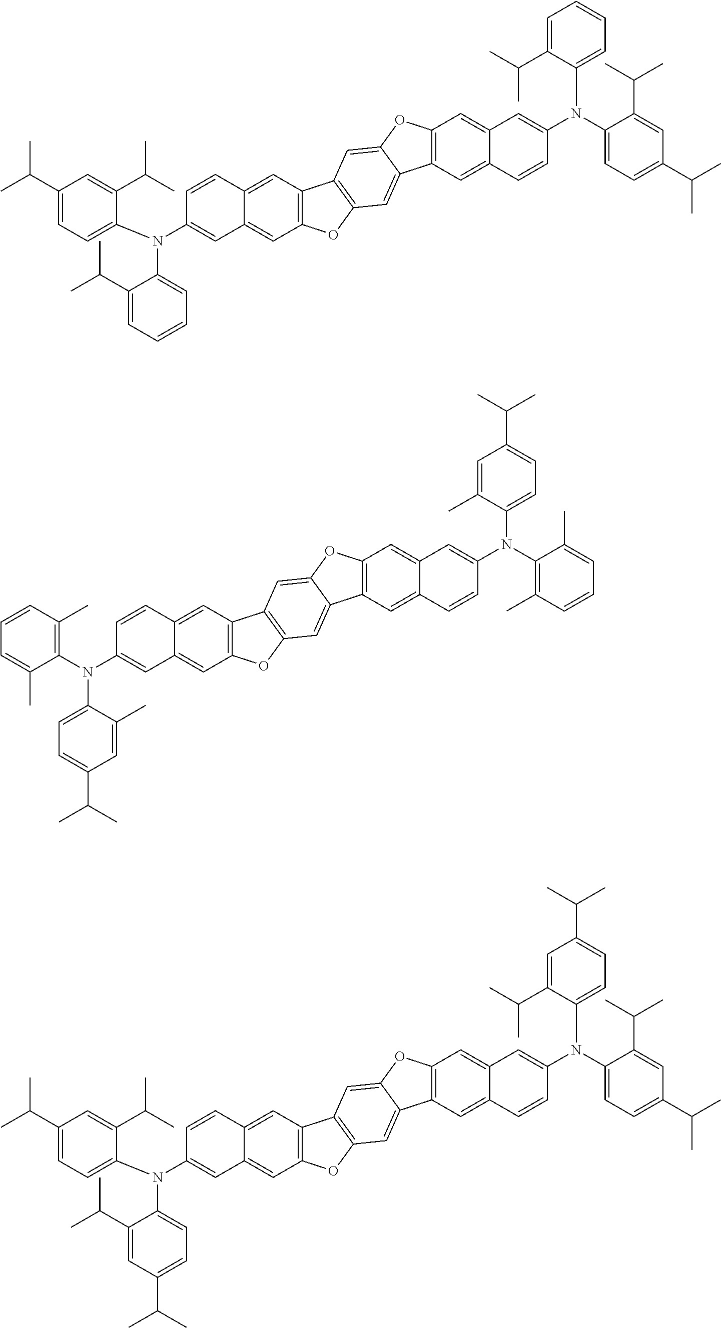

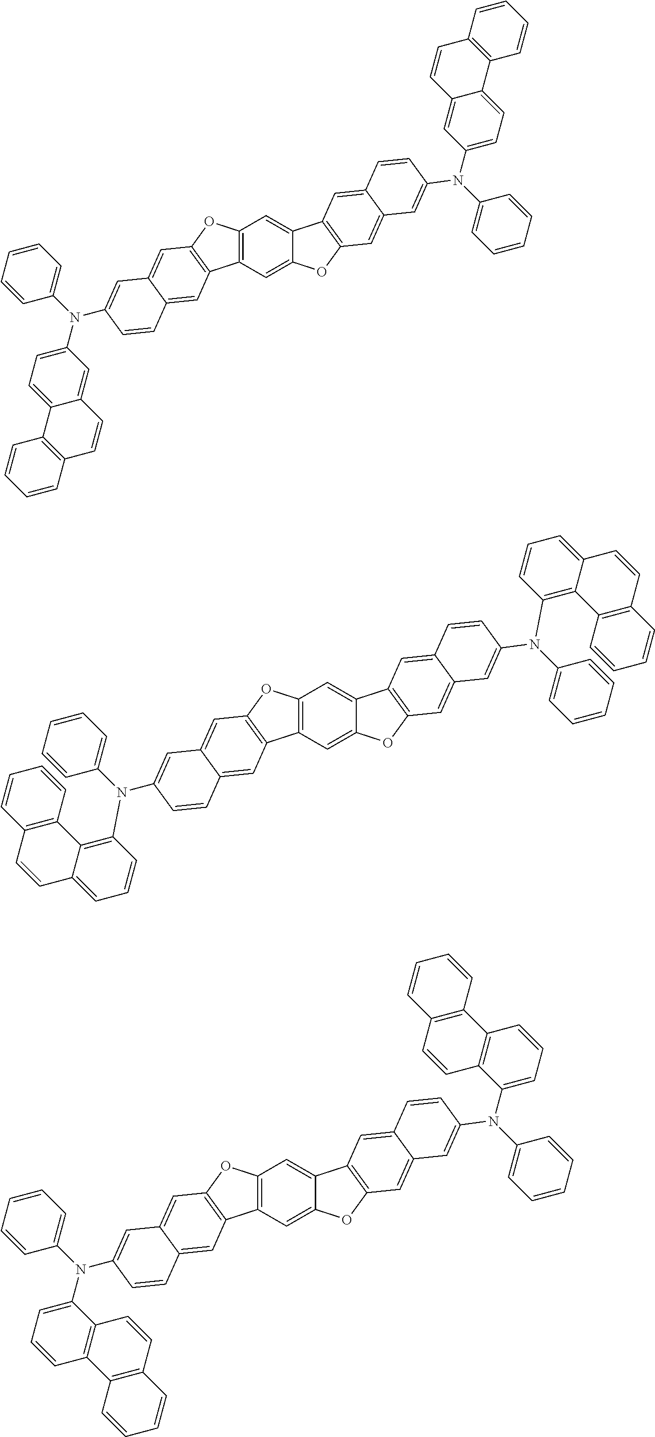 [SMILES: c1ccc(N(c2ccc3cc4c(cc3c2)oc2cc3c(cc24)oc2cc4cc(N(c5ccccc5)c5ccc6c(ccc7ccccc76)c5)ccc4cc23)c2ccc3c(ccc4ccccc43)c2)cc1 ; c1ccc(N(c2ccc3cc4c(cc3c2)oc2cc3c(cc24)oc2cc4cc(N(c5ccccc5)c5cccc6ccc7ccccc7c56)ccc4cc23)c2cccc3ccc4ccccc4c23)cc1 ; c1ccc(N(c2ccc3cc4c(cc3c2)oc2cc3c(cc24)oc2cc4cc(N(c5ccccc5)c5cccc6c5ccc5ccccc56)ccc4cc23)c2cccc3c2ccc2ccccc23)cc1]

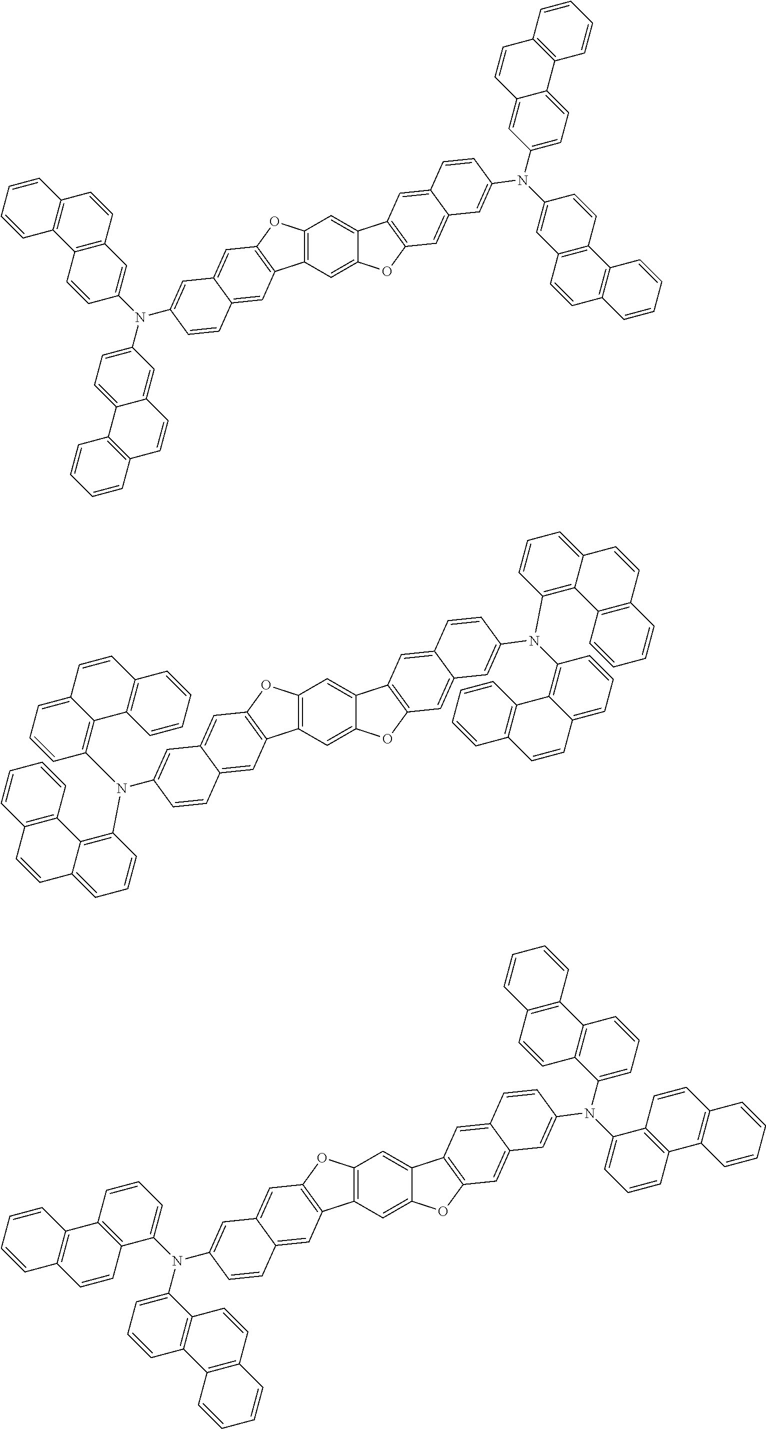 [SMILES: c1ccc2c(c1)ccc1cc(N(c3ccc4cc5c(cc4c3)oc3cc4c(cc35)oc3cc5cc(N(c6ccc7c(ccc8ccccc87)c6)c6ccc7c(ccc8ccccc87)c6)ccc5cc34)c3ccc4c(ccc5ccccc54)c3)ccc12 ; c1ccc2c(c1)ccc1cccc(N(c3ccc4cc5c(cc4c3)oc3cc4c(cc35)oc3cc5cc(N(c6cccc7ccc8ccccc8c67)c6cccc7ccc8ccccc8c67)ccc5cc34)c3cccc4ccc5ccccc5c34)c12 ; c1ccc2c(c1)ccc1c(N(c3ccc4cc5c(cc4c3)oc3cc4c(cc35)oc3cc5cc(N(c6cccc7c6ccc6ccccc67)c6cccc7c6ccc6ccccc67)ccc5cc34)c3cccc4c3ccc3ccccc34)cccc12]

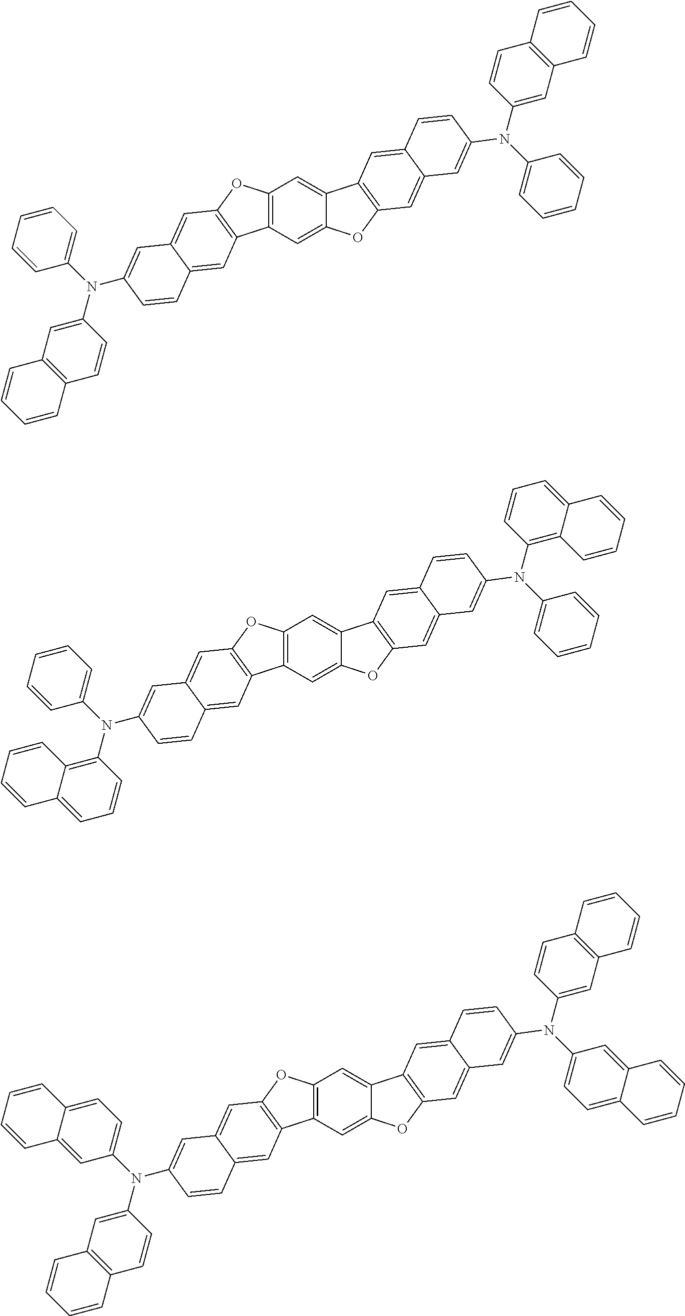 [SMILES: c1ccc(N(c2ccc3ccccc3c2)c2ccc3cc4c(cc3c2)oc2cc3c(cc24)oc2cc4cc(N(c5ccccc5)c5ccc6ccccc6c5)ccc4cc23)cc1 ; c1ccc(N(c2ccc3cc4c(cc3c2)oc2cc3c(cc24)oc2cc4cc(N(c5ccccc5)c5cccc6ccccc56)ccc4cc23)c2cccc3ccccc23)cc1 ; c1ccc2cc(N(c3ccc4ccccc4c3)c3ccc4cc5c(cc4c3)oc3cc4c(cc35)oc3cc5cc(N(c6ccc7ccccc7c6)c6ccc7ccccc7c6)ccc5cc34)ccc2c1]

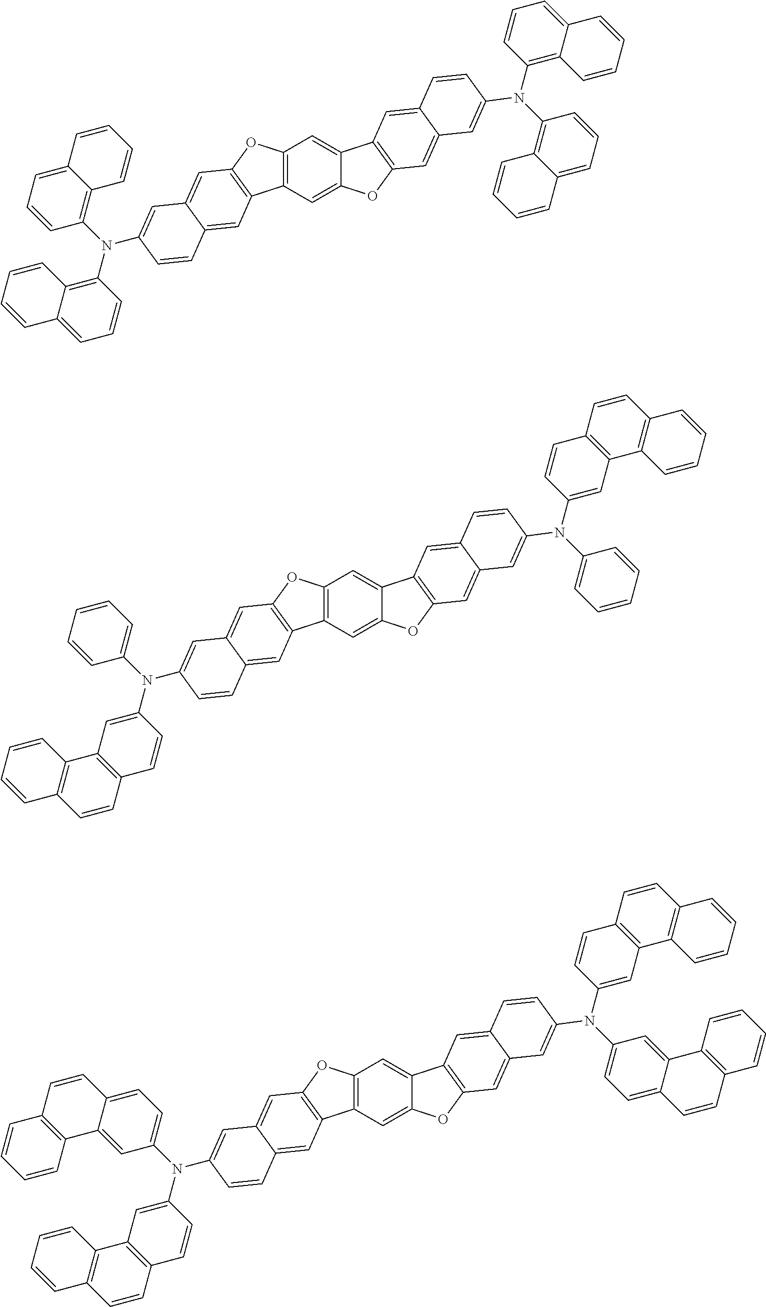 [SMILES: c1ccc2c(N(c3ccc4cc5c(cc4c3)oc3cc4c(cc35)oc3cc5cc(N(c6cccc7ccccc67)c6cccc7ccccc67)ccc5cc34)c3cccc4ccccc34)cccc2c1 ; c1ccc(N(c2ccc3cc4c(cc3c2)oc2cc3c(cc24)oc2cc4cc(N(c5ccccc5)c5ccc6ccc7ccccc7c6c5)ccc4cc23)c2ccc3ccc4ccccc4c3c2)cc1 ; c1ccc2c(c1)ccc1ccc(N(c3ccc4cc5c(cc4c3)oc3cc4c(cc35)oc3cc5cc(N(c6ccc7ccc8ccccc8c7c6)c6ccc7ccc8ccccc8c7c6)ccc5cc34)c3ccc4ccc5ccccc5c4c3)cc12]

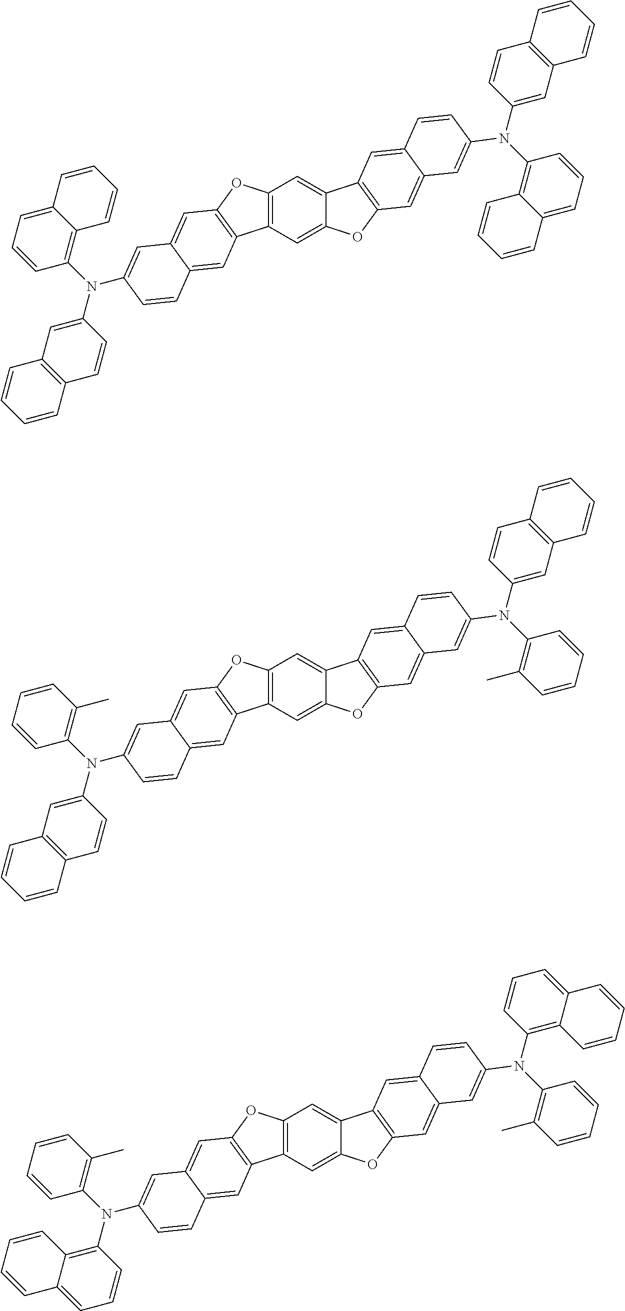 [SMILES: c1ccc2cc(N(c3ccc4cc5c(cc4c3)oc3cc4c(cc35)oc3cc5cc(N(c6ccc7ccccc7c6)c6cccc7ccccc67)ccc5cc34)c3cccc4ccccc34)ccc2c1 ; Cc1ccccc1N(c1ccc2ccccc2c1)c1ccc2cc3c(cc2c1)oc1cc2c(cc13)oc1cc3cc(N(c4ccc5ccccc5c4)c4ccccc4C)ccc3cc12 ; Cc1ccccc1N(c1ccc2cc3c(cc2c1)oc1cc2c(cc13)oc1cc3cc(N(c4ccccc4C)c4cccc5ccccc45)ccc3cc12)c1cccc2ccccc12]

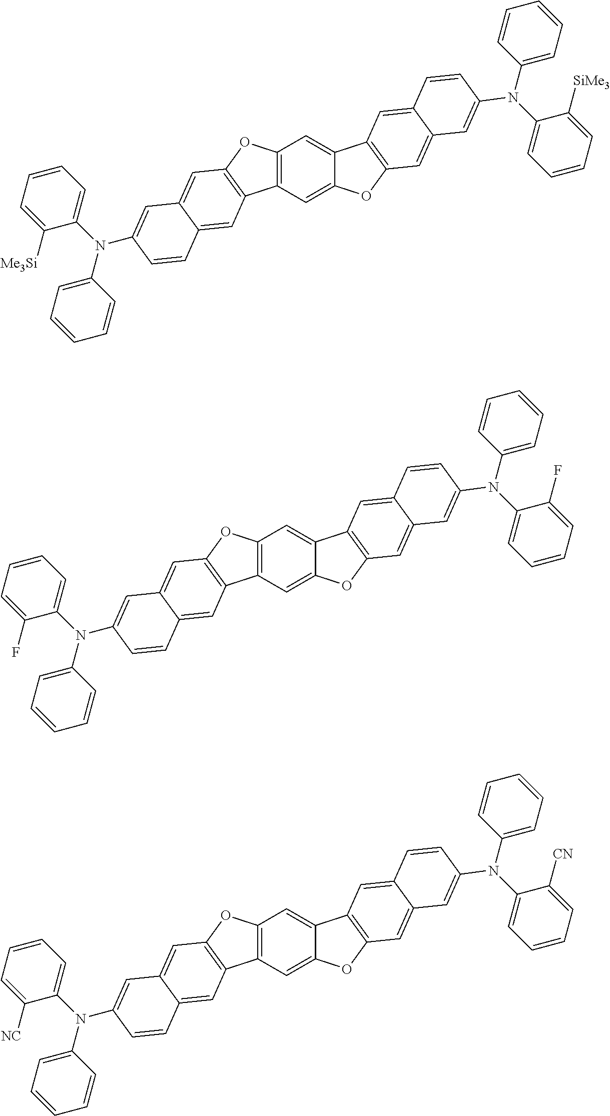 [SMILES: C[Si](C)(C)c1ccccc1N(c1ccccc1)c1ccc2cc3c(cc2c1)oc1cc2c(cc13)oc1cc3cc(N(c4ccccc4)c4ccccc4[Si](C)(C)C)ccc3cc12 ; Fc1ccccc1N(c1ccccc1)c1ccc2cc3c(cc2c1)oc1cc2c(cc13)oc1cc3cc(N(c4ccccc4)c4ccccc4F)ccc3cc12 ; N#Cc1ccccc1N(c1ccccc1)c1ccc2cc3c(cc2c1)oc1cc2c(cc13)oc1cc3cc(N(c4ccccc4)c4ccccc4C#N)ccc3cc12]

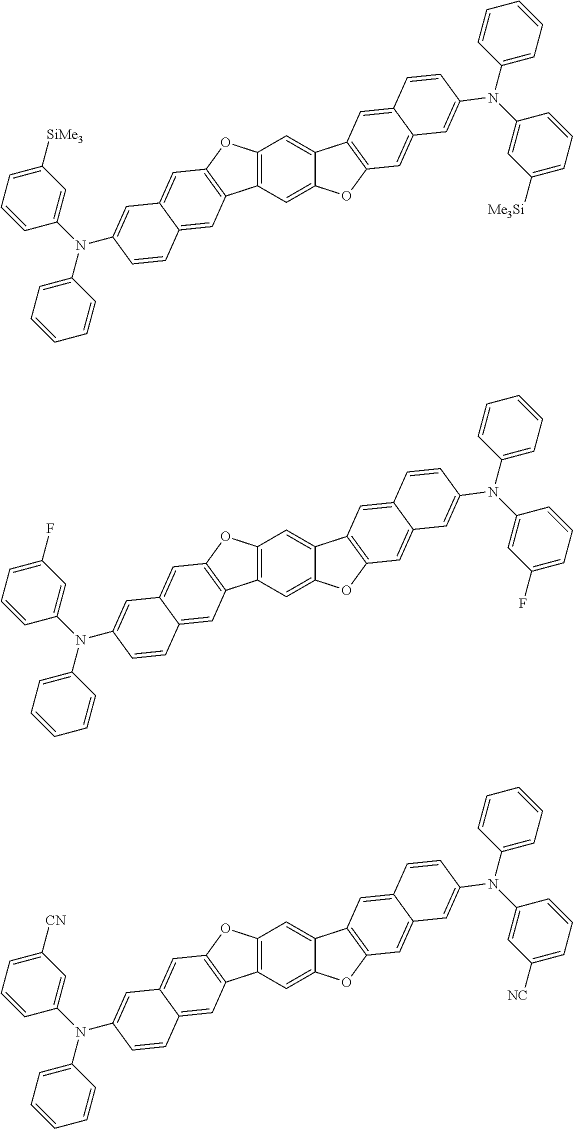 [SMILES: C[Si](C)(C)c1cccc(N(c2ccccc2)c2ccc3cc4c(cc3c2)oc2cc3c(cc24)oc2cc4cc(N(c5ccccc5)c5cccc([Si](C)(C)C)c5)ccc4cc23)c1 ; Fc1cccc(N(c2ccccc2)c2ccc3cc4c(cc3c2)oc2cc3c(cc24)oc2cc4cc(N(c5ccccc5)c5cccc(F)c5)ccc4cc23)c1 ; N#Cc1cccc(N(c2ccccc2)c2ccc3cc4c(cc3c2)oc2cc3c(cc24)oc2cc4cc(N(c5ccccc5)c5cccc(C#N)c5)ccc4cc23)c1]

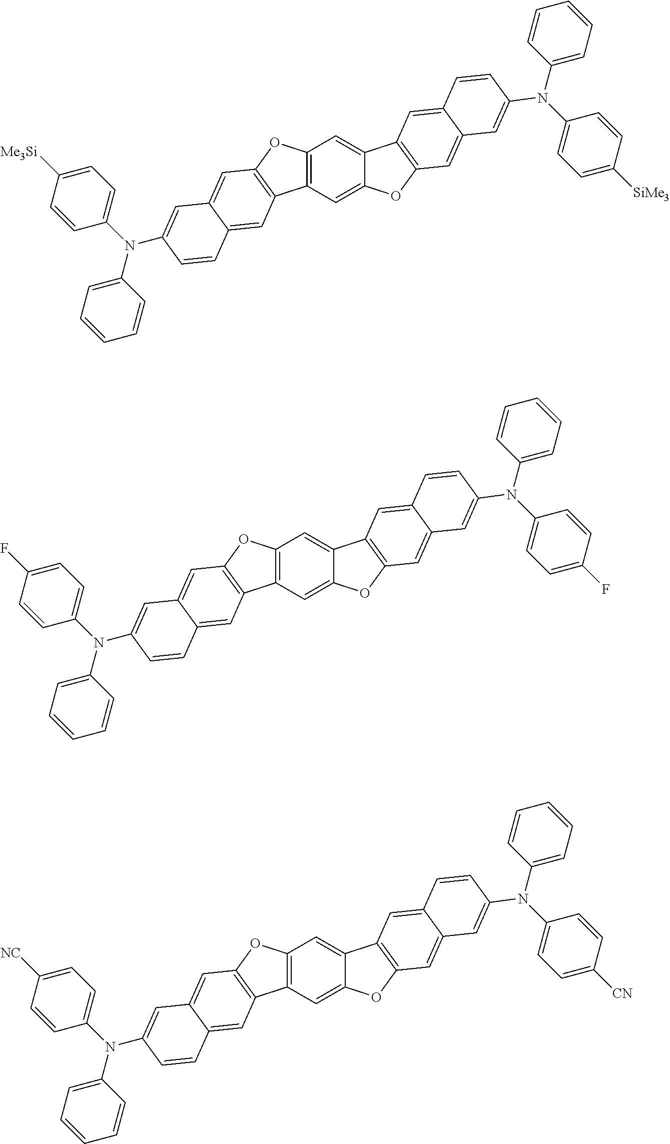 [SMILES: C[Si](C)(C)c1ccc(N(c2ccccc2)c2ccc3cc4c(cc3c2)oc2cc3c(cc24)oc2cc4cc(N(c5ccccc5)c5ccc([Si](C)(C)C)cc5)ccc4cc23)cc1 ; Fc1ccc(N(c2ccccc2)c2ccc3cc4c(cc3c2)oc2cc3c(cc24)oc2cc4cc(N(c5ccccc5)c5ccc(F)cc5)ccc4cc23)cc1 ; N#Cc1ccc(N(c2ccccc2)c2ccc3cc4c(cc3c2)oc2cc3c(cc24)oc2cc4cc(N(c5ccccc5)c5ccc(C#N)cc5)ccc4cc23)cc1]

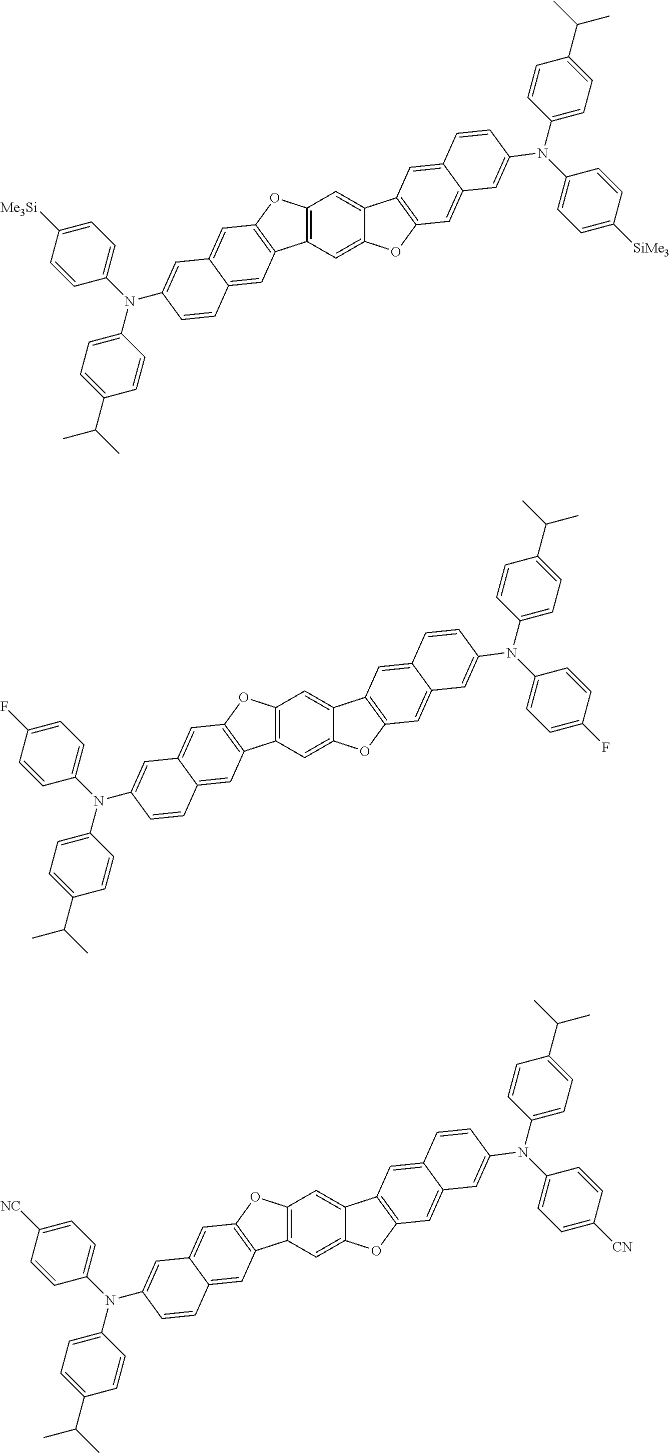 [SMILES: CC(C)c1ccc(N(c2ccc([Si](C)(C)C)cc2)c2ccc3cc4c(cc3c2)oc2cc3c(cc24)oc2cc4cc(N(c5ccc(C(C)C)cc5)c5ccc([Si](C)(C)C)cc5)ccc4cc23)cc1 ; CC(C)c1ccc(N(c2ccc(F)cc2)c2ccc3cc4c(cc3c2)oc2cc3c(cc24)oc2cc4cc(N(c5ccc(F)cc5)c5ccc(C(C)C)cc5)ccc4cc23)cc1 ; CC(C)c1ccc(N(c2ccc(C#N)cc2)c2ccc3cc4c(cc3c2)oc2cc3c(cc24)oc2cc4cc(N(c5ccc(C#N)cc5)c5ccc(C(C)C)cc5)ccc4cc23)cc1]

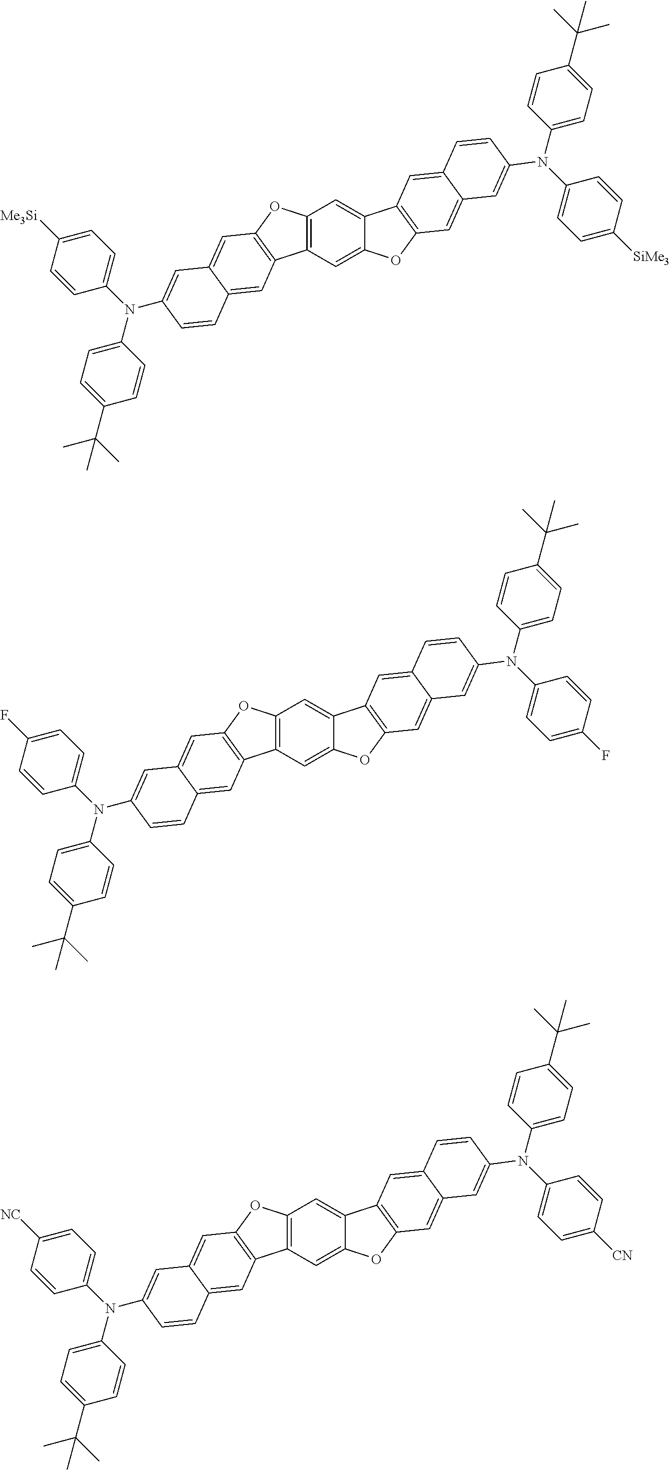 [SMILES: CC(C)(C)c1ccc(N(c2ccc([Si](C)(C)C)cc2)c2ccc3cc4c(cc3c2)oc2cc3c(cc24)oc2cc4cc(N(c5ccc(C(C)(C)C)cc5)c5ccc([Si](C)(C)C)cc5)ccc4cc23)cc1 ; CC(C)(C)c1ccc(N(c2ccc(F)cc2)c2ccc3cc4c(cc3c2)oc2cc3c(cc24)oc2cc4cc(N(c5ccc(F)cc5)c5ccc(C(C)(C)C)cc5)ccc4cc23)cc1 ; CC(C)(C)c1ccc(N(c2ccc(C#N)cc2)c2ccc3cc4c(cc3c2)oc2cc3c(cc24)oc2cc4cc(N(c5ccc(C#N)cc5)c5ccc(C(C)(C)C)cc5)ccc4cc23)cc1]

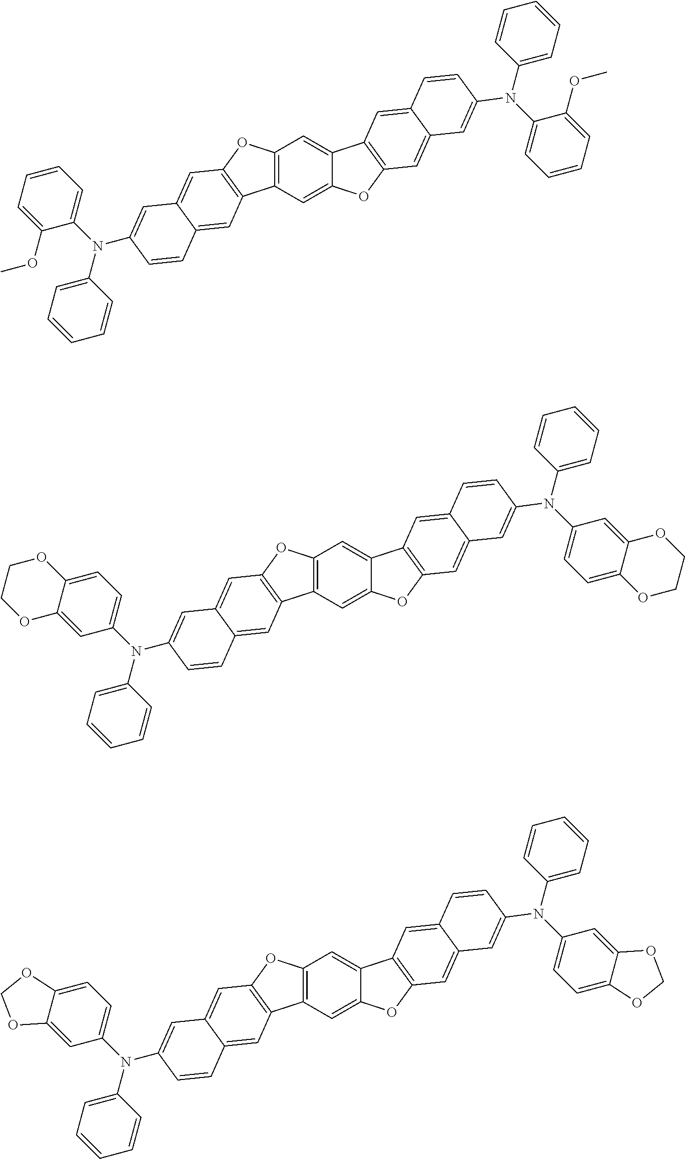

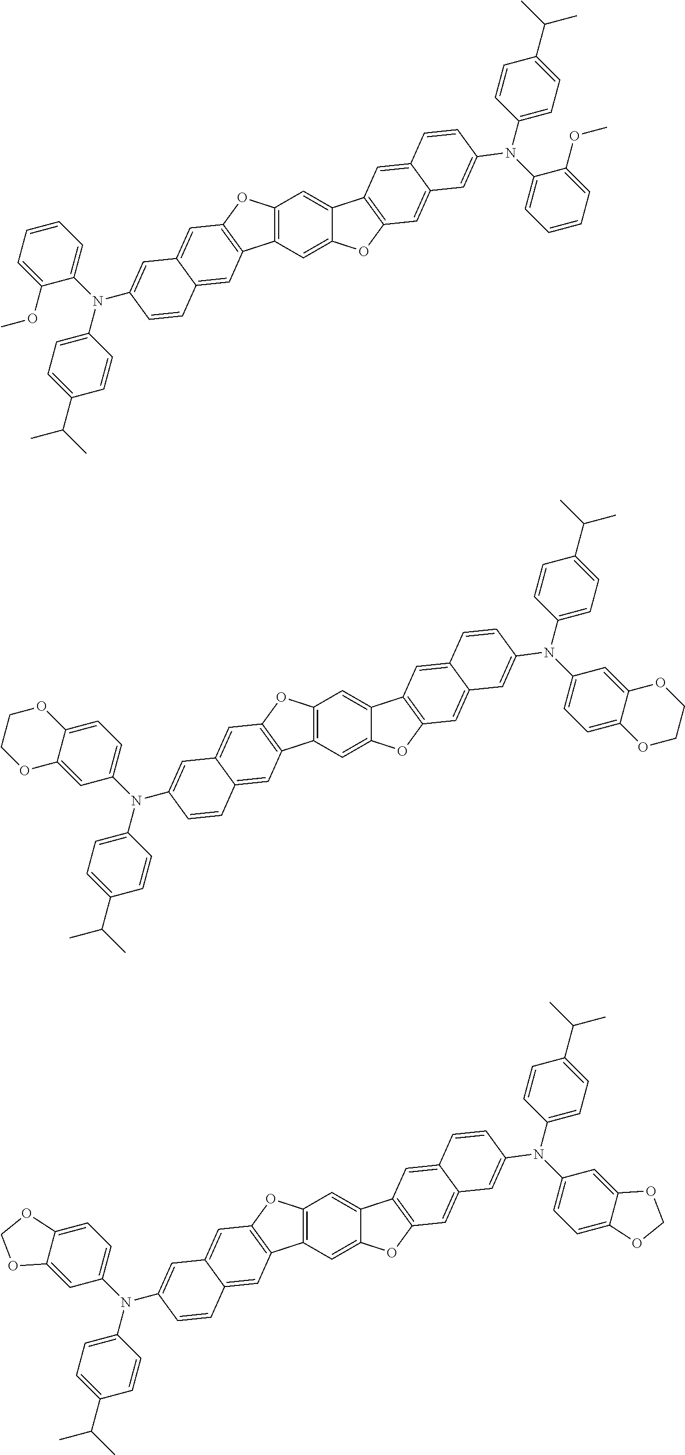

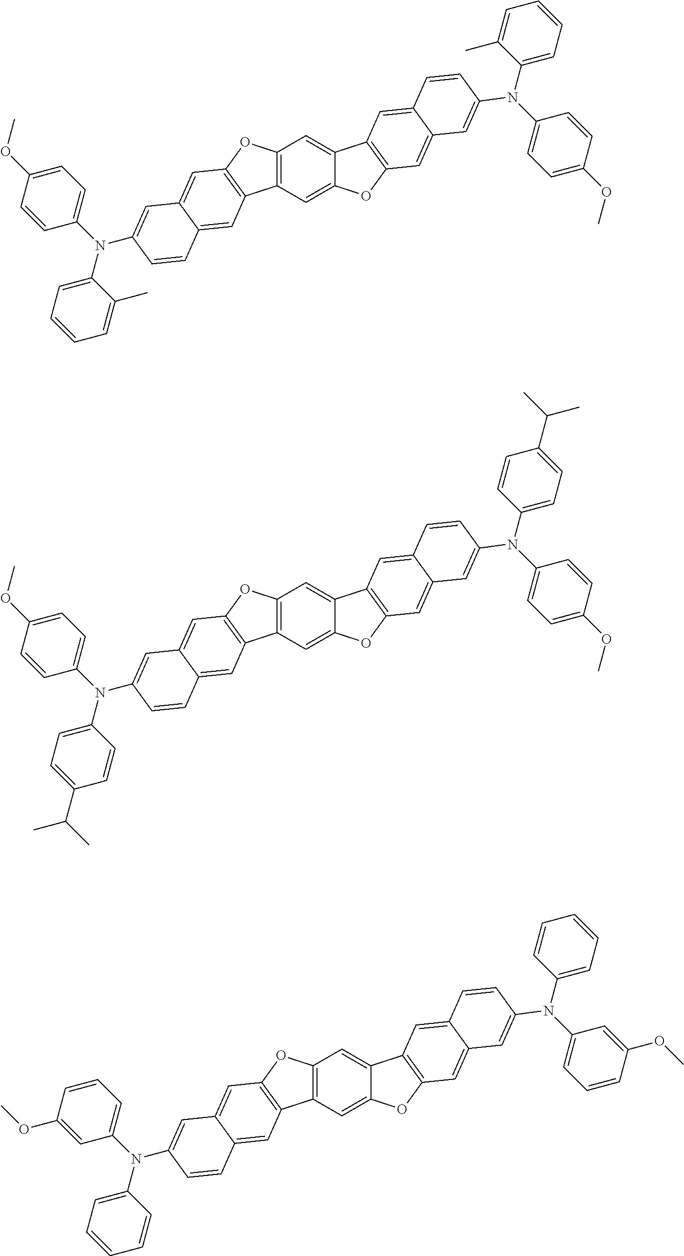

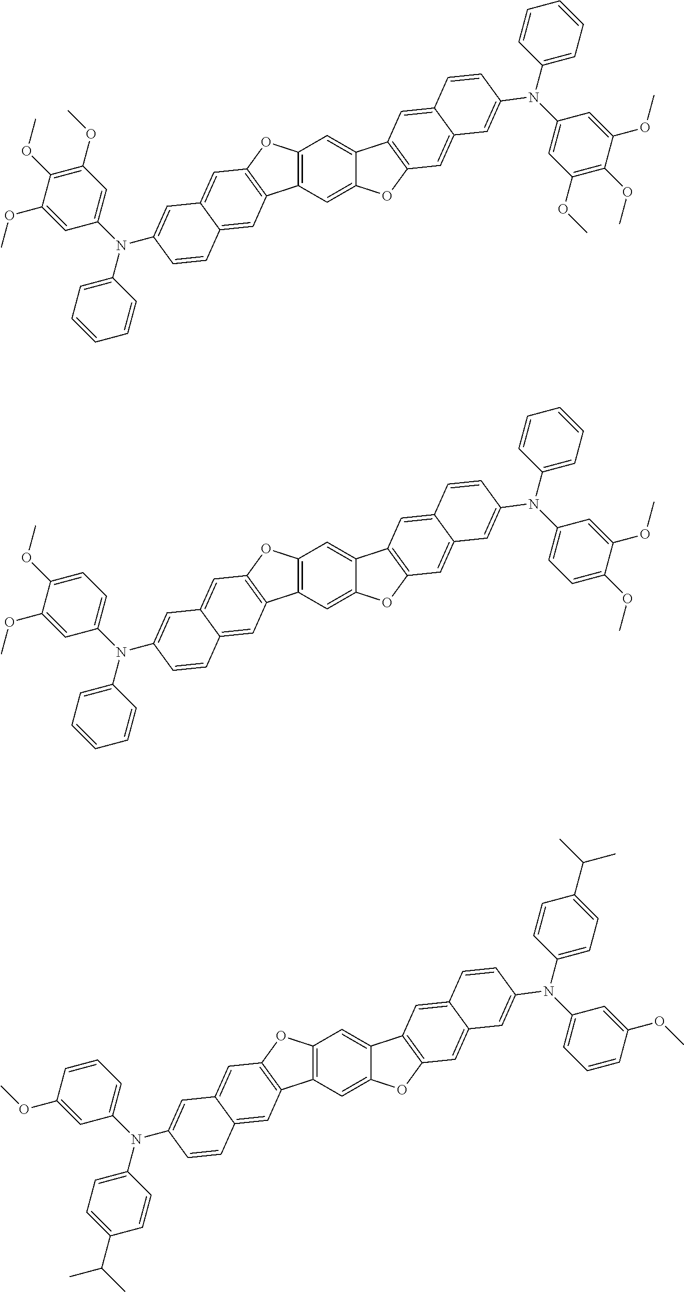

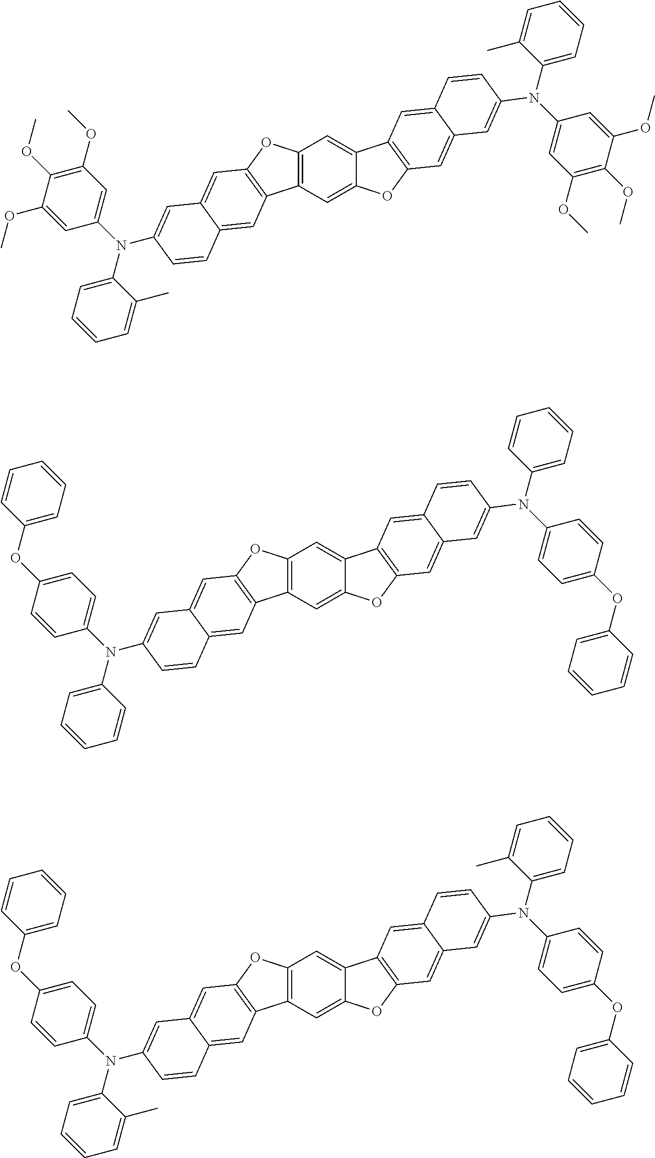

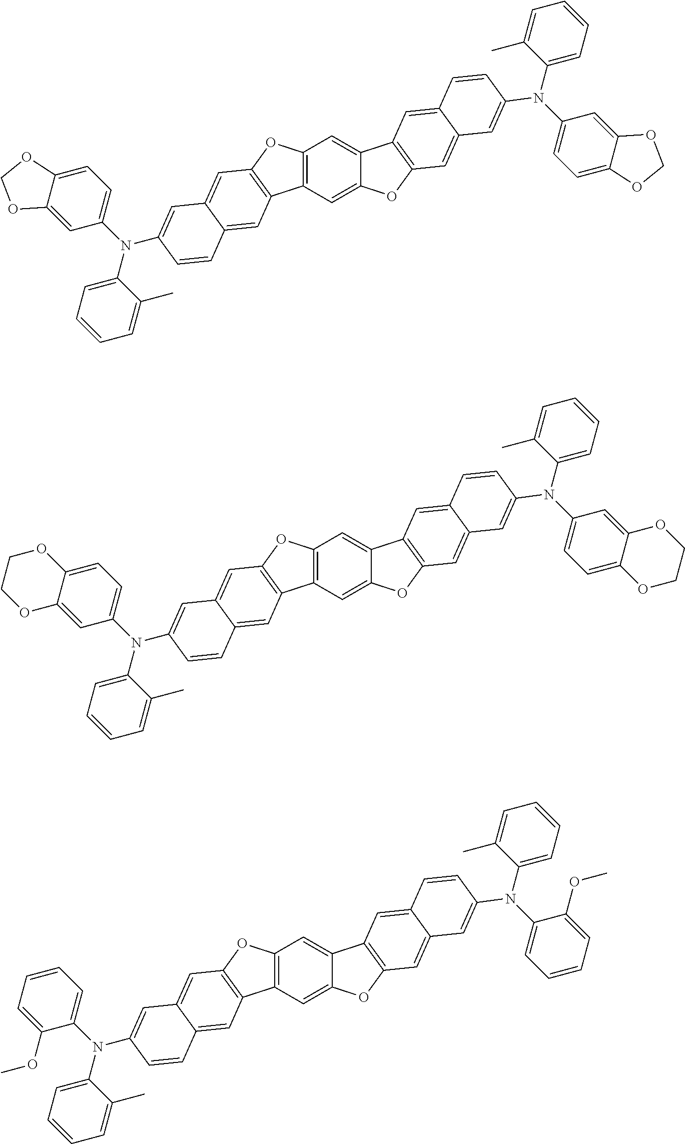

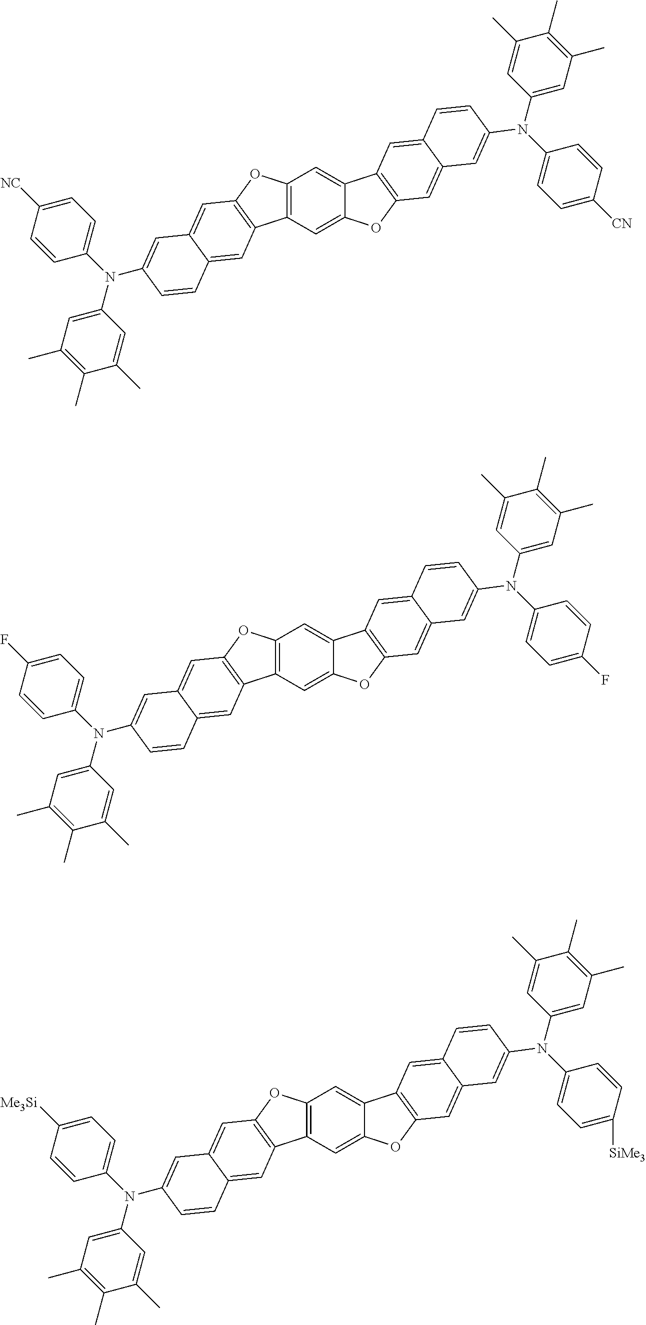 [SMILES: Cc1cc(N(c2ccc(C#N)cc2)c2ccc3cc4c(cc3c2)oc2cc3c(cc24)oc2cc4cc(N(c5ccc(C#N)cc5)c5cc(C)c(C)c(C)c5)ccc4cc23)cc(C)c1C ; Cc1cc(N(c2ccc(F)cc2)c2ccc3cc4c(cc3c2)oc2cc3c(cc24)oc2cc4cc(N(c5ccc(F)cc5)c5cc(C)c(C)c(C)c5)ccc4cc23)cc(C)c1C ; Cc1cc(N(c2ccc([Si](C)(C)C)cc2)c2ccc3cc4c(cc3c2)oc2cc3c(cc24)oc2cc4cc(N(c5ccc([Si](C)(C)C)cc5)c5cc(C)c(C)c(C)c5)ccc4cc23)cc(C)c1C]

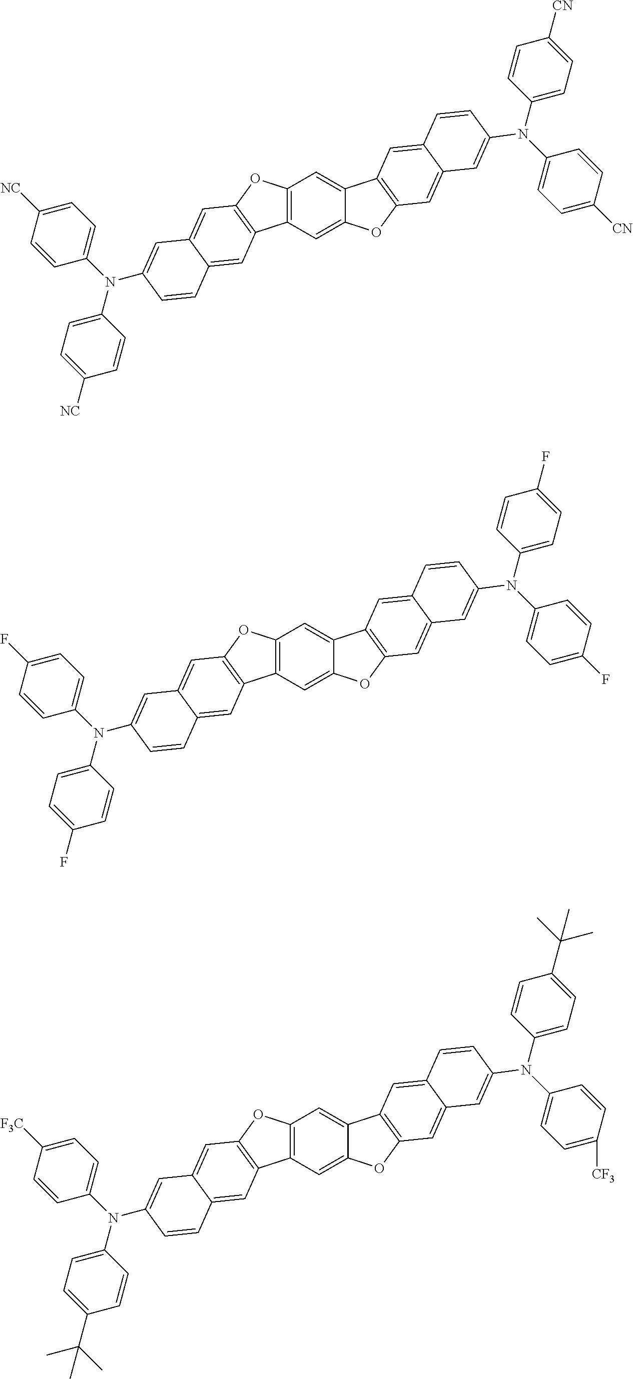 [SMILES: N#Cc1ccc(N(c2ccc(C#N)cc2)c2ccc3cc4c(cc3c2)oc2cc3c(cc24)oc2cc4cc(N(c5ccc(C#N)cc5)c5ccc(C#N)cc5)ccc4cc23)cc1 ; Fc1ccc(N(c2ccc(F)cc2)c2ccc3cc4c(cc3c2)oc2cc3c(cc24)oc2cc4cc(N(c5ccc(F)cc5)c5ccc(F)cc5)ccc4cc23)cc1 ; CC(C)(C)c1ccc(N(c2ccc(C(F)(F)F)cc2)c2ccc3cc4c(cc3c2)oc2cc3c(cc24)oc2cc4cc(N(c5ccc(C(C)(C)C)cc5)c5ccc(C(F)(F)F)cc5)ccc4cc23)cc1]

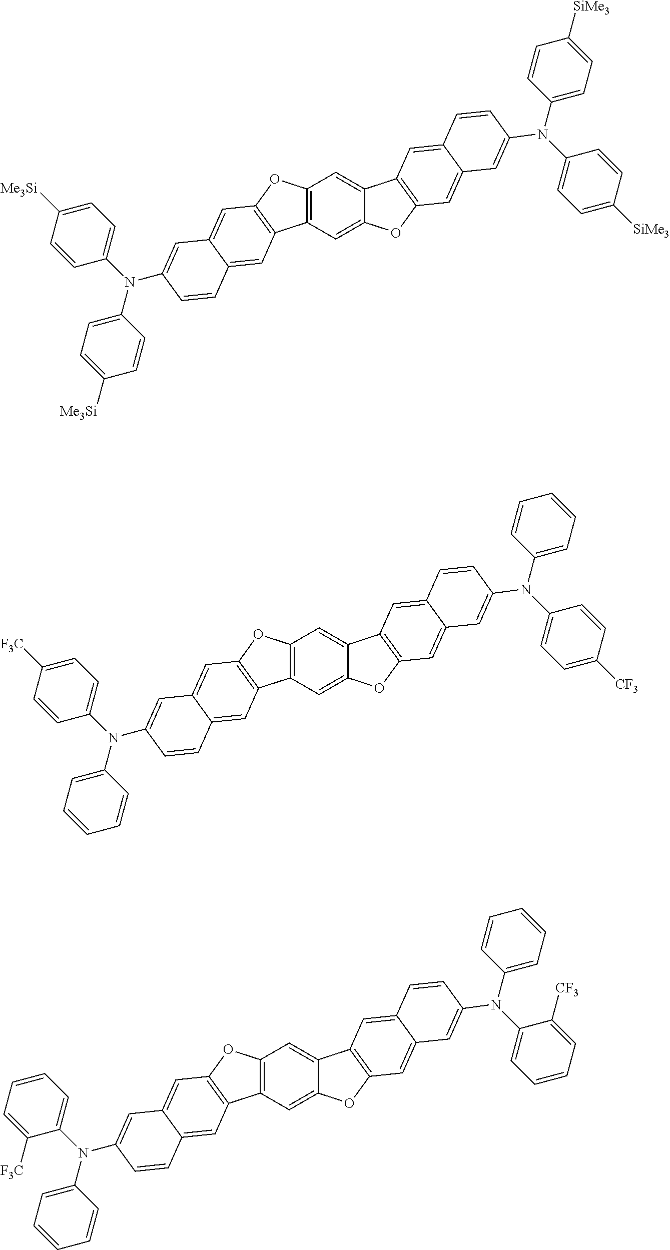 [SMILES: C[Si](C)(C)c1ccc(N(c2ccc([Si](C)(C)C)cc2)c2ccc3cc4c(cc3c2)oc2cc3c(cc24)oc2cc4cc(N(c5ccc([Si](C)(C)C)cc5)c5ccc([Si](C)(C)C)cc5)ccc4cc23)cc1 ; FC(F)(F)c1ccc(N(c2ccccc2)c2ccc3cc4c(cc3c2)oc2cc3c(cc24)oc2cc4cc(N(c5ccccc5)c5ccc(C(F)(F)F)cc5)ccc4cc23)cc1 ; FC(F)(F)c1ccccc1N(c1ccccc1)c1ccc2cc3c(cc2c1)oc1cc2c(cc13)oc1cc3cc(N(c4ccccc4)c4ccccc4C(F)(F)F)ccc3cc12]

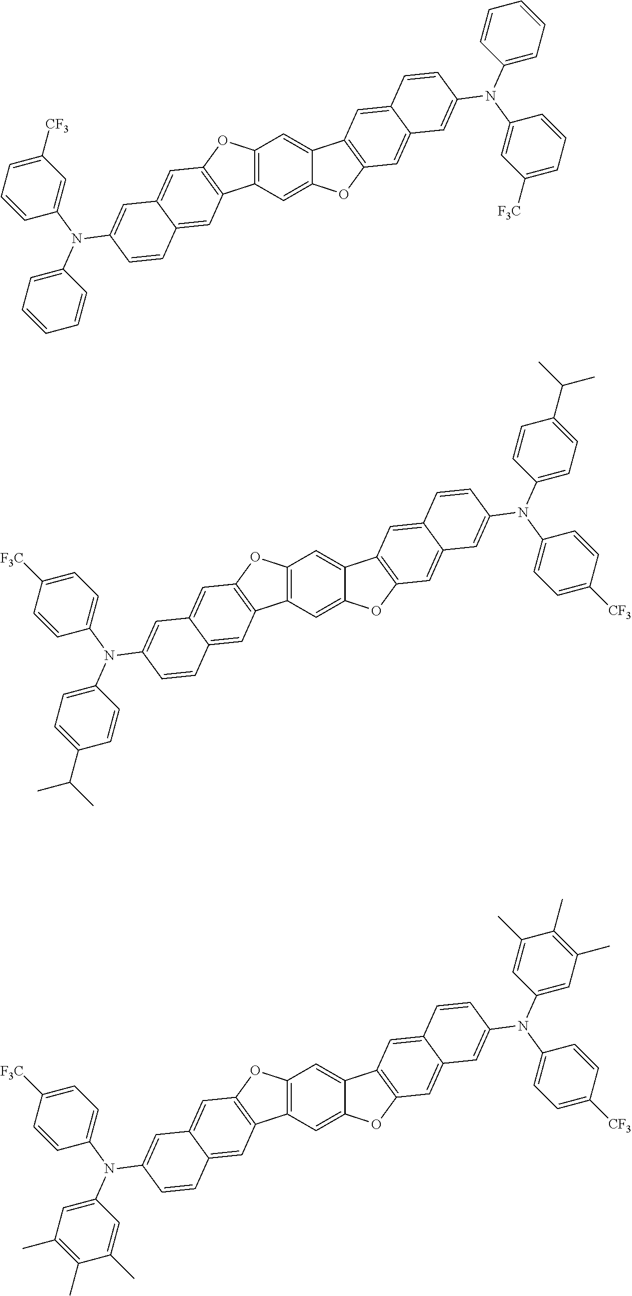 [SMILES: FC(F)(F)c1cccc(N(c2ccccc2)c2ccc3cc4c(cc3c2)oc2cc3c(cc24)oc2cc4cc(N(c5ccccc5)c5cccc(C(F)(F)F)c5)ccc4cc23)c1 ; CC(C)c1ccc(N(c2ccc(C(F)(F)F)cc2)c2ccc3cc4c(cc3c2)oc2cc3c(cc24)oc2cc4cc(N(c5ccc(C(C)C)cc5)c5ccc(C(F)(F)F)cc5)ccc4cc23)cc1 ; Cc1cc(N(c2ccc(C(F)(F)F)cc2)c2ccc3cc4c(cc3c2)oc2cc3c(cc24)oc2cc4cc(N(c5ccc(C(F)(F)F)cc5)c5cc(C)c(C)c(C)c5)ccc4cc23)cc(C)c1C]

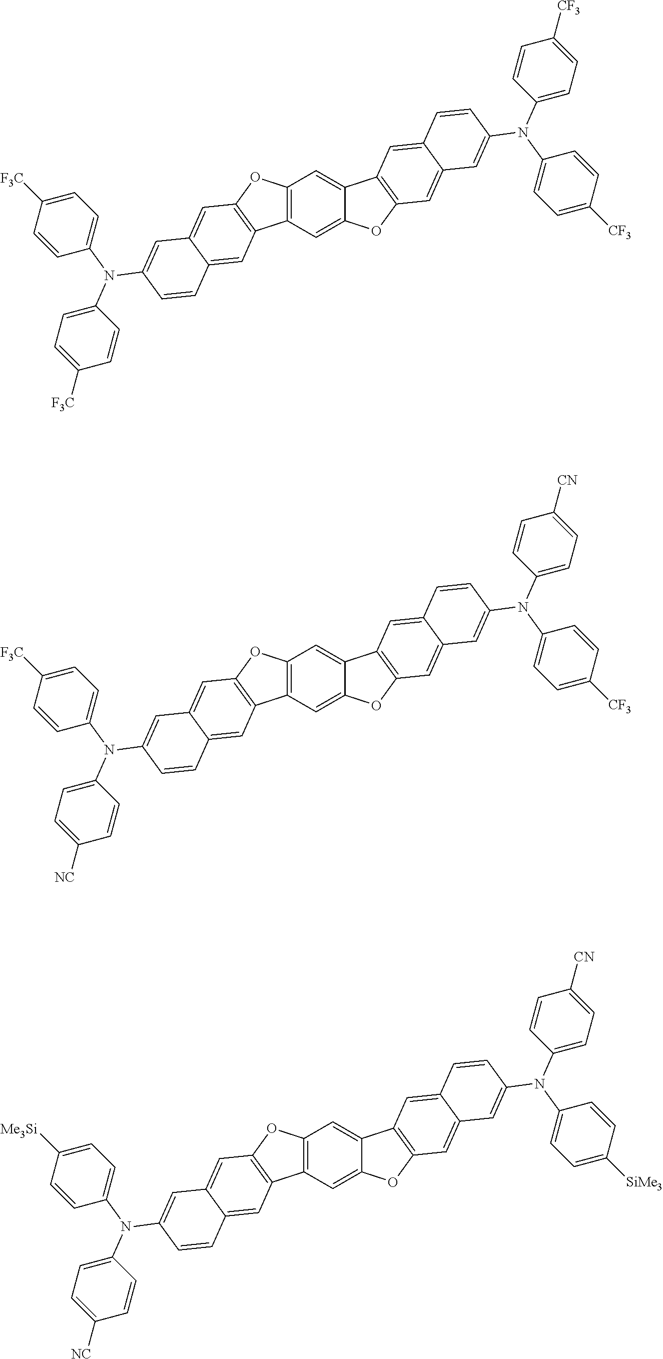 [SMILES: FC(F)(F)c1ccc(N(c2ccc(C(F)(F)F)cc2)c2ccc3cc4c(cc3c2)oc2cc3c(cc24)oc2cc4cc(N(c5ccc(C(F)(F)F)cc5)c5ccc(C(F)(F)F)cc5)ccc4cc23)cc1 ; N#Cc1ccc(N(c2ccc(C(F)(F)F)cc2)c2ccc3cc4c(cc3c2)oc2cc3c(cc24)oc2cc4cc(N(c5ccc(C#N)cc5)c5ccc(C(F)(F)F)cc5)ccc4cc23)cc1 ; C[Si](C)(C)c1ccc(N(c2ccc(C#N)cc2)c2ccc3cc4c(cc3c2)oc2cc3c(cc24)oc2cc4cc(N(c5ccc(C#N)cc5)c5ccc([Si](C)(C)C)cc5)ccc4cc23)cc1]

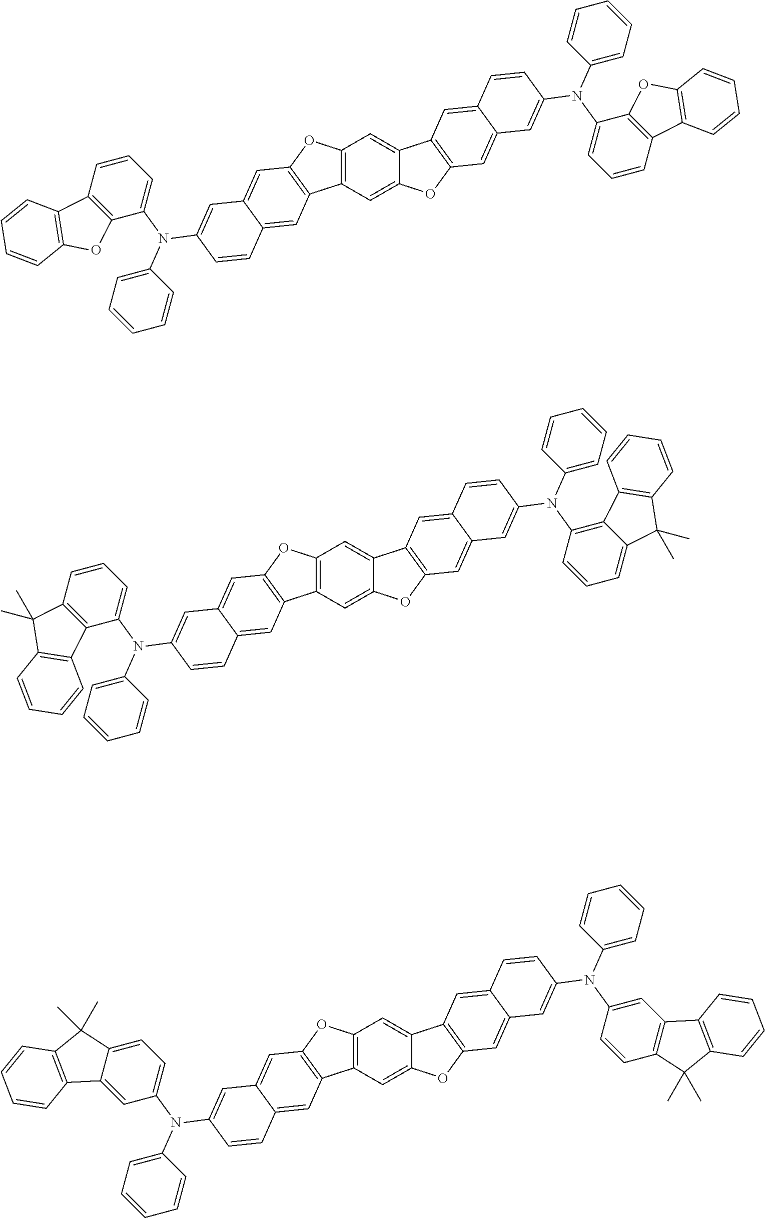

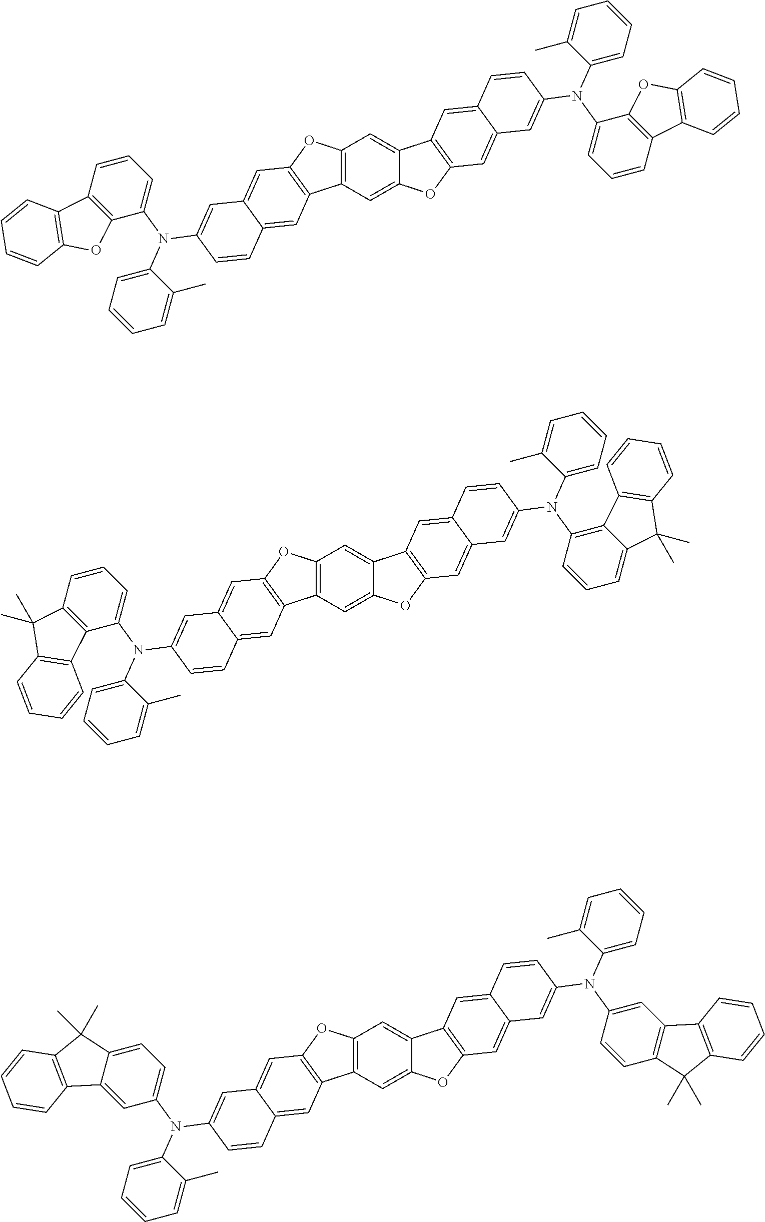

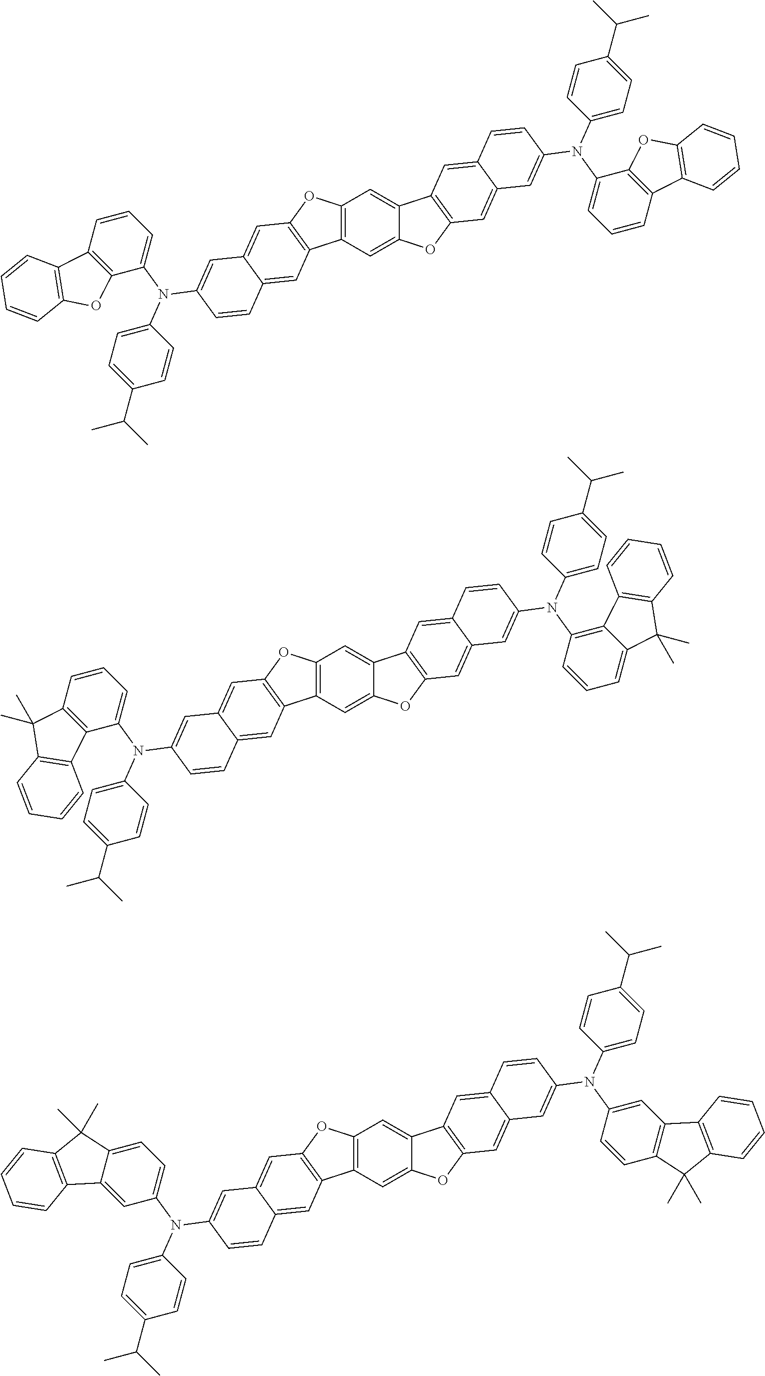

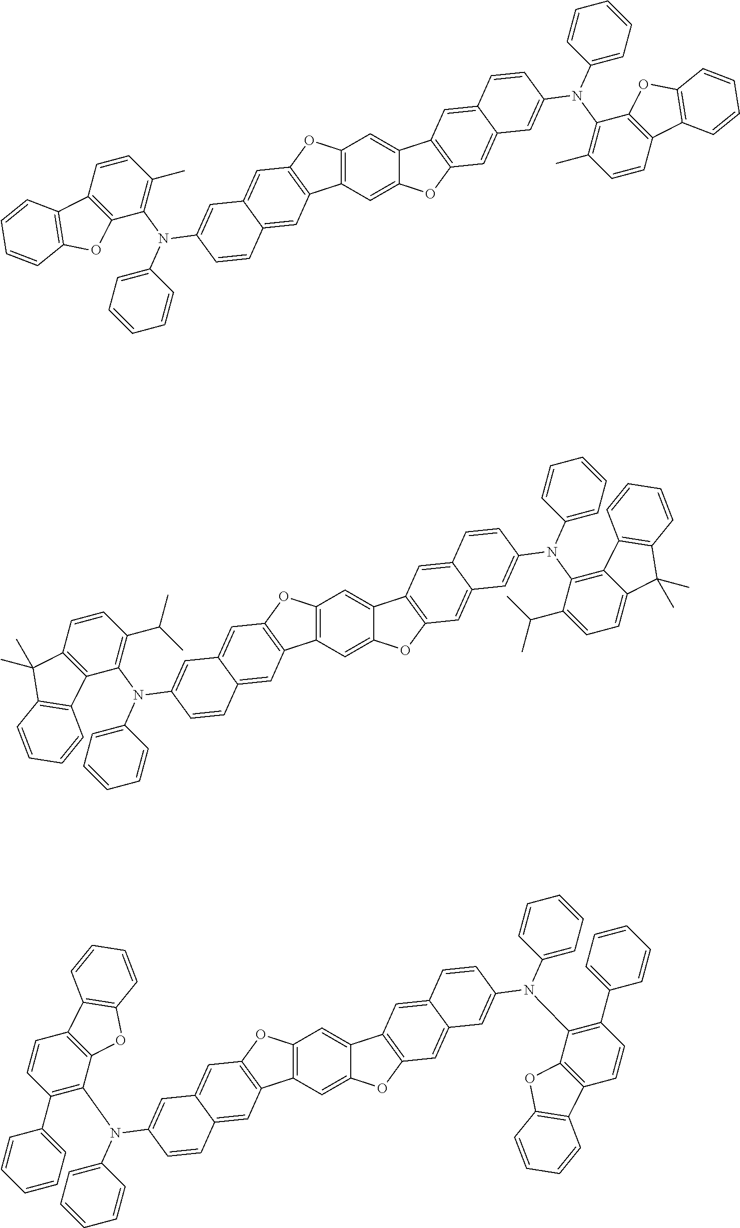

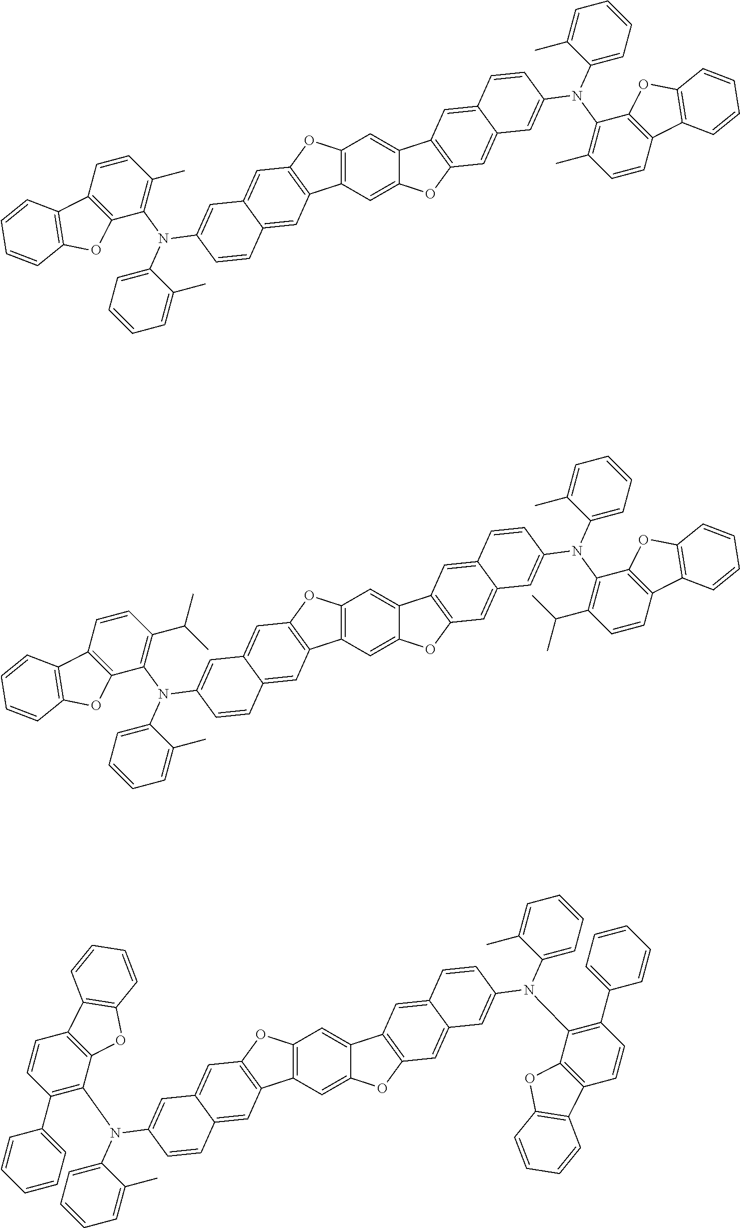 [SMILES: Cc1ccccc1N(c1ccc2cc3c(cc2c1)oc1cc2c(cc13)oc1cc3cc(N(c4ccccc4C)c4c(C)ccc5c4oc4ccccc45)ccc3cc12)c1c(C)ccc2c1oc1ccccc12 ; Cc1ccccc1N(c1ccc2cc3c(cc2c1)oc1cc2c(cc13)oc1cc3cc(N(c4ccccc4C)c4c(C(C)C)ccc5c4oc4ccccc45)ccc3cc12)c1c(C(C)C)ccc2c1oc1ccccc12 ; Cc1ccccc1N(c1ccc2cc3c(cc2c1)oc1cc2c(cc13)oc1cc3cc(N(c4ccccc4C)c4c(-c5ccccc5)ccc5c4oc4ccccc45)ccc3cc12)c1c(-c2ccccc2)ccc2c1oc1ccccc12]

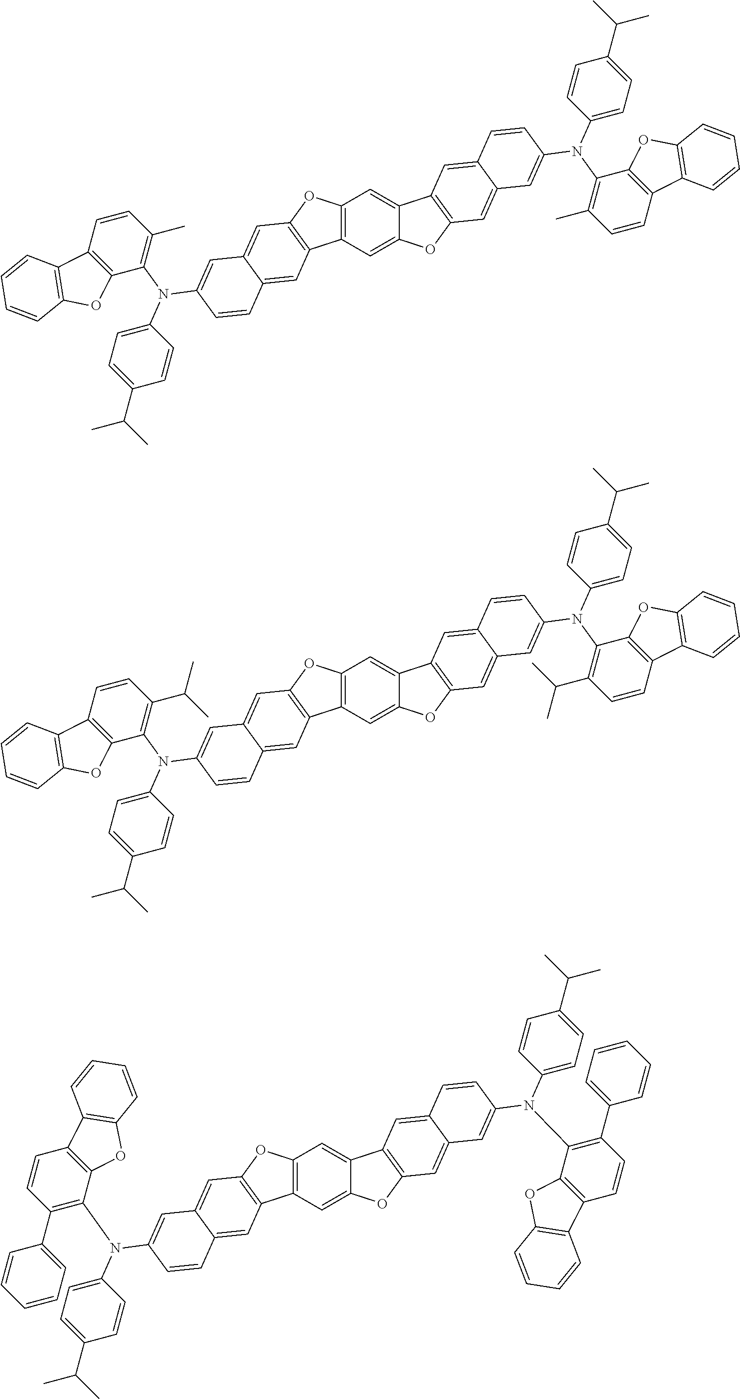 [SMILES: Cc1ccc2c(oc3ccccc32)c1N(c1ccc(C(C)C)cc1)c1ccc2cc3c(cc2c1)oc1cc2c(cc13)oc1cc3cc(N(c4ccc(C(C)C)cc4)c4c(C)ccc5c4oc4ccccc45)ccc3cc12 ; CC(C)c1ccc(N(c2ccc3cc4c(cc3c2)oc2cc3c(cc24)oc2cc4cc(N(c5ccc(C(C)C)cc5)c5c(C(C)C)ccc6c5oc5ccccc56)ccc4cc23)c2c(C(C)C)ccc3c2oc2ccccc23)cc1 ; CC(C)c1ccc(N(c2ccc3cc4c(cc3c2)oc2cc3c(cc24)oc2cc4cc(N(c5ccc(C(C)C)cc5)c5c(-c6ccccc6)ccc6c5oc5ccccc56)ccc4cc23)c2c(-c3ccccc3)ccc3c2oc2ccccc23)cc1]

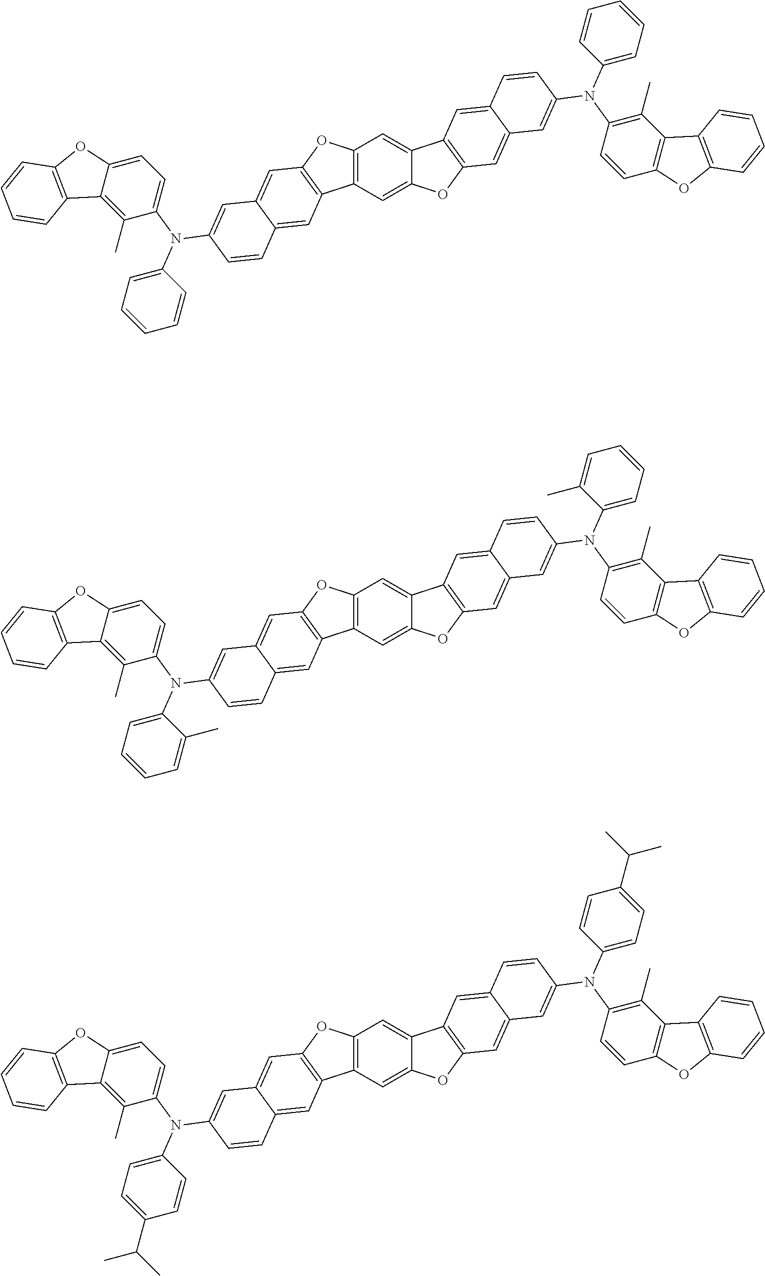 [SMILES: Cc1c(N(c2ccccc2)c2ccc3cc4c(cc3c2)oc2cc3c(cc24)oc2cc4cc(N(c5ccccc5)c5ccc6oc7ccccc7c6c5C)ccc4cc23)ccc2oc3ccccc3c12 ; Cc1ccccc1N(c1ccc2cc3c(cc2c1)oc1cc2c(cc13)oc1cc3cc(N(c4ccccc4C)c4ccc5oc6ccccc6c5c4C)ccc3cc12)c1ccc2oc3ccccc3c2c1C ; Cc1c(N(c2ccc(C(C)C)cc2)c2ccc3cc4c(cc3c2)oc2cc3c(cc24)oc2cc4cc(N(c5ccc(C(C)C)cc5)c5ccc6oc7ccccc7c6c5C)ccc4cc23)ccc2oc3ccccc3c12]

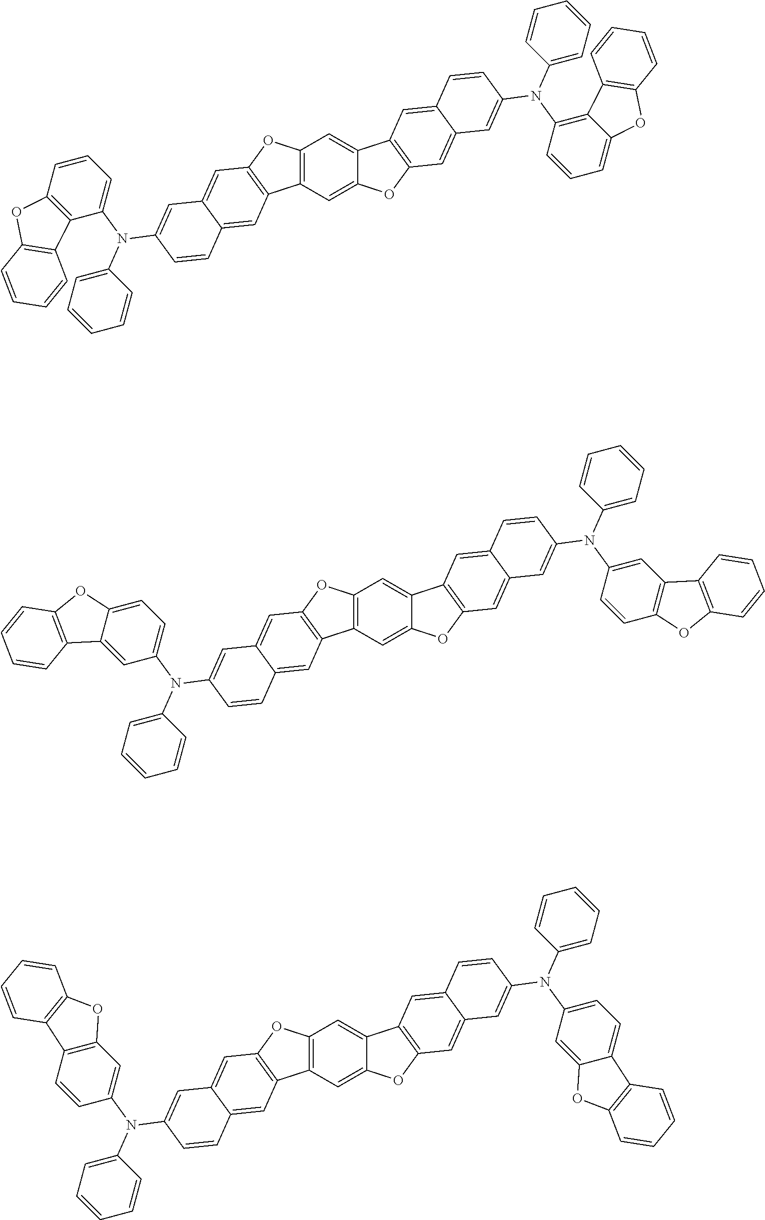 [SMILES: c1ccc(N(c2ccc3cc4c(cc3c2)oc2cc3c(cc24)oc2cc4cc(N(c5ccccc5)c5cccc6oc7ccccc7c56)ccc4cc23)c2cccc3oc4ccccc4c23)cc1 ; c1ccc(N(c2ccc3cc4c(cc3c2)oc2cc3c(cc24)oc2cc4cc(N(c5ccccc5)c5ccc6oc7ccccc7c6c5)ccc4cc23)c2ccc3oc4ccccc4c3c2)cc1 ; c1ccc(N(c2ccc3cc4c(cc3c2)oc2cc3c(cc24)oc2cc4cc(N(c5ccccc5)c5ccc6c(c5)oc5ccccc56)ccc4cc23)c2ccc3c(c2)oc2ccccc23)cc1]

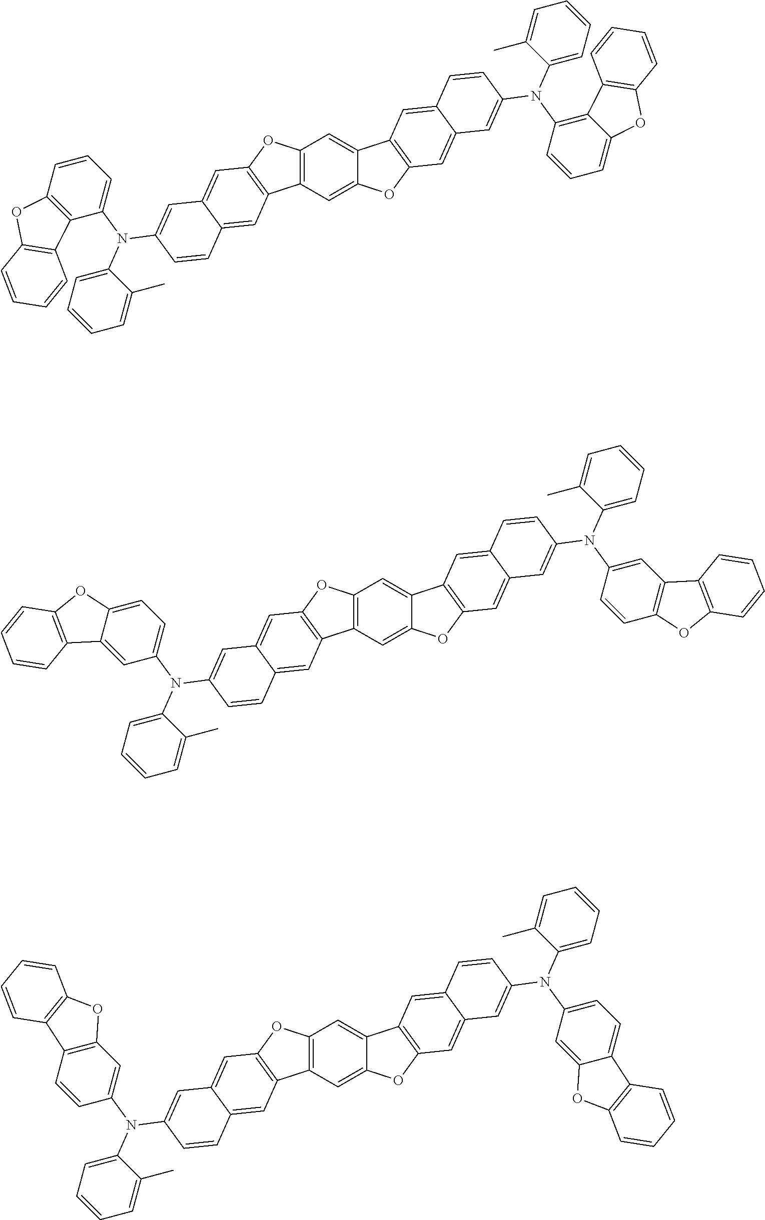 [SMILES: Cc1ccccc1N(c1ccc2cc3c(cc2c1)oc1cc2c(cc13)oc1cc3cc(N(c4ccccc4C)c4cccc5oc6ccccc6c45)ccc3cc12)c1cccc2oc3ccccc3c12 ; Cc1ccccc1N(c1ccc2cc3c(cc2c1)oc1cc2c(cc13)oc1cc3cc(N(c4ccc5oc6ccccc6c5c4)c4ccccc4C)ccc3cc12)c1ccc2oc3ccccc3c2c1 ; Cc1ccccc1N(c1ccc2cc3c(cc2c1)oc1cc2c(cc13)oc1cc3cc(N(c4ccc5c(c4)oc4ccccc45)c4ccccc4C)ccc3cc12)c1ccc2c(c1)oc1ccccc12]

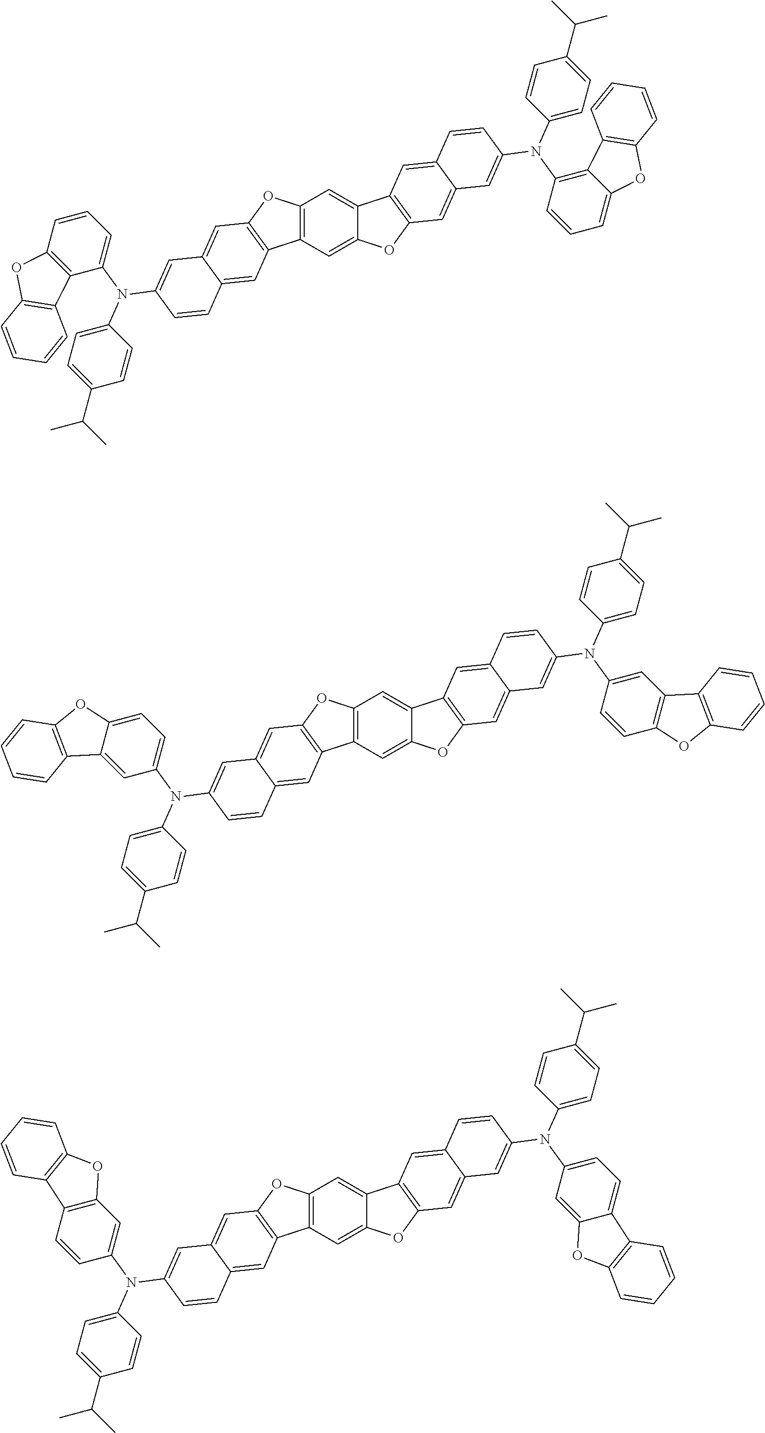 [SMILES: CC(C)c1ccc(N(c2ccc3cc4c(cc3c2)oc2cc3c(cc24)oc2cc4cc(N(c5ccc(C(C)C)cc5)c5cccc6oc7ccccc7c56)ccc4cc23)c2cccc3oc4ccccc4c23)cc1 ; CC(C)c1ccc(N(c2ccc3cc4c(cc3c2)oc2cc3c(cc24)oc2cc4cc(N(c5ccc(C(C)C)cc5)c5ccc6oc7ccccc7c6c5)ccc4cc23)c2ccc3oc4ccccc4c3c2)cc1 ; CC(C)c1ccc(N(c2ccc3cc4c(cc3c2)oc2cc3c(cc24)oc2cc4cc(N(c5ccc(C(C)C)cc5)c5ccc6c(c5)oc5ccccc56)ccc4cc23)c2ccc3c(c2)oc2ccccc23)cc1]

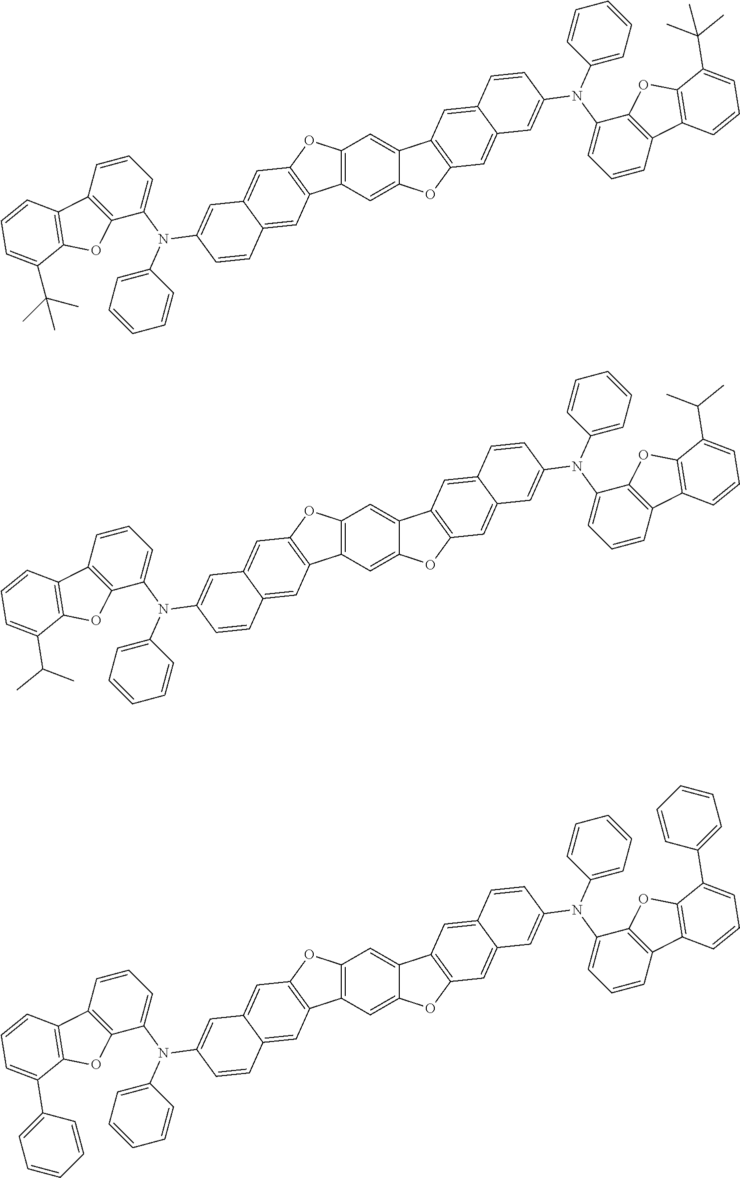 [SMILES: CC(C)(C)c1cccc2c1oc1c(N(c3ccccc3)c3ccc4cc5c(cc4c3)oc3cc4c(cc35)oc3cc5cc(N(c6ccccc6)c6cccc7c6oc6c(C(C)(C)C)cccc67)ccc5cc34)cccc12 ; CC(C)c1cccc2c1oc1c(N(c3ccccc3)c3ccc4cc5c(cc4c3)oc3cc4c(cc35)oc3cc5cc(N(c6ccccc6)c6cccc7c6oc6c(C(C)C)cccc67)ccc5cc34)cccc12 ; c1ccc(-c2cccc3c2oc2c(N(c4ccccc4)c4ccc5cc6c(cc5c4)oc4cc5c(cc46)oc4cc6cc(N(c7ccccc7)c7cccc8c7oc7c(-c9ccccc9)cccc78)ccc6cc45)cccc23)cc1]

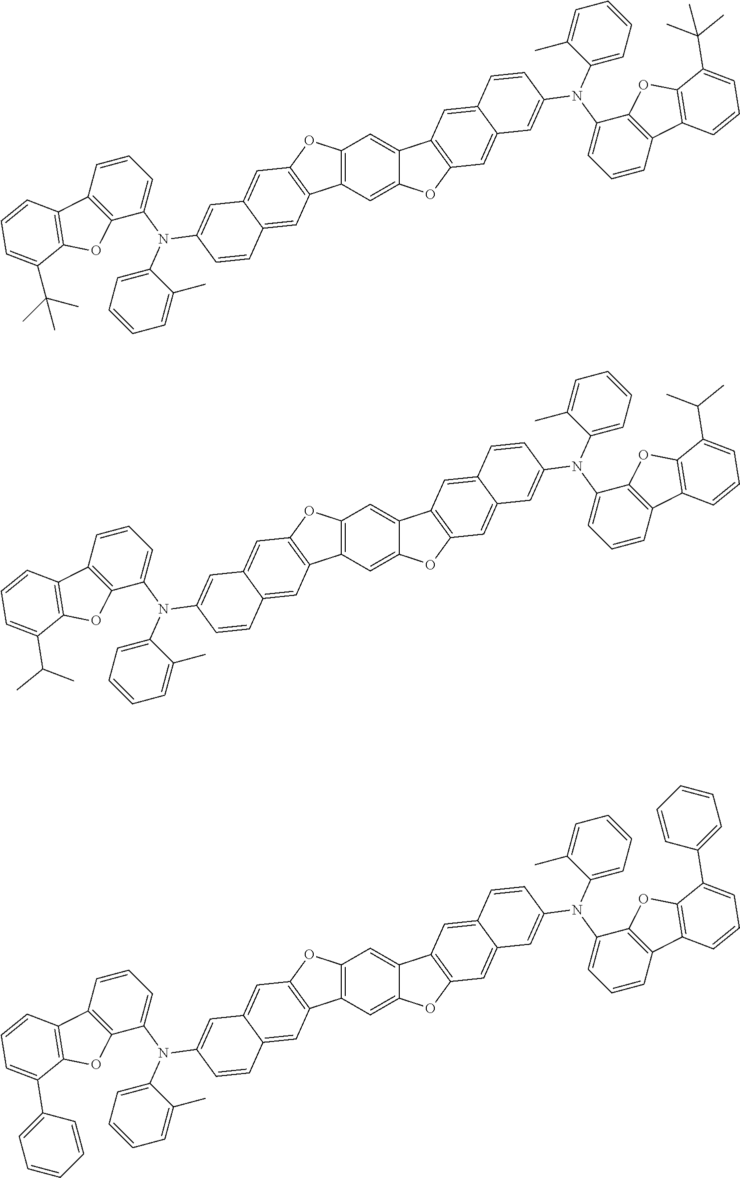 [SMILES: Cc1ccccc1N(c1ccc2cc3c(cc2c1)oc1cc2c(cc13)oc1cc3cc(N(c4ccccc4C)c4cccc5c4oc4c(C(C)(C)C)cccc45)ccc3cc12)c1cccc2c1oc1c(C(C)(C)C)cccc12 ; Cc1ccccc1N(c1ccc2cc3c(cc2c1)oc1cc2c(cc13)oc1cc3cc(N(c4ccccc4C)c4cccc5c4oc4c(C(C)C)cccc45)ccc3cc12)c1cccc2c1oc1c(C(C)C)cccc12 ; Cc1ccccc1N(c1ccc2cc3c(cc2c1)oc1cc2c(cc13)oc1cc3cc(N(c4ccccc4C)c4cccc5c4oc4c(-c6ccccc6)cccc45)ccc3cc12)c1cccc2c1oc1c(-c3ccccc3)cccc12]

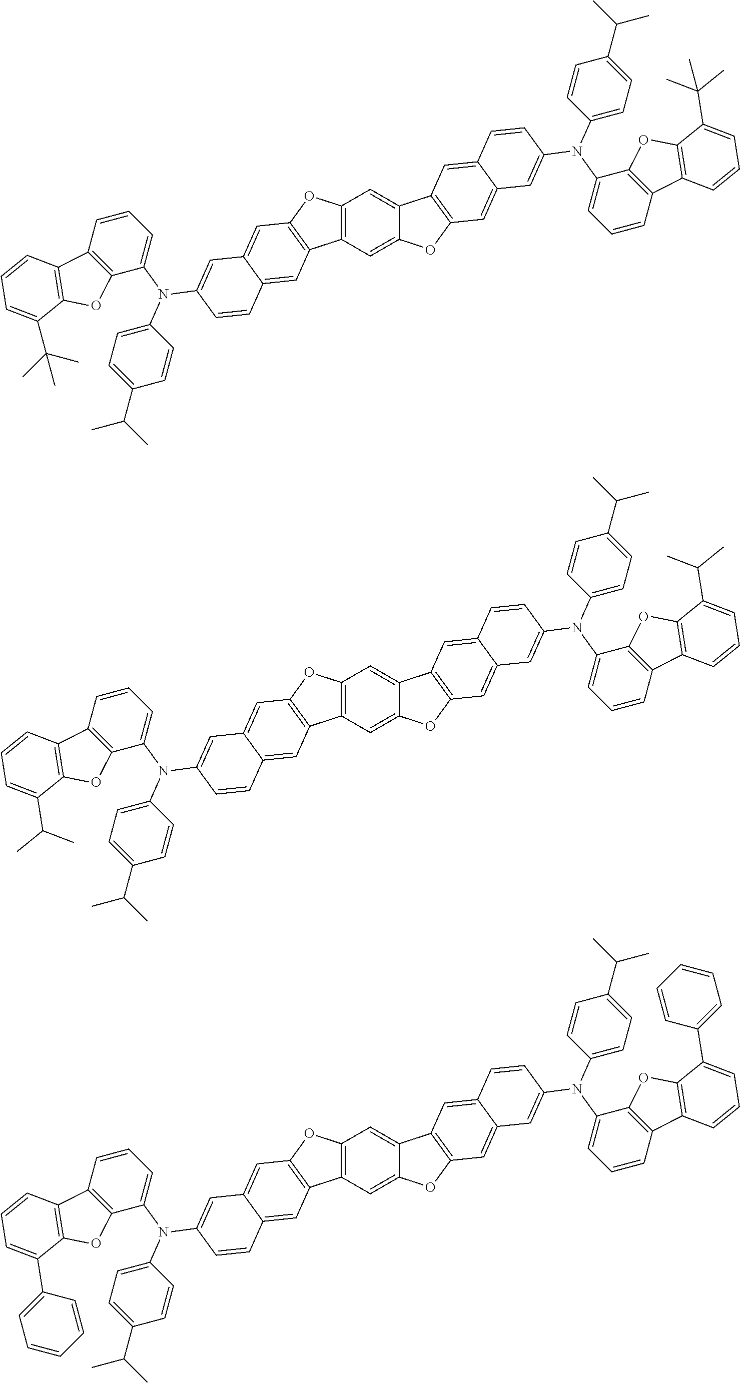 [SMILES: CC(C)c1ccc(N(c2ccc3cc4c(cc3c2)oc2cc3c(cc24)oc2cc4cc(N(c5ccc(C(C)C)cc5)c5cccc6c5oc5c(C(C)(C)C)cccc56)ccc4cc23)c2cccc3c2oc2c(C(C)(C)C)cccc23)cc1 ; CC(C)c1ccc(N(c2ccc3cc4c(cc3c2)oc2cc3c(cc24)oc2cc4cc(N(c5ccc(C(C)C)cc5)c5cccc6c5oc5c(C(C)C)cccc56)ccc4cc23)c2cccc3c2oc2c(C(C)C)cccc23)cc1 ; CC(C)c1ccc(N(c2ccc3cc4c(cc3c2)oc2cc3c(cc24)oc2cc4cc(N(c5ccc(C(C)C)cc5)c5cccc6c5oc5c(-c7ccccc7)cccc56)ccc4cc23)c2cccc3c2oc2c(-c4ccccc4)cccc23)cc1]

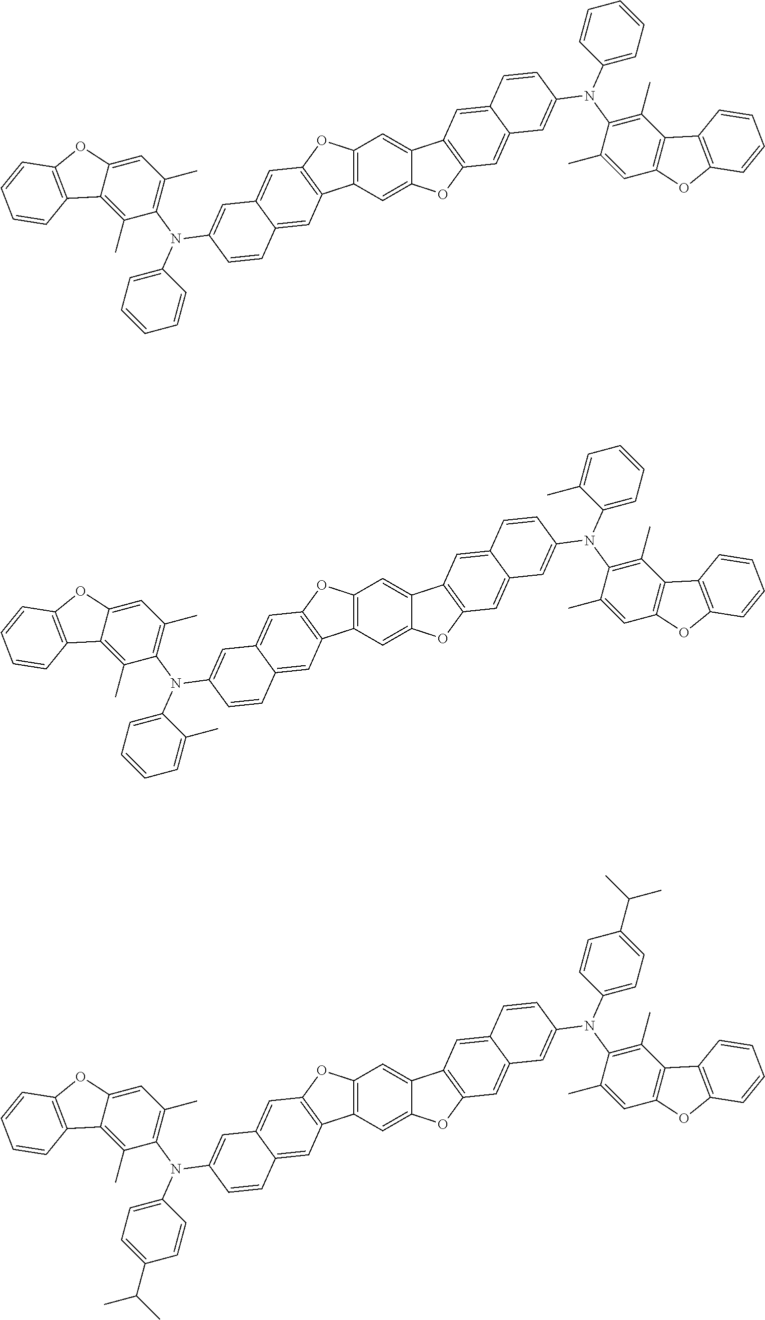 [SMILES: Cc1cc2oc3ccccc3c2c(C)c1N(c1ccccc1)c1ccc2cc3c(cc2c1)oc1cc2c(cc13)oc1cc3cc(N(c4ccccc4)c4c(C)cc5oc6ccccc6c5c4C)ccc3cc12 ; Cc1ccccc1N(c1ccc2cc3c(cc2c1)oc1cc2c(cc13)oc1cc3cc(N(c4ccccc4C)c4c(C)cc5oc6ccccc6c5c4C)ccc3cc12)c1c(C)cc2oc3ccccc3c2c1C ; Cc1cc2oc3ccccc3c2c(C)c1N(c1ccc(C(C)C)cc1)c1ccc2cc3c(cc2c1)oc1cc2c(cc13)oc1cc3cc(N(c4ccc(C(C)C)cc4)c4c(C)cc5oc6ccccc6c5c4C)ccc3cc12]

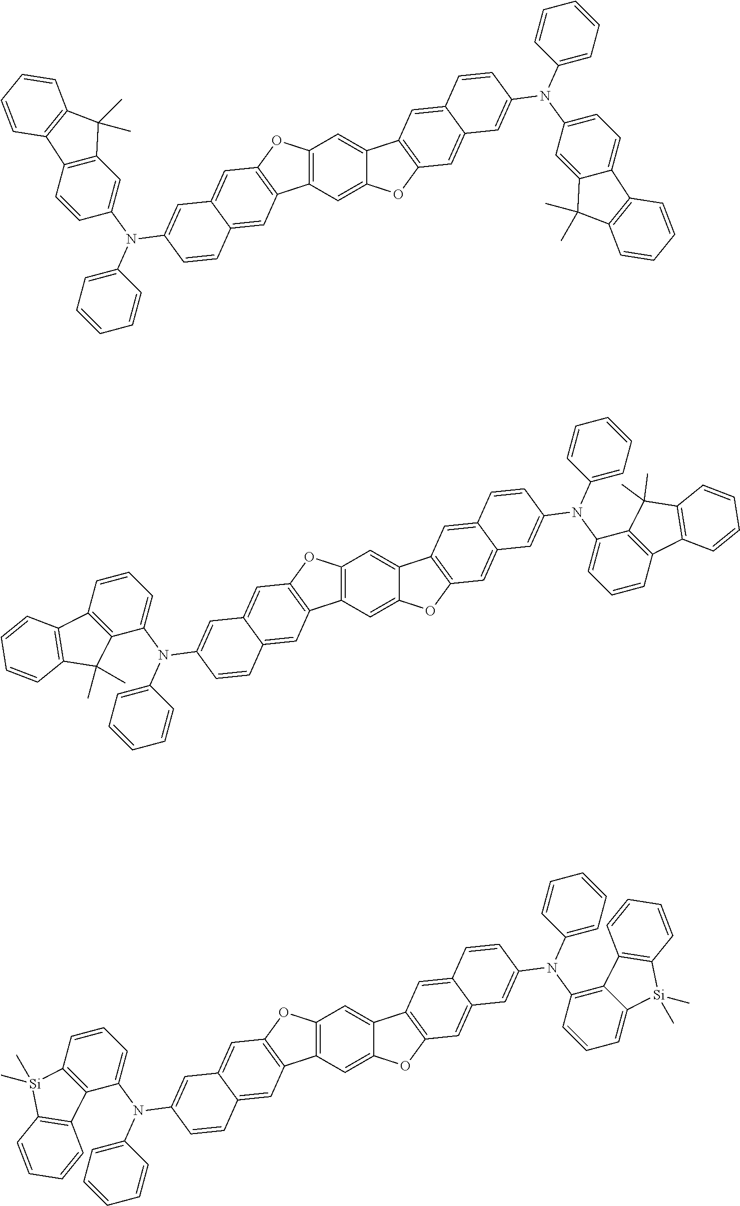

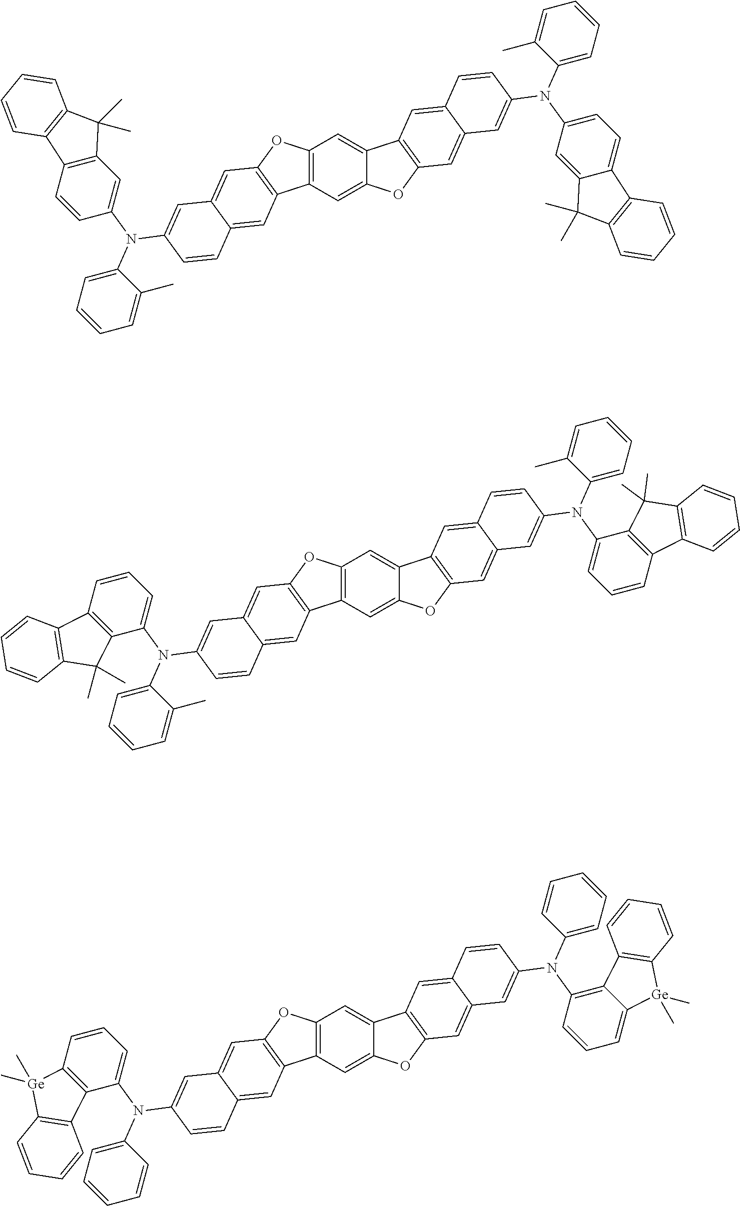

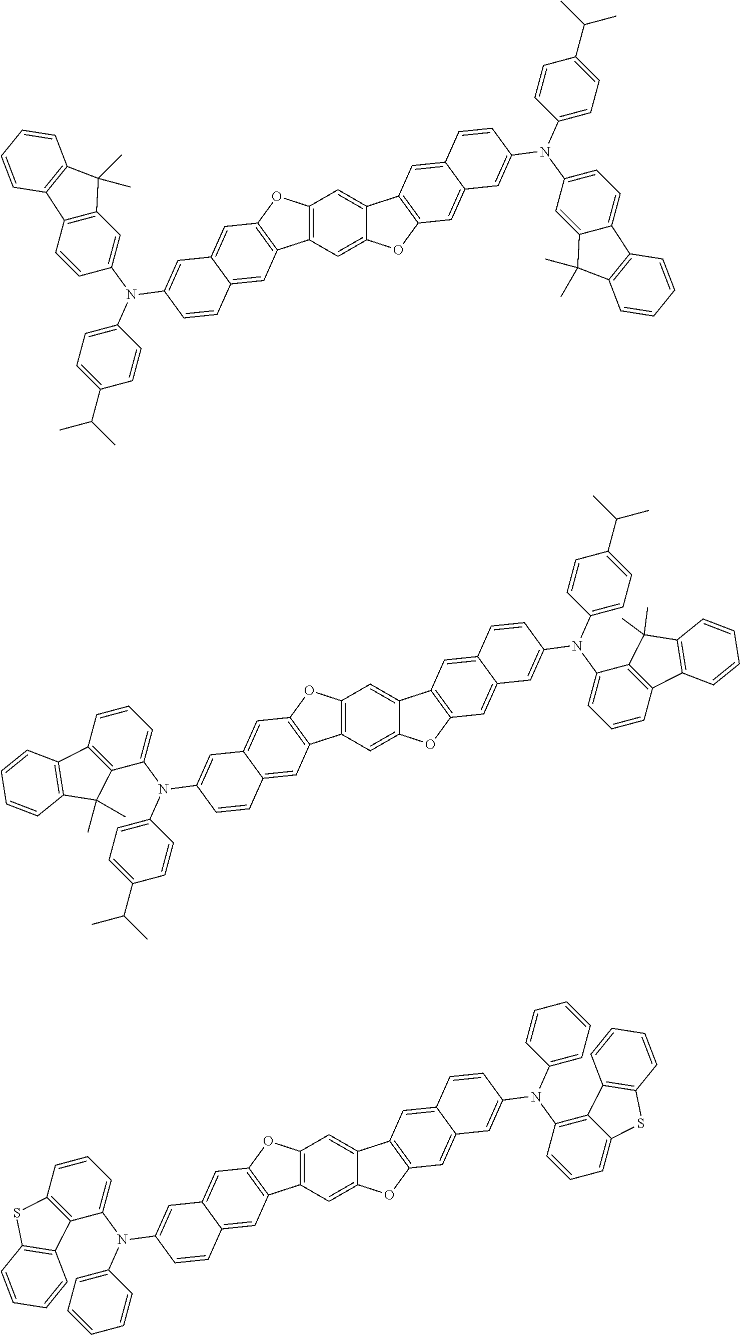 [SMILES: CC(C)c1ccc(N(c2ccc3c(c2)C(C)(C)c2ccccc2-3)c2ccc3cc4c(cc3c2)oc2cc3c(cc24)oc2cc4cc(N(c5ccc(C(C)C)cc5)c5ccc6c(c5)C(C)(C)c5ccccc5-6)ccc4cc23)cc1 ; CC(C)c1ccc(N(c2ccc3cc4c(cc3c2)oc2cc3c(cc24)oc2cc4cc(N(c5ccc(C(C)C)cc5)c5cccc6c5C(C)(C)c5ccccc5-6)ccc4cc23)c2cccc3c2C(C)(C)c2ccccc2-3)cc1 ; c1ccc(N(c2ccc3cc4c(cc3c2)oc2cc3c(cc24)oc2cc4cc(N(c5ccccc5)c5cccc6sc7ccccc7c56)ccc4cc23)c2cccc3sc4ccccc4c23)cc1]

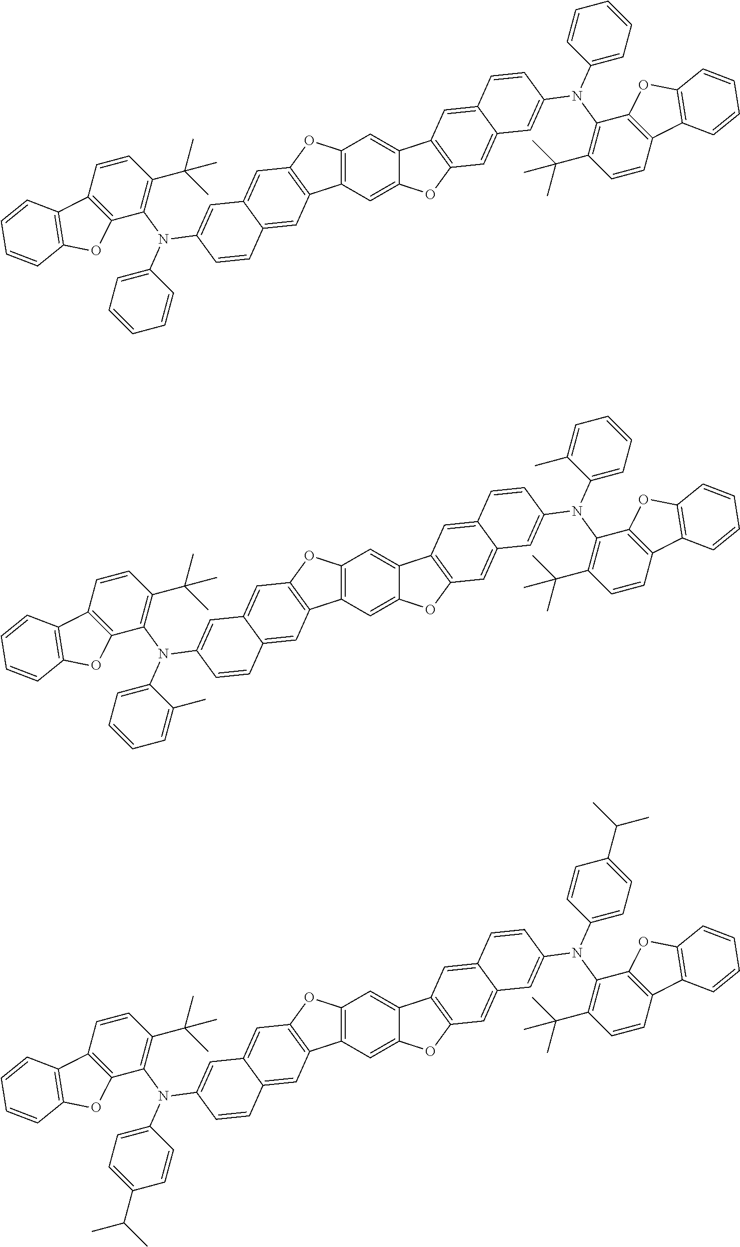 [SMILES: CC(C)(C)c1ccc2c(oc3ccccc32)c1N(c1ccccc1)c1ccc2cc3c(cc2c1)oc1cc2c(cc13)oc1cc3cc(N(c4ccccc4)c4c(C(C)(C)C)ccc5c4oc4ccccc45)ccc3cc12 ; Cc1ccccc1N(c1ccc2cc3c(cc2c1)oc1cc2c(cc13)oc1cc3cc(N(c4ccccc4C)c4c(C(C)(C)C)ccc5c4oc4ccccc45)ccc3cc12)c1c(C(C)(C)C)ccc2c1oc1ccccc12 ; CC(C)c1ccc(N(c2ccc3cc4c(cc3c2)oc2cc3c(cc24)oc2cc4cc(N(c5ccc(C(C)C)cc5)c5c(C(C)(C)C)ccc6c5oc5ccccc56)ccc4cc23)c2c(C(C)(C)C)ccc3c2oc2ccccc23)cc1]

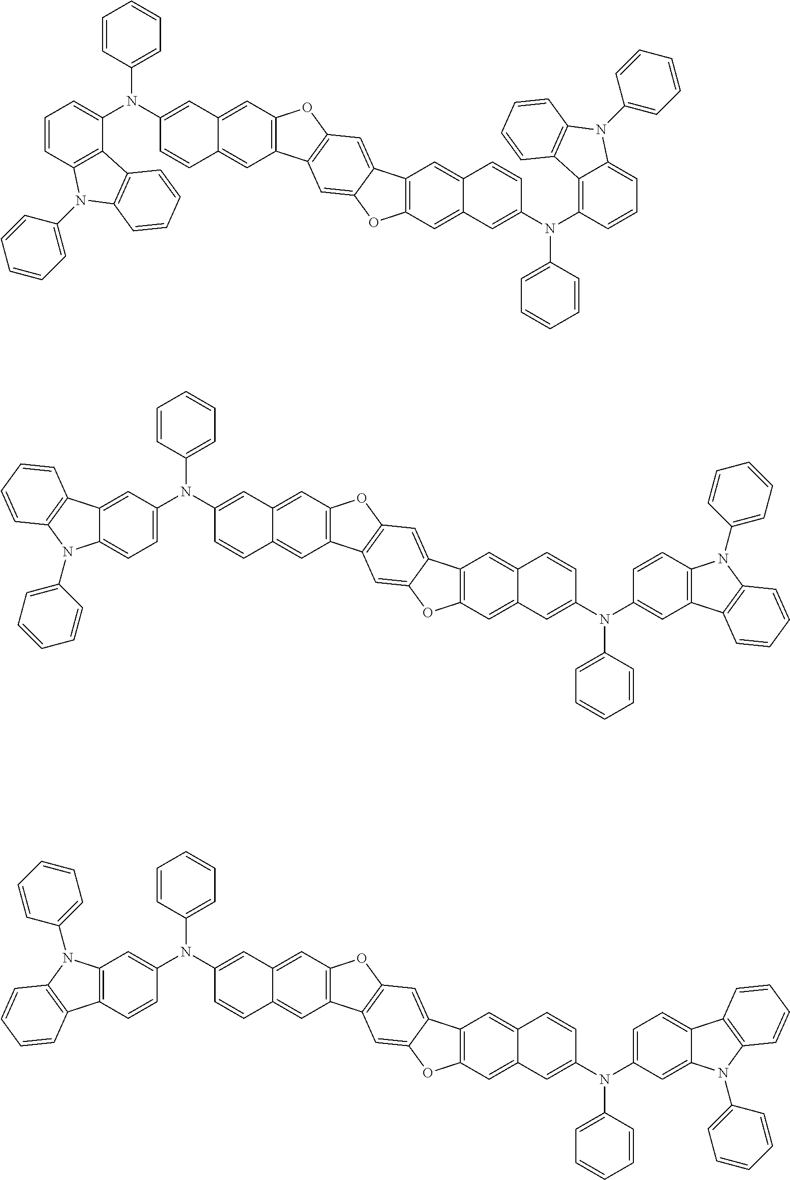 [SMILES: c1ccc(N(c2ccc3cc4c(cc3c2)oc2cc3c(cc24)oc2cc4cc(N(c5ccccc5)c5cccc6c5c5ccccc5n6-c5ccccc5)ccc4cc23)c2cccc3c2c2ccccc2n3-c2ccccc2)cc1 ; c1ccc(N(c2ccc3cc4c(cc3c2)oc2cc3c(cc24)oc2cc4cc(N(c5ccccc5)c5ccc6c(c5)c5ccccc5n6-c5ccccc5)ccc4cc23)c2ccc3c(c2)c2ccccc2n3-c2ccccc2)cc1 ; c1ccc(N(c2ccc3cc4c(cc3c2)oc2cc3c(cc24)oc2cc4cc(N(c5ccccc5)c5ccc6c7ccccc7n(-c7ccccc7)c6c5)ccc4cc23)c2ccc3c4ccccc4n(-c4ccccc4)c3c2)cc1]

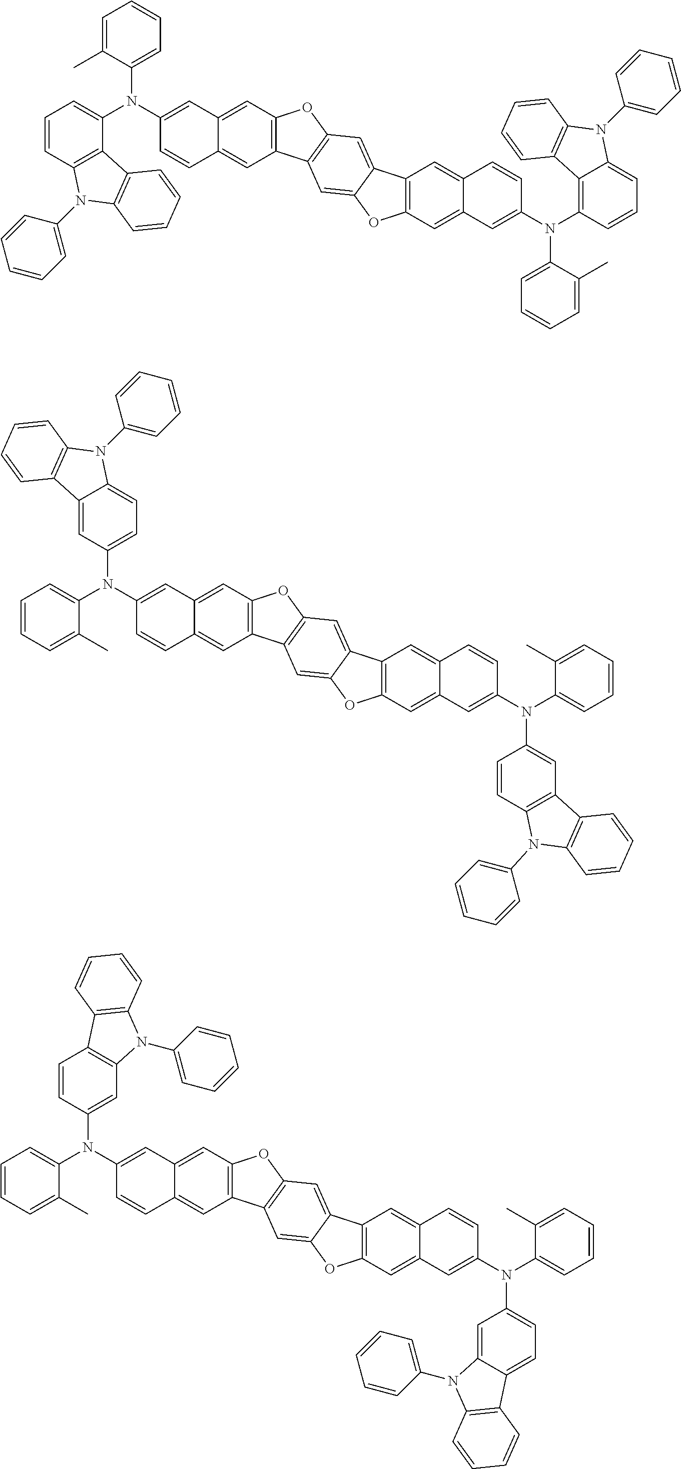 [SMILES: Cc1ccccc1N(c1ccc2cc3c(cc2c1)oc1cc2c(cc13)oc1cc3cc(N(c4ccccc4C)c4cccc5c4c4ccccc4n5-c4ccccc4)ccc3cc12)c1cccc2c1c1ccccc1n2-c1ccccc1 ; Cc1ccccc1N(c1ccc2cc3c(cc2c1)oc1cc2c(cc13)oc1cc3cc(N(c4ccc5c(c4)c4ccccc4n5-c4ccccc4)c4ccccc4C)ccc3cc12)c1ccc2c(c1)c1ccccc1n2-c1ccccc1 ; Cc1ccccc1N(c1ccc2cc3c(cc2c1)oc1cc2c(cc13)oc1cc3cc(N(c4ccc5c6ccccc6n(-c6ccccc6)c5c4)c4ccccc4C)ccc3cc12)c1ccc2c3ccccc3n(-c3ccccc3)c2c1]

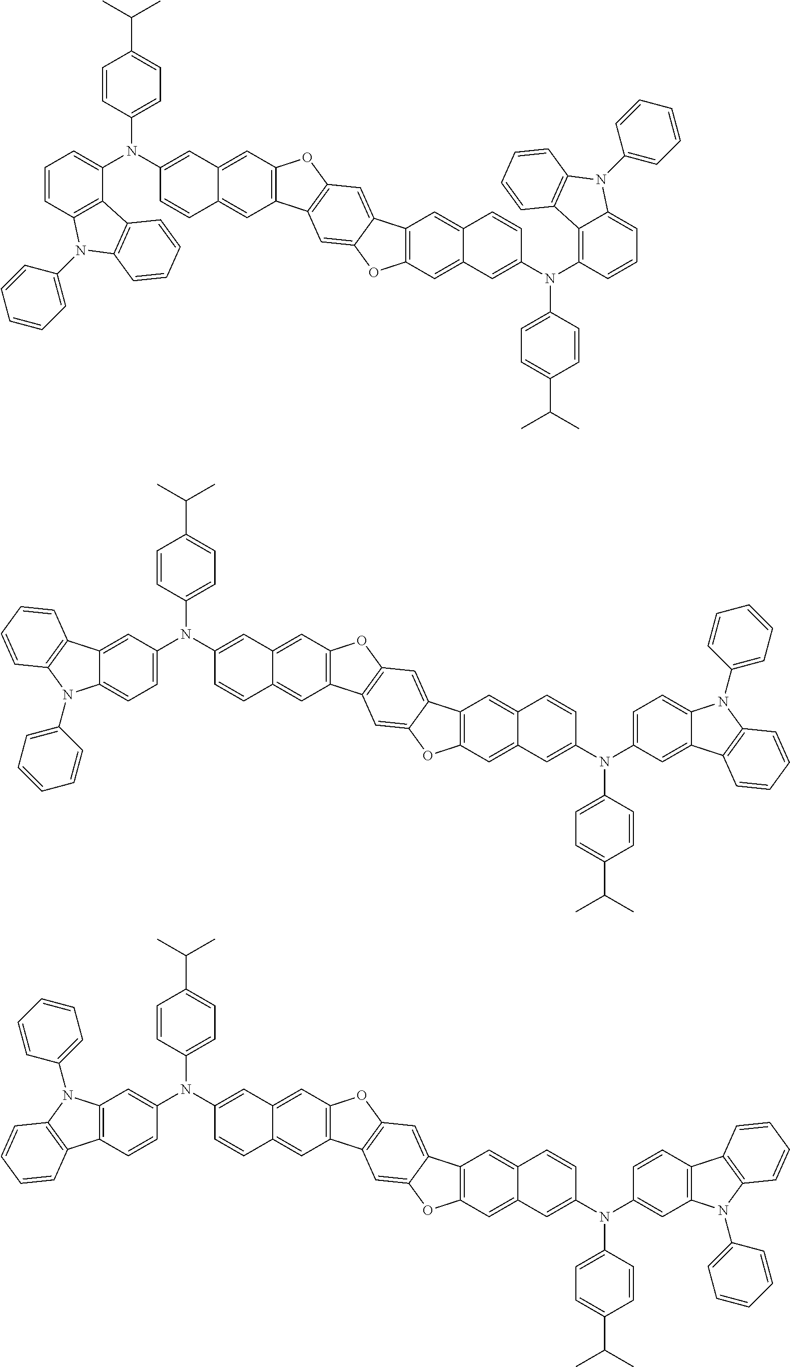 [SMILES: CC(C)c1ccc(N(c2ccc3cc4c(cc3c2)oc2cc3c(cc24)oc2cc4cc(N(c5ccc(C(C)C)cc5)c5cccc6c5c5ccccc5n6-c5ccccc5)ccc4cc23)c2cccc3c2c2ccccc2n3-c2ccccc2)cc1 ; CC(C)c1ccc(N(c2ccc3cc4c(cc3c2)oc2cc3c(cc24)oc2cc4cc(N(c5ccc(C(C)C)cc5)c5ccc6c(c5)c5ccccc5n6-c5ccccc5)ccc4cc23)c2ccc3c(c2)c2ccccc2n3-c2ccccc2)cc1 ; CC(C)c1ccc(N(c2ccc3cc4c(cc3c2)oc2cc3c(cc24)oc2cc4cc(N(c5ccc(C(C)C)cc5)c5ccc6c7ccccc7n(-c7ccccc7)c6c5)ccc4cc23)c2ccc3c4ccccc4n(-c4ccccc4)c3c2)cc1]

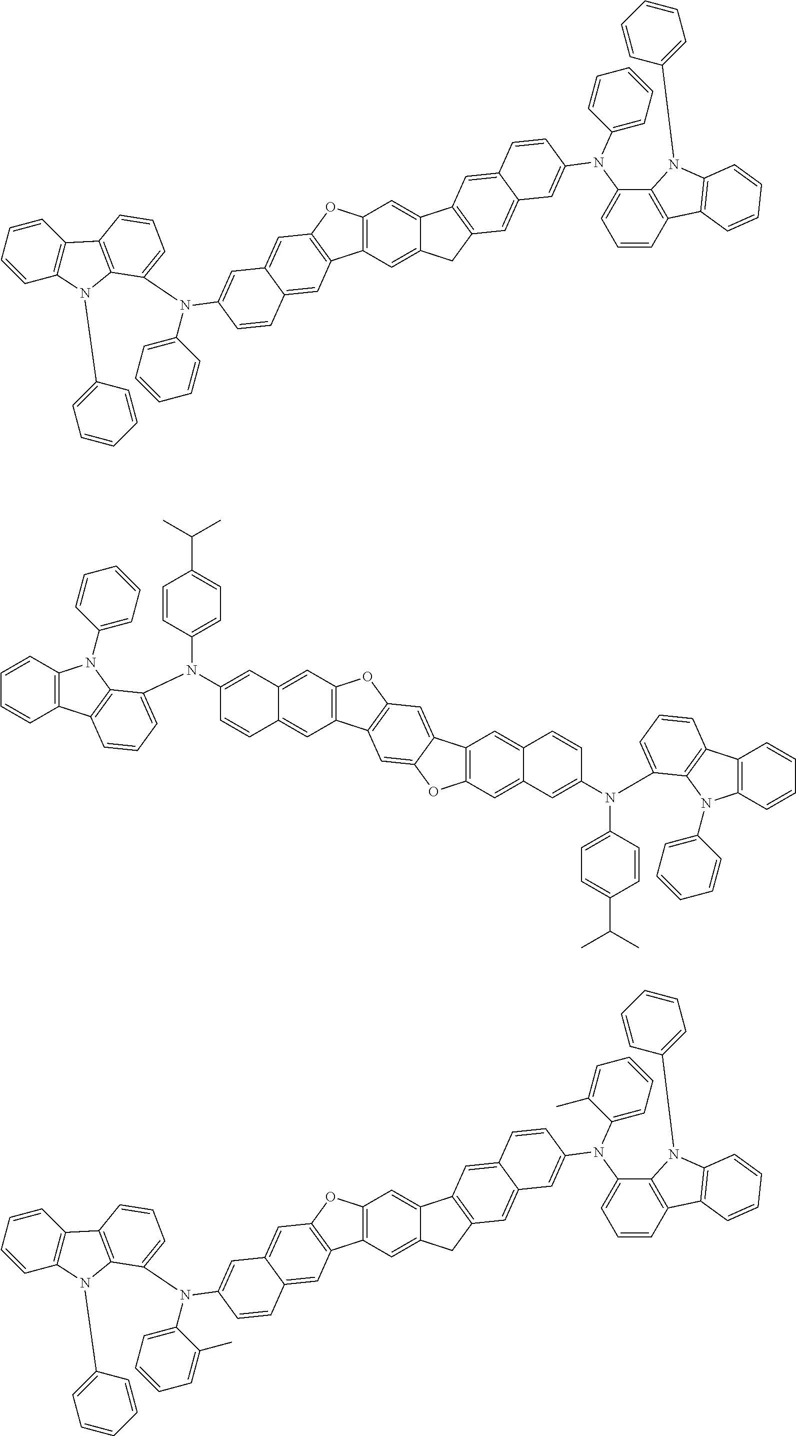

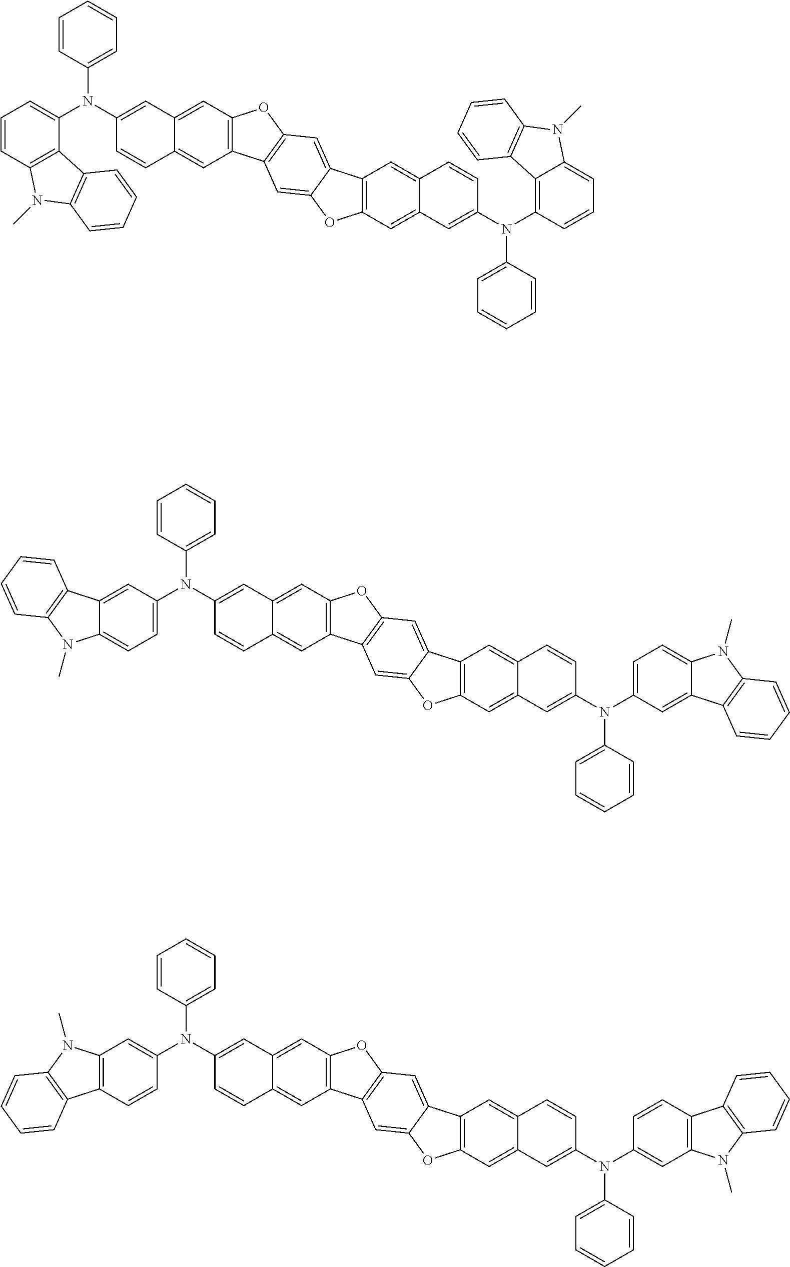 [SMILES: Cn1c2ccccc2c2c(N(c3ccccc3)c3ccc4cc5c(cc4c3)oc3cc4c(cc35)oc3cc5cc(N(c6ccccc6)c6cccc7c6c6ccccc6n7C)ccc5cc34)cccc21 ; Cn1c2ccccc2c2cc(N(c3ccccc3)c3ccc4cc5c(cc4c3)oc3cc4c(cc35)oc3cc5cc(N(c6ccccc6)c6ccc7c(c6)c6ccccc6n7C)ccc5cc34)ccc21 ; Cn1c2ccccc2c2ccc(N(c3ccccc3)c3ccc4cc5c(cc4c3)oc3cc4c(cc35)oc3cc5cc(N(c6ccccc6)c6ccc7c8ccccc8n(C)c7c6)ccc5cc34)cc21]

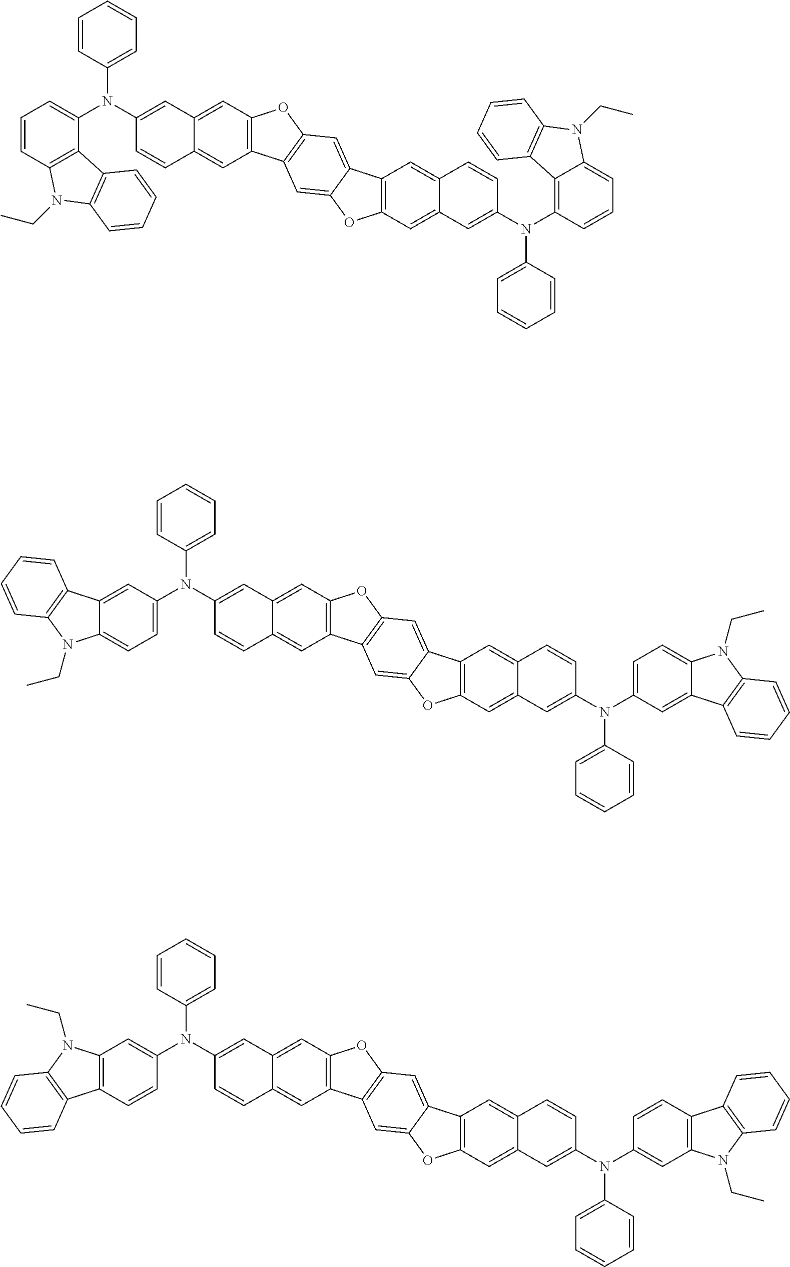 [SMILES: CCn1c2ccccc2c2c(N(c3ccccc3)c3ccc4cc5c(cc4c3)oc3cc4c(cc35)oc3cc5cc(N(c6ccccc6)c6cccc7c6c6ccccc6n7CC)ccc5cc34)cccc21 ; CCn1c2ccccc2c2cc(N(c3ccccc3)c3ccc4cc5c(cc4c3)oc3cc4c(cc35)oc3cc5cc(N(c6ccccc6)c6ccc7c(c6)c6ccccc6n7CC)ccc5cc34)ccc21 ; CCn1c2ccccc2c2ccc(N(c3ccccc3)c3ccc4cc5c(cc4c3)oc3cc4c(cc35)oc3cc5cc(N(c6ccccc6)c6ccc7c8ccccc8n(CC)c7c6)ccc5cc34)cc21]

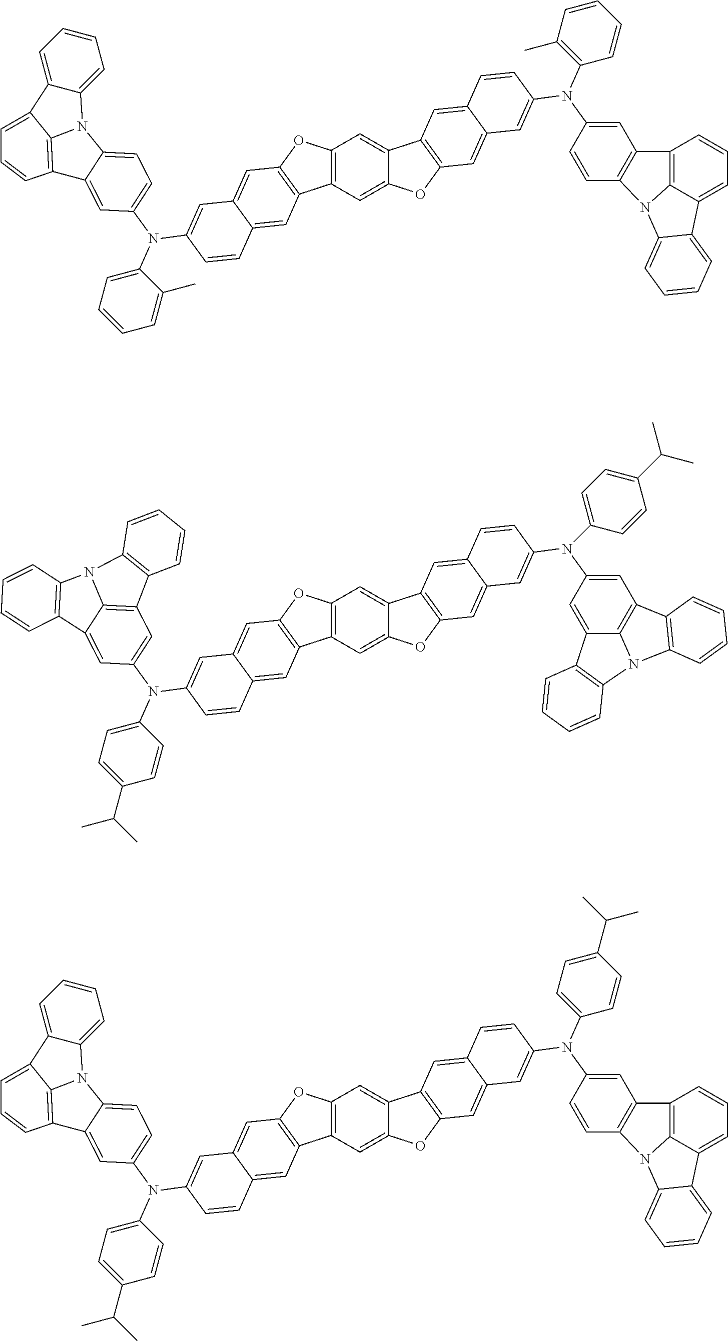 [SMILES: Cc1ccccc1N(c1ccc2cc3c(cc2c1)oc1cc2c(cc13)oc1cc3cc(N(c4ccc5c(c4)c4cccc6c7ccccc7n5c64)c4ccccc4C)ccc3cc12)c1ccc2c(c1)c1cccc3c4ccccc4n2c31 ; CC(C)c1ccc(N(c2ccc3cc4c(cc3c2)oc2cc3c(cc24)oc2cc4cc(N(c5ccc(C(C)C)cc5)c5cc6c7ccccc7n7c8ccccc8c(c5)c67)ccc4cc23)c2cc3c4ccccc4n4c5ccccc5c(c2)c34)cc1 ; CC(C)c1ccc(N(c2ccc3cc4c(cc3c2)oc2cc3c(cc24)oc2cc4cc(N(c5ccc(C(C)C)cc5)c5ccc6c(c5)c5cccc7c8ccccc8n6c75)ccc4cc23)c2ccc3c(c2)c2cccc4c5ccccc5n3c42)cc1]

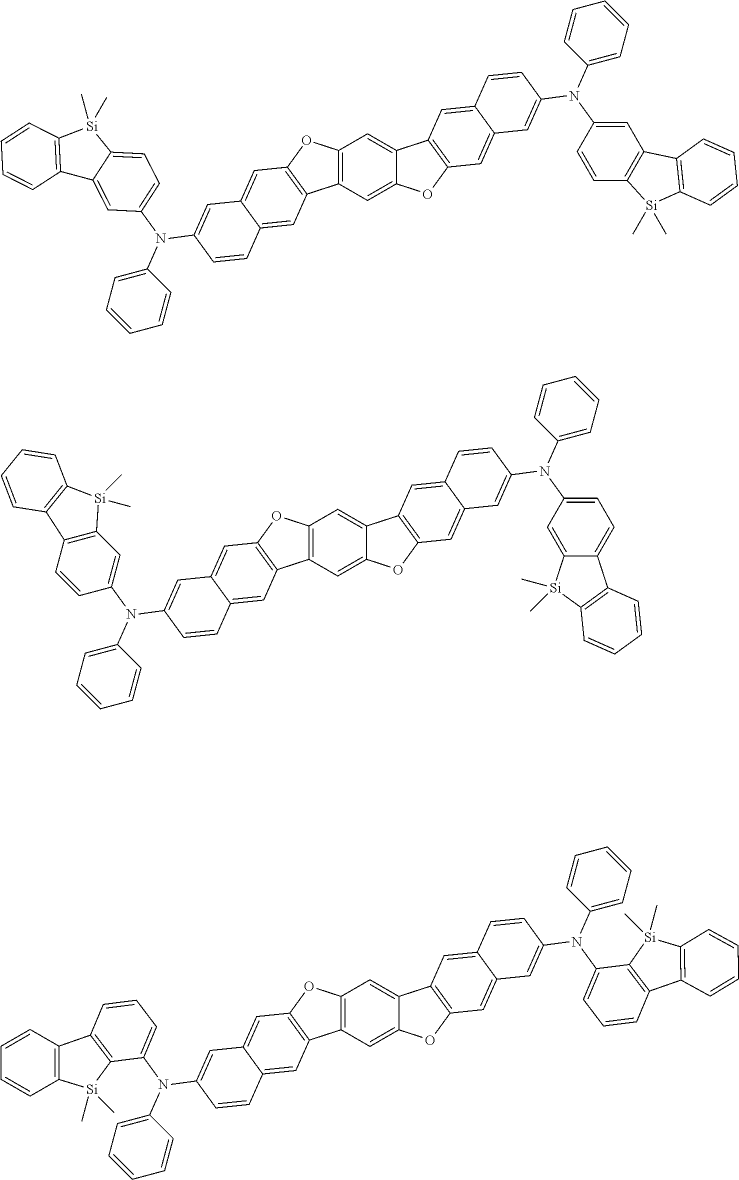 [SMILES: C[Si]1(C)c2ccccc2-c2cc(N(c3ccccc3)c3ccc4cc5c(cc4c3)oc3cc4c(cc35)oc3cc5cc(N(c6ccccc6)c6ccc7c(c6)-c6ccccc6[Si]7(C)C)ccc5cc34)ccc21 ; C[Si]1(C)c2ccccc2-c2ccc(N(c3ccccc3)c3ccc4cc5c(cc4c3)oc3cc4c(cc35)oc3cc5cc(N(c6ccccc6)c6ccc7c(c6)[Si](C)(C)c6ccccc6-7)ccc5cc34)cc21 ; C[Si]1(C)c2ccccc2-c2cccc(N(c3ccccc3)c3ccc4cc5c(cc4c3)oc3cc4c(cc35)oc3cc5cc(N(c6ccccc6)c6cccc7c6[Si](C)(C)c6ccccc6-7)ccc5cc34)c21]

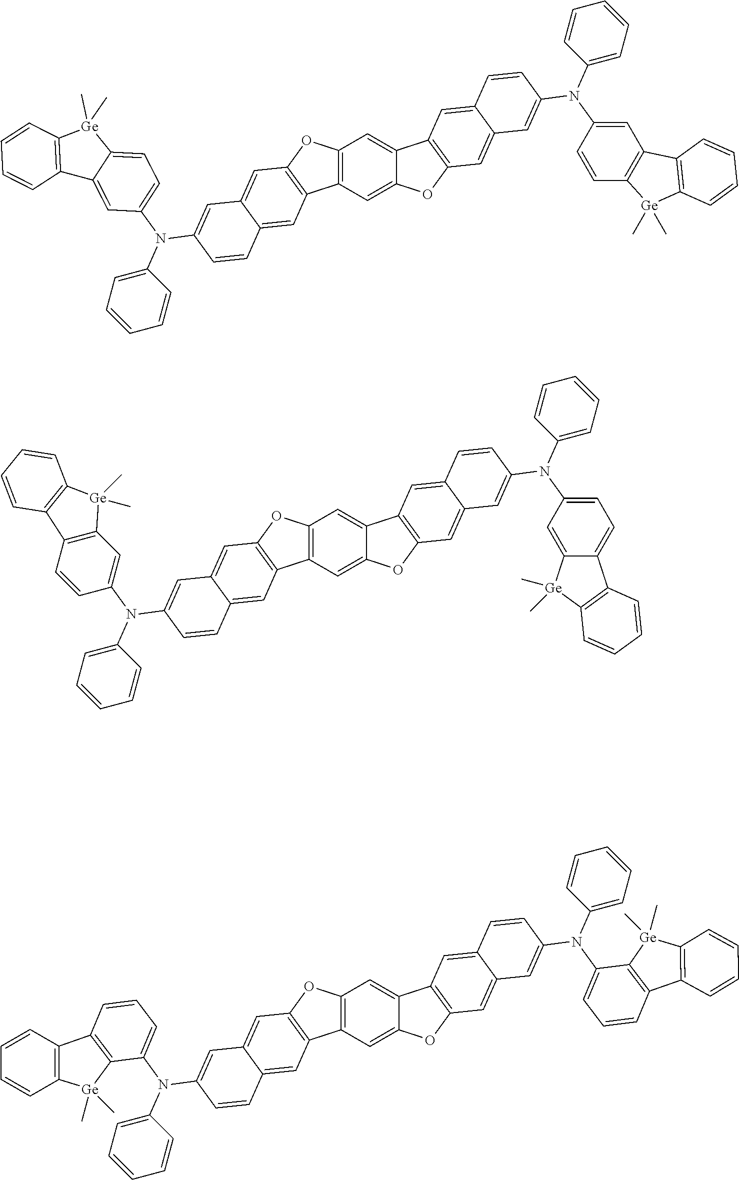

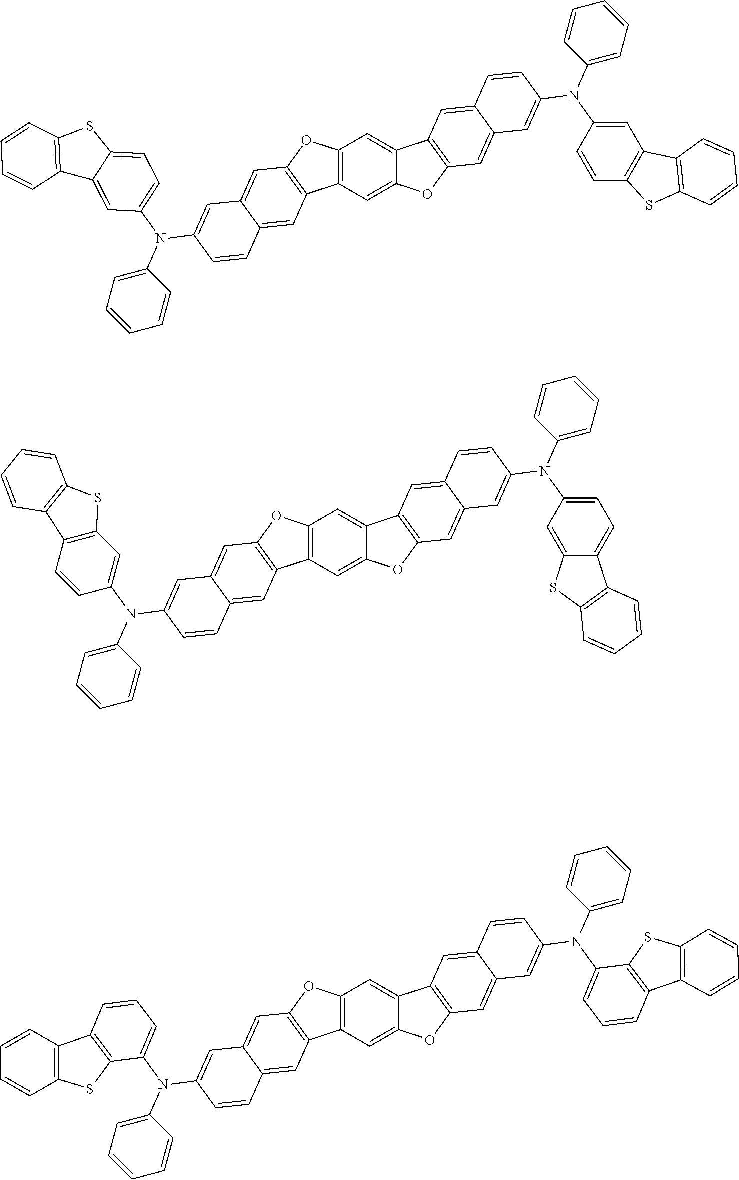 [SMILES: c1ccc(N(c2ccc3cc4c(cc3c2)oc2cc3c(cc24)oc2cc4cc(N(c5ccccc5)c5ccc6sc7ccccc7c6c5)ccc4cc23)c2ccc3sc4ccccc4c3c2)cc1 ; c1ccc(N(c2ccc3cc4c(cc3c2)oc2cc3c(cc24)oc2cc4cc(N(c5ccccc5)c5ccc6c(c5)sc5ccccc56)ccc4cc23)c2ccc3c(c2)sc2ccccc23)cc1 ; c1ccc(N(c2ccc3cc4c(cc3c2)oc2cc3c(cc24)oc2cc4cc(N(c5ccccc5)c5cccc6c5sc5ccccc56)ccc4cc23)c2cccc3c2sc2ccccc23)cc1]

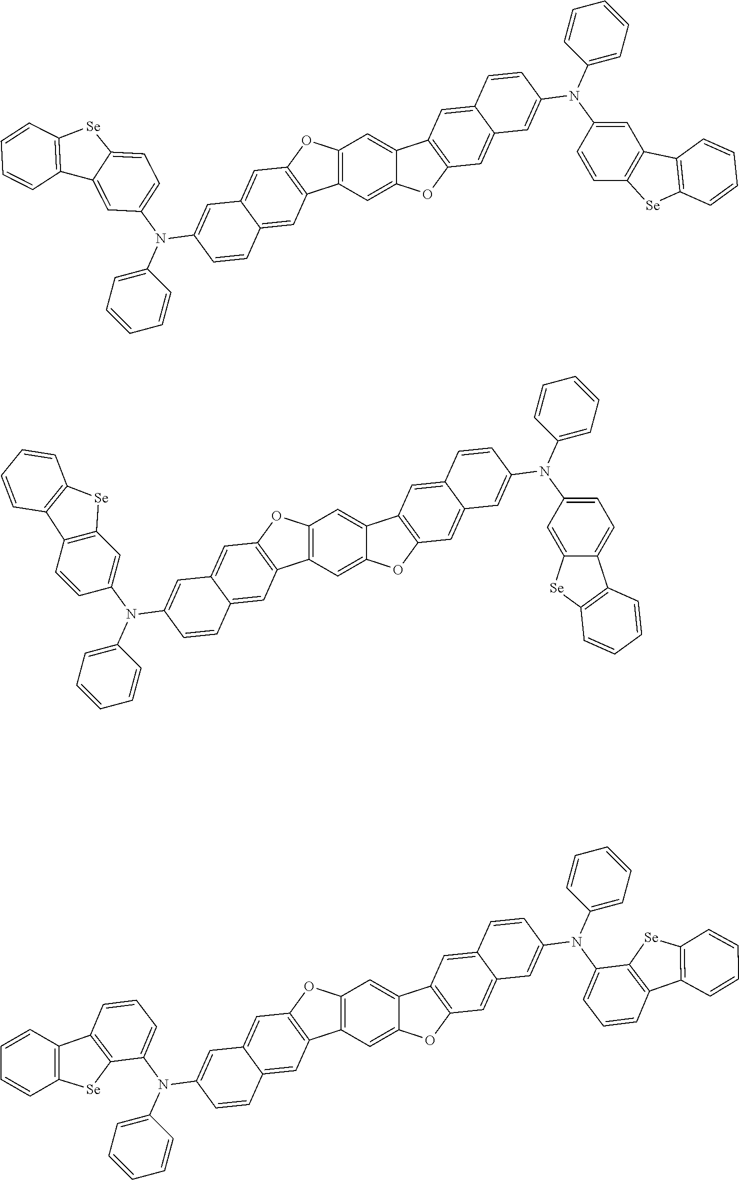 [SMILES: c1ccc(N(c2ccc3cc4c(cc3c2)oc2cc3c(cc24)oc2cc4cc(N(c5ccccc5)c5ccc6[se]c7ccccc7c6c5)ccc4cc23)c2ccc3[se]c4ccccc4c3c2)cc1 ; c1ccc(N(c2ccc3cc4c(cc3c2)oc2cc3c(cc24)oc2cc4cc(N(c5ccccc5)c5ccc6c(c5)[se]c5ccccc56)ccc4cc23)c2ccc3c(c2)[se]c2ccccc23)cc1 ; c1ccc(N(c2ccc3cc4c(cc3c2)oc2cc3c(cc24)oc2cc4cc(N(c5ccccc5)c5cccc6c5[se]c5ccccc56)ccc4cc23)c2cccc3c2[se]c2ccccc23)cc1]

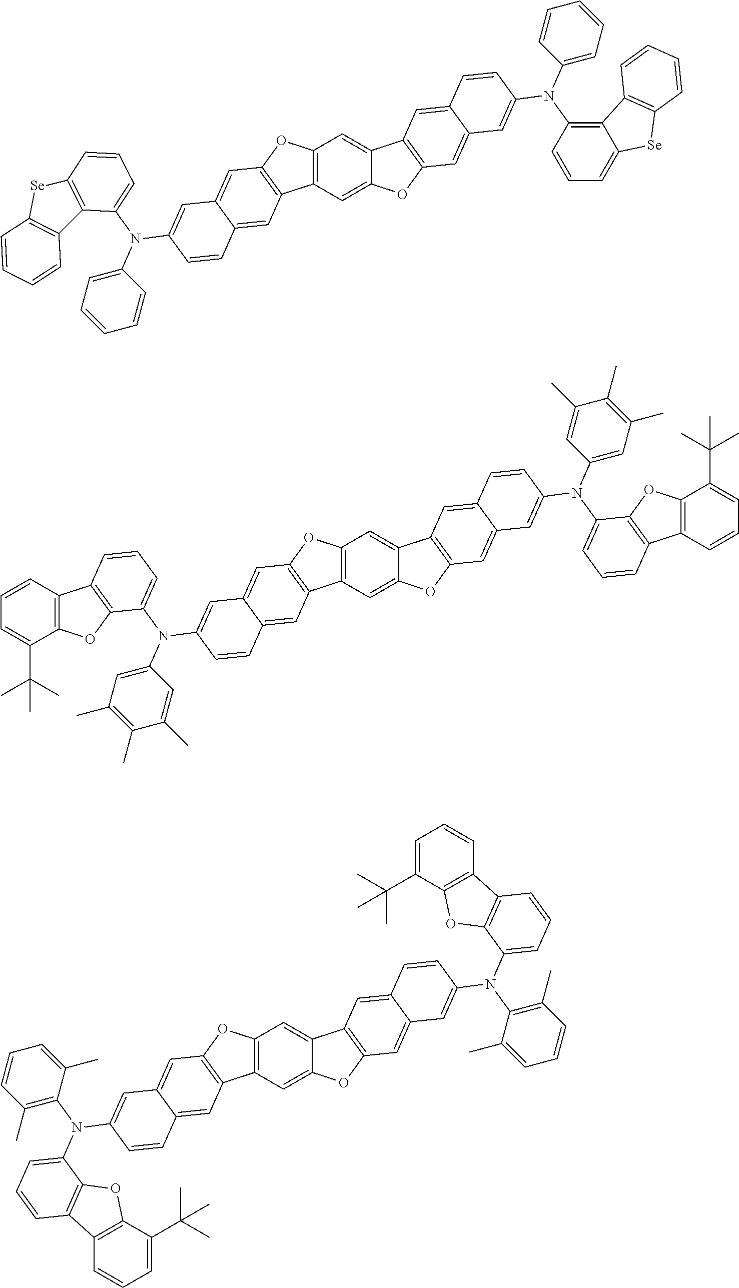 [SMILES: c1ccc(N(c2ccc3cc4c(cc3c2)oc2cc3c(cc24)oc2cc4cc(N(c5ccccc5)c5cccc6[se]c7ccccc7c56)ccc4cc23)c2cccc3[se]c4ccccc4c23)cc1 ; Cc1cc(N(c2ccc3cc4c(cc3c2)oc2cc3c(cc24)oc2cc4cc(N(c5cc(C)c(C)c(C)c5)c5cccc6c5oc5c(C(C)(C)C)cccc56)ccc4cc23)c2cccc3c2oc2c(C(C)(C)C)cccc23)cc(C)c1C ; Cc1cccc(C)c1N(c1ccc2cc3c(cc2c1)oc1cc2c(cc13)oc1cc3cc(N(c4c(C)cccc4C)c4cccc5c4oc4c(C(C)(C)C)cccc45)ccc3cc12)c1cccc2c1oc1c(C(C)(C)C)cccc12]

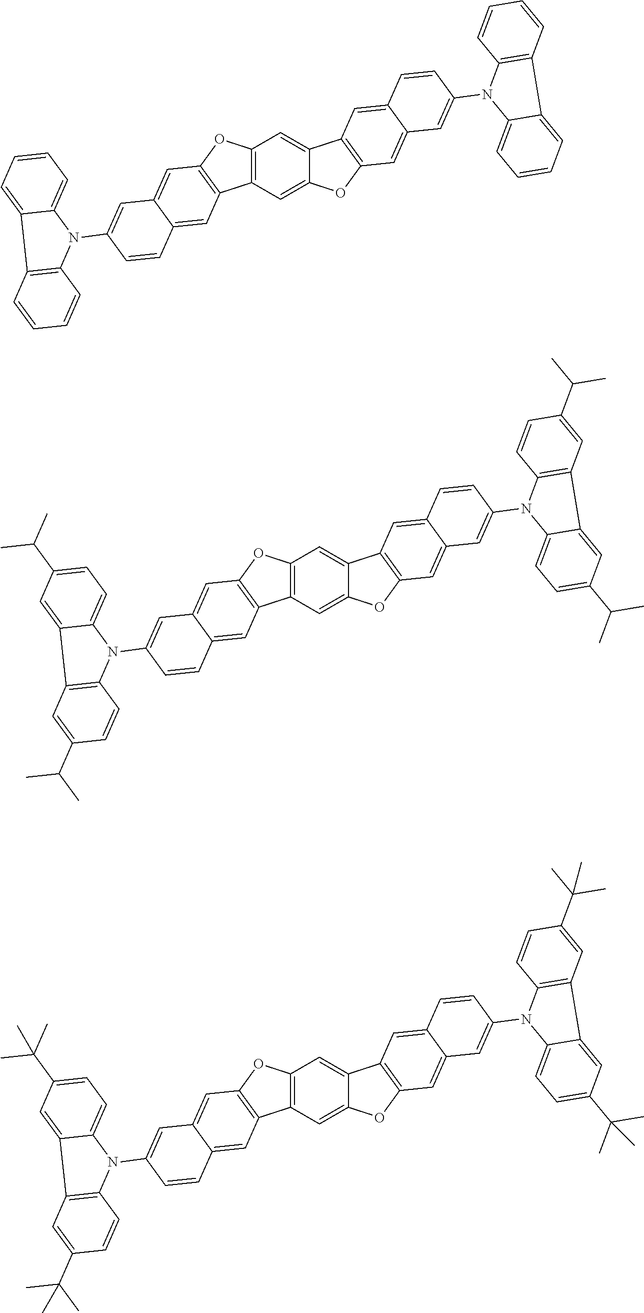

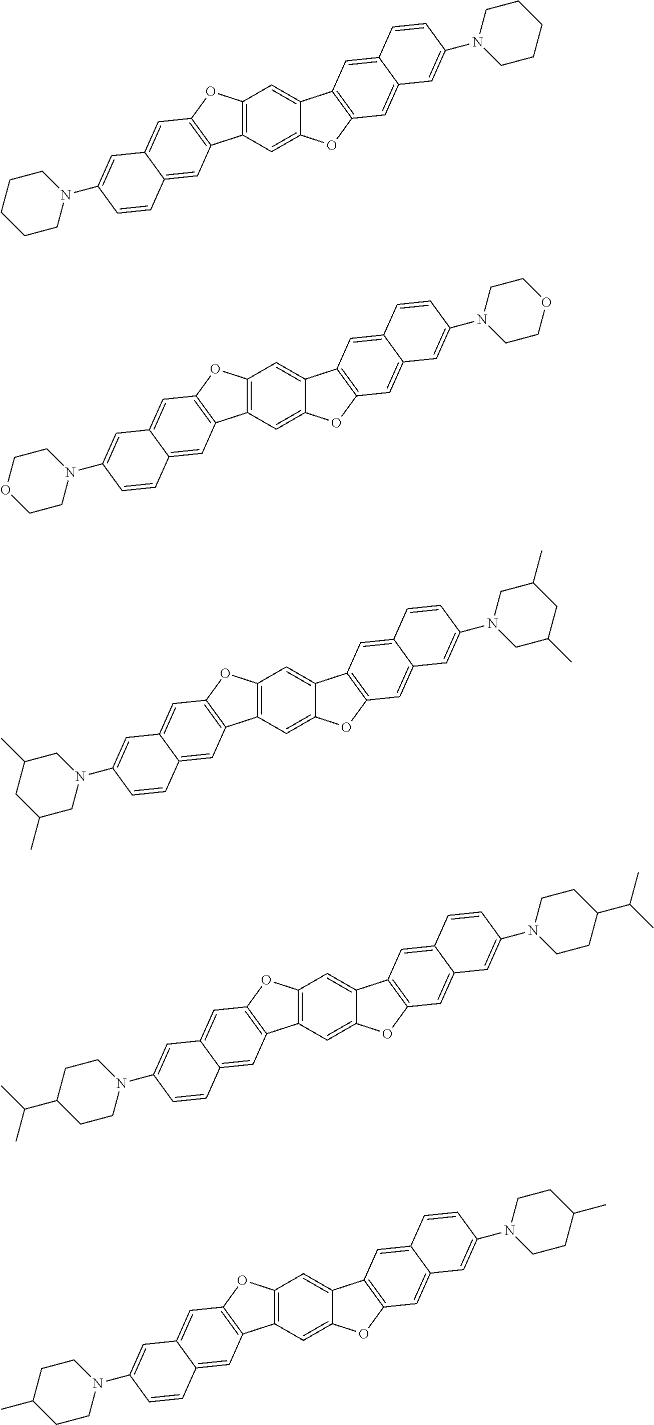

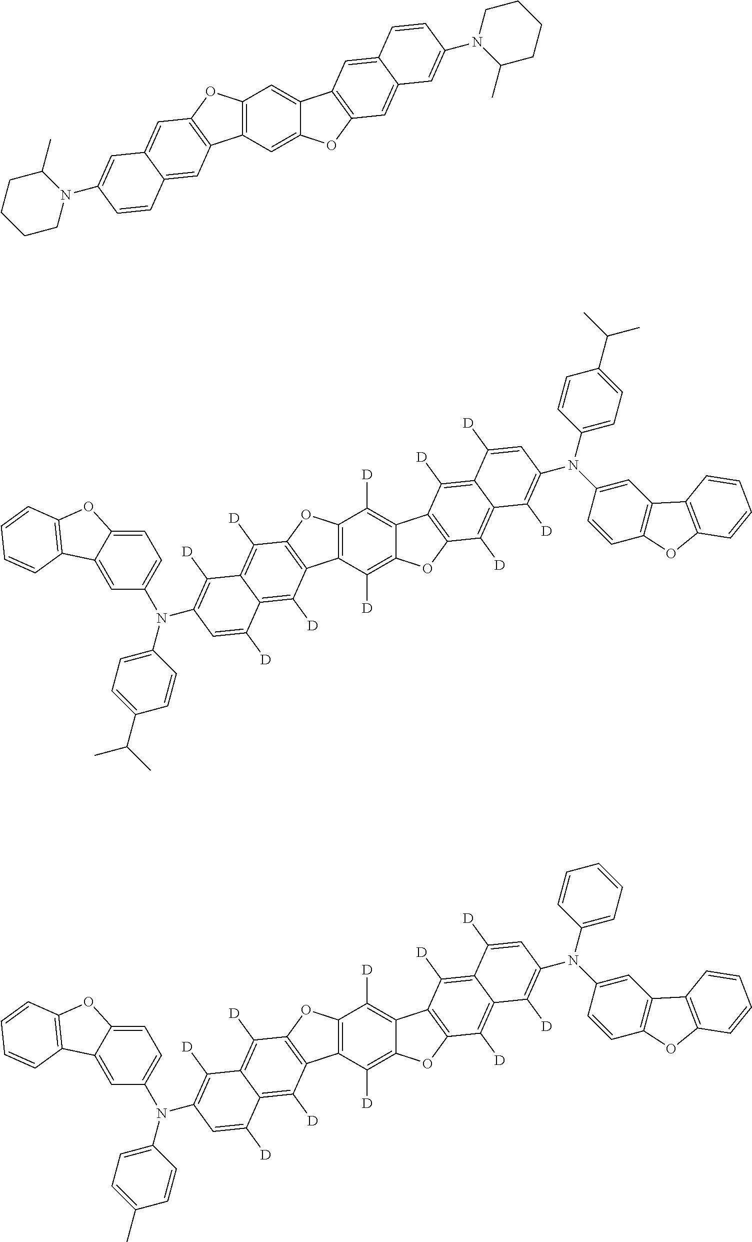 [SMILES: CC1CCCCN1c1ccc2cc3c(cc2c1)oc1cc2c(cc13)oc1cc3cc(N4CCCCC4C)ccc3cc12 ; [2H]c1cc(N(c2ccc(C(C)C)cc2)c2ccc3oc4ccccc4c3c2)c([2H])c2c([2H])c3oc4c([2H])c5c(oc6c([2H])c7c([2H])c(N(c8ccc(C(C)C)cc8)c8ccc9oc%10ccccc%10c9c8)cc([2H])c7c([2H])c65)c([2H])c4c3c([2H])c12 ; [2H]c1cc(N(c2ccccc2)c2ccc3oc4ccccc4c3c2)c([2H])c2c([2H])c3oc4c([2H])c5c(oc6c([2H])c7c([2H])c(N(c8ccc(C)cc8)c8ccc9oc%10ccccc%10c9c8)cc([2H])c7c([2H])c65)c([2H])c4c3c([2H])c12]

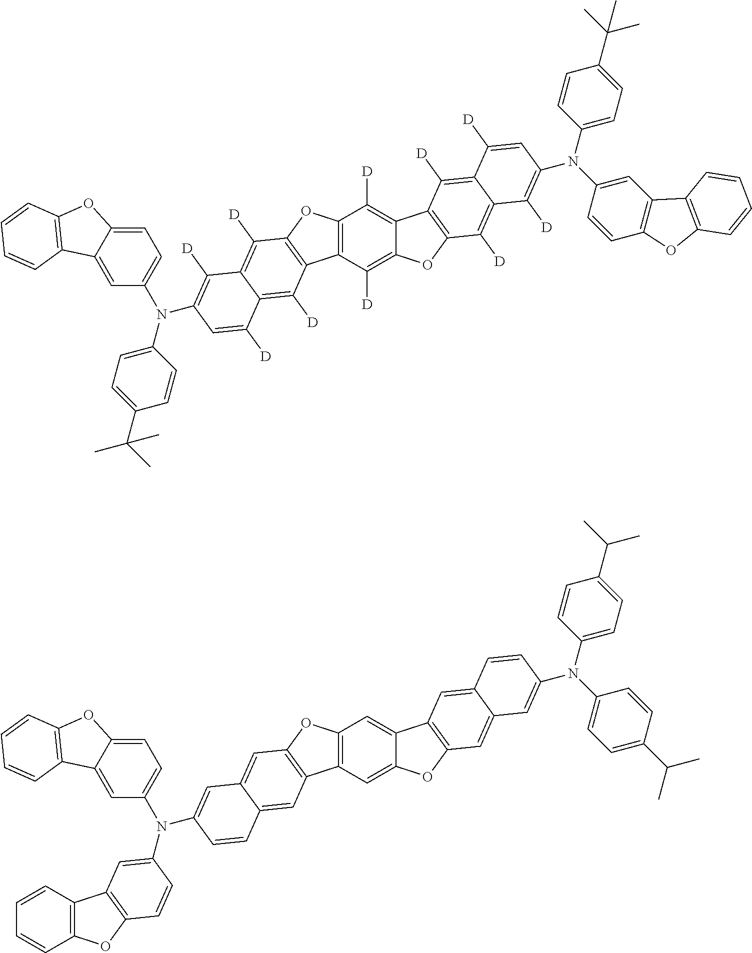 [SMILES: [2H]c1cc(N(c2ccc(C(C)(C)C)cc2)c2ccc3oc4ccccc4c3c2)c([2H])c2c([2H])c3oc4c([2H])c5c(oc6c([2H])c7c([2H])c(N(c8ccc(C(C)(C)C)cc8)c8ccc9oc%10ccccc%10c9c8)cc([2H])c7c([2H])c65)c([2H])c4c3c([2H])c12 ; CC(C)c1ccc(N(c2ccc(C(C)C)cc2)c2ccc3cc4c(cc3c2)oc2cc3c(cc24)oc2cc4cc(N(c5ccc6oc7ccccc7c6c5)c5ccc6oc7ccccc7c6c5)ccc4cc23)cc1]

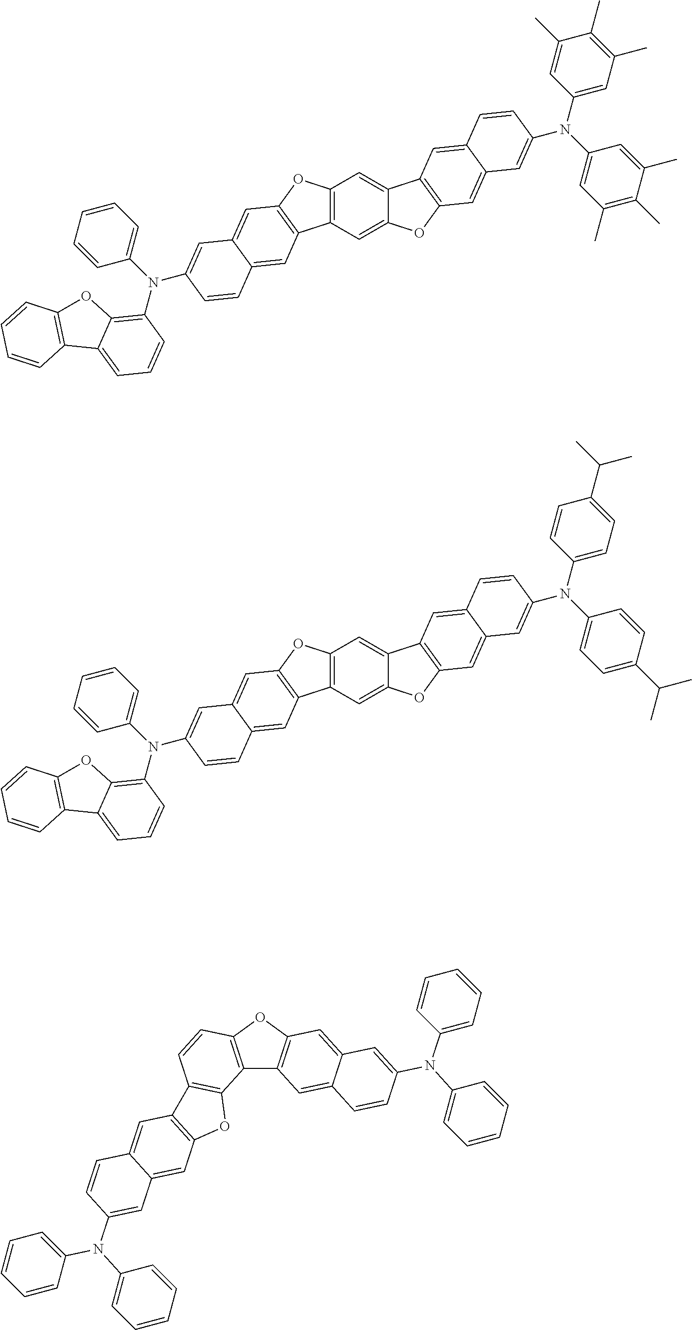 [SMILES: Cc1cc(N(c2cc(C)c(C)c(C)c2)c2ccc3cc4c(cc3c2)oc2cc3c(cc24)oc2cc4cc(N(c5ccccc5)c5cccc6c5oc5ccccc56)ccc4cc23)cc(C)c1C ; CC(C)c1ccc(N(c2ccc(C(C)C)cc2)c2ccc3cc4c(cc3c2)oc2cc3c(cc24)oc2cc4cc(N(c5ccccc5)c5cccc6c5oc5ccccc56)ccc4cc23)cc1 ; c1ccc(N(c2ccccc2)c2ccc3cc4c(cc3c2)oc2c4ccc3oc4cc5cc(N(c6ccccc6)c6ccccc6)ccc5cc4c32)cc1]

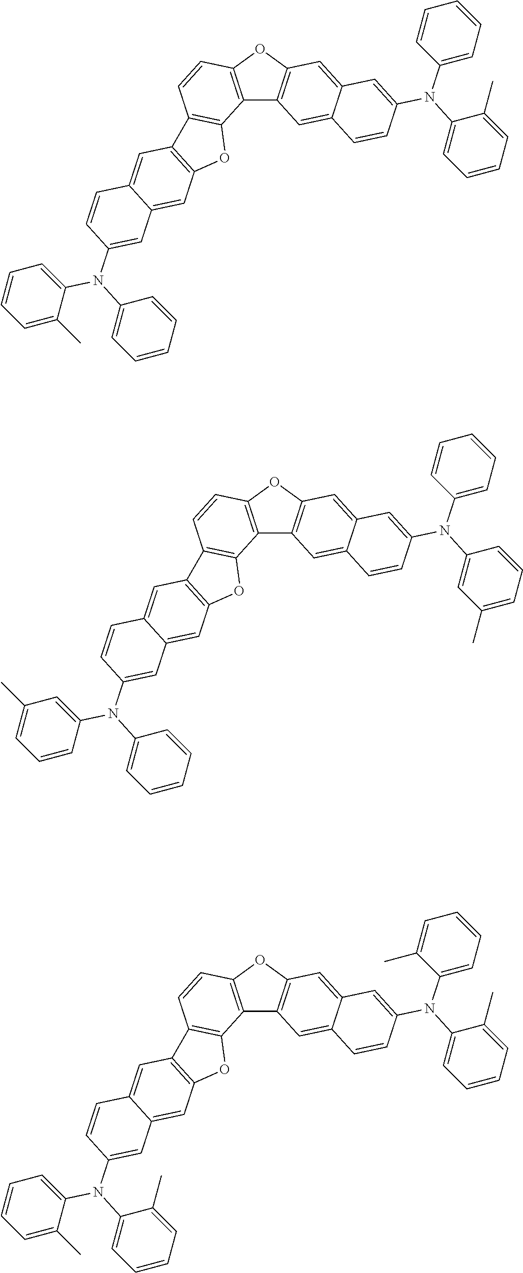 [SMILES: Cc1ccccc1N(c1ccccc1)c1ccc2cc3c(cc2c1)oc1c3ccc2oc3cc4cc(N(c5ccccc5)c5ccccc5C)ccc4cc3c21 ; Cc1cccc(N(c2ccccc2)c2ccc3cc4c(cc3c2)oc2c4ccc3oc4cc5cc(N(c6ccccc6)c6cccc(C)c6)ccc5cc4c32)c1 ; Cc1ccccc1N(c1ccc2cc3c(cc2c1)oc1c3ccc2oc3cc4cc(N(c5ccccc5C)c5ccccc5C)ccc4cc3c21)c1ccccc1C]

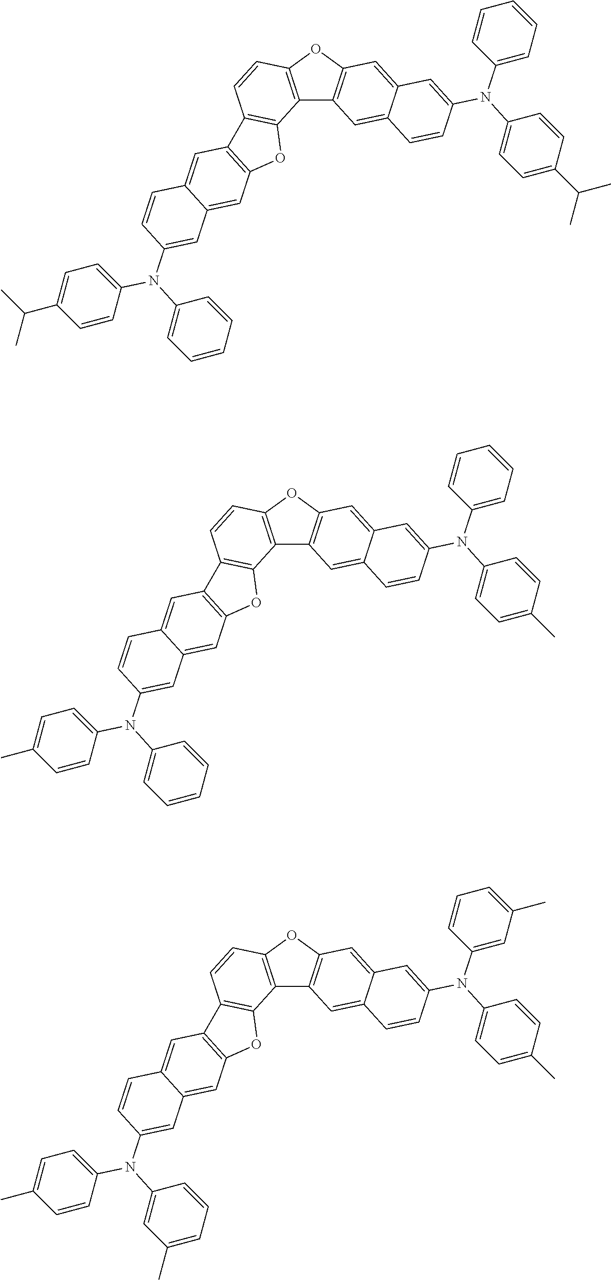

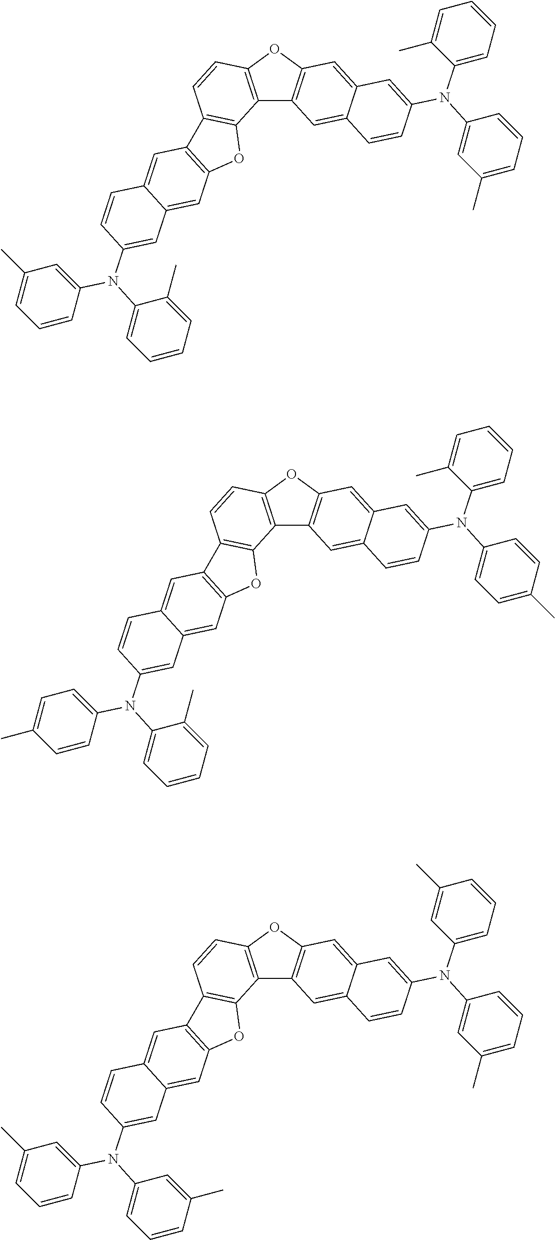

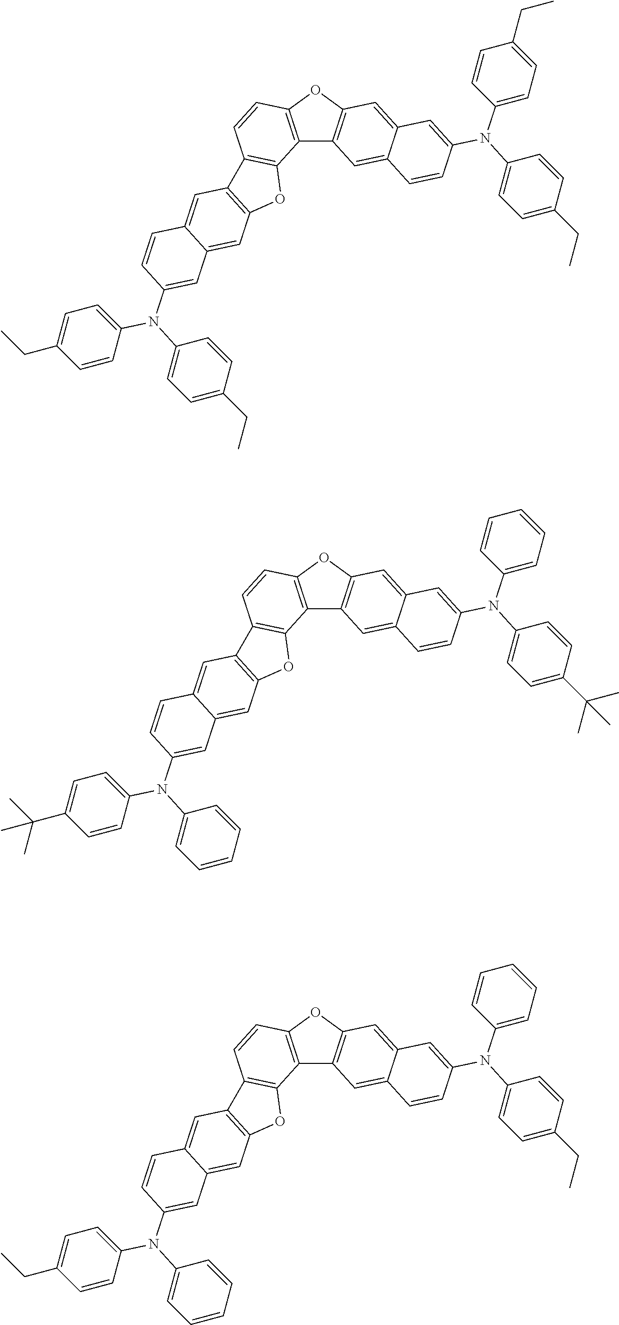

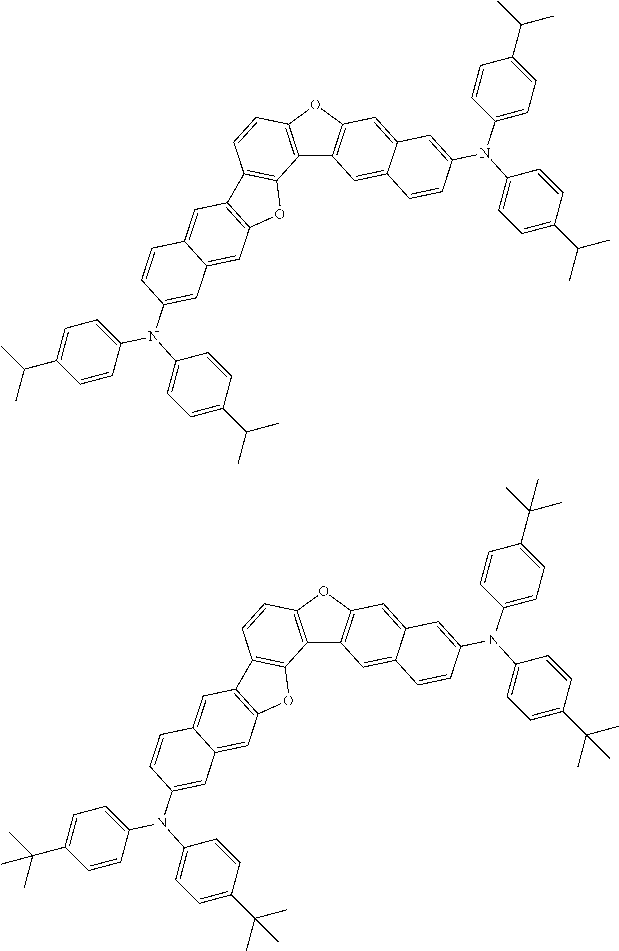

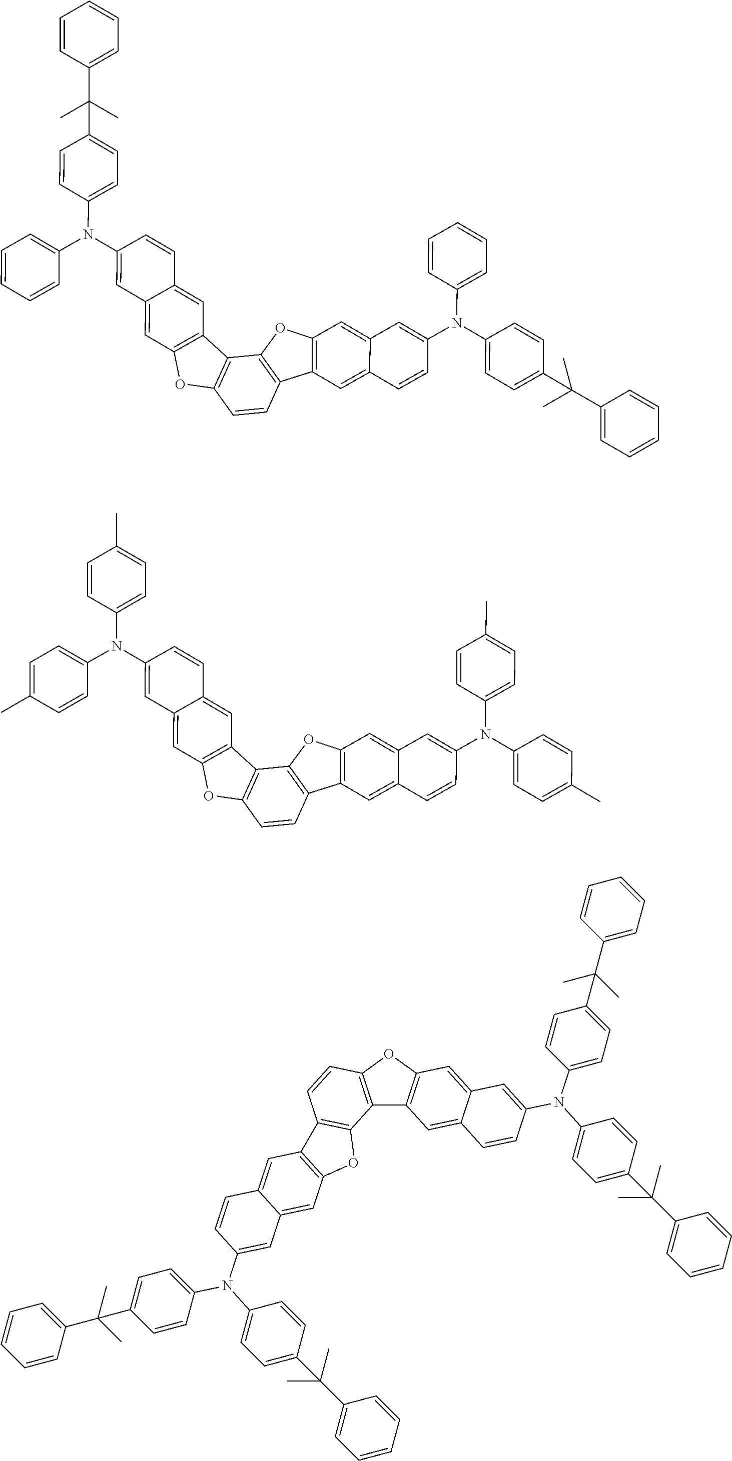

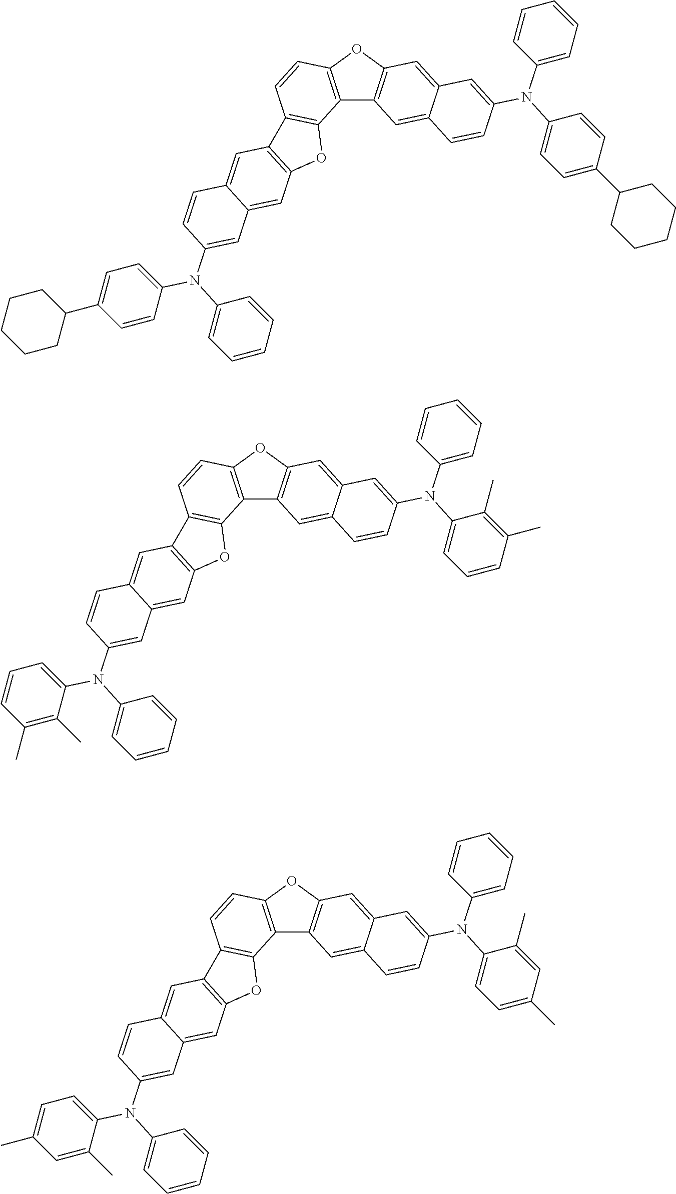

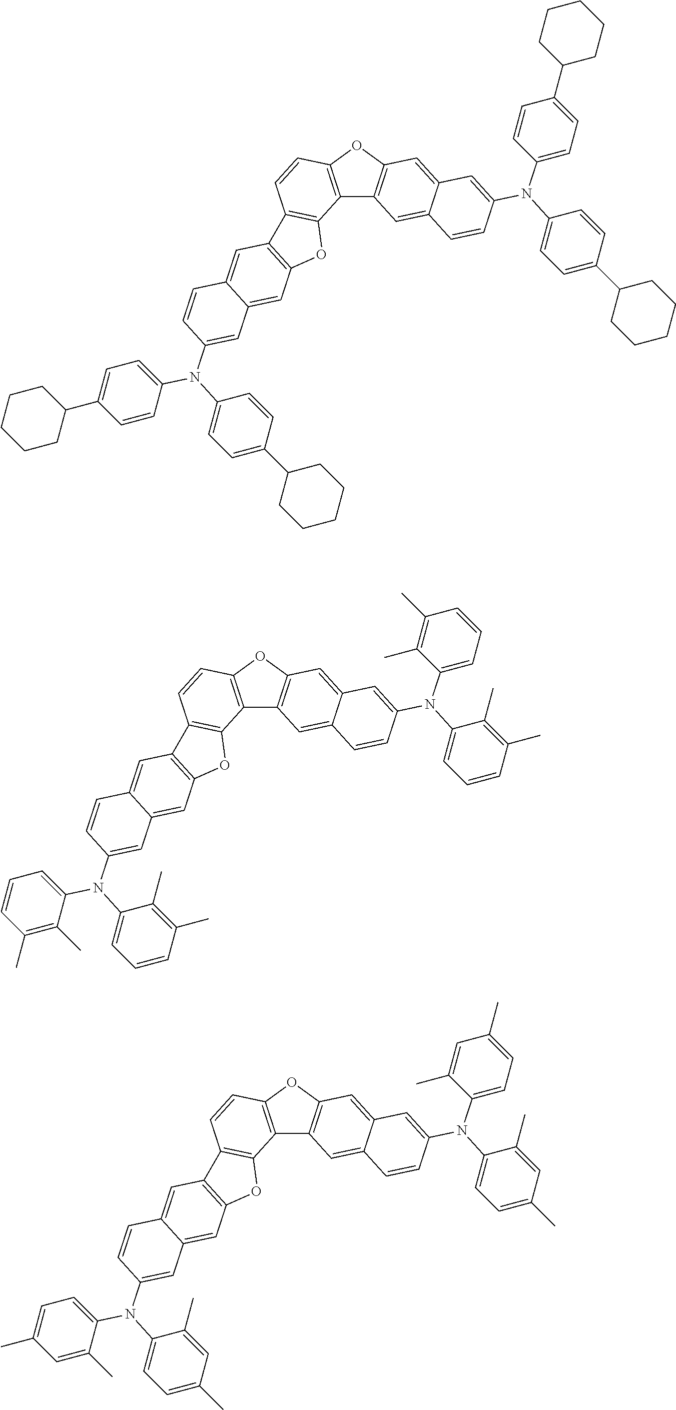

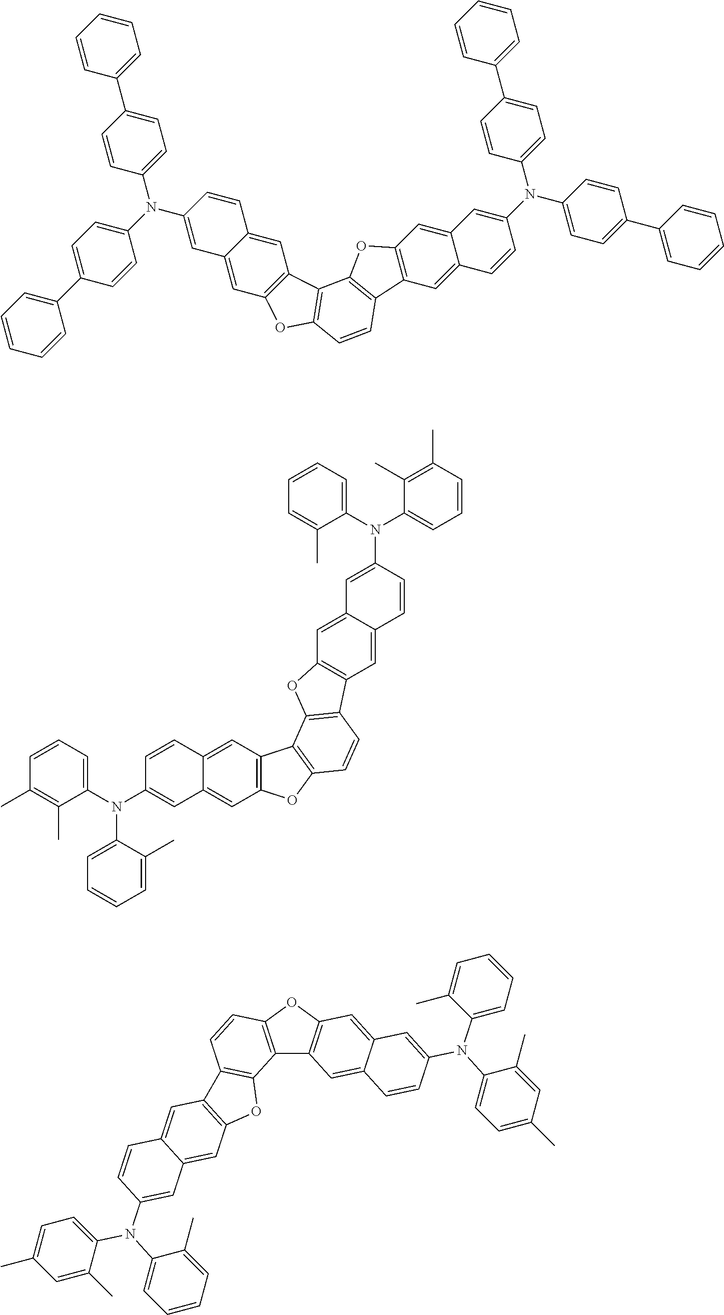

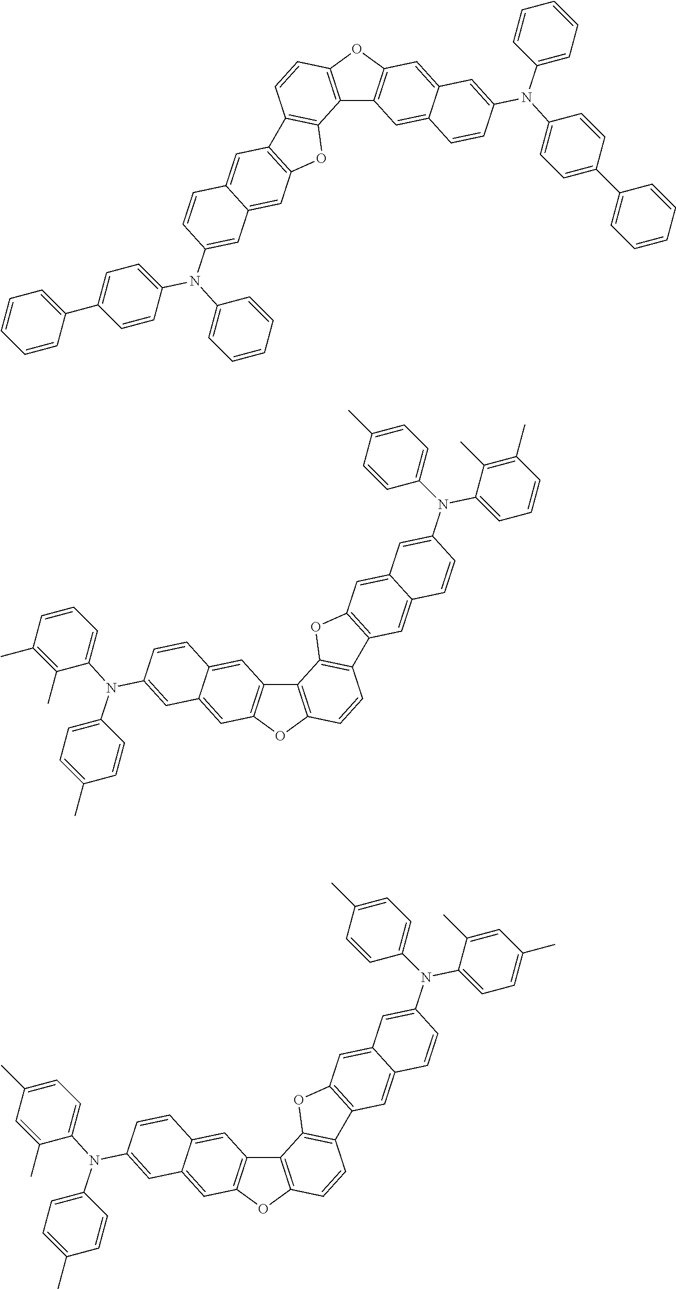

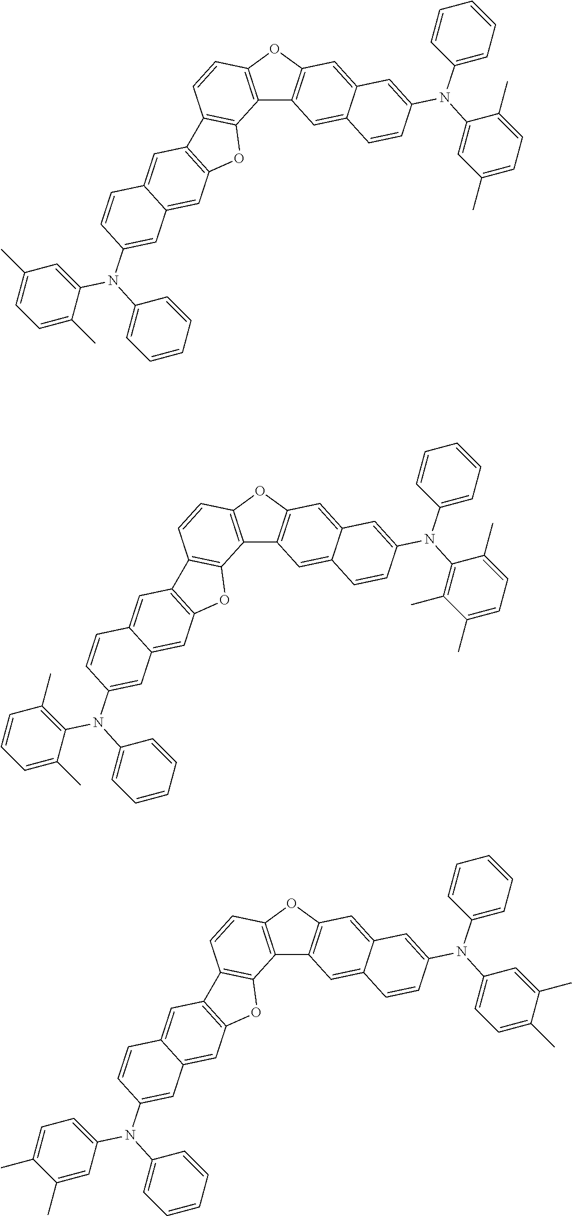

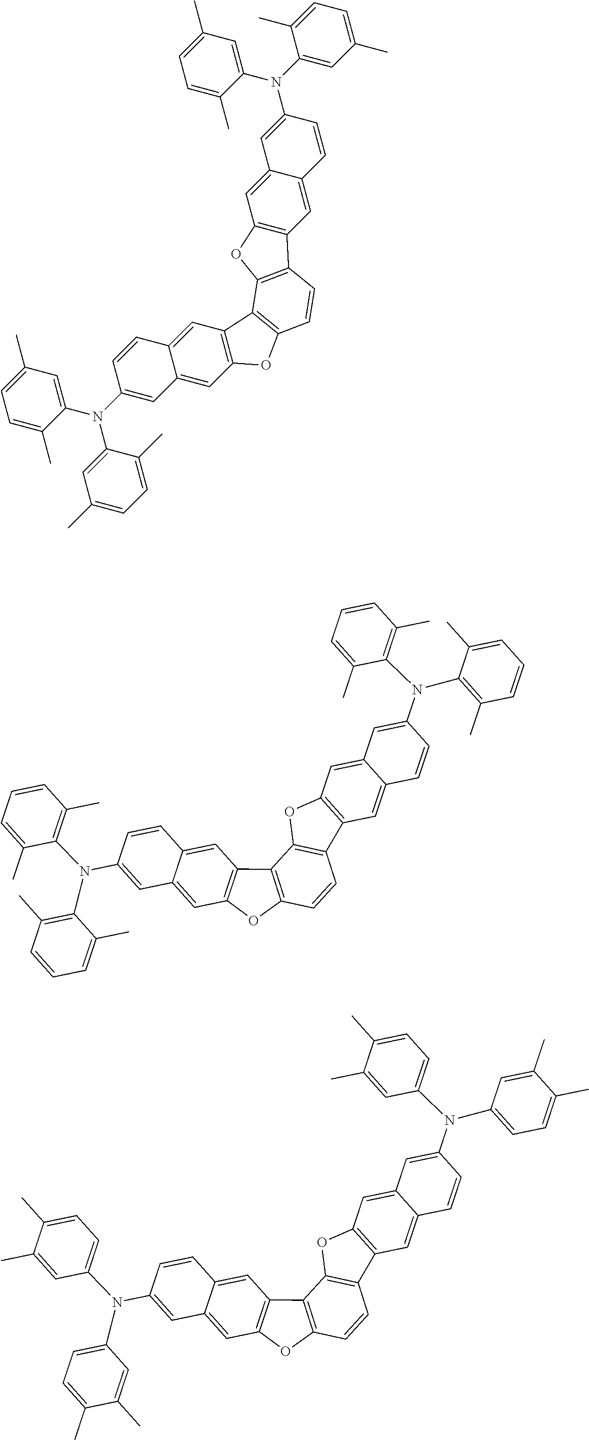

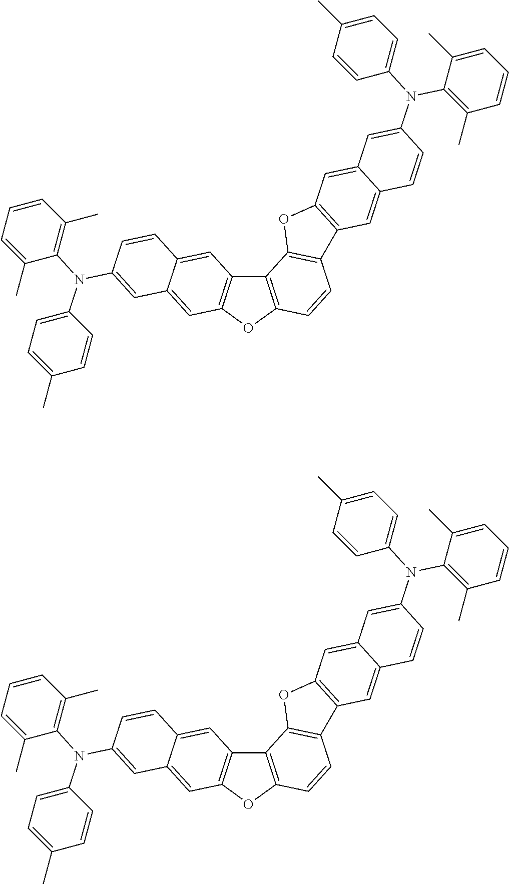 [SMILES: Cc1ccc(N(c2ccc3cc4c(cc3c2)oc2c4ccc3oc4cc5cc(N(c6ccc(C)cc6)c6c(C)cccc6C)ccc5cc4c32)c2c(C)cccc2C)cc1 ; Cc1ccc(N(c2ccc3cc4c(cc3c2)oc2c4ccc3oc4cc5cc(N(c6ccc(C)cc6)c6c(C)cccc6C)ccc5cc4c32)c2c(C)cccc2C)cc1]

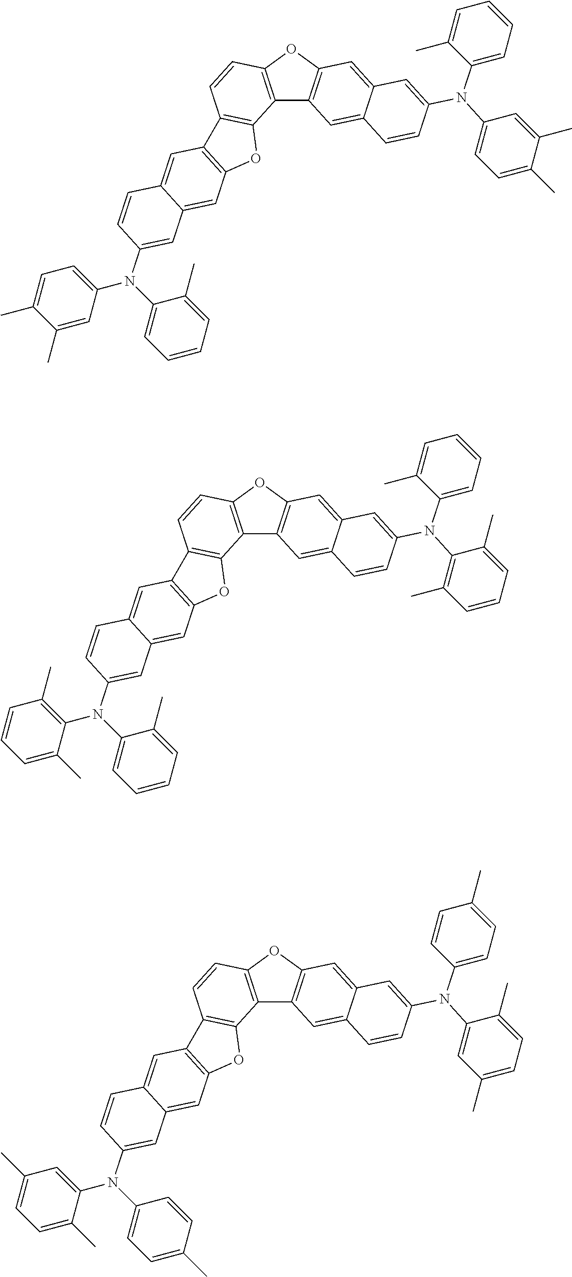

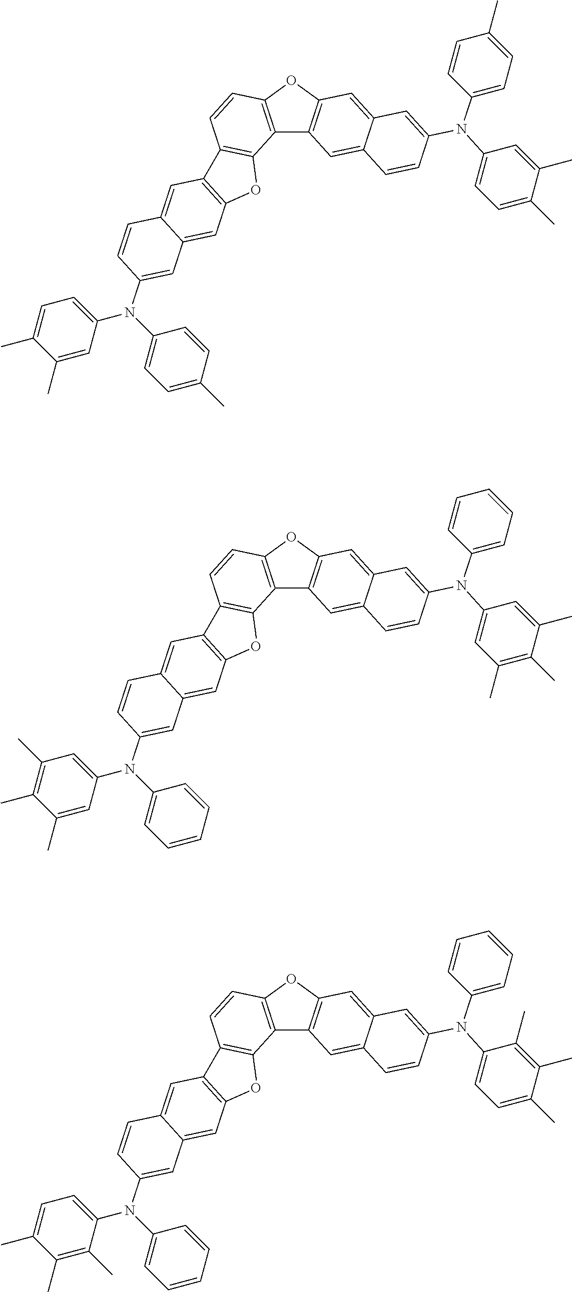

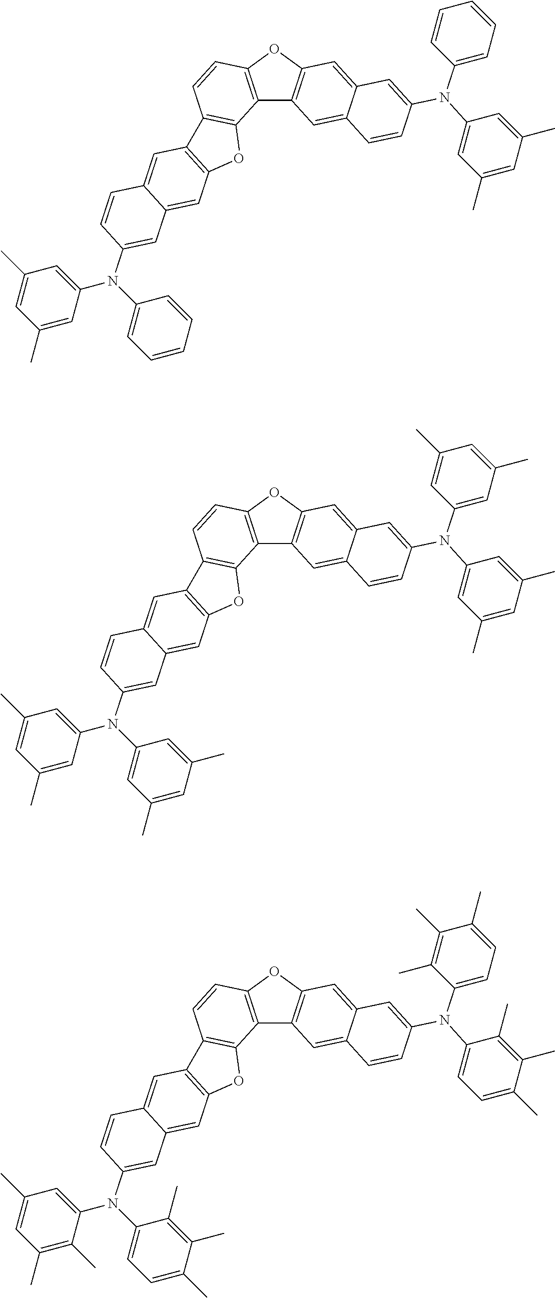

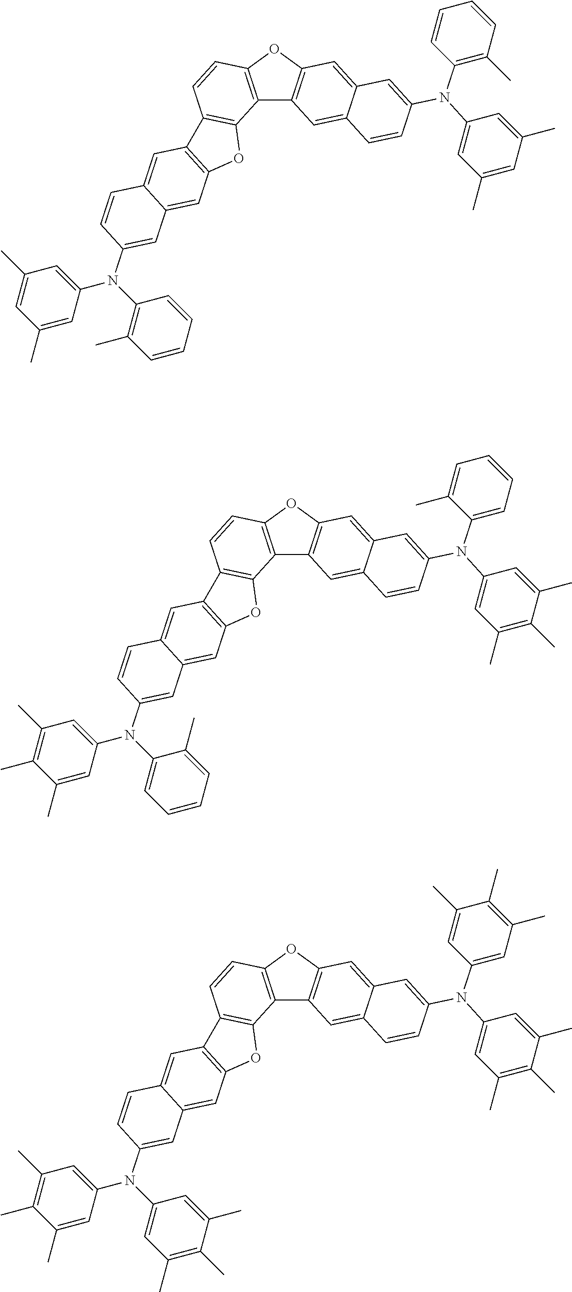

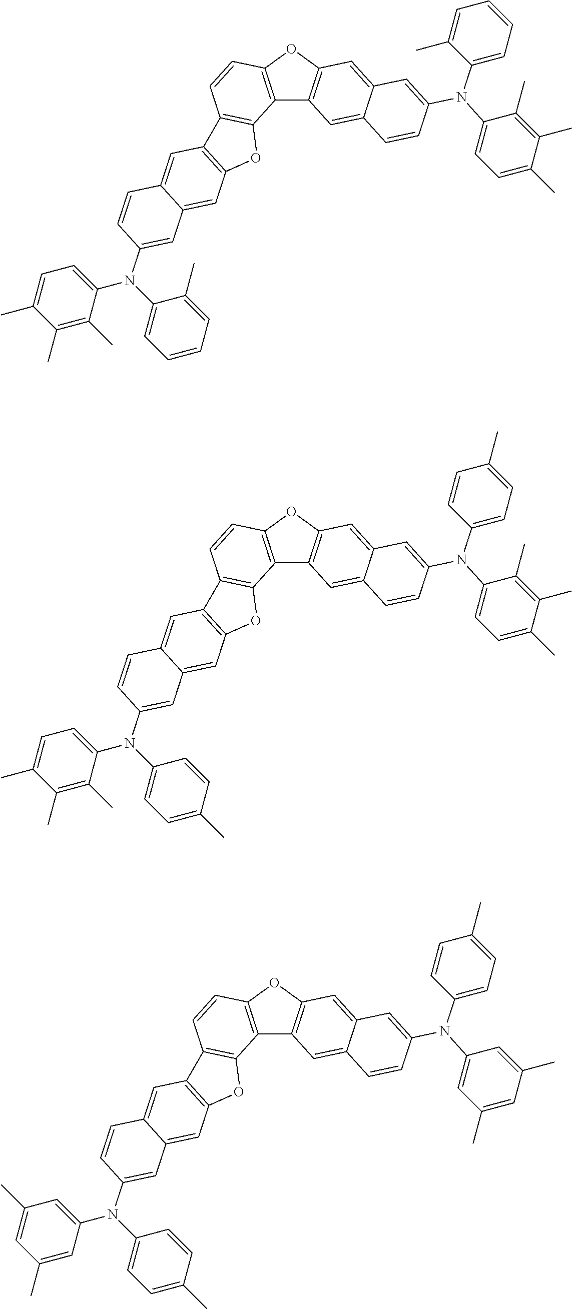

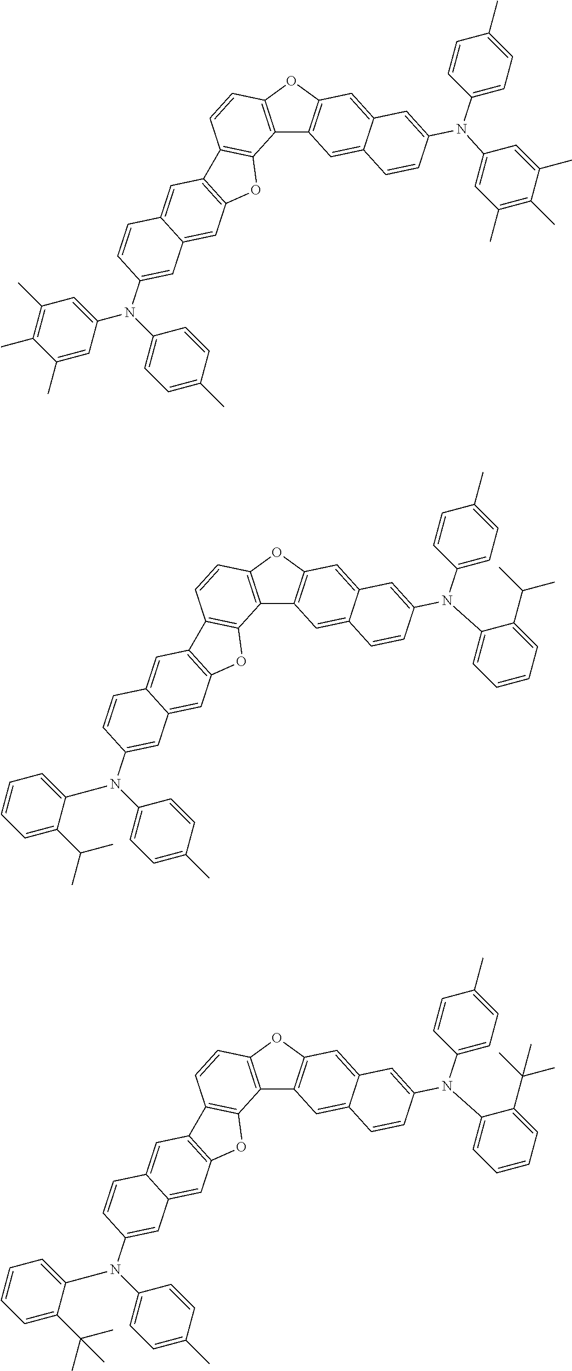

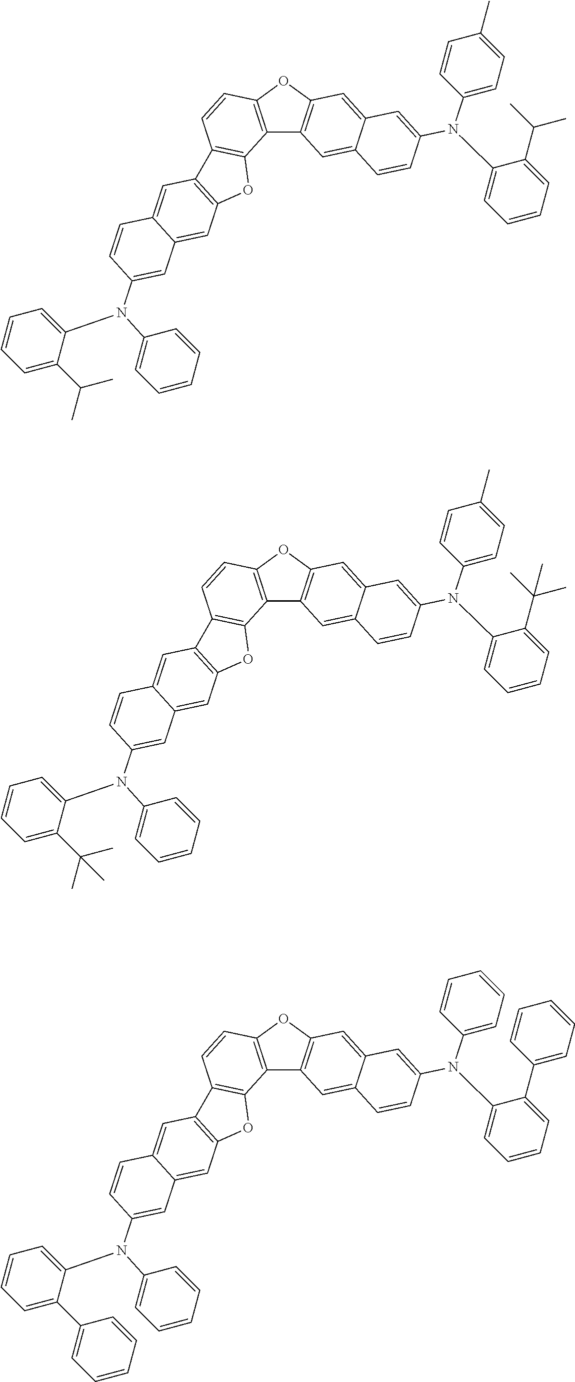 [SMILES: Cc1ccc(N(c2ccc3cc4c(cc3c2)oc2ccc3c5cc6ccc(N(c7ccccc7)c7ccccc7C(C)C)cc6cc5oc3c24)c2ccccc2C(C)C)cc1 ; Cc1ccc(N(c2ccc3cc4c(cc3c2)oc2ccc3c5cc6ccc(N(c7ccccc7)c7ccccc7C(C)(C)C)cc6cc5oc3c24)c2ccccc2C(C)(C)C)cc1 ; c1ccc(-c2ccccc2N(c2ccccc2)c2ccc3cc4c(cc3c2)oc2c4ccc3oc4cc5cc(N(c6ccccc6)c6ccccc6-c6ccccc6)ccc5cc4c32)cc1]

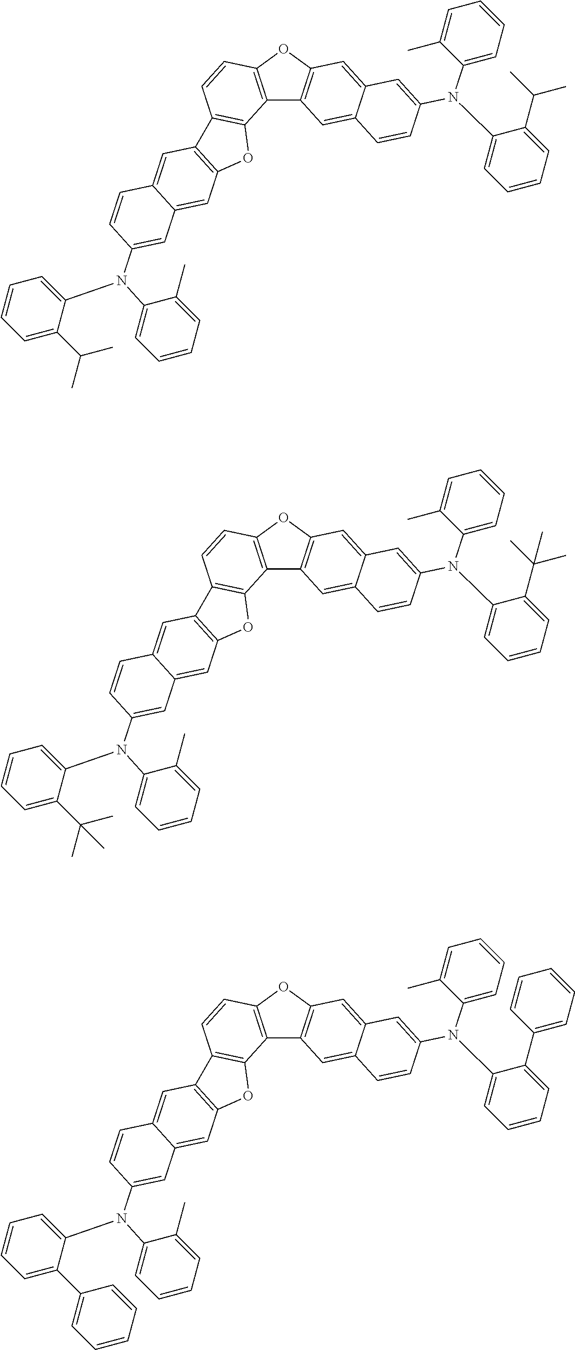 [SMILES: Cc1ccccc1N(c1ccc2cc3c(cc2c1)oc1c3ccc2oc3cc4cc(N(c5ccccc5C)c5ccccc5C(C)C)ccc4cc3c21)c1ccccc1C(C)C ; Cc1ccccc1N(c1ccc2cc3c(cc2c1)oc1c3ccc2oc3cc4cc(N(c5ccccc5C)c5ccccc5C(C)(C)C)ccc4cc3c21)c1ccccc1C(C)(C)C ; Cc1ccccc1N(c1ccc2cc3c(cc2c1)oc1c3ccc2oc3cc4cc(N(c5ccccc5C)c5ccccc5-c5ccccc5)ccc4cc3c21)c1ccccc1-c1ccccc1]

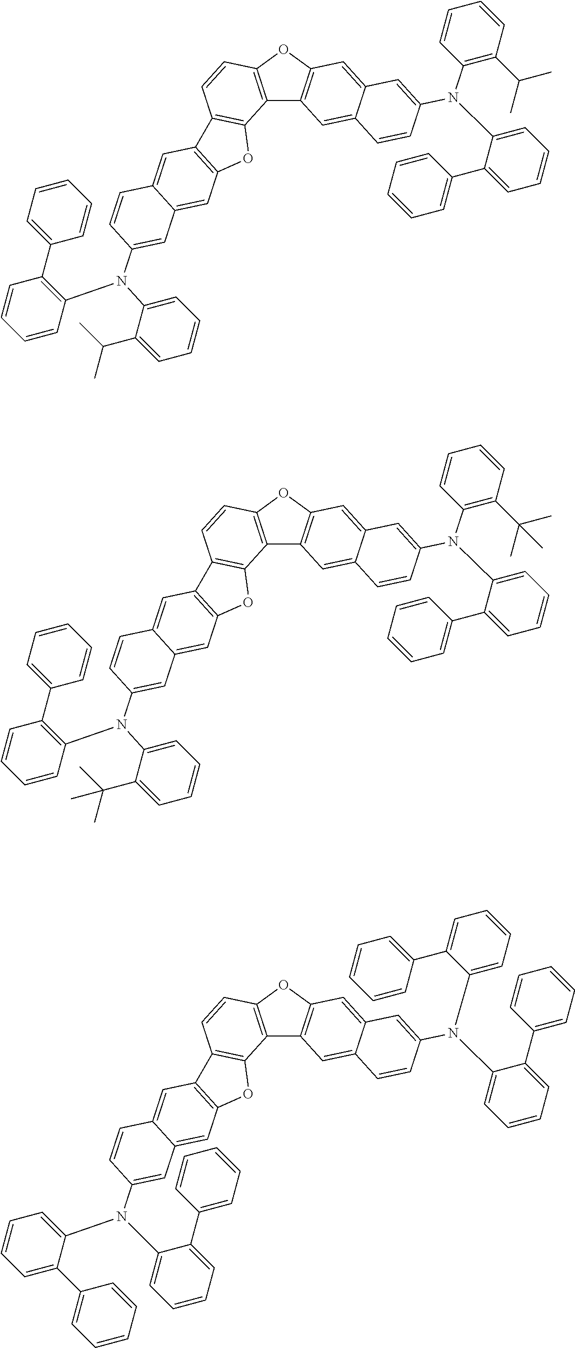 [SMILES: CC(C)c1ccccc1N(c1ccc2cc3c(cc2c1)oc1c3ccc2oc3cc4cc(N(c5ccccc5-c5ccccc5)c5ccccc5C(C)C)ccc4cc3c21)c1ccccc1-c1ccccc1 ; CC(C)(C)c1ccccc1N(c1ccc2cc3c(cc2c1)oc1c3ccc2oc3cc4cc(N(c5ccccc5-c5ccccc5)c5ccccc5C(C)(C)C)ccc4cc3c21)c1ccccc1-c1ccccc1 ; c1ccc(-c2ccccc2N(c2ccc3cc4c(cc3c2)oc2c4ccc3oc4cc5cc(N(c6ccccc6-c6ccccc6)c6ccccc6-c6ccccc6)ccc5cc4c32)c2ccccc2-c2ccccc2)cc1]

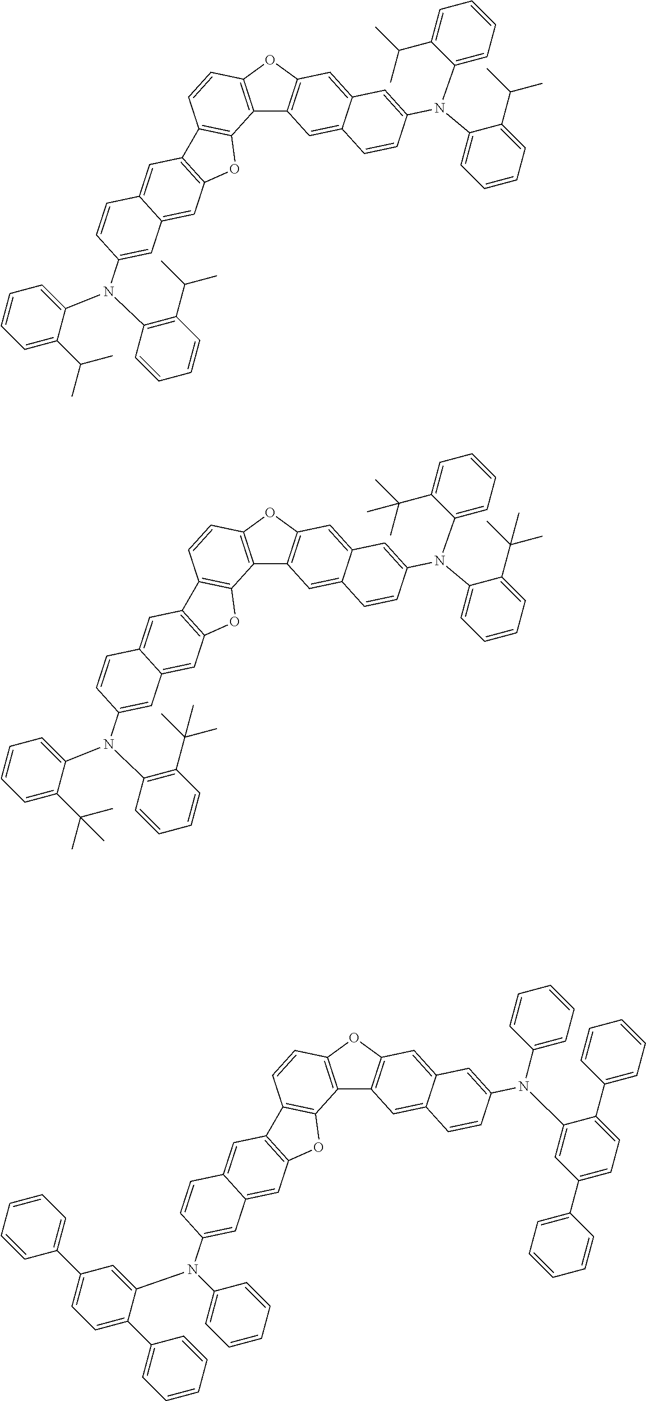 [SMILES: CC(C)c1ccccc1N(c1ccc2cc3c(cc2c1)oc1c3ccc2oc3cc4cc(N(c5ccccc5C(C)C)c5ccccc5C(C)C)ccc4cc3c21)c1ccccc1C(C)C ; CC(C)(C)c1ccccc1N(c1ccc2cc3c(cc2c1)oc1c3ccc2oc3cc4cc(N(c5ccccc5C(C)(C)C)c5ccccc5C(C)(C)C)ccc4cc3c21)c1ccccc1C(C)(C)C ; c1ccc(-c2ccc(-c3ccccc3)c(N(c3ccccc3)c3ccc4cc5c(cc4c3)oc3c5ccc4oc5cc6cc(N(c7ccccc7)c7cc(-c8ccccc8)ccc7-c7ccccc7)ccc6cc5c43)c2)cc1]

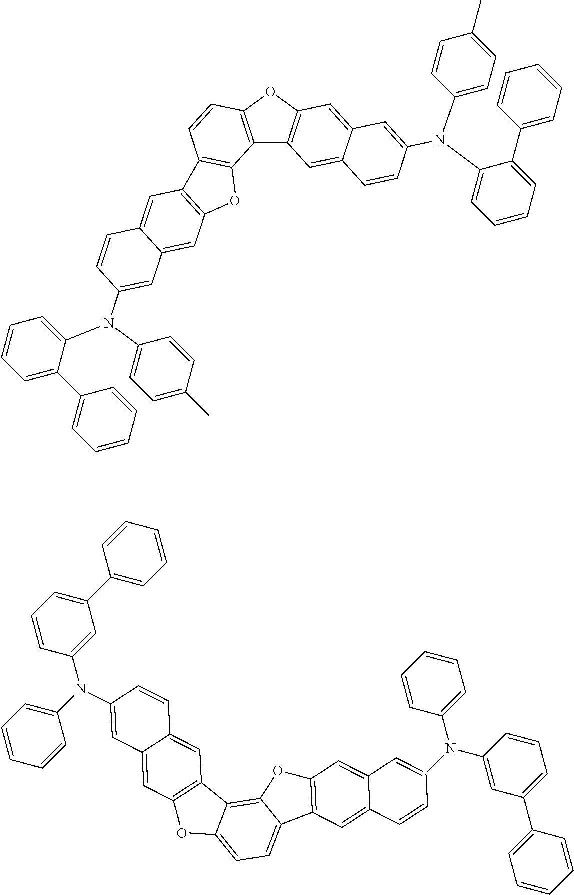

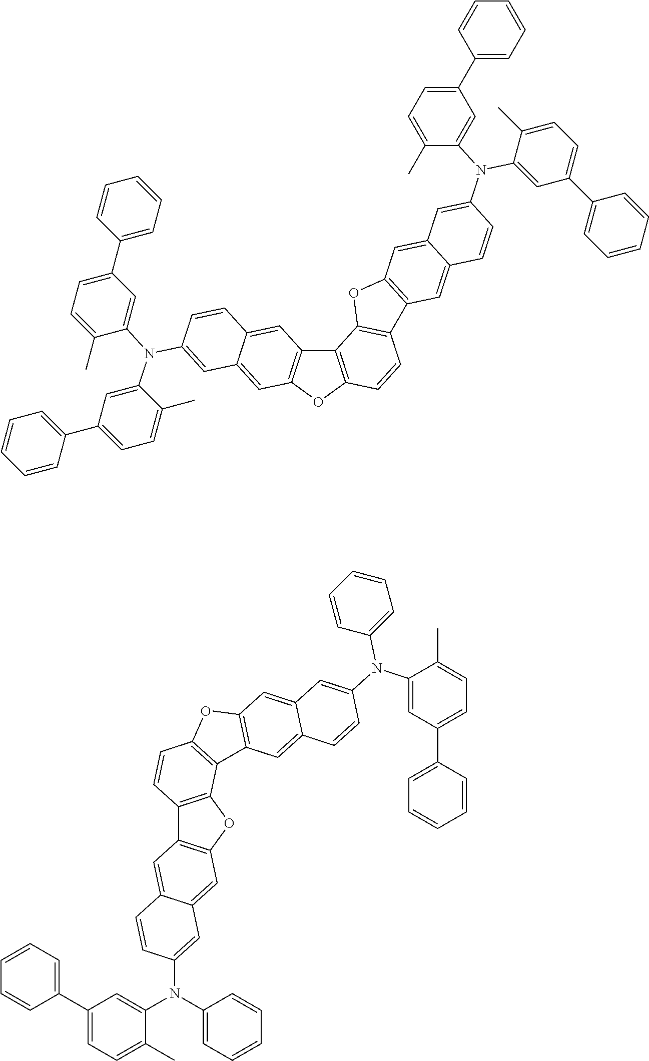 [SMILES: Cc1ccc(-c2ccccc2)cc1N(c1ccc2cc3c(cc2c1)oc1c3ccc2oc3cc4cc(N(c5cc(-c6ccccc6)ccc5C)c5cc(-c6ccccc6)ccc5C)ccc4cc3c21)c1cc(-c2ccccc2)ccc1C ; Cc1ccc(-c2ccccc2)cc1N(c1ccccc1)c1ccc2cc3c(cc2c1)oc1c3ccc2oc3cc4cc(N(c5ccccc5)c5cc(-c6ccccc6)ccc5C)ccc4cc3c21]

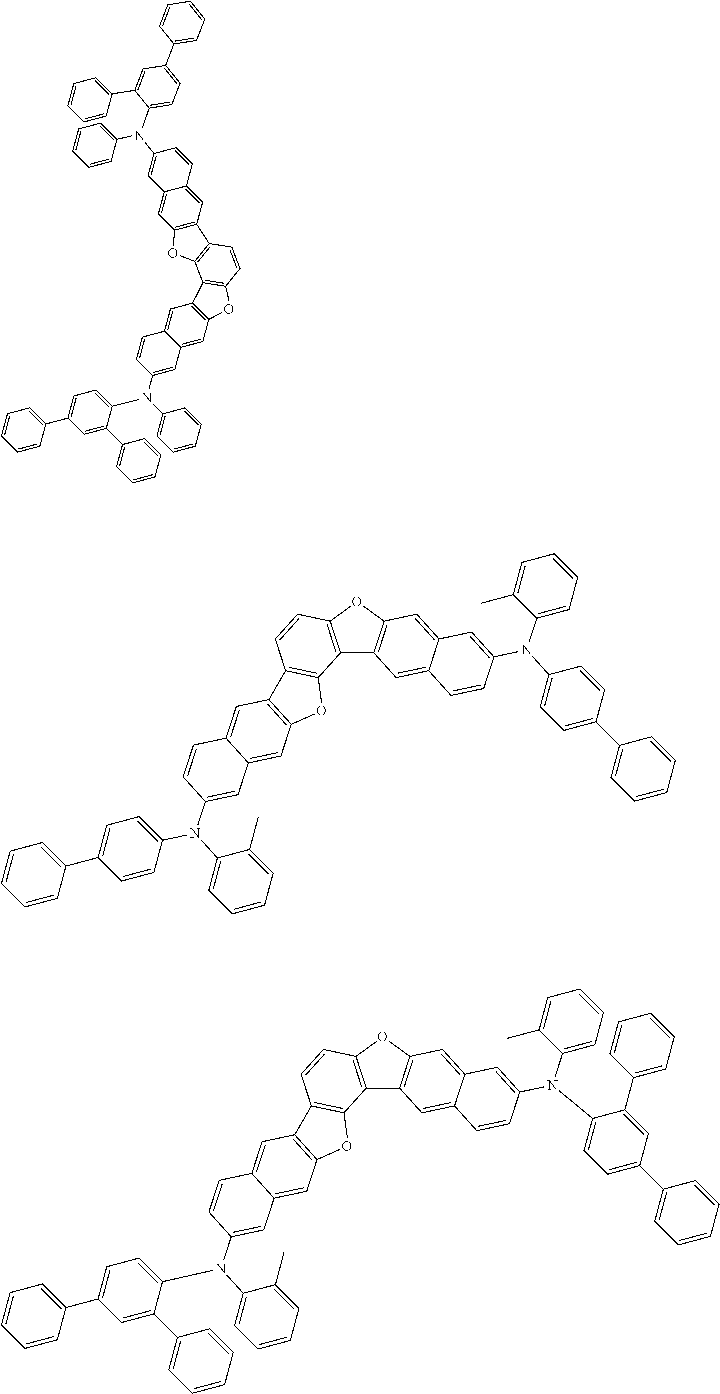 [SMILES: c1ccc(-c2ccc(N(c3ccccc3)c3ccc4cc5c(cc4c3)oc3c5ccc4oc5cc6cc(N(c7ccccc7)c7ccc(-c8ccccc8)cc7-c7ccccc7)ccc6cc5c43)c(-c3ccccc3)c2)cc1 ; Cc1ccccc1N(c1ccc(-c2ccccc2)cc1)c1ccc2cc3c(cc2c1)oc1c3ccc2oc3cc4cc(N(c5ccc(-c6ccccc6)cc5)c5ccccc5C)ccc4cc3c21 ; Cc1ccccc1N(c1ccc2cc3c(cc2c1)oc1c3ccc2oc3cc4cc(N(c5ccccc5C)c5ccc(-c6ccccc6)cc5-c5ccccc5)ccc4cc3c21)c1ccc(-c2ccccc2)cc1-c1ccccc1]

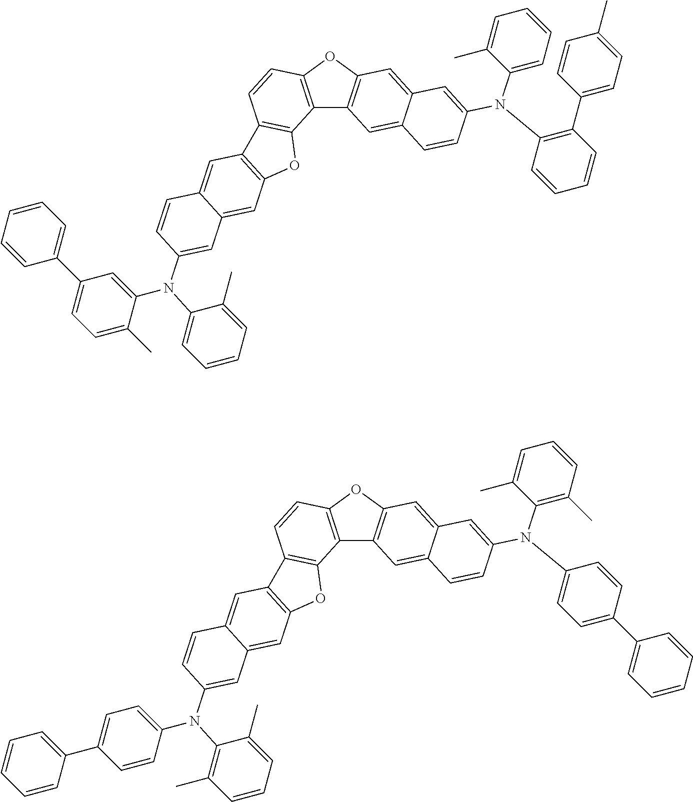 [SMILES: Cc1ccc(-c2ccccc2N(c2ccc3cc4c(cc3c2)oc2ccc3c5cc6ccc(N(c7ccccc7C)c7cc(-c8ccccc8)ccc7C)cc6cc5oc3c24)c2ccccc2C)cc1 ; Cc1cccc(C)c1N(c1ccc(-c2ccccc2)cc1)c1ccc2cc3c(cc2c1)oc1c3ccc2oc3cc4cc(N(c5ccc(-c6ccccc6)cc5)c5c(C)cccc5C)ccc4cc3c21]

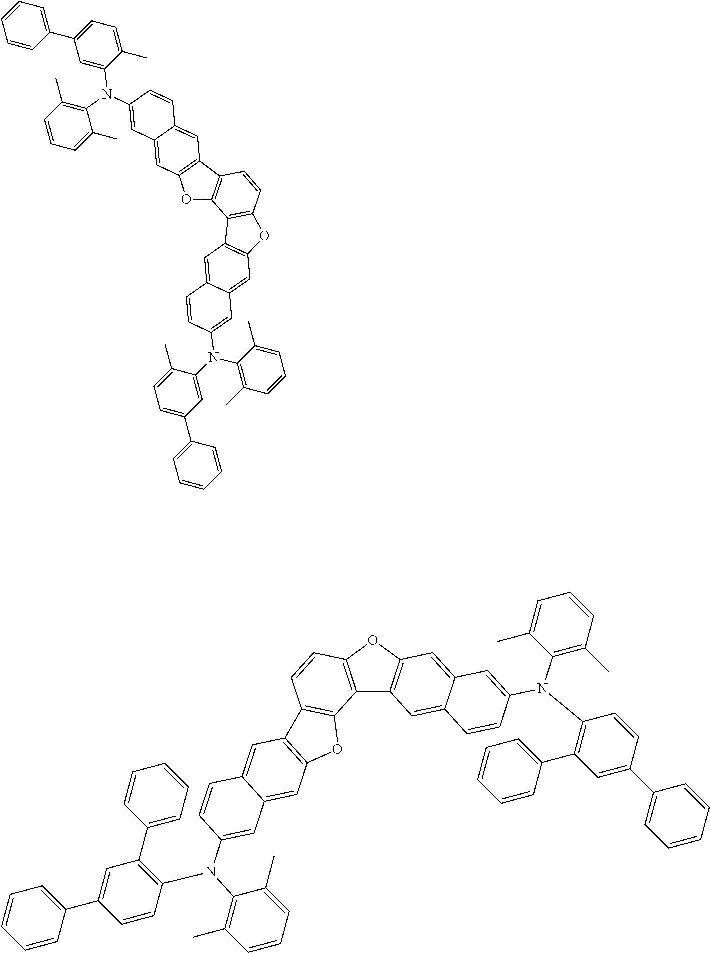 [SMILES: Cc1ccc(-c2ccccc2)cc1N(c1ccc2cc3c(cc2c1)oc1c3ccc2oc3cc4cc(N(c5cc(-c6ccccc6)ccc5C)c5c(C)cccc5C)ccc4cc3c21)c1c(C)cccc1C ; Cc1cccc(C)c1N(c1ccc2cc3c(cc2c1)oc1c3ccc2oc3cc4cc(N(c5ccc(-c6ccccc6)cc5-c5ccccc5)c5c(C)cccc5C)ccc4cc3c21)c1ccc(-c2ccccc2)cc1-c1ccccc1]

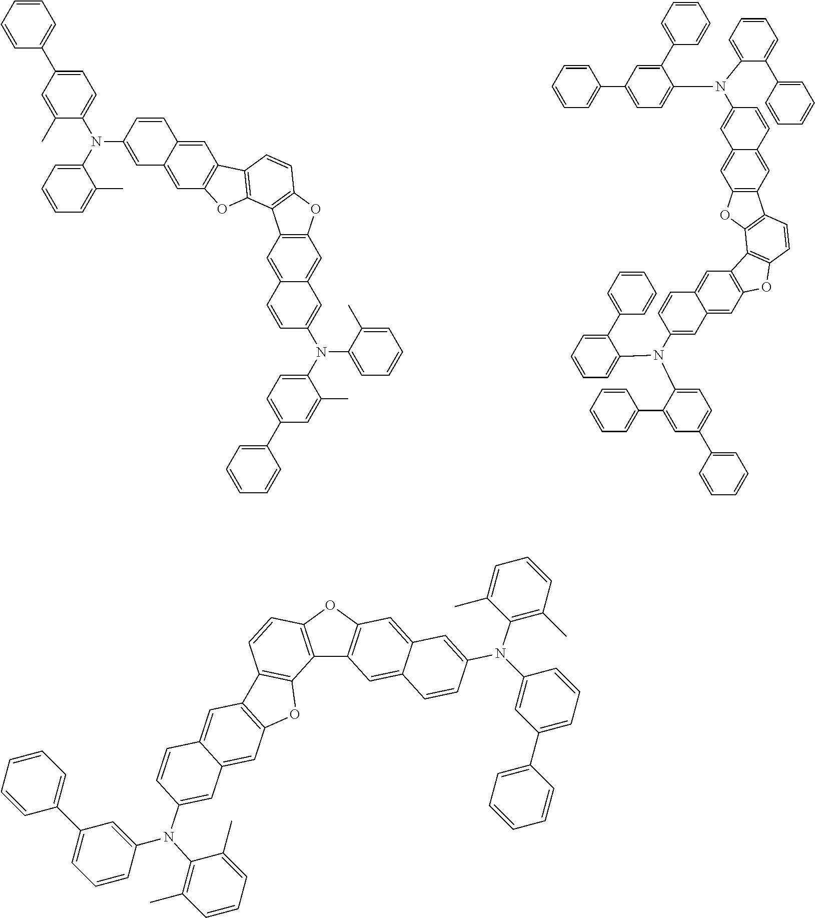 [SMILES: Cc1ccccc1N(c1ccc2cc3c(cc2c1)oc1c3ccc2oc3cc4cc(N(c5ccccc5C)c5ccc(-c6ccccc6)cc5C)ccc4cc3c21)c1ccc(-c2ccccc2)cc1C ; c1ccc(-c2ccc(N(c3ccc4cc5c(cc4c3)oc3c5ccc4oc5cc6cc(N(c7ccccc7-c7ccccc7)c7ccc(-c8ccccc8)cc7-c7ccccc7)ccc6cc5c43)c3ccccc3-c3ccccc3)c(-c3ccccc3)c2)cc1 ; Cc1cccc(C)c1N(c1cccc(-c2ccccc2)c1)c1ccc2cc3c(cc2c1)oc1c3ccc2oc3cc4cc(N(c5cccc(-c6ccccc6)c5)c5c(C)cccc5C)ccc4cc3c21]

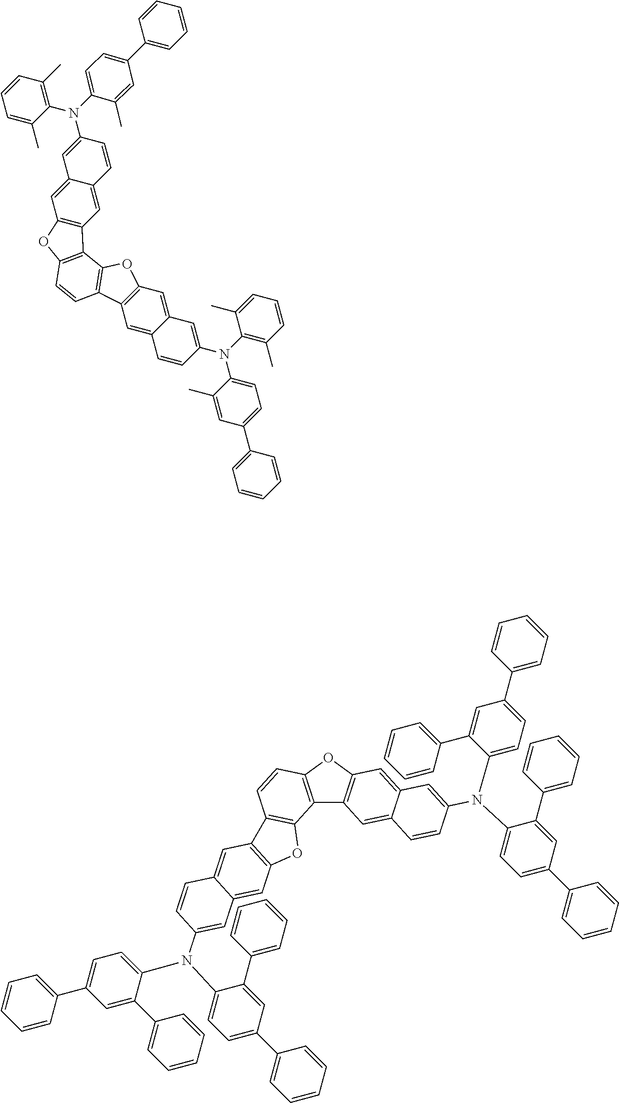 [SMILES: Cc1cc(-c2ccccc2)ccc1N(c1ccc2cc3c(cc2c1)oc1c3ccc2oc3cc4cc(N(c5ccc(-c6ccccc6)cc5C)c5c(C)cccc5C)ccc4cc3c21)c1c(C)cccc1C ; c1ccc(-c2ccc(N(c3ccc4cc5c(cc4c3)oc3c5ccc4oc5cc6cc(N(c7ccc(-c8ccccc8)cc7-c7ccccc7)c7ccc(-c8ccccc8)cc7-c7ccccc7)ccc6cc5c43)c3ccc(-c4ccccc4)cc3-c3ccccc3)c(-c3ccccc3)c2)cc1]

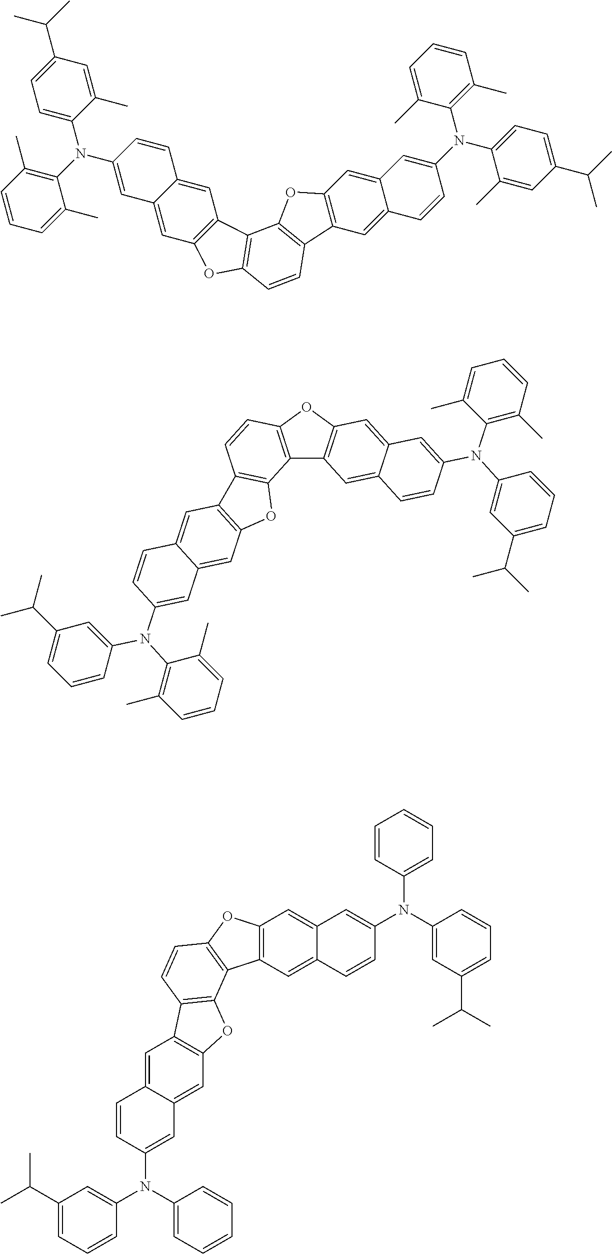

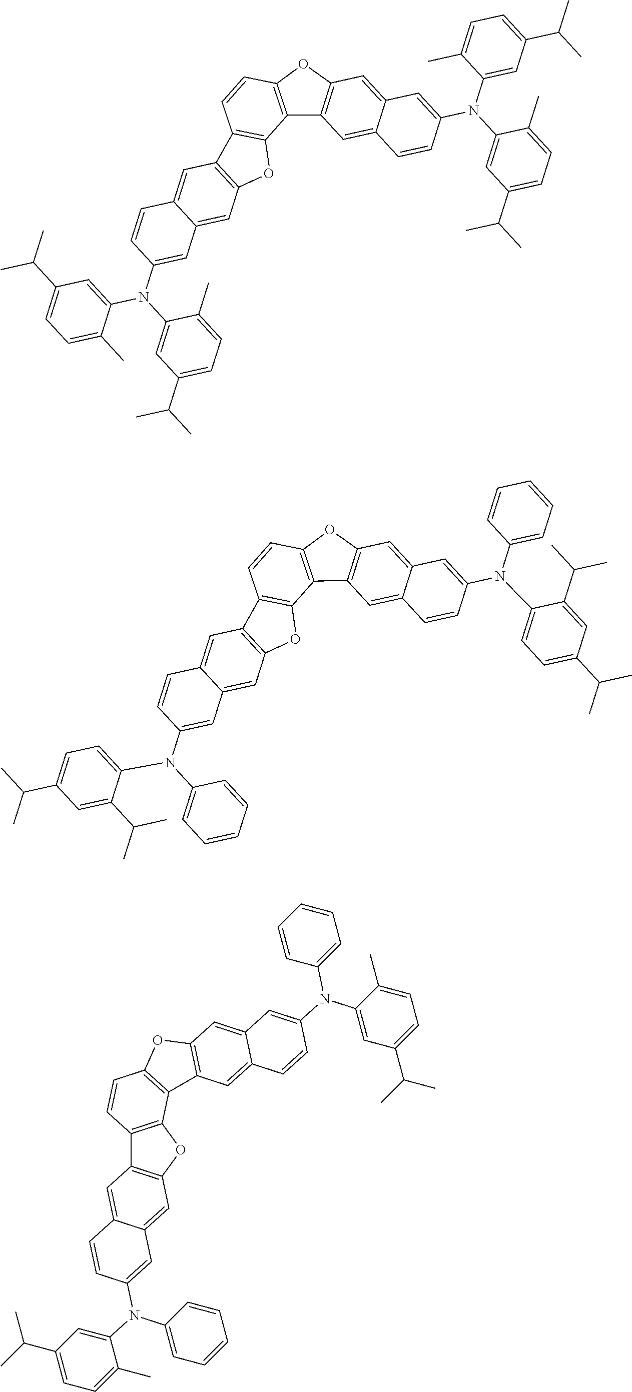

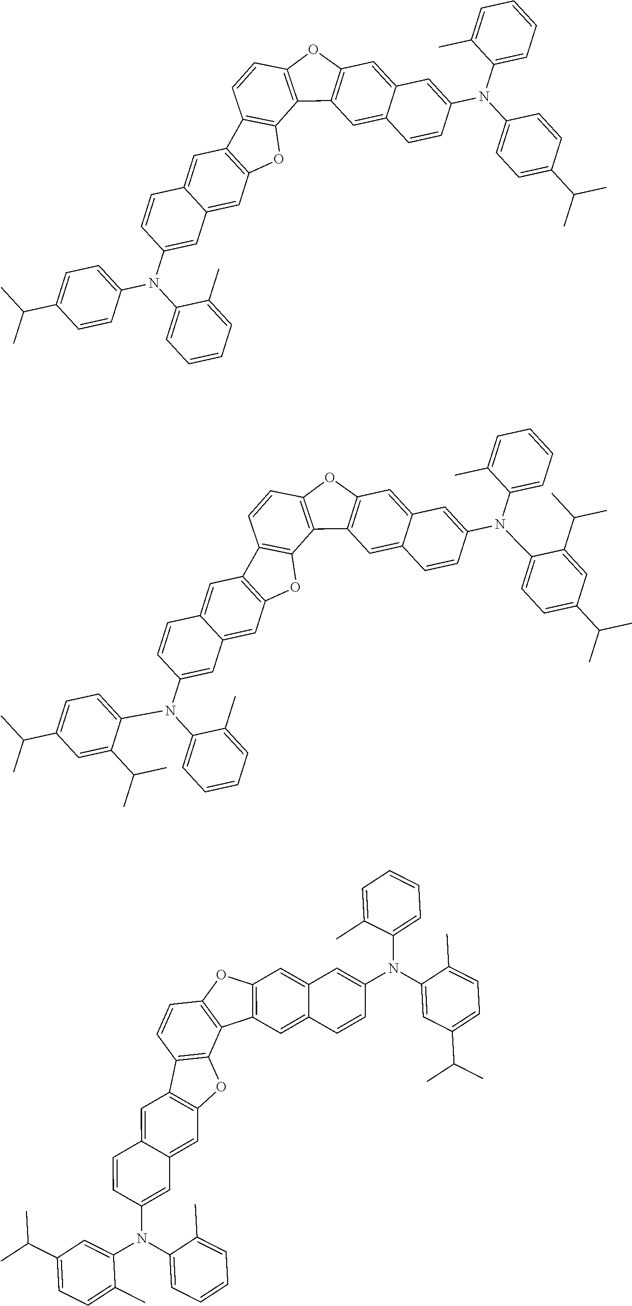

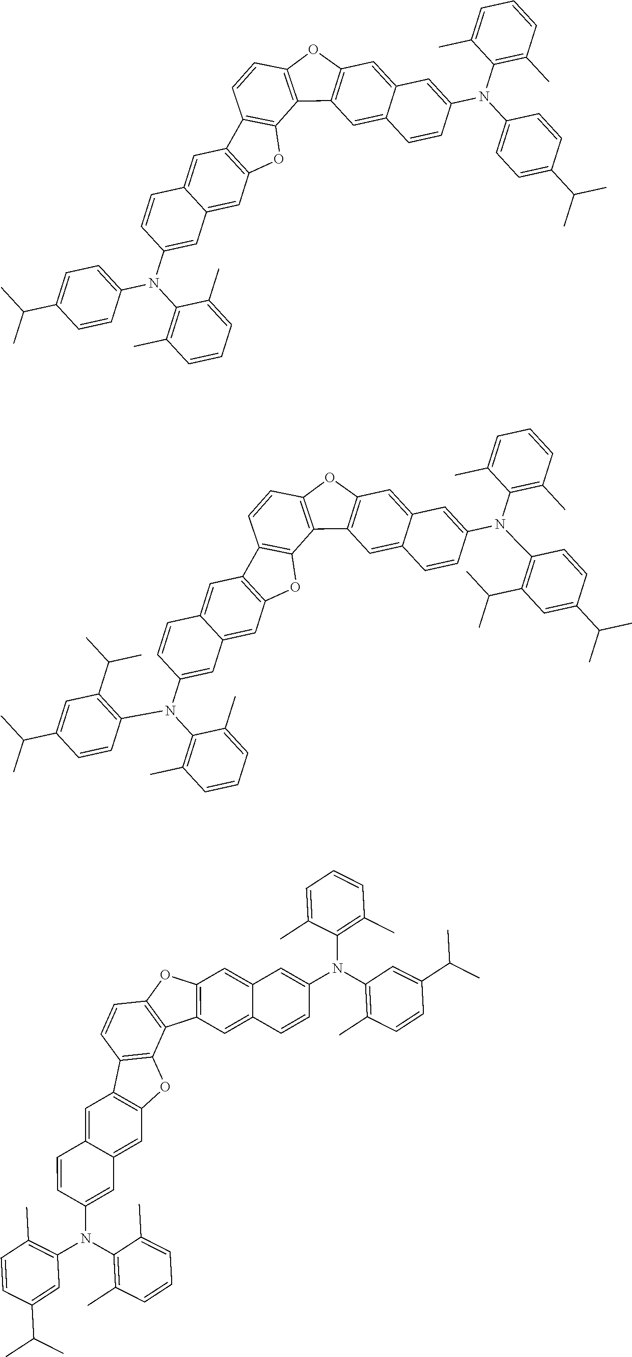

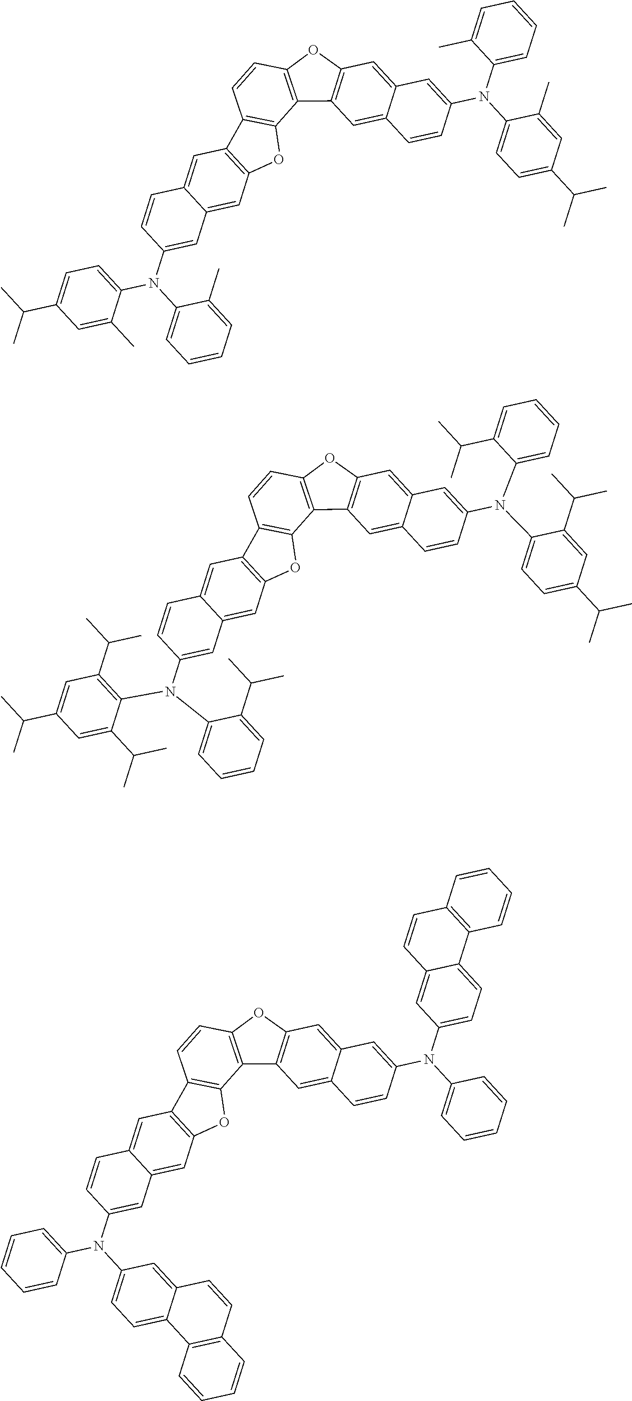

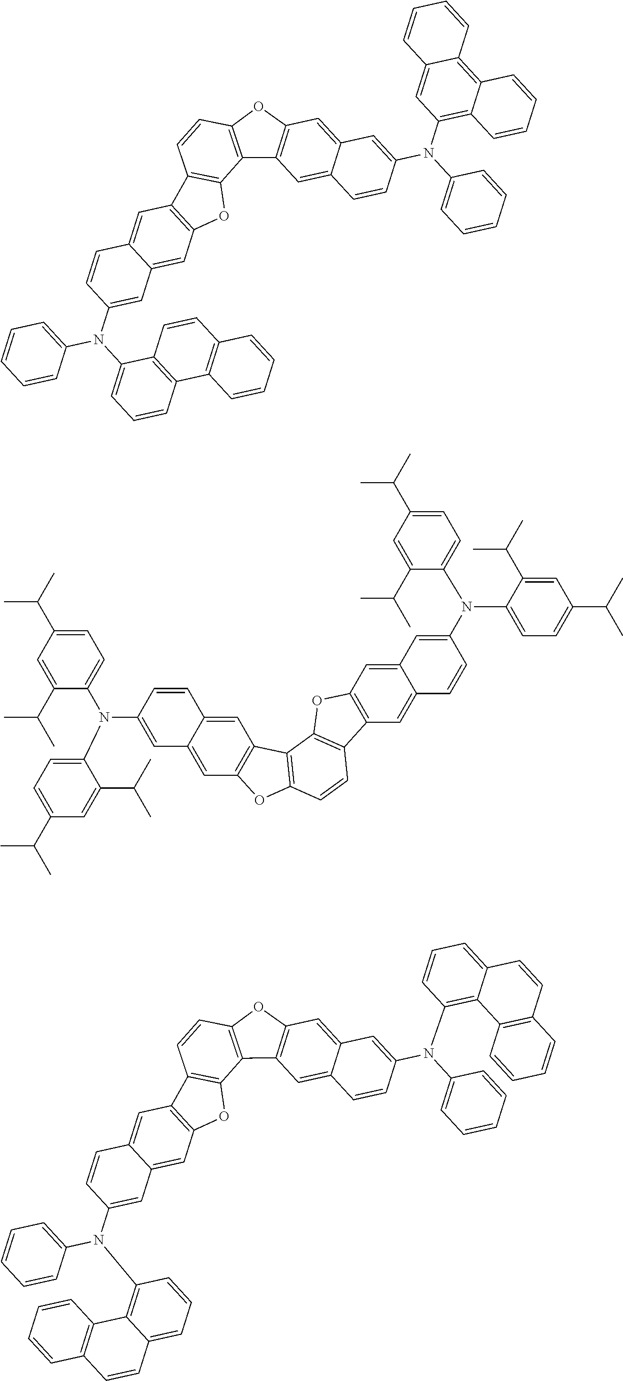 [SMILES: c1ccc(N(c2ccc3cc4c(cc3c2)oc2c4ccc3oc4cc5cc(N(c6ccccc6)c6cc7ccccc7c7ccccc67)ccc5cc4c32)c2cccc3c2ccc2ccccc23)cc1 ; CC(C)c1ccc(N(c2ccc3cc4c(cc3c2)oc2c4ccc3oc4cc5cc(N(c6ccc(C(C)C)cc6C(C)C)c6ccc(C(C)C)cc6C(C)C)ccc5cc4c32)c2ccc(C(C)C)cc2C(C)C)c(C(C)C)c1 ; c1ccc(N(c2ccc3cc4c(cc3c2)oc2c4ccc3oc4cc5cc(N(c6ccccc6)c6cccc7ccc8ccccc8c67)ccc5cc4c32)c2cccc3ccc4ccccc4c23)cc1]

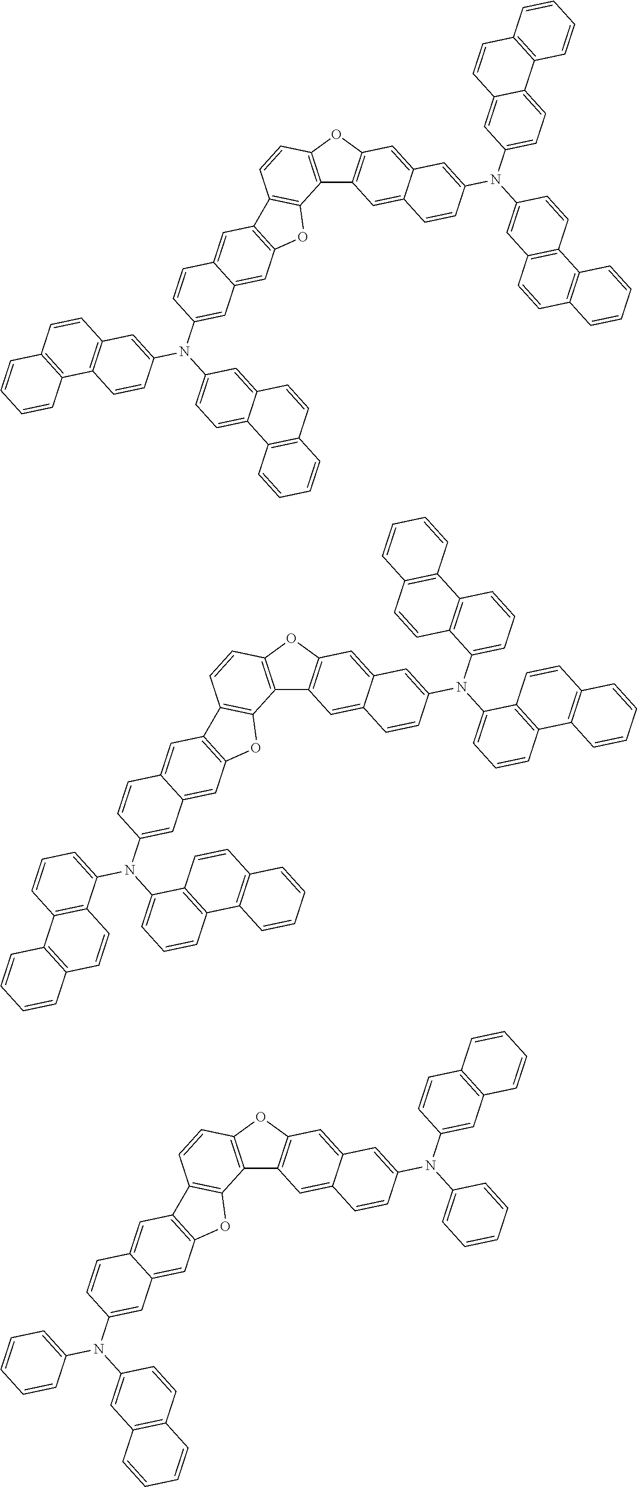 [SMILES: c1ccc2c(c1)ccc1cc(N(c3ccc4cc5c(cc4c3)oc3c5ccc4oc5cc6cc(N(c7ccc8c(ccc9ccccc98)c7)c7ccc8c(ccc9ccccc98)c7)ccc6cc5c43)c3ccc4c(ccc5ccccc54)c3)ccc12 ; c1ccc2c(c1)ccc1c(N(c3ccc4cc5c(cc4c3)oc3c5ccc4oc5cc6cc(N(c7cccc8c7ccc7ccccc78)c7cccc8c7ccc7ccccc78)ccc6cc5c43)c3cccc4c3ccc3ccccc34)cccc12 ; c1ccc(N(c2ccc3ccccc3c2)c2ccc3cc4c(cc3c2)oc2c4ccc3oc4cc5cc(N(c6ccccc6)c6ccc7ccccc7c6)ccc5cc4c32)cc1]

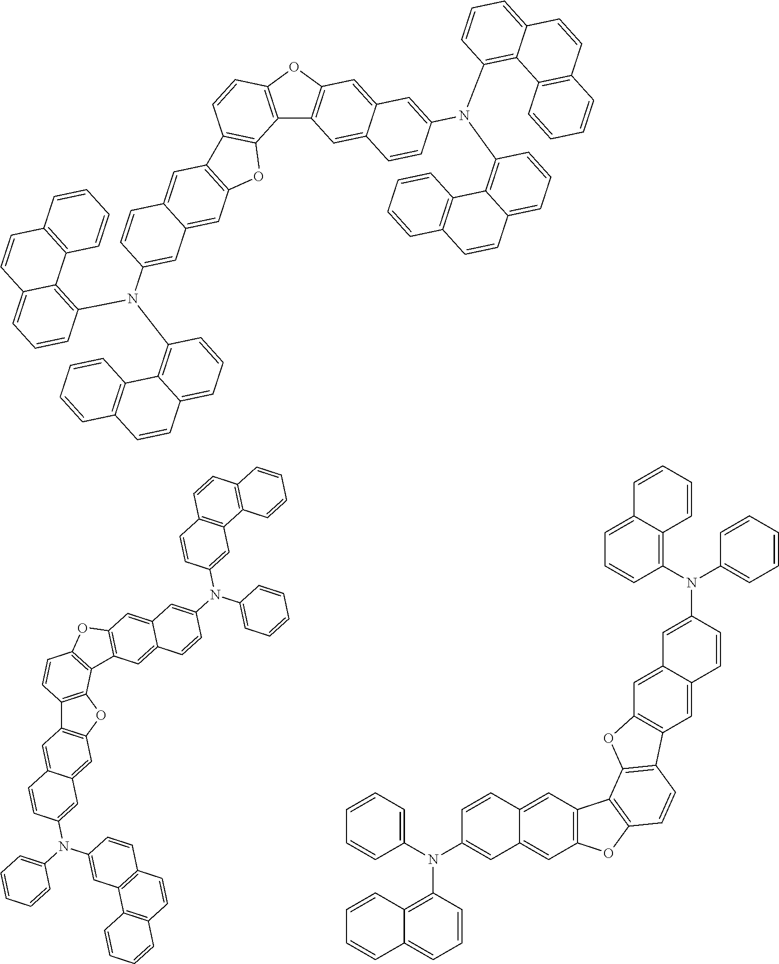 [SMILES: c1ccc2c(c1)ccc1cccc(N(c3ccc4cc5c(cc4c3)oc3c5ccc4oc5cc6cc(N(c7cccc8ccc9ccccc9c78)c7cccc8ccc9ccccc9c78)ccc6cc5c43)c3cccc4ccc5ccccc5c34)c12 ; c1ccc(N(c2ccc3cc4c(cc3c2)oc2c4ccc3oc4cc5cc(N(c6ccccc6)c6ccc7ccc8ccccc8c7c6)ccc5cc4c32)c2ccc3ccc4ccccc4c3c2)cc1 ; c1ccc(N(c2ccc3cc4c(cc3c2)oc2c4ccc3oc4cc5cc(N(c6ccccc6)c6cccc7ccccc67)ccc5cc4c32)c2cccc3ccccc23)cc1]

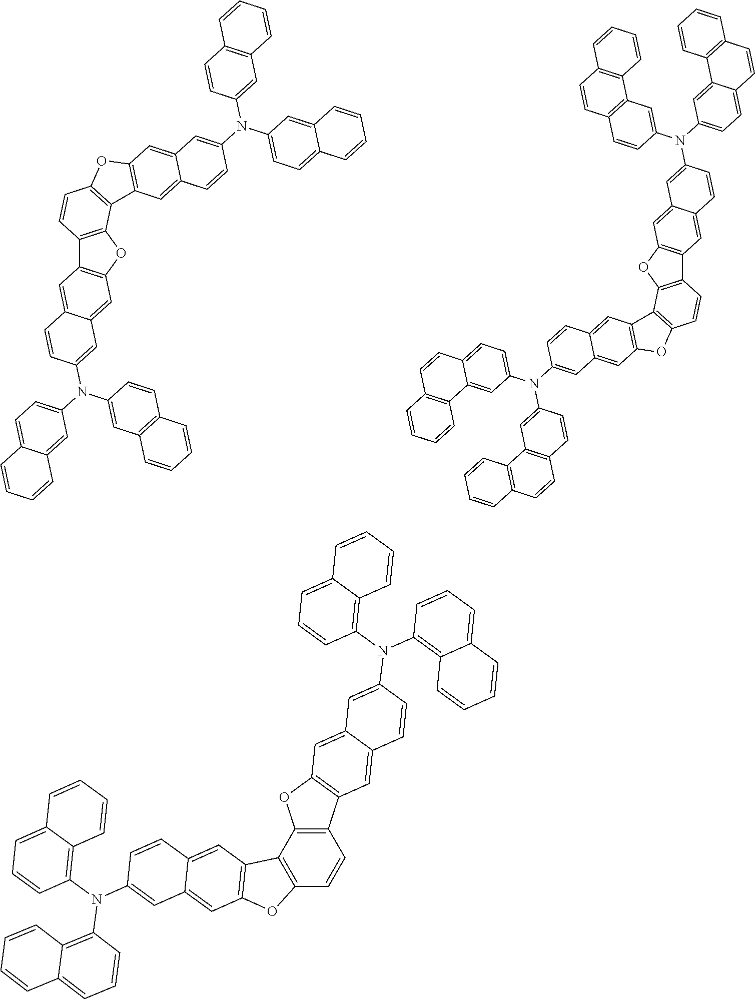 [SMILES: c1ccc2cc(N(c3ccc4ccccc4c3)c3ccc4cc5c(cc4c3)oc3c5ccc4oc5cc6cc(N(c7ccc8ccccc8c7)c7ccc8ccccc8c7)ccc6cc5c43)ccc2c1 ; c1ccc2c(c1)ccc1ccc(N(c3ccc4cc5c(cc4c3)oc3c5ccc4oc5cc6cc(N(c7ccc8ccc9ccccc9c8c7)c7ccc8ccc9ccccc9c8c7)ccc6cc5c43)c3ccc4ccc5ccccc5c4c3)cc12 ; c1ccc2c(N(c3ccc4cc5c(cc4c3)oc3c5ccc4oc5cc6cc(N(c7cccc8ccccc78)c7cccc8ccccc78)ccc6cc5c43)c3cccc4ccccc34)cccc2c1]

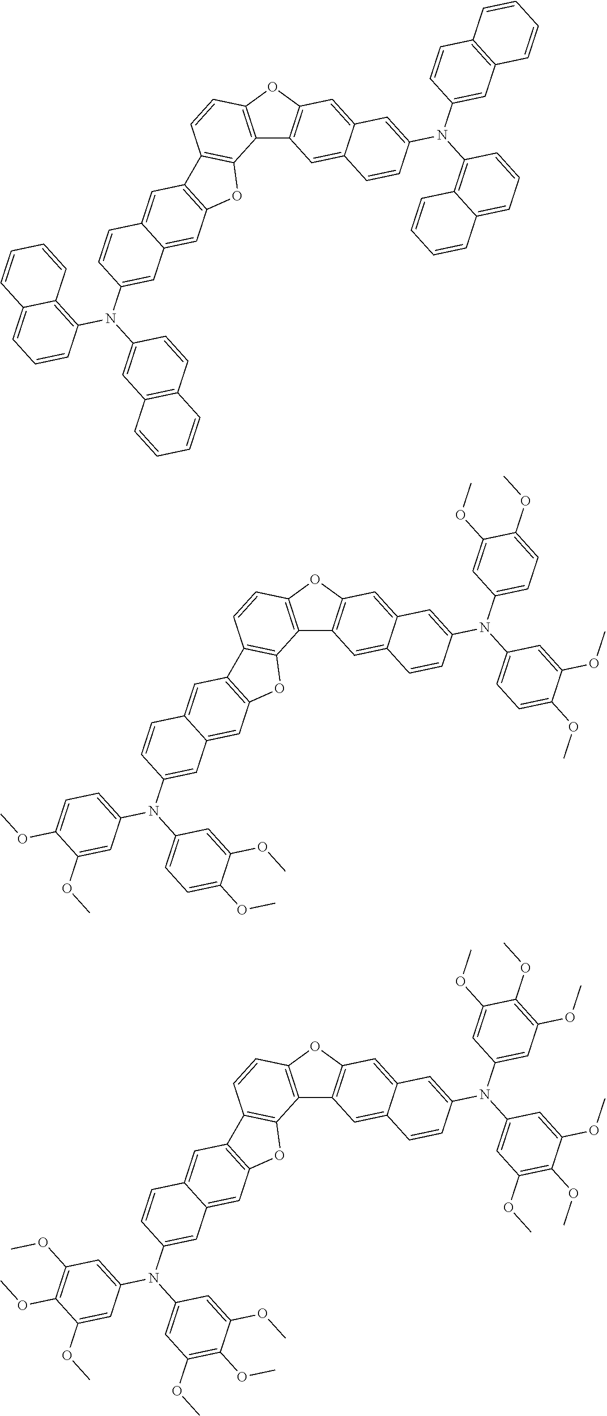

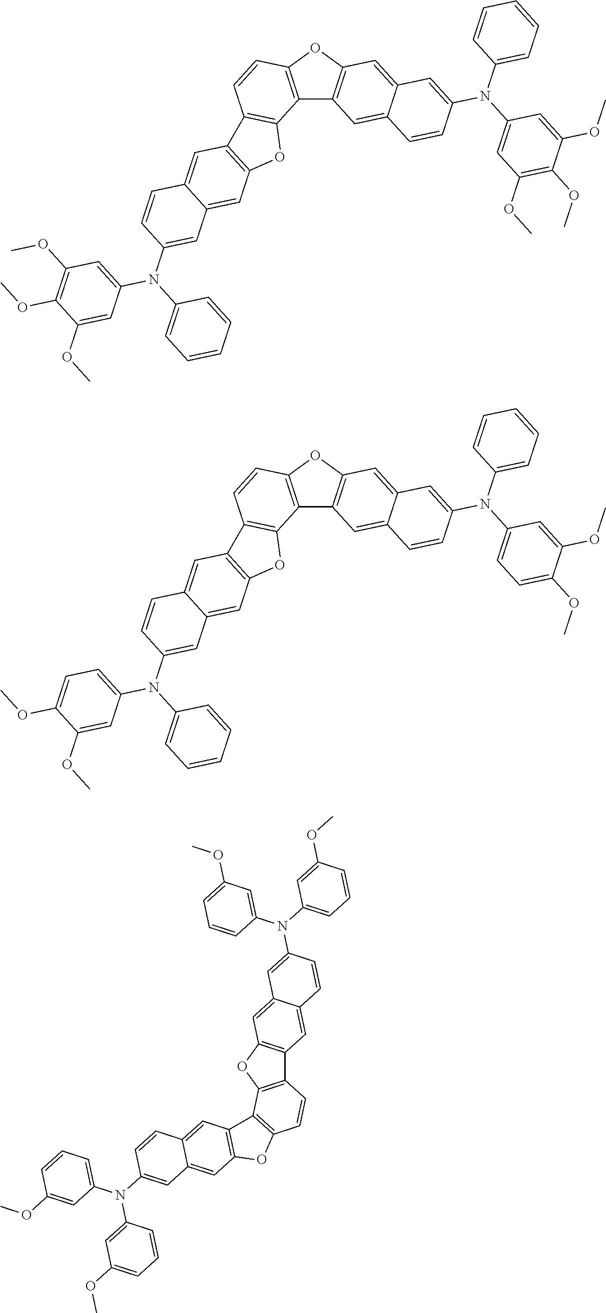

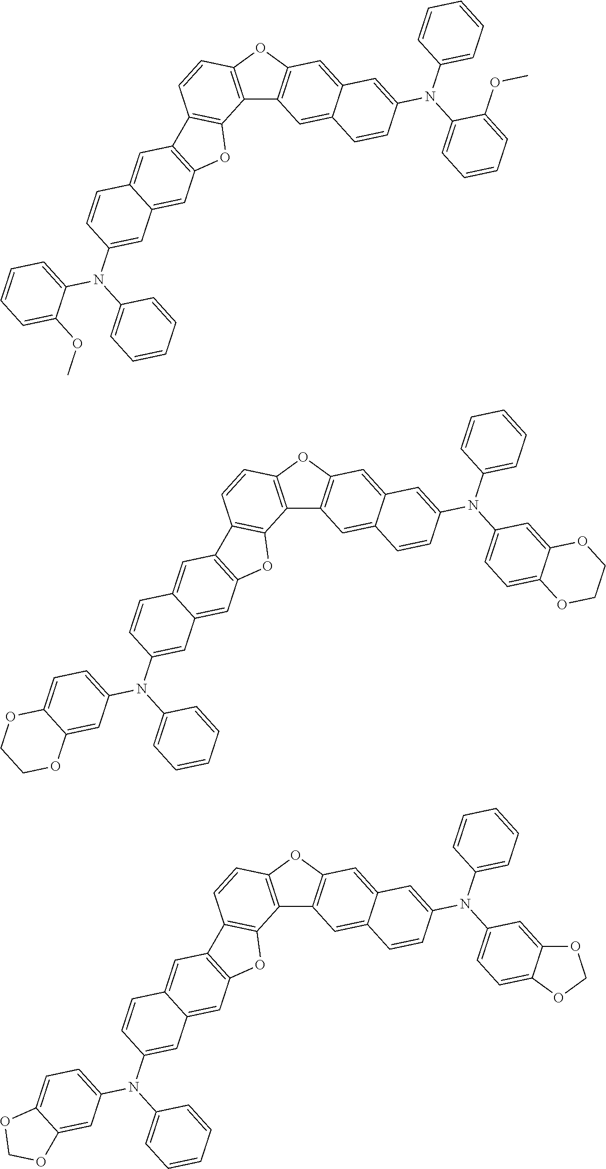 [SMILES: COc1ccccc1N(c1ccccc1)c1ccc2cc3c(cc2c1)oc1c3ccc2oc3cc4cc(N(c5ccccc5)c5ccccc5OC)ccc4cc3c21 ; c1ccc(N(c2ccc3c(c2)OCCO3)c2ccc3cc4c(cc3c2)oc2c4ccc3oc4cc5cc(N(c6ccccc6)c6ccc7c(c6)OCCO7)ccc5cc4c32)cc1 ; c1ccc(N(c2ccc3c(c2)OCO3)c2ccc3cc4c(cc3c2)oc2c4ccc3oc4cc5cc(N(c6ccccc6)c6ccc7c(c6)OCO7)ccc5cc4c32)cc1]

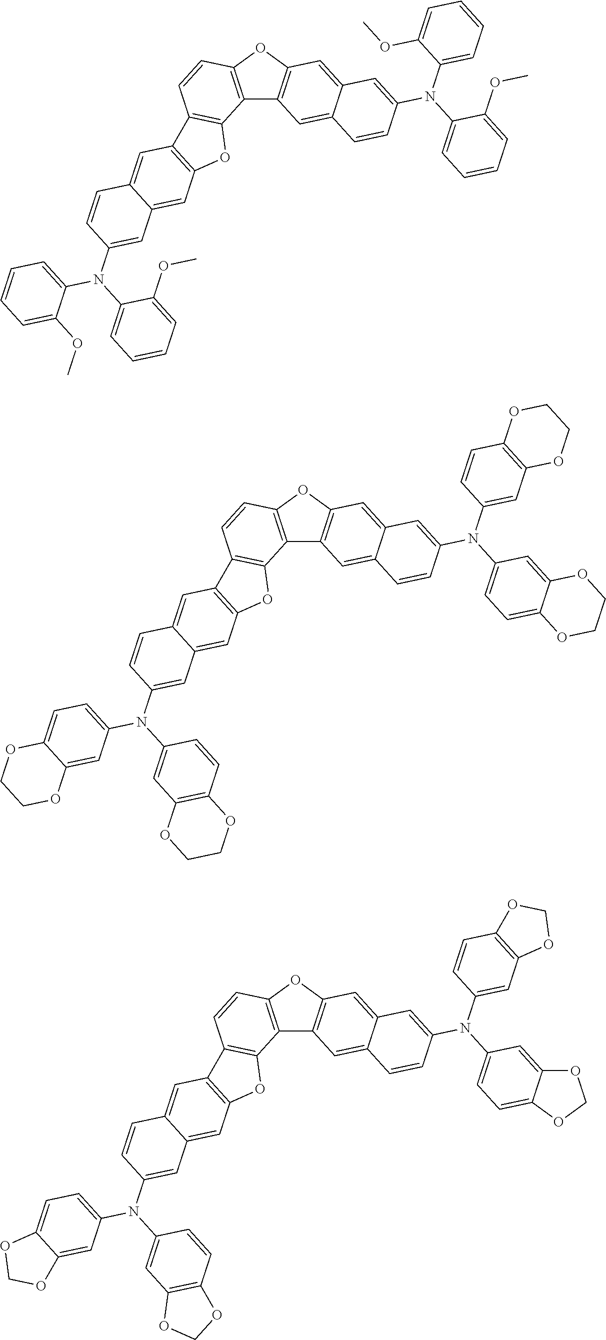 [SMILES: COc1ccccc1N(c1ccc2cc3c(cc2c1)oc1c3ccc2oc3cc4cc(N(c5ccccc5OC)c5ccccc5OC)ccc4cc3c21)c1ccccc1OC ; c1cc2c(cc1N(c1ccc3c(c1)OCCO3)c1ccc3cc4c(cc3c1)oc1c4ccc3oc4cc5cc(N(c6ccc7c(c6)OCCO7)c6ccc7c(c6)OCCO7)ccc5cc4c31)OCCO2 ; c1cc2c(cc1N(c1ccc3c(c1)OCO3)c1ccc3cc4c(cc3c1)oc1c4ccc3oc4cc5cc(N(c6ccc7c(c6)OCO7)c6ccc7c(c6)OCO7)ccc5cc4c31)OCO2]

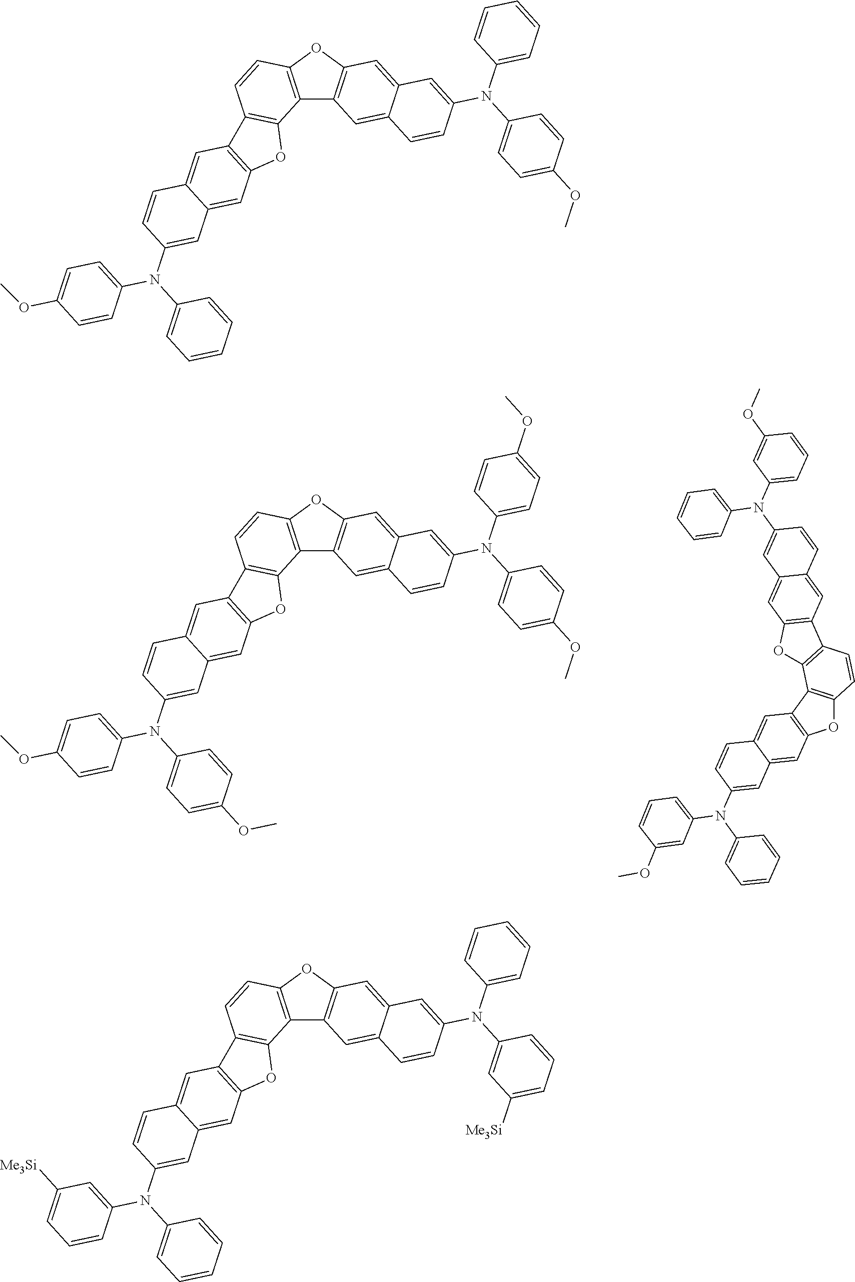 [SMILES: COc1ccc(N(c2ccccc2)c2ccc3cc4c(cc3c2)oc2c4ccc3oc4cc5cc(N(c6ccccc6)c6ccc(OC)cc6)ccc5cc4c32)cc1 ; COc1ccc(N(c2ccc(OC)cc2)c2ccc3cc4c(cc3c2)oc2c4ccc3oc4cc5cc(N(c6ccc(OC)cc6)c6ccc(OC)cc6)ccc5cc4c32)cc1 ; COc1cccc(N(c2ccccc2)c2ccc3cc4c(cc3c2)oc2c4ccc3oc4cc5cc(N(c6ccccc6)c6cccc(OC)c6)ccc5cc4c32)c1 ; C[Si](C)(C)c1cccc(N(c2ccccc2)c2ccc3cc4c(cc3c2)oc2c4ccc3oc4cc5cc(N(c6ccccc6)c6cccc([Si](C)(C)C)c6)ccc5cc4c32)c1]

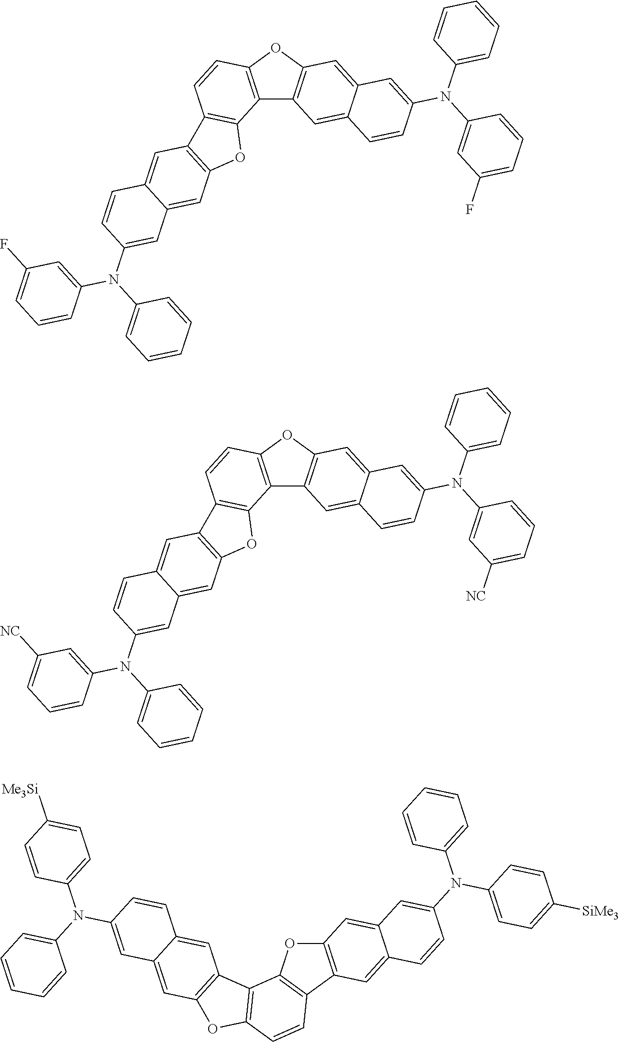 [SMILES: Fc1cccc(N(c2ccccc2)c2ccc3cc4c(cc3c2)oc2c4ccc3oc4cc5cc(N(c6ccccc6)c6cccc(F)c6)ccc5cc4c32)c1 ; N#Cc1cccc(N(c2ccccc2)c2ccc3cc4c(cc3c2)oc2c4ccc3oc4cc5cc(N(c6ccccc6)c6cccc(C#N)c6)ccc5cc4c32)c1 ; C[Si](C)(C)c1ccc(N(c2ccccc2)c2ccc3cc4c(cc3c2)oc2c4ccc3oc4cc5cc(N(c6ccccc6)c6ccc([Si](C)(C)C)cc6)ccc5cc4c32)cc1]

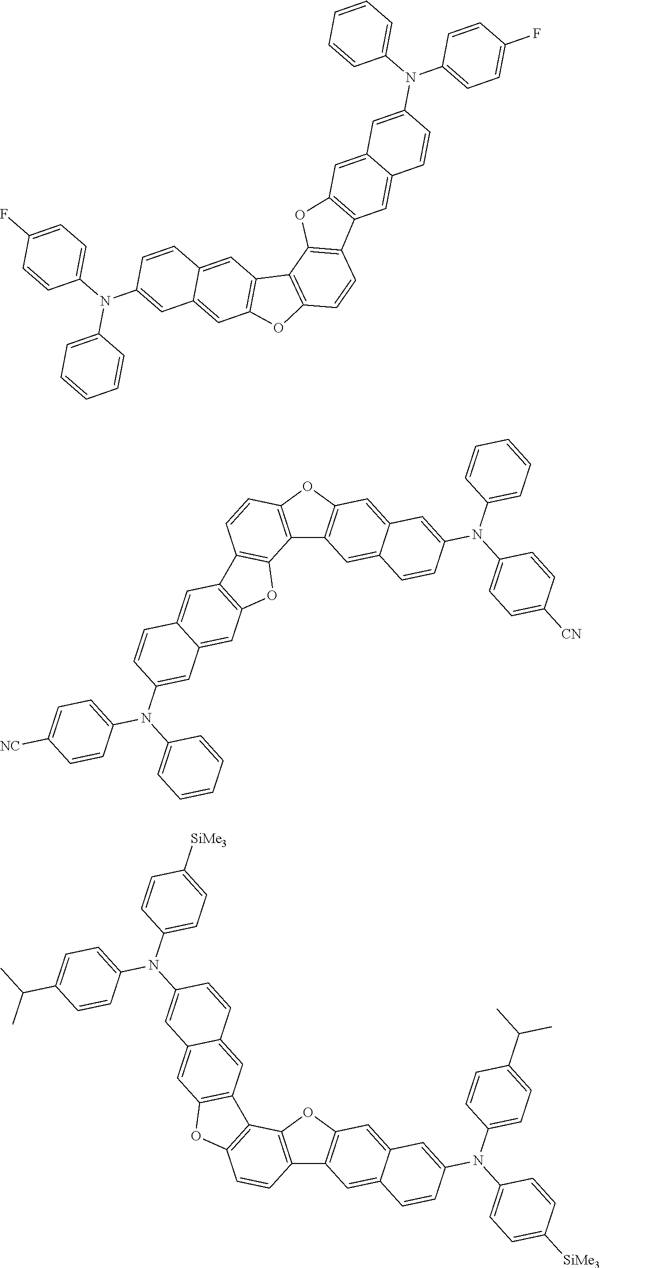

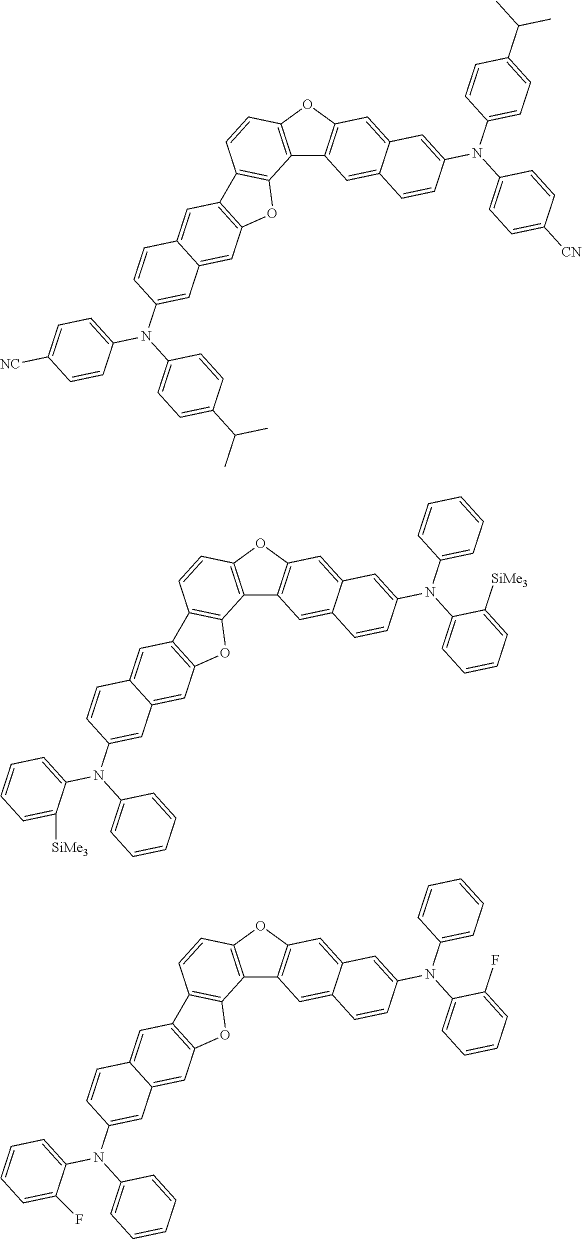 [SMILES: CC(C)c1ccc(N(c2ccc(C#N)cc2)c2ccc3cc4c(cc3c2)oc2c4ccc3oc4cc5cc(N(c6ccc(C#N)cc6)c6ccc(C(C)C)cc6)ccc5cc4c32)cc1 ; C[Si](C)(C)c1ccccc1N(c1ccccc1)c1ccc2cc3c(cc2c1)oc1c3ccc2oc3cc4cc(N(c5ccccc5)c5ccccc5[Si](C)(C)C)ccc4cc3c21 ; Fc1ccccc1N(c1ccccc1)c1ccc2cc3c(cc2c1)oc1c3ccc2oc3cc4cc(N(c5ccccc5)c5ccccc5F)ccc4cc3c21]

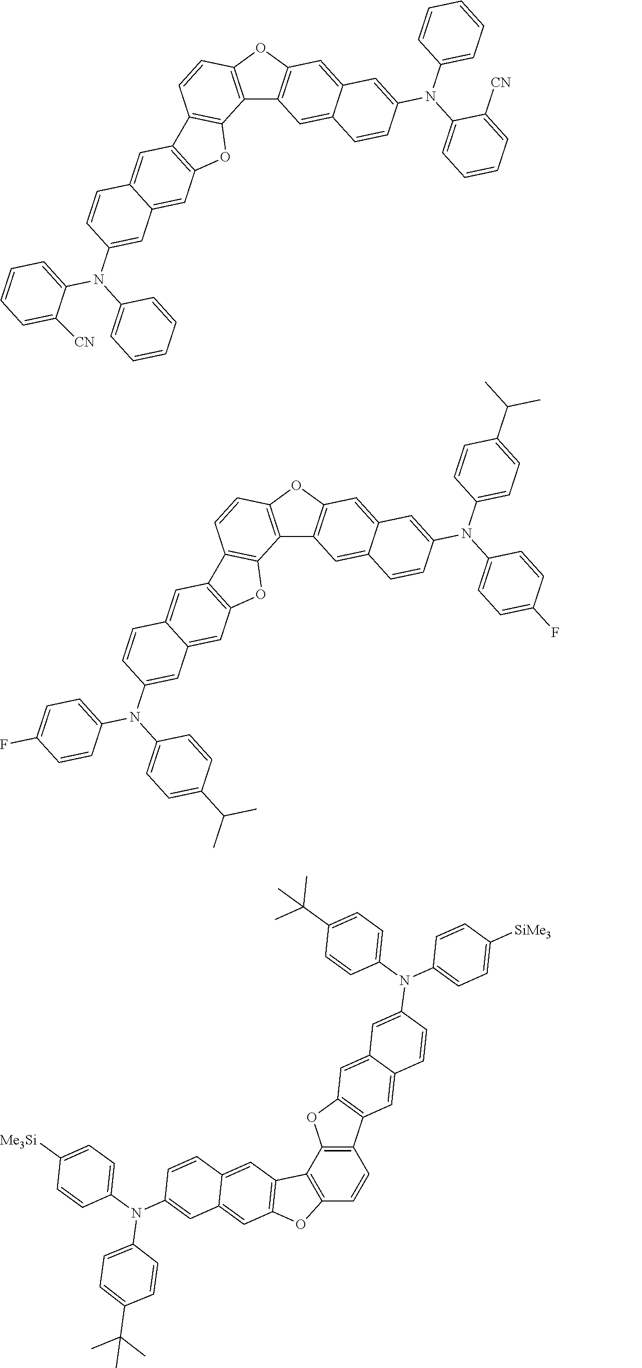 [SMILES: N#Cc1ccccc1N(c1ccccc1)c1ccc2cc3c(cc2c1)oc1c3ccc2oc3cc4cc(N(c5ccccc5)c5ccccc5C#N)ccc4cc3c21 ; CC(C)c1ccc(N(c2ccc(F)cc2)c2ccc3cc4c(cc3c2)oc2c4ccc3oc4cc5cc(N(c6ccc(F)cc6)c6ccc(C(C)C)cc6)ccc5cc4c32)cc1 ; CC(C)(C)c1ccc(N(c2ccc([Si](C)(C)C)cc2)c2ccc3cc4c(cc3c2)oc2c4ccc3oc4cc5cc(N(c6ccc(C(C)(C)C)cc6)c6ccc([Si](C)(C)C)cc6)ccc5cc4c32)cc1]

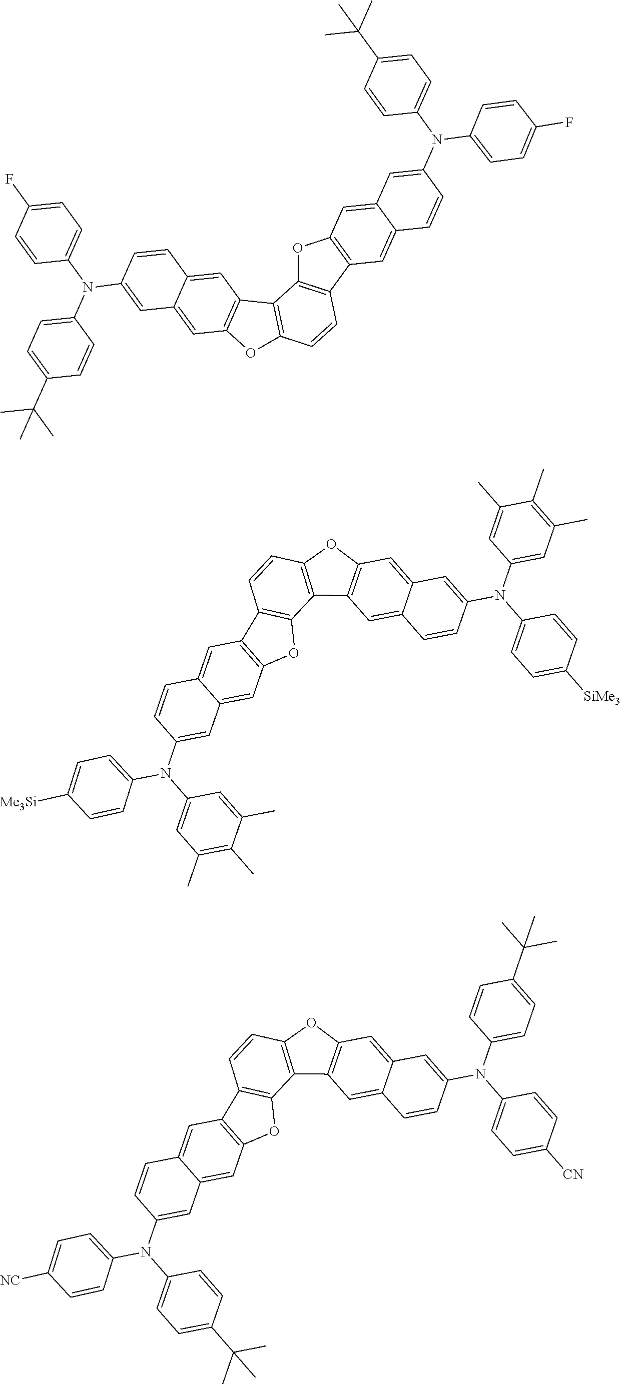 [SMILES: CC(C)(C)c1ccc(N(c2ccc(F)cc2)c2ccc3cc4c(cc3c2)oc2c4ccc3oc4cc5cc(N(c6ccc(F)cc6)c6ccc(C(C)(C)C)cc6)ccc5cc4c32)cc1 ; Cc1cc(N(c2ccc([Si](C)(C)C)cc2)c2ccc3cc4c(cc3c2)oc2c4ccc3oc4cc5cc(N(c6ccc([Si](C)(C)C)cc6)c6cc(C)c(C)c(C)c6)ccc5cc4c32)cc(C)c1C ; CC(C)(C)c1ccc(N(c2ccc(C#N)cc2)c2ccc3cc4c(cc3c2)oc2c4ccc3oc4cc5cc(N(c6ccc(C#N)cc6)c6ccc(C(C)(C)C)cc6)ccc5cc4c32)cc1]

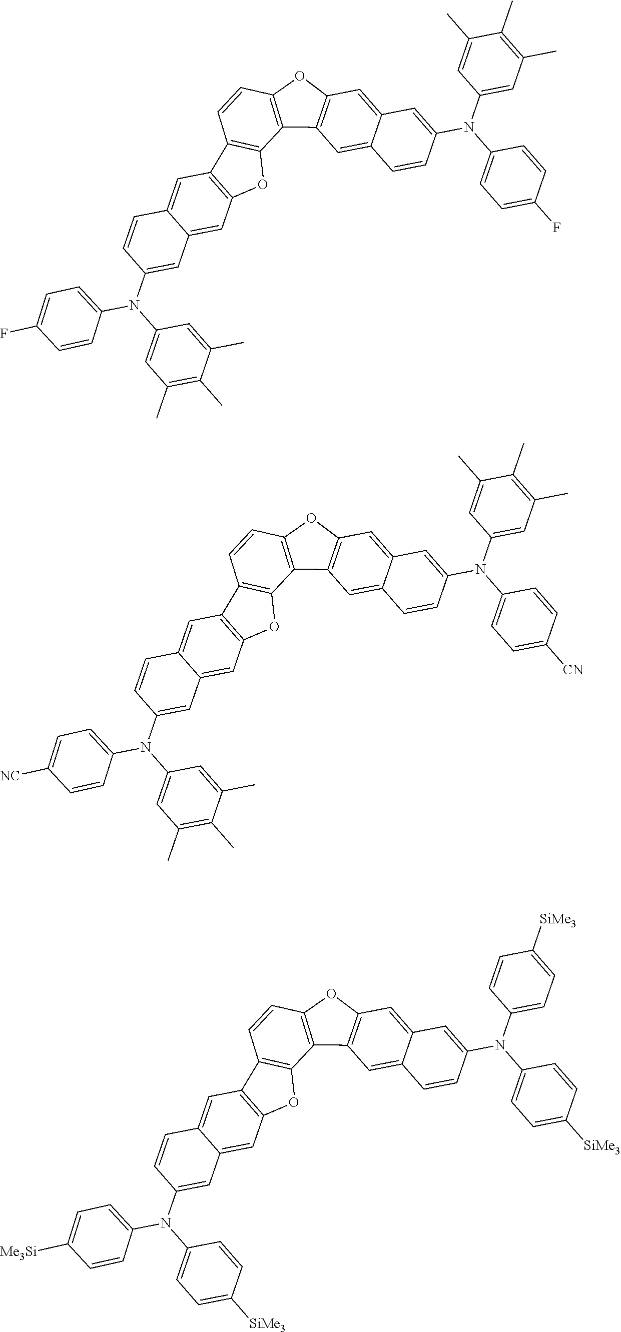 [SMILES: Cc1cc(N(c2ccc(F)cc2)c2ccc3cc4c(cc3c2)oc2c4ccc3oc4cc5cc(N(c6ccc(F)cc6)c6cc(C)c(C)c(C)c6)ccc5cc4c32)cc(C)c1C ; Cc1cc(N(c2ccc(C#N)cc2)c2ccc3cc4c(cc3c2)oc2c4ccc3oc4cc5cc(N(c6ccc(C#N)cc6)c6cc(C)c(C)c(C)c6)ccc5cc4c32)cc(C)c1C ; C[Si](C)(C)c1ccc(N(c2ccc([Si](C)(C)C)cc2)c2ccc3cc4c(cc3c2)oc2c4ccc3oc4cc5cc(N(c6ccc([Si](C)(C)C)cc6)c6ccc([Si](C)(C)C)cc6)ccc5cc4c32)cc1]

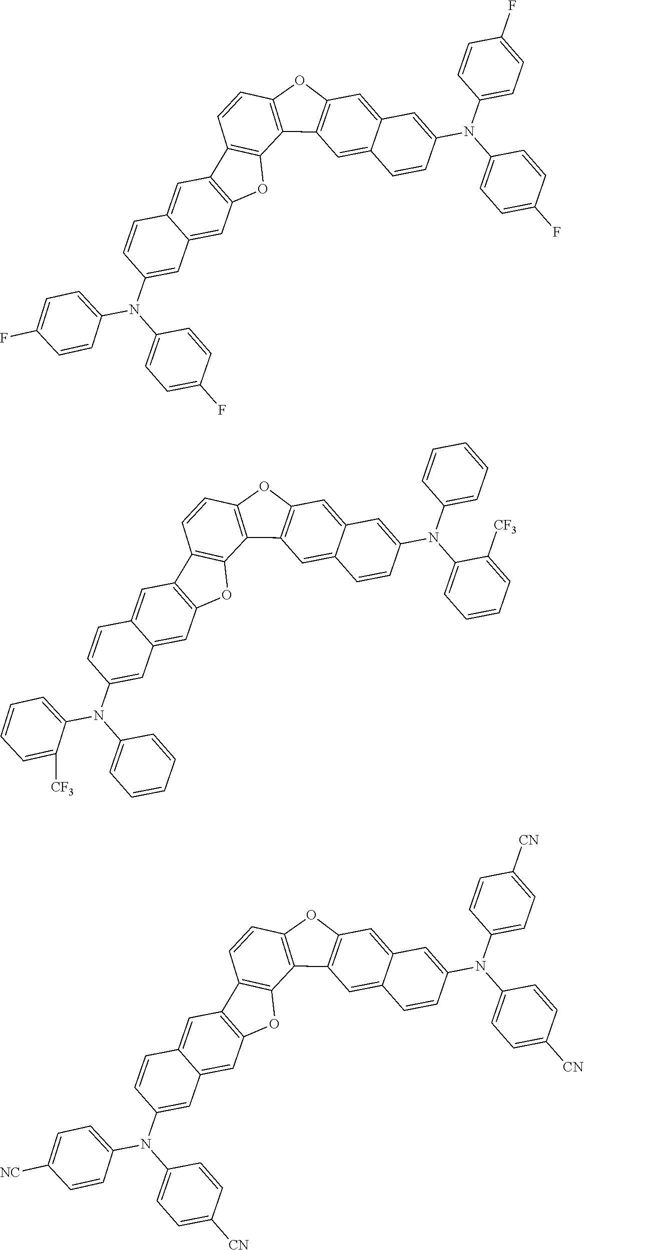 [SMILES: Fc1ccc(N(c2ccc(F)cc2)c2ccc3cc4c(cc3c2)oc2c4ccc3oc4cc5cc(N(c6ccc(F)cc6)c6ccc(F)cc6)ccc5cc4c32)cc1 ; FC(F)(F)c1ccccc1N(c1ccccc1)c1ccc2cc3c(cc2c1)oc1c3ccc2oc3cc4cc(N(c5ccccc5)c5ccccc5C(F)(F)F)ccc4cc3c21 ; N#Cc1ccc(N(c2ccc(C#N)cc2)c2ccc3cc4c(cc3c2)oc2c4ccc3oc4cc5cc(N(c6ccc(C#N)cc6)c6ccc(C#N)cc6)ccc5cc4c32)cc1]

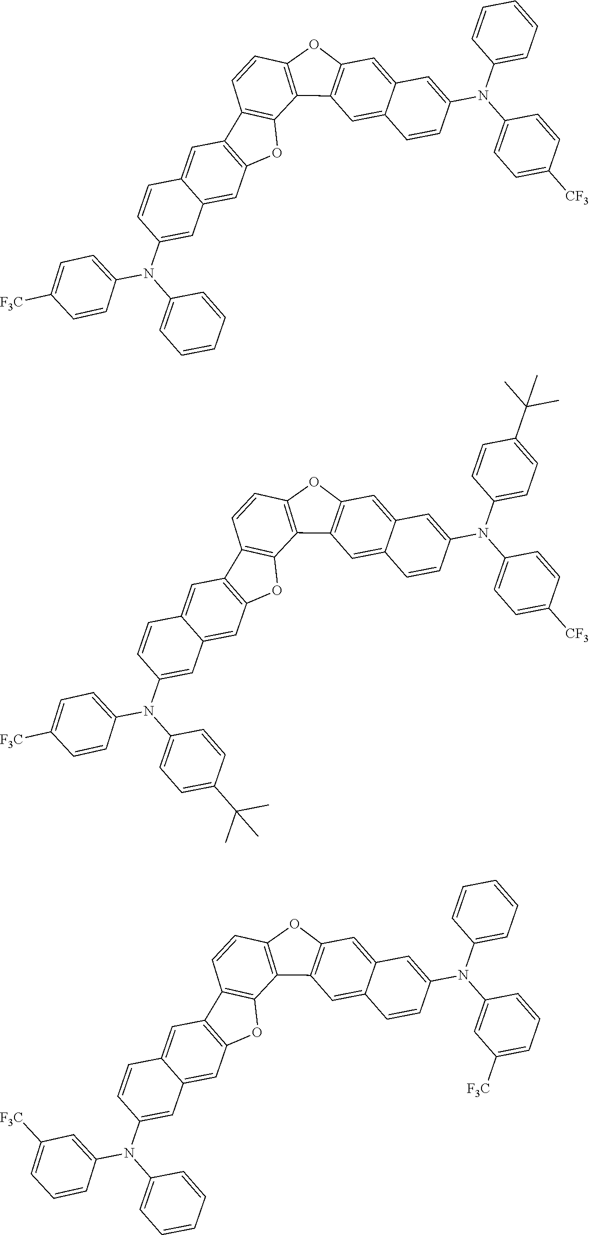 [SMILES: FC(F)(F)c1ccc(N(c2ccccc2)c2ccc3cc4c(cc3c2)oc2c4ccc3oc4cc5cc(N(c6ccccc6)c6ccc(C(F)(F)F)cc6)ccc5cc4c32)cc1 ; CC(C)(C)c1ccc(N(c2ccc(C(F)(F)F)cc2)c2ccc3cc4c(cc3c2)oc2c4ccc3oc4cc5cc(N(c6ccc(C(C)(C)C)cc6)c6ccc(C(F)(F)F)cc6)ccc5cc4c32)cc1 ; FC(F)(F)c1cccc(N(c2ccccc2)c2ccc3cc4c(cc3c2)oc2c4ccc3oc4cc5cc(N(c6ccccc6)c6cccc(C(F)(F)F)c6)ccc5cc4c32)c1]

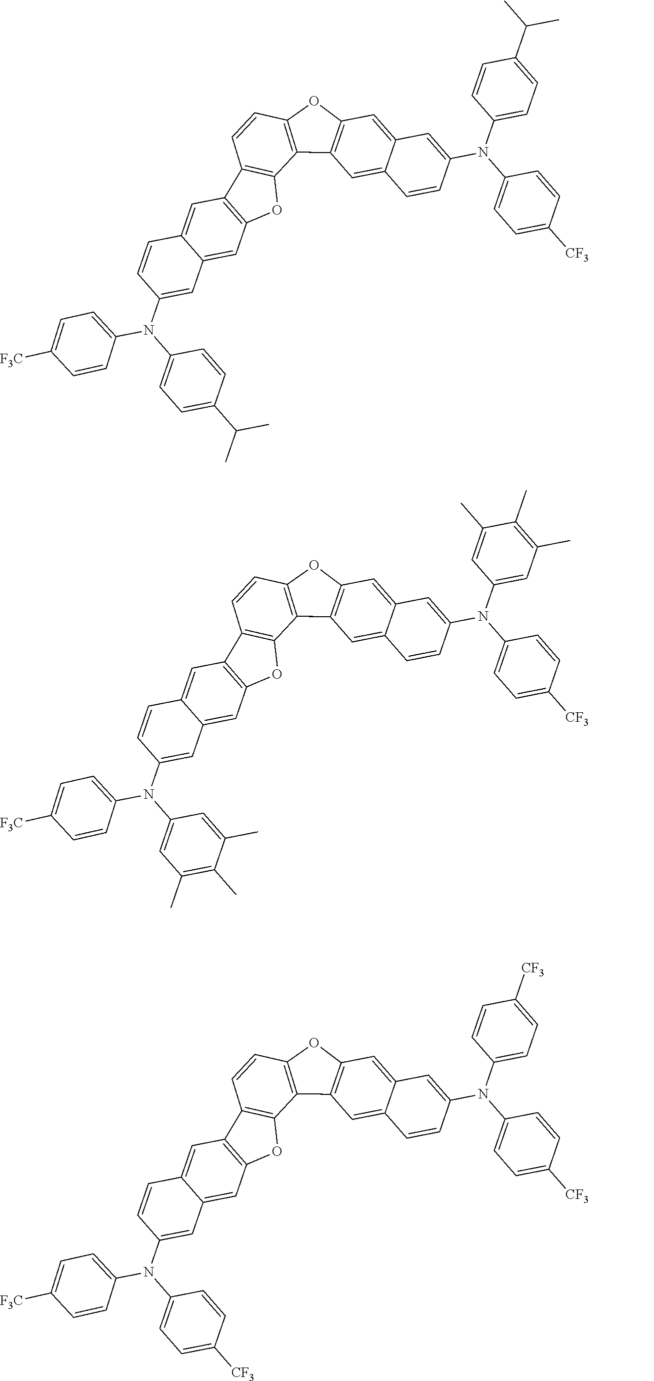 [SMILES: CC(C)c1ccc(N(c2ccc(C(F)(F)F)cc2)c2ccc3cc4c(cc3c2)oc2c4ccc3oc4cc5cc(N(c6ccc(C(C)C)cc6)c6ccc(C(F)(F)F)cc6)ccc5cc4c32)cc1 ; Cc1cc(N(c2ccc(C(F)(F)F)cc2)c2ccc3cc4c(cc3c2)oc2c4ccc3oc4cc5cc(N(c6ccc(C(F)(F)F)cc6)c6cc(C)c(C)c(C)c6)ccc5cc4c32)cc(C)c1C ; FC(F)(F)c1ccc(N(c2ccc(C(F)(F)F)cc2)c2ccc3cc4c(cc3c2)oc2c4ccc3oc4cc5cc(N(c6ccc(C(F)(F)F)cc6)c6ccc(C(F)(F)F)cc6)ccc5cc4c32)cc1]

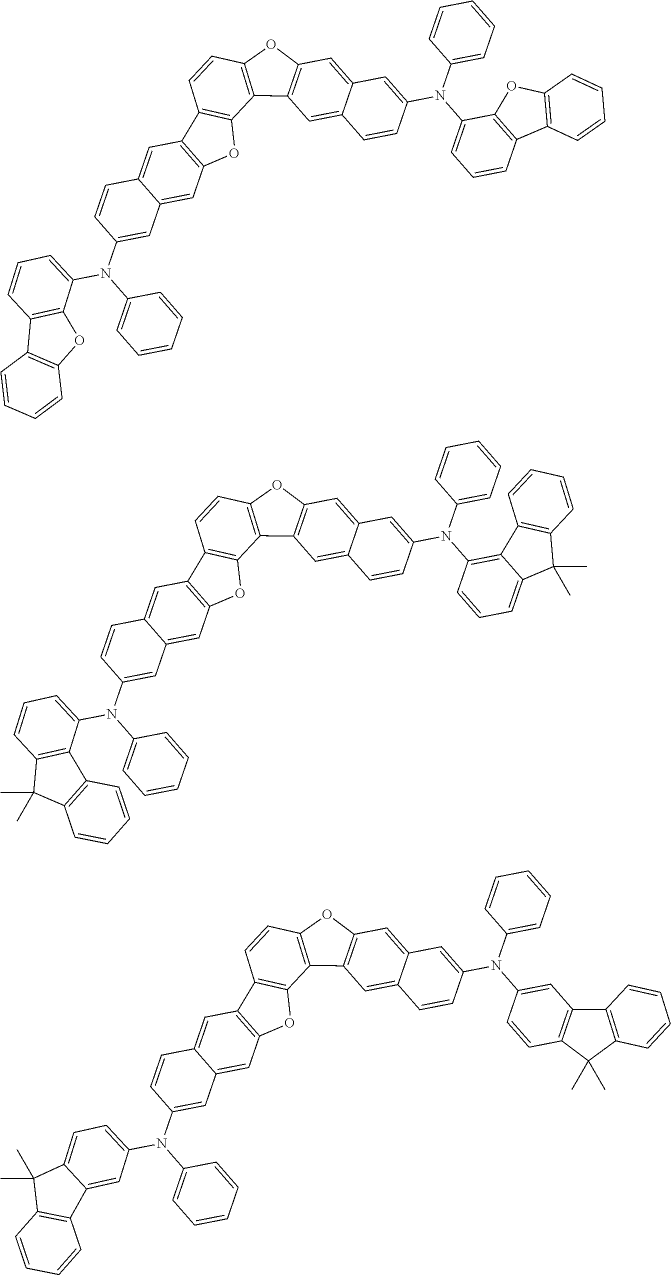

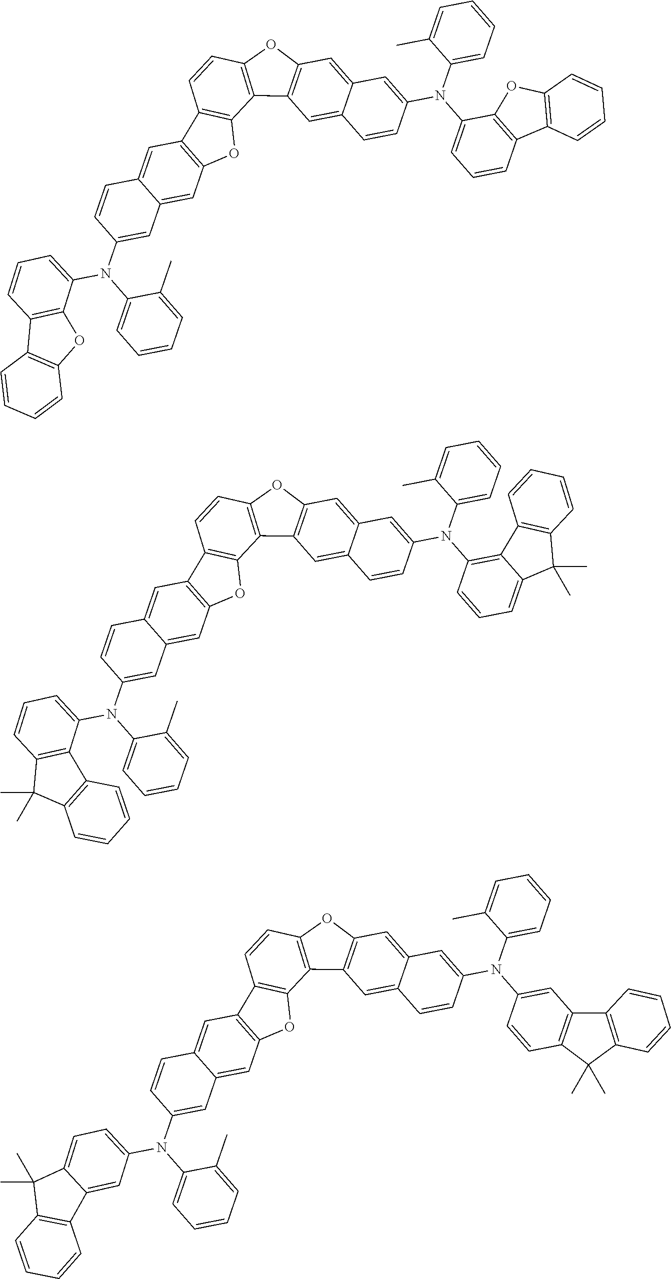

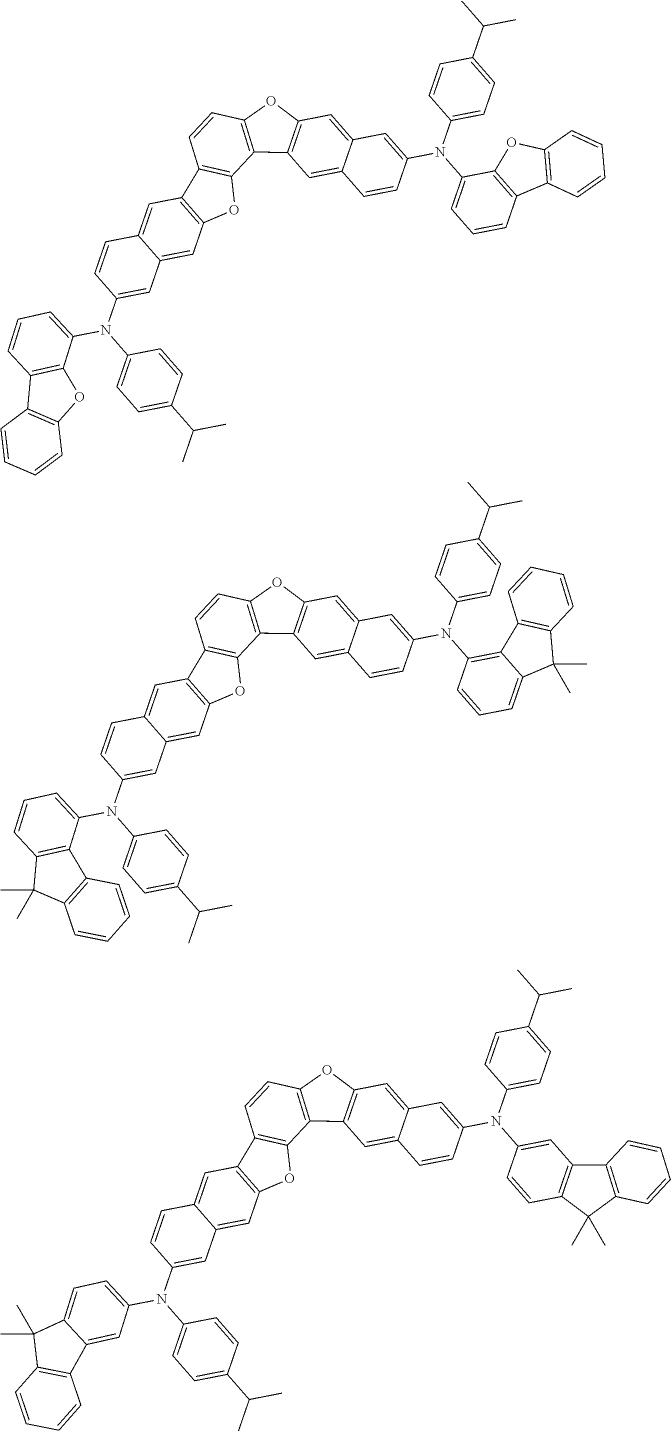

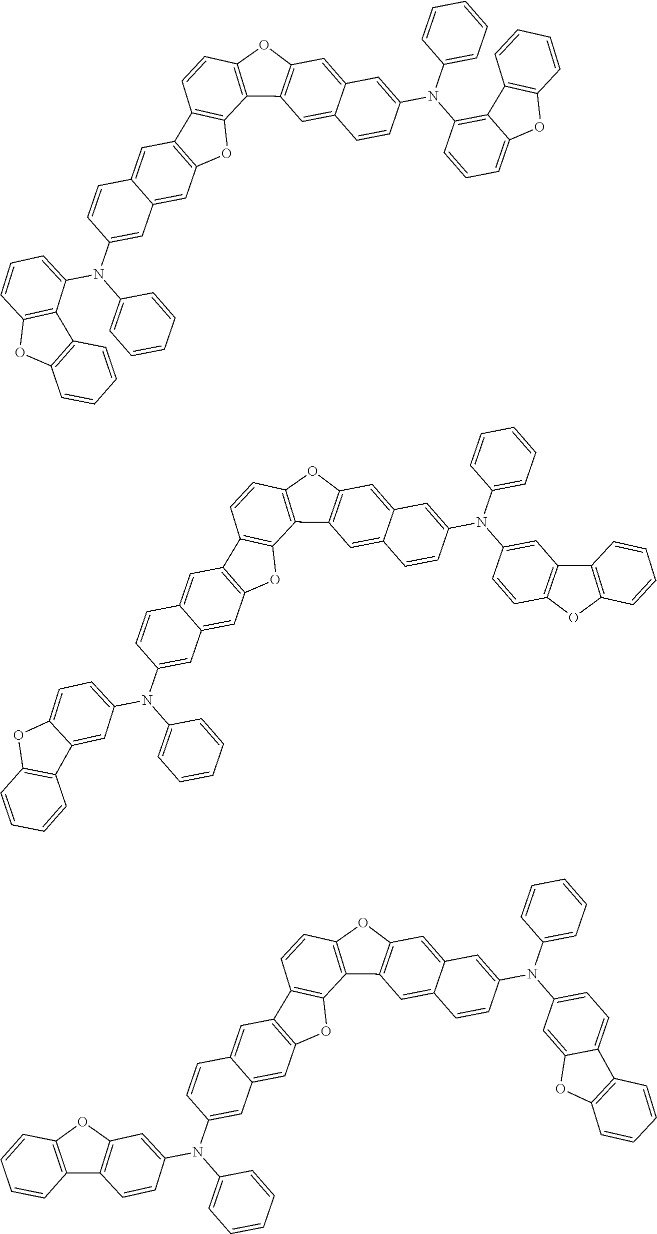 [SMILES: c1ccc(N(c2ccc3cc4c(cc3c2)oc2c4ccc3oc4cc5cc(N(c6ccccc6)c6cccc7oc8ccccc8c67)ccc5cc4c32)c2cccc3oc4ccccc4c23)cc1 ; c1ccc(N(c2ccc3cc4c(cc3c2)oc2c4ccc3oc4cc5cc(N(c6ccccc6)c6ccc7oc8ccccc8c7c6)ccc5cc4c32)c2ccc3oc4ccccc4c3c2)cc1 ; c1ccc(N(c2ccc3cc4c(cc3c2)oc2c4ccc3oc4cc5cc(N(c6ccccc6)c6ccc7c(c6)oc6ccccc67)ccc5cc4c32)c2ccc3c(c2)oc2ccccc23)cc1]

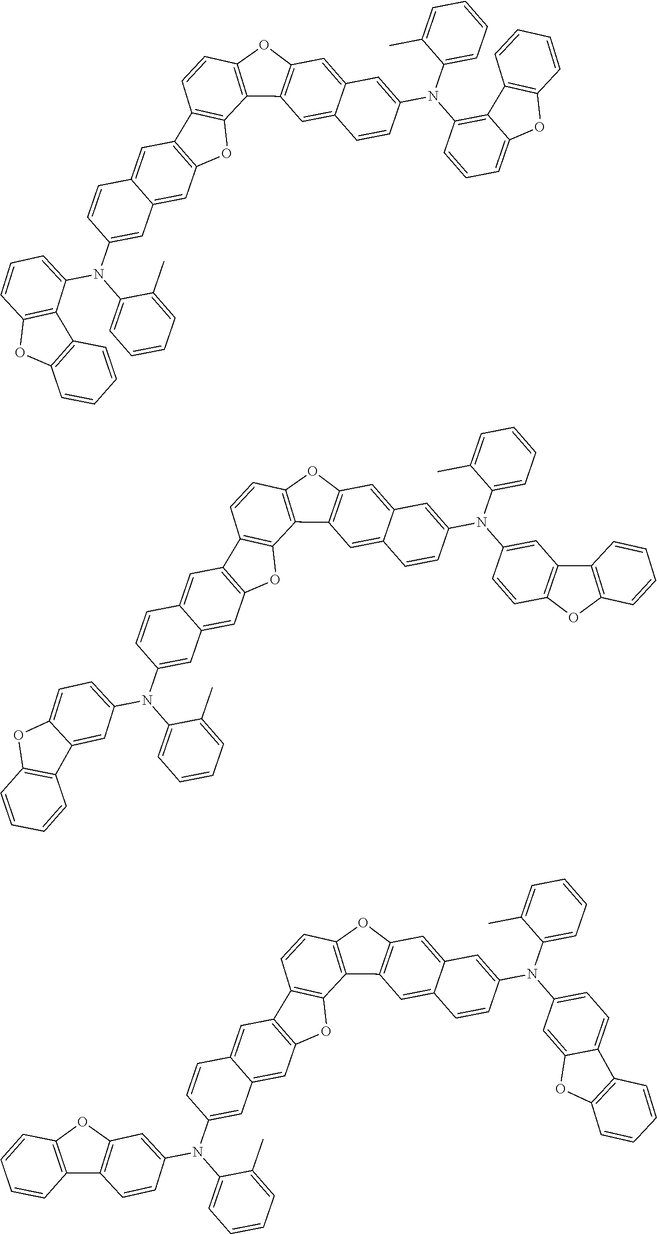 [SMILES: Cc1ccccc1N(c1ccc2cc3c(cc2c1)oc1c3ccc2oc3cc4cc(N(c5ccccc5C)c5cccc6oc7ccccc7c56)ccc4cc3c21)c1cccc2oc3ccccc3c12 ; Cc1ccccc1N(c1ccc2cc3c(cc2c1)oc1c3ccc2oc3cc4cc(N(c5ccc6oc7ccccc7c6c5)c5ccccc5C)ccc4cc3c21)c1ccc2oc3ccccc3c2c1 ; Cc1ccccc1N(c1ccc2cc3c(cc2c1)oc1c3ccc2oc3cc4cc(N(c5ccc6c(c5)oc5ccccc56)c5ccccc5C)ccc4cc3c21)c1ccc2c(c1)oc1ccccc12]

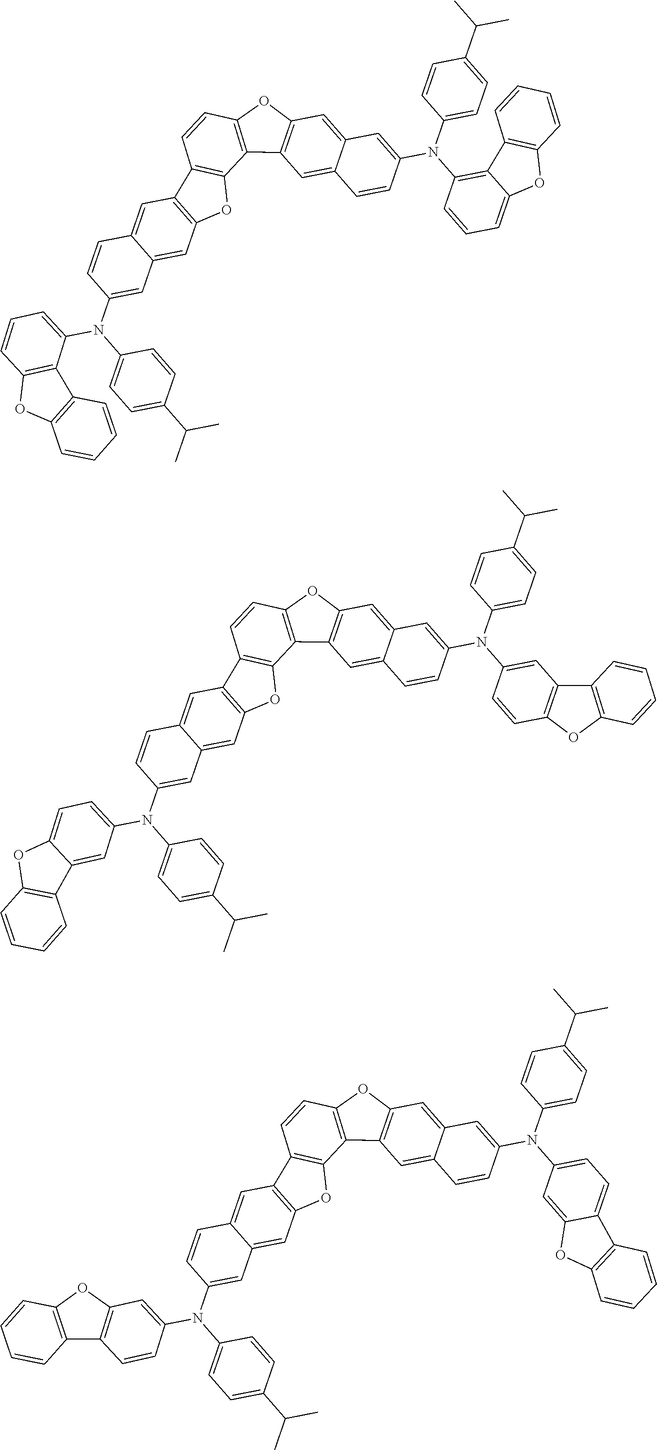 [SMILES: CC(C)c1ccc(N(c2ccc3cc4c(cc3c2)oc2c4ccc3oc4cc5cc(N(c6ccc(C(C)C)cc6)c6cccc7oc8ccccc8c67)ccc5cc4c32)c2cccc3oc4ccccc4c23)cc1 ; CC(C)c1ccc(N(c2ccc3cc4c(cc3c2)oc2c4ccc3oc4cc5cc(N(c6ccc(C(C)C)cc6)c6ccc7oc8ccccc8c7c6)ccc5cc4c32)c2ccc3oc4ccccc4c3c2)cc1 ; CC(C)c1ccc(N(c2ccc3cc4c(cc3c2)oc2c4ccc3oc4cc5cc(N(c6ccc(C(C)C)cc6)c6ccc7c(c6)oc6ccccc67)ccc5cc4c32)c2ccc3c(c2)oc2ccccc23)cc1]

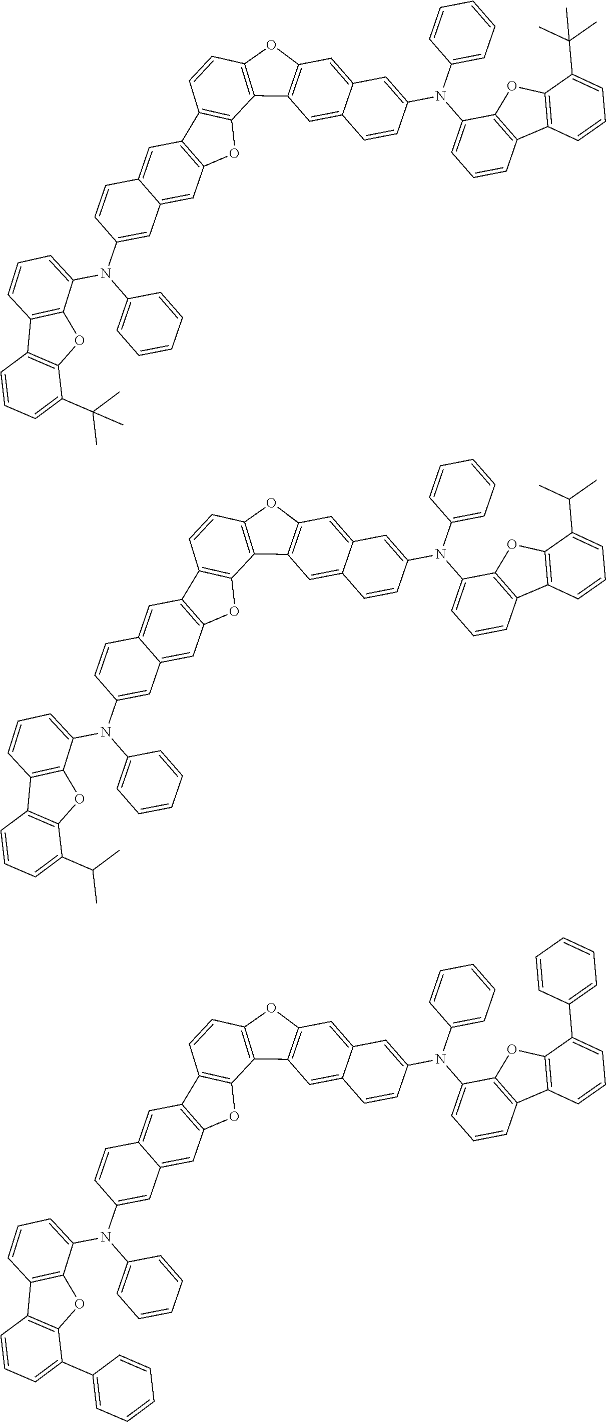 [SMILES: CC(C)(C)c1cccc2c1oc1c(N(c3ccccc3)c3ccc4cc5c(cc4c3)oc3c5ccc4oc5cc6cc(N(c7ccccc7)c7cccc8c7oc7c(C(C)(C)C)cccc78)ccc6cc5c43)cccc12 ; CC(C)c1cccc2c1oc1c(N(c3ccccc3)c3ccc4cc5c(cc4c3)oc3c5ccc4oc5cc6cc(N(c7ccccc7)c7cccc8c7oc7c(C(C)C)cccc78)ccc6cc5c43)cccc12 ; c1ccc(-c2cccc3c2oc2c(N(c4ccccc4)c4ccc5cc6c(cc5c4)oc4c6ccc5oc6cc7cc(N(c8ccccc8)c8cccc9c8oc8c(-c%10ccccc%10)cccc89)ccc7cc6c54)cccc23)cc1]

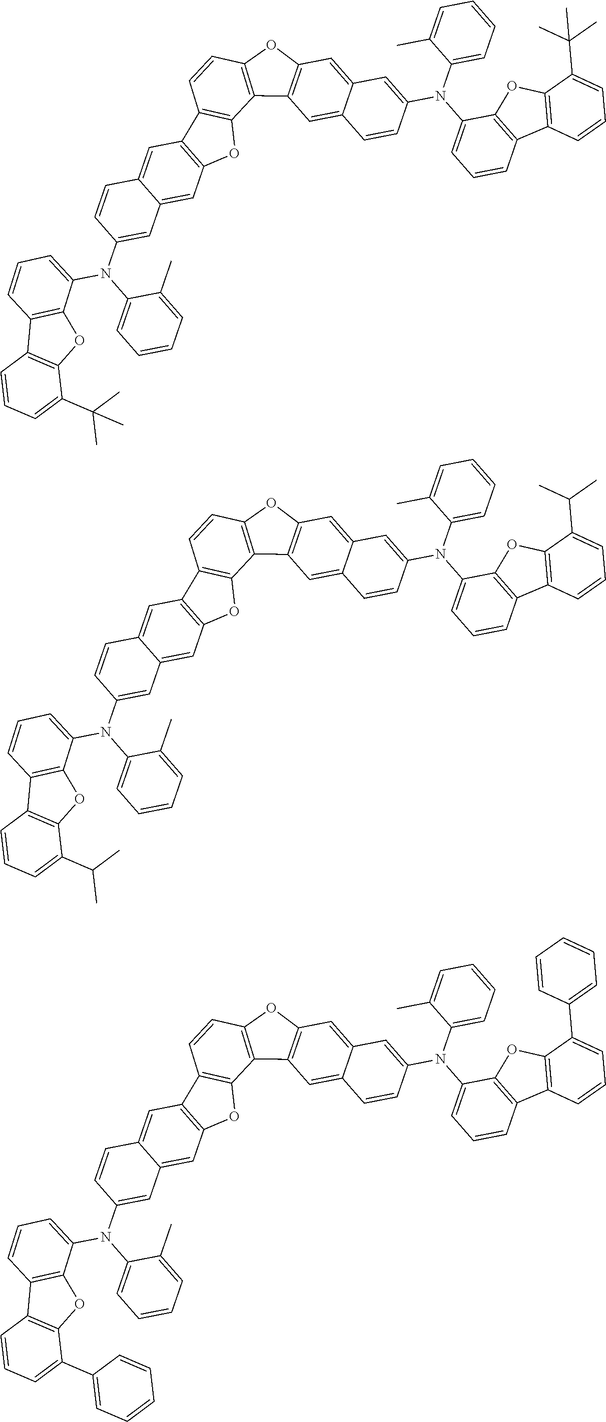 [SMILES: Cc1ccccc1N(c1ccc2cc3c(cc2c1)oc1c3ccc2oc3cc4cc(N(c5ccccc5C)c5cccc6c5oc5c(C(C)(C)C)cccc56)ccc4cc3c21)c1cccc2c1oc1c(C(C)(C)C)cccc12 ; Cc1ccccc1N(c1ccc2cc3c(cc2c1)oc1c3ccc2oc3cc4cc(N(c5ccccc5C)c5cccc6c5oc5c(C(C)C)cccc56)ccc4cc3c21)c1cccc2c1oc1c(C(C)C)cccc12 ; Cc1ccccc1N(c1ccc2cc3c(cc2c1)oc1c3ccc2oc3cc4cc(N(c5ccccc5C)c5cccc6c5oc5c(-c7ccccc7)cccc56)ccc4cc3c21)c1cccc2c1oc1c(-c3ccccc3)cccc12]

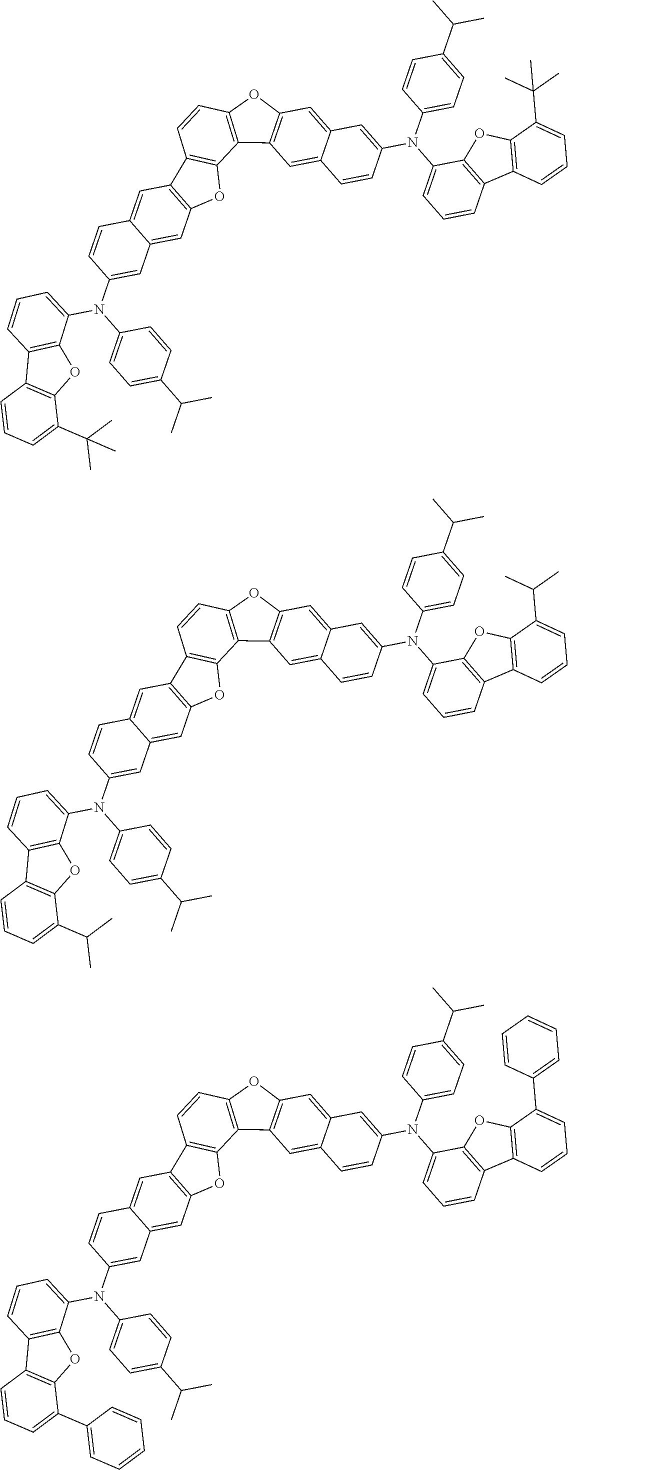 [SMILES: CC(C)c1ccc(N(c2ccc3cc4c(cc3c2)oc2c4ccc3oc4cc5cc(N(c6ccc(C(C)C)cc6)c6cccc7c6oc6c(C(C)(C)C)cccc67)ccc5cc4c32)c2cccc3c2oc2c(C(C)(C)C)cccc23)cc1 ; CC(C)c1ccc(N(c2ccc3cc4c(cc3c2)oc2c4ccc3oc4cc5cc(N(c6ccc(C(C)C)cc6)c6cccc7c6oc6c(C(C)C)cccc67)ccc5cc4c32)c2cccc3c2oc2c(C(C)C)cccc23)cc1 ; CC(C)c1ccc(N(c2ccc3cc4c(cc3c2)oc2c4ccc3oc4cc5cc(N(c6ccc(C(C)C)cc6)c6cccc7c6oc6c(-c8ccccc8)cccc67)ccc5cc4c32)c2cccc3c2oc2c(-c4ccccc4)cccc23)cc1]

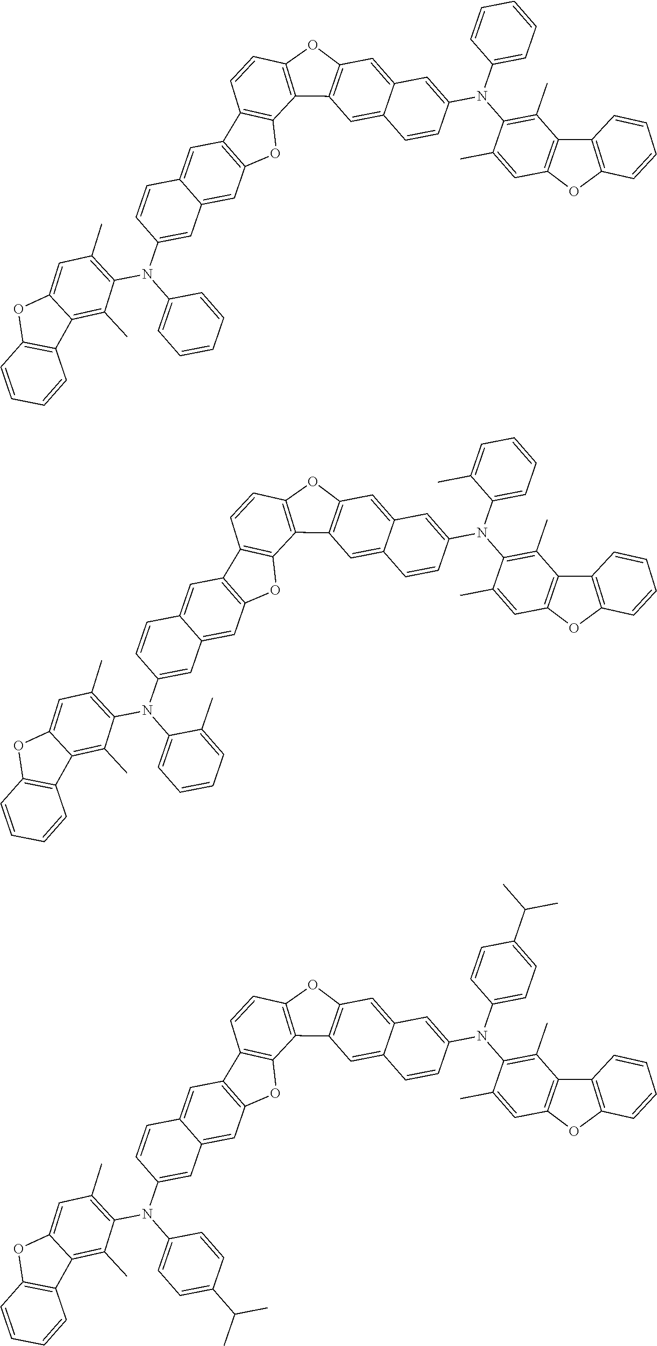 [SMILES: Cc1cc2oc3ccccc3c2c(C)c1N(c1ccccc1)c1ccc2cc3c(cc2c1)oc1c3ccc2oc3cc4cc(N(c5ccccc5)c5c(C)cc6oc7ccccc7c6c5C)ccc4cc3c21 ; Cc1ccccc1N(c1ccc2cc3c(cc2c1)oc1c3ccc2oc3cc4cc(N(c5ccccc5C)c5c(C)cc6oc7ccccc7c6c5C)ccc4cc3c21)c1c(C)cc2oc3ccccc3c2c1C ; Cc1cc2oc3ccccc3c2c(C)c1N(c1ccc(C(C)C)cc1)c1ccc2cc3c(cc2c1)oc1c3ccc2oc3cc4cc(N(c5ccc(C(C)C)cc5)c5c(C)cc6oc7ccccc7c6c5C)ccc4cc3c21]

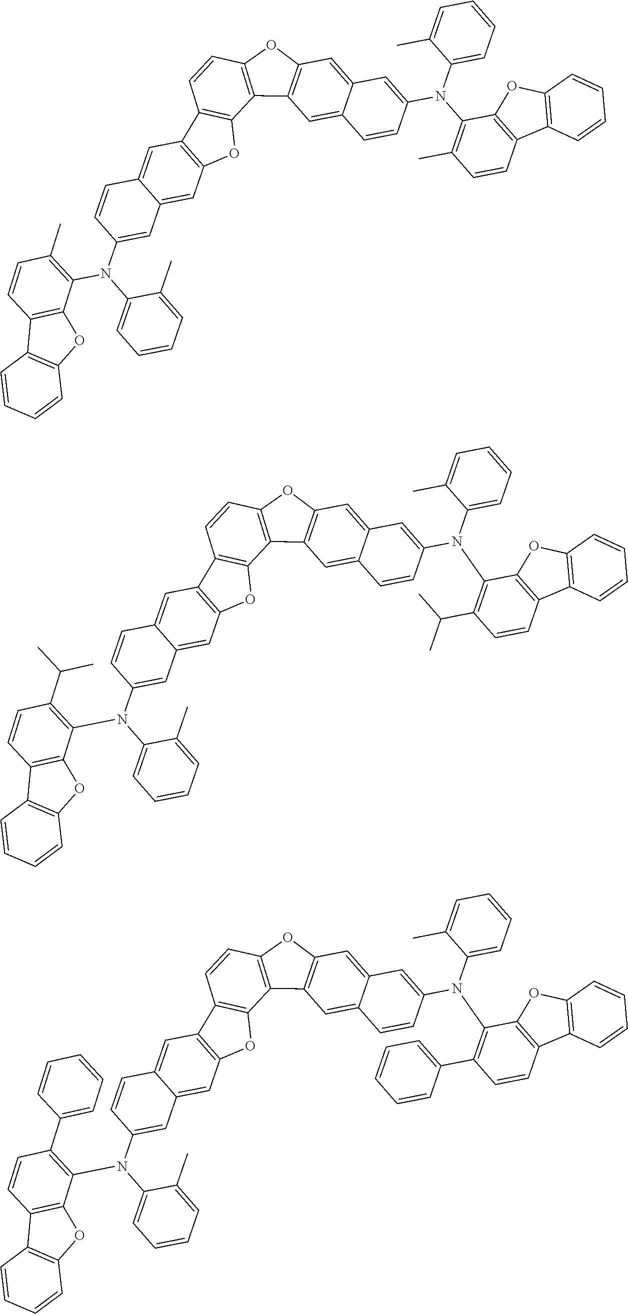 [SMILES: Cc1ccccc1N(c1ccc2cc3c(cc2c1)oc1c3ccc2oc3cc4cc(N(c5ccccc5C)c5c(C)ccc6c5oc5ccccc56)ccc4cc3c21)c1c(C)ccc2c1oc1ccccc12 ; Cc1ccccc1N(c1ccc2cc3c(cc2c1)oc1c3ccc2oc3cc4cc(N(c5ccccc5C)c5c(C(C)C)ccc6c5oc5ccccc56)ccc4cc3c21)c1c(C(C)C)ccc2c1oc1ccccc12 ; Cc1ccccc1N(c1ccc2cc3c(cc2c1)oc1c3ccc2oc3cc4cc(N(c5ccccc5C)c5c(-c6ccccc6)ccc6c5oc5ccccc56)ccc4cc3c21)c1c(-c2ccccc2)ccc2c1oc1ccccc12]

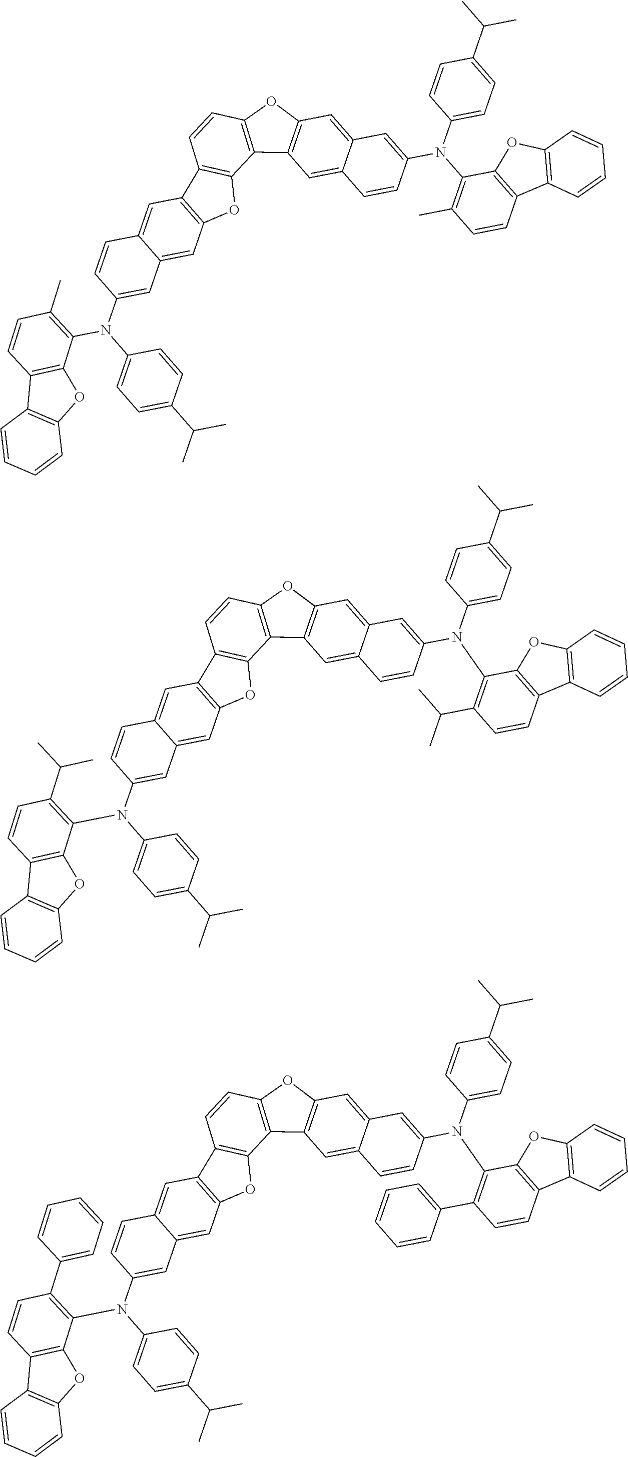 [SMILES: Cc1ccc2c(oc3ccccc32)c1N(c1ccc(C(C)C)cc1)c1ccc2cc3c(cc2c1)oc1c3ccc2oc3cc4cc(N(c5ccc(C(C)C)cc5)c5c(C)ccc6c5oc5ccccc56)ccc4cc3c21 ; CC(C)c1ccc(N(c2ccc3cc4c(cc3c2)oc2c4ccc3oc4cc5cc(N(c6ccc(C(C)C)cc6)c6c(C(C)C)ccc7c6oc6ccccc67)ccc5cc4c32)c2c(C(C)C)ccc3c2oc2ccccc23)cc1 ; CC(C)c1ccc(N(c2ccc3cc4c(cc3c2)oc2c4ccc3oc4cc5cc(N(c6ccc(C(C)C)cc6)c6c(-c7ccccc7)ccc7c6oc6ccccc67)ccc5cc4c32)c2c(-c3ccccc3)ccc3c2oc2ccccc23)cc1]

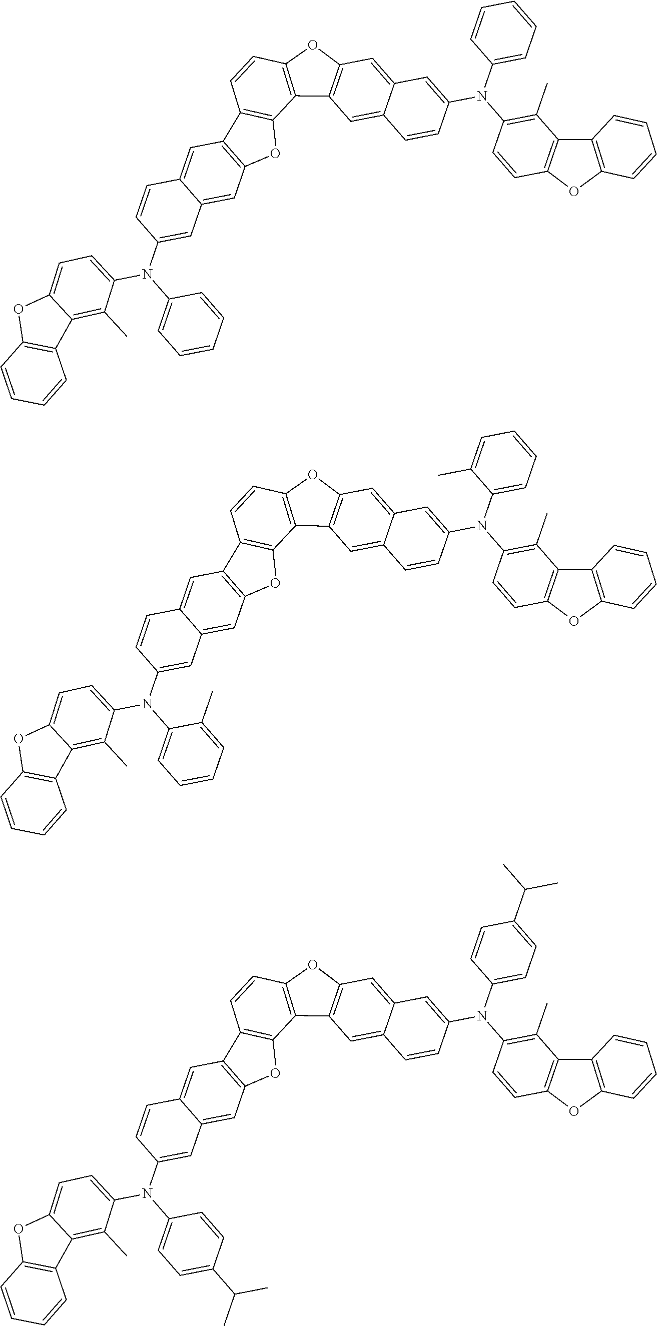 [SMILES: Cc1c(N(c2ccccc2)c2ccc3cc4c(cc3c2)oc2c4ccc3oc4cc5cc(N(c6ccccc6)c6ccc7oc8ccccc8c7c6C)ccc5cc4c32)ccc2oc3ccccc3c12 ; Cc1ccccc1N(c1ccc2cc3c(cc2c1)oc1c3ccc2oc3cc4cc(N(c5ccccc5C)c5ccc6oc7ccccc7c6c5C)ccc4cc3c21)c1ccc2oc3ccccc3c2c1C ; Cc1c(N(c2ccc(C(C)C)cc2)c2ccc3cc4c(cc3c2)oc2c4ccc3oc4cc5cc(N(c6ccc(C(C)C)cc6)c6ccc7oc8ccccc8c7c6C)ccc5cc4c32)ccc2oc3ccccc3c12]

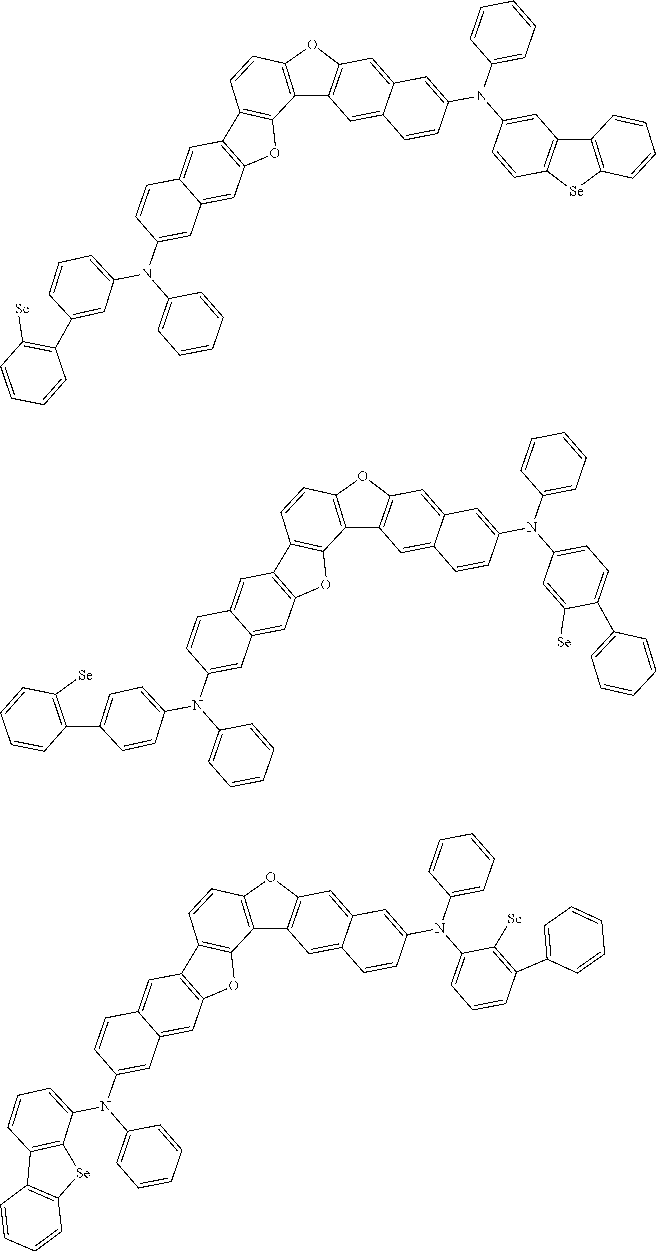

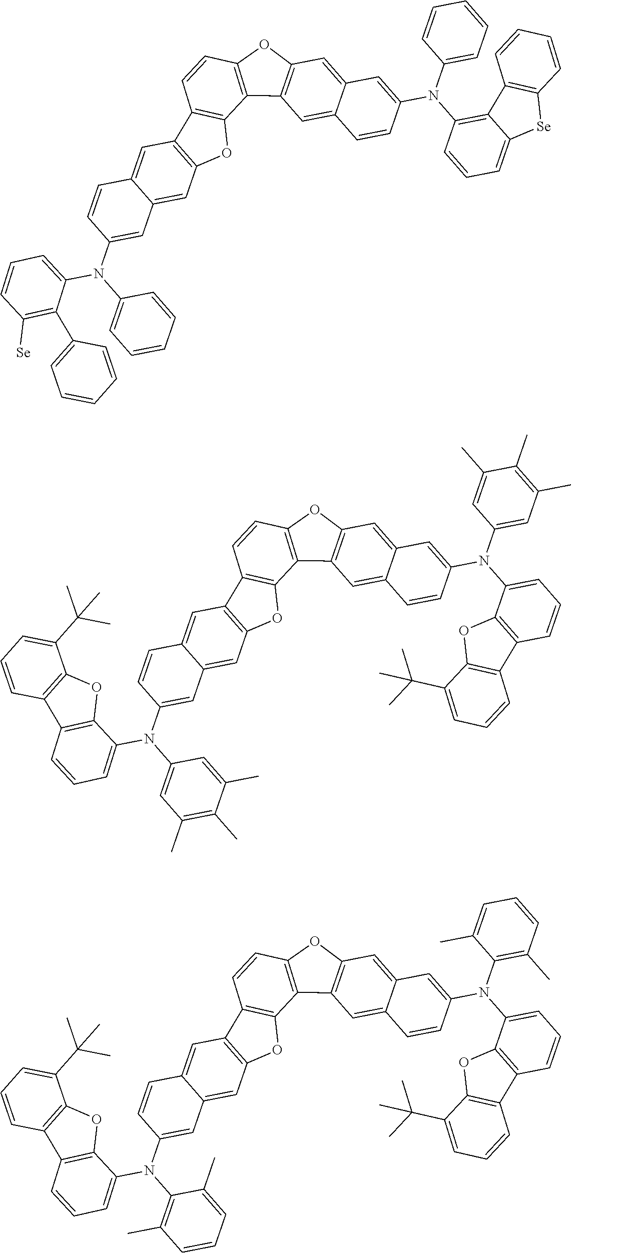

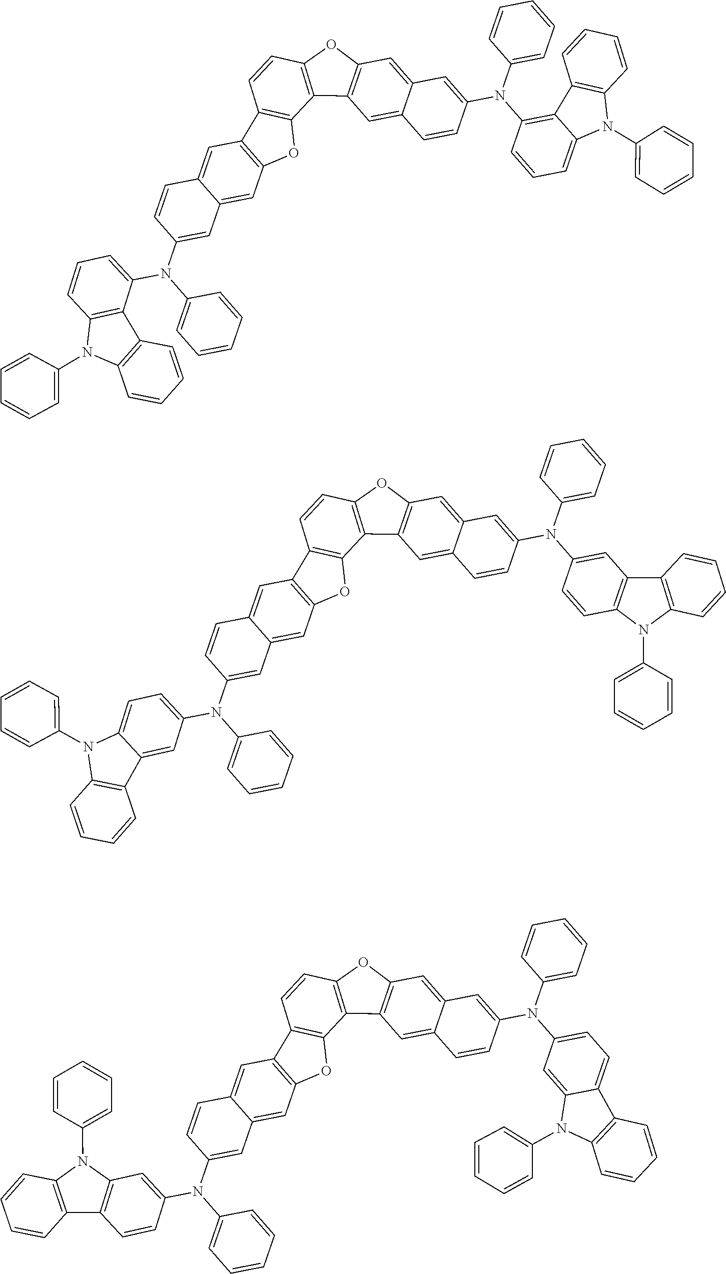

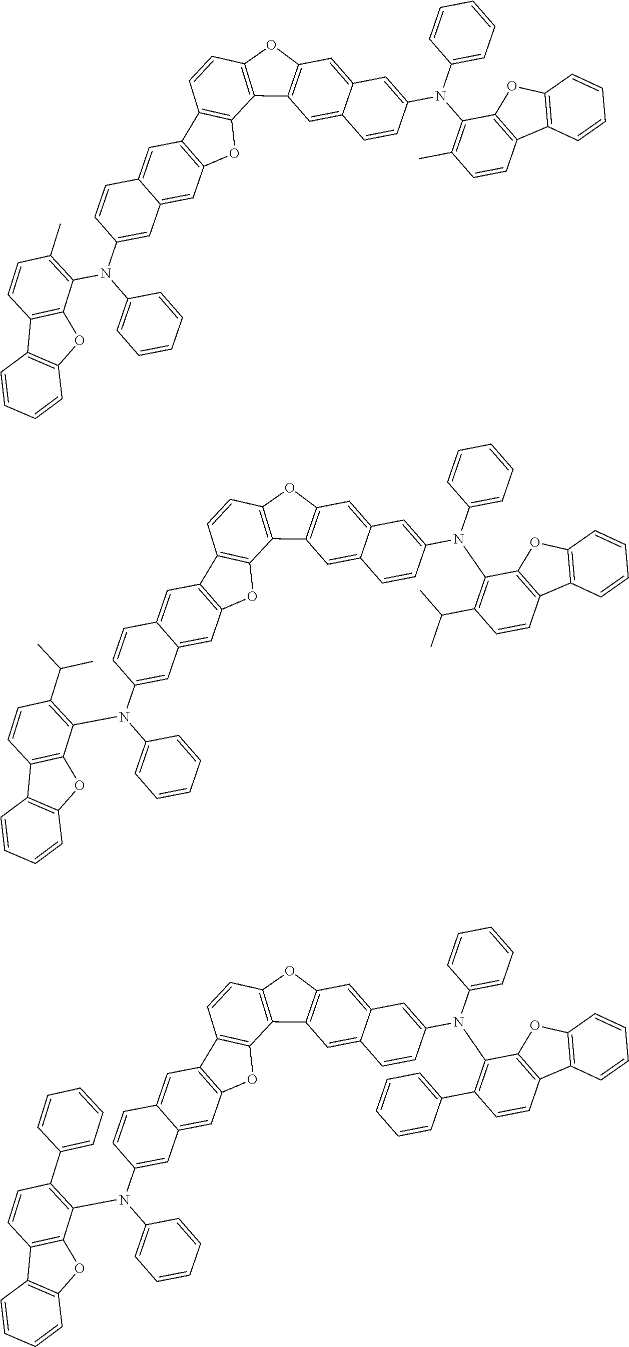 [SMILES: Cc1ccc2c(oc3ccccc32)c1N(c1ccccc1)c1ccc2cc3c(cc2c1)oc1c3ccc2oc3cc4cc(N(c5ccccc5)c5c(C)ccc6c5oc5ccccc56)ccc4cc3c21 ; CC(C)c1ccc2c(oc3ccccc32)c1N(c1ccccc1)c1ccc2cc3c(cc2c1)oc1c3ccc2oc3cc4cc(N(c5ccccc5)c5c(C(C)C)ccc6c5oc5ccccc56)ccc4cc3c21 ; c1ccc(-c2ccc3c(oc4ccccc43)c2N(c2ccccc2)c2ccc3cc4c(cc3c2)oc2c4ccc3oc4cc5cc(N(c6ccccc6)c6c(-c7ccccc7)ccc7c6oc6ccccc67)ccc5cc4c32)cc1]

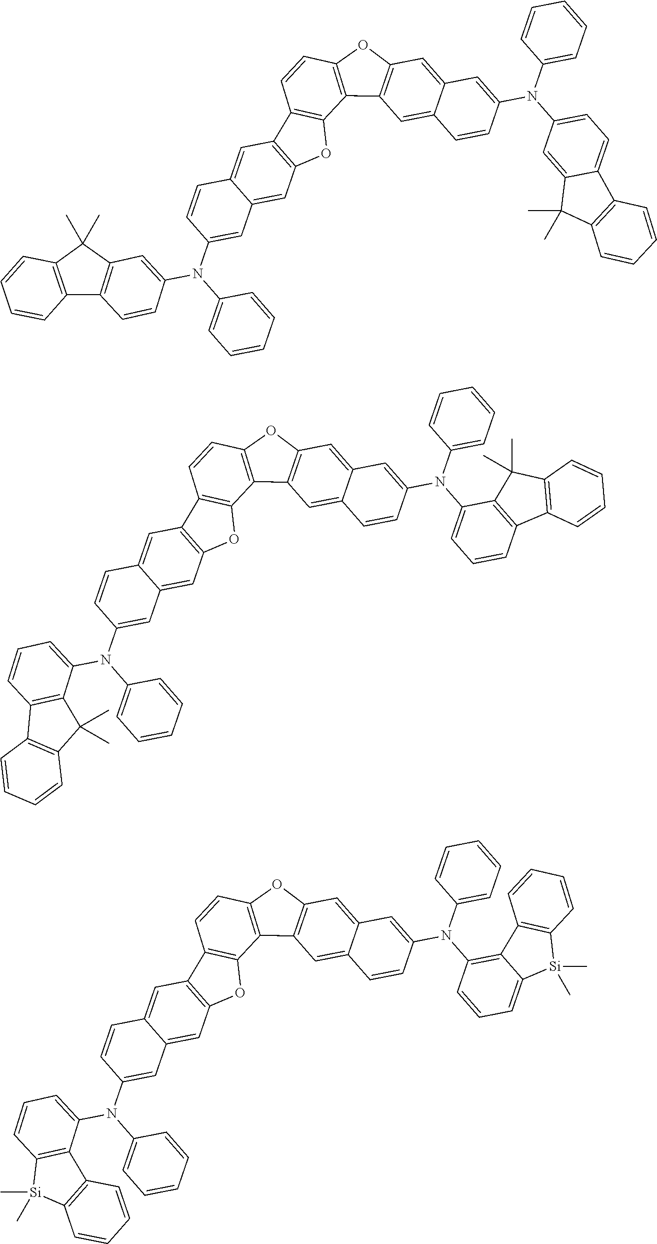

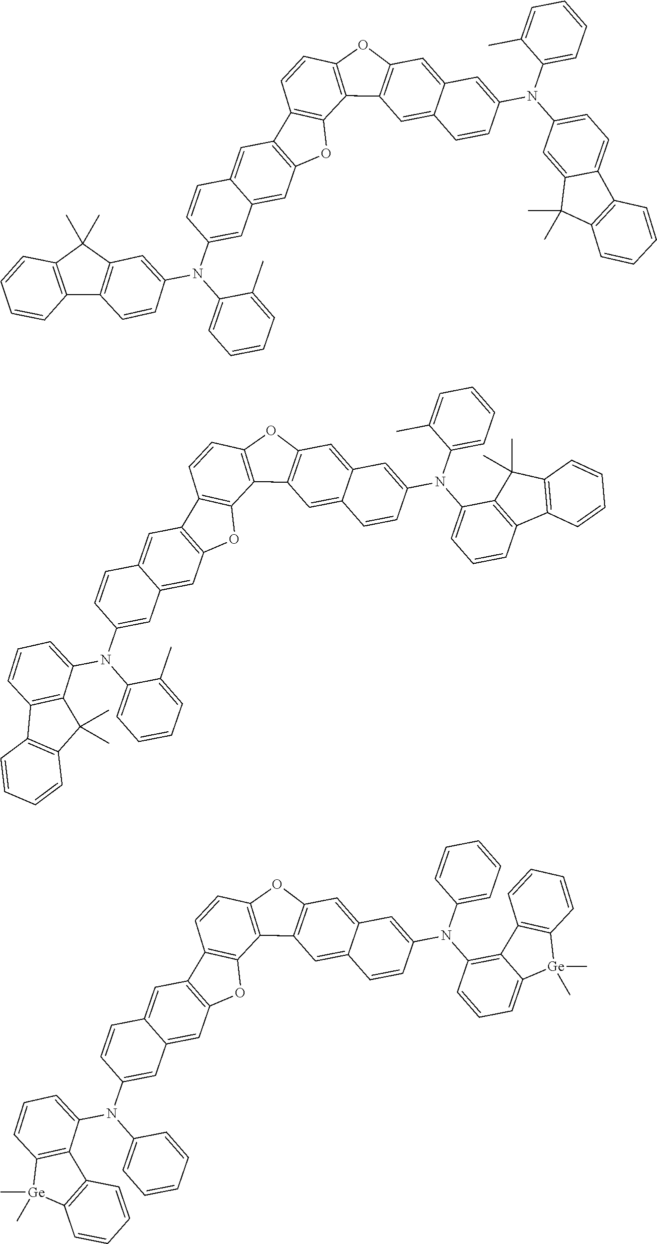

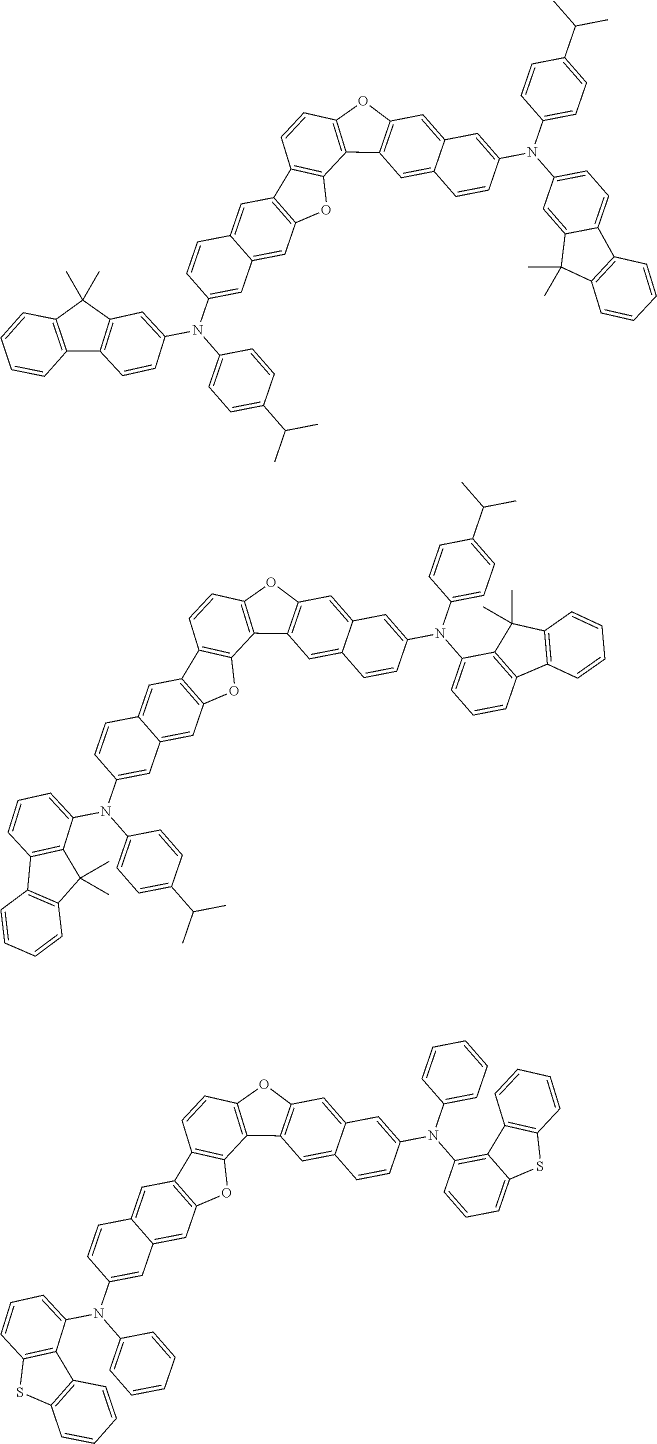 [SMILES: CC(C)c1ccc(N(c2ccc3c(c2)C(C)(C)c2ccccc2-3)c2ccc3cc4c(cc3c2)oc2c4ccc3oc4cc5cc(N(c6ccc(C(C)C)cc6)c6ccc7c(c6)C(C)(C)c6ccccc6-7)ccc5cc4c32)cc1 ; CC(C)c1ccc(N(c2ccc3cc4c(cc3c2)oc2c4ccc3oc4cc5cc(N(c6ccc(C(C)C)cc6)c6cccc7c6C(C)(C)c6ccccc6-7)ccc5cc4c32)c2cccc3c2C(C)(C)c2ccccc2-3)cc1 ; c1ccc(N(c2ccc3cc4c(cc3c2)oc2c4ccc3oc4cc5cc(N(c6ccccc6)c6cccc7sc8ccccc8c67)ccc5cc4c32)c2cccc3sc4ccccc4c23)cc1]

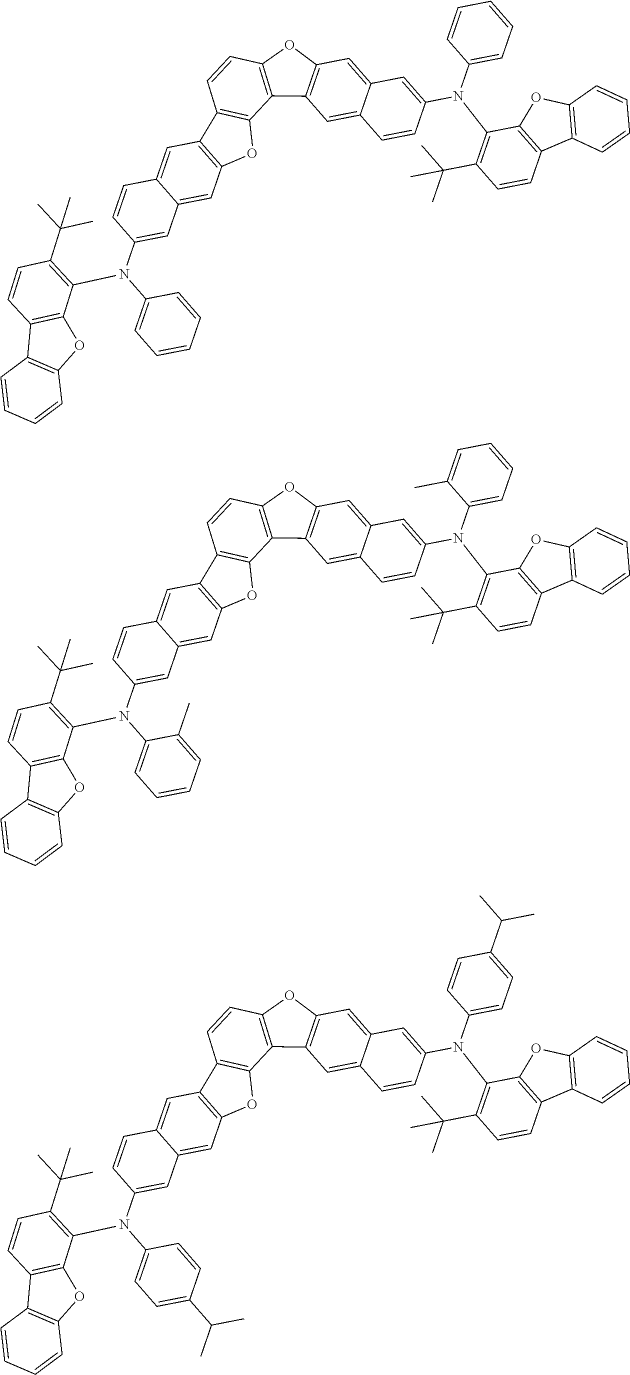 [SMILES: CC(C)(C)c1ccc2c(oc3ccccc32)c1N(c1ccccc1)c1ccc2cc3c(cc2c1)oc1c3ccc2oc3cc4cc(N(c5ccccc5)c5c(C(C)(C)C)ccc6c5oc5ccccc56)ccc4cc3c21 ; Cc1ccccc1N(c1ccc2cc3c(cc2c1)oc1c3ccc2oc3cc4cc(N(c5ccccc5C)c5c(C(C)(C)C)ccc6c5oc5ccccc56)ccc4cc3c21)c1c(C(C)(C)C)ccc2c1oc1ccccc12 ; CC(C)c1ccc(N(c2ccc3cc4c(cc3c2)oc2c4ccc3oc4cc5cc(N(c6ccc(C(C)C)cc6)c6c(C(C)(C)C)ccc7c6oc6ccccc67)ccc5cc4c32)c2c(C(C)(C)C)ccc3c2oc2ccccc23)cc1]

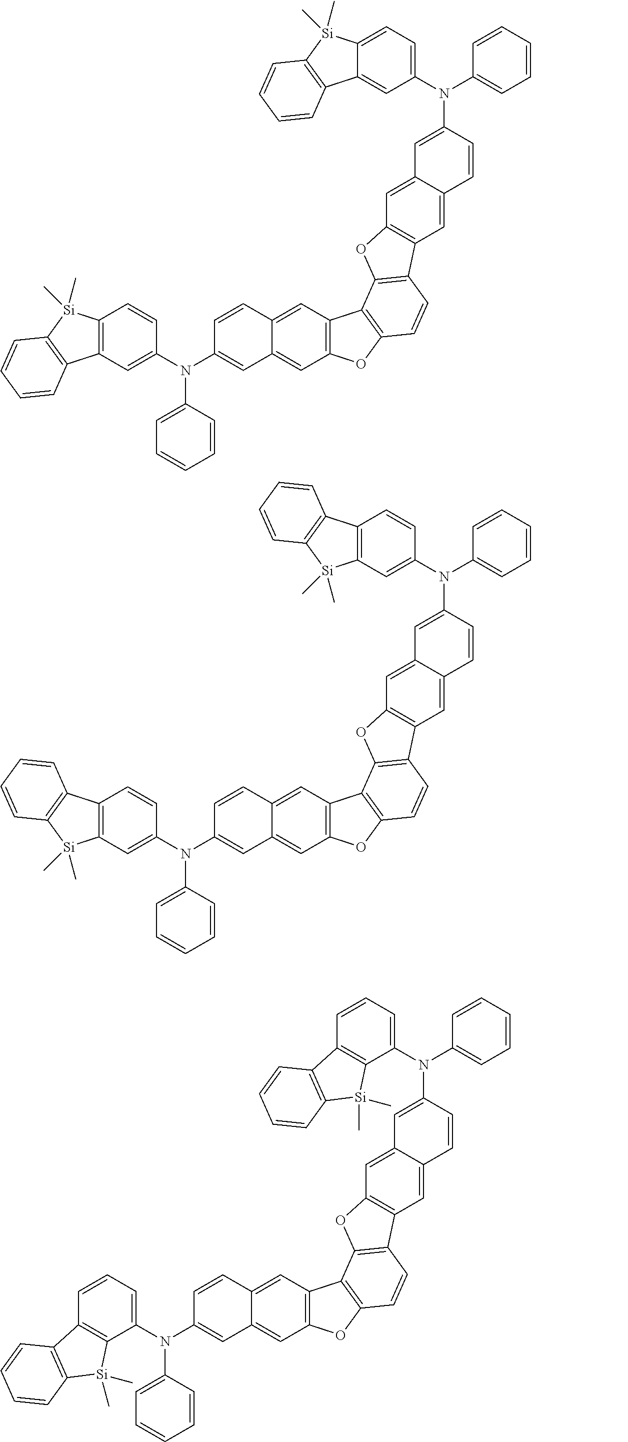 [SMILES: C[Si]1(C)c2ccccc2-c2cc(N(c3ccccc3)c3ccc4cc5c(cc4c3)oc3c5ccc4oc5cc6cc(N(c7ccccc7)c7ccc8c(c7)-c7ccccc7[Si]8(C)C)ccc6cc5c43)ccc21 ; C[Si]1(C)c2ccccc2-c2ccc(N(c3ccccc3)c3ccc4cc5c(cc4c3)oc3c5ccc4oc5cc6cc(N(c7ccccc7)c7ccc8c(c7)[Si](C)(C)c7ccccc7-8)ccc6cc5c43)cc21 ; C[Si]1(C)c2ccccc2-c2cccc(N(c3ccccc3)c3ccc4cc5c(cc4c3)oc3c5ccc4oc5cc6cc(N(c7ccccc7)c7cccc8c7[Si](C)(C)c7ccccc7-8)ccc6cc5c43)c21]

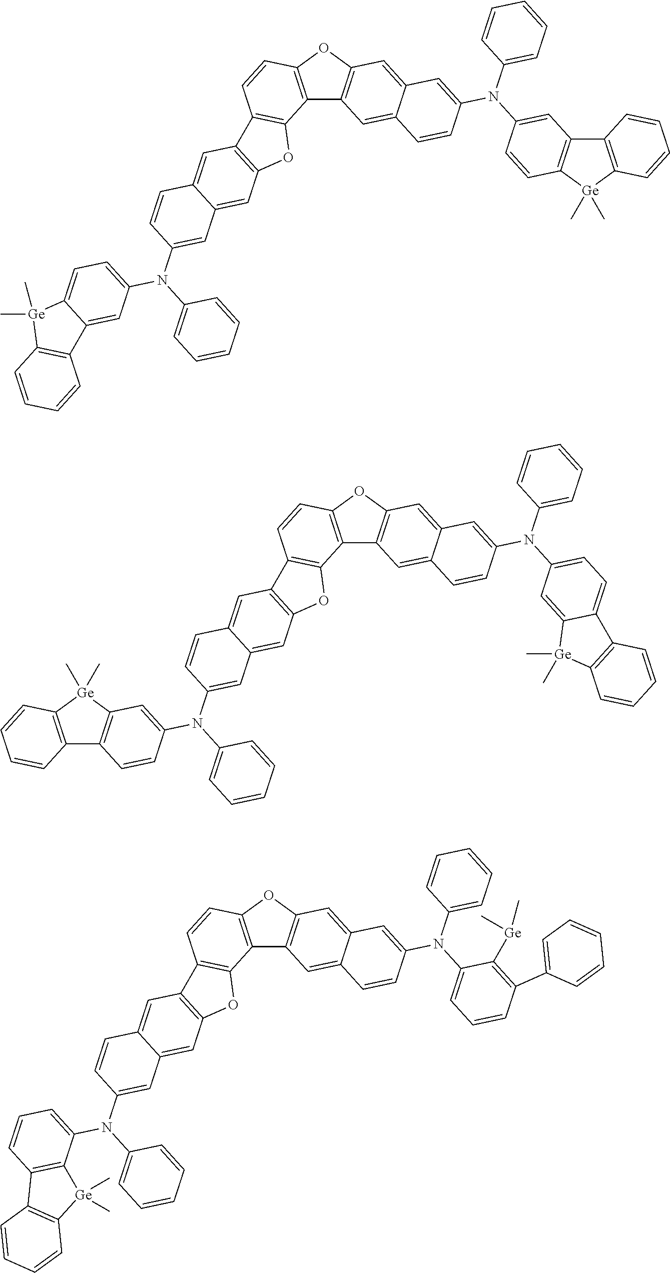

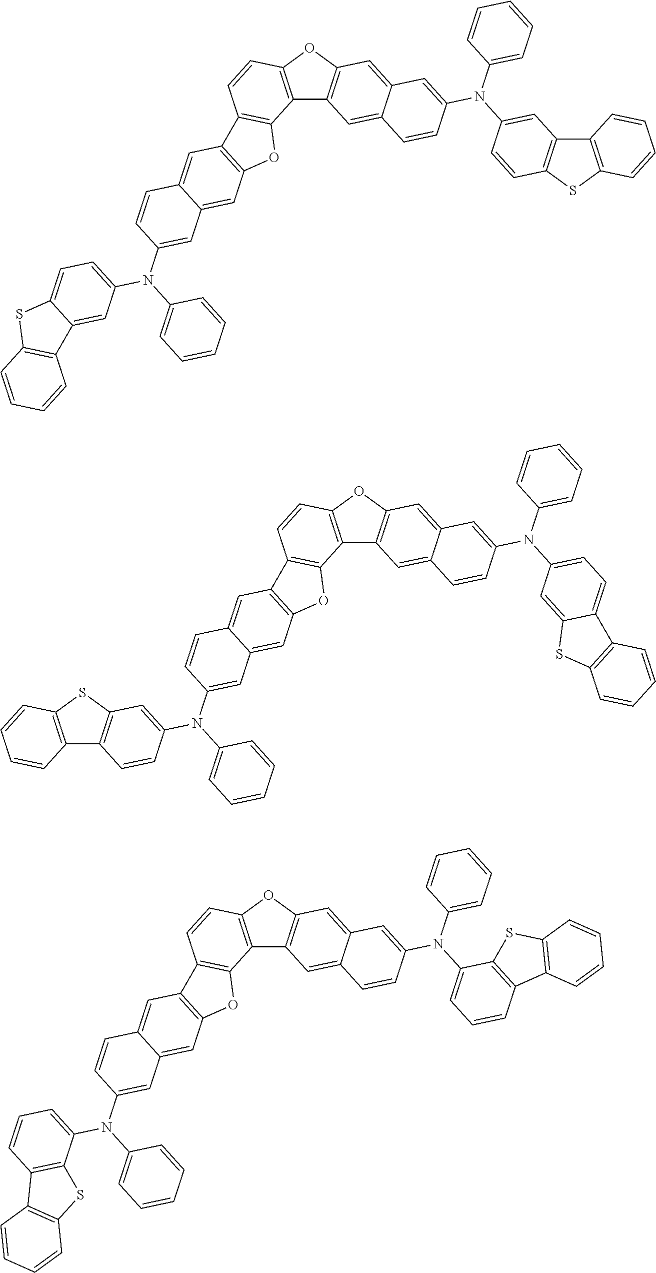 [SMILES: c1ccc(N(c2ccc3cc4c(cc3c2)oc2c4ccc3oc4cc5cc(N(c6ccccc6)c6ccc7sc8ccccc8c7c6)ccc5cc4c32)c2ccc3sc4ccccc4c3c2)cc1 ; c1ccc(N(c2ccc3cc4c(cc3c2)oc2c4ccc3oc4cc5cc(N(c6ccccc6)c6ccc7c(c6)sc6ccccc67)ccc5cc4c32)c2ccc3c(c2)sc2ccccc23)cc1 ; c1ccc(N(c2ccc3cc4c(cc3c2)oc2c4ccc3oc4cc5cc(N(c6ccccc6)c6cccc7c6sc6ccccc67)ccc5cc4c32)c2cccc3c2sc2ccccc23)cc1]

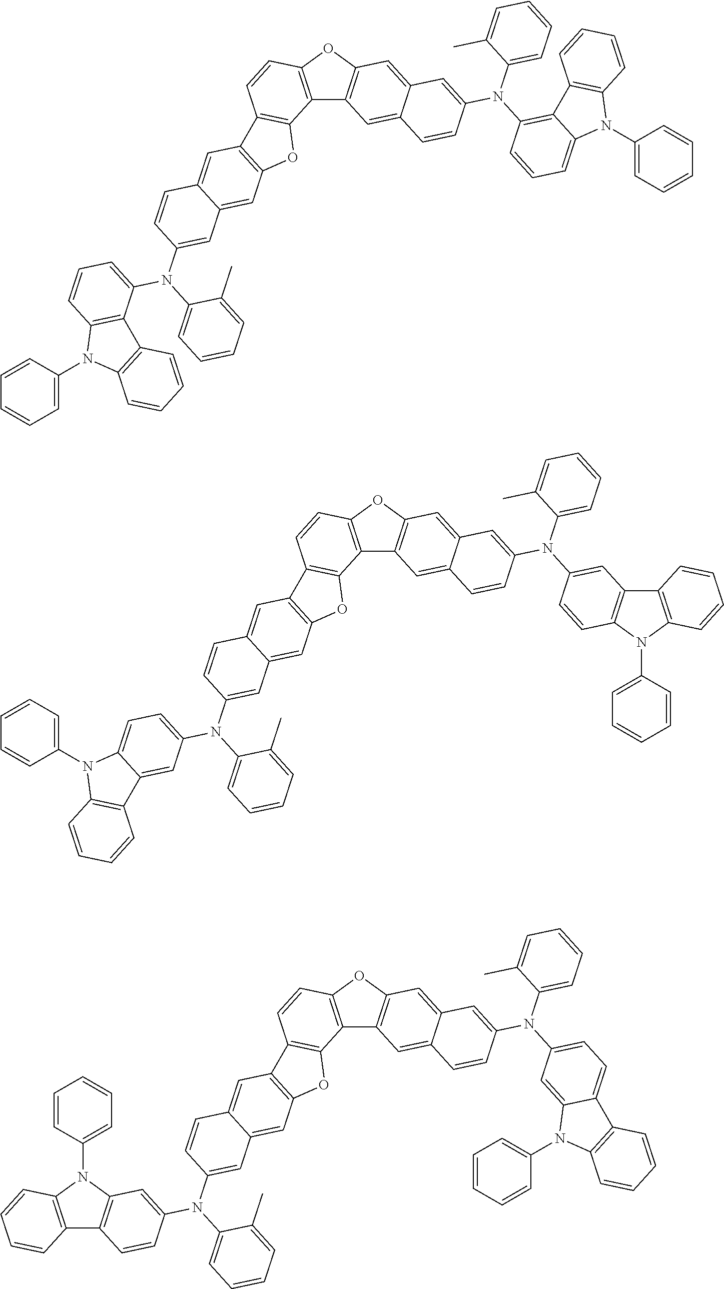 [SMILES: Cc1ccccc1N(c1ccc2cc3c(cc2c1)oc1c3ccc2oc3cc4cc(N(c5ccccc5C)c5cccc6c5c5ccccc5n6-c5ccccc5)ccc4cc3c21)c1cccc2c1c1ccccc1n2-c1ccccc1 ; Cc1ccccc1N(c1ccc2cc3c(cc2c1)oc1c3ccc2oc3cc4cc(N(c5ccc6c(c5)c5ccccc5n6-c5ccccc5)c5ccccc5C)ccc4cc3c21)c1ccc2c(c1)c1ccccc1n2-c1ccccc1 ; Cc1ccccc1N(c1ccc2cc3c(cc2c1)oc1c3ccc2oc3cc4cc(N(c5ccc6c7ccccc7n(-c7ccccc7)c6c5)c5ccccc5C)ccc4cc3c21)c1ccc2c3ccccc3n(-c3ccccc3)c2c1]

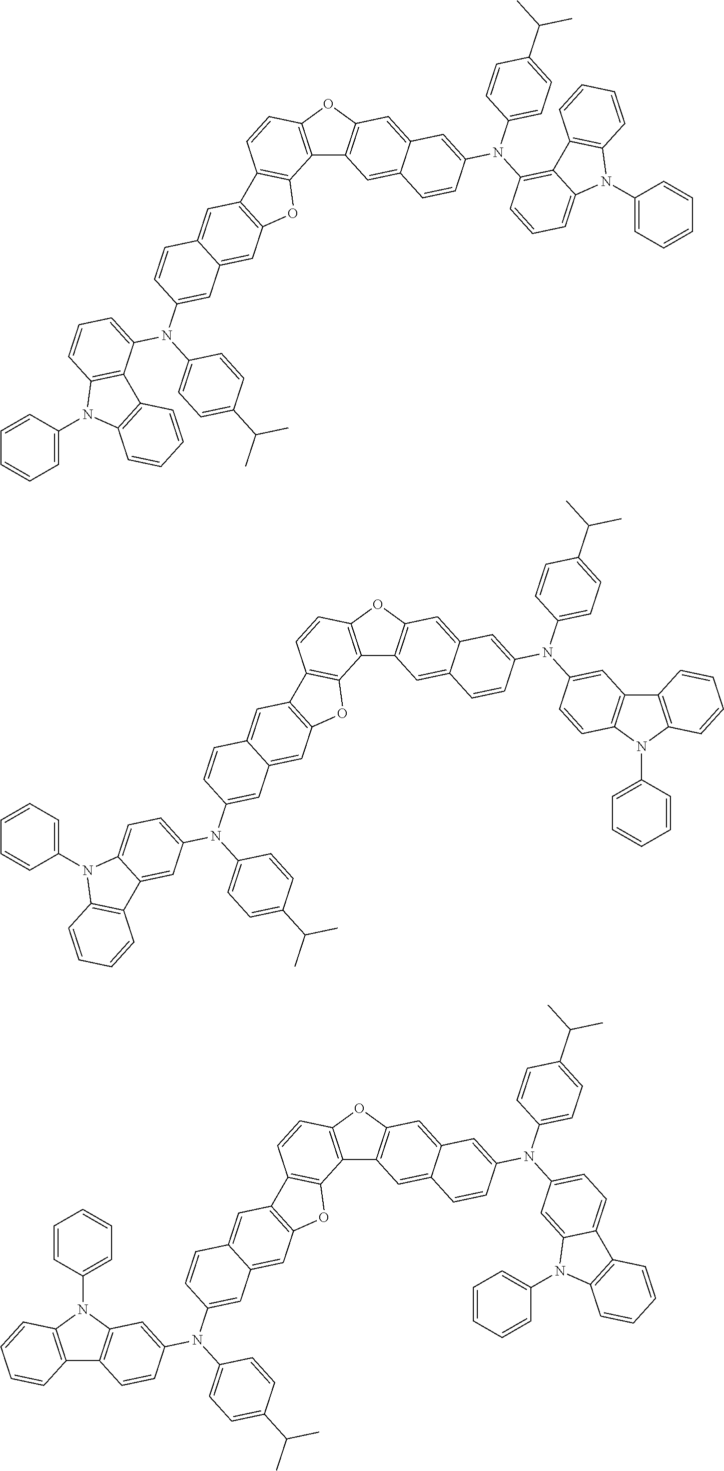 [SMILES: CC(C)c1ccc(N(c2ccc3cc4c(cc3c2)oc2c4ccc3oc4cc5cc(N(c6ccc(C(C)C)cc6)c6cccc7c6c6ccccc6n7-c6ccccc6)ccc5cc4c32)c2cccc3c2c2ccccc2n3-c2ccccc2)cc1 ; CC(C)c1ccc(N(c2ccc3cc4c(cc3c2)oc2c4ccc3oc4cc5cc(N(c6ccc(C(C)C)cc6)c6ccc7c(c6)c6ccccc6n7-c6ccccc6)ccc5cc4c32)c2ccc3c(c2)c2ccccc2n3-c2ccccc2)cc1 ; CC(C)c1ccc(N(c2ccc3cc4c(cc3c2)oc2c4ccc3oc4cc5cc(N(c6ccc(C(C)C)cc6)c6ccc7c8ccccc8n(-c8ccccc8)c7c6)ccc5cc4c32)c2ccc3c4ccccc4n(-c4ccccc4)c3c2)cc1]

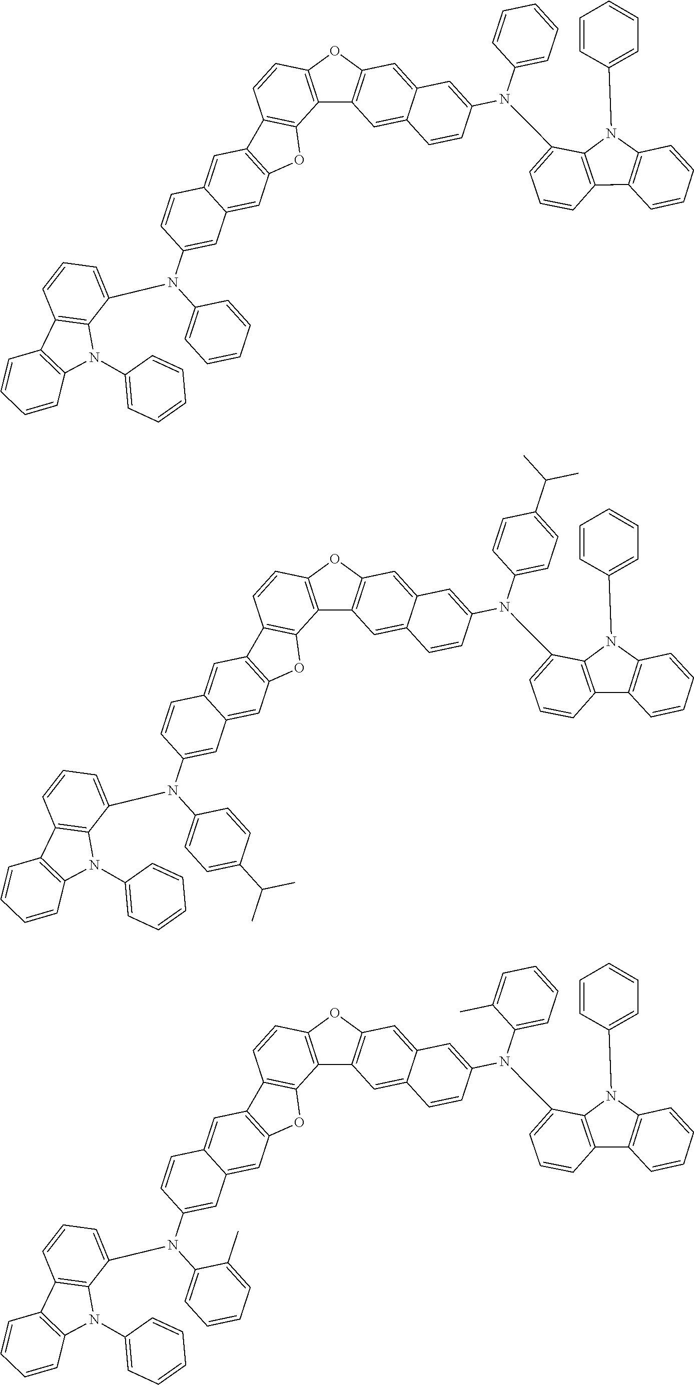 [SMILES: c1ccc(N(c2ccc3cc4c(cc3c2)oc2c4ccc3oc4cc5cc(N(c6ccccc6)c6cccc7c8ccccc8n(-c8ccccc8)c67)ccc5cc4c32)c2cccc3c4ccccc4n(-c4ccccc4)c23)cc1 ; CC(C)c1ccc(N(c2ccc3cc4c(cc3c2)oc2c4ccc3oc4cc5cc(N(c6ccc(C(C)C)cc6)c6cccc7c8ccccc8n(-c8ccccc8)c67)ccc5cc4c32)c2cccc3c4ccccc4n(-c4ccccc4)c23)cc1 ; Cc1ccccc1N(c1ccc2cc3c(cc2c1)oc1c3ccc2oc3cc4cc(N(c5ccccc5C)c5cccc6c7ccccc7n(-c7ccccc7)c56)ccc4cc3c21)c1cccc2c3ccccc3n(-c3ccccc3)c12]

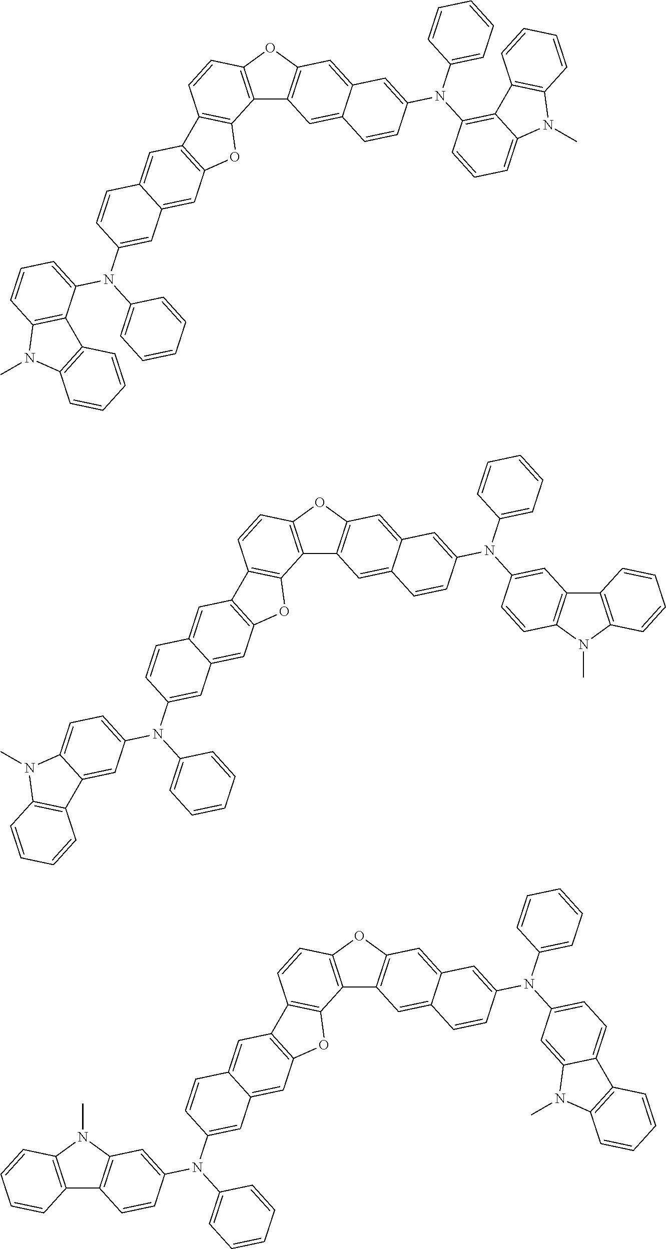

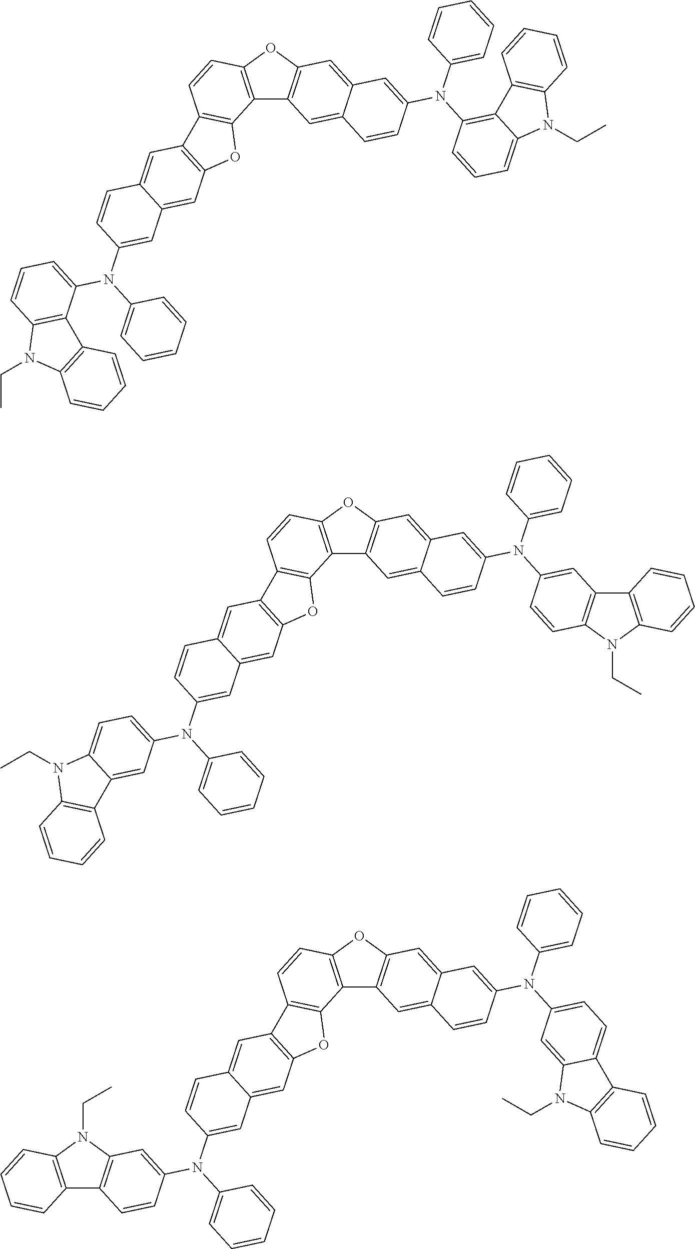

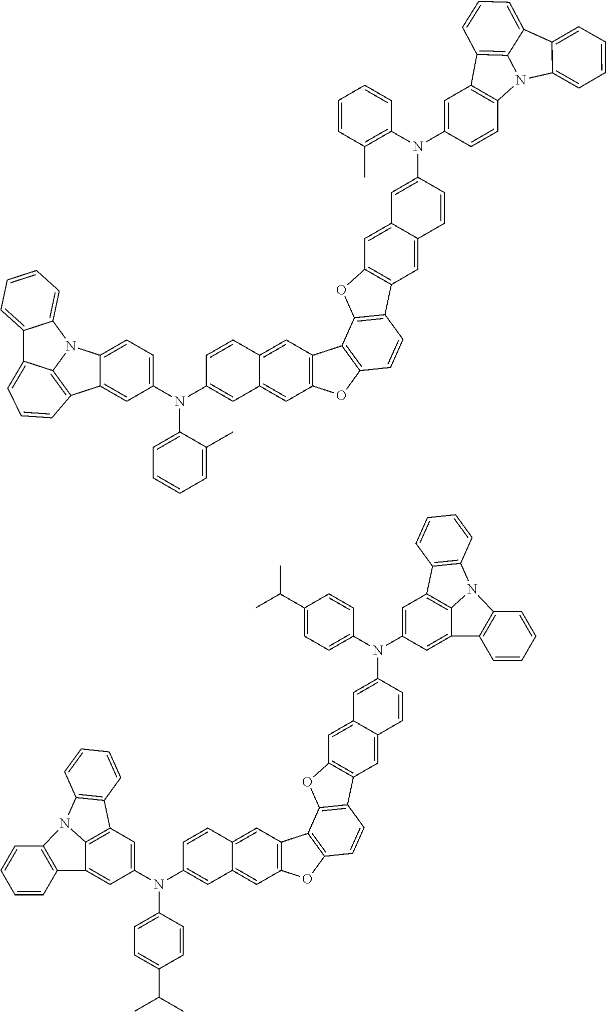 [SMILES: Cc1ccccc1N(c1ccc2cc3c(cc2c1)oc1c3ccc2oc3cc4cc(N(c5ccc6c(c5)c5cccc7c8ccccc8n6c75)c5ccccc5C)ccc4cc3c21)c1ccc2c(c1)c1cccc3c4ccccc4n2c31 ; CC(C)c1ccc(N(c2ccc3cc4c(cc3c2)oc2c4ccc3oc4cc5cc(N(c6ccc(C(C)C)cc6)c6cc7c8ccccc8n8c9ccccc9c(c6)c78)ccc5cc4c32)c2cc3c4ccccc4n4c5ccccc5c(c2)c34)cc1]

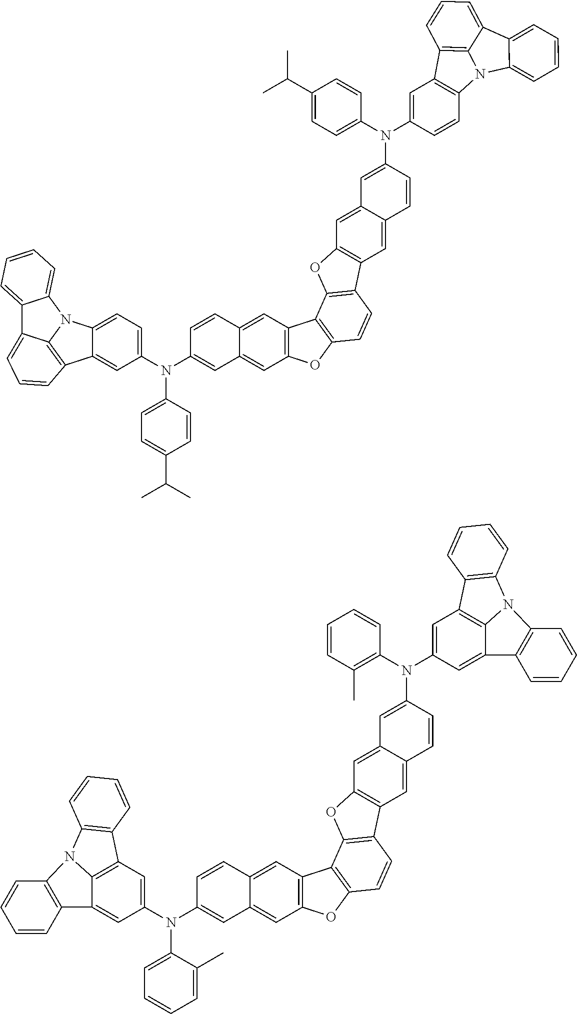 [SMILES: CC(C)c1ccc(N(c2ccc3cc4c(cc3c2)oc2c4ccc3oc4cc5cc(N(c6ccc(C(C)C)cc6)c6ccc7c(c6)c6cccc8c9ccccc9n7c86)ccc5cc4c32)c2ccc3c(c2)c2cccc4c5ccccc5n3c42)cc1 ; Cc1ccccc1N(c1ccc2cc3c(cc2c1)oc1c3ccc2oc3cc4cc(N(c5cc6c7ccccc7n7c8ccccc8c(c5)c67)c5ccccc5C)ccc4cc3c21)c1cc2c3ccccc3n3c4ccccc4c(c1)c23]

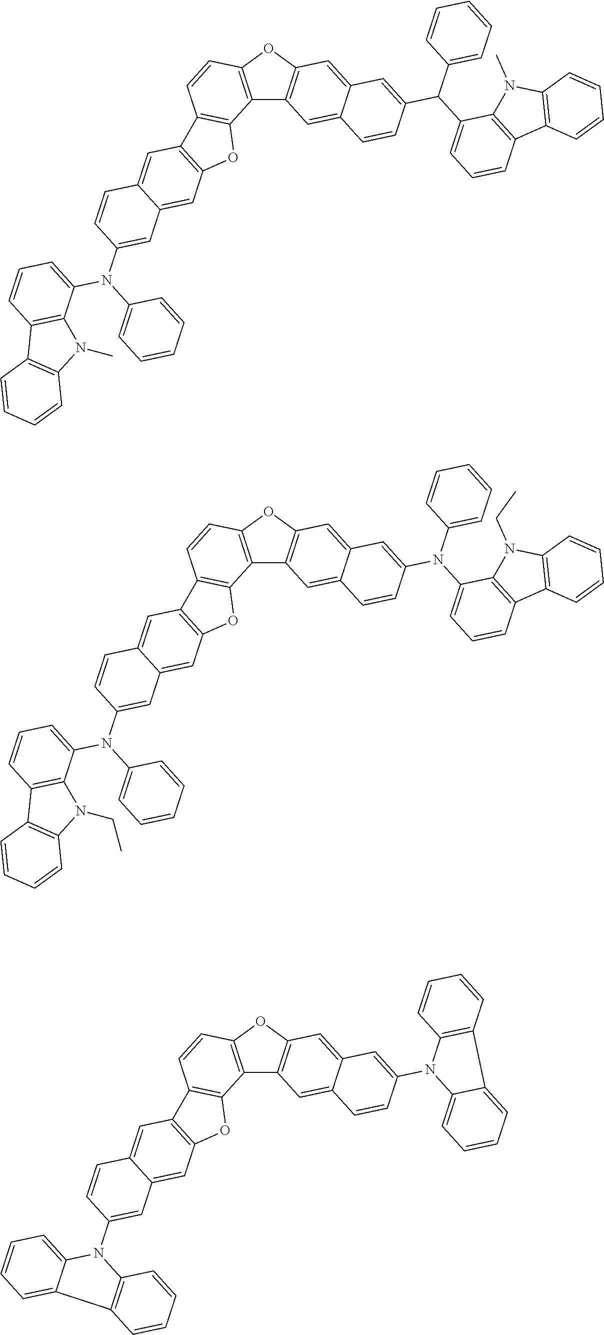 [SMILES: Cn1c2ccccc2c2cccc(C(c3ccccc3)c3ccc4cc5c(cc4c3)oc3ccc4c6cc7ccc(N(c8ccccc8)c8cccc9c%10ccccc%10n(C)c89)cc7cc6oc4c35)c21 ; CCn1c2ccccc2c2cccc(N(c3ccccc3)c3ccc4cc5c(cc4c3)oc3c5ccc4oc5cc6cc(N(c7ccccc7)c7cccc8c9ccccc9n(CC)c78)ccc6cc5c43)c21 ; c1ccc2c(c1)c1ccccc1n2-c1ccc2cc3c(cc2c1)oc1c3ccc2oc3cc4cc(-n5c6ccccc6c6ccccc65)ccc4cc3c21]

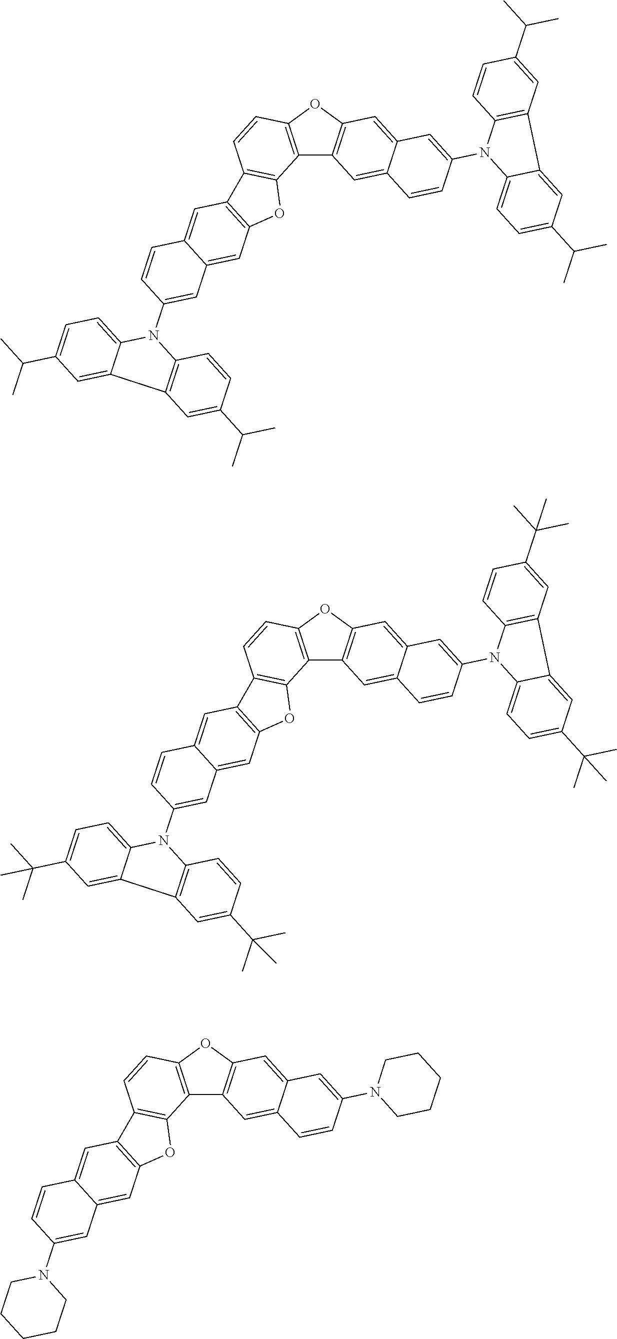

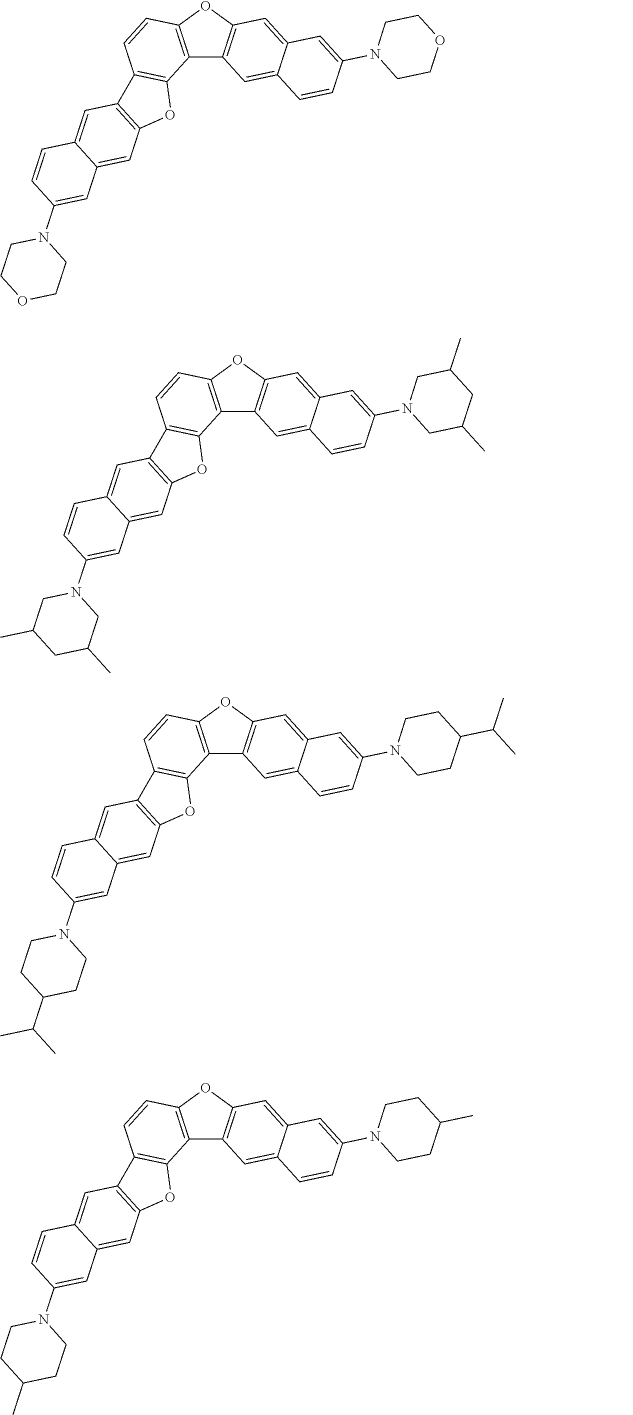

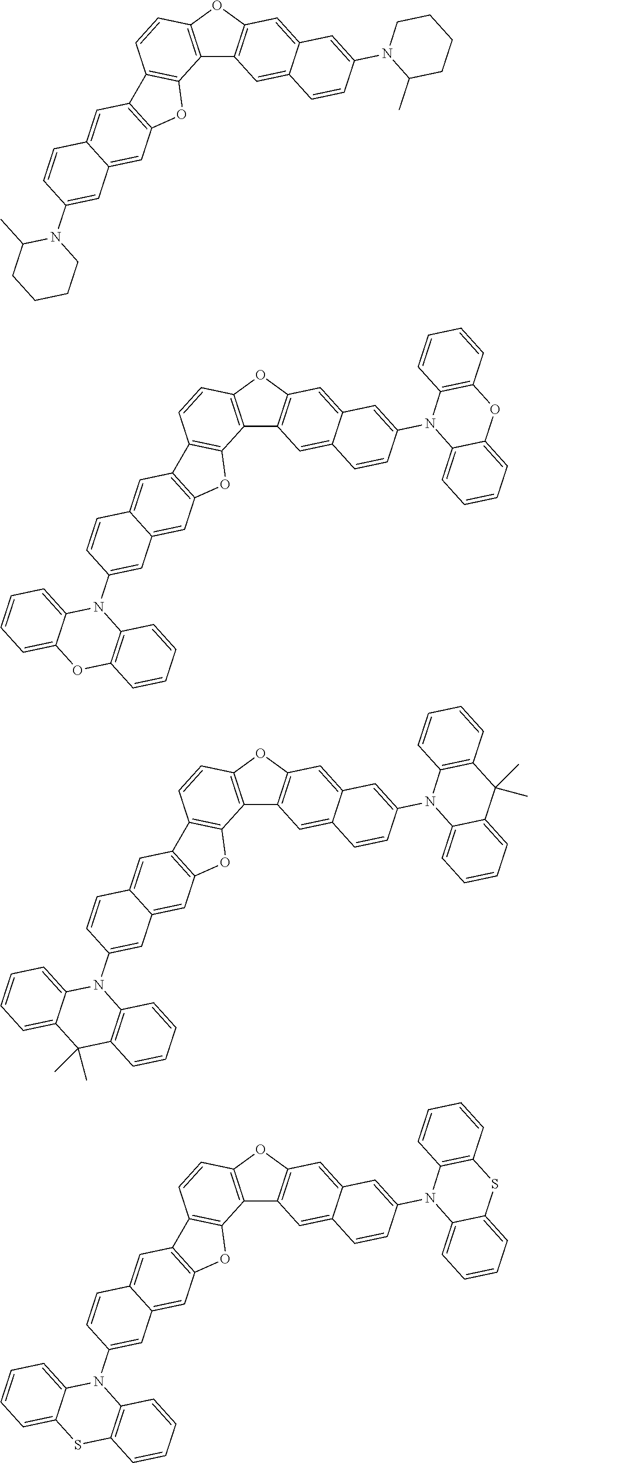 [SMILES: CC1CCCCN1c1ccc2cc3c(cc2c1)oc1c3ccc2oc3cc4cc(N5CCCCC5C)ccc4cc3c21 ; c1ccc2c(c1)Oc1ccccc1N2c1ccc2cc3c(cc2c1)oc1c3ccc2oc3cc4cc(N5c6ccccc6Oc6ccccc65)ccc4cc3c21 ; CC1(C)c2ccccc2N(c2ccc3cc4c(cc3c2)oc2c4ccc3oc4cc5cc(N6c7ccccc7C(C)(C)c7ccccc76)ccc5cc4c32)c2ccccc21 ; c1ccc2c(c1)Sc1ccccc1N2c1ccc2cc3c(cc2c1)oc1c3ccc2oc3cc4cc(N5c6ccccc6Sc6ccccc65)ccc4cc3c21]

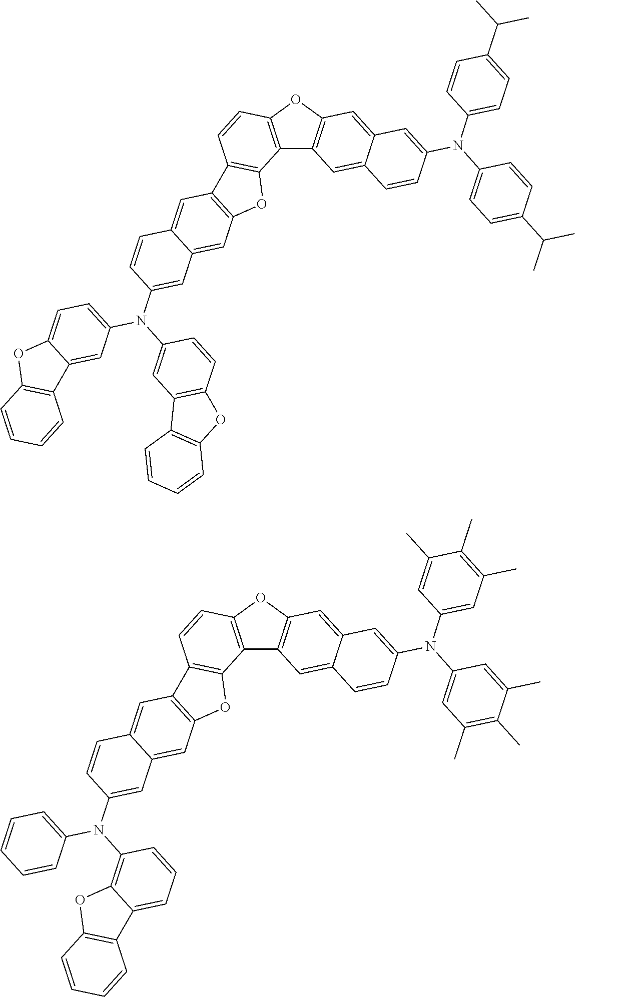 [SMILES: CC(C)c1ccc(N(c2ccc(C(C)C)cc2)c2ccc3cc4c(cc3c2)oc2ccc3c5cc6ccc(N(c7ccc8oc9ccccc9c8c7)c7ccc8oc9ccccc9c8c7)cc6cc5oc3c24)cc1 ; Cc1cc(N(c2cc(C)c(C)c(C)c2)c2ccc3cc4c(cc3c2)oc2ccc3c5cc6ccc(N(c7ccccc7)c7cccc8c7oc7ccccc78)cc6cc5oc3c24)cc(C)c1C]

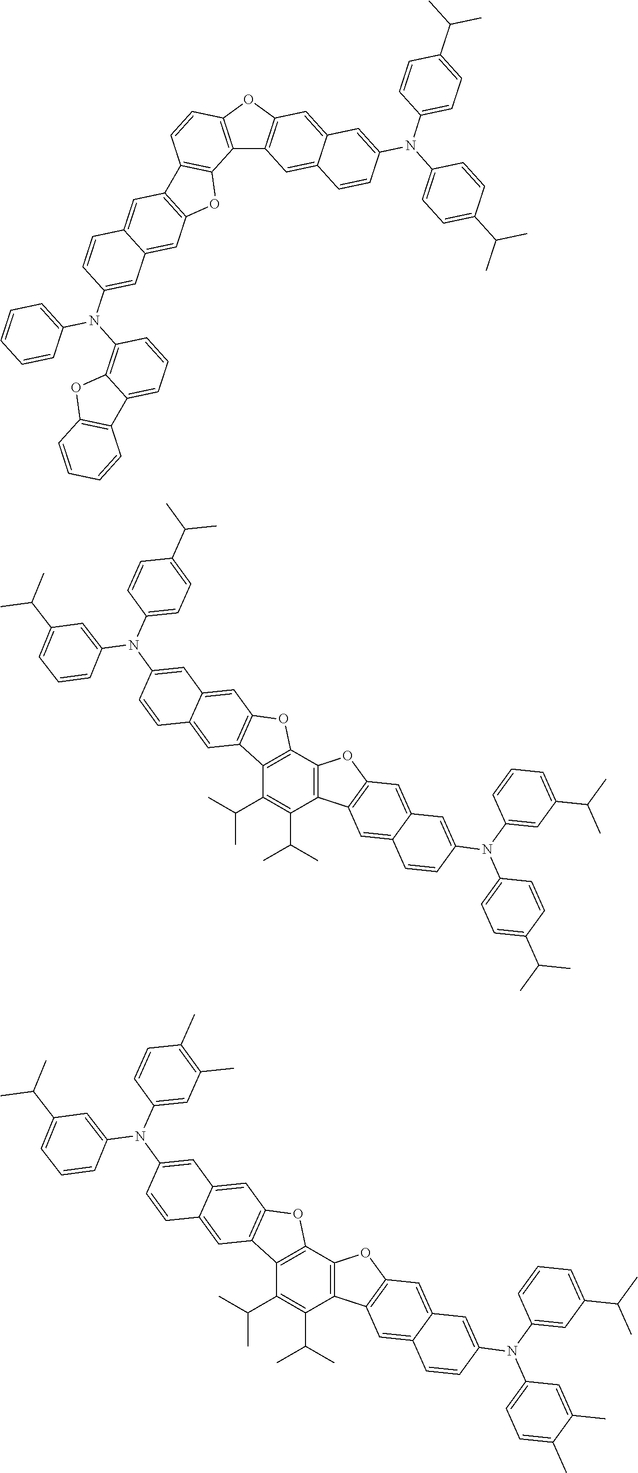

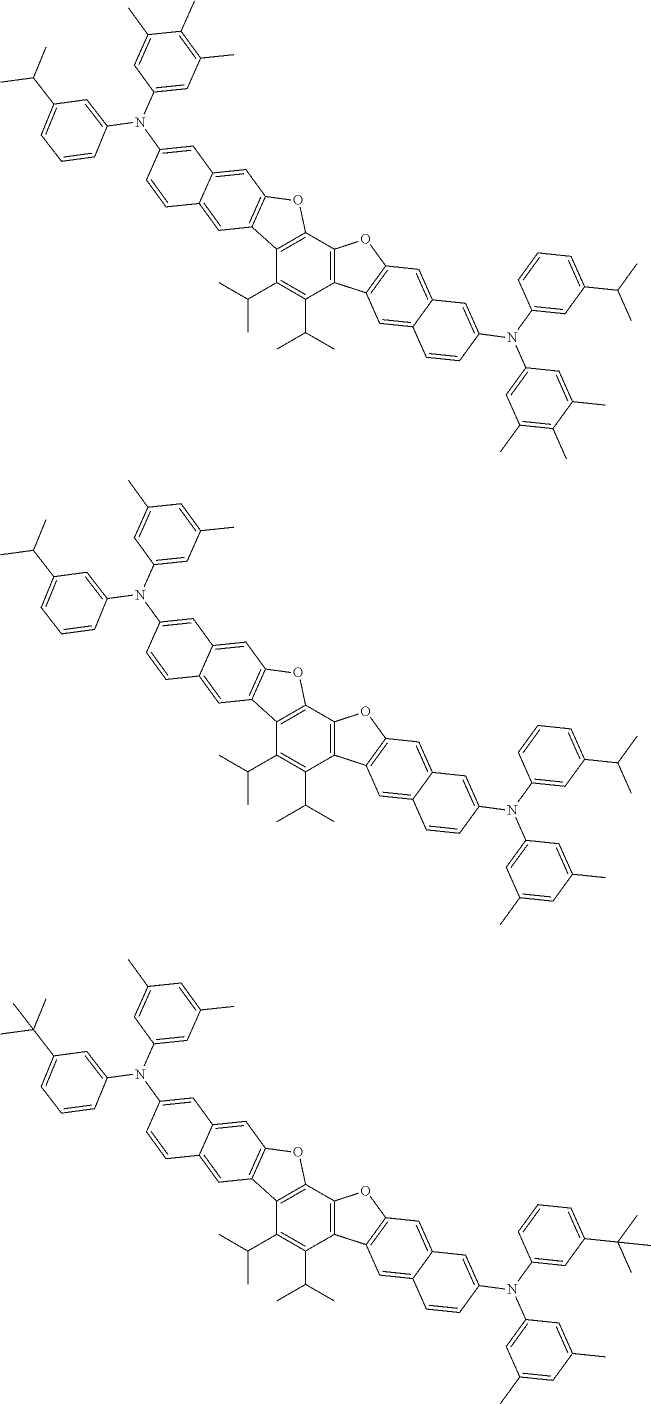

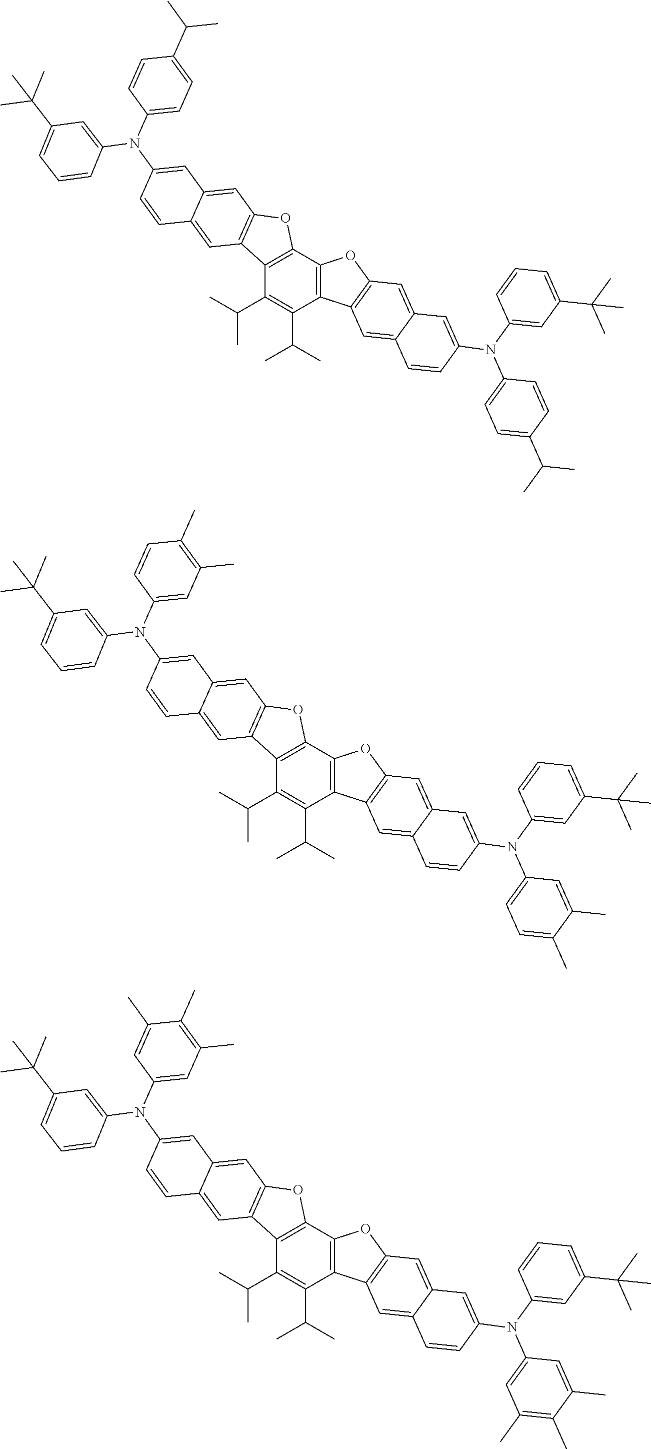

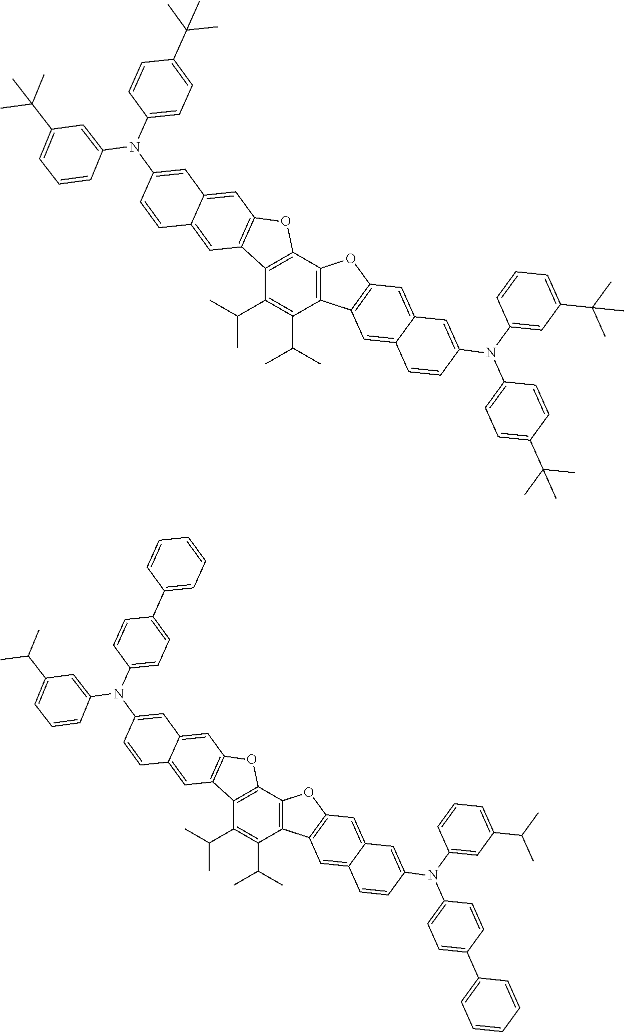

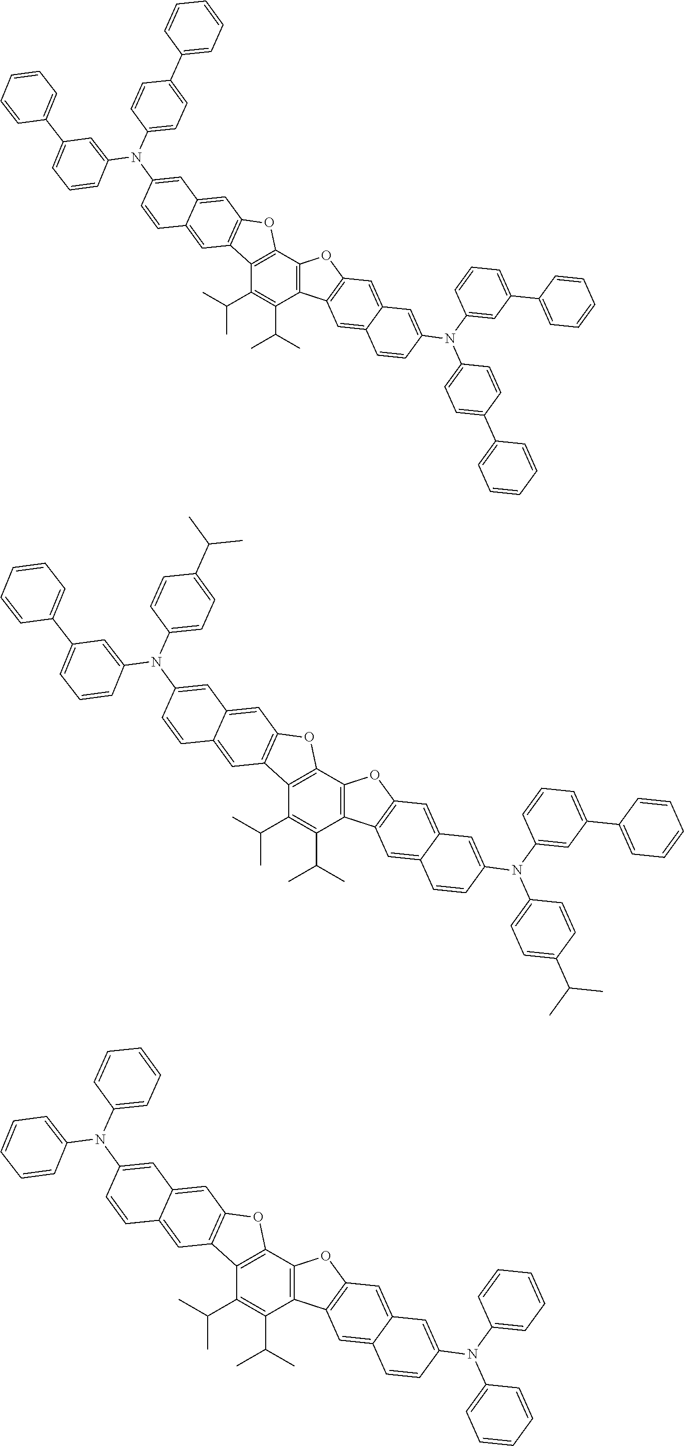 [SMILES: CC(C)c1c(C(C)C)c2c3cc4ccc(N(c5ccc(-c6ccccc6)cc5)c5cccc(-c6ccccc6)c5)cc4cc3oc2c2oc3cc4cc(N(c5ccc(-c6ccccc6)cc5)c5cccc(-c6ccccc6)c5)ccc4cc3c12 ; CC(C)c1ccc(N(c2cccc(-c3ccccc3)c2)c2ccc3cc4c(cc3c2)oc2c3oc5cc6cc(N(c7ccc(C(C)C)cc7)c7cccc(-c8ccccc8)c7)ccc6cc5c3c(C(C)C)c(C(C)C)c42)cc1 ; CC(C)c1c(C(C)C)c2c3cc4ccc(N(c5ccccc5)c5ccccc5)cc4cc3oc2c2oc3cc4cc(N(c5ccccc5)c5ccccc5)ccc4cc3c12]

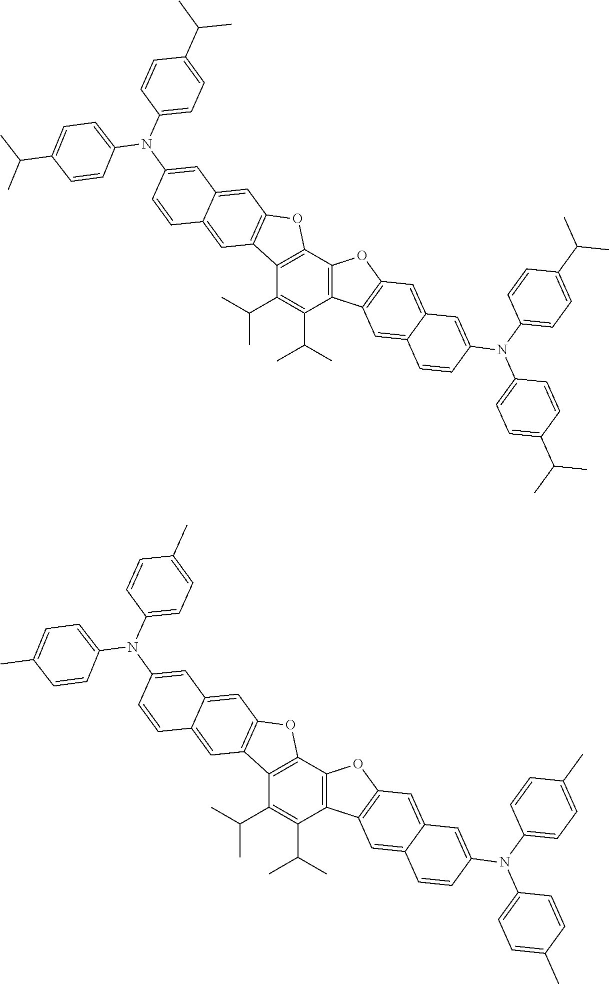

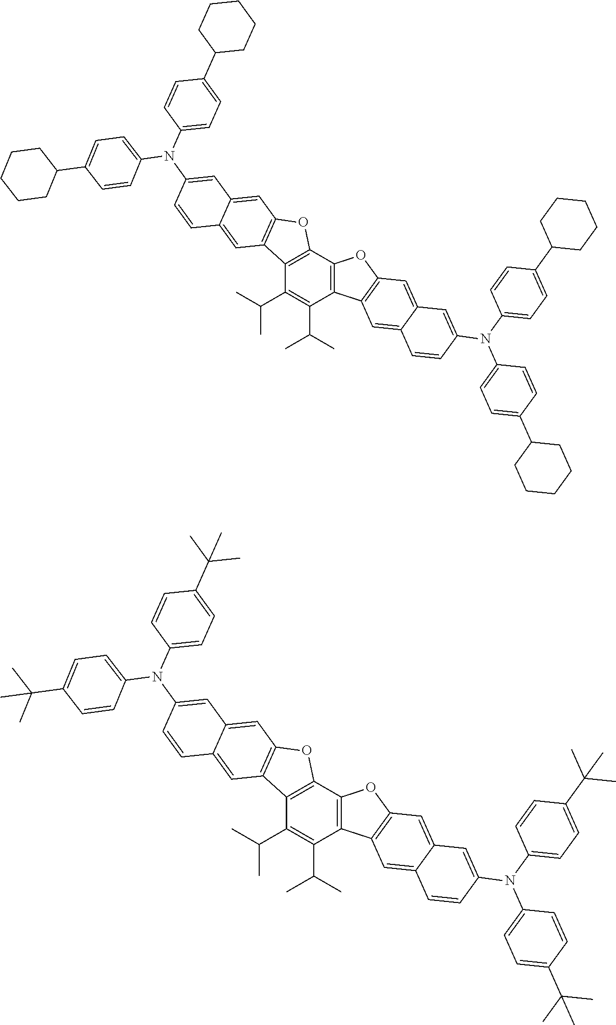 [SMILES: CC(C)c1c(C(C)C)c2c3cc4ccc(N(c5ccc(C6CCCCC6)cc5)c5ccc(C6CCCCC6)cc5)cc4cc3oc2c2oc3cc4cc(N(c5ccc(C6CCCCC6)cc5)c5ccc(C6CCCCC6)cc5)ccc4cc3c12 ; CC(C)c1c(C(C)C)c2c3cc4ccc(N(c5ccc(C(C)(C)C)cc5)c5ccc(C(C)(C)C)cc5)cc4cc3oc2c2oc3cc4cc(N(c5ccc(C(C)(C)C)cc5)c5ccc(C(C)(C)C)cc5)ccc4cc3c12]

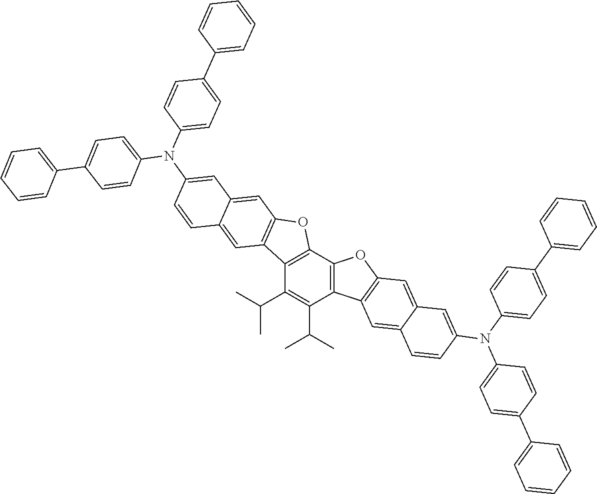 [SMILES: CC(C)c1c(C(C)C)c2c3cc4ccc(N(c5ccc(-c6ccccc6)cc5)c5ccc(-c6ccccc6)cc5)cc4cc3oc2c2oc3cc4cc(N(c5ccc(-c6ccccc6)cc5)c5ccc(-c6ccccc6)cc5)ccc4cc3c12]

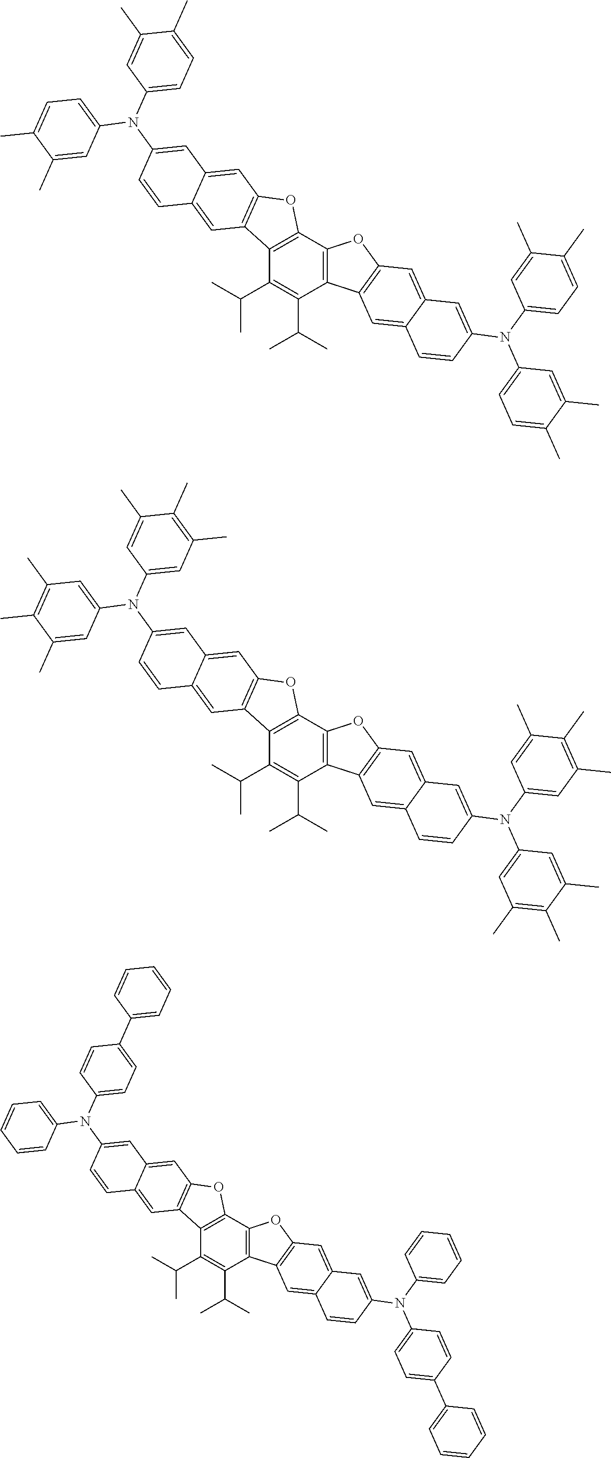

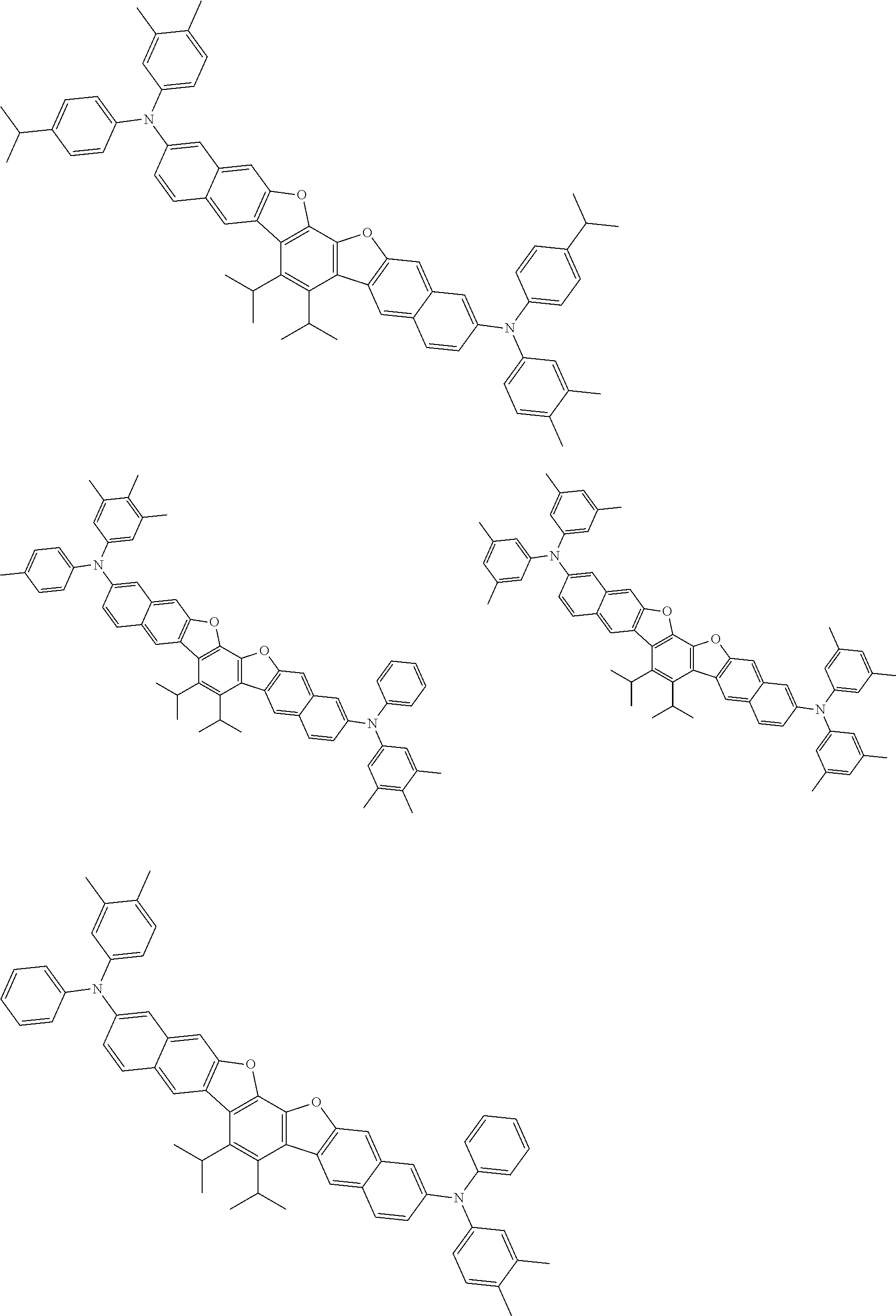

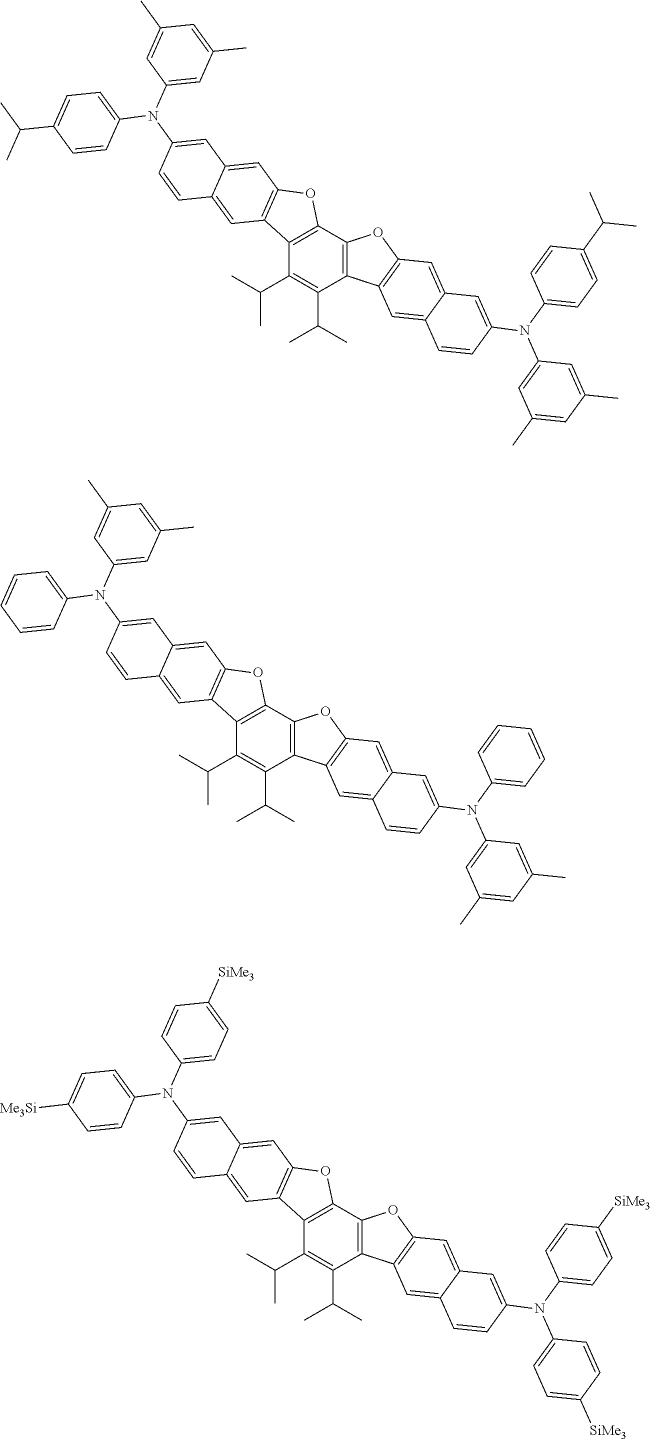 [SMILES: Cc1cc(C)cc(N(c2ccc(C(C)C)cc2)c2ccc3cc4c(cc3c2)oc2c3oc5cc6cc(N(c7ccc(C(C)C)cc7)c7cc(C)cc(C)c7)ccc6cc5c3c(C(C)C)c(C(C)C)c42)c1 ; Cc1cc(C)cc(N(c2ccccc2)c2ccc3cc4c(cc3c2)oc2c3oc5cc6cc(N(c7ccccc7)c7cc(C)cc(C)c7)ccc6cc5c3c(C(C)C)c(C(C)C)c42)c1 ; CC(C)c1c(C(C)C)c2c3cc4ccc(N(c5ccc([Si](C)(C)C)cc5)c5ccc([Si](C)(C)C)cc5)cc4cc3oc2c2oc3cc4cc(N(c5ccc([Si](C)(C)C)cc5)c5ccc([Si](C)(C)C)cc5)ccc4cc3c12]

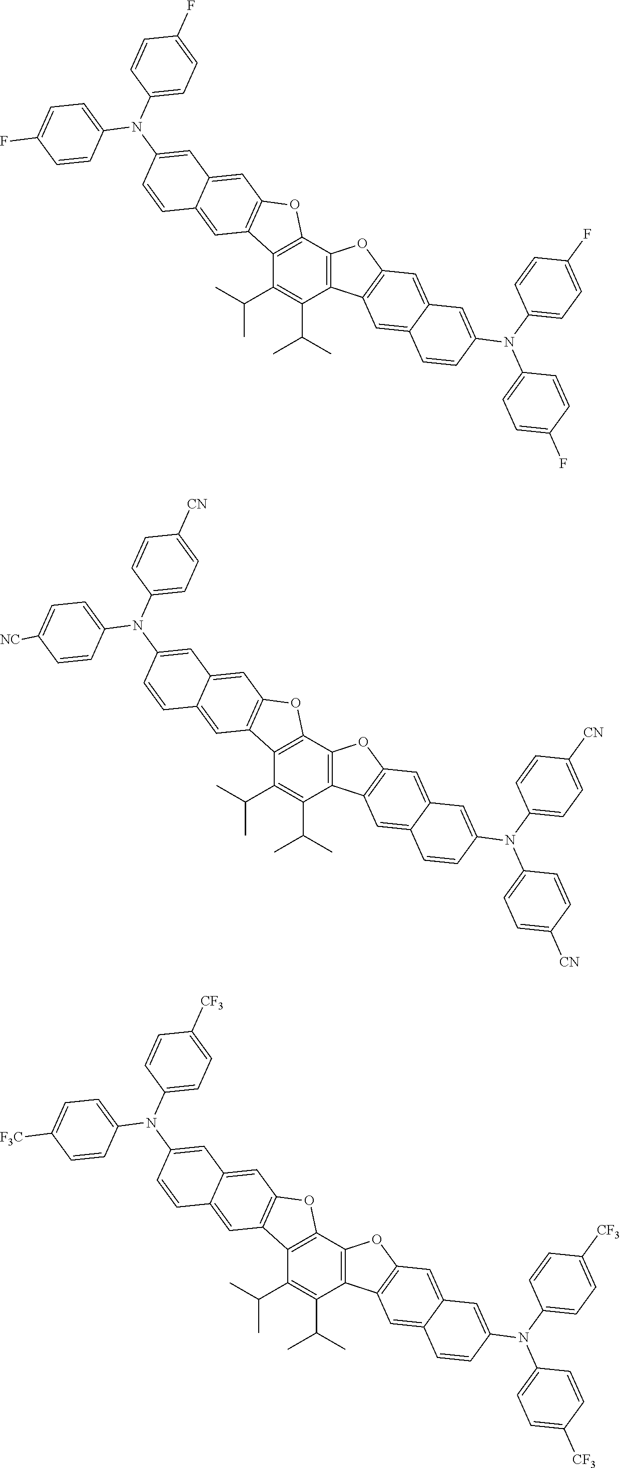

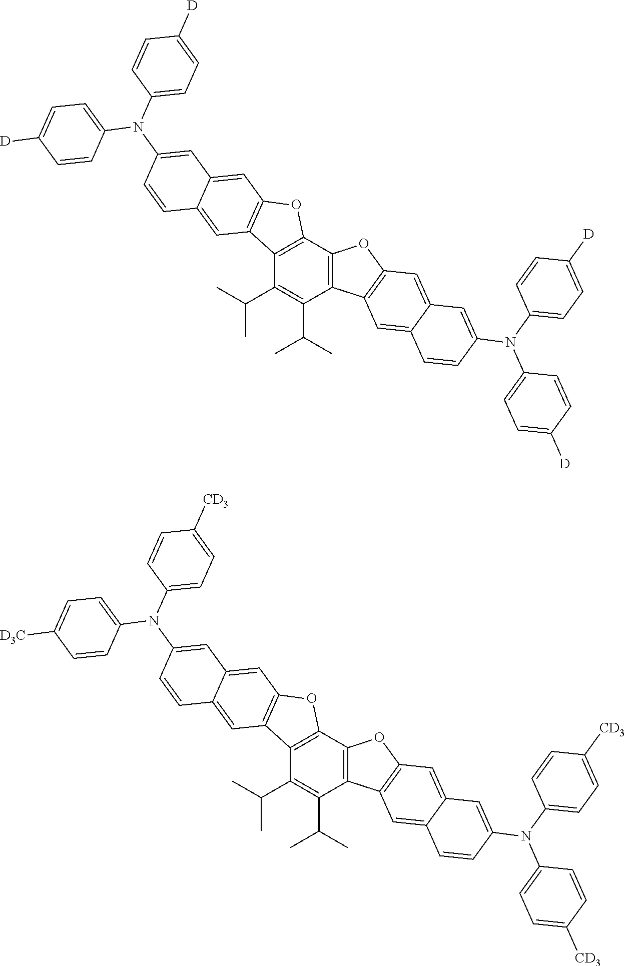 [SMILES: [2H]c1ccc(N(c2ccc([2H])cc2)c2ccc3cc4c(cc3c2)oc2c3oc5cc6cc(N(c7ccc([2H])cc7)c7ccc([2H])cc7)ccc6cc5c3c(C(C)C)c(C(C)C)c42)cc1 ; [2H]C([2H])([2H])c1ccc(N(c2ccc(C([2H])([2H])[2H])cc2)c2ccc3cc4c(cc3c2)oc2c3oc5cc6cc(N(c7ccc(C([2H])([2H])[2H])cc7)c7ccc(C([2H])([2H])[2H])cc7)ccc6cc5c3c(C(C)C)c(C(C)C)c42)cc1]

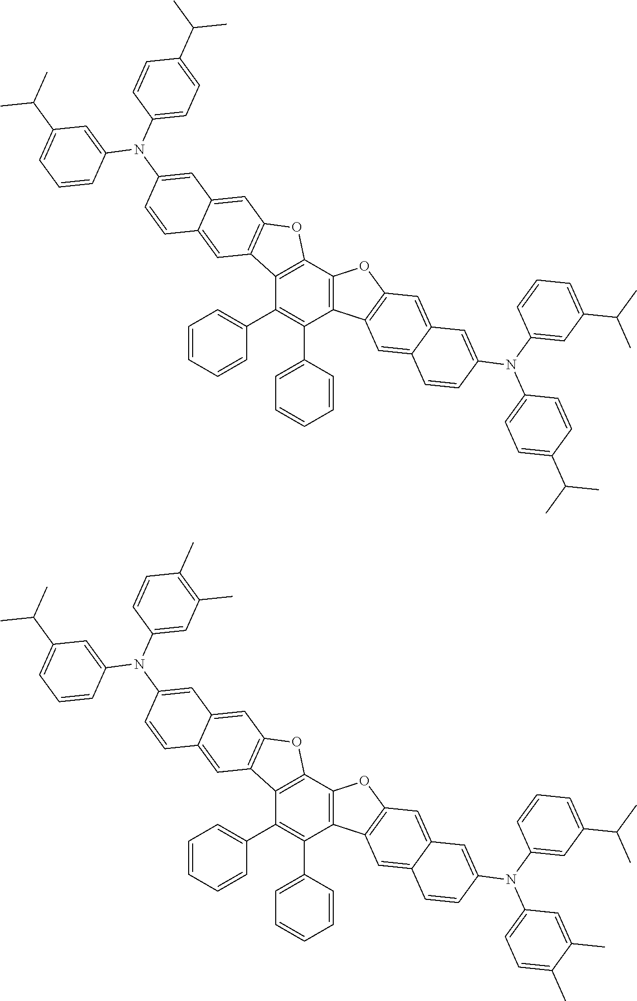

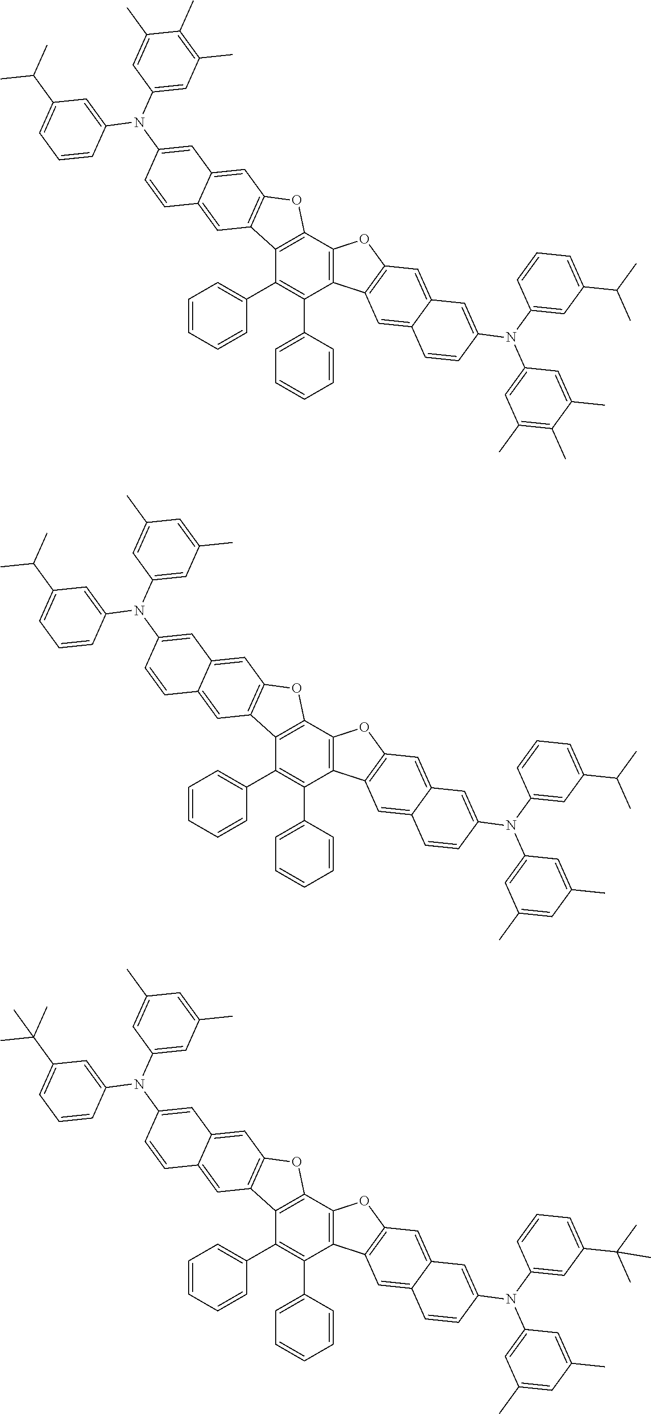

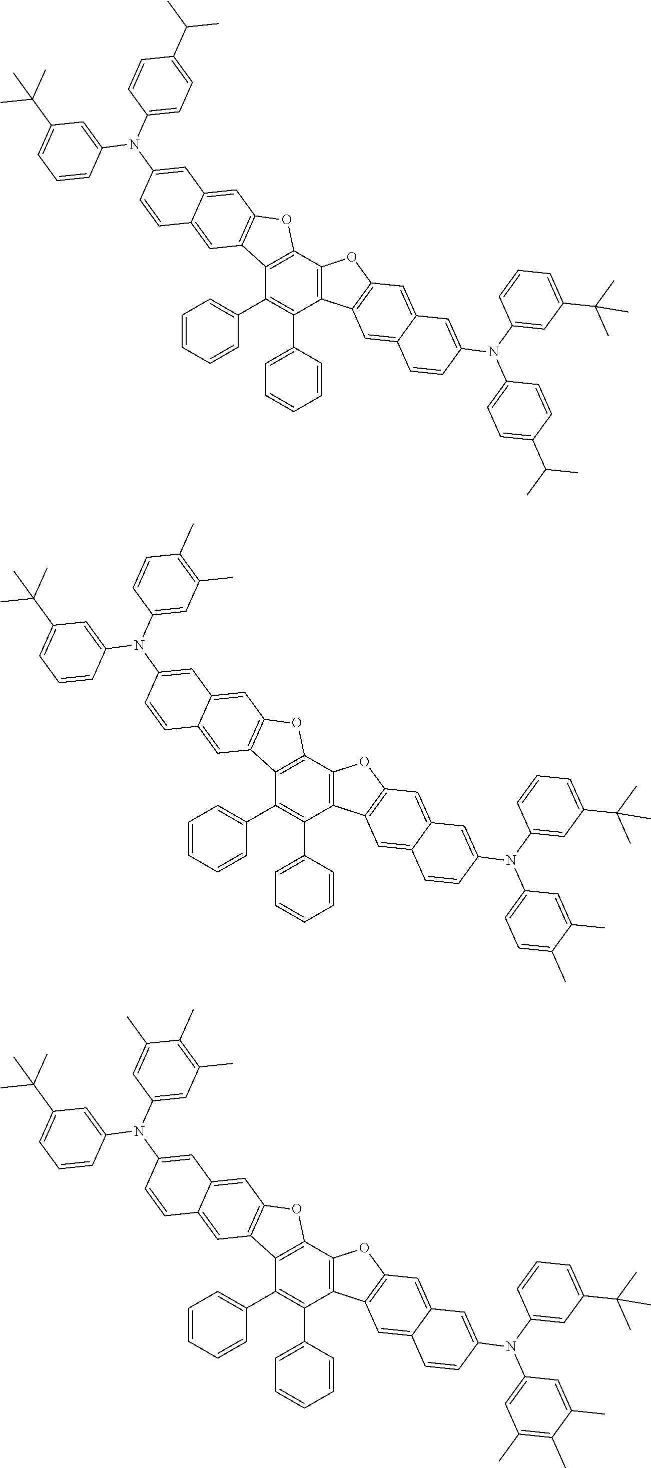

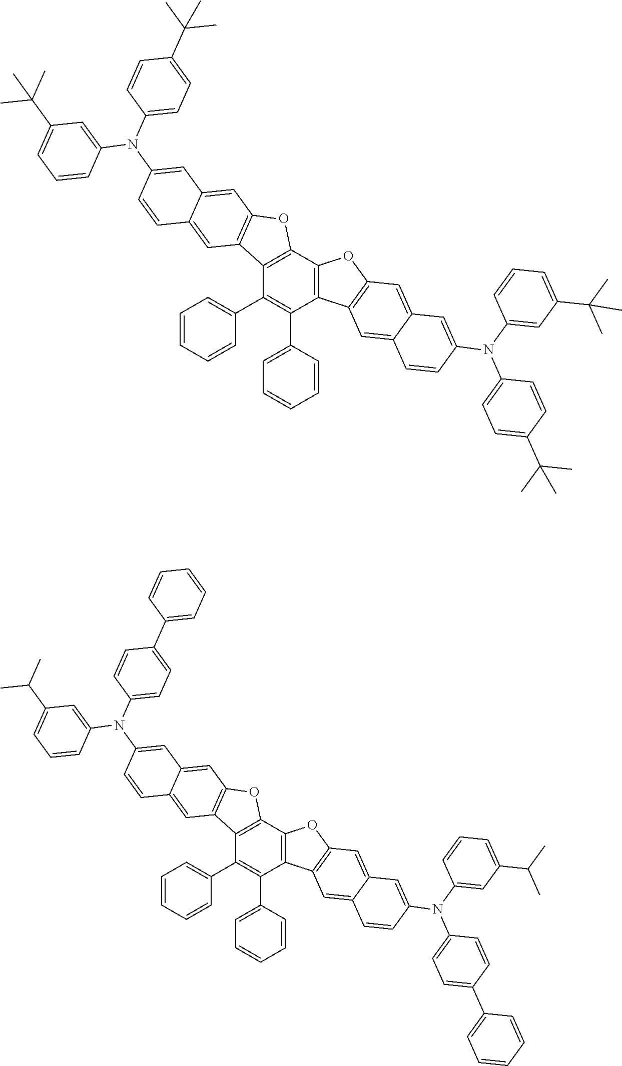

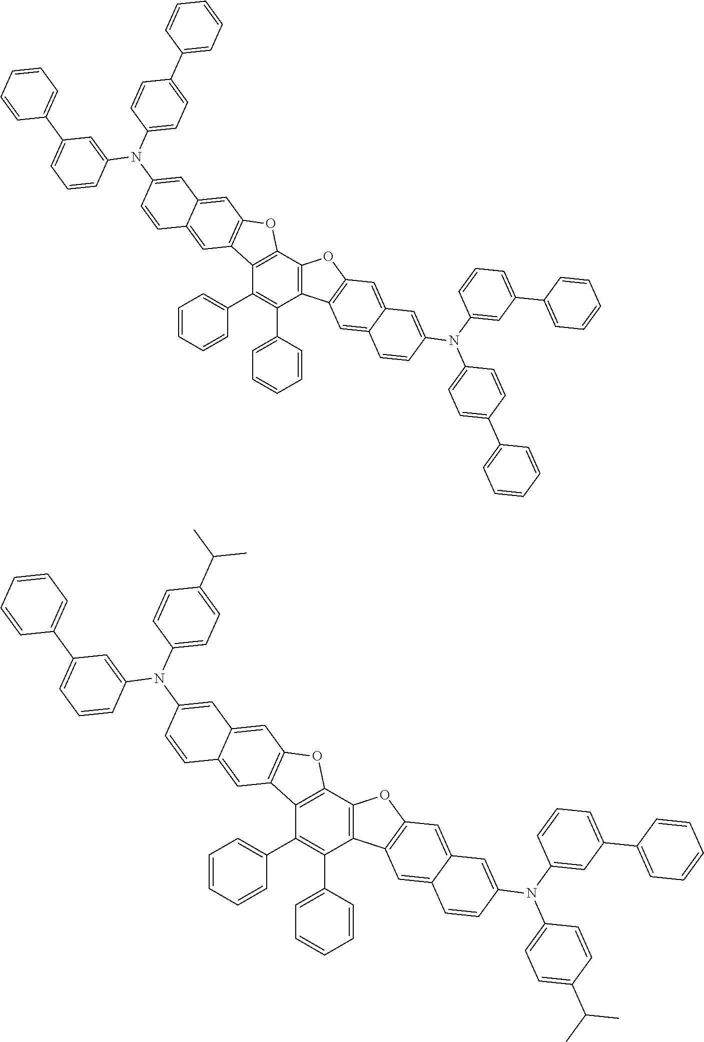 [SMILES: c1ccc(-c2ccc(N(c3cccc(-c4ccccc4)c3)c3ccc4cc5c(cc4c3)oc3c4oc6cc7cc(N(c8ccc(-c9ccccc9)cc8)c8cccc(-c9ccccc9)c8)ccc7cc6c4c(-c4ccccc4)c(-c4ccccc4)c53)cc2)cc1 ; CC(C)c1ccc(N(c2cccc(-c3ccccc3)c2)c2ccc3cc4c(cc3c2)oc2c3oc5cc6cc(N(c7ccc(C(C)C)cc7)c7cccc(-c8ccccc8)c7)ccc6cc5c3c(-c3ccccc3)c(-c3ccccc3)c42)cc1]

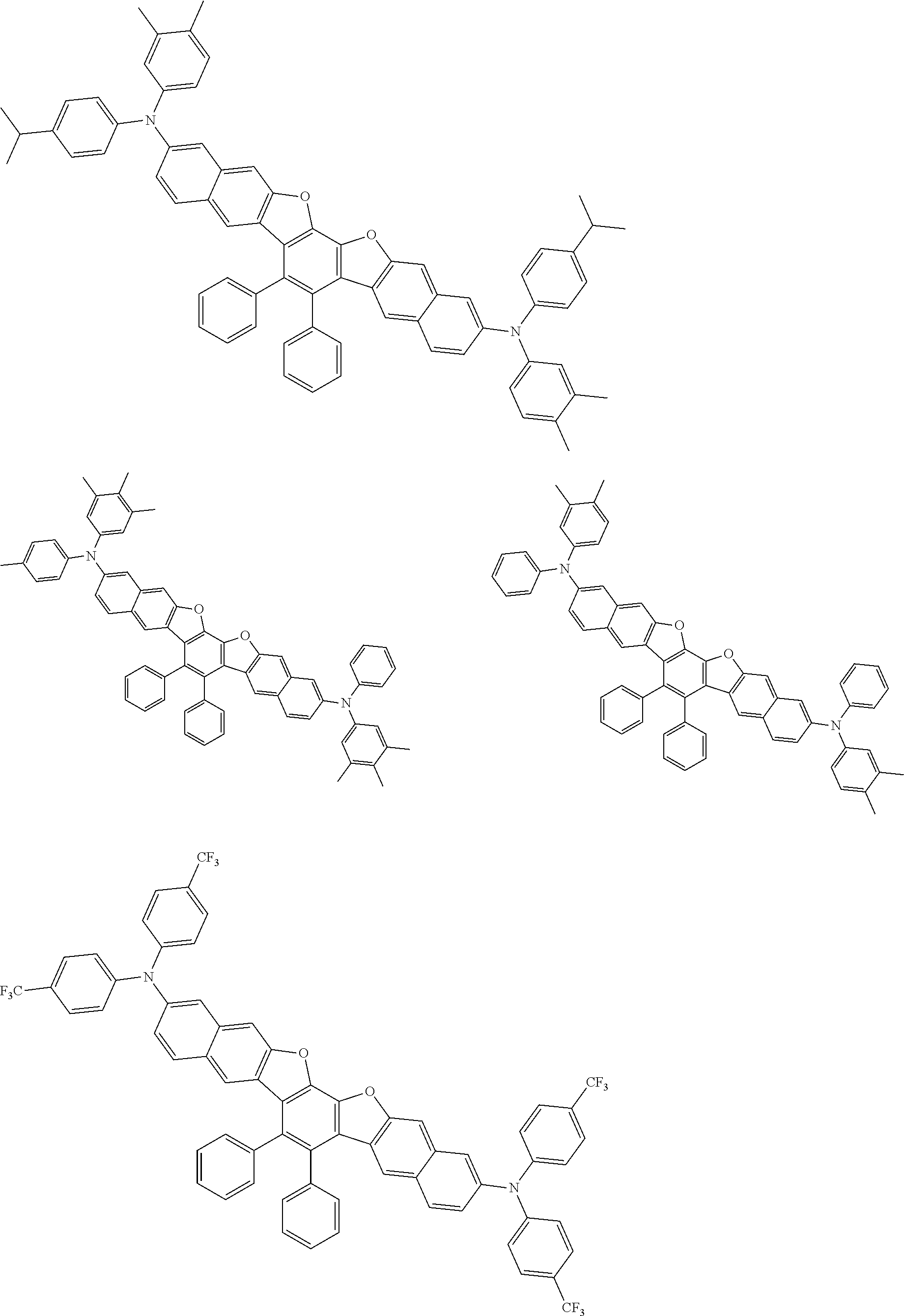

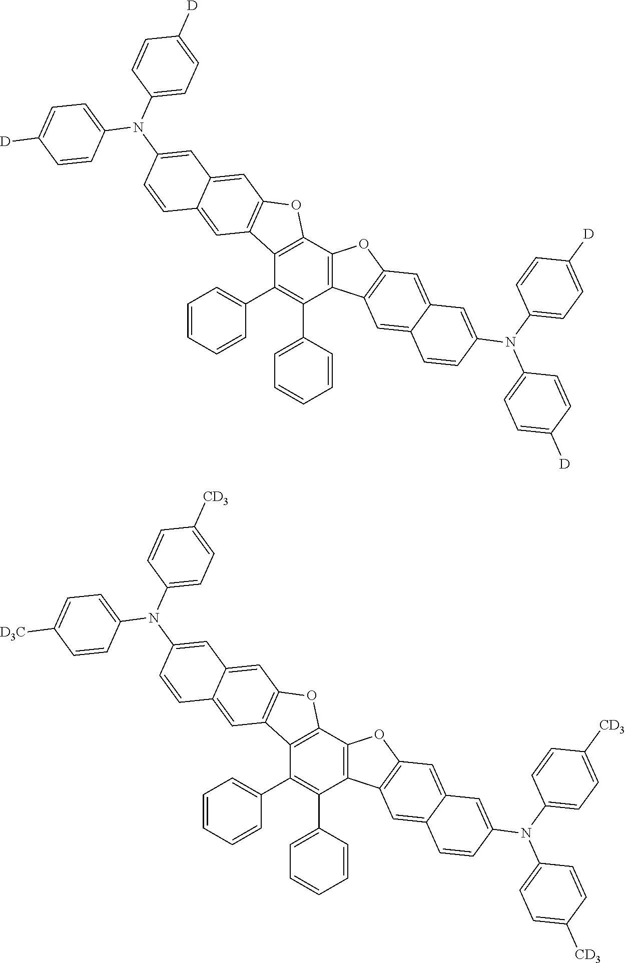 [SMILES: [2H]c1ccc(N(c2ccc([2H])cc2)c2ccc3cc4c(cc3c2)oc2c3oc5cc6cc(N(c7ccc([2H])cc7)c7ccc([2H])cc7)ccc6cc5c3c(-c3ccccc3)c(-c3ccccc3)c42)cc1 ; [2H]C([2H])([2H])c1ccc(N(c2ccc(C([2H])([2H])[2H])cc2)c2ccc3cc4c(cc3c2)oc2c3oc5cc6cc(N(c7ccc(C([2H])([2H])[2H])cc7)c7ccc(C([2H])([2H])[2H])cc7)ccc6cc5c3c(-c3ccccc3)c(-c3ccccc3)c42)cc1]

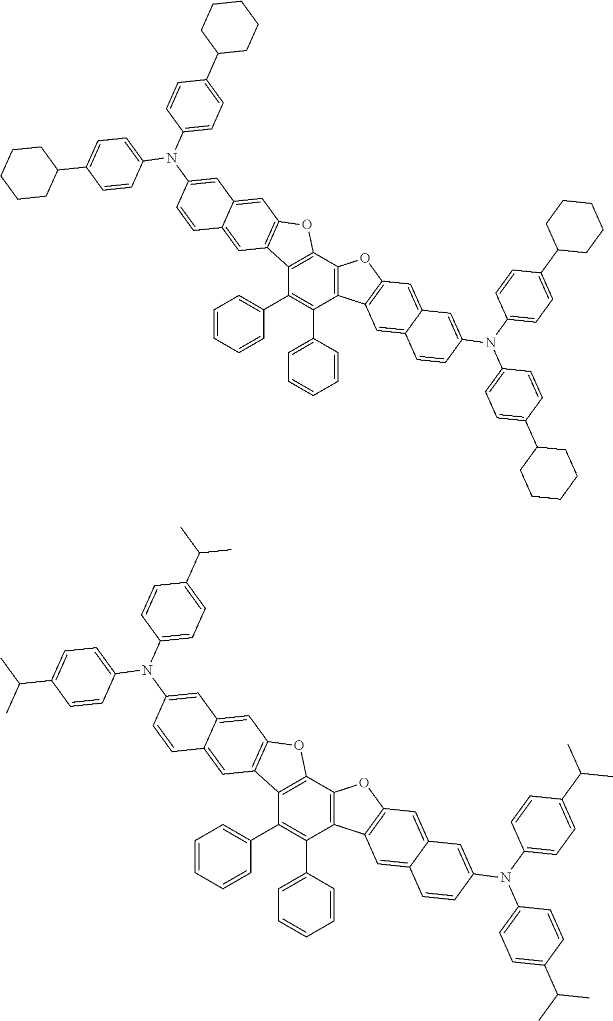 [SMILES: c1ccc(-c2c(-c3ccccc3)c3c4cc5ccc(N(c6ccc(C7CCCCC7)cc6)c6ccc(C7CCCCC7)cc6)cc5cc4oc3c3oc4cc5cc(N(c6ccc(C7CCCCC7)cc6)c6ccc(C7CCCCC7)cc6)ccc5cc4c23)cc1 ; CC(C)c1ccc(N(c2ccc(C(C)C)cc2)c2ccc3cc4c(cc3c2)oc2c3oc5cc6cc(N(c7ccc(C(C)C)cc7)c7ccc(C(C)C)cc7)ccc6cc5c3c(-c3ccccc3)c(-c3ccccc3)c42)cc1]

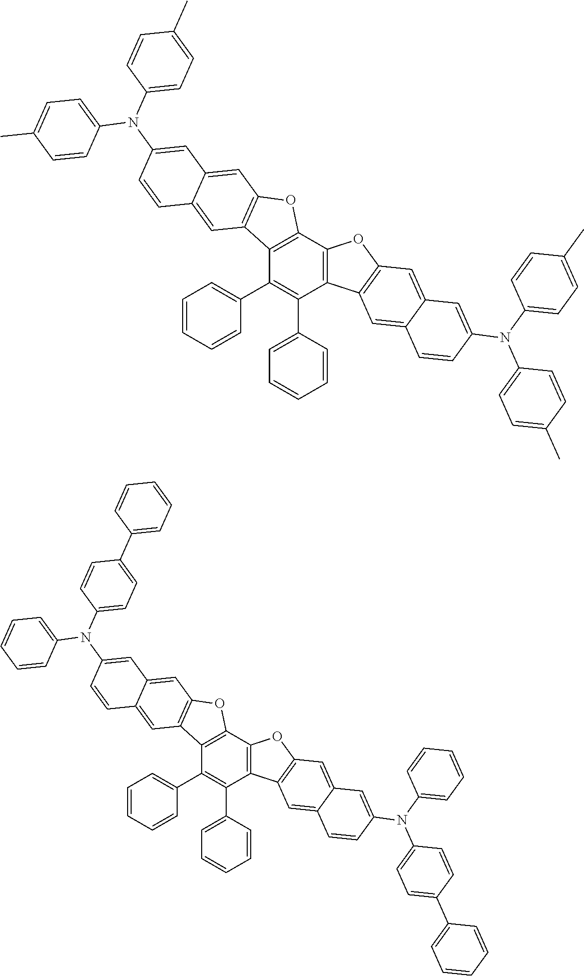

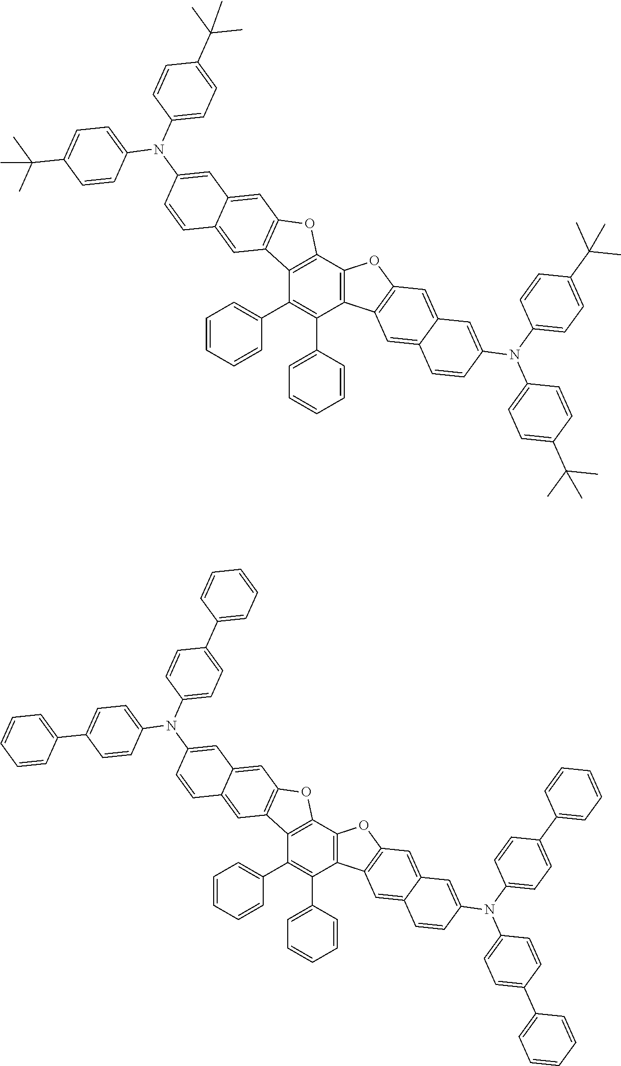 [SMILES: CC(C)(C)c1ccc(N(c2ccc(C(C)(C)C)cc2)c2ccc3cc4c(cc3c2)oc2c3oc5cc6cc(N(c7ccc(C(C)(C)C)cc7)c7ccc(C(C)(C)C)cc7)ccc6cc5c3c(-c3ccccc3)c(-c3ccccc3)c42)cc1 ; c1ccc(-c2ccc(N(c3ccc(-c4ccccc4)cc3)c3ccc4cc5c(cc4c3)oc3c4oc6cc7cc(N(c8ccc(-c9ccccc9)cc8)c8ccc(-c9ccccc9)cc8)ccc7cc6c4c(-c4ccccc4)c(-c4ccccc4)c53)cc2)cc1]

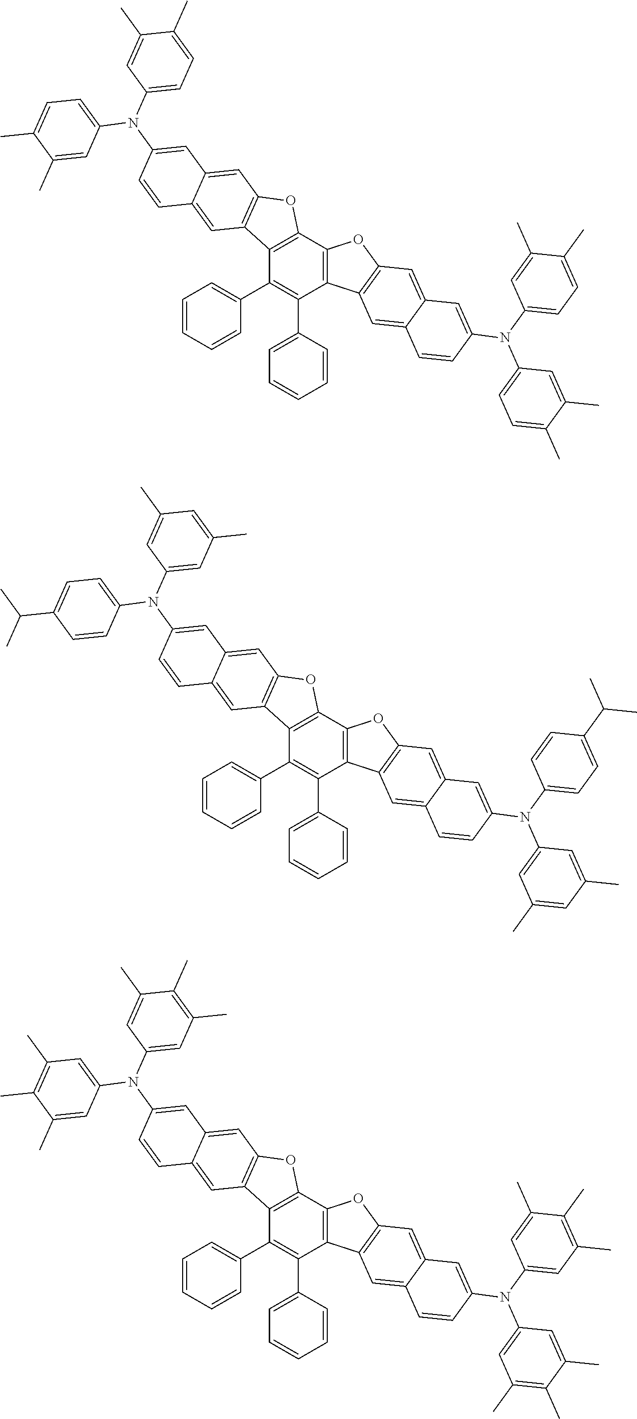

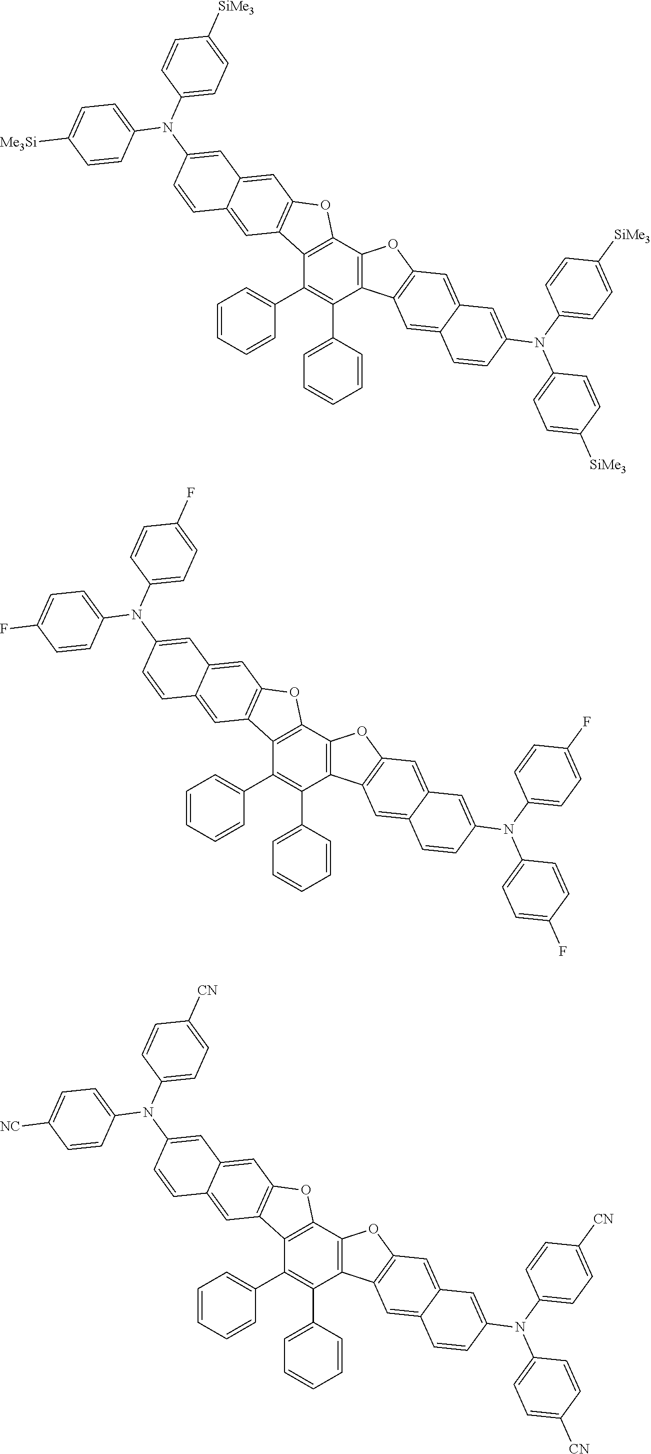 [SMILES: C[Si](C)(C)c1ccc(N(c2ccc([Si](C)(C)C)cc2)c2ccc3cc4c(cc3c2)oc2c3oc5cc6cc(N(c7ccc([Si](C)(C)C)cc7)c7ccc([Si](C)(C)C)cc7)ccc6cc5c3c(-c3ccccc3)c(-c3ccccc3)c42)cc1 ; Fc1ccc(N(c2ccc(F)cc2)c2ccc3cc4c(cc3c2)oc2c3oc5cc6cc(N(c7ccc(F)cc7)c7ccc(F)cc7)ccc6cc5c3c(-c3ccccc3)c(-c3ccccc3)c42)cc1 ; N#Cc1ccc(N(c2ccc(C#N)cc2)c2ccc3cc4c(cc3c2)oc2c3oc5cc6cc(N(c7ccc(C#N)cc7)c7ccc(C#N)cc7)ccc6cc5c3c(-c3ccccc3)c(-c3ccccc3)c42)cc1]

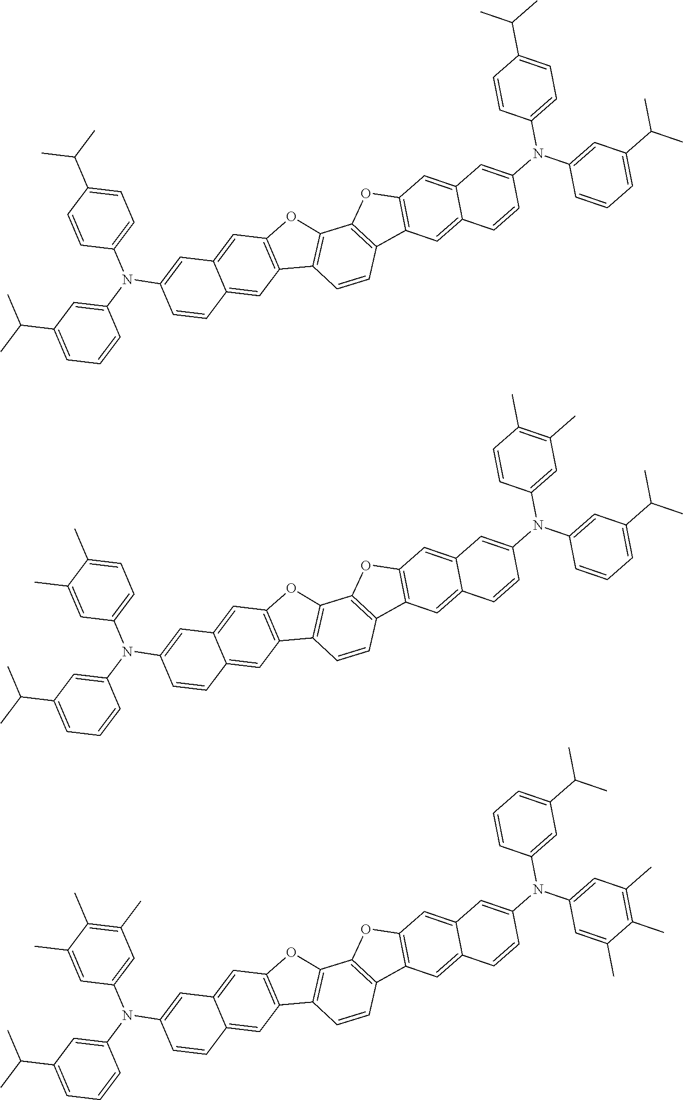

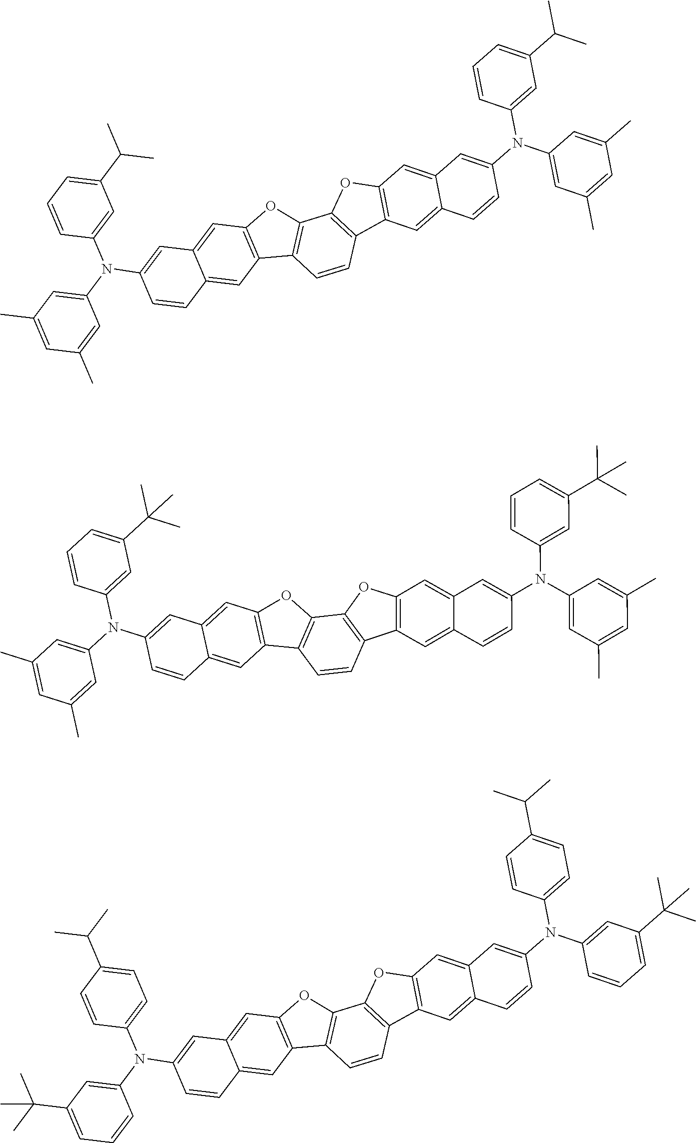

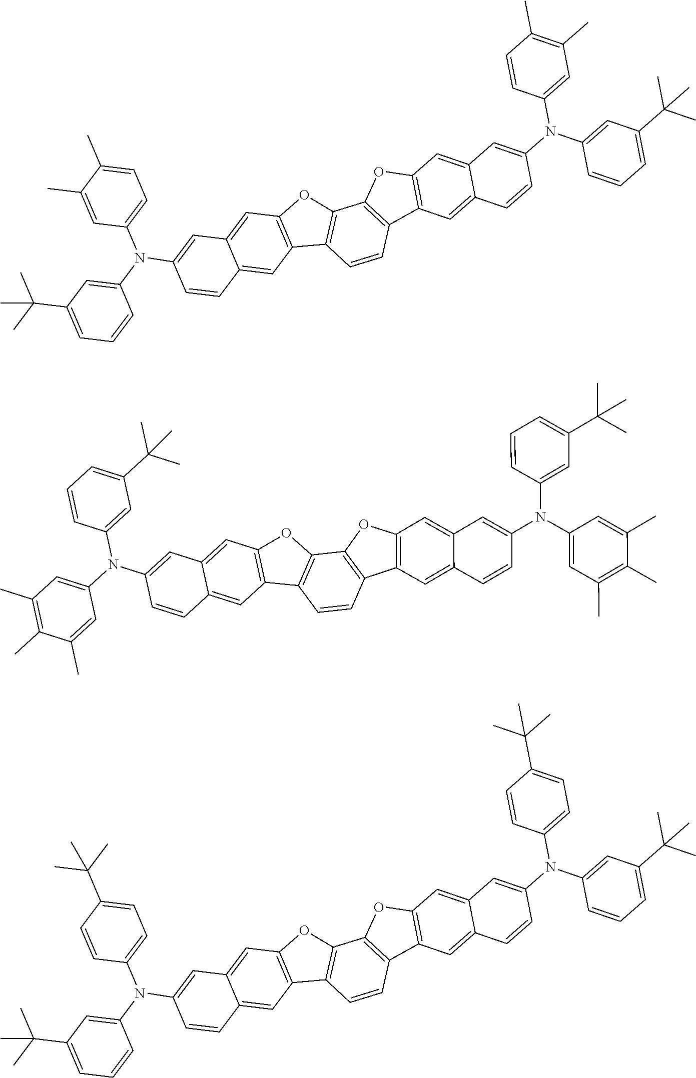

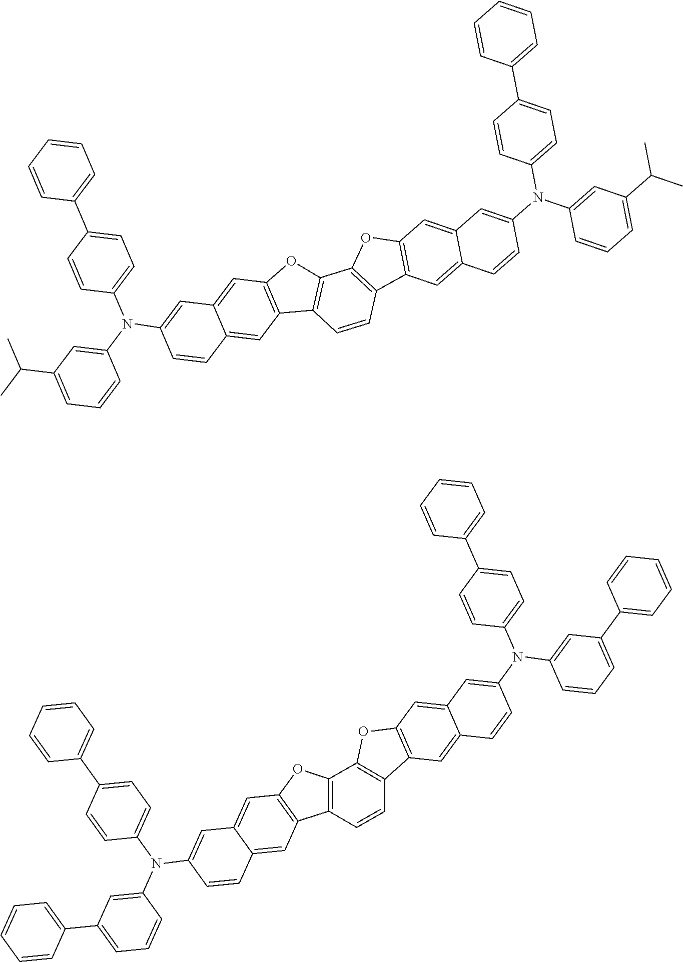 [SMILES: CC(C)c1cccc(N(c2ccc(-c3ccccc3)cc2)c2ccc3cc4c(cc3c2)oc2c4ccc3c4cc5ccc(N(c6ccc(-c7ccccc7)cc6)c6cccc(C(C)C)c6)cc5cc4oc32)c1 ; c1ccc(-c2ccc(N(c3cccc(-c4ccccc4)c3)c3ccc4cc5c(cc4c3)oc3c5ccc4c5cc6ccc(N(c7ccc(-c8ccccc8)cc7)c7cccc(-c8ccccc8)c7)cc6cc5oc43)cc2)cc1]

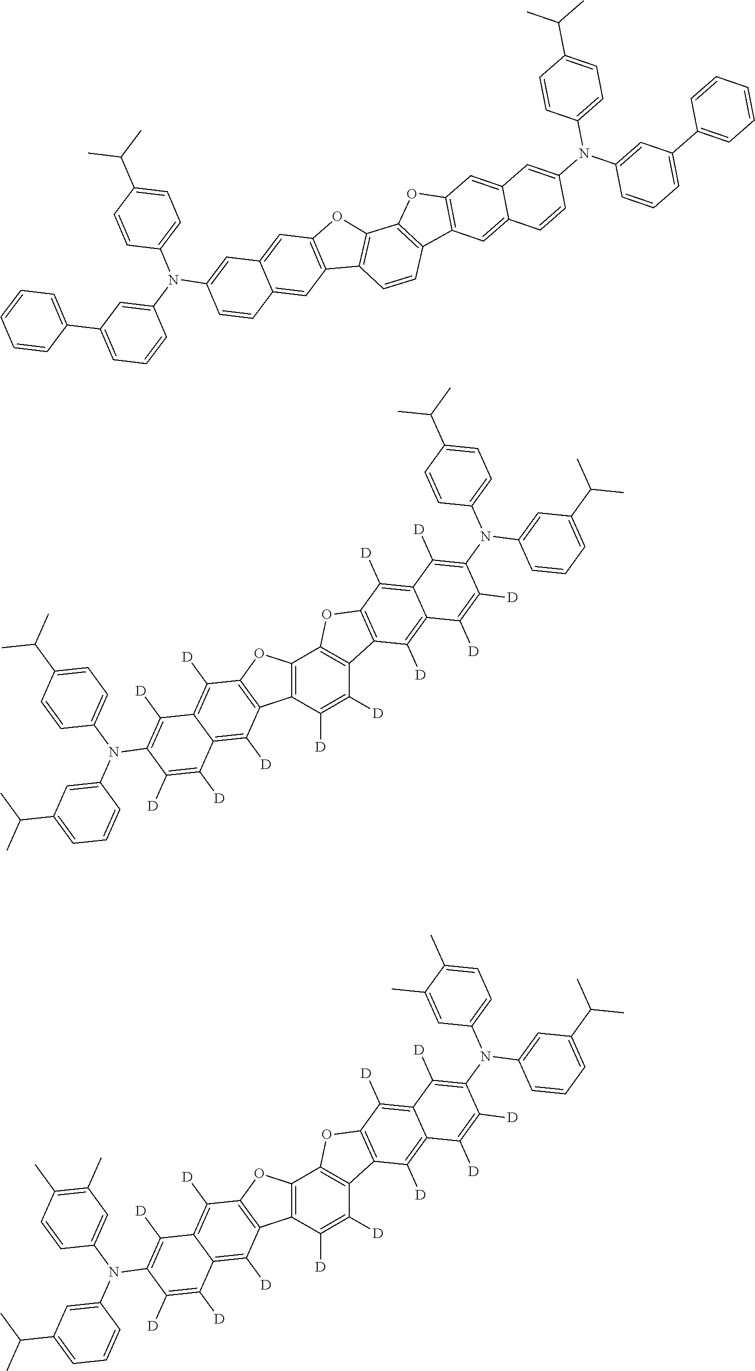

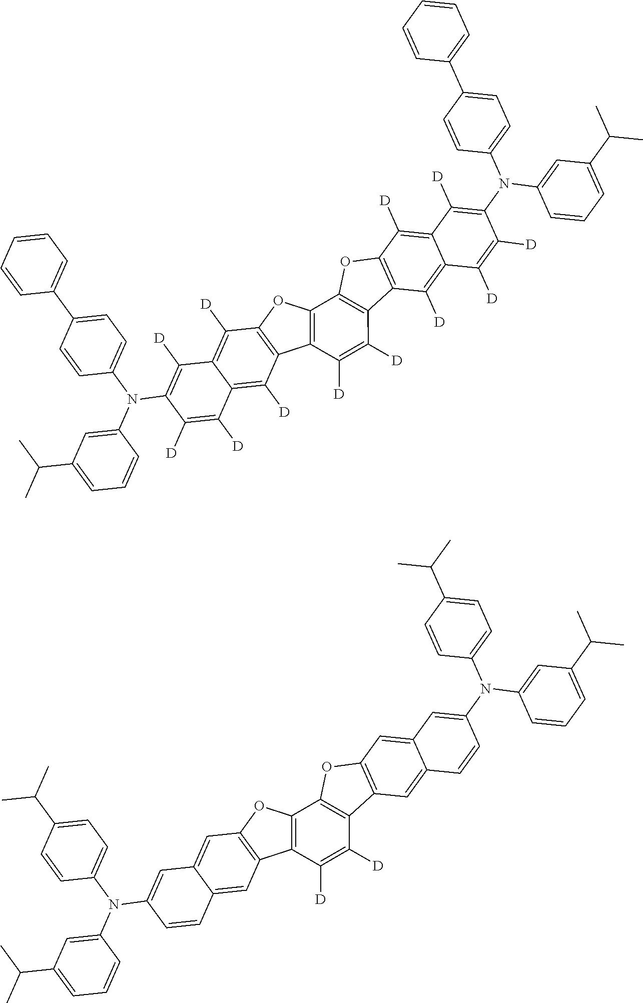

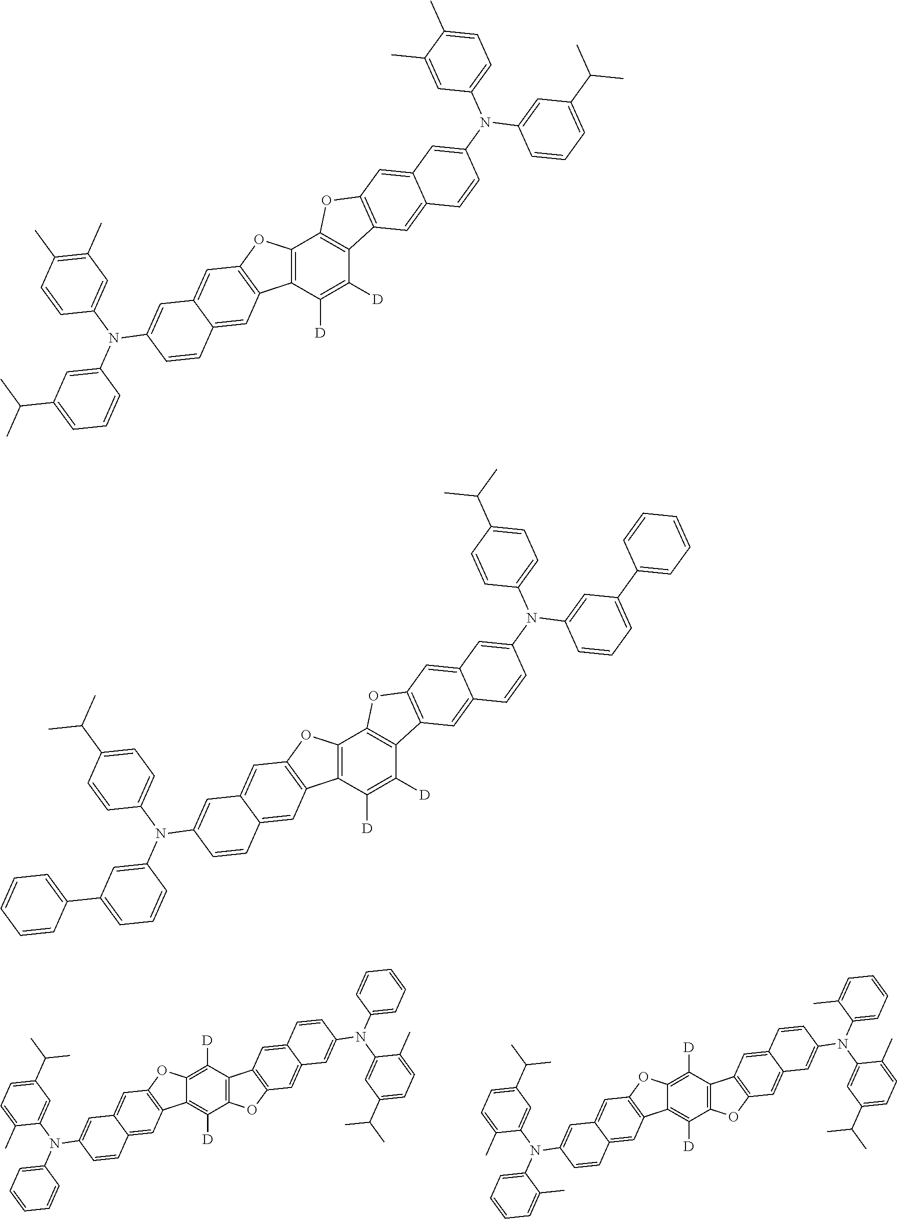 [SMILES: [2H]c1c([2H])c2c3cc4ccc(N(c5cccc(C(C)C)c5)c5ccc(C)c(C)c5)cc4cc3oc2c2oc3cc4cc(N(c5cccc(C(C)C)c5)c5ccc(C)c(C)c5)ccc4cc3c12 ; [2H]c1c([2H])c2c3cc4ccc(N(c5ccc(C(C)C)cc5)c5cccc(-c6ccccc6)c5)cc4cc3oc2c2oc3cc4cc(N(c5ccc(C(C)C)cc5)c5cccc(-c6ccccc6)c5)ccc4cc3c12 ; [2H]c1c2oc3cc4cc(N(c5ccccc5)c5cc(C(C)C)ccc5C)ccc4cc3c2c([2H])c2oc3cc4cc(N(c5ccccc5)c5cc(C(C)C)ccc5C)ccc4cc3c12 ; [2H]c1c2oc3cc4cc(N(c5ccccc5C)c5cc(C(C)C)ccc5C)ccc4cc3c2c([2H])c2oc3cc4cc(N(c5ccccc5C)c5cc(C(C)C)ccc5C)ccc4cc3c12]

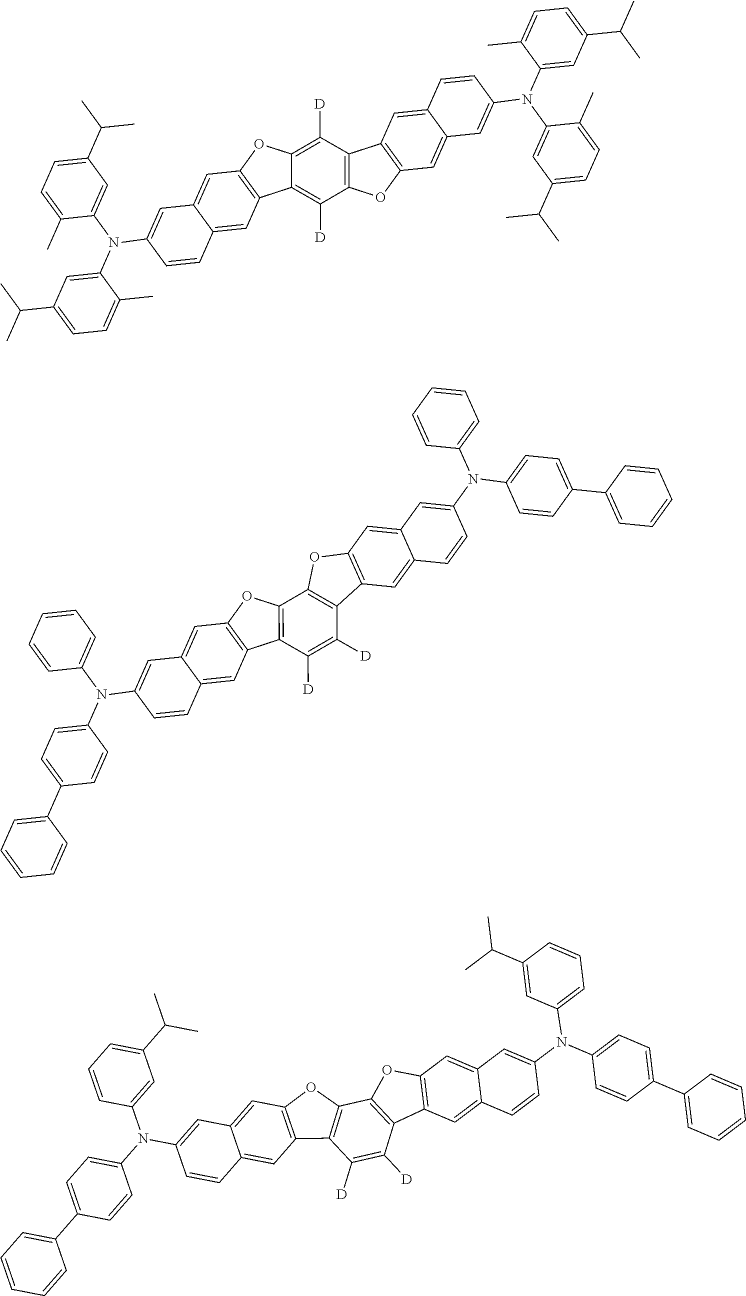 [SMILES: [2H]c1c2oc3cc4cc(N(c5cc(C(C)C)ccc5C)c5cc(C(C)C)ccc5C)ccc4cc3c2c([2H])c2oc3cc4cc(N(c5cc(C(C)C)ccc5C)c5cc(C(C)C)ccc5C)ccc4cc3c12 ; [2H]c1c([2H])c2c3cc4ccc(N(c5ccccc5)c5ccc(-c6ccccc6)cc5)cc4cc3oc2c2oc3cc4cc(N(c5ccccc5)c5ccc(-c6ccccc6)cc5)ccc4cc3c12 ; [2H]c1c([2H])c2c3cc4ccc(N(c5ccc(-c6ccccc6)cc5)c5cccc(C(C)C)c5)cc4cc3oc2c2oc3cc4cc(N(c5ccc(-c6ccccc6)cc5)c5cccc(C(C)C)c5)ccc4cc3c12]

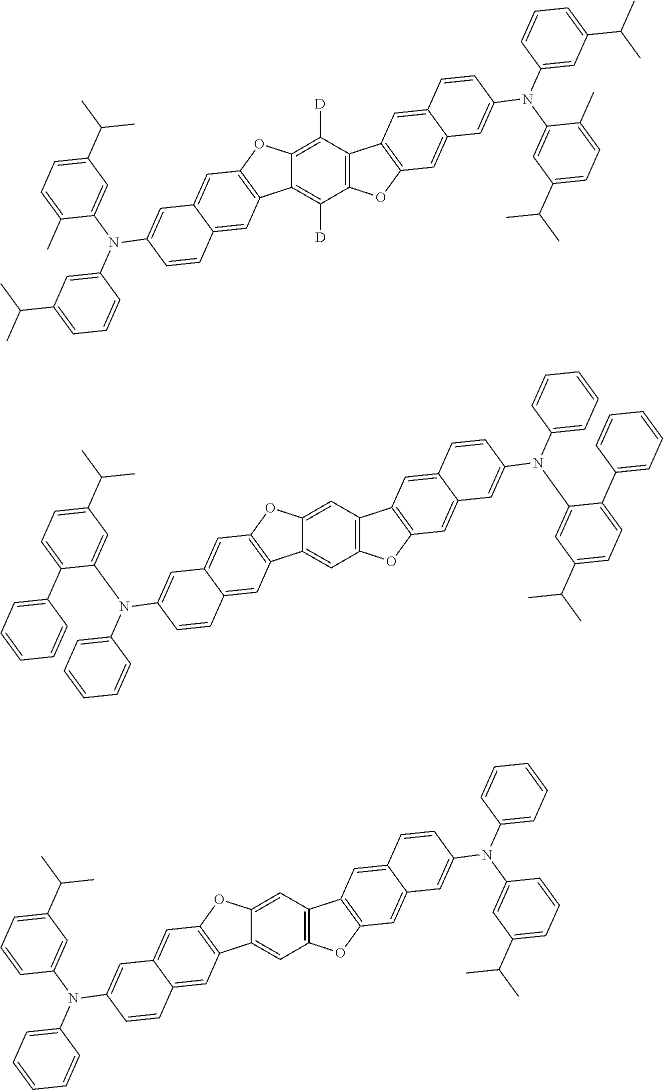 [SMILES: [2H]c1c2oc3cc4cc(N(c5cccc(C(C)C)c5)c5cc(C(C)C)ccc5C)ccc4cc3c2c([2H])c2oc3cc4cc(N(c5cccc(C(C)C)c5)c5cc(C(C)C)ccc5C)ccc4cc3c12 ; CC(C)c1ccc(-c2ccccc2)c(N(c2ccccc2)c2ccc3cc4c(cc3c2)oc2cc3c(cc24)oc2cc4cc(N(c5ccccc5)c5cc(C(C)C)ccc5-c5ccccc5)ccc4cc23)c1 ; CC(C)c1cccc(N(c2ccccc2)c2ccc3cc4c(cc3c2)oc2cc3c(cc24)oc2cc4cc(N(c5ccccc5)c5cccc(C(C)C)c5)ccc4cc23)c1]

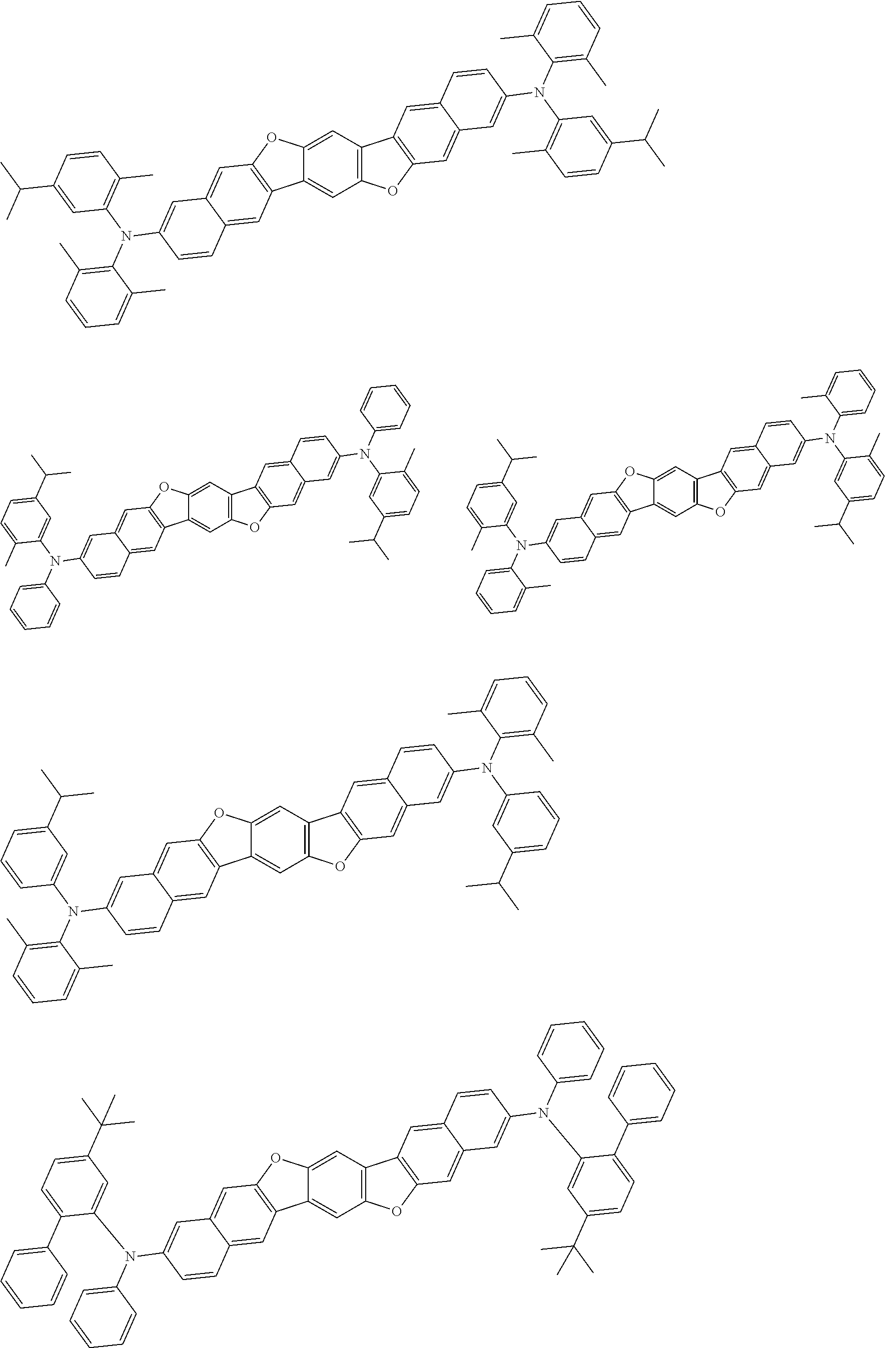

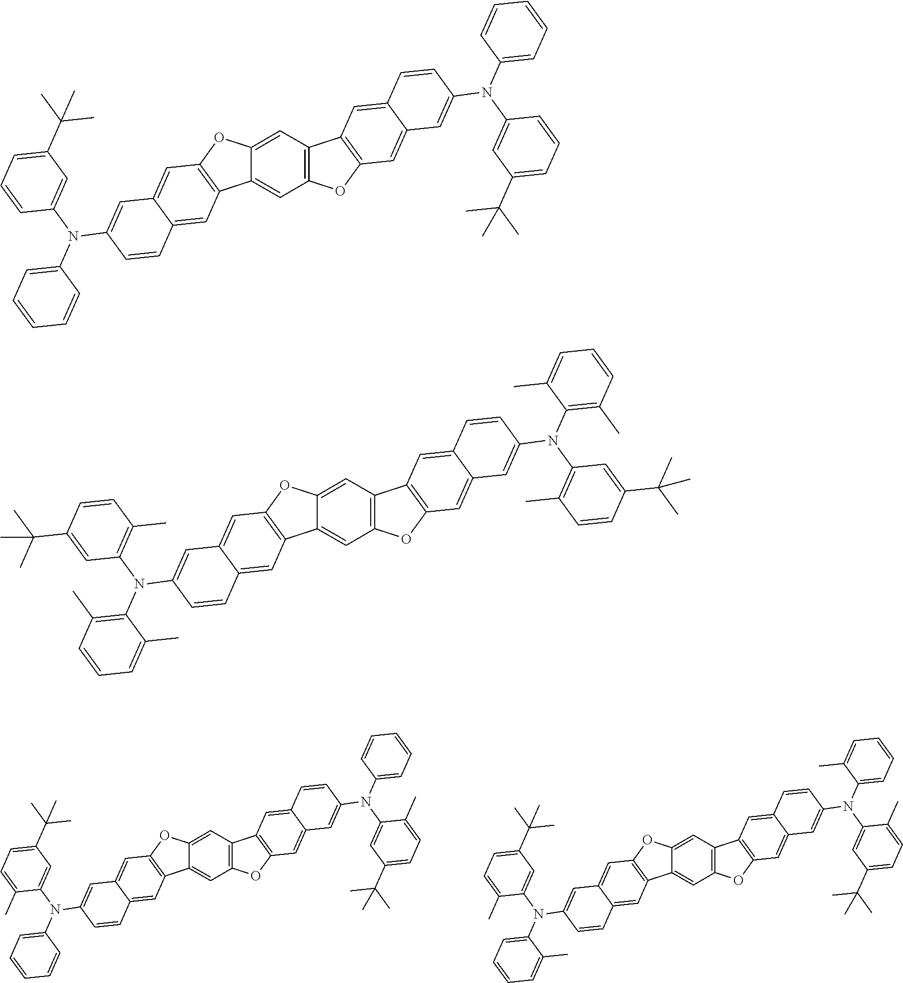 [SMILES: CC(C)(C)c1cccc(N(c2ccccc2)c2ccc3cc4c(cc3c2)oc2cc3c(cc24)oc2cc4cc(N(c5ccccc5)c5cccc(C(C)(C)C)c5)ccc4cc23)c1 ; Cc1ccc(C(C)(C)C)cc1N(c1ccc2cc3c(cc2c1)oc1cc2c(cc13)oc1cc3cc(N(c4cc(C(C)(C)C)ccc4C)c4c(C)cccc4C)ccc3cc12)c1c(C)cccc1C ; Cc1ccc(C(C)(C)C)cc1N(c1ccccc1)c1ccc2cc3c(cc2c1)oc1cc2c(cc13)oc1cc3cc(N(c4ccccc4)c4cc(C(C)(C)C)ccc4C)ccc3cc12 ; Cc1ccccc1N(c1ccc2cc3c(cc2c1)oc1cc2c(cc13)oc1cc3cc(N(c4ccccc4C)c4cc(C(C)(C)C)ccc4C)ccc3cc12)c1cc(C(C)(C)C)ccc1C]

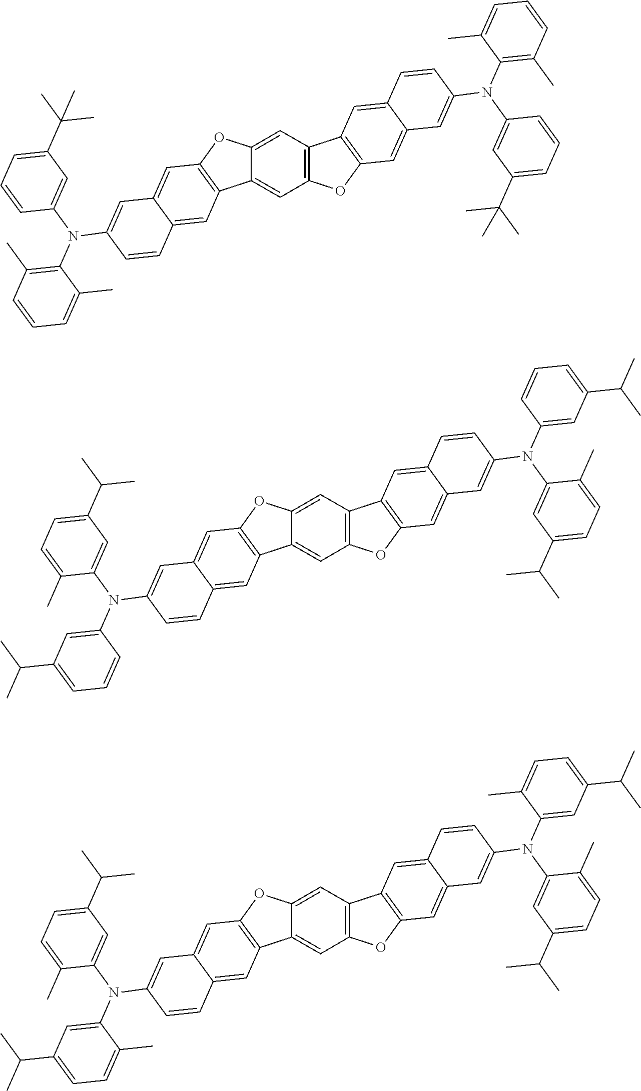

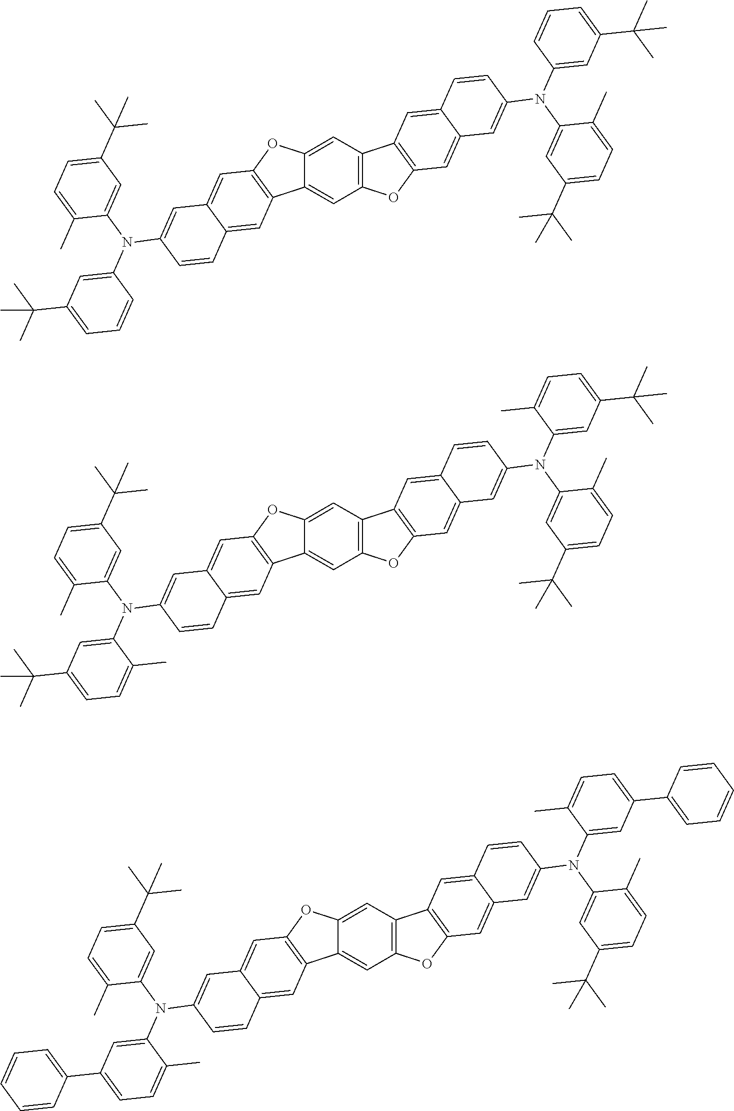

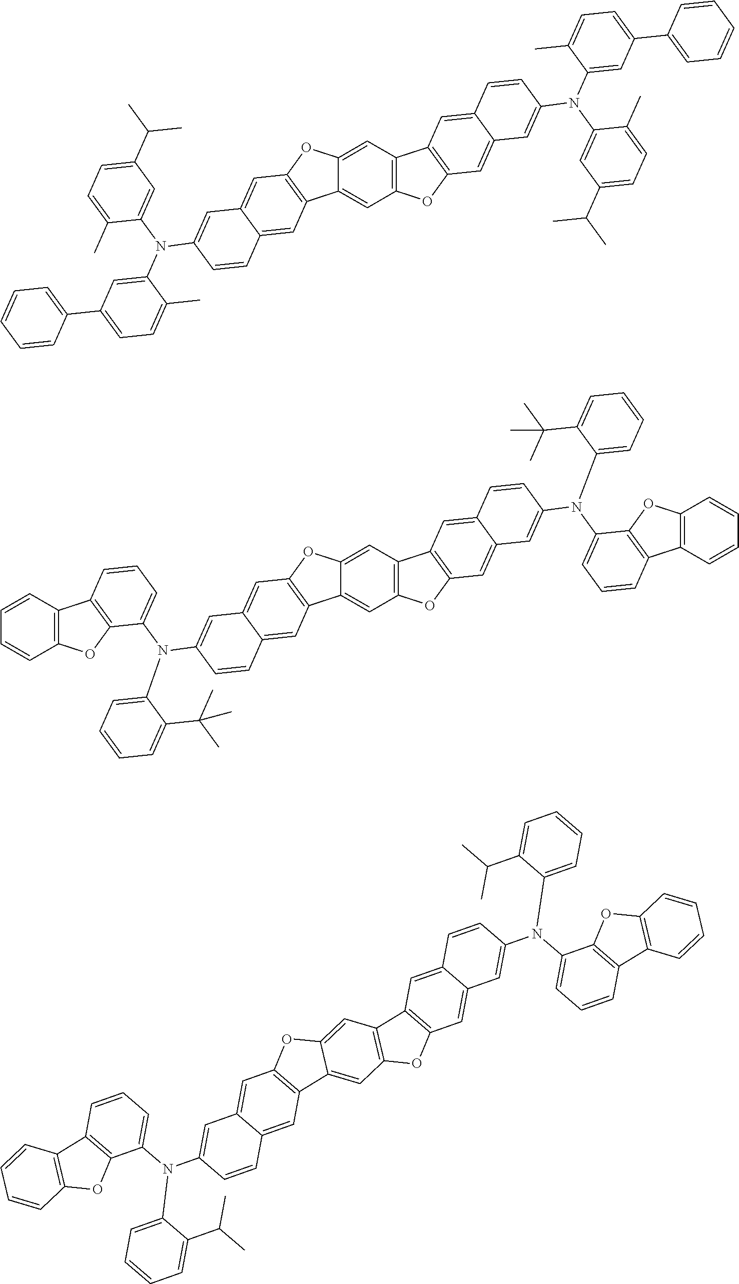 [SMILES: Cc1ccc(-c2ccccc2)cc1N(c1ccc2cc3c(cc2c1)oc1cc2c(cc13)oc1cc3cc(N(c4cc(-c5ccccc5)ccc4C)c4cc(C(C)C)ccc4C)ccc3cc12)c1cc(C(C)C)ccc1C ; CC(C)(C)c1ccccc1N(c1ccc2cc3c(cc2c1)oc1cc2c(cc13)oc1cc3cc(N(c4ccccc4C(C)(C)C)c4cccc5c4oc4ccccc45)ccc3cc12)c1cccc2c1oc1ccccc12 ; CC(C)c1ccccc1N(c1ccc2cc3c(cc2c1)oc1cc2c(cc13)oc1cc3cc(N(c4ccccc4C(C)C)c4cccc5c4oc4ccccc45)ccc3cc12)c1cccc2c1oc1ccccc12]

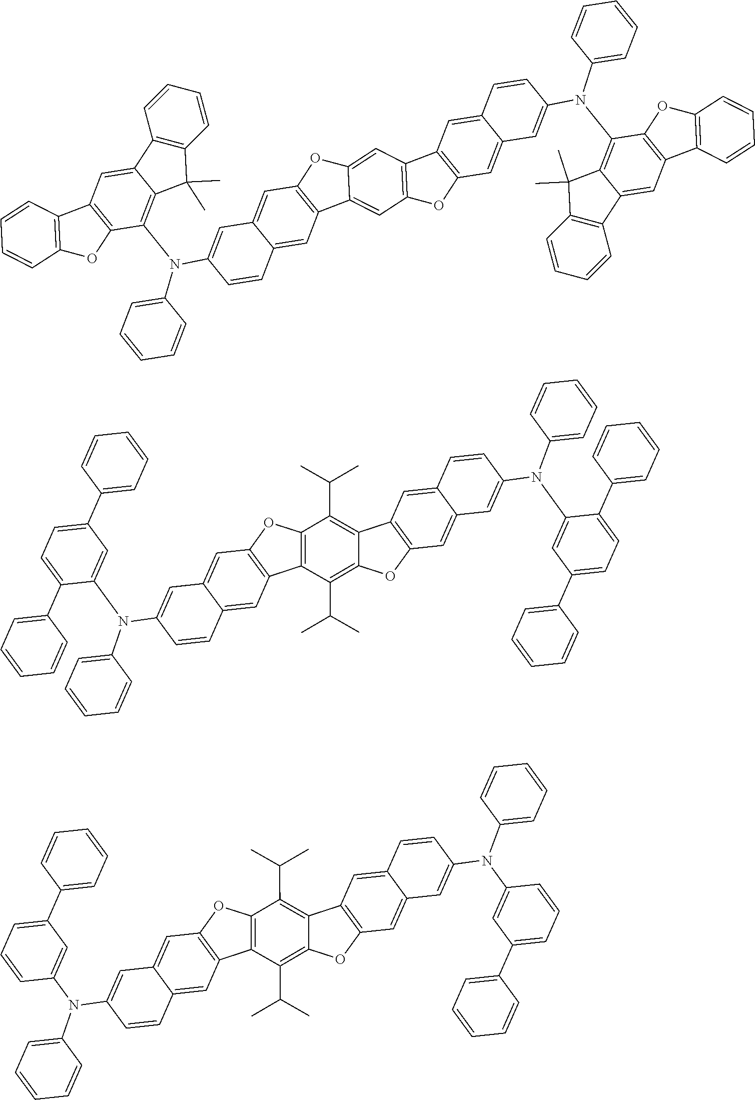 [SMILES: CC1(C)c2ccccc2-c2cc3c(oc4ccccc43)c(N(c3ccccc3)c3ccc4cc5c(cc4c3)oc3cc4c(cc35)oc3cc5cc(N(c6ccccc6)c6c7c(cc8c6oc6ccccc68)-c6ccccc6C7(C)C)ccc5cc34)c21 ; CC(C)c1c2oc3cc4cc(N(c5ccccc5)c5cc(-c6ccccc6)ccc5-c5ccccc5)ccc4cc3c2c(C(C)C)c2oc3cc4cc(N(c5ccccc5)c5cc(-c6ccccc6)ccc5-c5ccccc5)ccc4cc3c12 ; CC(C)c1c2oc3cc4cc(N(c5ccccc5)c5cccc(-c6ccccc6)c5)ccc4cc3c2c(C(C)C)c2oc3cc4cc(N(c5ccccc5)c5cccc(-c6ccccc6)c5)ccc4cc3c12]

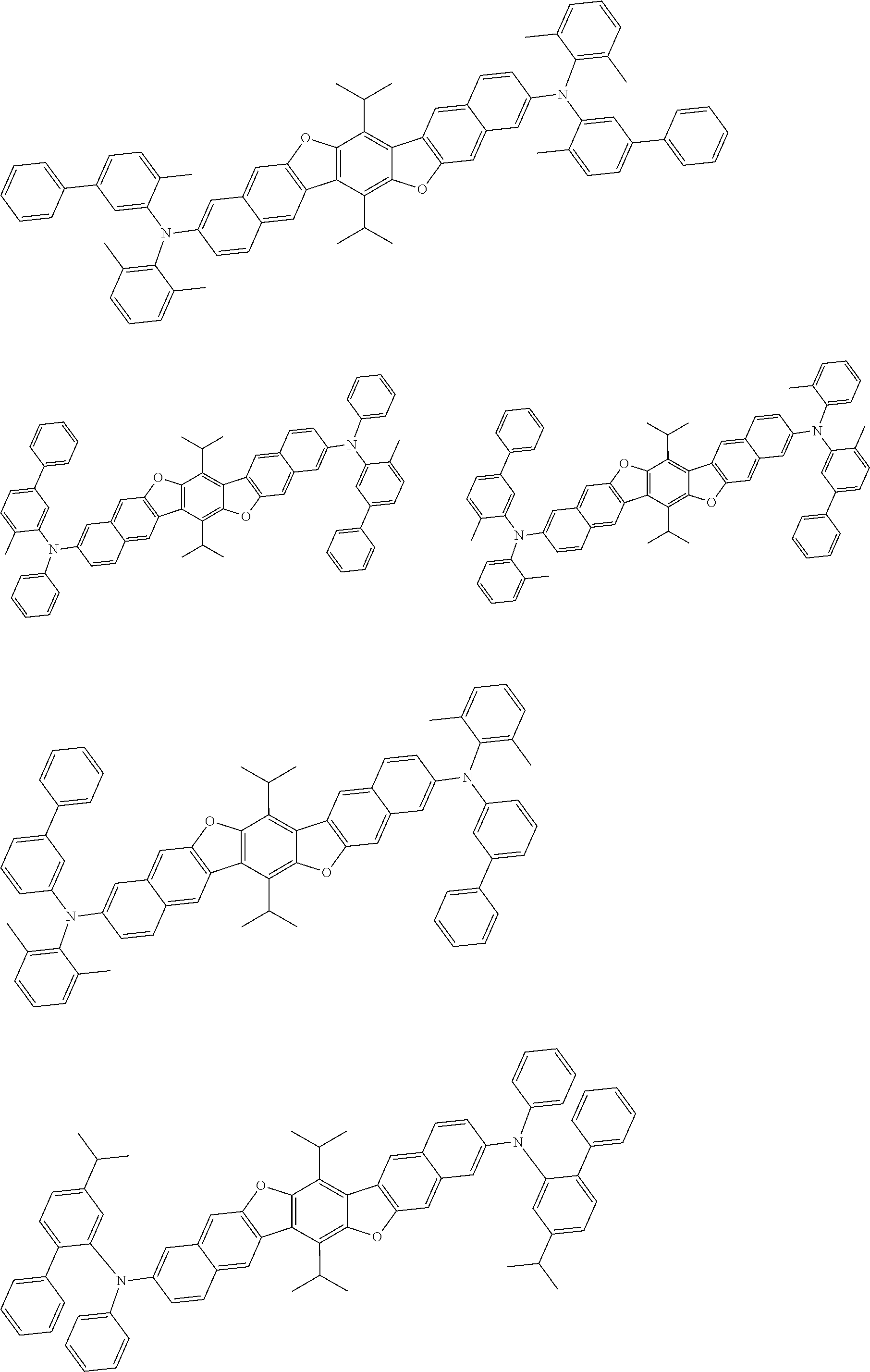

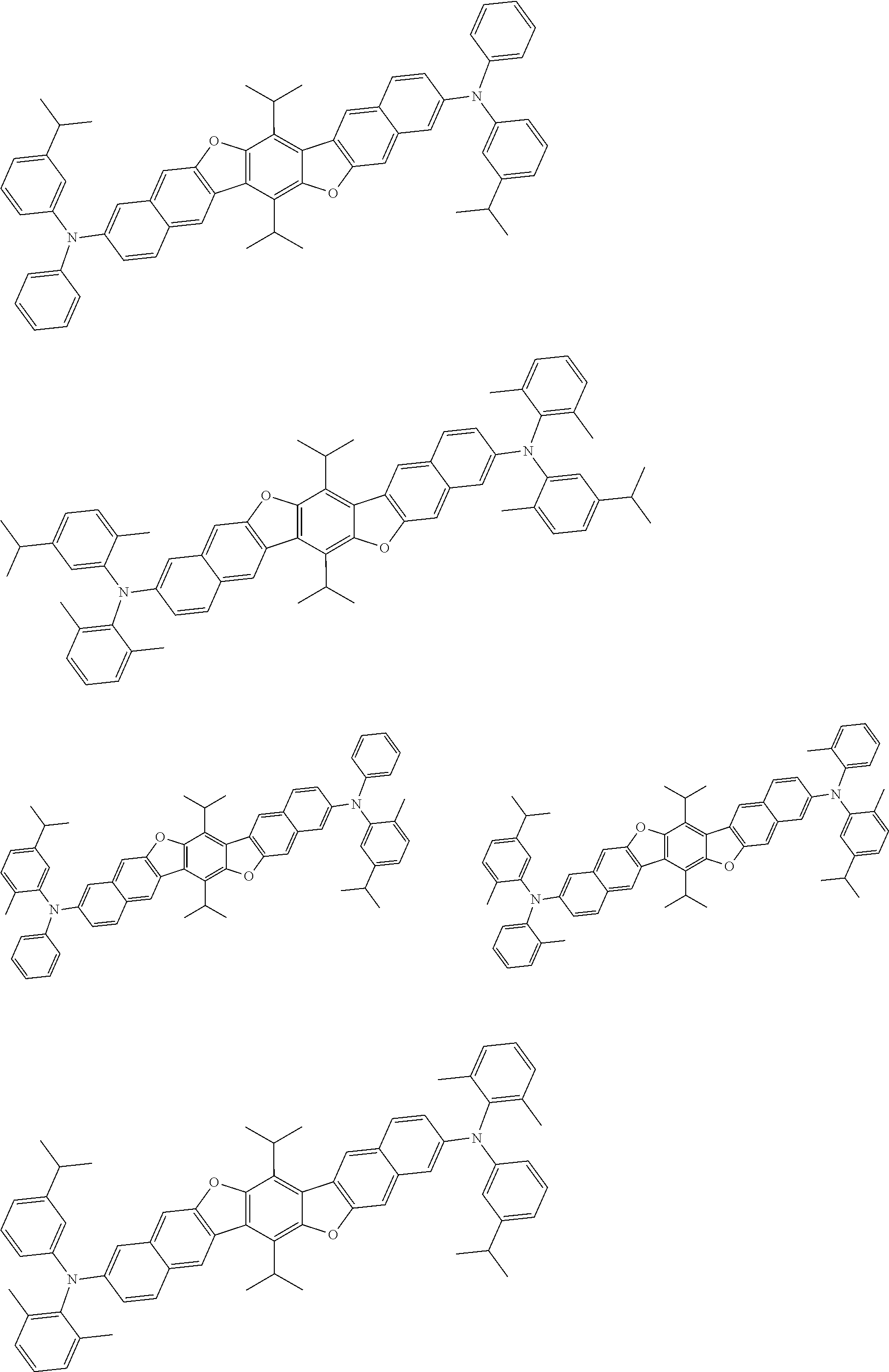

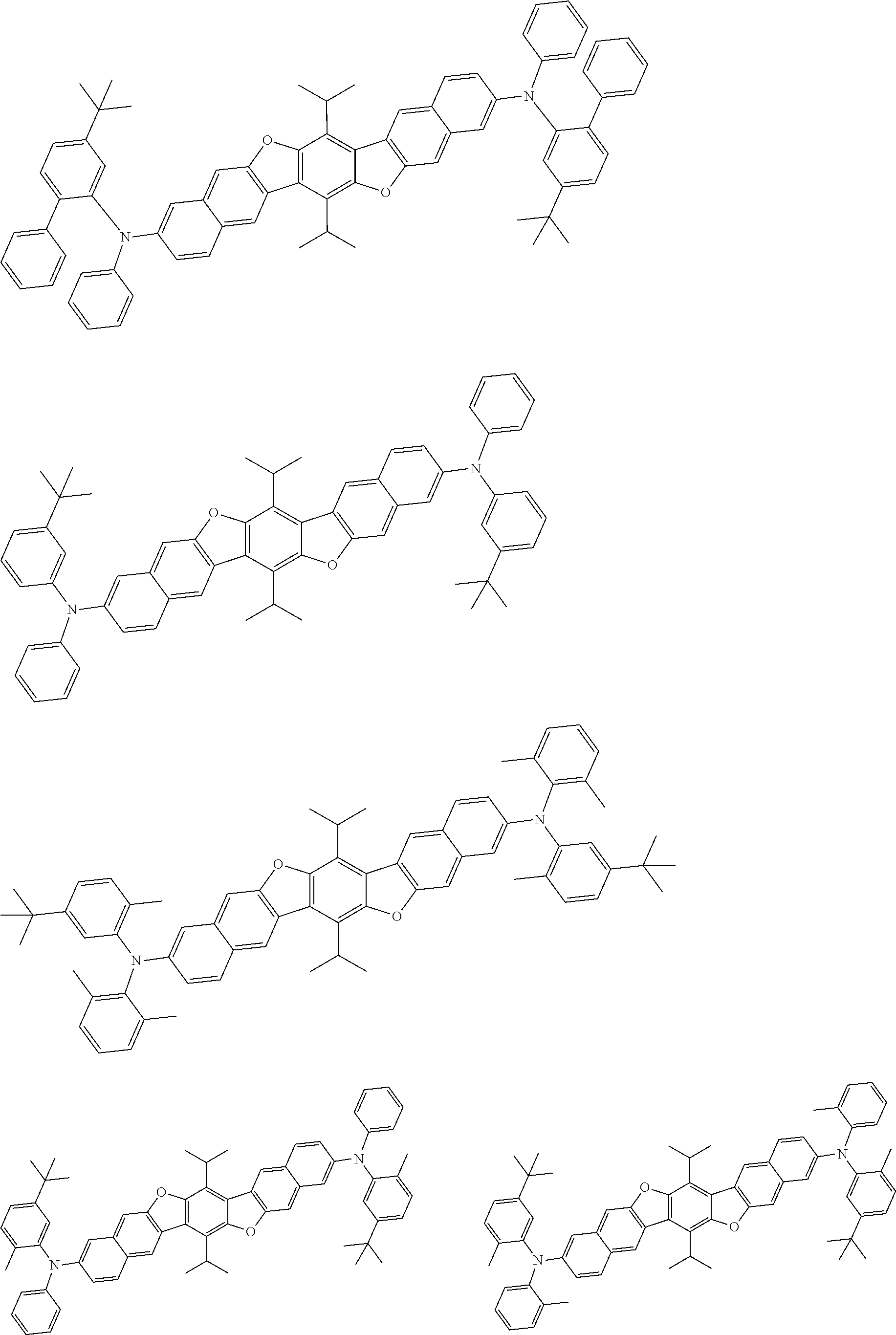

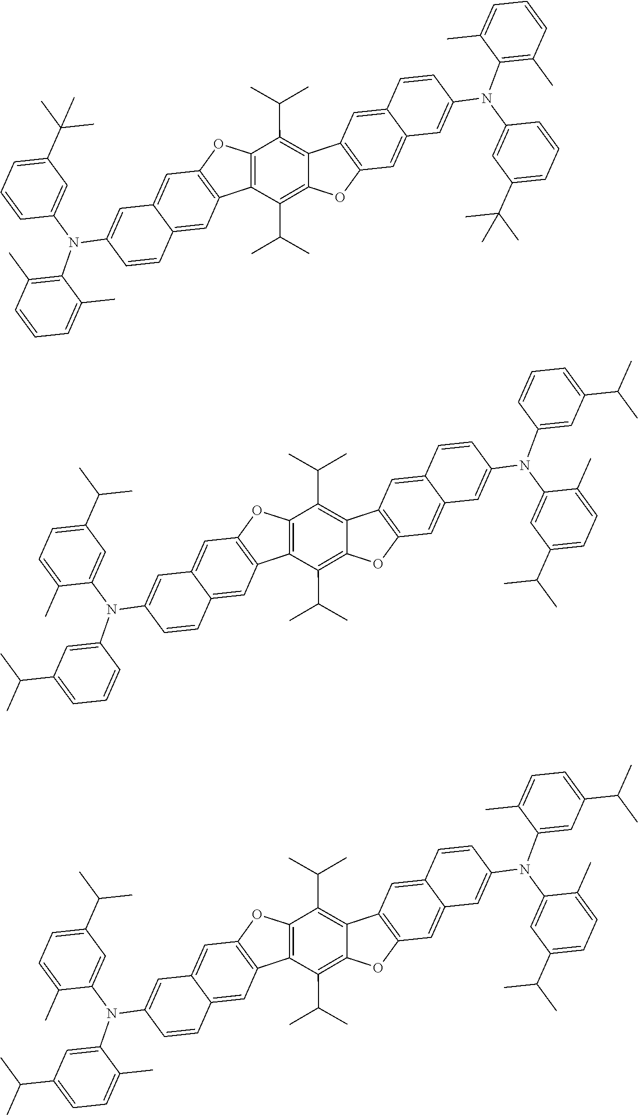

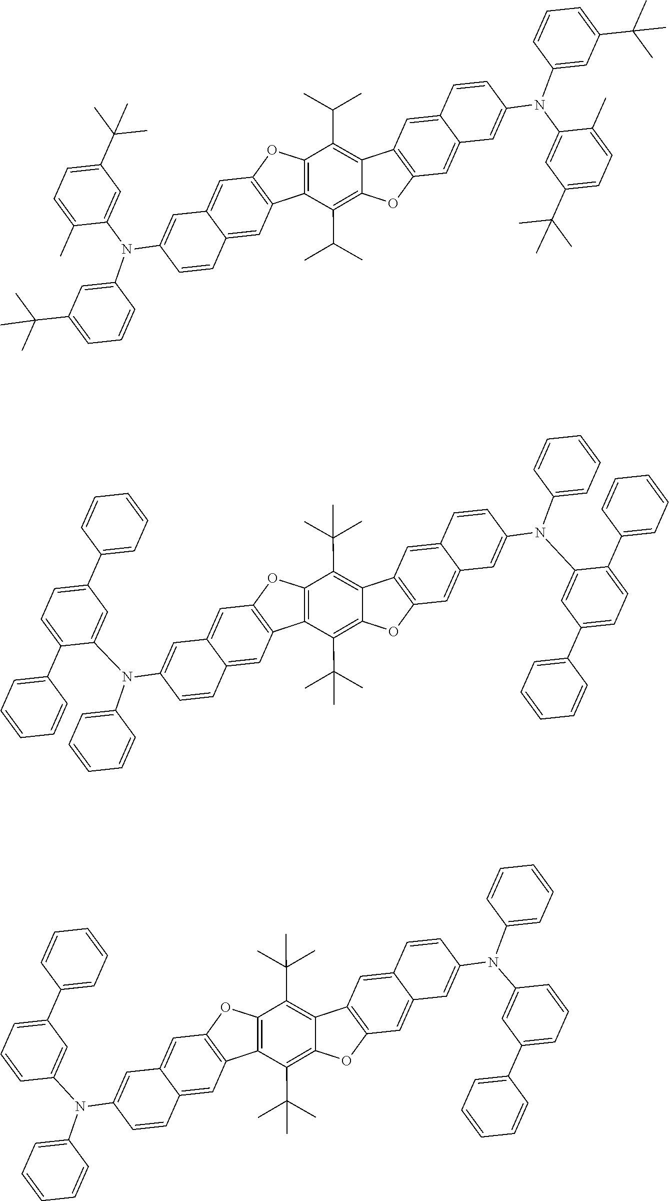 [SMILES: Cc1ccc(C(C)(C)C)cc1N(c1cccc(C(C)(C)C)c1)c1ccc2cc3c(cc2c1)oc1c(C(C)C)c2c(oc4cc5cc(N(c6cccc(C(C)(C)C)c6)c6cc(C(C)(C)C)ccc6C)ccc5cc42)c(C(C)C)c13 ; CC(C)(C)c1c2oc3cc4cc(N(c5ccccc5)c5cc(-c6ccccc6)ccc5-c5ccccc5)ccc4cc3c2c(C(C)(C)C)c2oc3cc4cc(N(c5ccccc5)c5cc(-c6ccccc6)ccc5-c5ccccc5)ccc4cc3c12 ; CC(C)(C)c1c2oc3cc4cc(N(c5ccccc5)c5cccc(-c6ccccc6)c5)ccc4cc3c2c(C(C)(C)C)c2oc3cc4cc(N(c5ccccc5)c5cccc(-c6ccccc6)c5)ccc4cc3c12]

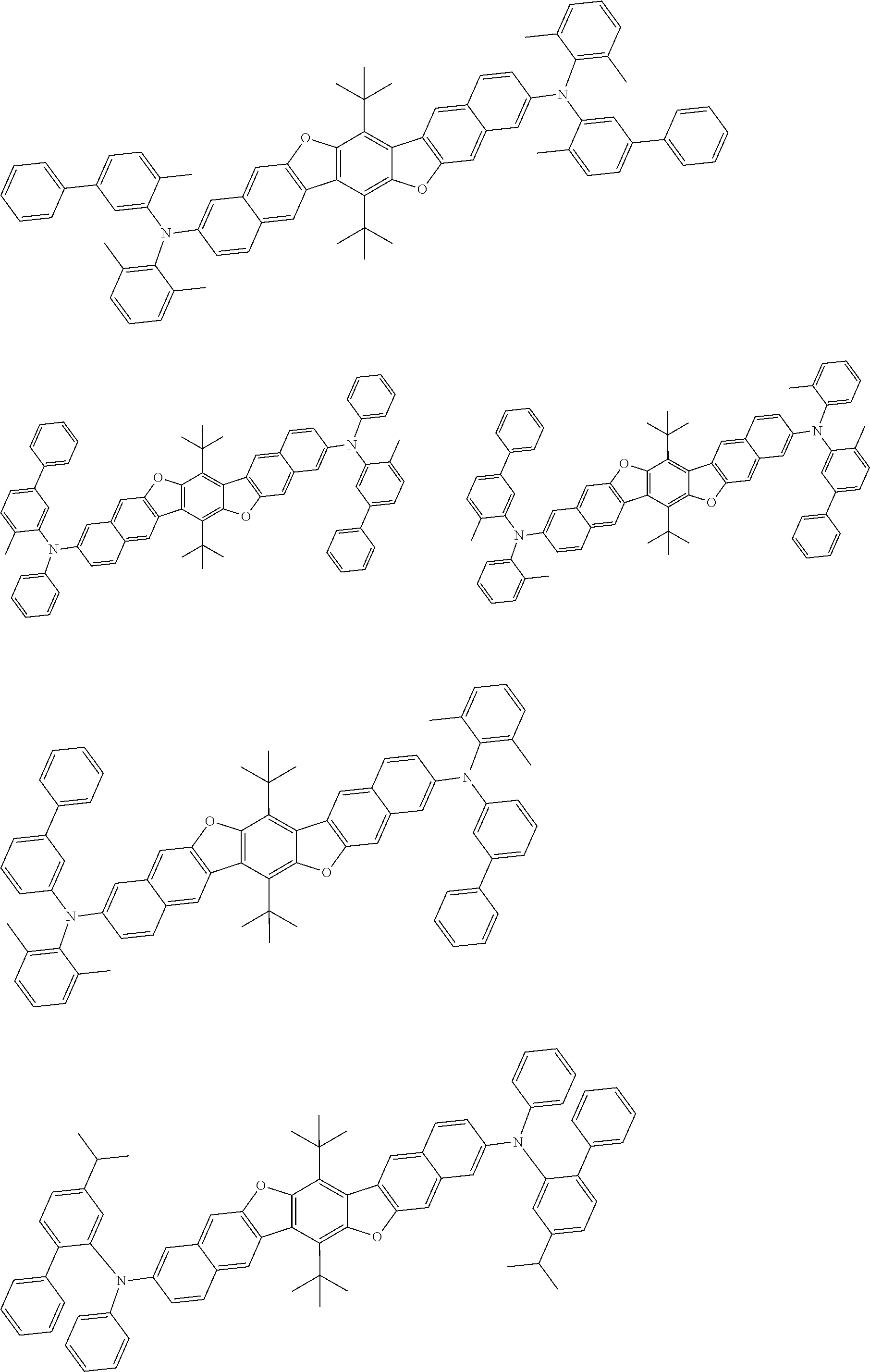

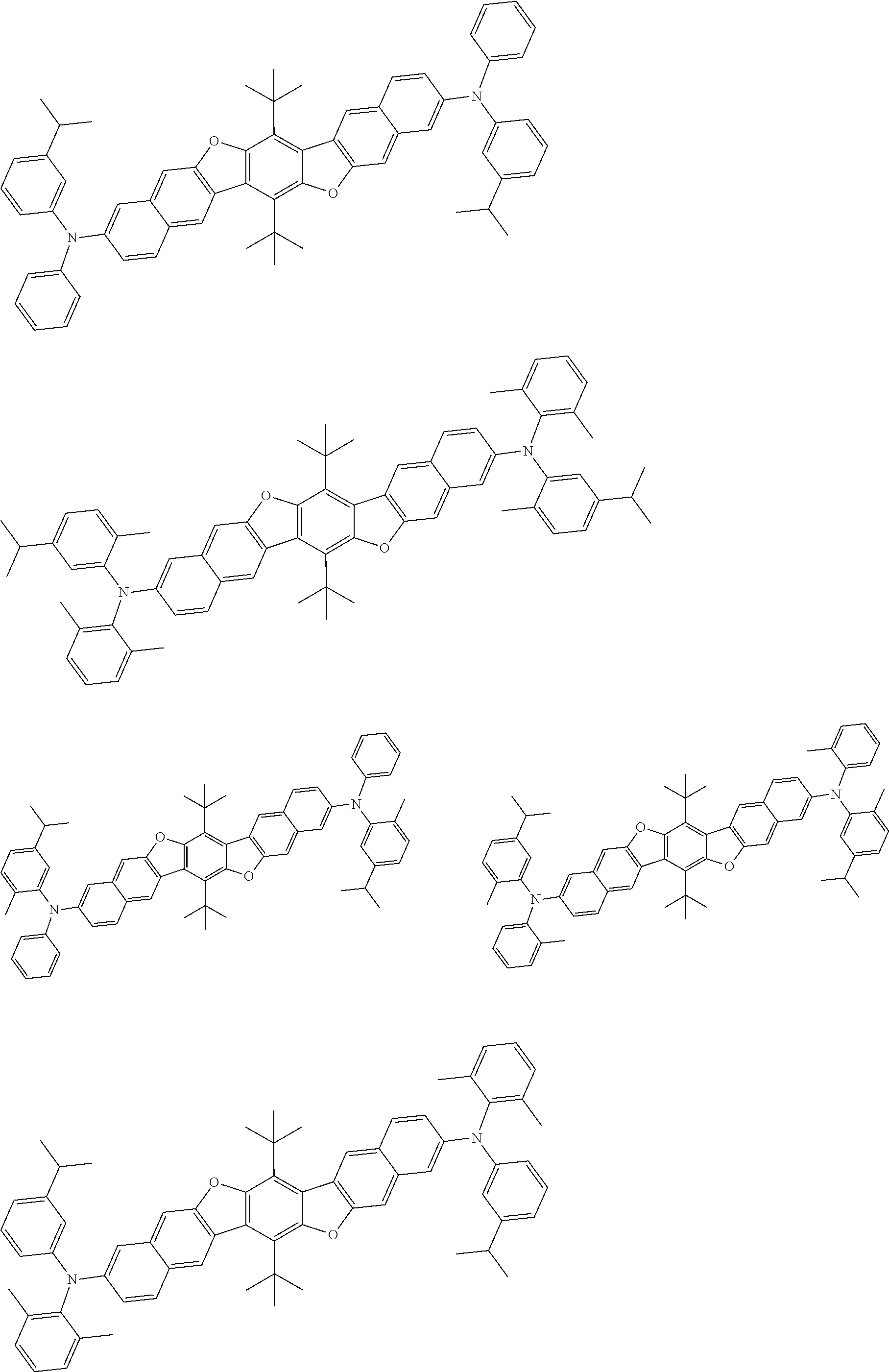

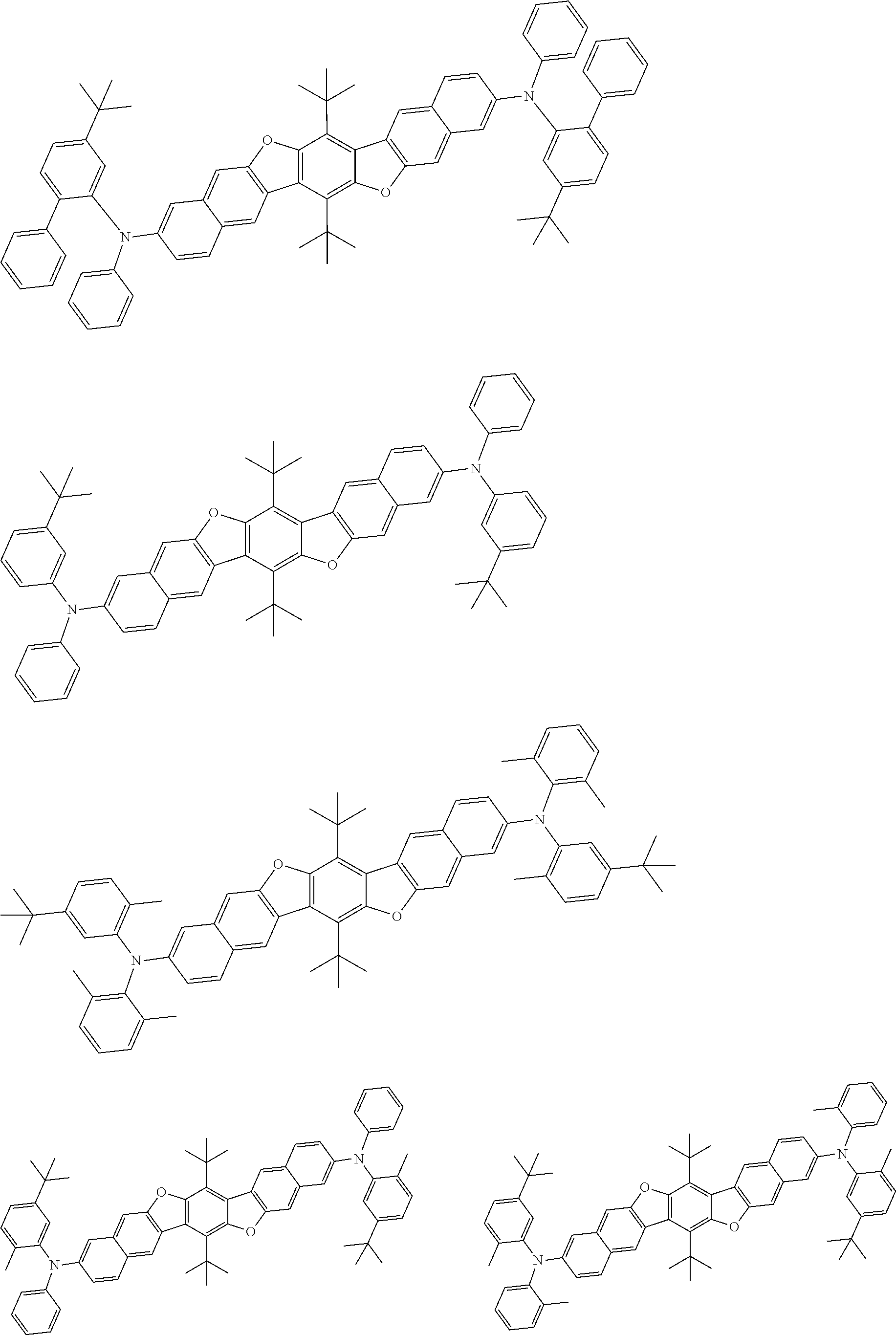

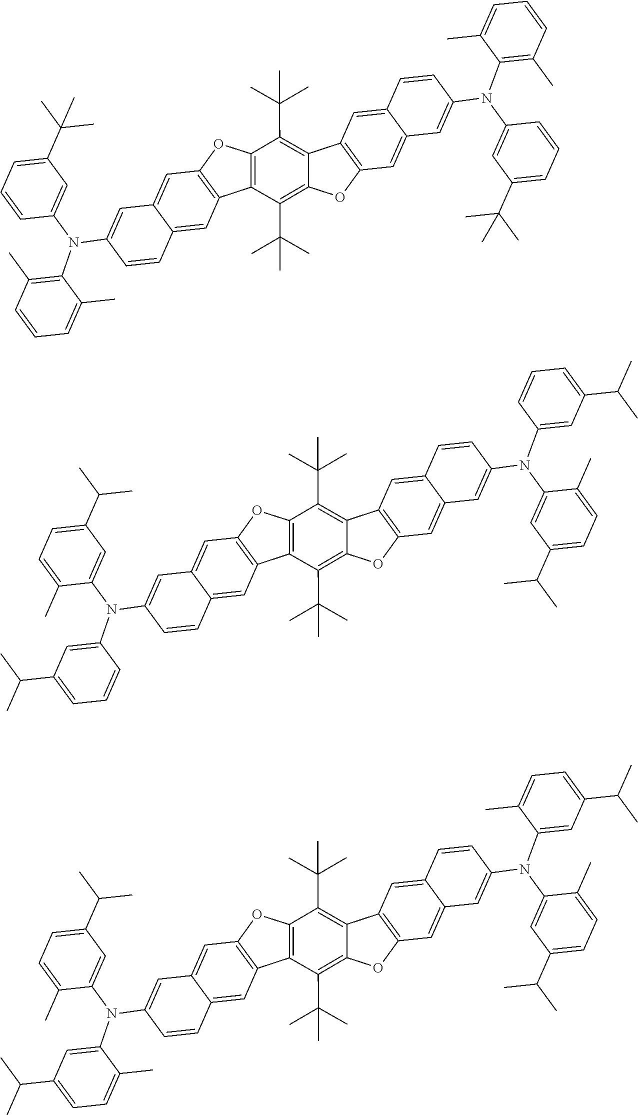

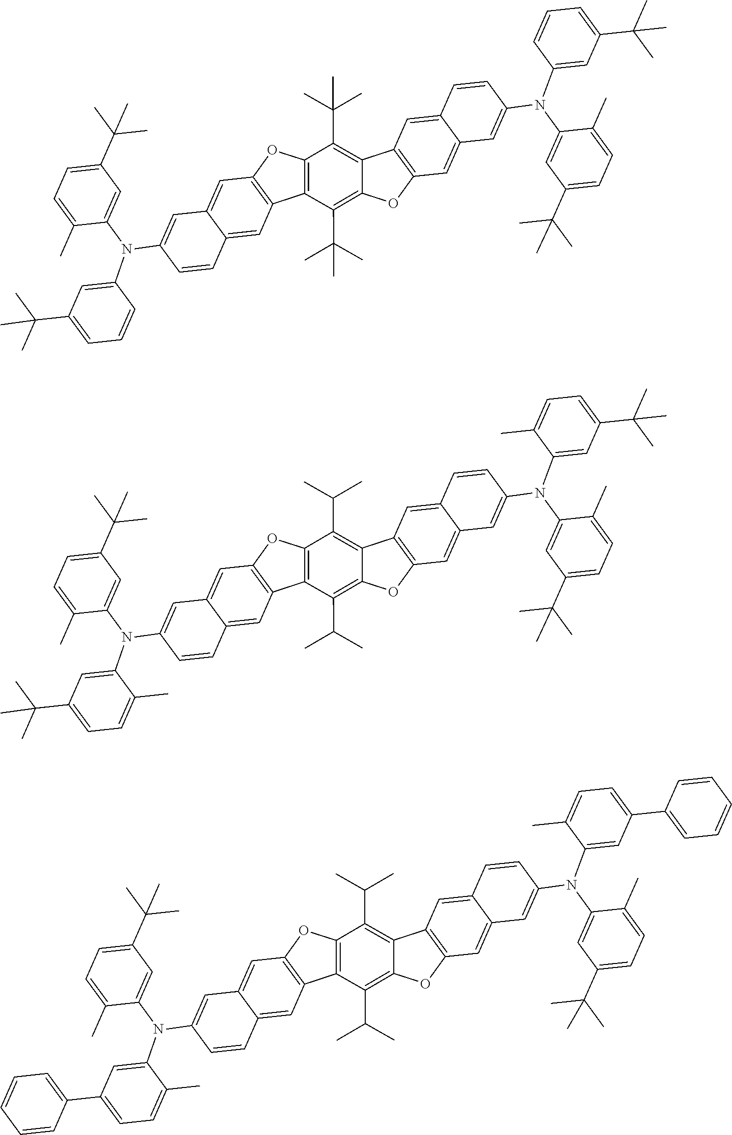

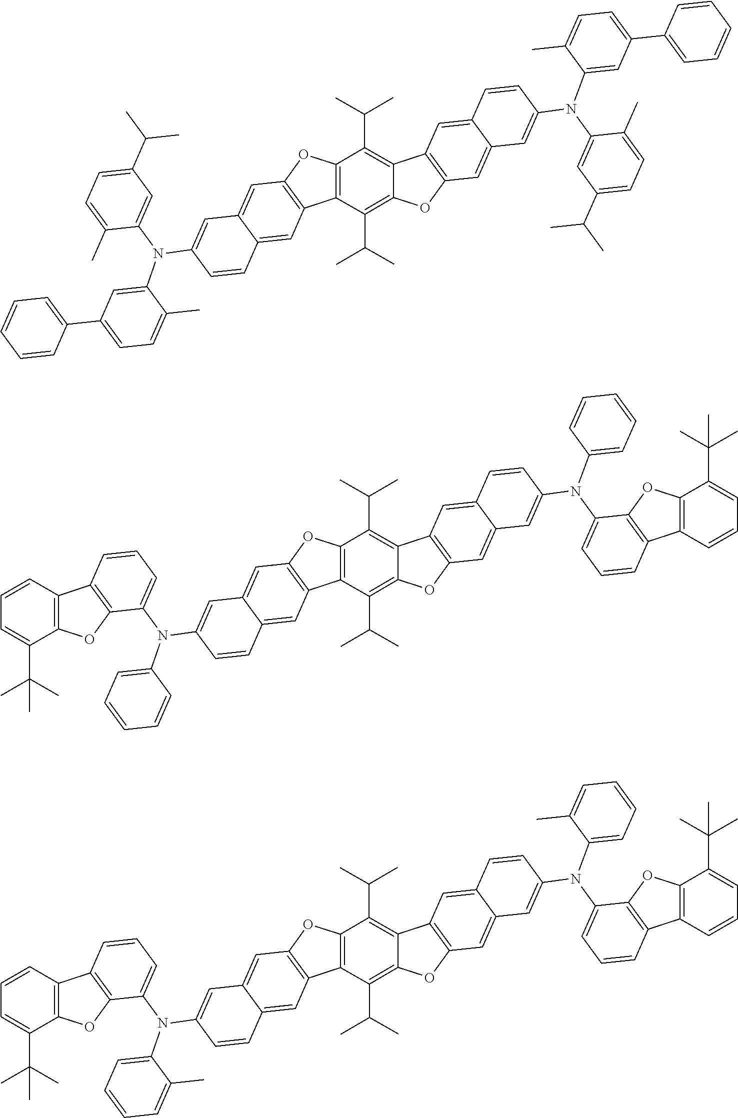 [SMILES: Cc1ccc(-c2ccccc2)cc1N(c1ccc2cc3c(cc2c1)oc1c(C(C)C)c2c(oc4cc5cc(N(c6cc(-c7ccccc7)ccc6C)c6cc(C(C)C)ccc6C)ccc5cc42)c(C(C)C)c13)c1cc(C(C)C)ccc1C ; CC(C)c1c2oc3cc4cc(N(c5ccccc5)c5cccc6c5oc5c(C(C)(C)C)cccc56)ccc4cc3c2c(C(C)C)c2oc3cc4cc(N(c5ccccc5)c5cccc6c5oc5c(C(C)(C)C)cccc56)ccc4cc3c12 ; Cc1ccccc1N(c1ccc2cc3c(cc2c1)oc1c(C(C)C)c2c(oc4cc5cc(N(c6ccccc6C)c6cccc7c6oc6c(C(C)(C)C)cccc67)ccc5cc42)c(C(C)C)c13)c1cccc2c1oc1c(C(C)(C)C)cccc12]

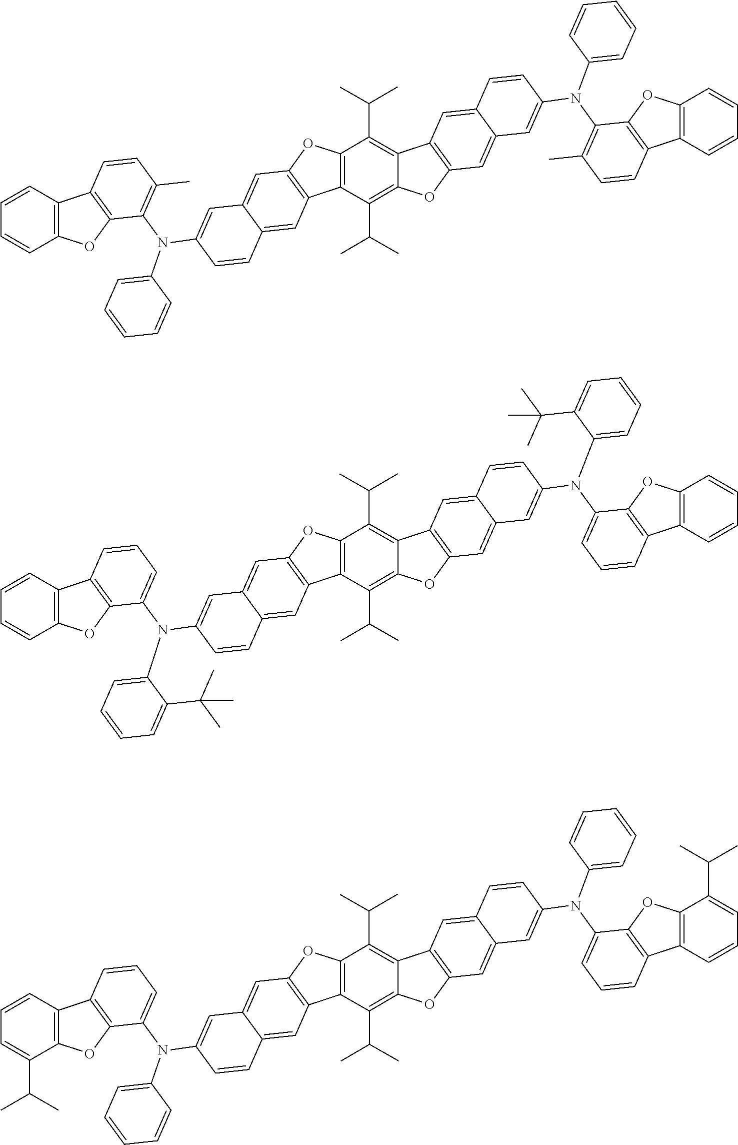 [SMILES: Cc1ccc2c(oc3ccccc32)c1N(c1ccccc1)c1ccc2cc3c(cc2c1)oc1c(C(C)C)c2c(oc4cc5cc(N(c6ccccc6)c6c(C)ccc7c6oc6ccccc67)ccc5cc42)c(C(C)C)c13 ; CC(C)c1c2oc3cc4cc(N(c5ccccc5C(C)(C)C)c5cccc6c5oc5ccccc56)ccc4cc3c2c(C(C)C)c2oc3cc4cc(N(c5ccccc5C(C)(C)C)c5cccc6c5oc5ccccc56)ccc4cc3c12 ; CC(C)c1cccc2c1oc1c(N(c3ccccc3)c3ccc4cc5c(cc4c3)oc3c(C(C)C)c4c(oc6cc7cc(N(c8ccccc8)c8cccc9c8oc8c(C(C)C)cccc89)ccc7cc64)c(C(C)C)c35)cccc12]

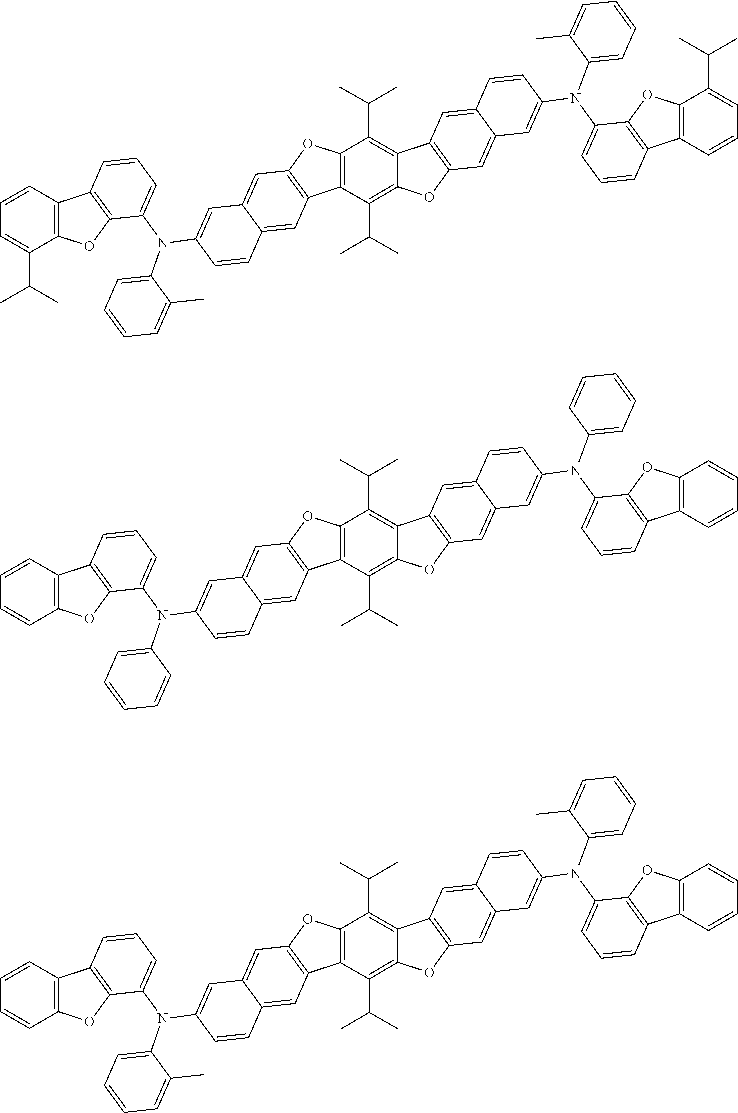 [SMILES: Cc1ccccc1N(c1ccc2cc3c(cc2c1)oc1c(C(C)C)c2c(oc4cc5cc(N(c6ccccc6C)c6cccc7c6oc6c(C(C)C)cccc67)ccc5cc42)c(C(C)C)c13)c1cccc2c1oc1c(C(C)C)cccc12 ; CC(C)c1c2oc3cc4cc(N(c5ccccc5)c5cccc6c5oc5ccccc56)ccc4cc3c2c(C(C)C)c2oc3cc4cc(N(c5ccccc5)c5cccc6c5oc5ccccc56)ccc4cc3c12 ; Cc1ccccc1N(c1ccc2cc3c(cc2c1)oc1c(C(C)C)c2c(oc4cc5cc(N(c6ccccc6C)c6cccc7c6oc6ccccc67)ccc5cc42)c(C(C)C)c13)c1cccc2c1oc1ccccc12]

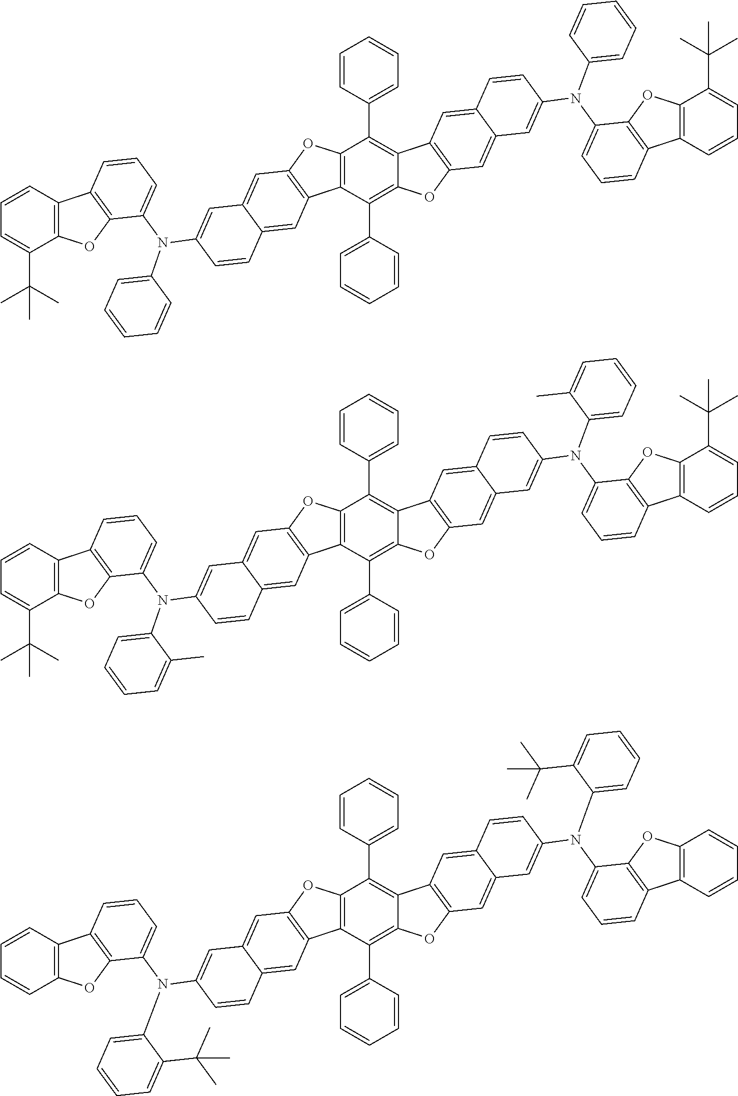 [SMILES: CC(C)(C)c1cccc2c1oc1c(N(c3ccccc3)c3ccc4cc5c(cc4c3)oc3c(-c4ccccc4)c4c(oc6cc7cc(N(c8ccccc8)c8cccc9c8oc8c(C(C)(C)C)cccc89)ccc7cc64)c(-c4ccccc4)c35)cccc12 ; Cc1ccccc1N(c1ccc2cc3c(cc2c1)oc1c(-c2ccccc2)c2c(oc4cc5cc(N(c6ccccc6C)c6cccc7c6oc6c(C(C)(C)C)cccc67)ccc5cc42)c(-c2ccccc2)c13)c1cccc2c1oc1c(C(C)(C)C)cccc12 ; CC(C)(C)c1ccccc1N(c1ccc2cc3c(cc2c1)oc1c(-c2ccccc2)c2c(oc4cc5cc(N(c6ccccc6C(C)(C)C)c6cccc7c6oc6ccccc67)ccc5cc42)c(-c2ccccc2)c13)c1cccc2c1oc1ccccc12]

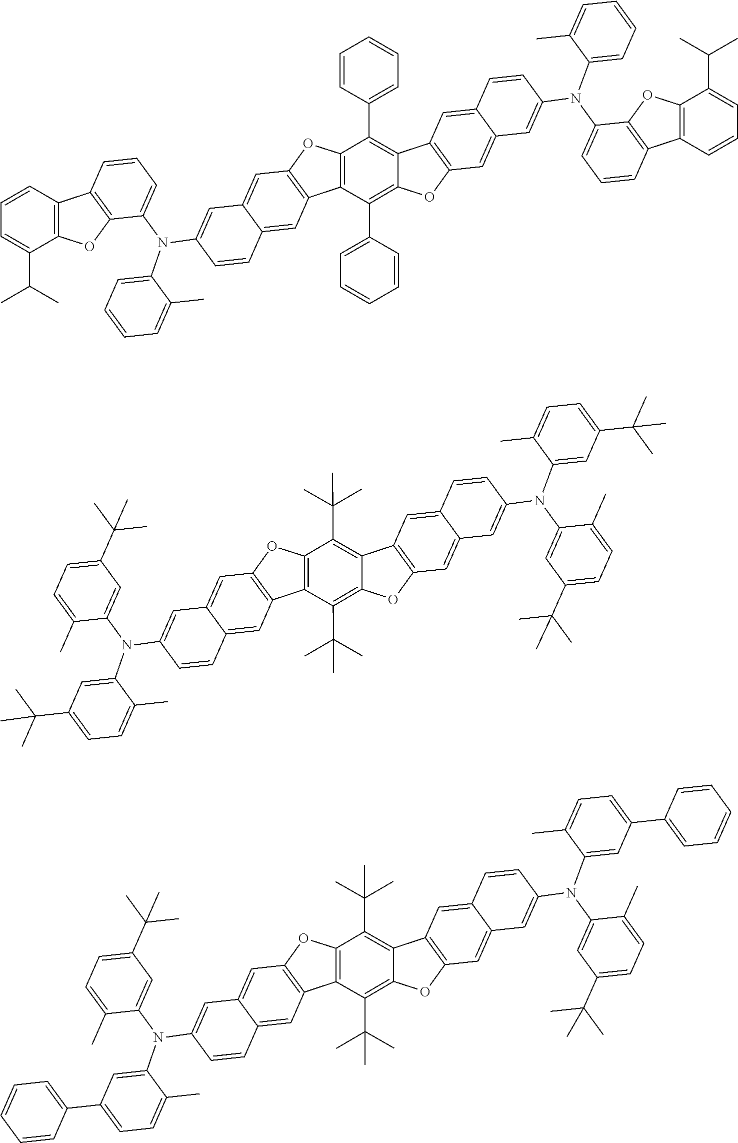

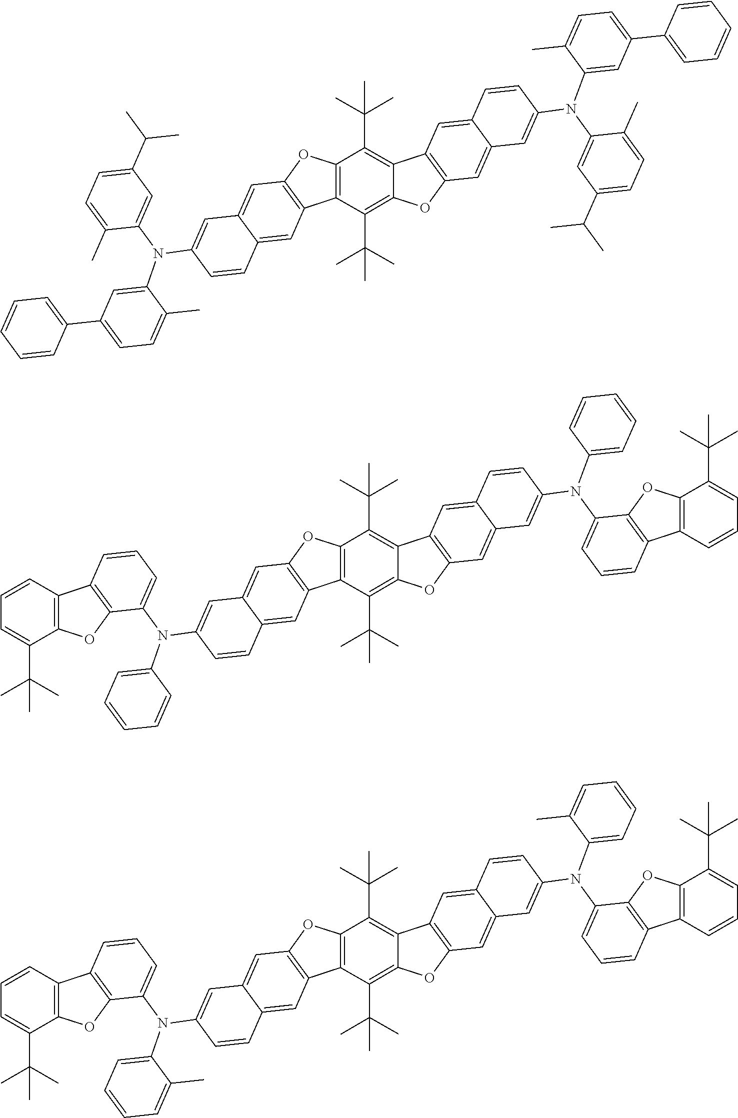 [SMILES: Cc1ccc(-c2ccccc2)cc1N(c1ccc2cc3c(cc2c1)oc1c(C(C)(C)C)c2c(oc4cc5cc(N(c6cc(-c7ccccc7)ccc6C)c6cc(C(C)C)ccc6C)ccc5cc42)c(C(C)(C)C)c13)c1cc(C(C)C)ccc1C ; CC(C)(C)c1cccc2c1oc1c(N(c3ccccc3)c3ccc4cc5c(cc4c3)oc3c(C(C)(C)C)c4c(oc6cc7cc(N(c8ccccc8)c8cccc9c8oc8c(C(C)(C)C)cccc89)ccc7cc64)c(C(C)(C)C)c35)cccc12 ; Cc1ccccc1N(c1ccc2cc3c(cc2c1)oc1c(C(C)(C)C)c2c(oc4cc5cc(N(c6ccccc6C)c6cccc7c6oc6c(C(C)(C)C)cccc67)ccc5cc42)c(C(C)(C)C)c13)c1cccc2c1oc1c(C(C)(C)C)cccc12]

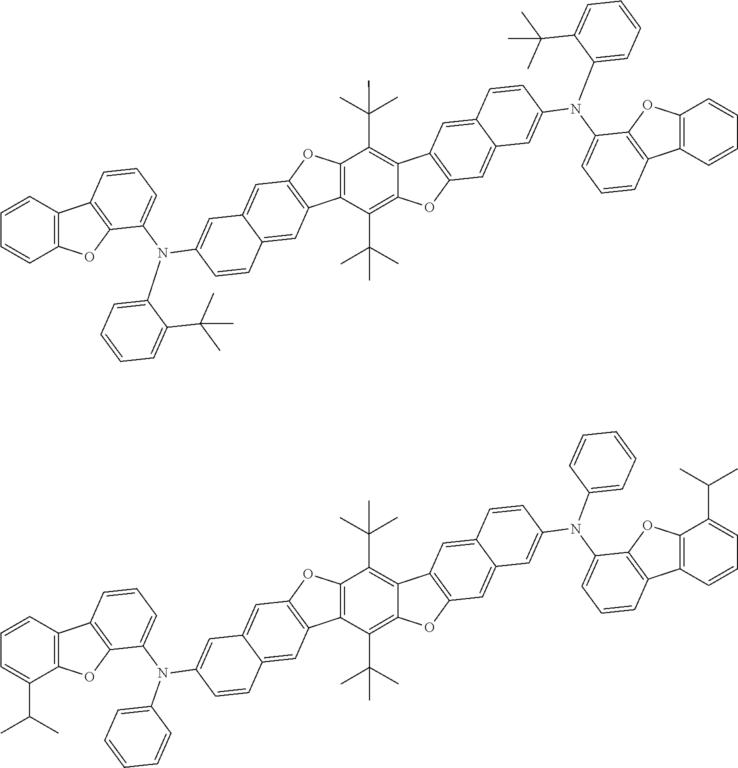 [SMILES: CC(C)(C)c1ccccc1N(c1ccc2cc3c(cc2c1)oc1c(C(C)(C)C)c2c(oc4cc5cc(N(c6ccccc6C(C)(C)C)c6cccc7c6oc6ccccc67)ccc5cc42)c(C(C)(C)C)c13)c1cccc2c1oc1ccccc12 ; CC(C)c1cccc2c1oc1c(N(c3ccccc3)c3ccc4cc5c(cc4c3)oc3c(C(C)(C)C)c4c(oc6cc7cc(N(c8ccccc8)c8cccc9c8oc8c(C(C)C)cccc89)ccc7cc64)c(C(C)(C)C)c35)cccc12]

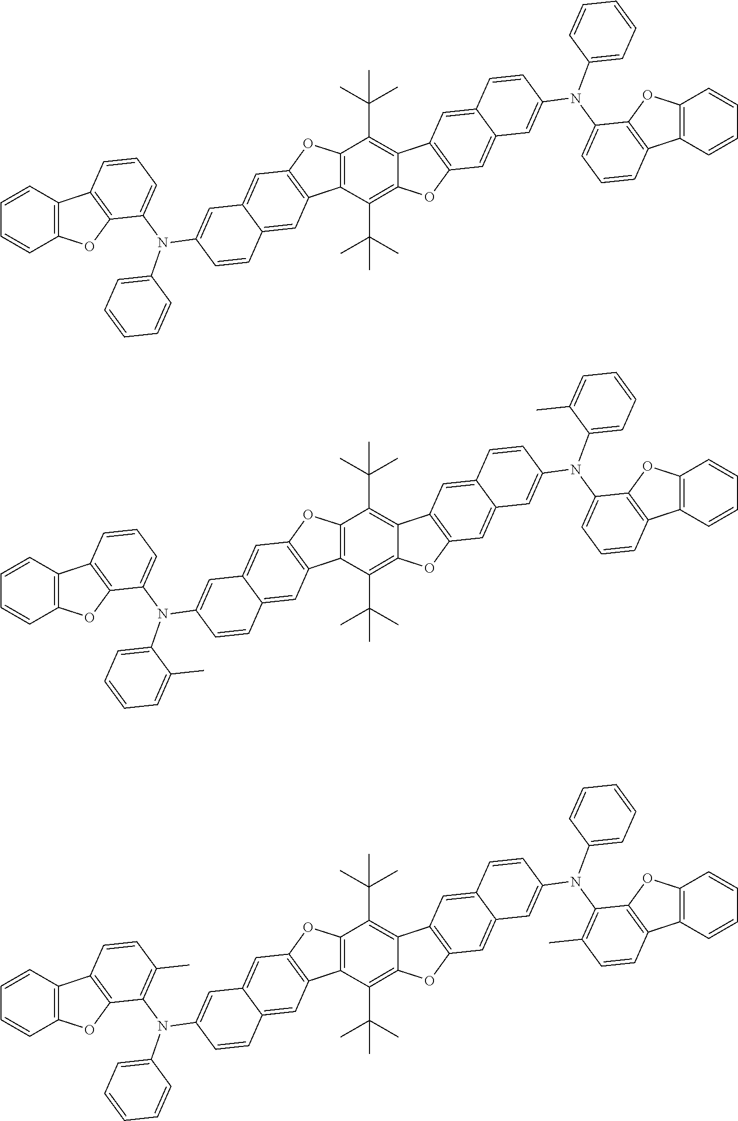 [SMILES: CC(C)(C)c1c2oc3cc4cc(N(c5ccccc5)c5cccc6c5oc5ccccc56)ccc4cc3c2c(C(C)(C)C)c2oc3cc4cc(N(c5ccccc5)c5cccc6c5oc5ccccc56)ccc4cc3c12 ; Cc1ccccc1N(c1ccc2cc3c(cc2c1)oc1c(C(C)(C)C)c2c(oc4cc5cc(N(c6ccccc6C)c6cccc7c6oc6ccccc67)ccc5cc42)c(C(C)(C)C)c13)c1cccc2c1oc1ccccc12 ; Cc1ccc2c(oc3ccccc32)c1N(c1ccccc1)c1ccc2cc3c(cc2c1)oc1c(C(C)(C)C)c2c(oc4cc5cc(N(c6ccccc6)c6c(C)ccc7c6oc6ccccc67)ccc5cc42)c(C(C)(C)C)c13]

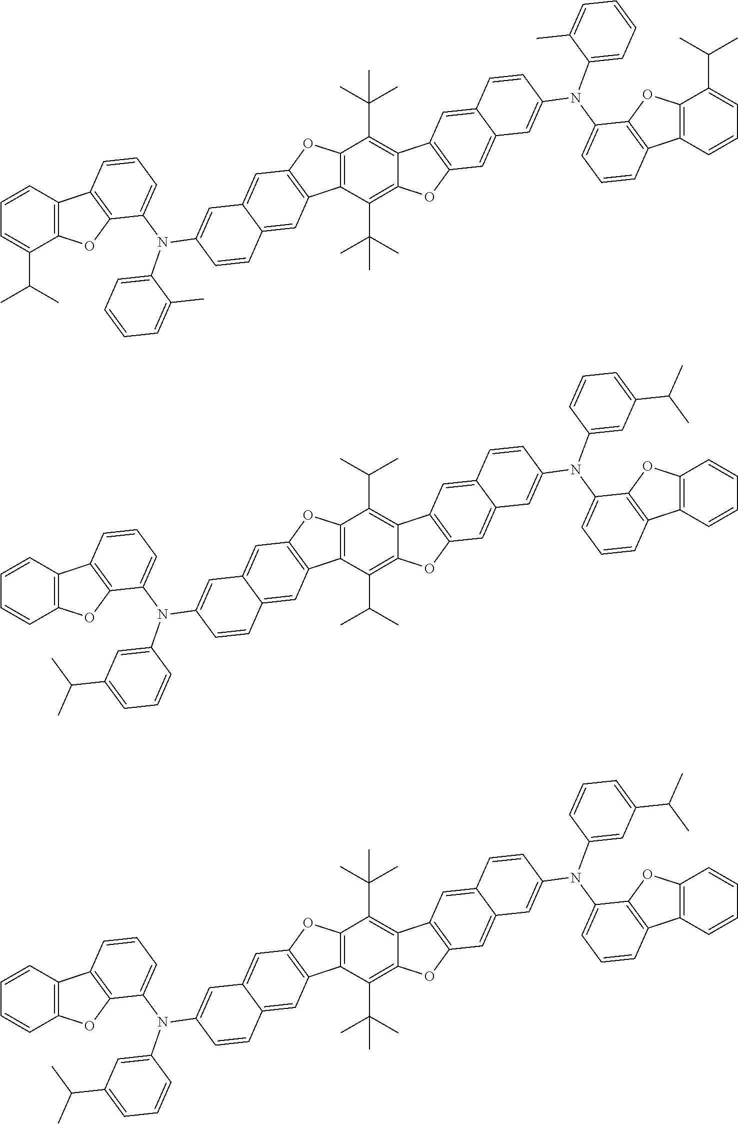 [SMILES: Cc1ccccc1N(c1ccc2cc3c(cc2c1)oc1c(C(C)(C)C)c2c(oc4cc5cc(N(c6ccccc6C)c6cccc7c6oc6c(C(C)C)cccc67)ccc5cc42)c(C(C)(C)C)c13)c1cccc2c1oc1c(C(C)C)cccc12 ; CC(C)c1cccc(N(c2ccc3cc4c(cc3c2)oc2c(C(C)C)c3c(oc5cc6cc(N(c7cccc(C(C)C)c7)c7cccc8c7oc7ccccc78)ccc6cc53)c(C(C)C)c24)c2cccc3c2oc2ccccc23)c1 ; CC(C)c1cccc(N(c2ccc3cc4c(cc3c2)oc2c(C(C)(C)C)c3c(oc5cc6cc(N(c7cccc(C(C)C)c7)c7cccc8c7oc7ccccc78)ccc6cc53)c(C(C)(C)C)c24)c2cccc3c2oc2ccccc23)c1]

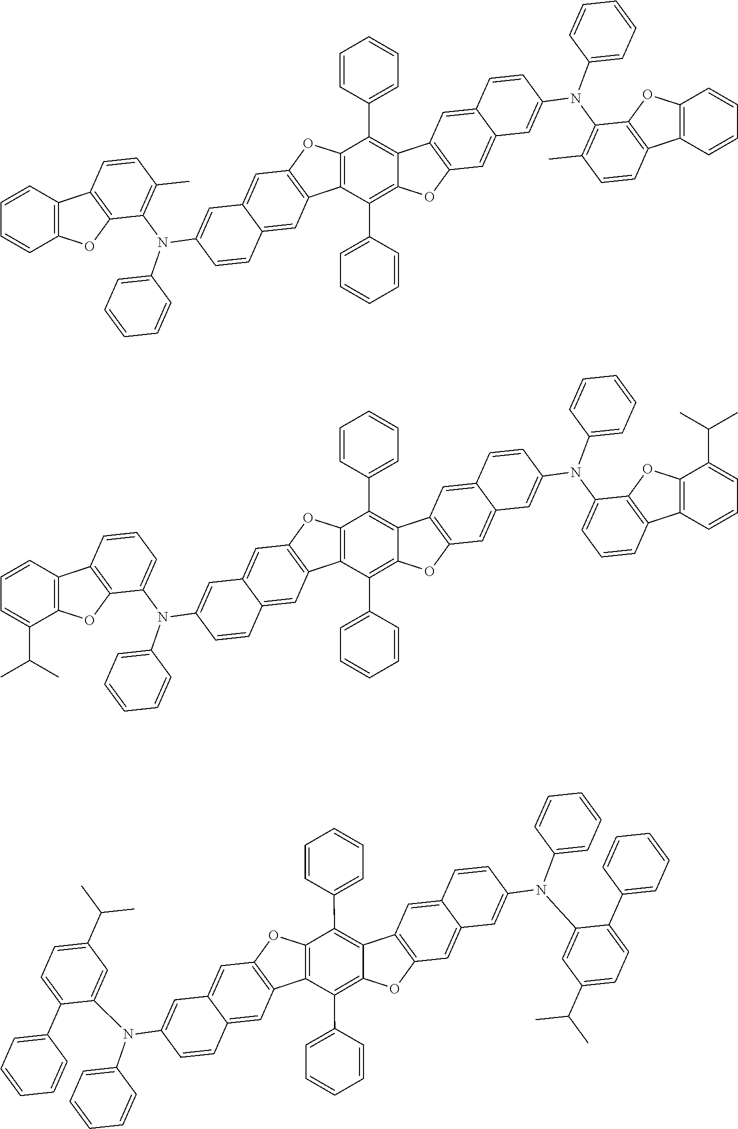 [SMILES: Cc1ccc2c(oc3ccccc32)c1N(c1ccccc1)c1ccc2cc3c(cc2c1)oc1c(-c2ccccc2)c2c(oc4cc5cc(N(c6ccccc6)c6c(C)ccc7c6oc6ccccc67)ccc5cc42)c(-c2ccccc2)c13 ; CC(C)c1cccc2c1oc1c(N(c3ccccc3)c3ccc4cc5c(cc4c3)oc3c(-c4ccccc4)c4c(oc6cc7cc(N(c8ccccc8)c8cccc9c8oc8c(C(C)C)cccc89)ccc7cc64)c(-c4ccccc4)c35)cccc12 ; CC(C)c1ccc(-c2ccccc2)c(N(c2ccccc2)c2ccc3cc4c(cc3c2)oc2c(-c3ccccc3)c3c(oc5cc6cc(N(c7ccccc7)c7cc(C(C)C)ccc7-c7ccccc7)ccc6cc53)c(-c3ccccc3)c24)c1]

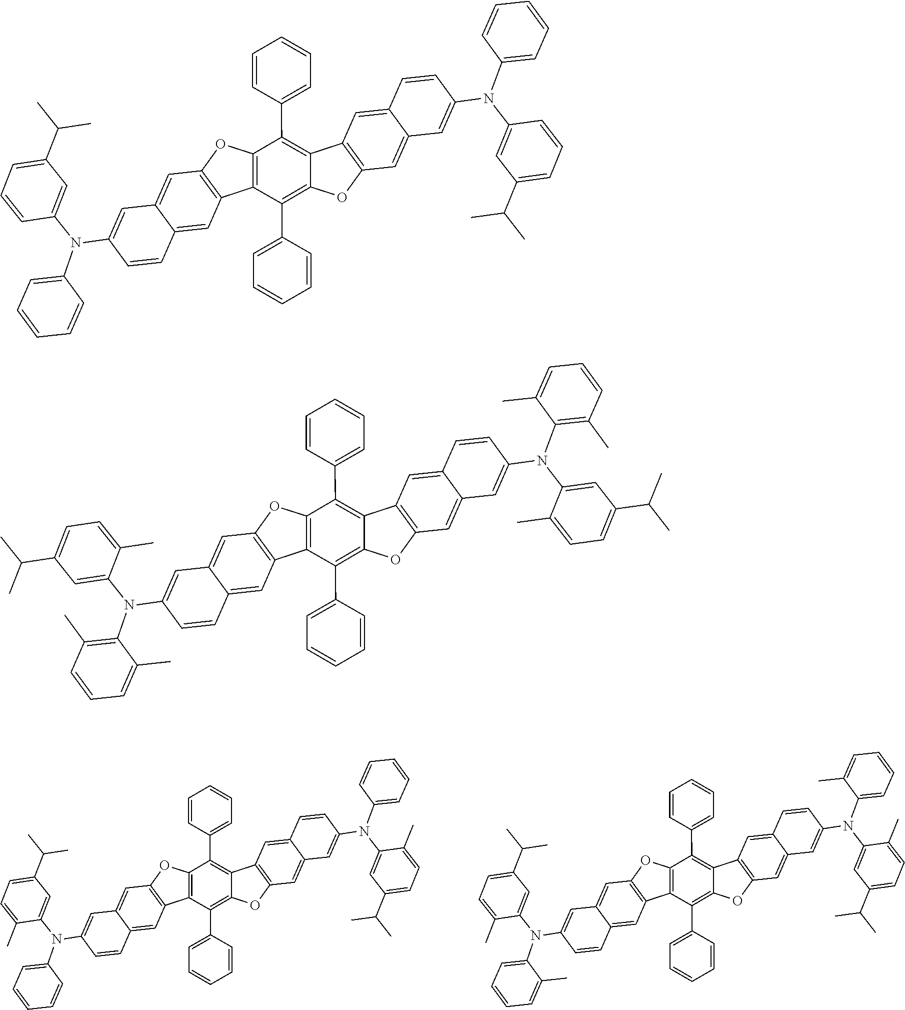

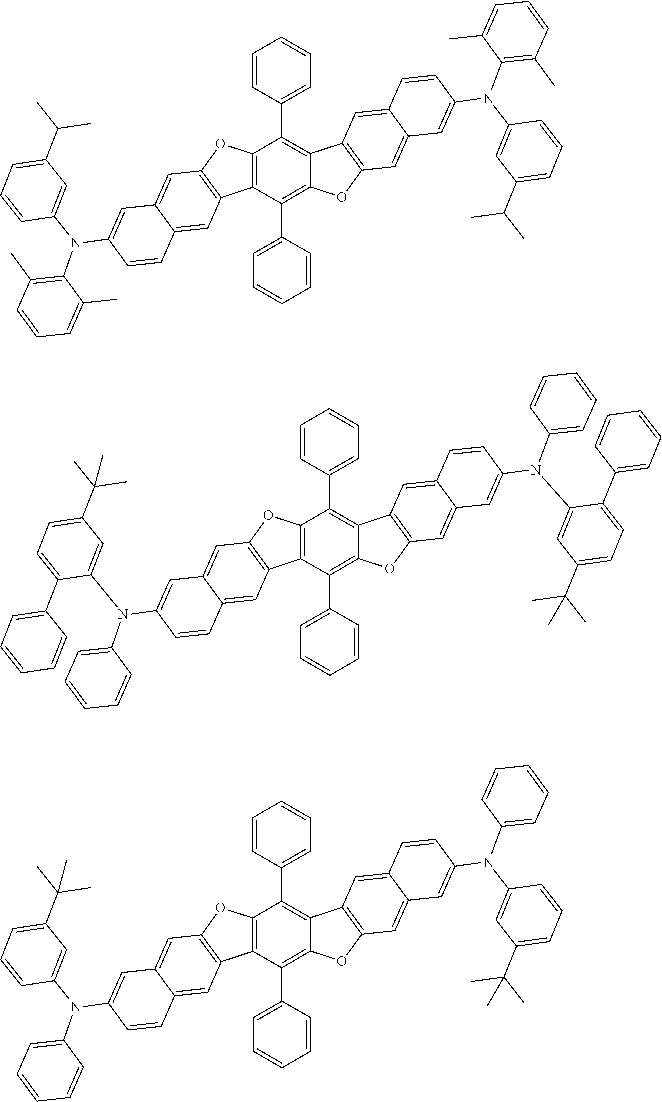 [SMILES: Cc1cccc(C)c1N(c1cccc(C(C)C)c1)c1ccc2cc3c(cc2c1)oc1c(-c2ccccc2)c2c(oc4cc5cc(N(c6cccc(C(C)C)c6)c6c(C)cccc6C)ccc5cc42)c(-c2ccccc2)c13 ; CC(C)(C)c1ccc(-c2ccccc2)c(N(c2ccccc2)c2ccc3cc4c(cc3c2)oc2c(-c3ccccc3)c3c(oc5cc6cc(N(c7ccccc7)c7cc(C(C)(C)C)ccc7-c7ccccc7)ccc6cc53)c(-c3ccccc3)c24)c1 ; CC(C)(C)c1cccc(N(c2ccccc2)c2ccc3cc4c(cc3c2)oc2c(-c3ccccc3)c3c(oc5cc6cc(N(c7ccccc7)c7cccc(C(C)(C)C)c7)ccc6cc53)c(-c3ccccc3)c24)c1]

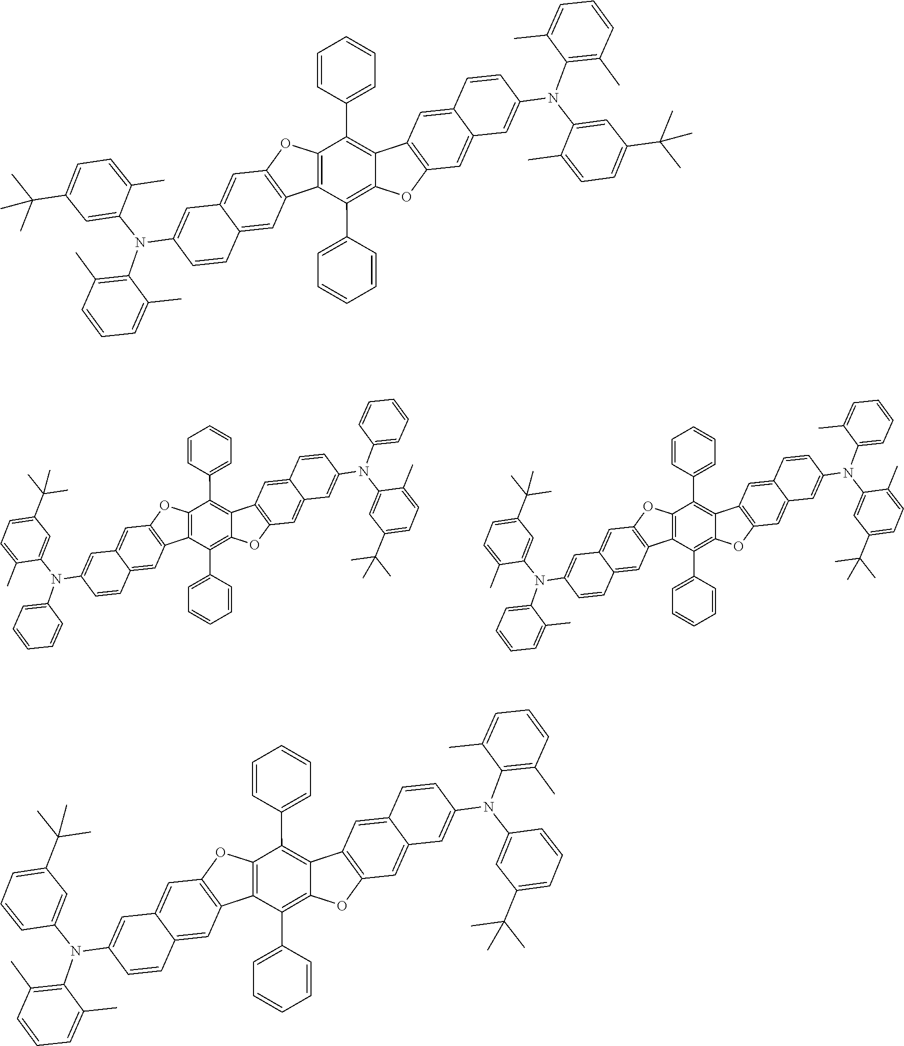

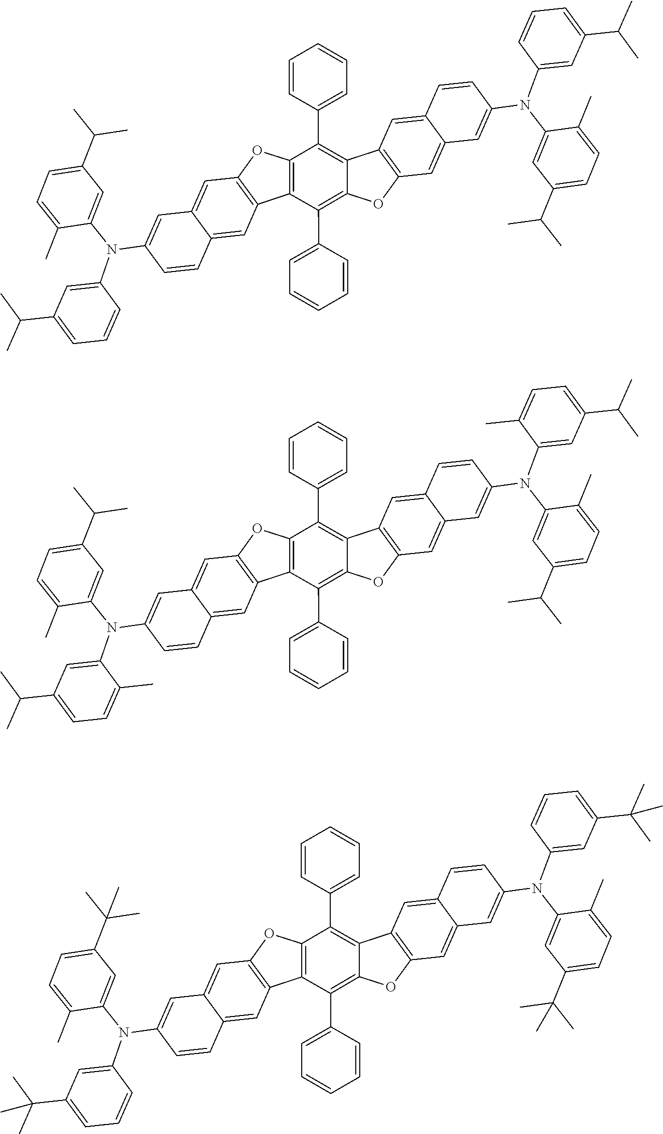

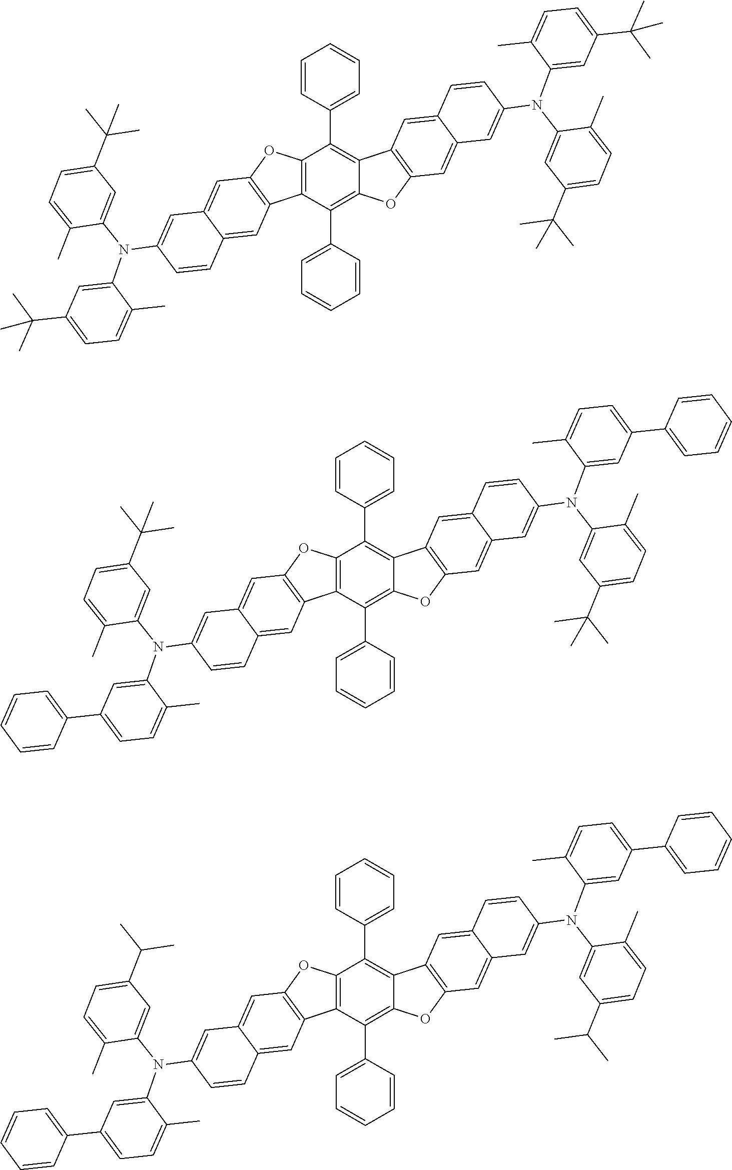 [SMILES: Cc1ccc(C(C)(C)C)cc1N(c1ccc2cc3c(cc2c1)oc1c(-c2ccccc2)c2c(oc4cc5cc(N(c6cc(C(C)(C)C)ccc6C)c6cc(C(C)(C)C)ccc6C)ccc5cc42)c(-c2ccccc2)c13)c1cc(C(C)(C)C)ccc1C ; Cc1ccc(-c2ccccc2)cc1N(c1ccc2cc3c(cc2c1)oc1c(-c2ccccc2)c2c(oc4cc5cc(N(c6cc(-c7ccccc7)ccc6C)c6cc(C(C)(C)C)ccc6C)ccc5cc42)c(-c2ccccc2)c13)c1cc(C(C)(C)C)ccc1C ; Cc1ccc(-c2ccccc2)cc1N(c1ccc2cc3c(cc2c1)oc1c(-c2ccccc2)c2c(oc4cc5cc(N(c6cc(-c7ccccc7)ccc6C)c6cc(C(C)C)ccc6C)ccc5cc42)c(-c2ccccc2)c13)c1cc(C(C)C)ccc1C]

The compound (1) can be synthesized, for example, in accordance with the synthesis process of Examples described later by using publicly known alternative reactions or materials corresponding to a target compound.

The compound (1) is useful as a material for an organic EL device.

The compound (1) is useful as a material of an emitting layer of the organic EL device, and is particularly useful as a fluorescent emitting material (also referred to as a fluorescent dopant) of the emitting layer.

The compound (1) is high in fluorescent quantum yield and can cause improvement in luminous efficiency of the obtained organic EL device by using as a material of the emitting layer.

The material for an organic EL device according to an aspect of the invention is characterized in that the material contains the compound (1).

The organic electroluminescence device according to an aspect of the invention contains:

a cathode,

an anode, and

at least one organic layer disposed between the cathode and the anode,

wherein at least one layer of the at least one organic layer contains the compound (1).

The organic electroluminescence device according to an aspect of the invention contains:

a cathode,

an anode, and

at least one organic layer of organic layer disposed between the cathode and the anode,

wherein at least one layer of the at least one organic layer contains the compound (1) as a fluorescent emitting material.

A luminous efficiency of the organic EL device can be improved by using the compound represented by the formula (1) for a predetermined organic layer, particularly for an emitting layer.

In one embodiment, the at least one organic layer contains an emitting layer, wherein the emitting layer contains the compound (1).

In the present specification, “at least one organic layer disposed between the cathode and the anode” refers to the layer when one organic layer is present between the cathode and the anode, and refers to at least one layer of the organic layers when there are a plurality of organic layers are present between the cathode and the anode.

Also, “at least one organic layer contains an emitting layer” means that, when one organic layer presents between the cathode and the anode, the layer is an emitting layer, and when a plurality of organic layers, at least one of the organic layers is an emitting layer.

In one embodiment, the organic EL device contains a hole-transporting layer between the anode and the emitting layer.

In one embodiment, the organic EL device contains an electron-transporting layer between the cathode and the emitting layer.

In the present specification, “at least one layer between emitting layer and anode” refers to the layerwhen one organic layer is present between the emitting layer and the anode, and refers to at least one layer of the organic when there is a plurality of organic layers are present between the emitting layer and the anode. For example, if there is two or more organic layers between the emitting layer and the anode, an organic layer closer to the emitting layer is called a “hole-transporting layer” and an organic layer closer to the anode is called a “hole-injecting layer.” A “hole-transporting layer” and a “hole-injecting layer” may be one layer, respectively; or may be two or more layers; or one may be one layer and the other may be two or more layers.

Similarly, “at least one layer between the emitting layer and the cathode” refers to a layer of organic layer between emitting layer and cathode, if present, or to at least one layer of organic layer, if present. For example, if there is two or more organic layers between the emitting layer and cathode, the organic layer closer to the emitting layer is called an “electron-transporting layer” and an organic layer closer to the cathode is called an “electron-injecting layer.” An “electron-transporting layer” and an “electron-injecting layer” may be one layer, respectively; or may be two or more layers, or one may be a layer and the other may be two or more layers.

In one embodiment, the emitting layer further contains a compound represented by the following formula (2) (hereinafter may be referred to as the compound (2)):

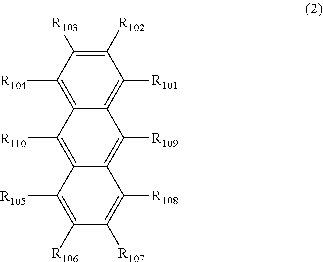

In the formula (2),

one or more pairs of adjacent two or more among R₁₀₁ to R₁₁₀ may form a substituted or unsubstituted, saturated or unsaturated ring;

R₁₀₁ to R₁₁₀ which do not form a substituted or unsubstituted, saturated or unsaturated ring are independently a hydrogen atom, a substituted or unsubstituted alkyl group including 1 to 50 carbon atoms, a substituted or unsubstituted alkenyl group including 2 to 50 carbon atoms, a substituted or unsubstituted alkynyl group including 2 to 50 carbon atoms, a substituted or unsubstituted cycloalkyl group including 3 to 50 carbon atoms, a substituted or unsubstituted alkoxy group including 1 to 50 carbon atoms, a substituted or unsubstituted alkylthio group including 1 to 50 carbon atoms, a substituted or unsubstituted aryloxy group including 6 to 50 carbon atoms, a substituted or unsubstituted arylthio group including 6 to 50 carbon atoms, a substituted or unsubstituted aralkyl group including 7 to 50 carbon atoms, Si(R₁₂₁)(R₁₂₂)(R₁₂₃),—C(═O)(R₁₂₄), —COOR₁₂₅, —N(R₁₂₅)(R₁₂₇), a halogen group, a cyano group, a nitro group, a substituted or an unsubstituted aryl group including 6 to 50 ring carbon atoms, a substituted or unsubstituted heterocyclic group including 5 to 50 ring atoms, or a group represented by the following formula (21);

wherein R₁₂₁ to R₁₂₇ are independently a hydrogen atom, a substituted or unsubstituted alkyl group including 1 to 50 carbon atoms, a substituted or unsubstituted aryl group including 6 to 50 ring carbon atoms, or a substituted or unsubstituted heterocyclic group including from 5 to 50 ring atoms; when two or more R₁₂₁ to R₁₂₇ are present, the two or more R₁₂₁ to R₁₂₇ may be the same or different;

provided that at least one R₁₀₁ to R₁₁₀ which do not form a substituted or unsubstituted, saturated or unsaturated ring is a group represented by the following formula (21); when two or more of the following formula (21) are present, two or more of the groups represented by the following formula (21) may be the same or different.

-L₁₀₁-Ar₁₀₁ (21)

wherein in the formula (21),

L₁₀₁ is a single bond, a substituted or unsubstituted arylene group including 6 to 30 ring carbon atoms or substituted or unsubstituted divalent heterocyclic group including 5 to 30 ring atoms; and

Ar₁₀₁ is a substituted or unsubstituted aryl group including 6 to 50 ring carbon atoms, or a substituted or unsubstituted heterocyclic group including from 5 to 50 ring atoms.

Specific examples of the substituent, the arbitrary substituent and the halogen atom of the compound (2) mentioned above are the same as those described above.

Hereinafter, “one or more pairs of adjacent two or more among R₁₀₁ to R₁₁₀ may form a substituted or unsubstituted, saturated or unsaturated ring” will be described.

The “one pair of” adjacent two or more among R₁₀₁ to R_{110″} is, for example, a combination of: R₁₀₁ and R₁₀₂; R₁₀₂ and R₁₀₃; R₁₀₃ and R₁₀₄; R₁₀₅ and R₁₀₆; R₁₀₇ and R₁₀₇; R₁₀₇ and R₁₀₈; R₁₀₁ and R₁₀₉; R₁₀₁, R₁₀₂, and R₁₀₃; and the like.

The substituent in “substituted” of “substituted or unsubstituted” for the saturated or unsaturated ring is the same as the arbitrary substituent in the formula (2).

The “saturated or unsaturated ring” means, when the ring is formed of R₁₀₁ and R₁₀₂, for example, a ring formed by a carbon atom with which R₁₀₁ is bonded, a carbon atom with which R₁₀₂ is bonded, and one or more arbitrary elements. Specifically, if a ring is formed by R₁₀₁ and R₁₀₂, when an unsaturated ring is formed by a carbon atom with which R₁₀₁ is bonded, a carbon atom with which R₁₀₂ is bonded, and four carbon atoms, the ring formed by R₁₀₁ and R₁₀₂ is a benzene ring.

The “arbitrary element” is preferably a C element, a N element, an O element, and a S element. In the arbitrary elements (a C element or a N element, for example), atomic bondings that do not form a ring may be terminated by a hydrogen atom, or the like.

The “one or more arbitrary element” is preferably 2 or more and 15 or less, more preferably 3 or more and 12 or less, and further preferably 3 or more and 5 or less arbitrary elements.

For example, R₁₀₁ and R₁₀₂ may forma ring, and simultaneously, R₁₀₅ and R₁₀₆ may form a ring. In this case, the compound represented by the formula (2) is a compound represented by the following formula (2A), for example.

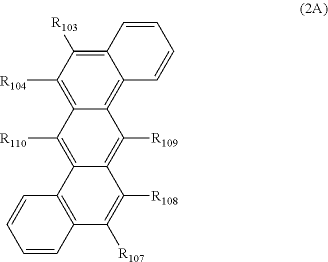

R₁₀₁ to R₁₁₀ are preferably independently a hydrogen atom, a substituted or unsubstituted alkyl group including 1 to 50 carbon atoms, a substituted or unsubstituted aryl group including 6 to 50 ring carbon atoms, a substituted or unsubstituted heterocyclic group including 5 to 50 ring or a group represented by the formula (21).

More preferably, R₁₀₁ to R₁₁₀ are independently a hydrogen atom, a substituted or unsubstituted aryl group including 6 to 50 ring carbon atoms, a substituted or unsubstituted heterocyclic group including from 5 to 50 ring atoms, or a group represented by the formula (21).

Still more preferably, R₁₀₁ to R₁₁₀ are independently a hydrogen atom, a substituted or unsubstituted aryl group including 6 to 18 ring carbon atoms, a substituted or unsubstituted heterocyclic group including 5 to 18 ring atoms, or a group represented by the formula (21).

In one embodiment, it is preferable that at least one of R₁₀₉ and R₁₁₀ be a group represented by the formula (21).

In one embodiment, it is preferable that R₁₀₉ and R₁₁₀ be independently a group represented by the formula (21).

In one embodiment, the compound represented by the formula (2) (hereinafter may be referred to as a compound (2)) is a compound represented by the following formula (2-1).

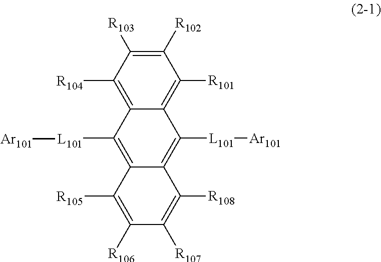

In the formula (2-1), R₁₀₁ to R₁₀, L₁o and Ar₁o are as defined in the formula (2).

In one embodiment, the compound (2) is a compound represented by the following formula (2-2).

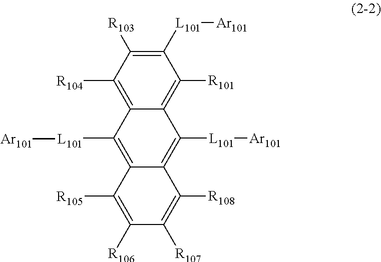

In the formula (2-2), R₁₀₁, R₁₀₃ to R₁₀₆, L₁₀₁ and Ar₁₀₁ are as defined in the formula (2).

In one embodiment, the compound (2) is a compound represented by the following formula (2-3).

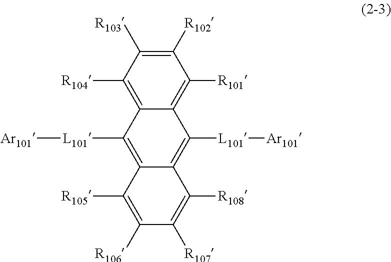

In the formula (2-3),

R_{101′} to R_{106′} are independently a hydrogen atom or a substituted or unsubstituted aryl group including 6 to 50 ring carbon atoms;

L_{101′} is a substituted or unsubstituted arylene group including 6 to 50 ring carbon atoms; two L₁₀₁s may be the same or different; and

Ar_{101′} is a substituted or unsubstituted aryl group including 6 to 50 ring carbon atoms; two Ar₁₀₁s may be the same or different.

In one embodiment, the compound (2) is a compound represented by the following formula (2-4).

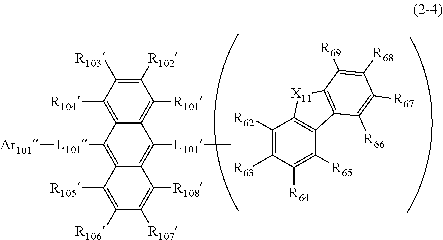

In the formula (2-4),

R_{101′} to R_{106′} are independently a hydrogen atom or a substituted or unsubstituted aryl group including 6 to 50 ring carbon atoms;

L_{101′} is a substituted or unsubstituted arylene group including 6 to 50 ring carbon atoms;

L_{101″} is a substituted or unsubstituted arylene group including 6 to 50 ring carbon atoms or a substituted or unsubstituted divalent heterocyclic group including 5 to 50 ring atoms;

Ar_{101″} is a substituted or unsubstituted aryl group including 6 to 50 ring carbon atoms, or a substituted or unsubstituted monovalent heterocyclic group including 5 to 50 ring atoms;

X₁₁ is O, S, or N(R₆₁);

R₆₁ is a hydrogen atom, a substituted or unsubstituted alkyl group including 1 to 50 carbon atoms or a substituted or unsubstituted aryl group including 6 to 50 ring carbon atoms; one of R₆₂ to R₆₉ is bonded with L₁₀₁;

R₆₂ to R₆₉ which do not bonded with L_{101′} are independently a hydrogen atom, a substituted or unsubstituted alkyl group including 1 to 50 carbon atoms or a substituted or unsubstituted aryl group including 6 to 50 ring carbon atoms; and one or more pairs adjacent two or more among R₆₂ to R₆₉ which do not bonded with L_{101′} may be bonded with each other to form a substituted or unsubstituted, saturated or unsaturated ring.

In one embodiment, the compound represented by the formula (2) is represented by the following formula (2-4A).

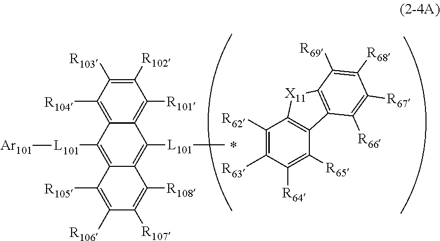

In the formula (2-4A),

L₁₀₁ and Ar₁₀₁ are as defined in the formula (2);

R_{101′} to R_{106′0} are independently a hydrogen atom or a substituted or unsubstituted aryl group including 6 to 50 ring carbon atoms;

X₁₁ is O, S, or N(R₆₁);

R₆₁ is a hydrogen atom, a substituted or unsubstituted alkyl group including 1 to 50 carbon atoms or a substituted or unsubstituted aryl group including 6 to 50 ring carbon atoms; one or more pairs adjacent two or more among R_{62′} to R₆₉, may form a substituted or unsubstituted, saturated or unsaturated ring, and adjacent two among R_{62′} to R_{69′} form a ring represented by the following formula (2-4A-1);

R_{62′} to R_{69′} which do not form a substituted or unsubstituted, saturated or unsaturated ring is independently a hydrogen atom, a substituted or unsubstituted alkyl group including 1 to 50 carbon atoms, or a substituted or unsubstituted aryl group including 6 to 50 ring carbon atoms.

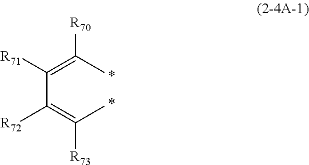

In the formula (2-4A-1),

two of * are bonded with two adjacent among R_{62′} to R_{69′};

one of R₇₀ to R₇₃ is bonded with L₁₀₁; and

R₇₀ to R₇₃ which are not bonded with L₁₀₁ are independently a hydrogen atom, a substituted or unsubstituted alkyl group including 1 to 50 carbon atoms or a substituted or unsubstituted aryl group including 6 to 50 ring carbon atoms.

In one embodiment, the compound (2) is selected from the group consisting of compounds represented by the following formulas (2-5-1) to (2-5-3).

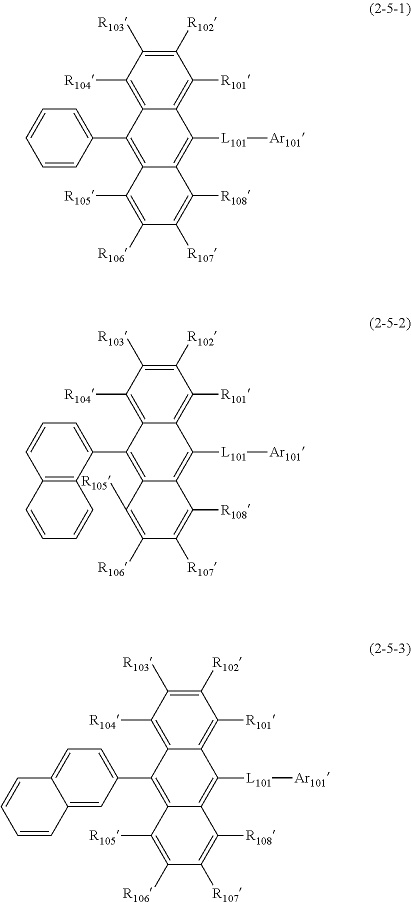

In the formulas (2-5-1) to (2-5-3), L₁₀₁ is as defined in the formula (2), and R_{101′} or to R_{106′} and Ar_{101′} are as defined in the formula (2-3).

In one embodiment, the compound represented by the formulas (2-5-1) to (2-5-3) is a compound represented by the following formula (2-5-1H) to (2-5-3H).

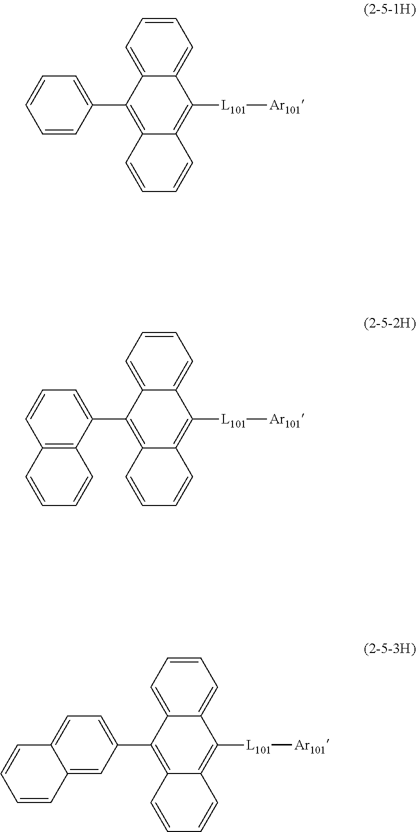

In the formula (2-5-1H) to (2-5-3H), L₁₀₁ is as defined in the formula (2), and Ar_{101′} is as defined in the formula (2-3).

In one embodiment, the compound (2) is a compound represented by the following formula (2-6).

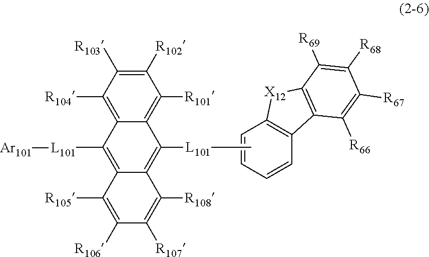

In the formula (2-6), L₁₀₁ and Ar₁₀₁ are as defined in the formula (2);

R_{101′} to R_{106′} are as defined in the formula (2-4);

R₆₆ to R₆₉ are as defined in the formula (2-4); provided that R₆₆ and R₆₇, R₆₇ and R₆₈, and R₆₉ and R₆₇ are not bonded with each other to form a ring; and

X₁₂ is O or S.

In one embodiment, the compound represented by the formula (2-6) is a compound represented by the following formula (2-6H).

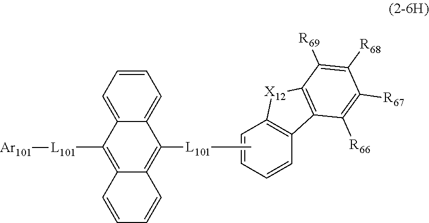

In the formula (2-6H), L₁₀₁ and Ar₁₀₁ are as defined in the formula (2);

R₆₆ to R₆₉ are as defined in the formula (2-4); provided that R₆₆ and R₆₇, R₆₇ and R₆₈, and R₆₉ and R₆₇ are not bonded with each other to form a ring; and

X₁₂ is O or S.

In one embodiment, the compound represented by the formula (2-6H) is a compound represented by the following formula (2-6Ha).

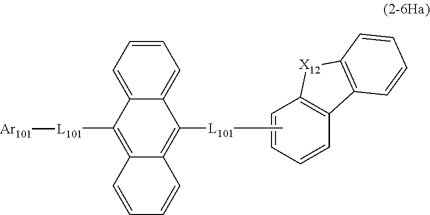

In the formula (2-6Ha), L₁₀₁ and Ar₁₀₁ are as defined in the formula (2); and

X₁₂ is as defined in the formula (2-6).

In one embodiment, the compound represented by the formula (2-6Ha) is a compound represented by the following formula (2-6Hb-1) or (2-6Ha-2).

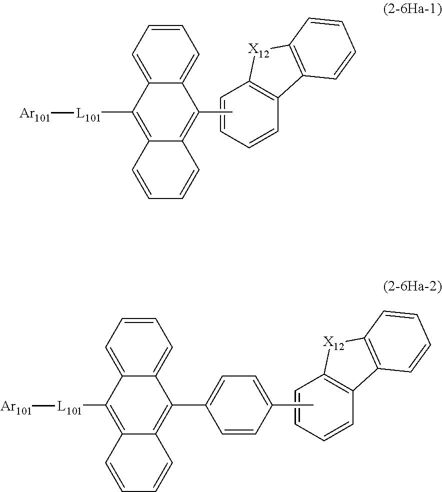

In the formulas (2-6Ha-1) and (2-6Ha-2), L₁₀ and Ar₁₀ are as defined in the formula (2); and X₁₂ is as defined in the formula (2-6).

In one embodiment, the compound (2) is a compound represented by the following formula (2-7).

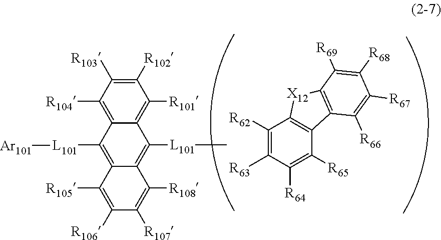

In the formula (2-7), L₁₀ and Ar₁₀ are as defined in the formula (2); R_{101′} to R_{106′} are independently a hydrogen atom or a substituted or unsubstituted aryl group including 6 to 50 ring carbon atoms;

one of R₆₂ to R₆₉ is bonded with L₁₀₁; and

R₆₂ to R₆₉ which are not bonded with L₁₀₁ is independently a hydrogen atom, a substituted or unsubstituted alkyl group including 1 to 50 carbon atoms or a substituted or unsubstituted aryl group including 6 to 50 ring carbon atoms; provided that one pair among R₆₆ and R₆₇, R₆₇ and R₆₈, and R₆₈ and R₆₉ is bonded with each other to form a substituted or unsubstituted, saturated or unsaturated ring. Preferably, one pair among R₆₆ and R₆₇, R₆₇ and R₆₈, and R₆₈ and R₆₉ is bonded with each other to form an unsubstituted benzene ring.

In one embodiment, the compound represented by the formula (2-7) is a compound represented by the following formula (2-7H).

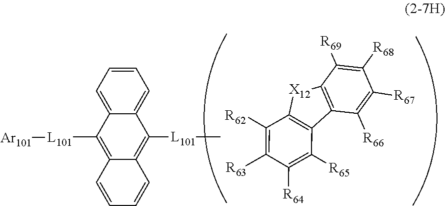

In the formula (2-7H), L₁₀₁ and Ar₁₀₁ are as defined in the formula (2); and

R₆₂ to R₆₉ are as defined in the formula (2-7); provided that one pair among R₆₆ and R₆₇, R₆₇ and R₆₈, and R₆₈ and R₆₉ is bonded with each other to form a substituted or unsubstituted, saturated or unsaturated ring. Preferably, one pair among R₆₆ and R₆₇, R₆₇ and R₆₈, and R₆₈ and R₆₉ are bonded with each other to form an unsubstituted benzene ring.

In one embodiment, the compound (2) is a compound represented by the following formula (2-8).

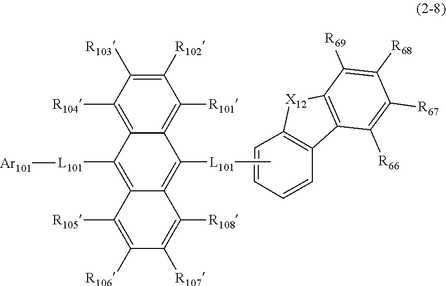

In the formula (2-8), L₁₀₁ and Ar₁₀₁ are as defined in the formula (2);

R_{101′} to R_{108′} are as defined in the formula (2-7); and

R₆₆ to R₆₉ are independently a hydrogen atom, a substituted or unsubstituted alkyl group including 1 to 50 carbon atoms or a substituted or unsubstituted aryl group including 6 to 50 ring carbon atoms; provided that one pair among R₆₆ and R₆₇, R₆₇ and R₆₈, or R₆₈ and R₆₉ is bonded with each other to form a substituted or unsubstituted, saturated or unsaturated ring. Preferably, one pair among R₆₆ and R₆₇, R₆₇ and R₆₈, or R₆₈ and R₆₉ is bonded each otherto form an unsubstituted benzene ring.

In one embodiment, the compound represented by the formula (2-8) is a compound represented by the following formula (2-8H).

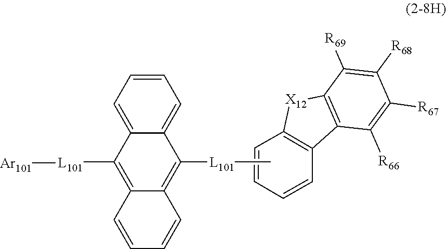

In the formula (2-8H), L₁₀₁ and Ar₁₀₁ are as defined in the formula (2); and

R₆₆ to R₆₉ are as defined in the formula (2-8); provided that one pair among Re and R₆₇, R₆₇ and R₆₈, or R₆₈ and R₆₉ is bonded with each other to form a substituted or unsubstituted, saturated or unsaturated ring. Preferably, one pair among R₆₆ and R₆₇, R₆₇ and R₆₈, or R₆₈ and R₆₉ is bonded with each other to form an unsubstituted benzene ring.

In one embodiment, one pair among R₆₆ and R₆₇, R₆₇ and R₆₆, and R₆₈ and R₆₉ is bonded with each other to form a ring represented by the following formula (2-8-1) or (2-8-2); and

R₆₆ to R₆₉ which do not form a ring represented by the formula (2-8-1) or (2-8-2) do not form a substituted or unsubstituted, saturated or unsaturated ring.

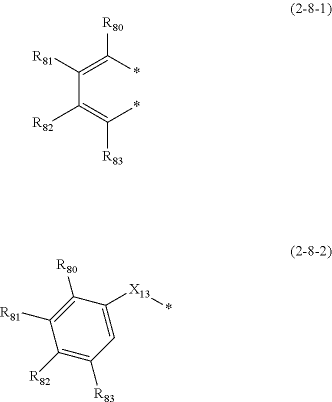

In the formulas (2-8-1) and (2-8-2),

two of* are independently bonded with one pair among R₆₆ and R₆₇, R₆₇ and R₆₈, or R₆₈ and R₆₉;

R₈₀ to R₈₃ are independently a hydrogen atom, a substituted or unsubstituted alkyl group including 1 to 50 carbon atoms or a substituted or unsubstituted aryl group including 6 to 50 ring carbon atoms; and

X₁₃ is O or S.

In one embodiment, the compound represented by the formula (2) is represented by the following formula (2-9).

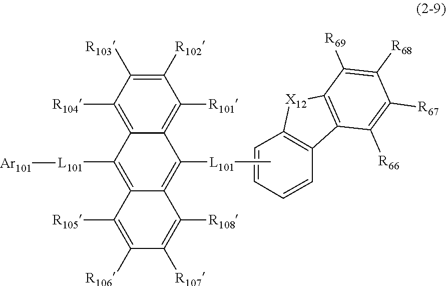

In the formula (2-9),

L₁₀₁ and Ar₁₀₁ are as defined in the formula (2);

R_{101′} to R_{106′} are as defined in the formula (2-4);

R₆₆ to R₆₉ are as defined in the formula (2-4); provided that R₆₆ and R₆₇, R₆₇ and R₆₆, and R₆₉ and R₆₇ are not bonded with each other to form a substituted or unsubstituted, saturated or unsaturated ring; and

X₁₂ is O or S.

In one embodiment, the compound (2) is selected from the group consisting of compounds represented by the following formulas (2-9-1) to (2-9-4).

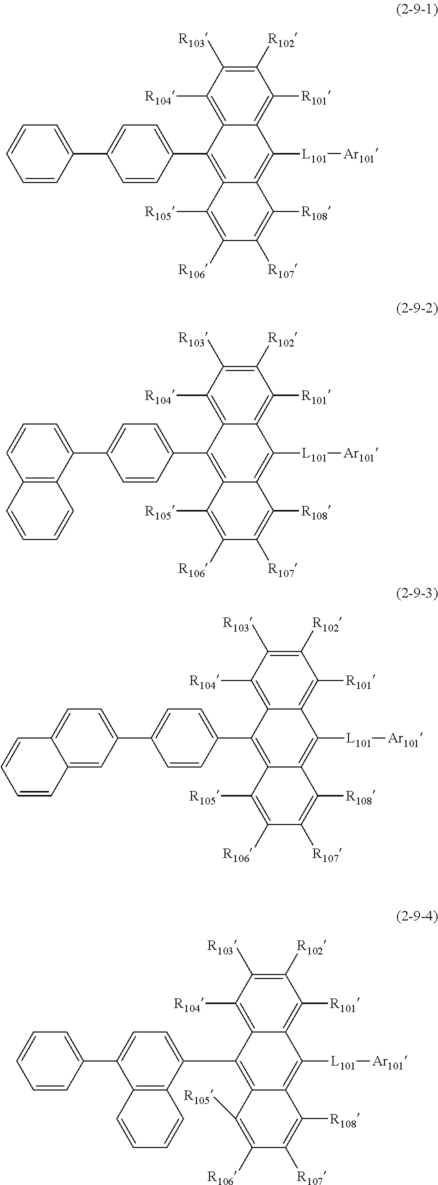

In the formulas (2-9-1) to (2-9-4), L₁₀ is as defined in the formula (2); and

Ar_{101′} and R_{101′} to R_{106′} are as defined in the formula (2-4).

In one embodiment, the compound represented by the formulas (2-9-1) to (2-9-4) are compounds represented by the following formula (2-9-1H) to (2-9-4H), respectively.

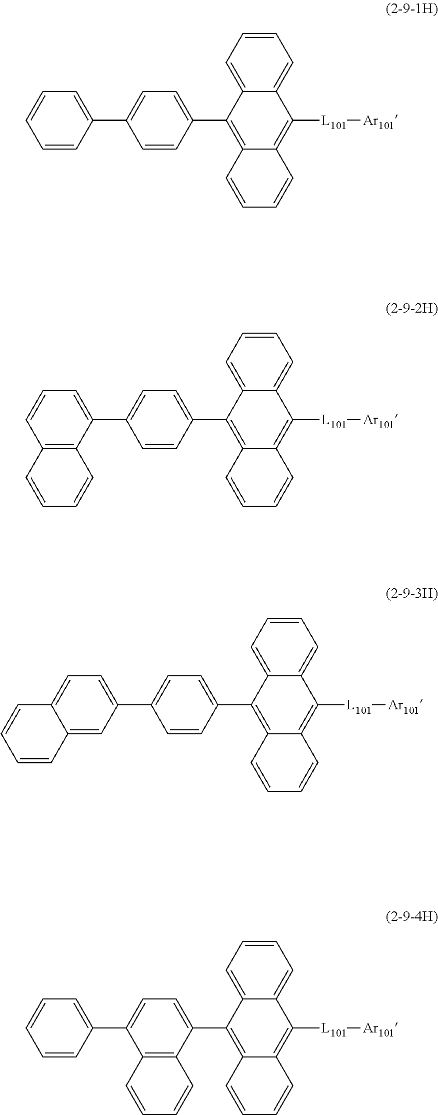

In the formulas (2-9-1H) to (2-9-4H), L₁₀₁ is as defined in the formula (2); and

Ar_{101′} is a substituted or unsubstituted aryl group including 6 to 50 ring carbon atoms; two Ar₁₀₁s may be the same or different.

Specific examples of the compound represented by the formula (2) include the compounds shown below.

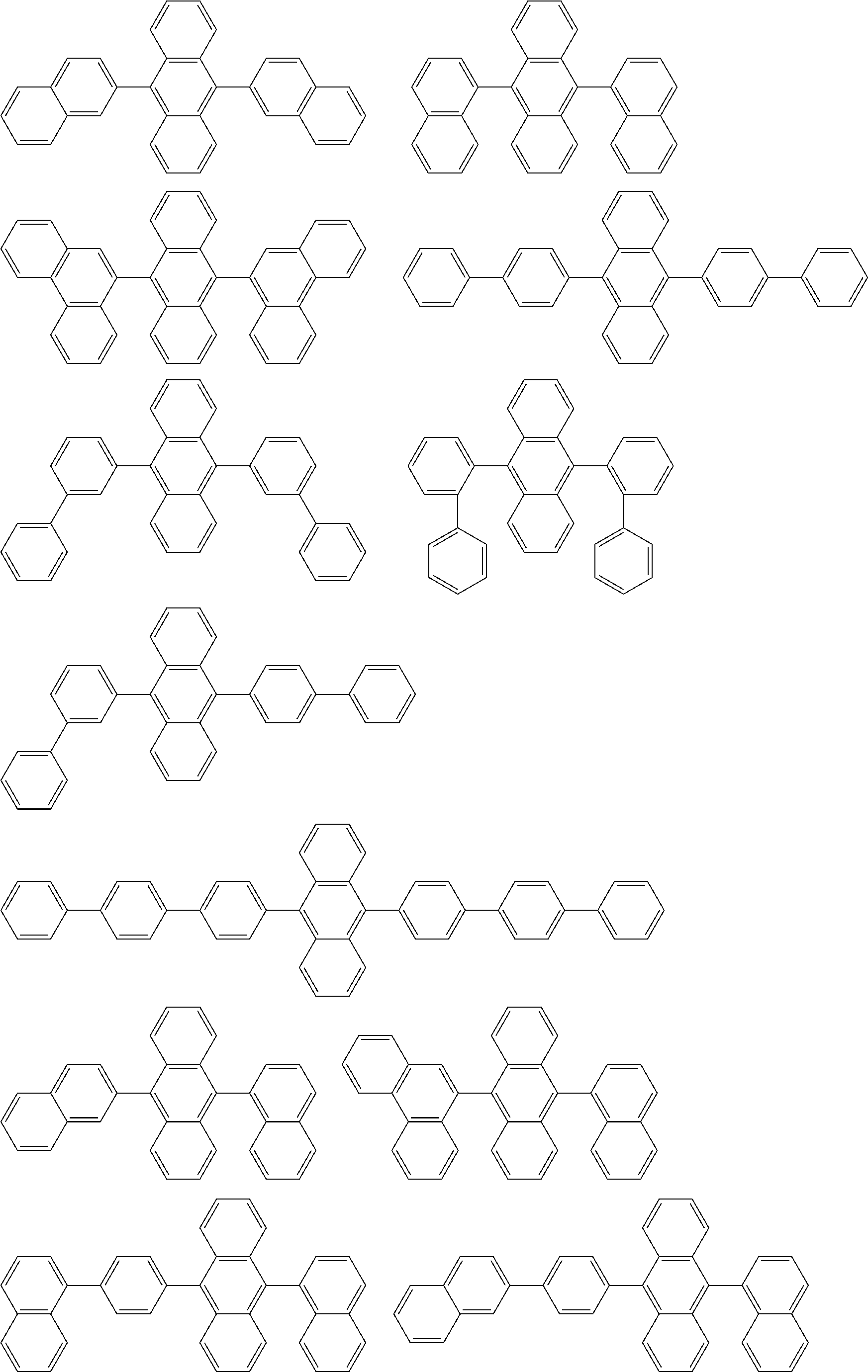

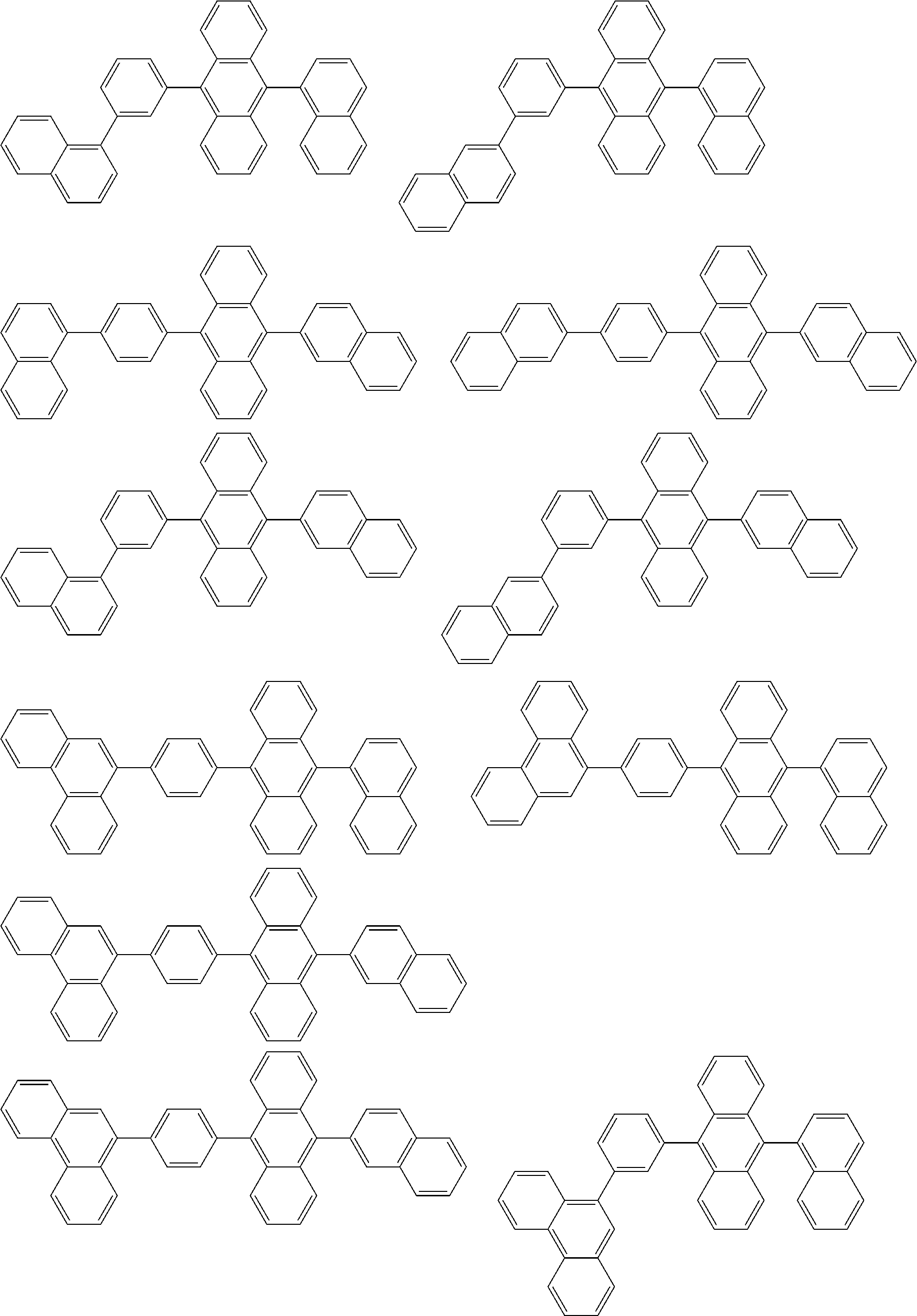

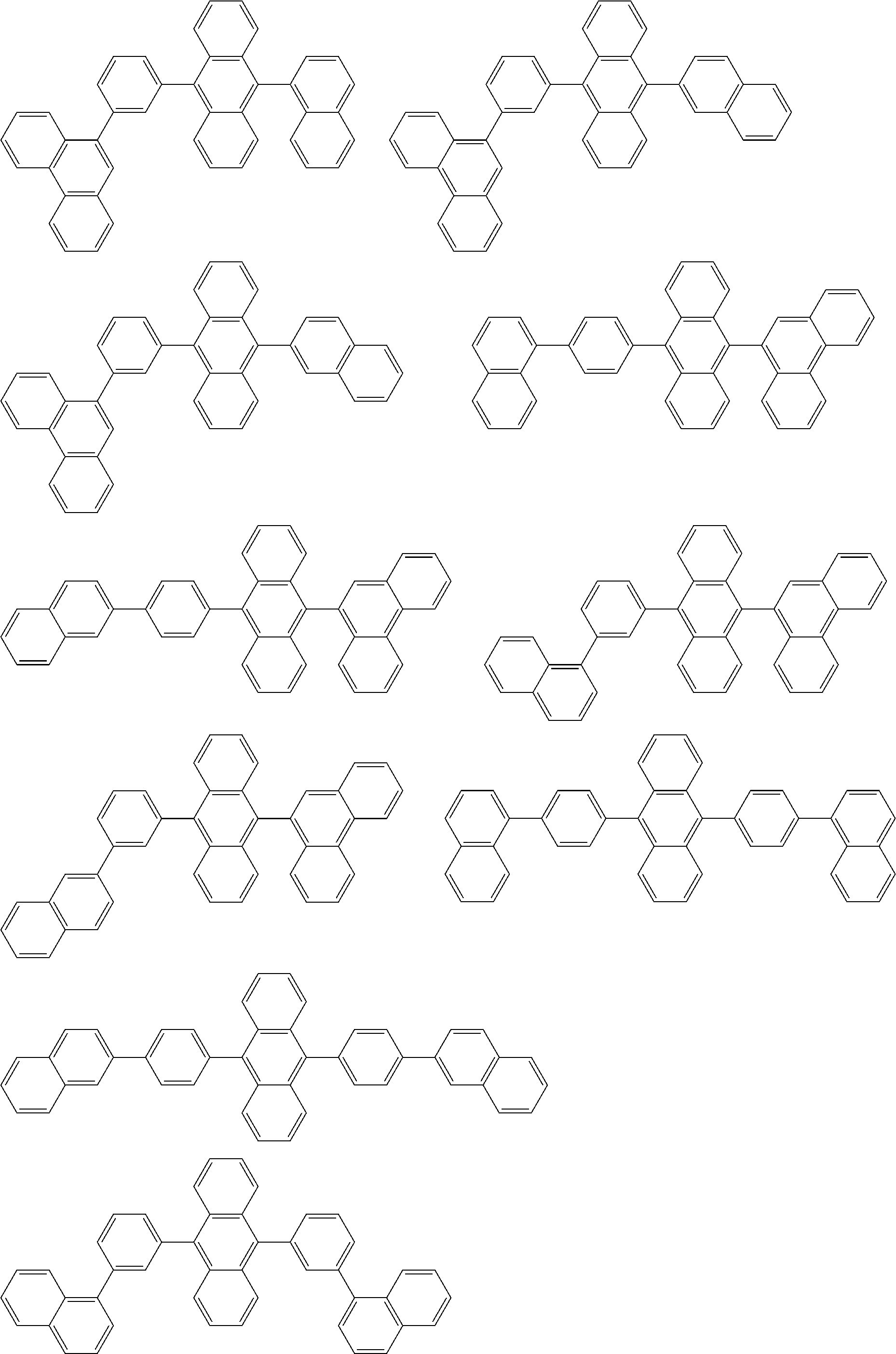

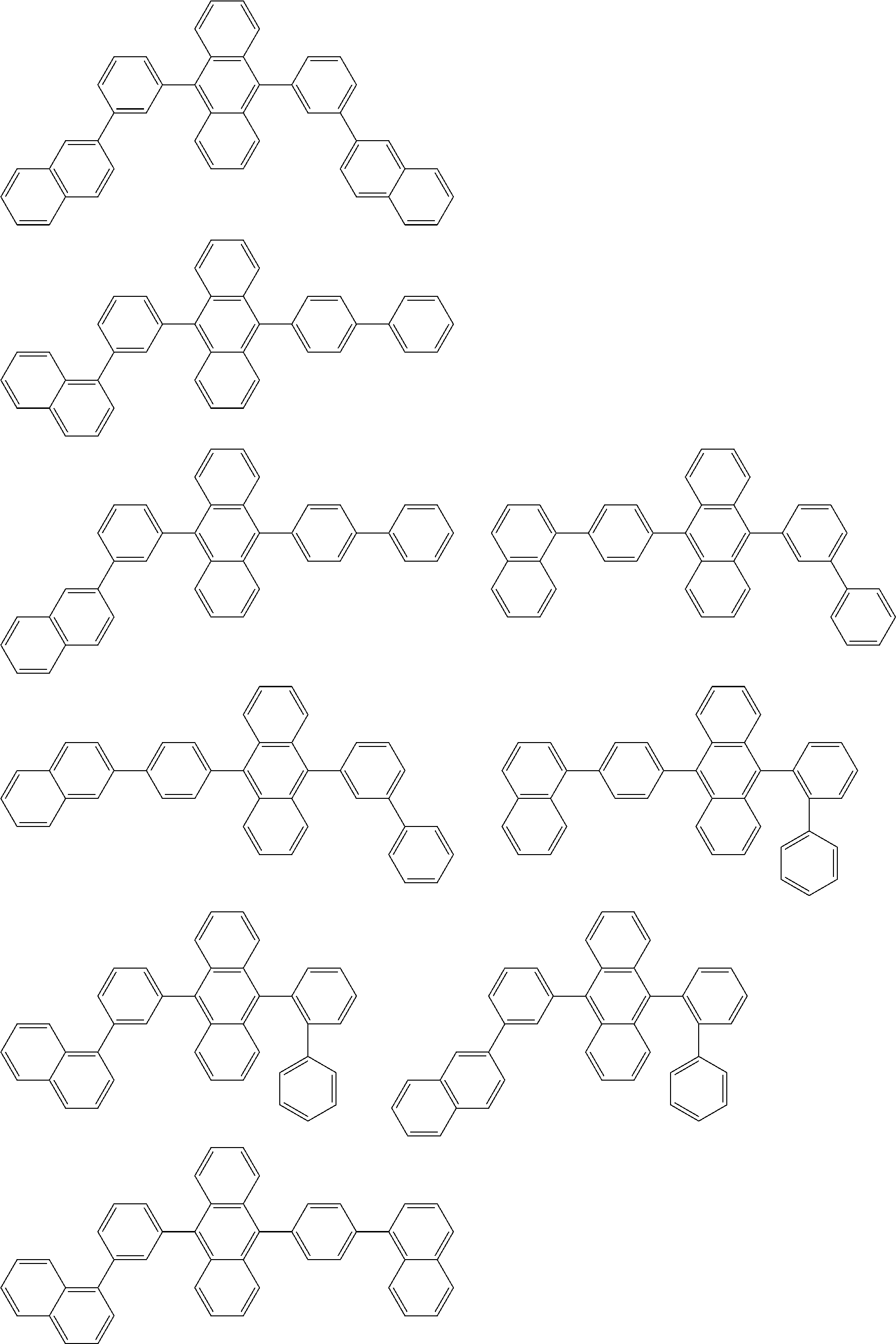

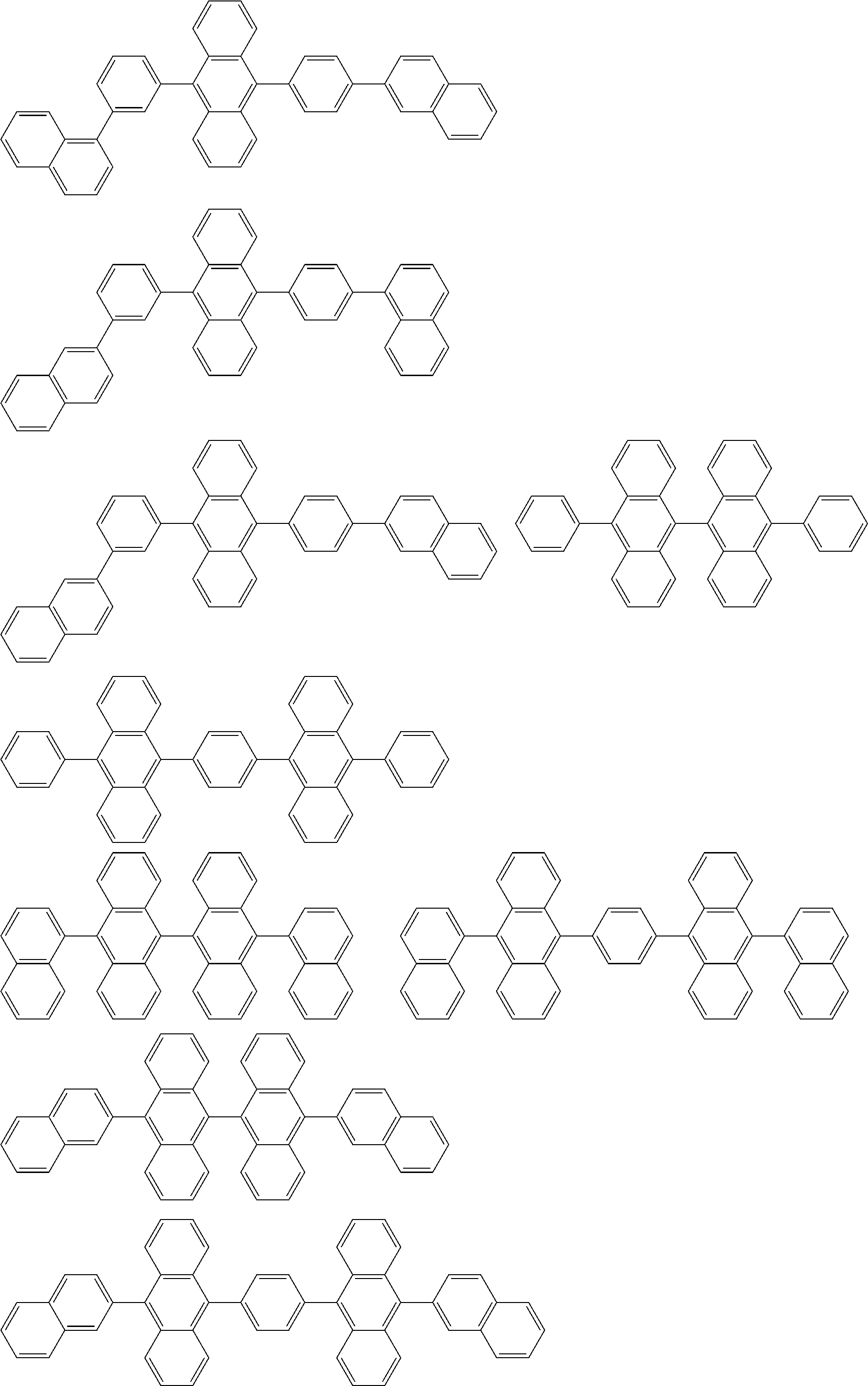

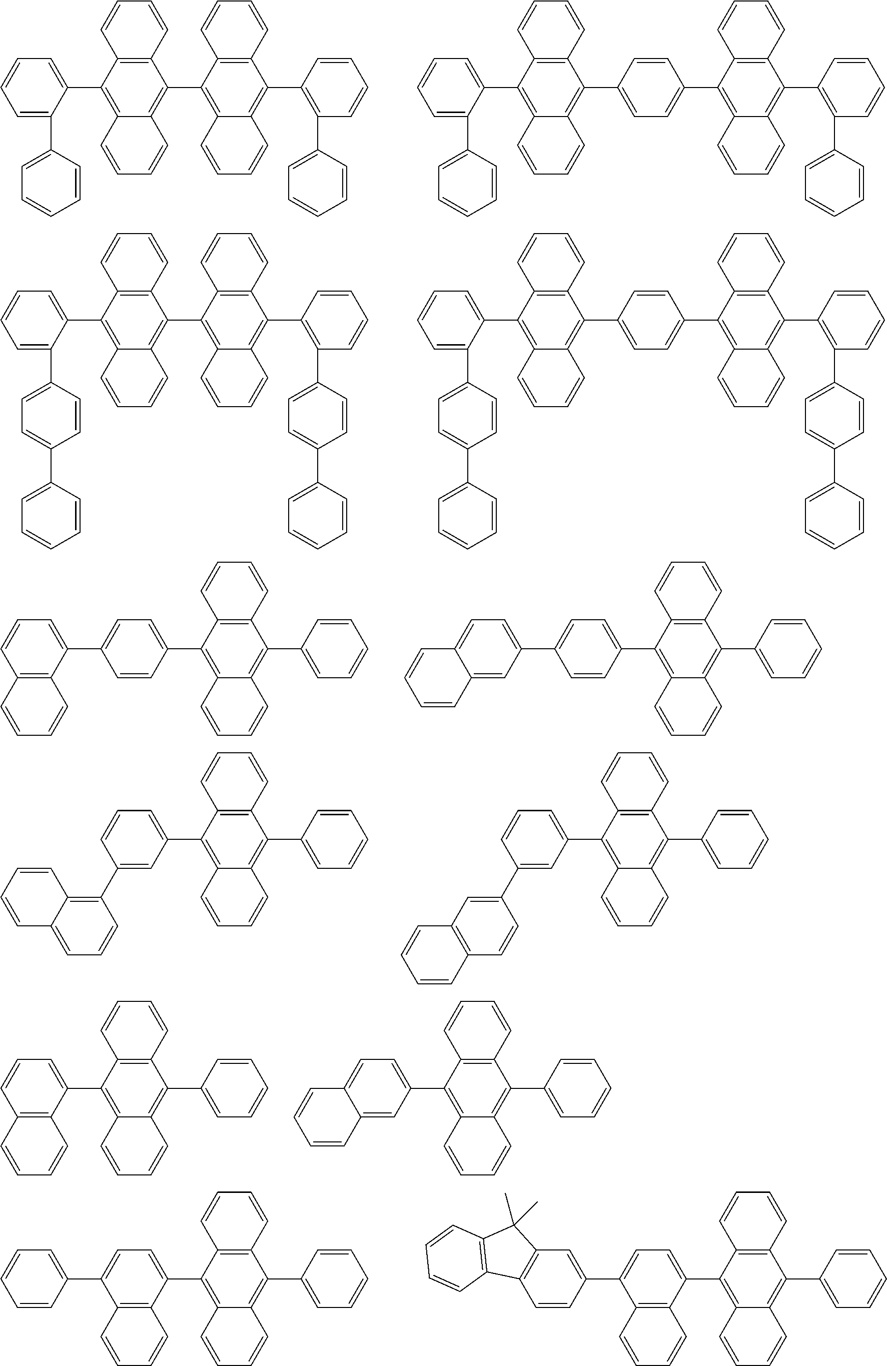

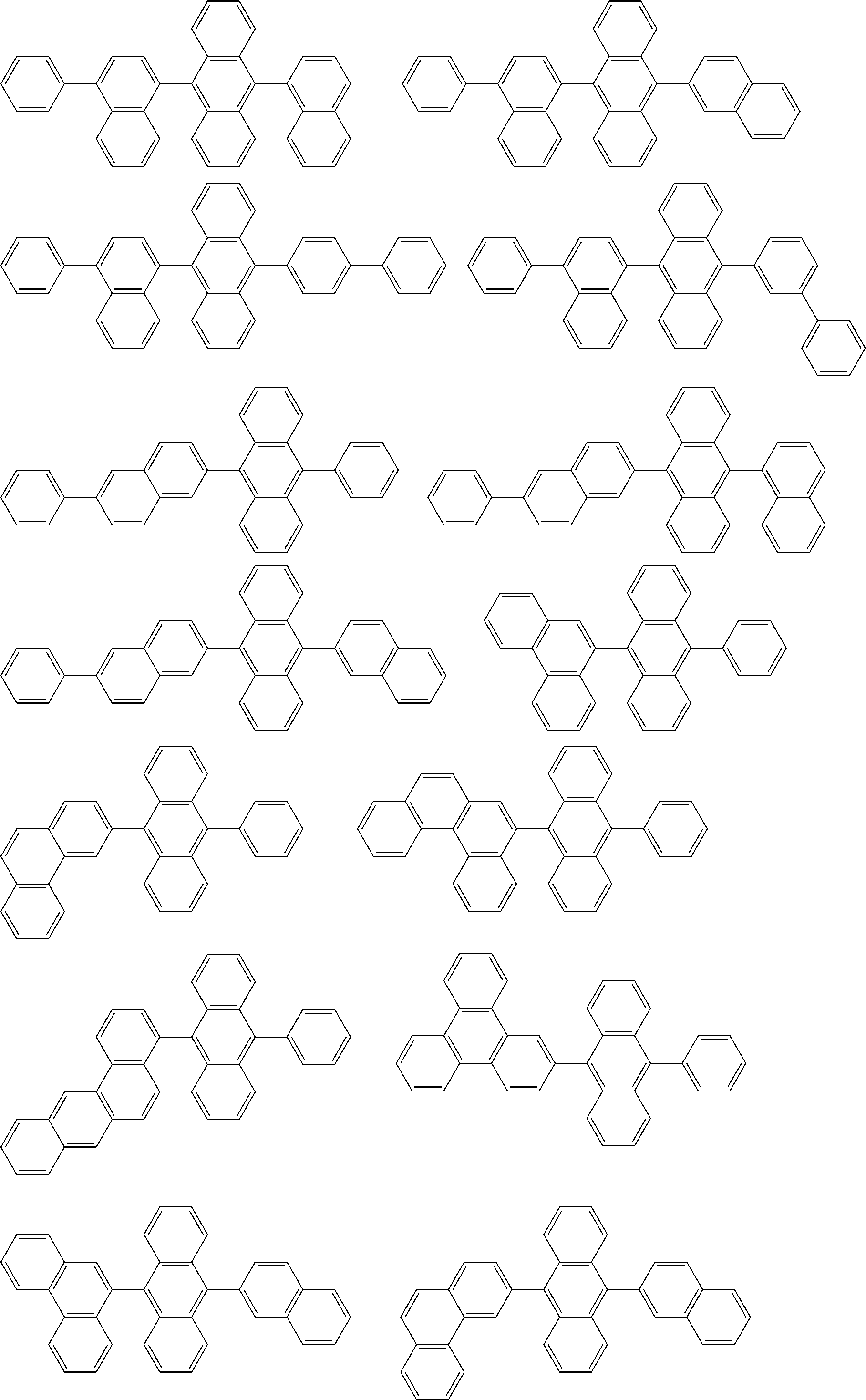

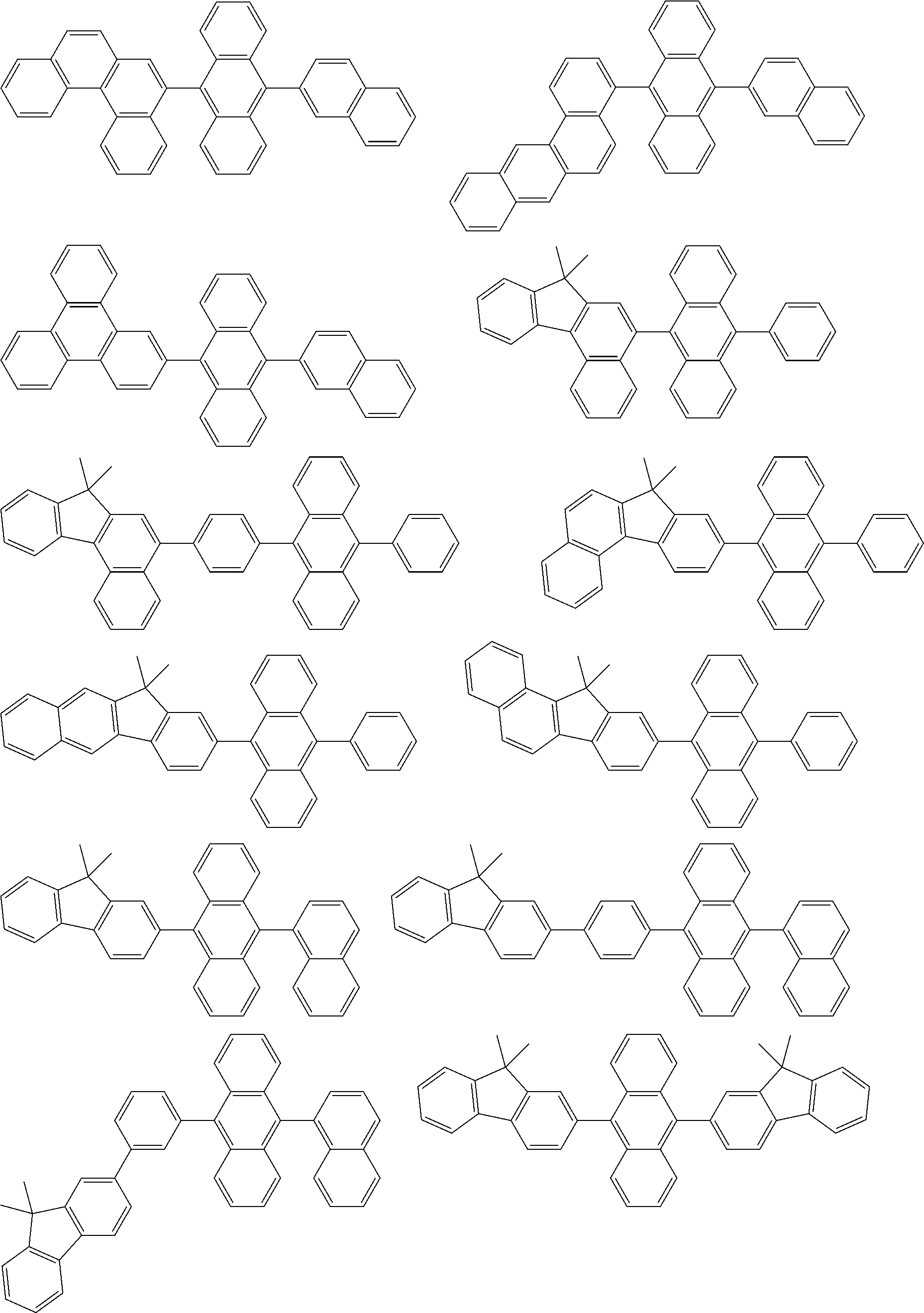

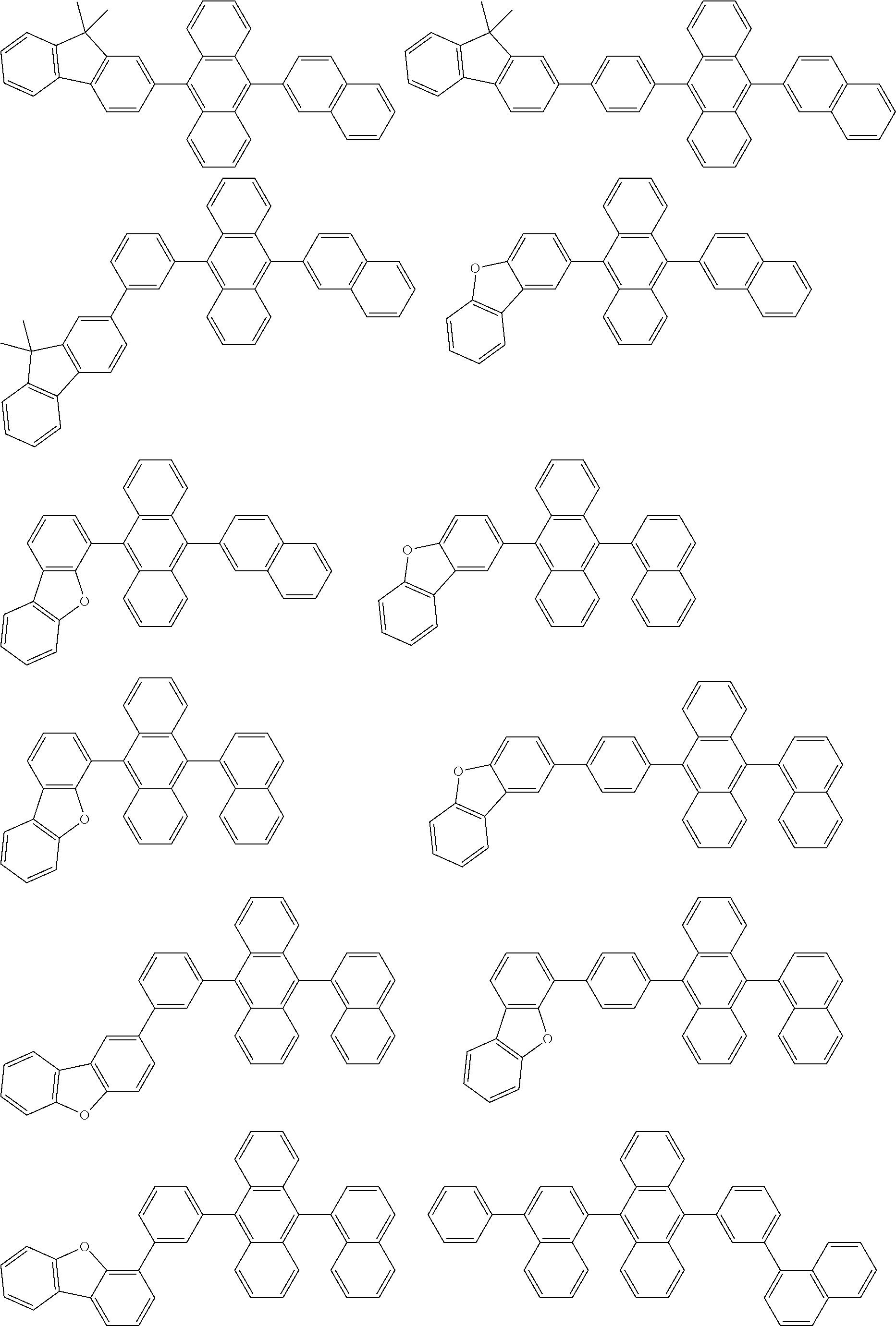

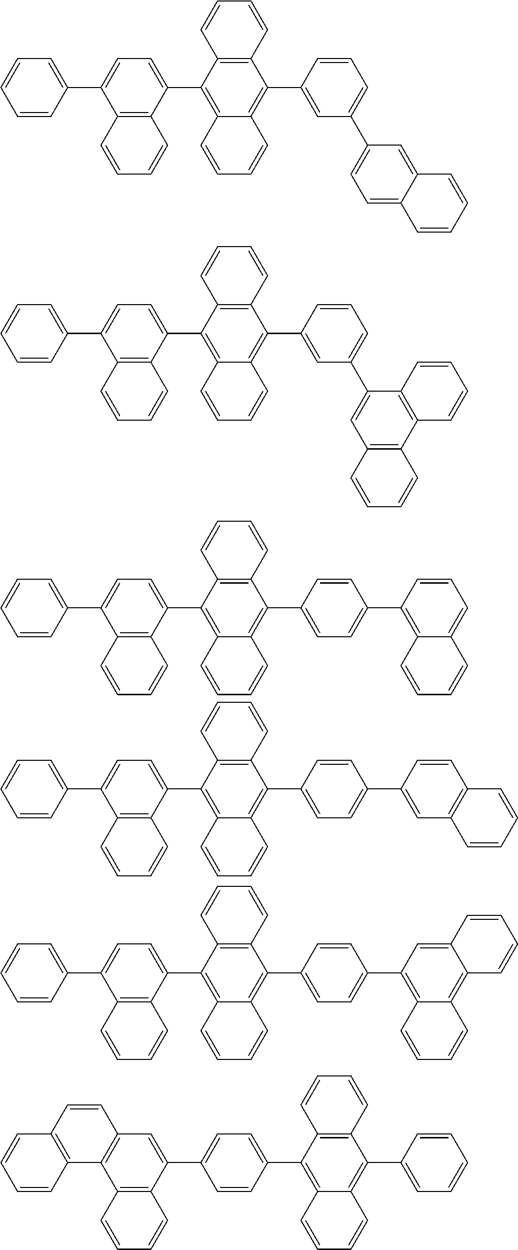

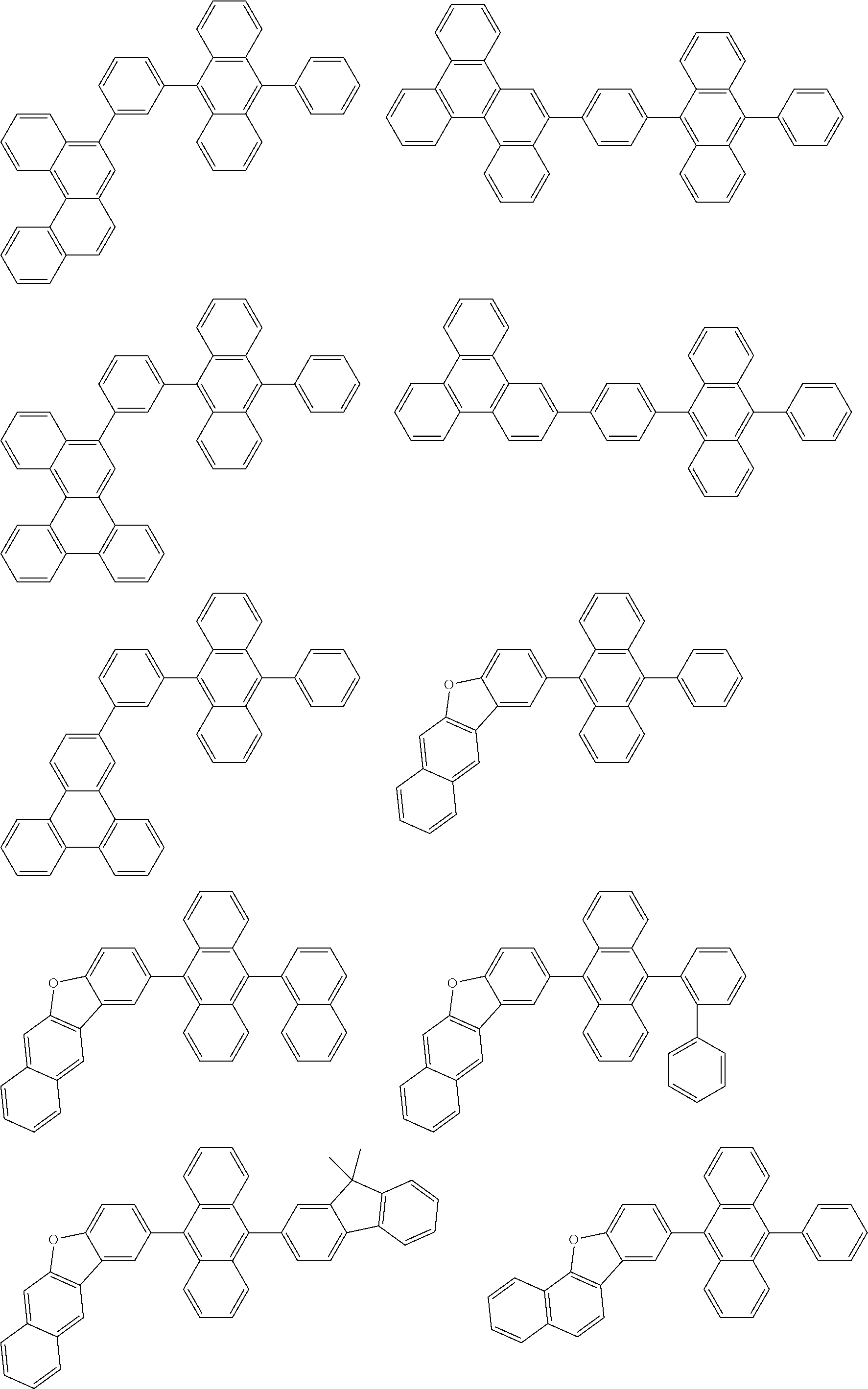

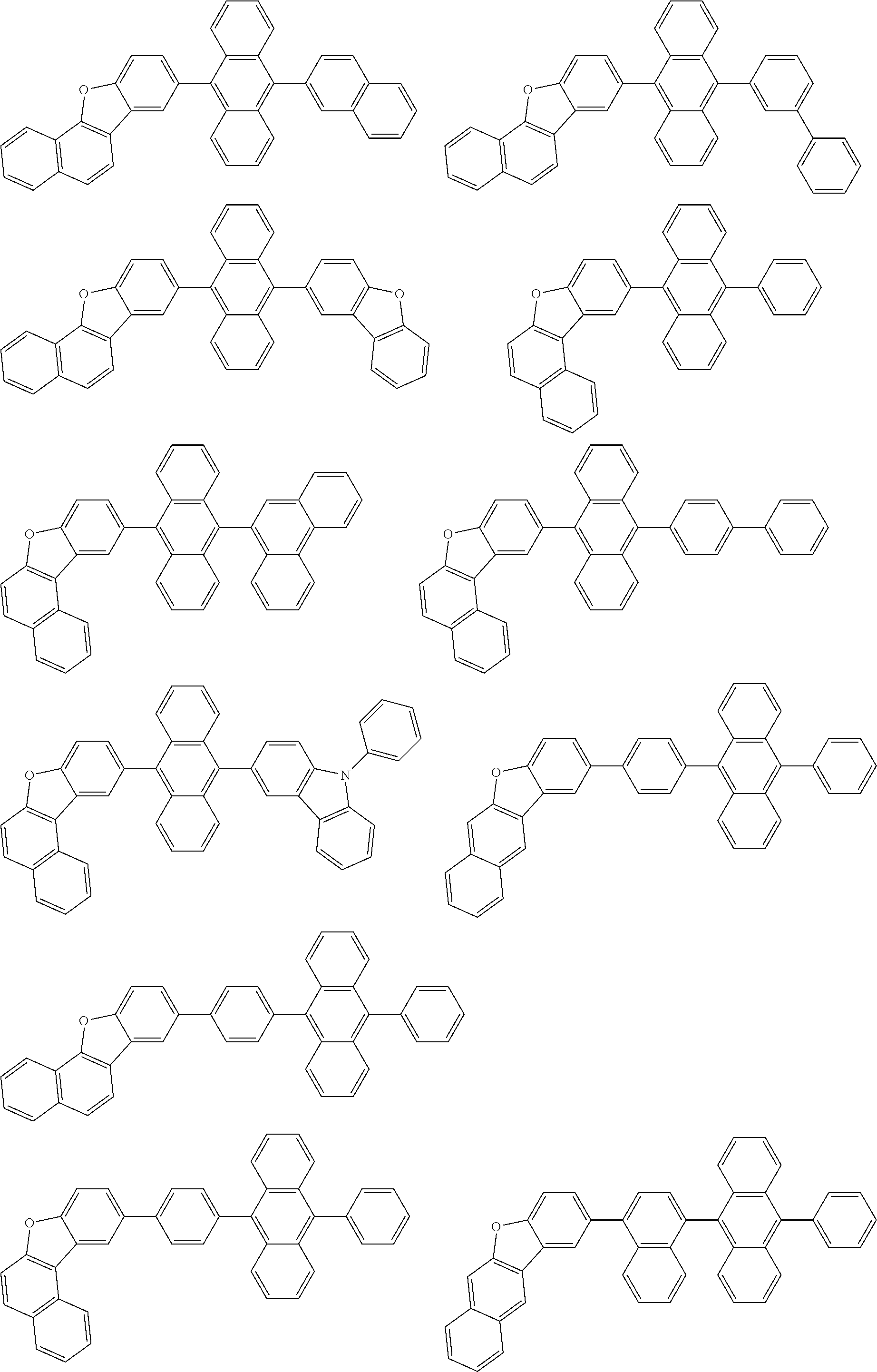

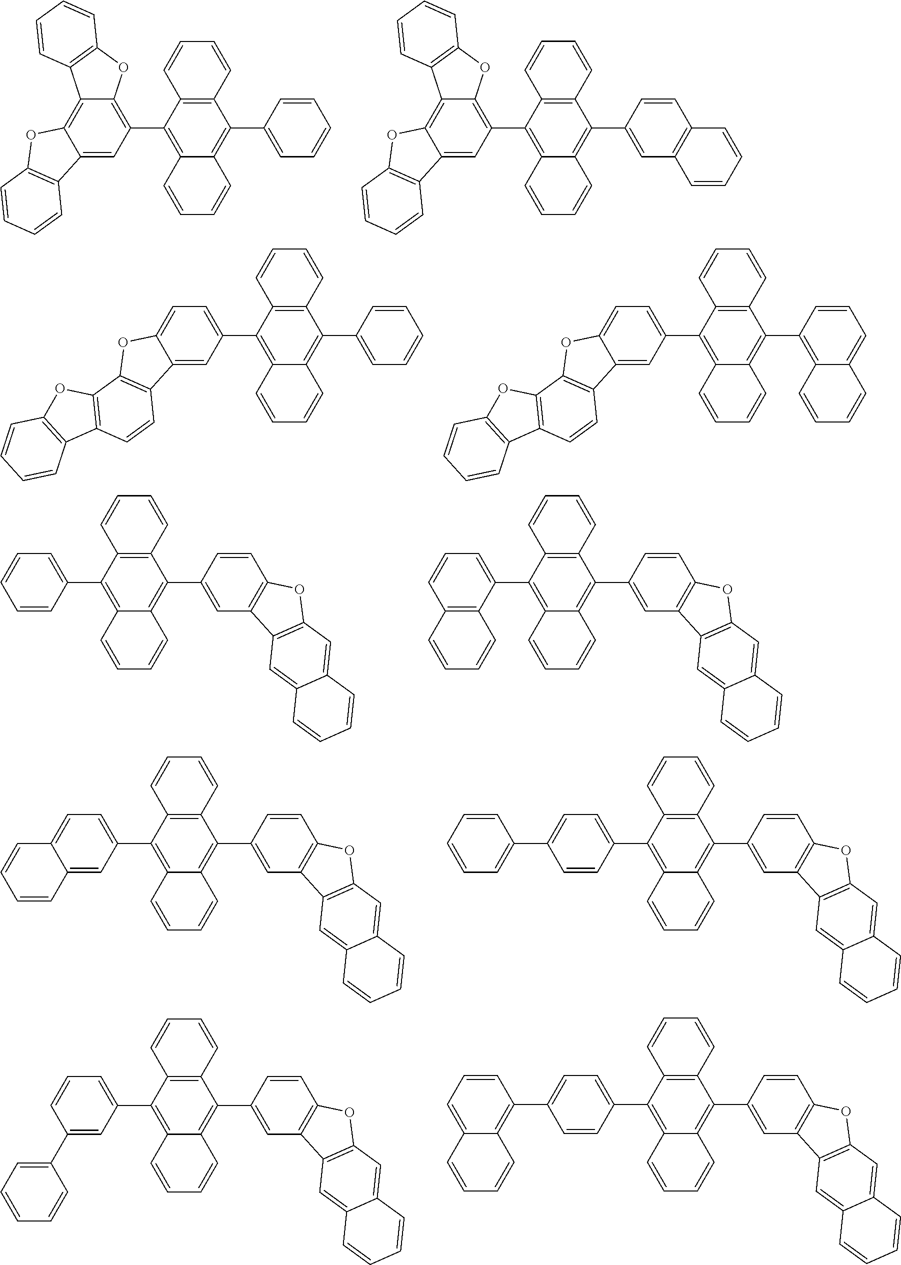

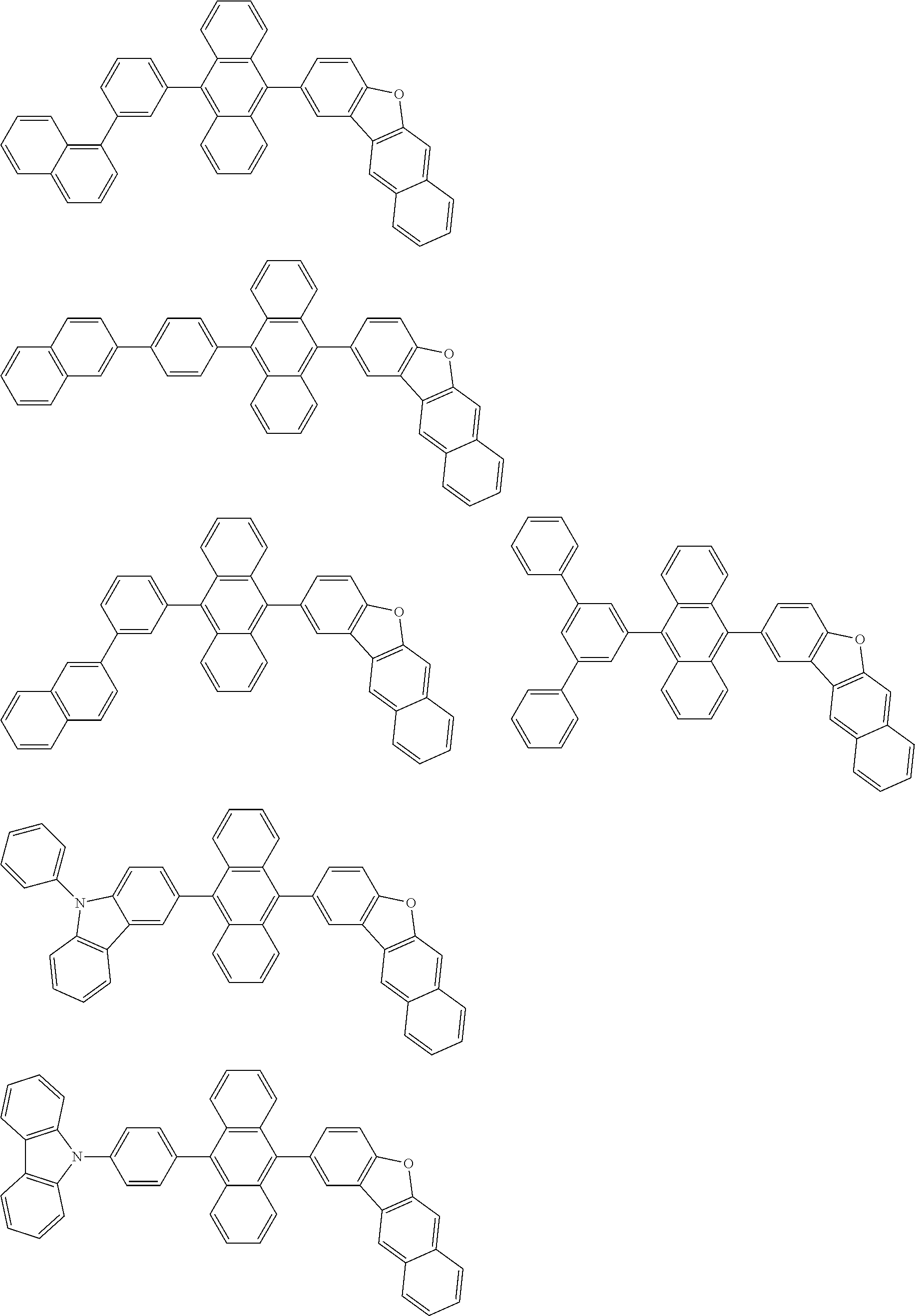

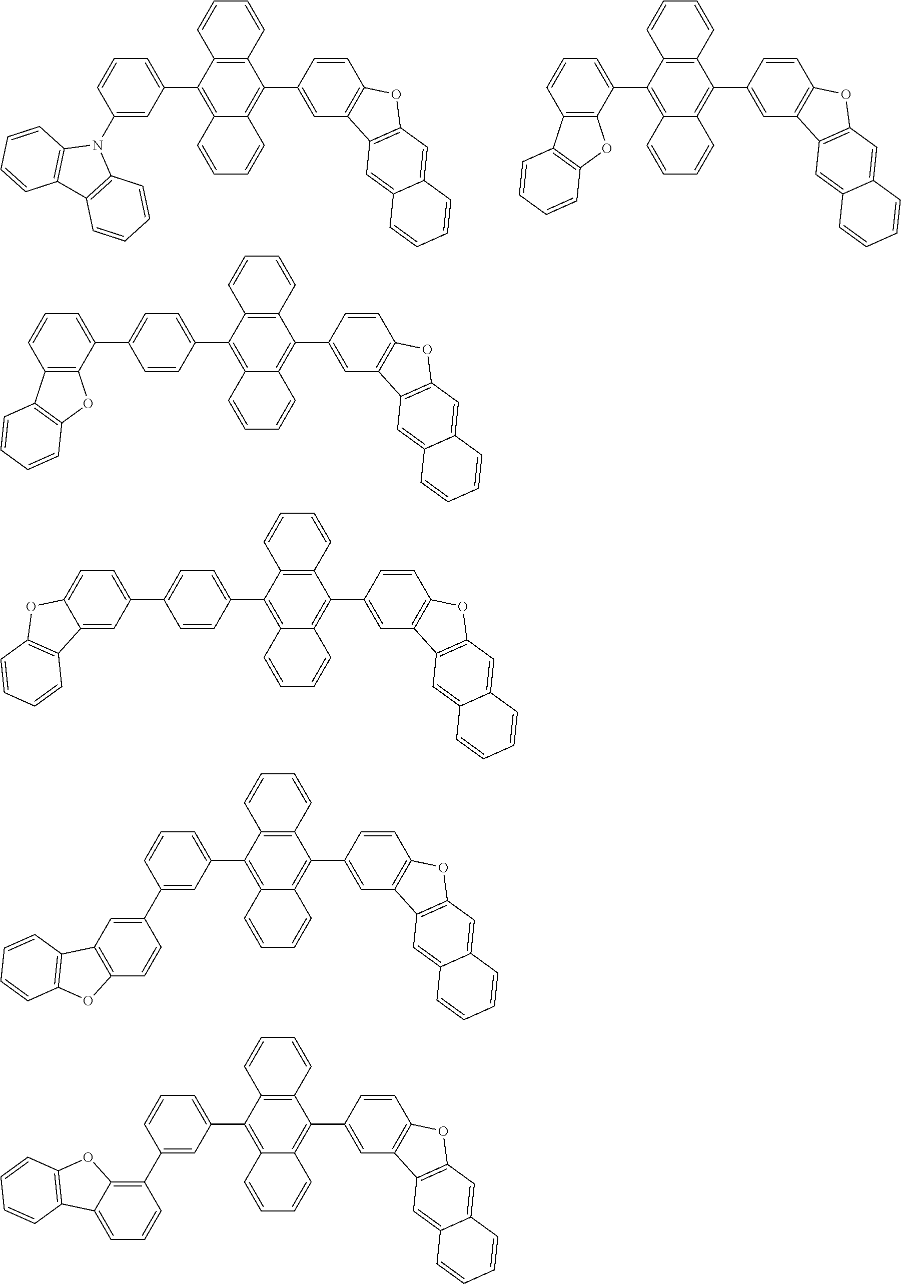 [SMILES: c1cc(-c2c3ccccc3c(-c3ccc4oc5cc6ccccc6cc5c4c3)c3ccccc23)cc(-n2c3ccccc3c3ccccc32)c1 ; c1ccc2cc3c(cc2c1)oc1ccc(-c2c4ccccc4c(-c4cccc5c4oc4ccccc45)c4ccccc24)cc13 ; c1ccc2cc3c(cc2c1)oc1ccc(-c2c4ccccc4c(-c4ccc(-c5cccc6c5oc5ccccc56)cc4)c4ccccc24)cc13 ; c1ccc2cc3c(cc2c1)oc1ccc(-c2c4ccccc4c(-c4ccc(-c5ccc6oc7ccccc7c6c5)cc4)c4ccccc24)cc13 ; c1cc(-c2ccc3oc4ccccc4c3c2)cc(-c2c3ccccc3c(-c3ccc4oc5cc6ccccc6cc5c4c3)c3ccccc23)c1 ; c1cc(-c2c3ccccc3c(-c3ccc4oc5cc6ccccc6cc5c4c3)c3ccccc23)cc(-c2cccc3c2oc2ccccc23)c1]

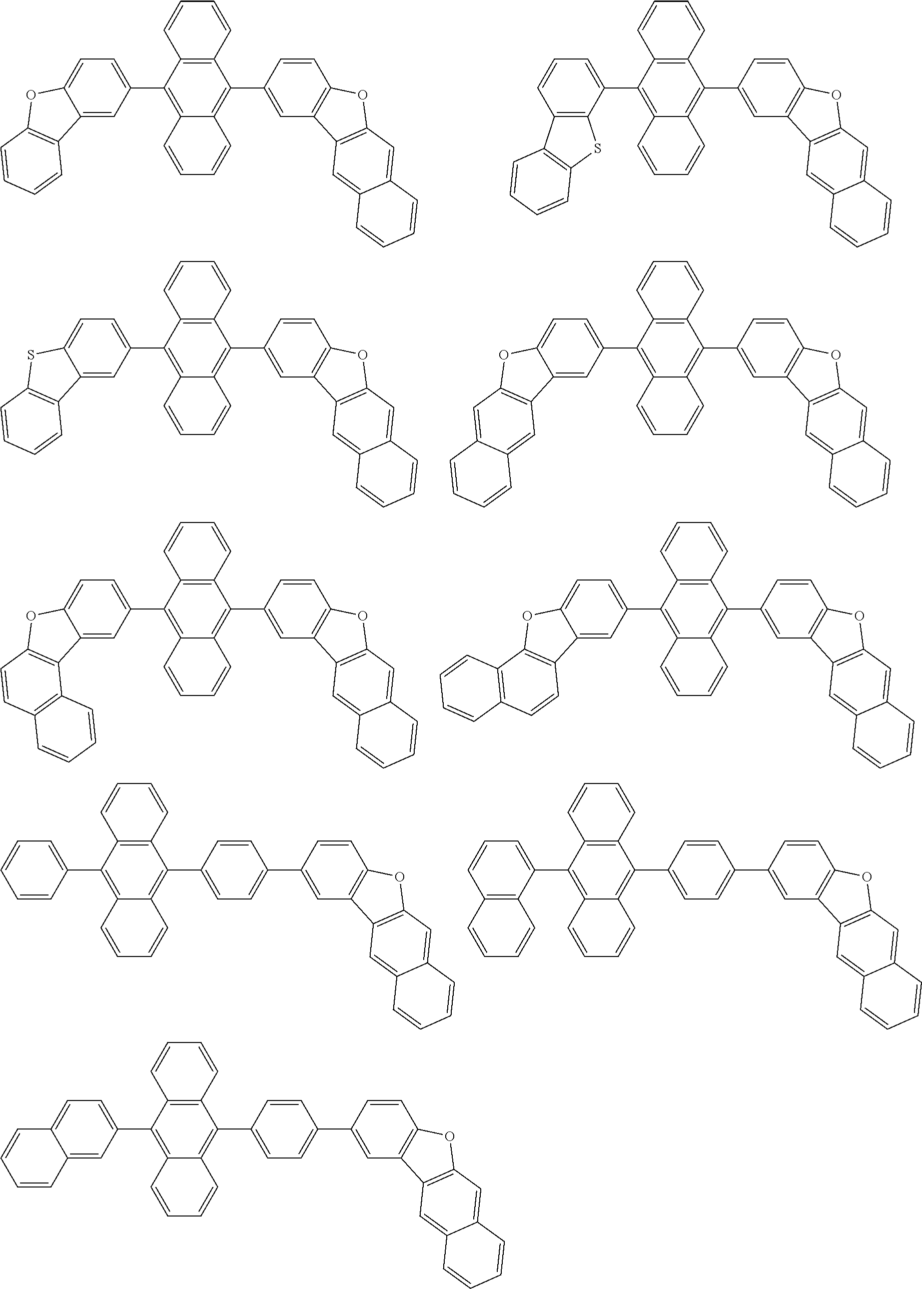

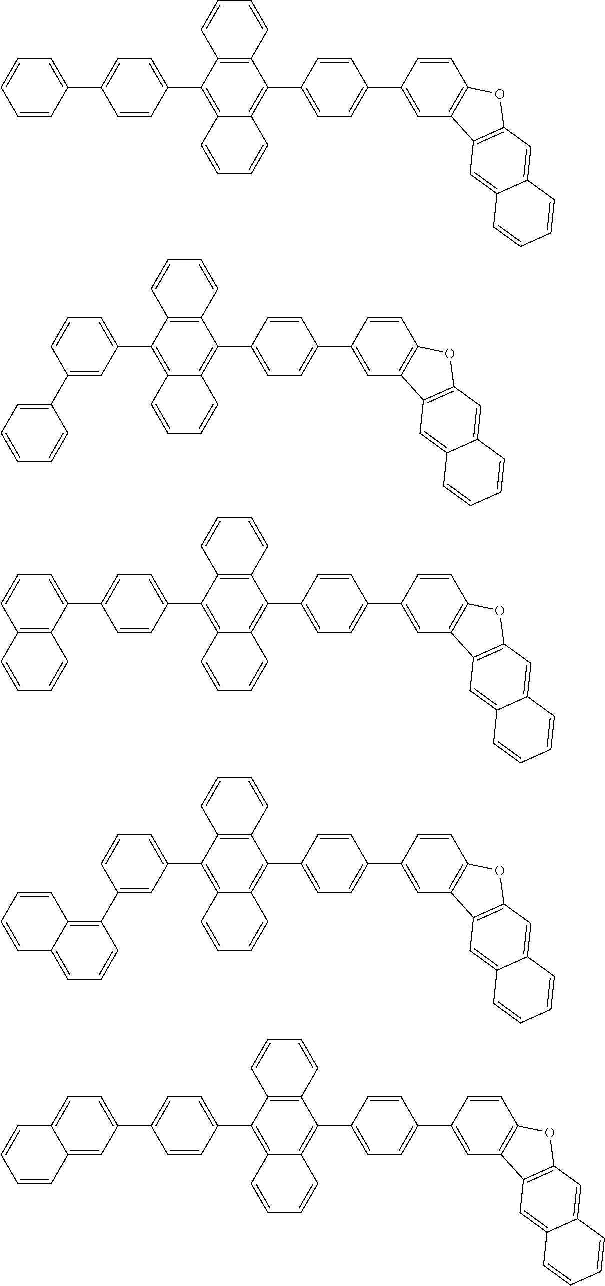 [SMILES: c1ccc(-c2ccc(-c3c4ccccc4c(-c4ccc(-c5ccc6oc7cc8ccccc8cc7c6c5)cc4)c4ccccc34)cc2)cc1 ; c1ccc(-c2cccc(-c3c4ccccc4c(-c4ccc(-c5ccc6oc7cc8ccccc8cc7c6c5)cc4)c4ccccc34)c2)cc1 ; c1ccc2cc3c(cc2c1)oc1ccc(-c2ccc(-c4c5ccccc5c(-c5ccc(-c6cccc7ccccc67)cc5)c5ccccc45)cc2)cc13 ; c1cc(-c2cccc3ccccc23)cc(-c2c3ccccc3c(-c3ccc(-c4ccc5oc6cc7ccccc7cc6c5c4)cc3)c3ccccc23)c1 ; c1ccc2cc(-c3ccc(-c4c5ccccc5c(-c5ccc(-c6ccc7oc8cc9ccccc9cc8c7c6)cc5)c5ccccc45)cc3)ccc2c1]

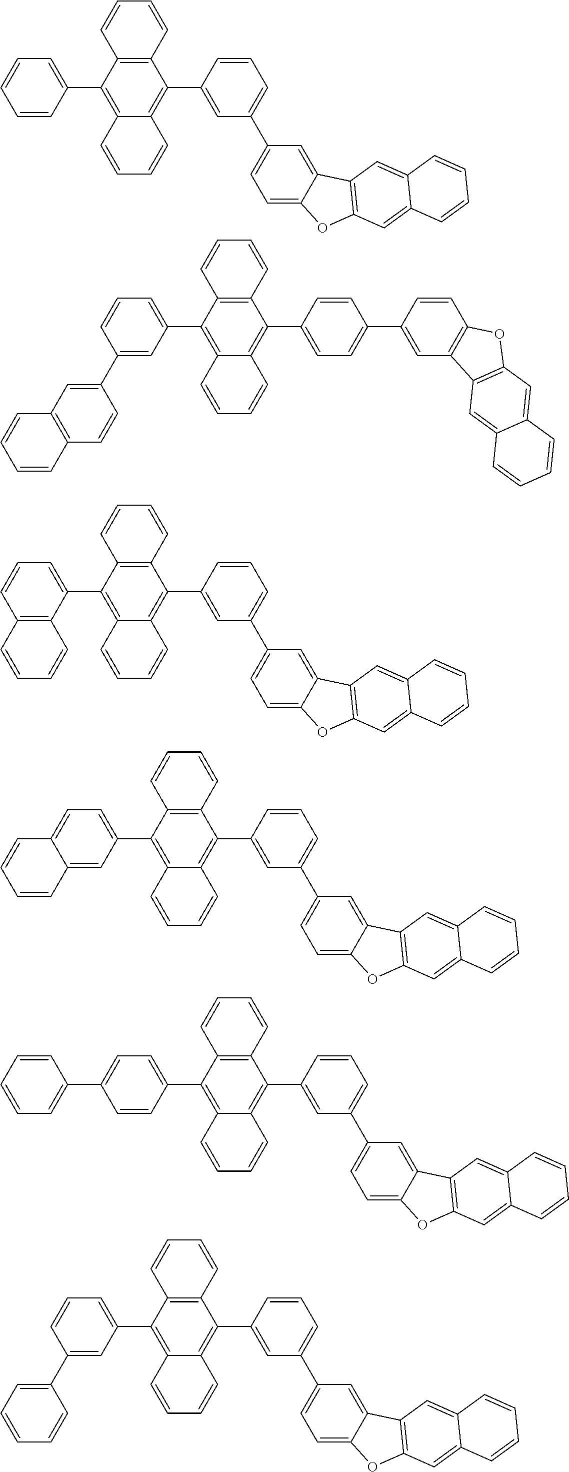

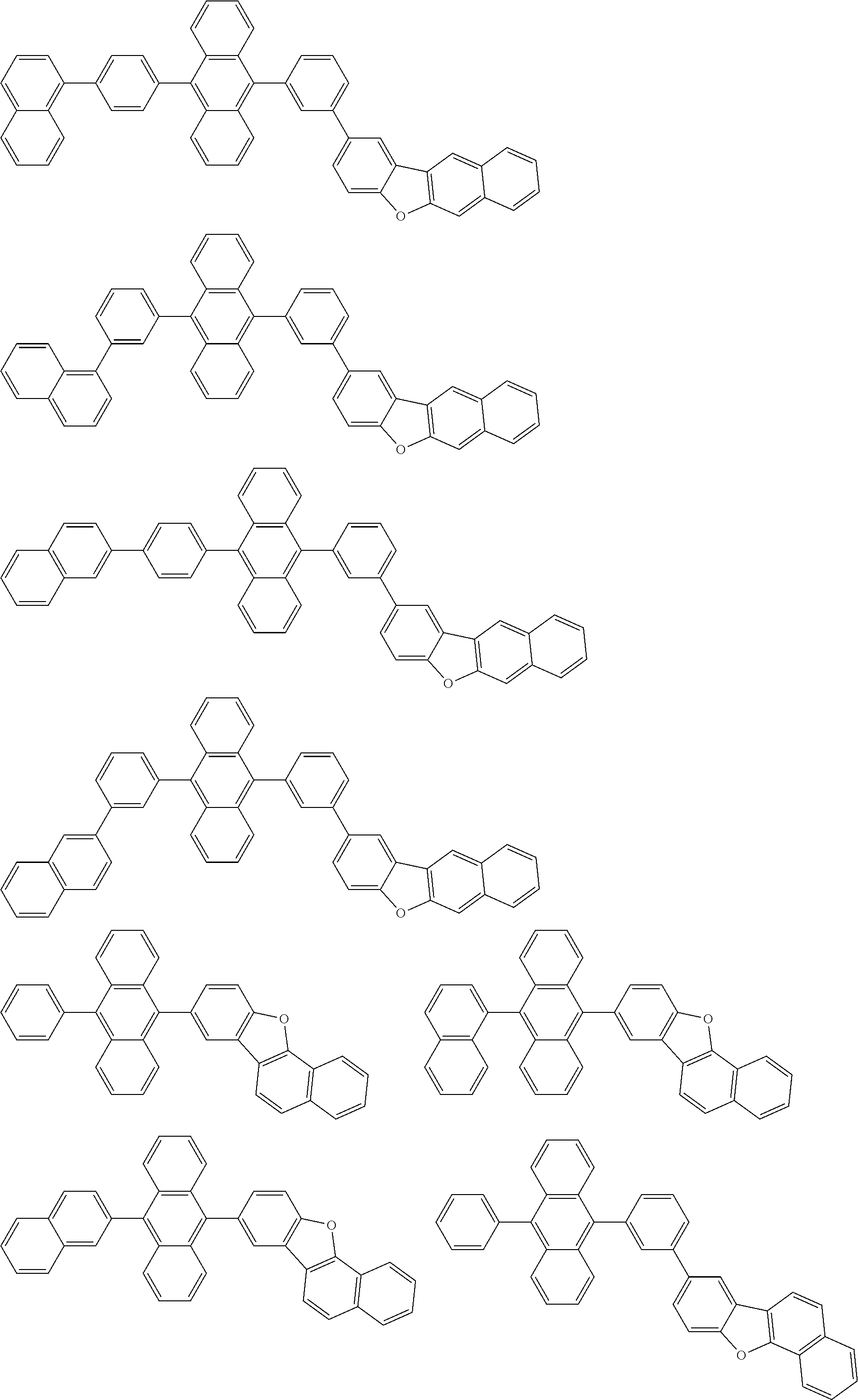

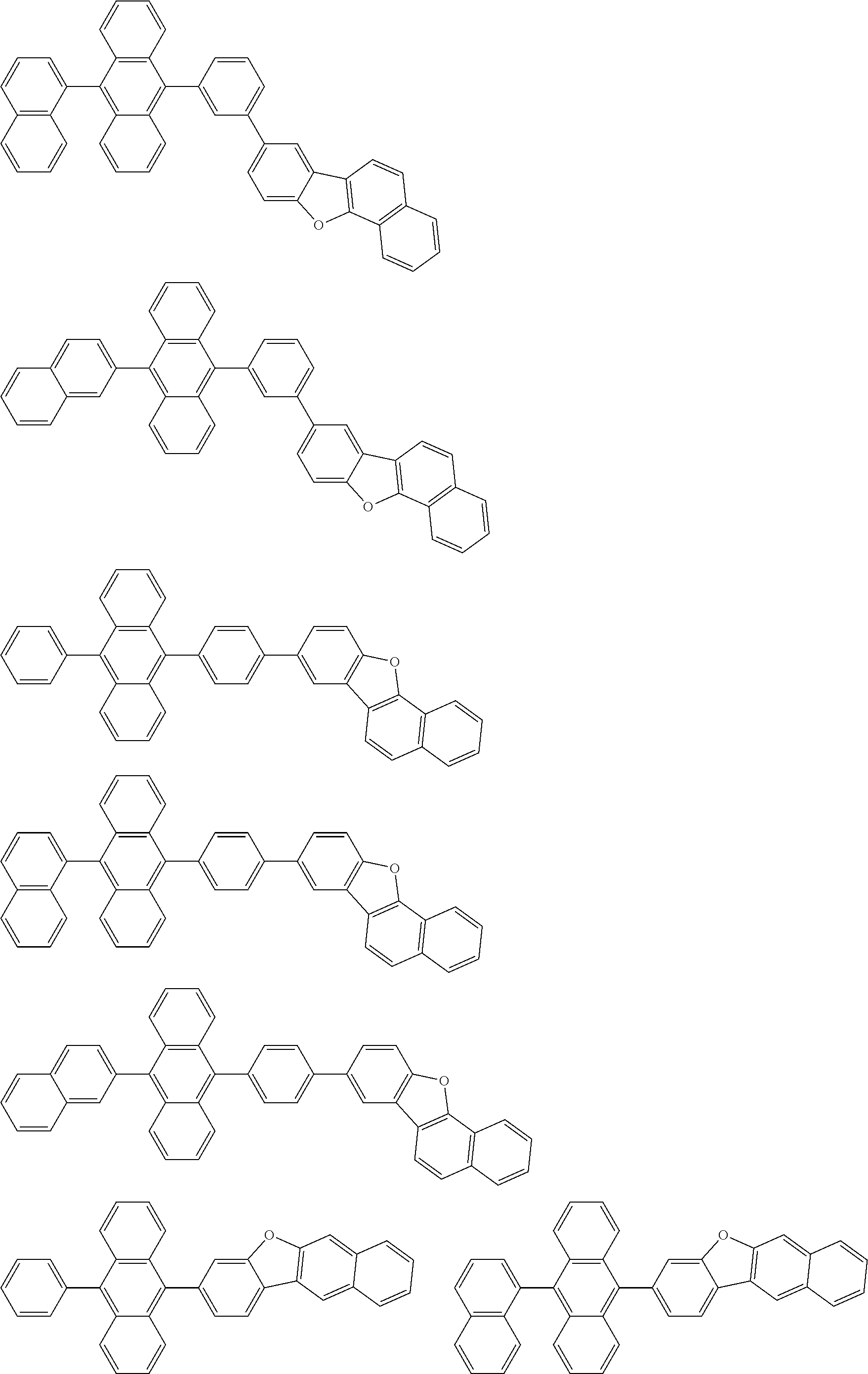

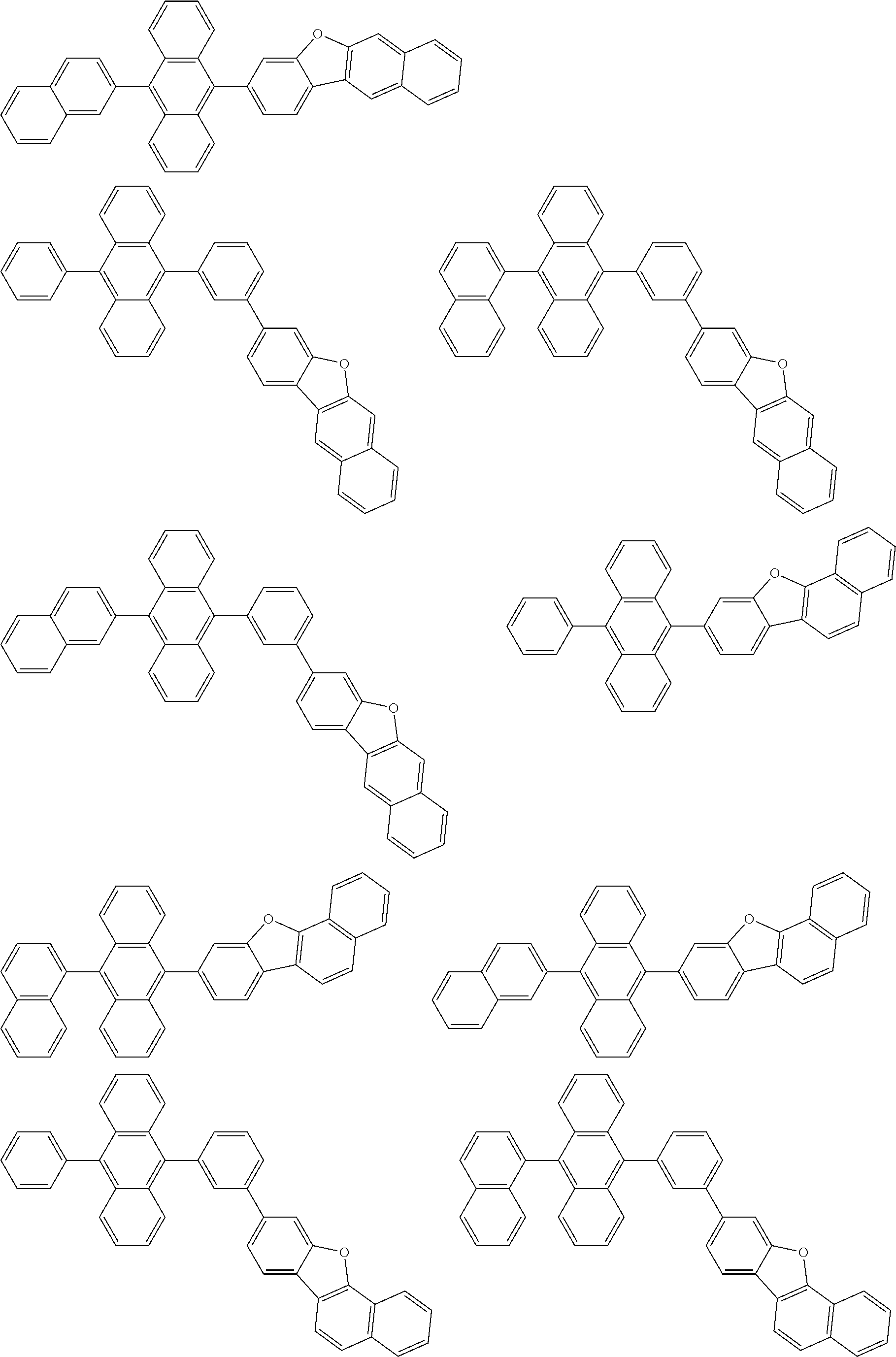

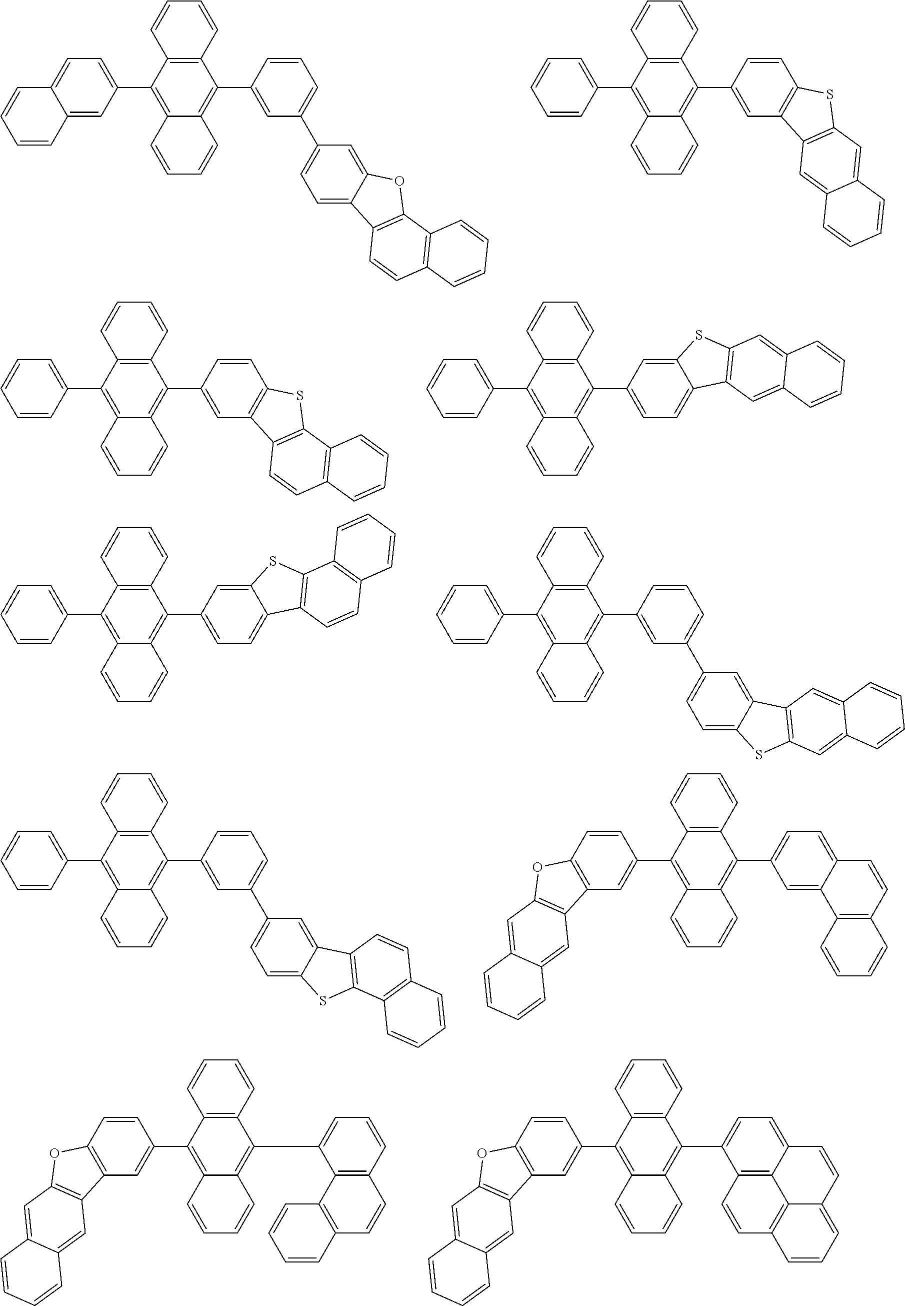

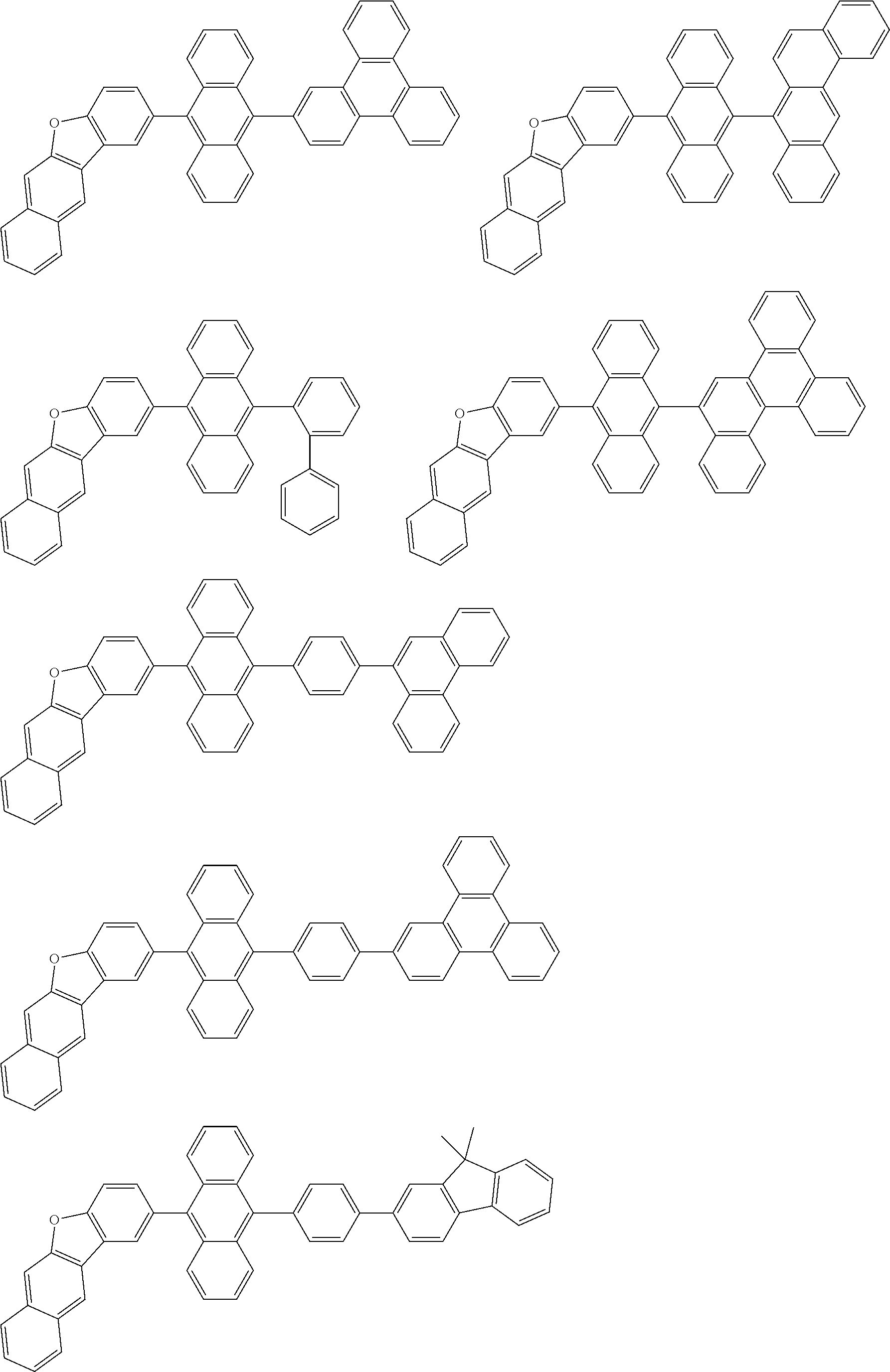

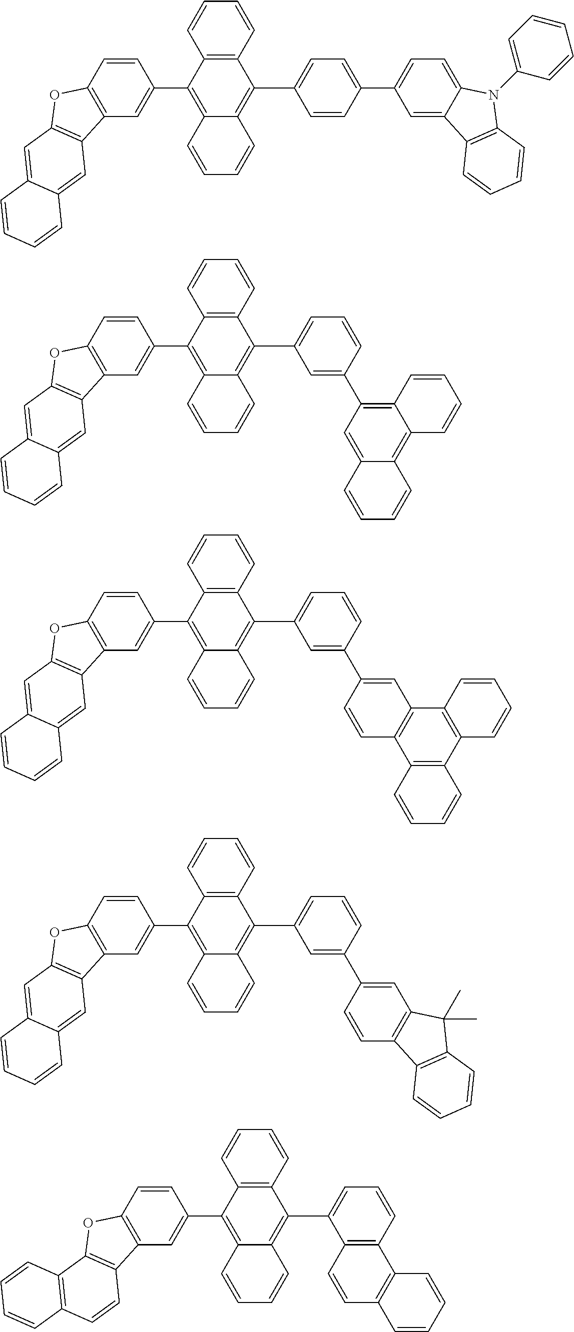 [SMILES: c1ccc(-n2c3ccccc3c3cc(-c4ccc(-c5c6ccccc6c(-c6ccc7oc8cc9ccccc9cc8c7c6)c6ccccc56)cc4)ccc32)cc1 ; c1cc(-c2c3ccccc3c(-c3ccc4oc5cc6ccccc6cc5c4c3)c3ccccc23)cc(-c2cc3ccccc3c3ccccc23)c1 ; c1cc(-c2ccc3c4ccccc4c4ccccc4c3c2)cc(-c2c3ccccc3c(-c3ccc4oc5cc6ccccc6cc5c4c3)c3ccccc23)c1 ; CC1(C)c2ccccc2-c2ccc(-c3cccc(-c4c5ccccc5c(-c5ccc6oc7cc8ccccc8cc7c6c5)c5ccccc45)c3)cc21 ; c1ccc2c(c1)ccc1c(-c3c4ccccc4c(-c4ccc5oc6c7ccccc7ccc6c5c4)c4ccccc34)cccc12]

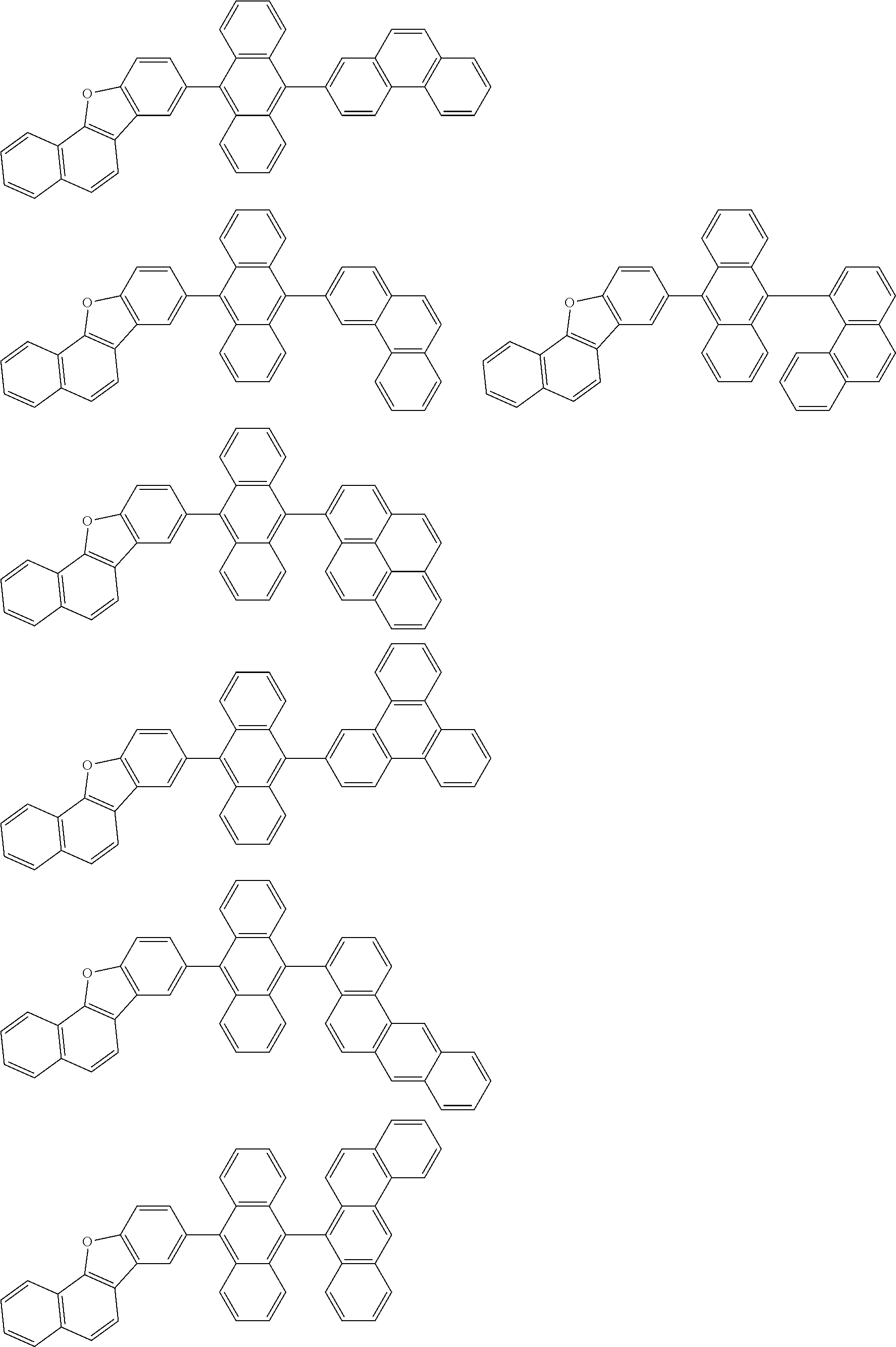

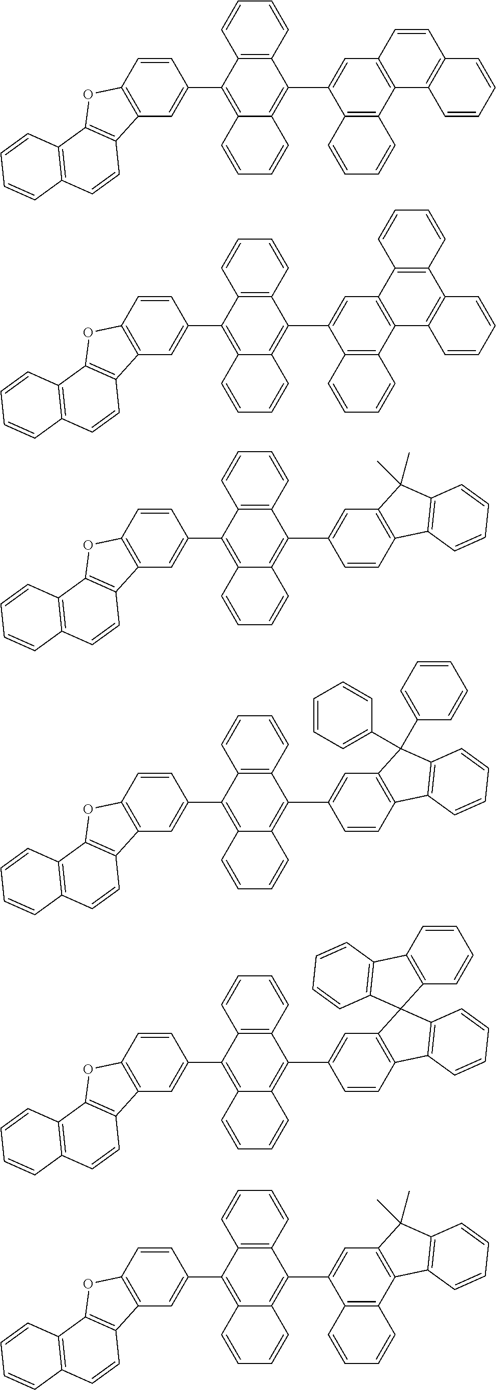

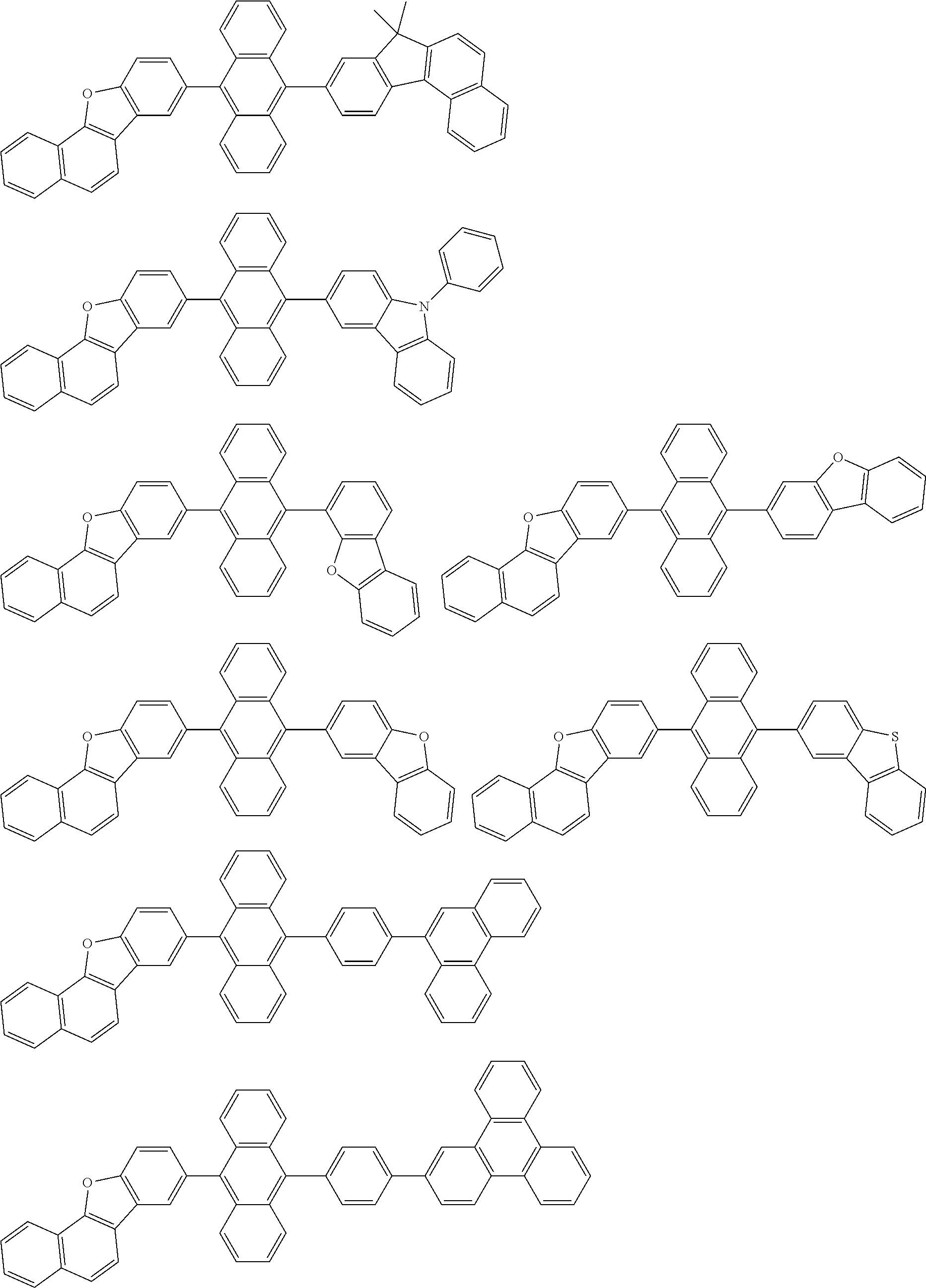

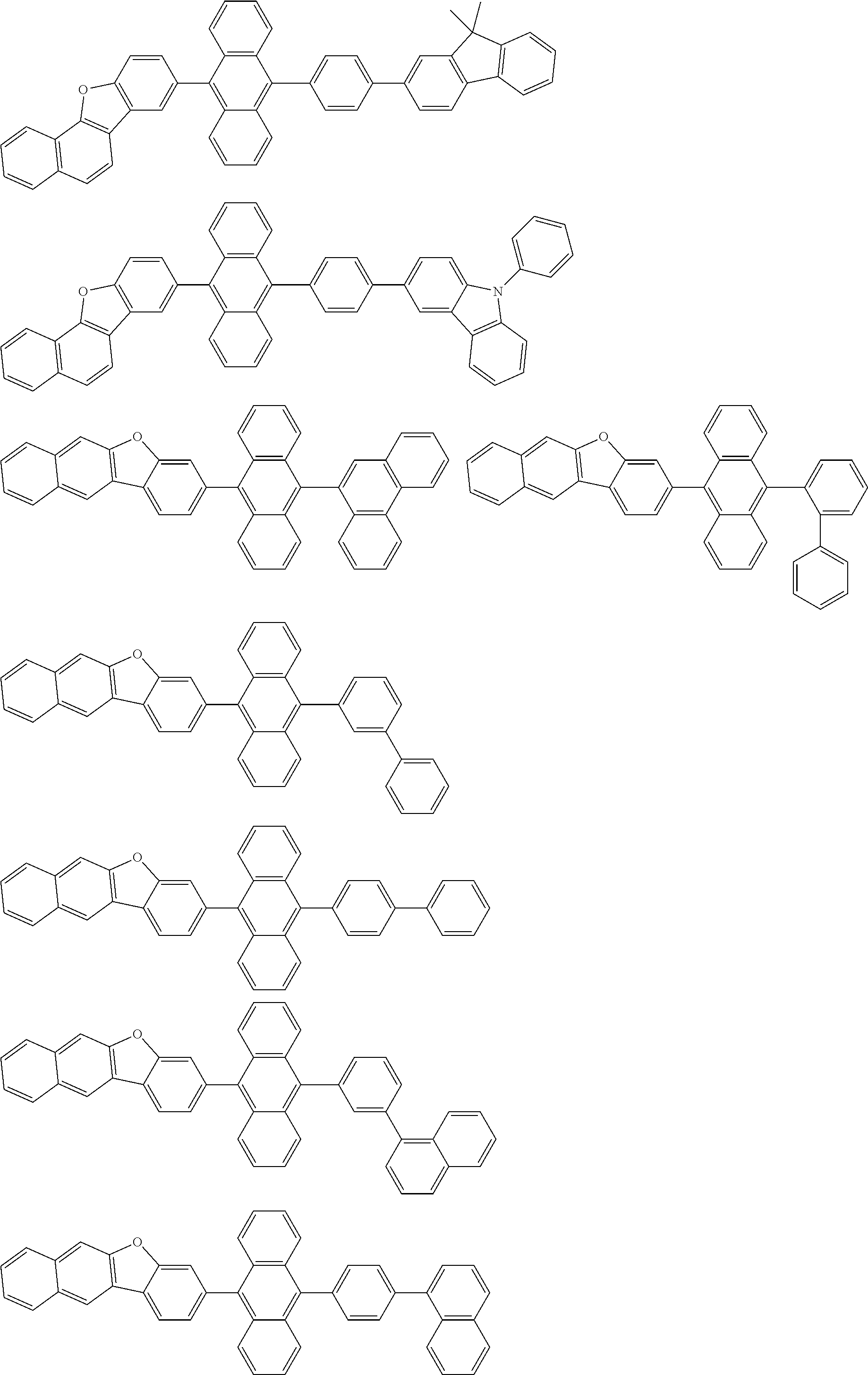

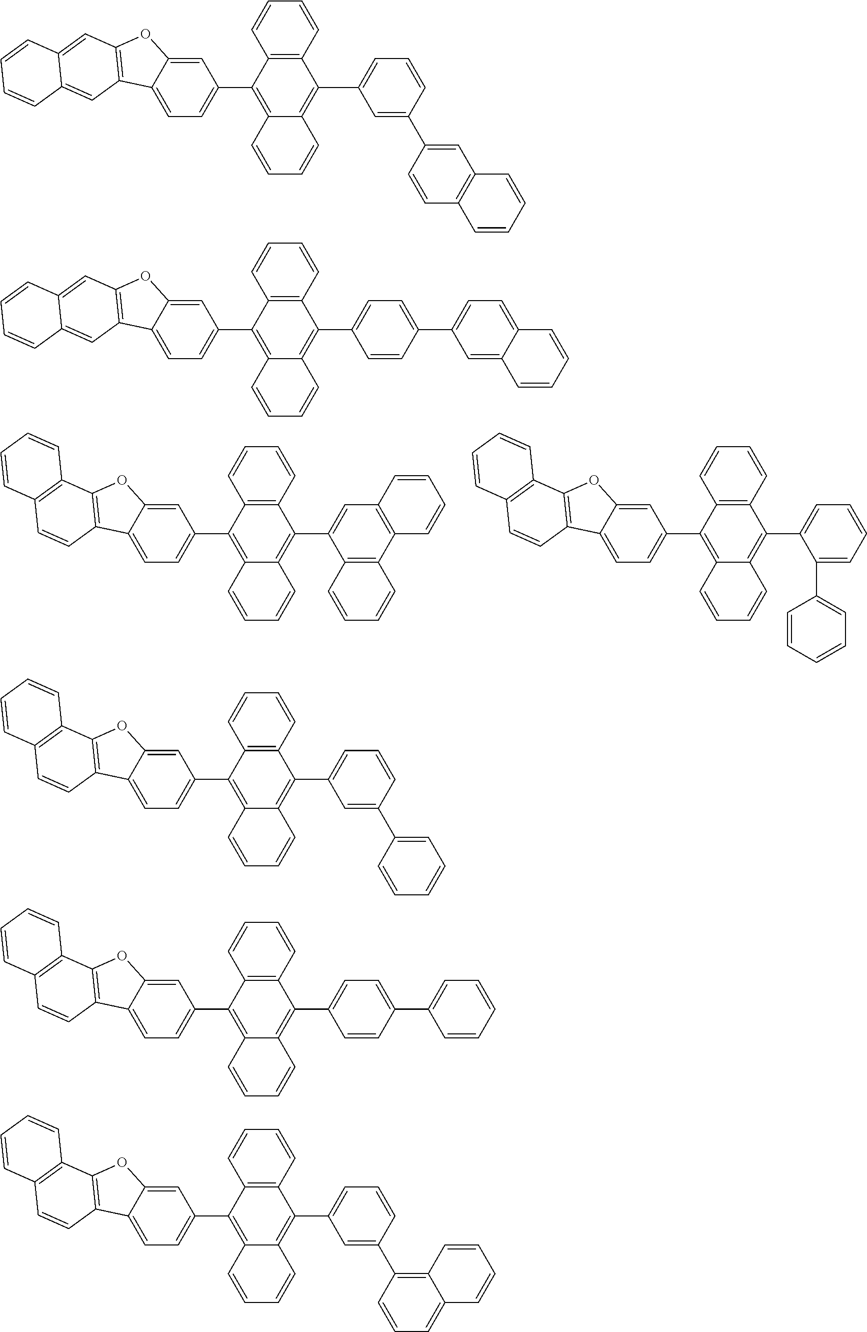

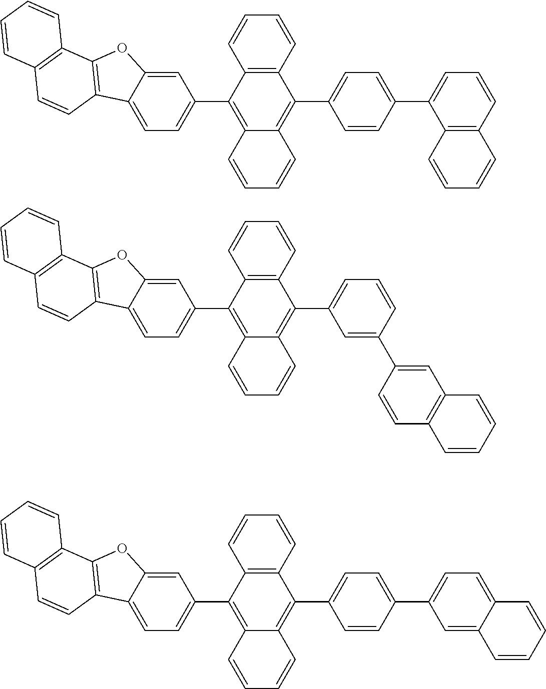

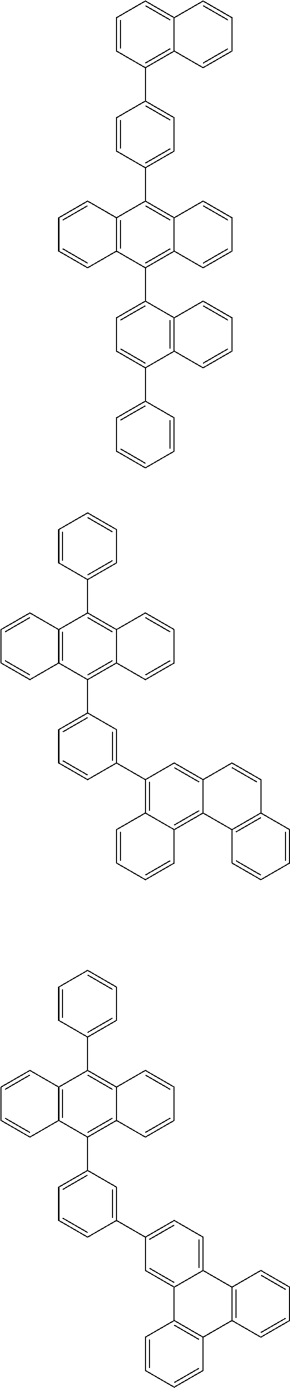

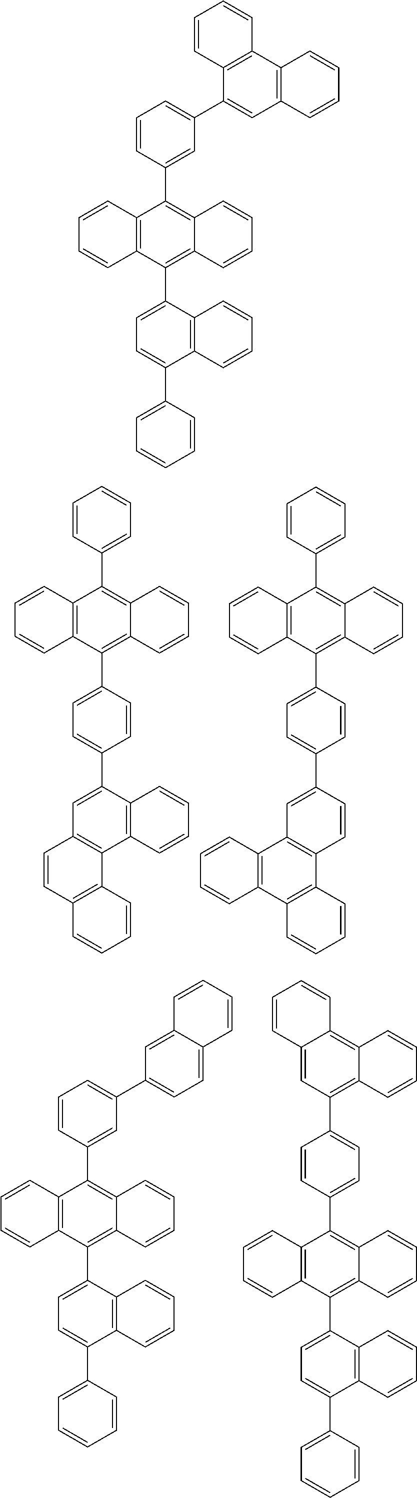

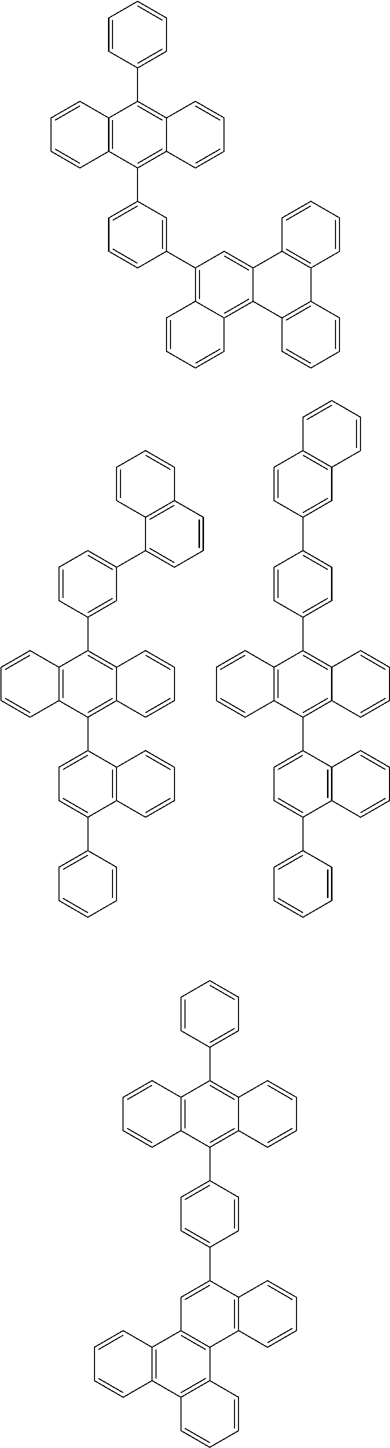

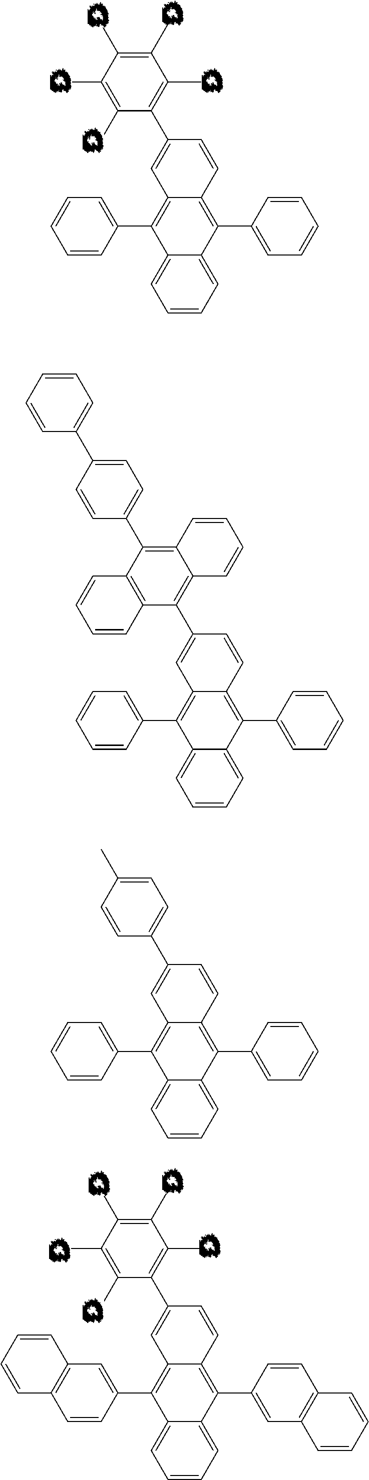

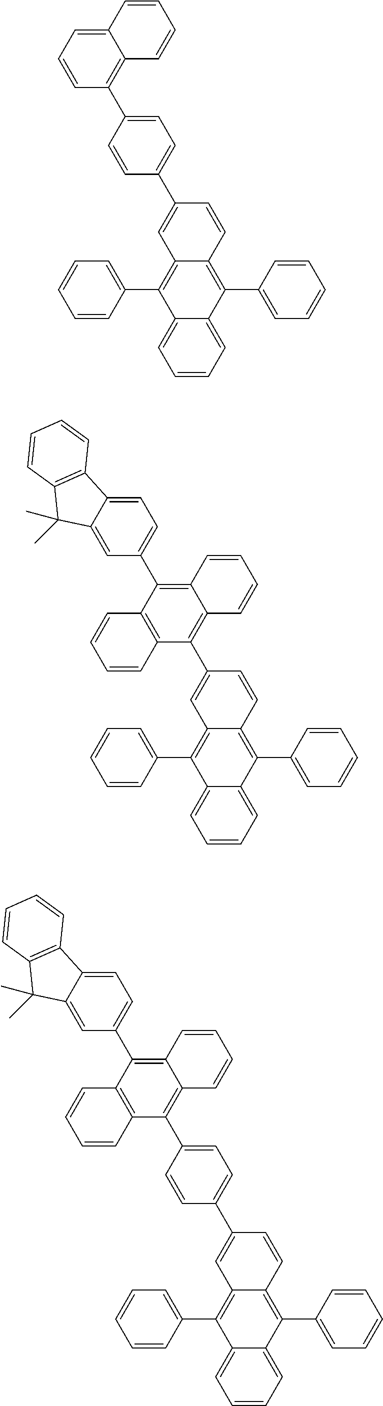

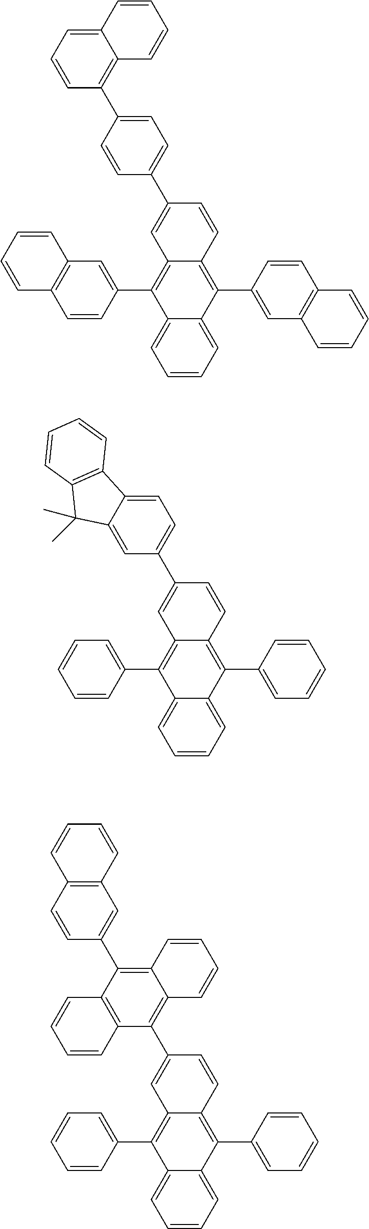

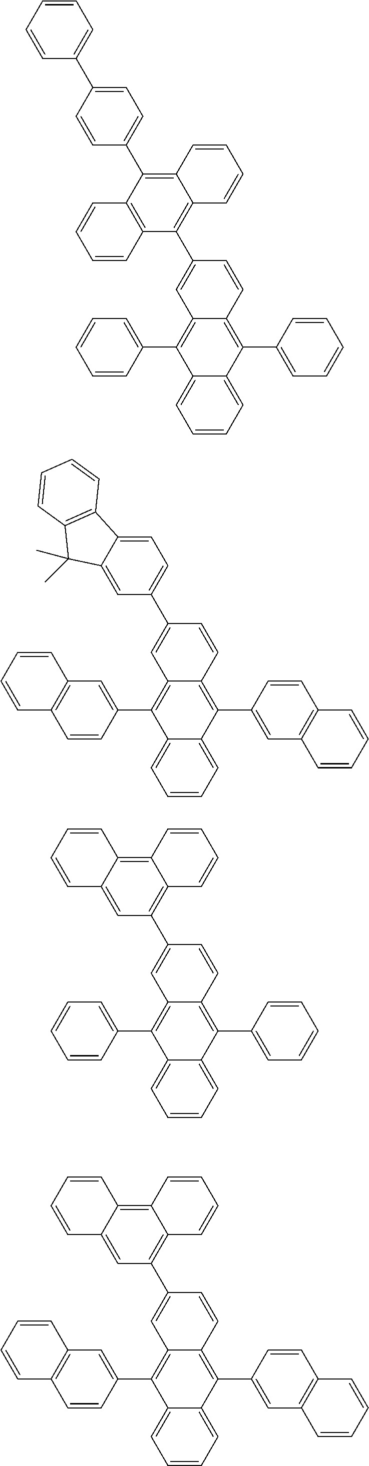

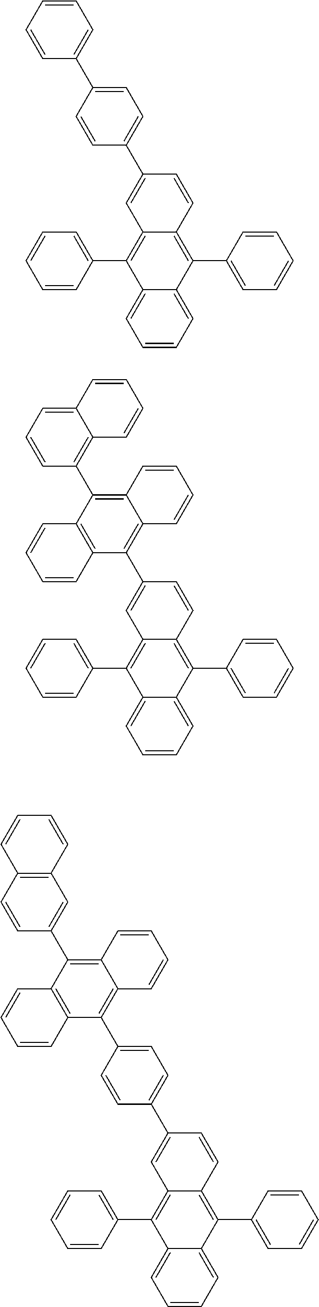

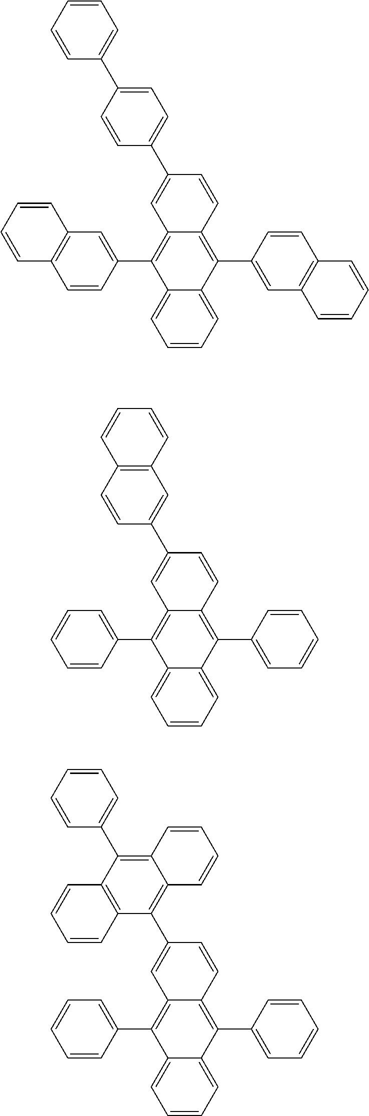

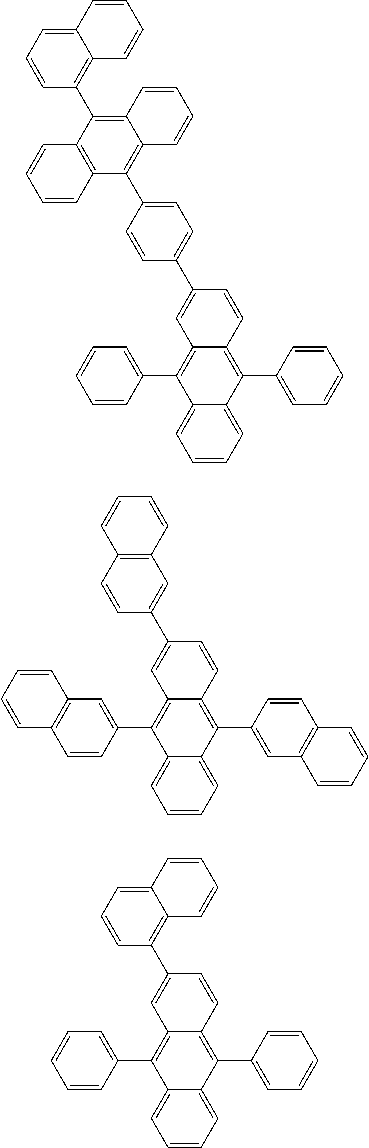

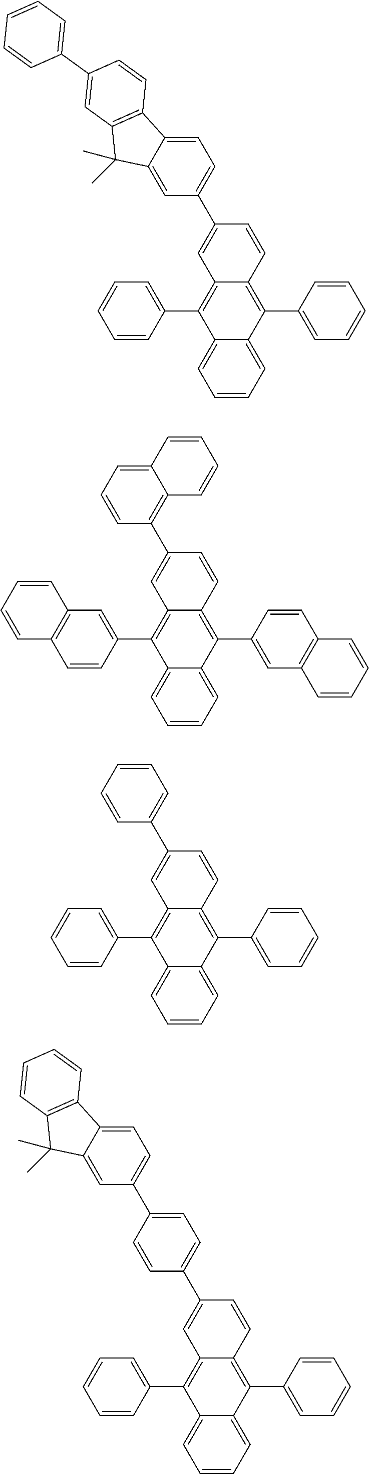

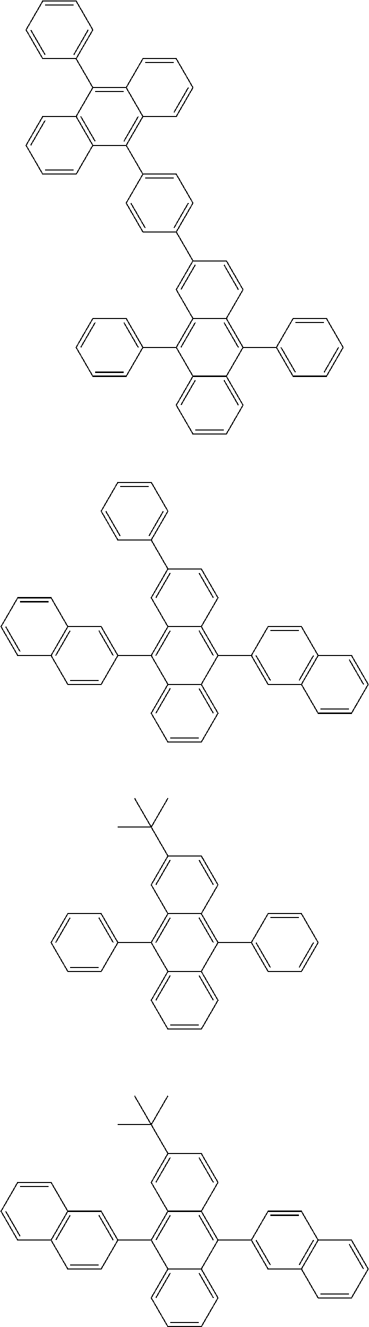

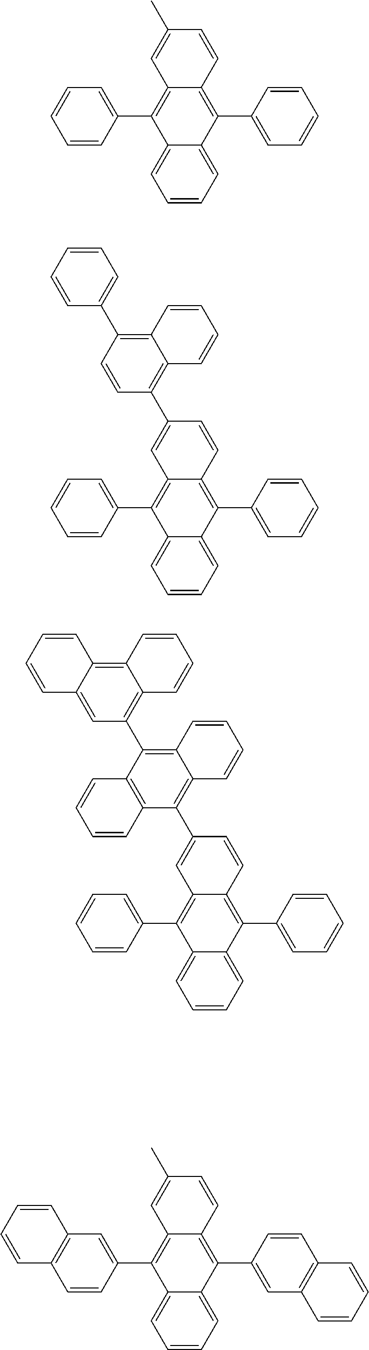

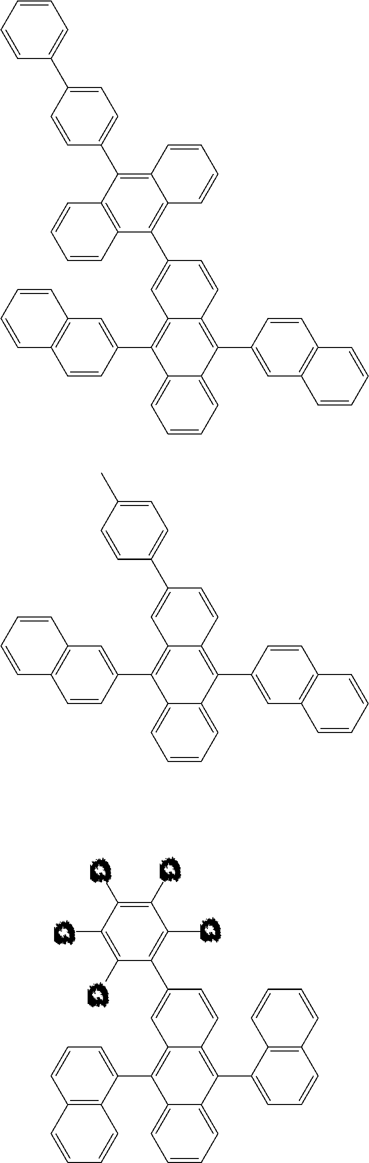

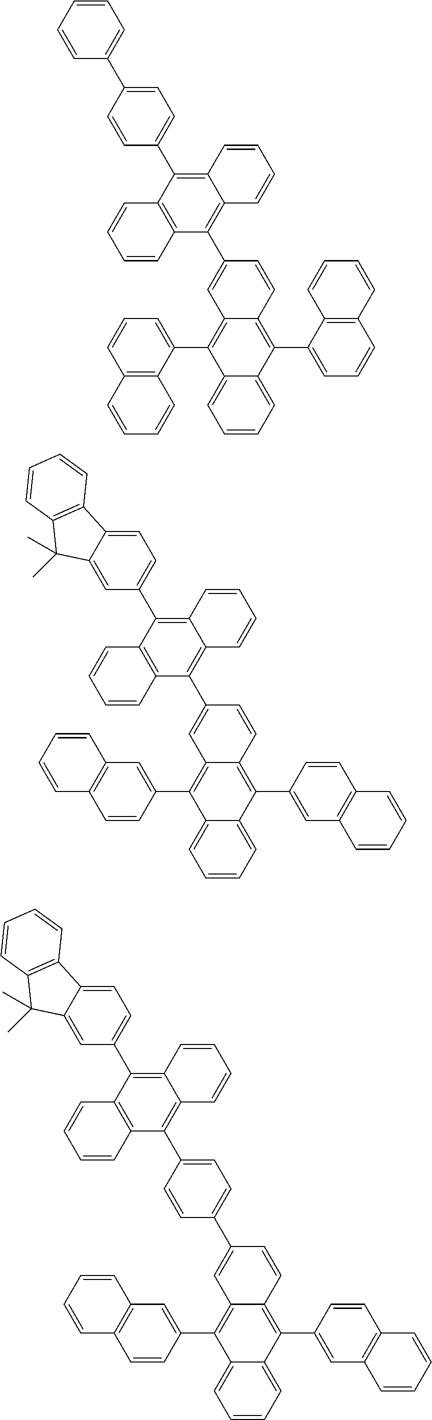

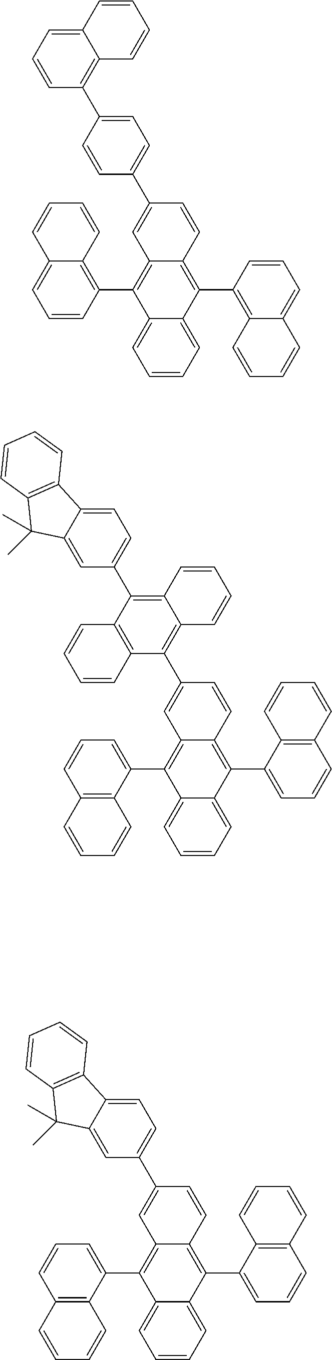

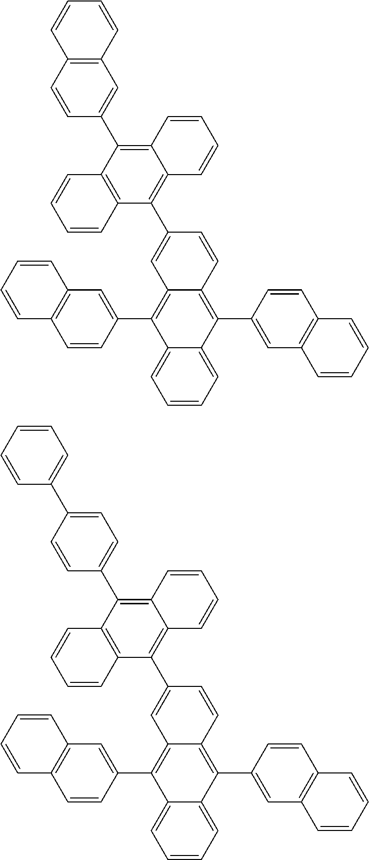

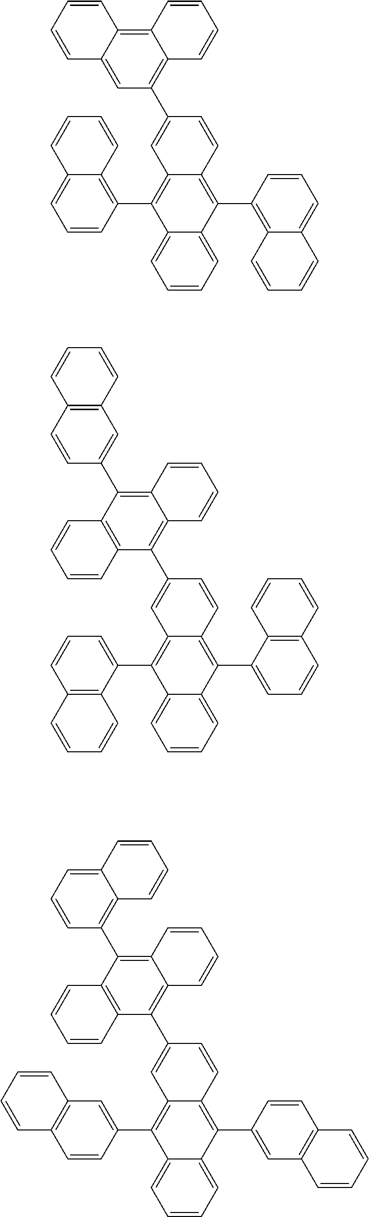

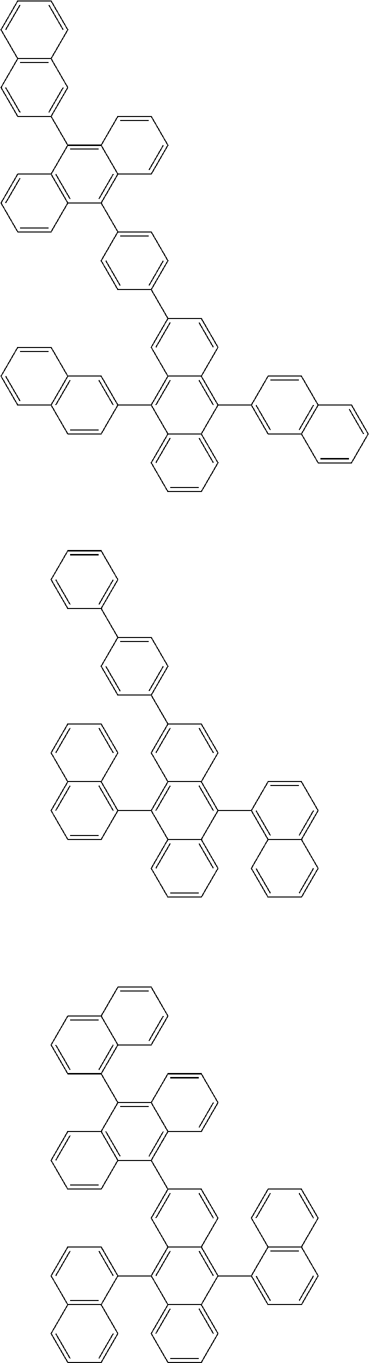

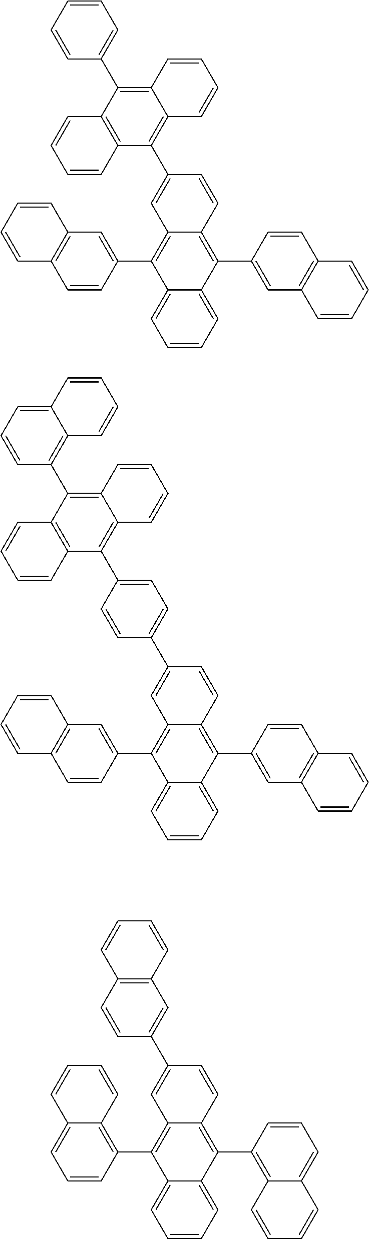

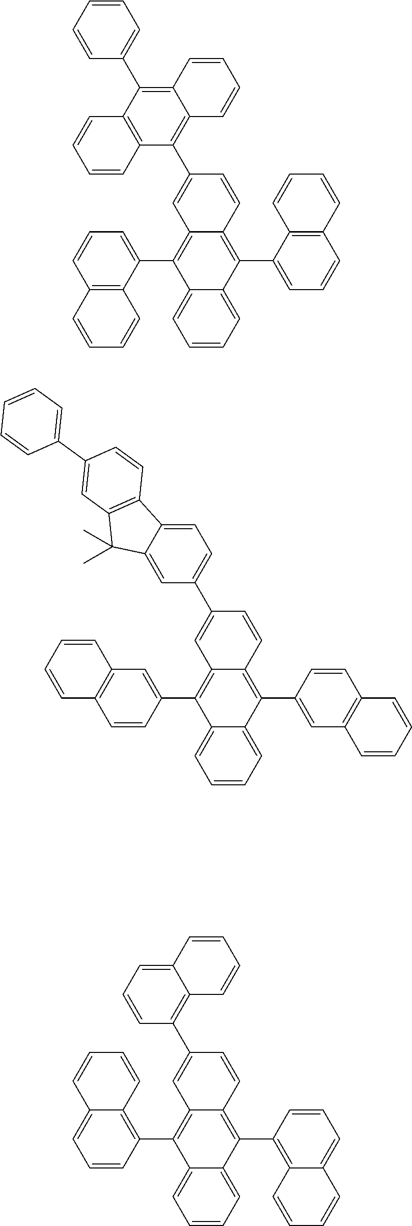

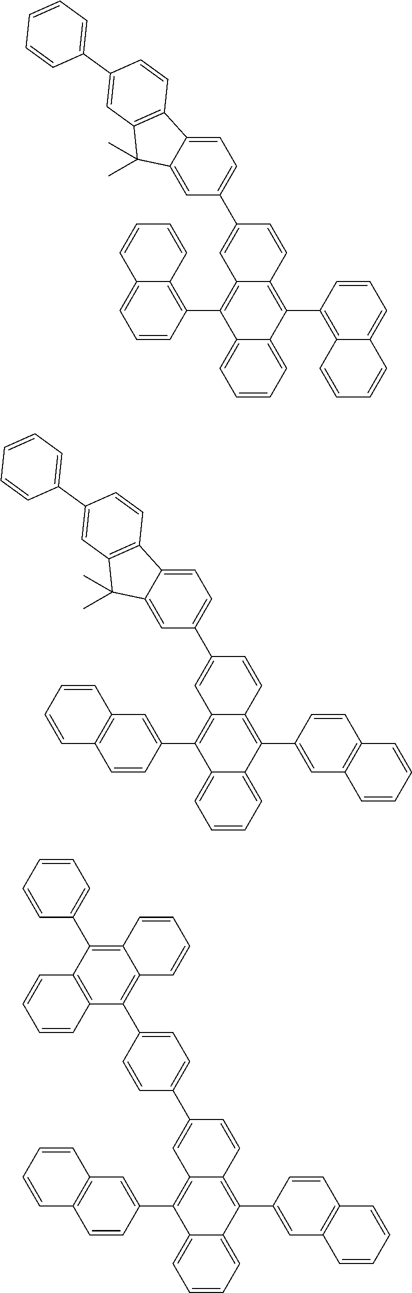

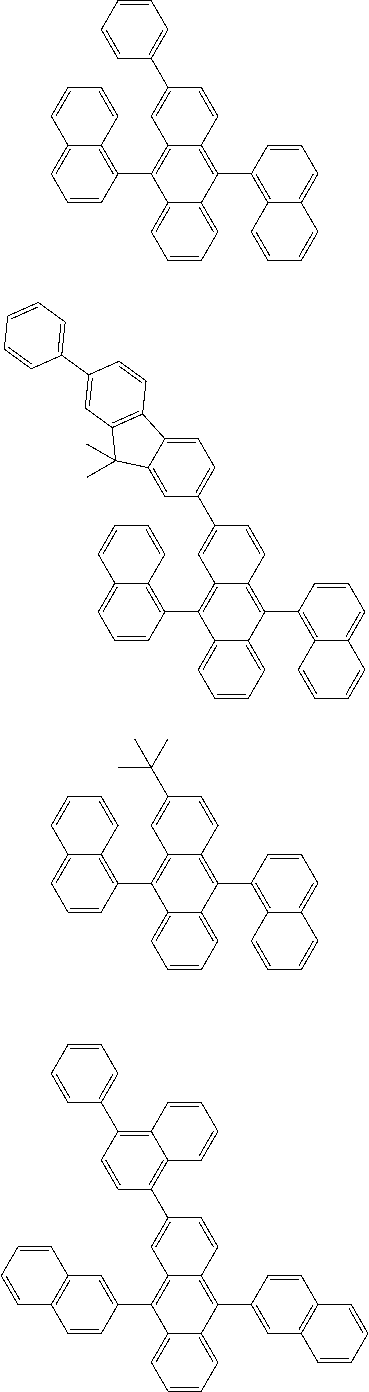

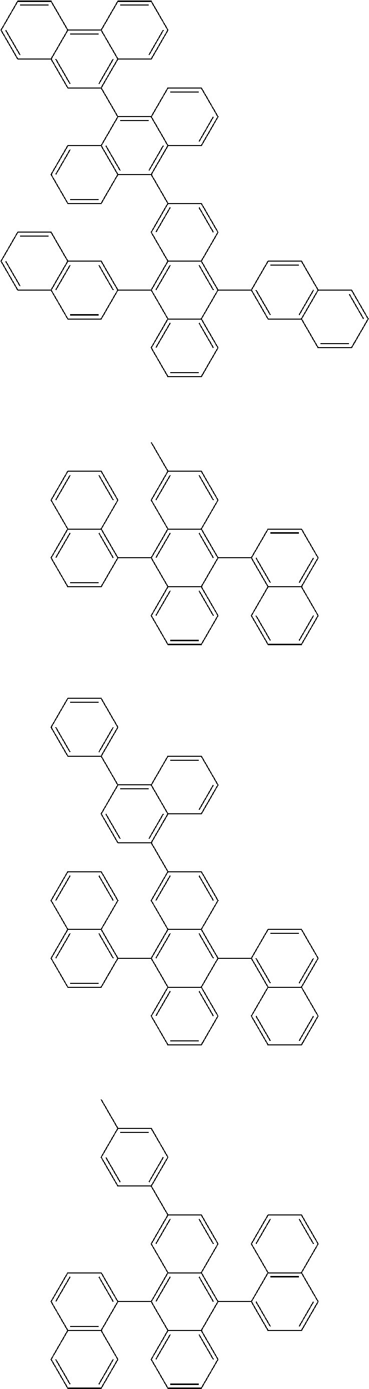

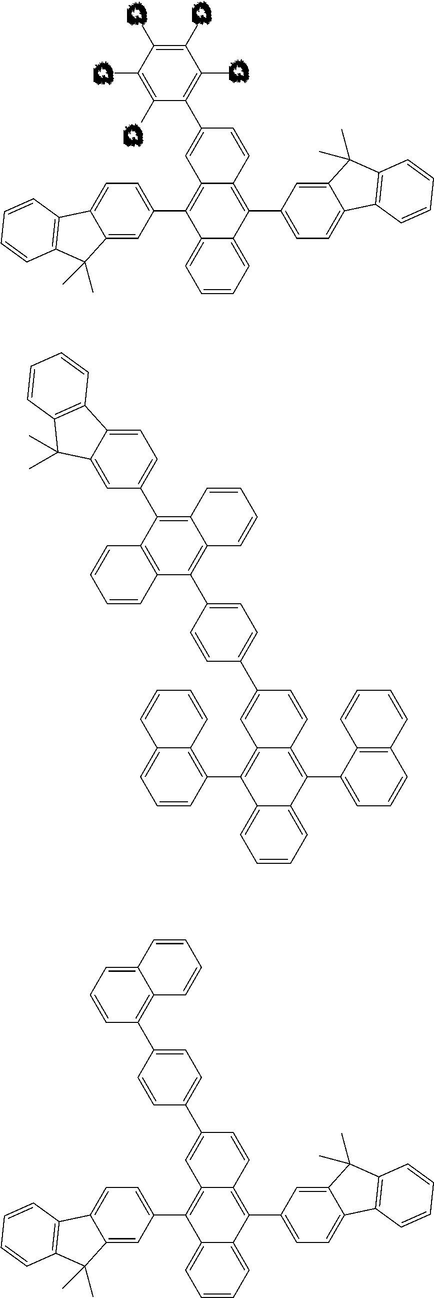

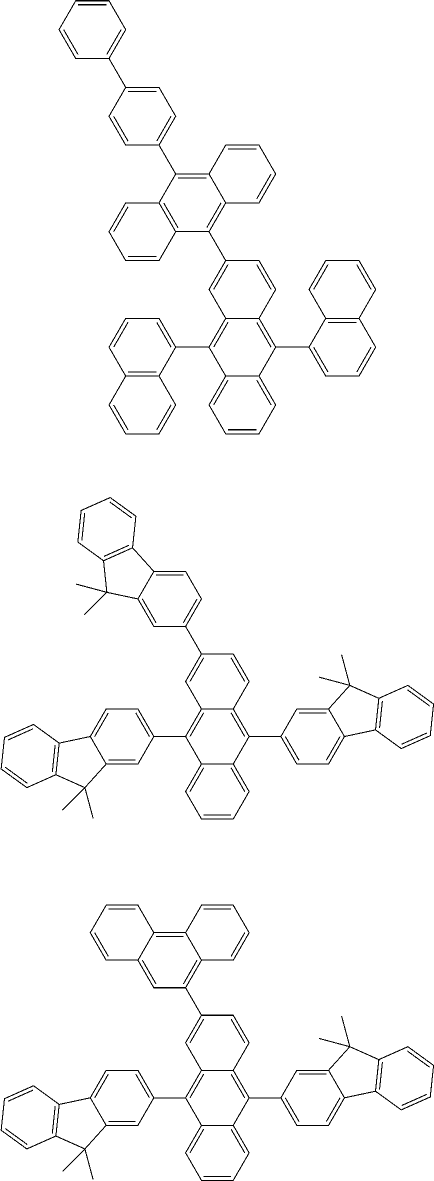

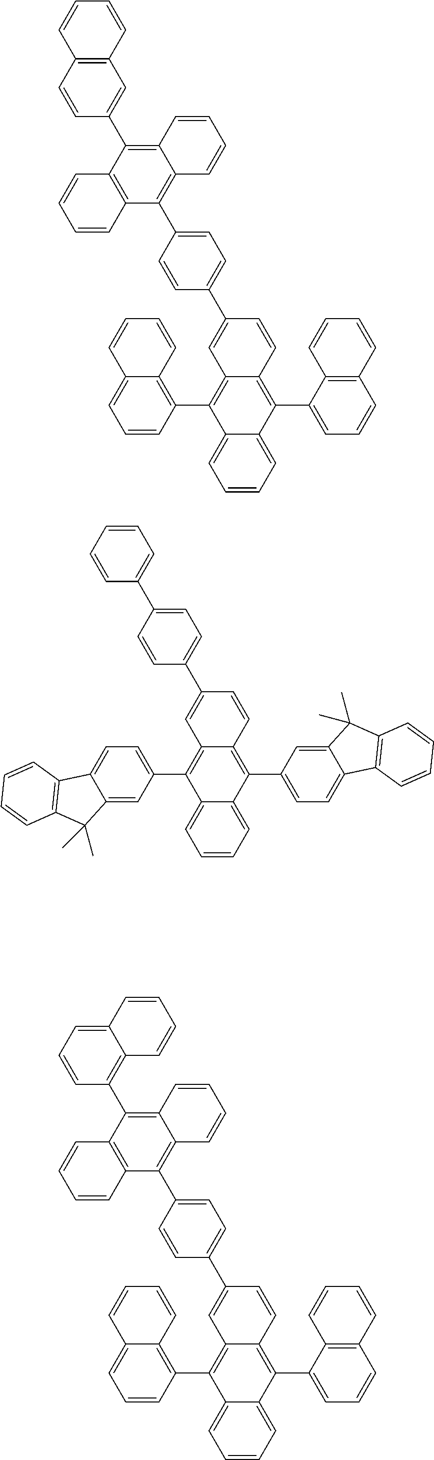

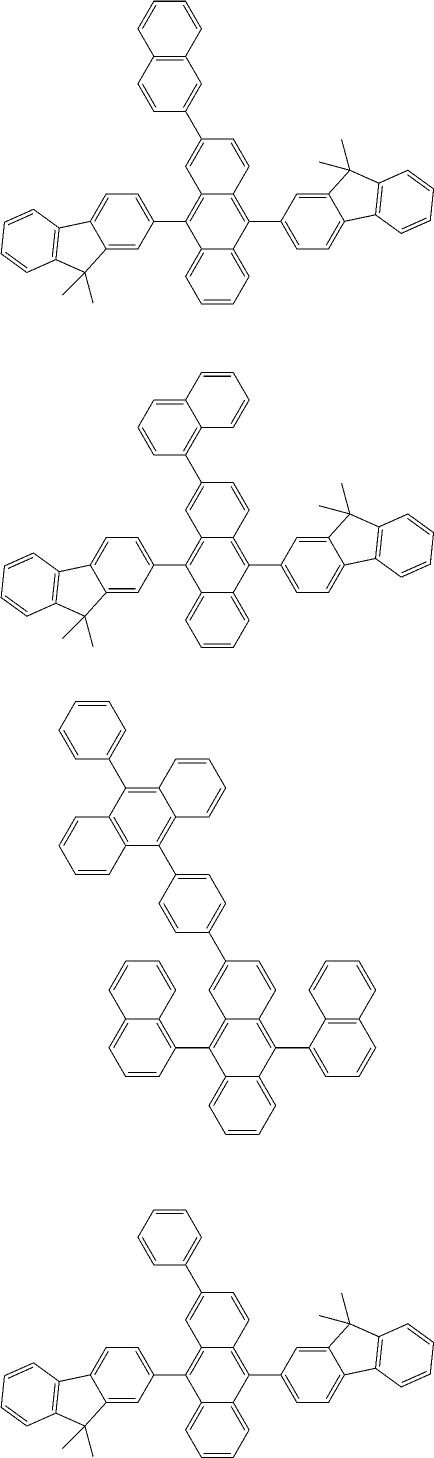

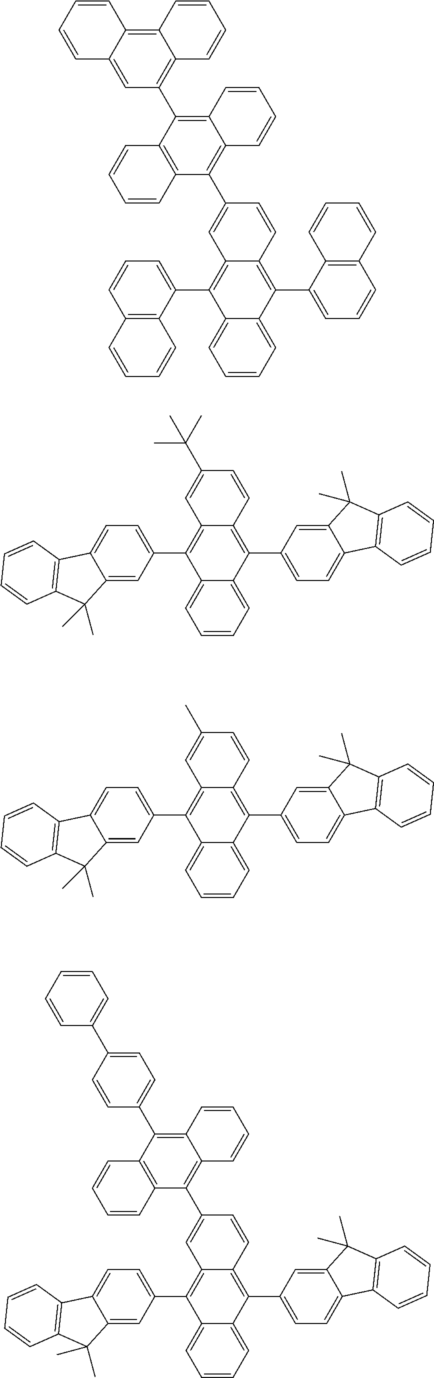

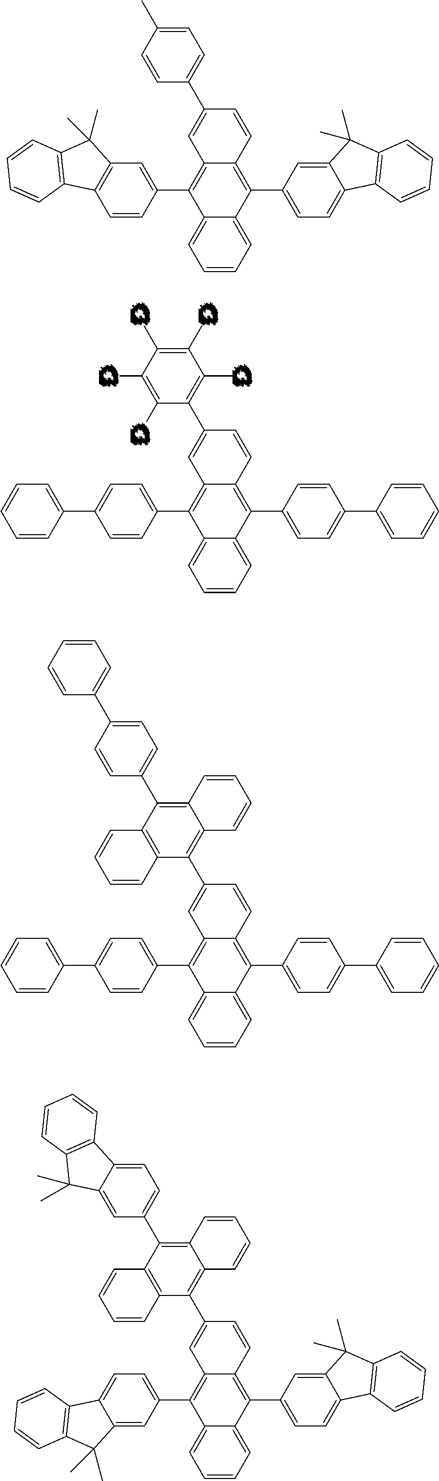

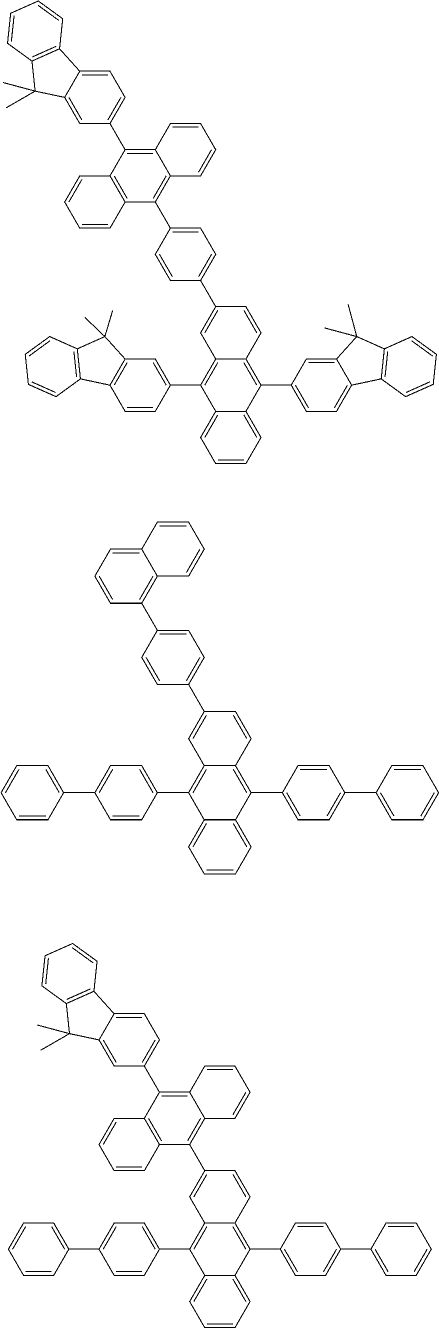

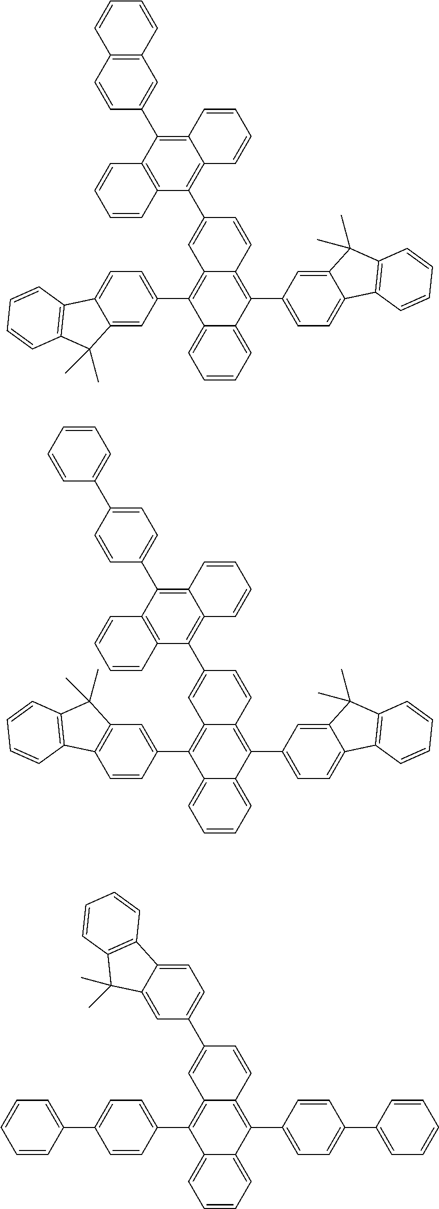

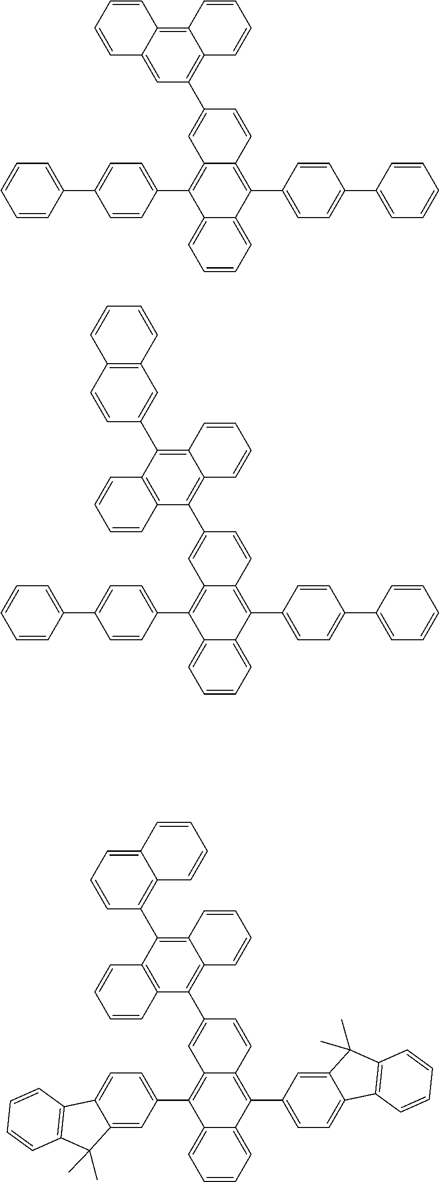

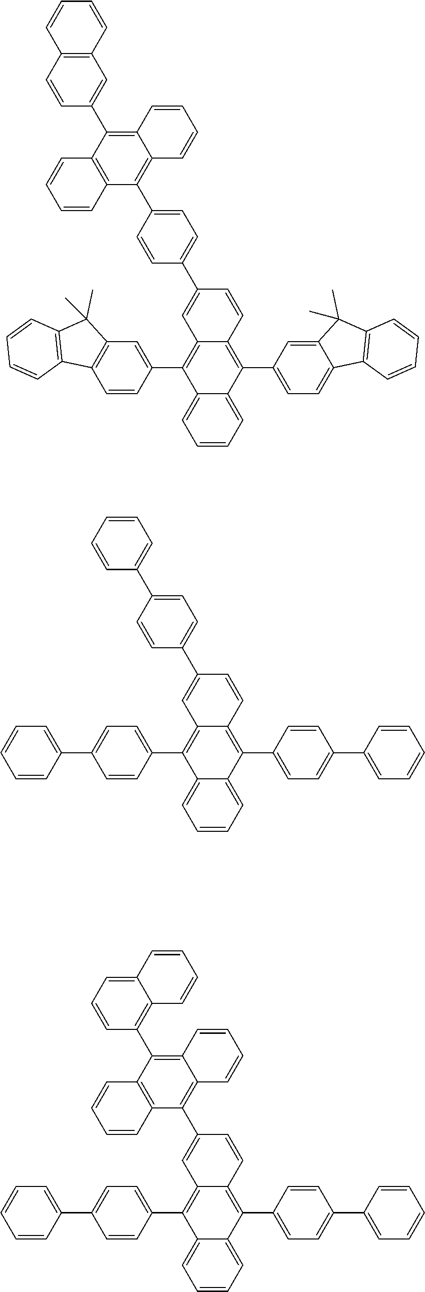

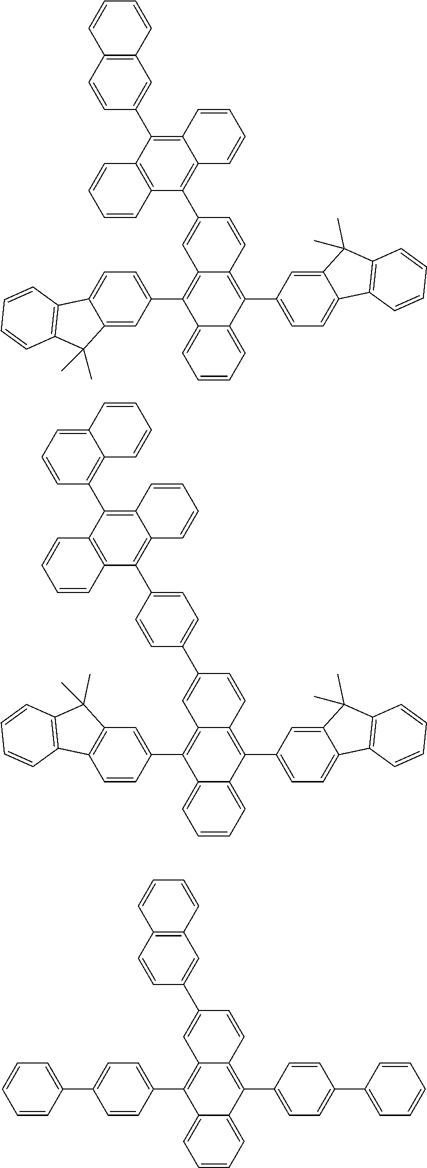

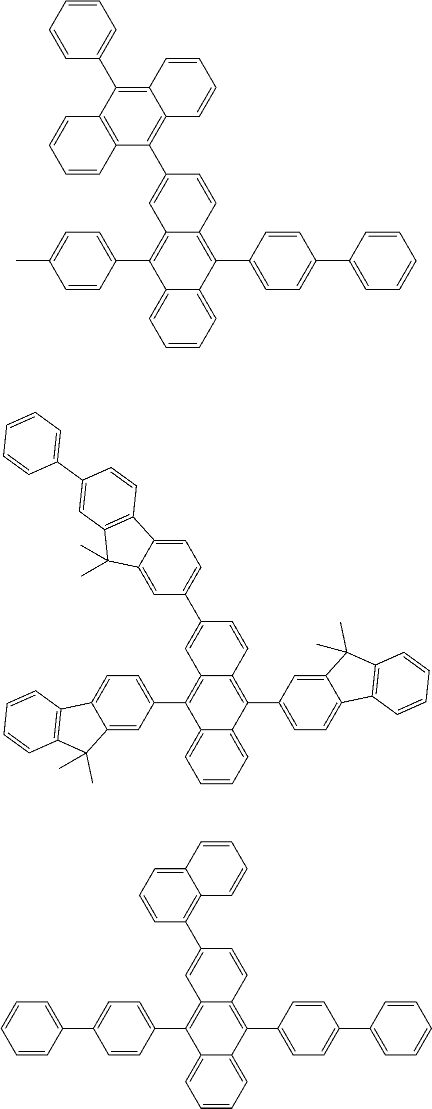

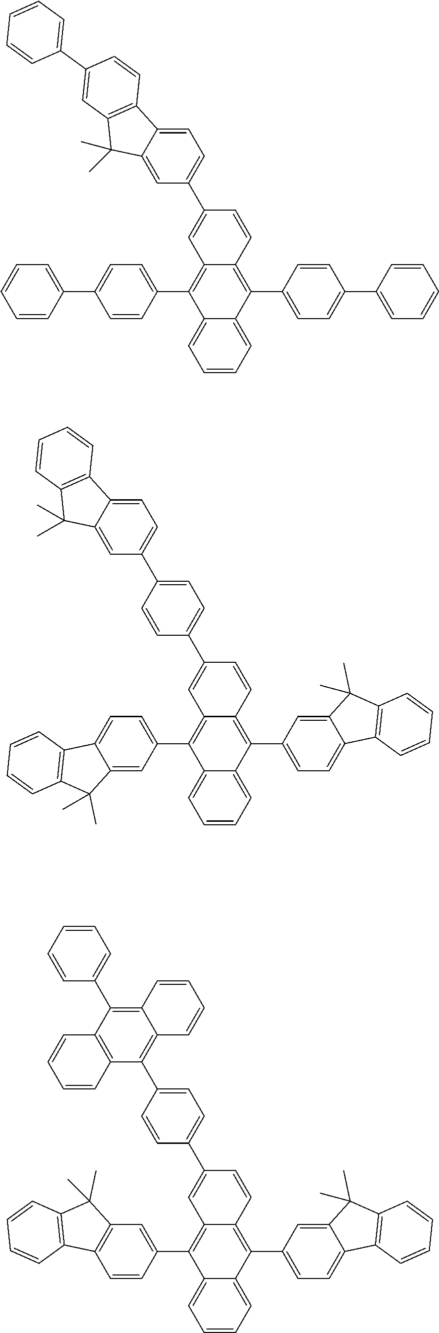

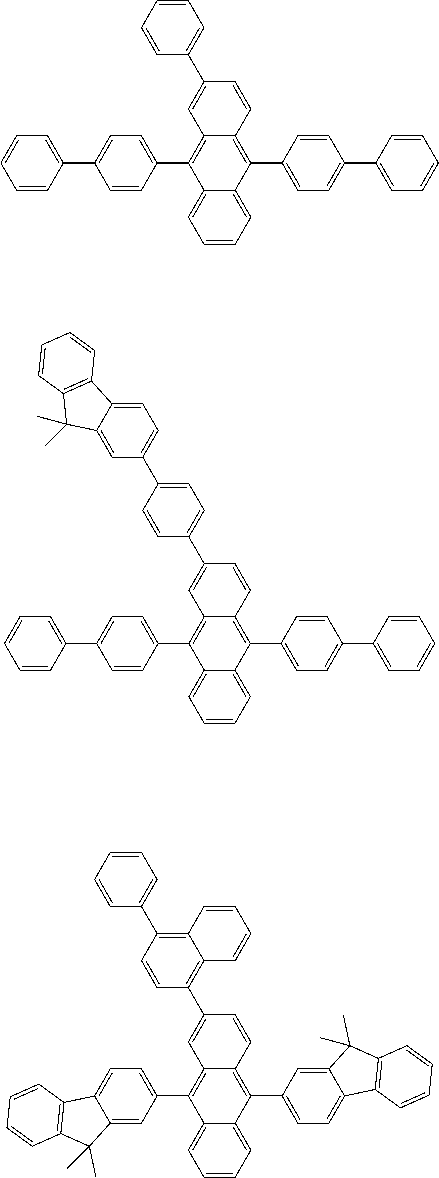

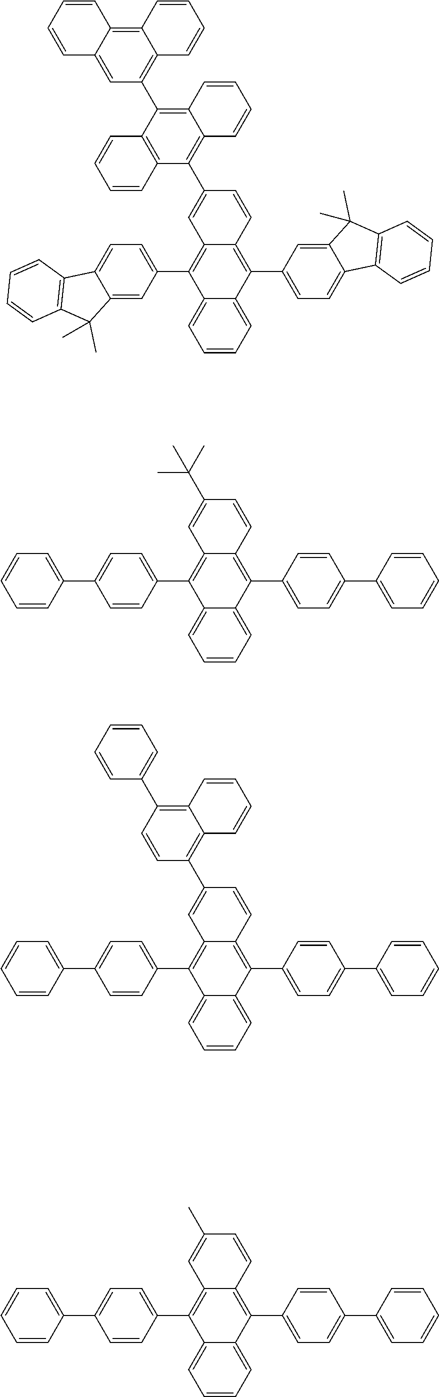

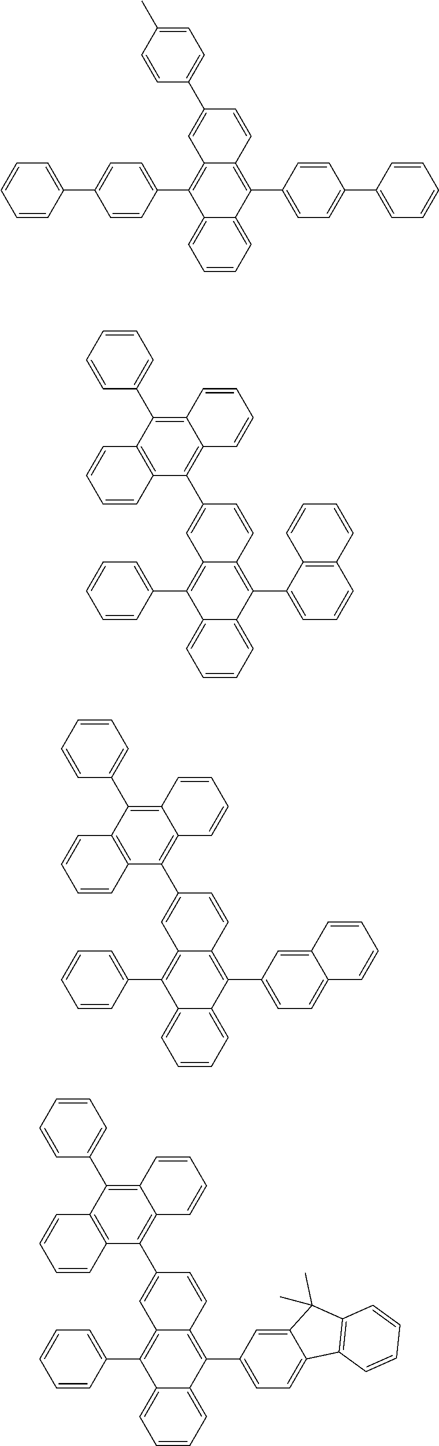

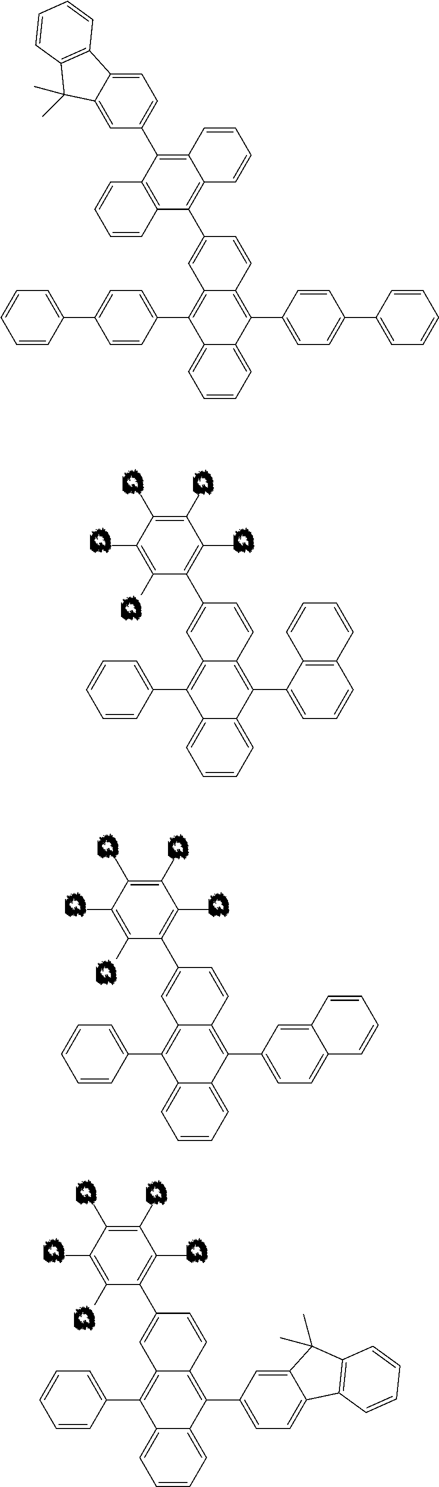

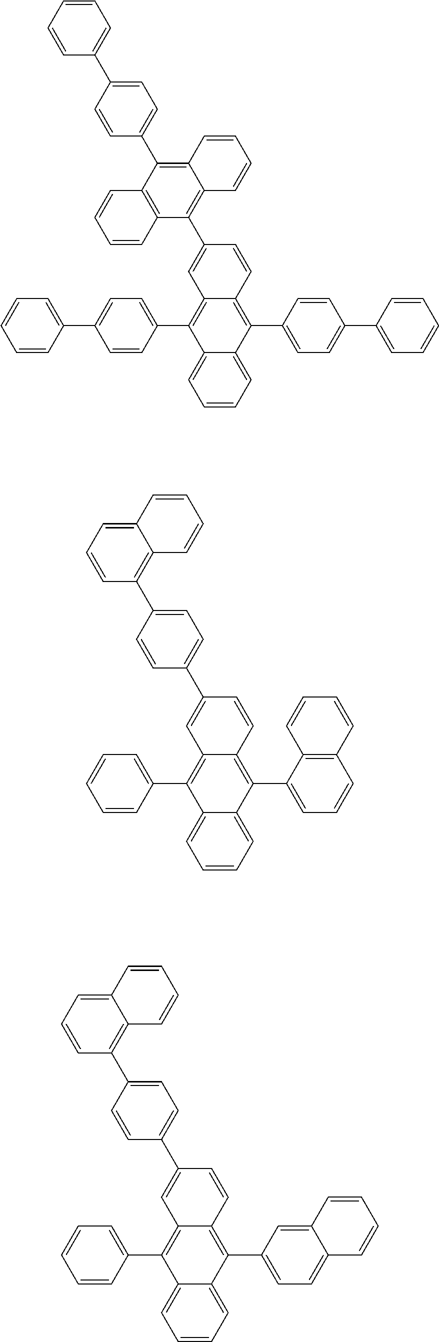

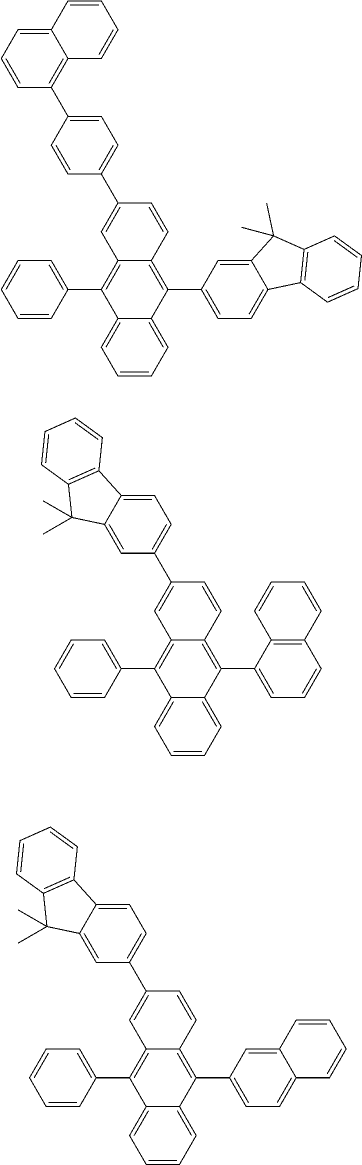

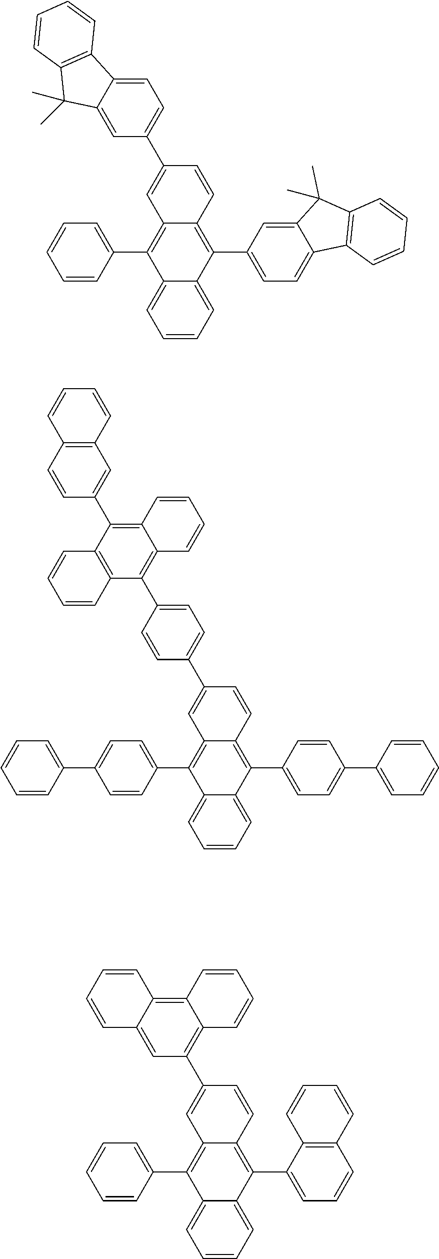

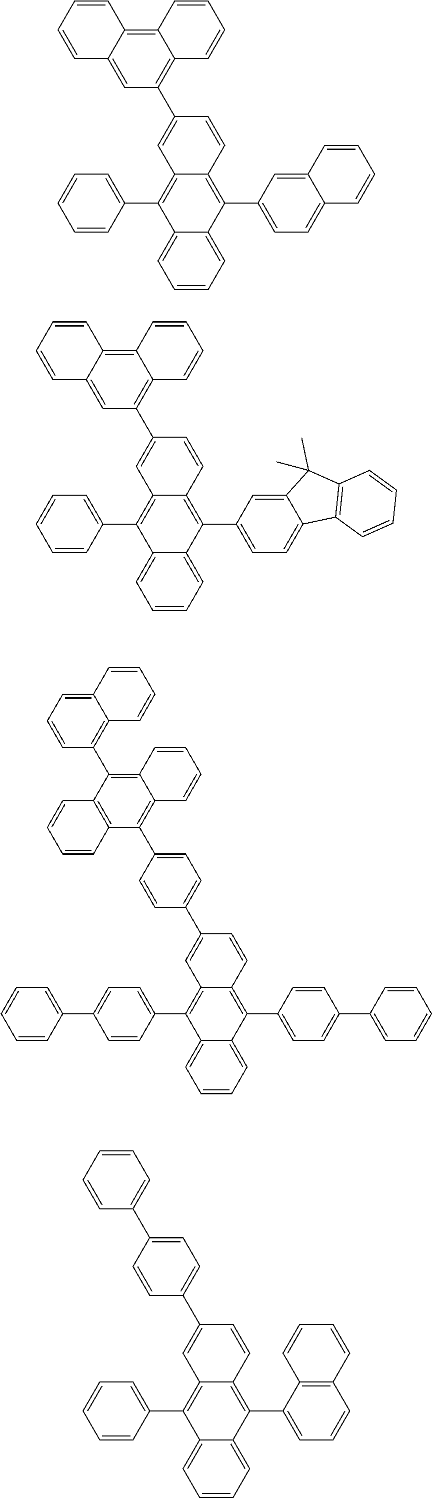

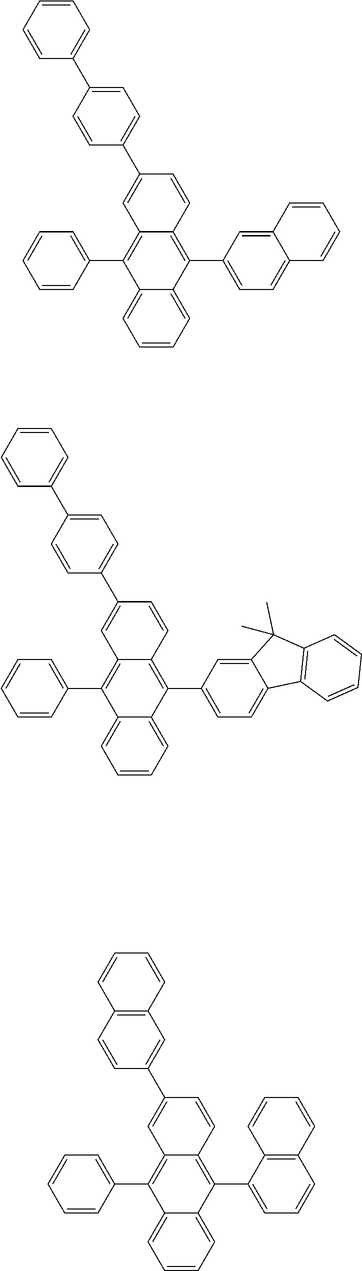

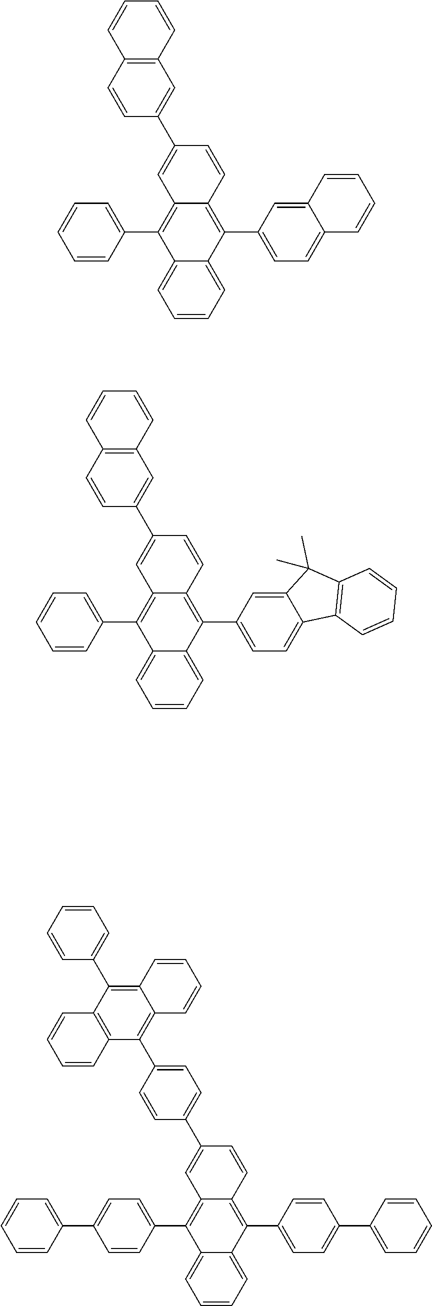

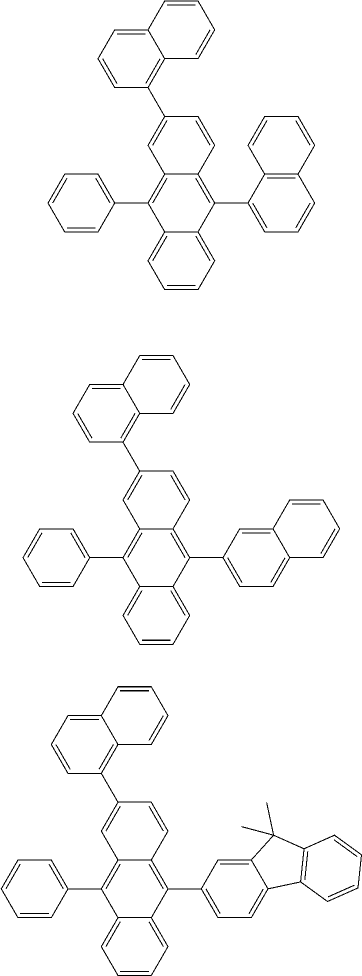

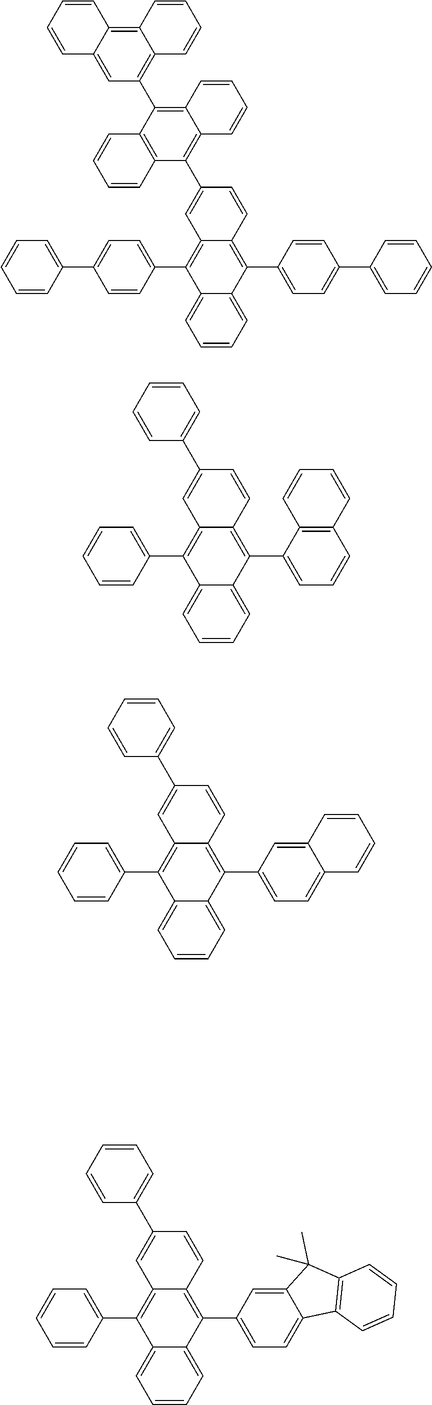

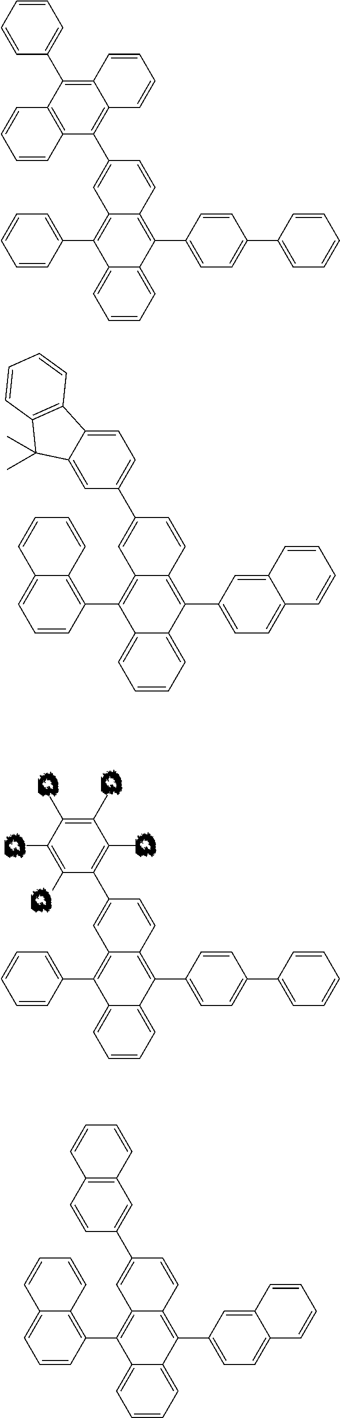

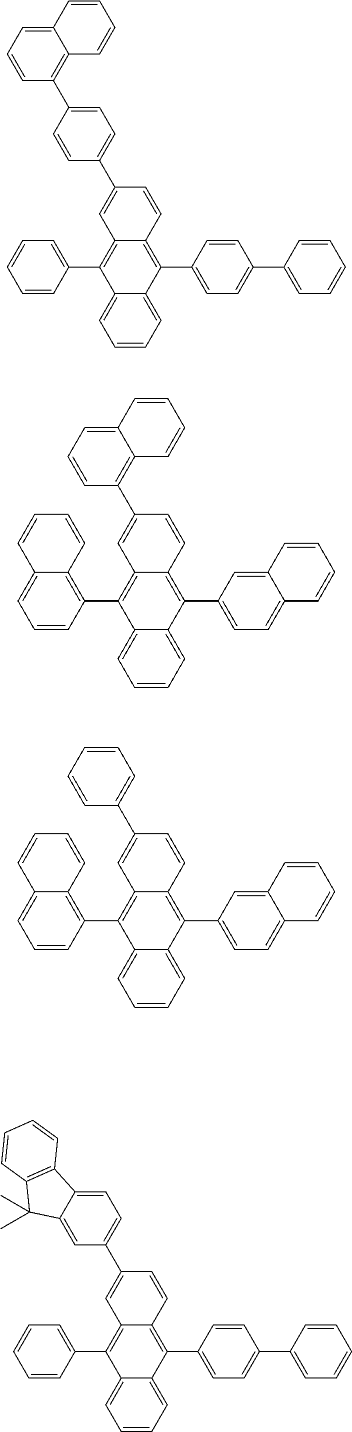

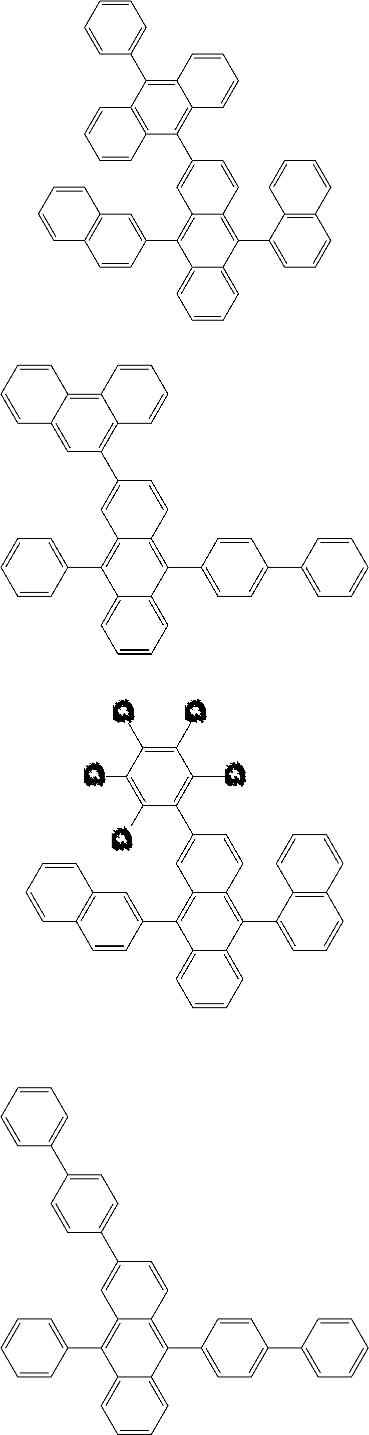

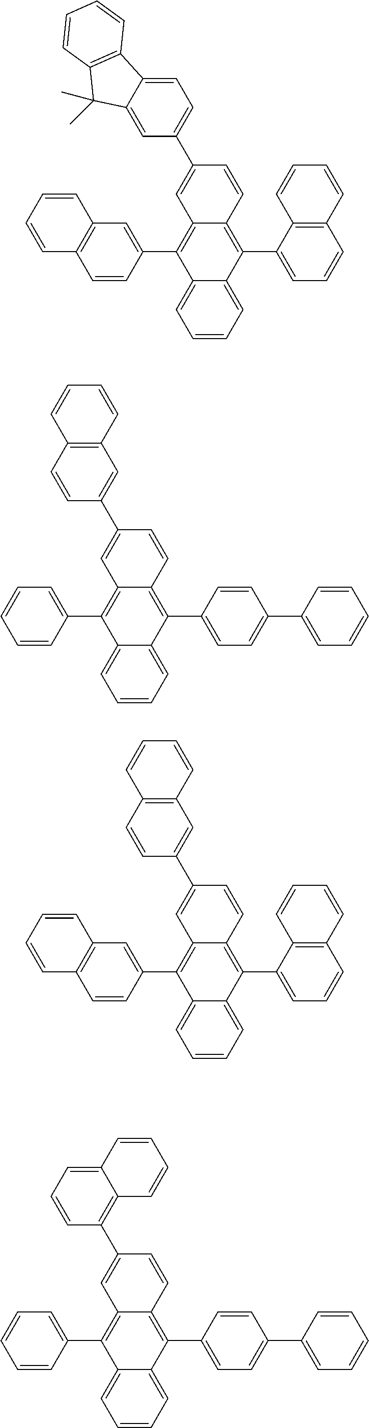

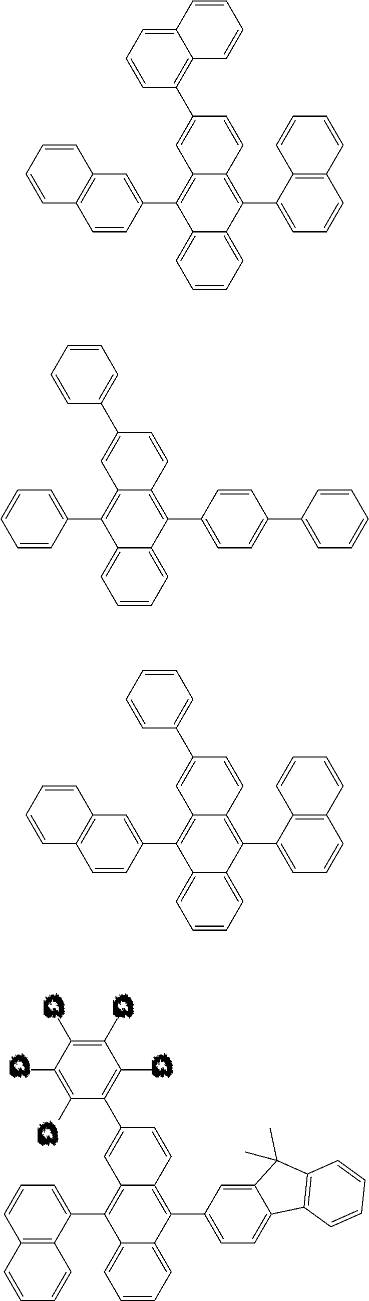

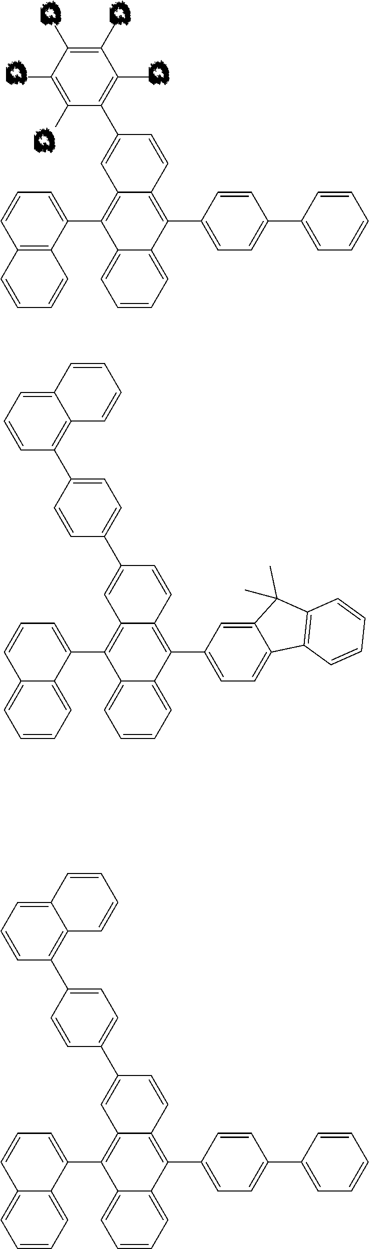

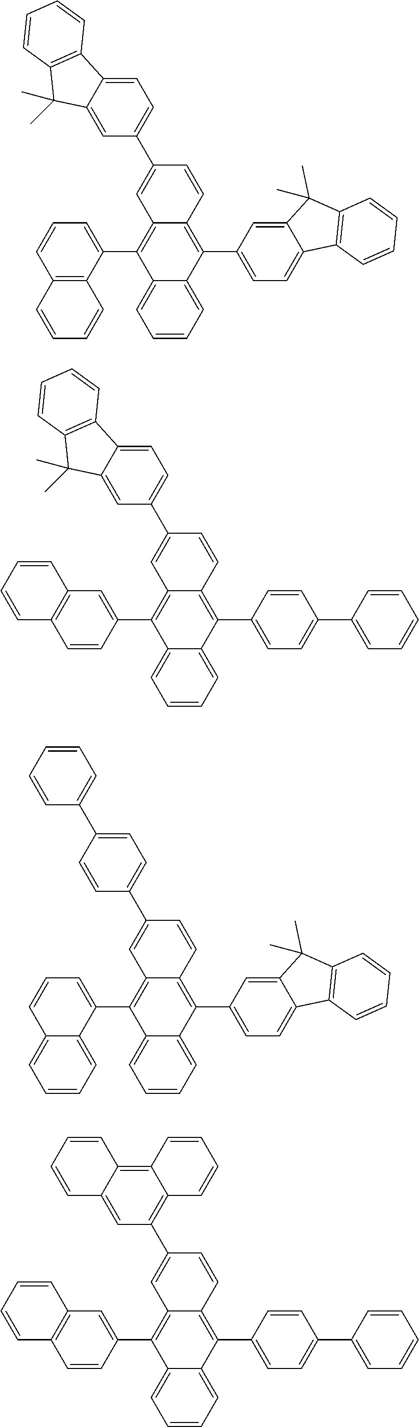

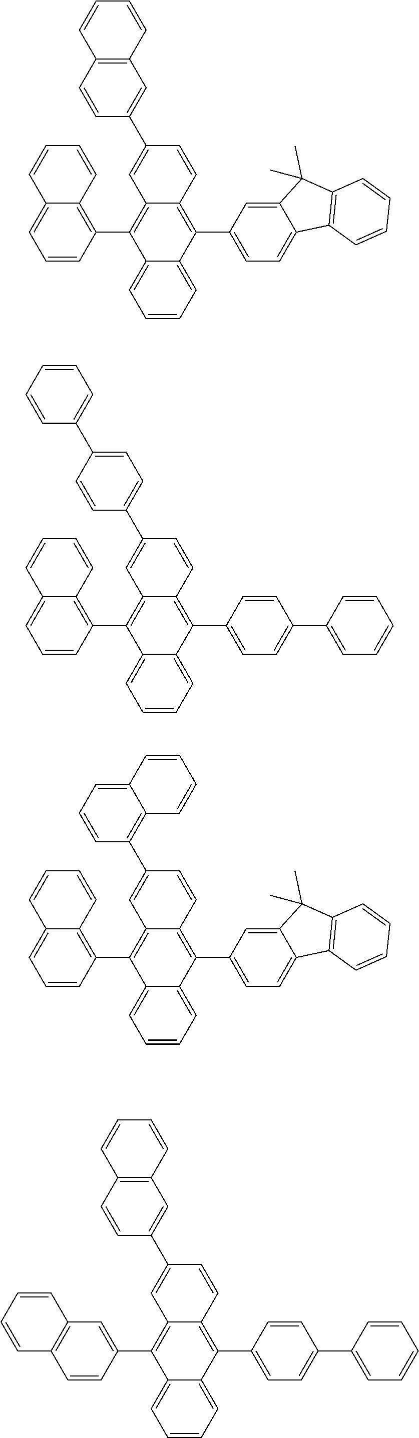

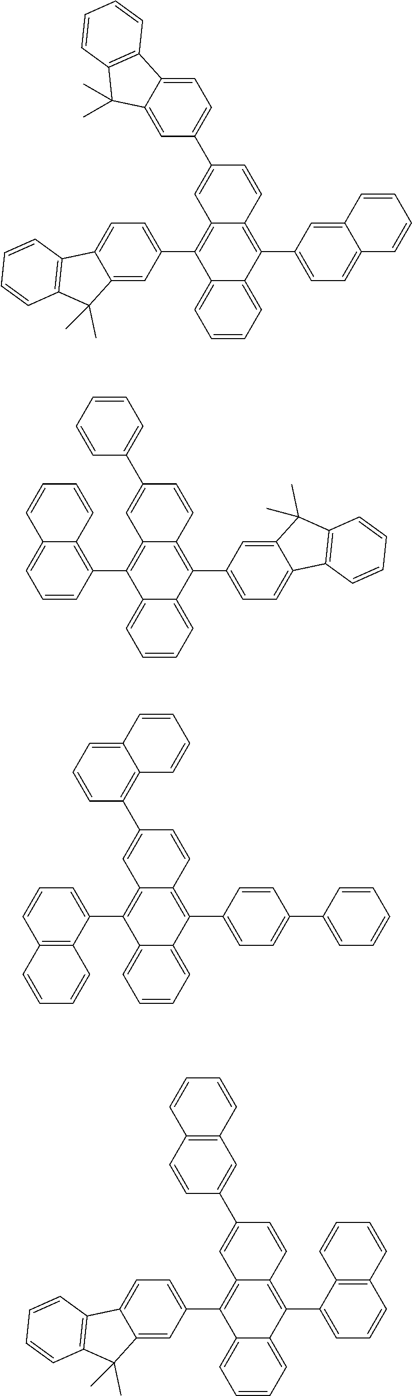

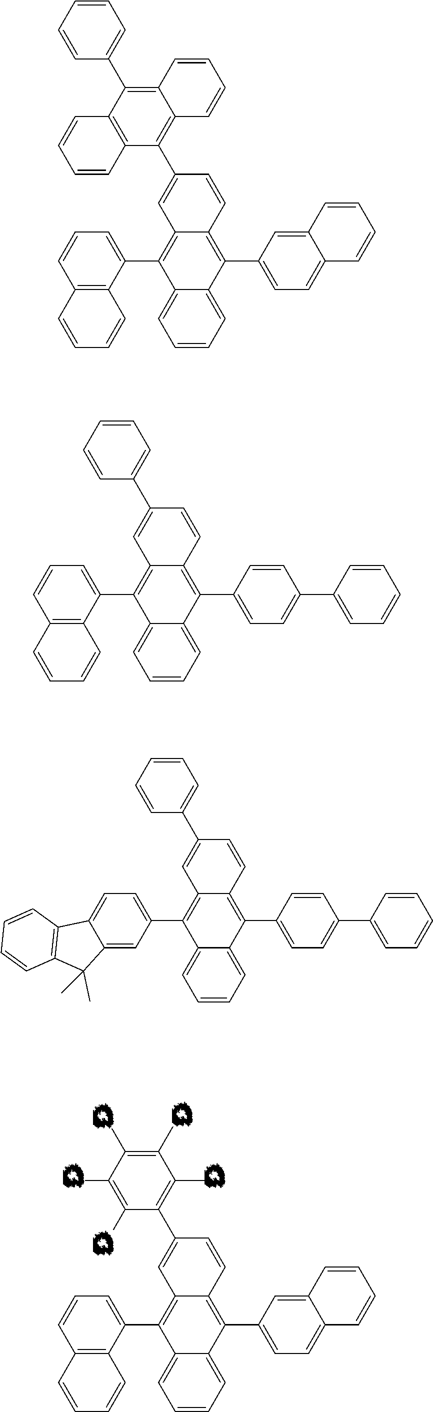

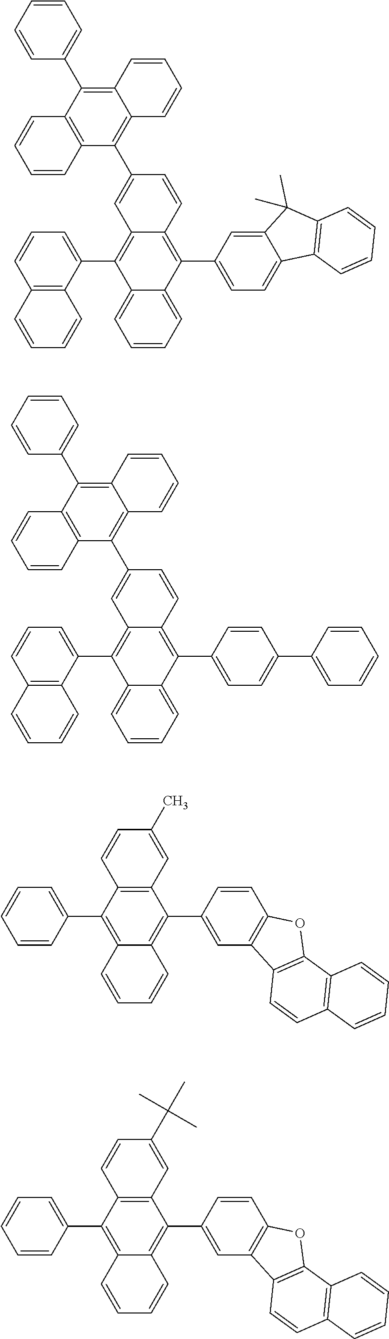

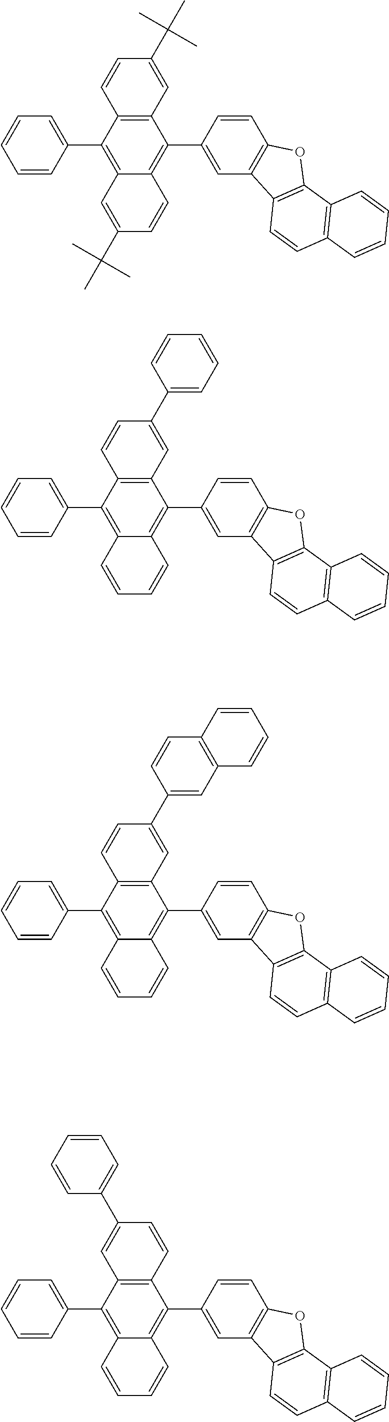 [SMILES: CC(C)(C)c1ccc2c(-c3ccc4oc5c6ccccc6ccc5c4c3)c3cc(C(C)(C)C)ccc3c(-c3ccccc3)c2c1 ; c1ccc(-c2ccc3c(-c4ccccc4)c4ccccc4c(-c4ccc5oc6c7ccccc7ccc6c5c4)c3c2)cc1 ; c1ccc(-c2c3ccccc3c(-c3ccc4oc5c6ccccc6ccc5c4c3)c3cc(-c4ccc5ccccc5c4)ccc23)cc1 ; c1ccc(-c2ccc3c(-c4ccc5oc6c7ccccc7ccc6c5c4)c4ccccc4c(-c4ccccc4)c3c2)cc1]

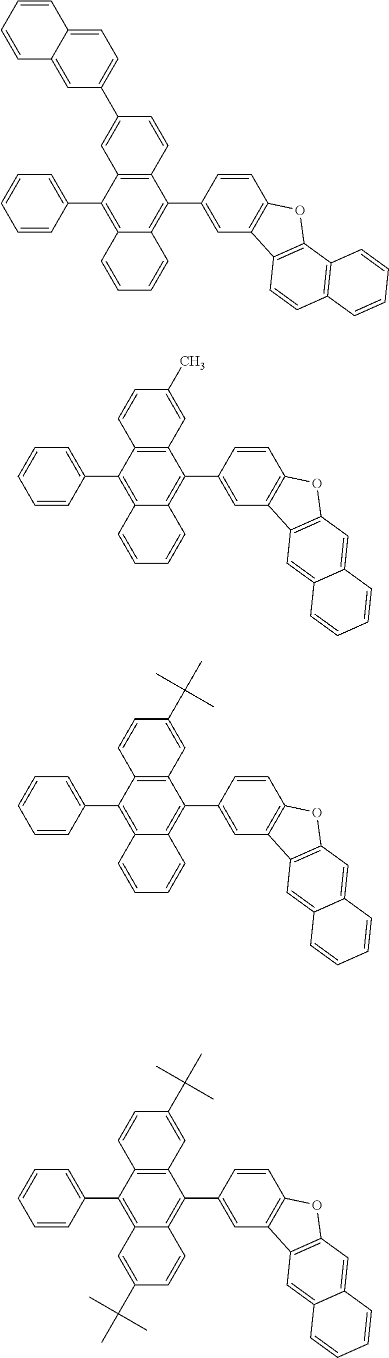 [SMILES: c1ccc(-c2c3ccccc3c(-c3ccc4oc5c6ccccc6ccc5c4c3)c3ccc(-c4ccc5ccccc5c4)cc23)cc1 ; Cc1ccc2c(-c3ccccc3)c3ccccc3c(-c3ccc4oc5cc6ccccc6cc5c4c3)c2c1 ; CC(C)(C)c1ccc2c(-c3ccccc3)c3ccccc3c(-c3ccc4oc5cc6ccccc6cc5c4c3)c2c1 ; CC(C)(C)c1ccc2c(-c3ccc4oc5cc6ccccc6cc5c4c3)c3cc(C(C)(C)C)ccc3c(-c3ccccc3)c2c1]

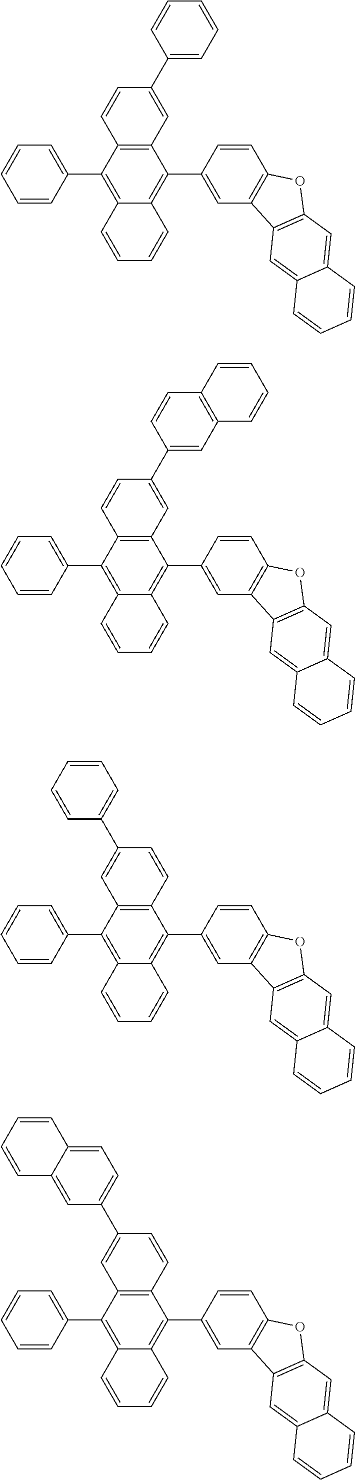 [SMILES: c1ccc(-c2ccc3c(-c4ccccc4)c4ccccc4c(-c4ccc5oc6cc7ccccc7cc6c5c4)c3c2)cc1 ; c1ccc(-c2c3ccccc3c(-c3ccc4oc5cc6ccccc6cc5c4c3)c3cc(-c4ccc5ccccc5c4)ccc23)cc1 ; c1ccc(-c2ccc3c(-c4ccc5oc6cc7ccccc7cc6c5c4)c4ccccc4c(-c4ccccc4)c3c2)cc1 ; c1ccc(-c2c3ccccc3c(-c3ccc4oc5cc6ccccc6cc5c4c3)c3ccc(-c4ccc5ccccc5c4)cc23)cc1]

In one embodiment, when the emitting layer contains the compound represented by the formula (1) and the compound represented by the formula (2), a content of the compound represented by the formula (1) is preferably 1% by mass or more and 20% by mass or less based on the total mass of the emitting layer.

In one embodiment, when the emitting layer contains the compound represented by the formula (1) and the compound represented by the formula (2), a content of the compound represented by the formula (2) is preferably 80% by mass or more and 99% by mass or less based on the total mass of the emitting layer.

### [Organic EL Device]

The organic EL device according to an aspect of the invention includes a cathode; an anode; and an organic layer provided therebetween; wherein the organic layer contains an emitting layer; and at least one layer of the organic layer contains a compound (1).

Hereinafter, a layer configuration of the organic EL device according to an aspect of the invention will be described.

The organic EL device according to an aspect of the invention has an organic layer between a pair of electrodes formed of the cathode and the anode. The organic layer contains at least one layer composed of an organic compound. AItematively, the organic layer is formed by stacking a plurality of layers composed of an organic compound. The organic layer may further contain an inorganic compound in addition to the organic compound.

In one embodiment, at least one layer of the organic layers is the emitting layer. The organic layer may be formed, for example, as one layer of the emitting layer, or may contain other layers which can be adopted in the layer configuration of the organic EL device. Examples of the layers that can be adopted in the layer configuration of the organic EL device are not particularty limited, and include, for example, a hole transport zone (a hole-transporting layer, a hole-injecting layer, an electron-blocking layer, an exciton-blocking layer, etc.) provided between the anode and the emitting layer, an emitting layer, a spacing layer, and an electron transport zone (an electron-transporting layer, an electron-injecting layer, a hole-blocking layer, etc.) provided between the cathode and the emitting layer.

The organic EL element according to an aspect of the invention may be, for example, a monochromatic light emitting element of a fluorescent or phosphorescent type, or a white light emitting element of a fluorescent/phosphorescent hybrid type. In addition, it may be a simple type containing a single light emitting unit or a tandem type containing a plurality of light emitting units.

The “light-emitting unit” described in the present specification refers to the smallest unit that contains an organic layer, at least one of the organic layers is an emitting layer, and emits light by recombination of injected hole and electron.

The “emitting layer” described in the present specification is an organic layer having a light emitting function. The emitting layer is, for example, a phosphorescent emitting layer, a fluorescent emitting layer, or the like, and may be a single layer or a plurality of layers.

The light-emitting unit may be of a laminated type containing a plurality of phosphorescent emitting layer and fluorescent emitting layer, and in this case, for example, may contain a spacing layer between each emitting layer for preventing excitons generated by the phosphorescent emitting layer from diffusing into the fluorescent emitting layer.

The simple type organic EL element includes, for example, a device configuration such as an anode, an emitting unit, or a cathode.

A typical layer configuration of the emitting unit is shown below. The layers in parentheses are optional.

(a) (hole-injecting layer/hole-transporting layer/fluorescent emitting layer (/electron-transporting layer/electron-injecting layer)
(b) (hole-injecting layer/hole-transporting layer/phosphorescent emitting layer (/electron-transporting layer/electron-injecting layer)
(c) (hole-injecting layer/hole-transporting layer/first fluorescent emitting layer/second fluorescent emitting layer (/electron-transporting layer/electron-injecting layer)
(d) (hole-injecting layer/hole-transporting layer/first phosphorescent layer/second phosphorescent layer (/electron-transporting layer/electron-injecting layer)
(e) (hole-injecting layer/hole-transporting layer/phosphorescent emitting layer/spacing layer/fluorescent emitting layer (/electron-transporting layer/electron-injecting layer)
(f) (hole-injecting layer/hole-transporting layer/first phosphorescent emitting layer/second phosphorescent emitting layer/spacing layer/fluorescent emitting layer (/electron-transporting layer/electron-injecting layer)
(g) (hole-injecting layer/hole-transporting layer/first phosphorescent layer/spacing layer/second phosphorescent emitting layer/spacing layer/fluorescent emitting layer (/electron-transporting layer/electron-injecting layer)
(h) (hole-injecting layer/hole-transporting layer/phosphorescent emitting layer/spacing layer/first fluorescent emitting layer/second fluorescent emitting layer (/electron-transporting layer/electron-injecting layer)
(i) (hole-injecting layer/hole-transporting layer/electron-blocking layer/fluorescent emitting layer (/electron-transporting layer/electron-injecting layer)
(j) (hole-injecting layer/hole-transporting layer/electron-blocking layer/phosphorescent emitting layer (/electron-transporting layer/electron-injecting layer)
(k) (hole-injecting layer/hole-transporting layer/exciton-blocking layer/fluorescent emitting layer (/electron-transporting layer/electron-injecting layer)
(l) (hole-injecting layer/hole-transporting layer/exciton-blocking layer/phosphorescent emitting layer (/electron-transporting layer/electron-injecting layer)
(m) (hole-injecting layer/first hole-transporting layer/second hole-transporting layer/fluorescent emitting layer (/electron-transporting layer/electron-injecting layer)
(n) (hole-injecting layer/first hole-transporting layer/second hole-transporting layer/fluorescent emitting layer (/first electron-transporting layer/second electron-transporting layer/electron-injection layer)
(o) (hole-injecting layer/first hole-transporting layer/second hole-transporting layer/phosphorescent emitting layer (/electron-transporting layer/electron-injecting layer)
(p) (hole-injecting layer/first hole-transporting layer/second hole-transporting layer/phosphorescent emitting layer (/first electron-transporting layer/second electron-transporting layer/electron-injecting layer)
(q) (hole-injecting layer/hole-transporting layer/fluorescent emitting layer/hole-blocking layer (/electron-transporting layer/electron-injecting layer)
(r) (hole-injecting layer/hole-transporting layer/phosphorescent emitting layer/hole-blocking layer(/electron-transport layer/electron-injecting layer)
(s) (hole-injecting layer/hole-transporting layer/fluorescent emitting layer/exciton-blocking layer (/electron-transporting layer/electron-injecting layer)
(t) (hole-injecting layer/hole-transporting layer/phosphorescent emitting layer/exciton-blocking layer (/electron-transporting layer/electron-injecting layer)

The layer structure of the organic EL device according to an aspect of the invention is not limited thereto. For example, when the organic EL device has a hole-injecting layer and a hole-transporting layer, it is preferred that a hole-injecting layer be provided between the hole-transporting layer and the anode. Further, when the organic EL device has an electron-injecting layer and an electron-transporting layer, it is preferred that an electron-injecting layer be provided between the electron-transporting layer and the cathode. Further, each of the hole-injecting layer, the hole-transporting layer, the electron-transporting layer and the electron-injecting layer may be formed of a single layer or be formed of a plurality of layers.

The plurality of phosphorescent emitting layers, and the plurality of the phosphorescent emitting layer and the fluorescent emitting layer may be emitting layers that emit mutually different colors. For example, the emitting unit (f) may contain a hole-transporting layer/first phosphorescent layer (red light emission)/second phosphorescent emitting layer (green light emission)/spacing layer/fluorescent emitting layer (blue light emission)/electron-transporting layer.

An electron-blocking layer may be provided between each light emitting layer and the hole-transporting layer or the spacing layer. Further, a hole-blocking layer may be provided between each emitting layer and the electron-transporting layer. By providing the electron-blocking layer or the hole-blocking layer, it is possible to confine electrons or holes in the emitting layer, thereby to improve the recombination probability of carriers in the emitting layer, and to improve light emitting efficiency.

As a representative device configuration of a tandem type organic EL device, for example, a device configuration such as anode/first emitting unit/intermediate layer/second emitting unit/cathode can be given.

The first emitting unit and the second emitting unit are independently selected from the above-mentioned emitting units, for example.

The intermediate layer is also generally referred to as an intermediate electrode, an intermediate conductive layer, a charge generating layer, an electron withdrawing layer, a connecting layer, a connector layer, or an intermediate insulating layer. The intermediate layer is a layer that supplies electrons to the first emitting unit and holes to the second emitting unit, and can be formed from known materials.

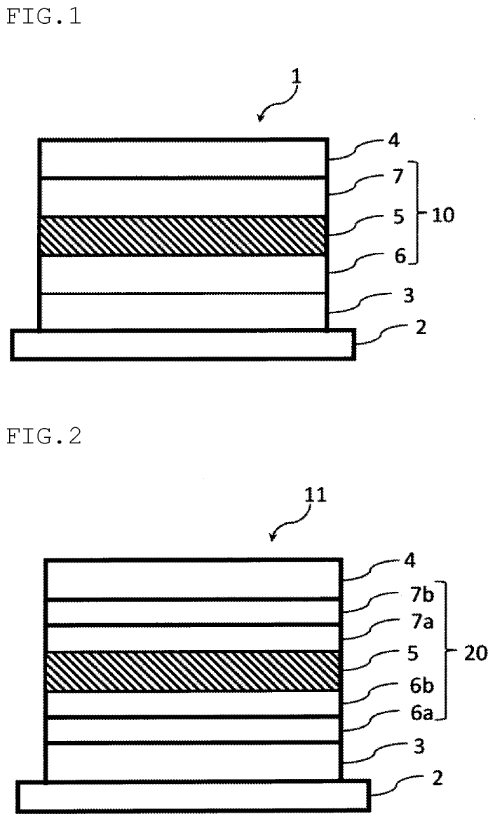

FIG. 1 shows a schematic view of one example of the layer structure of the organic EL device. An organic EL device 1 has a substrate 2, an anode 3, a cathode 4, and an emitting unit (organic layer) 10 arranged between the anode 3 and the cathode 4. The emitting unit 10 has at least one emitting layer 5.

A hole-transporting zone (hole-injecting layer, hole-transporting layer, etc.) 6 may be formed between the emitting layer 5 and the anode 3, and an electron-transporting zone (electron-injecting layer, electron-transporting layer, etc.) 7 may be formed between the emitting layer 5 and the cathode 4. An electron-blocking layer (not shown) may be provided on the anode 3 side of the emitting layer 5, and a hole-blocking layer (not shown) may be provided on the cathode 4 side of the emitting layer 5. Due to such a configuration, electrons or holes are confined in the emitting layer 5, whereby efficiency of formation of excitons in the emitting layer 5 can be further enhanced.

FIG. 2 schematically shows another example of the layer structure of the organic EL element. FIG. 2 shows a schematic view of another example of the layer configuration of the organic EL device. In an organic EL device 11 shown in FIG. 2, in an emitting unit 20, the hole-transporting layer in the hole-transporting zone 6 and the electron-transporting layer in the electron-transporting zone 7 of the emitting unit 10 of the organic EL device 1 in FIG. 1 are respectively composed of two layers. The hole-transporting zone 6 has a first hole-transporting layer 6a on the anode side and a second hole-transporting layer 6b on the cathode side. The electron-transporting zone 7 has a first electron-transporting layer 7a on the anode side and a second hole-transporting layer 7b on the cathode side. As forthe other numerical references, since they are the same as those in FIG. 1, an explanation is omitted.

Hereinbelow, an explanation will be made on function, materials, etc. of each layer constituting the organic EL device described in the present specification.

### (Substrate)

The substrate is used as a support of the organic EL device. The substrate preferably has alight transmittance of 50% or more in the visible light region with a wavelength of 400 to 700 nm, and a smooth substrate is preferable. Examples of the material of the substrate include soda-lime glass, aluminosilicate glass, quartz glass, plastic and the like. Asa substrate, a flexible substrate can be used. The flexible substrate refers to a flexible substrate, for example, a plastic substrate or the like. The flexible substrate means a substrate that can be bent (flexible), and examples thereof include a plastic substrate and the like. Specific examples of the material for forming the plastic substrate include polycarbonate, polyallylate, polyethersulfone, polypropylene, polyester, polyvinyl fluoride, polyvinyl chloride, polyimide, polyethylene naphthalate and the like. Also, an inorganic vapor deposited film can be used.

### (Anode)

As the anode, for example, it is preferable to use a metal, an alloy, a conductive compound, a mixture thereof or the like and having a high work function (specifically, 4.0 eV or more). Specific examples of the material of the anode include indium oxide-tin oxide (ITO: Indium Tin Oxide), indium oxide-tin oxide containing silicon or silicon oxide, indium oxide-zinc oxide, indium oxide containing tungsten oxide or zinc oxide, graphene and the like. In addition, it is also possible to use gold, silver, platinum, nickel, tungsten, chromium, molybdenum, iron, cobalt, copper, palladium, titanium, and nitrides of these metals (e.g. titanium oxide).

The anode is normally formed by depositing these materials on the substrate by a sputtering method. For example, indium oxide-zinc oxide can be formed by a sputtering method by using a target in which 1 to 10 mass % zinc oxide is added relative to indium oxide. Further, indium oxide containing tungsten oxide or zinc oxide can be formed by a sputtering method by using a target in which 0.5 to 5 mass % of tungsten oxide or 0.1 to 1 mass % of zinc oxide is added relative to indium oxide.

As the other methods forforming the anode, a vacuum deposition method, a coating method, an inkjet method, a spin coating method or the like can be given. When silver paste or the like is used, it is possible to use a coating method, an inkjet method or the like.

The hole-injecting layer formed in contact with the anode is formed by using a material that allows easy hole injection regardless of the work function of the anode. For this reason, in the anode, it is possible to use a common electrode material, e.g. a metal, an alloy, a conductive compound and a mixture thereof. Specifically, a material having a small work function such as alkaline metals such as lithium and cesium; alkaline earth metals such as calcium and strontium; alloys containing these metals (for example, magnesium-silver and aluminum-lithium); rare earth metals such as europium and ytterbium; and an alloy containing rare earth metals.

### (Hole-Injecting Layer)

A hole-injecting layer is a layerthat contains a substance having high hole-injection property and has a function of injecting holes from the anode to the organic layer. As the substance having high hole-injection property, molybdenum oxide, titanium oxide, vanadium oxide, rhenium oxide, ruthenium oxide, chromium oxide, zirconium oxide, hafnium oxide, tantalum oxide, silver oxide, tungsten oxide, manganese oxide, an aromatic amine compound, an electron-attracting (acceptor) compound or a polymeric compound (oligomer, dendrimer, polymer, etc.) orthe like can be used. Among these, a compound such as an aromatic amine compound and an acceptor compound are preferable, with an acceptor compound being more preferable.

As specific examples of an aromatic amine compound, 4,4′, 4″-tris(N,N-diphenylamino)triphenylamine (abbreviation: TDATA), 4,4′, 4″-tris[N-(3-methylphenyl-N-phenylamino] triphenylamine (abbreviation: MTDATA), 4,4′-bis[N-(4-diphenylaminophenyl-N-phenylamino] biphenyl (abbreviation: DPAB), 4,4′-bis(N-{4-[N′-(3-methylphenyl-N′-phenylamino]phenyl}-N-phenylamino)biphenyl (abbreviation: DNTPD),1,3,5-tris[N-(4-diphenylaminophenyl-N-phenylamino]benzene (abbreviation: DPA3B), 3-[N-(9-phenylcarbazol-3-yl-N-phenylamino]-9-phenylcarbazole (abbreviation: PCzPCA1), 3,6-bis[N-(9-phenylcarbazol-3-yl-N-phenylamino]-9-phenylcarbazole (abbreviation: PCzPCA2), 3-[N-(1-naphthyl-N-(9-phenylcarbazol-3-ylamino]-9-phenylcarbazole (abbreviation: PCzPCN1) orthe like can be given.

As the acceptor compound, for example, a heterocyclic derivative having an electron attracting group, a quinone derivative having an electron attracting group, an aryl borane derivative, a heteroaryl borane derivative and the like are preferable. As specific examples, hexacyanohexaazatriphenylene, 2,3,5,6-tetrafluoro-7,7,8,8-tetracyanoquinodimethane (abbreviation: F4TCNQ),1,2,3-tris[(cyano)(4-cyano-2,3,5,6-tetrafluorophenylmethylene]cydopropane orthe like can be given. When the acceptor compound is used, it is preferred that the hole-injecting layer further comprise a matrix material. As the matrix material, a material known as the material for an organic EL device can be used. For example, an electron-donating (donor) compound can be used. More preferably, the above-mentioned aromatic amine compound can be used.

### (Hole-Transporting Layer)

The hole-transporting layer is a layerthat comprises a high hole-transporting property, and has a function of transporting holes from the anode to the organic layer.

As the substance having a high hole-transporting property, a material having a hole mobility of 10⁻⁶ cm²/(Vs) or more is preferable. For example, aromatic amine compounds, carbazole derivatives, anthracene derivatives, polymeric compounds, and the like can be given, for example.

Specific examples of the aromatic amine compound include 4,4′-bis[N-(1-naphthyl-N-phenylamino]biphenyl (abbreviation: NPB), N,N′-bis(3-methylphenyl)-N,N′-diphenyl-[1,1′-biphenyl]-4,4′-diamine (abbreviation: TPD), 4-phenyl-4′-(9-phenylfluorene-9-yl)triphenylamine (abbreviation: BAFLP), 4,4′-bis[N-(9,9-dimethylfluorene-2-yl)-N-phenylamino]biphenyl (abbreviation: DFLDPBi), 4,4′,4″-tris(N,N-diphenylamino)triphenylamine (abbreviation: TDATA), 4,4′,4″-tris[N-(3-methylpheny)-N-phenylamino]triphenylamine (abbreviation: MTDATA), 4,4′-bis[N-(spiro-9,9′-bifluorene-2-yl)-N-phenylamino]biphenyl (abbreviation: BSPB) orthe like.

Specific examples of the carbazole derivatives include 4,4′-di(9-carbazoly)biphenyl (abbreviation: CBP), 9-[4-(9-carbazoly)phenyl]-10-phenylanthracene (abbreviation: CzPA), 9-phenyl-3-[4-(10-phenyl-9-anthry)phenyl]-9H-carbazole (abbreviation: PCzPA) or the like.

Specific examples of anthracene derivatives include 2-t-butyl-9,10-di(2-naphthy)anthracene (t-BuDNA), 9,10-di(2-naphthy)anthracene (DNAs), 9,10-diphenylanthracene (DPAnth), and the like.

Specific examples of the polymeric compound include poly(N-vinylcarbazole) (abbreviation: PVK) and poly(4-vinyltriphenylamine) (abbreviation: PVTPA).

As long as it is a compound having a higher hole-transporting property as compared with electron-transporting property, other substances than those mentioned above can be used.

The hole-transporting layer may be a single layer or may be a laminated layer of two or more layers. In this case, it is preferred that a layer that contains a substance having a larger energy gap among substances having higher hole-transporting property be arranged on a side nearer to the emitting layer.

### (Emitting Layer)

The emitting layer is a layer containing a substance having a high emitting property (dopant material). As the dopant material, various materials can be used. For example, a fluorescent emitting compound (fluorescent dopant), a phosphorescent emitting compound (phosphorescent dopant) or the like can be used. A fluorescent emitting compound is a compound capable of emitting light from the singlet excited state, and an emitting layer containing a fluorescent emitting compound is called a fluorescent emitting layer. Further, a phosphorescent emitting compound is a compound capable of emitting light from the triplet excited state, and an emitting layer containing a phosphorescent emitting compound is called a phosphorescent emitting layer.

The emitting layer normally contains a dopant material and a host material that allows it to emit light efficiently. In some literatures, a dopant material is called a guest material, an emitter or an emitting material. In some literatures, a host material is called a matrix material. Hosts material may also be referred to in some literature as matrices material.

A single emitting layer may comprise plural dopant materials and plural host materials. Further, plural emitting layers may be present.

In the present specification, a host material combined with the fluorescent dopant is referred to as a “fluorescent host” and a host material combined with the phosphorescent dopant is referred to as the “phosphorescent host.” Note that the fluorescent host and the phosphorescent host are not classified only by the molecular structure. The phosphorescent host is a material for forming a phosphorescent emitting layer containing a phosphorescent dopant, but does not mean that it cannot be used as a material for forming a fluorescent emitting layer. The same can be applied to the fluorescent host.

It is preferred that the emitting layer contain the compound represented by the formula (1). More preferably, it is contained as a dopant material. Further, it is preferred that the compound (1) be contained in the emitting layer as a fluorescent dopant.

The content of compound (1) in emitting layer as the dopant material is not particularly limited, but from the viewpoint of adequate luminescence and quenching of concentrations, it is preferable, for example, to be 0.1 to 70% by mass, more preferably 0.1 to 30 mass %, more preferably 1 to 30 mass %, still more preferably 1 to 20 mass %, and particularly preferably 1 to 10 mass %.

### <Fluorescent Dopant>

As the fluorescent dopant other than the compound (1), a fused polycyclic aromatic derivative, a styrylamine derivative, a fused ring amine derivative, a boron-containing compound, a pyrrole derivative, an indole derivative, a carbazole derivative can be given, for example. Among these, a fused ring amine derivative, a boron-containing compound, carbazole derivative is preferable.

As the fused ring amine derivative, a diaminopyrene derivative, a diaminochrysene derivative, a diaminoanthracene derivative, a diaminofluorene derivative, a diaminofluorene derivative with which one or more benzofuro skeletons are fused, or the like can be given.

As the boron-containing compound, a pyrromethene derivative, a triphenylborane derivative or the like can be given.

Examples of the blue fluorescent dopant include pyrene derivatives, styrylamine derivatives, chrysene derivatives, fluoranthene derivatives, fluorene derivatives, diamine derivatives, and triarylamine derivatives. As the blue fluorescent dopant, pyrene derivatives, styrylamine derivatives, chrysene derivatives, fluoranthene derivatives, fluorene derivatives, diamine derivatives, triarylamine derivatives and the like can be given, for example. Specifically, N,N′-bis[4-(9H-carbazol-9-ylphenyl]-N,N′-diphenylstilbene-4,4′-diamine (abbreviation: YGA2S), 4-(9H-carbazol-9-yl)-4′-(10-phenyl-9-anthryltriphenylamine (abbreviation: YGAPA), 4-(10-phenyl-9-anthryl-4′-(9-phenyl-9H-carbazole-3-yltriphenylamine (abbreviation: PCBAPA) orthe like can be given.

As the green fluorescent dopant, an aromatic amine derivative or the like can be given, for example. Specifically, N-(9,10-diphenyl-2-anthry)-N,9-diphenyl-9H-carbazole-3-amine (abbreviation: 2PCAPA), N-[9,10-bis(1,1′-biphenyl-2-yl-2-anthryl]-N,9-diphenyl-9H-carbazole-3-amine (abbreviation: 2PCABPhA), N-(9,10-diphenyl-2-anthryl-N,N′,N′-triphenyl-1,4-phenylenediamine (abbreviation:2DPAPA), N-[9,10-bis(1,1′-biphenyl-2-yl)-2-anthryl]-N,N′,N′-triphenyl-1,4-phenylenediamine (abbreviation:2DPABPhA), N-[9,10-bis(1,1′-biphenyl-2-yl]-N-[4-(9H-carbazole-9-ylphenyl]-N-phenylanthracene-2-amine (abbreviation: 2YGABPhA), N,N,9-triphenylanthracene-9-amine (abbreviation: DPhAPhA) or the like can be given, for example.

As the red fluorescent dopant, a tetracene derivative, a diamine derivative or the like can be given. Specifically, N,N,N′,N′-tetrakis(4-methylpheny)tetracene-5,11-diamine (abbreviation: p-mPhTD), 7,14-diphenyl-N,N,N′,N′-tetrakis(4-methylpheny)acenaphtho[1,2-a]fluoranthene-3,10-diamine (abbreviation: p-mPhAFD) orthe like can be given.

### <Phosphorescent Dopant>

As the phosphorescent dopant, a phosphorescent emitting heavy metal complex and a phosphorescent emitting rare earth metal complex can be given.

As the heavy metal complex, an iridium complex, an osmium complex, a platinum complex orthe like can be given. As the heavy metal complex, an ortho-metalated complex of a metal selected from iridium, osmium and platinum.

Examples of rare earth metal complexes include terbium complexes, europium complexes and the like. Specifically, tris(acetylacetonate)(monophenanthroline)terbium(III) (abbreviation: Tb(acac)₃(Phen)), tris(1,3-diphenyl-1,3-propandionate)(monophenanthroline)europium(III) (abbreviation: Eu(DBM)₃(Phen)), tris[1-(2-thenoyl)-3,3,3-trifluoroacetonate](monophenanthroline)europium(III) (abbreviation: Eu(TTA)(Phen)) or the like can be given. These rare earth metal complexes are preferable as phosphorescent dopants since rare earth metal ions emit light due to electronic transition between different multiplicity.

As the blue phosphorescent dopant, an iridium complex, an osmium complex, a platinum complex, or the like can be given, for example. Specifically, bis[2-(4′, 6′-difluoropheny)pyridinate-N,C2′]iridium(III) tetrakis(1-pyrazoly)borate (abbreviation: Flr6), bis[2-(4′, 6′-difluoropheny)pyridinate-N,C2′]iridium(III) picolinate (abbreviation: Flrpic), bis[2-(3′, 5′-bistrifluoromethylpheny)pyridinato-N,C2′]iridium(III) picolinate (abbreviation: Ir(CF3ppy)₂(pic)), bis[2-(4′, 6′-difluoropheny)pyridinato-N,C2′]iridium(III) acetylacetonate (abbreviation: Flracac) or the like can be given.

As the green phosphorescent dopant, an iridium complex or the like can be given, for example. Specifically, tris(2-phenylpyridinato-N,C2′) iridium(III) (abbreviation: Ir(ppy)₃), bis(2-phenylpyridinato-N,C2′)iridium(II) acetylacetonate (abbreviation: Ir(ppy)₂(acac)), bis(1,2-diphenyl-1H-benzimidazolato)iridium(III) acetylacetonate (abbreviation: Ir(pbi)₂(acac)), bis(benzo[h]quinolinato)iridium(III) acetylacetonate (abbreviation: Ir(bzq)₂(acac)) orthe like can be given.

As the red phosphorescent dopant, an iridium complex, a platinum complex, a terbium complex, a europium complex or the like can be given. Specifically, bis[2-(2′-benzo[4,5-a]thienyl)pyridinato-N,C3′]iridium(III) acetylacetonate (abbreviation: Ir(btp)₂(acac)), bis(1-phenylisoquinolinato-N,C2′)iridium(III) acetylacetonate (abbreviation: Ir(piq)₂(acac)), (acetylacetonato)bis[2,3-bis(4-fluoropheny)quinoxalinato]iridium(III) (abbreviation: Ir(Fdpq)₂(acac)), 2,3,7,8,12,13,17,18-octaethyl-21H,23H-porphyrin platinum(II) (abbreviation PtOEP) orthe like can be given.

### <Host Material>

As the host material, metal complexes such as aluminum complexes, beryllium complexes and zinc complexes; heterocyclic compounds such as indole derivatives, pyridine derivatives, pyrimidine derivatives, triazine derivatives, quinoline derivatives, isoquinoline derivatives, quinazoline derivatives, dibenzofuran derivatives, dibenzothiophene derivatives, oxadiazole derivatives, benzimidazole derivatives, phenanthroline derivatives; fused aromatic compounds such as a naphthalene derivative, a triphenylene derivative, a carbazole derivative, an anthracene derivative, a phenanthrene derivative, a pyrene derivative, a chrysene derivative, a naphthacene derivative, a fluoranthene derivative; and aromatic amine compound such as triarylamine derivatives and fused polycyclic aromatic amine derivatives can be given, for example. Plural types of host materials can be used in combination.

As specific examples of the metal complex, tris(8-quinolinolato)aluminum(III) (abbreviation: Alq), tris(4-methyl-8-quinolinolato)aluminum(III) (abbreviation: Almq3), bis(10-hydroxybenzo[h]quinolinato)beryllium(II) (abbreviation: BeBq2), bis(2-methyl-8-quinolinolato)(4-phenylphenolato)aluminum(III) (abbreviation: BAlq), bis(8-quinolinolato)zinc(II) (abbreviation: Znq), bis[2-(2-benzoxazoly)phenolato]zinc(II) (abbreviation: ZnPBO), bis[2-(2-benzothiazoly)phenolate]zinc(II) (abbreviation: ZnBTZ) orthe like can be given.

As specific examples of the heterocyclic compound, 2-(4-biphenyly)-5-(4-tert-butylphenyl)-1,3,4-oxadiazole (abbreviation: PBD),1,3-bis[5-(p-tert-butylphenyl)-1,3,4-oxadiazol-2-yl]benzene (abbreviation: OXD-7), 3-(4-biphenyly)-4-phenyl-5-(4-tert-butylpheny)-1,2,4-triazole (abbreviation: TAZ), 2,2′, 2″-(1,3,5-benzenetriy)tris(1-phenyl-1H-benzoimidazole)(abbreviation: TPBI), bathophenanthroline (abbreviation: BPhen), bathocuproine (abbreviation: BCP) or the like can be given.

As specific examples of the fused aromatic compound, 9-[4-(10-phenyl-9-anthry)phenyl]-9H-carbazole (abbreviation: CzPA), 3,6-diphenyl-9-[4-(10-phenyl-9-anthry)phenyl]-9H-carbazole (abbreviation: DPCzPA), 9,10-bis(3,5-diphenylpheny)anthracene (abbreviation: DPPA), 9,10-di(2-naphthy)anthracene (abbreviation: DNA), 2-tert-butyl-9,10-di(2-naphthy)anthracene (abbreviation: t-BuDNA), 9,9′-bianthryl (abbreviation: BANT), 9,9′-(stilbene-3,3′-diy)diphenanthrene (abbreviation: DPNS), 9,9′-(stilbene-4,4′-diy)diphenanthrene (abbreviation: DPNS2), 3,3′, 3-(benzene-1,3,5-triy)tripyrene (abbreviation: TPB3), 9,10-diphenylanthracene (abbreviation: DPAnth), 6,12-dimethoxy-5,11-diphenylcrysene or the like can be given.

As specific examples of the aromatic amine compound, N,N-diphenyl-9-[4-(10-phenyl-9-anthrylphenyl]-9H-carbazole-3-amine (abbreviation: CzA1PA), 4-(10-phenyl-9-anthry)triphenylamine (abbreviation: DPhPA), N,9-diphenyl-N-[4-(10-phenyl-9-anthry)phenyl]-9H-carbazole-3-amine (abbreviation: PCAPA), N,9-diphenylN-{4-[4-(10-phenyl-9-anthry)phenyl]phenyl}-9H-carbazole-3-amine (abbreviation: PCAPBA), N-(9,10-diphenyl-2-anthry)-N,9-diphenyl-9H-carbazole-3-amine (abbreviation: 2PCAPA), 4,4′-bis[N-(1-naphthy)-N-phenylamino]biphenyl (abbreviation: NPB ora-NPD), N,N′-bis(3-methylpheny)-N,N′-diphenyl-[1,1′-biphenyl]-4,4′-diamine (abbreviation: TPD), 4,4′-bis[N-(9,9-dimethylfluorene-2-yl)-N-phenylamino]biphenyl (abbreviation: DFLDPBi), 4,4′-bis[N-(spiro-9,9′-bifluorene-2-yl)-N-phenylamino]biphenyl (abbreviation: BSPB) or the like can be given.

As the fluorescent host, a compound having a higher singlet energy level than a fluorescent dopant is preferable. For example, a heterocyclic compound, a fused aromatic compound or the like can be given. As the fused aromatic compound, an anthracene derivative, a pyrene derivative, a chrysene derivative, a naphthacene derivative or the like are preferable.

As the phosphorescent host, a compound having a higher triplet energy level as compared with a phosphorescent dopant is preferable. For example, a metal complex, a heterocyclic compound, a fused aromatic compound or the like can be given. Among these, an indole derivative, a carbazole derivative, a pyridine derivative, a pyrimidine derivative, a triazine derivative, a quinoline derivative, an isoquinoline derivative, a quinazoline derivative, a dibenzofuran derivative, a dibenzothiophene derivative, a naphthalene derivative, a triphenylene derivative, a phenanthrene derivative, a fluoranthene derivative or the like can be given.

### (Electron-Transporting Layer)

An electron-transporting layer is a layer that comprises a substance having high electron-transporting property. As the substance having high electron-transporting property, a substance having an electron mobility of 10⁻⁶ cm₂/Vs or more is preferable. For example, a metal complex, an aromatic heterocyclic compound, an aromatic hydrocarbon compound, a polymeric compound or the like can be given.

As the metal complex, an aluminum complex, a beryllium complex, a zinc complex or the like can be given. Specifically, tris(8-quinolinolate)aluminum(III) (abbreviation: Alq), tris(4-methyl-8-quinolinolate)aluminum (abbreviation: Almq3), bis(10-hydroxybenzo[h]quinolinolate)beryllium (abbreviation: BeBq2), bis(2-methyl-8-quinollinolate)(4-phenylphenolate)aluminum(III) (abbreviation: BAlq), bis(8-quinolinolate)zinc(II) (abbreviation: Znq), bis[2-(2-benzoxazolyl)phenolato]zinc(II) (abbreviation: ZnPBO), bis[2-(2-benzothiazolyl phenolato]zinc(II) (abbreviation: ZnBTZ) or the like can be given, for example.

As the aromatic heterocyclic compound, imidazole derivatives such as benzimidazole derivatives, imidazopyridine derivatives and benzimidazophenanthridine derivatives; azine derivatives such as pyrimidine derivatives and triazine derivatives; a compound containing a nitrogen-containing six-membered ring structure such as quinoline derivatives, isoquinoline derivatives, and phenanthroline derivatives (including one having a phosphine oxide-based substituent on the heterocyclic ring) or the like can be given. Specifically, 2-(4-biphenylyl-5-(4-tert-butylphenyl-1,3,4-oxadiazole (abbreviation: PBD), 1,3-bis[5-(p-tert-butylphenyl)-1,3,4-oxadiazole-2-yl]benzene (abbreviation: OXD-7), 3-(4-tert-butylphenyl)-4-phenyl-5-(4-biphenylyl-1,2,4-triazole (abbreviation: TAZ), 3-(4-tert-butylpheny)-4-(4-ethylphenyl-5-(4-biphenylyl-1,2,4-triazole (abbreviation: p-EtTAZ), bathophenanthroline (abbreviation: BPhen), bathocuproine (abbreviation: BCP), 4,4′-bis(5-methylbenzoxazol-2-ylstilbene (abbreviation: BzOs) or the like can be given.

As the aromatic hydrocarbon compound, an anthracene derivative, a fluoranthene derivative orthe like can be given, for example.

As specific examples of the polymeric compound, poly[(9,9-dihexylfluorene-2,7-diyl-co-(pyridine-3,5-diyl)](abbreviation: PF-Py), poly[(9,9-dioctylfluorene-2,7-diyl)-co-(2,2′-bipyridine-6,6′-diyl] (abbreviation: PF-BPy) orthe like can be given.

As long as it is a compound having a higher electron-transporting property as compared with hole-transporting property, such a compound may be used in the electron-transporting layer in addition to the substances mentioned above.

The electron-transporting layer may be a single layer, or a stacked layer of two or more layers. In this case, it is preferable to arrange a layerthat includes a substance having a larger energy gap, among substances having a high electron-transporting capability, on the side nearer to the emitting layer. For example, as shown in FIG. 2, a configuration including the first electron-transporting layer 7a on the anode side and the second electron-transporting layer 7b on the cathode side may be employed.

The electron-transporting layer may contain a metal such as an alkali metal, magnesium, an alkaline earth metal, or an alloy containing two or more of these metals; a metal compound such as an alkali metal compound such as 8-quinolinolato lithium (Liq), or an alkaline earth metal compound. When a metal such as an alkali metal, magnesium, an alkaline earth metal, or an alloy containing two or more of these metals is contained in the electron-transporting layer, the content of the metal is not particularly limited, but is preferably from 0.1 to 50 mass %, more preferably from 0.1 to 20 mass %, further preferably from 1 to 10 mass %.

When a metal compound such as an alkali metal compound or an alkaline earth metal compound is contained in the electron-transporting layer, the content of the metal compound is preferably 1 to 99 mass %, more preferably from 10 to 90 mass %. When the electron-transporting layer is formed of plural layers, a layer on the emitting layer side can be formed only from these metal compounds.

### (Electron-Injecting Layer)

The electron-injecting layer is a layer that includes a substance that has a high electron-injecting capability, and has the function of efficiently injecting electrons from a cathode to an emitting layer. Examples of the substance that has a high electron-injecting capability include an alkali metal, magnesium, an alkaline earth metal, and a compound thereof. Specific examples thereof include lithium, cesium, calcium, lithium fluoride, cesium fluoride, calcium fluoride, lithium oxide, and the like. In addition, an electron-transporting substance having electron-transporting property in which is incorporated with an alkali metal, magnesium, an alkaline earth metal, or a compound thereof is incorporated, for example, Alq incorporated with magnesium, may also be used.

Alternatively, a composite material that includes an organic compound and a donor compound may also be used in the electron-injecting layer. Such a composite material is excellent in the electron-injecting capability and the electron-transporting capability since the organic compound receives electrons from the donor compound.

The organic compound is preferably a material excellent in transporting capability of the received electrons, and specifically, for example, a metal complex, an aromatic heterocyclic compound, or the like, which is a substance that has a high electron-transporting capability mentioned above, can be used.

Any material capable of donating its electron to the organic compound can be used as the donor compound. Examples thereof include an alkali metal, magnesium, an alkaline earth metal, and a rare earth metal. Specific examples thereof include lithium, cesium, magnesium, calcium, erbium, and ytterbium. Further, an alkali metal oxide and an alkaline earth metal oxide are preferred, and examples thereof include lithium oxide, calcium oxide, and barium oxide. In addition, a Lewis base such as magnesium oxide can be used. Moreover, an organic compound such as tetrathiafulvalene (abbreviation: TTF) can also be used.

### (Cathode)

For the cathode, a metal, an alloy, an electrically conductive compound, and a mixture thereof, each having a small work function (specifically, a work function of 3.8 eV or less) are preferably used. Specific examples of the material for such a cathode include an alkali metal such as lithium and cesium; magnesium; an alkaline earth metal such as calcium, and strontium; an alloy containing these metals (for example, magnesium-silver, aluminum-lithium); a rare earth metal such as europium and ytterbium; and an alloy containing a rare earth metal.

The cathode is usually formed by a vacuum vapor deposition or a sputtering method. Further, in the case of using a silver paste or the like, a coating method, an inkjet method, or the like can be employed.

Moreover, when the electron-injecting layer is provided, various electrically conductive materials such as aluminum, silver, ITO, graphene, indium oxide-tin oxide containing silicon or silicon oxide, selected independently from the work function, can be used to form a cathode. These electrically conductive materials are made into films using a sputtering method, an inkjet method, a spin coating method, or the like.

### (Insulating Layer)

In the organic EL device, pixel defects based on leakage or a short circuit are easily generated since an electric field is applied to a thin film. In order to prevent this, it is preferred to insert an insulating thin layer between a pair of electrodes.

Examples of materials used in the insulating layer include aluminum oxide, lithium fluoride, lithium oxide, cesium fluoride, cesium oxide, magnesium oxide, magnesium fluoride, calcium oxide, calcium fluoride, aluminum nitride, titanium oxide, silicon oxide, germanium oxide, silicon nitride, boron nitride, molybdenum oxide, ruthenium oxide, and vanadium oxide. A mixture thereof may be used in the insulating layer, and a laminate of a plurality of layers that include these materials can be also used for the insulating layer.

### (Spacing Layer)

The spacing layer is a layer provided between the fluorescent emitting layer and the phosphorescent emitting layer when the fluorescent emitting layer and the phosphorescent emitting layer are stacked in order to prevent diffusion of excitons generated in the phosphorescent emitting layer to the fluorescent emitting layer or in order to adjust the carrier balance. Further, the spacing layer can be provided between the plural phosphorescent emitting layers.

Since the spacing layer is provided between the emitting layers, the material used for the spacing layer is preferably a material having both electron-transporting capability and hole-transporting capability. In order to prevent diffusion of the triplet energy in adjacent phosphorescent emitting layers, it is preferred that the spacing layer have a triplet energy of 2.6 eV or more.

As the material used for the spacing layer, the same materials as those used in the above-mentioned hole-transporting layer can be given.

### (Electron-Blocking Layer, Hole-Blocking Layer, Exciton-Blocking Layer)

An electron-blocking layer, a hole-blocking layer, an exciton (triplet)-blocking layer, and the like may be provided in adjacent to the emitting layer.

The electron-blocking layer has a function of preventing leakage of electrons from the emitting layer to the hole-transporting layer. The hole-blocking layer has a function of preventing leakage of holes from the emitting layer to the electron-transporting layer. The exciton-blocking layer has a function of preventing diffusion of excitons generated in the emitting layer to the adjacent layers and confining the excitons within the emitting layer.

### (Method for Forming a Layer)

The method forforming each layer of the organic EL device of the invention is not particularly limited unless otherwise specified. A known film-forming method such as a dry film-forming method, a wet film-forming method or the like can be used. Specific examples of the dry film-forming method include a vacuum deposition method, a sputtering method, a plasma method, an ion plating method, and the like. Specific examples of the wet film-forming method include various coating methods such as a spin coating method, a dipping method, a flow coating method, an inkjet method, and the like.

### (Film Thickness)

The film thickness of each layer of the organic EL device of the invention is not particularly limited unless otherwise specified. If the film thickness is too small, defects such as pinholes are likely to occur to make it difficult to obtain an enough luminance. If the film thickness is too large, a high driving voltage is required to be applied, leading to a lowering in efficiency. In this respect, the film thickness is preferably 5 nm to 10 μm, and more preferably 10 nm to 0.2 μm.

### [Electronic Appliance]

The electronic appliance according to an aspect of the invention includes the above-described organic EL device according to an aspect of the invention. Examples of the electronic appliance include display parts such as an organic EL panel module; display devices of television sets, mobile phones, smart phones, and personal computer, and the like; and emitting devices of a lighting device and a vehicle lighting device.

### EXAMPLES

Next, the invention will be explained in more detail in accordance with the following synthesis examples, examples, and comparative examples, which should not be construed as limiting the scope of the invention.

### Example 1

### Synthesis of Compound 1A

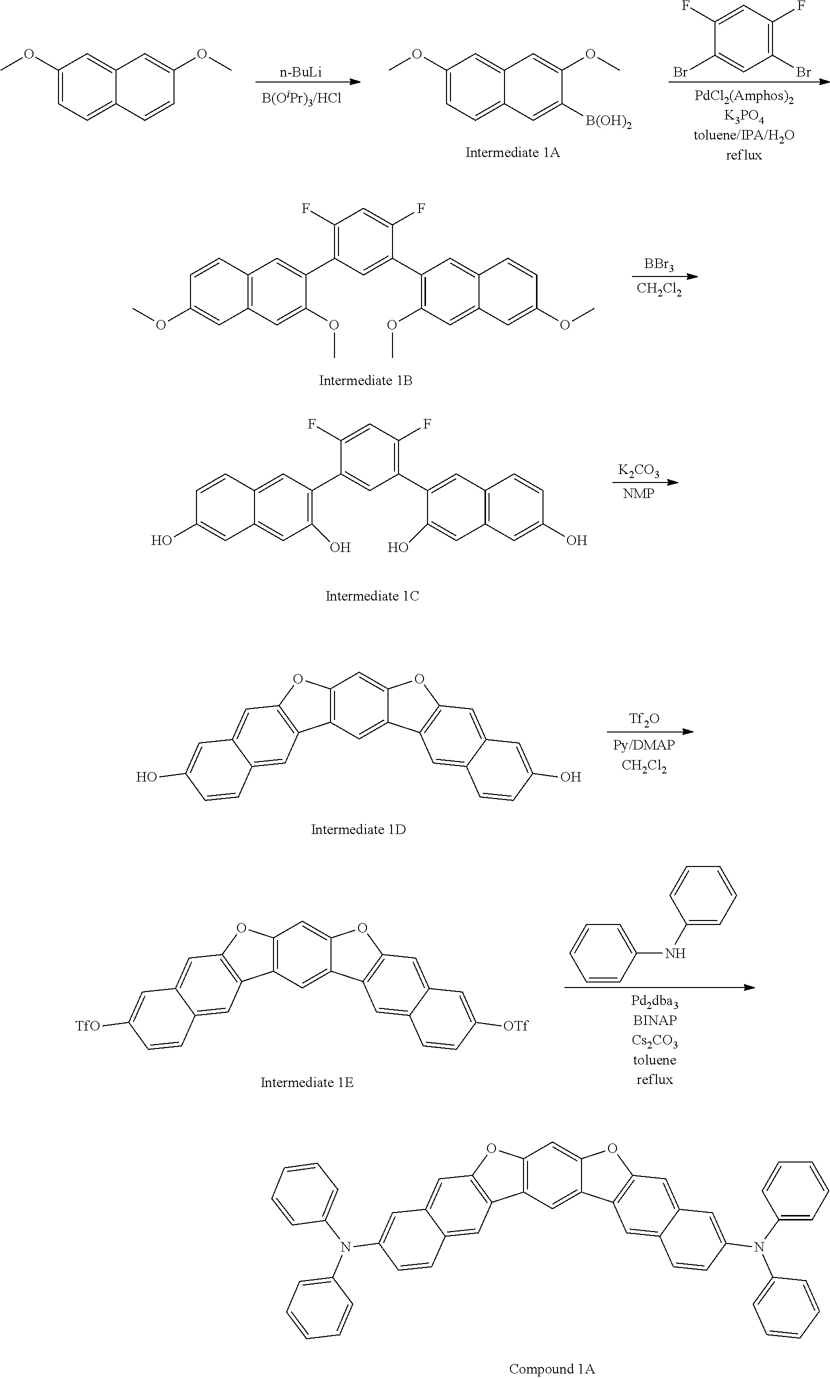

### (1-1) Synthesis of Intermediate 1A

Under an argon atmosphere, 350 mL solution of 2,7-dimethoxynaphthalene (10 g) in tetrahydrofuran was cooled to 0° C., and 1.55 M n-butyllithium hexane (34.3 mL) was added dropwise thereto over 20 minutes, followed by stirring at 0° C. for another 1 hour. The reaction solution was then cooled to −78° C. and triisopropylborate (17.1 mL) was added dropwise thereto. After stirring for 2 hours while returning to room temperature, 50 mL of 4 N hydrochloric acid was added. The solvent was removed from the reaction solution, and precipitated crystals were washed with water and hexane to obtain Intermediate 1A (10.9 g, 88% yield).

### (1-2) Synthesis of Intermediate 1B

Under an argon atmosphere, a mixture of Intermediate 1A(1.9 g) obtained in (1-1), 1,5-dibromo-2,4-difluorobenzene (0.13 g), bis[4-[bis(tert-butylphosphino]-N,N-dimethylbenzenaminoamino]dichloropalladium (PdCl₂(Amphos)₂) (0.23 g), potassium phosphate (1.7 g), 120 mL of toluene, 40 mL of isopropyl alcohol, and 20 mL of water was refluxed for 18 hours. The obtained reaction solution was cooled to room temperature, and extracted with toluene. The organic phase was washed with water, and dried over anhydrous sodium sulfate, and the solvent was distilled off under reduced pressure. The obtained residue was purified by silica gel column chromatography to obtain Intermediate 1B (1.5 g, 83% yield).

### (1-3) Synthesis of Intermediate 1C

Under an argon atmosphere, 10 mL solution of Intermediate 1B (1.4 g) obtained in (1-2) in methylene chloride was cooled to −10° C. (internal temperature), and 1 M solution of boron tribromide in methylene chloride (12.1 mL) was added dropwise thereto. The reaction was allowed to stand at −10° C. for 1 hour and stirred for 2 hours while returning to room temperature. The obtained solution was poured into ice water, extracted with ethyl acetate, and dried over anhydrous sodium sulfate. The solvent was distilled off under reduced pressure to obtain Intermediate 1C (1.3 g, 100% yield).

### (1-4) Synthesis of Intermediate 1D

Under an argon atmosphere, a mixture of Intermediate 1C (1.3 g) obtained in (1-3), potassium carbonate (1.7 g), and 150 mL of N-methylpyrrolidone was stirred at 150° C. for 5 hours. The obtained reaction solution was cooled to room temperature, an insoluble was removed by filtration, and 200 mL of a saturated aqueous ammonium chloride solution and 200 mL of ethyl acetate were added to the reaction solution. The organic phase was further washed with water, and dried over anhydrous sodium sulfate. The solvent was distilled off under reduced pressure to obtain Intermediate 1D (1.1 g, 95% yield). The molecular weight of the target compound was 390.39, and the mass spectrum of the resulting compound was analyzed as m/z (ratio of mass to charge)=390, thereby identified as the target compound.

### (1-5) Synthesis of Intermediate 1E

Under an argon atmosphere, a mixture of Intermediate 1D (2.0 g) obtained in (1-4), N,N-dimethyl-4-aminopyridine (0.63 g), pyridine (20 mL) and methylene chloride (100 mL) was stirred under ice cooling, and then, trifluoromethanesulfonic anhydride (2.2 mL) was added dropwise thereto. After the reaction was carried out for 15 minutes under ice cooling, the reaction was carried out for 7 hours while returning to room temperature.

Methanol and water were added to the obtained reaction solution under ice cooling, followed by stirring, and crystals precipitated after concentration were separated by filtration. The crystals were washed with water, methanol and ethyl acetate to obtain Intermediate 1E (2.4 g, 71% yield). The molecular weight of the target compound was 654.50, and the mass spectrum of the resulting compound was analyzed as m/z (ratio of mass to charge)=654, thereby identified as the target compound.

### (1-6) Synthesis of Compound 1A

Under an argon atmosphere, a mixture of Intermediate 1E (50 mg) obtained in (1-5), 39 mg (0.23 mmol) of diphenylamine, palladium acetate (Pd(OAc)₂) (0.86 mg), (±)-2,2′-bis(diphenylphosphino)-1,1′-binaphthyl (BINAP) (4.8 mg), cesium carbonate (100 mg), and toluene (5 mL) was refluxed for 20 hours. The obtained reaction solution was cooled to room temperature, and extracted with toluene. The organic phase was washed with water, and dried over anhydrous sodium sulfate. The solvent was distilled off under reduced pressure. The obtained residue was purified by silica gel column chromatography to obtain Compound 1 (37 mg, 69% yield). The molecular weight of Compound 1A was 692.82, and the mass spectrum of the resulting compound was analyzed as m/z (ratio of mass to charge)=692, thereby identified as the target compound.

Compound within the scope of the invention can be synthesized by using known alternative reactions or raw materials suitable for the target compound in accordance with the above reaction.

### (Preparation of Toluene Solution)

The resulting Compound 1A was dissolved in toluene to a concentration of 5 μmol/L to prepare a toluene solution of Compound 1.

### (Measurement of Fluorescent Quantum Yield (PLQY))

PLQY of the obtained toluene solution of Compound 1A was measured using an absolute PL (photoluminescence) quantum yield measuring device Quantaurus-QY (manufactured by Hamamatsu Photonics K.K.). PLQY of Compound 1 was 80%.

### (Measurements of Fluorescence Emission Peak-Wavelength (FL-Peak))

The obtained toluene solution of Compound 1A was measured using a fluorescent spectrofluorometer F-7000 (manufactured by Hitachi High-Tech Science Corporation) of a fluorescence spectrophotometer, and a fluorescence emission peak wavelength was observed at 427 nm when the toluene solution was excited at 360 nm.

For each compound obtained in Examples 2 to 24, toluene solution was prepared and evaluated in the same manner as in Example 1. The results are shown in Table 1.

### Example 2

### Synthesis of Compound 1B

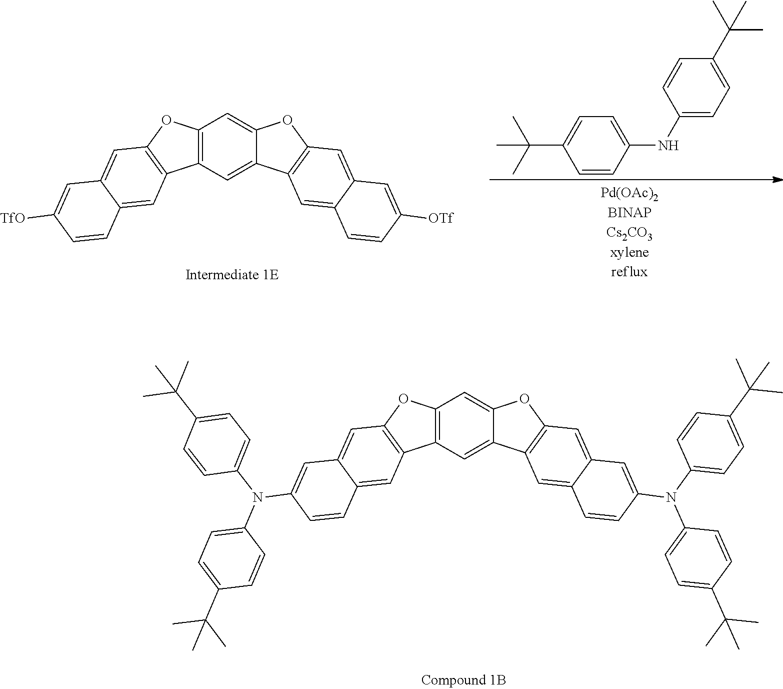 [SMILES: O=S(=O)(Oc1ccc2cc3c(cc2c1)oc1cc2oc4cc5cc(OS(=O)(=O)C(F)(F)F)ccc5cc4c2cc13)C(F)(F)F ; CC(C)(C)c1ccc(Nc2ccc(C(C)(C)C)cc2)cc1 ; CC(C)(C)c1ccc(N(c2ccc(C(C)(C)C)cc2)c2ccc3cc4c(cc3c2)oc2cc3oc5cc6cc(N(c7ccc(C(C)(C)C)cc7)c7ccc(C(C)(C)C)cc7)ccc6cc5c3cc24)cc1]

Under an argon atmosphere, a mixture of Intermediate 1E (0.7 g), bis-(4-(tert-butyl)phenyl)amine (1.8 g), palladium acetate (Pd(OAc)₂) (0.024 g), BINAP (0.133 g), cesium carbonate (2.79 g), and 100 mL of toluene was refluxed for 6 hours. The obtained reaction solution was cooled to room temperature, and extracted with toluene. The organic phase was washed with water, and dried over anhydrous sodium sulfate. The solvent was distilled off under reduced pressure. The resulting residue was purified by silica gel column chromatography to obtain Compound 1B (0.2 g, 20% yield). The molecular weight of Compound 1B was 917.25, and the mass spectrum of the resulting compound was analyzed as m/z (ratio of mass to charge)=917, thereby identified as the target compound.

### Example 3

### Synthesis of Compound 1C

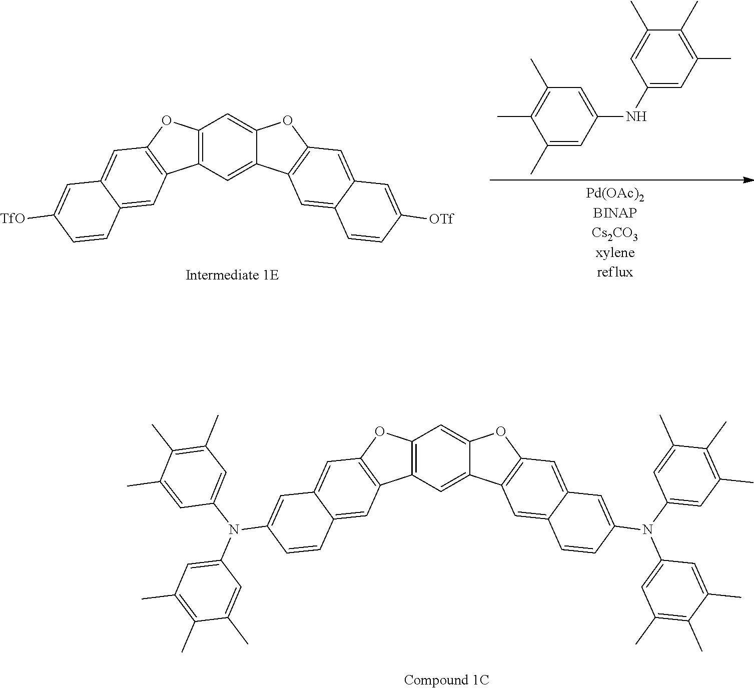

Under an argon atmosphere, a mixture of Intermediate 1E (30 mg), bis(3,4,5-trimethylphenylamine (69.7 mg), palladium acetate (Pd(OAc)₂) (1.03 mg), BINAP (5.71 mg), cesium carbonate (119 mg), and 5 mL of toluene was refluxed for 6 hours. The obtained reaction solution was cooled to room temperature, and extracted with toluene. The organic phase was washed with water, and dried over anhydrous sodium sulfate. The solvent was distilled off under reduced pressure. The resulting residue was purified by silica gel column chromatography to obtain Compound 1C (10 mg, 25% yield). The molecular weight of Compound 1C was 861.14, and the mass spectrum of the resulting compound was analyzed as m/z (ratio of mass to charge)=861, thereby identified as the target compound.

### Example 4

### Synthesis of Compound 2A

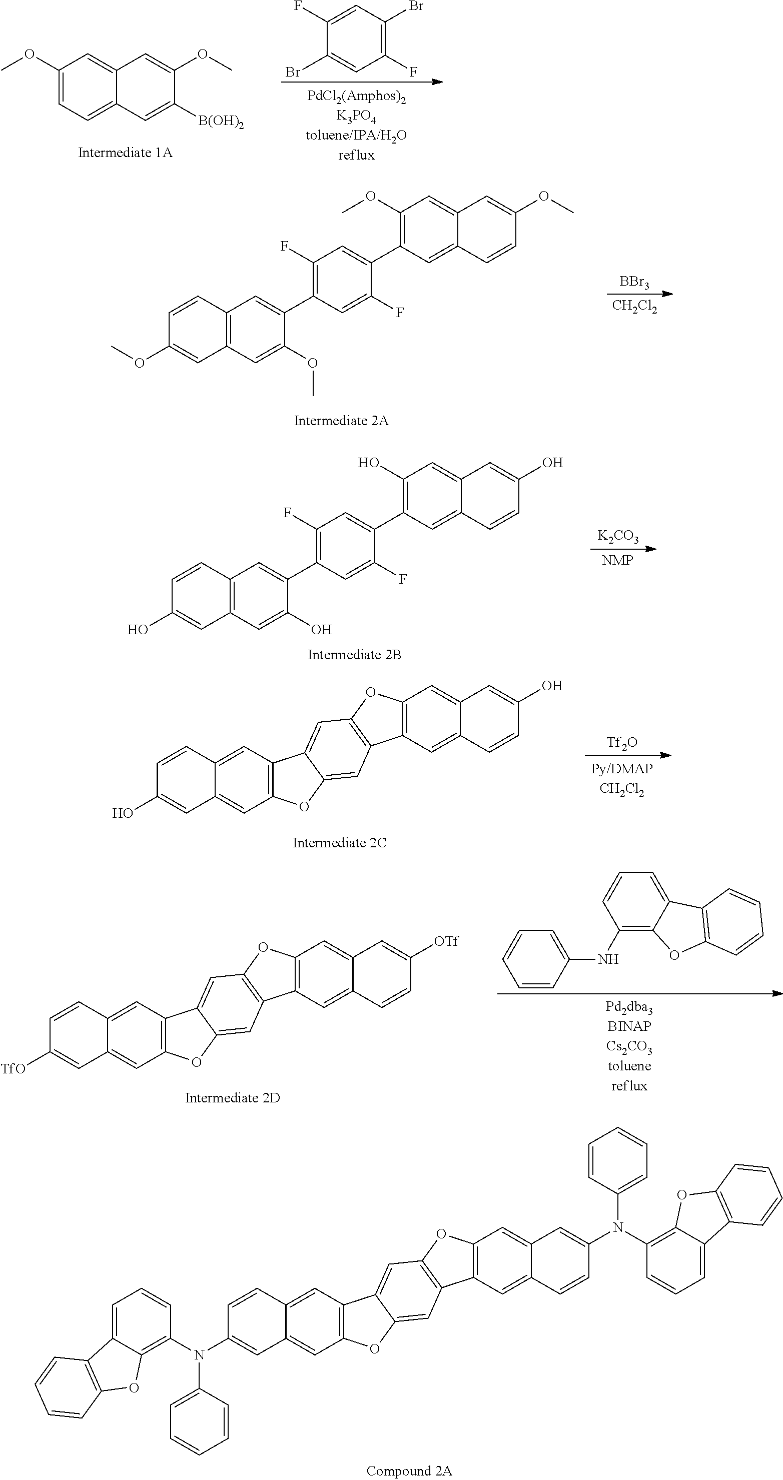

### (4-1) Synthesis of Intermediate 2A

In the synthesis of Intermediate 1B, 1,4-dibromo-2,5-difluorobenzene (1.6 g) was used instead of 1,5-dibromo-2,4-difluorobenzene to obtain Intermediate 2A (1.85 g, 65% yield).

### (4-2) Synthesis of Intermediate 2B

In the synthesis of Intermediate 1C, Intermediate 2A (2.5 g) was used instead of Intermediate 1B to obtain Intermediate 2B (2.2 g, 100% yield).

### (4-3) Synthesis of Intermediate 2C

In the synthesis of Intermediate 1D, Intermediate 2B (2.2 g) was used instead of Intermediate 1C to obtain Intermediate 2C (1.7 g, 85% yield). The molecular weight of the target compound was 390.39, and the mass spectrum of the resulting compound was analyzed as m/z (ratio of mass to charge)=390, thereby identified as the target compound.

### (4-4) Synthesis of Intermediate 2D

In the synthesis of Intermediate 1E, Intermediate 2C (1.7 g) was used instead of Intermediate 1D to obtain Intermediate 2D (2.2 g, 77% yield). The molecular weight of the target compound was 654.50, and the mass spectrum of the resulting compound was analyzed as m/z (ratio of mass to charge)=654, thereby identified as the target compound.

### (4-5) Synthesis of Compound 2A

In the synthesis of Compound 1A, Intermediate 2D (30 mg) was used instead of Intermediate 1E to obtain Compound 2A(2 mg, 5% yield). The molecular weight of Compound 2A was 872.98, and the mass spectrum of the resulting compound was analyzed as m/z (ratio of mass to charge)=872, thereby identified as the target compound.

### Example 5

### Synthesis of Compound 3A

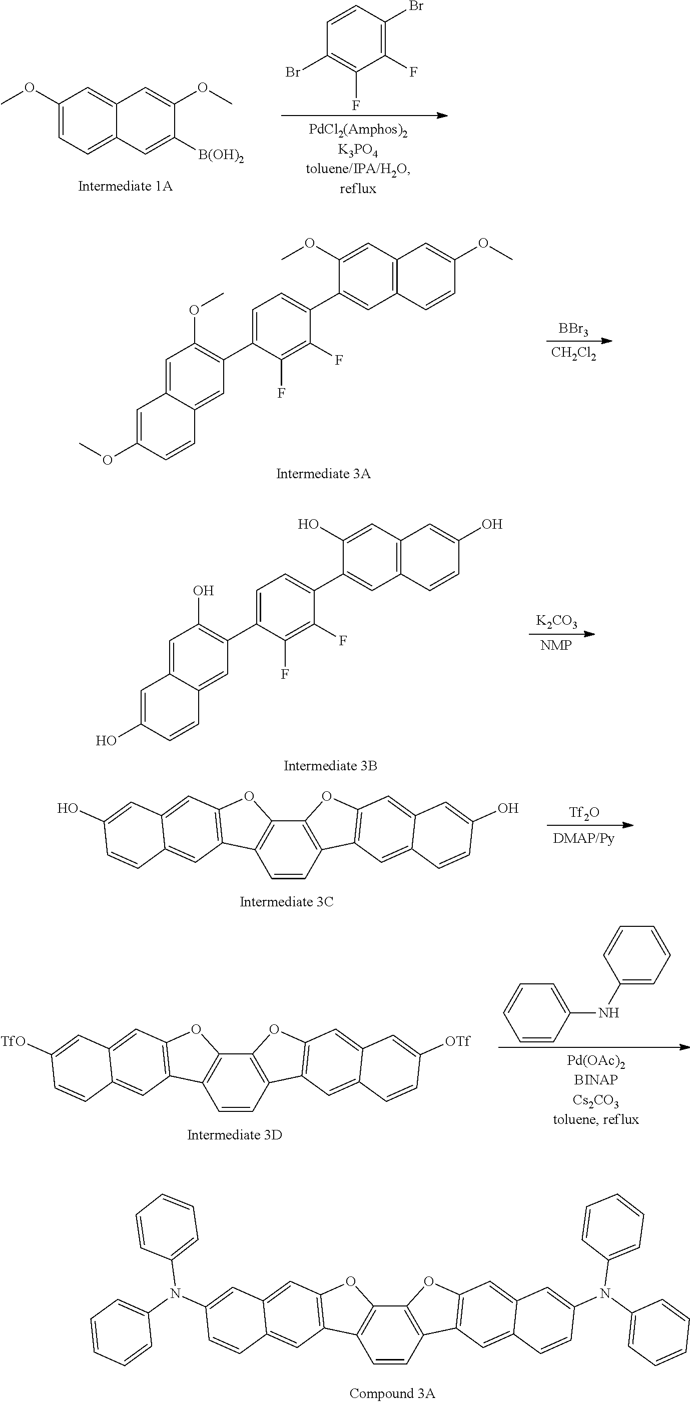

### (5-1) Synthesis of Intermediate 3A

In the synthesis of the Intermediate 1B, 1,4-dibromo-2,3-difluorobenzene (3.7 g) was used instead of 1,5-dibromo-2,4-difluorobenzene to obtain Intermediate 3A (1.8 g, 50% yield).

### (5-2) Synthesis of Intermediate 3B

In the synthesis of Intermediate 1C, Intermediate 3A (1.8 g) was used instead of Intermediate 1B to obtain Intermediate 3B (1.1 g, 69% yield).

### (5-3) Synthesis of Intermediate 3C

In the synthesis of Intermediate 1D, Intermediate 3B (1.1 g) was used instead of Intermediate 1C to obtain Intermediate 3C (0.6 g, 60% yield). The molecular weight of the target compound was 390.39, and the mass spectrum of the resulting compound was analyzed as m/z (ratio of mass to charge)=390, thereby identified as the target compound.

### (5-4) Synthesis of Intermediate 3D

In the synthesis of Intermediate 1E, Intermediate 3C (0.6 g) was used instead of Intermediate 1D to obtain Intermediate 3D (0.5 g, 50% yield). The molecular weight of the target compound was 654.50, and the mass spectrum of the resulting compound was analyzed as m/z (ratio of mass to charge)=654, thereby identified as the target compound.

### (5-5) Synthesis of Compound 3A

In the synthesize of Compound 1A, Intermediate 3D (0.05 g) was used instead of Intermediate 1E to obtain Compound 3A(12.3 mg, 23% yield). The molecular weight of Compound 3A was 692.82, and the mass spectrum of the resulting compound was analyzed as m/z (ratio of mass to charge)=692, thereby identified as the target compound.

### Example 6

### Synthesis of Compound 4A

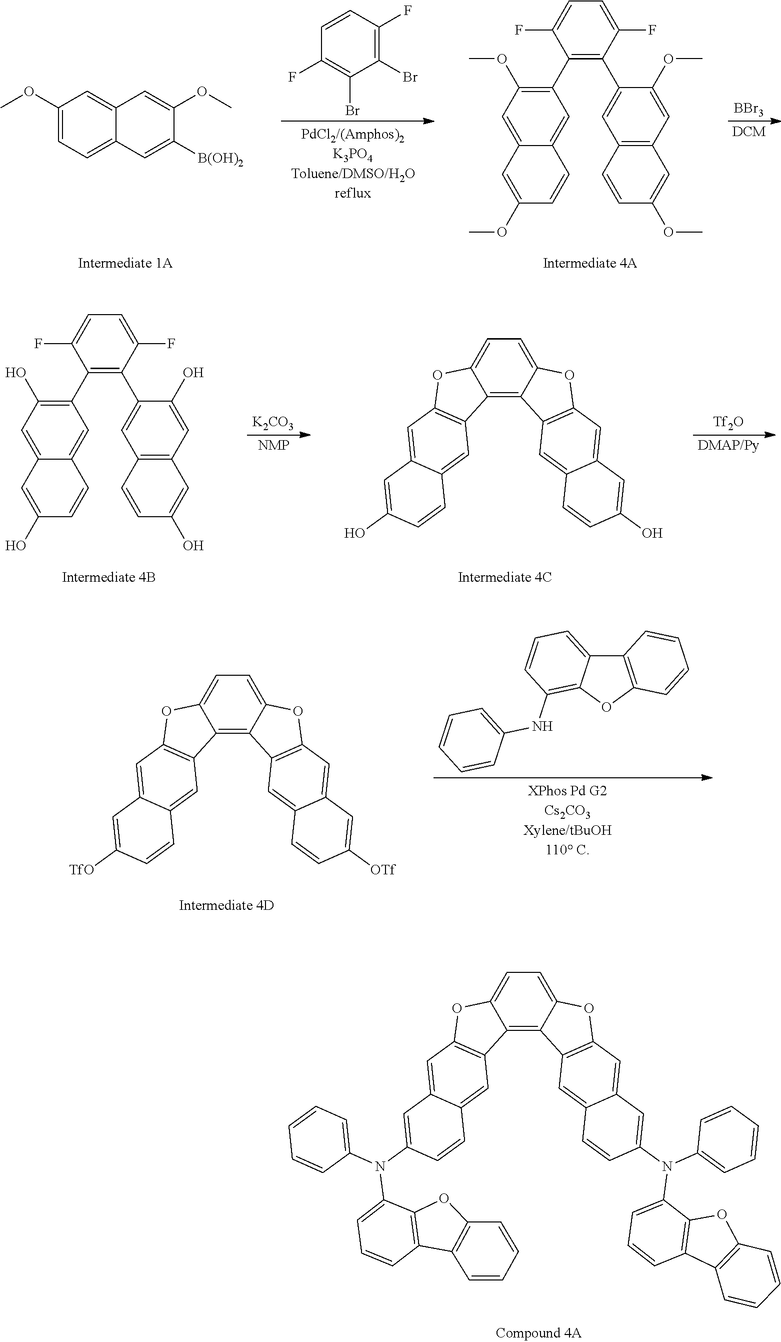

### (6-1) Synthesis of Intermediate 4A

Under an argon atmosphere, a mixture of Intermediate 1A (10.2 g), 2,3-dibromo-1,4-difluorobenzene (3.0 g), (PdCl₂(Amphos)₂ (0.39 g), potassium phosphate (9.3 g), toluene (72 mL), dimethyl sulfoxide (DMSO) (24 mL), and water (12 mL) was refluxed for 18 hours. The obtained reaction solution was cooled to room temperature, and extracted with toluene. The organic phase was washed with water, and dried over anhydrous sodium sulfate. The solvent was distilled off under reduced pressure. The obtained residue was purified by silica gel column chromatography to obtain Intermediate 4A (2.4 g, 46% yield). The molecular weight of the target compound was 486.51, and the mass spectrum of the resulting compound was analyzed as m/z (ratio of mass to charge)=486, thereby identified as the target compound.

### (6-2) Synthesis of Intermediate 4B

In the synthesize of Intermediate 1C, Intermediate 4A (1.6 g) was used instead of Intermediate 1B to obtain Intermediate 4B (1.4 g, 96% yield). The molecular weight of the target compound was 430.41, and the mass spectrum of the resulting compound was analyzed as m/z (ratio of mass to charge)=430, thereby identified as the target compound.

### (6-3) Synthesis of Intermediate 4C

In the synthesize of Intermediate 1D, Intermediate 4B (2.3 g) was used instead of Intermediate 1C to obtain Intermediate 4C (1.2 g, 59% yield). The molecular weight of the target compound was 390.39, and the mass spectrum of the resulting compound was analyzed as m/z (ratio of mass to charge)=390, thereby identified as the target compound.

### (6-4) Synthesis of Intermediate 4D

In the synthesize of Intermediate 1E, Intermediate 4C (1.2 g) was used instead of Intermediate 1D to obtain Intermediate 4D (0.7 g, 36% yield). The molecular weight of the target compound was 654.50, and the mass spectrum of the resulting compound was analyzed as m/z (ratio of mass to charge)=654, thereby identified as the target compound.

### (6-5) Synthesis of Compound 4A

Under an argon atmosphere, a mixture of Intermediate 4D (250 mg), N-phenyldibenzofuran-4-amine (248 mg), XPhos Pd G2 (manufactured by Sigma-Aldrich Co. LLC.) (9 mg), cesium carbonate (498 mg), xylenes (25 mL), tert-butanol (tBuOH) (5 mL) was stirred at 110° C. for 4 hours. The obtained reaction solution was cooled to room temperature, and extracted with toluene. The organic phase was washed with water, and dried over anhydrous magnesium sulfate. The solvent was distilled off under reduced pressure. The resulting residue was purified by silica gel column chromatography to obtain Compound 4A (67 mg, 20% yield). The molecular weight of Compound 4A was 872.980, and the mass spectrum of the resulting compound was analyzed as m/z (ratio of mass to charge)=872, thereby identified as the target compound.

### Example 7

### Synthesis of Compound 1D

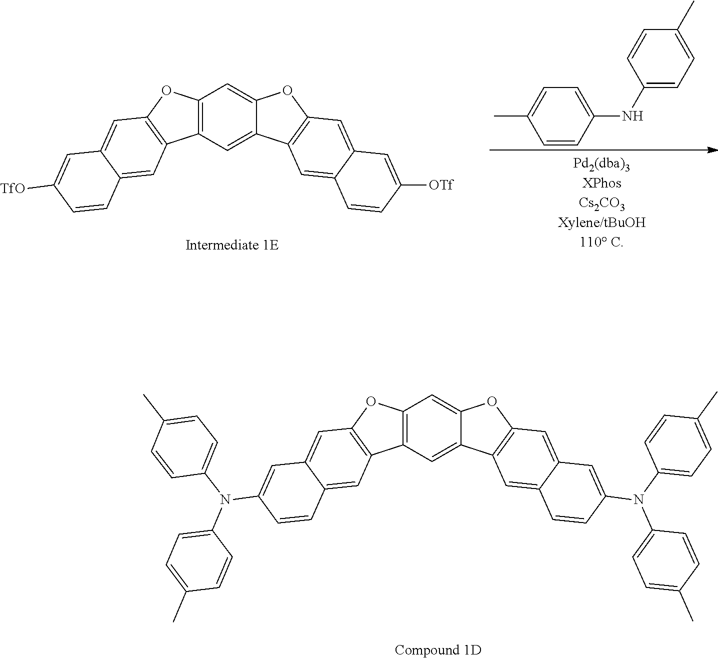

### Synthesis of Compound 1D

Under an argon atmosphere, a mixture of Intermediate 1E (1.0 g), di-p-tolylamine (0.754 g), tris(dibenzylideneacetone)dipalladium (0) (Pd₂(dba)₃) (0.028 g), XPhos (0.058 g), cesium carbonate (1.99 g), xylene (100 mL), and tert-butanol (20 mL) was stirred at 110° C. for 5 hours. The obtained reaction solution was cooled to room temperature, and extracted with toluene. The organic phase was washed with water, and dried over anhydrous sodium sulfate. The solvent was distilled off under reduced pressure. The obtained residue was purified by silica gel column chromatography to obtain Compound 1D (0.54 g, 47% yield). The molecular weight of Compound 1D was 748.926, and the mass spectrum of the resulting compound was analyzed as m/z (ratio of mass to charge)=748, thereby identified as the target compound.

### Example 8

### Synthesis of Compound 1E

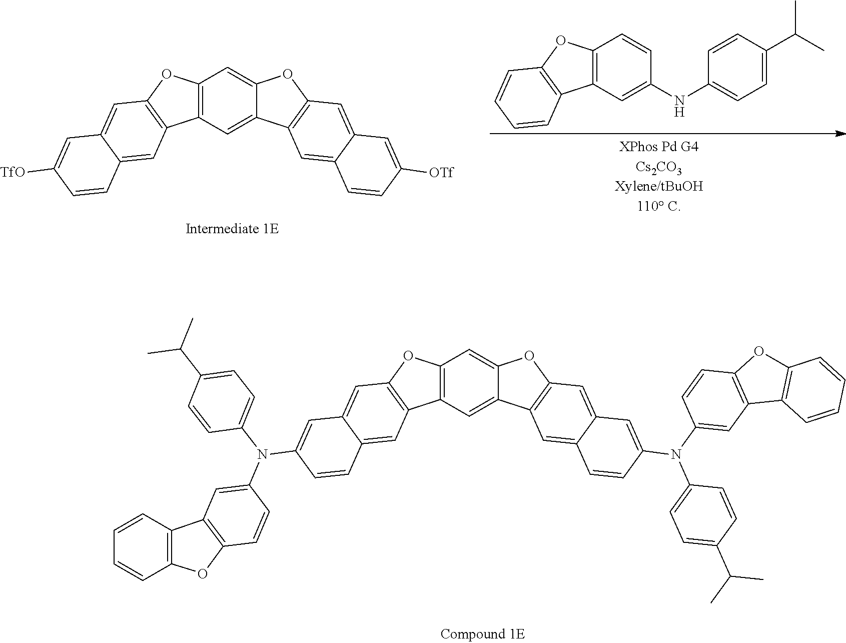 [SMILES: O=S(=O)(Oc1ccc2cc3c(cc2c1)oc1cc2oc4cc5cc(OS(=O)(=O)C(F)(F)F)ccc5cc4c2cc13)C(F)(F)F ; CC(C)c1ccc(Nc2ccc3oc4ccccc4c3c2)cc1 ; CC(C)c1ccc(N(c2ccc3cc4c(cc3c2)oc2cc3oc5cc6cc(N(c7ccc(C(C)C)cc7)c7ccc8oc9ccccc9c8c7)ccc6cc5c3cc24)c2ccc3oc4ccccc4c3c2)cc1]

Under an argon atmosphere, a mixture of Intermediate 1E (1.0 g), N-[4-(propane-2-ylphenyl]dibenzo[b,d]furan-2-amine (1.1 g), XPhos Pd G4 (manufactured by Sigma-Aldrich Co. LLC.) (0.039 g), cesium carbonate (1.9 g), xylenes (100 mL), tert-butanol (20 mL) was stirred at 110° C. for 4 hours. The obtained reaction solution was cooled to room temperature, and extracted with toluene. The organic phase was washed with water, and dried over anhydrous magnesium sulfate. The solvent was distilled off under reduced pressure. The obtained residue was purified by silica gel column chromatography and recrystallization to obtain Compound 1E (1.27 g, 87% yield). The molecular weight of Compound 1E was 957.142, and the mass spectrum of the resulting compound was analyzed as m/z (ratio of mass to charge)=957, thereby identified as the target compound.

### Example 9

### Synthesis of Compound 5A

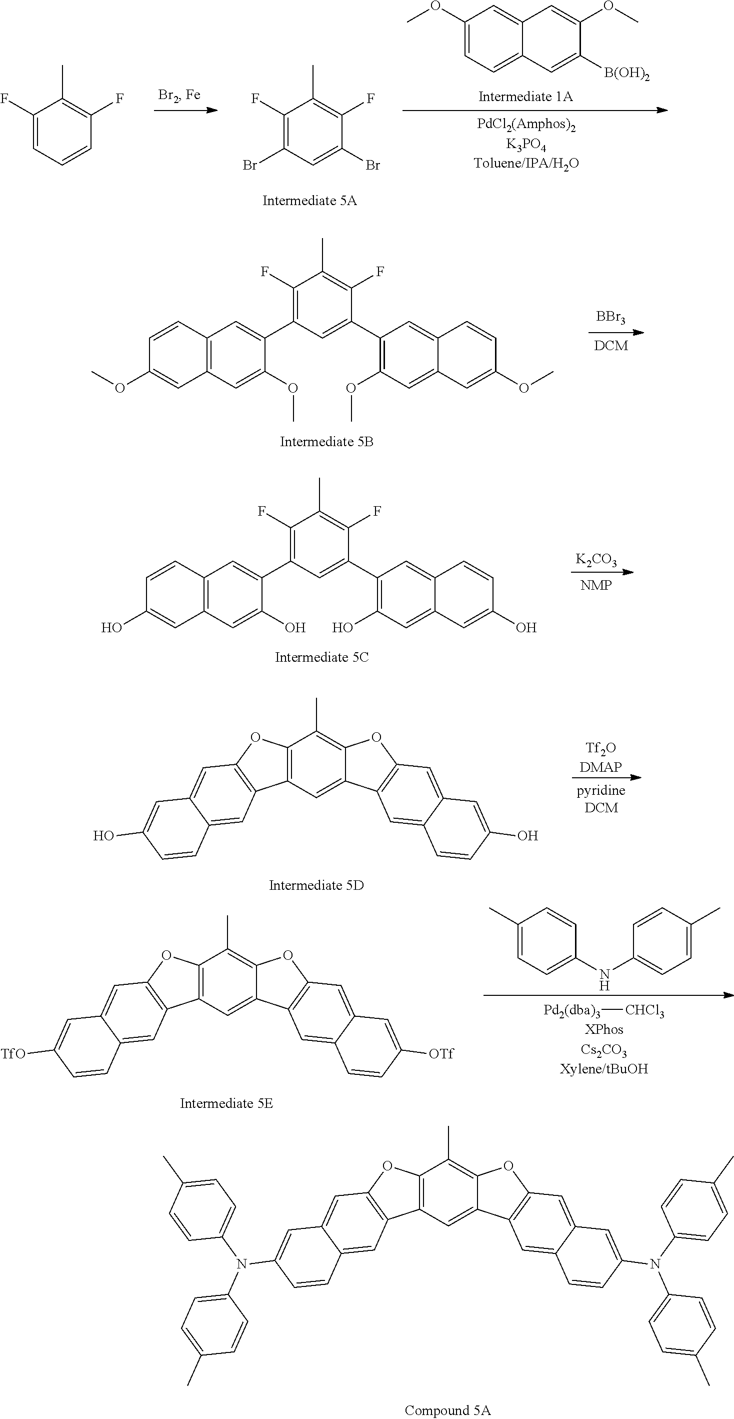

### (9-1) Synthesis of Intermediate 5A

Under an argon atmosphere, 1,3-difluoro-2-methylbenzene (9.9 g) was cooled to0° C. in ice bath, followed by the addition of iron powder (0.51 g). Then, bromine (29.6 g) was slowly added dropwise thereto and stirred at room temperature for 10 hours. After hexanes was added to the obtained reaction solution and extracted, the organic phase was washed with an aqueous solution of sodium bisulfite, and dried over anhydrous sodium sulfate. The solvent was distilled off under reduced pressure to obtain Intermediate 5A (20 g, yield: 93%). The molecular weight of the target compound was 285.91, and the mass spectrum of the resulting compound was analyzed as m/z(ratio of mass to charge)=285, thereby identified as the target compound.

### (9-2) Synthesis of Intermediate 5B

Under an argon atmosphere, a mixture of Intermediate 5A (3.6 g), Intermediate A (6.4 g), PdCl₂(Amphos)₂ (0.44 g), potassium phosphate (5.8 g), toluene (168 mL), isopropyl alcohol (56 mL), and water (28 mL) was stirred at 80° C. for 6 hours. The obtained reaction solution was cooled to room temperature, and extracted with toluene. The organic phase was washed with water, and dried over anhydrous sodium sulfate. The solvent was distilled off under reduced pressure. The obtained residue was purified by silica gel column chromatography and recrystallization to obtain Intermediate 5B (5.4 g, 86% yield). The molecular weight of the target compound was 500.54, and the mass spectrum of the resulting compound was analyzed as m/z (ratio of mass to charge)=500, thereby identified as the target compound.

### (9-3) Synthesis of Intermediate 5C

In the synthesize of Intermediate 1, Intermediate 5B (5.4 g) was used instead of Intermediate 1B to obtain Intermediate 5 (4.4 g, 92% yield). The molecular weight of the target compound was 444.43, and the mass spectrum of the resulting compound was analyzed as m/z (ratio of mass to charge)=444, thereby identified as the target compound.

### (9-4) Synthesis of Intermediate 5D

In the synthesize of Intermediate 1D, Intermediate 5C (5.1 g) was used instead of Intermediate 1C to obtain Intermediate 5D (3.9 g, 84% yield). The molecular weight of the target compound was 404.42, and the mass spectrum of the resulting compound was analyzed as m/z (ratio of mass to charge)=404, thereby identified as the target compound.

### (9-5) Synthesis of Intermediate 5E

In the synthesize of Intermediate 1E, Intermediate 5D (3.9 g) was used instead of Intermediate 1D to obtain Intermediate 5E (1.1 g, 16% yield). The molecular weight of the target compound was 668.53, and the mass spectrum of the resulting compound was analyzed as m/z (ratio of mass to charge)=668, thereby identified as the target compound.

### (9-6) Synthesis of Compound 5A

Under an argon atmosphere, a mixture of Intermediate 5E (0.75 g), di-p-tolylamine (0.48 g), dipalladium-tris(dibenzylideneacetone) chloroform complex (Pd₂(dba)₃-CHCl₃ (0.11 g), XPhos (0.21 g), cesium carbonate (1.46 g), xylenes (93 mL), and tert-butanol (18 mL) were stirred at 110° C. for 8 hours. The obtained reaction solution was cooled to room temperature, and extracted with toluene. The organic phase was washed with water, and dried over anhydrous sodium sulfate. The solvent was distilled off under reduced pressure. The obtained residue was purified by silica gel column chromatography to obtain Compound 5A (0.3 g, 33% yield). The molecular weight of Compound 5A was 762.95, and the mass spectrum of the resulting compound was analyzed as m/z (ratio of mass to charge)=762, thereby identified as the target compound.

### Example 10

### Synthesis of Compound 2B

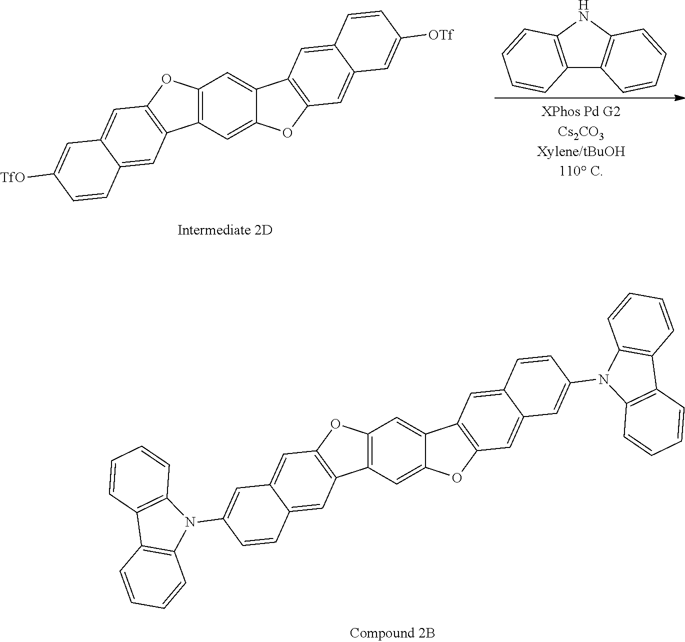 [SMILES: O=S(=O)(Oc1ccc2cc3c(cc2c1)oc1cc2c(cc13)oc1cc3cc(OS(=O)(=O)C(F)(F)F)ccc3cc12)C(F)(F)F ; c1ccc2c(c1)[nH]c1ccccc12 ; c1ccc2c(c1)c1ccccc1n2-c1ccc2cc3c(cc2c1)oc1cc2c(cc13)oc1cc3cc(-n4c5ccccc5c5ccccc54)ccc3cc12]

Under an argon atmosphere, a mixture of Intermediate 2D (200 mg), carbazole (128 mg), XPhos Pd G2 (manufactured by Sigma-Aldrich Co. LLC.) (38.5 mg), cesium carbonate (398 mg), xylenes (28 mL), and tert-butanol (12 mL) were stirred at 110° C. for 9 hours. The obtained reaction solution was cooled to room temperature, and extracted with toluene. The organic phase was washed with water, and dried over anhydrous sodium sulfate. The solvent was distilled off under reduced pressure. The resulting residue was purified by silica gel column chromatography to obtain Compound 2B (70 mg, 33% yield). The molecular weight of Compound 7 was 688.786, and the mass spectrum of the resulting compound was analyzed as m/z (ratio of mass to charge)=688, thereby identified as the target compound.

### Example 11

### Synthesis of Compound 2C

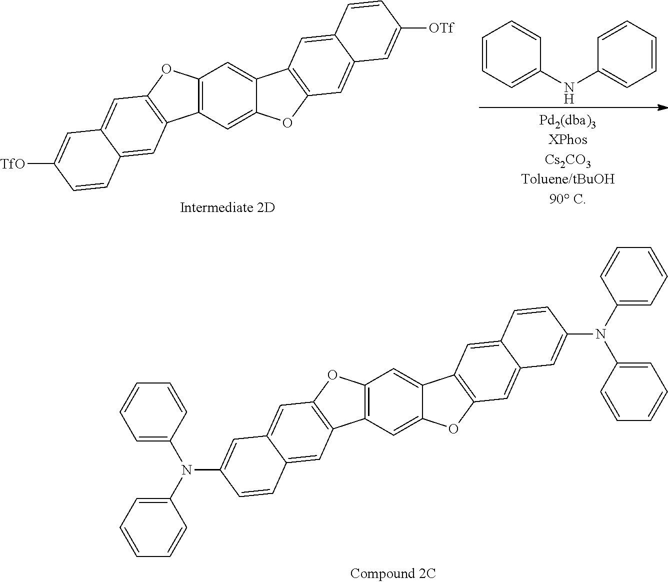 [SMILES: O=S(=O)(Oc1ccc2cc3c(cc2c1)oc1cc2c(cc13)oc1cc3cc(OS(=O)(=O)C(F)(F)F)ccc3cc12)C(F)(F)F ; c1ccc(Nc2ccccc2)cc1 ; c1ccc(N(c2ccccc2)c2ccc3cc4c(cc3c2)oc2cc3c(cc24)oc2cc4cc(N(c5ccccc5)c5ccccc5)ccc4cc23)cc1]

Under an argon atmosphere, a mixture of Intermediate 2D (4.0 g), diphenylamine (2.1 g), Pd₂(dba)₃ (0.56 g), XPhos (1.1 g), cesium carbonate (7.9 g), toluene (300 mL), tert-butanol (60 mL) was stirred at 90° C. for 4 hours. The obtained reaction solution was cooled to room temperature, and extracted with toluene. The organic phase was washed with water, and dried over anhydrous magnesium sulfate. The solvent was distilled off under reduced pressure. The resulting residue was purified by silica gel column chromatography and recrystallization to obtain Compound 2C (0.27 g, 25% yield). The molecular weight of the target compound was 692.818, and the mass spectrum of the obtained compound was analyzed as m/z (ratio of mass to charge)=692, thereby identified as the target compound.

### Example 12

### Synthesis of Compound 2D

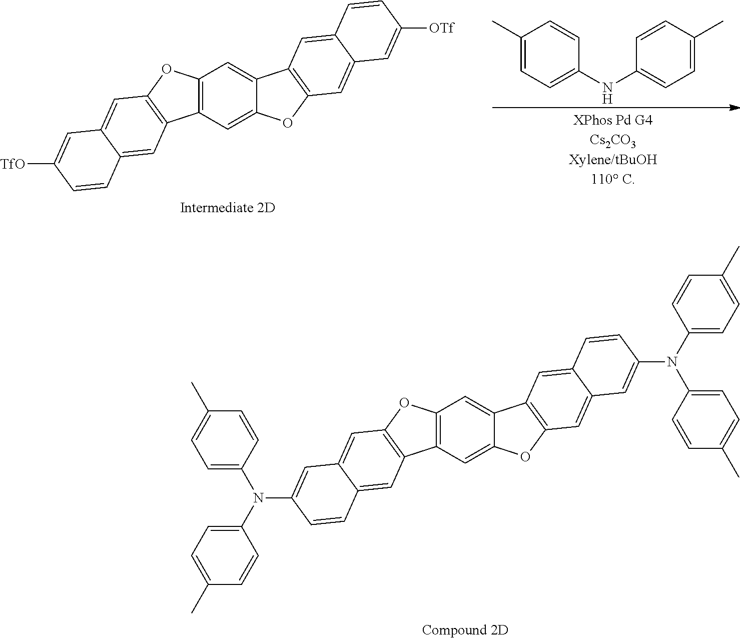

Under an argon atmosphere, a mixture of Intermediate 2D (0.68 g), di-p-tolylamine (0.62 g), XPhos Pd G4 (manufactured by Sigma-Aldrich Co. LLC.) (0.09 g), cesium carbonate (1.36 g), xylene (87 mL), and tert-butanol (18 mL) were stirred at 110° C. for 6 hours. The obtained reaction solution was cooled to room temperature, and extracted with toluene. The organic phase was washed with water, and dried over anhydrous sodium sulfate. The solvent was distilled off under reduced pressure. The resulting residue was purified by silica gel column chromatography and recrystallization to obtain Compound 2D (0.24 g, 30% yield). The molecular weight of the target compound was 748.926, and the mass spectrum of the resulting compound was analyzed as m/z (ratio of mass to charge)=748, thereby identified as the target compound.

### Example 13

### Synthesis of Compound 2E

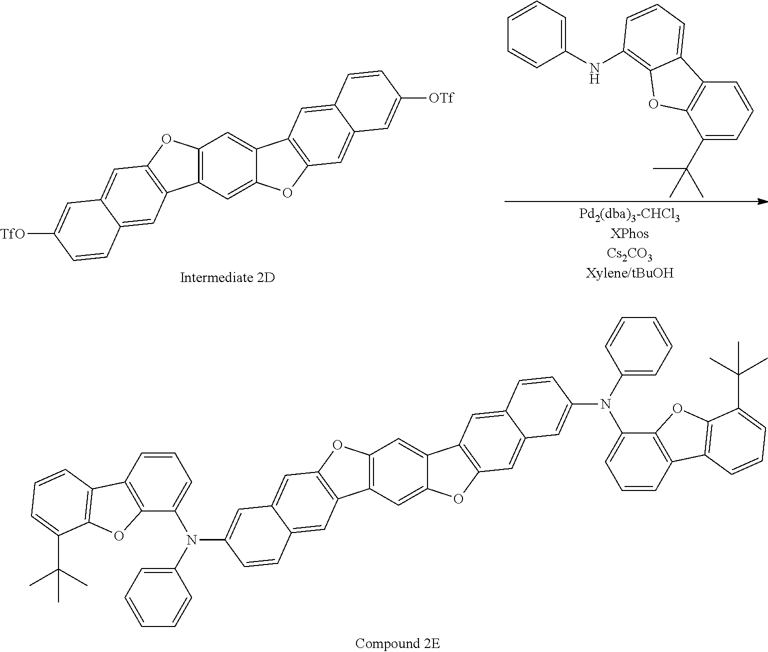 [SMILES: O=S(=O)(Oc1ccc2cc3c(cc2c1)oc1cc2c(cc13)oc1cc3cc(OS(=O)(=O)C(F)(F)F)ccc3cc12)C(F)(F)F ; CC(C)(C)c1cccc2c1oc1c(Nc3ccccc3)cccc12 ; CC(C)(C)c1cccc2c1oc1c(N(c3ccccc3)c3ccc4cc5c(cc4c3)oc3cc4c(cc35)oc3cc5cc(N(c6ccccc6)c6cccc7c6oc6c(C(C)(C)C)cccc67)ccc5cc34)cccc12]

Under an argon atmosphere, a mixture of Intermediate 2D (4.0 g), 6-tert-butyl-N-phenyldibenzo[b,d]furan-4-amine (4.9 g), Pd₂(dba)₃-CHCl₃ (0.64 g), XPhos (1.1 g), cesium carbonate (8.1 g), xylene (518 mL), and tert-butanol (104 mL) was stirred at 110° C. for 8 hours. The obtained reaction solution was cooled to room temperature, and filtered through a short column filled with silica gel. The solvent was distilled off under reduced pressure. The obtained residue was recrystallized by dioxane to obtain Compound 2E (1.6 g, 26% yield). The molecular weight of the target compound was 985.196, and the mass spectrum of the resulting compound was analyzed as m/z (ratio of mass to charge)=985, thereby identified as the target compound.

### Example 14

### Synthesis of Compound 2F

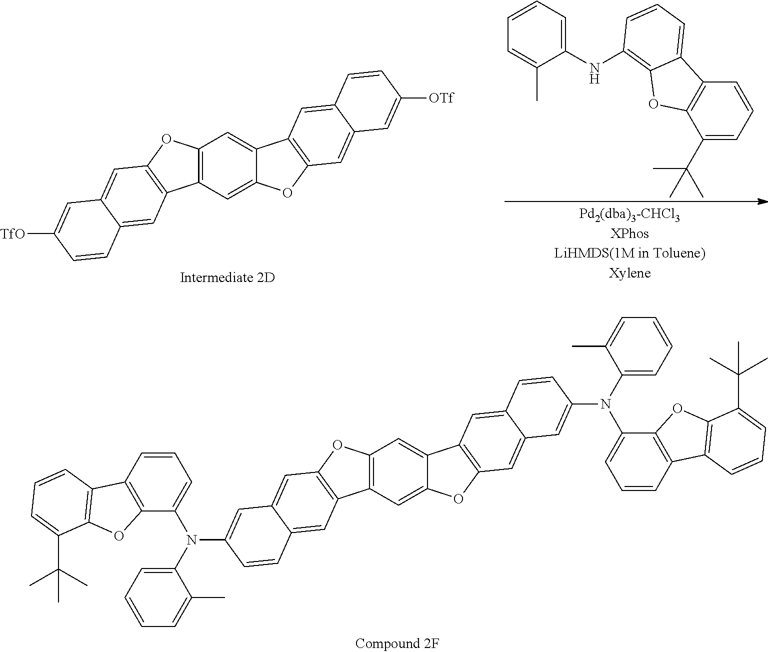 [SMILES: O=S(=O)(Oc1ccc2cc3c(cc2c1)oc1cc2c(cc13)oc1cc3cc(OS(=O)(=O)C(F)(F)F)ccc3cc12)C(F)(F)F ; Cc1ccccc1Nc1cccc2c1oc1c(C(C)(C)C)cccc12 ; Cc1ccccc1N(c1ccc2cc3c(cc2c1)oc1cc2c(cc13)oc1cc3cc(N(c4ccccc4C)c4cccc5c4oc4c(C(C)(C)C)cccc45)ccc3cc12)c1cccc2c1oc1c(C(C)(C)C)cccc12]

Under an argon atmosphere, a mixture of Intermediate 2D (3.3 g), 6-tert-butyl-N-(2-methylphenyl)dibenzo[b,d]furan-4-amine (3.7 g), Pd₂(dba)-CHCl₃ (0.53 g), XPhos (0.97 g),1 mol-toluene solution of lithium bis(tri-methylsilyl)amide (LiHMDS (1 M in Toluene)) (15 mL), and xylene (512 mL) was stirred at 90° C. for 8 hours. The obtained reaction solution was cooled to room temperature, and filtered through a short column filled with silica gel. The solvent was distilled off under reduced pressure. The obtained residue was recrystallized by dioxane to obtain Compound 2F (2.1 g, 40% yield). The molecular weight of the target compound was 1013.250, and the mass spectrum of the resulting compound was m/z (ratio of mass to charge)=1013, thereby identified as the target compound.

### Example 15

### Synthesis of Compound 2G

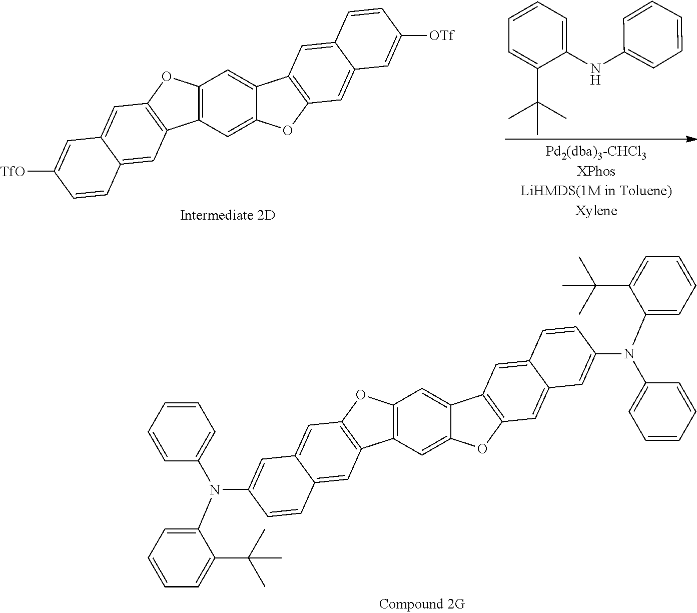 [SMILES: O=S(=O)(Oc1ccc2cc3c(cc2c1)oc1cc2c(cc13)oc1cc3cc(OS(=O)(=O)C(F)(F)F)ccc3cc12)C(F)(F)F ; CC(C)(C)c1ccccc1Nc1ccccc1 ; CC(C)(C)c1ccccc1N(c1ccccc1)c1ccc2cc3c(cc2c1)oc1cc2c(cc13)oc1cc3cc(N(c4ccccc4)c4ccccc4C(C)(C)C)ccc3cc12]

Under an argon atmosphere, a mixture of Intermediate 2D (6.4 g), 6-tert-butyl-N-(2-methylphenyl)dibenzo[b,d]furan-4-amine (4.8 g), Pd₂(dba)₃-CHCl₃ (0.50 g), XPhos (0.93 g), LiHMDS (1M in Toluene) (24 mL), xylene (500 mL) was stirred at 90° C. for 8 hours. The obtained reaction solution was cooled to room temperature, and filtered through a short column filled with silica gel. The solvent was distilled off under reduced pressure. The obtained residue was recrystallized by dioxane, dimethylformamide, dimethylacetamide, and the like to obtain Compound 2F (4.4 g, 55% yield). The molecular weight of the target compound was 805.034, and the mass spectrum of the resulting compound was analyzed as m/z (ratio of mass to charge)=805, thereby identified as the target compound.

### Example 16

### Synthesis of Compound 2H

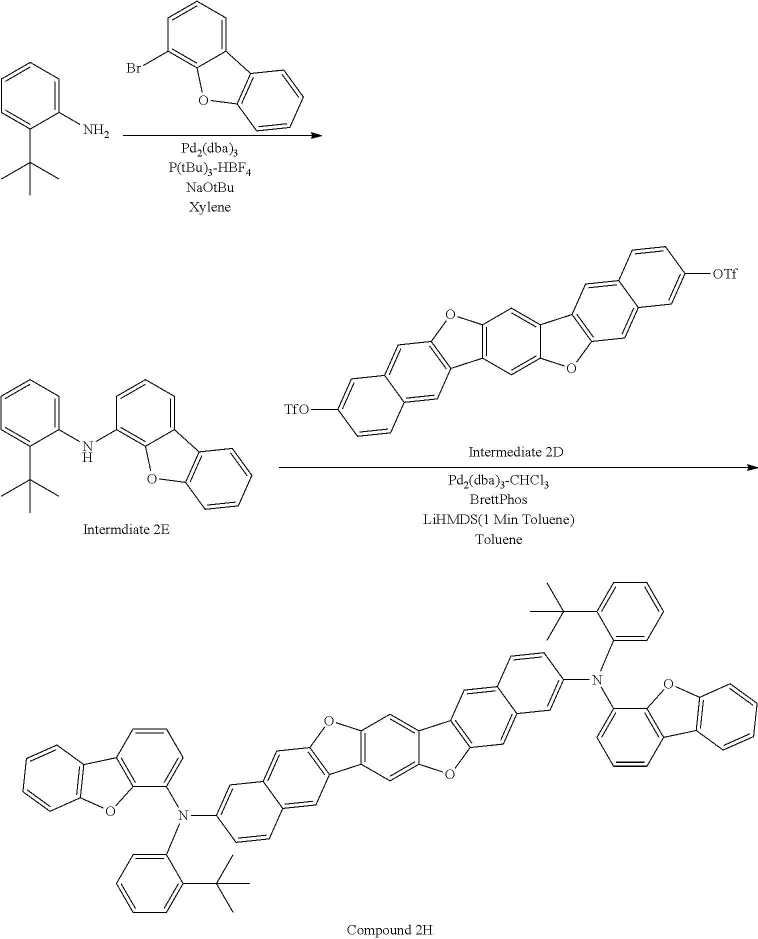 [SMILES: CC(C)(C)c1ccccc1N ; Brc1cccc2c1oc1ccccc12 ; CC(C)(C)c1ccccc1Nc1cccc2c1oc1ccccc12 ; O=S(=O)(Oc1ccc2cc3c(cc2c1)oc1cc2c(cc13)oc1cc3cc(OS(=O)(=O)C(F)(F)F)ccc3cc12)C(F)(F)F ; CC(C)(C)c1ccccc1N(c1ccc2cc3c(cc2c1)oc1cc2c(cc13)oc1cc3cc(N(c4ccccc4C(C)(C)C)c4cccc5c4oc4ccccc45)ccc3cc12)c1cccc2c1oc1ccccc12]

### (16-1) Synthesis of Intermediate 2E

Under an argon atmosphere, a mixture of 2-(tert-butylaniline (6.6 g), 4-bromodibenzo[b,d]furan (10 g), Pd₂(dba)₃ (0.37 g), tri-tert-butylphosphonium tetrafluoroborate (P(tBu)₃-HBF₄) (0.47 g), sodium tert-butoxide (5.8 g), xylene (100 mL) was stirred at 90° C. for 4 hours. The obtained reaction solution was cooled to room temperature, and extracted with toluene. The organic phase was washed with water, and dried over anhydrous sodium sulfate. The solvent was distilled off under reduced pressure. The obtained residue was purified by silica gel column chromatography to obtain Intermediate 2E (9.7 g, 77% yield). The molecular weight of the target compound was 315.416, and the mass spectrum of the resulting compound was analyzed as m/z (ratio of mass to charge)=315, thereby identified as the target compound.

### (16-2) Synthesis of Compound 2H

Under an argon atmosphere, a mixture of Intermediate 2D (4.2 g), Intermediate 2E (4.4 g), Pd₂(dba)₃-CHCl₃ (0.46 g), 2-(dicyclohexylphosphino)-3,6-dimetoxy-2′, 4′, 6′-triisopropyl-1,1′-biphenyl (BrettPhos) (0.96 g), LiHMDS (1M in Toluene) (16 mL), and toluene (320 mL) was stirred at 90° C. for 7 hours. The obtained reaction solution was cooled to room temperature, and filtered through a short column filled with silica gel. The solvent was distilled off under reduced pressure. The obtained residue was recrystallized by dioxane and cyclohexane to obtain Compound 2H (5.0 g, 79% yield). The molecular weight of the target compound was 985.196, and the mass spectrum of the resulting compound was analyzed as m/z (ratio of mass to charge)=985, thereby identified as the target compound.

### Example 17

### Synthesis of Compound 21

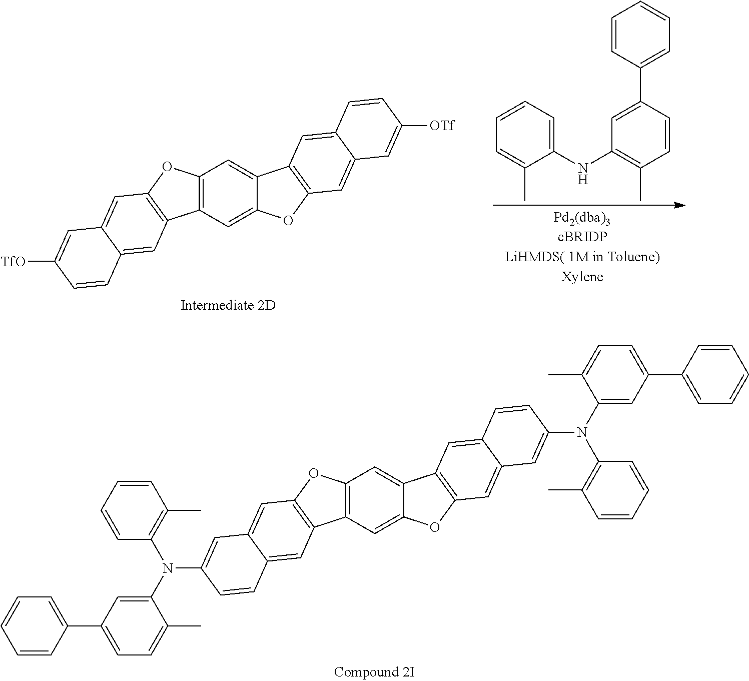 [SMILES: O=S(=O)(Oc1ccc2cc3c(cc2c1)oc1cc2c(cc13)oc1cc3cc(OS(=O)(=O)C(F)(F)F)ccc3cc12)C(F)(F)F ; Cc1ccccc1Nc1cc(-c2ccccc2)ccc1C ; Cc1ccccc1N(c1ccc2cc3c(cc2c1)oc1cc2c(cc13)oc1cc3cc(N(c4ccccc4C)c4cc(-c5ccccc5)ccc4C)ccc3cc12)c1cc(-c2ccccc2)ccc1C]

Under an argon atmosphere, a mixture of Intermediate 2D (3.0 g), 4-methyl-N-(2-methylphenyl[biphenyl]-3-amine (2.6 g), Pd₂(dba)₃ (0.084 g), di-tert-butyl(2,2-diphenyl-methyl-1-cyclopropyl)phosphine (cBRIDP) (0.12 g), LiHMDS (1M in Toluene) (18 mL), xylene (150 mL) was stirred at 110° C. for 3 hours. The obtained reaction solution was cooled to room temperature, and filtered through a short column filled with silica gel. The solvent was distilled off under reduced pressure. The obtained residue was recrystallized by isopropyl alcohol, dimethylacetamide, cyclohexanone or the like to obtain Compound 21 (0.85 g, 20% yield). The molecular weight of the target compound was 901.122, and the mass spectrum of the resulting compound was analyzed as m/z (ratio of mass to charge)=901, thereby identified as the target compound.

### Example 18

### Synthesis of Compound 2J

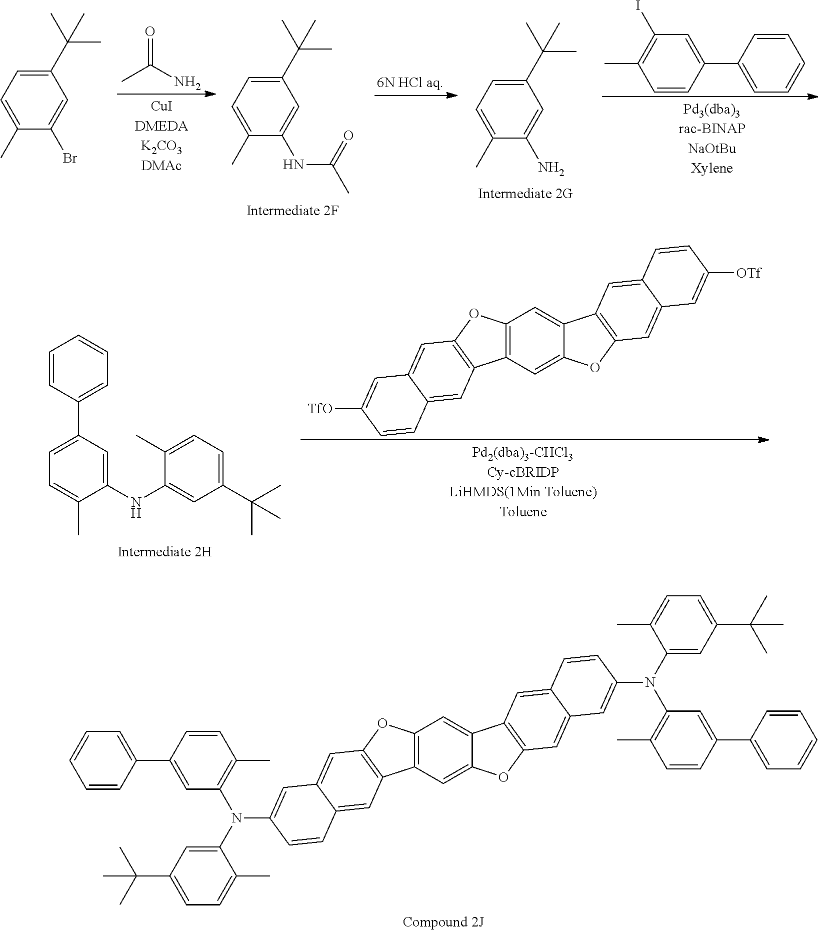

### (18-1) Synthesis of Intermediate 2F

Under an argon atmosphere, a mixture of 2-bromo-4-(tert-butyl)-1-methylbenzene (527 g), copper iodide(I) (Cul) (835 g), N,N′-dimethylethylenediamine (DMEDA) (193 g), acetamide (205 g), potassium carbonate (606 g), dimethylacetamide (DMAc) (7.9 L) was stirred at 120° C. for 14 hours. The obtained reaction solution was cooled to room temperature, and extracted with toluene. The organic phase was washed with water, and dried over anhydrous magnesium sulfate. The solvent was distilled off under reduced pressure. The obtained residue was suspended and washed with heptane with heating to obtain Intermediate 2F (130 g, 29% yield).

### (18-2) Synthesis of Intermediate 2G

Intermediate 2F (111 g) was stirred in hydrochloric acid (6N HCl aq.) at 100° C. for 15 hours. Toluene (450 mL) was slowly added to the obtained reaction solution, the solution was cooled to room temperature, and the precipitated solid was obtained by filtration. The solid obtained by filtration was added to an aqueous solution of sodium hydroxide, extracted with toluene, and dried over anhydrous magnesium sulfate. The solvent was distilled off under reduced pressure to obtain Intermediate 2G (85 g, 96% yield).

### Synthesis of (18-3) Intermediate 2H

Under an argon atmosphere, a mixture of Intermediate 2H (85 g), 3-iodo-4-methyl[biphenyl] (139 g), Pd₂(dba)₃ (6.5 g), (±)-(1,1′-binaphthalene-2,2′-diyl)bis(diphenylphosphine) (rac-BINAP) (8.8 g), sodium tert-butoxide (90 g), xylene (2.2 L) were stirred at 130° C. for 26 hours. The obtained reaction solution was cooled to room temperature. The organic phase was washed with water, and dried over anhydrous magnesium sulfate. The solvent was distilled off under reduced pressure. The resulting residue was purified by silica gel column chromatography and recrystallization to obtain Intermediate 2H (82 g, 52% yield). The molecular weight of the target compound was 329.487, and the mass spectrum of the resulting compound was analyzed as m/z (ratio of mass to charge)=329, thereby identified as the target compound.

### (18-4) Synthesis of Compound 2J

Under an argon atmosphere, a mixture of Intermediate 2D (3.1 g), Intermediate 2H (3.4 g), Pd₂(dba)₃-CHCl₃ (0.24 g), 1-(dicyclohexylphosphino)-2,2-diphenyl-1-methylcyclopropane (Cy-cBRIDP) (0.38 g), LiHMDS (1M in Toluene) (11 mL), toluene (237 mL) was stirred at 100° C. for 8 hours. The obtained reaction solution was cooled to room temperature, and filtered through a short column filled with silica gel. The solvent was distilled off under reduced pressure. The obtained residue was recrystallized by cyclohexane, dimethylacetamide, tert-amyl alcohol or the like to obtain Compound 2J (0.67 g, 13% yield). The molecular weight of the target compound was 1013.338, and the mass spectrum of the resulting compound was analyzed as m/z (ratio of mass to charge)=1013, thereby identified as the target compound.

### Example 19

### Synthesis of Compound 2K

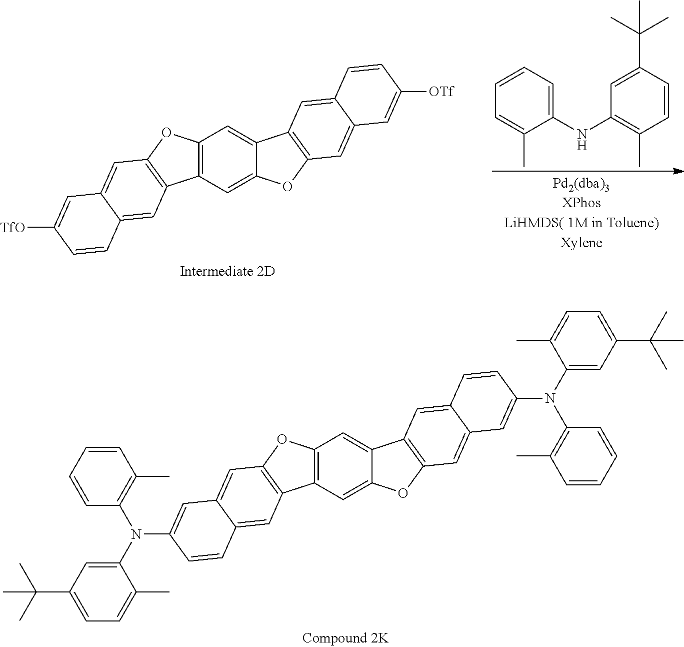 [SMILES: O=S(=O)(Oc1ccc2cc3c(cc2c1)oc1cc2c(cc13)oc1cc3cc(OS(=O)(=O)C(F)(F)F)ccc3cc12)C(F)(F)F ; Cc1ccccc1Nc1cc(C(C)(C)C)ccc1C ; Cc1ccccc1N(c1ccc2cc3c(cc2c1)oc1cc2c(cc13)oc1cc3cc(N(c4ccccc4C)c4cc(C(C)(C)C)ccc4C)ccc3cc12)c1cc(C(C)(C)C)ccc1C]

Under an argon atmosphere, a mixture of Intermediate 2D (2.0 g), 5-tert-butyl-2-methyl-N-(2-methylphenyl)aniline (1.6 g), Pd₂(dba)₃ (0.056 g), XPhos (0.11 g), LiHMDS (1M in Toluene) (12 mL), xylene (200 mL) was stirred at 110° C. for 3 hours. The obtained reaction solution was cooled to room temperature, and the solvent was distilled off under reduced pressure. The obtained residue was purified by silica gel column chromatography and recrystallization to obtain Compound 2K (0.68 g, 25% yield). The molecular weight of the target compound was 861.142, and the mass spectrum of the resulting compound was analyzed as m/z (ratio of mass to charge)=861, thereby identified as the target compound.

### Example 20

### Synthesis of Compound 3B

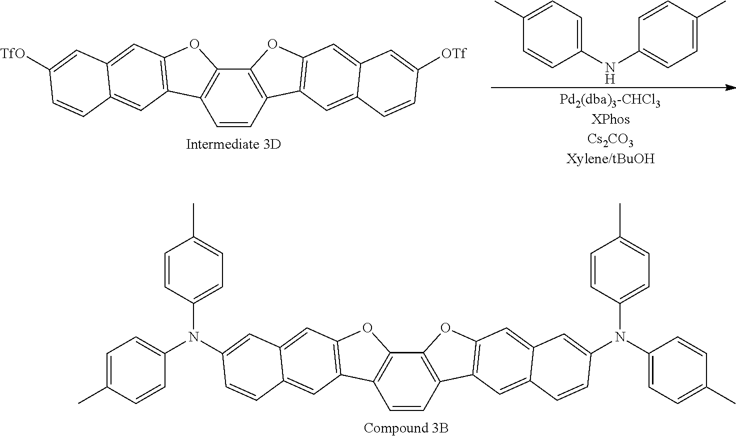

Under an argon atmosphere, a mixture of Intermediate 3D (6.5 g), di-p-tolylamine (4.9 g), Pd₂(dba)₃-CHCl₃ (1.5 g), XPhos (2.8 g), cesium carbonate (13 g), xylene (333 mL), and tert-butanol (66 mL) were stirred at 110° C. for 6 hours. The obtained reaction solution was cooled to room temperature. The organic phase was washed with water, and dried over anhydrous sodium sulfate. The solvent was distilled off under reduced pressure. The obtained residue was purified by silica gel column chromatography and recrystallization to obtain Compound 3B (2.2 g, 29% yield). The molecular weight of the target compound was 748.926, and the mass spectrum of the resulting compound was analyzed as m/z (ratio of mass to charge)=748, thereby identified as the target compound.

### Example 21

### Synthesis of Compound 3C

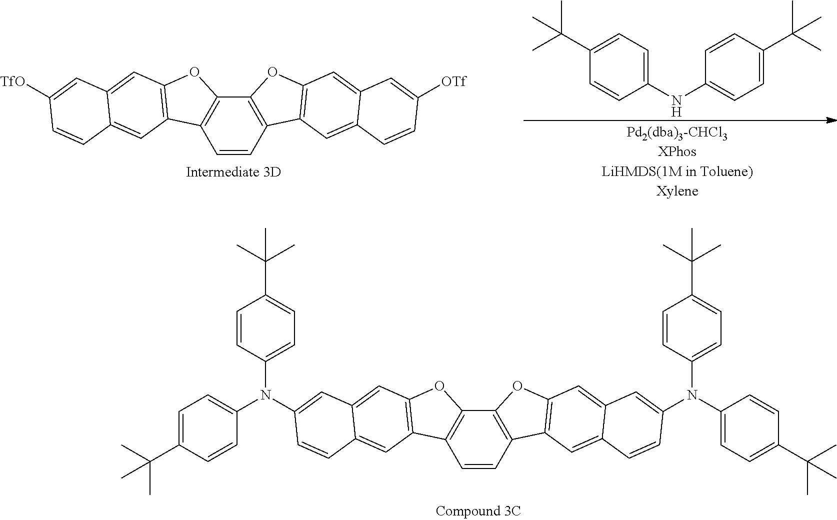 [SMILES: O=S(=O)(Oc1ccc2cc3c(cc2c1)oc1c3ccc2c3cc4ccc(OS(=O)(=O)C(F)(F)F)cc4cc3oc21)C(F)(F)F ; CC(C)(C)c1ccc(Nc2ccc(C(C)(C)C)cc2)cc1 ; CC(C)(C)c1ccc(N(c2ccc(C(C)(C)C)cc2)c2ccc3cc4c(cc3c2)oc2c4ccc3c4cc5ccc(N(c6ccc(C(C)(C)C)cc6)c6ccc(C(C)(C)C)cc6)cc5cc4oc32)cc1]

Under an argon atmosphere, a mixture of Intermediate 3D (3.3 g), bis-(4-(tert-butyl)phenyl)amine (3.1 g), Pd₂(dba)₃-CHCl₃ (0.26 g), XPhos (0.48 g), LiHMDS (1M in Toluene) (12 mL), and xylene (504 mL) was stirred at 110° C. for 6 hours. The obtained reaction solution was cooled to room temperature, and filtered through a short column filled with silica gel. The solvent was distilled off under reduced pressure. The obtained residue was recrystallized by cyclohexane or dioxane to obtain Compound 3C (3.6 g, 77% yield). The molecular weight of the target compound was 917.250, and the mass spectrum of the resulting compound was analyzed as m/z (ratio of mass to charge)=917, thereby identified as the target compound.

### Example 22

### Synthesis of Compound 3D

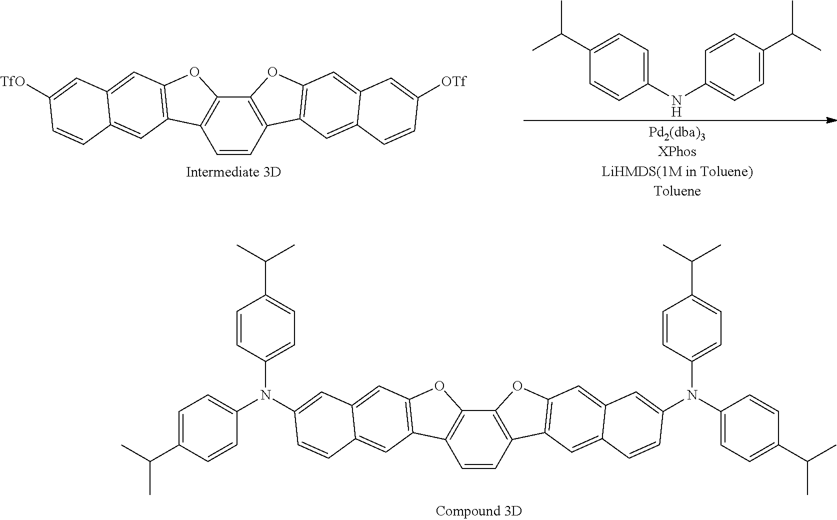 [SMILES: O=S(=O)(Oc1ccc2cc3c(cc2c1)oc1c3ccc2c3cc4ccc(OS(=O)(=O)C(F)(F)F)cc4cc3oc21)C(F)(F)F ; CC(C)c1ccc(Nc2ccc(C(C)C)cc2)cc1 ; CC(C)c1ccc(N(c2ccc(C(C)C)cc2)c2ccc3cc4c(cc3c2)oc2c4ccc3c4cc5ccc(N(c6ccc(C(C)C)cc6)c6ccc(C(C)C)cc6)cc5cc4oc32)cc1]

Under an argon atmosphere, a mixture of Intermediate 3D (3.2 g), bis(4-isopropylphenyl)amine (2.7 g), Pd₂(dba)₃ (0.22 g), XPhos (0.47 g), LiHMDS (1M in Toluene) (12.5 mL), and toluene (500 mL) was stirred at 110° C. for 4 hours. The obtained reaction solution was cooled to room temperature, and filtered through a short column filled with silica gel. The solvent was distilled off under reduced pressure. The obtained residue was recrystallized from dioxane to obtain Compound 3D (1.6 g, 39% yield). The molecular weight of the target compound was 861.142, and the mass spectrum of the resulting compound was analyzed as m/z (ratio of mass to charge)=861, thereby identified as the target compound.

### Example 23

### Synthesis of Compound 3E

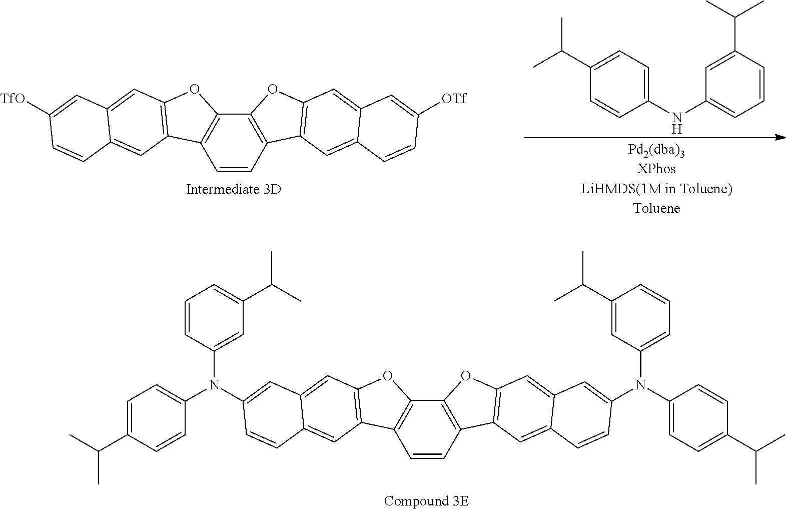 [SMILES: O=S(=O)(Oc1ccc2cc3c(cc2c1)oc1c3ccc2c3cc4ccc(OS(=O)(=O)C(F)(F)F)cc4cc3oc21)C(F)(F)F ; CC(C)c1ccc(Nc2cccc(C(C)C)c2)cc1 ; CC(C)c1ccc(N(c2cccc(C(C)C)c2)c2ccc3cc4c(cc3c2)oc2c4ccc3c4cc5ccc(N(c6ccc(C(C)C)cc6)c6cccc(C(C)C)c6)cc5cc4oc32)cc1]

Under an argon atmosphere, a mixture of Intermediate 3D (5.5 g), 3-(propane-2-yl-N-[4-(propane-2-ylphenyl]aniline (4.7 g), Pd₂(dba)₃-CHCl₃ (0.43 g), XPhos (0.80 g), LiHMDS (1M in Toluene)(21 mL), and toluene (500 mL) was stirred at 70° C. for 7 hours. The obtained reaction solution was cooled to room temperature, and filtered through a short column filled with silica gel. The solvent was distilled off under reduced pressure. The obtained residue was recrystallized by dioxane, tert-butanol, ethyl acetate, cyclohexane, isopropyl alcohol, chloroform or the like to obtain Compound 3E (2.9 g, 39% yield). The molecular weight of the target compound was 861.142, and the mass spectrum of the resulting compound was analyzed as m/z (ratio of mass to charge)=861, thereby identified as the target compound.

### Example 24

### Synthesis of Compound 3F

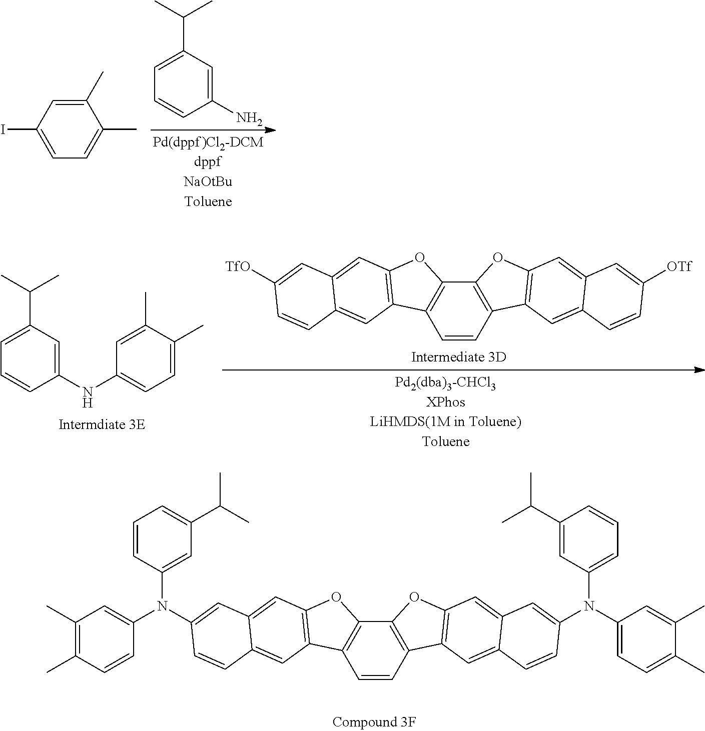

### (24-1) Synthesis of Intermediate 3E

Under an argon atmosphere, a mixture of 4-iodo-1,2-dimethylbenzene (10 g), 3-isopropylaniline (6.4 g), dichloro[1,1′-bis(diphenylphosphino)ferrocene]palladium(II) dichloromethane adduct (Pd(dppf)Cl₂-DCM) (0.35 g), 1,1′-bis(diphenylphosphino)ferrocene (dppf) (0.72 g), sodium tert-butoxide (6.2 g), and toluene (174 mL) was stirred at 130° C. for 7 hours. The obtained reaction solution was cooled to room temperature, filtered through celite, and the solvent was distilled off under reduced pressure. The resulting residue was purified by silica gel column chromatography to obtain Intermediate 3E (10 g, 96% yield). The molecular weight of the target compound was 239.362, and the mass spectrum of the resulting compound was analyzed as m/z (ratio of mass to charge)=239, thereby identified as the target compound.

### (24-2) Synthesis of Compound 3F

Under an argon atmosphere, a mixture of Intermediate 2D (5.8 g), Intermediate 3E (4.7 g), Pd₂(dba)-CHCl₃ (0.92 g), XPhos (1.7 g), LiHMDS (1 Min Toluene) (22 mL), and toluene (500 mL) were stirred at 70° C. for 7 hours. The obtained reaction solution was cooled to room temperature, and filtered through a short column filled with silica gel. The solvent was distilled off under reduced pressure. The obtained residue was recrystallized by dioxane, cyclohexane, dibutyl ether, chloroform, or the like to obtain Compound 3F (2.1 g, 28% yield). The molecular weight of the target compound was 833.088, and the mass spectrum of the resulting compound was analyzed as m/z (ratio of mass to charge)=833, thereby identified as the target compound.

### Comparative Examples 1 to 3

Using the following Comparative Example Compounds 1 to 3, toluene solutions thereof were prepared and evaluated in the same manner as in Example 1. The results are shown in Table 1.

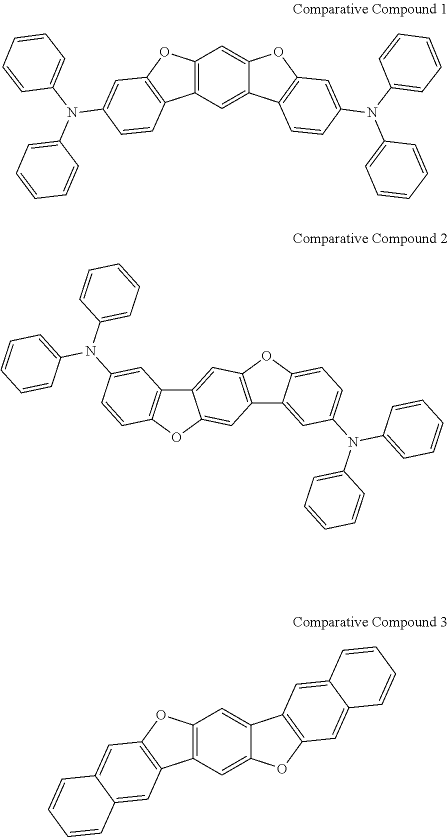 [SMILES: c1ccc(N(c2ccccc2)c2ccc3c(c2)oc2cc4oc5cc(N(c6ccccc6)c6ccccc6)ccc5c4cc23)cc1 ; c1ccc(N(c2ccccc2)c2ccc3oc4cc5c(cc4c3c2)oc2ccc(N(c3ccccc3)c3ccccc3)cc25)cc1 ; c1ccc2cc3c(cc2c1)oc1cc2c(cc13)oc1cc3ccccc3cc12]

TABLE 1

| | | PLQY | FL-peak |
|---|---|---|---|
| | Compound | (%) | (nm) |
| Example 1 | Compound 1A | 80 | 427 |
| Example 2 | Compound 1B | 95 | 437 |
| Example 3 | Compound 1C | 86 | 445 |
| Example 4 | Compound 2A | 91 | 449 |
| Example 5 | Compound 3A | 92 | 440 |
| Example 6 | Compound 4A | 89 | 443 |
| Example 7 | Compound 1D | 92 | 437 |
| Example 8 | Compound 1E | 83 | 439 |
| Example 9 | Compound 5A | 81 | 435 |
| Example 10 | Compound 2B | 84 | 437 |
| Example 11 | Compound 2C | 92 | 455 |
| Example 12 | Compound 2D | 89 | 465 |
| Example 13 | Compound 2E | 91 | 451 |
| Example 14 | Compound 2F | 79 | 447 |
| Example 15 | Compound 2G | 90 | 450 |
| Example 16 | Compound 2H | 82 | 444 |
| Example 17 | Compound 2I | 92 | 448 |
| Example 18 | Compound 2J | 87 | 450 |
| Example 19 | Compound 2K | 86 | 449 |
| Example 20 | Compound 3B | 89 | 451 |
| Example 21 | Compound 3C | 93 | 451 |
| Example 22 | Compound 3D | 89 | 451 |
| Example 23 | Compound 3E | 87 | 449 |
| Example 24 | Compound 3F | 92 | 450 |
| Comp. Ex. 1 | Comparative | 64 | 397 |
| | Compound 1 | | |
| Comp. Ex. 2 | Comparative | 37 | 428 |
| | Compound 2 | | |
| Comp. Ex. 3 | Comparative | 55 | 395 |
| | Compound 3 | | |

In Examples 1 to 24, values of fluorescent quantum yield (PLQY) were higher than those in Comparative Examples 1 to 3. In Examples 1 to 24, fluorescence peak wavelengths (FL-peak) were longer than those in Comparative Examples 1 to 3, and fluorescence spectra with high blue purities were obtained.

### Example 25

### <Fabrication of Organic EL Device>

A25 mm×75 mm×1.1 mm-thick glass substrate with an ITO transparent electrode (anode) (manufactured by GEOMATEC Co., Ltd.) was subjected to ultrasonic cleaning in isopropyl alcohol for 5 minutes, and then subjected to UV-ozone cleaning for 30 minutes. The thickness of the ITO film was 130 nm.

The glass substrate alter being cleaned was mounted onto a substrate holder in a vacuum vapor deposition apparatus, and Compound HI-1 was deposited on a surface on the side on which the transparent electrode line was formed so as to cover the transparent electrode to form a hole-injecting layer having a thickness of 5 nm.

Compound HT-1 was deposited on the hole-injecting layer film to form a first hole-transporting layer having a thickness of 80 nm.

Subsequently, Compound HT-2 was vapor-deposited on the first hole-transporting layer to form a second hole-transporting layer having a thickness of 10 nm.

Subsequently, a 25 nm-thick emitting layer was formed on the second hole-transporting layer by co-evaporation of Compound 2E (dopant material) and Compound BH-1 (host material) so that the ratio (weight ratio) of the dopant material was 2%.

Then, ET-1 was vapor-deposited on the emitting layer to form a first electron-transporting layer having a thickness of 10 nm.

Then, ET-2 was vapor-deposited on the first electron-transporting layer to form a second electron-transporting layer having a thickness of 15 nm.

Lithium fluoride (LiF) was vapor-deposited on the second electron-transporting layer to form electron injecting electrodes having a thickness of 1 nm.

Metallic aluminum (A) was deposited on electron-injecting electrode to form a metal cathode having a thickness of 80 nm.

### <Evaluation of Organic EL Device>

Initial characteristics of the obtained organic EL devices were measured by driving at a constant current of 10 mA/cm² of DC (direct current) at room temperature.

Voltage was applied to the organic EL device to be 10 mA/cm² in current density, thereby measuring an EL emission spectrum by using Spectroradiometer CS-1000 (manufactured by Konica Minolta, Inc.). External quantum efficiency (EQE) (%) was calculated from the obtained spectral radiance spectrum. The results are shown in Table 2.

Voltage was applied to the organic EL device to be 50 mA/cm² in current density, and the lifetime LT95 (h) until the luminance becomes 95% of the initial luminance. The results are shown in Table 2.

### Examples 26 to 32 and Comparative Examples 4 to 10

The organic EL devices were fabricated and evaluated in the same manner as in Example 25 except that the host material (BH) and the dopant material (BD) shown in the following Table 2 were used. The results are shown in Table 2.

The compounds used for fabricating the organic EL devices of Examples 25 to 32 and Comparative Examples 4 to 10 are shown below.

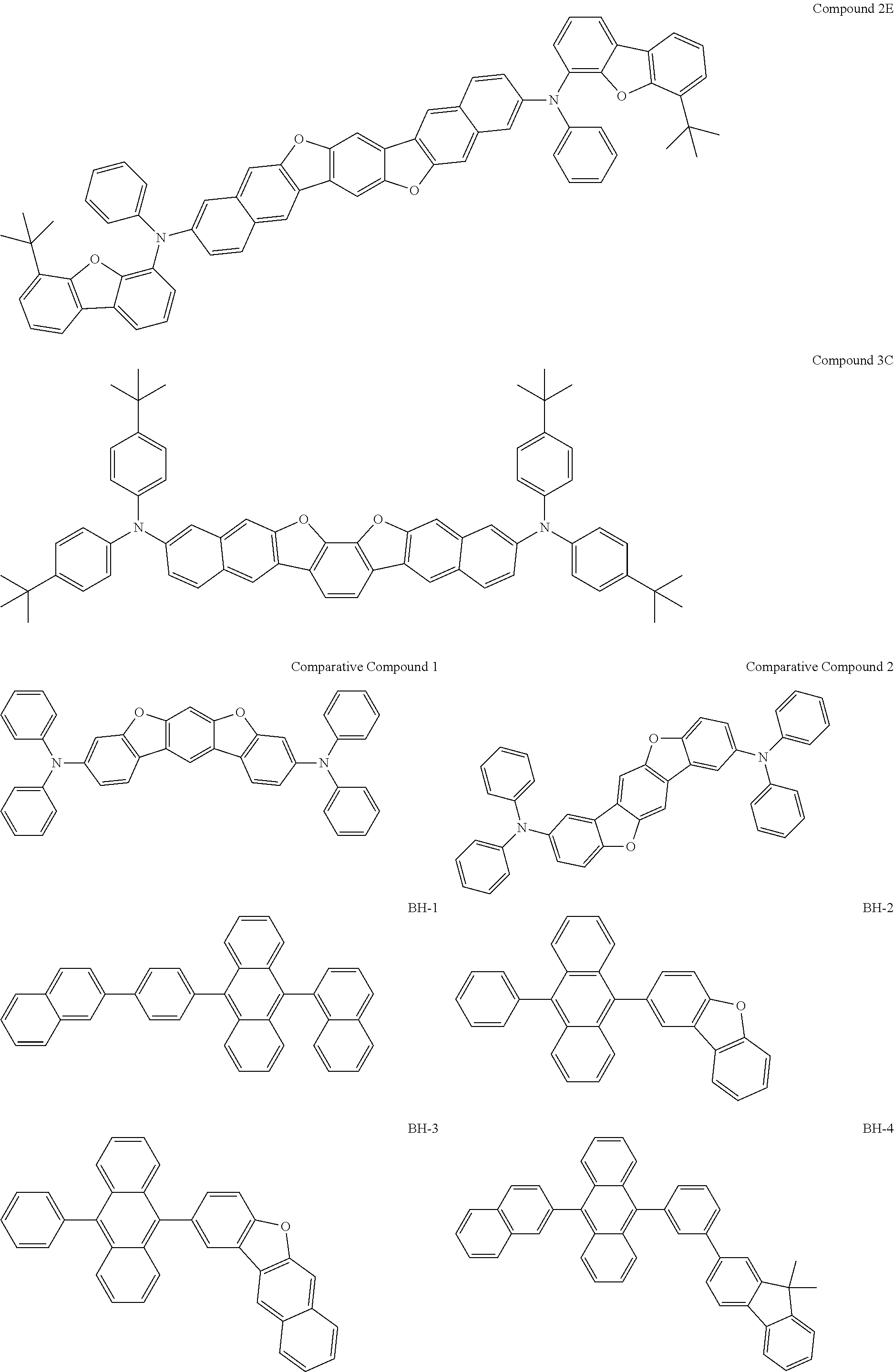

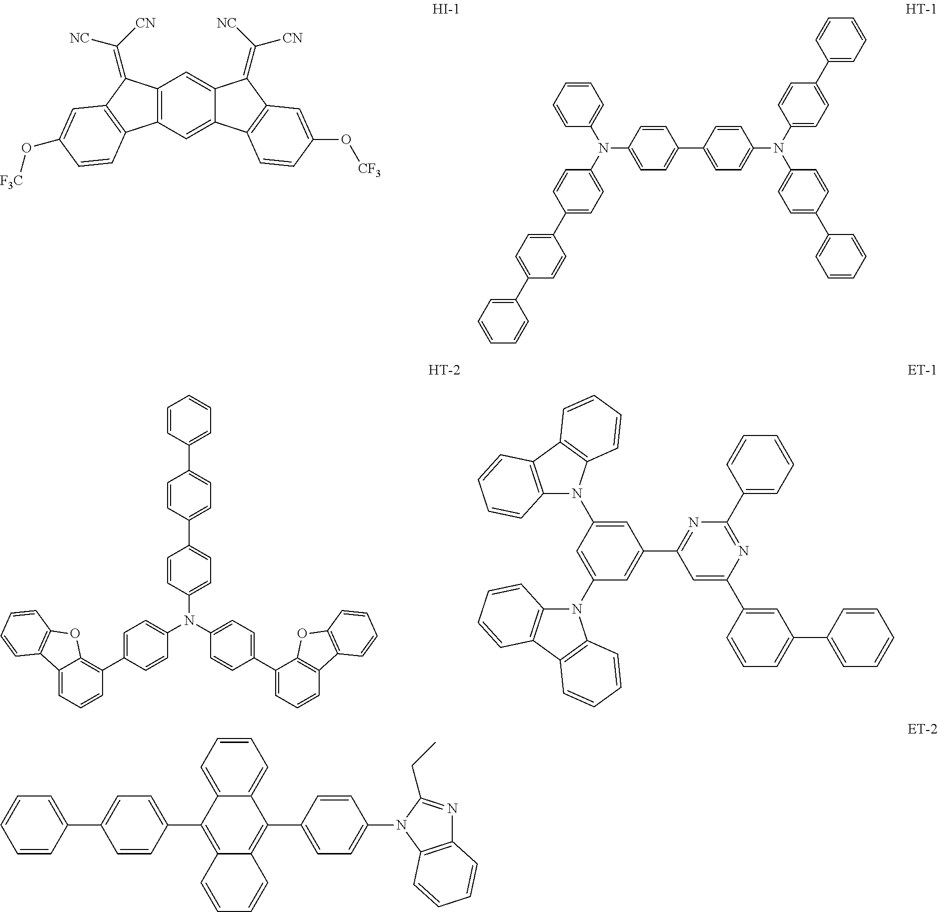 [SMILES: N#CC(C#N)=C1c2cc(OC(F)(F)F)ccc2-c2cc3c(cc21)C(=C(C#N)C#N)c1cc(OC(F)(F)F)ccc1-3 ; c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1 ; c1ccc(-c2ccc(-c3ccc(N(c4ccc(-c5cccc6c5oc5ccccc56)cc4)c4ccc(-c5cccc6c5oc5ccccc56)cc4)cc3)cc2)cc1 ; c1ccc(-c2cccc(-c3cc(-c4cc(-n5c6ccccc6c6ccccc65)cc(-n5c6ccccc6c6ccccc65)c4)nc(-c4ccccc4)n3)c2)cc1 ; CCc1nc2ccccc2n1-c1ccc(-c2c3ccccc3c(-c3ccc(-c4ccccc4)cc3)c3ccccc23)cc1]

TABLE 2

| | BH | BD | EQE (%) | LT95(h) |
|---|---|---|---|---|
| Example 25 | BH-1 | Compound 2E | 8.9 | 136 |
| Example 26 | | Compound 3C | 9.7 | 165 |
| Comp. Ex. 4 | | Comparative | 6.0 | 7 |
| | | Compound 1 | | |
| Comp. Ex. 5 | | Comparative | 5.9 | 8 |
| | | Compound 2 | | |
| Example 27 | BH-2 | Compound 2E | 7.3 | 61 |
| Example 28 | | Compound 3C | 7.2 | 145 |
| Comp. Ex. 6 | | Comparative | 3.2 | 15 |
| | | Compound 1 | | |
| Comp. Ex. 7 | | Comparative | 3.5 | 6 |
| | | Compound 2 | | |
| Example 29 | BH-3 | Compound 2E | 7.1 | 44 |
| Example 30 | | Compound 3C | 7.0 | 123 |
| Comp. Ex. 8 | | Comparative | 3.0 | 12 |
| | | Compound 1 | | |
| Comp. Ex. 9 | | Comparative | 3.1 | 4 |
| | | Compound 2 | | |
| Example 31 | BH-4 | Compound 2E | 6.3 | 55 |
| Example 32 | | Compound 3C | 6.2 | 133 |
| Comp. Ex. 10 | | Comparative | 2.0 | 13 |
| | | Compound 1 | | |
| Comp. Ex. 11 | | Comparative | 2.5 | 5 |
| | | Compound 2 | | |

From the results of Table 2, it is understood that the organic EL devices of Examples 25 to 32 using compound represented by the formula (1) as a dopant material for an emitting layer not only have a remarkably higher external quantum efficiency (EQE) than the devices of Comparative Examples 4 to 11, but also the device lifetime is greatly improved.

Although only some exemplary embodiments and/or examples of this invention have been described in detail above, those skilled in the art will readily appreciate that many modifications are possible in the exemplary embodiments and/or examples without materially departing from the novel teachings and advantages of this invention. Accordingly, all such modifications are intended to be included within the scope of this invention.

The documents described in the specification are incorporated herein by reference in its entirety.

1. A compound represented by the following formula (1): 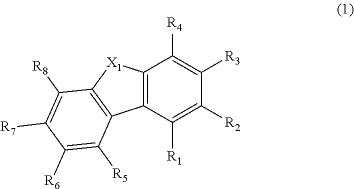
wherein in the formula (1), one pair among R₁ and R₂, R₂ and R₃, and R₃ and R₄ is respectively bonded with a divalent group represented by the formula (11): 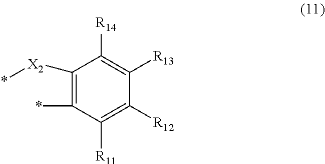
wherein X₁ and X₂ are independently O, S or C(R′)₂;
at least one pair among R₁₁ and R₁₂, R₁₂ and R₁₃, and R₁₃ and R₁₄ is respectively bonded with a divalent group represented by the following formula (12a);
at least one pair among R₅ and R₆, R₆ and R₇, and R₇ and R₈ is respectively bonded with a divalent group represented by the following formula (12b);
when a plurality of divalent groups represented by the following formulas (12a) and (12b) are present, the plurality of divalent groups represented by the following formulas (12a) and (12b) may be the same or different; 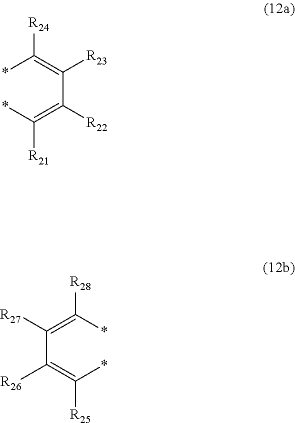
wherein R′, R₂₁ to R₂₈, R₁ to R₄ which are not bonded with a divalent group represented by the formula (11), R₁₁ to R₁₄ which are not bonded with a divalent group represented by the formula (12a), and R₅ to R₈ which are not bonded with a divalent group represented by the formula (12b) independently represent a hydrogen atom, a substituted or unsubstituted alkyl group including 1 to 50 carbon atoms, a substituted or unsubstituted haloalkyl group including 1 to 50 carbon atoms, a substituted or unsubstituted alkenyl group including 2 to 50 carbon atoms, a substituted or unsubstituted alkynyl group including 2 to 50 carbon atoms, a substituted or unsubstituted cycloalkyl group including 3 to 50 ring carbon atoms, a substituted or unsubstituted alkoxy group including 1 to 50 carbon atoms, a substituted or unsubstituted alkylthio group including 1 to 50 carbon atoms, a substituted or unsubstituted aryloxy group including 6 to 50 ring carbon atoms, a substituted or unsubstituted arylthio group including 6 to 50 ring carbon atoms, a substituted or unsubstituted aralkyl group including 7 to 50 carbon atoms, —Si(R₃₁)(R₃₂)(R₃₃), —C(═O)R₃₄, —COOR₃₅, a halogen atom, a cyano group, a nitro group, a substituted or unsubstituted aryl group including 6 to 50 ring carbon atoms, a substituted or unsubstituted monovalent heterocyclic group including 5 to 50 ring atoms, or a group represented by the following formula (13);
provided that at least one of R₂₁ to R₂₈, R₁ to R₄ which are not bonded with a divalent group represented by the formula (11), R₁₁ to R₁₄ which are not bonded with a divalent group represented by the formula (12a), and R₅ to R₈ which are not bonded with a divalent group represented by the formula (12b) is a group represented by the following formula (13);
two R's may be the same or different;
R₃₁ to R₃₅ are independently a hydrogen atom, a substituted or unsubstituted alkyl group including 1 to 50 carbon atoms, a substituted or unsubstituted aryl group including 6 to 50 ring carbon atoms, or a substituted or unsubstituted monovalent heterocyclic group including 5 to 50 ring atoms;
when a plurality of R₃₁ to R₃₅ are present, the plurality of R₃₁ to R₃₅ may be independently the same or different;
when a plurality of groups represented by the following formula (13) are present, the plurality of groups represented by the following formula (13) may be the same or different; 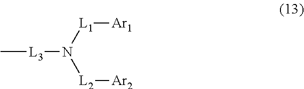
wherein in the formula (13),
L₁ to L₃ are independently a single bond, a substituted or unsubstituted alkylene group including 1 to 30 carbon atoms, a substituted or unsubstituted arylene group including 6 to 30 ring carbon atoms, or a substituted or unsubstituted divalent heterocyclic group including 5 to 30 ring atoms;
Ar₁ and Ar₂ are independently a substituted or unsubstituted alkyl group including 1 to 50 carbon atoms, a substituted or unsubstituted aryl group including 6 to 50 ring carbon atoms or a substituted or unsubstituted monovalent heterocyclic group including 5 to 50 ring atoms, and Ar₁ and Ar₂ may be bonded with each other via a single bond or via —O—, —S— or —C(R)₂—; and
R is a substituent, and two Rs may be the same or different.

2. The compound according to claim 1, wherein the compound represented by the formula (1) is selected from the group consisting of compounds represented by the following formulas (1-1H) to (1-6H): 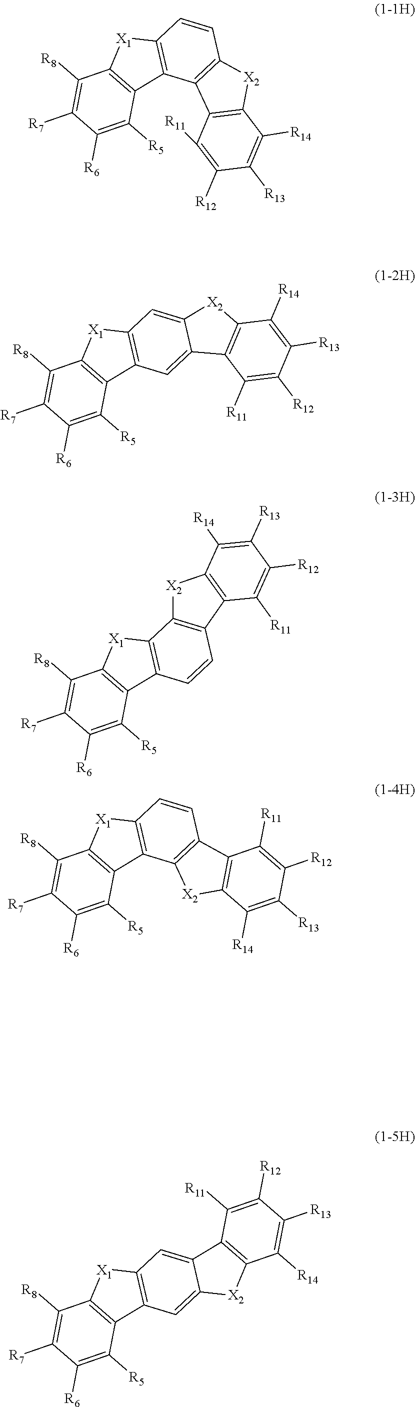 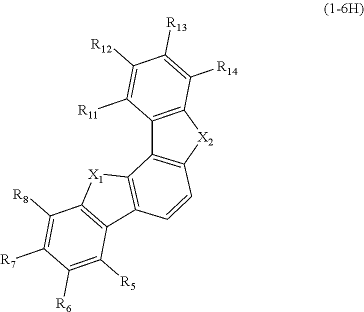
wherein in the formulas (1-1H) to (1-6H), X₁, X₂, R₅ to R₈ and R₁₁ to R₁₄ are as defined in the formula (1).

3. The compound according to claim 2, wherein the compound represented by the formula (1) is a compound represented by the formula (1-2H).

4. The compound according to claim 1, wherein the divalent group represented by the formula (11) is selected from the group consisting of divalent groups represented by the following formulas (11-1H) to (11-3H): 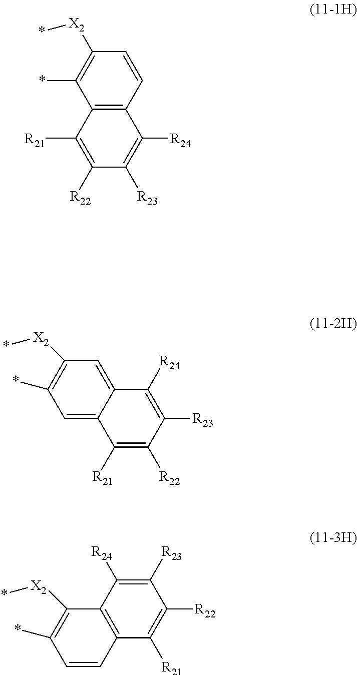
wherein in the formulas (11-1H) to (11-3H), X₂ and R₂₁ to R₂₄ are as defined in the formula (1).

5. The compound according to claim 4, wherein the group represented by the formula (11) is a divalent group represented by the formula (11-2H).

6. The compound according to claim 1, wherein the compound represented by the formula (1) is selected from the group consisting of compounds represented by the following formulas (1-21) to (1-23): 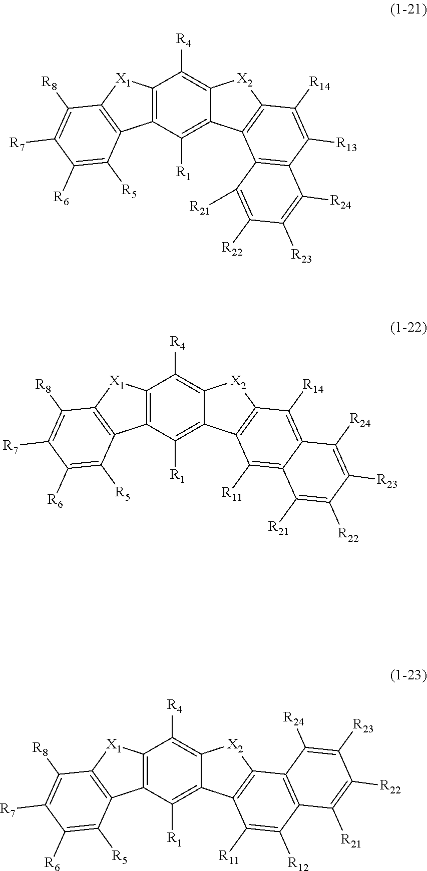
wherein in the formulas (1-21) to (1-23), X₁, X₂, R₁, R₄ to R₈, R₁₁ to R₁₄ and R₂₁ to R₂₄ are as defined in the formula (1).

7. The compound according to claim 6, wherein the compound represented by the formula (1) is a compound represented by the formula (1-22).

8. The compound according to claim 1, wherein the compound represented by the formula (1) is selected from the group consisting of compounds represented by the following formulas (1-11H) to (1-13H): 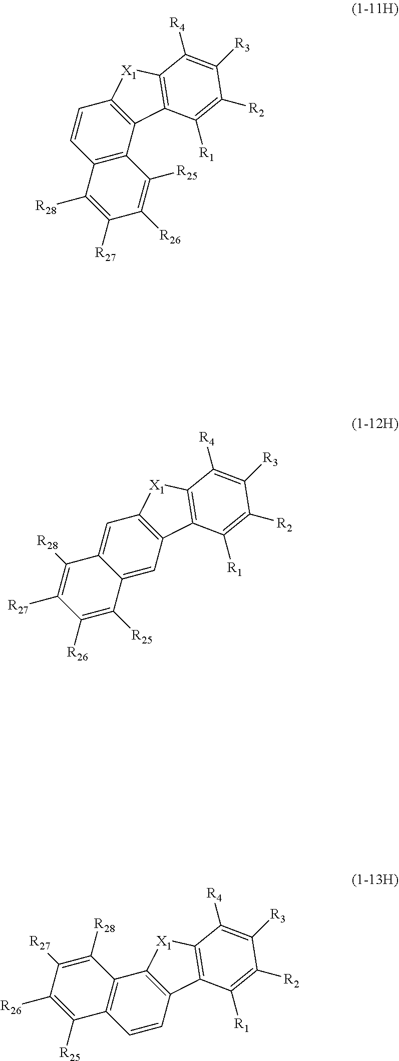
wherein in the formulas (1-11H) to (1-13H), X₁, R₁ to R₄ and R₂₅ to R₂₈ are as defined in the formula (1).

9. The compound according to claim 8, wherein the compound represented by the formula (1) is a compound represented by the formula (1-12H).

10. The compound according to claim 1, wherein the compound represented by the formula (1) is selected from the group consisting of compounds represented by the following formulas (1-24) to (1-26): 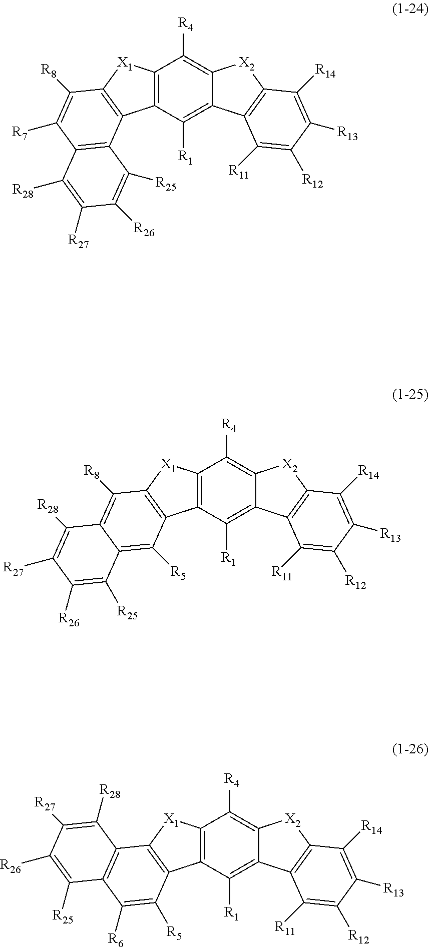
wherein in the formulas (1-24) to (1-26), X₁, X₂, R₁, R₄ to R₅, R₁₁ to R₁₄ and R₂₅ to R₂₈ are as defined in the formula (1).

11. The compound according to claim 10, wherein the compound represented by the formula (1) is a compound represented by the formula (1-25).

12. The compound according to claim 1, wherein the compound represented by the formula (1) is selected from the group consisting of compounds represented by the following formulas (1-31) to (1-35): 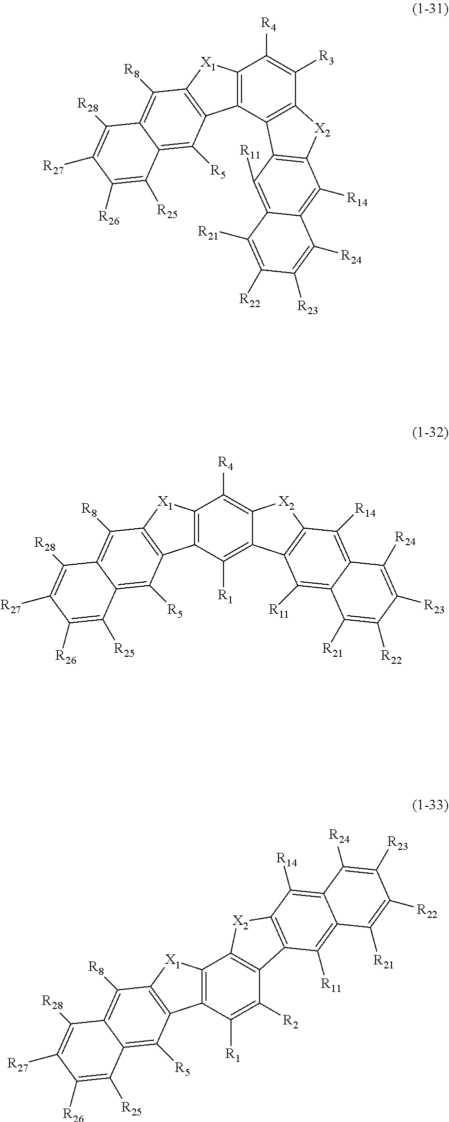 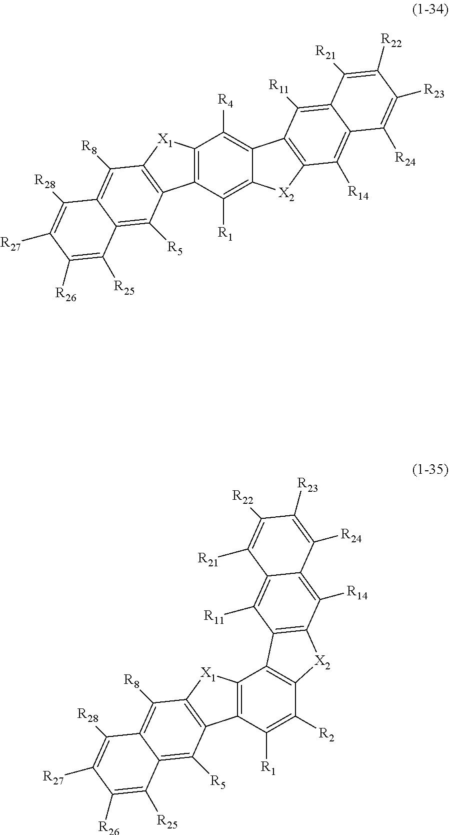
wherein in the formulas (1-31) to (1-35), X₁, X₂, R₁ to R₅, R₈, R₁₁, R₁₄ and R₂₁ to R₂₈ are as defined in the formula (1).

13. The compound according to claim 12, wherein the compound represented by the formula (1) is a compound represented by the formula (1-32).

14. The compound according to claim 1, wherein one of R₂₁ to R₂₄ and one of R₂₅ to R₂₈ are independently a group represented by the formula (13).

15. The compound according to claim 12, wherein the compound represented by the formula (1-32) is a compound represented by the following formula (1-40): 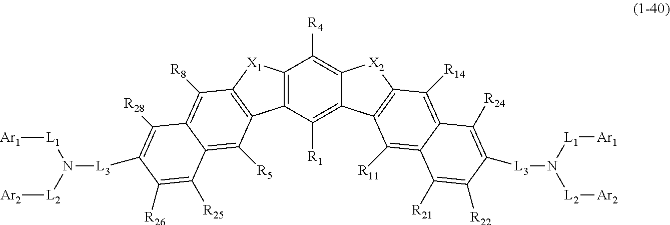
wherein in the formula (1-40), X₁, X₂, R₁, R₄, R₅, R₈, R₁₁, R₁₄, R₂₁, R₂₂, R₂₄ to R₂₆, R₂₈, L₁ to L₃, Ar₁ and Ar₂ are as defined in the formula (1); and
L₁ to L₃, Ar₁ and Ar₂ present in plural may be the same or different.

16. The compound according to claim 1, wherein X₁ and X₂ are O.

17. (canceled)

18. The compound according to claim 1, wherein among R₂₁ to R₂₈, R₁ to R₄ which are not bonded with a divalent group represented by the formula (11), R₁₁ to R₁₄ which are not bonded with a divalent group represented by the formula (12a), and R₅ to R₈ which are not bonded with a divalent group represented by the formula (12b), moieties other than the group represented by the formula (13) are hydrogen atoms.

19. The compound according to claim 1, wherein L₃ is a single bond.

20. The compound according to claim 1, wherein Ar₁ and Ar₂ are independently a substituted or unsubstituted aryl group including 6 to 50 ring carbon atoms.

21. The compound according to claim 1, wherein one of Ar₁ and Ar₂ is a substituted or unsubstituted aryl group including 6 to 50 ring carbon atoms and the other of Ar₁ and Ar₂ is a substituted or unsubstituted monovalent heterocyclic group including 5 to 50 ring atoms.

22. (canceled)

23. The compound according to claim 1, wherein the substituent in the case of “substituted or unsubstituted” and the substituent represented by R are selected from the group consisting of an alkyl group including 1 to 50 carbon atoms, a haloalkyl group including 1 to 50 carbon atoms, an alkenyl group including 2 to 50 carbon atoms, an alkynyl group including 2 to 50 carbon atoms, a cycloalkyl group including 3 to 50 ring carbon atoms, an alkoxy group including 1 to 50 carbon atoms, an alkylthio group including 1 to 50 carbon atoms, an aryloxy group including 6 to 50 ring carbon atoms, an arylthio group including 6 to 50 ring carbon atoms, an aralkyl group including 7 to 50 carbon atoms, —Si(R₄₁)(R₄₂)(R₄₃), —C(═O)R₄₄, —COOR₄₅, —S(═O)₂R₄₆, —P(═O)(R₄₇)(R₄₈), —Ge(R₄₉)(R₅₀)(R₅₁), —N(R₅₂)(R₅₃), a hydroxy group, a halogen atom, a cyano group, a nitro group, an aryl group including 6 to 50 ring carbon atoms, and a monovalent heterocyclic group including 5 to 50 ring atoms,
provided that R₄₁ to R₅₃ are independently a hydrogen atom, an alkyl group including 1 to 50 carbon atoms, an aryl group including 6 to 50 ring carbon atoms, or a monovalent heterocyclic group including 5 to 50 ring atoms, and provided that, when two or more of each of R₄₁ to R₅₃ are present, the two or more of each of R₄₁ to R₅₃ may be the same or different.

24. The compound according to claim 23, wherein the substituent in the case of “substituted or unsubstituted” and the substituent represented by R are selected from the group consisting of an alkyl group including 1 to 50 carbon atoms, an aryl group including 6 to 50 ring carbon atoms and a monovalent heterocyclic group including 5 to 50 ring atoms.

25. The compound according to claim 23, wherein the substituent in the case of “substituted or unsubstituted” and the substituent represented by R are selected from the group consisting of an alkyl group including 1 to 18 carbon atoms, an aryl group including 6 to 18 ring carbon atoms and a monovalent heterocyclic group including 5 to 18 ring atoms.

26. The compound according to claim 1, wherein the compound is a material for an organic electroluminescence device.

27. A material for an organic electroluminescence device, comprising the compound according to claim 1.

28. An organic electroluminescence device, comprising:
a cathode;
an anode; and
at least one organic layer disposed between the cathode and the anode,
wherein at least one layer of the at least one organic layer comprises the compound according to claim 1.

29. The organic electroluminescence device according to claim 28, wherein the at least one organic layer comprises an emitting layer, and the emitting layer comprises the compound.

30. The organic electroluminescence device according to claim 29, wherein the emitting layer further comprises a compound represented by the following formula (2): 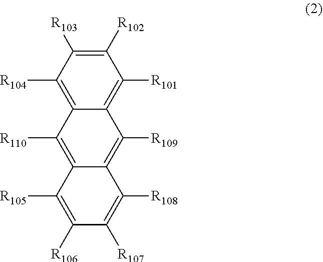
wherein in the formula (2),
one or more pairs of adjacent two or more among R₁₀₁ to R₁₁₀ may form a substituted or unsubstituted, saturated or unsaturated ring;
R₁₀₁ to R₁₁₀ which do not form the substituted or unsubstituted, saturated or unsaturated ring are independently a hydrogen atom, a substituted or unsubstituted alkyl group including 1 to 50 carbon atoms, a substituted or unsubstituted alkenyl group including 2 to 50 carbon atoms, a substituted or unsubstituted alkynyl group including 2 to 50 carbon atoms, a substituted or unsubstituted cycloalkyl group including 3 to 50 ring carbon atoms, a substituted or unsubstituted alkoxy group including 1 to 50 carbon atoms, a substituted or unsubstituted alkylthio group including 1 to 50 carbon atoms, a substituted or unsubstituted aryloxy group including 6 to 50 ring carbon atoms, a substituted or unsubstituted arylthio group including 6 to 50 ring carbon atoms, a substituted or unsubstituted aralkyl group including 7 to 50 carbon atoms, —Si(R₁₂₁)(R₁₂₂)(R₁₂₃), —C(═O)R₂₄, —COOR₁₂₅, —N(R₁₂₆)(R₁₂₇), a halogen atom, a cyano group, a nitro group, a substituted or unsubstituted aryl group including 6 to 50 ring carbon atoms, a substituted or unsubstituted monovalent heterocyclic group including 5 to 50 ring atoms, or a group represented by the following formula (21);
R₁₂₁ to R₁₂₇ are independently a hydrogen atom, a substituted or unsubstituted alkyl group including 1 to 50 carbon atoms, a substituted or unsubstituted aryl group including 6 to 50 ring carbon atoms, or a substituted or unsubstituted monovalent heterocyclic group including 5 to 50 ring atoms, and when two or more R₁₂₁ to R₁₂₇ are present, the two or more R₁₂₁ to R₁₂₇ may be the same or different;
provided that at least one of R₁₀₁ to R₁₁₀ which do not form the substituted or unsubstituted, saturated or unsaturated ring is a group represented by the following formula (21), and when two or more groups represented by the following formula (21) are present, the two or more groups represented by the following formula (21) may be the same or different:
-L₁₀₁-Ar₁₀₁ (21)
wherein in the formula (21),
L₁₀₁ is a single bond, a substituted or unsubstituted arylene group including 6 to 30 ring carbon atoms, or a substituted or unsubstituted divalent heterocyclic group including 5 to 30 ring atoms; and
Ar₁₀₁ is a substituted or unsubstituted aryl group including 6 to 50 ring carbon atoms, or a substituted or unsubstituted monovalent heterocyclic group including 5 to 50 ring atoms.

31. The organic electroluminescence device according to claim 30, wherein at least one of R₁₀₉ and R₁₁₀ is a group represented by the formula (21).

32. The organic electroluminescence device according to claim 3, wherein R₁₀₉ and R₁₁₀ are independently a group represented by the formula (21).

33. The organic electroluminescence device according to claim 30, wherein the compound represented by the formula (2) is a compound represented by the following formula (2-1) or (2-2): 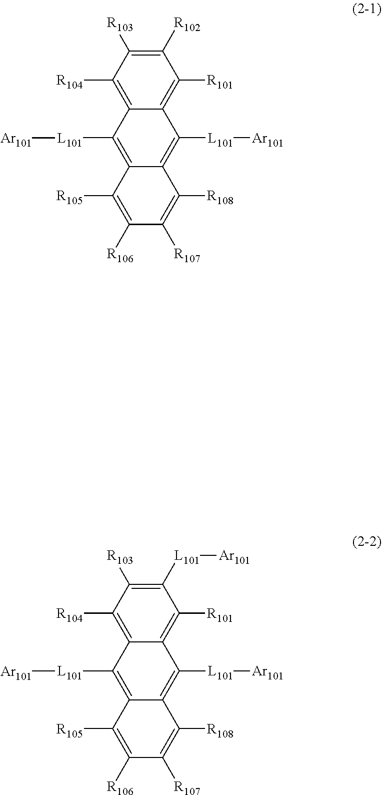
wherein in the formula (2-1), R₁₀₁ to R₁₀₈, L₁₀₁ and Ar₁₀₁ are as defined in the formula (2), and in the formula (2-2), R₁₀₁, R₁₀₃ to R₁₀₈, L₁₀₁ and Ar₁₀₁ are as defined in the formula (2).

34. The organic electroluminescence device according to claim 30, wherein the compound represented by the formula (2) is a compound represented by the following formula (2-3): 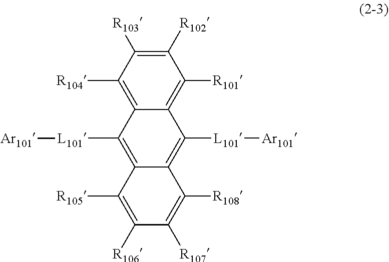
wherein in the formula (2-3),
R_{101′} to R_{108′} are independently a hydrogen atom, or a substituted or unsubstituted aryl group including 6 to 50 ring carbon atoms;
L_{101′} is a substituted or unsubstituted arylene group including 6 to 50 ring carbon atoms, and two L_{101′} s may be the same or different; and
Ar_{101′} is a substituted or unsubstituted aryl group including 6 to 50 ring carbon atoms, and two Ar_{101′}s may be the same or different.

35. The organic electroluminescence device according to claim 30, wherein the compound represented by the formula (2) is a compound represented by the following formula (2-4): 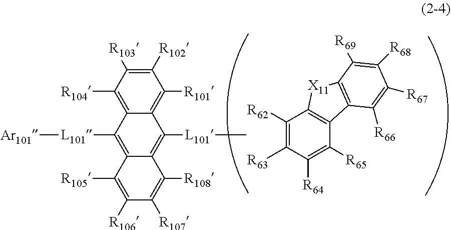
wherein in the formula (2-4),
R_{101′} to R_{108′} are independently a hydrogen atom, or a substituted or unsubstituted aryl group including 6 to 50 ring carbon atoms;
L_{101′} is a substituted or unsubstituted arylene group including 6 to 50 ring carbon atoms;
L_{101″} is a substituted or unsubstituted arylene group including 6 to 50 ring carbon atoms, or a substituted or unsubstituted divalent heterocyclic group including 5 to 50 ring atoms;
Ar_{101″} is a substituted or unsubstituted aryl group including 6 to 50 ring carbon atoms, or a substituted or unsubstituted monovalent heterocyclic group including 5 to 50 ring atoms;
X₁₁ is O, S, or N(R₆₁);
R₆₁ is a hydrogen atom, a substituted or unsubstituted alkyl group including 1 to 50 carbon atoms, or a substituted or unsubstituted aryl group including 6 to 50 ring carbon atoms;
one of R₆₂ to R₆₉ is bonded with L_{101′};
R₆₂ to R₆₉ which are not bonded with L_{101′} are independently a hydrogen atom, a substituted or unsubstituted alkyl group including 1 to 50 carbon atoms, or a substituted or unsubstituted aryl group including 6 to 50 ring carbon atoms; and
one or more pairs of adjacent two or more among R₆₂ to R₆₉ which are not bonded with L_{101′} may be bonded with each other to form a substituted or unsubstituted, saturated or unsaturated ring.

36. The organic electroluminescence device according to claim 30, wherein the compound represented by the formula (2) is a compound represented by the following formula (2-6): 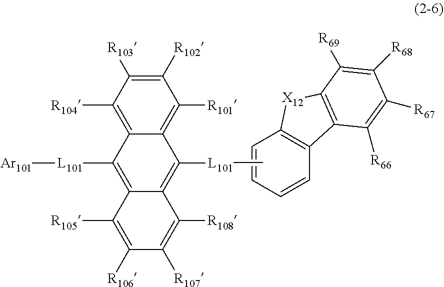
wherein in the formula (2-6), L₁₀₁ and Ar₁₀₁ are as defined in the formula (2);
R₁₀₁ to R_{108′} are as defined in the formula (2-4);
R₆₆ to R₆₉ are independently a hydrogen atom, a substituted or unsubstituted alkyl group including 1 to 50 carbon atoms, or a substituted or unsubstituted aryl group including 6 to 50 ring carbon atoms, provided that R₆₆ and R₆₇, R₆₇ and R₆₈, and R₆₉ and R₆₇ are not bonded with each other to form a ring; and
X₁₂ is O or S.

37. The organic electroluminescence device according to claim 30, wherein the compound represented by the formula (2) is a compound represented by the following formula (2-7): 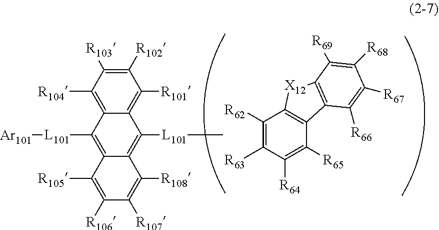
wherein in the formula (2-7), L₁₀₁ and Ar₁₀₁ are as defined in the formula (2);
R₁₀₁ to R_{108′} are independently a hydrogen atom, or a substituted or unsubstituted aryl group including 6 to 50 ring carbon atoms;
one of R₆₂ to R₆₉ is bonded with L₁₀₁;
R₆₂ to R₆₉ which do not bonded with L₁₀₁ are independently a hydrogen atom, a substituted or unsubstituted alkyl group including 1 to 50 carbon atoms, or a substituted or unsubstituted aryl group including 6 to 50 ring carbon atoms, provided that one pair among R₆₆ and R₆₇, R₆₇ and R₆₈, and R₆₈ and R₆₉ is bonded with each other to form a substituted or unsubstituted, saturated or unsaturated ring.

38. The organic electroluminescence device according to claim 30, wherein the compound represented by the formula (2) is a compound represented by the following formula (2-8): 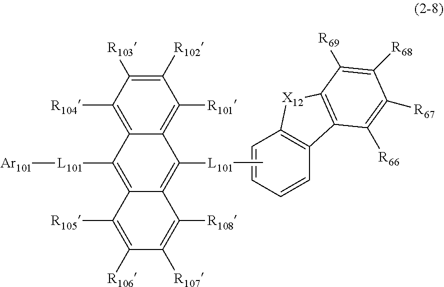
wherein in the formula (2-8), L₁₀₁ and Ar₁₀₁ are as defined in the formula (2);
R₁₀₁ to R_{108′} are as defined in the formula (2-7);
R₆₆ to R₆₉ are independently a hydrogen atom, a substituted or unsubstituted alkyl group including 1 to 50 carbon atoms, or a substituted or unsubstituted aryl group including 6 to 50 ring carbon atoms, provided that one pair among R₆₆ and R₆₇, R₆₇ and R₆₈, and R₆₈ and R₆₉ is bonded with each other to form a substituted or unsubstituted, saturated or unsaturated ring.

39. The organic electroluminescence device according to claim 37, wherein one pair among R₆₆ and R₆₇, R₆₇ and R₆₈, and R₆₈ and R₆₉ is bonded with each other to form a ring represented by the following formula (2-8-1) or (2-8-2);
R₆₆ to R₆₉ which do not form a ring represented by the formula (2-8-1) or (2-8-2) do not form a substituted or unsubstituted, saturated or unsaturated ring: 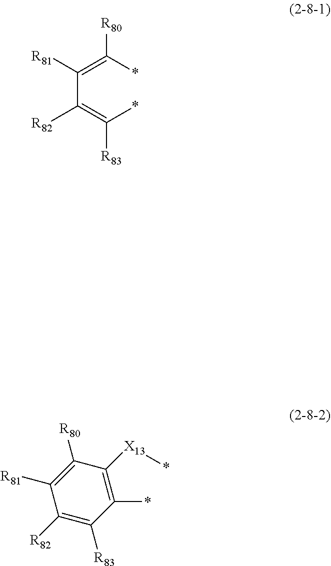
wherein in the formulas (2-8-1) and (2-8-2),
two “*” are independently bonded with one pair of R₆₆ and R₆₇, R₆₇ and R₆₈, and R₆₈ and R₆₉;
R₈₀ to R₈₃ are independently a hydrogen atom, a substituted or unsubstituted alkyl group including 1 to 50 carbon atoms, or a substituted or unsubstituted aryl group including 6 to 50 ring carbon atoms; and
X₁₃ is O or S.

40. The organic electroluminescence device according to claim 30, wherein the compound represented by the formula (2) is represented by the following formula (2-9) 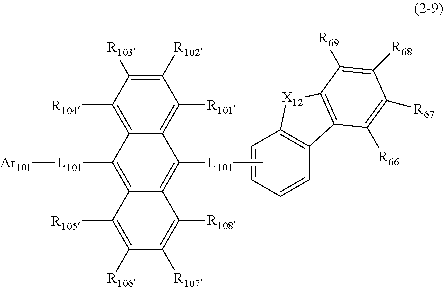
wherein in the formula (2-9),
L₁₀₁ and Ar₁₀₁ are as defined in the formula (2);
R_{101′} to R_{108′} are as defined in the formula (2-4);
R₆₆ to R₆₉ are independently a hydrogen atom, a substituted or unsubstituted alkyl group including 1 to 50 carbon atoms, or a substituted or unsubstituted aryl group including 6 to 50 ring carbon atoms, provided that R₆₆ and R₆₇, R₆₇ and R₆₈, and R₆₉ and R₆₇ are not bonded with each other and do not form a substituted or unsubstituted, saturated or unsaturated ring; and
X₁₂ is O or S.

41. The organic electroluminescence device according to claim 30, wherein the compound represented by the formula (2) is represented by the following formula (2-4A): 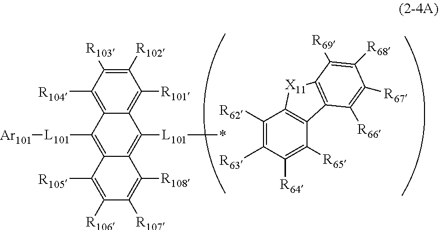
wherein in the formula (2-4A),
L₁₀₁ and Ar₁₀₁ are as defined in the formula (2);
R₁₀₁ to R_{108′} are independently a hydrogen atom, or a substituted or unsubstituted aryl group including 6 to 50 ring carbon atoms;
X₁₁ is O, S, or N(R₆₁);
R₆₁ is a hydrogen atom, a substituted or unsubstituted alkyl group including 1 to 50 carbon atoms, or a substituted or unsubstituted aryl group including 6 to 50 ring carbon atoms;
one or more pairs of adjacent two or more among R_{62′} to R_{69′} may form a substituted or unsubstituted, saturated or unsaturated ring, and adjacent two among R_{62′} to R_{69′} form a ring represented by the following formula (2-4A-1);
R_{62′} to R_{69′} which do not form a substituted or unsubstituted, saturated or unsaturated ring are independently a hydrogen atom, a substituted or unsubstituted alkyl group including 1 to 50 carbon atoms, or a substituted or unsubstituted aryl group including 6 to 50 ring carbon atoms; 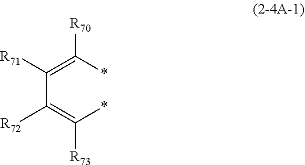
wherein in the formula (2-4A-1),
two “*” are independently bonded with adjacent two among R_{62′} to R_{69′};
one of R₇₀ to R₇₃ is bonded with L₁₀₁; and
R₇₀ to R₇₃ which do not bonded with L₁₀₁ are independently a hydrogen atom, a substituted or unsubstituted alkyl group including 1 to 50 carbon atoms, or a substituted or unsubstituted aryl group including 6 to 50 ring carbon atoms.

42. The organic electroluminescence device according to claim 29, which further comprises a hole-transporting layer between the anode and the emitting layer.

43. The organic electroluminescence device according to claim 29, which further comprises an electron-transporting layer between the cathode and the emitting layer.

44. An electronic appliance wherein the organic electroluminescence device according to claim 28 is provided.
